(19) United States
(12) Patent Application Publication
(10) Pub. No.: US 2023/0354706 A1
(43) Pub. Date: Nov. 2, 2023

(54) ORGANIC LIGHT EMITTING DEVICE

(71) Applicant: LG CHEM, LTD., Seoul (KR)

(72) Inventors: Minjun KIM, Daejeon (KR); Dong Hoon LEE, Daejeon (KR); Sang Duk SUH, Daejeon (KR); Young Seok KIM, Daejeon (KR); Donghee KIM, Daejeon (KR); Joongsuk OH, Daejeon (KR); Da Jung LEE, Daejeon (KR)

(21) Appl. No.: 18/016,720

(22) PCT Filed: Oct. 22, 2021
(86) PCT No.: PCT/KR2021/014973

§ 371 Date: Jan. 18, 2023

(30) Foreign Application Priority Data
Oct. 22, 2020 (KR) 10-2020-0137751
Oct. 22, 2021 (KR) 10-2021-0142117

(51) Int. Cl. H10K 85/60; C07D 405/04; C09K 11/06; C07D 409/14; C07D 405/14; C07D 405/10; C07D 209/80; C07D 409/10; C07D 409/12; C07D 405/12

(52) U.S. Cl. CPC H10K 85/6574; C07D 405/04; C09K 11/06; H10K 85/654; H10K 85/615; H10K 85/622; C07D 409/14; H10K 85/6576; C07D 405/14; H10K 85/626; C07D 405/10; H10K 85/6572; C07D 209/80; H10K 85/636; C07D 409/10; H10K 85/633; C07D 409/12; C07D 405/12; H10K 50/11

(57) ABSTRACT

Provided is an organic light emitting device comprising an anode; a cathode; and a light emitting layer between the anode and the cathode, wherein the light emitting layer comprises a compound of the following Chemical Formula 1 and a compound of the following Chemical Formula 2:

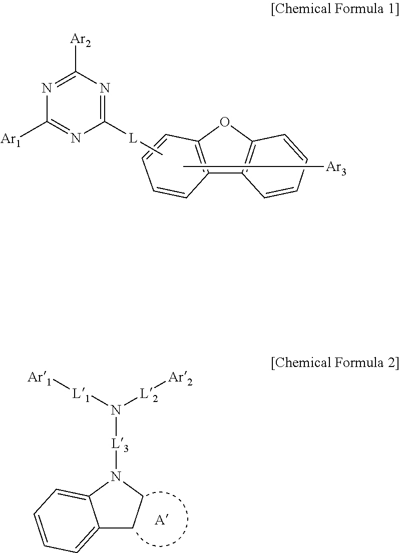

where A′ is a naphthalene ring fused with an adjacent ring and is unsubstituted or substituted with deuterium, and the other substituents are described in the specification. Organic light emitting devices in which a compound of Chemical Formula 1 and a compound of Chemical Formula 2 are co-deposited as a host for a light emitting layer exhibit reduced driving voltage and increased efficiency and lifespan.

### CROSS-REFERENCE TO RELATED APPLICATION(S)

This application is a National Stage Application of International Application No. PCT/KR2021/014973 filed on Oct. 22, 2021, which claims the benefit of Korean Patent Applications No. 10-2020-0137751 filed on Oct. 22, 2020 and No. 10-2021-0142117 filed on Oct. 22, 2021 in the Korean Intellectual Property Office, the disclosures of which are incorporated herein by reference in their entirety.

### TECHNICAL FIELD

The present disclosure relates to an organic light emitting device.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuing need for the development of new materials for the organic materials used in the organic light emitting devices as described above.

### PRIOR ART LITERATURE

(Patent Literature 0001) Korean Unexamined Patent Publication No. 10-2000-0051826

### BRIEF DESCRIPTION

### Technical Problem

The present disclosure relates to an organic light emitting device having improved driving voltage, efficiency, and lifespan.

### Technical Solution

In the present disclosure, provided is an organic light emitting device including
an anode; a cathode; and a light emitting layer between the anode and the cathode,
wherein the light emitting layer comprises a compound of the following Chemical Formula 1 and a compound of the following Chemical Formula 2:

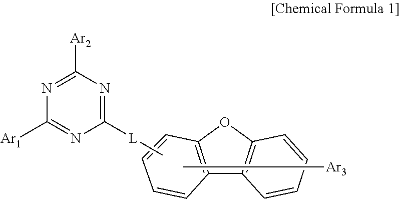

wherein in the Chemical Formula 1:
L is a single bond or substituted or unsubstituted C₆₋₆₀ arylene;
Ar₁ and Ar₂ are each independently a substituted or unsubstituted C₆₋₆₀ aryl, or substituted or unsubstituted C₂₋₆₀ heteroaryl containing at least one heteroatom selected from the group consisting of N, O and S; and
Ar₃ is hydrogen, deuterium, substituted or unsubstituted C₆₋₆₀ aryl, or substituted or unsubstituted C₂₋₆₀ heteroaryl containing at least one heteroatom selected from the group consisting of N, O and S;

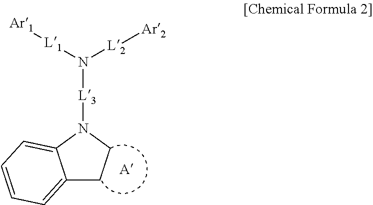

wherein in the Chemical Formula 2:
A′ is a naphthalene ring fused with an adjacent ring and is unsubstituted or substituted with deuterium;
L′₁ and L′₂ are each independently a single bond, substituted or unsubstituted C₆₋₆₀ arylene, or substituted or unsubstituted C₂₋₆₀ heteroarylene containing at least one heteroatom selected from the group consisting of N, O and S;
L′₃ is a substituted or unsubstituted C₆₋₆₀ arylene, or substituted or unsubstituted C₂₋₆₀ heteroarylene containing at least one heteroatom selected from the group consisting of N, O and S; and
Ar′₁ and Ar′₂ are each independently a substituted or unsubstituted C₆₋₆₀ aryl, or substituted or unsubstituted C₂₋₆₀ heteroaryl containing at least one heteroatom selected from the group consisting of N, O and S.

### Advantageous Effects

The above-described organic light emitting device has improved driving voltage, efficiency, and lifespan.

### BRIEF DESCRIPTION OF THE DRAWINGS

FIG. 1 shows an example of an organic light emitting device including a substrate 1, an anode 2, a light emitting layer 3, and a cathode 4.

FIG. 2 shows an example of an organic light emitting device including a substrate 1, an anode 2, a hole injection layer 5, a hole transport layer 6, a light emitting layer 3, an electron transport layer 7, an electron injection layer 8, and a cathode 4.

FIG. 3 shows an example of an organic light emitting device including a substrate 1, an anode 2, a hole injection layer 5, a hole transport layer 6, an electron blocking layer 9, a light emitting layer 3, a hole blocking layer 10, an electron injection and transport layer 11, and a cathode 4.

### DETAILED DESCRIPTION

Hereinafter, embodiments of the present disclosure will be described in more detail to facilitate understanding of the invention.

As used herein, the notation

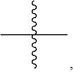

or [symbol] means a bond linked to another substituent group.

As used herein, the term “substituted or unsubstituted” means being unsubstituted or substituted with one or more substituents selected from the group consisting of deuterium, a halogen group, a nitrile group, a nitro group, a hydroxyl group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, and a heterocyclic group containing at least one of N, O and S atoms, or being unsubstituted or substituted with a substituent in which two or more substituents of the above-exemplified substituents are connected. For example, “a substituent in which two or more substituents are connected” can be a biphenyl group. Namely, a biphenyl group can be an aryl group, or it can also be interpreted as a substituent in which two phenyl groups are connected.

In the present disclosure, the carbon number of a carbonyl group is not particularly limited, but is preferably 1 to 40. Specifically, the carbonyl group can be a compound having the following structural formulae, but is not limited thereto:

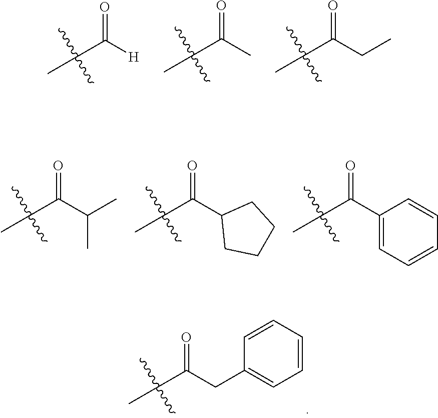

In the present disclosure, an ester group can have a structure in which oxygen of the ester group is substituted by a straight-chain, branched-chain, or cyclic alkyl group having 1 to 25 carbon atoms, or an aryl group having 6 to 25 carbon atoms. Specifically, the ester group can be a compound having the following structural formulae, but is not limited thereto:

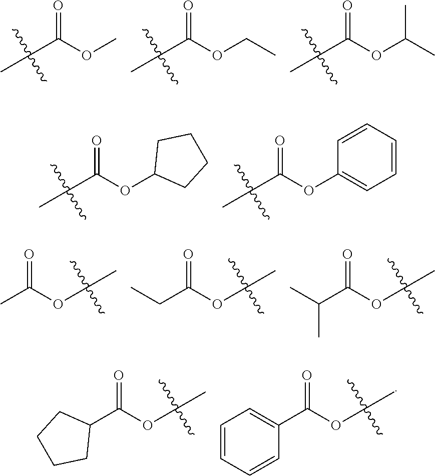

In the present disclosure, the carbon number of an imide group is not particularly limited, but is preferably 1 to 25. Specifically, the imide group can be a compound having the following structural formulae, but is not limited thereto:

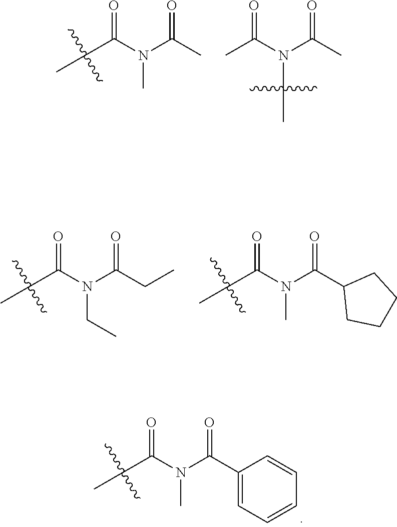

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but is not limited thereto.

In the present disclosure, a boron group specifically includes a trimethylboron group, a triethylboron group, a t-butyldimethylboron group, a triphenylboron group, a phenylboron group and the like, but is not limited thereto.

In the present disclosure, examples of a halogen group include fluorine, chlorine, bromine, or iodine.

In the present disclosure, the alkyl group can be straight-chain, or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)inyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group. According to one embodiment, the carbon number of the aryl group is 6 to 30. According to one embodiment, the carbon number of the aryl group is 6 to 20. The monocyclic aryl group includes a phenyl group, a biphenyl group, a terphenyl group and the like, but is not limited thereto. The polycyclic aryl group includes a naphthyl group, an anthracenyl group, a phenanthryl group, a pyrenyl group, a perylenyl group, a chrysenyl group, a fluorenyl group or the like, but is not limited thereto.

In the present disclosure, a fluorenyl group can be substituted, and two substituents can be bonded to each other to form a spiro structure. In the case where the fluorenyl group is substituted,

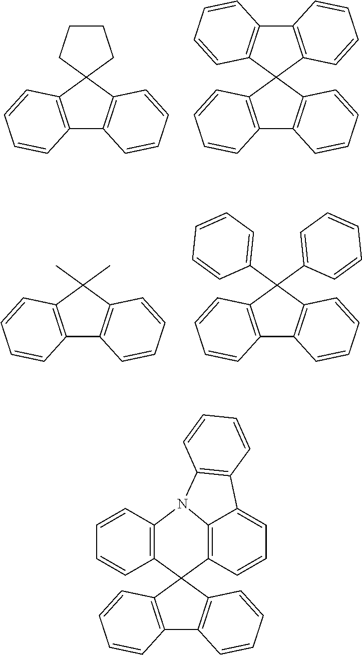

and the like can be formed. However, the structure is not limited thereto.

In the present disclosure, a heterocyclic group is a heterocyclic group containing at least one heteroatom of O, N, Si and S as a heterogeneous element, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group, and the arylamine group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine can apply the aforementioned description of the heterocyclic group. In the present disclosure, the alkenyl group in the aralkenyl group is the same as the aforementioned examples of the alkenyl group. In the present disclosure, the aforementioned description of the aryl group can be applied except that the arylene is a divalent group. In the present disclosure, the aforementioned description of the heterocyclic group can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heterocyclic group can be applied, except that the heterocycle is not a monovalent group but formed by combining two substituent groups.

Hereinafter, the present invention will be described in detail for each configuration.

### Anode and Cathode

The anode and cathode used in the present disclosure refer to electrodes used in an organic light emitting device.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

### Light Emitting Layer

The light emitting layer used in the present disclosure refers to a layer that emits light in the visible light region by combining holes and electrons transported from the anode and the cathode. Generally, the light emitting layer includes a host material and a dopant material. In the present disclosure, the compound of Chemical Formula 1 and the compound of Chemical Formula 2 are included as hosts.

Chemical Formula 1 can be the following Chemical Formula 1-1 depending on the bonding position of dibenzofuran and triazine:

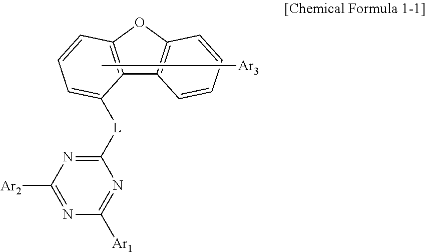

wherein in Chemical Formula 1-1, L and Ar₁ to Ar₂ are as defined in Chemical Formula 1.

In addition, Chemical Formula 1-1 can be the following Chemical Formula 1-1-a depending on the bonding position of Ara:

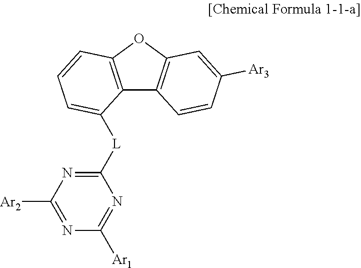

wherein in Chemical Formula 1-1-a, L and Ar₁ to Ar₃ are as defined in Chemical Formula 1.

Preferably, L is a single bond or substituted or unsubstituted C₆₋₂₀ arylene, Ar₁ and Ar₂ are each independently a substituted or unsubstituted C₆₋₂₀ aryl or substituted or unsubstituted C₂₋₂₀ heteroaryl containing at least one heteroatom selected from the group consisting of N, O and S, and Ar₃ is a substituted or unsubstituted C₆₋₂₀ aryl or substituted or unsubstituted C₂₋₂₀ heteroaryl containing at least one heteroatom selected from the group consisting of N, O and S.

Preferably, L is a single bond, phenylene, or naphthalenediyl.

Preferably, Ar₁ and Ar₂ are each independently phenyl, biphenylyl, terphenylyl, naphthyl, phenanthrenyl, phenylnaphthyl, naphthylphenyl, dibenzofuranyl, or dibenzothiophenyl.

Preferably, Ar₃ is phenyl, biphenylyl, terphenylyl, naphthyl, phenanthrenyl, phenylnaphthyl, naphthylphenyl, triphenylenyl, dibenzofuranyl, dibenzothiophenyl, benzonaphthofuranyl, or benzonaphthothiophenyl.

Preferably, Ar₃ is biphenylyl, terphenylyl, naphthyl, phenanthrenyl, phenylnaphthyl, naphthylphenyl, triphenylenyl, benzonaphthofuranyl, benzonaphthothiophenyl, or fluoranthenyl.

Preferably, Ar₁ to Ar₃ can each independently be a substituted or unsubstituted C₆₋₂₀ aryl.

Preferably, Ar₁ and Ar₂ can each independently be terphenylyl, naphthyl, phenanthrenyl, phenylnaphthyl, or naphthylphenyl.

Preferably, Ar₃ is biphenylyl, terphenylyl, naphthyl, phenanthrenyl, phenylnaphthyl, naphthylphenyl, or triphenylenyl.

Preferably, when Chemical Formula 1 is a compound of Chemical Formula 1-1-a, Ar₃ is naphthyl.

Representative examples of the compound of Chemical Formula 1 are as follows:

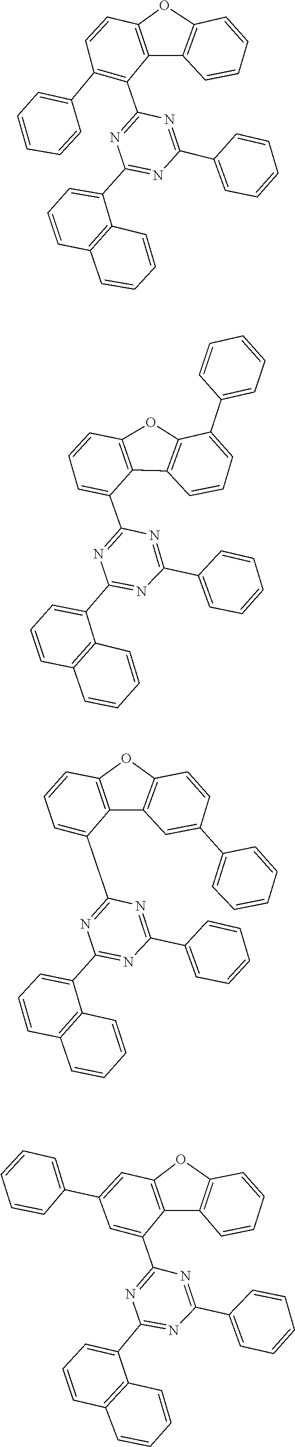 [SMILES: c1ccc(-c2nc(-c3cccc4ccccc34)nc(-c3c(-c4ccccc4)ccc4oc5ccccc5c34)n2)cc1 ; c1ccc(-c2nc(-c3cccc4ccccc34)nc(-c3cccc4oc5c(-c6ccccc6)cccc5c34)n2)cc1 ; c1ccc(-c2ccc3oc4cccc(-c5nc(-c6ccccc6)nc(-c6cccc7ccccc67)n5)c4c3c2)cc1 ; c1ccc(-c2cc(-c3nc(-c4ccccc4)nc(-c4cccc5ccccc45)n3)c3c(c2)oc2ccccc23)cc1]

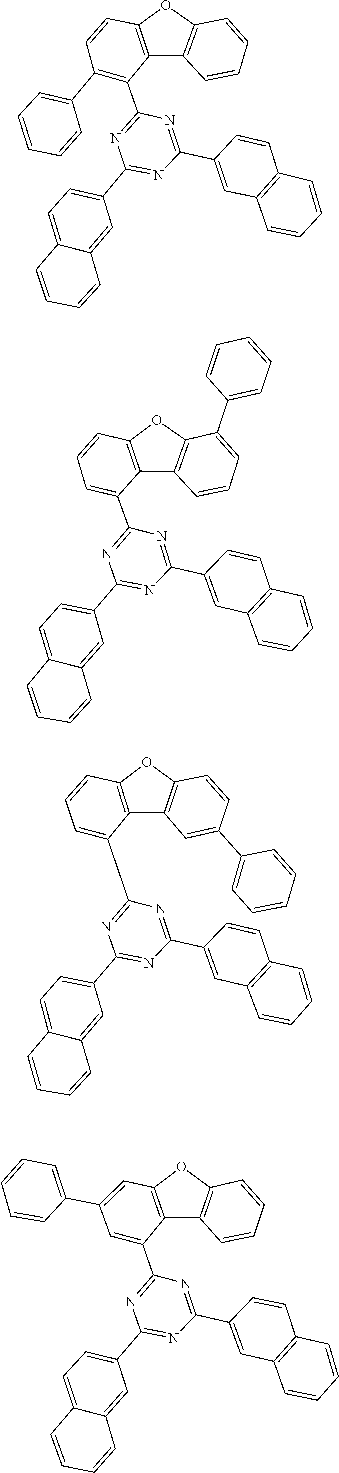 [SMILES: c1ccc(-c2ccc3oc4ccccc4c3c2-c2nc(-c3ccc4ccccc4c3)nc(-c3ccc4ccccc4c3)n2)cc1 ; c1ccc(-c2cccc3c2oc2cccc(-c4nc(-c5ccc6ccccc6c5)nc(-c5ccc6ccccc6c5)n4)c23)cc1 ; c1ccc(-c2ccc3oc4cccc(-c5nc(-c6ccc7ccccc7c6)nc(-c6ccc7ccccc7c6)n5)c4c3c2)cc1 ; c1ccc(-c2cc(-c3nc(-c4ccc5ccccc5c4)nc(-c4ccc5ccccc5c4)n3)c3c(c2)oc2ccccc23)cc1]

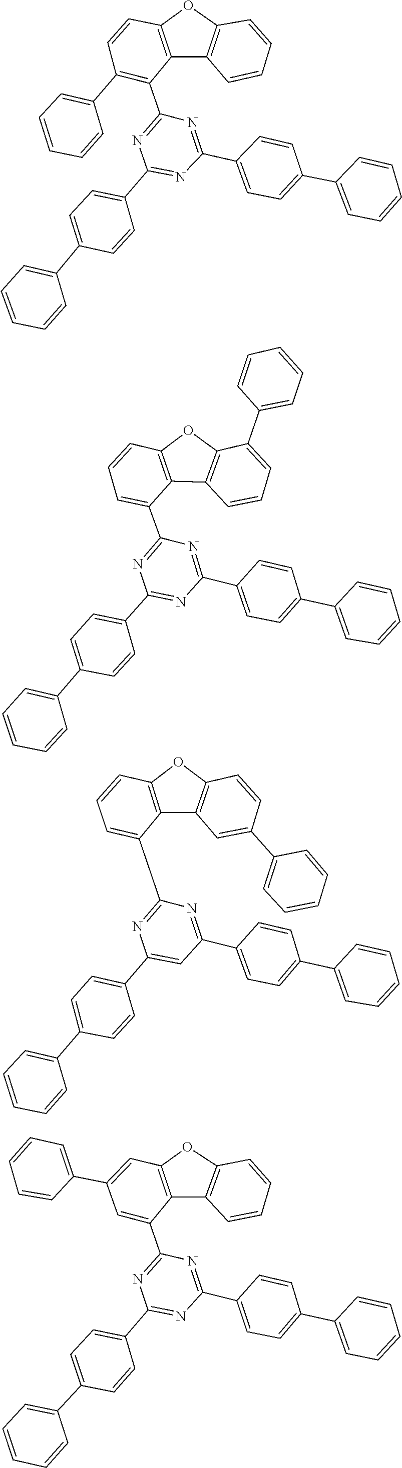

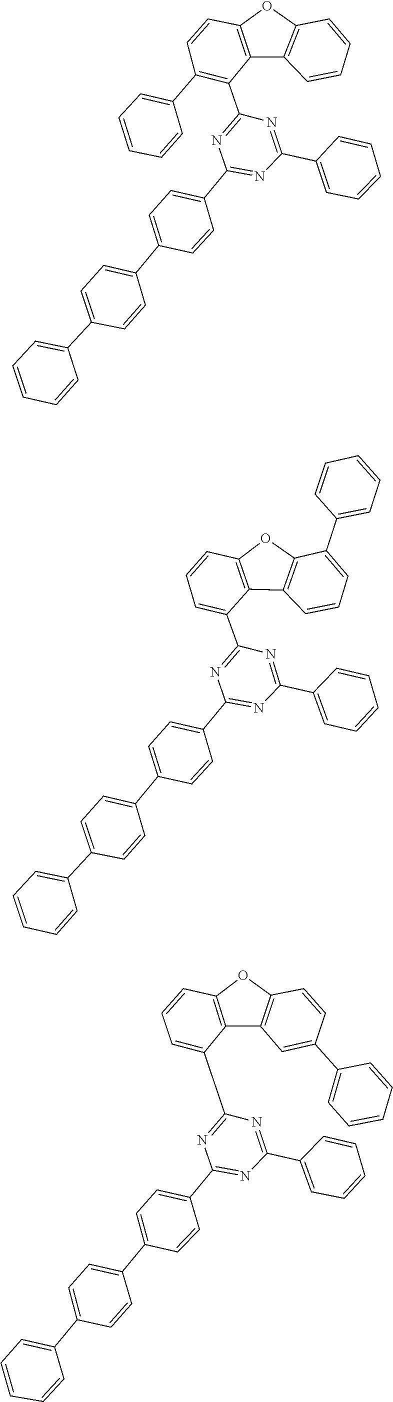 [SMILES: c1ccc(-c2ccc(-c3ccc(-c4nc(-c5ccccc5)nc(-c5c(-c6ccccc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccccc8)cccc7c56)n4)cc3)cc2)cc1 ; c1ccc(-c2ccc(-c3ccc(-c4nc(-c5ccccc5)nc(-c5cccc6oc7ccc(-c8ccccc8)cc7c56)n4)cc3)cc2)cc1]

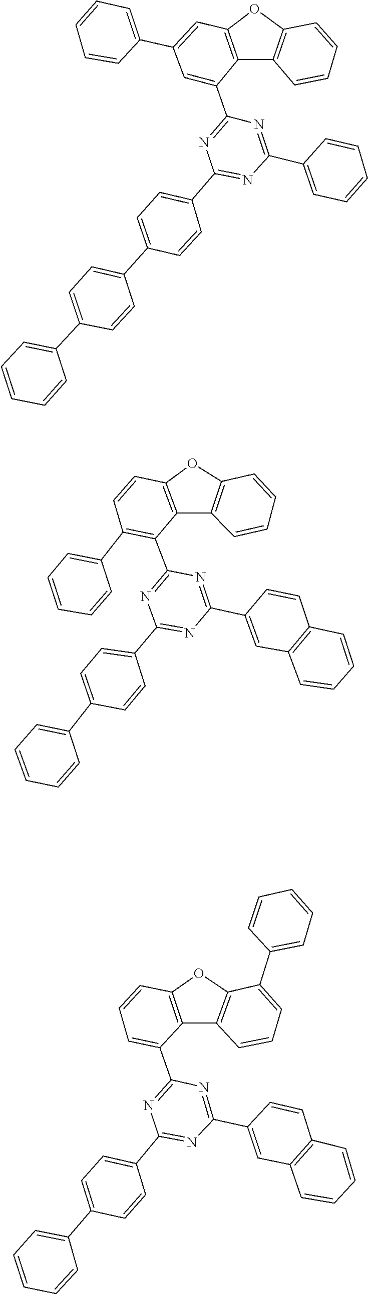 [SMILES: c1ccc(-c2ccc(-c3ccc(-c4nc(-c5ccccc5)nc(-c5cc(-c6ccccc6)cc6oc7ccccc7c56)n4)cc3)cc2)cc1 ; c1ccc(-c2ccc(-c3nc(-c4ccc5ccccc5c4)nc(-c4c(-c5ccccc5)ccc5oc6ccccc6c45)n3)cc2)cc1 ; c1ccc(-c2ccc(-c3nc(-c4ccc5ccccc5c4)nc(-c4cccc5oc6c(-c7ccccc7)cccc6c45)n3)cc2)cc1]

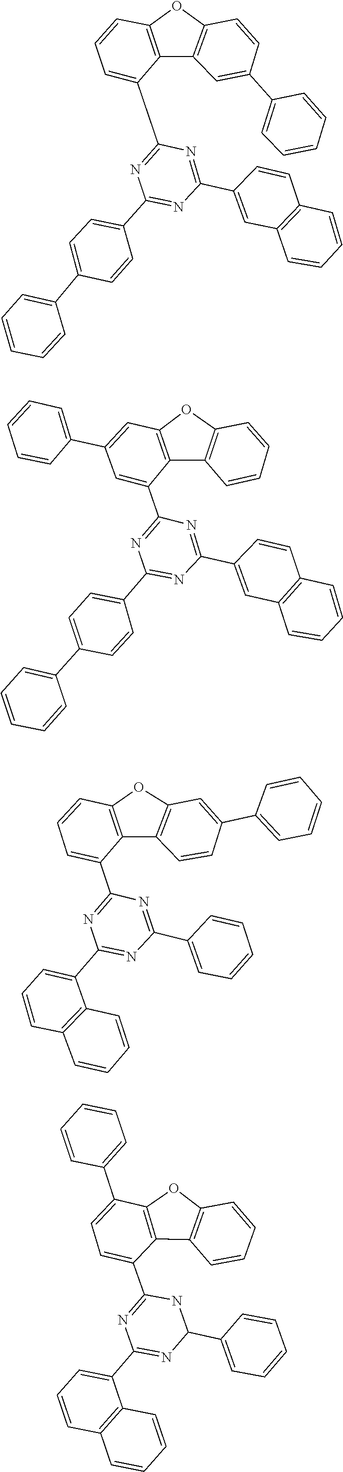

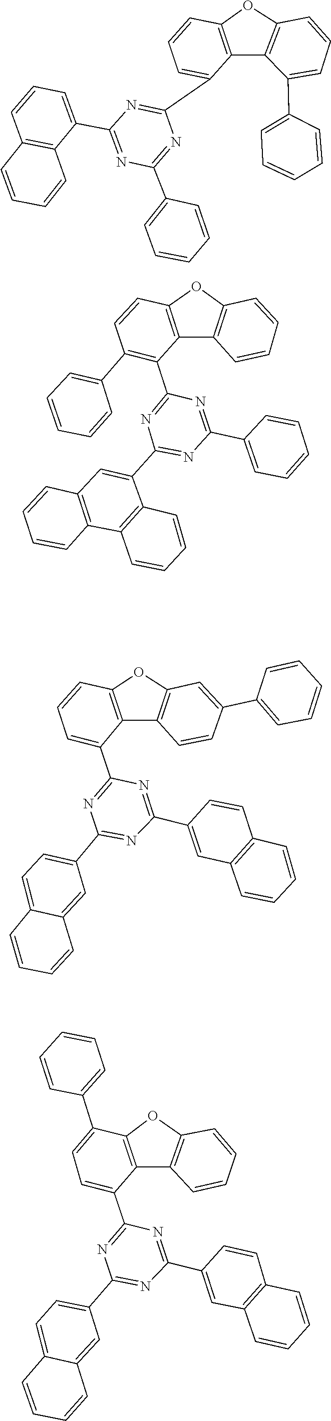

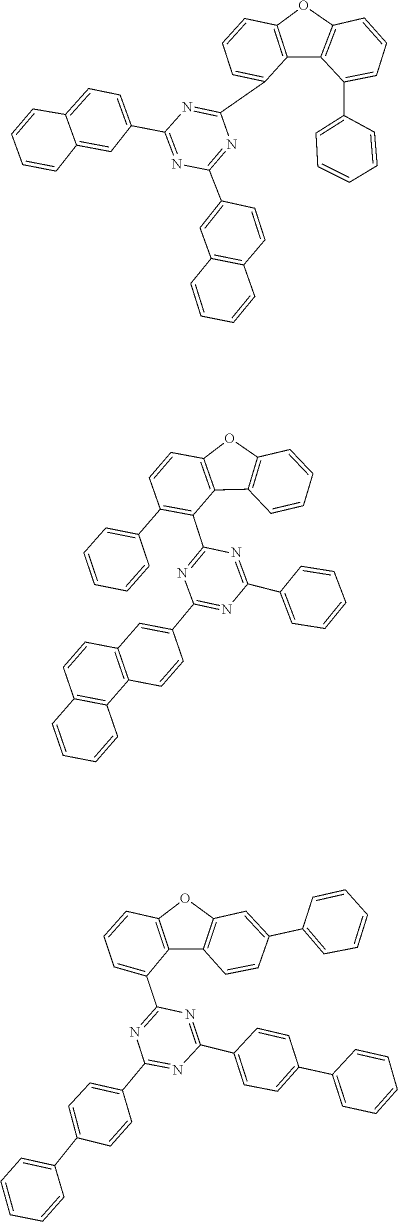 [SMILES: c1ccc(-c2cccc3oc4cccc(-c5nc(-c6ccc7ccccc7c6)nc(-c6ccc7ccccc7c6)n5)c4c23)cc1 ; c1ccc(-c2nc(-c3ccc4c(ccc5ccccc54)c3)nc(-c3c(-c4ccccc4)ccc4oc5ccccc5c34)n2)cc1 ; c1ccc(-c2ccc(-c3nc(-c4ccc(-c5ccccc5)cc4)nc(-c4cccc5oc6cc(-c7ccccc7)ccc6c45)n3)cc2)cc1]

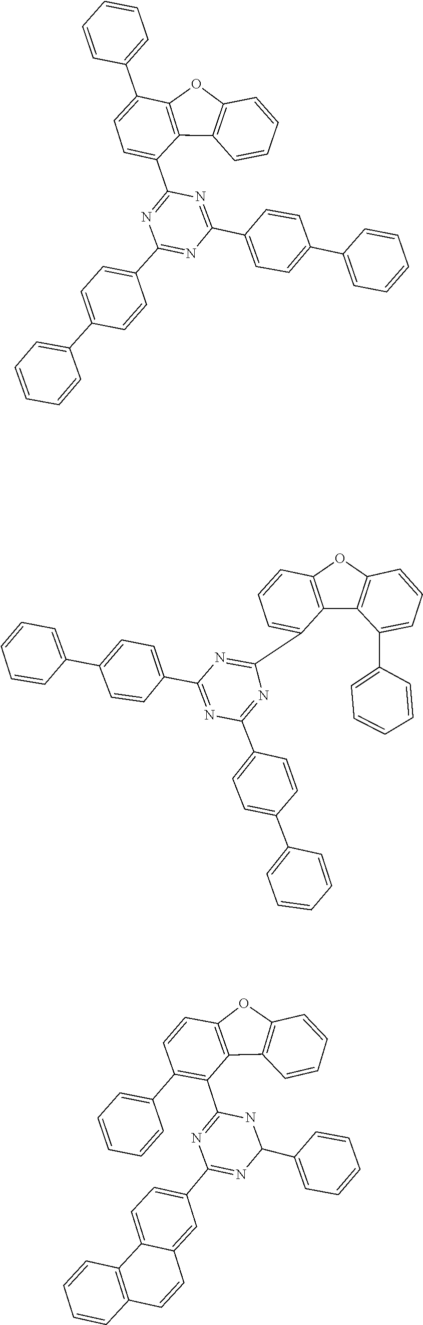

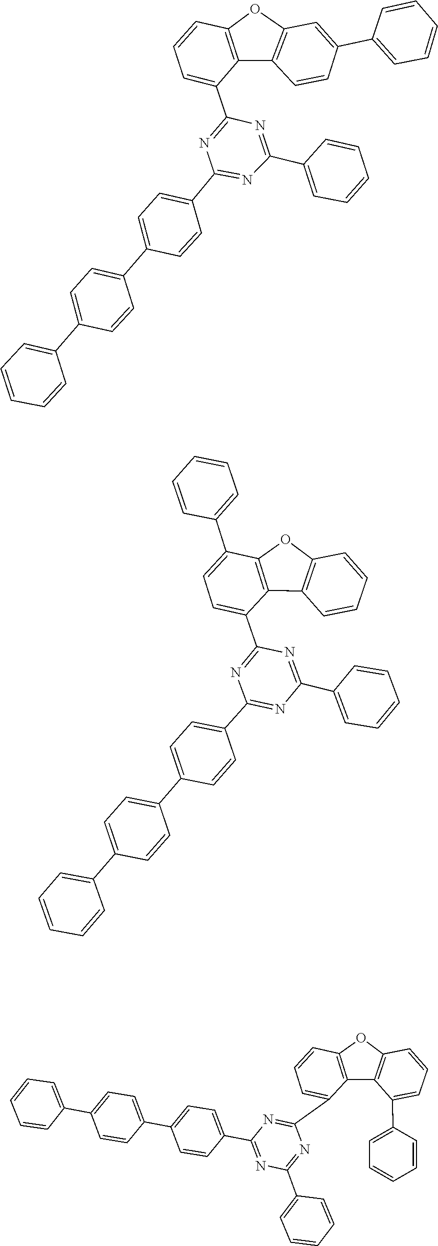 [SMILES: c1ccc(-c2ccc(-c3ccc(-c4nc(-c5ccccc5)nc(-c5cccc6oc7cc(-c8ccccc8)ccc7c56)n4)cc3)cc2)cc1 ; c1ccc(-c2ccc(-c3ccc(-c4nc(-c5ccccc5)nc(-c5ccc(-c6ccccc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccccc8)c7c56)n4)cc3)cc2)cc1]

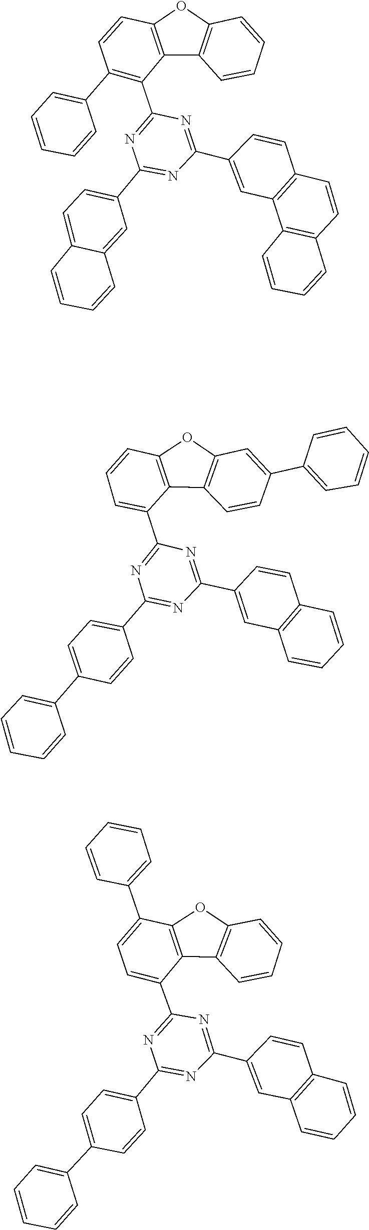 [SMILES: c1ccc(-c2ccc3oc4ccccc4c3c2-c2nc(-c3ccc4ccccc4c3)nc(-c3ccc4ccc5ccccc5c4c3)n2)cc1 ; c1ccc(-c2ccc(-c3nc(-c4ccc5ccccc5c4)nc(-c4cccc5oc6cc(-c7ccccc7)ccc6c45)n3)cc2)cc1 ; c1ccc(-c2ccc(-c3nc(-c4ccc5ccccc5c4)nc(-c4ccc(-c5ccccc5)c5oc6ccccc6c45)n3)cc2)cc1]

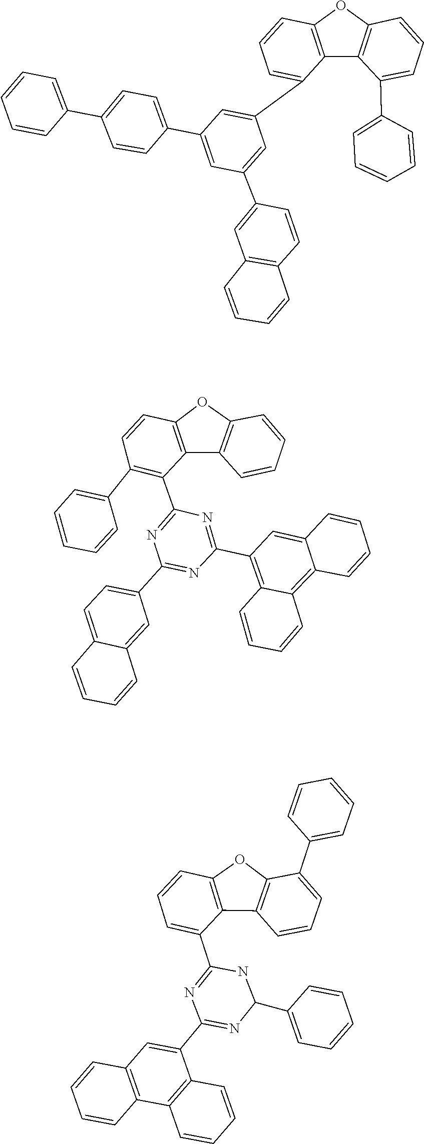

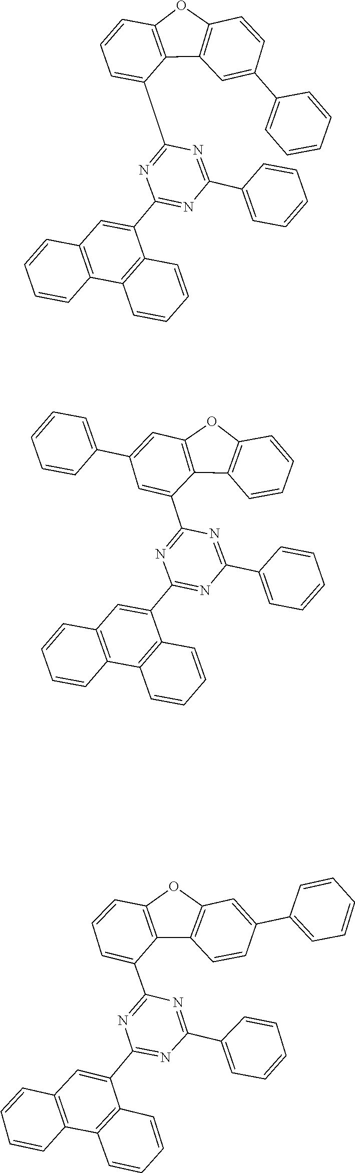 [SMILES: c1ccc(-c2ccc3oc4cccc(-c5nc(-c6ccccc6)nc(-c6cc7ccccc7c7ccccc67)n5)c4c3c2)cc1 ; c1ccc(-c2cc(-c3nc(-c4ccccc4)nc(-c4cc5ccccc5c5ccccc45)n3)c3c(c2)oc2ccccc23)cc1 ; c1ccc(-c2ccc3c(c2)oc2cccc(-c4nc(-c5ccccc5)nc(-c5cc6ccccc6c6ccccc56)n4)c23)cc1]

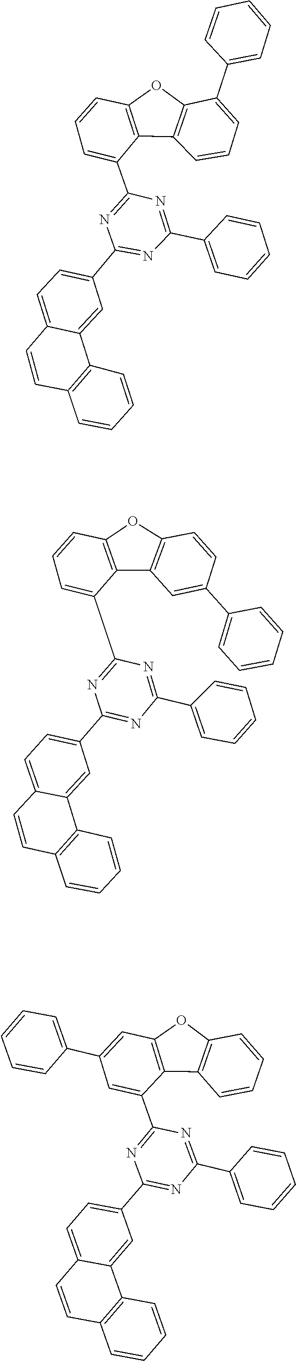 [SMILES: c1ccc(-c2nc(-c3ccc4ccc5ccccc5c4c3)nc(-c3cccc4oc5c(-c6ccccc6)cccc5c34)n2)cc1 ; c1ccc(-c2ccc3oc4cccc(-c5nc(-c6ccccc6)nc(-c6ccc7ccc8ccccc8c7c6)n5)c4c3c2)cc1 ; c1ccc(-c2cc(-c3nc(-c4ccccc4)nc(-c4ccc5ccc6ccccc6c5c4)n3)c3c(c2)oc2ccccc23)cc1]

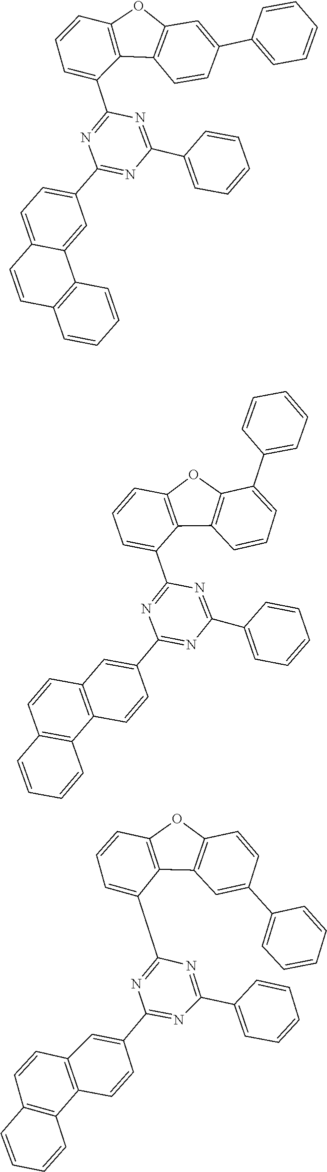 [SMILES: c1ccc(-c2ccc3c(c2)oc2cccc(-c4nc(-c5ccccc5)nc(-c5ccc6ccc7ccccc7c6c5)n4)c23)cc1 ; c1ccc(-c2nc(-c3ccc4c(ccc5ccccc54)c3)nc(-c3cccc4oc5c(-c6ccccc6)cccc5c34)n2)cc1 ; c1ccc(-c2ccc3oc4cccc(-c5nc(-c6ccccc6)nc(-c6ccc7c(ccc8ccccc87)c6)n5)c4c3c2)cc1]

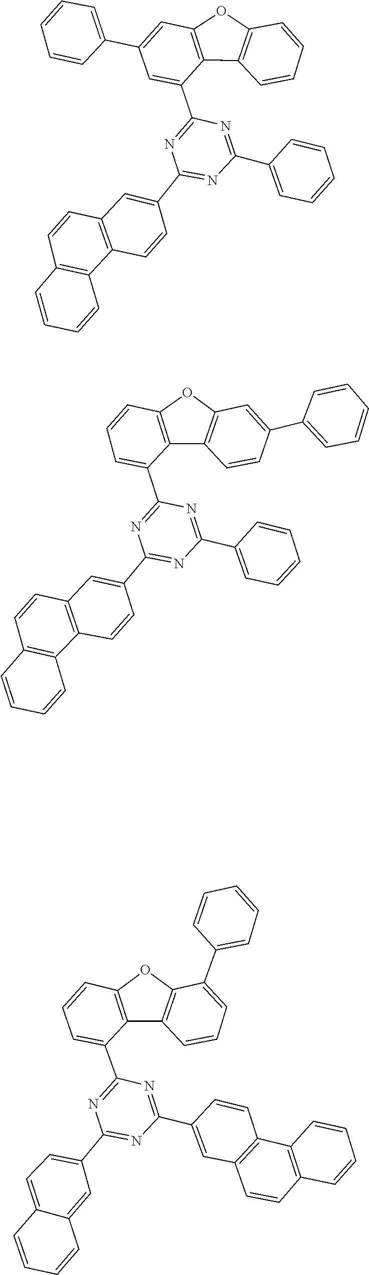 [SMILES: c1ccc(-c2cc(-c3nc(-c4ccccc4)nc(-c4ccc5c(ccc6ccccc65)c4)n3)c3c(c2)oc2ccccc23)cc1 ; c1ccc(-c2ccc3c(c2)oc2cccc(-c4nc(-c5ccccc5)nc(-c5ccc6c(ccc7ccccc76)c5)n4)c23)cc1 ; c1ccc(-c2cccc3c2oc2cccc(-c4nc(-c5ccc6ccccc6c5)nc(-c5ccc6c(ccc7ccccc76)c5)n4)c23)cc1]

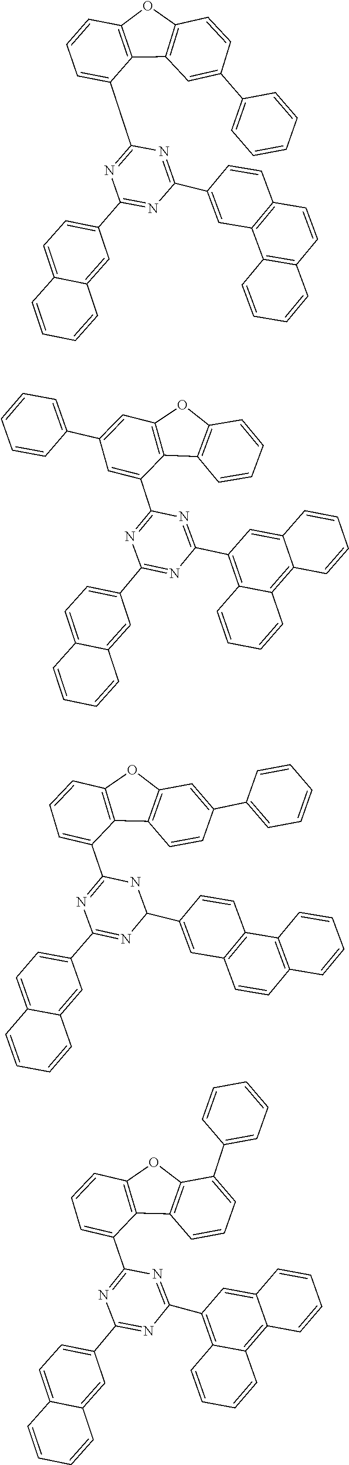

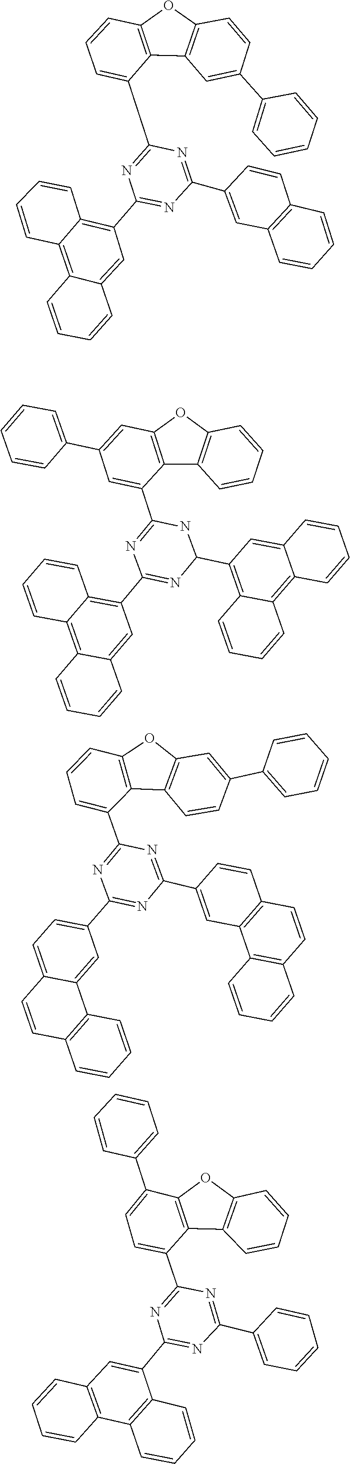

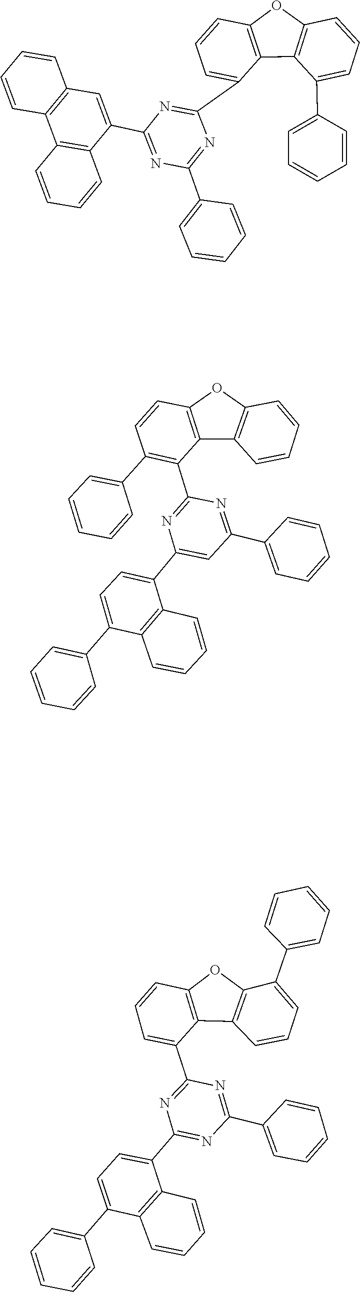 [SMILES: c1ccc(-c2nc(-c3cc4ccccc4c4ccccc34)nc(-c3cccc4oc5cccc(-c6ccccc6)c5c34)n2)cc1 ; c1ccc(-c2cc(-c3ccc(-c4ccccc4)c4ccccc34)nc(-c3c(-c4ccccc4)ccc4oc5ccccc5c34)n2)cc1 ; c1ccc(-c2nc(-c3ccc(-c4ccccc4)c4ccccc34)nc(-c3cccc4oc5c(-c6ccccc6)cccc5c34)n2)cc1]

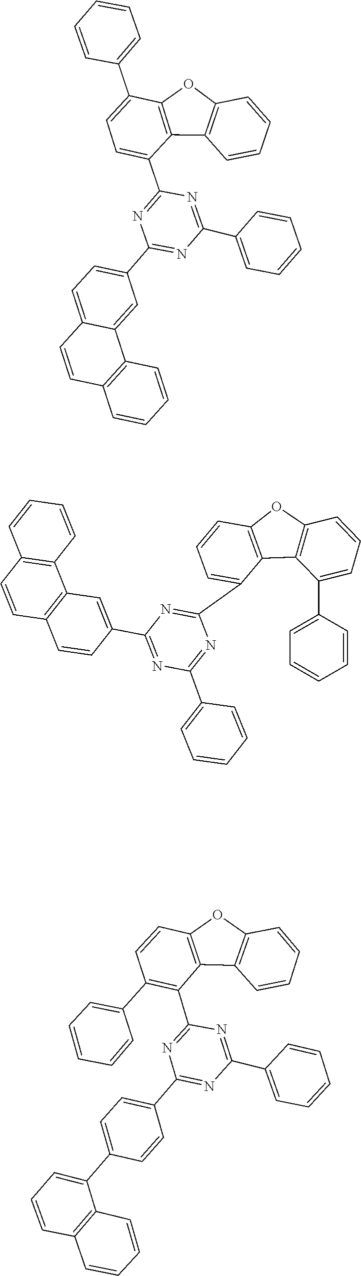 [SMILES: c1ccc(-c2nc(-c3ccc4ccc5ccccc5c4c3)nc(-c3ccc(-c4ccccc4)c4oc5ccccc5c34)n2)cc1 ; c1ccc(-c2nc(-c3ccc4ccc5ccccc5c4c3)nc(-c3cccc4oc5cccc(-c6ccccc6)c5c34)n2)cc1 ; c1ccc(-c2nc(-c3ccc(-c4cccc5ccccc45)cc3)nc(-c3c(-c4ccccc4)ccc4oc5ccccc5c34)n2)cc1]

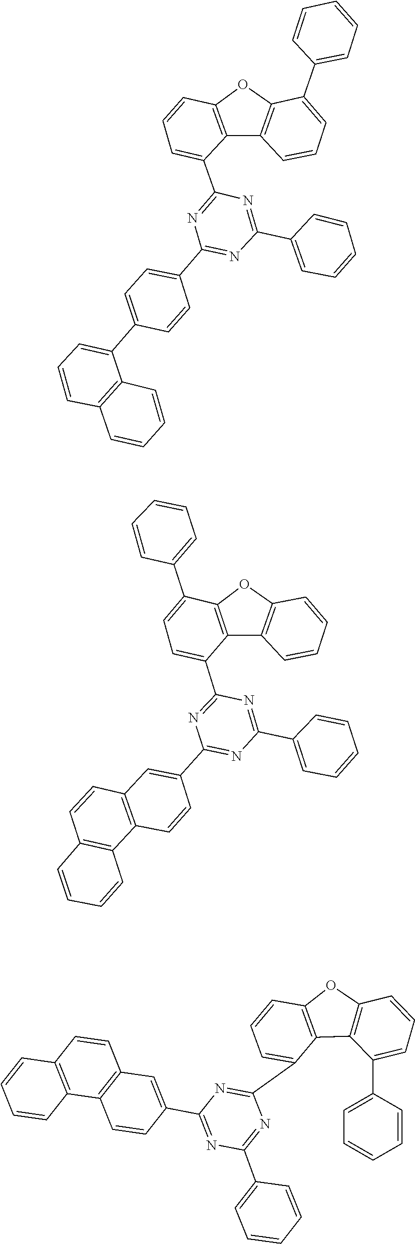 [SMILES: c1ccc(-c2nc(-c3ccc(-c4cccc5ccccc45)cc3)nc(-c3cccc4oc5c(-c6ccccc6)cccc5c34)n2)cc1 ; c1ccc(-c2nc(-c3ccc4c(ccc5ccccc54)c3)nc(-c3ccc(-c4ccccc4)c4oc5ccccc5c34)n2)cc1 ; c1ccc(-c2nc(-c3ccc4c(ccc5ccccc54)c3)nc(-c3cccc4oc5cccc(-c6ccccc6)c5c34)n2)cc1]

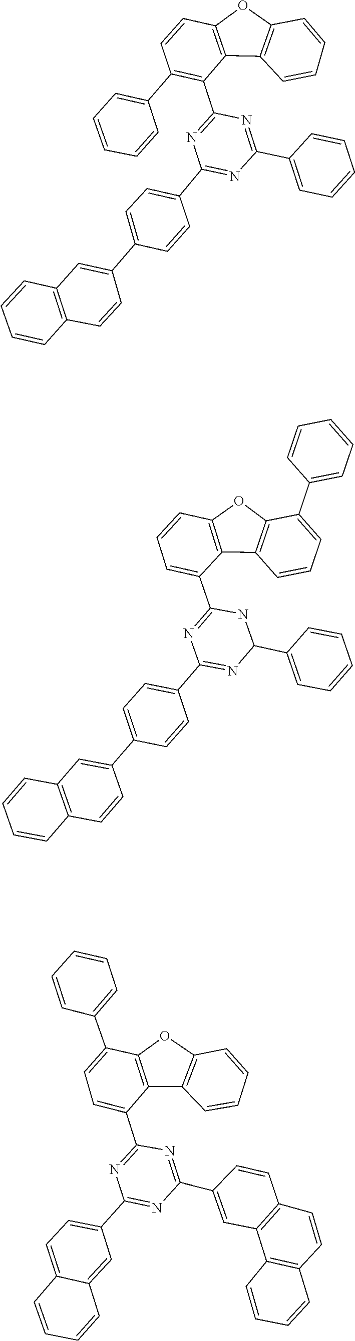

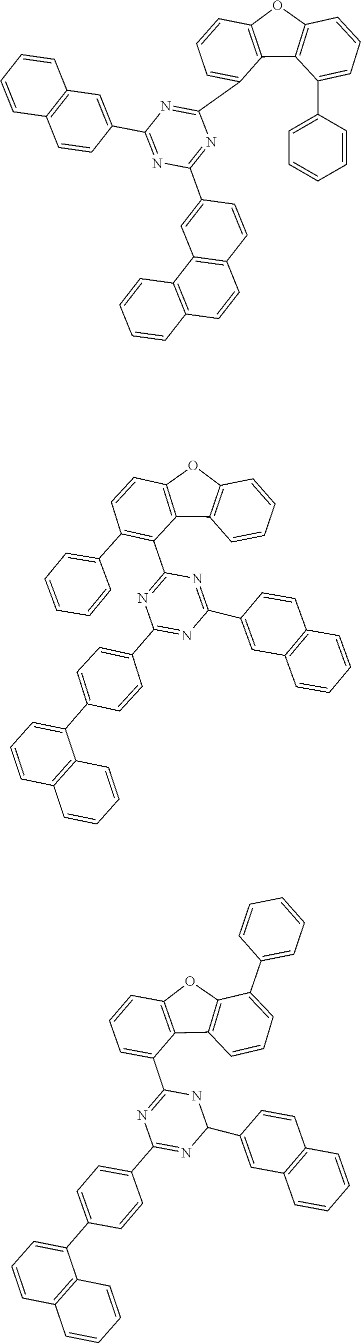

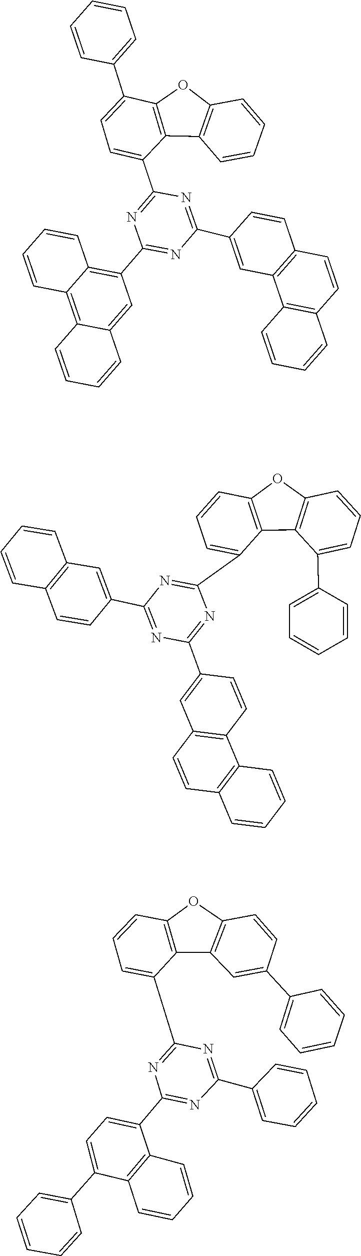 [SMILES: c1ccc(-c2ccc(-c3nc(-c4ccc5ccc6ccccc6c5c4)nc(-c4cc5ccccc5c5ccccc45)n3)c3c2oc2ccccc23)cc1 ; c1ccc(-c2cccc3oc4cccc(-c5nc(-c6ccc7ccccc7c6)nc(-c6ccc7c(ccc8ccccc87)c6)n5)c4c23)cc1 ; c1ccc(-c2ccc3oc4cccc(-c5nc(-c6ccccc6)nc(-c6ccc(-c7ccccc7)c7ccccc67)n5)c4c3c2)cc1]

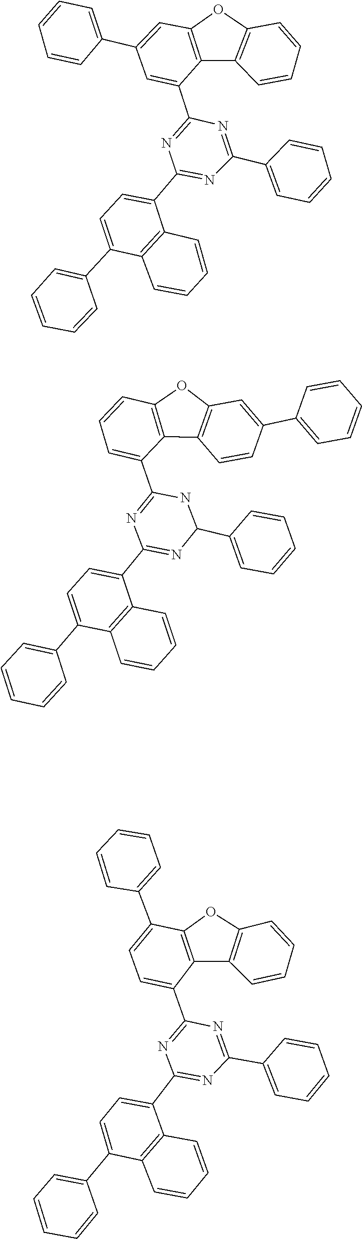

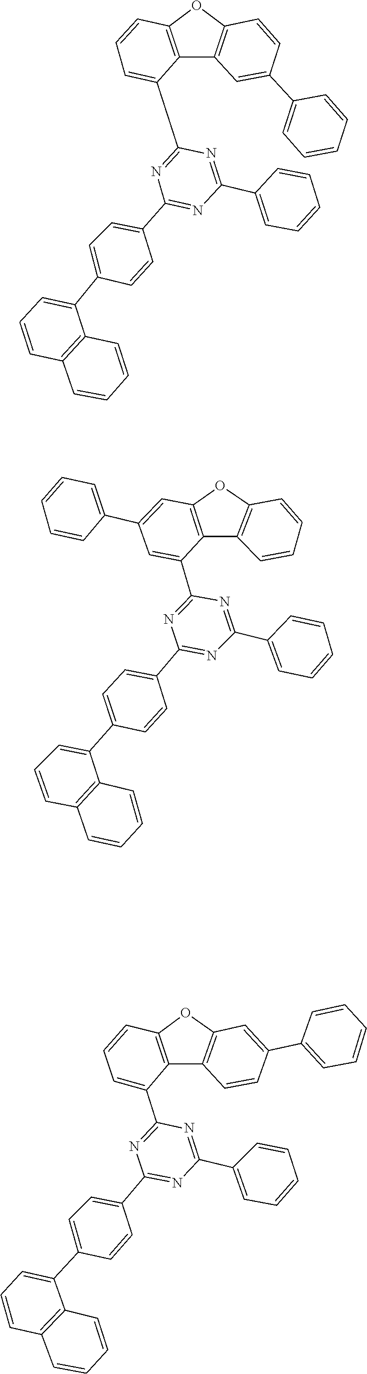 [SMILES: c1ccc(-c2ccc3oc4cccc(-c5nc(-c6ccccc6)nc(-c6ccc(-c7cccc8ccccc78)cc6)n5)c4c3c2)cc1 ; c1ccc(-c2cc(-c3nc(-c4ccccc4)nc(-c4ccc(-c5cccc6ccccc56)cc4)n3)c3c(c2)oc2ccccc23)cc1 ; c1ccc(-c2ccc3c(c2)oc2cccc(-c4nc(-c5ccccc5)nc(-c5ccc(-c6cccc7ccccc67)cc5)n4)c23)cc1]

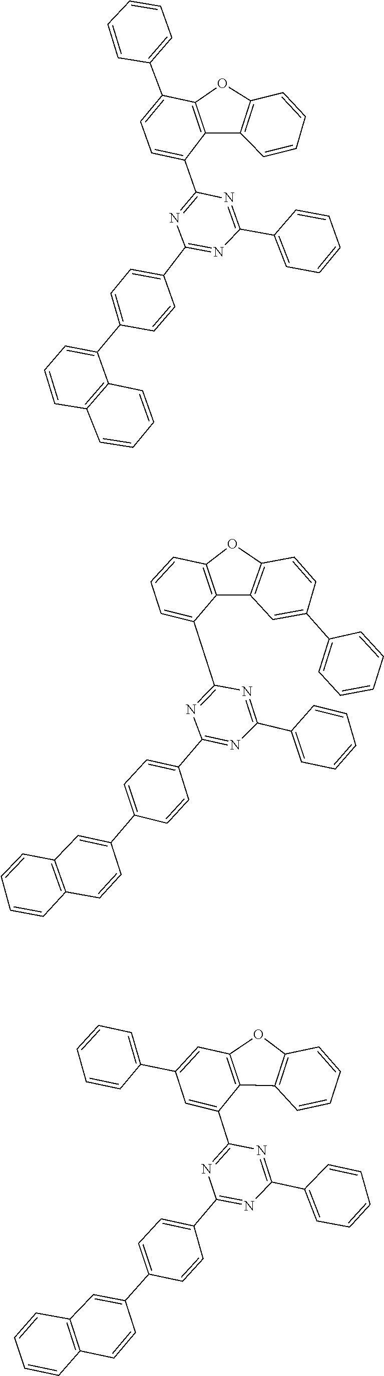 [SMILES: c1ccc(-c2nc(-c3ccc(-c4cccc5ccccc45)cc3)nc(-c3ccc(-c4ccccc4)c4oc5ccccc5c34)n2)cc1 ; c1ccc(-c2ccc3oc4cccc(-c5nc(-c6ccccc6)nc(-c6ccc(-c7ccc8ccccc8c7)cc6)n5)c4c3c2)cc1 ; c1ccc(-c2cc(-c3nc(-c4ccccc4)nc(-c4ccc(-c5ccc6ccccc6c5)cc4)n3)c3c(c2)oc2ccccc23)cc1]

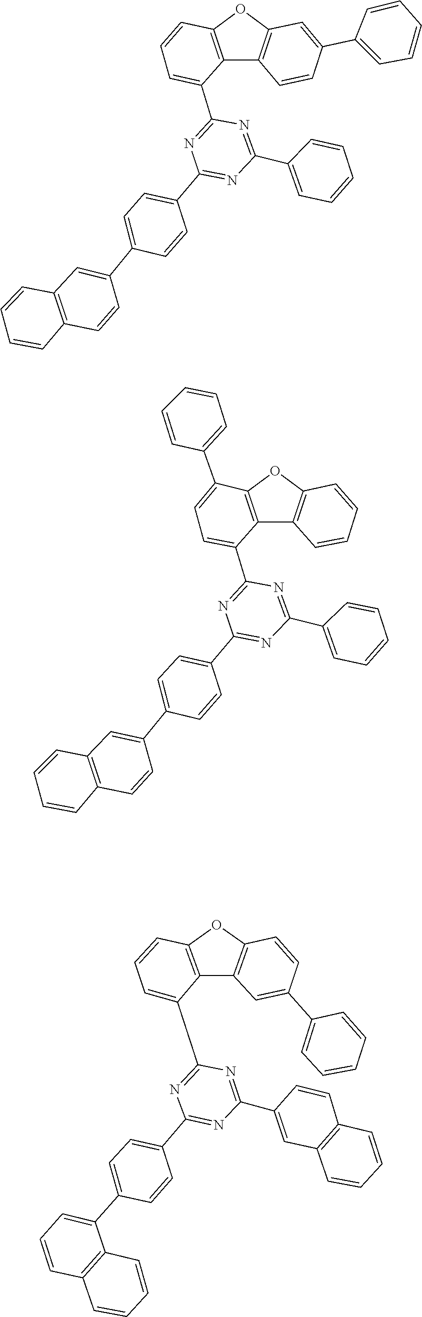 [SMILES: c1ccc(-c2ccc3c(c2)oc2cccc(-c4nc(-c5ccccc5)nc(-c5ccc(-c6ccc7ccccc7c6)cc5)n4)c23)cc1 ; c1ccc(-c2nc(-c3ccc(-c4ccc5ccccc5c4)cc3)nc(-c3ccc(-c4ccccc4)c4oc5ccccc5c34)n2)cc1 ; c1ccc(-c2ccc3oc4cccc(-c5nc(-c6ccc(-c7cccc8ccccc78)cc6)nc(-c6ccc7ccccc7c6)n5)c4c3c2)cc1]

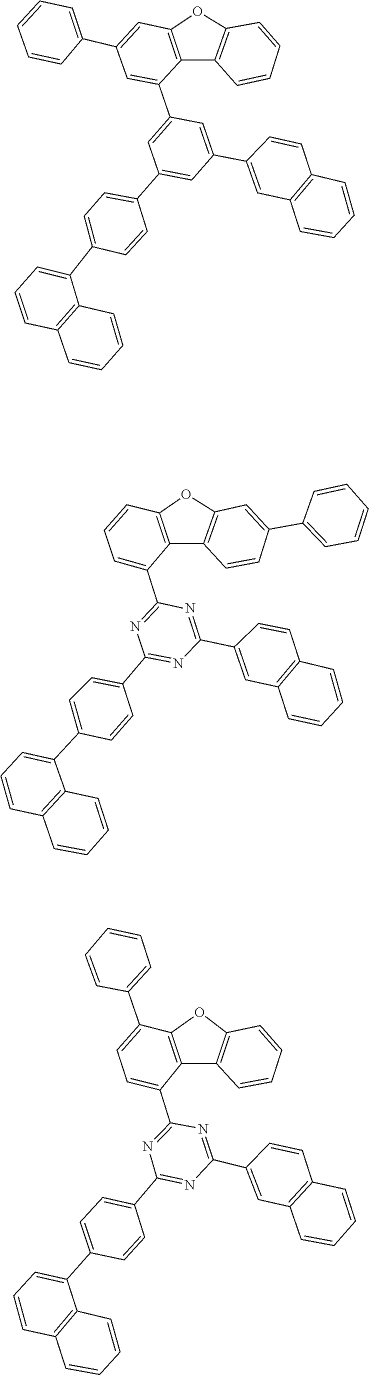 [SMILES: c1ccc(-c2cc(-c3cc(-c4ccc(-c5cccc6ccccc56)cc4)cc(-c4ccc5ccccc5c4)c3)c3c(c2)oc2ccccc23)cc1 ; c1ccc(-c2ccc3c(c2)oc2cccc(-c4nc(-c5ccc(-c6cccc7ccccc67)cc5)nc(-c5ccc6ccccc6c5)n4)c23)cc1 ; c1ccc(-c2ccc(-c3nc(-c4ccc(-c5cccc6ccccc56)cc4)nc(-c4ccc5ccccc5c4)n3)c3c2oc2ccccc23)cc1]

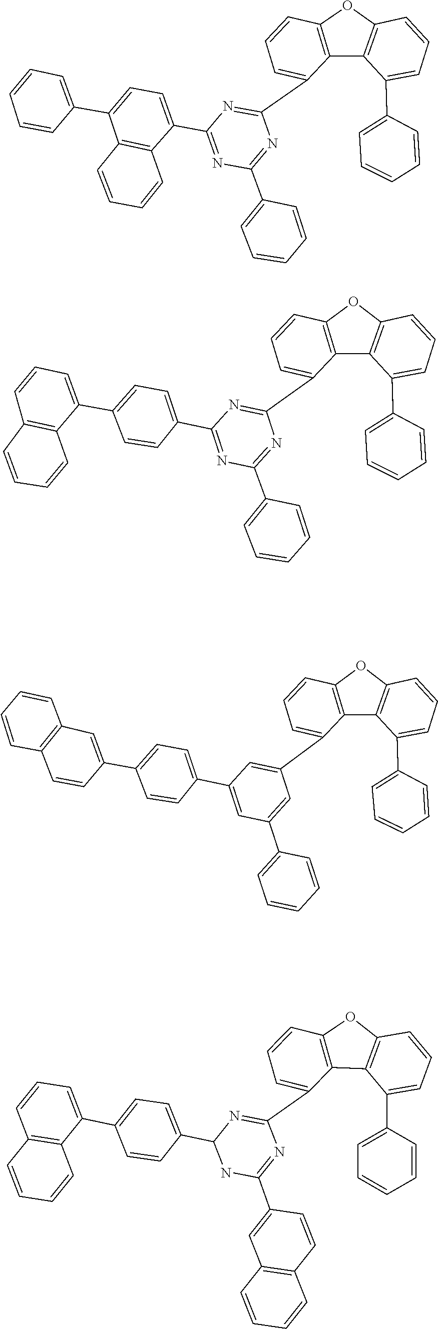

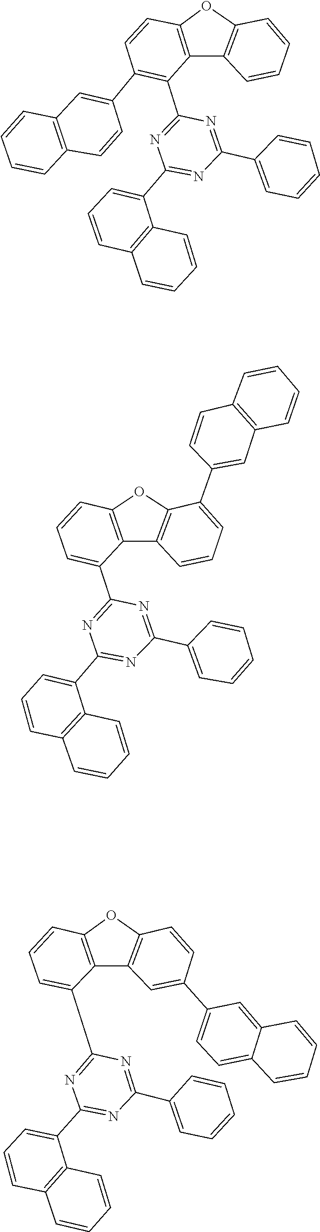 [SMILES: c1ccc(-c2nc(-c3cccc4ccccc34)nc(-c3c(-c4ccc5ccccc5c4)ccc4oc5ccccc5c34)n2)cc1 ; c1ccc(-c2nc(-c3cccc4ccccc34)nc(-c3cccc4oc5c(-c6ccc7ccccc7c6)cccc5c34)n2)cc1 ; c1ccc(-c2nc(-c3cccc4ccccc34)nc(-c3cccc4oc5ccc(-c6ccc7ccccc7c6)cc5c34)n2)cc1]

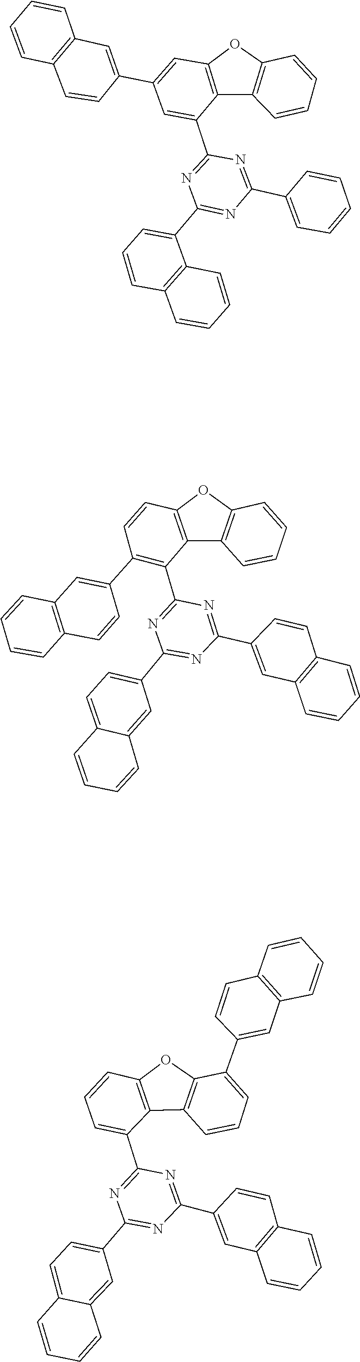 [SMILES: c1ccc(-c2nc(-c3cccc4ccccc34)nc(-c3cc(-c4ccc5ccccc5c4)cc4oc5ccccc5c34)n2)cc1 ; c1ccc2cc(-c3nc(-c4ccc5ccccc5c4)nc(-c4c(-c5ccc6ccccc6c5)ccc5oc6ccccc6c45)n3)ccc2c1 ; c1ccc2cc(-c3nc(-c4ccc5ccccc5c4)nc(-c4cccc5oc6c(-c7ccc8ccccc8c7)cccc6c45)n3)ccc2c1]

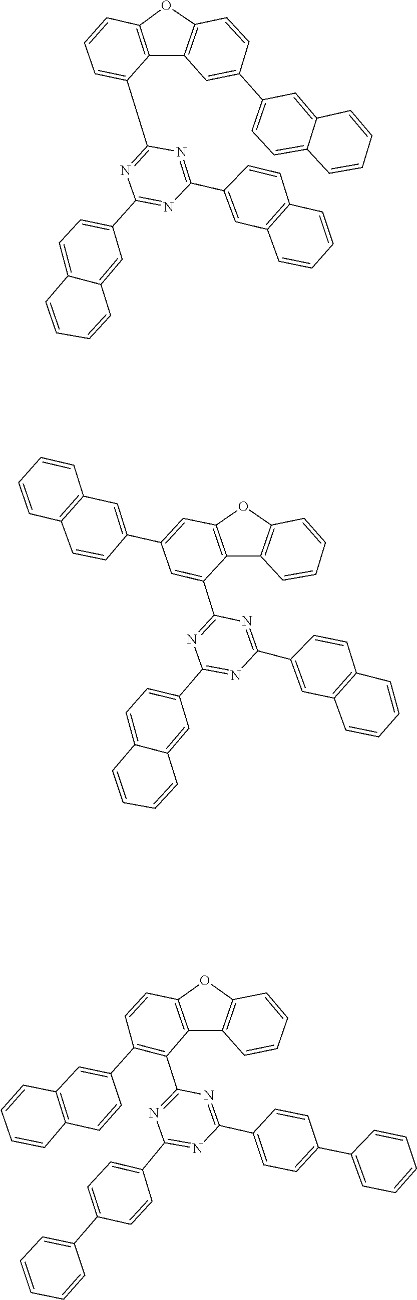 [SMILES: c1ccc2cc(-c3ccc4oc5cccc(-c6nc(-c7ccc8ccccc8c7)nc(-c7ccc8ccccc8c7)n6)c5c4c3)ccc2c1 ; c1ccc2cc(-c3cc(-c4nc(-c5ccc6ccccc6c5)nc(-c5ccc6ccccc6c5)n4)c4c(c3)oc3ccccc34)ccc2c1 ; c1ccc(-c2ccc(-c3nc(-c4ccc(-c5ccccc5)cc4)nc(-c4c(-c5ccc6ccccc6c5)ccc5oc6ccccc6c45)n3)cc2)cc1]

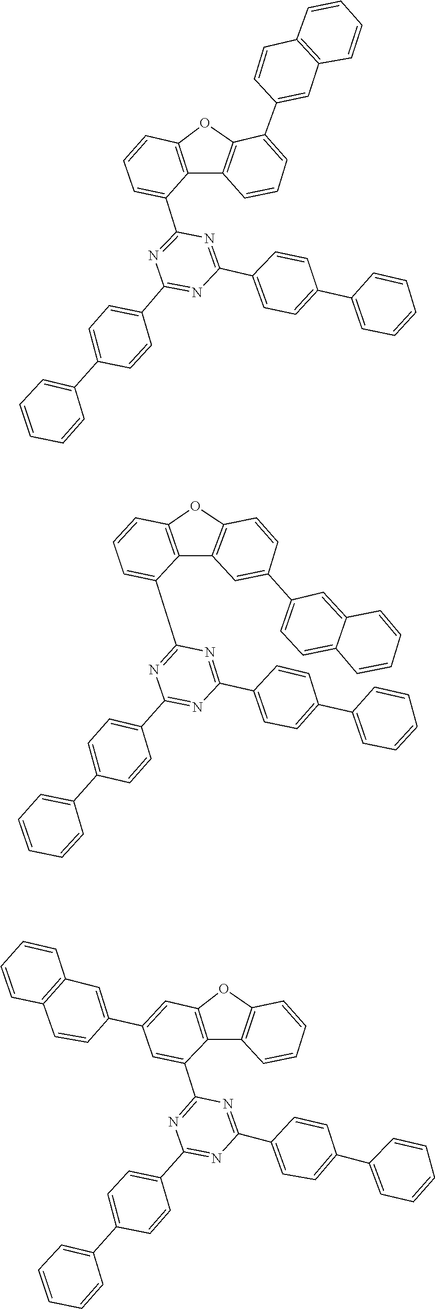 [SMILES: c1ccc(-c2ccc(-c3nc(-c4ccc(-c5ccccc5)cc4)nc(-c4cccc5oc6c(-c7ccc8ccccc8c7)cccc6c45)n3)cc2)cc1 ; c1ccc(-c2ccc(-c3nc(-c4ccc(-c5ccccc5)cc4)nc(-c4cccc5oc6ccc(-c7ccc8ccccc8c7)cc6c45)n3)cc2)cc1 ; c1ccc(-c2ccc(-c3nc(-c4ccc(-c5ccccc5)cc4)nc(-c4cc(-c5ccc6ccccc6c5)cc5oc6ccccc6c45)n3)cc2)cc1]

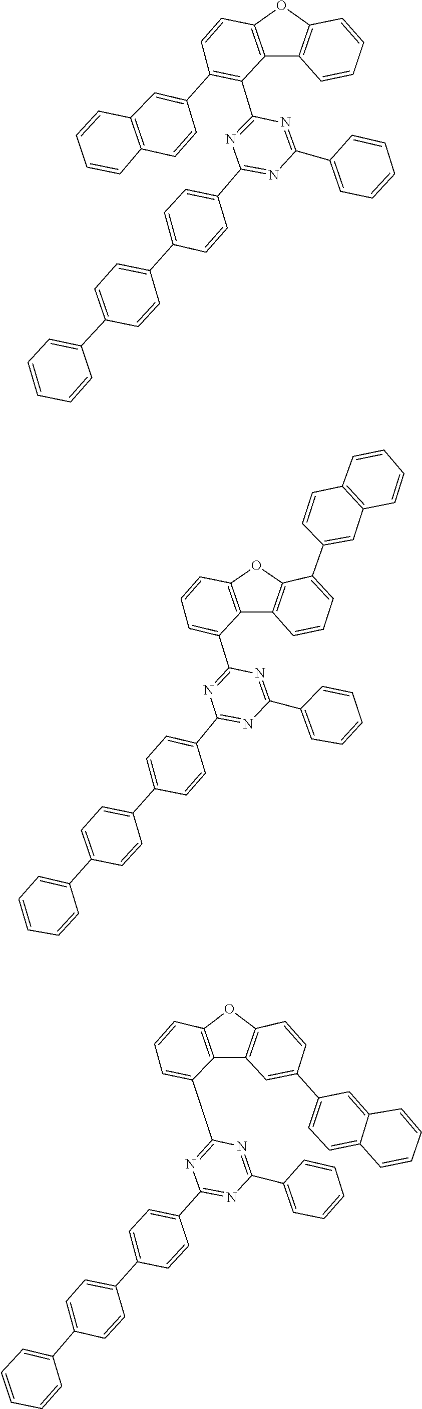 [SMILES: c1ccc(-c2ccc(-c3ccc(-c4nc(-c5ccccc5)nc(-c5c(-c6ccc7ccccc7c6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc9ccccc9c8)cccc7c56)n4)cc3)cc2)cc1 ; c1ccc(-c2ccc(-c3ccc(-c4nc(-c5ccccc5)nc(-c5cccc6oc7ccc(-c8ccc9ccccc9c8)cc7c56)n4)cc3)cc2)cc1]

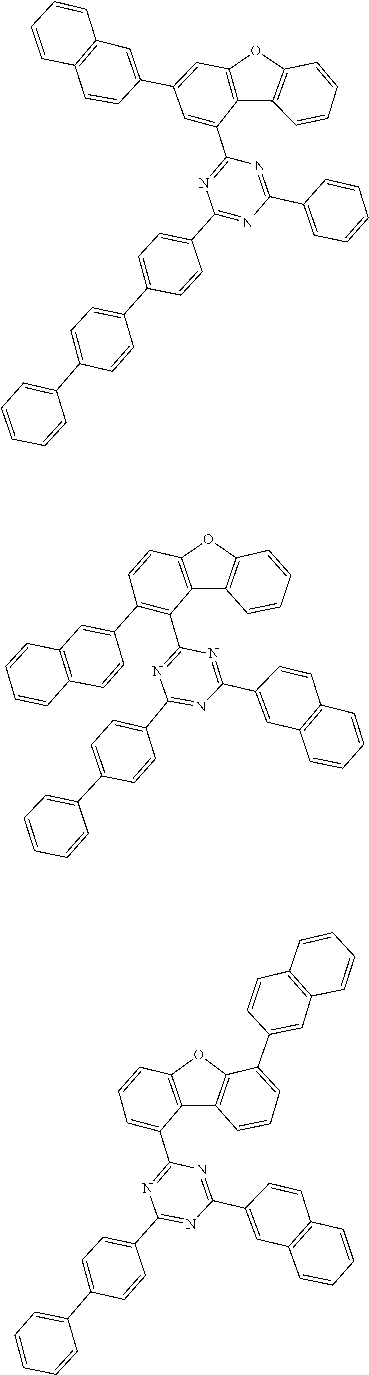 [SMILES: c1ccc(-c2ccc(-c3ccc(-c4nc(-c5ccccc5)nc(-c5cc(-c6ccc7ccccc7c6)cc6oc7ccccc7c56)n4)cc3)cc2)cc1 ; c1ccc(-c2ccc(-c3nc(-c4ccc5ccccc5c4)nc(-c4c(-c5ccc6ccccc6c5)ccc5oc6ccccc6c45)n3)cc2)cc1 ; c1ccc(-c2ccc(-c3nc(-c4ccc5ccccc5c4)nc(-c4cccc5oc6c(-c7ccc8ccccc8c7)cccc6c45)n3)cc2)cc1]

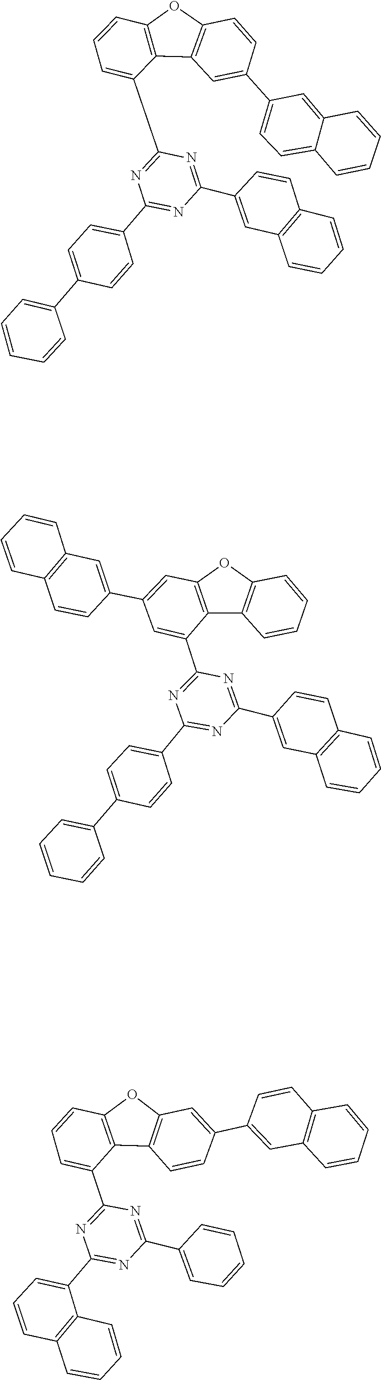 [SMILES: c1ccc(-c2ccc(-c3nc(-c4ccc5ccccc5c4)nc(-c4cccc5oc6ccc(-c7ccc8ccccc8c7)cc6c45)n3)cc2)cc1 ; c1ccc(-c2ccc(-c3nc(-c4ccc5ccccc5c4)nc(-c4cc(-c5ccc6ccccc6c5)cc5oc6ccccc6c45)n3)cc2)cc1 ; c1ccc(-c2nc(-c3cccc4ccccc34)nc(-c3cccc4oc5cc(-c6ccc7ccccc7c6)ccc5c34)n2)cc1]

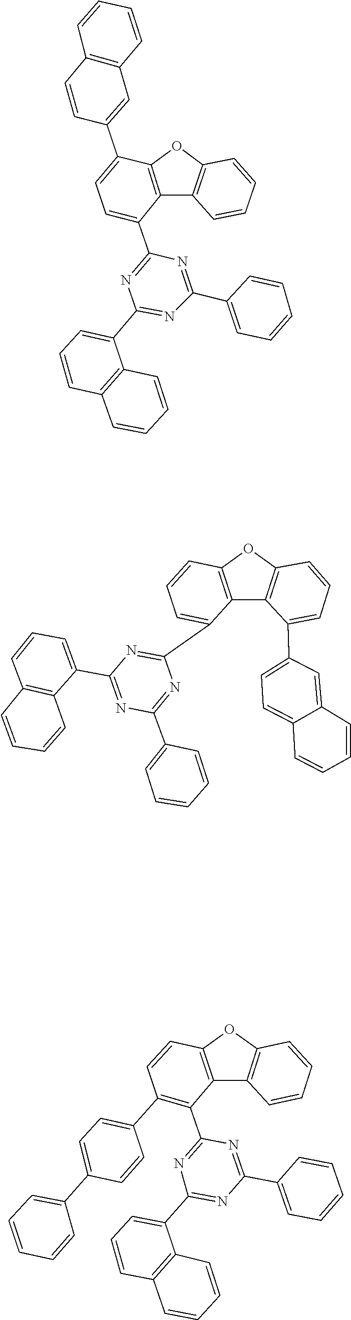 [SMILES: c1ccc(-c2nc(-c3cccc4ccccc34)nc(-c3ccc(-c4ccc5ccccc5c4)c4oc5ccccc5c34)n2)cc1 ; c1ccc(-c2nc(-c3cccc4ccccc34)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccccc4)nc(-c4cccc5ccccc45)n3)cc2)cc1]

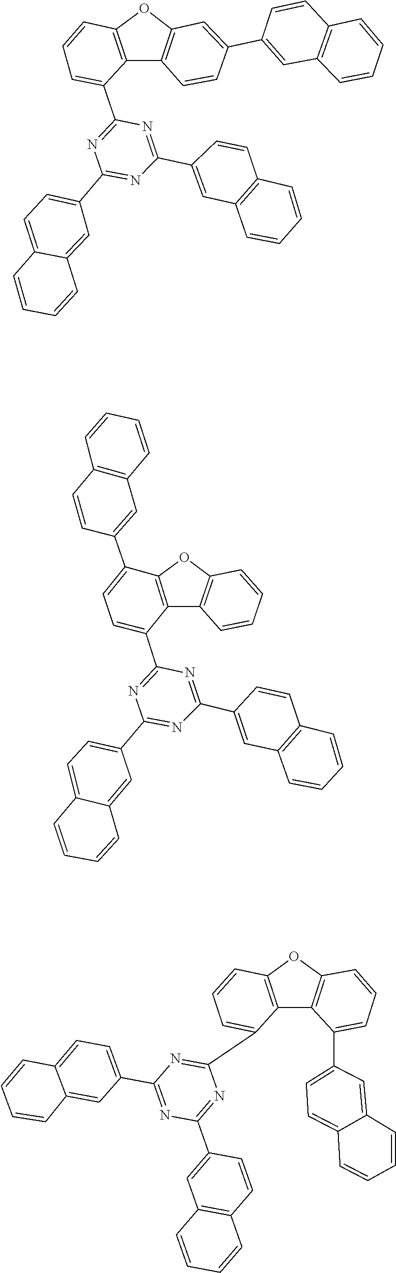 [SMILES: c1ccc2cc(-c3ccc4c(c3)oc3cccc(-c5nc(-c6ccc7ccccc7c6)nc(-c6ccc7ccccc7c6)n5)c34)ccc2c1 ; c1ccc2cc(-c3nc(-c4ccc5ccccc5c4)nc(-c4ccc(-c5ccc6ccccc6c5)c5oc6ccccc6c45)n3)ccc2c1 ; c1ccc2cc(-c3nc(-c4ccc5ccccc5c4)nc(-c4cccc5oc6cccc(-c7ccc8ccccc8c7)c6c45)n3)ccc2c1]

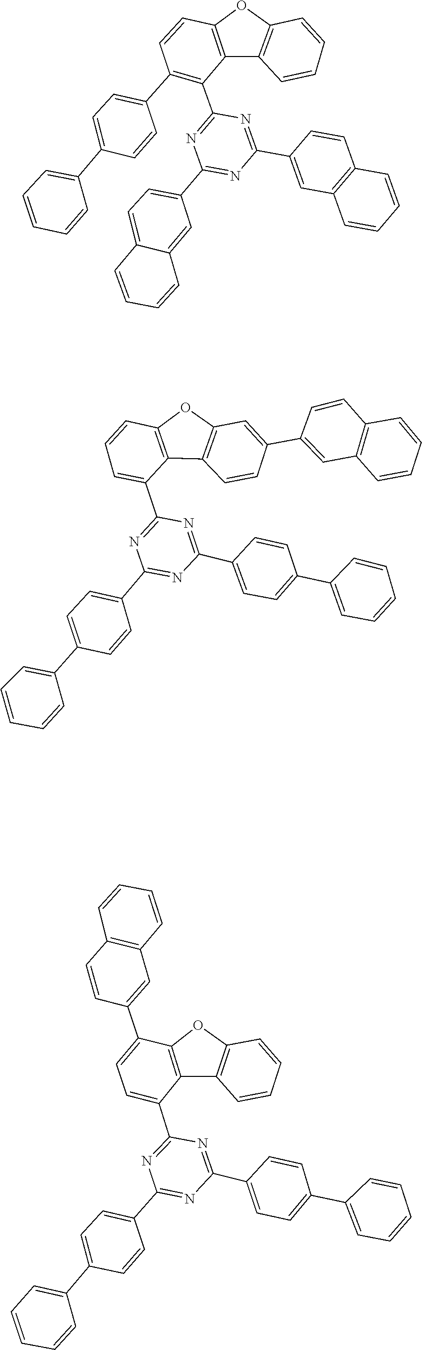 [SMILES: c1ccc(-c2ccc(-c3ccc4oc5ccccc5c4c3-c3nc(-c4ccc5ccccc5c4)nc(-c4ccc5ccccc5c4)n3)cc2)cc1 ; c1ccc(-c2ccc(-c3nc(-c4ccc(-c5ccccc5)cc4)nc(-c4cccc5oc6cc(-c7ccc8ccccc8c7)ccc6c45)n3)cc2)cc1 ; c1ccc(-c2ccc(-c3nc(-c4ccc(-c5ccccc5)cc4)nc(-c4ccc(-c5ccc6ccccc6c5)c5oc6ccccc6c45)n3)cc2)cc1]

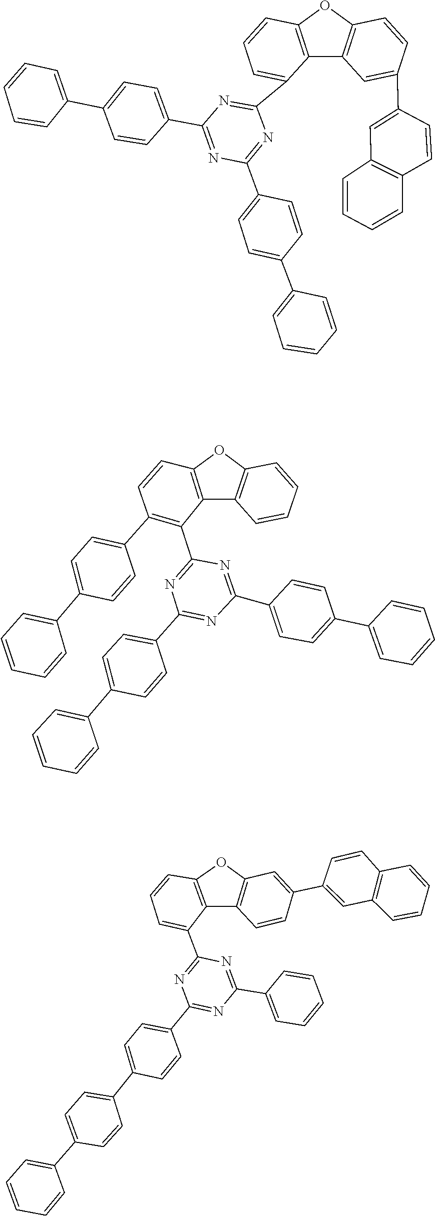 [SMILES: c1ccc(-c2ccc(-c3nc(-c4ccc(-c5ccccc5)cc4)nc(-c4cccc5oc6ccc(-c7ccc8ccccc8c7)cc6c45)n3)cc2)cc1 ; c1ccc(-c2ccc(-c3nc(-c4ccc(-c5ccccc5)cc4)nc(-c4c(-c5ccc(-c6ccccc6)cc5)ccc5oc6ccccc6c45)n3)cc2)cc1 ; c1ccc(-c2ccc(-c3ccc(-c4nc(-c5ccccc5)nc(-c5cccc6oc7cc(-c8ccc9ccccc9c8)ccc7c56)n4)cc3)cc2)cc1]

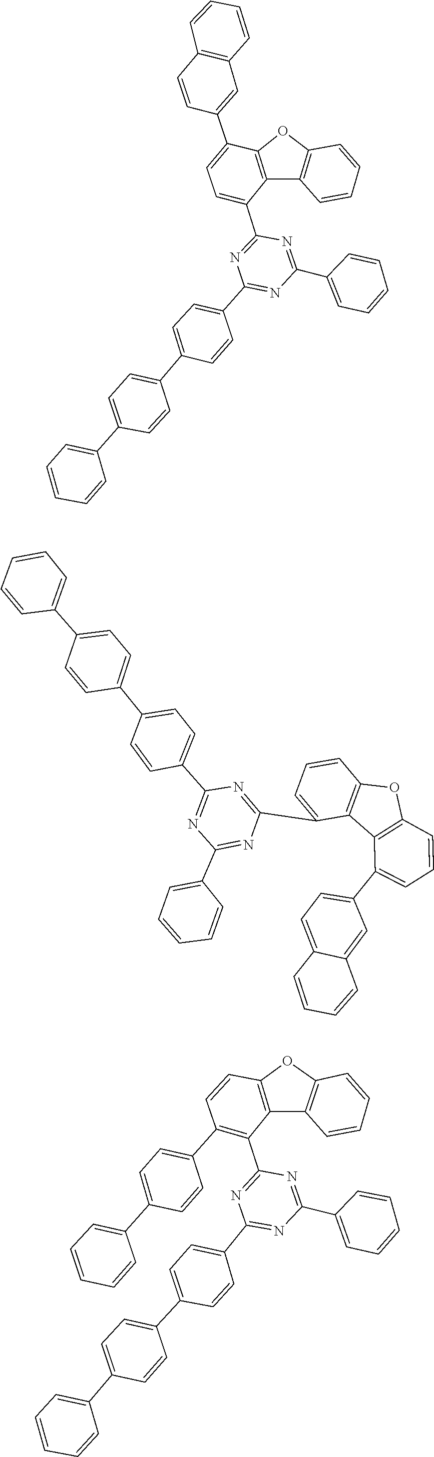 [SMILES: c1ccc(-c2ccc(-c3ccc(-c4nc(-c5ccccc5)nc(-c5ccc(-c6ccc7ccccc7c6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9ccccc9c8)c7c56)n4)cc3)cc2)cc1 ; c1ccc(-c2ccc(-c3ccc(-c4nc(-c5ccccc5)nc(-c5c(-c6ccc(-c7ccccc7)cc6)ccc6oc7ccccc7c56)n4)cc3)cc2)cc1]

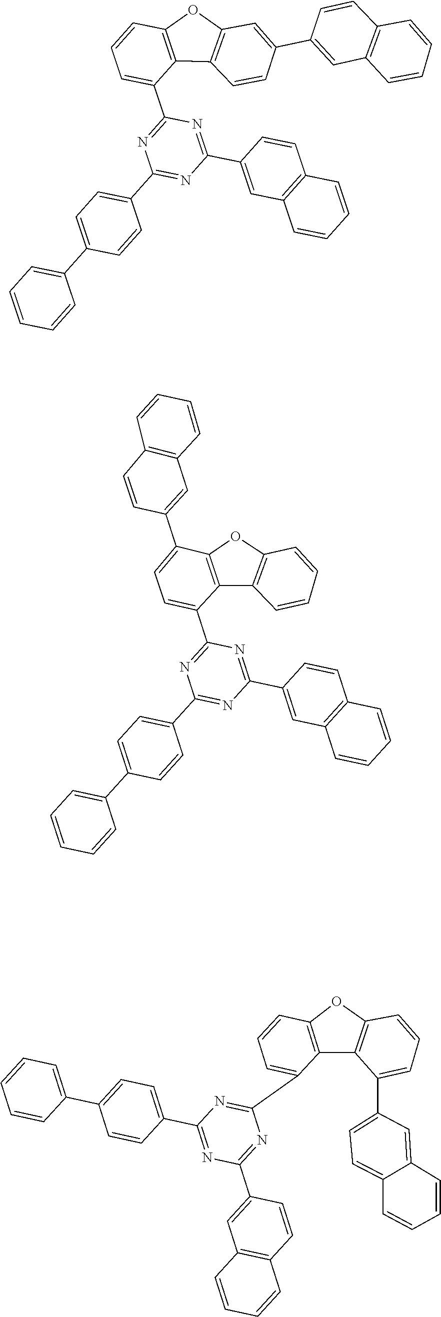 [SMILES: c1ccc(-c2ccc(-c3nc(-c4ccc5ccccc5c4)nc(-c4cccc5oc6cc(-c7ccc8ccccc8c7)ccc6c45)n3)cc2)cc1 ; c1ccc(-c2ccc(-c3nc(-c4ccc5ccccc5c4)nc(-c4ccc(-c5ccc6ccccc6c5)c5oc6ccccc6c45)n3)cc2)cc1 ; c1ccc(-c2ccc(-c3nc(-c4ccc5ccccc5c4)nc(-c4cccc5oc6cccc(-c7ccc8ccccc8c7)c6c45)n3)cc2)cc1]

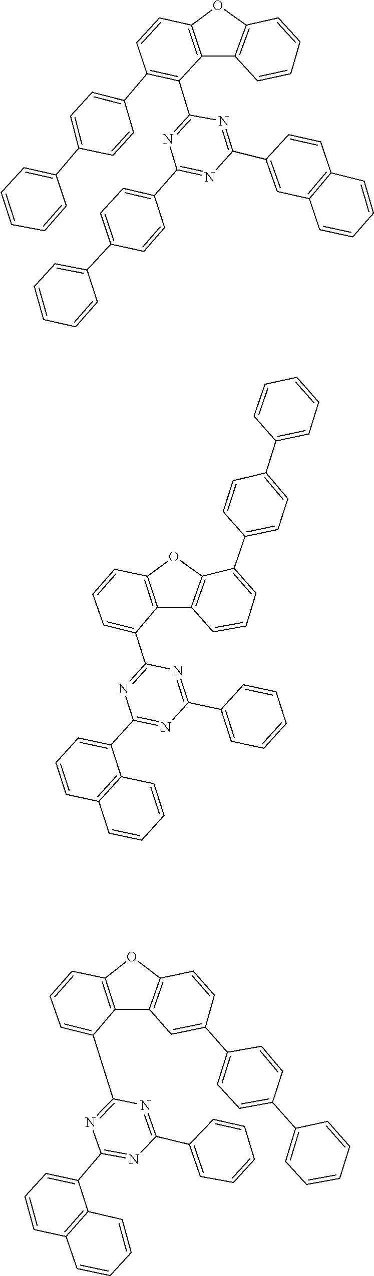 [SMILES: c1ccc(-c2ccc(-c3nc(-c4ccc5ccccc5c4)nc(-c4c(-c5ccc(-c6ccccc6)cc5)ccc5oc6ccccc6c45)n3)cc2)cc1 ; c1ccc(-c2ccc(-c3cccc4c3oc3cccc(-c5nc(-c6ccccc6)nc(-c6cccc7ccccc67)n5)c34)cc2)cc1 ; c1ccc(-c2ccc(-c3ccc4oc5cccc(-c6nc(-c7ccccc7)nc(-c7cccc8ccccc78)n6)c5c4c3)cc2)cc1]

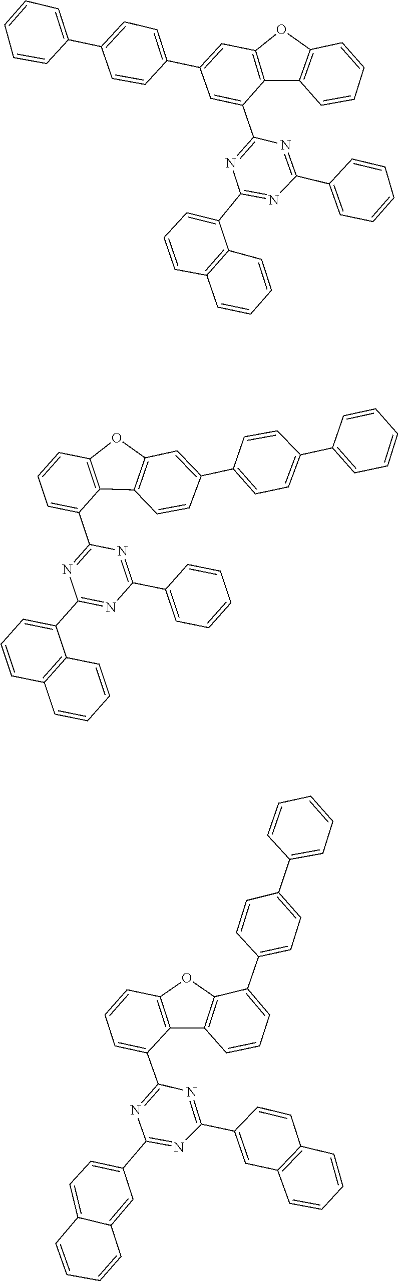 [SMILES: c1ccc(-c2ccc(-c3cc(-c4nc(-c5ccccc5)nc(-c5cccc6ccccc56)n4)c4c(c3)oc3ccccc34)cc2)cc1 ; c1ccc(-c2ccc(-c3ccc4c(c3)oc3cccc(-c5nc(-c6ccccc6)nc(-c6cccc7ccccc67)n5)c34)cc2)cc1 ; c1ccc(-c2ccc(-c3cccc4c3oc3cccc(-c5nc(-c6ccc7ccccc7c6)nc(-c6ccc7ccccc7c6)n5)c34)cc2)cc1]

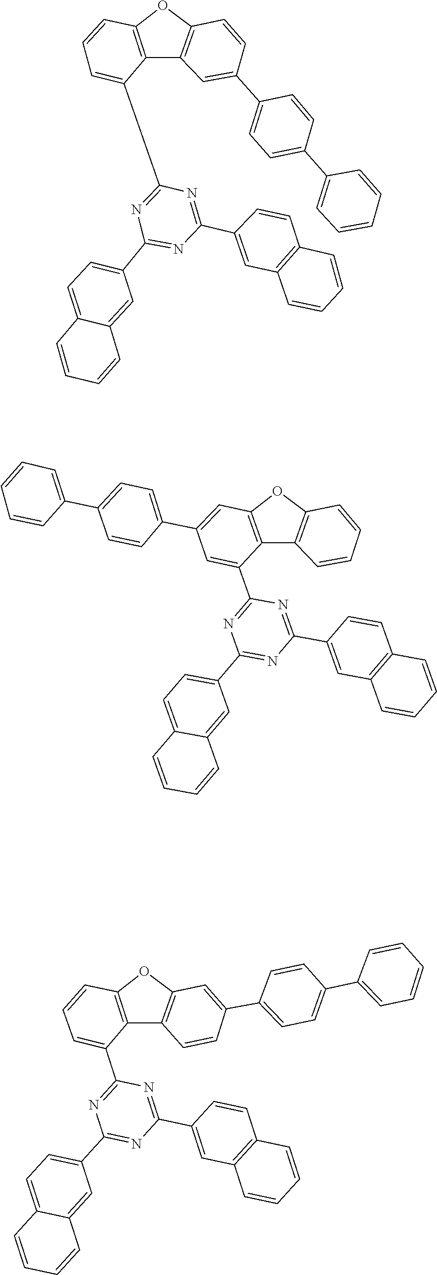 [SMILES: c1ccc(-c2ccc(-c3ccc4oc5cccc(-c6nc(-c7ccc8ccccc8c7)nc(-c7ccc8ccccc8c7)n6)c5c4c3)cc2)cc1 ; c1ccc(-c2ccc(-c3cc(-c4nc(-c5ccc6ccccc6c5)nc(-c5ccc6ccccc6c5)n4)c4c(c3)oc3ccccc34)cc2)cc1 ; c1ccc(-c2ccc(-c3ccc4c(c3)oc3cccc(-c5nc(-c6ccc7ccccc7c6)nc(-c6ccc7ccccc7c6)n5)c34)cc2)cc1]

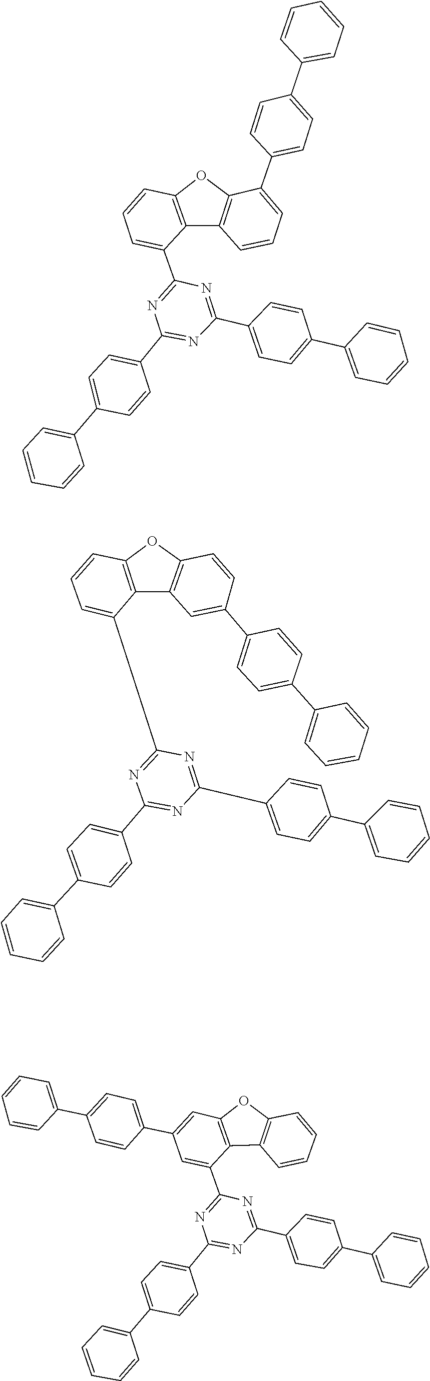 [SMILES: c1ccc(-c2ccc(-c3nc(-c4ccc(-c5ccccc5)cc4)nc(-c4cccc5oc6c(-c7ccc(-c8ccccc8)cc7)cccc6c45)n3)cc2)cc1 ; c1ccc(-c2ccc(-c3ccc4oc5cccc(-c6nc(-c7ccc(-c8ccccc8)cc7)nc(-c7ccc(-c8ccccc8)cc7)n6)c5c4c3)cc2)cc1 ; c1ccc(-c2ccc(-c3cc(-c4nc(-c5ccc(-c6ccccc6)cc5)nc(-c5ccc(-c6ccccc6)cc5)n4)c4c(c3)oc3ccccc34)cc2)cc1]

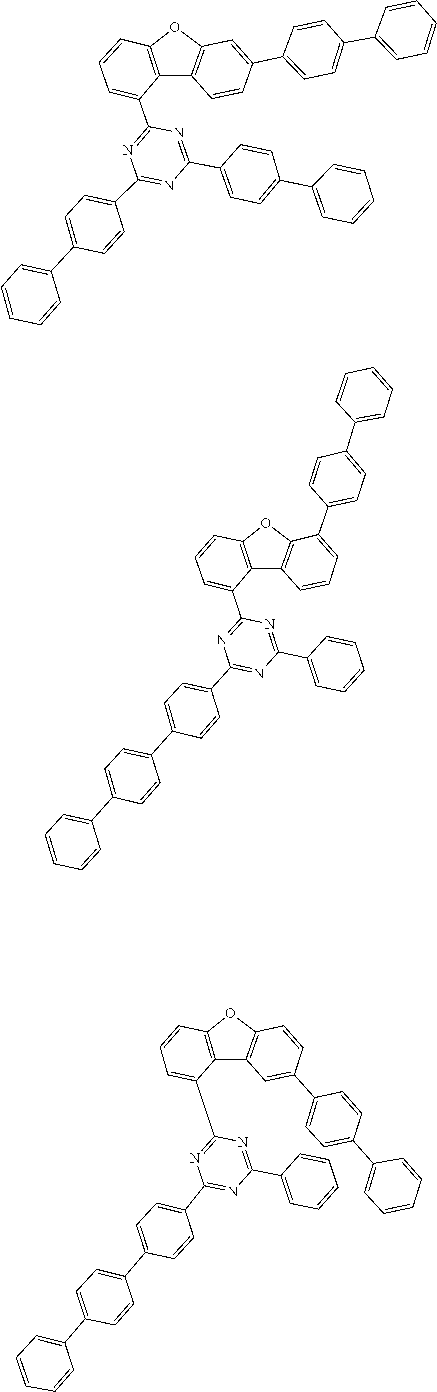 [SMILES: c1ccc(-c2ccc(-c3ccc4c(c3)oc3cccc(-c5nc(-c6ccc(-c7ccccc7)cc6)nc(-c6ccc(-c7ccccc7)cc6)n5)c34)cc2)cc1 ; c1ccc(-c2ccc(-c3ccc(-c4nc(-c5ccccc5)nc(-c5cccc6oc7c(-c8ccc(-c9ccccc9)cc8)cccc7c56)n4)cc3)cc2)cc1 ; c1ccc(-c2ccc(-c3ccc(-c4nc(-c5ccccc5)nc(-c5cccc6oc7ccc(-c8ccc(-c9ccccc9)cc8)cc7c56)n4)cc3)cc2)cc1]

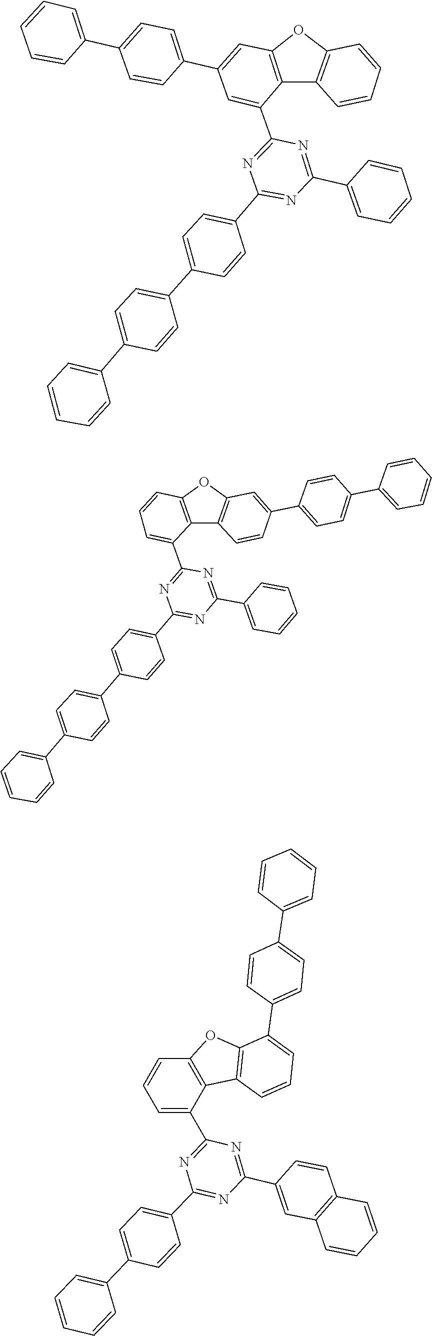 [SMILES: c1ccc(-c2ccc(-c3ccc(-c4nc(-c5ccccc5)nc(-c5cc(-c6ccc(-c7ccccc7)cc6)cc6oc7ccccc7c56)n4)cc3)cc2)cc1 ; c1ccc(-c2ccc(-c3ccc(-c4nc(-c5ccccc5)nc(-c5cccc6oc7cc(-c8ccc(-c9ccccc9)cc8)ccc7c56)n4)cc3)cc2)cc1 ; c1ccc(-c2ccc(-c3nc(-c4ccc5ccccc5c4)nc(-c4cccc5oc6c(-c7ccc(-c8ccccc8)cc7)cccc6c45)n3)cc2)cc1]

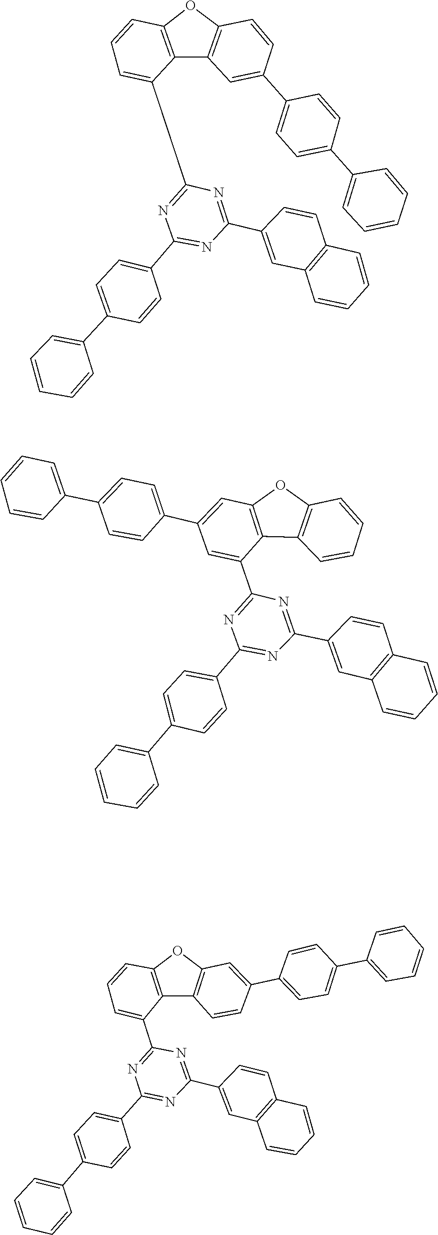 [SMILES: c1ccc(-c2ccc(-c3ccc4oc5cccc(-c6nc(-c7ccc(-c8ccccc8)cc7)nc(-c7ccc8ccccc8c7)n6)c5c4c3)cc2)cc1 ; c1ccc(-c2ccc(-c3cc(-c4nc(-c5ccc(-c6ccccc6)cc5)nc(-c5ccc6ccccc6c5)n4)c4c(c3)oc3ccccc34)cc2)cc1 ; c1ccc(-c2ccc(-c3ccc4c(c3)oc3cccc(-c5nc(-c6ccc(-c7ccccc7)cc6)nc(-c6ccc7ccccc7c6)n5)c34)cc2)cc1]

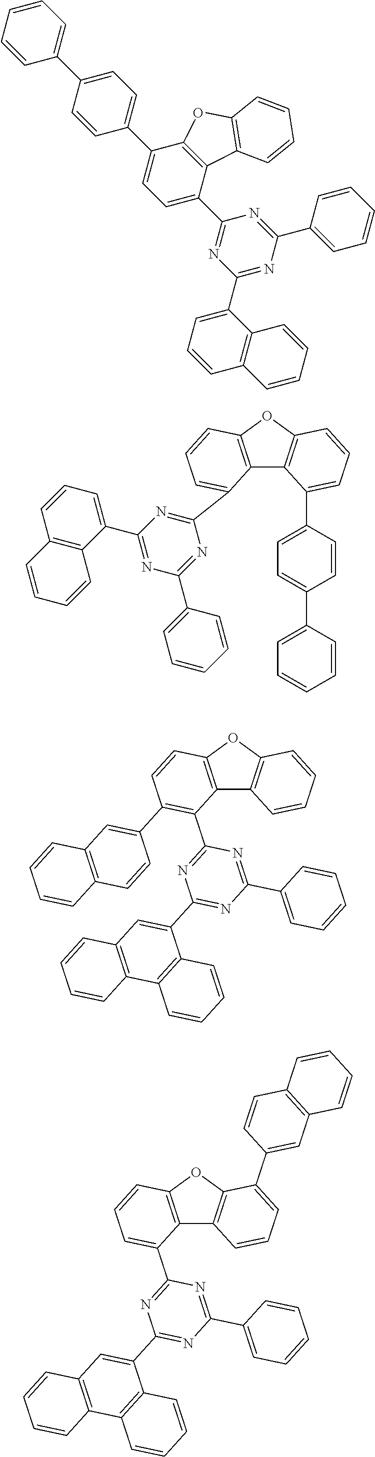

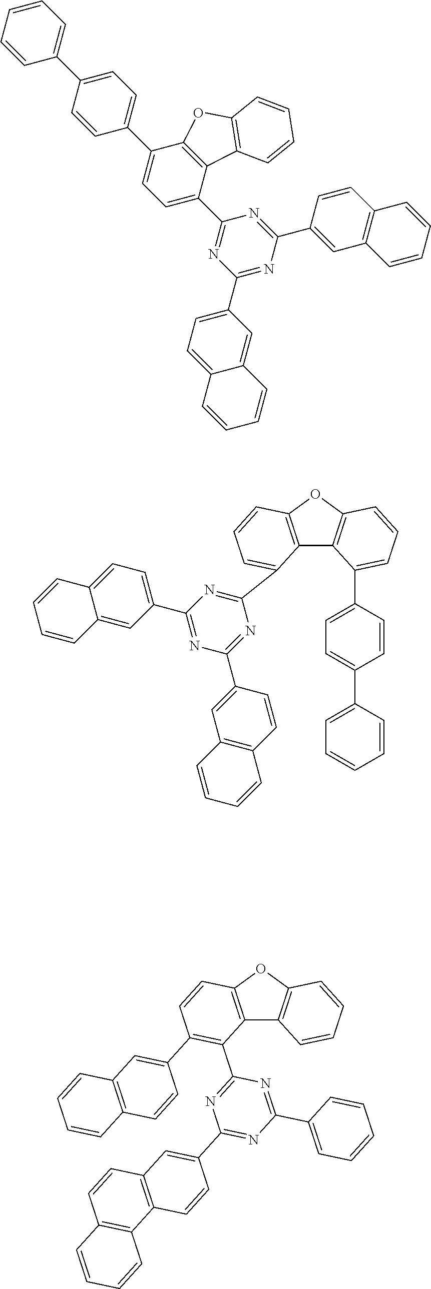 [SMILES: c1ccc(-c2ccc(-c3ccc(-c4nc(-c5ccc6ccccc6c5)nc(-c5ccc6ccccc6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccccc8c7)n6)c5c34)cc2)cc1 ; c1ccc(-c2nc(-c3ccc4c(ccc5ccccc54)c3)nc(-c3c(-c4ccc5ccccc5c4)ccc4oc5ccccc5c34)n2)cc1]

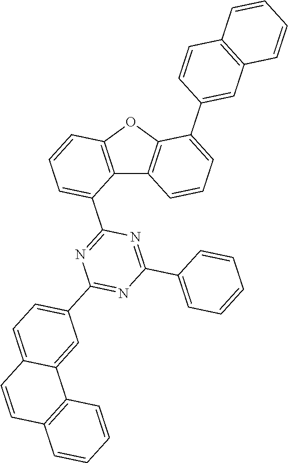 [SMILES: c1ccc(-c2nc(-c3ccc4ccc5ccccc5c4c3)nc(-c3cccc4oc5c(-c6ccc7ccccc7c6)cccc5c34)n2)cc1]

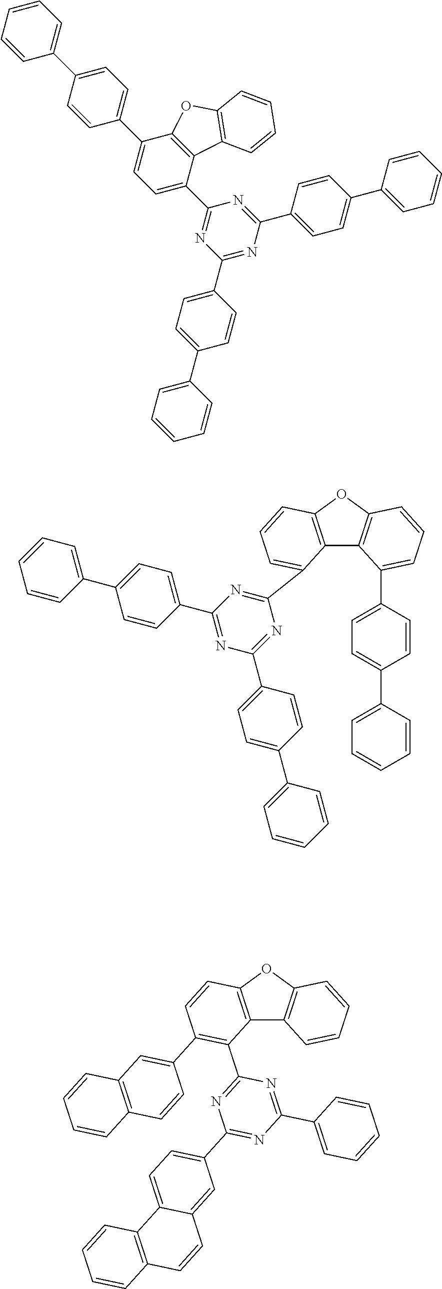 [SMILES: c1ccc(-c2ccc(-c3nc(-c4ccc(-c5ccccc5)cc4)nc(-c4ccc(-c5ccc(-c6ccccc6)cc5)c5oc6ccccc6c45)n3)cc2)cc1 ; c1ccc(-c2ccc(-c3nc(-c4ccc(-c5ccccc5)cc4)nc(-c4cccc5oc6cccc(-c7ccc(-c8ccccc8)cc7)c6c45)n3)cc2)cc1 ; c1ccc(-c2nc(-c3ccc4c(ccc5ccccc54)c3)nc(-c3c(-c4ccc5ccccc5c4)ccc4oc5ccccc5c34)n2)cc1]

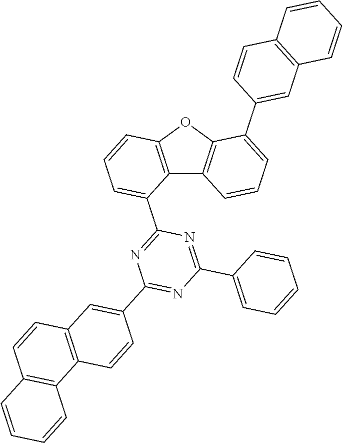 [SMILES: c1ccc(-c2nc(-c3ccc4c(ccc5ccccc54)c3)nc(-c3cccc4oc5c(-c6ccc7ccccc7c6)cccc5c34)n2)cc1]

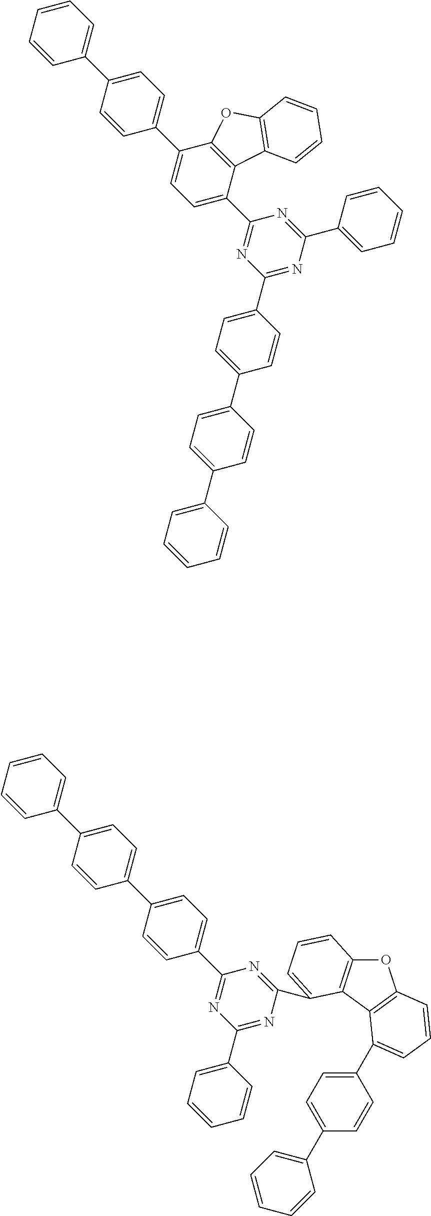 [SMILES: c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1]

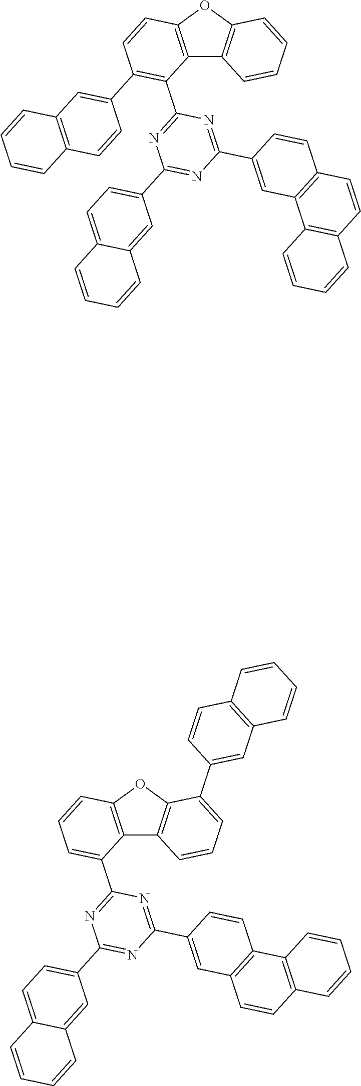 [SMILES: c1ccc2cc(-c3nc(-c4ccc5ccc6ccccc6c5c4)nc(-c4c(-c5ccc6ccccc6c5)ccc5oc6ccccc6c45)n3)ccc2c1 ; c1ccc2cc(-c3nc(-c4ccc5c(ccc6ccccc65)c4)nc(-c4cccc5oc6c(-c7ccc8ccccc8c7)cccc6c45)n3)ccc2c1]

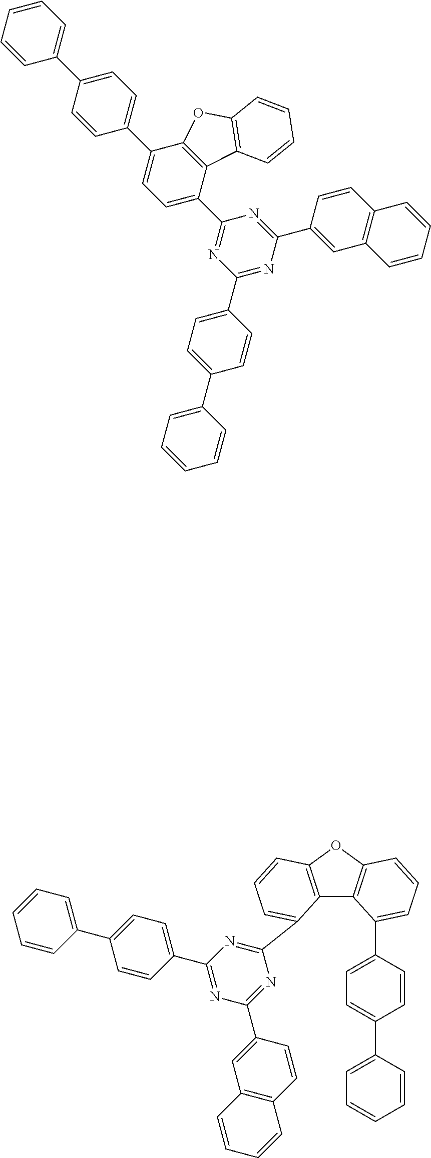 [SMILES: c1ccc(-c2ccc(-c3nc(-c4ccc5ccccc5c4)nc(-c4ccc(-c5ccc(-c6ccccc6)cc5)c5oc6ccccc6c45)n3)cc2)cc1 ; c1ccc(-c2ccc(-c3nc(-c4ccc5ccccc5c4)nc(-c4cccc5oc6cccc(-c7ccc(-c8ccccc8)cc7)c6c45)n3)cc2)cc1]

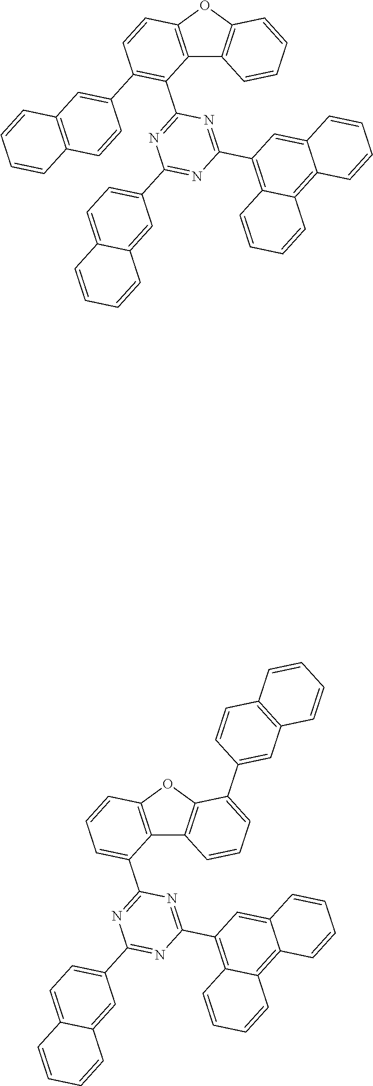 [SMILES: c1ccc2cc(-c3nc(-c4cc5ccccc5c5ccccc45)nc(-c4c(-c5ccc6ccccc6c5)ccc5oc6ccccc6c45)n3)ccc2c1 ; c1ccc2cc(-c3nc(-c4cc5ccccc5c5ccccc45)nc(-c4cccc5oc6c(-c7ccc8ccccc8c7)cccc6c45)n3)ccc2c1]

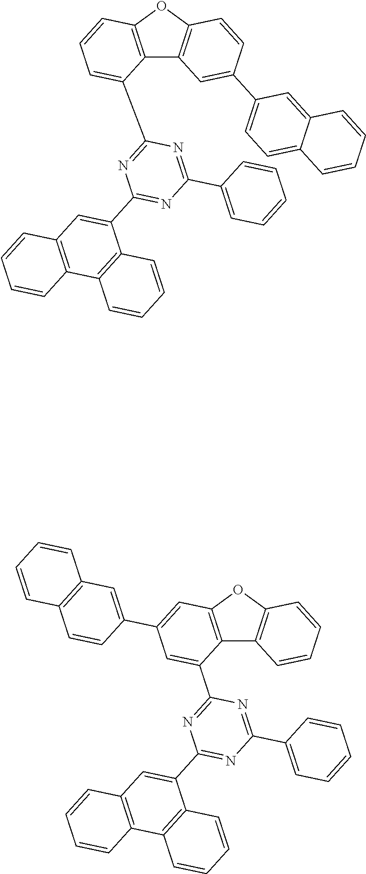 [SMILES: c1ccc(-c2nc(-c3cc4ccccc4c4ccccc34)nc(-c3cccc4oc5ccc(-c6ccc7ccccc7c6)cc5c34)n2)cc1 ; c1ccc(-c2nc(-c3cc4ccccc4c4ccccc34)nc(-c3cc(-c4ccc5ccccc5c4)cc4oc5ccccc5c34)n2)cc1]

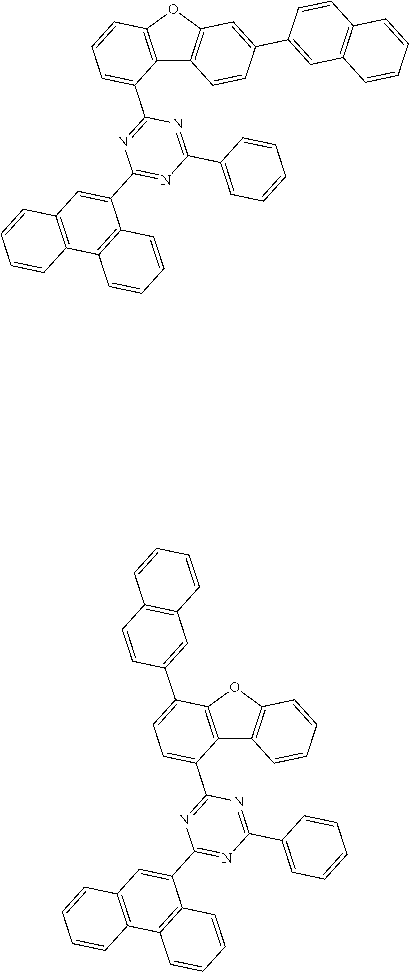 [SMILES: c1ccc(-c2nc(-c3cc4ccccc4c4ccccc34)nc(-c3cccc4oc5cc(-c6ccc7ccccc7c6)ccc5c34)n2)cc1 ; c1ccc(-c2nc(-c3cc4ccccc4c4ccccc34)nc(-c3ccc(-c4ccc5ccccc5c4)c4oc5ccccc5c34)n2)cc1]

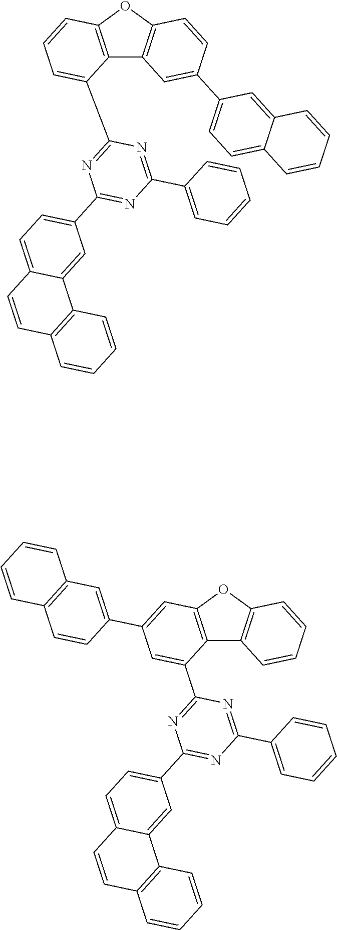 [SMILES: c1ccc(-c2nc(-c3ccc4ccc5ccccc5c4c3)nc(-c3cccc4oc5ccc(-c6ccc7ccccc7c6)cc5c34)n2)cc1 ; c1ccc(-c2nc(-c3ccc4ccc5ccccc5c4c3)nc(-c3cc(-c4ccc5ccccc5c4)cc4oc5ccccc5c34)n2)cc1]

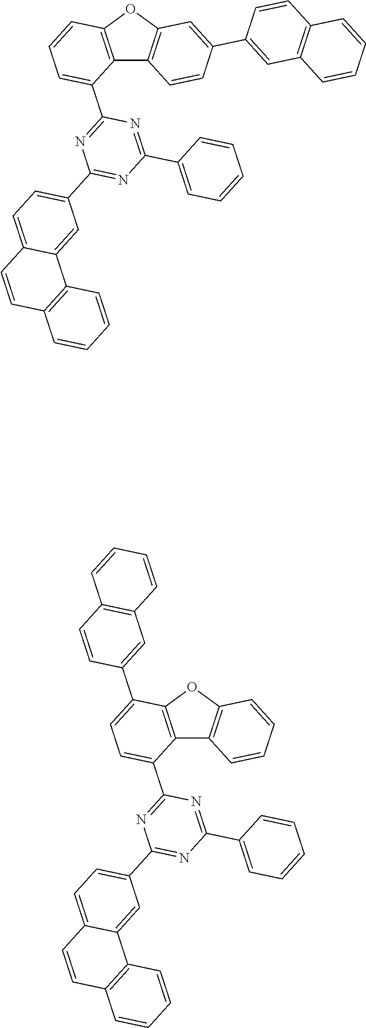 [SMILES: c1ccc(-c2nc(-c3ccc4ccc5ccccc5c4c3)nc(-c3cccc4oc5cc(-c6ccc7ccccc7c6)ccc5c34)n2)cc1 ; c1ccc(-c2nc(-c3ccc4ccc5ccccc5c4c3)nc(-c3ccc(-c4ccc5ccccc5c4)c4oc5ccccc5c34)n2)cc1]

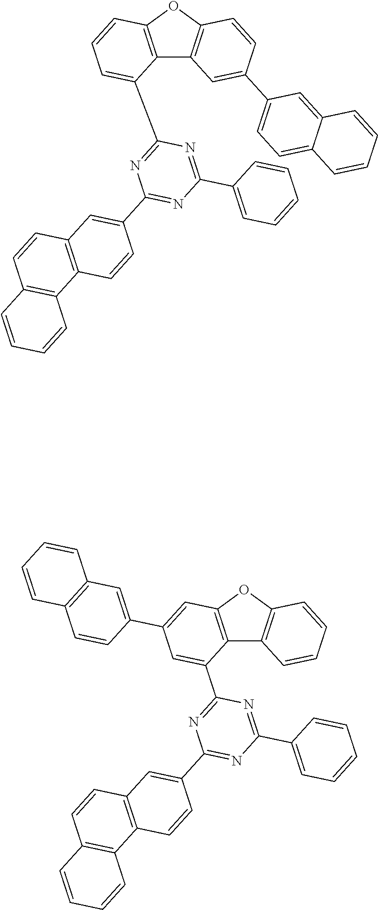 [SMILES: c1ccc(-c2nc(-c3ccc4c(ccc5ccccc54)c3)nc(-c3cccc4oc5ccc(-c6ccc7ccccc7c6)cc5c34)n2)cc1 ; c1ccc(-c2nc(-c3ccc4c(ccc5ccccc54)c3)nc(-c3cc(-c4ccc5ccccc5c4)cc4oc5ccccc5c34)n2)cc1]

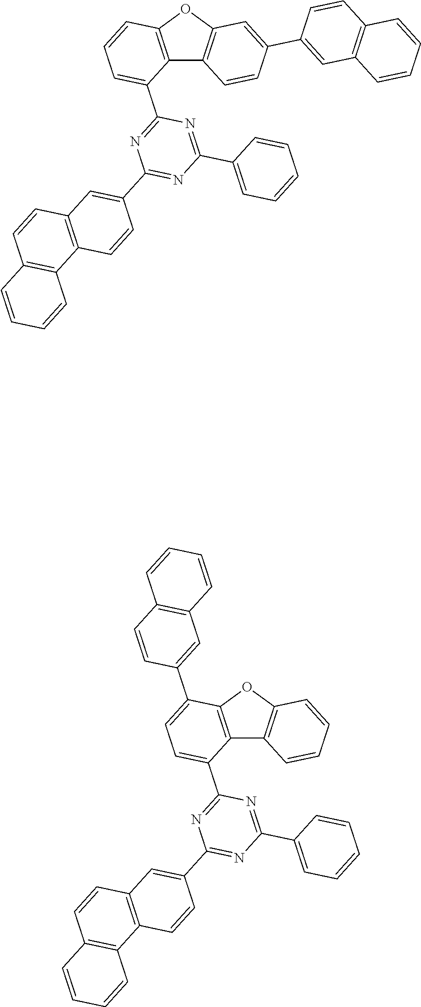 [SMILES: c1ccc(-c2nc(-c3ccc4c(ccc5ccccc54)c3)nc(-c3cccc4oc5cc(-c6ccc7ccccc7c6)ccc5c34)n2)cc1 ; c1ccc(-c2nc(-c3ccc4c(ccc5ccccc54)c3)nc(-c3ccc(-c4ccc5ccccc5c4)c4oc5ccccc5c34)n2)cc1]

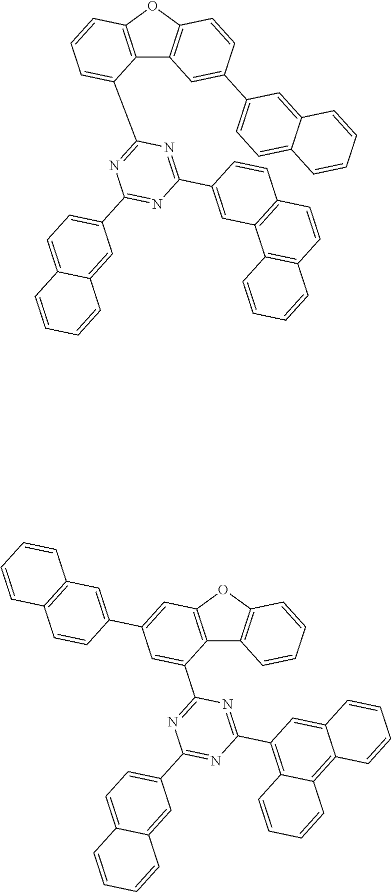 [SMILES: c1ccc2cc(-c3ccc4oc5cccc(-c6nc(-c7ccc8ccccc8c7)nc(-c7ccc8ccc9ccccc9c8c7)n6)c5c4c3)ccc2c1 ; c1ccc2cc(-c3cc(-c4nc(-c5ccc6ccccc6c5)nc(-c5cc6ccccc6c6ccccc56)n4)c4c(c3)oc3ccccc34)ccc2c1]

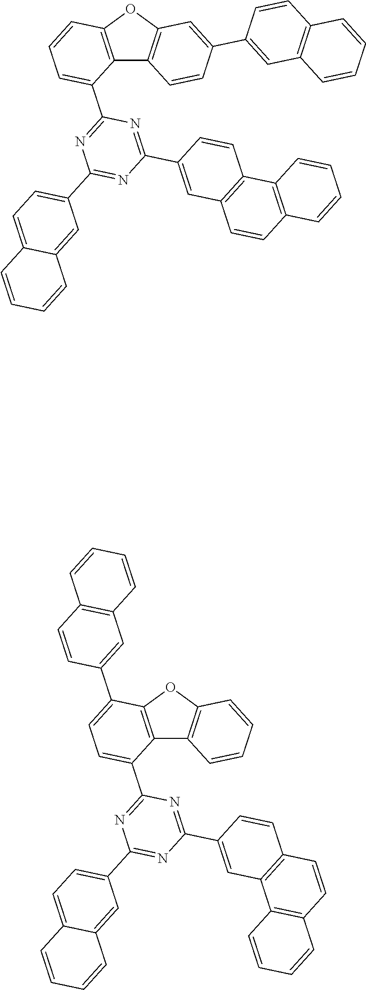 [SMILES: c1ccc2cc(-c3ccc4c(c3)oc3cccc(-c5nc(-c6ccc7ccccc7c6)nc(-c6ccc7c(ccc8ccccc87)c6)n5)c34)ccc2c1 ; c1ccc2cc(-c3nc(-c4ccc5ccc6ccccc6c5c4)nc(-c4ccc(-c5ccc6ccccc6c5)c5oc6ccccc6c45)n3)ccc2c1]

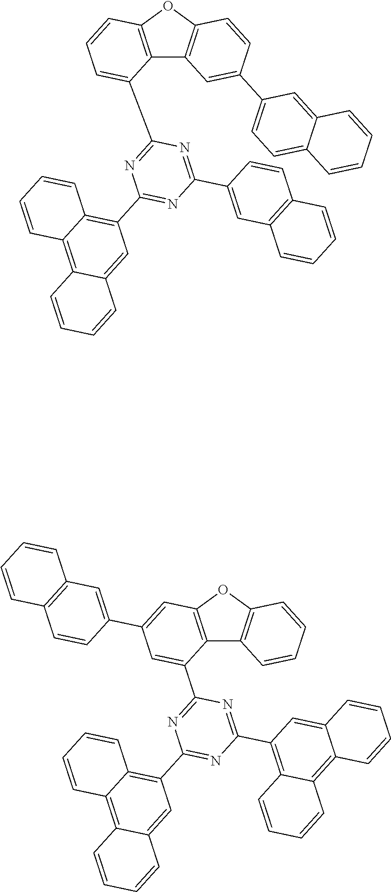 [SMILES: c1ccc2cc(-c3ccc4oc5cccc(-c6nc(-c7ccc8ccccc8c7)nc(-c7cc8ccccc8c8ccccc78)n6)c5c4c3)ccc2c1 ; c1ccc2cc(-c3cc(-c4nc(-c5cc6ccccc6c6ccccc56)nc(-c5cc6ccccc6c6ccccc56)n4)c4c(c3)oc3ccccc34)ccc2c1]

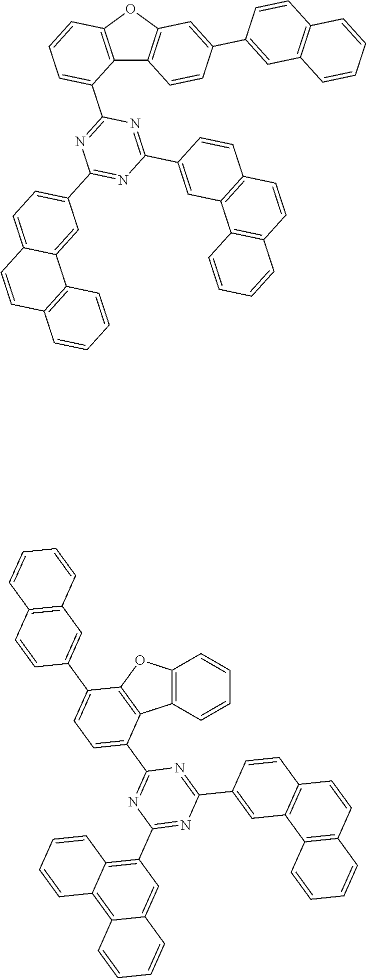 [SMILES: c1ccc2cc(-c3ccc4c(c3)oc3cccc(-c5nc(-c6ccc7ccc8ccccc8c7c6)nc(-c6ccc7ccc8ccccc8c7c6)n5)c34)ccc2c1 ; c1ccc2cc(-c3ccc(-c4nc(-c5ccc6ccc7ccccc7c6c5)nc(-c5cc6ccccc6c6ccccc56)n4)c4c3oc3ccccc34)ccc2c1]

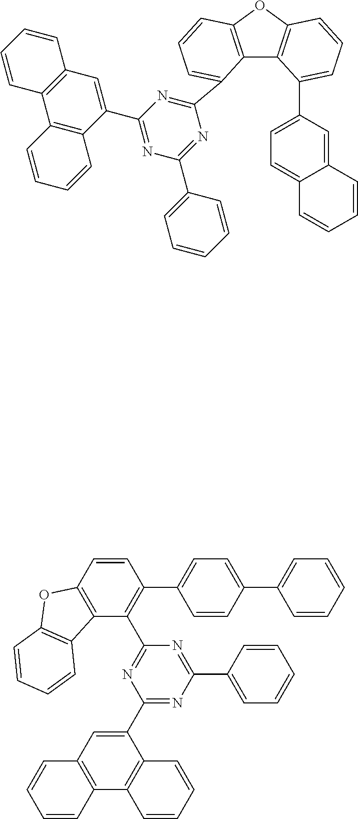 [SMILES: c1ccc(-c2nc(-c3cc4ccccc4c4ccccc34)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccccc4)nc(-c4cc5ccccc5c5ccccc45)n3)cc2)cc1]

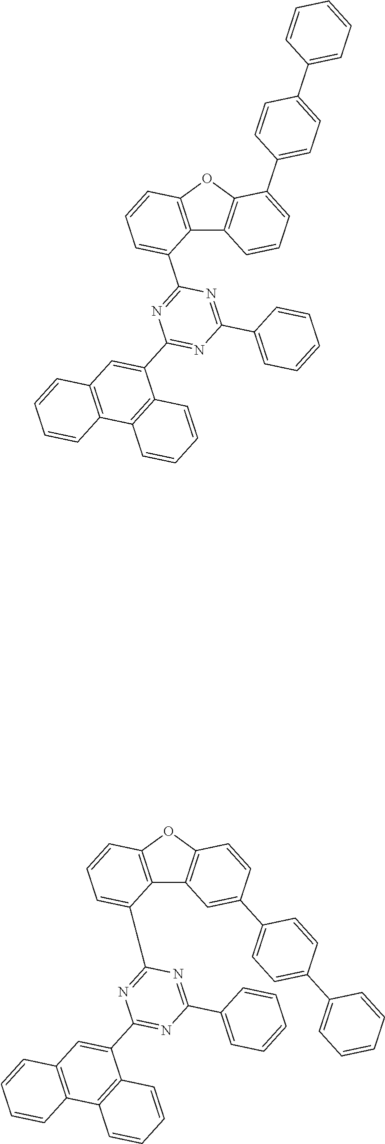 [SMILES: c1ccc(-c2ccc(-c3cccc4c3oc3cccc(-c5nc(-c6ccccc6)nc(-c6cc7ccccc7c7ccccc67)n5)c34)cc2)cc1 ; c1ccc(-c2ccc(-c3ccc4oc5cccc(-c6nc(-c7ccccc7)nc(-c7cc8ccccc8c8ccccc78)n6)c5c4c3)cc2)cc1]

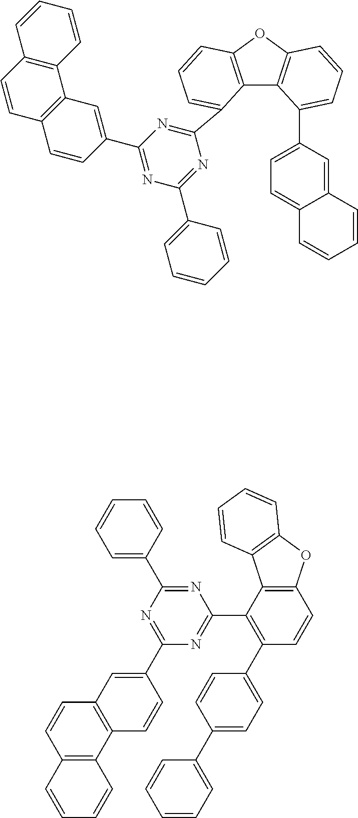 [SMILES: c1ccc(-c2nc(-c3ccc4ccc5ccccc5c4c3)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccccc4)nc(-c4ccc5c(ccc6ccccc65)c4)n3)cc2)cc1]

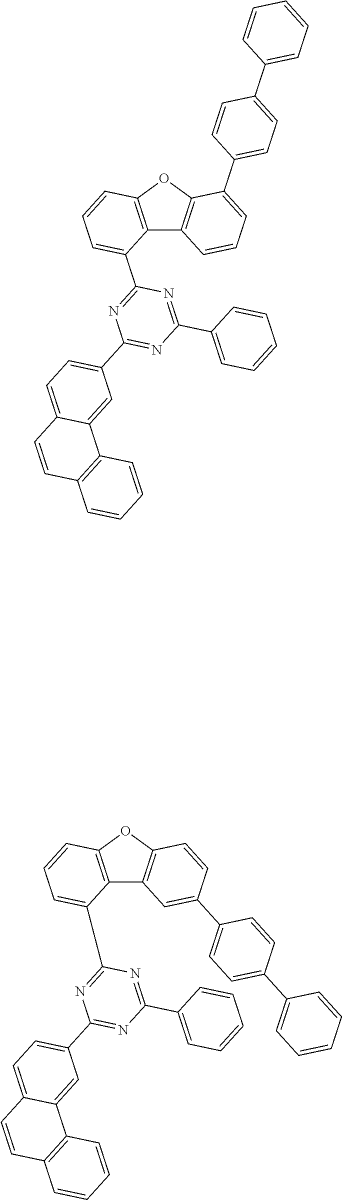 [SMILES: c1ccc(-c2ccc(-c3cccc4c3oc3cccc(-c5nc(-c6ccccc6)nc(-c6ccc7ccc8ccccc8c7c6)n5)c34)cc2)cc1 ; c1ccc(-c2ccc(-c3ccc4oc5cccc(-c6nc(-c7ccccc7)nc(-c7ccc8ccc9ccccc9c8c7)n6)c5c4c3)cc2)cc1]

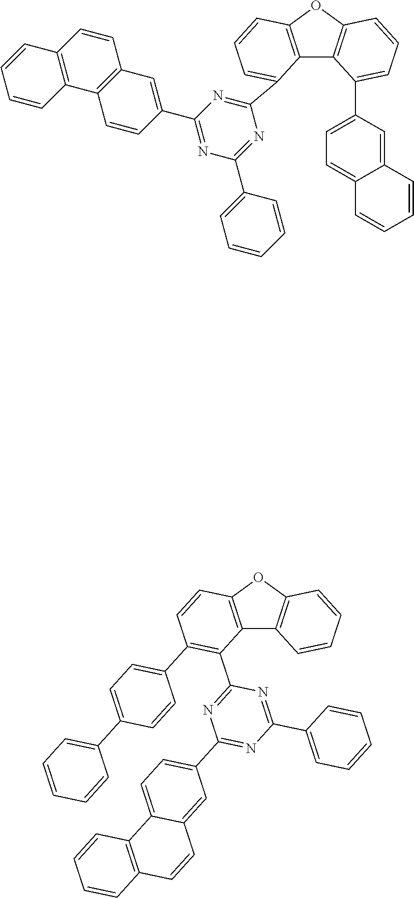 [SMILES: c1ccc(-c2nc(-c3ccc4c(ccc5ccccc54)c3)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccccc4)nc(-c4ccc5c(ccc6ccccc65)c4)n3)cc2)cc1]

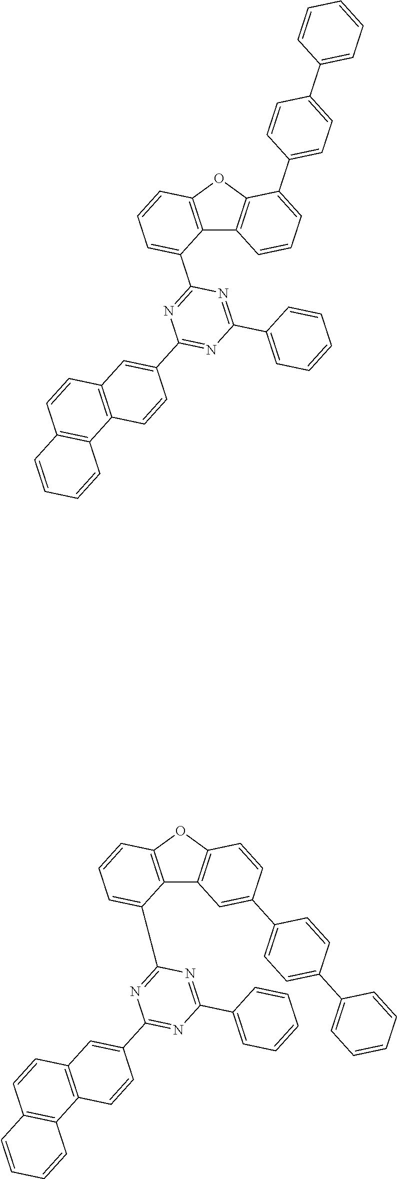 [SMILES: c1ccc(-c2ccc(-c3cccc4c3oc3cccc(-c5nc(-c6ccccc6)nc(-c6ccc7c(ccc8ccccc87)c6)n5)c34)cc2)cc1 ; c1ccc(-c2ccc(-c3ccc4oc5cccc(-c6nc(-c7ccccc7)nc(-c7ccc8c(ccc9ccccc98)c7)n6)c5c4c3)cc2)cc1]

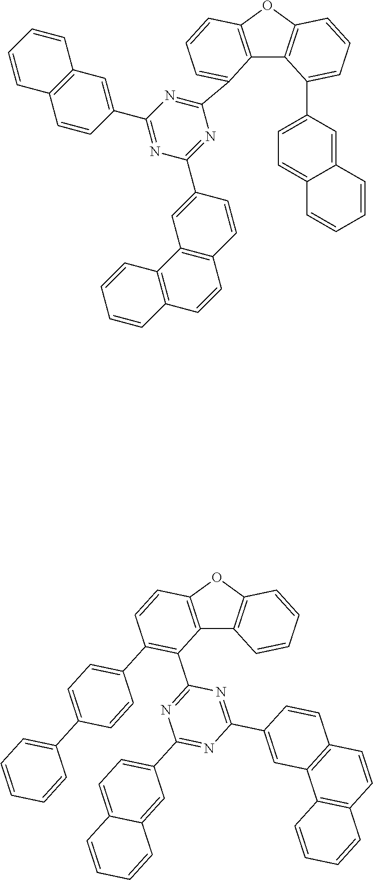 [SMILES: c1ccc2cc(-c3nc(-c4ccc5ccc6ccccc6c5c4)nc(-c4cccc5oc6cccc(-c7ccc8ccccc8c7)c6c45)n3)ccc2c1 ; c1ccc(-c2ccc(-c3ccc4oc5ccccc5c4c3-c3nc(-c4ccc5ccccc5c4)nc(-c4ccc5ccc6ccccc6c5c4)n3)cc2)cc1]

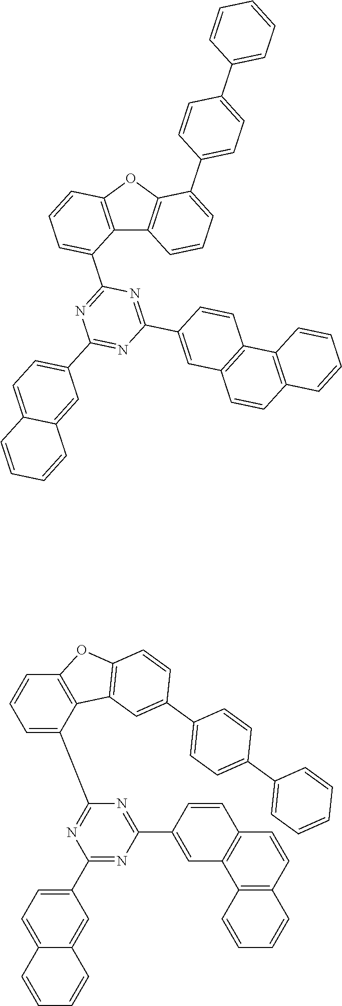 [SMILES: c1ccc(-c2ccc(-c3cccc4c3oc3cccc(-c5nc(-c6ccc7ccccc7c6)nc(-c6ccc7c(ccc8ccccc87)c6)n5)c34)cc2)cc1 ; c1ccc(-c2ccc(-c3ccc4oc5cccc(-c6nc(-c7ccc8ccccc8c7)nc(-c7ccc8ccc9ccccc9c8c7)n6)c5c4c3)cc2)cc1]

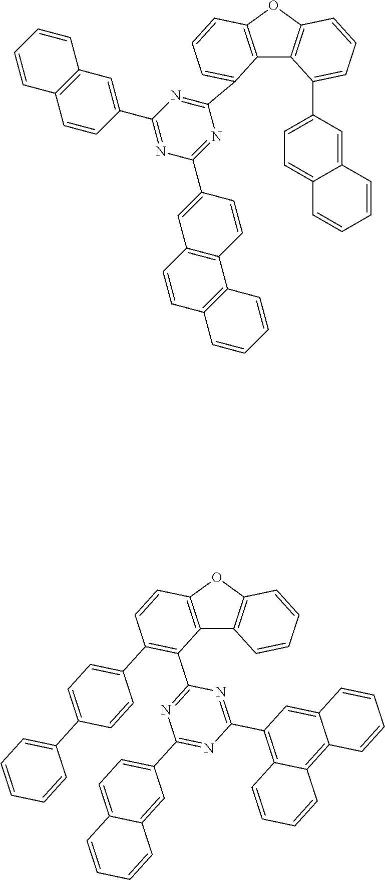 [SMILES: c1ccc2cc(-c3nc(-c4ccc5c(ccc6ccccc65)c4)nc(-c4cccc5oc6cccc(-c7ccc8ccccc8c7)c6c45)n3)ccc2c1 ; c1ccc(-c2ccc(-c3ccc4oc5ccccc5c4c3-c3nc(-c4ccc5ccccc5c4)nc(-c4cc5ccccc5c5ccccc45)n3)cc2)cc1]

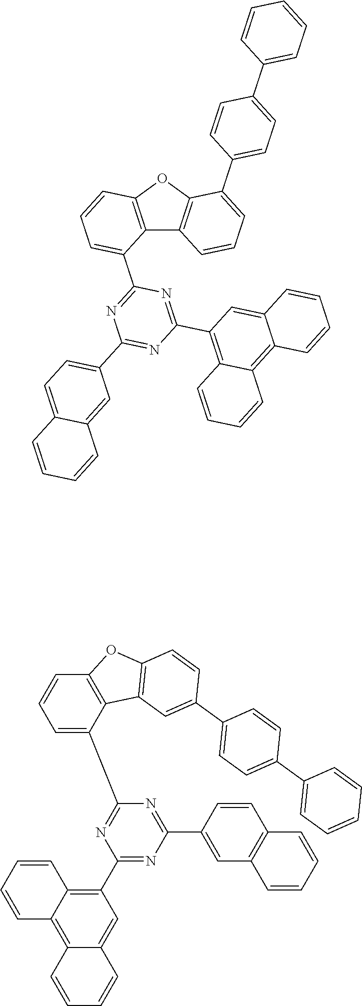 [SMILES: c1ccc(-c2ccc(-c3cccc4c3oc3cccc(-c5nc(-c6ccc7ccccc7c6)nc(-c6cc7ccccc7c7ccccc67)n5)c34)cc2)cc1 ; c1ccc(-c2ccc(-c3ccc4oc5cccc(-c6nc(-c7ccc8ccccc8c7)nc(-c7cc8ccccc8c8ccccc78)n6)c5c4c3)cc2)cc1]

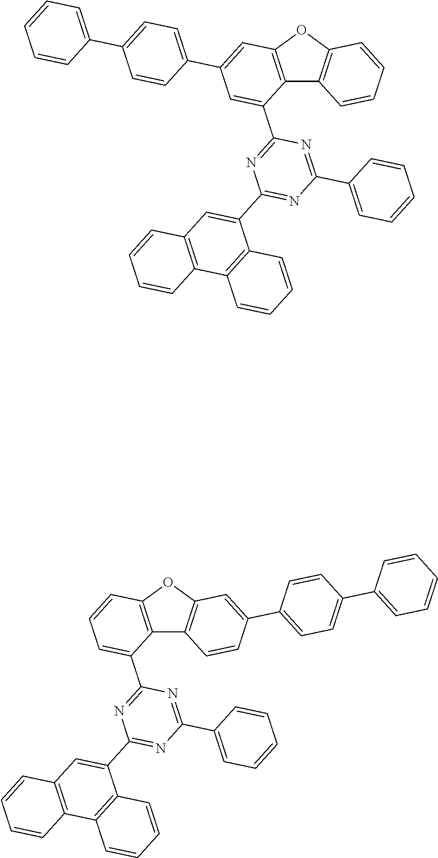 [SMILES: c1ccc(-c2ccc(-c3cc(-c4nc(-c5ccccc5)nc(-c5cc6ccccc6c6ccccc56)n4)c4c(c3)oc3ccccc34)cc2)cc1 ; c1ccc(-c2ccc(-c3ccc4c(c3)oc3cccc(-c5nc(-c6ccccc6)nc(-c6cc7ccccc7c7ccccc67)n5)c34)cc2)cc1]

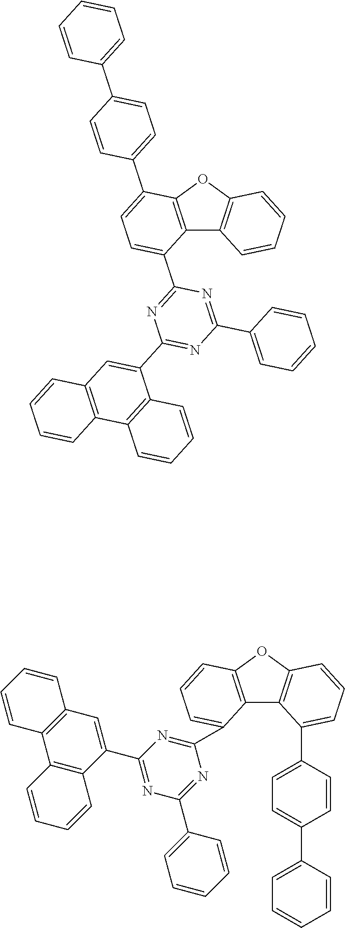 [SMILES: c1ccc(-c2ccc(-c3ccc(-c4nc(-c5ccccc5)nc(-c5cc6ccccc6c6ccccc56)n4)c4c3oc3ccccc34)cc2)cc1 ; c1ccc(-c2ccc(-c3cccc4oc5cccc(-c6nc(-c7ccccc7)nc(-c7cc8ccccc8c8ccccc78)n6)c5c34)cc2)cc1]

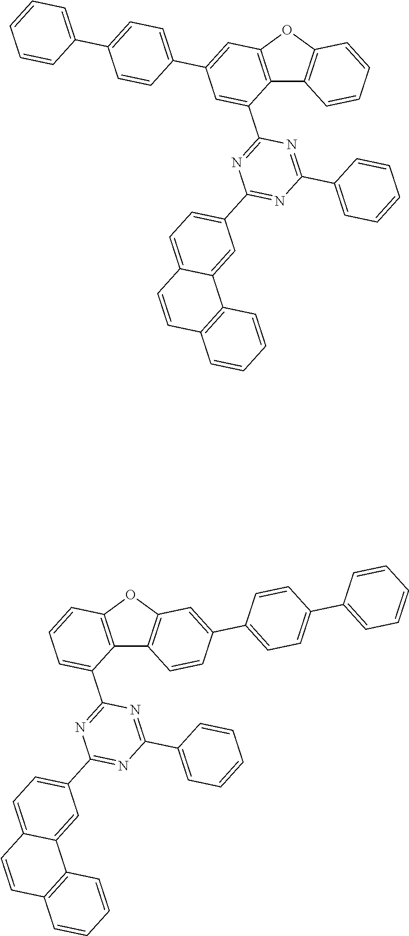 [SMILES: c1ccc(-c2ccc(-c3cc(-c4nc(-c5ccccc5)nc(-c5ccc6ccc7ccccc7c6c5)n4)c4c(c3)oc3ccccc34)cc2)cc1 ; c1ccc(-c2ccc(-c3ccc4c(c3)oc3cccc(-c5nc(-c6ccccc6)nc(-c6ccc7ccc8ccccc8c7c6)n5)c34)cc2)cc1]

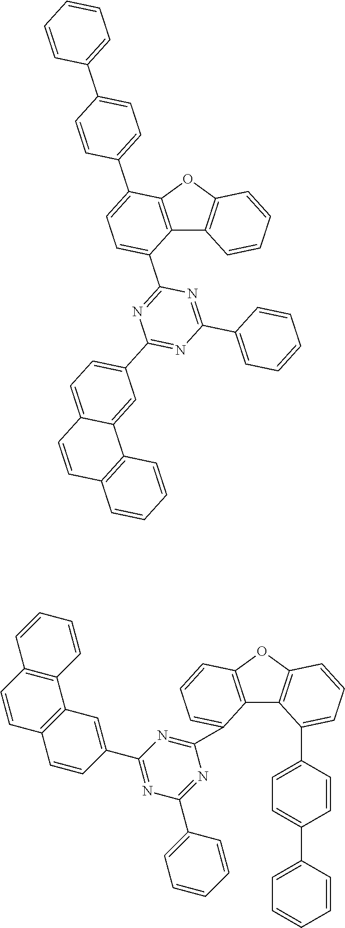 [SMILES: c1ccc(-c2ccc(-c3ccc(-c4nc(-c5ccccc5)nc(-c5ccc6ccc7ccccc7c6c5)n4)c4c3oc3ccccc34)cc2)cc1 ; c1ccc(-c2ccc(-c3cccc4oc5cccc(-c6nc(-c7ccccc7)nc(-c7ccc8ccc9ccccc9c8c7)n6)c5c34)cc2)cc1]

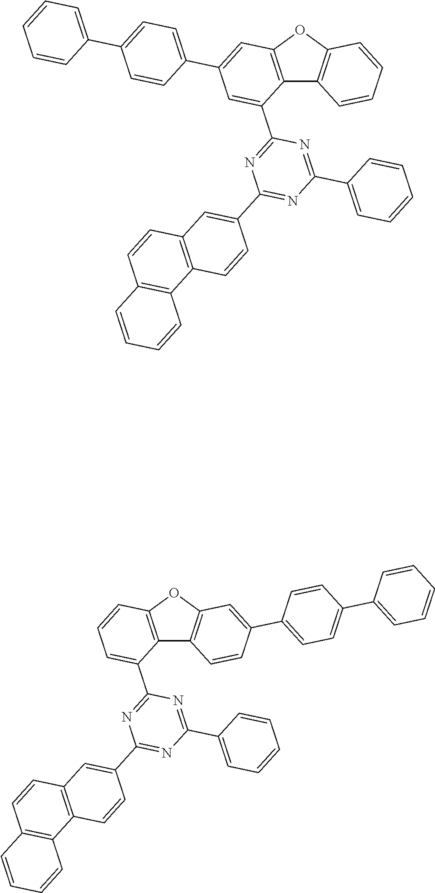 [SMILES: c1ccc(-c2ccc(-c3cc(-c4nc(-c5ccccc5)nc(-c5ccc6c(ccc7ccccc76)c5)n4)c4c(c3)oc3ccccc34)cc2)cc1 ; c1ccc(-c2ccc(-c3ccc4c(c3)oc3cccc(-c5nc(-c6ccccc6)nc(-c6ccc7c(ccc8ccccc87)c6)n5)c34)cc2)cc1]

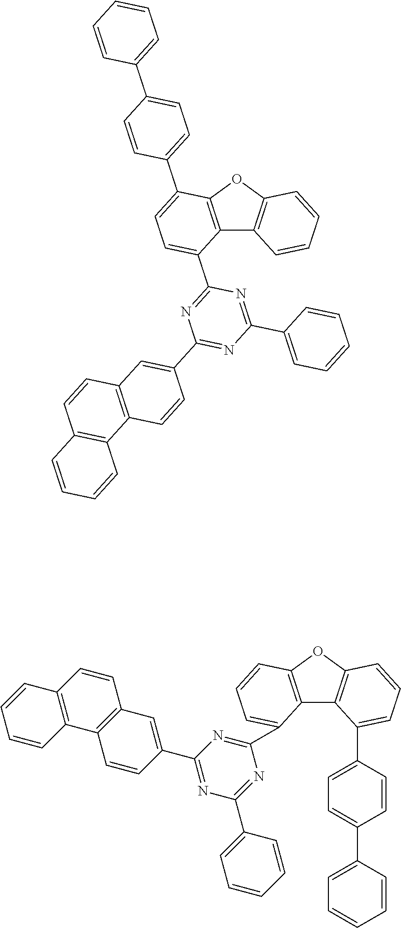 [SMILES: c1ccc(-c2ccc(-c3ccc(-c4nc(-c5ccccc5)nc(-c5ccc6c(ccc7ccccc76)c5)n4)c4c3oc3ccccc34)cc2)cc1 ; c1ccc(-c2ccc(-c3cccc4oc5cccc(-c6nc(-c7ccccc7)nc(-c7ccc8c(ccc9ccccc98)c7)n6)c5c34)cc2)cc1]

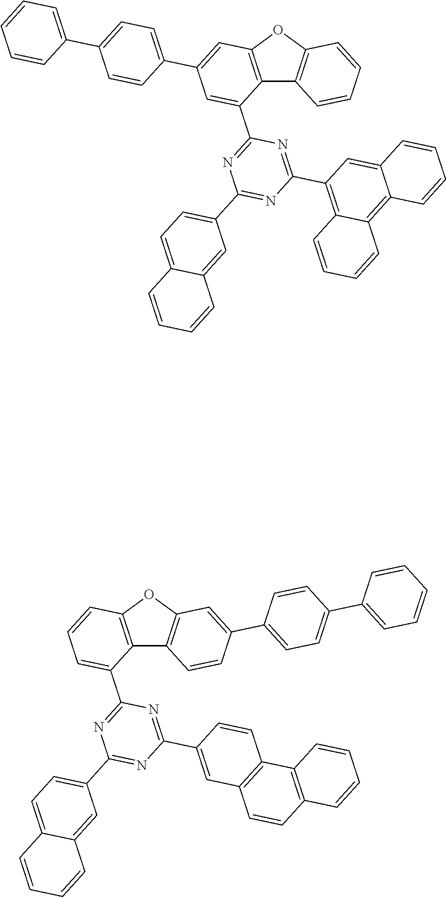 [SMILES: c1ccc(-c2ccc(-c3cc(-c4nc(-c5ccc6ccccc6c5)nc(-c5cc6ccccc6c6ccccc56)n4)c4c(c3)oc3ccccc34)cc2)cc1 ; c1ccc(-c2ccc(-c3ccc4c(c3)oc3cccc(-c5nc(-c6ccc7ccccc7c6)nc(-c6ccc7c(ccc8ccccc87)c6)n5)c34)cc2)cc1]

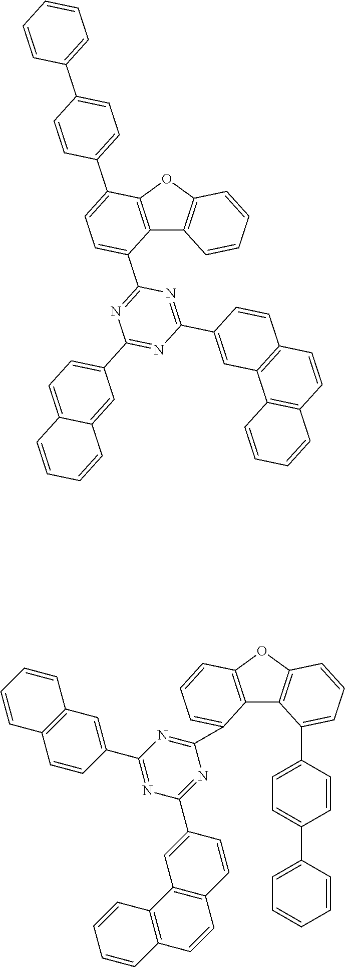 [SMILES: c1ccc(-c2ccc(-c3ccc(-c4nc(-c5ccc6ccccc6c5)nc(-c5ccc6ccc7ccccc7c6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccc9ccccc9c8c7)n6)c5c34)cc2)cc1]

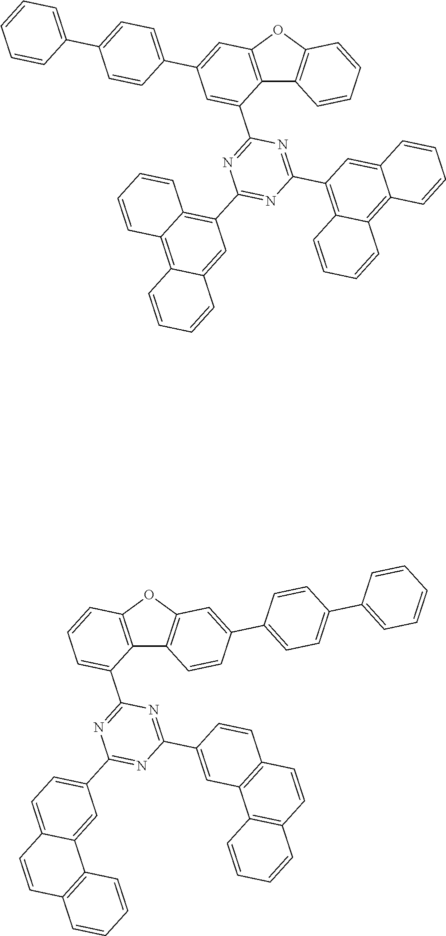 [SMILES: c1ccc(-c2ccc(-c3cc(-c4nc(-c5cc6ccccc6c6ccccc56)nc(-c5cc6ccccc6c6ccccc56)n4)c4c(c3)oc3ccccc34)cc2)cc1 ; c1ccc(-c2ccc(-c3ccc4c(c3)oc3cccc(-c5nc(-c6ccc7ccc8ccccc8c7c6)nc(-c6ccc7ccc8ccccc8c7c6)n5)c34)cc2)cc1]

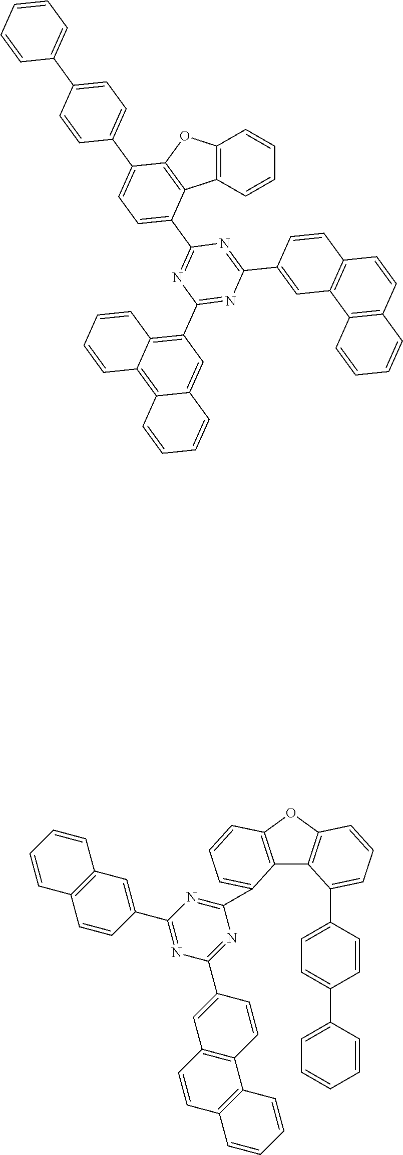 [SMILES: c1ccc(-c2ccc(-c3ccc(-c4nc(-c5ccc6ccc7ccccc7c6c5)nc(-c5cc6ccccc6c6ccccc56)n4)c4c3oc3ccccc34)cc2)cc1 ; c1ccc(-c2ccc(-c3cccc4oc5cccc(-c6nc(-c7ccc8ccccc8c7)nc(-c7ccc8c(ccc9ccccc98)c7)n6)c5c34)cc2)cc1]

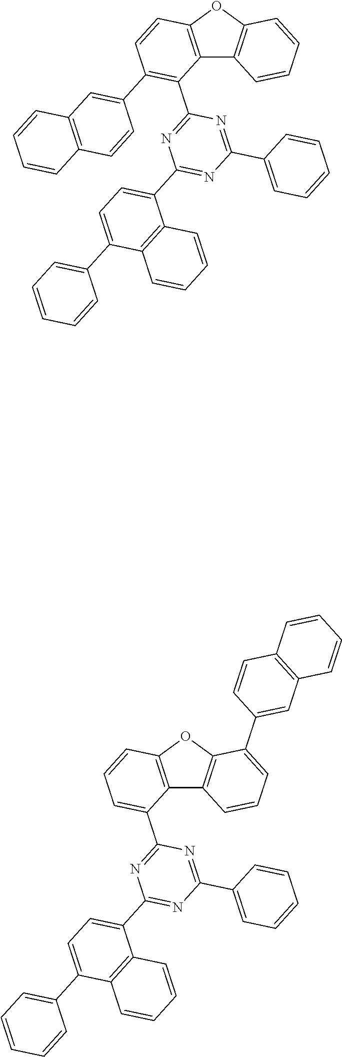 [SMILES: c1ccc(-c2nc(-c3ccc(-c4ccccc4)c4ccccc34)nc(-c3c(-c4ccc5ccccc5c4)ccc4oc5ccccc5c34)n2)cc1 ; c1ccc(-c2nc(-c3ccc(-c4ccccc4)c4ccccc34)nc(-c3cccc4oc5c(-c6ccc7ccccc7c6)cccc5c34)n2)cc1]

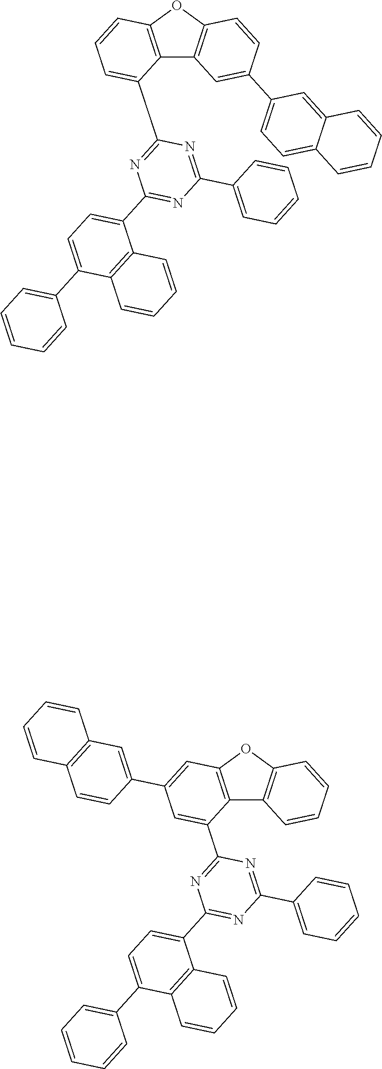 [SMILES: c1ccc(-c2nc(-c3ccc(-c4ccccc4)c4ccccc34)nc(-c3cccc4oc5ccc(-c6ccc7ccccc7c6)cc5c34)n2)cc1 ; c1ccc(-c2nc(-c3ccc(-c4ccccc4)c4ccccc34)nc(-c3cc(-c4ccc5ccccc5c4)cc4oc5ccccc5c34)n2)cc1]

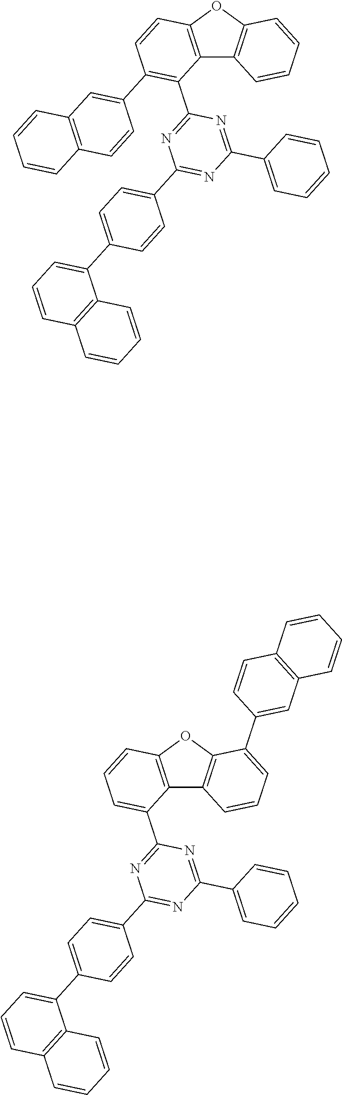 [SMILES: c1ccc(-c2nc(-c3ccc(-c4cccc5ccccc45)cc3)nc(-c3c(-c4ccc5ccccc5c4)ccc4oc5ccccc5c34)n2)cc1 ; c1ccc(-c2nc(-c3ccc(-c4cccc5ccccc45)cc3)nc(-c3cccc4oc5c(-c6ccc7ccccc7c6)cccc5c34)n2)cc1]

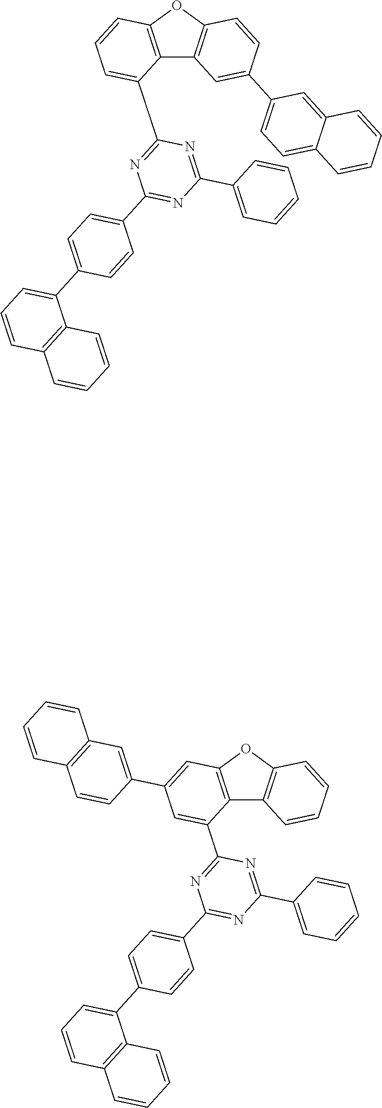 [SMILES: c1ccc(-c2nc(-c3ccc(-c4cccc5ccccc45)cc3)nc(-c3cccc4oc5ccc(-c6ccc7ccccc7c6)cc5c34)n2)cc1 ; c1ccc(-c2nc(-c3ccc(-c4cccc5ccccc45)cc3)nc(-c3cc(-c4ccc5ccccc5c4)cc4oc5ccccc5c34)n2)cc1]

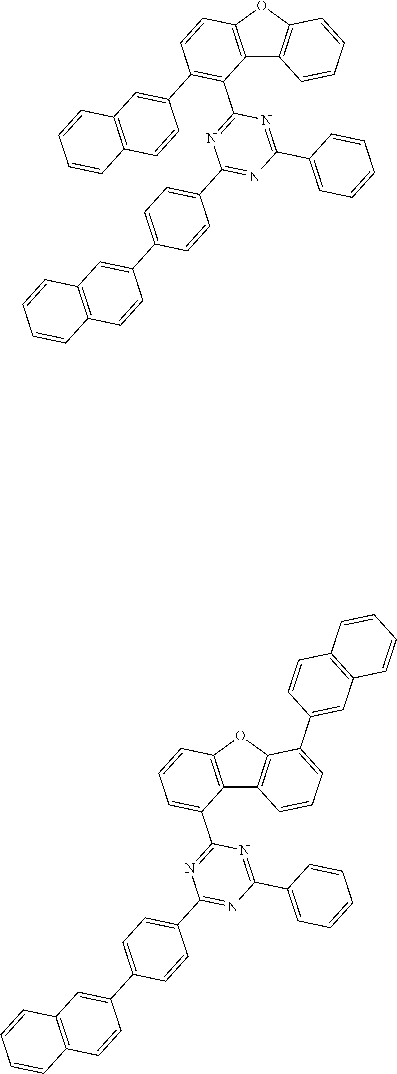 [SMILES: c1ccc(-c2nc(-c3ccc(-c4ccc5ccccc5c4)cc3)nc(-c3c(-c4ccc5ccccc5c4)ccc4oc5ccccc5c34)n2)cc1 ; c1ccc(-c2nc(-c3ccc(-c4ccc5ccccc5c4)cc3)nc(-c3cccc4oc5c(-c6ccc7ccccc7c6)cccc5c34)n2)cc1]

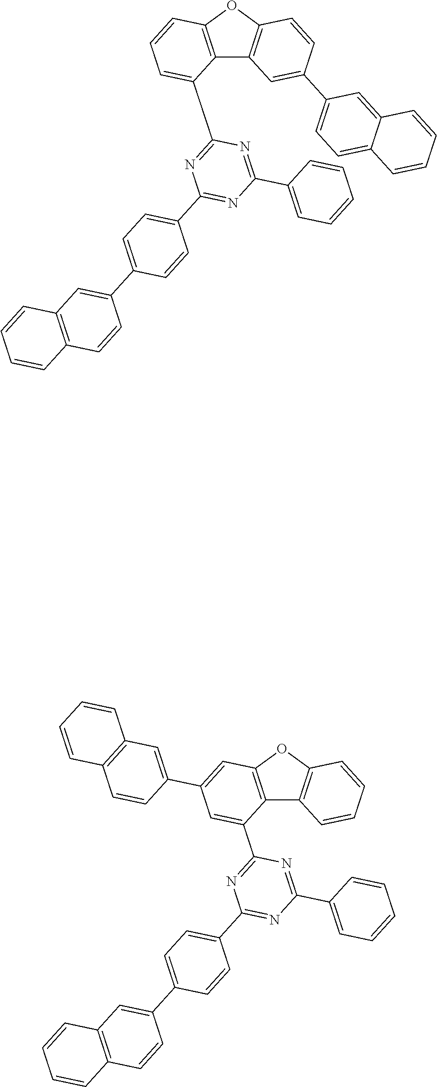 [SMILES: c1ccc(-c2nc(-c3ccc(-c4ccc5ccccc5c4)cc3)nc(-c3cccc4oc5ccc(-c6ccc7ccccc7c6)cc5c34)n2)cc1 ; c1ccc(-c2nc(-c3ccc(-c4ccc5ccccc5c4)cc3)nc(-c3cc(-c4ccc5ccccc5c4)cc4oc5ccccc5c34)n2)cc1]

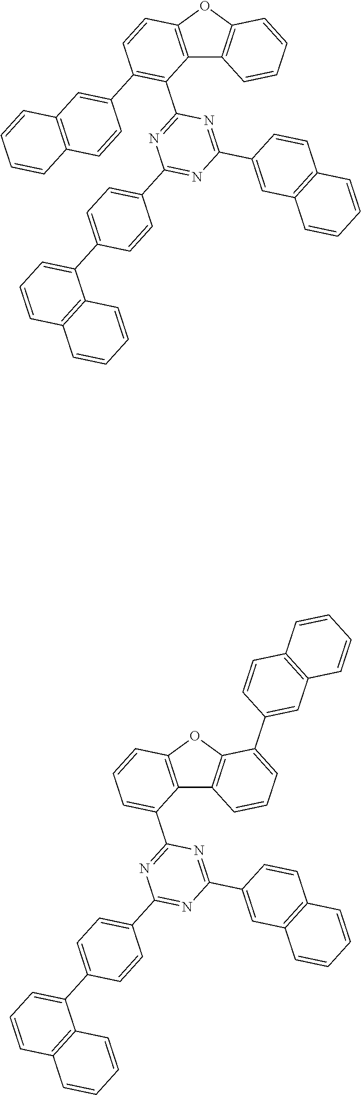 [SMILES: c1ccc2cc(-c3nc(-c4ccc(-c5cccc6ccccc56)cc4)nc(-c4c(-c5ccc6ccccc6c5)ccc5oc6ccccc6c45)n3)ccc2c1 ; c1ccc2cc(-c3nc(-c4ccc(-c5cccc6ccccc56)cc4)nc(-c4cccc5oc6c(-c7ccc8ccccc8c7)cccc6c45)n3)ccc2c1]

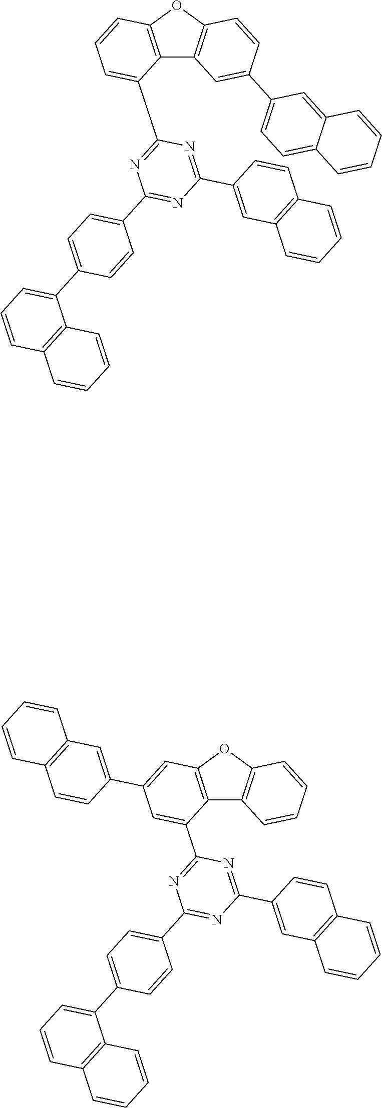 [SMILES: c1ccc2cc(-c3ccc4oc5cccc(-c6nc(-c7ccc(-c8cccc9ccccc89)cc7)nc(-c7ccc8ccccc8c7)n6)c5c4c3)ccc2c1 ; c1ccc2cc(-c3cc(-c4nc(-c5ccc(-c6cccc7ccccc67)cc5)nc(-c5ccc6ccccc6c5)n4)c4c(c3)oc3ccccc34)ccc2c1]

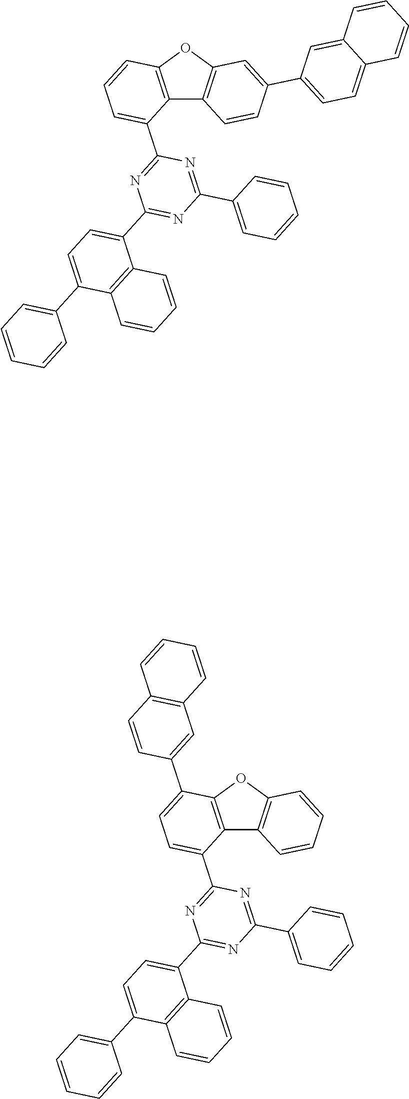 [SMILES: c1ccc(-c2nc(-c3ccc(-c4ccccc4)c4ccccc34)nc(-c3cccc4oc5cc(-c6ccc7ccccc7c6)ccc5c34)n2)cc1 ; c1ccc(-c2nc(-c3ccc(-c4ccccc4)c4ccccc34)nc(-c3ccc(-c4ccc5ccccc5c4)c4oc5ccccc5c34)n2)cc1]

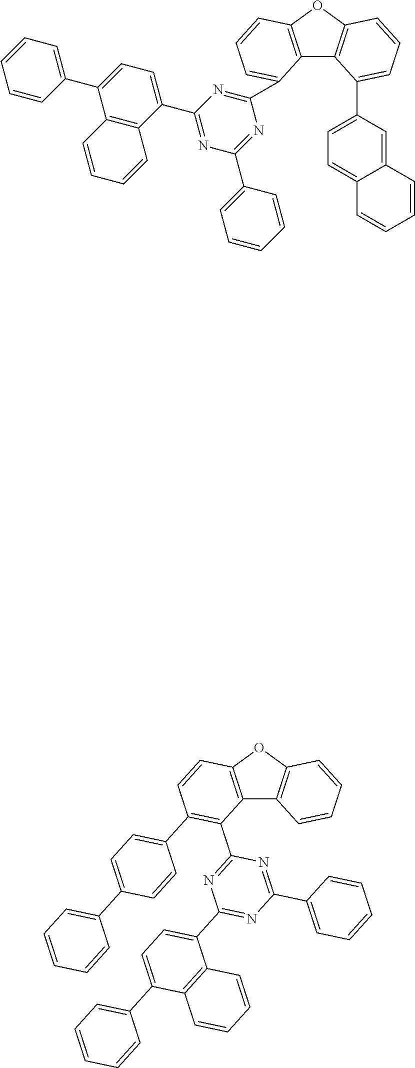 [SMILES: c1ccc(-c2nc(-c3ccc(-c4ccccc4)c4ccccc34)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccccc4)nc(-c4ccc(-c5ccccc5)c5ccccc45)n3)cc2)cc1]

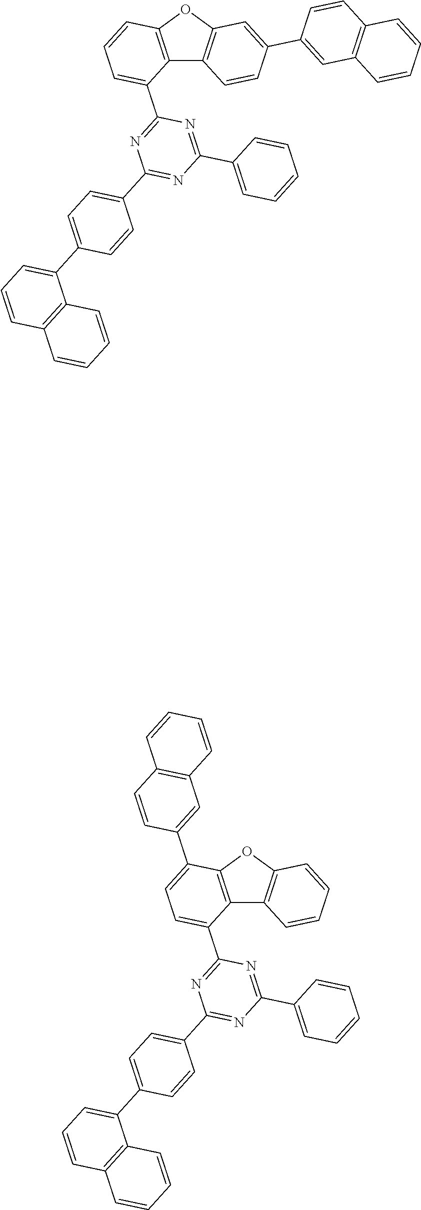 [SMILES: c1ccc(-c2nc(-c3ccc(-c4cccc5ccccc45)cc3)nc(-c3cccc4oc5cc(-c6ccc7ccccc7c6)ccc5c34)n2)cc1 ; c1ccc(-c2nc(-c3ccc(-c4cccc5ccccc45)cc3)nc(-c3ccc(-c4ccc5ccccc5c4)c4oc5ccccc5c34)n2)cc1]

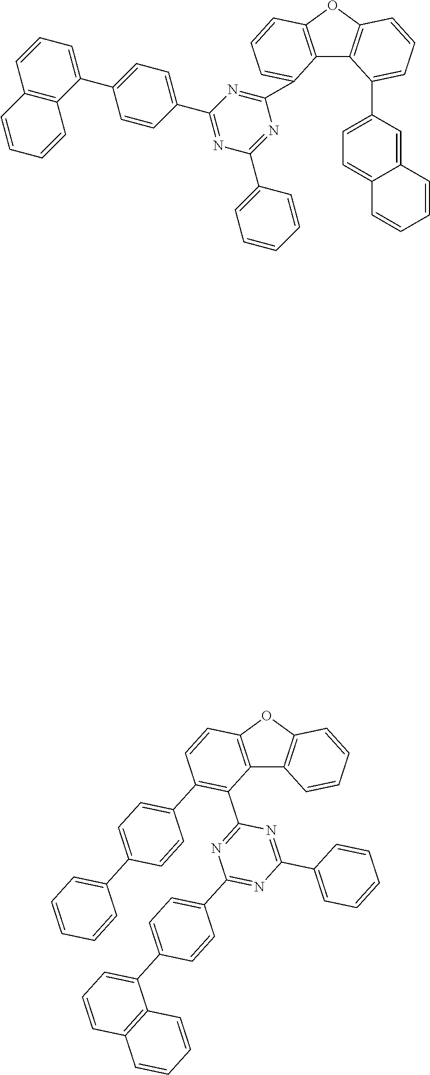 [SMILES: c1ccc(-c2nc(-c3ccc(-c4cccc5ccccc45)cc3)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccccc4)nc(-c4ccc(-c5cccc6ccccc56)cc4)n3)cc2)cc1]

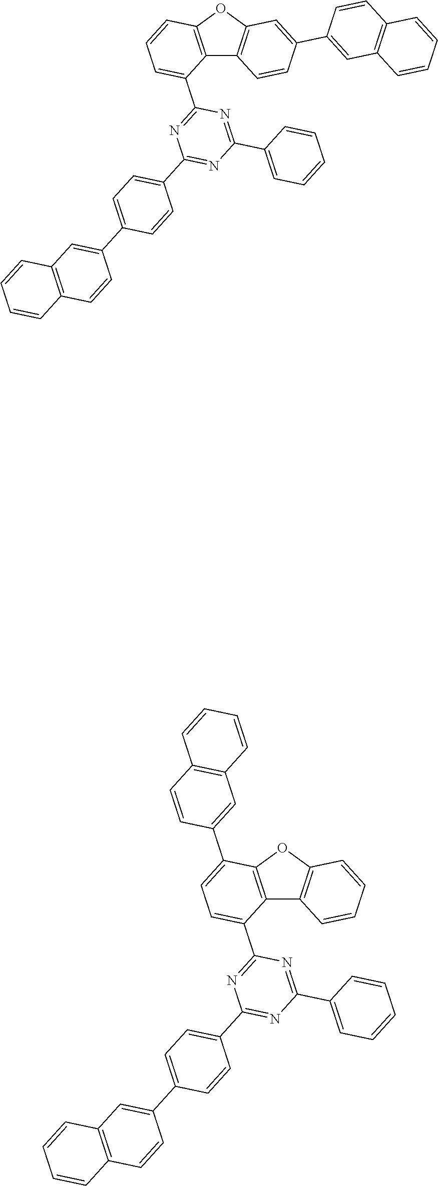 [SMILES: c1ccc(-c2nc(-c3ccc(-c4ccc5ccccc5c4)cc3)nc(-c3cccc4oc5cc(-c6ccc7ccccc7c6)ccc5c34)n2)cc1 ; c1ccc(-c2nc(-c3ccc(-c4ccc5ccccc5c4)cc3)nc(-c3ccc(-c4ccc5ccccc5c4)c4oc5ccccc5c34)n2)cc1]

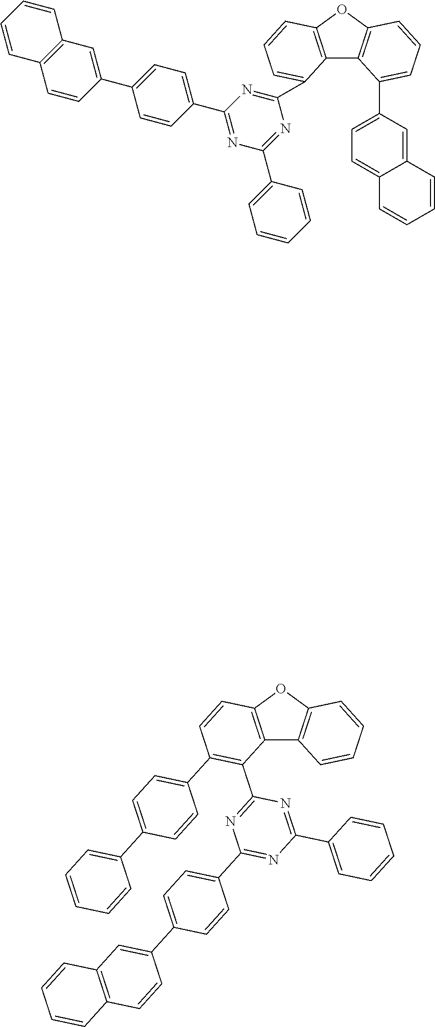 [SMILES: c1ccc(-c2nc(-c3ccc(-c4ccc5ccccc5c4)cc3)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccccc4)nc(-c4ccc(-c5ccc6ccccc6c5)cc4)n3)cc2)cc1]

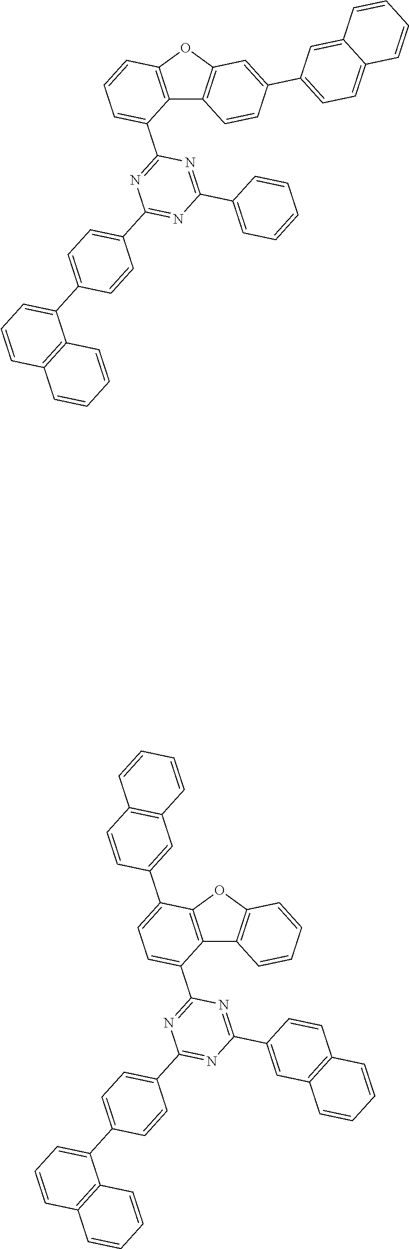 [SMILES: c1ccc(-c2nc(-c3ccc(-c4cccc5ccccc45)cc3)nc(-c3cccc4oc5cc(-c6ccc7ccccc7c6)ccc5c34)n2)cc1 ; c1ccc2cc(-c3nc(-c4ccc(-c5cccc6ccccc56)cc4)nc(-c4ccc(-c5ccc6ccccc6c5)c5oc6ccccc6c45)n3)ccc2c1]

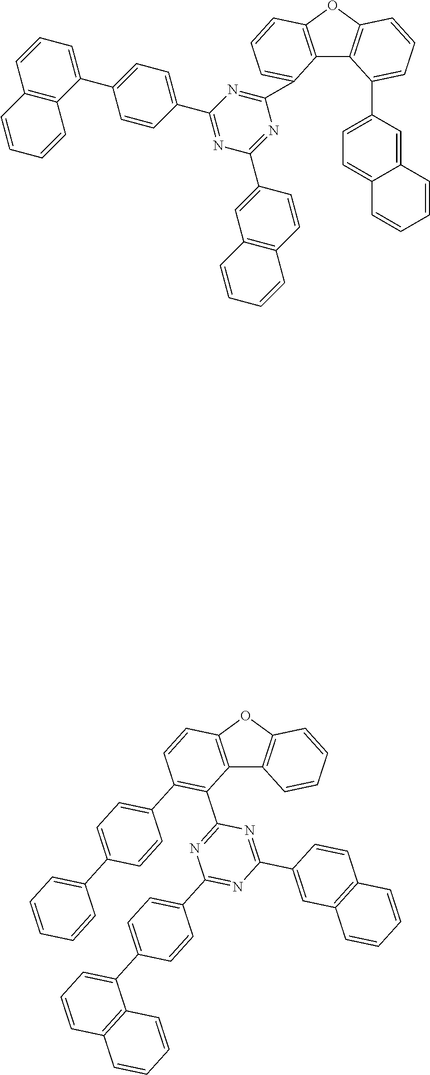 [SMILES: c1ccc2cc(-c3nc(-c4ccc(-c5cccc6ccccc56)cc4)nc(-c4cccc5oc6cccc(-c7ccc8ccccc8c7)c6c45)n3)ccc2c1 ; c1ccc(-c2ccc(-c3ccc4oc5ccccc5c4c3-c3nc(-c4ccc(-c5cccc6ccccc56)cc4)nc(-c4ccc5ccccc5c4)n3)cc2)cc1]

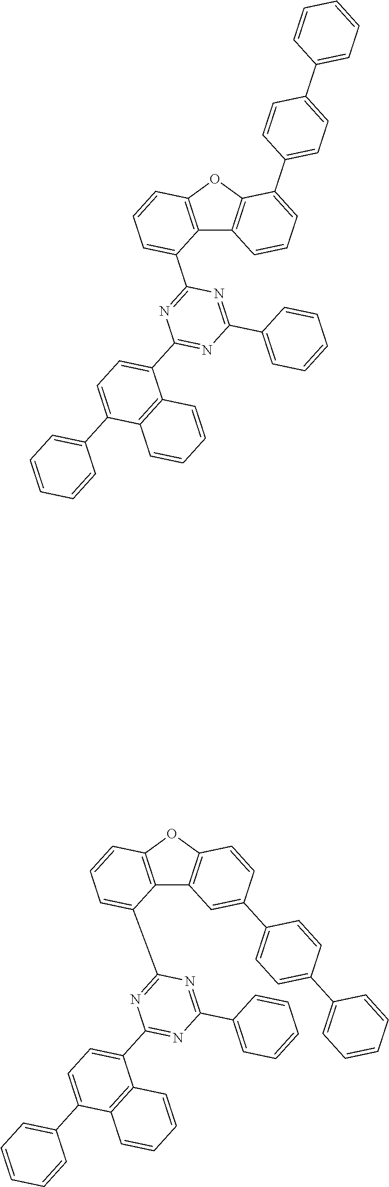 [SMILES: c1ccc(-c2ccc(-c3cccc4c3oc3cccc(-c5nc(-c6ccccc6)nc(-c6ccc(-c7ccccc7)c7ccccc67)n5)c34)cc2)cc1 ; c1ccc(-c2ccc(-c3ccc4oc5cccc(-c6nc(-c7ccccc7)nc(-c7ccc(-c8ccccc8)c8ccccc78)n6)c5c4c3)cc2)cc1]

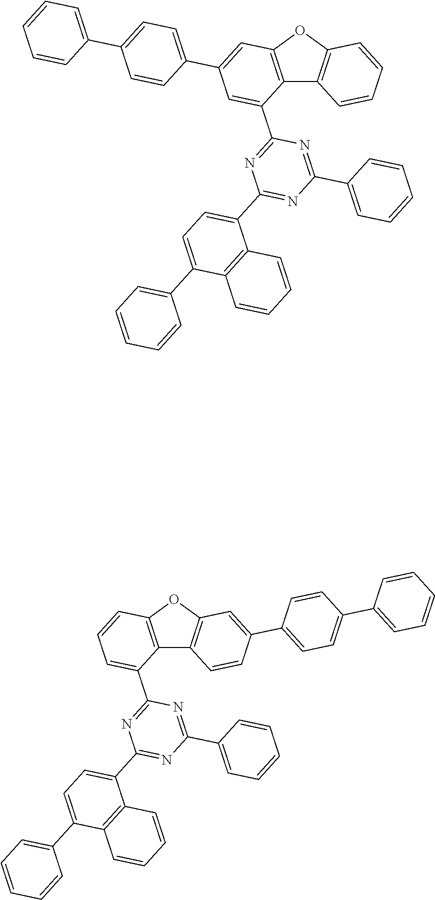 [SMILES: c1ccc(-c2ccc(-c3cc(-c4nc(-c5ccccc5)nc(-c5ccc(-c6ccccc6)c6ccccc56)n4)c4c(c3)oc3ccccc34)cc2)cc1 ; c1ccc(-c2ccc(-c3ccc4c(c3)oc3cccc(-c5nc(-c6ccccc6)nc(-c6ccc(-c7ccccc7)c7ccccc67)n5)c34)cc2)cc1]

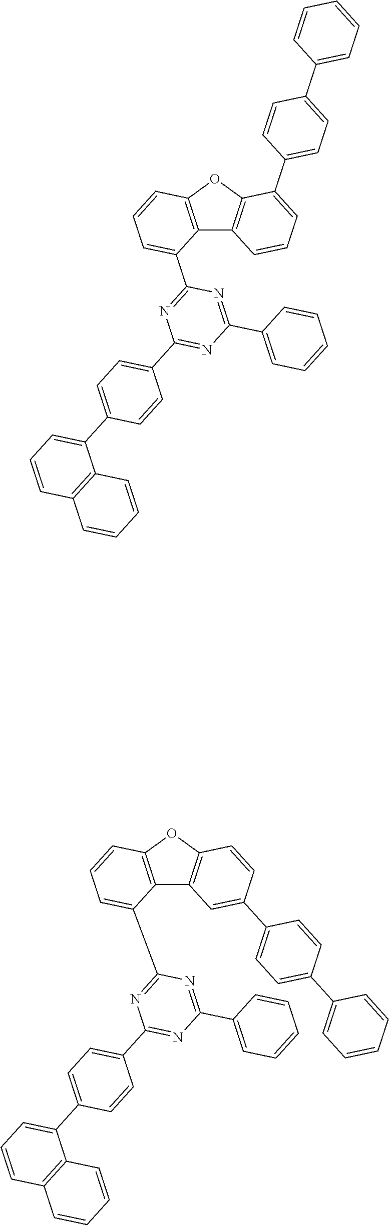 [SMILES: c1ccc(-c2ccc(-c3cccc4c3oc3cccc(-c5nc(-c6ccccc6)nc(-c6ccc(-c7cccc8ccccc78)cc6)n5)c34)cc2)cc1 ; c1ccc(-c2ccc(-c3ccc4oc5cccc(-c6nc(-c7ccccc7)nc(-c7ccc(-c8cccc9ccccc89)cc7)n6)c5c4c3)cc2)cc1]

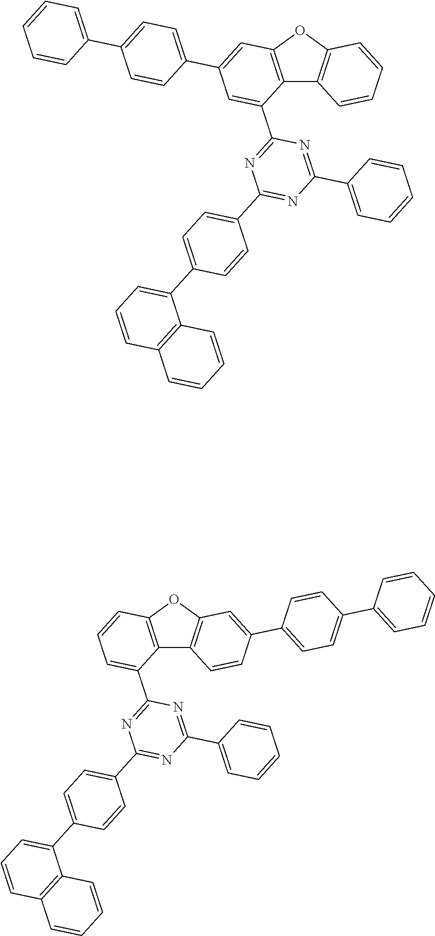 [SMILES: c1ccc(-c2ccc(-c3cc(-c4nc(-c5ccccc5)nc(-c5ccc(-c6cccc7ccccc67)cc5)n4)c4c(c3)oc3ccccc34)cc2)cc1 ; c1ccc(-c2ccc(-c3ccc4c(c3)oc3cccc(-c5nc(-c6ccccc6)nc(-c6ccc(-c7cccc8ccccc78)cc6)n5)c34)cc2)cc1]

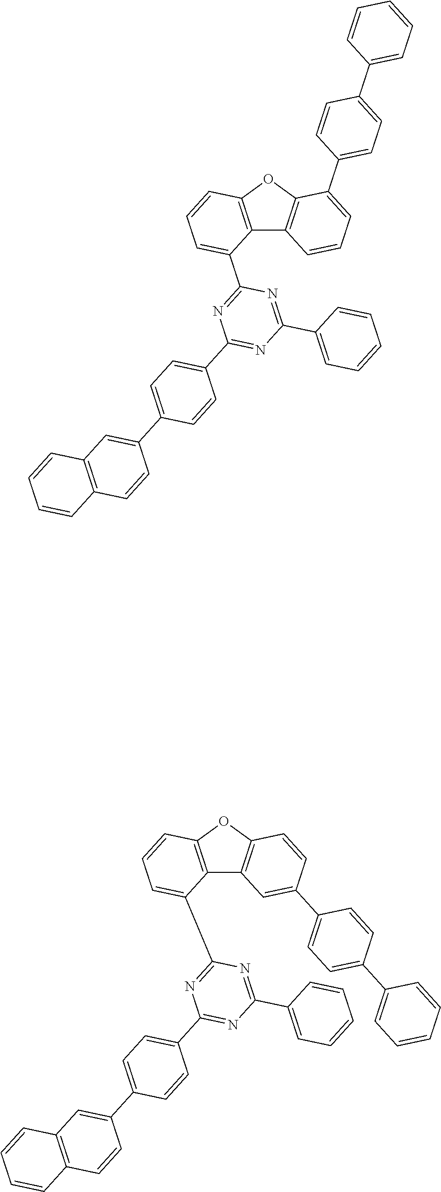 [SMILES: c1ccc(-c2ccc(-c3cccc4c3oc3cccc(-c5nc(-c6ccccc6)nc(-c6ccc(-c7ccc8ccccc8c7)cc6)n5)c34)cc2)cc1 ; c1ccc(-c2ccc(-c3ccc4oc5cccc(-c6nc(-c7ccccc7)nc(-c7ccc(-c8ccc9ccccc9c8)cc7)n6)c5c4c3)cc2)cc1]

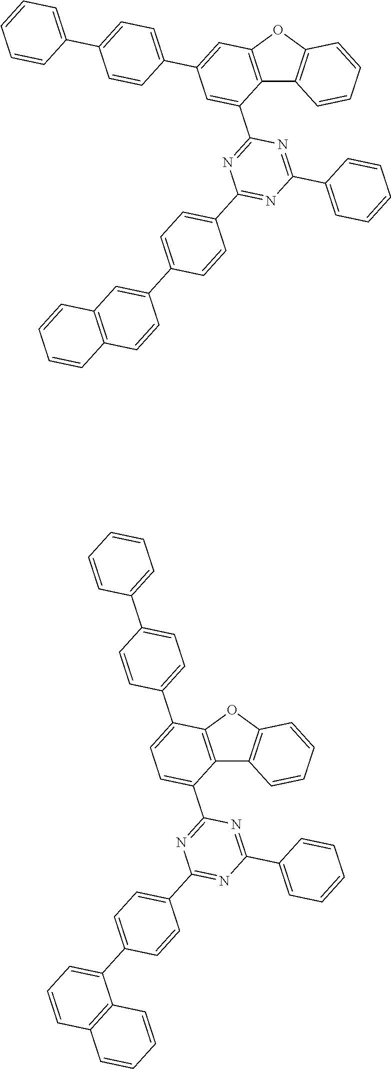 [SMILES: c1ccc(-c2ccc(-c3cc(-c4nc(-c5ccccc5)nc(-c5ccc(-c6ccc7ccccc7c6)cc5)n4)c4c(c3)oc3ccccc34)cc2)cc1 ; c1ccc(-c2ccc(-c3ccc(-c4nc(-c5ccccc5)nc(-c5ccc(-c6cccc7ccccc67)cc5)n4)c4c3oc3ccccc34)cc2)cc1]

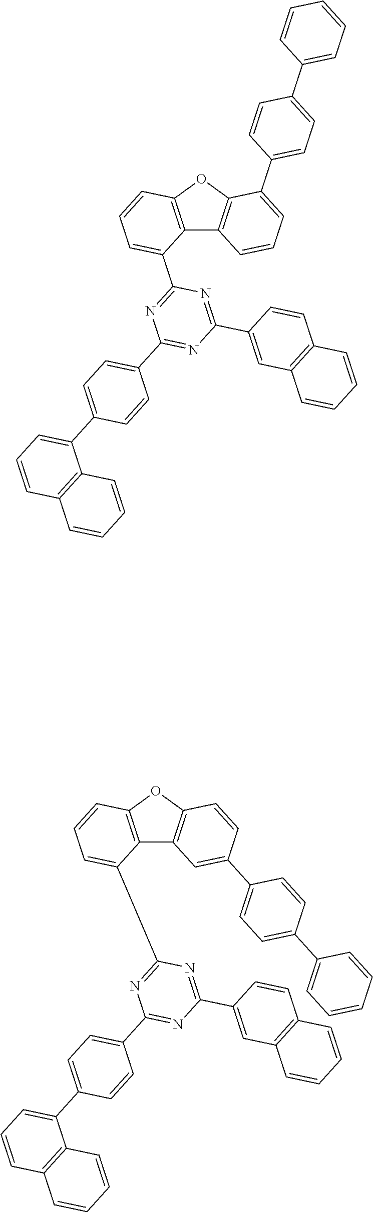 [SMILES: c1ccc(-c2ccc(-c3cccc4c3oc3cccc(-c5nc(-c6ccc(-c7cccc8ccccc78)cc6)nc(-c6ccc7ccccc7c6)n5)c34)cc2)cc1 ; c1ccc(-c2ccc(-c3ccc4oc5cccc(-c6nc(-c7ccc(-c8cccc9ccccc89)cc7)nc(-c7ccc8ccccc8c7)n6)c5c4c3)cc2)cc1]

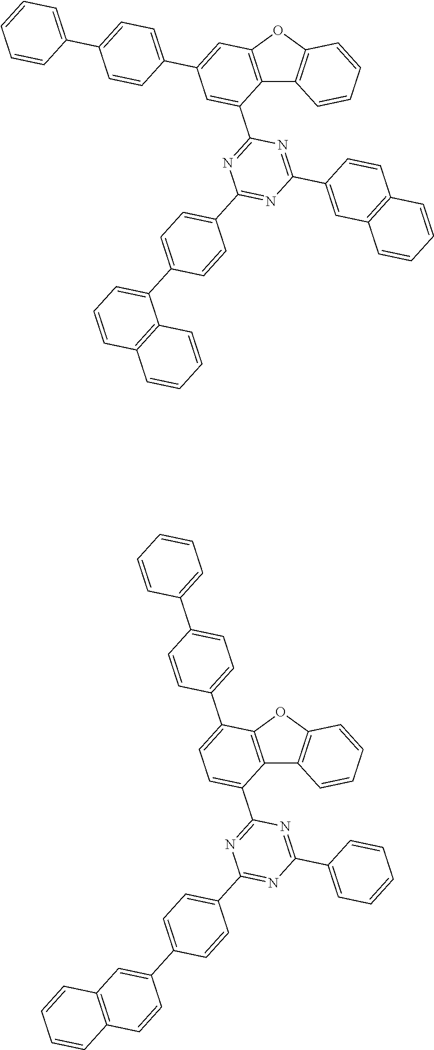 [SMILES: c1ccc(-c2ccc(-c3cc(-c4nc(-c5ccc(-c6cccc7ccccc67)cc5)nc(-c5ccc6ccccc6c5)n4)c4c(c3)oc3ccccc34)cc2)cc1 ; c1ccc(-c2ccc(-c3ccc(-c4nc(-c5ccccc5)nc(-c5ccc(-c6ccc7ccccc7c6)cc5)n4)c4c3oc3ccccc34)cc2)cc1]

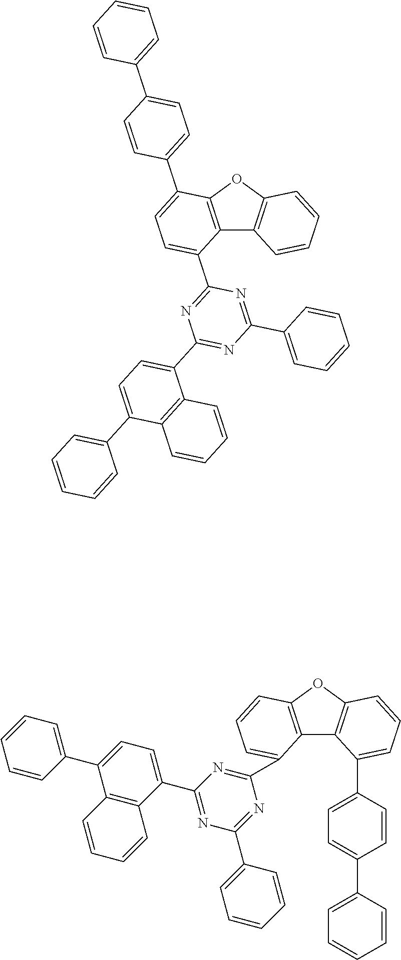 [SMILES: c1ccc(-c2ccc(-c3ccc(-c4nc(-c5ccccc5)nc(-c5ccc(-c6ccccc6)c6ccccc56)n4)c4c3oc3ccccc34)cc2)cc1 ; c1ccc(-c2ccc(-c3cccc4oc5cccc(-c6nc(-c7ccccc7)nc(-c7ccc(-c8ccccc8)c8ccccc78)n6)c5c34)cc2)cc1]

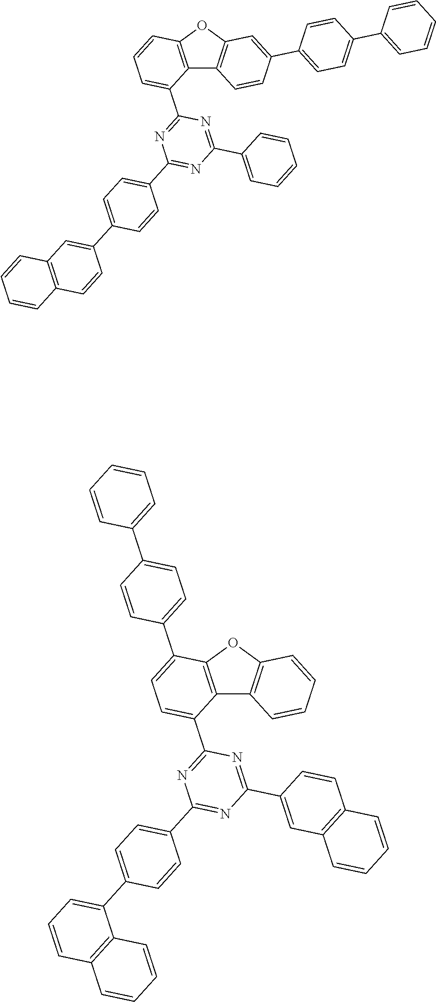 [SMILES: c1ccc(-c2ccc(-c3ccc4c(c3)oc3cccc(-c5nc(-c6ccccc6)nc(-c6ccc(-c7ccc8ccccc8c7)cc6)n5)c34)cc2)cc1 ; c1ccc(-c2ccc(-c3ccc(-c4nc(-c5ccc(-c6cccc7ccccc67)cc5)nc(-c5ccc6ccccc6c5)n4)c4c3oc3ccccc34)cc2)cc1]

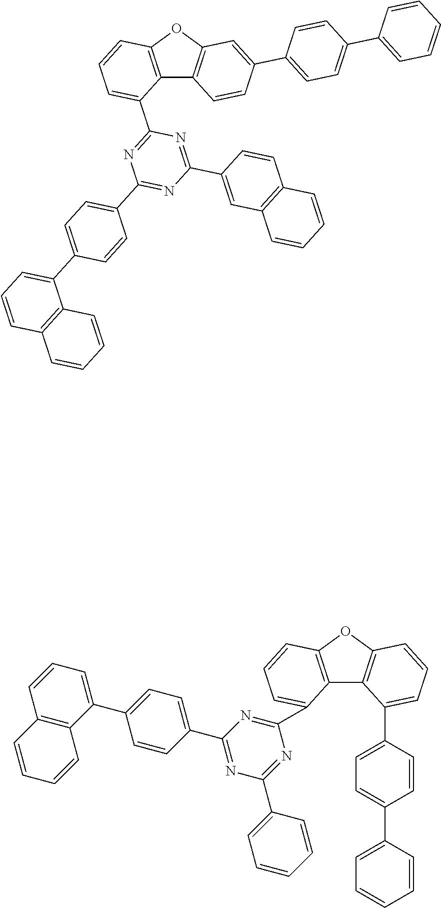 [SMILES: c1ccc(-c2ccc(-c3ccc4c(c3)oc3cccc(-c5nc(-c6ccc(-c7cccc8ccccc78)cc6)nc(-c6ccc7ccccc7c6)n5)c34)cc2)cc1 ; c1ccc(-c2ccc(-c3cccc4oc5cccc(-c6nc(-c7ccccc7)nc(-c7ccc(-c8cccc9ccccc89)cc7)n6)c5c34)cc2)cc1]

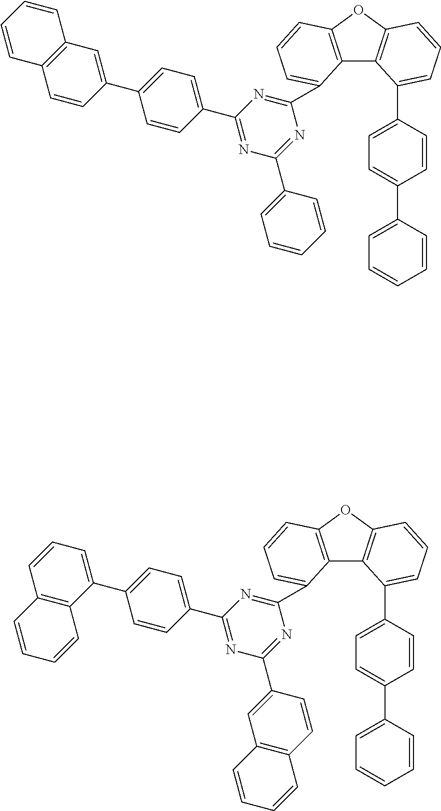 [SMILES: c1ccc(-c2ccc(-c3cccc4oc5cccc(-c6nc(-c7ccccc7)nc(-c7ccc(-c8ccc9ccccc9c8)cc7)n6)c5c34)cc2)cc1 ; c1ccc(-c2ccc(-c3cccc4oc5cccc(-c6nc(-c7ccc(-c8cccc9ccccc89)cc7)nc(-c7ccc8ccccc8c7)n6)c5c34)cc2)cc1]

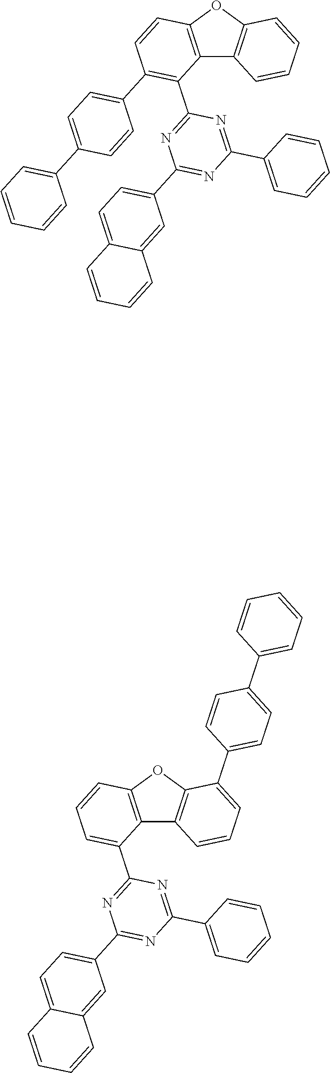 [SMILES: c1ccc(-c2ccc(-c3ccc4oc5ccccc5c4c3-c3nc(-c4ccccc4)nc(-c4ccc5ccccc5c4)n3)cc2)cc1 ; c1ccc(-c2ccc(-c3cccc4c3oc3cccc(-c5nc(-c6ccccc6)nc(-c6ccc7ccccc7c6)n5)c34)cc2)cc1]

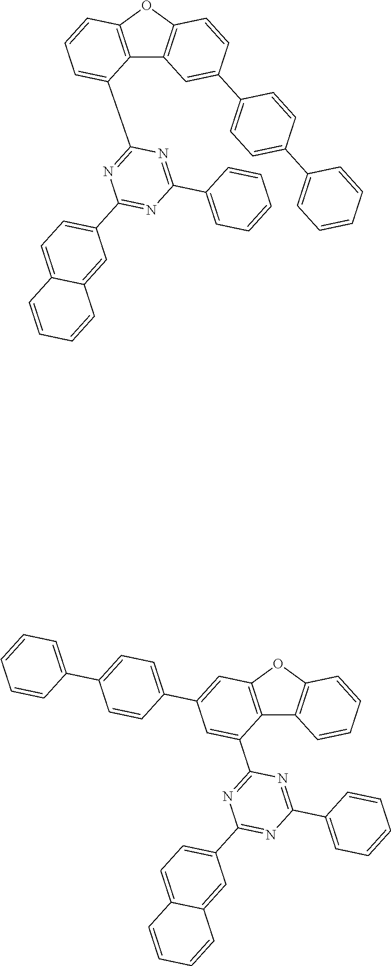 [SMILES: c1ccc(-c2ccc(-c3ccc4oc5cccc(-c6nc(-c7ccccc7)nc(-c7ccc8ccccc8c7)n6)c5c4c3)cc2)cc1 ; c1ccc(-c2ccc(-c3cc(-c4nc(-c5ccccc5)nc(-c5ccc6ccccc6c5)n4)c4c(c3)oc3ccccc34)cc2)cc1]

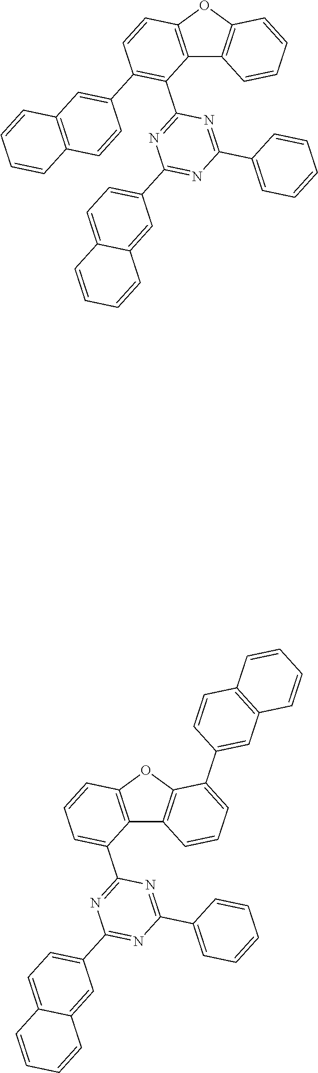 [SMILES: c1ccc(-c2nc(-c3ccc4ccccc4c3)nc(-c3c(-c4ccc5ccccc5c4)ccc4oc5ccccc5c34)n2)cc1 ; c1ccc(-c2nc(-c3ccc4ccccc4c3)nc(-c3cccc4oc5c(-c6ccc7ccccc7c6)cccc5c34)n2)cc1]

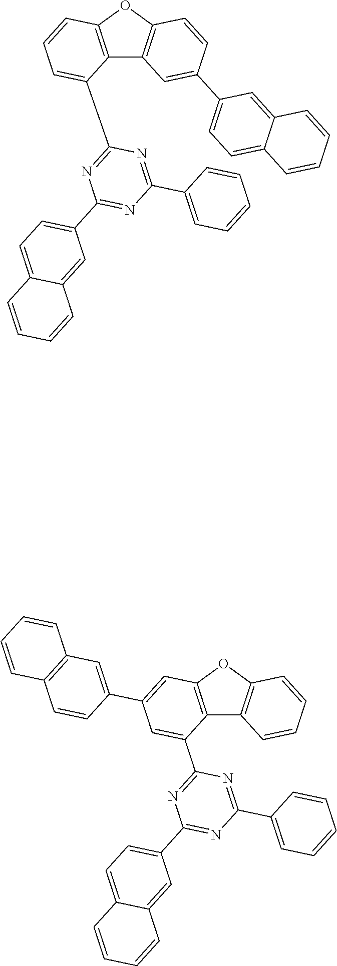 [SMILES: c1ccc(-c2nc(-c3ccc4ccccc4c3)nc(-c3cccc4oc5ccc(-c6ccc7ccccc7c6)cc5c34)n2)cc1 ; c1ccc(-c2nc(-c3ccc4ccccc4c3)nc(-c3cc(-c4ccc5ccccc5c4)cc4oc5ccccc5c34)n2)cc1]

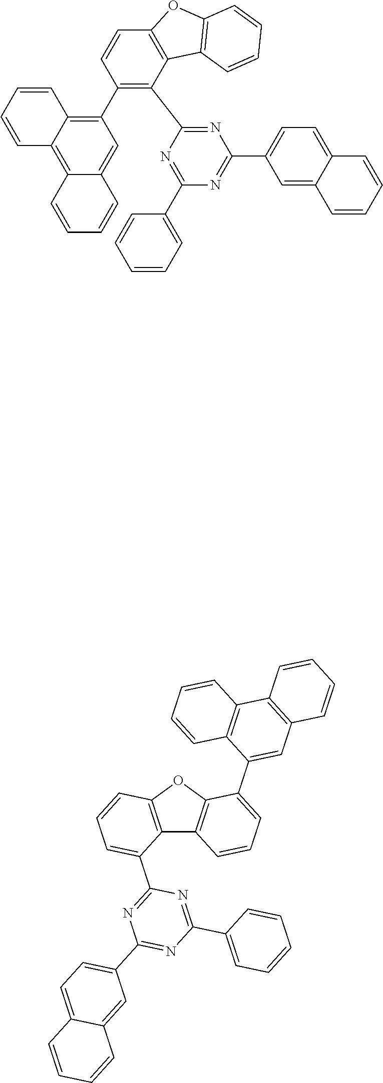 [SMILES: c1ccc(-c2nc(-c3ccc4ccccc4c3)nc(-c3c(-c4cc5ccccc5c5ccccc45)ccc4oc5ccccc5c34)n2)cc1 ; c1ccc(-c2nc(-c3ccc4ccccc4c3)nc(-c3cccc4oc5c(-c6cc7ccccc7c7ccccc67)cccc5c34)n2)cc1]

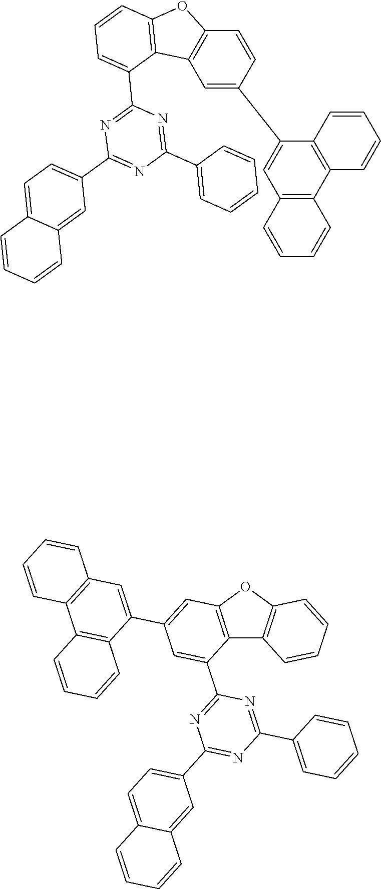 [SMILES: c1ccc(-c2nc(-c3ccc4ccccc4c3)nc(-c3cccc4oc5ccc(-c6cc7ccccc7c7ccccc67)cc5c34)n2)cc1 ; c1ccc(-c2nc(-c3ccc4ccccc4c3)nc(-c3cc(-c4cc5ccccc5c5ccccc45)cc4oc5ccccc5c34)n2)cc1]

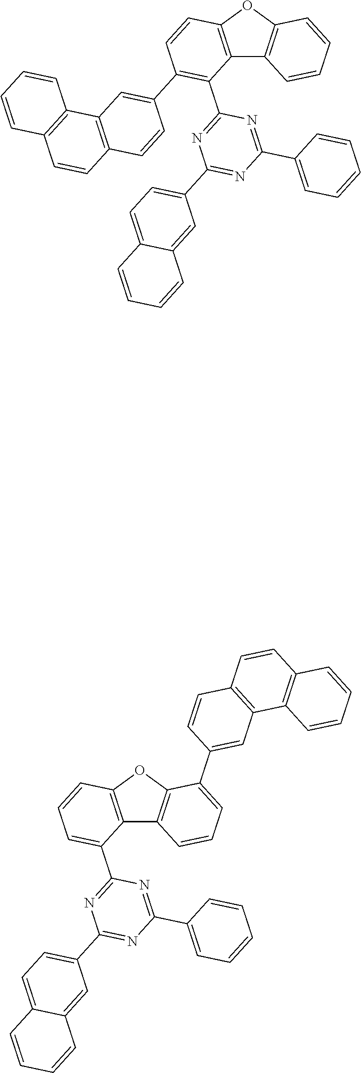 [SMILES: c1ccc(-c2nc(-c3ccc4ccccc4c3)nc(-c3c(-c4ccc5ccc6ccccc6c5c4)ccc4oc5ccccc5c34)n2)cc1 ; c1ccc(-c2nc(-c3ccc4ccccc4c3)nc(-c3cccc4oc5c(-c6ccc7ccc8ccccc8c7c6)cccc5c34)n2)cc1]

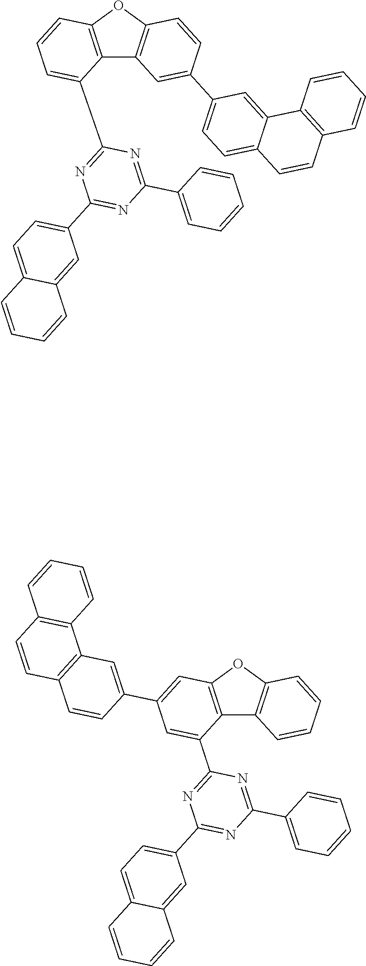 [SMILES: c1ccc(-c2nc(-c3ccc4ccccc4c3)nc(-c3cccc4oc5ccc(-c6ccc7ccc8ccccc8c7c6)cc5c34)n2)cc1 ; c1ccc(-c2nc(-c3ccc4ccccc4c3)nc(-c3cc(-c4ccc5ccc6ccccc6c5c4)cc4oc5ccccc5c34)n2)cc1]

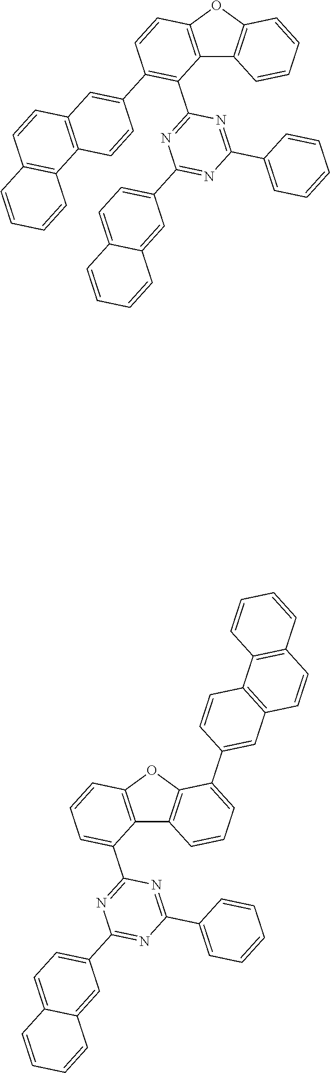 [SMILES: c1ccc(-c2nc(-c3ccc4ccccc4c3)nc(-c3c(-c4ccc5c(ccc6ccccc65)c4)ccc4oc5ccccc5c34)n2)cc1 ; c1ccc(-c2nc(-c3ccc4ccccc4c3)nc(-c3cccc4oc5c(-c6ccc7c(ccc8ccccc87)c6)cccc5c34)n2)cc1]

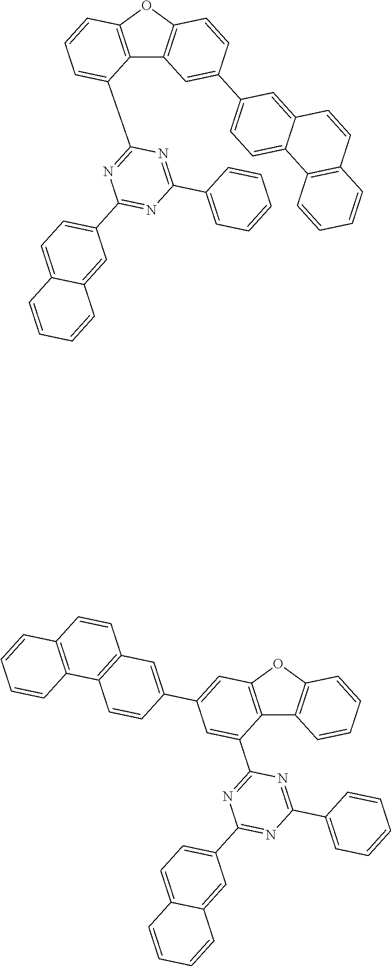 [SMILES: c1ccc(-c2nc(-c3ccc4ccccc4c3)nc(-c3cccc4oc5ccc(-c6ccc7c(ccc8ccccc87)c6)cc5c34)n2)cc1 ; c1ccc(-c2nc(-c3ccc4ccccc4c3)nc(-c3cc(-c4ccc5c(ccc6ccccc65)c4)cc4oc5ccccc5c34)n2)cc1]

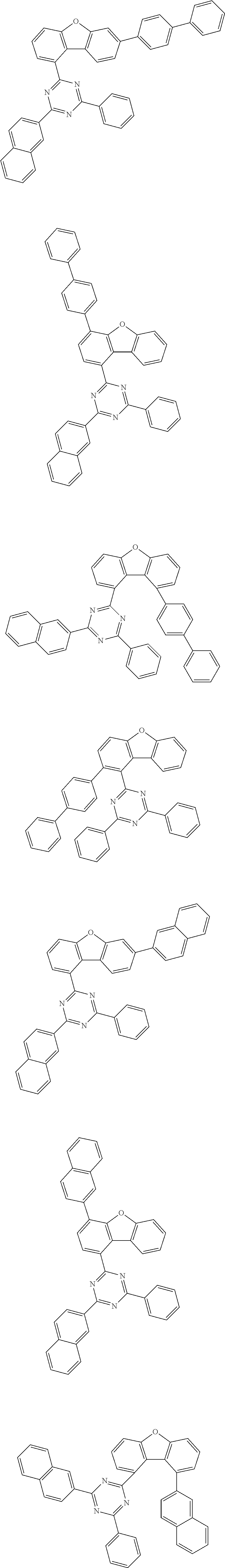

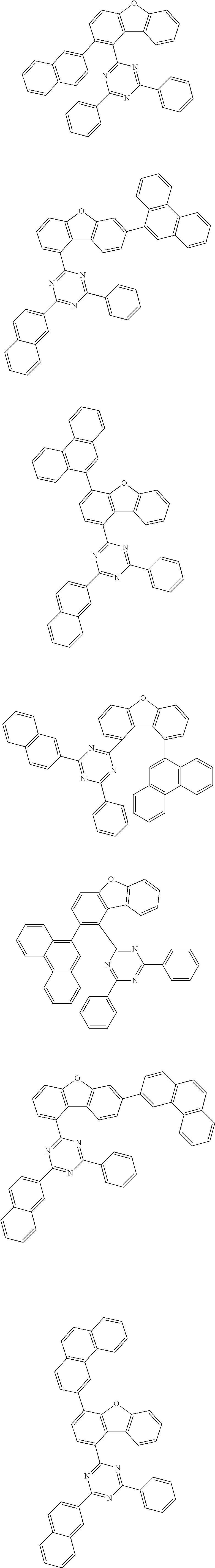

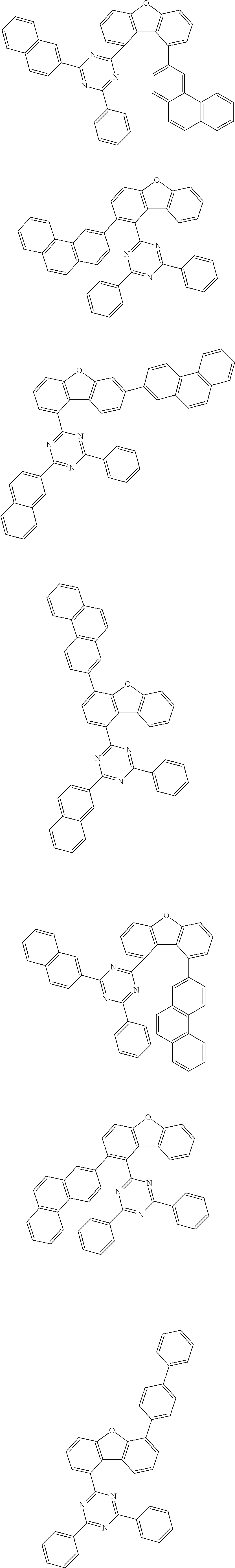

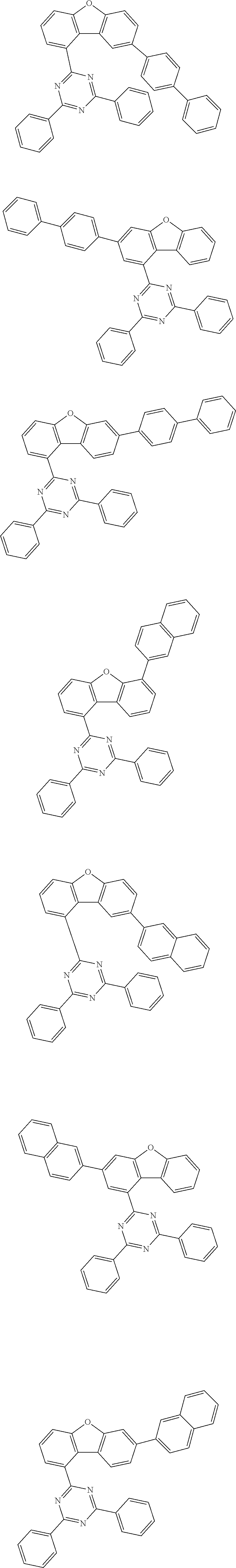

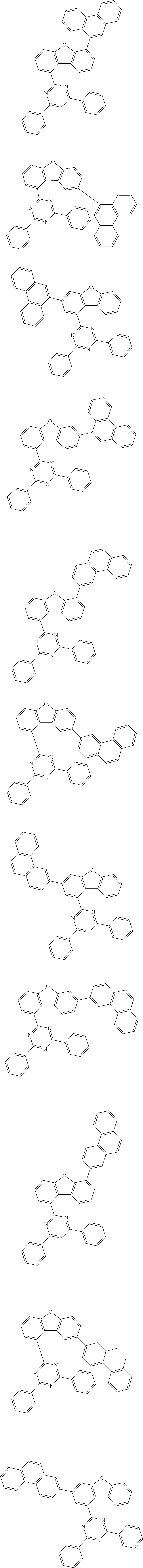

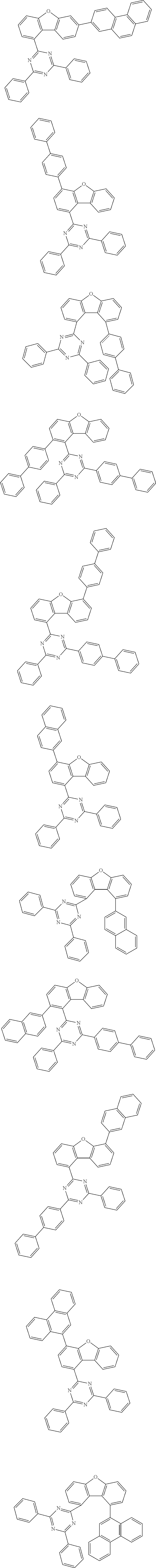

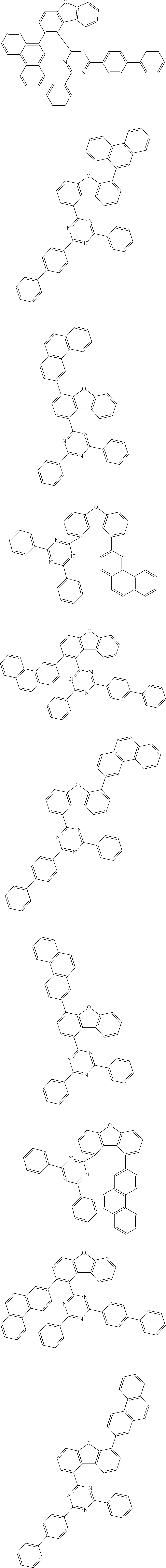

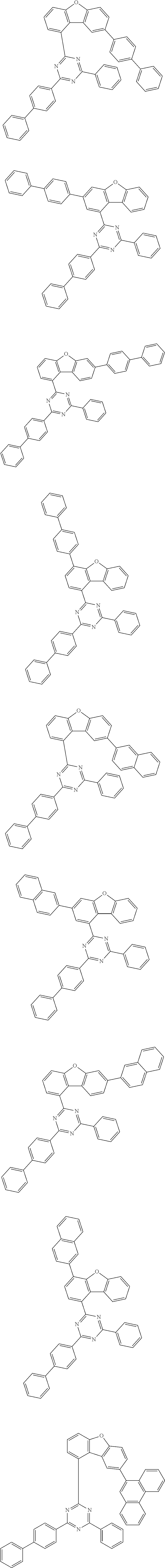

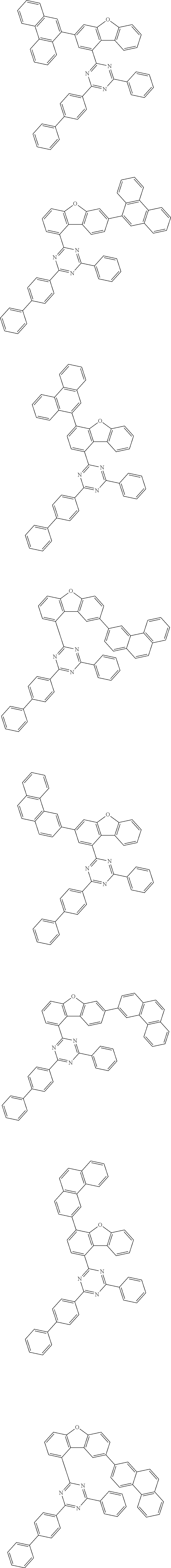

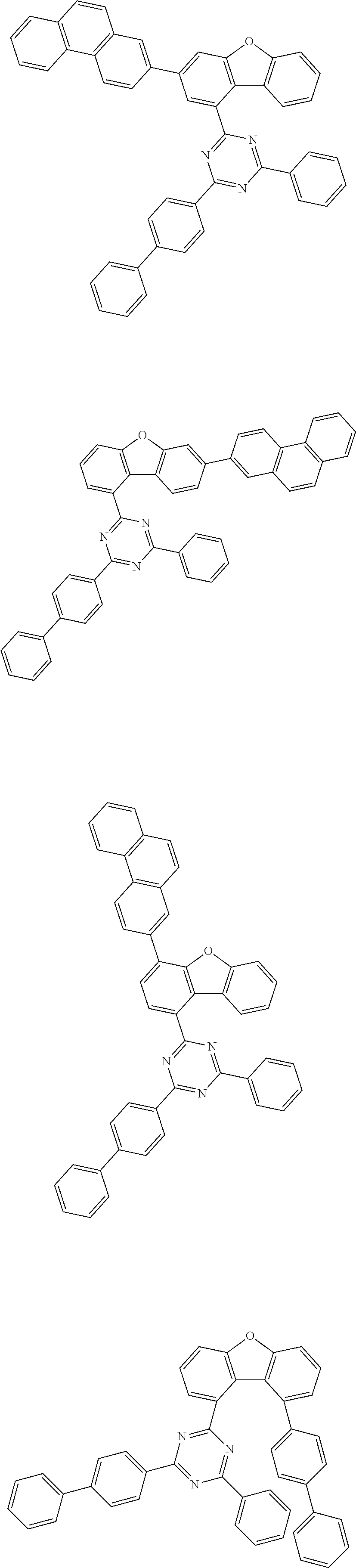 [SMILES: c1ccc(-c2ccc(-c3nc(-c4ccccc4)nc(-c4cc(-c5ccc6c(ccc7ccccc76)c5)cc5oc6ccccc6c45)n3)cc2)cc1 ; c1ccc(-c2ccc(-c3nc(-c4ccccc4)nc(-c4cccc5oc6cc(-c7ccc8c(ccc9ccccc98)c7)ccc6c45)n3)cc2)cc1 ; c1ccc(-c2ccc(-c3nc(-c4ccccc4)nc(-c4ccc(-c5ccc6c(ccc7ccccc76)c5)c5oc6ccccc6c45)n3)cc2)cc1 ; c1ccc(-c2ccc(-c3nc(-c4ccccc4)nc(-c4cccc5oc6cccc(-c7ccc(-c8ccccc8)cc7)c6c45)n3)cc2)cc1]

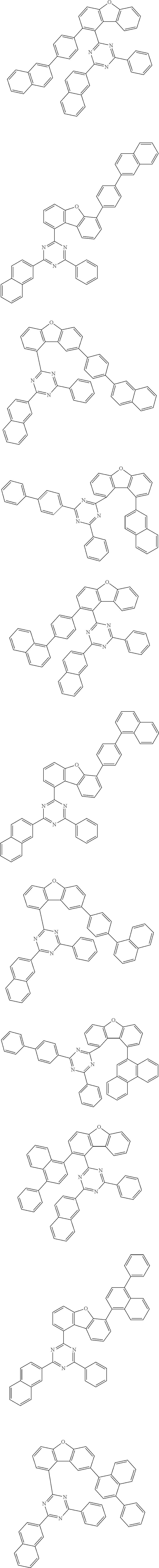

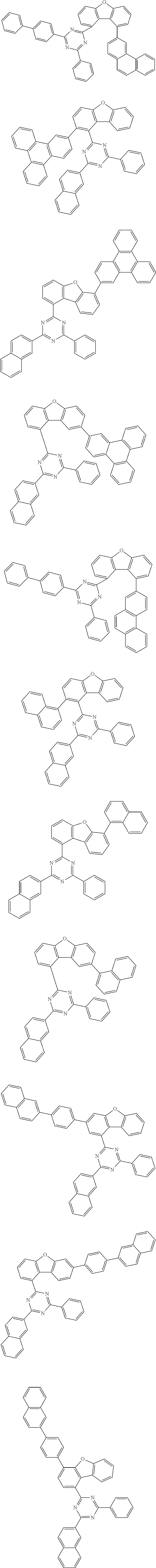

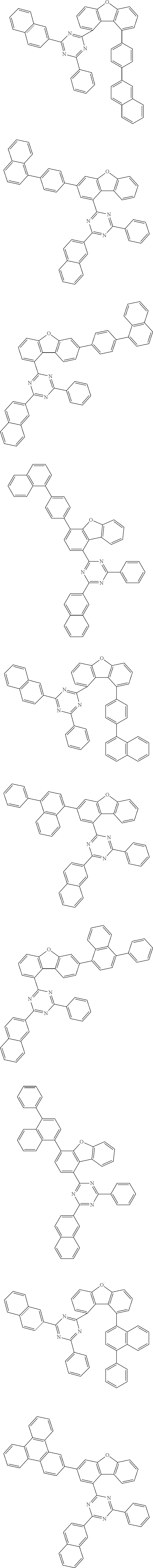

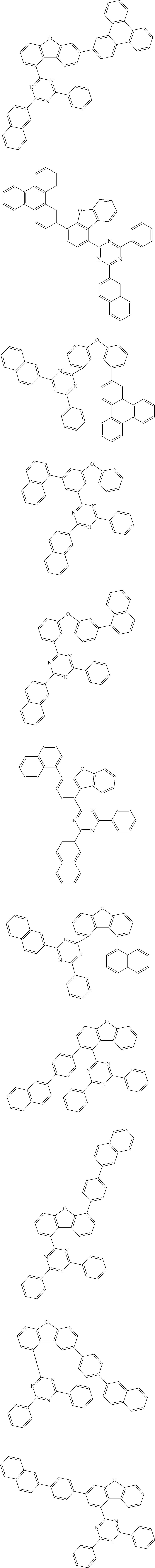

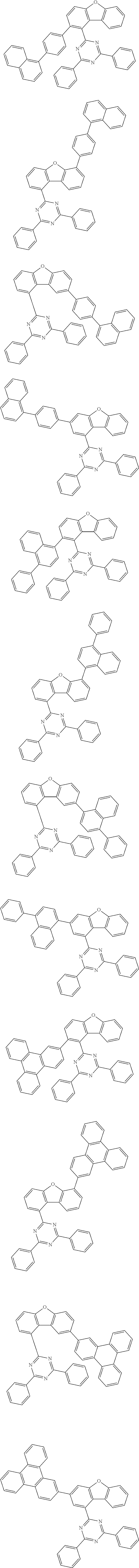

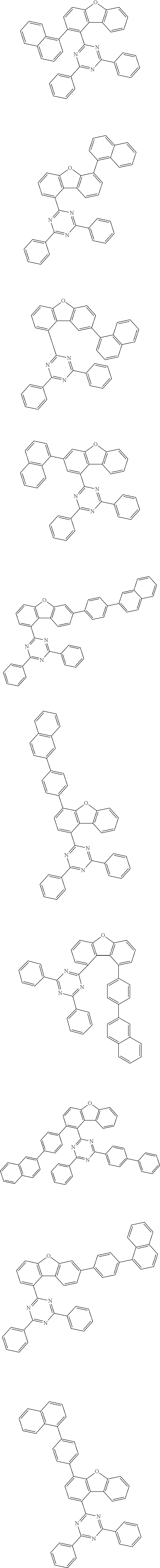

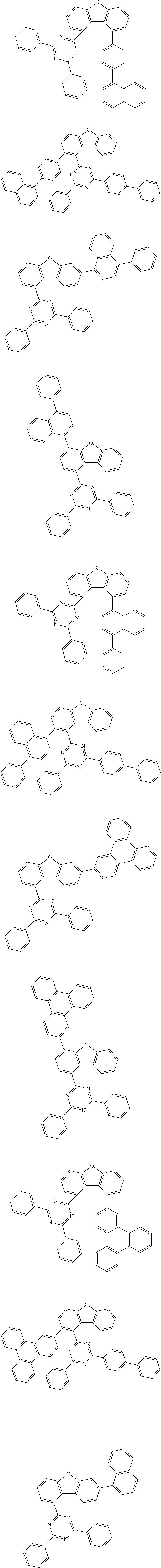

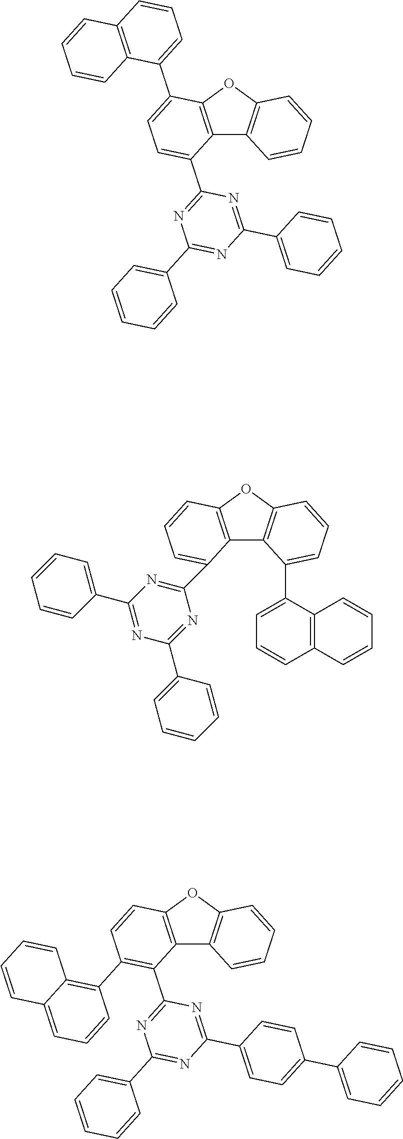 [SMILES: c1ccc(-c2nc(-c3ccccc3)nc(-c3ccc(-c4cccc5ccccc45)c4oc5ccccc5c34)n2)cc1 ; c1ccc(-c2nc(-c3ccccc3)nc(-c3cccc4oc5cccc(-c6cccc7ccccc67)c5c34)n2)cc1 ; c1ccc(-c2ccc(-c3nc(-c4ccccc4)nc(-c4c(-c5cccc6ccccc56)ccc5oc6ccccc6c45)n3)cc2)cc1]

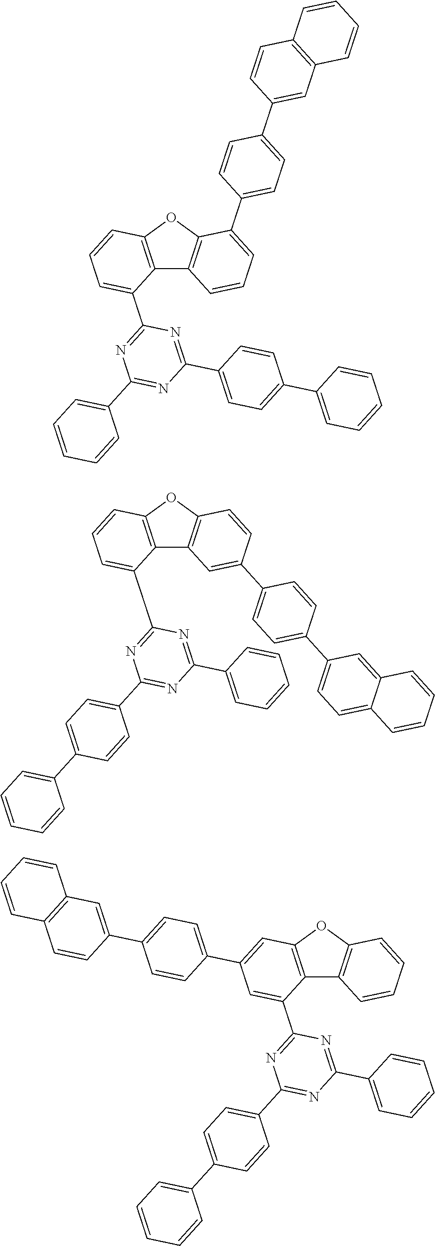

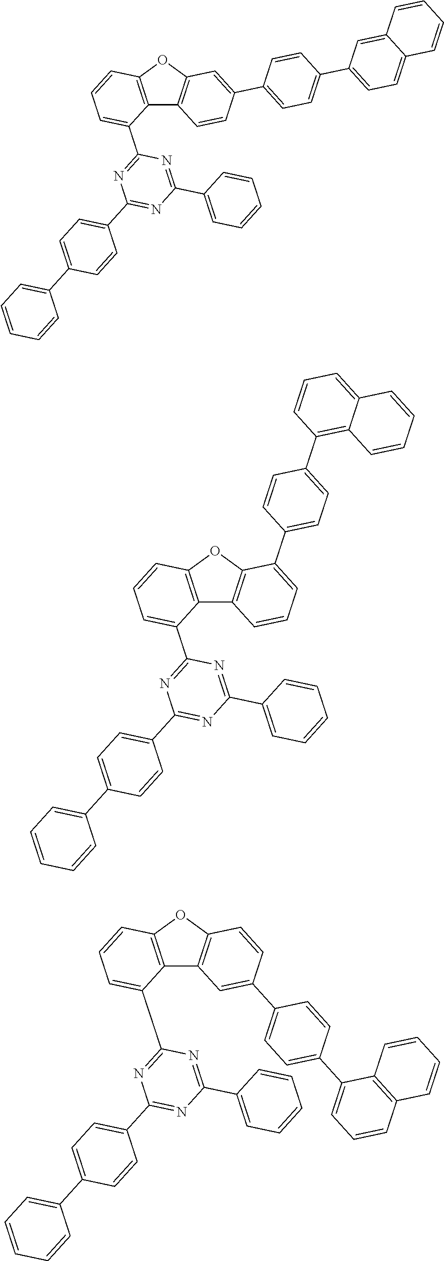 [SMILES: c1ccc(-c2ccc(-c3nc(-c4ccccc4)nc(-c4cccc5oc6cc(-c7ccc(-c8ccc9ccccc9c8)cc7)ccc6c45)n3)cc2)cc1 ; c1ccc(-c2ccc(-c3nc(-c4ccccc4)nc(-c4cccc5oc6c(-c7ccc(-c8cccc9ccccc89)cc7)cccc6c45)n3)cc2)cc1 ; c1ccc(-c2ccc(-c3nc(-c4ccccc4)nc(-c4cccc5oc6ccc(-c7ccc(-c8cccc9ccccc89)cc7)cc6c45)n3)cc2)cc1]

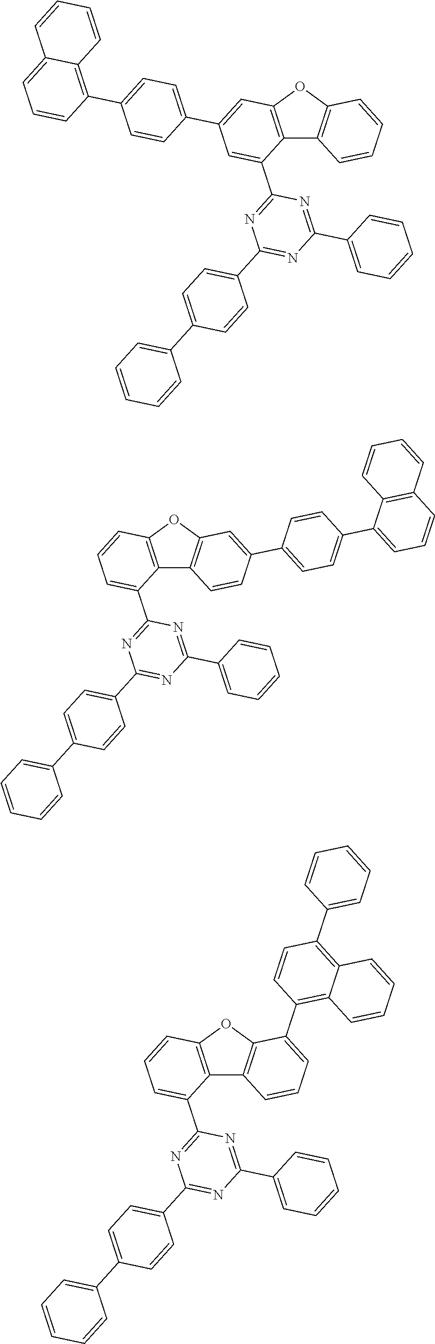 [SMILES: c1ccc(-c2ccc(-c3nc(-c4ccccc4)nc(-c4cc(-c5ccc(-c6cccc7ccccc67)cc5)cc5oc6ccccc6c45)n3)cc2)cc1 ; c1ccc(-c2ccc(-c3nc(-c4ccccc4)nc(-c4cccc5oc6cc(-c7ccc(-c8cccc9ccccc89)cc7)ccc6c45)n3)cc2)cc1 ; c1ccc(-c2ccc(-c3nc(-c4ccccc4)nc(-c4cccc5oc6c(-c7ccc(-c8ccccc8)c8ccccc78)cccc6c45)n3)cc2)cc1]

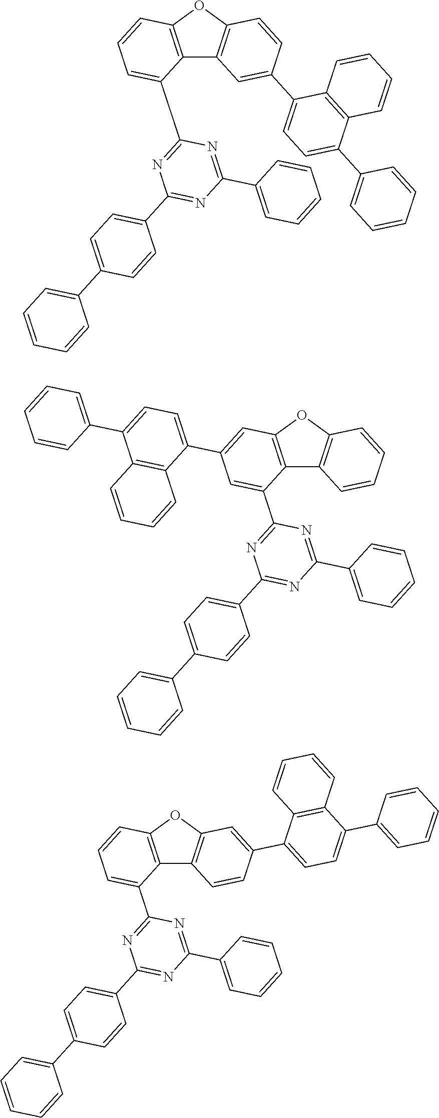

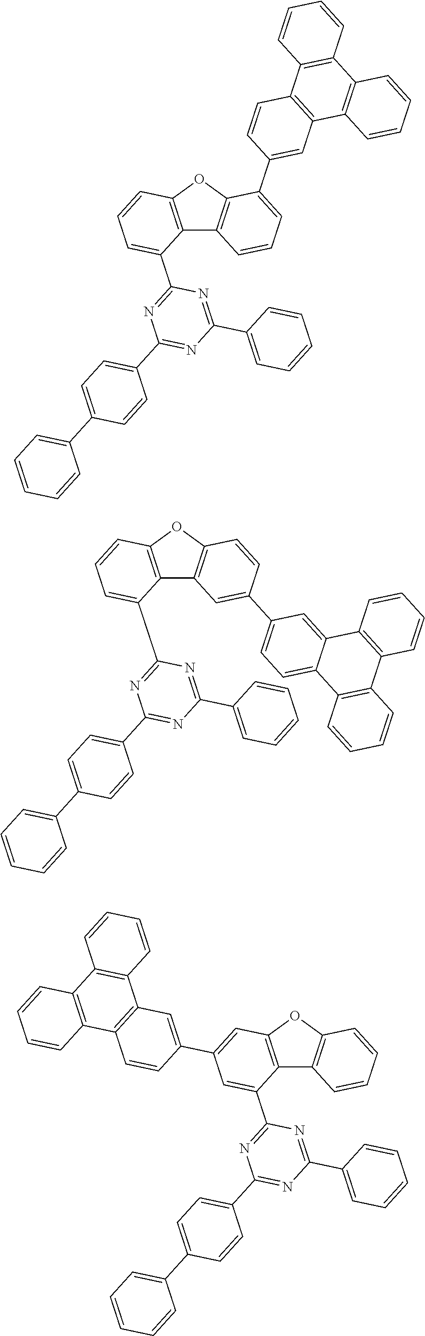 [SMILES: c1ccc(-c2ccc(-c3nc(-c4ccccc4)nc(-c4cccc5oc6c(-c7ccc8c9ccccc9c9ccccc9c8c7)cccc6c45)n3)cc2)cc1 ; c1ccc(-c2ccc(-c3nc(-c4ccccc4)nc(-c4cccc5oc6ccc(-c7ccc8c9ccccc9c9ccccc9c8c7)cc6c45)n3)cc2)cc1 ; c1ccc(-c2ccc(-c3nc(-c4ccccc4)nc(-c4cc(-c5ccc6c7ccccc7c7ccccc7c6c5)cc5oc6ccccc6c45)n3)cc2)cc1]

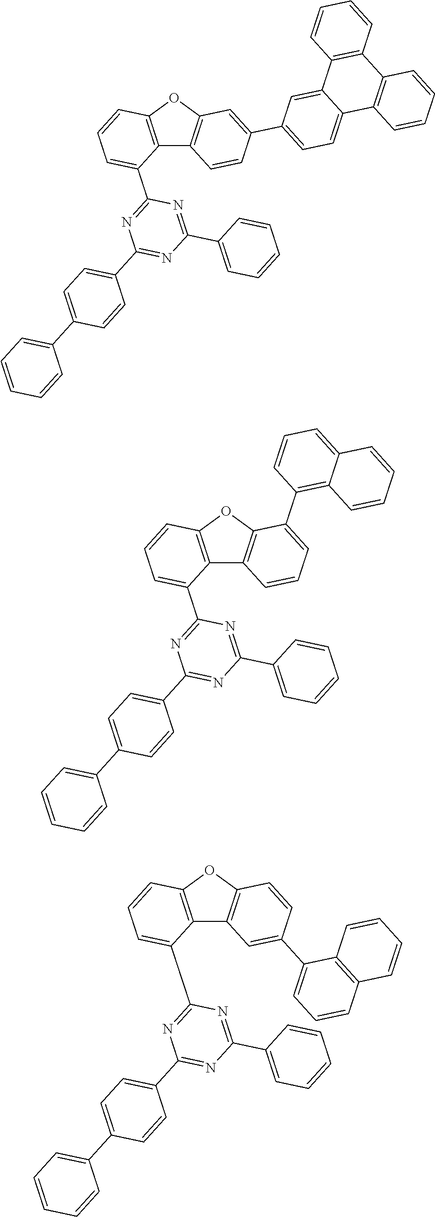 [SMILES: c1ccc(-c2ccc(-c3nc(-c4ccccc4)nc(-c4cccc5oc6cc(-c7ccc8c9ccccc9c9ccccc9c8c7)ccc6c45)n3)cc2)cc1 ; c1ccc(-c2ccc(-c3nc(-c4ccccc4)nc(-c4cccc5oc6c(-c7cccc8ccccc78)cccc6c45)n3)cc2)cc1 ; c1ccc(-c2ccc(-c3nc(-c4ccccc4)nc(-c4cccc5oc6ccc(-c7cccc8ccccc78)cc6c45)n3)cc2)cc1]

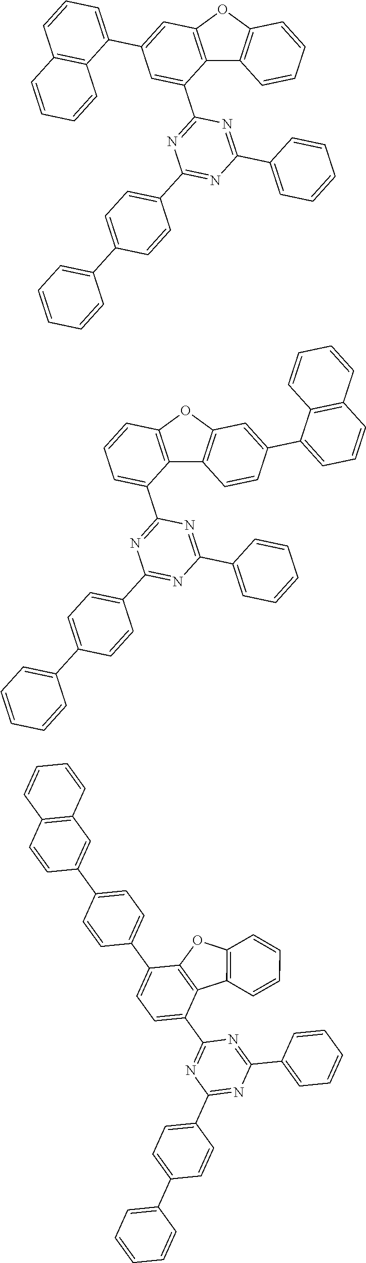

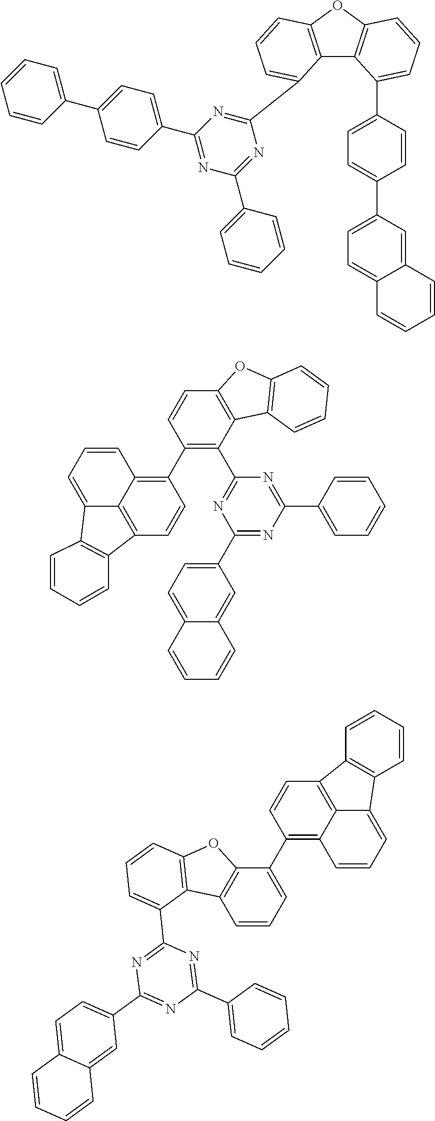 [SMILES: c1ccc(-c2ccc(-c3nc(-c4ccccc4)nc(-c4cccc5oc6cccc(-c7ccc(-c8ccc9ccccc9c8)cc7)c6c45)n3)cc2)cc1 ; c1ccc(-c2nc(-c3ccc4ccccc4c3)nc(-c3c(-c4ccc5c6c(cccc46)-c4ccccc4-5)ccc4oc5ccccc5c34)n2)cc1 ; c1ccc(-c2nc(-c3ccc4ccccc4c3)nc(-c3cccc4oc5c(-c6ccc7c8c(cccc68)-c6ccccc6-7)cccc5c34)n2)cc1]

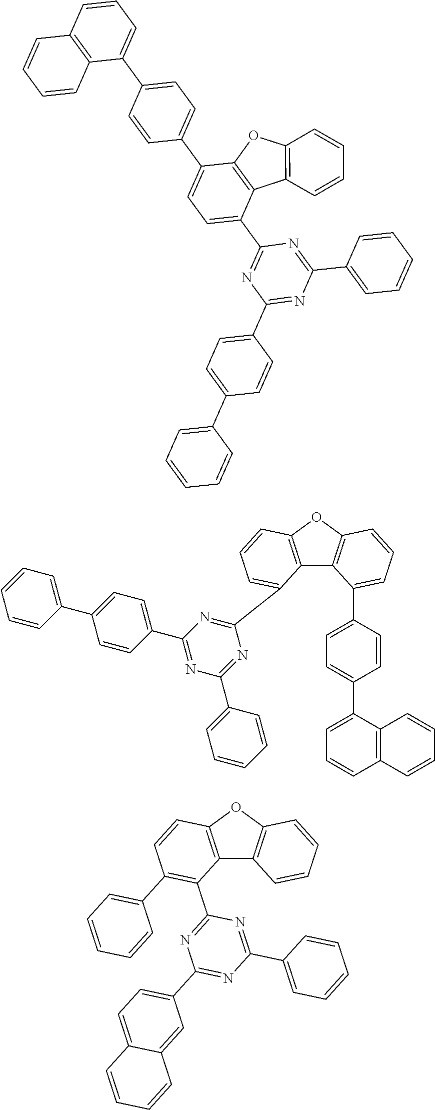

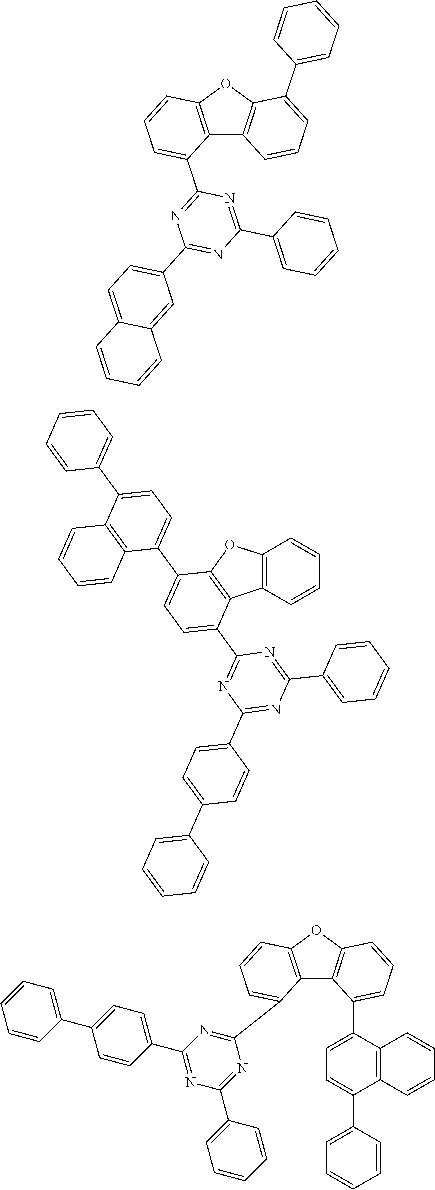

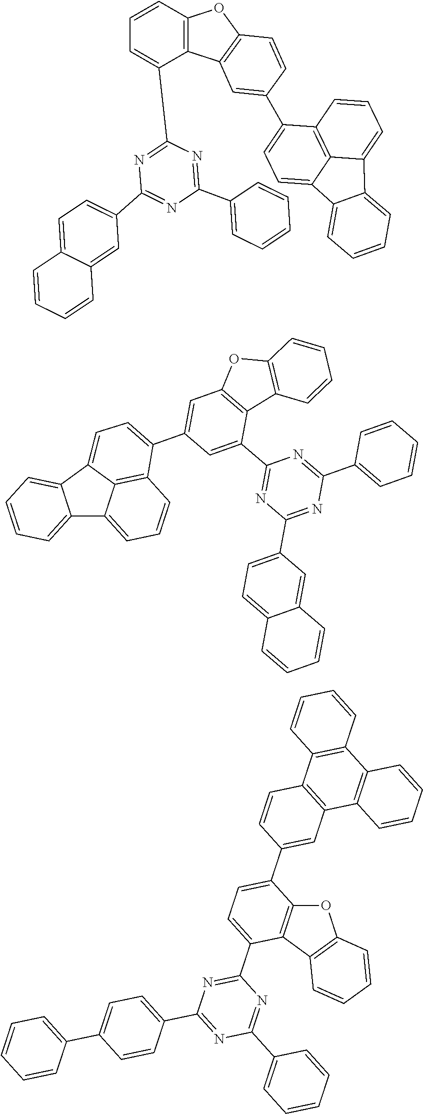

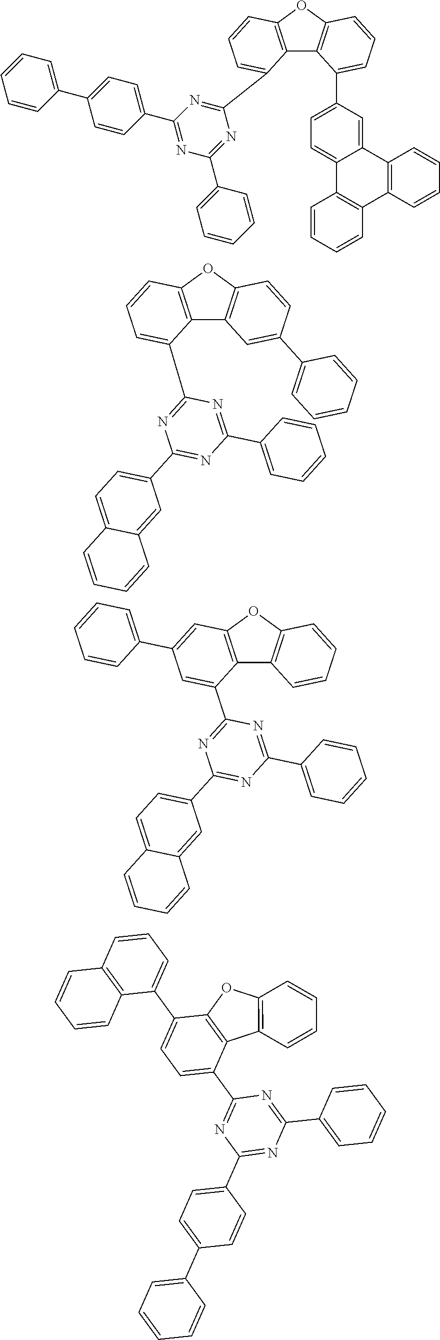

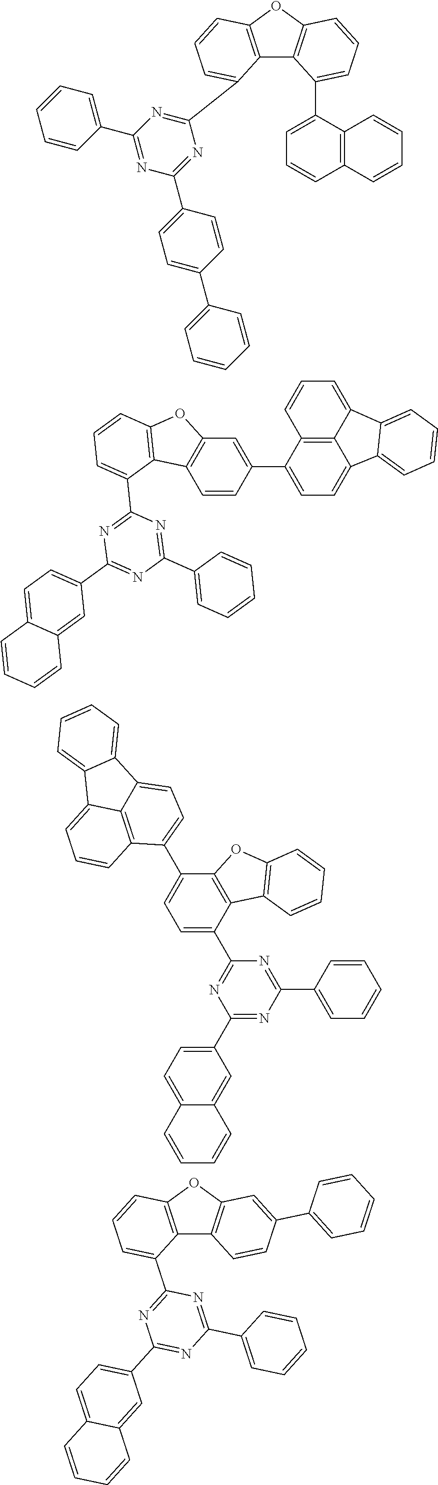

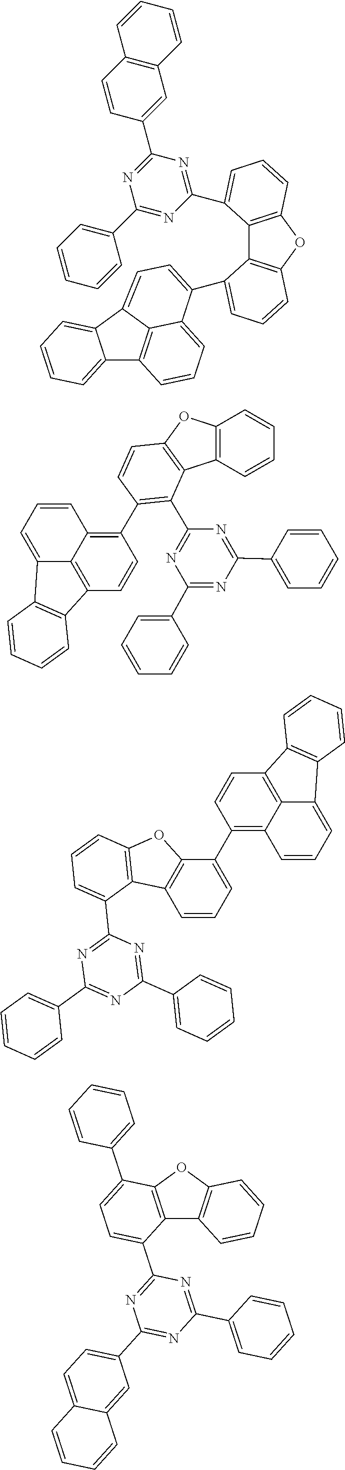

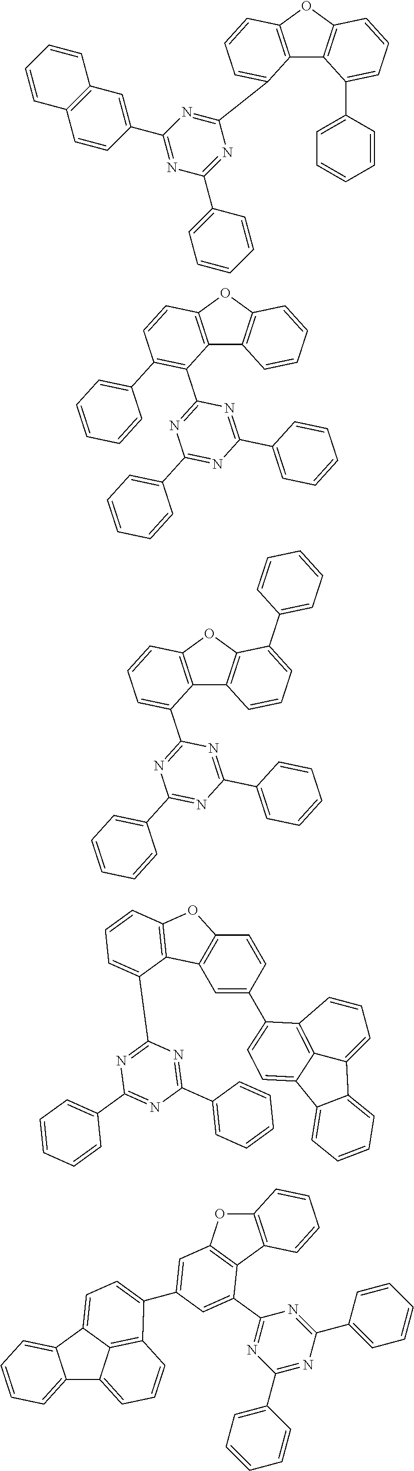

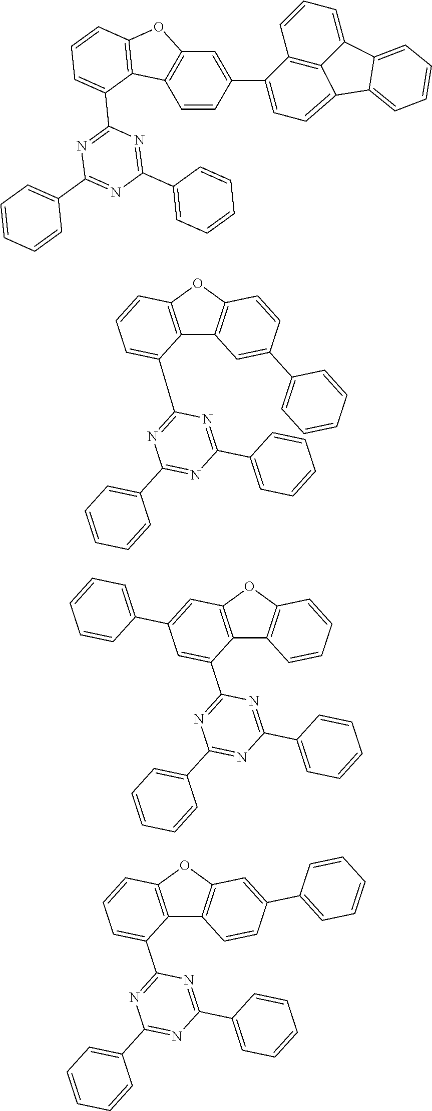

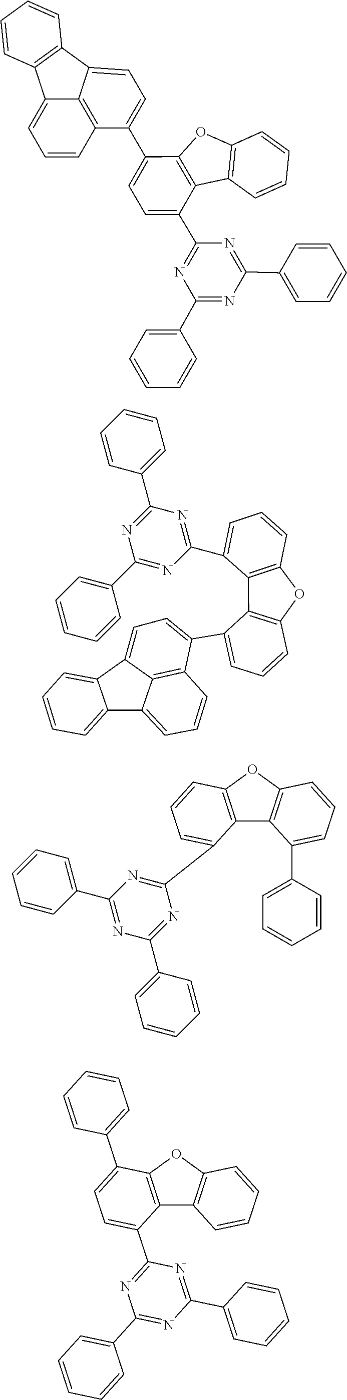

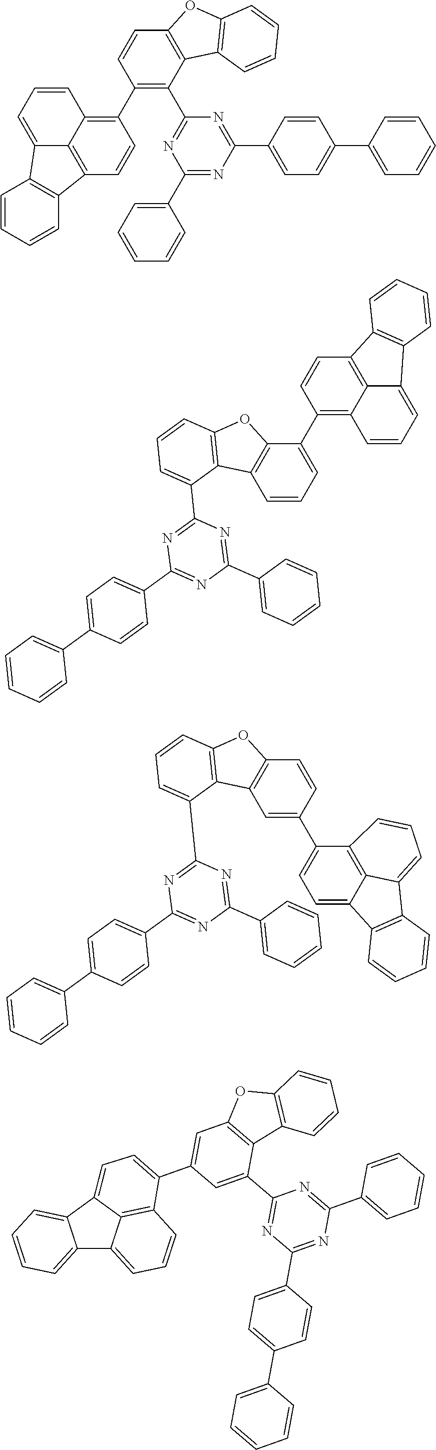 [SMILES: c1ccc(-c2ccc(-c3nc(-c4ccccc4)nc(-c4c(-c5ccc6c7c(cccc57)-c5ccccc5-6)ccc5oc6ccccc6c45)n3)cc2)cc1 ; c1ccc(-c2ccc(-c3nc(-c4ccccc4)nc(-c4cccc5oc6c(-c7ccc8c9c(cccc79)-c7ccccc7-8)cccc6c45)n3)cc2)cc1 ; c1ccc(-c2ccc(-c3nc(-c4ccccc4)nc(-c4cccc5oc6ccc(-c7ccc8c9c(cccc79)-c7ccccc7-8)cc6c45)n3)cc2)cc1 ; c1ccc(-c2ccc(-c3nc(-c4ccccc4)nc(-c4cc(-c5ccc6c7c(cccc57)-c5ccccc5-6)cc5oc6ccccc6c45)n3)cc2)cc1]

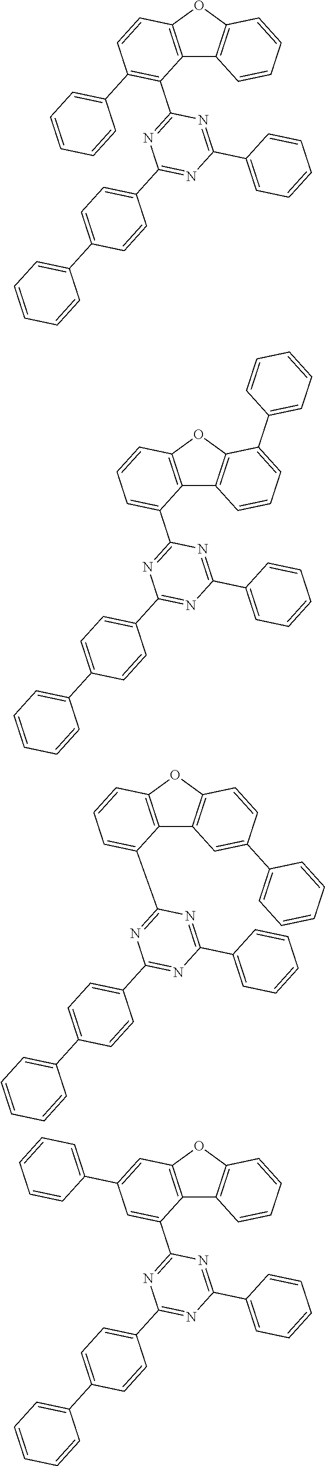

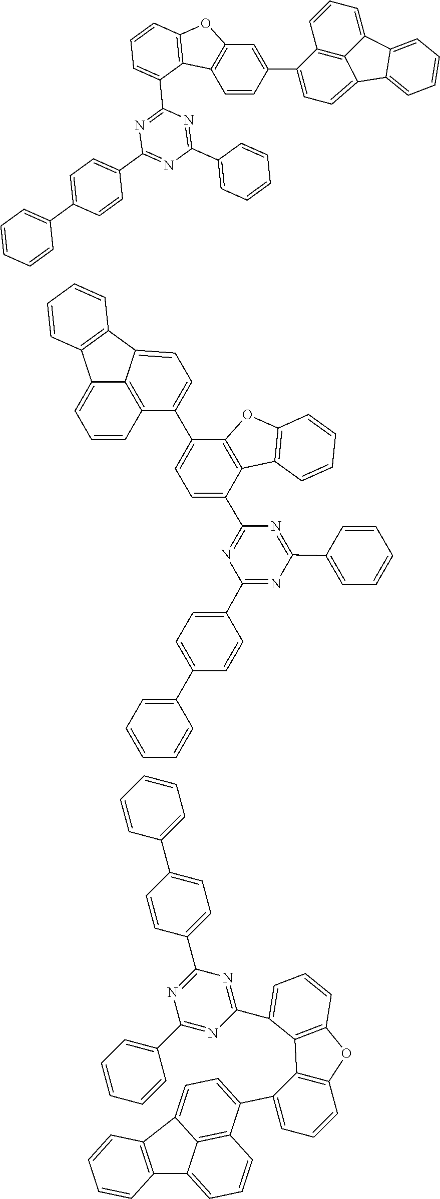

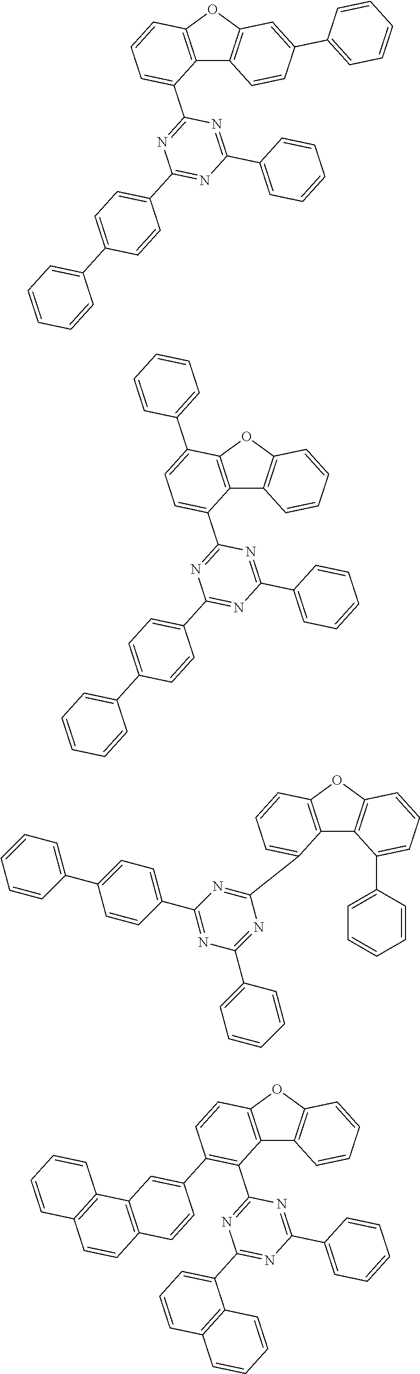

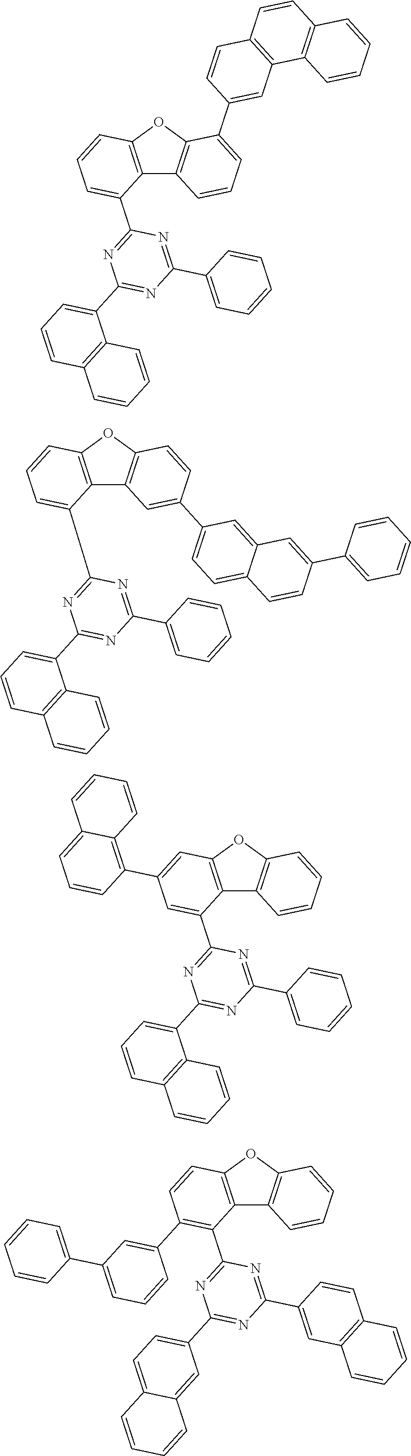

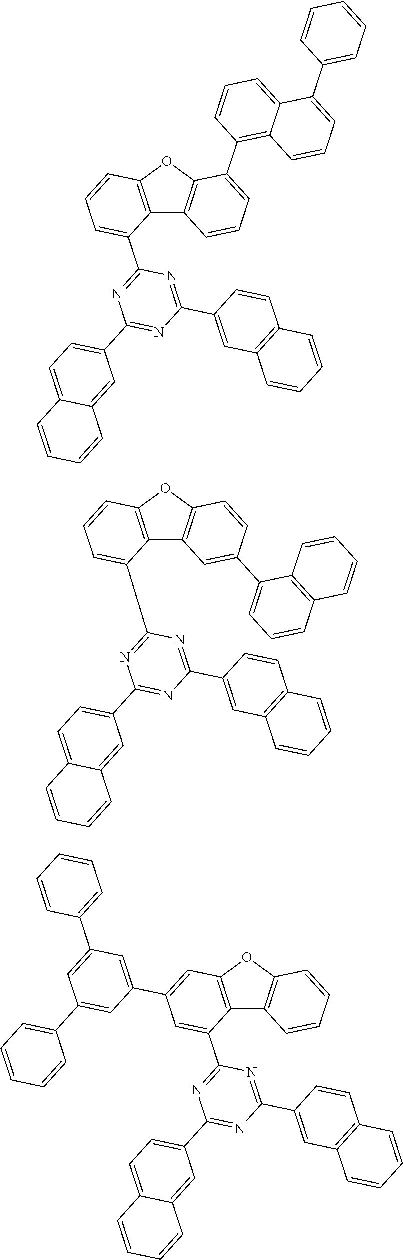

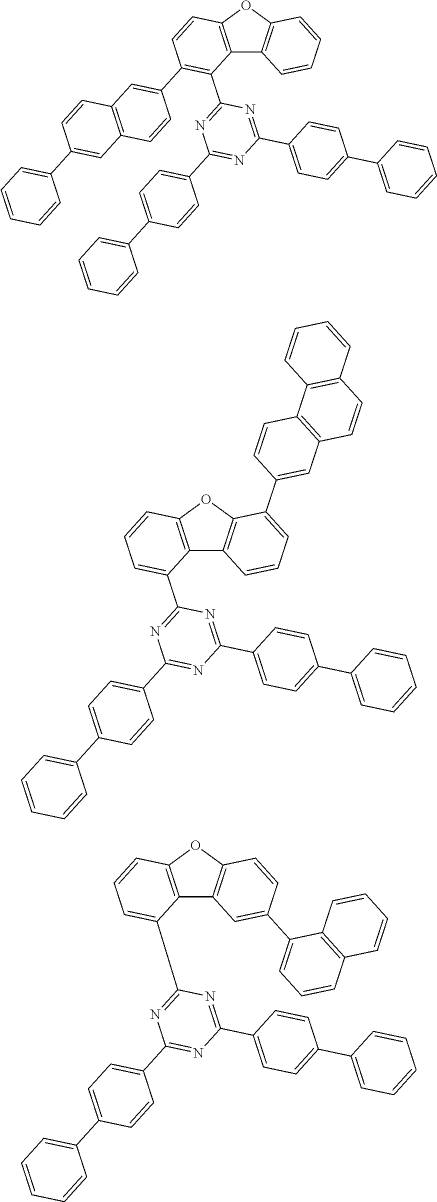 [SMILES: c1ccc(-c2ccc(-c3nc(-c4ccc(-c5ccccc5)cc4)nc(-c4c(-c5ccc6cc(-c7ccccc7)ccc6c5)ccc5oc6ccccc6c45)n3)cc2)cc1 ; c1ccc(-c2ccc(-c3nc(-c4ccc(-c5ccccc5)cc4)nc(-c4cccc5oc6c(-c7ccc8c(ccc9ccccc98)c7)cccc6c45)n3)cc2)cc1 ; c1ccc(-c2ccc(-c3nc(-c4ccc(-c5ccccc5)cc4)nc(-c4cccc5oc6ccc(-c7cccc8ccccc78)cc6c45)n3)cc2)cc1]

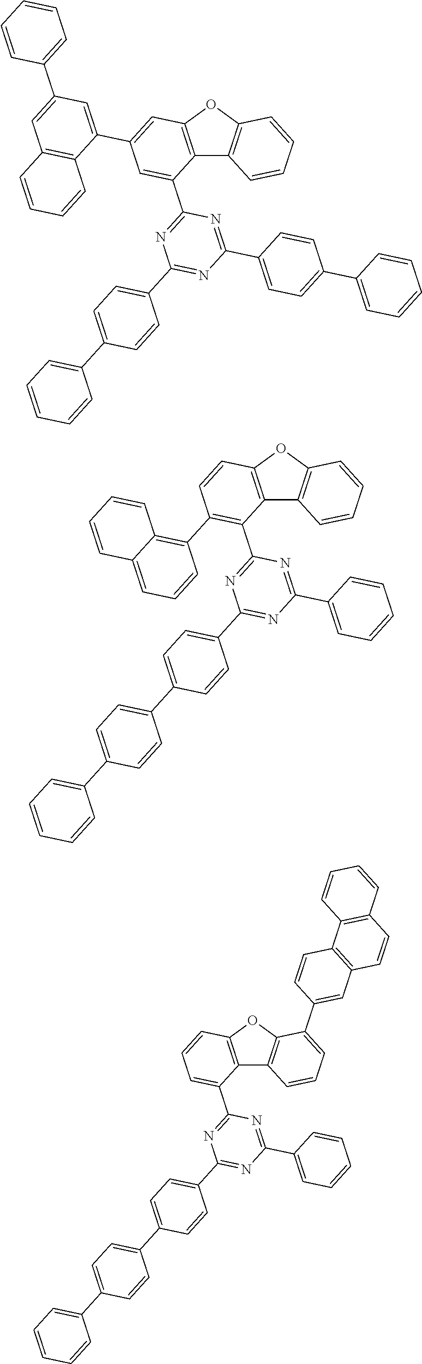 [SMILES: c1ccc(-c2ccc(-c3nc(-c4ccc(-c5ccccc5)cc4)nc(-c4cc(-c5cc(-c6ccccc6)cc6ccccc56)cc5oc6ccccc6c45)n3)cc2)cc1 ; c1ccc(-c2ccc(-c3ccc(-c4nc(-c5ccccc5)nc(-c5c(-c6cccc7ccccc67)ccc6oc7ccccc7c56)n4)cc3)cc2)cc1 ; c1ccc(-c2ccc(-c3ccc(-c4nc(-c5ccccc5)nc(-c5cccc6oc7c(-c8ccc9c(ccc%10ccccc%109)c8)cccc7c56)n4)cc3)cc2)cc1]

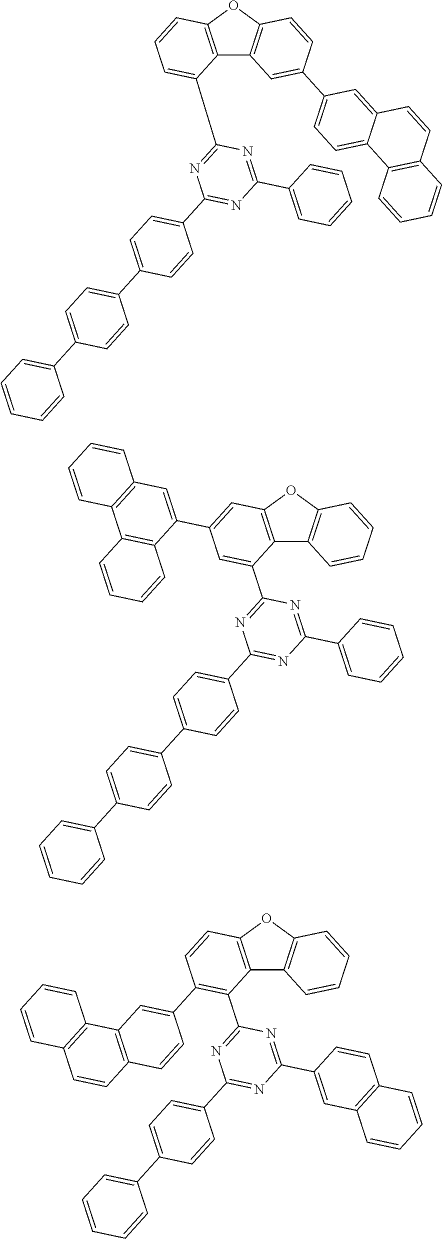 [SMILES: c1ccc(-c2ccc(-c3ccc(-c4nc(-c5ccccc5)nc(-c5cccc6oc7ccc(-c8ccc9c(ccc%10ccccc%109)c8)cc7c56)n4)cc3)cc2)cc1 ; c1ccc(-c2ccc(-c3ccc(-c4nc(-c5ccccc5)nc(-c5cc(-c6cc7ccccc7c7ccccc67)cc6oc7ccccc7c56)n4)cc3)cc2)cc1 ; c1ccc(-c2ccc(-c3nc(-c4ccc5ccccc5c4)nc(-c4c(-c5ccc6ccc7ccccc7c6c5)ccc5oc6ccccc6c45)n3)cc2)cc1]

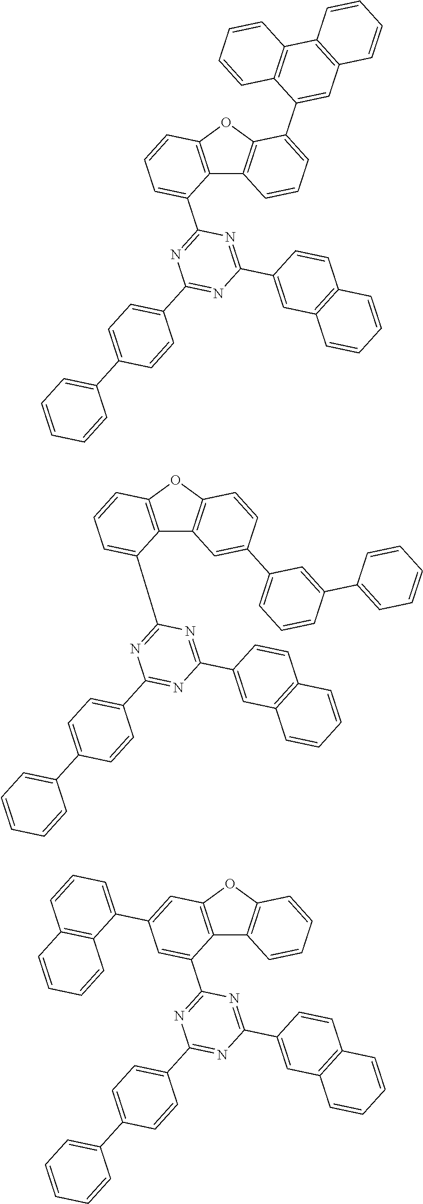 [SMILES: c1ccc(-c2ccc(-c3nc(-c4ccc5ccccc5c4)nc(-c4cccc5oc6c(-c7cc8ccccc8c8ccccc78)cccc6c45)n3)cc2)cc1 ; c1ccc(-c2ccc(-c3nc(-c4ccc5ccccc5c4)nc(-c4cccc5oc6ccc(-c7cccc(-c8ccccc8)c7)cc6c45)n3)cc2)cc1 ; c1ccc(-c2ccc(-c3nc(-c4ccc5ccccc5c4)nc(-c4cc(-c5cccc6ccccc56)cc5oc6ccccc6c45)n3)cc2)cc1]

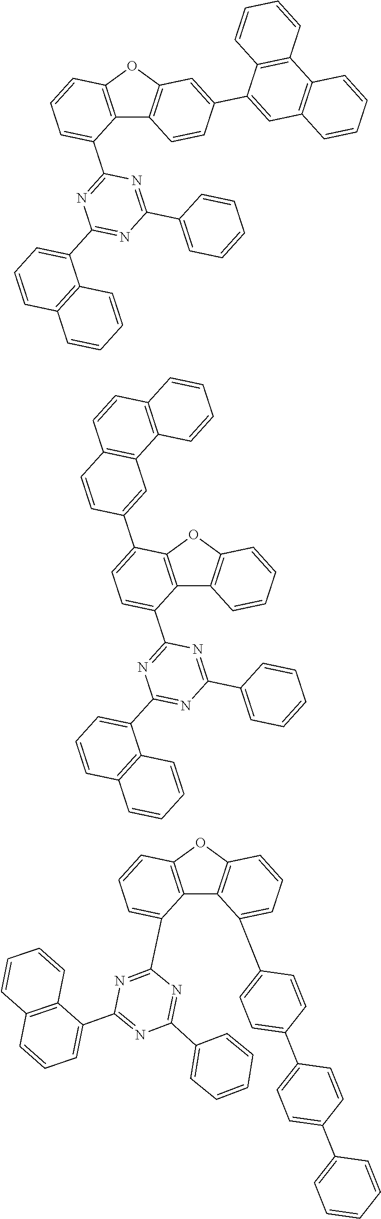

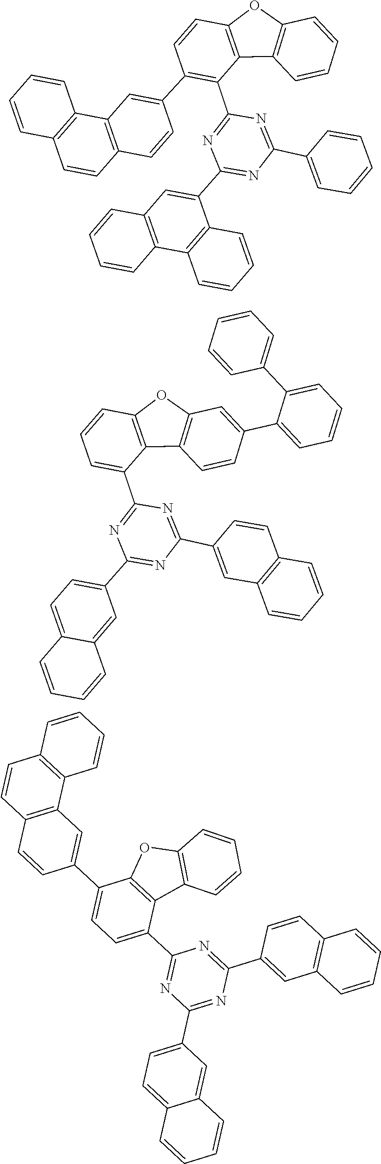

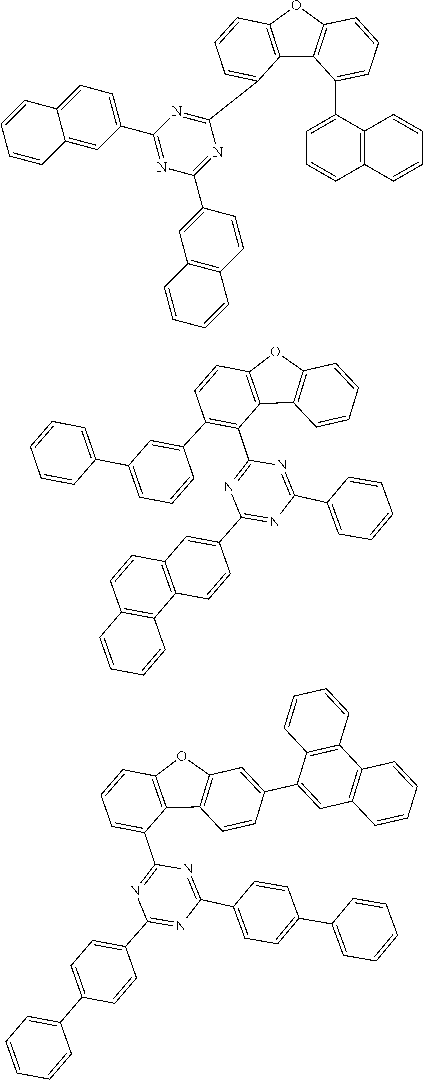

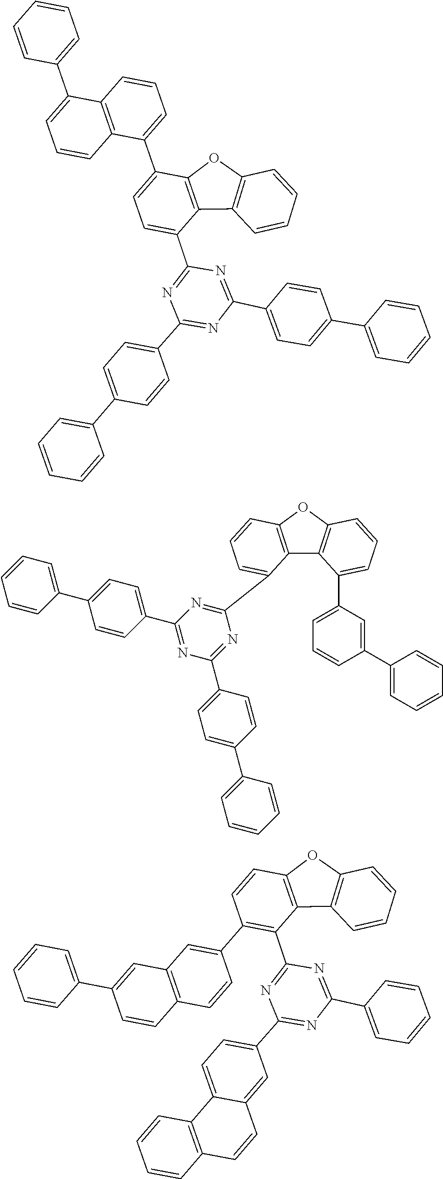

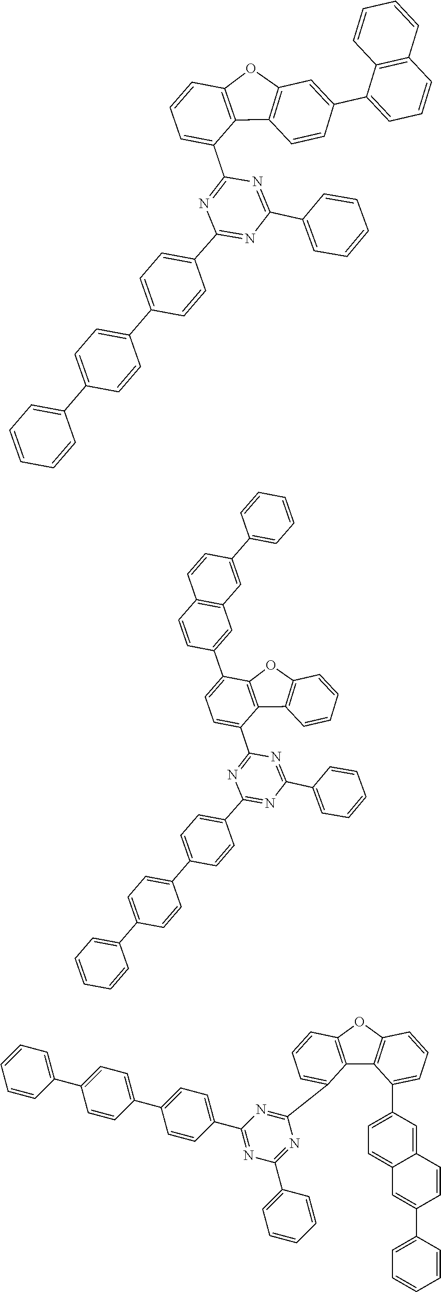 [SMILES: c1ccc(-c2ccc(-c3ccc(-c4nc(-c5ccccc5)nc(-c5cccc6oc7cc(-c8cccc9ccccc89)ccc7c56)n4)cc3)cc2)cc1 ; c1ccc(-c2ccc(-c3ccc(-c4nc(-c5ccccc5)nc(-c5ccc(-c6ccc7ccc(-c8ccccc8)cc7c6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9cc(-c%10ccccc%10)ccc9c8)c7c56)n4)cc3)cc2)cc1]

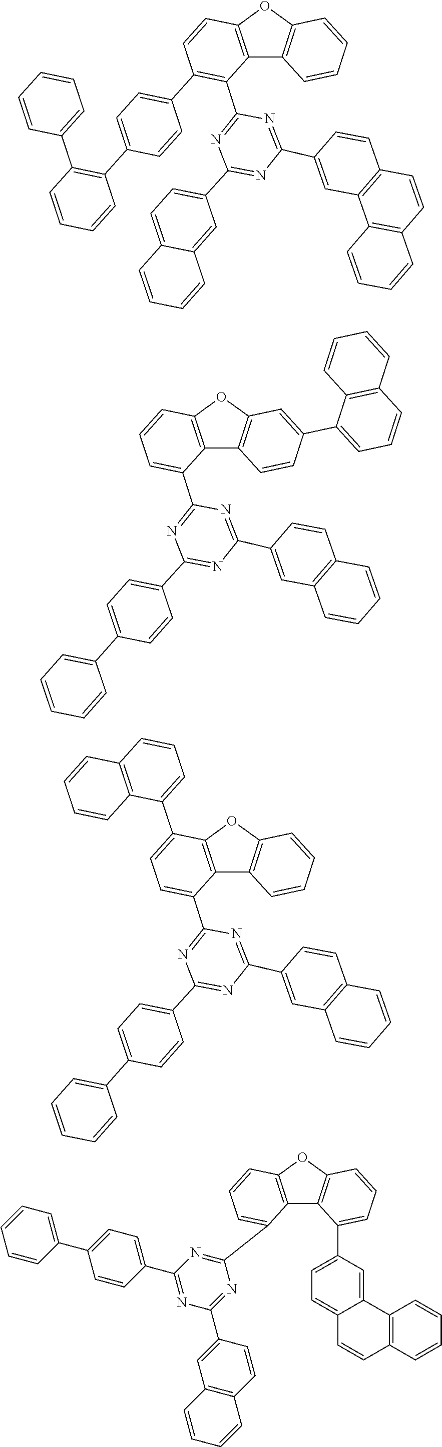

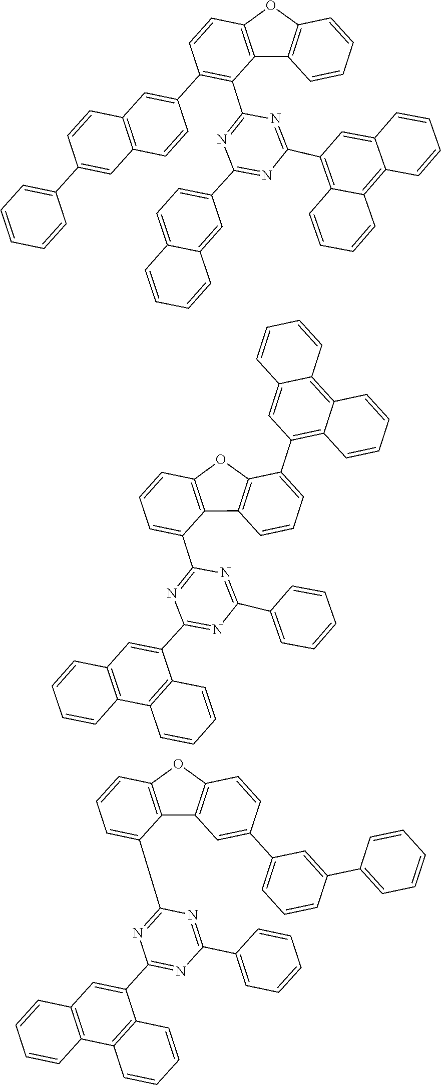

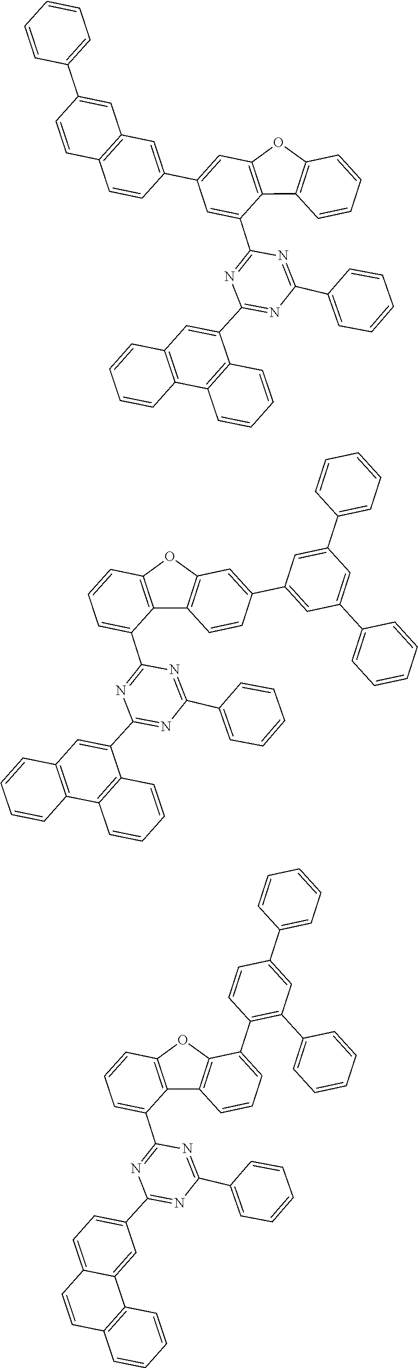 [SMILES: c1ccc(-c2ccc3ccc(-c4cc(-c5nc(-c6ccccc6)nc(-c6cc7ccccc7c7ccccc67)n5)c5c(c4)oc4ccccc45)cc3c2)cc1 ; c1ccc(-c2cc(-c3ccccc3)cc(-c3ccc4c(c3)oc3cccc(-c5nc(-c6ccccc6)nc(-c6cc7ccccc7c7ccccc67)n5)c34)c2)cc1 ; c1ccc(-c2ccc(-c3cccc4c3oc3cccc(-c5nc(-c6ccccc6)nc(-c6ccc7ccc8ccccc8c7c6)n5)c34)c(-c3ccccc3)c2)cc1]

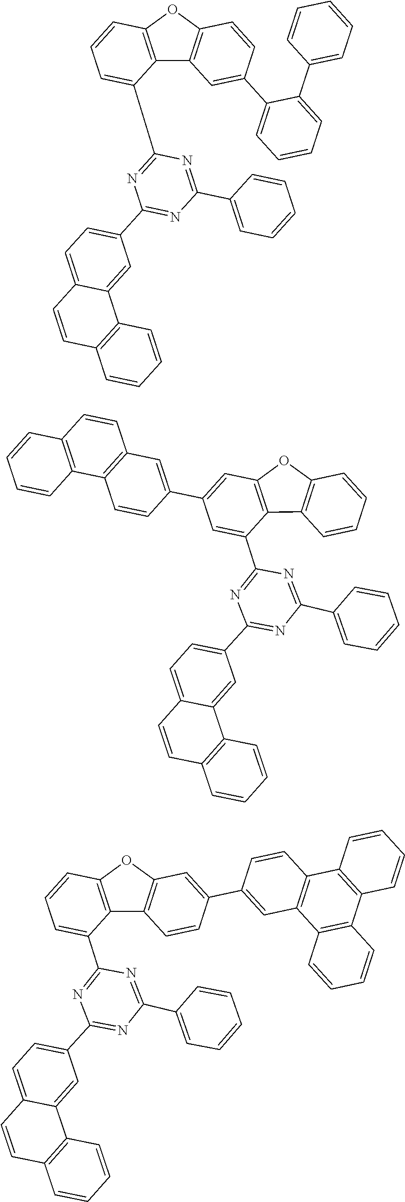

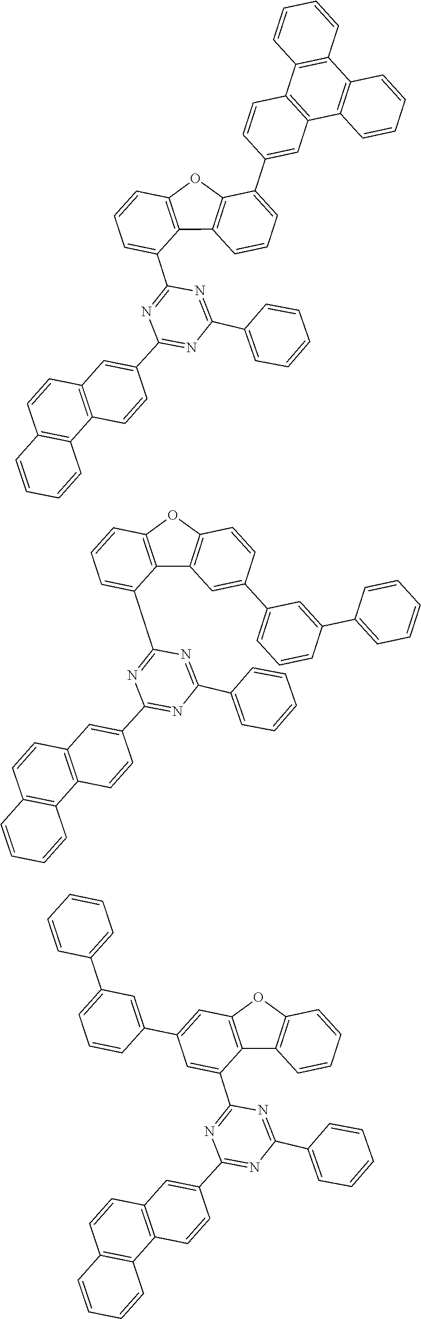

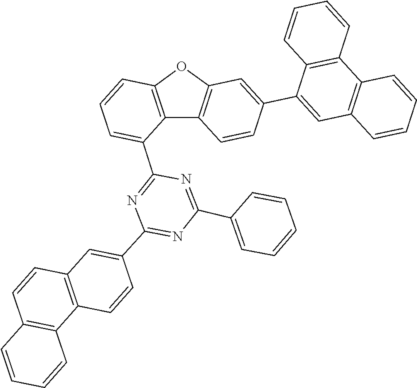 [SMILES: c1ccc(-c2nc(-c3ccc4c(ccc5ccccc54)c3)nc(-c3cccc4oc5cc(-c6cc7ccccc7c7ccccc67)ccc5c34)n2)cc1]

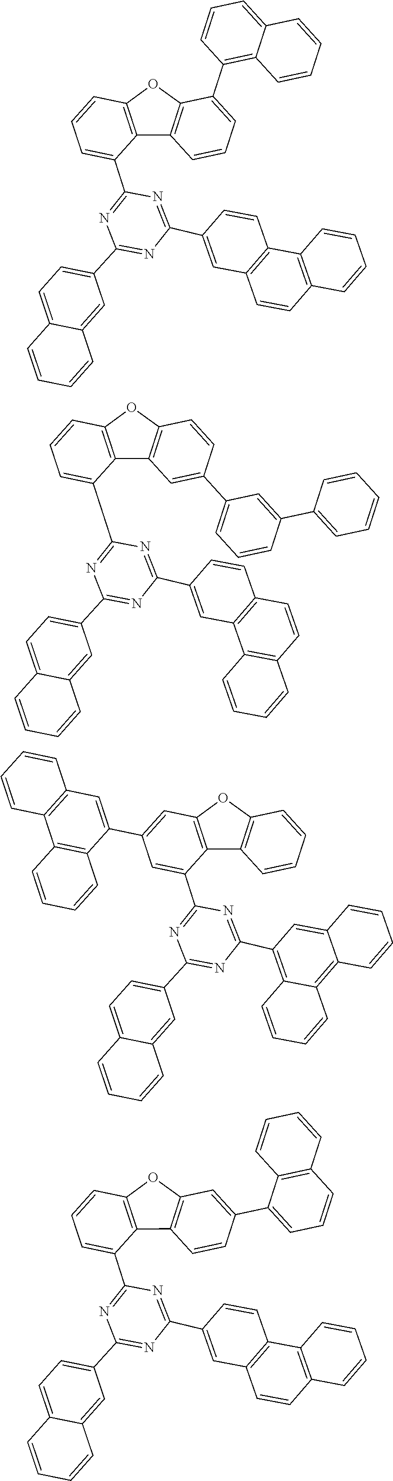

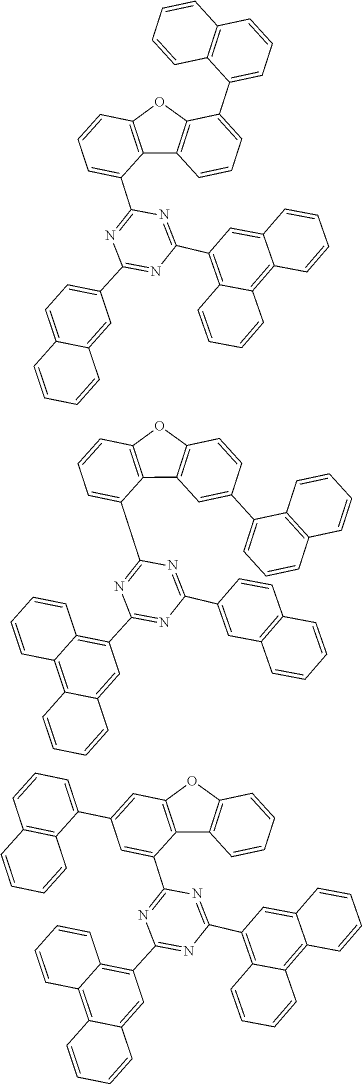

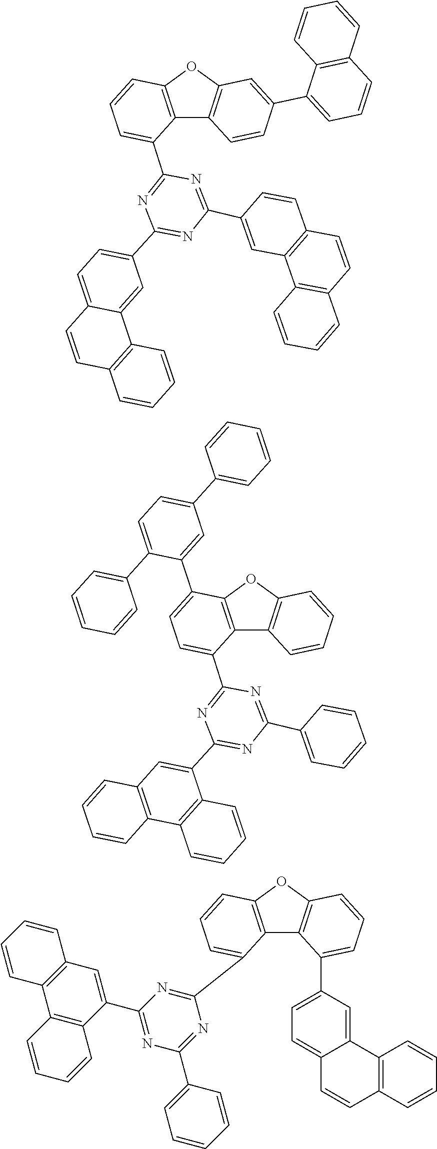

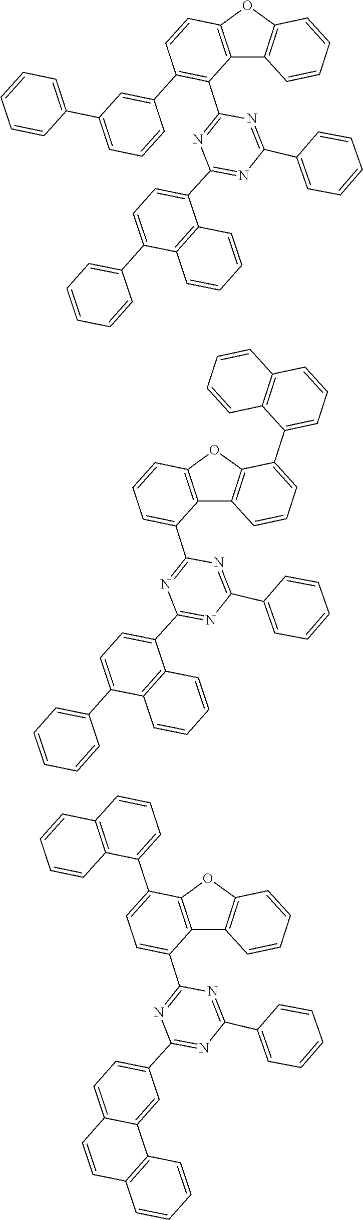

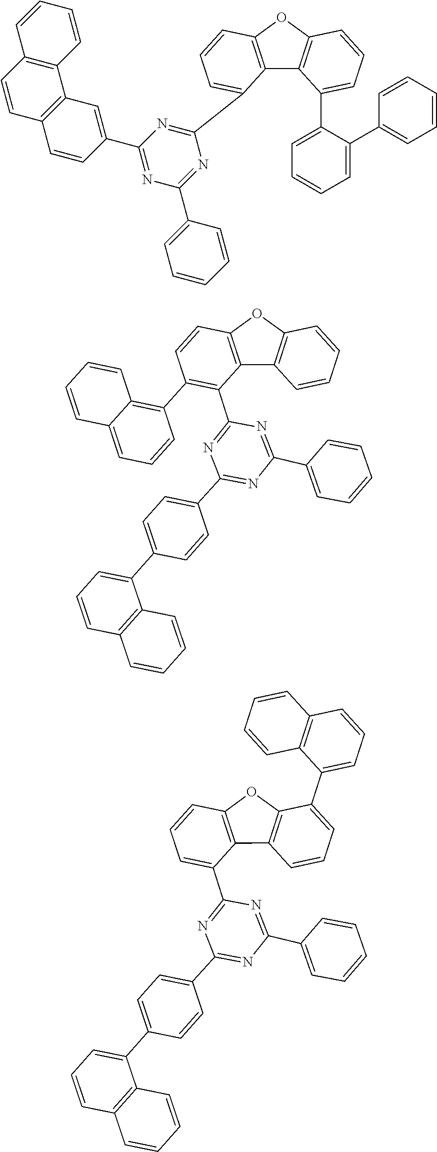

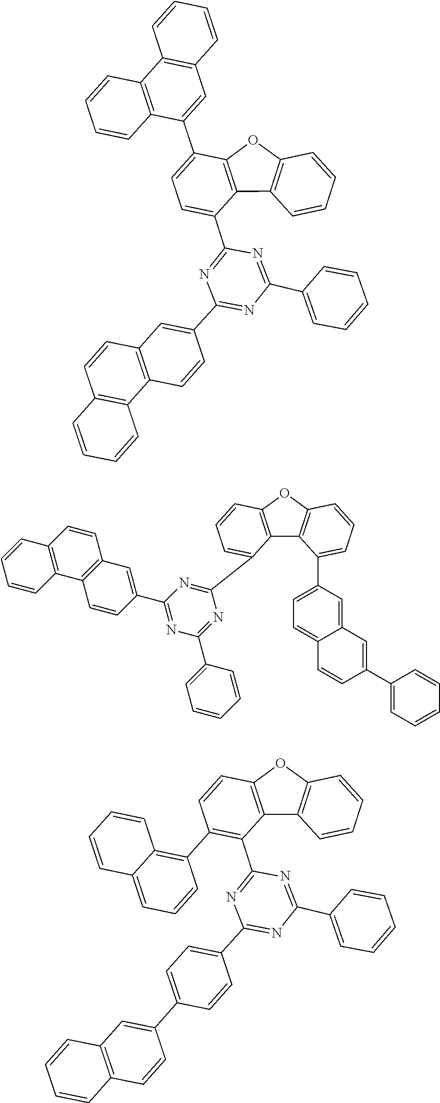 [SMILES: c1ccc(-c2nc(-c3ccc4c(ccc5ccccc54)c3)nc(-c3ccc(-c4cc5ccccc5c5ccccc45)c4oc5ccccc5c34)n2)cc1 ; c1ccc(-c2ccc3ccc(-c4cccc5oc6cccc(-c7nc(-c8ccccc8)nc(-c8ccc9c(ccc%10ccccc%109)c8)n7)c6c45)cc3c2)cc1 ; c1ccc(-c2nc(-c3ccc(-c4ccc5ccccc5c4)cc3)nc(-c3c(-c4cccc5ccccc45)ccc4oc5ccccc5c34)n2)cc1]

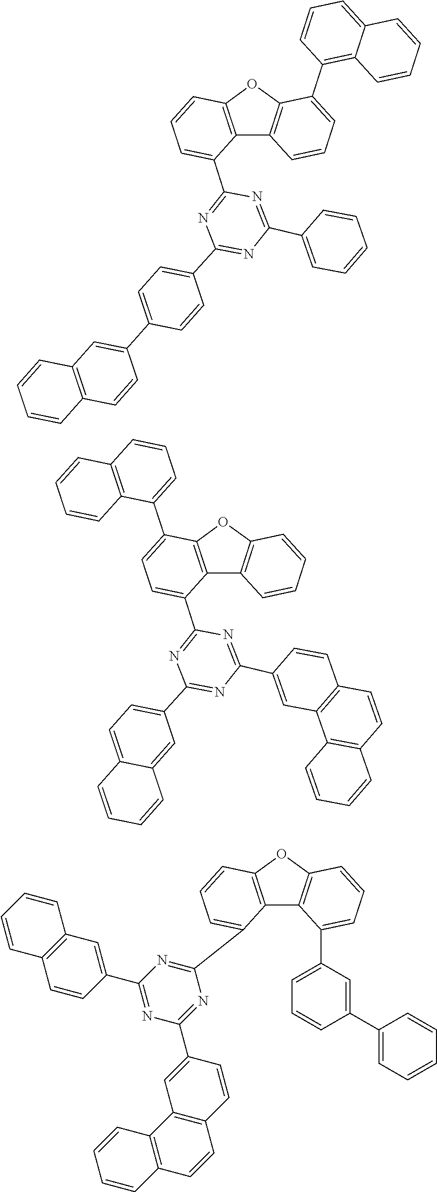

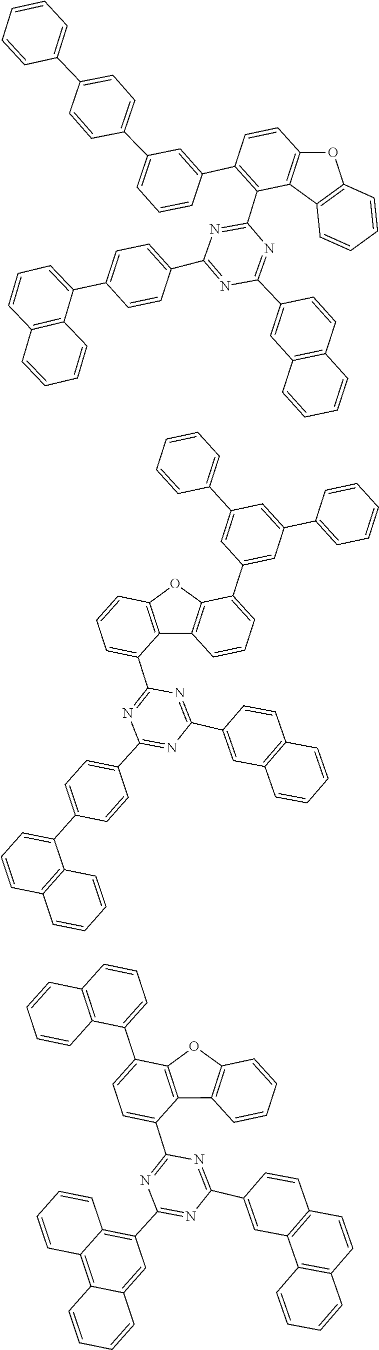

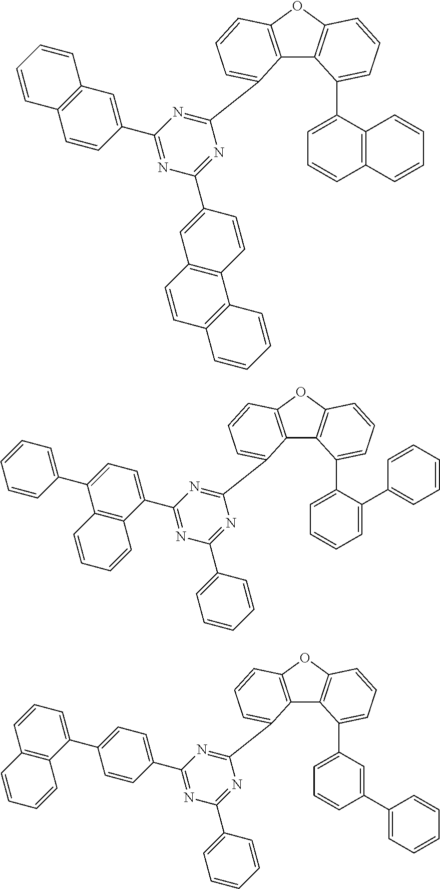

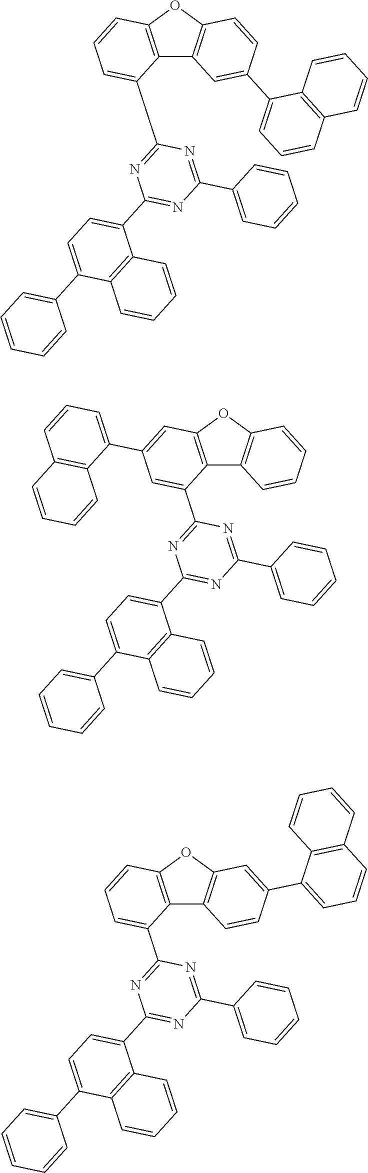 [SMILES: c1ccc(-c2nc(-c3ccc(-c4ccccc4)c4ccccc34)nc(-c3cccc4oc5ccc(-c6cccc7ccccc67)cc5c34)n2)cc1 ; c1ccc(-c2nc(-c3ccc(-c4ccccc4)c4ccccc34)nc(-c3cc(-c4cccc5ccccc45)cc4oc5ccccc5c34)n2)cc1 ; c1ccc(-c2nc(-c3ccc(-c4ccccc4)c4ccccc34)nc(-c3cccc4oc5cc(-c6cccc7ccccc67)ccc5c34)n2)cc1]

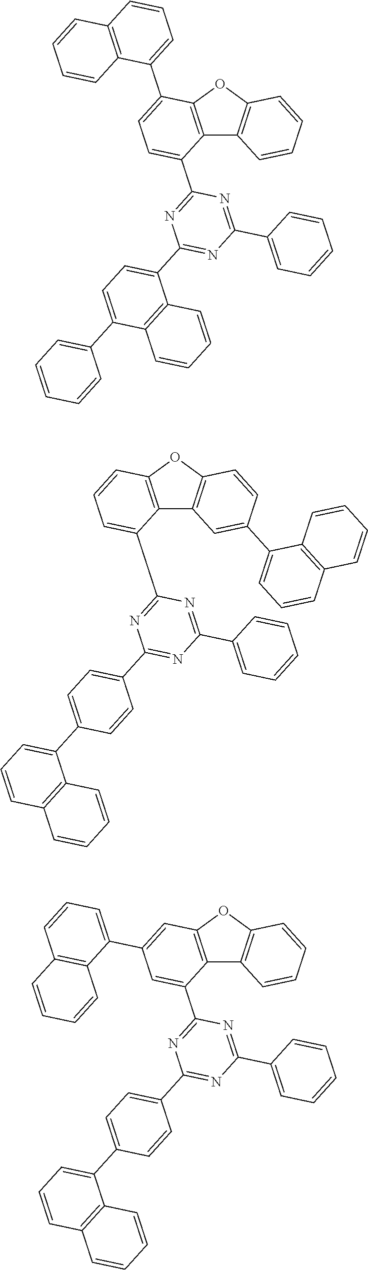 [SMILES: c1ccc(-c2nc(-c3ccc(-c4ccccc4)c4ccccc34)nc(-c3ccc(-c4cccc5ccccc45)c4oc5ccccc5c34)n2)cc1 ; c1ccc(-c2nc(-c3ccc(-c4cccc5ccccc45)cc3)nc(-c3cccc4oc5ccc(-c6cccc7ccccc67)cc5c34)n2)cc1 ; c1ccc(-c2nc(-c3ccc(-c4cccc5ccccc45)cc3)nc(-c3cc(-c4cccc5ccccc45)cc4oc5ccccc5c34)n2)cc1]

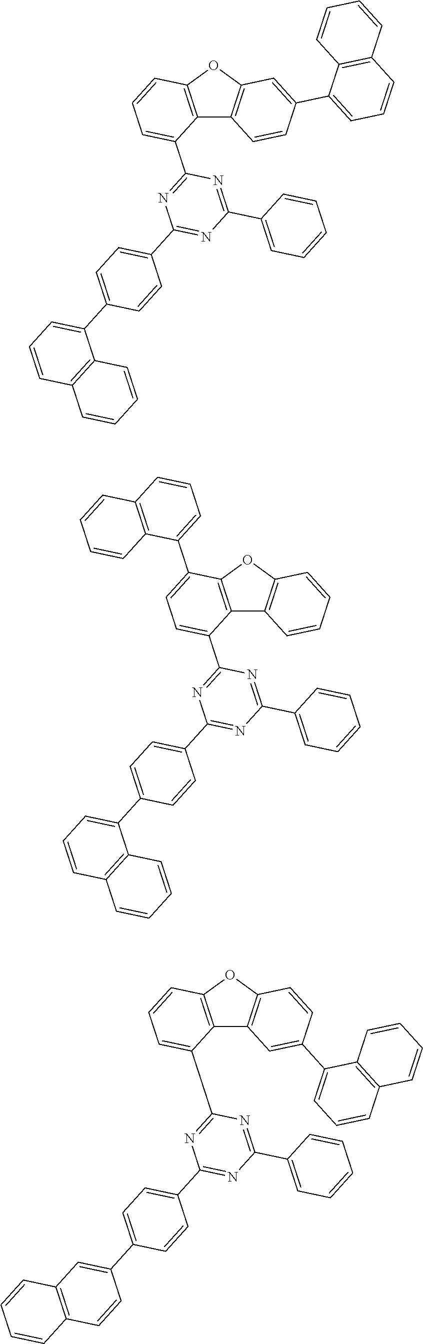 [SMILES: c1ccc(-c2nc(-c3ccc(-c4cccc5ccccc45)cc3)nc(-c3cccc4oc5cc(-c6cccc7ccccc67)ccc5c34)n2)cc1 ; c1ccc(-c2nc(-c3ccc(-c4cccc5ccccc45)cc3)nc(-c3ccc(-c4cccc5ccccc45)c4oc5ccccc5c34)n2)cc1 ; c1ccc(-c2nc(-c3ccc(-c4ccc5ccccc5c4)cc3)nc(-c3cccc4oc5ccc(-c6cccc7ccccc67)cc5c34)n2)cc1]

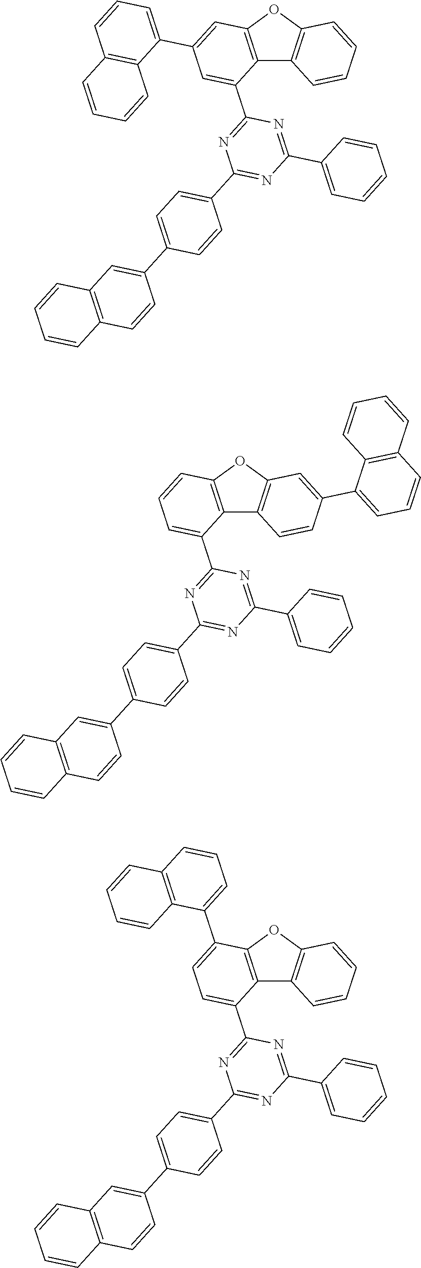 [SMILES: c1ccc(-c2nc(-c3ccc(-c4ccc5ccccc5c4)cc3)nc(-c3cc(-c4cccc5ccccc45)cc4oc5ccccc5c34)n2)cc1 ; c1ccc(-c2nc(-c3ccc(-c4ccc5ccccc5c4)cc3)nc(-c3cccc4oc5cc(-c6cccc7ccccc67)ccc5c34)n2)cc1 ; c1ccc(-c2nc(-c3ccc(-c4ccc5ccccc5c4)cc3)nc(-c3ccc(-c4cccc5ccccc45)c4oc5ccccc5c34)n2)cc1]

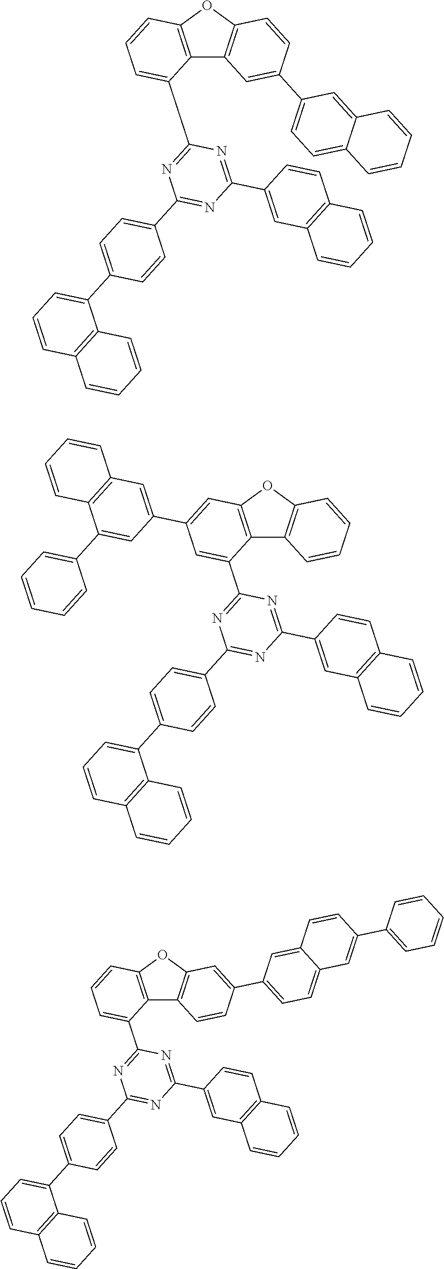 [SMILES: c1ccc2cc(-c3ccc4oc5cccc(-c6nc(-c7ccc(-c8cccc9ccccc89)cc7)nc(-c7ccc8ccccc8c7)n6)c5c4c3)ccc2c1 ; c1ccc(-c2cc(-c3cc(-c4nc(-c5ccc(-c6cccc7ccccc67)cc5)nc(-c5ccc6ccccc6c5)n4)c4c(c3)oc3ccccc34)cc3ccccc23)cc1 ; c1ccc(-c2ccc3cc(-c4ccc5c(c4)oc4cccc(-c6nc(-c7ccc(-c8cccc9ccccc89)cc7)nc(-c7ccc8ccccc8c7)n6)c45)ccc3c2)cc1]

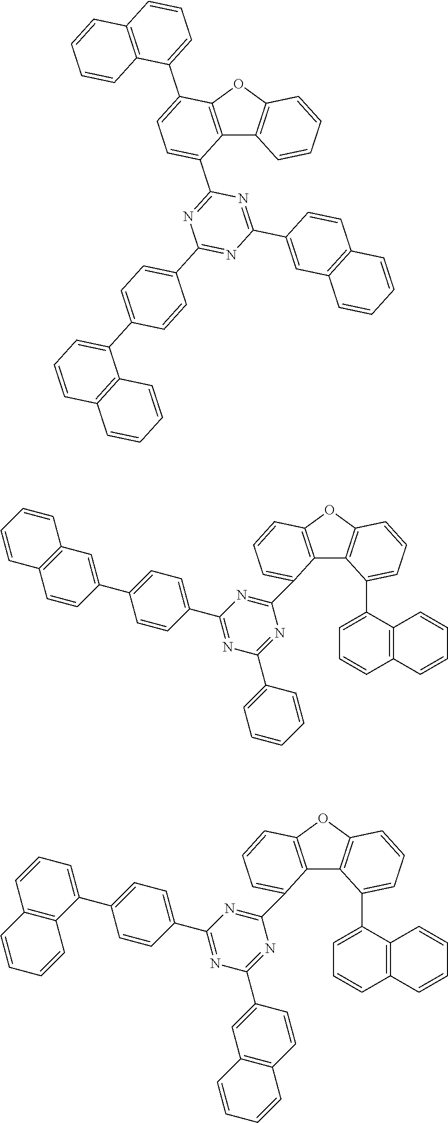 [SMILES: c1ccc2cc(-c3nc(-c4ccc(-c5cccc6ccccc56)cc4)nc(-c4ccc(-c5cccc6ccccc56)c5oc6ccccc6c45)n3)ccc2c1 ; c1ccc(-c2nc(-c3ccc(-c4ccc5ccccc5c4)cc3)nc(-c3cccc4oc5cccc(-c6cccc7ccccc67)c5c34)n2)cc1 ; c1ccc2cc(-c3nc(-c4ccc(-c5cccc6ccccc56)cc4)nc(-c4cccc5oc6cccc(-c7cccc8ccccc78)c6c45)n3)ccc2c1]

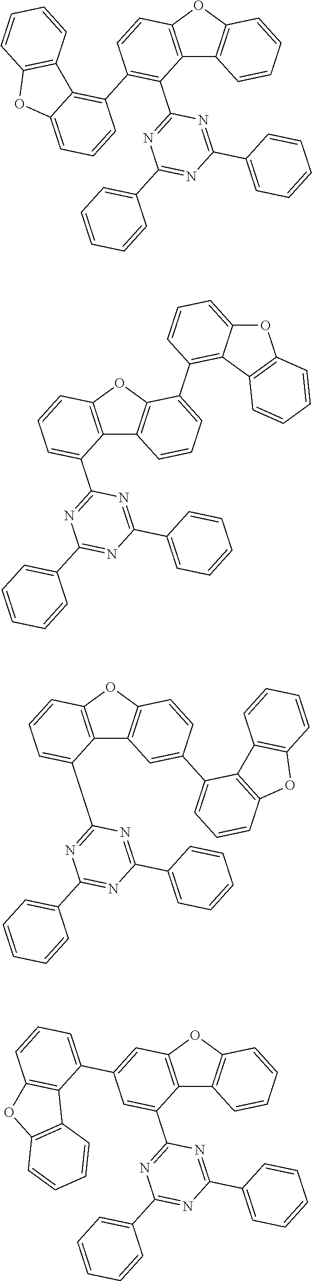 [SMILES: c1ccc(-c2nc(-c3ccccc3)nc(-c3c(-c4cccc5oc6ccccc6c45)ccc4oc5ccccc5c34)n2)cc1 ; c1ccc(-c2nc(-c3ccccc3)nc(-c3cccc4oc5c(-c6cccc7oc8ccccc8c67)cccc5c34)n2)cc1 ; c1ccc(-c2nc(-c3ccccc3)nc(-c3cccc4oc5ccc(-c6cccc7oc8ccccc8c67)cc5c34)n2)cc1 ; c1ccc(-c2nc(-c3ccccc3)nc(-c3cc(-c4cccc5oc6ccccc6c45)cc4oc5ccccc5c34)n2)cc1]

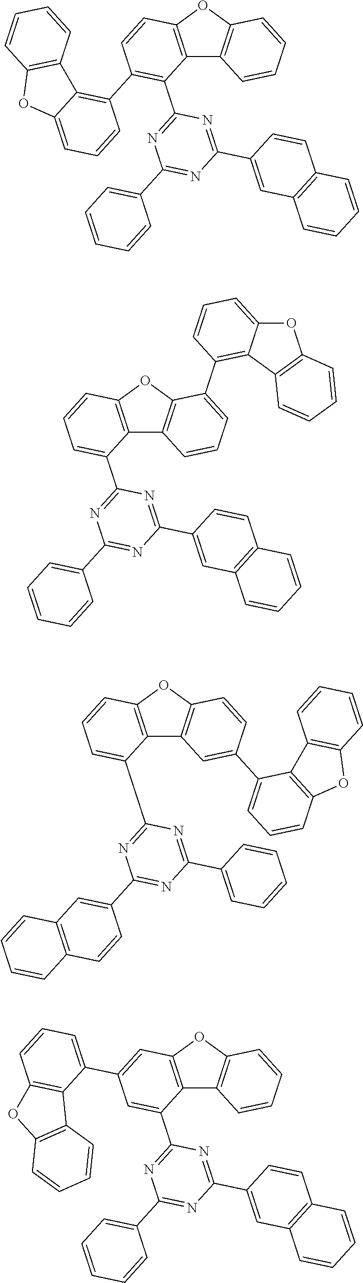 [SMILES: c1ccc(-c2nc(-c3ccc4ccccc4c3)nc(-c3c(-c4cccc5oc6ccccc6c45)ccc4oc5ccccc5c34)n2)cc1 ; c1ccc(-c2nc(-c3ccc4ccccc4c3)nc(-c3cccc4oc5c(-c6cccc7oc8ccccc8c67)cccc5c34)n2)cc1 ; c1ccc(-c2nc(-c3ccc4ccccc4c3)nc(-c3cccc4oc5ccc(-c6cccc7oc8ccccc8c67)cc5c34)n2)cc1 ; c1ccc(-c2nc(-c3ccc4ccccc4c3)nc(-c3cc(-c4cccc5oc6ccccc6c45)cc4oc5ccccc5c34)n2)cc1]

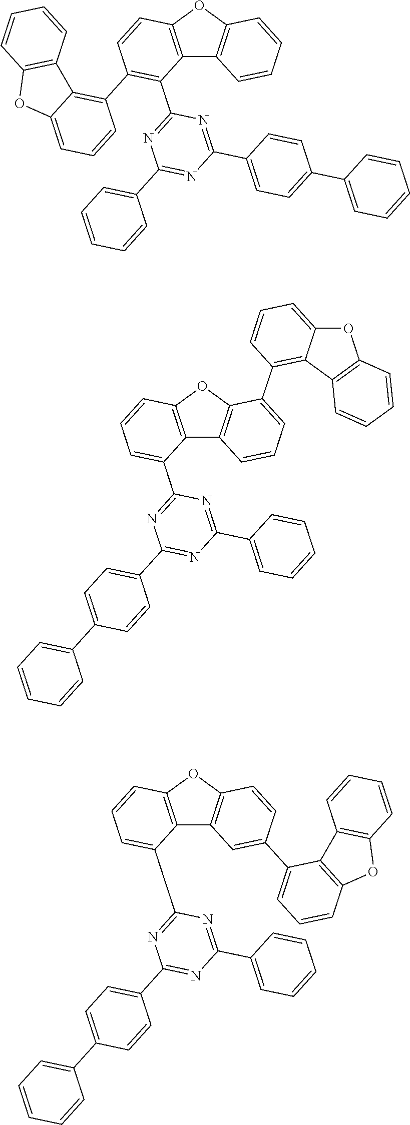 [SMILES: c1ccc(-c2ccc(-c3nc(-c4ccccc4)nc(-c4c(-c5cccc6oc7ccccc7c56)ccc5oc6ccccc6c45)n3)cc2)cc1 ; c1ccc(-c2ccc(-c3nc(-c4ccccc4)nc(-c4cccc5oc6c(-c7cccc8oc9ccccc9c78)cccc6c45)n3)cc2)cc1 ; c1ccc(-c2ccc(-c3nc(-c4ccccc4)nc(-c4cccc5oc6ccc(-c7cccc8oc9ccccc9c78)cc6c45)n3)cc2)cc1]

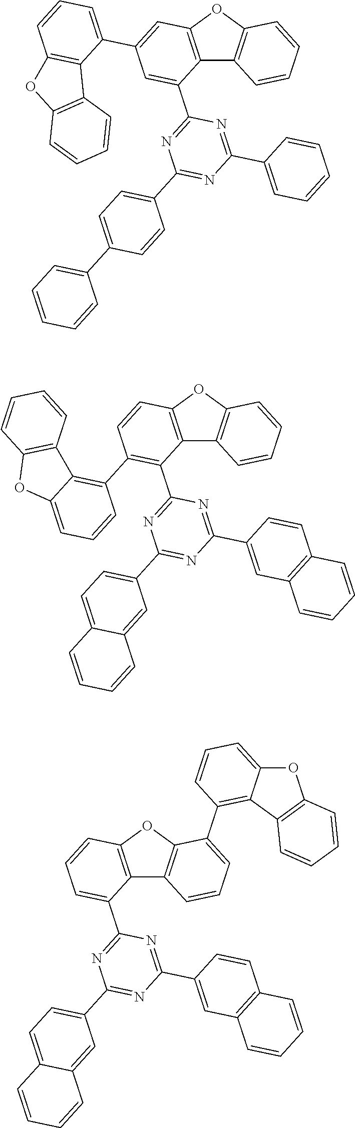 [SMILES: c1ccc(-c2ccc(-c3nc(-c4ccccc4)nc(-c4cc(-c5cccc6oc7ccccc7c56)cc5oc6ccccc6c45)n3)cc2)cc1 ; c1ccc2cc(-c3nc(-c4ccc5ccccc5c4)nc(-c4c(-c5cccc6oc7ccccc7c56)ccc5oc6ccccc6c45)n3)ccc2c1 ; c1ccc2cc(-c3nc(-c4ccc5ccccc5c4)nc(-c4cccc5oc6c(-c7cccc8oc9ccccc9c78)cccc6c45)n3)ccc2c1]

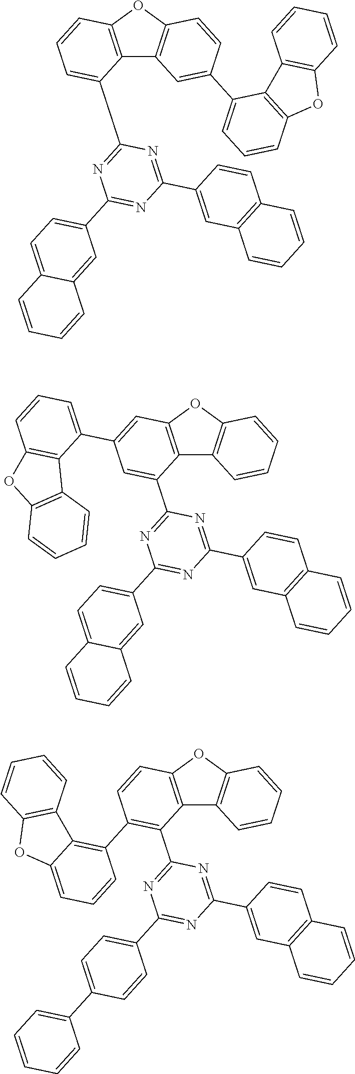 [SMILES: c1ccc2cc(-c3nc(-c4ccc5ccccc5c4)nc(-c4cccc5oc6ccc(-c7cccc8oc9ccccc9c78)cc6c45)n3)ccc2c1 ; c1ccc2cc(-c3nc(-c4ccc5ccccc5c4)nc(-c4cc(-c5cccc6oc7ccccc7c56)cc5oc6ccccc6c45)n3)ccc2c1 ; c1ccc(-c2ccc(-c3nc(-c4ccc5ccccc5c4)nc(-c4c(-c5cccc6oc7ccccc7c56)ccc5oc6ccccc6c45)n3)cc2)cc1]

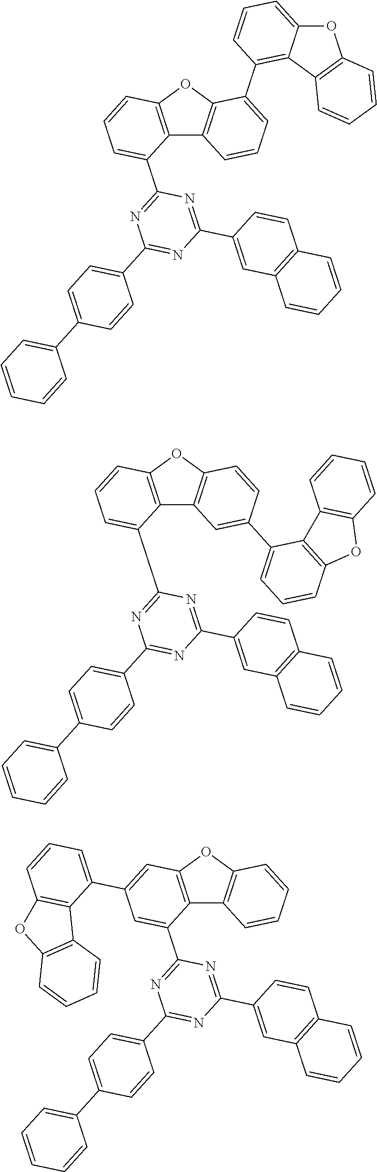

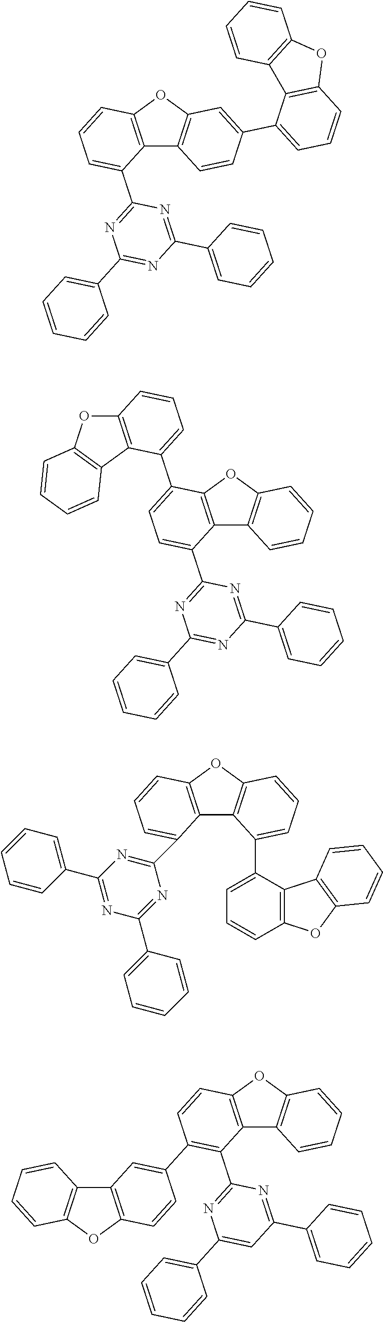 [SMILES: c1ccc(-c2nc(-c3ccccc3)nc(-c3cccc4oc5cc(-c6cccc7oc8ccccc8c67)ccc5c34)n2)cc1 ; c1ccc(-c2nc(-c3ccccc3)nc(-c3ccc(-c4cccc5oc6ccccc6c45)c4oc5ccccc5c34)n2)cc1 ; c1ccc(-c2nc(-c3ccccc3)nc(-c3cccc4oc5cccc(-c6cccc7oc8ccccc8c67)c5c34)n2)cc1 ; c1ccc(-c2cc(-c3ccccc3)nc(-c3c(-c4ccc5oc6ccccc6c5c4)ccc4oc5ccccc5c34)n2)cc1]

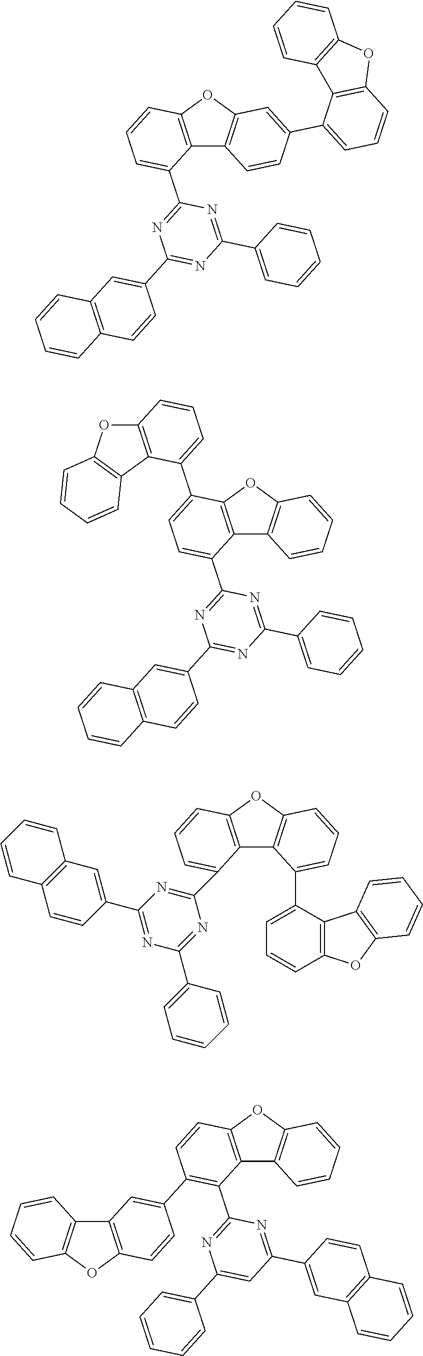 [SMILES: c1ccc(-c2nc(-c3ccc4ccccc4c3)nc(-c3cccc4oc5cc(-c6cccc7oc8ccccc8c67)ccc5c34)n2)cc1 ; c1ccc(-c2nc(-c3ccc4ccccc4c3)nc(-c3ccc(-c4cccc5oc6ccccc6c45)c4oc5ccccc5c34)n2)cc1 ; c1ccc(-c2nc(-c3ccc4ccccc4c3)nc(-c3cccc4oc5cccc(-c6cccc7oc8ccccc8c67)c5c34)n2)cc1 ; c1ccc(-c2cc(-c3ccc4ccccc4c3)nc(-c3c(-c4ccc5oc6ccccc6c5c4)ccc4oc5ccccc5c34)n2)cc1]

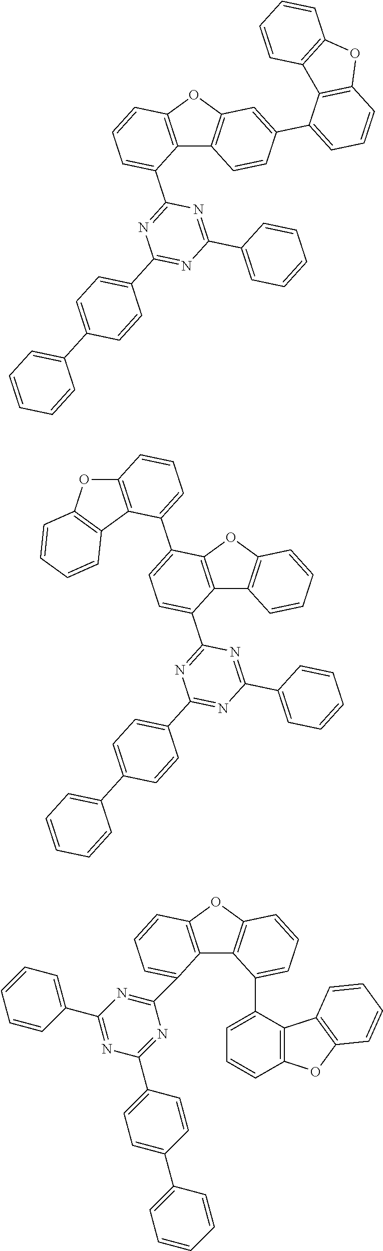 [SMILES: c1ccc(-c2ccc(-c3nc(-c4ccccc4)nc(-c4cccc5oc6cc(-c7cccc8oc9ccccc9c78)ccc6c45)n3)cc2)cc1 ; c1ccc(-c2ccc(-c3nc(-c4ccccc4)nc(-c4ccc(-c5cccc6oc7ccccc7c56)c5oc6ccccc6c45)n3)cc2)cc1 ; c1ccc(-c2ccc(-c3nc(-c4ccccc4)nc(-c4cccc5oc6cccc(-c7cccc8oc9ccccc9c78)c6c45)n3)cc2)cc1]

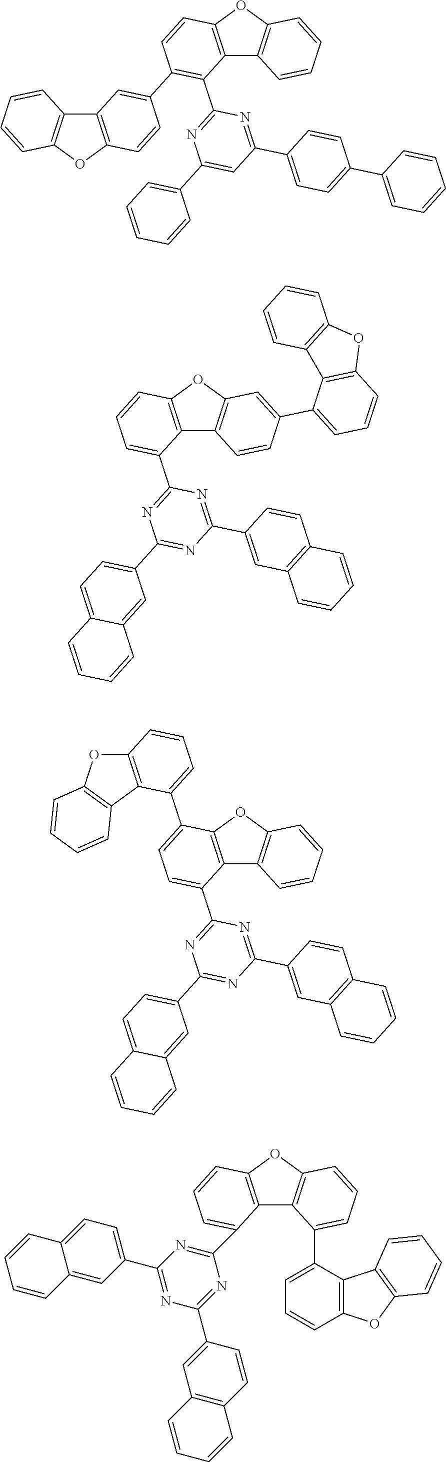 [SMILES: c1ccc(-c2ccc(-c3cc(-c4ccccc4)nc(-c4c(-c5ccc6oc7ccccc7c6c5)ccc5oc6ccccc6c45)n3)cc2)cc1 ; c1ccc2cc(-c3nc(-c4ccc5ccccc5c4)nc(-c4cccc5oc6cc(-c7cccc8oc9ccccc9c78)ccc6c45)n3)ccc2c1 ; c1ccc2cc(-c3nc(-c4ccc5ccccc5c4)nc(-c4ccc(-c5cccc6oc7ccccc7c56)c5oc6ccccc6c45)n3)ccc2c1 ; c1ccc2cc(-c3nc(-c4ccc5ccccc5c4)nc(-c4cccc5oc6cccc(-c7cccc8oc9ccccc9c78)c6c45)n3)ccc2c1]

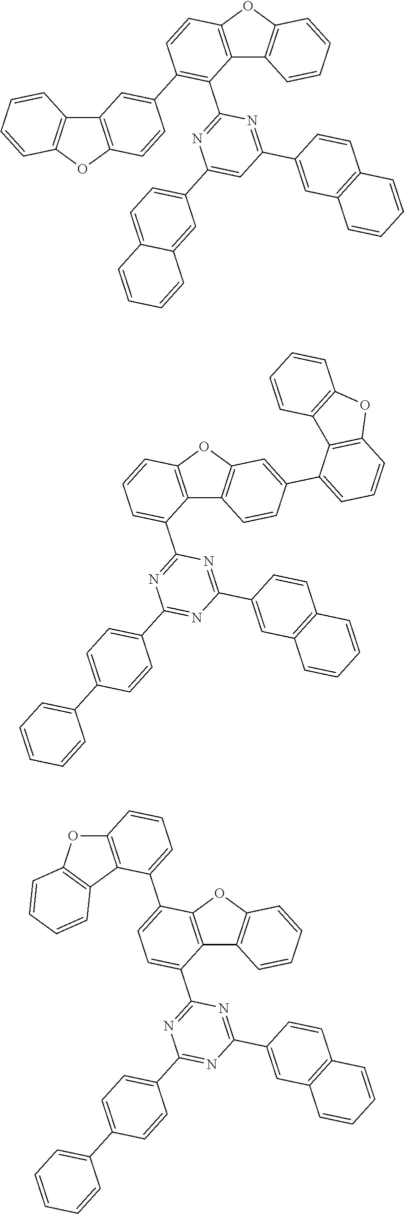 [SMILES: c1ccc2cc(-c3cc(-c4ccc5ccccc5c4)nc(-c4c(-c5ccc6oc7ccccc7c6c5)ccc5oc6ccccc6c45)n3)ccc2c1 ; c1ccc(-c2ccc(-c3nc(-c4ccc5ccccc5c4)nc(-c4cccc5oc6cc(-c7cccc8oc9ccccc9c78)ccc6c45)n3)cc2)cc1 ; c1ccc(-c2ccc(-c3nc(-c4ccc5ccccc5c4)nc(-c4ccc(-c5cccc6oc7ccccc7c56)c5oc6ccccc6c45)n3)cc2)cc1]

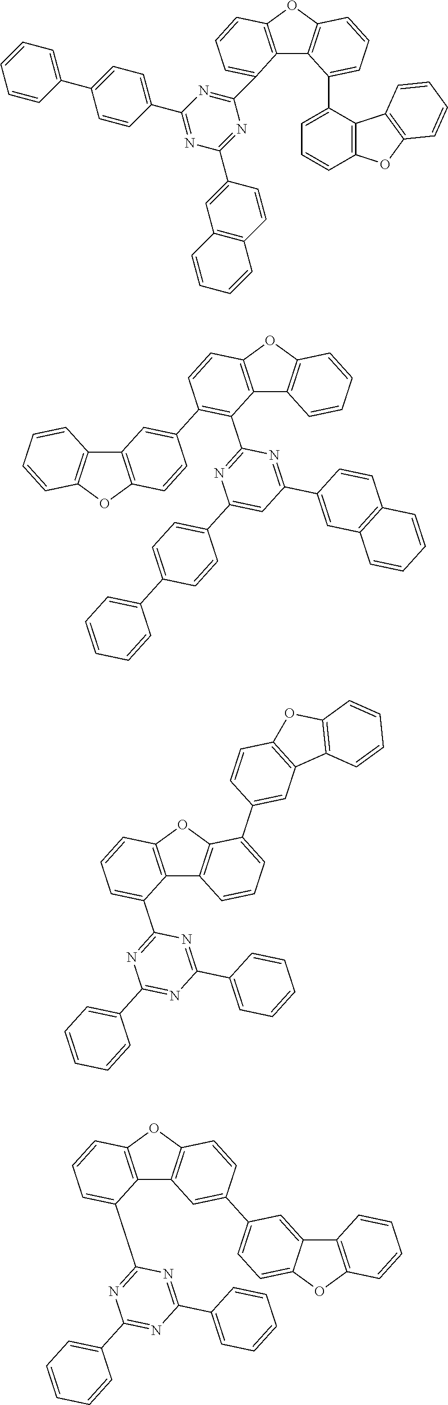 [SMILES: c1ccc(-c2ccc(-c3nc(-c4ccc5ccccc5c4)nc(-c4cccc5oc6cccc(-c7cccc8oc9ccccc9c78)c6c45)n3)cc2)cc1 ; c1ccc(-c2ccc(-c3cc(-c4ccc5ccccc5c4)nc(-c4c(-c5ccc6oc7ccccc7c6c5)ccc5oc6ccccc6c45)n3)cc2)cc1 ; c1ccc(-c2nc(-c3ccccc3)nc(-c3cccc4oc5c(-c6ccc7oc8ccccc8c7c6)cccc5c34)n2)cc1 ; c1ccc(-c2nc(-c3ccccc3)nc(-c3cccc4oc5ccc(-c6ccc7oc8ccccc8c7c6)cc5c34)n2)cc1]

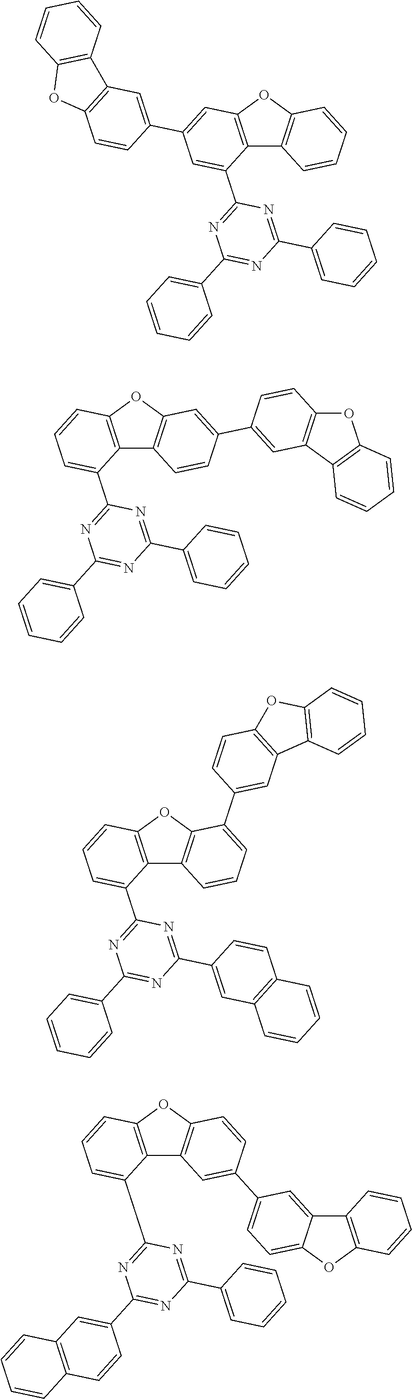 [SMILES: c1ccc(-c2nc(-c3ccccc3)nc(-c3cc(-c4ccc5oc6ccccc6c5c4)cc4oc5ccccc5c34)n2)cc1 ; c1ccc(-c2nc(-c3ccccc3)nc(-c3cccc4oc5cc(-c6ccc7oc8ccccc8c7c6)ccc5c34)n2)cc1 ; c1ccc(-c2nc(-c3ccc4ccccc4c3)nc(-c3cccc4oc5c(-c6ccc7oc8ccccc8c7c6)cccc5c34)n2)cc1 ; c1ccc(-c2nc(-c3ccc4ccccc4c3)nc(-c3cccc4oc5ccc(-c6ccc7oc8ccccc8c7c6)cc5c34)n2)cc1]

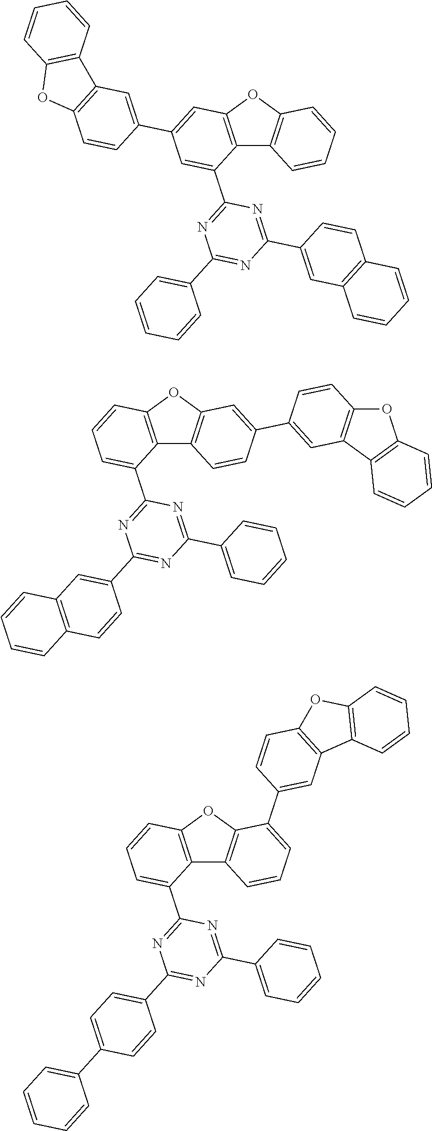 [SMILES: c1ccc(-c2nc(-c3ccc4ccccc4c3)nc(-c3cc(-c4ccc5oc6ccccc6c5c4)cc4oc5ccccc5c34)n2)cc1 ; c1ccc(-c2nc(-c3ccc4ccccc4c3)nc(-c3cccc4oc5cc(-c6ccc7oc8ccccc8c7c6)ccc5c34)n2)cc1 ; c1ccc(-c2ccc(-c3nc(-c4ccccc4)nc(-c4cccc5oc6c(-c7ccc8oc9ccccc9c8c7)cccc6c45)n3)cc2)cc1]

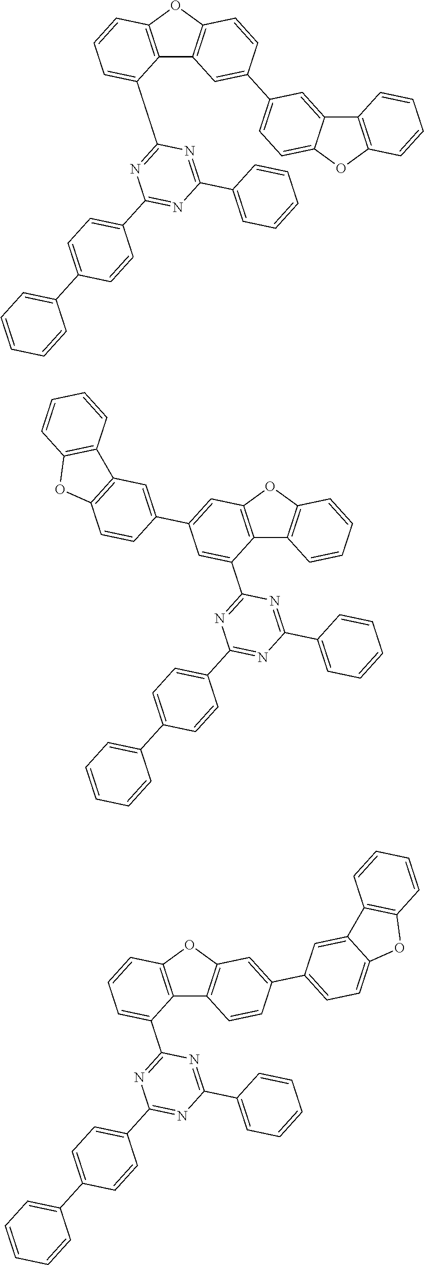

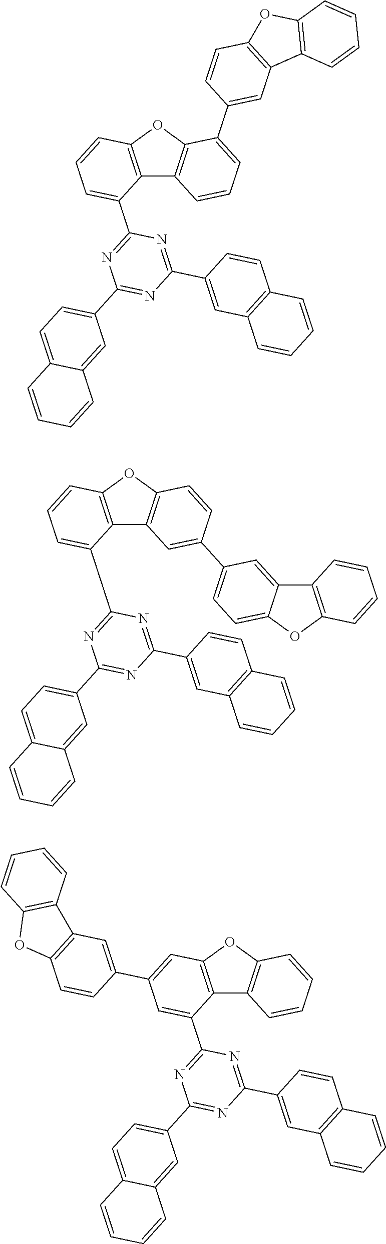

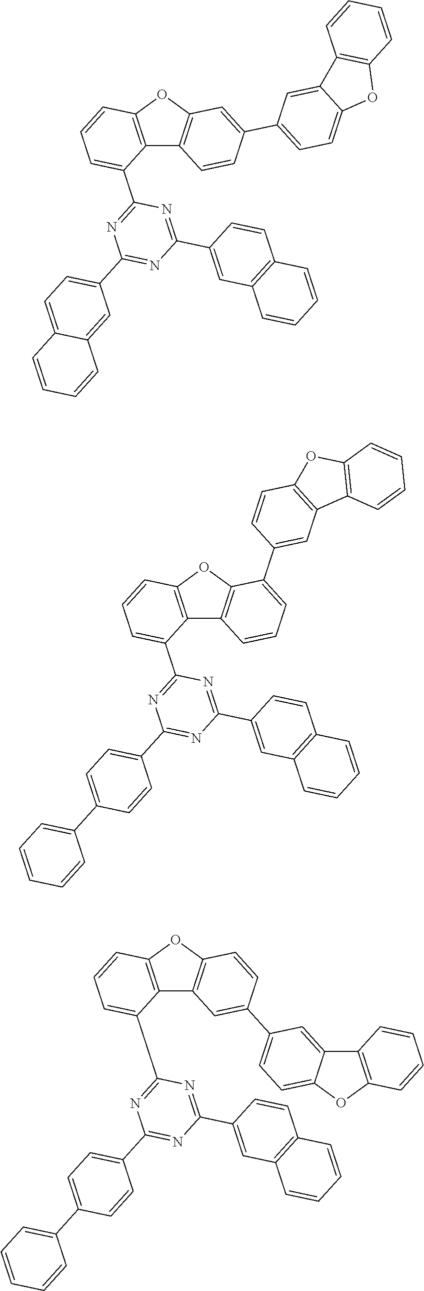 [SMILES: c1ccc2cc(-c3nc(-c4ccc5ccccc5c4)nc(-c4cccc5oc6cc(-c7ccc8oc9ccccc9c8c7)ccc6c45)n3)ccc2c1 ; c1ccc(-c2ccc(-c3nc(-c4ccc5ccccc5c4)nc(-c4cccc5oc6c(-c7ccc8oc9ccccc9c8c7)cccc6c45)n3)cc2)cc1 ; c1ccc(-c2ccc(-c3nc(-c4ccc5ccccc5c4)nc(-c4cccc5oc6ccc(-c7ccc8oc9ccccc9c8c7)cc6c45)n3)cc2)cc1]

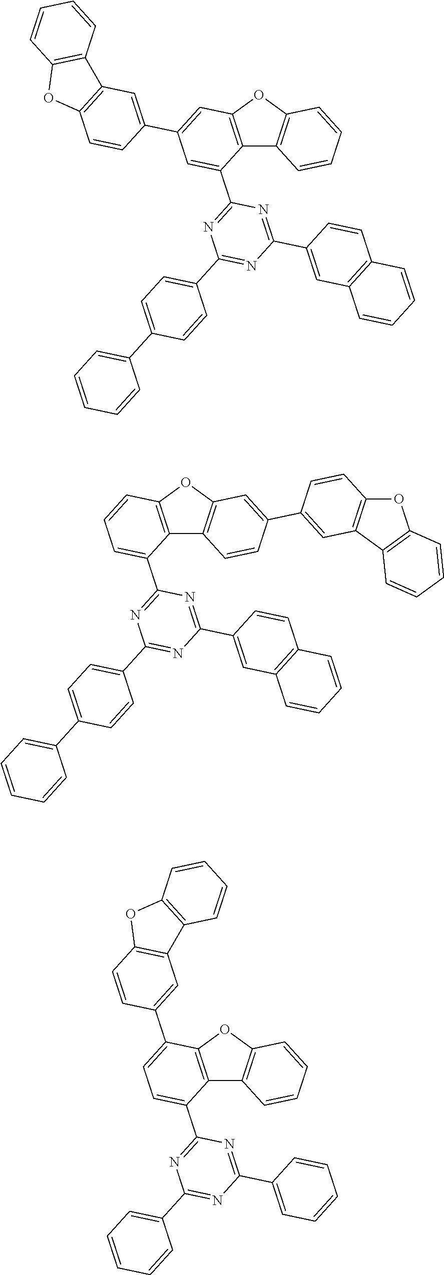 [SMILES: c1ccc(-c2ccc(-c3nc(-c4ccc5ccccc5c4)nc(-c4cc(-c5ccc6oc7ccccc7c6c5)cc5oc6ccccc6c45)n3)cc2)cc1 ; c1ccc(-c2ccc(-c3nc(-c4ccc5ccccc5c4)nc(-c4cccc5oc6cc(-c7ccc8oc9ccccc9c8c7)ccc6c45)n3)cc2)cc1 ; c1ccc(-c2nc(-c3ccccc3)nc(-c3ccc(-c4ccc5oc6ccccc6c5c4)c4oc5ccccc5c34)n2)cc1]

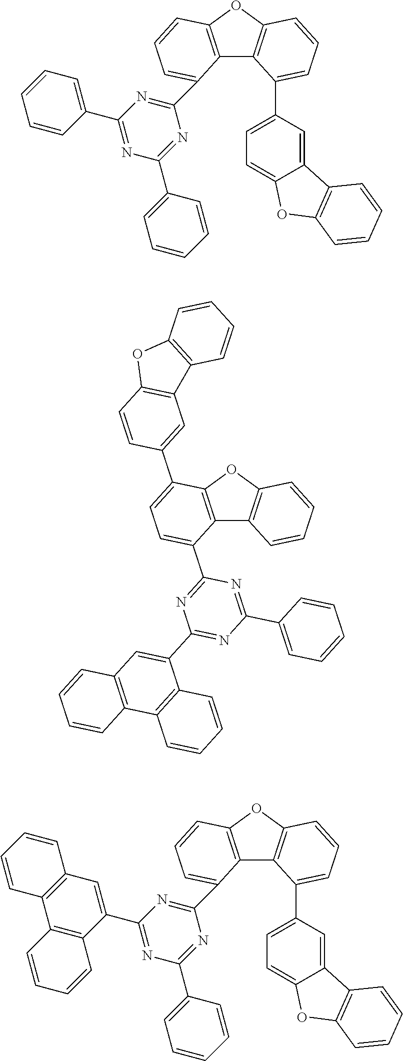 [SMILES: c1ccc(-c2nc(-c3ccccc3)nc(-c3cccc4oc5cccc(-c6ccc7oc8ccccc8c7c6)c5c34)n2)cc1 ; c1ccc(-c2nc(-c3cc4ccccc4c4ccccc34)nc(-c3ccc(-c4ccc5oc6ccccc6c5c4)c4oc5ccccc5c34)n2)cc1 ; c1ccc(-c2nc(-c3cc4ccccc4c4ccccc34)nc(-c3cccc4oc5cccc(-c6ccc7oc8ccccc8c7c6)c5c34)n2)cc1]

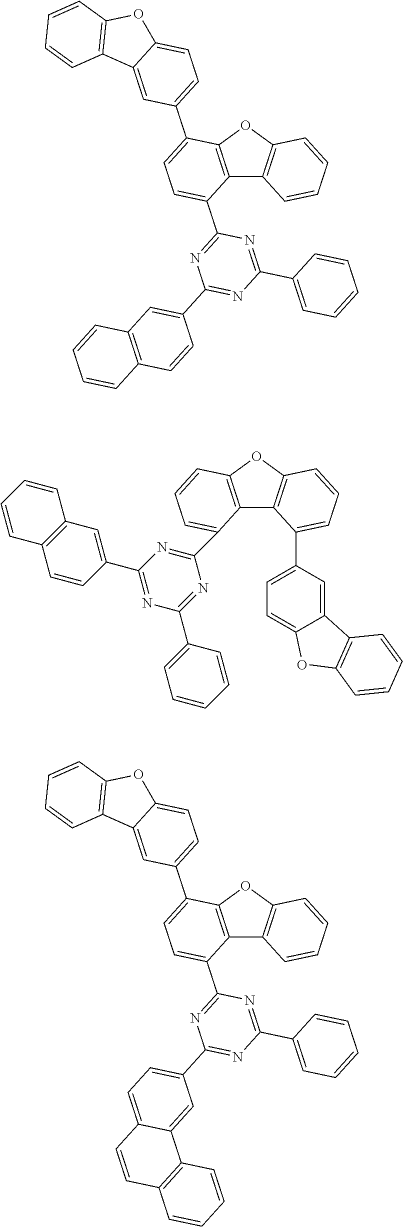 [SMILES: c1ccc(-c2nc(-c3ccc4ccccc4c3)nc(-c3ccc(-c4ccc5oc6ccccc6c5c4)c4oc5ccccc5c34)n2)cc1 ; c1ccc(-c2nc(-c3ccc4ccccc4c3)nc(-c3cccc4oc5cccc(-c6ccc7oc8ccccc8c7c6)c5c34)n2)cc1 ; c1ccc(-c2nc(-c3ccc4ccc5ccccc5c4c3)nc(-c3ccc(-c4ccc5oc6ccccc6c5c4)c4oc5ccccc5c34)n2)cc1]

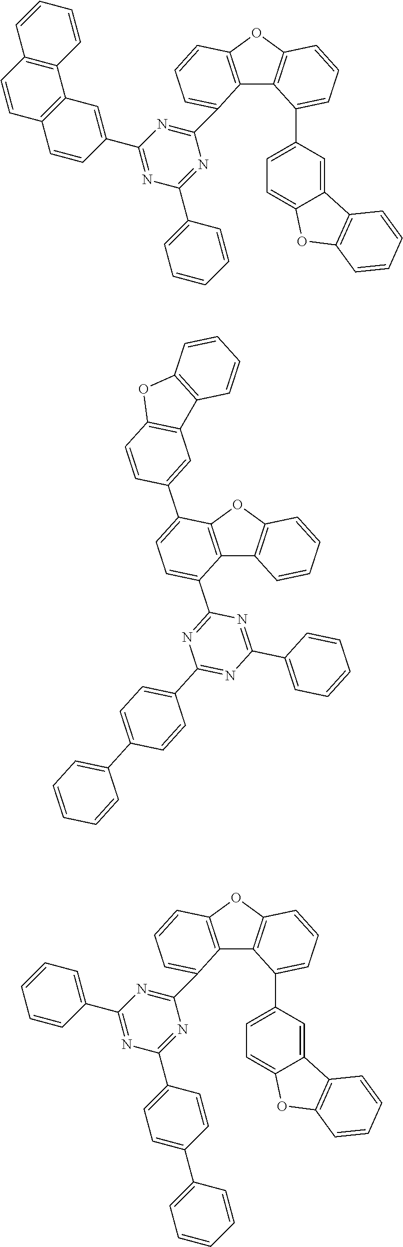 [SMILES: c1ccc(-c2nc(-c3ccc4ccc5ccccc5c4c3)nc(-c3cccc4oc5cccc(-c6ccc7oc8ccccc8c7c6)c5c34)n2)cc1 ; c1ccc(-c2ccc(-c3nc(-c4ccccc4)nc(-c4ccc(-c5ccc6oc7ccccc7c6c5)c5oc6ccccc6c45)n3)cc2)cc1 ; c1ccc(-c2ccc(-c3nc(-c4ccccc4)nc(-c4cccc5oc6cccc(-c7ccc8oc9ccccc9c8c7)c6c45)n3)cc2)cc1]

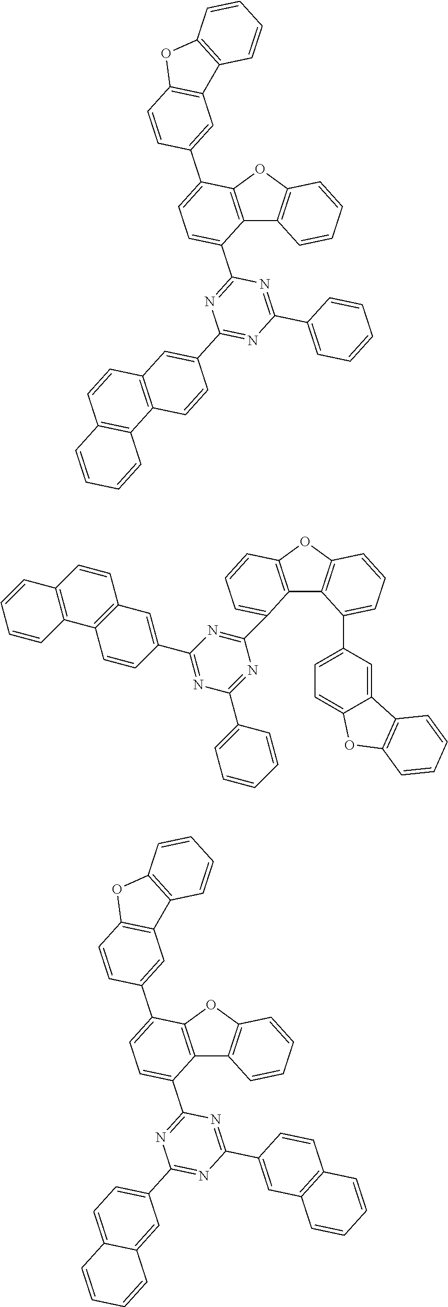 [SMILES: c1ccc(-c2nc(-c3ccc4c(ccc5ccccc54)c3)nc(-c3ccc(-c4ccc5oc6ccccc6c5c4)c4oc5ccccc5c34)n2)cc1 ; c1ccc(-c2nc(-c3ccc4c(ccc5ccccc54)c3)nc(-c3cccc4oc5cccc(-c6ccc7oc8ccccc8c7c6)c5c34)n2)cc1 ; c1ccc2cc(-c3nc(-c4ccc5ccccc5c4)nc(-c4ccc(-c5ccc6oc7ccccc7c6c5)c5oc6ccccc6c45)n3)ccc2c1]

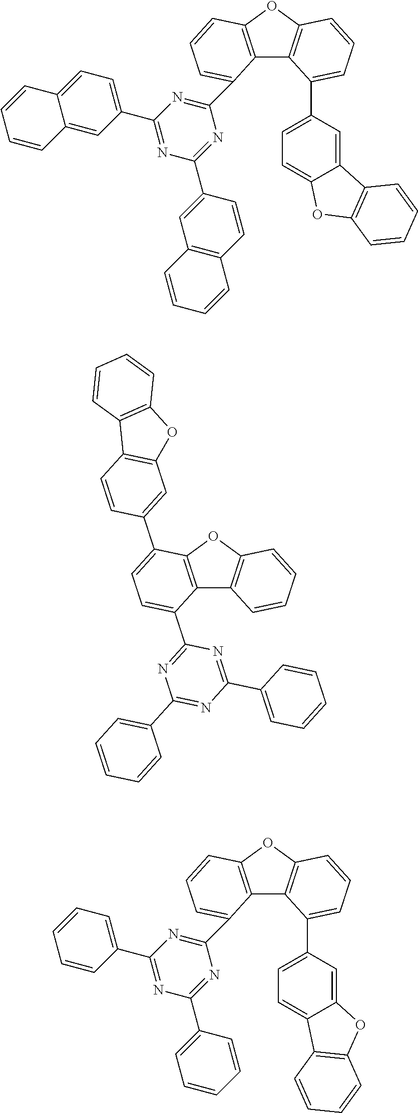 [SMILES: c1ccc2cc(-c3nc(-c4ccc5ccccc5c4)nc(-c4cccc5oc6cccc(-c7ccc8oc9ccccc9c8c7)c6c45)n3)ccc2c1 ; c1ccc(-c2nc(-c3ccccc3)nc(-c3ccc(-c4ccc5c(c4)oc4ccccc45)c4oc5ccccc5c34)n2)cc1 ; c1ccc(-c2nc(-c3ccccc3)nc(-c3cccc4oc5cccc(-c6ccc7c(c6)oc6ccccc67)c5c34)n2)cc1]

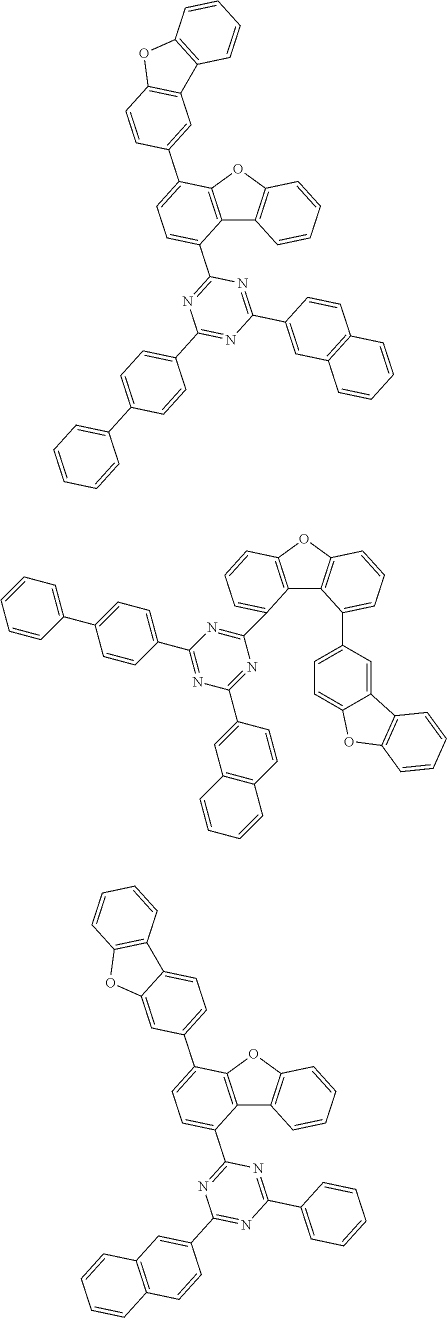 [SMILES: c1ccc(-c2ccc(-c3nc(-c4ccc5ccccc5c4)nc(-c4ccc(-c5ccc6oc7ccccc7c6c5)c5oc6ccccc6c45)n3)cc2)cc1 ; c1ccc(-c2ccc(-c3nc(-c4ccc5ccccc5c4)nc(-c4cccc5oc6cccc(-c7ccc8oc9ccccc9c8c7)c6c45)n3)cc2)cc1 ; c1ccc(-c2nc(-c3ccc4ccccc4c3)nc(-c3ccc(-c4ccc5c(c4)oc4ccccc45)c4oc5ccccc5c34)n2)cc1]

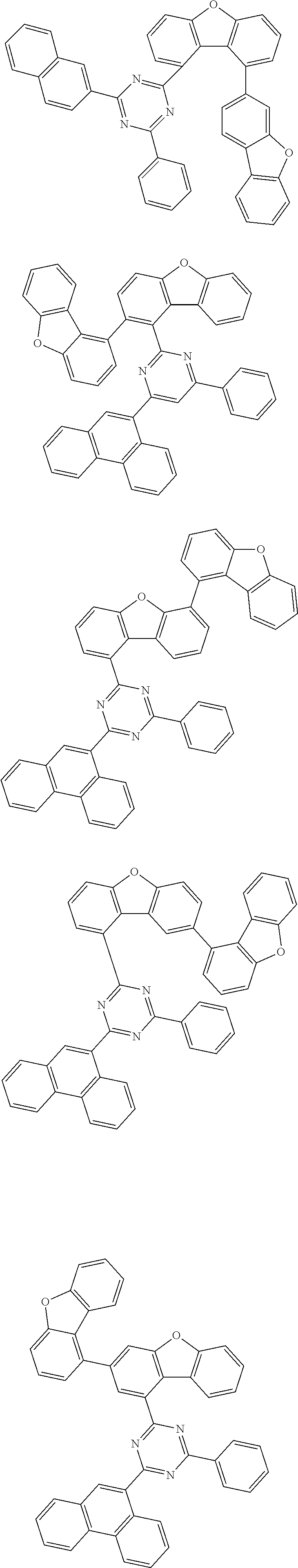 [SMILES: c1ccc(-c2nc(-c3ccc4ccccc4c3)nc(-c3cccc4oc5cccc(-c6ccc7c(c6)oc6ccccc67)c5c34)n2)cc1 ; c1ccc(-c2cc(-c3cc4ccccc4c4ccccc34)nc(-c3c(-c4cccc5oc6ccccc6c45)ccc4oc5ccccc5c34)n2)cc1 ; c1ccc(-c2nc(-c3cc4ccccc4c4ccccc34)nc(-c3cccc4oc5c(-c6cccc7oc8ccccc8c67)cccc5c34)n2)cc1 ; c1ccc(-c2nc(-c3cc4ccccc4c4ccccc34)nc(-c3cccc4oc5ccc(-c6cccc7oc8ccccc8c67)cc5c34)n2)cc1 ; c1ccc(-c2nc(-c3cc4ccccc4c4ccccc34)nc(-c3cc(-c4cccc5oc6ccccc6c45)cc4oc5ccccc5c34)n2)cc1]

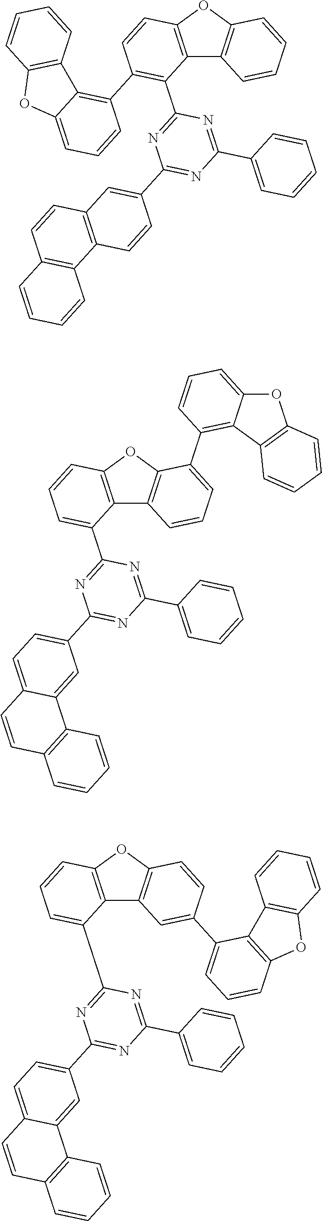 [SMILES: c1ccc(-c2nc(-c3ccc4c(ccc5ccccc54)c3)nc(-c3c(-c4cccc5oc6ccccc6c45)ccc4oc5ccccc5c34)n2)cc1 ; c1ccc(-c2nc(-c3ccc4ccc5ccccc5c4c3)nc(-c3cccc4oc5c(-c6cccc7oc8ccccc8c67)cccc5c34)n2)cc1 ; c1ccc(-c2nc(-c3ccc4ccc5ccccc5c4c3)nc(-c3cccc4oc5ccc(-c6cccc7oc8ccccc8c67)cc5c34)n2)cc1]

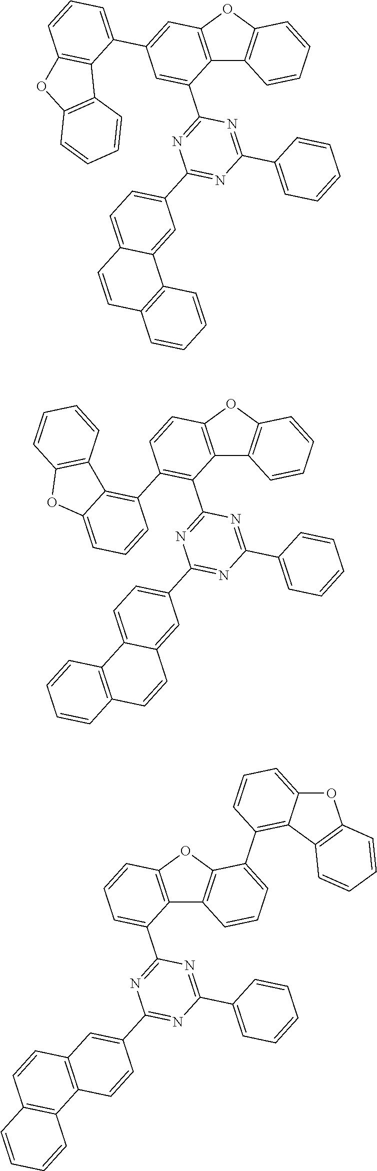 [SMILES: c1ccc(-c2nc(-c3ccc4ccc5ccccc5c4c3)nc(-c3cc(-c4cccc5oc6ccccc6c45)cc4oc5ccccc5c34)n2)cc1 ; c1ccc(-c2nc(-c3ccc4c(ccc5ccccc54)c3)nc(-c3c(-c4cccc5oc6ccccc6c45)ccc4oc5ccccc5c34)n2)cc1 ; c1ccc(-c2nc(-c3ccc4c(ccc5ccccc54)c3)nc(-c3cccc4oc5c(-c6cccc7oc8ccccc8c67)cccc5c34)n2)cc1]

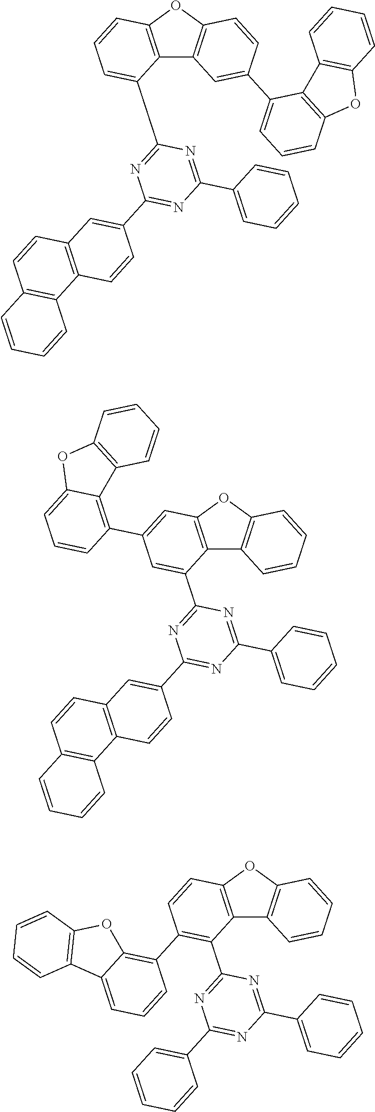 [SMILES: c1ccc(-c2nc(-c3ccc4c(ccc5ccccc54)c3)nc(-c3cccc4oc5ccc(-c6cccc7oc8ccccc8c67)cc5c34)n2)cc1 ; c1ccc(-c2nc(-c3ccc4c(ccc5ccccc54)c3)nc(-c3cc(-c4cccc5oc6ccccc6c45)cc4oc5ccccc5c34)n2)cc1 ; c1ccc(-c2nc(-c3ccccc3)nc(-c3c(-c4cccc5c4oc4ccccc45)ccc4oc5ccccc5c34)n2)cc1]

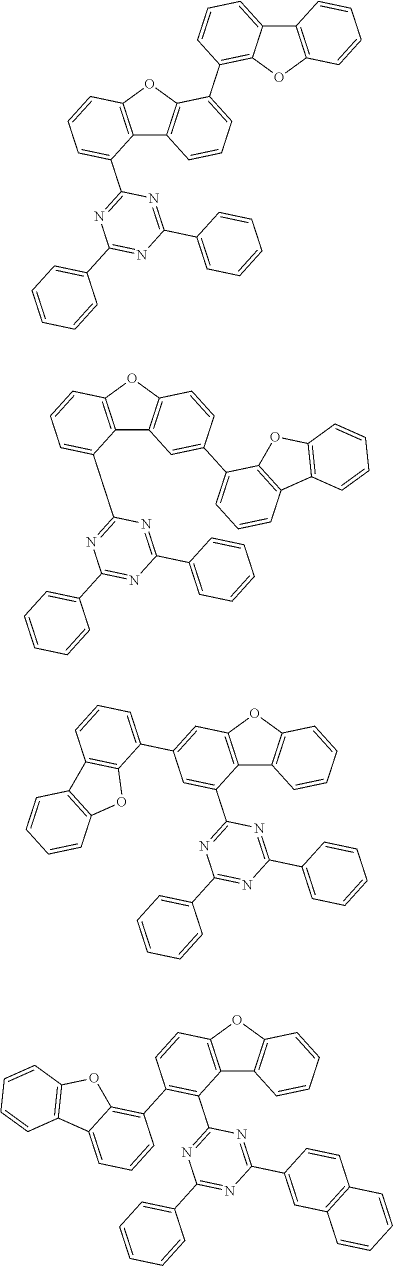 [SMILES: c1ccc(-c2nc(-c3ccccc3)nc(-c3cccc4oc5c(-c6cccc7c6oc6ccccc67)cccc5c34)n2)cc1 ; c1ccc(-c2nc(-c3ccccc3)nc(-c3cccc4oc5ccc(-c6cccc7c6oc6ccccc67)cc5c34)n2)cc1 ; c1ccc(-c2nc(-c3ccccc3)nc(-c3cc(-c4cccc5c4oc4ccccc45)cc4oc5ccccc5c34)n2)cc1 ; c1ccc(-c2nc(-c3ccc4ccccc4c3)nc(-c3c(-c4cccc5c4oc4ccccc45)ccc4oc5ccccc5c34)n2)cc1]

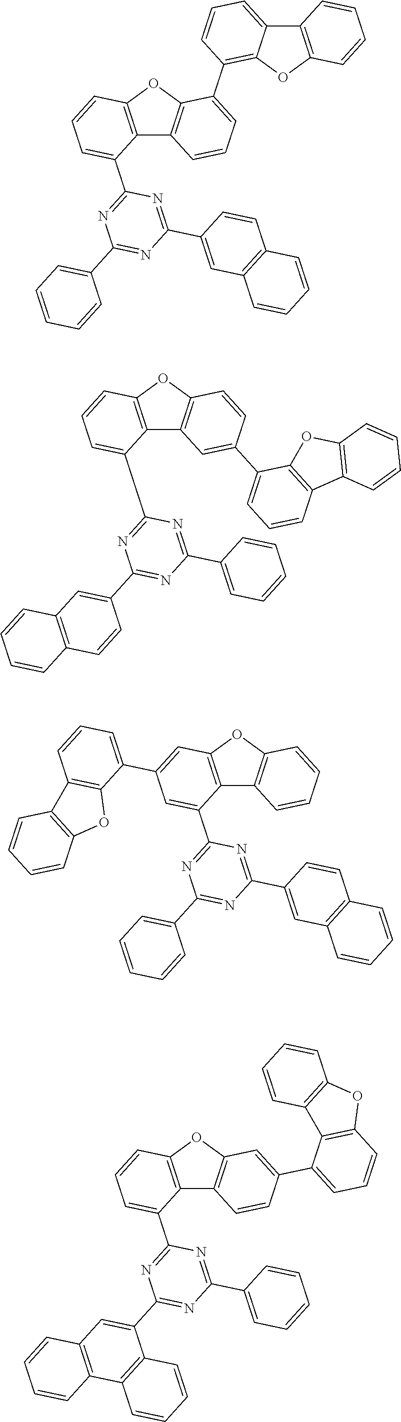 [SMILES: c1ccc(-c2nc(-c3ccc4ccccc4c3)nc(-c3cccc4oc5c(-c6cccc7c6oc6ccccc67)cccc5c34)n2)cc1 ; c1ccc(-c2nc(-c3ccc4ccccc4c3)nc(-c3cccc4oc5ccc(-c6cccc7c6oc6ccccc67)cc5c34)n2)cc1 ; c1ccc(-c2nc(-c3ccc4ccccc4c3)nc(-c3cc(-c4cccc5c4oc4ccccc45)cc4oc5ccccc5c34)n2)cc1 ; c1ccc(-c2nc(-c3cc4ccccc4c4ccccc34)nc(-c3cccc4oc5cc(-c6cccc7oc8ccccc8c67)ccc5c34)n2)cc1]

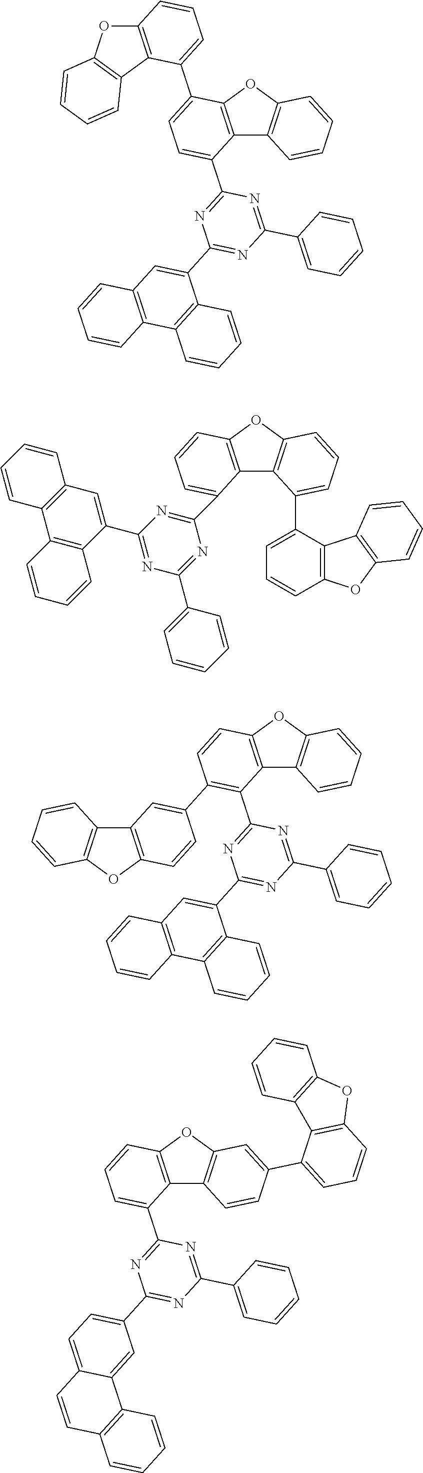 [SMILES: c1ccc(-c2nc(-c3cc4ccccc4c4ccccc34)nc(-c3ccc(-c4cccc5oc6ccccc6c45)c4oc5ccccc5c34)n2)cc1 ; c1ccc(-c2nc(-c3cc4ccccc4c4ccccc34)nc(-c3cccc4oc5cccc(-c6cccc7oc8ccccc8c67)c5c34)n2)cc1 ; c1ccc(-c2nc(-c3cc4ccccc4c4ccccc34)nc(-c3c(-c4ccc5oc6ccccc6c5c4)ccc4oc5ccccc5c34)n2)cc1 ; c1ccc(-c2nc(-c3ccc4ccc5ccccc5c4c3)nc(-c3cccc4oc5cc(-c6cccc7oc8ccccc8c67)ccc5c34)n2)cc1]

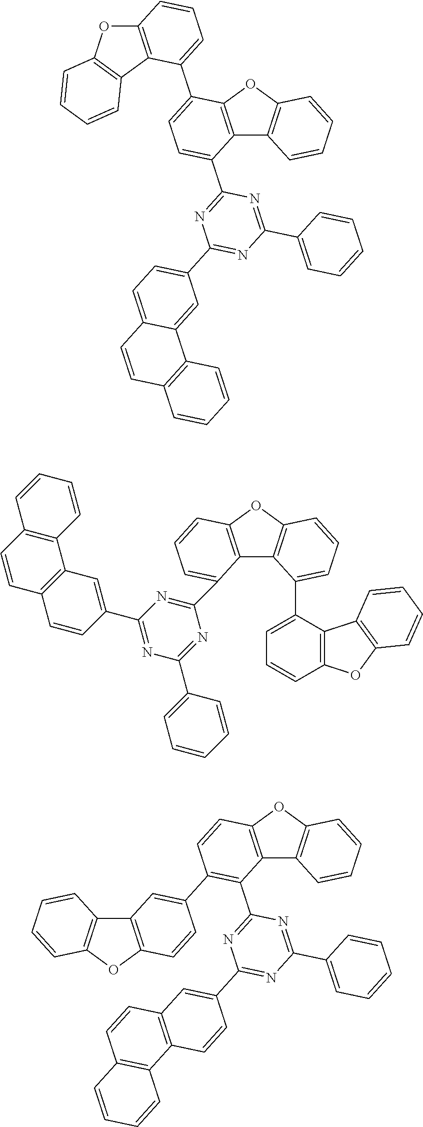 [SMILES: c1ccc(-c2nc(-c3ccc4ccc5ccccc5c4c3)nc(-c3ccc(-c4cccc5oc6ccccc6c45)c4oc5ccccc5c34)n2)cc1 ; c1ccc(-c2nc(-c3ccc4ccc5ccccc5c4c3)nc(-c3cccc4oc5cccc(-c6cccc7oc8ccccc8c67)c5c34)n2)cc1 ; c1ccc(-c2nc(-c3ccc4c(ccc5ccccc54)c3)nc(-c3c(-c4ccc5oc6ccccc6c5c4)ccc4oc5ccccc5c34)n2)cc1]

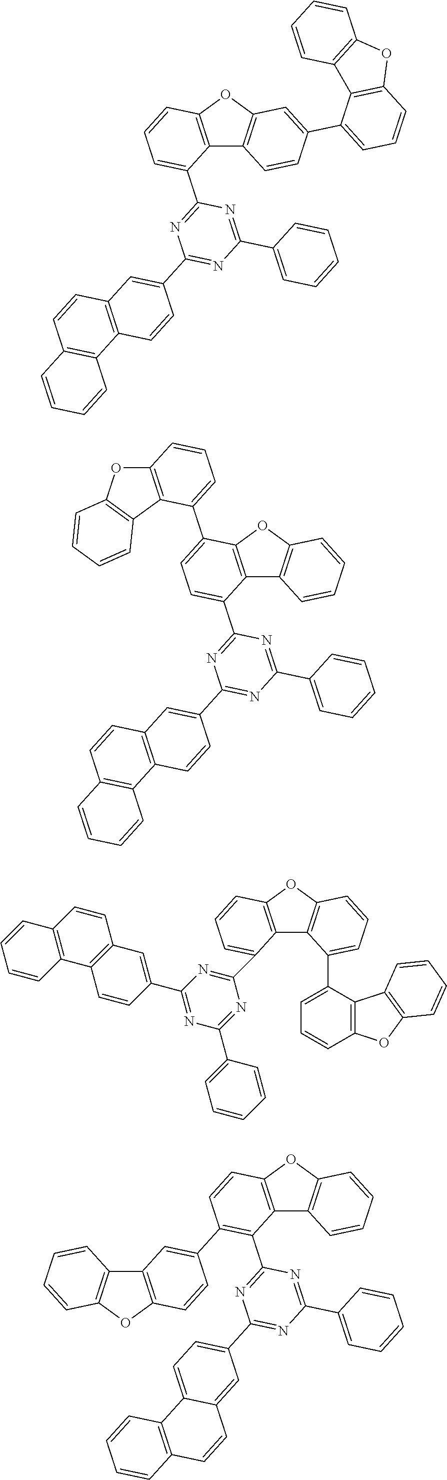 [SMILES: c1ccc(-c2nc(-c3ccc4c(ccc5ccccc54)c3)nc(-c3cccc4oc5cc(-c6cccc7oc8ccccc8c67)ccc5c34)n2)cc1 ; c1ccc(-c2nc(-c3ccc4c(ccc5ccccc54)c3)nc(-c3ccc(-c4cccc5oc6ccccc6c45)c4oc5ccccc5c34)n2)cc1 ; c1ccc(-c2nc(-c3ccc4c(ccc5ccccc54)c3)nc(-c3cccc4oc5cccc(-c6cccc7oc8ccccc8c67)c5c34)n2)cc1 ; c1ccc(-c2nc(-c3ccc4c(ccc5ccccc54)c3)nc(-c3c(-c4ccc5oc6ccccc6c5c4)ccc4oc5ccccc5c34)n2)cc1]

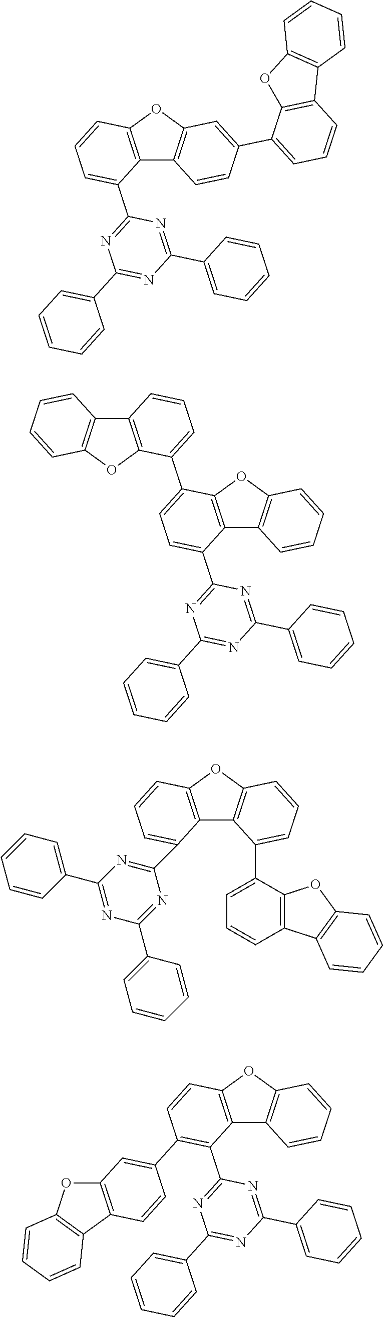

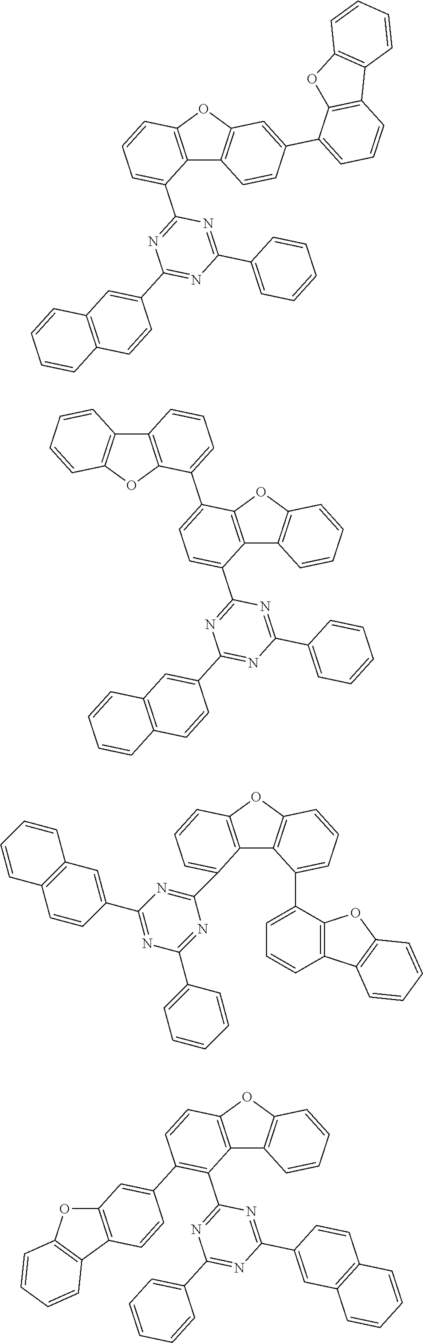

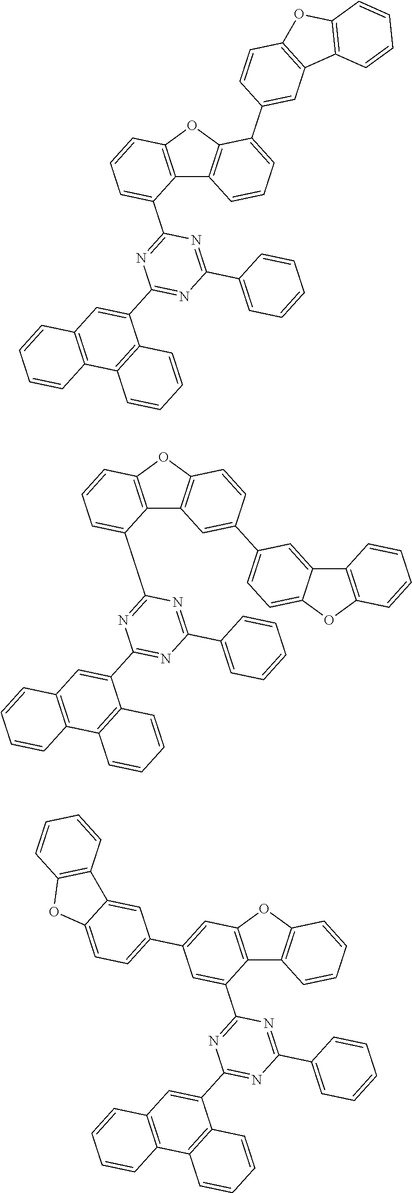 [SMILES: c1ccc(-c2nc(-c3cc4ccccc4c4ccccc34)nc(-c3cccc4oc5c(-c6ccc7oc8ccccc8c7c6)cccc5c34)n2)cc1 ; c1ccc(-c2nc(-c3cc4ccccc4c4ccccc34)nc(-c3cccc4oc5ccc(-c6ccc7oc8ccccc8c7c6)cc5c34)n2)cc1 ; c1ccc(-c2nc(-c3cc4ccccc4c4ccccc34)nc(-c3cc(-c4ccc5oc6ccccc6c5c4)cc4oc5ccccc5c34)n2)cc1]

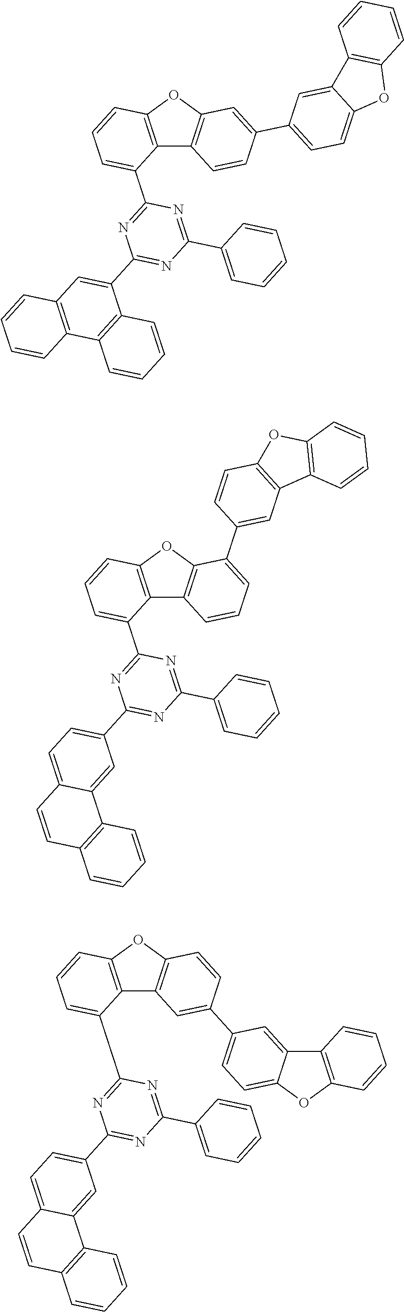 [SMILES: c1ccc(-c2nc(-c3cc4ccccc4c4ccccc34)nc(-c3cccc4oc5cc(-c6ccc7oc8ccccc8c7c6)ccc5c34)n2)cc1 ; c1ccc(-c2nc(-c3ccc4ccc5ccccc5c4c3)nc(-c3cccc4oc5c(-c6ccc7oc8ccccc8c7c6)cccc5c34)n2)cc1 ; c1ccc(-c2nc(-c3ccc4ccc5ccccc5c4c3)nc(-c3cccc4oc5ccc(-c6ccc7oc8ccccc8c7c6)cc5c34)n2)cc1]

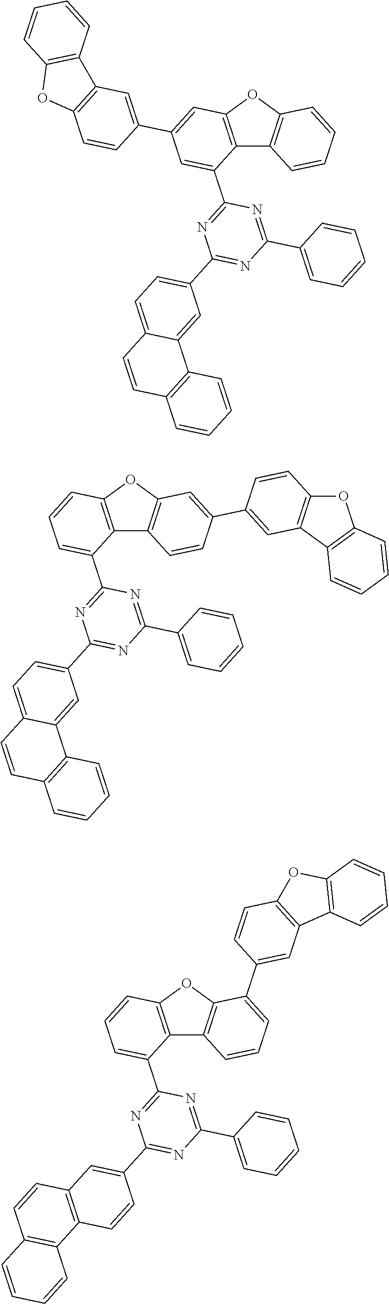 [SMILES: c1ccc(-c2nc(-c3ccc4ccc5ccccc5c4c3)nc(-c3cc(-c4ccc5oc6ccccc6c5c4)cc4oc5ccccc5c34)n2)cc1 ; c1ccc(-c2nc(-c3ccc4ccc5ccccc5c4c3)nc(-c3cccc4oc5cc(-c6ccc7oc8ccccc8c7c6)ccc5c34)n2)cc1 ; c1ccc(-c2nc(-c3ccc4c(ccc5ccccc54)c3)nc(-c3cccc4oc5c(-c6ccc7oc8ccccc8c7c6)cccc5c34)n2)cc1]

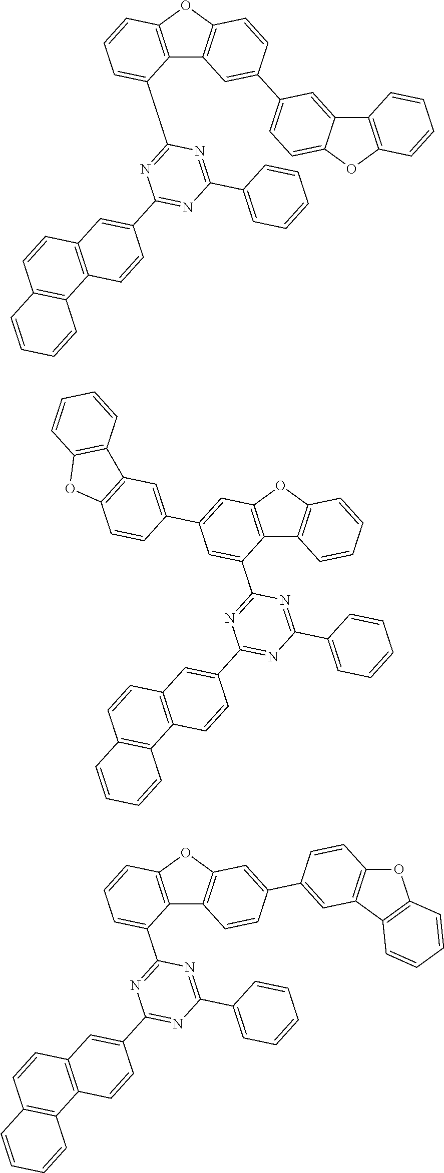

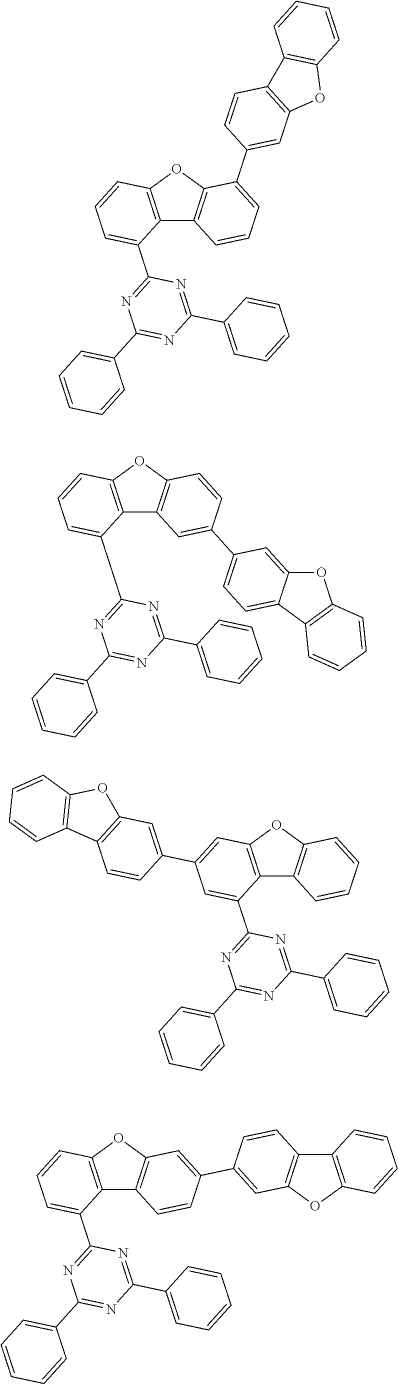

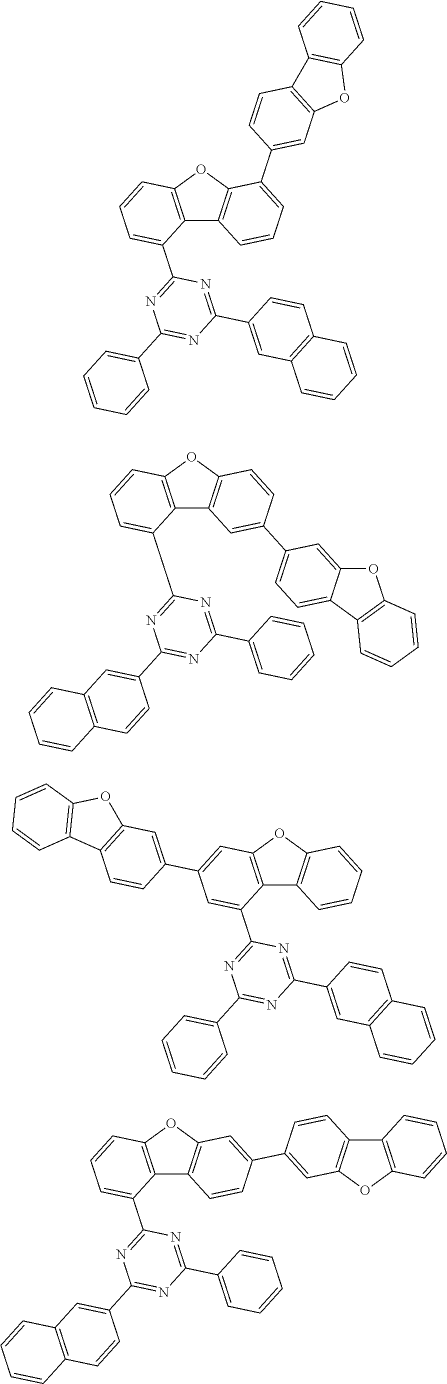

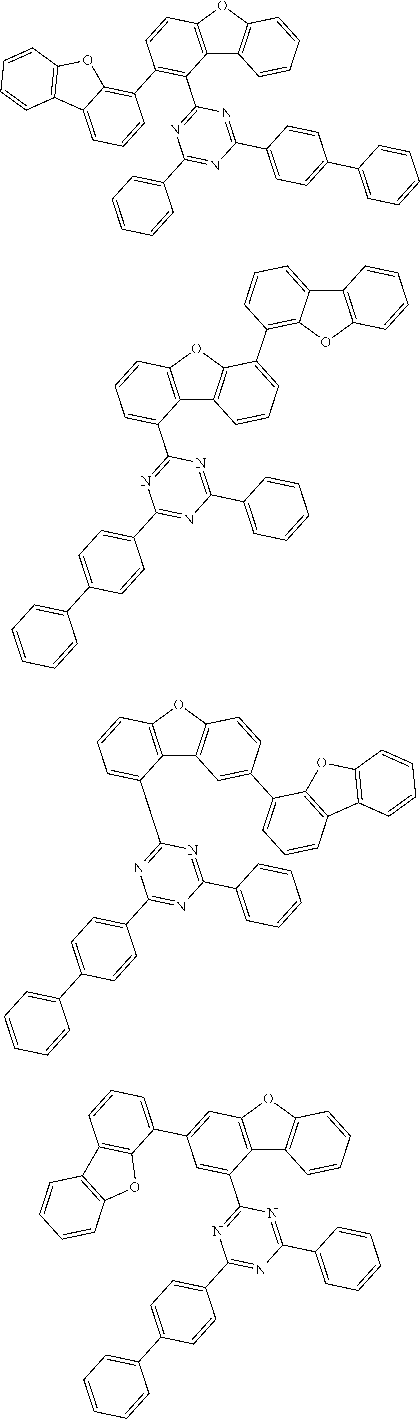 [SMILES: c1ccc(-c2ccc(-c3nc(-c4ccccc4)nc(-c4c(-c5cccc6c5oc5ccccc56)ccc5oc6ccccc6c45)n3)cc2)cc1 ; c1ccc(-c2ccc(-c3nc(-c4ccccc4)nc(-c4cccc5oc6c(-c7cccc8c7oc7ccccc78)cccc6c45)n3)cc2)cc1 ; c1ccc(-c2ccc(-c3nc(-c4ccccc4)nc(-c4cccc5oc6ccc(-c7cccc8c7oc7ccccc78)cc6c45)n3)cc2)cc1 ; c1ccc(-c2ccc(-c3nc(-c4ccccc4)nc(-c4cc(-c5cccc6c5oc5ccccc56)cc5oc6ccccc6c45)n3)cc2)cc1]

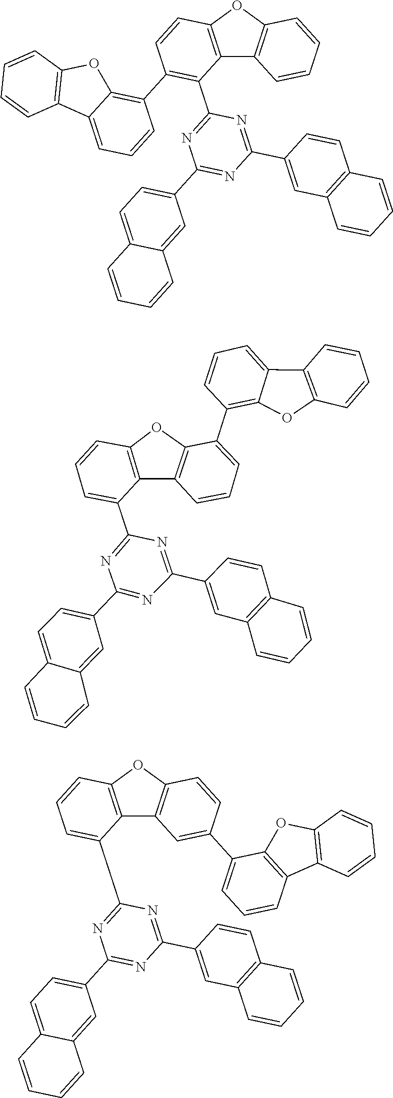 [SMILES: c1ccc2cc(-c3nc(-c4ccc5ccccc5c4)nc(-c4c(-c5cccc6c5oc5ccccc56)ccc5oc6ccccc6c45)n3)ccc2c1 ; c1ccc2cc(-c3nc(-c4ccc5ccccc5c4)nc(-c4cccc5oc6c(-c7cccc8c7oc7ccccc78)cccc6c45)n3)ccc2c1 ; c1ccc2cc(-c3nc(-c4ccc5ccccc5c4)nc(-c4cccc5oc6ccc(-c7cccc8c7oc7ccccc78)cc6c45)n3)ccc2c1]

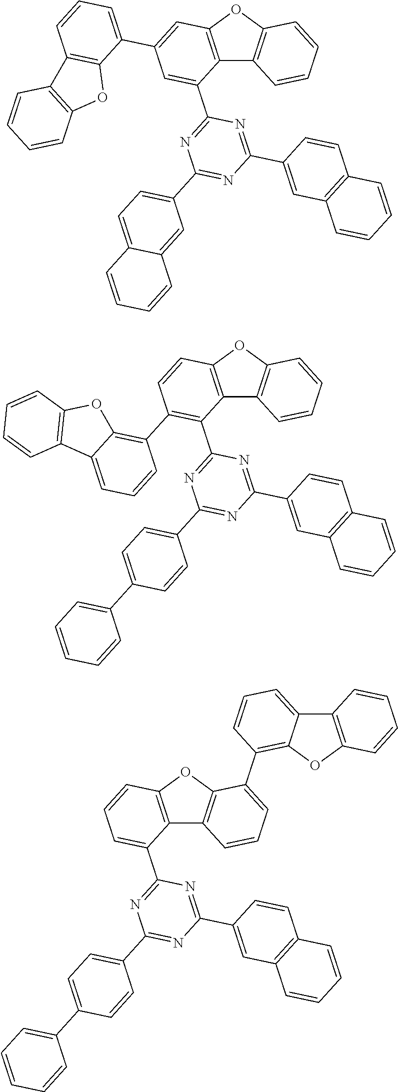 [SMILES: c1ccc2cc(-c3nc(-c4ccc5ccccc5c4)nc(-c4cc(-c5cccc6c5oc5ccccc56)cc5oc6ccccc6c45)n3)ccc2c1 ; c1ccc(-c2ccc(-c3nc(-c4ccc5ccccc5c4)nc(-c4c(-c5cccc6c5oc5ccccc56)ccc5oc6ccccc6c45)n3)cc2)cc1 ; c1ccc(-c2ccc(-c3nc(-c4ccc5ccccc5c4)nc(-c4cccc5oc6c(-c7cccc8c7oc7ccccc78)cccc6c45)n3)cc2)cc1]

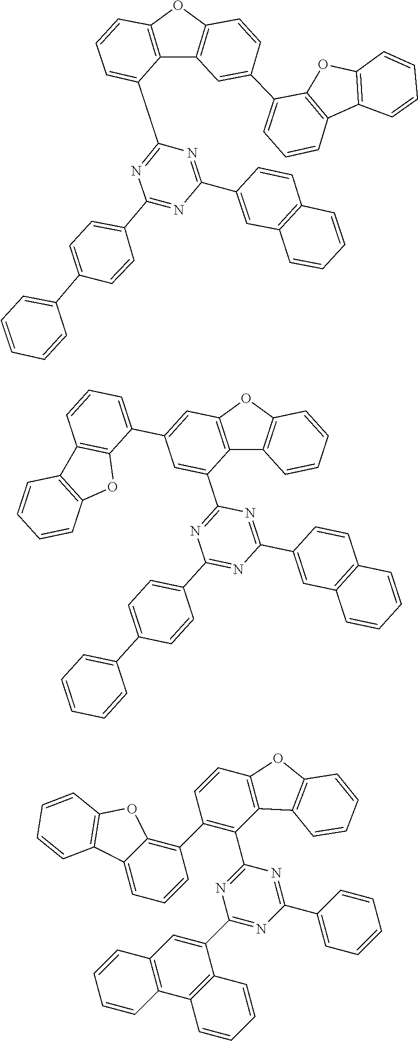 [SMILES: c1ccc(-c2ccc(-c3nc(-c4ccc5ccccc5c4)nc(-c4cccc5oc6ccc(-c7cccc8c7oc7ccccc78)cc6c45)n3)cc2)cc1 ; c1ccc(-c2ccc(-c3nc(-c4ccc5ccccc5c4)nc(-c4cc(-c5cccc6c5oc5ccccc56)cc5oc6ccccc6c45)n3)cc2)cc1 ; c1ccc(-c2nc(-c3cc4ccccc4c4ccccc34)nc(-c3c(-c4cccc5c4oc4ccccc45)ccc4oc5ccccc5c34)n2)cc1]

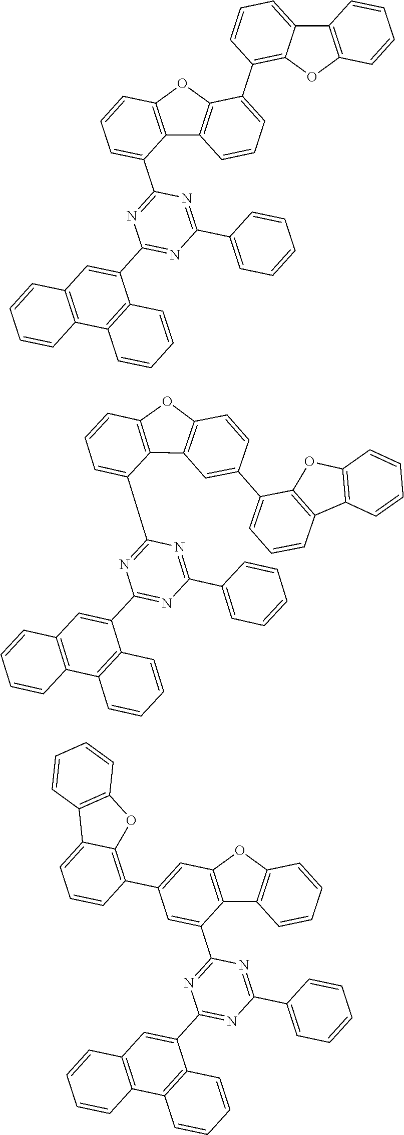

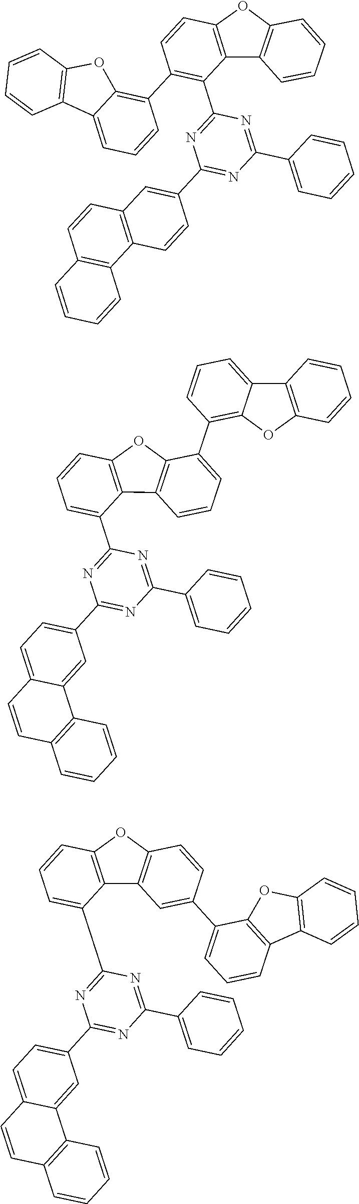 [SMILES: c1ccc(-c2nc(-c3ccc4c(ccc5ccccc54)c3)nc(-c3c(-c4cccc5c4oc4ccccc45)ccc4oc5ccccc5c34)n2)cc1 ; c1ccc(-c2nc(-c3ccc4ccc5ccccc5c4c3)nc(-c3cccc4oc5c(-c6cccc7c6oc6ccccc67)cccc5c34)n2)cc1 ; c1ccc(-c2nc(-c3ccc4ccc5ccccc5c4c3)nc(-c3cccc4oc5ccc(-c6cccc7c6oc6ccccc67)cc5c34)n2)cc1]

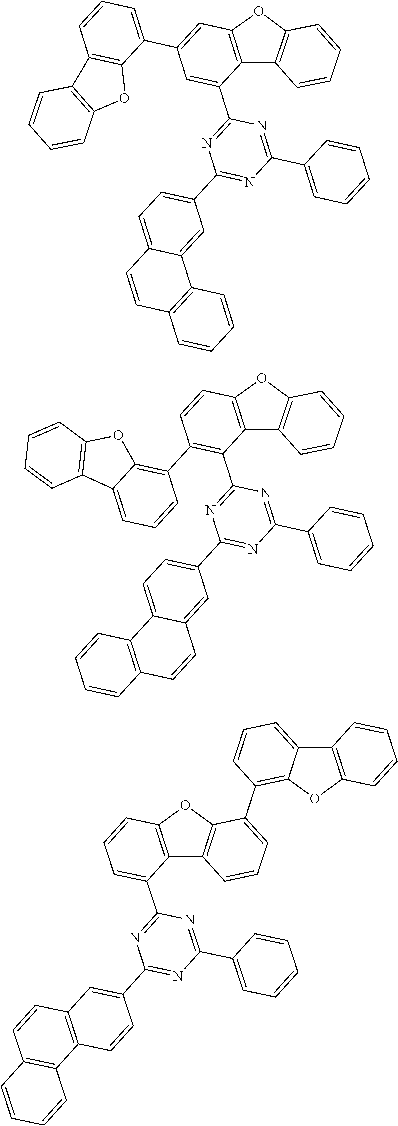

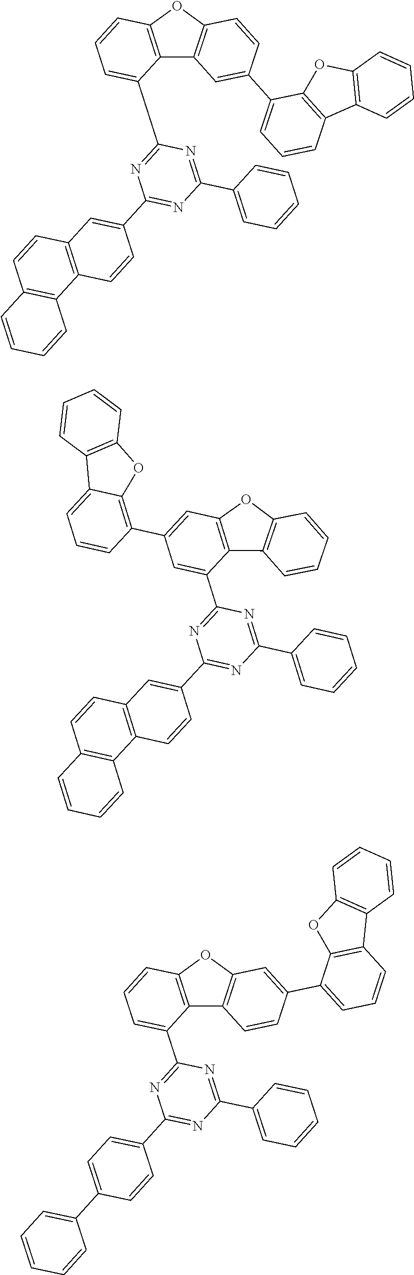 [SMILES: c1ccc(-c2nc(-c3ccc4c(ccc5ccccc54)c3)nc(-c3cccc4oc5ccc(-c6cccc7c6oc6ccccc67)cc5c34)n2)cc1 ; c1ccc(-c2nc(-c3ccc4c(ccc5ccccc54)c3)nc(-c3cc(-c4cccc5c4oc4ccccc45)cc4oc5ccccc5c34)n2)cc1 ; c1ccc(-c2ccc(-c3nc(-c4ccccc4)nc(-c4cccc5oc6cc(-c7cccc8c7oc7ccccc78)ccc6c45)n3)cc2)cc1]

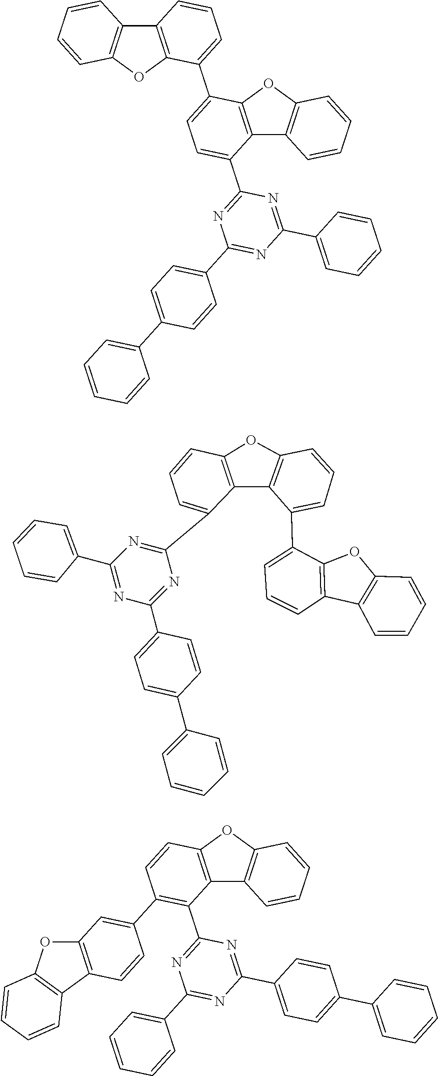

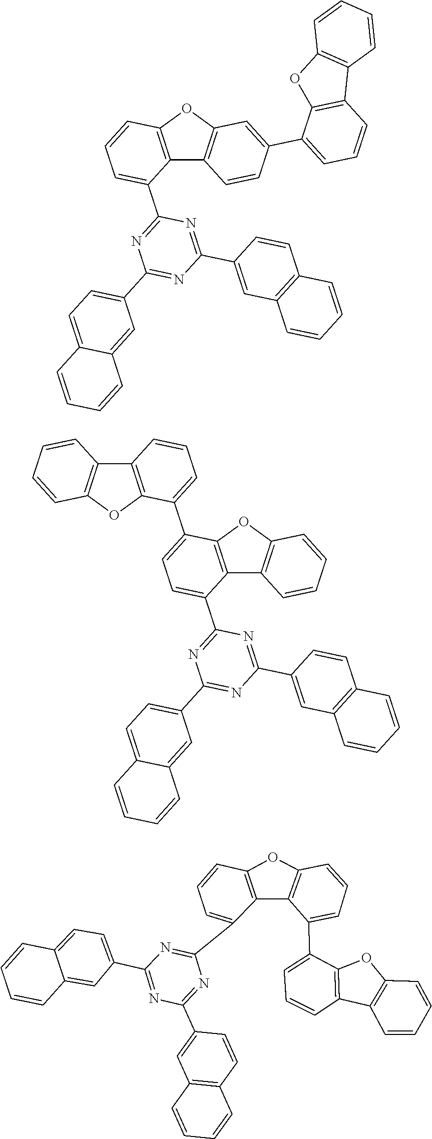

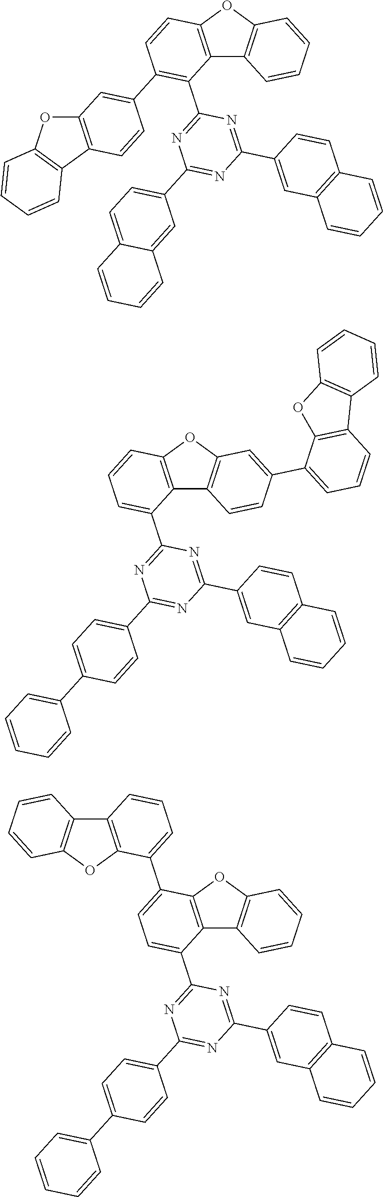

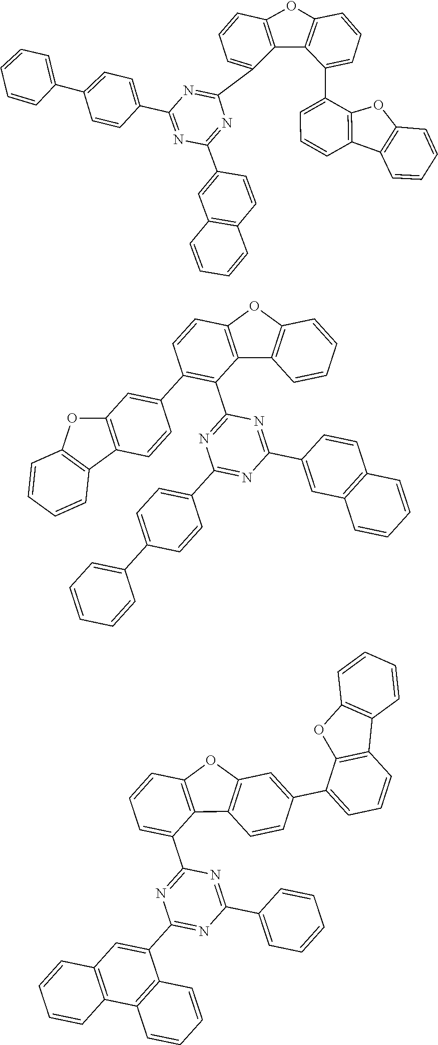

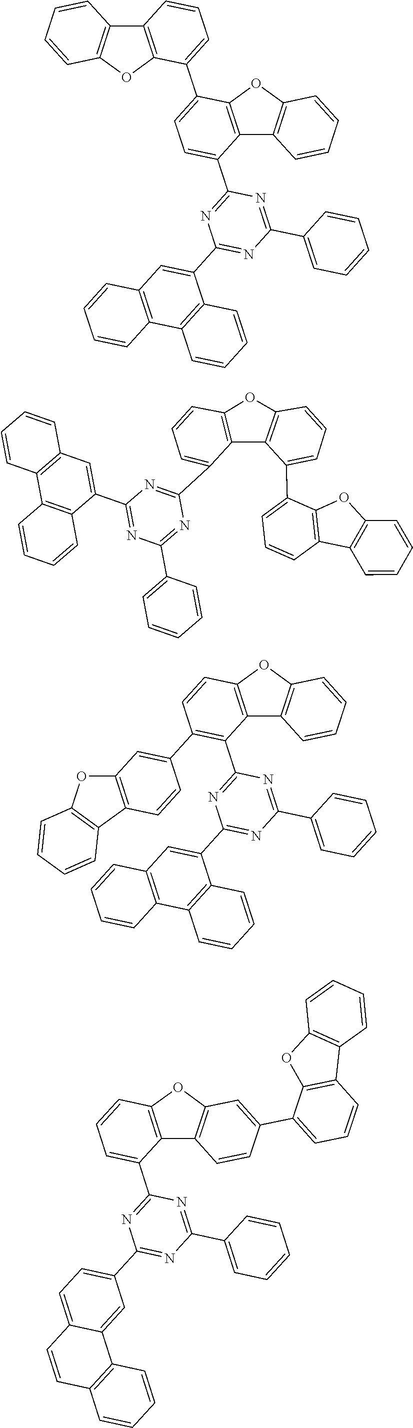

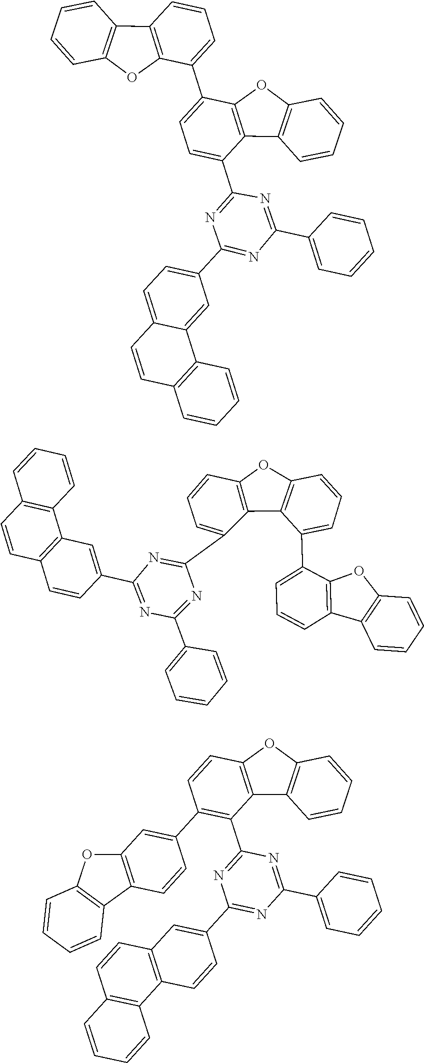 [SMILES: c1ccc(-c2nc(-c3ccc4ccc5ccccc5c4c3)nc(-c3ccc(-c4cccc5c4oc4ccccc45)c4oc5ccccc5c34)n2)cc1 ; c1ccc(-c2nc(-c3ccc4ccc5ccccc5c4c3)nc(-c3cccc4oc5cccc(-c6cccc7c6oc6ccccc67)c5c34)n2)cc1 ; c1ccc(-c2nc(-c3ccc4c(ccc5ccccc54)c3)nc(-c3c(-c4ccc5c(c4)oc4ccccc45)ccc4oc5ccccc5c34)n2)cc1]

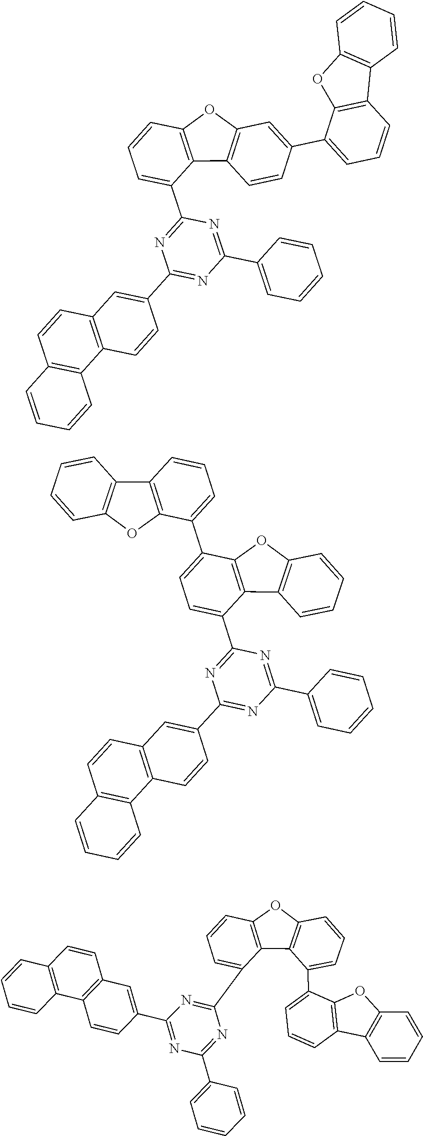

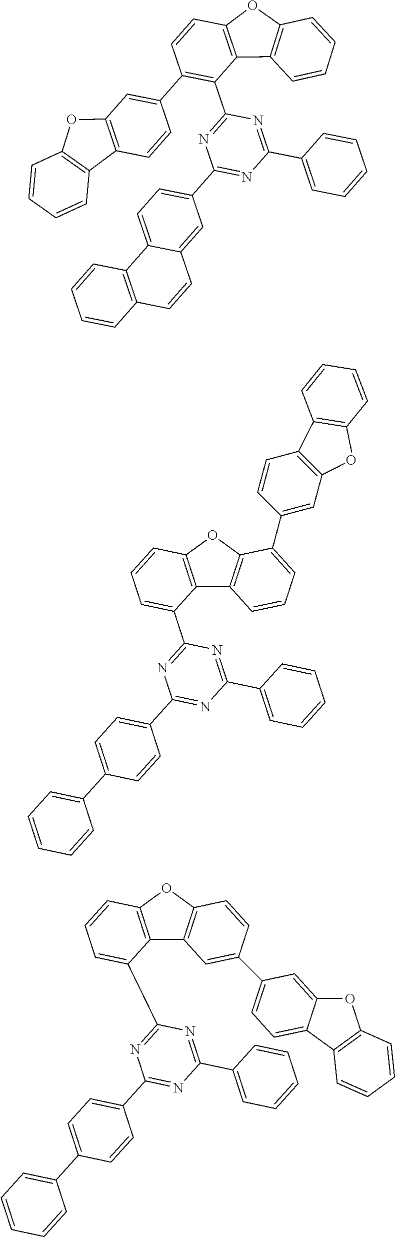 [SMILES: c1ccc(-c2nc(-c3ccc4c(ccc5ccccc54)c3)nc(-c3c(-c4ccc5c(c4)oc4ccccc45)ccc4oc5ccccc5c34)n2)cc1 ; c1ccc(-c2ccc(-c3nc(-c4ccccc4)nc(-c4cccc5oc6c(-c7ccc8c(c7)oc7ccccc78)cccc6c45)n3)cc2)cc1 ; c1ccc(-c2ccc(-c3nc(-c4ccccc4)nc(-c4cccc5oc6ccc(-c7ccc8c(c7)oc7ccccc78)cc6c45)n3)cc2)cc1]

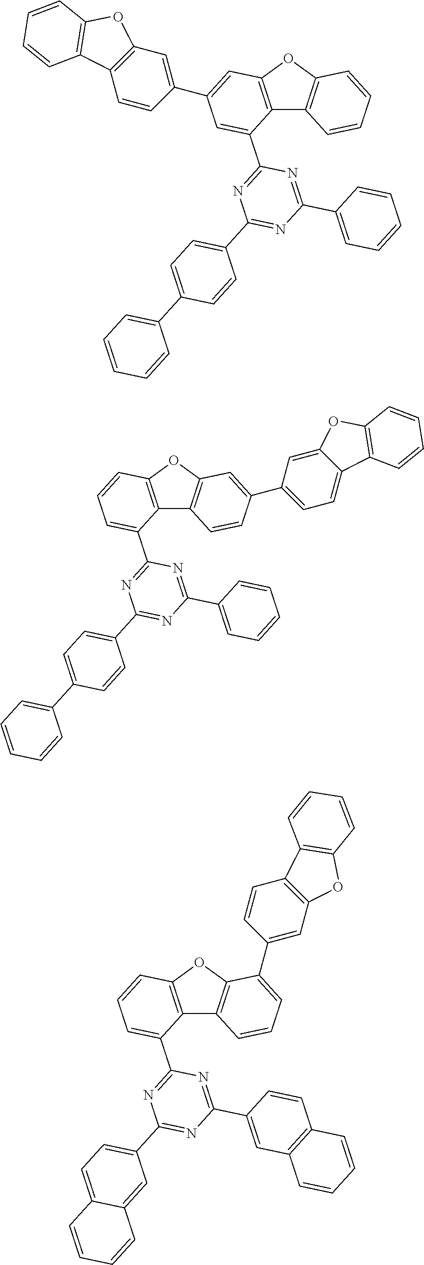 [SMILES: c1ccc(-c2ccc(-c3nc(-c4ccccc4)nc(-c4cc(-c5ccc6c(c5)oc5ccccc56)cc5oc6ccccc6c45)n3)cc2)cc1 ; c1ccc(-c2ccc(-c3nc(-c4ccccc4)nc(-c4cccc5oc6cc(-c7ccc8c(c7)oc7ccccc78)ccc6c45)n3)cc2)cc1 ; c1ccc2cc(-c3nc(-c4ccc5ccccc5c4)nc(-c4cccc5oc6c(-c7ccc8c(c7)oc7ccccc78)cccc6c45)n3)ccc2c1]

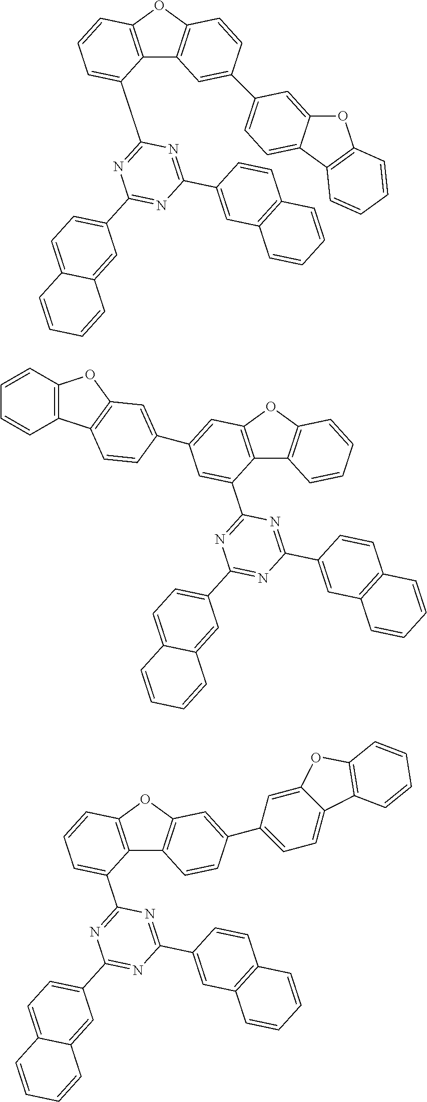

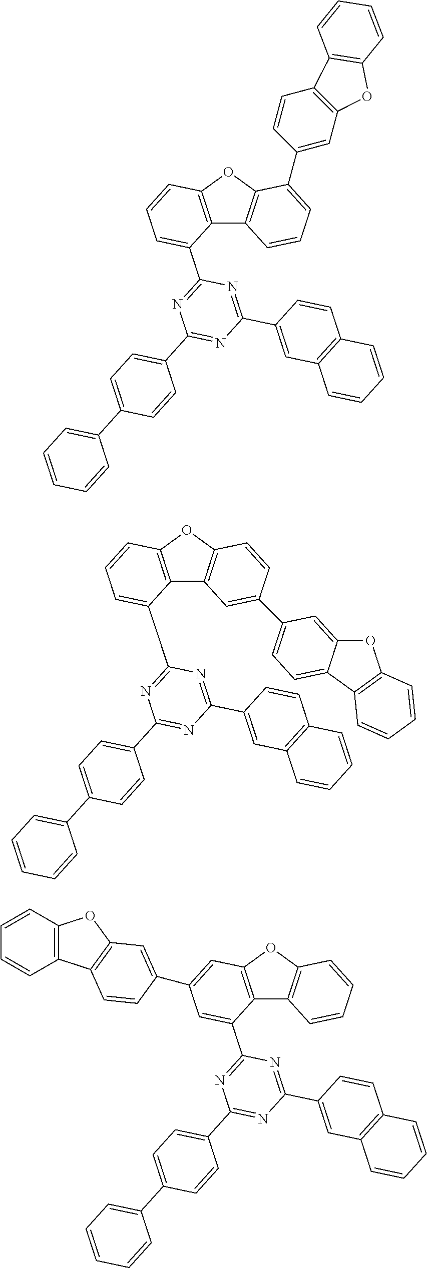

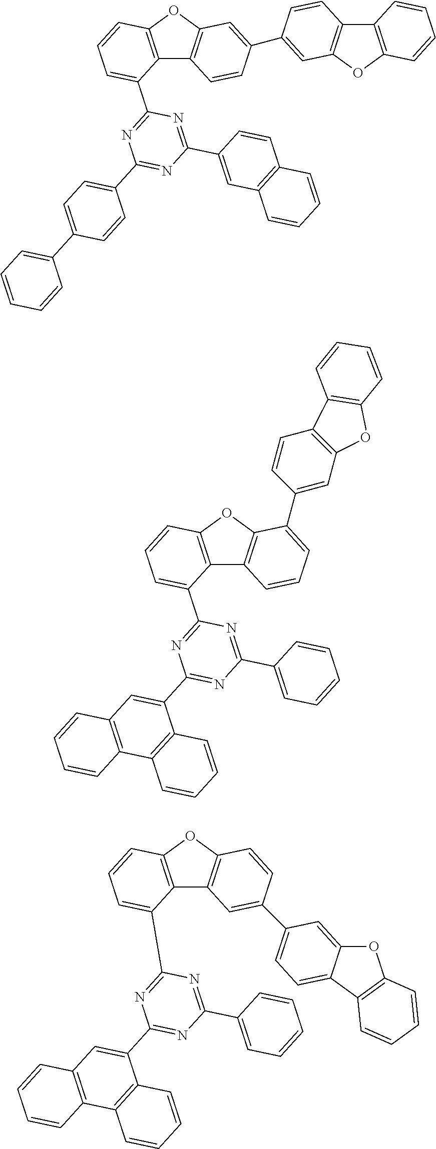

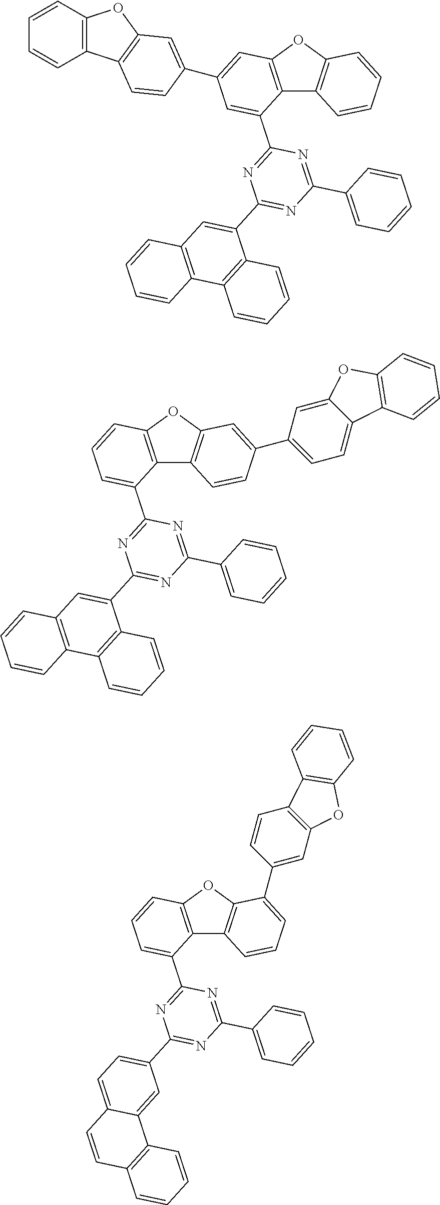 [SMILES: c1ccc(-c2nc(-c3cc4ccccc4c4ccccc34)nc(-c3cc(-c4ccc5c(c4)oc4ccccc45)cc4oc5ccccc5c34)n2)cc1 ; c1ccc(-c2nc(-c3cc4ccccc4c4ccccc34)nc(-c3cccc4oc5cc(-c6ccc7c(c6)oc6ccccc67)ccc5c34)n2)cc1 ; c1ccc(-c2nc(-c3ccc4ccc5ccccc5c4c3)nc(-c3cccc4oc5c(-c6ccc7c(c6)oc6ccccc67)cccc5c34)n2)cc1]

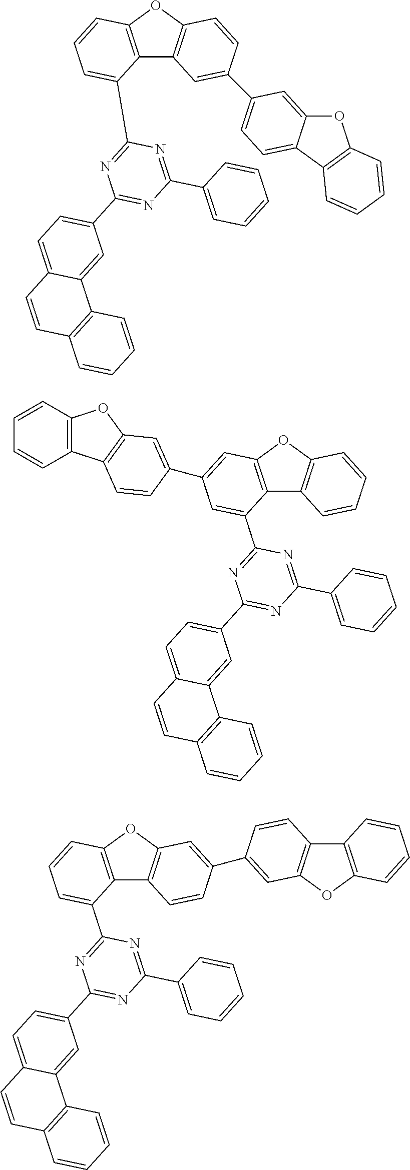

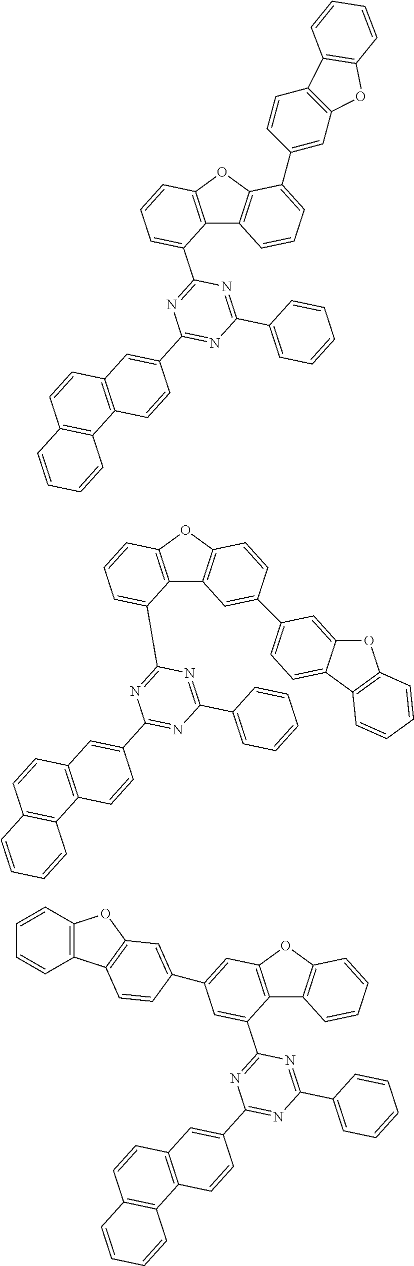

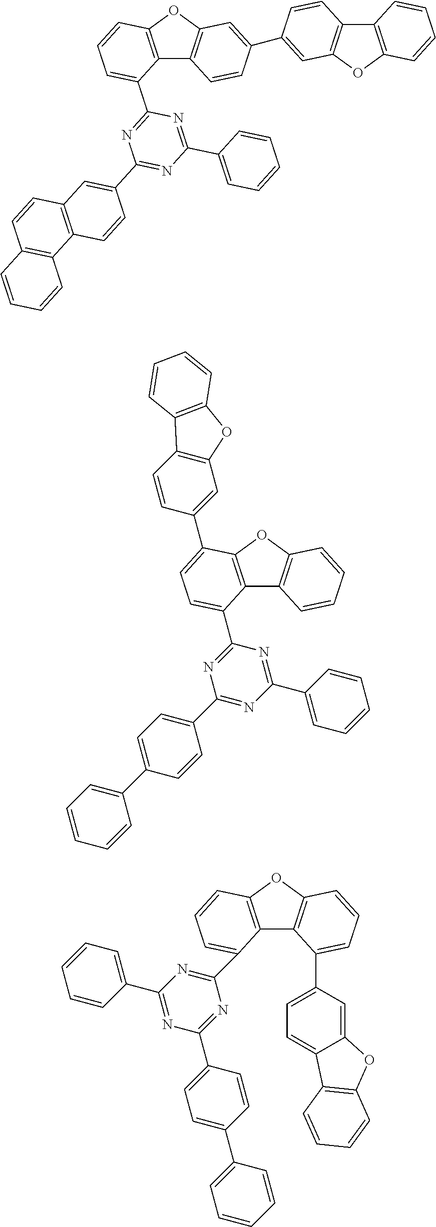 [SMILES: c1ccc(-c2nc(-c3ccc4c(ccc5ccccc54)c3)nc(-c3cccc4oc5cc(-c6ccc7c(c6)oc6ccccc67)ccc5c34)n2)cc1 ; c1ccc(-c2ccc(-c3nc(-c4ccccc4)nc(-c4ccc(-c5ccc6c(c5)oc5ccccc56)c5oc6ccccc6c45)n3)cc2)cc1 ; c1ccc(-c2ccc(-c3nc(-c4ccccc4)nc(-c4cccc5oc6cccc(-c7ccc8c(c7)oc7ccccc78)c6c45)n3)cc2)cc1]

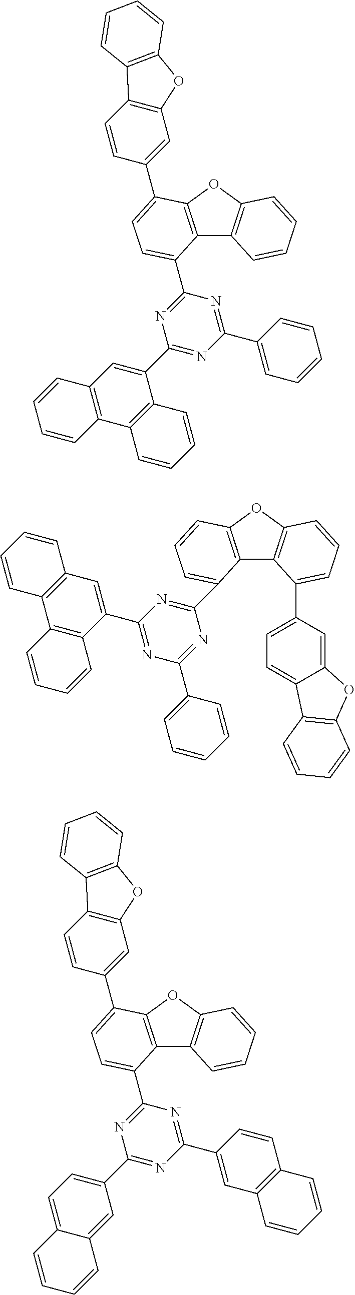 [SMILES: c1ccc(-c2nc(-c3cc4ccccc4c4ccccc34)nc(-c3ccc(-c4ccc5c(c4)oc4ccccc45)c4oc5ccccc5c34)n2)cc1 ; c1ccc(-c2nc(-c3cc4ccccc4c4ccccc34)nc(-c3cccc4oc5cccc(-c6ccc7c(c6)oc6ccccc67)c5c34)n2)cc1 ; c1ccc2cc(-c3nc(-c4ccc5ccccc5c4)nc(-c4ccc(-c5ccc6c(c5)oc5ccccc56)c5oc6ccccc6c45)n3)ccc2c1]

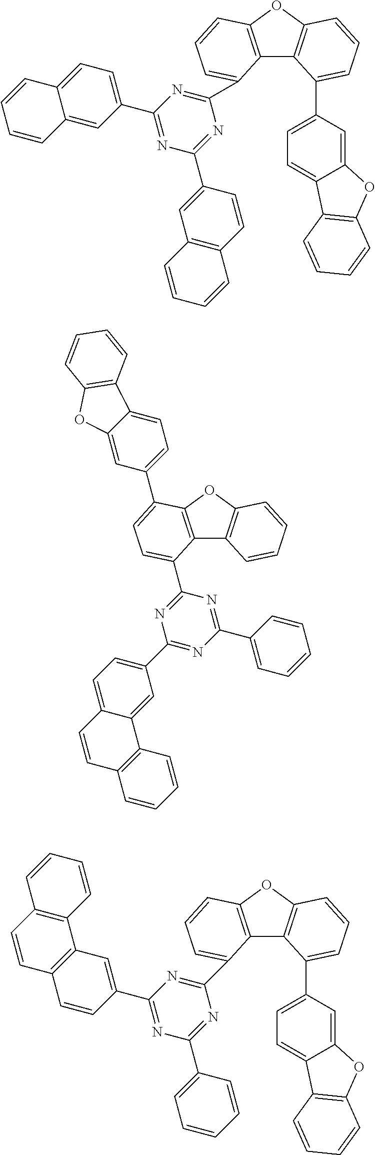 [SMILES: c1ccc2cc(-c3nc(-c4ccc5ccccc5c4)nc(-c4cccc5oc6cccc(-c7ccc8c(c7)oc7ccccc78)c6c45)n3)ccc2c1 ; c1ccc(-c2nc(-c3ccc4ccc5ccccc5c4c3)nc(-c3ccc(-c4ccc5c(c4)oc4ccccc45)c4oc5ccccc5c34)n2)cc1 ; c1ccc(-c2nc(-c3ccc4ccc5ccccc5c4c3)nc(-c3cccc4oc5cccc(-c6ccc7c(c6)oc6ccccc67)c5c34)n2)cc1]

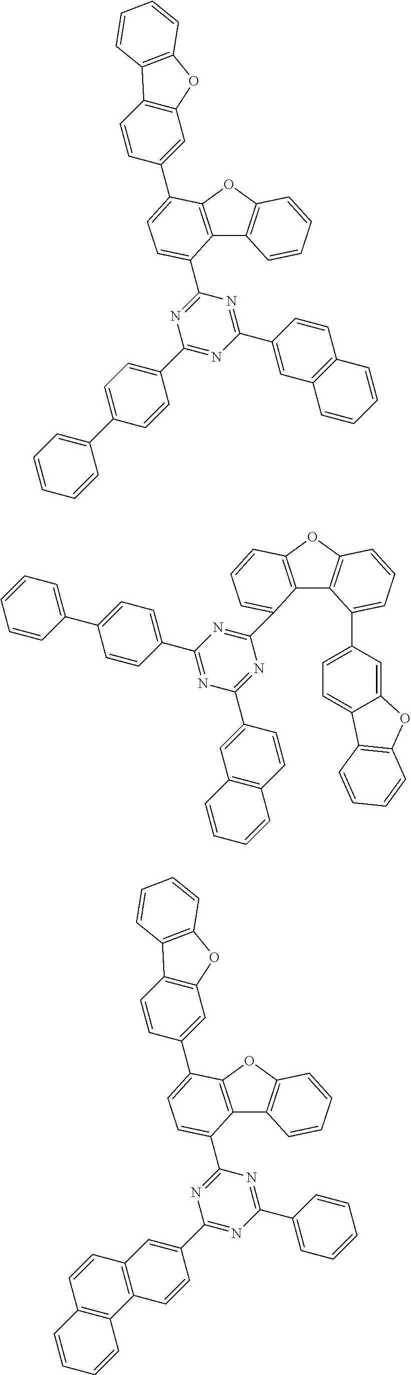 [SMILES: c1ccc(-c2ccc(-c3nc(-c4ccc5ccccc5c4)nc(-c4ccc(-c5ccc6c(c5)oc5ccccc56)c5oc6ccccc6c45)n3)cc2)cc1 ; c1ccc(-c2ccc(-c3nc(-c4ccc5ccccc5c4)nc(-c4cccc5oc6cccc(-c7ccc8c(c7)oc7ccccc78)c6c45)n3)cc2)cc1 ; c1ccc(-c2nc(-c3ccc4c(ccc5ccccc54)c3)nc(-c3ccc(-c4ccc5c(c4)oc4ccccc45)c4oc5ccccc5c34)n2)cc1]

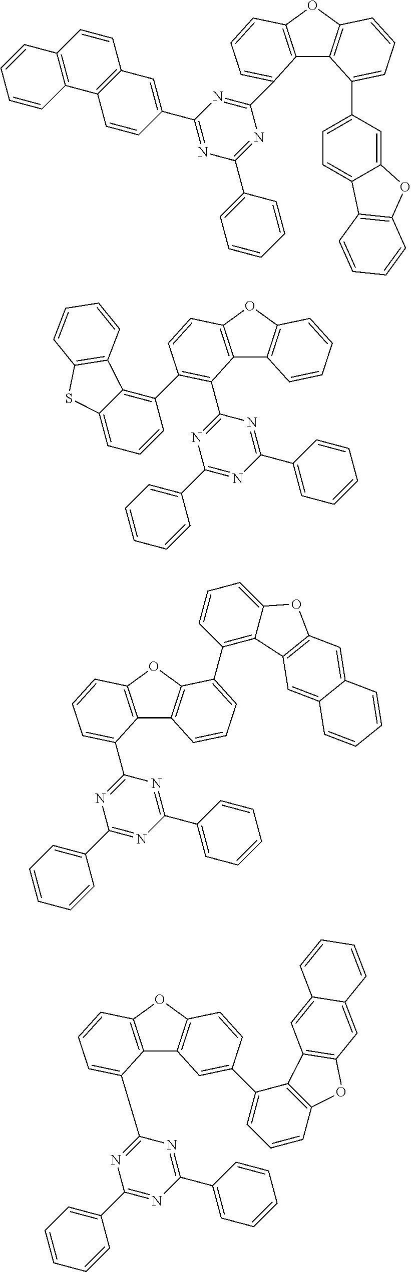 [SMILES: c1ccc(-c2nc(-c3ccc4c(ccc5ccccc54)c3)nc(-c3cccc4oc5cccc(-c6ccc7c(c6)oc6ccccc67)c5c34)n2)cc1 ; c1ccc(-c2nc(-c3ccccc3)nc(-c3c(-c4cccc5sc6ccccc6c45)ccc4oc5ccccc5c34)n2)cc1 ; c1ccc(-c2nc(-c3ccccc3)nc(-c3cccc4oc5c(-c6cccc7oc8cc9ccccc9cc8c67)cccc5c34)n2)cc1 ; c1ccc(-c2nc(-c3ccccc3)nc(-c3cccc4oc5ccc(-c6cccc7oc8cc9ccccc9cc8c67)cc5c34)n2)cc1]

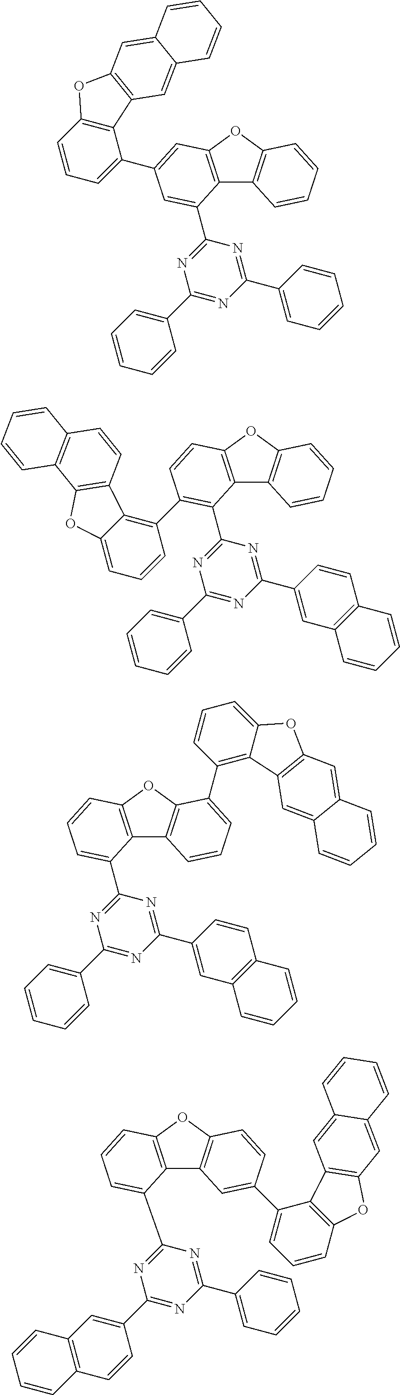 [SMILES: c1ccc(-c2nc(-c3ccccc3)nc(-c3cc(-c4cccc5oc6cc7ccccc7cc6c45)cc4oc5ccccc5c34)n2)cc1 ; c1ccc(-c2nc(-c3ccc4ccccc4c3)nc(-c3c(-c4cccc5oc6c7ccccc7ccc6c45)ccc4oc5ccccc5c34)n2)cc1 ; c1ccc(-c2nc(-c3ccc4ccccc4c3)nc(-c3cccc4oc5c(-c6cccc7oc8cc9ccccc9cc8c67)cccc5c34)n2)cc1 ; c1ccc(-c2nc(-c3ccc4ccccc4c3)nc(-c3cccc4oc5ccc(-c6cccc7oc8cc9ccccc9cc8c67)cc5c34)n2)cc1]

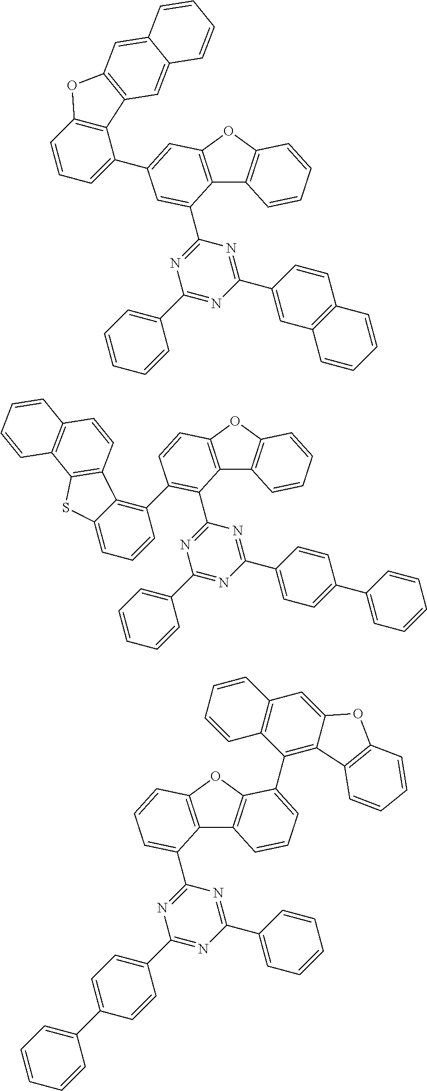 [SMILES: c1ccc(-c2nc(-c3ccc4ccccc4c3)nc(-c3cc(-c4cccc5oc6cc7ccccc7cc6c45)cc4oc5ccccc5c34)n2)cc1 ; c1ccc(-c2ccc(-c3nc(-c4ccccc4)nc(-c4c(-c5cccc6sc7c8ccccc8ccc7c56)ccc5oc6ccccc6c45)n3)cc2)cc1 ; c1ccc(-c2ccc(-c3nc(-c4ccccc4)nc(-c4cccc5oc6c(-c7c8ccccc8cc8oc9ccccc9c78)cccc6c45)n3)cc2)cc1]

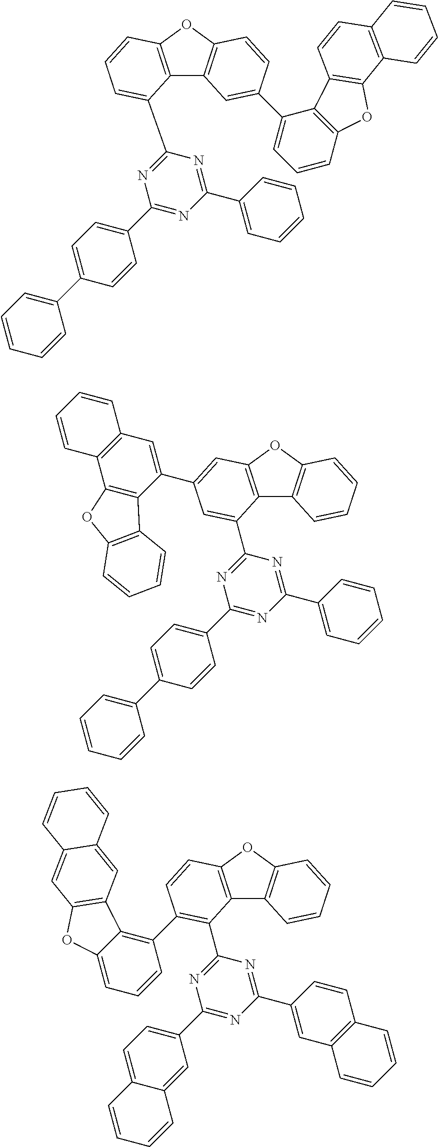

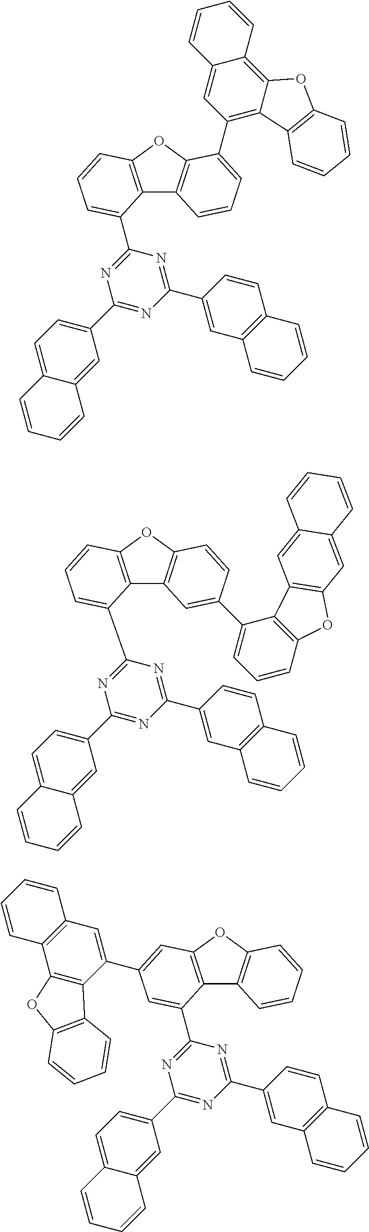

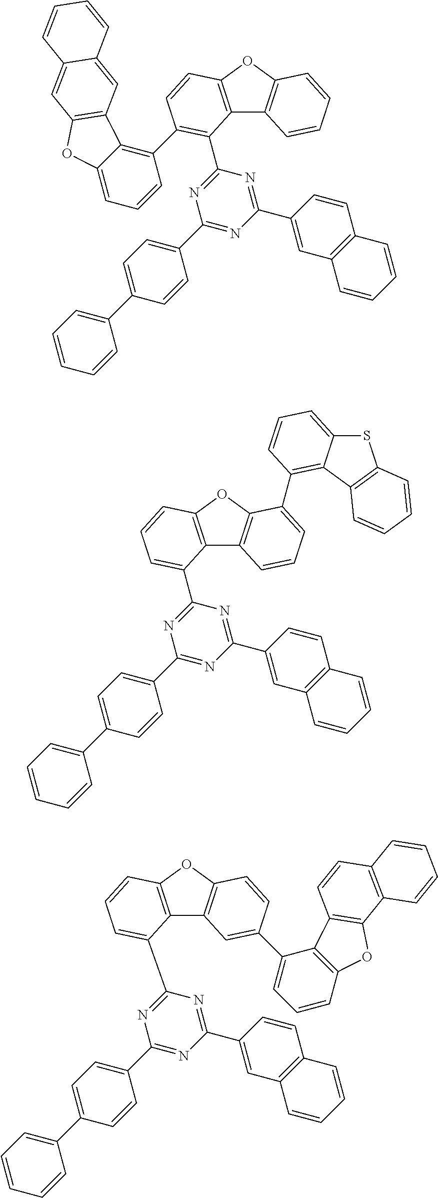 [SMILES: c1ccc(-c2ccc(-c3nc(-c4ccc5ccccc5c4)nc(-c4c(-c5cccc6oc7cc8ccccc8cc7c56)ccc5oc6ccccc6c45)n3)cc2)cc1 ; c1ccc(-c2ccc(-c3nc(-c4ccc5ccccc5c4)nc(-c4cccc5oc6c(-c7cccc8sc9ccccc9c78)cccc6c45)n3)cc2)cc1 ; c1ccc(-c2ccc(-c3nc(-c4ccc5ccccc5c4)nc(-c4cccc5oc6ccc(-c7cccc8oc9c%10ccccc%10ccc9c78)cc6c45)n3)cc2)cc1]

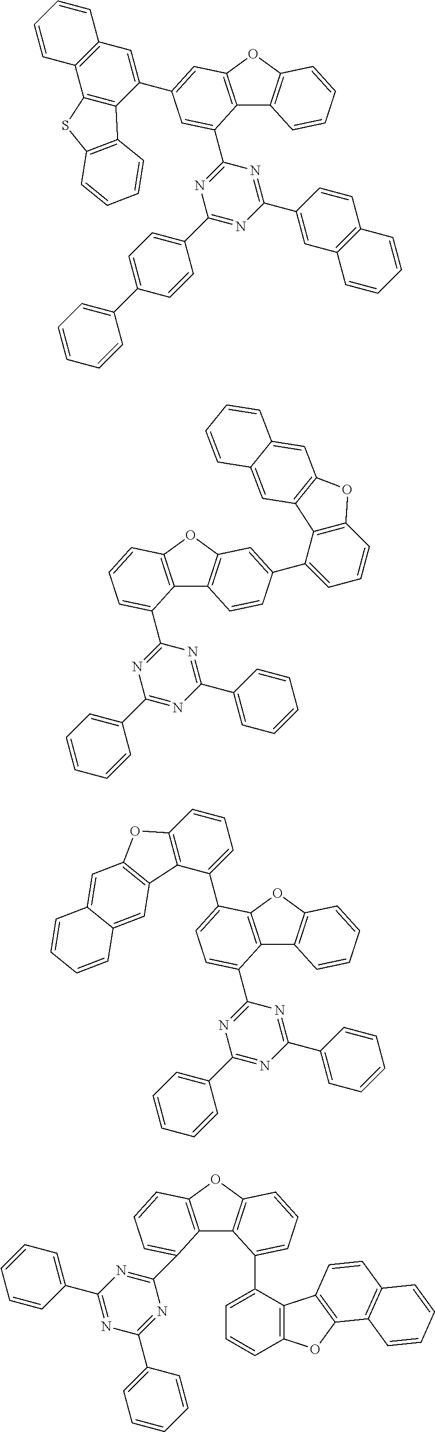 [SMILES: c1ccc(-c2ccc(-c3nc(-c4ccc5ccccc5c4)nc(-c4cc(-c5cc6ccccc6c6sc7ccccc7c56)cc5oc6ccccc6c45)n3)cc2)cc1 ; c1ccc(-c2nc(-c3ccccc3)nc(-c3cccc4oc5cc(-c6cccc7oc8cc9ccccc9cc8c67)ccc5c34)n2)cc1 ; c1ccc(-c2nc(-c3ccccc3)nc(-c3ccc(-c4cccc5oc6cc7ccccc7cc6c45)c4oc5ccccc5c34)n2)cc1 ; c1ccc(-c2nc(-c3ccccc3)nc(-c3cccc4oc5cccc(-c6cccc7oc8c9ccccc9ccc8c67)c5c34)n2)cc1]

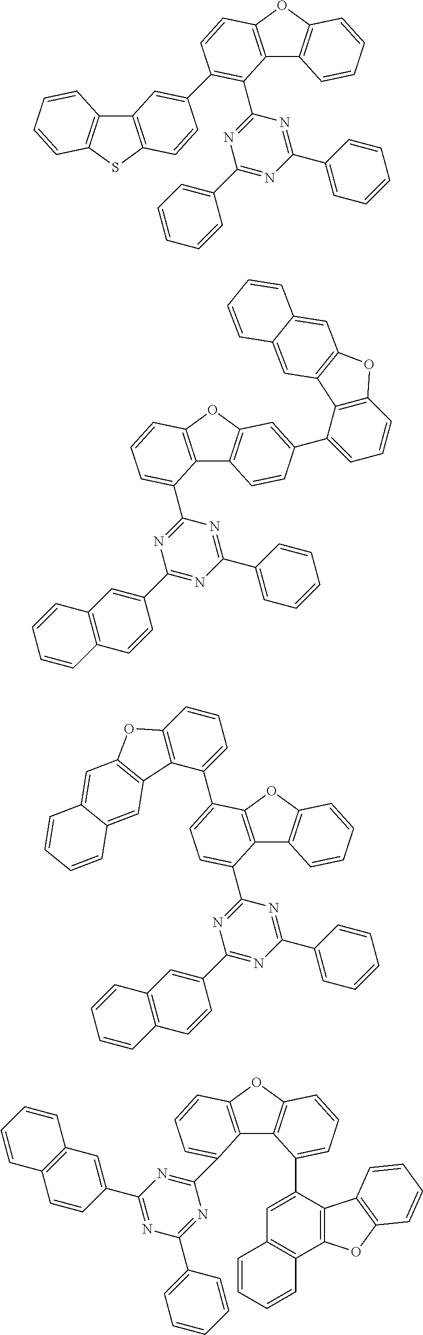 [SMILES: c1ccc(-c2nc(-c3ccccc3)nc(-c3c(-c4ccc5sc6ccccc6c5c4)ccc4oc5ccccc5c34)n2)cc1 ; c1ccc(-c2nc(-c3ccc4ccccc4c3)nc(-c3cccc4oc5cc(-c6cccc7oc8cc9ccccc9cc8c67)ccc5c34)n2)cc1 ; c1ccc(-c2nc(-c3ccc4ccccc4c3)nc(-c3ccc(-c4cccc5oc6cc7ccccc7cc6c45)c4oc5ccccc5c34)n2)cc1 ; c1ccc(-c2nc(-c3ccc4ccccc4c3)nc(-c3cccc4oc5cccc(-c6cc7ccccc7c7oc8ccccc8c67)c5c34)n2)cc1]

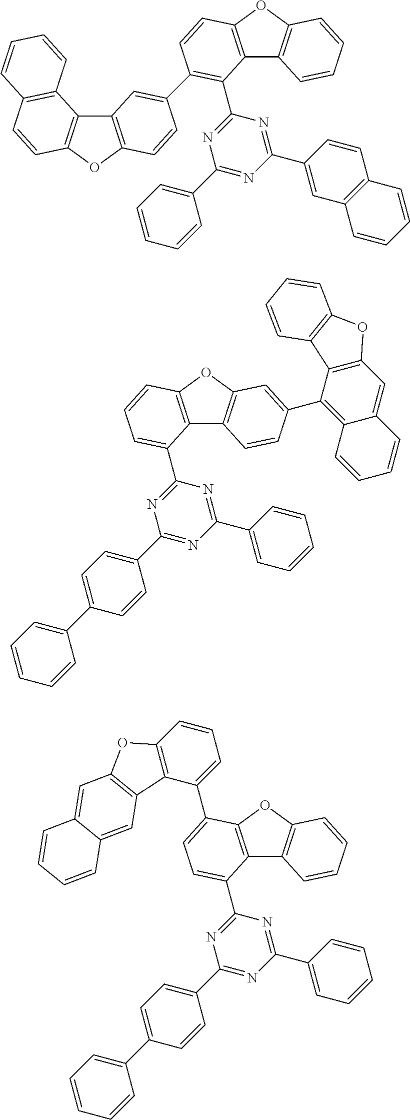 [SMILES: c1ccc(-c2nc(-c3ccc4ccccc4c3)nc(-c3c(-c4ccc5oc6ccc7ccccc7c6c5c4)ccc4oc5ccccc5c34)n2)cc1 ; c1ccc(-c2ccc(-c3nc(-c4ccccc4)nc(-c4cccc5oc6cc(-c7c8ccccc8cc8oc9ccccc9c78)ccc6c45)n3)cc2)cc1 ; c1ccc(-c2ccc(-c3nc(-c4ccccc4)nc(-c4ccc(-c5cccc6oc7cc8ccccc8cc7c56)c5oc6ccccc6c45)n3)cc2)cc1]

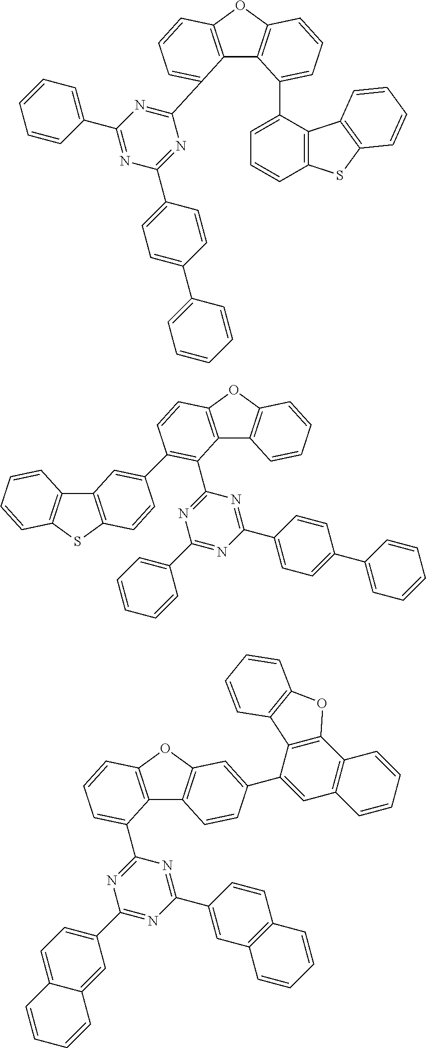

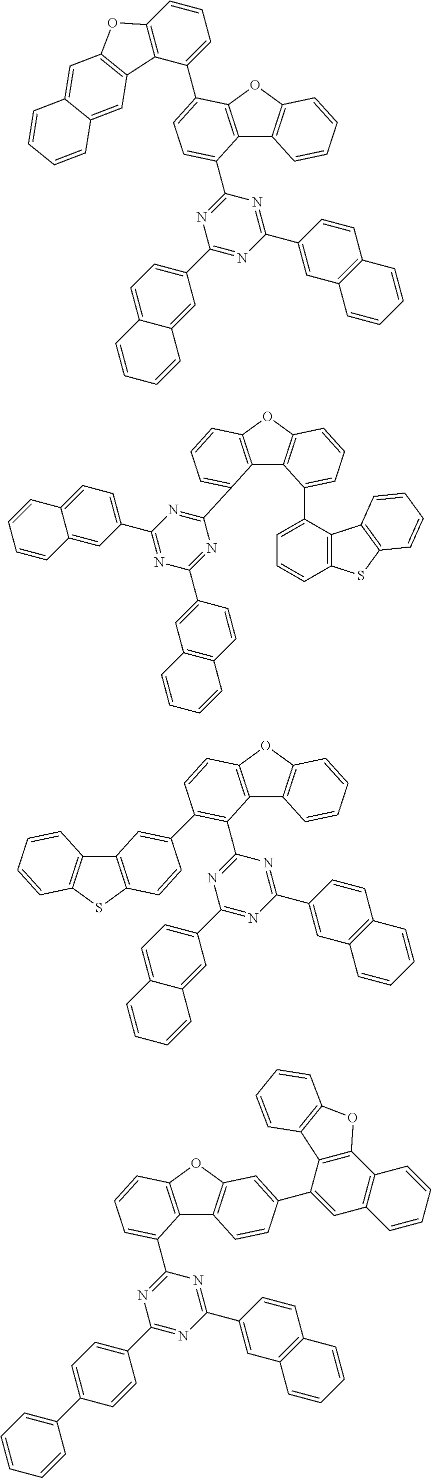

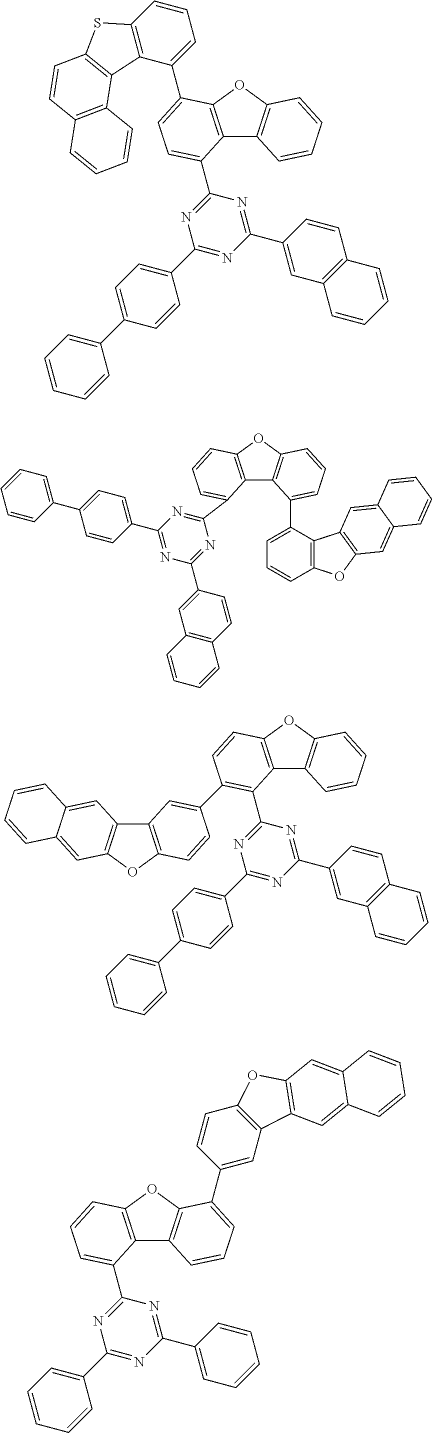 [SMILES: c1ccc(-c2ccc(-c3nc(-c4ccc5ccccc5c4)nc(-c4ccc(-c5cccc6sc7ccc8ccccc8c7c56)c5oc6ccccc6c45)n3)cc2)cc1 ; c1ccc(-c2ccc(-c3nc(-c4ccc5ccccc5c4)nc(-c4cccc5oc6cccc(-c7cccc8oc9cc%10ccccc%10cc9c78)c6c45)n3)cc2)cc1 ; c1ccc(-c2ccc(-c3nc(-c4ccc5ccccc5c4)nc(-c4c(-c5ccc6oc7cc8ccccc8cc7c6c5)ccc5oc6ccccc6c45)n3)cc2)cc1 ; c1ccc(-c2nc(-c3ccccc3)nc(-c3cccc4oc5c(-c6ccc7oc8cc9ccccc9cc8c7c6)cccc5c34)n2)cc1]

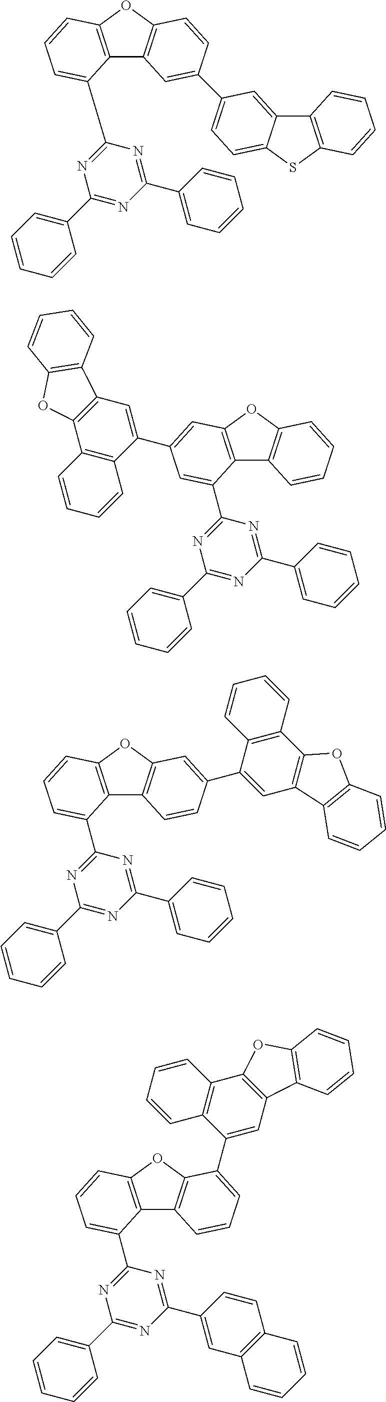

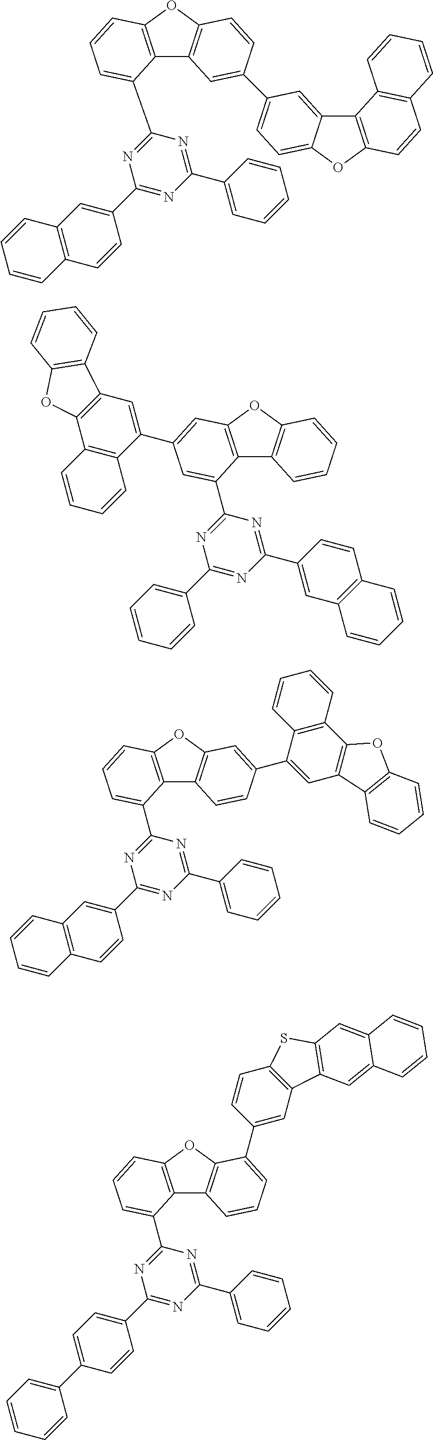

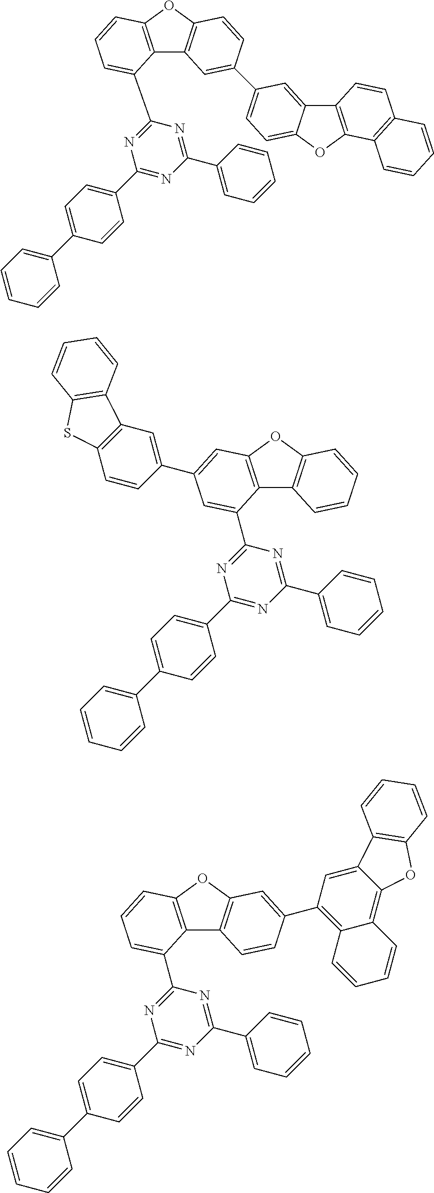

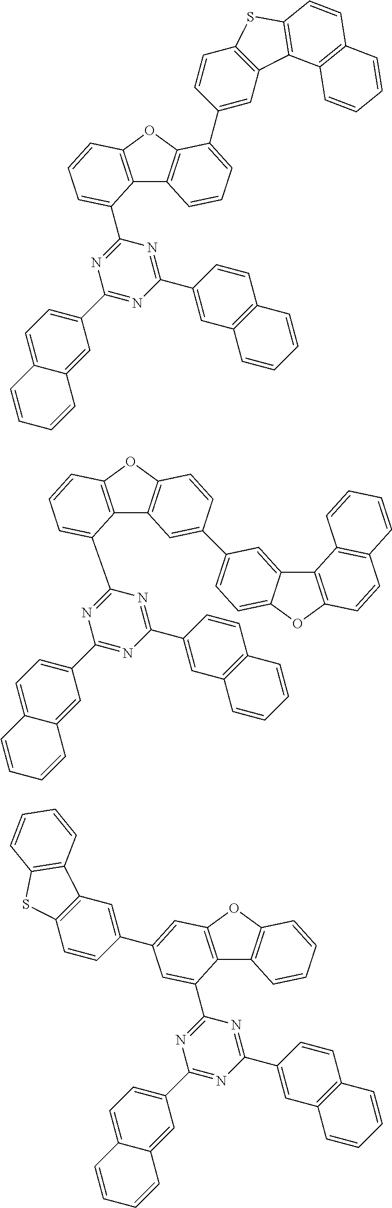

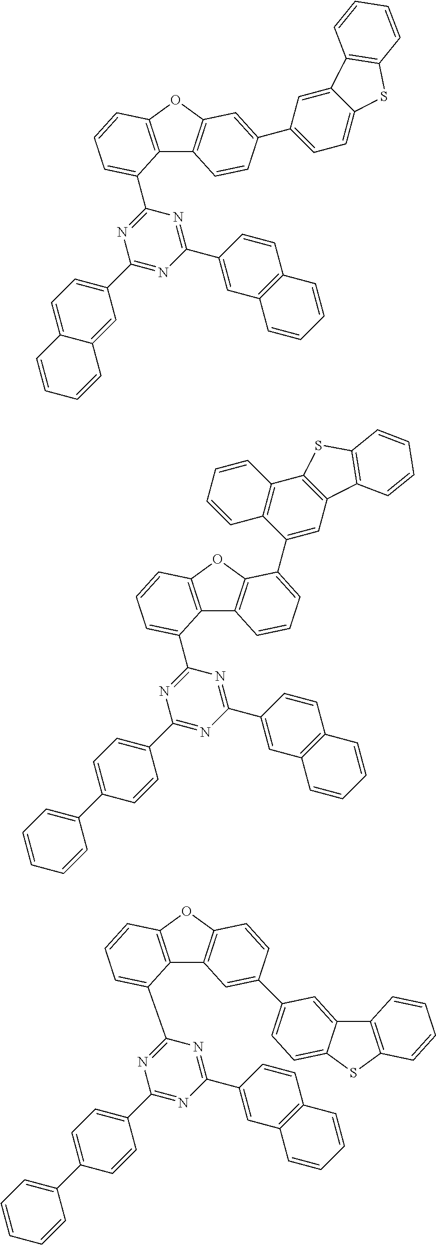 [SMILES: c1ccc2cc(-c3nc(-c4ccc5ccccc5c4)nc(-c4cccc5oc6cc(-c7ccc8sc9ccccc9c8c7)ccc6c45)n3)ccc2c1 ; c1ccc(-c2ccc(-c3nc(-c4ccc5ccccc5c4)nc(-c4cccc5oc6c(-c7cc8c9ccccc9sc8c8ccccc78)cccc6c45)n3)cc2)cc1 ; c1ccc(-c2ccc(-c3nc(-c4ccc5ccccc5c4)nc(-c4cccc5oc6ccc(-c7ccc8sc9ccccc9c8c7)cc6c45)n3)cc2)cc1]

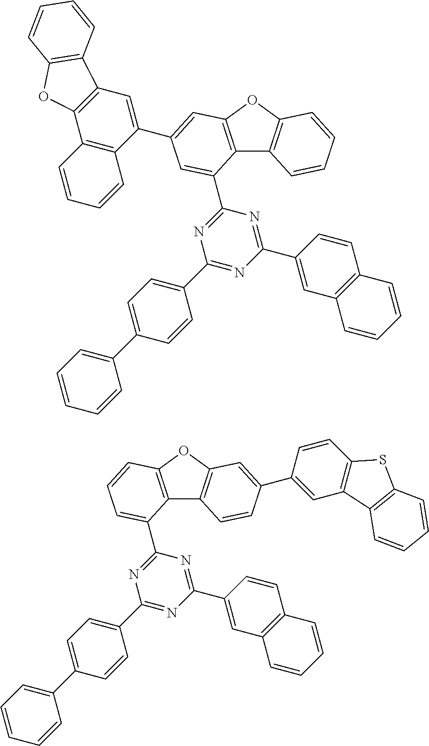 [SMILES: c1ccc(-c2ccc(-c3nc(-c4ccc5ccccc5c4)nc(-c4cc(-c5cc6c7ccccc7oc6c6ccccc56)cc5oc6ccccc6c45)n3)cc2)cc1 ; c1ccc(-c2ccc(-c3nc(-c4ccc5ccccc5c4)nc(-c4cccc5oc6cc(-c7ccc8sc9ccccc9c8c7)ccc6c45)n3)cc2)cc1]

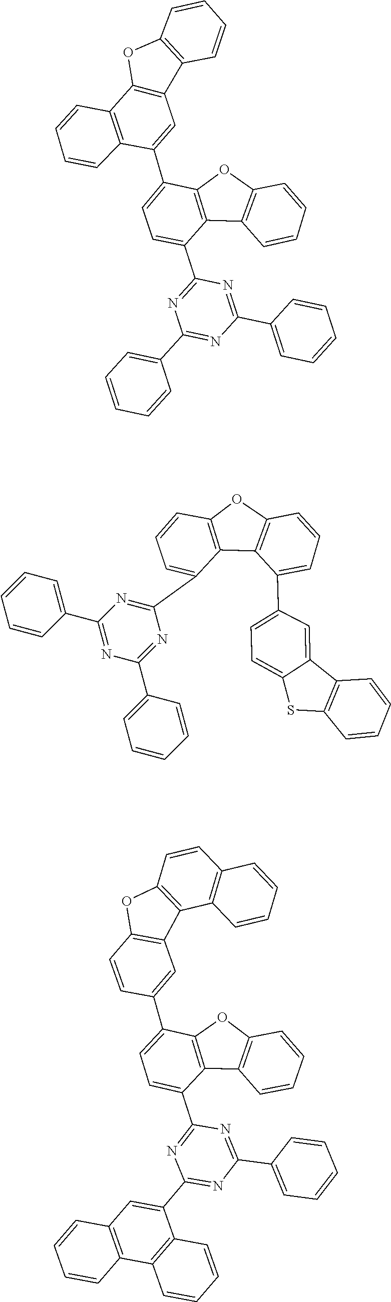 [SMILES: c1ccc(-c2nc(-c3ccccc3)nc(-c3ccc(-c4cc5c6ccccc6oc5c5ccccc45)c4oc5ccccc5c34)n2)cc1 ; c1ccc(-c2nc(-c3ccccc3)nc(-c3cccc4oc5cccc(-c6ccc7sc8ccccc8c7c6)c5c34)n2)cc1 ; c1ccc(-c2nc(-c3cc4ccccc4c4ccccc34)nc(-c3ccc(-c4ccc5oc6ccc7ccccc7c6c5c4)c4oc5ccccc5c34)n2)cc1]

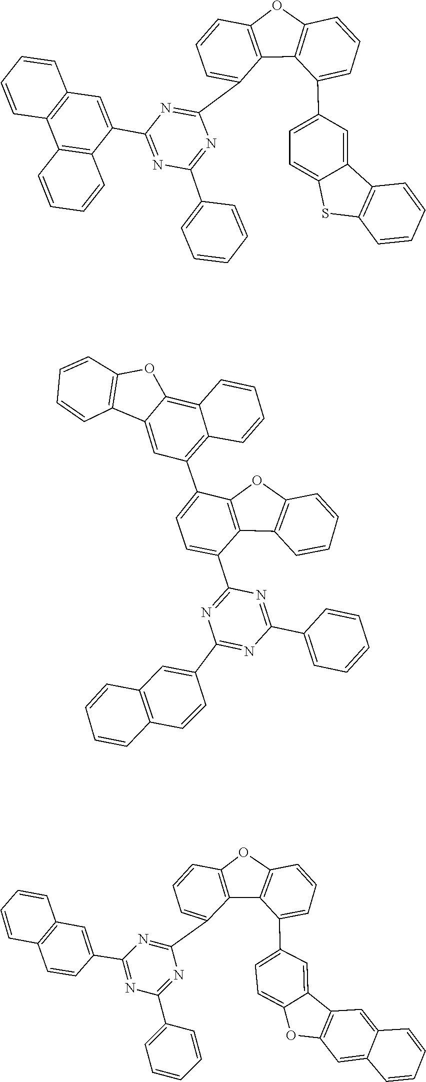 [SMILES: c1ccc(-c2nc(-c3cc4ccccc4c4ccccc34)nc(-c3cccc4oc5cccc(-c6ccc7sc8ccccc8c7c6)c5c34)n2)cc1 ; c1ccc(-c2nc(-c3ccc4ccccc4c3)nc(-c3ccc(-c4cc5c6ccccc6oc5c5ccccc45)c4oc5ccccc5c34)n2)cc1 ; c1ccc(-c2nc(-c3ccc4ccccc4c3)nc(-c3cccc4oc5cccc(-c6ccc7oc8cc9ccccc9cc8c7c6)c5c34)n2)cc1]

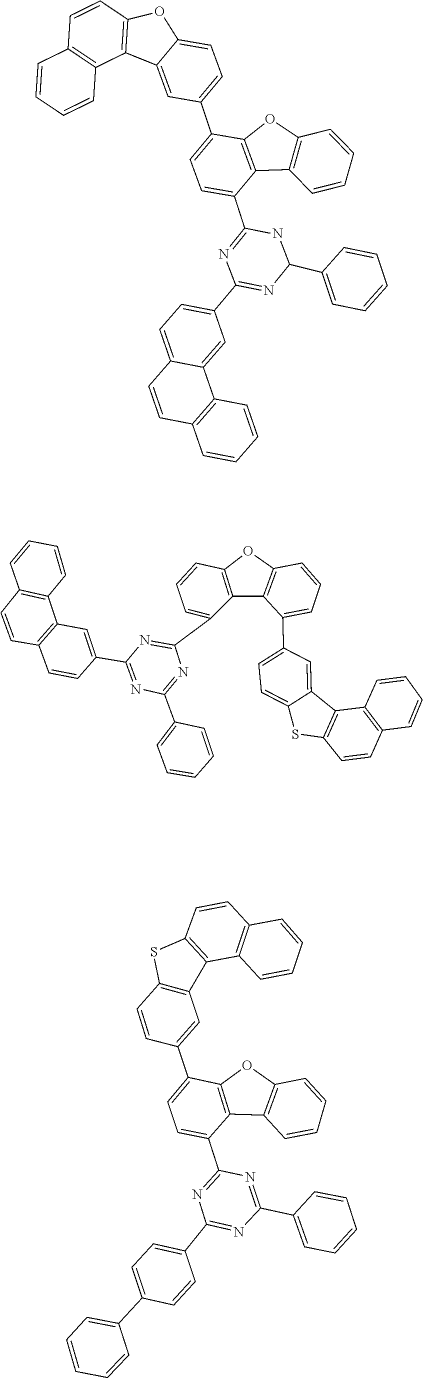

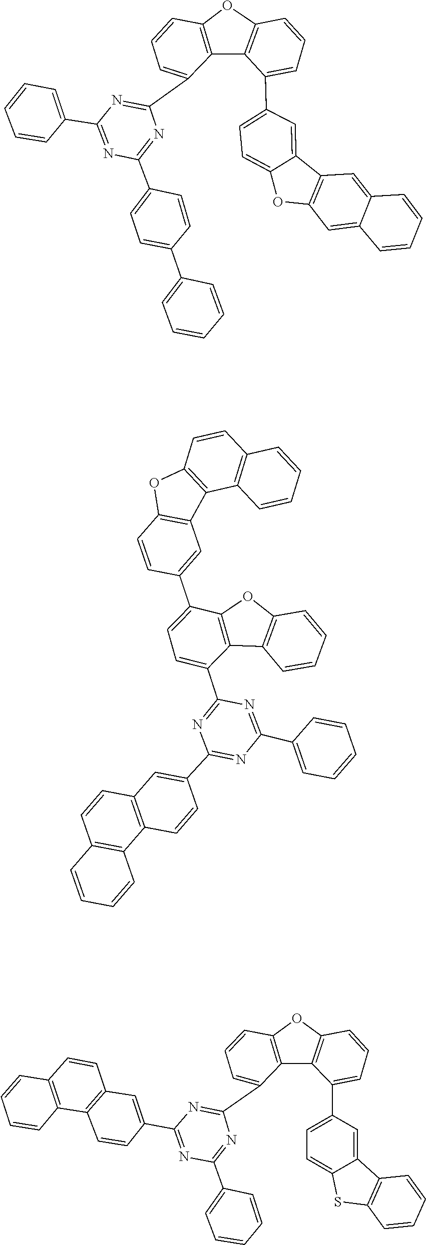 [SMILES: c1ccc(-c2ccc(-c3nc(-c4ccccc4)nc(-c4cccc5oc6cccc(-c7ccc8oc9cc%10ccccc%10cc9c8c7)c6c45)n3)cc2)cc1 ; c1ccc(-c2nc(-c3ccc4c(ccc5ccccc54)c3)nc(-c3ccc(-c4ccc5oc6ccc7ccccc7c6c5c4)c4oc5ccccc5c34)n2)cc1 ; c1ccc(-c2nc(-c3ccc4c(ccc5ccccc54)c3)nc(-c3cccc4oc5cccc(-c6ccc7sc8ccccc8c7c6)c5c34)n2)cc1]

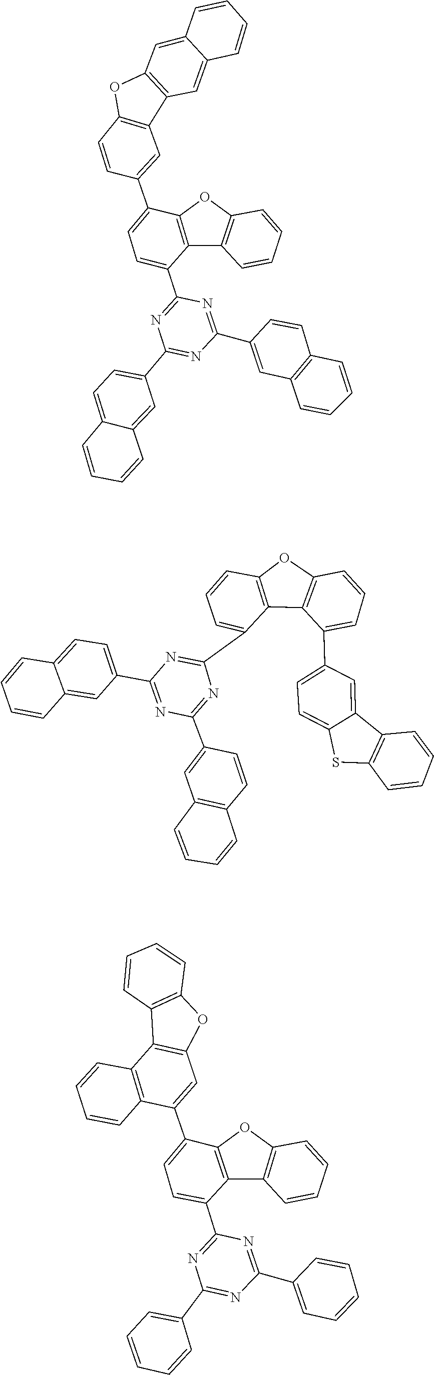 [SMILES: c1ccc2cc(-c3nc(-c4ccc5ccccc5c4)nc(-c4ccc(-c5ccc6oc7cc8ccccc8cc7c6c5)c5oc6ccccc6c45)n3)ccc2c1 ; c1ccc2cc(-c3nc(-c4ccc5ccccc5c4)nc(-c4cccc5oc6cccc(-c7ccc8sc9ccccc9c8c7)c6c45)n3)ccc2c1 ; c1ccc(-c2nc(-c3ccccc3)nc(-c3ccc(-c4cc5oc6ccccc6c5c5ccccc45)c4oc5ccccc5c34)n2)cc1]

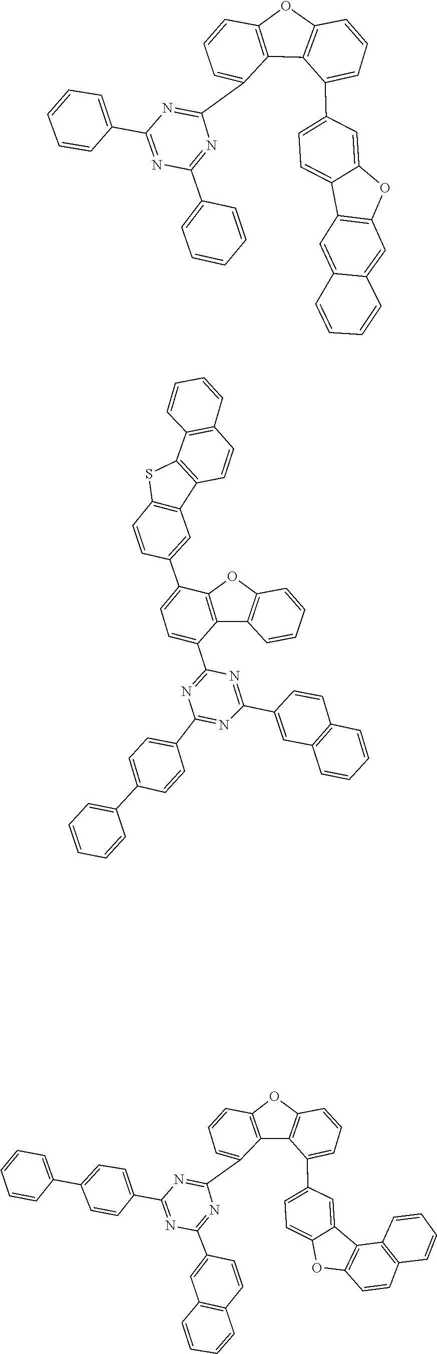 [SMILES: c1ccc(-c2nc(-c3ccccc3)nc(-c3cccc4oc5cccc(-c6ccc7c(c6)oc6cc8ccccc8cc67)c5c34)n2)cc1 ; c1ccc(-c2ccc(-c3nc(-c4ccc5ccccc5c4)nc(-c4ccc(-c5ccc6sc7c8ccccc8ccc7c6c5)c5oc6ccccc6c45)n3)cc2)cc1 ; c1ccc(-c2ccc(-c3nc(-c4ccc5ccccc5c4)nc(-c4cccc5oc6cccc(-c7ccc8oc9ccc%10ccccc%10c9c8c7)c6c45)n3)cc2)cc1]

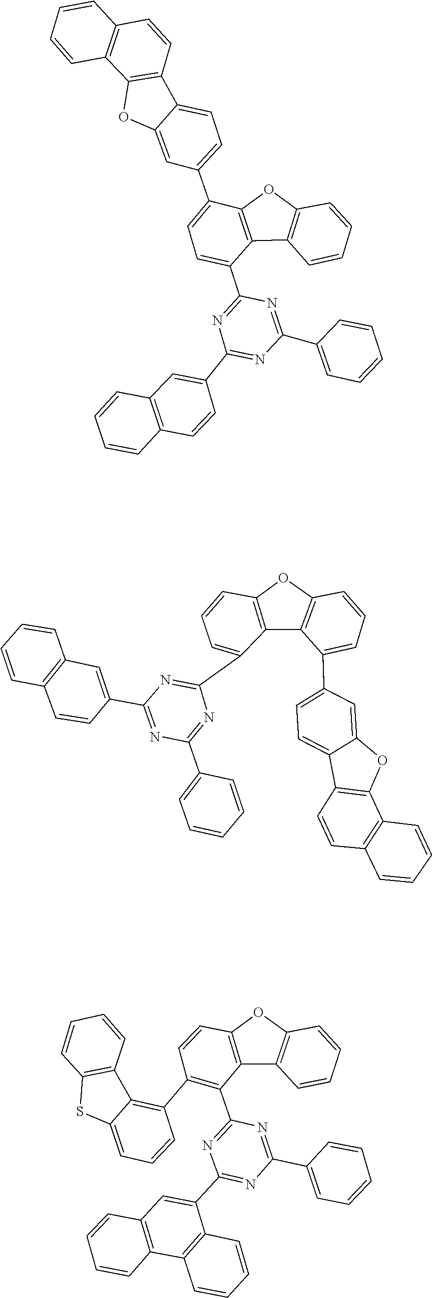 [SMILES: c1ccc(-c2nc(-c3ccc4ccccc4c3)nc(-c3ccc(-c4ccc5c(c4)oc4c6ccccc6ccc54)c4oc5ccccc5c34)n2)cc1 ; c1ccc(-c2nc(-c3ccc4ccccc4c3)nc(-c3cccc4oc5cccc(-c6ccc7c(c6)oc6c8ccccc8ccc76)c5c34)n2)cc1 ; c1ccc(-c2nc(-c3cc4ccccc4c4ccccc34)nc(-c3c(-c4cccc5sc6ccccc6c45)ccc4oc5ccccc5c34)n2)cc1]

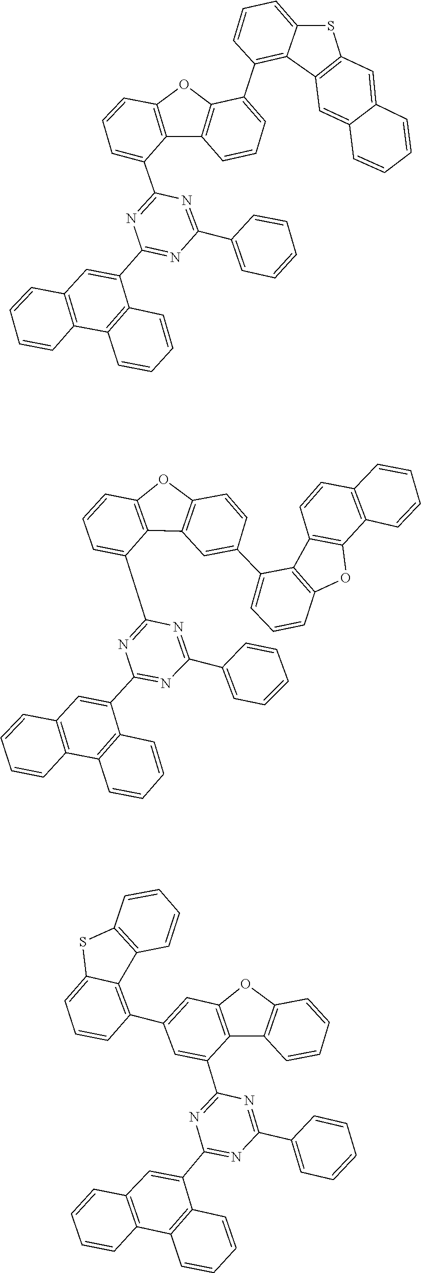 [SMILES: c1ccc(-c2nc(-c3cc4ccccc4c4ccccc34)nc(-c3cccc4oc5c(-c6cccc7sc8cc9ccccc9cc8c67)cccc5c34)n2)cc1 ; c1ccc(-c2nc(-c3cc4ccccc4c4ccccc34)nc(-c3cccc4oc5ccc(-c6cccc7oc8c9ccccc9ccc8c67)cc5c34)n2)cc1 ; c1ccc(-c2nc(-c3cc4ccccc4c4ccccc34)nc(-c3cc(-c4cccc5sc6ccccc6c45)cc4oc5ccccc5c34)n2)cc1]

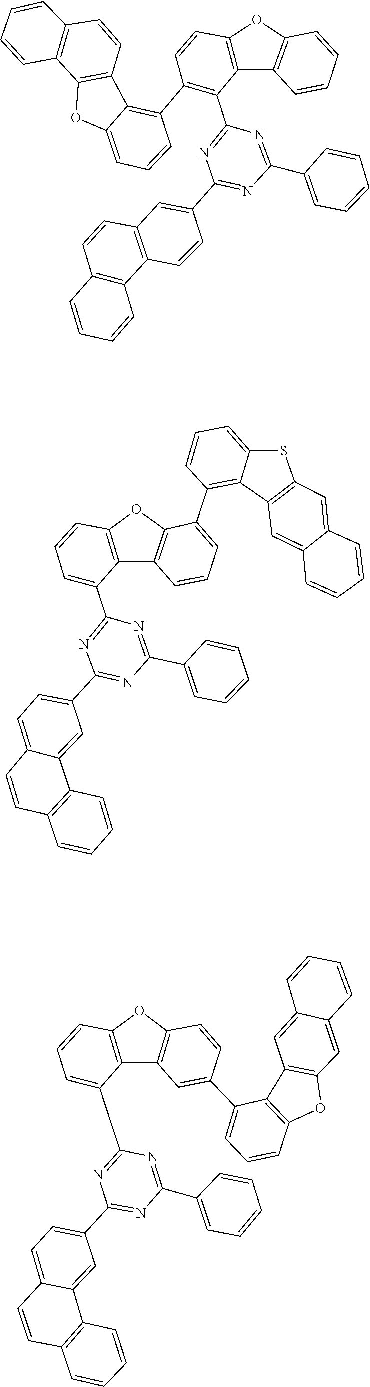 [SMILES: c1ccc(-c2nc(-c3ccc4c(ccc5ccccc54)c3)nc(-c3c(-c4cccc5oc6c7ccccc7ccc6c45)ccc4oc5ccccc5c34)n2)cc1 ; c1ccc(-c2nc(-c3ccc4ccc5ccccc5c4c3)nc(-c3cccc4oc5c(-c6cccc7sc8cc9ccccc9cc8c67)cccc5c34)n2)cc1 ; c1ccc(-c2nc(-c3ccc4ccc5ccccc5c4c3)nc(-c3cccc4oc5ccc(-c6cccc7oc8cc9ccccc9cc8c67)cc5c34)n2)cc1]

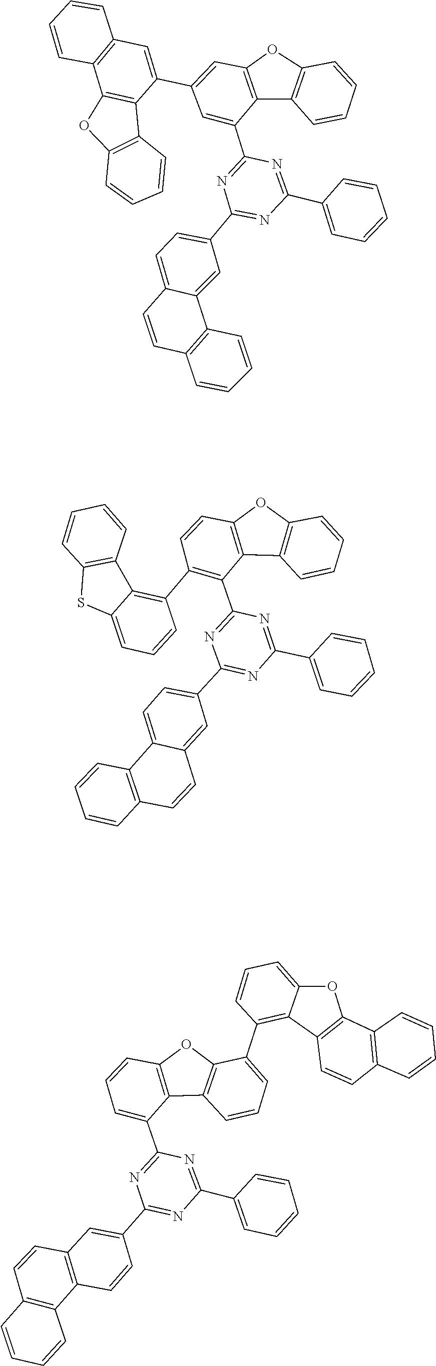 [SMILES: c1ccc(-c2nc(-c3ccc4ccc5ccccc5c4c3)nc(-c3cc(-c4cc5ccccc5c5oc6ccccc6c45)cc4oc5ccccc5c34)n2)cc1 ; c1ccc(-c2nc(-c3ccc4c(ccc5ccccc54)c3)nc(-c3c(-c4cccc5sc6ccccc6c45)ccc4oc5ccccc5c34)n2)cc1 ; c1ccc(-c2nc(-c3ccc4c(ccc5ccccc54)c3)nc(-c3cccc4oc5c(-c6cccc7oc8c9ccccc9ccc8c67)cccc5c34)n2)cc1]

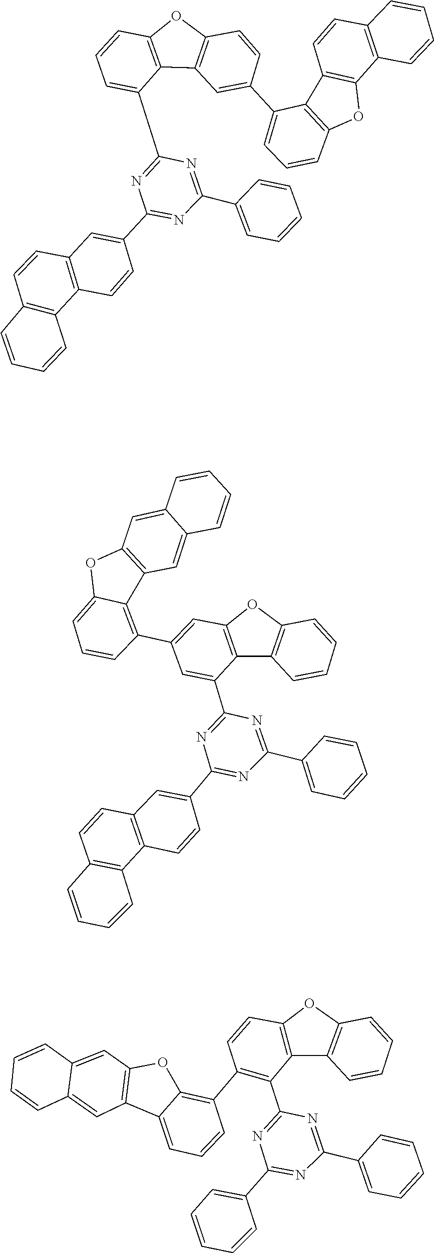 [SMILES: c1ccc(-c2nc(-c3ccc4c(ccc5ccccc54)c3)nc(-c3cccc4oc5ccc(-c6cccc7oc8c9ccccc9ccc8c67)cc5c34)n2)cc1 ; c1ccc(-c2nc(-c3ccc4c(ccc5ccccc54)c3)nc(-c3cc(-c4cccc5oc6cc7ccccc7cc6c45)cc4oc5ccccc5c34)n2)cc1 ; c1ccc(-c2nc(-c3ccccc3)nc(-c3c(-c4cccc5c4oc4cc6ccccc6cc45)ccc4oc5ccccc5c34)n2)cc1]

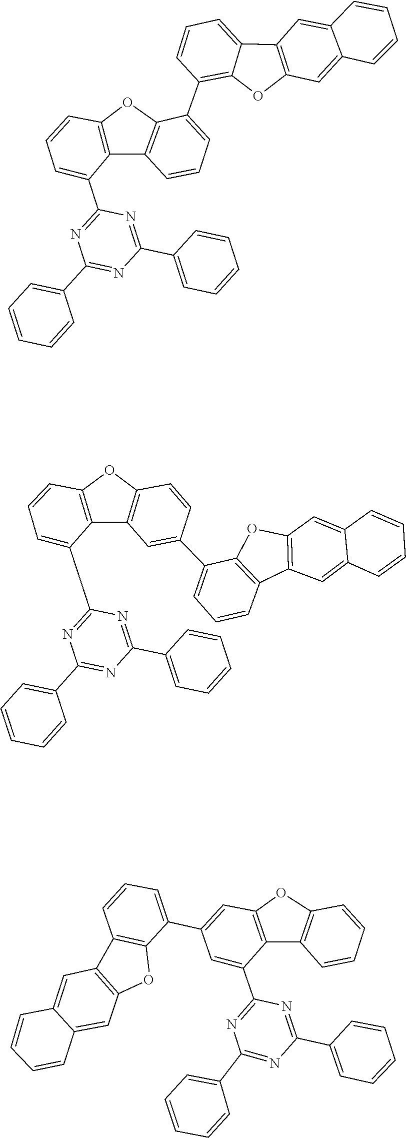 [SMILES: c1ccc(-c2nc(-c3ccccc3)nc(-c3cccc4oc5c(-c6cccc7c6oc6cc8ccccc8cc67)cccc5c34)n2)cc1 ; c1ccc(-c2nc(-c3ccccc3)nc(-c3cccc4oc5ccc(-c6cccc7c6oc6cc8ccccc8cc67)cc5c34)n2)cc1 ; c1ccc(-c2nc(-c3ccccc3)nc(-c3cc(-c4cccc5c4oc4cc6ccccc6cc45)cc4oc5ccccc5c34)n2)cc1]

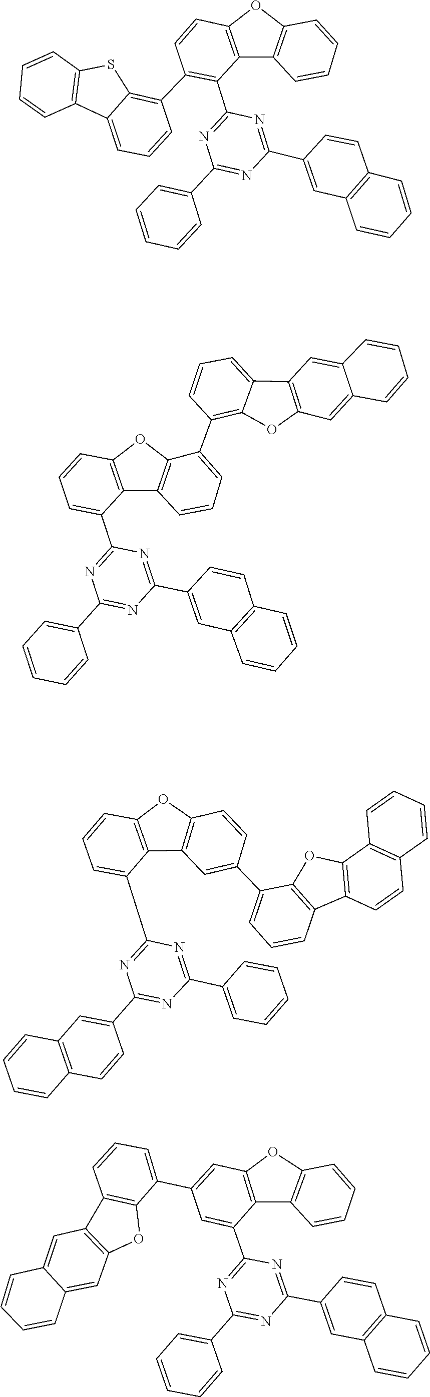 [SMILES: c1ccc(-c2nc(-c3ccc4ccccc4c3)nc(-c3c(-c4cccc5c4sc4ccccc45)ccc4oc5ccccc5c34)n2)cc1 ; c1ccc(-c2nc(-c3ccc4ccccc4c3)nc(-c3cccc4oc5c(-c6cccc7c6oc6cc8ccccc8cc67)cccc5c34)n2)cc1 ; c1ccc(-c2nc(-c3ccc4ccccc4c3)nc(-c3cccc4oc5ccc(-c6cccc7c6oc6c8ccccc8ccc76)cc5c34)n2)cc1 ; c1ccc(-c2nc(-c3ccc4ccccc4c3)nc(-c3cc(-c4cccc5c4oc4cc6ccccc6cc45)cc4oc5ccccc5c34)n2)cc1]

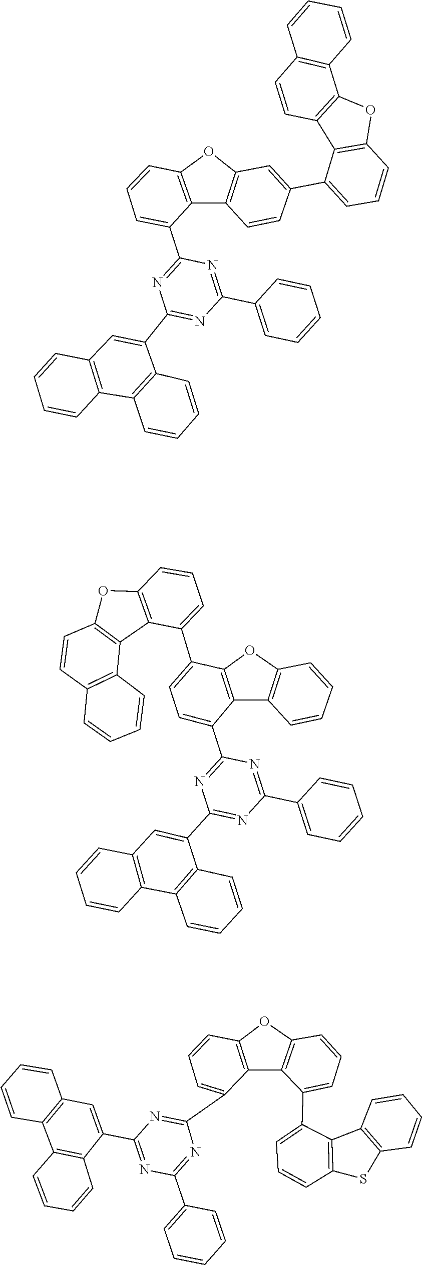 [SMILES: c1ccc(-c2nc(-c3cc4ccccc4c4ccccc34)nc(-c3cccc4oc5cc(-c6cccc7oc8c9ccccc9ccc8c67)ccc5c34)n2)cc1 ; c1ccc(-c2nc(-c3cc4ccccc4c4ccccc34)nc(-c3ccc(-c4cccc5oc6ccc7ccccc7c6c45)c4oc5ccccc5c34)n2)cc1 ; c1ccc(-c2nc(-c3cc4ccccc4c4ccccc34)nc(-c3cccc4oc5cccc(-c6cccc7sc8ccccc8c67)c5c34)n2)cc1]

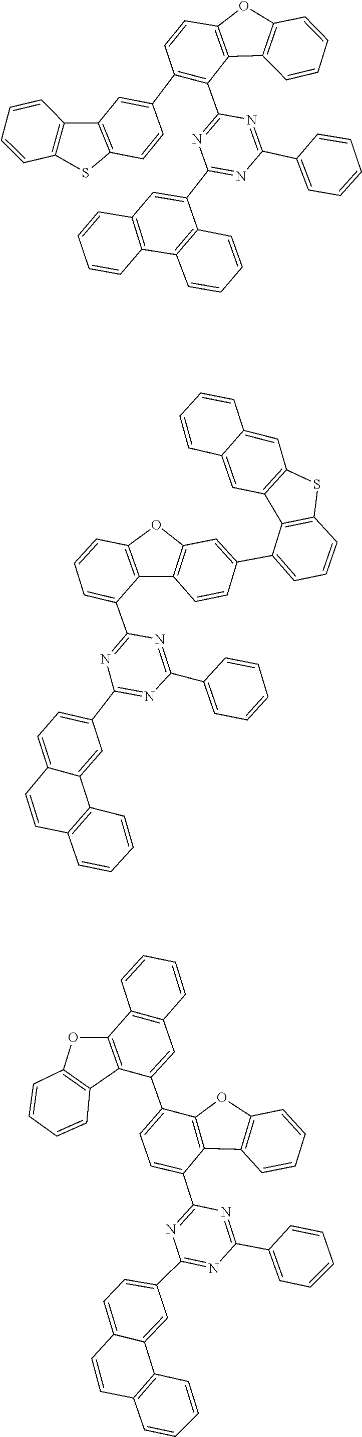 [SMILES: c1ccc(-c2nc(-c3cc4ccccc4c4ccccc34)nc(-c3c(-c4ccc5sc6ccccc6c5c4)ccc4oc5ccccc5c34)n2)cc1 ; c1ccc(-c2nc(-c3ccc4ccc5ccccc5c4c3)nc(-c3cccc4oc5cc(-c6cccc7sc8cc9ccccc9cc8c67)ccc5c34)n2)cc1 ; c1ccc(-c2nc(-c3ccc4ccc5ccccc5c4c3)nc(-c3ccc(-c4cc5ccccc5c5oc6ccccc6c45)c4oc5ccccc5c34)n2)cc1]

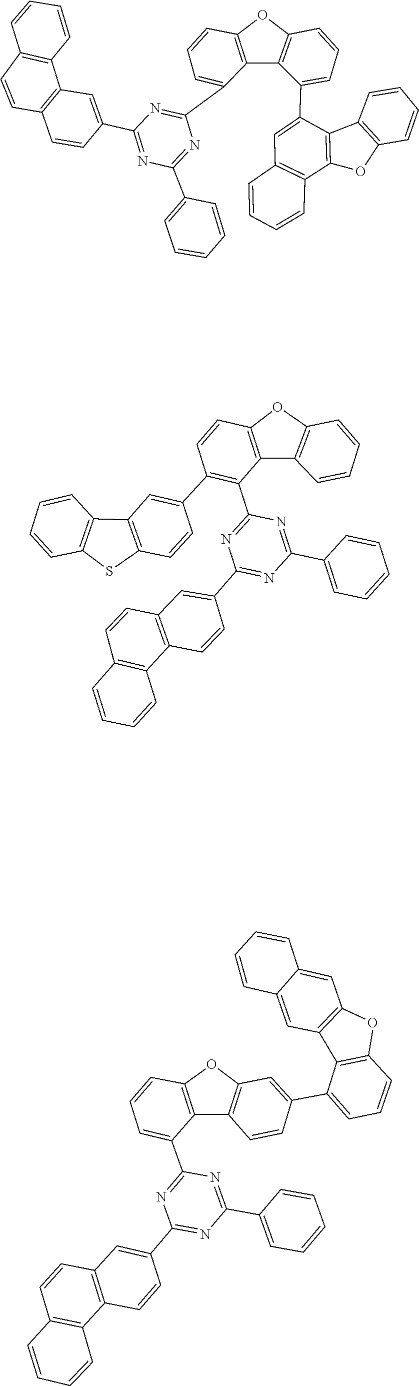 [SMILES: c1ccc(-c2nc(-c3ccc4ccc5ccccc5c4c3)nc(-c3cccc4oc5cccc(-c6cc7ccccc7c7oc8ccccc8c67)c5c34)n2)cc1 ; c1ccc(-c2nc(-c3ccc4c(ccc5ccccc54)c3)nc(-c3c(-c4ccc5sc6ccccc6c5c4)ccc4oc5ccccc5c34)n2)cc1 ; c1ccc(-c2nc(-c3ccc4c(ccc5ccccc54)c3)nc(-c3cccc4oc5cc(-c6cccc7oc8cc9ccccc9cc8c67)ccc5c34)n2)cc1]

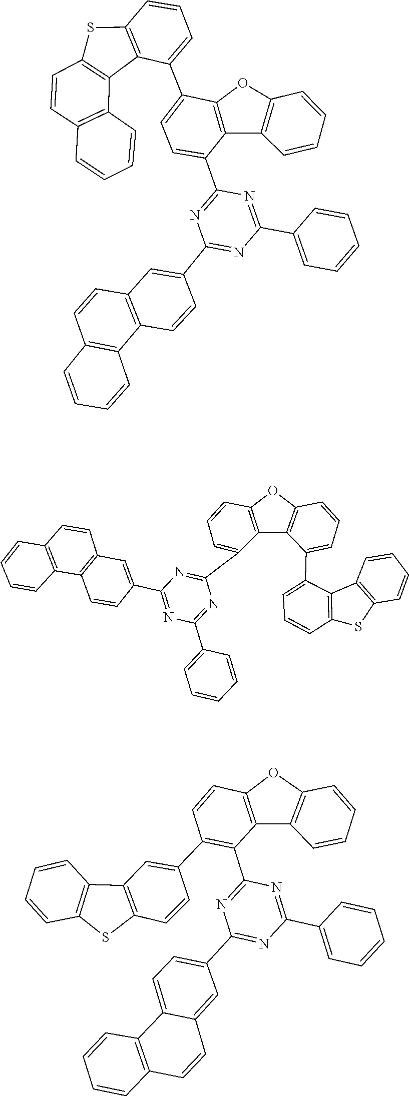 [SMILES: c1ccc(-c2nc(-c3ccc4c(ccc5ccccc54)c3)nc(-c3ccc(-c4cccc5sc6ccc7ccccc7c6c45)c4oc5ccccc5c34)n2)cc1 ; c1ccc(-c2nc(-c3ccc4c(ccc5ccccc54)c3)nc(-c3cccc4oc5cccc(-c6cccc7sc8ccccc8c67)c5c34)n2)cc1 ; c1ccc(-c2nc(-c3ccc4c(ccc5ccccc54)c3)nc(-c3c(-c4ccc5sc6ccccc6c5c4)ccc4oc5ccccc5c34)n2)cc1]

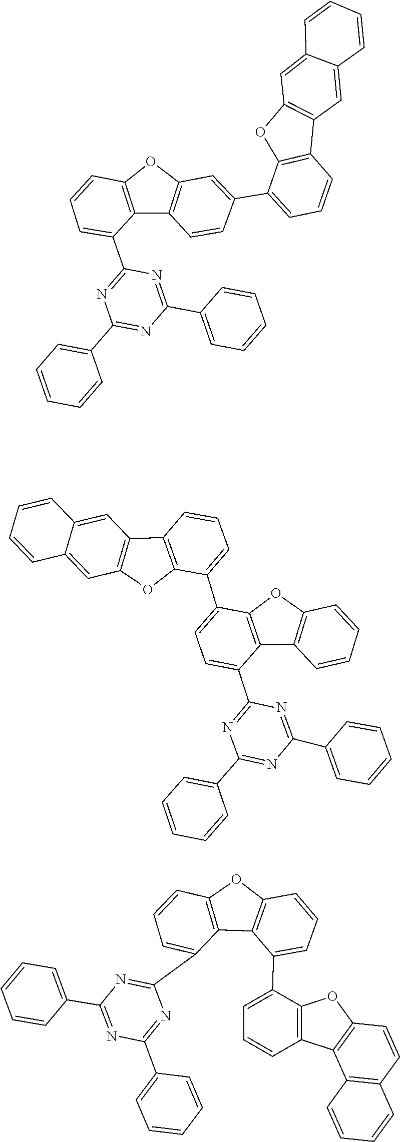 [SMILES: c1ccc(-c2nc(-c3ccccc3)nc(-c3cccc4oc5cc(-c6cccc7c6oc6cc8ccccc8cc67)ccc5c34)n2)cc1 ; c1ccc(-c2nc(-c3ccccc3)nc(-c3ccc(-c4cccc5c4oc4cc6ccccc6cc45)c4oc5ccccc5c34)n2)cc1 ; c1ccc(-c2nc(-c3ccccc3)nc(-c3cccc4oc5cccc(-c6cccc7c6oc6ccc8ccccc8c67)c5c34)n2)cc1]

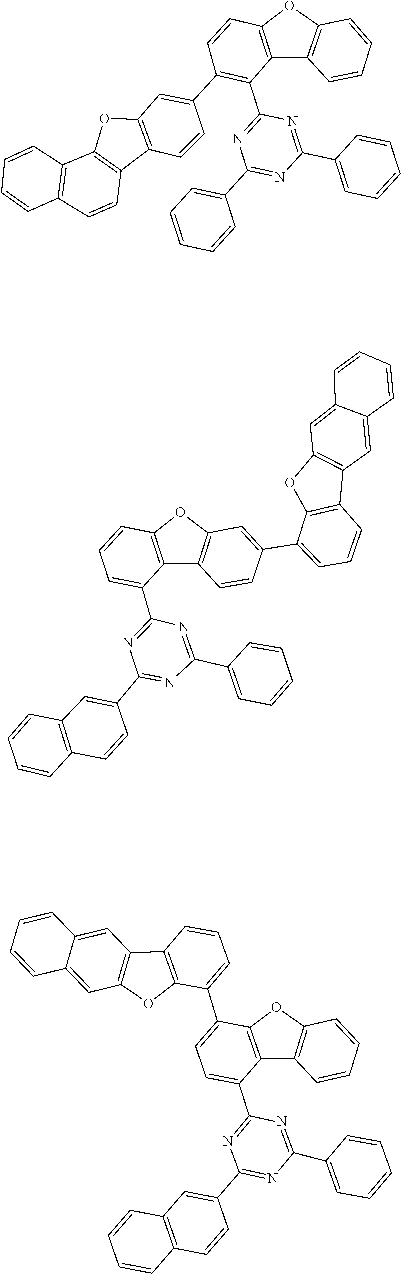 [SMILES: c1ccc(-c2nc(-c3ccccc3)nc(-c3c(-c4ccc5c(c4)oc4c6ccccc6ccc54)ccc4oc5ccccc5c34)n2)cc1 ; c1ccc(-c2nc(-c3ccc4ccccc4c3)nc(-c3cccc4oc5cc(-c6cccc7c6oc6cc8ccccc8cc67)ccc5c34)n2)cc1 ; c1ccc(-c2nc(-c3ccc4ccccc4c3)nc(-c3ccc(-c4cccc5c4oc4cc6ccccc6cc45)c4oc5ccccc5c34)n2)cc1]

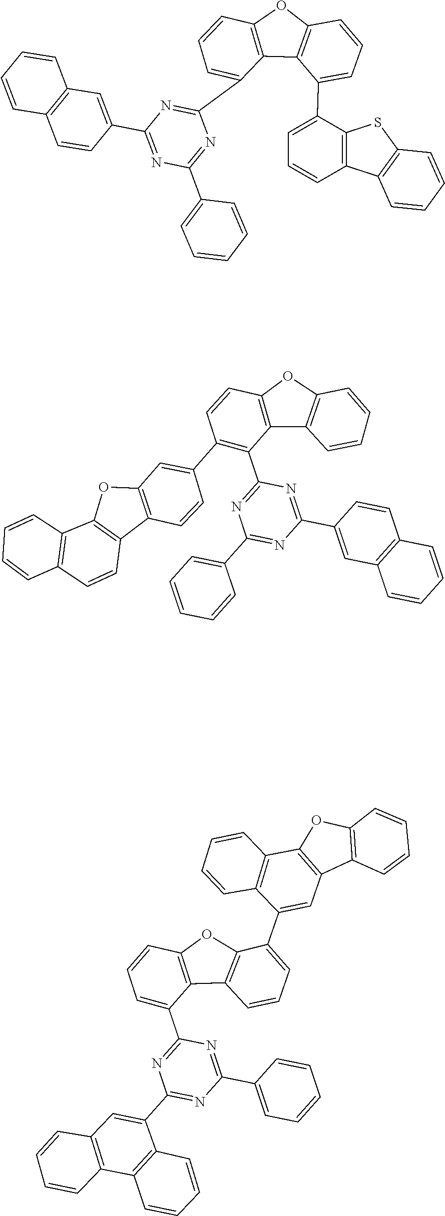 [SMILES: c1ccc(-c2nc(-c3ccc4ccccc4c3)nc(-c3cccc4oc5cccc(-c6cccc7c6sc6ccccc67)c5c34)n2)cc1 ; c1ccc(-c2nc(-c3ccc4ccccc4c3)nc(-c3c(-c4ccc5c(c4)oc4c6ccccc6ccc54)ccc4oc5ccccc5c34)n2)cc1 ; c1ccc(-c2nc(-c3cc4ccccc4c4ccccc34)nc(-c3cccc4oc5c(-c6cc7c8ccccc8oc7c7ccccc67)cccc5c34)n2)cc1]

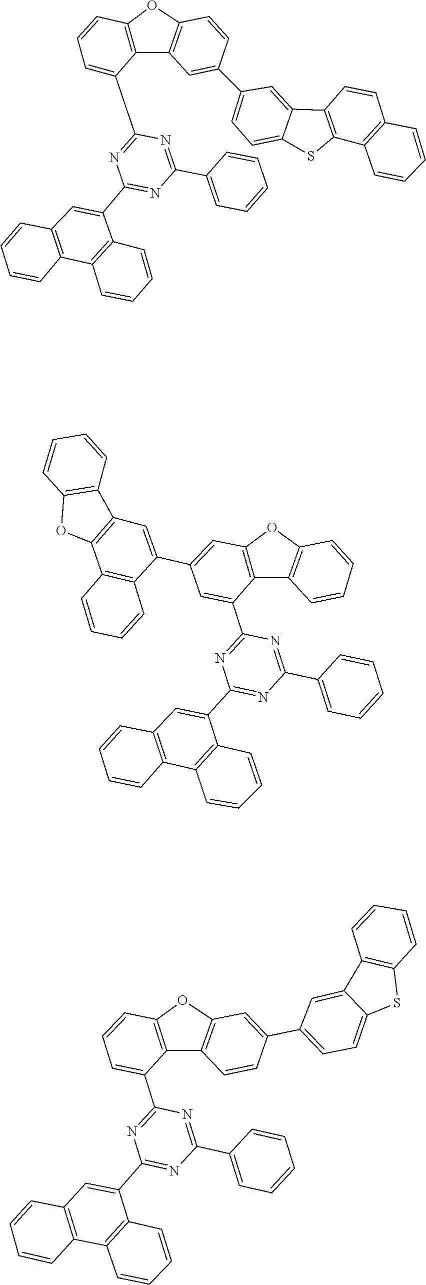 [SMILES: c1ccc(-c2nc(-c3cc4ccccc4c4ccccc34)nc(-c3cccc4oc5ccc(-c6ccc7sc8c9ccccc9ccc8c7c6)cc5c34)n2)cc1 ; c1ccc(-c2nc(-c3cc4ccccc4c4ccccc34)nc(-c3cc(-c4cc5c6ccccc6oc5c5ccccc45)cc4oc5ccccc5c34)n2)cc1 ; c1ccc(-c2nc(-c3cc4ccccc4c4ccccc34)nc(-c3cccc4oc5cc(-c6ccc7sc8ccccc8c7c6)ccc5c34)n2)cc1]

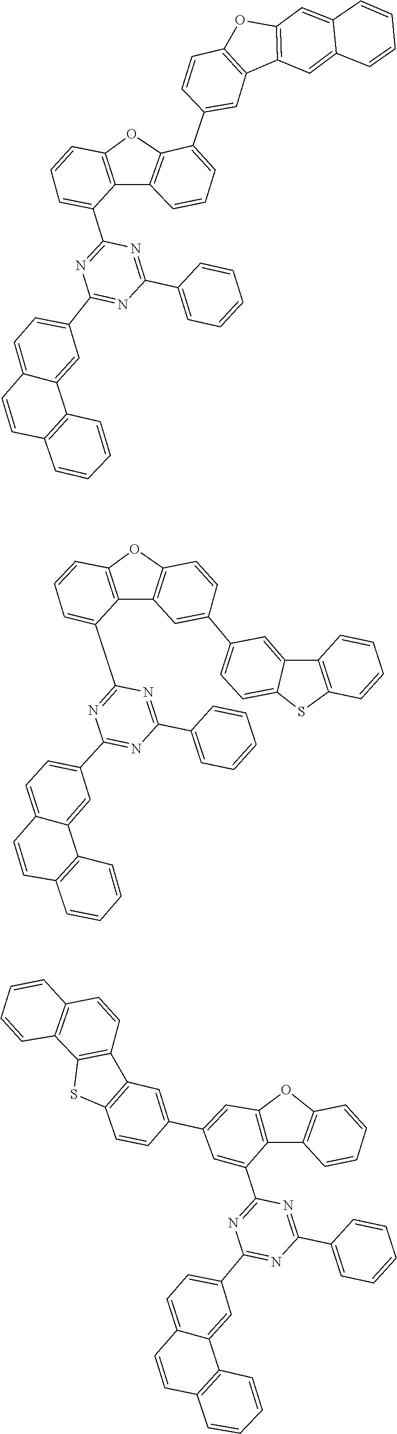 [SMILES: c1ccc(-c2nc(-c3ccc4ccc5ccccc5c4c3)nc(-c3cccc4oc5c(-c6ccc7oc8cc9ccccc9cc8c7c6)cccc5c34)n2)cc1 ; c1ccc(-c2nc(-c3ccc4ccc5ccccc5c4c3)nc(-c3cccc4oc5ccc(-c6ccc7sc8ccccc8c7c6)cc5c34)n2)cc1 ; c1ccc(-c2nc(-c3ccc4ccc5ccccc5c4c3)nc(-c3cc(-c4ccc5sc6c7ccccc7ccc6c5c4)cc4oc5ccccc5c34)n2)cc1]

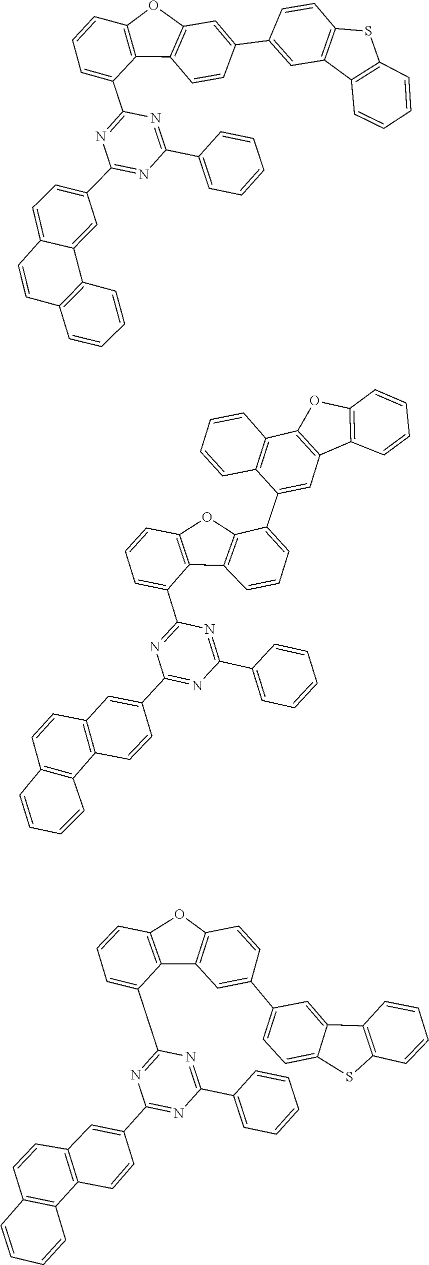 [SMILES: c1ccc(-c2nc(-c3ccc4ccc5ccccc5c4c3)nc(-c3cccc4oc5cc(-c6ccc7sc8ccccc8c7c6)ccc5c34)n2)cc1 ; c1ccc(-c2nc(-c3ccc4c(ccc5ccccc54)c3)nc(-c3cccc4oc5c(-c6cc7c8ccccc8oc7c7ccccc67)cccc5c34)n2)cc1 ; c1ccc(-c2nc(-c3ccc4c(ccc5ccccc54)c3)nc(-c3cccc4oc5ccc(-c6ccc7sc8ccccc8c7c6)cc5c34)n2)cc1]

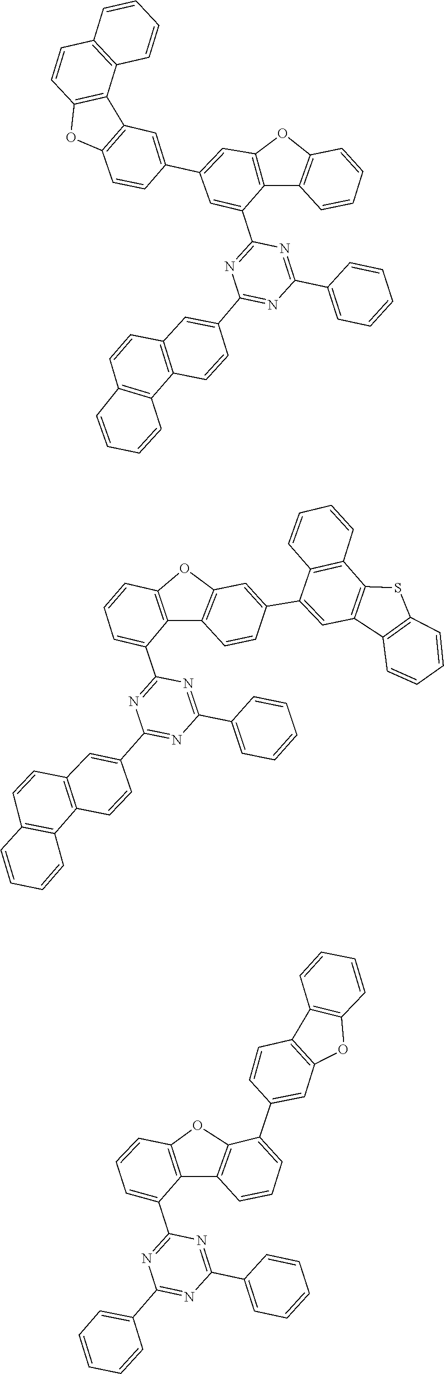 [SMILES: c1ccc(-c2nc(-c3ccc4c(ccc5ccccc54)c3)nc(-c3cc(-c4ccc5oc6ccc7ccccc7c6c5c4)cc4oc5ccccc5c34)n2)cc1 ; c1ccc(-c2nc(-c3ccc4c(ccc5ccccc54)c3)nc(-c3cccc4oc5cc(-c6cc7c8ccccc8sc7c7ccccc67)ccc5c34)n2)cc1 ; c1ccc(-c2nc(-c3ccccc3)nc(-c3cccc4oc5c(-c6ccc7c(c6)oc6ccccc67)cccc5c34)n2)cc1]

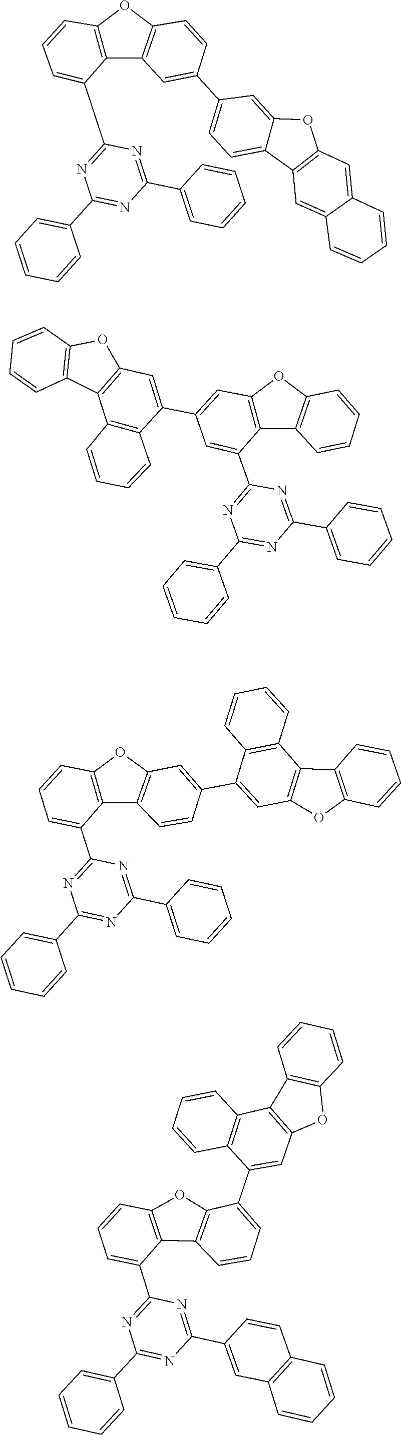 [SMILES: c1ccc(-c2nc(-c3ccccc3)nc(-c3cccc4oc5ccc(-c6ccc7c(c6)oc6cc8ccccc8cc67)cc5c34)n2)cc1 ; c1ccc(-c2nc(-c3ccccc3)nc(-c3cc(-c4cc5oc6ccccc6c5c5ccccc45)cc4oc5ccccc5c34)n2)cc1 ; c1ccc(-c2nc(-c3ccccc3)nc(-c3cccc4oc5cc(-c6cc7oc8ccccc8c7c7ccccc67)ccc5c34)n2)cc1 ; c1ccc(-c2nc(-c3ccc4ccccc4c3)nc(-c3cccc4oc5c(-c6cc7oc8ccccc8c7c7ccccc67)cccc5c34)n2)cc1]

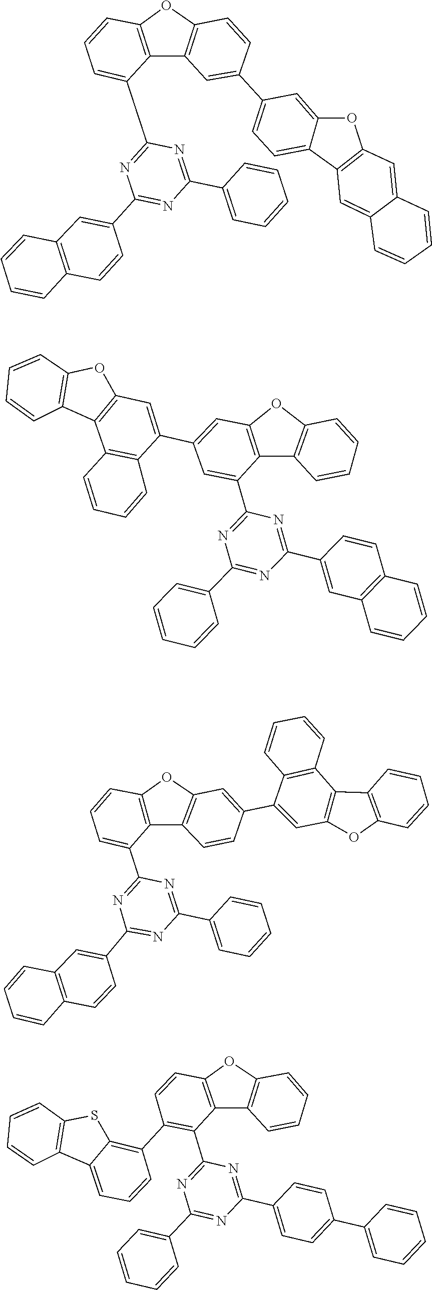 [SMILES: c1ccc(-c2nc(-c3ccc4ccccc4c3)nc(-c3cccc4oc5ccc(-c6ccc7c(c6)oc6cc8ccccc8cc67)cc5c34)n2)cc1 ; c1ccc(-c2nc(-c3ccc4ccccc4c3)nc(-c3cc(-c4cc5oc6ccccc6c5c5ccccc45)cc4oc5ccccc5c34)n2)cc1 ; c1ccc(-c2nc(-c3ccc4ccccc4c3)nc(-c3cccc4oc5cc(-c6cc7oc8ccccc8c7c7ccccc67)ccc5c34)n2)cc1 ; c1ccc(-c2ccc(-c3nc(-c4ccccc4)nc(-c4c(-c5cccc6c5sc5ccccc56)ccc5oc6ccccc6c45)n3)cc2)cc1]

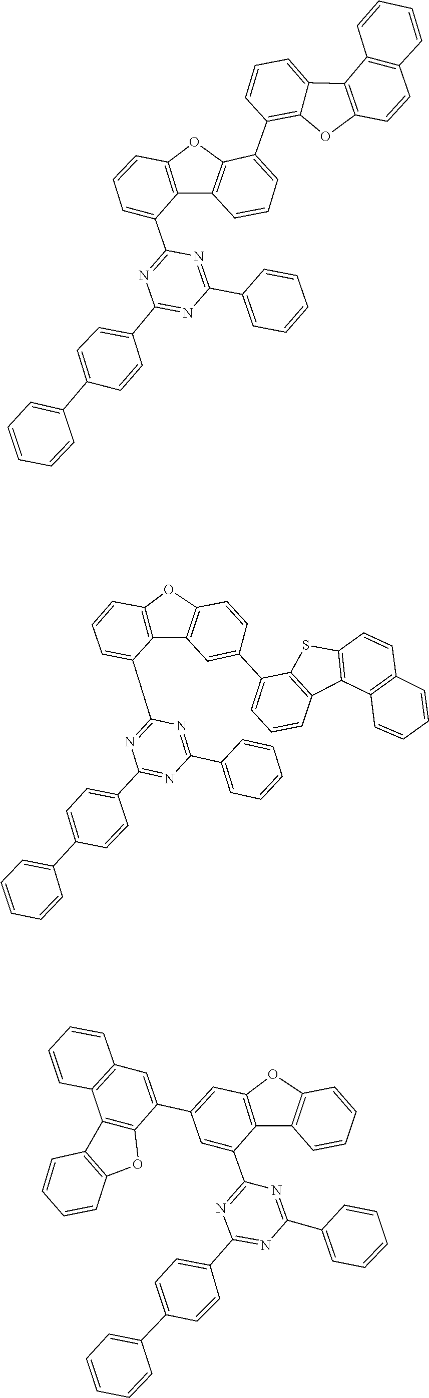 [SMILES: c1ccc(-c2ccc(-c3nc(-c4ccccc4)nc(-c4cccc5oc6c(-c7cccc8c7oc7ccc9ccccc9c78)cccc6c45)n3)cc2)cc1 ; c1ccc(-c2ccc(-c3nc(-c4ccccc4)nc(-c4cccc5oc6ccc(-c7cccc8c7sc7ccc9ccccc9c78)cc6c45)n3)cc2)cc1 ; c1ccc(-c2ccc(-c3nc(-c4ccccc4)nc(-c4cc(-c5cc6ccccc6c6c5oc5ccccc56)cc5oc6ccccc6c45)n3)cc2)cc1]

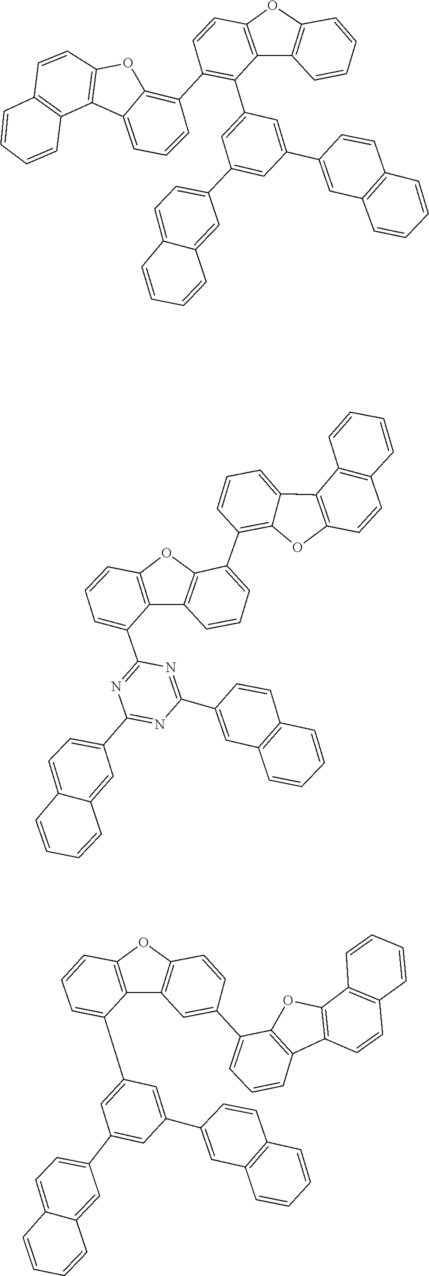 [SMILES: c1ccc2cc(-c3cc(-c4ccc5ccccc5c4)cc(-c4c(-c5cccc6c5oc5ccc7ccccc7c56)ccc5oc6ccccc6c45)c3)ccc2c1 ; c1ccc2cc(-c3nc(-c4ccc5ccccc5c4)nc(-c4cccc5oc6c(-c7cccc8c7oc7ccc9ccccc9c78)cccc6c45)n3)ccc2c1 ; c1ccc2cc(-c3cc(-c4ccc5ccccc5c4)cc(-c4cccc5oc6ccc(-c7cccc8c7oc7c9ccccc9ccc87)cc6c45)c3)ccc2c1]

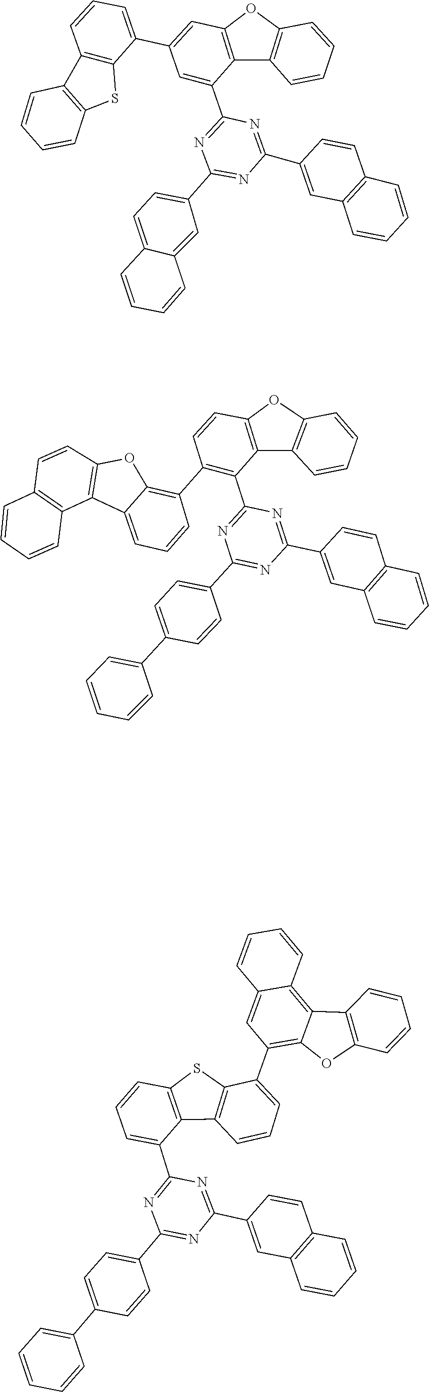 [SMILES: c1ccc2cc(-c3nc(-c4ccc5ccccc5c4)nc(-c4cc(-c5cccc6c5sc5ccccc56)cc5oc6ccccc6c45)n3)ccc2c1 ; c1ccc(-c2ccc(-c3nc(-c4ccc5ccccc5c4)nc(-c4c(-c5cccc6c5oc5ccc7ccccc7c56)ccc5oc6ccccc6c45)n3)cc2)cc1 ; c1ccc(-c2ccc(-c3nc(-c4ccc5ccccc5c4)nc(-c4cccc5sc6c(-c7cc8ccccc8c8c7oc7ccccc78)cccc6c45)n3)cc2)cc1]

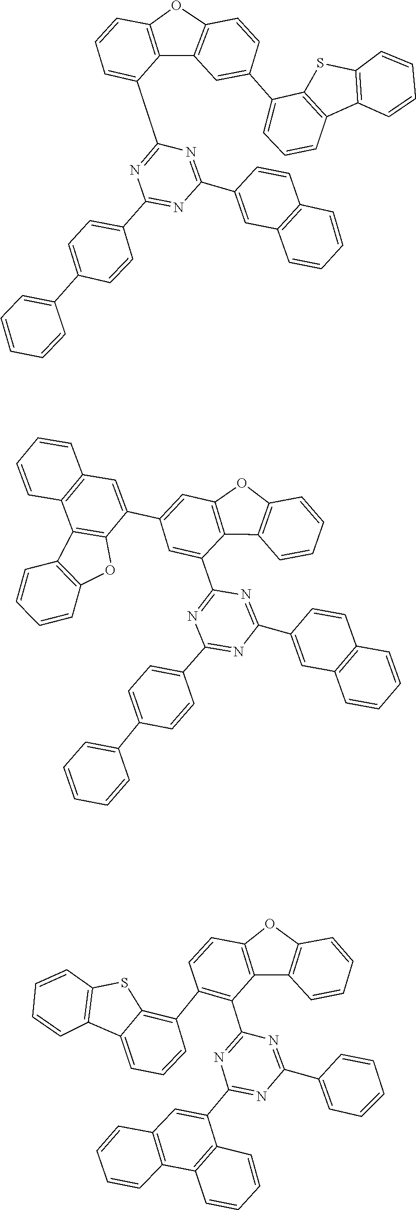 [SMILES: c1ccc(-c2ccc(-c3nc(-c4ccc5ccccc5c4)nc(-c4cccc5oc6ccc(-c7cccc8c7sc7ccccc78)cc6c45)n3)cc2)cc1 ; c1ccc(-c2ccc(-c3nc(-c4ccc5ccccc5c4)nc(-c4cc(-c5cc6ccccc6c6c5oc5ccccc56)cc5oc6ccccc6c45)n3)cc2)cc1 ; c1ccc(-c2nc(-c3cc4ccccc4c4ccccc34)nc(-c3c(-c4cccc5c4sc4ccccc45)ccc4oc5ccccc5c34)n2)cc1]

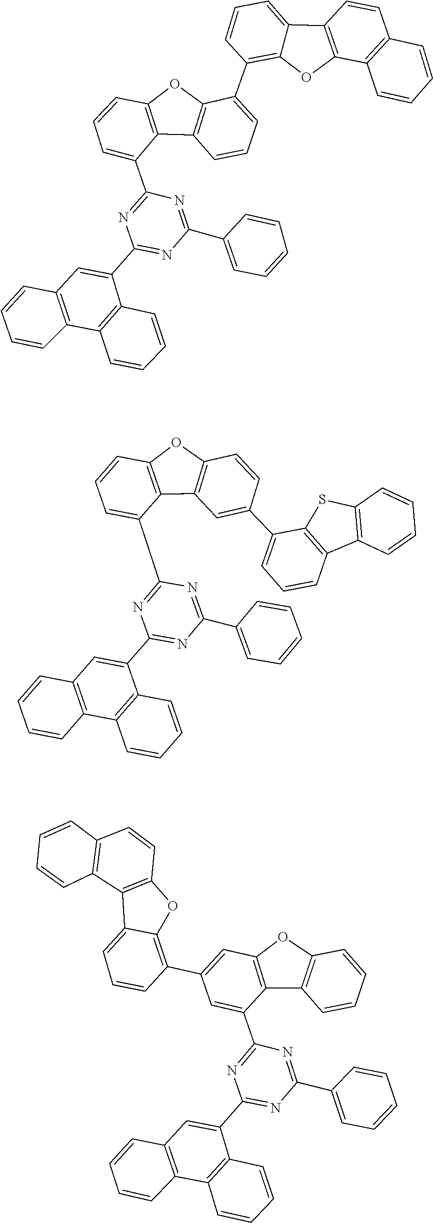 [SMILES: c1ccc(-c2nc(-c3cc4ccccc4c4ccccc34)nc(-c3cccc4oc5c(-c6cccc7c6oc6c8ccccc8ccc76)cccc5c34)n2)cc1 ; c1ccc(-c2nc(-c3cc4ccccc4c4ccccc34)nc(-c3cccc4oc5ccc(-c6cccc7c6sc6ccccc67)cc5c34)n2)cc1 ; c1ccc(-c2nc(-c3cc4ccccc4c4ccccc34)nc(-c3cc(-c4cccc5c4oc4ccc6ccccc6c45)cc4oc5ccccc5c34)n2)cc1]

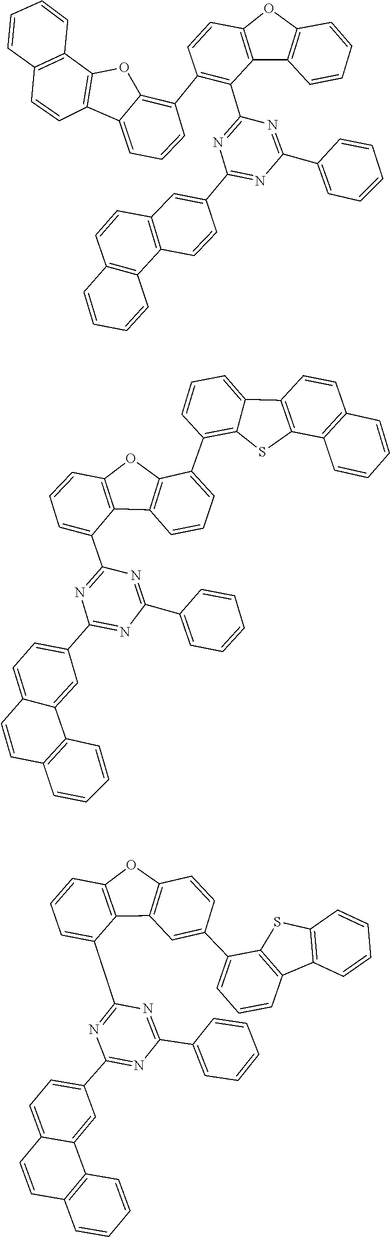 [SMILES: c1ccc(-c2nc(-c3ccc4c(ccc5ccccc54)c3)nc(-c3c(-c4cccc5c4oc4c6ccccc6ccc54)ccc4oc5ccccc5c34)n2)cc1 ; c1ccc(-c2nc(-c3ccc4ccc5ccccc5c4c3)nc(-c3cccc4oc5c(-c6cccc7c6sc6c8ccccc8ccc76)cccc5c34)n2)cc1 ; c1ccc(-c2nc(-c3ccc4ccc5ccccc5c4c3)nc(-c3cccc4oc5ccc(-c6cccc7c6sc6ccccc67)cc5c34)n2)cc1]

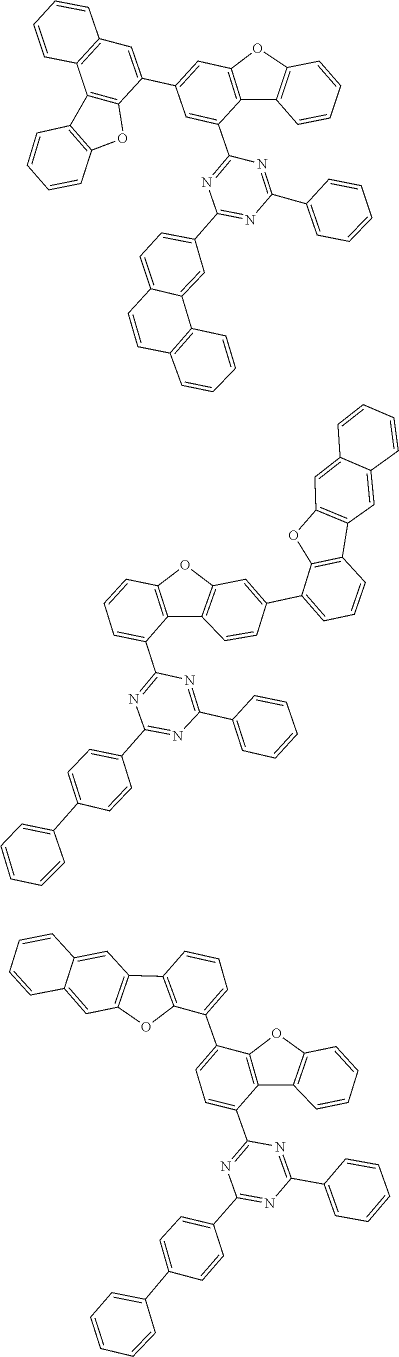 [SMILES: c1ccc(-c2nc(-c3ccc4ccc5ccccc5c4c3)nc(-c3cc(-c4cc5ccccc5c5c4oc4ccccc45)cc4oc5ccccc5c34)n2)cc1 ; c1ccc(-c2ccc(-c3nc(-c4ccccc4)nc(-c4cccc5oc6cc(-c7cccc8c7oc7cc9ccccc9cc78)ccc6c45)n3)cc2)cc1 ; c1ccc(-c2ccc(-c3nc(-c4ccccc4)nc(-c4ccc(-c5cccc6c5oc5cc7ccccc7cc56)c5oc6ccccc6c45)n3)cc2)cc1]

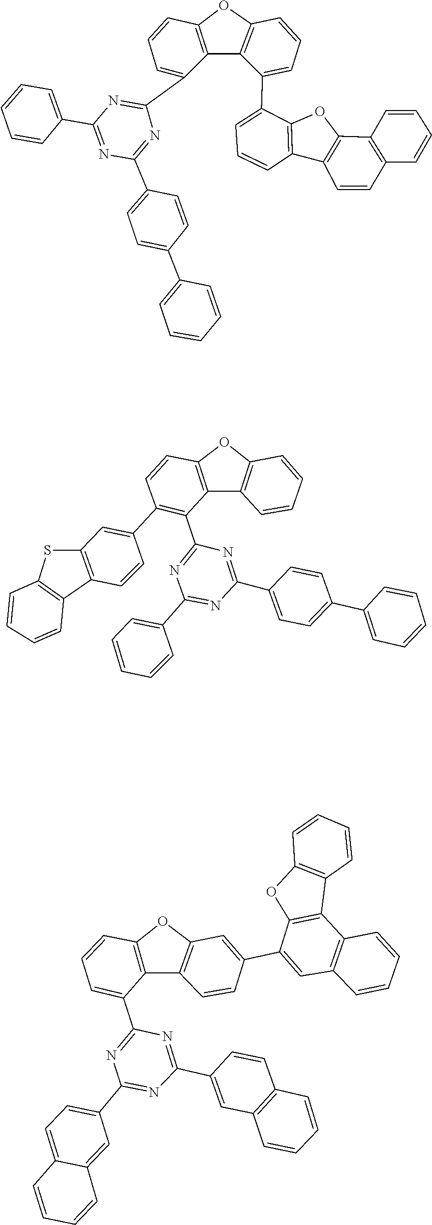 [SMILES: c1ccc(-c2ccc(-c3nc(-c4ccccc4)nc(-c4cccc5oc6cccc(-c7cccc8c7oc7c9ccccc9ccc87)c6c45)n3)cc2)cc1 ; c1ccc(-c2ccc(-c3nc(-c4ccccc4)nc(-c4c(-c5ccc6c(c5)sc5ccccc56)ccc5oc6ccccc6c45)n3)cc2)cc1 ; c1ccc2cc(-c3nc(-c4ccc5ccccc5c4)nc(-c4cccc5oc6cc(-c7cc8ccccc8c8c7oc7ccccc78)ccc6c45)n3)ccc2c1]

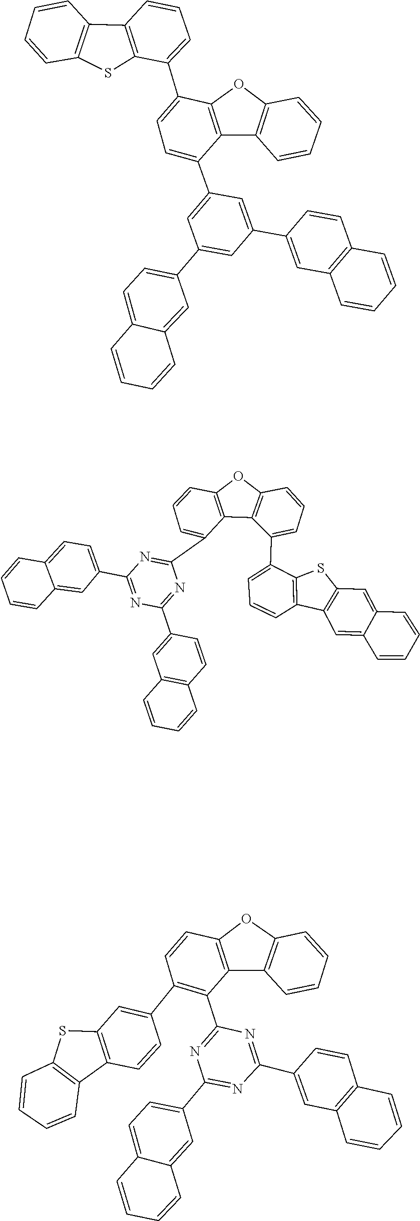 [SMILES: c1ccc2cc(-c3cc(-c4ccc5ccccc5c4)cc(-c4ccc(-c5cccc6c5sc5ccccc56)c5oc6ccccc6c45)c3)ccc2c1 ; c1ccc2cc(-c3nc(-c4ccc5ccccc5c4)nc(-c4cccc5oc6cccc(-c7cccc8c7sc7cc9ccccc9cc78)c6c45)n3)ccc2c1 ; c1ccc2cc(-c3nc(-c4ccc5ccccc5c4)nc(-c4c(-c5ccc6c(c5)sc5ccccc56)ccc5oc6ccccc6c45)n3)ccc2c1]

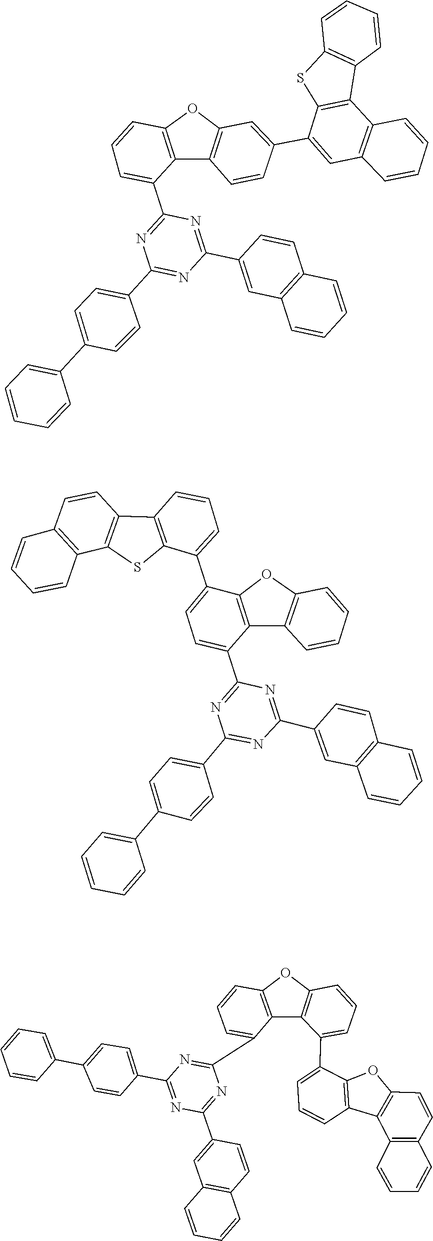 [SMILES: c1ccc(-c2ccc(-c3nc(-c4ccc5ccccc5c4)nc(-c4cccc5oc6cc(-c7cc8ccccc8c8c7sc7ccccc78)ccc6c45)n3)cc2)cc1 ; c1ccc(-c2ccc(-c3nc(-c4ccc5ccccc5c4)nc(-c4ccc(-c5cccc6c5sc5c7ccccc7ccc65)c5oc6ccccc6c45)n3)cc2)cc1 ; c1ccc(-c2ccc(-c3nc(-c4ccc5ccccc5c4)nc(-c4cccc5oc6cccc(-c7cccc8c7oc7ccc9ccccc9c78)c6c45)n3)cc2)cc1]

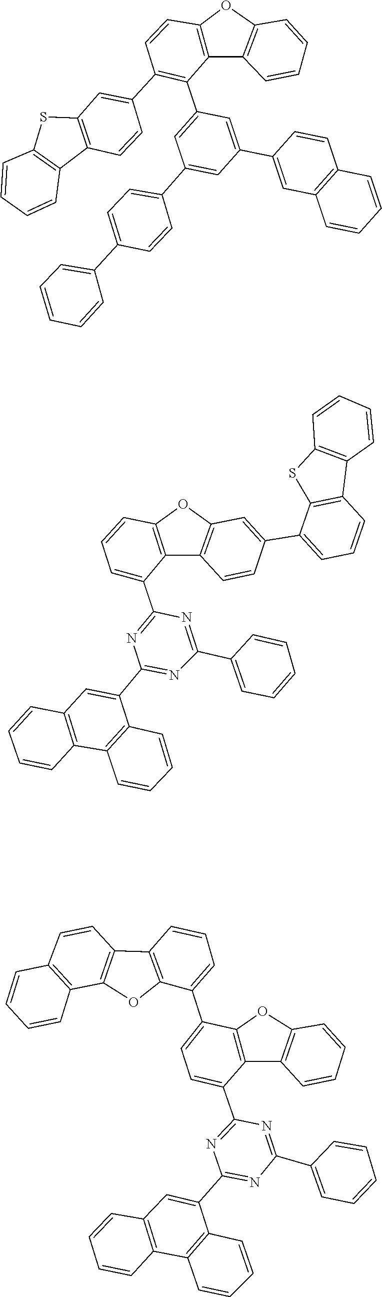 [SMILES: c1ccc(-c2ccc(-c3cc(-c4ccc5ccccc5c4)cc(-c4c(-c5ccc6c(c5)sc5ccccc56)ccc5oc6ccccc6c45)c3)cc2)cc1 ; c1ccc(-c2nc(-c3cc4ccccc4c4ccccc34)nc(-c3cccc4oc5cc(-c6cccc7c6sc6ccccc67)ccc5c34)n2)cc1 ; c1ccc(-c2nc(-c3cc4ccccc4c4ccccc34)nc(-c3ccc(-c4cccc5c4oc4c6ccccc6ccc54)c4oc5ccccc5c34)n2)cc1]

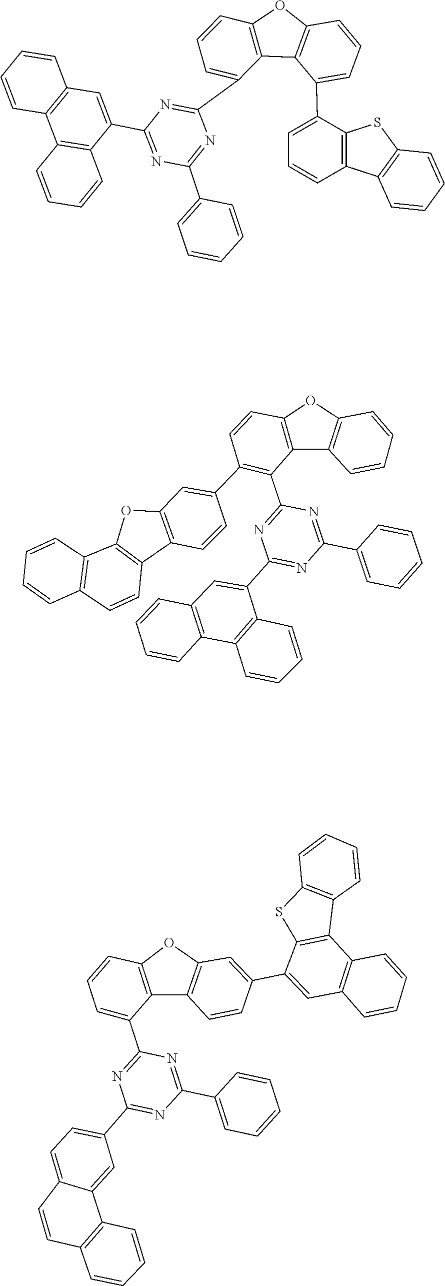 [SMILES: c1ccc(-c2nc(-c3cc4ccccc4c4ccccc34)nc(-c3cccc4oc5cccc(-c6cccc7c6sc6ccccc67)c5c34)n2)cc1 ; c1ccc(-c2nc(-c3cc4ccccc4c4ccccc34)nc(-c3c(-c4ccc5c(c4)oc4c6ccccc6ccc54)ccc4oc5ccccc5c34)n2)cc1 ; c1ccc(-c2nc(-c3ccc4ccc5ccccc5c4c3)nc(-c3cccc4oc5cc(-c6cc7ccccc7c7c6sc6ccccc67)ccc5c34)n2)cc1]

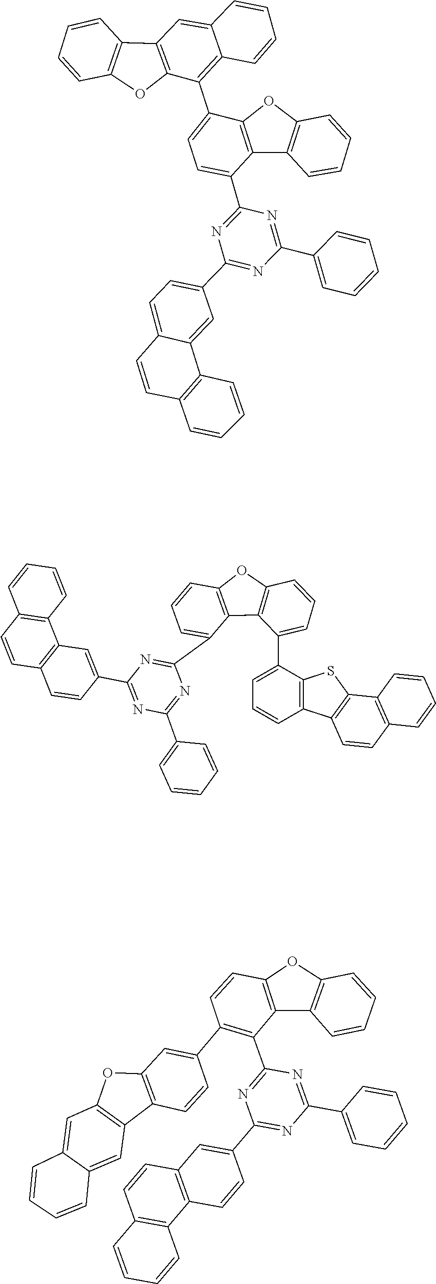 [SMILES: c1ccc(-c2nc(-c3ccc4ccc5ccccc5c4c3)nc(-c3ccc(-c4c5ccccc5cc5c4oc4ccccc45)c4oc5ccccc5c34)n2)cc1 ; c1ccc(-c2nc(-c3ccc4ccc5ccccc5c4c3)nc(-c3cccc4oc5cccc(-c6cccc7c6sc6c8ccccc8ccc76)c5c34)n2)cc1 ; c1ccc(-c2nc(-c3ccc4c(ccc5ccccc54)c3)nc(-c3c(-c4ccc5c(c4)oc4cc6ccccc6cc45)ccc4oc5ccccc5c34)n2)cc1]

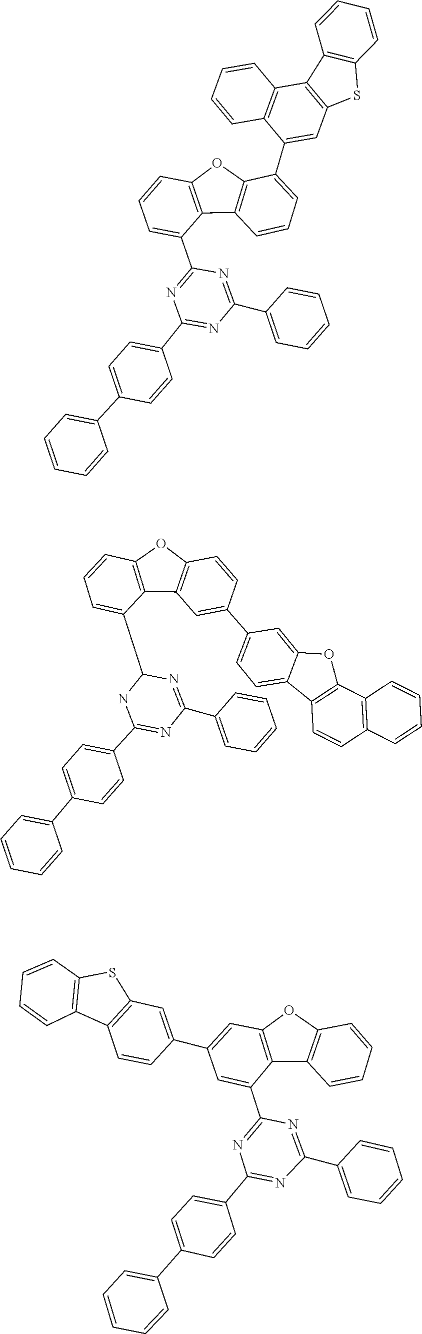

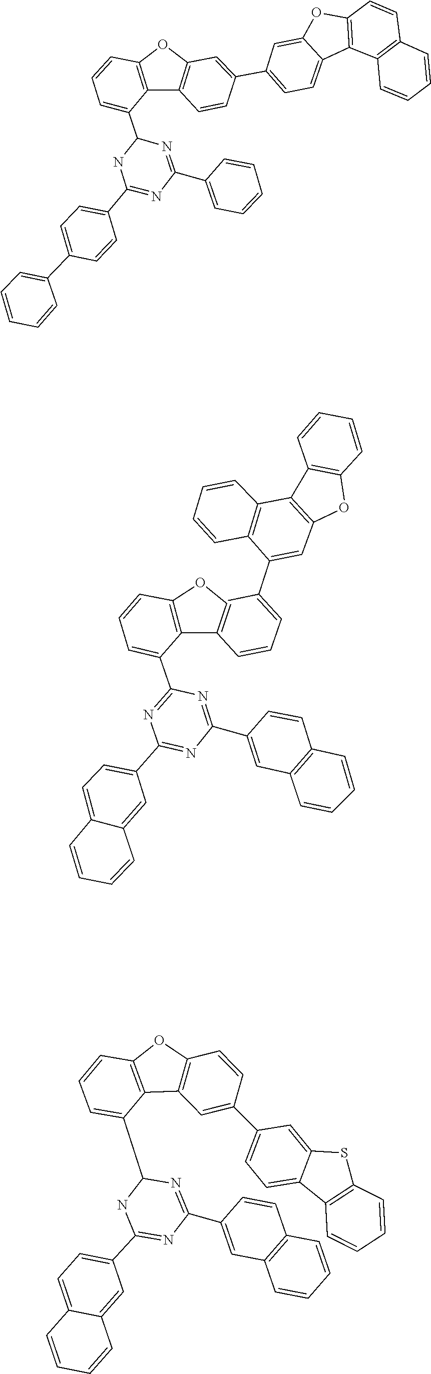

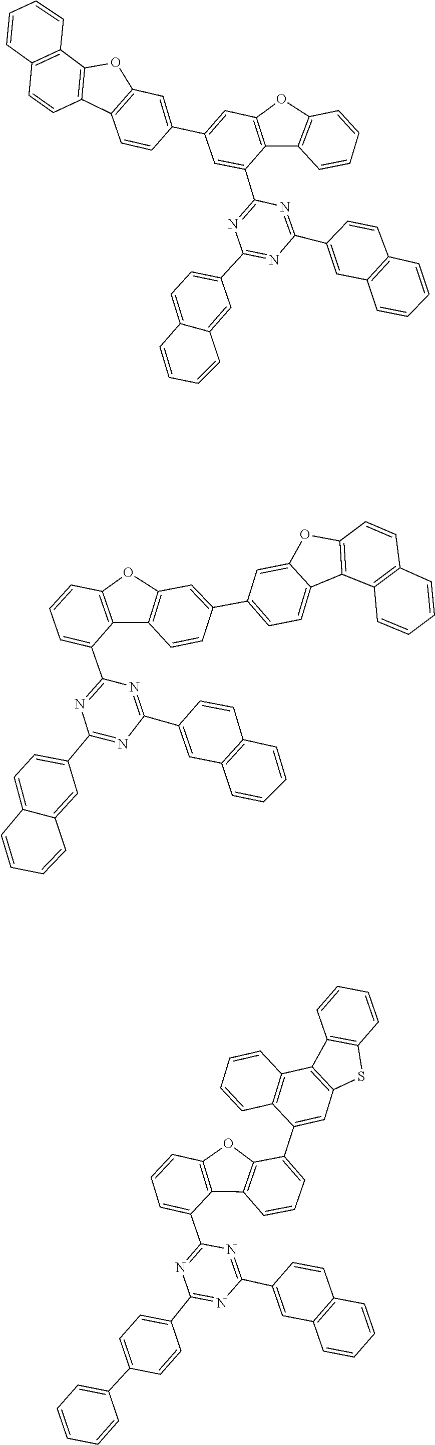 [SMILES: c1ccc2cc(-c3nc(-c4ccc5ccccc5c4)nc(-c4cc(-c5ccc6c(c5)oc5c7ccccc7ccc65)cc5oc6ccccc6c45)n3)ccc2c1 ; c1ccc2cc(-c3nc(-c4ccc5ccccc5c4)nc(-c4cccc5oc6cc(-c7ccc8c(c7)oc7ccc9ccccc9c78)ccc6c45)n3)ccc2c1 ; c1ccc(-c2ccc(-c3nc(-c4ccc5ccccc5c4)nc(-c4cccc5oc6c(-c7cc8sc9ccccc9c8c8ccccc78)cccc6c45)n3)cc2)cc1]

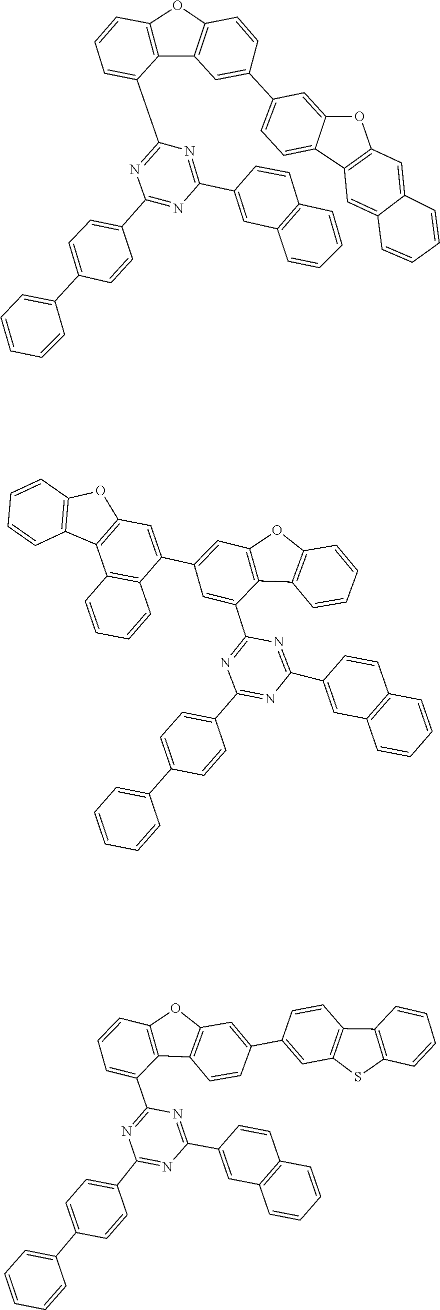 [SMILES: c1ccc(-c2ccc(-c3nc(-c4ccc5ccccc5c4)nc(-c4cccc5oc6ccc(-c7ccc8c(c7)oc7cc9ccccc9cc78)cc6c45)n3)cc2)cc1 ; c1ccc(-c2ccc(-c3nc(-c4ccc5ccccc5c4)nc(-c4cc(-c5cc6oc7ccccc7c6c6ccccc56)cc5oc6ccccc6c45)n3)cc2)cc1 ; c1ccc(-c2ccc(-c3nc(-c4ccc5ccccc5c4)nc(-c4cccc5oc6cc(-c7ccc8c(c7)sc7ccccc78)ccc6c45)n3)cc2)cc1]

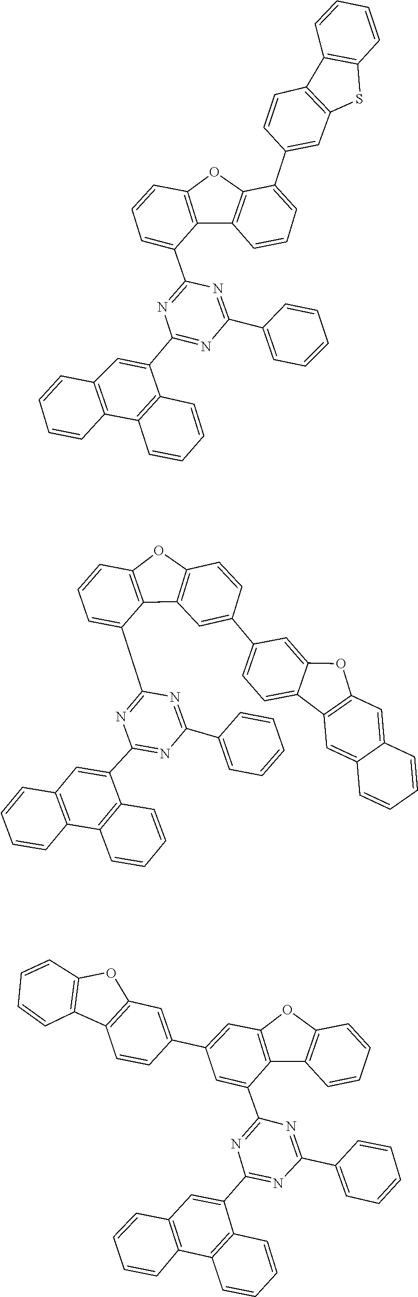 [SMILES: c1ccc(-c2nc(-c3cc4ccccc4c4ccccc34)nc(-c3cccc4oc5c(-c6ccc7c(c6)sc6ccccc67)cccc5c34)n2)cc1 ; c1ccc(-c2nc(-c3cc4ccccc4c4ccccc34)nc(-c3cccc4oc5ccc(-c6ccc7c(c6)oc6cc8ccccc8cc67)cc5c34)n2)cc1 ; c1ccc(-c2nc(-c3cc4ccccc4c4ccccc34)nc(-c3cc(-c4ccc5c(c4)oc4ccccc45)cc4oc5ccccc5c34)n2)cc1]

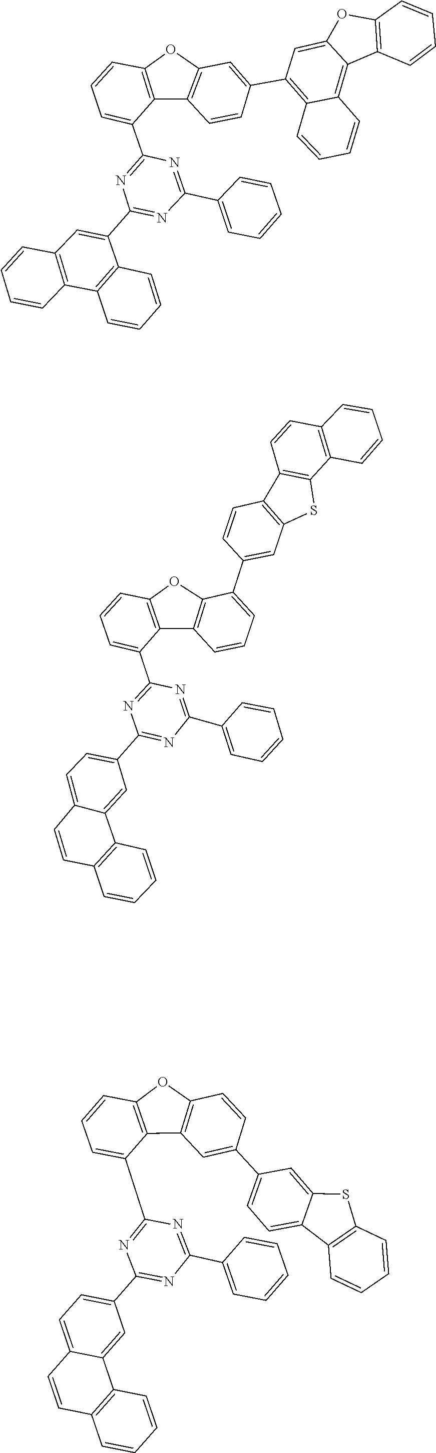 [SMILES: c1ccc(-c2nc(-c3cc4ccccc4c4ccccc34)nc(-c3cccc4oc5cc(-c6cc7oc8ccccc8c7c7ccccc67)ccc5c34)n2)cc1 ; c1ccc(-c2nc(-c3ccc4ccc5ccccc5c4c3)nc(-c3cccc4oc5c(-c6ccc7c(c6)sc6c8ccccc8ccc76)cccc5c34)n2)cc1 ; c1ccc(-c2nc(-c3ccc4ccc5ccccc5c4c3)nc(-c3cccc4oc5ccc(-c6ccc7c(c6)sc6ccccc67)cc5c34)n2)cc1]

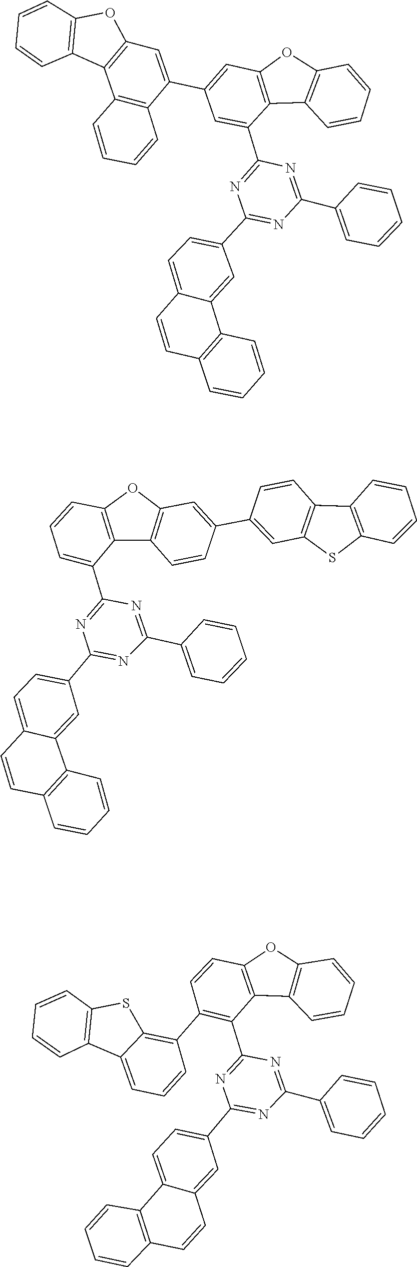 [SMILES: c1ccc(-c2nc(-c3ccc4ccc5ccccc5c4c3)nc(-c3cc(-c4cc5oc6ccccc6c5c5ccccc45)cc4oc5ccccc5c34)n2)cc1 ; c1ccc(-c2nc(-c3ccc4ccc5ccccc5c4c3)nc(-c3cccc4oc5cc(-c6ccc7c(c6)sc6ccccc67)ccc5c34)n2)cc1 ; c1ccc(-c2nc(-c3ccc4c(ccc5ccccc54)c3)nc(-c3c(-c4cccc5c4sc4ccccc45)ccc4oc5ccccc5c34)n2)cc1]

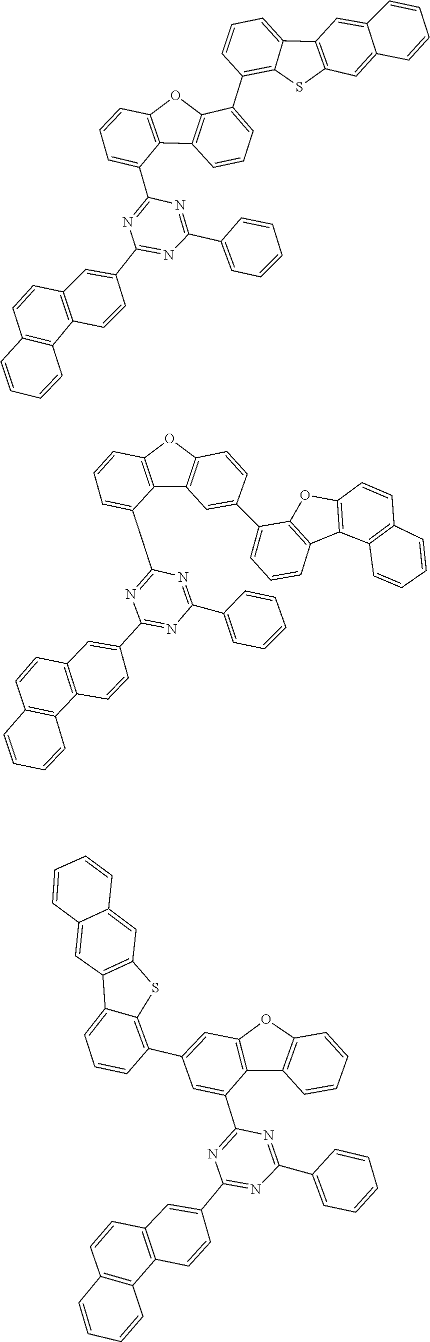 [SMILES: c1ccc(-c2nc(-c3ccc4c(ccc5ccccc54)c3)nc(-c3cccc4oc5c(-c6cccc7c6sc6cc8ccccc8cc67)cccc5c34)n2)cc1 ; c1ccc(-c2nc(-c3ccc4c(ccc5ccccc54)c3)nc(-c3cccc4oc5ccc(-c6cccc7c6oc6ccc8ccccc8c67)cc5c34)n2)cc1 ; c1ccc(-c2nc(-c3ccc4c(ccc5ccccc54)c3)nc(-c3cc(-c4cccc5c4sc4cc6ccccc6cc45)cc4oc5ccccc5c34)n2)cc1]

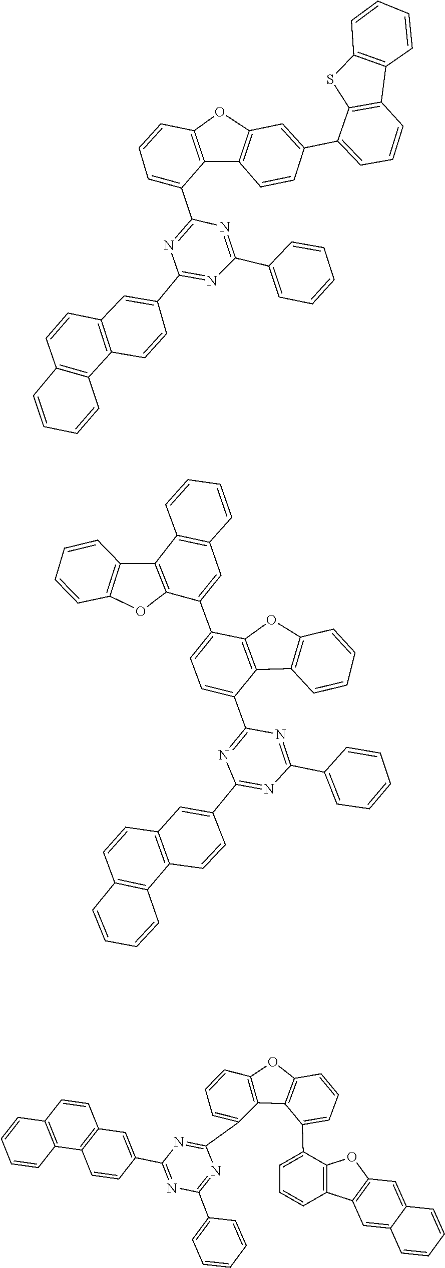 [SMILES: c1ccc(-c2nc(-c3ccc4c(ccc5ccccc54)c3)nc(-c3cccc4oc5cc(-c6cccc7c6sc6ccccc67)ccc5c34)n2)cc1 ; c1ccc(-c2nc(-c3ccc4c(ccc5ccccc54)c3)nc(-c3ccc(-c4cc5ccccc5c5c4oc4ccccc45)c4oc5ccccc5c34)n2)cc1 ; c1ccc(-c2nc(-c3ccc4c(ccc5ccccc54)c3)nc(-c3cccc4oc5cccc(-c6cccc7c6oc6cc8ccccc8cc67)c5c34)n2)cc1]

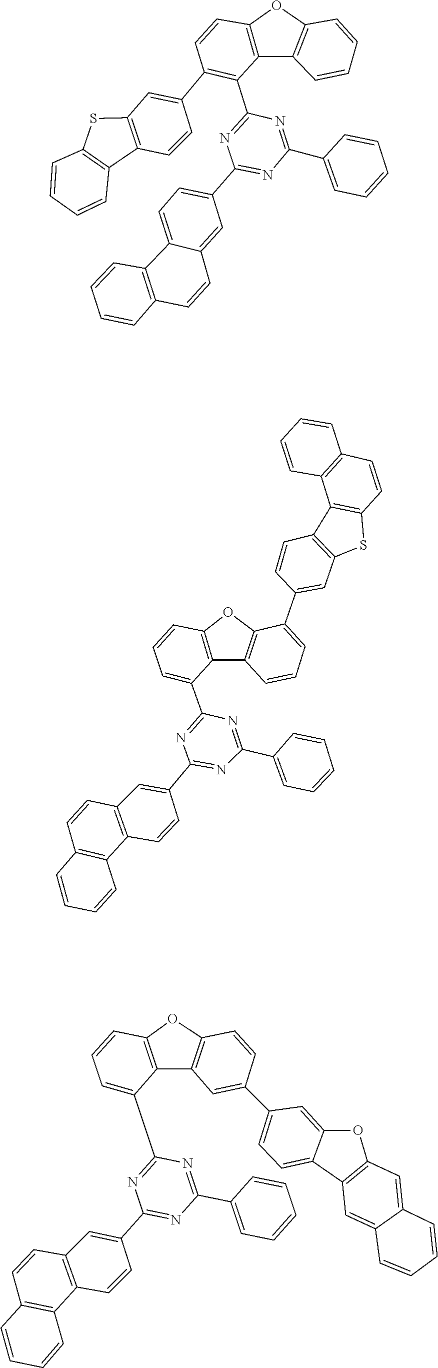 [SMILES: c1ccc(-c2nc(-c3ccc4c(ccc5ccccc54)c3)nc(-c3c(-c4ccc5c(c4)sc4ccccc45)ccc4oc5ccccc5c34)n2)cc1 ; c1ccc(-c2nc(-c3ccc4c(ccc5ccccc54)c3)nc(-c3cccc4oc5c(-c6ccc7c(c6)sc6ccc8ccccc8c67)cccc5c34)n2)cc1 ; c1ccc(-c2nc(-c3ccc4c(ccc5ccccc54)c3)nc(-c3cccc4oc5ccc(-c6ccc7c(c6)oc6cc8ccccc8cc67)cc5c34)n2)cc1]

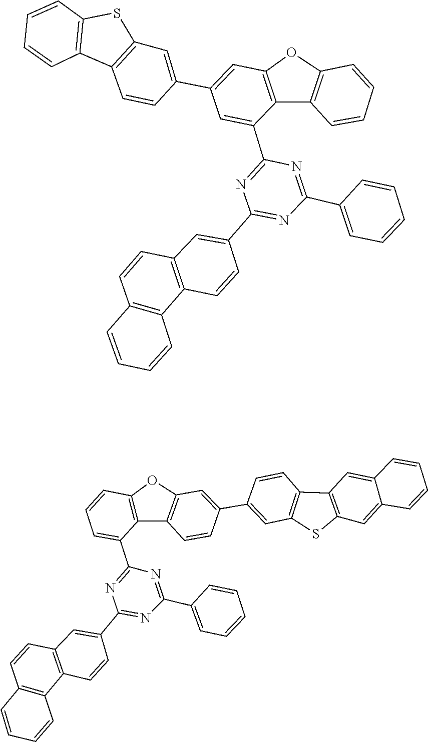 [SMILES: c1ccc(-c2nc(-c3ccc4c(ccc5ccccc54)c3)nc(-c3cc(-c4ccc5c(c4)sc4ccccc45)cc4oc5ccccc5c34)n2)cc1 ; c1ccc(-c2nc(-c3ccc4c(ccc5ccccc54)c3)nc(-c3cccc4oc5cc(-c6ccc7c(c6)sc6cc8ccccc8cc67)ccc5c34)n2)cc1]

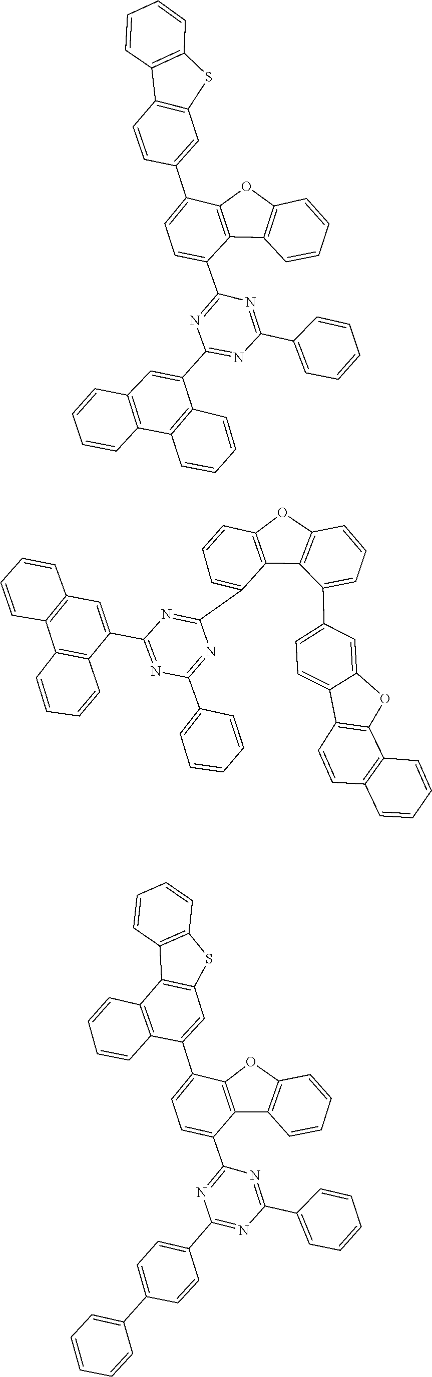 [SMILES: c1ccc(-c2nc(-c3cc4ccccc4c4ccccc34)nc(-c3ccc(-c4ccc5c(c4)sc4ccccc45)c4oc5ccccc5c34)n2)cc1 ; c1ccc(-c2nc(-c3cc4ccccc4c4ccccc34)nc(-c3cccc4oc5cccc(-c6ccc7c(c6)oc6c8ccccc8ccc76)c5c34)n2)cc1 ; c1ccc(-c2ccc(-c3nc(-c4ccccc4)nc(-c4ccc(-c5cc6sc7ccccc7c6c6ccccc56)c5oc6ccccc6c45)n3)cc2)cc1]

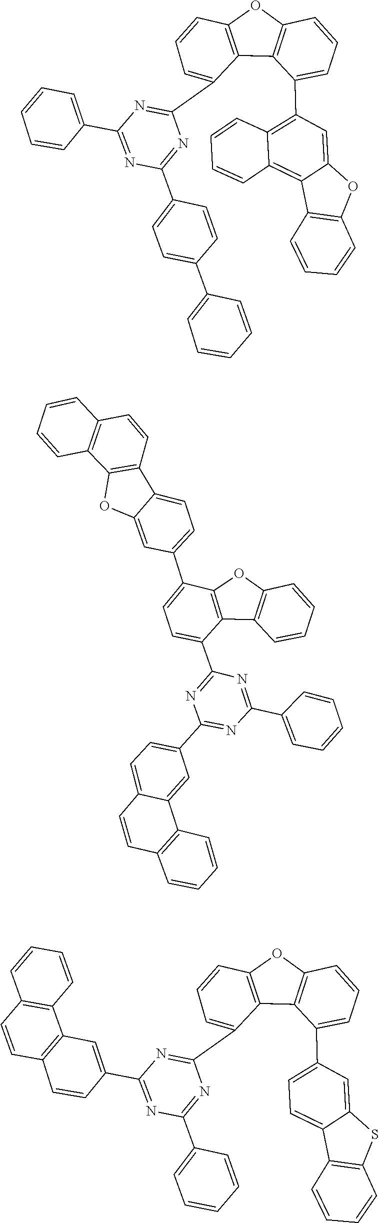 [SMILES: c1ccc(-c2ccc(-c3nc(-c4ccccc4)nc(-c4cccc5oc6cccc(-c7cc8oc9ccccc9c8c8ccccc78)c6c45)n3)cc2)cc1 ; c1ccc(-c2nc(-c3ccc4ccc5ccccc5c4c3)nc(-c3ccc(-c4ccc5c(c4)oc4c6ccccc6ccc54)c4oc5ccccc5c34)n2)cc1 ; c1ccc(-c2nc(-c3ccc4ccc5ccccc5c4c3)nc(-c3cccc4oc5cccc(-c6ccc7c(c6)sc6ccccc67)c5c34)n2)cc1]

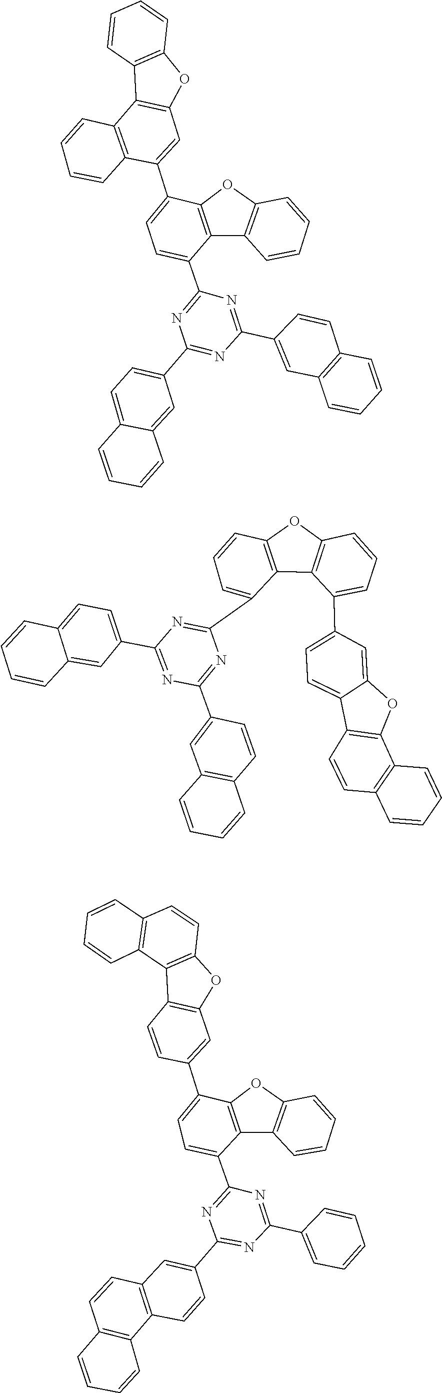 [SMILES: c1ccc2cc(-c3nc(-c4ccc5ccccc5c4)nc(-c4ccc(-c5cc6oc7ccccc7c6c6ccccc56)c5oc6ccccc6c45)n3)ccc2c1 ; c1ccc2cc(-c3nc(-c4ccc5ccccc5c4)nc(-c4cccc5oc6cccc(-c7ccc8c(c7)oc7c9ccccc9ccc87)c6c45)n3)ccc2c1 ; c1ccc(-c2nc(-c3ccc4c(ccc5ccccc54)c3)nc(-c3ccc(-c4ccc5c(c4)oc4ccc6ccccc6c45)c4oc5ccccc5c34)n2)cc1]

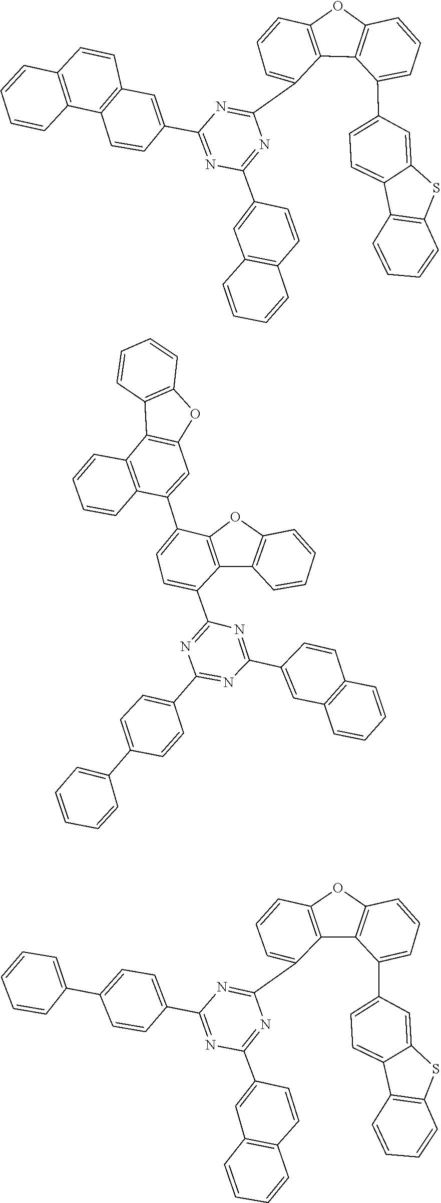 [SMILES: c1ccc2cc(-c3nc(-c4ccc5c(ccc6ccccc65)c4)nc(-c4cccc5oc6cccc(-c7ccc8c(c7)sc7ccccc78)c6c45)n3)ccc2c1 ; c1ccc(-c2ccc(-c3nc(-c4ccc5ccccc5c4)nc(-c4ccc(-c5cc6oc7ccccc7c6c6ccccc56)c5oc6ccccc6c45)n3)cc2)cc1 ; c1ccc(-c2ccc(-c3nc(-c4ccc5ccccc5c4)nc(-c4cccc5oc6cccc(-c7ccc8c(c7)sc7ccccc78)c6c45)n3)cc2)cc1]

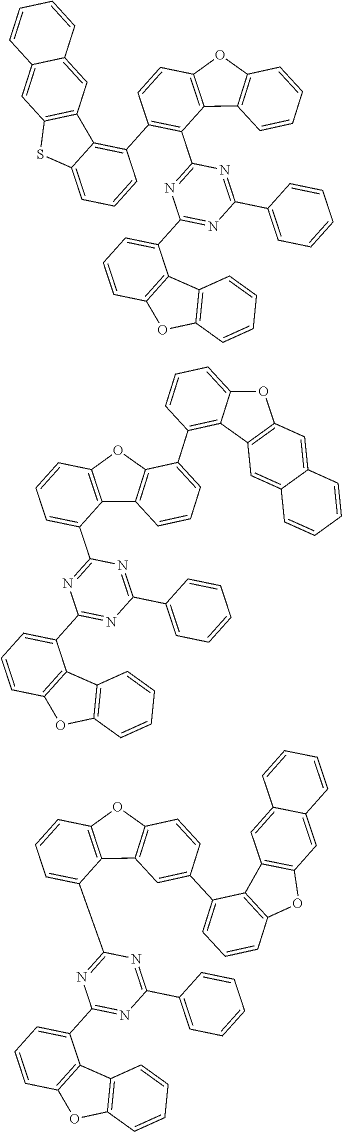

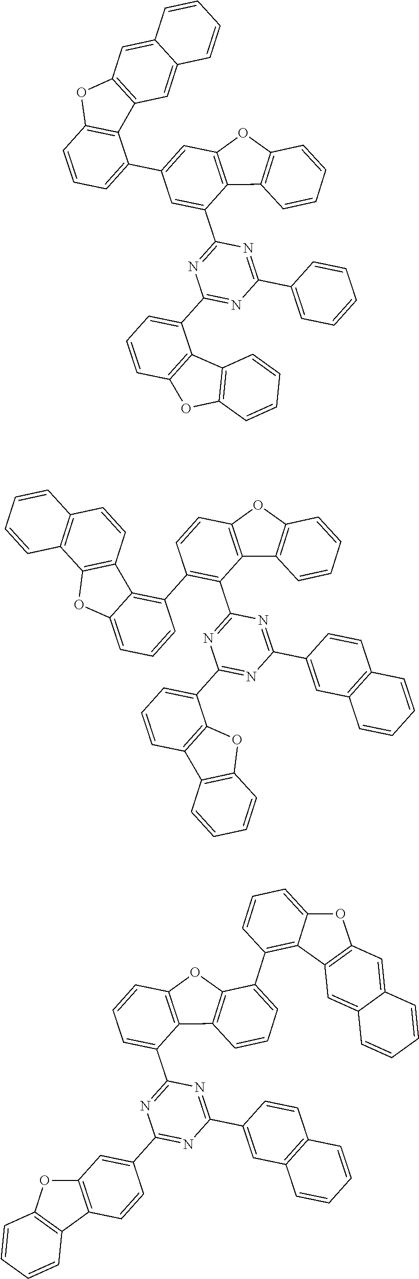 [SMILES: c1ccc(-c2nc(-c3cccc4oc5ccccc5c34)nc(-c3cc(-c4cccc5oc6cc7ccccc7cc6c45)cc4oc5ccccc5c34)n2)cc1 ; c1ccc2cc(-c3nc(-c4cccc5c4oc4ccccc45)nc(-c4c(-c5cccc6oc7c8ccccc8ccc7c56)ccc5oc6ccccc6c45)n3)ccc2c1 ; c1ccc2cc(-c3nc(-c4ccc5c(c4)oc4ccccc45)nc(-c4cccc5oc6c(-c7cccc8oc9cc%10ccccc%10cc9c78)cccc6c45)n3)ccc2c1]

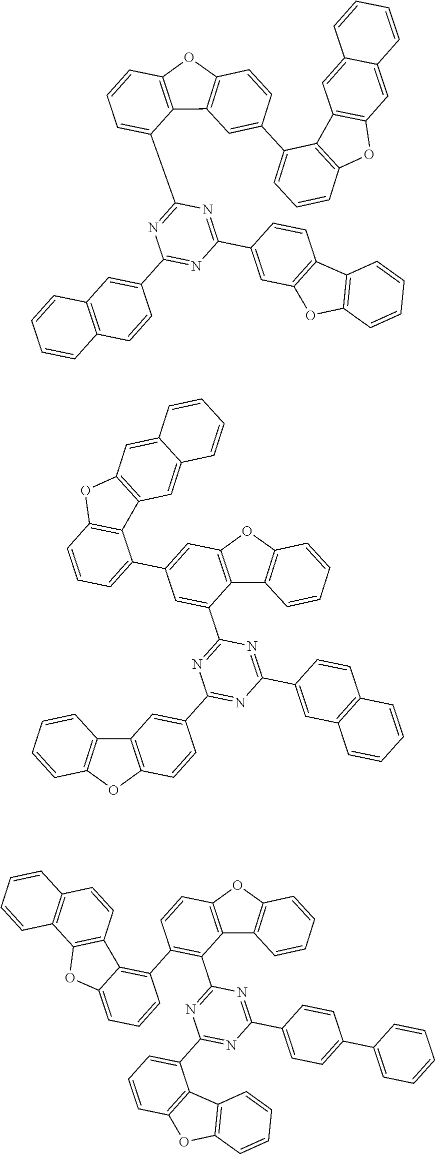 [SMILES: c1ccc2cc(-c3nc(-c4ccc5c(c4)oc4ccccc45)nc(-c4cccc5oc6ccc(-c7cccc8oc9cc%10ccccc%10cc9c78)cc6c45)n3)ccc2c1 ; c1ccc2cc(-c3nc(-c4ccc5oc6ccccc6c5c4)nc(-c4cc(-c5cccc6oc7cc8ccccc8cc7c56)cc5oc6ccccc6c45)n3)ccc2c1 ; c1ccc(-c2ccc(-c3nc(-c4cccc5oc6ccccc6c45)nc(-c4c(-c5cccc6oc7c8ccccc8ccc7c56)ccc5oc6ccccc6c45)n3)cc2)cc1]

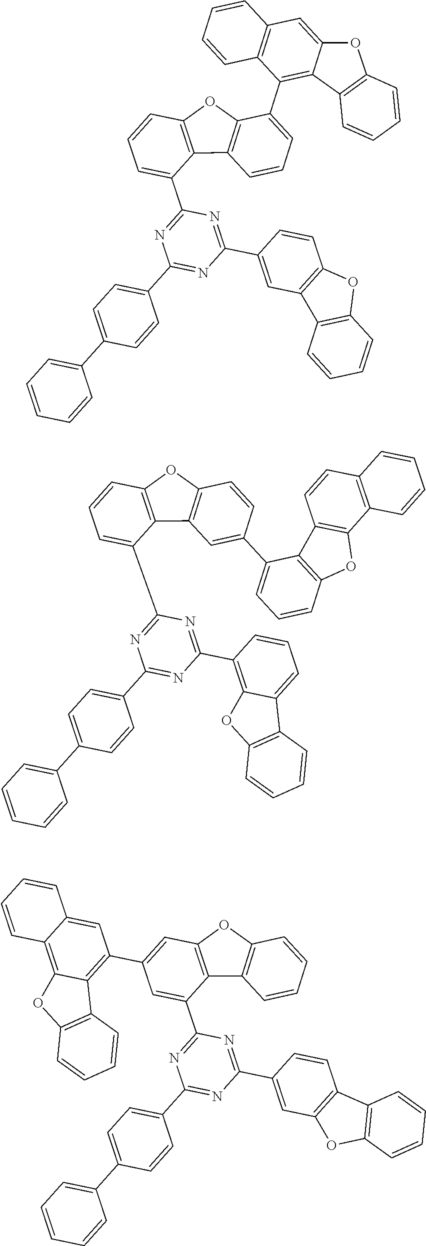 [SMILES: c1ccc(-c2ccc(-c3nc(-c4ccc5oc6ccccc6c5c4)nc(-c4cccc5oc6c(-c7c8ccccc8cc8oc9ccccc9c78)cccc6c45)n3)cc2)cc1 ; c1ccc(-c2ccc(-c3nc(-c4cccc5c4oc4ccccc45)nc(-c4cccc5oc6ccc(-c7cccc8oc9c%10ccccc%10ccc9c78)cc6c45)n3)cc2)cc1 ; c1ccc(-c2ccc(-c3nc(-c4ccc5c(c4)oc4ccccc45)nc(-c4cc(-c5cc6ccccc6c6oc7ccccc7c56)cc5oc6ccccc6c45)n3)cc2)cc1]

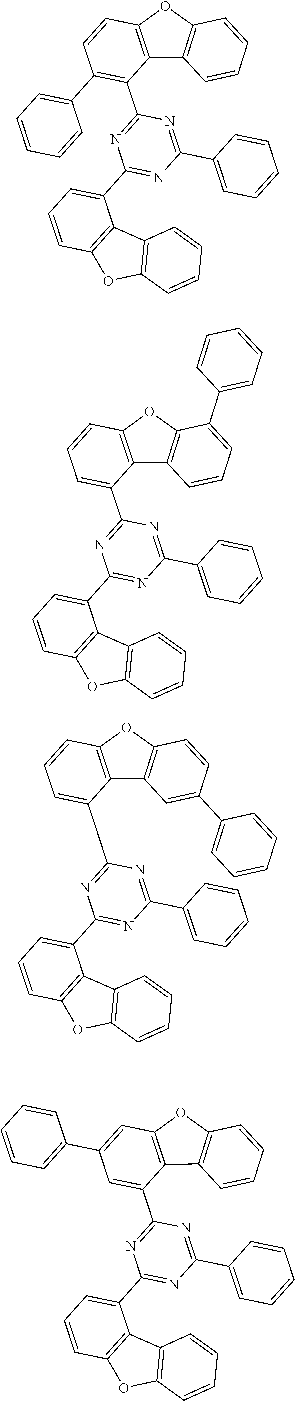

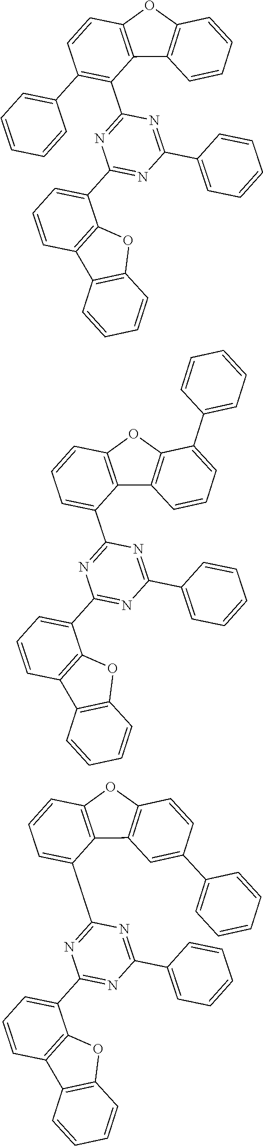

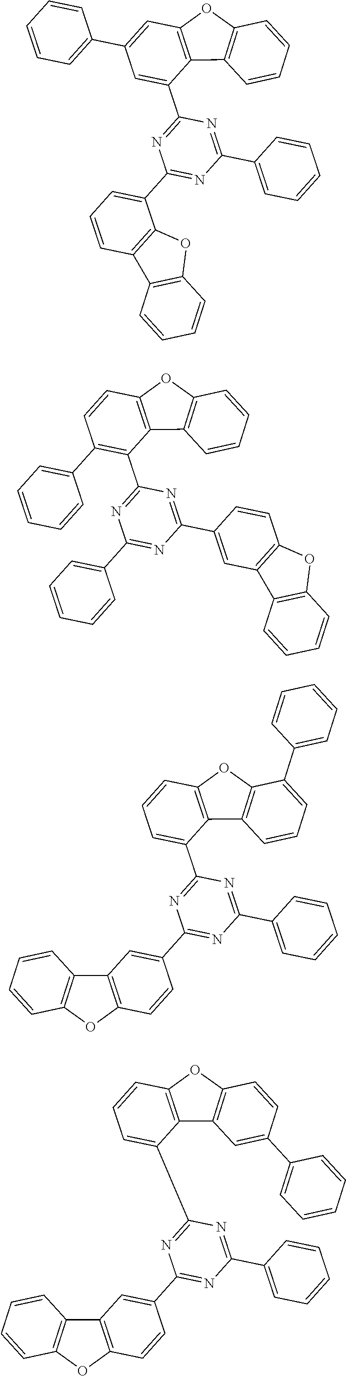

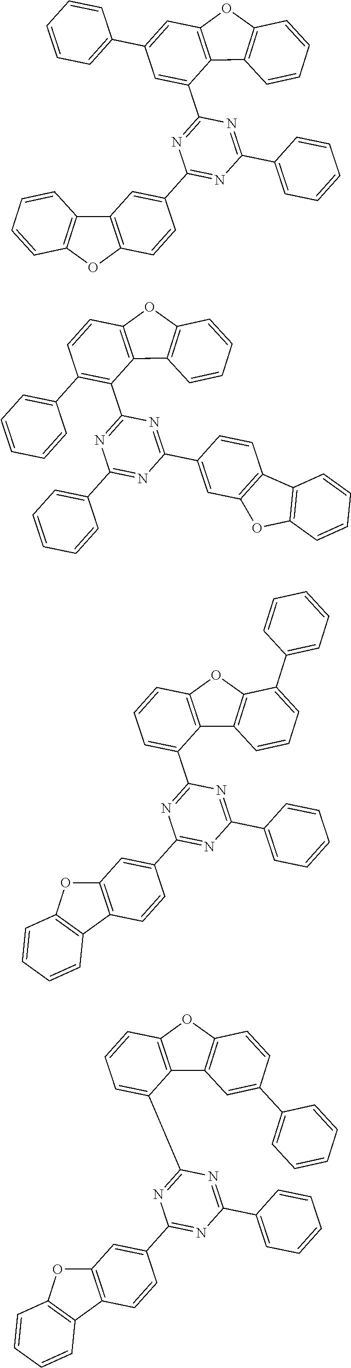 [SMILES: c1ccc(-c2cc(-c3nc(-c4ccccc4)nc(-c4ccc5oc6ccccc6c5c4)n3)c3c(c2)oc2ccccc23)cc1 ; c1ccc(-c2nc(-c3ccc4c(c3)oc3ccccc34)nc(-c3c(-c4ccccc4)ccc4oc5ccccc5c34)n2)cc1 ; c1ccc(-c2nc(-c3ccc4c(c3)oc3ccccc34)nc(-c3cccc4oc5c(-c6ccccc6)cccc5c34)n2)cc1 ; c1ccc(-c2ccc3oc4cccc(-c5nc(-c6ccccc6)nc(-c6ccc7c(c6)oc6ccccc67)n5)c4c3c2)cc1]

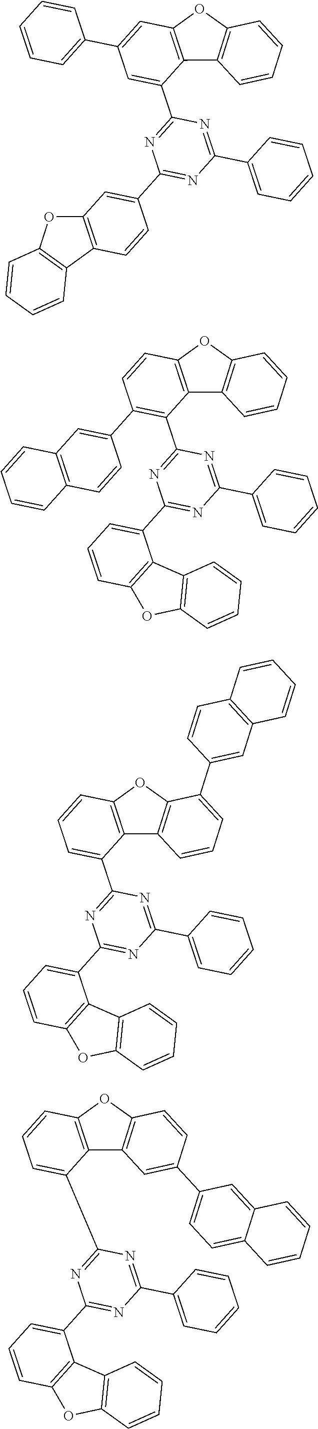

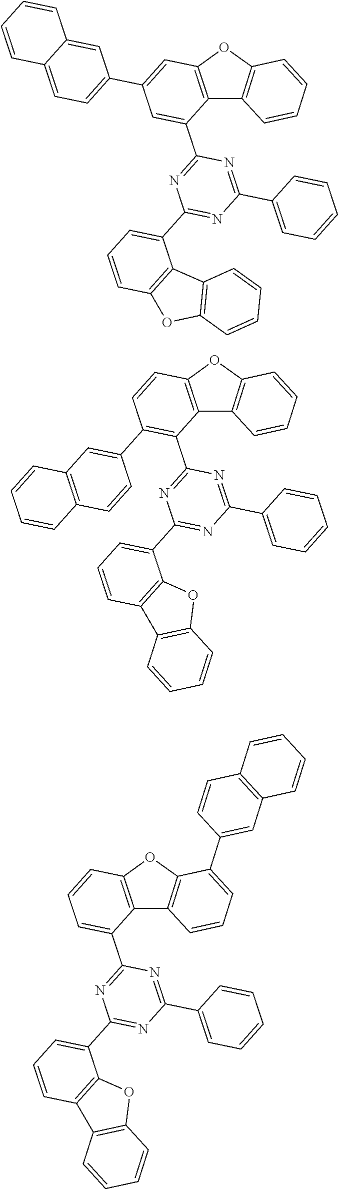

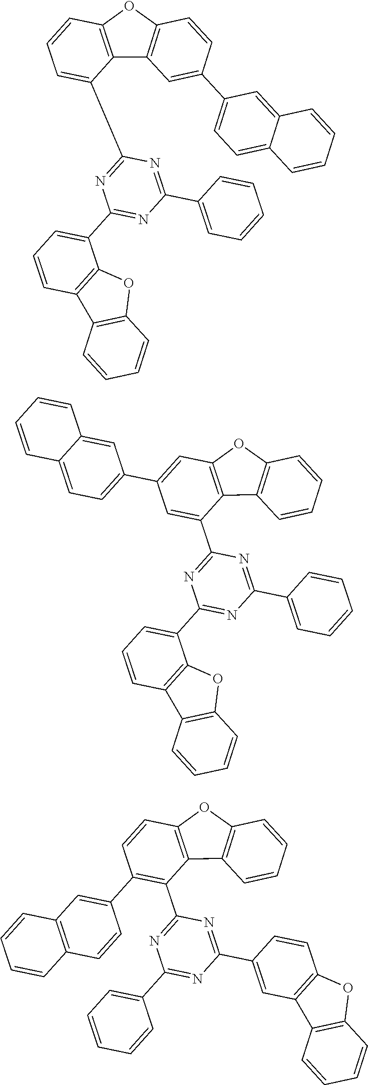

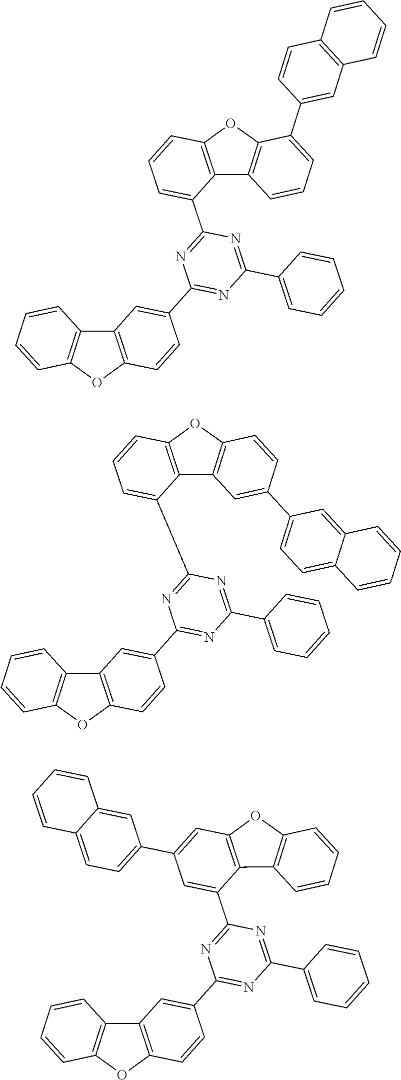 [SMILES: c1ccc(-c2nc(-c3ccc4oc5ccccc5c4c3)nc(-c3cccc4oc5c(-c6ccc7ccccc7c6)cccc5c34)n2)cc1 ; c1ccc(-c2nc(-c3ccc4oc5ccccc5c4c3)nc(-c3cccc4oc5ccc(-c6ccc7ccccc7c6)cc5c34)n2)cc1 ; c1ccc(-c2nc(-c3ccc4oc5ccccc5c4c3)nc(-c3cc(-c4ccc5ccccc5c4)cc4oc5ccccc5c34)n2)cc1]

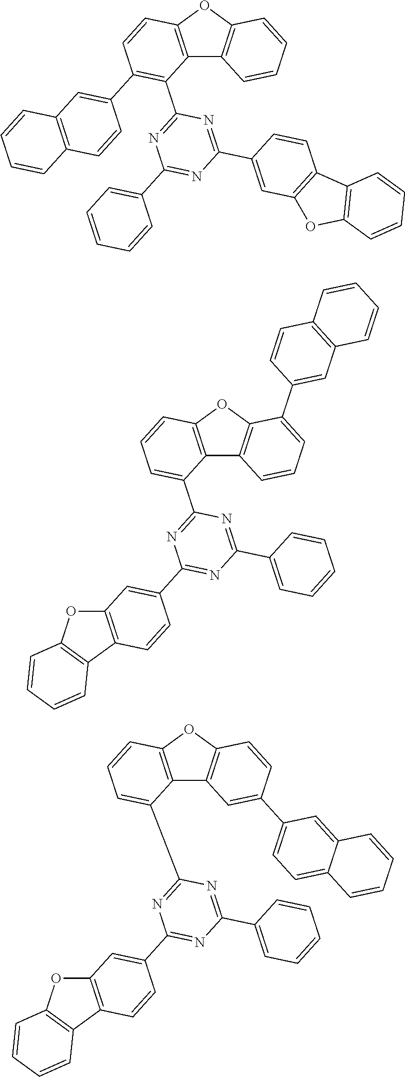

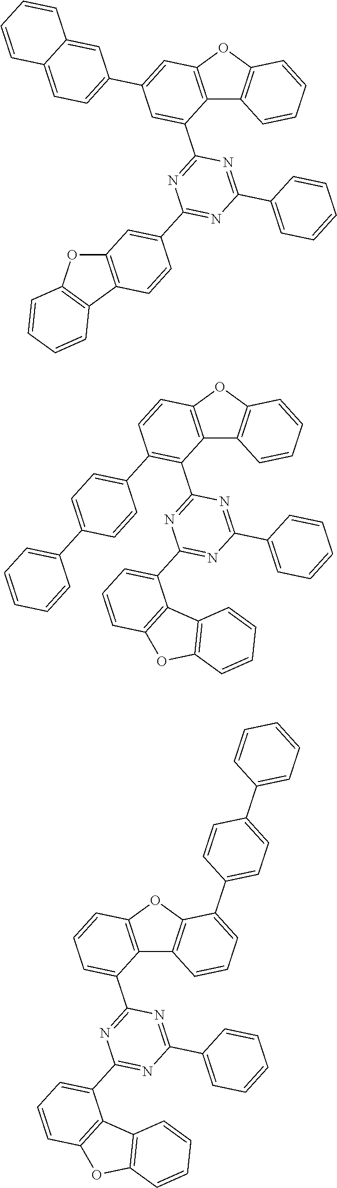 [SMILES: c1ccc(-c2nc(-c3ccc4c(c3)oc3ccccc34)nc(-c3cc(-c4ccc5ccccc5c4)cc4oc5ccccc5c34)n2)cc1 ; c1ccc(-c2ccc(-c3ccc4oc5ccccc5c4c3-c3nc(-c4ccccc4)nc(-c4cccc5oc6ccccc6c45)n3)cc2)cc1 ; c1ccc(-c2ccc(-c3cccc4c3oc3cccc(-c5nc(-c6ccccc6)nc(-c6cccc7oc8ccccc8c67)n5)c34)cc2)cc1]

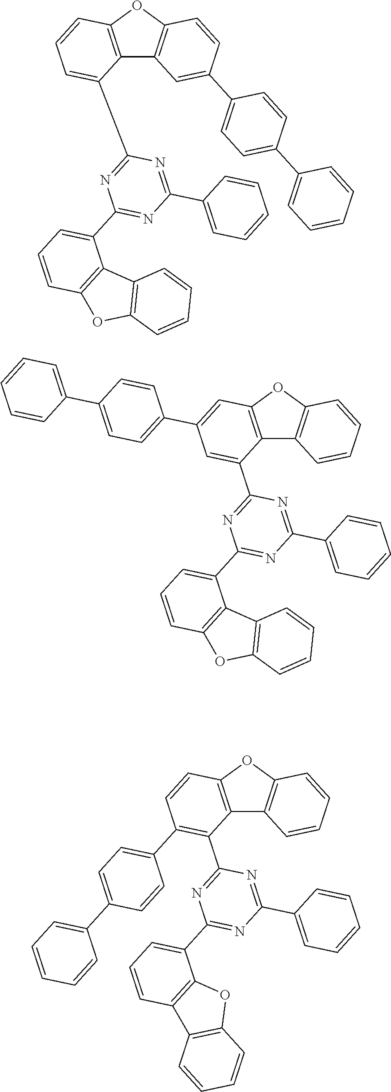 [SMILES: c1ccc(-c2ccc(-c3ccc4oc5cccc(-c6nc(-c7ccccc7)nc(-c7cccc8oc9ccccc9c78)n6)c5c4c3)cc2)cc1 ; c1ccc(-c2ccc(-c3cc(-c4nc(-c5ccccc5)nc(-c5cccc6oc7ccccc7c56)n4)c4c(c3)oc3ccccc34)cc2)cc1 ; c1ccc(-c2ccc(-c3ccc4oc5ccccc5c4c3-c3nc(-c4ccccc4)nc(-c4cccc5c4oc4ccccc45)n3)cc2)cc1]

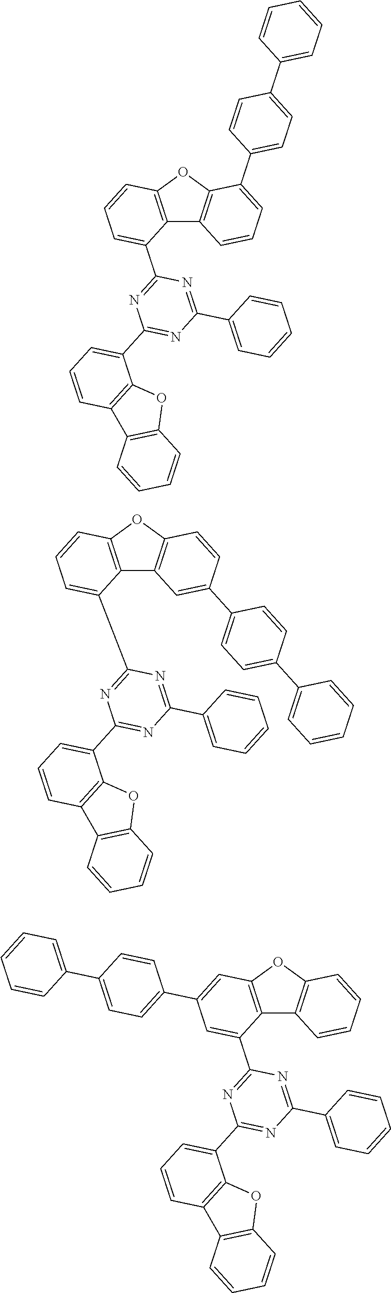

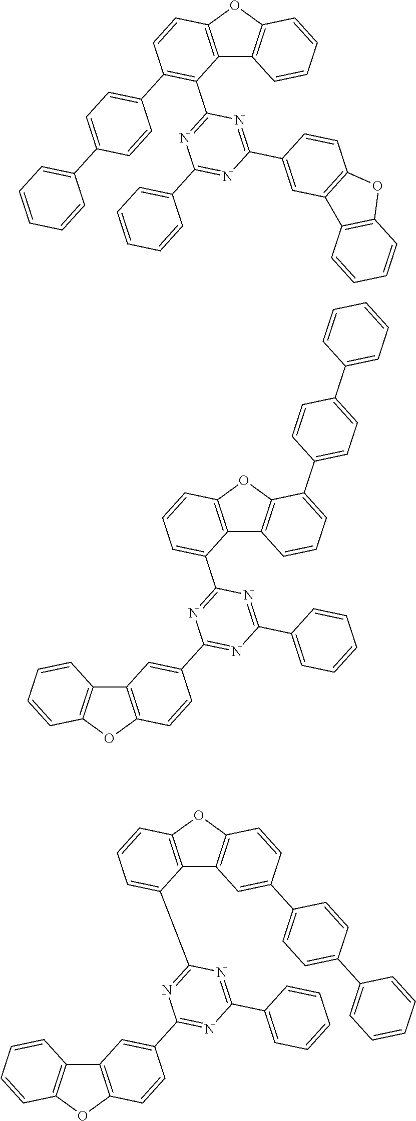

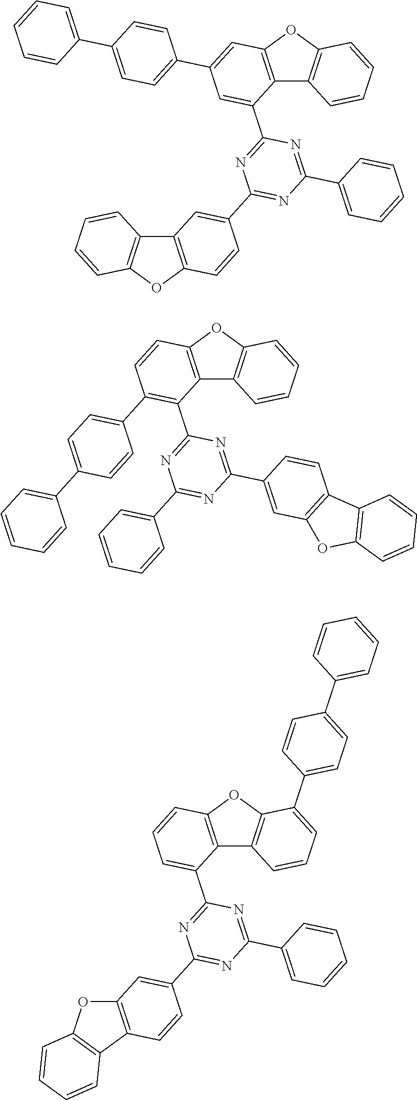 [SMILES: c1ccc(-c2ccc(-c3cc(-c4nc(-c5ccccc5)nc(-c5ccc6oc7ccccc7c6c5)n4)c4c(c3)oc3ccccc34)cc2)cc1 ; c1ccc(-c2ccc(-c3ccc4oc5ccccc5c4c3-c3nc(-c4ccccc4)nc(-c4ccc5c(c4)oc4ccccc45)n3)cc2)cc1 ; c1ccc(-c2ccc(-c3cccc4c3oc3cccc(-c5nc(-c6ccccc6)nc(-c6ccc7c(c6)oc6ccccc67)n5)c34)cc2)cc1]

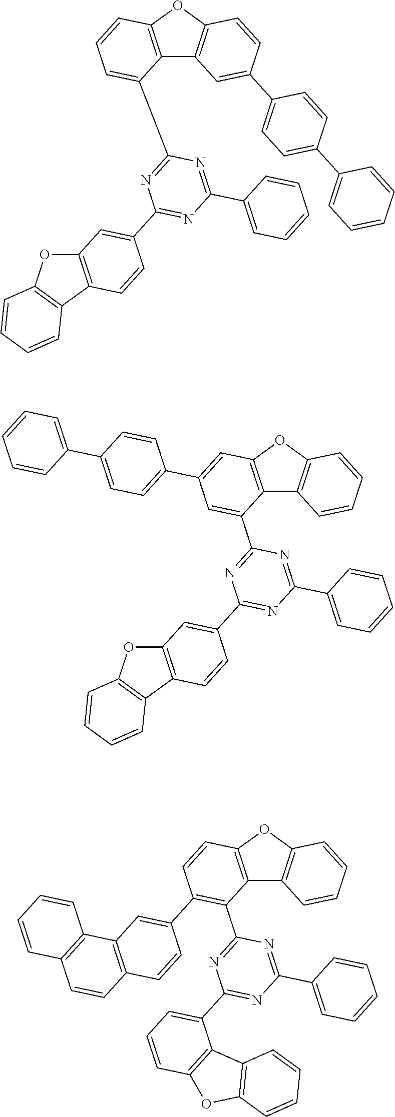 [SMILES: c1ccc(-c2ccc(-c3ccc4oc5cccc(-c6nc(-c7ccccc7)nc(-c7ccc8c(c7)oc7ccccc78)n6)c5c4c3)cc2)cc1 ; c1ccc(-c2ccc(-c3cc(-c4nc(-c5ccccc5)nc(-c5ccc6c(c5)oc5ccccc56)n4)c4c(c3)oc3ccccc34)cc2)cc1 ; c1ccc(-c2nc(-c3cccc4oc5ccccc5c34)nc(-c3c(-c4ccc5ccc6ccccc6c5c4)ccc4oc5ccccc5c34)n2)cc1]

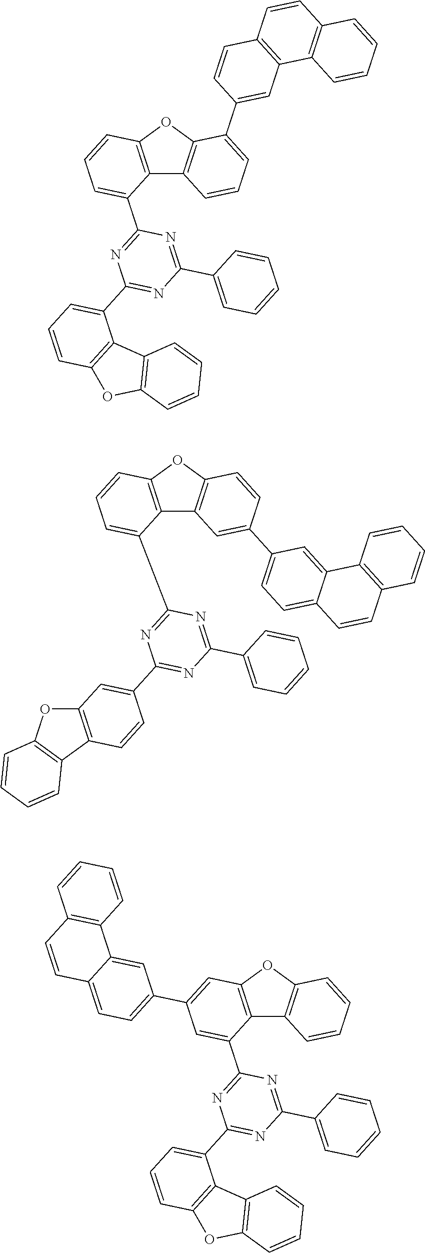 [SMILES: c1ccc(-c2nc(-c3cccc4oc5ccccc5c34)nc(-c3cccc4oc5c(-c6ccc7ccc8ccccc8c7c6)cccc5c34)n2)cc1 ; c1ccc(-c2nc(-c3ccc4c(c3)oc3ccccc34)nc(-c3cccc4oc5ccc(-c6ccc7ccc8ccccc8c7c6)cc5c34)n2)cc1 ; c1ccc(-c2nc(-c3cccc4oc5ccccc5c34)nc(-c3cc(-c4ccc5ccc6ccccc6c5c4)cc4oc5ccccc5c34)n2)cc1]

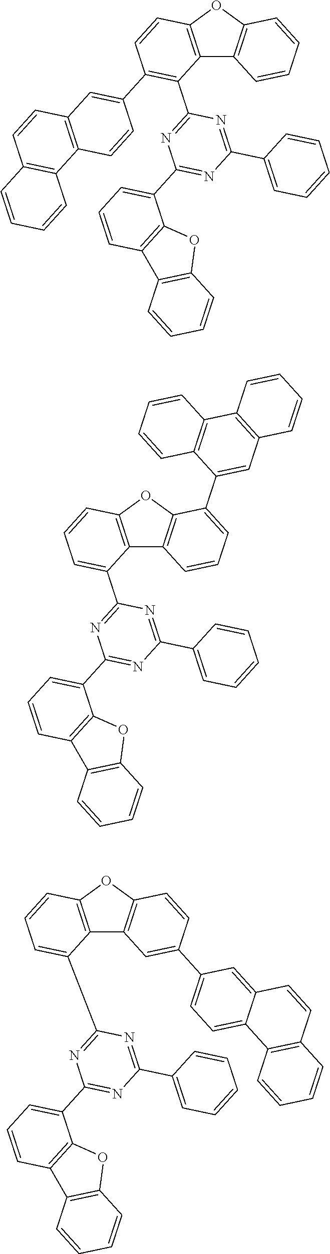 [SMILES: c1ccc(-c2nc(-c3cccc4c3oc3ccccc34)nc(-c3c(-c4ccc5c(ccc6ccccc65)c4)ccc4oc5ccccc5c34)n2)cc1 ; c1ccc(-c2nc(-c3cccc4c3oc3ccccc34)nc(-c3cccc4oc5c(-c6cc7ccccc7c7ccccc67)cccc5c34)n2)cc1 ; c1ccc(-c2nc(-c3cccc4c3oc3ccccc34)nc(-c3cccc4oc5ccc(-c6ccc7c(ccc8ccccc87)c6)cc5c34)n2)cc1]

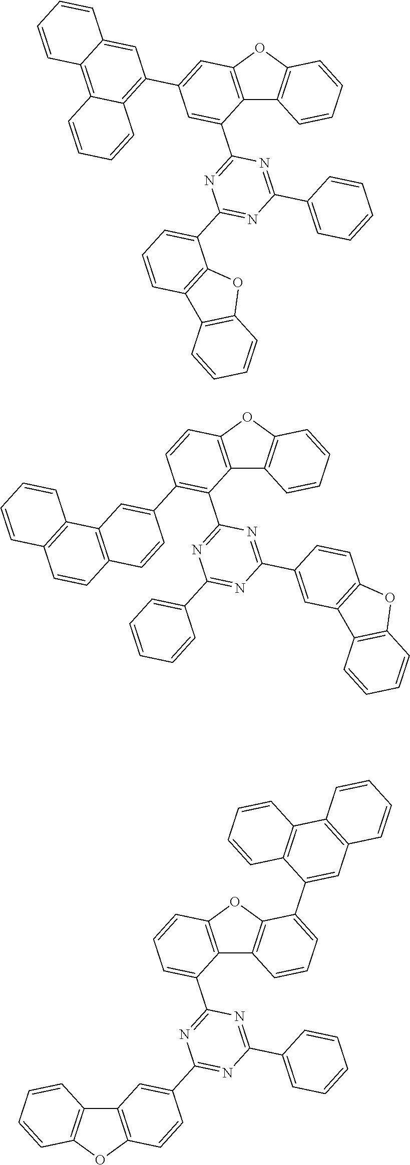

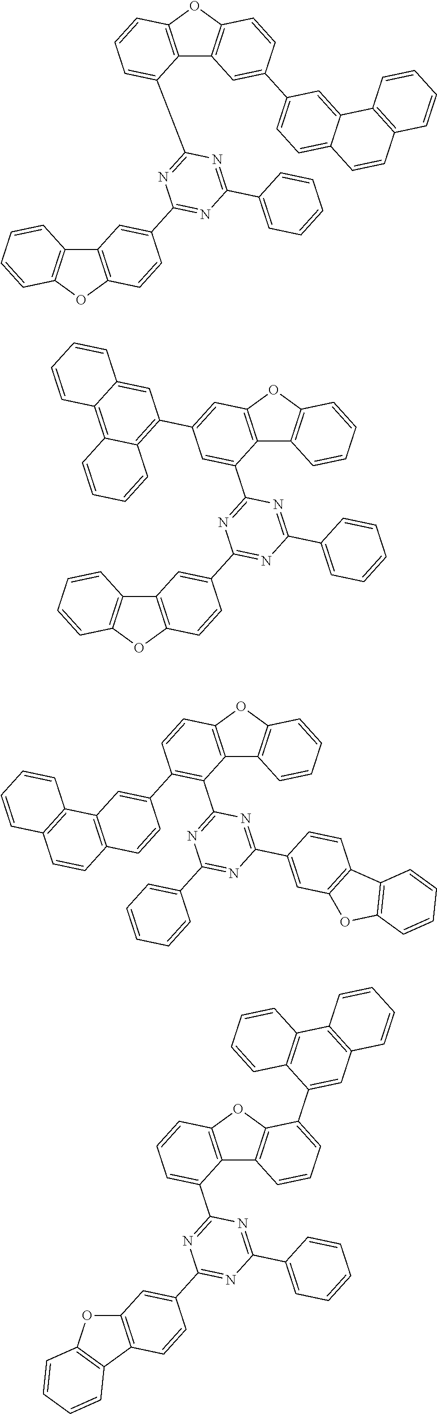

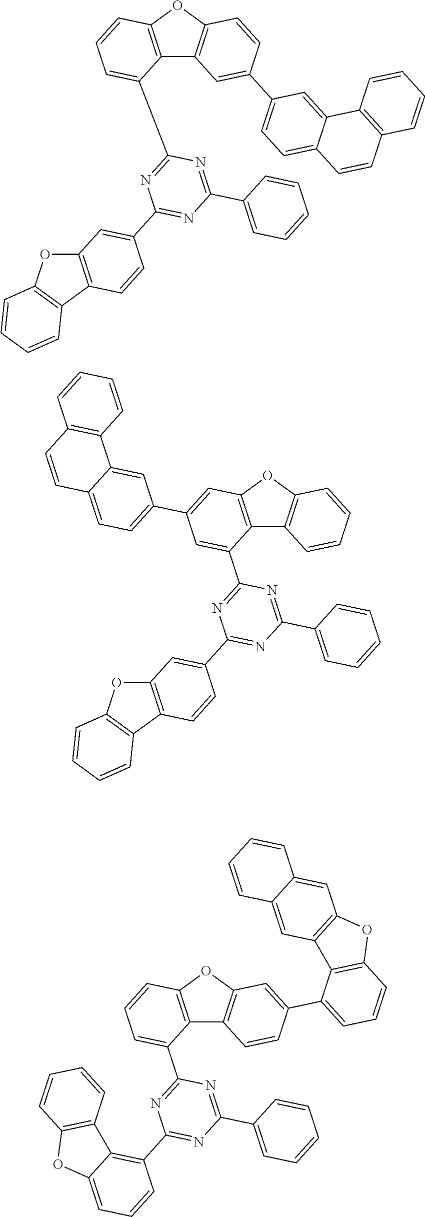

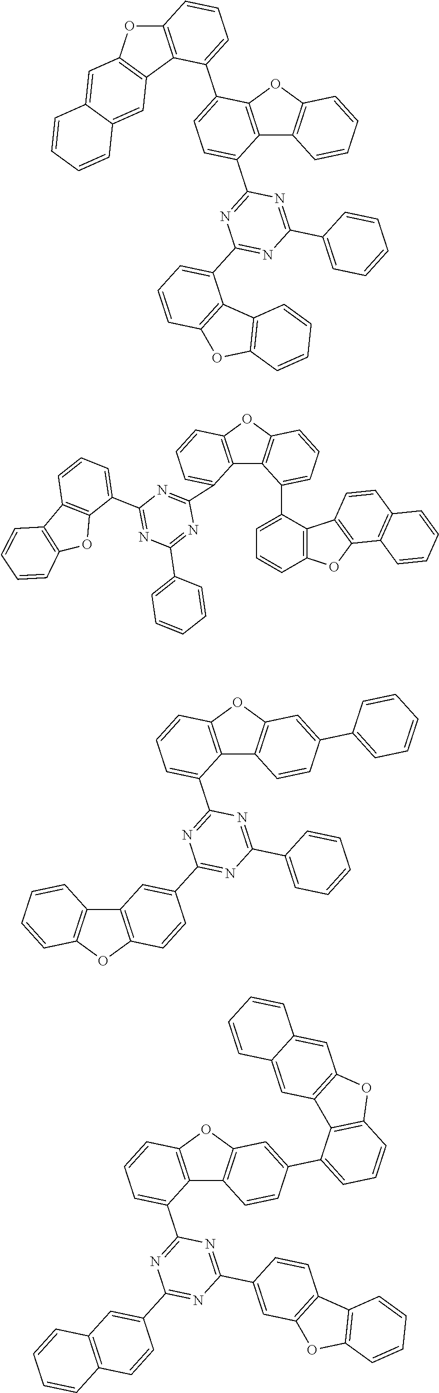 [SMILES: c1ccc(-c2nc(-c3cccc4oc5ccccc5c34)nc(-c3ccc(-c4cccc5oc6cc7ccccc7cc6c45)c4oc5ccccc5c34)n2)cc1 ; c1ccc(-c2nc(-c3cccc4c3oc3ccccc34)nc(-c3cccc4oc5cccc(-c6cccc7oc8c9ccccc9ccc8c67)c5c34)n2)cc1 ; c1ccc(-c2ccc3c(c2)oc2cccc(-c4nc(-c5ccccc5)nc(-c5ccc6oc7ccccc7c6c5)n4)c23)cc1 ; c1ccc2cc(-c3nc(-c4ccc5c(c4)oc4ccccc45)nc(-c4cccc5oc6cc(-c7cccc8oc9cc%10ccccc%10cc9c78)ccc6c45)n3)ccc2c1]

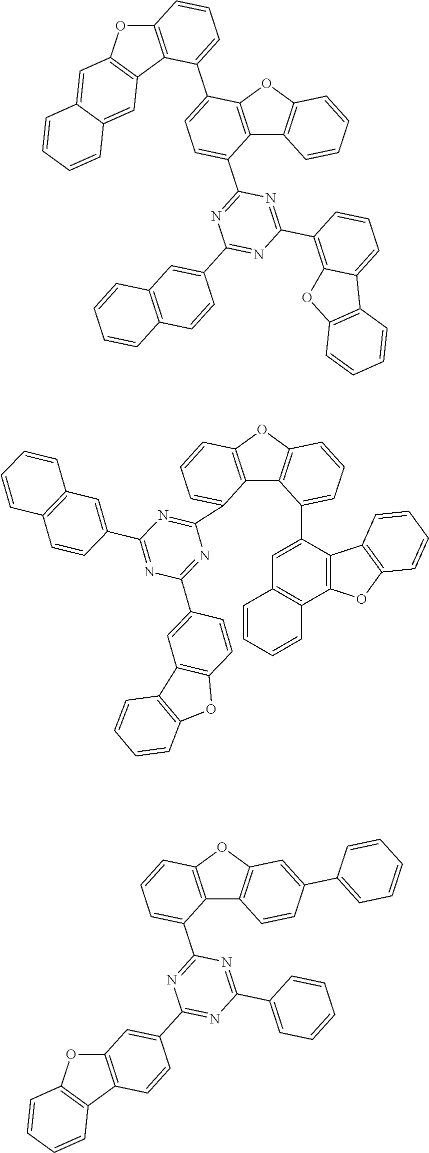 [SMILES: c1ccc2cc(-c3nc(-c4cccc5c4oc4ccccc45)nc(-c4ccc(-c5cccc6oc7cc8ccccc8cc7c56)c5oc6ccccc6c45)n3)ccc2c1 ; c1ccc2cc(-c3nc(-c4ccc5oc6ccccc6c5c4)nc(-c4cccc5oc6cccc(-c7cc8ccccc8c8oc9ccccc9c78)c6c45)n3)ccc2c1 ; c1ccc(-c2ccc3c(c2)oc2cccc(-c4nc(-c5ccccc5)nc(-c5ccc6c(c5)oc5ccccc56)n4)c23)cc1]

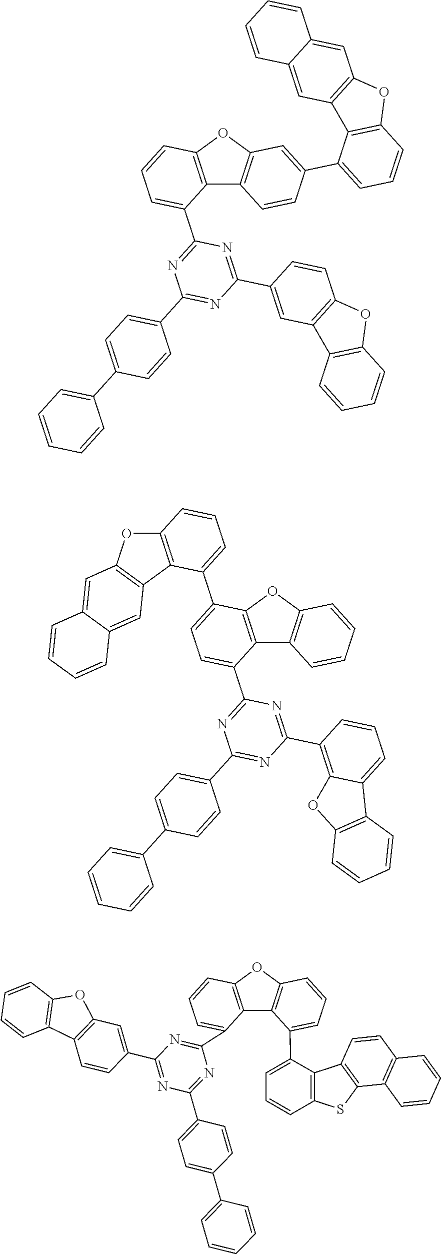 [SMILES: c1ccc(-c2ccc(-c3nc(-c4ccc5oc6ccccc6c5c4)nc(-c4cccc5oc6cc(-c7cccc8oc9cc%10ccccc%10cc9c78)ccc6c45)n3)cc2)cc1 ; c1ccc(-c2ccc(-c3nc(-c4cccc5c4oc4ccccc45)nc(-c4ccc(-c5cccc6oc7cc8ccccc8cc7c56)c5oc6ccccc6c45)n3)cc2)cc1 ; c1ccc(-c2ccc(-c3nc(-c4ccc5c(c4)oc4ccccc45)nc(-c4cccc5oc6cccc(-c7cccc8sc9c%10ccccc%10ccc9c78)c6c45)n3)cc2)cc1]

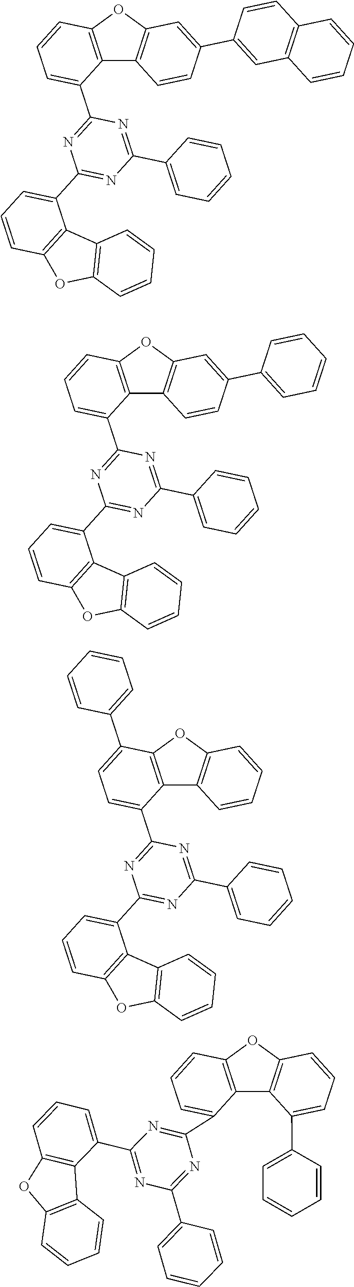

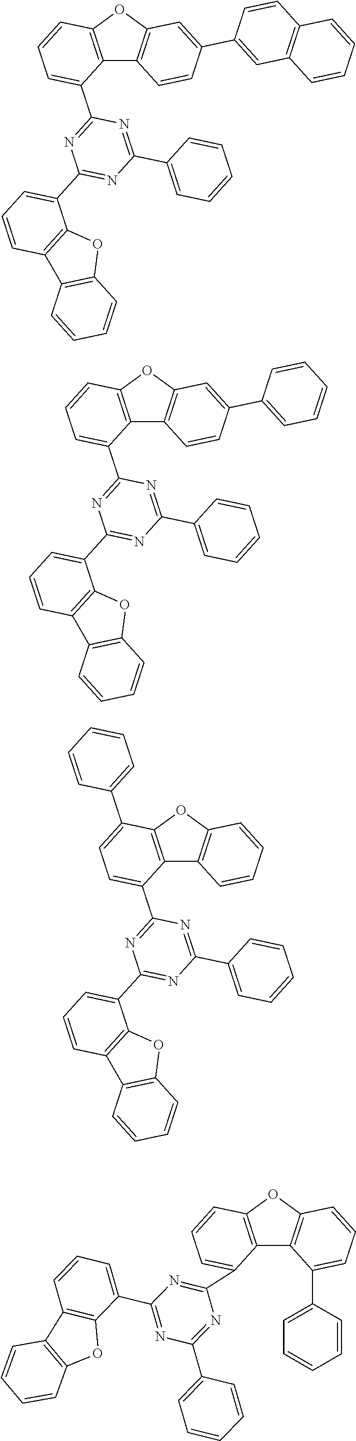

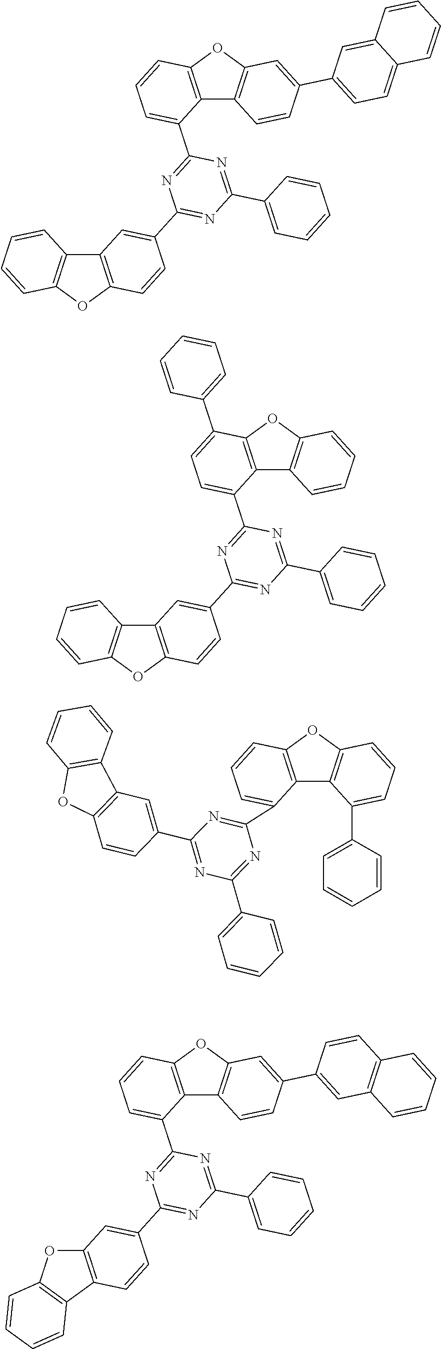

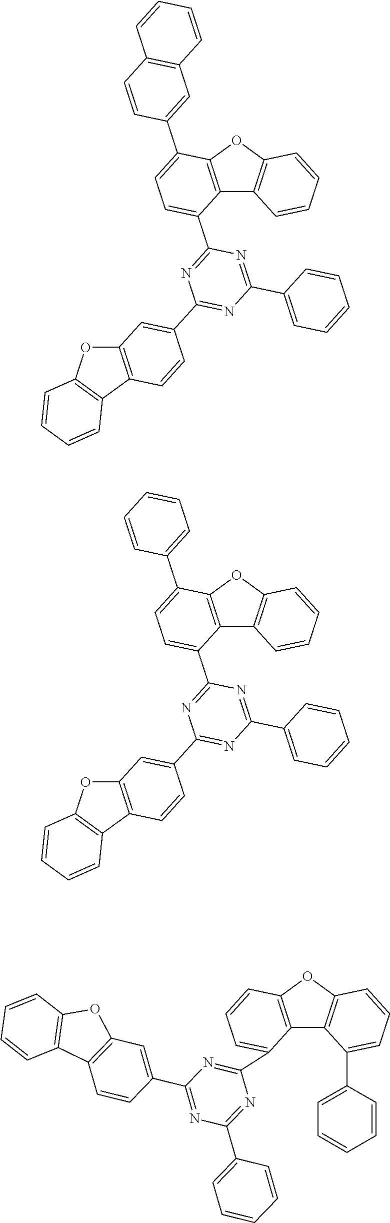 [SMILES: c1ccc(-c2nc(-c3ccc4c(c3)oc3ccccc34)nc(-c3ccc(-c4ccc5ccccc5c4)c4oc5ccccc5c34)n2)cc1 ; c1ccc(-c2nc(-c3ccc4c(c3)oc3ccccc34)nc(-c3ccc(-c4ccccc4)c4oc5ccccc5c34)n2)cc1 ; c1ccc(-c2nc(-c3ccc4c(c3)oc3ccccc34)nc(-c3cccc4oc5cccc(-c6ccccc6)c5c34)n2)cc1]

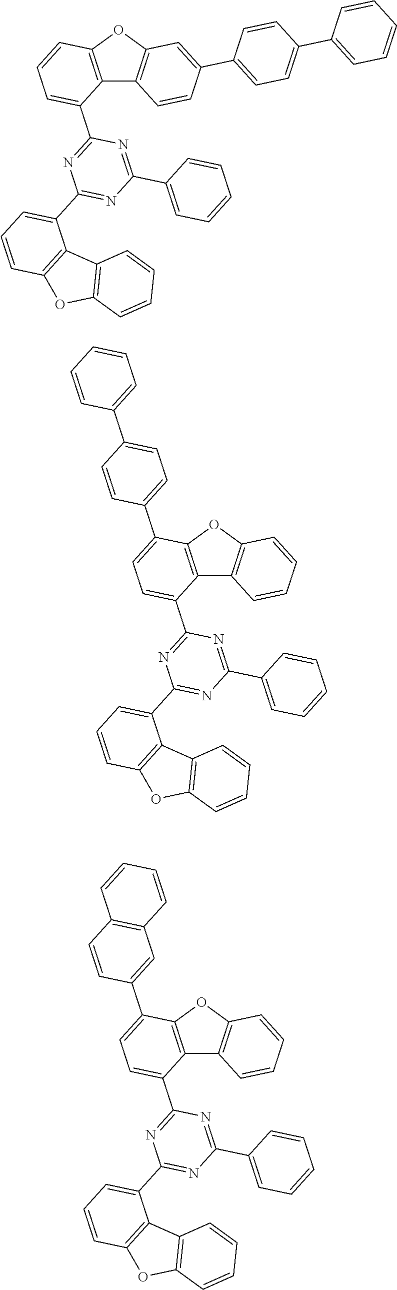

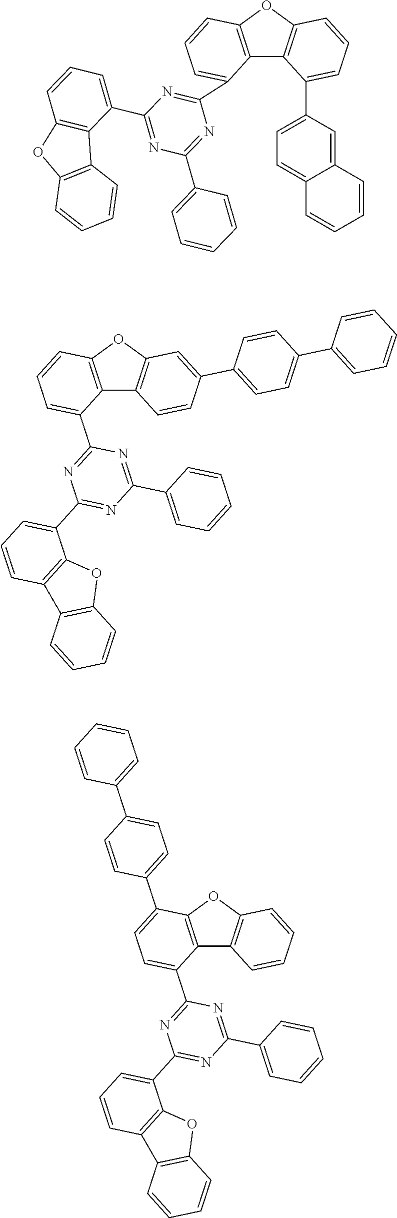 [SMILES: c1ccc(-c2nc(-c3cccc4oc5ccccc5c34)nc(-c3cccc4oc5cccc(-c6ccc7ccccc7c6)c5c34)n2)cc1 ; c1ccc(-c2ccc(-c3ccc4c(c3)oc3cccc(-c5nc(-c6ccccc6)nc(-c6cccc7c6oc6ccccc67)n5)c34)cc2)cc1 ; c1ccc(-c2ccc(-c3ccc(-c4nc(-c5ccccc5)nc(-c5cccc6c5oc5ccccc56)n4)c4c3oc3ccccc34)cc2)cc1]

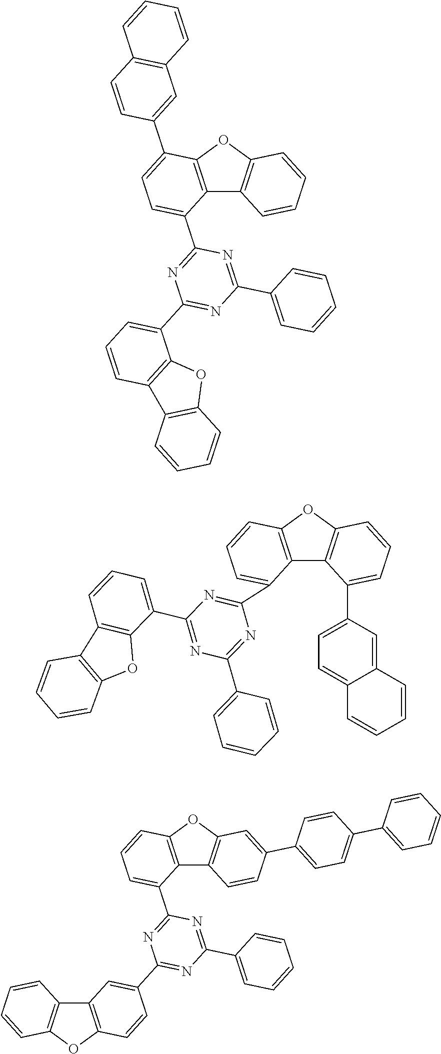 [SMILES: c1ccc(-c2nc(-c3cccc4c3oc3ccccc34)nc(-c3ccc(-c4ccc5ccccc5c4)c4oc5ccccc5c34)n2)cc1 ; c1ccc(-c2nc(-c3cccc4c3oc3ccccc34)nc(-c3cccc4oc5cccc(-c6ccc7ccccc7c6)c5c34)n2)cc1 ; c1ccc(-c2ccc(-c3ccc4c(c3)oc3cccc(-c5nc(-c6ccccc6)nc(-c6ccc7oc8ccccc8c7c6)n5)c34)cc2)cc1]

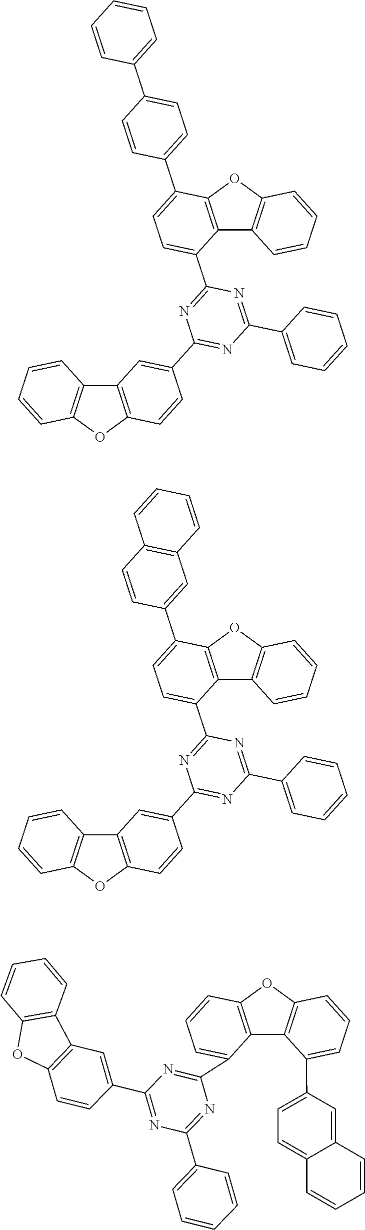 [SMILES: c1ccc(-c2ccc(-c3ccc(-c4nc(-c5ccccc5)nc(-c5ccc6oc7ccccc7c6c5)n4)c4c3oc3ccccc34)cc2)cc1 ; c1ccc(-c2nc(-c3ccc4oc5ccccc5c4c3)nc(-c3ccc(-c4ccc5ccccc5c4)c4oc5ccccc5c34)n2)cc1 ; c1ccc(-c2nc(-c3ccc4oc5ccccc5c4c3)nc(-c3cccc4oc5cccc(-c6ccc7ccccc7c6)c5c34)n2)cc1]

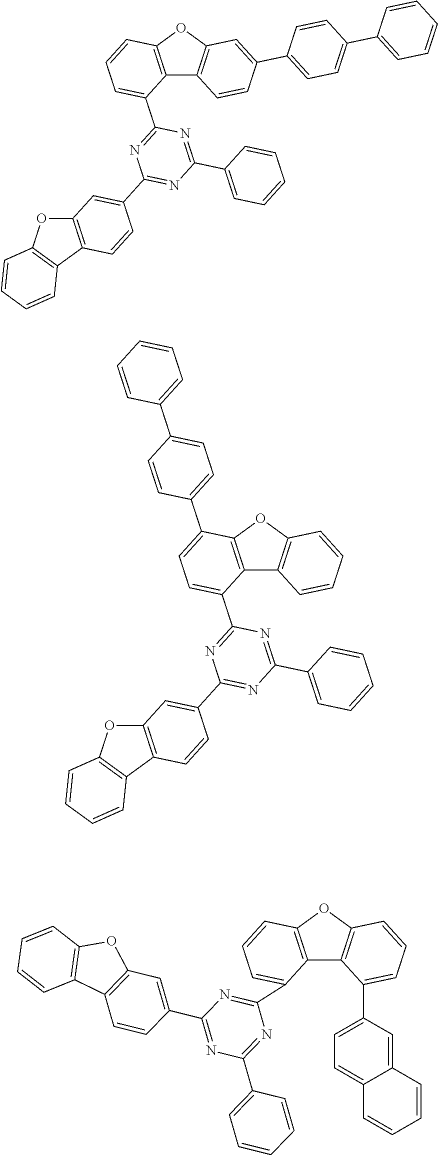 [SMILES: c1ccc(-c2ccc(-c3ccc4c(c3)oc3cccc(-c5nc(-c6ccccc6)nc(-c6ccc7c(c6)oc6ccccc67)n5)c34)cc2)cc1 ; c1ccc(-c2ccc(-c3ccc(-c4nc(-c5ccccc5)nc(-c5ccc6c(c5)oc5ccccc56)n4)c4c3oc3ccccc34)cc2)cc1 ; c1ccc(-c2nc(-c3ccc4c(c3)oc3ccccc34)nc(-c3cccc4oc5cccc(-c6ccc7ccccc7c6)c5c34)n2)cc1]

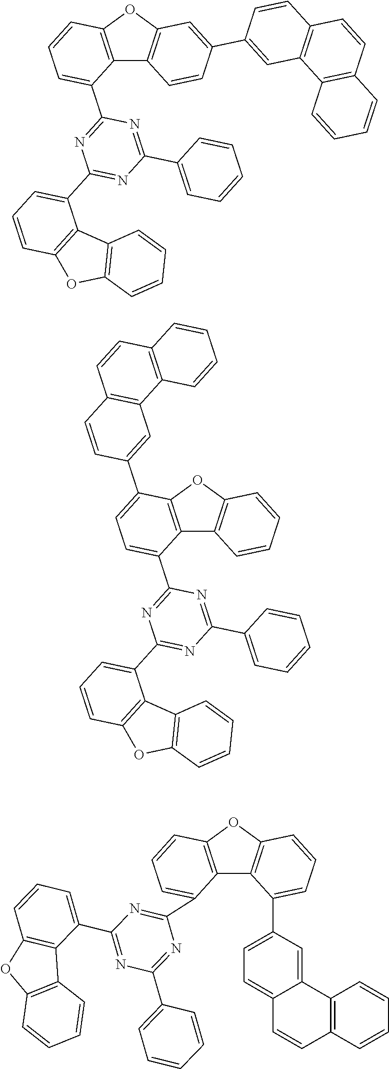

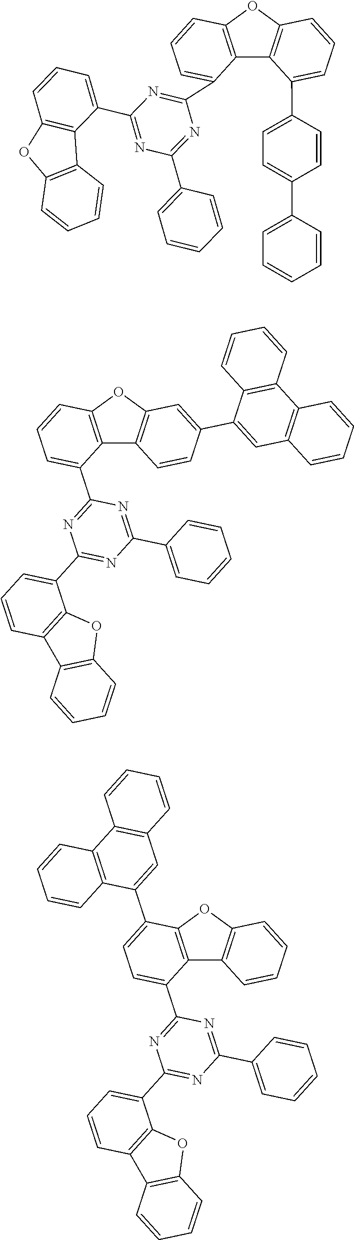 [SMILES: c1ccc(-c2ccc(-c3cccc4oc5cccc(-c6nc(-c7ccccc7)nc(-c7cccc8oc9ccccc9c78)n6)c5c34)cc2)cc1 ; c1ccc(-c2nc(-c3cccc4c3oc3ccccc34)nc(-c3cccc4oc5cc(-c6cc7ccccc7c7ccccc67)ccc5c34)n2)cc1 ; c1ccc(-c2nc(-c3cccc4c3oc3ccccc34)nc(-c3ccc(-c4cc5ccccc5c5ccccc45)c4oc5ccccc5c34)n2)cc1]

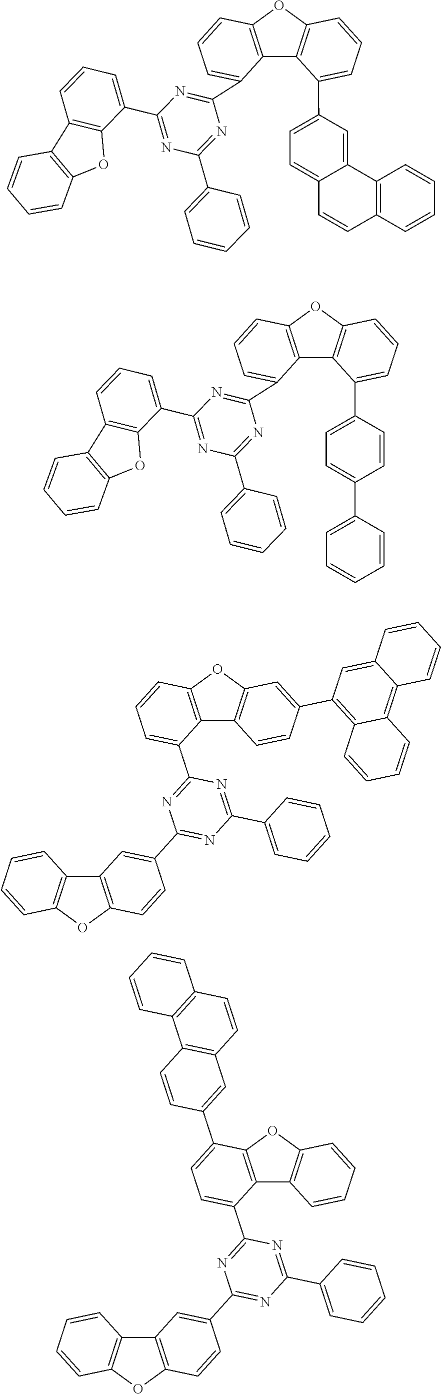

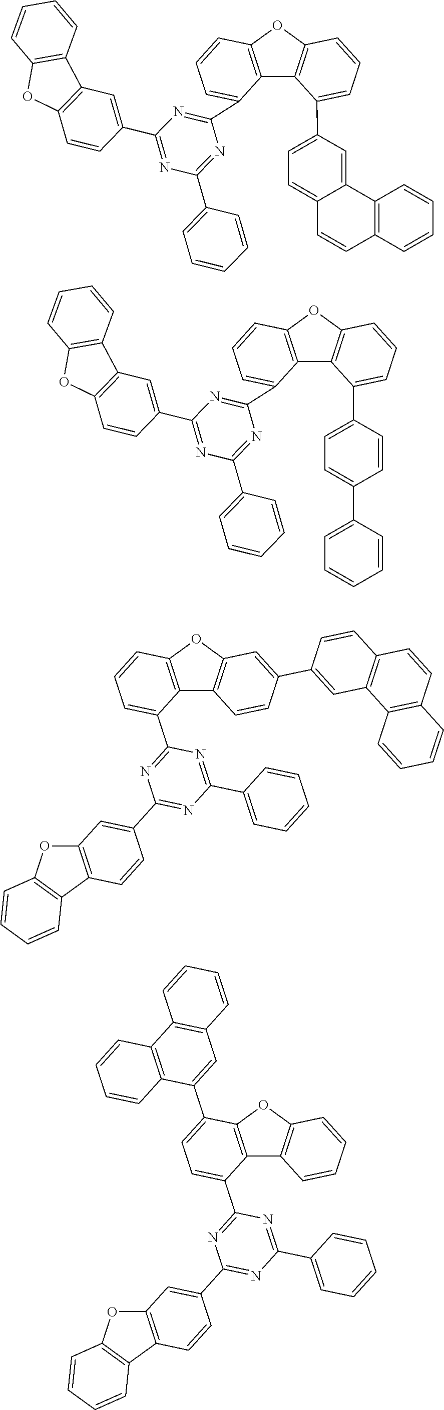

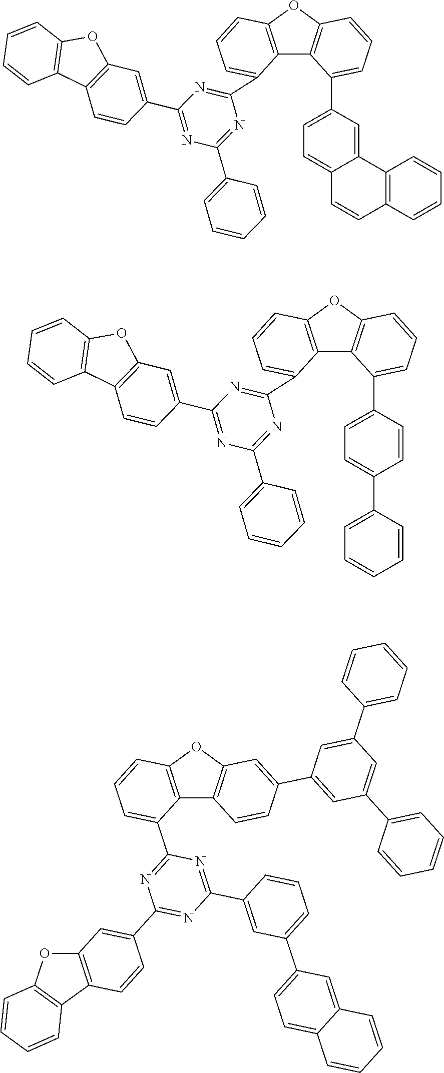 [SMILES: c1ccc(-c2nc(-c3ccc4c(c3)oc3ccccc34)nc(-c3cccc4oc5cccc(-c6ccc7ccc8ccccc8c7c6)c5c34)n2)cc1 ; c1ccc(-c2ccc(-c3cccc4oc5cccc(-c6nc(-c7ccccc7)nc(-c7ccc8c(c7)oc7ccccc78)n6)c5c34)cc2)cc1 ; c1ccc(-c2cc(-c3ccccc3)cc(-c3ccc4c(c3)oc3cccc(-c5nc(-c6cccc(-c7ccc8ccccc8c7)c6)nc(-c6ccc7c(c6)oc6ccccc67)n5)c34)c2)cc1]

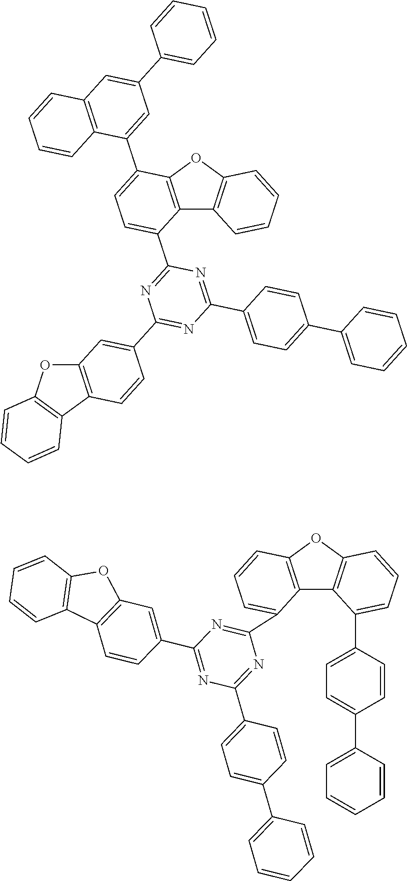 [SMILES: c1ccc(-c2ccc(-c3nc(-c4ccc5c(c4)oc4ccccc45)nc(-c4ccc(-c5cc(-c6ccccc6)cc6ccccc56)c5oc6ccccc6c45)n3)cc2)cc1 ; c1ccc(-c2ccc(-c3nc(-c4ccc5c(c4)oc4ccccc45)nc(-c4cccc5oc6cccc(-c7ccc(-c8ccccc8)cc7)c6c45)n3)cc2)cc1]

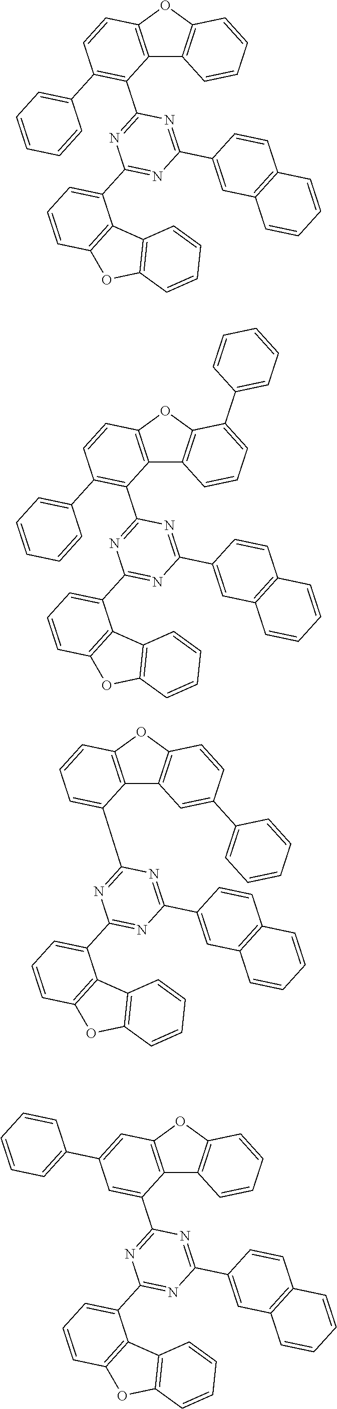

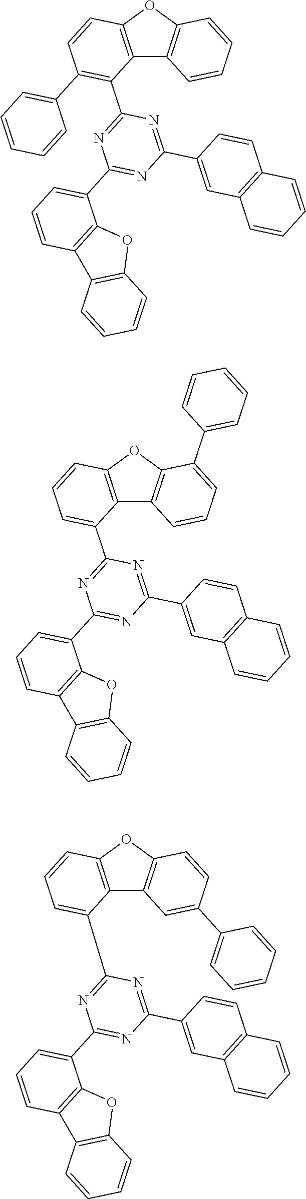 [SMILES: c1ccc(-c2ccc3oc4ccccc4c3c2-c2nc(-c3ccc4ccccc4c3)nc(-c3cccc4c3oc3ccccc34)n2)cc1 ; c1ccc(-c2cccc3c2oc2cccc(-c4nc(-c5ccc6ccccc6c5)nc(-c5cccc6c5oc5ccccc56)n4)c23)cc1 ; c1ccc(-c2ccc3oc4cccc(-c5nc(-c6ccc7ccccc7c6)nc(-c6cccc7c6oc6ccccc67)n5)c4c3c2)cc1]

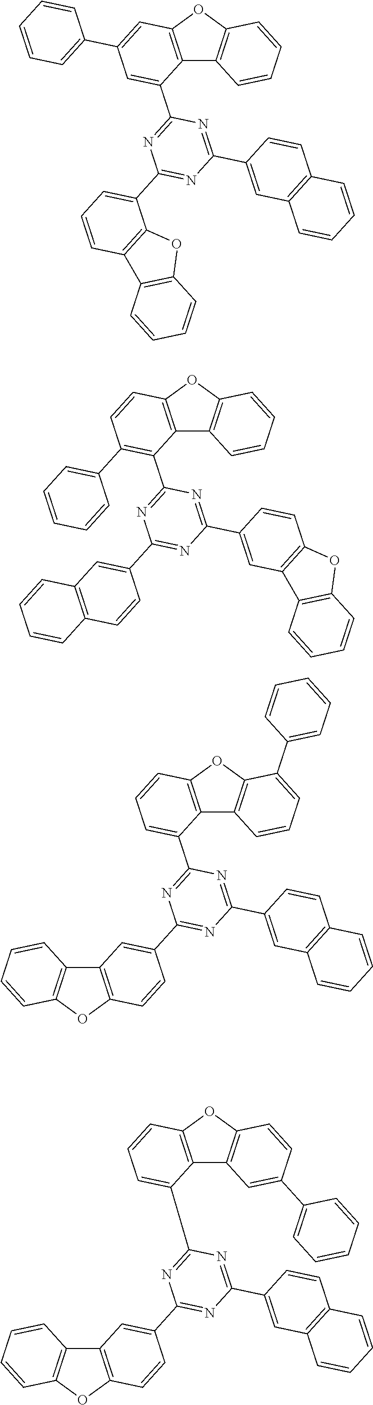

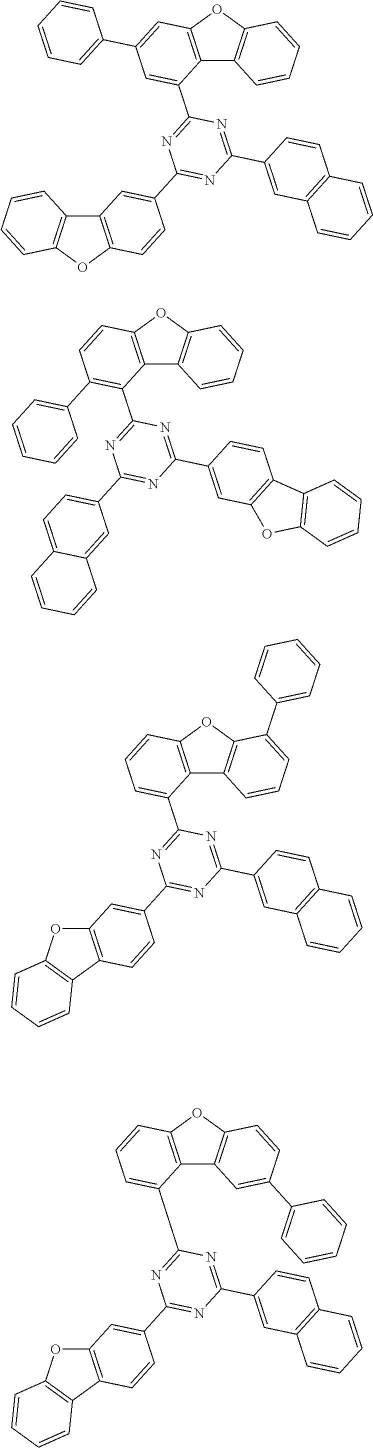 [SMILES: c1ccc(-c2cc(-c3nc(-c4ccc5ccccc5c4)nc(-c4ccc5oc6ccccc6c5c4)n3)c3c(c2)oc2ccccc23)cc1 ; c1ccc(-c2ccc3oc4ccccc4c3c2-c2nc(-c3ccc4ccccc4c3)nc(-c3ccc4c(c3)oc3ccccc34)n2)cc1 ; c1ccc(-c2cccc3c2oc2cccc(-c4nc(-c5ccc6ccccc6c5)nc(-c5ccc6c(c5)oc5ccccc56)n4)c23)cc1 ; c1ccc(-c2ccc3oc4cccc(-c5nc(-c6ccc7ccccc7c6)nc(-c6ccc7c(c6)oc6ccccc67)n5)c4c3c2)cc1]

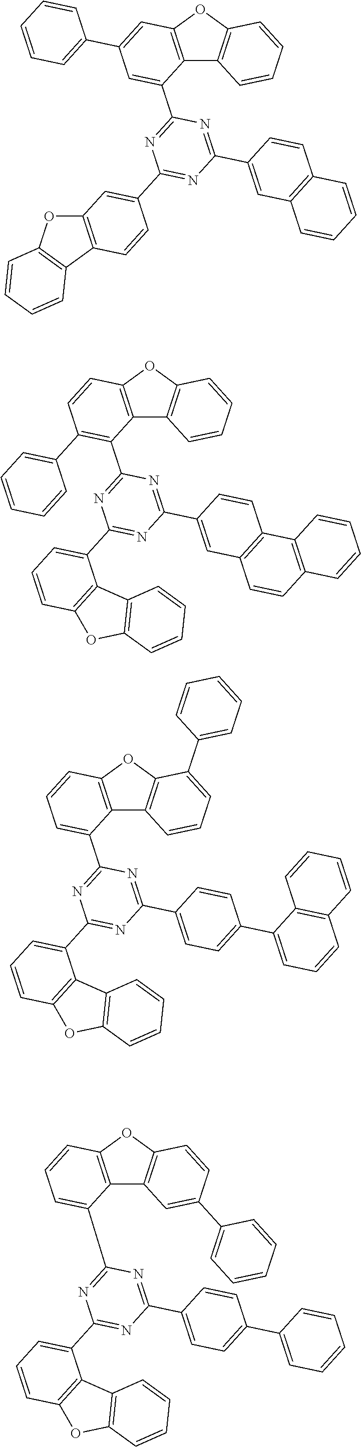

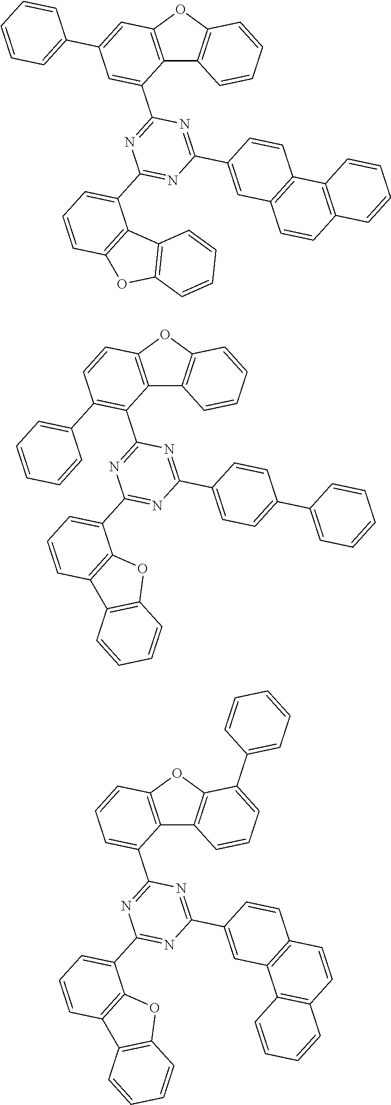

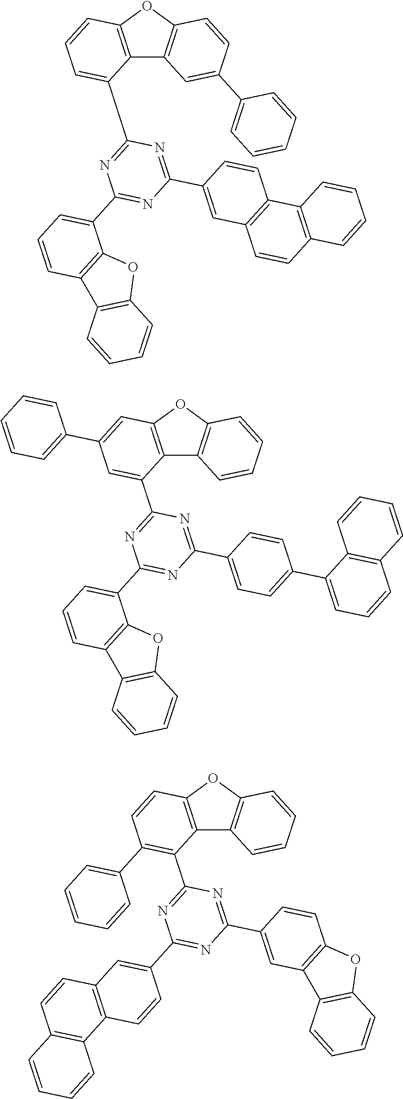 [SMILES: c1ccc(-c2ccc3oc4cccc(-c5nc(-c6ccc7c(ccc8ccccc87)c6)nc(-c6cccc7c6oc6ccccc67)n5)c4c3c2)cc1 ; c1ccc(-c2cc(-c3nc(-c4ccc(-c5cccc6ccccc56)cc4)nc(-c4cccc5c4oc4ccccc45)n3)c3c(c2)oc2ccccc23)cc1 ; c1ccc(-c2ccc3oc4ccccc4c3c2-c2nc(-c3ccc4c(ccc5ccccc54)c3)nc(-c3ccc4oc5ccccc5c4c3)n2)cc1]

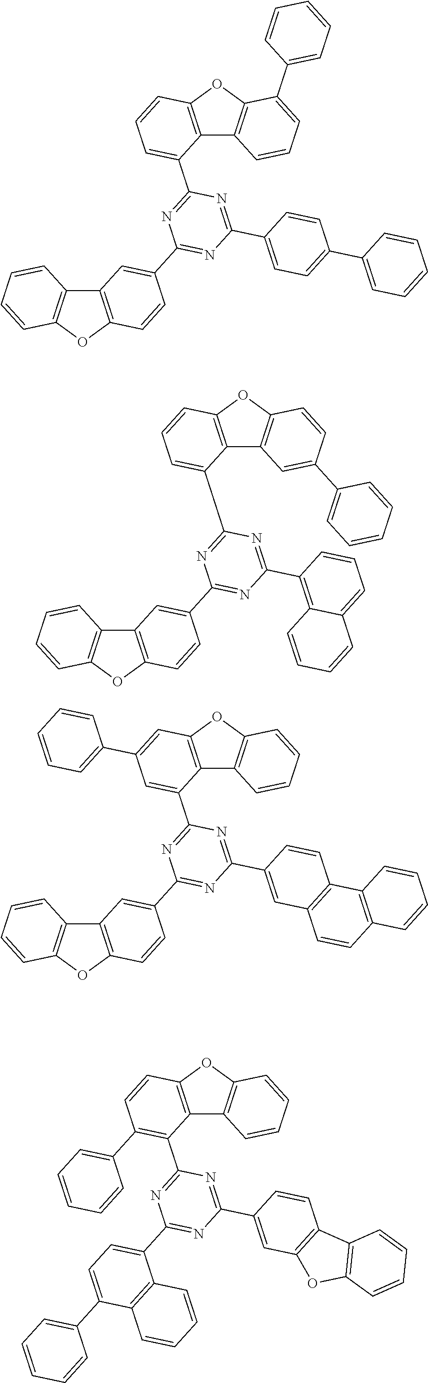

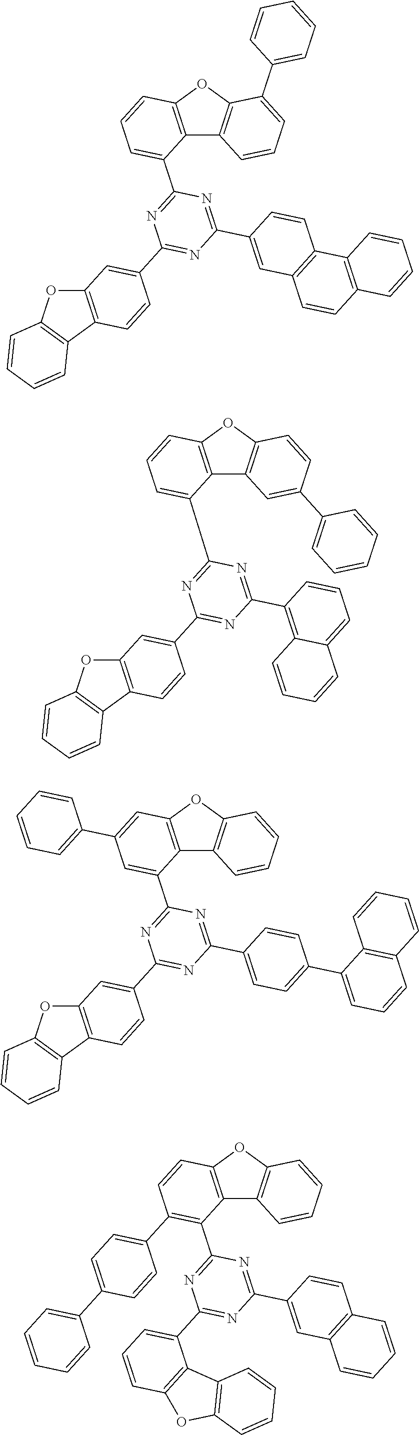

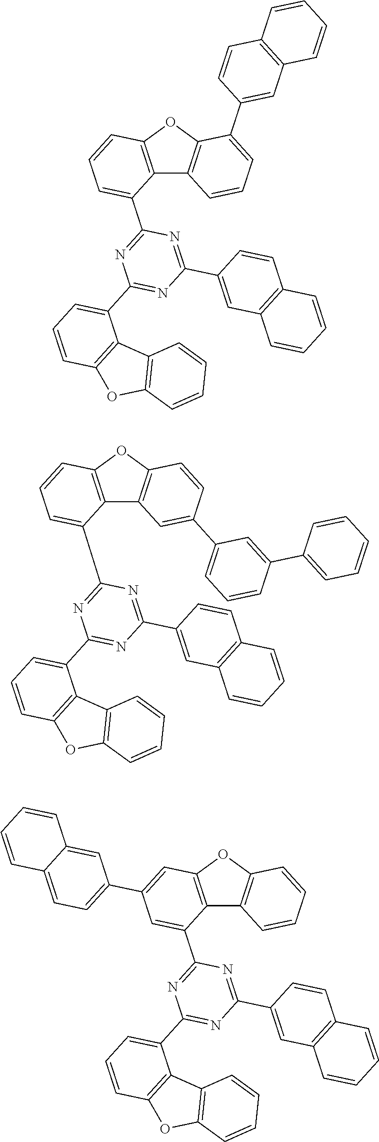 [SMILES: c1ccc2cc(-c3nc(-c4cccc5oc6ccccc6c45)nc(-c4cccc5oc6c(-c7ccc8ccccc8c7)cccc6c45)n3)ccc2c1 ; c1ccc(-c2cccc(-c3ccc4oc5cccc(-c6nc(-c7ccc8ccccc8c7)nc(-c7cccc8oc9ccccc9c78)n6)c5c4c3)c2)cc1 ; c1ccc2cc(-c3cc(-c4nc(-c5ccc6ccccc6c5)nc(-c5cccc6oc7ccccc7c56)n4)c4c(c3)oc3ccccc34)ccc2c1]

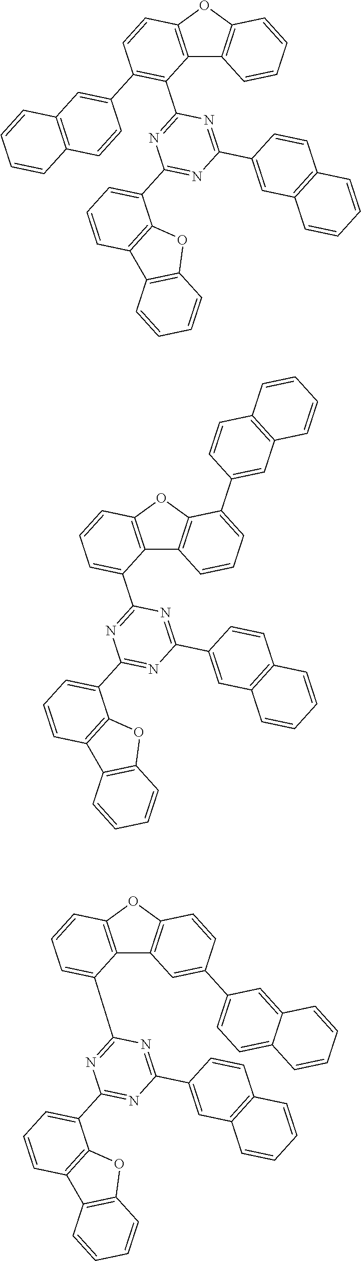 [SMILES: c1ccc2cc(-c3nc(-c4cccc5c4oc4ccccc45)nc(-c4c(-c5ccc6ccccc6c5)ccc5oc6ccccc6c45)n3)ccc2c1 ; c1ccc2cc(-c3nc(-c4cccc5c4oc4ccccc45)nc(-c4cccc5oc6c(-c7ccc8ccccc8c7)cccc6c45)n3)ccc2c1 ; c1ccc2cc(-c3ccc4oc5cccc(-c6nc(-c7ccc8ccccc8c7)nc(-c7cccc8c7oc7ccccc78)n6)c5c4c3)ccc2c1]

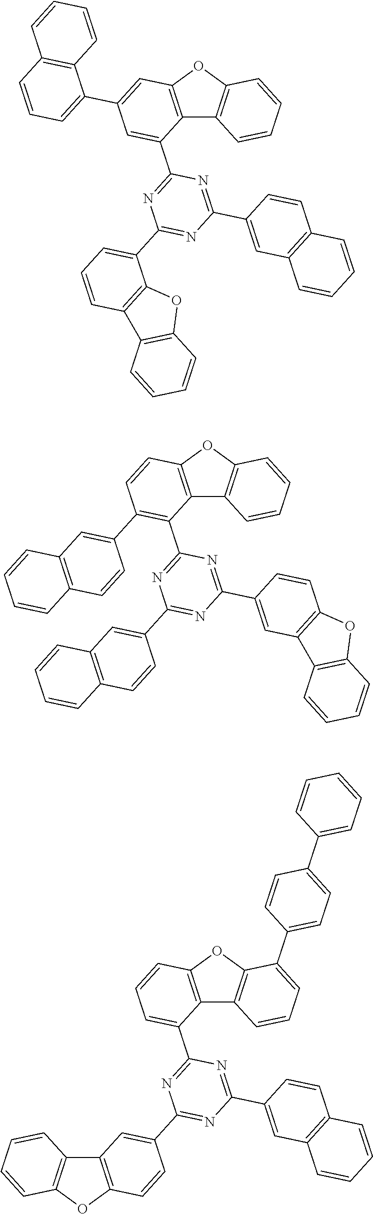 [SMILES: c1ccc2cc(-c3nc(-c4cccc5c4oc4ccccc45)nc(-c4cc(-c5cccc6ccccc56)cc5oc6ccccc6c45)n3)ccc2c1 ; c1ccc2cc(-c3nc(-c4ccc5oc6ccccc6c5c4)nc(-c4c(-c5ccc6ccccc6c5)ccc5oc6ccccc6c45)n3)ccc2c1 ; c1ccc(-c2ccc(-c3cccc4c3oc3cccc(-c5nc(-c6ccc7ccccc7c6)nc(-c6ccc7oc8ccccc8c7c6)n5)c34)cc2)cc1]

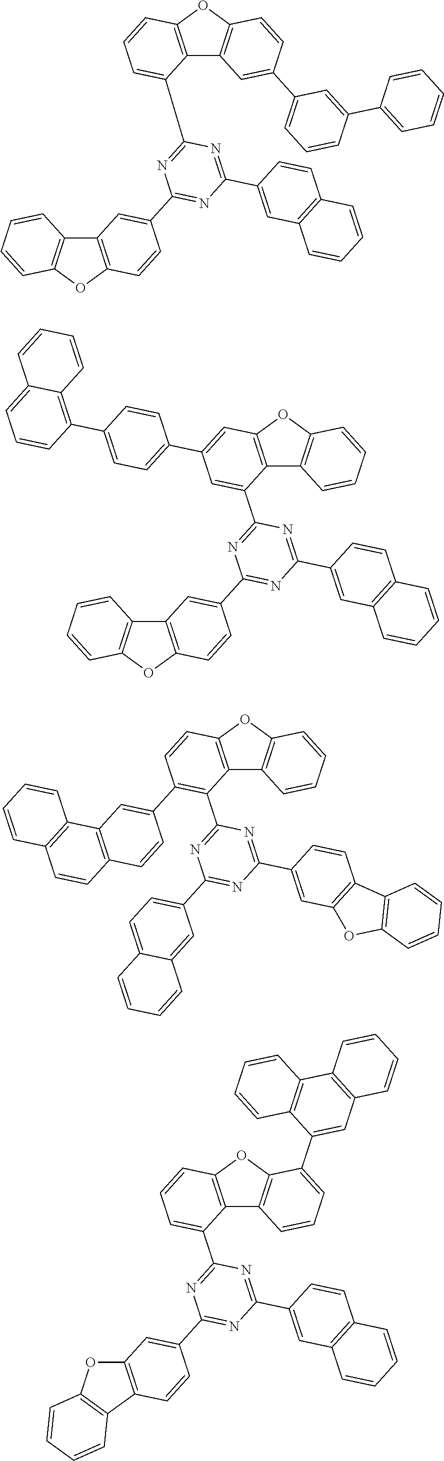 [SMILES: c1ccc(-c2cccc(-c3ccc4oc5cccc(-c6nc(-c7ccc8ccccc8c7)nc(-c7ccc8oc9ccccc9c8c7)n6)c5c4c3)c2)cc1 ; c1ccc2cc(-c3nc(-c4ccc5oc6ccccc6c5c4)nc(-c4cc(-c5ccc(-c6cccc7ccccc67)cc5)cc5oc6ccccc6c45)n3)ccc2c1 ; c1ccc2cc(-c3nc(-c4ccc5c(c4)oc4ccccc45)nc(-c4c(-c5ccc6ccc7ccccc7c6c5)ccc5oc6ccccc6c45)n3)ccc2c1 ; c1ccc2cc(-c3nc(-c4ccc5c(c4)oc4ccccc45)nc(-c4cccc5oc6c(-c7cc8ccccc8c8ccccc78)cccc6c45)n3)ccc2c1]

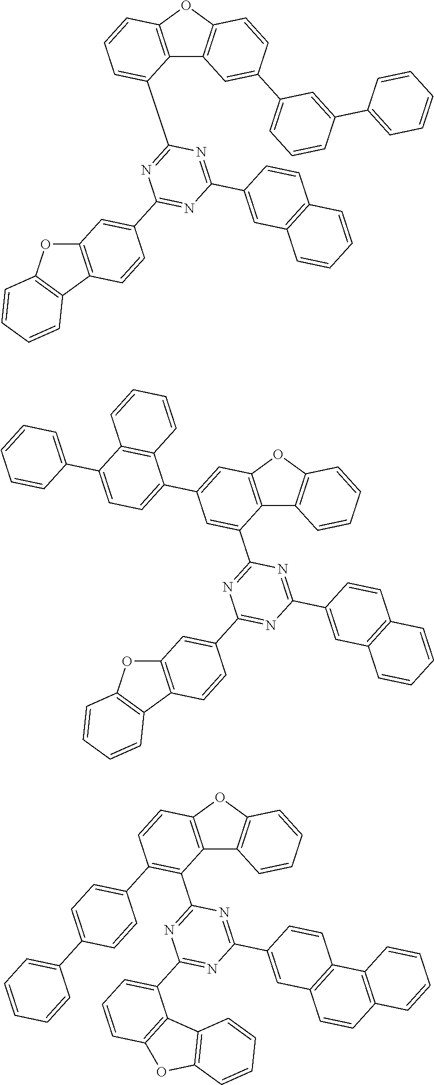 [SMILES: c1ccc(-c2cccc(-c3ccc4oc5cccc(-c6nc(-c7ccc8ccccc8c7)nc(-c7ccc8c(c7)oc7ccccc78)n6)c5c4c3)c2)cc1 ; c1ccc(-c2ccc(-c3cc(-c4nc(-c5ccc6ccccc6c5)nc(-c5ccc6c(c5)oc5ccccc56)n4)c4c(c3)oc3ccccc34)c3ccccc23)cc1 ; c1ccc(-c2ccc(-c3ccc4oc5ccccc5c4c3-c3nc(-c4ccc5c(ccc6ccccc65)c4)nc(-c4cccc5oc6ccccc6c45)n3)cc2)cc1]

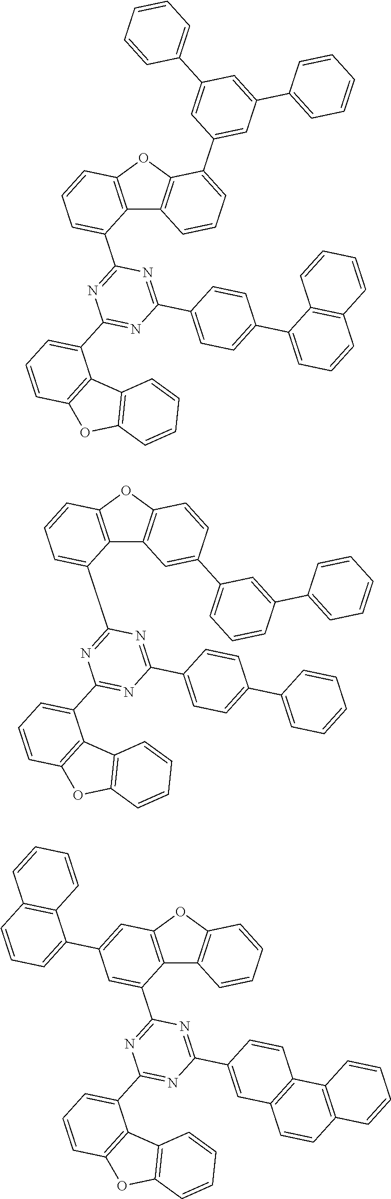 [SMILES: c1ccc(-c2cc(-c3ccccc3)cc(-c3cccc4c3oc3cccc(-c5nc(-c6ccc(-c7cccc8ccccc78)cc6)nc(-c6cccc7oc8ccccc8c67)n5)c34)c2)cc1 ; c1ccc(-c2ccc(-c3nc(-c4cccc5oc6ccccc6c45)nc(-c4cccc5oc6ccc(-c7cccc(-c8ccccc8)c7)cc6c45)n3)cc2)cc1 ; c1ccc2c(-c3cc(-c4nc(-c5ccc6c(ccc7ccccc76)c5)nc(-c5cccc6oc7ccccc7c56)n4)c4c(c3)oc3ccccc34)cccc2c1]

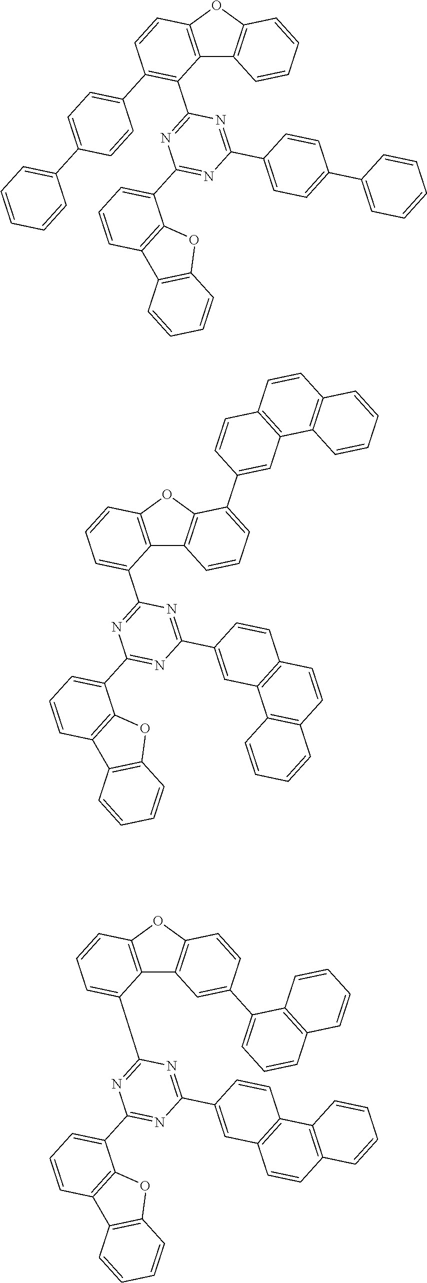 [SMILES: c1ccc(-c2ccc(-c3nc(-c4cccc5c4oc4ccccc45)nc(-c4c(-c5ccc(-c6ccccc6)cc5)ccc5oc6ccccc6c45)n3)cc2)cc1 ; c1ccc2c(c1)ccc1ccc(-c3nc(-c4cccc5c4oc4ccccc45)nc(-c4cccc5oc6c(-c7ccc8ccc9ccccc9c8c7)cccc6c45)n3)cc12 ; c1ccc2c(-c3ccc4oc5cccc(-c6nc(-c7ccc8c(ccc9ccccc98)c7)nc(-c7cccc8c7oc7ccccc78)n6)c5c4c3)cccc2c1]

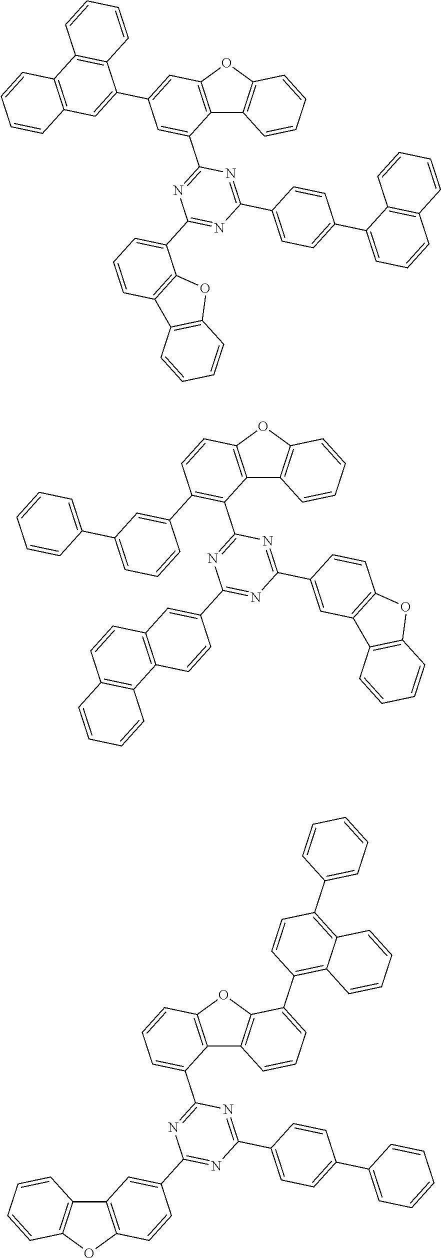 [SMILES: c1ccc2c(-c3ccc(-c4nc(-c5cccc6c5oc5ccccc56)nc(-c5cc(-c6cc7ccccc7c7ccccc67)cc6oc7ccccc7c56)n4)cc3)cccc2c1 ; c1ccc(-c2cccc(-c3ccc4oc5ccccc5c4c3-c3nc(-c4ccc5c(ccc6ccccc65)c4)nc(-c4ccc5oc6ccccc6c5c4)n3)c2)cc1 ; c1ccc(-c2ccc(-c3nc(-c4ccc5oc6ccccc6c5c4)nc(-c4cccc5oc6c(-c7ccc(-c8ccccc8)c8ccccc78)cccc6c45)n3)cc2)cc1]

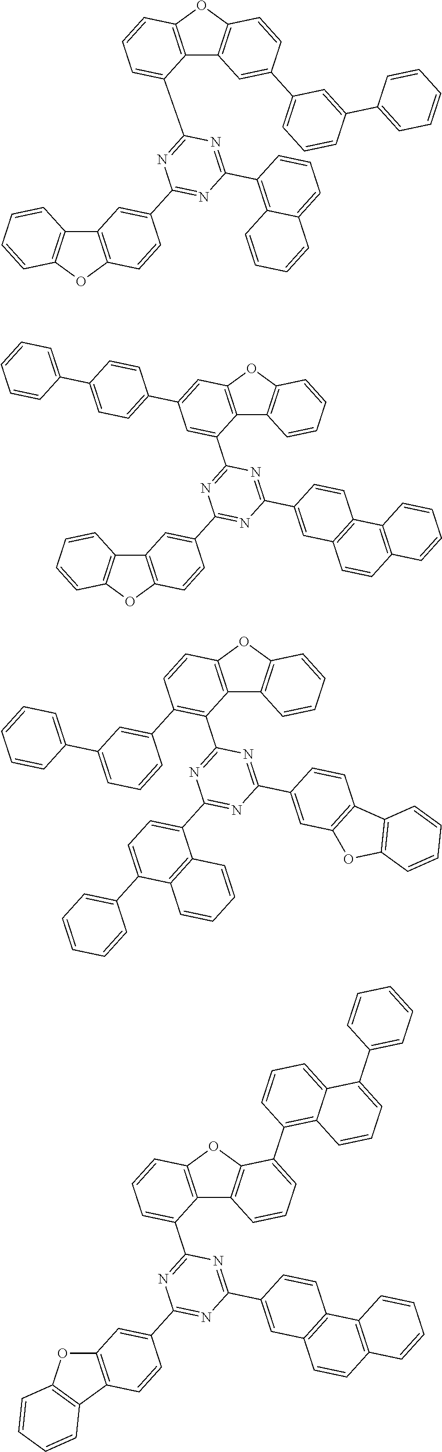 [SMILES: c1ccc(-c2cccc(-c3ccc4oc5cccc(-c6nc(-c7ccc8oc9ccccc9c8c7)nc(-c7cccc8ccccc78)n6)c5c4c3)c2)cc1 ; c1ccc(-c2ccc(-c3cc(-c4nc(-c5ccc6c(ccc7ccccc76)c5)nc(-c5ccc6oc7ccccc7c6c5)n4)c4c(c3)oc3ccccc34)cc2)cc1 ; c1ccc(-c2cccc(-c3ccc4oc5ccccc5c4c3-c3nc(-c4ccc5c(c4)oc4ccccc45)nc(-c4ccc(-c5ccccc5)c5ccccc45)n3)c2)cc1 ; c1ccc(-c2cccc3c(-c4cccc5c4oc4cccc(-c6nc(-c7ccc8c(ccc9ccccc98)c7)nc(-c7ccc8c(c7)oc7ccccc78)n6)c45)cccc23)cc1]

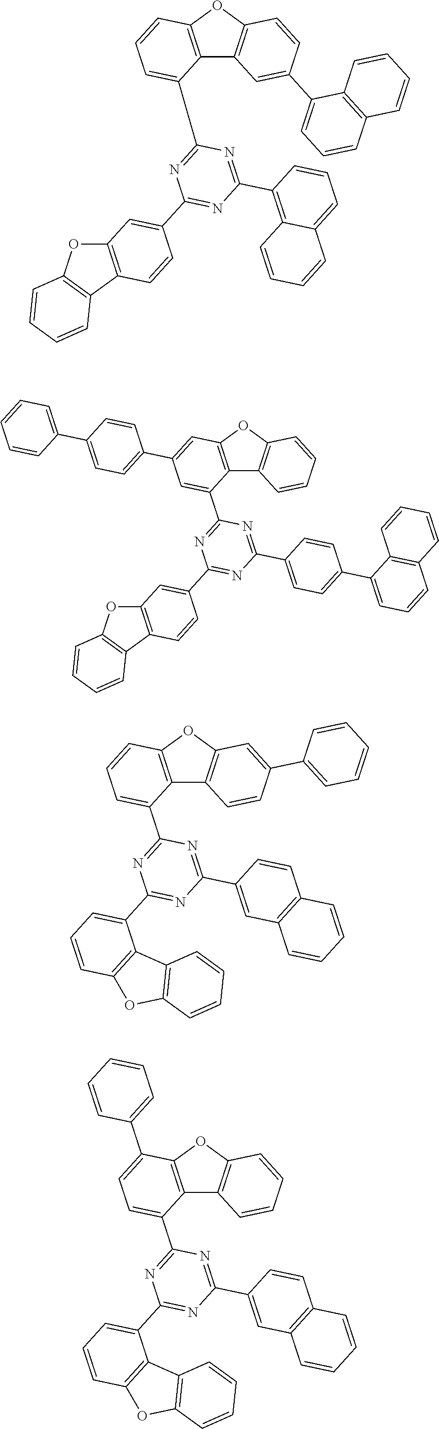 [SMILES: c1ccc2c(-c3ccc4oc5cccc(-c6nc(-c7ccc8c(c7)oc7ccccc78)nc(-c7cccc8ccccc78)n6)c5c4c3)cccc2c1 ; c1ccc(-c2ccc(-c3cc(-c4nc(-c5ccc(-c6cccc7ccccc67)cc5)nc(-c5ccc6c(c5)oc5ccccc56)n4)c4c(c3)oc3ccccc34)cc2)cc1 ; c1ccc(-c2ccc3c(c2)oc2cccc(-c4nc(-c5ccc6ccccc6c5)nc(-c5cccc6oc7ccccc7c56)n4)c23)cc1 ; c1ccc(-c2ccc(-c3nc(-c4ccc5ccccc5c4)nc(-c4cccc5oc6ccccc6c45)n3)c3c2oc2ccccc23)cc1]

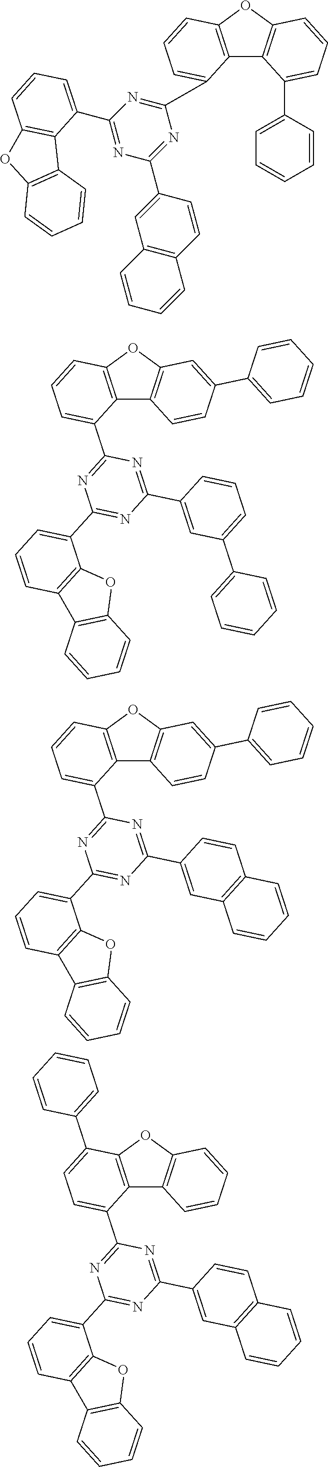

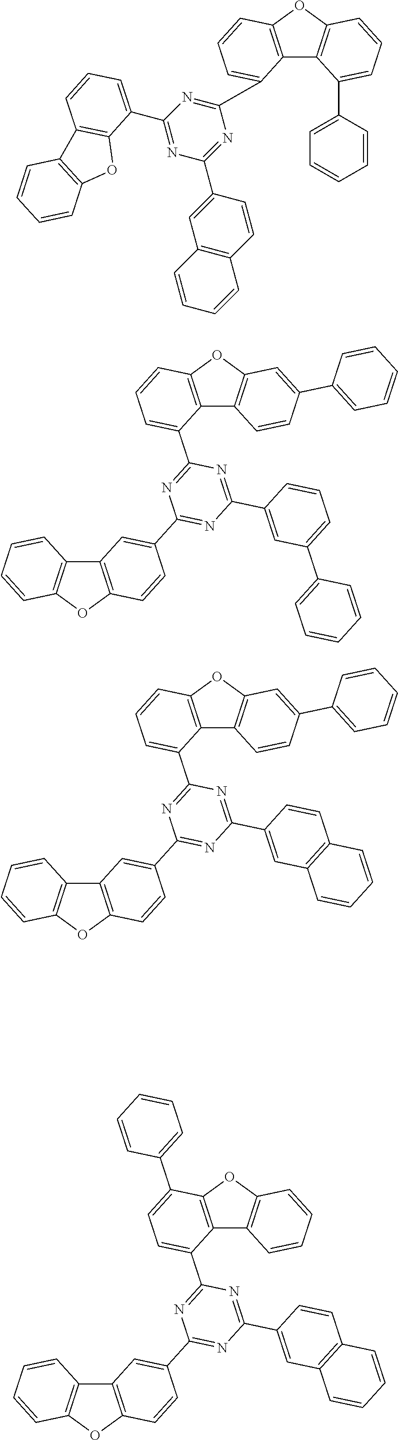

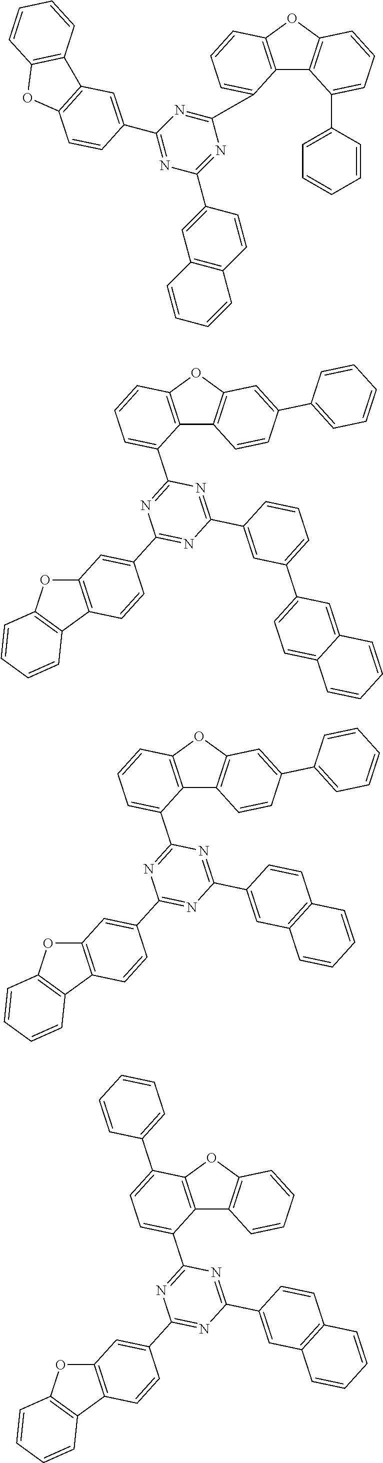

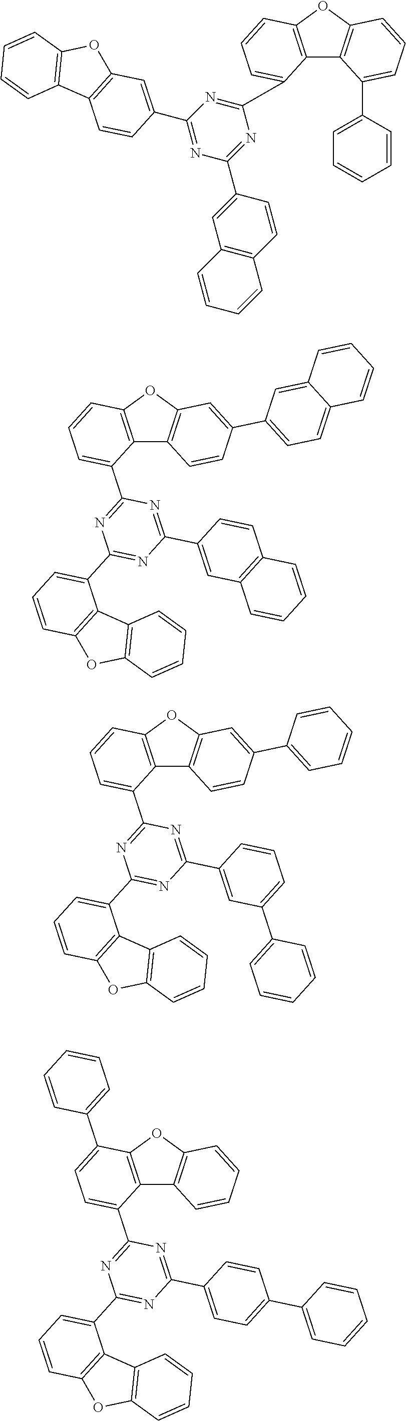

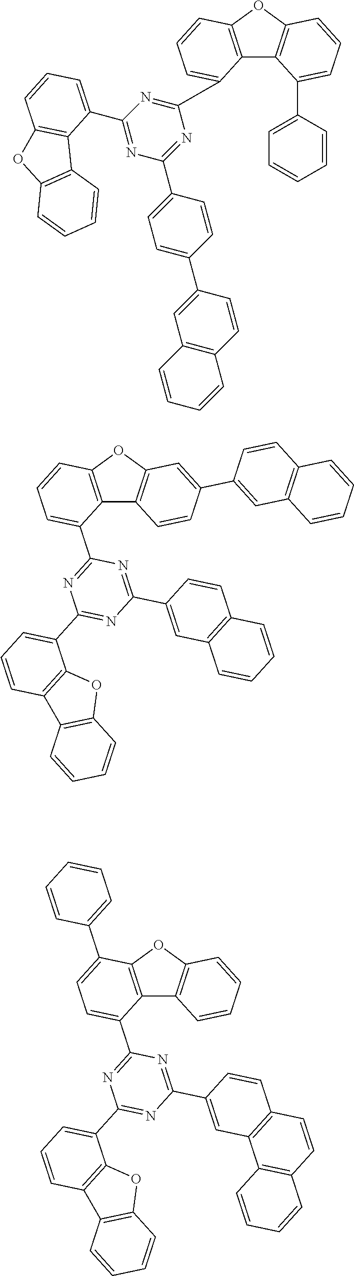 [SMILES: c1ccc(-c2cccc3oc4cccc(-c5nc(-c6ccc(-c7ccc8ccccc8c7)cc6)nc(-c6cccc7oc8ccccc8c67)n5)c4c23)cc1 ; c1ccc2cc(-c3ccc4c(c3)oc3cccc(-c5nc(-c6ccc7ccccc7c6)nc(-c6cccc7c6oc6ccccc67)n5)c34)ccc2c1 ; c1ccc(-c2ccc(-c3nc(-c4ccc5ccc6ccccc6c5c4)nc(-c4cccc5c4oc4ccccc45)n3)c3c2oc2ccccc23)cc1]

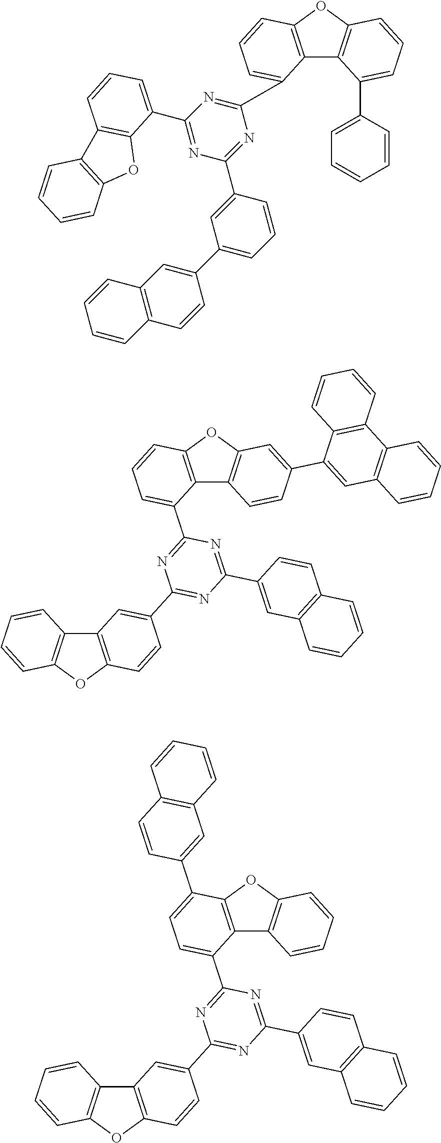 [SMILES: c1ccc(-c2cccc3oc4cccc(-c5nc(-c6cccc(-c7ccc8ccccc8c7)c6)nc(-c6cccc7c6oc6ccccc67)n5)c4c23)cc1 ; c1ccc2cc(-c3nc(-c4ccc5oc6ccccc6c5c4)nc(-c4cccc5oc6cc(-c7cc8ccccc8c8ccccc78)ccc6c45)n3)ccc2c1 ; c1ccc2cc(-c3nc(-c4ccc5oc6ccccc6c5c4)nc(-c4ccc(-c5ccc6ccccc6c5)c5oc6ccccc6c45)n3)ccc2c1]

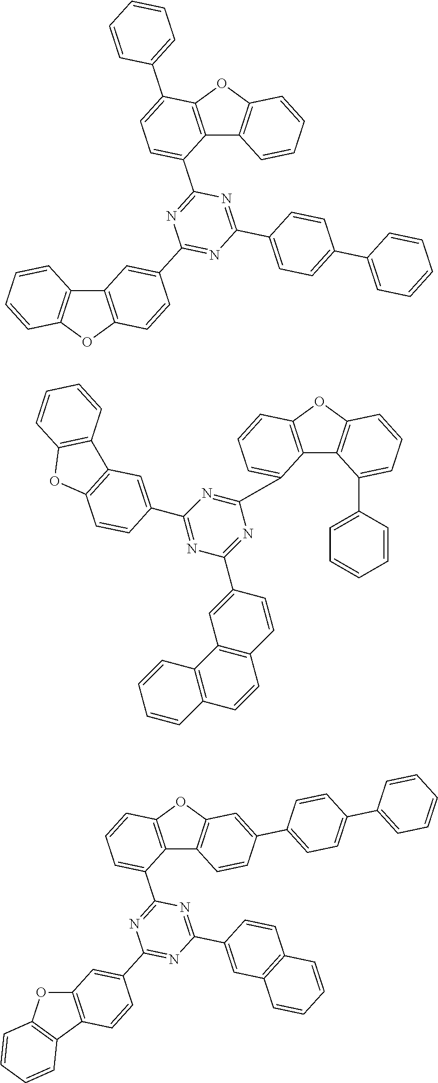

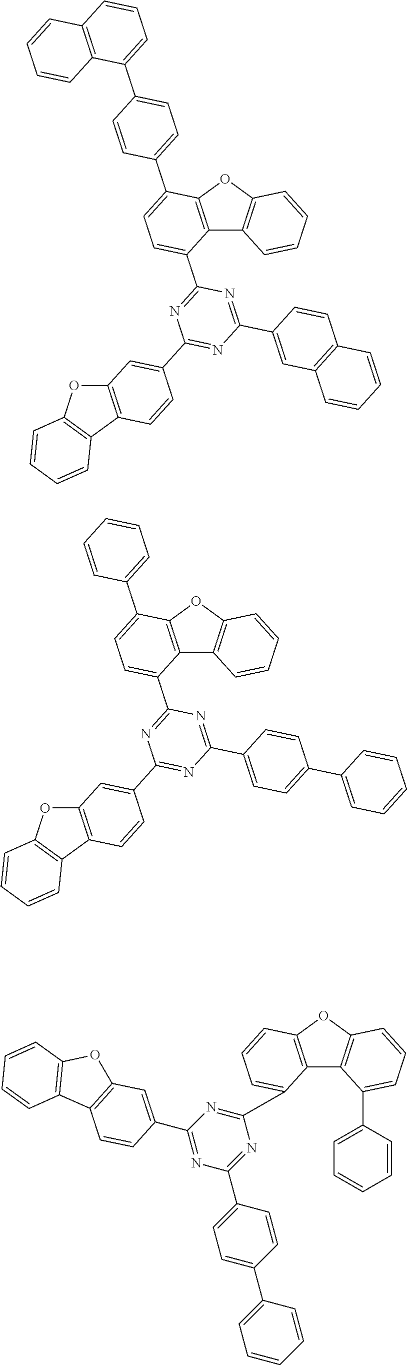 [SMILES: c1ccc2cc(-c3nc(-c4ccc5c(c4)oc4ccccc45)nc(-c4ccc(-c5ccc(-c6cccc7ccccc67)cc5)c5oc6ccccc6c45)n3)ccc2c1 ; c1ccc(-c2ccc(-c3nc(-c4ccc5c(c4)oc4ccccc45)nc(-c4ccc(-c5ccccc5)c5oc6ccccc6c45)n3)cc2)cc1 ; c1ccc(-c2ccc(-c3nc(-c4ccc5c(c4)oc4ccccc45)nc(-c4cccc5oc6cccc(-c7ccccc7)c6c45)n3)cc2)cc1]

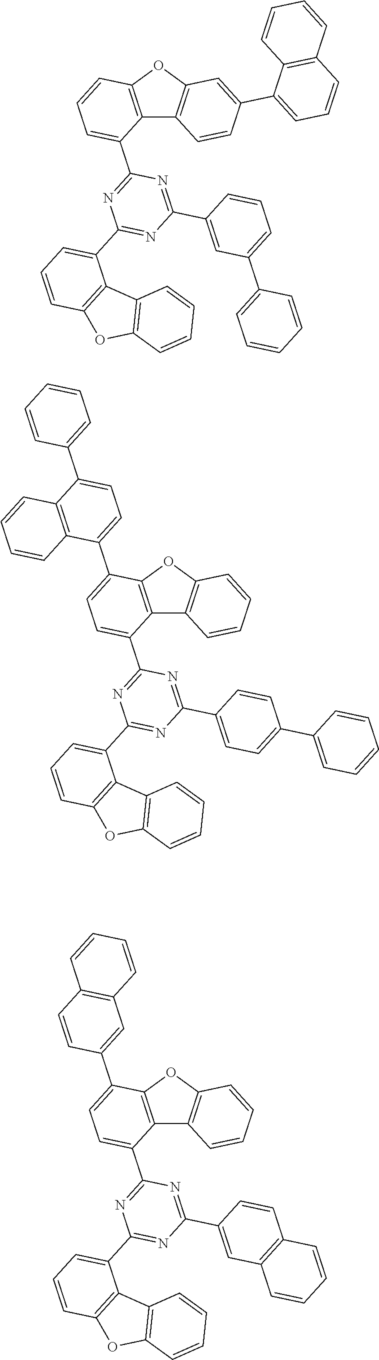 [SMILES: c1ccc(-c2cccc(-c3nc(-c4cccc5oc6ccccc6c45)nc(-c4cccc5oc6cc(-c7cccc8ccccc78)ccc6c45)n3)c2)cc1 ; c1ccc(-c2ccc(-c3nc(-c4cccc5oc6ccccc6c45)nc(-c4ccc(-c5ccc(-c6ccccc6)c6ccccc56)c5oc6ccccc6c45)n3)cc2)cc1 ; c1ccc2cc(-c3nc(-c4cccc5oc6ccccc6c45)nc(-c4ccc(-c5ccc6ccccc6c5)c5oc6ccccc6c45)n3)ccc2c1]

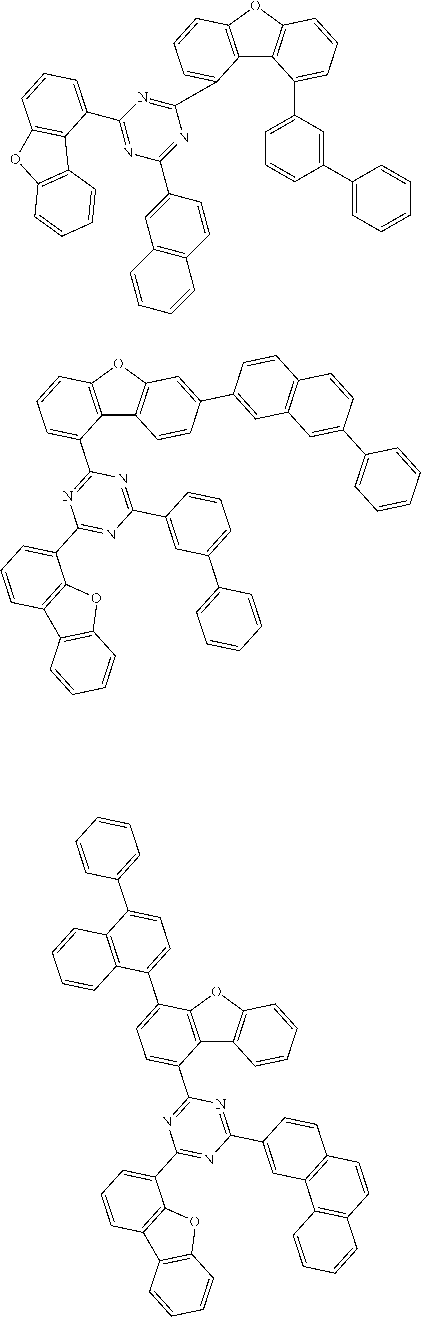 [SMILES: c1ccc(-c2cccc(-c3cccc4oc5cccc(-c6nc(-c7ccc8ccccc8c7)nc(-c7cccc8oc9ccccc9c78)n6)c5c34)c2)cc1 ; c1ccc(-c2cccc(-c3nc(-c4cccc5c4oc4ccccc45)nc(-c4cccc5oc6cc(-c7ccc8ccc(-c9ccccc9)cc8c7)ccc6c45)n3)c2)cc1 ; c1ccc(-c2ccc(-c3ccc(-c4nc(-c5ccc6ccc7ccccc7c6c5)nc(-c5cccc6c5oc5ccccc56)n4)c4c3oc3ccccc34)c3ccccc23)cc1]

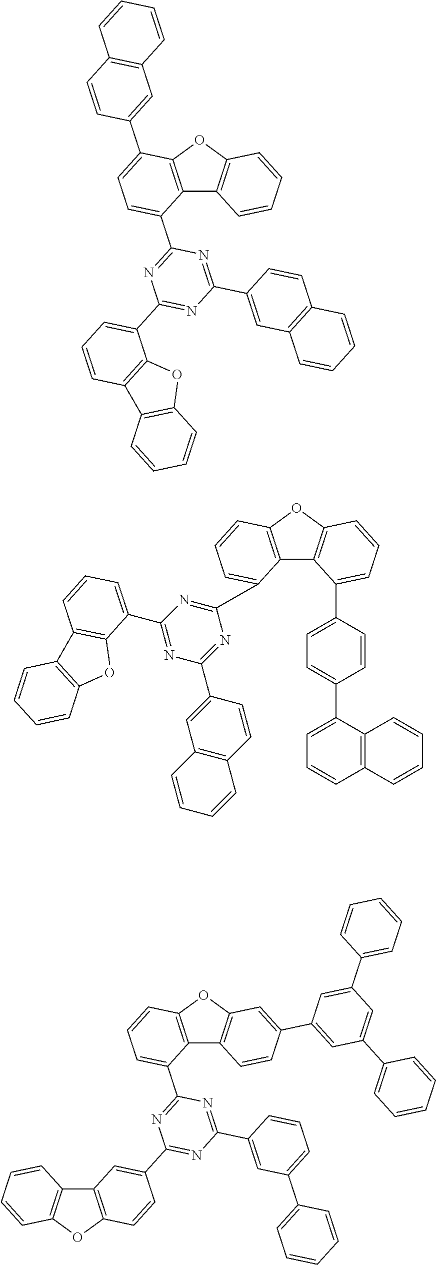 [SMILES: c1ccc2cc(-c3nc(-c4cccc5c4oc4ccccc45)nc(-c4ccc(-c5ccc6ccccc6c5)c5oc6ccccc6c45)n3)ccc2c1 ; c1ccc2cc(-c3nc(-c4cccc5c4oc4ccccc45)nc(-c4cccc5oc6cccc(-c7ccc(-c8cccc9ccccc89)cc7)c6c45)n3)ccc2c1 ; c1ccc(-c2cc(-c3ccccc3)cc(-c3ccc4c(c3)oc3cccc(-c5nc(-c6cccc(-c7ccccc7)c6)nc(-c6ccc7oc8ccccc8c7c6)n5)c34)c2)cc1]

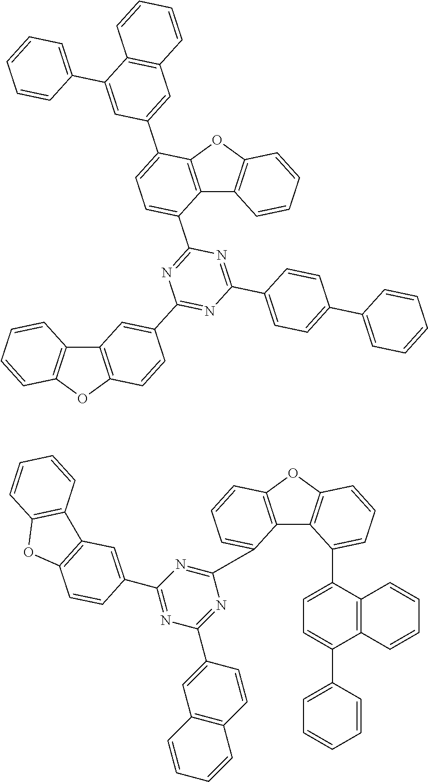 [SMILES: c1ccc(-c2ccc(-c3nc(-c4ccc5oc6ccccc6c5c4)nc(-c4ccc(-c5cc(-c6ccccc6)c6ccccc6c5)c5oc6ccccc6c45)n3)cc2)cc1 ; c1ccc(-c2ccc(-c3cccc4oc5cccc(-c6nc(-c7ccc8ccccc8c7)nc(-c7ccc8oc9ccccc9c8c7)n6)c5c34)c3ccccc23)cc1]

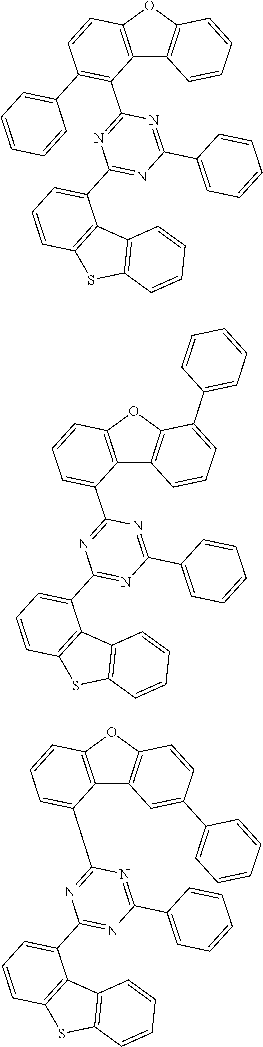

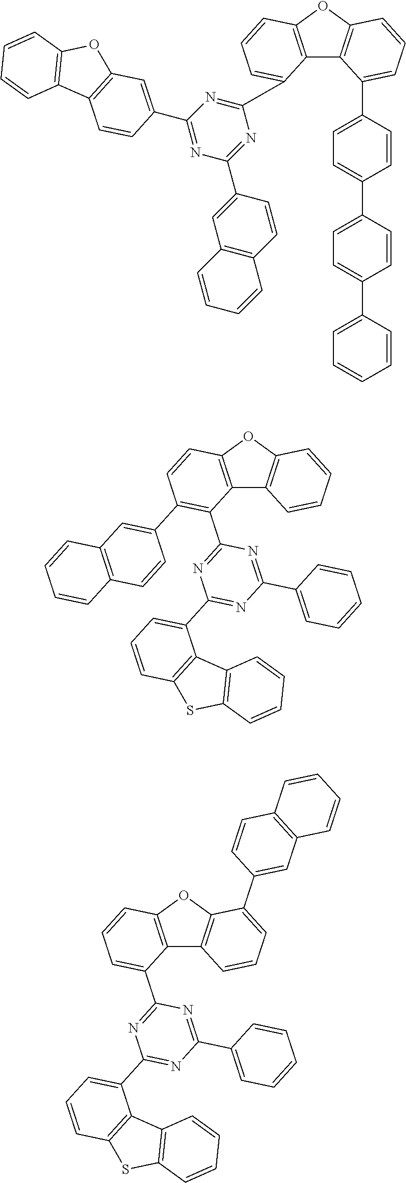 [SMILES: c1ccc(-c2ccc(-c3ccc(-c4cccc5oc6cccc(-c7nc(-c8ccc9ccccc9c8)nc(-c8ccc9c(c8)oc8ccccc89)n7)c6c45)cc3)cc2)cc1 ; c1ccc(-c2nc(-c3c(-c4ccc5ccccc5c4)ccc4oc5ccccc5c34)nc(-c3cccc4sc5ccccc5c34)n2)cc1 ; c1ccc(-c2nc(-c3cccc4oc5c(-c6ccc7ccccc7c6)cccc5c34)nc(-c3cccc4sc5ccccc5c34)n2)cc1]

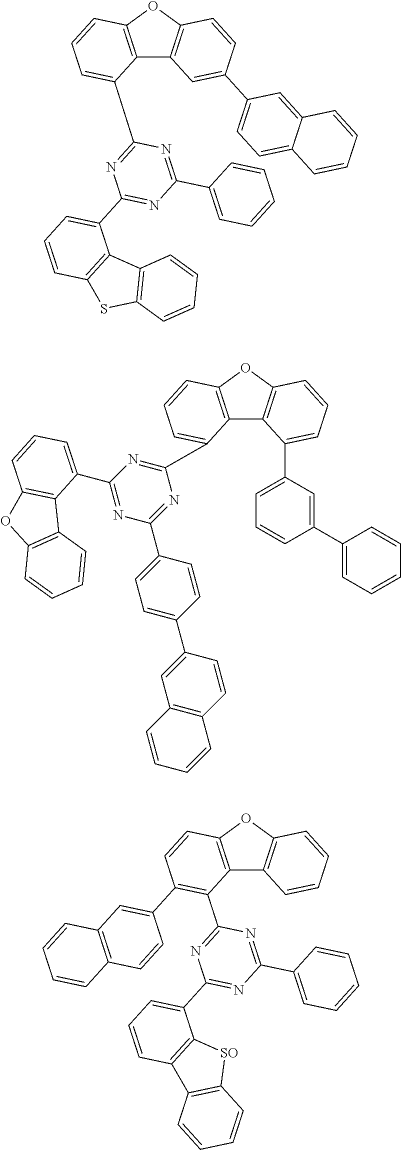

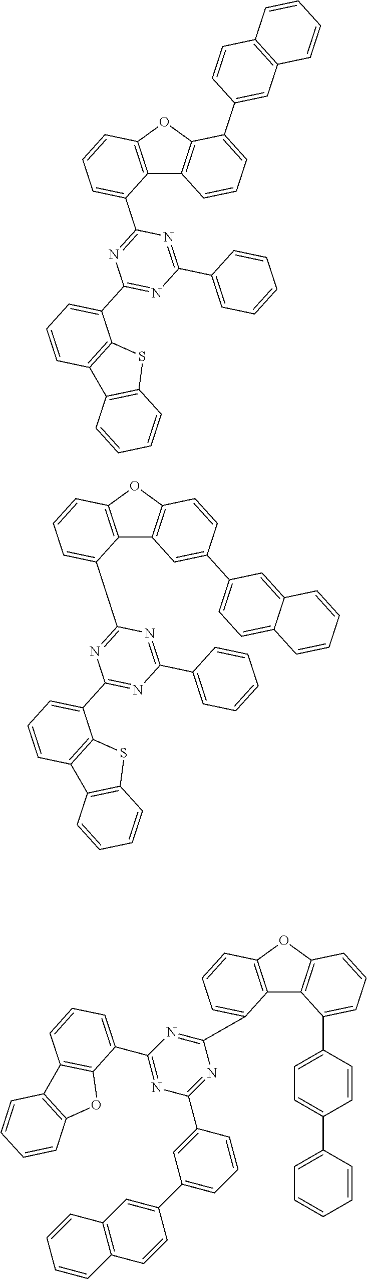

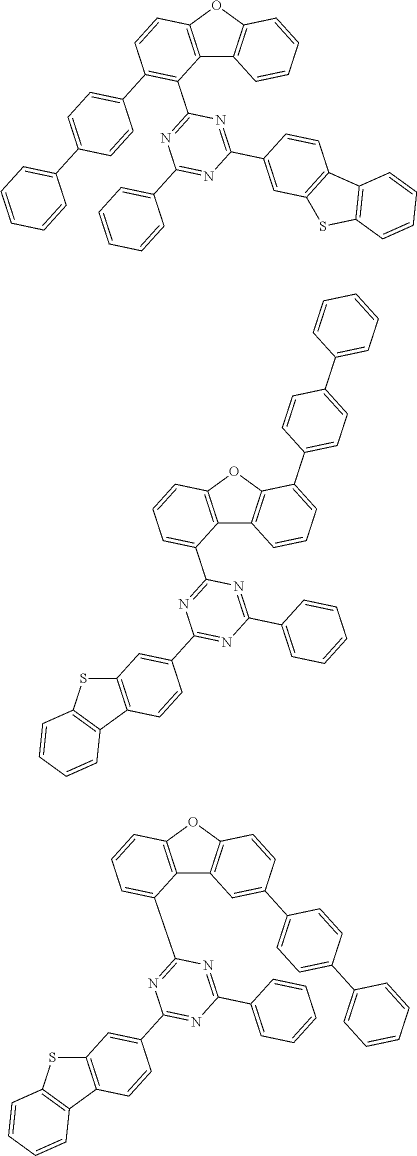 [SMILES: c1ccc(-c2ccc(-c3ccc4oc5ccccc5c4c3-c3nc(-c4ccccc4)nc(-c4ccc5c(c4)sc4ccccc45)n3)cc2)cc1 ; c1ccc(-c2ccc(-c3cccc4c3oc3cccc(-c5nc(-c6ccccc6)nc(-c6ccc7c(c6)sc6ccccc67)n5)c34)cc2)cc1 ; c1ccc(-c2ccc(-c3ccc4oc5cccc(-c6nc(-c7ccccc7)nc(-c7ccc8c(c7)sc7ccccc78)n6)c5c4c3)cc2)cc1]

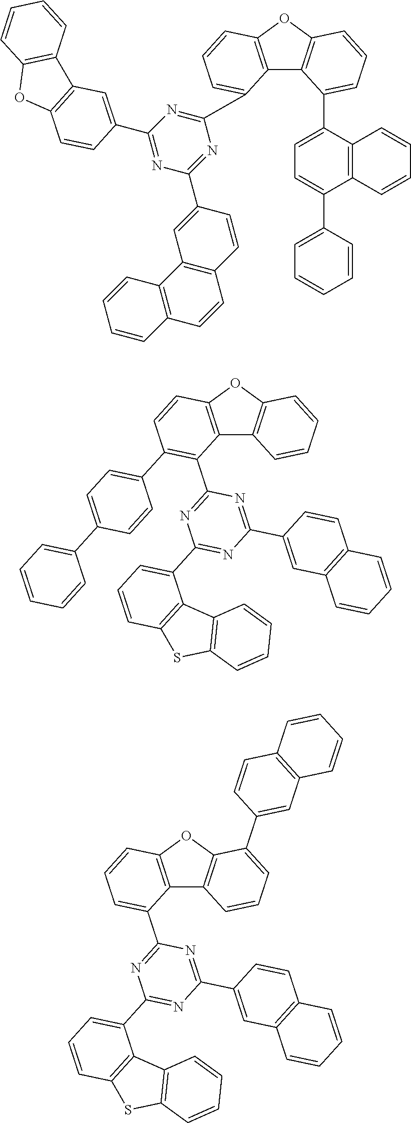 [SMILES: c1ccc(-c2ccc(-c3cccc4oc5cccc(-c6nc(-c7ccc8ccc9ccccc9c8c7)nc(-c7ccc8oc9ccccc9c8c7)n6)c5c34)c3ccccc23)cc1 ; c1ccc(-c2ccc(-c3ccc4oc5ccccc5c4c3-c3nc(-c4ccc5ccccc5c4)nc(-c4cccc5sc6ccccc6c45)n3)cc2)cc1 ; c1ccc2cc(-c3nc(-c4cccc5oc6c(-c7ccc8ccccc8c7)cccc6c45)nc(-c4cccc5sc6ccccc6c45)n3)ccc2c1]

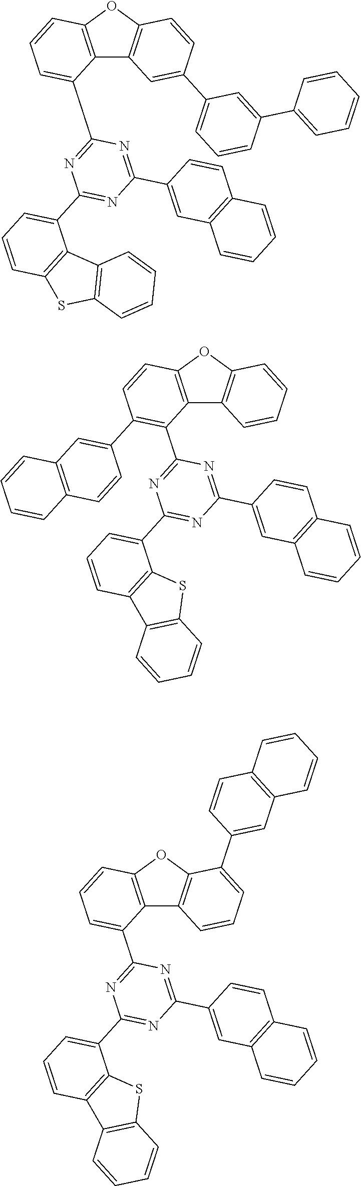 [SMILES: c1ccc(-c2cccc(-c3ccc4oc5cccc(-c6nc(-c7ccc8ccccc8c7)nc(-c7cccc8sc9ccccc9c78)n6)c5c4c3)c2)cc1 ; c1ccc2cc(-c3nc(-c4cccc5c4sc4ccccc45)nc(-c4c(-c5ccc6ccccc6c5)ccc5oc6ccccc6c45)n3)ccc2c1 ; c1ccc2cc(-c3nc(-c4cccc5c4sc4ccccc45)nc(-c4cccc5oc6c(-c7ccc8ccccc8c7)cccc6c45)n3)ccc2c1]

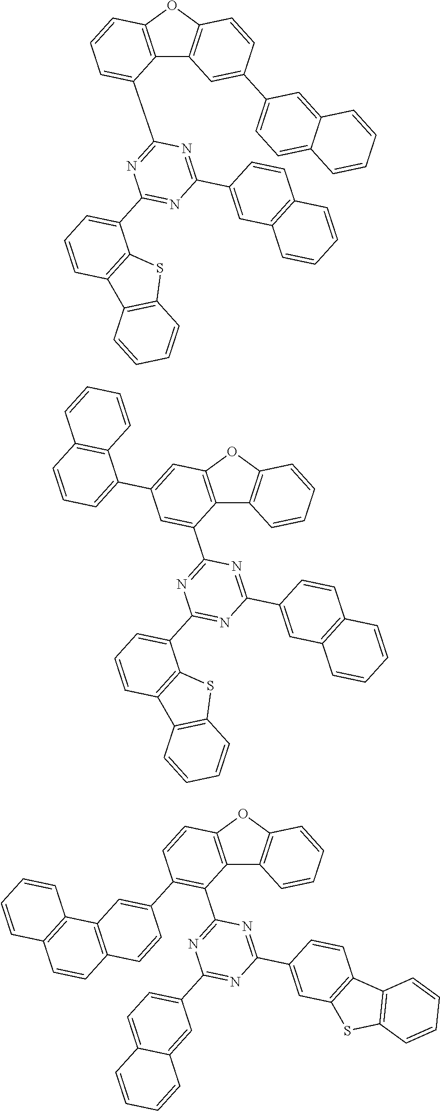

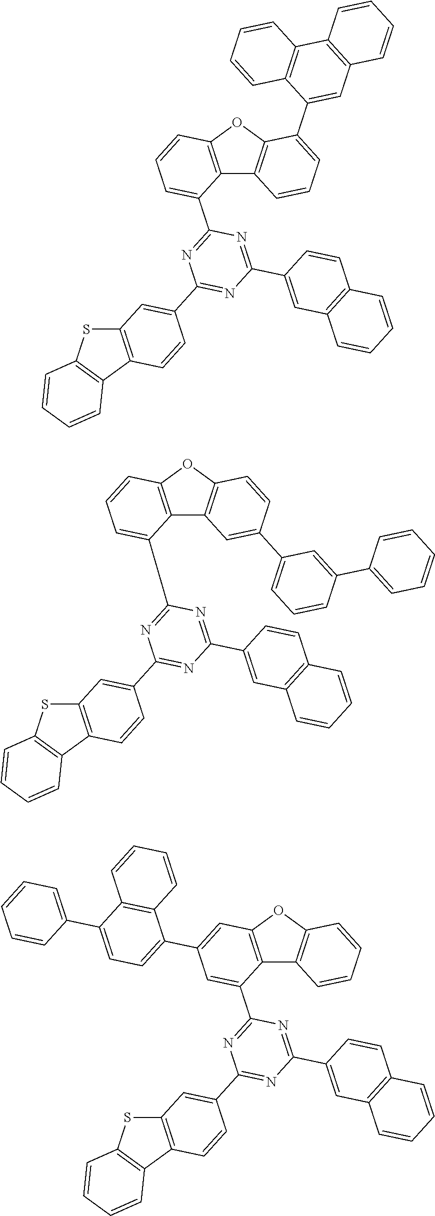 [SMILES: c1ccc2cc(-c3nc(-c4ccc5c(c4)sc4ccccc45)nc(-c4cccc5oc6c(-c7cc8ccccc8c8ccccc78)cccc6c45)n3)ccc2c1 ; c1ccc(-c2cccc(-c3ccc4oc5cccc(-c6nc(-c7ccc8ccccc8c7)nc(-c7ccc8c(c7)sc7ccccc78)n6)c5c4c3)c2)cc1 ; c1ccc(-c2ccc(-c3cc(-c4nc(-c5ccc6ccccc6c5)nc(-c5ccc6c(c5)sc5ccccc56)n4)c4c(c3)oc3ccccc34)c3ccccc23)cc1]

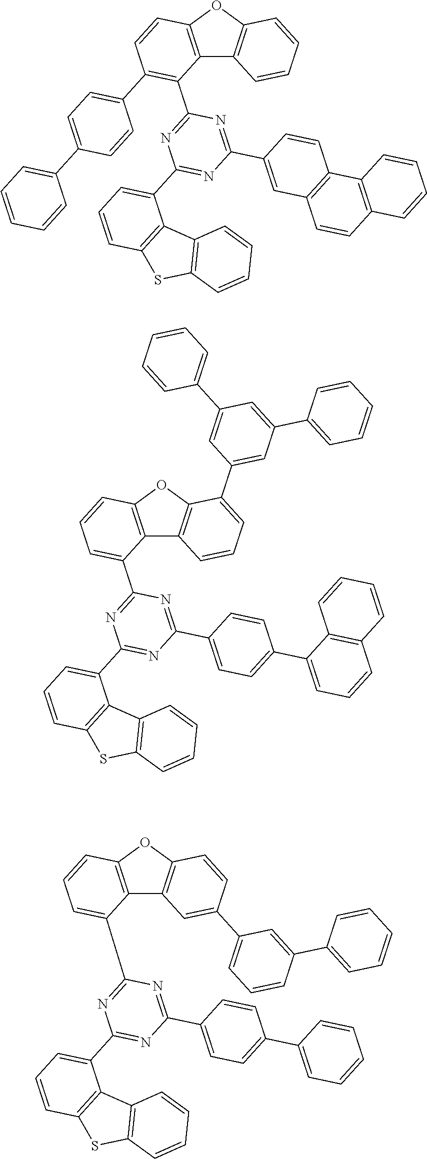 [SMILES: c1ccc(-c2ccc(-c3ccc4oc5ccccc5c4c3-c3nc(-c4ccc5c(ccc6ccccc65)c4)nc(-c4cccc5sc6ccccc6c45)n3)cc2)cc1 ; c1ccc(-c2cc(-c3ccccc3)cc(-c3cccc4c3oc3cccc(-c5nc(-c6ccc(-c7cccc8ccccc78)cc6)nc(-c6cccc7sc8ccccc8c67)n5)c34)c2)cc1 ; c1ccc(-c2ccc(-c3nc(-c4cccc5oc6ccc(-c7cccc(-c8ccccc8)c7)cc6c45)nc(-c4cccc5sc6ccccc6c45)n3)cc2)cc1]

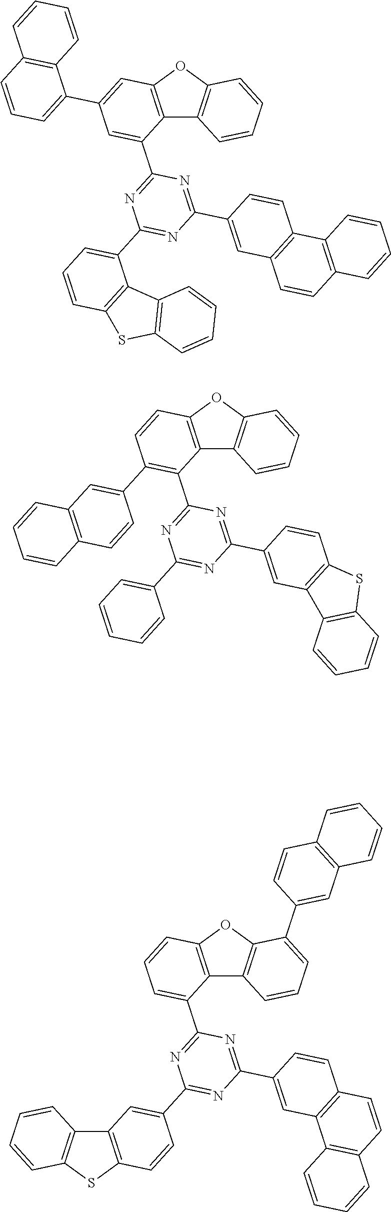 [SMILES: c1ccc2c(-c3cc(-c4nc(-c5ccc6c(ccc7ccccc76)c5)nc(-c5cccc6sc7ccccc7c56)n4)c4c(c3)oc3ccccc34)cccc2c1 ; c1ccc(-c2nc(-c3ccc4sc5ccccc5c4c3)nc(-c3c(-c4ccc5ccccc5c4)ccc4oc5ccccc5c34)n2)cc1 ; c1ccc2cc(-c3cccc4c3oc3cccc(-c5nc(-c6ccc7ccc8ccccc8c7c6)nc(-c6ccc7sc8ccccc8c7c6)n5)c34)ccc2c1]

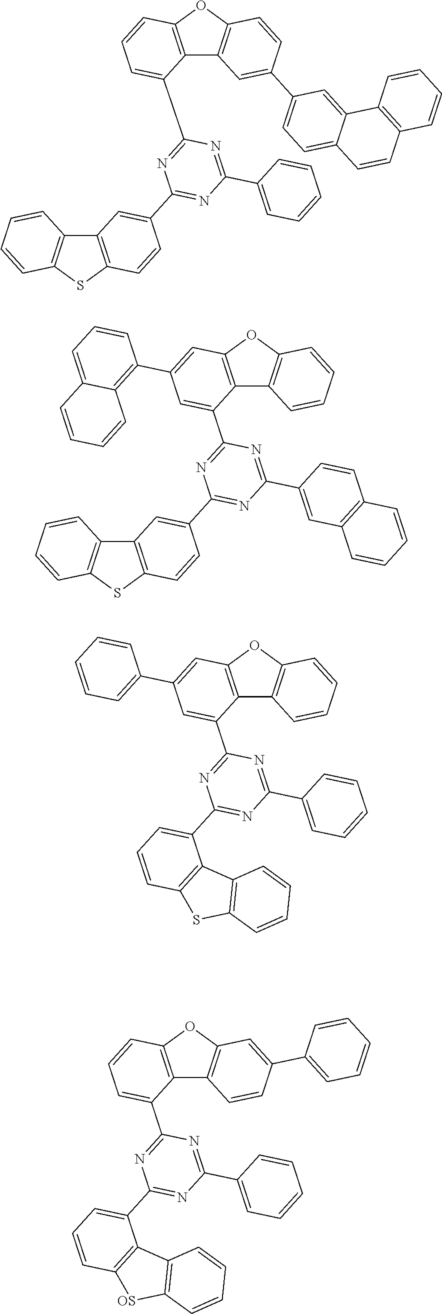

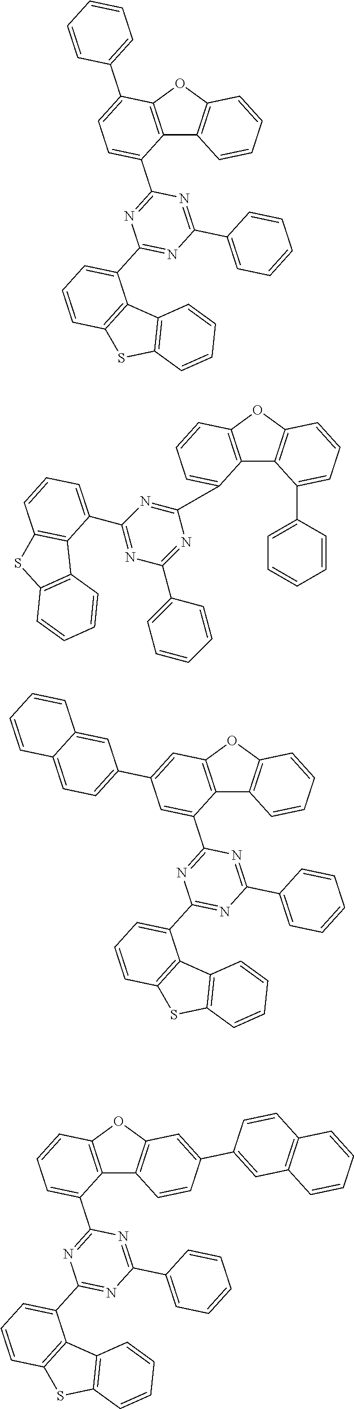 [SMILES: c1ccc(-c2nc(-c3cccc4sc5ccccc5c34)nc(-c3ccc(-c4ccccc4)c4oc5ccccc5c34)n2)cc1 ; c1ccc(-c2nc(-c3cccc4sc5ccccc5c34)nc(-c3cccc4oc5cccc(-c6ccccc6)c5c34)n2)cc1 ; c1ccc(-c2nc(-c3cc(-c4ccc5ccccc5c4)cc4oc5ccccc5c34)nc(-c3cccc4sc5ccccc5c34)n2)cc1 ; c1ccc(-c2nc(-c3cccc4oc5cc(-c6ccc7ccccc7c6)ccc5c34)nc(-c3cccc4sc5ccccc5c34)n2)cc1]

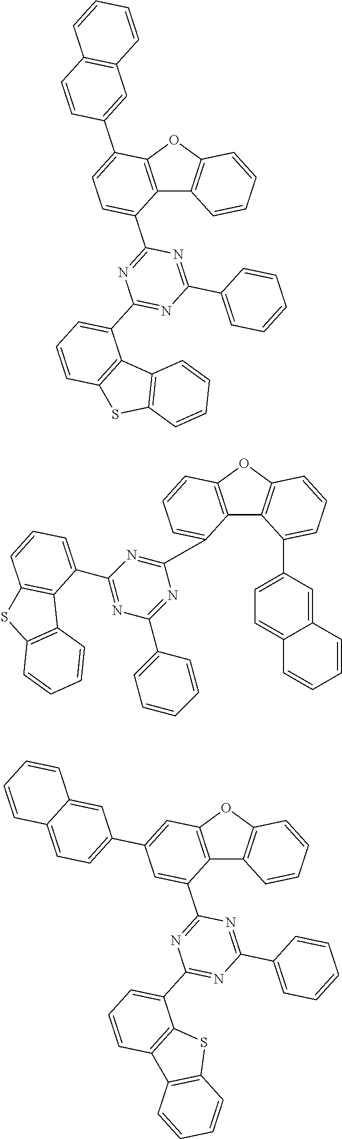 [SMILES: c1ccc(-c2nc(-c3cccc4sc5ccccc5c34)nc(-c3ccc(-c4ccc5ccccc5c4)c4oc5ccccc5c34)n2)cc1 ; c1ccc(-c2nc(-c3cccc4sc5ccccc5c34)nc(-c3cccc4oc5cccc(-c6ccc7ccccc7c6)c5c34)n2)cc1 ; c1ccc(-c2nc(-c3cccc4c3sc3ccccc34)nc(-c3cc(-c4ccc5ccccc5c4)cc4oc5ccccc5c34)n2)cc1]

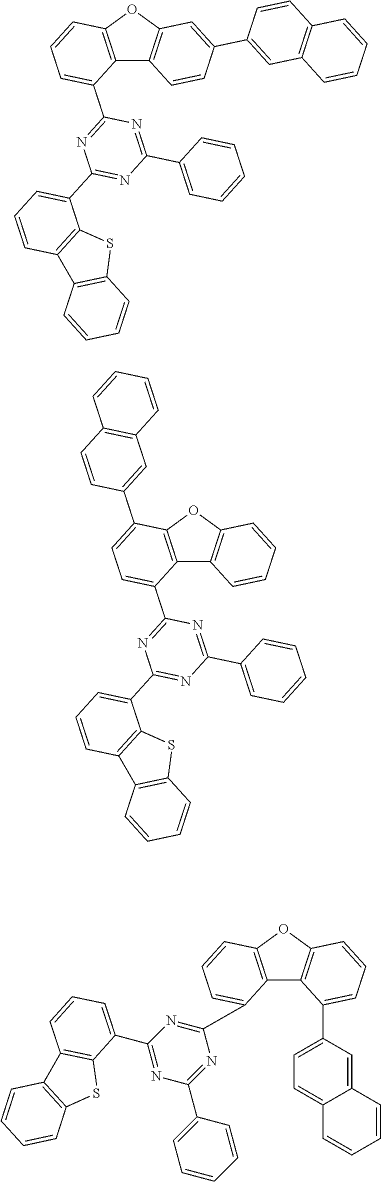

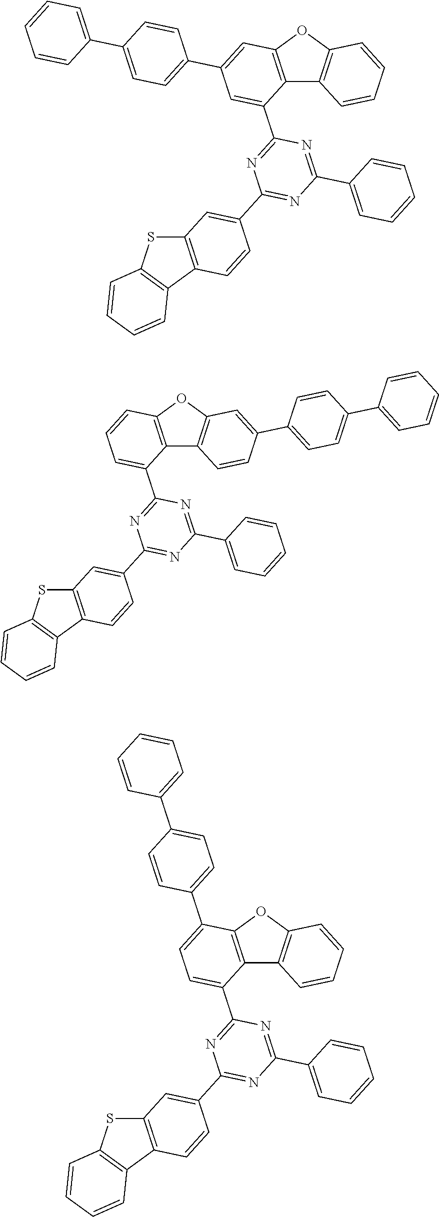 [SMILES: c1ccc(-c2ccc(-c3cc(-c4nc(-c5ccccc5)nc(-c5ccc6c(c5)sc5ccccc56)n4)c4c(c3)oc3ccccc34)cc2)cc1 ; c1ccc(-c2ccc(-c3ccc4c(c3)oc3cccc(-c5nc(-c6ccccc6)nc(-c6ccc7c(c6)sc6ccccc67)n5)c34)cc2)cc1 ; c1ccc(-c2ccc(-c3ccc(-c4nc(-c5ccccc5)nc(-c5ccc6c(c5)sc5ccccc56)n4)c4c3oc3ccccc34)cc2)cc1]

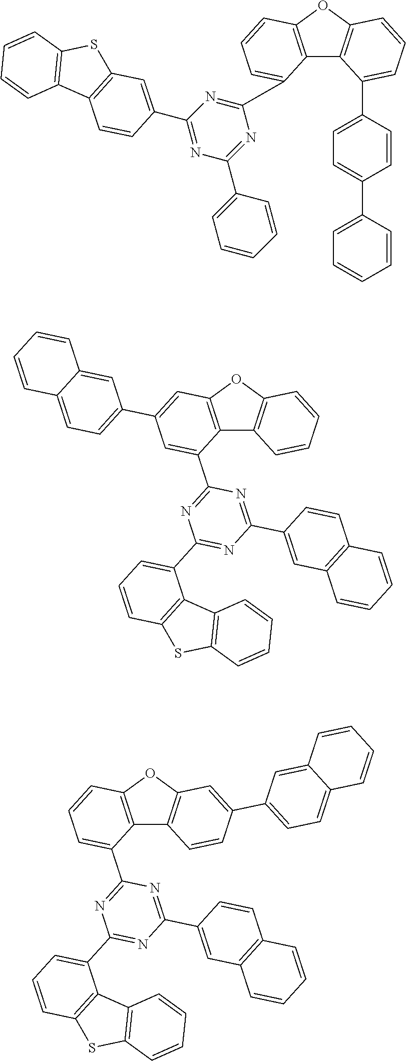 [SMILES: c1ccc(-c2ccc(-c3cccc4oc5cccc(-c6nc(-c7ccccc7)nc(-c7ccc8c(c7)sc7ccccc78)n6)c5c34)cc2)cc1 ; c1ccc2cc(-c3cc(-c4nc(-c5ccc6ccccc6c5)nc(-c5cccc6sc7ccccc7c56)n4)c4c(c3)oc3ccccc34)ccc2c1 ; c1ccc2cc(-c3ccc4c(c3)oc3cccc(-c5nc(-c6ccc7ccccc7c6)nc(-c6cccc7sc8ccccc8c67)n5)c34)ccc2c1]

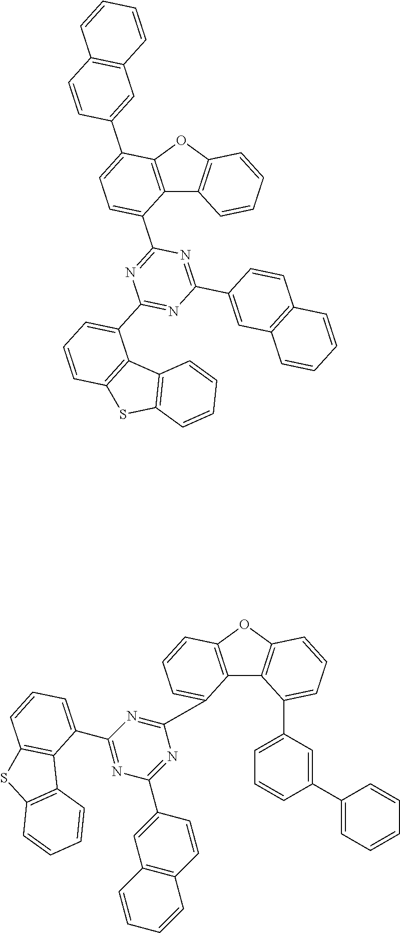 [SMILES: c1ccc2cc(-c3nc(-c4cccc5sc6ccccc6c45)nc(-c4ccc(-c5ccc6ccccc6c5)c5oc6ccccc6c45)n3)ccc2c1 ; c1ccc(-c2cccc(-c3cccc4oc5cccc(-c6nc(-c7ccc8ccccc8c7)nc(-c7cccc8sc9ccccc9c78)n6)c5c34)c2)cc1]

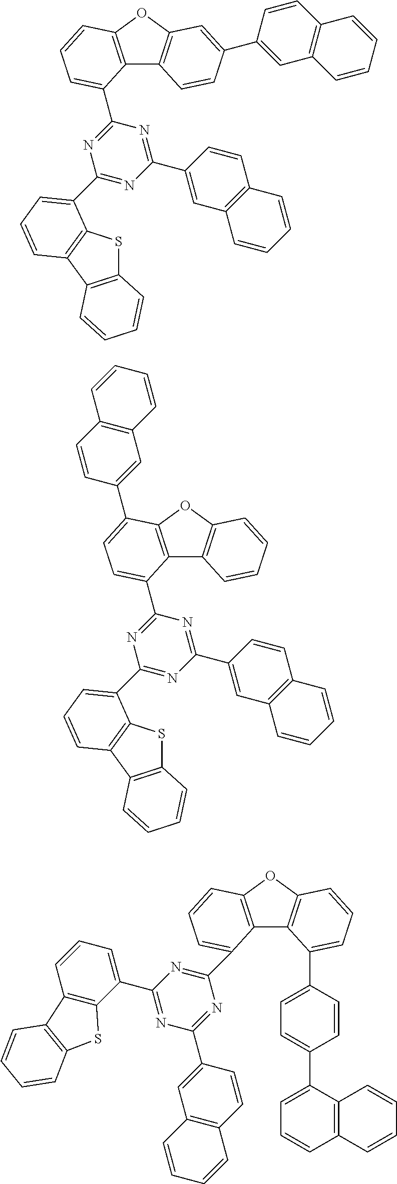

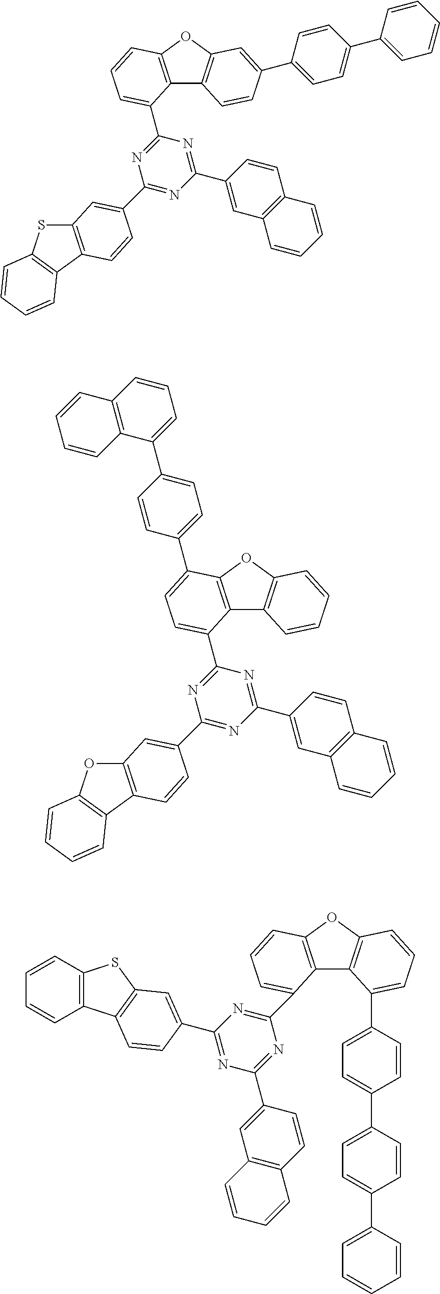 [SMILES: c1ccc(-c2ccc(-c3ccc4c(c3)oc3cccc(-c5nc(-c6ccc7ccccc7c6)nc(-c6ccc7c(c6)sc6ccccc67)n5)c34)cc2)cc1 ; c1ccc2cc(-c3nc(-c4ccc5c(c4)oc4ccccc45)nc(-c4ccc(-c5ccc(-c6cccc7ccccc67)cc5)c5oc6ccccc6c45)n3)ccc2c1 ; c1ccc(-c2ccc(-c3ccc(-c4cccc5oc6cccc(-c7nc(-c8ccc9ccccc9c8)nc(-c8ccc9c(c8)sc8ccccc89)n7)c6c45)cc3)cc2)cc1]

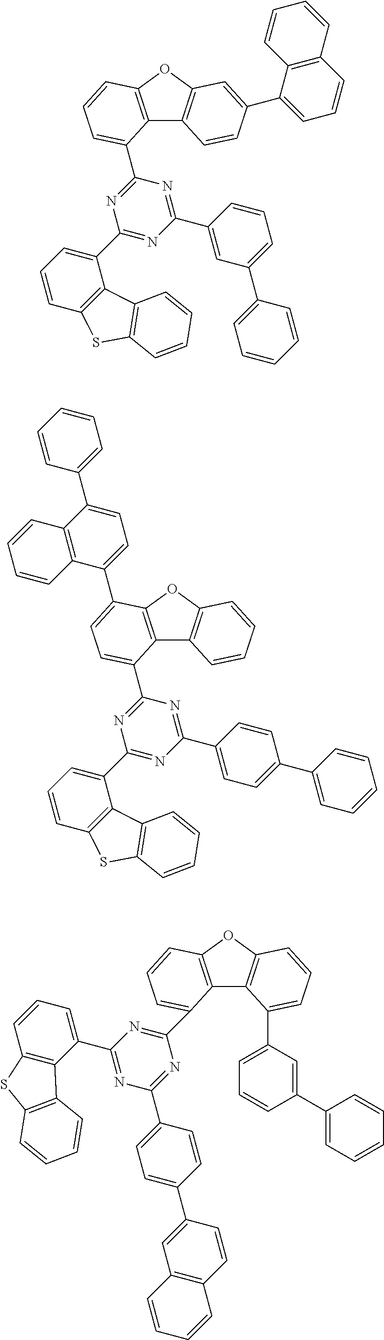 [SMILES: c1ccc(-c2cccc(-c3nc(-c4cccc5oc6cc(-c7cccc8ccccc78)ccc6c45)nc(-c4cccc5sc6ccccc6c45)n3)c2)cc1 ; c1ccc(-c2ccc(-c3nc(-c4cccc5sc6ccccc6c45)nc(-c4ccc(-c5ccc(-c6ccccc6)c6ccccc56)c5oc6ccccc6c45)n3)cc2)cc1 ; c1ccc(-c2cccc(-c3cccc4oc5cccc(-c6nc(-c7ccc(-c8ccc9ccccc9c8)cc7)nc(-c7cccc8sc9ccccc9c78)n6)c5c34)c2)cc1]

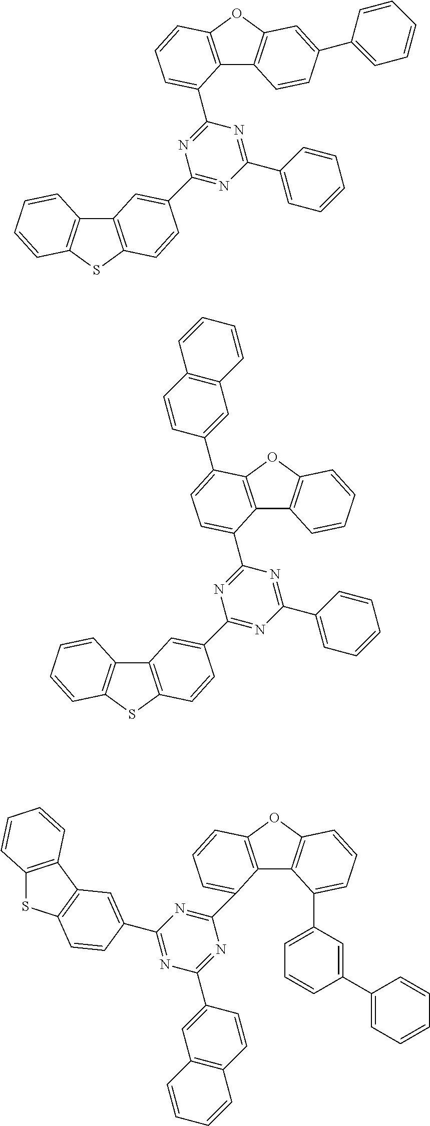 [SMILES: c1ccc(-c2ccc3c(c2)oc2cccc(-c4nc(-c5ccccc5)nc(-c5ccc6sc7ccccc7c6c5)n4)c23)cc1 ; c1ccc(-c2nc(-c3ccc4sc5ccccc5c4c3)nc(-c3ccc(-c4ccc5ccccc5c4)c4oc5ccccc5c34)n2)cc1 ; c1ccc(-c2cccc(-c3cccc4oc5cccc(-c6nc(-c7ccc8ccccc8c7)nc(-c7ccc8sc9ccccc9c8c7)n6)c5c34)c2)cc1]

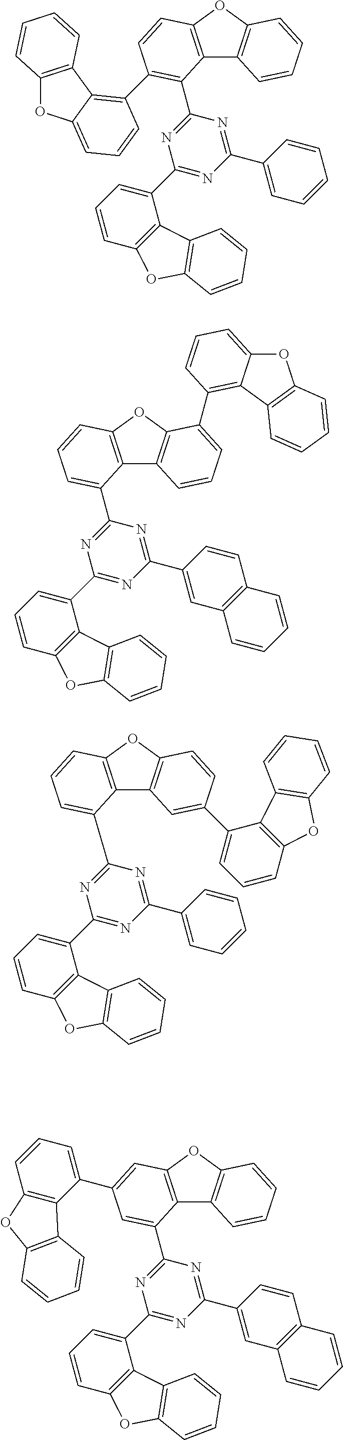

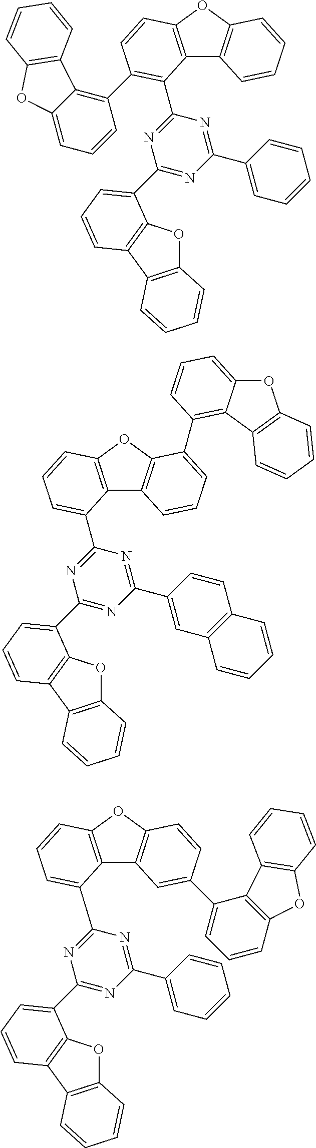

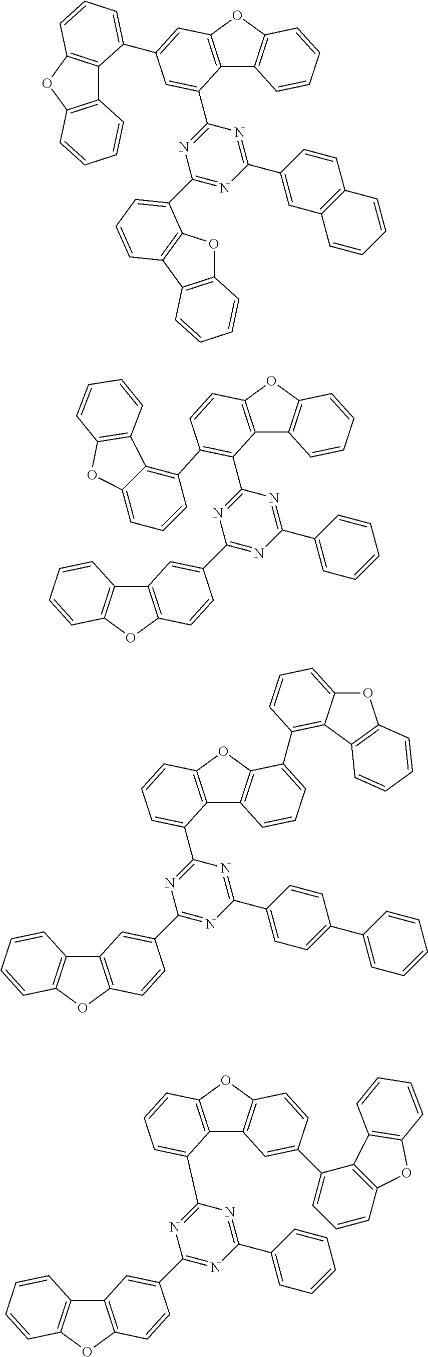 [SMILES: c1ccc2cc(-c3nc(-c4cccc5c4oc4ccccc45)nc(-c4cc(-c5cccc6oc7ccccc7c56)cc5oc6ccccc6c45)n3)ccc2c1 ; c1ccc(-c2nc(-c3ccc4oc5ccccc5c4c3)nc(-c3c(-c4cccc5oc6ccccc6c45)ccc4oc5ccccc5c34)n2)cc1 ; c1ccc(-c2ccc(-c3nc(-c4ccc5oc6ccccc6c5c4)nc(-c4cccc5oc6c(-c7cccc8oc9ccccc9c78)cccc6c45)n3)cc2)cc1 ; c1ccc(-c2nc(-c3ccc4oc5ccccc5c4c3)nc(-c3cccc4oc5ccc(-c6cccc7oc8ccccc8c67)cc5c34)n2)cc1]

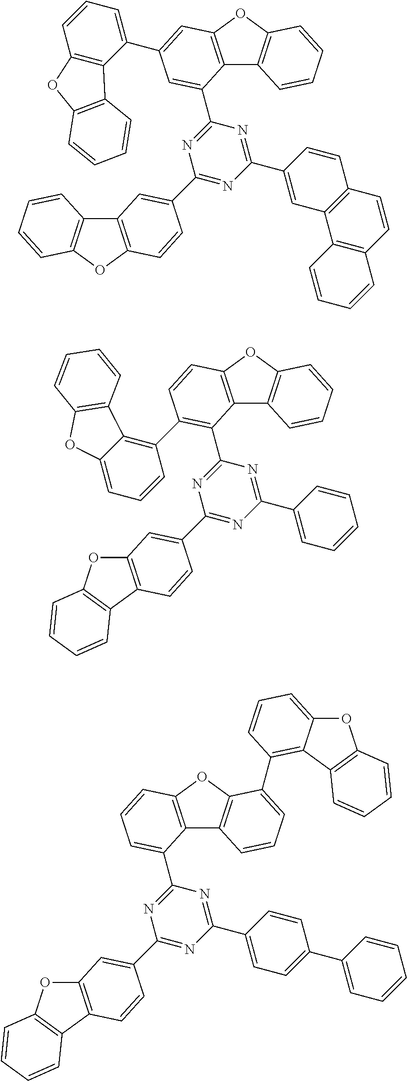 [SMILES: c1ccc2c(c1)ccc1ccc(-c3nc(-c4ccc5oc6ccccc6c5c4)nc(-c4cc(-c5cccc6oc7ccccc7c56)cc5oc6ccccc6c45)n3)cc12 ; c1ccc(-c2nc(-c3ccc4c(c3)oc3ccccc34)nc(-c3c(-c4cccc5oc6ccccc6c45)ccc4oc5ccccc5c34)n2)cc1 ; c1ccc(-c2ccc(-c3nc(-c4ccc5c(c4)oc4ccccc45)nc(-c4cccc5oc6c(-c7cccc8oc9ccccc9c78)cccc6c45)n3)cc2)cc1]

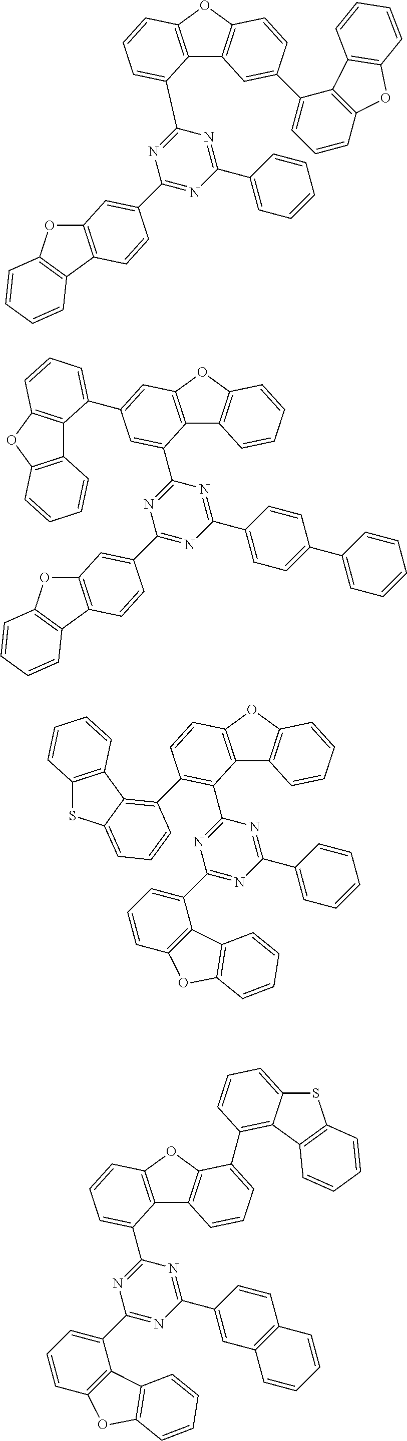

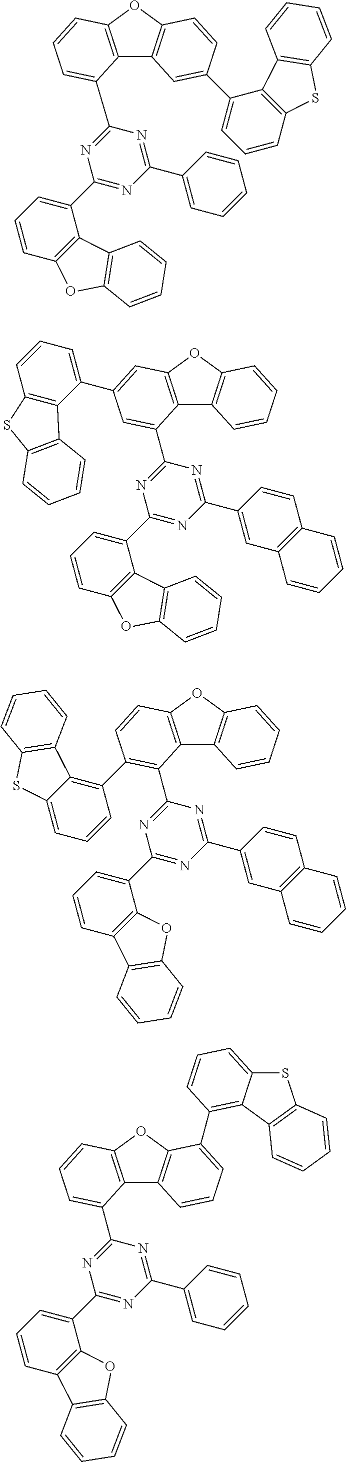

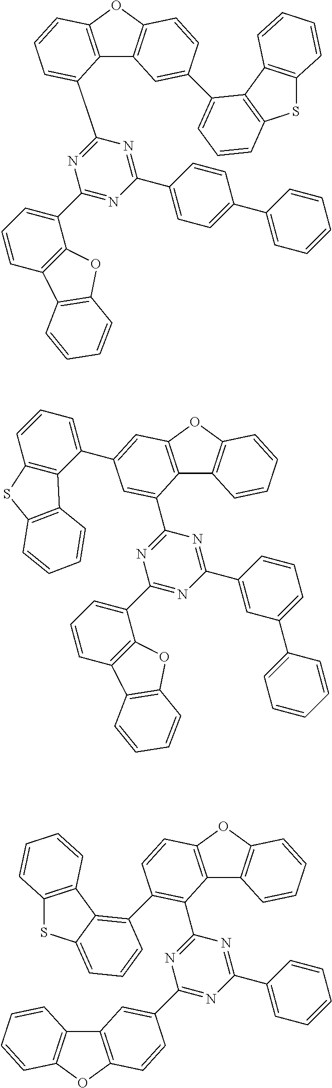 [SMILES: c1ccc(-c2ccc(-c3nc(-c4cccc5c4oc4ccccc45)nc(-c4cccc5oc6ccc(-c7cccc8sc9ccccc9c78)cc6c45)n3)cc2)cc1 ; c1ccc(-c2cccc(-c3nc(-c4cccc5c4oc4ccccc45)nc(-c4cc(-c5cccc6sc7ccccc7c56)cc5oc6ccccc6c45)n3)c2)cc1 ; c1ccc(-c2nc(-c3ccc4oc5ccccc5c4c3)nc(-c3c(-c4cccc5sc6ccccc6c45)ccc4oc5ccccc5c34)n2)cc1]

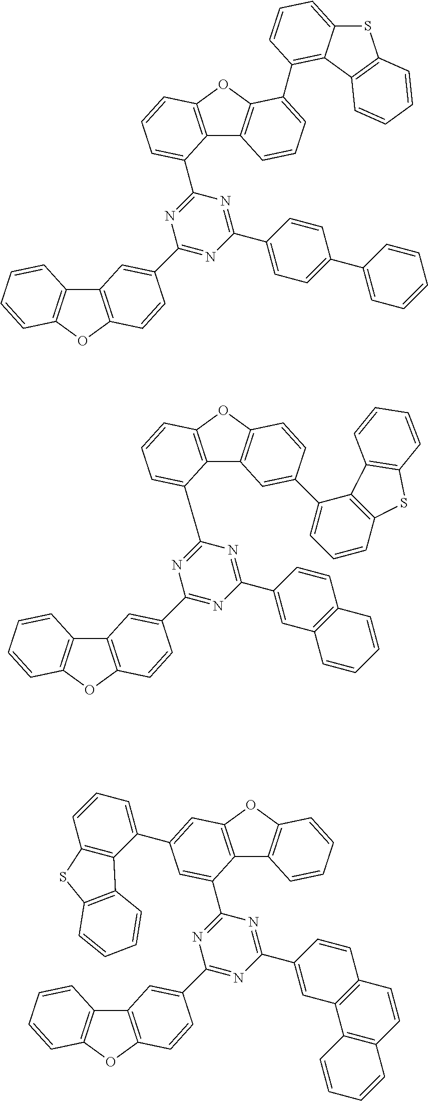 [SMILES: c1ccc(-c2ccc(-c3nc(-c4ccc5oc6ccccc6c5c4)nc(-c4cccc5oc6c(-c7cccc8sc9ccccc9c78)cccc6c45)n3)cc2)cc1 ; c1ccc2cc(-c3nc(-c4ccc5oc6ccccc6c5c4)nc(-c4cccc5oc6ccc(-c7cccc8sc9ccccc9c78)cc6c45)n3)ccc2c1 ; c1ccc2c(c1)ccc1ccc(-c3nc(-c4ccc5oc6ccccc6c5c4)nc(-c4cc(-c5cccc6sc7ccccc7c56)cc5oc6ccccc6c45)n3)cc12]

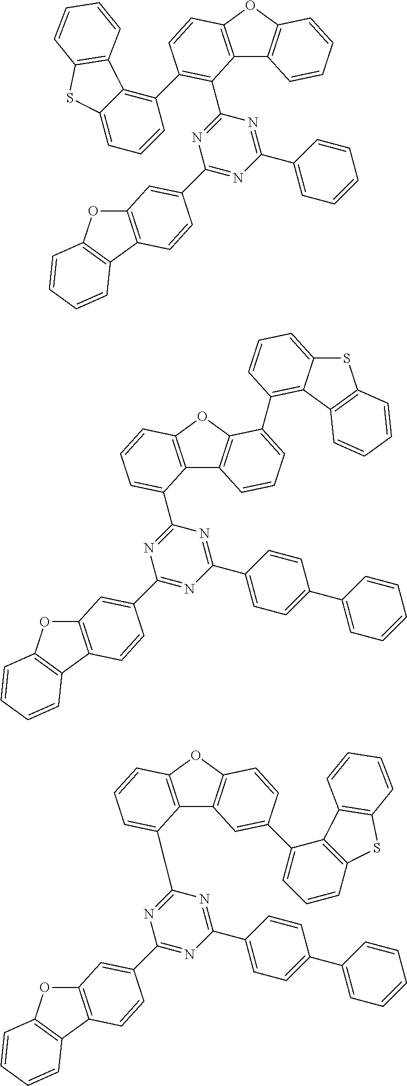 [SMILES: c1ccc(-c2nc(-c3ccc4c(c3)oc3ccccc34)nc(-c3c(-c4cccc5sc6ccccc6c45)ccc4oc5ccccc5c34)n2)cc1 ; c1ccc(-c2ccc(-c3nc(-c4ccc5c(c4)oc4ccccc45)nc(-c4cccc5oc6c(-c7cccc8sc9ccccc9c78)cccc6c45)n3)cc2)cc1 ; c1ccc(-c2ccc(-c3nc(-c4ccc5c(c4)oc4ccccc45)nc(-c4cccc5oc6ccc(-c7cccc8sc9ccccc9c78)cc6c45)n3)cc2)cc1]

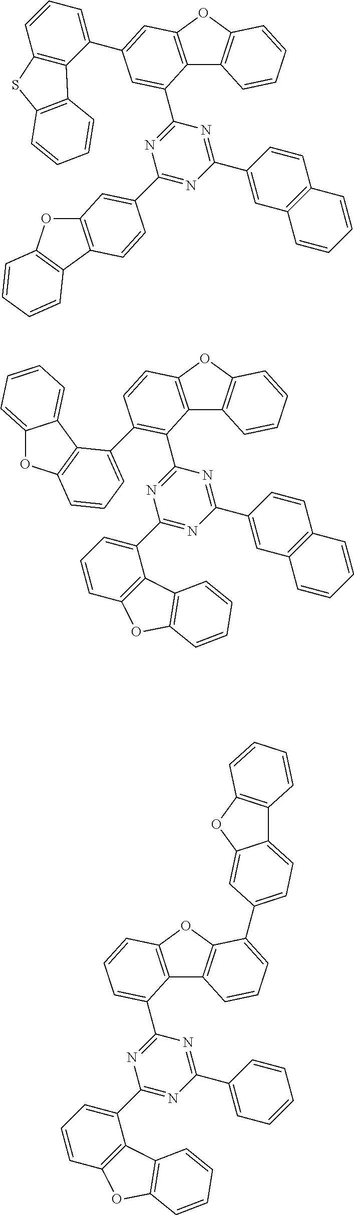 [SMILES: c1ccc2cc(-c3nc(-c4ccc5c(c4)oc4ccccc45)nc(-c4cc(-c5cccc6sc7ccccc7c56)cc5oc6ccccc6c45)n3)ccc2c1 ; c1ccc2cc(-c3nc(-c4cccc5oc6ccccc6c45)nc(-c4c(-c5cccc6oc7ccccc7c56)ccc5oc6ccccc6c45)n3)ccc2c1 ; c1ccc(-c2nc(-c3cccc4oc5ccccc5c34)nc(-c3cccc4oc5c(-c6ccc7c(c6)oc6ccccc67)cccc5c34)n2)cc1]

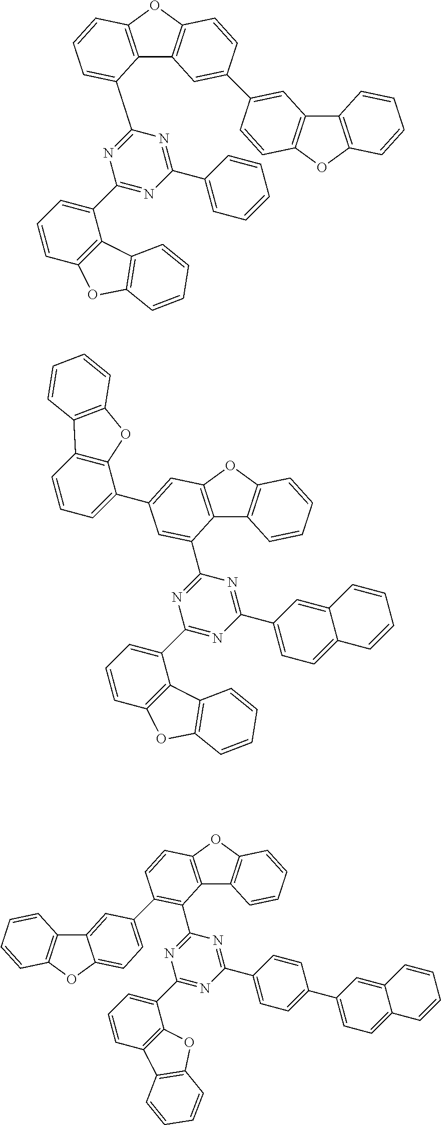 [SMILES: c1ccc(-c2nc(-c3cccc4oc5ccccc5c34)nc(-c3cccc4oc5ccc(-c6ccc7oc8ccccc8c7c6)cc5c34)n2)cc1 ; c1ccc2cc(-c3nc(-c4cccc5oc6ccccc6c45)nc(-c4cc(-c5cccc6c5oc5ccccc56)cc5oc6ccccc6c45)n3)ccc2c1 ; c1ccc2cc(-c3ccc(-c4nc(-c5cccc6c5oc5ccccc56)nc(-c5c(-c6ccc7oc8ccccc8c7c6)ccc6oc7ccccc7c56)n4)cc3)ccc2c1]

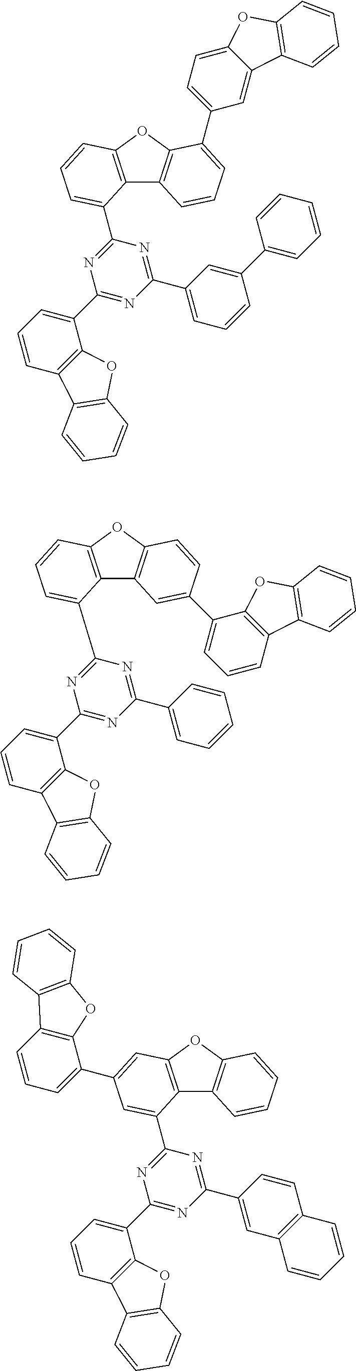 [SMILES: c1ccc(-c2cccc(-c3nc(-c4cccc5c4oc4ccccc45)nc(-c4cccc5oc6c(-c7ccc8oc9ccccc9c8c7)cccc6c45)n3)c2)cc1 ; c1ccc(-c2nc(-c3cccc4c3oc3ccccc34)nc(-c3cccc4oc5ccc(-c6cccc7c6oc6ccccc67)cc5c34)n2)cc1 ; c1ccc2cc(-c3nc(-c4cccc5c4oc4ccccc45)nc(-c4cc(-c5cccc6c5oc5ccccc56)cc5oc6ccccc6c45)n3)ccc2c1]

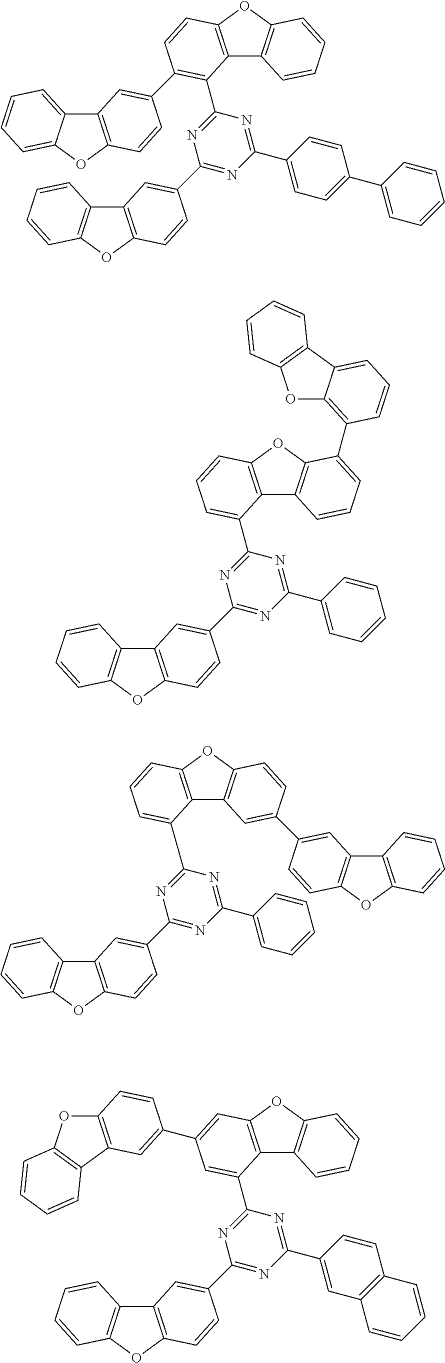 [SMILES: c1ccc(-c2ccc(-c3nc(-c4ccc5oc6ccccc6c5c4)nc(-c4c(-c5ccc6oc7ccccc7c6c5)ccc5oc6ccccc6c45)n3)cc2)cc1 ; c1ccc(-c2nc(-c3ccc4oc5ccccc5c4c3)nc(-c3cccc4oc5c(-c6cccc7c6oc6ccccc67)cccc5c34)n2)cc1 ; c1ccc(-c2nc(-c3ccc4oc5ccccc5c4c3)nc(-c3cccc4oc5ccc(-c6ccc7oc8ccccc8c7c6)cc5c34)n2)cc1 ; c1ccc2cc(-c3nc(-c4ccc5oc6ccccc6c5c4)nc(-c4cc(-c5ccc6oc7ccccc7c6c5)cc5oc6ccccc6c45)n3)ccc2c1]

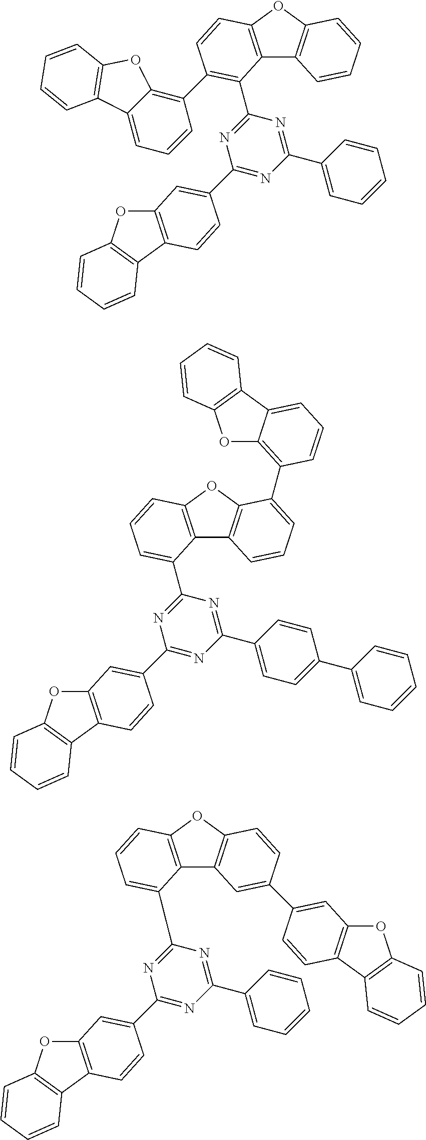 [SMILES: c1ccc(-c2nc(-c3ccc4c(c3)oc3ccccc34)nc(-c3c(-c4cccc5c4oc4ccccc45)ccc4oc5ccccc5c34)n2)cc1 ; c1ccc(-c2ccc(-c3nc(-c4ccc5c(c4)oc4ccccc45)nc(-c4cccc5oc6c(-c7cccc8c7oc7ccccc78)cccc6c45)n3)cc2)cc1 ; c1ccc(-c2nc(-c3ccc4c(c3)oc3ccccc34)nc(-c3cccc4oc5ccc(-c6ccc7c(c6)oc6ccccc67)cc5c34)n2)cc1]

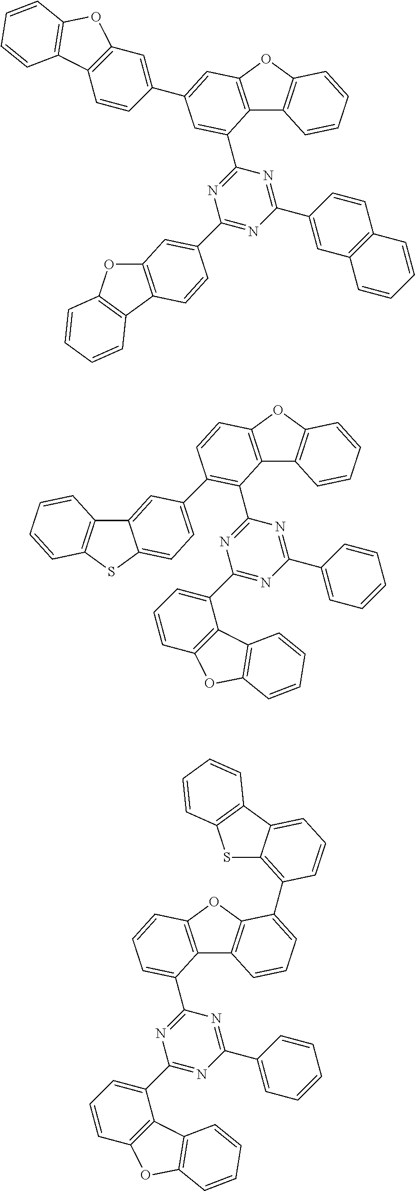 [SMILES: c1ccc2cc(-c3nc(-c4ccc5c(c4)oc4ccccc45)nc(-c4cc(-c5ccc6c(c5)oc5ccccc56)cc5oc6ccccc6c45)n3)ccc2c1 ; c1ccc(-c2nc(-c3cccc4oc5ccccc5c34)nc(-c3c(-c4ccc5sc6ccccc6c5c4)ccc4oc5ccccc5c34)n2)cc1 ; c1ccc(-c2nc(-c3cccc4oc5ccccc5c34)nc(-c3cccc4oc5c(-c6cccc7c6sc6ccccc67)cccc5c34)n2)cc1]

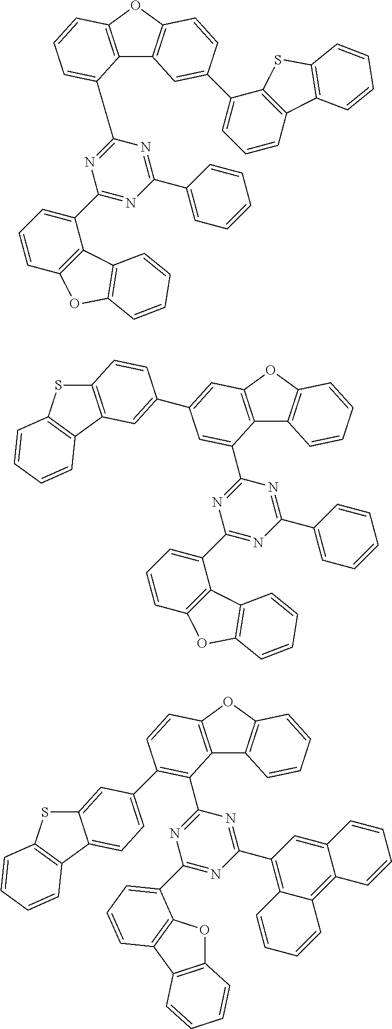

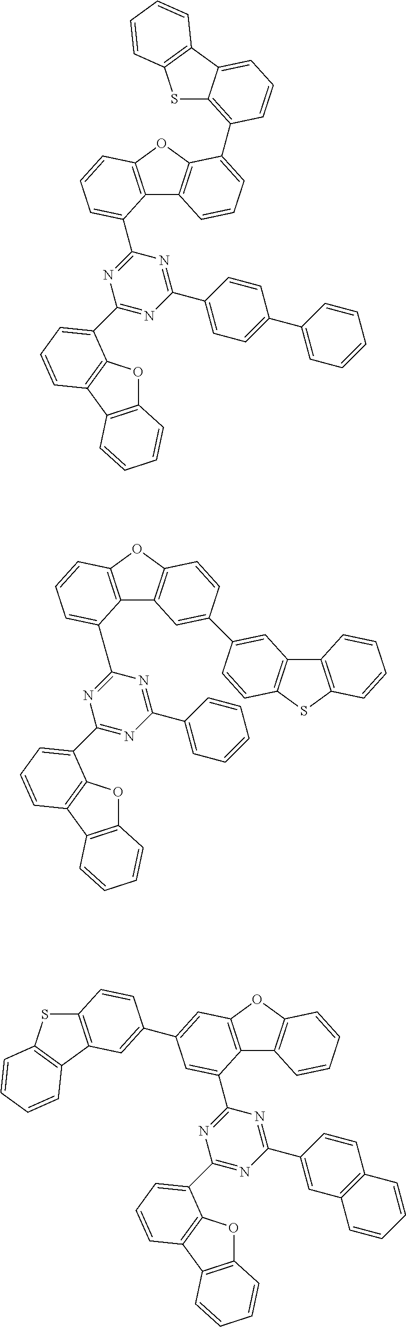 [SMILES: c1ccc(-c2ccc(-c3nc(-c4cccc5c4oc4ccccc45)nc(-c4cccc5oc6c(-c7cccc8c7sc7ccccc78)cccc6c45)n3)cc2)cc1 ; c1ccc(-c2nc(-c3cccc4c3oc3ccccc34)nc(-c3cccc4oc5ccc(-c6ccc7sc8ccccc8c7c6)cc5c34)n2)cc1 ; c1ccc2cc(-c3nc(-c4cccc5c4oc4ccccc45)nc(-c4cc(-c5ccc6sc7ccccc7c6c5)cc5oc6ccccc6c45)n3)ccc2c1]

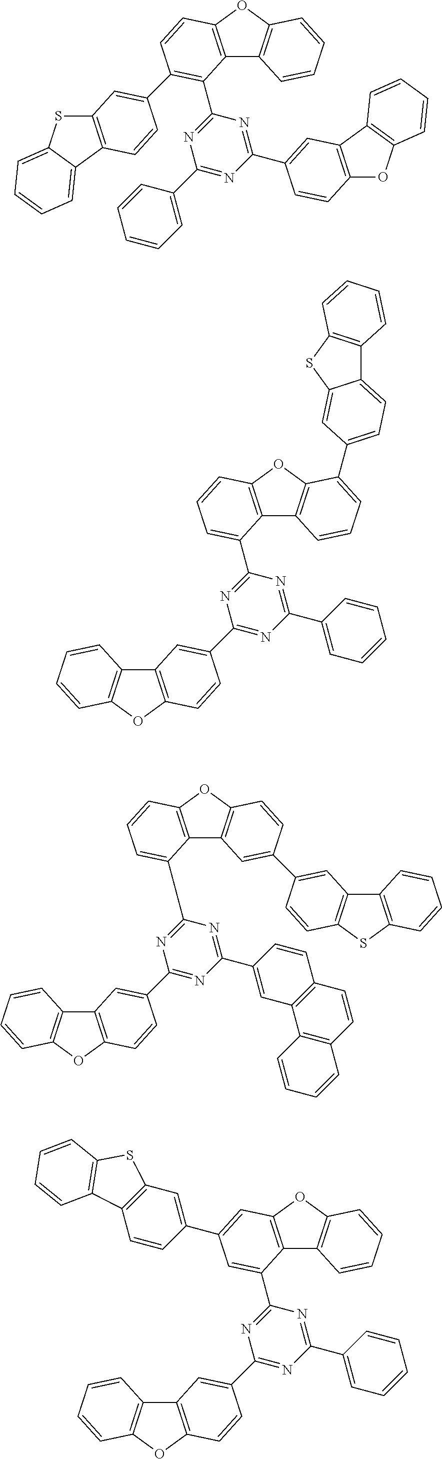 [SMILES: c1ccc(-c2nc(-c3ccc4oc5ccccc5c4c3)nc(-c3c(-c4ccc5c(c4)sc4ccccc45)ccc4oc5ccccc5c34)n2)cc1 ; c1ccc(-c2nc(-c3ccc4oc5ccccc5c4c3)nc(-c3cccc4oc5c(-c6ccc7c(c6)sc6ccccc67)cccc5c34)n2)cc1 ; c1ccc2c(c1)ccc1ccc(-c3nc(-c4ccc5oc6ccccc6c5c4)nc(-c4cccc5oc6ccc(-c7ccc8sc9ccccc9c8c7)cc6c45)n3)cc12 ; c1ccc(-c2nc(-c3ccc4oc5ccccc5c4c3)nc(-c3cc(-c4ccc5c(c4)sc4ccccc45)cc4oc5ccccc5c34)n2)cc1]

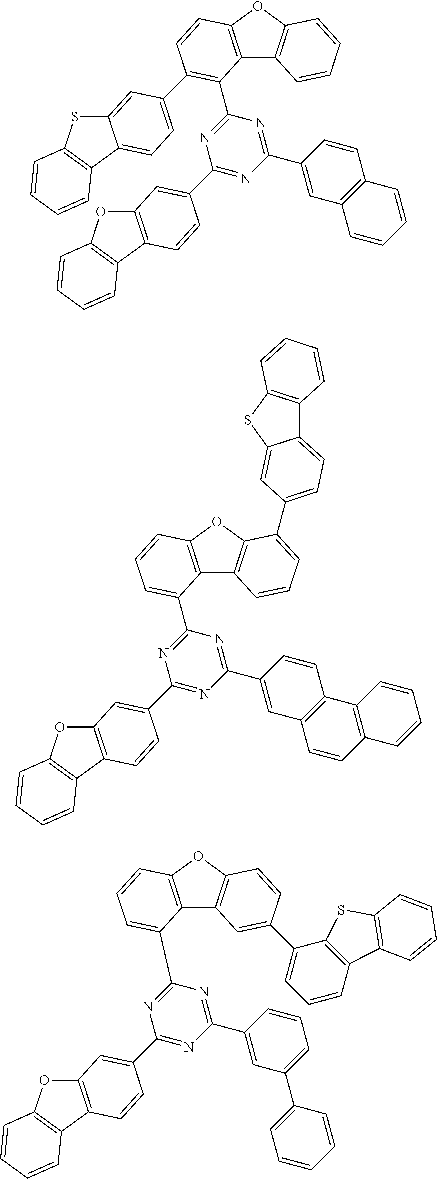 [SMILES: c1ccc2cc(-c3nc(-c4ccc5c(c4)oc4ccccc45)nc(-c4c(-c5ccc6c(c5)sc5ccccc56)ccc5oc6ccccc6c45)n3)ccc2c1 ; c1ccc2c(c1)ccc1cc(-c3nc(-c4ccc5c(c4)oc4ccccc45)nc(-c4cccc5oc6c(-c7ccc8c(c7)sc7ccccc78)cccc6c45)n3)ccc12 ; c1ccc(-c2cccc(-c3nc(-c4ccc5c(c4)oc4ccccc45)nc(-c4cccc5oc6ccc(-c7cccc8c7sc7ccccc78)cc6c45)n3)c2)cc1]

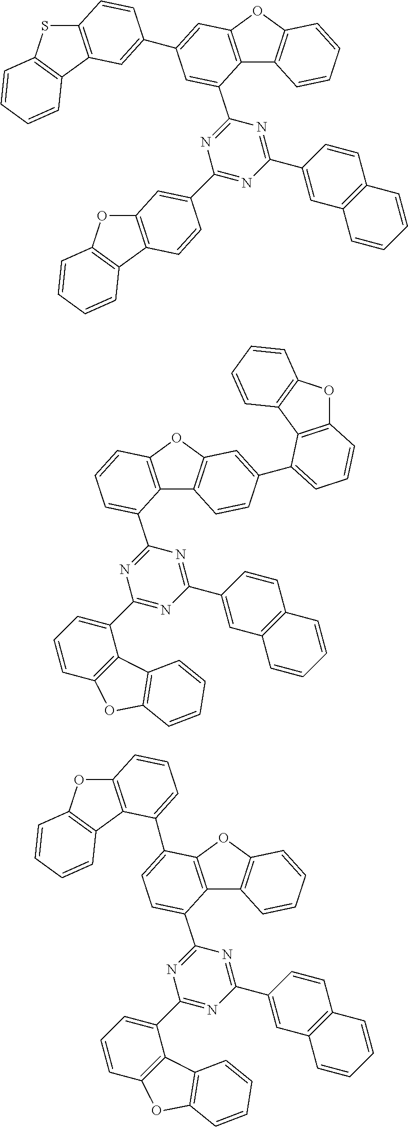

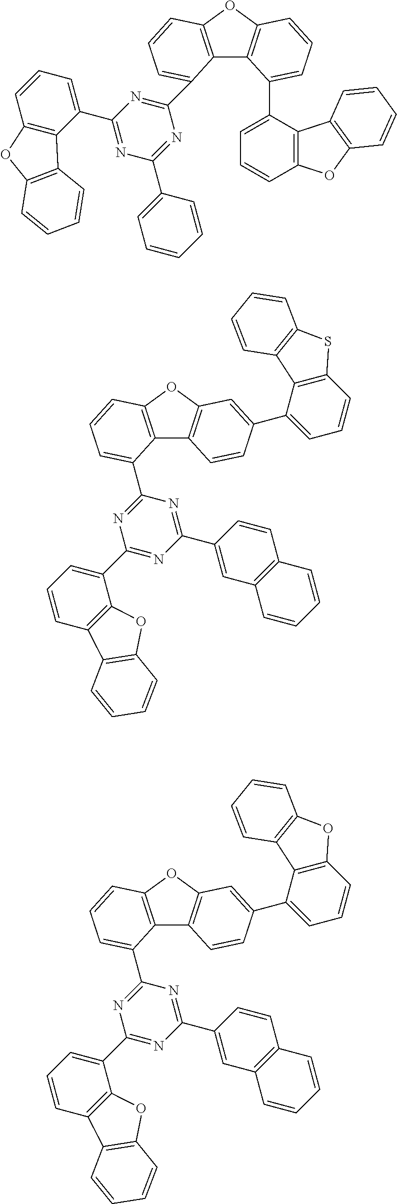 [SMILES: c1ccc(-c2nc(-c3cccc4oc5ccccc5c34)nc(-c3cccc4oc5cccc(-c6cccc7oc8ccccc8c67)c5c34)n2)cc1 ; c1ccc2cc(-c3nc(-c4cccc5c4oc4ccccc45)nc(-c4cccc5oc6cc(-c7cccc8sc9ccccc9c78)ccc6c45)n3)ccc2c1 ; c1ccc2cc(-c3nc(-c4cccc5c4oc4ccccc45)nc(-c4cccc5oc6cc(-c7cccc8oc9ccccc9c78)ccc6c45)n3)ccc2c1]

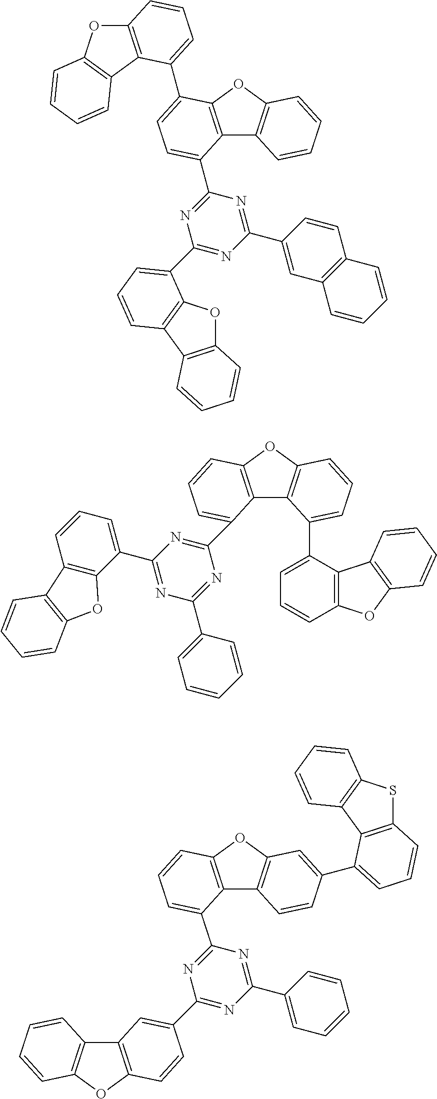 [SMILES: c1ccc2cc(-c3nc(-c4cccc5c4oc4ccccc45)nc(-c4ccc(-c5cccc6oc7ccccc7c56)c5oc6ccccc6c45)n3)ccc2c1 ; c1ccc(-c2nc(-c3cccc4c3oc3ccccc34)nc(-c3cccc4oc5cccc(-c6cccc7oc8ccccc8c67)c5c34)n2)cc1 ; c1ccc(-c2nc(-c3ccc4oc5ccccc5c4c3)nc(-c3cccc4oc5cc(-c6cccc7sc8ccccc8c67)ccc5c34)n2)cc1]

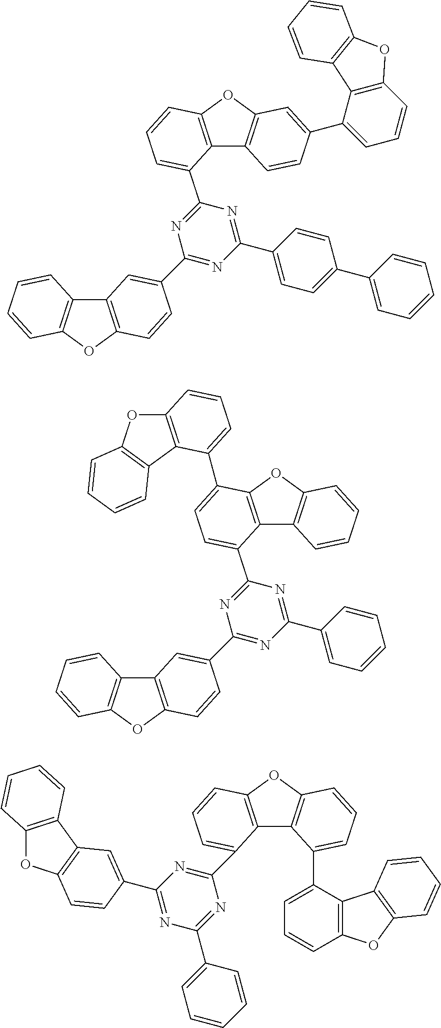 [SMILES: c1ccc(-c2ccc(-c3nc(-c4ccc5oc6ccccc6c5c4)nc(-c4cccc5oc6cc(-c7cccc8oc9ccccc9c78)ccc6c45)n3)cc2)cc1 ; c1ccc(-c2nc(-c3ccc4oc5ccccc5c4c3)nc(-c3ccc(-c4cccc5oc6ccccc6c45)c4oc5ccccc5c34)n2)cc1 ; c1ccc(-c2nc(-c3ccc4oc5ccccc5c4c3)nc(-c3cccc4oc5cccc(-c6cccc7oc8ccccc8c67)c5c34)n2)cc1]

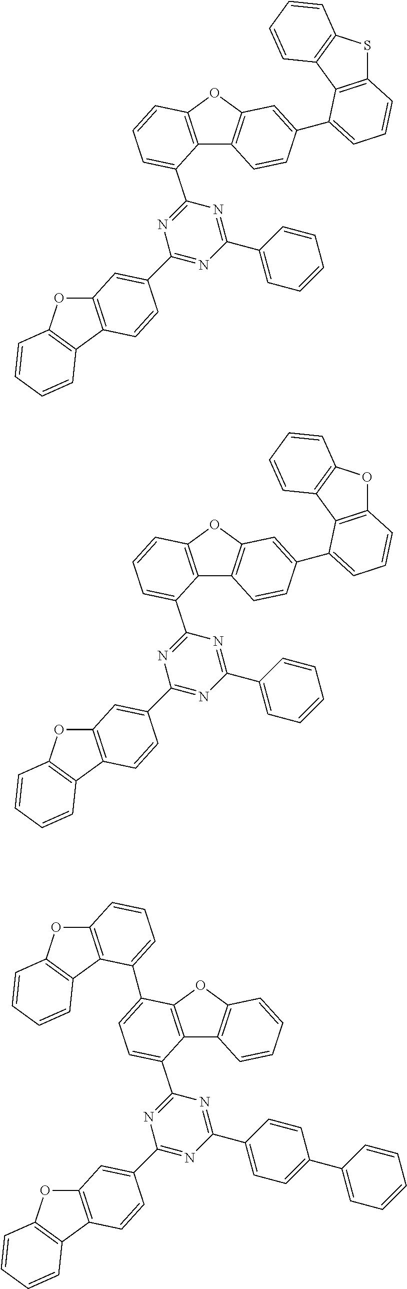 [SMILES: c1ccc(-c2nc(-c3ccc4c(c3)oc3ccccc34)nc(-c3cccc4oc5cc(-c6cccc7sc8ccccc8c67)ccc5c34)n2)cc1 ; c1ccc(-c2nc(-c3ccc4c(c3)oc3ccccc34)nc(-c3cccc4oc5cc(-c6cccc7oc8ccccc8c67)ccc5c34)n2)cc1 ; c1ccc(-c2ccc(-c3nc(-c4ccc5c(c4)oc4ccccc45)nc(-c4ccc(-c5cccc6oc7ccccc7c56)c5oc6ccccc6c45)n3)cc2)cc1]

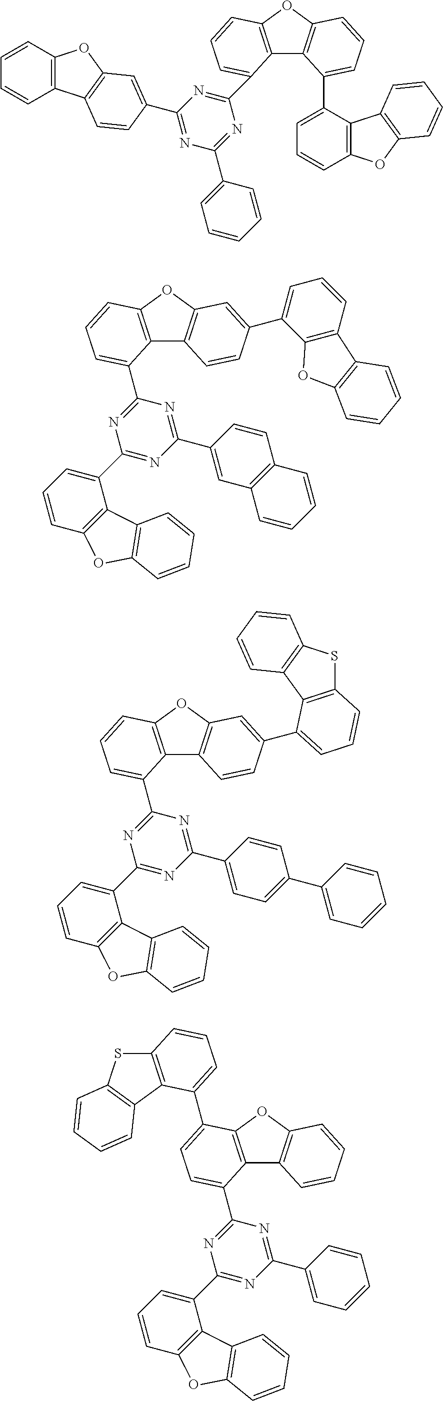

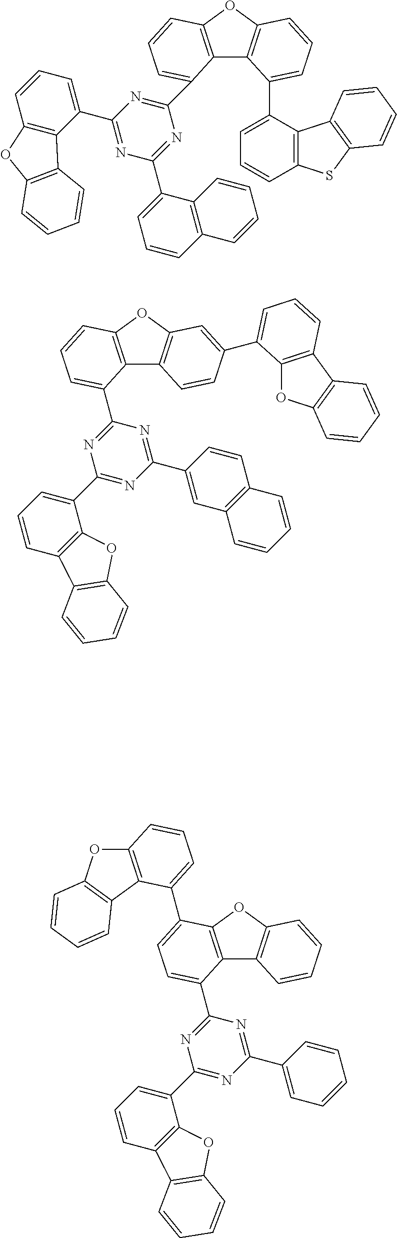 [SMILES: c1ccc2c(-c3nc(-c4cccc5oc6ccccc6c45)nc(-c4cccc5oc6cccc(-c7cccc8sc9ccccc9c78)c6c45)n3)cccc2c1 ; c1ccc2cc(-c3nc(-c4cccc5c4oc4ccccc45)nc(-c4cccc5oc6cc(-c7cccc8c7oc7ccccc78)ccc6c45)n3)ccc2c1 ; c1ccc(-c2nc(-c3cccc4c3oc3ccccc34)nc(-c3ccc(-c4cccc5oc6ccccc6c45)c4oc5ccccc5c34)n2)cc1]

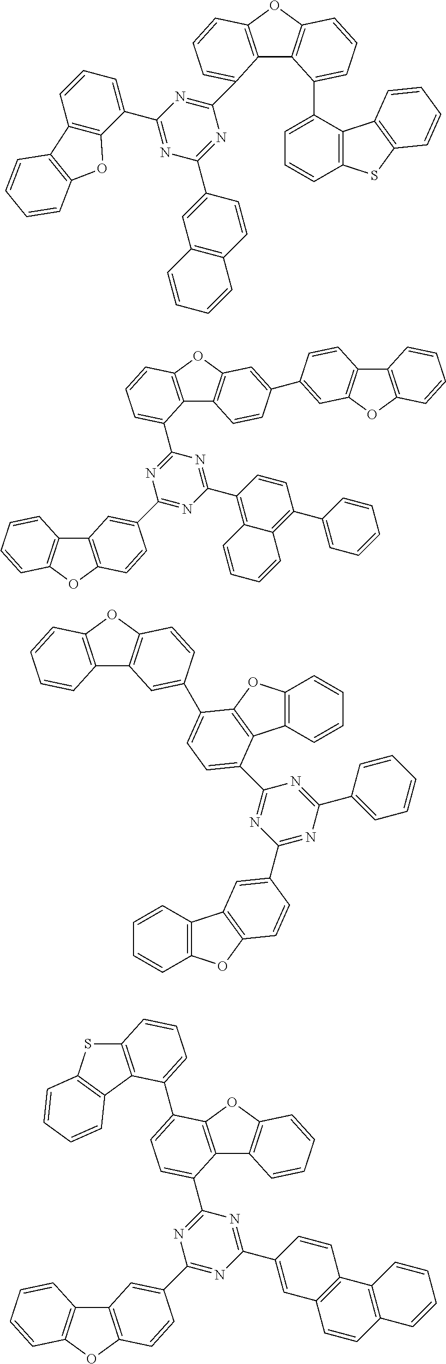

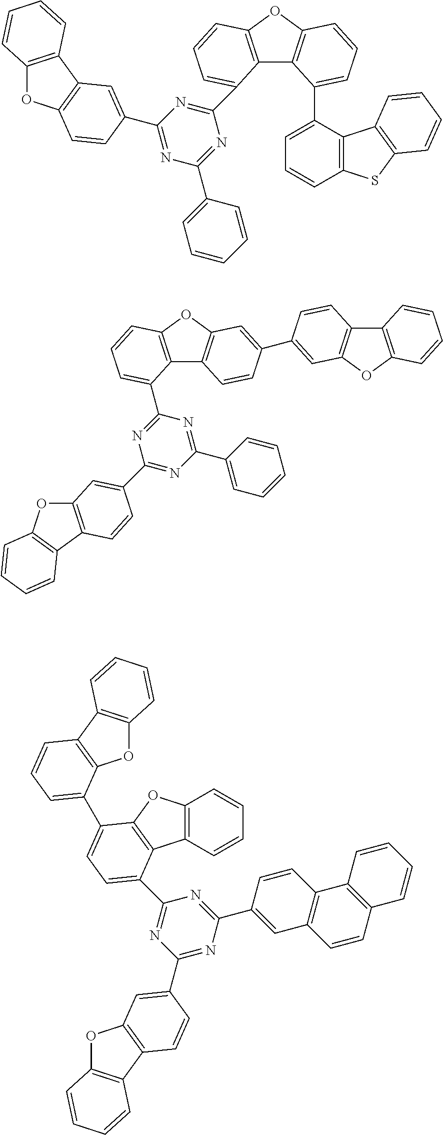 [SMILES: c1ccc(-c2nc(-c3ccc4oc5ccccc5c4c3)nc(-c3cccc4oc5cccc(-c6cccc7sc8ccccc8c67)c5c34)n2)cc1 ; c1ccc(-c2nc(-c3ccc4c(c3)oc3ccccc34)nc(-c3cccc4oc5cc(-c6ccc7c(c6)oc6ccccc67)ccc5c34)n2)cc1 ; c1ccc2c(c1)ccc1cc(-c3nc(-c4ccc5c(c4)oc4ccccc45)nc(-c4ccc(-c5cccc6c5oc5ccccc56)c5oc6ccccc6c45)n3)ccc12]

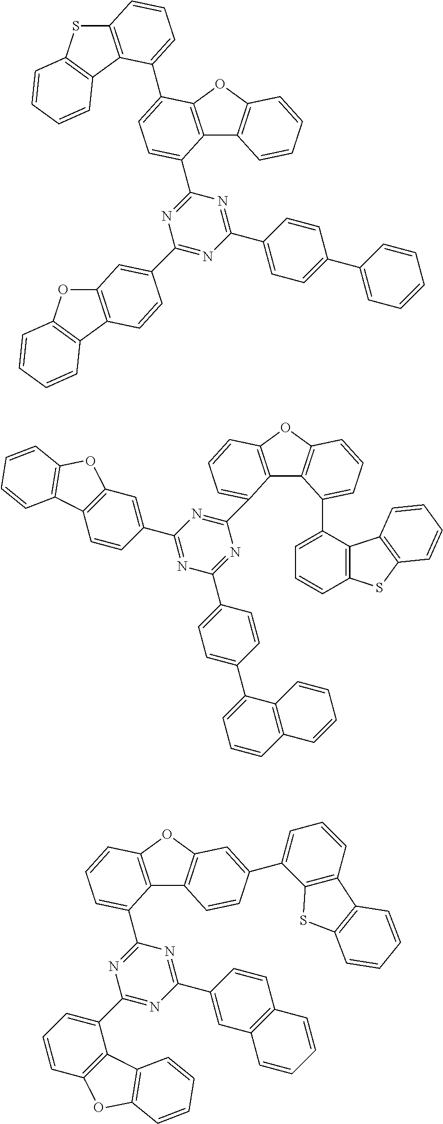 [SMILES: c1ccc(-c2ccc(-c3nc(-c4ccc5c(c4)oc4ccccc45)nc(-c4ccc(-c5cccc6sc7ccccc7c56)c5oc6ccccc6c45)n3)cc2)cc1 ; c1ccc2c(-c3ccc(-c4nc(-c5ccc6c(c5)oc5ccccc56)nc(-c5cccc6oc7cccc(-c8cccc9sc%10ccccc%10c89)c7c56)n4)cc3)cccc2c1 ; c1ccc2cc(-c3nc(-c4cccc5oc6ccccc6c45)nc(-c4cccc5oc6cc(-c7cccc8c7sc7ccccc78)ccc6c45)n3)ccc2c1]

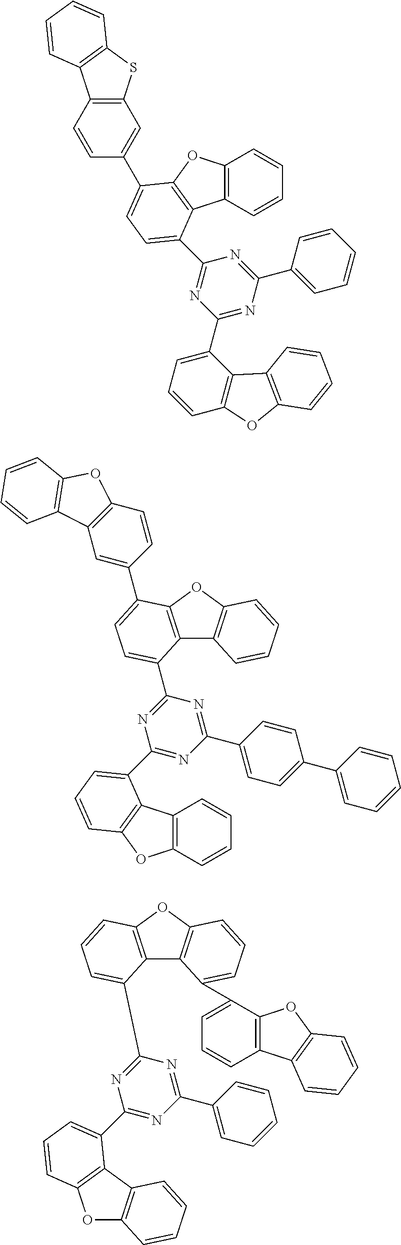 [SMILES: c1ccc(-c2nc(-c3cccc4oc5ccccc5c34)nc(-c3ccc(-c4ccc5c(c4)sc4ccccc45)c4oc5ccccc5c34)n2)cc1 ; c1ccc(-c2ccc(-c3nc(-c4cccc5oc6ccccc6c45)nc(-c4ccc(-c5ccc6oc7ccccc7c6c5)c5oc6ccccc6c45)n3)cc2)cc1 ; c1ccc(-c2nc(-c3cccc4oc5ccccc5c34)nc(-c3cccc4oc5cccc(-c6cccc7c6oc6ccccc67)c5c34)n2)cc1]

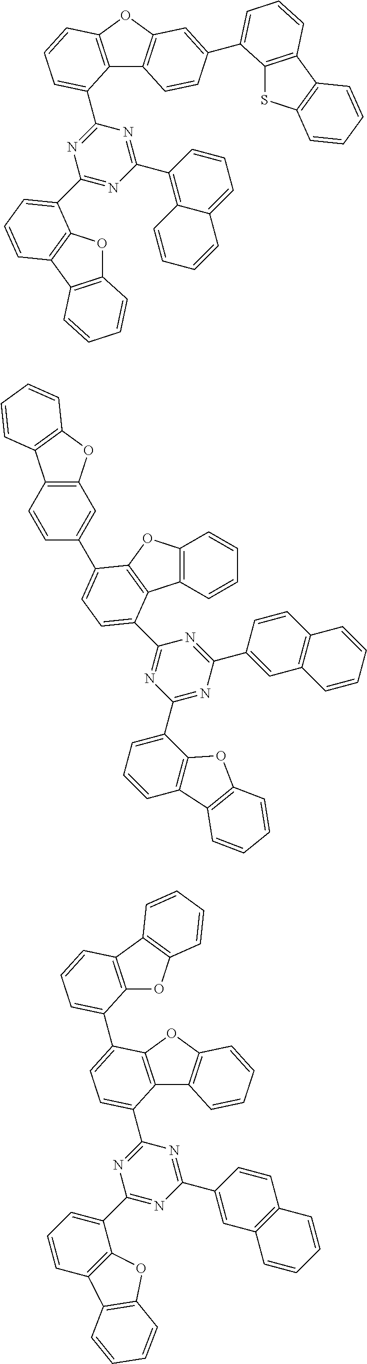 [SMILES: c1ccc2c(-c3nc(-c4cccc5c4oc4ccccc45)nc(-c4cccc5oc6cc(-c7cccc8c7sc7ccccc78)ccc6c45)n3)cccc2c1 ; c1ccc2cc(-c3nc(-c4cccc5c4oc4ccccc45)nc(-c4ccc(-c5ccc6c(c5)oc5ccccc56)c5oc6ccccc6c45)n3)ccc2c1 ; c1ccc2cc(-c3nc(-c4cccc5c4oc4ccccc45)nc(-c4ccc(-c5cccc6c5oc5ccccc56)c5oc6ccccc6c45)n3)ccc2c1]

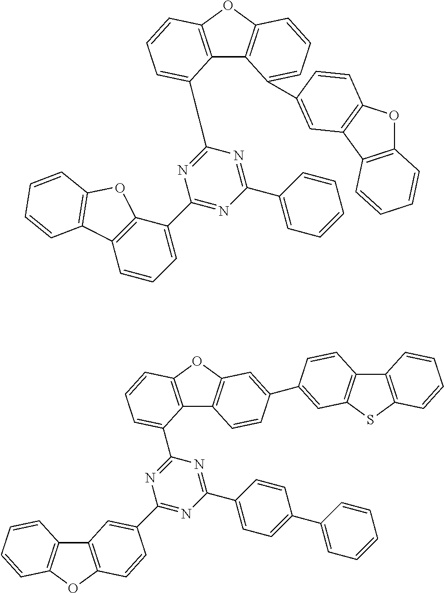 [SMILES: c1ccc(-c2nc(-c3cccc4c3oc3ccccc34)nc(-c3cccc4oc5cccc(-c6ccc7oc8ccccc8c7c6)c5c34)n2)cc1 ; c1ccc(-c2ccc(-c3nc(-c4ccc5oc6ccccc6c5c4)nc(-c4cccc5oc6cc(-c7ccc8c(c7)sc7ccccc78)ccc6c45)n3)cc2)cc1]

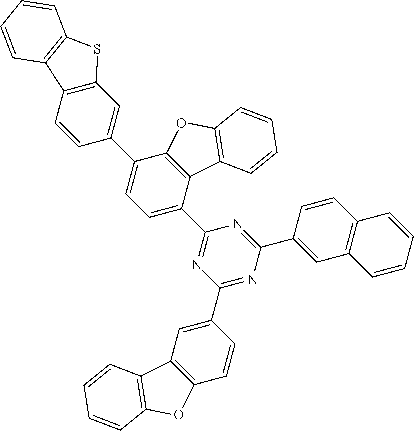 [SMILES: c1ccc2cc(-c3nc(-c4ccc5oc6ccccc6c5c4)nc(-c4ccc(-c5ccc6c(c5)sc5ccccc56)c5oc6ccccc6c45)n3)ccc2c1]

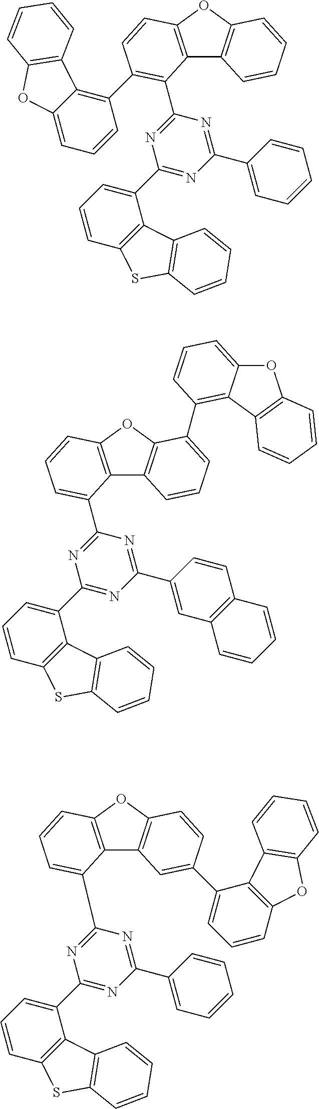 [SMILES: c1ccc(-c2nc(-c3c(-c4cccc5oc6ccccc6c45)ccc4oc5ccccc5c34)nc(-c3cccc4sc5ccccc5c34)n2)cc1 ; c1ccc2cc(-c3nc(-c4cccc5oc6c(-c7cccc8oc9ccccc9c78)cccc6c45)nc(-c4cccc5sc6ccccc6c45)n3)ccc2c1 ; c1ccc(-c2nc(-c3cccc4oc5ccc(-c6cccc7oc8ccccc8c67)cc5c34)nc(-c3cccc4sc5ccccc5c34)n2)cc1]

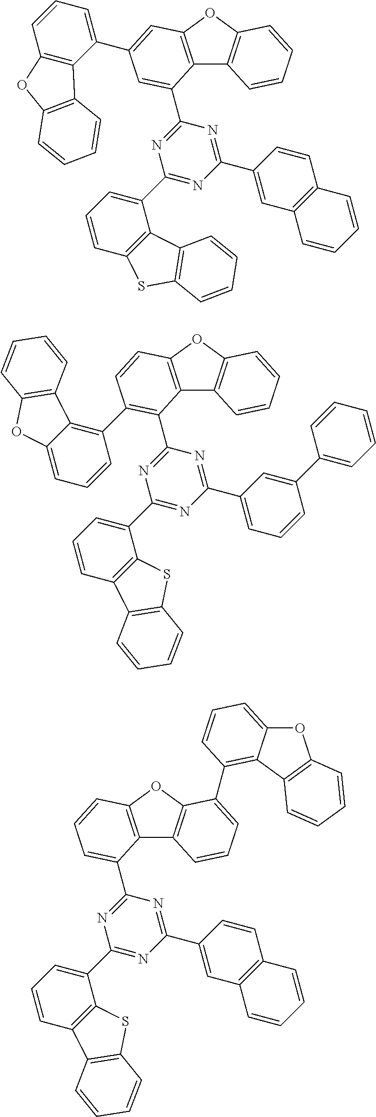

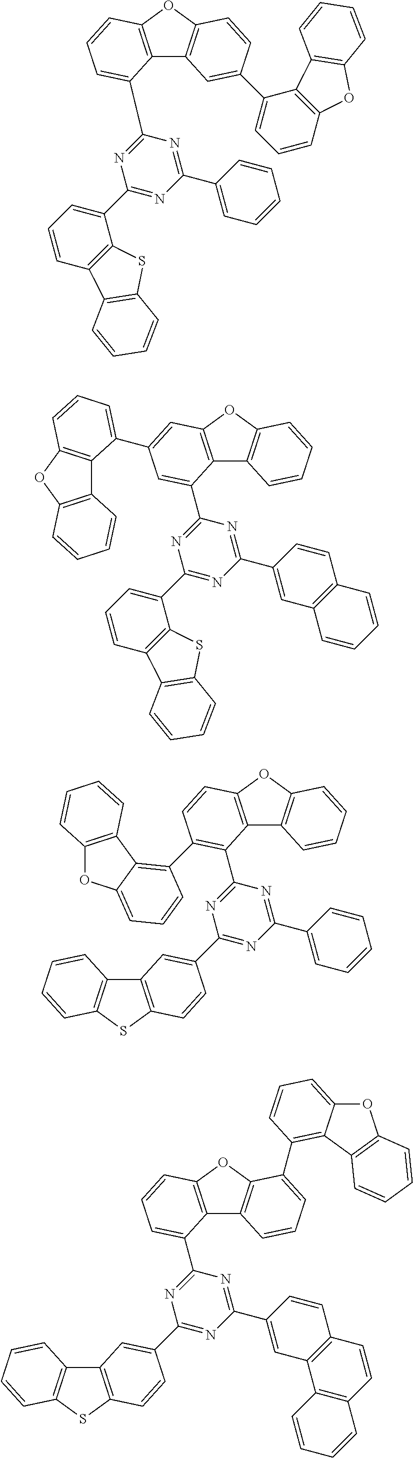 [SMILES: c1ccc(-c2nc(-c3cccc4c3sc3ccccc34)nc(-c3cccc4oc5ccc(-c6cccc7oc8ccccc8c67)cc5c34)n2)cc1 ; c1ccc2cc(-c3nc(-c4cccc5c4sc4ccccc45)nc(-c4cc(-c5cccc6oc7ccccc7c56)cc5oc6ccccc6c45)n3)ccc2c1 ; c1ccc(-c2nc(-c3ccc4sc5ccccc5c4c3)nc(-c3c(-c4cccc5oc6ccccc6c45)ccc4oc5ccccc5c34)n2)cc1 ; c1ccc2c(c1)ccc1ccc(-c3nc(-c4ccc5sc6ccccc6c5c4)nc(-c4cccc5oc6c(-c7cccc8oc9ccccc9c78)cccc6c45)n3)cc12]

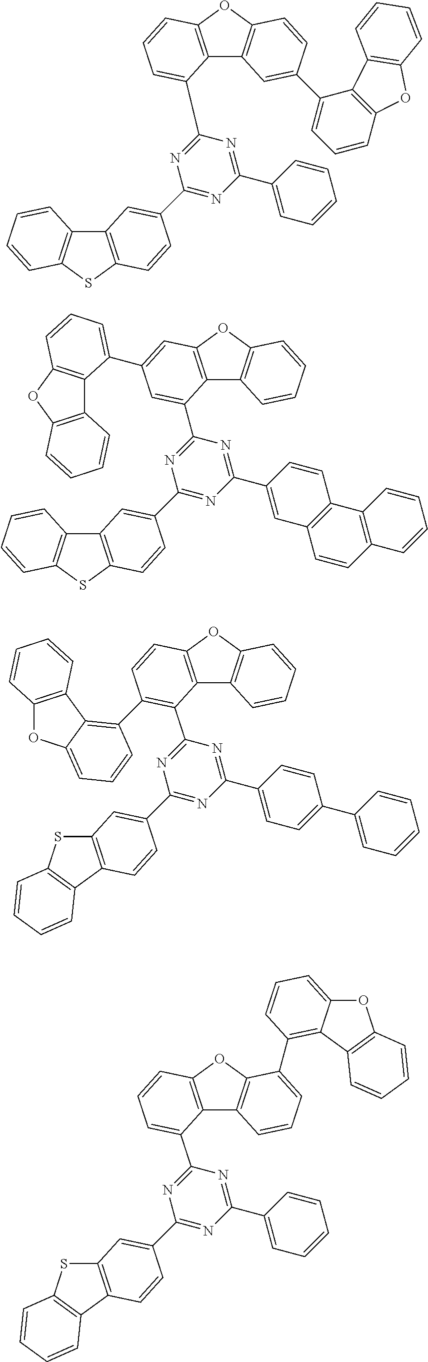

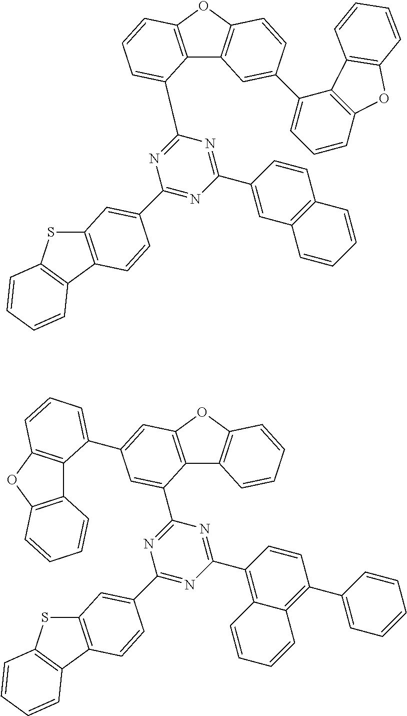 [SMILES: c1ccc2cc(-c3nc(-c4ccc5c(c4)sc4ccccc45)nc(-c4cccc5oc6ccc(-c7cccc8oc9ccccc9c78)cc6c45)n3)ccc2c1 ; c1ccc(-c2ccc(-c3nc(-c4ccc5c(c4)sc4ccccc45)nc(-c4cc(-c5cccc6oc7ccccc7c56)cc5oc6ccccc6c45)n3)c3ccccc23)cc1]

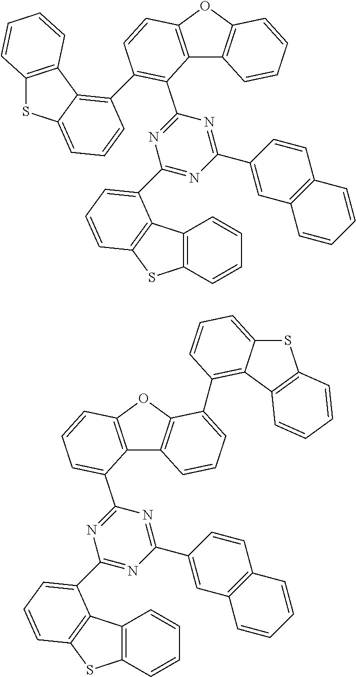

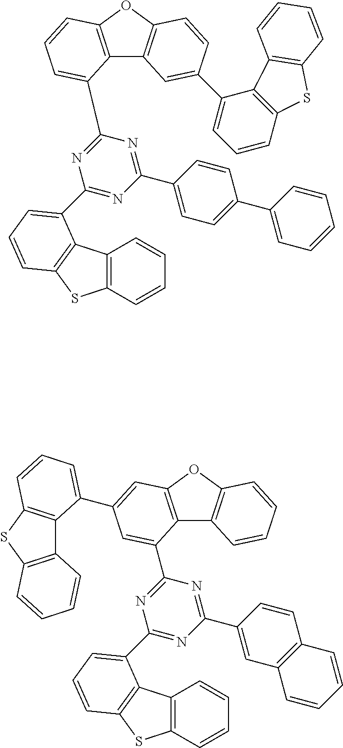 [SMILES: c1ccc(-c2ccc(-c3nc(-c4cccc5oc6ccc(-c7cccc8sc9ccccc9c78)cc6c45)nc(-c4cccc5sc6ccccc6c45)n3)cc2)cc1 ; c1ccc2cc(-c3nc(-c4cc(-c5cccc6sc7ccccc7c56)cc5oc6ccccc6c45)nc(-c4cccc5sc6ccccc6c45)n3)ccc2c1]

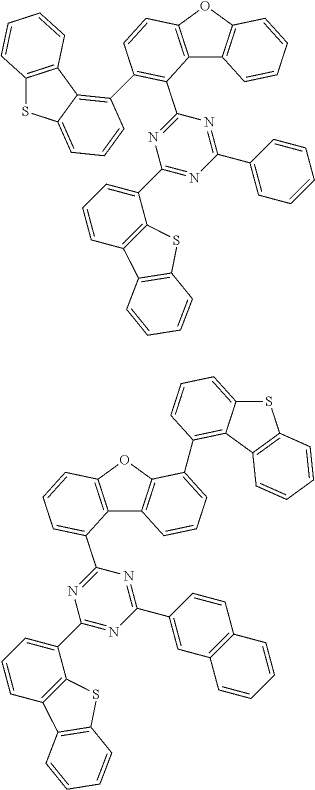 [SMILES: c1ccc(-c2nc(-c3cccc4c3sc3ccccc34)nc(-c3c(-c4cccc5sc6ccccc6c45)ccc4oc5ccccc5c34)n2)cc1 ; c1ccc2cc(-c3nc(-c4cccc5c4sc4ccccc45)nc(-c4cccc5oc6c(-c7cccc8sc9ccccc9c78)cccc6c45)n3)ccc2c1]

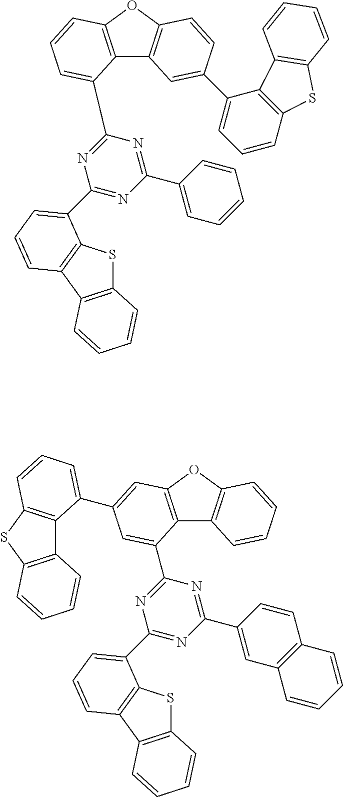 [SMILES: c1ccc(-c2nc(-c3cccc4c3sc3ccccc34)nc(-c3cccc4oc5ccc(-c6cccc7sc8ccccc8c67)cc5c34)n2)cc1 ; c1ccc2cc(-c3nc(-c4cccc5c4sc4ccccc45)nc(-c4cc(-c5cccc6sc7ccccc7c56)cc5oc6ccccc6c45)n3)ccc2c1]

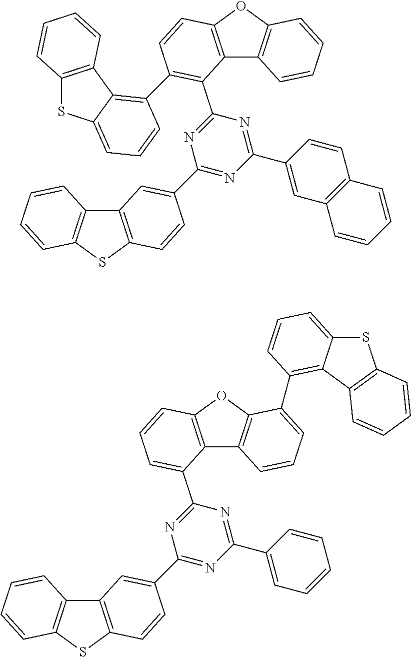 [SMILES: c1ccc2cc(-c3nc(-c4ccc5sc6ccccc6c5c4)nc(-c4c(-c5cccc6sc7ccccc7c56)ccc5oc6ccccc6c45)n3)ccc2c1 ; c1ccc(-c2nc(-c3ccc4sc5ccccc5c4c3)nc(-c3cccc4oc5c(-c6cccc7sc8ccccc8c67)cccc5c34)n2)cc1]

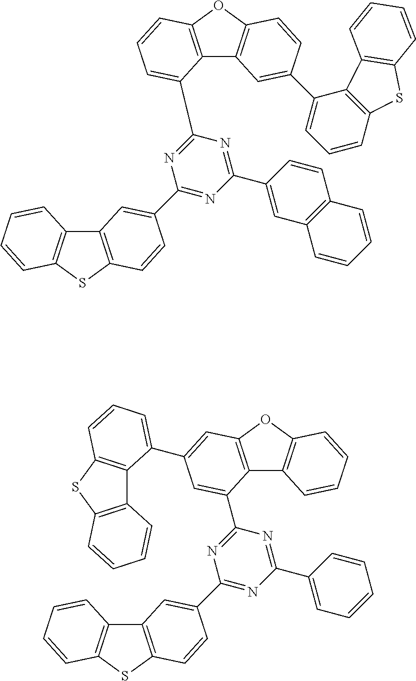 [SMILES: c1ccc2cc(-c3nc(-c4ccc5sc6ccccc6c5c4)nc(-c4cccc5oc6ccc(-c7cccc8sc9ccccc9c78)cc6c45)n3)ccc2c1 ; c1ccc(-c2nc(-c3ccc4sc5ccccc5c4c3)nc(-c3cc(-c4cccc5sc6ccccc6c45)cc4oc5ccccc5c34)n2)cc1]

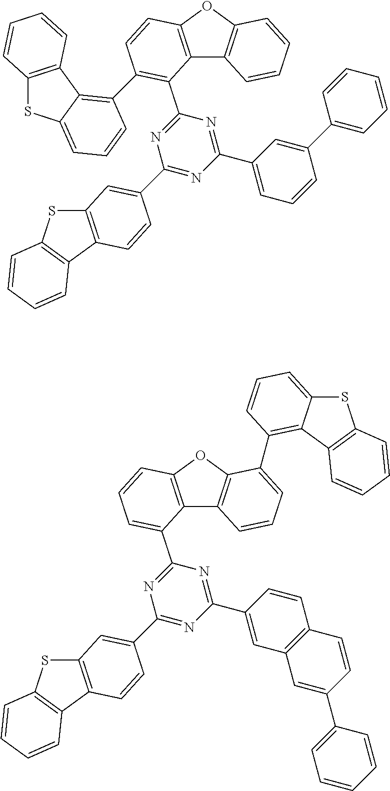 [SMILES: c1ccc(-c2cccc(-c3nc(-c4ccc5c(c4)sc4ccccc45)nc(-c4c(-c5cccc6sc7ccccc7c56)ccc5oc6ccccc6c45)n3)c2)cc1 ; c1ccc(-c2ccc3ccc(-c4nc(-c5ccc6c(c5)sc5ccccc56)nc(-c5cccc6oc7c(-c8cccc9sc%10ccccc%10c89)cccc7c56)n4)cc3c2)cc1]

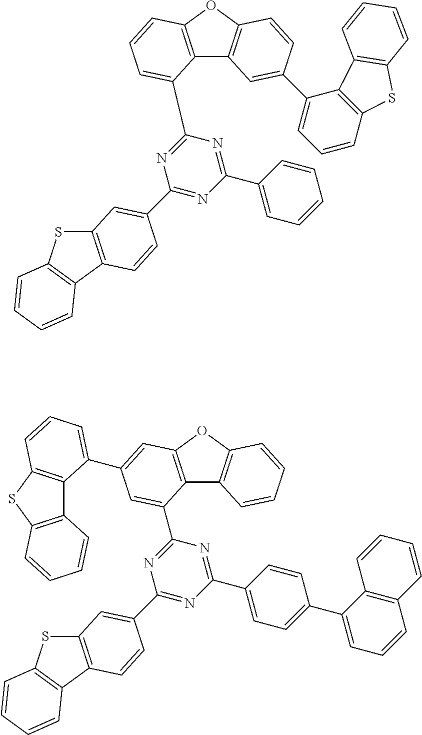 [SMILES: c1ccc(-c2nc(-c3ccc4c(c3)sc3ccccc34)nc(-c3cccc4oc5ccc(-c6cccc7sc8ccccc8c67)cc5c34)n2)cc1 ; c1ccc2c(-c3ccc(-c4nc(-c5ccc6c(c5)sc5ccccc56)nc(-c5cc(-c6cccc7sc8ccccc8c67)cc6oc7ccccc7c56)n4)cc3)cccc2c1]

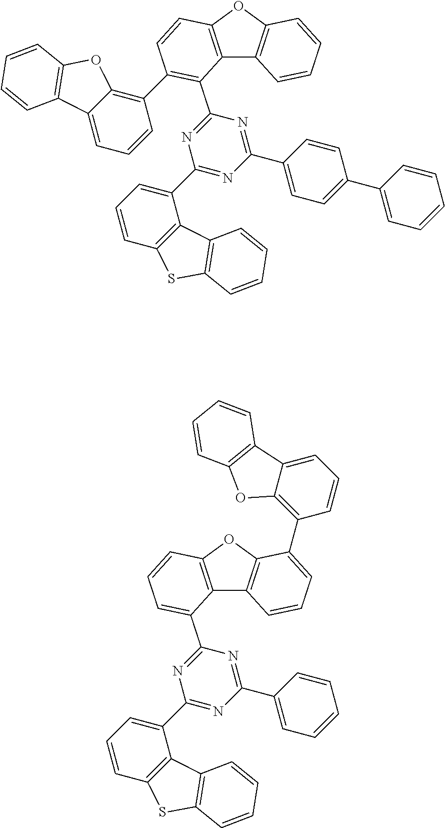 [SMILES: c1ccc(-c2ccc(-c3nc(-c4c(-c5cccc6c5oc5ccccc56)ccc5oc6ccccc6c45)nc(-c4cccc5sc6ccccc6c45)n3)cc2)cc1 ; c1ccc(-c2nc(-c3cccc4oc5c(-c6cccc7c6oc6ccccc67)cccc5c34)nc(-c3cccc4sc5ccccc5c34)n2)cc1]

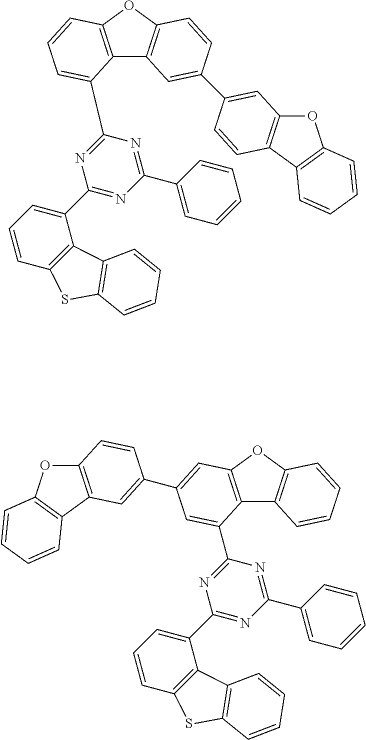 [SMILES: c1ccc(-c2nc(-c3cccc4oc5ccc(-c6ccc7c(c6)oc6ccccc67)cc5c34)nc(-c3cccc4sc5ccccc5c34)n2)cc1 ; c1ccc(-c2nc(-c3cc(-c4ccc5oc6ccccc6c5c4)cc4oc5ccccc5c34)nc(-c3cccc4sc5ccccc5c34)n2)cc1]

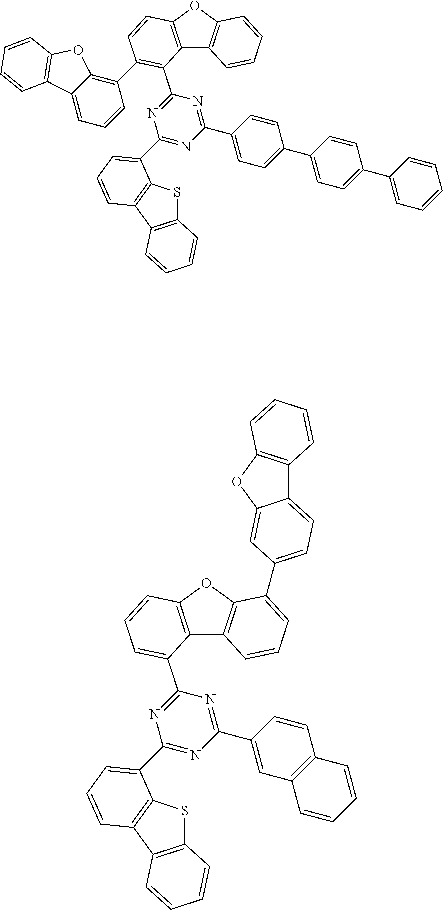 [SMILES: c1ccc(-c2ccc(-c3ccc(-c4nc(-c5cccc6c5sc5ccccc56)nc(-c5c(-c6cccc7c6oc6ccccc67)ccc6oc7ccccc7c56)n4)cc3)cc2)cc1 ; c1ccc2cc(-c3nc(-c4cccc5c4sc4ccccc45)nc(-c4cccc5oc6c(-c7ccc8c(c7)oc7ccccc78)cccc6c45)n3)ccc2c1]

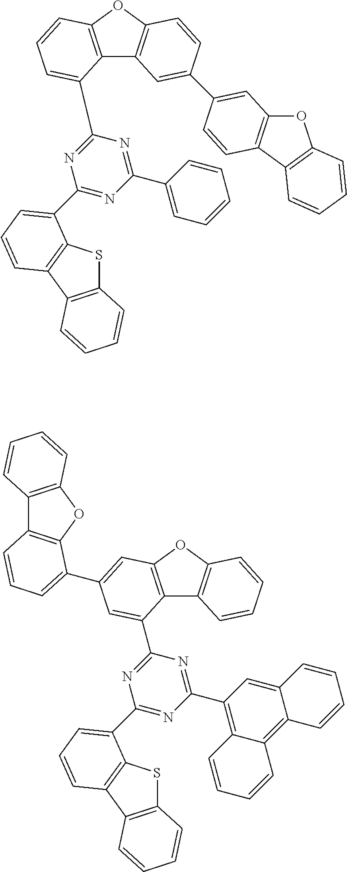 [SMILES: c1ccc(-c2nc(-c3cccc4c3sc3ccccc34)nc(-c3cccc4oc5ccc(-c6ccc7c(c6)oc6ccccc67)cc5c34)n2)cc1 ; c1ccc2c(c1)cc(-c1nc(-c3cccc4c3sc3ccccc34)nc(-c3cc(-c4cccc5c4oc4ccccc45)cc4oc5ccccc5c34)n1)c1ccccc12]

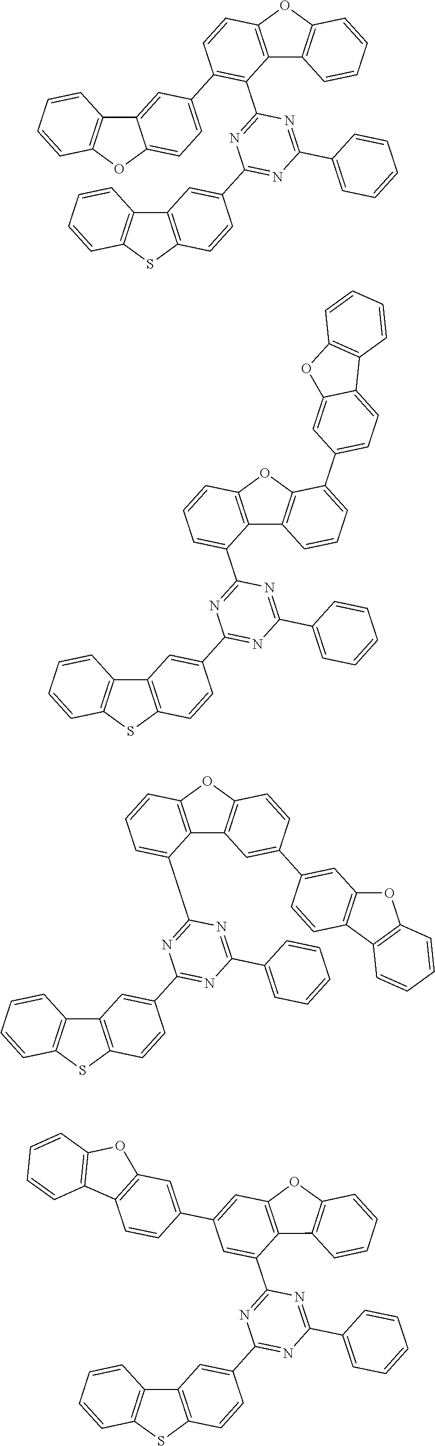 [SMILES: c1ccc(-c2nc(-c3ccc4sc5ccccc5c4c3)nc(-c3c(-c4ccc5oc6ccccc6c5c4)ccc4oc5ccccc5c34)n2)cc1 ; c1ccc(-c2nc(-c3ccc4sc5ccccc5c4c3)nc(-c3cccc4oc5c(-c6ccc7c(c6)oc6ccccc67)cccc5c34)n2)cc1 ; c1ccc(-c2nc(-c3ccc4sc5ccccc5c4c3)nc(-c3cccc4oc5ccc(-c6ccc7c(c6)oc6ccccc67)cc5c34)n2)cc1 ; c1ccc(-c2nc(-c3ccc4sc5ccccc5c4c3)nc(-c3cc(-c4ccc5c(c4)oc4ccccc45)cc4oc5ccccc5c34)n2)cc1]

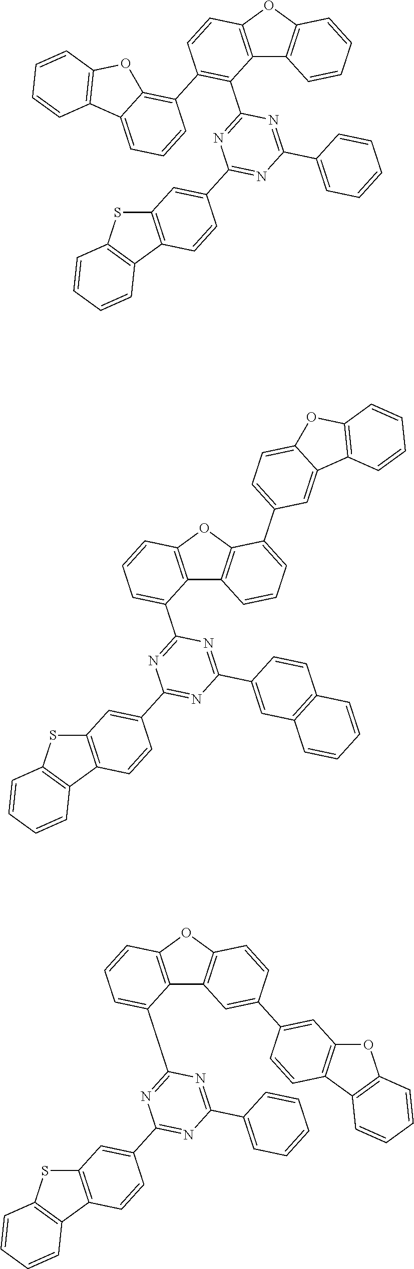 [SMILES: c1ccc(-c2nc(-c3ccc4c(c3)sc3ccccc34)nc(-c3c(-c4cccc5c4oc4ccccc45)ccc4oc5ccccc5c34)n2)cc1 ; c1ccc2cc(-c3nc(-c4ccc5c(c4)sc4ccccc45)nc(-c4cccc5oc6c(-c7ccc8oc9ccccc9c8c7)cccc6c45)n3)ccc2c1 ; c1ccc(-c2nc(-c3ccc4c(c3)sc3ccccc34)nc(-c3cccc4oc5ccc(-c6ccc7c(c6)oc6ccccc67)cc5c34)n2)cc1]

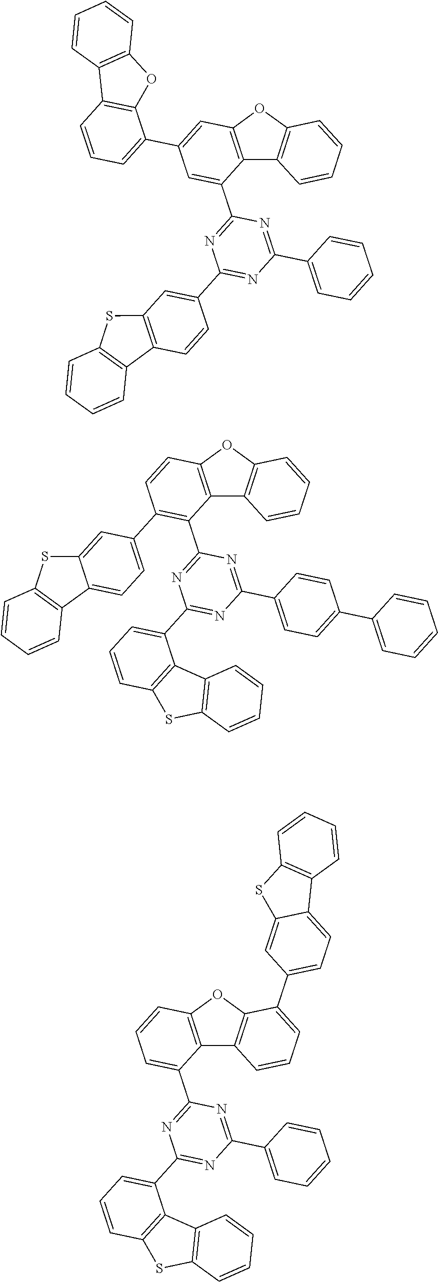 [SMILES: c1ccc(-c2nc(-c3ccc4c(c3)sc3ccccc34)nc(-c3cc(-c4cccc5c4oc4ccccc45)cc4oc5ccccc5c34)n2)cc1 ; c1ccc(-c2ccc(-c3nc(-c4c(-c5ccc6c(c5)sc5ccccc56)ccc5oc6ccccc6c45)nc(-c4cccc5sc6ccccc6c45)n3)cc2)cc1 ; c1ccc(-c2nc(-c3cccc4oc5c(-c6ccc7c(c6)sc6ccccc67)cccc5c34)nc(-c3cccc4sc5ccccc5c34)n2)cc1]

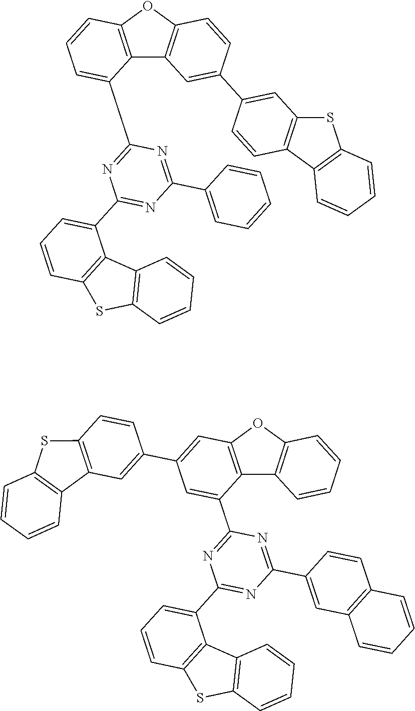 [SMILES: c1ccc(-c2nc(-c3cccc4oc5ccc(-c6ccc7c(c6)sc6ccccc67)cc5c34)nc(-c3cccc4sc5ccccc5c34)n2)cc1 ; c1ccc2cc(-c3nc(-c4cc(-c5ccc6sc7ccccc7c6c5)cc5oc6ccccc6c45)nc(-c4cccc5sc6ccccc6c45)n3)ccc2c1]

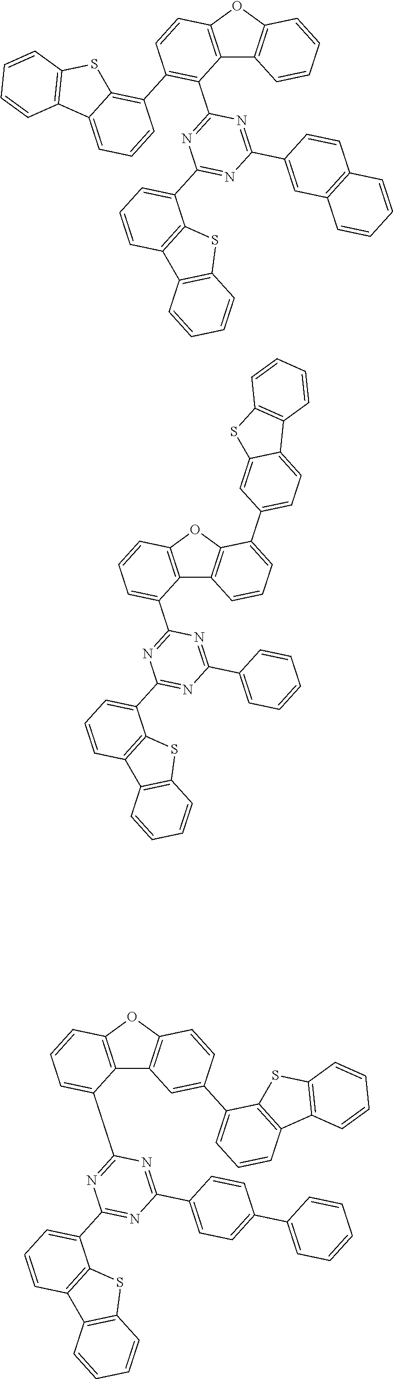 [SMILES: c1ccc2cc(-c3nc(-c4cccc5c4sc4ccccc45)nc(-c4c(-c5cccc6c5sc5ccccc56)ccc5oc6ccccc6c45)n3)ccc2c1 ; c1ccc(-c2nc(-c3cccc4c3sc3ccccc34)nc(-c3cccc4oc5c(-c6ccc7c(c6)sc6ccccc67)cccc5c34)n2)cc1 ; c1ccc(-c2ccc(-c3nc(-c4cccc5c4sc4ccccc45)nc(-c4cccc5oc6ccc(-c7cccc8c7sc7ccccc78)cc6c45)n3)cc2)cc1]

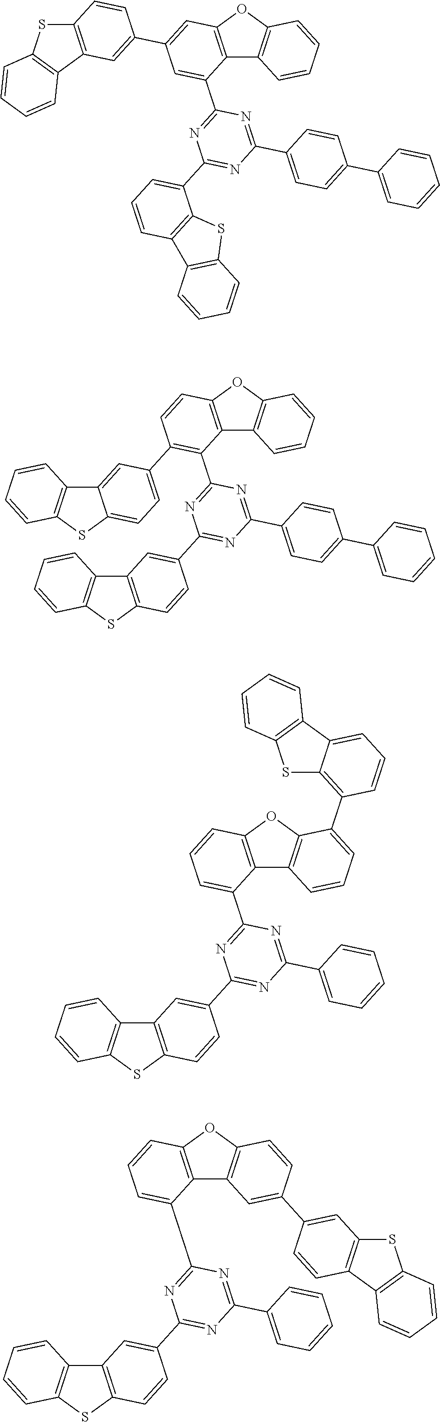 [SMILES: c1ccc(-c2ccc(-c3nc(-c4cccc5c4sc4ccccc45)nc(-c4cc(-c5ccc6sc7ccccc7c6c5)cc5oc6ccccc6c45)n3)cc2)cc1 ; c1ccc(-c2ccc(-c3nc(-c4ccc5sc6ccccc6c5c4)nc(-c4c(-c5ccc6sc7ccccc7c6c5)ccc5oc6ccccc6c45)n3)cc2)cc1 ; c1ccc(-c2nc(-c3ccc4sc5ccccc5c4c3)nc(-c3cccc4oc5c(-c6cccc7c6sc6ccccc67)cccc5c34)n2)cc1 ; c1ccc(-c2nc(-c3ccc4sc5ccccc5c4c3)nc(-c3cccc4oc5ccc(-c6ccc7c(c6)sc6ccccc67)cc5c34)n2)cc1]

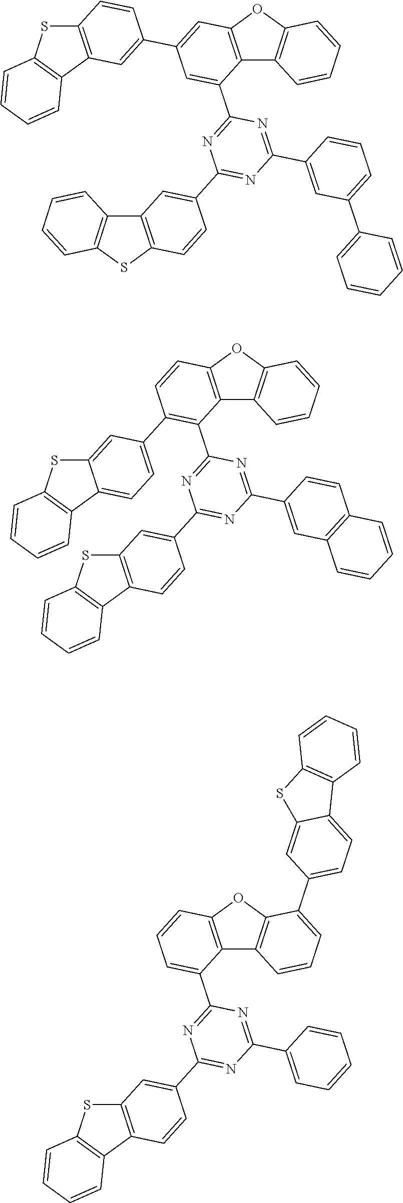 [SMILES: c1ccc(-c2cccc(-c3nc(-c4ccc5sc6ccccc6c5c4)nc(-c4cc(-c5ccc6sc7ccccc7c6c5)cc5oc6ccccc6c45)n3)c2)cc1 ; c1ccc2cc(-c3nc(-c4ccc5c(c4)sc4ccccc45)nc(-c4c(-c5ccc6c(c5)sc5ccccc56)ccc5oc6ccccc6c45)n3)ccc2c1 ; c1ccc(-c2nc(-c3ccc4c(c3)sc3ccccc34)nc(-c3cccc4oc5c(-c6ccc7c(c6)sc6ccccc67)cccc5c34)n2)cc1]

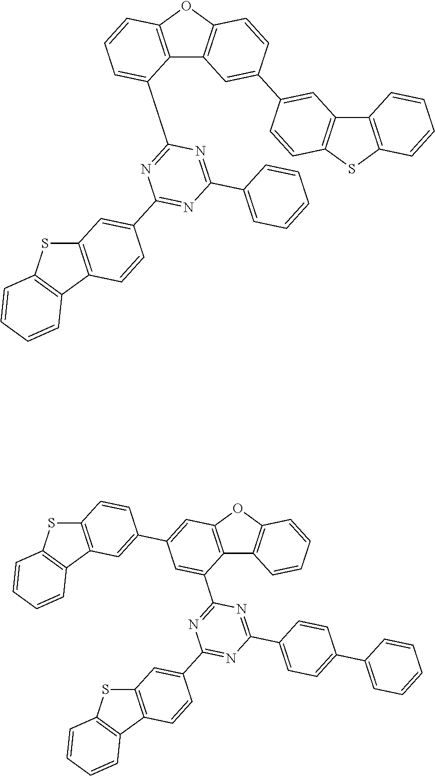 [SMILES: c1ccc(-c2nc(-c3ccc4c(c3)sc3ccccc34)nc(-c3cccc4oc5ccc(-c6ccc7sc8ccccc8c7c6)cc5c34)n2)cc1 ; c1ccc(-c2ccc(-c3nc(-c4ccc5c(c4)sc4ccccc45)nc(-c4cc(-c5ccc6sc7ccccc7c6c5)cc5oc6ccccc6c45)n3)cc2)cc1]

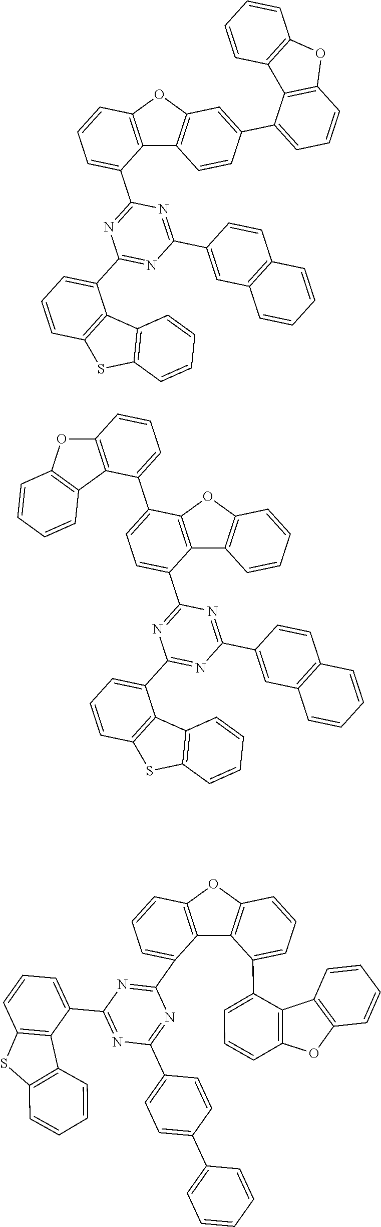

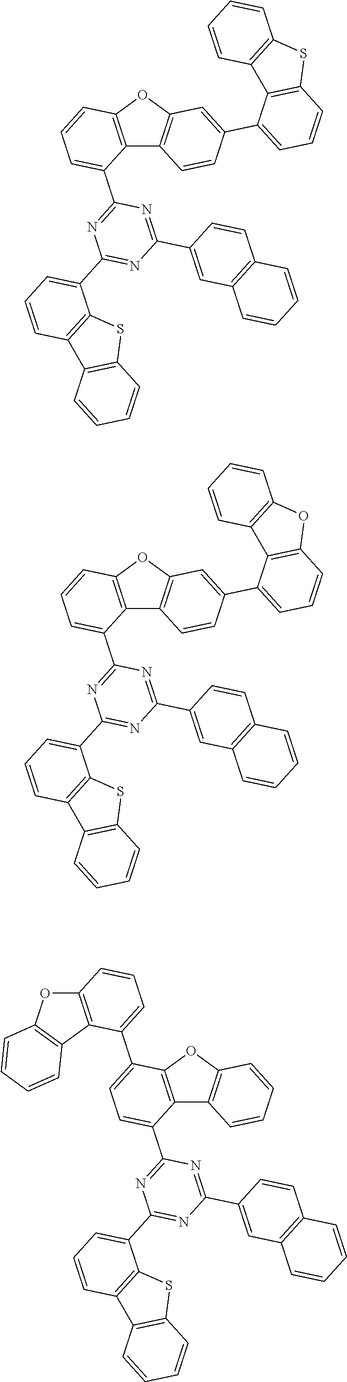 [SMILES: c1ccc2cc(-c3nc(-c4cccc5c4sc4ccccc45)nc(-c4cccc5oc6cc(-c7cccc8sc9ccccc9c78)ccc6c45)n3)ccc2c1 ; c1ccc2cc(-c3nc(-c4cccc5c4sc4ccccc45)nc(-c4cccc5oc6cc(-c7cccc8oc9ccccc9c78)ccc6c45)n3)ccc2c1 ; c1ccc2cc(-c3nc(-c4cccc5c4sc4ccccc45)nc(-c4ccc(-c5cccc6oc7ccccc7c56)c5oc6ccccc6c45)n3)ccc2c1]

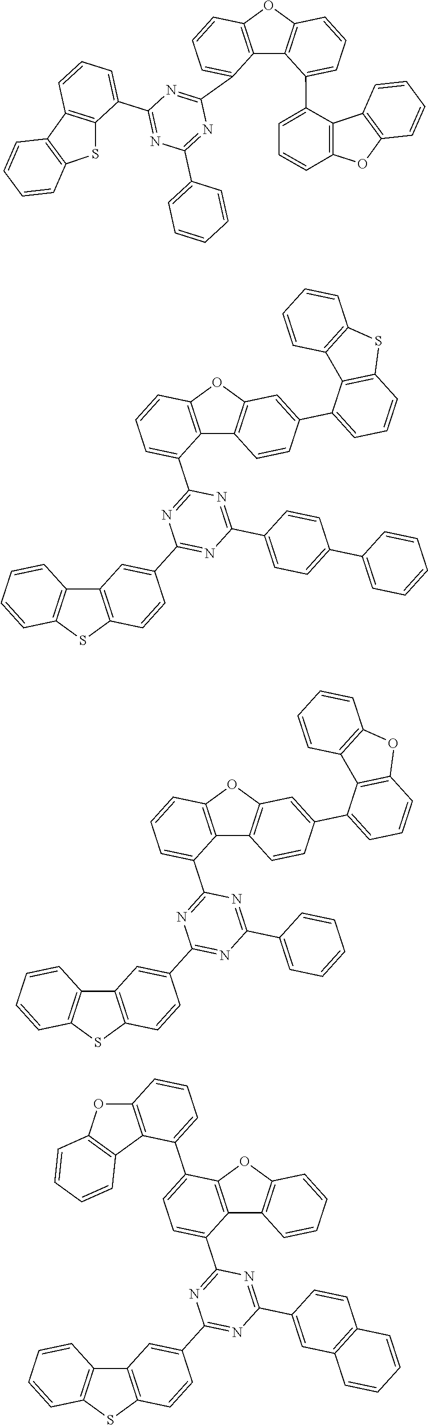 [SMILES: c1ccc(-c2nc(-c3cccc4c3sc3ccccc34)nc(-c3cccc4oc5cccc(-c6cccc7oc8ccccc8c67)c5c34)n2)cc1 ; c1ccc(-c2ccc(-c3nc(-c4ccc5sc6ccccc6c5c4)nc(-c4cccc5oc6cc(-c7cccc8sc9ccccc9c78)ccc6c45)n3)cc2)cc1 ; c1ccc(-c2nc(-c3ccc4sc5ccccc5c4c3)nc(-c3cccc4oc5cc(-c6cccc7oc8ccccc8c67)ccc5c34)n2)cc1 ; c1ccc2cc(-c3nc(-c4ccc5sc6ccccc6c5c4)nc(-c4ccc(-c5cccc6oc7ccccc7c56)c5oc6ccccc6c45)n3)ccc2c1]

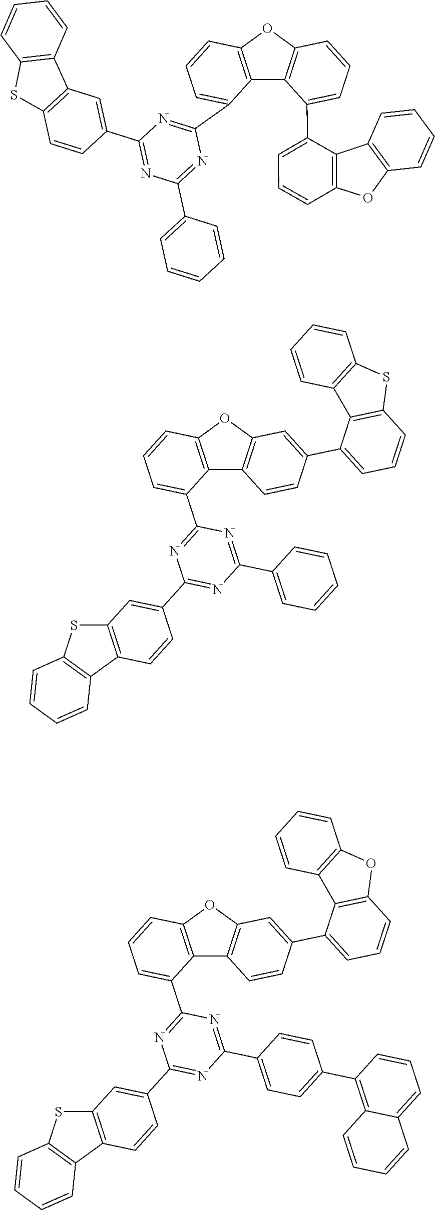 [SMILES: c1ccc(-c2nc(-c3ccc4sc5ccccc5c4c3)nc(-c3cccc4oc5cccc(-c6cccc7oc8ccccc8c67)c5c34)n2)cc1 ; c1ccc(-c2nc(-c3ccc4c(c3)sc3ccccc34)nc(-c3cccc4oc5cc(-c6cccc7sc8ccccc8c67)ccc5c34)n2)cc1 ; c1ccc2c(-c3ccc(-c4nc(-c5ccc6c(c5)sc5ccccc56)nc(-c5cccc6oc7cc(-c8cccc9oc%10ccccc%10c89)ccc7c56)n4)cc3)cccc2c1]

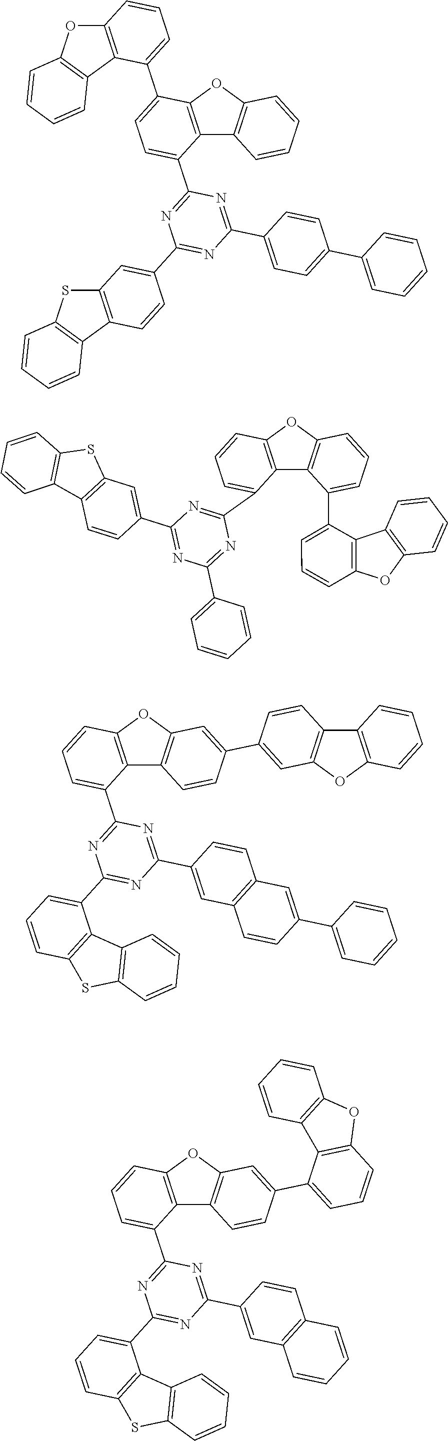

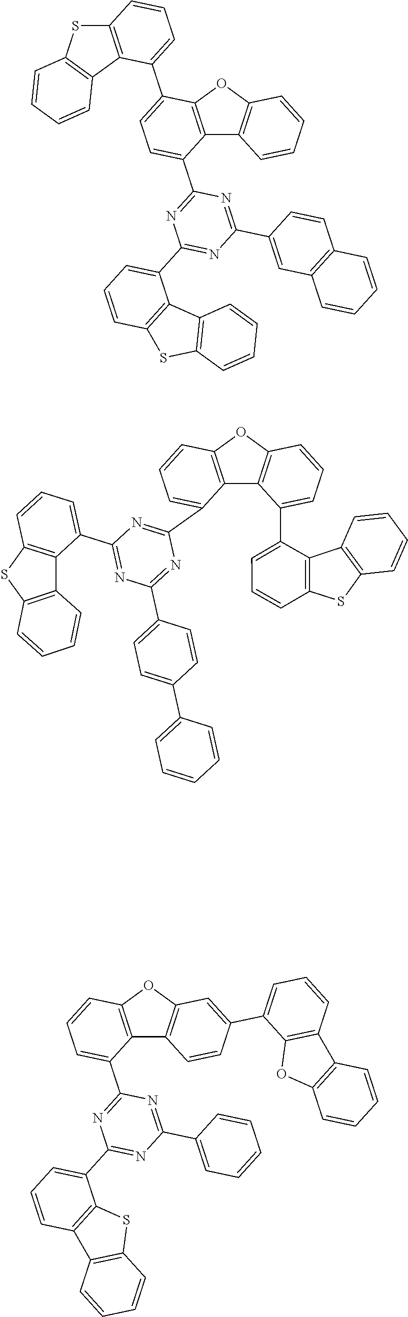 [SMILES: c1ccc2cc(-c3nc(-c4cccc5sc6ccccc6c45)nc(-c4ccc(-c5cccc6sc7ccccc7c56)c5oc6ccccc6c45)n3)ccc2c1 ; c1ccc(-c2ccc(-c3nc(-c4cccc5sc6ccccc6c45)nc(-c4cccc5oc6cccc(-c7cccc8sc9ccccc9c78)c6c45)n3)cc2)cc1 ; c1ccc(-c2nc(-c3cccc4c3sc3ccccc34)nc(-c3cccc4oc5cc(-c6cccc7c6oc6ccccc67)ccc5c34)n2)cc1]

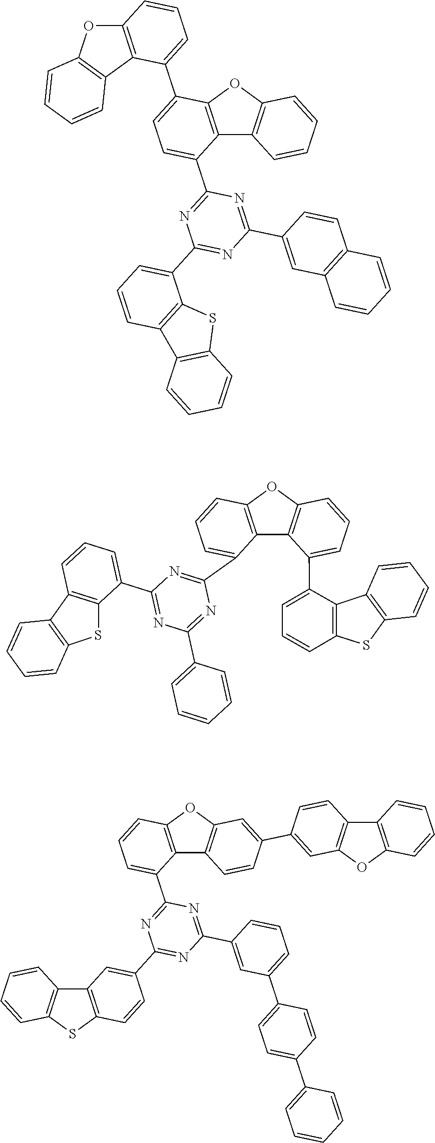 [SMILES: c1ccc2cc(-c3nc(-c4cccc5c4sc4ccccc45)nc(-c4ccc(-c5cccc6oc7ccccc7c56)c5oc6ccccc6c45)n3)ccc2c1 ; c1ccc(-c2nc(-c3cccc4c3sc3ccccc34)nc(-c3cccc4oc5cccc(-c6cccc7sc8ccccc8c67)c5c34)n2)cc1 ; c1ccc(-c2ccc(-c3cccc(-c4nc(-c5ccc6sc7ccccc7c6c5)nc(-c5cccc6oc7cc(-c8ccc9c(c8)oc8ccccc89)ccc7c56)n4)c3)cc2)cc1]

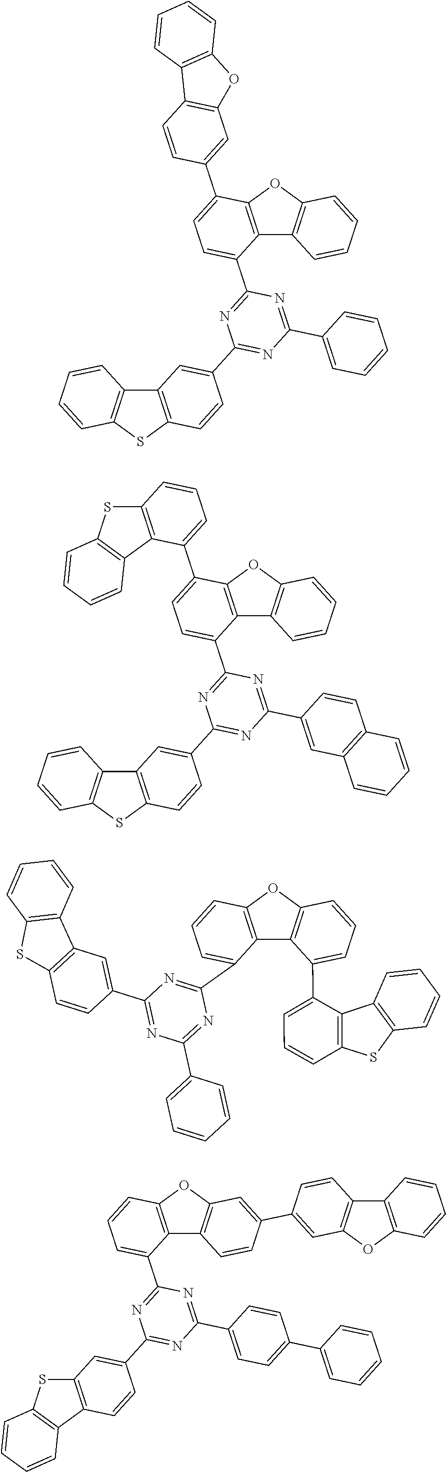

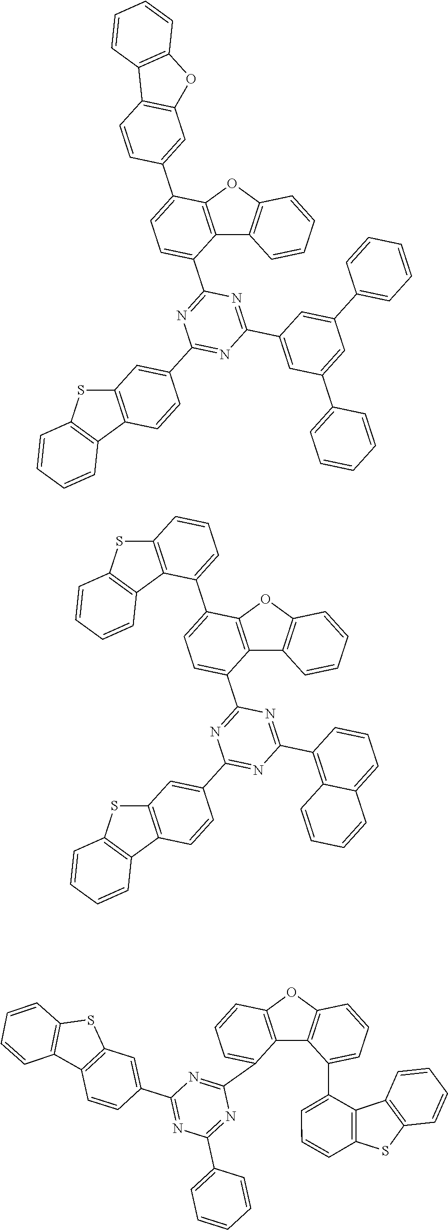 [SMILES: c1ccc(-c2cc(-c3ccccc3)cc(-c3nc(-c4ccc5c(c4)sc4ccccc45)nc(-c4ccc(-c5ccc6c(c5)oc5ccccc56)c5oc6ccccc6c45)n3)c2)cc1 ; c1ccc2c(-c3nc(-c4ccc5c(c4)sc4ccccc45)nc(-c4ccc(-c5cccc6sc7ccccc7c56)c5oc6ccccc6c45)n3)cccc2c1 ; c1ccc(-c2nc(-c3ccc4c(c3)sc3ccccc34)nc(-c3cccc4oc5cccc(-c6cccc7sc8ccccc8c67)c5c34)n2)cc1]

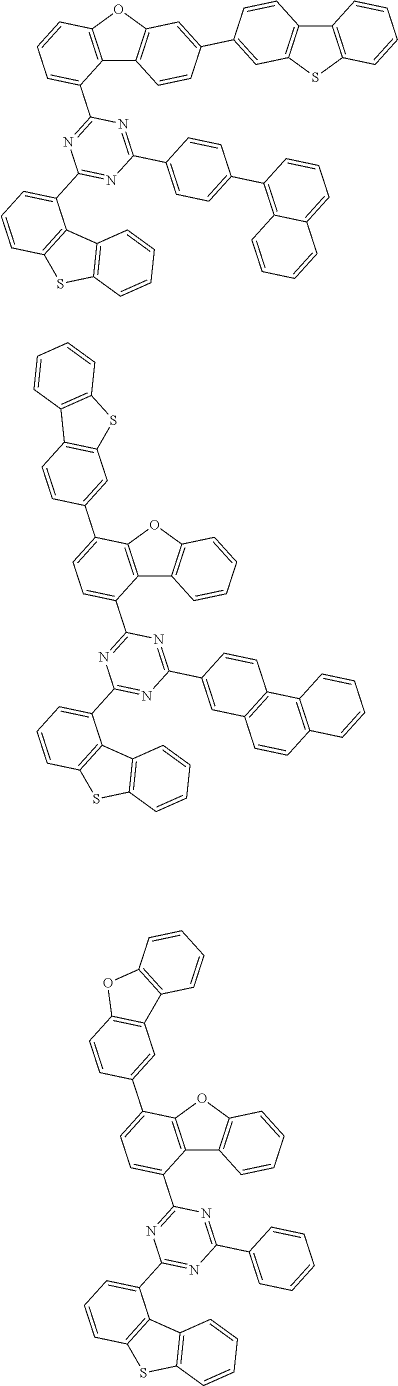 [SMILES: c1ccc2c(-c3ccc(-c4nc(-c5cccc6oc7cc(-c8ccc9c(c8)sc8ccccc89)ccc7c56)nc(-c5cccc6sc7ccccc7c56)n4)cc3)cccc2c1 ; c1ccc2c(c1)ccc1cc(-c3nc(-c4cccc5sc6ccccc6c45)nc(-c4ccc(-c5ccc6c(c5)sc5ccccc56)c5oc6ccccc6c45)n3)ccc12 ; c1ccc(-c2nc(-c3cccc4sc5ccccc5c34)nc(-c3ccc(-c4ccc5oc6ccccc6c5c4)c4oc5ccccc5c34)n2)cc1]

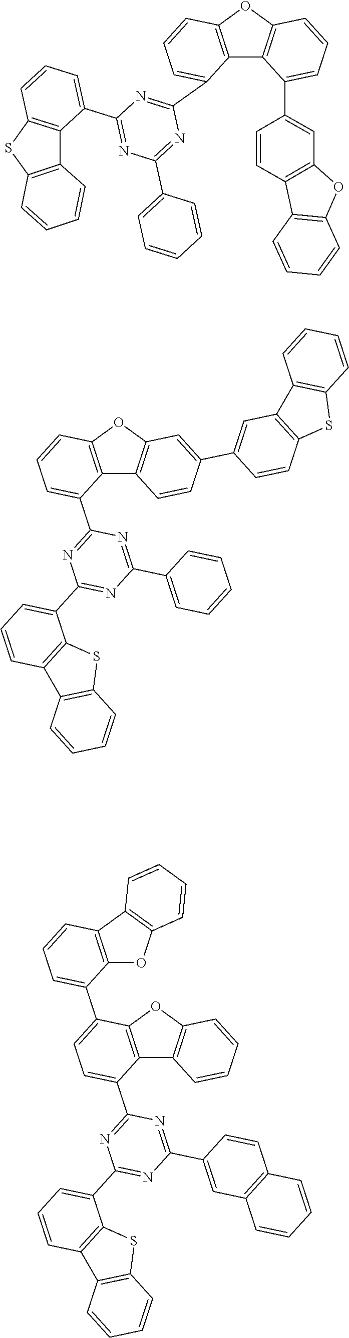 [SMILES: c1ccc(-c2nc(-c3cccc4sc5ccccc5c34)nc(-c3cccc4oc5cccc(-c6ccc7c(c6)oc6ccccc67)c5c34)n2)cc1 ; c1ccc(-c2nc(-c3cccc4c3sc3ccccc34)nc(-c3cccc4oc5cc(-c6ccc7sc8ccccc8c7c6)ccc5c34)n2)cc1 ; c1ccc2cc(-c3nc(-c4cccc5c4sc4ccccc45)nc(-c4ccc(-c5cccc6c5oc5ccccc56)c5oc6ccccc6c45)n3)ccc2c1]

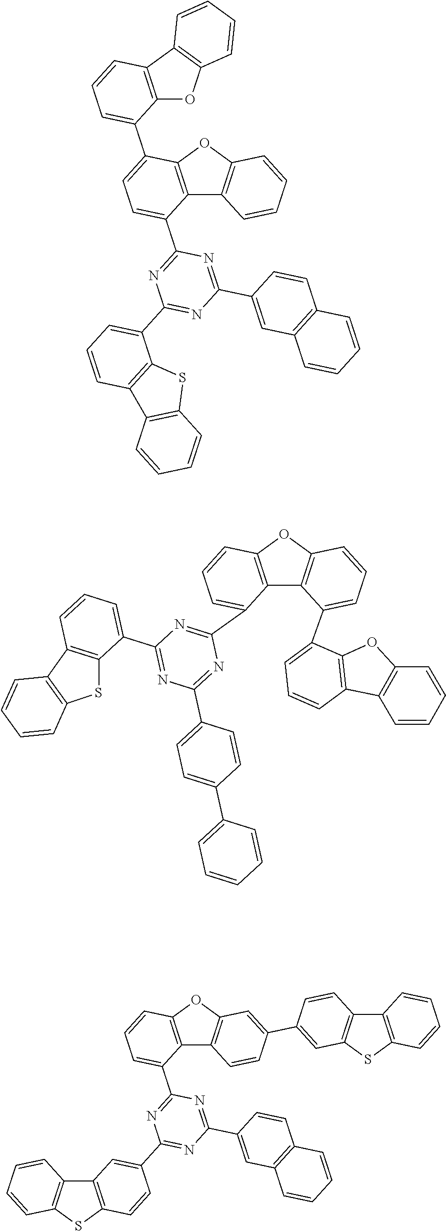 [SMILES: c1ccc2cc(-c3nc(-c4cccc5c4sc4ccccc45)nc(-c4ccc(-c5cccc6c5oc5ccccc56)c5oc6ccccc6c45)n3)ccc2c1 ; c1ccc(-c2ccc(-c3nc(-c4cccc5c4sc4ccccc45)nc(-c4cccc5oc6cccc(-c7cccc8c7oc7ccccc78)c6c45)n3)cc2)cc1 ; c1ccc2cc(-c3nc(-c4ccc5sc6ccccc6c5c4)nc(-c4cccc5oc6cc(-c7ccc8c(c7)sc7ccccc78)ccc6c45)n3)ccc2c1]

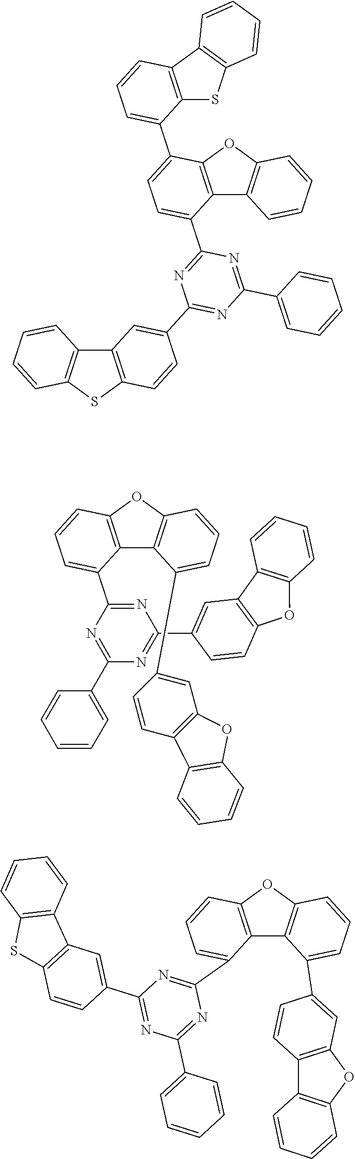 [SMILES: c1ccc(-c2nc(-c3ccc4sc5ccccc5c4c3)nc(-c3ccc(-c4cccc5c4sc4ccccc45)c4oc5ccccc5c34)n2)cc1 ; c1ccc(-c2nc(-c3ccc4oc5ccccc5c4c3)nc(-c3cccc4oc5cccc(-c6ccc7c(c6)oc6ccccc67)c5c34)n2)cc1 ; c1ccc(-c2nc(-c3ccc4sc5ccccc5c4c3)nc(-c3cccc4oc5cccc(-c6ccc7c(c6)oc6ccccc67)c5c34)n2)cc1]

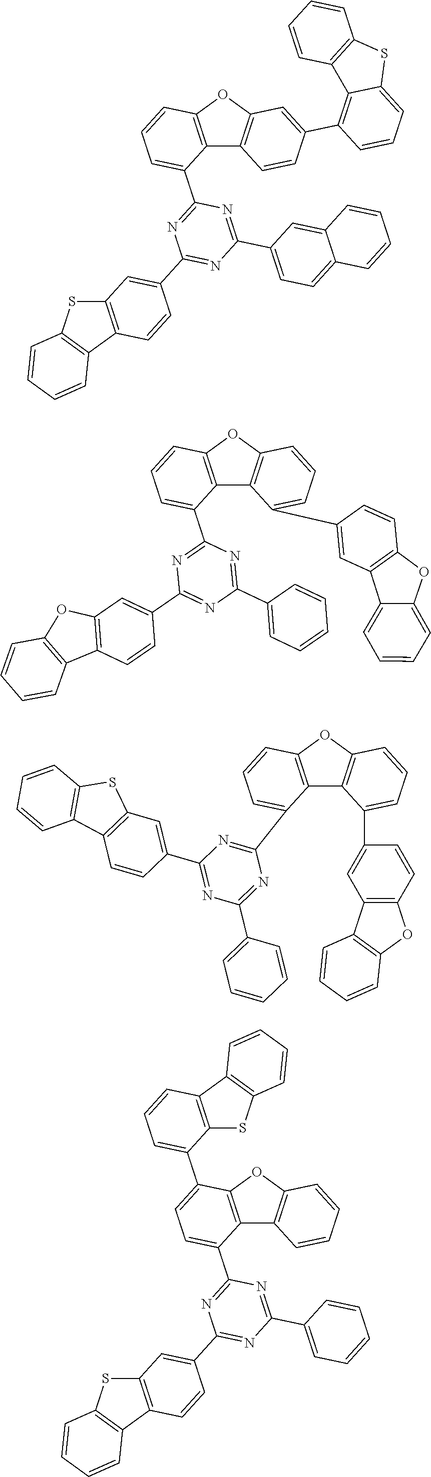

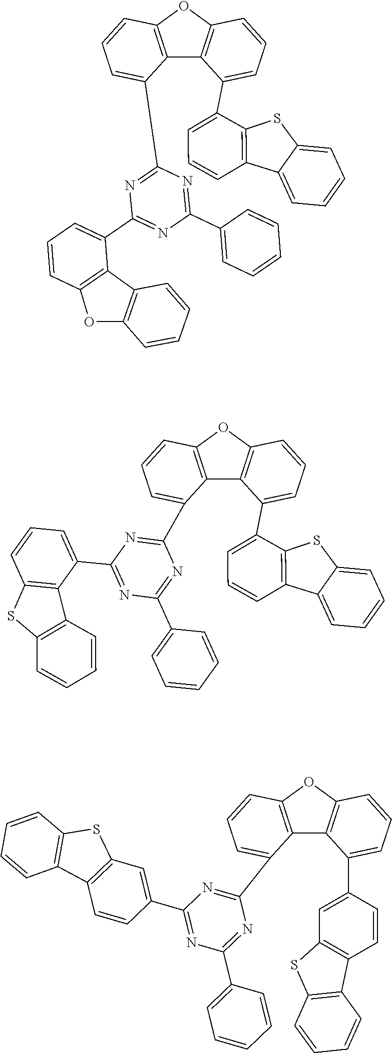 [SMILES: c1ccc(-c2nc(-c3cccc4oc5ccccc5c34)nc(-c3cccc4oc5cccc(-c6cccc7c6sc6ccccc67)c5c34)n2)cc1 ; c1ccc(-c2nc(-c3cccc4sc5ccccc5c34)nc(-c3cccc4oc5cccc(-c6cccc7c6sc6ccccc67)c5c34)n2)cc1 ; c1ccc(-c2nc(-c3ccc4c(c3)sc3ccccc34)nc(-c3cccc4oc5cccc(-c6ccc7c(c6)sc6ccccc67)c5c34)n2)cc1]

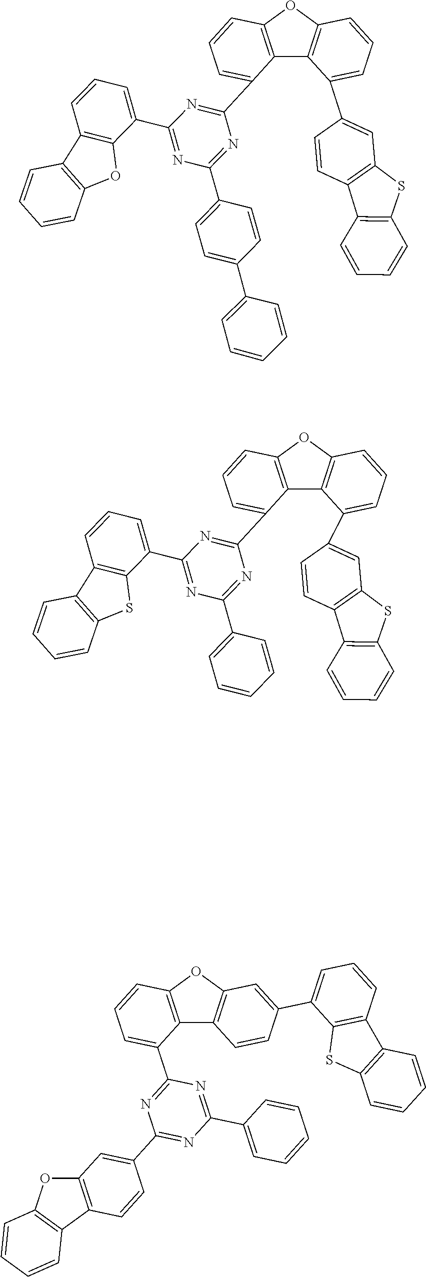 [SMILES: c1ccc(-c2ccc(-c3nc(-c4cccc5c4oc4ccccc45)nc(-c4cccc5oc6cccc(-c7ccc8c(c7)sc7ccccc78)c6c45)n3)cc2)cc1 ; c1ccc(-c2nc(-c3cccc4c3sc3ccccc34)nc(-c3cccc4oc5cccc(-c6ccc7c(c6)sc6ccccc67)c5c34)n2)cc1 ; c1ccc(-c2nc(-c3ccc4c(c3)oc3ccccc34)nc(-c3cccc4oc5cc(-c6cccc7c6sc6ccccc67)ccc5c34)n2)cc1]

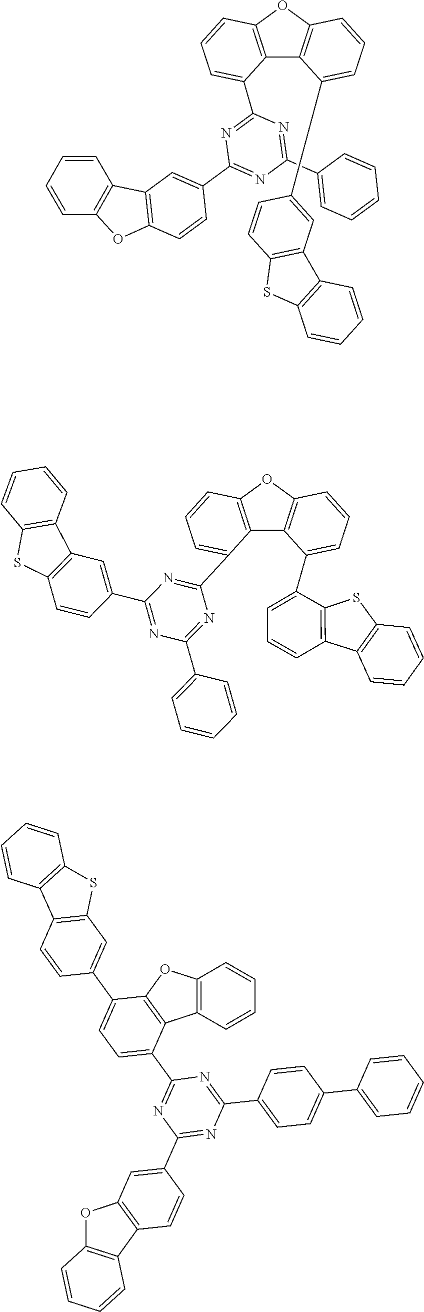 [SMILES: c1ccc(-c2nc(-c3ccc4oc5ccccc5c4c3)nc(-c3cccc4oc5cccc(-c6ccc7sc8ccccc8c7c6)c5c34)n2)cc1 ; c1ccc(-c2nc(-c3ccc4sc5ccccc5c4c3)nc(-c3cccc4oc5cccc(-c6cccc7c6sc6ccccc67)c5c34)n2)cc1 ; c1ccc(-c2ccc(-c3nc(-c4ccc5c(c4)oc4ccccc45)nc(-c4ccc(-c5ccc6c(c5)sc5ccccc56)c5oc6ccccc6c45)n3)cc2)cc1]

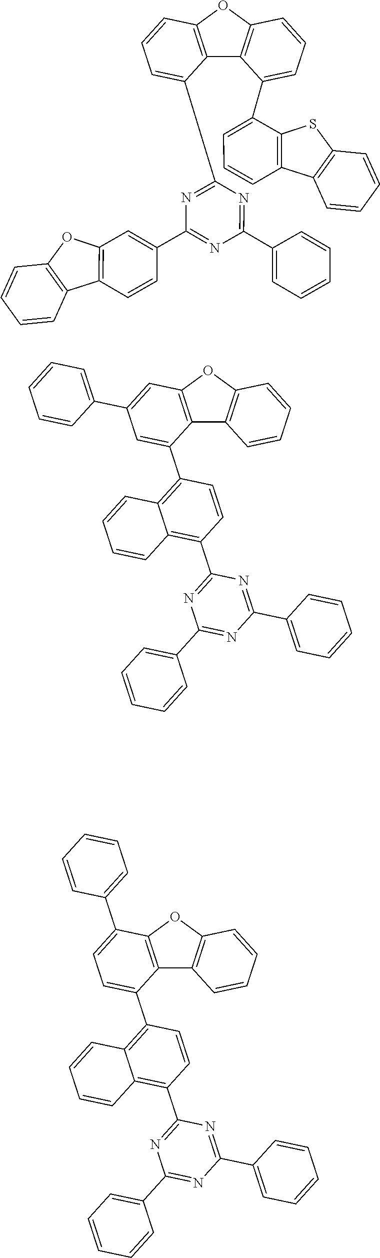

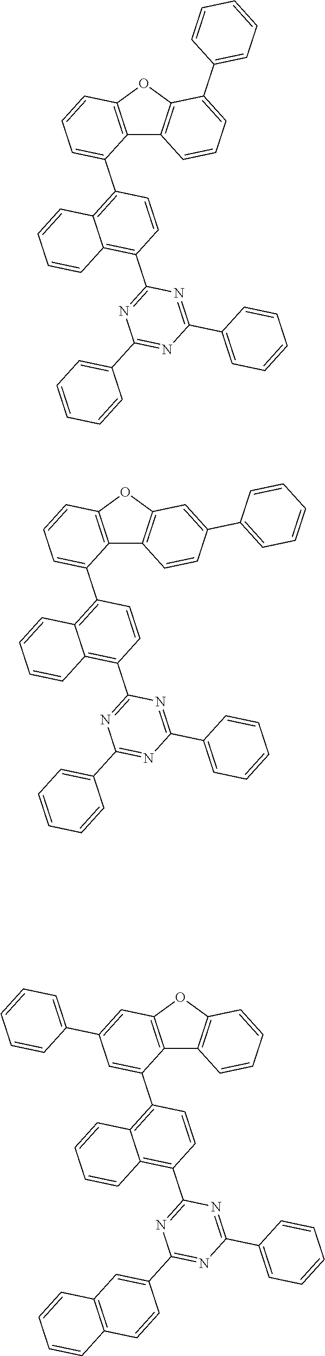 [SMILES: c1ccc(-c2nc(-c3ccccc3)nc(-c3ccc(-c4cccc5oc6c(-c7ccccc7)cccc6c45)c4ccccc34)n2)cc1 ; c1ccc(-c2ccc3c(c2)oc2cccc(-c4ccc(-c5nc(-c6ccccc6)nc(-c6ccccc6)n5)c5ccccc45)c23)cc1 ; c1ccc(-c2cc(-c3ccc(-c4nc(-c5ccccc5)nc(-c5ccc6ccccc6c5)n4)c4ccccc34)c3c(c2)oc2ccccc23)cc1]

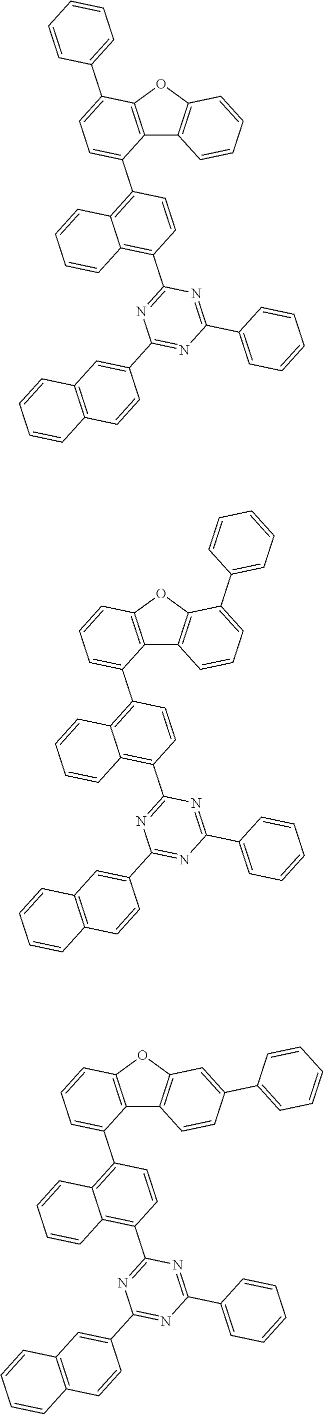 [SMILES: c1ccc(-c2nc(-c3ccc4ccccc4c3)nc(-c3ccc(-c4ccc(-c5ccccc5)c5oc6ccccc6c45)c4ccccc34)n2)cc1 ; c1ccc(-c2nc(-c3ccc4ccccc4c3)nc(-c3ccc(-c4cccc5oc6c(-c7ccccc7)cccc6c45)c4ccccc34)n2)cc1 ; c1ccc(-c2ccc3c(c2)oc2cccc(-c4ccc(-c5nc(-c6ccccc6)nc(-c6ccc7ccccc7c6)n5)c5ccccc45)c23)cc1]

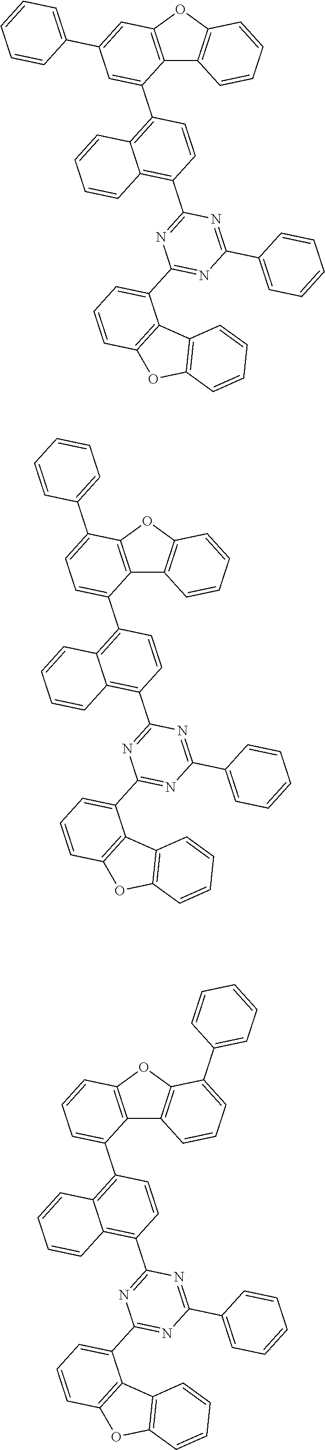 [SMILES: c1ccc(-c2cc(-c3ccc(-c4nc(-c5ccccc5)nc(-c5cccc6oc7ccccc7c56)n4)c4ccccc34)c3c(c2)oc2ccccc23)cc1 ; c1ccc(-c2nc(-c3ccc(-c4ccc(-c5ccccc5)c5oc6ccccc6c45)c4ccccc34)nc(-c3cccc4oc5ccccc5c34)n2)cc1 ; c1ccc(-c2nc(-c3ccc(-c4cccc5oc6c(-c7ccccc7)cccc6c45)c4ccccc34)nc(-c3cccc4oc5ccccc5c34)n2)cc1]

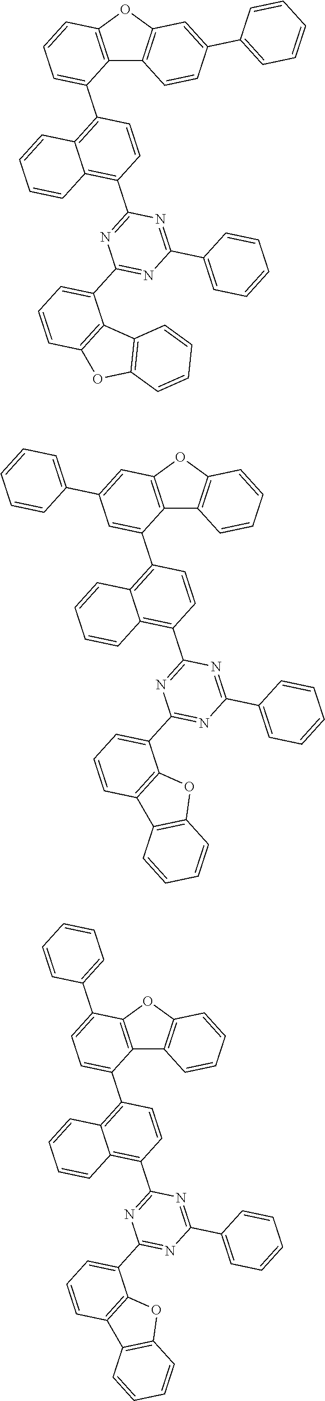 [SMILES: c1ccc(-c2ccc3c(c2)oc2cccc(-c4ccc(-c5nc(-c6ccccc6)nc(-c6cccc7oc8ccccc8c67)n5)c5ccccc45)c23)cc1 ; c1ccc(-c2cc(-c3ccc(-c4nc(-c5ccccc5)nc(-c5cccc6c5oc5ccccc56)n4)c4ccccc34)c3c(c2)oc2ccccc23)cc1 ; c1ccc(-c2nc(-c3ccc(-c4ccc(-c5ccccc5)c5oc6ccccc6c45)c4ccccc34)nc(-c3cccc4c3oc3ccccc34)n2)cc1]

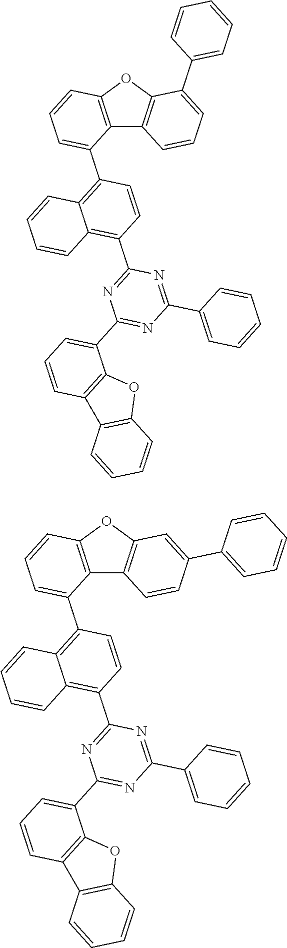

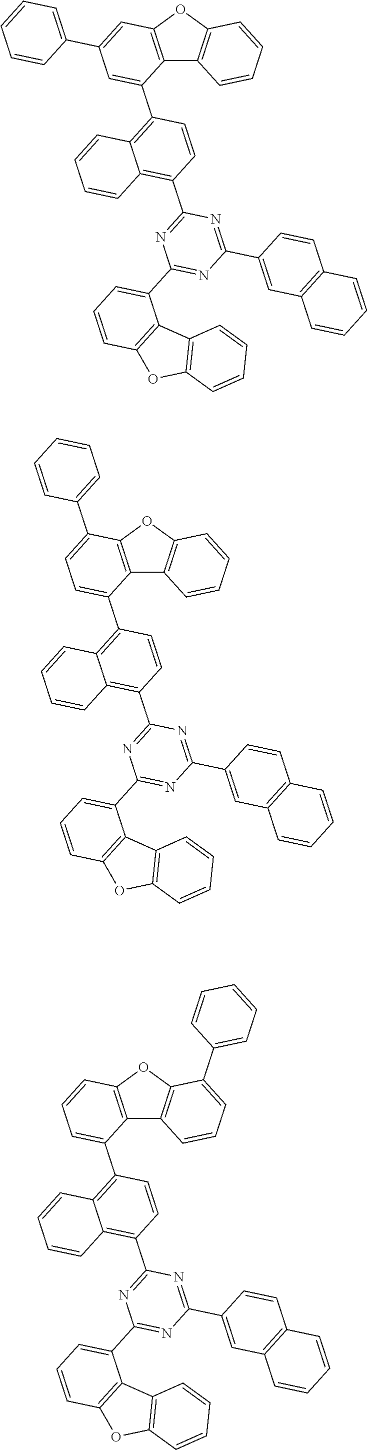 [SMILES: c1ccc(-c2cc(-c3ccc(-c4nc(-c5ccc6ccccc6c5)nc(-c5cccc6oc7ccccc7c56)n4)c4ccccc34)c3c(c2)oc2ccccc23)cc1 ; c1ccc(-c2ccc(-c3ccc(-c4nc(-c5ccc6ccccc6c5)nc(-c5cccc6oc7ccccc7c56)n4)c4ccccc34)c3c2oc2ccccc23)cc1 ; c1ccc(-c2cccc3c2oc2cccc(-c4ccc(-c5nc(-c6ccc7ccccc7c6)nc(-c6cccc7oc8ccccc8c67)n5)c5ccccc45)c23)cc1]

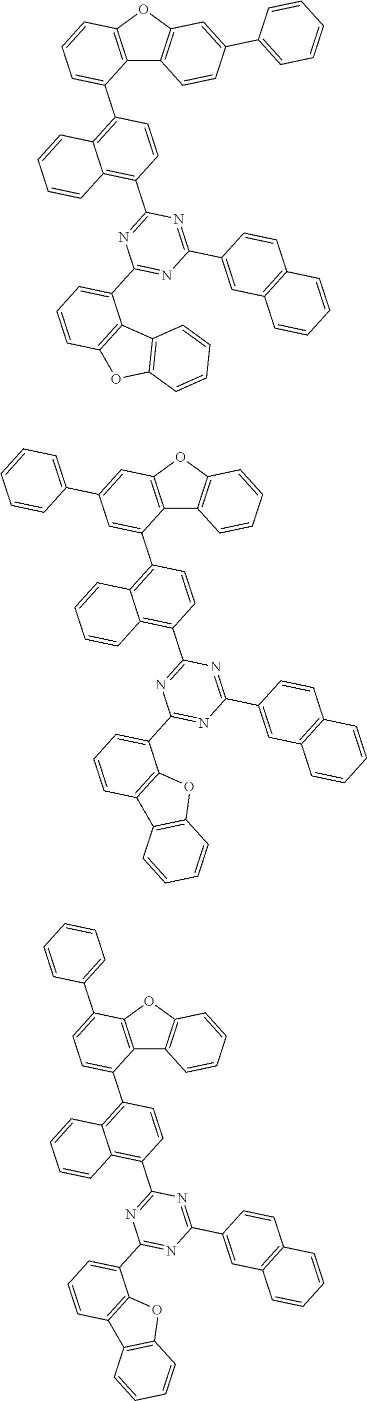 [SMILES: c1ccc(-c2ccc3c(c2)oc2cccc(-c4ccc(-c5nc(-c6ccc7ccccc7c6)nc(-c6cccc7oc8ccccc8c67)n5)c5ccccc45)c23)cc1 ; c1ccc(-c2cc(-c3ccc(-c4nc(-c5ccc6ccccc6c5)nc(-c5cccc6c5oc5ccccc56)n4)c4ccccc34)c3c(c2)oc2ccccc23)cc1 ; c1ccc(-c2ccc(-c3ccc(-c4nc(-c5ccc6ccccc6c5)nc(-c5cccc6c5oc5ccccc56)n4)c4ccccc34)c3c2oc2ccccc23)cc1]

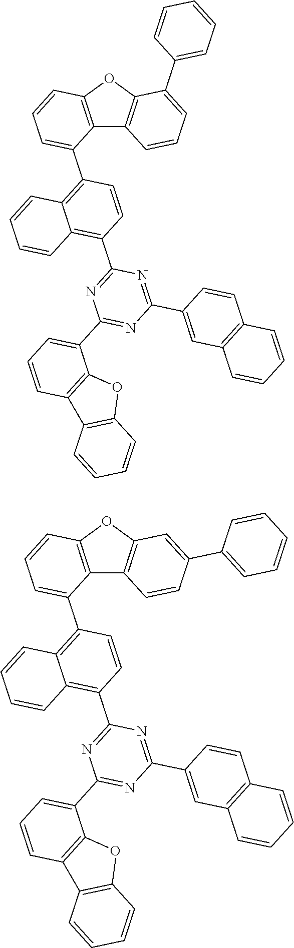

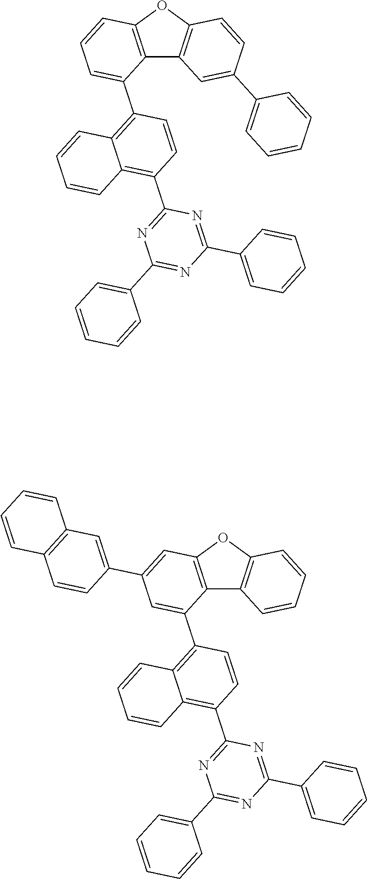 [SMILES: c1ccc(-c2ccc3oc4cccc(-c5ccc(-c6nc(-c7ccccc7)nc(-c7ccccc7)n6)c6ccccc56)c4c3c2)cc1 ; c1ccc(-c2nc(-c3ccccc3)nc(-c3ccc(-c4cc(-c5ccc6ccccc6c5)cc5oc6ccccc6c45)c4ccccc34)n2)cc1]

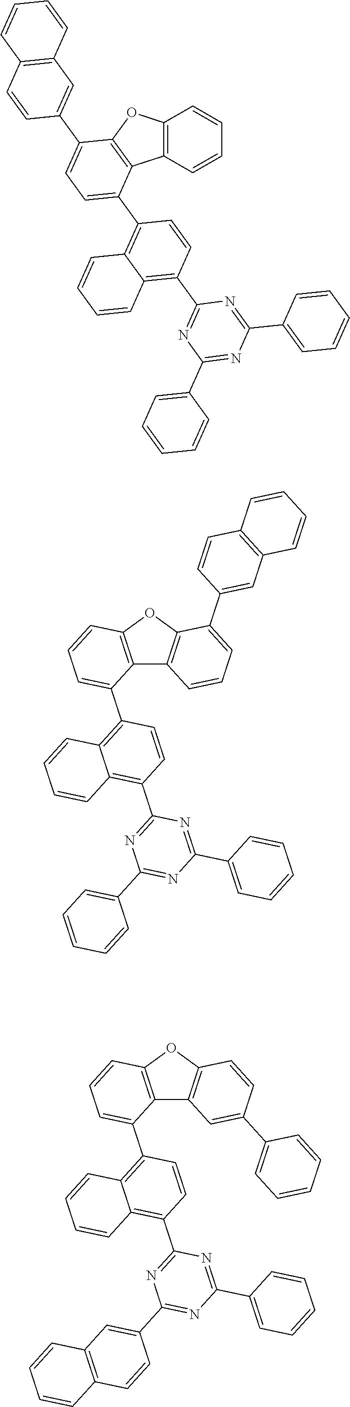 [SMILES: c1ccc(-c2nc(-c3ccccc3)nc(-c3ccc(-c4ccc(-c5ccc6ccccc6c5)c5oc6ccccc6c45)c4ccccc34)n2)cc1 ; c1ccc(-c2nc(-c3ccccc3)nc(-c3ccc(-c4cccc5oc6c(-c7ccc8ccccc8c7)cccc6c45)c4ccccc34)n2)cc1 ; c1ccc(-c2ccc3oc4cccc(-c5ccc(-c6nc(-c7ccccc7)nc(-c7ccc8ccccc8c7)n6)c6ccccc56)c4c3c2)cc1]

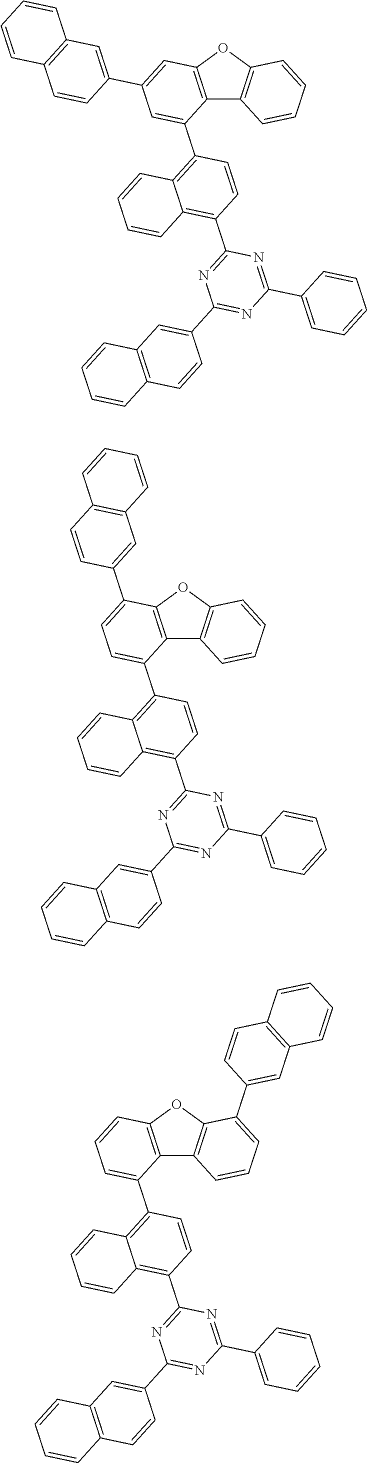

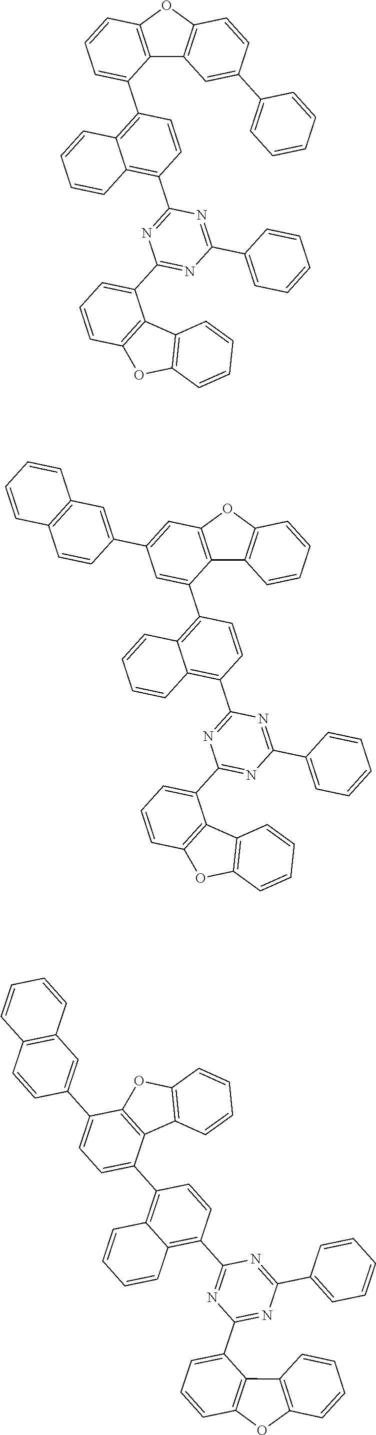 [SMILES: c1ccc(-c2ccc3oc4cccc(-c5ccc(-c6nc(-c7ccccc7)nc(-c7cccc8oc9ccccc9c78)n6)c6ccccc56)c4c3c2)cc1 ; c1ccc(-c2nc(-c3ccc(-c4cc(-c5ccc6ccccc6c5)cc5oc6ccccc6c45)c4ccccc34)nc(-c3cccc4oc5ccccc5c34)n2)cc1 ; c1ccc(-c2nc(-c3ccc(-c4ccc(-c5ccc6ccccc6c5)c5oc6ccccc6c45)c4ccccc34)nc(-c3cccc4oc5ccccc5c34)n2)cc1]

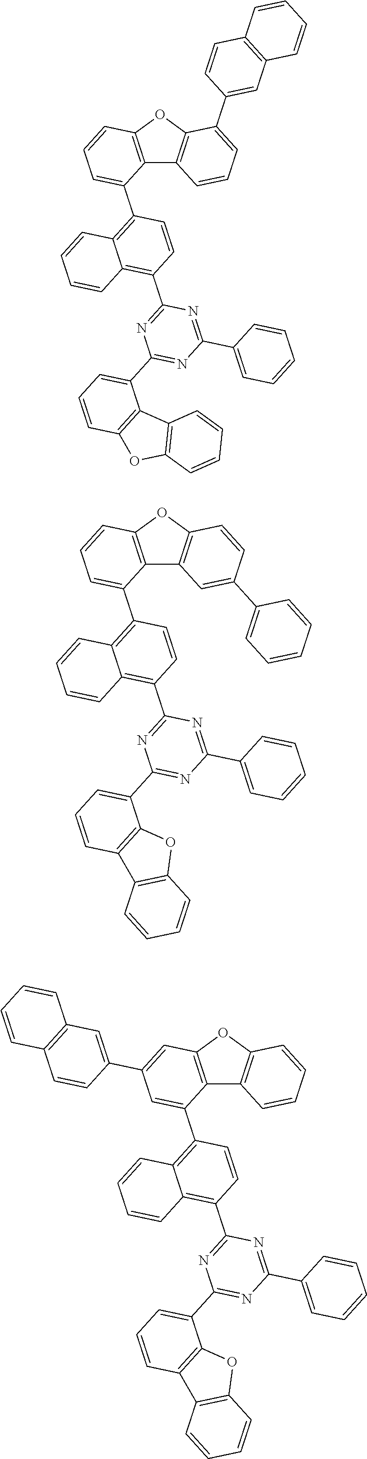 [SMILES: c1ccc(-c2nc(-c3ccc(-c4cccc5oc6c(-c7ccc8ccccc8c7)cccc6c45)c4ccccc34)nc(-c3cccc4oc5ccccc5c34)n2)cc1 ; c1ccc(-c2ccc3oc4cccc(-c5ccc(-c6nc(-c7ccccc7)nc(-c7cccc8c7oc7ccccc78)n6)c6ccccc56)c4c3c2)cc1 ; c1ccc(-c2nc(-c3ccc(-c4cc(-c5ccc6ccccc6c5)cc5oc6ccccc6c45)c4ccccc34)nc(-c3cccc4c3oc3ccccc34)n2)cc1]

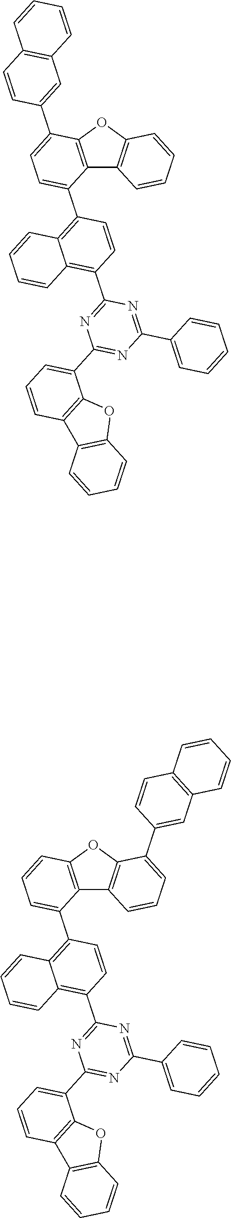 [SMILES: c1ccc(-c2nc(-c3ccc(-c4ccc(-c5ccc6ccccc6c5)c5oc6ccccc6c45)c4ccccc34)nc(-c3cccc4c3oc3ccccc34)n2)cc1 ; c1ccc(-c2nc(-c3ccc(-c4cccc5oc6c(-c7ccc8ccccc8c7)cccc6c45)c4ccccc34)nc(-c3cccc4c3oc3ccccc34)n2)cc1]

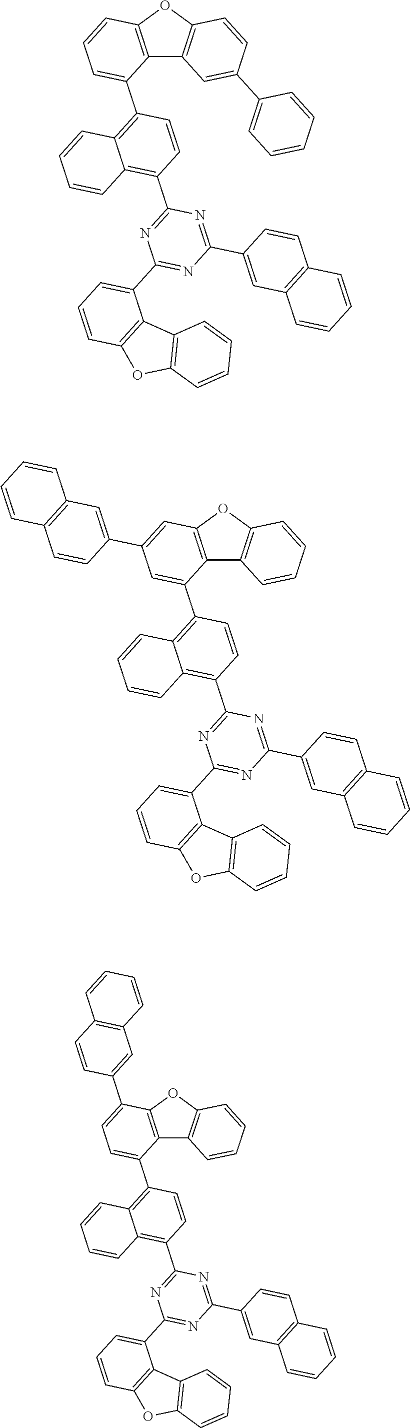 [SMILES: c1ccc(-c2ccc3oc4cccc(-c5ccc(-c6nc(-c7ccc8ccccc8c7)nc(-c7cccc8oc9ccccc9c78)n6)c6ccccc56)c4c3c2)cc1 ; c1ccc2cc(-c3cc(-c4ccc(-c5nc(-c6ccc7ccccc7c6)nc(-c6cccc7oc8ccccc8c67)n5)c5ccccc45)c4c(c3)oc3ccccc34)ccc2c1 ; c1ccc2cc(-c3nc(-c4ccc(-c5ccc(-c6ccc7ccccc7c6)c6oc7ccccc7c56)c5ccccc45)nc(-c4cccc5oc6ccccc6c45)n3)ccc2c1]

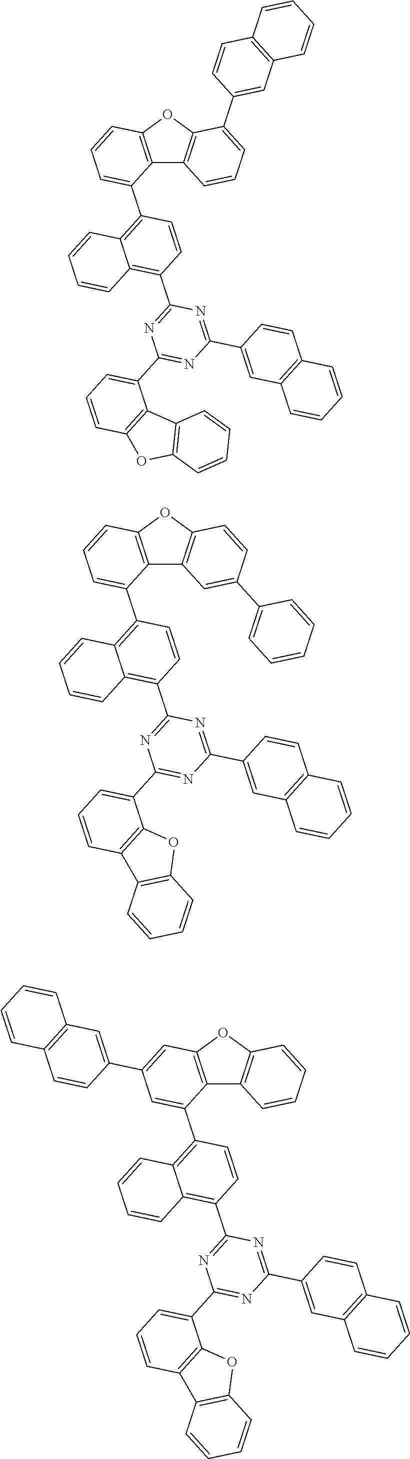 [SMILES: c1ccc2cc(-c3nc(-c4ccc(-c5cccc6oc7c(-c8ccc9ccccc9c8)cccc7c56)c5ccccc45)nc(-c4cccc5oc6ccccc6c45)n3)ccc2c1 ; c1ccc(-c2ccc3oc4cccc(-c5ccc(-c6nc(-c7ccc8ccccc8c7)nc(-c7cccc8c7oc7ccccc78)n6)c6ccccc56)c4c3c2)cc1 ; c1ccc2cc(-c3cc(-c4ccc(-c5nc(-c6ccc7ccccc7c6)nc(-c6cccc7c6oc6ccccc67)n5)c5ccccc45)c4c(c3)oc3ccccc34)ccc2c1]

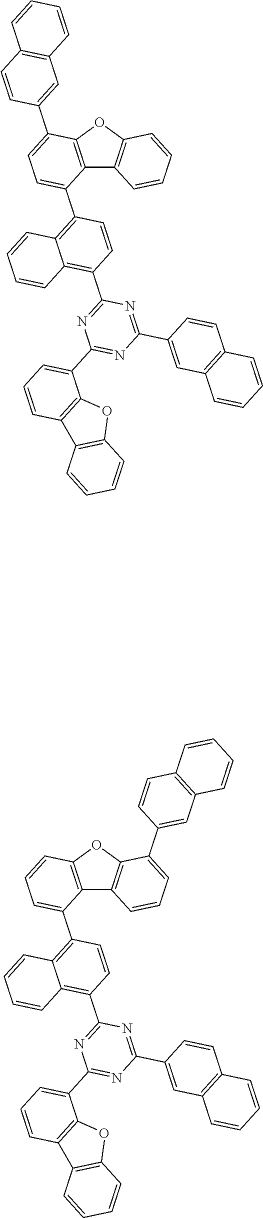 [SMILES: c1ccc2cc(-c3nc(-c4ccc(-c5ccc(-c6ccc7ccccc7c6)c6oc7ccccc7c56)c5ccccc45)nc(-c4cccc5c4oc4ccccc45)n3)ccc2c1 ; c1ccc2cc(-c3nc(-c4ccc(-c5cccc6oc7c(-c8ccc9ccccc9c8)cccc7c56)c5ccccc45)nc(-c4cccc5c4oc4ccccc45)n3)ccc2c1]

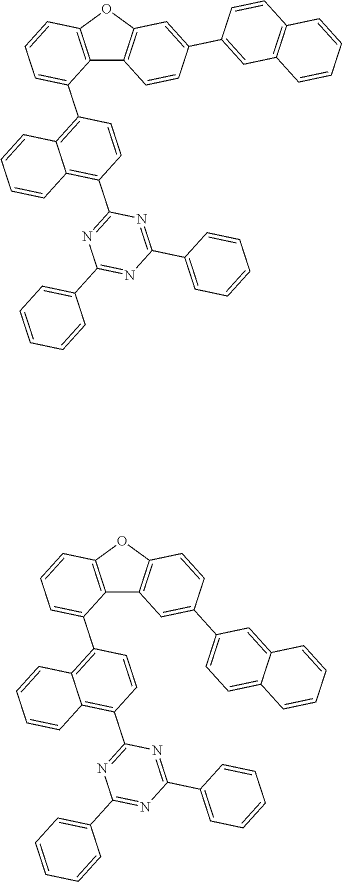 [SMILES: c1ccc(-c2nc(-c3ccccc3)nc(-c3ccc(-c4cccc5oc6cc(-c7ccc8ccccc8c7)ccc6c45)c4ccccc34)n2)cc1 ; c1ccc(-c2nc(-c3ccccc3)nc(-c3ccc(-c4cccc5oc6ccc(-c7ccc8ccccc8c7)cc6c45)c4ccccc34)n2)cc1]

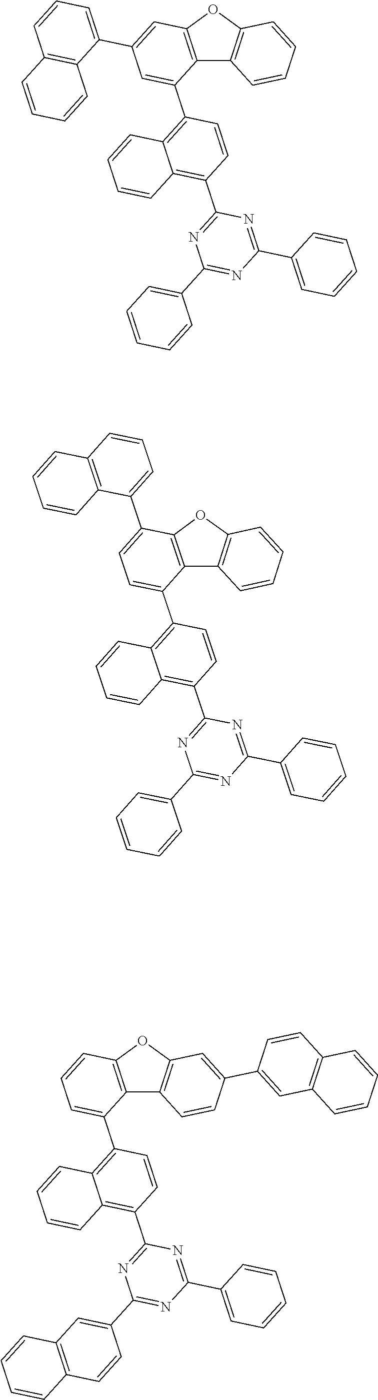 [SMILES: c1ccc(-c2nc(-c3ccccc3)nc(-c3ccc(-c4cc(-c5cccc6ccccc56)cc5oc6ccccc6c45)c4ccccc34)n2)cc1 ; c1ccc(-c2nc(-c3ccccc3)nc(-c3ccc(-c4ccc(-c5cccc6ccccc56)c5oc6ccccc6c45)c4ccccc34)n2)cc1 ; c1ccc(-c2nc(-c3ccc4ccccc4c3)nc(-c3ccc(-c4cccc5oc6cc(-c7ccc8ccccc8c7)ccc6c45)c4ccccc34)n2)cc1]

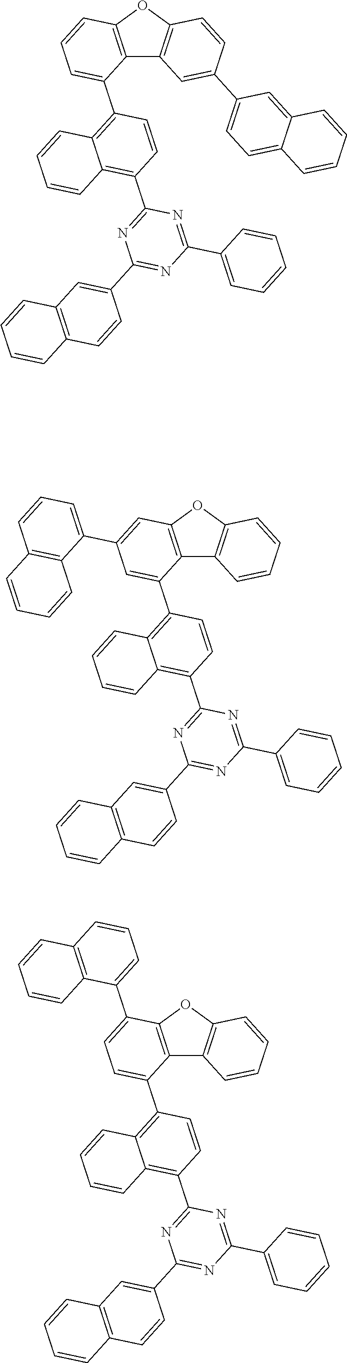 [SMILES: c1ccc(-c2nc(-c3ccc4ccccc4c3)nc(-c3ccc(-c4cccc5oc6ccc(-c7ccc8ccccc8c7)cc6c45)c4ccccc34)n2)cc1 ; c1ccc(-c2nc(-c3ccc4ccccc4c3)nc(-c3ccc(-c4cc(-c5cccc6ccccc56)cc5oc6ccccc6c45)c4ccccc34)n2)cc1 ; c1ccc(-c2nc(-c3ccc4ccccc4c3)nc(-c3ccc(-c4ccc(-c5cccc6ccccc56)c5oc6ccccc6c45)c4ccccc34)n2)cc1]

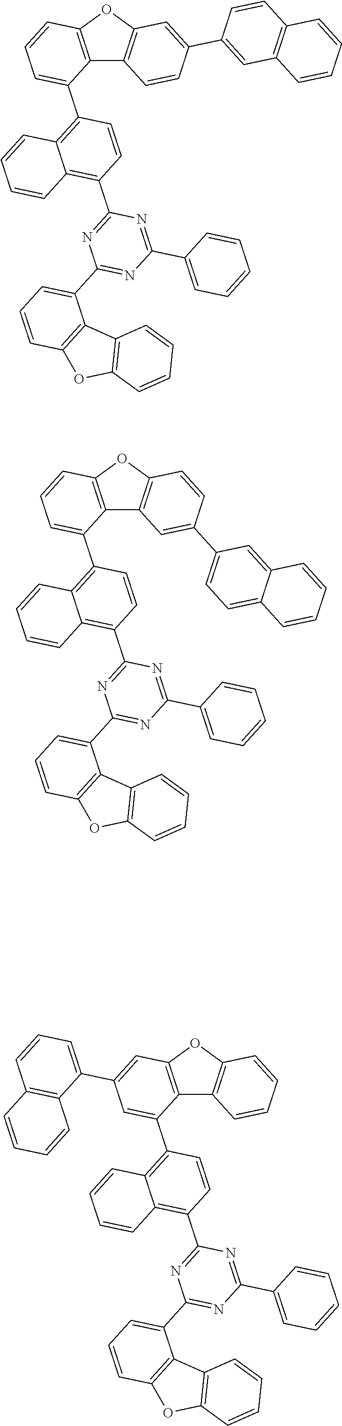 [SMILES: c1ccc(-c2nc(-c3ccc(-c4cccc5oc6cc(-c7ccc8ccccc8c7)ccc6c45)c4ccccc34)nc(-c3cccc4oc5ccccc5c34)n2)cc1 ; c1ccc(-c2nc(-c3ccc(-c4cccc5oc6ccc(-c7ccc8ccccc8c7)cc6c45)c4ccccc34)nc(-c3cccc4oc5ccccc5c34)n2)cc1 ; c1ccc(-c2nc(-c3ccc(-c4cc(-c5cccc6ccccc56)cc5oc6ccccc6c45)c4ccccc34)nc(-c3cccc4oc5ccccc5c34)n2)cc1]

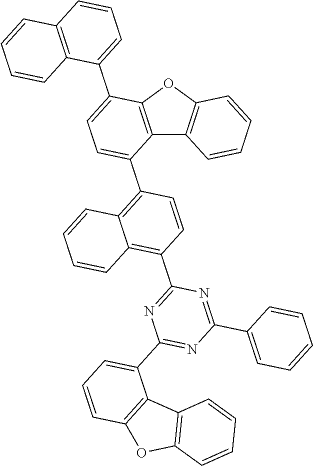 [SMILES: c1ccc(-c2nc(-c3ccc(-c4ccc(-c5cccc6ccccc56)c5oc6ccccc6c45)c4ccccc34)nc(-c3cccc4oc5ccccc5c34)n2)cc1]

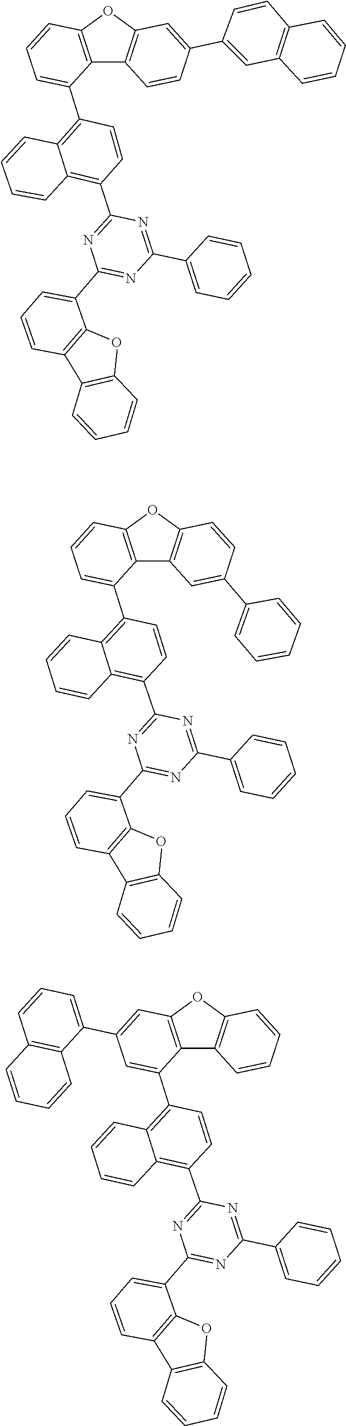 [SMILES: c1ccc(-c2nc(-c3ccc(-c4cccc5oc6cc(-c7ccc8ccccc8c7)ccc6c45)c4ccccc34)nc(-c3cccc4c3oc3ccccc34)n2)cc1 ; c1ccc(-c2ccc3oc4cccc(-c5ccc(-c6nc(-c7ccccc7)nc(-c7cccc8c7oc7ccccc78)n6)c6ccccc56)c4c3c2)cc1 ; c1ccc(-c2nc(-c3ccc(-c4cc(-c5cccc6ccccc56)cc5oc6ccccc6c45)c4ccccc34)nc(-c3cccc4c3oc3ccccc34)n2)cc1]

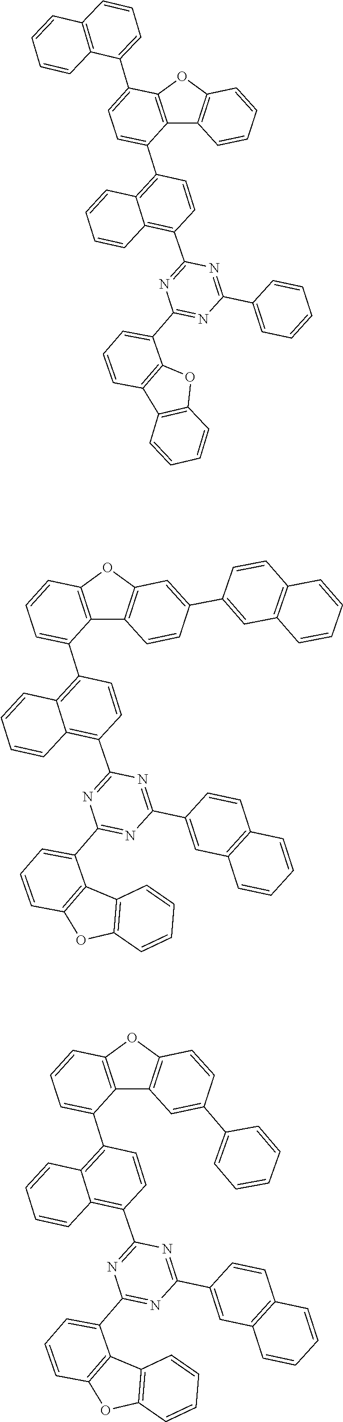 [SMILES: c1ccc(-c2nc(-c3ccc(-c4ccc(-c5cccc6ccccc56)c5oc6ccccc6c45)c4ccccc34)nc(-c3cccc4c3oc3ccccc34)n2)cc1 ; c1ccc2cc(-c3ccc4c(c3)oc3cccc(-c5ccc(-c6nc(-c7ccc8ccccc8c7)nc(-c7cccc8oc9ccccc9c78)n6)c6ccccc56)c34)ccc2c1 ; c1ccc(-c2ccc3oc4cccc(-c5ccc(-c6nc(-c7ccc8ccccc8c7)nc(-c7cccc8oc9ccccc9c78)n6)c6ccccc56)c4c3c2)cc1]

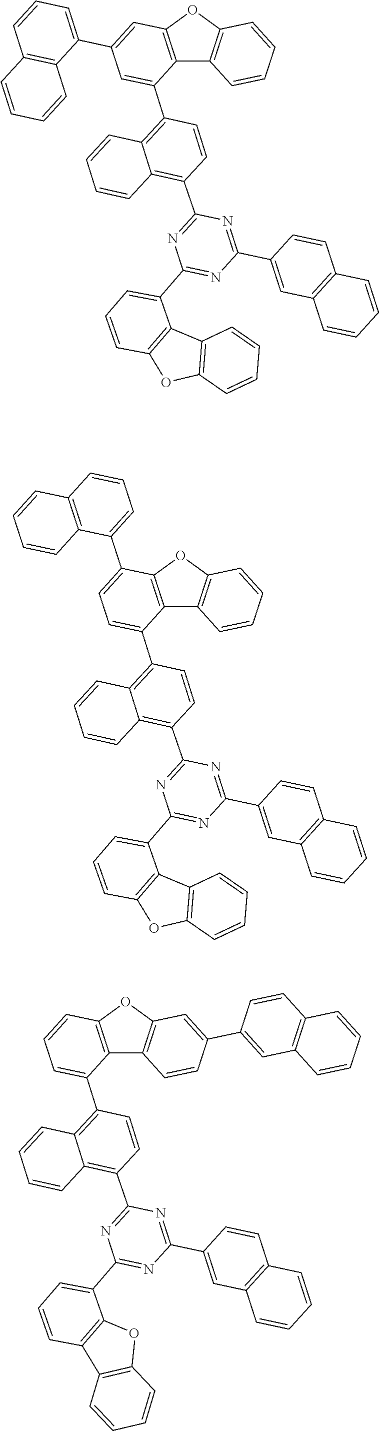 [SMILES: c1ccc2cc(-c3nc(-c4ccc(-c5cc(-c6cccc7ccccc67)cc6oc7ccccc7c56)c5ccccc45)nc(-c4cccc5oc6ccccc6c45)n3)ccc2c1 ; c1ccc2cc(-c3nc(-c4ccc(-c5ccc(-c6cccc7ccccc67)c6oc7ccccc7c56)c5ccccc45)nc(-c4cccc5oc6ccccc6c45)n3)ccc2c1 ; c1ccc2cc(-c3ccc4c(c3)oc3cccc(-c5ccc(-c6nc(-c7ccc8ccccc8c7)nc(-c7cccc8c7oc7ccccc78)n6)c6ccccc56)c34)ccc2c1]

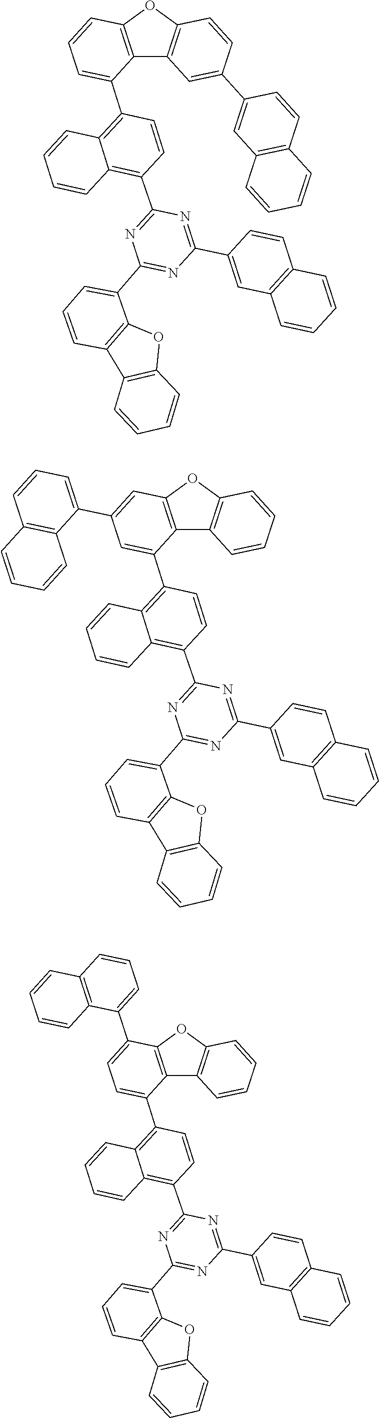 [SMILES: c1ccc2cc(-c3ccc4oc5cccc(-c6ccc(-c7nc(-c8ccc9ccccc9c8)nc(-c8cccc9c8oc8ccccc89)n7)c7ccccc67)c5c4c3)ccc2c1 ; c1ccc2cc(-c3nc(-c4ccc(-c5cc(-c6cccc7ccccc67)cc6oc7ccccc7c56)c5ccccc45)nc(-c4cccc5c4oc4ccccc45)n3)ccc2c1 ; c1ccc2cc(-c3nc(-c4ccc(-c5ccc(-c6cccc7ccccc67)c6oc7ccccc7c56)c5ccccc45)nc(-c4cccc5c4oc4ccccc45)n3)ccc2c1]

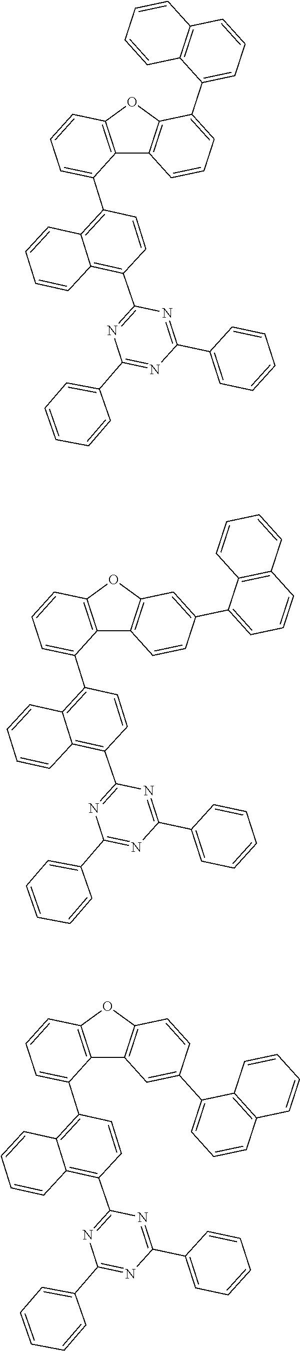 [SMILES: c1ccc(-c2nc(-c3ccccc3)nc(-c3ccc(-c4cccc5oc6c(-c7cccc8ccccc78)cccc6c45)c4ccccc34)n2)cc1 ; c1ccc(-c2nc(-c3ccccc3)nc(-c3ccc(-c4cccc5oc6cc(-c7cccc8ccccc78)ccc6c45)c4ccccc34)n2)cc1 ; c1ccc(-c2nc(-c3ccccc3)nc(-c3ccc(-c4cccc5oc6ccc(-c7cccc8ccccc78)cc6c45)c4ccccc34)n2)cc1]

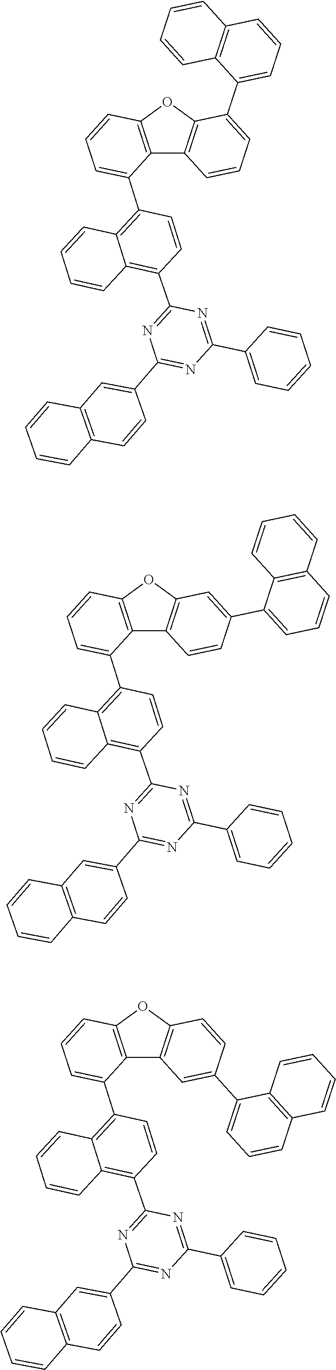 [SMILES: c1ccc(-c2nc(-c3ccc4ccccc4c3)nc(-c3ccc(-c4cccc5oc6c(-c7cccc8ccccc78)cccc6c45)c4ccccc34)n2)cc1 ; c1ccc(-c2nc(-c3ccc4ccccc4c3)nc(-c3ccc(-c4cccc5oc6cc(-c7cccc8ccccc78)ccc6c45)c4ccccc34)n2)cc1 ; c1ccc(-c2nc(-c3ccc4ccccc4c3)nc(-c3ccc(-c4cccc5oc6ccc(-c7cccc8ccccc78)cc6c45)c4ccccc34)n2)cc1]

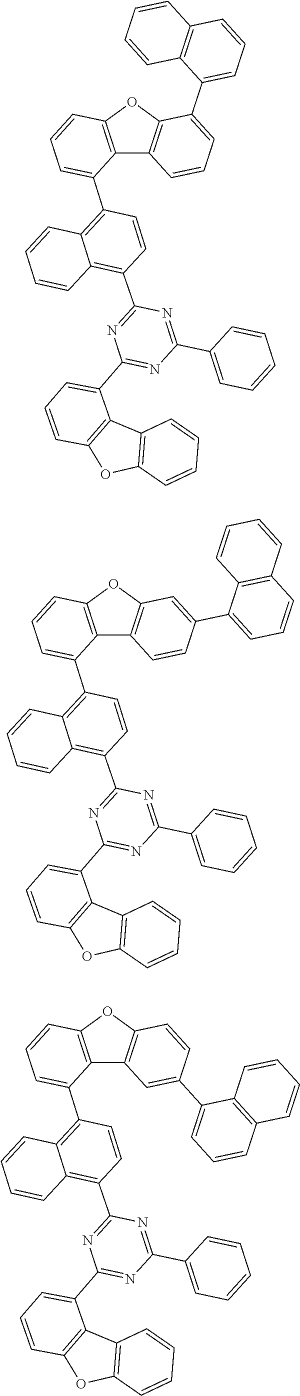 [SMILES: c1ccc(-c2nc(-c3ccc(-c4cccc5oc6c(-c7cccc8ccccc78)cccc6c45)c4ccccc34)nc(-c3cccc4oc5ccccc5c34)n2)cc1 ; c1ccc(-c2nc(-c3ccc(-c4cccc5oc6cc(-c7cccc8ccccc78)ccc6c45)c4ccccc34)nc(-c3cccc4oc5ccccc5c34)n2)cc1 ; c1ccc(-c2nc(-c3ccc(-c4cccc5oc6ccc(-c7cccc8ccccc78)cc6c45)c4ccccc34)nc(-c3cccc4oc5ccccc5c34)n2)cc1]

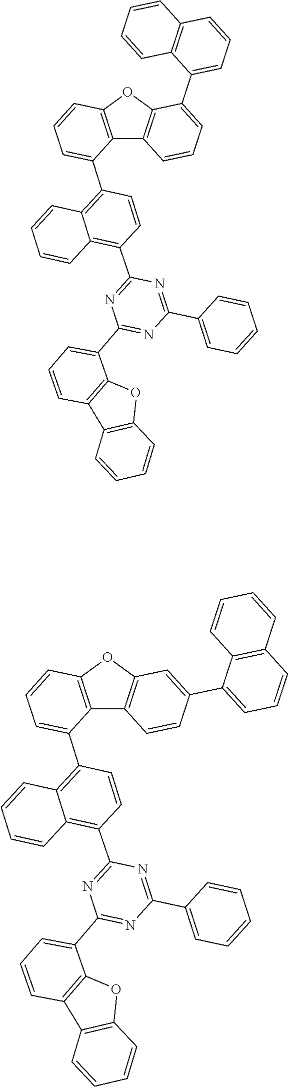 [SMILES: c1ccc(-c2nc(-c3ccc(-c4cccc5oc6c(-c7cccc8ccccc78)cccc6c45)c4ccccc34)nc(-c3cccc4c3oc3ccccc34)n2)cc1 ; c1ccc(-c2nc(-c3ccc(-c4cccc5oc6cc(-c7cccc8ccccc78)ccc6c45)c4ccccc34)nc(-c3cccc4c3oc3ccccc34)n2)cc1]

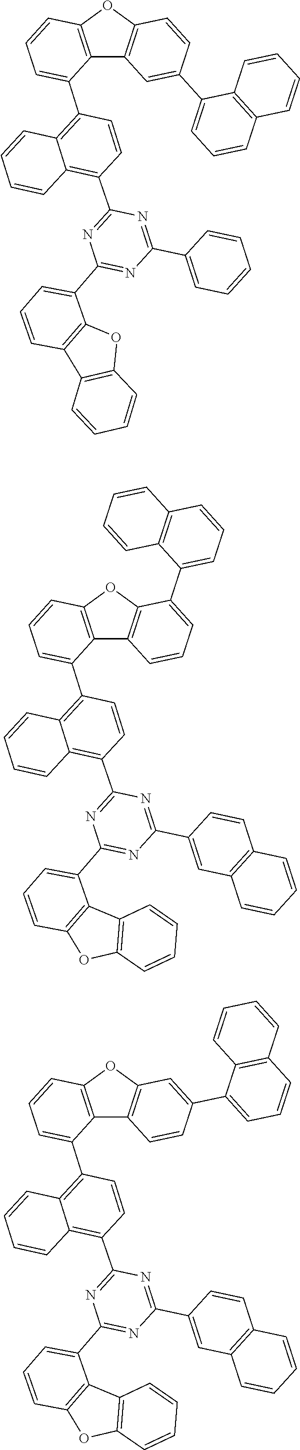 [SMILES: c1ccc(-c2nc(-c3ccc(-c4cccc5oc6ccc(-c7cccc8ccccc78)cc6c45)c4ccccc34)nc(-c3cccc4c3oc3ccccc34)n2)cc1 ; c1ccc2cc(-c3nc(-c4ccc(-c5cccc6oc7c(-c8cccc9ccccc89)cccc7c56)c5ccccc45)nc(-c4cccc5oc6ccccc6c45)n3)ccc2c1 ; c1ccc2cc(-c3nc(-c4ccc(-c5cccc6oc7cc(-c8cccc9ccccc89)ccc7c56)c5ccccc45)nc(-c4cccc5oc6ccccc6c45)n3)ccc2c1]

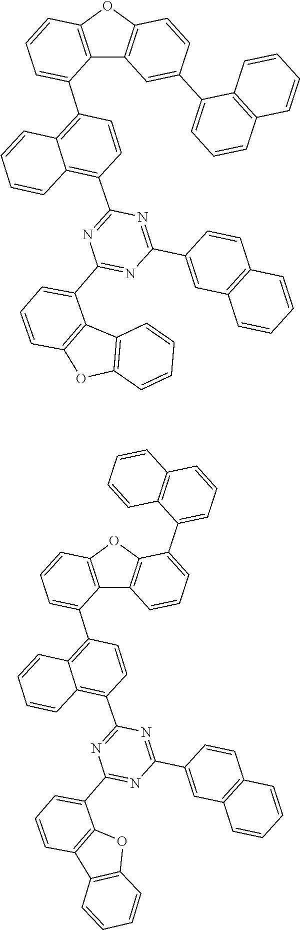 [SMILES: c1ccc2cc(-c3nc(-c4ccc(-c5cccc6oc7ccc(-c8cccc9ccccc89)cc7c56)c5ccccc45)nc(-c4cccc5oc6ccccc6c45)n3)ccc2c1 ; c1ccc2cc(-c3nc(-c4ccc(-c5cccc6oc7c(-c8cccc9ccccc89)cccc7c56)c5ccccc45)nc(-c4cccc5c4oc4ccccc45)n3)ccc2c1]

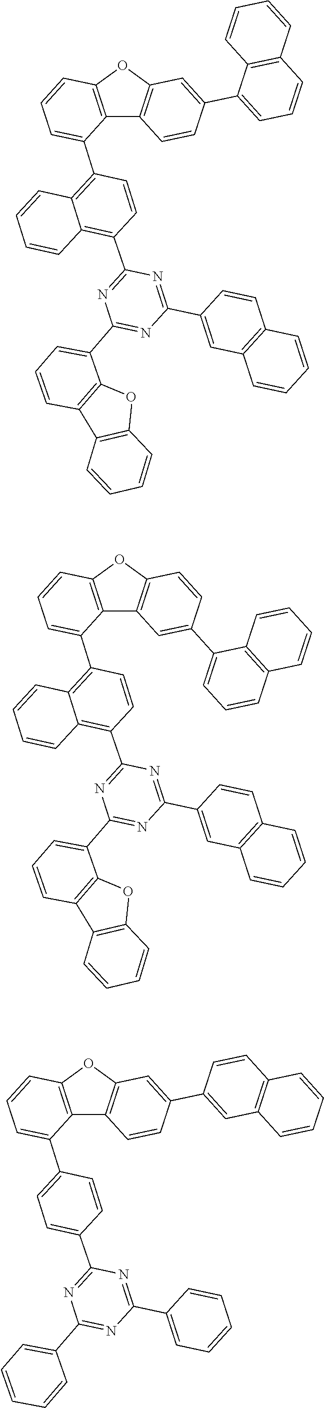 [SMILES: c1ccc2cc(-c3nc(-c4ccc(-c5cccc6oc7cc(-c8cccc9ccccc89)ccc7c56)c5ccccc45)nc(-c4cccc5c4oc4ccccc45)n3)ccc2c1 ; c1ccc2cc(-c3nc(-c4ccc(-c5cccc6oc7ccc(-c8cccc9ccccc89)cc7c56)c5ccccc45)nc(-c4cccc5c4oc4ccccc45)n3)ccc2c1 ; c1ccc(-c2nc(-c3ccccc3)nc(-c3ccc(-c4cccc5oc6cc(-c7ccc8ccccc8c7)ccc6c45)cc3)n2)cc1]

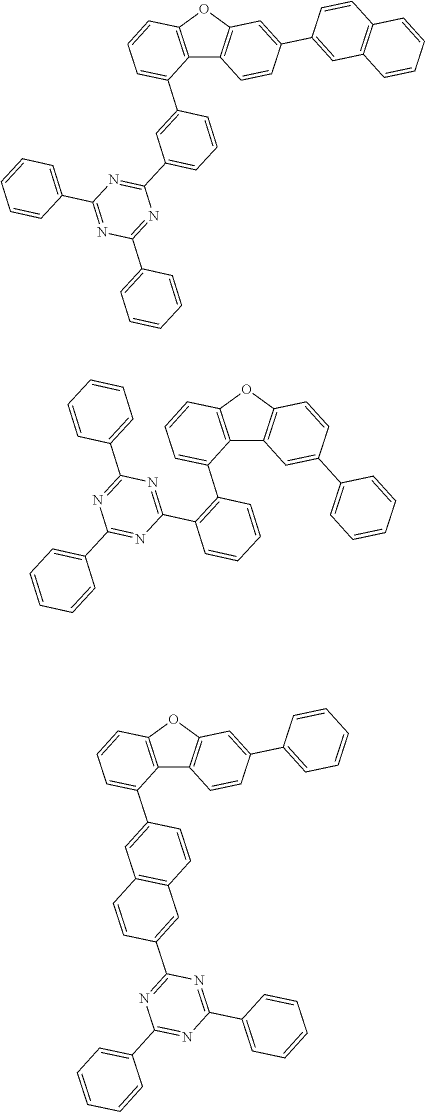 [SMILES: c1ccc(-c2nc(-c3ccccc3)nc(-c3cccc(-c4cccc5oc6cc(-c7ccc8ccccc8c7)ccc6c45)c3)n2)cc1 ; c1ccc(-c2ccc3oc4cccc(-c5ccccc5-c5nc(-c6ccccc6)nc(-c6ccccc6)n5)c4c3c2)cc1 ; c1ccc(-c2ccc3c(c2)oc2cccc(-c4ccc5cc(-c6nc(-c7ccccc7)nc(-c7ccccc7)n6)ccc5c4)c23)cc1]

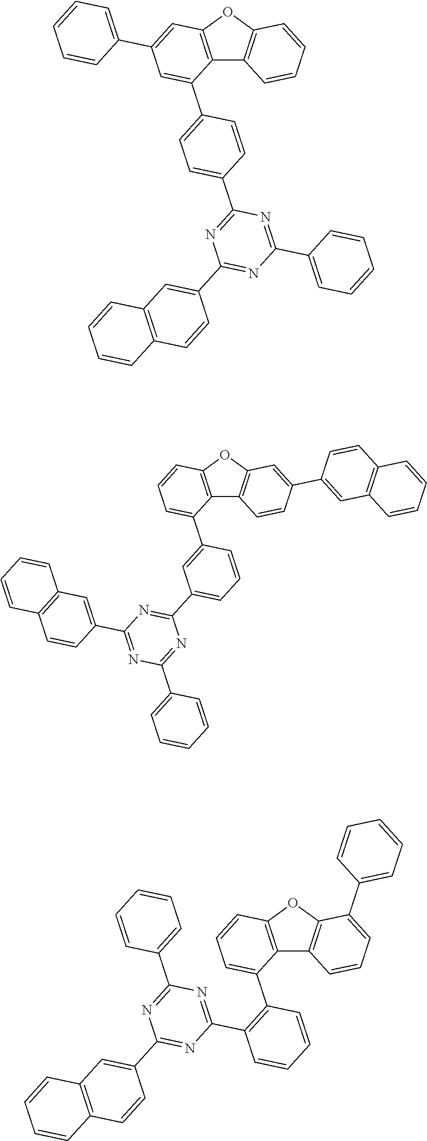 [SMILES: c1ccc(-c2cc(-c3ccc(-c4nc(-c5ccccc5)nc(-c5ccc6ccccc6c5)n4)cc3)c3c(c2)oc2ccccc23)cc1 ; c1ccc(-c2nc(-c3cccc(-c4cccc5oc6cc(-c7ccc8ccccc8c7)ccc6c45)c3)nc(-c3ccc4ccccc4c3)n2)cc1 ; c1ccc(-c2nc(-c3ccc4ccccc4c3)nc(-c3ccccc3-c3cccc4oc5c(-c6ccccc6)cccc5c34)n2)cc1]

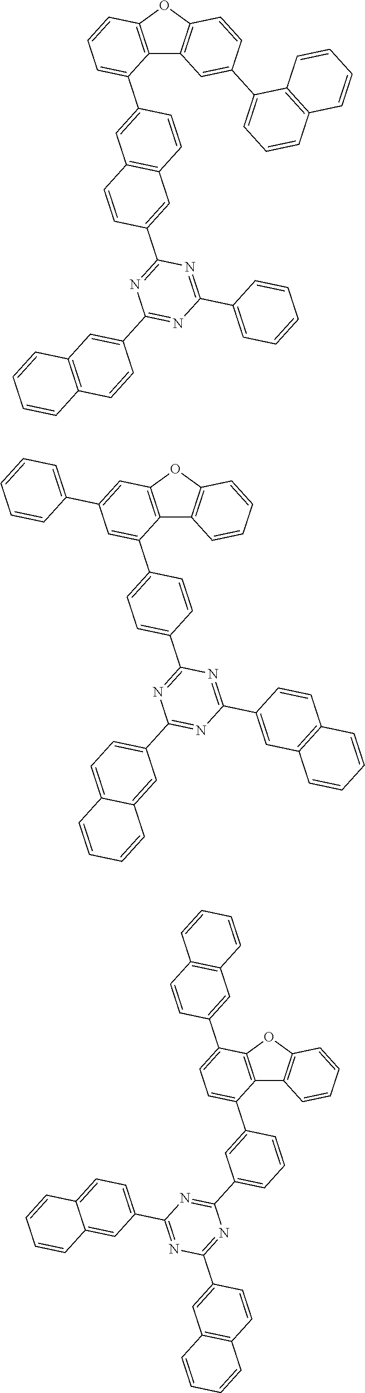 [SMILES: c1ccc(-c2nc(-c3ccc4ccccc4c3)nc(-c3ccc4cc(-c5cccc6oc7ccc(-c8cccc9ccccc89)cc7c56)ccc4c3)n2)cc1 ; c1ccc(-c2cc(-c3ccc(-c4nc(-c5ccc6ccccc6c5)nc(-c5ccc6ccccc6c5)n4)cc3)c3c(c2)oc2ccccc23)cc1 ; c1cc(-c2nc(-c3ccc4ccccc4c3)nc(-c3ccc4ccccc4c3)n2)cc(-c2ccc(-c3ccc4ccccc4c3)c3oc4ccccc4c23)c1]

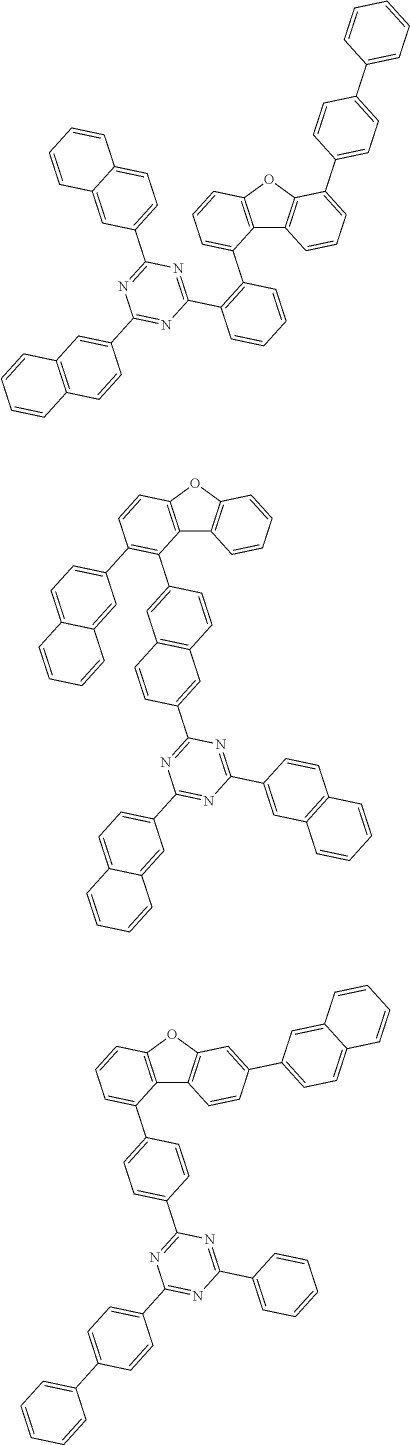 [SMILES: c1ccc(-c2ccc(-c3cccc4c3oc3cccc(-c5ccccc5-c5nc(-c6ccc7ccccc7c6)nc(-c6ccc7ccccc7c6)n5)c34)cc2)cc1 ; c1ccc2cc(-c3nc(-c4ccc5ccccc5c4)nc(-c4ccc5cc(-c6c(-c7ccc8ccccc8c7)ccc7oc8ccccc8c67)ccc5c4)n3)ccc2c1 ; c1ccc(-c2ccc(-c3nc(-c4ccccc4)nc(-c4ccc(-c5cccc6oc7cc(-c8ccc9ccccc9c8)ccc7c56)cc4)n3)cc2)cc1]

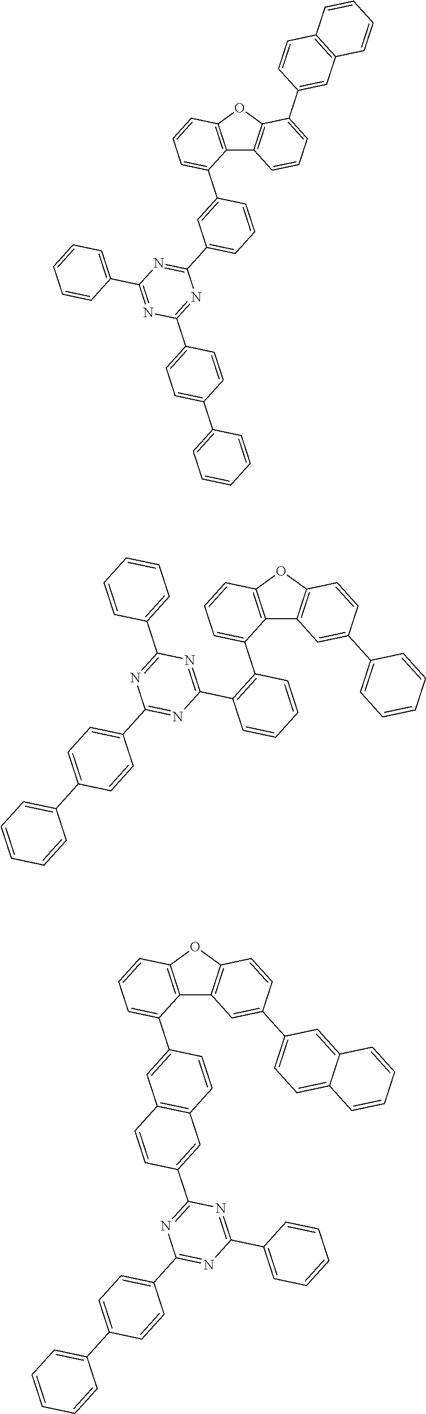 [SMILES: c1ccc(-c2ccc(-c3nc(-c4ccccc4)nc(-c4cccc(-c5cccc6oc7c(-c8ccc9ccccc9c8)cccc7c56)c4)n3)cc2)cc1 ; c1ccc(-c2ccc(-c3nc(-c4ccccc4)nc(-c4ccccc4-c4cccc5oc6ccc(-c7ccccc7)cc6c45)n3)cc2)cc1 ; c1ccc(-c2ccc(-c3nc(-c4ccccc4)nc(-c4ccc5cc(-c6cccc7oc8ccc(-c9ccc%10ccccc%10c9)cc8c67)ccc5c4)n3)cc2)cc1]

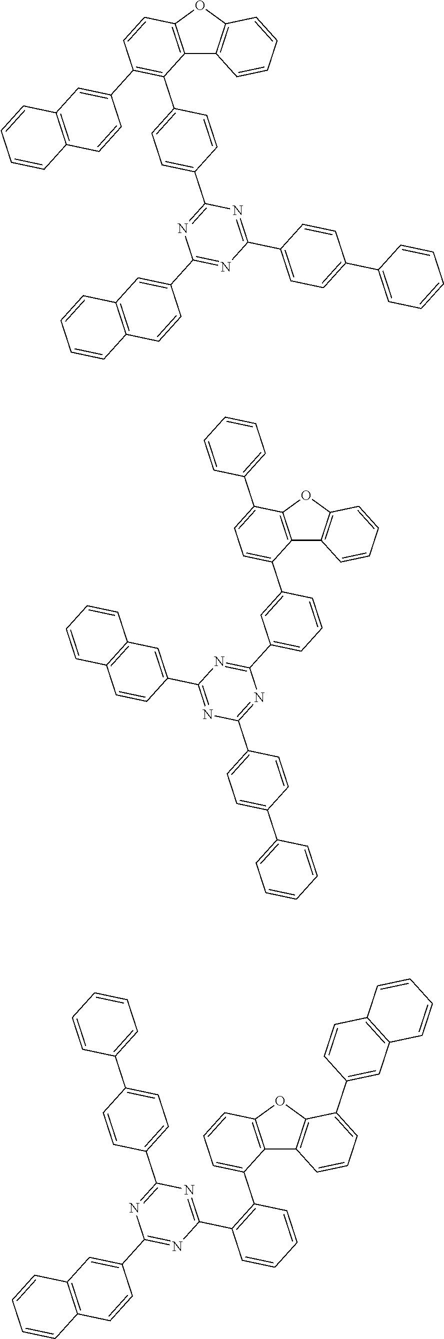 [SMILES: c1ccc(-c2ccc(-c3nc(-c4ccc(-c5c(-c6ccc7ccccc7c6)ccc6oc7ccccc7c56)cc4)nc(-c4ccc5ccccc5c4)n3)cc2)cc1 ; c1ccc(-c2ccc(-c3nc(-c4cccc(-c5ccc(-c6ccccc6)c6oc7ccccc7c56)c4)nc(-c4ccc5ccccc5c4)n3)cc2)cc1 ; c1ccc(-c2ccc(-c3nc(-c4ccc5ccccc5c4)nc(-c4ccccc4-c4cccc5oc6c(-c7ccc8ccccc8c7)cccc6c45)n3)cc2)cc1]

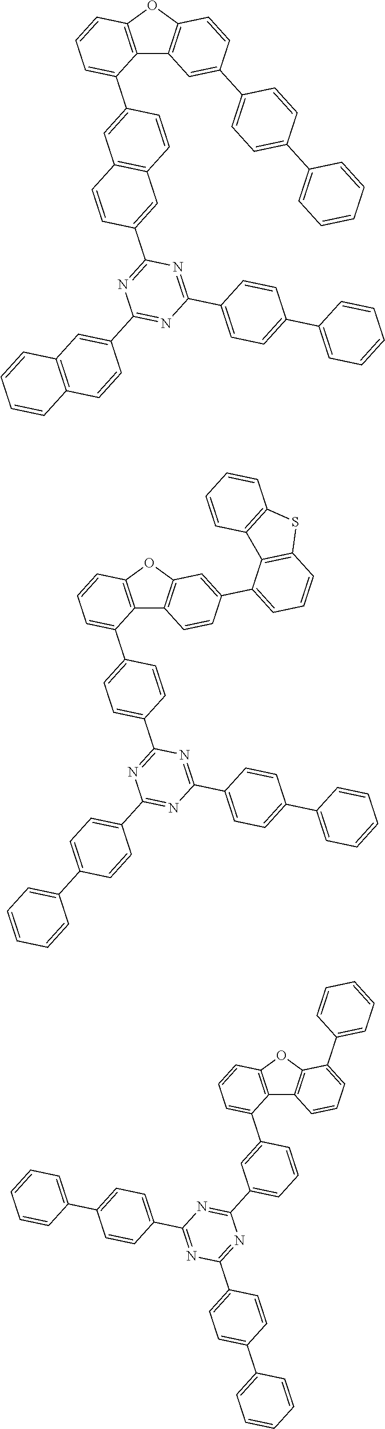 [SMILES: c1ccc(-c2ccc(-c3ccc4oc5cccc(-c6ccc7cc(-c8nc(-c9ccc(-c%10ccccc%10)cc9)nc(-c9ccc%10ccccc%10c9)n8)ccc7c6)c5c4c3)cc2)cc1 ; c1ccc(-c2ccc(-c3nc(-c4ccc(-c5ccccc5)cc4)nc(-c4ccc(-c5cccc6oc7cc(-c8cccc9sc%10ccccc%10c89)ccc7c56)cc4)n3)cc2)cc1 ; c1ccc(-c2ccc(-c3nc(-c4ccc(-c5ccccc5)cc4)nc(-c4cccc(-c5cccc6oc7c(-c8ccccc8)cccc7c56)c4)n3)cc2)cc1]

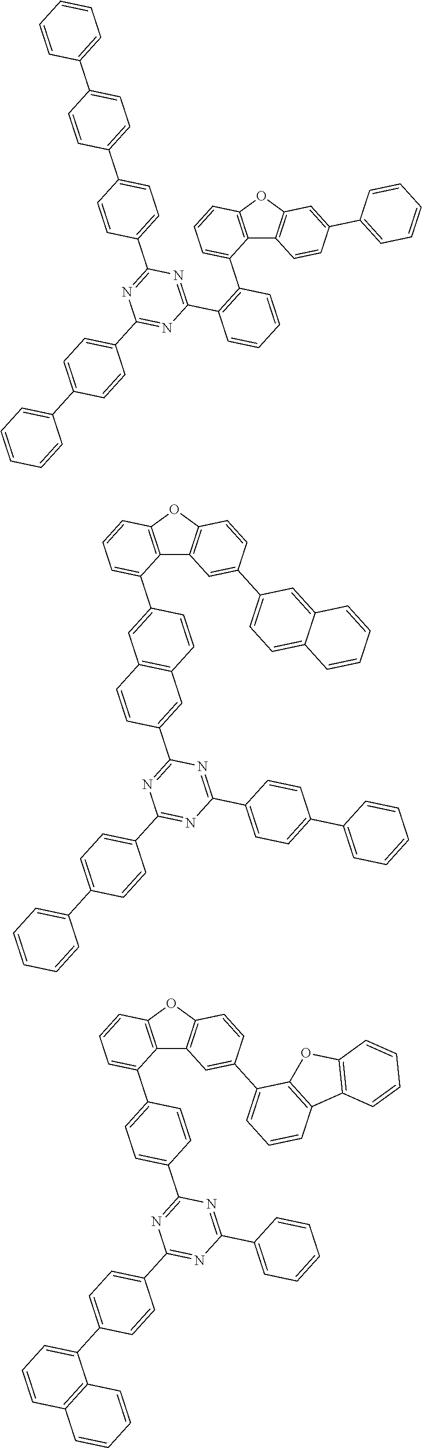 [SMILES: c1ccc(-c2ccc(-c3ccc(-c4nc(-c5ccc(-c6ccccc6)cc5)nc(-c5ccccc5-c5cccc6oc7cc(-c8ccccc8)ccc7c56)n4)cc3)cc2)cc1 ; c1ccc(-c2ccc(-c3nc(-c4ccc(-c5ccccc5)cc4)nc(-c4ccc5cc(-c6cccc7oc8ccc(-c9ccc%10ccccc%10c9)cc8c67)ccc5c4)n3)cc2)cc1 ; c1ccc(-c2nc(-c3ccc(-c4cccc5ccccc45)cc3)nc(-c3ccc(-c4cccc5oc6ccc(-c7cccc8c7oc7ccccc78)cc6c45)cc3)n2)cc1]

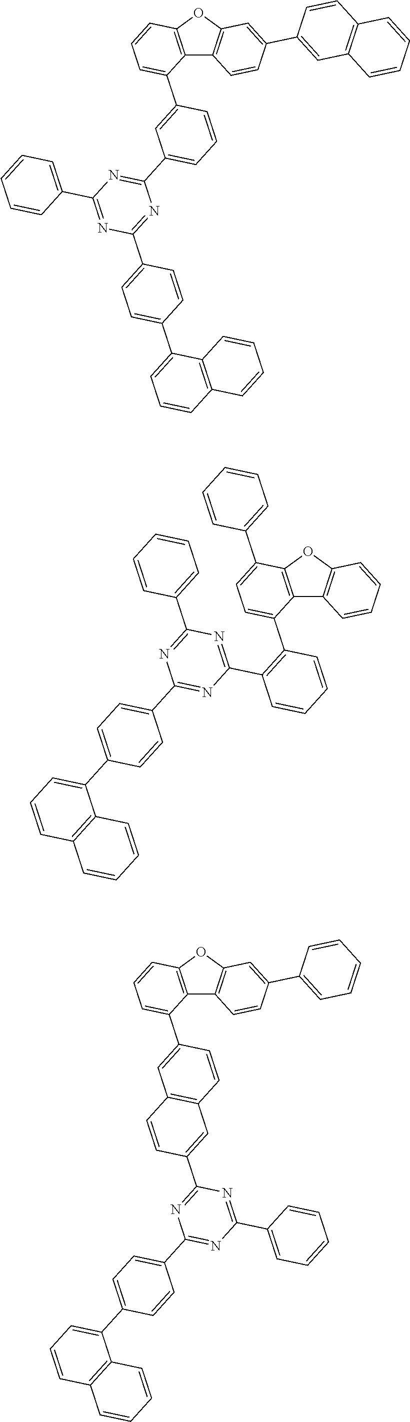 [SMILES: c1ccc(-c2nc(-c3ccc(-c4cccc5ccccc45)cc3)nc(-c3cccc(-c4cccc5oc6cc(-c7ccc8ccccc8c7)ccc6c45)c3)n2)cc1 ; c1ccc(-c2nc(-c3ccc(-c4cccc5ccccc45)cc3)nc(-c3ccccc3-c3ccc(-c4ccccc4)c4oc5ccccc5c34)n2)cc1 ; c1ccc(-c2ccc3c(c2)oc2cccc(-c4ccc5cc(-c6nc(-c7ccccc7)nc(-c7ccc(-c8cccc9ccccc89)cc7)n6)ccc5c4)c23)cc1]

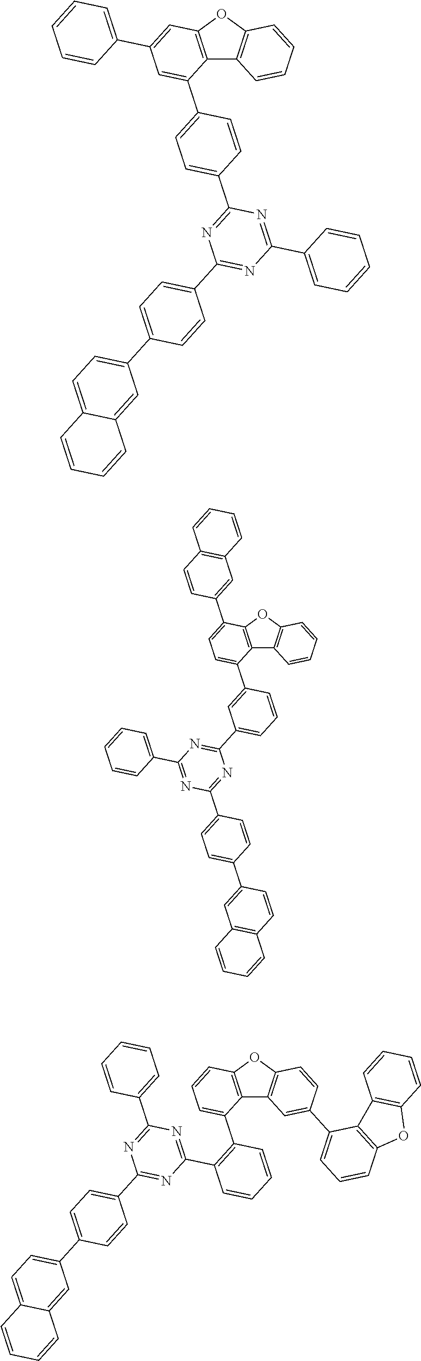 [SMILES: c1ccc(-c2cc(-c3ccc(-c4nc(-c5ccccc5)nc(-c5ccc(-c6ccc7ccccc7c6)cc5)n4)cc3)c3c(c2)oc2ccccc23)cc1 ; c1ccc(-c2nc(-c3ccc(-c4ccc5ccccc5c4)cc3)nc(-c3cccc(-c4ccc(-c5ccc6ccccc6c5)c5oc6ccccc6c45)c3)n2)cc1 ; c1ccc(-c2nc(-c3ccc(-c4ccc5ccccc5c4)cc3)nc(-c3ccccc3-c3cccc4oc5ccc(-c6cccc7oc8ccccc8c67)cc5c34)n2)cc1]

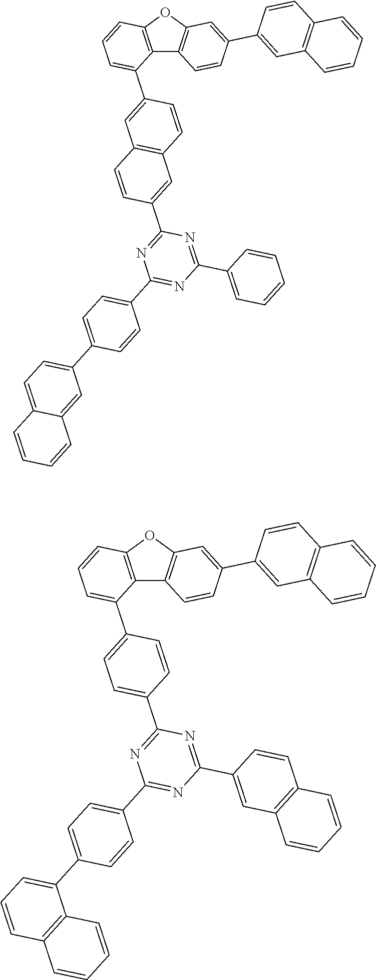 [SMILES: c1ccc(-c2nc(-c3ccc(-c4ccc5ccccc5c4)cc3)nc(-c3ccc4cc(-c5cccc6oc7cc(-c8ccc9ccccc9c8)ccc7c56)ccc4c3)n2)cc1 ; c1ccc2cc(-c3ccc4c(c3)oc3cccc(-c5ccc(-c6nc(-c7ccc(-c8cccc9ccccc89)cc7)nc(-c7ccc8ccccc8c7)n6)cc5)c34)ccc2c1]

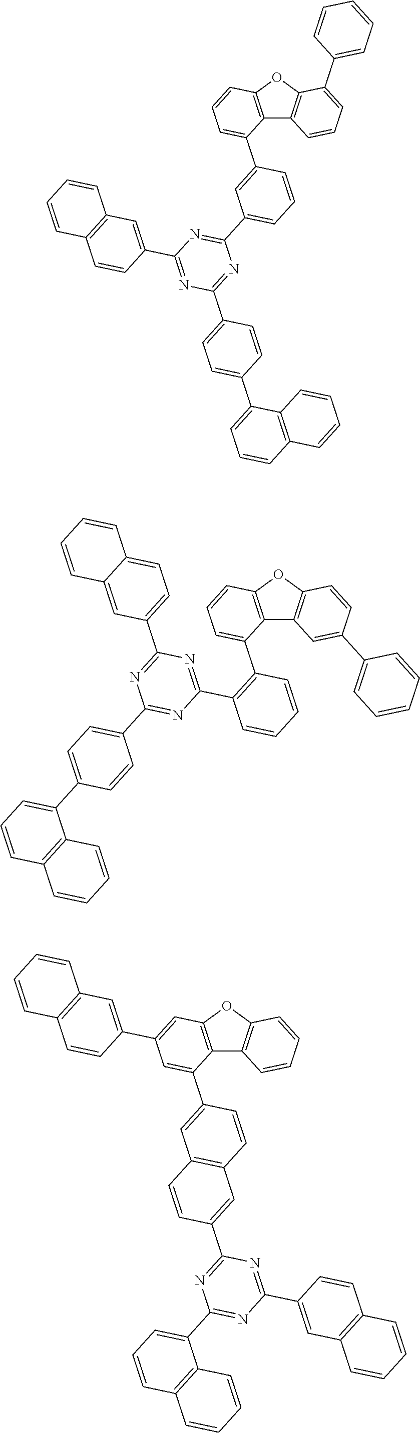 [SMILES: c1ccc(-c2cccc3c2oc2cccc(-c4cccc(-c5nc(-c6ccc(-c7cccc8ccccc78)cc6)nc(-c6ccc7ccccc7c6)n5)c4)c23)cc1 ; c1ccc(-c2ccc3oc4cccc(-c5ccccc5-c5nc(-c6ccc(-c7cccc8ccccc78)cc6)nc(-c6ccc7ccccc7c6)n5)c4c3c2)cc1 ; c1ccc2cc(-c3cc(-c4ccc5cc(-c6nc(-c7ccc8ccccc8c7)nc(-c7cccc8ccccc78)n6)ccc5c4)c4c(c3)oc3ccccc34)ccc2c1]

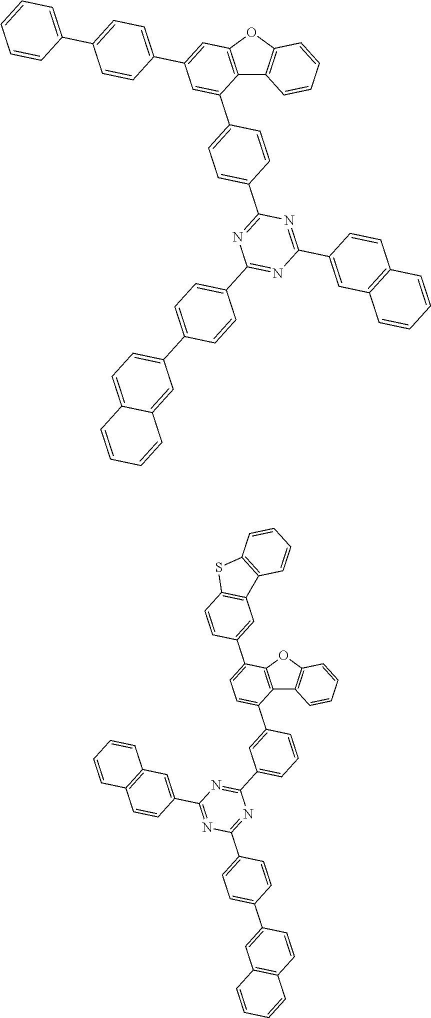 [SMILES: c1ccc(-c2ccc(-c3cc(-c4ccc(-c5nc(-c6ccc(-c7ccc8ccccc8c7)cc6)nc(-c6ccc7ccccc7c6)n5)cc4)c4c(c3)oc3ccccc34)cc2)cc1 ; c1cc(-c2nc(-c3ccc(-c4ccc5ccccc5c4)cc3)nc(-c3ccc4ccccc4c3)n2)cc(-c2ccc(-c3ccc4sc5ccccc5c4c3)c3oc4ccccc4c23)c1]

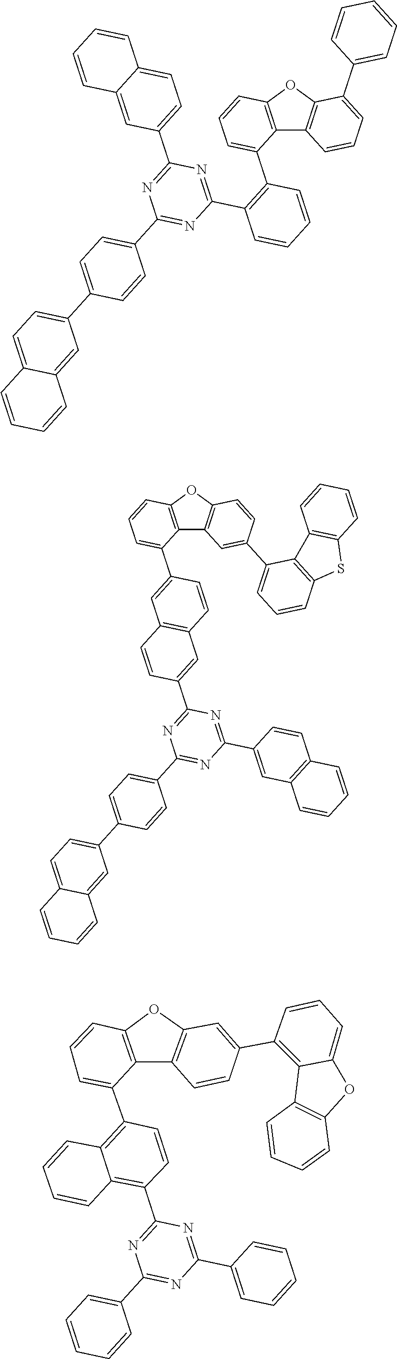 [SMILES: c1ccc(-c2cccc3c2oc2cccc(-c4ccccc4-c4nc(-c5ccc(-c6ccc7ccccc7c6)cc5)nc(-c5ccc6ccccc6c5)n4)c23)cc1 ; c1ccc2cc(-c3ccc(-c4nc(-c5ccc6ccccc6c5)nc(-c5ccc6cc(-c7cccc8oc9ccc(-c%10cccc%11sc%12ccccc%12c%10%11)cc9c78)ccc6c5)n4)cc3)ccc2c1 ; c1ccc(-c2nc(-c3ccccc3)nc(-c3ccc(-c4cccc5oc6cc(-c7cccc8oc9ccccc9c78)ccc6c45)c4ccccc34)n2)cc1]

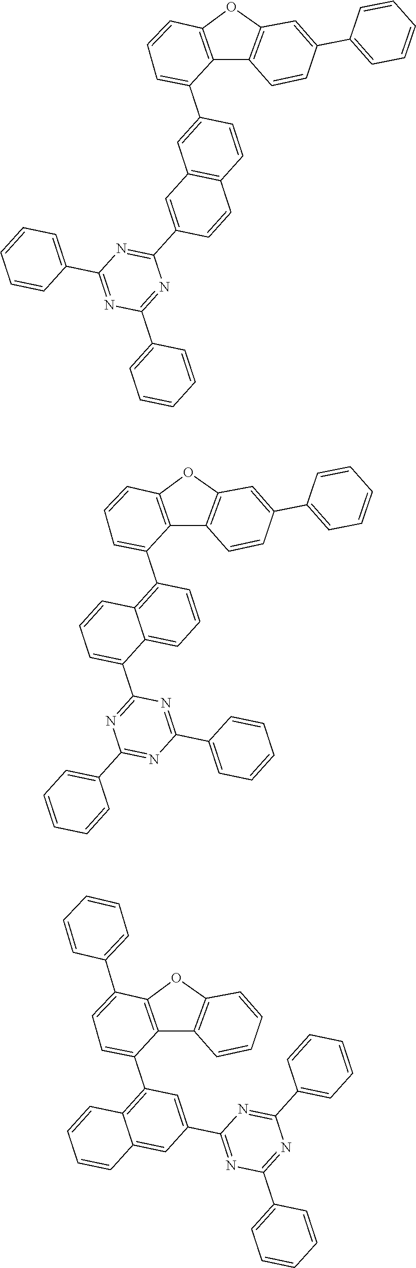 [SMILES: c1ccc(-c2ccc3c(c2)oc2cccc(-c4ccc5ccc(-c6nc(-c7ccccc7)nc(-c7ccccc7)n6)cc5c4)c23)cc1 ; c1ccc(-c2ccc3c(c2)oc2cccc(-c4cccc5c(-c6nc(-c7ccccc7)nc(-c7ccccc7)n6)cccc45)c23)cc1 ; c1ccc(-c2nc(-c3ccccc3)nc(-c3cc(-c4ccc(-c5ccccc5)c5oc6ccccc6c45)c4ccccc4c3)n2)cc1]

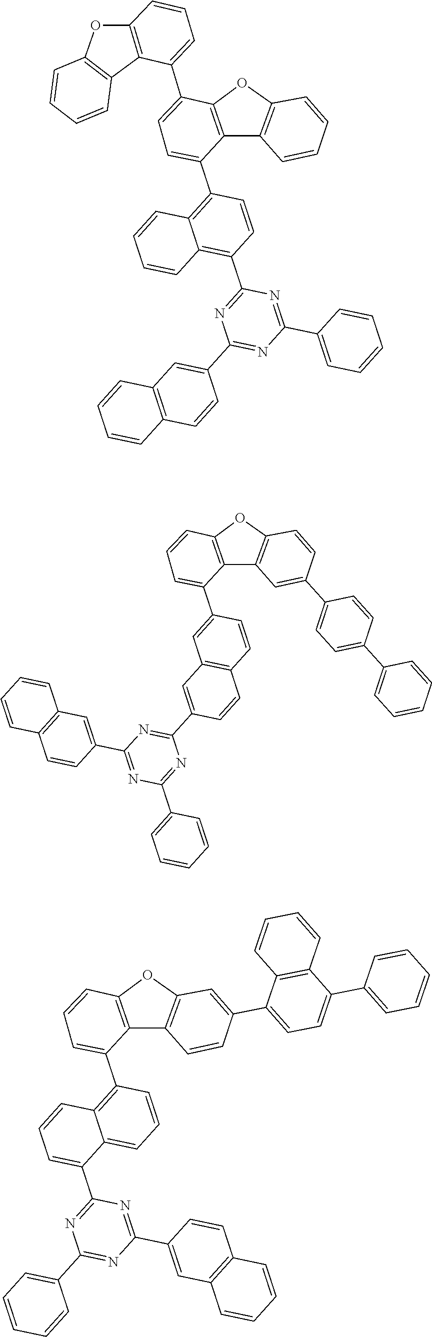 [SMILES: c1ccc(-c2nc(-c3ccc4ccccc4c3)nc(-c3ccc(-c4ccc(-c5cccc6oc7ccccc7c56)c5oc6ccccc6c45)c4ccccc34)n2)cc1 ; c1ccc(-c2ccc(-c3ccc4oc5cccc(-c6ccc7ccc(-c8nc(-c9ccccc9)nc(-c9ccc%10ccccc%10c9)n8)cc7c6)c5c4c3)cc2)cc1 ; c1ccc(-c2nc(-c3ccc4ccccc4c3)nc(-c3cccc4c(-c5cccc6oc7cc(-c8ccc(-c9ccccc9)c9ccccc89)ccc7c56)cccc34)n2)cc1]

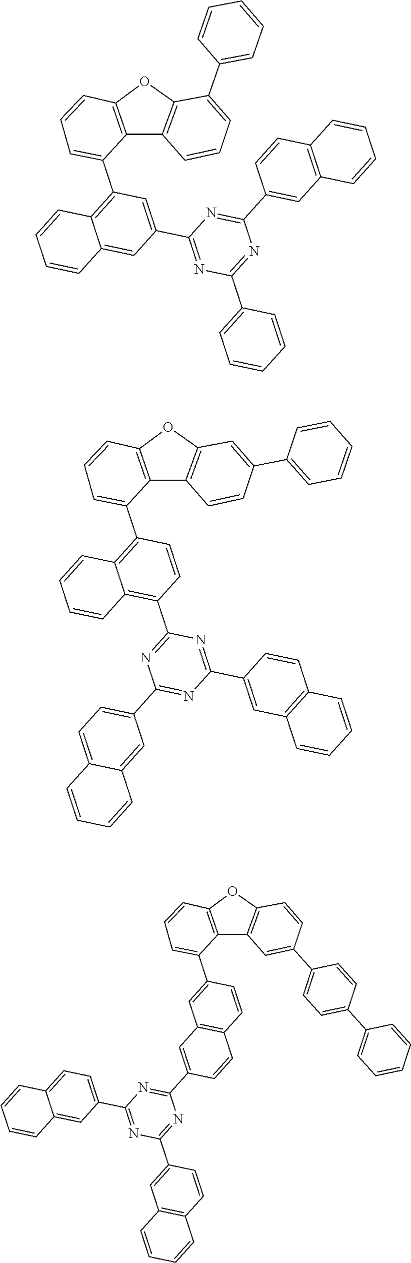 [SMILES: c1ccc(-c2nc(-c3ccc4ccccc4c3)nc(-c3cc(-c4cccc5oc6c(-c7ccccc7)cccc6c45)c4ccccc4c3)n2)cc1 ; c1ccc(-c2ccc3c(c2)oc2cccc(-c4ccc(-c5nc(-c6ccc7ccccc7c6)nc(-c6ccc7ccccc7c6)n5)c5ccccc45)c23)cc1 ; c1ccc(-c2ccc(-c3ccc4oc5cccc(-c6ccc7ccc(-c8nc(-c9ccc%10ccccc%10c9)nc(-c9ccc%10ccccc%10c9)n8)cc7c6)c5c4c3)cc2)cc1]

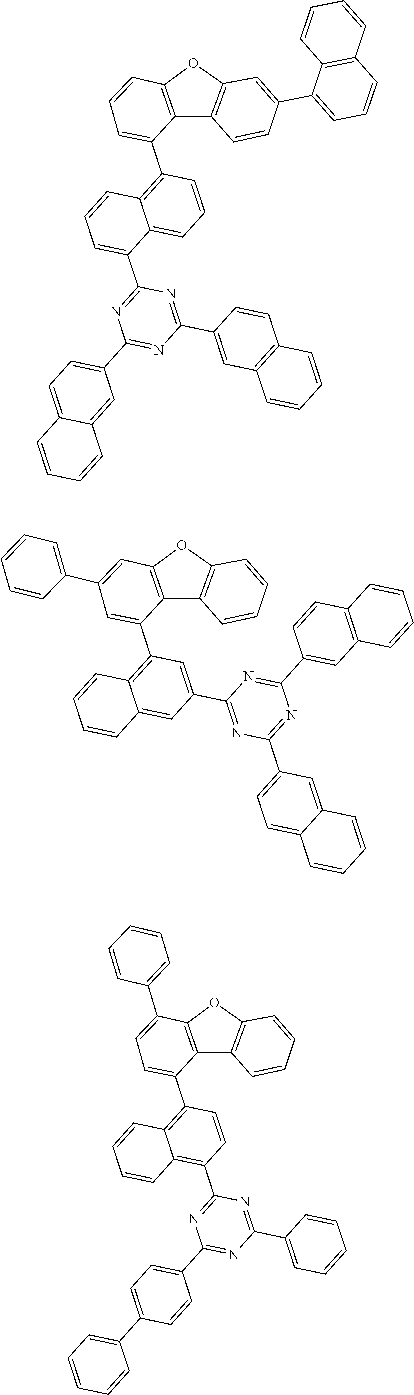 [SMILES: c1ccc2cc(-c3nc(-c4ccc5ccccc5c4)nc(-c4cccc5c(-c6cccc7oc8cc(-c9cccc%10ccccc9%10)ccc8c67)cccc45)n3)ccc2c1 ; c1ccc(-c2cc(-c3cc(-c4nc(-c5ccc6ccccc6c5)nc(-c5ccc6ccccc6c5)n4)cc4ccccc34)c3c(c2)oc2ccccc23)cc1 ; c1ccc(-c2ccc(-c3nc(-c4ccccc4)nc(-c4ccc(-c5ccc(-c6ccccc6)c6oc7ccccc7c56)c5ccccc45)n3)cc2)cc1]

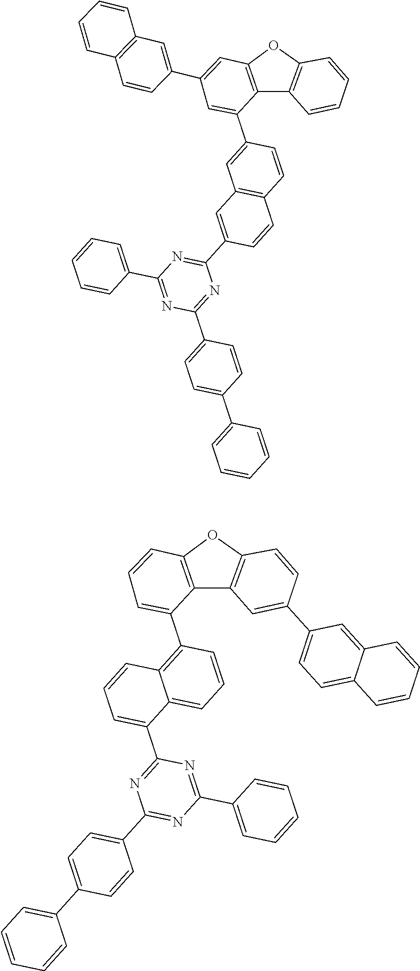 [SMILES: c1ccc(-c2ccc(-c3nc(-c4ccccc4)nc(-c4ccc5ccc(-c6cc(-c7ccc8ccccc8c7)cc7oc8ccccc8c67)cc5c4)n3)cc2)cc1 ; c1ccc(-c2ccc(-c3nc(-c4ccccc4)nc(-c4cccc5c(-c6cccc7oc8ccc(-c9ccc%10ccccc%10c9)cc8c67)cccc45)n3)cc2)cc1]

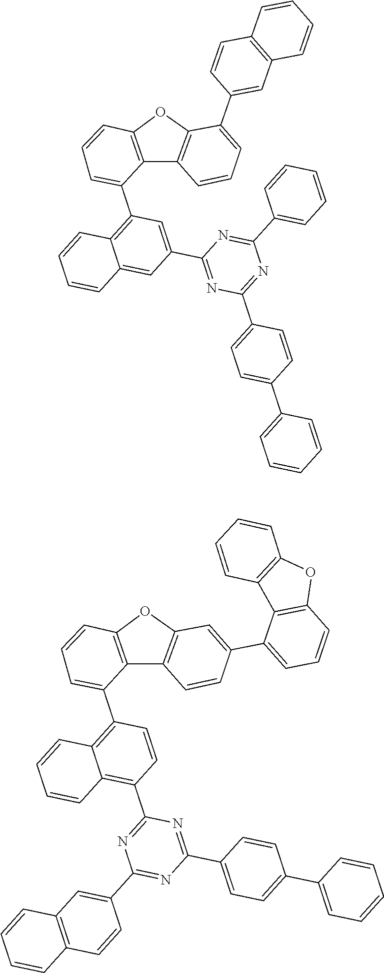 [SMILES: c1ccc(-c2ccc(-c3nc(-c4ccccc4)nc(-c4cc(-c5cccc6oc7c(-c8ccc9ccccc9c8)cccc7c56)c5ccccc5c4)n3)cc2)cc1 ; c1ccc(-c2ccc(-c3nc(-c4ccc5ccccc5c4)nc(-c4ccc(-c5cccc6oc7cc(-c8cccc9oc%10ccccc%10c89)ccc7c56)c5ccccc45)n3)cc2)cc1]

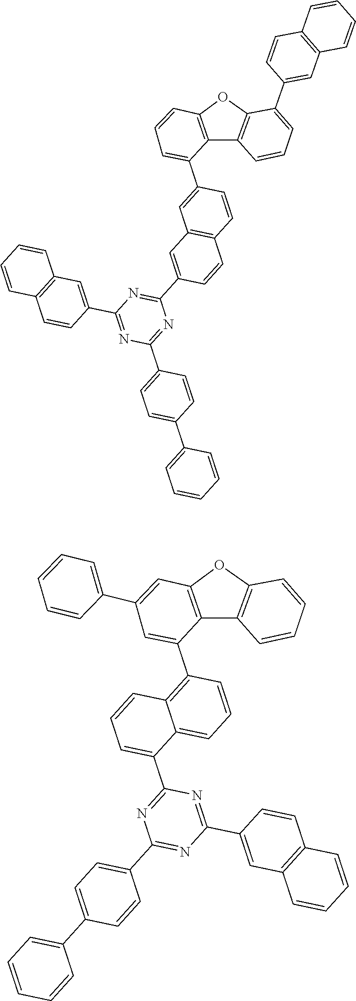 [SMILES: c1ccc(-c2ccc(-c3nc(-c4ccc5ccccc5c4)nc(-c4ccc5ccc(-c6cccc7oc8c(-c9ccc%10ccccc%10c9)cccc8c67)cc5c4)n3)cc2)cc1 ; c1ccc(-c2ccc(-c3nc(-c4ccc5ccccc5c4)nc(-c4cccc5c(-c6cc(-c7ccccc7)cc7oc8ccccc8c67)cccc45)n3)cc2)cc1]

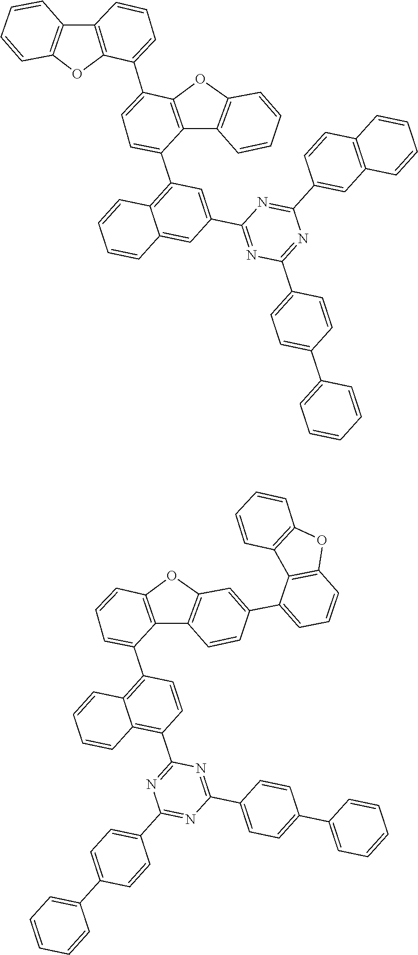 [SMILES: c1ccc(-c2ccc(-c3nc(-c4ccc5ccccc5c4)nc(-c4cc(-c5ccc(-c6cccc7c6oc6ccccc67)c6oc7ccccc7c56)c5ccccc5c4)n3)cc2)cc1 ; c1ccc(-c2ccc(-c3nc(-c4ccc(-c5ccccc5)cc4)nc(-c4ccc(-c5cccc6oc7cc(-c8cccc9oc%10ccccc%10c89)ccc7c56)c5ccccc45)n3)cc2)cc1]

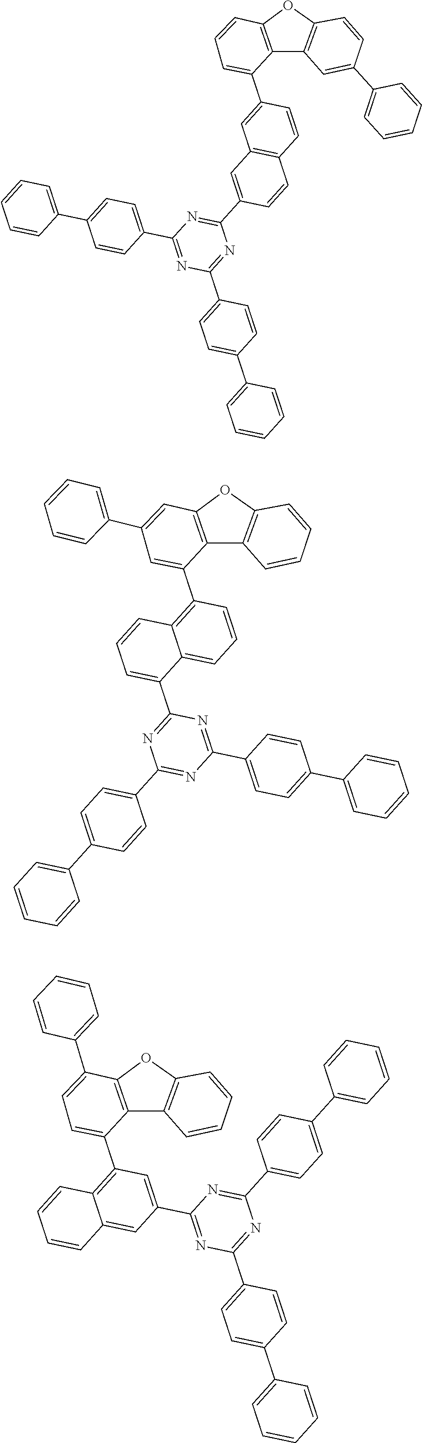 [SMILES: c1ccc(-c2ccc(-c3nc(-c4ccc(-c5ccccc5)cc4)nc(-c4ccc5ccc(-c6cccc7oc8ccc(-c9ccccc9)cc8c67)cc5c4)n3)cc2)cc1 ; c1ccc(-c2ccc(-c3nc(-c4ccc(-c5ccccc5)cc4)nc(-c4cccc5c(-c6cc(-c7ccccc7)cc7oc8ccccc8c67)cccc45)n3)cc2)cc1 ; c1ccc(-c2ccc(-c3nc(-c4ccc(-c5ccccc5)cc4)nc(-c4cc(-c5ccc(-c6ccccc6)c6oc7ccccc7c56)c5ccccc5c4)n3)cc2)cc1]

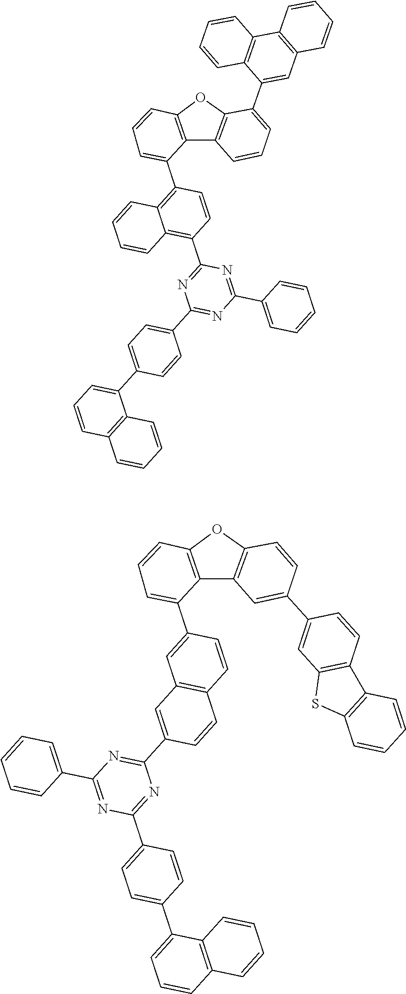 [SMILES: c1ccc(-c2nc(-c3ccc(-c4cccc5ccccc45)cc3)nc(-c3ccc(-c4cccc5oc6c(-c7cc8ccccc8c8ccccc78)cccc6c45)c4ccccc34)n2)cc1 ; c1ccc(-c2nc(-c3ccc(-c4cccc5ccccc45)cc3)nc(-c3ccc4ccc(-c5cccc6oc7ccc(-c8ccc9c(c8)sc8ccccc89)cc7c56)cc4c3)n2)cc1]

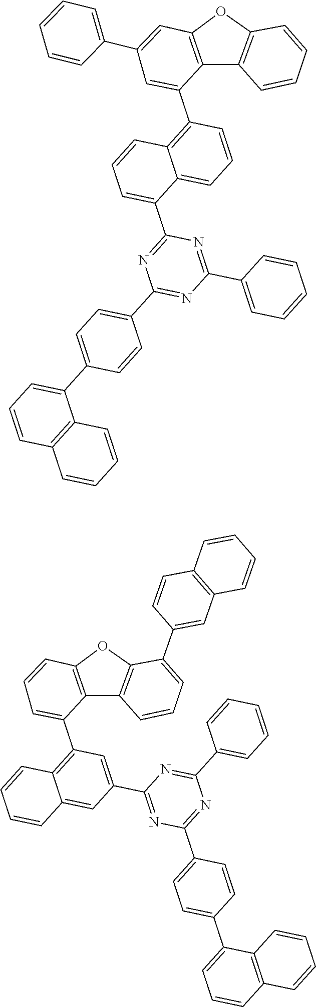 [SMILES: c1ccc(-c2cc(-c3cccc4c(-c5nc(-c6ccccc6)nc(-c6ccc(-c7cccc8ccccc78)cc6)n5)cccc34)c3c(c2)oc2ccccc23)cc1 ; c1ccc(-c2nc(-c3ccc(-c4cccc5ccccc45)cc3)nc(-c3cc(-c4cccc5oc6c(-c7ccc8ccccc8c7)cccc6c45)c4ccccc4c3)n2)cc1]

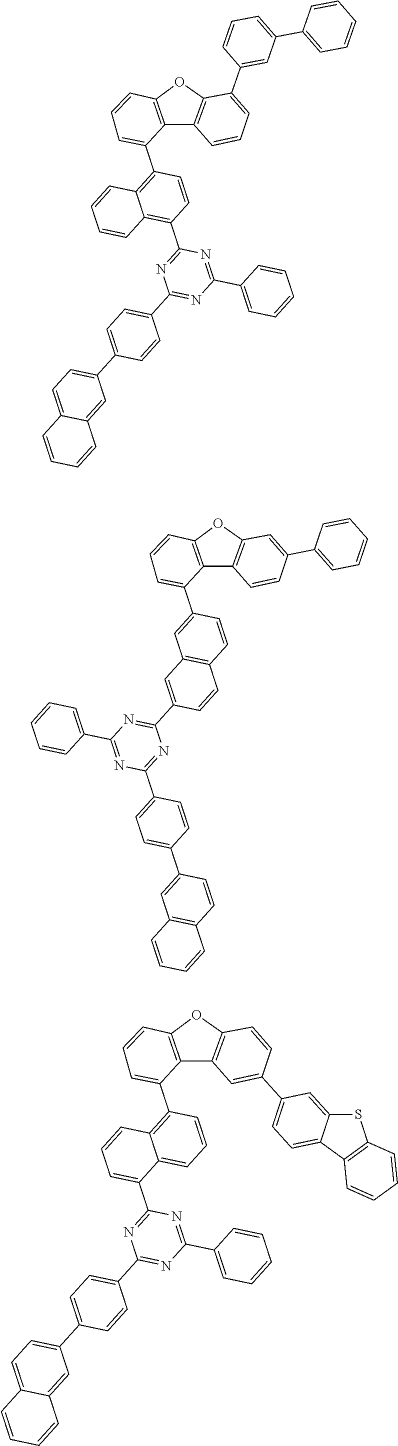 [SMILES: c1ccc(-c2cccc(-c3cccc4c3oc3cccc(-c5ccc(-c6nc(-c7ccccc7)nc(-c7ccc(-c8ccc9ccccc9c8)cc7)n6)c6ccccc56)c34)c2)cc1 ; c1ccc(-c2ccc3c(c2)oc2cccc(-c4ccc5ccc(-c6nc(-c7ccccc7)nc(-c7ccc(-c8ccc9ccccc9c8)cc7)n6)cc5c4)c23)cc1 ; c1ccc(-c2nc(-c3ccc(-c4ccc5ccccc5c4)cc3)nc(-c3cccc4c(-c5cccc6oc7ccc(-c8ccc9c(c8)sc8ccccc89)cc7c56)cccc34)n2)cc1]

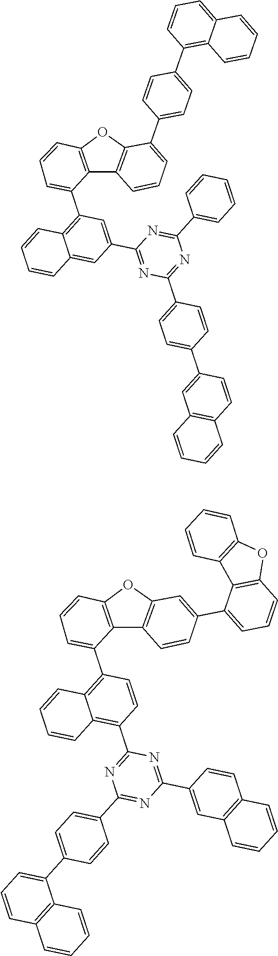 [SMILES: c1ccc(-c2nc(-c3ccc(-c4ccc5ccccc5c4)cc3)nc(-c3cc(-c4cccc5oc6c(-c7ccc(-c8cccc9ccccc89)cc7)cccc6c45)c4ccccc4c3)n2)cc1 ; c1ccc2cc(-c3nc(-c4ccc(-c5cccc6ccccc56)cc4)nc(-c4ccc(-c5cccc6oc7cc(-c8cccc9oc%10ccccc%10c89)ccc7c56)c5ccccc45)n3)ccc2c1]

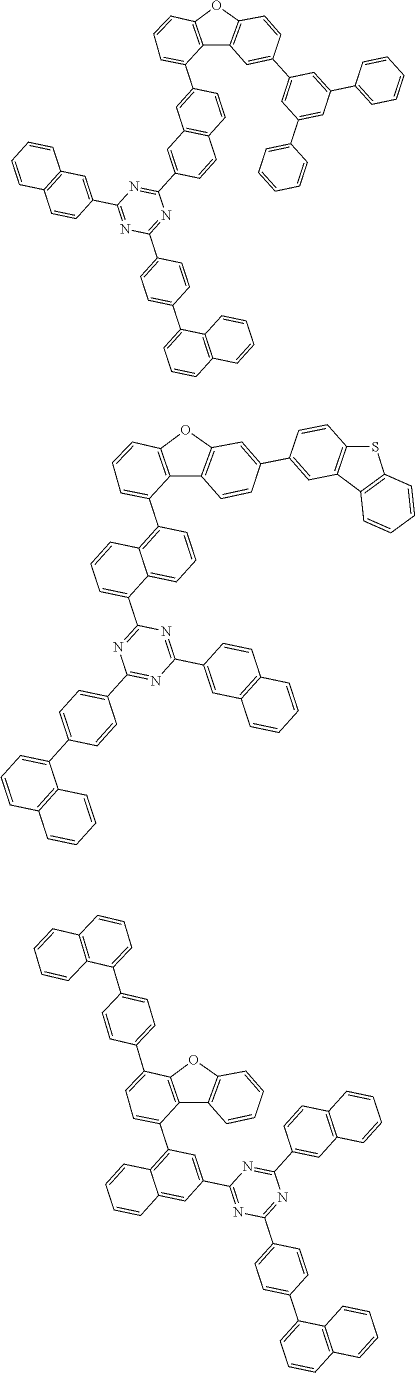 [SMILES: c1ccc(-c2cc(-c3ccccc3)cc(-c3ccc4oc5cccc(-c6ccc7ccc(-c8nc(-c9ccc(-c%10cccc%11ccccc%10%11)cc9)nc(-c9ccc%10ccccc%10c9)n8)cc7c6)c5c4c3)c2)cc1 ; c1ccc2cc(-c3nc(-c4ccc(-c5cccc6ccccc56)cc4)nc(-c4cccc5c(-c6cccc7oc8cc(-c9ccc%10sc%11ccccc%11c%10c9)ccc8c67)cccc45)n3)ccc2c1 ; c1ccc2cc(-c3nc(-c4ccc(-c5cccc6ccccc56)cc4)nc(-c4cc(-c5ccc(-c6ccc(-c7cccc8ccccc78)cc6)c6oc7ccccc7c56)c5ccccc5c4)n3)ccc2c1]

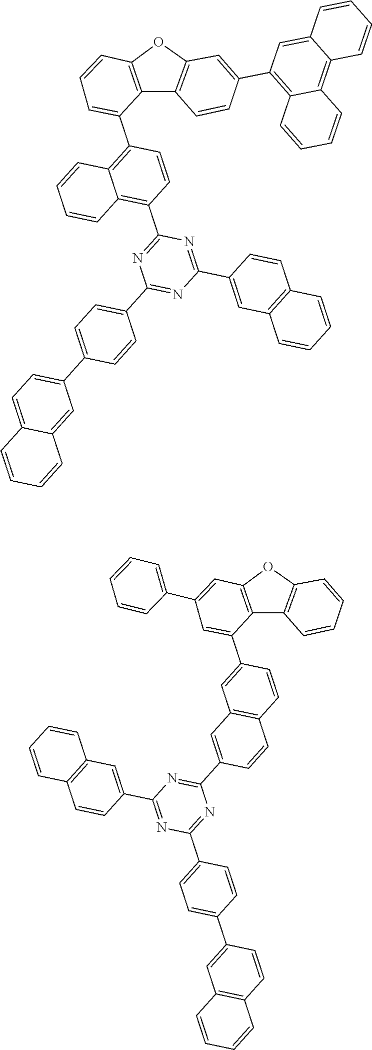 [SMILES: c1ccc2cc(-c3ccc(-c4nc(-c5ccc6ccccc6c5)nc(-c5ccc(-c6cccc7oc8cc(-c9cc%10ccccc%10c%10ccccc9%10)ccc8c67)c6ccccc56)n4)cc3)ccc2c1 ; c1ccc(-c2cc(-c3ccc4ccc(-c5nc(-c6ccc(-c7ccc8ccccc8c7)cc6)nc(-c6ccc7ccccc7c6)n5)cc4c3)c3c(c2)oc2ccccc23)cc1]

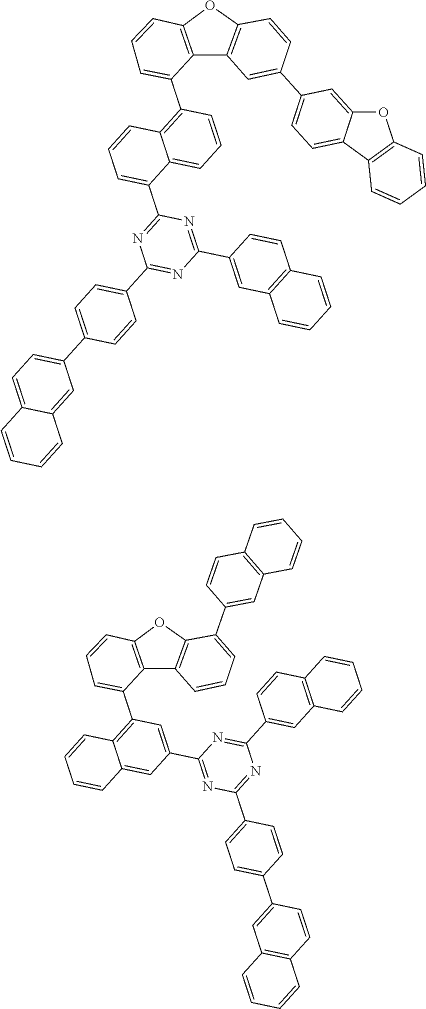 [SMILES: c1ccc2cc(-c3ccc(-c4nc(-c5ccc6ccccc6c5)nc(-c5cccc6c(-c7cccc8oc9ccc(-c%10ccc%11c(c%10)oc%10ccccc%10%11)cc9c78)cccc56)n4)cc3)ccc2c1 ; c1ccc2cc(-c3ccc(-c4nc(-c5ccc6ccccc6c5)nc(-c5cc(-c6cccc7oc8c(-c9ccc%10ccccc%10c9)cccc8c67)c6ccccc6c5)n4)cc3)ccc2c1]

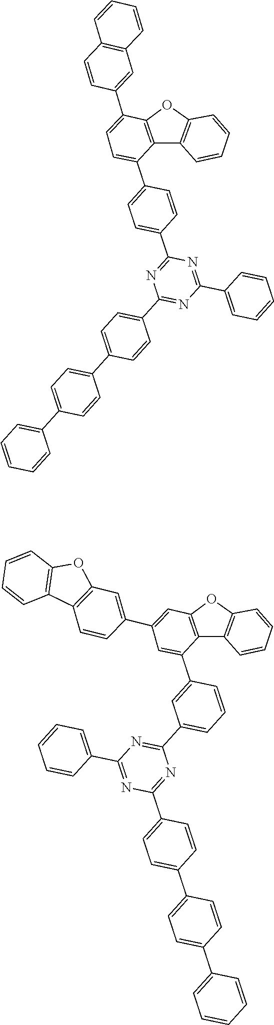 [SMILES: c1ccc(-c2ccc(-c3ccc(-c4nc(-c5ccccc5)nc(-c5ccc(-c6ccc(-c7ccc8ccccc8c7)c7oc8ccccc8c67)cc5)n4)cc3)cc2)cc1 ; c1ccc(-c2ccc(-c3ccc(-c4nc(-c5ccccc5)nc(-c5cccc(-c6cc(-c7ccc8c(c7)oc7ccccc78)cc7oc8ccccc8c67)c5)n4)cc3)cc2)cc1]

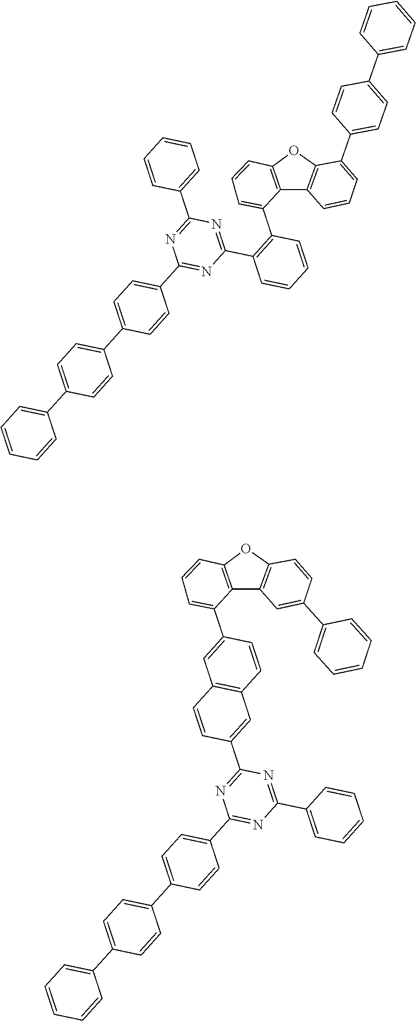 [SMILES: c1ccc(-c2ccc(-c3ccc(-c4nc(-c5ccccc5)nc(-c5ccccc5-c5cccc6oc7c(-c8ccc(-c9ccccc9)cc8)cccc7c56)n4)cc3)cc2)cc1 ; c1ccc(-c2ccc(-c3ccc(-c4nc(-c5ccccc5)nc(-c5ccc6cc(-c7cccc8oc9ccc(-c%10ccccc%10)cc9c78)ccc6c5)n4)cc3)cc2)cc1]

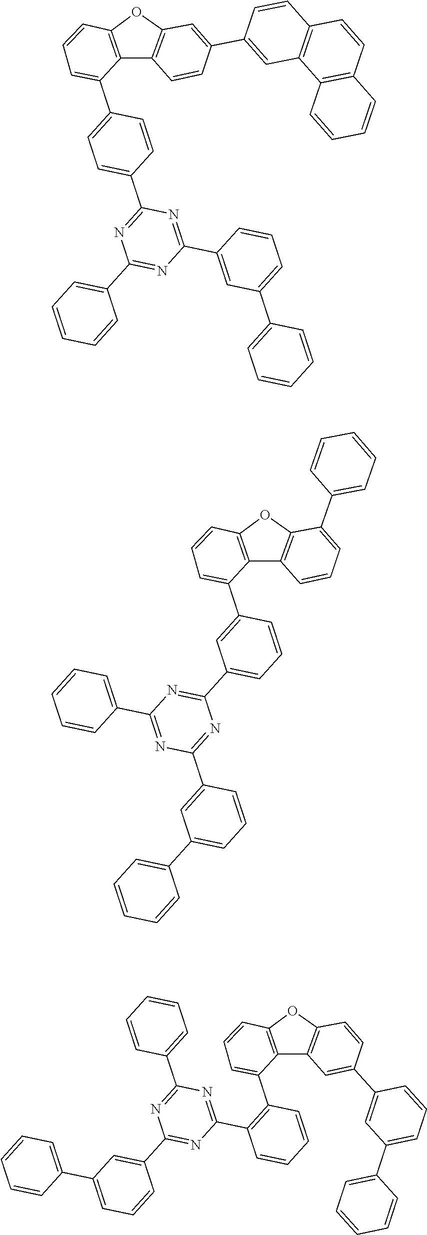 [SMILES: c1ccc(-c2cccc(-c3nc(-c4ccccc4)nc(-c4ccc(-c5cccc6oc7cc(-c8ccc9ccc%10ccccc%10c9c8)ccc7c56)cc4)n3)c2)cc1 ; c1ccc(-c2cccc(-c3nc(-c4ccccc4)nc(-c4cccc(-c5cccc6oc7c(-c8ccccc8)cccc7c56)c4)n3)c2)cc1 ; c1ccc(-c2cccc(-c3ccc4oc5cccc(-c6ccccc6-c6nc(-c7ccccc7)nc(-c7cccc(-c8ccccc8)c7)n6)c5c4c3)c2)cc1]

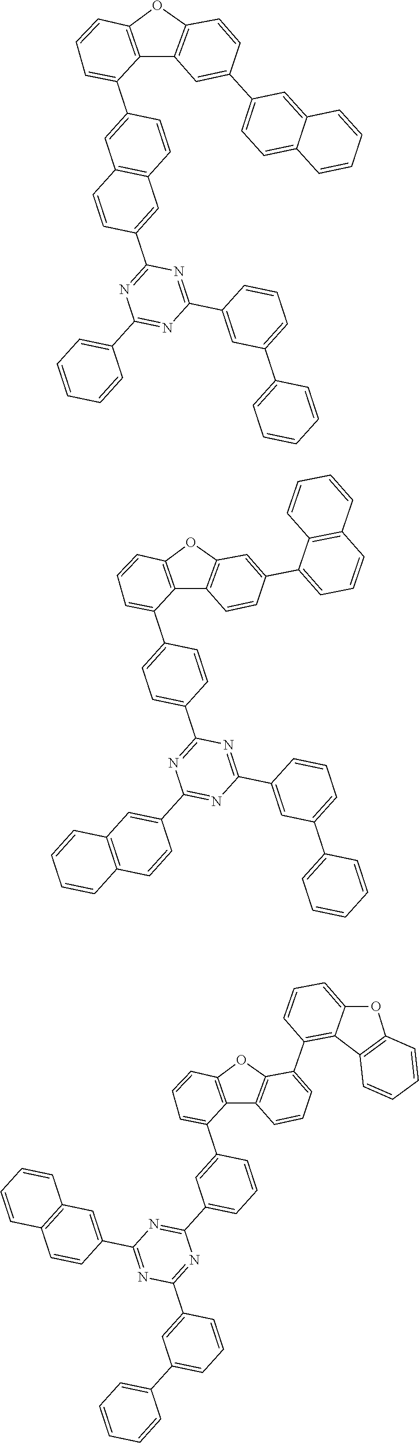

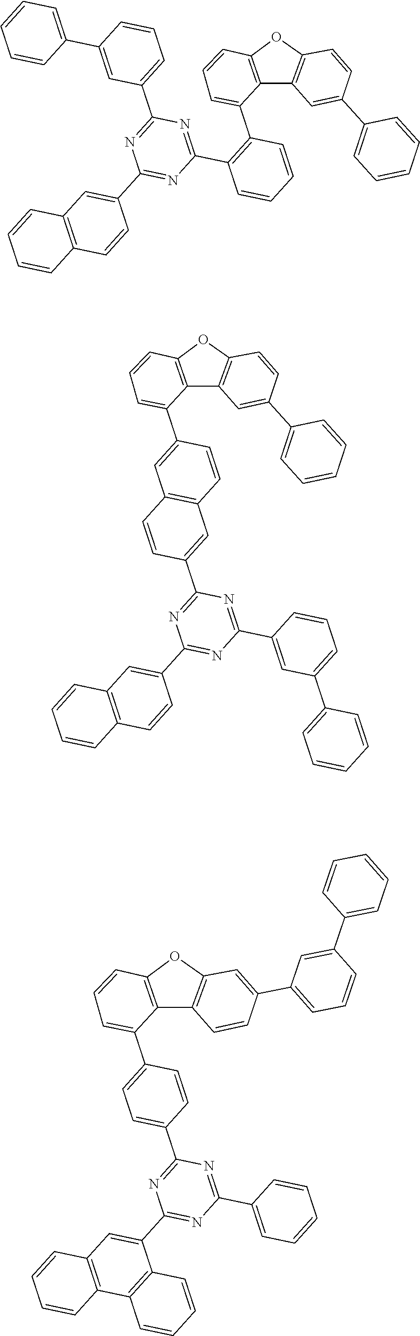 [SMILES: c1ccc(-c2cccc(-c3nc(-c4ccc5ccccc5c4)nc(-c4ccccc4-c4cccc5oc6ccc(-c7ccccc7)cc6c45)n3)c2)cc1 ; c1ccc(-c2cccc(-c3nc(-c4ccc5ccccc5c4)nc(-c4ccc5cc(-c6cccc7oc8ccc(-c9ccccc9)cc8c67)ccc5c4)n3)c2)cc1 ; c1ccc(-c2cccc(-c3ccc4c(c3)oc3cccc(-c5ccc(-c6nc(-c7ccccc7)nc(-c7cc8ccccc8c8ccccc78)n6)cc5)c34)c2)cc1]

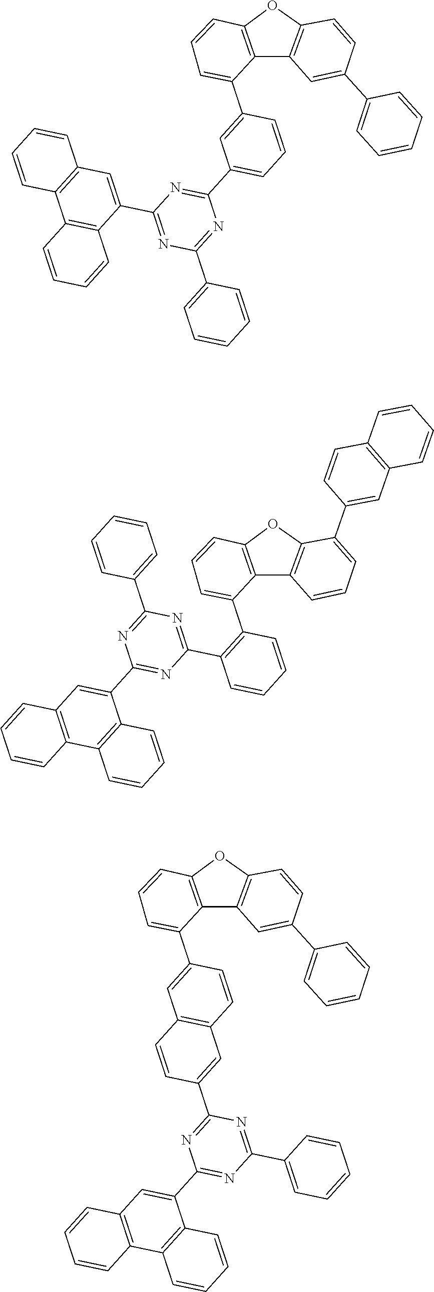 [SMILES: c1ccc(-c2ccc3oc4cccc(-c5cccc(-c6nc(-c7ccccc7)nc(-c7cc8ccccc8c8ccccc78)n6)c5)c4c3c2)cc1 ; c1ccc(-c2nc(-c3ccccc3-c3cccc4oc5c(-c6ccc7ccccc7c6)cccc5c34)nc(-c3cc4ccccc4c4ccccc34)n2)cc1 ; c1ccc(-c2ccc3oc4cccc(-c5ccc6cc(-c7nc(-c8ccccc8)nc(-c8cc9ccccc9c9ccccc89)n7)ccc6c5)c4c3c2)cc1]

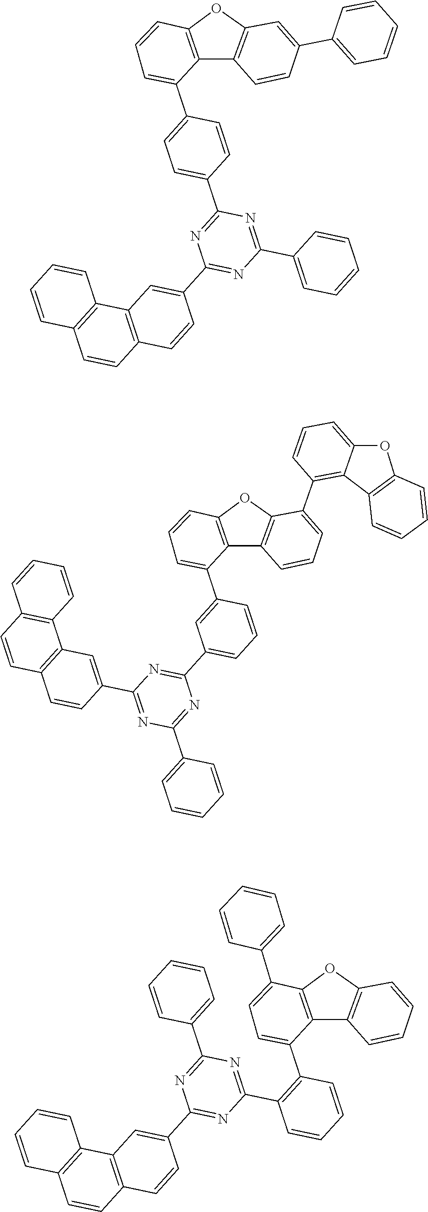 [SMILES: c1ccc(-c2ccc3c(c2)oc2cccc(-c4ccc(-c5nc(-c6ccccc6)nc(-c6ccc7ccc8ccccc8c7c6)n5)cc4)c23)cc1 ; c1ccc(-c2nc(-c3cccc(-c4cccc5oc6c(-c7cccc8oc9ccccc9c78)cccc6c45)c3)nc(-c3ccc4ccc5ccccc5c4c3)n2)cc1 ; c1ccc(-c2nc(-c3ccc4ccc5ccccc5c4c3)nc(-c3ccccc3-c3ccc(-c4ccccc4)c4oc5ccccc5c34)n2)cc1]

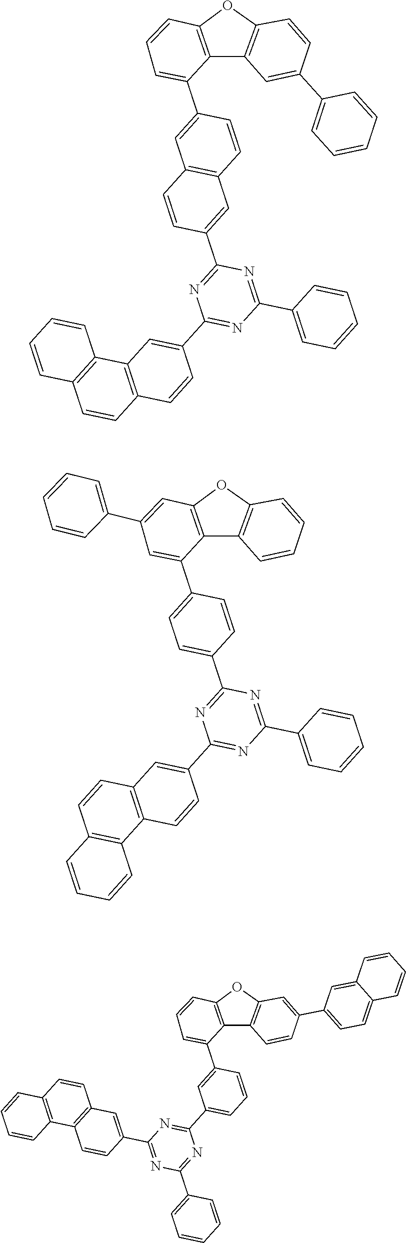 [SMILES: c1ccc(-c2ccc3oc4cccc(-c5ccc6cc(-c7nc(-c8ccccc8)nc(-c8ccc9ccc%10ccccc%10c9c8)n7)ccc6c5)c4c3c2)cc1 ; c1ccc(-c2cc(-c3ccc(-c4nc(-c5ccccc5)nc(-c5ccc6c(ccc7ccccc76)c5)n4)cc3)c3c(c2)oc2ccccc23)cc1 ; c1ccc(-c2nc(-c3cccc(-c4cccc5oc6cc(-c7ccc8ccccc8c7)ccc6c45)c3)nc(-c3ccc4c(ccc5ccccc54)c3)n2)cc1]

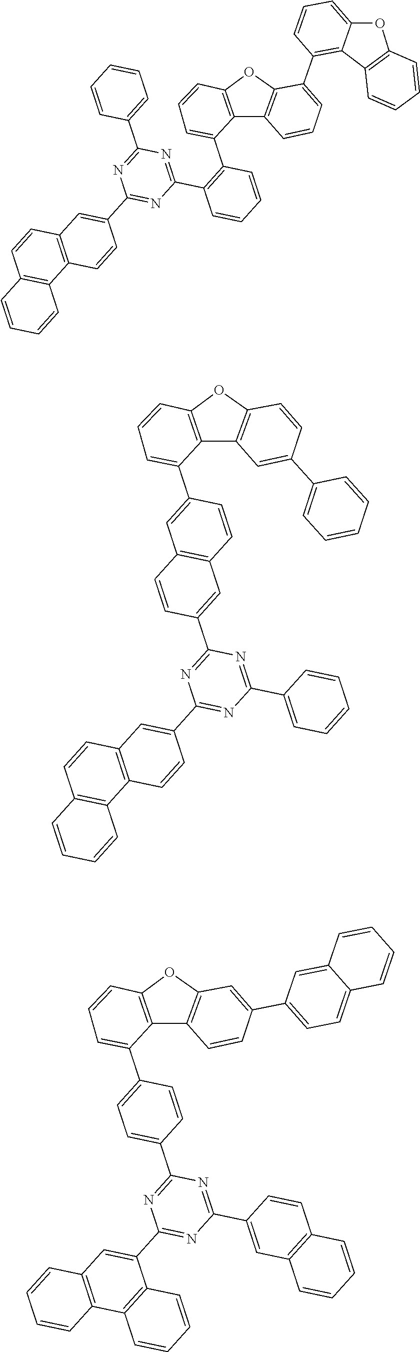 [SMILES: c1ccc(-c2nc(-c3ccc4c(ccc5ccccc54)c3)nc(-c3ccccc3-c3cccc4oc5c(-c6cccc7oc8ccccc8c67)cccc5c34)n2)cc1 ; c1ccc(-c2ccc3oc4cccc(-c5ccc6cc(-c7nc(-c8ccccc8)nc(-c8ccc9c(ccc%10ccccc%109)c8)n7)ccc6c5)c4c3c2)cc1 ; c1ccc2cc(-c3ccc4c(c3)oc3cccc(-c5ccc(-c6nc(-c7ccc8ccccc8c7)nc(-c7cc8ccccc8c8ccccc78)n6)cc5)c34)ccc2c1]

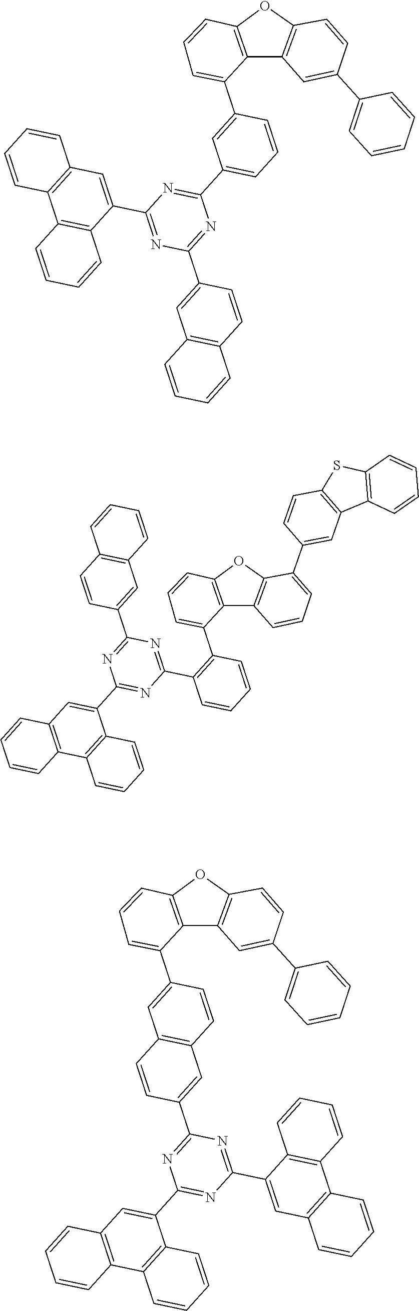 [SMILES: c1ccc(-c2ccc3oc4cccc(-c5cccc(-c6nc(-c7ccc8ccccc8c7)nc(-c7cc8ccccc8c8ccccc78)n6)c5)c4c3c2)cc1 ; c1ccc(-c2cccc3oc4c(-c5ccc6sc7ccccc7c6c5)cccc4c23)c(-c2nc(-c3ccc4ccccc4c3)nc(-c3cc4ccccc4c4ccccc34)n2)c1 ; c1ccc(-c2ccc3oc4cccc(-c5ccc6cc(-c7nc(-c8cc9ccccc9c9ccccc89)nc(-c8cc9ccccc9c9ccccc89)n7)ccc6c5)c4c3c2)cc1]

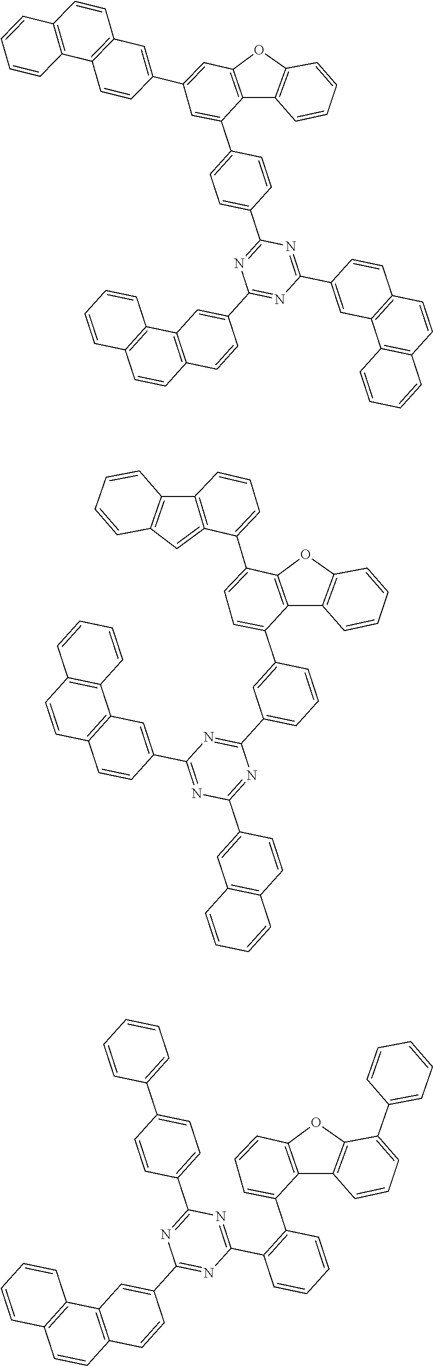

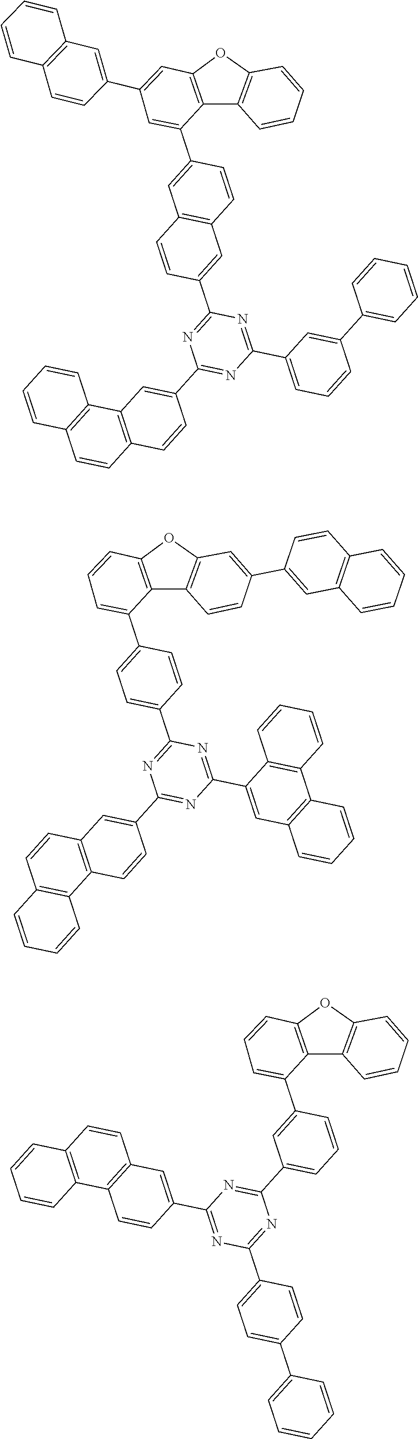 [SMILES: c1ccc(-c2cccc(-c3nc(-c4ccc5cc(-c6cc(-c7ccc8ccccc8c7)cc7oc8ccccc8c67)ccc5c4)nc(-c4ccc5ccc6ccccc6c5c4)n3)c2)cc1 ; c1ccc2cc(-c3ccc4c(c3)oc3cccc(-c5ccc(-c6nc(-c7ccc8c(ccc9ccccc98)c7)nc(-c7cc8ccccc8c8ccccc78)n6)cc5)c34)ccc2c1 ; c1ccc(-c2ccc(-c3nc(-c4cccc(-c5cccc6oc7ccccc7c56)c4)nc(-c4ccc5c(ccc6ccccc65)c4)n3)cc2)cc1]

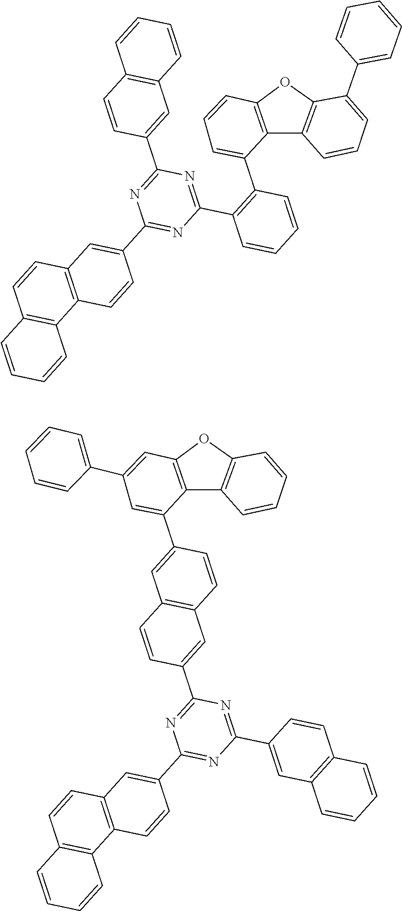

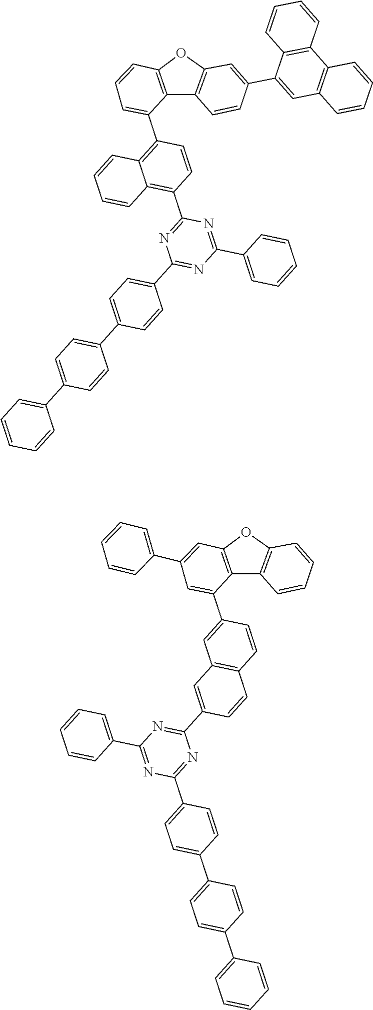 [SMILES: c1ccc(-c2ccc(-c3ccc(-c4nc(-c5ccccc5)nc(-c5ccc(-c6cccc7oc8cc(-c9cc%10ccccc%10c%10ccccc9%10)ccc8c67)c6ccccc56)n4)cc3)cc2)cc1 ; c1ccc(-c2ccc(-c3ccc(-c4nc(-c5ccccc5)nc(-c5ccc6ccc(-c7cc(-c8ccccc8)cc8oc9ccccc9c78)cc6c5)n4)cc3)cc2)cc1]

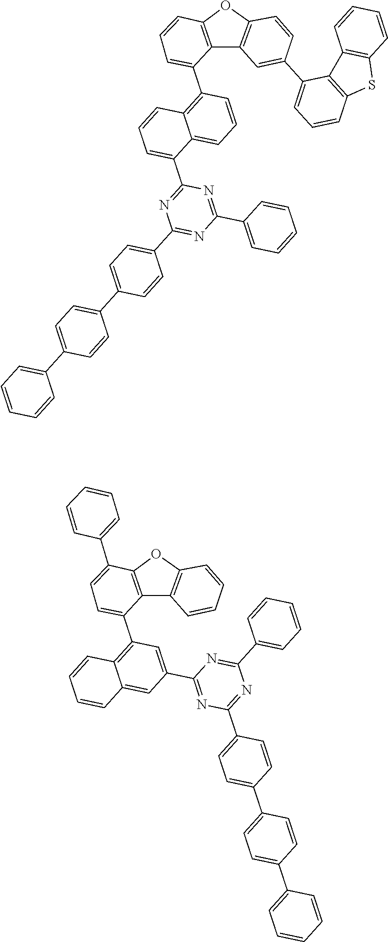 [SMILES: c1ccc(-c2ccc(-c3ccc(-c4nc(-c5ccccc5)nc(-c5cccc6c(-c7cccc8oc9ccc(-c%10cccc%11sc%12ccccc%12c%10%11)cc9c78)cccc56)n4)cc3)cc2)cc1 ; c1ccc(-c2ccc(-c3ccc(-c4nc(-c5ccccc5)nc(-c5cc(-c6ccc(-c7ccccc7)c7oc8ccccc8c67)c6ccccc6c5)n4)cc3)cc2)cc1]

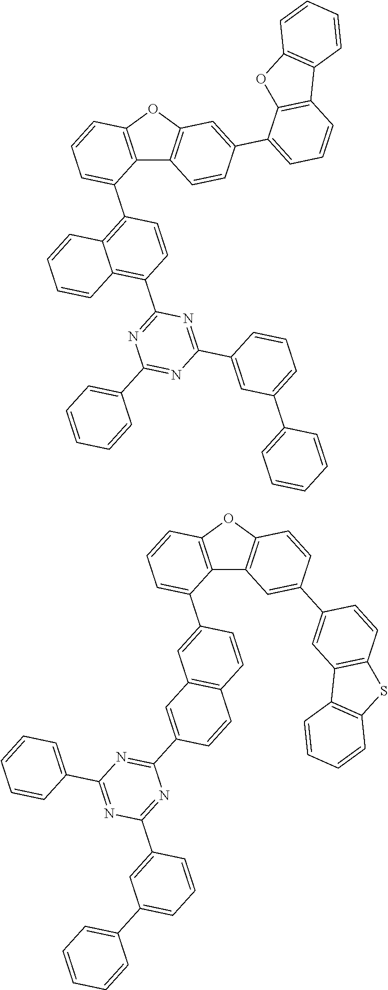

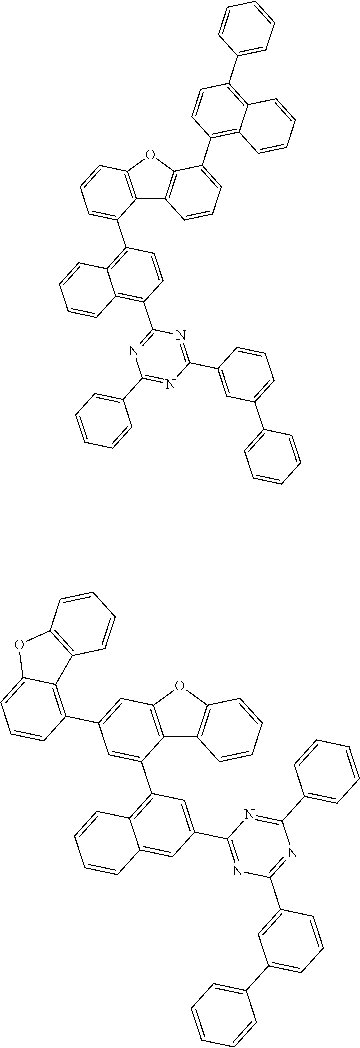 [SMILES: c1ccc(-c2cccc(-c3nc(-c4ccccc4)nc(-c4ccc(-c5cccc6oc7c(-c8ccc(-c9ccccc9)c9ccccc89)cccc7c56)c5ccccc45)n3)c2)cc1 ; c1ccc(-c2cccc(-c3nc(-c4ccccc4)nc(-c4cc(-c5cc(-c6cccc7oc8ccccc8c67)cc6oc7ccccc7c56)c5ccccc5c4)n3)c2)cc1]

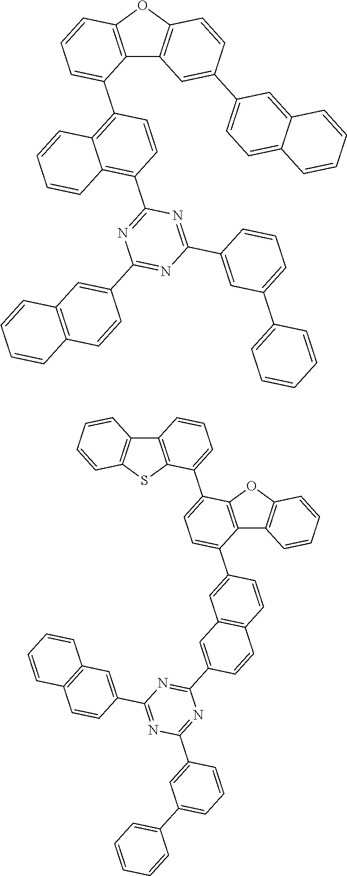 [SMILES: c1ccc(-c2cccc(-c3nc(-c4ccc5ccccc5c4)nc(-c4ccc(-c5cccc6oc7ccc(-c8ccc9ccccc9c8)cc7c56)c5ccccc45)n3)c2)cc1 ; c1ccc(-c2cccc(-c3nc(-c4ccc5ccccc5c4)nc(-c4ccc5ccc(-c6ccc(-c7cccc8c7sc7ccccc78)c7oc8ccccc8c67)cc5c4)n3)c2)cc1]

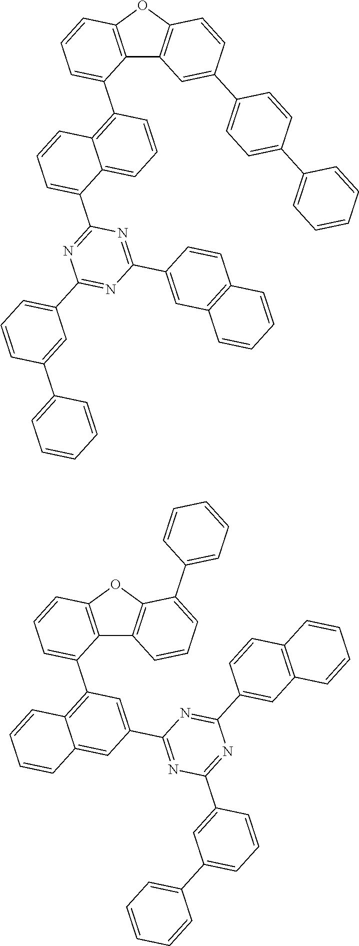

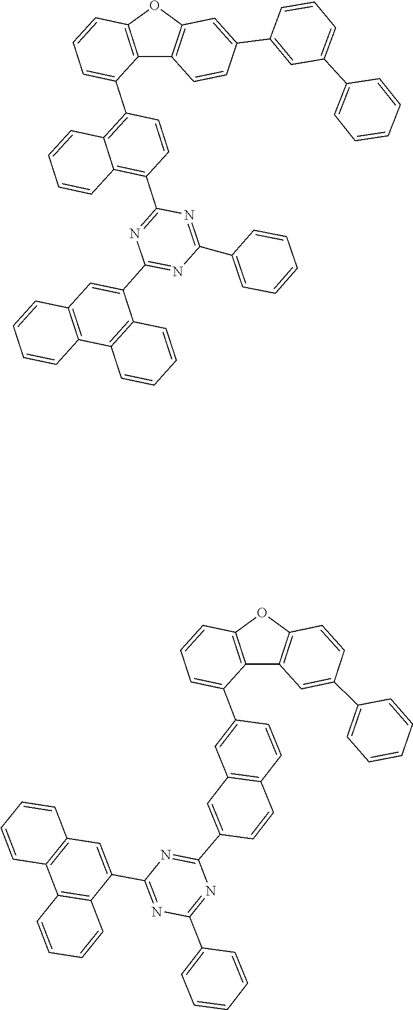 [SMILES: c1ccc(-c2cccc(-c3ccc4c(c3)oc3cccc(-c5ccc(-c6nc(-c7ccccc7)nc(-c7cc8ccccc8c8ccccc78)n6)c6ccccc56)c34)c2)cc1 ; c1ccc(-c2ccc3oc4cccc(-c5ccc6ccc(-c7nc(-c8ccccc8)nc(-c8cc9ccccc9c9ccccc89)n7)cc6c5)c4c3c2)cc1]

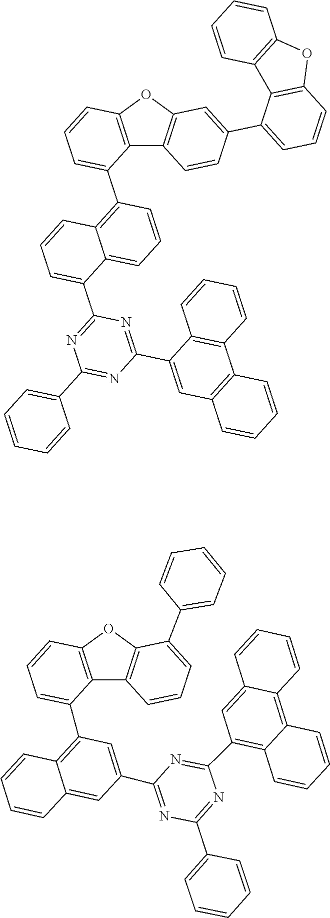 [SMILES: c1ccc(-c2nc(-c3cccc4c(-c5cccc6oc7cc(-c8cccc9oc%10ccccc%10c89)ccc7c56)cccc34)nc(-c3cc4ccccc4c4ccccc34)n2)cc1 ; c1ccc(-c2nc(-c3cc(-c4cccc5oc6c(-c7ccccc7)cccc6c45)c4ccccc4c3)nc(-c3cc4ccccc4c4ccccc34)n2)cc1]

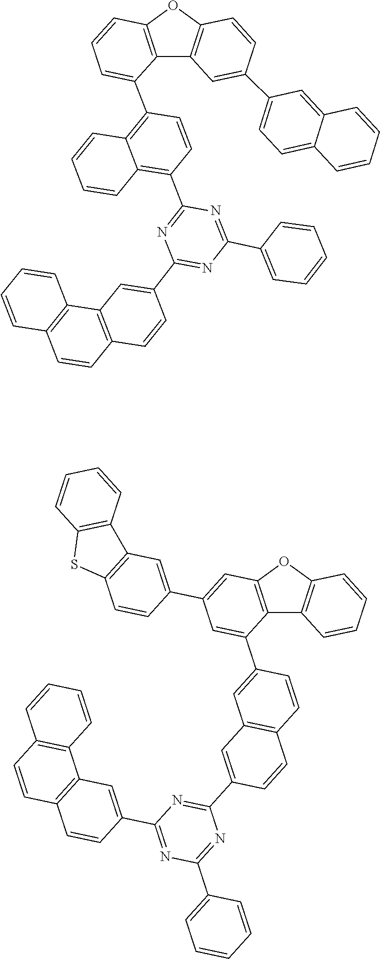 [SMILES: c1ccc(-c2nc(-c3ccc4ccc5ccccc5c4c3)nc(-c3ccc(-c4cccc5oc6ccc(-c7ccc8ccccc8c7)cc6c45)c4ccccc34)n2)cc1 ; c1ccc(-c2nc(-c3ccc4ccc(-c5cc(-c6ccc7sc8ccccc8c7c6)cc6oc7ccccc7c56)cc4c3)nc(-c3ccc4ccc5ccccc5c4c3)n2)cc1]

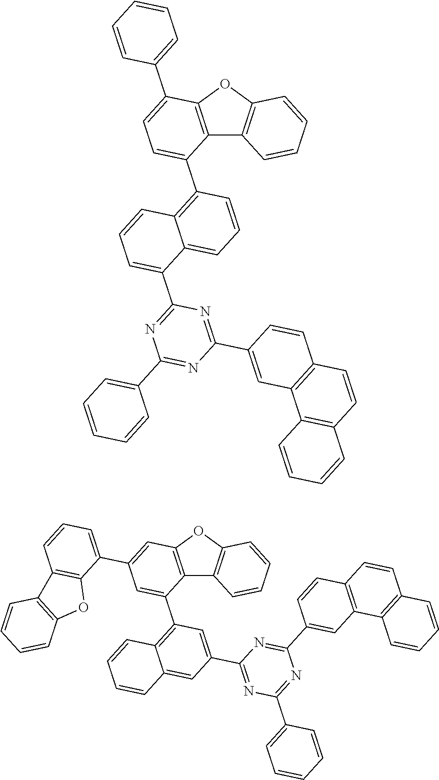

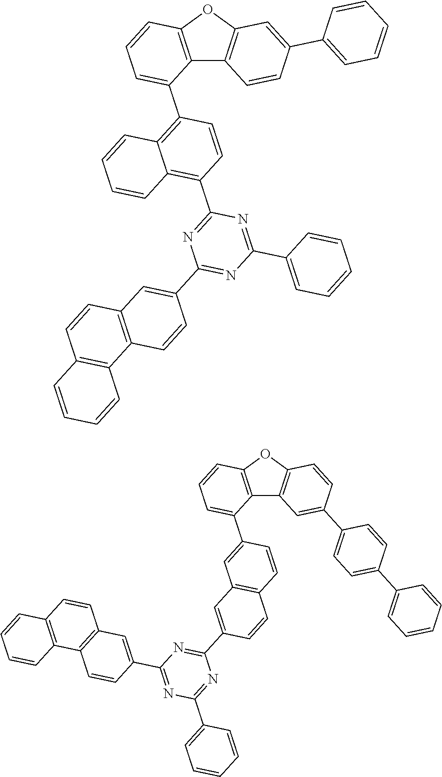 [SMILES: c1ccc(-c2ccc3c(c2)oc2cccc(-c4ccc(-c5nc(-c6ccccc6)nc(-c6ccc7c(ccc8ccccc87)c6)n5)c5ccccc45)c23)cc1 ; c1ccc(-c2ccc(-c3ccc4oc5cccc(-c6ccc7ccc(-c8nc(-c9ccccc9)nc(-c9ccc%10c(ccc%11ccccc%11%10)c9)n8)cc7c6)c5c4c3)cc2)cc1]

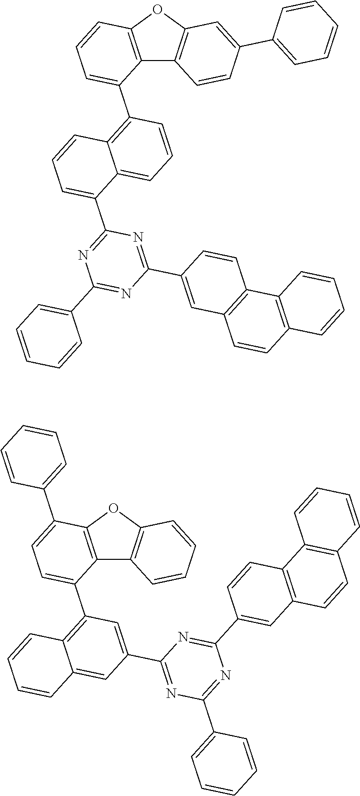 [SMILES: c1ccc(-c2ccc3c(c2)oc2cccc(-c4cccc5c(-c6nc(-c7ccccc7)nc(-c7ccc8c(ccc9ccccc98)c7)n6)cccc45)c23)cc1 ; c1ccc(-c2nc(-c3cc(-c4ccc(-c5ccccc5)c5oc6ccccc6c45)c4ccccc4c3)nc(-c3ccc4c(ccc5ccccc54)c3)n2)cc1]

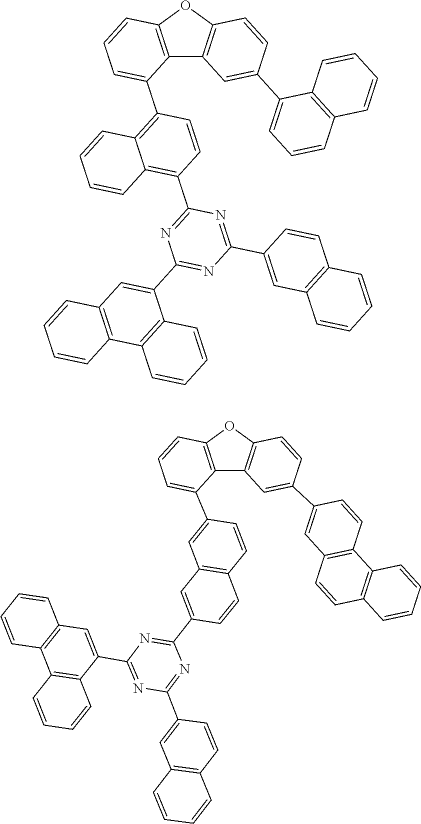 [SMILES: c1ccc2cc(-c3nc(-c4ccc(-c5cccc6oc7ccc(-c8cccc9ccccc89)cc7c56)c5ccccc45)nc(-c4cc5ccccc5c5ccccc45)n3)ccc2c1 ; c1ccc2cc(-c3nc(-c4ccc5ccc(-c6cccc7oc8ccc(-c9ccc%10c(ccc%11ccccc%11%10)c9)cc8c67)cc5c4)nc(-c4cc5ccccc5c5ccccc45)n3)ccc2c1]

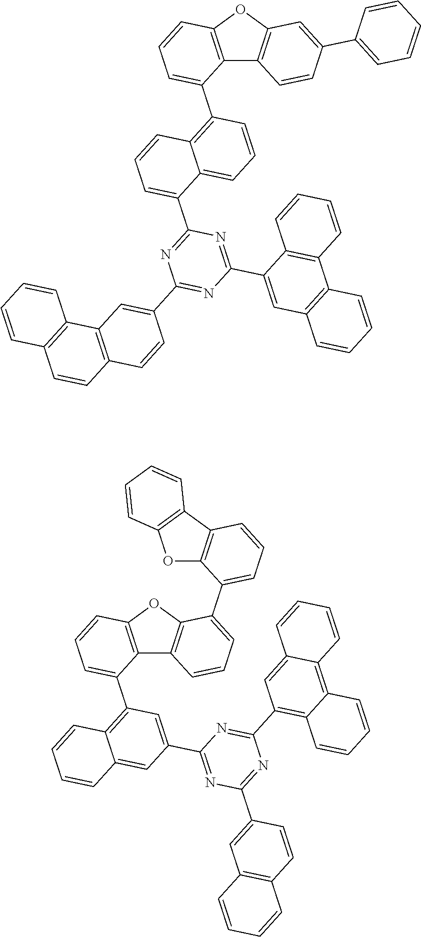 [SMILES: c1ccc(-c2ccc3c(c2)oc2cccc(-c4cccc5c(-c6nc(-c7ccc8ccc9ccccc9c8c7)nc(-c7cc8ccccc8c8ccccc78)n6)cccc45)c23)cc1 ; c1ccc2cc(-c3nc(-c4cc(-c5cccc6oc7c(-c8cccc9c8oc8ccccc89)cccc7c56)c5ccccc5c4)nc(-c4cc5ccccc5c5ccccc45)n3)ccc2c1]

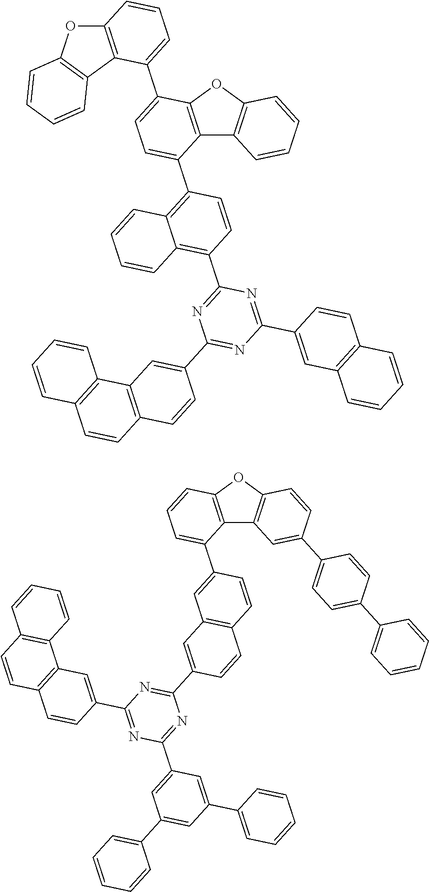 [SMILES: c1ccc2cc(-c3nc(-c4ccc5ccc6ccccc6c5c4)nc(-c4ccc(-c5ccc(-c6cccc7oc8ccccc8c67)c6oc7ccccc7c56)c5ccccc45)n3)ccc2c1 ; c1ccc(-c2ccc(-c3ccc4oc5cccc(-c6ccc7ccc(-c8nc(-c9cc(-c%10ccccc%10)cc(-c%10ccccc%10)c9)nc(-c9ccc%10ccc%11ccccc%11c%10c9)n8)cc7c6)c5c4c3)cc2)cc1]

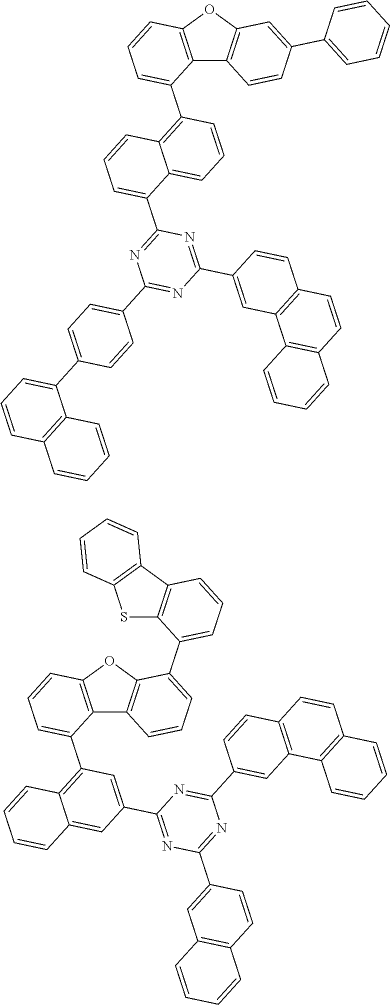

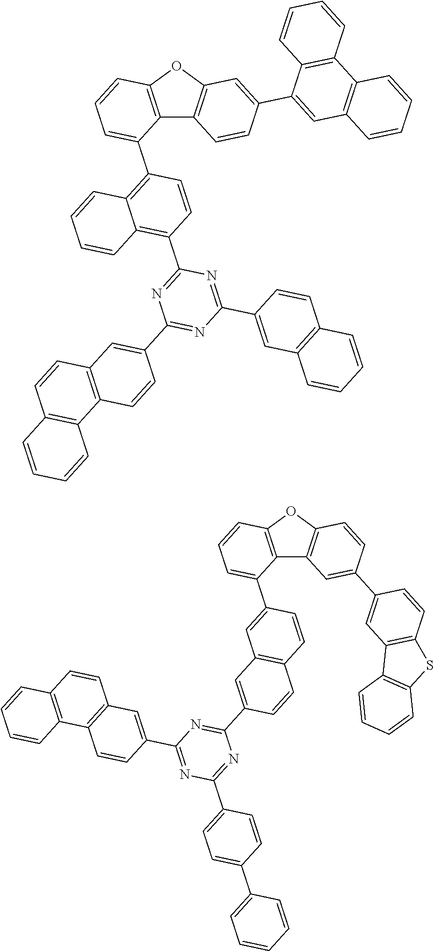

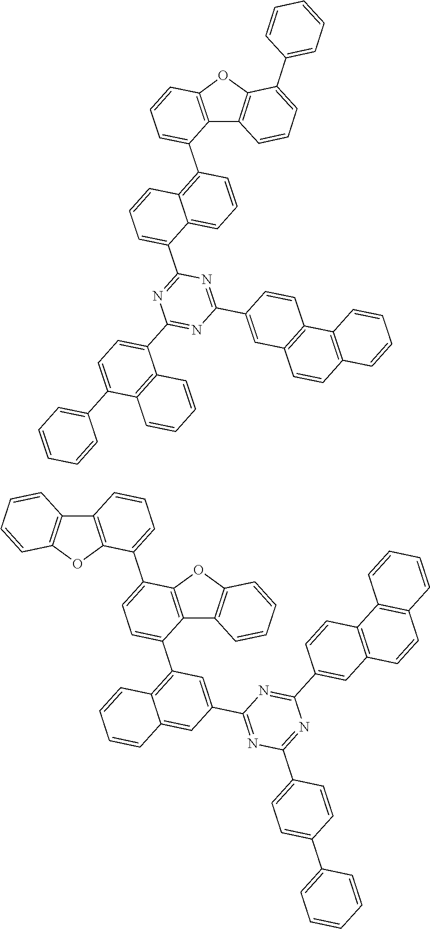

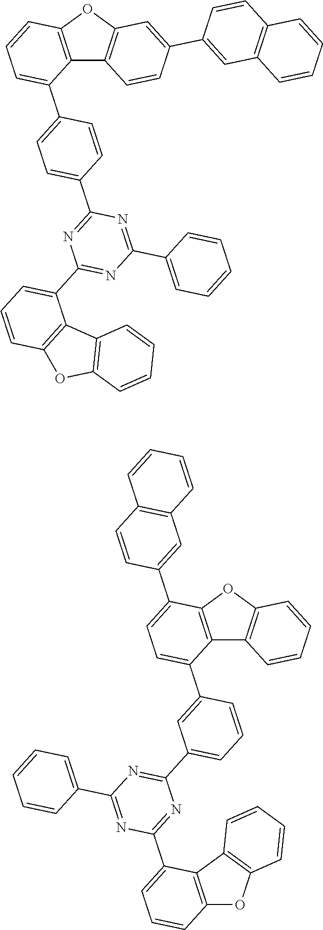 [SMILES: c1ccc(-c2nc(-c3ccc(-c4cccc5oc6cc(-c7ccc8ccccc8c7)ccc6c45)cc3)nc(-c3cccc4oc5ccccc5c34)n2)cc1 ; c1ccc(-c2nc(-c3cccc(-c4ccc(-c5ccc6ccccc6c5)c5oc6ccccc6c45)c3)nc(-c3cccc4oc5ccccc5c34)n2)cc1]

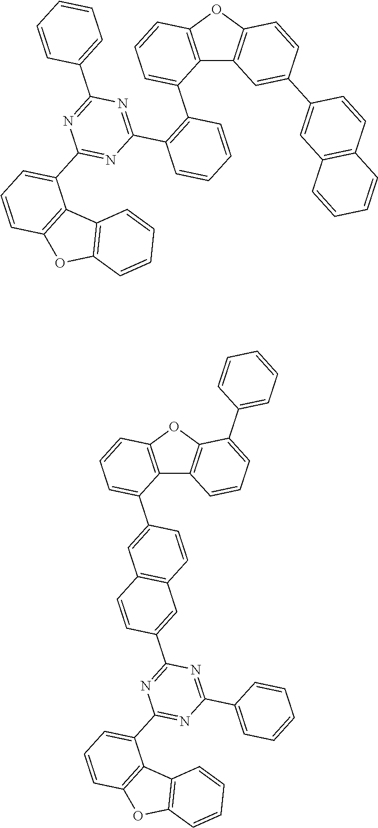 [SMILES: c1ccc(-c2nc(-c3ccccc3-c3cccc4oc5ccc(-c6ccc7ccccc7c6)cc5c34)nc(-c3cccc4oc5ccccc5c34)n2)cc1 ; c1ccc(-c2nc(-c3ccc4cc(-c5cccc6oc7c(-c8ccccc8)cccc7c56)ccc4c3)nc(-c3cccc4oc5ccccc5c34)n2)cc1]

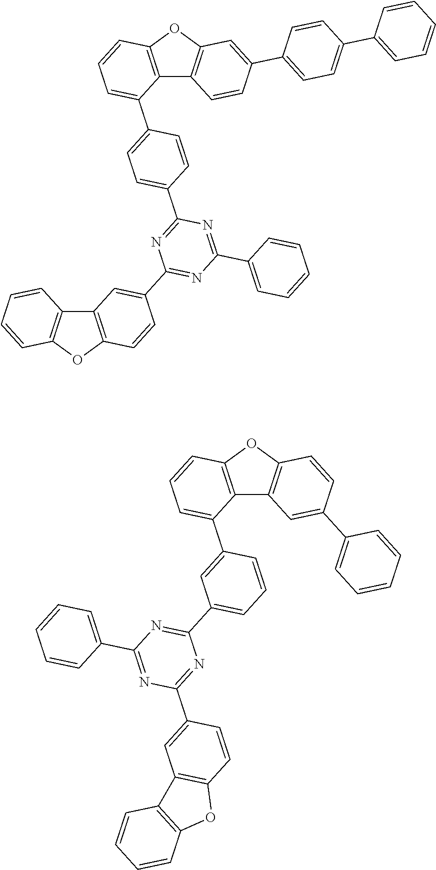 [SMILES: c1ccc(-c2ccc(-c3ccc4c(c3)oc3cccc(-c5ccc(-c6nc(-c7ccccc7)nc(-c7ccc8oc9ccccc9c8c7)n6)cc5)c34)cc2)cc1 ; c1ccc(-c2ccc3oc4cccc(-c5cccc(-c6nc(-c7ccccc7)nc(-c7ccc8oc9ccccc9c8c7)n6)c5)c4c3c2)cc1]

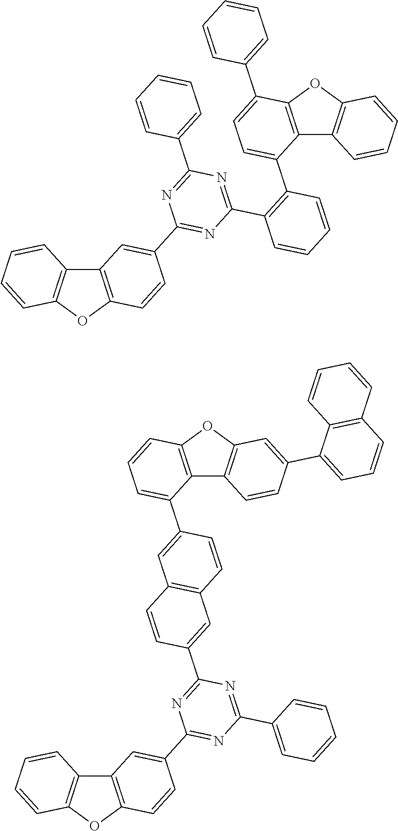 [SMILES: c1ccc(-c2nc(-c3ccc4oc5ccccc5c4c3)nc(-c3ccccc3-c3ccc(-c4ccccc4)c4oc5ccccc5c34)n2)cc1 ; c1ccc(-c2nc(-c3ccc4cc(-c5cccc6oc7cc(-c8cccc9ccccc89)ccc7c56)ccc4c3)nc(-c3ccc4oc5ccccc5c4c3)n2)cc1]

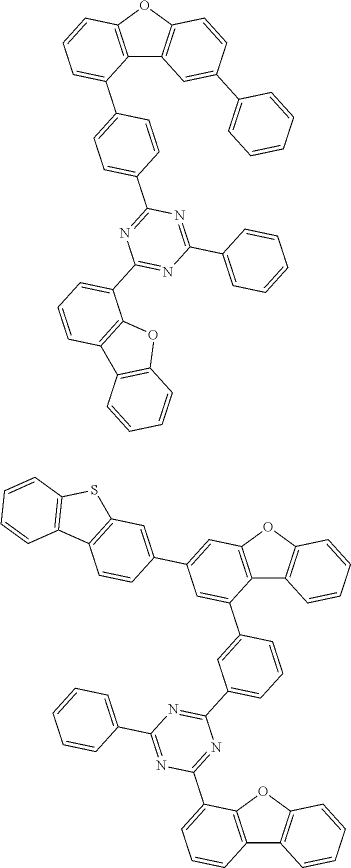 [SMILES: c1ccc(-c2ccc3oc4cccc(-c5ccc(-c6nc(-c7ccccc7)nc(-c7cccc8c7oc7ccccc78)n6)cc5)c4c3c2)cc1 ; c1ccc(-c2nc(-c3cccc(-c4cc(-c5ccc6c(c5)sc5ccccc56)cc5oc6ccccc6c45)c3)nc(-c3cccc4c3oc3ccccc34)n2)cc1]

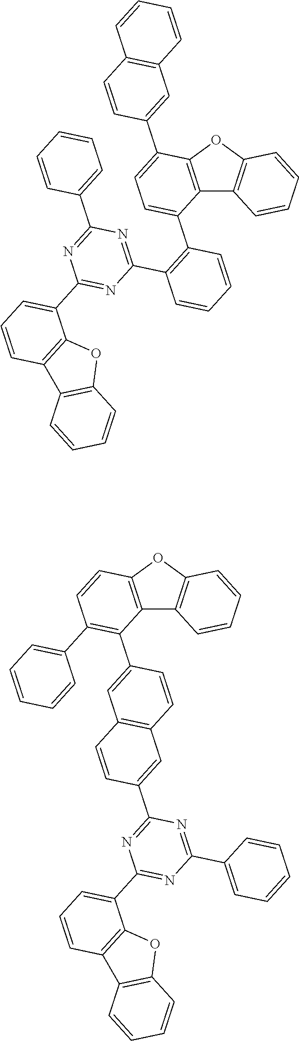 [SMILES: c1ccc(-c2nc(-c3ccccc3-c3ccc(-c4ccc5ccccc5c4)c4oc5ccccc5c34)nc(-c3cccc4c3oc3ccccc34)n2)cc1 ; c1ccc(-c2nc(-c3ccc4cc(-c5c(-c6ccccc6)ccc6oc7ccccc7c56)ccc4c3)nc(-c3cccc4c3oc3ccccc34)n2)cc1]

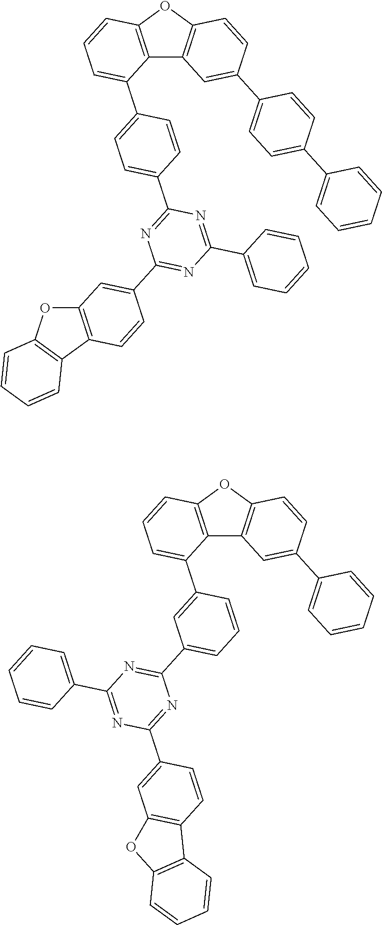 [SMILES: c1ccc(-c2ccc(-c3ccc4oc5cccc(-c6ccc(-c7nc(-c8ccccc8)nc(-c8ccc9c(c8)oc8ccccc89)n7)cc6)c5c4c3)cc2)cc1 ; c1ccc(-c2ccc3oc4cccc(-c5cccc(-c6nc(-c7ccccc7)nc(-c7ccc8c(c7)oc7ccccc78)n6)c5)c4c3c2)cc1]

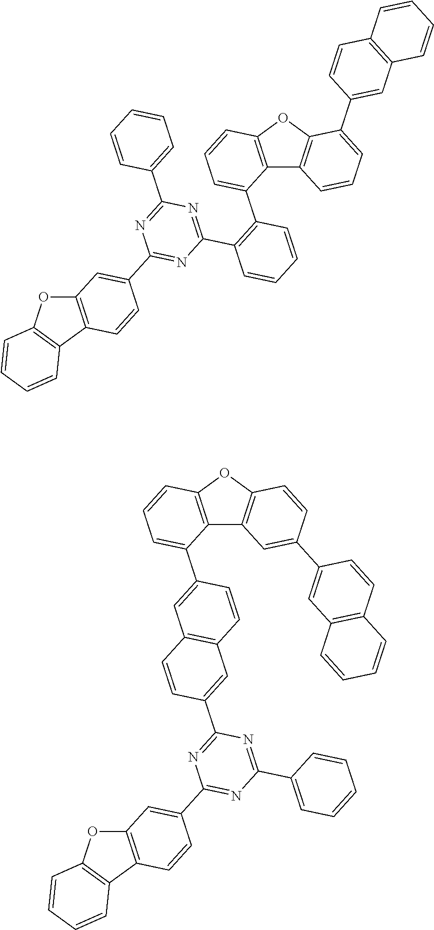 [SMILES: c1ccc(-c2nc(-c3ccc4c(c3)oc3ccccc34)nc(-c3ccccc3-c3cccc4oc5c(-c6ccc7ccccc7c6)cccc5c34)n2)cc1 ; c1ccc(-c2nc(-c3ccc4cc(-c5cccc6oc7ccc(-c8ccc9ccccc9c8)cc7c56)ccc4c3)nc(-c3ccc4c(c3)oc3ccccc34)n2)cc1]

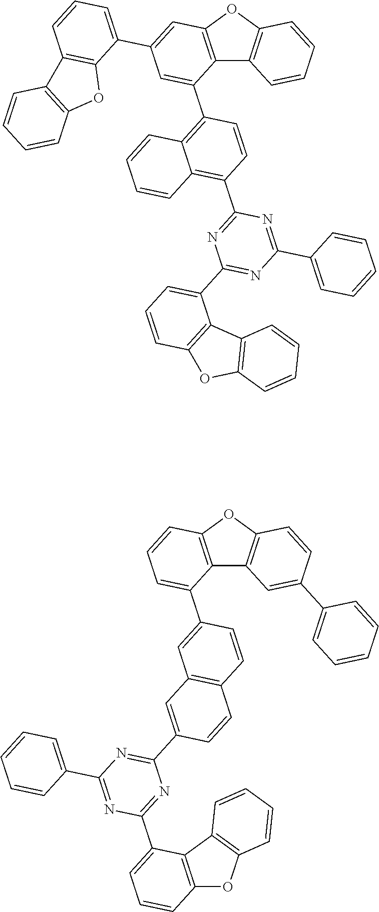 [SMILES: c1ccc(-c2nc(-c3ccc(-c4cc(-c5cccc6c5oc5ccccc56)cc5oc6ccccc6c45)c4ccccc34)nc(-c3cccc4oc5ccccc5c34)n2)cc1 ; c1ccc(-c2ccc3oc4cccc(-c5ccc6ccc(-c7nc(-c8ccccc8)nc(-c8cccc9oc%10ccccc%10c89)n7)cc6c5)c4c3c2)cc1]

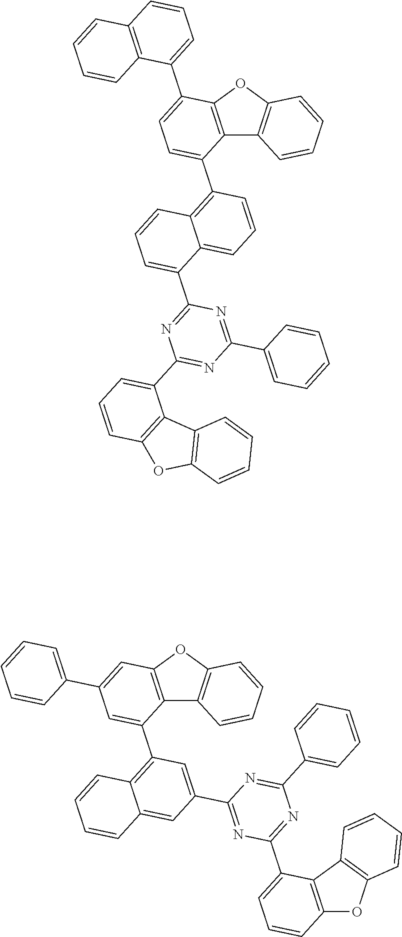 [SMILES: c1ccc(-c2nc(-c3cccc4c(-c5ccc(-c6cccc7ccccc67)c6oc7ccccc7c56)cccc34)nc(-c3cccc4oc5ccccc5c34)n2)cc1 ; c1ccc(-c2cc(-c3cc(-c4nc(-c5ccccc5)nc(-c5cccc6oc7ccccc7c56)n4)cc4ccccc34)c3c(c2)oc2ccccc23)cc1]

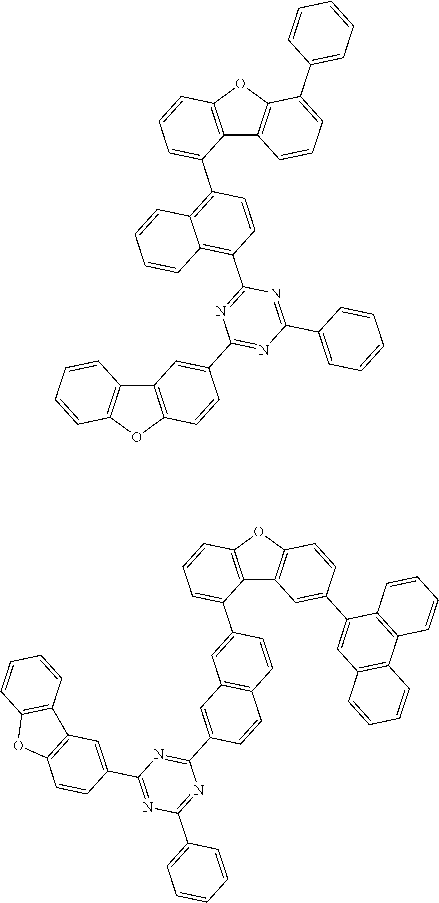 [SMILES: c1ccc(-c2nc(-c3ccc4oc5ccccc5c4c3)nc(-c3ccc(-c4cccc5oc6c(-c7ccccc7)cccc6c45)c4ccccc34)n2)cc1 ; c1ccc(-c2nc(-c3ccc4ccc(-c5cccc6oc7ccc(-c8cc9ccccc9c9ccccc89)cc7c56)cc4c3)nc(-c3ccc4oc5ccccc5c4c3)n2)cc1]

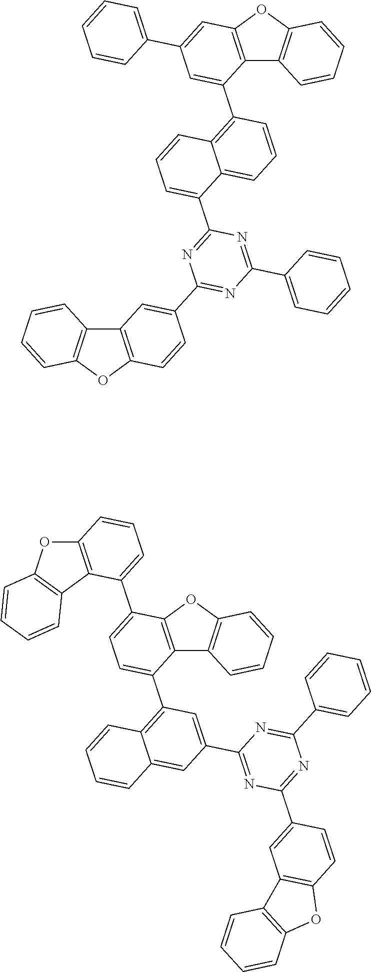 [SMILES: c1ccc(-c2cc(-c3cccc4c(-c5nc(-c6ccccc6)nc(-c6ccc7oc8ccccc8c7c6)n5)cccc34)c3c(c2)oc2ccccc23)cc1 ; c1ccc(-c2nc(-c3cc(-c4ccc(-c5cccc6oc7ccccc7c56)c5oc6ccccc6c45)c4ccccc4c3)nc(-c3ccc4oc5ccccc5c4c3)n2)cc1]

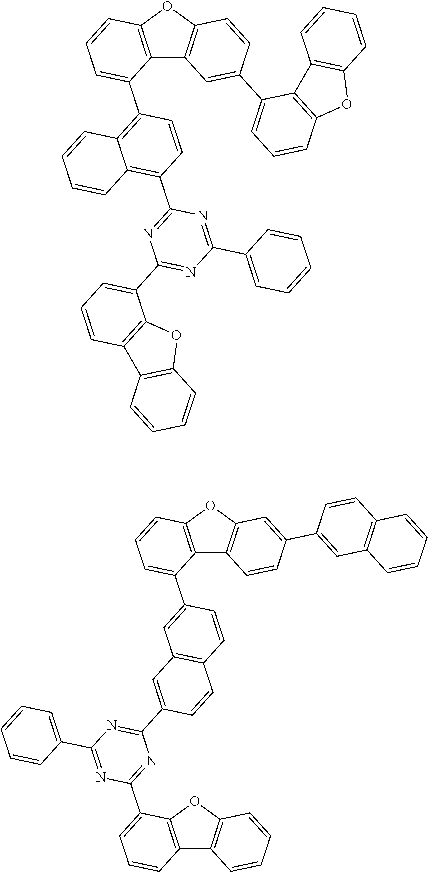 [SMILES: c1ccc(-c2nc(-c3ccc(-c4cccc5oc6ccc(-c7cccc8oc9ccccc9c78)cc6c45)c4ccccc34)nc(-c3cccc4c3oc3ccccc34)n2)cc1 ; c1ccc(-c2nc(-c3ccc4ccc(-c5cccc6oc7cc(-c8ccc9ccccc9c8)ccc7c56)cc4c3)nc(-c3cccc4c3oc3ccccc34)n2)cc1]

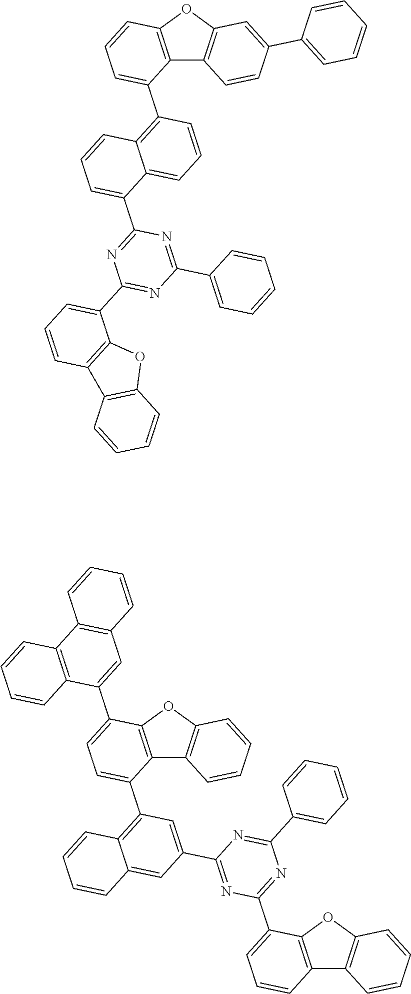 [SMILES: c1ccc(-c2ccc3c(c2)oc2cccc(-c4cccc5c(-c6nc(-c7ccccc7)nc(-c7cccc8c7oc7ccccc78)n6)cccc45)c23)cc1 ; c1ccc(-c2nc(-c3cc(-c4ccc(-c5cc6ccccc6c6ccccc56)c5oc6ccccc6c45)c4ccccc4c3)nc(-c3cccc4c3oc3ccccc34)n2)cc1]

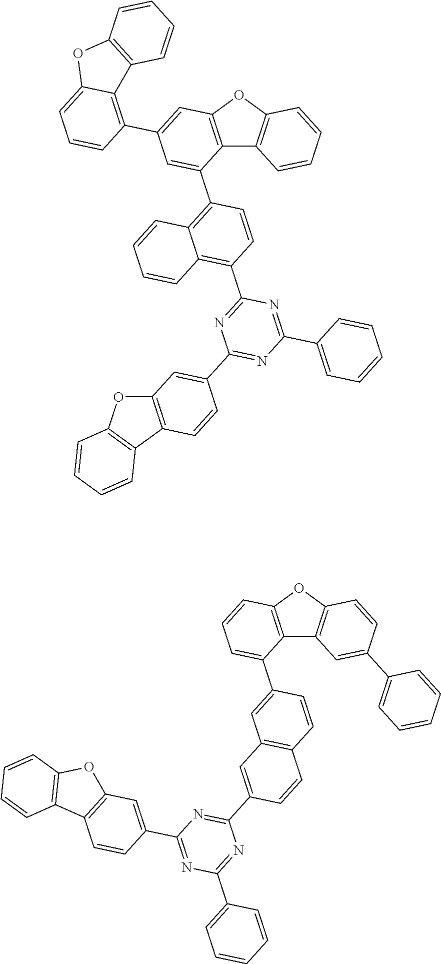 [SMILES: c1ccc(-c2nc(-c3ccc4c(c3)oc3ccccc34)nc(-c3ccc(-c4cc(-c5cccc6oc7ccccc7c56)cc5oc6ccccc6c45)c4ccccc34)n2)cc1 ; c1ccc(-c2ccc3oc4cccc(-c5ccc6ccc(-c7nc(-c8ccccc8)nc(-c8ccc9c(c8)oc8ccccc89)n7)cc6c5)c4c3c2)cc1]

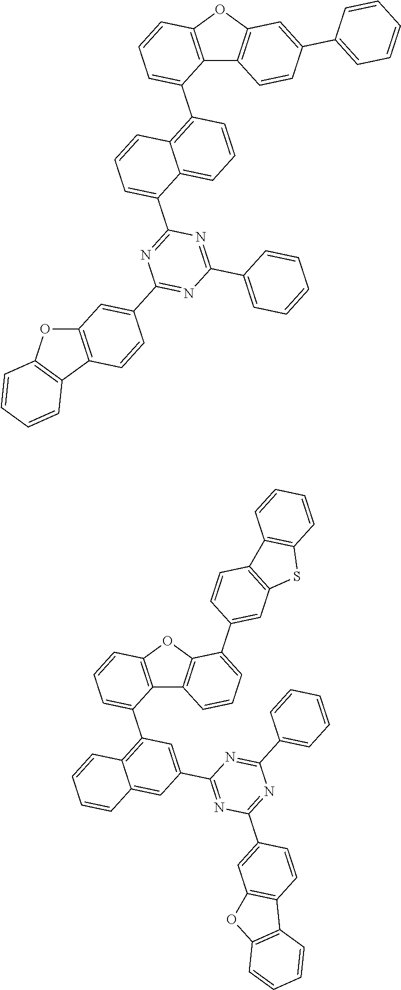 [SMILES: c1ccc(-c2ccc3c(c2)oc2cccc(-c4cccc5c(-c6nc(-c7ccccc7)nc(-c7ccc8c(c7)oc7ccccc78)n6)cccc45)c23)cc1 ; c1ccc(-c2nc(-c3cc(-c4cccc5oc6c(-c7ccc8c(c7)sc7ccccc78)cccc6c45)c4ccccc4c3)nc(-c3ccc4c(c3)oc3ccccc34)n2)cc1]

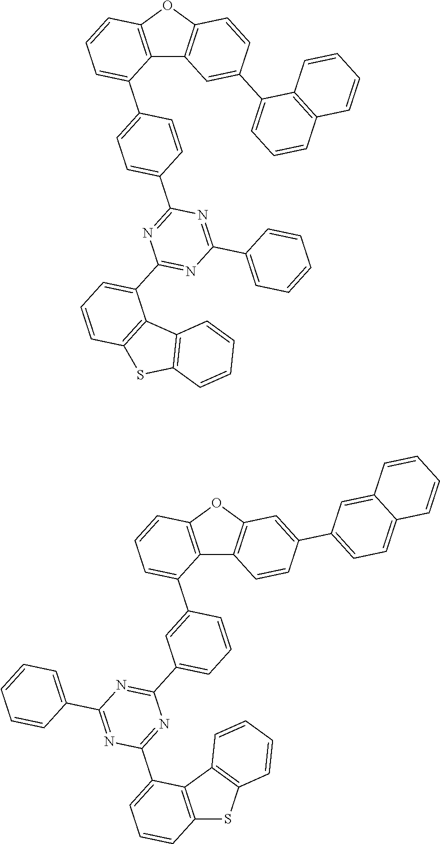 [SMILES: c1ccc(-c2nc(-c3ccc(-c4cccc5oc6ccc(-c7cccc8ccccc78)cc6c45)cc3)nc(-c3cccc4sc5ccccc5c34)n2)cc1 ; c1ccc(-c2nc(-c3cccc(-c4cccc5oc6cc(-c7ccc8ccccc8c7)ccc6c45)c3)nc(-c3cccc4sc5ccccc5c34)n2)cc1]

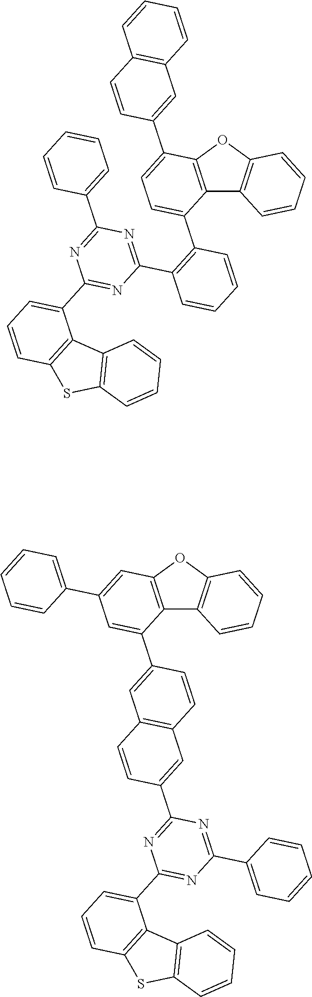 [SMILES: c1ccc(-c2nc(-c3ccccc3-c3ccc(-c4ccc5ccccc5c4)c4oc5ccccc5c34)nc(-c3cccc4sc5ccccc5c34)n2)cc1 ; c1ccc(-c2cc(-c3ccc4cc(-c5nc(-c6ccccc6)nc(-c6cccc7sc8ccccc8c67)n5)ccc4c3)c3c(c2)oc2ccccc23)cc1]

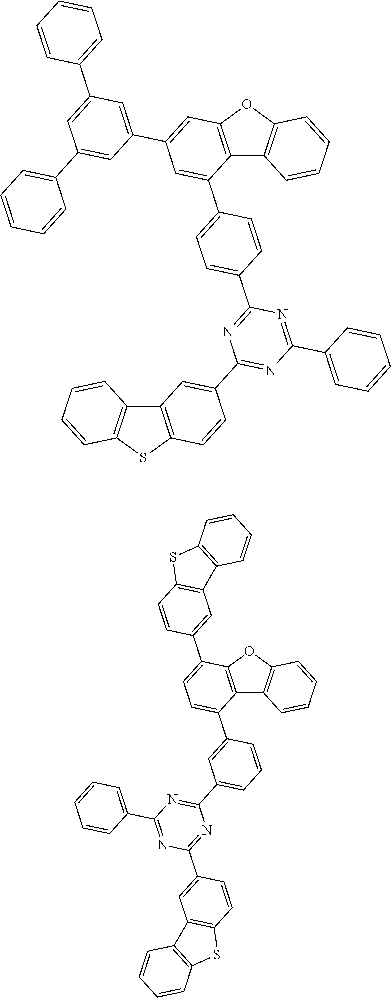 [SMILES: c1ccc(-c2cc(-c3ccccc3)cc(-c3cc(-c4ccc(-c5nc(-c6ccccc6)nc(-c6ccc7sc8ccccc8c7c6)n5)cc4)c4c(c3)oc3ccccc34)c2)cc1 ; c1ccc(-c2nc(-c3cccc(-c4ccc(-c5ccc6sc7ccccc7c6c5)c5oc6ccccc6c45)c3)nc(-c3ccc4sc5ccccc5c4c3)n2)cc1]

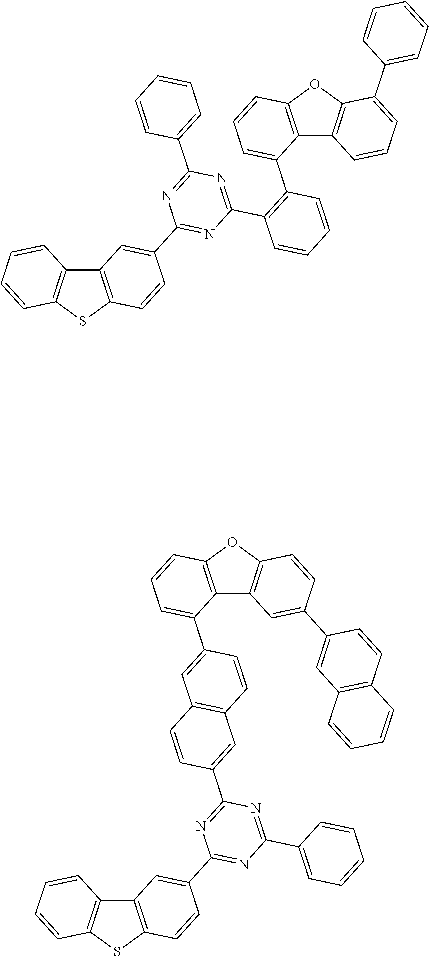 [SMILES: c1ccc(-c2nc(-c3ccc4sc5ccccc5c4c3)nc(-c3ccccc3-c3cccc4oc5c(-c6ccccc6)cccc5c34)n2)cc1 ; c1ccc(-c2nc(-c3ccc4cc(-c5cccc6oc7ccc(-c8ccc9ccccc9c8)cc7c56)ccc4c3)nc(-c3ccc4sc5ccccc5c4c3)n2)cc1]

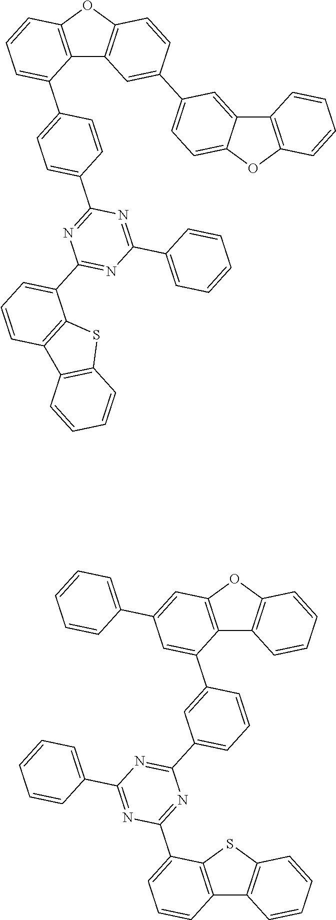 [SMILES: c1ccc(-c2nc(-c3ccc(-c4cccc5oc6ccc(-c7ccc8oc9ccccc9c8c7)cc6c45)cc3)nc(-c3cccc4c3sc3ccccc34)n2)cc1 ; c1ccc(-c2cc(-c3cccc(-c4nc(-c5ccccc5)nc(-c5cccc6c5sc5ccccc56)n4)c3)c3c(c2)oc2ccccc23)cc1]

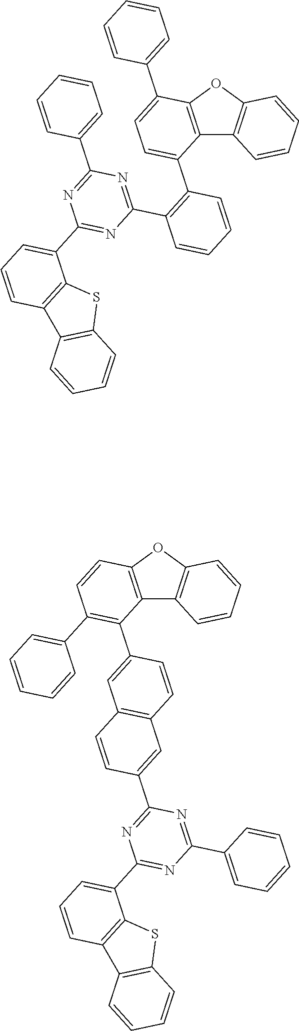 [SMILES: c1ccc(-c2nc(-c3ccccc3-c3ccc(-c4ccccc4)c4oc5ccccc5c34)nc(-c3cccc4c3sc3ccccc34)n2)cc1 ; c1ccc(-c2nc(-c3ccc4cc(-c5c(-c6ccccc6)ccc6oc7ccccc7c56)ccc4c3)nc(-c3cccc4c3sc3ccccc34)n2)cc1]

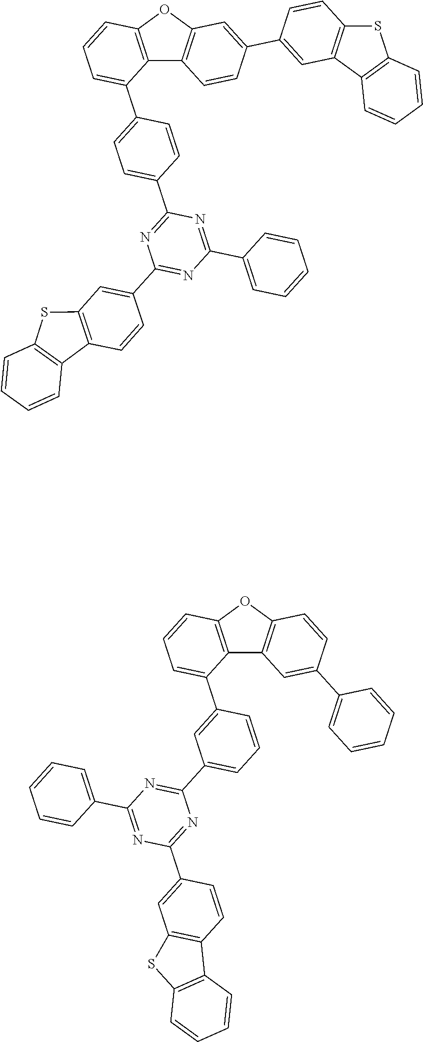 [SMILES: c1ccc(-c2nc(-c3ccc(-c4cccc5oc6cc(-c7ccc8sc9ccccc9c8c7)ccc6c45)cc3)nc(-c3ccc4c(c3)sc3ccccc34)n2)cc1 ; c1ccc(-c2ccc3oc4cccc(-c5cccc(-c6nc(-c7ccccc7)nc(-c7ccc8c(c7)sc7ccccc78)n6)c5)c4c3c2)cc1]

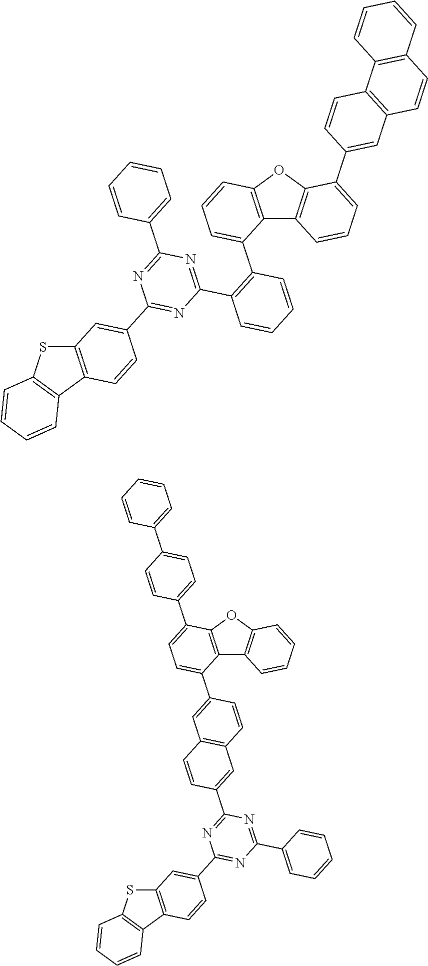 [SMILES: c1ccc(-c2nc(-c3ccc4c(c3)sc3ccccc34)nc(-c3ccccc3-c3cccc4oc5c(-c6ccc7c(ccc8ccccc87)c6)cccc5c34)n2)cc1 ; c1ccc(-c2ccc(-c3ccc(-c4ccc5cc(-c6nc(-c7ccccc7)nc(-c7ccc8c(c7)sc7ccccc78)n6)ccc5c4)c4c3oc3ccccc34)cc2)cc1]

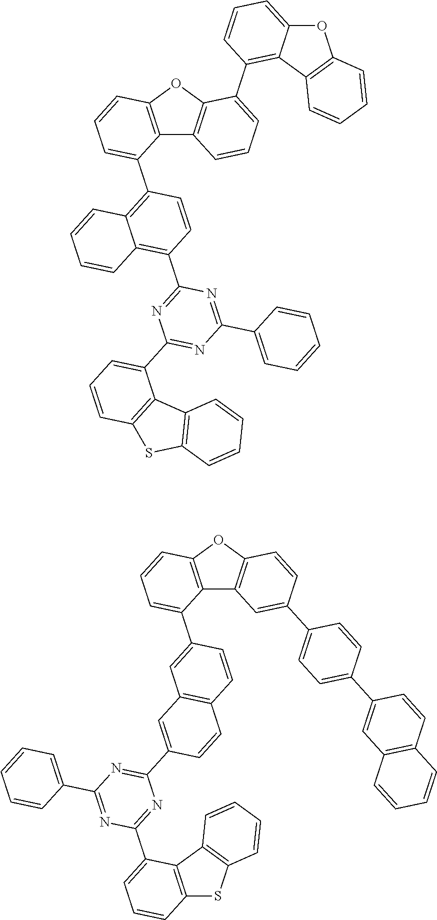 [SMILES: c1ccc(-c2nc(-c3ccc(-c4cccc5oc6c(-c7cccc8oc9ccccc9c78)cccc6c45)c4ccccc34)nc(-c3cccc4sc5ccccc5c34)n2)cc1 ; c1ccc(-c2nc(-c3ccc4ccc(-c5cccc6oc7ccc(-c8ccc(-c9ccc%10ccccc%10c9)cc8)cc7c56)cc4c3)nc(-c3cccc4sc5ccccc5c34)n2)cc1]

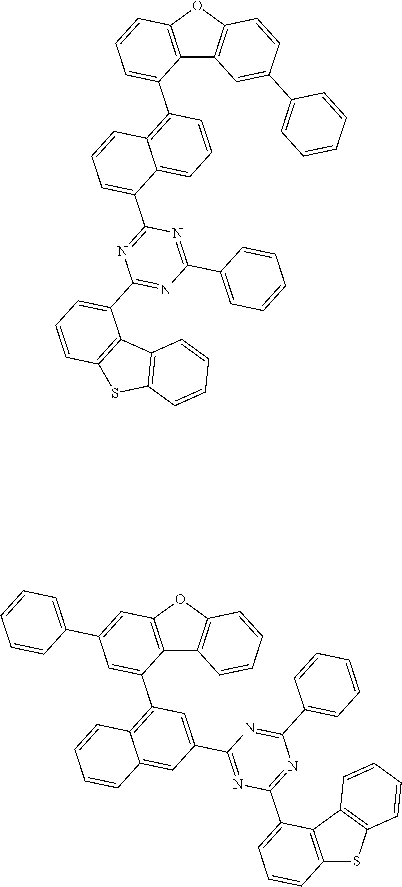 [SMILES: c1ccc(-c2ccc3oc4cccc(-c5cccc6c(-c7nc(-c8ccccc8)nc(-c8cccc9sc%10ccccc%10c89)n7)cccc56)c4c3c2)cc1 ; c1ccc(-c2cc(-c3cc(-c4nc(-c5ccccc5)nc(-c5cccc6sc7ccccc7c56)n4)cc4ccccc34)c3c(c2)oc2ccccc23)cc1]

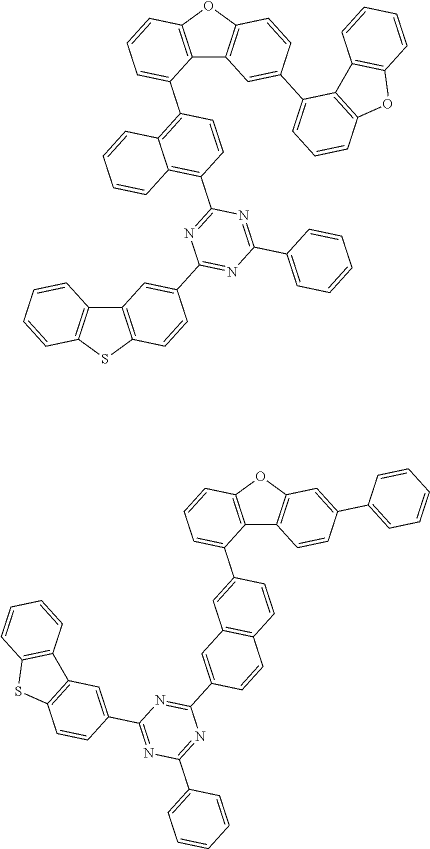 [SMILES: c1ccc(-c2nc(-c3ccc4sc5ccccc5c4c3)nc(-c3ccc(-c4cccc5oc6ccc(-c7cccc8oc9ccccc9c78)cc6c45)c4ccccc34)n2)cc1 ; c1ccc(-c2ccc3c(c2)oc2cccc(-c4ccc5ccc(-c6nc(-c7ccccc7)nc(-c7ccc8sc9ccccc9c8c7)n6)cc5c4)c23)cc1]

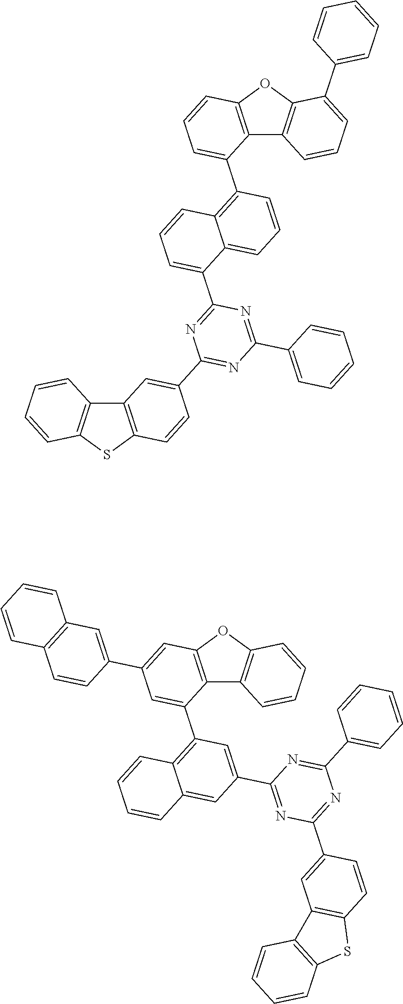 [SMILES: c1ccc(-c2nc(-c3ccc4sc5ccccc5c4c3)nc(-c3cccc4c(-c5cccc6oc7c(-c8ccccc8)cccc7c56)cccc34)n2)cc1 ; c1ccc(-c2nc(-c3cc(-c4cc(-c5ccc6ccccc6c5)cc5oc6ccccc6c45)c4ccccc4c3)nc(-c3ccc4sc5ccccc5c4c3)n2)cc1]

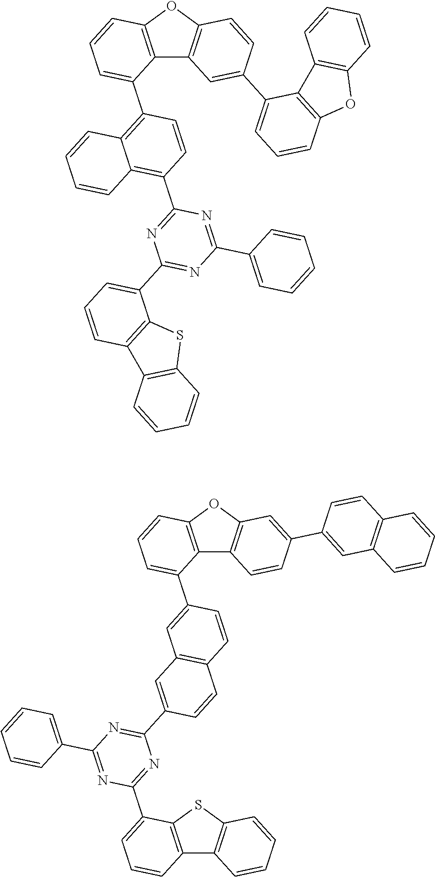 [SMILES: c1ccc(-c2nc(-c3ccc(-c4cccc5oc6ccc(-c7cccc8oc9ccccc9c78)cc6c45)c4ccccc34)nc(-c3cccc4c3sc3ccccc34)n2)cc1 ; c1ccc(-c2nc(-c3ccc4ccc(-c5cccc6oc7cc(-c8ccc9ccccc9c8)ccc7c56)cc4c3)nc(-c3cccc4c3sc3ccccc34)n2)cc1]

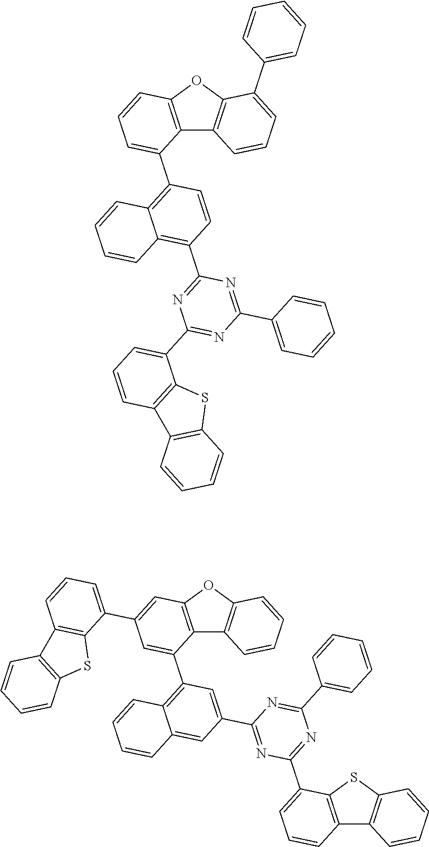 [SMILES: c1ccc(-c2nc(-c3ccc(-c4cccc5oc6c(-c7ccccc7)cccc6c45)c4ccccc34)nc(-c3cccc4c3sc3ccccc34)n2)cc1 ; c1ccc(-c2nc(-c3cc(-c4cc(-c5cccc6c5sc5ccccc56)cc5oc6ccccc6c45)c4ccccc4c3)nc(-c3cccc4c3sc3ccccc34)n2)cc1]

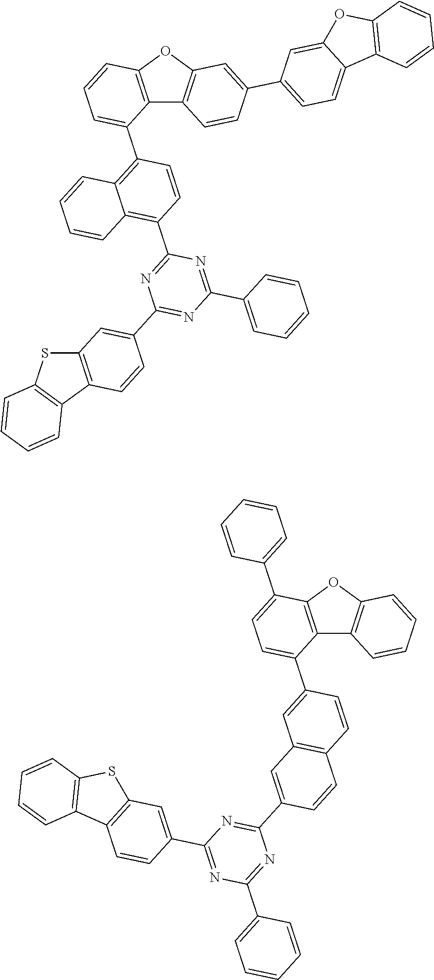 [SMILES: c1ccc(-c2nc(-c3ccc4c(c3)sc3ccccc34)nc(-c3ccc(-c4cccc5oc6cc(-c7ccc8c(c7)oc7ccccc78)ccc6c45)c4ccccc34)n2)cc1 ; c1ccc(-c2nc(-c3ccc4ccc(-c5ccc(-c6ccccc6)c6oc7ccccc7c56)cc4c3)nc(-c3ccc4c(c3)sc3ccccc34)n2)cc1]

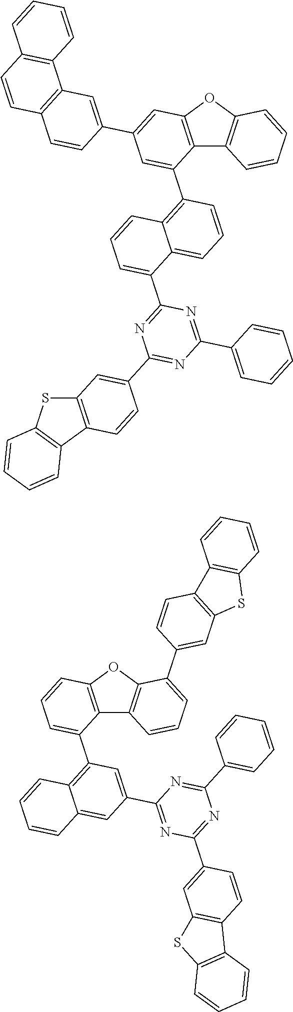 [SMILES: c1ccc(-c2nc(-c3ccc4c(c3)sc3ccccc34)nc(-c3cccc4c(-c5cc(-c6ccc7ccc8ccccc8c7c6)cc6oc7ccccc7c56)cccc34)n2)cc1 ; c1ccc(-c2nc(-c3cc(-c4cccc5oc6c(-c7ccc8c(c7)sc7ccccc78)cccc6c45)c4ccccc4c3)nc(-c3ccc4c(c3)sc3ccccc34)n2)cc1]

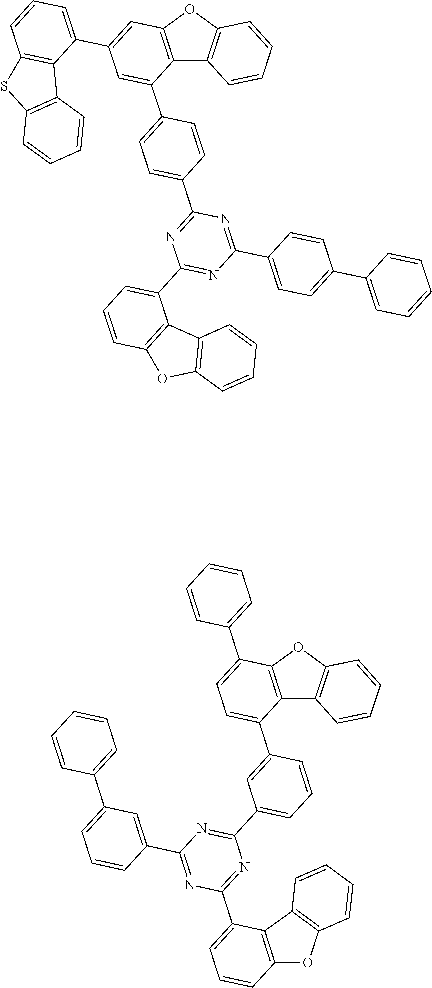 [SMILES: c1ccc(-c2ccc(-c3nc(-c4ccc(-c5cc(-c6cccc7sc8ccccc8c67)cc6oc7ccccc7c56)cc4)nc(-c4cccc5oc6ccccc6c45)n3)cc2)cc1 ; c1ccc(-c2cccc(-c3nc(-c4cccc(-c5ccc(-c6ccccc6)c6oc7ccccc7c56)c4)nc(-c4cccc5oc6ccccc6c45)n3)c2)cc1]

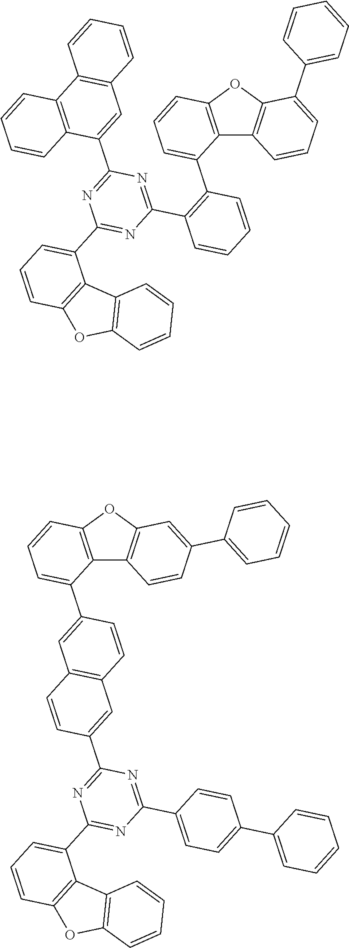 [SMILES: c1ccc(-c2cccc3c2oc2cccc(-c4ccccc4-c4nc(-c5cc6ccccc6c6ccccc56)nc(-c5cccc6oc7ccccc7c56)n4)c23)cc1 ; c1ccc(-c2ccc(-c3nc(-c4ccc5cc(-c6cccc7oc8cc(-c9ccccc9)ccc8c67)ccc5c4)nc(-c4cccc5oc6ccccc6c45)n3)cc2)cc1]

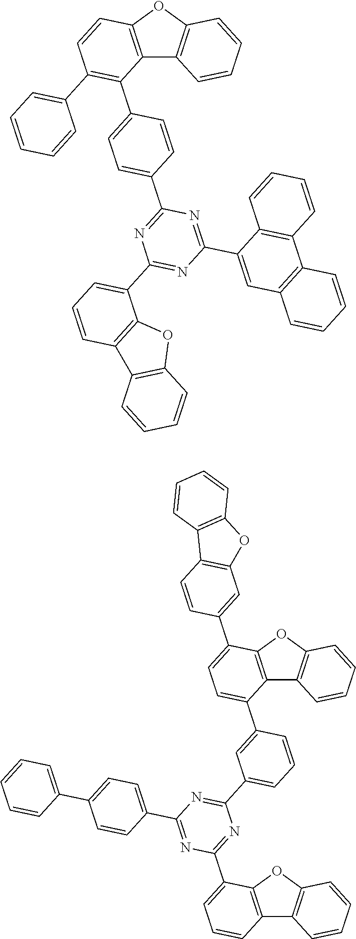

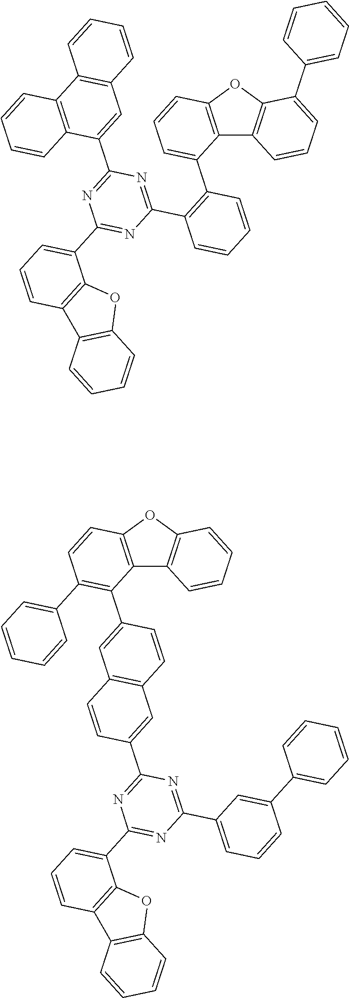

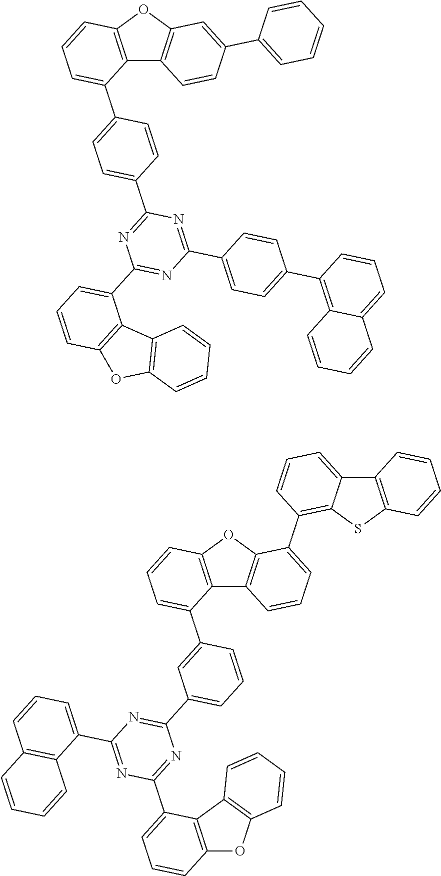 [SMILES: c1ccc(-c2ccc3c(c2)oc2cccc(-c4ccc(-c5nc(-c6ccc(-c7cccc8ccccc78)cc6)nc(-c6cccc7oc8ccccc8c67)n5)cc4)c23)cc1 ; c1cc(-c2nc(-c3cccc4ccccc34)nc(-c3cccc4oc5ccccc5c34)n2)cc(-c2cccc3oc4c(-c5cccc6c5sc5ccccc56)cccc4c23)c1]

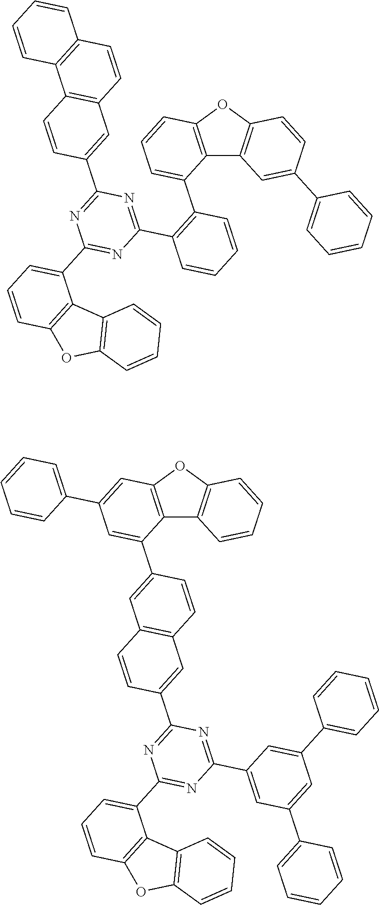 [SMILES: c1ccc(-c2ccc3oc4cccc(-c5ccccc5-c5nc(-c6ccc7c(ccc8ccccc87)c6)nc(-c6cccc7oc8ccccc8c67)n5)c4c3c2)cc1 ; c1ccc(-c2cc(-c3ccccc3)cc(-c3nc(-c4ccc5cc(-c6cc(-c7ccccc7)cc7oc8ccccc8c67)ccc5c4)nc(-c4cccc5oc6ccccc6c45)n3)c2)cc1]

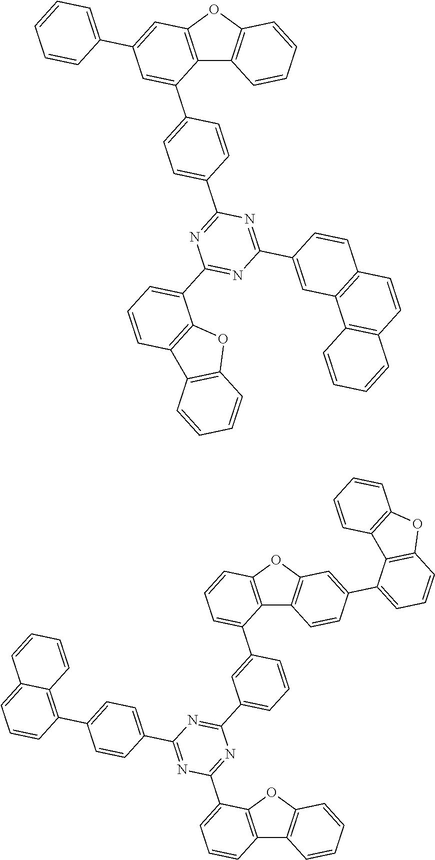 [SMILES: c1ccc(-c2cc(-c3ccc(-c4nc(-c5ccc6ccc7ccccc7c6c5)nc(-c5cccc6c5oc5ccccc56)n4)cc3)c3c(c2)oc2ccccc23)cc1 ; c1cc(-c2nc(-c3ccc(-c4cccc5ccccc45)cc3)nc(-c3cccc4c3oc3ccccc34)n2)cc(-c2cccc3oc4cc(-c5cccc6oc7ccccc7c56)ccc4c23)c1]

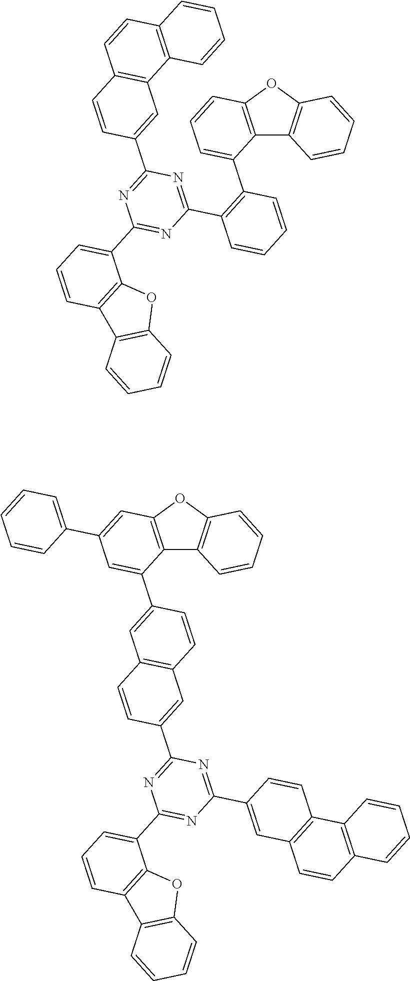 [SMILES: c1ccc(-c2cccc3oc4ccccc4c23)c(-c2nc(-c3ccc4ccc5ccccc5c4c3)nc(-c3cccc4c3oc3ccccc34)n2)c1 ; c1ccc(-c2cc(-c3ccc4cc(-c5nc(-c6ccc7c(ccc8ccccc87)c6)nc(-c6cccc7c6oc6ccccc67)n5)ccc4c3)c3c(c2)oc2ccccc23)cc1]

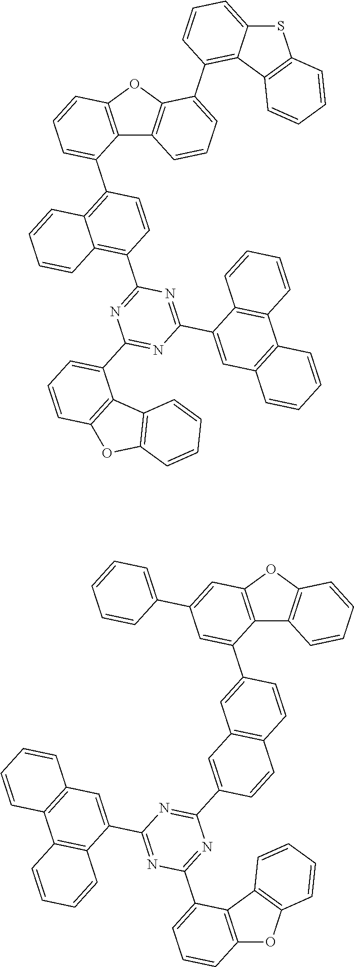 [SMILES: c1ccc2c(c1)cc(-c1nc(-c3ccc(-c4cccc5oc6c(-c7cccc8sc9ccccc9c78)cccc6c45)c4ccccc34)nc(-c3cccc4oc5ccccc5c34)n1)c1ccccc12 ; c1ccc(-c2cc(-c3ccc4ccc(-c5nc(-c6cc7ccccc7c7ccccc67)nc(-c6cccc7oc8ccccc8c67)n5)cc4c3)c3c(c2)oc2ccccc23)cc1]

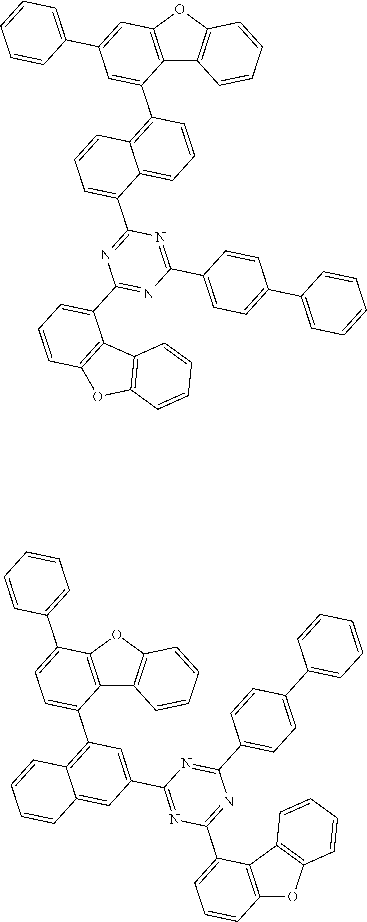 [SMILES: c1ccc(-c2ccc(-c3nc(-c4cccc5c(-c6cc(-c7ccccc7)cc7oc8ccccc8c67)cccc45)nc(-c4cccc5oc6ccccc6c45)n3)cc2)cc1 ; c1ccc(-c2ccc(-c3nc(-c4cc(-c5ccc(-c6ccccc6)c6oc7ccccc7c56)c5ccccc5c4)nc(-c4cccc5oc6ccccc6c45)n3)cc2)cc1]

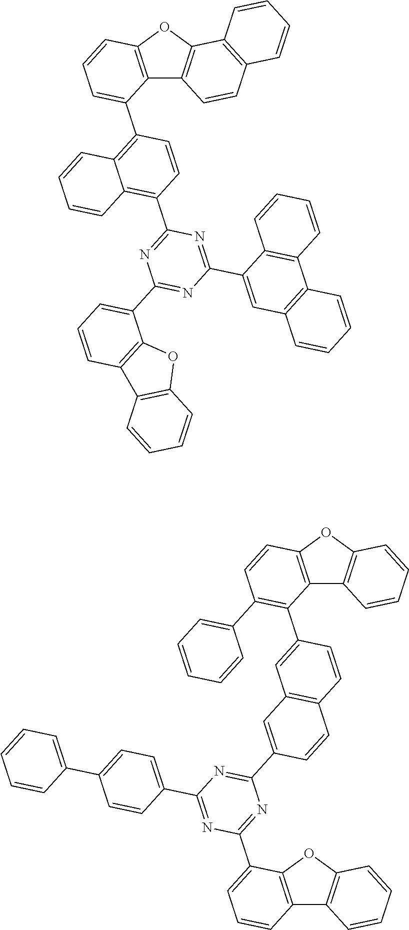 [SMILES: c1ccc2c(c1)cc(-c1nc(-c3ccc(-c4cccc5oc6c7ccccc7ccc6c45)c4ccccc34)nc(-c3cccc4c3oc3ccccc34)n1)c1ccccc12 ; c1ccc(-c2ccc(-c3nc(-c4ccc5ccc(-c6c(-c7ccccc7)ccc7oc8ccccc8c67)cc5c4)nc(-c4cccc5c4oc4ccccc45)n3)cc2)cc1]

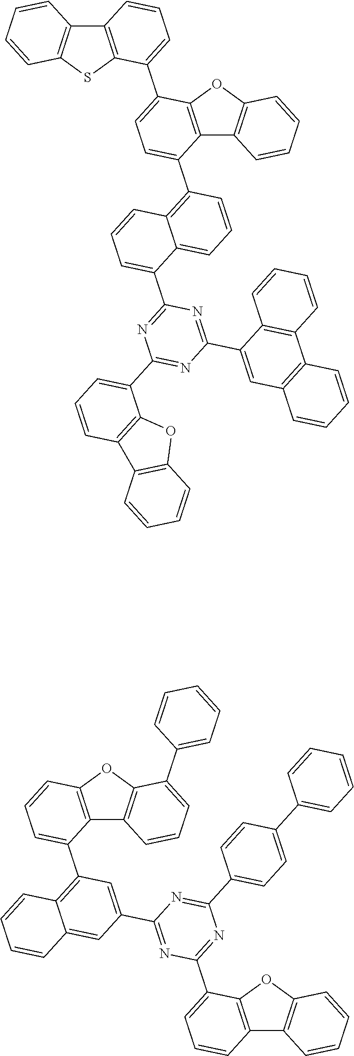 [SMILES: c1ccc2c(c1)cc(-c1nc(-c3cccc4c(-c5ccc(-c6cccc7c6sc6ccccc67)c6oc7ccccc7c56)cccc34)nc(-c3cccc4c3oc3ccccc34)n1)c1ccccc12 ; c1ccc(-c2ccc(-c3nc(-c4cc(-c5cccc6oc7c(-c8ccccc8)cccc7c56)c5ccccc5c4)nc(-c4cccc5c4oc4ccccc45)n3)cc2)cc1]

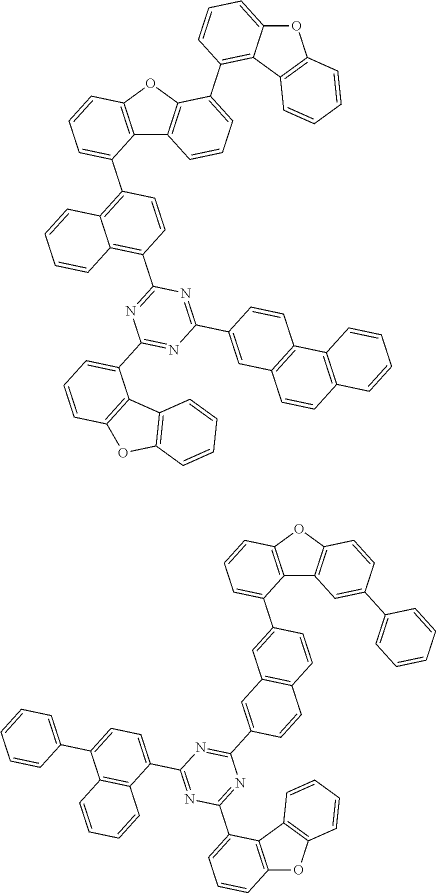

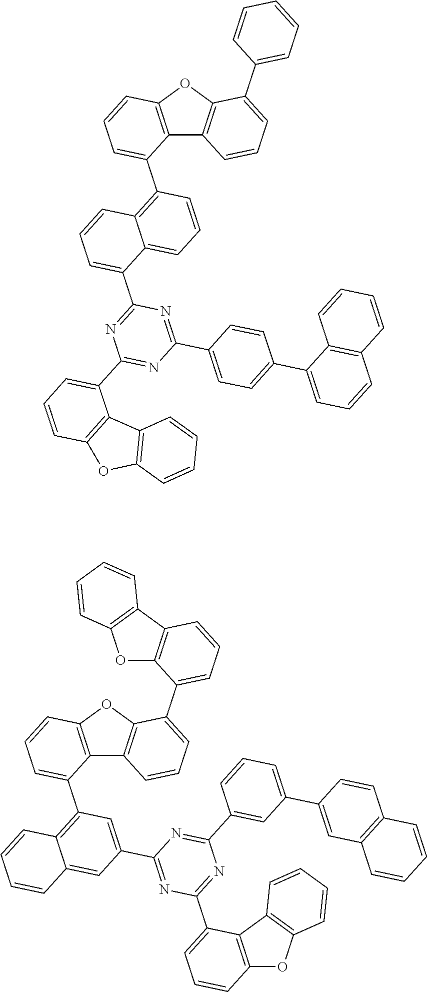

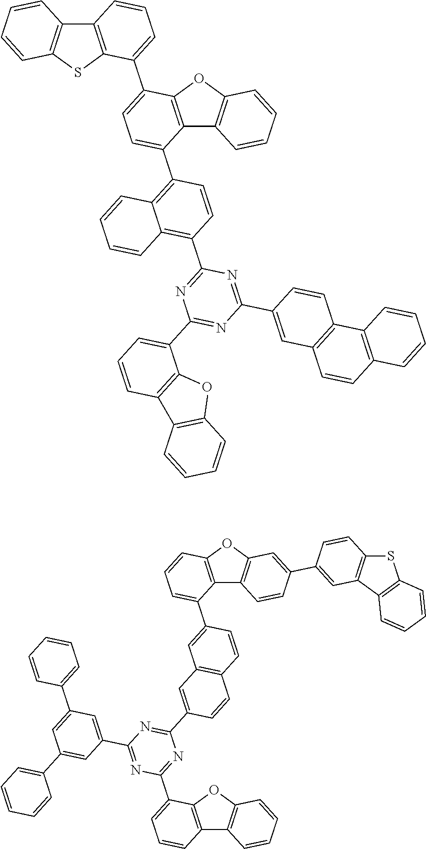 [SMILES: c1ccc2c(c1)ccc1cc(-c3nc(-c4ccc(-c5ccc(-c6cccc7c6sc6ccccc67)c6oc7ccccc7c56)c5ccccc45)nc(-c4cccc5c4oc4ccccc45)n3)ccc12 ; c1ccc(-c2cc(-c3ccccc3)cc(-c3nc(-c4ccc5ccc(-c6cccc7oc8cc(-c9ccc%10sc%11ccccc%11c%10c9)ccc8c67)cc5c4)nc(-c4cccc5c4oc4ccccc45)n3)c2)cc1]

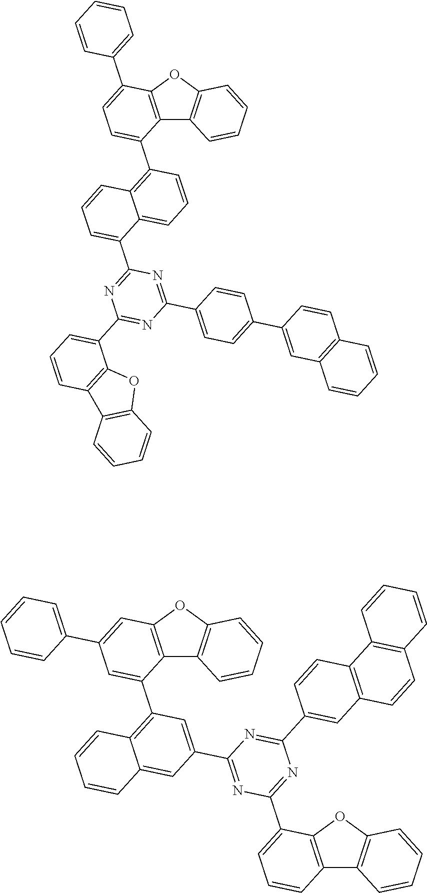 [SMILES: c1ccc(-c2ccc(-c3cccc4c(-c5nc(-c6ccc(-c7ccc8ccccc8c7)cc6)nc(-c6cccc7c6oc6ccccc67)n5)cccc34)c3c2oc2ccccc23)cc1 ; c1ccc(-c2cc(-c3cc(-c4nc(-c5ccc6c(ccc7ccccc76)c5)nc(-c5cccc6c5oc5ccccc56)n4)cc4ccccc34)c3c(c2)oc2ccccc23)cc1]

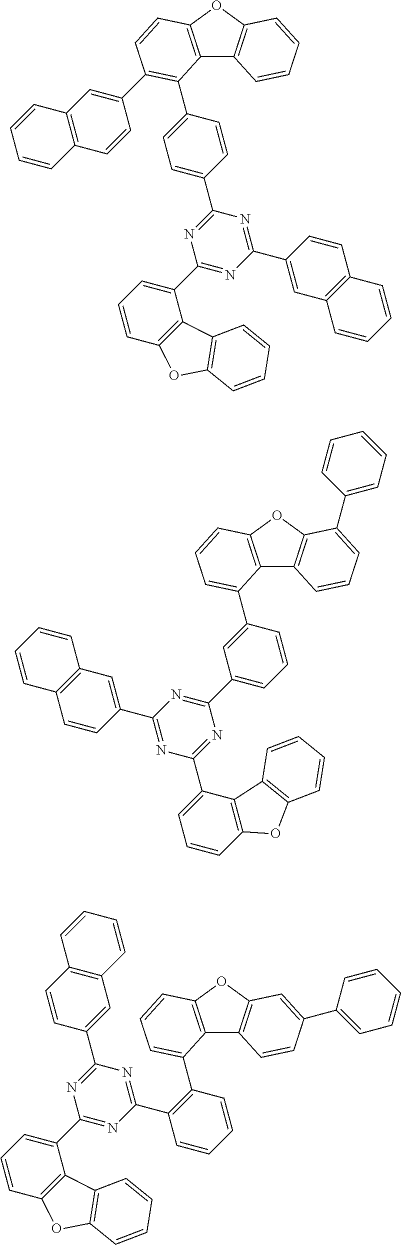 [SMILES: c1ccc2cc(-c3nc(-c4ccc(-c5c(-c6ccc7ccccc7c6)ccc6oc7ccccc7c56)cc4)nc(-c4cccc5oc6ccccc6c45)n3)ccc2c1 ; c1ccc(-c2cccc3c2oc2cccc(-c4cccc(-c5nc(-c6ccc7ccccc7c6)nc(-c6cccc7oc8ccccc8c67)n5)c4)c23)cc1 ; c1ccc(-c2ccc3c(c2)oc2cccc(-c4ccccc4-c4nc(-c5ccc6ccccc6c5)nc(-c5cccc6oc7ccccc7c56)n4)c23)cc1]

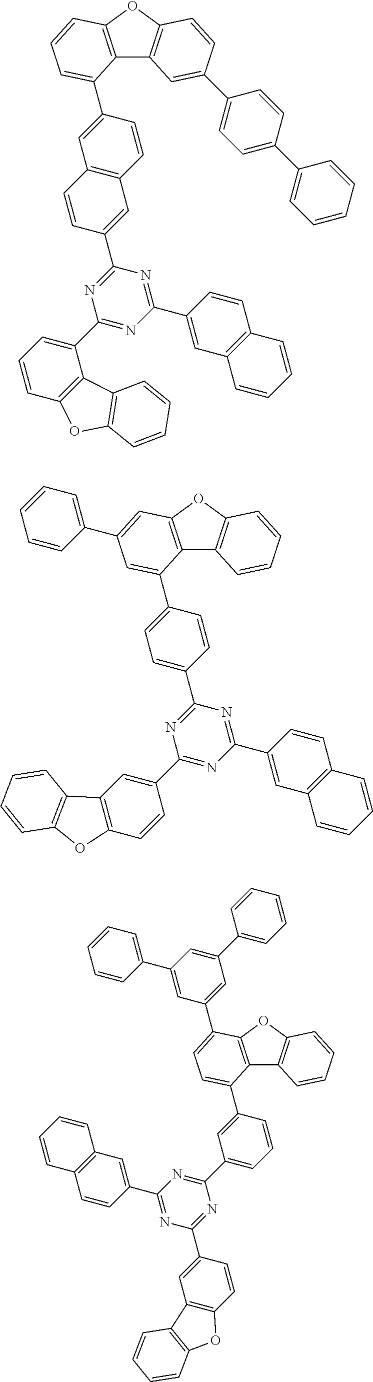 [SMILES: c1ccc(-c2ccc(-c3ccc4oc5cccc(-c6ccc7cc(-c8nc(-c9ccc%10ccccc%10c9)nc(-c9cccc%10oc%11ccccc%11c9%10)n8)ccc7c6)c5c4c3)cc2)cc1 ; c1ccc(-c2cc(-c3ccc(-c4nc(-c5ccc6ccccc6c5)nc(-c5ccc6oc7ccccc7c6c5)n4)cc3)c3c(c2)oc2ccccc23)cc1 ; c1ccc(-c2cc(-c3ccccc3)cc(-c3ccc(-c4cccc(-c5nc(-c6ccc7ccccc7c6)nc(-c6ccc7oc8ccccc8c7c6)n5)c4)c4c3oc3ccccc34)c2)cc1]

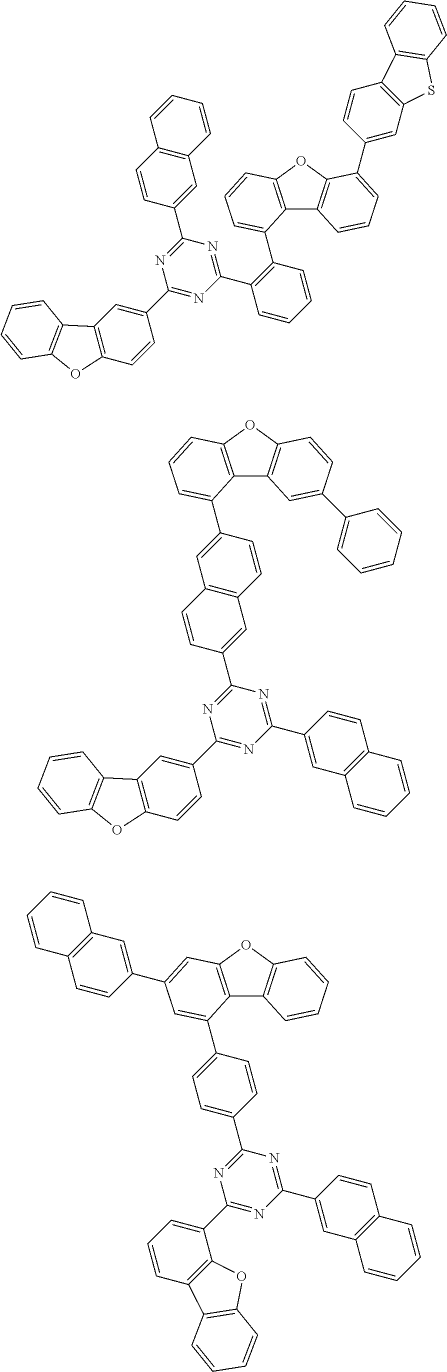 [SMILES: c1ccc(-c2cccc3oc4c(-c5ccc6c(c5)sc5ccccc56)cccc4c23)c(-c2nc(-c3ccc4ccccc4c3)nc(-c3ccc4oc5ccccc5c4c3)n2)c1 ; c1ccc(-c2ccc3oc4cccc(-c5ccc6cc(-c7nc(-c8ccc9ccccc9c8)nc(-c8ccc9oc%10ccccc%10c9c8)n7)ccc6c5)c4c3c2)cc1 ; c1ccc2cc(-c3cc(-c4ccc(-c5nc(-c6ccc7ccccc7c6)nc(-c6cccc7c6oc6ccccc67)n5)cc4)c4c(c3)oc3ccccc34)ccc2c1]

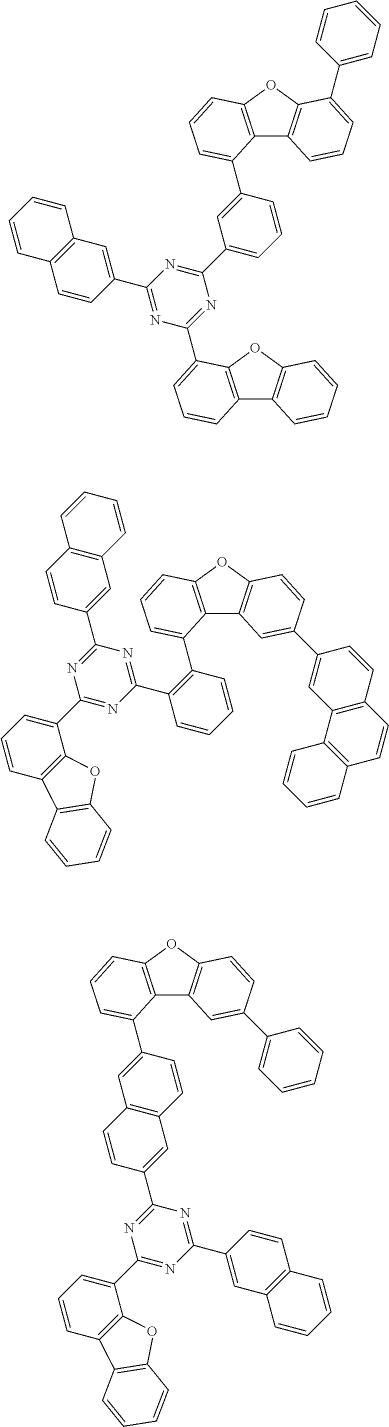 [SMILES: c1ccc(-c2cccc3c2oc2cccc(-c4cccc(-c5nc(-c6ccc7ccccc7c6)nc(-c6cccc7c6oc6ccccc67)n5)c4)c23)cc1 ; c1ccc(-c2cccc3oc4ccc(-c5ccc6ccc7ccccc7c6c5)cc4c23)c(-c2nc(-c3ccc4ccccc4c3)nc(-c3cccc4c3oc3ccccc34)n2)c1 ; c1ccc(-c2ccc3oc4cccc(-c5ccc6cc(-c7nc(-c8ccc9ccccc9c8)nc(-c8cccc9c8oc8ccccc89)n7)ccc6c5)c4c3c2)cc1]

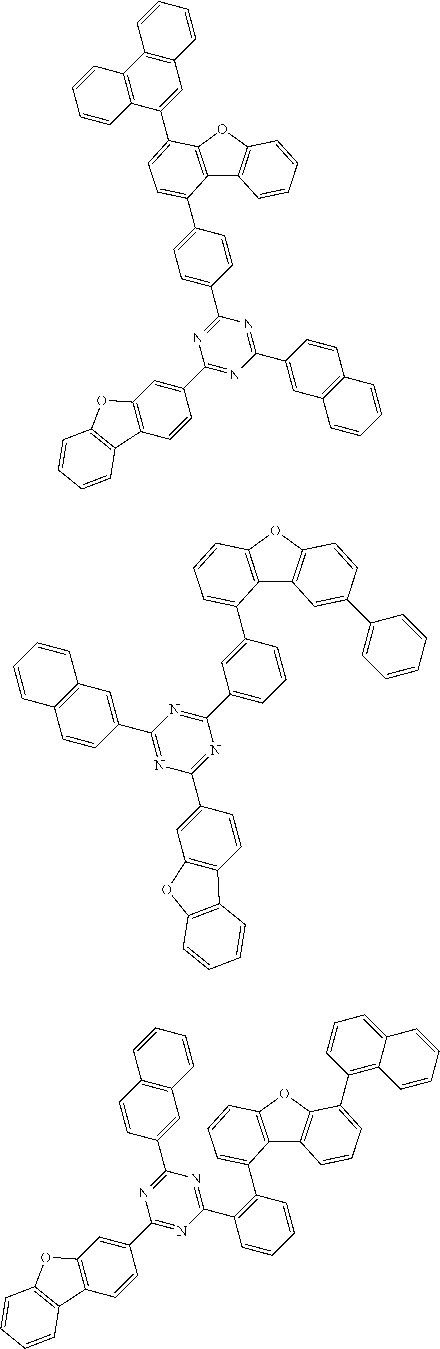 [SMILES: c1ccc2cc(-c3nc(-c4ccc(-c5ccc(-c6cc7ccccc7c7ccccc67)c6oc7ccccc7c56)cc4)nc(-c4ccc5c(c4)oc4ccccc45)n3)ccc2c1 ; c1ccc(-c2ccc3oc4cccc(-c5cccc(-c6nc(-c7ccc8ccccc8c7)nc(-c7ccc8c(c7)oc7ccccc78)n6)c5)c4c3c2)cc1 ; c1ccc(-c2cccc3oc4c(-c5cccc6ccccc56)cccc4c23)c(-c2nc(-c3ccc4ccccc4c3)nc(-c3ccc4c(c3)oc3ccccc34)n2)c1]

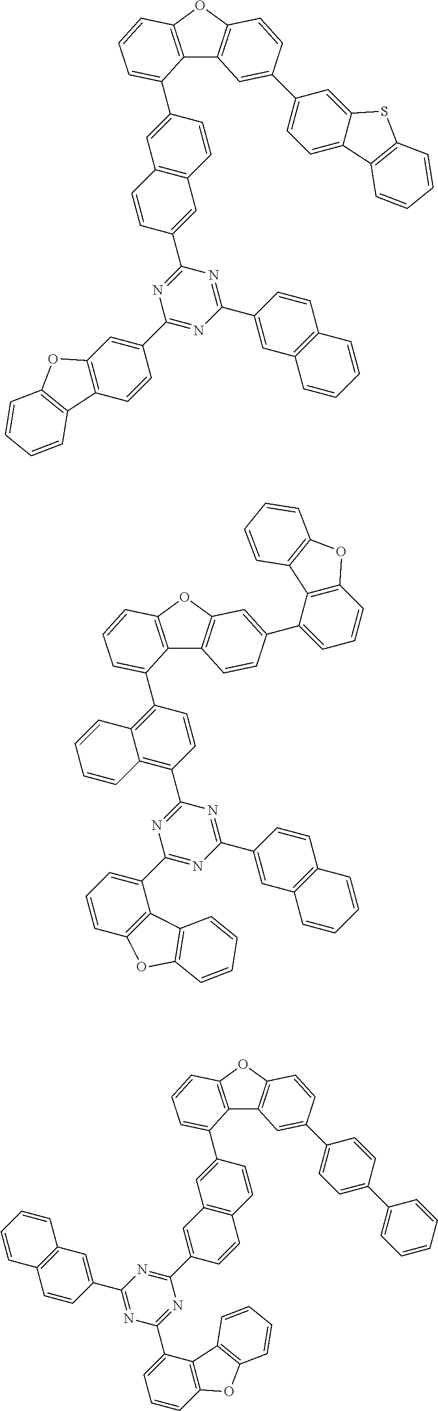 [SMILES: c1ccc2cc(-c3nc(-c4ccc5cc(-c6cccc7oc8ccc(-c9ccc%10c(c9)sc9ccccc9%10)cc8c67)ccc5c4)nc(-c4ccc5c(c4)oc4ccccc45)n3)ccc2c1 ; c1ccc2cc(-c3nc(-c4ccc(-c5cccc6oc7cc(-c8cccc9oc%10ccccc%10c89)ccc7c56)c5ccccc45)nc(-c4cccc5oc6ccccc6c45)n3)ccc2c1 ; c1ccc(-c2ccc(-c3ccc4oc5cccc(-c6ccc7ccc(-c8nc(-c9ccc%10ccccc%10c9)nc(-c9cccc%10oc%11ccccc%11c9%10)n8)cc7c6)c5c4c3)cc2)cc1]

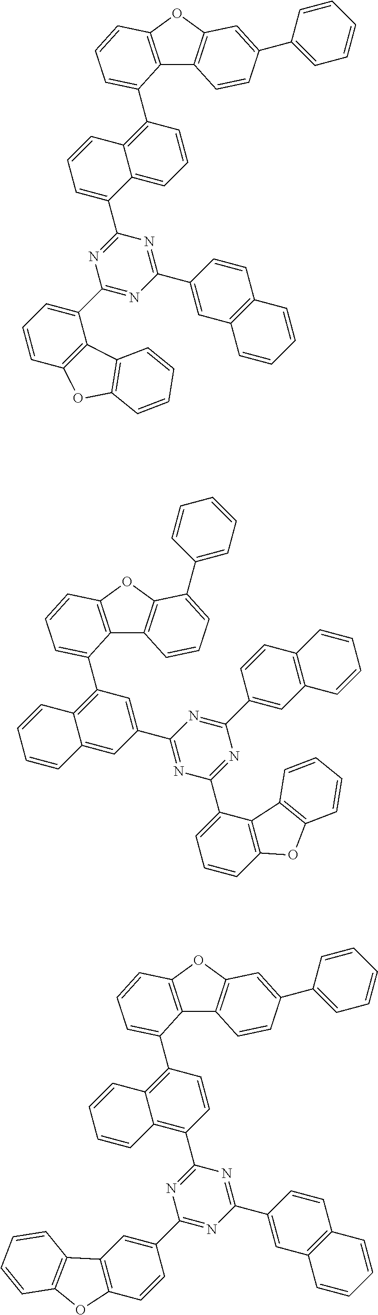 [SMILES: c1ccc(-c2ccc3c(c2)oc2cccc(-c4cccc5c(-c6nc(-c7ccc8ccccc8c7)nc(-c7cccc8oc9ccccc9c78)n6)cccc45)c23)cc1 ; c1ccc(-c2cccc3c2oc2cccc(-c4cc(-c5nc(-c6ccc7ccccc7c6)nc(-c6cccc7oc8ccccc8c67)n5)cc5ccccc45)c23)cc1 ; c1ccc(-c2ccc3c(c2)oc2cccc(-c4ccc(-c5nc(-c6ccc7ccccc7c6)nc(-c6ccc7oc8ccccc8c7c6)n5)c5ccccc45)c23)cc1]

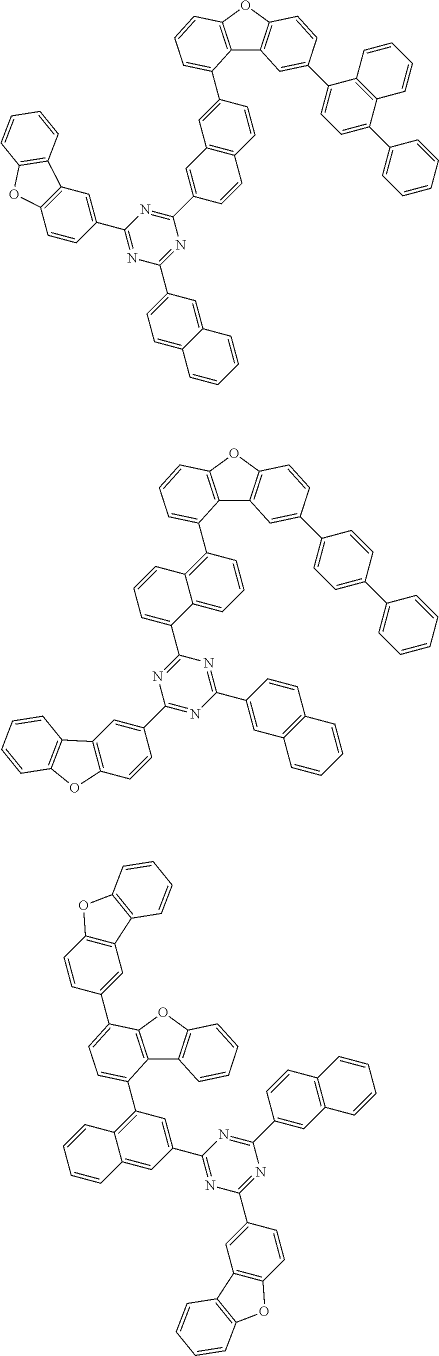 [SMILES: c1ccc(-c2ccc(-c3ccc4oc5cccc(-c6ccc7ccc(-c8nc(-c9ccc%10ccccc%10c9)nc(-c9ccc%10oc%11ccccc%11c%10c9)n8)cc7c6)c5c4c3)c3ccccc23)cc1 ; c1ccc(-c2ccc(-c3ccc4oc5cccc(-c6cccc7c(-c8nc(-c9ccc%10ccccc%10c9)nc(-c9ccc%10oc%11ccccc%11c%10c9)n8)cccc67)c5c4c3)cc2)cc1 ; c1ccc2cc(-c3nc(-c4cc(-c5ccc(-c6ccc7oc8ccccc8c7c6)c6oc7ccccc7c56)c5ccccc5c4)nc(-c4ccc5oc6ccccc6c5c4)n3)ccc2c1]

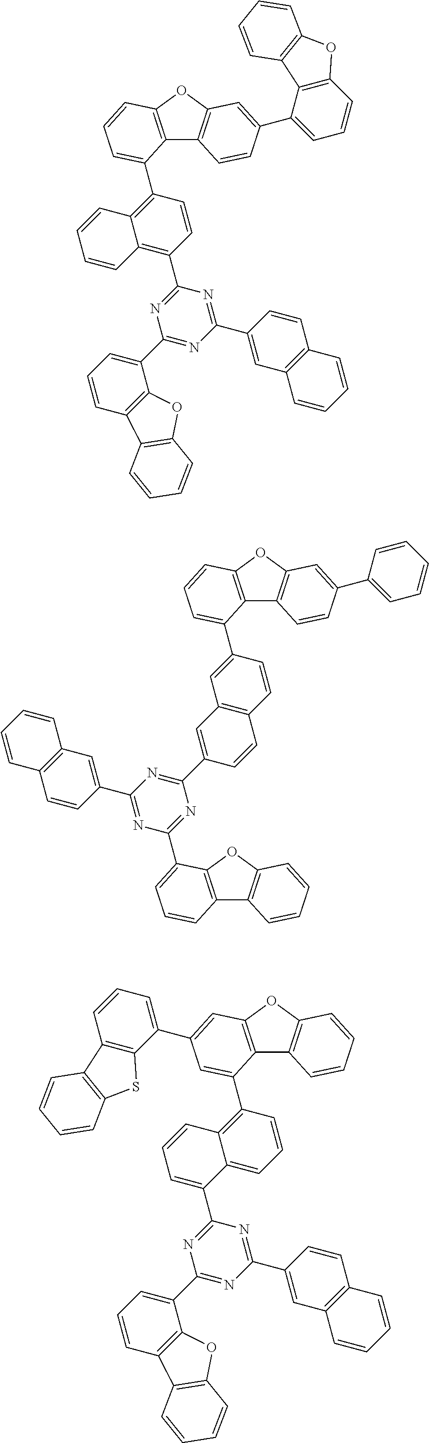 [SMILES: c1ccc2cc(-c3nc(-c4ccc(-c5cccc6oc7cc(-c8cccc9oc%10ccccc%10c89)ccc7c56)c5ccccc45)nc(-c4cccc5c4oc4ccccc45)n3)ccc2c1 ; c1ccc(-c2ccc3c(c2)oc2cccc(-c4ccc5ccc(-c6nc(-c7ccc8ccccc8c7)nc(-c7cccc8c7oc7ccccc78)n6)cc5c4)c23)cc1 ; c1ccc2cc(-c3nc(-c4cccc5c(-c6cc(-c7cccc8c7sc7ccccc78)cc7oc8ccccc8c67)cccc45)nc(-c4cccc5c4oc4ccccc45)n3)ccc2c1]

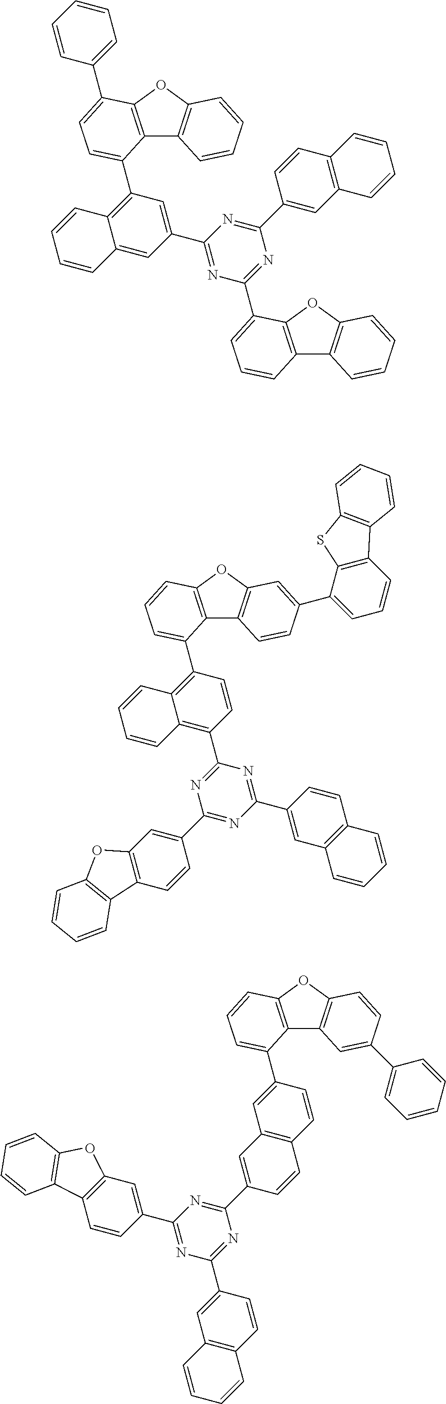 [SMILES: c1ccc(-c2ccc(-c3cc(-c4nc(-c5ccc6ccccc6c5)nc(-c5cccc6c5oc5ccccc56)n4)cc4ccccc34)c3c2oc2ccccc23)cc1 ; c1ccc2cc(-c3nc(-c4ccc5c(c4)oc4ccccc45)nc(-c4ccc(-c5cccc6oc7cc(-c8cccc9c8sc8ccccc89)ccc7c56)c5ccccc45)n3)ccc2c1 ; c1ccc(-c2ccc3oc4cccc(-c5ccc6ccc(-c7nc(-c8ccc9ccccc9c8)nc(-c8ccc9c(c8)oc8ccccc89)n7)cc6c5)c4c3c2)cc1]

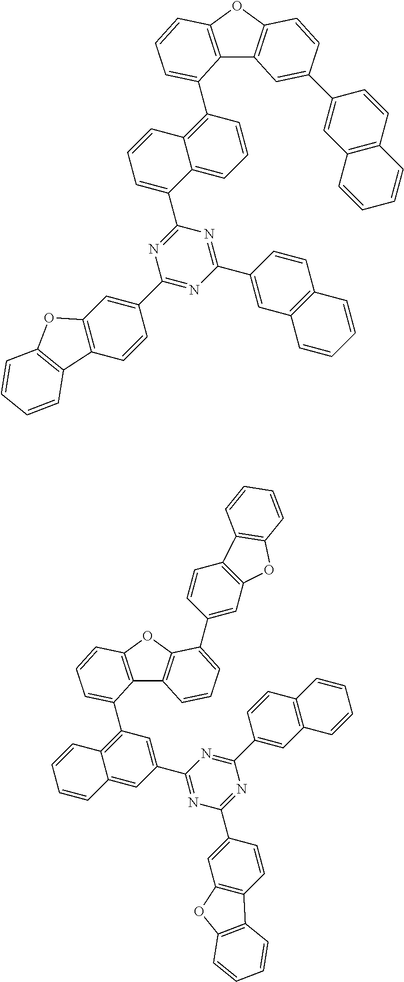 [SMILES: c1ccc2cc(-c3ccc4oc5cccc(-c6cccc7c(-c8nc(-c9ccc%10ccccc%10c9)nc(-c9ccc%10c(c9)oc9ccccc9%10)n8)cccc67)c5c4c3)ccc2c1 ; c1ccc2cc(-c3nc(-c4cc(-c5cccc6oc7c(-c8ccc9c(c8)oc8ccccc89)cccc7c56)c5ccccc5c4)nc(-c4ccc5c(c4)oc4ccccc45)n3)ccc2c1]

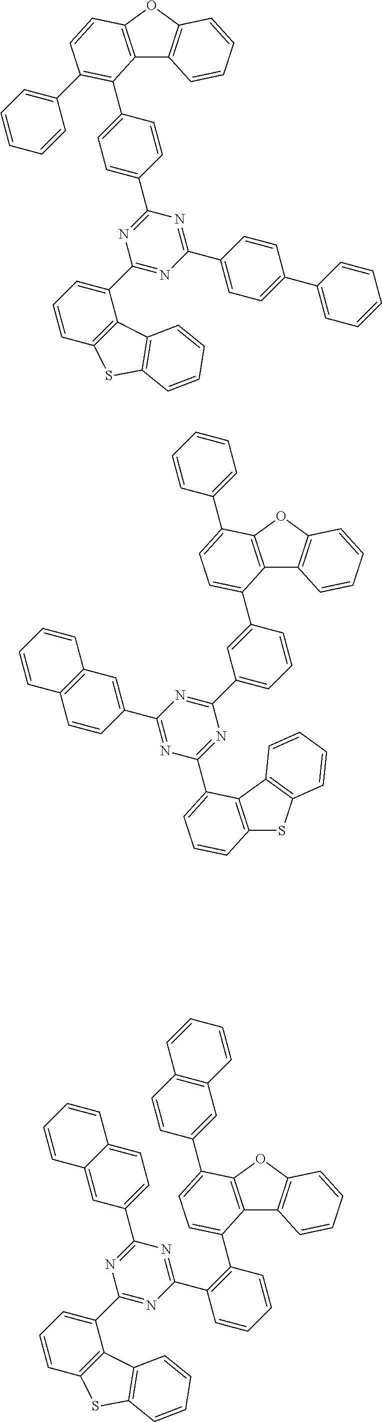 [SMILES: c1ccc(-c2ccc(-c3nc(-c4ccc(-c5c(-c6ccccc6)ccc6oc7ccccc7c56)cc4)nc(-c4cccc5sc6ccccc6c45)n3)cc2)cc1 ; c1ccc(-c2ccc(-c3cccc(-c4nc(-c5ccc6ccccc6c5)nc(-c5cccc6sc7ccccc7c56)n4)c3)c3c2oc2ccccc23)cc1 ; c1ccc(-c2ccc(-c3ccc4ccccc4c3)c3oc4ccccc4c23)c(-c2nc(-c3ccc4ccccc4c3)nc(-c3cccc4sc5ccccc5c34)n2)c1]

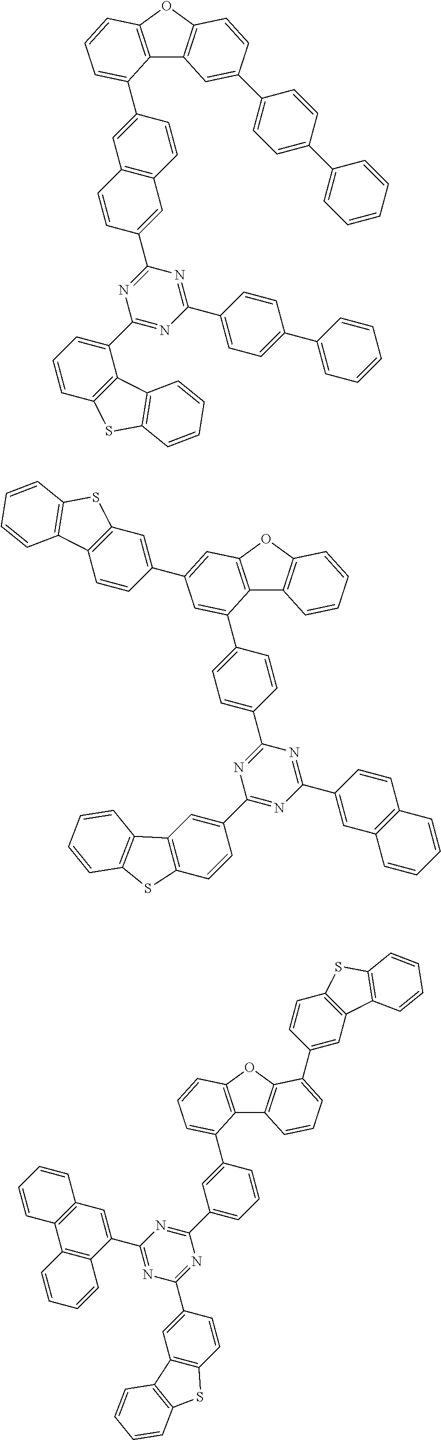 [SMILES: c1ccc(-c2ccc(-c3ccc4oc5cccc(-c6ccc7cc(-c8nc(-c9ccc(-c%10ccccc%10)cc9)nc(-c9cccc%10sc%11ccccc%11c9%10)n8)ccc7c6)c5c4c3)cc2)cc1 ; c1ccc2cc(-c3nc(-c4ccc(-c5cc(-c6ccc7c(c6)sc6ccccc67)cc6oc7ccccc7c56)cc4)nc(-c4ccc5sc6ccccc6c5c4)n3)ccc2c1 ; c1cc(-c2nc(-c3ccc4sc5ccccc5c4c3)nc(-c3cc4ccccc4c4ccccc34)n2)cc(-c2cccc3oc4c(-c5ccc6sc7ccccc7c6c5)cccc4c23)c1]

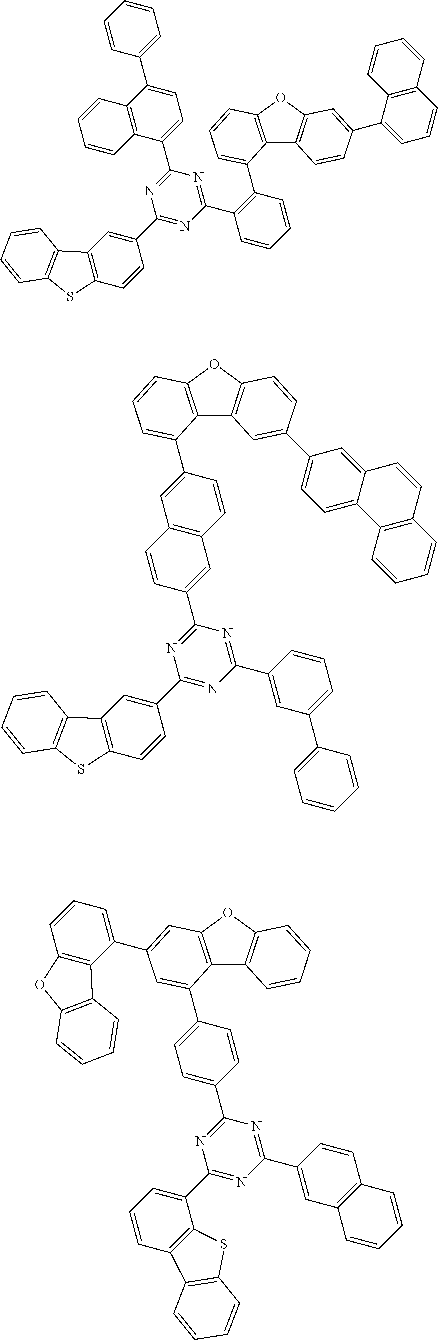 [SMILES: c1ccc(-c2ccc(-c3nc(-c4ccc5sc6ccccc6c5c4)nc(-c4ccccc4-c4cccc5oc6cc(-c7cccc8ccccc78)ccc6c45)n3)c3ccccc23)cc1 ; c1ccc(-c2cccc(-c3nc(-c4ccc5cc(-c6cccc7oc8ccc(-c9ccc%10c(ccc%11ccccc%11%10)c9)cc8c67)ccc5c4)nc(-c4ccc5sc6ccccc6c5c4)n3)c2)cc1 ; c1ccc2cc(-c3nc(-c4ccc(-c5cc(-c6cccc7oc8ccccc8c67)cc6oc7ccccc7c56)cc4)nc(-c4cccc5c4sc4ccccc45)n3)ccc2c1]

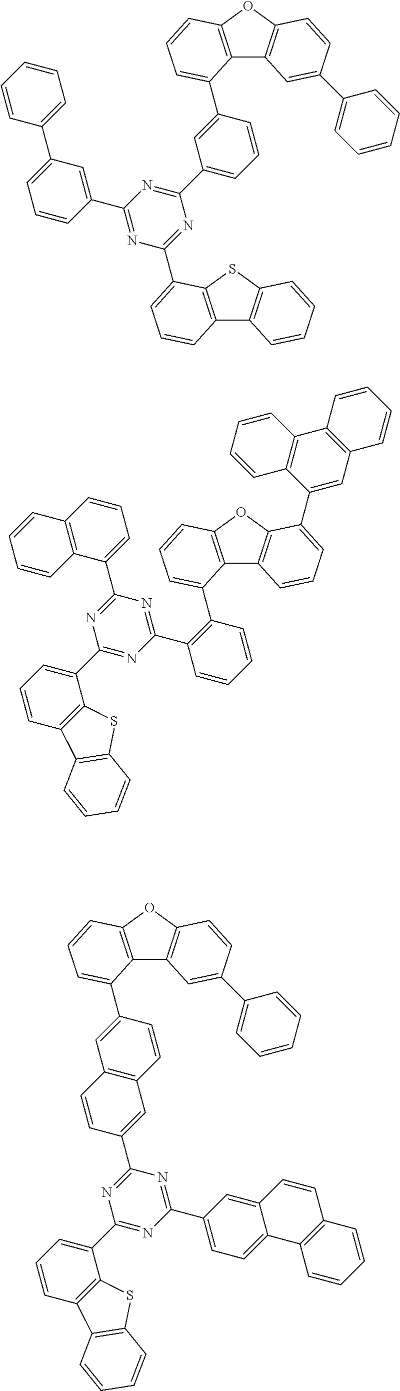 [SMILES: c1ccc(-c2cccc(-c3nc(-c4cccc(-c5cccc6oc7ccc(-c8ccccc8)cc7c56)c4)nc(-c4cccc5c4sc4ccccc45)n3)c2)cc1 ; c1ccc(-c2cccc3oc4c(-c5cc6ccccc6c6ccccc56)cccc4c23)c(-c2nc(-c3cccc4ccccc34)nc(-c3cccc4c3sc3ccccc34)n2)c1 ; c1ccc(-c2ccc3oc4cccc(-c5ccc6cc(-c7nc(-c8ccc9c(ccc%10ccccc%109)c8)nc(-c8cccc9c8sc8ccccc89)n7)ccc6c5)c4c3c2)cc1]

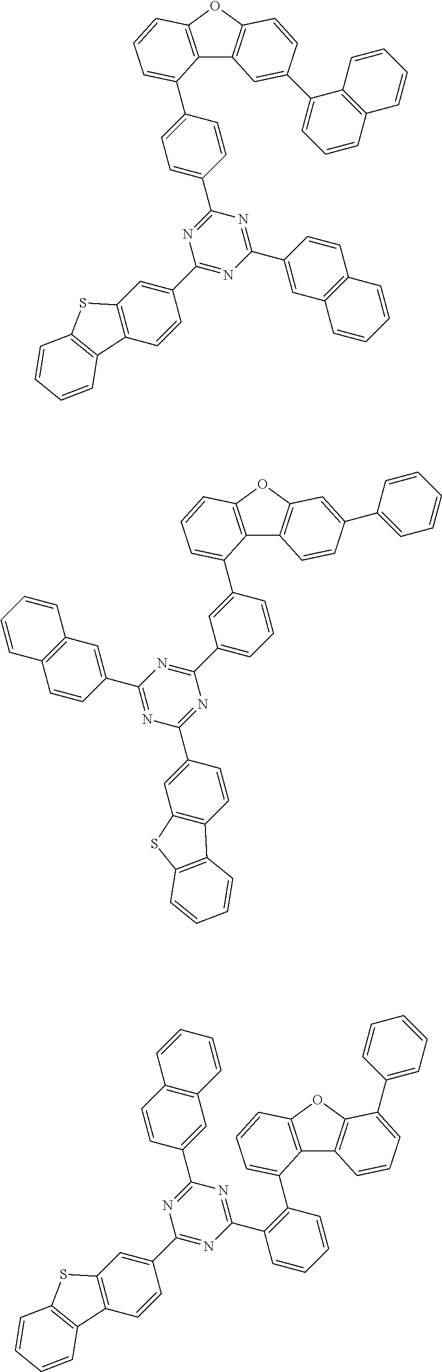 [SMILES: c1ccc2cc(-c3nc(-c4ccc(-c5cccc6oc7ccc(-c8cccc9ccccc89)cc7c56)cc4)nc(-c4ccc5c(c4)sc4ccccc45)n3)ccc2c1 ; c1ccc(-c2ccc3c(c2)oc2cccc(-c4cccc(-c5nc(-c6ccc7ccccc7c6)nc(-c6ccc7c(c6)sc6ccccc67)n5)c4)c23)cc1 ; c1ccc(-c2cccc3c2oc2cccc(-c4ccccc4-c4nc(-c5ccc6ccccc6c5)nc(-c5ccc6c(c5)sc5ccccc56)n4)c23)cc1]

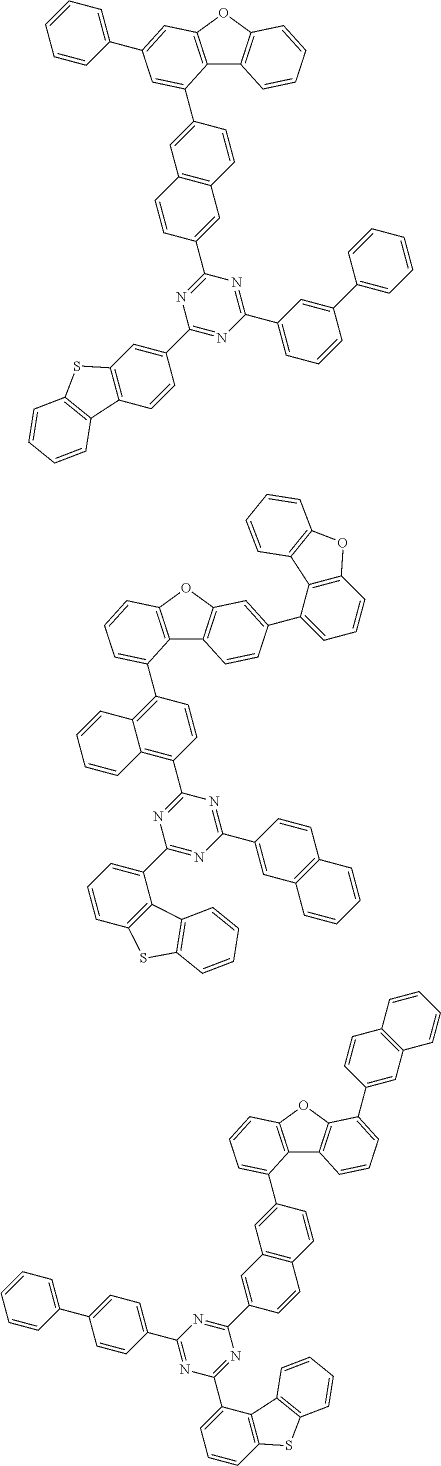 [SMILES: c1ccc(-c2cccc(-c3nc(-c4ccc5cc(-c6cc(-c7ccccc7)cc7oc8ccccc8c67)ccc5c4)nc(-c4ccc5c(c4)sc4ccccc45)n3)c2)cc1 ; c1ccc2cc(-c3nc(-c4ccc(-c5cccc6oc7cc(-c8cccc9oc%10ccccc%10c89)ccc7c56)c5ccccc45)nc(-c4cccc5sc6ccccc6c45)n3)ccc2c1 ; c1ccc(-c2ccc(-c3nc(-c4ccc5ccc(-c6cccc7oc8c(-c9ccc%10ccccc%10c9)cccc8c67)cc5c4)nc(-c4cccc5sc6ccccc6c45)n3)cc2)cc1]

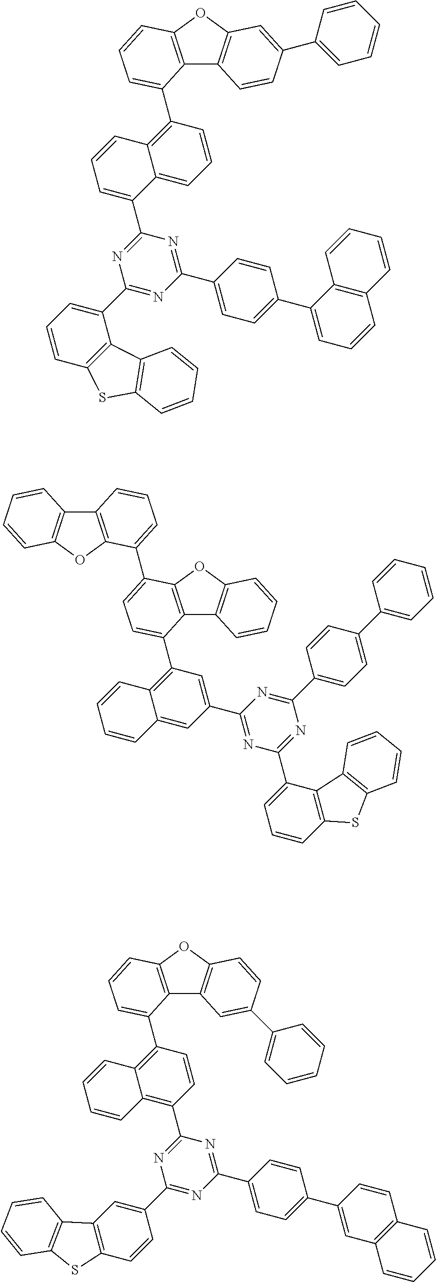 [SMILES: c1ccc(-c2ccc3c(c2)oc2cccc(-c4cccc5c(-c6nc(-c7ccc(-c8cccc9ccccc89)cc7)nc(-c7cccc8sc9ccccc9c78)n6)cccc45)c23)cc1 ; c1ccc(-c2ccc(-c3nc(-c4cc(-c5ccc(-c6cccc7c6oc6ccccc67)c6oc7ccccc7c56)c5ccccc5c4)nc(-c4cccc5sc6ccccc6c45)n3)cc2)cc1 ; c1ccc(-c2ccc3oc4cccc(-c5ccc(-c6nc(-c7ccc(-c8ccc9ccccc9c8)cc7)nc(-c7ccc8sc9ccccc9c8c7)n6)c6ccccc56)c4c3c2)cc1]

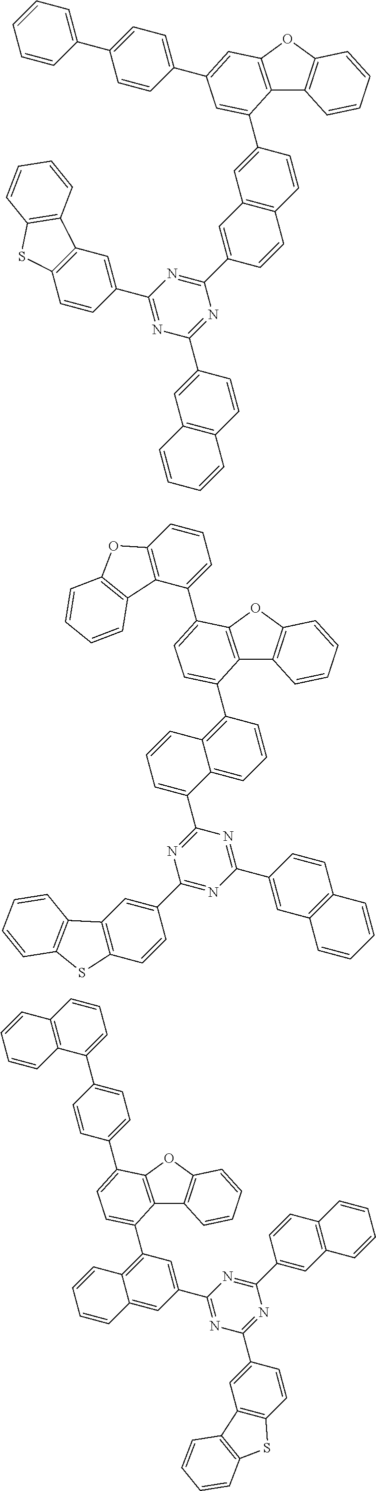

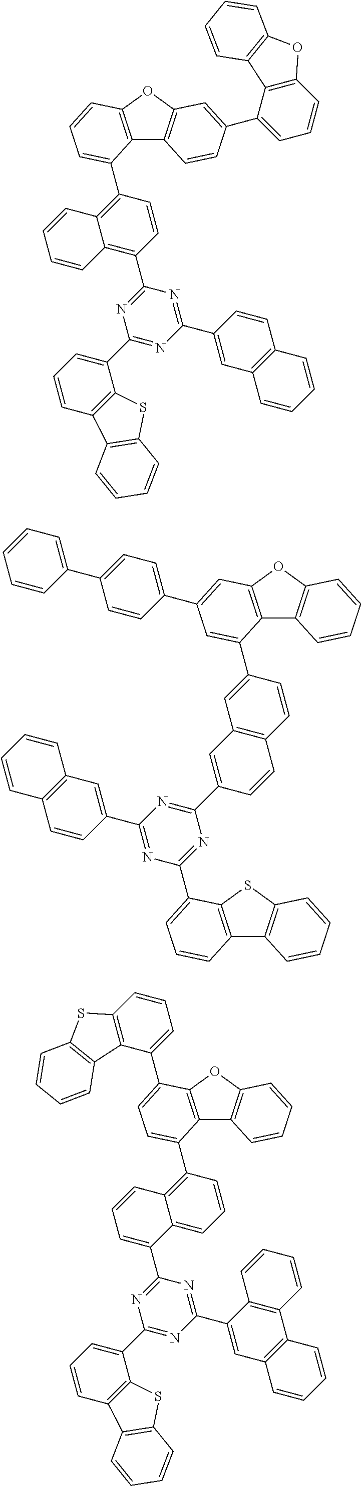 [SMILES: c1ccc2cc(-c3nc(-c4ccc(-c5cccc6oc7cc(-c8cccc9oc%10ccccc%10c89)ccc7c56)c5ccccc45)nc(-c4cccc5c4sc4ccccc45)n3)ccc2c1 ; c1ccc(-c2ccc(-c3cc(-c4ccc5ccc(-c6nc(-c7ccc8ccccc8c7)nc(-c7cccc8c7sc7ccccc78)n6)cc5c4)c4c(c3)oc3ccccc34)cc2)cc1 ; c1ccc2c(c1)cc(-c1nc(-c3cccc4c(-c5ccc(-c6cccc7sc8ccccc8c67)c6oc7ccccc7c56)cccc34)nc(-c3cccc4c3sc3ccccc34)n1)c1ccccc12]

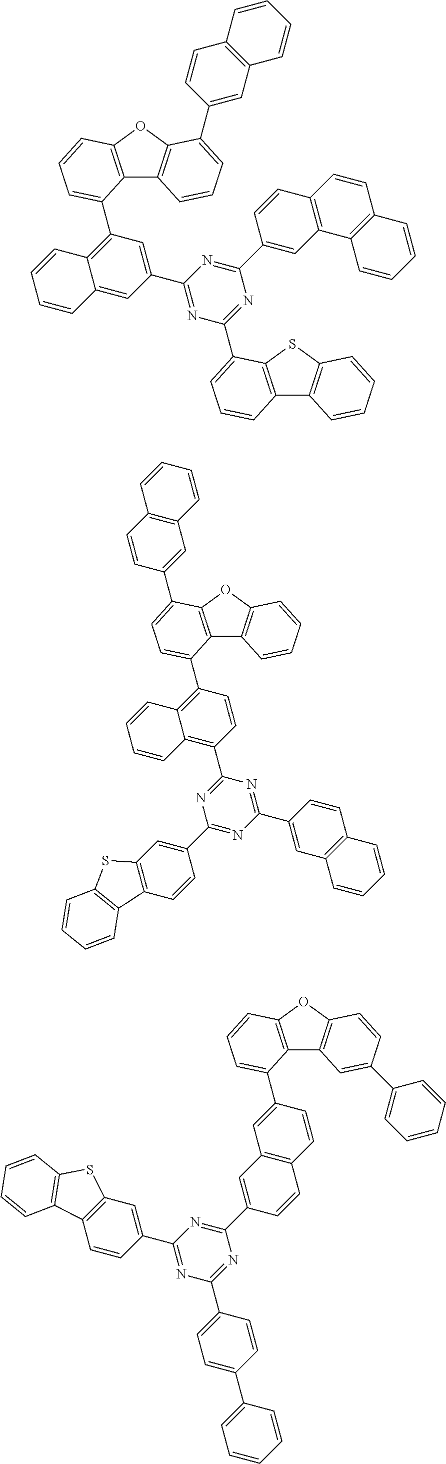 [SMILES: c1ccc2cc(-c3cccc4c3oc3cccc(-c5cc(-c6nc(-c7ccc8ccc9ccccc9c8c7)nc(-c7cccc8c7sc7ccccc78)n6)cc6ccccc56)c34)ccc2c1 ; c1ccc2cc(-c3nc(-c4ccc5c(c4)sc4ccccc45)nc(-c4ccc(-c5ccc(-c6ccc7ccccc7c6)c6oc7ccccc7c56)c5ccccc45)n3)ccc2c1 ; c1ccc(-c2ccc(-c3nc(-c4ccc5ccc(-c6cccc7oc8ccc(-c9ccccc9)cc8c67)cc5c4)nc(-c4ccc5c(c4)sc4ccccc45)n3)cc2)cc1]

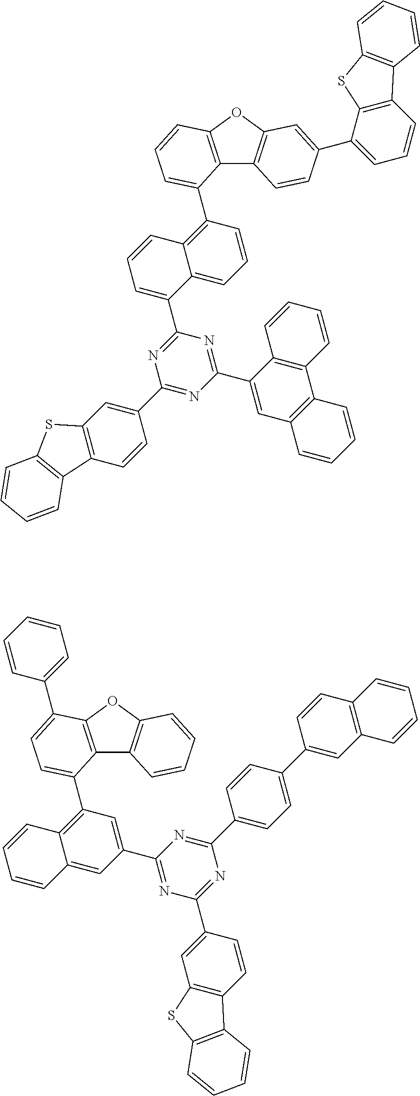 [SMILES: c1ccc2c(c1)cc(-c1nc(-c3ccc4c(c3)sc3ccccc34)nc(-c3cccc4c(-c5cccc6oc7cc(-c8cccc9c8sc8ccccc89)ccc7c56)cccc34)n1)c1ccccc12 ; c1ccc(-c2ccc(-c3cc(-c4nc(-c5ccc(-c6ccc7ccccc7c6)cc5)nc(-c5ccc6c(c5)sc5ccccc56)n4)cc4ccccc34)c3c2oc2ccccc23)cc1]

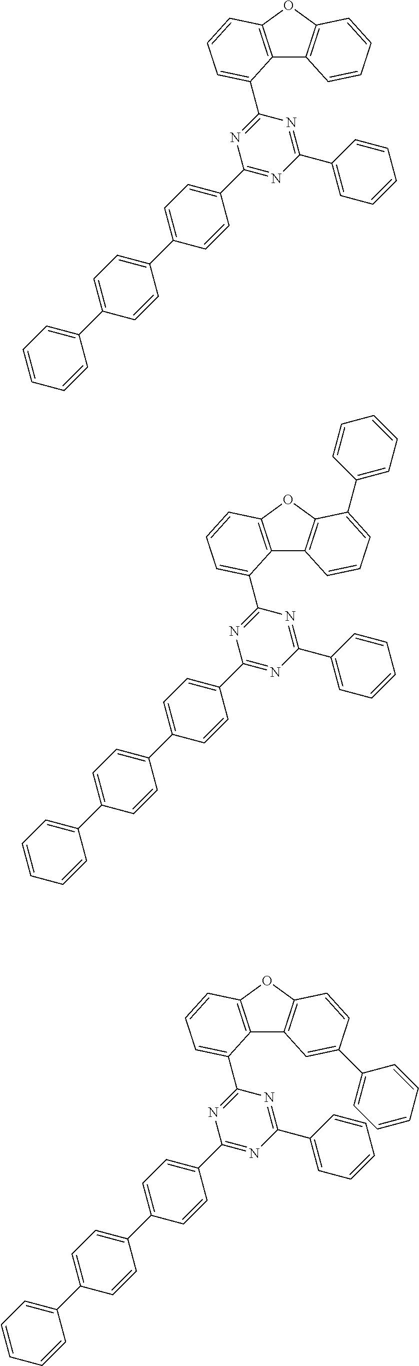 [SMILES: c1ccc(-c2ccc(-c3ccc(-c4nc(-c5ccccc5)nc(-c5cccc6oc7ccccc7c56)n4)cc3)cc2)cc1 ; c1ccc(-c2ccc(-c3ccc(-c4nc(-c5ccccc5)nc(-c5cccc6oc7c(-c8ccccc8)cccc7c56)n4)cc3)cc2)cc1 ; c1ccc(-c2ccc(-c3ccc(-c4nc(-c5ccccc5)nc(-c5cccc6oc7ccc(-c8ccccc8)cc7c56)n4)cc3)cc2)cc1]

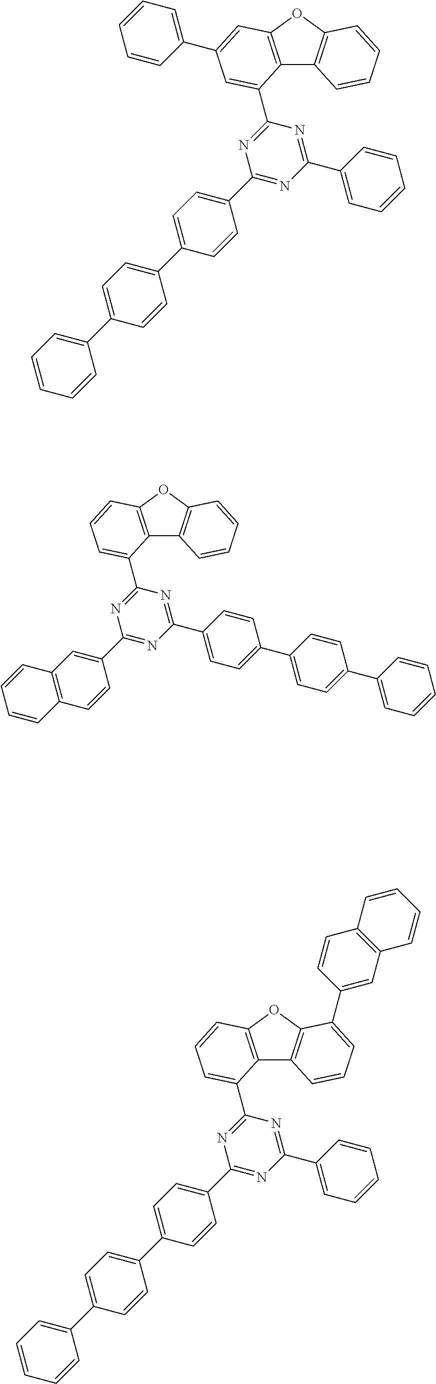 [SMILES: c1ccc(-c2ccc(-c3ccc(-c4nc(-c5ccccc5)nc(-c5cc(-c6ccccc6)cc6oc7ccccc7c56)n4)cc3)cc2)cc1 ; c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3ccc(-c4nc(-c5ccccc5)nc(-c5cccc6oc7c(-c8ccc9ccccc9c8)cccc7c56)n4)cc3)cc2)cc1]

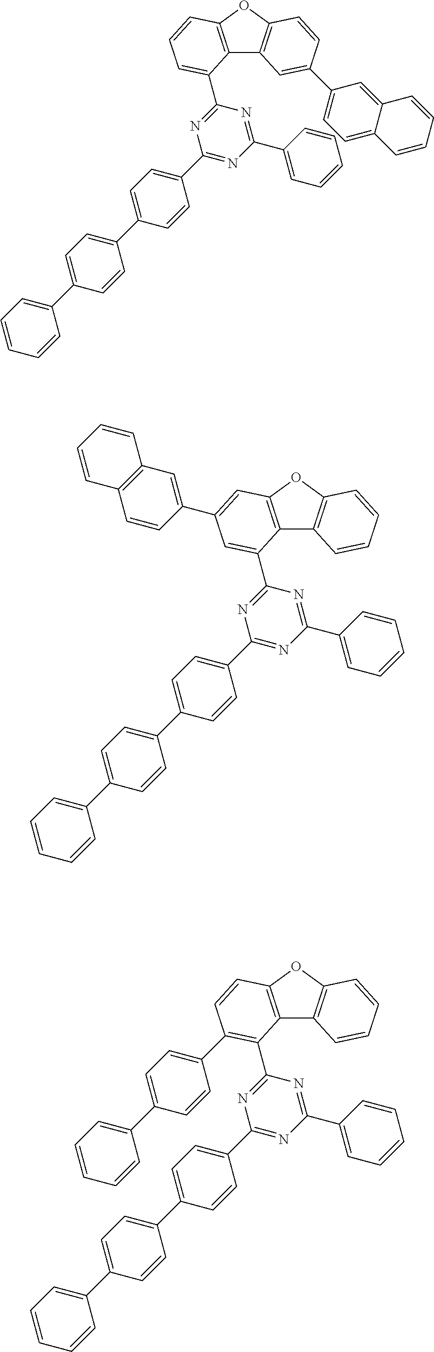 [SMILES: c1ccc(-c2ccc(-c3ccc(-c4nc(-c5ccccc5)nc(-c5cccc6oc7ccc(-c8ccc9ccccc9c8)cc7c56)n4)cc3)cc2)cc1 ; c1ccc(-c2ccc(-c3ccc(-c4nc(-c5ccccc5)nc(-c5cc(-c6ccc7ccccc7c6)cc6oc7ccccc7c56)n4)cc3)cc2)cc1 ; c1ccc(-c2ccc(-c3ccc(-c4nc(-c5ccccc5)nc(-c5c(-c6ccc(-c7ccccc7)cc6)ccc6oc7ccccc7c56)n4)cc3)cc2)cc1]

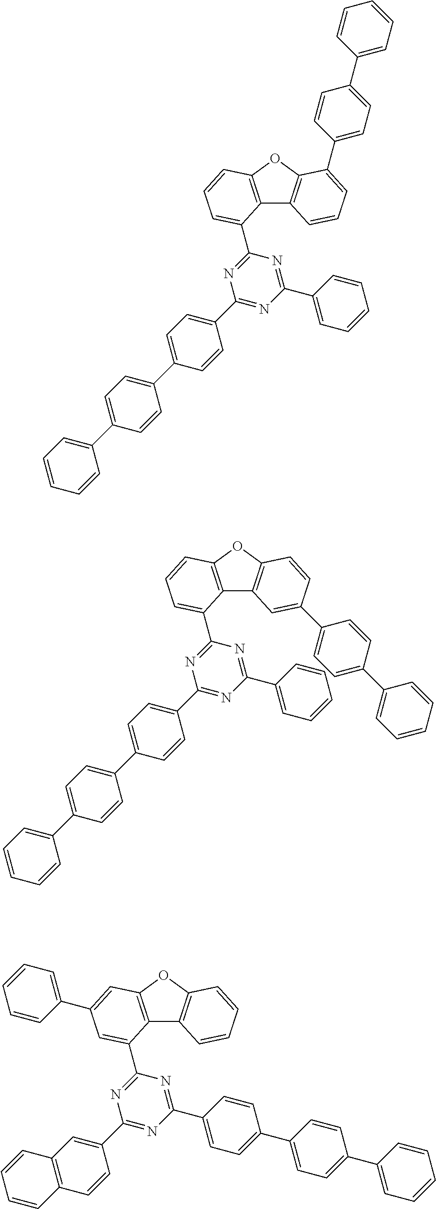 [SMILES: c1ccc(-c2ccc(-c3ccc(-c4nc(-c5ccccc5)nc(-c5cccc6oc7c(-c8ccc(-c9ccccc9)cc8)cccc7c56)n4)cc3)cc2)cc1 ; c1ccc(-c2ccc(-c3ccc(-c4nc(-c5ccccc5)nc(-c5cccc6oc7ccc(-c8ccc(-c9ccccc9)cc8)cc7c56)n4)cc3)cc2)cc1 ; c1ccc(-c2ccc(-c3ccc(-c4nc(-c5ccc6ccccc6c5)nc(-c5cc(-c6ccccc6)cc6oc7ccccc7c56)n4)cc3)cc2)cc1]

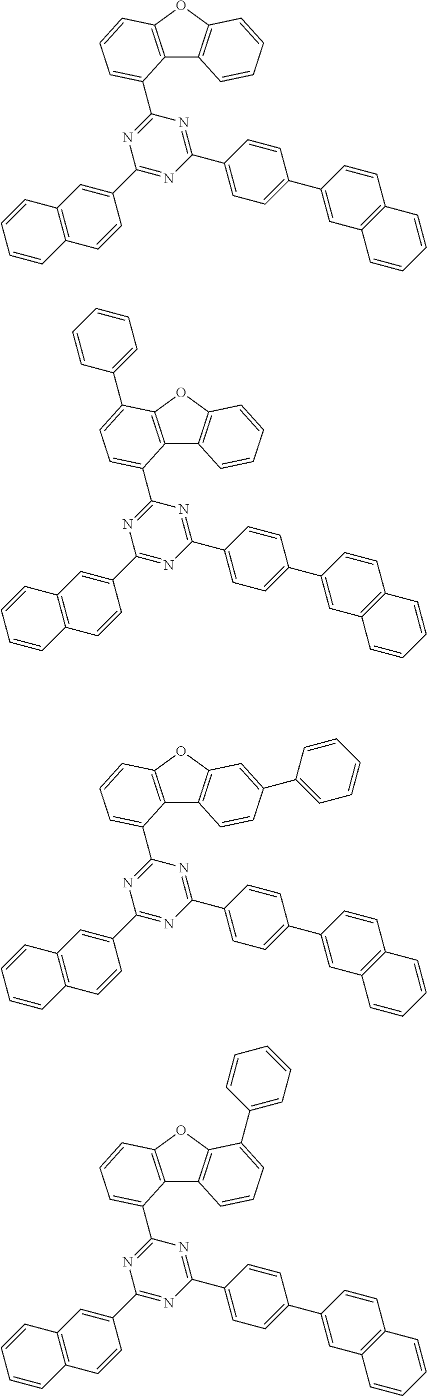 [SMILES: c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3nc(-c4ccc(-c5ccc6ccccc6c5)cc4)nc(-c4ccc5ccccc5c4)n3)c3c2oc2ccccc23)cc1 ; c1ccc(-c2ccc3c(c2)oc2cccc(-c4nc(-c5ccc(-c6ccc7ccccc7c6)cc5)nc(-c5ccc6ccccc6c5)n4)c23)cc1 ; c1ccc(-c2cccc3c2oc2cccc(-c4nc(-c5ccc(-c6ccc7ccccc7c6)cc5)nc(-c5ccc6ccccc6c5)n4)c23)cc1]

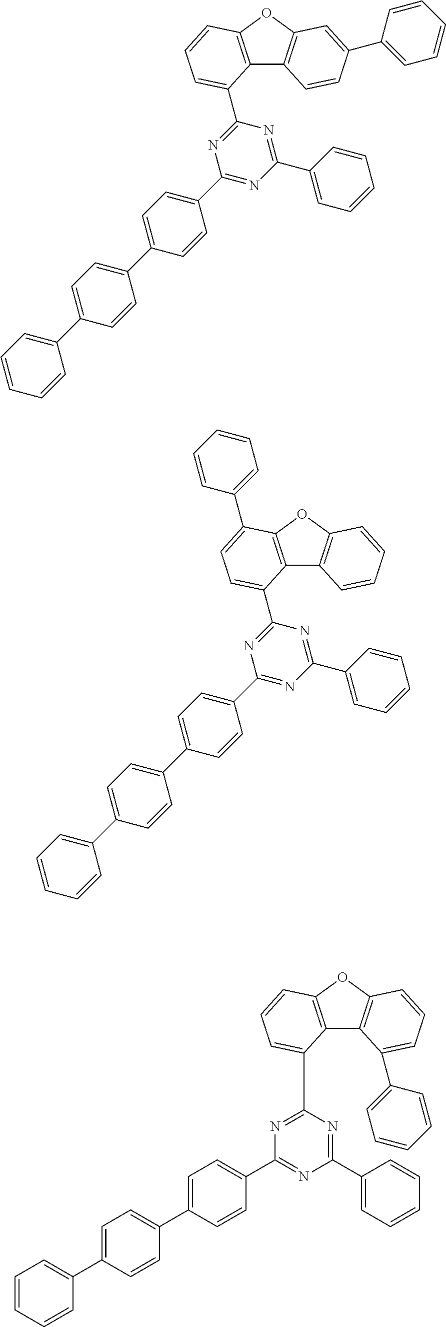 [SMILES: c1ccc(-c2ccc(-c3ccc(-c4nc(-c5ccccc5)nc(-c5cccc6oc7cc(-c8ccccc8)ccc7c56)n4)cc3)cc2)cc1 ; c1ccc(-c2ccc(-c3ccc(-c4nc(-c5ccccc5)nc(-c5ccc(-c6ccccc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccccc8)c7c56)n4)cc3)cc2)cc1]

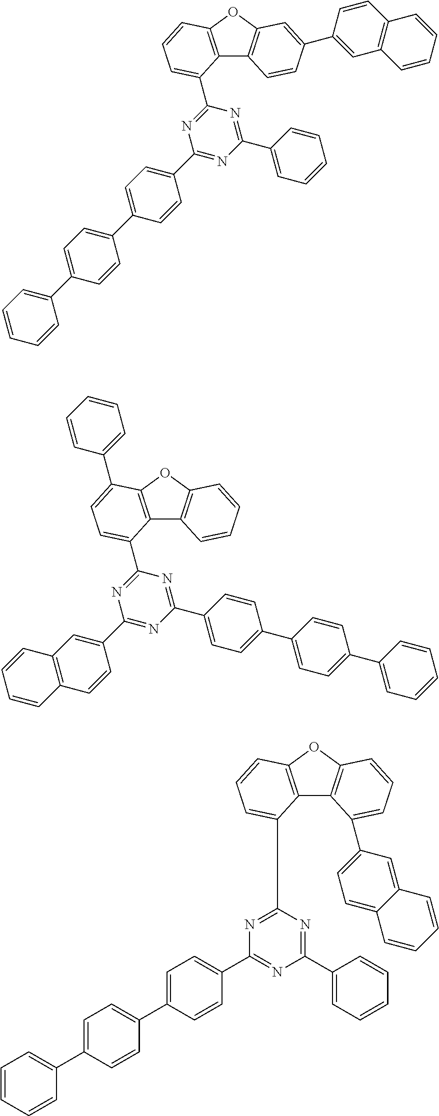 [SMILES: c1ccc(-c2ccc(-c3ccc(-c4nc(-c5ccccc5)nc(-c5cccc6oc7cc(-c8ccc9ccccc9c8)ccc7c56)n4)cc3)cc2)cc1 ; c1ccc(-c2ccc(-c3ccc(-c4nc(-c5ccc6ccccc6c5)nc(-c5ccc(-c6ccccc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9ccccc9c8)c7c56)n4)cc3)cc2)cc1]

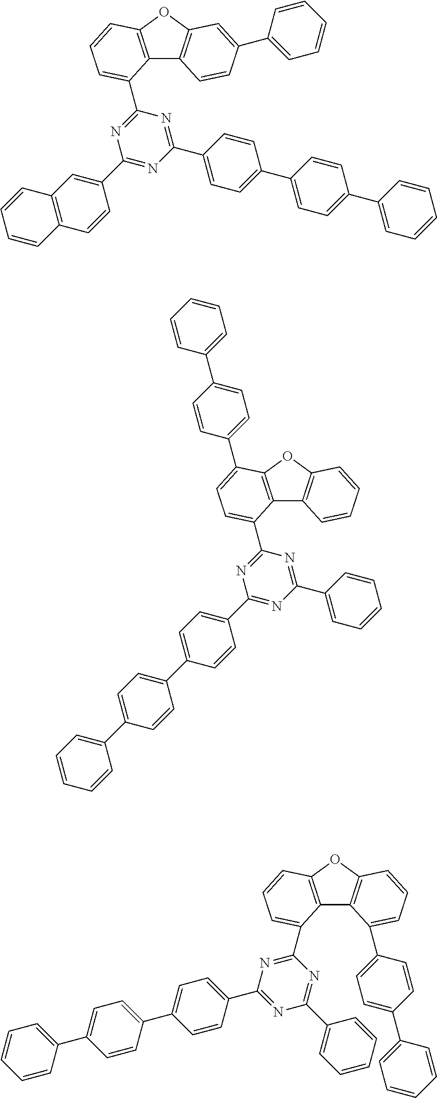 [SMILES: c1ccc(-c2ccc(-c3ccc(-c4nc(-c5ccc6ccccc6c5)nc(-c5cccc6oc7cc(-c8ccccc8)ccc7c56)n4)cc3)cc2)cc1 ; c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1]

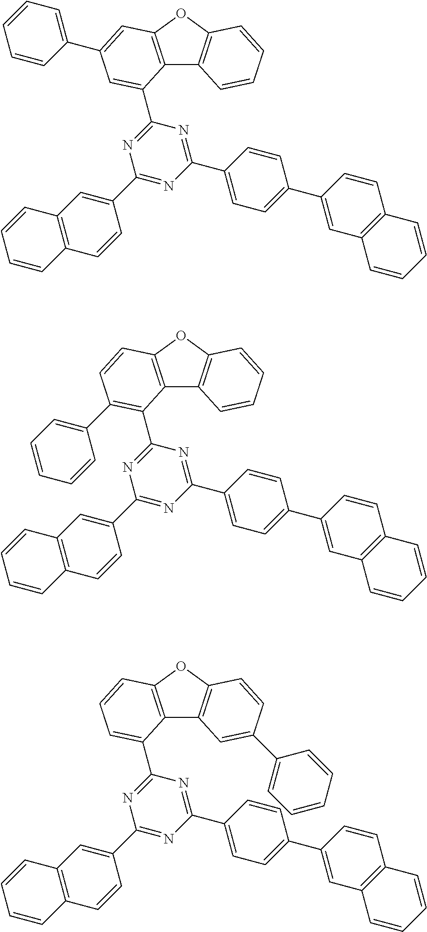 [SMILES: c1ccc(-c2cc(-c3nc(-c4ccc(-c5ccc6ccccc6c5)cc4)nc(-c4ccc5ccccc5c4)n3)c3c(c2)oc2ccccc23)cc1 ; c1ccc(-c2ccc3oc4ccccc4c3c2-c2nc(-c3ccc(-c4ccc5ccccc5c4)cc3)nc(-c3ccc4ccccc4c3)n2)cc1 ; c1ccc(-c2ccc3oc4cccc(-c5nc(-c6ccc(-c7ccc8ccccc8c7)cc6)nc(-c6ccc7ccccc7c6)n5)c4c3c2)cc1]

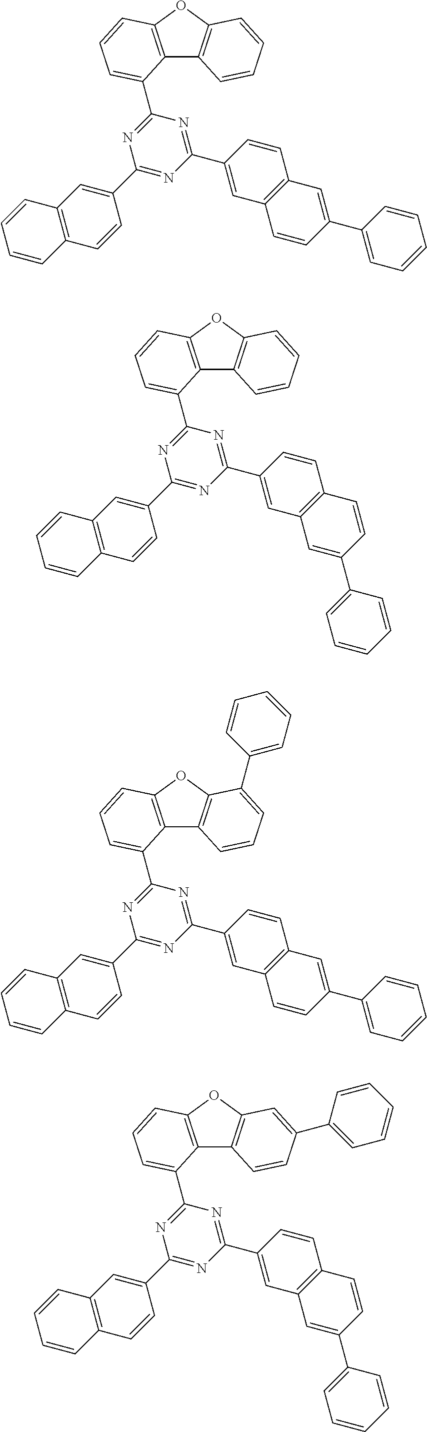 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc3ccc(-c4nc(-c5ccc6ccccc6c5)nc(-c5cccc6oc7ccccc7c56)n4)cc3c2)cc1 ; c1ccc(-c2ccc3cc(-c4nc(-c5ccc6ccccc6c5)nc(-c5cccc6oc7c(-c8ccccc8)cccc7c56)n4)ccc3c2)cc1 ; c1ccc(-c2ccc3ccc(-c4nc(-c5ccc6ccccc6c5)nc(-c5cccc6oc7cc(-c8ccccc8)ccc7c56)n4)cc3c2)cc1]

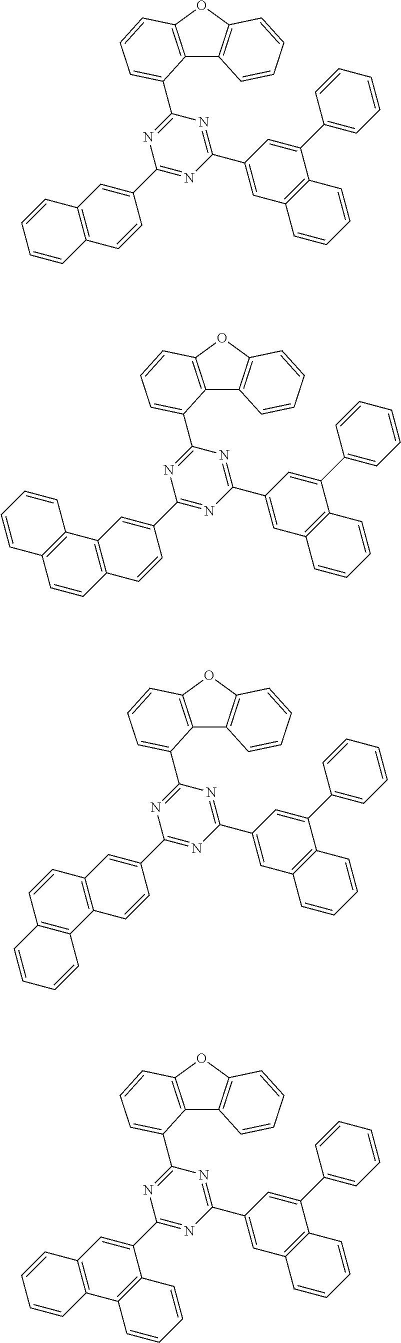 [SMILES: c1ccc(-c2cc(-c3nc(-c4ccc5ccccc5c4)nc(-c4cccc5oc6ccccc6c45)n3)cc3ccccc23)cc1 ; c1ccc(-c2cc(-c3nc(-c4ccc5ccc6ccccc6c5c4)nc(-c4cccc5oc6ccccc6c45)n3)cc3ccccc23)cc1 ; c1ccc(-c2cc(-c3nc(-c4ccc5c(ccc6ccccc65)c4)nc(-c4cccc5oc6ccccc6c45)n3)cc3ccccc23)cc1 ; c1ccc(-c2cc(-c3nc(-c4cc5ccccc5c5ccccc45)nc(-c4cccc5oc6ccccc6c45)n3)cc3ccccc23)cc1]

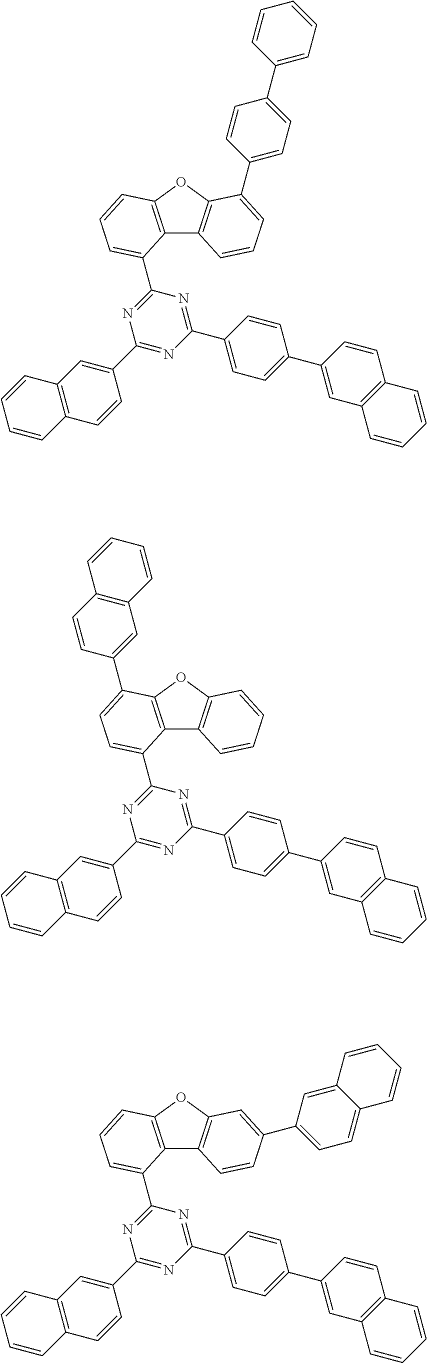 [SMILES: c1ccc(-c2ccc(-c3cccc4c3oc3cccc(-c5nc(-c6ccc(-c7ccc8ccccc8c7)cc6)nc(-c6ccc7ccccc7c6)n5)c34)cc2)cc1 ; c1ccc2cc(-c3ccc(-c4nc(-c5ccc6ccccc6c5)nc(-c5ccc(-c6ccc7ccccc7c6)c6oc7ccccc7c56)n4)cc3)ccc2c1 ; c1ccc2cc(-c3ccc(-c4nc(-c5ccc6ccccc6c5)nc(-c5cccc6oc7cc(-c8ccc9ccccc9c8)ccc7c56)n4)cc3)ccc2c1]

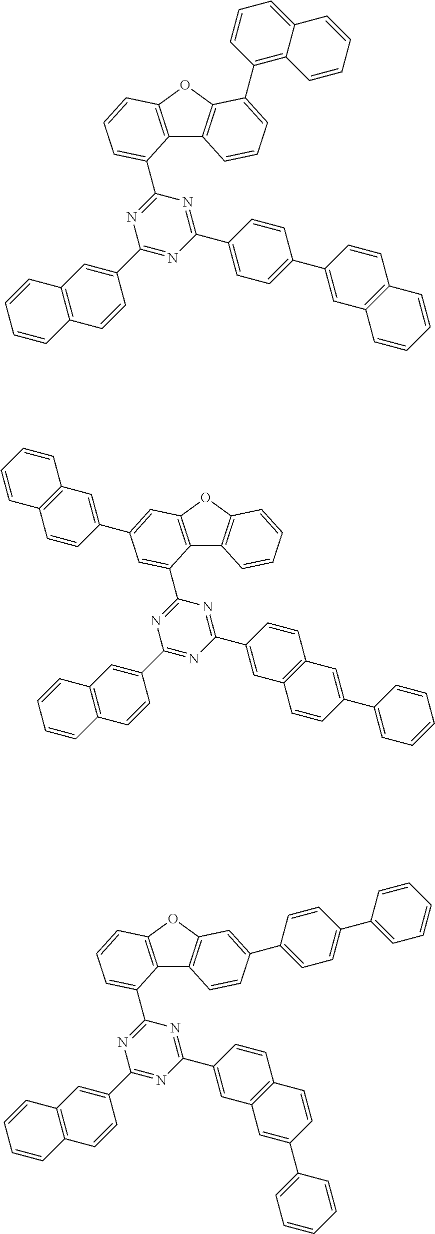 [SMILES: c1ccc2cc(-c3ccc(-c4nc(-c5ccc6ccccc6c5)nc(-c5cccc6oc7c(-c8cccc9ccccc89)cccc7c56)n4)cc3)ccc2c1 ; c1ccc(-c2ccc3cc(-c4nc(-c5ccc6ccccc6c5)nc(-c5cc(-c6ccc7ccccc7c6)cc6oc7ccccc7c56)n4)ccc3c2)cc1 ; c1ccc(-c2ccc(-c3ccc4c(c3)oc3cccc(-c5nc(-c6ccc7ccccc7c6)nc(-c6ccc7ccc(-c8ccccc8)cc7c6)n5)c34)cc2)cc1]

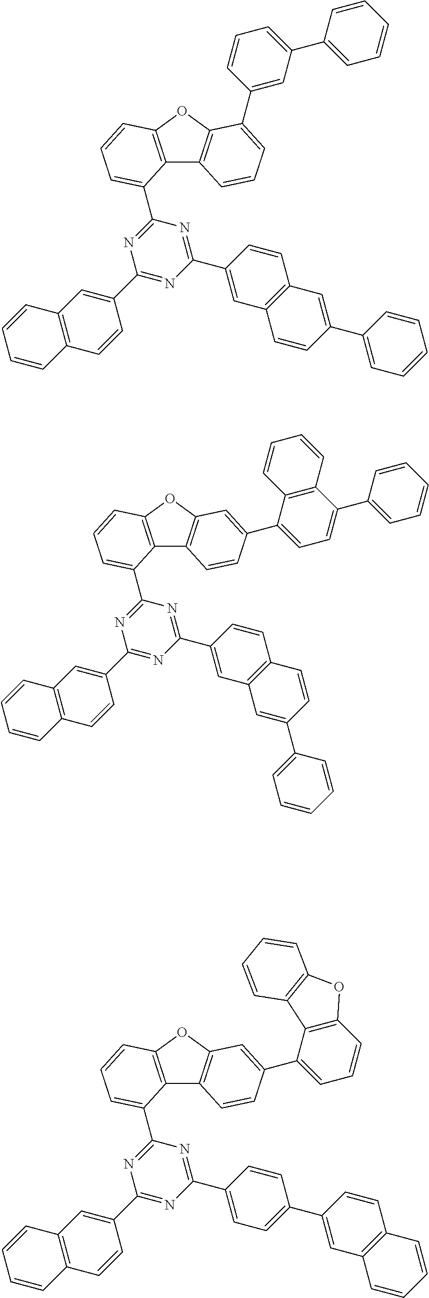 [SMILES: c1ccc(-c2cccc(-c3cccc4c3oc3cccc(-c5nc(-c6ccc7ccccc7c6)nc(-c6ccc7cc(-c8ccccc8)ccc7c6)n5)c34)c2)cc1 ; c1ccc(-c2ccc3ccc(-c4nc(-c5ccc6ccccc6c5)nc(-c5cccc6oc7cc(-c8ccc(-c9ccccc9)c9ccccc89)ccc7c56)n4)cc3c2)cc1 ; c1ccc2cc(-c3ccc(-c4nc(-c5ccc6ccccc6c5)nc(-c5cccc6oc7cc(-c8cccc9oc%10ccccc%10c89)ccc7c56)n4)cc3)ccc2c1]

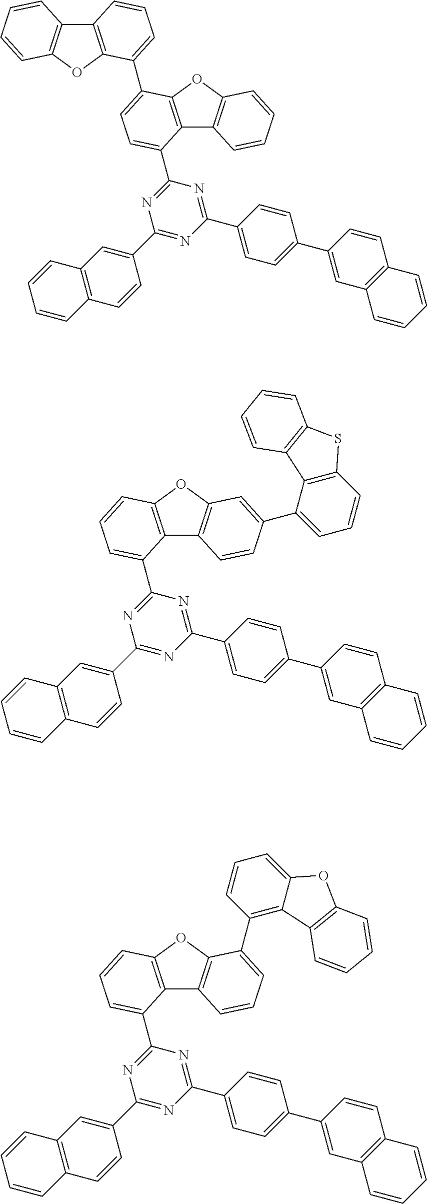 [SMILES: c1ccc2cc(-c3ccc(-c4nc(-c5ccc6ccccc6c5)nc(-c5ccc(-c6cccc7c6oc6ccccc67)c6oc7ccccc7c56)n4)cc3)ccc2c1 ; c1ccc2cc(-c3ccc(-c4nc(-c5ccc6ccccc6c5)nc(-c5cccc6oc7cc(-c8cccc9sc%10ccccc%10c89)ccc7c56)n4)cc3)ccc2c1 ; c1ccc2cc(-c3ccc(-c4nc(-c5ccc6ccccc6c5)nc(-c5cccc6oc7c(-c8cccc9oc%10ccccc%10c89)cccc7c56)n4)cc3)ccc2c1]

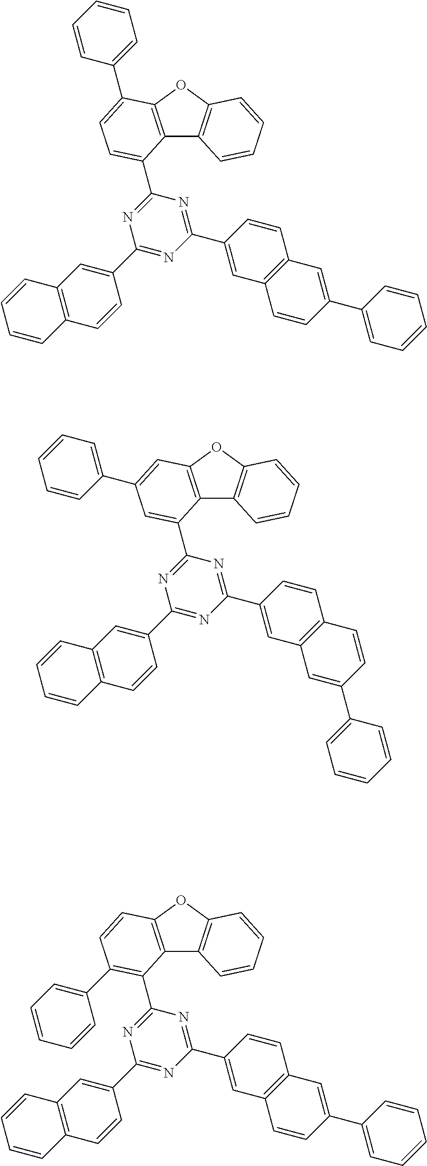 [SMILES: c1ccc(-c2ccc3cc(-c4nc(-c5ccc6ccccc6c5)nc(-c5ccc(-c6ccccc6)c6oc7ccccc7c56)n4)ccc3c2)cc1 ; c1ccc(-c2ccc3ccc(-c4nc(-c5ccc6ccccc6c5)nc(-c5cc(-c6ccccc6)cc6oc7ccccc7c56)n4)cc3c2)cc1 ; c1ccc(-c2ccc3cc(-c4nc(-c5ccc6ccccc6c5)nc(-c5c(-c6ccccc6)ccc6oc7ccccc7c56)n4)ccc3c2)cc1]

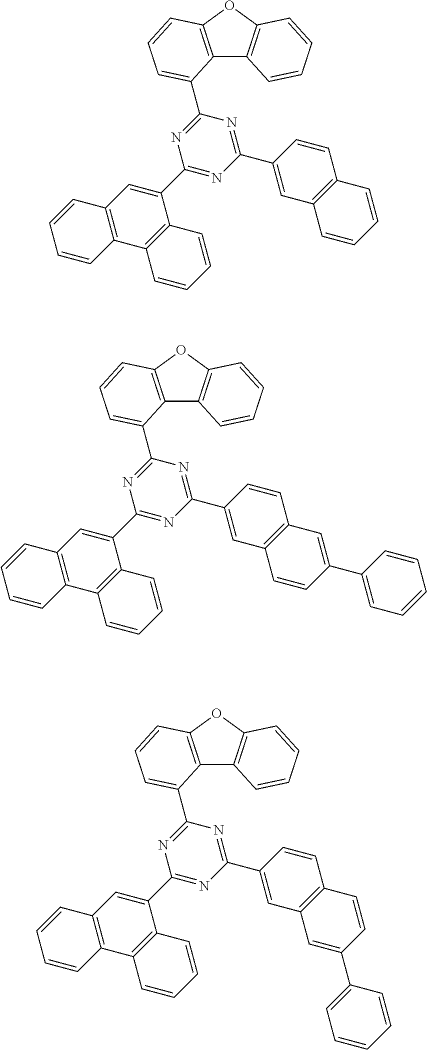 [SMILES: c1ccc2cc(-c3nc(-c4cc5ccccc5c5ccccc45)nc(-c4cccc5oc6ccccc6c45)n3)ccc2c1 ; c1ccc(-c2ccc3cc(-c4nc(-c5cc6ccccc6c6ccccc56)nc(-c5cccc6oc7ccccc7c56)n4)ccc3c2)cc1 ; c1ccc(-c2ccc3ccc(-c4nc(-c5cc6ccccc6c6ccccc56)nc(-c5cccc6oc7ccccc7c56)n4)cc3c2)cc1]

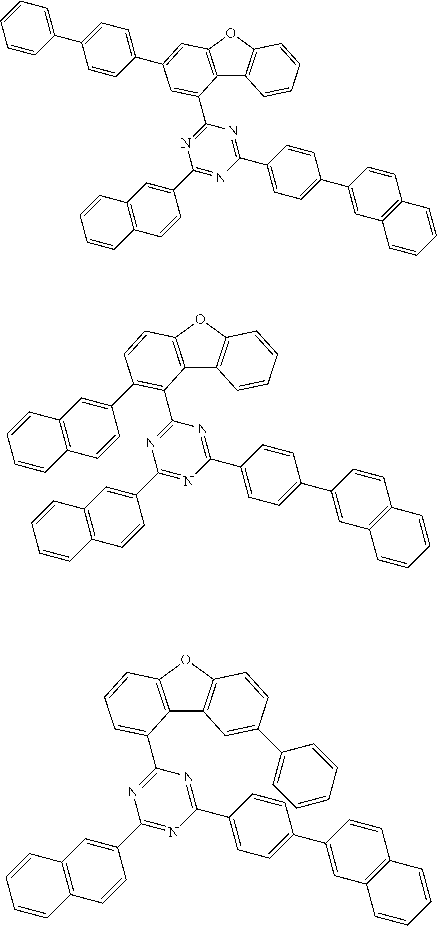 [SMILES: c1ccc(-c2ccc(-c3cc(-c4nc(-c5ccc(-c6ccc7ccccc7c6)cc5)nc(-c5ccc6ccccc6c5)n4)c4c(c3)oc3ccccc34)cc2)cc1 ; c1ccc2cc(-c3ccc(-c4nc(-c5ccc6ccccc6c5)nc(-c5c(-c6ccc7ccccc7c6)ccc6oc7ccccc7c56)n4)cc3)ccc2c1 ; c1ccc(-c2ccc3oc4cccc(-c5nc(-c6ccc(-c7ccc8ccccc8c7)cc6)nc(-c6ccc7ccccc7c6)n5)c4c3c2)cc1]

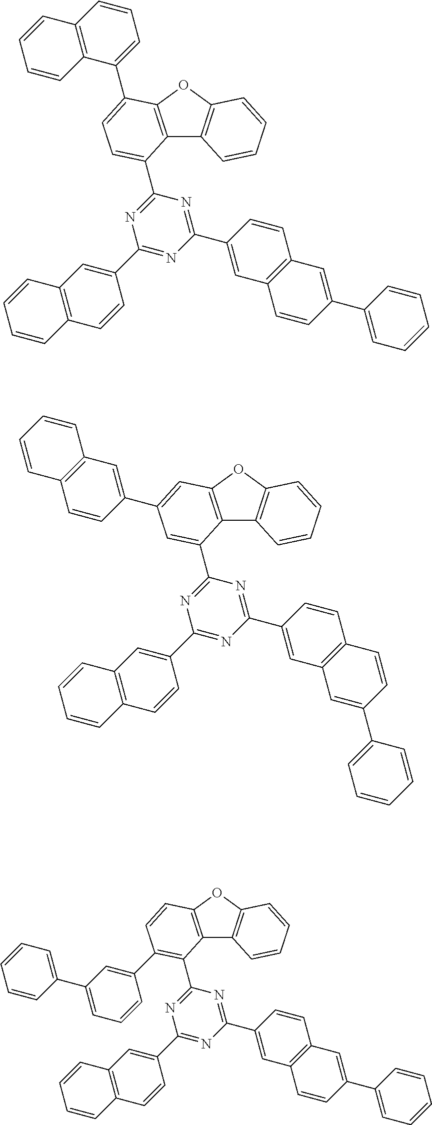 [SMILES: c1ccc(-c2ccc3cc(-c4nc(-c5ccc6ccccc6c5)nc(-c5ccc(-c6cccc7ccccc67)c6oc7ccccc7c56)n4)ccc3c2)cc1 ; c1ccc(-c2ccc3ccc(-c4nc(-c5ccc6ccccc6c5)nc(-c5cc(-c6ccc7ccccc7c6)cc6oc7ccccc7c56)n4)cc3c2)cc1 ; c1ccc(-c2cccc(-c3ccc4oc5ccccc5c4c3-c3nc(-c4ccc5ccccc5c4)nc(-c4ccc5cc(-c6ccccc6)ccc5c4)n3)c2)cc1]

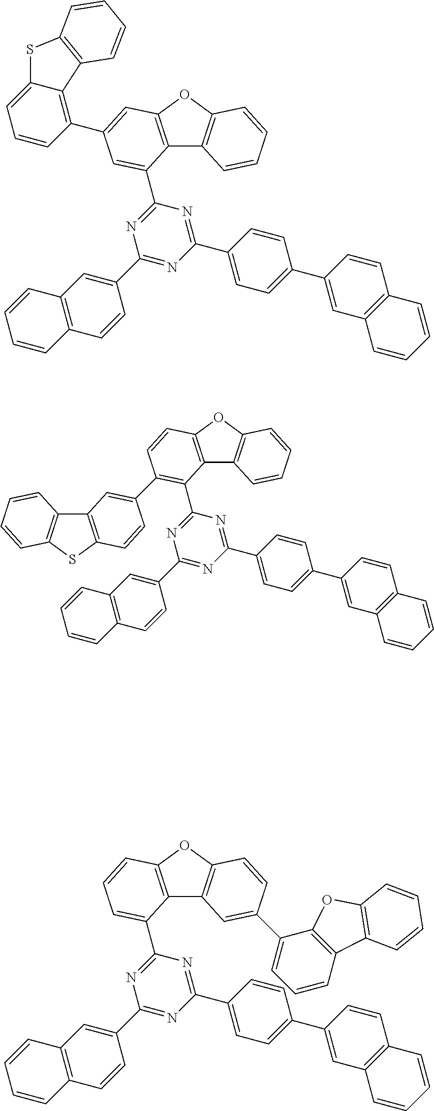 [SMILES: c1ccc2cc(-c3ccc(-c4nc(-c5ccc6ccccc6c5)nc(-c5cc(-c6cccc7sc8ccccc8c67)cc6oc7ccccc7c56)n4)cc3)ccc2c1 ; c1ccc2cc(-c3ccc(-c4nc(-c5ccc6ccccc6c5)nc(-c5c(-c6ccc7sc8ccccc8c7c6)ccc6oc7ccccc7c56)n4)cc3)ccc2c1 ; c1ccc2cc(-c3ccc(-c4nc(-c5ccc6ccccc6c5)nc(-c5cccc6oc7ccc(-c8cccc9c8oc8ccccc89)cc7c56)n4)cc3)ccc2c1]

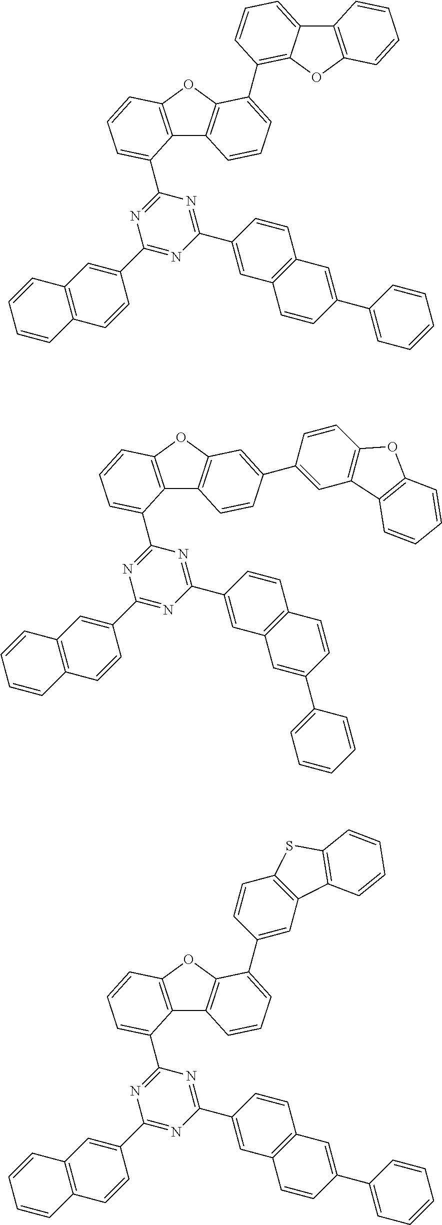 [SMILES: c1ccc(-c2ccc3cc(-c4nc(-c5ccc6ccccc6c5)nc(-c5cccc6oc7c(-c8cccc9c8oc8ccccc89)cccc7c56)n4)ccc3c2)cc1 ; c1ccc(-c2ccc3ccc(-c4nc(-c5ccc6ccccc6c5)nc(-c5cccc6oc7cc(-c8ccc9oc%10ccccc%10c9c8)ccc7c56)n4)cc3c2)cc1 ; c1ccc(-c2ccc3cc(-c4nc(-c5ccc6ccccc6c5)nc(-c5cccc6oc7c(-c8ccc9sc%10ccccc%10c9c8)cccc7c56)n4)ccc3c2)cc1]

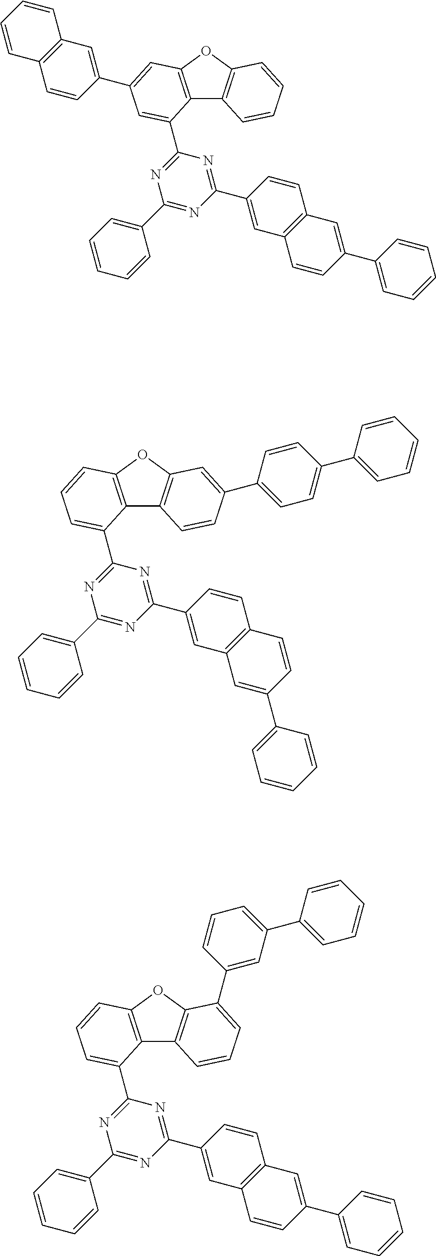 [SMILES: c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2ccc(-c3ccc4c(c3)oc3cccc(-c5nc(-c6ccccc6)nc(-c6ccc7ccc(-c8ccccc8)cc7c6)n5)c34)cc2)cc1 ; c1ccc(-c2cccc(-c3cccc4c3oc3cccc(-c5nc(-c6ccccc6)nc(-c6ccc7cc(-c8ccccc8)ccc7c6)n5)c34)c2)cc1]

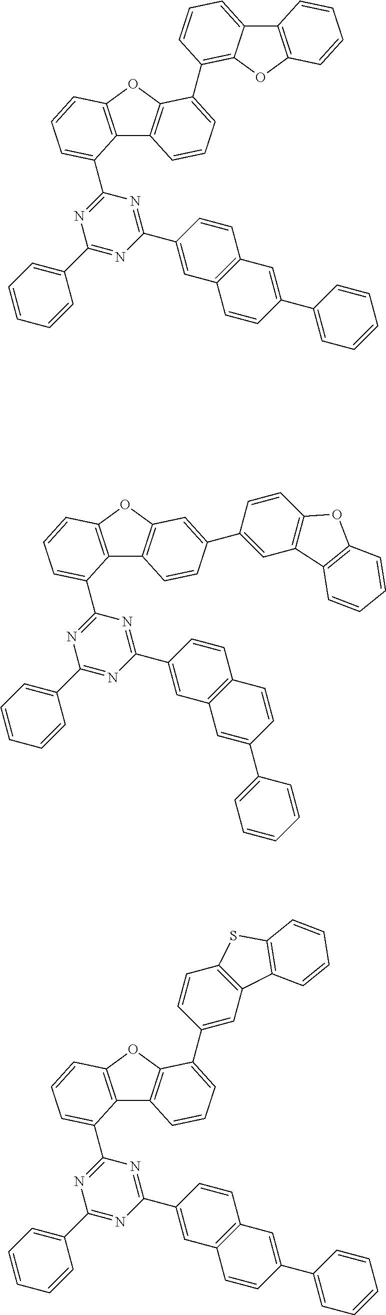 [SMILES: c1ccc(-c2ccc3cc(-c4nc(-c5ccccc5)nc(-c5cccc6oc7c(-c8cccc9c8oc8ccccc89)cccc7c56)n4)ccc3c2)cc1 ; c1ccc(-c2ccc3ccc(-c4nc(-c5ccccc5)nc(-c5cccc6oc7cc(-c8ccc9oc%10ccccc%10c9c8)ccc7c56)n4)cc3c2)cc1 ; c1ccc(-c2ccc3cc(-c4nc(-c5ccccc5)nc(-c5cccc6oc7c(-c8ccc9sc%10ccccc%10c9c8)cccc7c56)n4)ccc3c2)cc1]

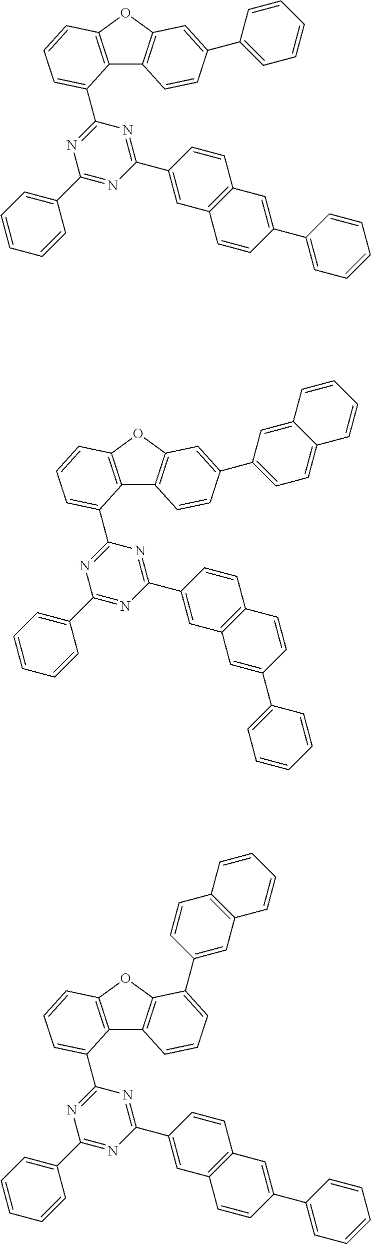 [SMILES: c1ccc(-c2ccc3cc(-c4nc(-c5ccccc5)nc(-c5cccc6oc7cc(-c8ccccc8)ccc7c56)n4)ccc3c2)cc1 ; c1ccc(-c2ccc3ccc(-c4nc(-c5ccccc5)nc(-c5cccc6oc7cc(-c8ccc9ccccc9c8)ccc7c56)n4)cc3c2)cc1 ; c1ccc(-c2ccc3cc(-c4nc(-c5ccccc5)nc(-c5cccc6oc7c(-c8ccc9ccccc9c8)cccc7c56)n4)ccc3c2)cc1]

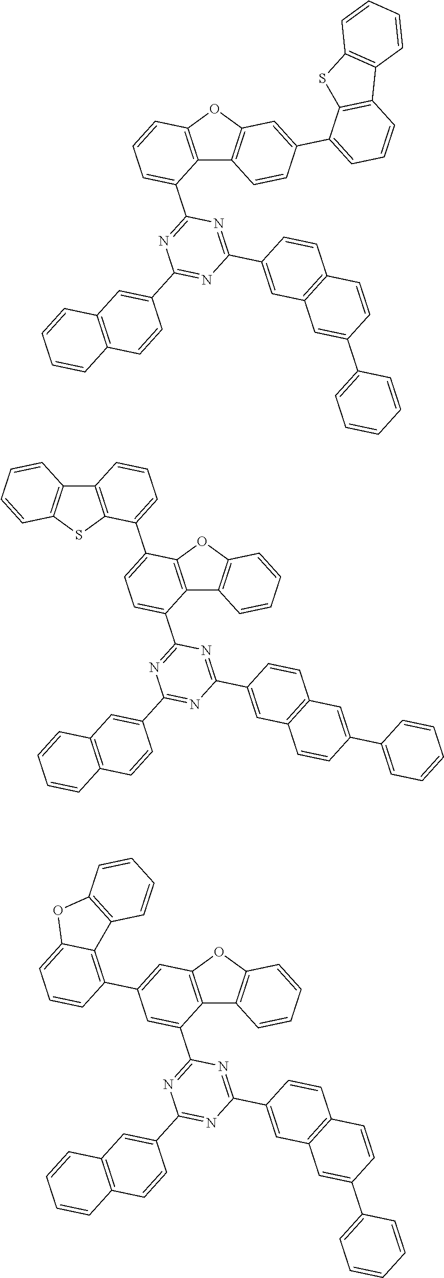 [SMILES: c1ccc(-c2ccc3ccc(-c4nc(-c5ccc6ccccc6c5)nc(-c5cccc6oc7cc(-c8cccc9c8sc8ccccc89)ccc7c56)n4)cc3c2)cc1 ; c1ccc(-c2ccc3cc(-c4nc(-c5ccc6ccccc6c5)nc(-c5ccc(-c6cccc7c6sc6ccccc67)c6oc7ccccc7c56)n4)ccc3c2)cc1 ; c1ccc(-c2ccc3ccc(-c4nc(-c5ccc6ccccc6c5)nc(-c5cc(-c6cccc7oc8ccccc8c67)cc6oc7ccccc7c56)n4)cc3c2)cc1]

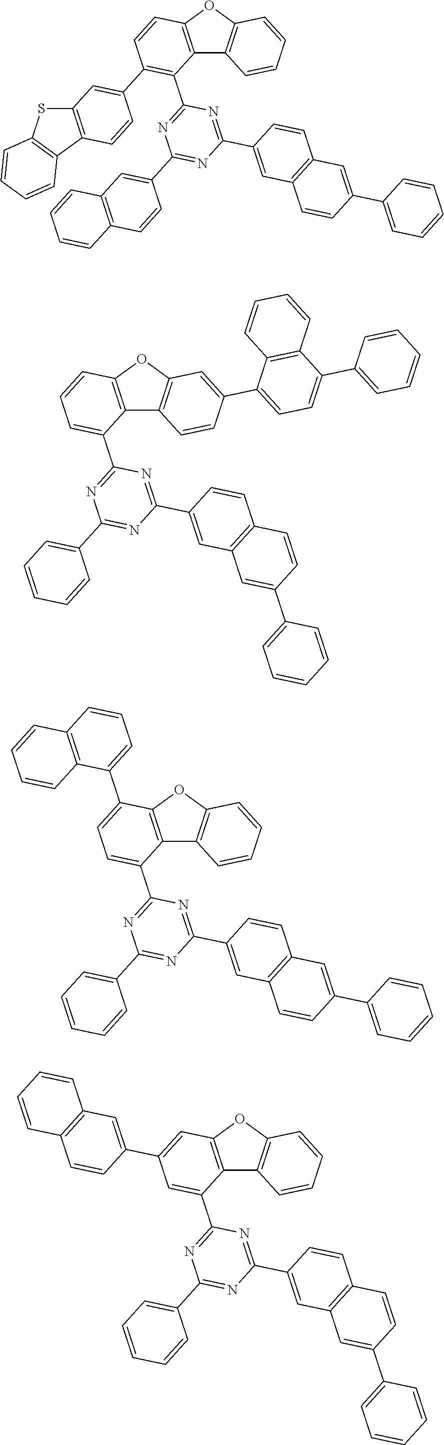 [SMILES: c1ccc(-c2ccc3cc(-c4nc(-c5ccc6ccccc6c5)nc(-c5c(-c6ccc7c(c6)sc6ccccc67)ccc6oc7ccccc7c56)n4)ccc3c2)cc1 ; c1ccc(-c2ccc3ccc(-c4nc(-c5ccccc5)nc(-c5cccc6oc7cc(-c8ccc(-c9ccccc9)c9ccccc89)ccc7c56)n4)cc3c2)cc1 ; c1ccc(-c2ccc3cc(-c4nc(-c5ccccc5)nc(-c5ccc(-c6cccc7ccccc67)c6oc7ccccc7c56)n4)ccc3c2)cc1 ; c1ccc(-c2ccc3ccc(-c4nc(-c5ccccc5)nc(-c5cc(-c6ccc7ccccc7c6)cc6oc7ccccc7c56)n4)cc3c2)cc1]

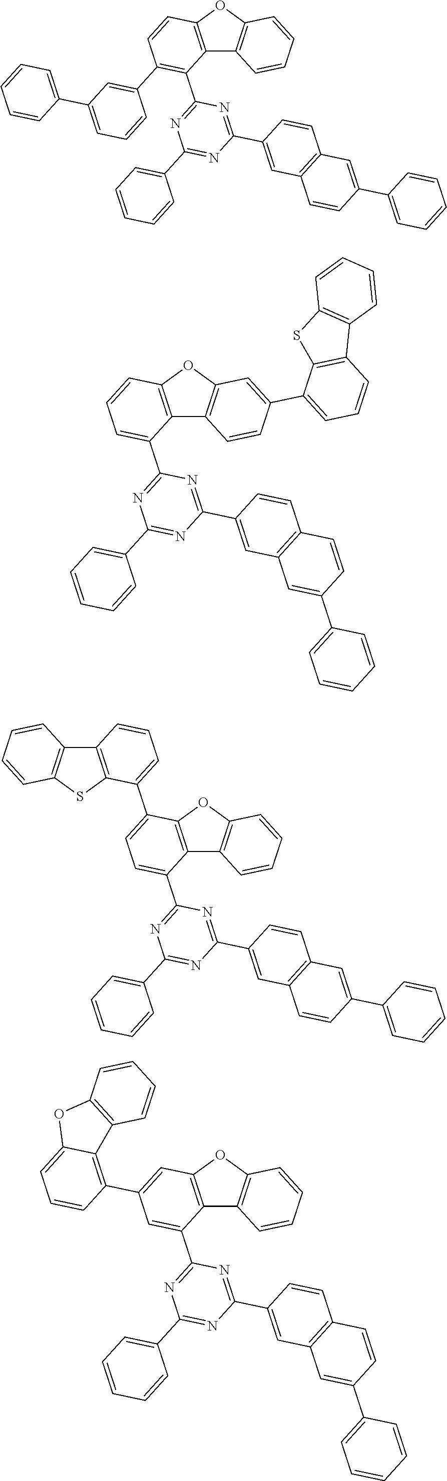

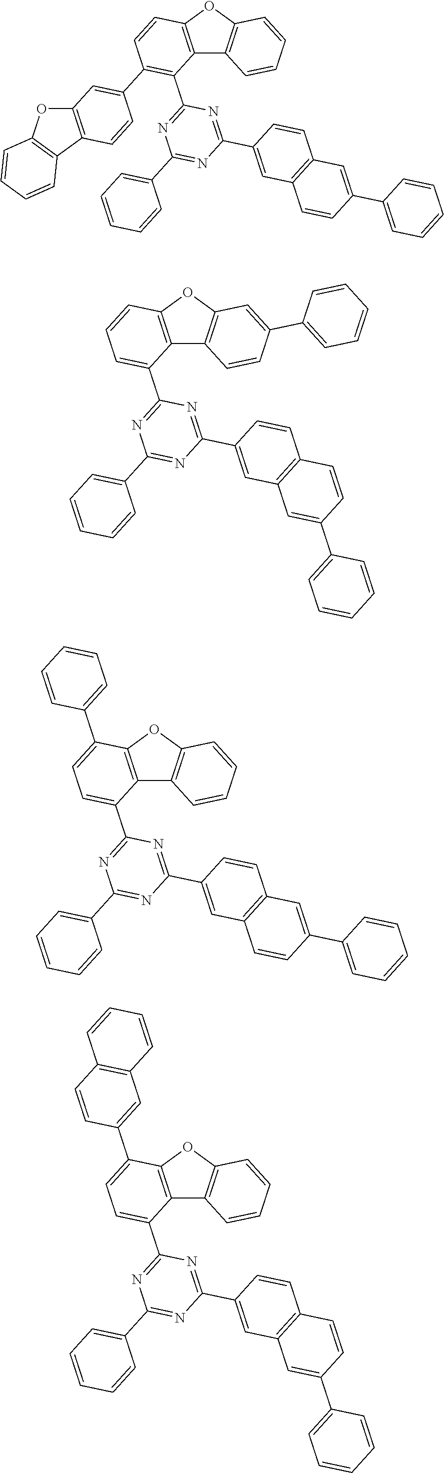

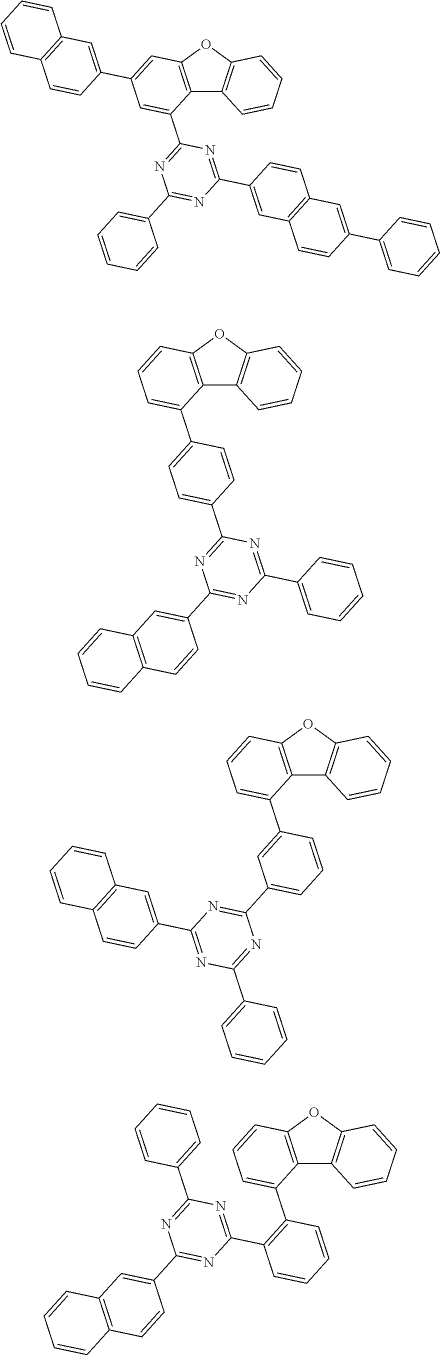 [SMILES: c1ccc(-c2ccc3cc(-c4nc(-c5ccccc5)nc(-c5cc(-c6ccc7ccccc7c6)cc6oc7ccccc7c56)n4)ccc3c2)cc1 ; c1ccc(-c2nc(-c3ccc(-c4cccc5oc6ccccc6c45)cc3)nc(-c3ccc4ccccc4c3)n2)cc1 ; c1ccc(-c2nc(-c3cccc(-c4cccc5oc6ccccc6c45)c3)nc(-c3ccc4ccccc4c3)n2)cc1 ; c1ccc(-c2nc(-c3ccc4ccccc4c3)nc(-c3ccccc3-c3cccc4oc5ccccc5c34)n2)cc1]

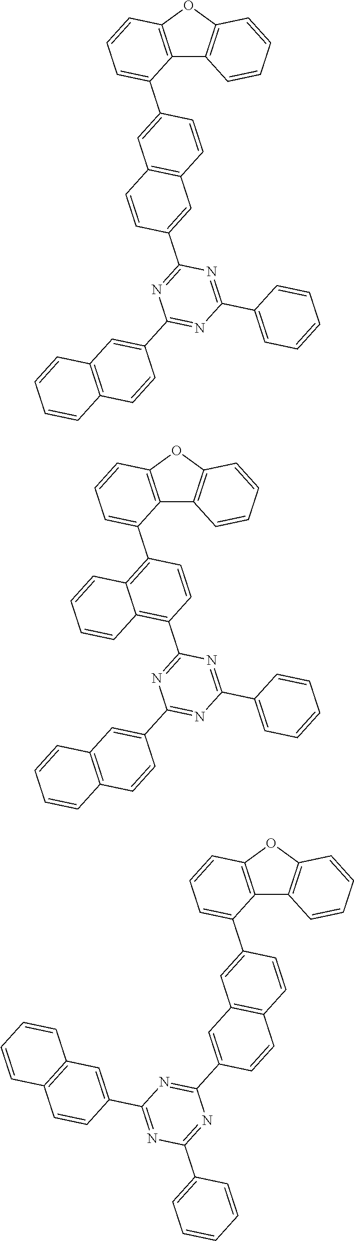 [SMILES: c1ccc(-c2nc(-c3ccc4ccccc4c3)nc(-c3ccc4cc(-c5cccc6oc7ccccc7c56)ccc4c3)n2)cc1 ; c1ccc(-c2nc(-c3ccc4ccccc4c3)nc(-c3ccc(-c4cccc5oc6ccccc6c45)c4ccccc34)n2)cc1 ; c1ccc(-c2nc(-c3ccc4ccccc4c3)nc(-c3ccc4ccc(-c5cccc6oc7ccccc7c56)cc4c3)n2)cc1]

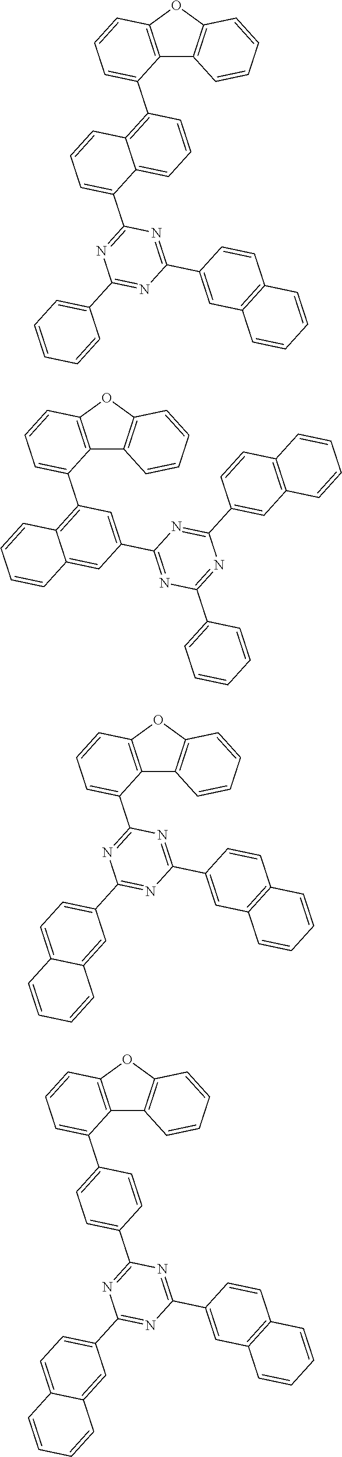

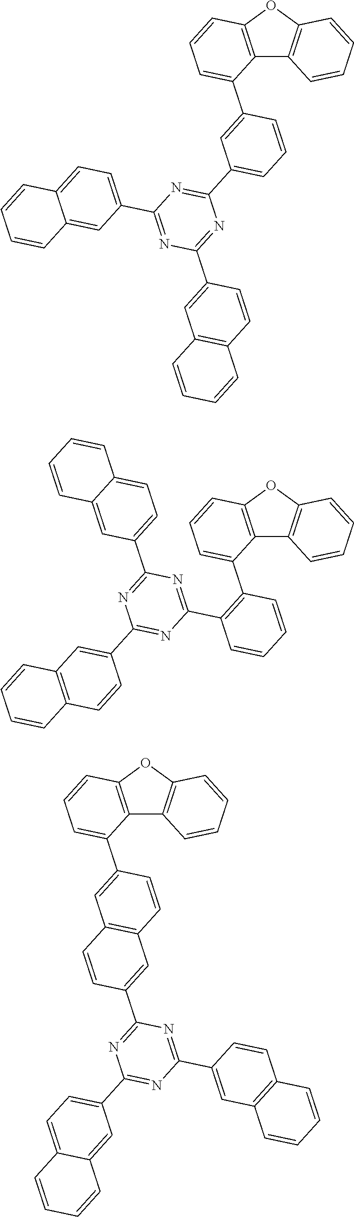 [SMILES: c1cc(-c2nc(-c3ccc4ccccc4c3)nc(-c3ccc4ccccc4c3)n2)cc(-c2cccc3oc4ccccc4c23)c1 ; c1ccc(-c2cccc3oc4ccccc4c23)c(-c2nc(-c3ccc4ccccc4c3)nc(-c3ccc4ccccc4c3)n2)c1 ; c1ccc2cc(-c3nc(-c4ccc5ccccc5c4)nc(-c4ccc5cc(-c6cccc7oc8ccccc8c67)ccc5c4)n3)ccc2c1]

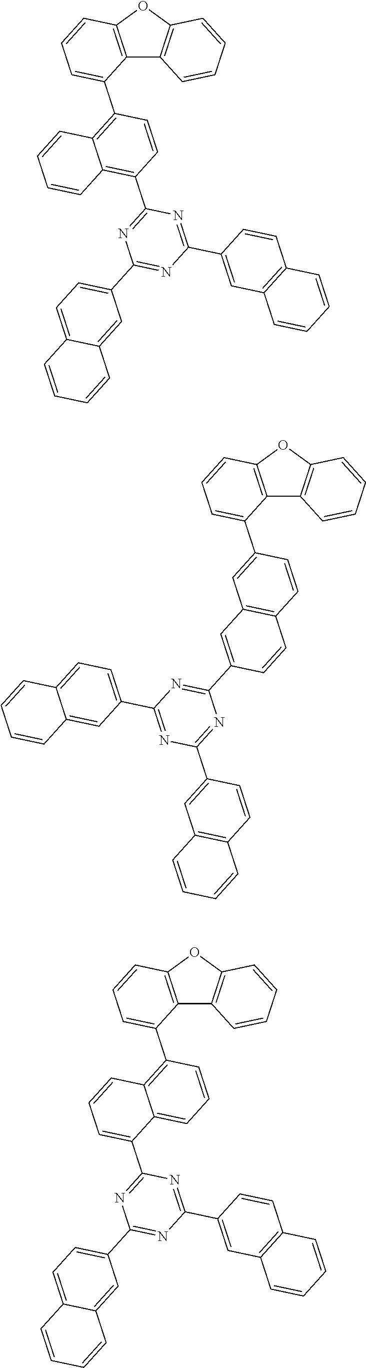 [SMILES: c1ccc2cc(-c3nc(-c4ccc5ccccc5c4)nc(-c4ccc(-c5cccc6oc7ccccc7c56)c5ccccc45)n3)ccc2c1 ; c1ccc2cc(-c3nc(-c4ccc5ccccc5c4)nc(-c4ccc5ccc(-c6cccc7oc8ccccc8c67)cc5c4)n3)ccc2c1 ; c1ccc2cc(-c3nc(-c4ccc5ccccc5c4)nc(-c4cccc5c(-c6cccc7oc8ccccc8c67)cccc45)n3)ccc2c1]

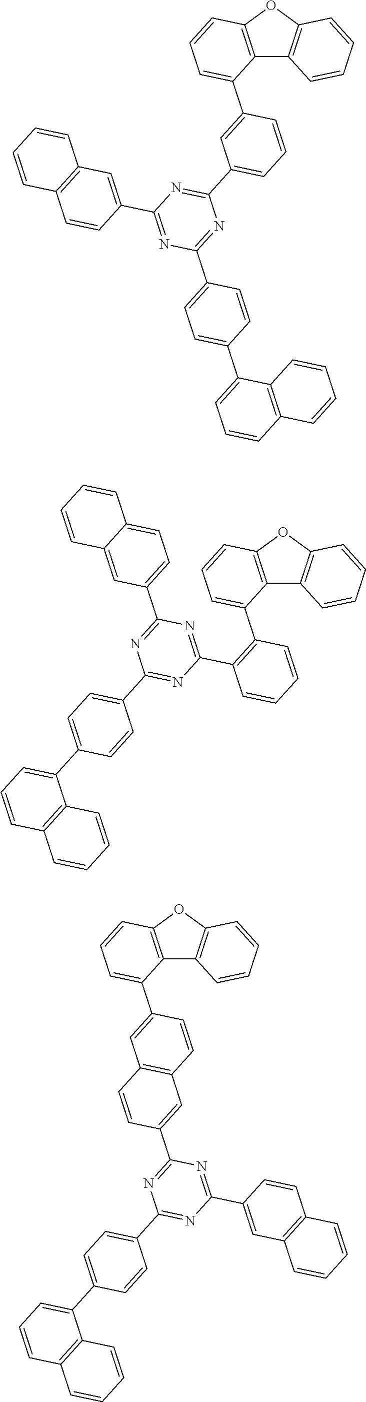 [SMILES: c1cc(-c2nc(-c3ccc(-c4cccc5ccccc45)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4cccc5ccccc45)cc3)nc(-c3ccc4ccccc4c3)n2)c1 ; c1ccc2cc(-c3nc(-c4ccc(-c5cccc6ccccc56)cc4)nc(-c4ccc5cc(-c6cccc7oc8ccccc8c67)ccc5c4)n3)ccc2c1]

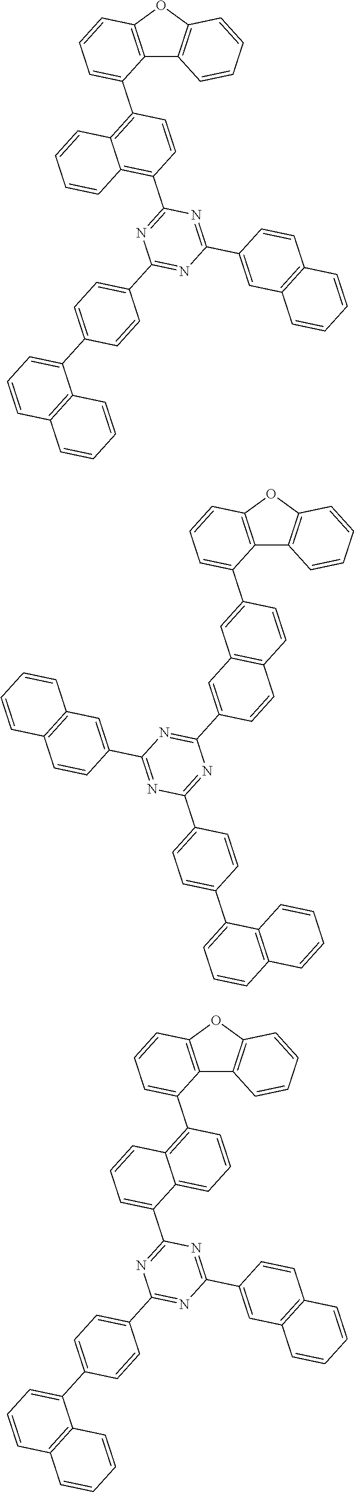

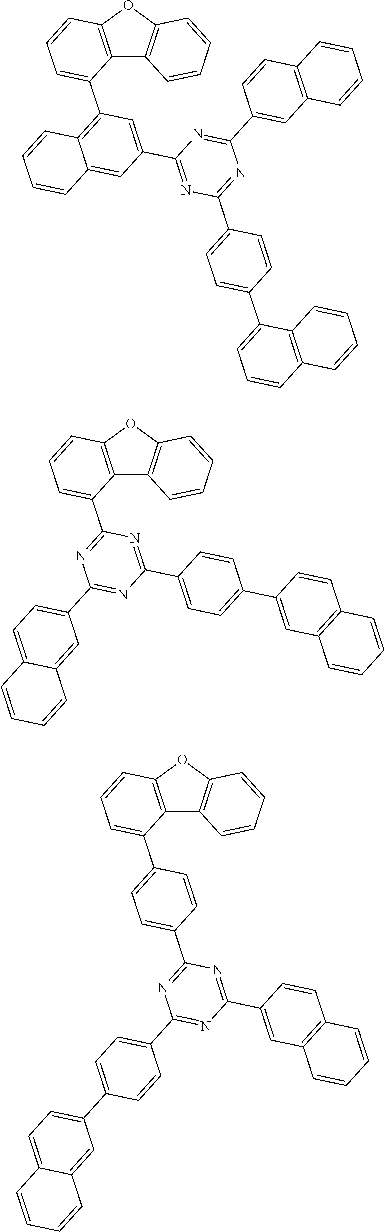 [SMILES: c1ccc2cc(-c3nc(-c4ccc(-c5cccc6ccccc56)cc4)nc(-c4cc(-c5cccc6oc7ccccc7c56)c5ccccc5c4)n3)ccc2c1 ; c1ccc2cc(-c3ccc(-c4nc(-c5ccc6ccccc6c5)nc(-c5cccc6oc7ccccc7c56)n4)cc3)ccc2c1 ; c1ccc2cc(-c3ccc(-c4nc(-c5ccc(-c6cccc7oc8ccccc8c67)cc5)nc(-c5ccc6ccccc6c5)n4)cc3)ccc2c1]

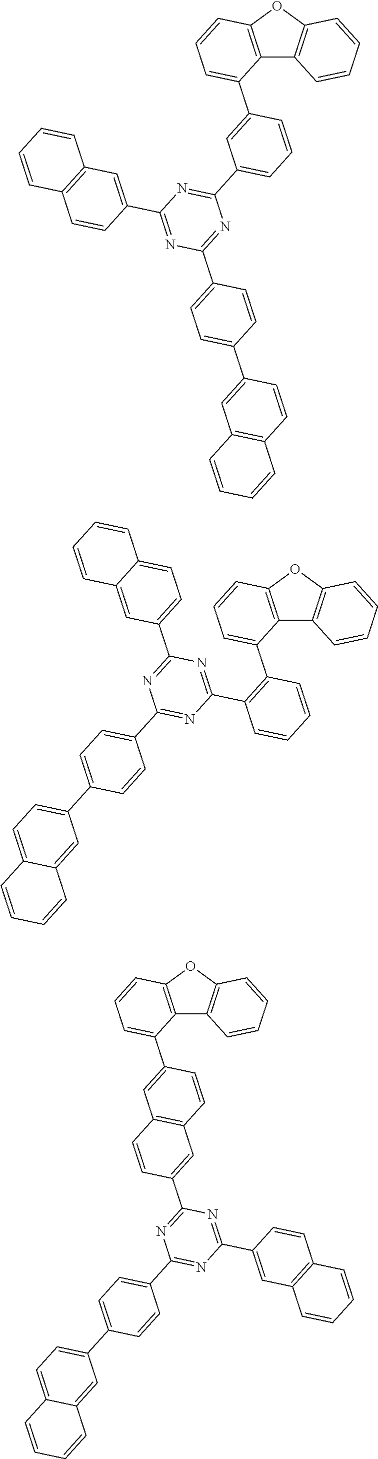 [SMILES: c1cc(-c2nc(-c3ccc(-c4ccc5ccccc5c4)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4ccc5ccccc5c4)cc3)nc(-c3ccc4ccccc4c3)n2)c1 ; c1ccc2cc(-c3ccc(-c4nc(-c5ccc6ccccc6c5)nc(-c5ccc6cc(-c7cccc8oc9ccccc9c78)ccc6c5)n4)cc3)ccc2c1]

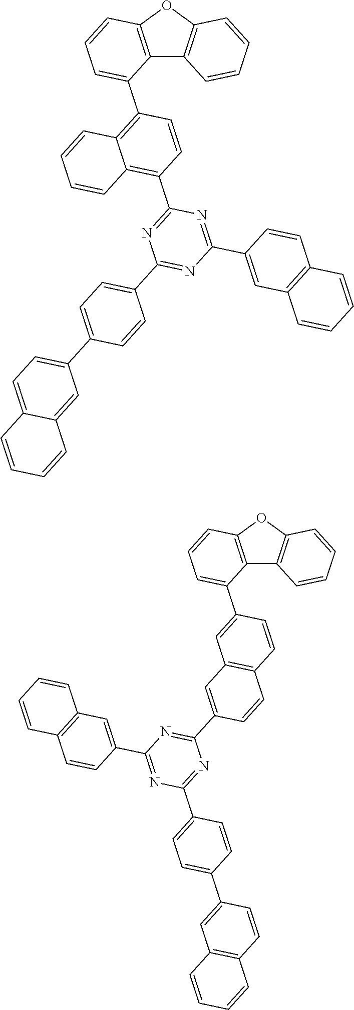 [SMILES: c1ccc2cc(-c3ccc(-c4nc(-c5ccc6ccccc6c5)nc(-c5ccc(-c6cccc7oc8ccccc8c67)c6ccccc56)n4)cc3)ccc2c1 ; c1ccc2cc(-c3ccc(-c4nc(-c5ccc6ccccc6c5)nc(-c5ccc6ccc(-c7cccc8oc9ccccc9c78)cc6c5)n4)cc3)ccc2c1]

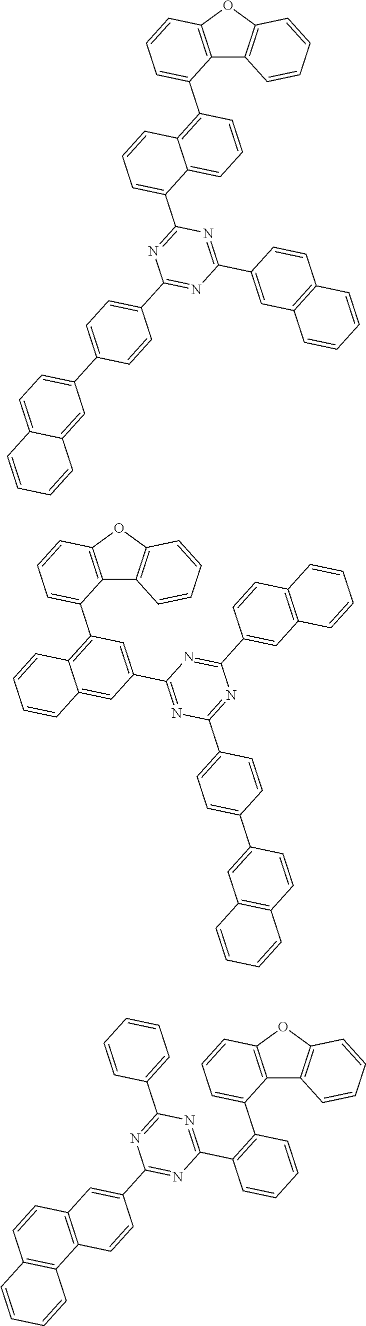

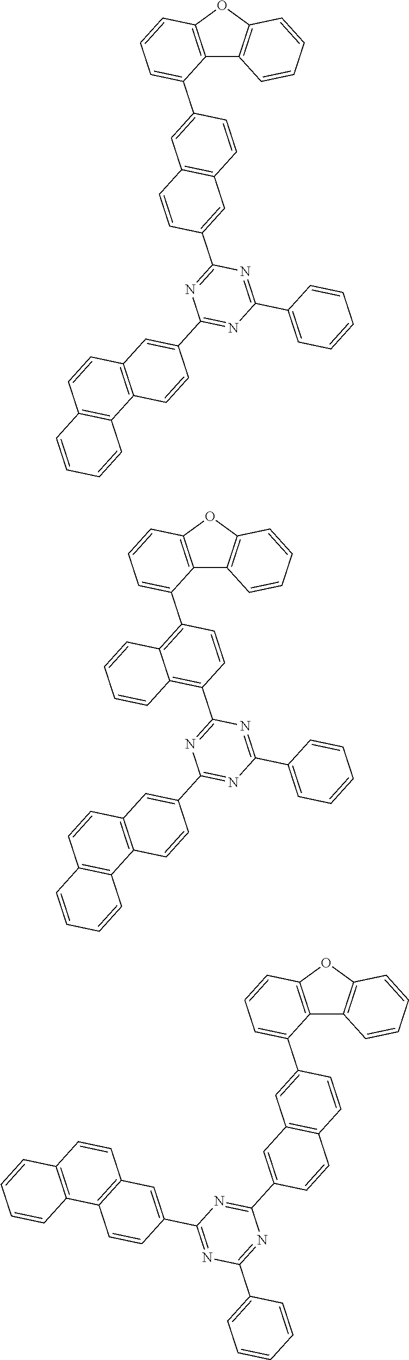 [SMILES: c1ccc(-c2nc(-c3ccc4cc(-c5cccc6oc7ccccc7c56)ccc4c3)nc(-c3ccc4c(ccc5ccccc54)c3)n2)cc1 ; c1ccc(-c2nc(-c3ccc4c(ccc5ccccc54)c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(ccc5ccccc54)c3)n2)cc1]

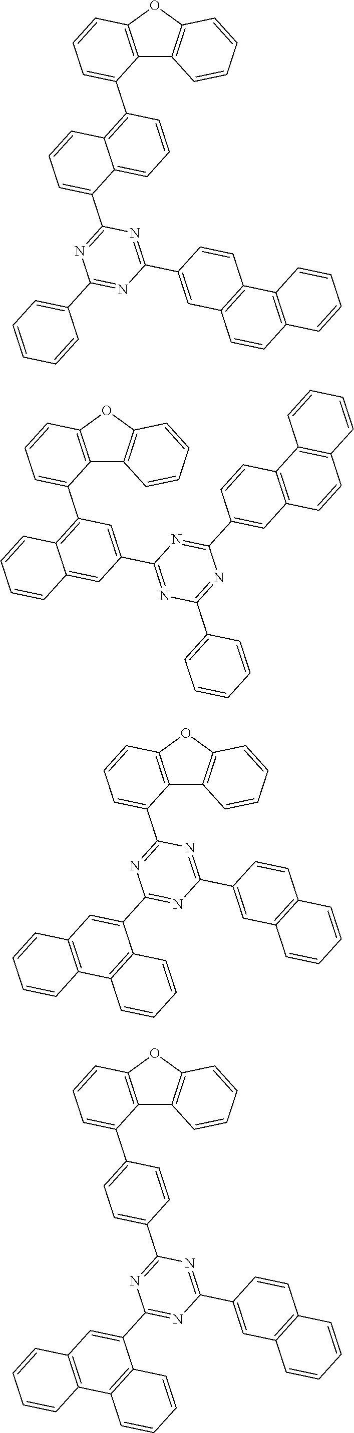

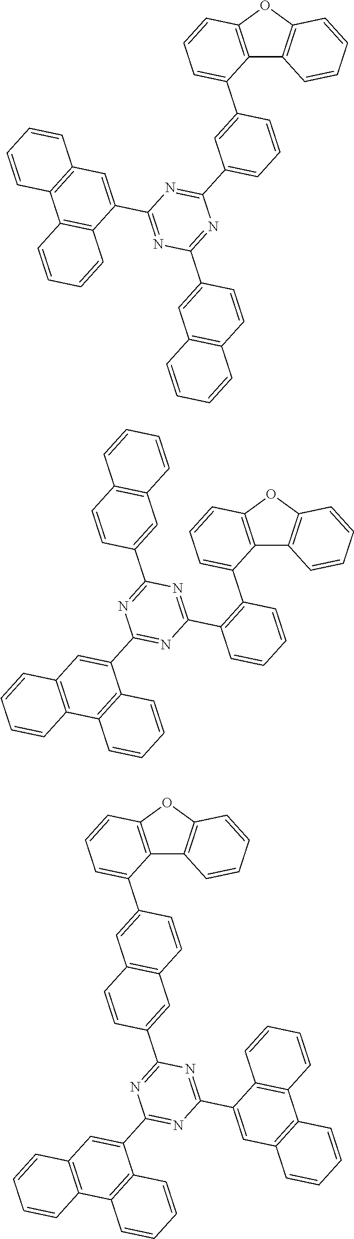

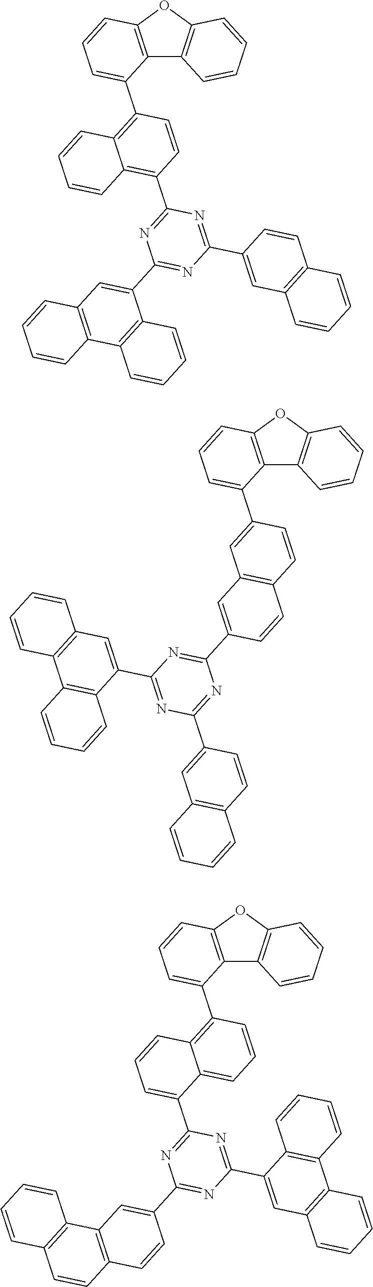 [SMILES: c1ccc2cc(-c3nc(-c4ccc(-c5cccc6oc7ccccc7c56)c5ccccc45)nc(-c4cc5ccccc5c5ccccc45)n3)ccc2c1 ; c1ccc2cc(-c3nc(-c4ccc5ccc(-c6cccc7oc8ccccc8c67)cc5c4)nc(-c4cc5ccccc5c5ccccc45)n3)ccc2c1 ; c1ccc2c(c1)ccc1ccc(-c3nc(-c4cccc5c(-c6cccc7oc8ccccc8c67)cccc45)nc(-c4cc5ccccc5c5ccccc45)n3)cc12]

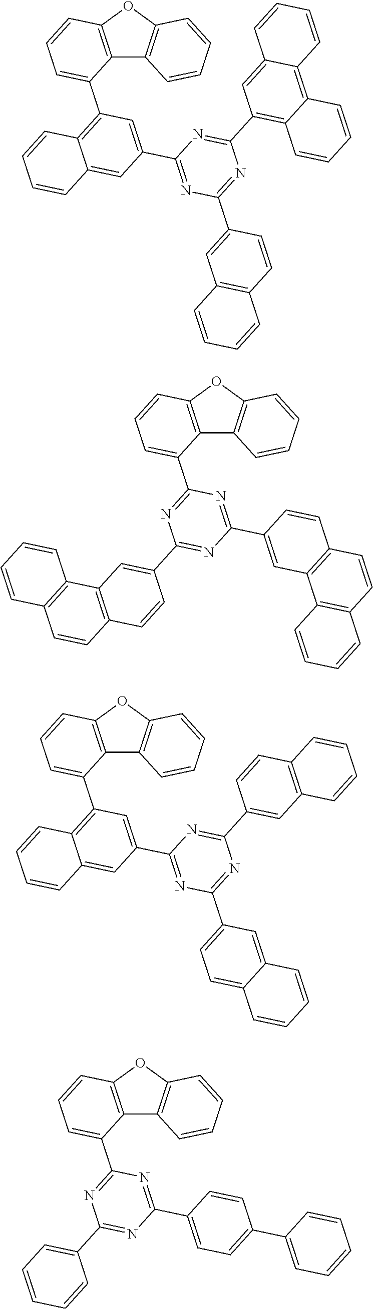

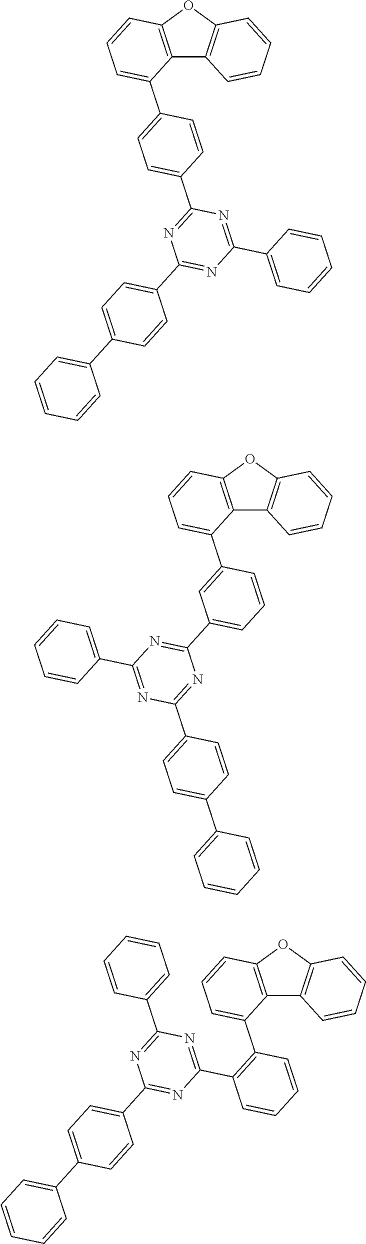 [SMILES: c1ccc(-c2ccc(-c3nc(-c4ccccc4)nc(-c4ccc(-c5cccc6oc7ccccc7c56)cc4)n3)cc2)cc1 ; c1ccc(-c2ccc(-c3nc(-c4ccccc4)nc(-c4cccc(-c5cccc6oc7ccccc7c56)c4)n3)cc2)cc1 ; c1ccc(-c2ccc(-c3nc(-c4ccccc4)nc(-c4ccccc4-c4cccc5oc6ccccc6c45)n3)cc2)cc1]

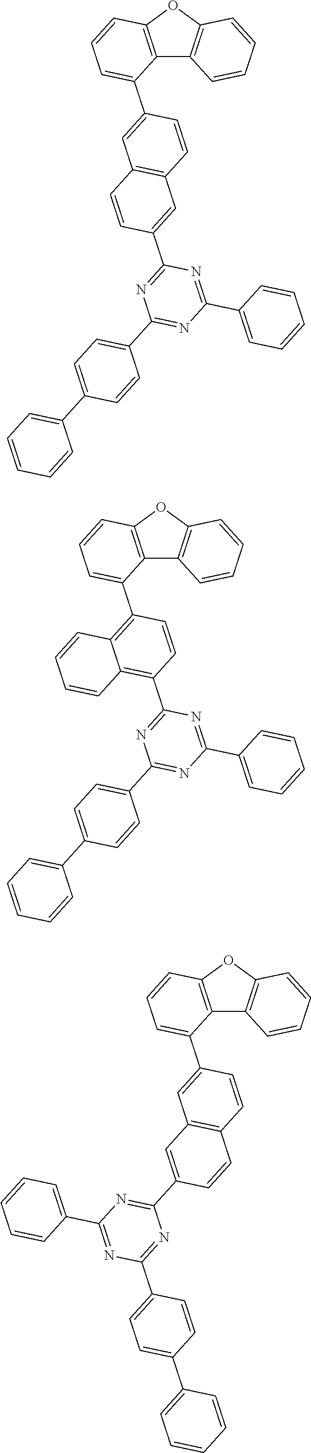 [SMILES: c1ccc(-c2ccc(-c3nc(-c4ccccc4)nc(-c4ccc5cc(-c6cccc7oc8ccccc8c67)ccc5c4)n3)cc2)cc1 ; c1ccc(-c2ccc(-c3nc(-c4ccccc4)nc(-c4ccc(-c5cccc6oc7ccccc7c56)c5ccccc45)n3)cc2)cc1 ; c1ccc(-c2ccc(-c3nc(-c4ccccc4)nc(-c4ccc5ccc(-c6cccc7oc8ccccc8c67)cc5c4)n3)cc2)cc1]

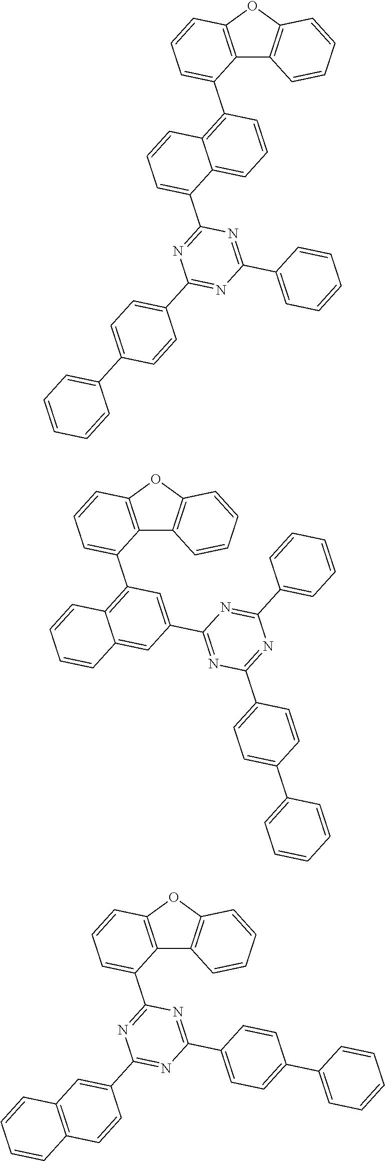 [SMILES: c1ccc(-c2ccc(-c3nc(-c4ccccc4)nc(-c4cccc5c(-c6cccc7oc8ccccc8c67)cccc45)n3)cc2)cc1 ; c1ccc(-c2ccc(-c3nc(-c4ccccc4)nc(-c4cc(-c5cccc6oc7ccccc7c56)c5ccccc5c4)n3)cc2)cc1 ; c1ccc(-c2ccc(-c3nc(-c4ccc5ccccc5c4)nc(-c4cccc5oc6ccccc6c45)n3)cc2)cc1]

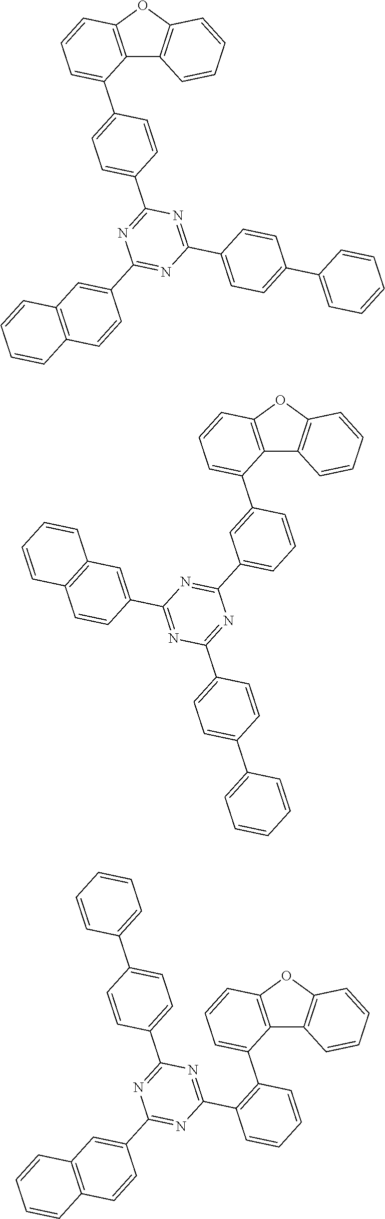 [SMILES: c1ccc(-c2ccc(-c3nc(-c4ccc(-c5cccc6oc7ccccc7c56)cc4)nc(-c4ccc5ccccc5c4)n3)cc2)cc1 ; c1ccc(-c2ccc(-c3nc(-c4cccc(-c5cccc6oc7ccccc7c56)c4)nc(-c4ccc5ccccc5c4)n3)cc2)cc1 ; c1ccc(-c2ccc(-c3nc(-c4ccc5ccccc5c4)nc(-c4ccccc4-c4cccc5oc6ccccc6c45)n3)cc2)cc1]

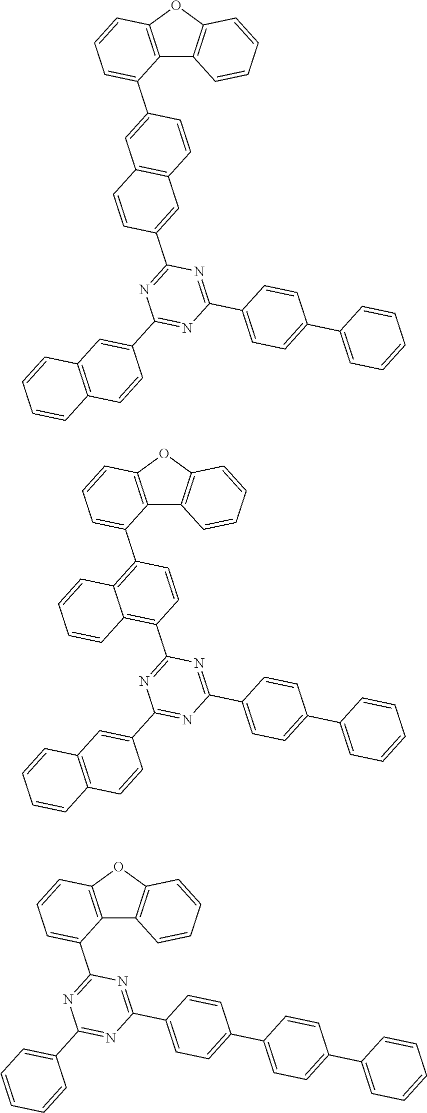 [SMILES: c1ccc(-c2ccc(-c3nc(-c4ccc5ccccc5c4)nc(-c4ccc5cc(-c6cccc7oc8ccccc8c67)ccc5c4)n3)cc2)cc1 ; c1ccc(-c2ccc(-c3nc(-c4ccc5ccccc5c4)nc(-c4ccc(-c5cccc6oc7ccccc7c56)c5ccccc45)n3)cc2)cc1 ; c1ccc(-c2ccc(-c3ccc(-c4nc(-c5ccccc5)nc(-c5cccc6oc7ccccc7c56)n4)cc3)cc2)cc1]

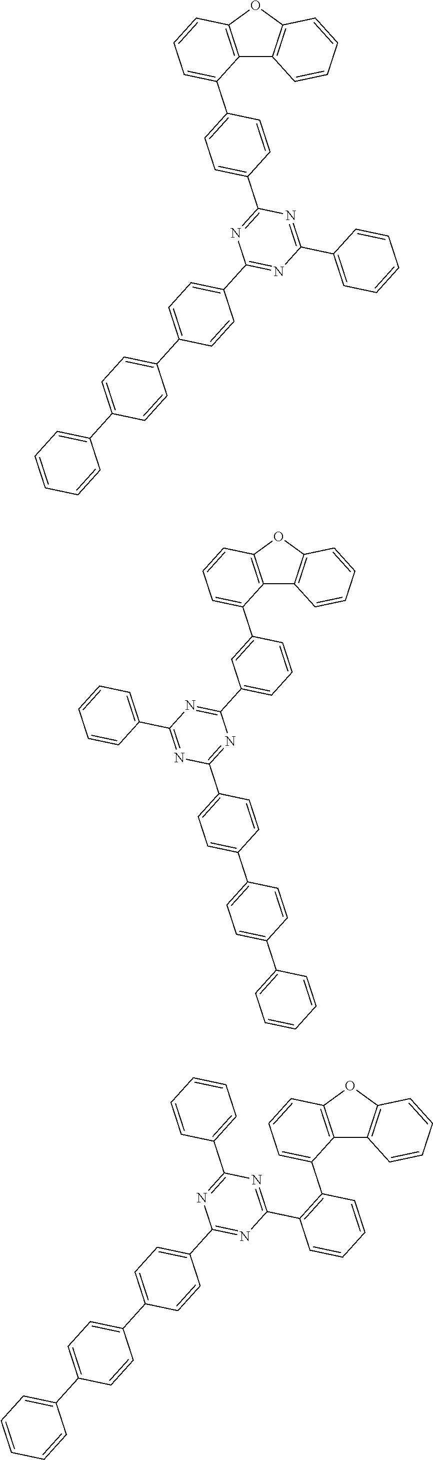 [SMILES: c1ccc(-c2ccc(-c3ccc(-c4nc(-c5ccccc5)nc(-c5ccc(-c6cccc7oc8ccccc8c67)cc5)n4)cc3)cc2)cc1 ; c1ccc(-c2ccc(-c3ccc(-c4nc(-c5ccccc5)nc(-c5cccc(-c6cccc7oc8ccccc8c67)c5)n4)cc3)cc2)cc1 ; c1ccc(-c2ccc(-c3ccc(-c4nc(-c5ccccc5)nc(-c5ccccc5-c5cccc6oc7ccccc7c56)n4)cc3)cc2)cc1]

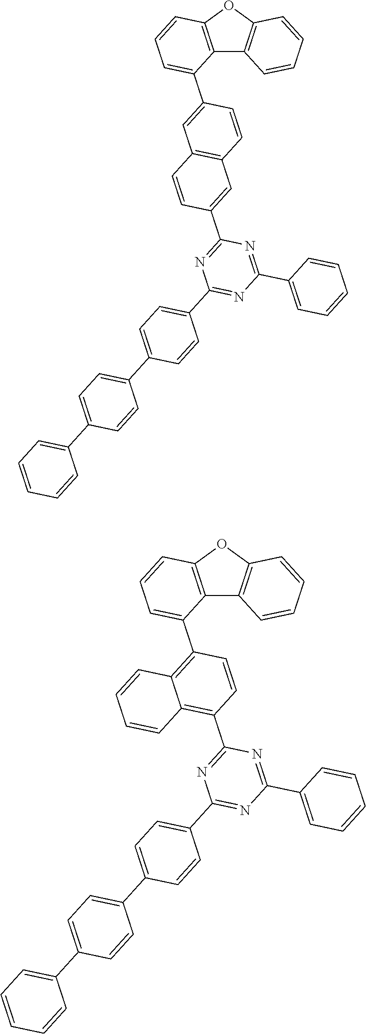 [SMILES: c1ccc(-c2ccc(-c3ccc(-c4nc(-c5ccccc5)nc(-c5ccc6cc(-c7cccc8oc9ccccc9c78)ccc6c5)n4)cc3)cc2)cc1 ; c1ccc(-c2ccc(-c3ccc(-c4nc(-c5ccccc5)nc(-c5ccc(-c6cccc7oc8ccccc8c67)c6ccccc56)n4)cc3)cc2)cc1]

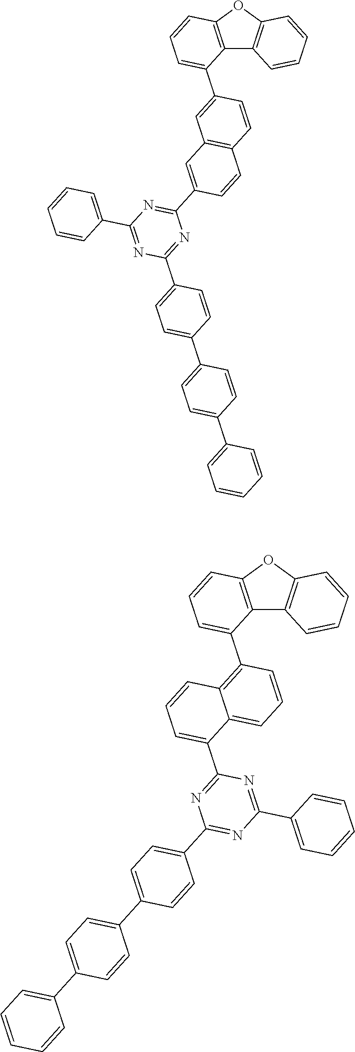 [SMILES: c1ccc(-c2ccc(-c3ccc(-c4nc(-c5ccccc5)nc(-c5ccc6ccc(-c7cccc8oc9ccccc9c78)cc6c5)n4)cc3)cc2)cc1 ; c1ccc(-c2ccc(-c3ccc(-c4nc(-c5ccccc5)nc(-c5cccc6c(-c7cccc8oc9ccccc9c78)cccc56)n4)cc3)cc2)cc1]

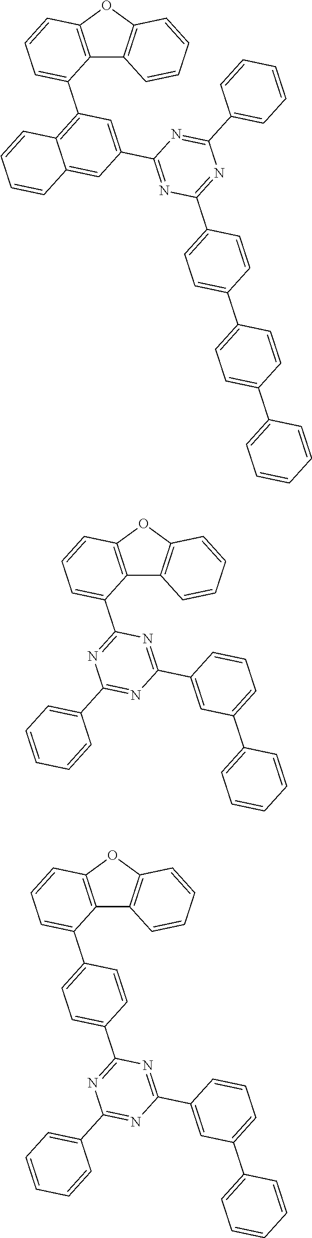 [SMILES: c1ccc(-c2ccc(-c3ccc(-c4nc(-c5ccccc5)nc(-c5cc(-c6cccc7oc8ccccc8c67)c6ccccc6c5)n4)cc3)cc2)cc1 ; c1ccc(-c2cccc(-c3nc(-c4ccccc4)nc(-c4cccc5oc6ccccc6c45)n3)c2)cc1 ; c1ccc(-c2cccc(-c3nc(-c4ccccc4)nc(-c4ccc(-c5cccc6oc7ccccc7c56)cc4)n3)c2)cc1]

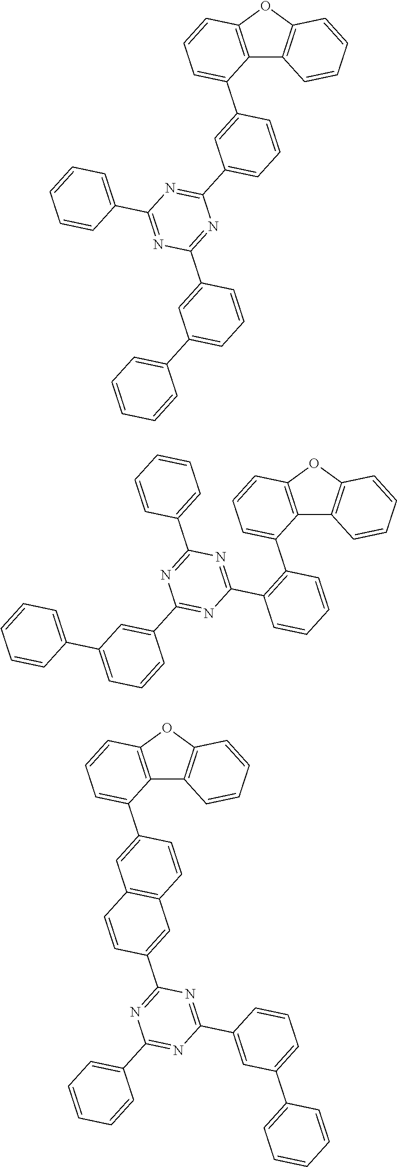

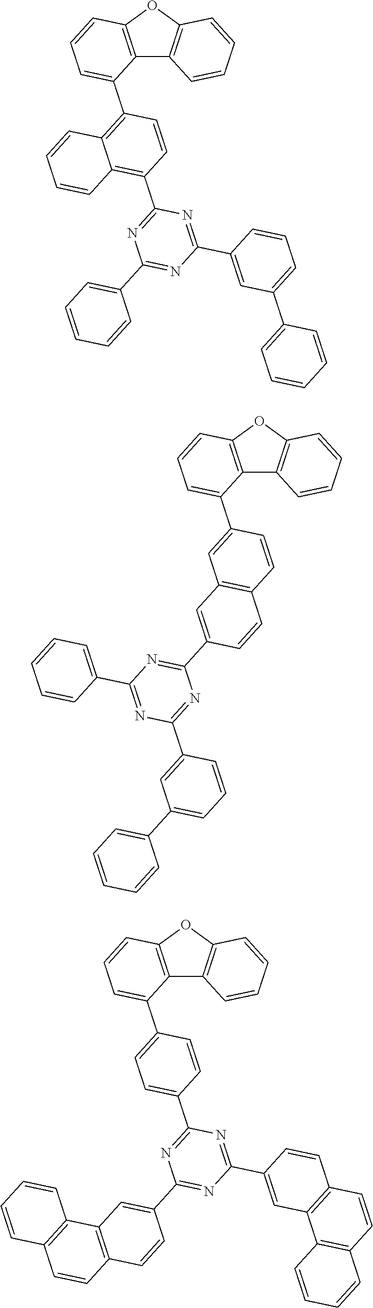

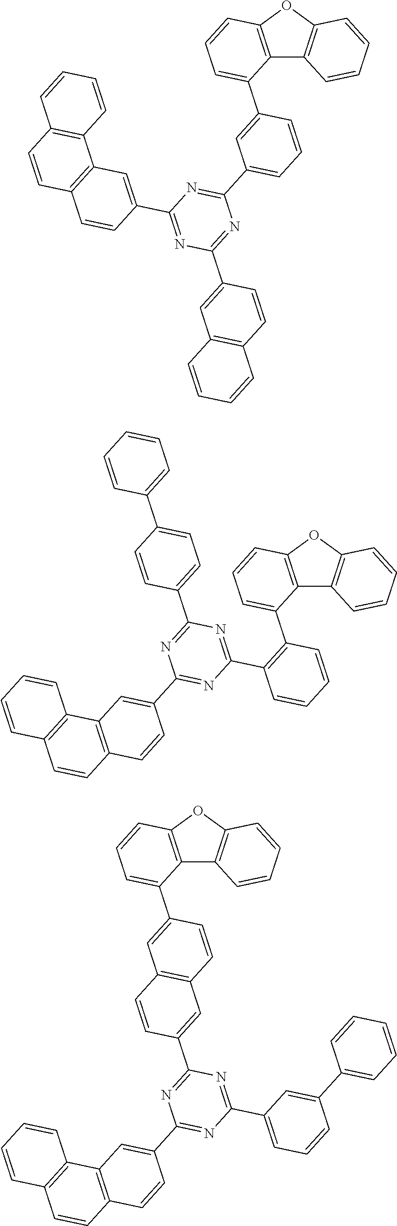 [SMILES: c1cc(-c2nc(-c3ccc4ccccc4c3)nc(-c3ccc4ccc5ccccc5c4c3)n2)cc(-c2cccc3oc4ccccc4c23)c1 ; c1ccc(-c2ccc(-c3nc(-c4ccc5ccc6ccccc6c5c4)nc(-c4ccccc4-c4cccc5oc6ccccc6c45)n3)cc2)cc1 ; c1ccc(-c2cccc(-c3nc(-c4ccc5cc(-c6cccc7oc8ccccc8c67)ccc5c4)nc(-c4ccc5ccc6ccccc6c5c4)n3)c2)cc1]

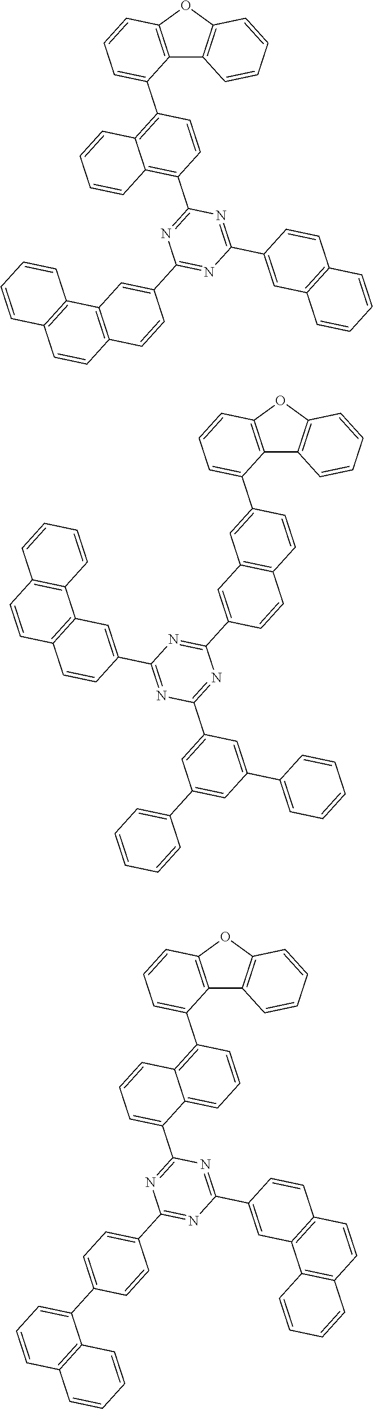 [SMILES: c1ccc2cc(-c3nc(-c4ccc5ccc6ccccc6c5c4)nc(-c4ccc(-c5cccc6oc7ccccc7c56)c5ccccc45)n3)ccc2c1 ; c1ccc(-c2cc(-c3ccccc3)cc(-c3nc(-c4ccc5ccc(-c6cccc7oc8ccccc8c67)cc5c4)nc(-c4ccc5ccc6ccccc6c5c4)n3)c2)cc1 ; c1ccc2c(-c3ccc(-c4nc(-c5ccc6ccc7ccccc7c6c5)nc(-c5cccc6c(-c7cccc8oc9ccccc9c78)cccc56)n4)cc3)cccc2c1]

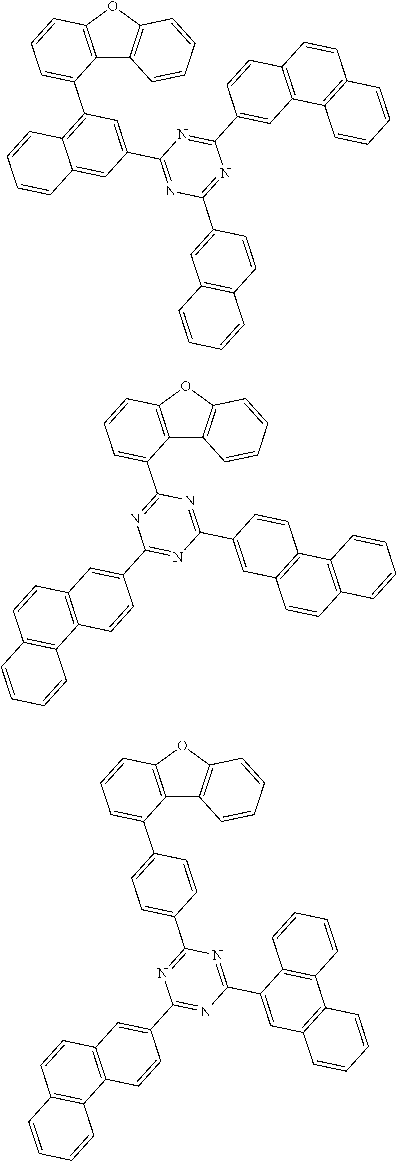

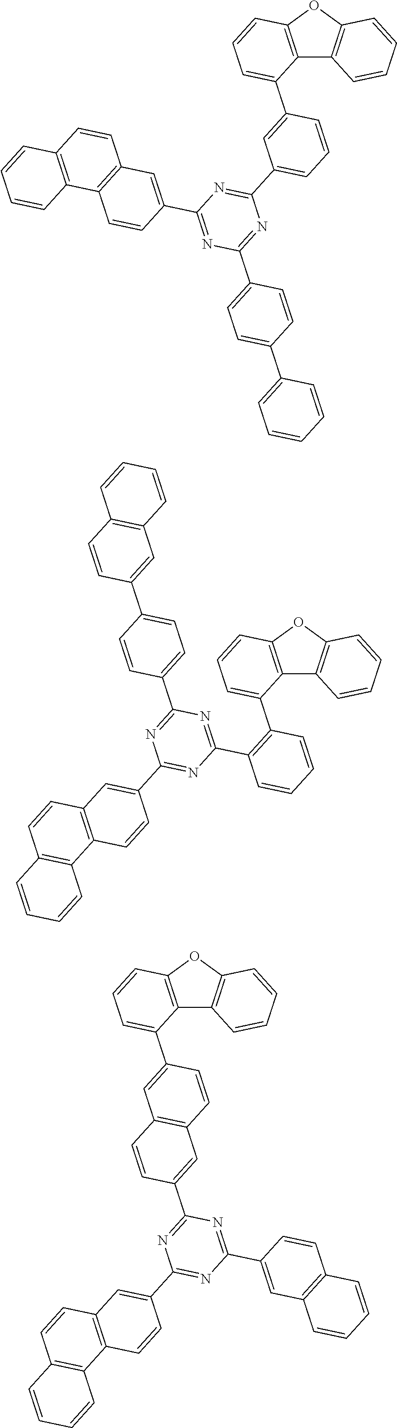 [SMILES: c1ccc(-c2ccc(-c3nc(-c4cccc(-c5cccc6oc7ccccc7c56)c4)nc(-c4ccc5c(ccc6ccccc65)c4)n3)cc2)cc1 ; c1ccc(-c2cccc3oc4ccccc4c23)c(-c2nc(-c3ccc(-c4ccc5ccccc5c4)cc3)nc(-c3ccc4c(ccc5ccccc54)c3)n2)c1 ; c1ccc2cc(-c3nc(-c4ccc5cc(-c6cccc7oc8ccccc8c67)ccc5c4)nc(-c4ccc5c(ccc6ccccc65)c4)n3)ccc2c1]

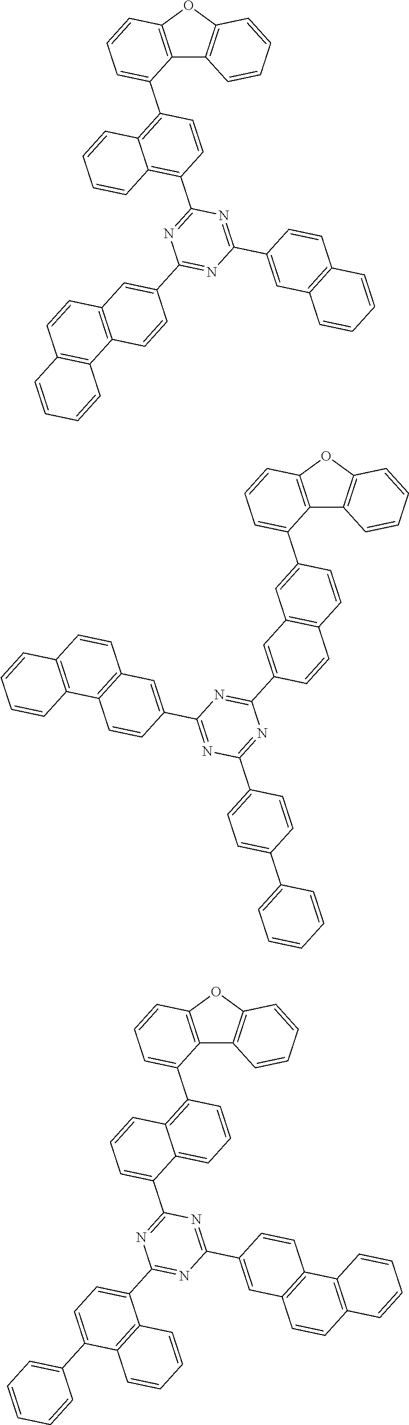 [SMILES: c1ccc2cc(-c3nc(-c4ccc5c(ccc6ccccc65)c4)nc(-c4ccc(-c5cccc6oc7ccccc7c56)c5ccccc45)n3)ccc2c1 ; c1ccc(-c2ccc(-c3nc(-c4ccc5ccc(-c6cccc7oc8ccccc8c67)cc5c4)nc(-c4ccc5c(ccc6ccccc65)c4)n3)cc2)cc1 ; c1ccc(-c2ccc(-c3nc(-c4ccc5c(ccc6ccccc65)c4)nc(-c4cccc5c(-c6cccc7oc8ccccc8c67)cccc45)n3)c3ccccc23)cc1]

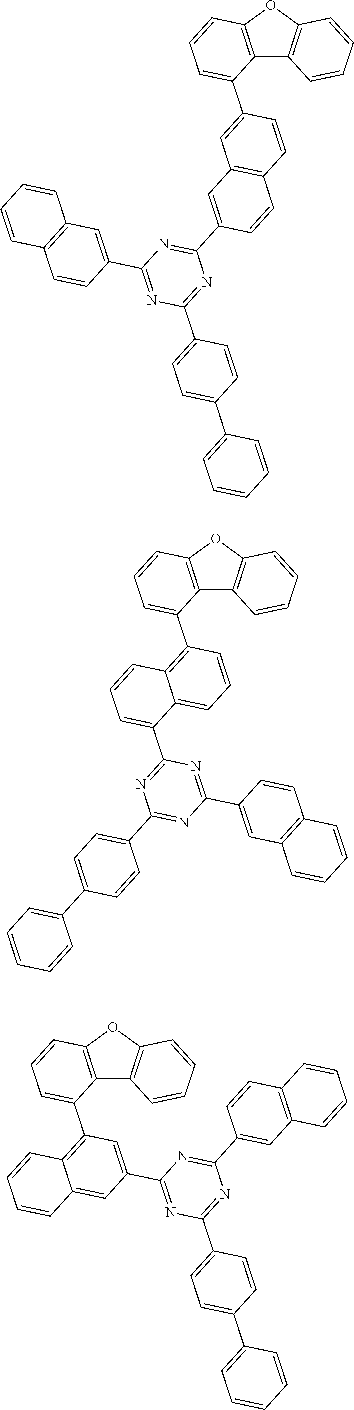 [SMILES: c1ccc(-c2ccc(-c3nc(-c4ccc5ccccc5c4)nc(-c4ccc5ccc(-c6cccc7oc8ccccc8c67)cc5c4)n3)cc2)cc1 ; c1ccc(-c2ccc(-c3nc(-c4ccc5ccccc5c4)nc(-c4cccc5c(-c6cccc7oc8ccccc8c67)cccc45)n3)cc2)cc1 ; c1ccc(-c2ccc(-c3nc(-c4ccc5ccccc5c4)nc(-c4cc(-c5cccc6oc7ccccc7c56)c5ccccc5c4)n3)cc2)cc1]

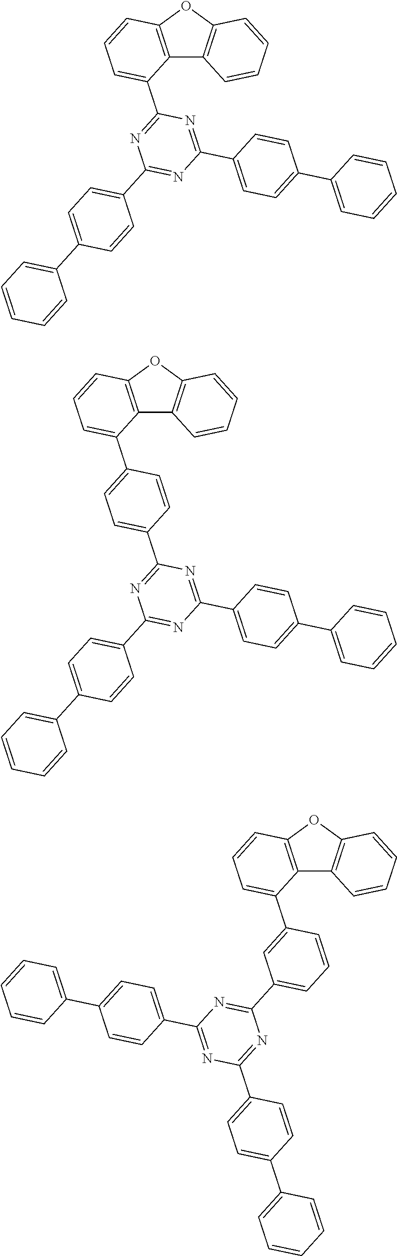 [SMILES: c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4ccc(-c5ccccc5)cc4)nc(-c4ccc(-c5cccc6oc7ccccc7c56)cc4)n3)cc2)cc1 ; c1ccc(-c2ccc(-c3nc(-c4ccc(-c5ccccc5)cc4)nc(-c4cccc(-c5cccc6oc7ccccc7c56)c4)n3)cc2)cc1]

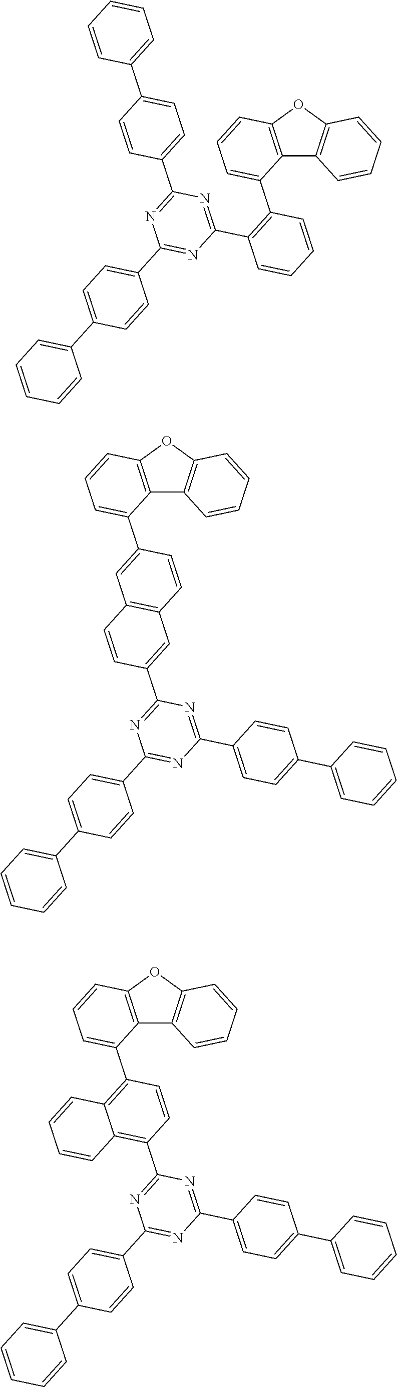 [SMILES: c1ccc(-c2ccc(-c3nc(-c4ccc(-c5ccccc5)cc4)nc(-c4ccccc4-c4cccc5oc6ccccc6c45)n3)cc2)cc1 ; c1ccc(-c2ccc(-c3nc(-c4ccc(-c5ccccc5)cc4)nc(-c4ccc5cc(-c6cccc7oc8ccccc8c67)ccc5c4)n3)cc2)cc1 ; c1ccc(-c2ccc(-c3nc(-c4ccc(-c5ccccc5)cc4)nc(-c4ccc(-c5cccc6oc7ccccc7c56)c5ccccc45)n3)cc2)cc1]

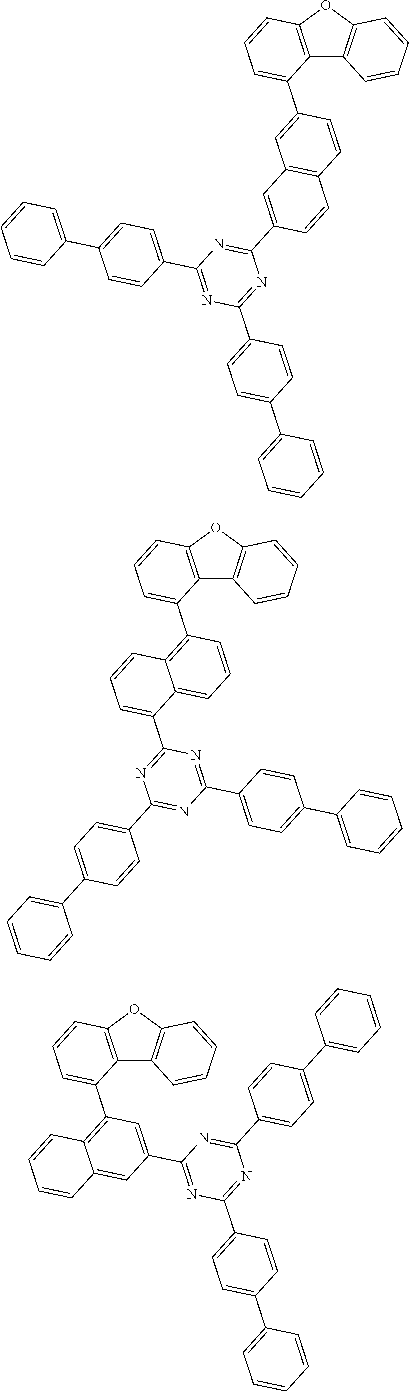 [SMILES: c1ccc(-c2ccc(-c3nc(-c4ccc(-c5ccccc5)cc4)nc(-c4ccc5ccc(-c6cccc7oc8ccccc8c67)cc5c4)n3)cc2)cc1 ; c1ccc(-c2ccc(-c3nc(-c4ccc(-c5ccccc5)cc4)nc(-c4cccc5c(-c6cccc7oc8ccccc8c67)cccc45)n3)cc2)cc1 ; c1ccc(-c2ccc(-c3nc(-c4ccc(-c5ccccc5)cc4)nc(-c4cc(-c5cccc6oc7ccccc7c56)c5ccccc5c4)n3)cc2)cc1]

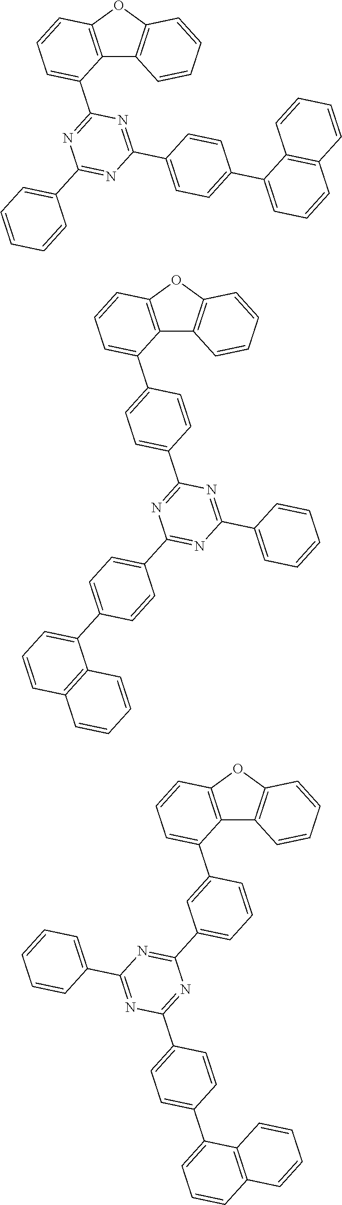 [SMILES: c1ccc(-c2nc(-c3ccc(-c4cccc5ccccc45)cc3)nc(-c3cccc4oc5ccccc5c34)n2)cc1 ; c1ccc(-c2nc(-c3ccc(-c4cccc5ccccc45)cc3)nc(-c3ccc(-c4cccc5oc6ccccc6c45)cc3)n2)cc1 ; c1ccc(-c2nc(-c3ccc(-c4cccc5ccccc45)cc3)nc(-c3cccc(-c4cccc5oc6ccccc6c45)c3)n2)cc1]

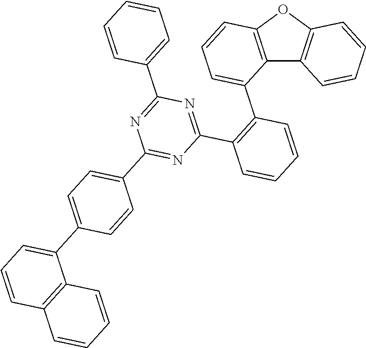 [SMILES: c1ccc(-c2nc(-c3ccc(-c4cccc5ccccc45)cc3)nc(-c3ccccc3-c3cccc4oc5ccccc5c34)n2)cc1]

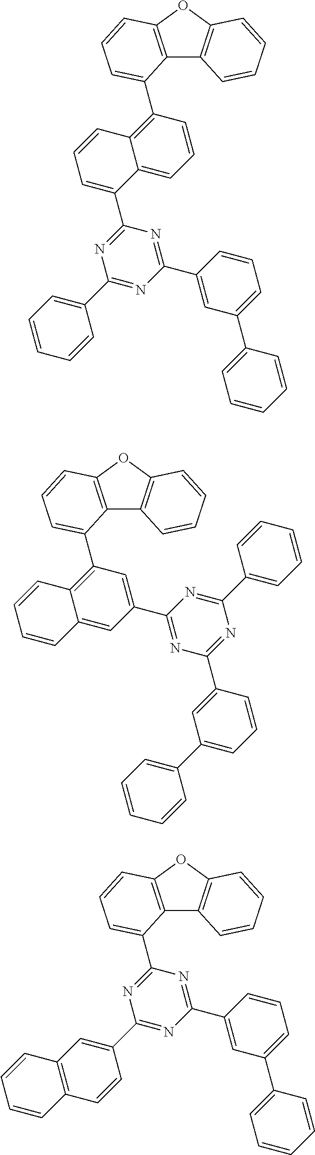

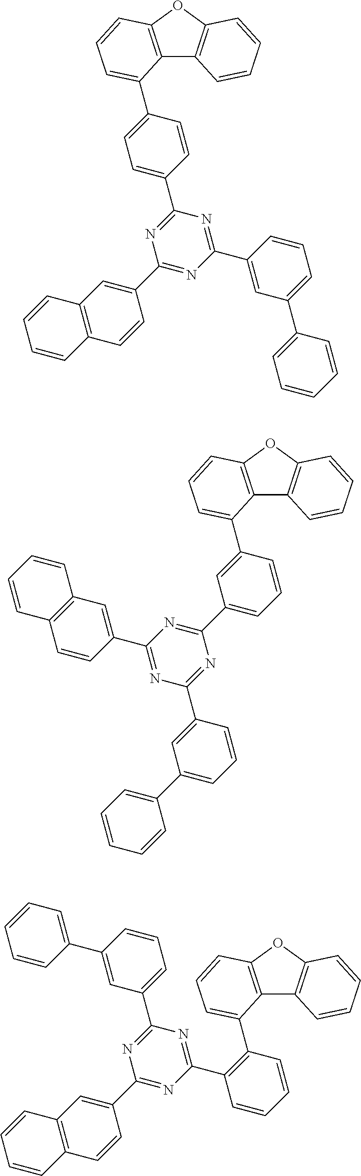 [SMILES: c1ccc(-c2cccc(-c3nc(-c4ccc(-c5cccc6oc7ccccc7c56)cc4)nc(-c4ccc5ccccc5c4)n3)c2)cc1 ; c1ccc(-c2cccc(-c3nc(-c4cccc(-c5cccc6oc7ccccc7c56)c4)nc(-c4ccc5ccccc5c4)n3)c2)cc1 ; c1ccc(-c2cccc(-c3nc(-c4ccc5ccccc5c4)nc(-c4ccccc4-c4cccc5oc6ccccc6c45)n3)c2)cc1]

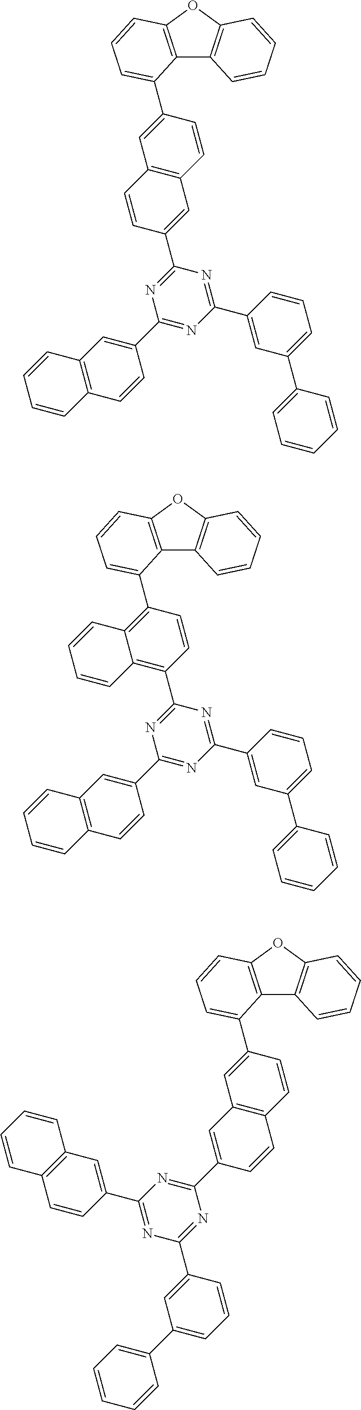 [SMILES: c1ccc(-c2cccc(-c3nc(-c4ccc5ccccc5c4)nc(-c4ccc5cc(-c6cccc7oc8ccccc8c67)ccc5c4)n3)c2)cc1 ; c1ccc(-c2cccc(-c3nc(-c4ccc5ccccc5c4)nc(-c4ccc(-c5cccc6oc7ccccc7c56)c5ccccc45)n3)c2)cc1 ; c1ccc(-c2cccc(-c3nc(-c4ccc5ccccc5c4)nc(-c4ccc5ccc(-c6cccc7oc8ccccc8c67)cc5c4)n3)c2)cc1]

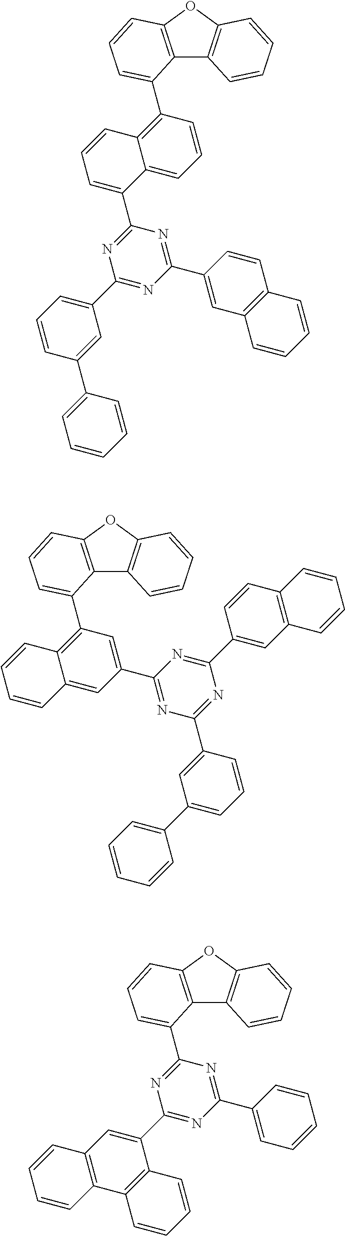 [SMILES: c1ccc(-c2cccc(-c3nc(-c4ccc5ccccc5c4)nc(-c4cccc5c(-c6cccc7oc8ccccc8c67)cccc45)n3)c2)cc1 ; c1ccc(-c2cccc(-c3nc(-c4ccc5ccccc5c4)nc(-c4cc(-c5cccc6oc7ccccc7c56)c5ccccc5c4)n3)c2)cc1 ; c1ccc(-c2nc(-c3cc4ccccc4c4ccccc34)nc(-c3cccc4oc5ccccc5c34)n2)cc1]

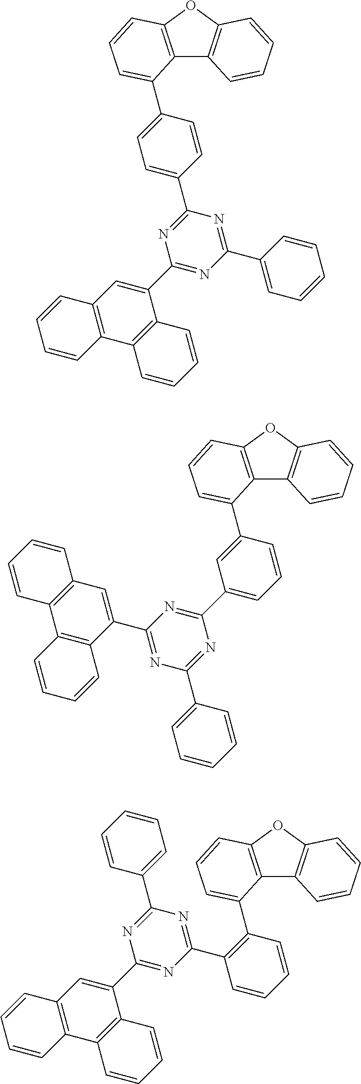 [SMILES: c1ccc(-c2nc(-c3ccc(-c4cccc5oc6ccccc6c45)cc3)nc(-c3cc4ccccc4c4ccccc34)n2)cc1 ; c1ccc(-c2nc(-c3cccc(-c4cccc5oc6ccccc6c45)c3)nc(-c3cc4ccccc4c4ccccc34)n2)cc1 ; c1ccc(-c2nc(-c3ccccc3-c3cccc4oc5ccccc5c34)nc(-c3cc4ccccc4c4ccccc34)n2)cc1]

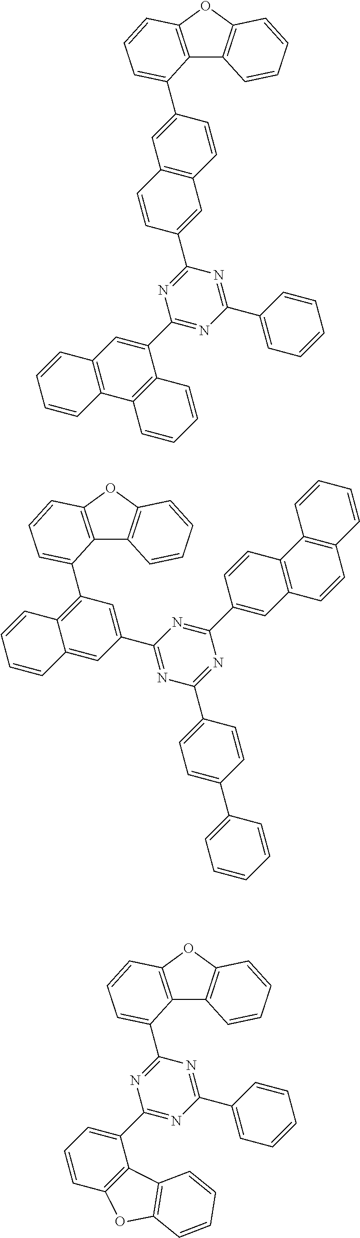 [SMILES: c1ccc(-c2nc(-c3ccc4cc(-c5cccc6oc7ccccc7c56)ccc4c3)nc(-c3cc4ccccc4c4ccccc34)n2)cc1 ; c1ccc(-c2ccc(-c3nc(-c4cc(-c5cccc6oc7ccccc7c56)c5ccccc5c4)nc(-c4ccc5c(ccc6ccccc65)c4)n3)cc2)cc1 ; c1ccc(-c2nc(-c3cccc4oc5ccccc5c34)nc(-c3cccc4oc5ccccc5c34)n2)cc1]

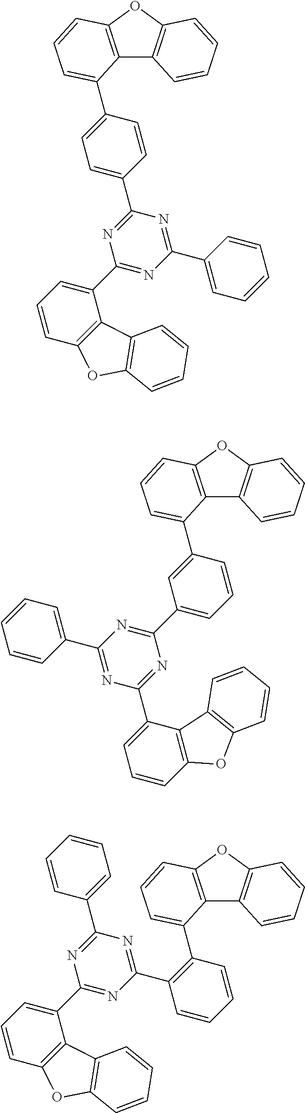 [SMILES: c1ccc(-c2nc(-c3ccc(-c4cccc5oc6ccccc6c45)cc3)nc(-c3cccc4oc5ccccc5c34)n2)cc1 ; c1ccc(-c2nc(-c3cccc(-c4cccc5oc6ccccc6c45)c3)nc(-c3cccc4oc5ccccc5c34)n2)cc1 ; c1ccc(-c2nc(-c3ccccc3-c3cccc4oc5ccccc5c34)nc(-c3cccc4oc5ccccc5c34)n2)cc1]

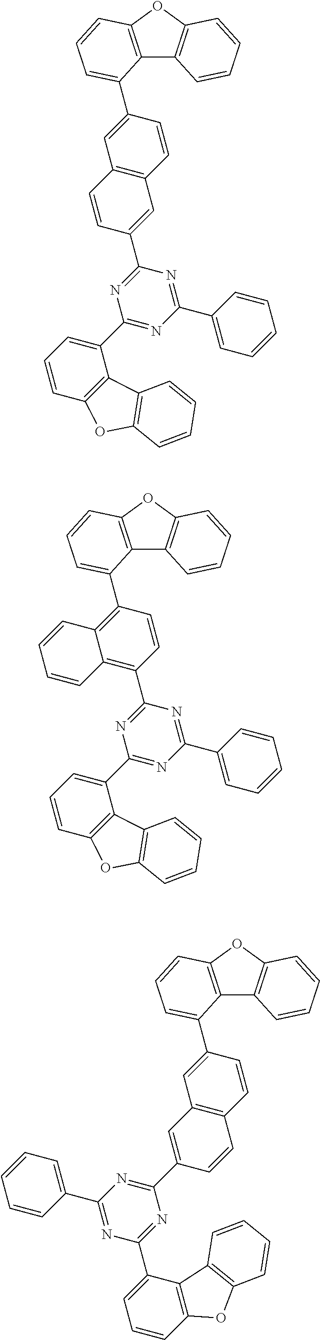 [SMILES: c1ccc(-c2nc(-c3ccc4cc(-c5cccc6oc7ccccc7c56)ccc4c3)nc(-c3cccc4oc5ccccc5c34)n2)cc1 ; c1ccc(-c2nc(-c3ccc(-c4cccc5oc6ccccc6c45)c4ccccc34)nc(-c3cccc4oc5ccccc5c34)n2)cc1 ; c1ccc(-c2nc(-c3ccc4ccc(-c5cccc6oc7ccccc7c56)cc4c3)nc(-c3cccc4oc5ccccc5c34)n2)cc1]

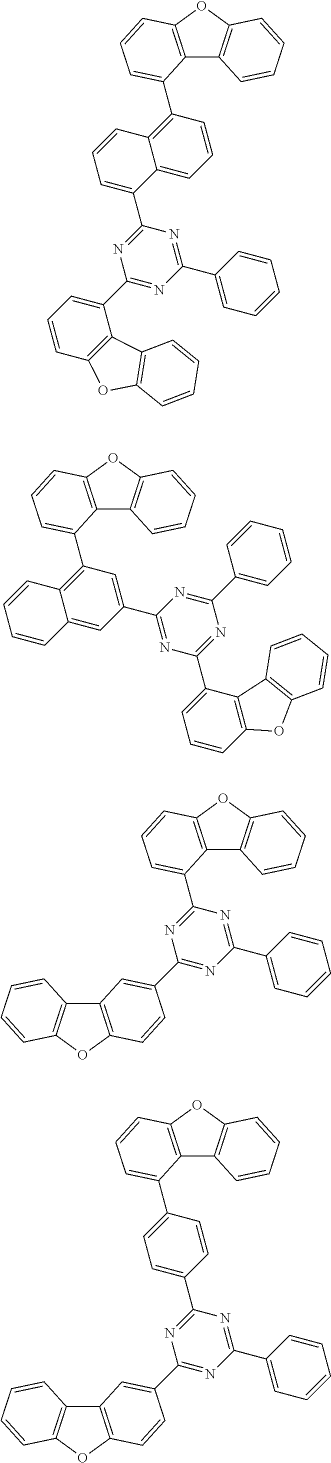

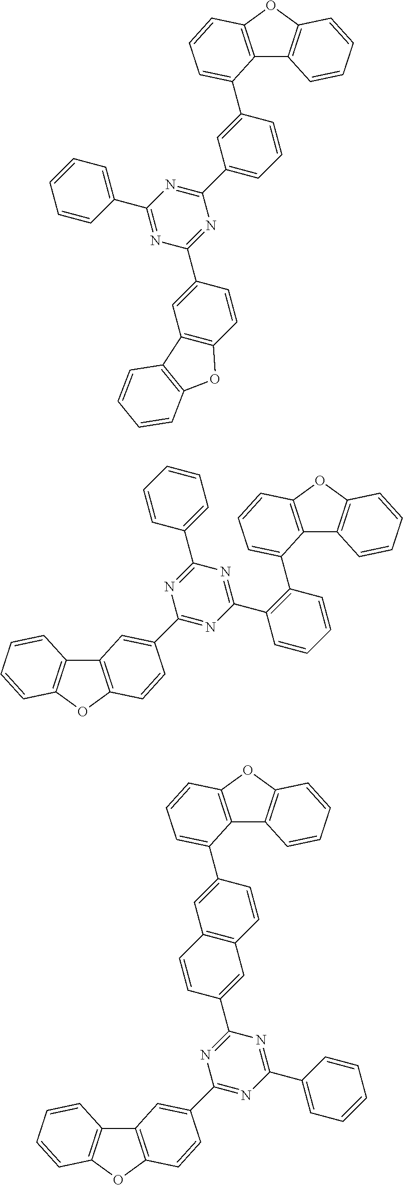

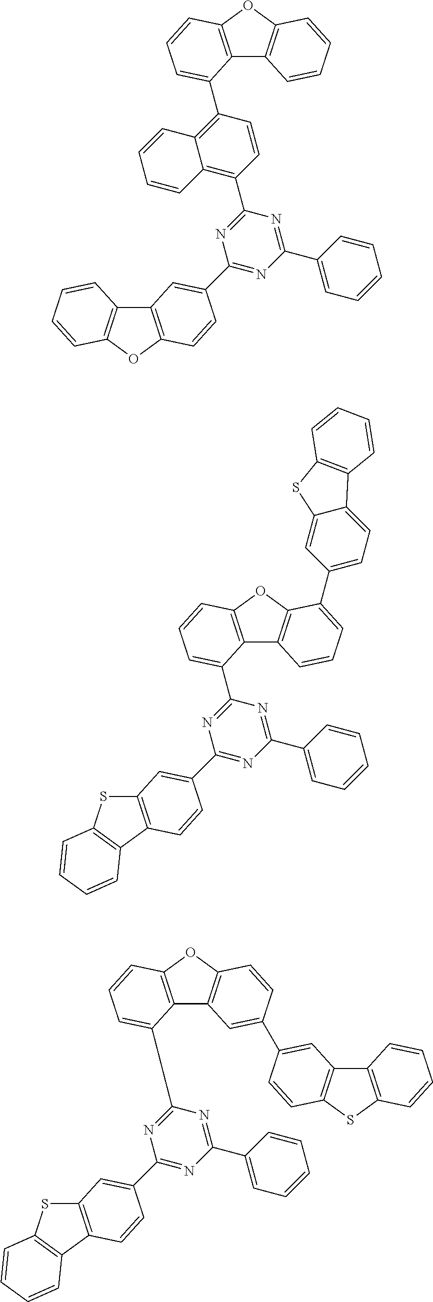 [SMILES: c1ccc(-c2nc(-c3ccc4oc5ccccc5c4c3)nc(-c3ccc(-c4cccc5oc6ccccc6c45)c4ccccc34)n2)cc1 ; c1ccc(-c2nc(-c3ccc4c(c3)sc3ccccc34)nc(-c3cccc4oc5c(-c6ccc7c(c6)sc6ccccc67)cccc5c34)n2)cc1 ; c1ccc(-c2nc(-c3ccc4c(c3)sc3ccccc34)nc(-c3cccc4oc5ccc(-c6ccc7sc8ccccc8c7c6)cc5c34)n2)cc1]

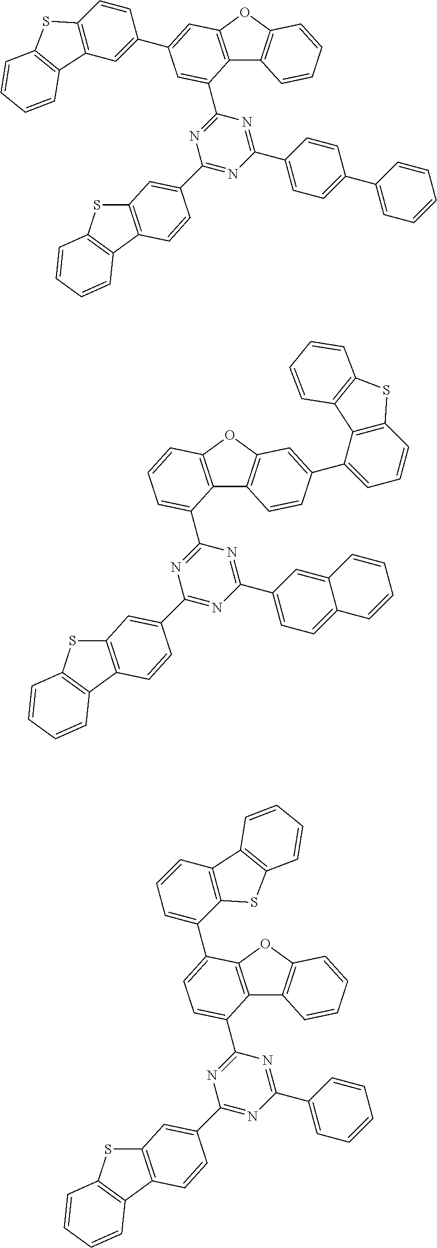 [SMILES: c1ccc(-c2ccc(-c3nc(-c4ccc5c(c4)sc4ccccc45)nc(-c4cc(-c5ccc6sc7ccccc7c6c5)cc5oc6ccccc6c45)n3)cc2)cc1 ; c1ccc2cc(-c3nc(-c4ccc5c(c4)sc4ccccc45)nc(-c4cccc5oc6cc(-c7cccc8sc9ccccc9c78)ccc6c45)n3)ccc2c1 ; c1ccc(-c2nc(-c3ccc4c(c3)sc3ccccc34)nc(-c3ccc(-c4cccc5c4sc4ccccc45)c4oc5ccccc5c34)n2)cc1]

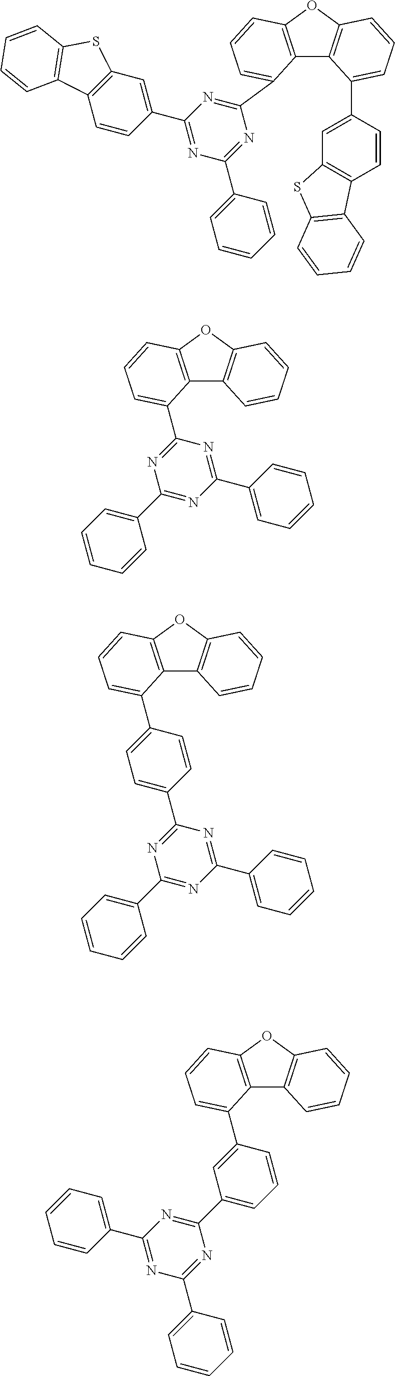 [SMILES: c1ccc(-c2nc(-c3ccc4c(c3)sc3ccccc34)nc(-c3cccc4oc5cccc(-c6ccc7c(c6)sc6ccccc67)c5c34)n2)cc1 ; c1ccc(-c2nc(-c3ccccc3)nc(-c3cccc4oc5ccccc5c34)n2)cc1 ; c1ccc(-c2nc(-c3ccccc3)nc(-c3ccc(-c4cccc5oc6ccccc6c45)cc3)n2)cc1 ; c1ccc(-c2nc(-c3ccccc3)nc(-c3cccc(-c4cccc5oc6ccccc6c45)c3)n2)cc1]

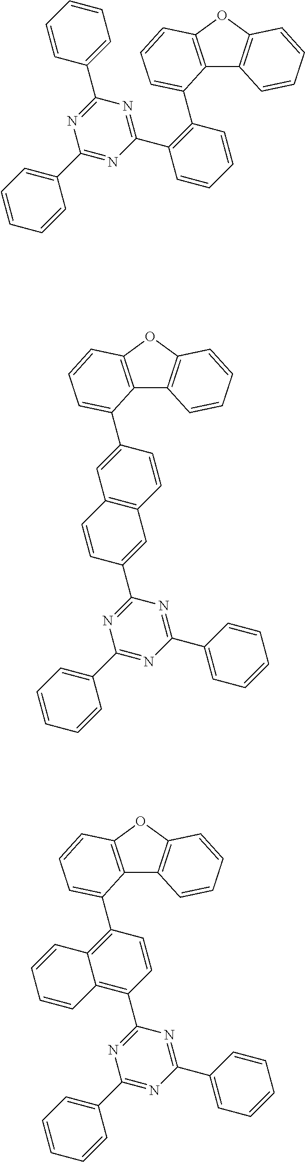 [SMILES: c1ccc(-c2nc(-c3ccccc3)nc(-c3ccccc3-c3cccc4oc5ccccc5c34)n2)cc1 ; c1ccc(-c2nc(-c3ccccc3)nc(-c3ccc4cc(-c5cccc6oc7ccccc7c56)ccc4c3)n2)cc1 ; c1ccc(-c2nc(-c3ccccc3)nc(-c3ccc(-c4cccc5oc6ccccc6c45)c4ccccc34)n2)cc1]

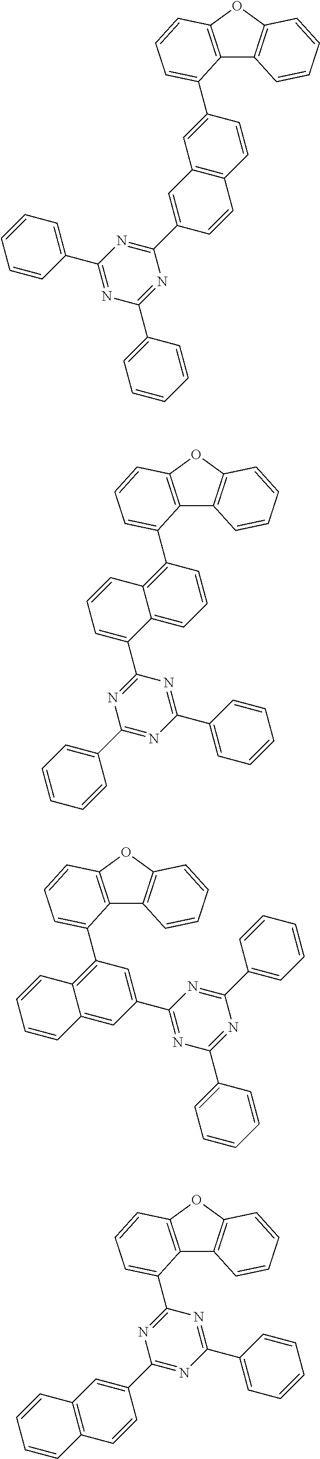 [SMILES: c1ccc(-c2nc(-c3ccccc3)nc(-c3ccc4ccc(-c5cccc6oc7ccccc7c56)cc4c3)n2)cc1 ; c1ccc(-c2nc(-c3ccccc3)nc(-c3cccc4c(-c5cccc6oc7ccccc7c56)cccc34)n2)cc1 ; c1ccc(-c2nc(-c3ccccc3)nc(-c3cc(-c4cccc5oc6ccccc6c45)c4ccccc4c3)n2)cc1 ; c1ccc(-c2nc(-c3ccc4ccccc4c3)nc(-c3cccc4oc5ccccc5c34)n2)cc1]

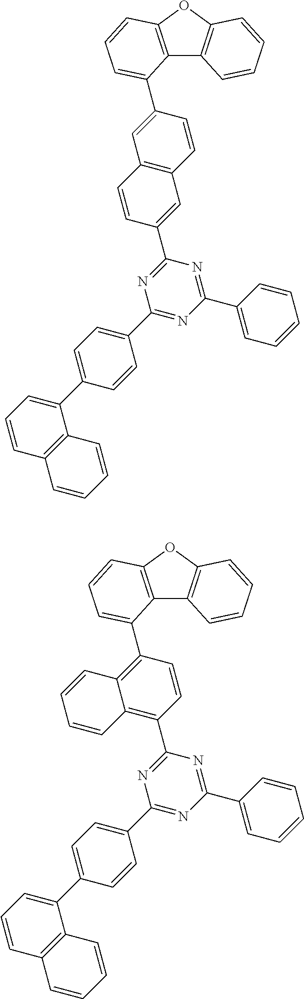 [SMILES: c1ccc(-c2nc(-c3ccc(-c4cccc5ccccc45)cc3)nc(-c3ccc4cc(-c5cccc6oc7ccccc7c56)ccc4c3)n2)cc1 ; c1ccc(-c2nc(-c3ccc(-c4cccc5ccccc45)cc3)nc(-c3ccc(-c4cccc5oc6ccccc6c45)c4ccccc34)n2)cc1]

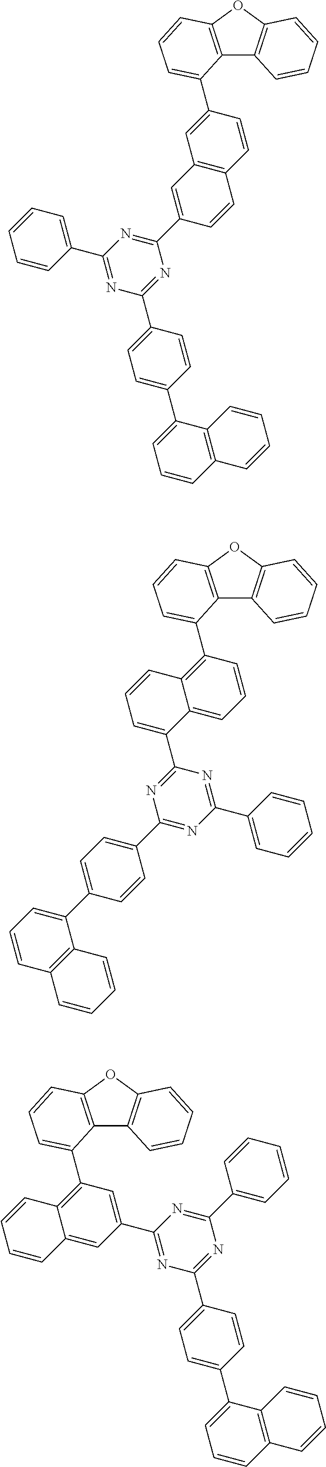 [SMILES: c1ccc(-c2nc(-c3ccc(-c4cccc5ccccc45)cc3)nc(-c3ccc4ccc(-c5cccc6oc7ccccc7c56)cc4c3)n2)cc1 ; c1ccc(-c2nc(-c3ccc(-c4cccc5ccccc45)cc3)nc(-c3cccc4c(-c5cccc6oc7ccccc7c56)cccc34)n2)cc1 ; c1ccc(-c2nc(-c3ccc(-c4cccc5ccccc45)cc3)nc(-c3cc(-c4cccc5oc6ccccc6c45)c4ccccc4c3)n2)cc1]

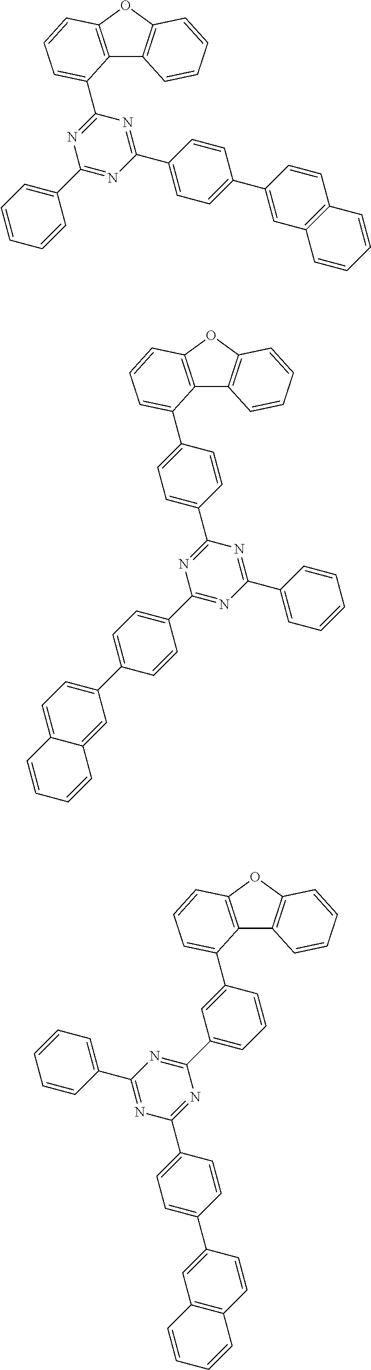 [SMILES: c1ccc(-c2nc(-c3ccc(-c4ccc5ccccc5c4)cc3)nc(-c3cccc4oc5ccccc5c34)n2)cc1 ; c1ccc(-c2nc(-c3ccc(-c4ccc5ccccc5c4)cc3)nc(-c3ccc(-c4cccc5oc6ccccc6c45)cc3)n2)cc1 ; c1ccc(-c2nc(-c3ccc(-c4ccc5ccccc5c4)cc3)nc(-c3cccc(-c4cccc5oc6ccccc6c45)c3)n2)cc1]

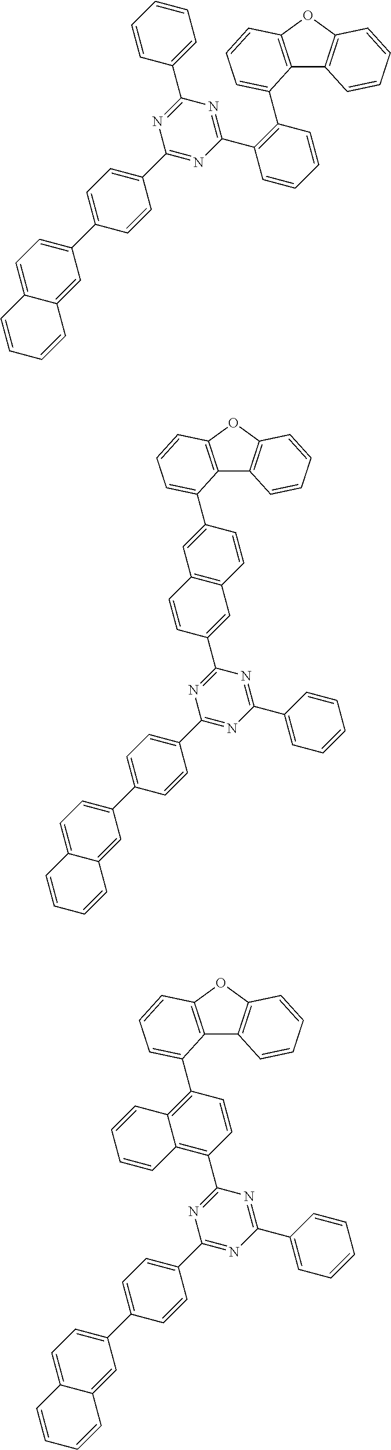 [SMILES: c1ccc(-c2nc(-c3ccc(-c4ccc5ccccc5c4)cc3)nc(-c3ccccc3-c3cccc4oc5ccccc5c34)n2)cc1 ; c1ccc(-c2nc(-c3ccc(-c4ccc5ccccc5c4)cc3)nc(-c3ccc4cc(-c5cccc6oc7ccccc7c56)ccc4c3)n2)cc1 ; c1ccc(-c2nc(-c3ccc(-c4ccc5ccccc5c4)cc3)nc(-c3ccc(-c4cccc5oc6ccccc6c45)c4ccccc34)n2)cc1]

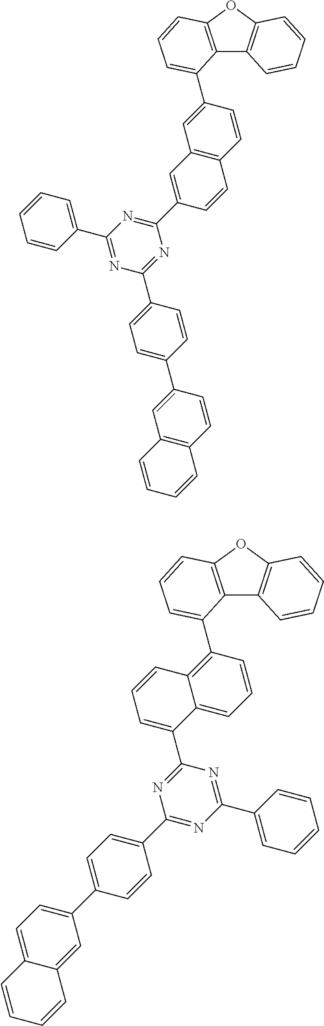 [SMILES: c1ccc(-c2nc(-c3ccc(-c4ccc5ccccc5c4)cc3)nc(-c3ccc4ccc(-c5cccc6oc7ccccc7c56)cc4c3)n2)cc1 ; c1ccc(-c2nc(-c3ccc(-c4ccc5ccccc5c4)cc3)nc(-c3cccc4c(-c5cccc6oc7ccccc7c56)cccc34)n2)cc1]

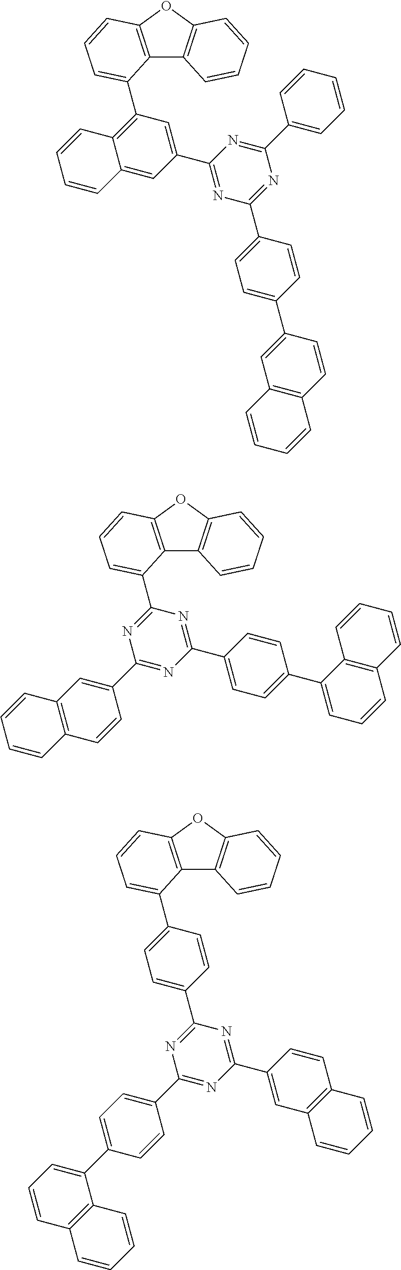 [SMILES: c1ccc(-c2nc(-c3ccc(-c4ccc5ccccc5c4)cc3)nc(-c3cc(-c4cccc5oc6ccccc6c45)c4ccccc4c3)n2)cc1 ; c1ccc2cc(-c3nc(-c4ccc(-c5cccc6ccccc56)cc4)nc(-c4cccc5oc6ccccc6c45)n3)ccc2c1 ; c1ccc2cc(-c3nc(-c4ccc(-c5cccc6ccccc56)cc4)nc(-c4ccc(-c5cccc6oc7ccccc7c56)cc4)n3)ccc2c1]

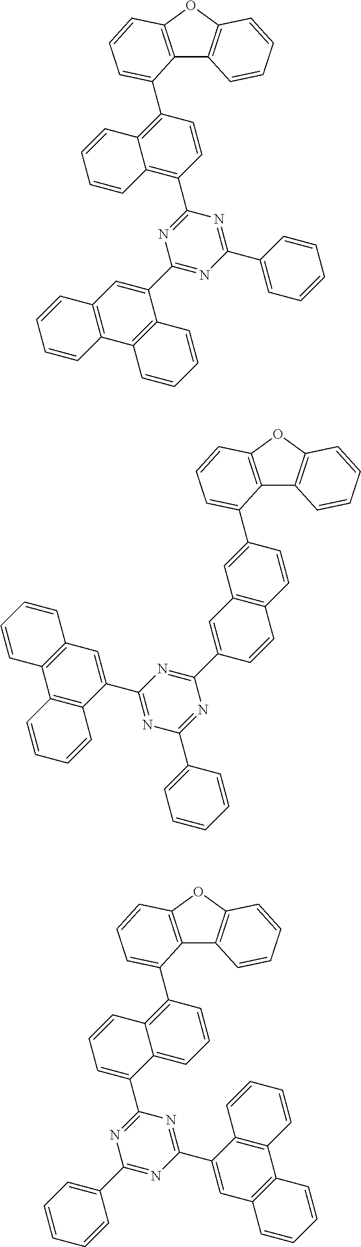 [SMILES: c1ccc(-c2nc(-c3ccc(-c4cccc5oc6ccccc6c45)c4ccccc34)nc(-c3cc4ccccc4c4ccccc34)n2)cc1 ; c1ccc(-c2nc(-c3ccc4ccc(-c5cccc6oc7ccccc7c56)cc4c3)nc(-c3cc4ccccc4c4ccccc34)n2)cc1 ; c1ccc(-c2nc(-c3cccc4c(-c5cccc6oc7ccccc7c56)cccc34)nc(-c3cc4ccccc4c4ccccc34)n2)cc1]

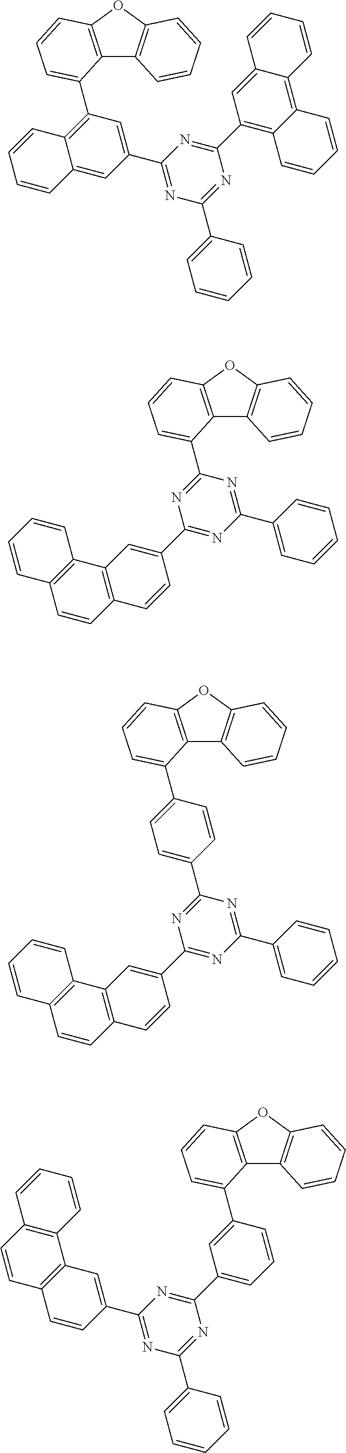 [SMILES: c1ccc(-c2nc(-c3cc(-c4cccc5oc6ccccc6c45)c4ccccc4c3)nc(-c3cc4ccccc4c4ccccc34)n2)cc1 ; c1ccc(-c2nc(-c3ccc4ccc5ccccc5c4c3)nc(-c3cccc4oc5ccccc5c34)n2)cc1 ; c1ccc(-c2nc(-c3ccc(-c4cccc5oc6ccccc6c45)cc3)nc(-c3ccc4ccc5ccccc5c4c3)n2)cc1 ; c1ccc(-c2nc(-c3cccc(-c4cccc5oc6ccccc6c45)c3)nc(-c3ccc4ccc5ccccc5c4c3)n2)cc1]

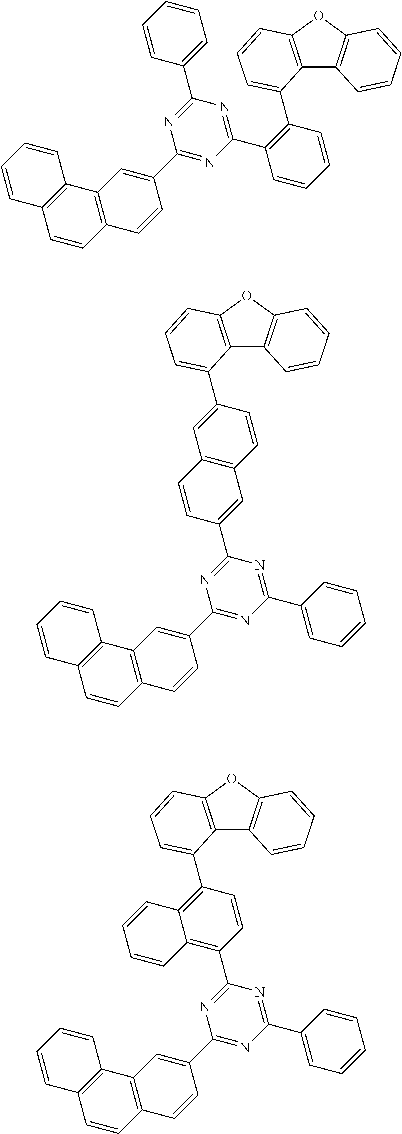 [SMILES: c1ccc(-c2nc(-c3ccc4ccc5ccccc5c4c3)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4ccc5ccccc5c4c3)n2)cc1 ; c1ccc(-c2nc(-c3ccc4ccc5ccccc5c4c3)nc(-c3ccc(-c4cccc5oc6ccccc6c45)c4ccccc34)n2)cc1]

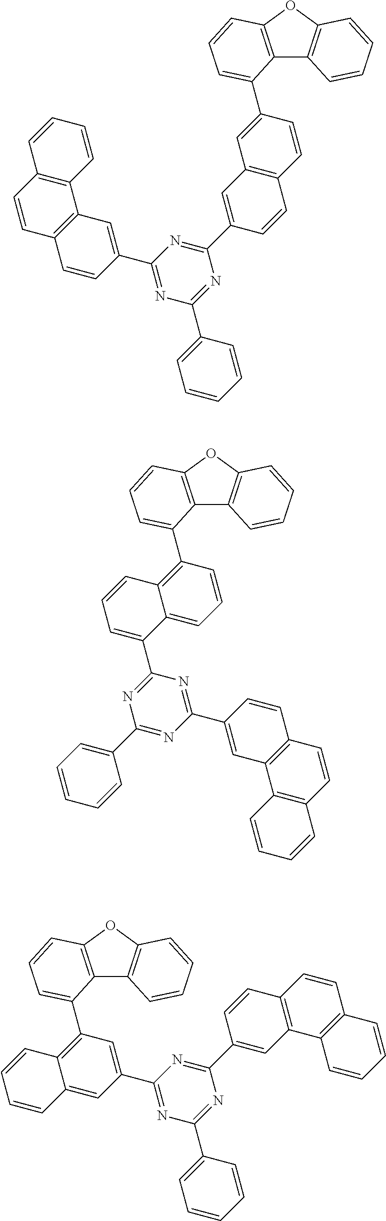 [SMILES: c1ccc(-c2nc(-c3ccc4ccc(-c5cccc6oc7ccccc7c56)cc4c3)nc(-c3ccc4ccc5ccccc5c4c3)n2)cc1 ; c1ccc(-c2nc(-c3ccc4ccc5ccccc5c4c3)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4ccc5ccccc5c4c3)n2)cc1]

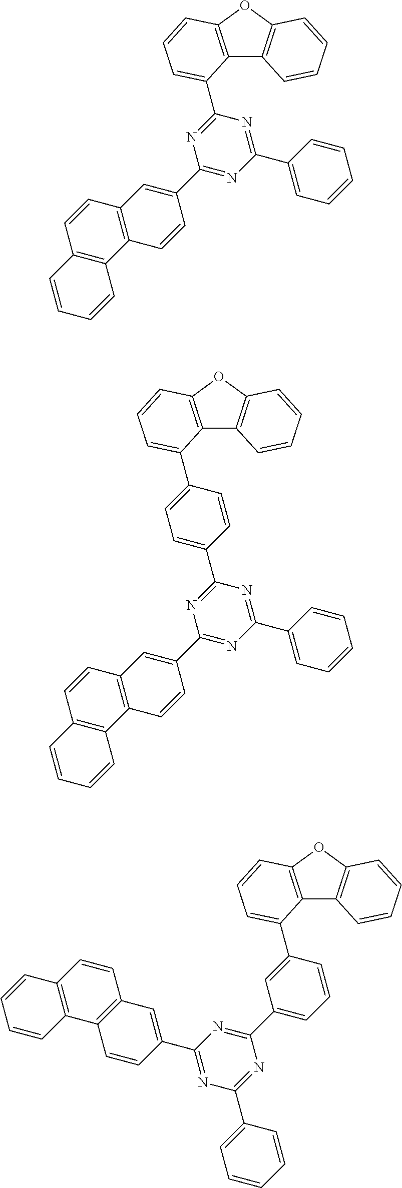 [SMILES: c1ccc(-c2nc(-c3ccc4c(ccc5ccccc54)c3)nc(-c3cccc4oc5ccccc5c34)n2)cc1 ; c1ccc(-c2nc(-c3ccc(-c4cccc5oc6ccccc6c45)cc3)nc(-c3ccc4c(ccc5ccccc54)c3)n2)cc1 ; c1ccc(-c2nc(-c3cccc(-c4cccc5oc6ccccc6c45)c3)nc(-c3ccc4c(ccc5ccccc54)c3)n2)cc1]

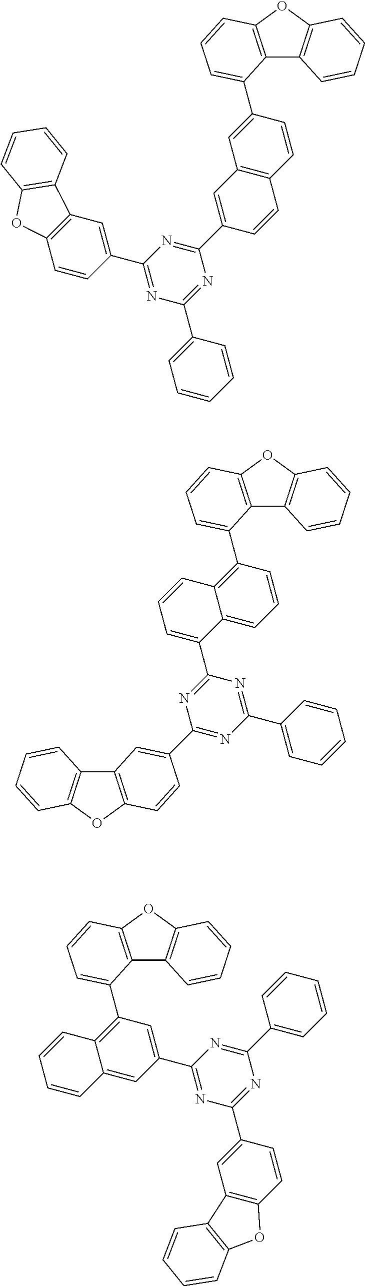 [SMILES: c1ccc(-c2nc(-c3ccc4ccc(-c5cccc6oc7ccccc7c56)cc4c3)nc(-c3ccc4oc5ccccc5c4c3)n2)cc1 ; c1ccc(-c2nc(-c3ccc4oc5ccccc5c4c3)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4oc5ccccc5c4c3)n2)cc1]

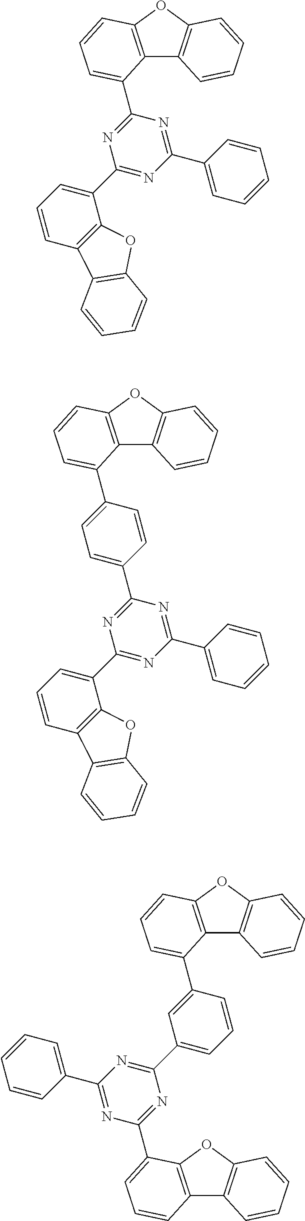 [SMILES: c1ccc(-c2nc(-c3cccc4c3oc3ccccc34)nc(-c3cccc4oc5ccccc5c34)n2)cc1 ; c1ccc(-c2nc(-c3ccc(-c4cccc5oc6ccccc6c45)cc3)nc(-c3cccc4c3oc3ccccc34)n2)cc1 ; c1ccc(-c2nc(-c3cccc(-c4cccc5oc6ccccc6c45)c3)nc(-c3cccc4c3oc3ccccc34)n2)cc1]

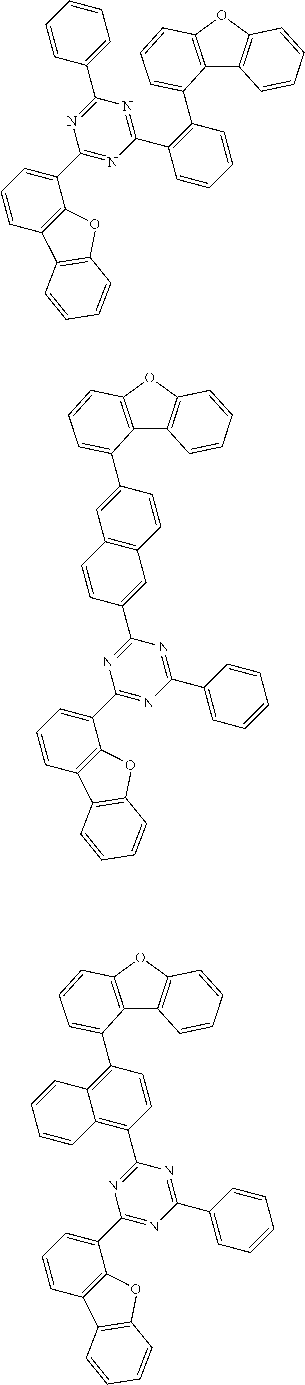 [SMILES: c1ccc(-c2nc(-c3ccccc3-c3cccc4oc5ccccc5c34)nc(-c3cccc4c3oc3ccccc34)n2)cc1 ; c1ccc(-c2nc(-c3ccc4cc(-c5cccc6oc7ccccc7c56)ccc4c3)nc(-c3cccc4c3oc3ccccc34)n2)cc1 ; c1ccc(-c2nc(-c3ccc(-c4cccc5oc6ccccc6c45)c4ccccc34)nc(-c3cccc4c3oc3ccccc34)n2)cc1]

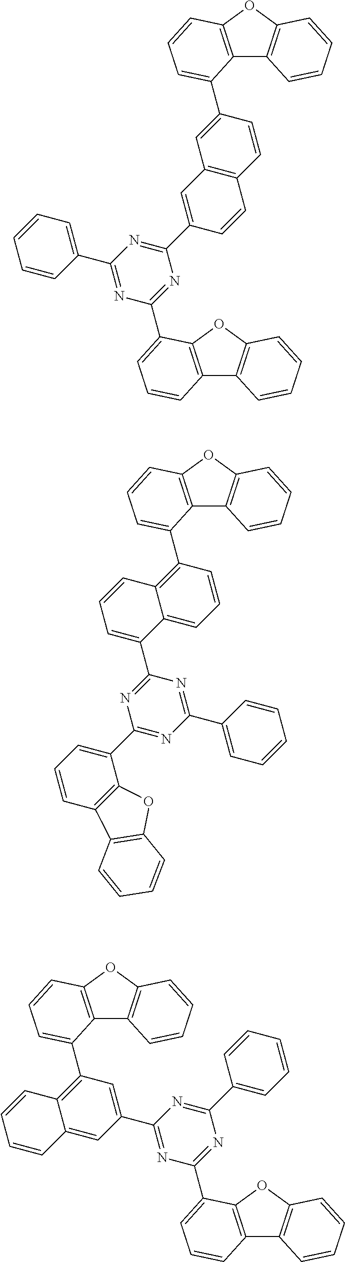 [SMILES: c1ccc(-c2nc(-c3ccc4ccc(-c5cccc6oc7ccccc7c56)cc4c3)nc(-c3cccc4c3oc3ccccc34)n2)cc1 ; c1ccc(-c2nc(-c3cccc4c(-c5cccc6oc7ccccc7c56)cccc34)nc(-c3cccc4c3oc3ccccc34)n2)cc1 ; c1ccc(-c2nc(-c3cc(-c4cccc5oc6ccccc6c45)c4ccccc4c3)nc(-c3cccc4c3oc3ccccc34)n2)cc1]

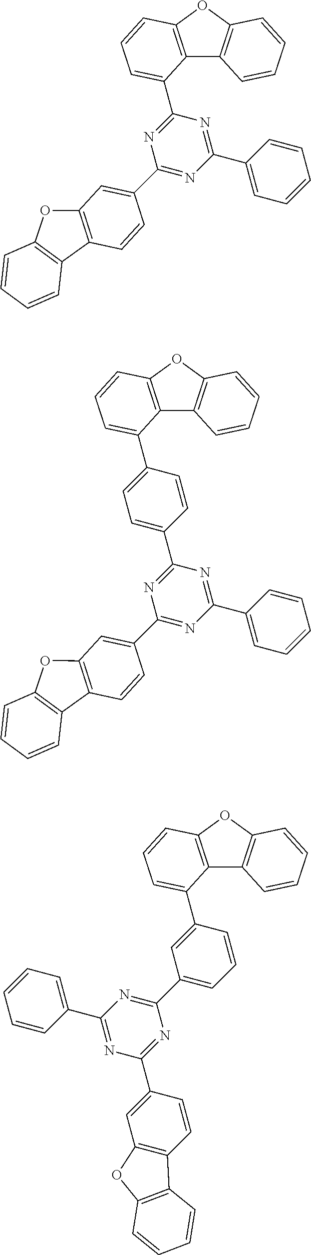 [SMILES: c1ccc(-c2nc(-c3ccc4c(c3)oc3ccccc34)nc(-c3cccc4oc5ccccc5c34)n2)cc1 ; c1ccc(-c2nc(-c3ccc(-c4cccc5oc6ccccc6c45)cc3)nc(-c3ccc4c(c3)oc3ccccc34)n2)cc1 ; c1ccc(-c2nc(-c3cccc(-c4cccc5oc6ccccc6c45)c3)nc(-c3ccc4c(c3)oc3ccccc34)n2)cc1]

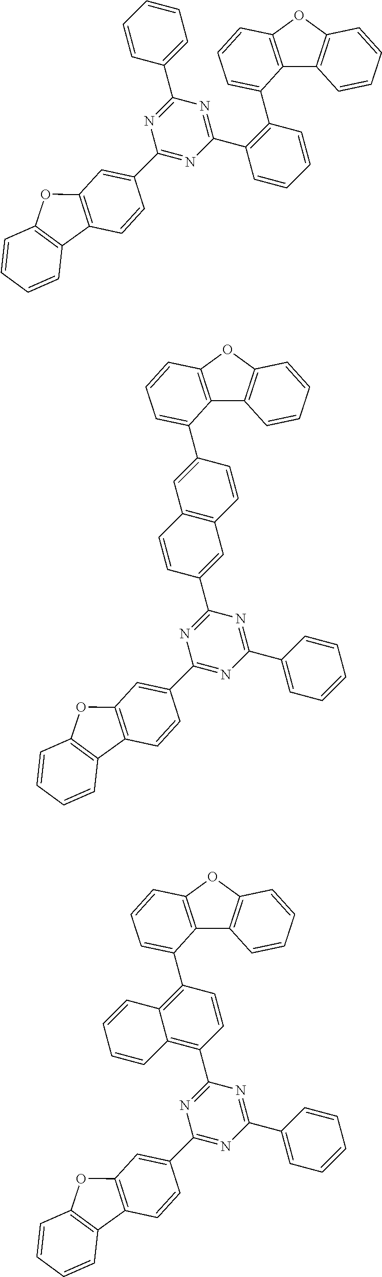 [SMILES: c1ccc(-c2nc(-c3ccc4c(c3)oc3ccccc34)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4c(c3)oc3ccccc34)n2)cc1 ; c1ccc(-c2nc(-c3ccc4c(c3)oc3ccccc34)nc(-c3ccc(-c4cccc5oc6ccccc6c45)c4ccccc34)n2)cc1]

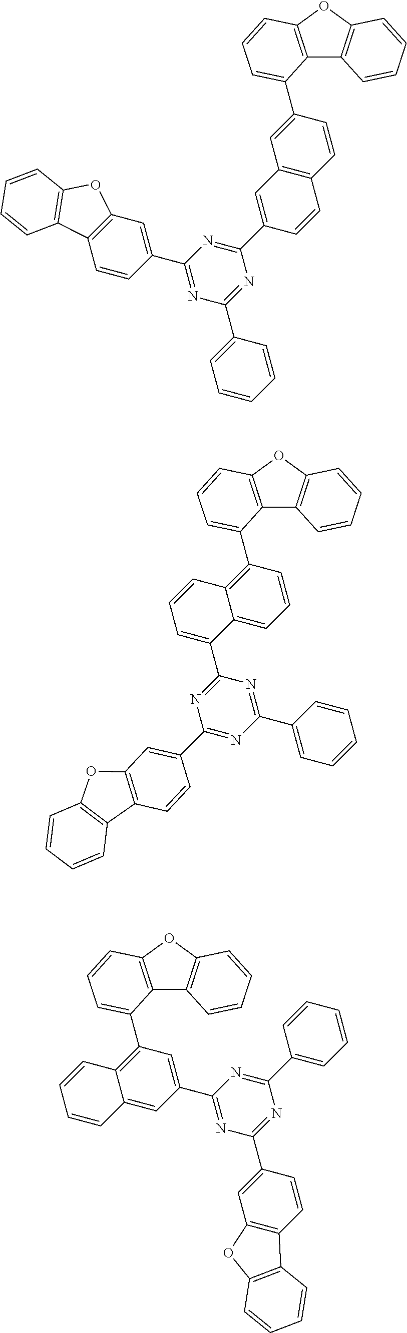 [SMILES: c1ccc(-c2nc(-c3ccc4ccc(-c5cccc6oc7ccccc7c56)cc4c3)nc(-c3ccc4c(c3)oc3ccccc34)n2)cc1 ; c1ccc(-c2nc(-c3ccc4c(c3)oc3ccccc34)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4c(c3)oc3ccccc34)n2)cc1]

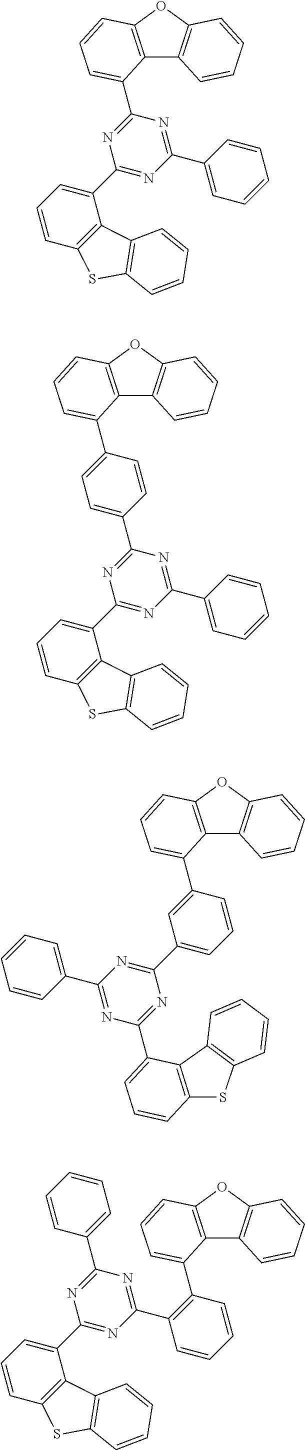 [SMILES: c1ccc(-c2nc(-c3cccc4oc5ccccc5c34)nc(-c3cccc4sc5ccccc5c34)n2)cc1 ; c1ccc(-c2nc(-c3ccc(-c4cccc5oc6ccccc6c45)cc3)nc(-c3cccc4sc5ccccc5c34)n2)cc1 ; c1ccc(-c2nc(-c3cccc(-c4cccc5oc6ccccc6c45)c3)nc(-c3cccc4sc5ccccc5c34)n2)cc1 ; c1ccc(-c2nc(-c3ccccc3-c3cccc4oc5ccccc5c34)nc(-c3cccc4sc5ccccc5c34)n2)cc1]

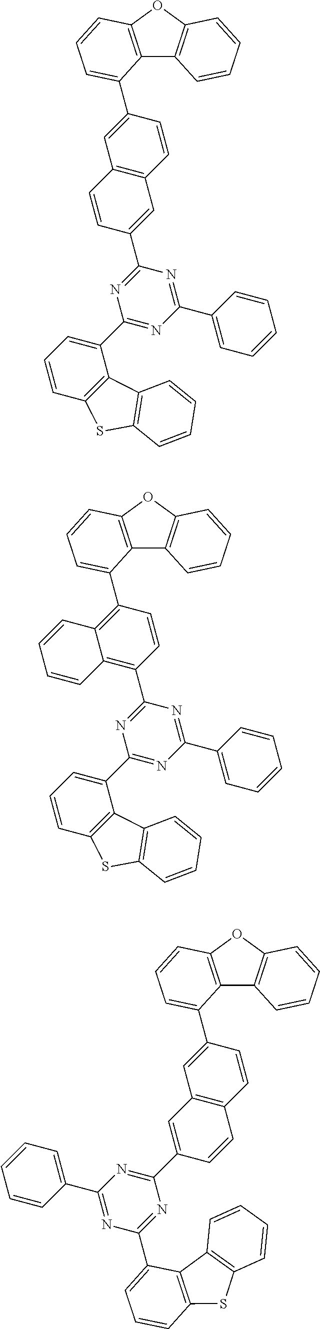 [SMILES: c1ccc(-c2nc(-c3ccc4cc(-c5cccc6oc7ccccc7c56)ccc4c3)nc(-c3cccc4sc5ccccc5c34)n2)cc1 ; c1ccc(-c2nc(-c3ccc(-c4cccc5oc6ccccc6c45)c4ccccc34)nc(-c3cccc4sc5ccccc5c34)n2)cc1 ; c1ccc(-c2nc(-c3ccc4ccc(-c5cccc6oc7ccccc7c56)cc4c3)nc(-c3cccc4sc5ccccc5c34)n2)cc1]

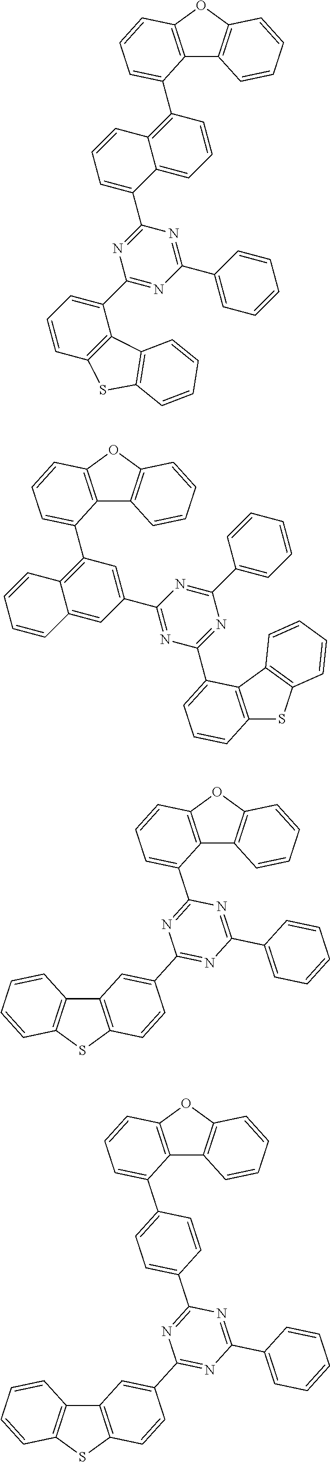 [SMILES: c1ccc(-c2nc(-c3cccc4c(-c5cccc6oc7ccccc7c56)cccc34)nc(-c3cccc4sc5ccccc5c34)n2)cc1 ; c1ccc(-c2nc(-c3cc(-c4cccc5oc6ccccc6c45)c4ccccc4c3)nc(-c3cccc4sc5ccccc5c34)n2)cc1 ; c1ccc(-c2nc(-c3ccc4sc5ccccc5c4c3)nc(-c3cccc4oc5ccccc5c34)n2)cc1 ; c1ccc(-c2nc(-c3ccc(-c4cccc5oc6ccccc6c45)cc3)nc(-c3ccc4sc5ccccc5c4c3)n2)cc1]

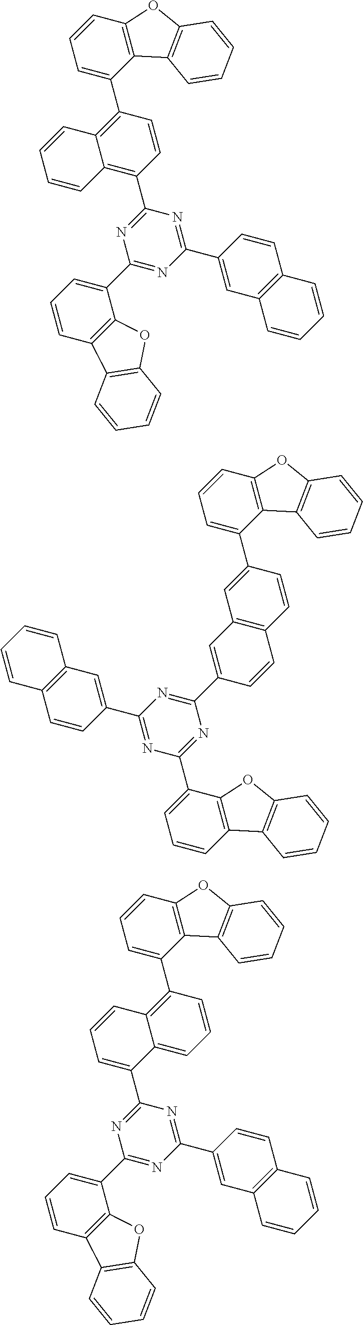

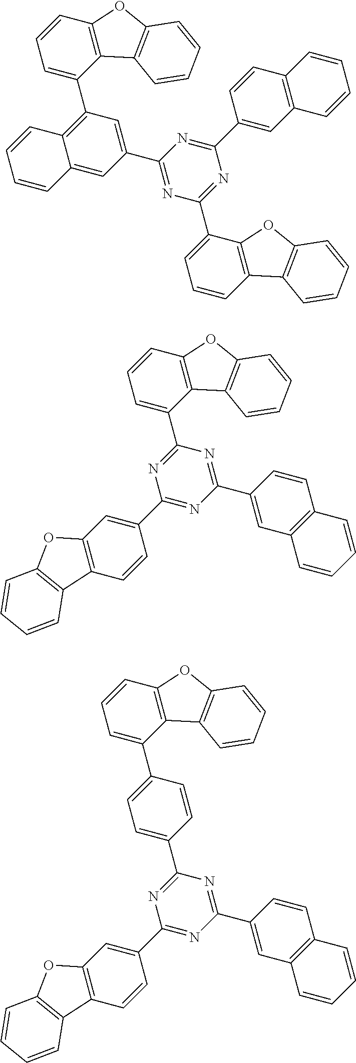

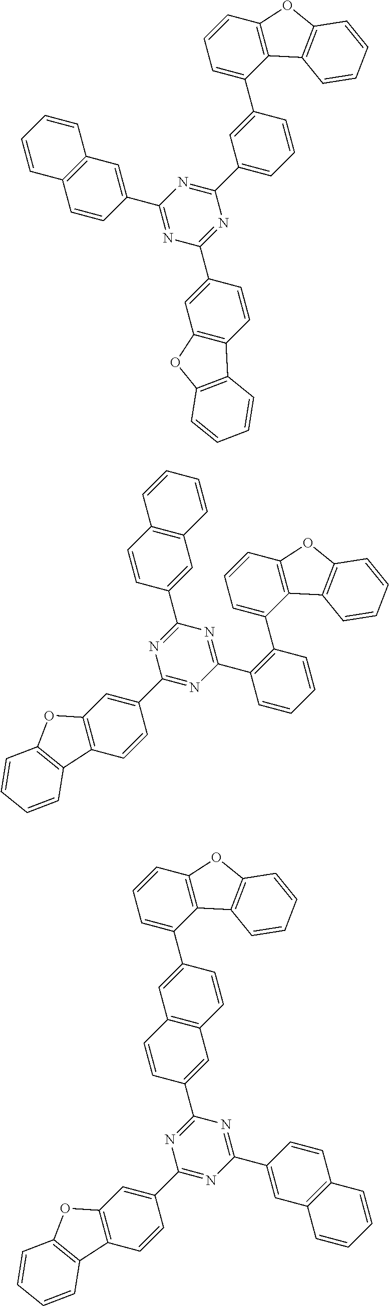

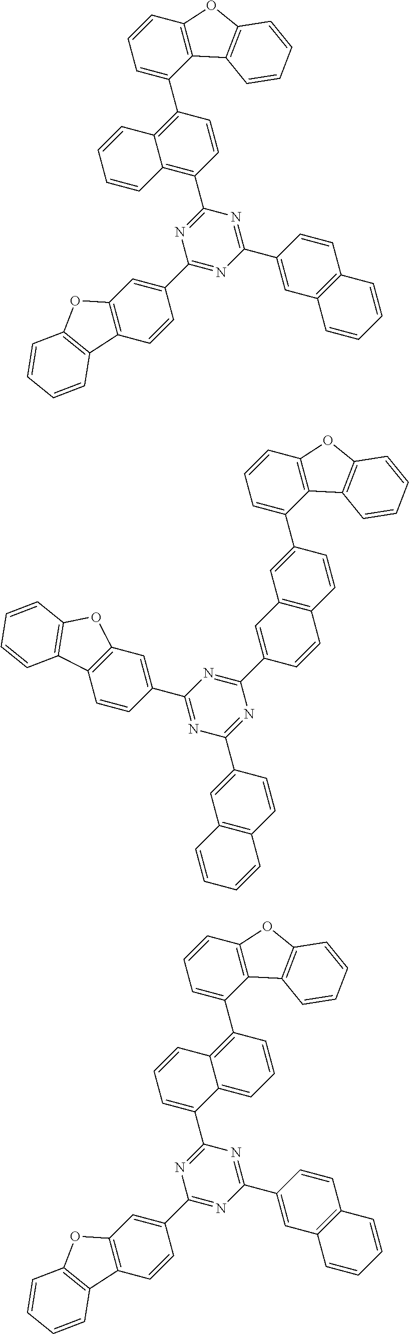 [SMILES: c1ccc2cc(-c3nc(-c4ccc5c(c4)oc4ccccc45)nc(-c4ccc(-c5cccc6oc7ccccc7c56)c5ccccc45)n3)ccc2c1 ; c1ccc2cc(-c3nc(-c4ccc5ccc(-c6cccc7oc8ccccc8c67)cc5c4)nc(-c4ccc5c(c4)oc4ccccc45)n3)ccc2c1 ; c1ccc2cc(-c3nc(-c4ccc5c(c4)oc4ccccc45)nc(-c4cccc5c(-c6cccc7oc8ccccc8c67)cccc45)n3)ccc2c1]

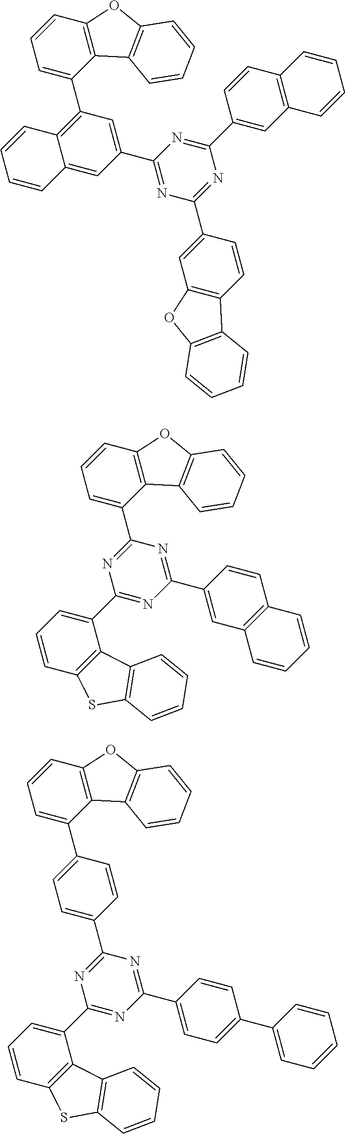 [SMILES: c1ccc2cc(-c3nc(-c4cc(-c5cccc6oc7ccccc7c56)c5ccccc5c4)nc(-c4ccc5c(c4)oc4ccccc45)n3)ccc2c1 ; c1ccc2cc(-c3nc(-c4cccc5oc6ccccc6c45)nc(-c4cccc5sc6ccccc6c45)n3)ccc2c1 ; c1ccc(-c2ccc(-c3nc(-c4ccc(-c5cccc6oc7ccccc7c56)cc4)nc(-c4cccc5sc6ccccc6c45)n3)cc2)cc1]

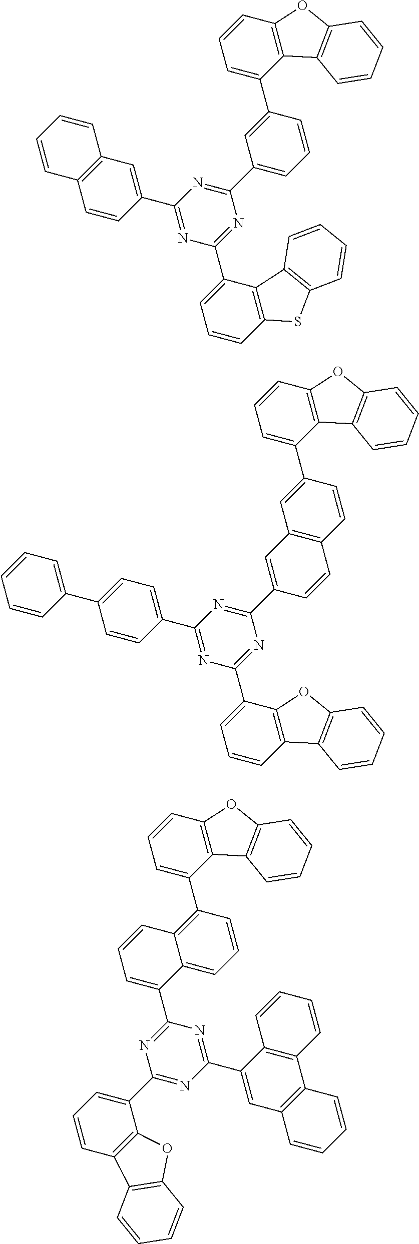 [SMILES: c1cc(-c2nc(-c3ccc4ccccc4c3)nc(-c3cccc4sc5ccccc5c34)n2)cc(-c2cccc3oc4ccccc4c23)c1 ; c1ccc(-c2ccc(-c3nc(-c4ccc5ccc(-c6cccc7oc8ccccc8c67)cc5c4)nc(-c4cccc5c4oc4ccccc45)n3)cc2)cc1 ; c1ccc2c(c1)cc(-c1nc(-c3cccc4c(-c5cccc6oc7ccccc7c56)cccc34)nc(-c3cccc4c3oc3ccccc34)n1)c1ccccc12]

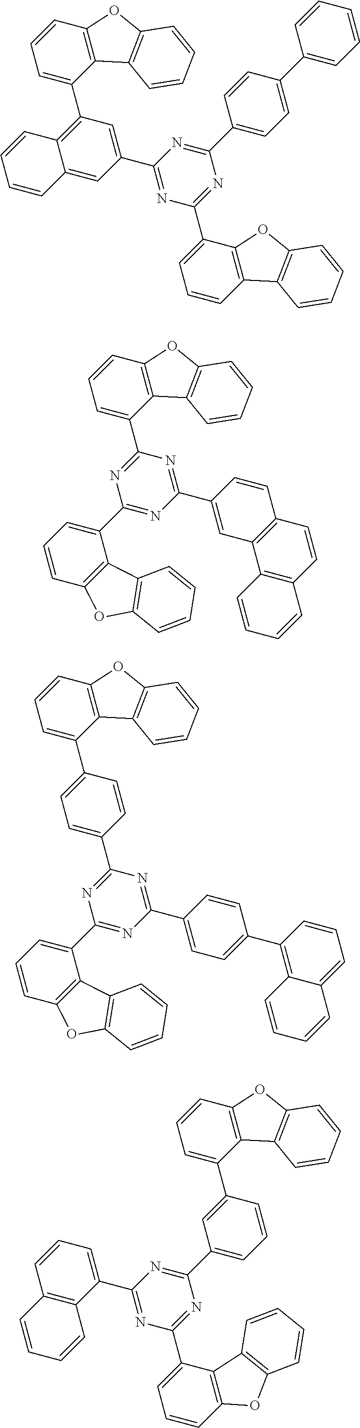 [SMILES: c1ccc(-c2ccc(-c3nc(-c4cc(-c5cccc6oc7ccccc7c56)c5ccccc5c4)nc(-c4cccc5c4oc4ccccc45)n3)cc2)cc1 ; c1ccc2c(c1)ccc1ccc(-c3nc(-c4cccc5oc6ccccc6c45)nc(-c4cccc5oc6ccccc6c45)n3)cc12 ; c1ccc2c(-c3ccc(-c4nc(-c5ccc(-c6cccc7oc8ccccc8c67)cc5)nc(-c5cccc6oc7ccccc7c56)n4)cc3)cccc2c1 ; c1cc(-c2nc(-c3cccc4ccccc34)nc(-c3cccc4oc5ccccc5c34)n2)cc(-c2cccc3oc4ccccc4c23)c1]

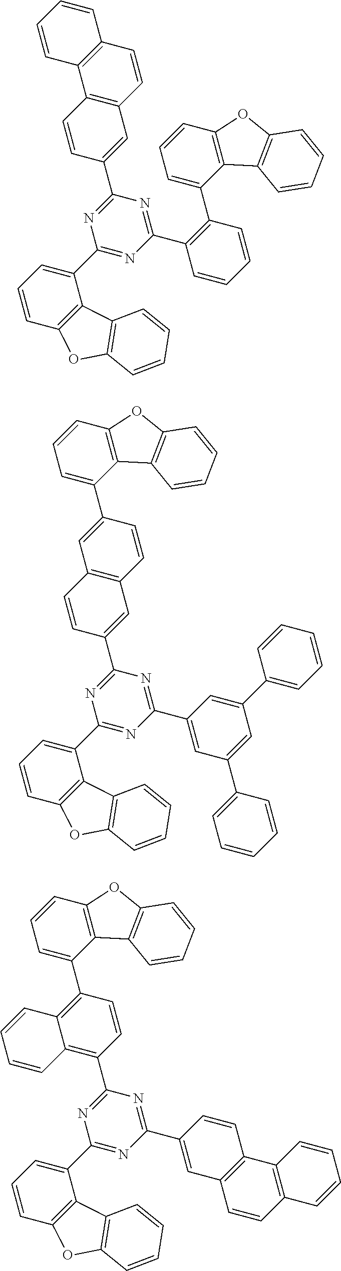

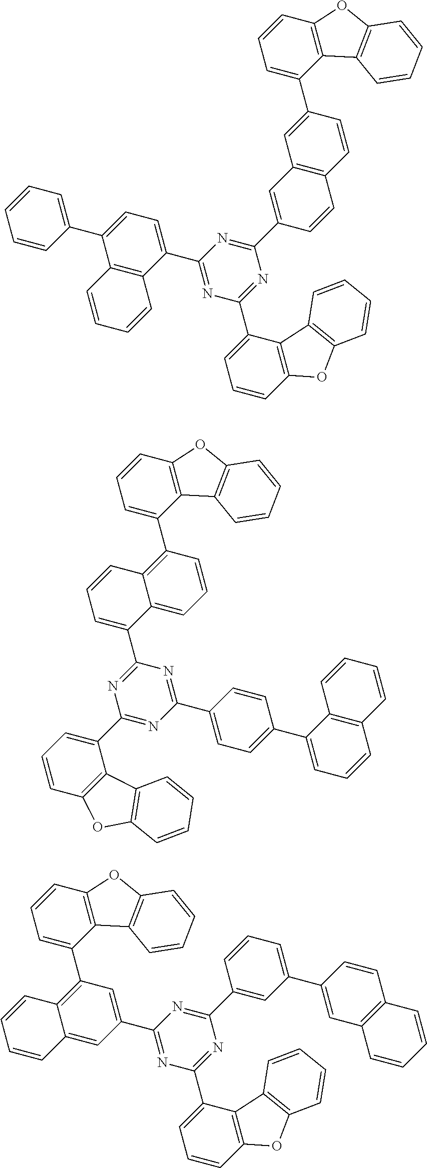 [SMILES: c1ccc(-c2ccc(-c3nc(-c4ccc5ccc(-c6cccc7oc8ccccc8c67)cc5c4)nc(-c4cccc5oc6ccccc6c45)n3)c3ccccc23)cc1 ; c1ccc2c(-c3ccc(-c4nc(-c5cccc6c(-c7cccc8oc9ccccc9c78)cccc56)nc(-c5cccc6oc7ccccc7c56)n4)cc3)cccc2c1 ; c1cc(-c2ccc3ccccc3c2)cc(-c2nc(-c3cc(-c4cccc5oc6ccccc6c45)c4ccccc4c3)nc(-c3cccc4oc5ccccc5c34)n2)c1]

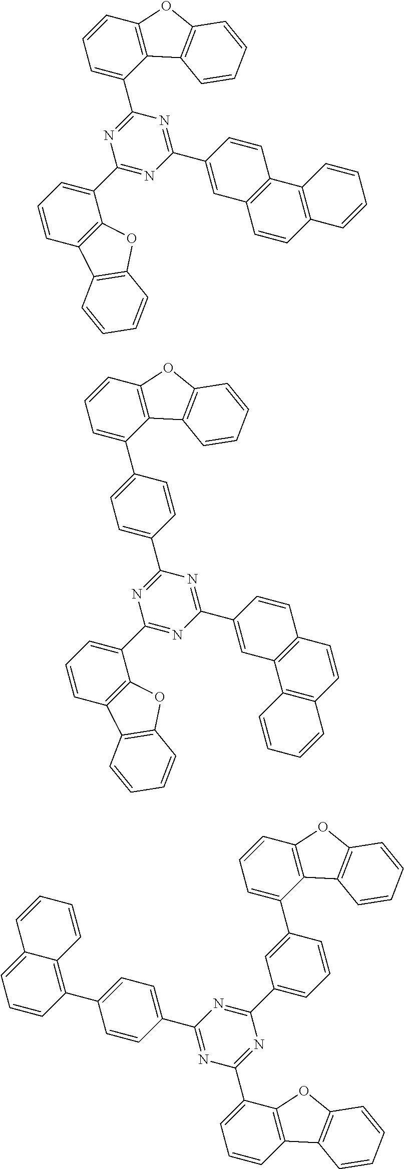

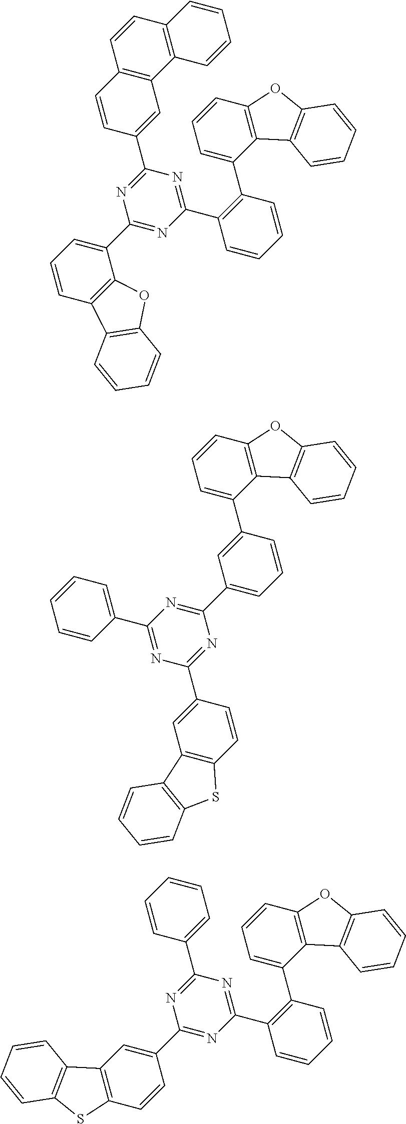

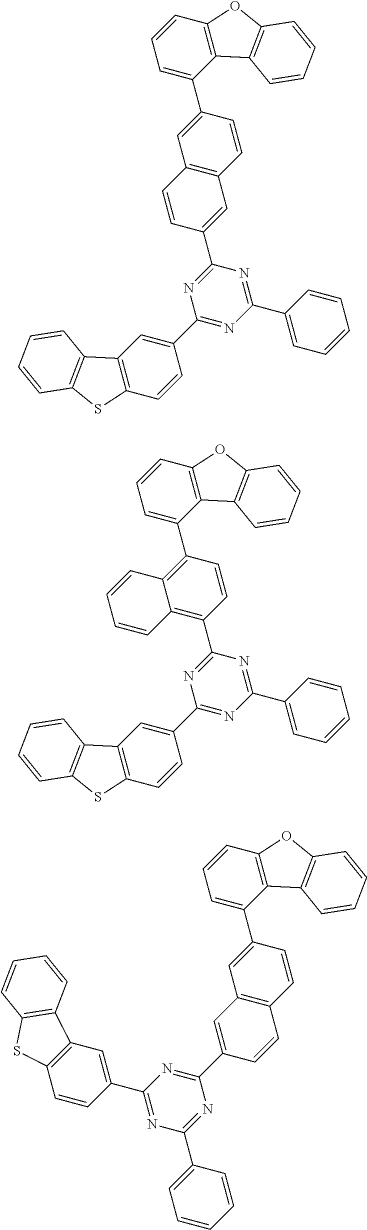 [SMILES: c1ccc(-c2nc(-c3ccc4cc(-c5cccc6oc7ccccc7c56)ccc4c3)nc(-c3ccc4sc5ccccc5c4c3)n2)cc1 ; c1ccc(-c2nc(-c3ccc4sc5ccccc5c4c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4sc5ccccc5c4c3)n2)cc1]

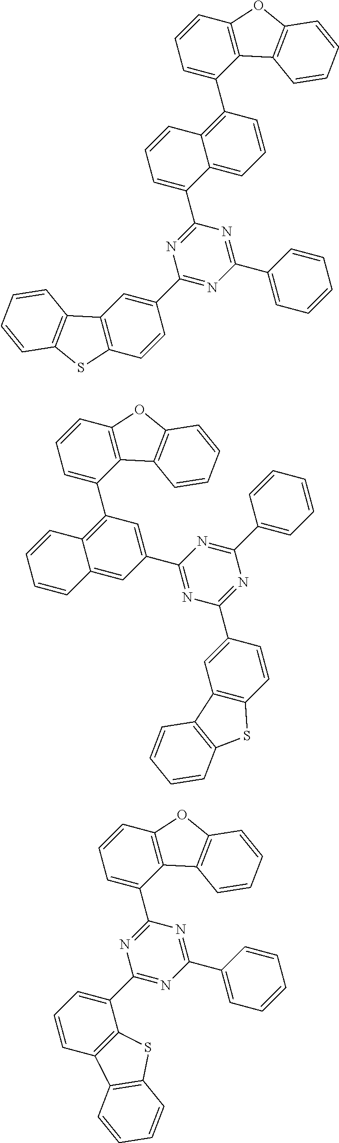

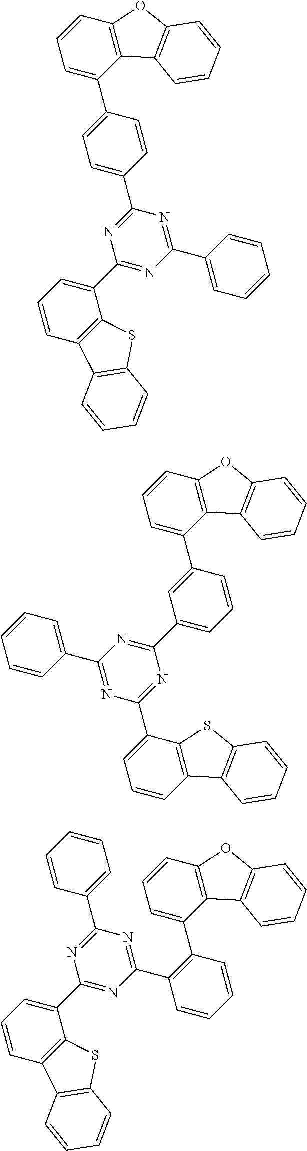 [SMILES: c1ccc(-c2nc(-c3ccc(-c4cccc5oc6ccccc6c45)cc3)nc(-c3cccc4c3sc3ccccc34)n2)cc1 ; c1ccc(-c2nc(-c3cccc(-c4cccc5oc6ccccc6c45)c3)nc(-c3cccc4c3sc3ccccc34)n2)cc1 ; c1ccc(-c2nc(-c3ccccc3-c3cccc4oc5ccccc5c34)nc(-c3cccc4c3sc3ccccc34)n2)cc1]

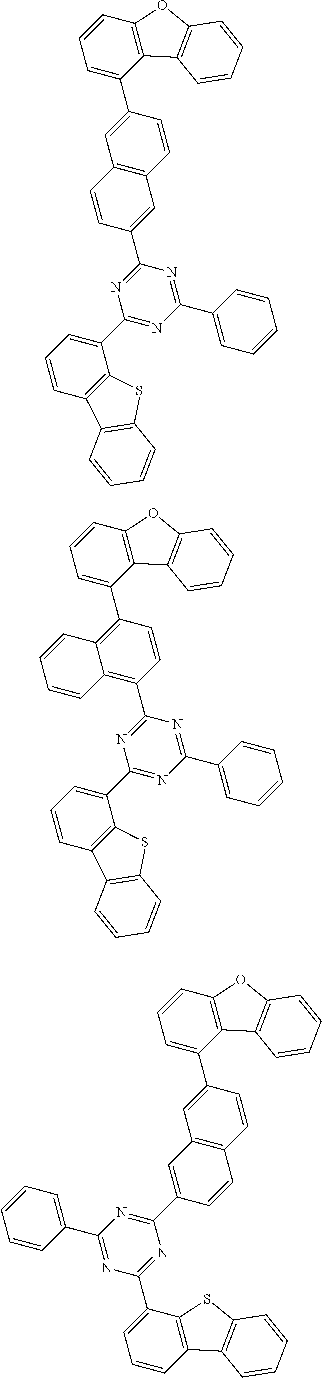

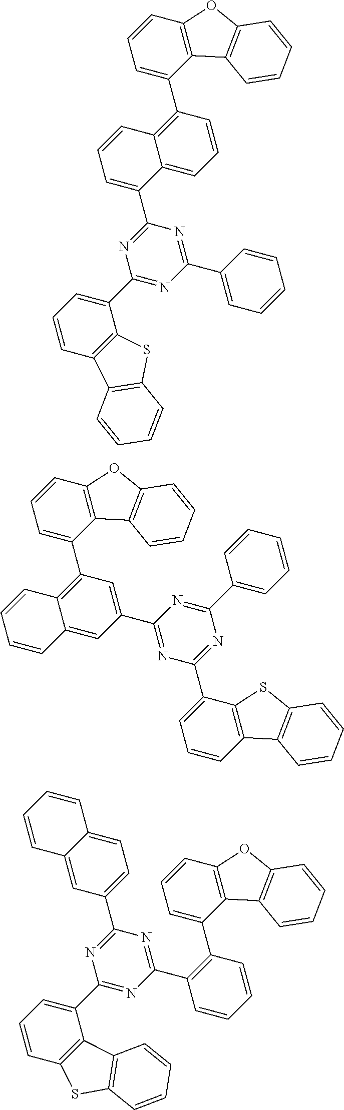

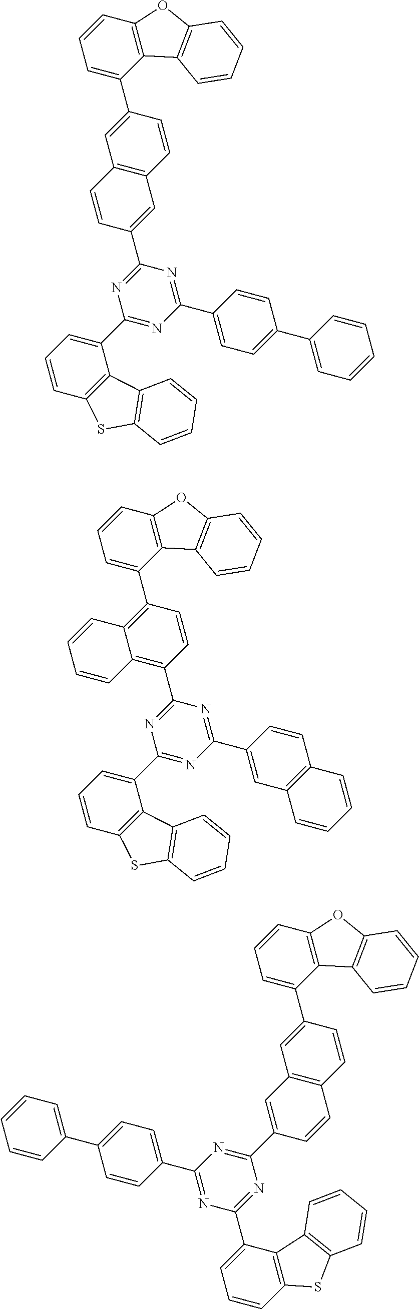 [SMILES: c1ccc(-c2ccc(-c3nc(-c4ccc5cc(-c6cccc7oc8ccccc8c67)ccc5c4)nc(-c4cccc5sc6ccccc6c45)n3)cc2)cc1 ; c1ccc2cc(-c3nc(-c4ccc(-c5cccc6oc7ccccc7c56)c5ccccc45)nc(-c4cccc5sc6ccccc6c45)n3)ccc2c1 ; c1ccc(-c2ccc(-c3nc(-c4ccc5ccc(-c6cccc7oc8ccccc8c67)cc5c4)nc(-c4cccc5sc6ccccc6c45)n3)cc2)cc1]

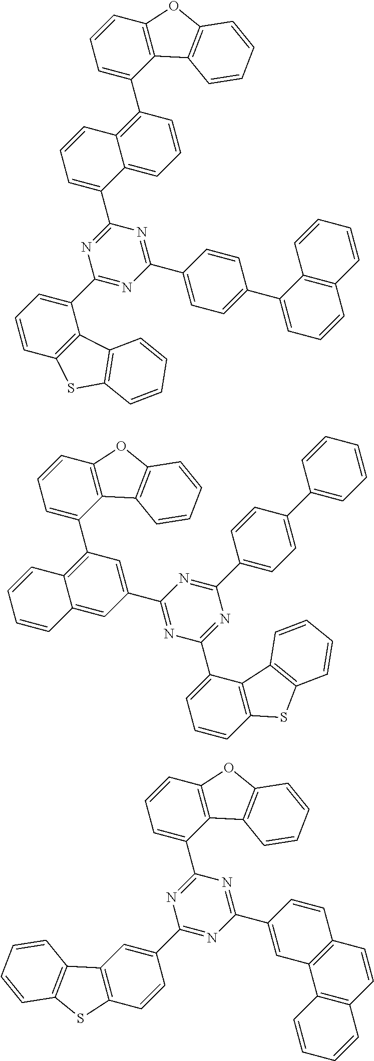

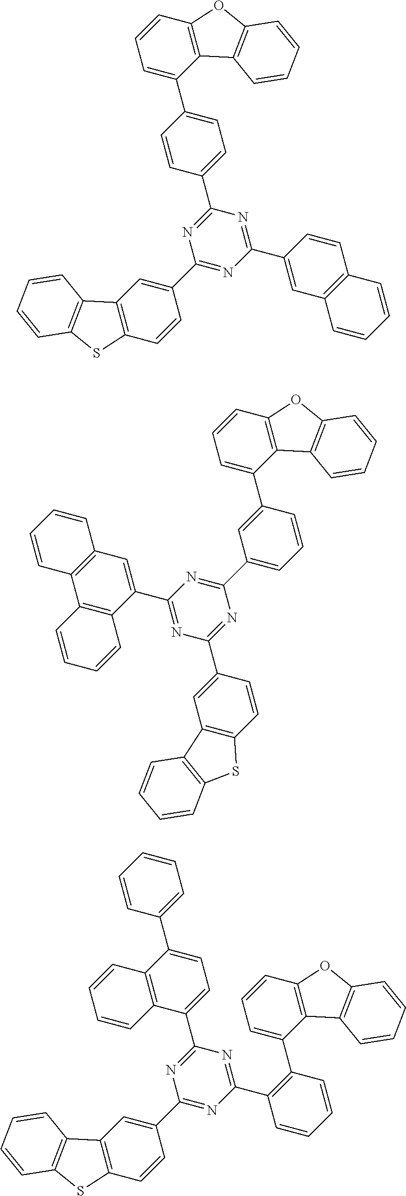

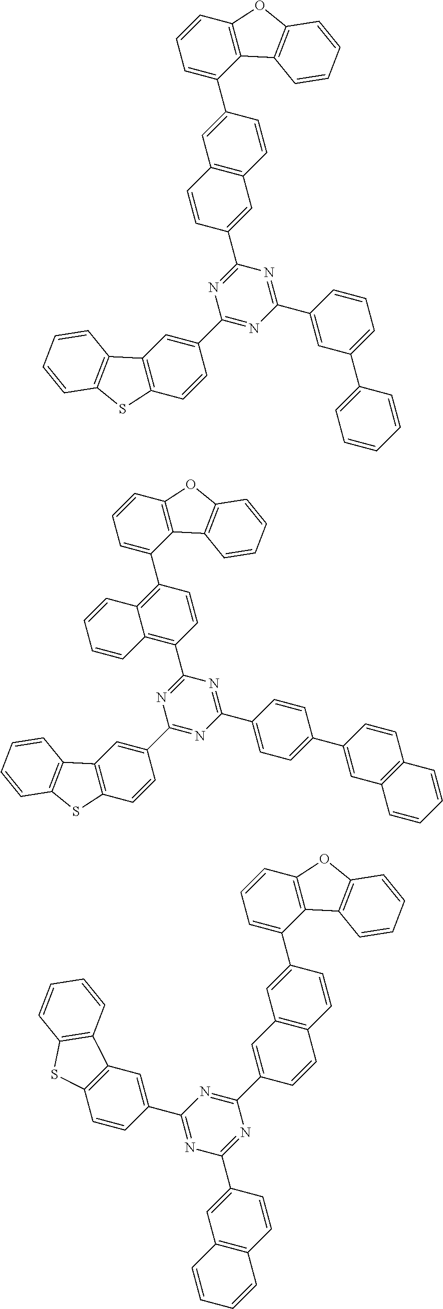 [SMILES: c1ccc(-c2cccc(-c3nc(-c4ccc5cc(-c6cccc7oc8ccccc8c67)ccc5c4)nc(-c4ccc5sc6ccccc6c5c4)n3)c2)cc1 ; c1ccc2cc(-c3ccc(-c4nc(-c5ccc6sc7ccccc7c6c5)nc(-c5ccc(-c6cccc7oc8ccccc8c67)c6ccccc56)n4)cc3)ccc2c1 ; c1ccc2cc(-c3nc(-c4ccc5ccc(-c6cccc7oc8ccccc8c67)cc5c4)nc(-c4ccc5sc6ccccc6c5c4)n3)ccc2c1]

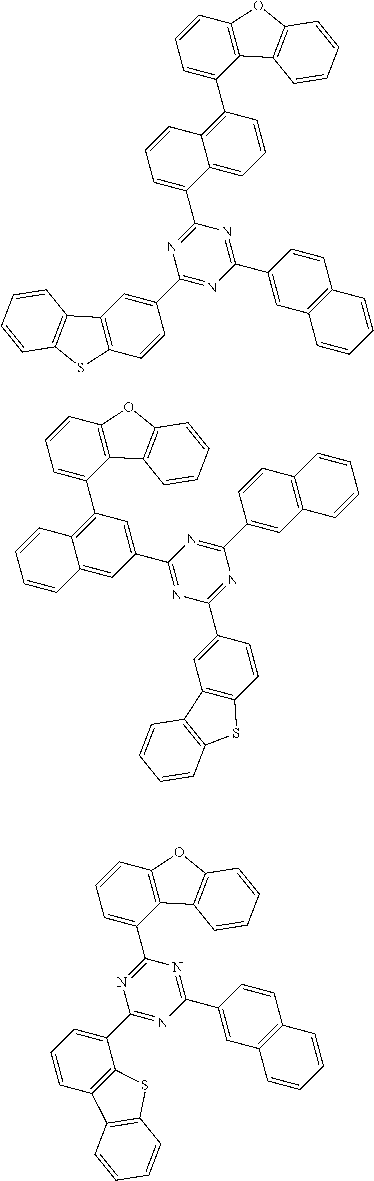 [SMILES: c1ccc2cc(-c3nc(-c4ccc5sc6ccccc6c5c4)nc(-c4cccc5c(-c6cccc7oc8ccccc8c67)cccc45)n3)ccc2c1 ; c1ccc2cc(-c3nc(-c4cc(-c5cccc6oc7ccccc7c56)c5ccccc5c4)nc(-c4ccc5sc6ccccc6c5c4)n3)ccc2c1 ; c1ccc2cc(-c3nc(-c4cccc5c4sc4ccccc45)nc(-c4cccc5oc6ccccc6c45)n3)ccc2c1]

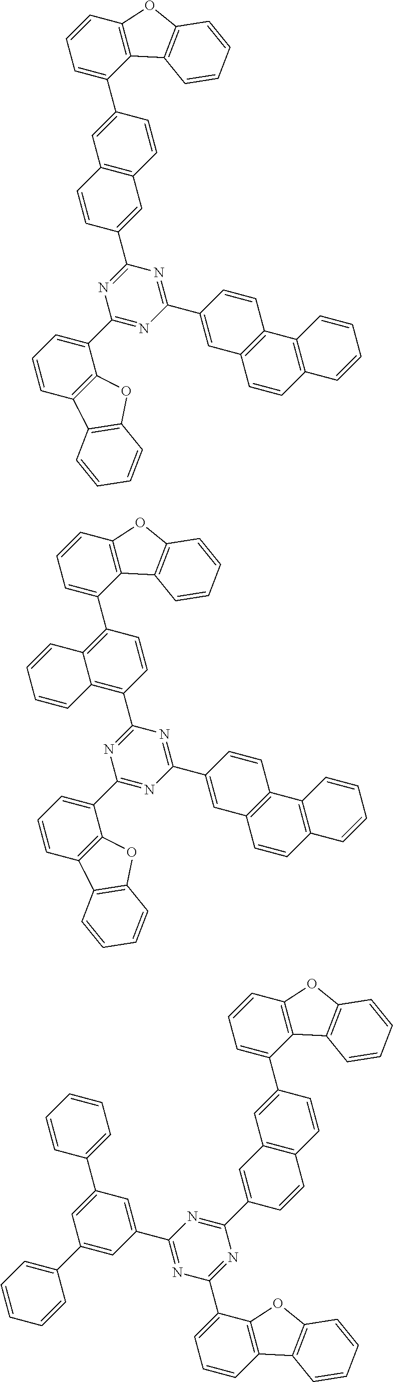

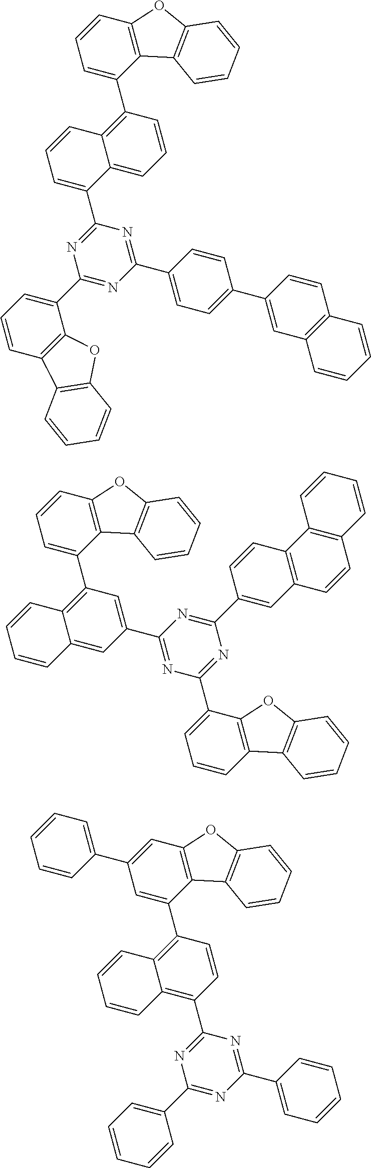

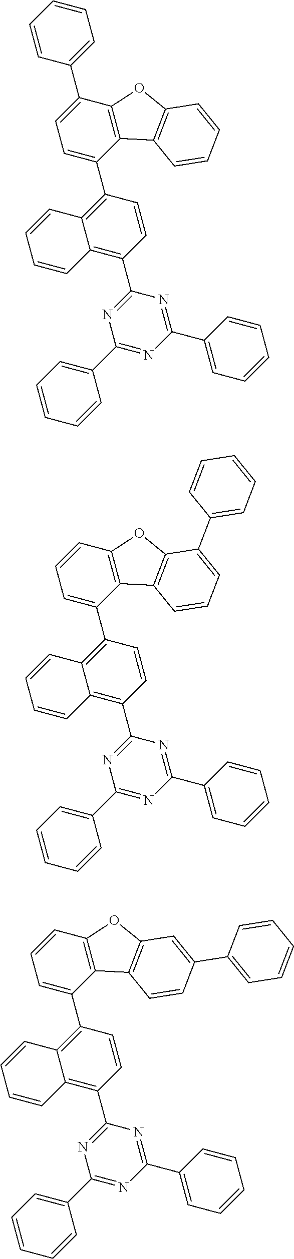 [SMILES: c1ccc(-c2nc(-c3ccccc3)nc(-c3ccc(-c4ccc(-c5ccccc5)c5oc6ccccc6c45)c4ccccc34)n2)cc1 ; c1ccc(-c2nc(-c3ccccc3)nc(-c3ccc(-c4cccc5oc6c(-c7ccccc7)cccc6c45)c4ccccc34)n2)cc1 ; c1ccc(-c2ccc3c(c2)oc2cccc(-c4ccc(-c5nc(-c6ccccc6)nc(-c6ccccc6)n5)c5ccccc45)c23)cc1]

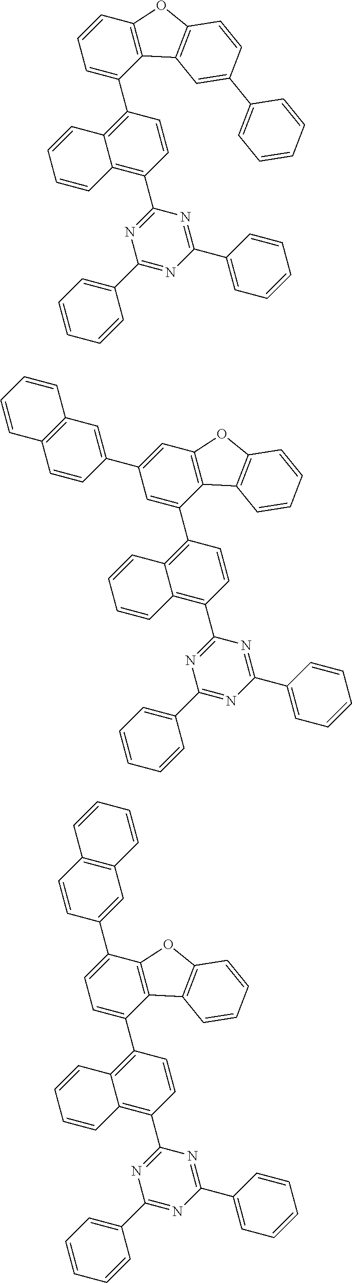 [SMILES: c1ccc(-c2ccc3oc4cccc(-c5ccc(-c6nc(-c7ccccc7)nc(-c7ccccc7)n6)c6ccccc56)c4c3c2)cc1 ; c1ccc(-c2nc(-c3ccccc3)nc(-c3ccc(-c4cc(-c5ccc6ccccc6c5)cc5oc6ccccc6c45)c4ccccc34)n2)cc1 ; c1ccc(-c2nc(-c3ccccc3)nc(-c3ccc(-c4ccc(-c5ccc6ccccc6c5)c5oc6ccccc6c45)c4ccccc34)n2)cc1]

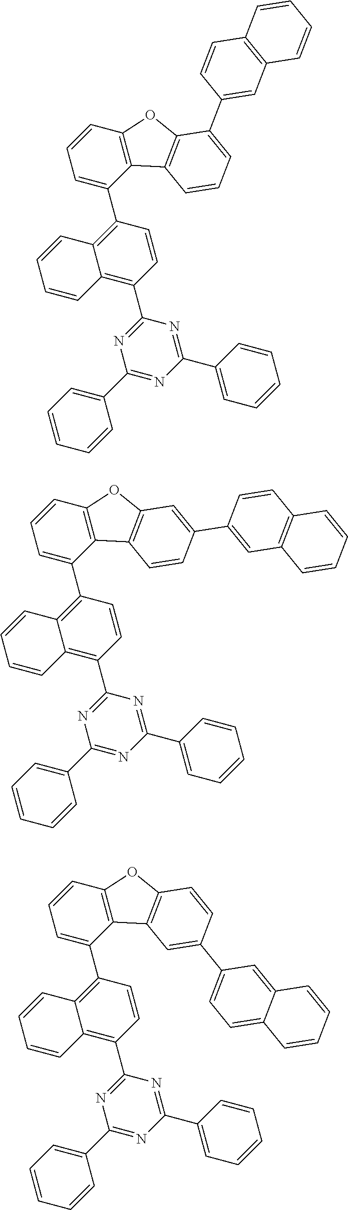

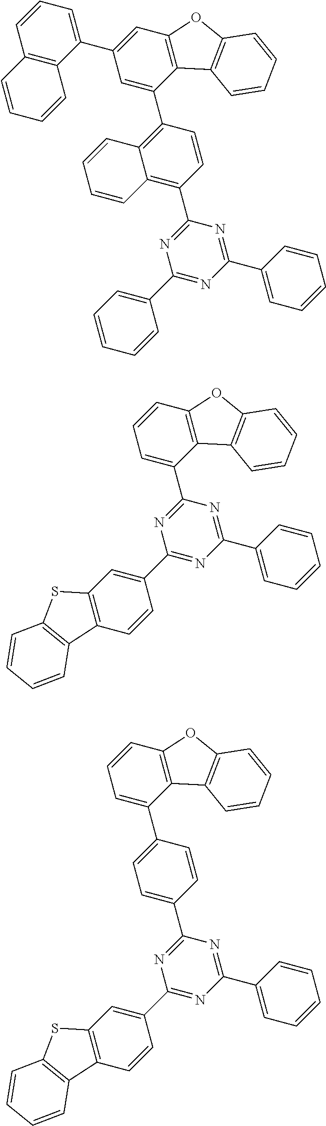 [SMILES: c1ccc(-c2nc(-c3ccccc3)nc(-c3ccc(-c4cc(-c5cccc6ccccc56)cc5oc6ccccc6c45)c4ccccc34)n2)cc1 ; c1ccc(-c2nc(-c3ccc4c(c3)sc3ccccc34)nc(-c3cccc4oc5ccccc5c34)n2)cc1 ; c1ccc(-c2nc(-c3ccc(-c4cccc5oc6ccccc6c45)cc3)nc(-c3ccc4c(c3)sc3ccccc34)n2)cc1]

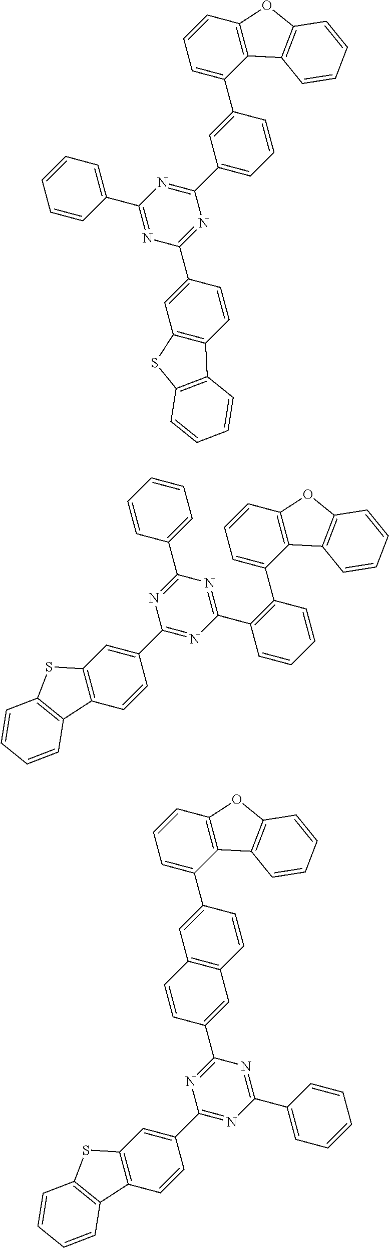

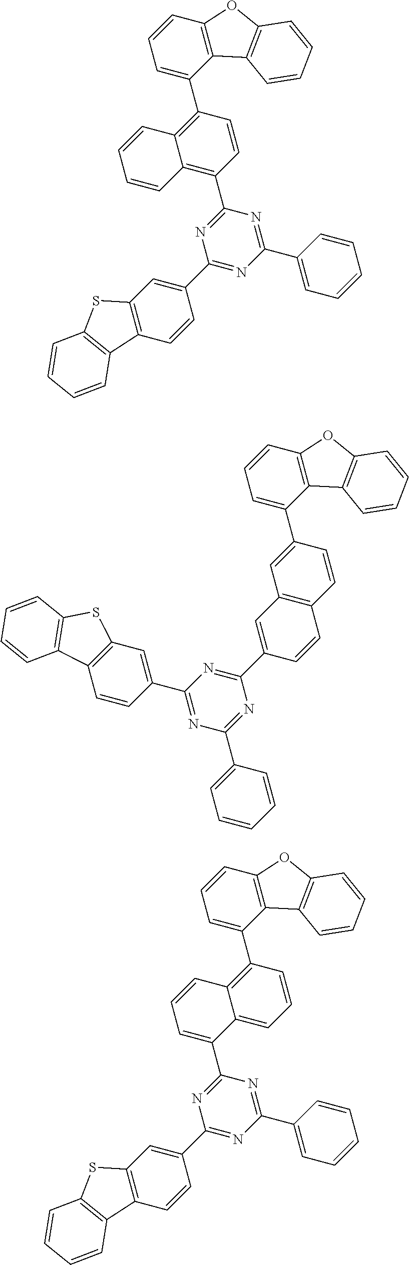

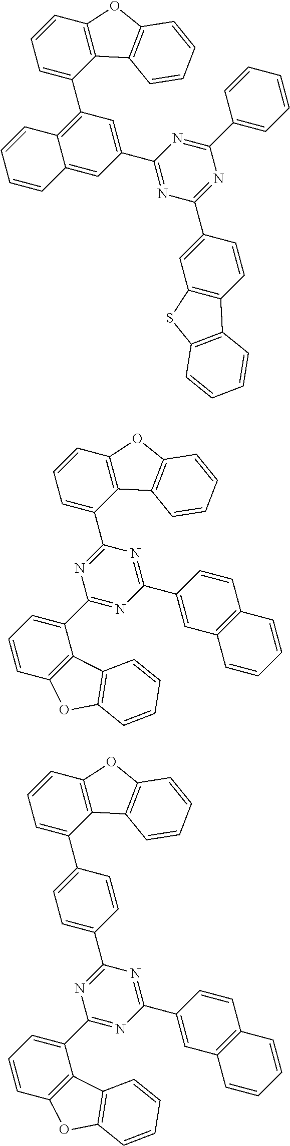

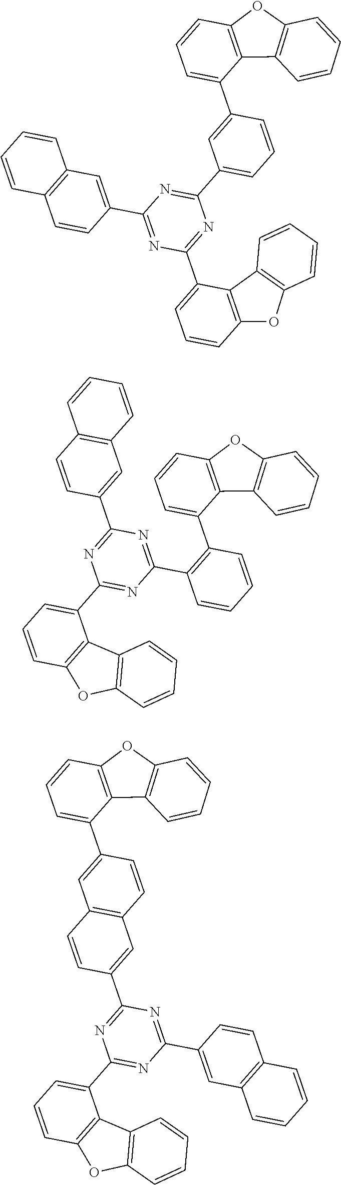

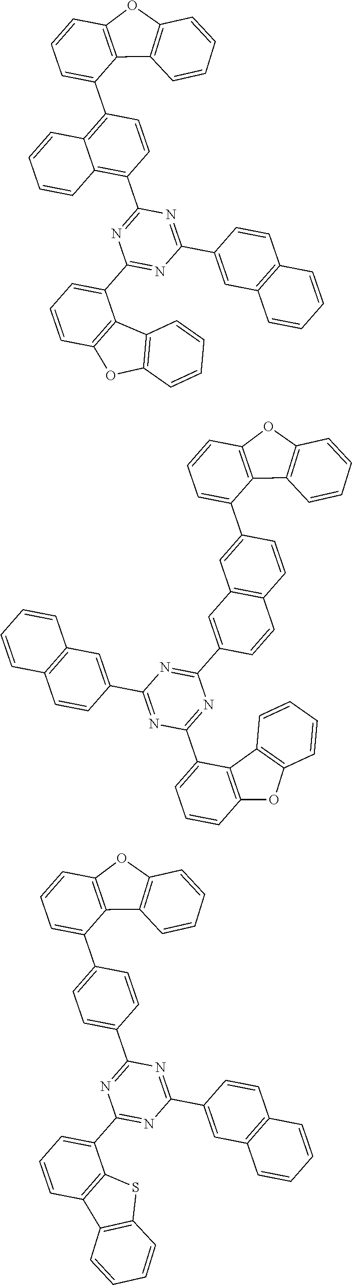 [SMILES: c1ccc2cc(-c3nc(-c4ccc(-c5cccc6oc7ccccc7c56)c5ccccc45)nc(-c4cccc5oc6ccccc6c45)n3)ccc2c1 ; c1ccc2cc(-c3nc(-c4ccc5ccc(-c6cccc7oc8ccccc8c67)cc5c4)nc(-c4cccc5oc6ccccc6c45)n3)ccc2c1 ; c1ccc2cc(-c3nc(-c4ccc(-c5cccc6oc7ccccc7c56)cc4)nc(-c4cccc5c4sc4ccccc45)n3)ccc2c1]

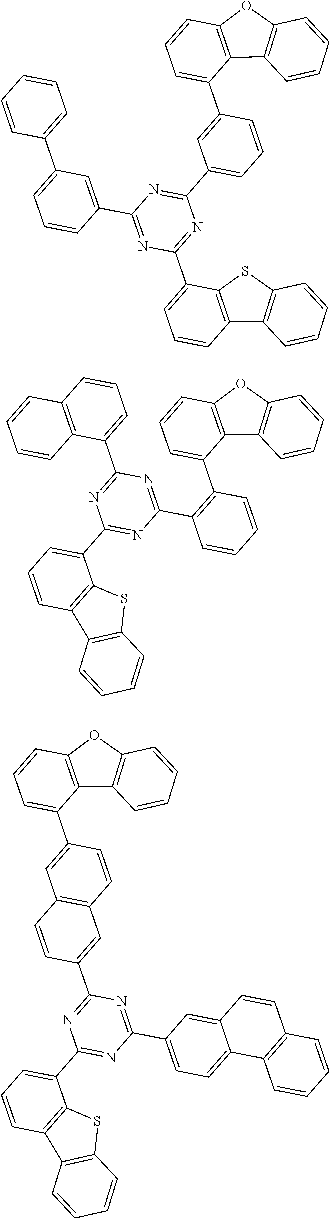

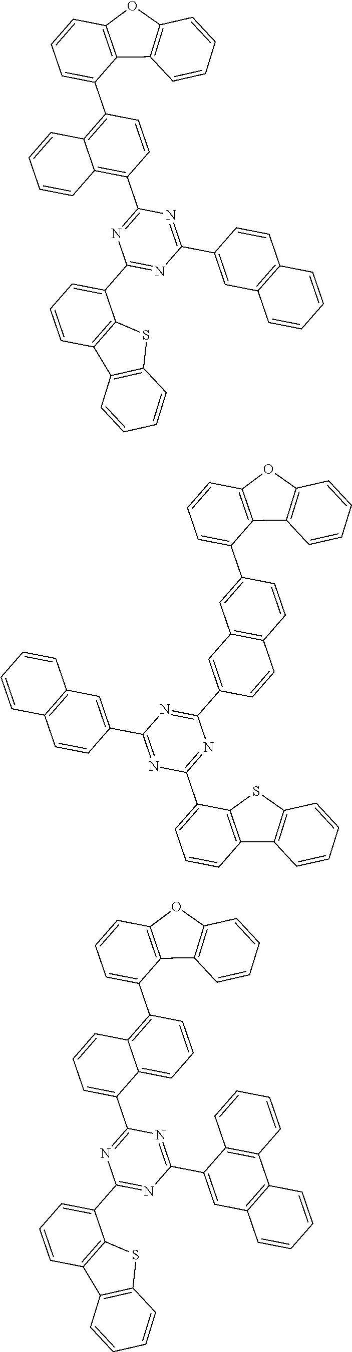 [SMILES: c1ccc2cc(-c3nc(-c4ccc(-c5cccc6oc7ccccc7c56)c5ccccc45)nc(-c4cccc5c4sc4ccccc45)n3)ccc2c1 ; c1ccc2cc(-c3nc(-c4ccc5ccc(-c6cccc7oc8ccccc8c67)cc5c4)nc(-c4cccc5c4sc4ccccc45)n3)ccc2c1 ; c1ccc2c(c1)cc(-c1nc(-c3cccc4c(-c5cccc6oc7ccccc7c56)cccc34)nc(-c3cccc4c3sc3ccccc34)n1)c1ccccc12]

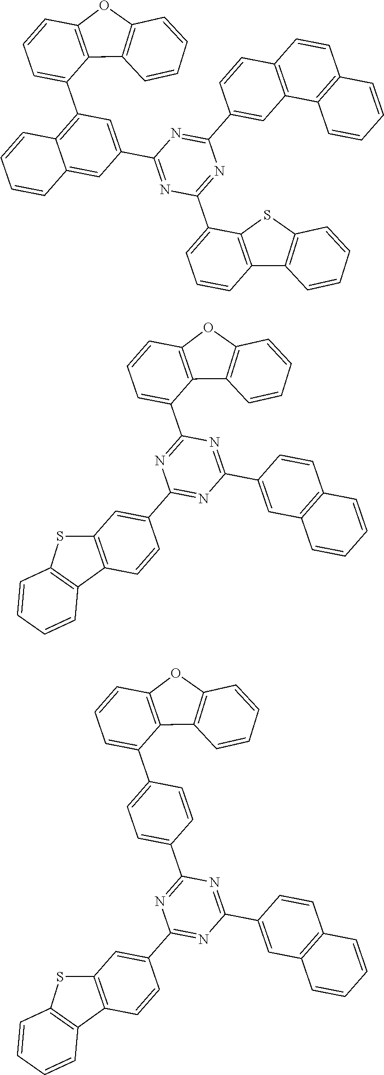

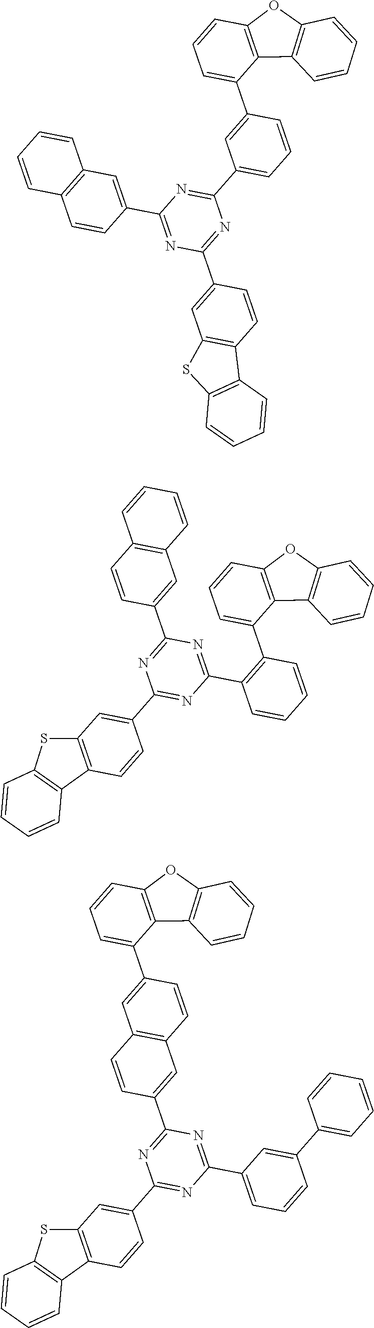 [SMILES: c1cc(-c2nc(-c3ccc4ccccc4c3)nc(-c3ccc4c(c3)sc3ccccc34)n2)cc(-c2cccc3oc4ccccc4c23)c1 ; c1ccc(-c2cccc3oc4ccccc4c23)c(-c2nc(-c3ccc4ccccc4c3)nc(-c3ccc4c(c3)sc3ccccc34)n2)c1 ; c1ccc(-c2cccc(-c3nc(-c4ccc5cc(-c6cccc7oc8ccccc8c67)ccc5c4)nc(-c4ccc5c(c4)sc4ccccc45)n3)c2)cc1]

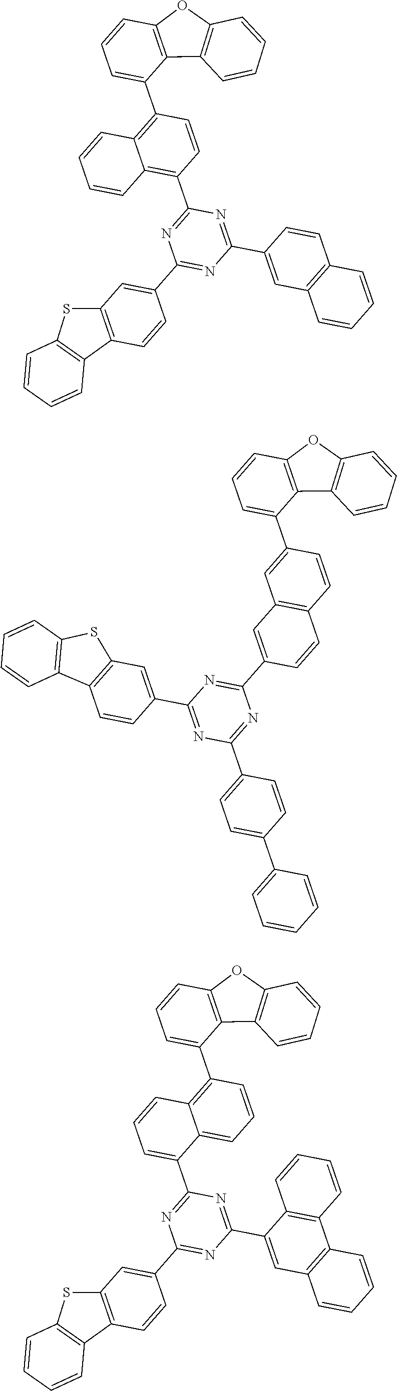 [SMILES: c1ccc2cc(-c3nc(-c4ccc5c(c4)sc4ccccc45)nc(-c4ccc(-c5cccc6oc7ccccc7c56)c5ccccc45)n3)ccc2c1 ; c1ccc(-c2ccc(-c3nc(-c4ccc5ccc(-c6cccc7oc8ccccc8c67)cc5c4)nc(-c4ccc5c(c4)sc4ccccc45)n3)cc2)cc1 ; c1ccc2c(c1)cc(-c1nc(-c3ccc4c(c3)sc3ccccc34)nc(-c3cccc4c(-c5cccc6oc7ccccc7c56)cccc34)n1)c1ccccc12]

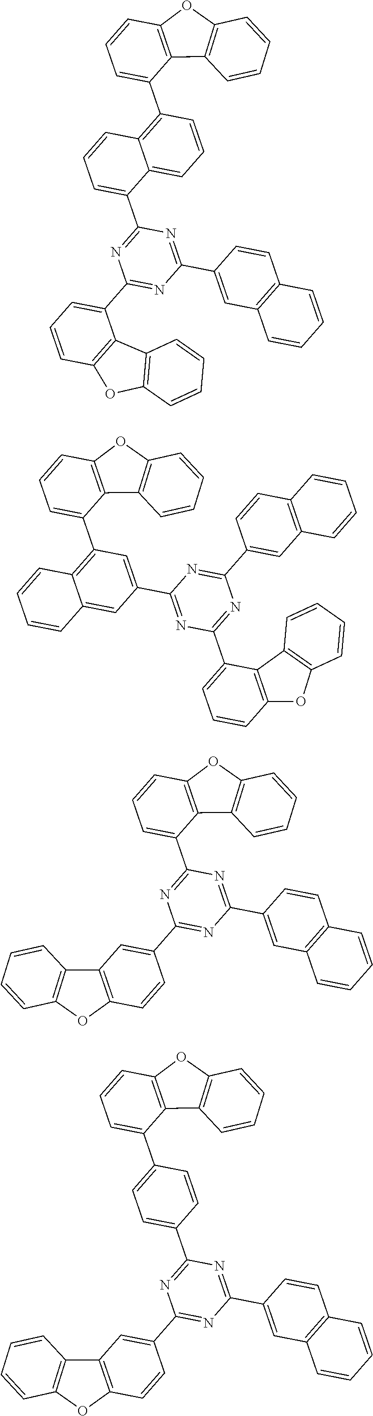

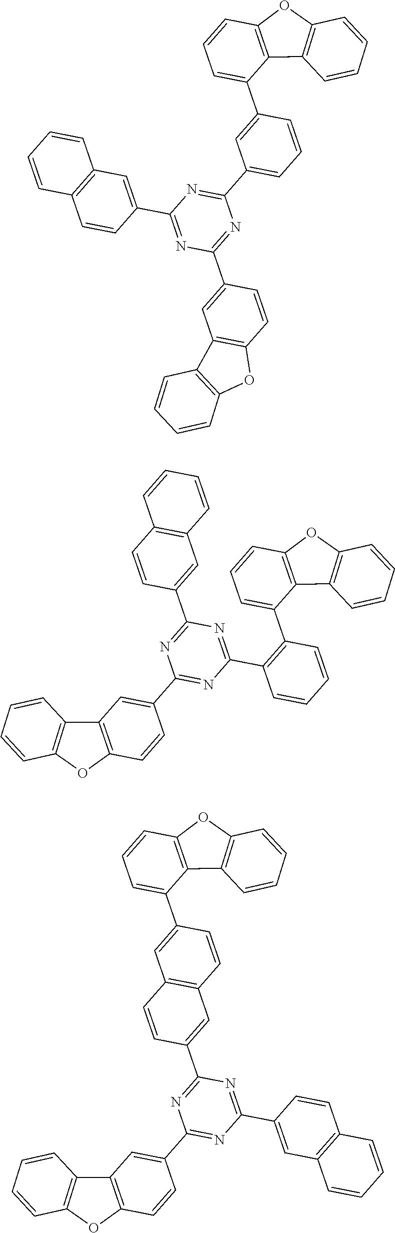

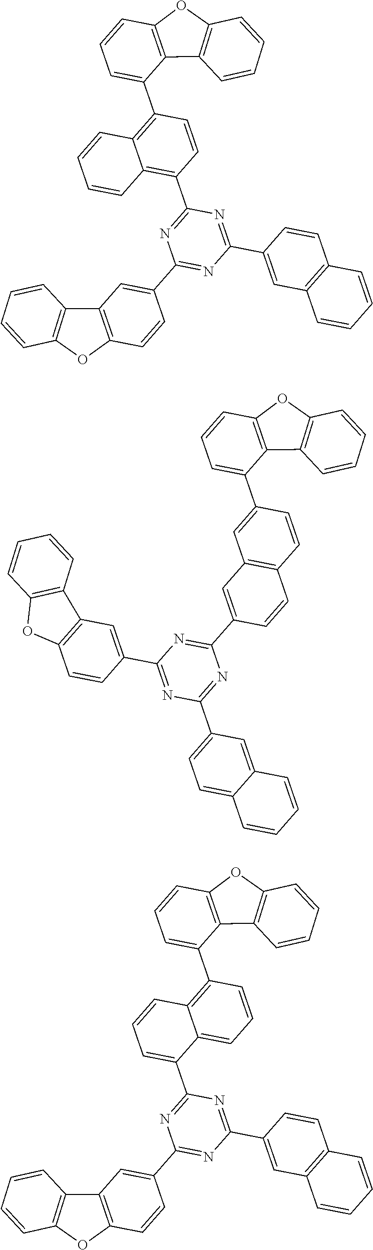

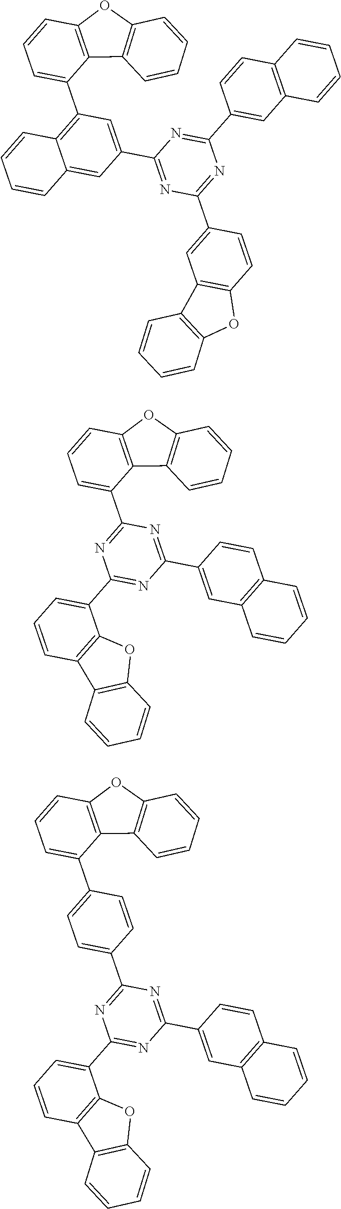

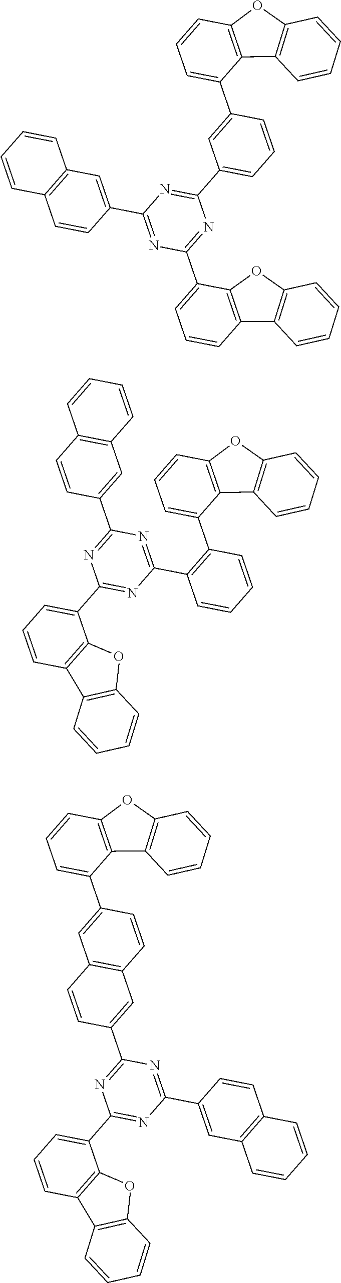 [SMILES: c1cc(-c2nc(-c3ccc4ccccc4c3)nc(-c3cccc4c3oc3ccccc34)n2)cc(-c2cccc3oc4ccccc4c23)c1 ; c1ccc(-c2cccc3oc4ccccc4c23)c(-c2nc(-c3ccc4ccccc4c3)nc(-c3cccc4c3oc3ccccc34)n2)c1 ; c1ccc2cc(-c3nc(-c4ccc5cc(-c6cccc7oc8ccccc8c67)ccc5c4)nc(-c4cccc5c4oc4ccccc45)n3)ccc2c1]

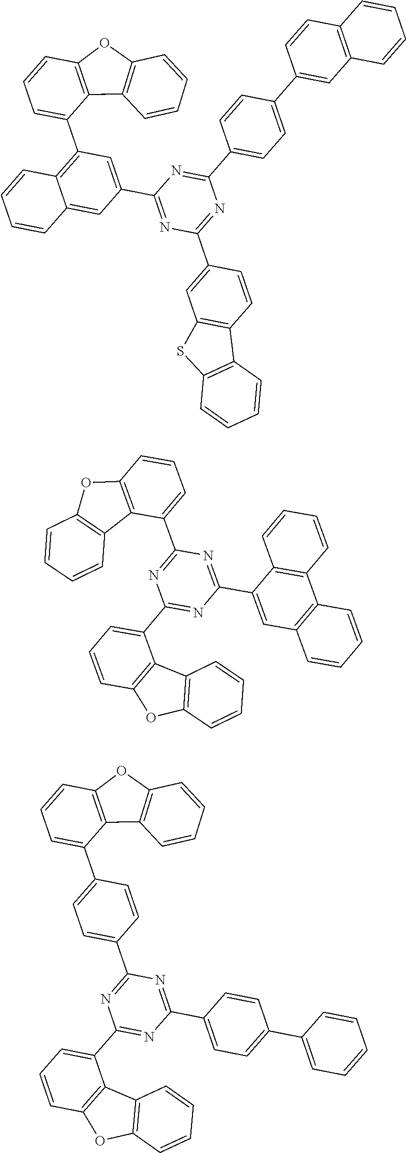 [SMILES: c1ccc2cc(-c3ccc(-c4nc(-c5cc(-c6cccc7oc8ccccc8c67)c6ccccc6c5)nc(-c5ccc6c(c5)sc5ccccc56)n4)cc3)ccc2c1 ; c1ccc2c(c1)cc(-c1nc(-c3cccc4oc5ccccc5c34)nc(-c3cccc4oc5ccccc5c34)n1)c1ccccc12 ; c1ccc(-c2ccc(-c3nc(-c4ccc(-c5cccc6oc7ccccc7c56)cc4)nc(-c4cccc5oc6ccccc6c45)n3)cc2)cc1]

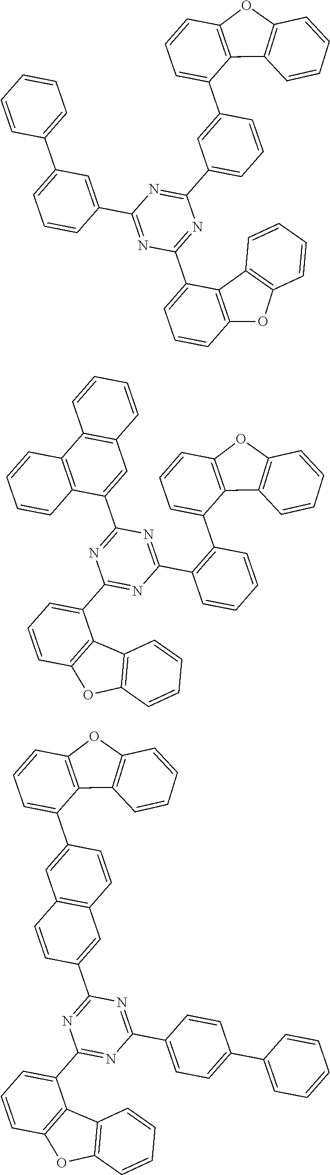

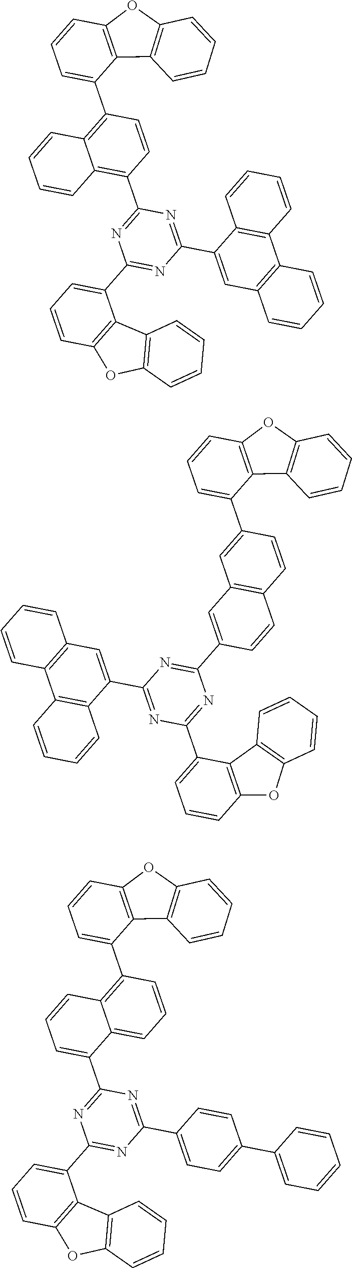 [SMILES: c1ccc2c(c1)cc(-c1nc(-c3ccc(-c4cccc5oc6ccccc6c45)c4ccccc34)nc(-c3cccc4oc5ccccc5c34)n1)c1ccccc12 ; c1ccc2c(c1)cc(-c1nc(-c3ccc4ccc(-c5cccc6oc7ccccc7c56)cc4c3)nc(-c3cccc4oc5ccccc5c34)n1)c1ccccc12 ; c1ccc(-c2ccc(-c3nc(-c4cccc5c(-c6cccc7oc8ccccc8c67)cccc45)nc(-c4cccc5oc6ccccc6c45)n3)cc2)cc1]

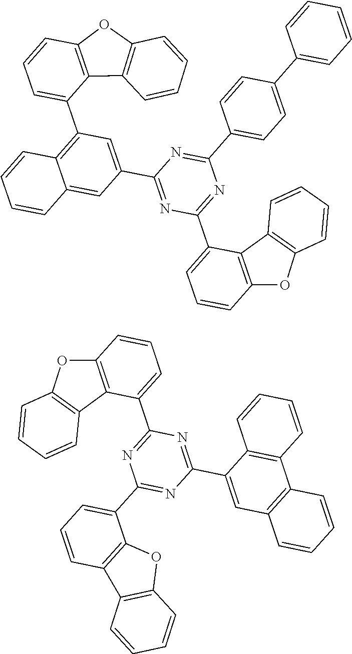 [SMILES: c1ccc(-c2ccc(-c3nc(-c4cc(-c5cccc6oc7ccccc7c56)c5ccccc5c4)nc(-c4cccc5oc6ccccc6c45)n3)cc2)cc1 ; c1ccc2c(c1)cc(-c1nc(-c3cccc4c3oc3ccccc34)nc(-c3cccc4oc5ccccc5c34)n1)c1ccccc12]

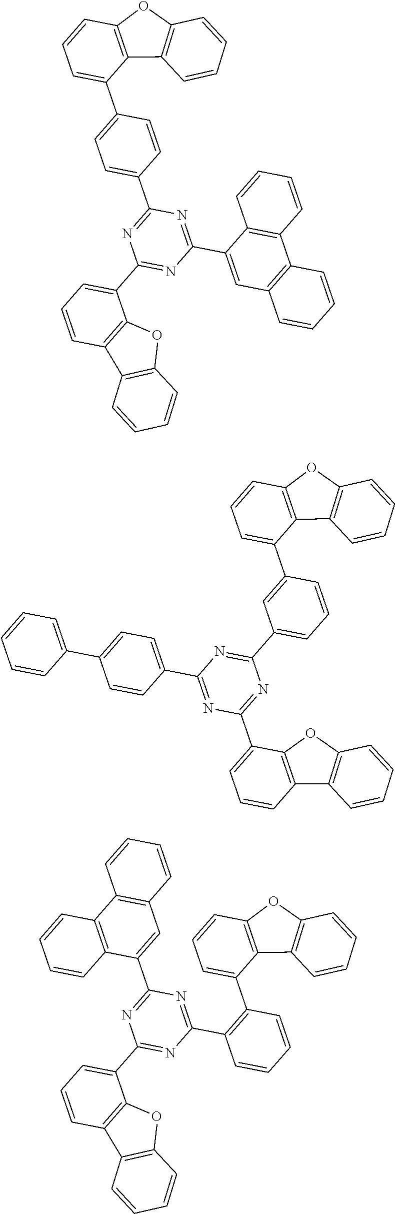 [SMILES: c1ccc2c(c1)cc(-c1nc(-c3ccc(-c4cccc5oc6ccccc6c45)cc3)nc(-c3cccc4c3oc3ccccc34)n1)c1ccccc12 ; c1ccc(-c2ccc(-c3nc(-c4cccc(-c5cccc6oc7ccccc7c56)c4)nc(-c4cccc5c4oc4ccccc45)n3)cc2)cc1 ; c1ccc(-c2cccc3oc4ccccc4c23)c(-c2nc(-c3cc4ccccc4c4ccccc34)nc(-c3cccc4c3oc3ccccc34)n2)c1]

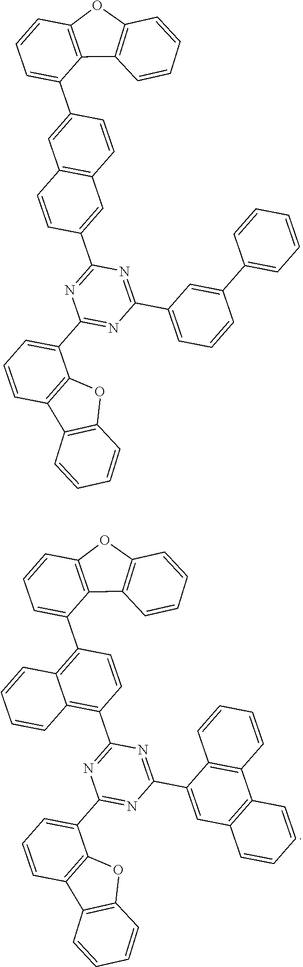 [SMILES: c1ccc(-c2cccc(-c3nc(-c4ccc5cc(-c6cccc7oc8ccccc8c67)ccc5c4)nc(-c4cccc5c4oc4ccccc45)n3)c2)cc1 ; c1ccc2c(c1)cc(-c1nc(-c3ccc(-c4cccc5oc6ccccc6c45)c4ccccc34)nc(-c3cccc4c3oc3ccccc34)n1)c1ccccc12]

The compound of Chemical Formula 1 can be prepared by, for example, a preparation method as shown in Reaction Scheme 1 below.

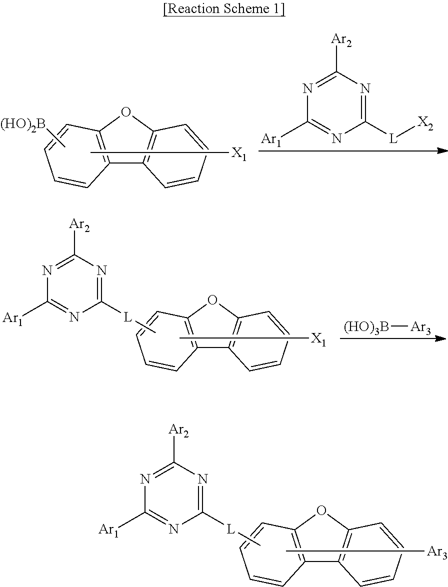

in the Reaction Scheme 1, the definitions of other substituents except for X₁ and X₂ are the same as described above. X₁ and X₂ are each independently halogen, and more preferably bromo or chloro.

The Reaction Scheme 1 is a Suzuki coupling reaction, and preferably performed in the presence of a palladium catalyst and a base, and the reactor for the Suzuki coupling reaction can be changed as known in the art. The method for preparing the compound of Chemical Formula 1 can be more specifically described in the Preparation Examples described below.

Chemical Formula 2 can be any one selected from the group consisting of the following Chemical Formulae 2-1 to 2-3:

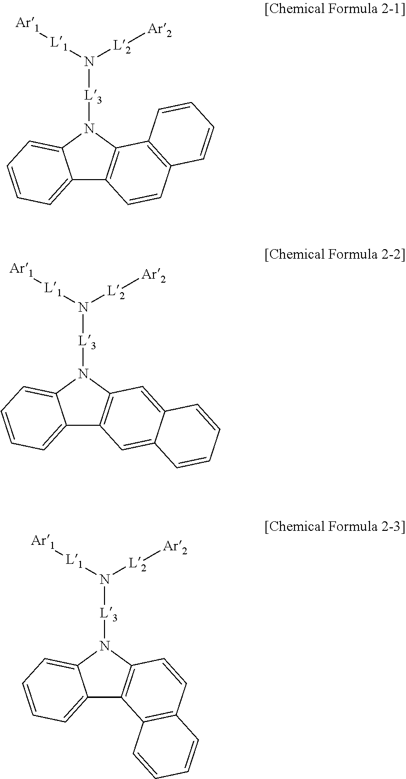

wherein in Chemical Formulae 2-1 to 2-3:

Ar′₁, Ar′₂, and L′₁ to L′₃ are as defined in the Chemical Formula 2.

One or more hydrogens of Chemical Formula 2 can be substituted with deuterium.

Preferably, L′₃ is a substituted or unsubstituted C₆₋₂₀ arylene, or substituted or unsubstituted C₂₋₂₀ heteroarylene containing at least one heteroatom selected from the group consisting of N, O and S. More preferably, L′₃ is any one structural formulae selected from the group consisting of the following structural formulae:

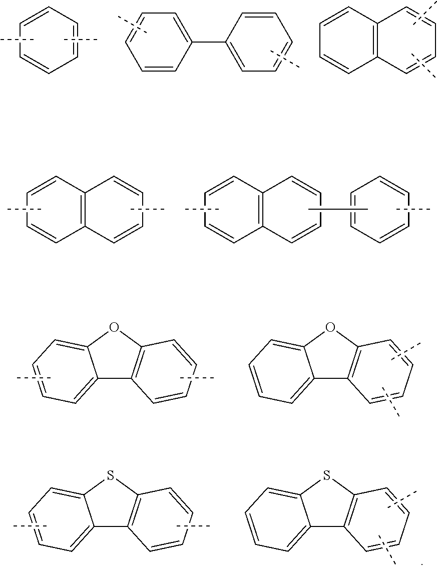

Preferably, L′₁ and L′₂ are each independently a single bond or substituted or unsubstituted C₆₋₂₀ arylene. More preferably, L′₁ and L′₂ are each independently a single bond or phenylene.

Preferably, Ar′₁ and Ar′₂ are each independently a substituted or unsubstituted C₆₋₂₀ aryl, or substituted or unsubstituted C₂₋₂₀ heteroaryl containing at least one heteroatom selected from the group consisting of N, O and S.

Preferably, Ar′₁ and Ar′₂ are each independently phenyl, biphenylyl, terphenylyl, naphthyl, phenanthrenyl, dibenzofuranyl, dibenzothiophenyl, or benzonaphthofuranyl.

Representative examples of the compound of Chemical Formula 2 are as follows:

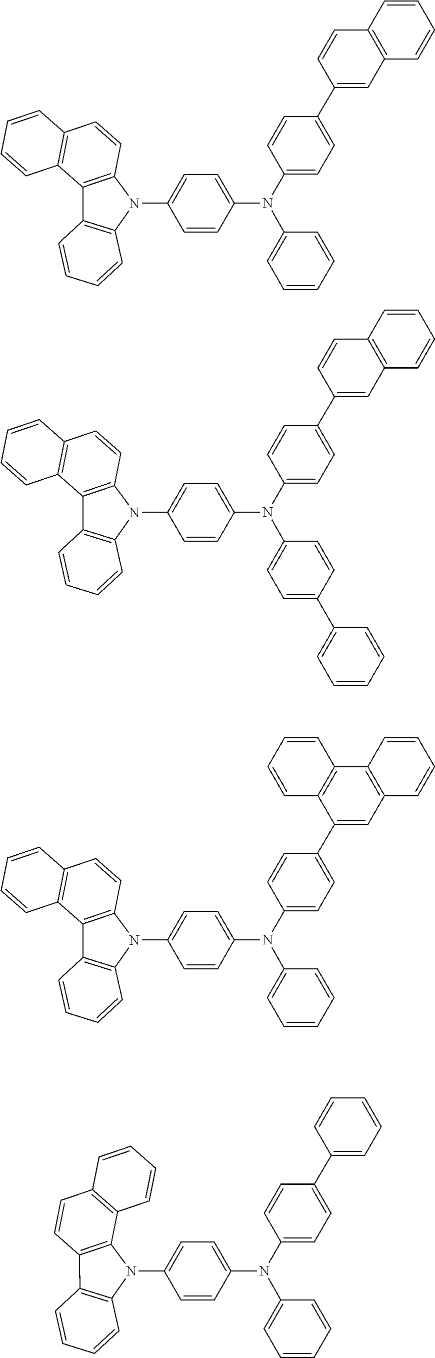 [SMILES: c1ccc(N(c2ccc(-c3ccc4ccccc4c3)cc2)c2ccc(-n3c4ccccc4c4c5ccccc5ccc43)cc2)cc1 ; c1ccc(-c2ccc(N(c3ccc(-c4ccc5ccccc5c4)cc3)c3ccc(-n4c5ccccc5c5c6ccccc6ccc54)cc3)cc2)cc1 ; c1ccc(N(c2ccc(-c3cc4ccccc4c4ccccc34)cc2)c2ccc(-n3c4ccccc4c4c5ccccc5ccc43)cc2)cc1 ; c1ccc(-c2ccc(N(c3ccccc3)c3ccc(-n4c5ccccc5c5ccc6ccccc6c54)cc3)cc2)cc1]

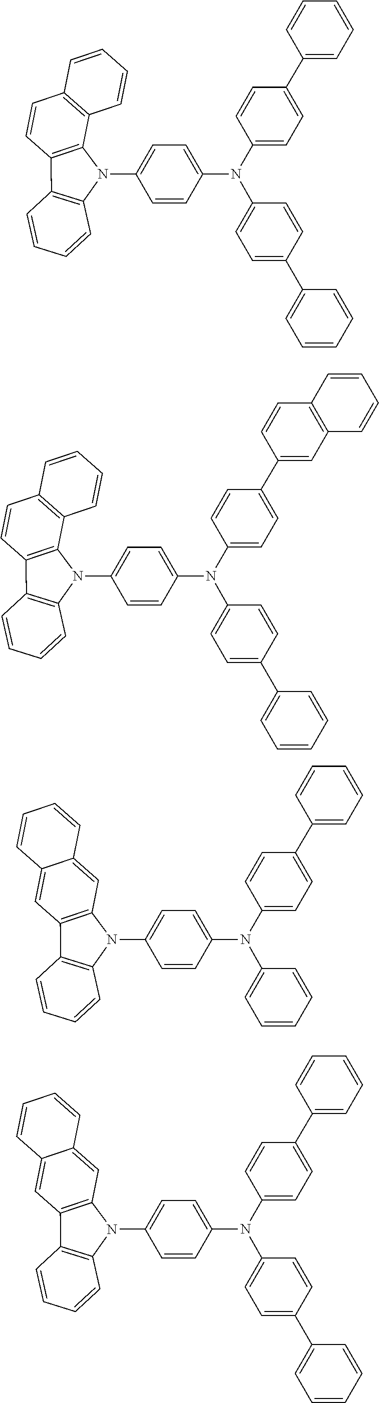

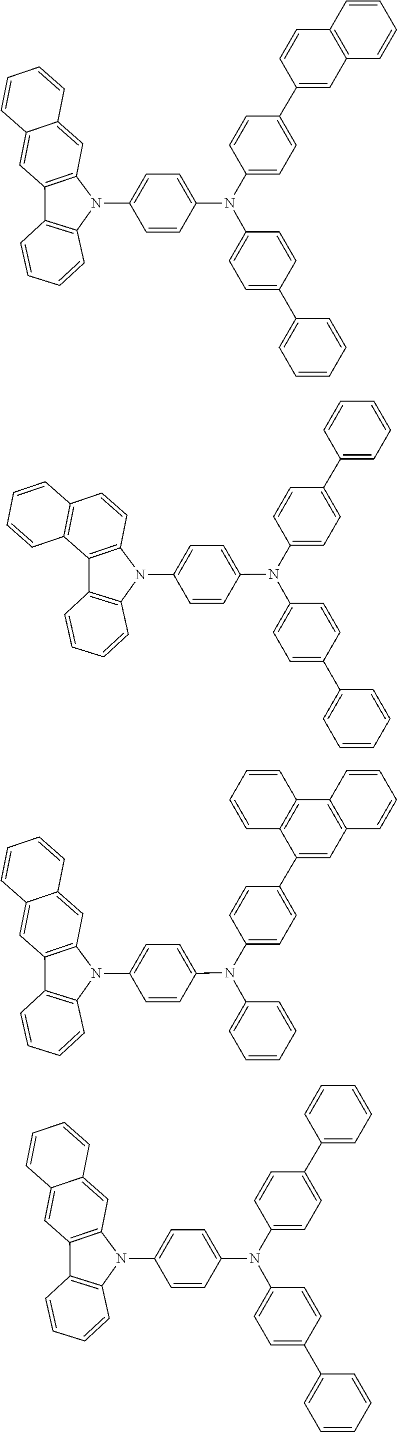

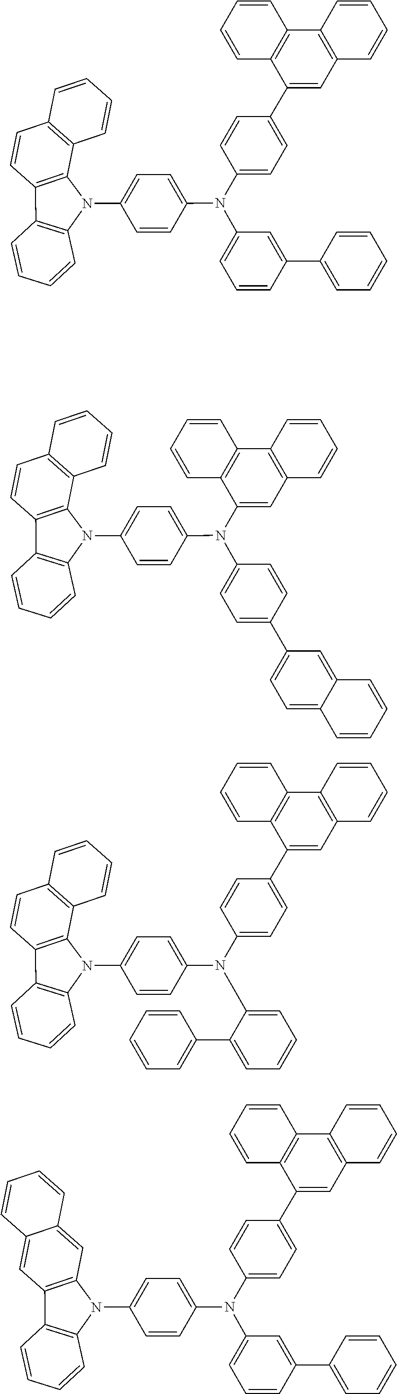

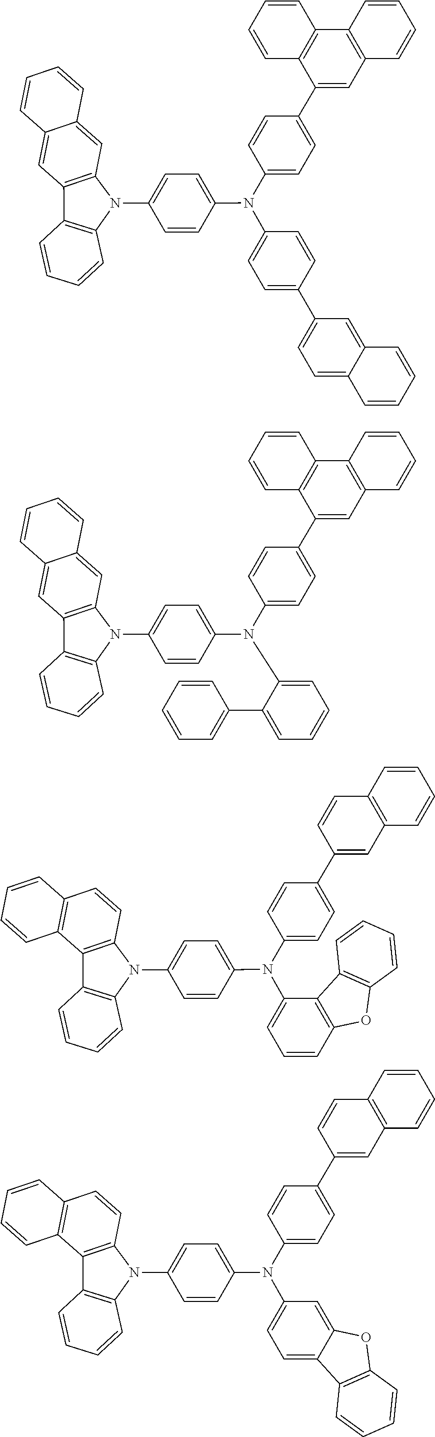

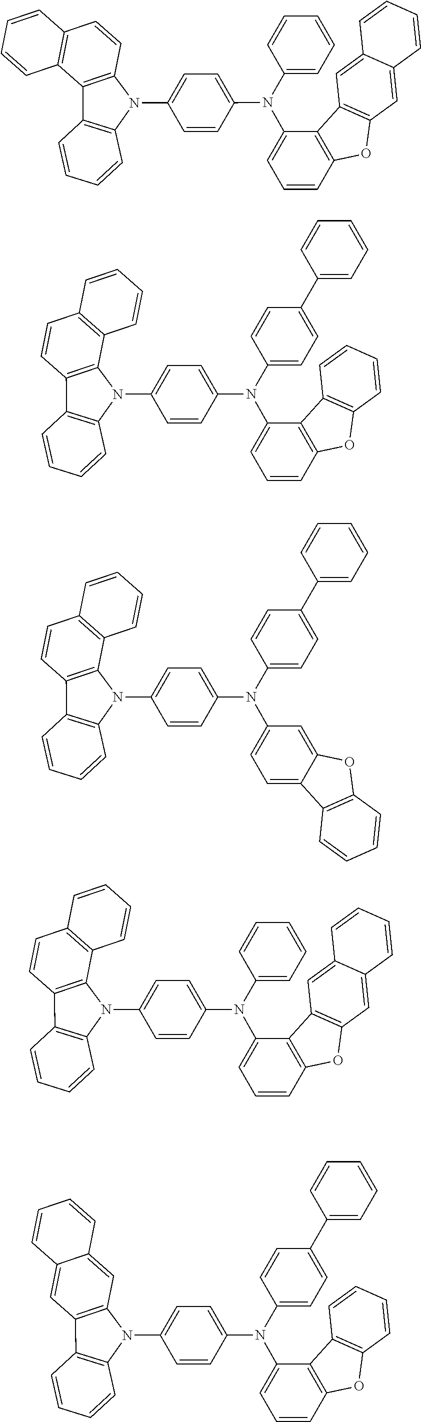

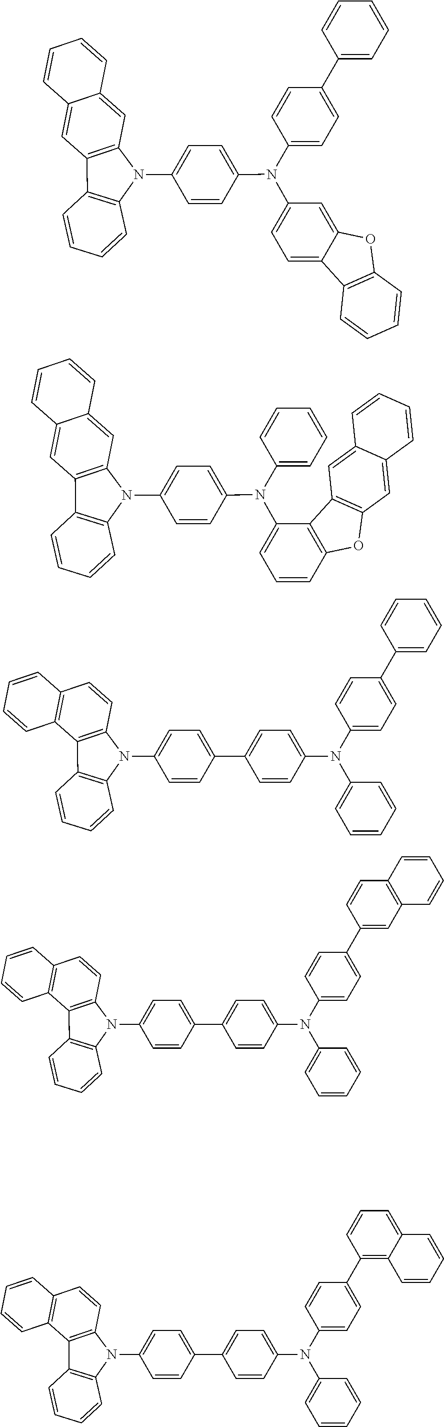

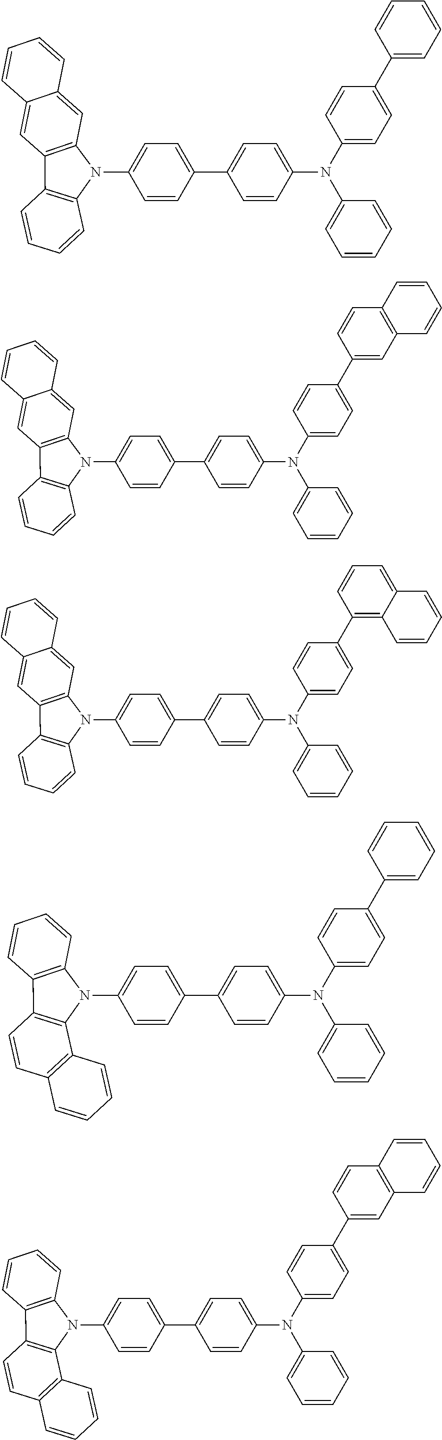

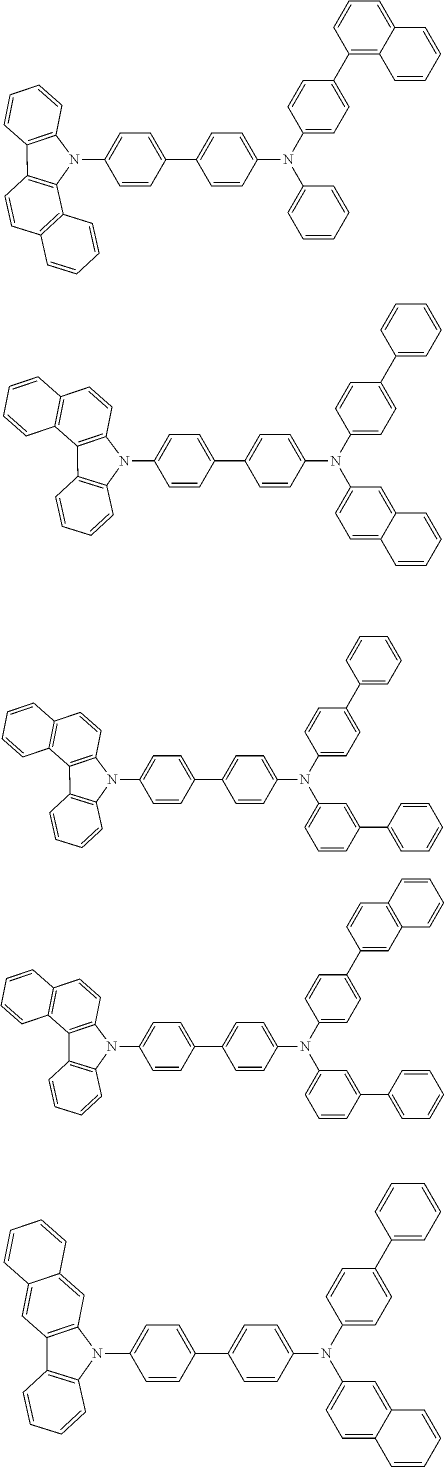

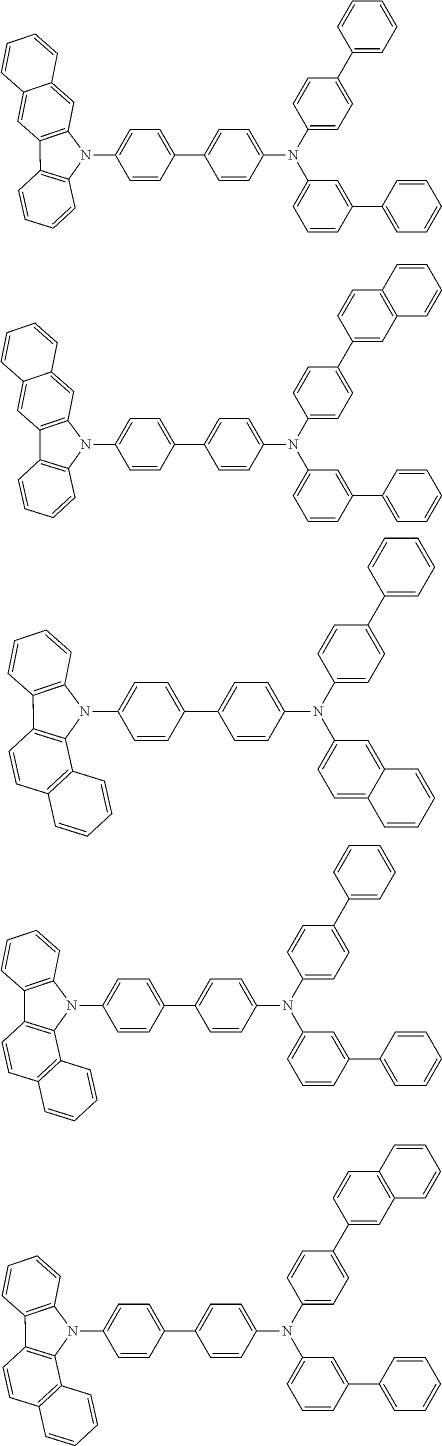

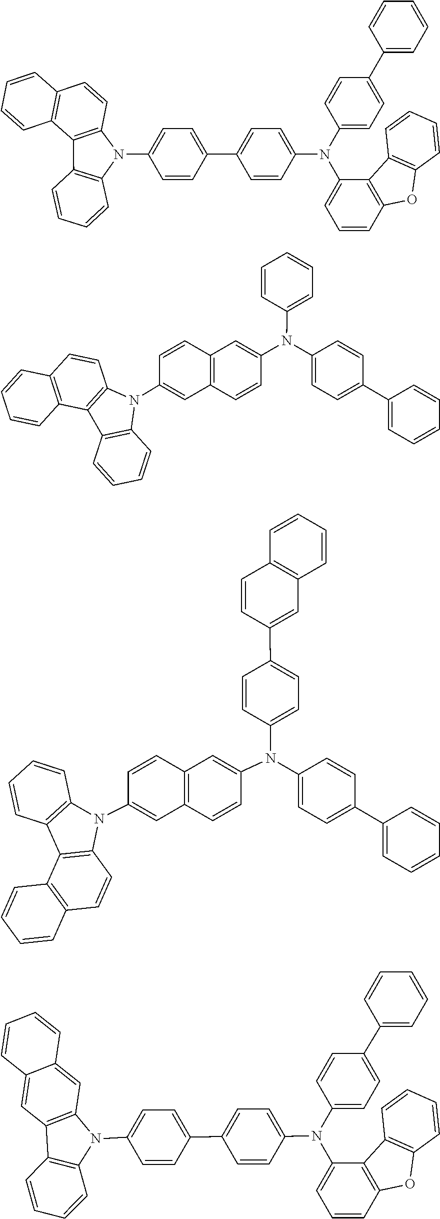 [SMILES: c1ccc(-c2ccc(N(c3ccc(-c4ccc(-n5c6ccccc6c6c7ccccc7ccc65)cc4)cc3)c3cccc4oc5ccccc5c34)cc2)cc1 ; c1ccc(-c2ccc(N(c3ccccc3)c3ccc4cc(-n5c6ccccc6c6c7ccccc7ccc65)ccc4c3)cc2)cc1 ; c1ccc(-c2ccc(N(c3ccc(-c4ccc5ccccc5c4)cc3)c3ccc4cc(-n5c6ccccc6c6c7ccccc7ccc65)ccc4c3)cc2)cc1 ; c1ccc(-c2ccc(N(c3ccc(-c4ccc(-n5c6ccccc6c6cc7ccccc7cc65)cc4)cc3)c3cccc4oc5ccccc5c34)cc2)cc1]

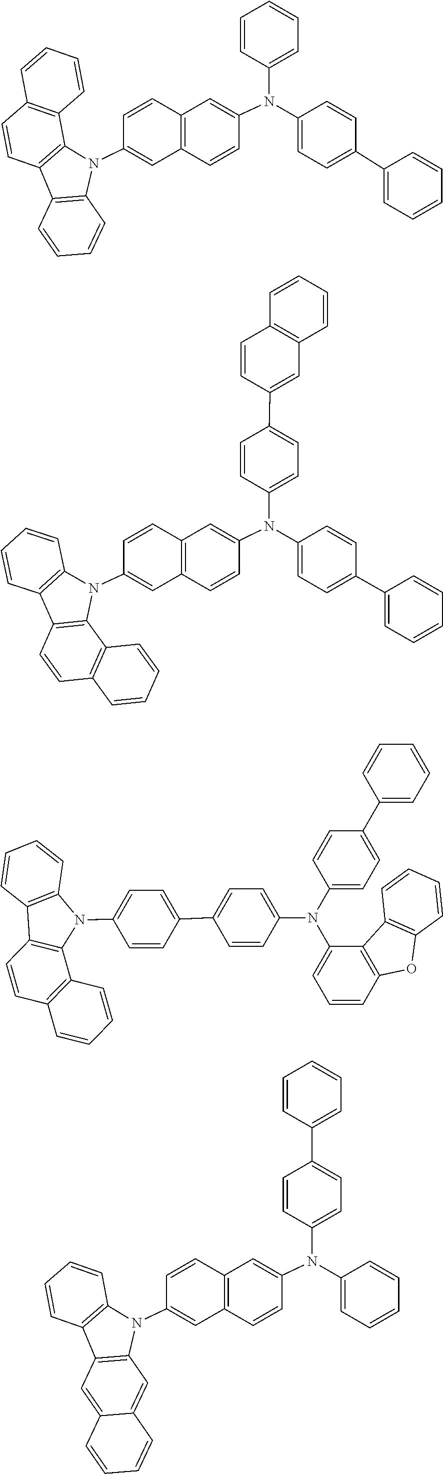 [SMILES: c1ccc(-c2ccc(N(c3ccccc3)c3ccc4cc(-n5c6ccccc6c6ccc7ccccc7c65)ccc4c3)cc2)cc1 ; c1ccc(-c2ccc(N(c3ccc(-c4ccc5ccccc5c4)cc3)c3ccc4cc(-n5c6ccccc6c6ccc7ccccc7c65)ccc4c3)cc2)cc1 ; c1ccc(-c2ccc(N(c3ccc(-c4ccc(-n5c6ccccc6c6ccc7ccccc7c65)cc4)cc3)c3cccc4oc5ccccc5c34)cc2)cc1 ; c1ccc(-c2ccc(N(c3ccccc3)c3ccc4cc(-n5c6ccccc6c6cc7ccccc7cc65)ccc4c3)cc2)cc1]

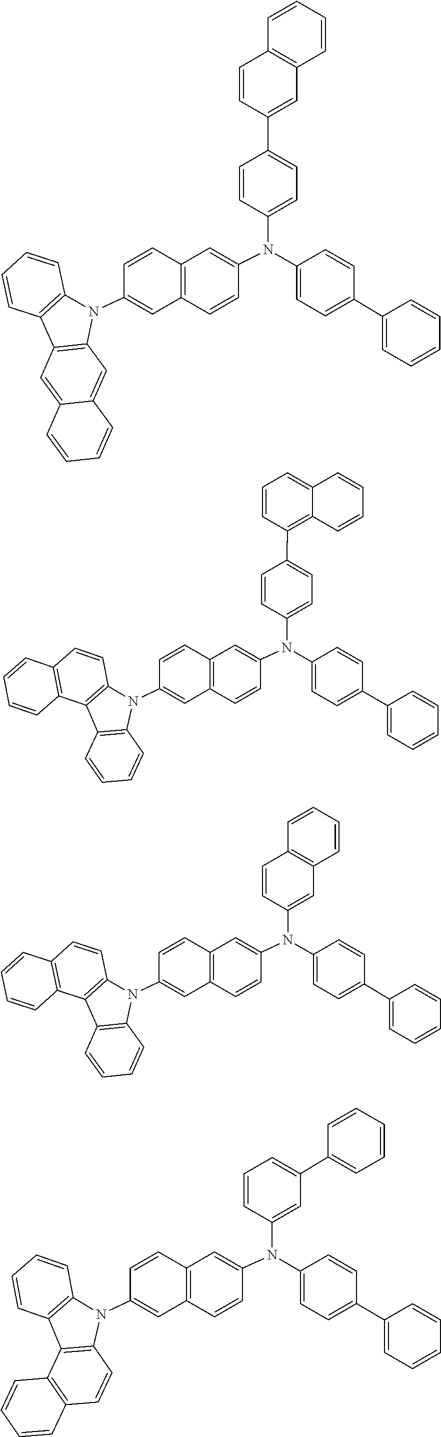 [SMILES: c1ccc(-c2ccc(N(c3ccc(-c4ccc5ccccc5c4)cc3)c3ccc4cc(-n5c6ccccc6c6cc7ccccc7cc65)ccc4c3)cc2)cc1 ; c1ccc(-c2ccc(N(c3ccc(-c4cccc5ccccc45)cc3)c3ccc4cc(-n5c6ccccc6c6c7ccccc7ccc65)ccc4c3)cc2)cc1 ; c1ccc(-c2ccc(N(c3ccc4ccccc4c3)c3ccc4cc(-n5c6ccccc6c6c7ccccc7ccc65)ccc4c3)cc2)cc1 ; c1ccc(-c2ccc(N(c3cccc(-c4ccccc4)c3)c3ccc4cc(-n5c6ccccc6c6c7ccccc7ccc65)ccc4c3)cc2)cc1]

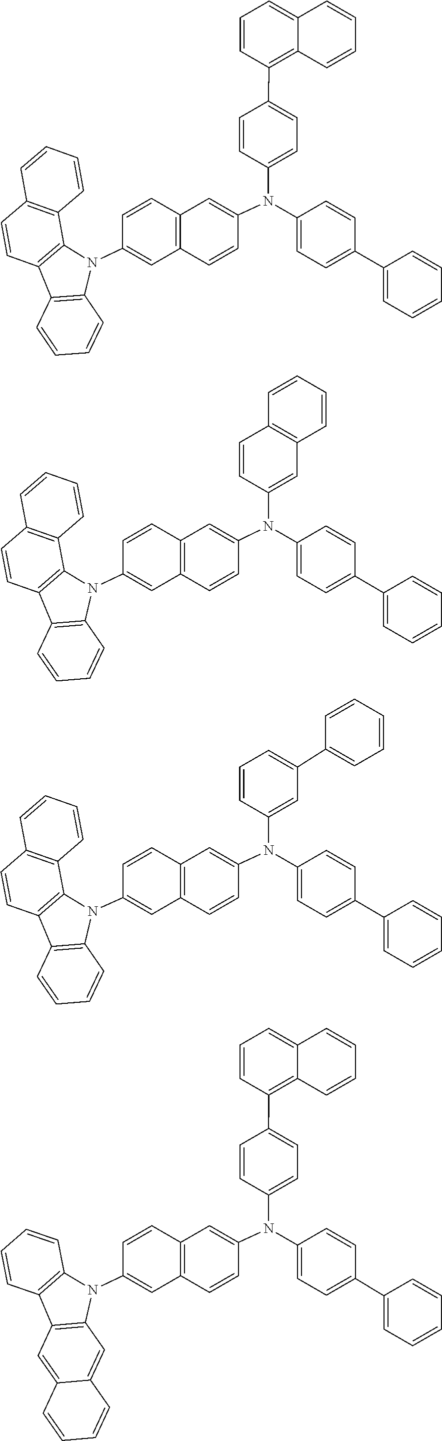 [SMILES: c1ccc(-c2ccc(N(c3ccc(-c4cccc5ccccc45)cc3)c3ccc4cc(-n5c6ccccc6c6ccc7ccccc7c65)ccc4c3)cc2)cc1 ; c1ccc(-c2ccc(N(c3ccc4ccccc4c3)c3ccc4cc(-n5c6ccccc6c6ccc7ccccc7c65)ccc4c3)cc2)cc1 ; c1ccc(-c2ccc(N(c3cccc(-c4ccccc4)c3)c3ccc4cc(-n5c6ccccc6c6ccc7ccccc7c65)ccc4c3)cc2)cc1 ; c1ccc(-c2ccc(N(c3ccc(-c4cccc5ccccc45)cc3)c3ccc4cc(-n5c6ccccc6c6cc7ccccc7cc65)ccc4c3)cc2)cc1]

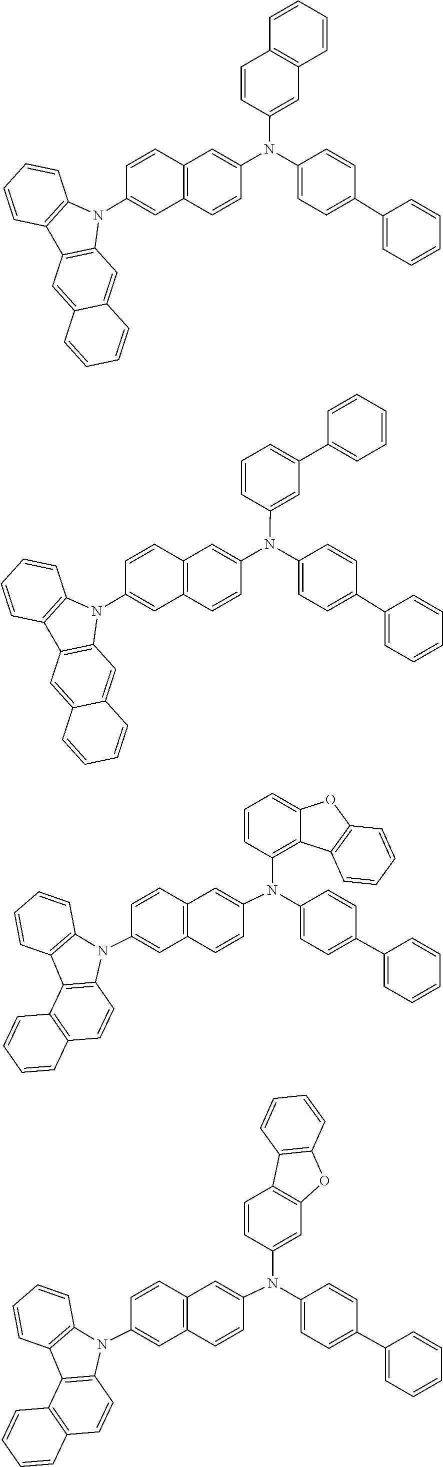 [SMILES: c1ccc(-c2ccc(N(c3ccc4ccccc4c3)c3ccc4cc(-n5c6ccccc6c6cc7ccccc7cc65)ccc4c3)cc2)cc1 ; c1ccc(-c2ccc(N(c3cccc(-c4ccccc4)c3)c3ccc4cc(-n5c6ccccc6c6cc7ccccc7cc65)ccc4c3)cc2)cc1 ; c1ccc(-c2ccc(N(c3ccc4cc(-n5c6ccccc6c6c7ccccc7ccc65)ccc4c3)c3cccc4oc5ccccc5c34)cc2)cc1 ; c1ccc(-c2ccc(N(c3ccc4cc(-n5c6ccccc6c6c7ccccc7ccc65)ccc4c3)c3ccc4c(c3)oc3ccccc34)cc2)cc1]

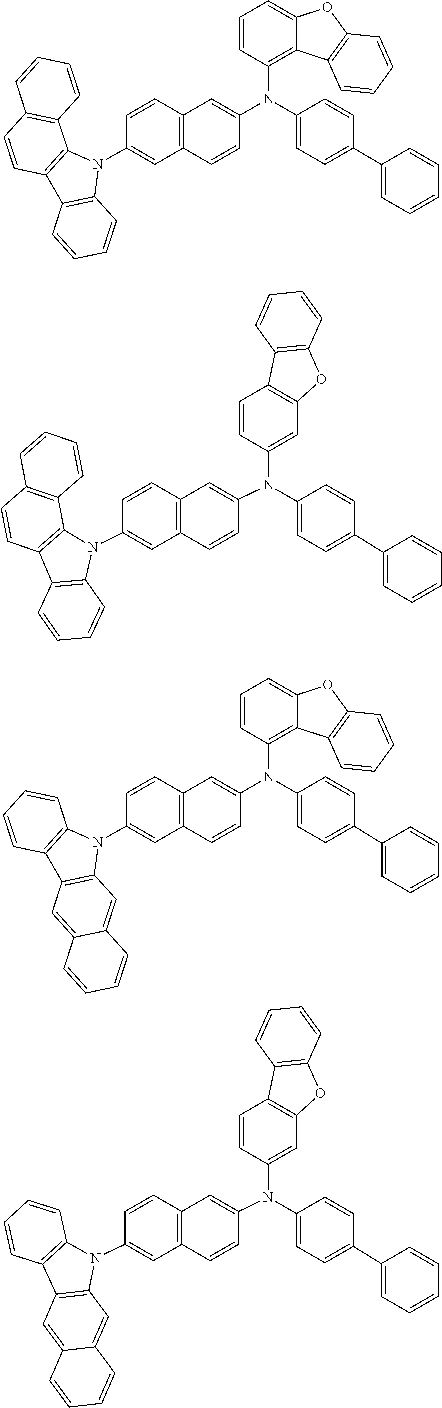 [SMILES: c1ccc(-c2ccc(N(c3ccc4cc(-n5c6ccccc6c6ccc7ccccc7c65)ccc4c3)c3cccc4oc5ccccc5c34)cc2)cc1 ; c1ccc(-c2ccc(N(c3ccc4cc(-n5c6ccccc6c6ccc7ccccc7c65)ccc4c3)c3ccc4c(c3)oc3ccccc34)cc2)cc1 ; c1ccc(-c2ccc(N(c3ccc4cc(-n5c6ccccc6c6cc7ccccc7cc65)ccc4c3)c3cccc4oc5ccccc5c34)cc2)cc1 ; c1ccc(-c2ccc(N(c3ccc4cc(-n5c6ccccc6c6cc7ccccc7cc65)ccc4c3)c3ccc4c(c3)oc3ccccc34)cc2)cc1]

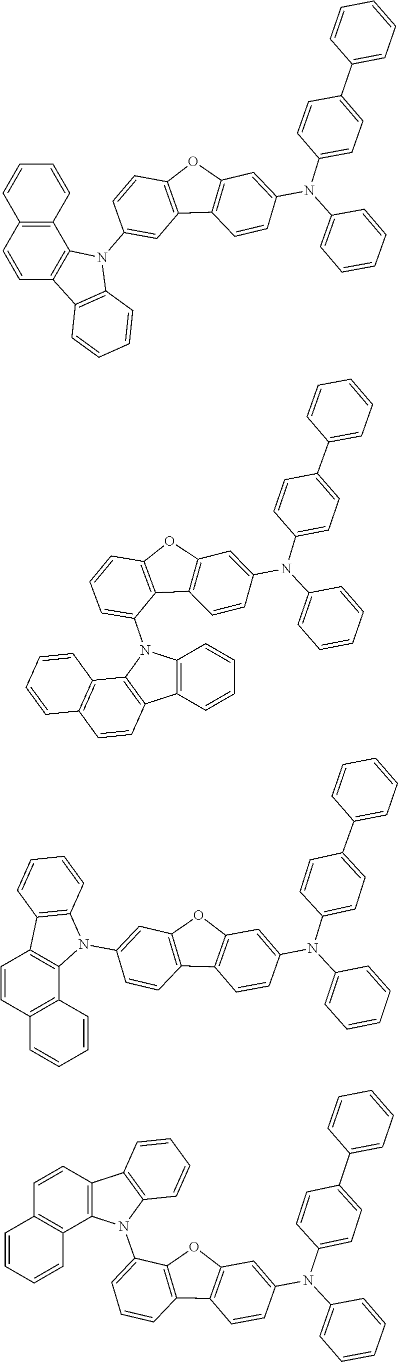 [SMILES: c1ccc(-c2ccc(N(c3ccccc3)c3ccc4c(c3)oc3ccc(-n5c6ccccc6c6ccc7ccccc7c65)cc34)cc2)cc1 ; c1ccc(-c2ccc(N(c3ccccc3)c3ccc4c(c3)oc3cccc(-n5c6ccccc6c6ccc7ccccc7c65)c34)cc2)cc1 ; c1ccc(-c2ccc(N(c3ccccc3)c3ccc4c(c3)oc3cc(-n5c6ccccc6c6ccc7ccccc7c65)ccc34)cc2)cc1 ; c1ccc(-c2ccc(N(c3ccccc3)c3ccc4c(c3)oc3c(-n5c6ccccc6c6ccc7ccccc7c65)cccc34)cc2)cc1]

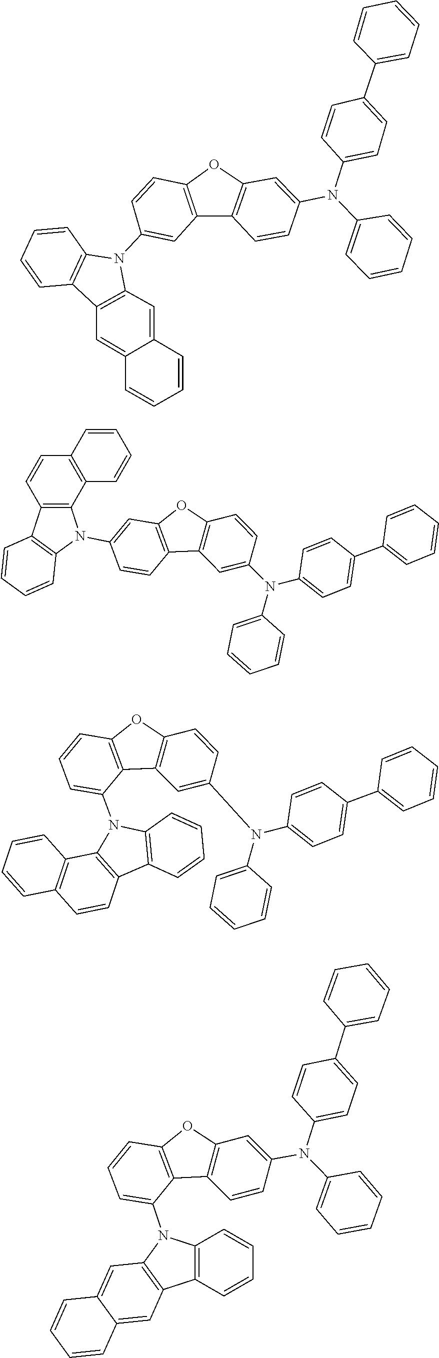

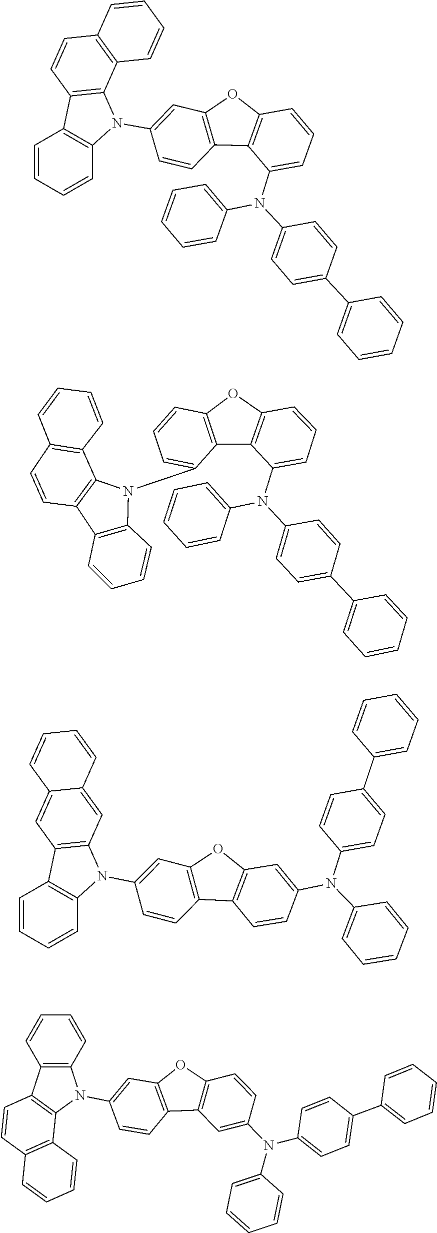

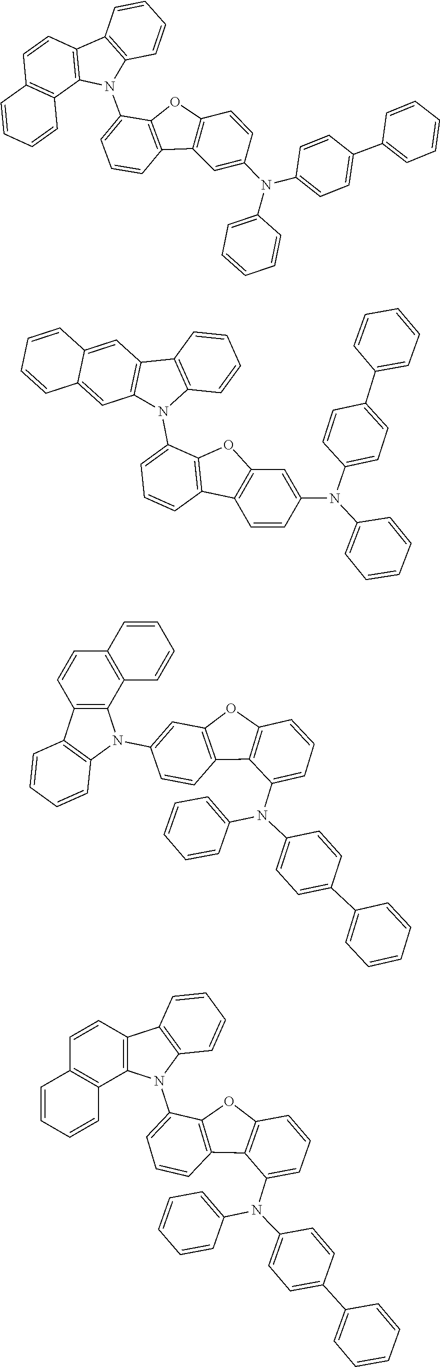 [SMILES: c1ccc(-c2ccc(N(c3ccccc3)c3ccc4oc5c(-n6c7ccccc7c7ccc8ccccc8c76)cccc5c4c3)cc2)cc1 ; c1ccc(-c2ccc(N(c3ccccc3)c3ccc4c(c3)oc3c(-n5c6ccccc6c6cc7ccccc7cc65)cccc34)cc2)cc1 ; c1ccc(-c2ccc(N(c3ccccc3)c3cccc4oc5cc(-n6c7ccccc7c7ccc8ccccc8c76)ccc5c34)cc2)cc1 ; c1ccc(-c2ccc(N(c3ccccc3)c3cccc4oc5c(-n6c7ccccc7c7ccc8ccccc8c76)cccc5c34)cc2)cc1]

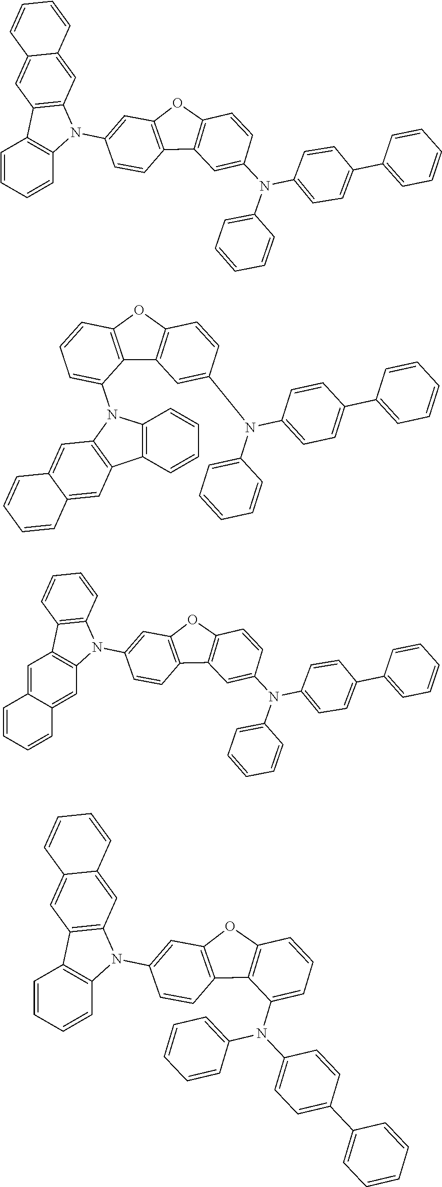 [SMILES: c1ccc(-c2ccc(N(c3ccccc3)c3ccc4oc5cc(-n6c7ccccc7c7cc8ccccc8cc76)ccc5c4c3)cc2)cc1 ; c1ccc(-c2ccc(N(c3ccccc3)c3ccc4oc5cccc(-n6c7ccccc7c7cc8ccccc8cc76)c5c4c3)cc2)cc1 ; c1ccc(-c2ccc(N(c3ccccc3)c3ccc4oc5cc(-n6c7ccccc7c7cc8ccccc8cc76)ccc5c4c3)cc2)cc1 ; c1ccc(-c2ccc(N(c3ccccc3)c3cccc4oc5cc(-n6c7ccccc7c7cc8ccccc8cc76)ccc5c34)cc2)cc1]

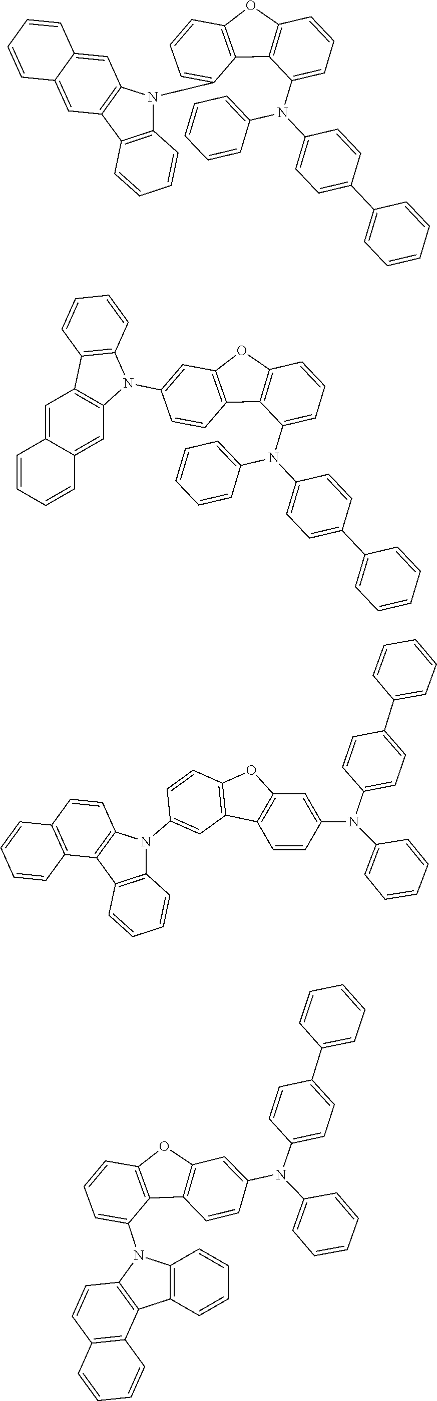

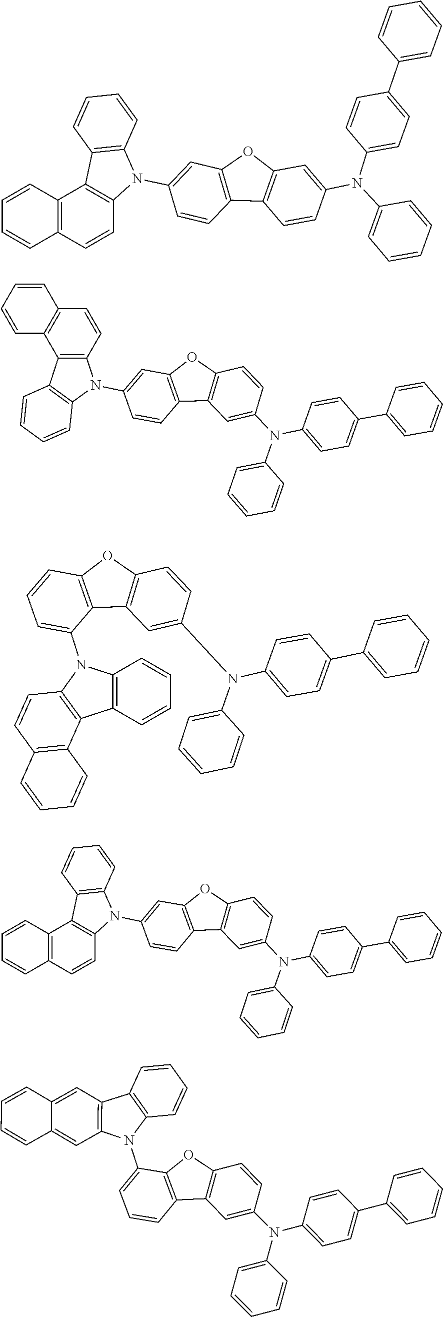

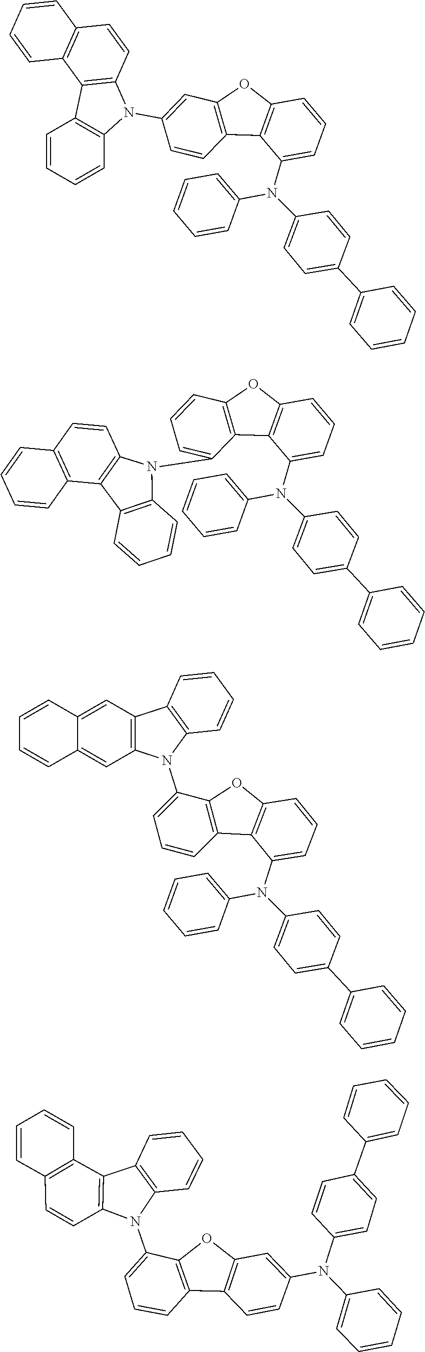

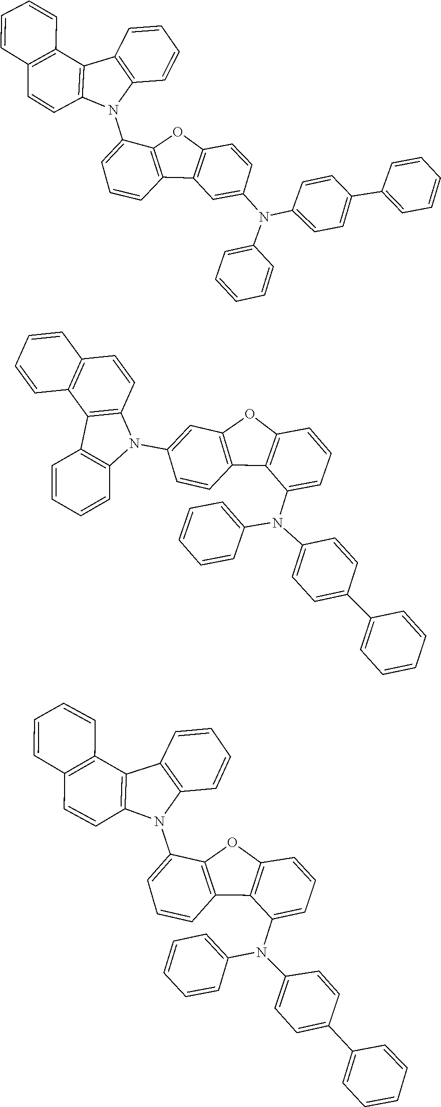 [SMILES: c1ccc(-c2ccc(N(c3ccccc3)c3ccc4oc5c(-n6c7ccccc7c7c8ccccc8ccc76)cccc5c4c3)cc2)cc1 ; c1ccc(-c2ccc(N(c3ccccc3)c3cccc4oc5cc(-n6c7ccccc7c7c8ccccc8ccc76)ccc5c34)cc2)cc1 ; c1ccc(-c2ccc(N(c3ccccc3)c3cccc4oc5c(-n6c7ccccc7c7c8ccccc8ccc76)cccc5c34)cc2)cc1]

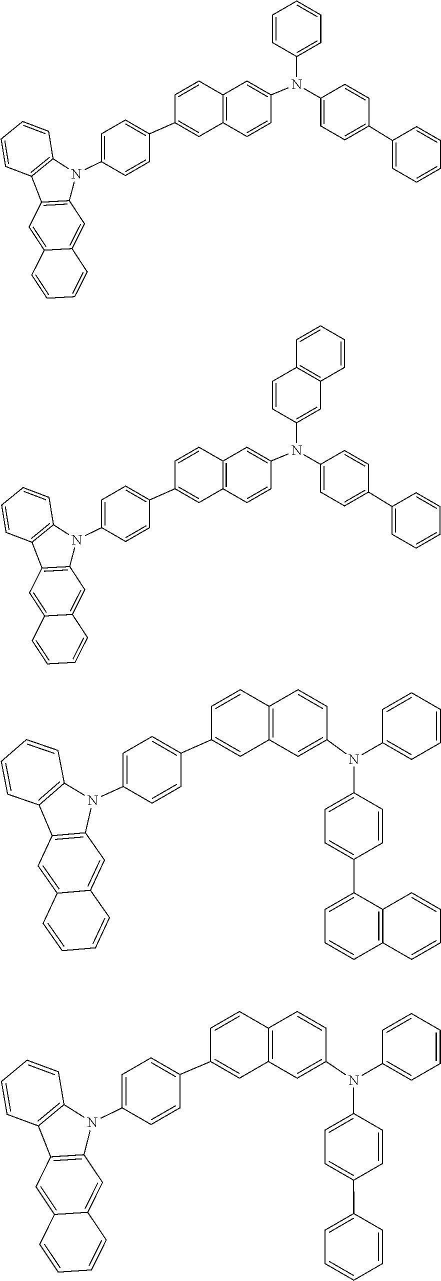 [SMILES: c1ccc(-c2ccc(N(c3ccccc3)c3ccc4cc(-c5ccc(-n6c7ccccc7c7cc8ccccc8cc76)cc5)ccc4c3)cc2)cc1 ; c1ccc(-c2ccc(N(c3ccc4ccccc4c3)c3ccc4cc(-c5ccc(-n6c7ccccc7c7cc8ccccc8cc76)cc5)ccc4c3)cc2)cc1 ; c1ccc(N(c2ccc(-c3cccc4ccccc34)cc2)c2ccc3ccc(-c4ccc(-n5c6ccccc6c6cc7ccccc7cc65)cc4)cc3c2)cc1 ; c1ccc(-c2ccc(N(c3ccccc3)c3ccc4ccc(-c5ccc(-n6c7ccccc7c7cc8ccccc8cc76)cc5)cc4c3)cc2)cc1]

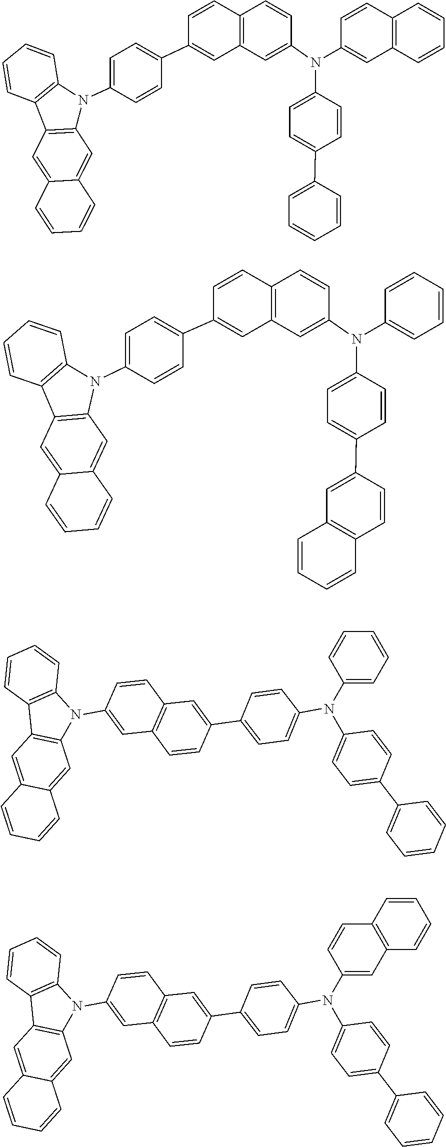

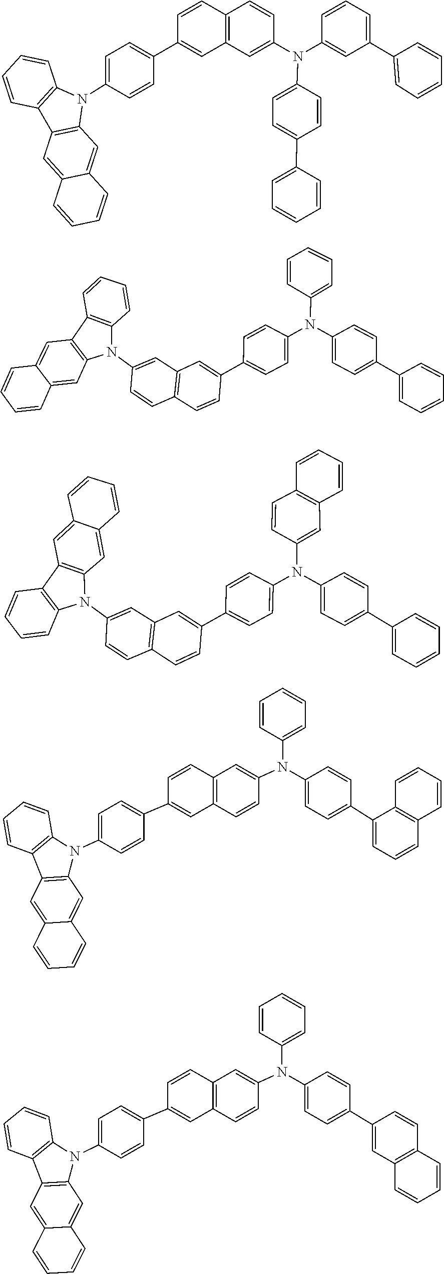

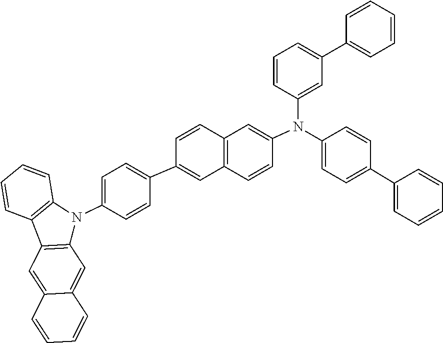 [SMILES: c1ccc(-c2ccc(N(c3cccc(-c4ccccc4)c3)c3ccc4cc(-c5ccc(-n6c7ccccc7c7cc8ccccc8cc76)cc5)ccc4c3)cc2)cc1]

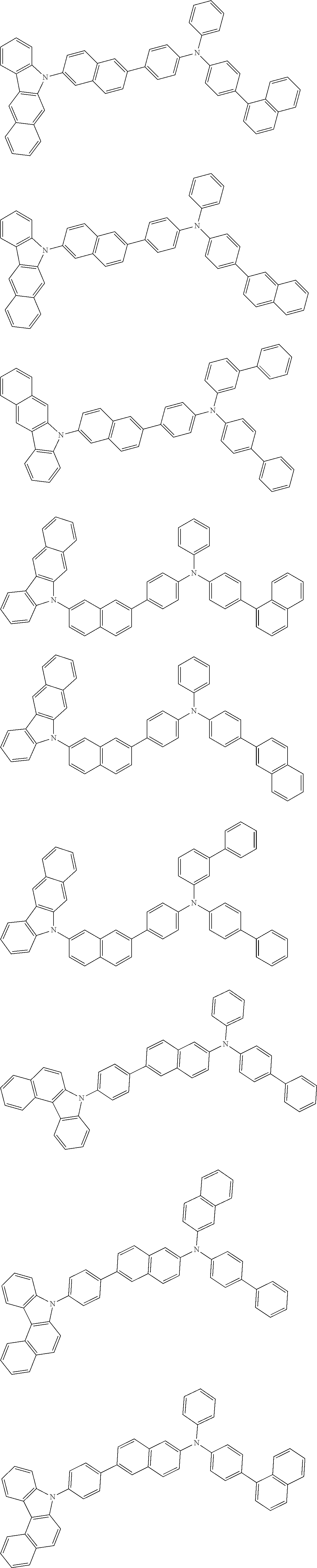

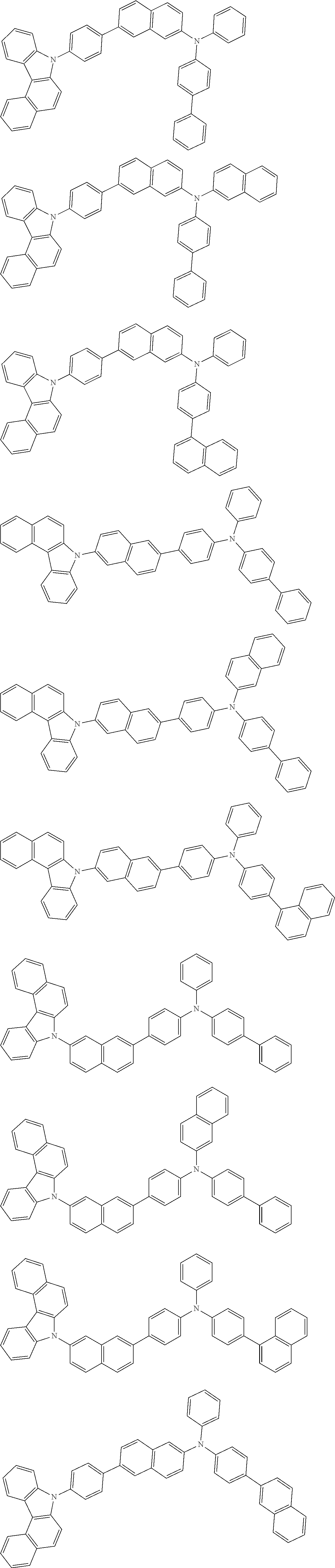

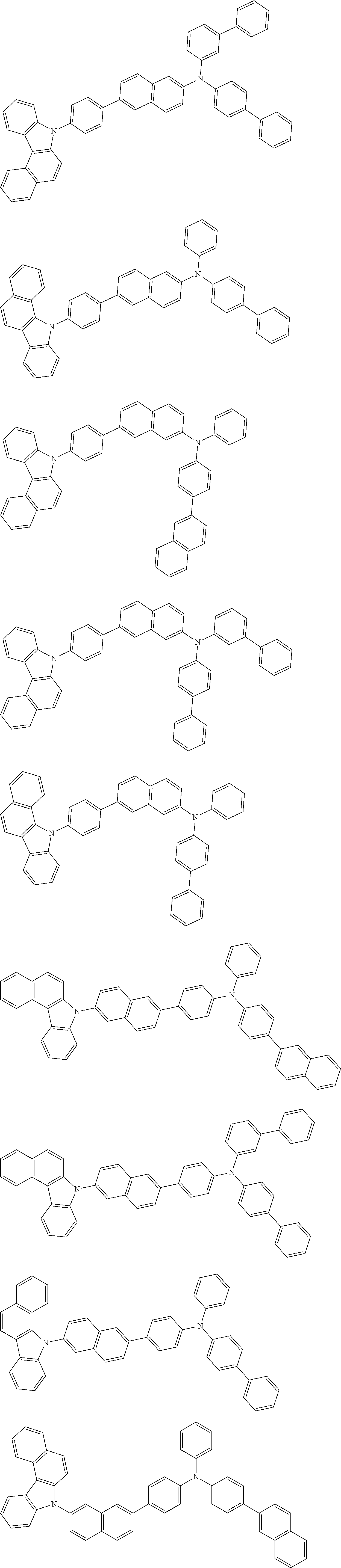

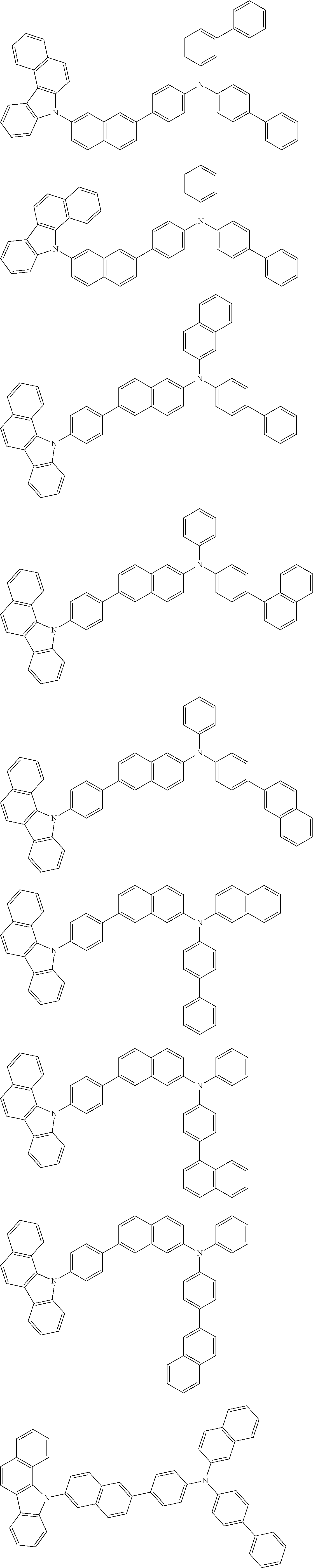

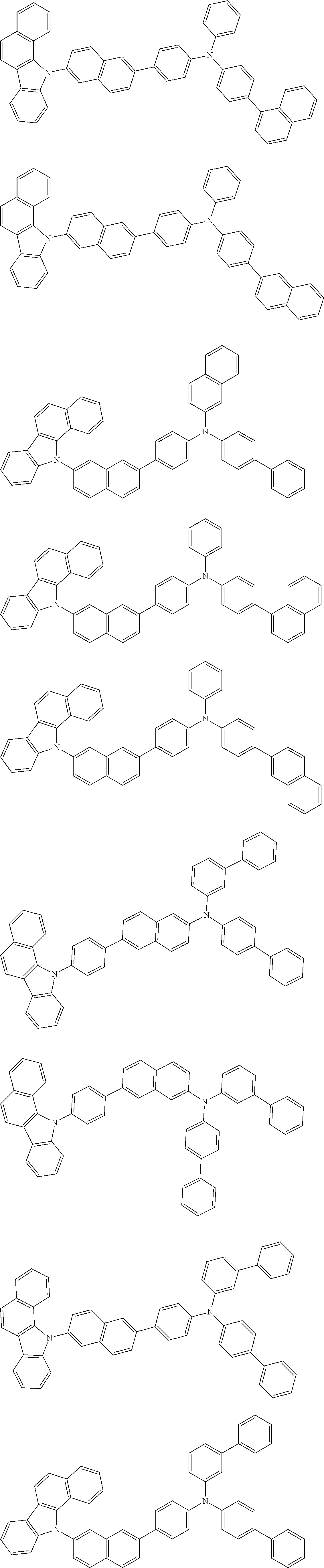

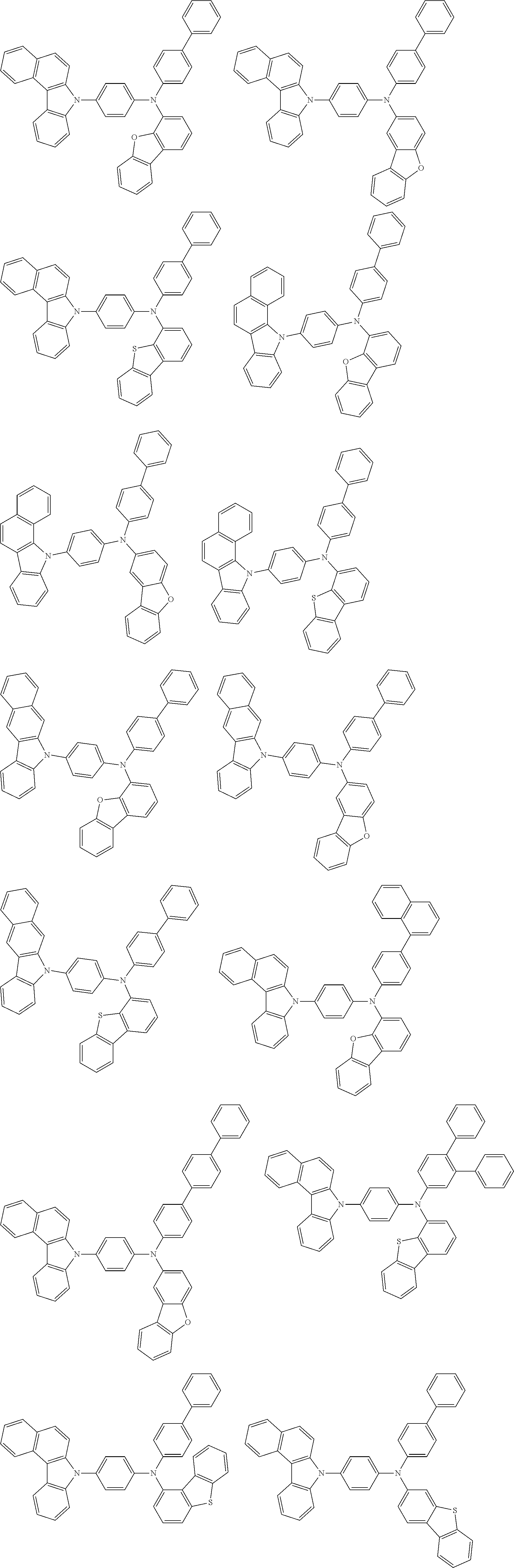

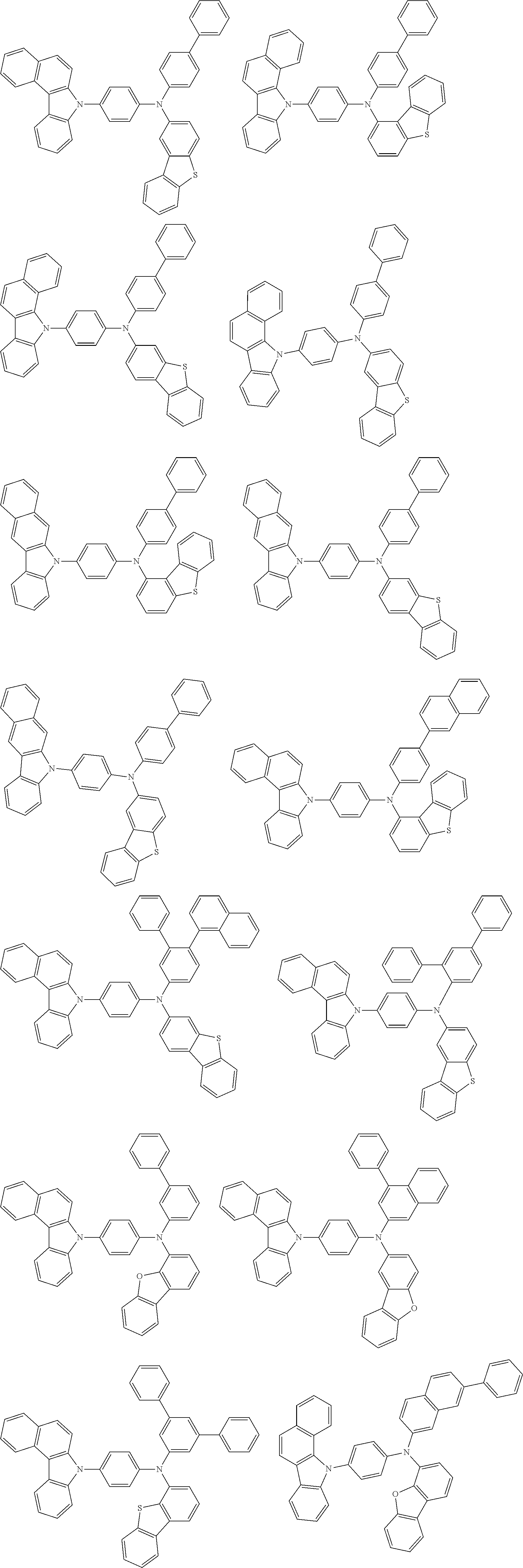

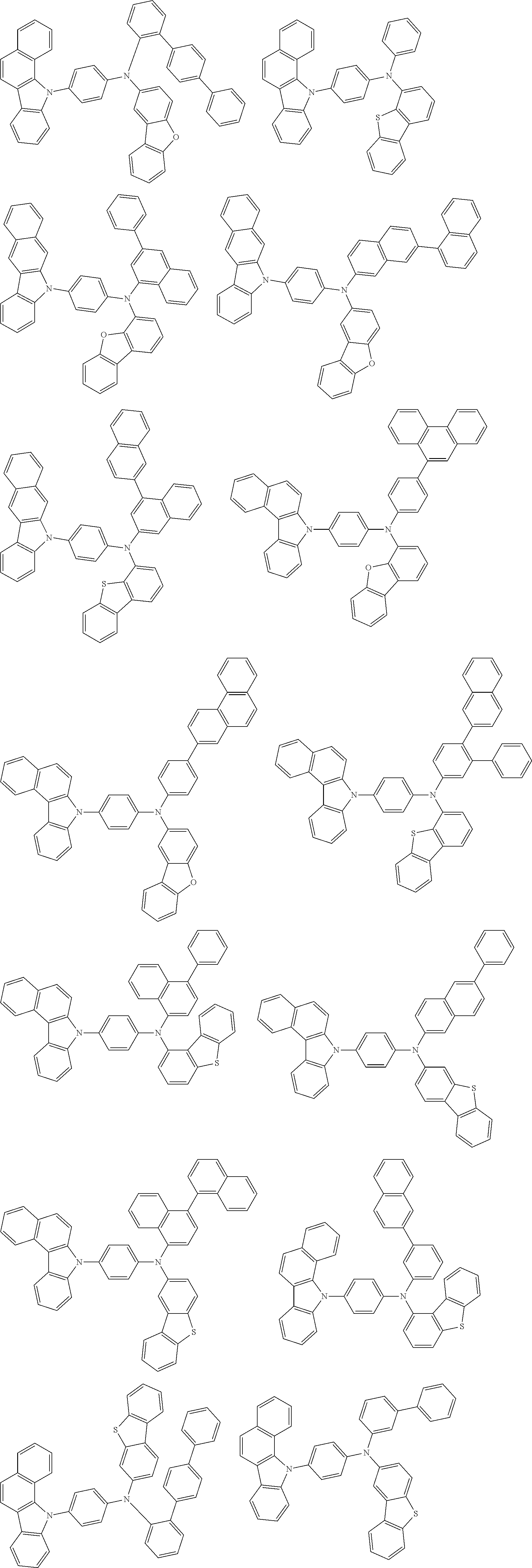

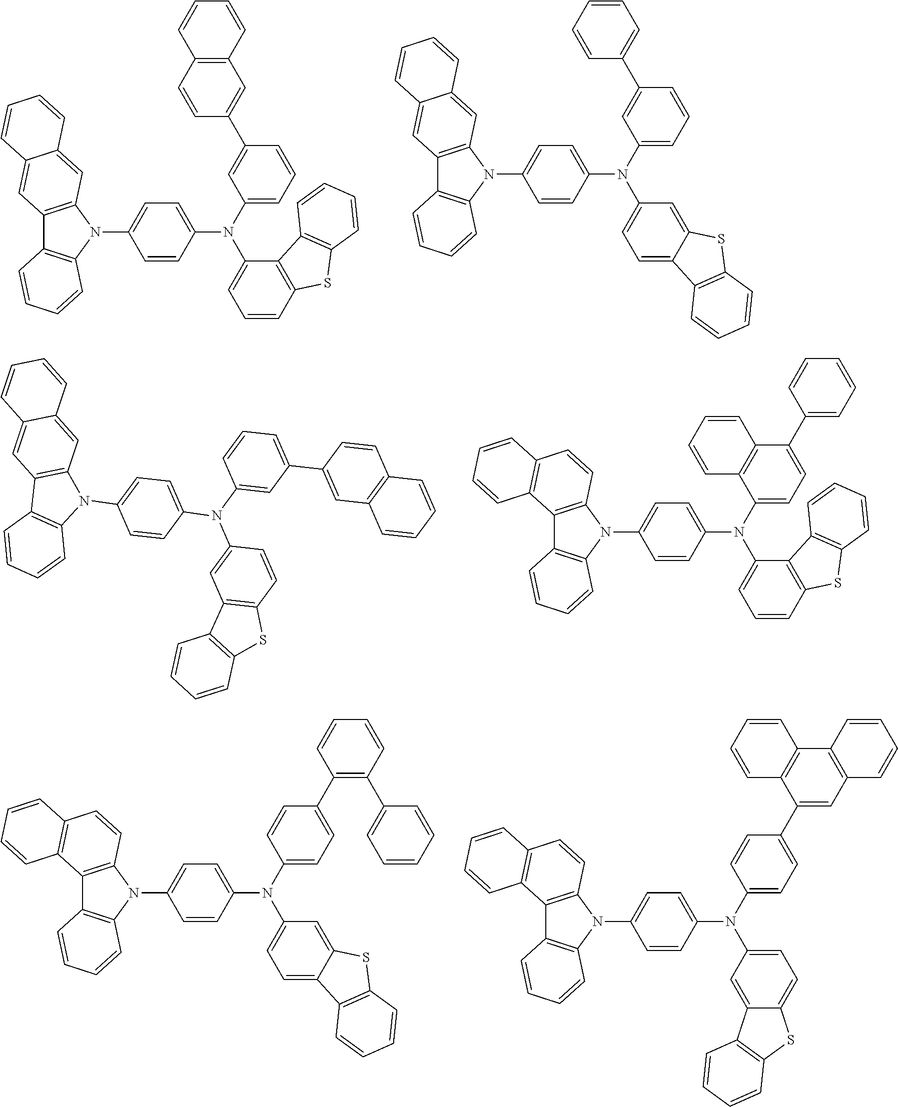

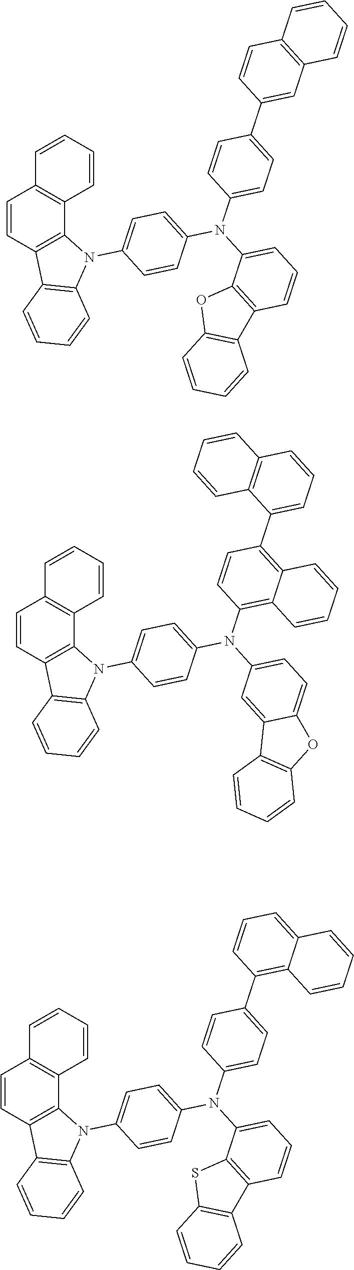

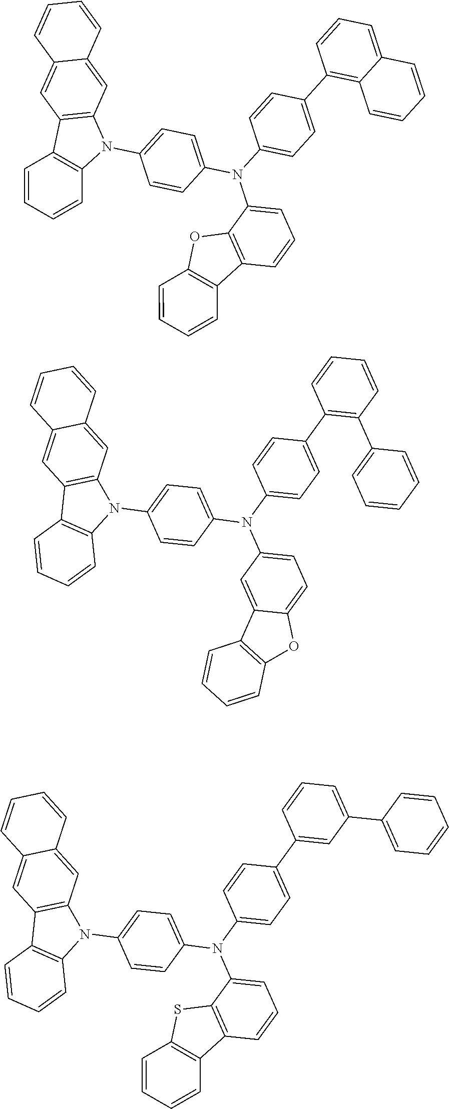 [SMILES: c1ccc2cc3c(cc2c1)c1ccccc1n3-c1ccc(N(c2ccc(-c3cccc4ccccc34)cc2)c2cccc3c2oc2ccccc23)cc1 ; c1ccc(-c2ccccc2-c2ccc(N(c3ccc(-n4c5ccccc5c5cc6ccccc6cc54)cc3)c3ccc4oc5ccccc5c4c3)cc2)cc1 ; c1ccc(-c2cccc(-c3ccc(N(c4ccc(-n5c6ccccc6c6cc7ccccc7cc65)cc4)c4cccc5c4sc4ccccc45)cc3)c2)cc1]

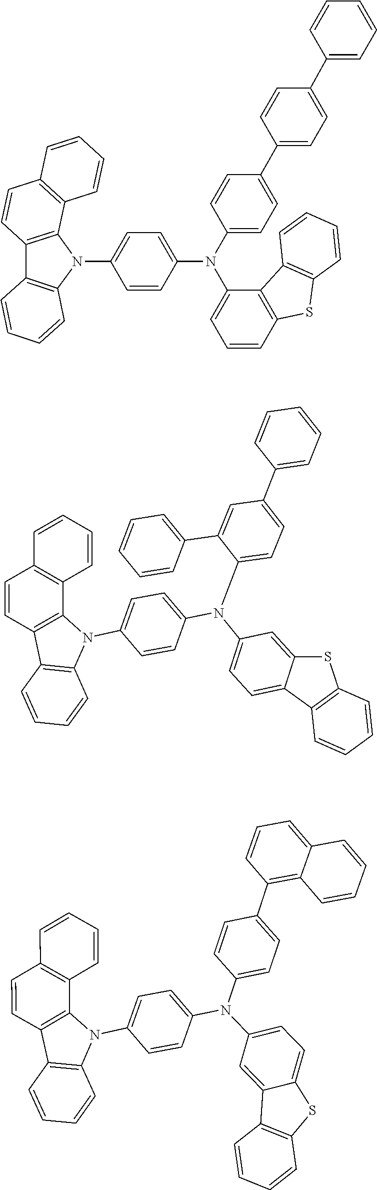 [SMILES: c1ccc(-c2ccc(-c3ccc(N(c4ccc(-n5c6ccccc6c6ccc7ccccc7c65)cc4)c4cccc5sc6ccccc6c45)cc3)cc2)cc1 ; c1ccc(-c2ccc(N(c3ccc(-n4c5ccccc5c5ccc6ccccc6c54)cc3)c3ccc4c(c3)sc3ccccc34)c(-c3ccccc3)c2)cc1 ; c1ccc2c(-c3ccc(N(c4ccc(-n5c6ccccc6c6ccc7ccccc7c65)cc4)c4ccc5sc6ccccc6c5c4)cc3)cccc2c1]

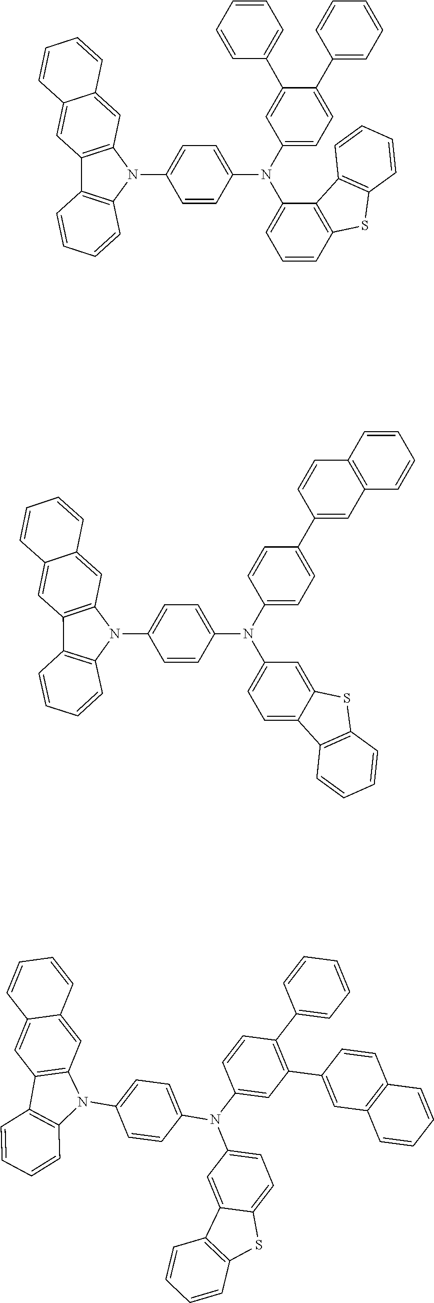 [SMILES: c1ccc(-c2ccc(N(c3ccc(-n4c5ccccc5c5cc6ccccc6cc54)cc3)c3cccc4sc5ccccc5c34)cc2-c2ccccc2)cc1 ; c1ccc2cc(-c3ccc(N(c4ccc(-n5c6ccccc6c6cc7ccccc7cc65)cc4)c4ccc5c(c4)sc4ccccc45)cc3)ccc2c1 ; c1ccc(-c2ccc(N(c3ccc(-n4c5ccccc5c5cc6ccccc6cc54)cc3)c3ccc4sc5ccccc5c4c3)cc2-c2ccc3ccccc3c2)cc1]

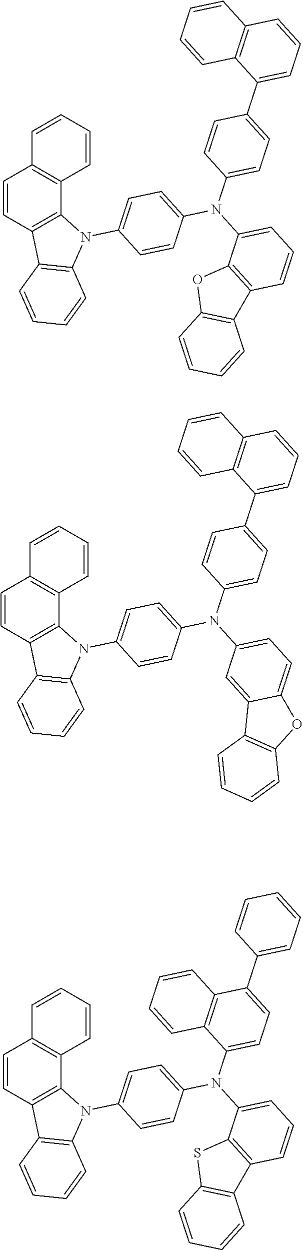

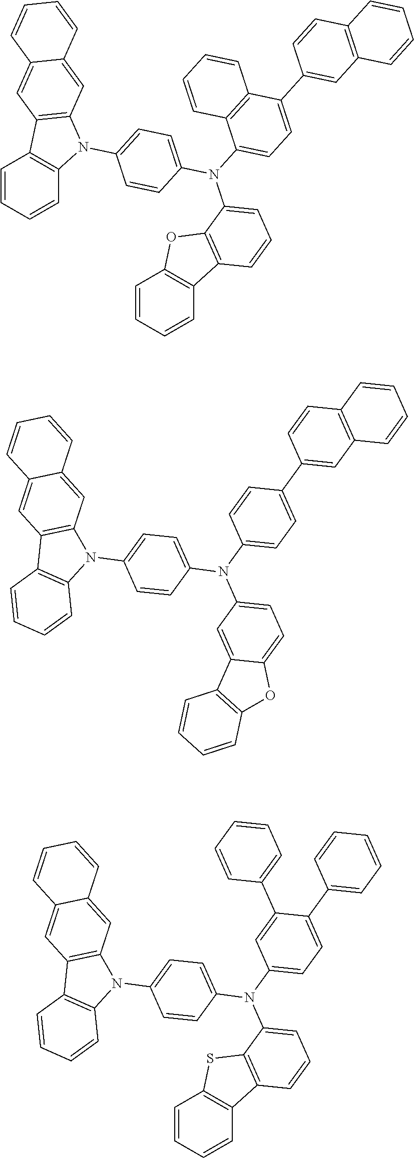 [SMILES: c1ccc2cc(-c3ccc(N(c4ccc(-n5c6ccccc6c6cc7ccccc7cc65)cc4)c4cccc5c4oc4ccccc45)c4ccccc34)ccc2c1 ; c1ccc2cc(-c3ccc(N(c4ccc(-n5c6ccccc6c6cc7ccccc7cc65)cc4)c4ccc5oc6ccccc6c5c4)cc3)ccc2c1 ; c1ccc(-c2ccc(N(c3ccc(-n4c5ccccc5c5cc6ccccc6cc54)cc3)c3cccc4c3sc3ccccc34)cc2-c2ccccc2)cc1]

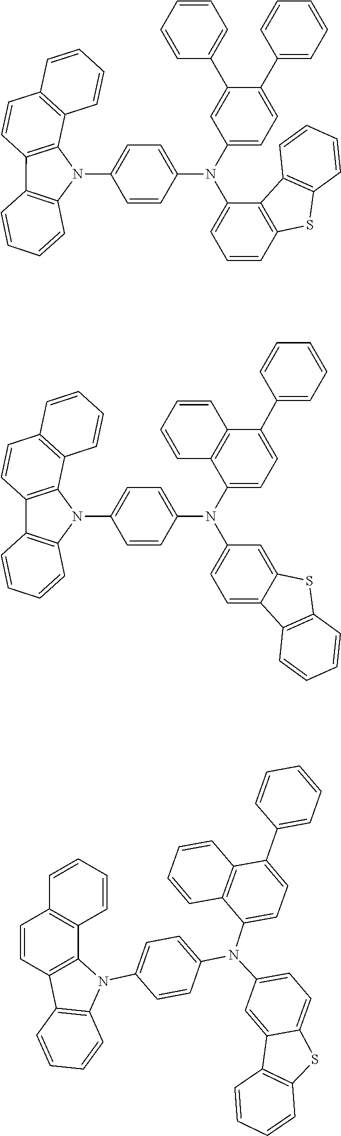 [SMILES: c1ccc(-c2ccc(N(c3ccc(-n4c5ccccc5c5ccc6ccccc6c54)cc3)c3cccc4sc5ccccc5c34)cc2-c2ccccc2)cc1 ; c1ccc(-c2ccc(N(c3ccc(-n4c5ccccc5c5ccc6ccccc6c54)cc3)c3ccc4c(c3)sc3ccccc34)c3ccccc23)cc1 ; c1ccc(-c2ccc(N(c3ccc(-n4c5ccccc5c5ccc6ccccc6c54)cc3)c3ccc4sc5ccccc5c4c3)c3ccccc23)cc1]

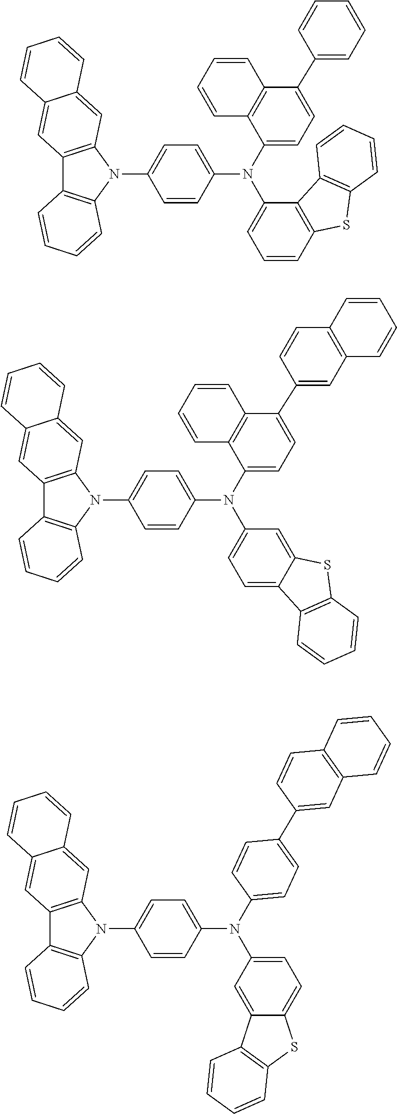 [SMILES: c1ccc(-c2ccc(N(c3ccc(-n4c5ccccc5c5cc6ccccc6cc54)cc3)c3cccc4sc5ccccc5c34)c3ccccc23)cc1 ; c1ccc2cc(-c3ccc(N(c4ccc(-n5c6ccccc6c6cc7ccccc7cc65)cc4)c4ccc5c(c4)sc4ccccc45)c4ccccc34)ccc2c1 ; c1ccc2cc(-c3ccc(N(c4ccc(-n5c6ccccc6c6cc7ccccc7cc65)cc4)c4ccc5sc6ccccc6c5c4)cc3)ccc2c1]

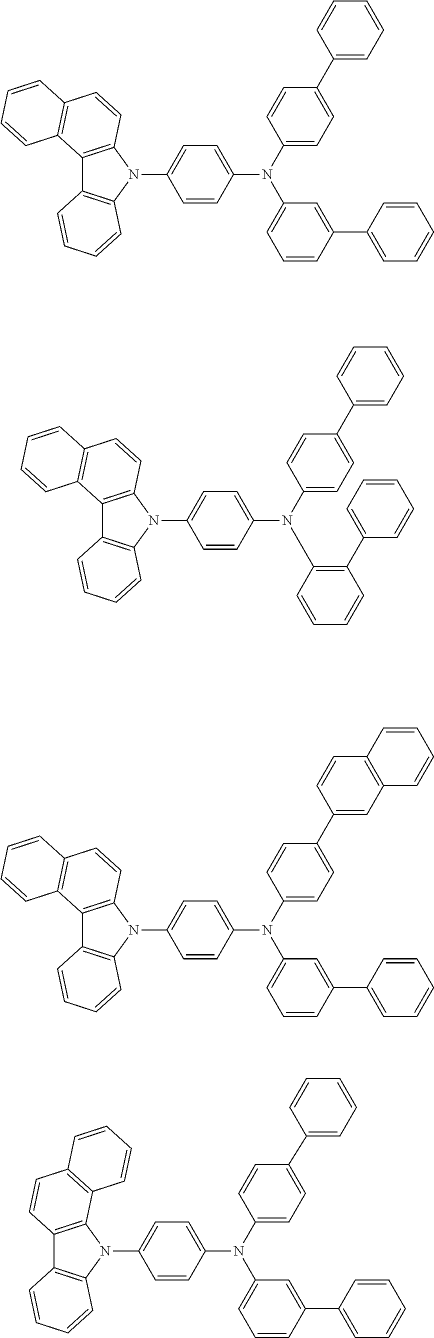 [SMILES: c1ccc(-c2ccc(N(c3ccc(-n4c5ccccc5c5c6ccccc6ccc54)cc3)c3cccc(-c4ccccc4)c3)cc2)cc1 ; c1ccc(-c2ccc(N(c3ccc(-n4c5ccccc5c5c6ccccc6ccc54)cc3)c3ccccc3-c3ccccc3)cc2)cc1 ; c1ccc(-c2cccc(N(c3ccc(-c4ccc5ccccc5c4)cc3)c3ccc(-n4c5ccccc5c5c6ccccc6ccc54)cc3)c2)cc1 ; c1ccc(-c2ccc(N(c3ccc(-n4c5ccccc5c5ccc6ccccc6c54)cc3)c3cccc(-c4ccccc4)c3)cc2)cc1]

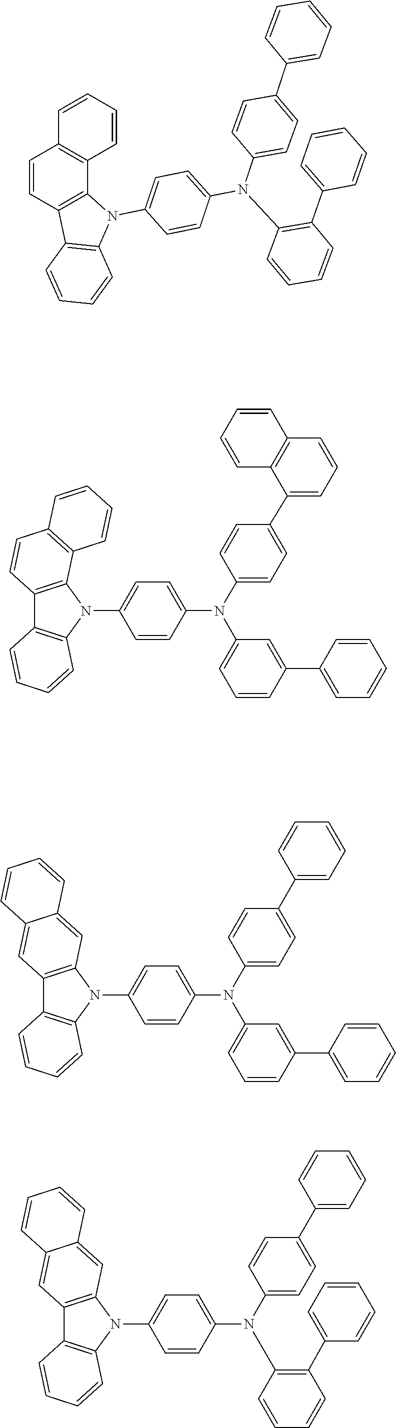 [SMILES: c1ccc(-c2ccc(N(c3ccc(-n4c5ccccc5c5ccc6ccccc6c54)cc3)c3ccccc3-c3ccccc3)cc2)cc1 ; c1ccc(-c2cccc(N(c3ccc(-c4cccc5ccccc45)cc3)c3ccc(-n4c5ccccc5c5ccc6ccccc6c54)cc3)c2)cc1 ; c1ccc(-c2ccc(N(c3ccc(-n4c5ccccc5c5cc6ccccc6cc54)cc3)c3cccc(-c4ccccc4)c3)cc2)cc1 ; c1ccc(-c2ccc(N(c3ccc(-n4c5ccccc5c5cc6ccccc6cc54)cc3)c3ccccc3-c3ccccc3)cc2)cc1]

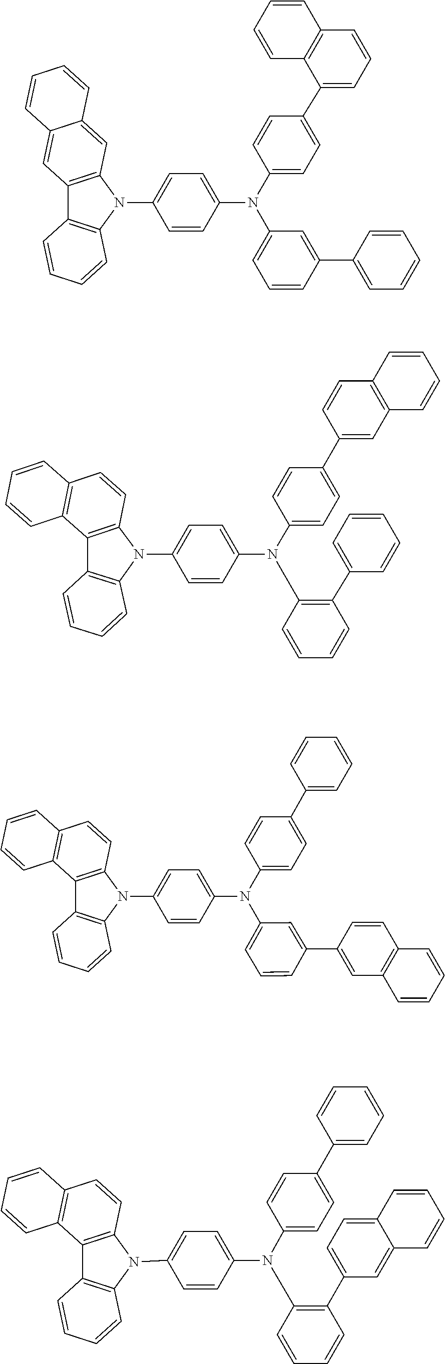 [SMILES: c1ccc(-c2cccc(N(c3ccc(-c4cccc5ccccc45)cc3)c3ccc(-n4c5ccccc5c5cc6ccccc6cc54)cc3)c2)cc1 ; c1ccc(-c2ccccc2N(c2ccc(-c3ccc4ccccc4c3)cc2)c2ccc(-n3c4ccccc4c4c5ccccc5ccc43)cc2)cc1 ; c1ccc(-c2ccc(N(c3ccc(-n4c5ccccc5c5c6ccccc6ccc54)cc3)c3cccc(-c4ccc5ccccc5c4)c3)cc2)cc1 ; c1ccc(-c2ccc(N(c3ccc(-n4c5ccccc5c5c6ccccc6ccc54)cc3)c3ccccc3-c3ccc4ccccc4c3)cc2)cc1]

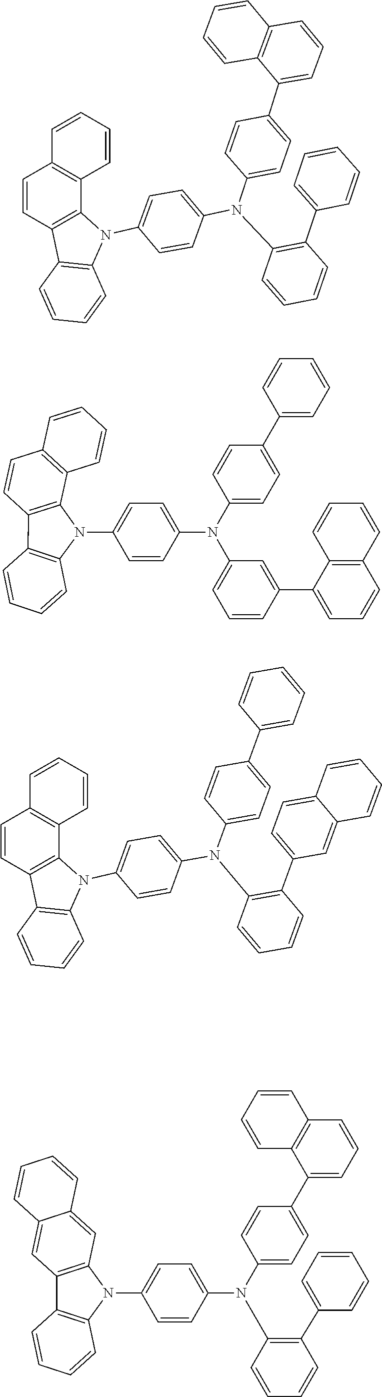 [SMILES: c1ccc(-c2ccccc2N(c2ccc(-c3cccc4ccccc34)cc2)c2ccc(-n3c4ccccc4c4ccc5ccccc5c43)cc2)cc1 ; c1ccc(-c2ccc(N(c3ccc(-n4c5ccccc5c5ccc6ccccc6c54)cc3)c3cccc(-c4cccc5ccccc45)c3)cc2)cc1 ; c1ccc(-c2ccc(N(c3ccc(-n4c5ccccc5c5ccc6ccccc6c54)cc3)c3ccccc3-c3ccc4ccccc4c3)cc2)cc1 ; c1ccc(-c2ccccc2N(c2ccc(-c3cccc4ccccc34)cc2)c2ccc(-n3c4ccccc4c4cc5ccccc5cc43)cc2)cc1]

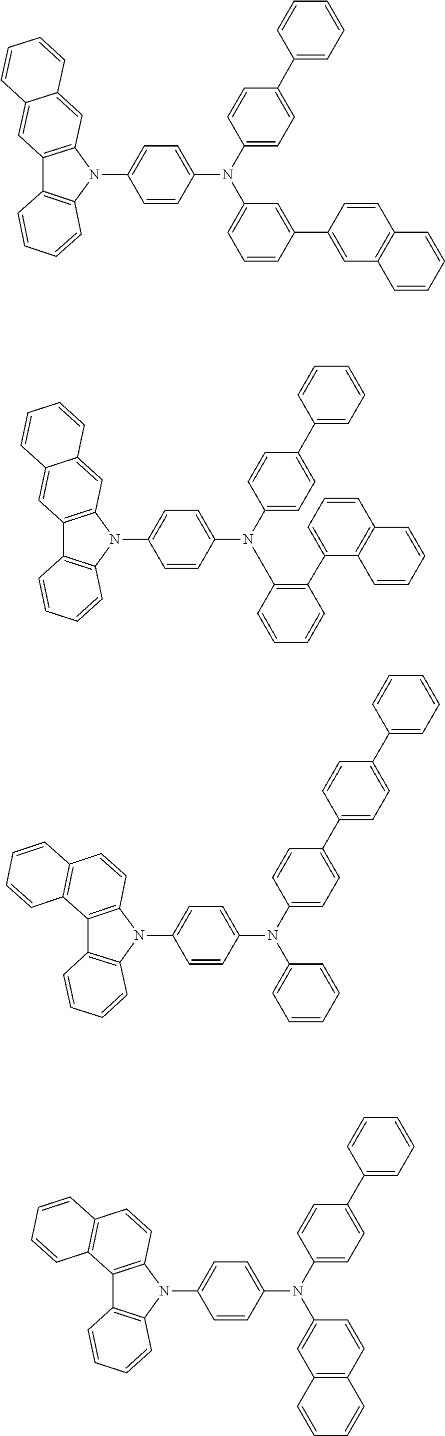 [SMILES: c1ccc(-c2ccc(N(c3ccc(-n4c5ccccc5c5cc6ccccc6cc54)cc3)c3cccc(-c4ccc5ccccc5c4)c3)cc2)cc1 ; c1ccc(-c2ccc(N(c3ccc(-n4c5ccccc5c5cc6ccccc6cc54)cc3)c3ccccc3-c3cccc4ccccc34)cc2)cc1 ; c1ccc(-c2ccc(-c3ccc(N(c4ccccc4)c4ccc(-n5c6ccccc6c6c7ccccc7ccc65)cc4)cc3)cc2)cc1 ; c1ccc(-c2ccc(N(c3ccc(-n4c5ccccc5c5c6ccccc6ccc54)cc3)c3ccc4ccccc4c3)cc2)cc1]

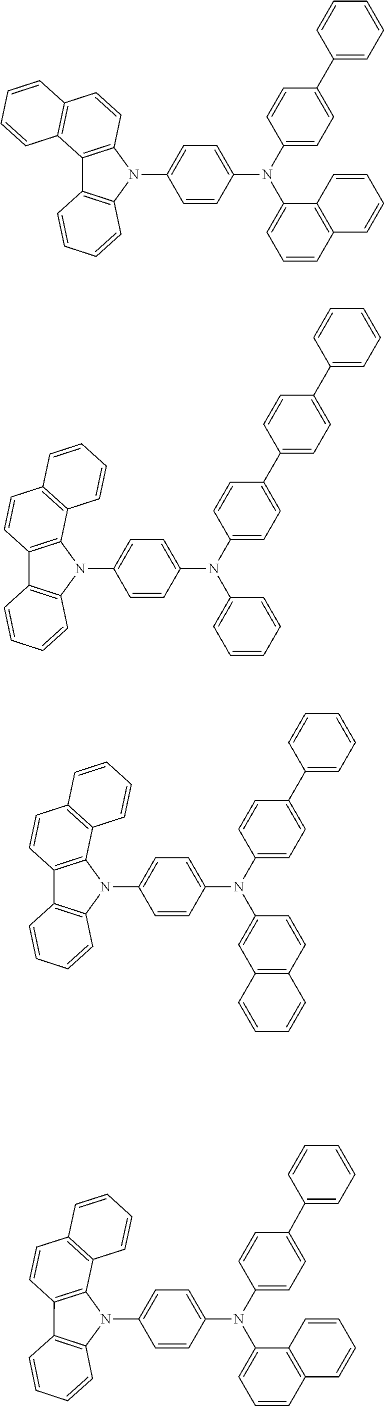 [SMILES: c1ccc(-c2ccc(N(c3ccc(-n4c5ccccc5c5c6ccccc6ccc54)cc3)c3cccc4ccccc34)cc2)cc1 ; c1ccc(-c2ccc(-c3ccc(N(c4ccccc4)c4ccc(-n5c6ccccc6c6ccc7ccccc7c65)cc4)cc3)cc2)cc1 ; c1ccc(-c2ccc(N(c3ccc(-n4c5ccccc5c5ccc6ccccc6c54)cc3)c3ccc4ccccc4c3)cc2)cc1 ; c1ccc(-c2ccc(N(c3ccc(-n4c5ccccc5c5ccc6ccccc6c54)cc3)c3cccc4ccccc34)cc2)cc1]

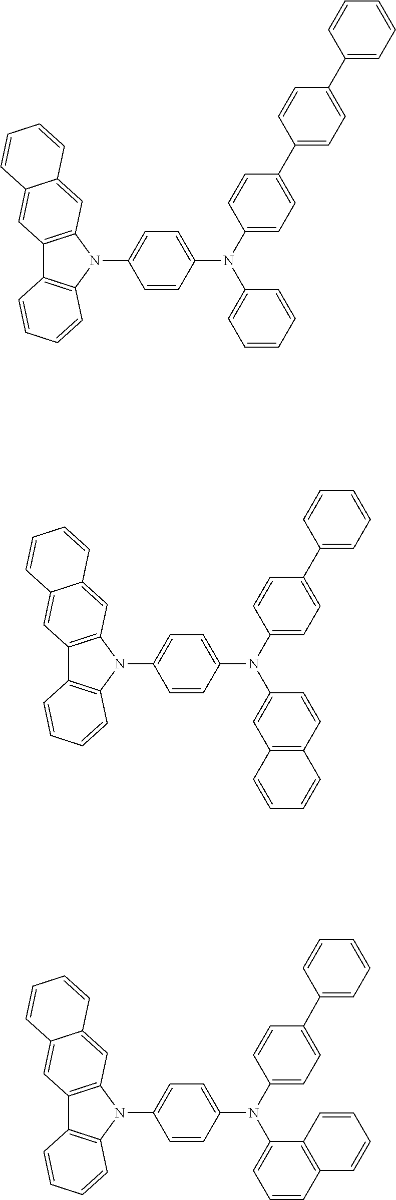 [SMILES: c1ccc(-c2ccc(-c3ccc(N(c4ccccc4)c4ccc(-n5c6ccccc6c6cc7ccccc7cc65)cc4)cc3)cc2)cc1 ; c1ccc(-c2ccc(N(c3ccc(-n4c5ccccc5c5cc6ccccc6cc54)cc3)c3ccc4ccccc4c3)cc2)cc1 ; c1ccc(-c2ccc(N(c3ccc(-n4c5ccccc5c5cc6ccccc6cc54)cc3)c3cccc4ccccc34)cc2)cc1]

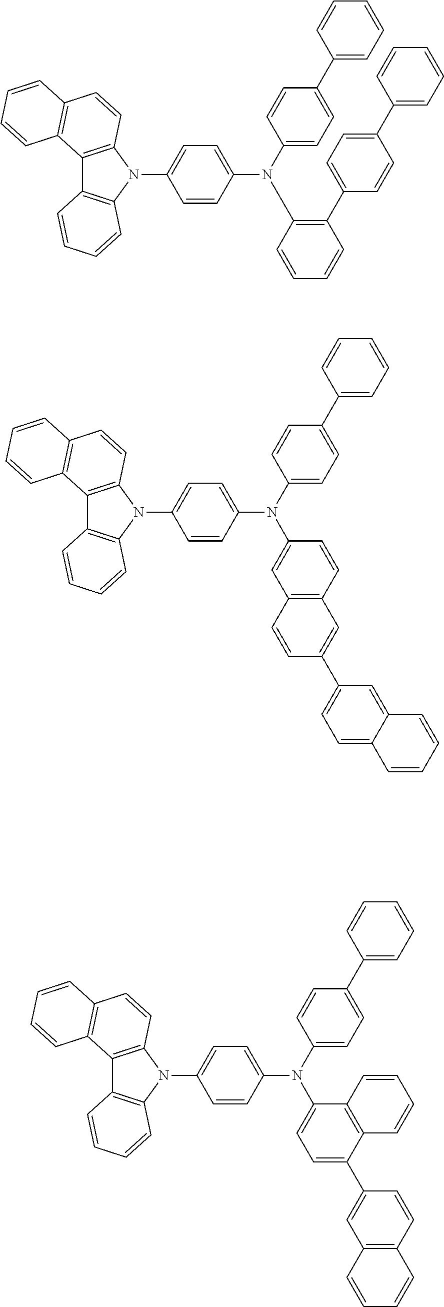 [SMILES: c1ccc(-c2ccc(-c3ccccc3N(c3ccc(-c4ccccc4)cc3)c3ccc(-n4c5ccccc5c5c6ccccc6ccc54)cc3)cc2)cc1 ; c1ccc(-c2ccc(N(c3ccc(-n4c5ccccc5c5c6ccccc6ccc54)cc3)c3ccc4cc(-c5ccc6ccccc6c5)ccc4c3)cc2)cc1 ; c1ccc(-c2ccc(N(c3ccc(-n4c5ccccc5c5c6ccccc6ccc54)cc3)c3ccc(-c4ccc5ccccc5c4)c4ccccc34)cc2)cc1]

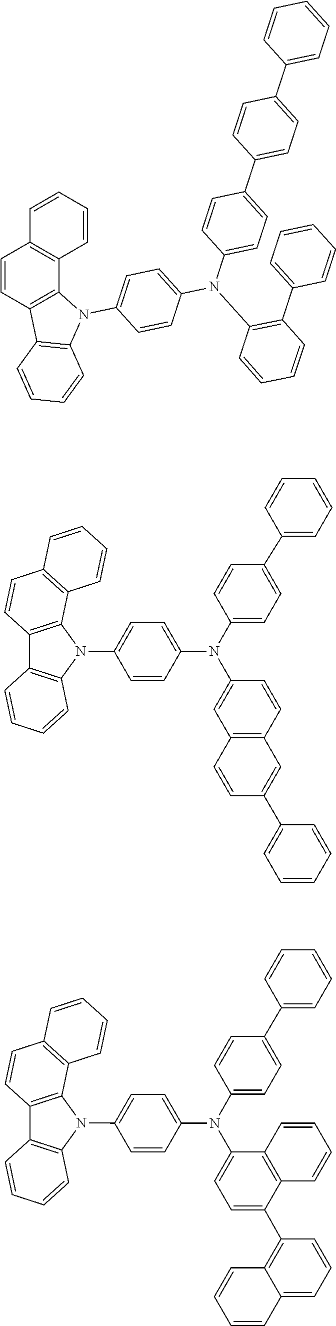 [SMILES: c1ccc(-c2ccc(-c3ccc(N(c4ccc(-n5c6ccccc6c6ccc7ccccc7c65)cc4)c4ccccc4-c4ccccc4)cc3)cc2)cc1 ; c1ccc(-c2ccc(N(c3ccc(-n4c5ccccc5c5ccc6ccccc6c54)cc3)c3ccc4cc(-c5ccccc5)ccc4c3)cc2)cc1 ; c1ccc(-c2ccc(N(c3ccc(-n4c5ccccc5c5ccc6ccccc6c54)cc3)c3ccc(-c4cccc5ccccc45)c4ccccc34)cc2)cc1]

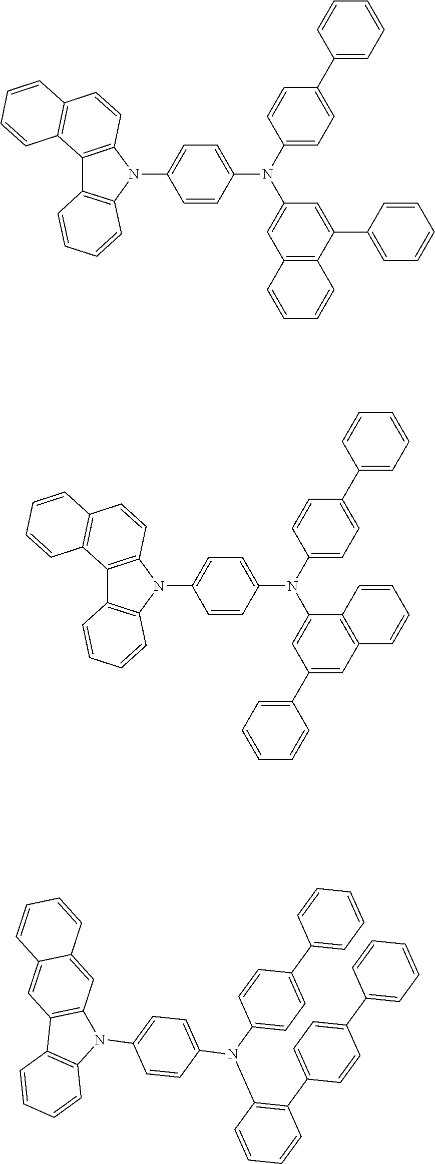 [SMILES: c1ccc(-c2ccc(N(c3ccc(-n4c5ccccc5c5c6ccccc6ccc54)cc3)c3cc(-c4ccccc4)c4ccccc4c3)cc2)cc1 ; c1ccc(-c2ccc(N(c3ccc(-n4c5ccccc5c5c6ccccc6ccc54)cc3)c3cc(-c4ccccc4)cc4ccccc34)cc2)cc1 ; c1ccc(-c2ccc(-c3ccccc3N(c3ccc(-c4ccccc4)cc3)c3ccc(-n4c5ccccc5c5cc6ccccc6cc54)cc3)cc2)cc1]

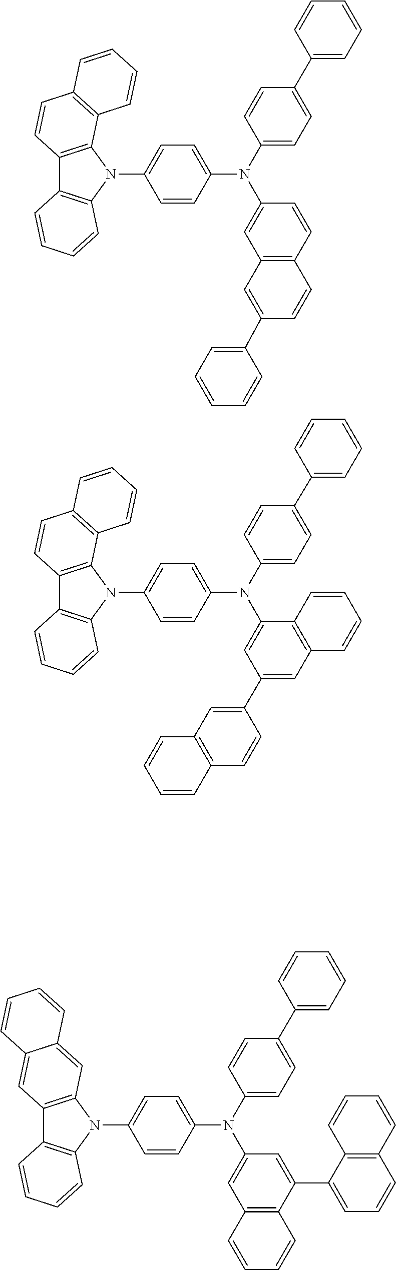 [SMILES: c1ccc(-c2ccc(N(c3ccc(-n4c5ccccc5c5ccc6ccccc6c54)cc3)c3ccc4ccc(-c5ccccc5)cc4c3)cc2)cc1 ; c1ccc(-c2ccc(N(c3ccc(-n4c5ccccc5c5ccc6ccccc6c54)cc3)c3cc(-c4ccc5ccccc5c4)cc4ccccc34)cc2)cc1 ; c1ccc(-c2ccc(N(c3ccc(-n4c5ccccc5c5cc6ccccc6cc54)cc3)c3cc(-c4cccc5ccccc45)c4ccccc4c3)cc2)cc1]

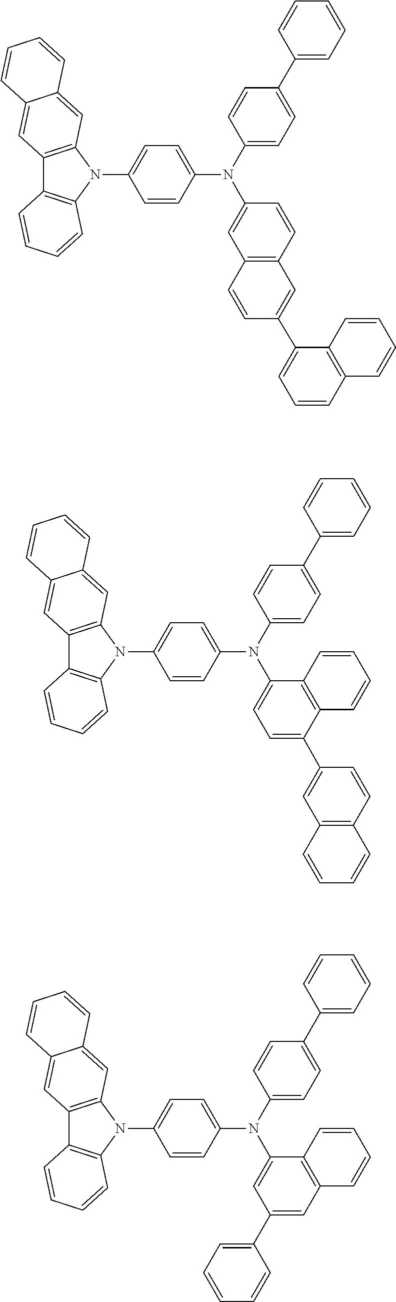 [SMILES: c1ccc(-c2ccc(N(c3ccc(-n4c5ccccc5c5cc6ccccc6cc54)cc3)c3ccc4cc(-c5cccc6ccccc56)ccc4c3)cc2)cc1 ; c1ccc(-c2ccc(N(c3ccc(-n4c5ccccc5c5cc6ccccc6cc54)cc3)c3ccc(-c4ccc5ccccc5c4)c4ccccc34)cc2)cc1 ; c1ccc(-c2ccc(N(c3ccc(-n4c5ccccc5c5cc6ccccc6cc54)cc3)c3cc(-c4ccccc4)cc4ccccc34)cc2)cc1]

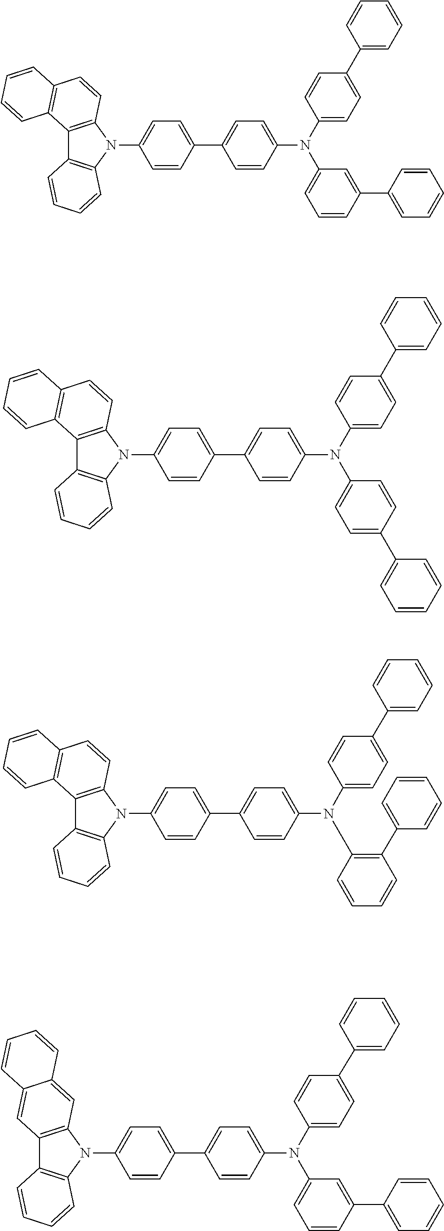 [SMILES: c1ccc(-c2ccc(N(c3ccc(-c4ccc(-n5c6ccccc6c6c7ccccc7ccc65)cc4)cc3)c3cccc(-c4ccccc4)c3)cc2)cc1 ; c1ccc(-c2ccc(N(c3ccc(-c4ccccc4)cc3)c3ccc(-c4ccc(-n5c6ccccc6c6c7ccccc7ccc65)cc4)cc3)cc2)cc1 ; c1ccc(-c2ccc(N(c3ccc(-c4ccc(-n5c6ccccc6c6c7ccccc7ccc65)cc4)cc3)c3ccccc3-c3ccccc3)cc2)cc1 ; c1ccc(-c2ccc(N(c3ccc(-c4ccc(-n5c6ccccc6c6cc7ccccc7cc65)cc4)cc3)c3cccc(-c4ccccc4)c3)cc2)cc1]

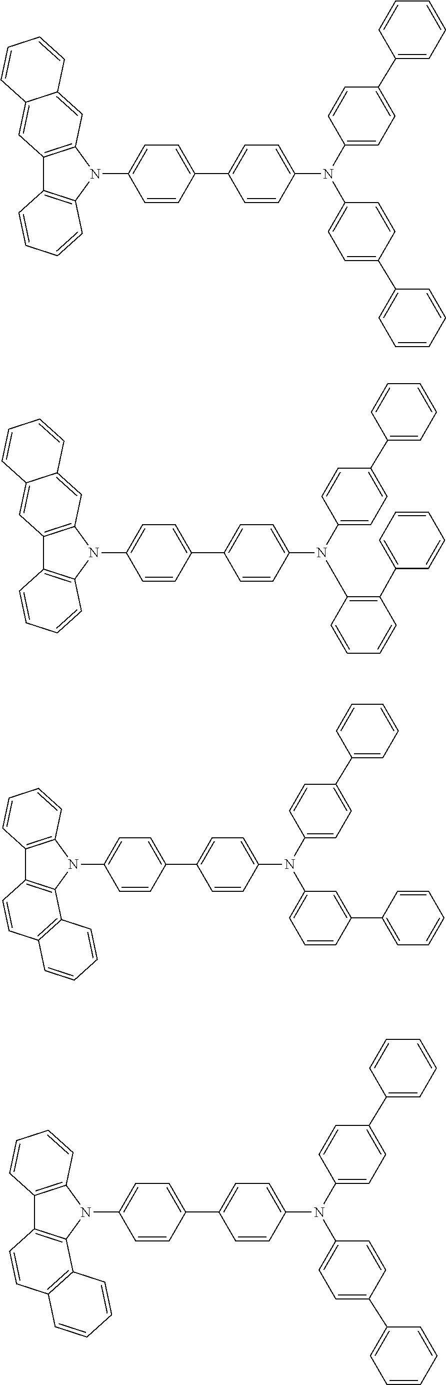

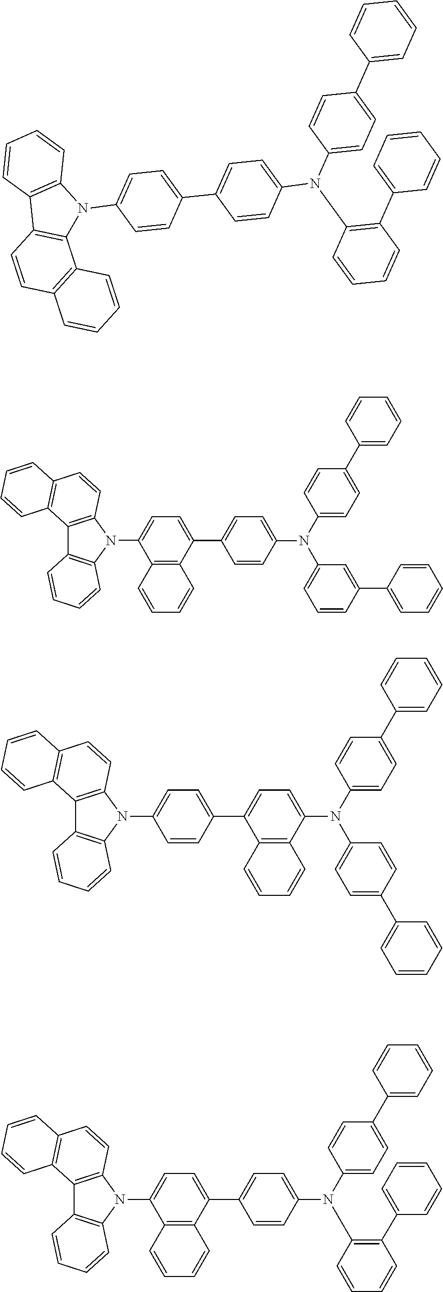 [SMILES: c1ccc(-c2ccc(N(c3ccc(-c4ccc(-n5c6ccccc6c6ccc7ccccc7c65)cc4)cc3)c3ccccc3-c3ccccc3)cc2)cc1 ; c1ccc(-c2ccc(N(c3ccc(-c4ccc(-n5c6ccccc6c6c7ccccc7ccc65)c5ccccc45)cc3)c3cccc(-c4ccccc4)c3)cc2)cc1 ; c1ccc(-c2ccc(N(c3ccc(-c4ccccc4)cc3)c3ccc(-c4ccc(-n5c6ccccc6c6c7ccccc7ccc65)cc4)c4ccccc34)cc2)cc1 ; c1ccc(-c2ccc(N(c3ccc(-c4ccc(-n5c6ccccc6c6c7ccccc7ccc65)c5ccccc45)cc3)c3ccccc3-c3ccccc3)cc2)cc1]

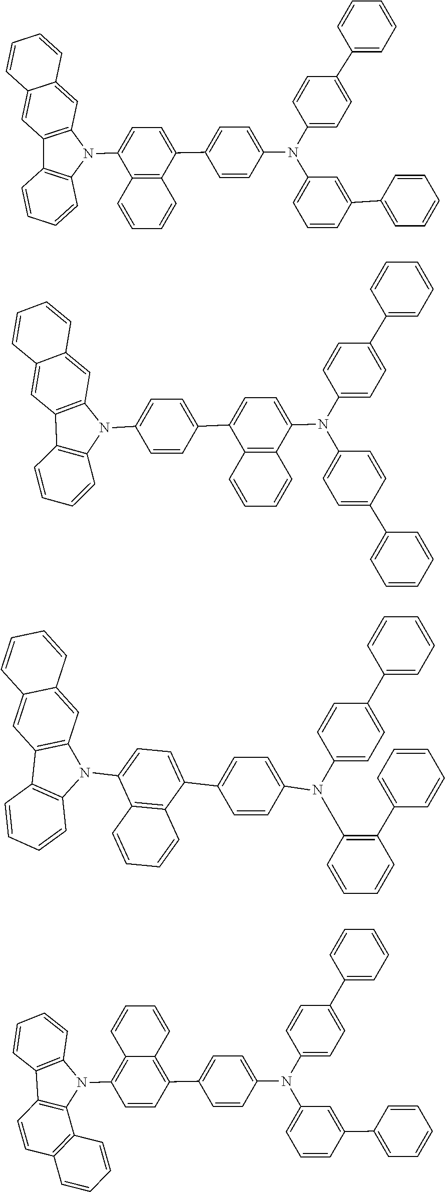

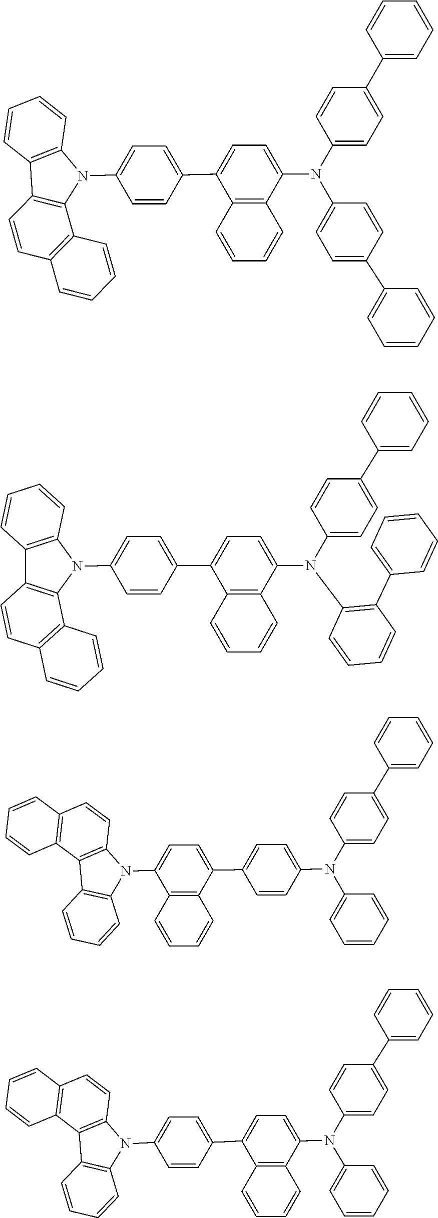

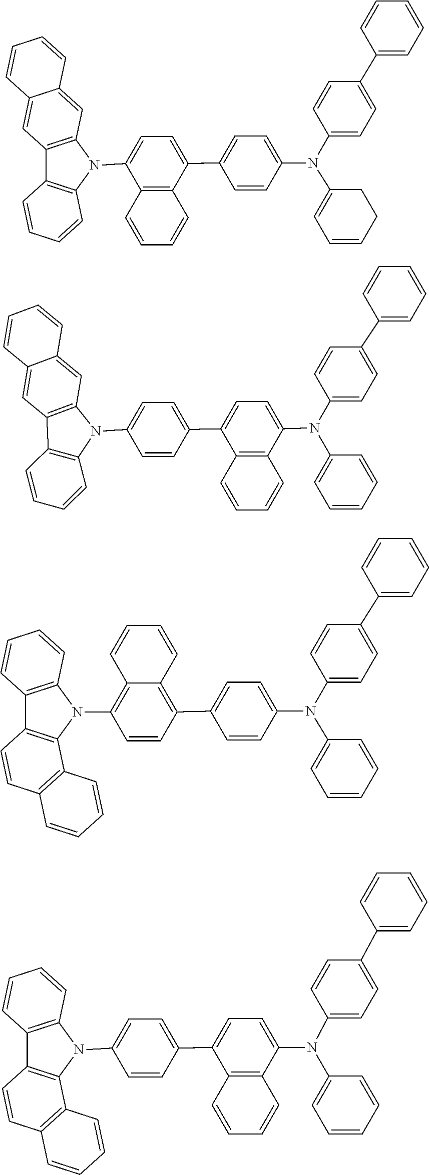

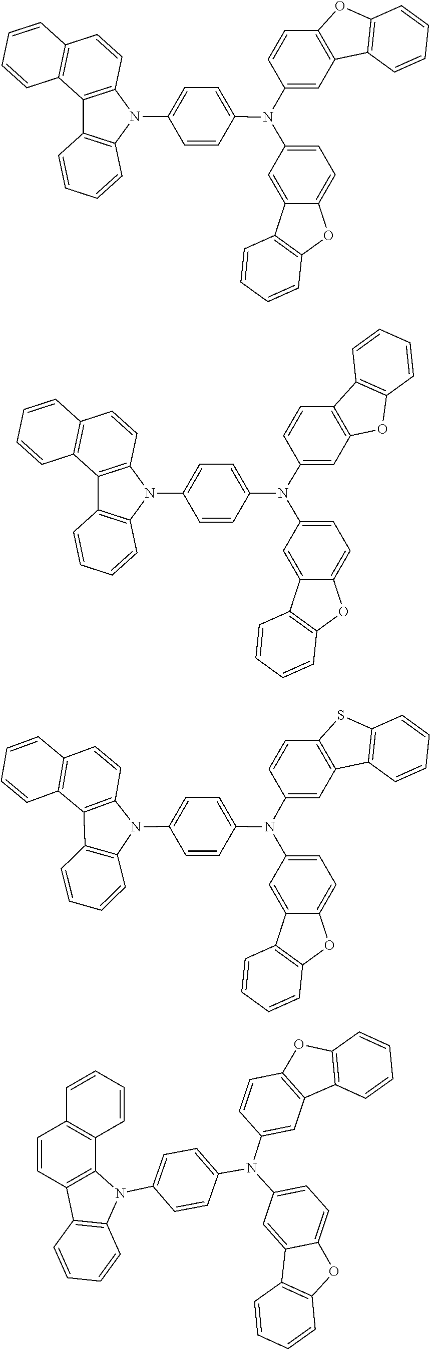

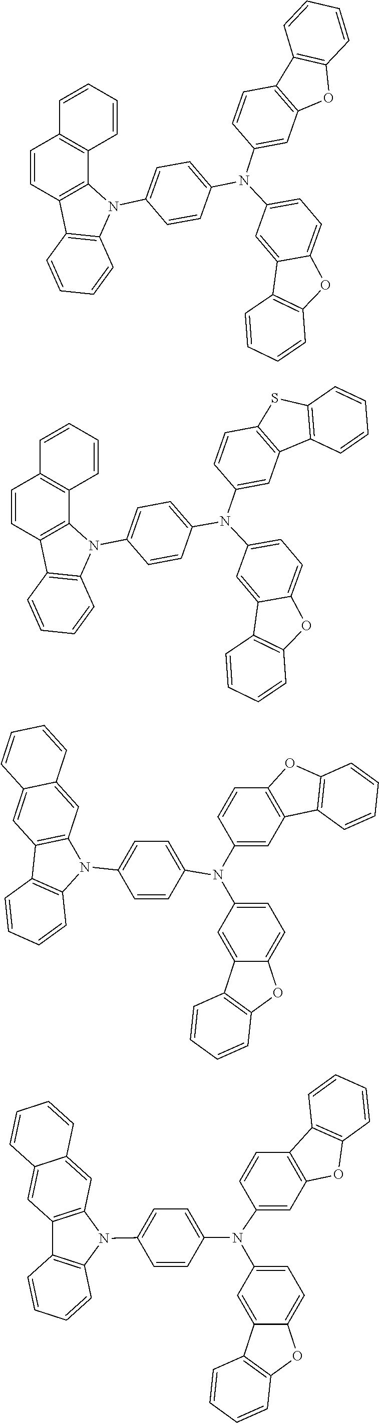

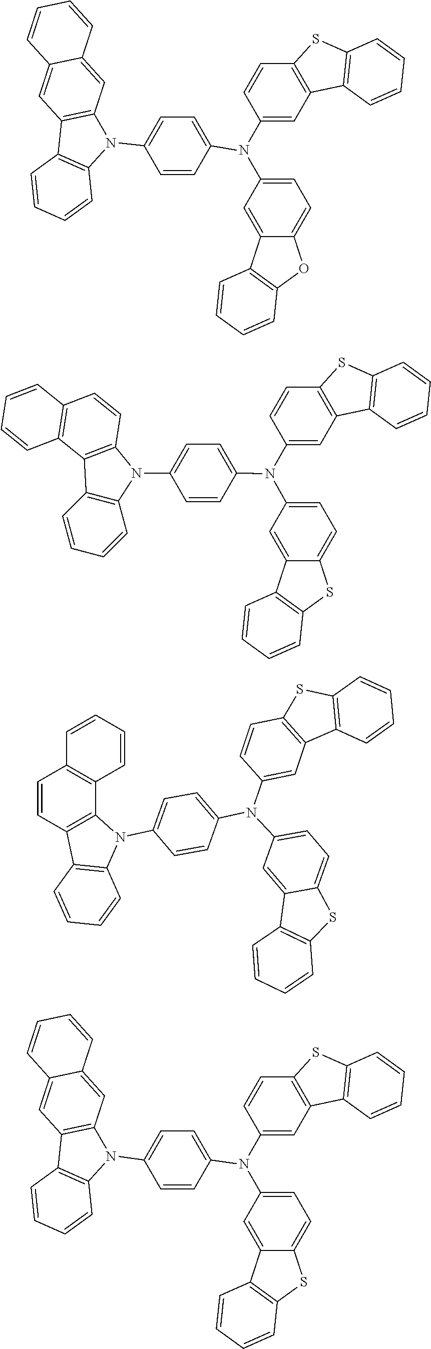

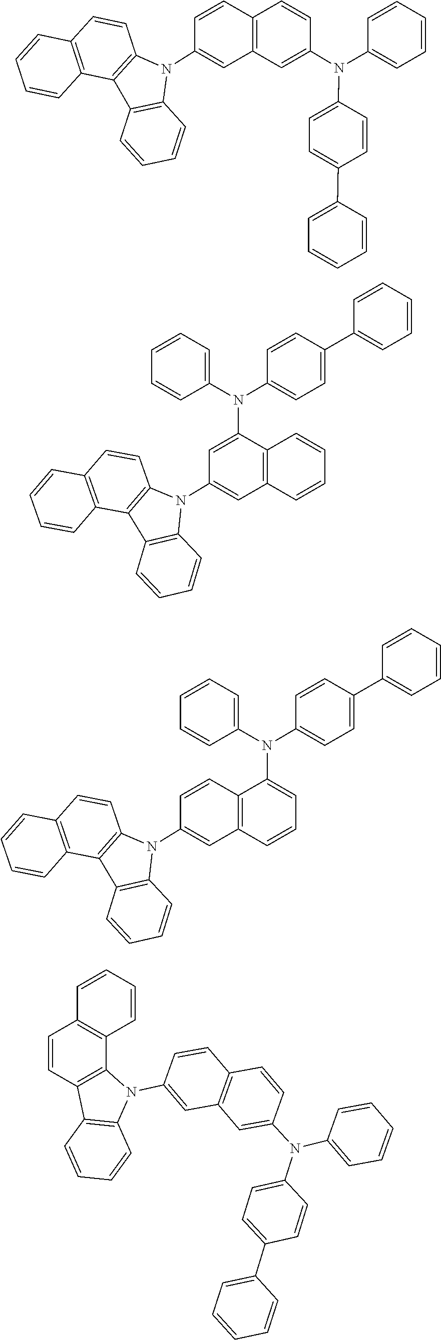

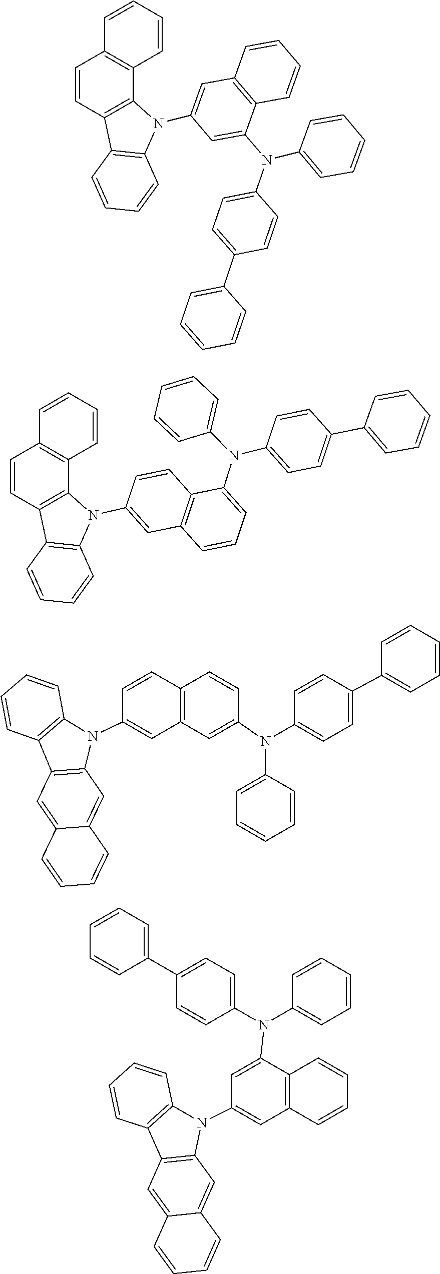

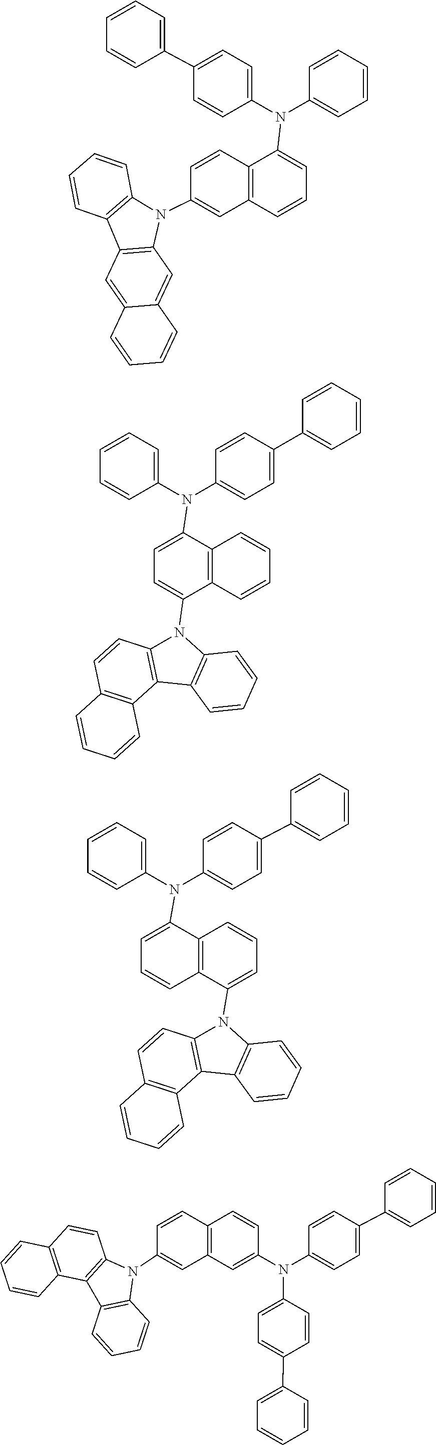 [SMILES: c1ccc(-c2ccc(N(c3ccccc3)c3cccc4cc(-n5c6ccccc6c6cc7ccccc7cc65)ccc34)cc2)cc1 ; c1ccc(-c2ccc(N(c3ccccc3)c3ccc(-n4c5ccccc5c5c6ccccc6ccc54)c4ccccc34)cc2)cc1 ; c1ccc(-c2ccc(N(c3ccccc3)c3cccc4c(-n5c6ccccc6c6c7ccccc7ccc65)cccc34)cc2)cc1 ; c1ccc(-c2ccc(N(c3ccc(-c4ccccc4)cc3)c3ccc4ccc(-n5c6ccccc6c6c7ccccc7ccc65)cc4c3)cc2)cc1]

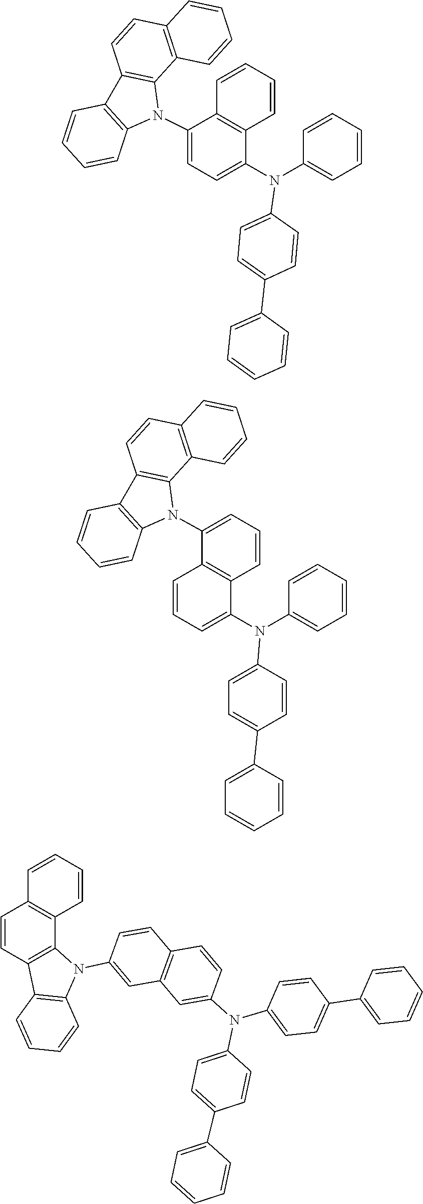

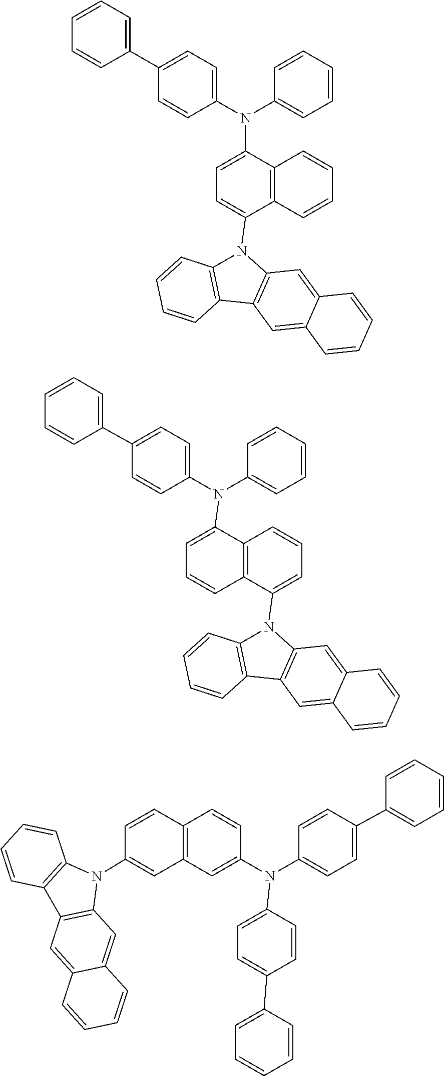

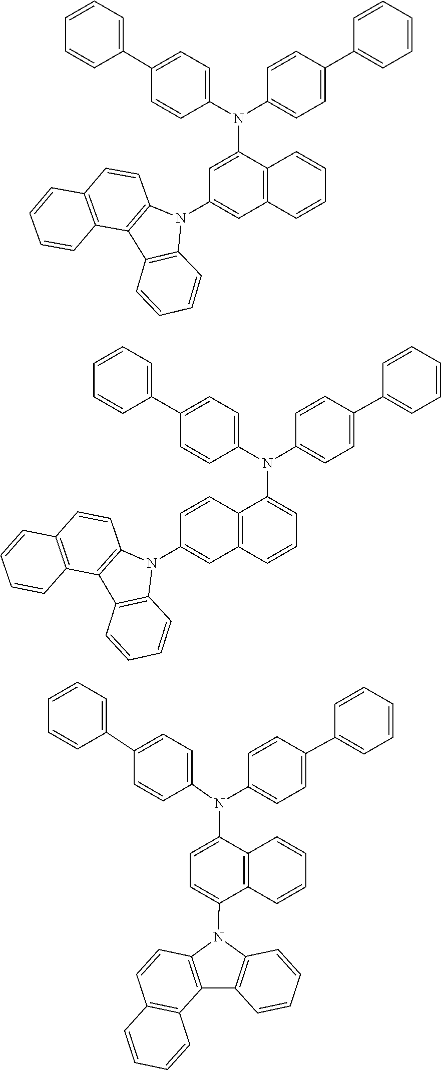

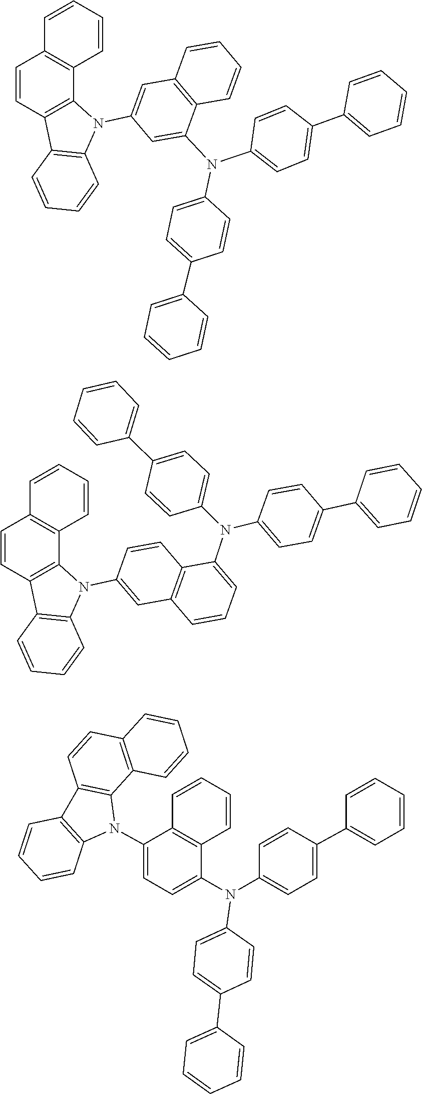 [SMILES: c1ccc(-c2ccc(N(c3ccc(-c4ccccc4)cc3)c3cc(-n4c5ccccc5c5ccc6ccccc6c54)cc4ccccc34)cc2)cc1 ; c1ccc(-c2ccc(N(c3ccc(-c4ccccc4)cc3)c3cccc4cc(-n5c6ccccc6c6ccc7ccccc7c65)ccc34)cc2)cc1 ; c1ccc(-c2ccc(N(c3ccc(-c4ccccc4)cc3)c3ccc(-n4c5ccccc5c5ccc6ccccc6c54)c4ccccc34)cc2)cc1]

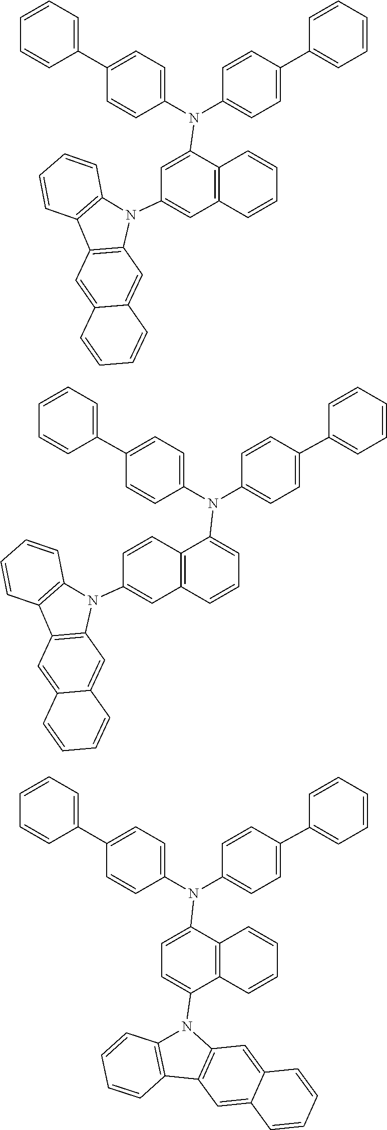

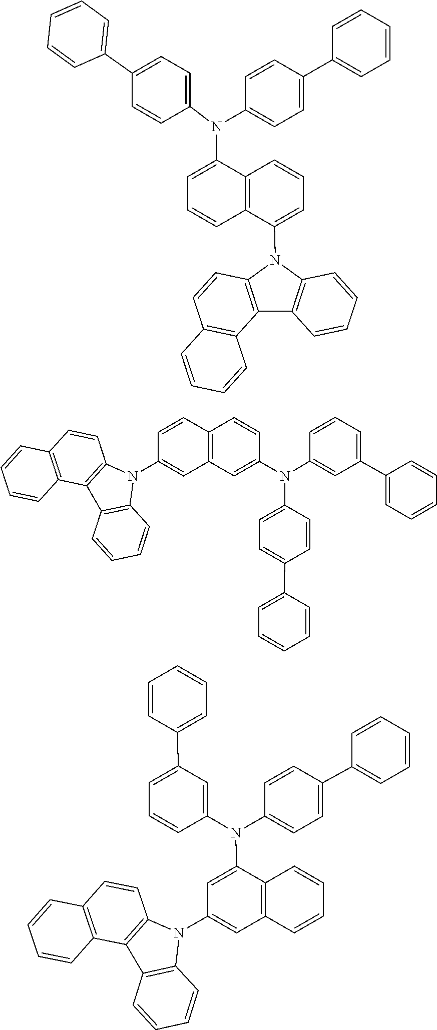

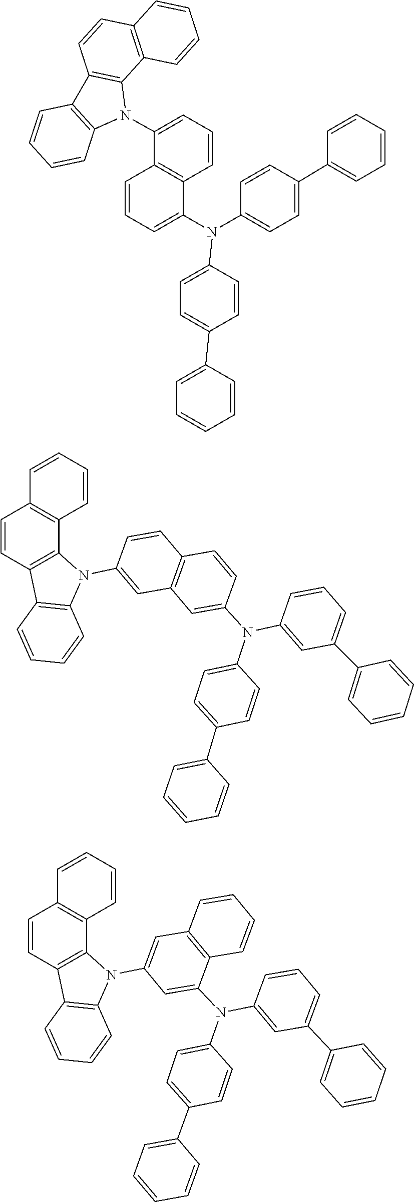 [SMILES: c1ccc(-c2ccc(N(c3ccc(-c4ccccc4)cc3)c3cccc4c(-n5c6ccccc6c6ccc7ccccc7c65)cccc34)cc2)cc1 ; c1ccc(-c2ccc(N(c3cccc(-c4ccccc4)c3)c3ccc4ccc(-n5c6ccccc6c6ccc7ccccc7c65)cc4c3)cc2)cc1 ; c1ccc(-c2ccc(N(c3cccc(-c4ccccc4)c3)c3cc(-n4c5ccccc5c5ccc6ccccc6c54)cc4ccccc34)cc2)cc1]

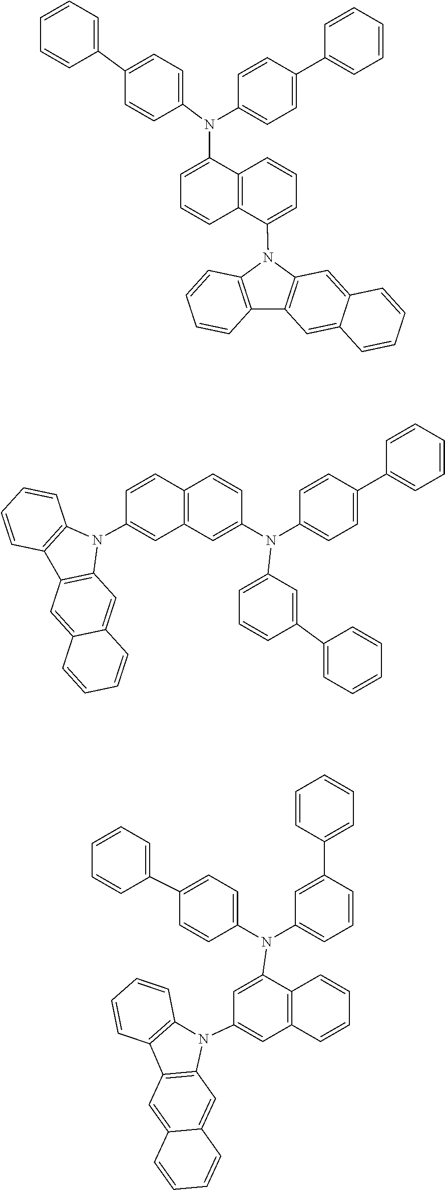 [SMILES: c1ccc(-c2ccc(N(c3ccc(-c4ccccc4)cc3)c3cccc4c(-n5c6ccccc6c6cc7ccccc7cc65)cccc34)cc2)cc1 ; c1ccc(-c2ccc(N(c3cccc(-c4ccccc4)c3)c3ccc4ccc(-n5c6ccccc6c6cc7ccccc7cc65)cc4c3)cc2)cc1 ; c1ccc(-c2ccc(N(c3cccc(-c4ccccc4)c3)c3cc(-n4c5ccccc5c5cc6ccccc6cc54)cc4ccccc34)cc2)cc1]

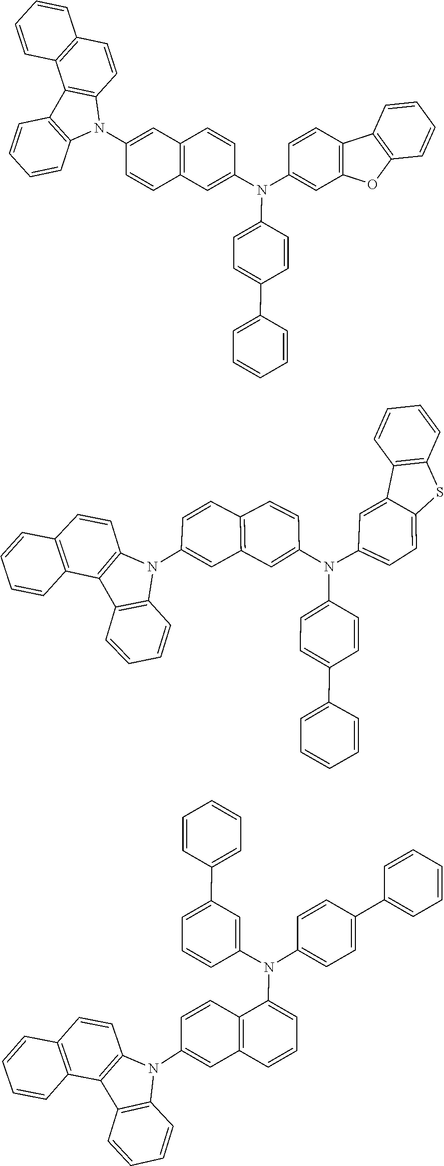 [SMILES: c1ccc(-c2ccc(N(c3ccc4cc(-n5c6ccccc6c6c7ccccc7ccc65)ccc4c3)c3ccc4c(c3)oc3ccccc34)cc2)cc1 ; c1ccc(-c2ccc(N(c3ccc4ccc(-n5c6ccccc6c6c7ccccc7ccc65)cc4c3)c3ccc4sc5ccccc5c4c3)cc2)cc1 ; c1ccc(-c2ccc(N(c3cccc(-c4ccccc4)c3)c3cccc4cc(-n5c6ccccc6c6c7ccccc7ccc65)ccc34)cc2)cc1]

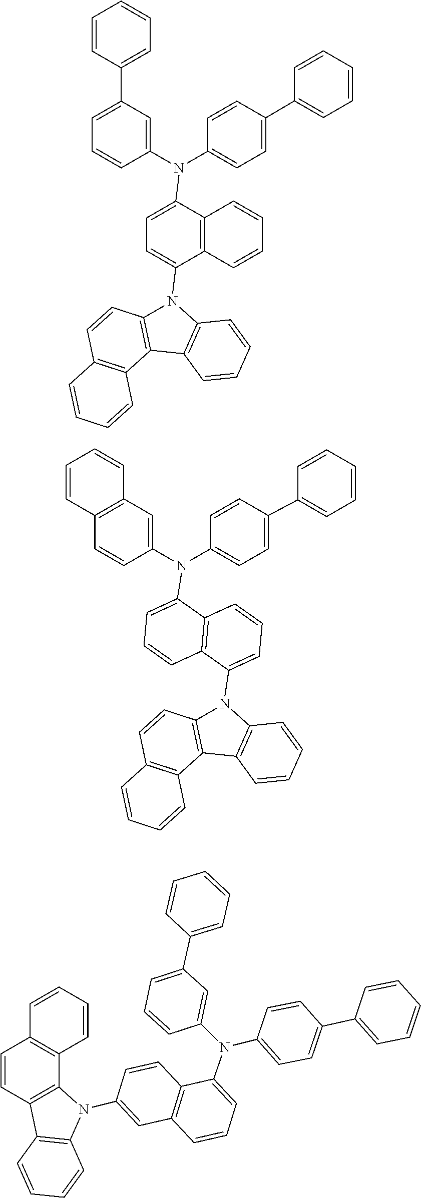

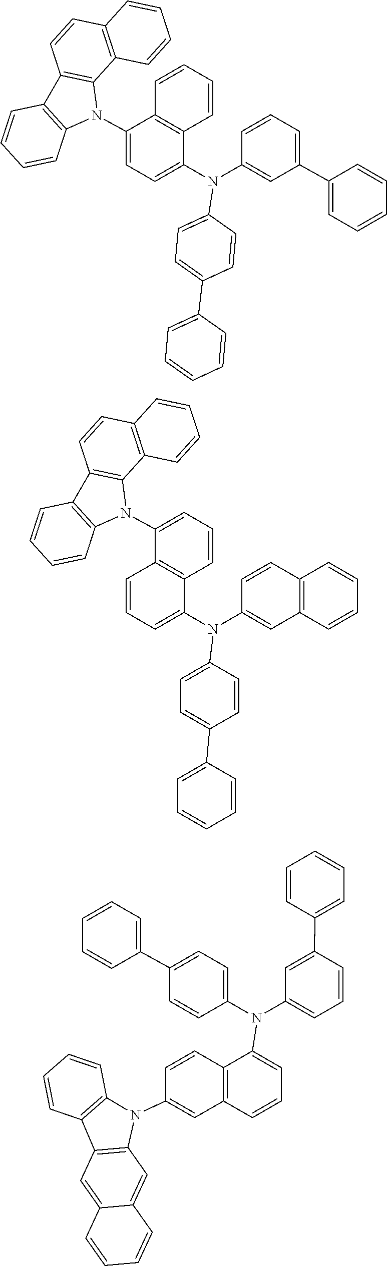

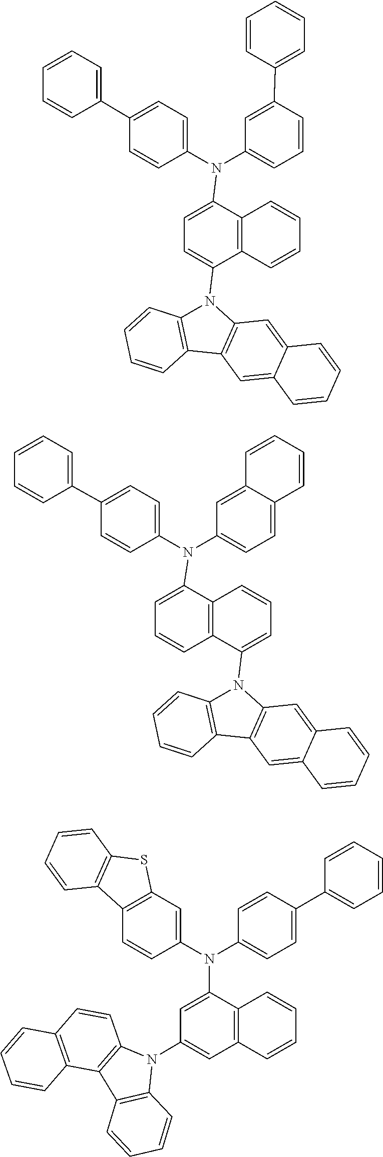

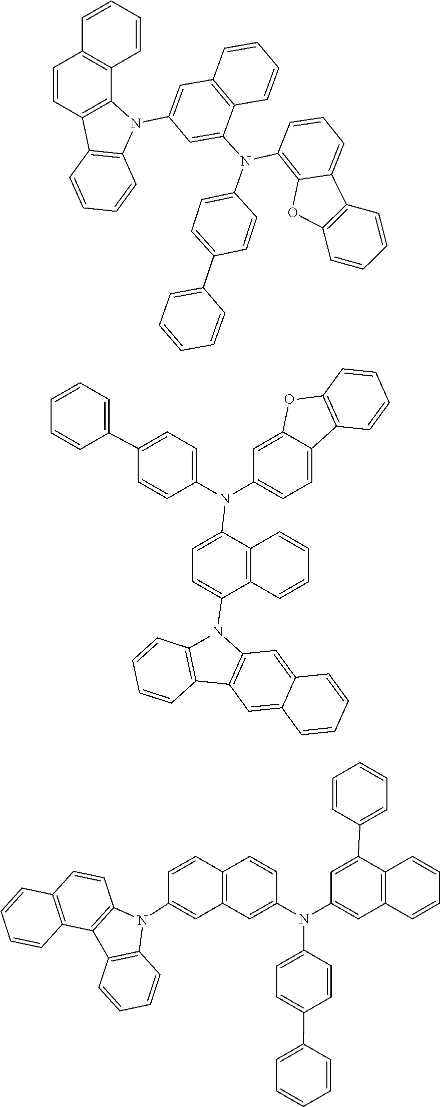

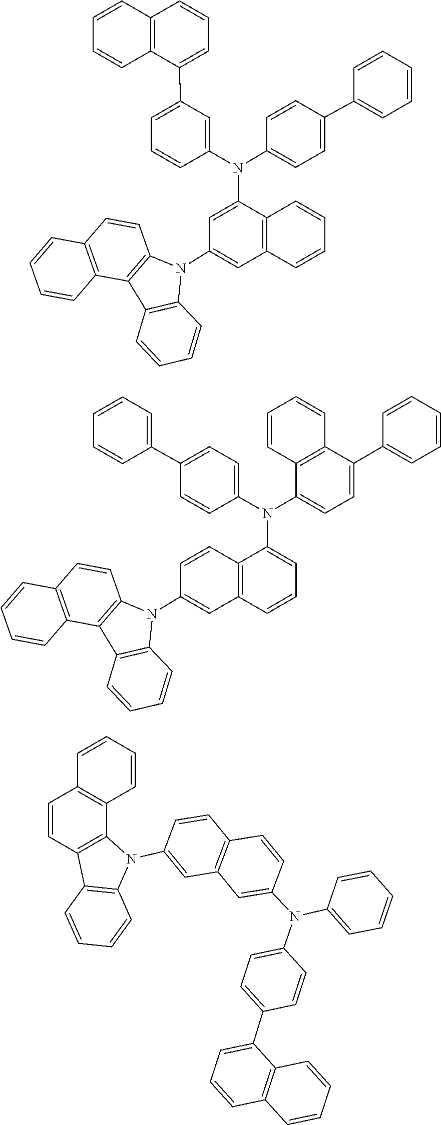

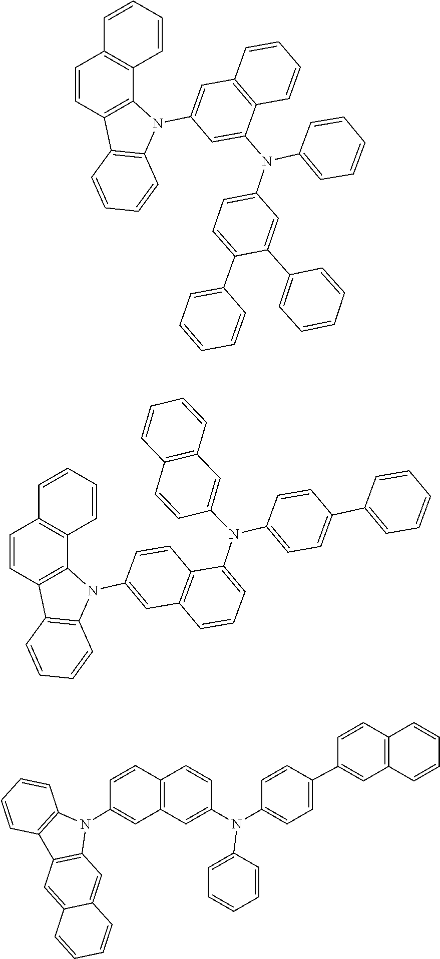 [SMILES: c1ccc(-c2ccc(N(c3ccccc3)c3cc(-n4c5ccccc5c5ccc6ccccc6c54)cc4ccccc34)cc2-c2ccccc2)cc1 ; c1ccc(-c2ccc(N(c3ccc4ccccc4c3)c3cccc4cc(-n5c6ccccc6c6ccc7ccccc7c65)ccc34)cc2)cc1 ; c1ccc(N(c2ccc(-c3ccc4ccccc4c3)cc2)c2ccc3ccc(-n4c5ccccc5c5cc6ccccc6cc54)cc3c2)cc1]

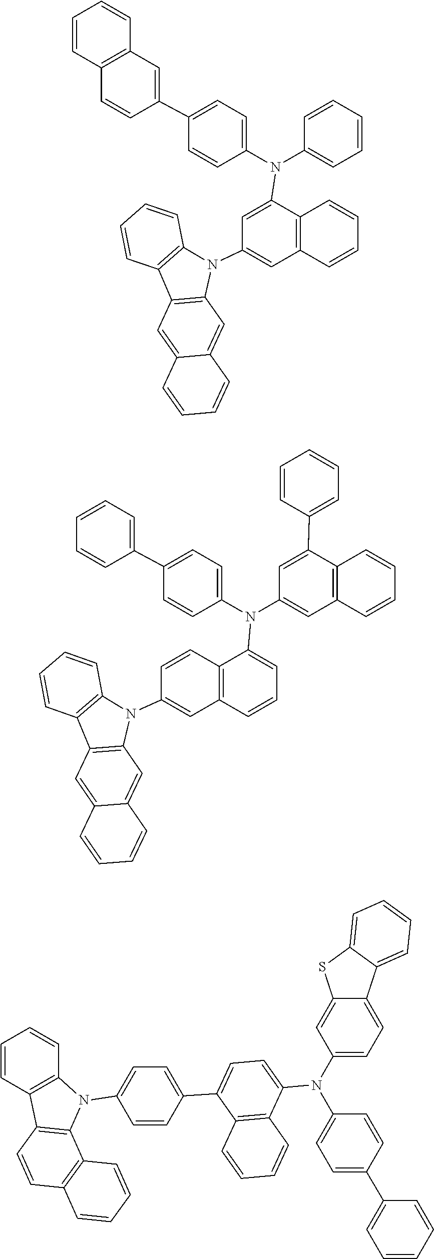 [SMILES: c1ccc(N(c2ccc(-c3ccc4ccccc4c3)cc2)c2cc(-n3c4ccccc4c4cc5ccccc5cc43)cc3ccccc23)cc1 ; c1ccc(-c2ccc(N(c3cc(-c4ccccc4)c4ccccc4c3)c3cccc4cc(-n5c6ccccc6c6cc7ccccc7cc65)ccc34)cc2)cc1 ; c1ccc(-c2ccc(N(c3ccc4c(c3)sc3ccccc34)c3ccc(-c4ccc(-n5c6ccccc6c6ccc7ccccc7c65)cc4)c4ccccc34)cc2)cc1]

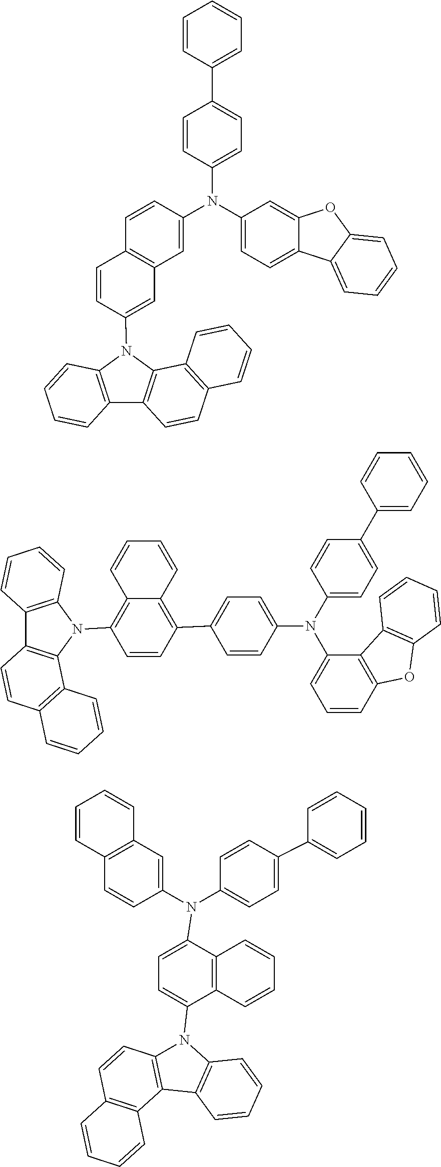

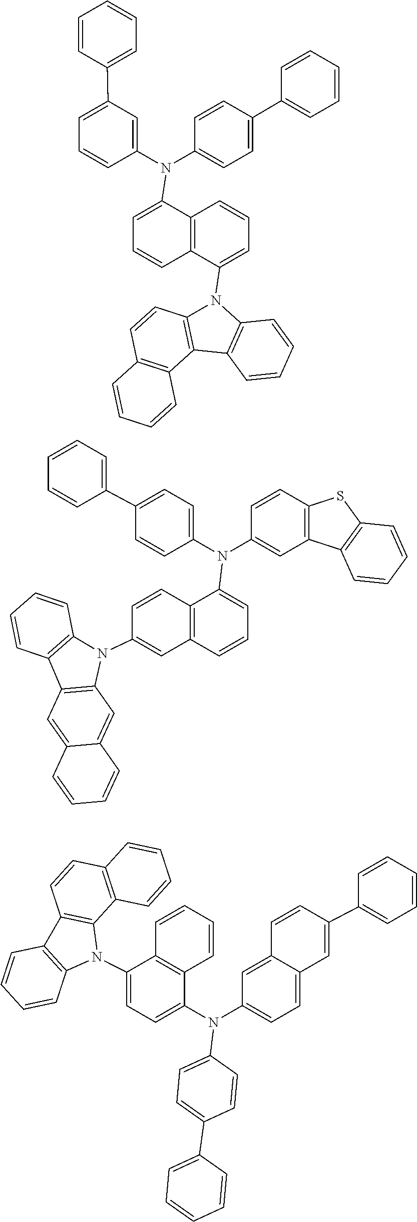

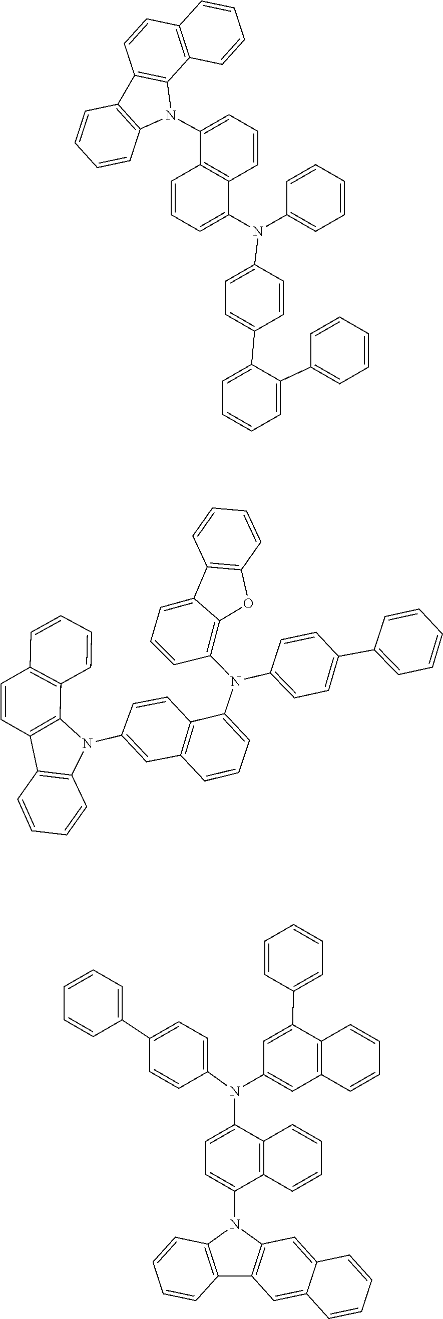 [SMILES: c1ccc(-c2ccccc2-c2ccc(N(c3ccccc3)c3cccc4c(-n5c6ccccc6c6ccc7ccccc7c65)cccc34)cc2)cc1 ; c1ccc(-c2ccc(N(c3cccc4cc(-n5c6ccccc6c6ccc7ccccc7c65)ccc34)c3cccc4c3oc3ccccc34)cc2)cc1 ; c1ccc(-c2ccc(N(c3cc(-c4ccccc4)c4ccccc4c3)c3ccc(-n4c5ccccc5c5cc6ccccc6cc54)c4ccccc34)cc2)cc1]

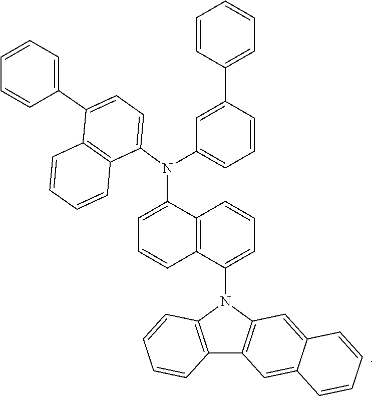 [SMILES: c1ccc(-c2cccc(N(c3ccc(-c4ccccc4)c4ccccc34)c3cccc4c(-n5c6ccccc6c6cc7ccccc7cc65)cccc34)c2)cc1]

The compound of Chemical Formula 2 can be prepared by, for example, a preparation method as shown in Reaction Scheme 2 below.

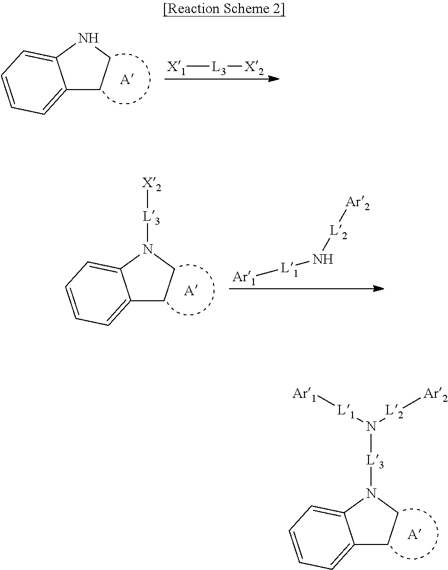

In Reaction Scheme 2, the definitions of other substituents except for X′₁ and X′₂ are the same as described above. X′₁ and X′₂ are each independently halogen, and more preferably bromo or chloro.

The Reaction Scheme 2 is an amine substitution reaction, and preferably performed in the presence of a palladium catalyst and a base, and the reactor for the amine substitution reaction can be changed as known in the art. The preparation method can be more specifically described in the Preparation Examples described below.

In the light emitting layer, a weight ratio of the compound of Chemical Formula 1 and the compound of Chemical Formula 2 is 1:99 to 99:1, 5:95 to 95:5, or 10:90 to 90:10.

The dopant material is not particularly limited as long as it is a material used in an organic light emitting device. For example, the dopant material includes an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

Specific examples of the dopant material include, but are not limited to, the following compounds:

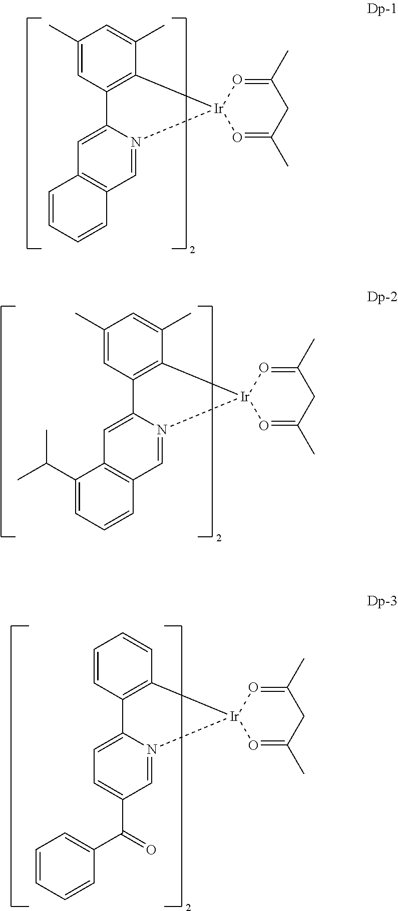

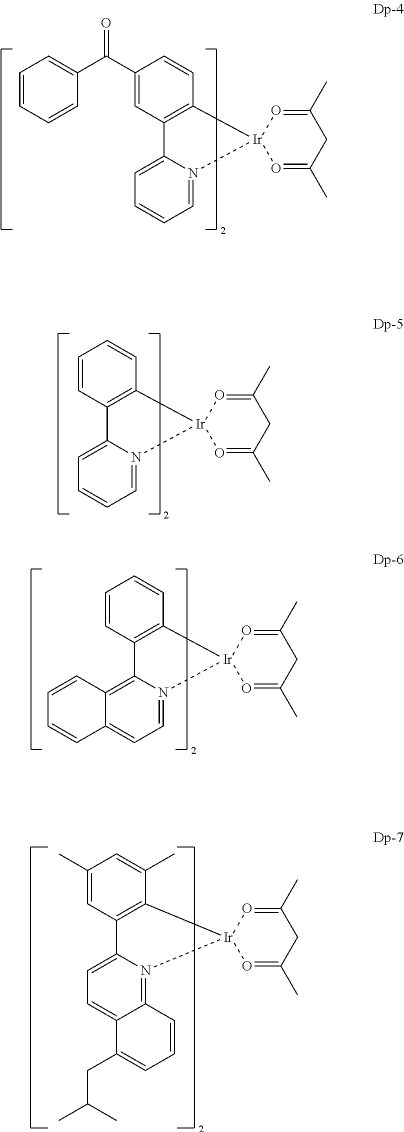

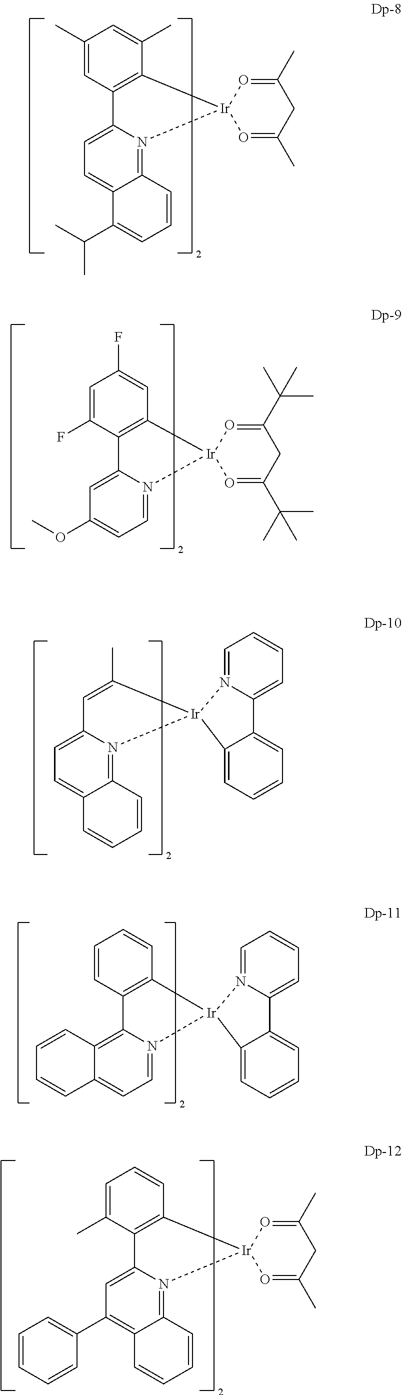

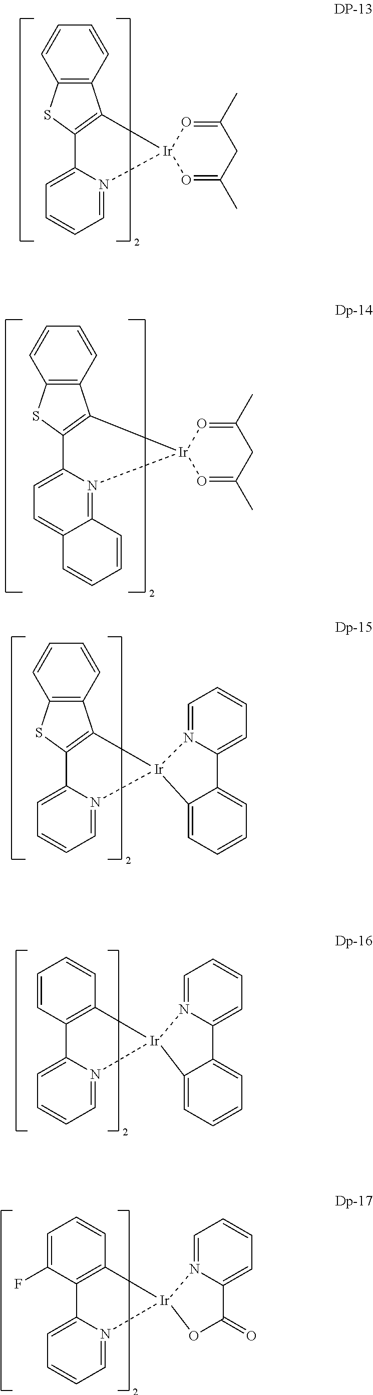

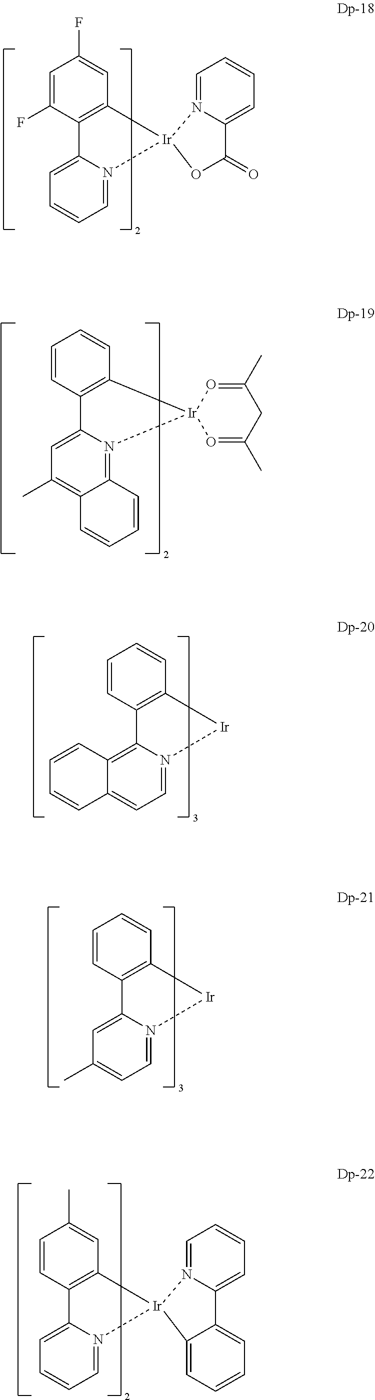

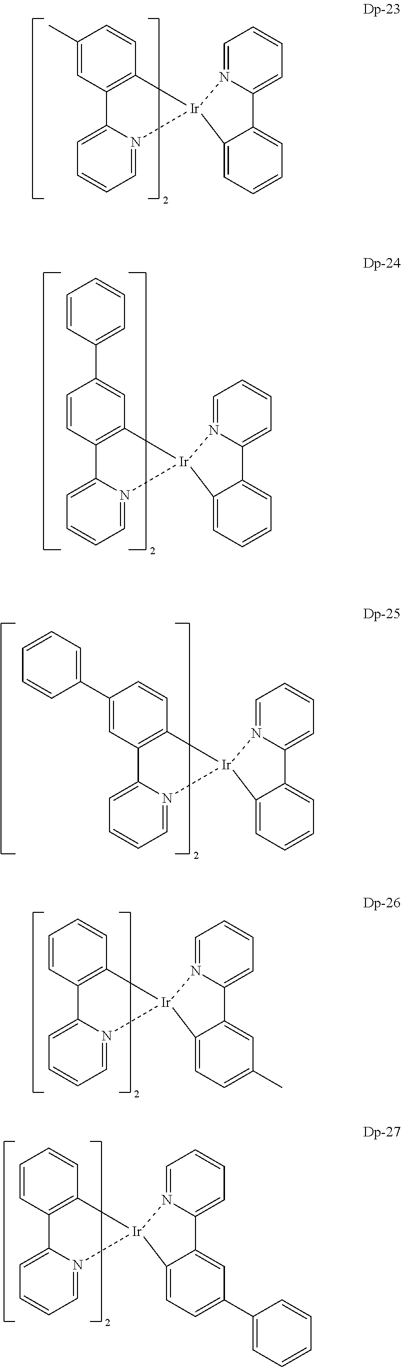

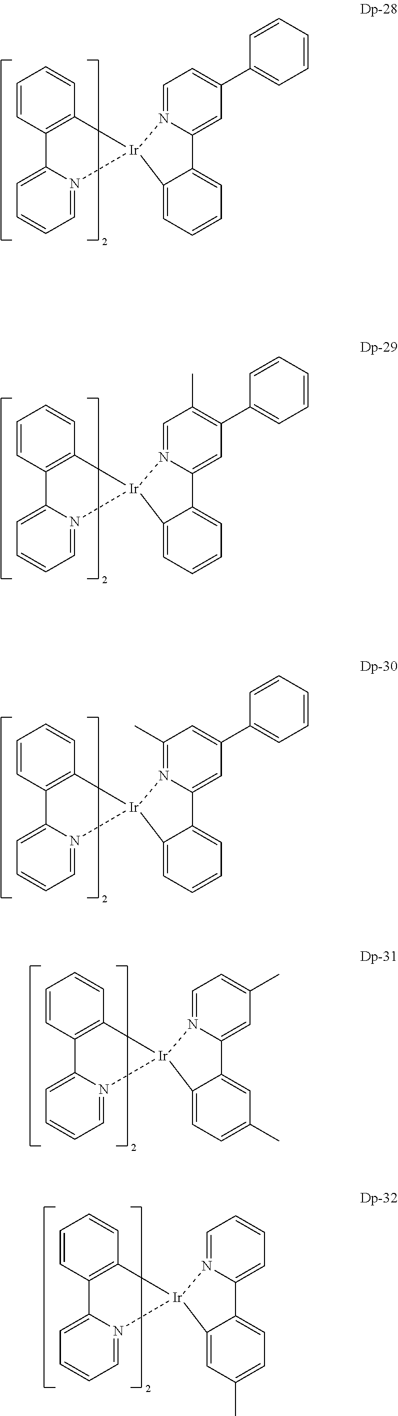

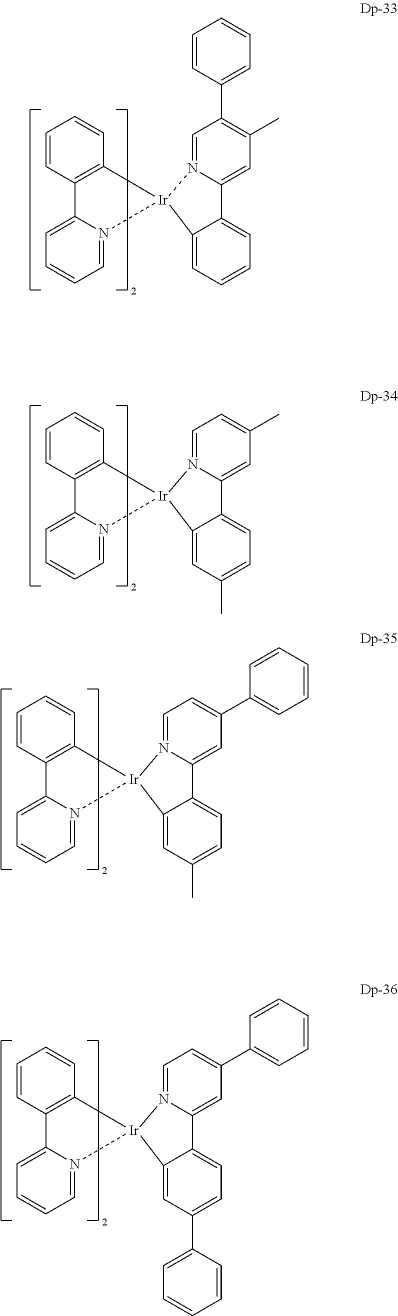

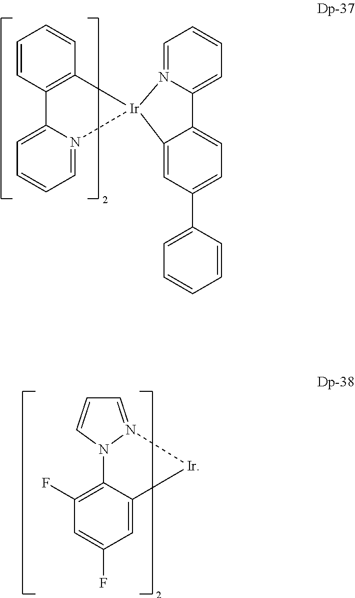

### Hole Transport Layer

The organic light emitting device according to the present disclosure can include a hole transport layer between the light emitting layer and the anode.

The hole transport layer is a layer that receives holes from a hole injection layer and transports the holes to the light emitting layer. The hole transport material is suitably a material having large mobility to the holes, which can receive holes from the anode or the hole injection layer and transfer the holes to the light emitting layer.

Specific examples of the hole transport material include an arylamine-based organic material, a conductive polymer, a block copolymer in which a conjugate portion and a non-conjugate portion are present together, and the like, but are not limited thereto.

### Hole Injection Layer

The organic light emitting device according to the present disclosure can further include a hole injection layer between the anode and the hole transport layer, if necessary.

The hole injection layer is a layer for injecting holes from the electrode, and the hole injection material is preferably a compound which can transport the holes, thus has a hole-injecting effect in the anode and an excellent hole-injecting effect to the light emitting layer or the light emitting material, prevents excitons produced in the light emitting layer from moving to an electron injection layer or the electron injection material, and is excellent in the ability to form a thin film. It is preferable that a HOMO (highest occupied molecular orbital) of the hole injection material is between the work function of the anode material and a HOMO of a peripheral organic material layer.

Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

### Electron Blocking Layer

The organic light emitting device according to the present disclosure can include an electron blocking layer between a hole transport layer and a light emitting layer, if necessary.

The electron blocking layer prevents electrons injected from the cathode from being transferred to the hole transport layer without recombination in the light emitting layer, and is also called an electron suppressing layer. A material having the electron affinity lower than that of the electron transport layer is preferable for the electron blocking layer.

### Electron Transport Layer

The organic light emitting device according to the present disclosure can include an electron transport layer between the light emitting layer and the cathode.

The electron transport layer is a layer which receives electrons from a cathode or an electron injection layer formed on the cathode and transports the electrons to a light emitting layer, and can suppress the transfer of holes in the light emitting layer. An electron transport material is suitably a material which can receive electrons well from a cathode and transport the electrons to a light emitting layer, and has large mobility for electrons.

Specific examples of the electron transport material include an Al complex of 8-hydroxyquinoline, a complex including Alq₃, an organic radical compound, a hydroxyflavone-metal complex, and the like, but are not limited thereto. The electron transport layer can be used with any desired cathode material, as used in the related art. In particular, appropriate examples of the cathode material are typical materials having a low work function, followed by an aluminum layer or a silver layer. Specific examples thereof include cesium, barium, calcium, ytterbium, and samarium, in each case followed by an aluminum layer or a silver layer.

### Electron Injection Layer

The organic light emitting device according to the present disclosure can further include an electron injection layer between the electron transport layer and the cathode, if necessary.

The electron injection layer is a layer which injects electrons from an electrode, and the electron injection material is preferably a compound which can transport electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film.

Specific examples of the material that can be used as the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxy-quinolinato lithium, bis(8-hydroxyquinolinato)zinc, bis(8-hydroxyquinolinato)-copper, bis(8-hydroxyquinolinato)manganese, tris(8-hydroxyquinolinato)-aluminum, tris(2-methyl-8-hydroxyquinolinato)aluminum, tris(8-hydroxy-quinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)-(1-naphtholato)aluminum, bis(2-methyl-8-quinolinato)(2-naphtholato)gallium, and the like, but are not limited thereto.

According to one embodiment of the present disclosure, the electron transport material and the electron injection material can be simultaneously deposited to form an electron injection and transport layer as a single layer.

### Hole Blocking Layer

The organic light emitting device according to the present disclosure includes a hole blocking layer between the electron transport layer and the light emitting layer, if necessary.

The hole blocking layer prevents holes injected from the anode from being transferred to the electron transport layer without recombination in the light emitting layer, and a material having high ionization energy is preferable for the hole blocking layer.

### Organic Light Emitting Device

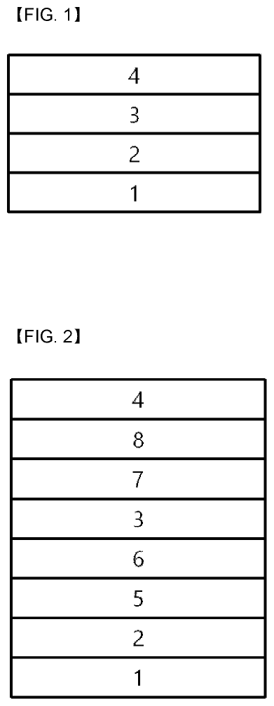

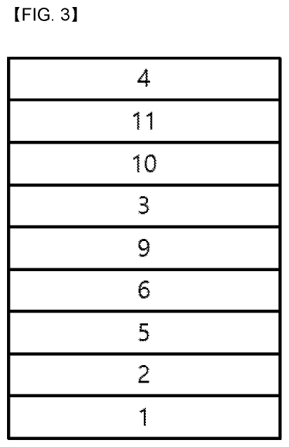

A structure of the organic light emitting device according to the present disclosure is illustrated in FIG. 1. FIG. 1 shows an example of an organic light emitting device including a substrate 1, an anode 2, a light emitting layer 3, and a cathode 4. FIG. 2 shows an example of an organic light emitting device including a substrate 1, an anode 2, a hole injection layer 5, a hole transport layer 6, a light emitting layer 3, an electron transport layer 7, an electron injection layer 8, and a cathode 4. FIG. 3 shows an example of an organic light emitting device including a substrate 1, an anode 2, a hole injection layer 5, a hole transport layer 6, an electron blocking layer 9, a light emitting layer 3, a hole blocking layer 10, an electron injection and transport layer 11, and a cathode 4.

The organic light emitting device according to the present disclosure can be manufactured by sequentially laminating the above-described components. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming the above-mentioned respective layers thereon, and then depositing a material that can be used as the cathode thereon.

In addition to such a method, the organic light emitting device can be manufactured by sequentially depositing the above-described components from a cathode material to an anode material in the reverse order on a substrate (WO 2003/012890). Further, the light emitting layer can be formed using the host and the dopant by a solution coating method as well as a vacuum deposition method. Herein, the solution coating method means a spin coating, a dip coating, a doctor blading, an inkjet printing, a screen printing, a spray method, a roll coating, or the like, but is not limited thereto.

Meanwhile, the organic light emitting device according to the present disclosure can be a front side emission type, a backside emission type, or a double-sided emission type according to the used material.

The preparation of the organic light emitting device will be described in detail in the following examples. However, these examples are presented for illustrative purposes only, and are not intended to limit the scope of the present disclosure.

### PREPARATION EXAMPLES

### Preparation Example 1-1

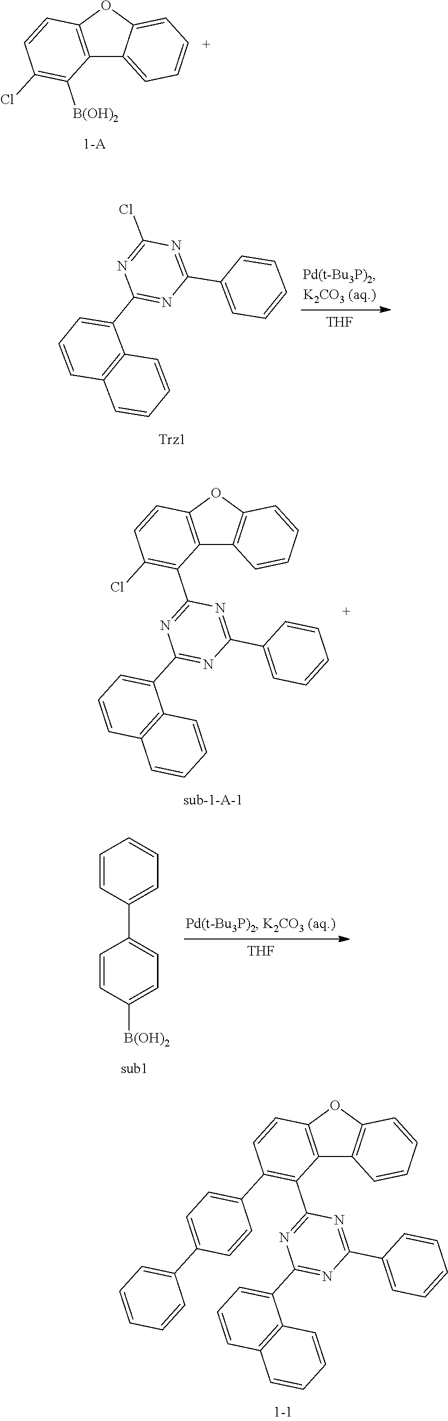

Compound 1-A (15 g, 60.9 mmol) and Trz1 (19.3 g, 60.9 mmol) were added to 300 ml of THF under a nitrogen atmosphere, and the mixture was stirred and refluxed. Then, potassium carbonate (16.8 g, 121.7 mmol) was dissolved in 50 ml of water, and then added thereto. Thereafter, it was stirred sufficiently, followed by adding bis(tri-tert-butylphosphine)palladium(0) (0.3 g, 0.6 mmol). After 12 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.9 g of sub1-A-1 (yield 71%, MS: [M+H]+=484).

sub1-A-1 (15 g, 31 mmol) and sub1 (6.1 g, 31 mmol) were added to 300 ml of THF under a nitrogen atmosphere, and the mixture was stirred and refluxed. Then, potassium carbonate (8.6 g, 62 mmol) was dissolved in 26 ml of water, and then added thereto. Thereafter, it was stirred sufficiently, followed by adding bis(tri-tert-butylphosphine)palladium(0) (0.2 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.3 g of Compound 1-1 (yield 66%, MS: [M+H]+=602).

### Preparation Example 1-2

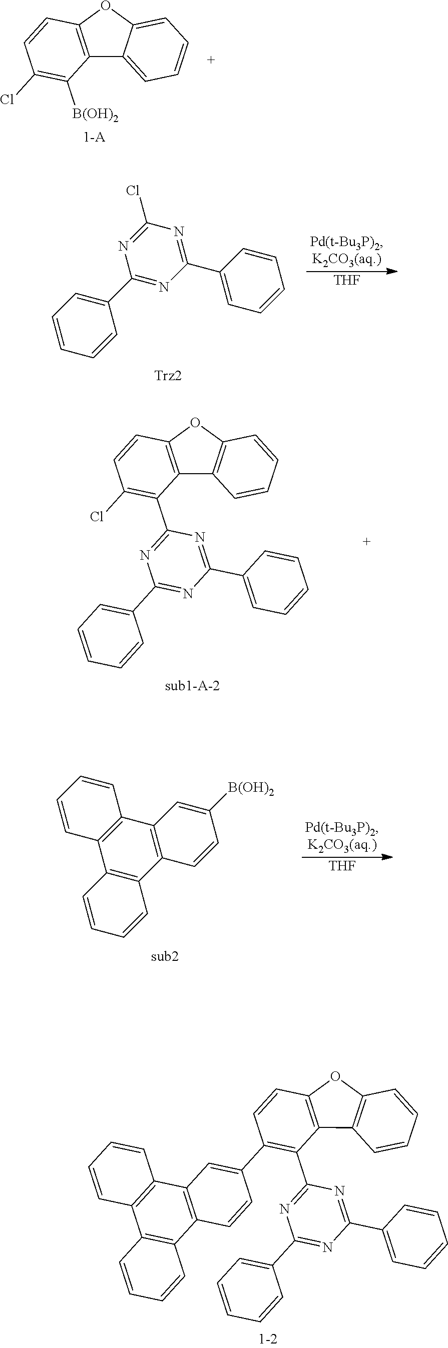

Compound 1-A (15 g, 60.9 mmol) and Trz2 (16.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 19.5 g of sub1-A-2 (yield 74%, MS: [M+H]+=434).

sub1-A-2 (15 g, 34.6 mmol) and sub2 (9.4 g, 34.6 mmol) were added to 300 ml of THF under a nitrogen atmosphere, and the mixture was stirred and refluxed. Then, potassium carbonate (9.6 g, 69.1 mmol) was dissolved in 29 ml of water, and then added thereto. Thereafter, it was stirred sufficiently, followed by adding bis(tri-tert-butylphosphine)palladium(0) (0.2 g, 0.3 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.3 g of Compound 1-2 (yield 66%, MS: [M+H]+=626).

### Preparation Example 1-3

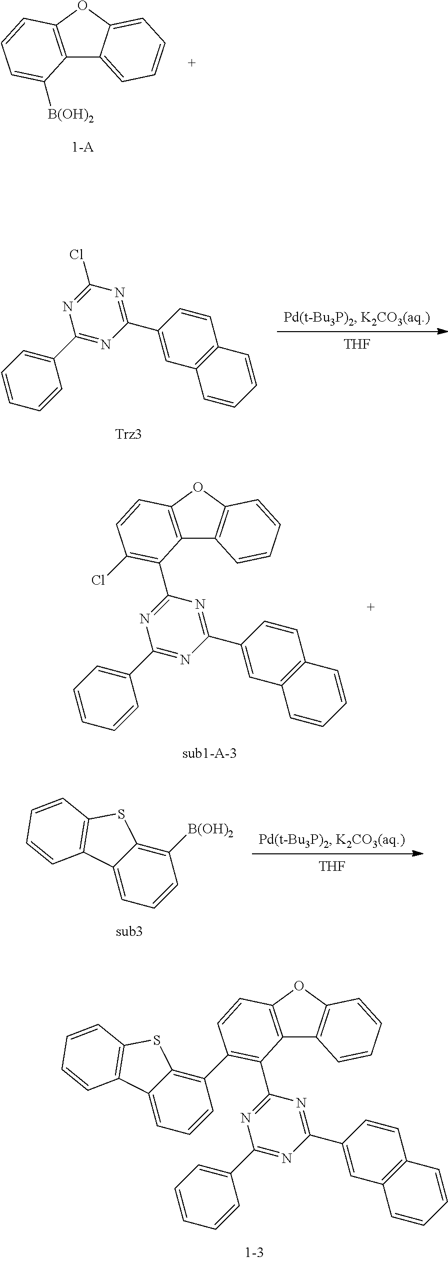

Compound 1-A (15 g, 60.9 mmol) and Trz3 (19.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23.2 g of sub1-A-3 (yield 79%, MS: [M+H]+=484).

sub1-A-3 (15 g, 31 mmol) and sub3 (7.1 g, 31 mmol) were added to 300 ml of THF under a nitrogen atmosphere, and the mixture was stirred and refluxed. Then, potassium carbonate (8.6 g, 62 mmol) was dissolved in 26 ml of water, and then added thereto. Thereafter, it was stirred sufficiently, followed by adding bis(tri-tert-butylphosphine)palladium(0) (0.2 g, 0.3 mmol). After 12 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.9 g of Compound 1-3 (yield 66%, MS: [M+H]+=632).

### Preparation Example 1-4

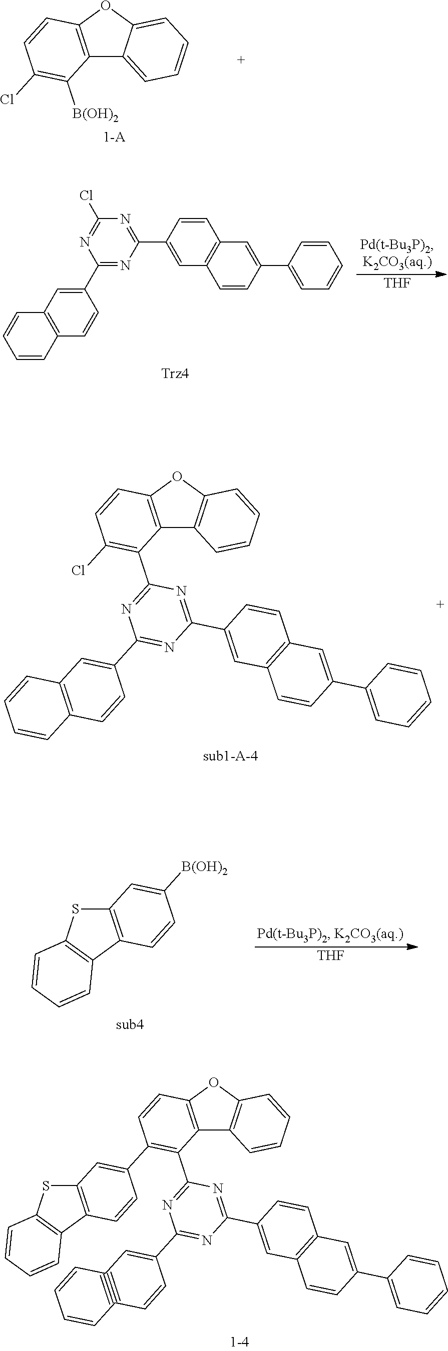

Compound 1-A (15 g, 60.9 mmol) and Trz4 (27 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 26 g of sub1-A-4 (yield 70%, MS: [M+H]+=610).

sub1-A-4 (15 g, 24.6 mmol) and sub4 (5.6 g, 24.6 mmol) were added to 300 ml of THF under a nitrogen atmosphere, and the mixture was stirred and refluxed. Then, potassium carbonate (6.8 g, 49.2 mmol) was dissolved in 20 ml of water, and then added thereto. Thereafter, it was stirred sufficiently, followed by adding bis(tri-tert-butylphosphine)palladium(0) (0.1 g, 0.2 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.2 g of Compound 1-4 (yield 60%, MS: [M+H]+=758).

### Preparation Example 1-5

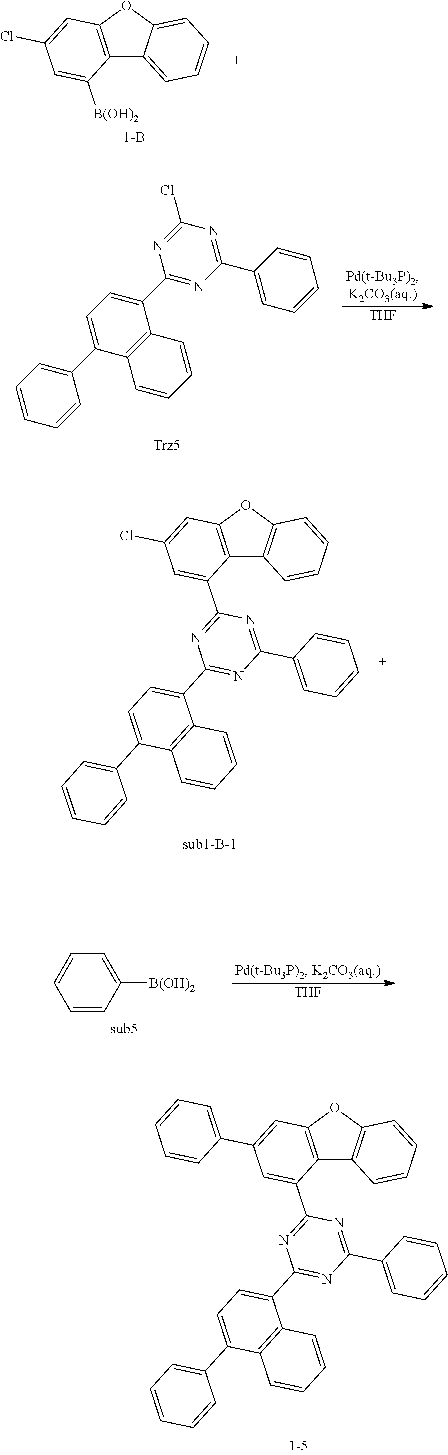

Compound 1-B (15 g, 60.9 mmol) and Trz5 (24 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 26.2 g of sub1-B-1 (yield 77%, MS: [M+H]+=560).

sub1-B-1 (15 g, 26.8 mmol) and sub5 (3.3 g, 26.8 mmol) were added to 300 ml of THF under a nitrogen atmosphere, and the mixture was stirred and refluxed. Then, potassium carbonate (7.4 g, 53.6 mmol) was dissolved in 22 ml of water, and then added thereto. Thereafter, it was stirred sufficiently, followed by adding bis(tri-tert-butylphosphine)palladium(0) (0.1 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.9 g of Compound 1-5 (yield 80%, MS: [M+H]+=602).

### Preparation Example 1-6

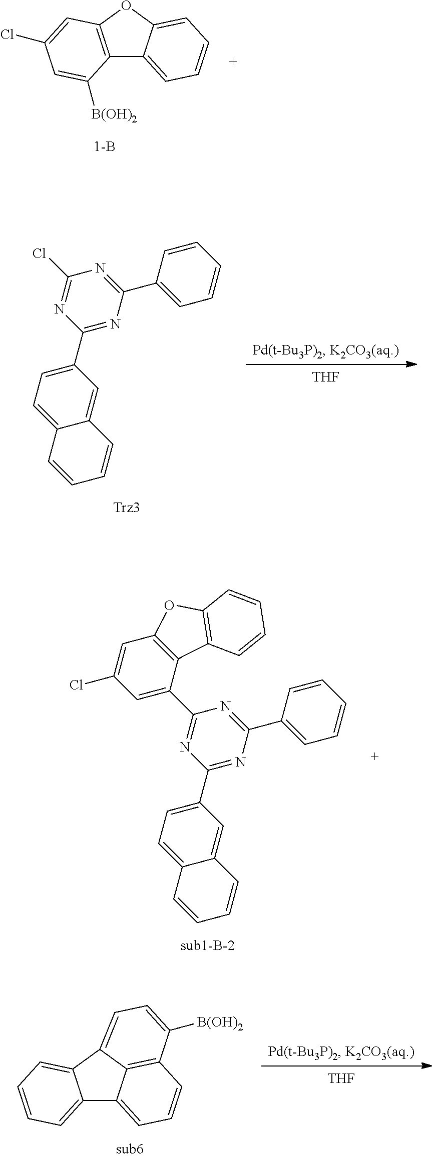 [SMILES: OB(O)c1cc(Cl)cc2oc3ccccc3c12 ; "THF" ; Clc1nc(-c2ccccc2)nc(-c2ccc3ccccc3c2)n1 ; Clc1cc(-c2nc(-c3ccccc3)nc(-c3ccc4ccccc4c3)n2)c2c(c1)oc1ccccc12 ; "THF" ; OB(O)c1ccc2c3c(cccc13)-c1ccccc1-2]

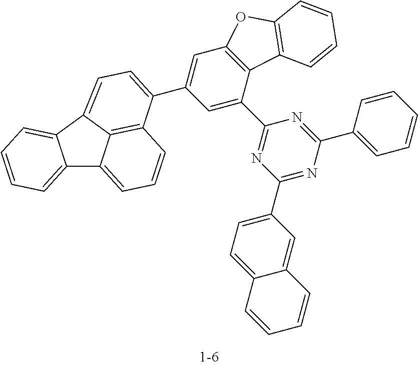 [SMILES: c1ccc(-c2nc(-c3ccc4ccccc4c3)nc(-c3cc(-c4ccc5c6c(cccc46)-c4ccccc4-5)cc4oc5ccccc5c34)n2)cc1]

Compound 1-B (15 g, 60.9 mmol) and Trz3 (19.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 18.2 g of sub1-B-2 (yield 62%, MS: [M+H]+=484).

sub1-B-2 (15 g, 31 mmol) and sub6 (7.6 g, 31 mmol) were added to 300 ml of THF under a nitrogen atmosphere, and the mixture was stirred and refluxed. Then, potassium carbonate (8.6 g, 62 mmol) was dissolved in 26 ml of water, and then added thereto. Thereafter, it was stirred sufficiently, followed by adding bis(tri-tert-butylphosphine)palladium(0) (0.2 g, 0.3 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.3 g of Compound 1-6 (yield 76%, MS: [M+H]+=650).

### Preparation Example 1-7

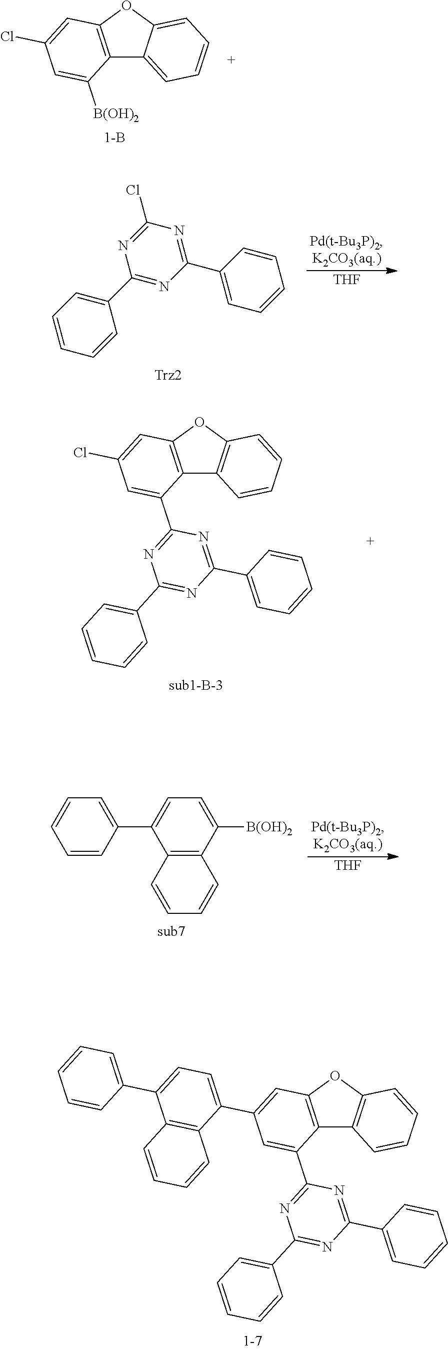

Compound 1-B (15 g, 60.9 mmol) and Trz2 (16.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 12 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.8 g of sub1-B-3 (yield 79%, MS: [M+H]+=434).

sub1-B-3 (15 g, 34.6 mmol) and sub7 (8.6 g, 34.6 mmol) were added to 300 ml of THF under a nitrogen atmosphere, and the mixture was stirred and refluxed. Then, potassium carbonate (9.6 g, 69.1 mmol) was dissolved in 29 ml of water, and then added thereto. Thereafter, it was stirred sufficiently, followed by adding bis(tri-tert-butylphosphine)palladium(0) (0.2 g, 0.3 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.4 g of Compound 1-7 (yield 74%, MS: [M+H]+=602).

### Preparation Example 1-8

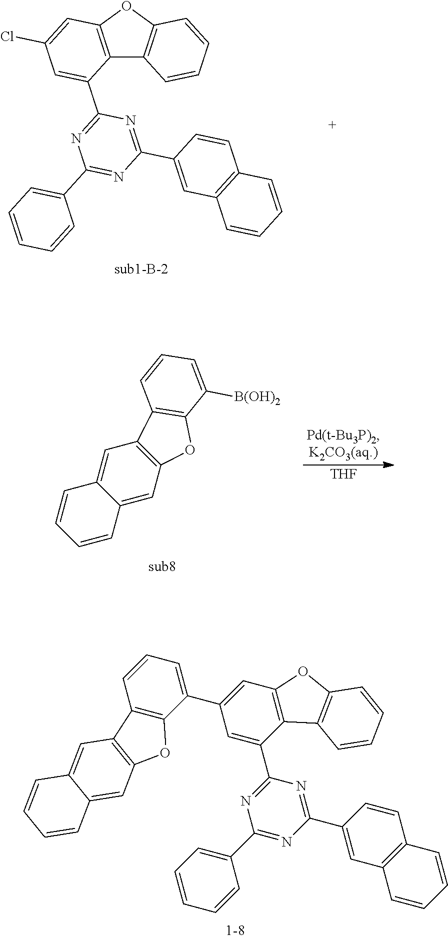 [SMILES: Clc1cc(-c2nc(-c3ccccc3)nc(-c3ccc4ccccc4c3)n2)c2c(c1)oc1ccccc12 ; OB(O)c1cccc2c1oc1cc3ccccc3cc12 ; c1ccc(-c2nc(-c3ccc4ccccc4c3)nc(-c3cc(-c4cccc5c4oc4cc6ccccc6cc45)cc4oc5ccccc5c34)n2)cc1 ; "THF"]

sub1-B-2 (15 g, 31 mmol) and sub8 (8.1 g, 31 mmol) were added to 300 ml of THF under a nitrogen atmosphere, and the mixture was stirred and refluxed. Then, potassium carbonate (8.6 g, 62 mmol) was dissolved in 26 ml of water, and then added thereto. Thereafter, it was stirred sufficiently, followed by adding bis(tri-tert-butylphosphine)palladium(0) (0.2 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.5 g of Compound 1-8 (yield 75%, MS: [M+H]+=666).

### Preparation Example 1-9

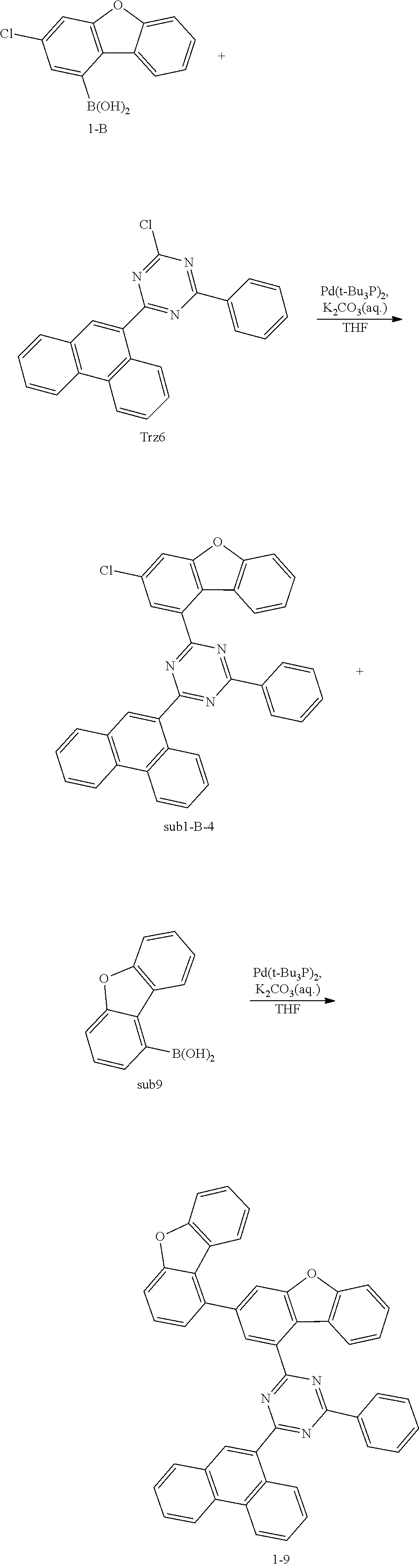

Compound 1-B (15 g, 60.9 mmol) and Trz6 (22.4 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23.7 g of sub1-B-4 (yield 73%, MS: [M+H]+=534).

sub1-B-4 (15 g, 28.1 mmol) and sub9 (6 g, 28.1 mmol) were added to 300 ml of THF under a nitrogen atmosphere, and the mixture was stirred and refluxed. Then, potassium carbonate (7.8 g, 56.2 mmol) was dissolved in 23 ml of water, and then added thereto. Thereafter, it was stirred sufficiently, followed by adding bis(tri-tert-butylphosphine)palladium(0) (0.1 g, 0.3 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.6 g of Compound 1-9 (yield 62%, MS: [M+H]+=666).

### Preparation Example 1-10

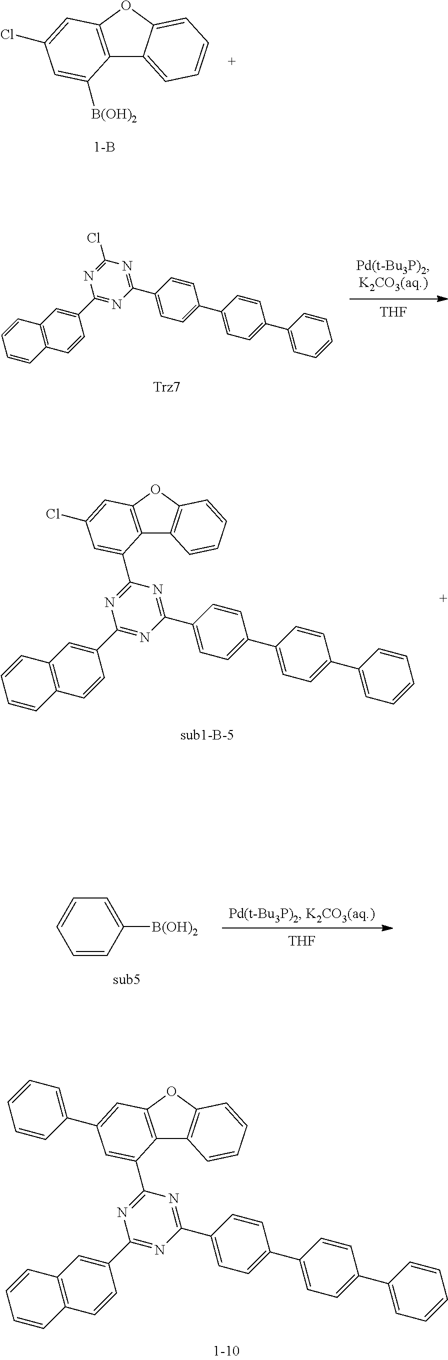

Compound 1-B (15 g, 60.9 mmol) and Trz7 (28.6 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 28.6 g of sub1-B-5 (yield 74%, MS: [M+H]+=636).

sub1-B-5 (15 g, 23.6 mmol) and sub5 (2.9 g, 23.6 mmol) were added to 300 ml of THF under a nitrogen atmosphere, and the mixture was stirred and refluxed. Then, potassium carbonate (6.5 g, 47.2 mmol) was dissolved in 20 ml of water, and then added thereto. Thereafter, it was stirred sufficiently, followed by adding bis(tri-tert-butylphosphine)palladium(0) (0.1 g, 0.2 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.4 g of Compound 1-5 (yield 65%, MS: [M+H]+=678).

### Preparation Example 1-11

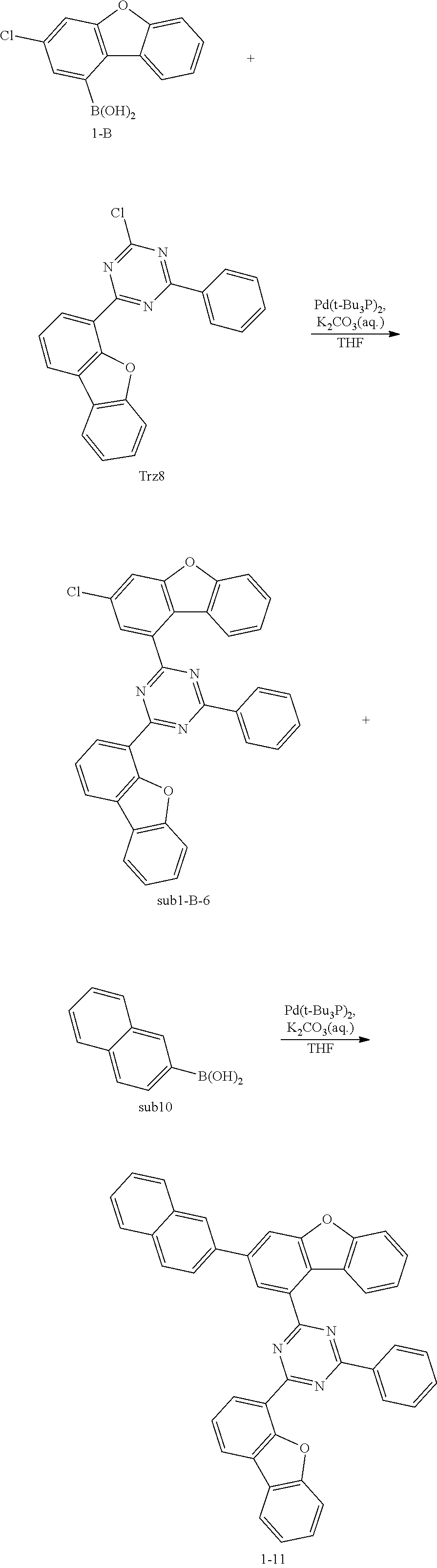

Compound 1-B (15 g, 60.9 mmol) and Trz8 (21.8 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.1 g of sub1-B-6 (yield 63%, MS: [M+H]+=524).

sub1-B-6 (15 g, 28.6 mmol) and sub10 (4.9 g, 28.6 mmol) were added to 300 ml of THF under a nitrogen atmosphere, and the mixture was stirred and refluxed. Then, potassium carbonate (7.9 g, 57.3 mmol) was dissolved in 24 ml of water, and then added thereto. Thereafter, it was stirred sufficiently, followed by adding bis(tri-tert-butylphosphine)palladium(0) (0.1 g, 0.3 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.4 g of Compound 1-11 (yield 65%, MS: [M+H]+=616).

### Preparation Example 1-12

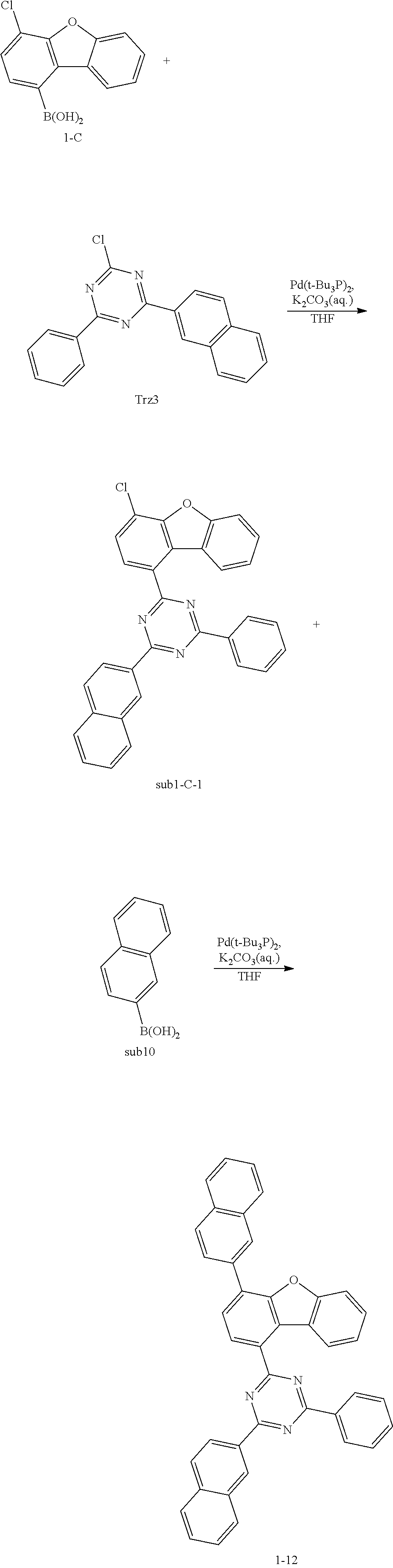

Compound 1-C (15 g, 60.9 mmol) and Trz3 (19.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 17.6 g of sub1-C-1 (yield 60%, MS: [M+H]+=484).

sub1-C-1 (15 g, 31 mmol) and sub10 (5.3 g, 31 mmol) were added to 300 ml of THF under a nitrogen atmosphere, and the mixture was stirred and refluxed. Then, potassium carbonate (8.6 g, 62 mmol) was dissolved in 26 ml of water, and then added thereto. Thereafter, it was stirred sufficiently, followed by adding bis(tri-tert-butylphosphine)palladium(0) (0.2 g, 0.3 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.8 g of Compound 1-12 (yield 72%, MS: [M+H]+=576).

### Preparation Example 1-13

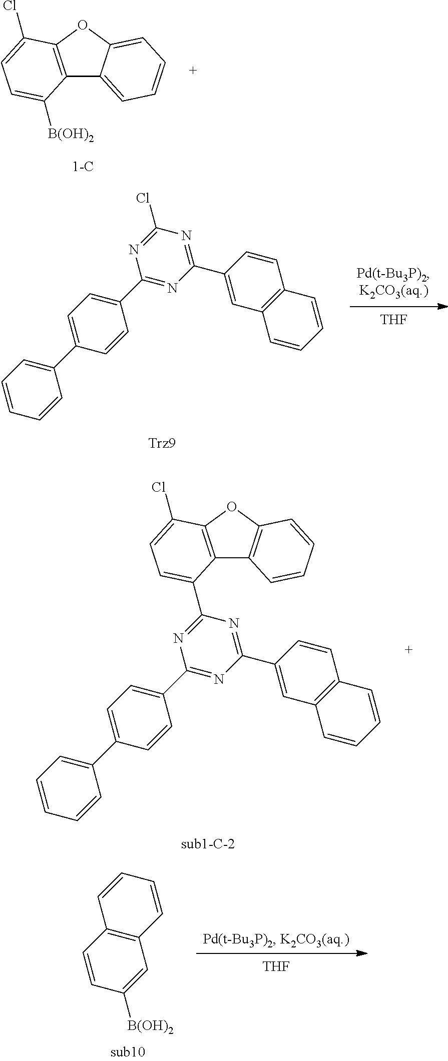 [SMILES: OB(O)c1ccc(Cl)c2oc3ccccc3c12 ; "THF" ; Clc1nc(-c2ccc(-c3ccccc3)cc2)nc(-c2ccc3ccccc3c2)n1 ; Clc1ccc(-c2nc(-c3ccc(-c4ccccc4)cc3)nc(-c3ccc4ccccc4c3)n2)c2c1oc1ccccc12 ; "THF" ; OB(O)c1ccc2ccccc2c1]

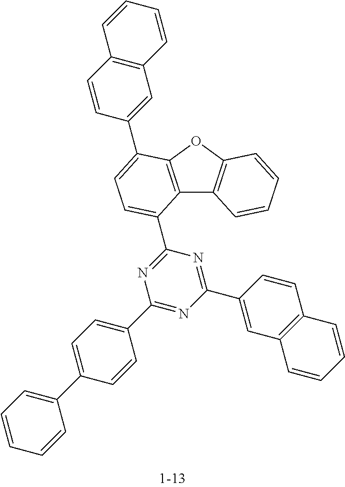 [SMILES: c1ccc(-c2ccc(-c3nc(-c4ccc5ccccc5c4)nc(-c4ccc(-c5ccc6ccccc6c5)c5oc6ccccc6c45)n3)cc2)cc1]

Compound 1-C (15 g, 60.9 mmol) and Trz9 (24 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 12 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23.5 g of sub1-C-2 (yield 69%, MS: [M+H]+=560).

sub1-C-2 (15 g, 26.8 mmol) and sub10 (4.6 g, 26.8 mmol) were added to 300 ml of THF under a nitrogen atmosphere, and the mixture was stirred and refluxed. Then, potassium carbonate (7.4 g, 53.6 mmol) was dissolved in 22 ml of water, and then added thereto. Thereafter, it was stirred sufficiently, followed by adding bis(tri-tert-butylphosphine)palladium(0) (0.1 g, 0.3 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14 g of Compound 1-13 (yield 80%, MS: [M+H]+=652).

### Preparation Example 1-14

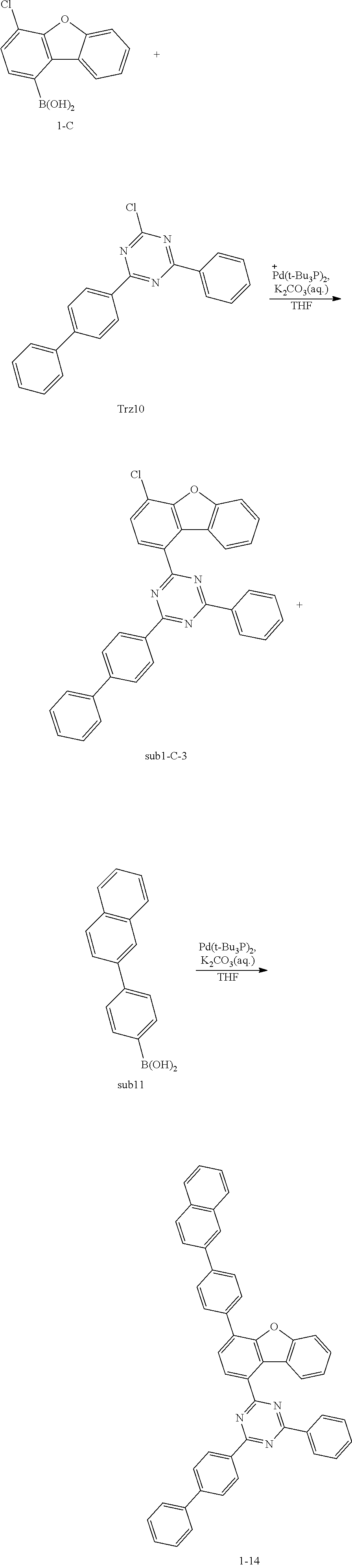

Compound 1-C (15 g, 60.9 mmol) and Trz10 (20.9 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.5 g of sub1-C-3 (yield 66%, MS: [M+H]+=510).

sub1-C-3 (15 g, 29.4 mmol) and sub11 (7.3 g, 29.4 mmol) were added to 300 ml of THF under a nitrogen atmosphere, and the mixture was stirred and refluxed. Then, potassium carbonate (8.1 g, 58.8 mmol) was dissolved in 24 ml of water, and then added thereto. Thereafter, it was stirred sufficiently, followed by adding bis(tri-tert-butylphosphine)palladium(0) (0.2 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.3 g of Compound 1-14 (yield 77%, MS: [M+H]+=678).

### Preparation Example 1-15

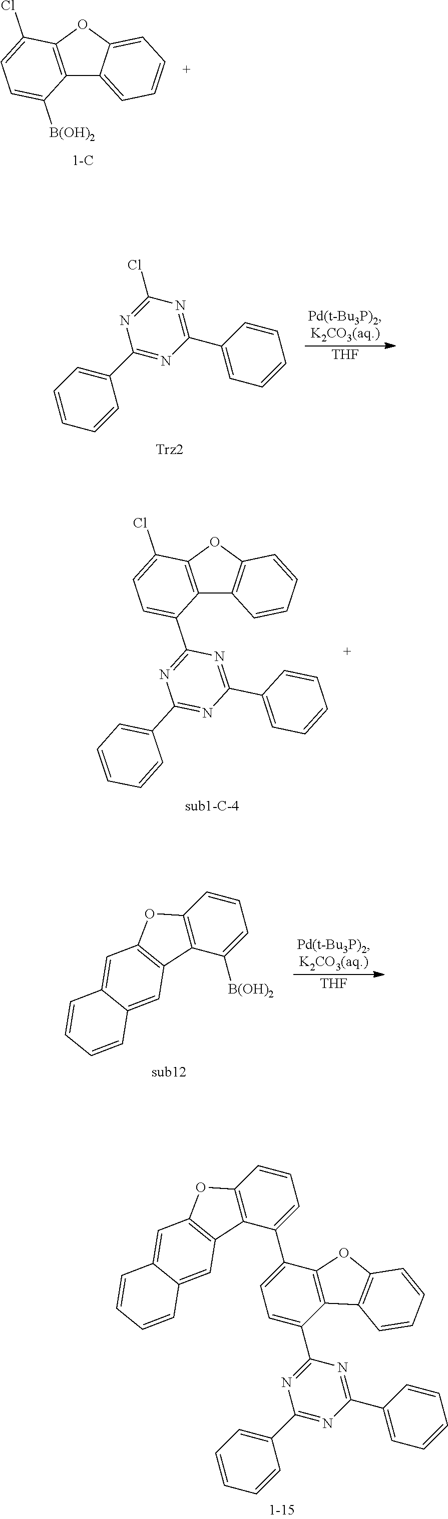

Compound 1-C (15 g, 60.9 mmol) and Trz2 (16.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 18.7 g of sub1-C-4 (yield 71%, MS: [M+H]+=434).

sub1-C-4 (15 g, 37.1 mmol) and sub12 (9.7 g, 37.1 mmol) were added to 300 ml of THF under a nitrogen atmosphere, and the mixture was stirred and refluxed. Then, potassium carbonate (10.3 g, 74.3 mmol) was dissolved in 31 ml of water, and then added thereto. Thereafter, it was stirred sufficiently, followed by adding bis(tri-tert-butylphosphine)palladium(0) (0.2 g, 0.4 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.6 g of Compound 1-15 (yield 64%, MS: [M+H]+=616).

### Preparation Example 1-16

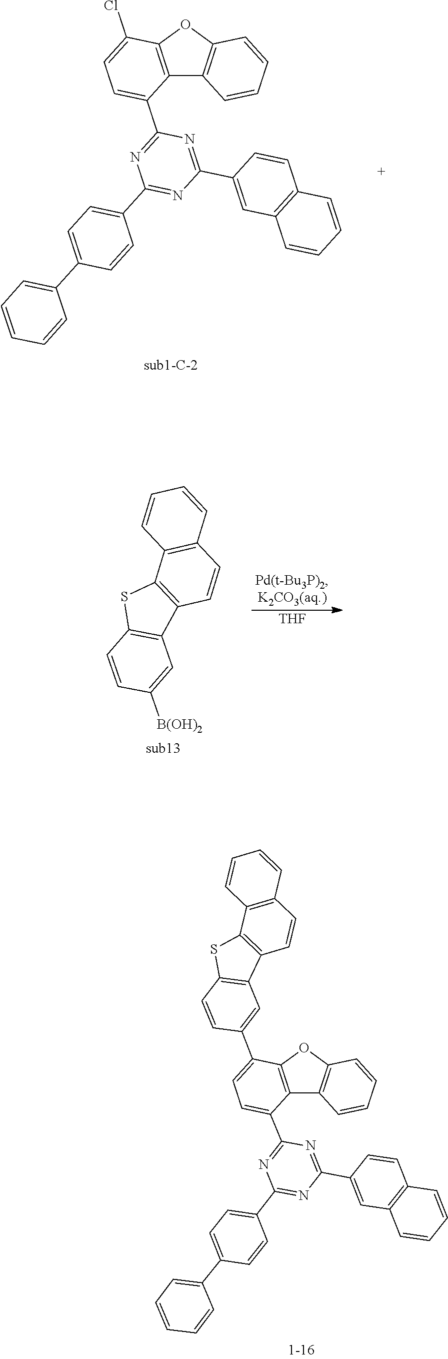 [SMILES: Clc1ccc(-c2nc(-c3ccc(-c4ccccc4)cc3)nc(-c3ccc4ccccc4c3)n2)c2c1oc1ccccc12 ; OB(O)c1ccc2sc3c4ccccc4ccc3c2c1 ; c1ccc(-c2ccc(-c3nc(-c4ccc5ccccc5c4)nc(-c4ccc(-c5ccc6sc7c8ccccc8ccc7c6c5)c5oc6ccccc6c45)n3)cc2)cc1 ; "THF"]

sub1-C-2 (15 g, 26.8 mmol) and sub13 (7.4 g, 26.8 mmol) were added to 300 ml of THF under a nitrogen atmosphere, and the mixture was stirred and refluxed. Then, potassium carbonate (7.4 g, 53.6 mmol) was dissolved in 22 ml of water, and then added thereto. Thereafter, it was stirred sufficiently, followed by adding bis(tri-tert-butylphosphine)palladium(0) (0.1 g, 0.3 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 16.2 g of Compound 1-16 (yield 80%, MS: [M+H]+=758).

### Preparation Example 1-17

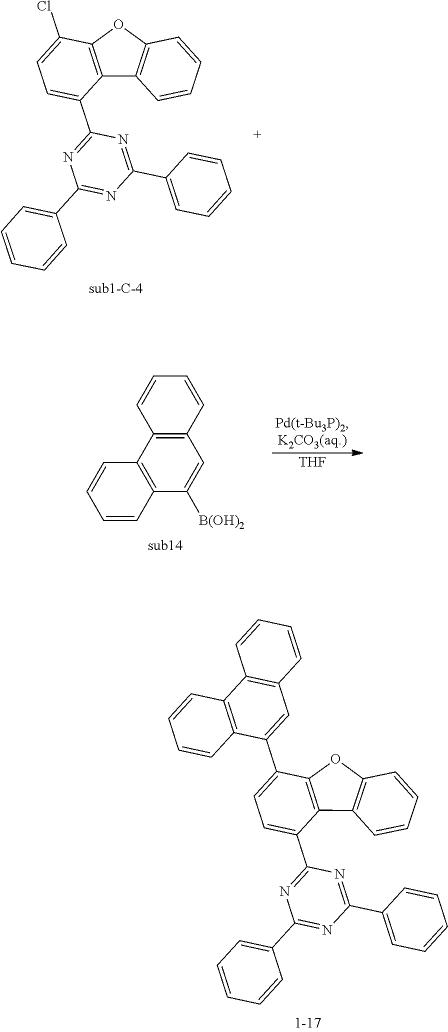 [SMILES: Clc1ccc(-c2nc(-c3ccccc3)nc(-c3ccccc3)n2)c2c1oc1ccccc12 ; OB(O)c1cc2ccccc2c2ccccc12 ; c1ccc(-c2nc(-c3ccccc3)nc(-c3ccc(-c4cc5ccccc5c5ccccc45)c4oc5ccccc5c34)n2)cc1 ; "THF"]

sub1-C-4 (15 g, 34.6 mmol) and sub14 (7.7 g, 34.6 mmol) were added to 300 ml of THF under a nitrogen atmosphere, and the mixture was stirred and refluxed. Then, potassium carbonate (9.6 g, 69.1 mmol) was dissolved in 29 ml of water, and then added thereto. Thereafter, it was stirred sufficiently, followed by adding bis(tri-tert-butylphosphine)palladium(0) (0.2 g, 0.3 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.3 g of Compound 1-17 (yield 62%, MS: [M+H]+=576).

### Preparation Example 1-18

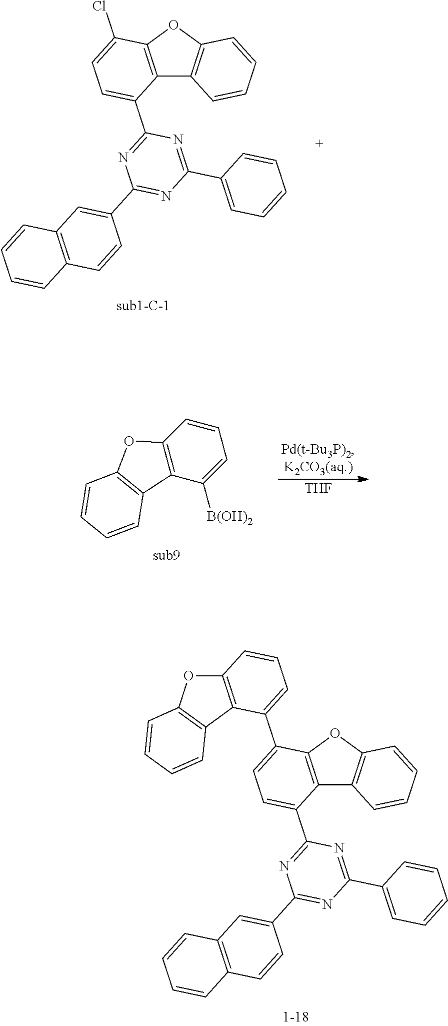 [SMILES: Clc1ccc(-c2nc(-c3ccccc3)nc(-c3ccc4ccccc4c3)n2)c2c1oc1ccccc12 ; OB(O)c1cccc2oc3ccccc3c12 ; c1ccc(-c2nc(-c3ccc4ccccc4c3)nc(-c3ccc(-c4cccc5oc6ccccc6c45)c4oc5ccccc5c34)n2)cc1 ; "THF"]

sub1-C-1 (15 g, 31 mmol) and sub9 (6.6 g, 31 mmol) were added to 300 ml of THF under a nitrogen atmosphere, and the mixture was stirred and refluxed. Then, potassium carbonate (8.6 g, 62 mmol) was dissolved in 26 ml of water, and then added thereto. Thereafter, it was stirred sufficiently, followed by adding bis(tri-tert-butylphosphine)palladium(0) (0.2 g, 0.3 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12 g of Compound 1-18 (yield 63%, MS: [M+H]+=616).

### Preparation Example 1-19

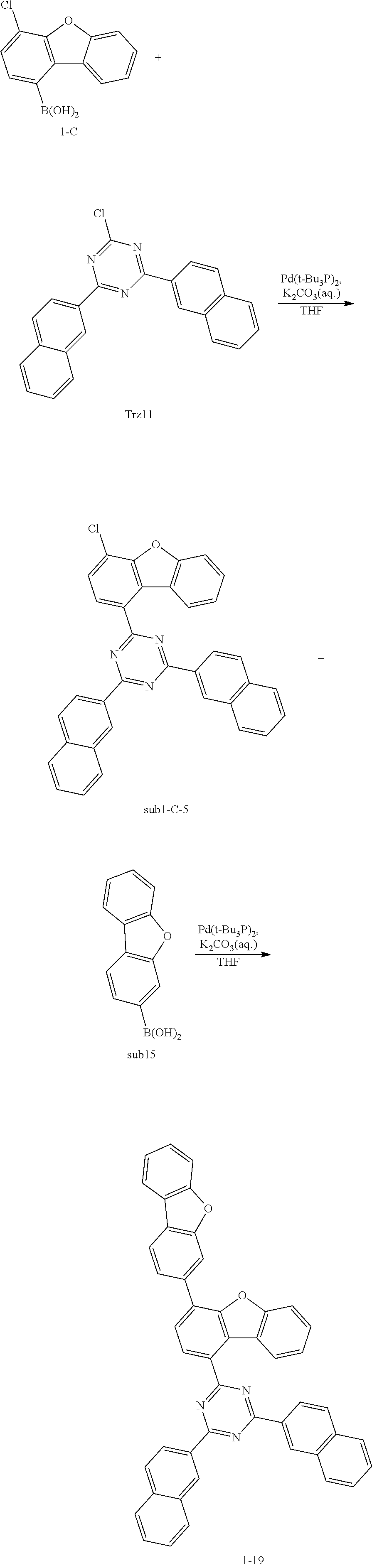

Compound 1-C (15 g, 60.9 mmol) and Trz11 (22.4 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.4 g of sub1-C-5 (yield 69%, MS: [M+H]+=534).

sub1-C-5 (15 g, 28.1 mmol) and sub15 (6 g, 28.1 mmol) were added to 300 ml of THF under a nitrogen atmosphere, and the mixture was stirred and refluxed. Then, potassium carbonate (7.8 g, 56.2 mmol) was dissolved in 23 ml of water, and then added thereto. Thereafter, it was stirred sufficiently, followed by adding bis(tri-tert-butylphosphine)palladium(0) (0.1 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.3 g of Compound 1-19 (yield 71%, MS: [M+H]+=666).

### Preparation Example 1-20

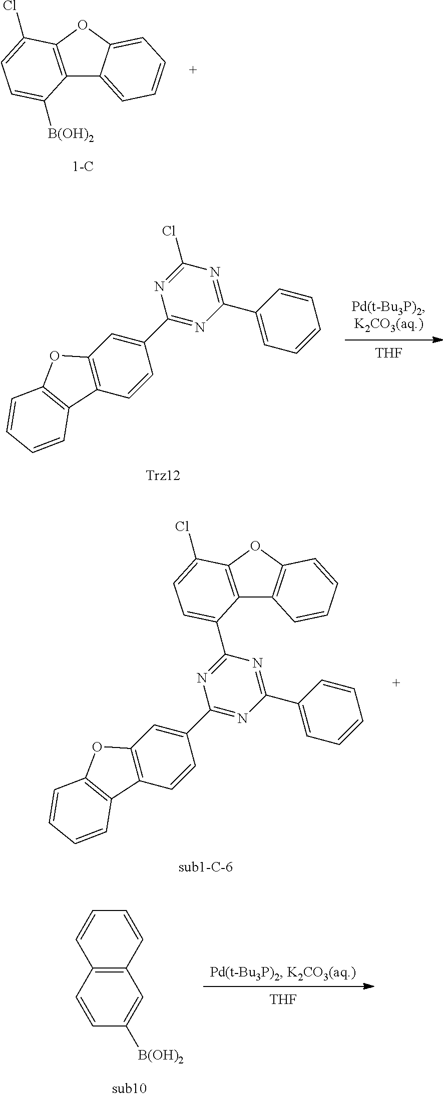 [SMILES: OB(O)c1ccc(Cl)c2oc3ccccc3c12 ; "THF" ; Clc1nc(-c2ccccc2)nc(-c2ccc3c(c2)oc2ccccc23)n1 ; Clc1ccc(-c2nc(-c3ccccc3)nc(-c3ccc4c(c3)oc3ccccc34)n2)c2c1oc1ccccc12 ; "THF" ; OB(O)c1ccc2ccccc2c1]

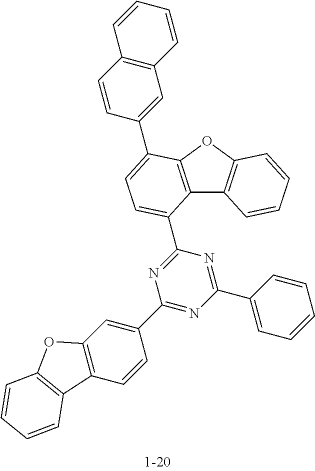 [SMILES: c1ccc(-c2nc(-c3ccc4c(c3)oc3ccccc34)nc(-c3ccc(-c4ccc5ccccc5c4)c4oc5ccccc5c34)n2)cc1]

Compound 1-C (15 g, 60.9 mmol) and Trz12 (21.8 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21 g of sub1-C-6 (yield 66%, MS: [M+H]+=524).

sub1-C-6 (15 g, 28.6 mmol) and sub10 (4.9 g, 28.6 mmol) were added to 300 ml of THF under a nitrogen atmosphere, and the mixture was stirred and refluxed. Then, potassium carbonate (11.9 g, 85.9 mmol) was dissolved in 36 ml of water, and then added thereto. Thereafter, it was stirred sufficiently, followed by adding bis(tri-tert-butylphosphine)palladium(0) (0.1 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.3 g of Compound 1-20 (yield 70%, MS: [M+H]+=616).

### Preparation Example 1-21

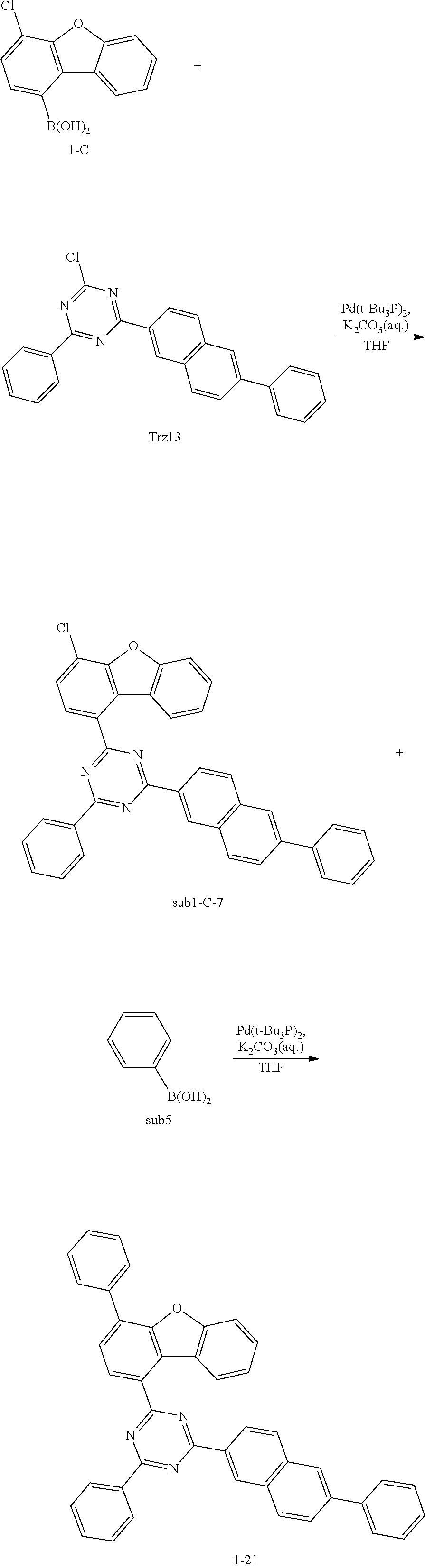

Compound 1-C (15 g, 60.9 mmol) and Trz13 (24 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 12 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 26.2 g of sub1-C-7 (yield 77%, MS: [M+H]+=560).

sub1-C-7 (15 g, 26.8 mmol) and sub5 (3.3 g, 26.8 mmol) were added to 300 ml of THF under a nitrogen atmosphere, and the mixture was stirred and refluxed. Then, potassium carbonate (11.1 g, 80.3 mmol) was dissolved in 33 ml of water, and then added thereto. Thereafter, it was stirred sufficiently, followed by adding bis(tri-tert-butylphosphine)palladium(0) (0.1 g, 0.3 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.5 g of Compound 1-21 (yield 65%, MS: [M+H]+=602).

### Preparation Example 1-22

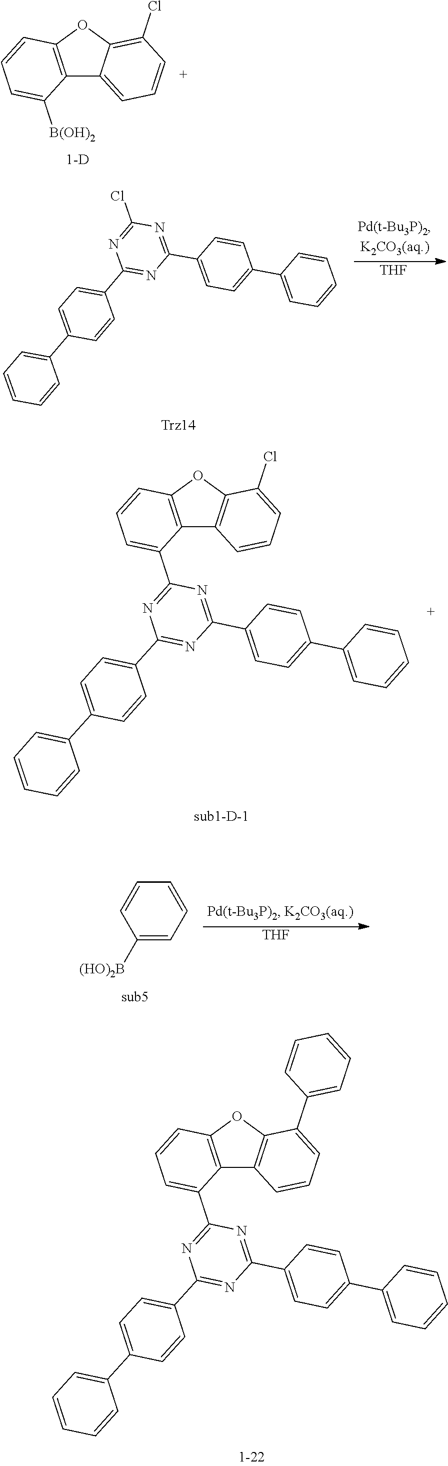

Compound 1-D (15 g, 60.9 mmol) and Trz14 (19.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23.9 g of sub1-D-1 (yield 67%, MS: [M+H]+=586).

sub1-D-1 (15 g, 25.6 mmol) and sub5 (3.1 g, 25.6 mmol) were added to 300 ml of THF under a nitrogen atmosphere, and the mixture was stirred and refluxed. Then, potassium carbonate (10.6 g, 76.8 mmol) was dissolved in 32 ml of water, and then added thereto. Thereafter, it was stirred sufficiently, followed by adding bis(tri-tert-butylphosphine)palladium(0) (0.1 g, 0.3 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.3 g of Compound 1-22 (yield 64%, MS: [M+H]+=628).

### Preparation Example 1-23

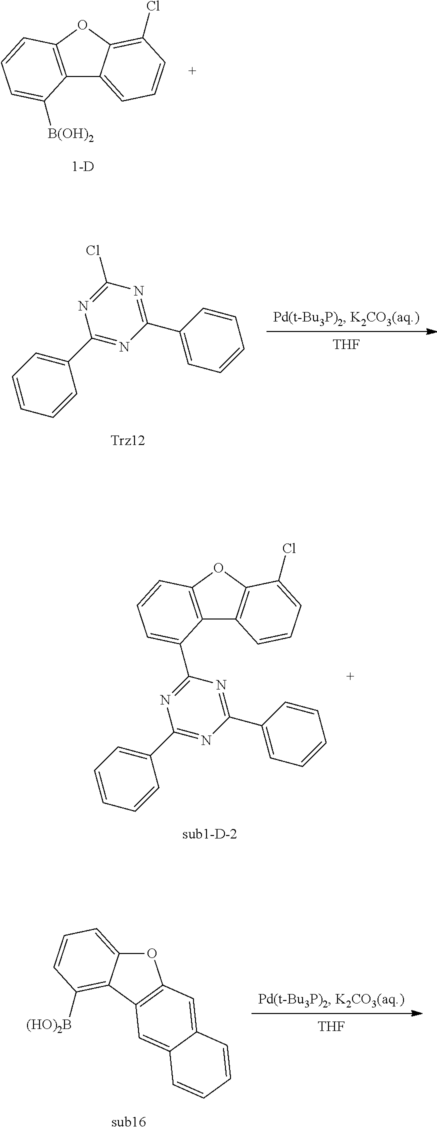 [SMILES: OB(O)c1cccc2oc3c(Cl)cccc3c12 ; "THF" ; Clc1nc(-c2ccccc2)nc(-c2ccccc2)n1 ; Clc1cccc2c1oc1cccc(-c3nc(-c4ccccc4)nc(-c4ccccc4)n3)c12 ; "THF" ; OB(O)c1cccc2oc3cc4ccccc4cc3c12]

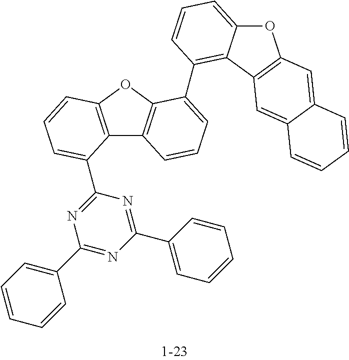 [SMILES: c1ccc(-c2nc(-c3ccccc3)nc(-c3cccc4oc5c(-c6cccc7oc8cc9ccccc9cc8c67)cccc5c34)n2)cc1]

Compound 1-D (15 g, 60.9 mmol) and Trz2 (16.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20 g of sub1-D-2 (yield 76%, MS: [M+H]+=434).

sub1-D-2 (15 g, 34.6 mmol) and sub16 (9.1 g, 34.6 mmol) were added to 300 ml of THF under a nitrogen atmosphere, and the mixture was stirred and refluxed. Then, potassium carbonate (14.3 g, 103.7 mmol) was dissolved in 43 ml of water, and then added thereto. Thereafter, it was stirred sufficiently, followed by adding bis(tri-tert-butylphosphine)palladium(0) (0.2 g, 0.3 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14 g of Compound 1-23 (yield 66%, MS: [M+H]+=616).

### Preparation Example 1-24

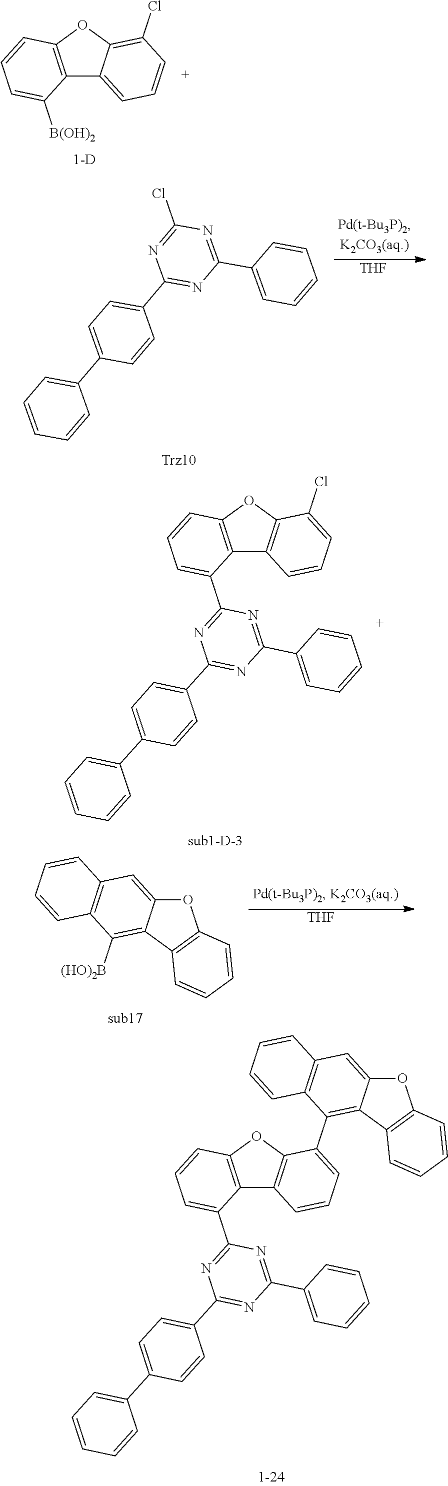

Compound 1-D (15 g, 60.9 mmol) and Trz10 (20.9 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 12 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.8 g of sub1-D-3 (yield 67%, MS: [M+H]+=510).

sub1-D-3 (15 g, 29.4 mmol) and sub17 (7.7 g, 29.4 mmol) were added to 300 ml of THF under a nitrogen atmosphere, and the mixture was stirred and refluxed. Then, potassium carbonate (12.2 g, 88.2 mmol) was dissolved in 37 ml of water, and then added thereto. Thereafter, it was stirred sufficiently, followed by adding bis(tri-tert-butylphosphine)palladium(0) (0.2 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.4 g of Compound 1-24 (yield 61%, MS: [M+H]+=692).

### Preparation Example 1-25

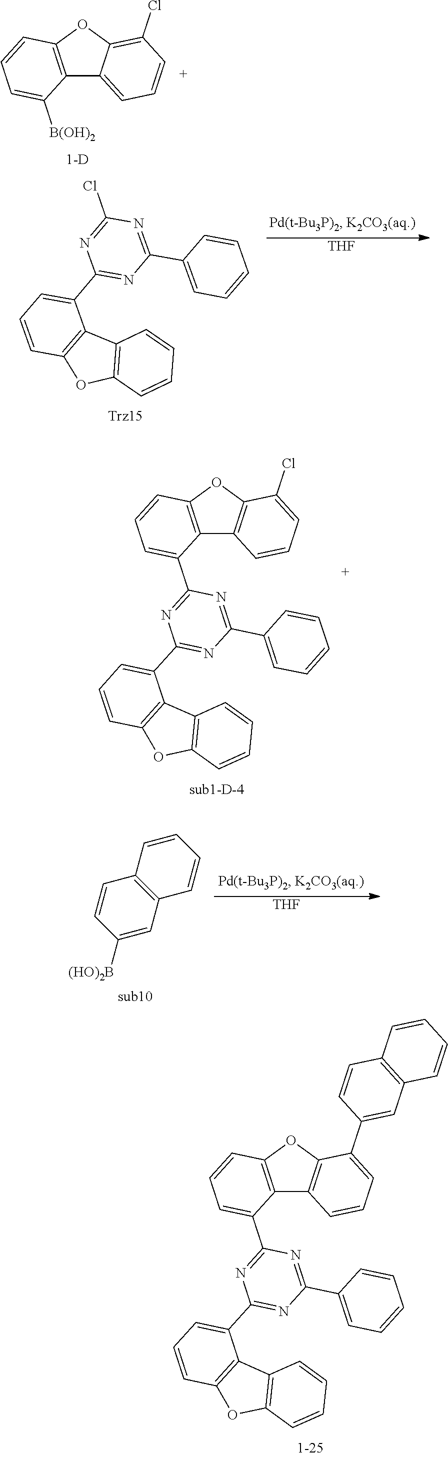

Compound 1-D (15 g, 60.9 mmol) and Trz15 (21.8 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21.3 g of sub1-D-4 (yield 67%, MS: [M+H]+=524).

sub1-D-4 (15 g, 28.6 mmol) and sub10 (4.9 g, 28.6 mmol) were added to 300 ml of THF under a nitrogen atmosphere, and the mixture was stirred and refluxed. Then, potassium carbonate (11.9 g, 85.9 mmol) was dissolved in 36 ml of water, and then added thereto. Thereafter, it was stirred sufficiently, followed by adding bis(tri-tert-butylphosphine)palladium(0) (0.1 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.7 g of Compound 1-25 (yield 61%, MS: [M+H]+=616).

### Preparation Example 1-26

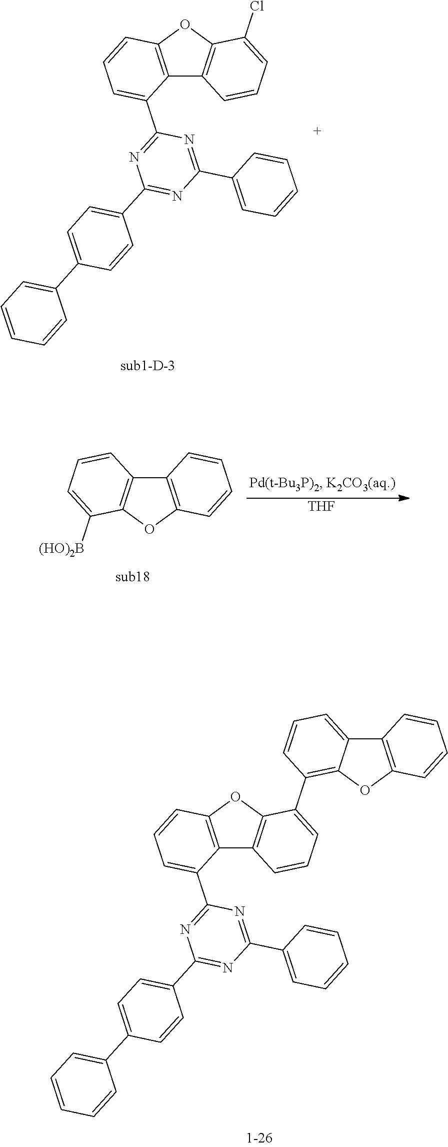 [SMILES: Clc1cccc2c1oc1cccc(-c3nc(-c4ccccc4)nc(-c4ccc(-c5ccccc5)cc4)n3)c12 ; OB(O)c1cccc2c1oc1ccccc12 ; c1ccc(-c2ccc(-c3nc(-c4ccccc4)nc(-c4cccc5oc6c(-c7cccc8c7oc7ccccc78)cccc6c45)n3)cc2)cc1 ; "THF"]

sub1-D-3 (15 g, 29.4 mmol) and sub18 (6.2 g, 29.4 mmol) were added to 300 ml of THF under a nitrogen atmosphere, and the mixture was stirred and refluxed. Then, potassium carbonate (12.2 g, 88.2 mmol) was dissolved in 37 ml of water, and then added thereto. Thereafter, it was stirred sufficiently, followed by adding bis(tri-tert-butylphosphine)palladium(0) (0.2 g, 0.3 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.3 g of Compound 1-26 (yield 76%, MS: [M+H]+=642).

### Preparation Example 1-27

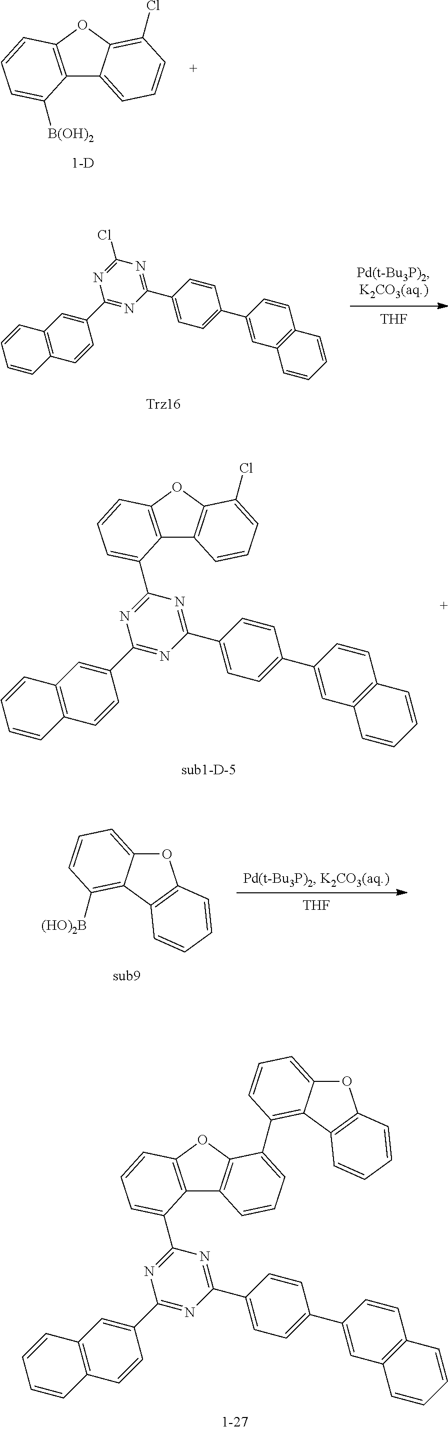

Compound 1-D (15 g, 60.9 mmol) and Trz16 (27 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 27.1 g of sub1-D-5 (yield 73%, MS: [M+H]+=610).

sub1-D-5 (15 g, 24.6 mmol) and sub9 (5.2 g, 24.6 mmol) were added to 300 ml of THF under a nitrogen atmosphere, and the mixture was stirred and refluxed. Then, potassium carbonate (10.2 g, 73.8 mmol) was dissolved in 31 ml of water, and then added thereto. Thereafter, it was stirred sufficiently, followed by adding bis(tri-tert-butylphosphine)palladium(0) (0.1 g, 0.2 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.8 g of Compound 1-27 (yield 70%, MS: [M+H]+=742).

### Preparation Example 1-28

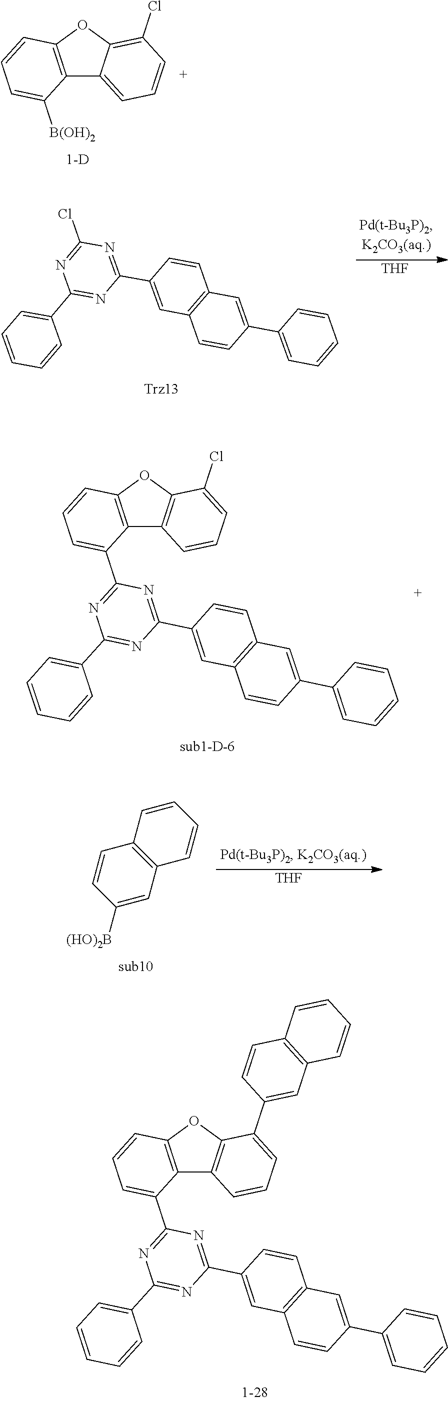

Compound 1-D (15 g, 60.9 mmol) and Trz13 (24 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.8 g of sub1-D-6 (yield 61%, MS: [M+H]+=560).

sub1-D-6 (15 g, 26.8 mmol) and sub10 (4.6 g, 26.8 mmol) were added to 300 ml of THF under a nitrogen atmosphere, and the mixture was stirred and refluxed. Then, potassium carbonate (11.1 g, 80.3 mmol) was dissolved in 33 ml of water, and then added thereto. Thereafter, it was stirred sufficiently, followed by adding bis(tri-tert-butylphosphine)palladium(0) (0.1 g, 0.3 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.2 g of Compound 1-28 (yield 70%, MS: [M+H]+=652).

### Preparation Example 1-29

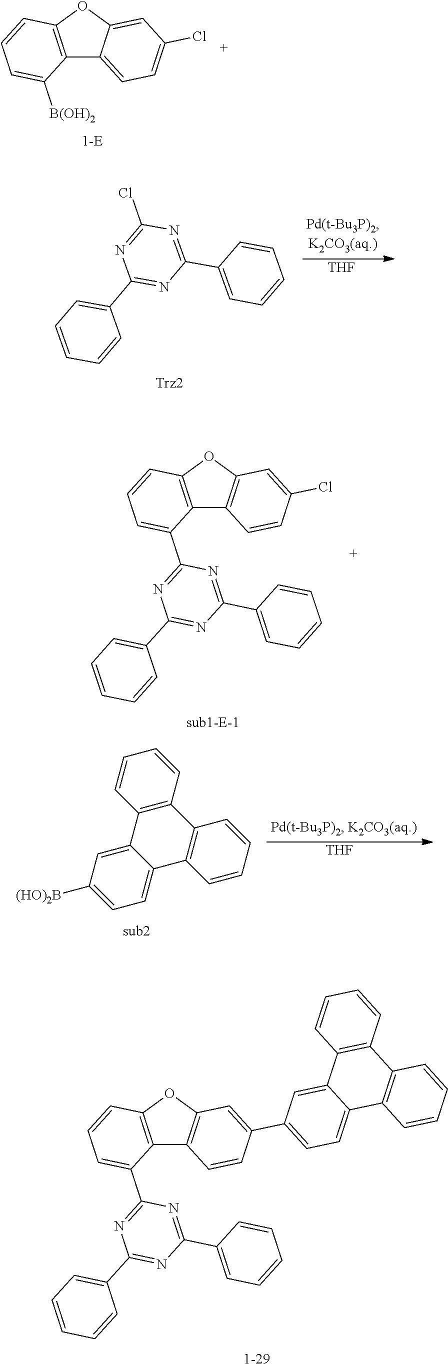

Compound 1-E (15 g, 60.9 mmol) and Trz2 (16.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 17.1 g of sub1-E-1 (yield 65%, MS: [M+H]+=434).

sub1-E-1 (15 g, 34.6 mmol) and sub2 (9.4 g, 34.6 mmol) were added to 300 ml of THF under a nitrogen atmosphere, and the mixture was stirred and refluxed. Then, potassium carbonate (14.3 g, 103.7 mmol) was dissolved in 43 ml of water, and then added thereto. Thereafter, it was stirred sufficiently, followed by adding bis(tri-tert-butylphosphine)palladium(0) (0.2 g, 0.3 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.5 g of Compound 1-29 (yield 67%, MS: [M+H]+=626).

### Preparation Example 1-30

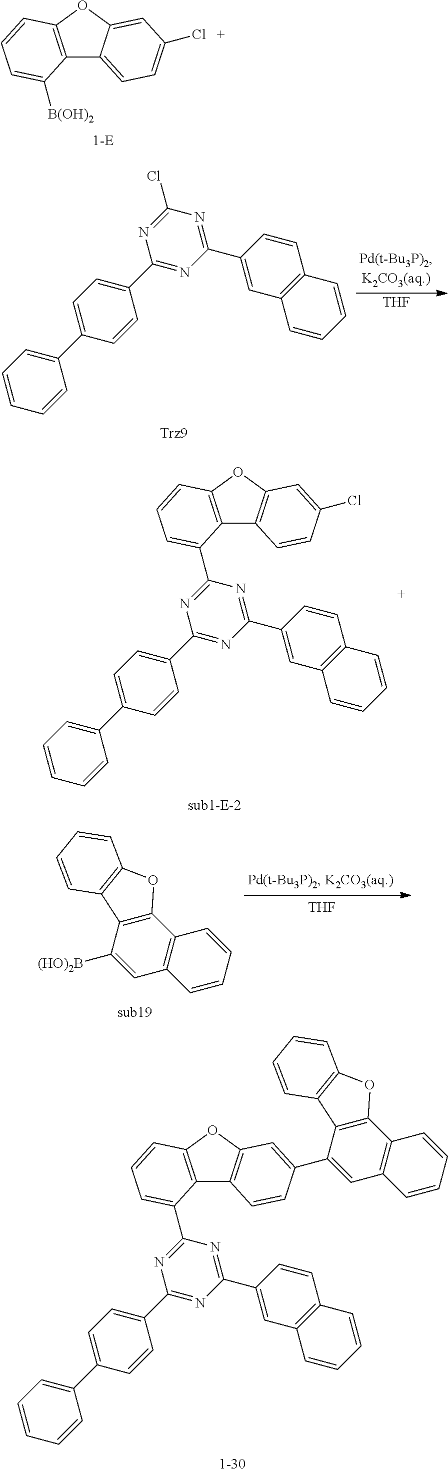

Compound 1-E (15 g, 60.9 mmol) and Trz9 (24 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 26.9 g of sub1-E-2 (yield 79%, MS: [M+H]+=560).

sub1-E-2 (15 g, 26.8 mmol) and sub19 (7 g, 26.8 mmol) were added to 300 ml of THF under a nitrogen atmosphere, and the mixture was stirred and refluxed. Then, potassium carbonate (11.1 g, 80.3 mmol) was dissolved in 33 ml of water, and then added thereto. Thereafter, it was stirred sufficiently, followed by adding bis(tri-tert-butylphosphine)palladium(0) (0.1 g, 0.3 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.9 g of Compound 1-30 (yield 80%, MS: [M+H]+=742).

### Preparation Example 1-31

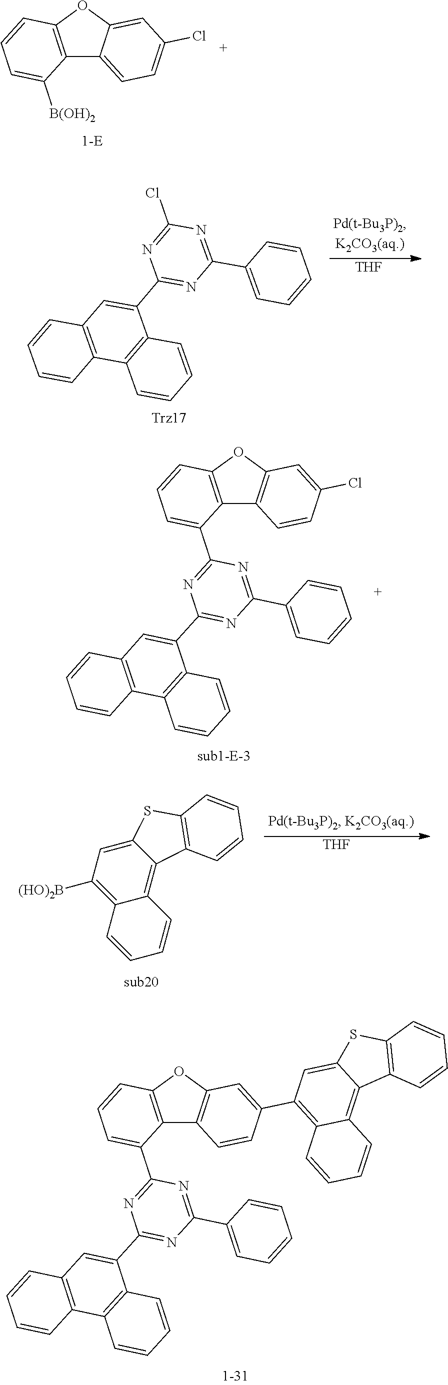

Compound 1-E (15 g, 60.9 mmol) and Trz17 (22.4 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 25.3 g of sub1-E-3 (yield 78%, MS: [M+H]+=534).

sub1-E-3 (15 g, 28.1 mmol) and sub20 (7.8 g, 28.1 mmol) were added to 300 ml of THF under a nitrogen atmosphere, and the mixture was stirred and refluxed. Then, potassium carbonate (11.6 g, 84.3 mmol) was dissolved in 35 ml of water, and then added thereto. Thereafter, it was stirred sufficiently, followed by adding bis(tri-tert-butylphosphine)palladium(0) (0.1 g, 0.3 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.8 g of Compound 1-31 (yield 72%, MS: [M+H]+=732).

### Preparation Example 1-32

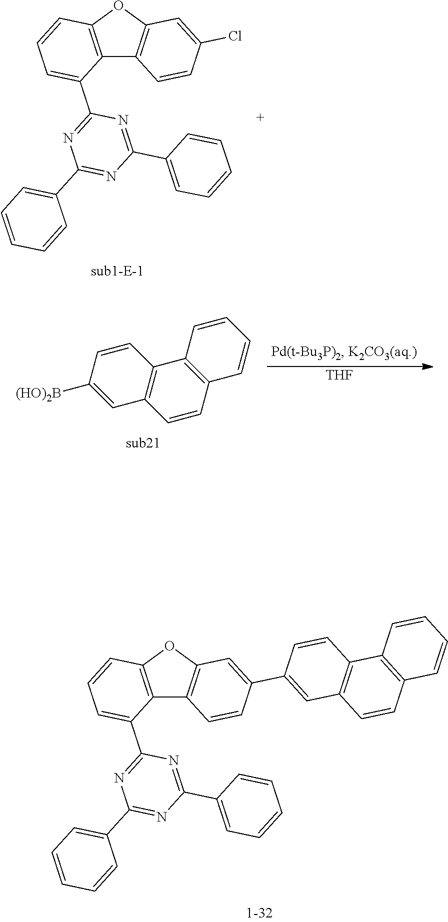 [SMILES: Clc1ccc2c(c1)oc1cccc(-c3nc(-c4ccccc4)nc(-c4ccccc4)n3)c12 ; "THF" ; OB(O)c1ccc2c(ccc3ccccc32)c1 ; c1ccc(-c2nc(-c3ccccc3)nc(-c3cccc4oc5cc(-c6ccc7c(ccc8ccccc87)c6)ccc5c34)n2)cc1]

sub1-E-1 (15 g, 34.6 mmol) and sub21 (7.7 g, 34.6 mmol) were added to 300 ml of THF under a nitrogen atmosphere, and the mixture was stirred and refluxed. Then, potassium carbonate (14.3 g, 103.7 mmol) was dissolved in 43 ml of water, and then added thereto. Thereafter, it was stirred sufficiently, followed by adding bis(tri-tert-butylphosphine)palladium(0) (0.2 g, 0.3 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.9 g of Compound 1-32 (yield 65%, MS: [M+H]+=576).

### Preparation Example 1-33

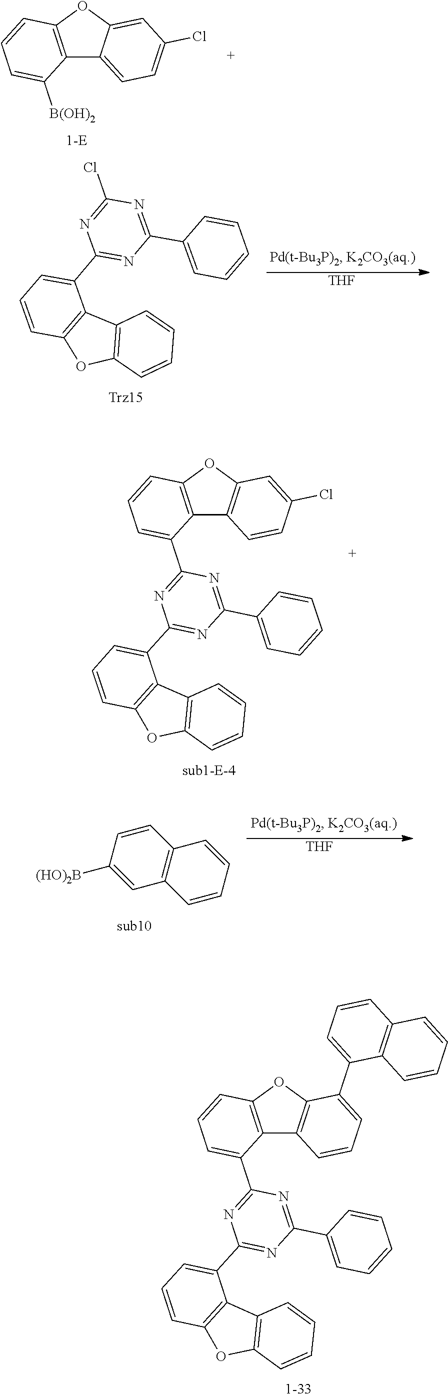

Compound 1-E (15 g, 60.9 mmol) and Trz15 (21.8 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 25.5 g of sub1-E-4 (yield 80%, MS: [M+H]+=524).

sub1-E-4 (15 g, 28.6 mmol) and sub10 (4.9 g, 28.6 mmol) were added to 300 ml of THF under a nitrogen atmosphere, and the mixture was stirred and refluxed. Then, potassium carbonate (11.9 g, 85.9 mmol) was dissolved in 36 ml of water, and then added thereto. Thereafter, it was stirred sufficiently, followed by adding bis(tri-tert-butylphosphine)palladium(0) (0.1 g, 0.3 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. [570] The concentrated compound was purified by silica gel column chromatography to prepare 10.6 g of Compound 1-33 (yield 60%, MS: [M+H]+=616).

### Preparation Example 1-34

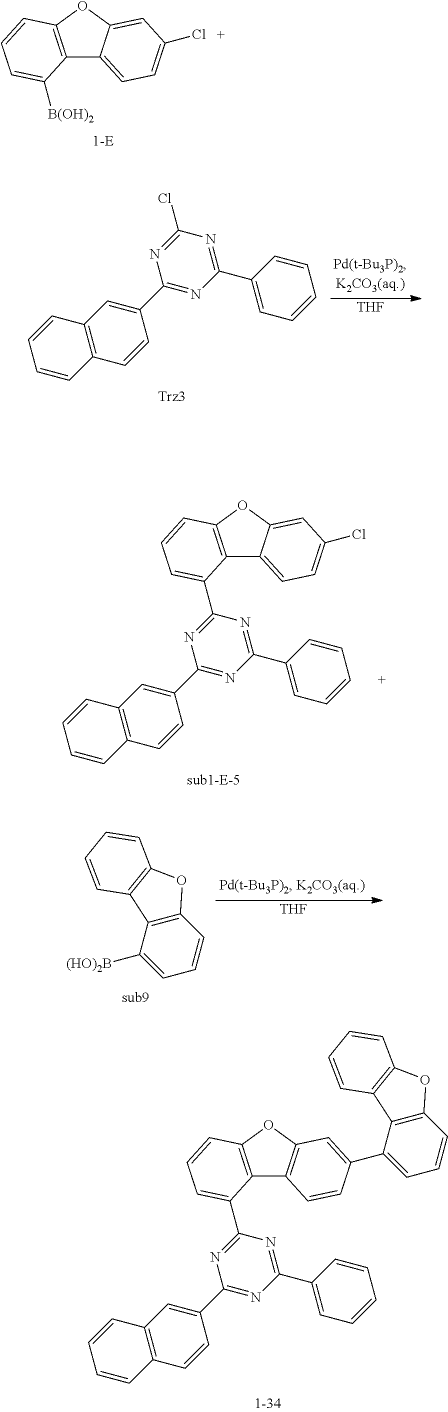

Compound 1-E (15 g, 60.9 mmol) and Trz3 (19.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 17.6 g of sub1-E-5 (yield 60%, MS: [M+H]+=484).

sub1-E-5 (15 g, 31 mmol) and sub9 (6.6 g, 31 mmol) were added to 300 ml of THF under a nitrogen atmosphere, and the mixture was stirred and refluxed. Then, potassium carbonate (12.9 g, 93 mmol) was dissolved in 39 ml of water, and then added thereto. Thereafter, it was stirred sufficiently, followed by adding bis(tri-tert-butylphosphine)palladium(0) (0.2 g, 0.3 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.4 g of Compound 1-34 (yield 60%, MS: [M+H]+=616).

### Preparation Example 1-35

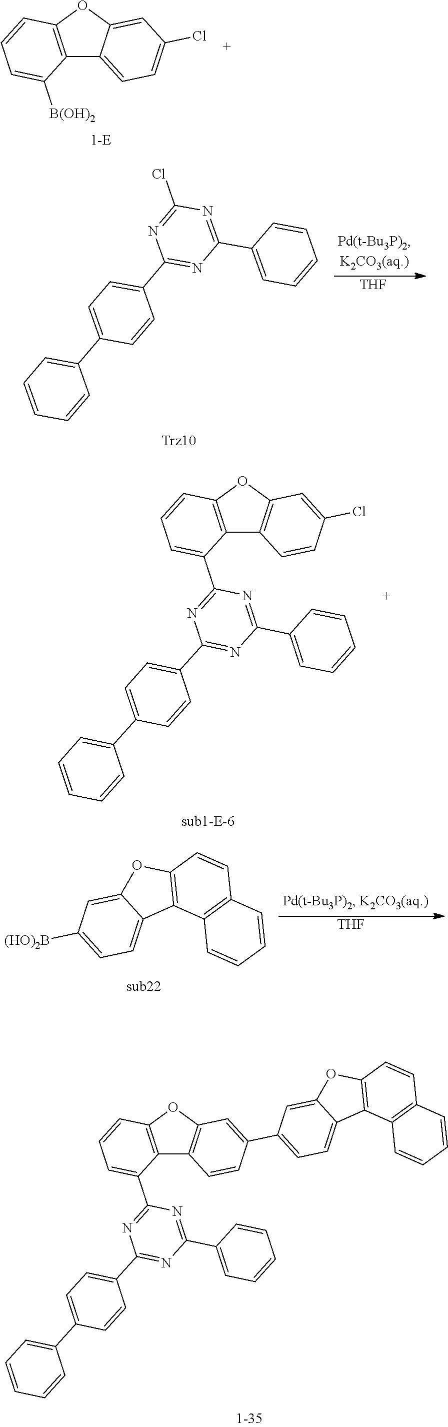

Compound 1-E (15 g, 60.9 mmol) and Trz10 (20.9 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21.7 g of sub1-E-6 (yield 70%, MS: [M+H]+=510).

sub1-E-6 (15 g, 29.4 mmol) and sub22 (7.7 g, 29.4 mmol) were added to 300 ml of THF under a nitrogen atmosphere, and the mixture was stirred and refluxed. Then, potassium carbonate (12.2 g, 88.2 mmol) was dissolved in 37 ml of water, and then added thereto. Thereafter, it was stirred sufficiently, followed by adding bis(tri-tert-butylphosphine)palladium(0) (0.2 g, 0.3 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.6 g of Compound 1-35 (yield 72%, MS: [M+H]+=692).

### Preparation Example 1-36

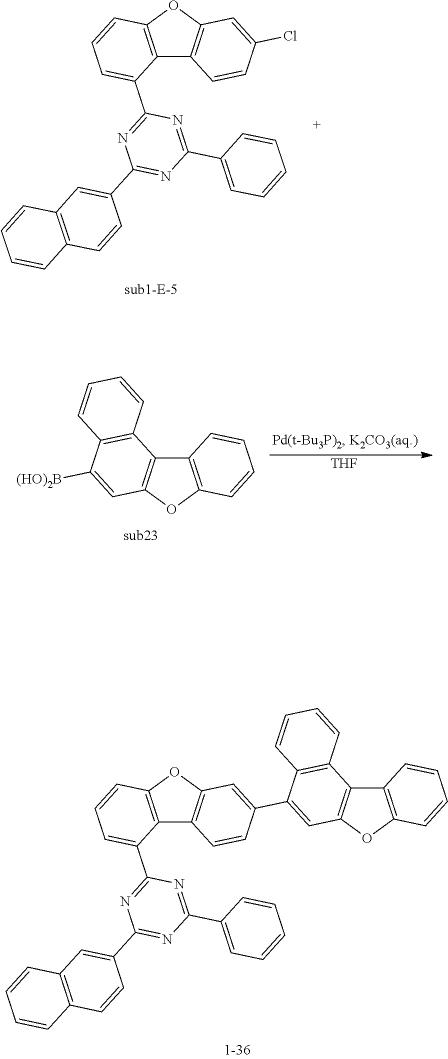 [SMILES: Clc1ccc2c(c1)oc1cccc(-c3nc(-c4ccccc4)nc(-c4ccc5ccccc5c4)n3)c12 ; OB(O)c1cc2oc3ccccc3c2c2ccccc12 ; c1ccc(-c2nc(-c3ccc4ccccc4c3)nc(-c3cccc4oc5cc(-c6cc7oc8ccccc8c7c7ccccc67)ccc5c34)n2)cc1 ; "THF"]

sub1-E-5 (15 g, 31 mmol) and sub23 (8.1 g, 31 mmol) were added to 300 ml of THF under a nitrogen atmosphere, and the mixture was stirred and refluxed. Then, potassium carbonate (12.9 g, 93 mmol) was dissolved in 39 ml of water, and then added thereto. Thereafter, it was stirred sufficiently, followed by adding bis(tri-tert-butylphosphine)palladium(0) (0.2 g, 0.3 mmol). After 12 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.4 g of Compound 1-36 (yield 60%, MS: [M+H]+=666).

### Preparation Example 1-37

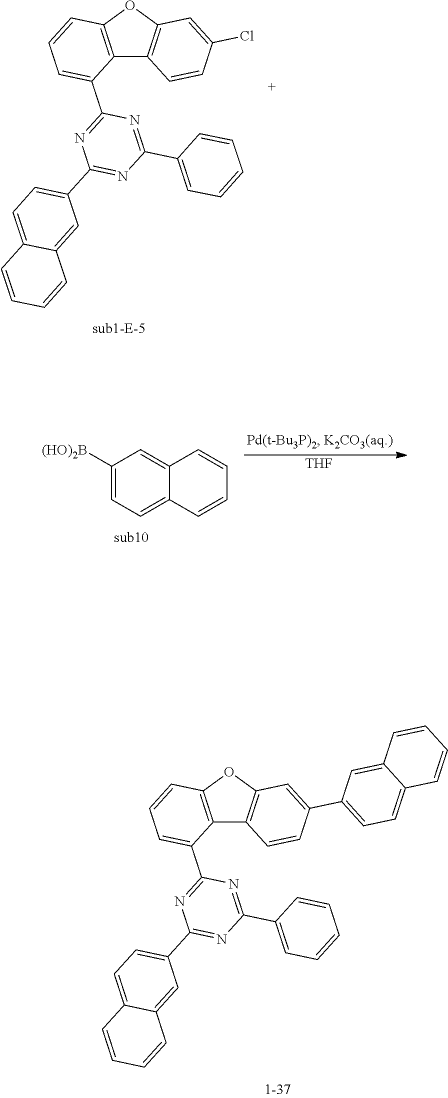 [SMILES: Clc1ccc2c(c1)oc1cccc(-c3nc(-c4ccccc4)nc(-c4ccc5ccccc5c4)n3)c12 ; OB(O)c1ccc2ccccc2c1 ; c1ccc(-c2nc(-c3ccc4ccccc4c3)nc(-c3cccc4oc5cc(-c6ccc7ccccc7c6)ccc5c34)n2)cc1 ; "THF"]

sub1-E-5 (15 g, 31 mmol) and sub10 (5.3 g, 31 mmol) were added to 300 ml of THF under a nitrogen atmosphere, and the mixture was stirred and refluxed. Then, potassium carbonate (12.9 g, 93 mmol) was dissolved in 39 ml of water, and then added thereto. Thereafter, it was stirred sufficiently, followed by adding bis(tri-tert-butylphosphine)palladium(0) (0.2 g, 0.3 mmol). After 12 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.1 g of Compound 1-37 (yield 79%, MS: [M+H]+=576).

### Preparation Example 1-38

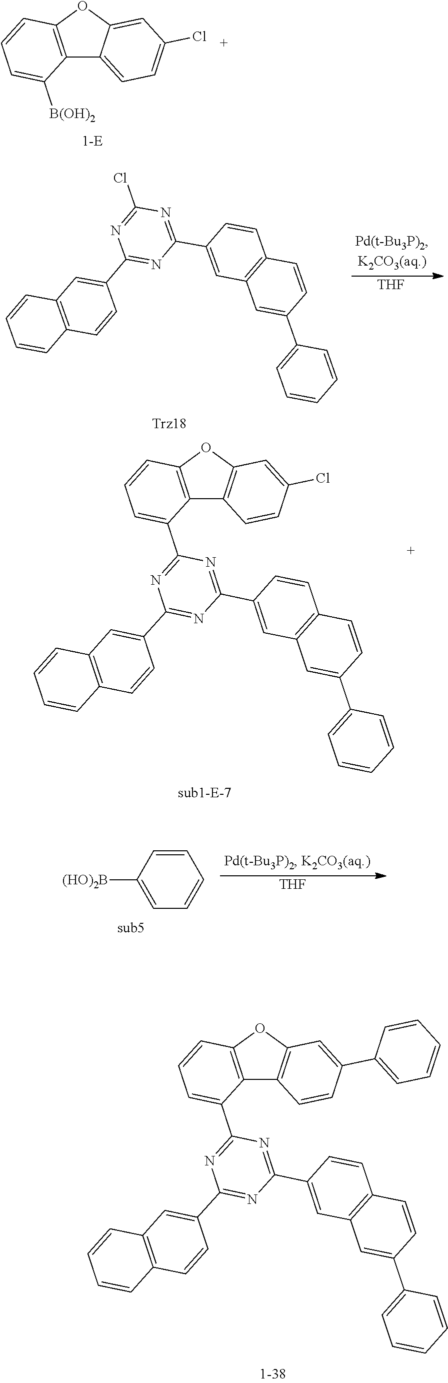

Compound 1-E (15 g, 60.9 mmol) and Trz18 (27 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 24.1 g of sub1-E-7 (yield 65%, MS: [M+H]+=610).

sub1-E-7 (15 g, 24.6 mmol) and sub5 (3 g, 24.6 mmol) were added to 300 ml of THF under a nitrogen atmosphere, and the mixture was stirred and refluxed. Then, potassium carbonate (10.2 g, 73.8 mmol) was dissolved in 31 ml of water, and then added thereto. Thereafter, it was stirred sufficiently, followed by adding bis(tri-tert-butylphosphine)palladium(0) (0.1 g, 0.2 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.1 g of Compound 1-38 (yield 63%, MS: [M+H]+=652).

### Preparation Example 1-39

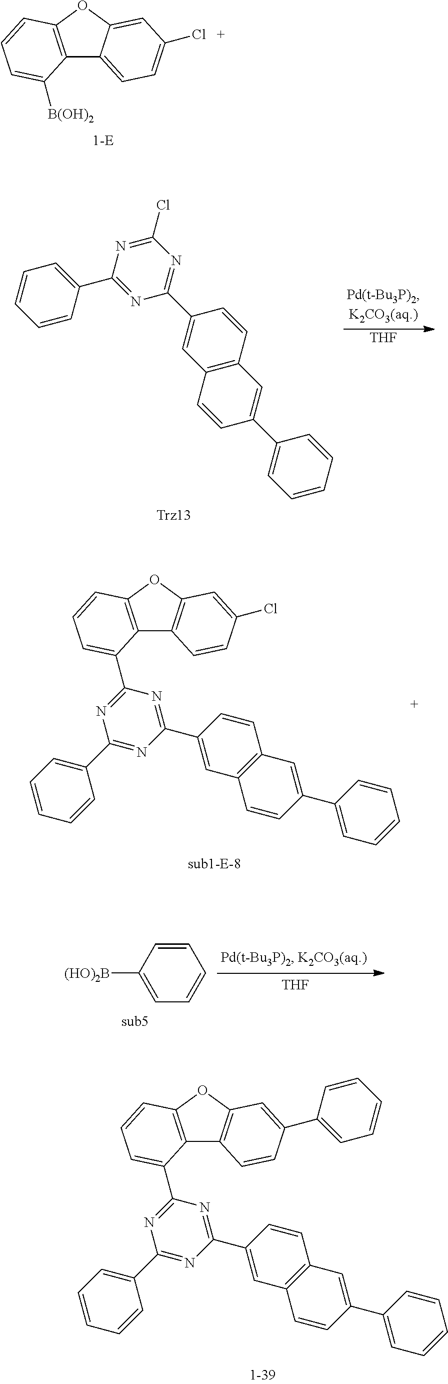

Compound 1-E (15 g, 60.9 mmol) and Trz13 (24 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 12 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 26.2 g of sub1-E-8 (yield 77%, MS: [M+H]+=560).

sub1-E-8 (15 g, 26.8 mmol) and sub5 (3.3 g, 26.8 mmol) were added to 300 ml of THF under a nitrogen atmosphere, and the mixture was stirred and refluxed. Then, potassium carbonate (11.1 g, 80.3 mmol) was dissolved in 33 ml of water, and then added thereto. Thereafter, it was stirred sufficiently, followed by adding bis(tri-tert-butylphosphine)palladium(0) (0.1 g, 0.3 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.9 g of Compound 1-39 (yield 68%, MS: [M+H]+=602).

### Preparation Example 1-40

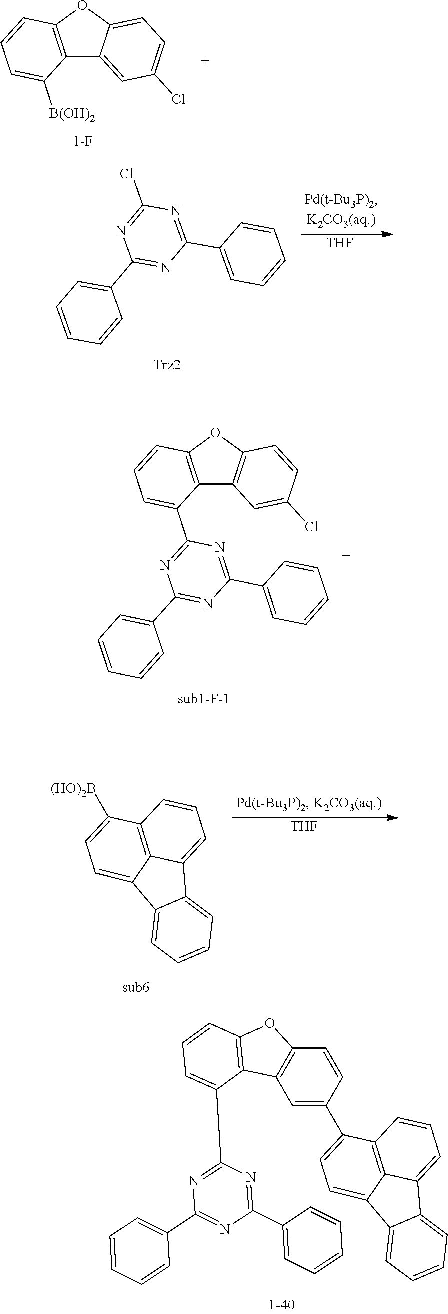

Compound 1-F (15 g, 60.9 mmol) and Trz2 (16.3 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 12 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 19.2 g of sub1-F-1 (yield 73%, MS: [M+H]+=434).

Compound 1-F (15 g, 34.6 mmol) and sub6 (8.5 g, 34.6 mmol) were added to 300 ml of THF under a nitrogen atmosphere, and the mixture was stirred and refluxed. Then, potassium carbonate (14.3 g, 103.7 mmol) was dissolved in 43 ml of water, and then added thereto. Thereafter, it was stirred sufficiently, followed by adding bis(tri-tert-butylphosphine)palladium(0) (0.2 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.7 g of Compound 1-40 (yield 71%, MS: [M+H]+=600).

### Preparation Example 1-41

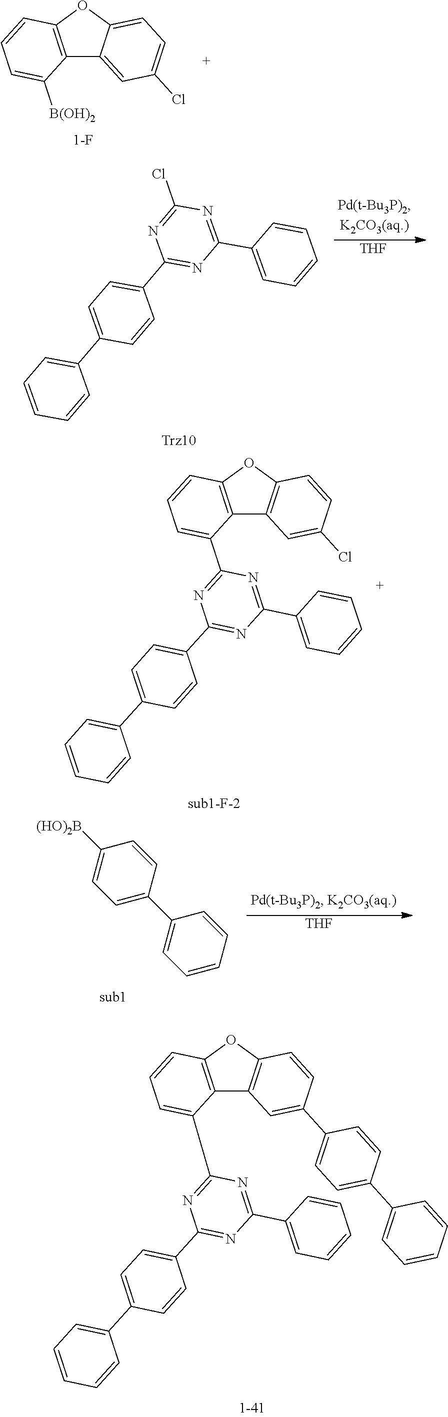

Compound 1-F (15 g, 60.9 mmol) and Trz10 (20.9 g, 60.9 mmol) were added to 300 ml of THF under a nitrogen atmosphere, and the mixture was stirred and refluxed. Then, potassium carbonate (25.2 g, 182.6 mmol) was dissolved in 76 ml of water, and then added thereto. Thereafter, it was stirred sufficiently, followed by adding bis(tri-tert-butylphosphine)palladium(0) (0.3 g, 0.6 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21.1 g of sub1-F-2 (yield 68%, MS: [M+H]+=510).

sub1-F-2 (15 g, 29.4 mmol) and sub1 (5.8 g, 29.4 mmol) were added to 300 ml of THF under a nitrogen atmosphere, and the mixture was stirred and refluxed. Then, potassium carbonate (12.2 g, 88.2 mmol) was dissolved in 37 ml of water, and then added thereto. Thereafter, it was stirred sufficiently, followed by adding bis(tri-tert-butylphosphine)palladium(0) (0.2 g, 0.3 mmol). After 12 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.2 g of Compound 1-41 (yield 77%, MS: [M+H]+=628).

### Preparation Example 1-42

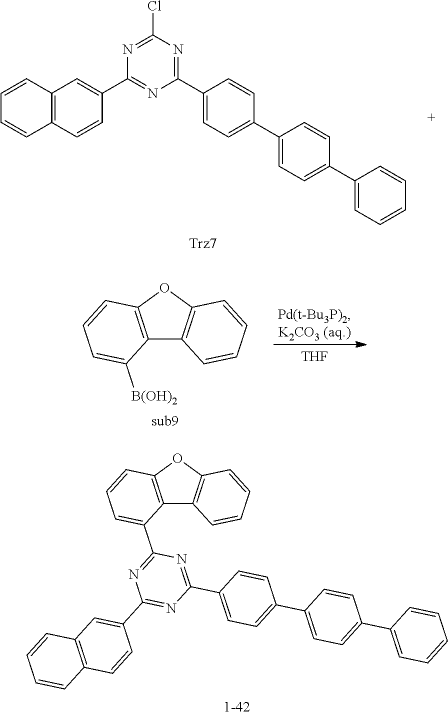

Trz7 (15 g, 31.9 mmol) and sub9 (6.8 g, 31.9 mmol) were added to 300 ml of THF under a nitrogen atmosphere, and the mixture was stirred and refluxed. Then, potassium carbonate (13.2 g, 95.8 mmol) was dissolved in 40 ml of water, and then added thereto. Thereafter, it was stirred sufficiently, followed by adding bis(tri-tert-butylphosphine)palladium(0) (0.2 g, 0.3 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.2 g of Compound 1-42 (yield 79%, MS: [M+H]+=602).

### Preparation Example 1-43

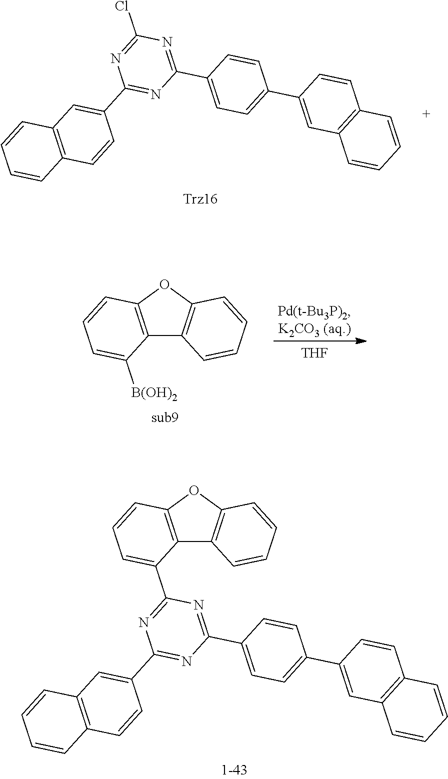 [SMILES: Clc1nc(-c2ccc(-c3ccc4ccccc4c3)cc2)nc(-c2ccc3ccccc3c2)n1 ; "THF" ; OB(O)c1cccc2oc3ccccc3c12 ; c1ccc2cc(-c3ccc(-c4nc(-c5ccc6ccccc6c5)nc(-c5cccc6oc7ccccc7c56)n4)cc3)ccc2c1]

Trz16 (15 g, 33.8 mmol) and sub9 (7.2 g, 33.8 mmol) were added to 300 ml of THF under a nitrogen atmosphere, and the mixture was stirred and refluxed. Then, potassium carbonate (14 g, 101.4 mmol) was dissolved in 42 ml of water, and then added thereto. Thereafter, it was stirred sufficiently, followed by adding bis(tri-tert-butylphosphine)palladium(0) (0.2 g, 0.3 mmol). After 12 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15 g of Compound 1-43 (yield 77%, MS: [M+H]+=576).

### Preparation Example 1-44

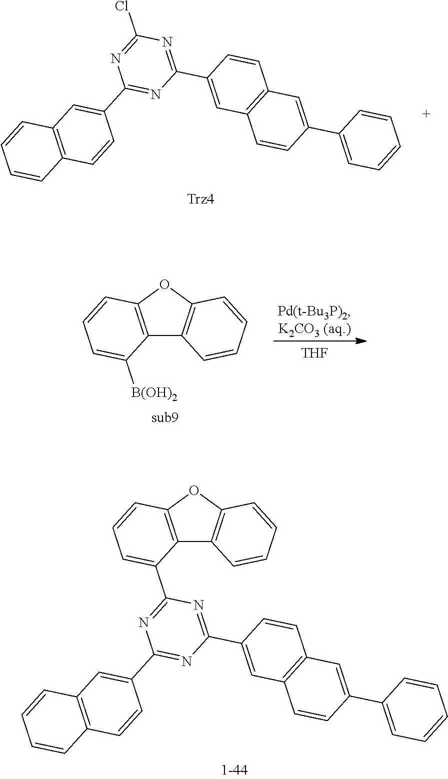 [SMILES: Clc1nc(-c2ccc3ccccc3c2)nc(-c2ccc3cc(-c4ccccc4)ccc3c2)n1 ; "THF" ; OB(O)c1cccc2oc3ccccc3c12 ; c1ccc(-c2ccc3cc(-c4nc(-c5ccc6ccccc6c5)nc(-c5cccc6oc7ccccc7c56)n4)ccc3c2)cc1]

Trz4 (15 g, 33.8 mmol) and sub9 (7.2 g, 33.8 mmol) were added to 300 ml of THF under a nitrogen atmosphere, and the mixture was stirred and refluxed. Then, potassium carbonate (14 g, 101.4 mmol) was dissolved in 42 ml of water, and then added thereto. Thereafter, it was stirred sufficiently, followed by adding bis(tri-tert-butylphosphine)palladium(0) (0.2 g, 0.3 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.2 g of Compound 1-44 (yield 73%, MS: [M+H]+=576).

### Preparation Example 1-45

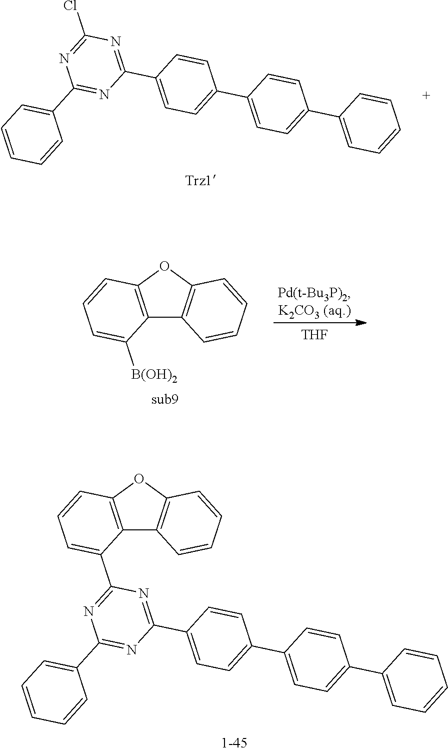 [SMILES: Clc1nc(-c2ccccc2)nc(-c2ccc(-c3ccc(-c4ccccc4)cc3)cc2)n1 ; "THF" ; OB(O)c1cccc2oc3ccccc3c12 ; c1ccc(-c2ccc(-c3ccc(-c4nc(-c5ccccc5)nc(-c5cccc6oc7ccccc7c56)n4)cc3)cc2)cc1]

Trz1′ (15 g, 35.7 mmol) and sub9 (7.6 g, 35.7 mmol) were added to 300 ml of THF under a nitrogen atmosphere, and the mixture was stirred and refluxed. Then, potassium carbonate (14.8 g, 107.2 mmol) was dissolved in 44 ml of water, and then added thereto. Thereafter, it was stirred sufficiently, followed by adding bis(tri-tert-butylphosphine)palladium(0) (0.2 g, 0.4 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.2 g of Compound 1-45 (yield 62%, MS: [M+H]+=552).

### Preparation Example 1-46

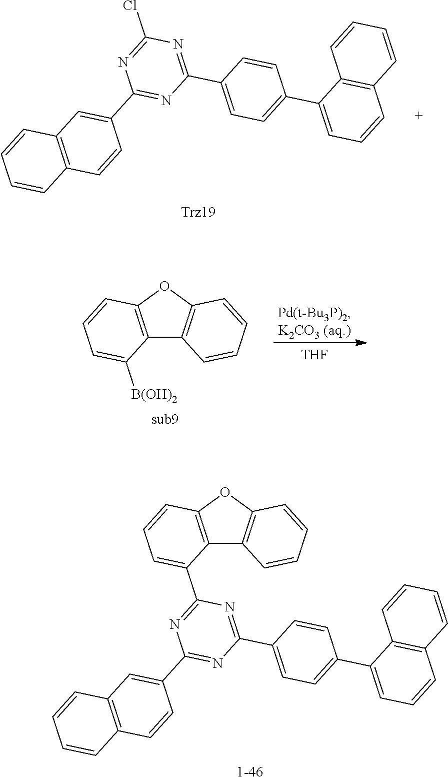 [SMILES: Clc1nc(-c2ccc(-c3cccc4ccccc34)cc2)nc(-c2ccc3ccccc3c2)n1 ; "THF" ; OB(O)c1cccc2oc3ccccc3c12 ; c1ccc2cc(-c3nc(-c4ccc(-c5cccc6ccccc56)cc4)nc(-c4cccc5oc6ccccc6c45)n3)ccc2c1]

Trz19 (15 g, 33.8 mmol) and sub9 (7.2 g, 33.8 mmol) were added to 300 ml of THF under a nitrogen atmosphere, and the mixture was stirred and refluxed. Then, potassium carbonate (14 g, 101.4 mmol) was dissolved in 42 ml of water, and then added thereto. Thereafter, it was stirred sufficiently, followed by adding bis(tri-tert-butylphosphine)palladium(0) (0.2 g, 0.3 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.6 g of Compound 1-46 (yield 70%, MS: [M+H]+=576).

### Preparation Example 1-47

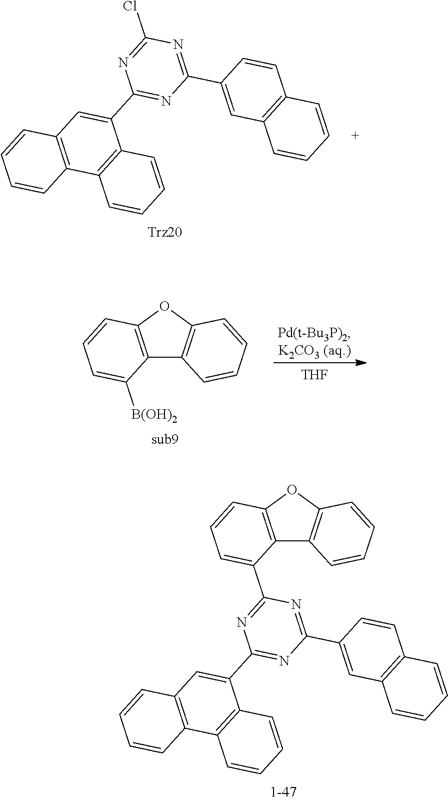 [SMILES: Clc1nc(-c2ccc3ccccc3c2)nc(-c2cc3ccccc3c3ccccc23)n1 ; "THF" ; OB(O)c1cccc2oc3ccccc3c12 ; c1ccc2cc(-c3nc(-c4cc5ccccc5c5ccccc45)nc(-c4cccc5oc6ccccc6c45)n3)ccc2c1]

Trz20 (15 g, 35.9 mmol) and sub9 (7.6 g, 35.9 mmol) were added to 300 ml of THF under a nitrogen atmosphere, and the mixture was stirred and refluxed. Then, potassium carbonate (14.9 g, 107.7 mmol) was dissolved in 45 ml of water, and then added thereto. Thereafter, it was stirred sufficiently, followed by adding bis(tri-tert-butylphosphine)palladium(0) (0.2 g, 0.4 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15 g of Compound 1-47 (yield 76%, MS: [M+H]+=550).

### Preparation Example 1-48

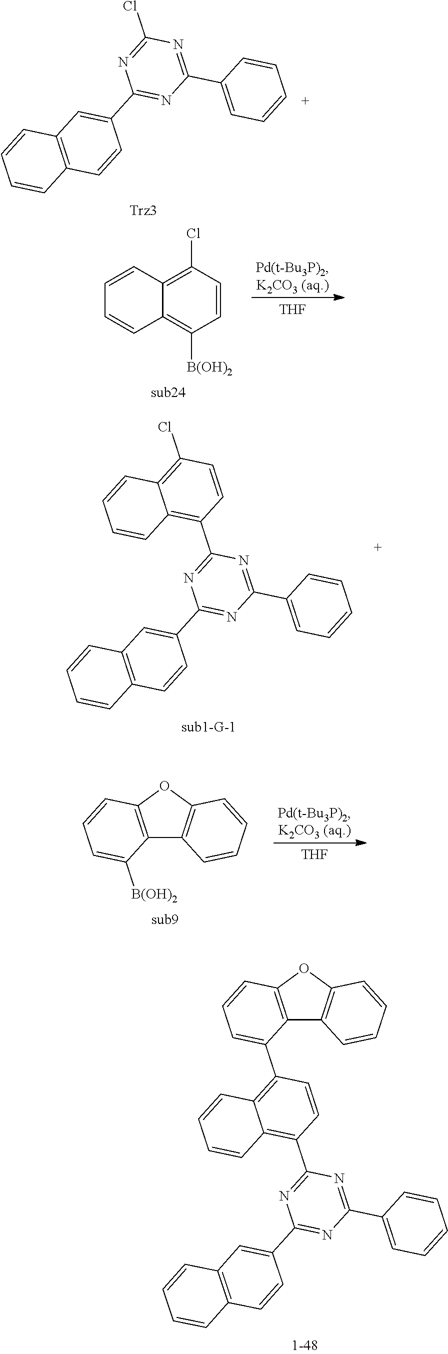

Trz3 (15 g, 47.2 mmol) and sub24 (9.7 g, 47.2 mmol) were added to 300 ml of THF under a nitrogen atmosphere, and the mixture was stirred and refluxed. Then, potassium carbonate (19.6 g, 141.6 mmol) was dissolved in 59 ml of water, and then added thereto. Thereafter, it was stirred sufficiently, followed by adding bis(tri-tert-butylphosphine)palladium(0) (0.2 g, 0.5 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13 g of sub1-G-1 (yield 62%, MS: [M+H]+=444).

sub1-G-1 (15 g, 33.8 mmol) and sub9 (7.2 g, 33.8 mmol) were added to 300 ml of THF under a nitrogen atmosphere, and the mixture was stirred and refluxed. Then, potassium carbonate (14 g, 101.4 mmol) was dissolved in 42 ml of water, and then added thereto. Thereafter, it was stirred sufficiently, followed by adding bis(tri-tert-butylphosphine)palladium(0) (0.2 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.2 g of Compound 1-48 (yield 78%, MS: [M+H]+=576).

### Preparation Example 1-49

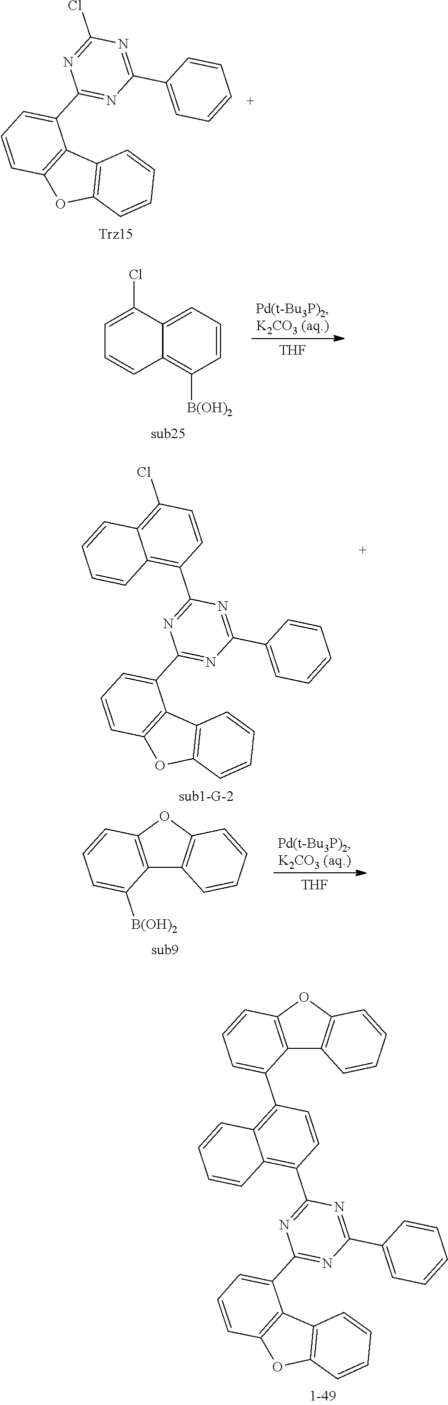

Trz15 (15 g, 41.9 mmol) and sub25 (8.7 g, 41.9 mmol) were added to 300 ml of THF under a nitrogen atmosphere, and the mixture was stirred and refluxed. Then, potassium carbonate (17.4 g, 125.8 mmol) was dissolved in 52 ml of water, and then added thereto. Thereafter, it was stirred sufficiently, followed by adding bis(tri-tert-butylphosphine)palladium(0) (0.2 g, 0.4 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.6 g of sub1-G-2 (yield 62%, MS: [M+H]+=484).

sub1-G-2 (15 g, 31 mmol) and sub9 (6.6 g, 31 mmol) were added to 300 ml of THF under a nitrogen atmosphere, and the mixture was stirred and refluxed. Then, potassium carbonate (12.9 g, 93 mmol) was dissolved in 39 ml of water, and then added thereto. Thereafter, it was stirred sufficiently, followed by adding bis(tri-tert-butylphosphine)palladium(0) (0.2 g, 0.3 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.7 g of Compound 1-49 (yield 72%, MS: [M+H]+=616).

### Preparation Example 1-50

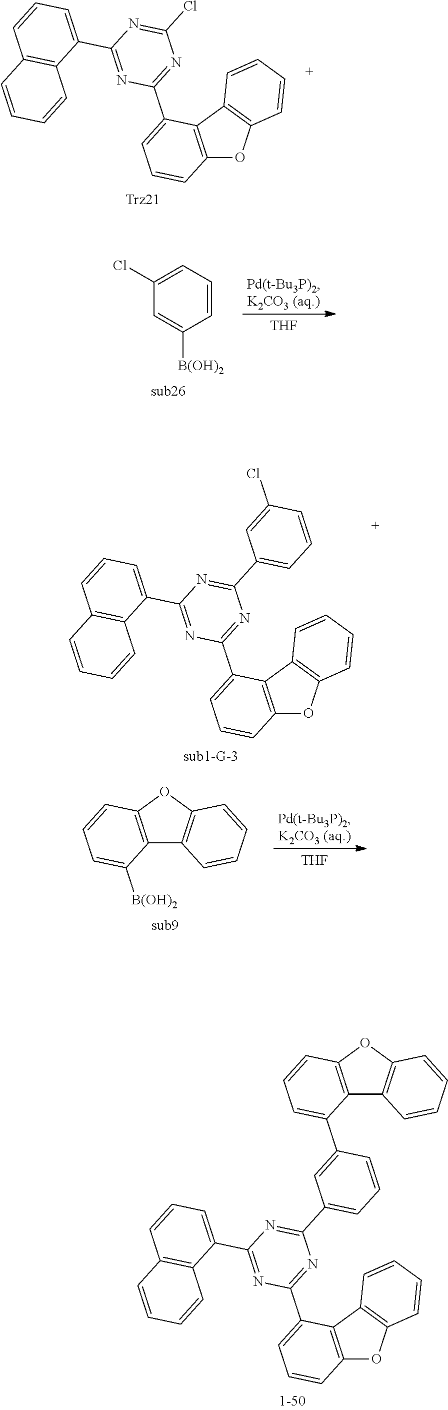

Trz21 (15 g, 36.8 mmol) and sub26 (5.8 g, 36.8 mmol) were added to 300 ml of THF under a nitrogen atmosphere, and the mixture was stirred and refluxed. Then, potassium carbonate (15.2 g, 110.3 mmol) was dissolved in 46 ml of water, and then added thereto. Thereafter, it was stirred sufficiently, followed by adding bis(tri-tert-butylphosphine)palladium(0) (0.2 g, 0.4 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.8 g of sub1-G-3 (yield 72%, MS: [M+H]+=484).

sub1-G-3 (15 g, 31 mmol) and sub9 (6.6 g, 31 mmol) were added to 300 ml of THF under a nitrogen atmosphere, and the mixture was stirred and refluxed. Then, potassium carbonate (12.9 g, 93 mmol) was dissolved in 39 ml of water, and then added thereto. Thereafter, it was stirred sufficiently, followed by adding bis(tri-tert-butylphosphine)palladium(0) (0.2 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.2 g of Compound 1-50 (yield 69%, MS: [M+H]+=616).

### Preparation Example 1-51

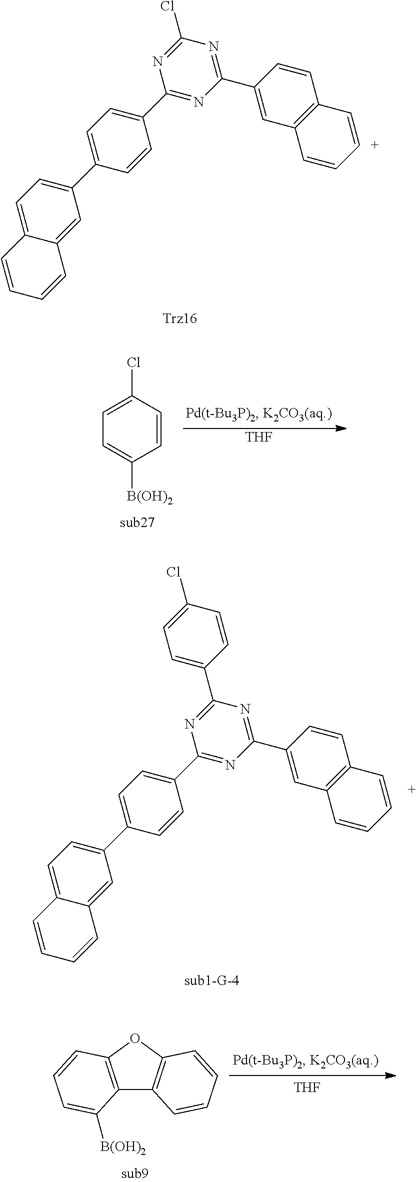 [SMILES: Clc1nc(-c2ccc(-c3ccc4ccccc4c3)cc2)nc(-c2ccc3c(c2)C=C[C+]=C3)n1 ; OB(O)c1ccc(Cl)cc1 ; Clc1ccc(-c2nc(-c3ccc(-c4ccc5ccccc5c4)cc3)nc(-c3ccc4ccccc4c3)n2)cc1 ; "THF" ; "THF" ; OB(O)c1cccc2oc3ccccc3c12]

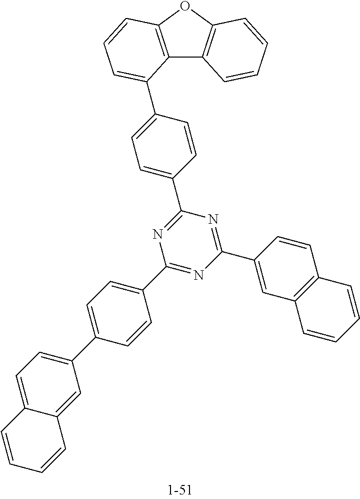 [SMILES: c1ccc2cc(-c3ccc(-c4nc(-c5ccc(-c6cccc7oc8ccccc8c67)cc5)nc(-c5ccc6ccccc6c5)n4)cc3)ccc2c1]

Trz16 (15 g, 33.8 mmol) and sub27 (5.3 g, 33.8 mmol) were added to 300 ml of THF under a nitrogen atmosphere, and the mixture was stirred and refluxed. Then, potassium carbonate (14 g, 101.4 mmol) was dissolved in 42 ml of water, and then added thereto. Thereafter, it was stirred sufficiently, followed by adding bis(tri-tert-butylphosphine)palladium(0) (0.2 g, 0.3 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.3 g of sub1-G-4 (yield 76%, MS: [M+H]+=520).

sub1-G-4 (15 g, 28.8 mmol) and sub9 (6.1 g, 28.8 mmol) were added to 300 ml of THF under a nitrogen atmosphere, and the mixture was stirred and refluxed. Then, potassium carbonate (12 g, 86.5 mmol) was dissolved in 36 ml of water, and then added thereto. Thereafter, it was stirred sufficiently, followed by adding bis(tri-tert-butylphosphine)palladium(0) (0.1 g, 0.3 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.3 g of Compound 1-51 (yield 71%, MS: [M+H]+=652).

### Preparation Example 1-52

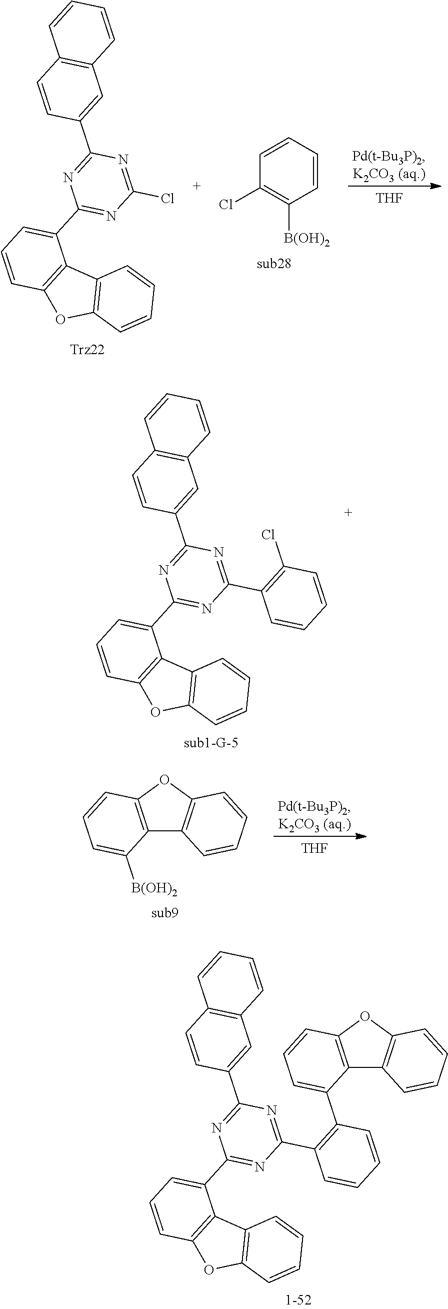

Trz22 (15 g, 36.8 mmol) and sub28 (5.8 g, 36.8 mmol) were added to 300 ml of THF under a nitrogen atmosphere, and the mixture was stirred and refluxed. Then, potassium carbonate (15.2 g, 110.3 mmol) was dissolved in 46 ml of water, and then added thereto. Thereafter, it was stirred sufficiently, followed by adding bis(tri-tert-butylphosphine)palladium(0) (0.2 g, 0.4 mmol). After 12 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.8 g of sub1-G-5 (yield 72%, MS: [M+H]+=484).

sub1-G-5 (15 g, 31 mmol) and sub9 (6.6 g, 31 mmol) were added to 300 ml of THF under a nitrogen atmosphere, and the mixture was stirred and refluxed. Then, potassium carbonate (12.9 g, 93 mmol) was dissolved in 39 ml of water, and then added thereto. Thereafter, it was stirred sufficiently, followed by adding bis(tri-tert-butylphosphine)palladium(0) (0.2 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13 g of Compound 1-52 (yield 68%, MS: [M+H]+=616).

### Preparation Example 1-53

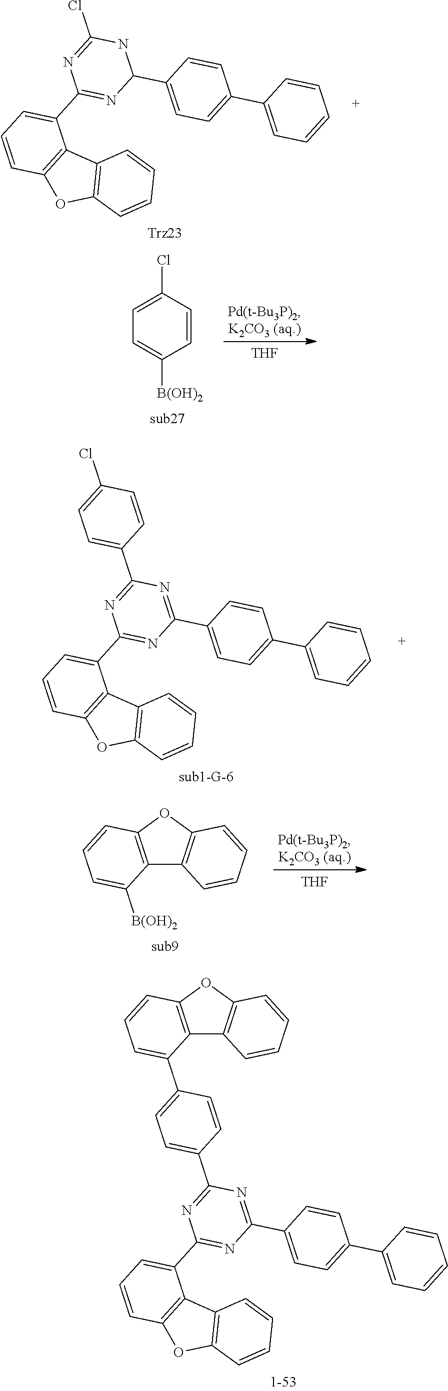

Trz23 (15 g, 34.6 mmol) and sub27 (5.4 g, 34.6 mmol) were added to 300 ml of THF under a nitrogen atmosphere, and the mixture was stirred and refluxed. Then, potassium carbonate (14.3 g, 103.7 mmol) was dissolved in 43 ml of water, and then added thereto. Thereafter, it was stirred sufficiently, followed by adding bis(tri-tert-butylphosphine)palladium(0) (0.2 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.3 g of sub1-G-6 (yield 64%, MS: [M+H]+=510).

sub1-G-6 (15 g, 31 mmol) and sub9 (6.6 g, 31 mmol) were added to 300 ml of THF under a nitrogen atmosphere, and the mixture was stirred and refluxed. Then, potassium carbonate (12.9 g, 93 mmol) was dissolved in 39 ml of water, and then added thereto. Thereafter, it was stirred sufficiently, followed by adding bis(tri-tert-butylphosphine)palladium(0) (0.2 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13 g of Compound 1-53 (yield 68%, MS: [M+H]+=616).

### Preparation Example 1-54

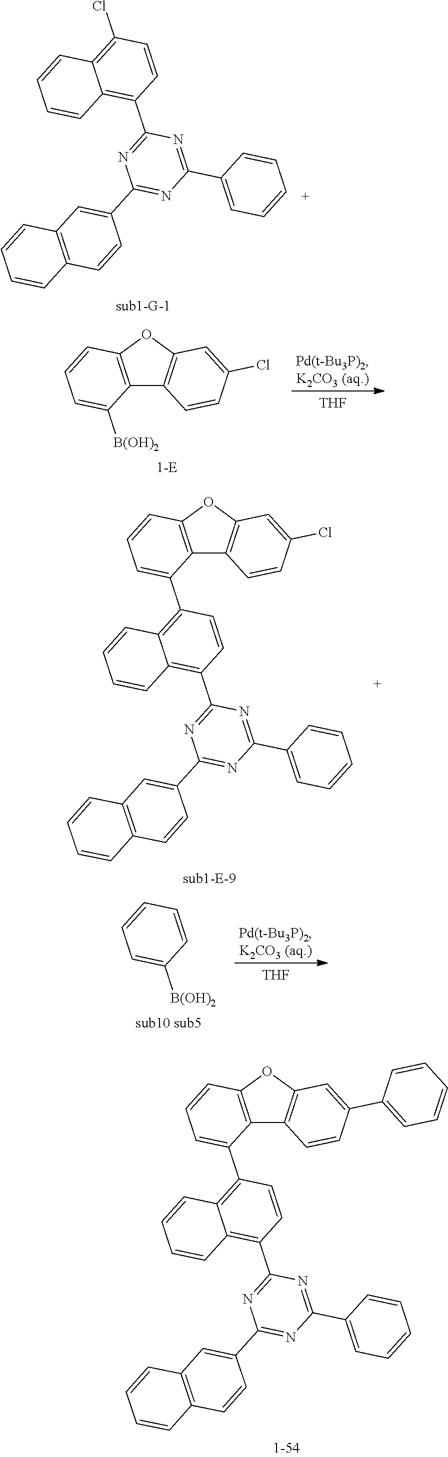

sub1-G-1 (15 g, 33.8 mmol) and Compound 1-E (8.3 g, 33.8 mmol) were added to 300 ml of THF under a nitrogen atmosphere, and the mixture was stirred and refluxed. Then, potassium carbonate (14 g, 101.4 mmol) was dissolved in 42 ml of water, and then added thereto. Thereafter, it was stirred sufficiently, followed by adding bis(tri-tert-butylphosphine)palladium(0) (0.2 g, 0.3 mmol). After 8 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.4 g of sub1-E-9 (yield 70%, MS: [M+H]+=610).

sub1-E-9 (15 g, 24.6 mmol) and sub5 (3 g, 24.6 mmol) were added to 300 ml of THF under a nitrogen atmosphere, and the mixture was stirred and refluxed. Then, potassium carbonate (10.2 g, 73.8 mmol) was dissolved in 31 ml of water, and then added thereto. Thereafter, it was stirred sufficiently, followed by adding bis(tri-tert-butylphosphine)palladium(0) (0.1 g, 0.2 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.2 g of Compound 1-54 (yield 76%, MS: [M+H]+=652).

### Preparation Example 1-55

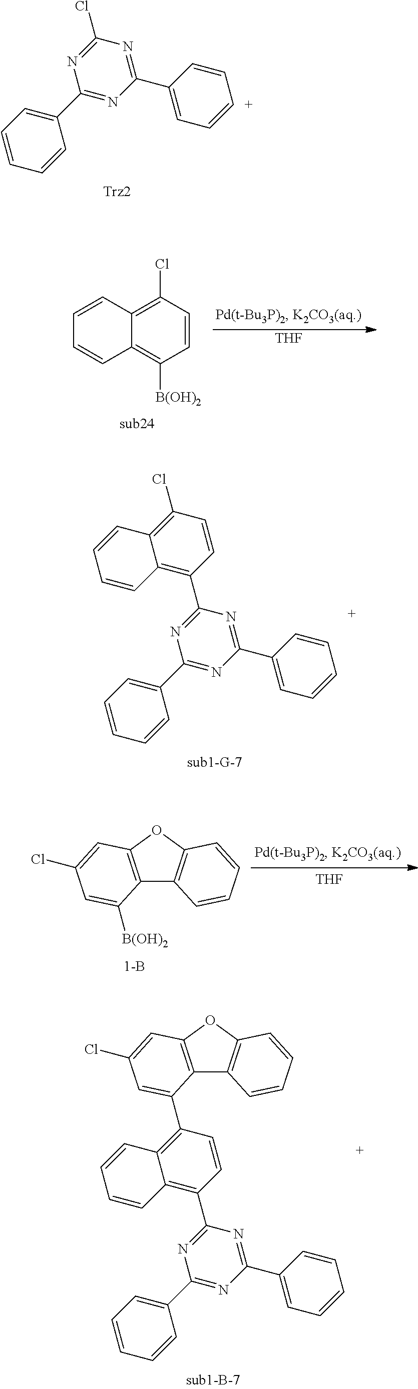

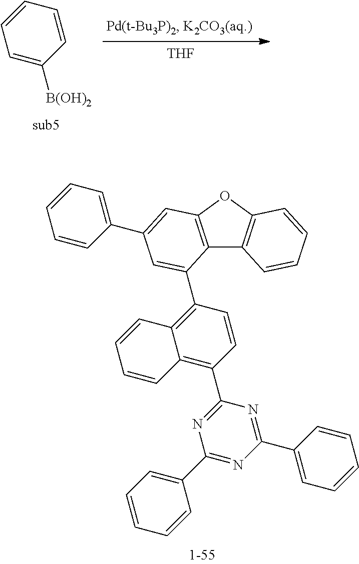 [SMILES: "THF" ; OB(O)c1ccccc1 ; c1ccc(-c2cc(-c3ccc(-c4nc(-c5ccccc5)nc(-c5ccccc5)n4)c4ccccc34)c3c(c2)oc2ccccc23)cc1]

Trz2 (15 g, 56 mmol) and sub24 (11.6 g, 56 mmol) were added to 300 ml of THF under a nitrogen atmosphere, and the mixture was stirred and refluxed. Then, potassium carbonate (23.2 g, 168.1 mmol) was dissolved in 70 ml of water, and then added thereto. Thereafter, it was stirred sufficiently, followed by adding bis(tri-tert-butylphosphine)palladium(0) (0.3 g, 0.6 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.6 g of sub1-G-7 (yield 71%, MS: [M+H]+=394).

sub1-G-7 (15 g, 38.1 mmol) and Compound 1-B (9.4 g, 38.1 mmol) were added to 300 ml of THF under a nitrogen atmosphere, and the mixture was stirred and refluxed. Then, potassium carbonate (15.8 g, 114.3 mmol) was dissolved in 47 ml of water, and then added thereto. Thereafter, it was stirred sufficiently, followed by adding bis(tri-tert-butylphosphine)palladium(0) (0.2 g, 0.4 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.8 g of sub1-B-7 (yield 65%, MS: [M+H]+=560).

sub1-B-7 (15 g, 26.8 mmol) and sub5 (3.3 g, 26.8 mmol) were added to 300 ml of THF under a nitrogen atmosphere, and the mixture was stirred and refluxed. Then, potassium carbonate (11.1 g, 80.3 mmol) was dissolved in 33 ml of water, and then added thereto. Thereafter, it was stirred sufficiently, followed by adding bis(tri-tert-butylphosphine)palladium(0) (0.1 g, 0.3 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.9 g of Compound 1-55 (yield 80%, MS: [M+H]+=602).

### Preparation Example 1-56

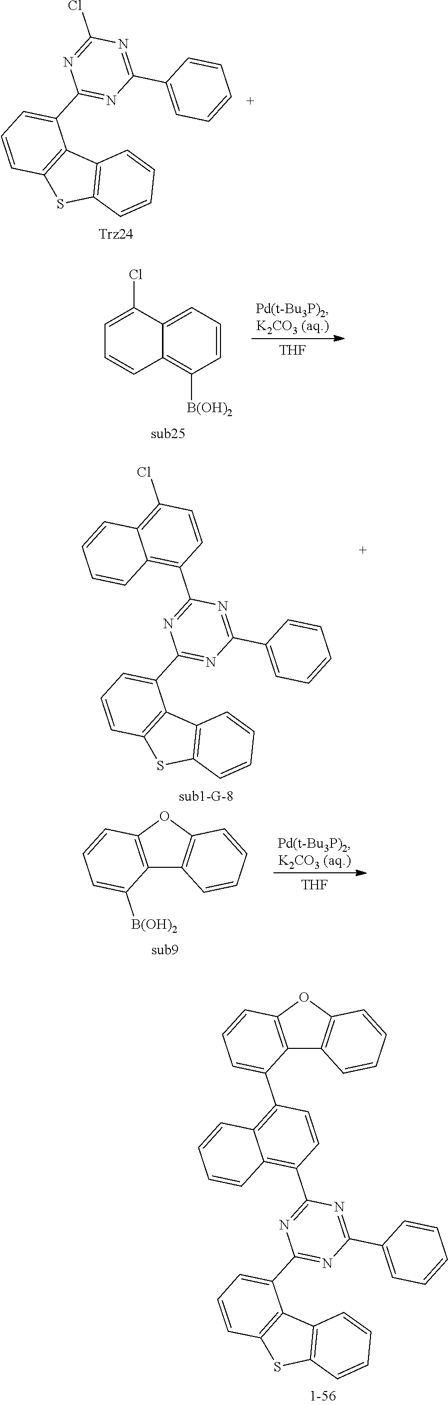

Trz24 (15 g, 38.1 mmol) and Compound sub25 (9.4 g, 38.1 mmol) were added to 300 ml of THF under a nitrogen atmosphere, and the mixture was stirred and refluxed. Then, potassium carbonate (15.8 g, 114.3 mmol) was dissolved in 47 ml of water, and then added thereto. Thereafter, it was stirred sufficiently, followed by adding bis(tri-tert-butylphosphine)palladium(0) (0.2 g, 0.4 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.8 g of Compound sub1-G-8 (yield 65%, MS: [M+H]+=560).

Compound sub1-G-8 (15 g, 30 mmol) and Compound sub9 (6.4 g, 30 mmol) were added to 300 ml of THF under a nitrogen atmosphere, and the mixture was stirred and refluxed. Then, potassium carbonate (12.4 g, 90 mmol) was dissolved in 37 ml of water, and then added thereto. Thereafter, it was stirred sufficiently, followed by adding bis(tri-tert-butylphosphine)palladium(0) (0.2 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.4 g of Compound 1-56 (yield 71%, MS: [M+H]+=632).

### Preparation Example 1-57

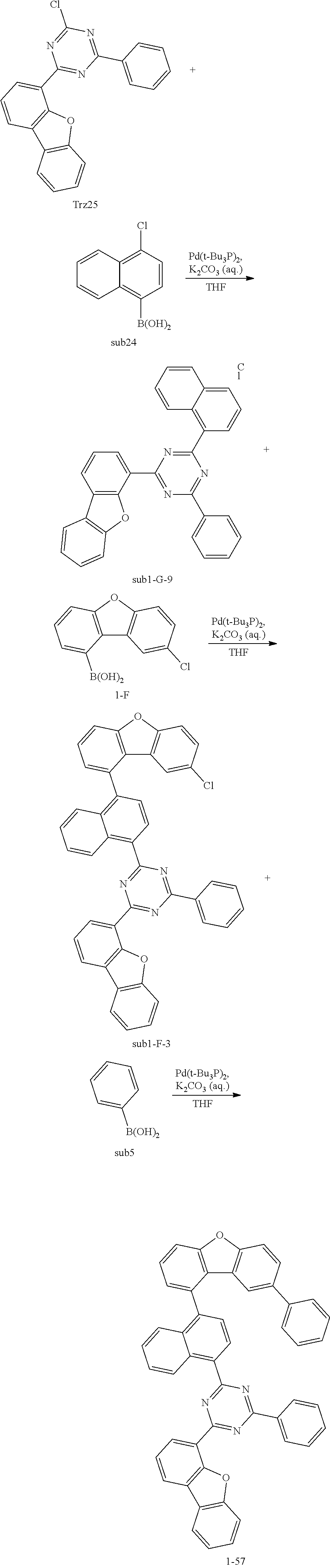

Trz25 (15 g, 41.9 mmol) and sub24 (8.7 g, 41.9 mmol) were added to 300 ml of THF under a nitrogen atmosphere, and the mixture was stirred and refluxed. Then, potassium carbonate (17.4 g, 125.8 mmol) was dissolved in 52 ml of water, and then added thereto. Thereafter, it was stirred sufficiently, followed by adding bis(tri-tert-butylphosphine)palladium(0) (0.2 g, 0.4 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.4 g of sub1-G-9 (yield 61%, MS: [M+H]+=484).

sub1-G-9 (15 g, 31 mmol) and Compound 1-F (7.6 g, 31 mmol) were added to 300 ml of THF under a nitrogen atmosphere, and the mixture was stirred and refluxed. Then, potassium carbonate (12.9 g, 93 mmol) was dissolved in 39 ml of water, and then added thereto. Thereafter, it was stirred sufficiently, followed by adding bis(tri-tert-butylphosphine)palladium(0) (0.2 g, 0.3 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.5 g of sub1-F-3 (yield 62%, MS: [M+H]+=650).

sub1-F-3 (15 g, 23.1 mmol) and sub5 (2.8 g, 23.1 mmol) were added to 300 ml of THF under a nitrogen atmosphere, and the mixture was stirred and refluxed. Then, potassium carbonate (9.6 g, 69.2 mmol) was dissolved in 29 ml of water, and then added thereto. Thereafter, it was stirred sufficiently, followed by adding bis(tri-tert-butylphosphine)palladium(0) (0.1 g, 0.2 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.8 g of Compound 1-57 (yield 80%, MS: [M+H]+=692).

### Preparation Example 1-58

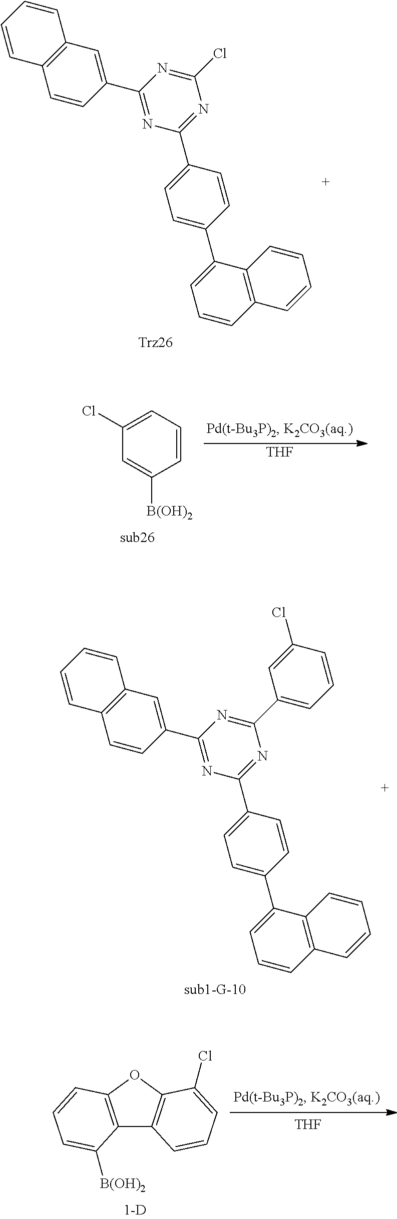 [SMILES: Clc1nc(-c2ccc(-c3cccc4ccccc34)cc2)nc(-c2ccc3ccccc3c2)n1 ; OB(O)c1cccc(Cl)c1 ; Clc1cccc(-c2nc(-c3ccc(-c4cccc5ccccc45)cc3)nc(-c3ccc4ccccc4c3)n2)c1 ; "THF" ; "THF" ; OB(O)c1cccc2oc3c(Cl)cccc3c12]

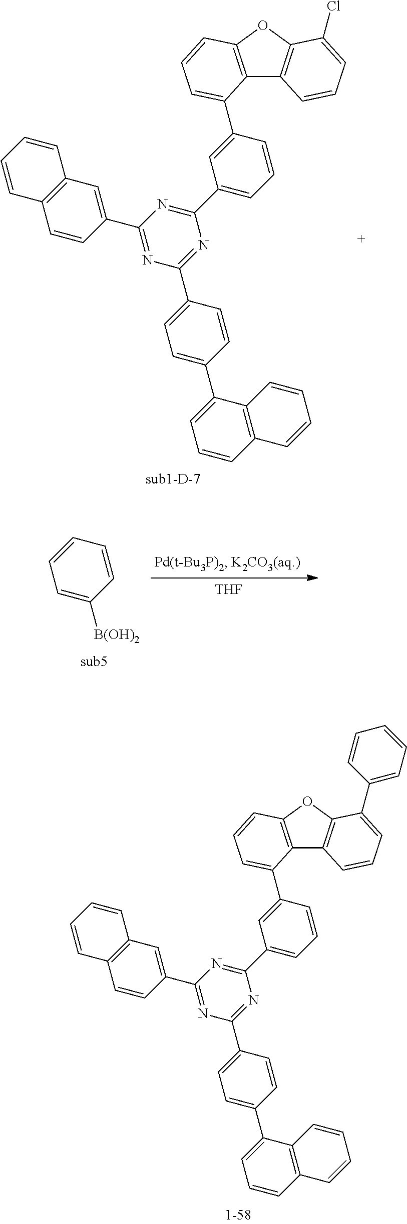 [SMILES: Clc1cccc2c1oc1cccc(-c3cccc(-c4nc(-c5ccc(-c6cccc7ccccc67)cc5)nc(-c5ccc6ccccc6c5)n4)c3)c12 ; "THF" ; OB(O)c1ccccc1 ; c1ccc(-c2cccc3c2oc2cccc(-c4cccc(-c5nc(-c6ccc(-c7cccc8ccccc78)cc6)nc(-c6ccc7ccccc7c6)n5)c4)c23)cc1]

Trz26 (15 g, 33.8 mmol) and sub26 (5.3 g, 33.8 mmol) were added to 300 ml of THF under a nitrogen atmosphere, and the mixture was stirred and refluxed. Then, potassium carbonate (14 g, 101.4 mmol) was dissolved in 42 ml of water, and then added thereto. Thereafter, it was stirred sufficiently, followed by adding bis(tri-tert-butylphosphine)palladium(0) (0.2 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.5 g of sub1-G-10 (yield 60%, MS: [M+H]+=520).

sub1-G-10 (15 g, 28.8 mmol) and Compound 1-D (7.1 g, 28.8 mmol) were added to 300 ml of THF under a nitrogen atmosphere, and the mixture was stirred and refluxed. Then, potassium carbonate (12 g, 86.5 mmol) was dissolved in 36 ml of water, and then added thereto. Thereafter, it was stirred sufficiently, followed by adding bis(tri-tert-butylphosphine)palladium(0) (0.1 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15 g of sub1-D-7 (yield 76%, MS: [M+H]+=686).

sub1-D-7 (15 g, 21.9 mmol) and sub5 (2.7 g, 21.9 mmol) were added to 300 ml of THF under a nitrogen atmosphere, and the mixture was stirred and refluxed. Then, potassium carbonate (9.1 g, 65.6 mmol) was dissolved in 27 ml of water, and then added thereto. Thereafter, it was stirred sufficiently, followed by adding bis(tri-tert-butylphosphine)palladium(0) (0.1 g, 0.2 mmol). After 12 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 9.9 g of Compound 1-58 (yield 62%, MS: [M+H]+=728).

### Preparation Example 1-59

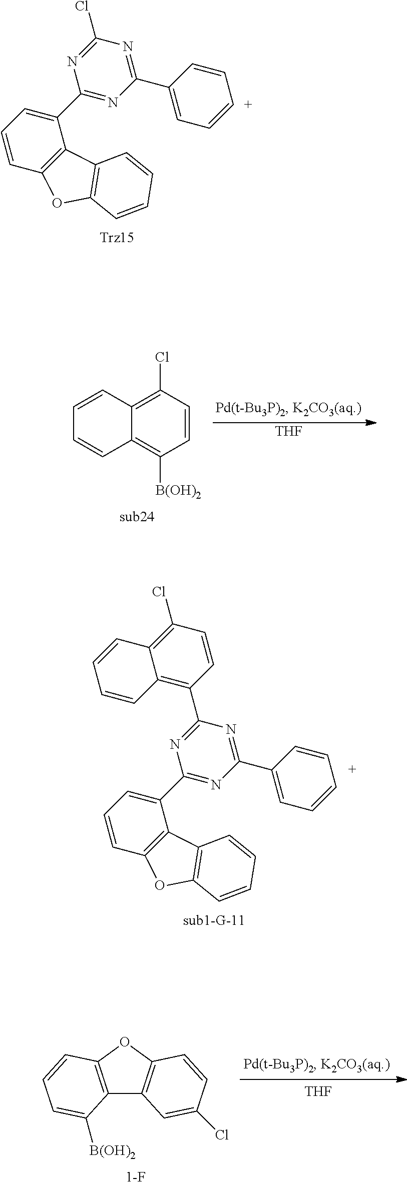 [SMILES: Clc1nc(C2=CC=[C+]C=C2)nc(-c2cccc3oc4ccccc4c23)n1 ; OB(O)c1ccc(Cl)c2ccccc12 ; Clc1ccc(-c2nc(-c3ccccc3)nc(-c3cccc4oc5ccccc5c34)n2)c2ccccc12 ; "THF" ; "THF" ; OB(O)c1cccc2oc3ccc(Cl)cc3c12]

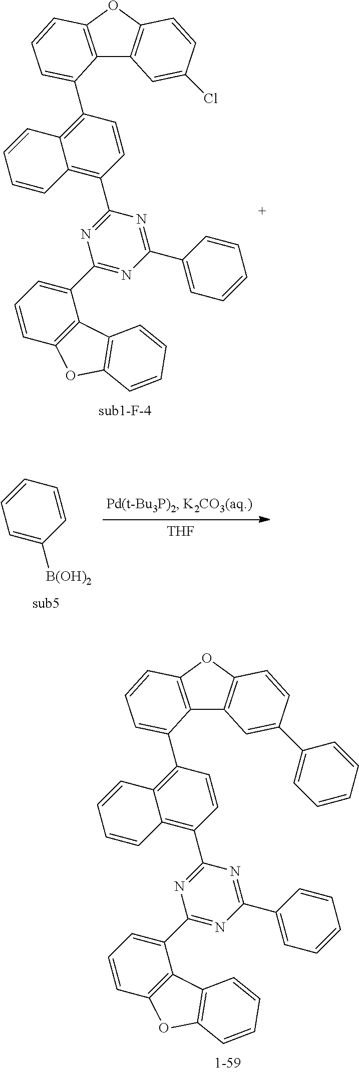 [SMILES: Clc1ccc2oc3cccc(-c4ccc(-c5nc(-c6ccccc6)nc(-c6cccc7oc8ccccc8c67)n5)c5ccccc45)c3c2c1 ; "THF" ; OB(O)c1ccccc1 ; c1ccc(-c2ccc3oc4cccc(-c5ccc(-c6nc(-c7ccccc7)nc(-c7cccc8oc9ccccc9c78)n6)c6ccccc56)c4c3c2)cc1]

Trz15 (15 g, 41.9 mmol) and Compound sub24 (8.7 g, 41.9 mmol) were added to 300 ml of THF under a nitrogen atmosphere, and the mixture was stirred and refluxed. Then, potassium carbonate (17.4 g, 125.8 mmol) was dissolved in 52 ml of water, and then added thereto. Thereafter, it was stirred sufficiently, followed by adding bis(tri-tert-butylphosphine)palladium(0) (0.2 g, 0.4 mmol). After 11 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.4 g of Compound sub1-G-11 (yield 61%, MS: [M+H]+=484).

sub1-G-11 (15 g, 28.8 mmol) and Compound 1-F (7.1 g, 28.8 mmol) were added to 300 ml of THF under a nitrogen atmosphere, and the mixture was stirred and refluxed. Then, potassium carbonate (12 g, 86.5 mmol) was dissolved in 36 ml of water, and then added thereto. Thereafter, it was stirred sufficiently, followed by adding bis(tri-tert-butylphosphine)palladium(0) (0.1 g, 0.3 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15 g of Compound sub1-F-4 (yield 76%, MS: [M+H]+=686).

sub1-F-4 (15 g, 23.1 mmol) and sub5 (2.8 g, 23.1 mmol) were added to 300 ml of THF under a nitrogen atmosphere, and the mixture was stirred and refluxed. Then, potassium carbonate (9.6 g, 69.2 mmol) was dissolved in 29 ml of water, and then added thereto. Thereafter, it was stirred sufficiently, followed by adding bis(tri-tert-butylphosphine)palladium(0) (0.1 g, 0.2 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.1 g of Compound 1-59 (yield 76%, MS: [M+H]+=692).

### Preparation Example 1-60

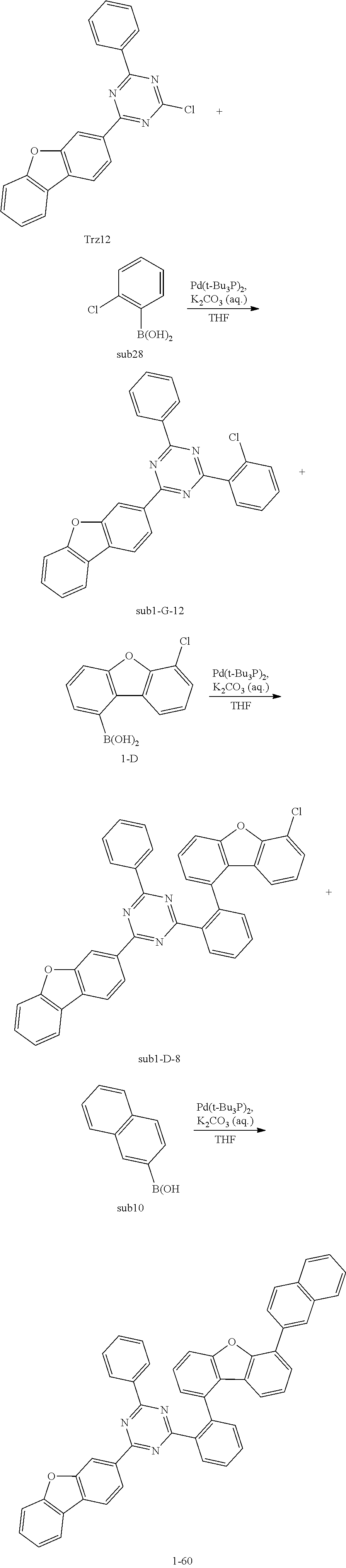

Trz12 (15 g, 41.9 mmol) and sub28 (6.6 g, 41.9 mmol) were added to 300 ml of THF under a nitrogen atmosphere, and the mixture was stirred and refluxed. Then, potassium carbonate (17.4 g, 125.8 mmol) was dissolved in 52 ml of water, and then added thereto. Thereafter, it was stirred sufficiently, followed by adding bis(tri-tert-butylphosphine)palladium(0) (0.2 g, 0.4 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.1 g of sub1-G-12 (yield 61%, MS: [M+H]+=434).

sub1-G-12 (15 g, 34.6 mmol) and Compound 1-D (8.5 g, 34.6 mmol) were added to 300 ml of THF under a nitrogen atmosphere, and the mixture was stirred and refluxed. Then, potassium carbonate (14.3 g, 103.7 mmol) was dissolved in 43 ml of water, and then added thereto. Thereafter, it was stirred sufficiently, followed by adding bis(tri-tert-butylphosphine)palladium(0) (0.2 g, 0.3 mmol). After 9 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.6 g of sub1-D-8 (yield 79%, MS: [M+H]+=500).

sub1-D-8 (15 g, 25 mmol) and sub10 (4.3 g, 25 mmol) were added to 300 ml of THF under a nitrogen atmosphere, and the mixture was stirred and refluxed. Then, potassium carbonate (10.4 g, 75 mmol) was dissolved in 31 ml of water, and then added thereto. Thereafter, it was stirred sufficiently, followed by adding bis(tri-tert-butylphosphine)palladium(0) (0.1 g, 0.2 mmol). After 10 hours of reaction, it was cooled to room temperature and the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.3 g of Compound 1-60 (yield 77%, MS: [M+H]+=692).

### Preparation Example 2-1

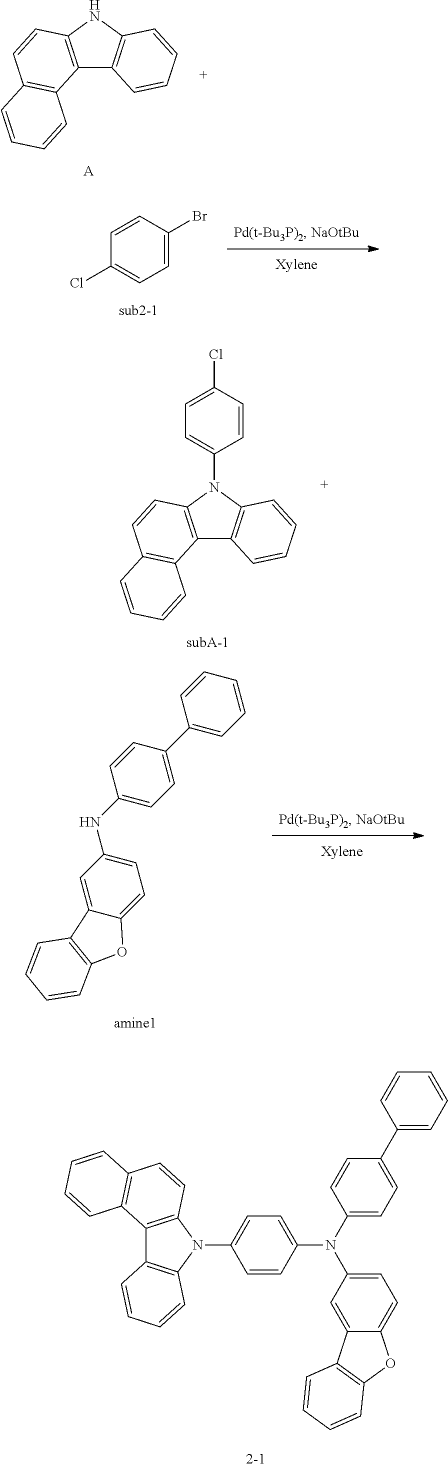

Compound A (10 g, 46 mmol), sub2-1 (9.3 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 8.7 g of subA-1 (yield 58%, MS: [M+H]+=328).

subA-1 (10 g, 30.5 mmol), amine1 (10.7 g, 32 mmol), and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 10.1 g of Compound 2-1 (yield 53%, MS: [M+H]+=627).

### Preparation Example 2-2

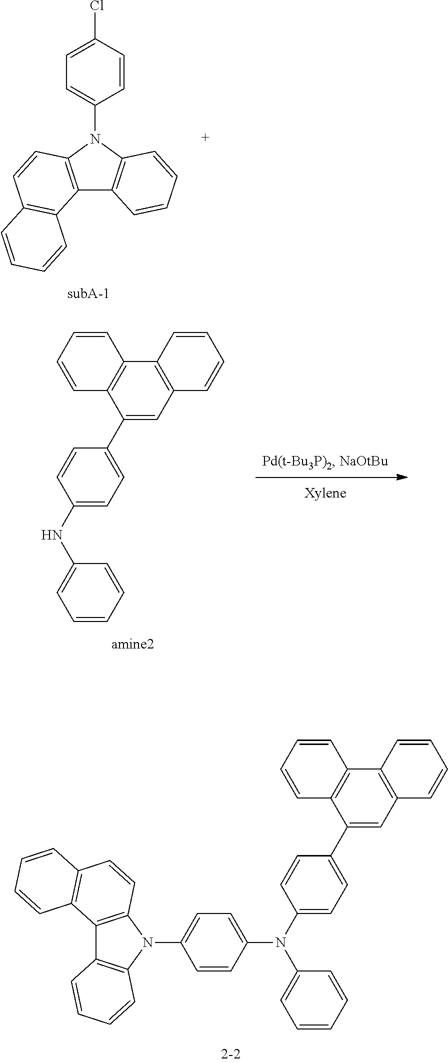 [SMILES: Clc1ccc(-n2c3ccccc3c3c4ccccc4ccc32)cc1 ; c1ccc(Nc2ccc(-c3cc4ccccc4c4ccccc34)cc2)cc1 ; c1ccc(N(c2ccc(-c3cc4ccccc4c4ccccc34)cc2)c2ccc(-n3c4ccccc4c4c5ccccc5ccc43)cc2)cc1]

subA-1 (10 g, 30.5 mmol), amine2 (11.1 g, 32 mmol), and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 10.1 g of Compound 2-2 (yield 52%, MS: [M+H]+=637).

### Preparation Example 2-3

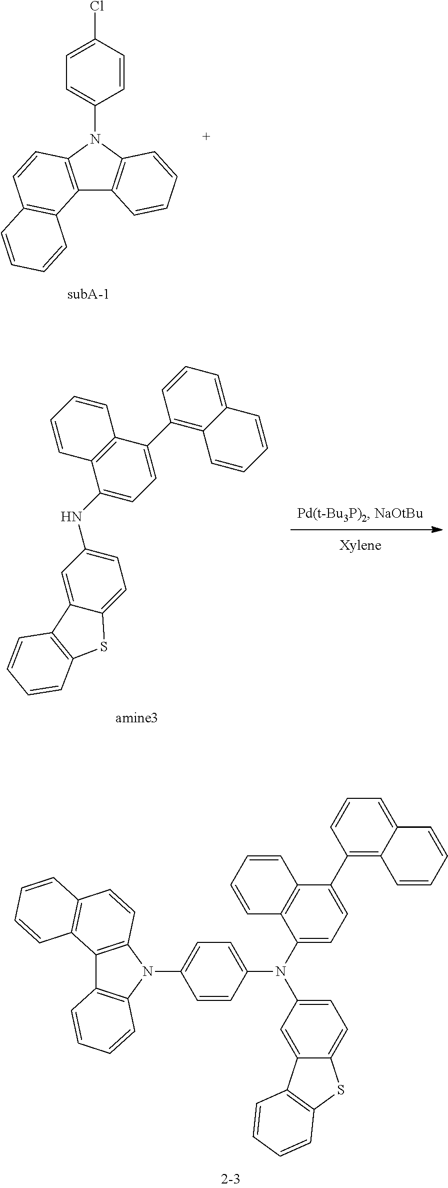 [SMILES: Clc1ccc(-n2c3ccccc3c3c4ccccc4ccc32)cc1 ; c1ccc2c(-c3ccc(Nc4ccc5sc6ccccc6c5c4)c4ccccc34)cccc2c1 ; c1ccc2c(-c3ccc(N(c4ccc(-n5c6ccccc6c6c7ccccc7ccc65)cc4)c4ccc5sc6ccccc6c5c4)c4ccccc34)cccc2c1]

subA-1 (10 g, 30.5 mmol), amine3 (14.5 g, 32 mmol), and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 14.7 g of Compound 2-3 (yield 65%, MS: [M+H]+=743).

### Preparation Example 2-4

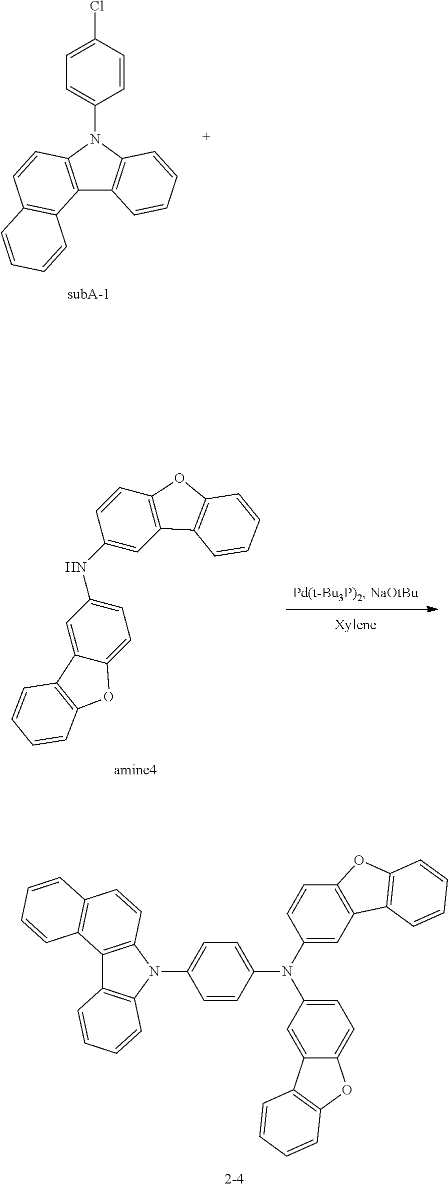 [SMILES: Clc1ccc(-n2c3ccccc3c3c4ccccc4ccc32)cc1 ; c1ccc2c(c1)oc1ccc(Nc3ccc4oc5ccccc5c4c3)cc12 ; c1ccc2c(c1)ccc1c2c2ccccc2n1-c1ccc(N(c2ccc3oc4ccccc4c3c2)c2ccc3oc4ccccc4c3c2)cc1]

subA-1 (10 g, 30.5 mmol), amine4 (11.2 g, 32 mmol), and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12.1 g of Compound 2-4 (yield 62%, MS: [M+H]+=641).

### Preparation Example 2-5

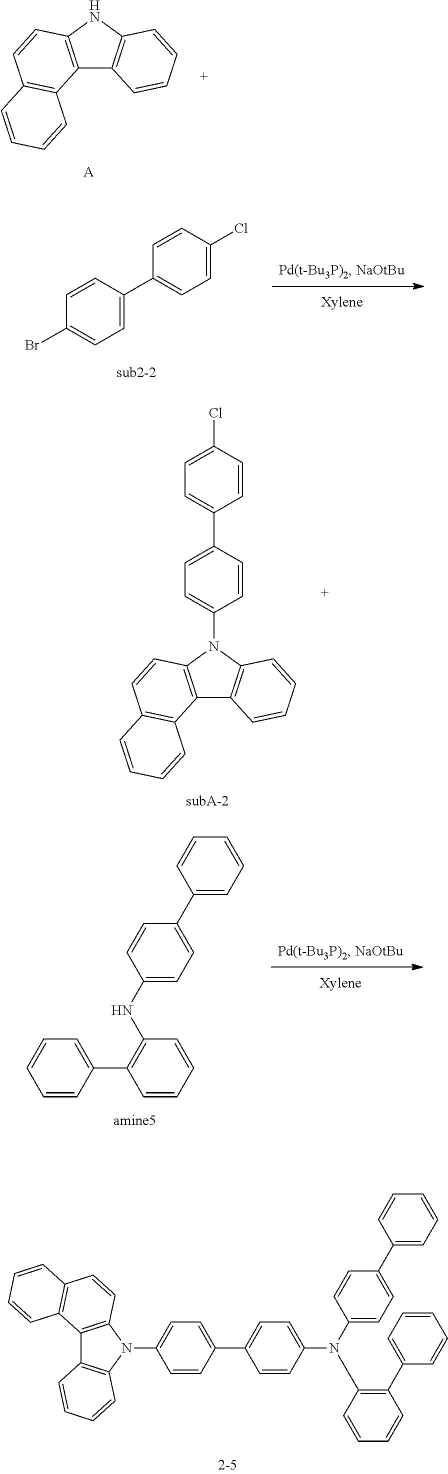

Compound A (10 g, 46 mmol), sub2-2 (12.9 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.1 g of subA-2 (yield 60%, MS: [M+H]+=404).

subA-2 (10 g, 37.4 mmol), amine5 (12.6 g, 39.2 mmol), and sodium tert-butoxide (4.7 g, 48.6 mmol) were added to 200 ml of xylene under a nitrogen atmosphere, and the mixture was stirred and refluxed. Then, bis(tri-tert-butylphosphine)palladium(0) (0.4 g, 0.7 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 15.7 g of Compound 2-5 (yield 61%, MS: [M+H]+=689).

### Preparation Example 2-6

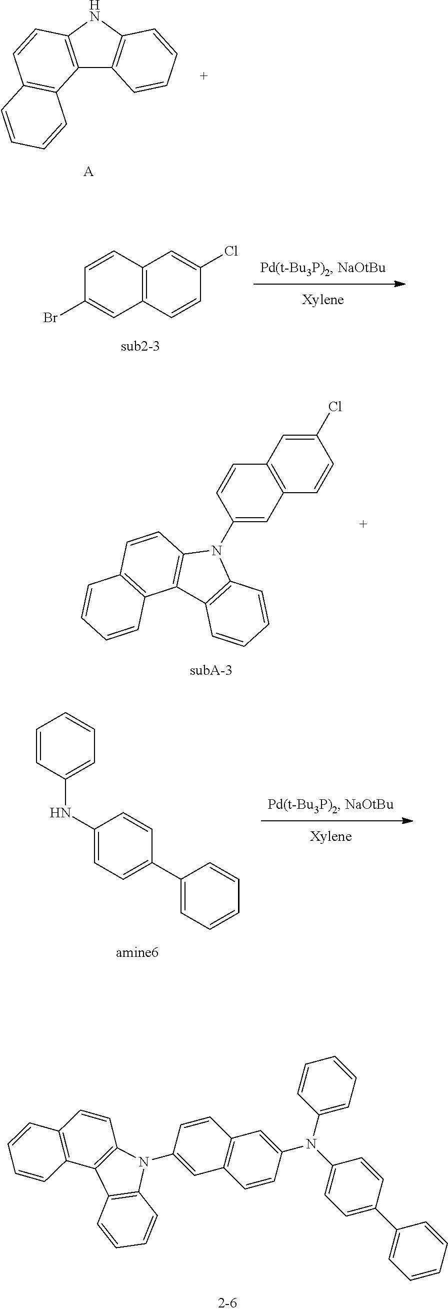

Compound A (10 g, 46 mmol), sub2-3 (11.7 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 9.7 g of subA-3 (yield 56%, MS: [M+H]+=378).

subA-3 (10 g, 26.5 mmol), amine6 (6.8 g, 27.8 mmol), and sodium tert-butoxide (3.3 g, 34.4 mmol) were added to 200 ml of xylene under a nitrogen atmosphere, and the mixture was stirred and refluxed. Then, bis(tri-tert-butylphosphine)palladium(0) (0.3 g, 0.5 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 8.1 g of Compound 2-6 (yield 52%, MS: [M+H]+=587).

### Preparation Example 2-7

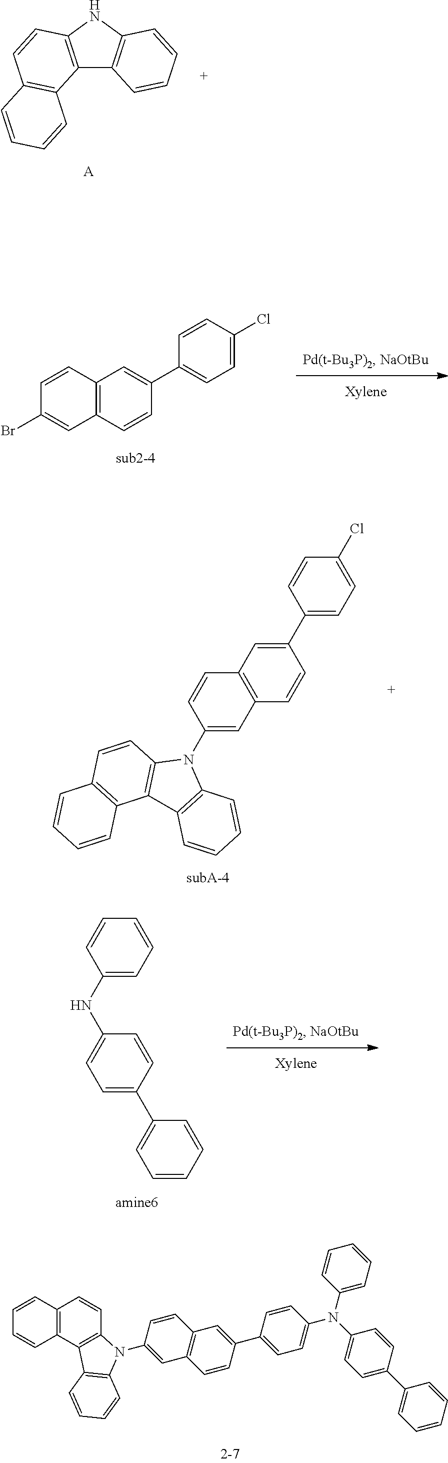

Compound A (10 g, 46 mmol), sub2-4 (15.3 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 14.6 g of subA-4 (yield 70%, MS: [M+H]+=454).

subA-4 (10 g, 22 mmol), amine6 (5.7 g, 23.1 mmol), and sodium tert-butoxide (2.8 g, 28.6 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 7.9 g of Compound 2-7 (yield 54%, MS: [M+H]+=663).

### Preparation Example 2-8

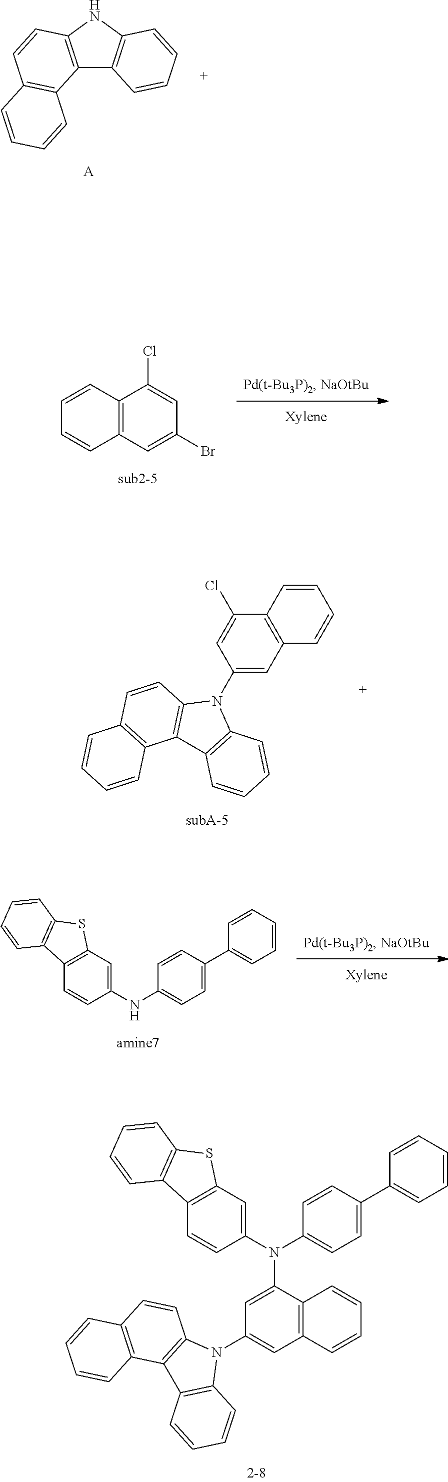

Compound A (10 g, 46 mmol), sub2-5 (11.7 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 8.7 g of subA-5 (yield 50%, MS: [M+H]+=378).

subA-5 (10 g, 26.5 mmol), amine7 (9.8 g, 27.8 mmol), and sodium tert-butoxide (3.3 g, 34.4 mmol) were added to 200 ml of xylene under a nitrogen atmosphere, and the mixture was stirred and refluxed. Then, bis(tri-tert-butylphosphine)palladium(0) (0.3 g, 0.5 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.7 g of Compound 2-8 (yield 64%, MS: [M+H]+=693).

### Preparation Example 2-9

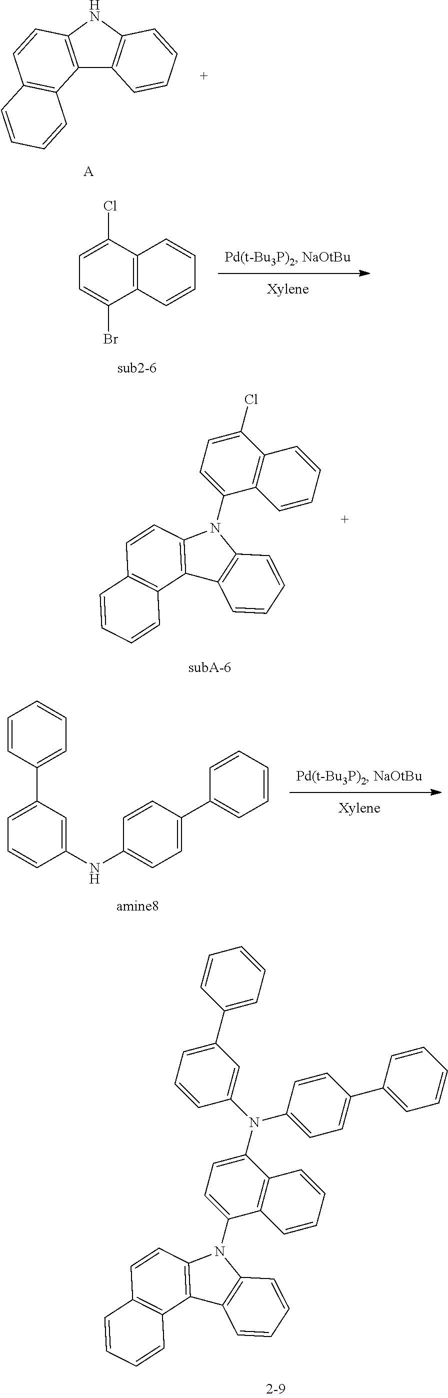

Compound A (10 g, 46 mmol), sub2-6 (11.7 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 8.7 g of subA-6 (yield 50%, MS: [M+H]+=378).

subA-6 (10 g, 26.5 mmol), amine8 (8.9 g, 27.8 mmol), and sodium tert-butoxide (3.3 g, 34.4 mmol) were added to 200 ml of xylene under a nitrogen atmosphere, and the mixture was stirred and refluxed. Then, bis(tri-tert-butylphosphine)palladium(0) (0.3 g, 0.5 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 9.5 g of Compound 2-9 (yield 54%, MS: [M+H]+=663).

### Preparation Example 2-10

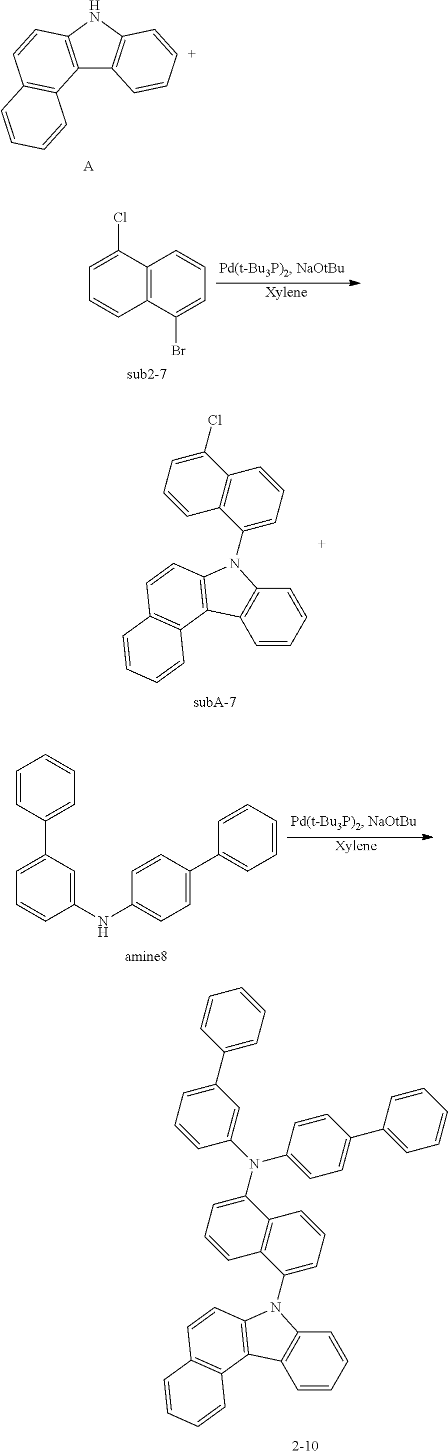

Compound A (10 g, 46 mmol), sub2-7 (11.7 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.1 g of subA-7 (yield 64%, MS: [M+H]+=378).

subA-7 (10 g, 26.5 mmol), amine8 (8.9 g, 27.8 mmol), and sodium tert-butoxide (3.3 g, 34.4 mmol) were added to 200 ml of xylene under a nitrogen atmosphere, and the mixture was stirred and refluxed. Then, bis(tri-tert-butylphosphine)palladium(0) (0.3 g, 0.5 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 10.7 g of Compound 2-10 (yield 61%, MS: [M+H]+=663).

### Preparation Example 2-11

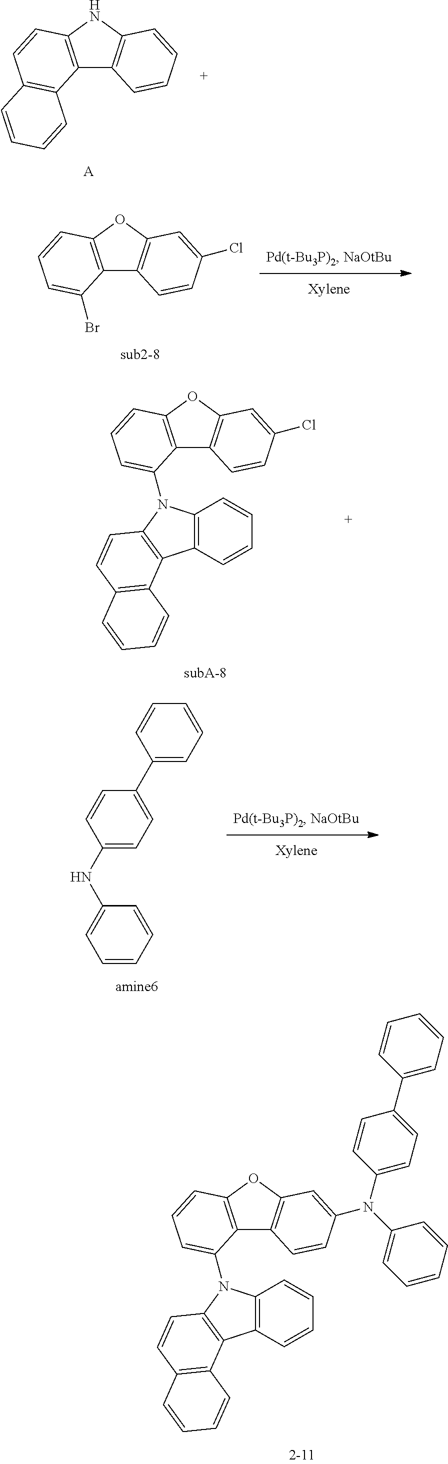

Compound A (10 g, 46 mmol), sub2-8 (13.6 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.1 g of subA-8 (yield 68%, MS: [M+H]+=418).

subA-8 (10 g, 23.9 mmol), amine6 (6.2 g, 25.1 mmol), and sodium tert-butoxide (3 g, 31.1 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 7.9 g of Compound 2-11 (yield 53%, MS: [M+H]+=627).

### Preparation Example 2-12

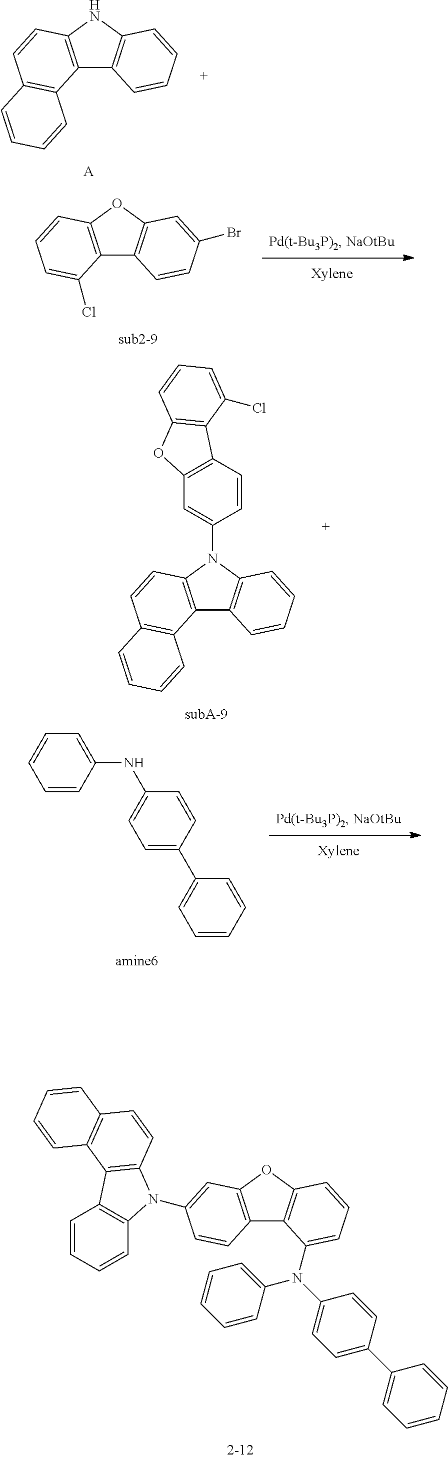

Compound A (10 g, 46 mmol), sub2-9 (13.6 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.1 g of subA-9 (yield 68%, MS: [M+H]+=418).

subA-9 (10 g, 23.9 mmol), amine6 (6.2 g, 25.1 mmol), and sodium tert-butoxide (3 g, 31.1 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 9.4 g of Compound 2-12 (yield 63%, MS: [M+H]+=627).

### Preparation Example 2-13

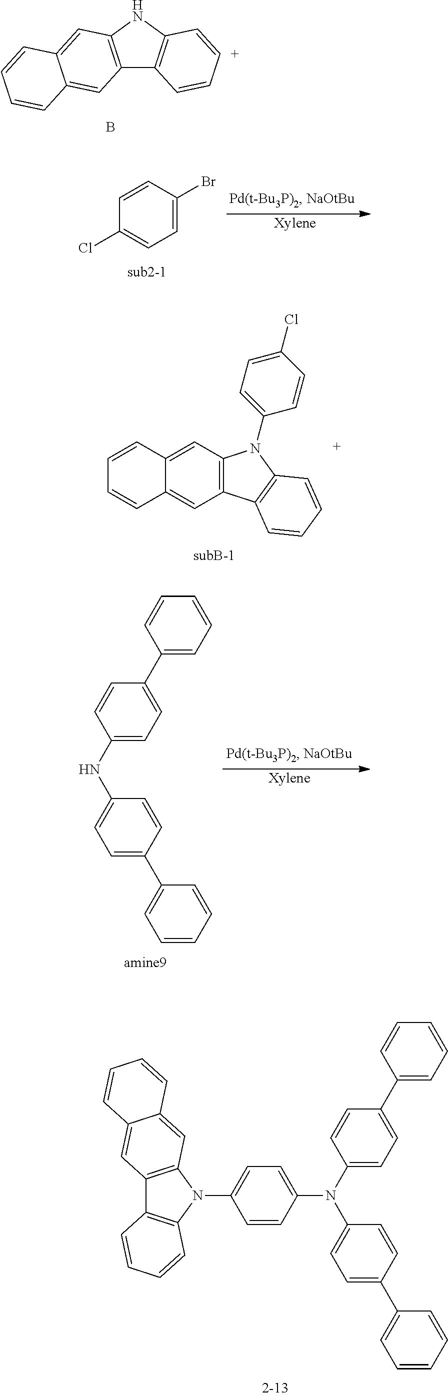

Compound B (10 g, 46 mmol), sub2-1 (9.3 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 8.7 g of subB-1 (yield 58%, MS: [M+H]+=328).

subB-1 (10 g, 30.5 mmol), amine9 (10.3 g, 32 mmol), and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12.9 g of Compound 2-13 (yield 69%, MS: [M+H]+=613).

### Preparation Example 2-14

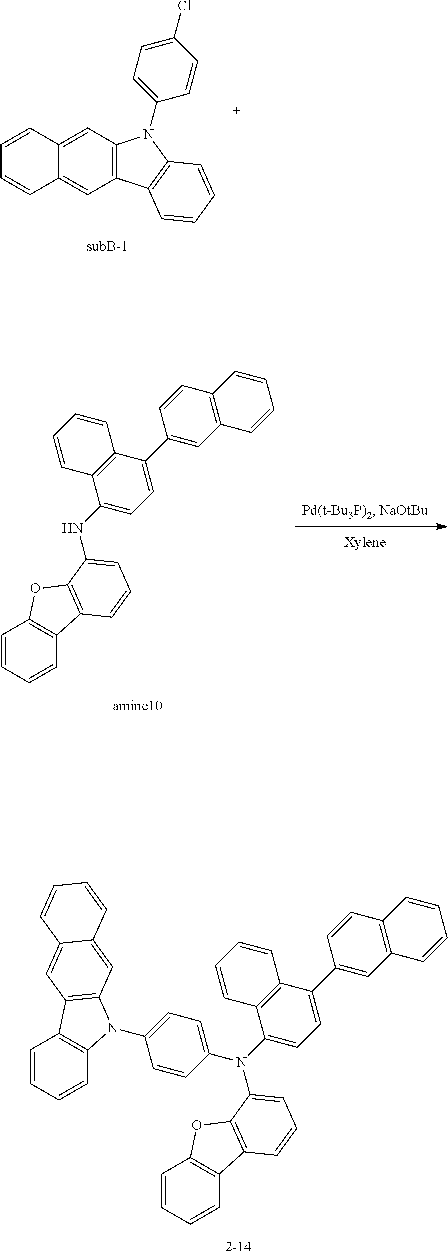 [SMILES: Clc1ccc(-n2c3ccccc3c3cc4ccccc4cc32)cc1 ; c1ccc2cc(-c3ccc(Nc4cccc5c4oc4ccccc45)c4ccccc34)ccc2c1 ; c1ccc2cc(-c3ccc(N(c4ccc(-n5c6ccccc6c6cc7ccccc7cc65)cc4)c4cccc5c4oc4ccccc45)c4ccccc34)ccc2c1]

subB-1 (10 g, 30.5 mmol), amine10 (14 g, 32 mmol), and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12.6 g of Compound 2-14 (yield 57%, MS: [M+H]+=727).

### Preparation Example 2-15

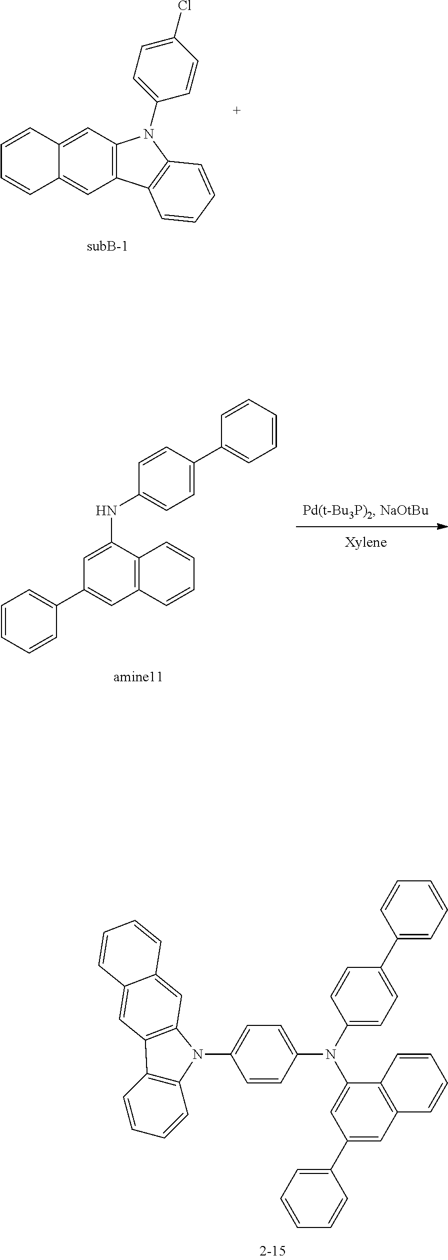 [SMILES: Clc1ccc(-n2c3ccccc3c3cc4ccccc4cc32)cc1 ; c1ccc(-c2ccc(Nc3cc(-c4ccccc4)cc4ccccc34)cc2)cc1 ; c1ccc(-c2ccc(N(c3ccc(-n4c5ccccc5c5cc6ccccc6cc54)cc3)c3cc(-c4ccccc4)cc4ccccc34)cc2)cc1]

subB-1 (10 g, 30.5 mmol), amine11 (11.9 g, 32 mmol), and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.4 g of Compound 2-15 (yield 66%, MS: [M+H]+=668).

### Preparation Example 2-16

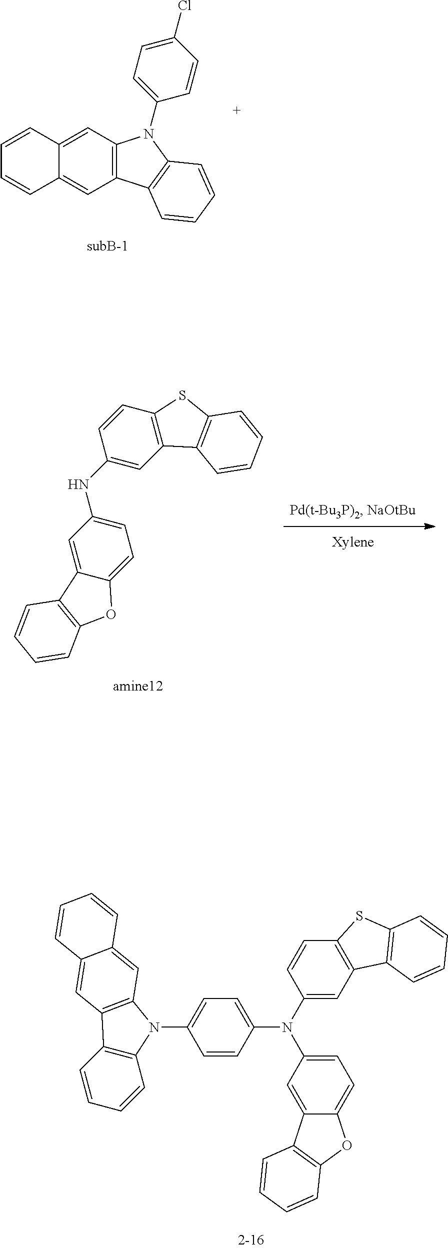 [SMILES: Clc1ccc(-n2c3ccccc3c3cc4ccccc4cc32)cc1 ; c1ccc2c(c1)oc1ccc(Nc3ccc4sc5ccccc5c4c3)cc12 ; c1ccc2cc3c(cc2c1)c1ccccc1n3-c1ccc(N(c2ccc3oc4ccccc4c3c2)c2ccc3sc4ccccc4c3c2)cc1]

subB-1 (10 g, 30.5 mmol), amine12 (11.7 g, 32 mmol), and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12 g of Compound 2-16 (yield 60%, MS: [M+H]+=657).

### Preparation Example 2-17

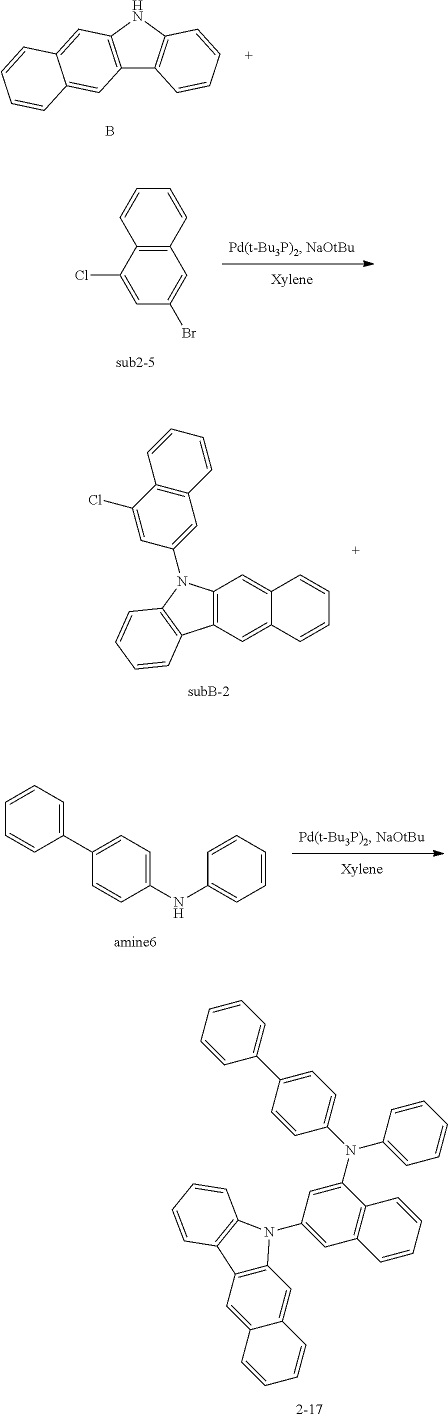

Compound B (10 g, 46 mmol), sub2-5 (11.7 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 9.9 g of subB-2 (yield 57%, MS: [M+H]+=378).

subB-2 (10 g, 26.5 mmol), amine6 (6.8 g, 27.8 mmol), and sodium tert-butoxide (3.3 g, 34.4 mmol) were added to 200 ml of xylene under a nitrogen atmosphere, and the mixture was stirred and refluxed. Then, bis(tri-tert-butylphosphine)palladium(0) (0.3 g, 0.5 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 8.7 g of Compound 2-17 (yield 56%, MS: [M+H]+=587).

### Preparation Example 2-18

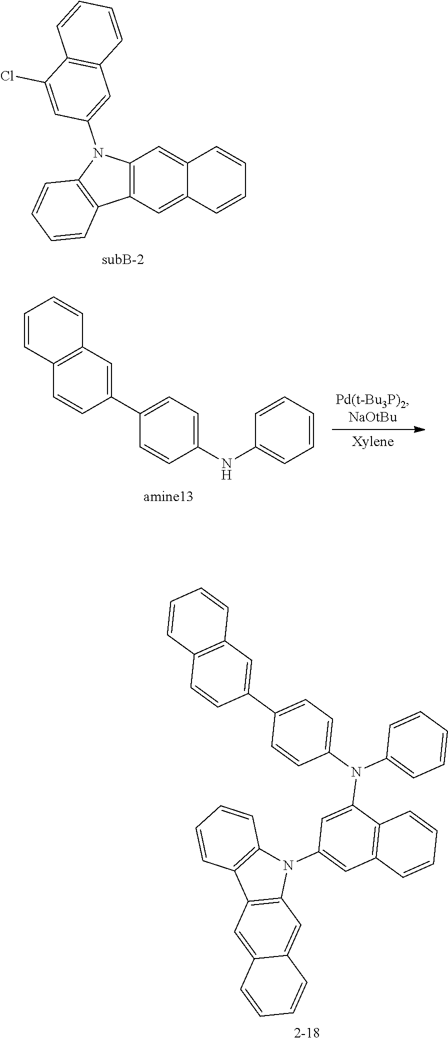 [SMILES: Clc1cc(-n2c3ccccc3c3cc4ccccc4cc32)cc2ccccc12 ; c1ccc(Nc2ccc(-c3ccc4ccccc4c3)cc2)cc1 ; c1ccc(N(c2ccc(-c3ccc4ccccc4c3)cc2)c2cc(-n3c4ccccc4c4cc5ccccc5cc43)cc3ccccc23)cc1]

subB-2 (10 g, 26.5 mmol), amine13 (8.2 g, 27.8 mmol), and sodium tert-butoxide (3.3 g, 34.4 mmol) were added to 200 ml of xylene under a nitrogen atmosphere, and the mixture was stirred and refluxed. Then, bis(tri-tert-butylphosphine)palladium(0) (0.3 g, 0.5 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 8.9 g of Compound 2-18 (yield 53%, MS: [M+H]+=637).

### Preparation Example 2-19

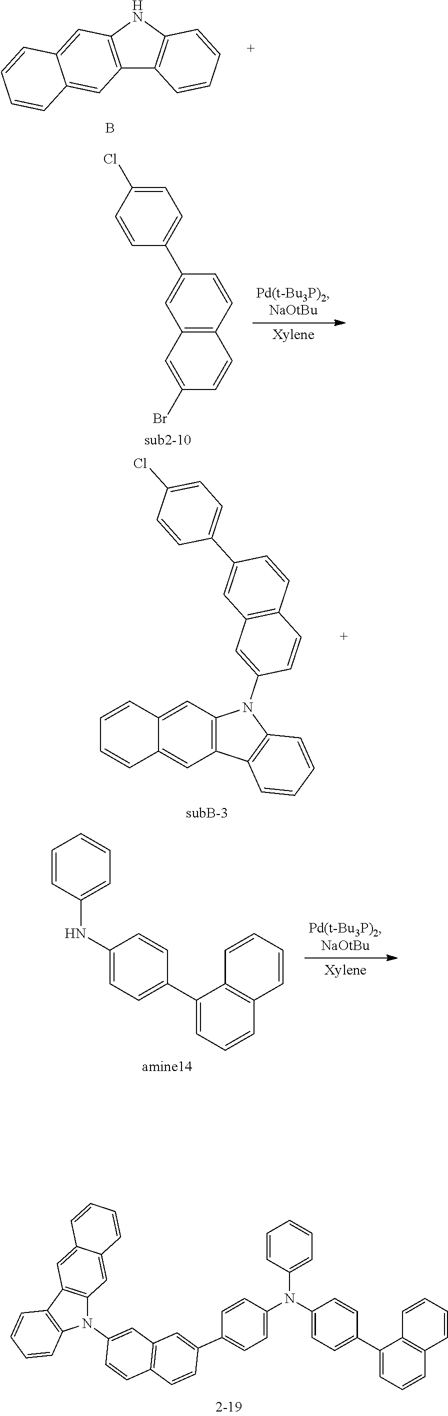

Compound B (10 g, 46 mmol), sub2-10 (15.3 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12.1 g of subB-3 (yield 58%, MS: [M+H]+=454).

subB-3 (10 g, 22 mmol), amine14 (6.8 g, 23.1 mmol), and sodium tert-butoxide (2.8 g, 28.6 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 10.8 g of Compound 2-19 (yield 69%, MS: [M+H]+=713).

### Preparation Example 2-20

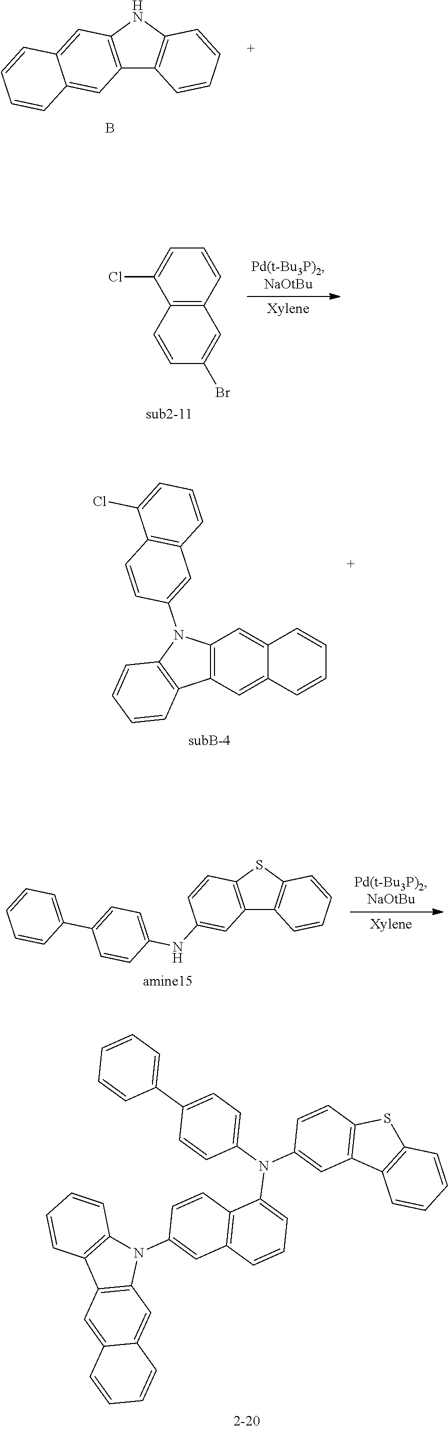

Compound B (10 g, 46 mmol), sub2-11 (11.7 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 10.2 g of subB-4 (yield 59%, MS: [M+H]+=378).

subB-4 (10 g, 26.5 mmol), amine15 (9.8 g, 27.8 mmol), and sodium tert-butoxide (3.3 g, 34.4 mmol) were added to 200 ml of xylene under a nitrogen atmosphere, and the mixture was stirred and refluxed. Then, bis(tri-tert-butylphosphine)palladium(0) (0.3 g, 0.5 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.4 g of Compound 2-20 (yield 62%, MS: [M+H]+=693).

### Preparation Example 2-21

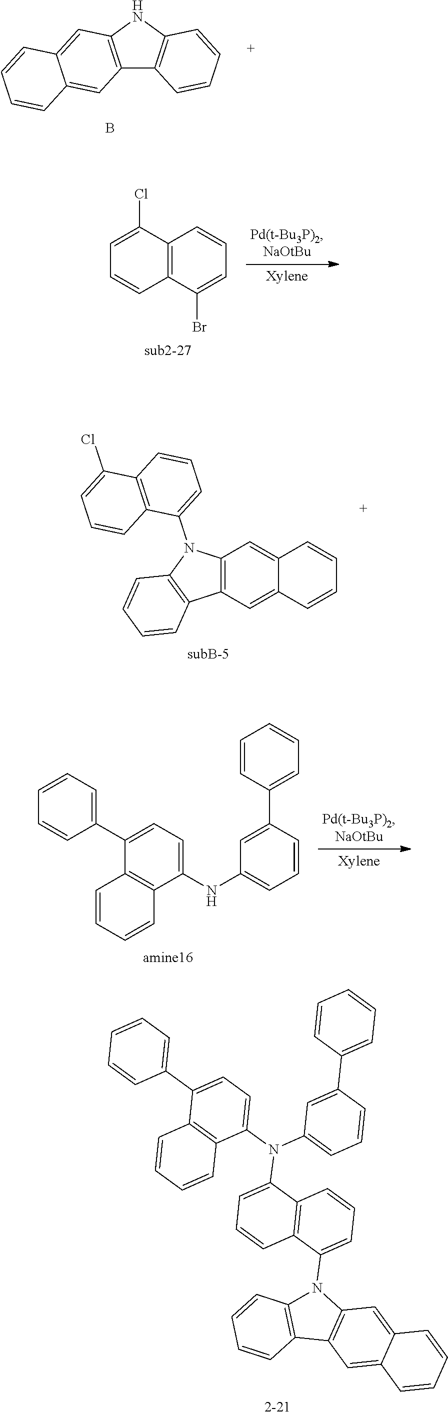

Compound B (10 g, 46 mmol), sub2-7 (11.7 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 9.5 g of subB-5 (yield 55%, MS: [M+H]+=378).

subB-5 (10 g, 26.5 mmol), amine16 (10.3 g, 27.8 mmol), and sodium tert-butoxide (3.3 g, 34.4 mmol) were added to 200 ml of xylene under a nitrogen atmosphere, and the mixture was stirred and refluxed. Then, bis(tri-tert-butylphosphine)palladium(0) (0.3 g, 0.5 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.2 g of Compound 2-21 (yield 70%, MS: [M+H]+=713).

### Preparation Example 2-22

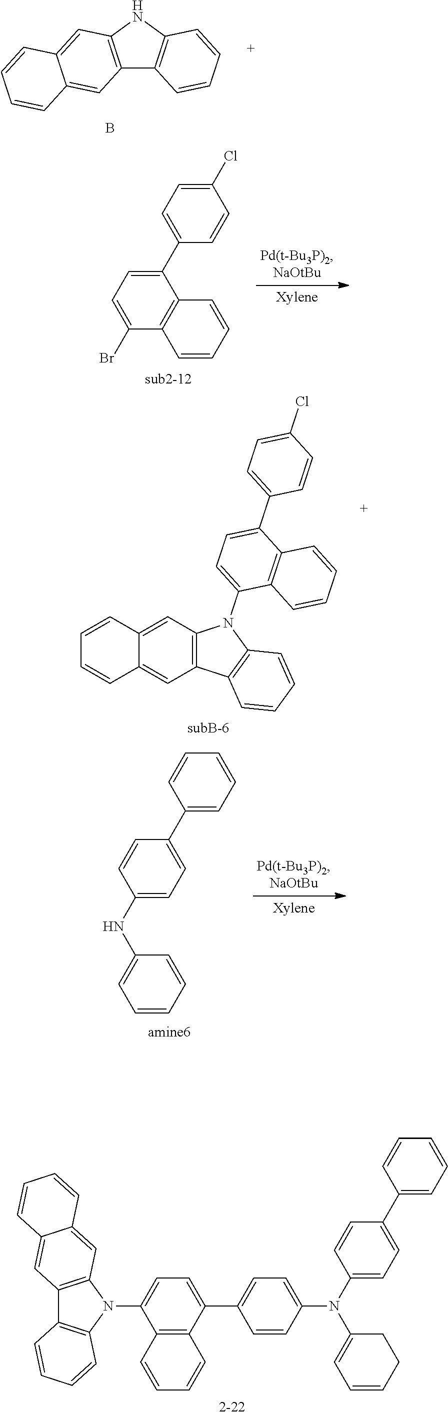

Compound B (10 g, 46 mmol), sub2-12 (15.3 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 14.6 g of subB-6 (yield 70%, MS: [M+H]+=454).

subB-6 (10 g, 22 mmol), amine6 (5.7 g, 23.1 mmol), and sodium tert-butoxide (2.8 g, 28.6 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 10.1 g of Compound 2-22 (yield 69%, MS: [M+H]+=665).

### Preparation Example 2-23

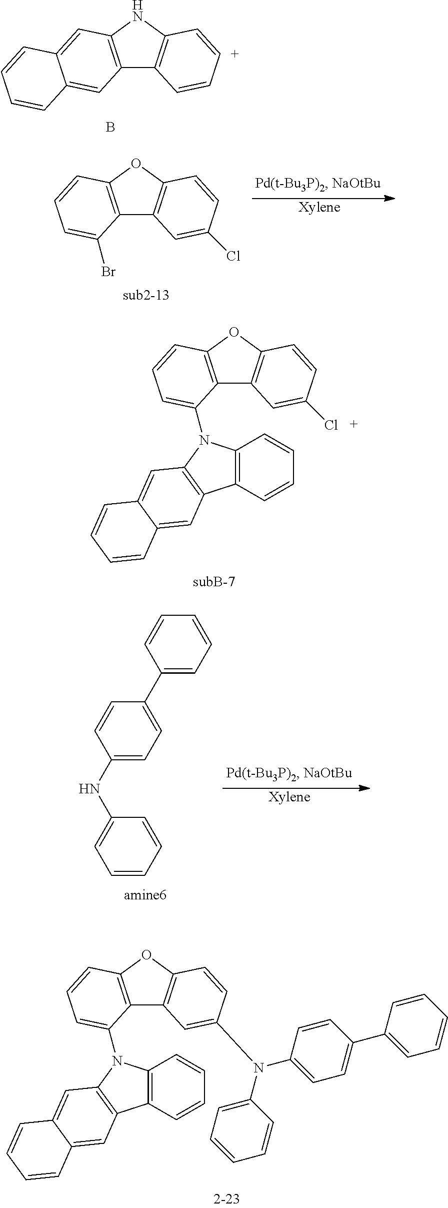

Compound B (10 g, 46 mmol), sub2-13 (13.6 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.4 g of subB-7 (yield 70%, MS: [M+H]+=418).

subB-7 (10 g, 23.9 mmol), amine6 (6.2 g, 25.1 mmol), and sodium tert-butoxide (3 g, 31.1 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 10 g of Compound 2-23 (yield 67%, MS: [M+H]+=627).

### Preparation Example 2-24

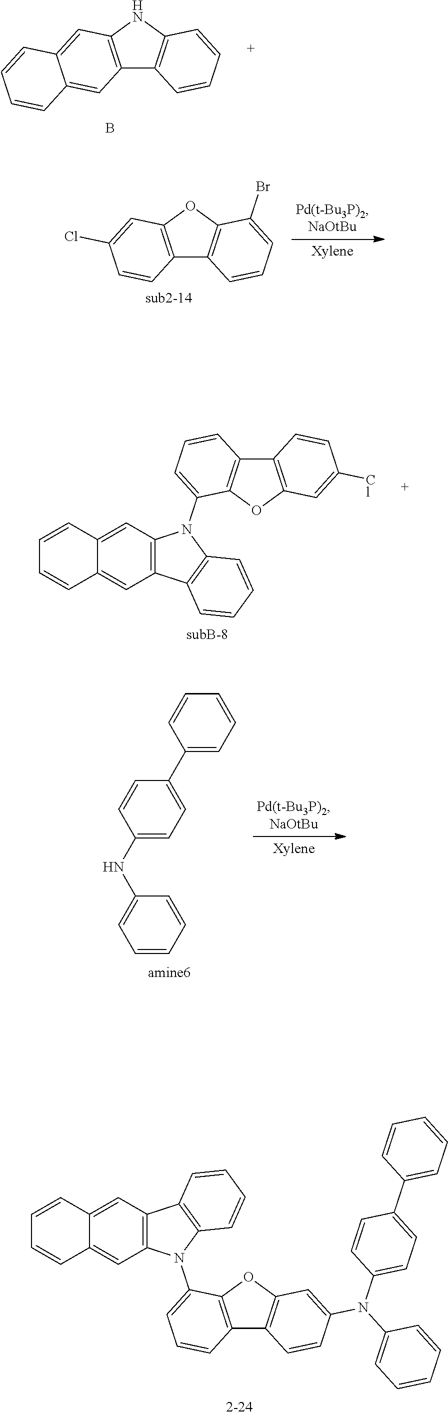

Compound B (10 g, 46 mmol), sub2-14 (13.6 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12.1 g of subB-8 (yield 63%, MS: [M+H]+=418).

subB-8 (10 g, 23.9 mmol), amine6 (6.2 g, 25.1 mmol), and sodium tert-butoxide (3 g, 31.1 mmol) were added to 200 ml of xylene under a nitrogen atmosphere, and the mixture was stirred and refluxed. Then, bis(tri-tert-butylphosphine)palladium(0) (0.2 g, 0.5 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 9.9 g of Compound 2-24 (yield 66%, MS: [M+H]+=627).

### Preparation Example 2-25

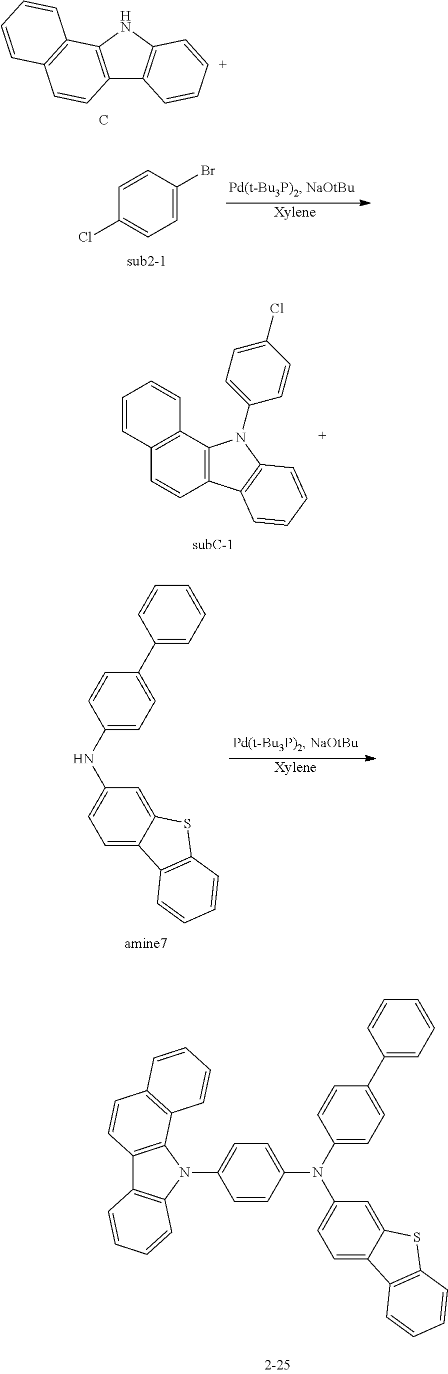

Compound C (10 g, 46 mmol), sub2-1 (9.3 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 9.8 g of subC-1 (yield 65%, MS: [M+H]+=328).

subC-1 (10 g, 30.5 mmol), amine7 (11.3 g, 32 mmol), and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12.9 g of Compound 2-25 (yield 66%, MS: [M+H]+=643).

### Preparation Example 2-26

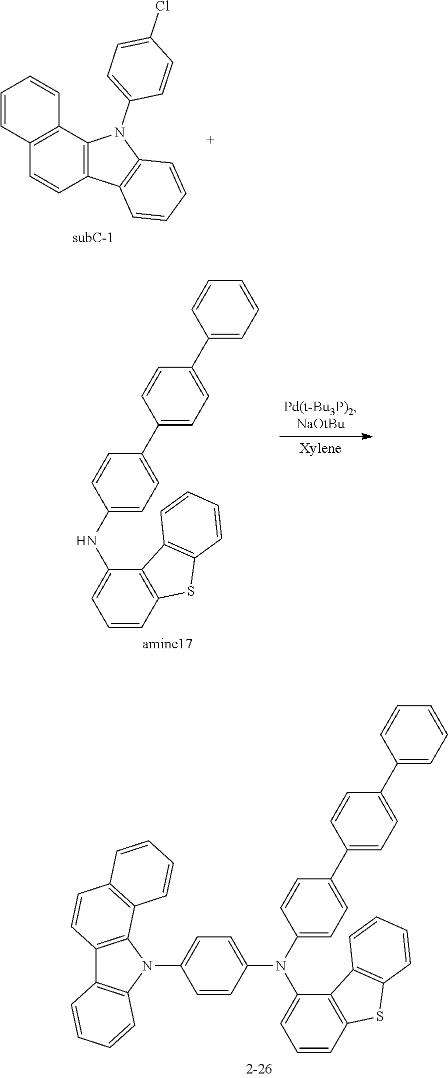 [SMILES: Clc1ccc(-n2c3ccccc3c3ccc4ccccc4c32)cc1 ; "subC-1" ; c1ccc(-c2ccc(-c3ccc(Nc4cccc5sc6ccccc6c45)cc3)cc2)cc1 ; c1ccc(-c2ccc(-c3ccc(N(c4ccc(-n5c6ccccc6c6ccc7ccccc7c65)cc4)c4cccc5sc6ccccc6c45)cc3)cc2)cc1]

subC-1 (10 g, 30.5 mmol), amine17 (13.7 g, 32 mmol), and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 14.7 g of Compound 2-26 (yield 67%, MS: [M+H]+=719).

### Preparation Example 2-27

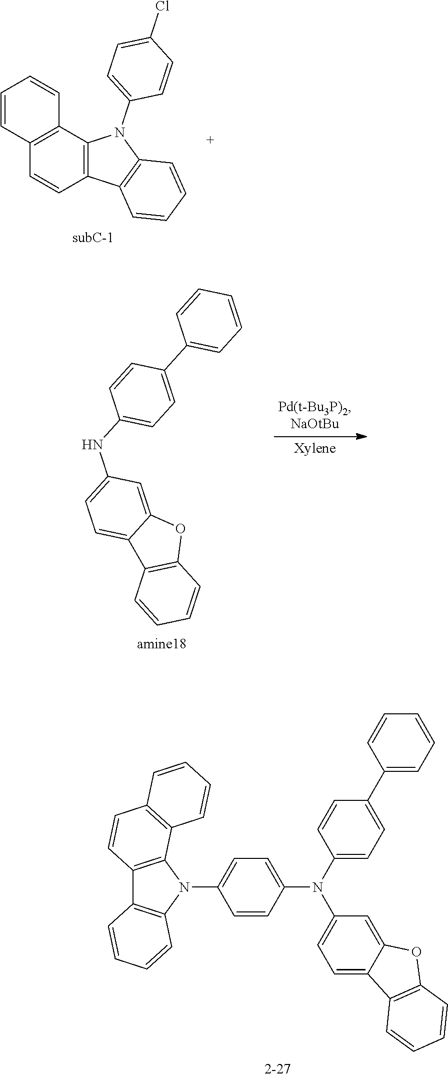 [SMILES: Clc1ccc(-n2c3ccccc3c3ccc4ccccc4c32)cc1 ; "subC-1" ; c1ccc(-c2ccc(Nc3ccc4c(c3)oc3ccccc34)cc2)cc1 ; c1ccc(-c2ccc(N(c3ccc(-n4c5ccccc5c5ccc6ccccc6c54)cc3)c3ccc4c(c3)oc3ccccc34)cc2)cc1]

subC-1 (10 g, 30.5 mmol), amine18 (10.7 g, 32 mmol), and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12 g of Compound 2-27 (yield 63%, MS: [M+H]+=627).

### Preparation Example 2-28

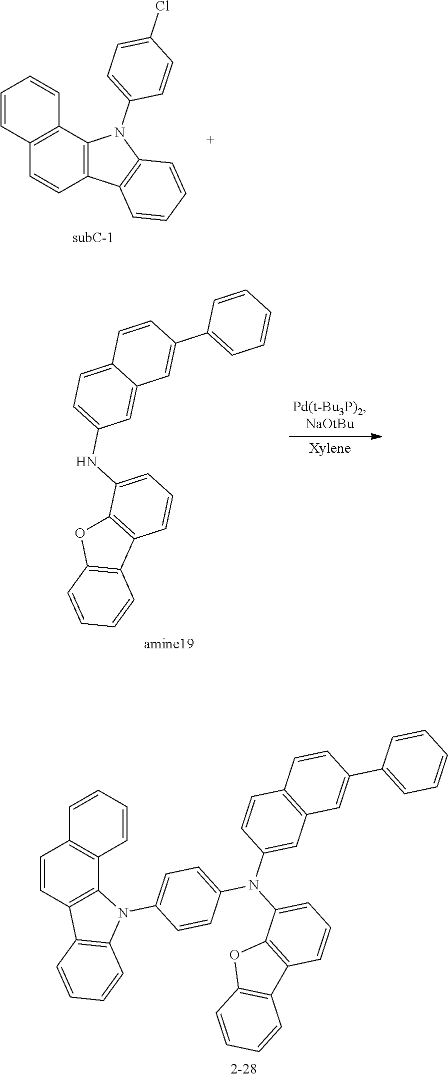 [SMILES: Clc1ccc(-n2c3ccccc3c3ccc4ccccc4c32)cc1 ; "subC-1" ; c1ccc(-c2ccc3ccc(Nc4cccc5c4oc4ccccc45)cc3c2)cc1 ; c1ccc(-c2ccc3ccc(N(c4ccc(-n5c6ccccc6c6ccc7ccccc7c65)cc4)c4cccc5c4oc4ccccc45)cc3c2)cc1]

subC-1 (10 g, 30.5 mmol), amine19 (12.3 g, 32 mmol), and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.2 g of Compound 2-28 (yield 64%, MS: [M+H]+=677).

### Preparation Example 2-29

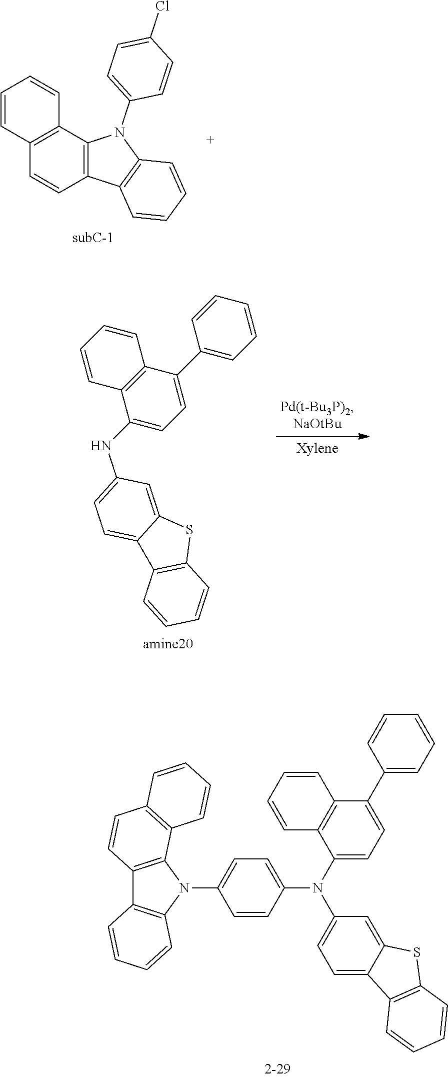 [SMILES: Clc1ccc(-n2c3ccccc3c3ccc4ccccc4c32)cc1 ; "subC-1" ; c1ccc(-c2ccc(Nc3ccc4c(c3)sc3ccccc34)c3ccccc23)cc1 ; c1ccc(-c2ccc(N(c3ccc(-n4c5ccccc5c5ccc6ccccc6c54)cc3)c3ccc4c(c3)sc3ccccc34)c3ccccc23)cc1]

subC-1 (10 g, 30.5 mmol), amine20 (12.9 g, 32 mmol), and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 14.6 g of Compound 2-29 (yield 69%, MS: [M+H]+=693).

### Preparation Example 2-30

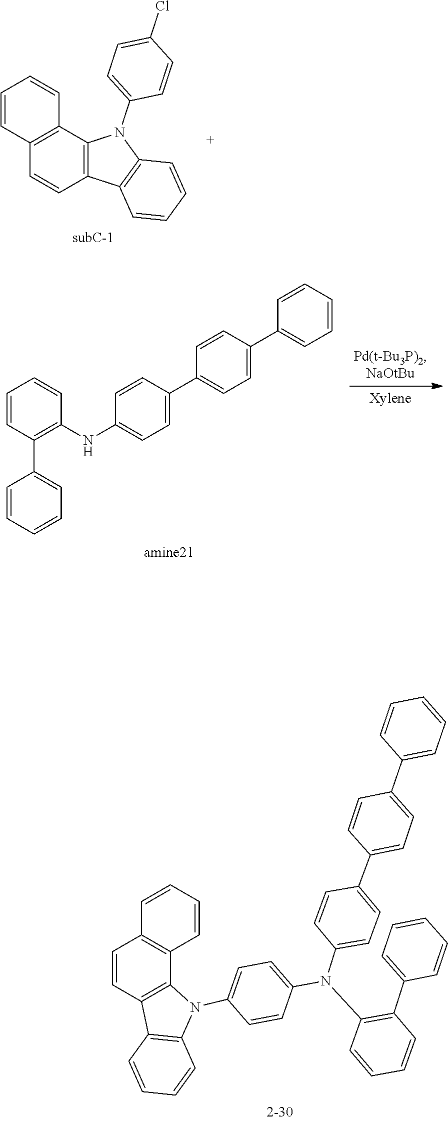 [SMILES: Clc1ccc(-n2c3ccccc3c3ccc4ccccc4c32)cc1 ; "subC-1" ; c1ccc(-c2ccc(-c3ccc(Nc4ccccc4-c4ccccc4)cc3)cc2)cc1 ; c1ccc(-c2ccc(-c3ccc(N(c4ccc(-n5c6ccccc6c6ccc7ccccc7c65)cc4)c4ccccc4-c4ccccc4)cc3)cc2)cc1]

subC-1 (10 g, 30.5 mmol), amine21 (12.7 g, 32 mmol), and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.6 g of Compound 2-30 (yield 65%, MS: [M+H]+=689).

### Preparation Example 2-31

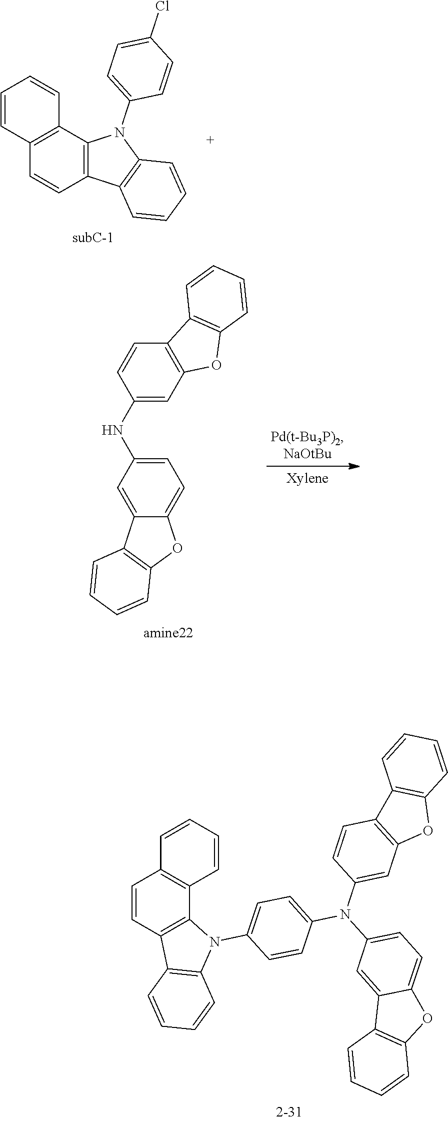 [SMILES: Clc1ccc(-n2c3ccccc3c3ccc4ccccc4c32)cc1 ; "subC-1" ; c1ccc2c(c1)oc1cc(Nc3ccc4oc5ccccc5c4c3)ccc12 ; c1ccc2c(c1)ccc1c3ccccc3n(-c3ccc(N(c4ccc5c(c4)oc4ccccc45)c4ccc5oc6ccccc6c5c4)cc3)c21]

subC-1 (10 g, 30.5 mmol), amine22 (11.2 g, 32 mmol), and sodium tert-butoxide (3.8 g, 39.7 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 10.2 g of Compound 2-31 (yield 52%, MS: [M+H]+=641).

### Preparation Example 2-32

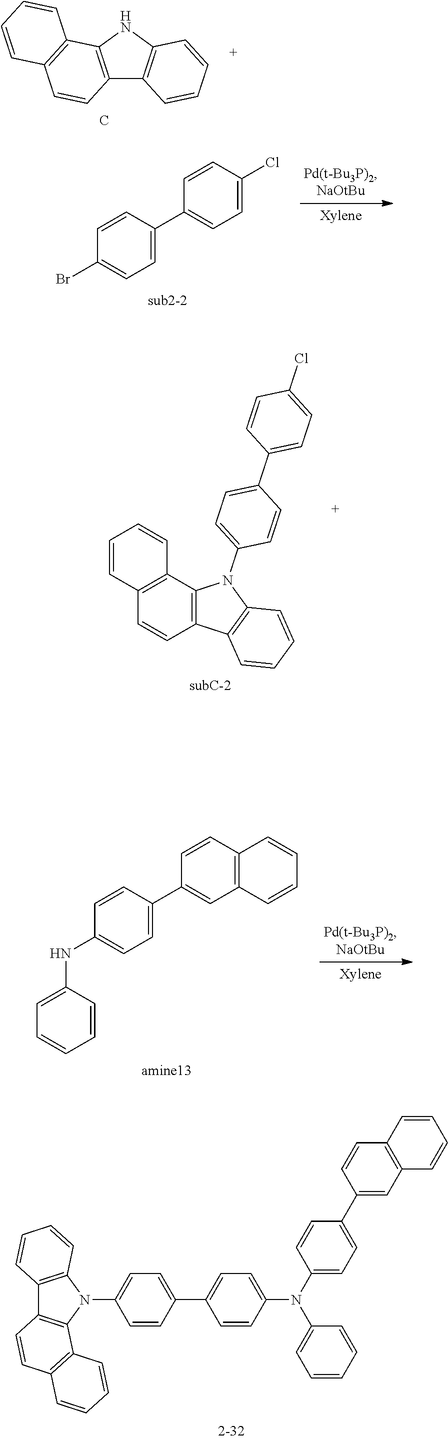

Compound C (10 g, 46 mmol), sub2-2 (12.9 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.7 g of subC-2 (yield 63%, MS: [M+H]+=404).

subC-2 (10 g, 24.8 mmol), amine13 (7.7 g, 26 mmol), and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.3 g, 0.5 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 10.2 g of Compound 2-32 (yield 62%, MS: [M+H]+=663).

### Preparation Example 2-33

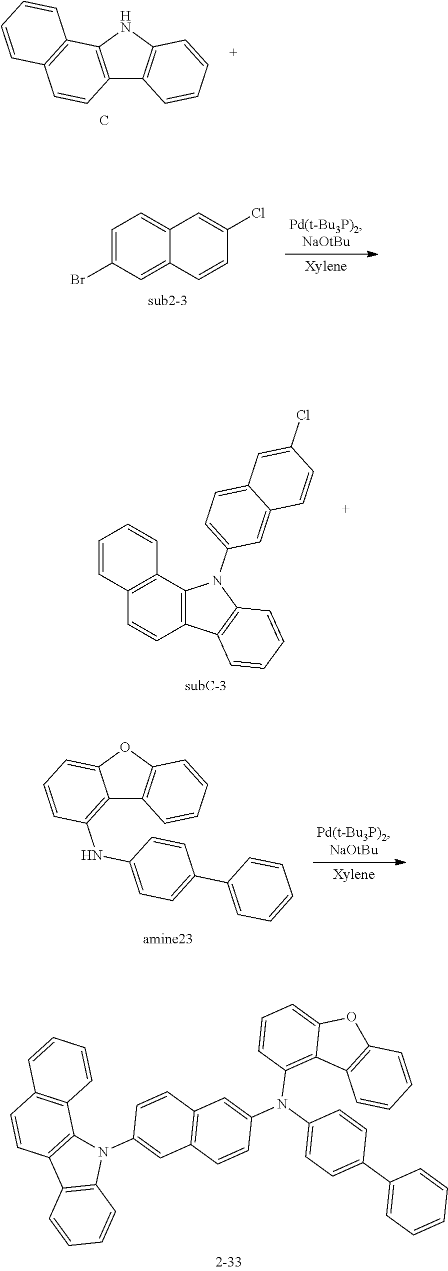

Compound C (10 g, 46 mmol), sub2-3 (11.7 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 10.9 g of subC-3 (yield 63%, MS: [M+H]+=378).

subC-3 (10 g, 24.8 mmol), amine23 (8.7 g, 26 mmol), and sodium tert-butoxide (3.1 g, 32.2 mmol) were added to 200 ml of xylene under a nitrogen atmosphere, and the mixture was stirred and refluxed. Then, bis(tri-tert-butylphosphine)palladium(0) (0.3 g, 0.5 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 9 g of Compound 2-33 (yield 54%, MS: [M+H]+=677).

### Preparation Example 2-34

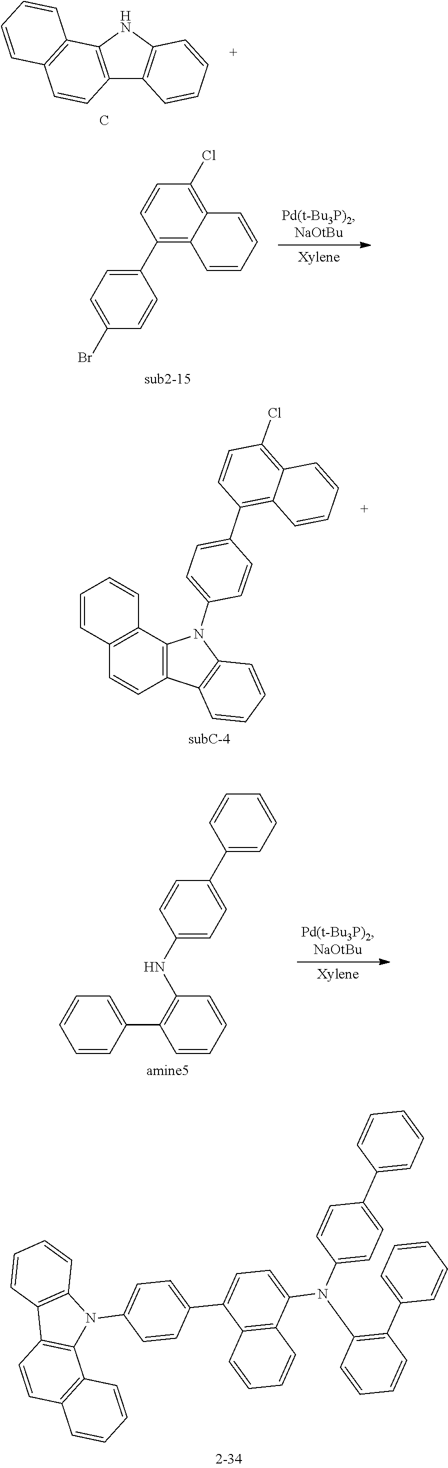

Compound C (10 g, 46 mmol), sub2-15 (15.3 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 3 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.6 g of subC-4 (yield 65%, MS: [M+H]+=454).

subC-4 (10 g, 22 mmol), amine5 (7.4 g, 23.1 mmol), and sodium tert-butoxide (2.8 g, 28.6 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 10.2 g of Compound 2-34 (yield 63%, MS: [M+H]+=739).

### Preparation Example 2-35

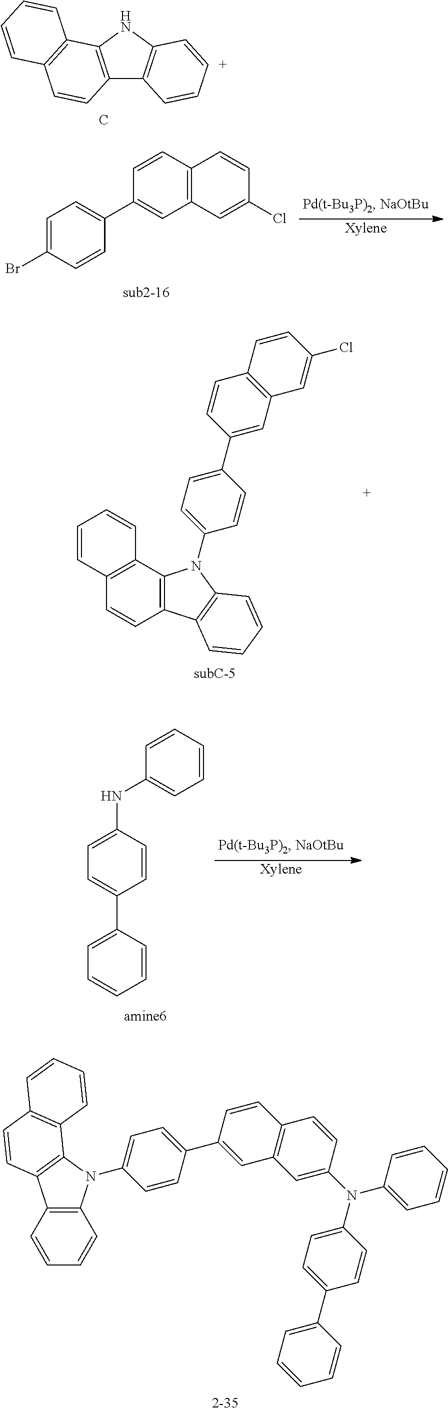

Compound C (10 g, 46 mmol), sub2-16 (12.9 g, 48.3 mmol), and sodium tert-butoxide (5.7 g, 59.8 mmol) were added to 200 ml of xylene under a nitrogen atmosphere, and the mixture was stirred and refluxed. Then, bis(tri-tert-butylphosphine)palladium(0) (0.5 g, 0.9 mmol) was added thereto. After 2 hours, the reaction was terminated, and then the solvent was removed by cooling to room temperature under reduced pressure. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 10.4 g of subC-5 (yield 50%, MS: [M+H]+=454).

subC-5 (10 g, 22 mmol), amine6 (5.7 g, 23.1 mmol), and sodium tert-butoxide (2.8 g, 28.6 mmol) were added to 200 ml of xylene under a nitrogen atmosphere, and the mixture was stirred and refluxed. Then, bis(tri-tert-butylphosphine)palladium(0) (0.2 g, 0.4 mmol) was added thereto. After 3 hours, the reaction was terminated, and then the solvent was removed by cooling to room temperature under reduced pressure. Then, the compound was completely dissolved again in chloroform, and washed twice with water.

Thereafter, the organic layer was separated, treated with anhydrous magnesium sulfate, then filtered, and the filtrate was distilled under reduced pressure. The concentrated compound was purified by silica gel column chromatography to prepare 8.5 g of Compound 2-35 (yield 58%, MS: [M+H]+=663).

### EXAMPLES

### Example 1

A glass substrate on which ITO (indium tin oxide) was coated as a thin film to a thickness of 1,000 Å was put into distilled water in which a detergent was dissolved, and ultrasonically cleaned. At this time, a product manufactured by Fischer Co. was used as the detergent, and distilled water filtered twice using a filter manufactured by Millipore Co. was used as the distilled water. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. The substrate was cleaned for 5 minutes using oxygen plasma and then transferred to a vacuum depositor.

A hole injection layer was formed on the prepared ITO transparent electrode to a thickness of 1150 Å with the following Compound HI-1, and the following Compound A-1 was p-doped at a concentration of 1.5%. Then, the following Compound HT-1 was vacuum-deposited on the hole injection layer to a thickness of 800 Å to form a hole transport layer. Thereafter, the following Compound EB-1 was vacuum-deposited on the hole transport layer to a thickness of 150 Å as an electron blocking layer. Then, Compound 1-2, Compound 2-1, and the following Compound Dp-7 were vacuum-deposited on the EB-1 deposited film to a thickness of 400 Å in a weight ratio of 49:49:2 to form a red light emitting layer. A hole blocking layer was formed by vacuum-depositing the following Compound HB-1 to a thickness of 30 Å on the light emitting layer. Then, the following Compound ET-1 and the following Compound LiQ were vacuum-deposited on the hole blocking layer to a thickness of 300 Å in a weight ratio of 2:1 to form an electron injection and transport layer. Lithium fluoride (LiF) and aluminum were sequentially deposited on the electron injection and transport layer to a thickness of 12 Å and 1,000 Å, respectively, to form a cathode.

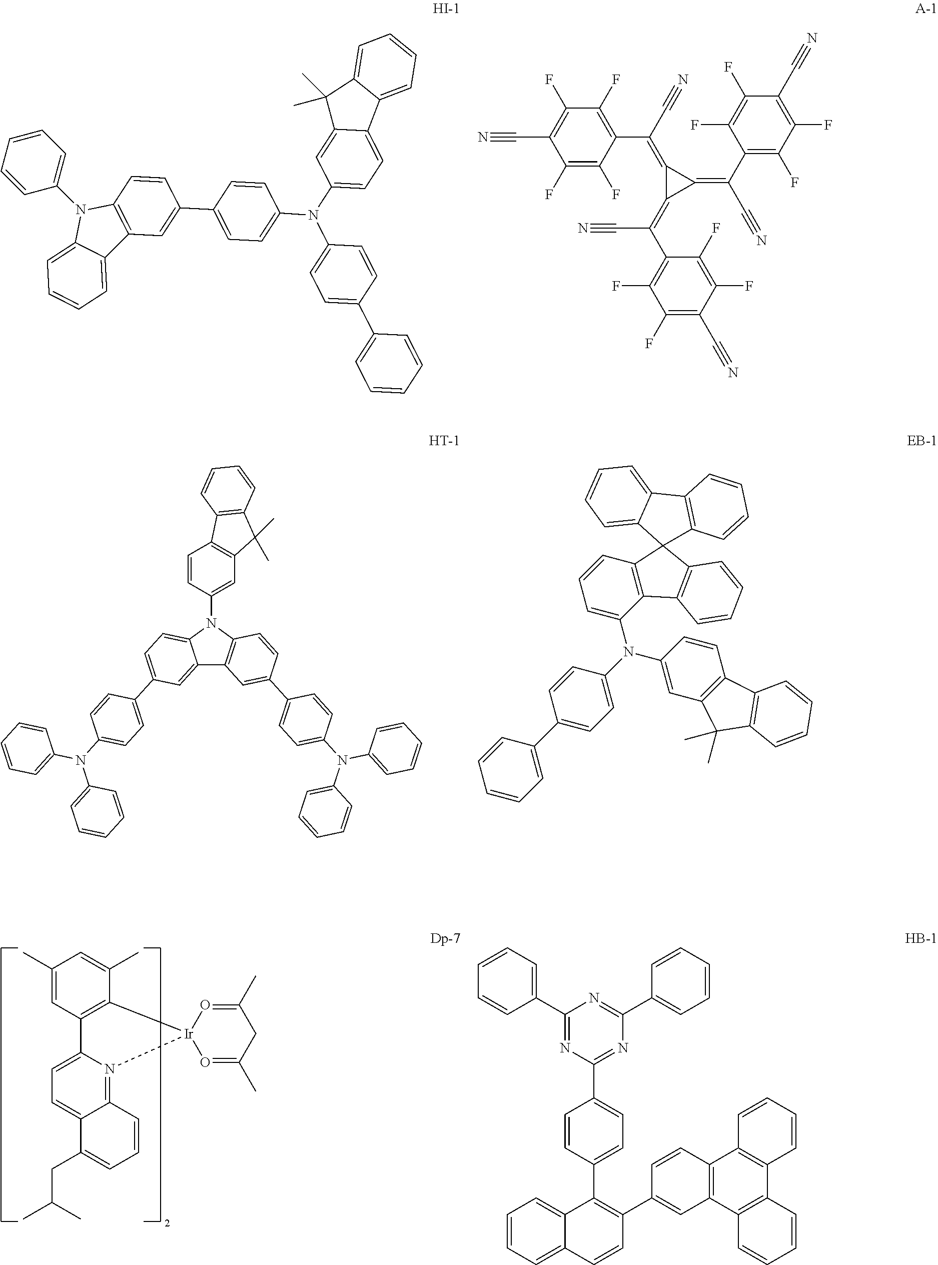

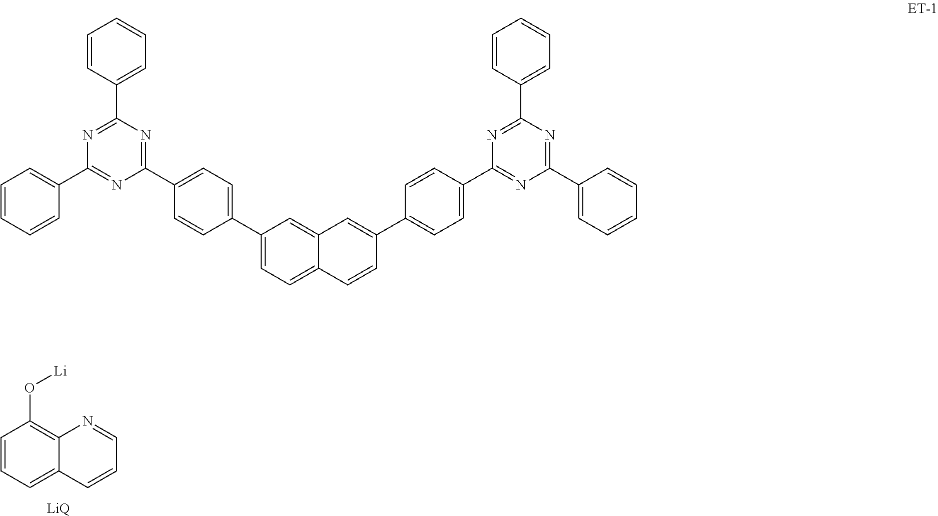

In the above process, the deposition rate of the organic material was maintained at 0.4 to 0.7 Å/sec, the deposition rate of lithium fluoride of the cathode was maintained at 0.3 Å/sec, and the deposition rate of aluminum was maintained at 2 Å/sec. In addition, the degree of vacuum during the deposition was maintained at 2×10⁻⁷ to 5×10⁻⁶ torr, thereby manufacturing an organic light emitting device.

### Examples 1 to 145

Organic light emitting devices were manufactured in the same manner as in Example 1, except that the compounds shown in Table 1 were used instead of Compound 1-2 and Compound 2-1.

### Comparative Examples 1 to 60

Organic light emitting devices were manufactured in the same manner as in Example 1, except that the compounds shown in Table 2 were used instead of Compound 1-2 and Compound 2-1.

### Comparative Examples 61 to 124

Organic light emitting devices were manufactured in the same manner as in Example 1, except that the compounds shown in Table 3 were used instead of Compound 1-2 and Compound 2-1.

The Compounds B-1 to B-12 and C-1 to C-8 used in Comparative Examples 1 to 124 are as follows.

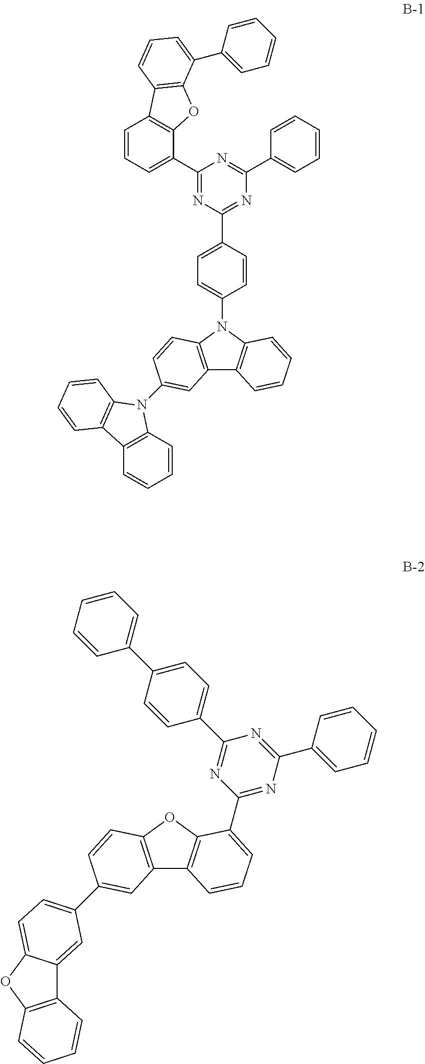 [SMILES: c1ccc(-c2nc(-c3ccc(-n4c5ccccc5c5cc(-n6c7ccccc7c7ccccc76)ccc54)cc3)nc(-c3cccc4c3oc3c(-c5ccccc5)cccc34)n2)cc1 ; c1ccc(-c2ccc(-c3nc(-c4ccccc4)nc(-c4cccc5c4oc4ccc(-c6ccc7oc8ccccc8c7c6)cc45)n3)cc2)cc1]

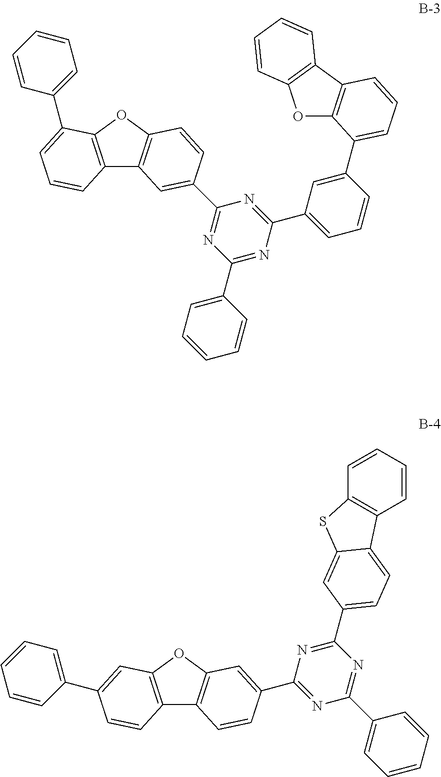 [SMILES: c1ccc(-c2nc(-c3cccc(-c4cccc5c4oc4ccccc45)c3)nc(-c3ccc4oc5c(-c6ccccc6)cccc5c4c3)n2)cc1 ; c1ccc(-c2ccc3c(c2)oc2cc(-c4nc(-c5ccccc5)nc(-c5ccc6c(c5)sc5ccccc56)n4)ccc23)cc1]

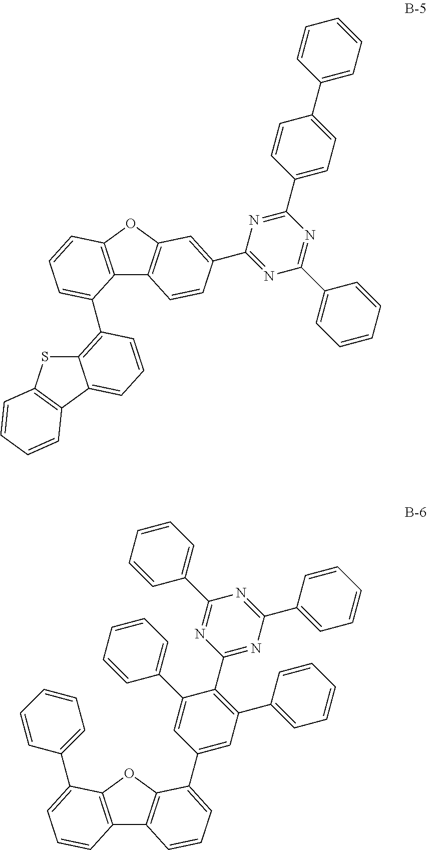 [SMILES: c1ccc(-c2ccc(-c3nc(-c4ccccc4)nc(-c4ccc5c(c4)oc4cccc(-c6cccc7c6sc6ccccc67)c45)n3)cc2)cc1 ; c1ccc(-c2nc(-c3ccccc3)nc(-c3c(-c4ccccc4)cc(-c4cccc5c4oc4c(-c6ccccc6)cccc45)cc3-c3ccccc3)n2)cc1]

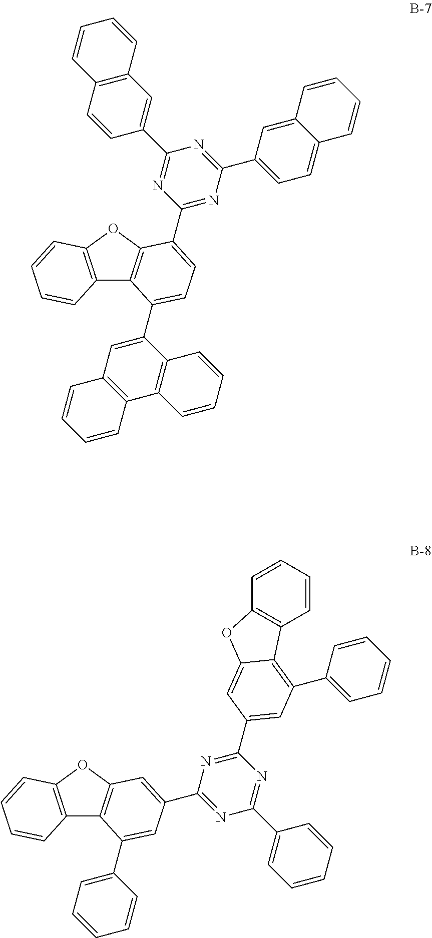 [SMILES: c1ccc2cc(-c3nc(-c4ccc5ccccc5c4)nc(-c4ccc(-c5cc6ccccc6c6ccccc56)c5c4oc4ccccc45)n3)ccc2c1 ; c1ccc(-c2nc(-c3cc(-c4ccccc4)c4c(c3)oc3ccccc34)nc(-c3cc(-c4ccccc4)c4c(c3)oc3ccccc34)n2)cc1]

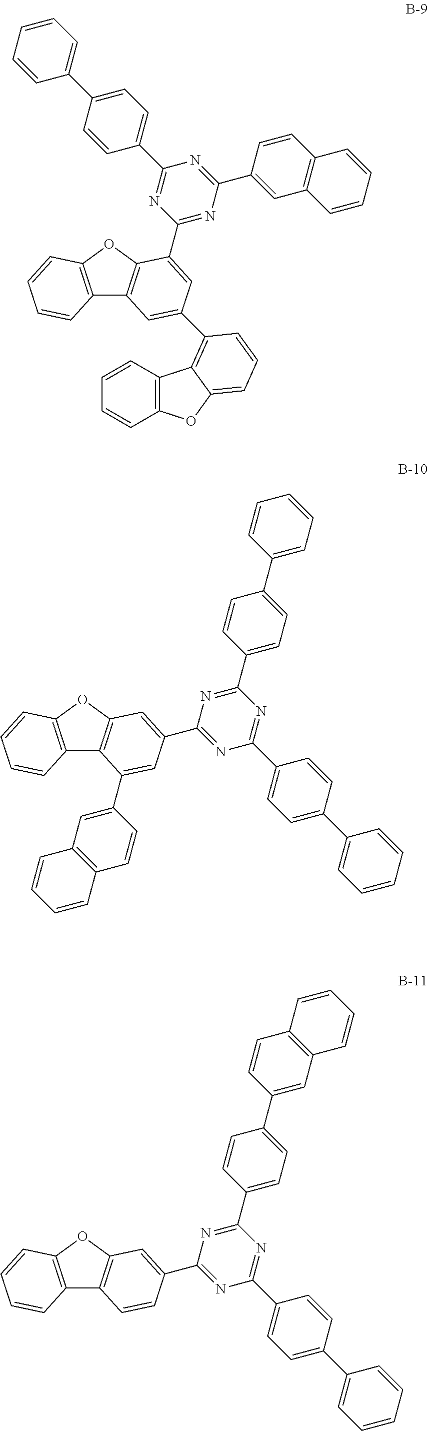 [SMILES: c1ccc(-c2ccc(-c3nc(-c4ccc5ccccc5c4)nc(-c4cc(-c5cccc6oc7ccccc7c56)cc5c4oc4ccccc45)n3)cc2)cc1 ; c1ccc(-c2ccc(-c3nc(-c4ccc(-c5ccccc5)cc4)nc(-c4cc(-c5ccc6ccccc6c5)c5c(c4)oc4ccccc45)n3)cc2)cc1 ; c1ccc(-c2ccc(-c3nc(-c4ccc(-c5ccc6ccccc6c5)cc4)nc(-c4ccc5c(c4)oc4ccccc45)n3)cc2)cc1]

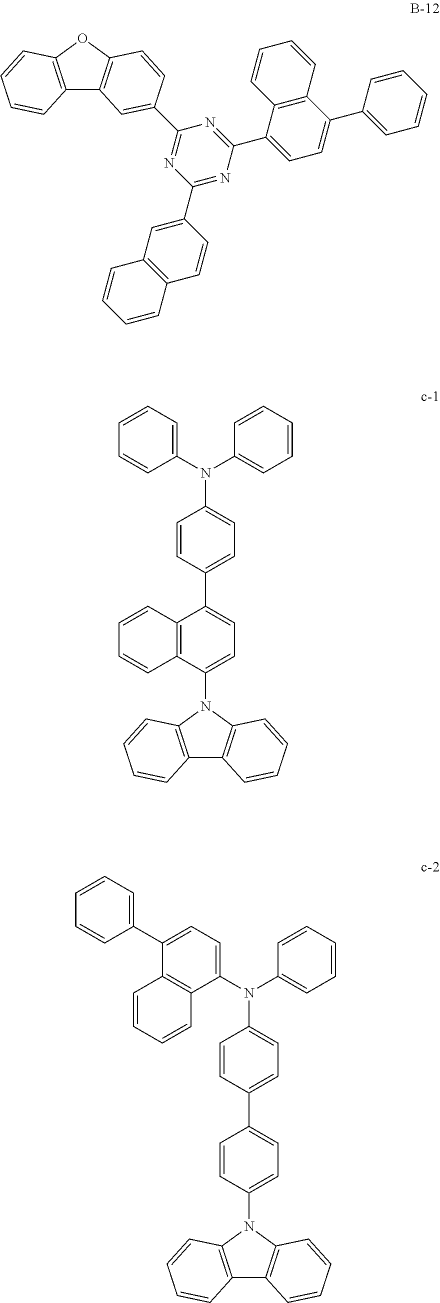 [SMILES: c1ccc(-c2ccc(-c3nc(-c4ccc5ccccc5c4)nc(-c4ccc5oc6ccccc6c5c4)n3)c3ccccc23)cc1 ; c1ccc(N(c2ccccc2)c2ccc(-c3ccc(-n4c5ccccc5c5ccccc54)c4ccccc34)cc2)cc1 ; c1ccc(-c2ccc(N(c3ccccc3)c3ccc(-c4ccc(-n5c6ccccc6c6ccccc65)cc4)cc3)c3ccccc23)cc1]

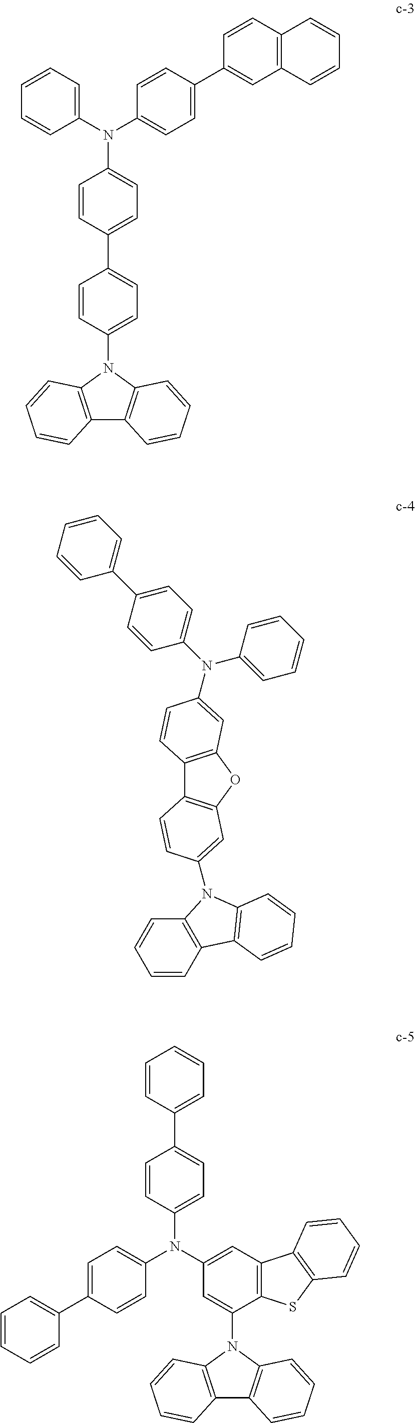 [SMILES: c1ccc(N(c2ccc(-c3ccc(-n4c5ccccc5c5ccccc54)cc3)cc2)c2ccc(-c3ccc4ccccc4c3)cc2)cc1 ; c1ccc(-c2ccc(N(c3ccccc3)c3ccc4c(c3)oc3cc(-n5c6ccccc6c6ccccc65)ccc34)cc2)cc1 ; c1ccc(-c2ccc(N(c3ccc(-c4ccccc4)cc3)c3cc(-n4c5ccccc5c5ccccc54)c4sc5ccccc5c4c3)cc2)cc1]

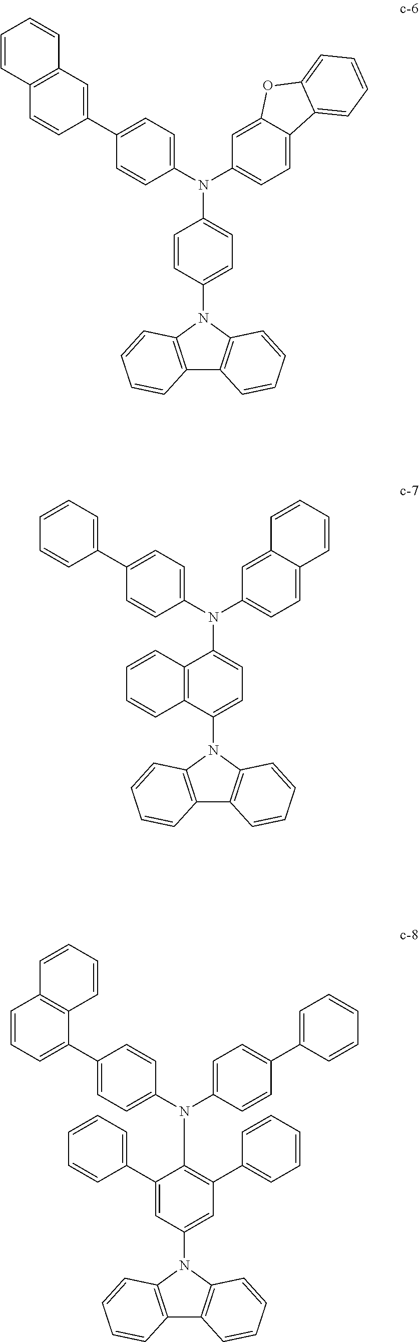 [SMILES: c1ccc2cc(-c3ccc(N(c4ccc(-n5c6ccccc6c6ccccc65)cc4)c4ccc5c(c4)oc4ccccc45)cc3)ccc2c1 ; c1ccc(-c2ccc(N(c3ccc4ccccc4c3)c3ccc(-n4c5ccccc5c5ccccc54)c4ccccc34)cc2)cc1 ; c1ccc(-c2ccc(N(c3ccc(-c4cccc5ccccc45)cc3)c3c(-c4ccccc4)cc(-n4c5ccccc5c5ccccc54)cc3-c3ccccc3)cc2)cc1]

### Experimental Examples

The driving voltage, efficiency, and lifespan were measured by applying a current of 15 mA/cm² to the organic light emitting devices prepared in the above Examples 1 to 145 and Comparative Examples 1 to 124. The lifespan T95 means the time (hr) taken until the initial luminance (6000 nit) decreases to 95%.

The results are shown in Tables 1 to 3 below.

TABLE 1

| | | | | | | Light |
|---|---|---|---|---|---|---|
| | | | Driving | | Lifespan | emitting |
| Sortation | 1st host | 2nd host | voltage(V) | Efficiency(cd/A) | T95(hr) | color |
| Example 1 | compound | compound | 3.83 | 20.00 | 187 | Red |
| | 1-2 | 2-1 | | | | |
| Example 2 | compound | compound | 3.79 | 20.89 | 182 | Red |
| | 1-2 | 2-7 | | | | |
| Example 3 | compound | compound | 3.83 | 23.00 | 184 | Red |
| | 1-2 | 2-13 | | | | |
| Example 4 | compound | compound | 3.79 | 21.76 | 177 | Red |
| | 1-2 | 2-19 | | | | |
| Example 5 | compound | compound | 3.67 | 22.82 | 173 | Red |
| | 1-2 | 2-30 | | | | |
| Example 6 | compound | compound | 3.78 | 22.73 | 183 | Red |
| | 1-3 | 2-2 | | | | |
| Example 7 | compound | compound | 3.67 | 22.44 | 174 | Red |
| | 1-3 | 2-8 | | | | |
| Example 8 | compound | compound | 3.76 | 21.28 | 172 | Red |
| | 1-3 | 2-14 | | | | |
| Example 9 | compound | compound | 3.78 | 19.84 | 181 | Red |
| | 1-3 | 2-20 | | | | |
| Example 10 | compound | compound | 3.75 | 22.68 | 183 | Red |
| | 1-3 | 2-31 | | | | |
| Example 11 | compound | compound | 4.00 | 23.65 | 195 | Red |
| | 1-7 | 2-3 | | | | |
| Example 12 | compound | compound | 3.97 | 21.77 | 201 | Red |
| | 1-7 | 2-9 | | | | |
| Example 13 | compound | compound | 4.00 | 23.68 | 203 | Red |
| | 1-7 | 2-15 | | | | |
| Example 14 | compound | compound | 3.92 | 21.71 | 186 | Red |
| | 1-7 | 2-22 | | | | |
| Example 15 | compound | compound | 4.06 | 21.36 | 201 | Red |
| | 1-7 | 2-32 | | | | |
| Example 16 | compound | compound | 3.90 | 22.99 | 183 | Red |
| | 1-9 | 2-4 | | | | |
| Example 17 | compound | compound | 4.02 | 23.32 | 189 | Red |
| | 1-9 | 2-10 | | | | |
| Example 18 | compound | compound | 4.02 | 21.95 | 196 | Red |
| | 1-9 | 2-16 | | | | |
| Example 19 | compound | compound | 4.03 | 22.96 | 198 | Red |
| | 1-9 | 2-23 | | | | |
| Example 20 | compound | compound | 3.91 | 22.93 | 203 | Red |
| | 1-9 | 2-33 | | | | |
| Example 21 | compound | compound | 3.80 | 22.58 | 226 | Red |
| | 1-11 | 2-5 | | | | |
| Example 22 | compound | compound | 3.75 | 22.95 | 231 | Red |
| | 1-11 | 2-11 | | | | |
| Example 23 | compound | compound | 3.91 | 22.30 | 232 | Red |
| | 1-11 | 2-17 | | | | |
| Example 24 | compound | compound | 3.74 | 22.84 | 217 | Red |
| | 1-11 | 2-24 | | | | |
| Example 25 | compound | compound | 3.78 | 23.01 | 242 | Red |
| | 1-11 | 2-34 | | | | |
| Example 26 | compound | compound | 3.77 | 22.87 | 219 | Red |
| | 1-14 | 2-6 | | | | |
| Example 27 | compound | compound | 3.71 | 22.07 | 222 | Red |
| | 1-14 | 2-12 | | | | |
| Example 28 | compound | compound | 3.75 | 21.05 | 229 | Red |
| | 1-14 | 2-18 | | | | |
| Example 29 | compound | compound | 3.86 | 21.91 | 221 | Red |
| | 1-14 | 2-26 | | | | |
| Example 30 | compound | compound | 3.76 | 22.75 | 227 | Red |
| | 1-14 | 2-35 | | | | |
| Example 31 | compound | compound | 3.89 | 23.20 | 219 | Red |
| | 1-15 | 2-1 | | | | |
| Example 32 | compound | compound | 3.83 | 22.95 | 210 | Red |
| | 1-15 | 2-7 | | | | |
| Example 33 | compound | compound | 3.84 | 22.30 | 221 | Red |
| | 1-15 | 2-13 | | | | |
| Example 34 | compound | compound | 3.79 | 22.84 | 201 | Red |
| | 1-15 | 2-19 | | | | |
| Example 35 | compound | compound | 3.95 | 23.01 | 216 | Red |
| | 1-15 | 2-30 | | | | |
| Example 36 | compound | compound | 3.93 | 22.87 | 208 | Red |
| | 1-16 | 2-2 | | | | |
| Example 37 | compound | compound | 3.81 | 22.07 | 195 | Red |
| | 1-16 | 2-8 | | | | |
| Example 38 | compound | compound | 3.83 | 21.05 | 196 | Red |
| | 1-16 | 2-14 | | | | |
| Example 39 | compound | compound | 3.96 | 21.91 | 208 | Red |
| | 1-16 | 2-20 | | | | |
| Example 40 | compound | compound | 3.93 | 22.75 | 199 | Red |
| | 1-16 | 2-31 | | | | |
| Example 41 | compound | compound | 3.67 | 22.67 | 179 | Red |
| | 1-17 | 2-3 | | | | |
| Example 42 | compound | compound | 3.71 | 19.55 | 189 | Red |
| | 1-17 | 2-9 | | | | |
| Example 43 | compound | compound | 3.82 | 19.79 | 187 | Red |
| | 1-17 | 2-15 | | | | |
| Example 44 | compound | compound | 3.71 | 22.58 | 172 | Red |
| | 1-17 | 2-22 | | | | |
| Example 45 | compound | compound | 3.72 | 20.91 | 179 | Red |
| | 1-17 | 2-32 | | | | |
| Example 46 | compound | compound | 3.78 | 20.60 | 173 | Red |
| | 1-20 | 2-4 | | | | |
| Example 47 | compound | compound | 3.77 | 19.65 | 179 | Red |
| | 1-20 | 2-10 | | | | |
| Example 48 | compound | compound | 3.74 | 20.94 | 177 | Red |
| | 1-20 | 2-16 | | | | |
| Example 49 | compound | compound | 3.69 | 19.43 | 179 | Red |
| | 1-20 | 2-23 | | | | |
| Example 50 | compound | compound | 3.84 | 20.91 | 178 | Red |
| | 1-20 | 2-33 | | | | |
| Example 51 | compound | compound | 3.69 | 19.36 | 180 | Red |
| | 1-22 | 2-5 | | | | |
| Example 52 | compound | compound | 3.52 | 18.24 | 163 | Red |
| | 1-22 | 2-11 | | | | |
| Example 53 | compound | compound | 3.50 | 19.26 | 166 | Red |
| | 1-22 | 2-17 | | | | |
| Example 54 | compound | compound | 3.71 | 18.15 | 164 | Red |
| | 1-22 | 2-24 | | | | |
| Example 55 | compound | compound | 3.64 | 18.91 | 186 | Red |
| | 1-22 | 2-34 | | | | |
| Example 56 | compound | compound | 3.71 | 19.08 | 165 | Red |
| | 1-24 | 2-6 | | | | |
| Example 57 | compound | compound | 3.72 | 18.90 | 188 | Red |
| | 1-24 | 2-12 | | | | |
| Example 58 | compound | compound | 3.65 | 18.99 | 183 | Red |
| | 1-24 | 2-18 | | | | |
| Example 59 | compound | compound | 3.54 | 19.23 | 182 | Red |
| | 1-24 | 2-26 | | | | |
| Example 60 | compound | compound | 3.73 | 18.65 | 174 | Red |
| | 1-24 | 2-35 | | | | |
| Example 61 | compound | compound | 3.84 | 22.42 | 246 | Red |
| | 1-27 | 2-1 | | | | |
| Example 62 | compound | compound | 3.79 | 23.81 | 246 | Red |
| | 1-27 | 2-7 | | | | |
| Example 63 | compound | compound | 3.82 | 22.40 | 231 | Red |
| | 1-27 | 2-13 | | | | |
| Example 64 | compound | compound | 3.74 | 22.82 | 244 | Red |
| | 1-27 | 2-19 | | | | |
| Example 65 | compound | compound | 3.87 | 21.29 | 227 | Red |
| | 1-27 | 2-30 | | | | |
| Example 66 | compound | compound | 3.77 | 22.37 | 246 | Red |
| | 1-28 | 2-2 | | | | |
| Example 67 | compound | compound | 3.79 | 23.68 | 246 | Red |
| | 1-28 | 2-8 | | | | |
| Example 68 | compound | compound | 3.88 | 22.92 | 237 | Red |
| | 1-28 | 2-14 | | | | |
| Example 69 | compound | compound | 3.76 | 23.47 | 241 | Red |
| | 1-28 | 2-20 | | | | |
| Example 70 | compound | compound | 3.91 | 23.95 | 224 | Red |
| | 1-28 | 2-31 | | | | |
| Example 71 | compound | compound | 3.69 | 19.31 | 177 | Red |
| | 1-31 | 2-3 | | | | |
| Example 72 | compound | compound | 3.71 | 20.95 | 192 | Red |
| | 1-31 | 2-9 | | | | |
| Example 73 | compound | compound | 3.80 | 19.59 | 190 | Red |
| | 1-31 | 2-15 | | | | |
| Example 74 | compound | compound | 3.80 | 22.99 | 173 | Red |
| | 1-31 | 2-22 | | | | |
| Example 75 | compound | compound | 3.69 | 20.94 | 187 | Red |
| | 1-31 | 2-32 | | | | |
| Example 76 | compound | compound | 3.76 | 21.81 | 182 | Red |
| | 1-33 | 2-4 | | | | |
| Example 77 | compound | compound | 3.72 | 22.16 | 185 | Red |
| | 1-33 | 2-10 | | | | |
| Example 78 | compound | compound | 3.79 | 22.93 | 173 | Red |
| | 1-33 | 2-16 | | | | |
| Example 79 | compound | compound | 3.76 | 21.10 | 180 | Red |
| | 1-33 | 2-23 | | | | |
| Example 80 | compound | compound | 3.79 | 19.42 | 194 | Red |
| | 1-33 | 2-33 | | | | |
| Example 81 | compound | compound | 3.83 | 22.51 | 227 | Red |
| | 1-37 | 2-5 | | | | |
| Example 82 | compound | compound | 3.80 | 23.54 | 224 | Red |
| | 1-37 | 2-11 | | | | |
| Example 83 | compound | compound | 3.93 | 23.79 | 216 | Red |
| | 1-37 | 2-17 | | | | |
| Example 84 | compound | compound | 3.74 | 23.50 | 226 | Red |
| | 1-37 | 2-24 | | | | |
| Example 85 | compound | compound | 3.91 | 23.55 | 215 | Red |
| | 1-37 | 2-34 | | | | |
| Example 86 | compound | compound | 3.86 | 21.38 | 214 | Red |
| | 1-38 | 2-6 | | | | |
| Example 87 | compound | compound | 3.80 | 21.77 | 227 | Red |
| | 1-38 | 2-12 | | | | |
| Example 88 | compound | compound | 3.80 | 22.40 | 228 | Red |
| | 1-38 | 2-18 | | | | |
| Example 89 | compound | compound | 3.72 | 22.15 | 227 | Red |
| | 1-38 | 2-26 | | | | |
| Example 90 | compound | compound | 3.73 | 22.48 | 224 | Red |
| | 1-38 | 2-35 | | | | |
| Example 91 | compound | compound | 3.87 | 22.51 | 209 | Red |
| | 1-40 | 2-1 | | | | |
| Example 92 | compound | compound | 3.97 | 21.86 | 216 | Red |
| | 1-40 | 2-7 | | | | |
| Example 93 | compound | compound | 3.91 | 21.72 | 211 | Red |
| | 1-40 | 2-13 | | | | |
| Example 94 | compound | compound | 3.97 | 21.39 | 205 | Red |
| | 1-40 | 2-19 | | | | |
| Example 95 | compound | compound | 3.82 | 22.80 | 200 | Red |
| | 1-40 | 2-30 | | | | |
| Example 96 | compound | compound | 3.97 | 23.56 | 208 | Red |
| | 1-41 | 2-2 | | | | |
| Example 97 | compound | compound | 3.86 | 21.66 | 201 | Red |
| | 1-41 | 2-8 | | | | |
| Example 98 | compound | compound | 3.96 | 22.46 | 214 | Red |
| | 1-41 | 2-14 | | | | |
| Example 99 | compound | compound | 3.80 | 23.85 | 219 | Red |
| | 1-41 | 2-20 | | | | |
| Example 100 | compound | compound | 3.95 | 23.45 | 217 | Red |
| | 1-41 | 2-31 | | | | |
| Example 101 | compound | compound | 3.50 | 18.75 | 163 | Red |
| | 1-43 | 2-3 | | | | |
| Example 102 | compound | compound | 3.61 | 18.44 | 185 | Red |
| | 1-43 | 2-9 | | | | |
| Example 103 | compound | compound | 3.62 | 18.93 | 171 | Red |
| | 1-43 | 2-15 | | | | |
| Example 104 | compound | compound | 3.70 | 18.98 | 172 | Red |
| | 1-43 | 2-22 | | | | |
| Example 105 | compound | compound | 3.63 | 18.30 | 176 | Red |
| | 1-43 | 2-32 | | | | |
| Example 106 | compound | compound | 3.72 | 18.56 | 175 | Red |
| | 1-45 | 2-4 | | | | |
| Example 107 | compound | compound | 3.73 | 19.05 | 168 | Red |
| | 1-45 | 2-10 | | | | |
| Example 108 | compound | compound | 3.67 | 18.28 | 169 | Red |
| | 1-45 | 2-16 | | | | |
| Example 109 | compound | compound | 3.62 | 18.50 | 179 | Red |
| | 1-45 | 2-23 | | | | |
| Example 110 | compound | compound | 3.55 | 18.99 | 166 | Red |
| | 1-45 | 2-33 | | | | |
| Example 101 | compound | compound | 3.61 | 18.63 | 165 | Red |
| | 1-47 | 2-5 | | | | |
| Example 102 | compound | compound | 3.50 | 18.21 | 168 | Red |
| | 1-47 | 2-11 | | | | |
| Example 103 | compound | compound | 3.56 | 18.92 | 177 | Red |
| | 1-47 | 2-17 | | | | |
| Example 104 | compound | compound | 3.64 | 19.15 | 163 | Red |
| | 1-47 | 2-24 | | | | |
| Example 105 | compound | compound | 3.67 | 18.96 | 175 | Red |
| | 1-47 | 2-34 | | | | |
| Example 106 | compound | compound | 3.64 | 18.78 | 186 | Red |
| | 1-48 | 2-6 | | | | |
| Example 107 | compound | compound | 3.68 | 18.99 | 181 | Red |
| | 1-48 | 2-12 | | | | |
| Example 108 | compound | compound | 3.67 | 18.34 | 179 | Red |
| | 1-48 | 2-18 | | | | |
| Example 109 | compound | compound | 3.54 | 19.10 | 165 | Red |
| | 1-48 | 2-26 | | | | |
| Example 110 | compound | compound | 3.59 | 18.57 | 185 | Red |
| | 1-48 | 2-35 | | | | |
| Example 111 | compound | compound | 3.78 | 21.07 | 219 | Red |
| | 1-52 | 2-1 | | | | |
| Example 112 | compound | compound | 3.73 | 23.77 | 229 | Red |
| | 1-52 | 2-7 | | | | |
| Example 113 | compound | compound | 3.79 | 23.29 | 232 | Red |
| | 1-52 | 2-13 | | | | |
| Example 114 | compound | compound | 3.80 | 22.81 | 215 | Red |
| | 1-52 | 2-19 | | | | |
| Example 115 | compound | compound | 3.91 | 21.16 | 217 | Red |
| | 1-52 | 2-30 | | | | |
| Example 116 | compound | compound | 3.99 | 22.69 | 204 | Red |
| | 1-53 | 2-2 | | | | |
| Example 117 | compound | compound | 3.88 | 22.82 | 184 | Red |
| | 1-53 | 2-8 | | | | |
| Example 118 | compound | compound | 3.80 | 24.00 | 194 | Red |
| | 1-53 | 2-14 | | | | |
| Example 119 | compound | compound | 3.84 | 23.52 | 186 | Red |
| | 1-53 | 2-20 | | | | |
| Example 120 | compound | compound | 3.91 | 22.08 | 191 | Red |
| | 1-53 | 2-31 | | | | |
| Example 121 | compound | compound | 3.75 | 21.35 | 195 | Red |
| | 1-55 | 2-3 | | | | |
| Example 122 | compound | compound | 3.88 | 23.46 | 183 | Red |
| | 1-55 | 2-9 | | | | |
| Example 123 | compound | compound | 4.07 | 21.69 | 189 | Red |
| | 1-55 | 2-15 | | | | |
| Example 124 | compound | compound | 3.94 | 22.89 | 189 | Red |
| | 1-55 | 2-22 | | | | |
| Example 125 | compound | compound | 3.75 | 22.68 | 194 | Red |
| | 1-55 | 2-32 | | | | |
| Example 126 | compound | compound | 3.93 | 23.73 | 232 | Red |
| | 1-56 | 2-4 | | | | |
| Example 127 | compound | compound | 3.88 | 21.22 | 232 | Red |
| | 1-56 | 2-10 | | | | |
| Example 128 | compound | compound | 3.84 | 21.96 | 213 | Red |
| | 1-56 | 2-16 | | | | |
| Example 129 | compound | compound | 3.71 | 22.84 | 230 | Red |
| | 1-56 | 2-23 | | | | |
| Example 130 | compound | compound | 3.74 | 23.94 | 226 | Red |
| | 1-56 | 2-33 | | | | |
| Example 131 | compound | compound | 3.91 | 21.38 | 239 | Red |
| | 1-57 | 2-5 | | | | |
| Example 132 | compound | compound | 3.77 | 22.97 | 228 | Red |
| | 1-57 | 2-11 | | | | |
| Example 133 | compound | compound | 3.89 | 21.05 | 223 | Red |
| | 1-57 | 2-17 | | | | |
| Example 134 | compound | compound | 3.85 | 22.02 | 242 | Red |
| | 1-57 | 2-24 | | | | |
| Example 135 | compound | compound | 3.81 | 23.13 | 234 | Red |
| | 1-57 | 2-34 | | | | |
| Example 136 | compound | compound | 3.94 | 22.63 | 184 | Red |
| | 1-58 | 2-6 | | | | |
| Example 137 | compound | compound | 3.92 | 22.70 | 185 | Red |
| | 1-58 | 2-12 | | | | |
| Example 138 | compound | compound | 3.78 | 21.68 | 185 | Red |
| | 1-58 | 2-18 | | | | |
| Example 139 | compound | compound | 4.01 | 22.91 | 192 | Red |
| | 1-58 | 2-26 | | | | |
| Example 140 | compound | compound | 3.92 | 21.66 | 192 | Red |
| | 1-58 | 2-35 | | | | |
| Example 141 | compound | compound | 3.80 | 23.69 | 191 | Red |
| | 1-60 | 2-1 | | | | |
| Example 142 | compound | compound | 3.85 | 23.47 | 182 | Red |
| | 1-60 | 2-7 | | | | |
| Example 143 | compound | compound | 3.82 | 22.61 | 192 | Red |
| | 1-60 | 2-13 | | | | |
| Example 144 | compound | compound | 3.98 | 21.07 | 190 | Red |
| | 1-60 | 2-19 | | | | |
| Example 145 | compound | compound | 3.99 | 21.15 | 190 | Red |
| | 1-60 | 2-30 | | | | |

TABLE 2

| | | | | | | Light |
|---|---|---|---|---|---|---|
| | | | Driving | | Lifespan | emitting |
| Sortation | 1st host | 2nd host | voltage(V) | Efficiency(cd/A) | T95(hr) | color |
| Comparative | compound | compound | 4.20 | 15.07 | 120 | Red |
| Example 1 | B-1 | 2-1 | | | | |
| Comparative | compound | compound | 4.34 | 14.53 | 142 | Red |
| Example 2 | B-1 | 2-7 | | | | |
| Comparative | compound | compound | 4.30 | 14.76 | 127 | Red |
| Example 3 | B-1 | 2-13 | | | | |
| Comparative | compound | compound | 4.24 | 14.72 | 145 | Red |
| Example 4 | B-1 | 2-19 | | | | |
| Comparative | compound | compound | 4.15 | 15.00 | 122 | Red |
| Example 5 | B-1 | 2-30 | | | | |
| Comparative | compound | compound | 4.17 | 14.96 | 128 | Red |
| Example 6 | B-2 | 2-2 | | | | |
| Comparative | compound | compound | 4.24 | 15.03 | 135 | Red |
| Example 7 | B-2 | 2-8 | | | | |
| Comparative | compound | compound | 4.26 | 14.21 | 141 | Red |
| Example 8 | B-2 | 2-14 | | | | |
| Comparative | compound | compound | 4.31 | 14.94 | 140 | Red |
| Example 9 | B-2 | 2-20 | | | | |
| Comparative | compound | compound | 4.13 | 14.50 | 123 | Red |
| Example 10 | B-2 | 2-31 | | | | |
| Comparative | compound | compound | 4.28 | 16.31 | 107 | Red |
| Example 11 | B-3 | 2-3 | | | | |
| Comparative | compound | compound | 4.30 | 15.71 | 102 | Red |
| Example 12 | B-3 | 2-9 | | | | |
| Comparative | compound | compound | 4.26 | 15.73 | 107 | Red |
| Example 13 | B-3 | 2-15 | | | | |
| Comparative | compound | compound | 4.30 | 15.77 | 123 | Red |
| Example 14 | B-3 | 2-22 | | | | |
| Comparative | compound | compound | 4.17 | 16.22 | 105 | Red |
| Example 15 | B-3 | 2-32 | | | | |
| Comparative | compound | compound | 4.19 | 16.05 | 121 | Red |
| Example 16 | B-4 | 2-4 | | | | |
| Comparative | compound | compound | 4.25 | 15.53 | 104 | Red |
| Example 17 | B-4 | 2-10 | | | | |
| Comparative | compound | compound | 4.17 | 16.47 | 104 | Red |
| Example 18 | B-4 | 2-16 | | | | |
| Comparative | compound | compound | 4.20 | 15.81 | 113 | Red |
| Example 19 | B-4 | 2-23 | | | | |
| Comparative | compound | compound | 4.20 | 16.02 | 119 | Red |
| Example 20 | B-4 | 2-33 | | | | |
| Comparative | compound | compound | 4.12 | 14.68 | 125 | Red |
| Example 21 | B-5 | 2-5 | | | | |
| Comparative | compound | compound | 4.26 | 15.01 | 137 | Red |
| Example 22 | B-5 | 2-11 | | | | |
| Comparative | compound | compound | 4.18 | 14.52 | 138 | Red |
| Example 23 | B-5 | 2-17 | | | | |
| Comparative | compound | compound | 4.29 | 14.71 | 140 | Red |
| Example 24 | B-5 | 2-24 | | | | |
| Comparative | compound | compound | 4.16 | 14.83 | 123 | Red |
| Example 25 | B-5 | 2-34 | | | | |
| Comparative | compound | compound | 4.18 | 14.75 | 141 | Red |
| Example 26 | B-6 | 2-6 | | | | |
| Comparative | compound | compound | 4.33 | 14.74 | 141 | Red |
| Example 27 | B-6 | 2-12 | | | | |
| Comparative | compound | compound | 4.15 | 14.35 | 123 | Red |
| Example 28 | B-6 | 2-18 | | | | |
| Comparative | compound | compound | 4.19 | 15.03 | 136 | Red |
| Example 29 | B-6 | 2-26 | | | | |
| Comparative | compound | compound | 4.12 | 14.56 | 139 | Red |
| Example 30 | B-6 | 2-35 | | | | |
| Comparative | compound | compound | 4.29 | 16.43 | 120 | Red |
| Example 31 | B-7 | 2-1 | | | | |
| Comparative | compound | compound | 4.25 | 15.96 | 114 | Red |
| Example 32 | B-7 | 2-7 | | | | |
| Comparative | compound | compound | 4.22 | 15.38 | 115 | Red |
| Example 33 | B-7 | 2-13 | | | | |
| Comparative | compound | compound | 4.23 | 16.65 | 114 | Red |
| Example 34 | B-7 | 2-19 | | | | |
| Comparative | compound | compound | 4.36 | 16.15 | 112 | Red |
| Example 35 | B-7 | 2-30 | | | | |
| Comparative | compound | compound | 4.30 | 15.74 | 105 | Red |
| Example 36 | B-8 | 2-2 | | | | |
| Comparative | compound | compound | 4.18 | 16.54 | 116 | Red |
| Example 37 | B-8 | 2-8 | | | | |
| Comparative | compound | compound | 4.27 | 16.69 | 113 | Red |
| Example 38 | B-8 | 2-14 | | | | |
| Comparative | compound | compound | 4.17 | 15.75 | 105 | Red |
| Example 39 | B-8 | 2-20 | | | | |
| Comparative | compound | compound | 4.21 | 16.41 | 120 | Red |
| Example 40 | B-8 | 2-31 | | | | |
| Comparative | compound | compound | 4.28 | 14.50 | 145 | Red |
| Example 41 | B-9 | 2-3 | | | | |
| Comparative | compound | compound | 4.26 | 14.69 | 123 | Red |
| Example 42 | B-9 | 2-9 | | | | |
| Comparative | compound | compound | 4.32 | 15.08 | 131 | Red |
| Example 43 | B-9 | 2-15 | | | | |
| Comparative | compound | compound | 4.25 | 14.98 | 123 | Red |
| Example 44 | B-9 | 2-22 | | | | |
| Comparative | compound | compound | 4.28 | 14.45 | 126 | Red |
| Example 45 | B-9 | 2-32 | | | | |
| Comparative | compound | compound | 4.15 | 14.69 | 140 | Red |
| Example 46 | B-10 | 2-4 | | | | |
| Comparative | compound | compound | 4.28 | 14.33 | 148 | Red |
| Example 47 | B-10 | 2-10 | | | | |
| Comparative | compound | compound | 4.25 | 14.53 | 135 | Red |
| Example 48 | B-10 | 2-16 | | | | |
| Comparative | compound | compound | 4.11 | 14.66 | 125 | Red |
| Example 49 | B-10 | 2-23 | | | | |
| Comparative | compound | compound | 4.31 | 14.69 | 124 | Red |
| Example 50 | B-10 | 2-33 | | | | |
| Comparative | compound | compound | 4.32 | 15.34 | 113 | Red |
| Example 51 | B-11 | 2-5 | | | | |
| Comparative | compound | compound | 4.36 | 15.56 | 111 | Red |
| Example 52 | B-11 | 2-11 | | | | |
| Comparative | compound | compound | 4.23 | 15.76 | 110 | Red |
| Example 53 | B-11 | 2-17 | | | | |
| Comparative | compound | compound | 4.27 | 16.62 | 105 | Red |
| Example 54 | B-11 | 2-24 | | | | |
| Comparative | compound | compound | 4.32 | 16.13 | 104 | Red |
| Example 55 | B-11 | 2-34 | | | | |
| Comparative | compound | compound | 4.25 | 15.48 | 112 | Red |
| Example 56 | B-12 | 2-6 | | | | |
| Comparative | compound | compound | 4.23 | 16.34 | 120 | Red |
| Example 57 | B-12 | 2-12 | | | | |
| Comparative | compound | compound | 4.18 | 16.60 | 110 | Red |
| Example 58 | B-12 | 2-18 | | | | |
| Comparative | compound | compound | 4.30 | 16.45 | 119 | Red |
| Example 59 | B-12 | 2-26 | | | | |
| Comparative | compound | compound | 4.21 | 15.23 | 122 | Red |
| Example 60 | B-12 | 2-35 | | | | |

TABLE 3

| | | | | | | Light |
|---|---|---|---|---|---|---|
| | | | Driving | | Lifespan | emitting |
| Sortation | 1st host | 2nd host | voltage(V) | Efficiency(cd/A) | T95(hr) | color |
| Comparative | compound | compound | 4.24 | 14.92 | 141 | Red |
| Example 61 | 1-2 | C-1 | | | | |
| Comparative | compound | compound | 4.18 | 15.12 | 147 | Red |
| Example 62 | 1-11 | C-1 | | | | |
| Comparative | compound | compound | 4.15 | 14.87 | 137 | Red |
| Example 63 | 1-15 | C-1 | | | | |
| Comparative | compound | compound | 4.21 | 14.96 | 121 | Red |
| Example 64 | 1-28 | C-1 | | | | |
| Comparative | compound | compound | 4.18 | 15.33 | 129 | Red |
| Example 65 | 1-33 | C-1 | | | | |
| Comparative | compound | compound | 4.19 | 15.88 | 135 | Red |
| Example 66 | 1-40 | C-1 | | | | |
| Comparative | compound | compound | 4.11 | 15.17 | 133 | Red |
| Example 67 | 1-43 | C-1 | | | | |
| Comparative | compound | compound | 4.08 | 15.19 | 128 | Red |
| Example 68 | 1-55 | C-1 | | | | |
| Comparative | compound | compound | 4.41 | 15.06 | 149 | Red |
| Example 69 | 1-3 | C-2 | | | | |
| Comparative | compound | compound | 4.41 | 14.29 | 144 | Red |
| Example 70 | 1-7 | C-2 | | | | |
| Comparative | compound | compound | 4.45 | 15.09 | 149 | Red |
| Example 71 | 1-17 | C-2 | | | | |
| Comparative | compound | compound | 4.40 | 14.73 | 136 | Red |
| Example 72 | 1-24 | C-2 | | | | |
| Comparative | compound | compound | 4.48 | 14.35 | 142 | Red |
| Example 73 | 1-37 | C-2 | | | | |
| Comparative | compound | compound | 4.30 | 14.52 | 125 | Red |
| Example 74 | 1-47 | C-2 | | | | |
| Comparative | compound | compound | 4.45 | 14.44 | 126 | Red |
| Example 75 | 1-48 | C-2 | | | | |
| Comparative | compound | compound | 4.44 | 14.55 | 126 | Red |
| Example 76 | 1-58 | C-2 | | | | |
| Comparative | compound | compound | 4.11 | 15.76 | 146 | Red |
| Example 77 | 1-9 | C-3 | | | | |
| Comparative | compound | compound | 4.16 | 14.93 | 121 | Red |
| Example 78 | 1-16 | C-3 | | | | |
| Comparative | compound | compound | 4.11 | 15.04 | 121 | Red |
| Example 79 | 1-22 | C-3 | | | | |
| Comparative | compound | compound | 4.08 | 15.82 | 137 | Red |
| Example 80 | 1-38 | C-3 | | | | |
| Comparative | compound | compound | 4.12 | 15.74 | 136 | Red |
| Example 81 | 1-41 | C-3 | | | | |
| Comparative | compound | compound | 4.08 | 15.39 | 134 | Red |
| Example 82 | 1-45 | C-3 | | | | |
| Comparative | compound | compound | 4.19 | 14.88 | 146 | Red |
| Example 83 | 1-53 | C-3 | | | | |
| Comparative | compound | compound | 4.12 | 15.71 | 127 | Red |
| Example 84 | 1-57 | C-3 | | | | |
| Comparative | compound | compound | 4.34 | 14.81 | 142 | Red |
| Example 85 | 1-2 | C-4 | | | | |
| Comparative | compound | compound | 4.38 | 14.68 | 120 | Red |
| Example 86 | 1-14 | C-4 | | | | |
| Comparative | compound | compound | 4.46 | 15.09 | 118 | Red |
| Example 87 | 1-20 | C-4 | | | | |
| Comparative | compound | compound | 4.35 | 14.41 | 126 | Red |
| Example 88 | 1-27 | C-4 | | | | |
| Comparative | compound | compound | 4.50 | 14.80 | 136 | Red |
| Example 89 | 1-31 | C-4 | | | | |
| Comparative | compound | compound | 4.41 | 14.44 | 138 | Red |
| Example 90 | 1-52 | C-4 | | | | |
| Comparative | compound | compound | 4.32 | 14.86 | 124 | Red |
| Example 91 | 1-56 | C-4 | | | | |
| Comparative | compound | compound | 4.32 | 14.51 | 129 | Red |
| Example 92 | 1-60 | C-4 | | | | |
| Comparative | compound | compound | 4.41 | 14.69 | 139 | Red |
| Example 93 | 1-2 | C-5 | | | | |
| Comparative | compound | compound | 4.33 | 14.49 | 132 | Red |
| Example 94 | 1-11 | C-5 | | | | |
| Comparative | compound | compound | 4.33 | 14.31 | 135 | Red |
| Example 95 | 1-15 | C-5 | | | | |
| Comparative | compound | compound | 4.42 | 14.31 | 146 | Red |
| Example 96 | 1-28 | C-5 | | | | |
| Comparative | compound | compound | 4.44 | 14.68 | 117 | Red |
| Example 97 | 1-33 | C-5 | | | | |
| Comparative | compound | compound | 4.41 | 15.09 | 148 | Red |
| Example 98 | 1-40 | C-5 | | | | |
| Comparative | compound | compound | 4.33 | 14.26 | 144 | Red |
| Example 99 | 1-43 | C-5 | | | | |
| Comparative | compound | compound | 4.42 | 14.24 | 142 | Red |
| Example 100 | 1-55 | C-5 | | | | |
| Comparative | compound | compound | 4.34 | 14.69 | 139 | Red |
| Example 101 | 1 -3 | C-6 | | | | |
| Comparative | compound | compound | 4.26 | 14.49 | 132 | Red |
| Example 102 | 1-7 | C-6 | | | | |
| Comparative | compound | compound | 4.21 | 14.31 | 135 | Red |
| Example 103 | 1-17 | C-6 | | | | |
| Comparative | compound | compound | 4.25 | 14.31 | 146 | Red |
| Example 104 | 1-24 | C-6 | | | | |
| Comparative | compound | compound | 4.12 | 14.68 | 117 | Red |
| Example 105 | 1-37 | C-6 | | | | |
| Comparative | compound | compound | 4.24 | 15.09 | 148 | Red |
| Example 106 | 1-47 | C-6 | | | | |
| Comparative | compound | compound | 4.22 | 14.26 | 144 | Red |
| Example 107 | 1-48 | C-6 | | | | |
| Comparative | compound | compound | 4.25 | 14.24 | 142 | Red |
| Example 108 | 1-58 | C-6 | | | | |
| Comparative | compound | compound | 4.29 | 15.49 | 120 | Red |
| Example 109 | 1-9 | C-7 | | | | |
| Comparative | compound | compound | 4.23 | 16.20 | 123 | Red |
| Example 110 | 1-16 | C-7 | | | | |
| Comparative | compound | compound | 4.29 | 15.52 | 107 | Red |
| Example 111 | 1-22 | C-7 | | | | |
| Comparative | compound | compound | 4.21 | 15.45 | 123 | Red |
| Example 112 | 1-38 | C-7 | | | | |
| Comparative | compound | compound | 4.26 | 16.21 | 105 | Red |
| Example 113 | 1-41 | C-7 | | | | |
| Comparative | compound | compound | 4.28 | 15.23 | 102 | Red |
| Example 114 | 1-45 | C-7 | | | | |
| Comparative | compound | compound | 4.23 | 15.44 | 104 | Red |
| Example 115 | 1-53 | C-7 | | | | |
| Comparative | compound | compound | 4.31 | 16.49 | 117 | Red |
| Example 116 | 1-57 | C-7 | | | | |
| Comparative | compound | compound | 4.25 | 15.27 | 102 | Red |
| Example 117 | 1-2 | C-8 | | | | |
| Comparative | compound | compound | 4.26 | 15.38 | 103 | Red |
| Example 118 | 1-14 | C-8 | | | | |
| Comparative | compound | compound | 4.27 | 16.31 | 111 | Red |
| Example 119 | 1-20 | C-8 | | | | |
| Comparative | compound | compound | 4.18 | 16.39 | 107 | Red |
| Example 120 | 1-27 | C-8 | | | | |
| Comparative | compound | compound | 4.29 | 15.89 | 114 | Red |
| Example 121 | 1-31 | C-8 | | | | |
| Comparative | compound | compound | 4.22 | 16.52 | 117 | Red |
| Example 122 | 1-52 | C-8 | | | | |
| Comparative | compound | compound | 4.28 | 15.79 | 124 | Red |
| Example 123 | 1-56 | C-8 | | | | |
| Comparative | compound | compound | 4.21 | 15.62 | 124 | Red |
| Example 124 | 1-60 | C-8 | | | | |

Referring to Tables 1 to 3, it was confirmed that the organic light emitting devices of Examples 1 to 145 in which the compound of Chemical Formula 1 and the compound of Chemical Formula 2 were co-deposited as a host for the red light emitting layer had reduced driving voltage and increased efficiency and lifespan compared to Comparative Examples 1 to 124.

From these results, it can be confirmed that the combination of the compound of Chemical Formula 1 and the compound of Chemical Formula 2 is more effective in energy transfer to the dopant in the light emitting layer compared to the combination of Comparative Examples.

[DESCRIPTION OF SYMBOLS]

| | |
|---|---|
| 1: Substrate | 2: Anode |
| 3: Light emitting layer | 4: Cathode |
| 5: Hole injection layer | 6: Hole transport layer |
| 7: Electron transport layer | 8: Electron injection layer |
| 9: Electron blocking layer | 10: Hole blocking layer |
| 11: Electron injection and transport layer | |

1. An organic light emitting device, comprising:
an anode;
a cathode;
and a light emitting layer between the anode and the cathode,
wherein the light emitting layer comprises a compound of the following Chemical Formula 1 and a compound of the following Chemical Formula 2: 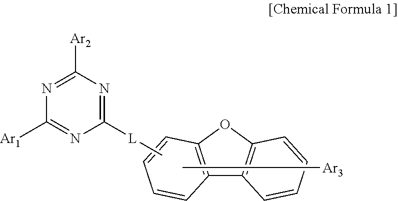
wherein in the Chemical Formula 1:
L is a single bond or substituted or unsubstituted C₆₋₆₀ arylene, arylene;
Ar₁ and Ar₂ are each independently substituted or unsubstituted C₆₋₆₀ aryl, or substituted or unsubstituted C₂₋₆₀ heteroaryl containing at least one heteroatom selected from the group consisting of N, O and S; and
Ar₃ is hydrogen, deuterium, substituted or unsubstituted C₆₋₆₀ aryl, or substituted or unsubstituted C₂₋₆₀ heteroaryl containing at least one heteroatom selected from the group consisting of N, O and S; 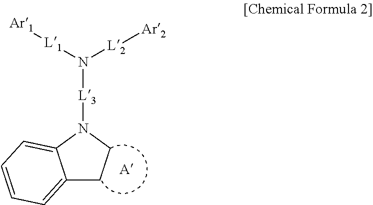
wherein in the Chemical Formula 2:
A′ is a naphthalene ring fused with an adjacent ring and is unsubstituted or substituted with deuterium;
L′₁ and L′₂ are each independently a single bond, substituted or unsubstituted C₆₋₆₀ arylene, or substituted or unsubstituted C₂₋₆₀ heteroarylene containing at least one heteroatom selected from the group consisting of N, O and S;
L′₃ is a substituted or unsubstituted C₆₋₆₀ arylene or substituted or unsubstituted C₂₋₆₀ heteroarylene containing at least one heteroatom selected from the group consisting of N, O and S; and
Ar′₁ and Ar′₂ are each independently a substituted or unsubstituted C₆₋₆₀ aryl or substituted or unsubstituted C₂₋₆₀ heteroaryl containing at least one heteroatom selected from the group consisting of N, O and S.

2. The organic light emitting device of claim 1,
wherein Chemical Formula 1 is the following Chemical Formula 1-1: 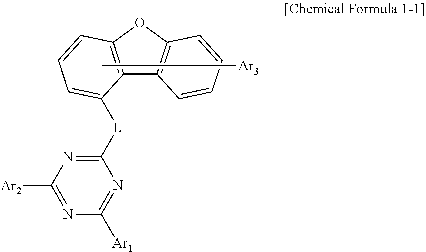
wherein in the Chemical Formula 1-1:
L and Ar₁ to Ar₃ are as defined for Chemical Formula 1.

3. The organic light emitting device of claim 1,
wherein L is a single bond, phenylene, or naphthalenediyl.

4. The organic light emitting device of claim 1,
wherein Ar₁ and Ar₂ are each independently phenyl, biphenylyl, terphenylyl, naphthyl, phenanthrenyl, phenylnaphthyl, naphthylphenyl, dibenzofuranyl, or dibenzothiophenyl.

5. The organic light emitting device of claim 1,
wherein Ar₃ is phenyl, biphenylyl, terphenylyl, naphthyl, phenanthrenyl, phenylnaphthyl, naphthylphenyl, triphenylenyl, dibenzofuranyl, dibenzothiophenyl, benzonaphthofuranyl, benzonaphthothiophenyl, or fluoranthenyl.

6. The organic light emitting device of claim 1,
wherein the compound of Chemical Formula 1 is any one compound selected from the group consisting of the following compounds:

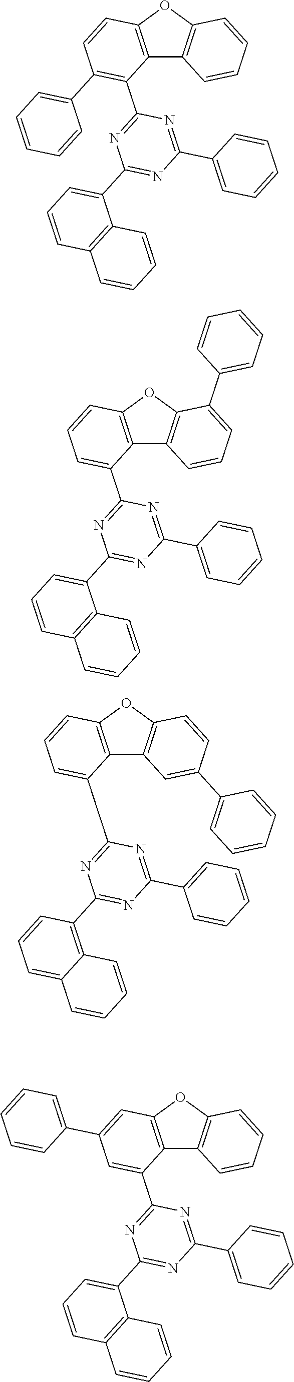

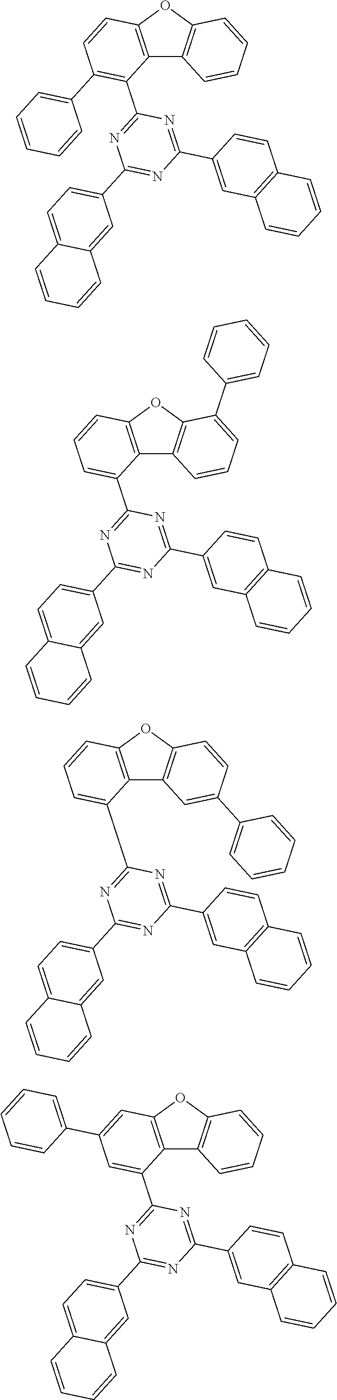

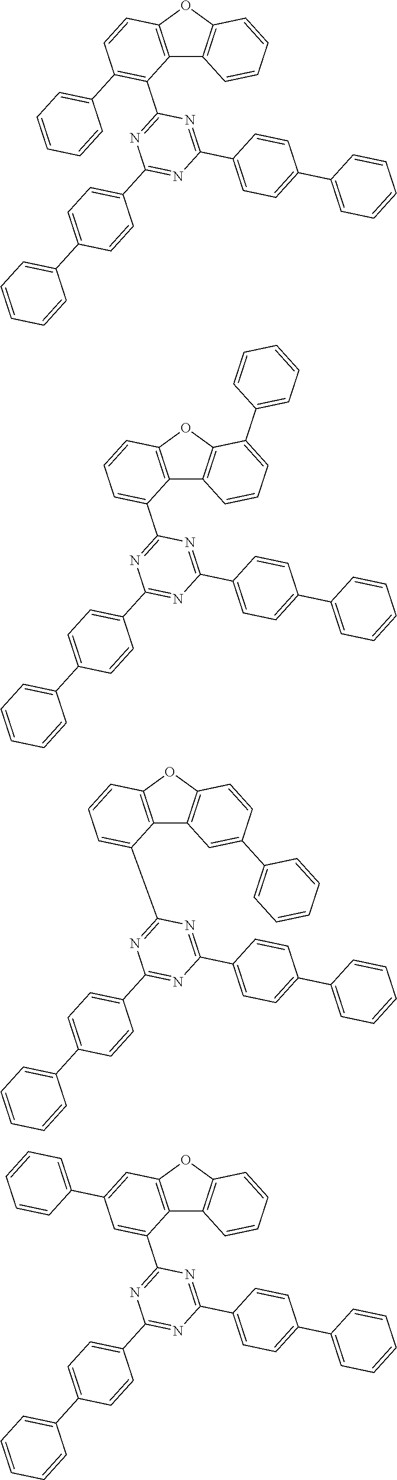

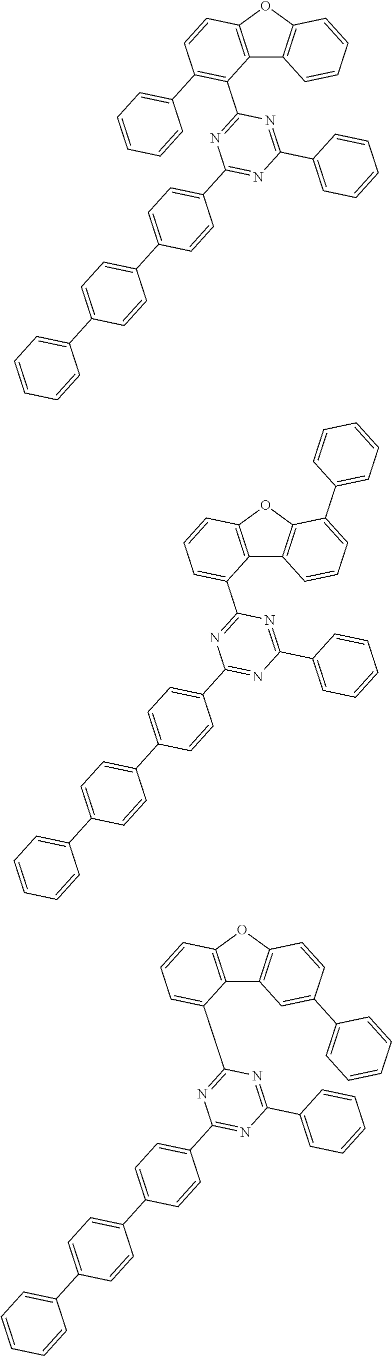 [SMILES: c1ccc(-c2ccc(-c3ccc(-c4nc(-c5ccccc5)nc(-c5c(-c6ccccc6)ccc6oc7ccccc7c56)n4)cc3)cc2)cc1 ; c1ccc(-c2ccc(-c3ccc(-c4nc(-c5ccccc5)nc(-c5cccc6oc7c(-c8ccccc8)cccc7c56)n4)cc3)cc2)cc1 ; c1ccc(-c2ccc(-c3ccc(-c4nc(-c5ccccc5)nc(-c5cccc6oc7ccc(-c8ccccc8)cc7c56)n4)cc3)cc2)cc1]

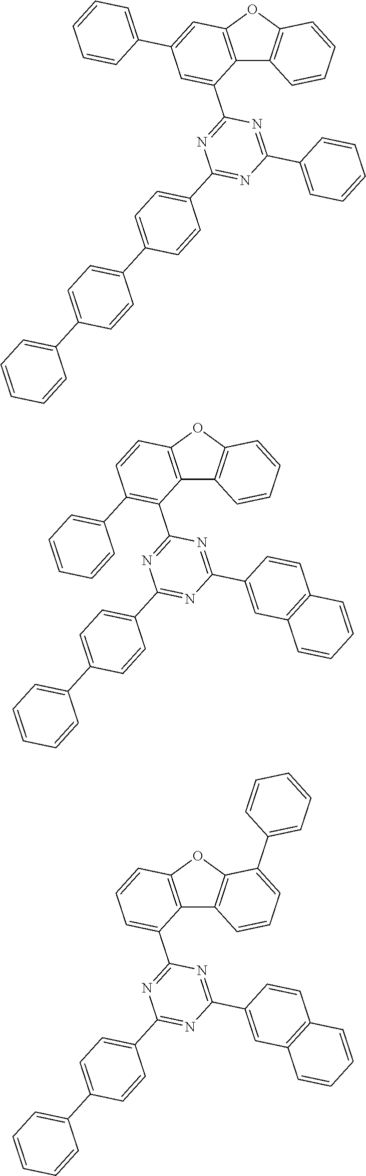 [SMILES: c1ccc(-c2ccc(-c3ccc(-c4nc(-c5ccccc5)nc(-c5cc(-c6ccccc6)cc6oc7ccccc7c56)n4)cc3)cc2)cc1 ; c1ccc(-c2ccc(-c3nc(-c4ccc5ccccc5c4)nc(-c4c(-c5ccccc5)ccc5oc6ccccc6c45)n3)cc2)cc1 ; c1ccc(-c2ccc(-c3nc(-c4ccc5ccccc5c4)nc(-c4cccc5oc6c(-c7ccccc7)cccc6c45)n3)cc2)cc1]

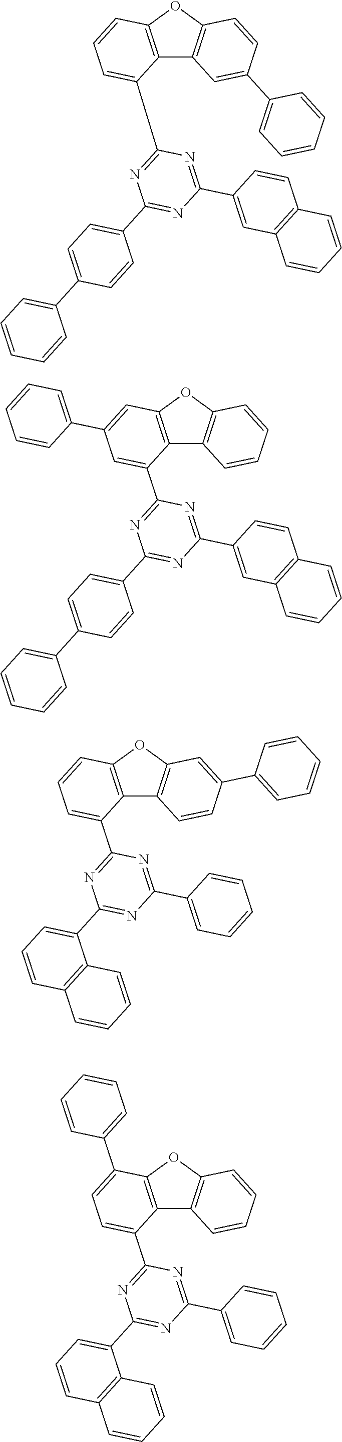

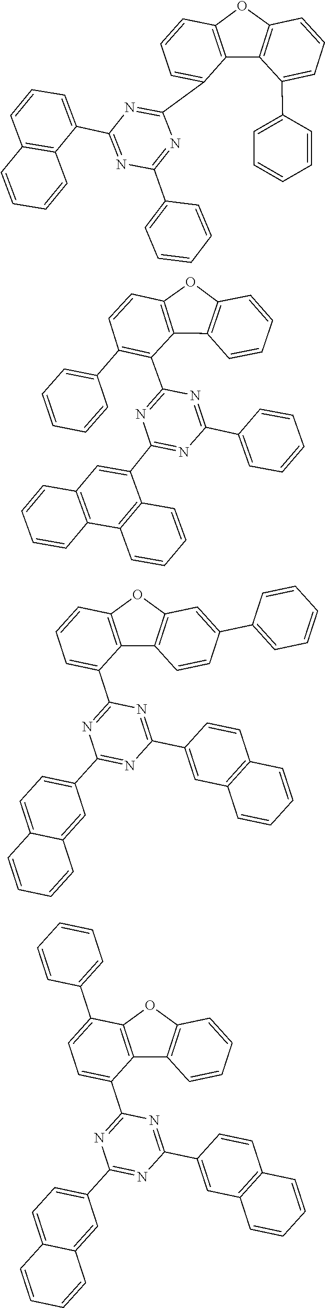

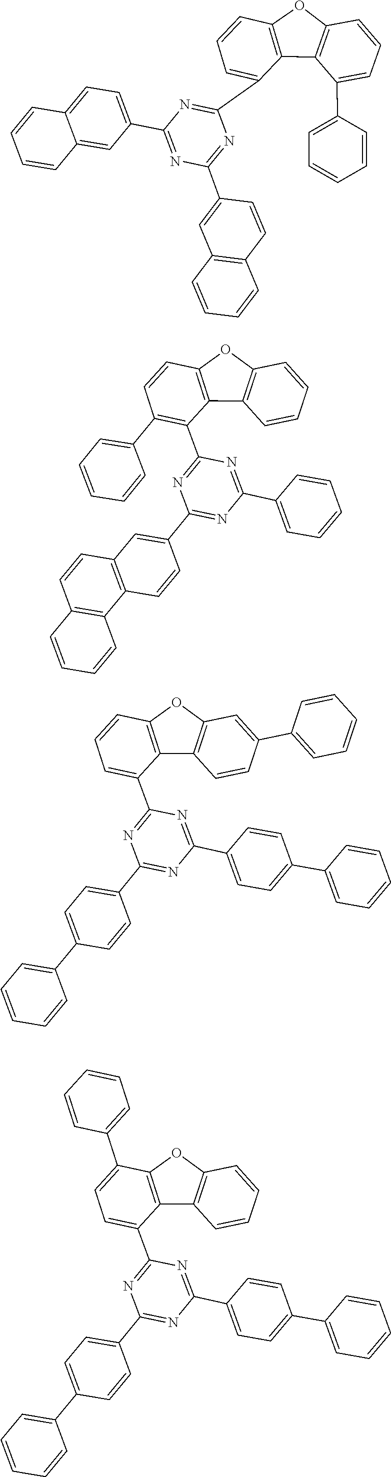

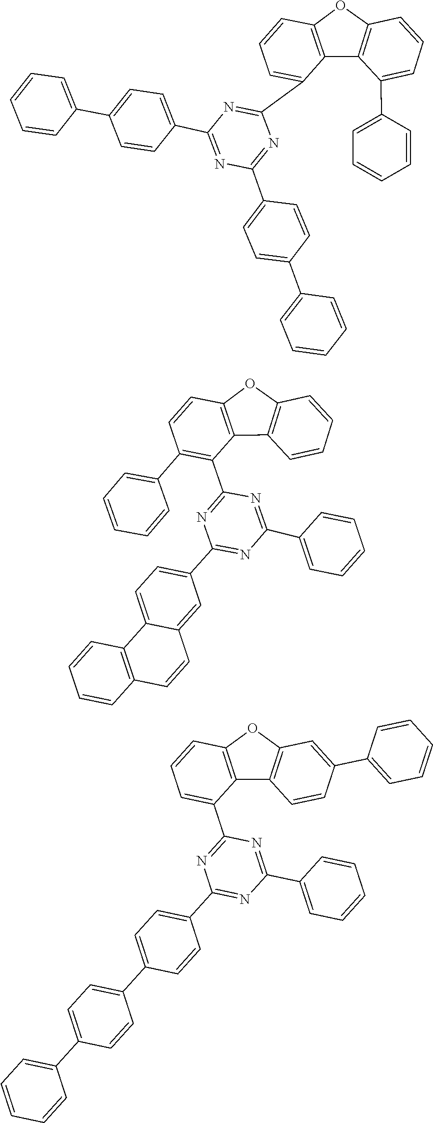

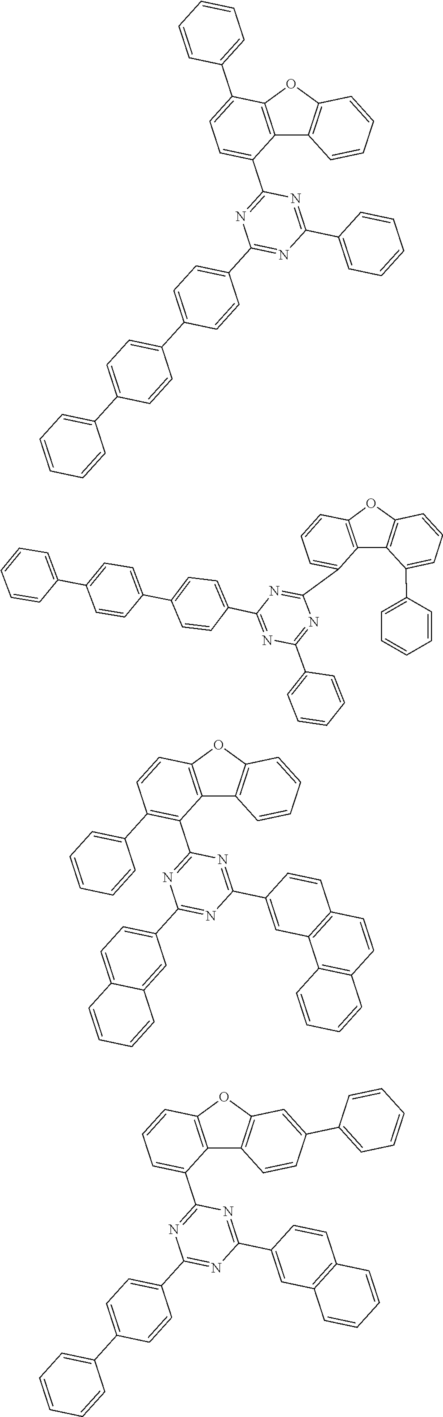

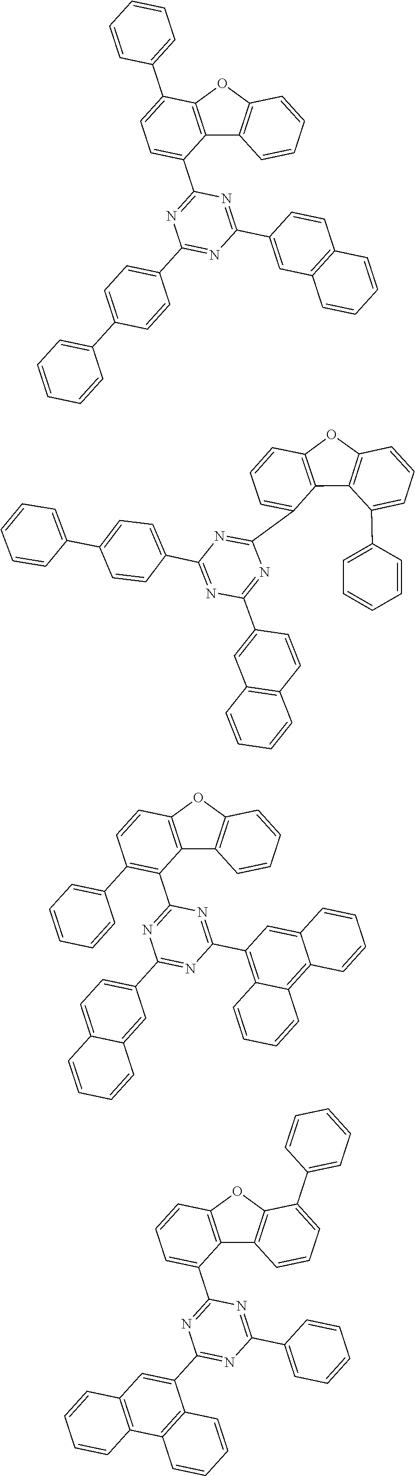 [SMILES: c1ccc(-c2ccc(-c3nc(-c4ccc5ccccc5c4)nc(-c4ccc(-c5ccccc5)c5oc6ccccc6c45)n3)cc2)cc1 ; c1ccc(-c2ccc(-c3nc(-c4ccc5ccccc5c4)nc(-c4cccc5oc6cccc(-c7ccccc7)c6c45)n3)cc2)cc1 ; c1ccc(-c2ccc3oc4ccccc4c3c2-c2nc(-c3ccc4ccccc4c3)nc(-c3cc4ccccc4c4ccccc34)n2)cc1 ; c1ccc(-c2nc(-c3cc4ccccc4c4ccccc34)nc(-c3cccc4oc5c(-c6ccccc6)cccc5c34)n2)cc1]

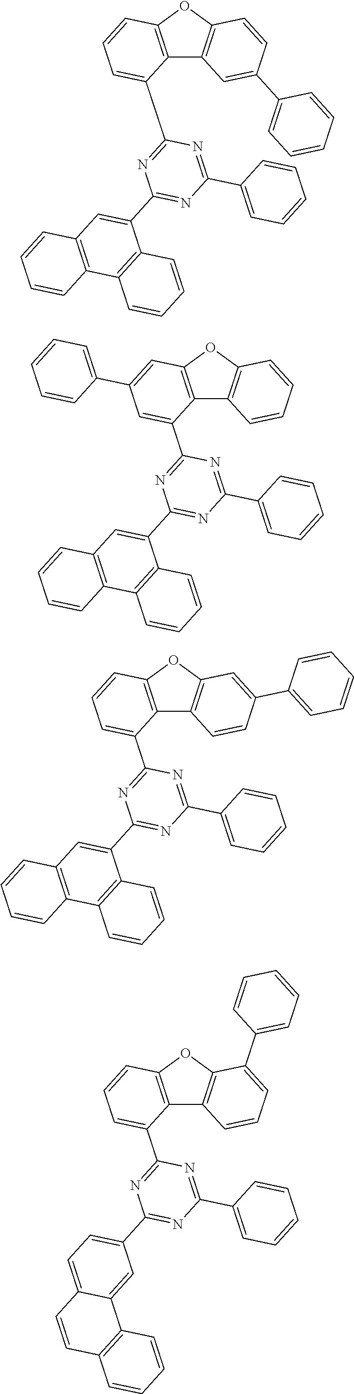

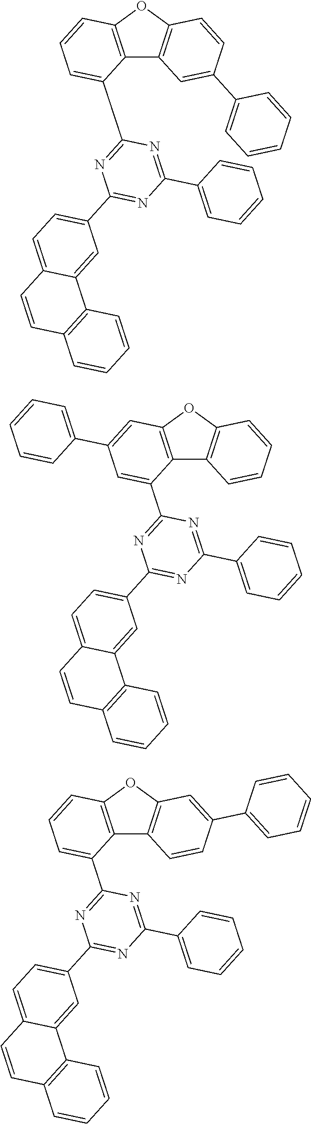

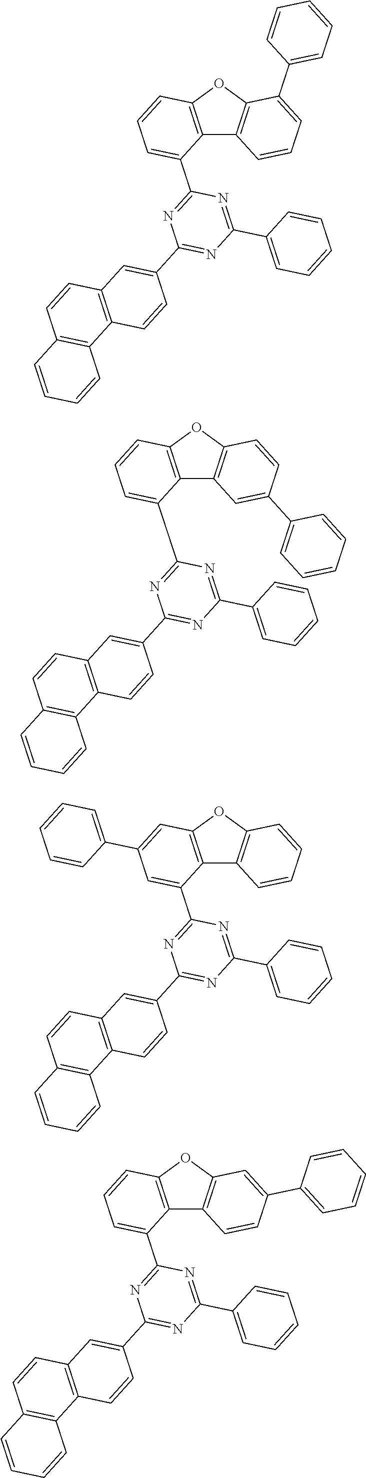

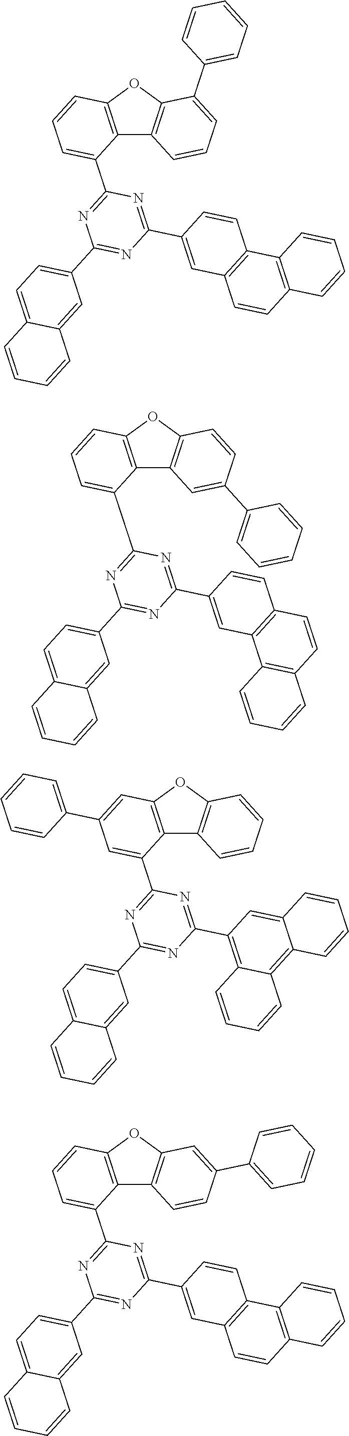

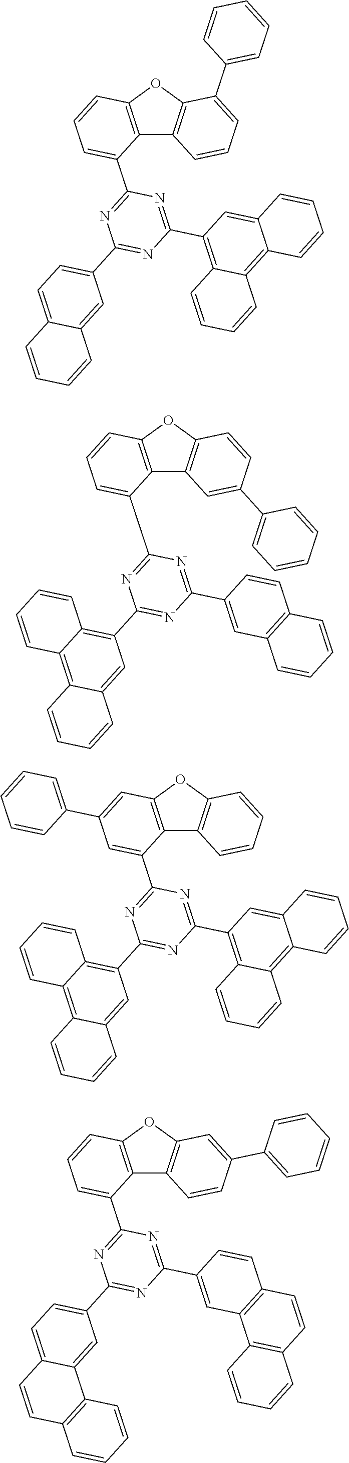

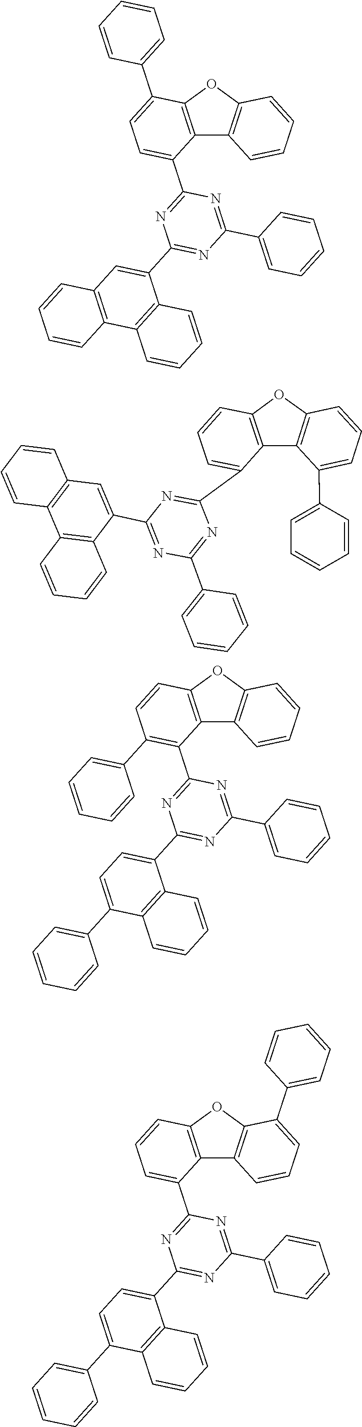

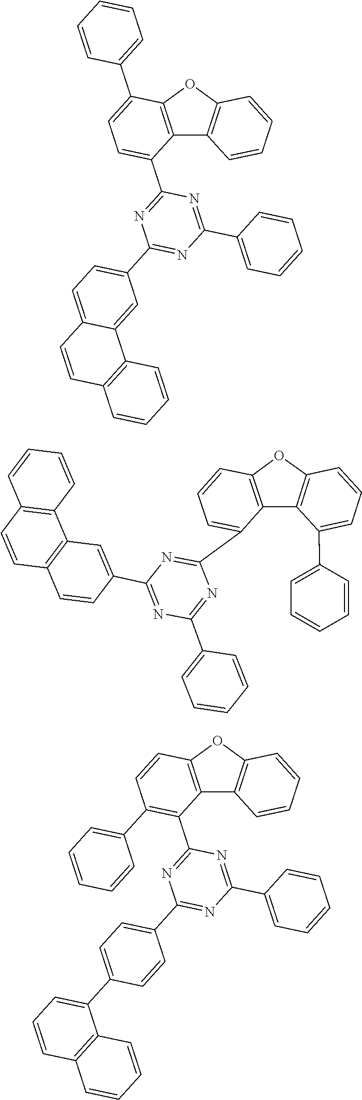

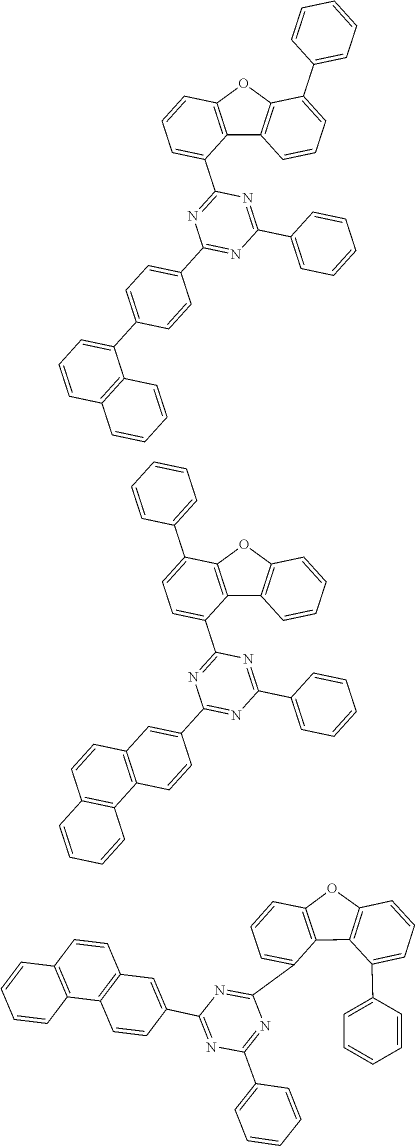

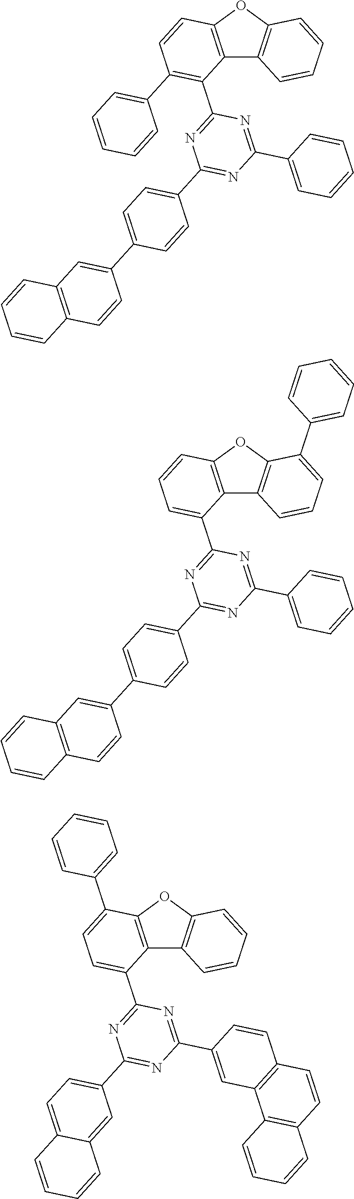

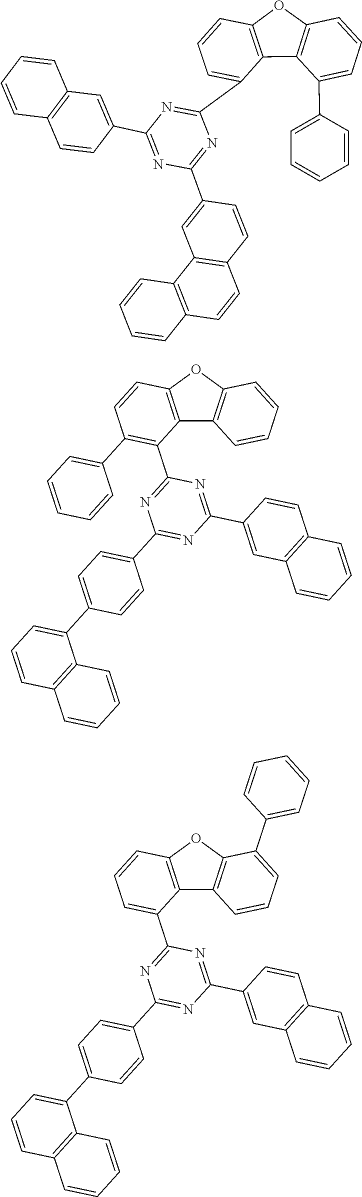

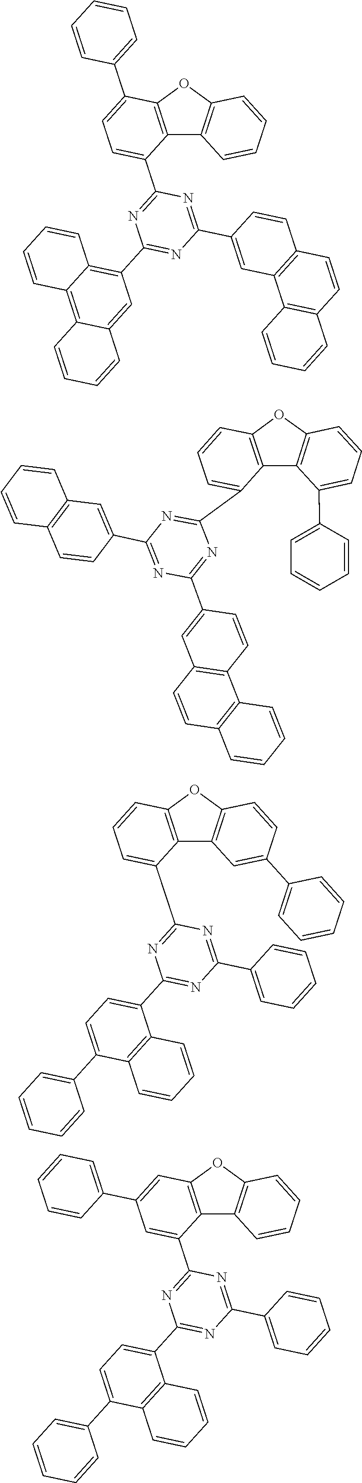

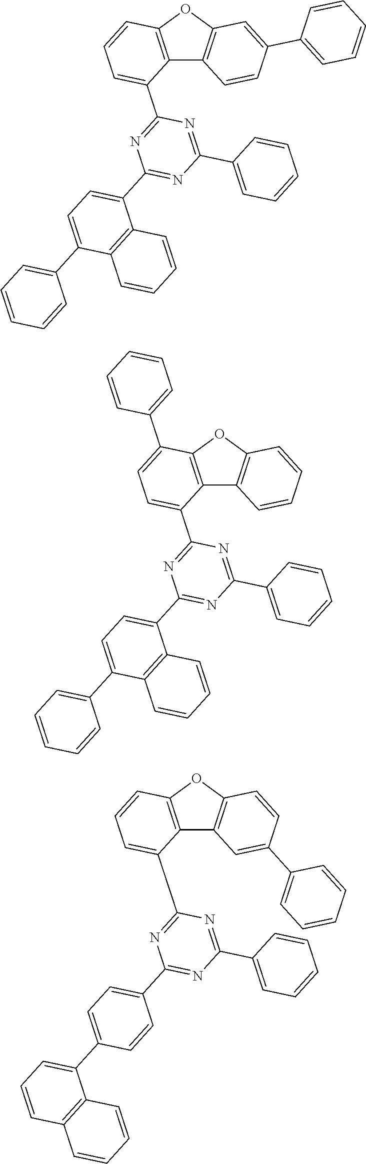 [SMILES: c1ccc(-c2ccc3c(c2)oc2cccc(-c4nc(-c5ccccc5)nc(-c5ccc(-c6ccccc6)c6ccccc56)n4)c23)cc1 ; c1ccc(-c2nc(-c3ccc(-c4ccccc4)c4ccccc34)nc(-c3ccc(-c4ccccc4)c4oc5ccccc5c34)n2)cc1 ; c1ccc(-c2ccc3oc4cccc(-c5nc(-c6ccccc6)nc(-c6ccc(-c7cccc8ccccc78)cc6)n5)c4c3c2)cc1]

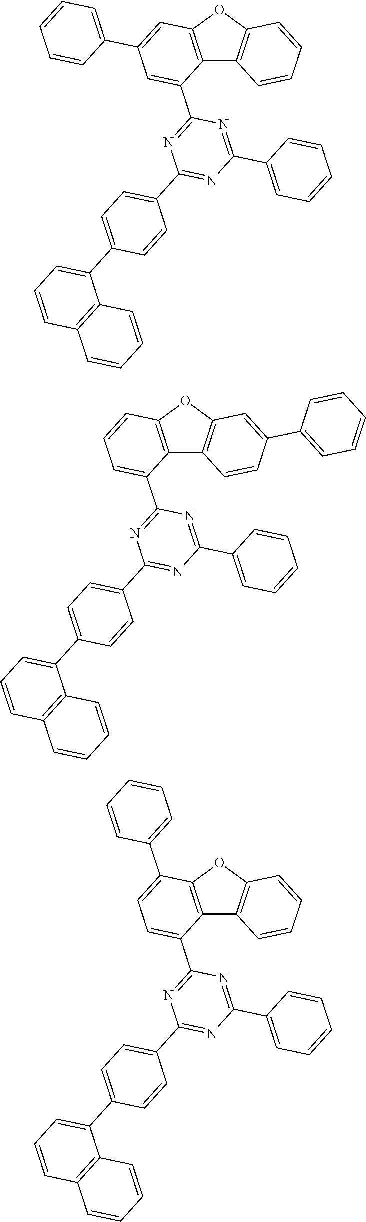

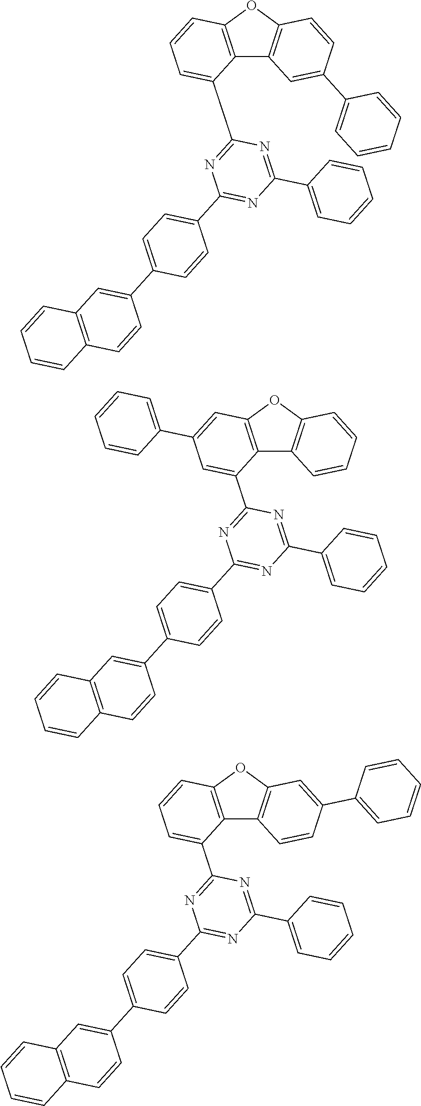

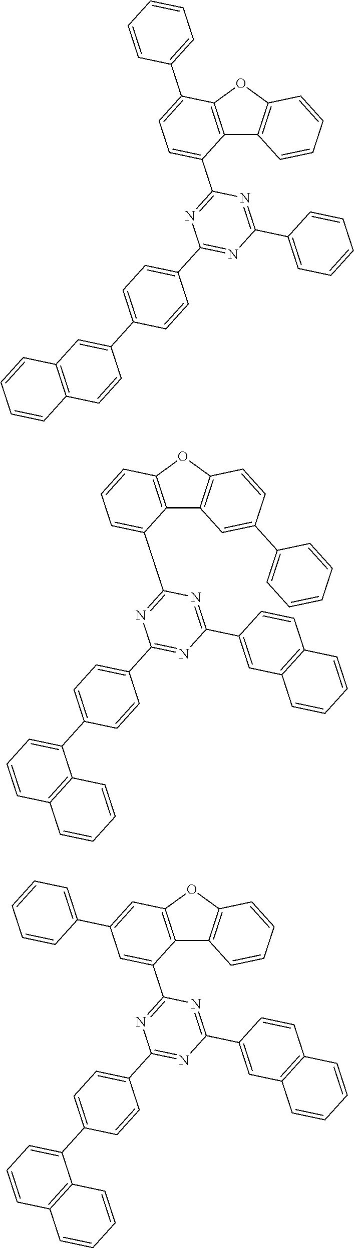 [SMILES: c1ccc(-c2nc(-c3ccc(-c4ccc5ccccc5c4)cc3)nc(-c3ccc(-c4ccccc4)c4oc5ccccc5c34)n2)cc1 ; c1ccc(-c2ccc3oc4cccc(-c5nc(-c6ccc(-c7cccc8ccccc78)cc6)nc(-c6ccc7ccccc7c6)n5)c4c3c2)cc1 ; c1ccc(-c2cc(-c3nc(-c4ccc(-c5cccc6ccccc56)cc4)nc(-c4ccc5ccccc5c4)n3)c3c(c2)oc2ccccc23)cc1]

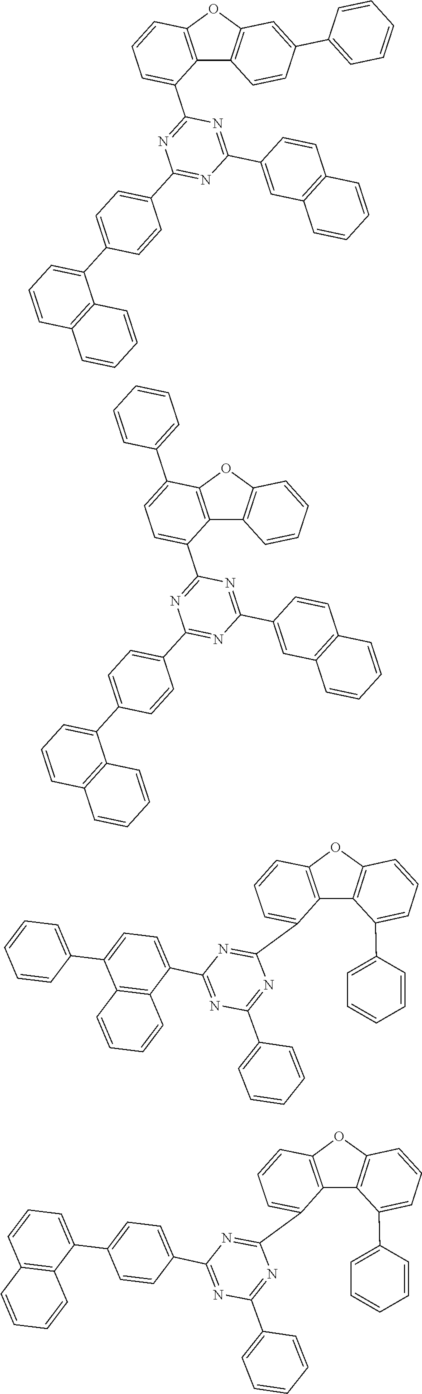

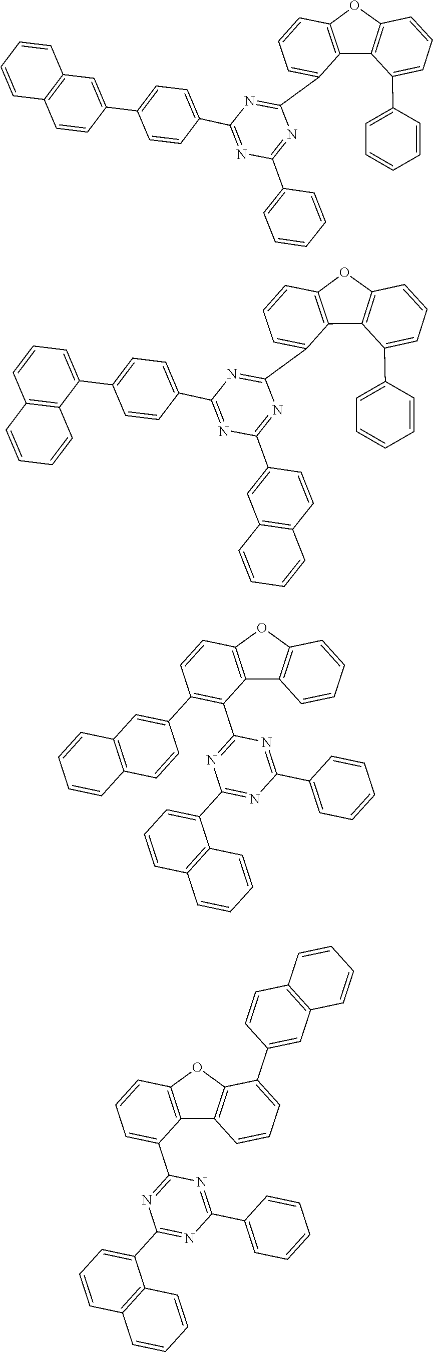

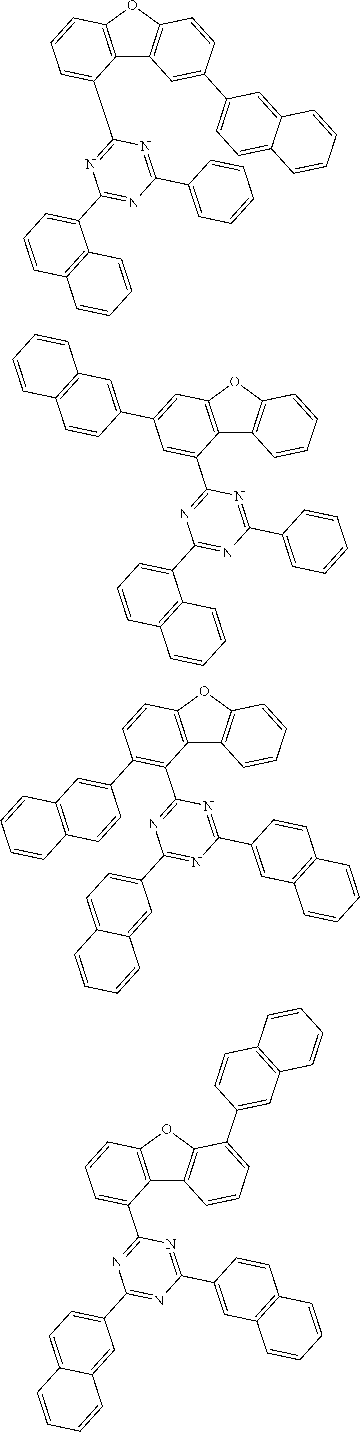

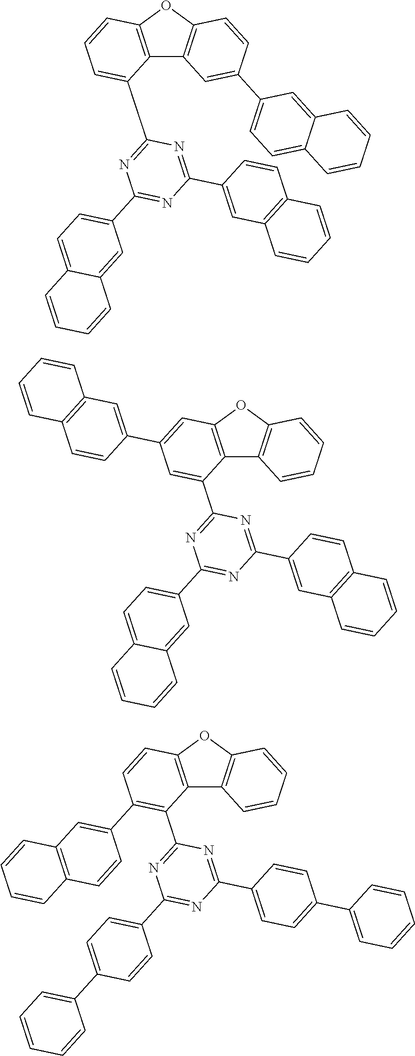

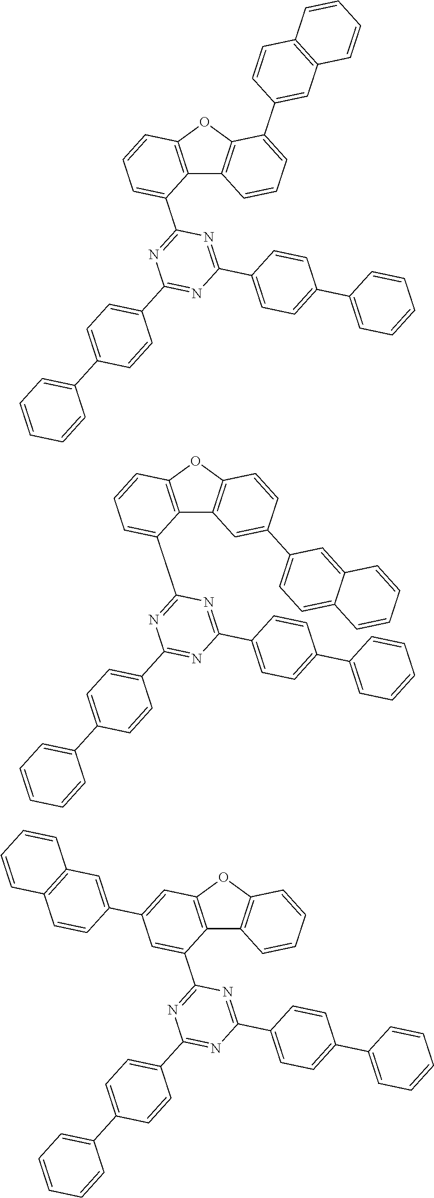

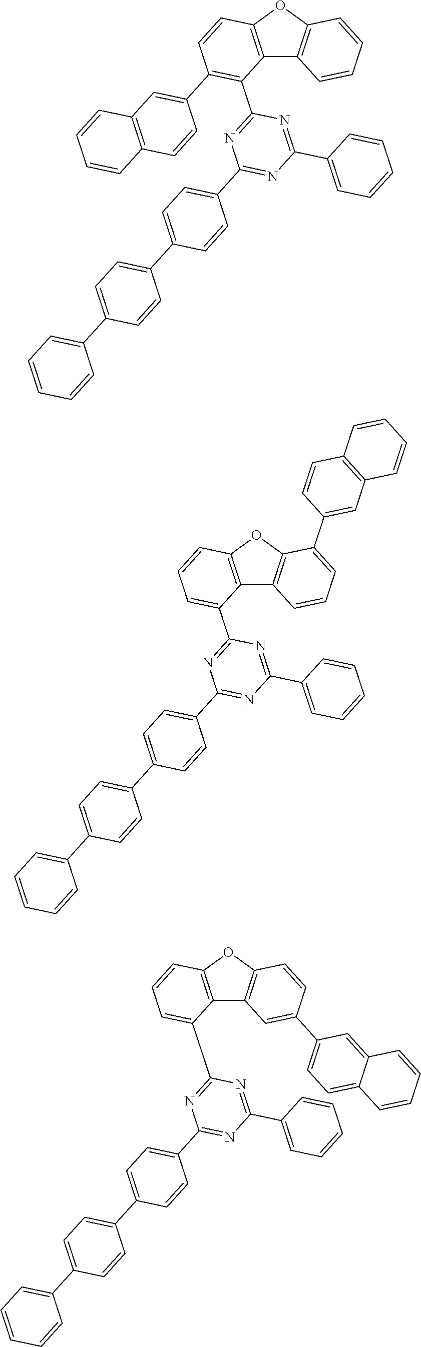 [SMILES: c1ccc(-c2ccc(-c3ccc(-c4nc(-c5ccccc5)nc(-c5c(-c6ccc7ccccc7c6)ccc6oc7ccccc7c56)n4)cc3)cc2)cc1 ; c1ccc(-c2ccc(-c3ccc(-c4nc(-c5ccccc5)nc(-c5cccc6oc7c(-c8ccc9ccccc9c8)cccc7c56)n4)cc3)cc2)cc1 ; c1ccc(-c2ccc(-c3ccc(-c4nc(-c5ccccc5)nc(-c5cccc6oc7ccc(-c8ccc9ccccc9c8)cc7c56)n4)cc3)cc2)cc1]

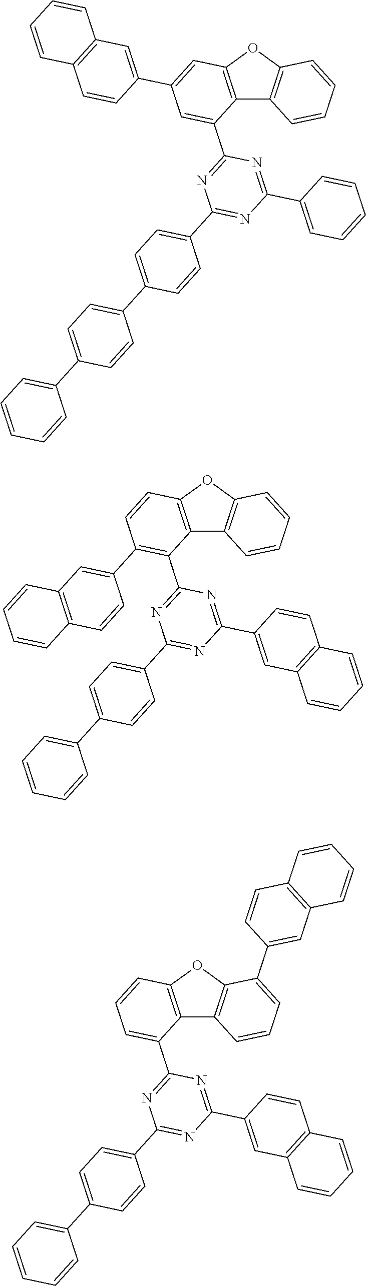 [SMILES: c1ccc(-c2ccc(-c3ccc(-c4nc(-c5ccccc5)nc(-c5cc(-c6ccc7ccccc7c6)cc6oc7ccccc7c56)n4)cc3)cc2)cc1 ; c1ccc(-c2ccc(-c3nc(-c4ccc5ccccc5c4)nc(-c4c(-c5ccc6ccccc6c5)ccc5oc6ccccc6c45)n3)cc2)cc1 ; c1ccc(-c2ccc(-c3nc(-c4ccc5ccccc5c4)nc(-c4cccc5oc6c(-c7ccc8ccccc8c7)cccc6c45)n3)cc2)cc1]

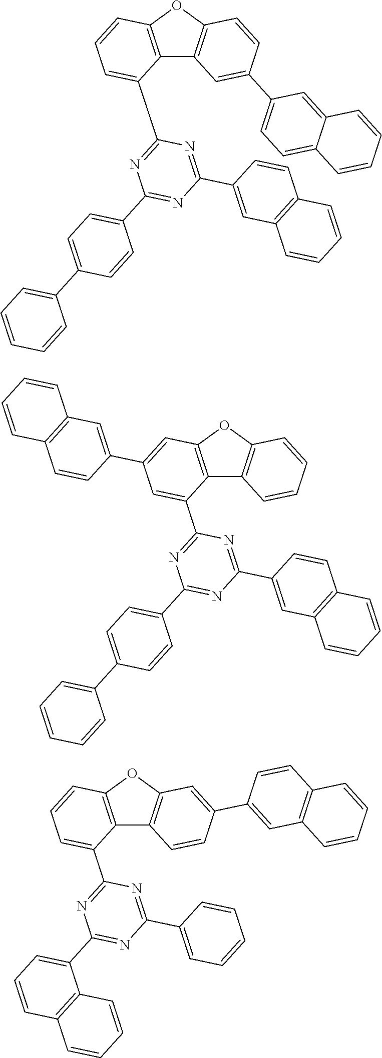

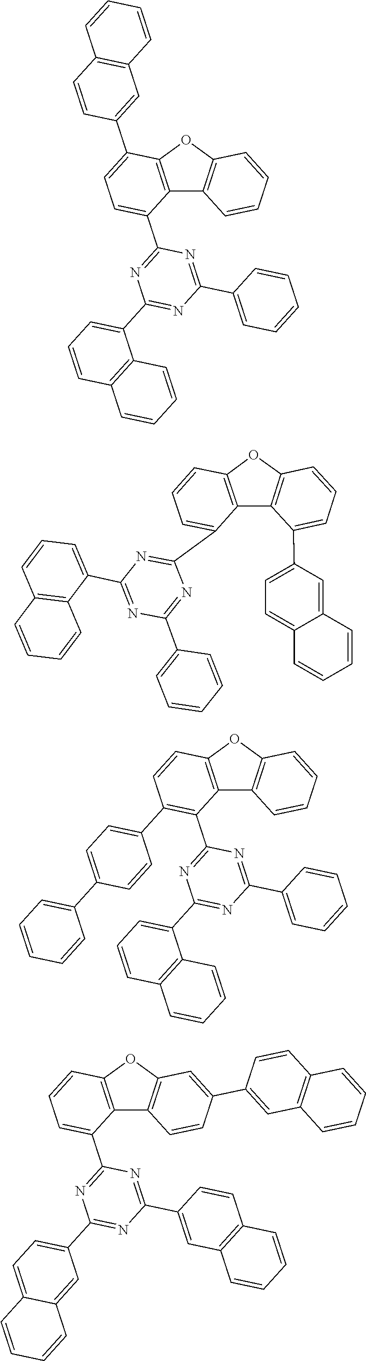

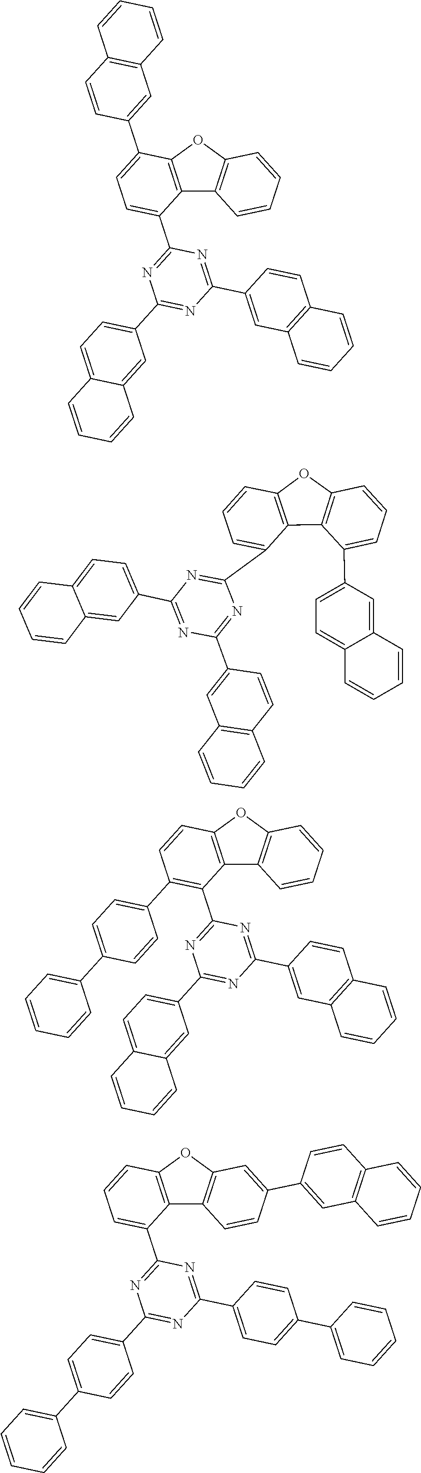

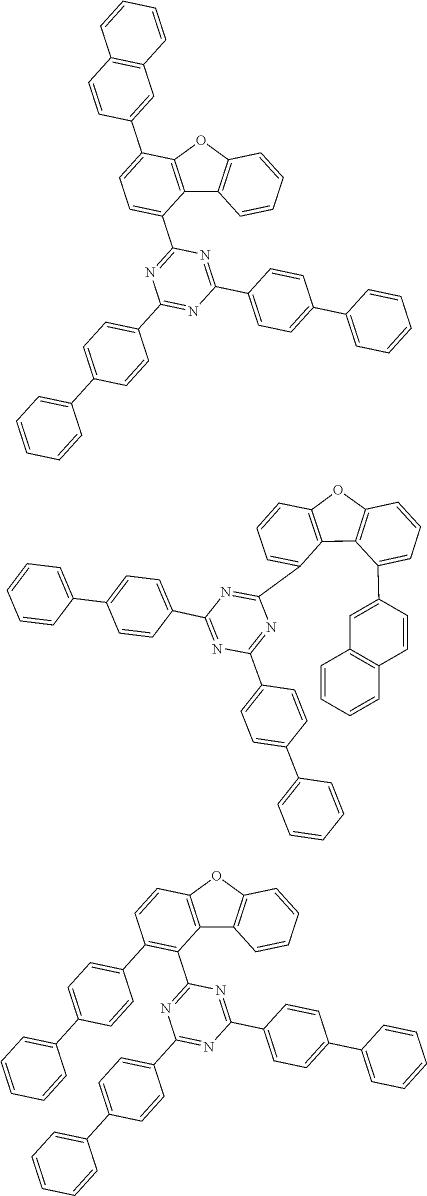 [SMILES: c1ccc(-c2ccc(-c3nc(-c4ccc(-c5ccccc5)cc4)nc(-c4ccc(-c5ccc6ccccc6c5)c5oc6ccccc6c45)n3)cc2)cc1 ; c1ccc(-c2ccc(-c3nc(-c4ccc(-c5ccccc5)cc4)nc(-c4cccc5oc6cccc(-c7ccc8ccccc8c7)c6c45)n3)cc2)cc1 ; c1ccc(-c2ccc(-c3nc(-c4ccc(-c5ccccc5)cc4)nc(-c4c(-c5ccc(-c6ccccc6)cc5)ccc5oc6ccccc6c45)n3)cc2)cc1]

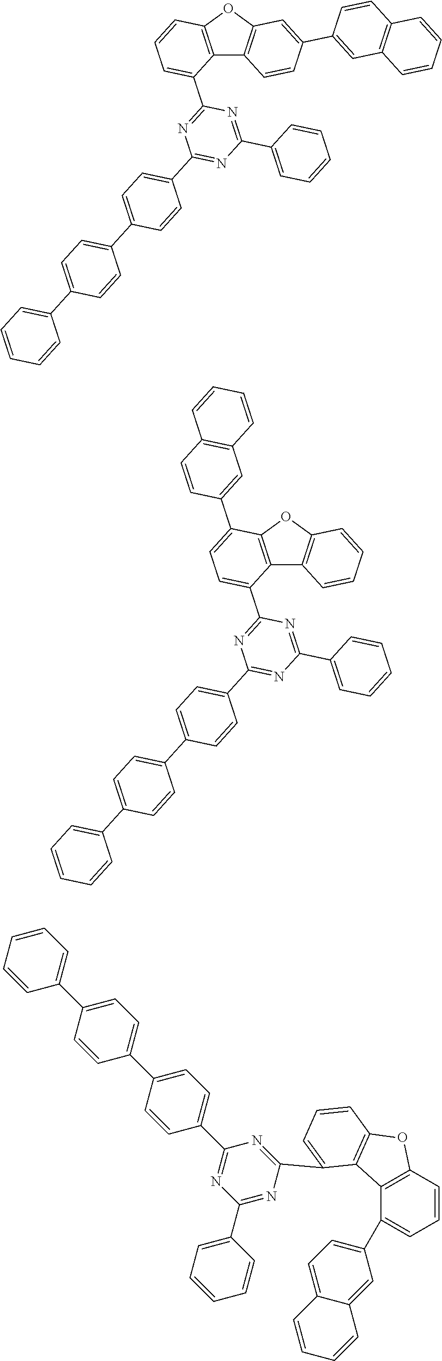 [SMILES: c1ccc(-c2ccc(-c3ccc(-c4nc(-c5ccccc5)nc(-c5cccc6oc7cc(-c8ccc9ccccc9c8)ccc7c56)n4)cc3)cc2)cc1 ; c1ccc(-c2ccc(-c3ccc(-c4nc(-c5ccccc5)nc(-c5ccc(-c6ccc7ccccc7c6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9ccccc9c8)c7c56)n4)cc3)cc2)cc1]

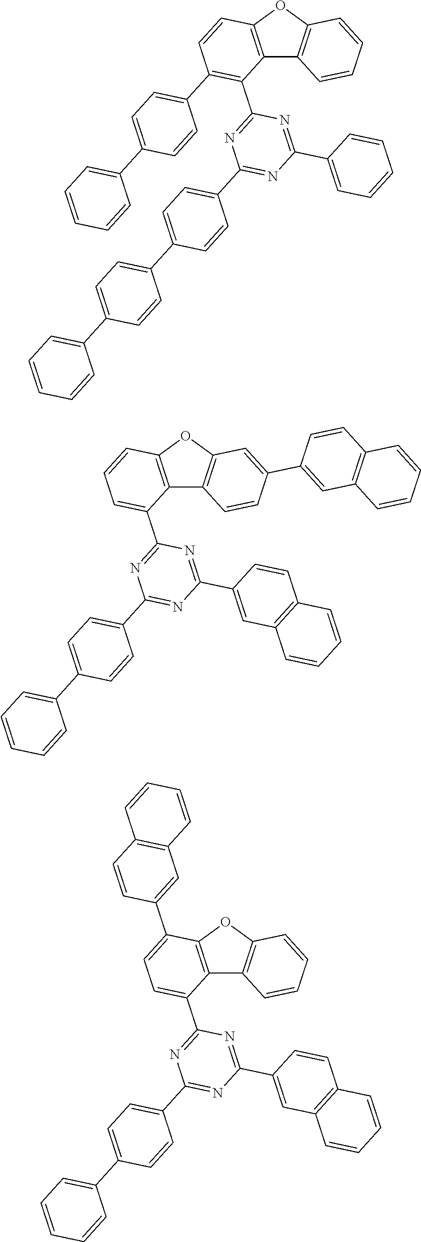

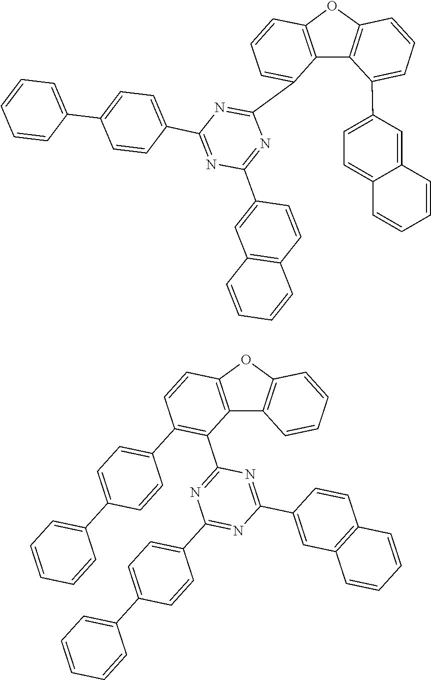

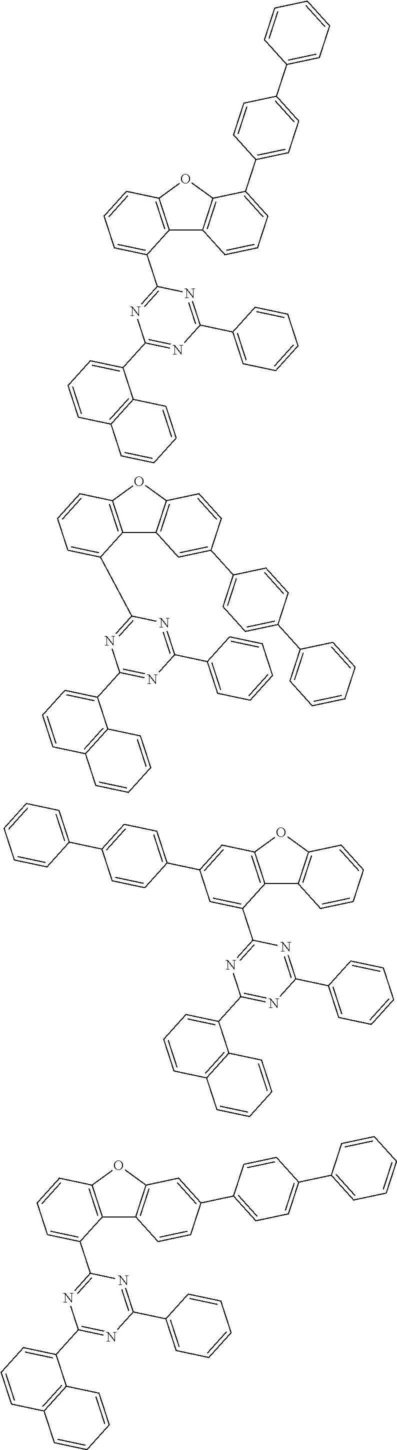

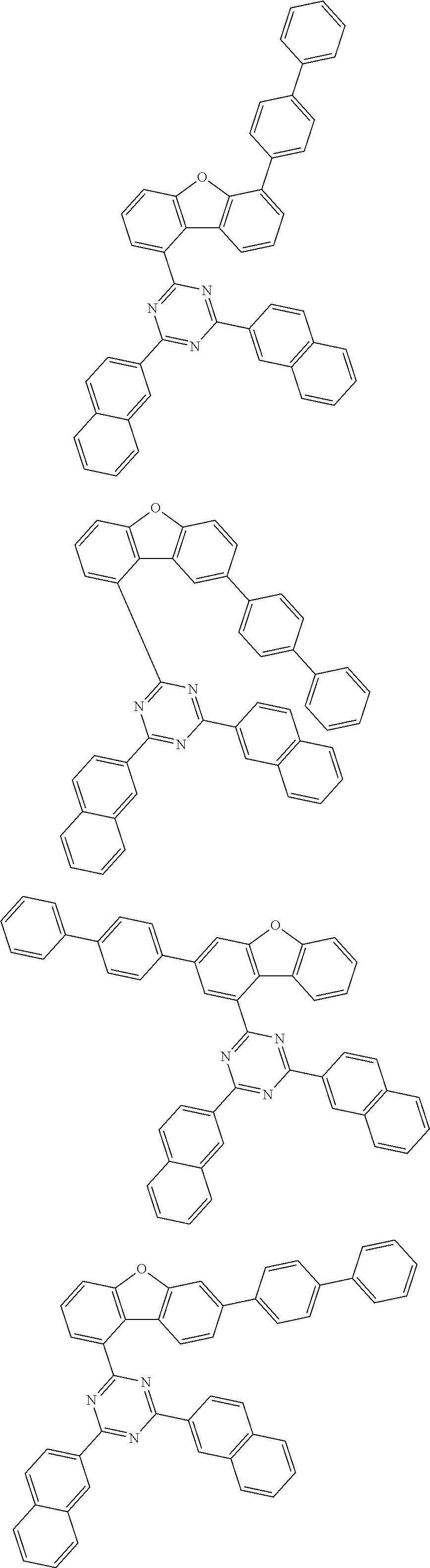

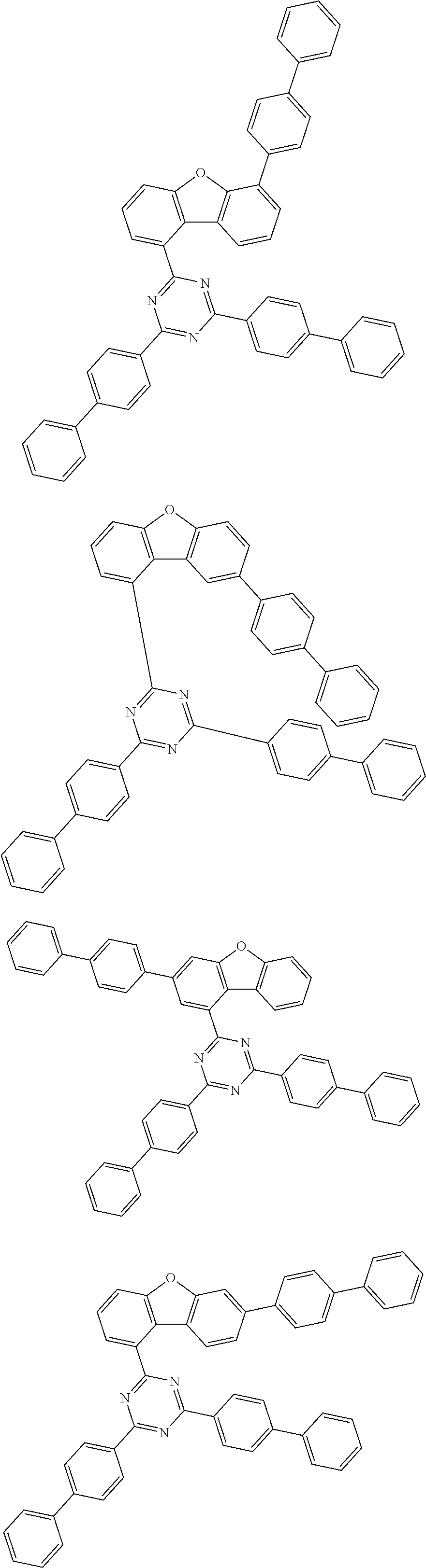

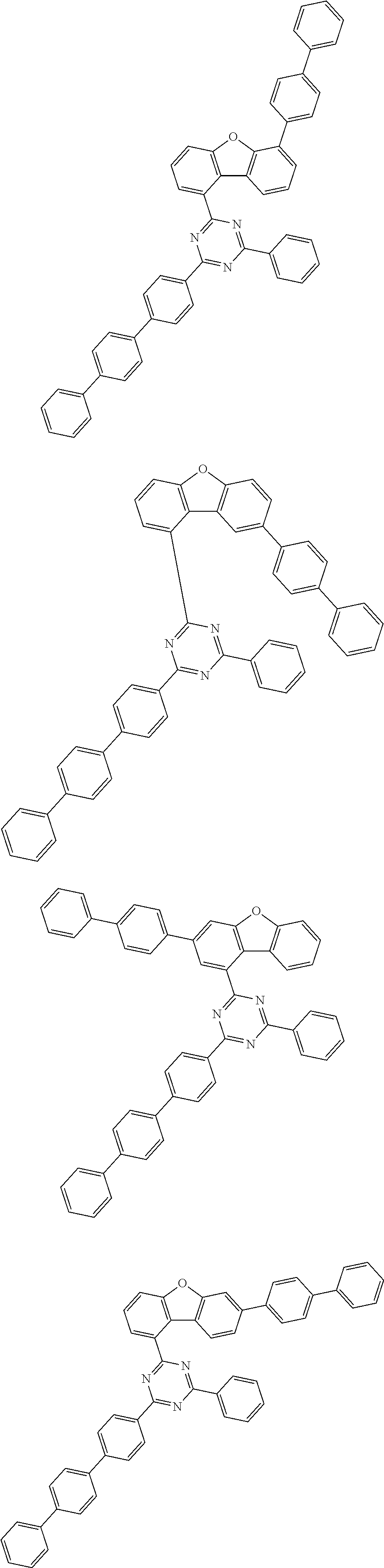

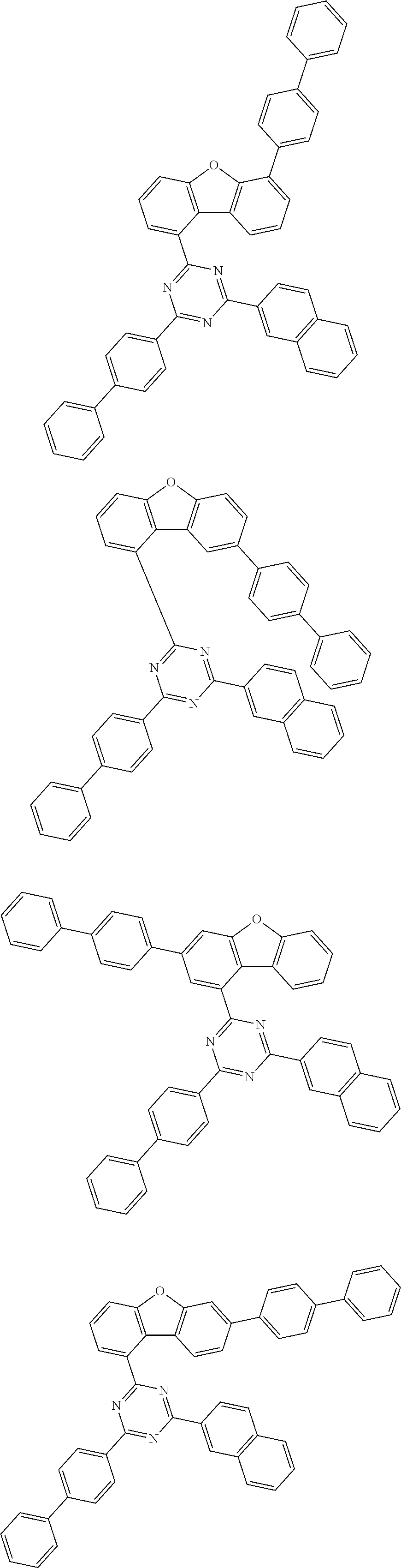 [SMILES: c1ccc(-c2ccc(-c3nc(-c4ccc5ccccc5c4)nc(-c4cccc5oc6c(-c7ccc(-c8ccccc8)cc7)cccc6c45)n3)cc2)cc1 ; c1ccc(-c2ccc(-c3ccc4oc5cccc(-c6nc(-c7ccc(-c8ccccc8)cc7)nc(-c7ccc8ccccc8c7)n6)c5c4c3)cc2)cc1 ; c1ccc(-c2ccc(-c3cc(-c4nc(-c5ccc(-c6ccccc6)cc5)nc(-c5ccc6ccccc6c5)n4)c4c(c3)oc3ccccc34)cc2)cc1 ; c1ccc(-c2ccc(-c3ccc4c(c3)oc3cccc(-c5nc(-c6ccc(-c7ccccc7)cc6)nc(-c6ccc7ccccc7c6)n5)c34)cc2)cc1]

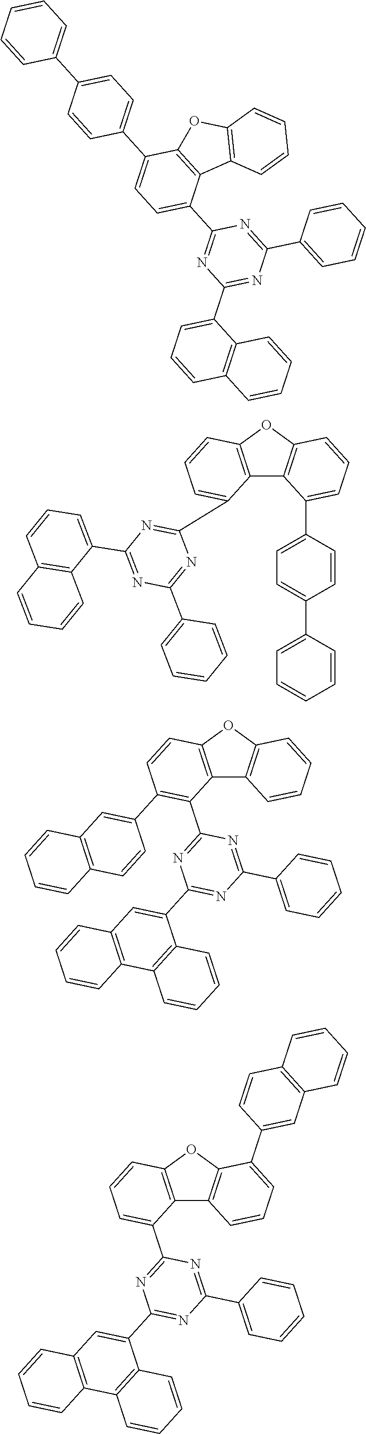

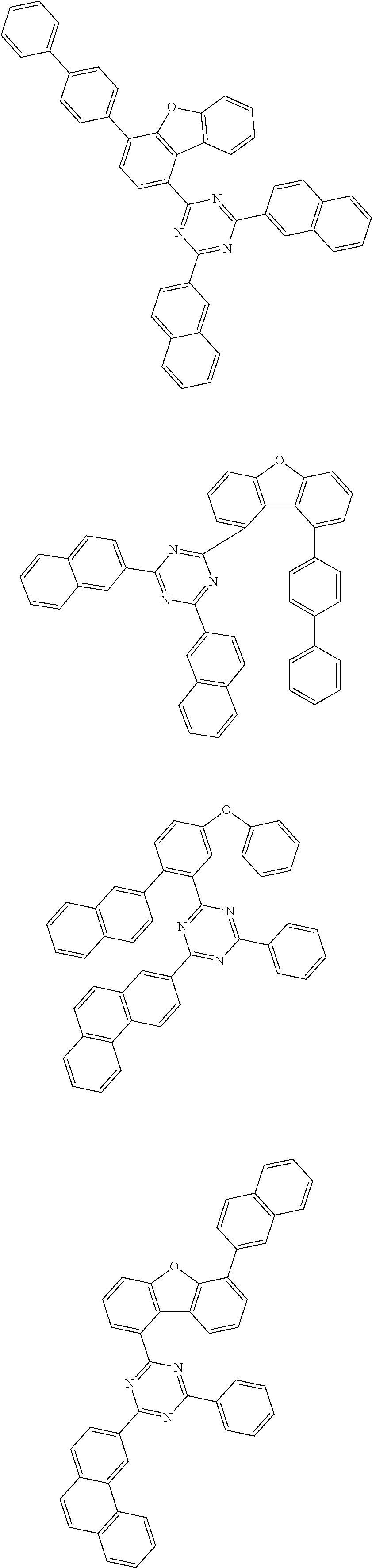 [SMILES: c1ccc(-c2ccc(-c3ccc(-c4nc(-c5ccc6ccccc6c5)nc(-c5ccc6ccccc6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccccc8c7)n6)c5c34)cc2)cc1 ; c1ccc(-c2nc(-c3ccc4c(ccc5ccccc54)c3)nc(-c3c(-c4ccc5ccccc5c4)ccc4oc5ccccc5c34)n2)cc1 ; c1ccc(-c2nc(-c3ccc4ccc5ccccc5c4c3)nc(-c3cccc4oc5c(-c6ccc7ccccc7c6)cccc5c34)n2)cc1]

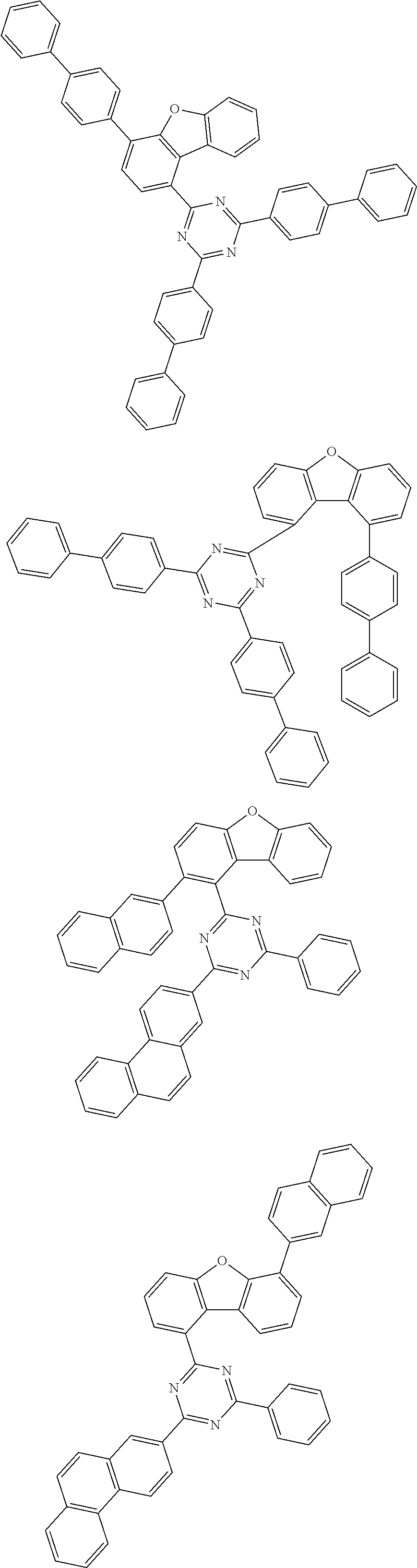

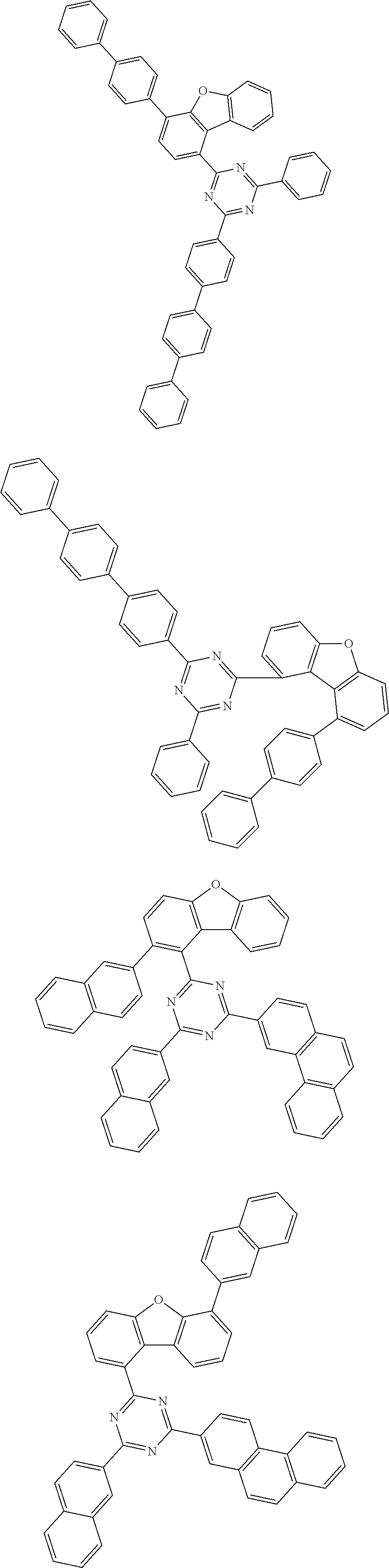 [SMILES: c1ccc(-c2ccc(-c3ccc(-c4nc(-c5ccccc5)nc(-c5ccc(-c6ccc(-c7ccccc7)cc6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc(-c9ccccc9)cc8)c7c56)n4)cc3)cc2)cc1 ; c1ccc2cc(-c3nc(-c4ccc5ccc6ccccc6c5c4)nc(-c4c(-c5ccc6ccccc6c5)ccc5oc6ccccc6c45)n3)ccc2c1 ; c1ccc2cc(-c3nc(-c4ccc5c(ccc6ccccc65)c4)nc(-c4cccc5oc6c(-c7ccc8ccccc8c7)cccc6c45)n3)ccc2c1]

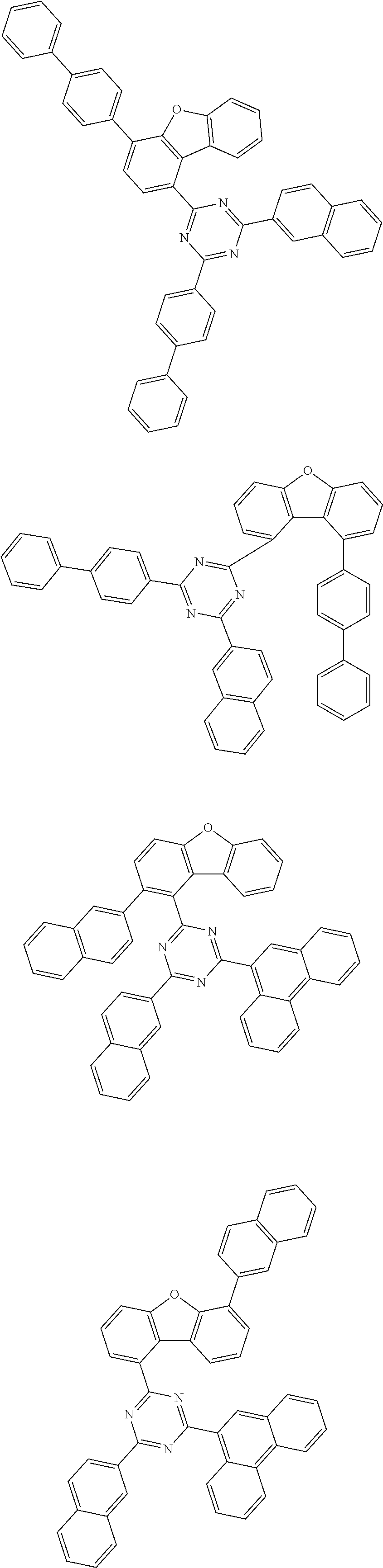 [SMILES: c1ccc(-c2ccc(-c3nc(-c4ccc5ccccc5c4)nc(-c4ccc(-c5ccc(-c6ccccc6)cc5)c5oc6ccccc6c45)n3)cc2)cc1 ; c1ccc(-c2ccc(-c3nc(-c4ccc5ccccc5c4)nc(-c4cccc5oc6cccc(-c7ccc(-c8ccccc8)cc7)c6c45)n3)cc2)cc1 ; c1ccc2cc(-c3nc(-c4cc5ccccc5c5ccccc45)nc(-c4c(-c5ccc6ccccc6c5)ccc5oc6ccccc6c45)n3)ccc2c1 ; c1ccc2cc(-c3nc(-c4cc5ccccc5c5ccccc45)nc(-c4cccc5oc6c(-c7ccc8ccccc8c7)cccc6c45)n3)ccc2c1]

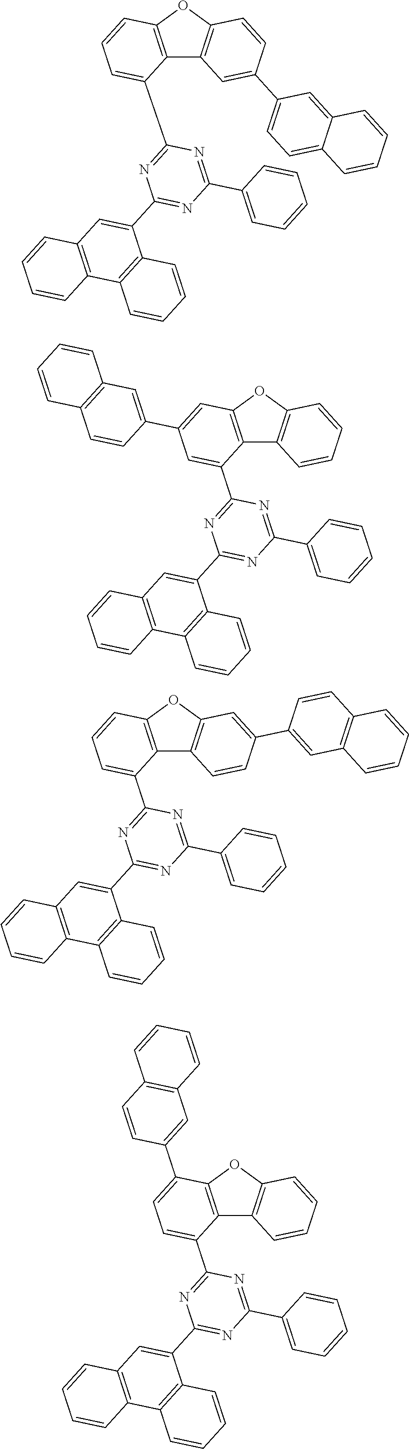

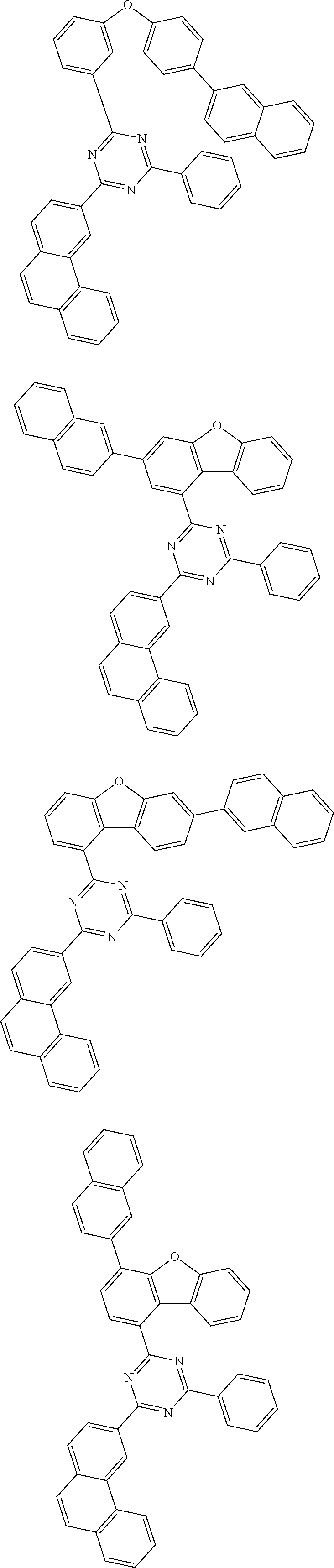 [SMILES: c1ccc(-c2nc(-c3ccc4ccc5ccccc5c4c3)nc(-c3cccc4oc5ccc(-c6ccc7ccccc7c6)cc5c34)n2)cc1 ; c1ccc(-c2nc(-c3ccc4ccc5ccccc5c4c3)nc(-c3cc(-c4ccc5ccccc5c4)cc4oc5ccccc5c34)n2)cc1 ; c1ccc(-c2nc(-c3ccc4ccc5ccccc5c4c3)nc(-c3cccc4oc5cc(-c6ccc7ccccc7c6)ccc5c34)n2)cc1 ; c1ccc(-c2nc(-c3ccc4ccc5ccccc5c4c3)nc(-c3ccc(-c4ccc5ccccc5c4)c4oc5ccccc5c34)n2)cc1]

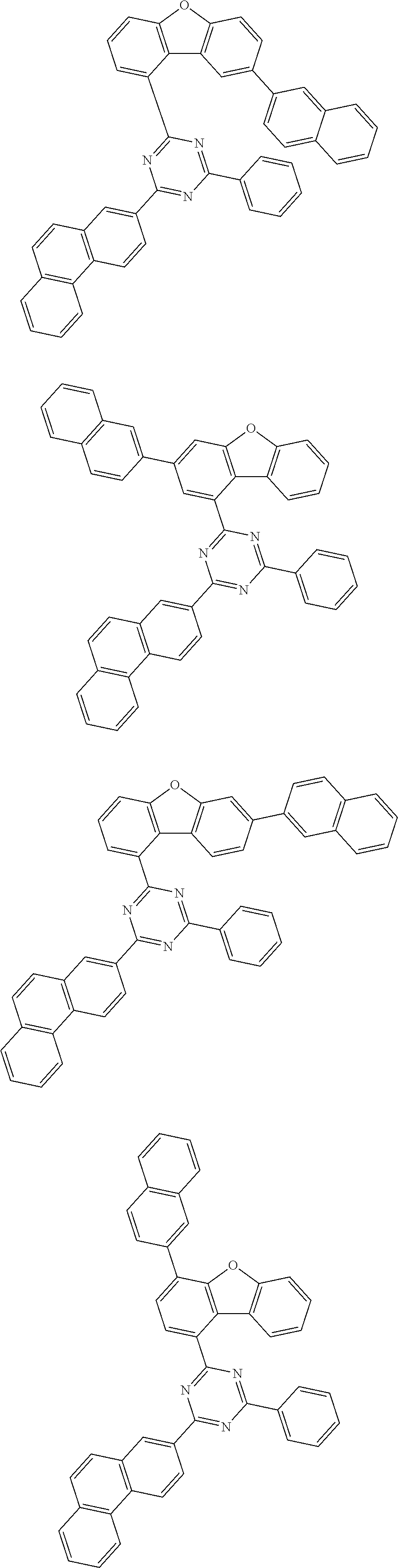 [SMILES: c1ccc(-c2nc(-c3ccc4c(ccc5ccccc54)c3)nc(-c3cccc4oc5ccc(-c6ccc7ccccc7c6)cc5c34)n2)cc1 ; c1ccc(-c2nc(-c3ccc4c(ccc5ccccc54)c3)nc(-c3cc(-c4ccc5ccccc5c4)cc4oc5ccccc5c34)n2)cc1 ; c1ccc(-c2nc(-c3ccc4c(ccc5ccccc54)c3)nc(-c3cccc4oc5cc(-c6ccc7ccccc7c6)ccc5c34)n2)cc1 ; c1ccc(-c2nc(-c3ccc4c(ccc5ccccc54)c3)nc(-c3ccc(-c4ccc5ccccc5c4)c4oc5ccccc5c34)n2)cc1]

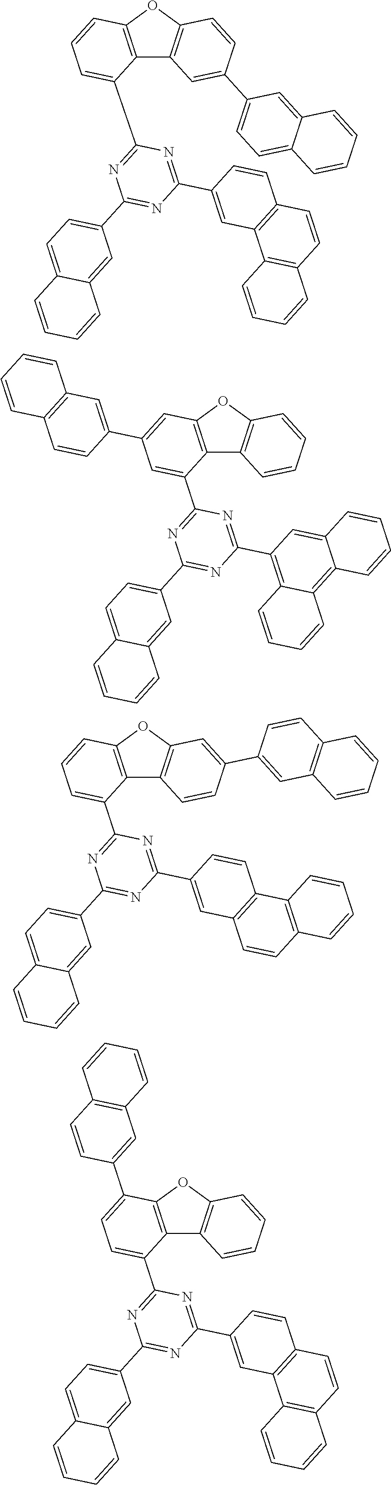

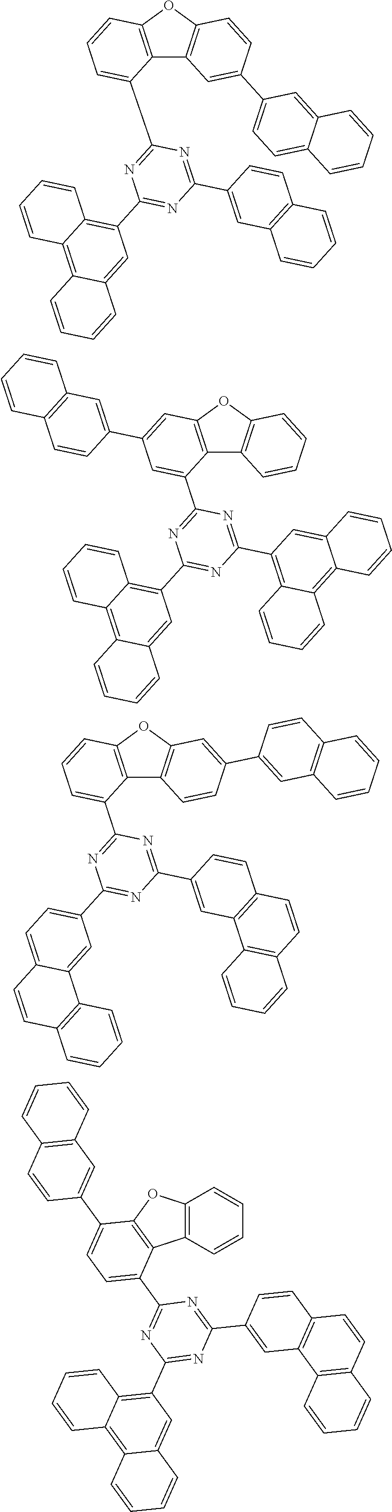

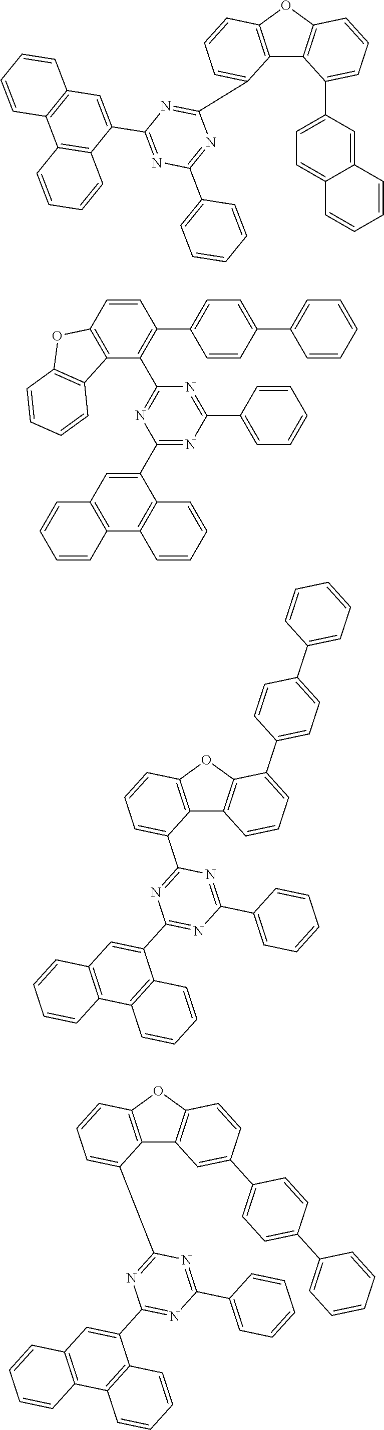

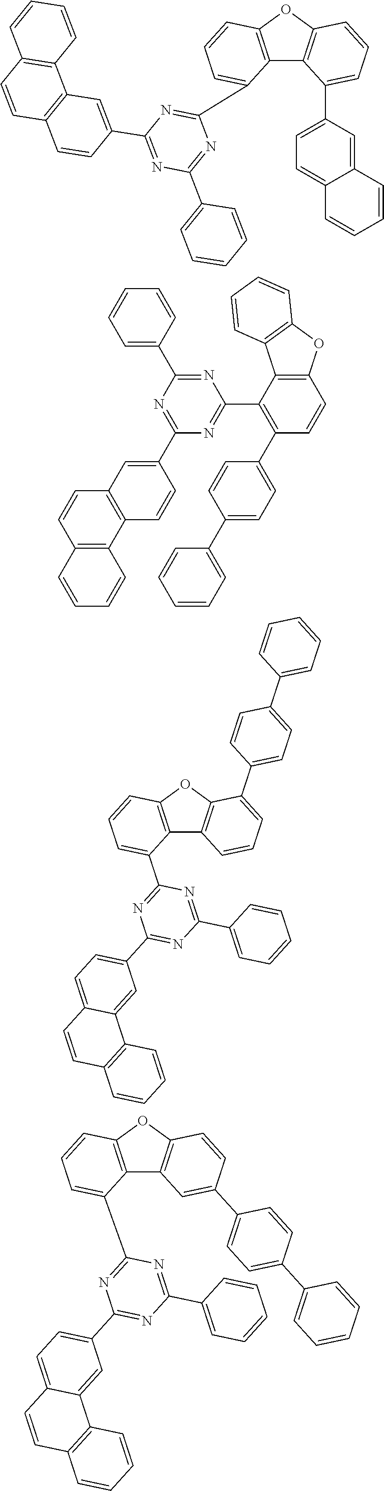

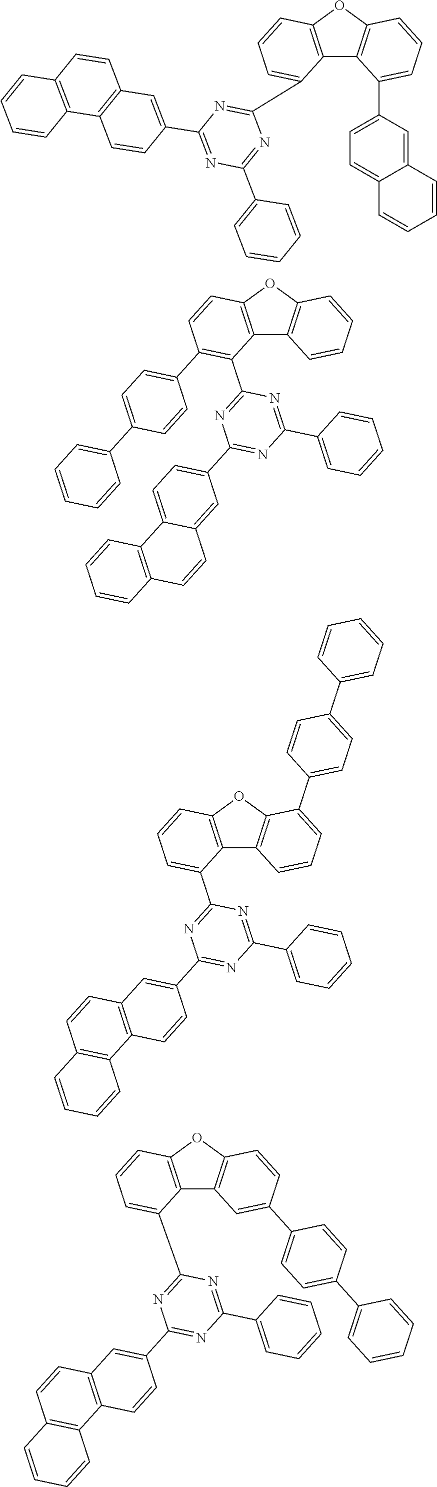

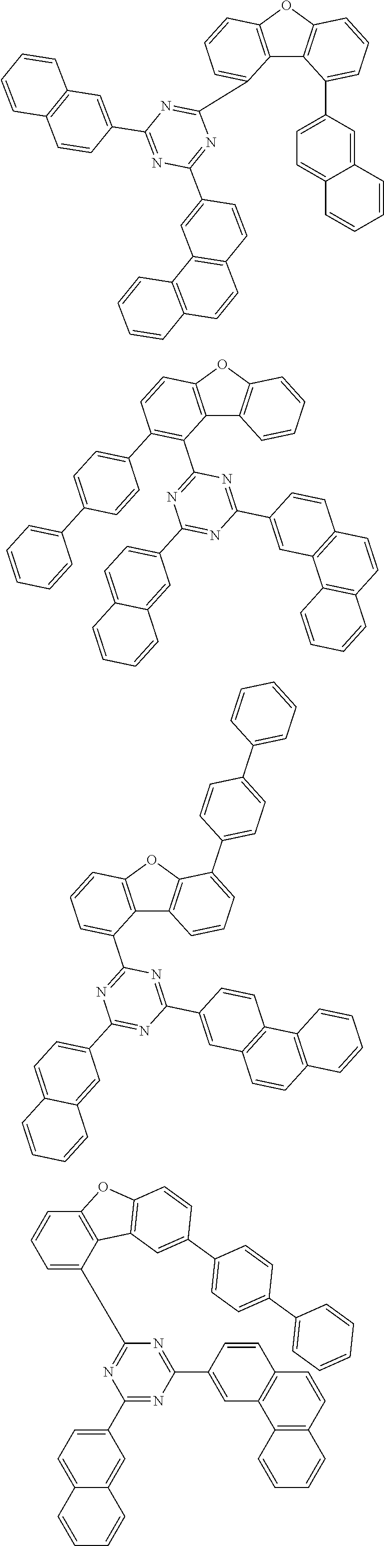

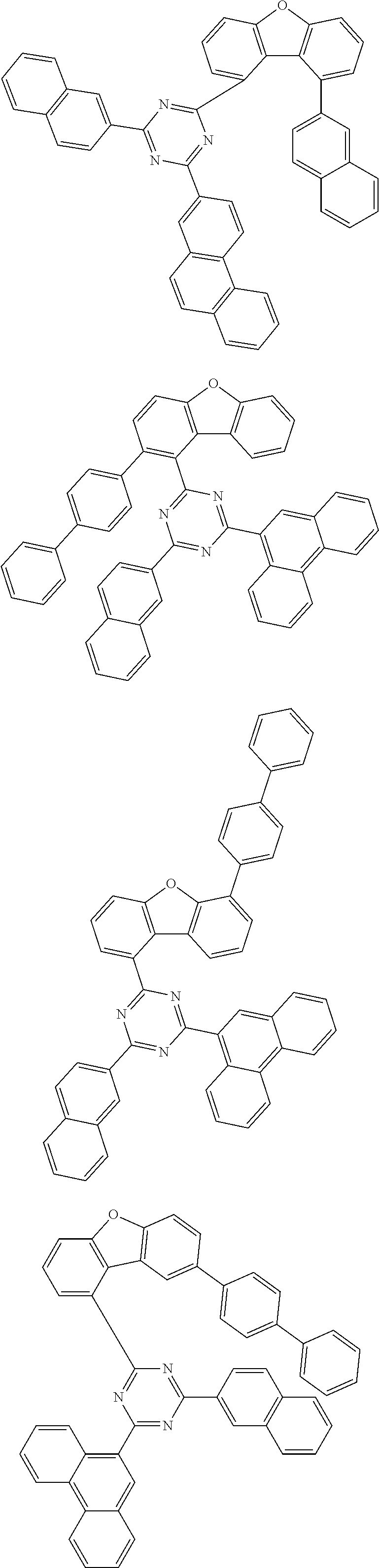

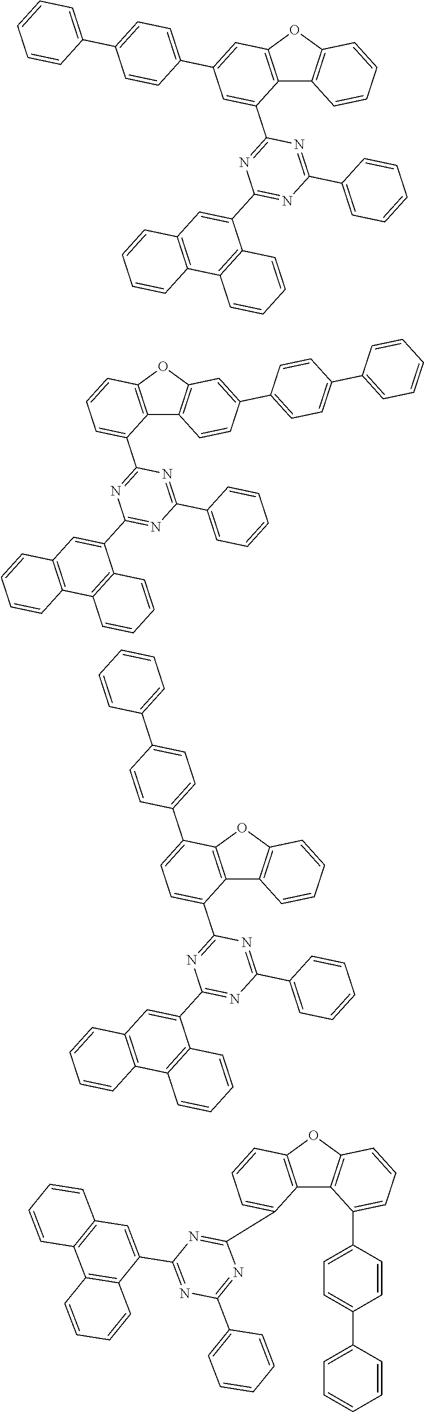

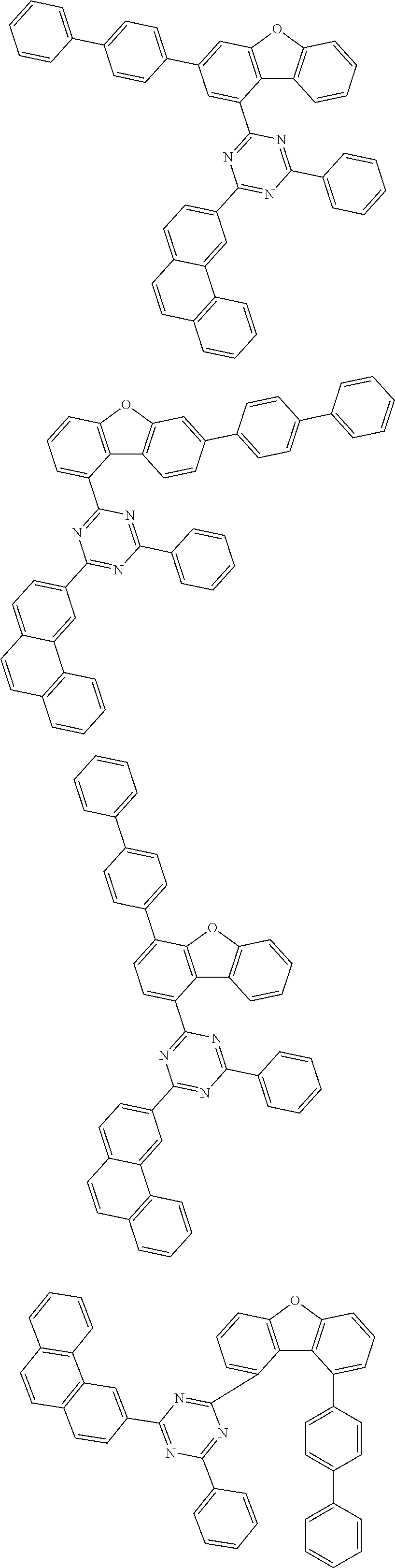

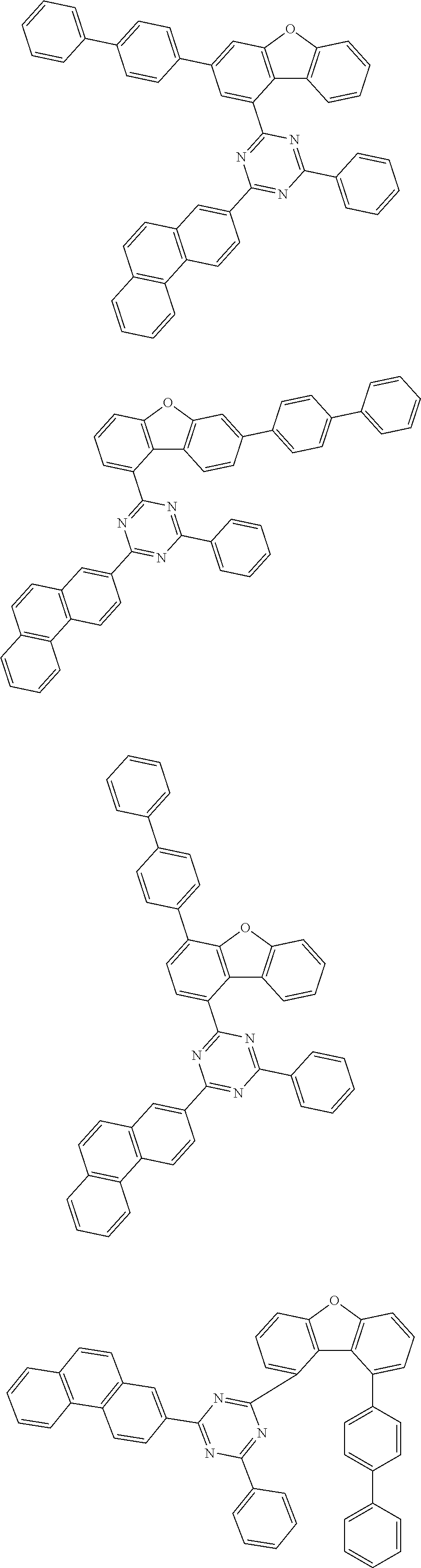 [SMILES: c1ccc(-c2ccc(-c3cc(-c4nc(-c5ccccc5)nc(-c5ccc6c(ccc7ccccc76)c5)n4)c4c(c3)oc3ccccc34)cc2)cc1 ; c1ccc(-c2ccc(-c3ccc4c(c3)oc3cccc(-c5nc(-c6ccccc6)nc(-c6ccc7c(ccc8ccccc87)c6)n5)c34)cc2)cc1 ; c1ccc(-c2ccc(-c3ccc(-c4nc(-c5ccccc5)nc(-c5ccc6c(ccc7ccccc76)c5)n4)c4c3oc3ccccc34)cc2)cc1 ; c1ccc(-c2ccc(-c3cccc4oc5cccc(-c6nc(-c7ccccc7)nc(-c7ccc8c(ccc9ccccc98)c7)n6)c5c34)cc2)cc1]

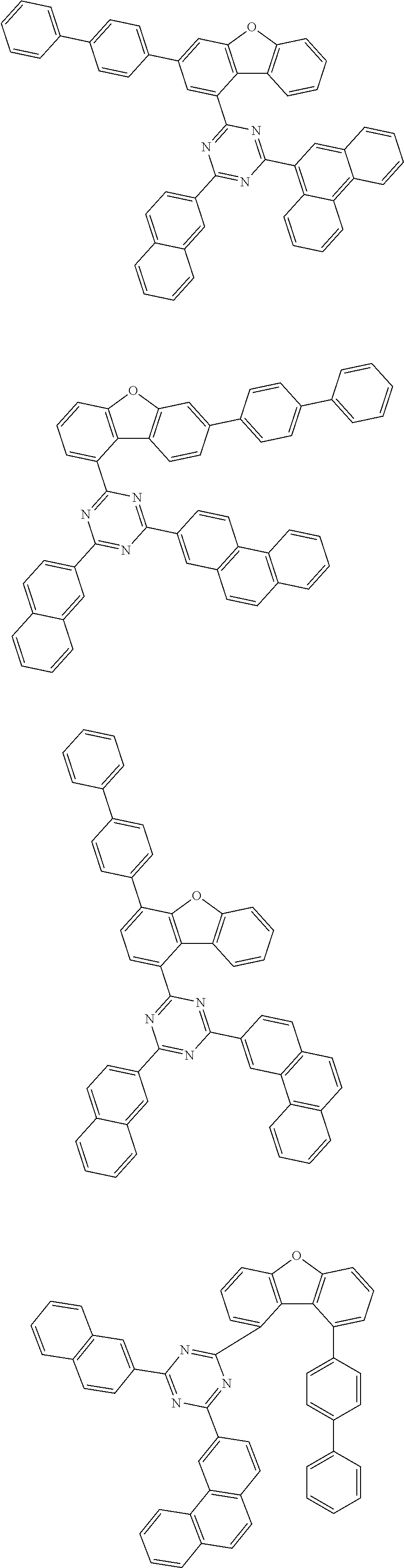 [SMILES: c1ccc(-c2ccc(-c3cc(-c4nc(-c5ccc6ccccc6c5)nc(-c5cc6ccccc6c6ccccc56)n4)c4c(c3)oc3ccccc34)cc2)cc1 ; c1ccc(-c2ccc(-c3ccc4c(c3)oc3cccc(-c5nc(-c6ccc7ccccc7c6)nc(-c6ccc7c(ccc8ccccc87)c6)n5)c34)cc2)cc1 ; c1ccc(-c2ccc(-c3ccc(-c4nc(-c5ccc6ccccc6c5)nc(-c5ccc6ccc7ccccc7c6c5)n4)c4c3oc3ccccc34)cc2)cc1 ; c1ccc(-c2ccc(-c3cccc4oc5cccc(-c6nc(-c7ccc8ccccc8c7)nc(-c7ccc8ccc9ccccc9c8c7)n6)c5c34)cc2)cc1]

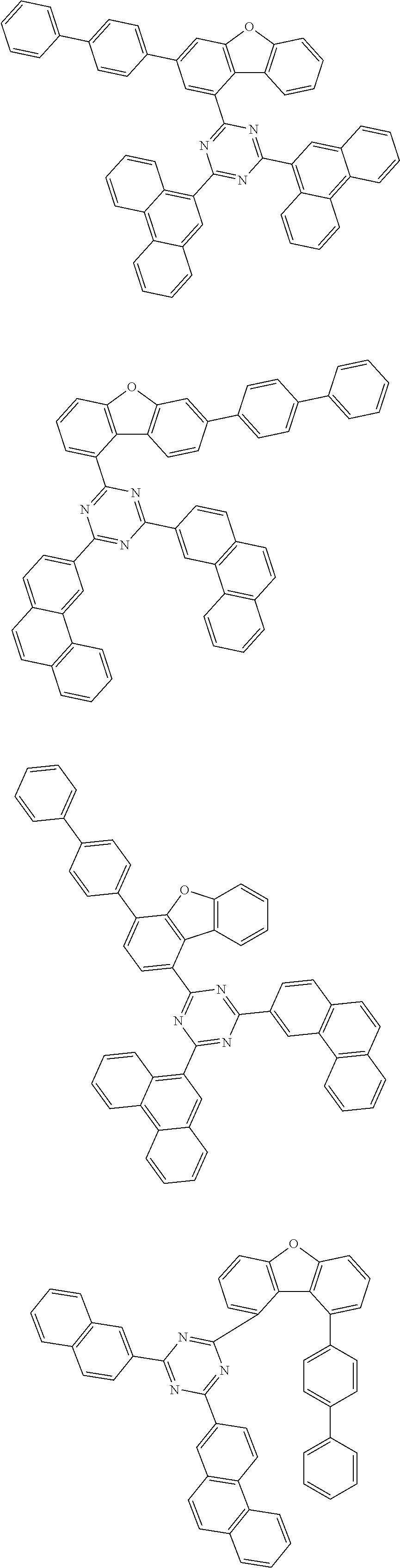 [SMILES: c1ccc(-c2ccc(-c3cc(-c4nc(-c5cc6ccccc6c6ccccc56)nc(-c5cc6ccccc6c6ccccc56)n4)c4c(c3)oc3ccccc34)cc2)cc1 ; c1ccc(-c2ccc(-c3ccc4c(c3)oc3cccc(-c5nc(-c6ccc7ccc8ccccc8c7c6)nc(-c6ccc7ccc8ccccc8c7c6)n5)c34)cc2)cc1 ; c1ccc(-c2ccc(-c3ccc(-c4nc(-c5ccc6ccc7ccccc7c6c5)nc(-c5cc6ccccc6c6ccccc56)n4)c4c3oc3ccccc34)cc2)cc1 ; c1ccc(-c2ccc(-c3cccc4oc5cccc(-c6nc(-c7ccc8ccccc8c7)nc(-c7ccc8c(ccc9ccccc98)c7)n6)c5c34)cc2)cc1]

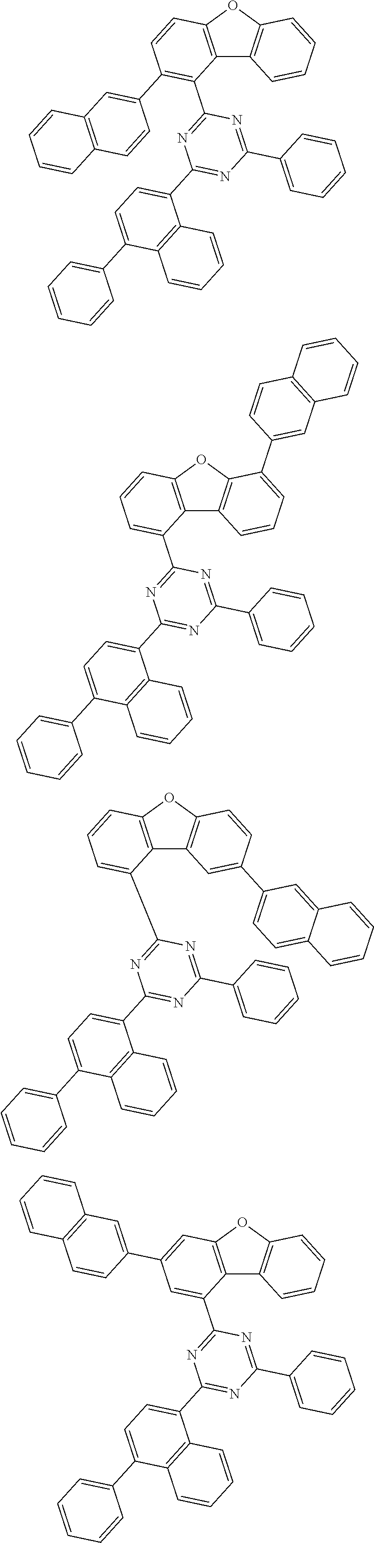

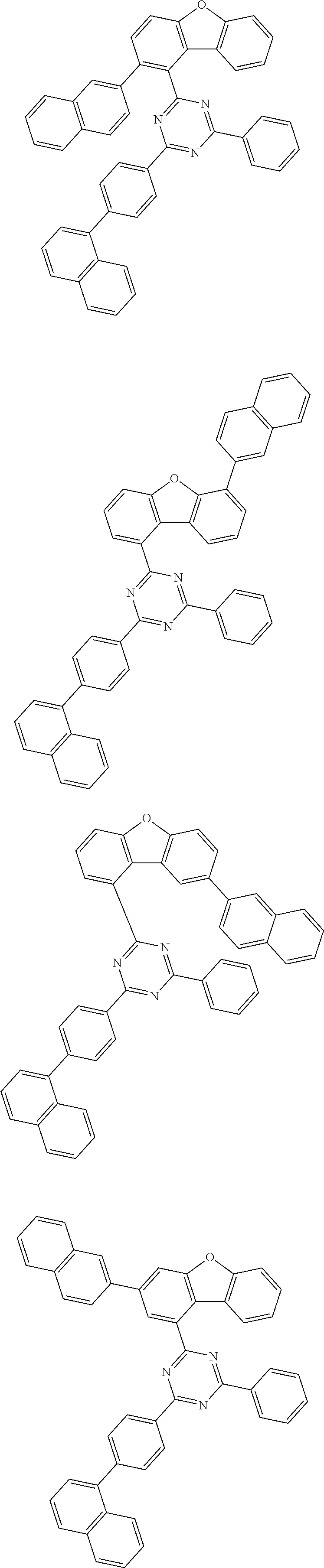 [SMILES: c1ccc(-c2nc(-c3ccc(-c4cccc5ccccc45)cc3)nc(-c3c(-c4ccc5ccccc5c4)ccc4oc5ccccc5c34)n2)cc1 ; c1ccc(-c2nc(-c3ccc(-c4cccc5ccccc45)cc3)nc(-c3cccc4oc5c(-c6ccc7ccccc7c6)cccc5c34)n2)cc1 ; c1ccc(-c2nc(-c3ccc(-c4cccc5ccccc45)cc3)nc(-c3cccc4oc5ccc(-c6ccc7ccccc7c6)cc5c34)n2)cc1 ; c1ccc(-c2nc(-c3ccc(-c4cccc5ccccc45)cc3)nc(-c3cc(-c4ccc5ccccc5c4)cc4oc5ccccc5c34)n2)cc1]

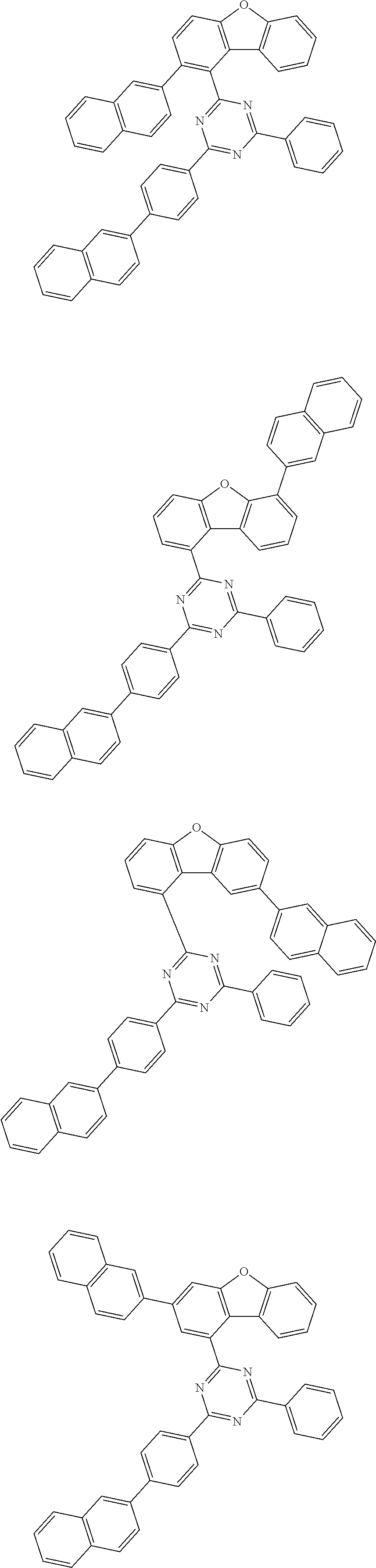 [SMILES: c1ccc(-c2nc(-c3ccc(-c4ccc5ccccc5c4)cc3)nc(-c3c(-c4ccc5ccccc5c4)ccc4oc5ccccc5c34)n2)cc1 ; c1ccc(-c2nc(-c3ccc(-c4ccc5ccccc5c4)cc3)nc(-c3cccc4oc5c(-c6ccc7ccccc7c6)cccc5c34)n2)cc1 ; c1ccc(-c2nc(-c3ccc(-c4ccc5ccccc5c4)cc3)nc(-c3cccc4oc5ccc(-c6ccc7ccccc7c6)cc5c34)n2)cc1 ; c1ccc(-c2nc(-c3ccc(-c4ccc5ccccc5c4)cc3)nc(-c3cc(-c4ccc5ccccc5c4)cc4oc5ccccc5c34)n2)cc1]

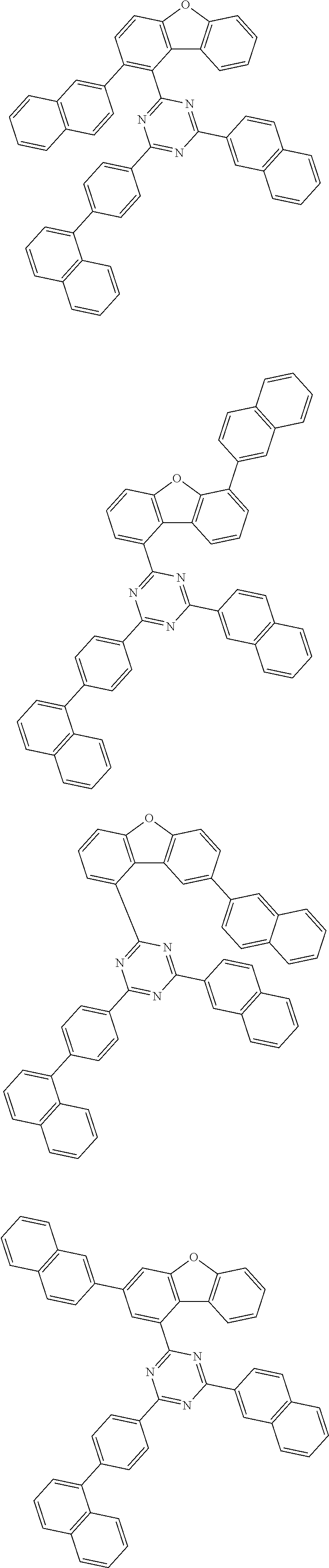 [SMILES: c1ccc2cc(-c3nc(-c4ccc(-c5cccc6ccccc56)cc4)nc(-c4c(-c5ccc6ccccc6c5)ccc5oc6ccccc6c45)n3)ccc2c1 ; c1ccc2cc(-c3nc(-c4ccc(-c5cccc6ccccc56)cc4)nc(-c4cccc5oc6c(-c7ccc8ccccc8c7)cccc6c45)n3)ccc2c1 ; c1ccc2cc(-c3ccc4oc5cccc(-c6nc(-c7ccc(-c8cccc9ccccc89)cc7)nc(-c7ccc8ccccc8c7)n6)c5c4c3)ccc2c1 ; c1ccc2cc(-c3cc(-c4nc(-c5ccc(-c6cccc7ccccc67)cc5)nc(-c5ccc6ccccc6c5)n4)c4c(c3)oc3ccccc34)ccc2c1]

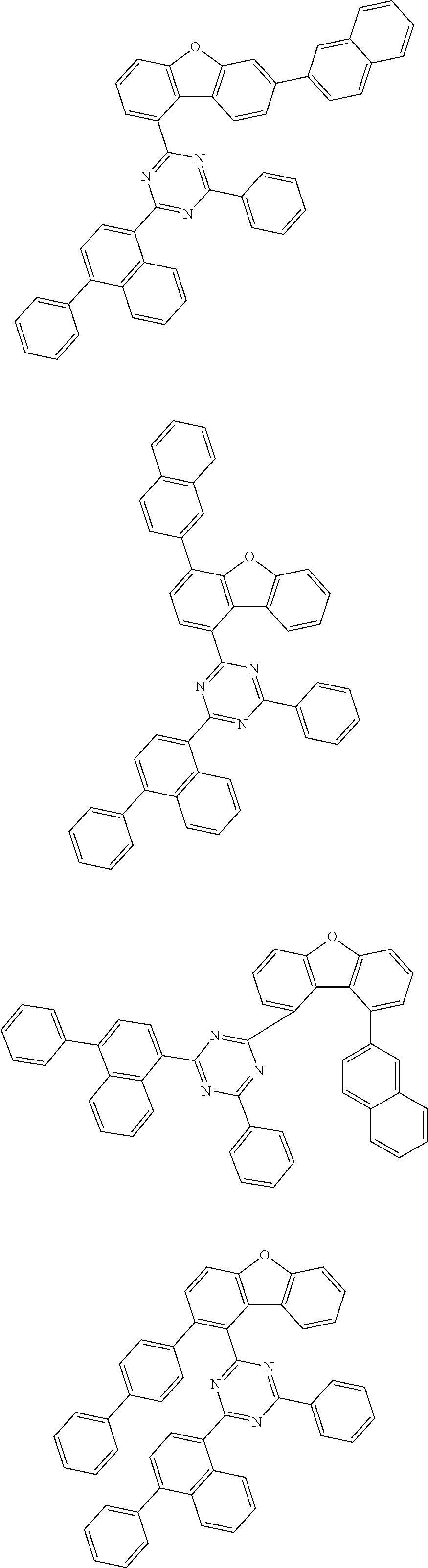 [SMILES: c1ccc(-c2nc(-c3ccc(-c4ccccc4)c4ccccc34)nc(-c3cccc4oc5cc(-c6ccc7ccccc7c6)ccc5c34)n2)cc1 ; c1ccc(-c2nc(-c3ccc(-c4ccccc4)c4ccccc34)nc(-c3ccc(-c4ccc5ccccc5c4)c4oc5ccccc5c34)n2)cc1 ; c1ccc(-c2nc(-c3ccc(-c4ccccc4)c4ccccc34)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccccc4)nc(-c4ccc(-c5ccccc5)c5ccccc45)n3)cc2)cc1]

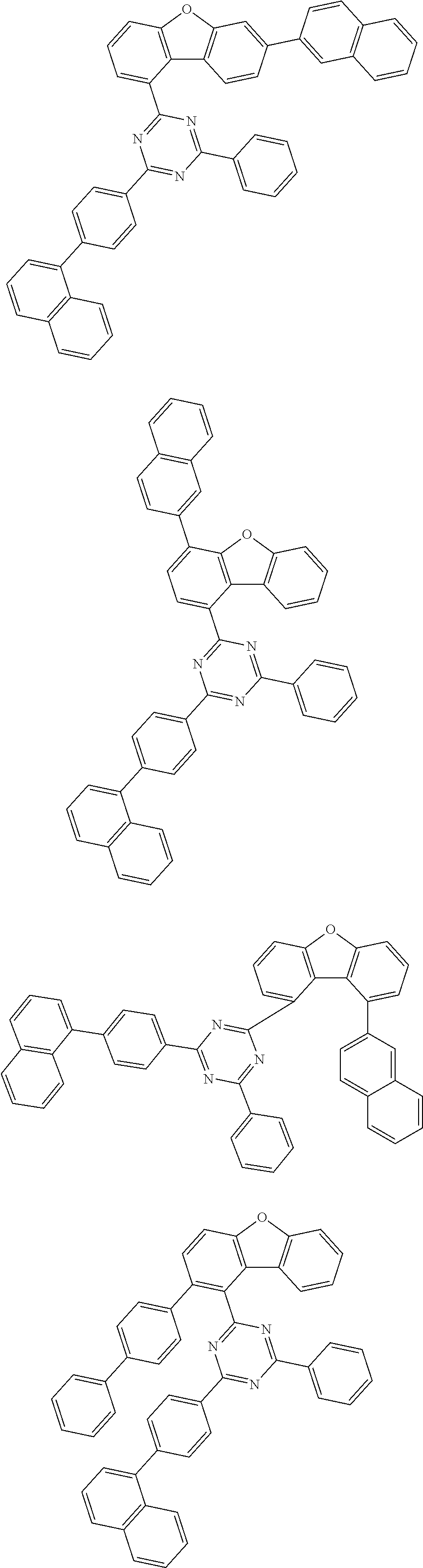 [SMILES: c1ccc(-c2nc(-c3ccc(-c4cccc5ccccc45)cc3)nc(-c3cccc4oc5cc(-c6ccc7ccccc7c6)ccc5c34)n2)cc1 ; c1ccc(-c2nc(-c3ccc(-c4cccc5ccccc45)cc3)nc(-c3ccc(-c4ccc5ccccc5c4)c4oc5ccccc5c34)n2)cc1 ; c1ccc(-c2nc(-c3ccc(-c4cccc5ccccc45)cc3)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccccc4)nc(-c4ccc(-c5cccc6ccccc56)cc4)n3)cc2)cc1]

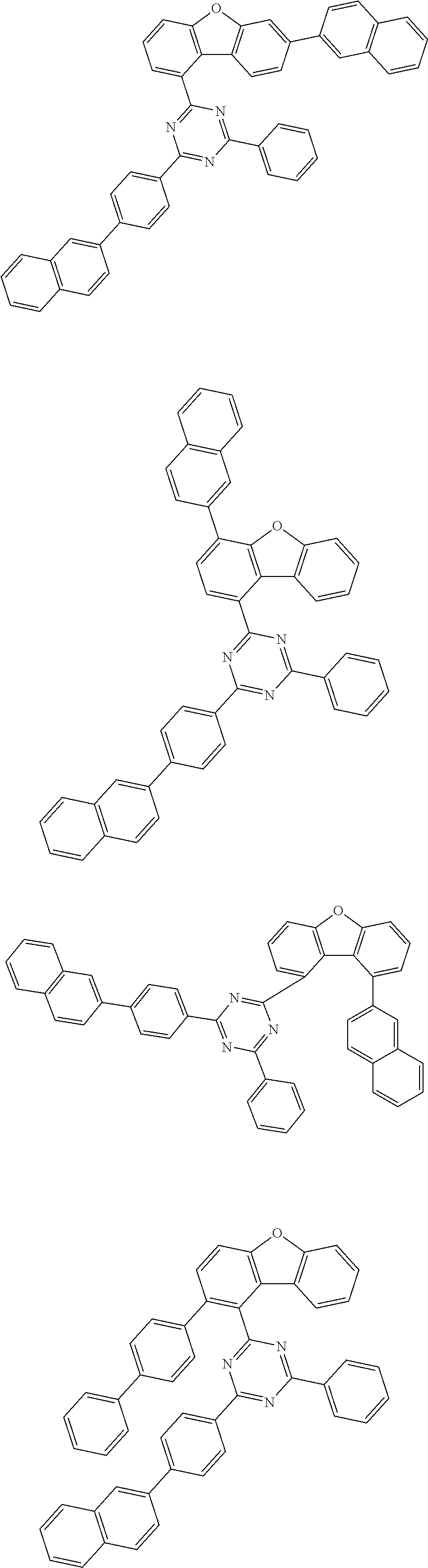 [SMILES: c1ccc(-c2nc(-c3ccc(-c4ccc5ccccc5c4)cc3)nc(-c3cccc4oc5cc(-c6ccc7ccccc7c6)ccc5c34)n2)cc1 ; c1ccc(-c2nc(-c3ccc(-c4ccc5ccccc5c4)cc3)nc(-c3ccc(-c4ccc5ccccc5c4)c4oc5ccccc5c34)n2)cc1 ; c1ccc(-c2nc(-c3ccc(-c4ccc5ccccc5c4)cc3)nc(-c3cccc4oc5cccc(-c6ccc7ccccc7c6)c5c34)n2)cc1 ; c1ccc(-c2ccc(-c3ccc4oc5ccccc5c4c3-c3nc(-c4ccccc4)nc(-c4ccc(-c5ccc6ccccc6c5)cc4)n3)cc2)cc1]

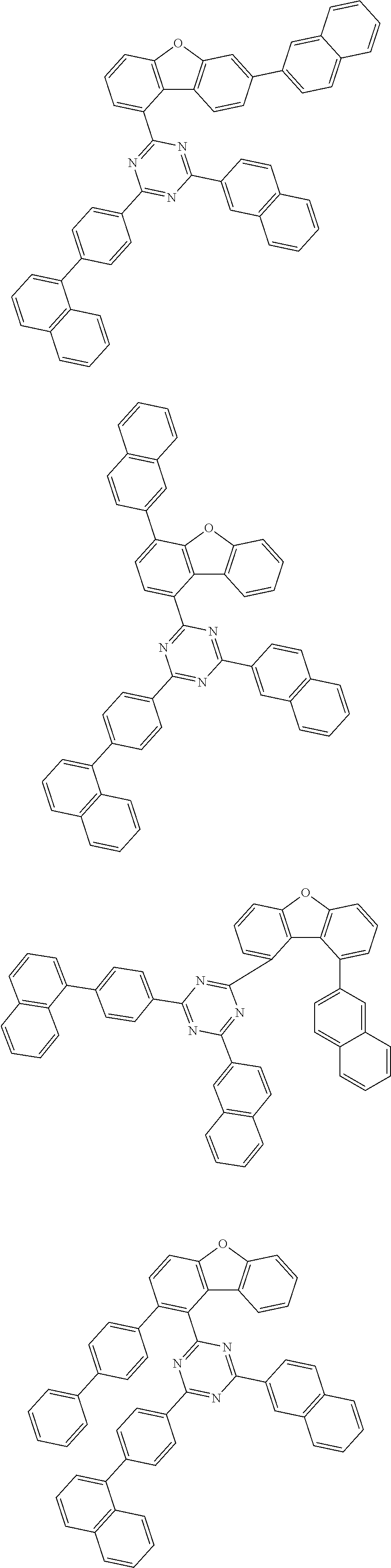 [SMILES: c1ccc2cc(-c3ccc4c(c3)oc3cccc(-c5nc(-c6ccc(-c7cccc8ccccc78)cc6)nc(-c6ccc7ccccc7c6)n5)c34)ccc2c1 ; c1ccc2cc(-c3nc(-c4ccc(-c5cccc6ccccc56)cc4)nc(-c4ccc(-c5ccc6ccccc6c5)c5oc6ccccc6c45)n3)ccc2c1 ; c1ccc2cc(-c3nc(-c4ccc(-c5cccc6ccccc56)cc4)nc(-c4cccc5oc6cccc(-c7ccc8ccccc8c7)c6c45)n3)ccc2c1 ; c1ccc(-c2ccc(-c3ccc4oc5ccccc5c4c3-c3nc(-c4ccc(-c5cccc6ccccc56)cc4)nc(-c4ccc5ccccc5c4)n3)cc2)cc1]

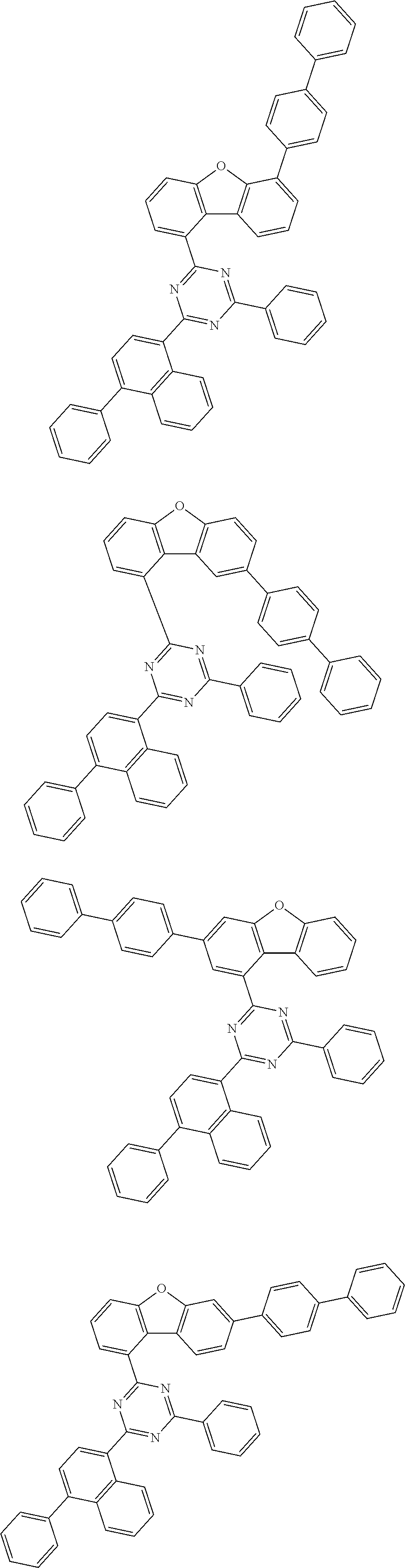 [SMILES: c1ccc(-c2ccc(-c3cccc4c3oc3cccc(-c5nc(-c6ccccc6)nc(-c6ccc(-c7ccccc7)c7ccccc67)n5)c34)cc2)cc1 ; c1ccc(-c2ccc(-c3ccc4oc5cccc(-c6nc(-c7ccccc7)nc(-c7ccc(-c8ccccc8)c8ccccc78)n6)c5c4c3)cc2)cc1 ; c1ccc(-c2ccc(-c3cc(-c4nc(-c5ccccc5)nc(-c5ccc(-c6ccccc6)c6ccccc56)n4)c4c(c3)oc3ccccc34)cc2)cc1 ; c1ccc(-c2ccc(-c3ccc4c(c3)oc3cccc(-c5nc(-c6ccccc6)nc(-c6ccc(-c7ccccc7)c7ccccc67)n5)c34)cc2)cc1]

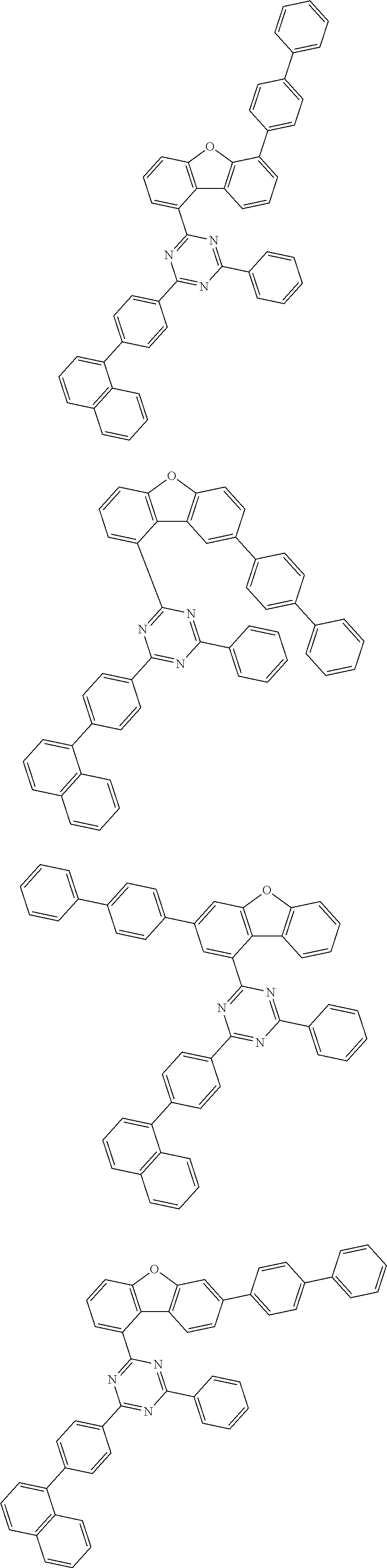 [SMILES: c1ccc(-c2ccc(-c3cccc4c3oc3cccc(-c5nc(-c6ccccc6)nc(-c6ccc(-c7cccc8ccccc78)cc6)n5)c34)cc2)cc1 ; c1ccc(-c2ccc(-c3ccc4oc5cccc(-c6nc(-c7ccccc7)nc(-c7ccc(-c8cccc9ccccc89)cc7)n6)c5c4c3)cc2)cc1 ; c1ccc(-c2ccc(-c3cc(-c4nc(-c5ccccc5)nc(-c5ccc(-c6cccc7ccccc67)cc5)n4)c4c(c3)oc3ccccc34)cc2)cc1 ; c1ccc(-c2ccc(-c3ccc4c(c3)oc3cccc(-c5nc(-c6ccccc6)nc(-c6ccc(-c7cccc8ccccc78)cc6)n5)c34)cc2)cc1]

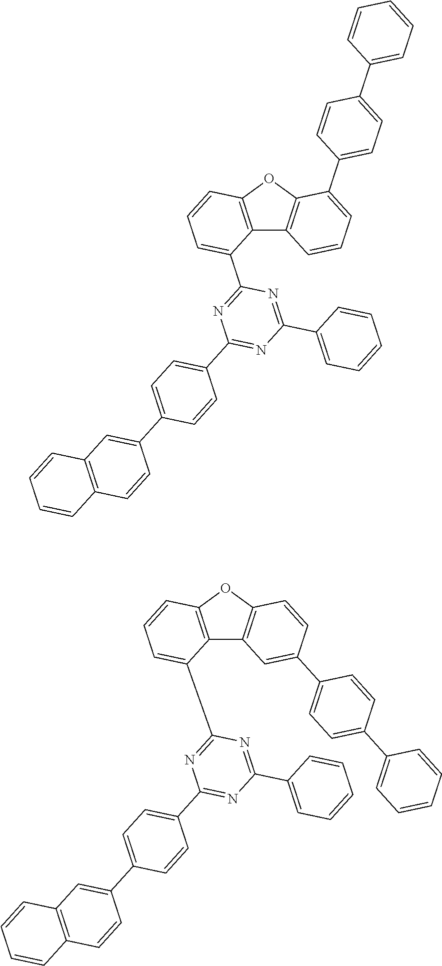 [SMILES: c1ccc(-c2ccc(-c3cccc4c3oc3cccc(-c5nc(-c6ccccc6)nc(-c6ccc(-c7ccc8ccccc8c7)cc6)n5)c34)cc2)cc1 ; c1ccc(-c2ccc(-c3ccc4oc5cccc(-c6nc(-c7ccccc7)nc(-c7ccc(-c8ccc9ccccc9c8)cc7)n6)c5c4c3)cc2)cc1]

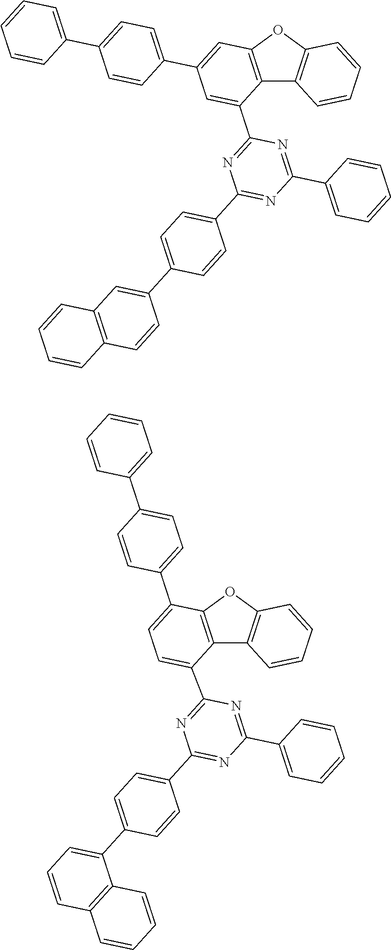 [SMILES: c1ccc(-c2ccc(-c3cc(-c4nc(-c5ccccc5)nc(-c5ccc(-c6ccc7ccccc7c6)cc5)n4)c4c(c3)oc3ccccc34)cc2)cc1 ; c1ccc(-c2ccc(-c3ccc(-c4nc(-c5ccccc5)nc(-c5ccc(-c6cccc7ccccc67)cc5)n4)c4c3oc3ccccc34)cc2)cc1]

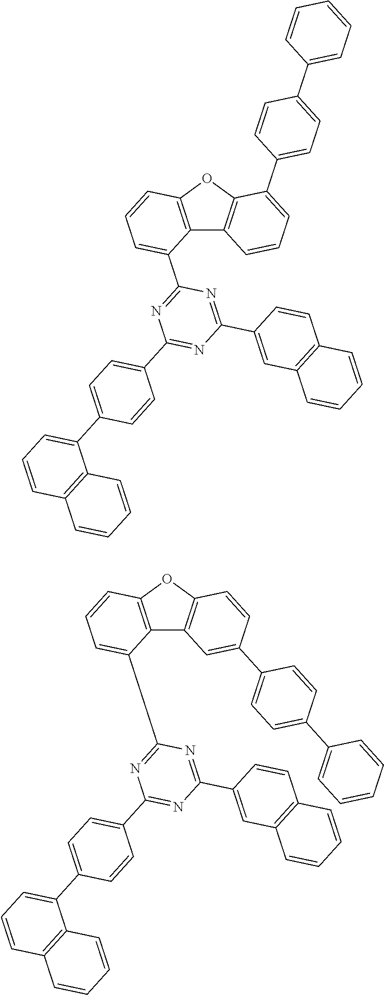 [SMILES: c1ccc(-c2ccc(-c3cccc4c3oc3cccc(-c5nc(-c6ccc(-c7cccc8ccccc78)cc6)nc(-c6ccc7ccccc7c6)n5)c34)cc2)cc1 ; c1ccc(-c2ccc(-c3ccc4oc5cccc(-c6nc(-c7ccc(-c8cccc9ccccc89)cc7)nc(-c7ccc8ccccc8c7)n6)c5c4c3)cc2)cc1]

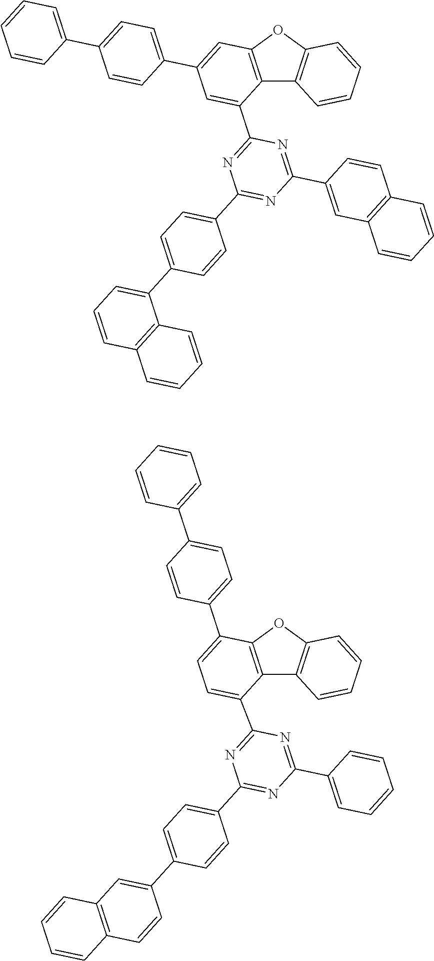 [SMILES: c1ccc(-c2ccc(-c3cc(-c4nc(-c5ccc(-c6cccc7ccccc67)cc5)nc(-c5ccc6ccccc6c5)n4)c4c(c3)oc3ccccc34)cc2)cc1 ; c1ccc(-c2ccc(-c3ccc(-c4nc(-c5ccccc5)nc(-c5ccc(-c6ccc7ccccc7c6)cc5)n4)c4c3oc3ccccc34)cc2)cc1]

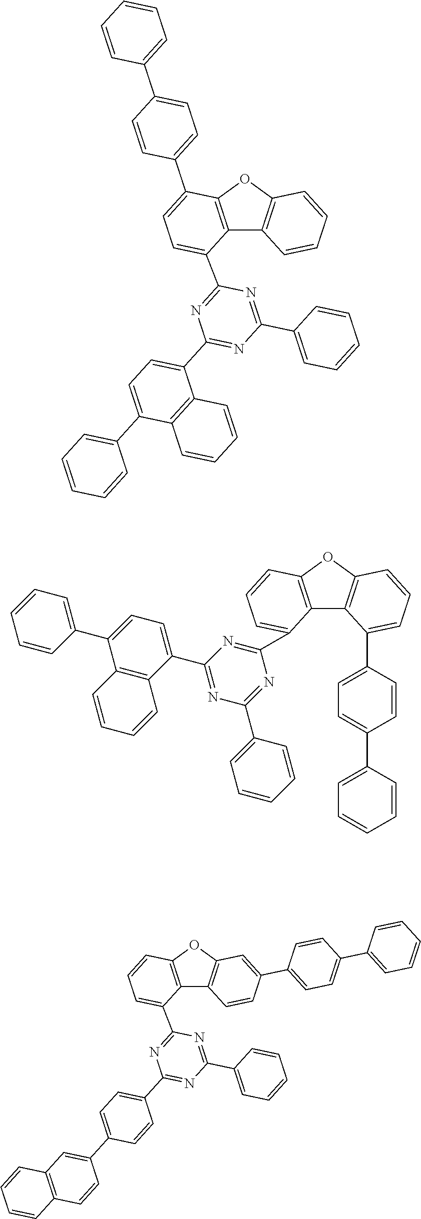 [SMILES: c1ccc(-c2ccc(-c3ccc(-c4nc(-c5ccccc5)nc(-c5ccc(-c6ccccc6)c6ccccc56)n4)c4c3oc3ccccc34)cc2)cc1 ; c1ccc(-c2ccc(-c3cccc4oc5cccc(-c6nc(-c7ccccc7)nc(-c7ccc(-c8ccccc8)c8ccccc78)n6)c5c34)cc2)cc1 ; c1ccc(-c2ccc(-c3ccc4c(c3)oc3cccc(-c5nc(-c6ccccc6)nc(-c6ccc(-c7ccc8ccccc8c7)cc6)n5)c34)cc2)cc1]

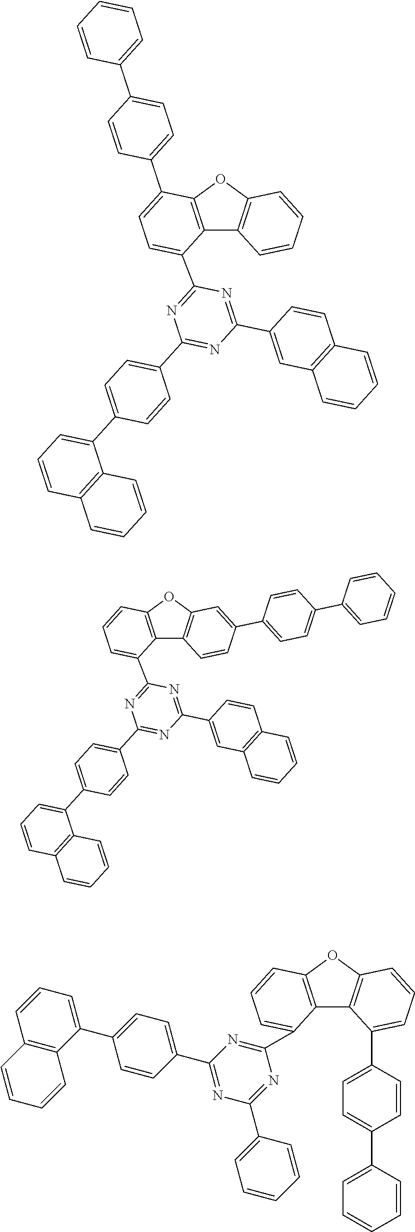 [SMILES: c1ccc(-c2ccc(-c3ccc(-c4nc(-c5ccc(-c6cccc7ccccc67)cc5)nc(-c5ccc6ccccc6c5)n4)c4c3oc3ccccc34)cc2)cc1 ; c1ccc(-c2ccc(-c3ccc4c(c3)oc3cccc(-c5nc(-c6ccc(-c7cccc8ccccc78)cc6)nc(-c6ccc7ccccc7c6)n5)c34)cc2)cc1 ; c1ccc(-c2ccc(-c3cccc4oc5cccc(-c6nc(-c7ccccc7)nc(-c7ccc(-c8cccc9ccccc89)cc7)n6)c5c34)cc2)cc1]

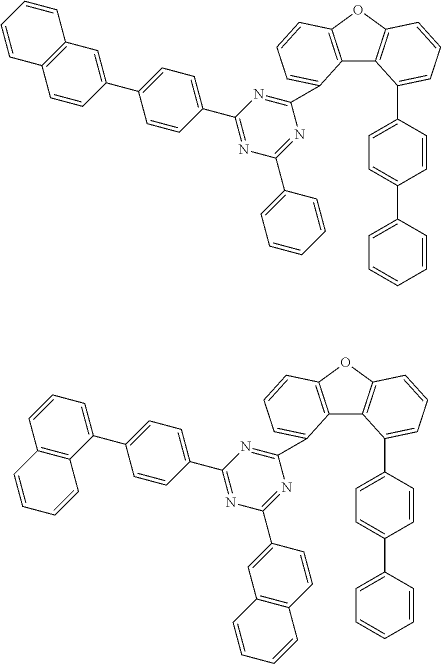 [SMILES: c1ccc(-c2ccc(-c3cccc4oc5cccc(-c6nc(-c7ccccc7)nc(-c7ccc(-c8ccc9ccccc9c8)cc7)n6)c5c34)cc2)cc1 ; c1ccc(-c2ccc(-c3cccc4oc5cccc(-c6nc(-c7ccc(-c8cccc9ccccc89)cc7)nc(-c7ccc8ccccc8c7)n6)c5c34)cc2)cc1]

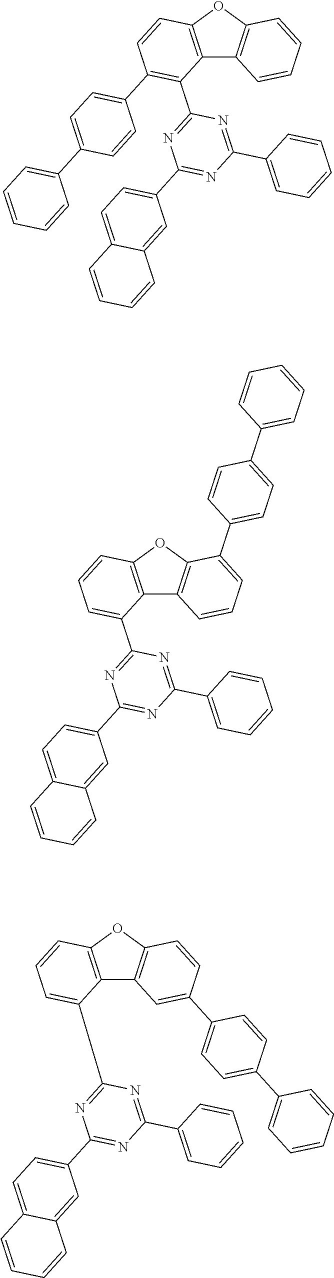 [SMILES: c1ccc(-c2ccc(-c3ccc4oc5ccccc5c4c3-c3nc(-c4ccccc4)nc(-c4ccc5ccccc5c4)n3)cc2)cc1 ; c1ccc(-c2ccc(-c3cccc4c3oc3cccc(-c5nc(-c6ccccc6)nc(-c6ccc7ccccc7c6)n5)c34)cc2)cc1 ; c1ccc(-c2ccc(-c3ccc4oc5cccc(-c6nc(-c7ccccc7)nc(-c7ccc8ccccc8c7)n6)c5c4c3)cc2)cc1]

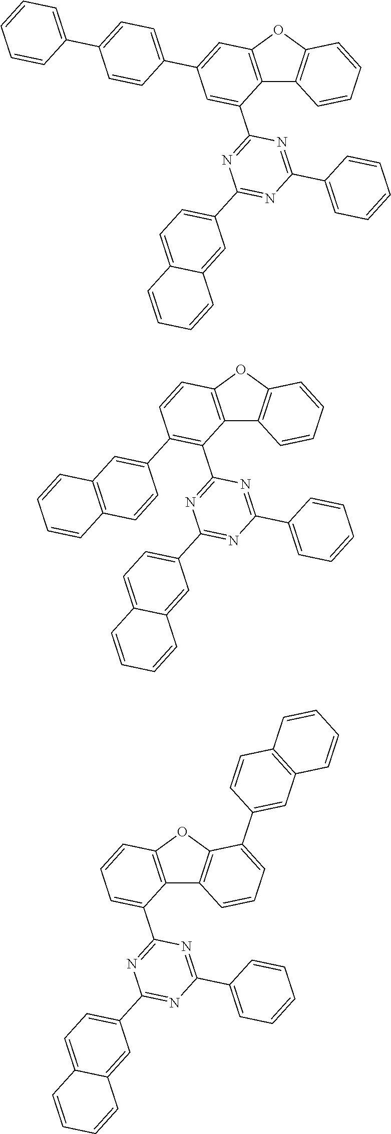 [SMILES: c1ccc(-c2ccc(-c3cc(-c4nc(-c5ccccc5)nc(-c5ccc6ccccc6c5)n4)c4c(c3)oc3ccccc34)cc2)cc1 ; c1ccc(-c2nc(-c3ccc4ccccc4c3)nc(-c3c(-c4ccc5ccccc5c4)ccc4oc5ccccc5c34)n2)cc1 ; c1ccc(-c2nc(-c3ccc4ccccc4c3)nc(-c3cccc4oc5c(-c6ccc7ccccc7c6)cccc5c34)n2)cc1]

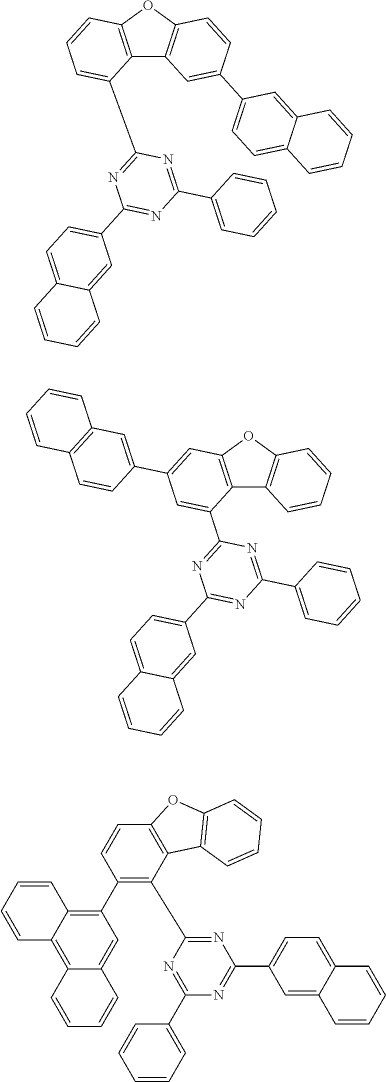 [SMILES: c1ccc(-c2nc(-c3ccc4ccccc4c3)nc(-c3cccc4oc5ccc(-c6ccc7ccccc7c6)cc5c34)n2)cc1 ; c1ccc(-c2nc(-c3ccc4ccccc4c3)nc(-c3cc(-c4ccc5ccccc5c4)cc4oc5ccccc5c34)n2)cc1 ; c1ccc(-c2nc(-c3ccc4ccccc4c3)nc(-c3c(-c4cc5ccccc5c5ccccc45)ccc4oc5ccccc5c34)n2)cc1]

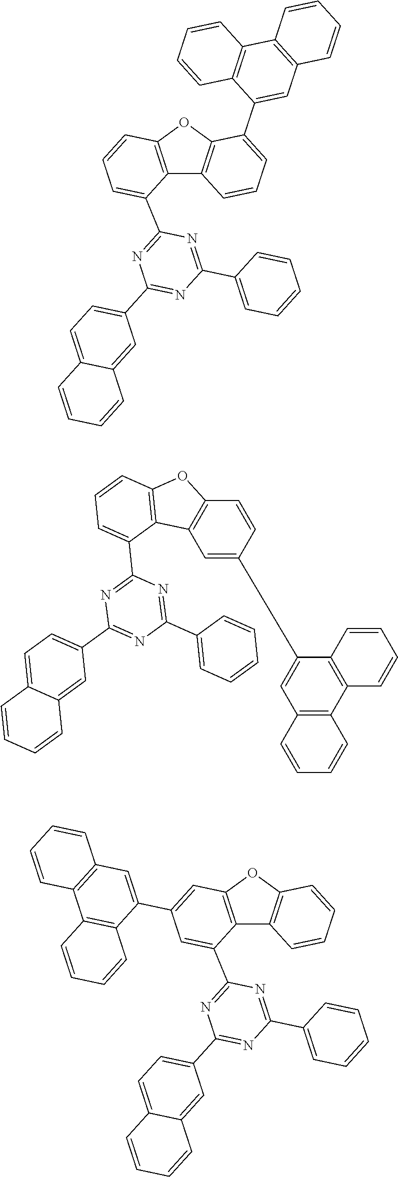 [SMILES: c1ccc(-c2nc(-c3ccc4ccccc4c3)nc(-c3cccc4oc5c(-c6cc7ccccc7c7ccccc67)cccc5c34)n2)cc1 ; c1ccc(-c2nc(-c3ccc4ccccc4c3)nc(-c3cccc4oc5ccc(-c6cc7ccccc7c7ccccc67)cc5c34)n2)cc1 ; c1ccc(-c2nc(-c3ccc4ccccc4c3)nc(-c3cc(-c4cc5ccccc5c5ccccc45)cc4oc5ccccc5c34)n2)cc1]

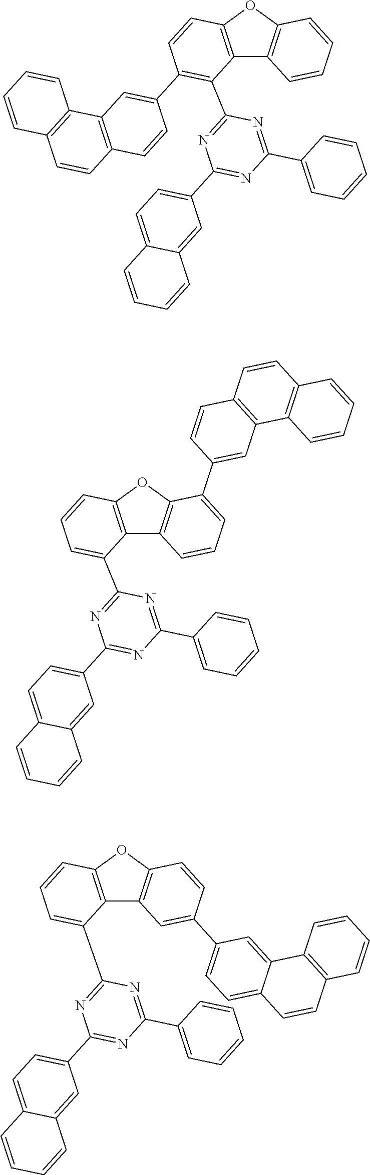 [SMILES: c1ccc(-c2nc(-c3ccc4ccccc4c3)nc(-c3c(-c4ccc5ccc6ccccc6c5c4)ccc4oc5ccccc5c34)n2)cc1 ; c1ccc(-c2nc(-c3ccc4ccccc4c3)nc(-c3cccc4oc5c(-c6ccc7ccc8ccccc8c7c6)cccc5c34)n2)cc1 ; c1ccc(-c2nc(-c3ccc4ccccc4c3)nc(-c3cccc4oc5ccc(-c6ccc7ccc8ccccc8c7c6)cc5c34)n2)cc1]

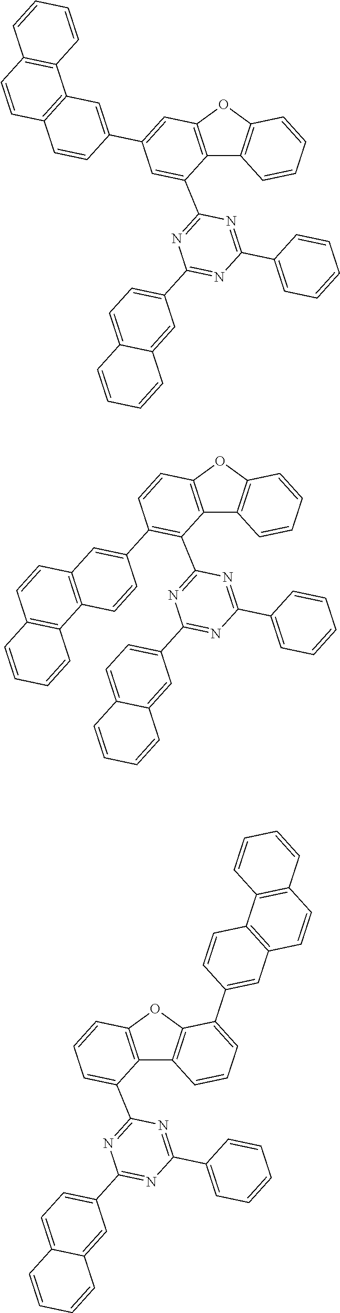 [SMILES: c1ccc(-c2nc(-c3ccc4ccccc4c3)nc(-c3cc(-c4ccc5ccc6ccccc6c5c4)cc4oc5ccccc5c34)n2)cc1 ; c1ccc(-c2nc(-c3ccc4ccccc4c3)nc(-c3c(-c4ccc5c(ccc6ccccc65)c4)ccc4oc5ccccc5c34)n2)cc1 ; c1ccc(-c2nc(-c3ccc4ccccc4c3)nc(-c3cccc4oc5c(-c6ccc7c(ccc8ccccc87)c6)cccc5c34)n2)cc1]

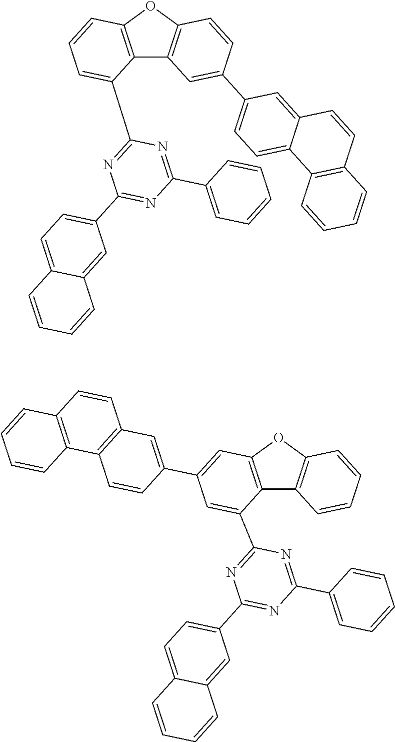 [SMILES: c1ccc(-c2nc(-c3ccc4ccccc4c3)nc(-c3cccc4oc5ccc(-c6ccc7c(ccc8ccccc87)c6)cc5c34)n2)cc1 ; c1ccc(-c2nc(-c3ccc4ccccc4c3)nc(-c3cc(-c4ccc5c(ccc6ccccc65)c4)cc4oc5ccccc5c34)n2)cc1]

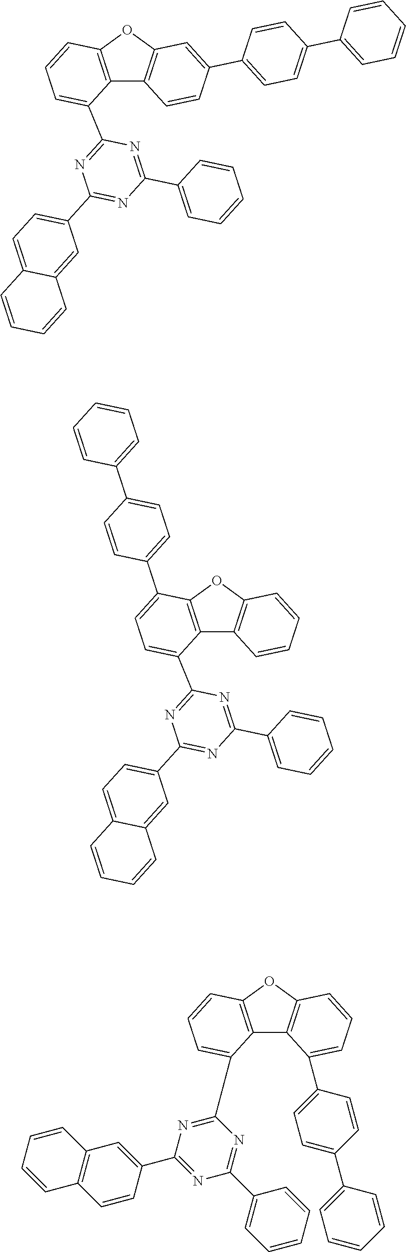 [SMILES: c1ccc(-c2ccc(-c3ccc4c(c3)oc3cccc(-c5nc(-c6ccccc6)nc(-c6ccc7ccccc7c6)n5)c34)cc2)cc1 ; c1ccc(-c2ccc(-c3ccc(-c4nc(-c5ccccc5)nc(-c5ccc6ccccc6c5)n4)c4c3oc3ccccc34)cc2)cc1 ; c1ccc(-c2ccc(-c3cccc4oc5cccc(-c6nc(-c7ccccc7)nc(-c7ccc8ccccc8c7)n6)c5c34)cc2)cc1]

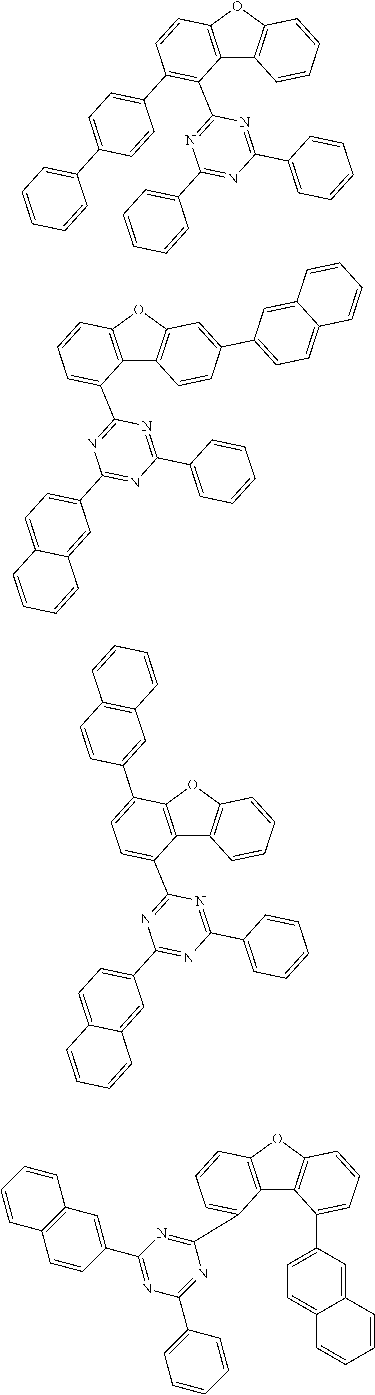 [SMILES: c1ccc(-c2ccc(-c3ccc4oc5ccccc5c4c3-c3nc(-c4ccccc4)nc(-c4ccccc4)n3)cc2)cc1 ; c1ccc(-c2nc(-c3ccc4ccccc4c3)nc(-c3cccc4oc5cc(-c6ccc7ccccc7c6)ccc5c34)n2)cc1 ; c1ccc(-c2nc(-c3ccc4ccccc4c3)nc(-c3ccc(-c4ccc5ccccc5c4)c4oc5ccccc5c34)n2)cc1 ; c1ccc(-c2nc(-c3ccc4ccccc4c3)nc(-c3cccc4oc5cccc(-c6ccc7ccccc7c6)c5c34)n2)cc1]

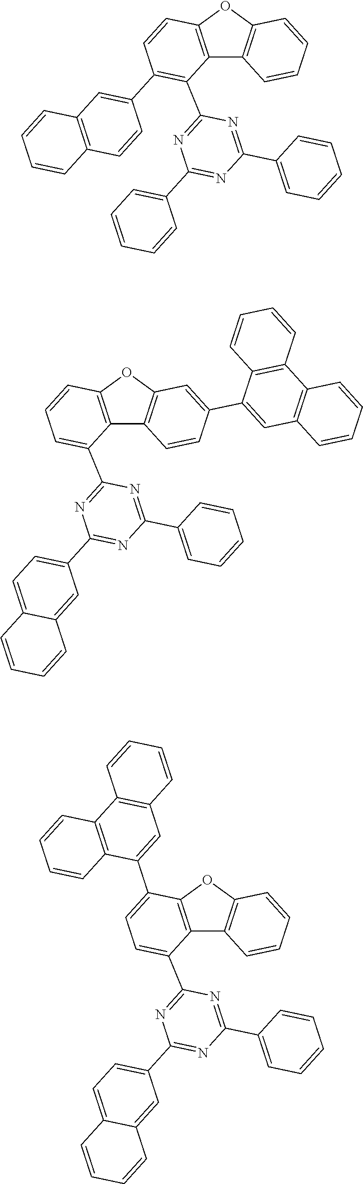 [SMILES: c1ccc(-c2nc(-c3ccccc3)nc(-c3c(-c4ccc5ccccc5c4)ccc4oc5ccccc5c34)n2)cc1 ; c1ccc(-c2nc(-c3ccc4ccccc4c3)nc(-c3cccc4oc5cc(-c6cc7ccccc7c7ccccc67)ccc5c34)n2)cc1 ; c1ccc(-c2nc(-c3ccc4ccccc4c3)nc(-c3ccc(-c4cc5ccccc5c5ccccc45)c4oc5ccccc5c34)n2)cc1]

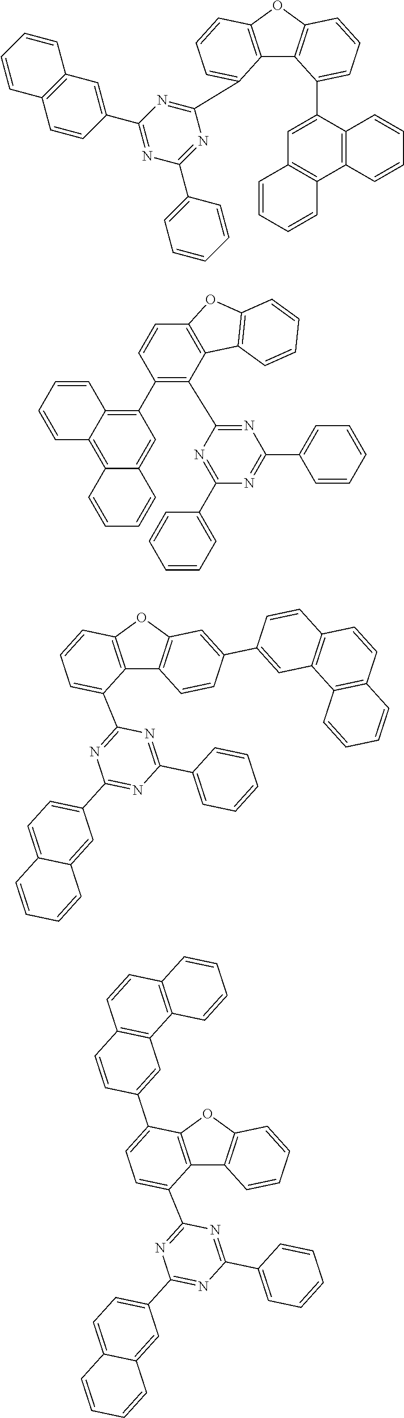

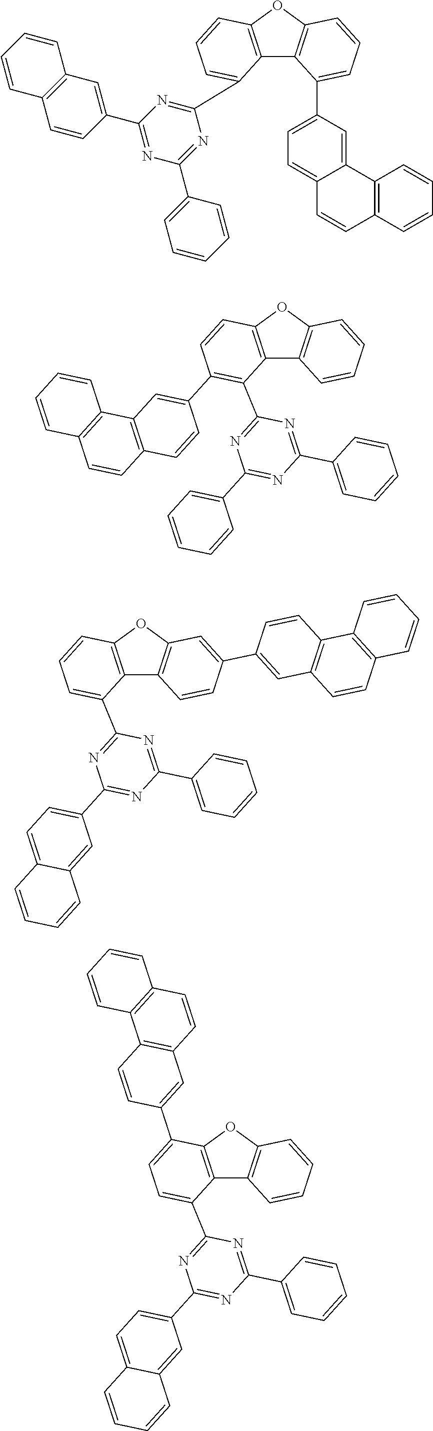 [SMILES: c1ccc(-c2nc(-c3ccc4ccccc4c3)nc(-c3cccc4oc5cccc(-c6ccc7ccc8ccccc8c7c6)c5c34)n2)cc1 ; c1ccc(-c2nc(-c3ccccc3)nc(-c3c(-c4ccc5ccc6ccccc6c5c4)ccc4oc5ccccc5c34)n2)cc1 ; c1ccc(-c2nc(-c3ccc4ccccc4c3)nc(-c3cccc4oc5cc(-c6ccc7c(ccc8ccccc87)c6)ccc5c34)n2)cc1 ; c1ccc(-c2nc(-c3ccc4ccccc4c3)nc(-c3ccc(-c4ccc5c(ccc6ccccc65)c4)c4oc5ccccc5c34)n2)cc1]

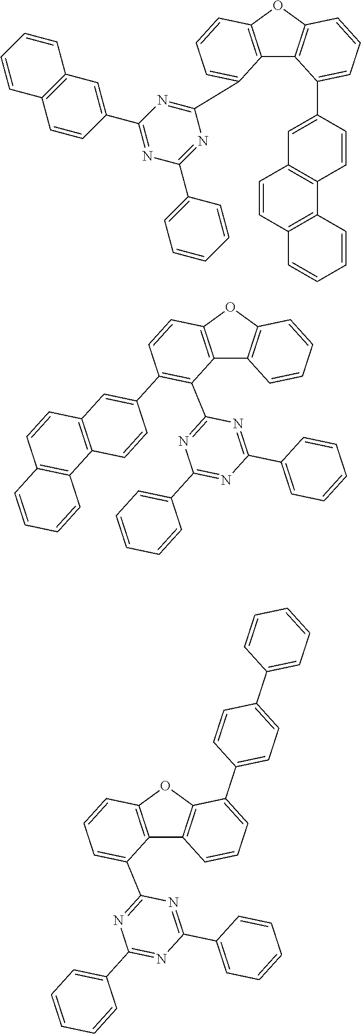

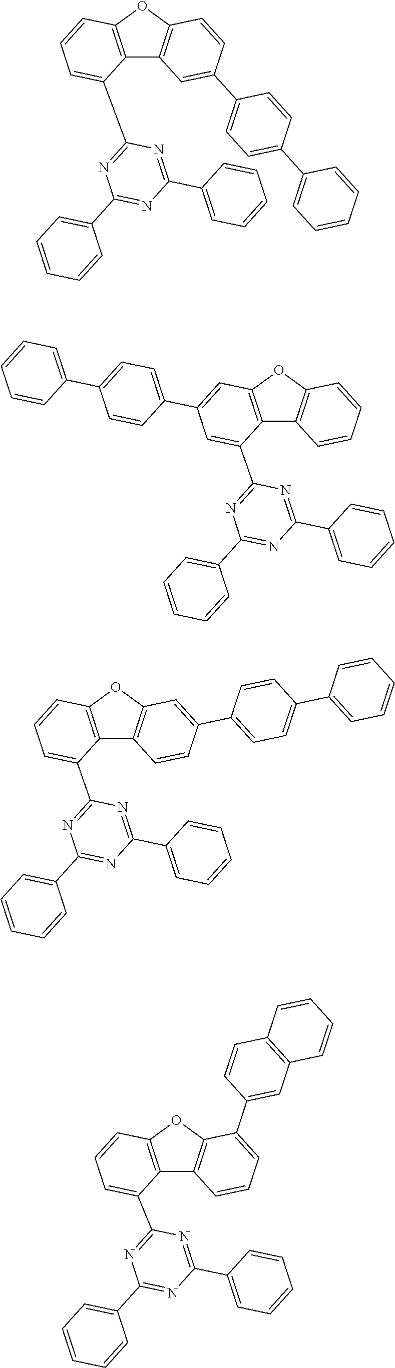 [SMILES: c1ccc(-c2ccc(-c3ccc4oc5cccc(-c6nc(-c7ccccc7)nc(-c7ccccc7)n6)c5c4c3)cc2)cc1 ; c1ccc(-c2ccc(-c3cc(-c4nc(-c5ccccc5)nc(-c5ccccc5)n4)c4c(c3)oc3ccccc34)cc2)cc1 ; c1ccc(-c2ccc(-c3ccc4c(c3)oc3cccc(-c5nc(-c6ccccc6)nc(-c6ccccc6)n5)c34)cc2)cc1 ; c1ccc(-c2nc(-c3ccccc3)nc(-c3cccc4oc5c(-c6ccc7ccccc7c6)cccc5c34)n2)cc1]

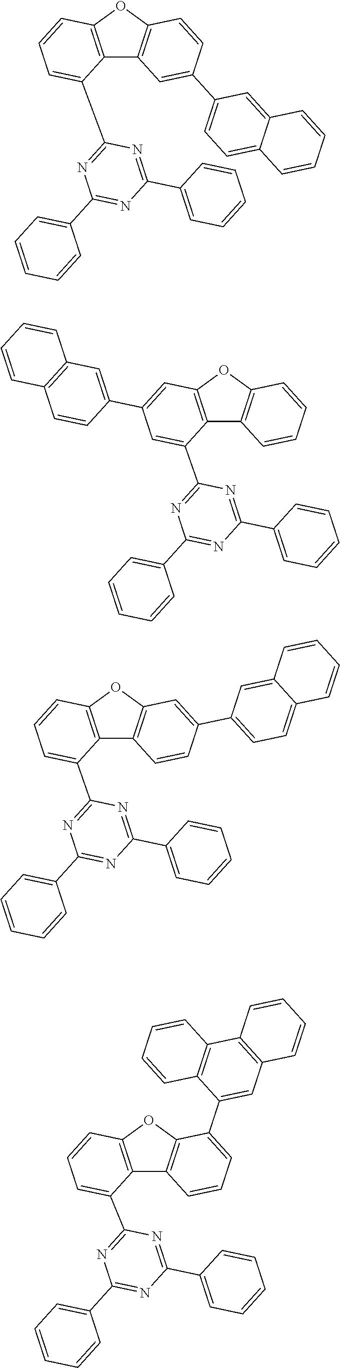 [SMILES: c1ccc(-c2nc(-c3ccccc3)nc(-c3cccc4oc5ccc(-c6ccc7ccccc7c6)cc5c34)n2)cc1 ; c1ccc(-c2nc(-c3ccccc3)nc(-c3cc(-c4ccc5ccccc5c4)cc4oc5ccccc5c34)n2)cc1 ; c1ccc(-c2nc(-c3ccccc3)nc(-c3cccc4oc5cc(-c6ccc7ccccc7c6)ccc5c34)n2)cc1 ; c1ccc(-c2nc(-c3ccccc3)nc(-c3cccc4oc5c(-c6cc7ccccc7c7ccccc67)cccc5c34)n2)cc1]

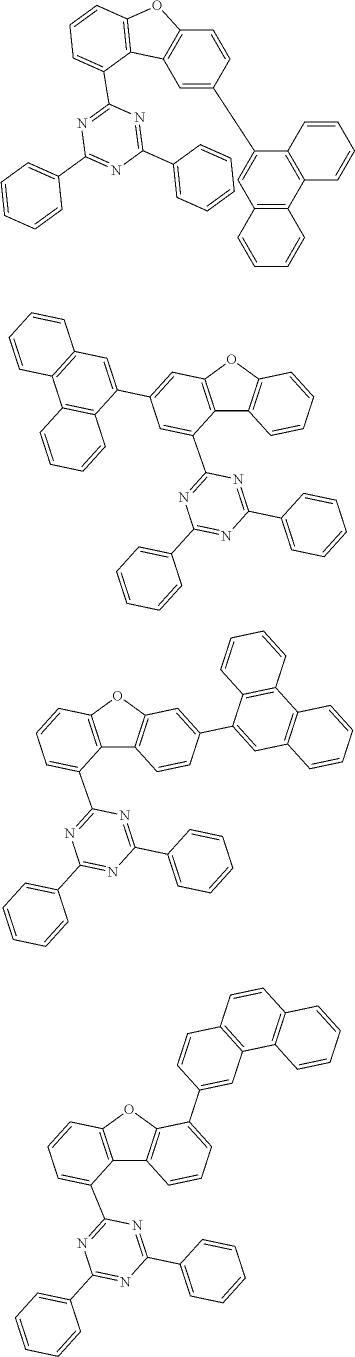 [SMILES: c1ccc(-c2nc(-c3ccccc3)nc(-c3cccc4oc5ccc(-c6cc7ccccc7c7ccccc67)cc5c34)n2)cc1 ; c1ccc(-c2nc(-c3ccccc3)nc(-c3cc(-c4cc5ccccc5c5ccccc45)cc4oc5ccccc5c34)n2)cc1 ; c1ccc(-c2nc(-c3ccccc3)nc(-c3cccc4oc5cc(-c6cc7ccccc7c7ccccc67)ccc5c34)n2)cc1 ; c1ccc(-c2nc(-c3ccccc3)nc(-c3cccc4oc5c(-c6ccc7ccc8ccccc8c7c6)cccc5c34)n2)cc1]

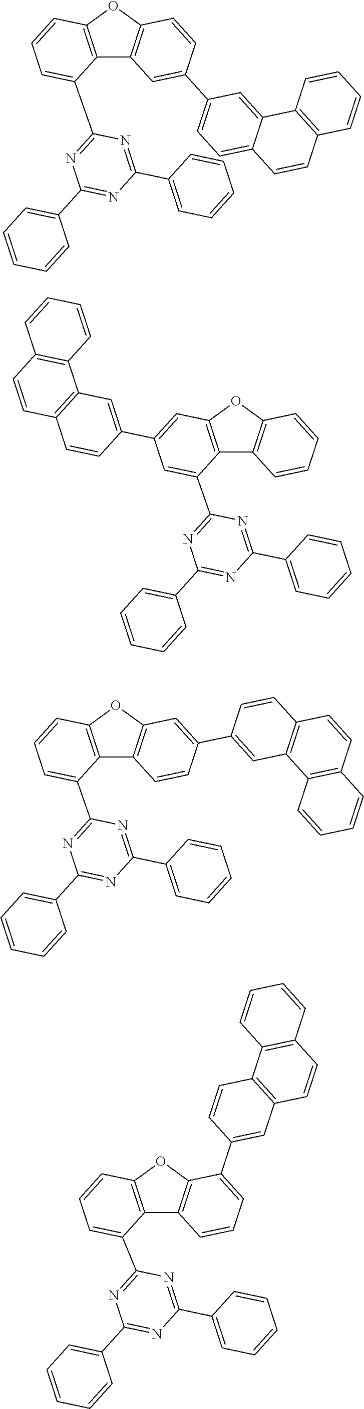

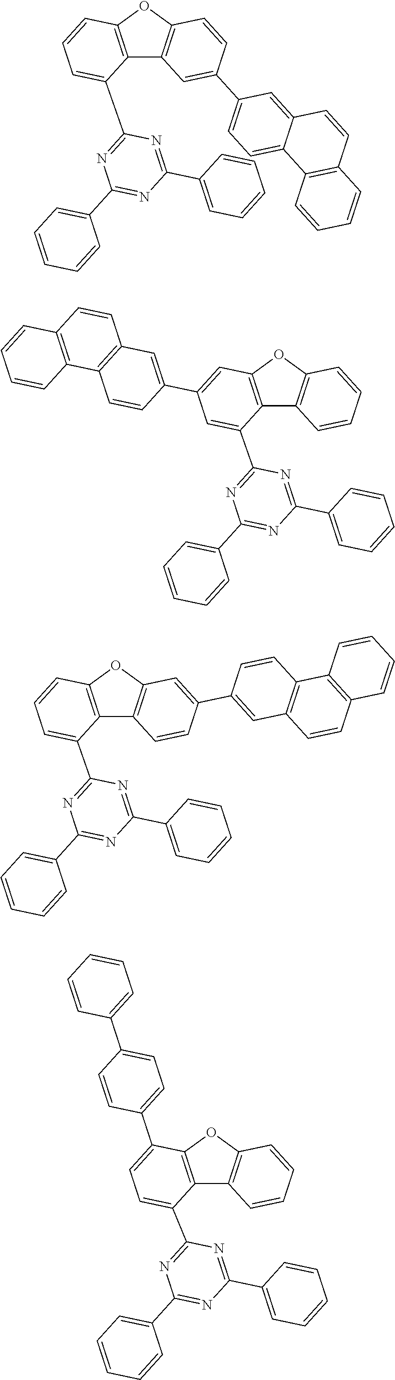

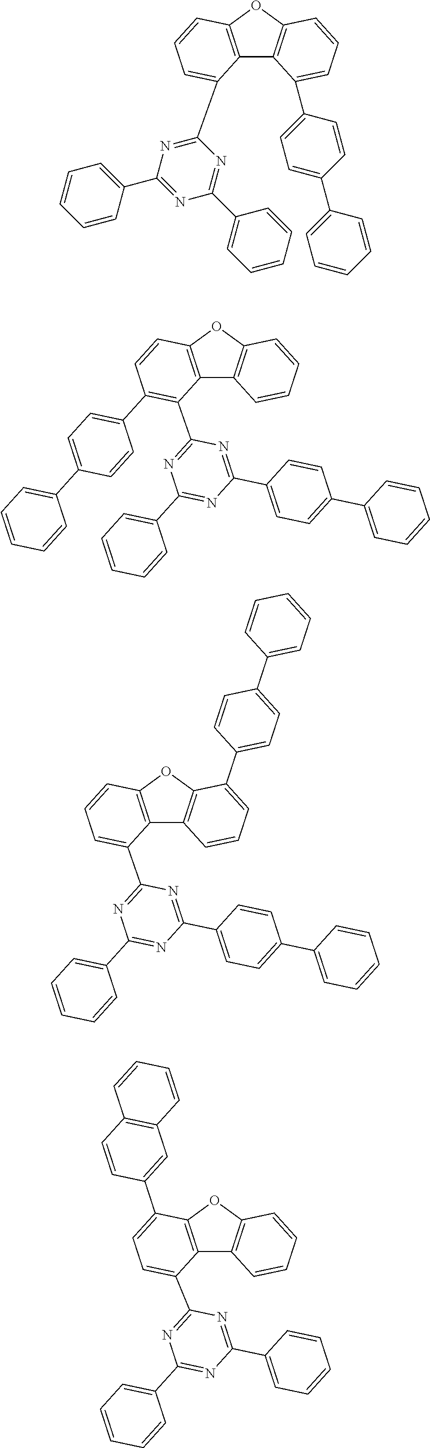 [SMILES: c1ccc(-c2ccc(-c3cccc4oc5cccc(-c6nc(-c7ccccc7)nc(-c7ccccc7)n6)c5c34)cc2)cc1 ; c1ccc(-c2ccc(-c3nc(-c4ccccc4)nc(-c4c(-c5ccc(-c6ccccc6)cc5)ccc5oc6ccccc6c45)n3)cc2)cc1 ; c1ccc(-c2ccc(-c3nc(-c4ccccc4)nc(-c4cccc5oc6c(-c7ccc(-c8ccccc8)cc7)cccc6c45)n3)cc2)cc1 ; c1ccc(-c2nc(-c3ccccc3)nc(-c3ccc(-c4ccc5ccccc5c4)c4oc5ccccc5c34)n2)cc1]

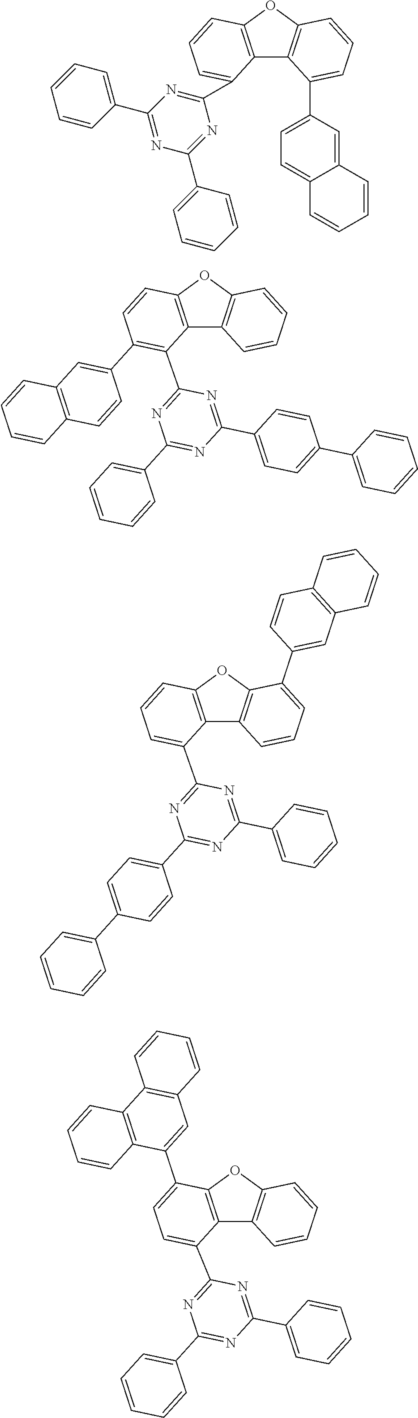

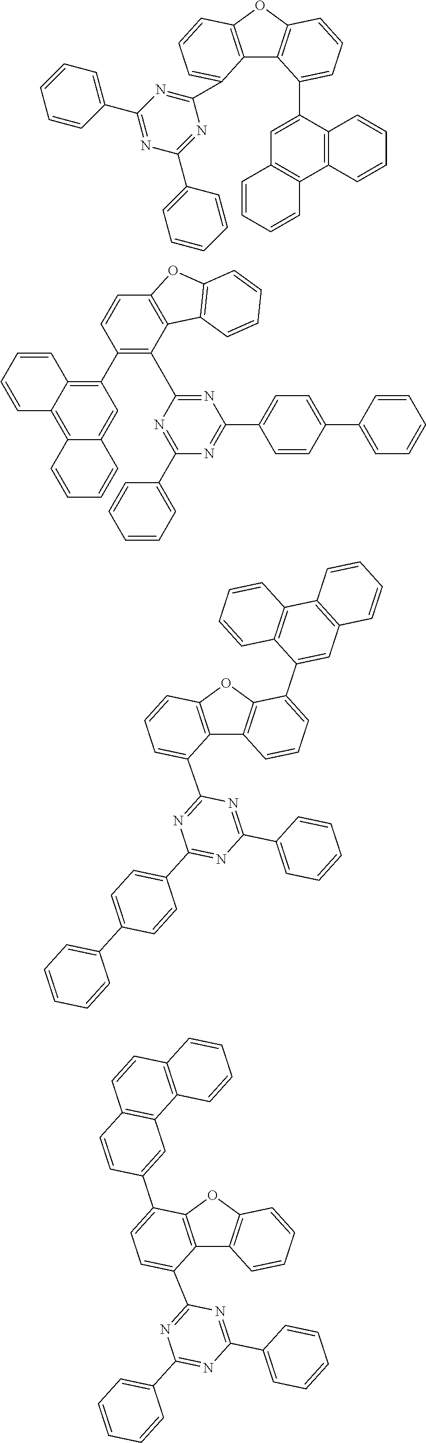

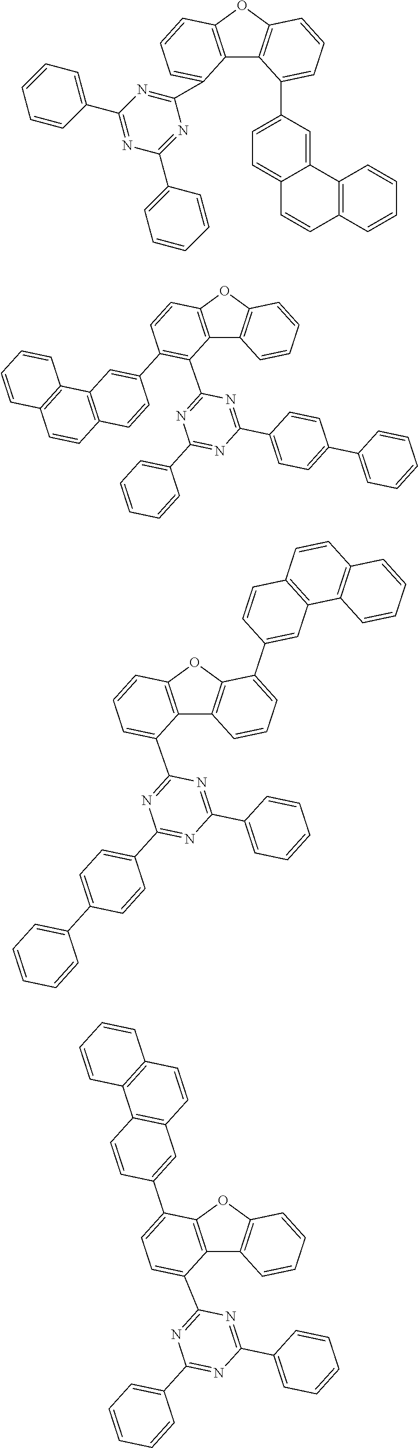 [SMILES: c1ccc(-c2nc(-c3ccccc3)nc(-c3cccc4oc5cccc(-c6ccc7ccc8ccccc8c7c6)c5c34)n2)cc1 ; c1ccc(-c2ccc(-c3nc(-c4ccccc4)nc(-c4c(-c5ccc6ccc7ccccc7c6c5)ccc5oc6ccccc6c45)n3)cc2)cc1 ; c1ccc(-c2ccc(-c3nc(-c4ccccc4)nc(-c4cccc5oc6c(-c7ccc8ccc9ccccc9c8c7)cccc6c45)n3)cc2)cc1 ; c1ccc(-c2nc(-c3ccccc3)nc(-c3ccc(-c4ccc5c(ccc6ccccc65)c4)c4oc5ccccc5c34)n2)cc1]

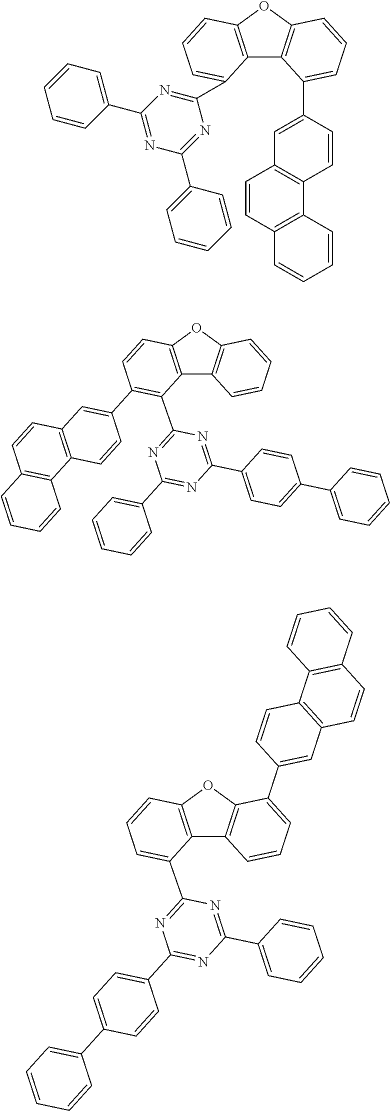 [SMILES: c1ccc(-c2nc(-c3ccccc3)nc(-c3cccc4oc5cccc(-c6ccc7c(ccc8ccccc87)c6)c5c34)n2)cc1 ; c1ccc(-c2ccc(-c3nc(-c4ccccc4)nc(-c4c(-c5ccc6c(ccc7ccccc76)c5)ccc5oc6ccccc6c45)n3)cc2)cc1 ; c1ccc(-c2ccc(-c3nc(-c4ccccc4)nc(-c4cccc5oc6c(-c7ccc8c(ccc9ccccc98)c7)cccc6c45)n3)cc2)cc1]

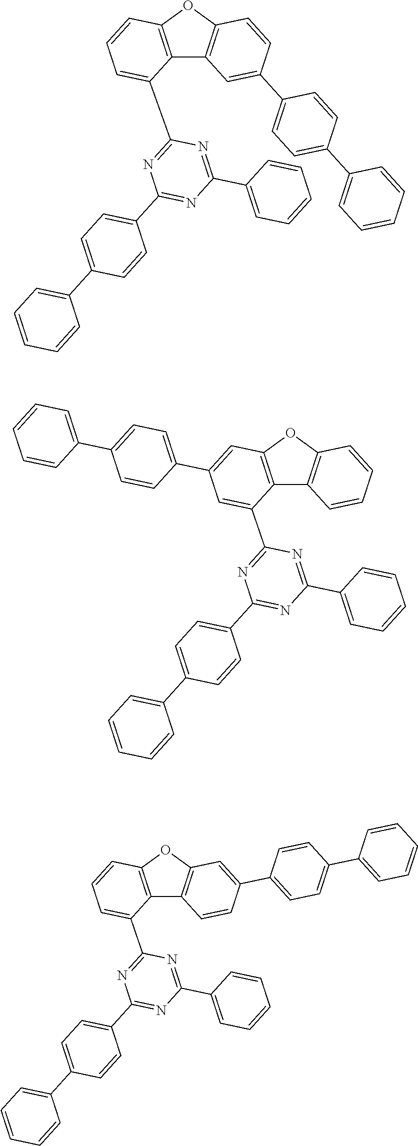 [SMILES: c1ccc(-c2ccc(-c3ccc4oc5cccc(-c6nc(-c7ccccc7)nc(-c7ccc(-c8ccccc8)cc7)n6)c5c4c3)cc2)cc1 ; c1ccc(-c2ccc(-c3cc(-c4nc(-c5ccccc5)nc(-c5ccc(-c6ccccc6)cc5)n4)c4c(c3)oc3ccccc34)cc2)cc1 ; c1ccc(-c2ccc(-c3ccc4c(c3)oc3cccc(-c5nc(-c6ccccc6)nc(-c6ccc(-c7ccccc7)cc6)n5)c34)cc2)cc1]

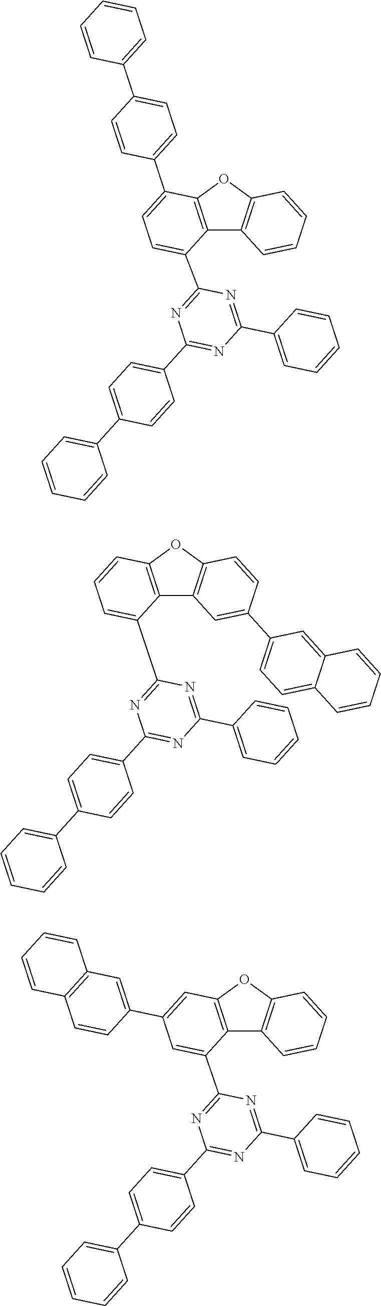

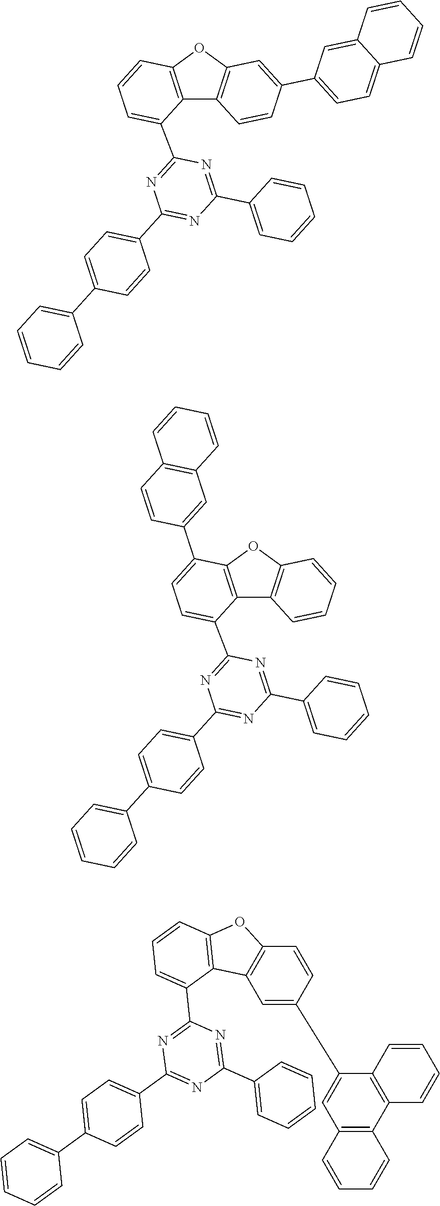 [SMILES: c1ccc(-c2ccc(-c3nc(-c4ccccc4)nc(-c4cccc5oc6cc(-c7ccc8ccccc8c7)ccc6c45)n3)cc2)cc1 ; c1ccc(-c2ccc(-c3nc(-c4ccccc4)nc(-c4ccc(-c5ccc6ccccc6c5)c5oc6ccccc6c45)n3)cc2)cc1 ; c1ccc(-c2ccc(-c3nc(-c4ccccc4)nc(-c4cccc5oc6ccc(-c7cc8ccccc8c8ccccc78)cc6c45)n3)cc2)cc1]

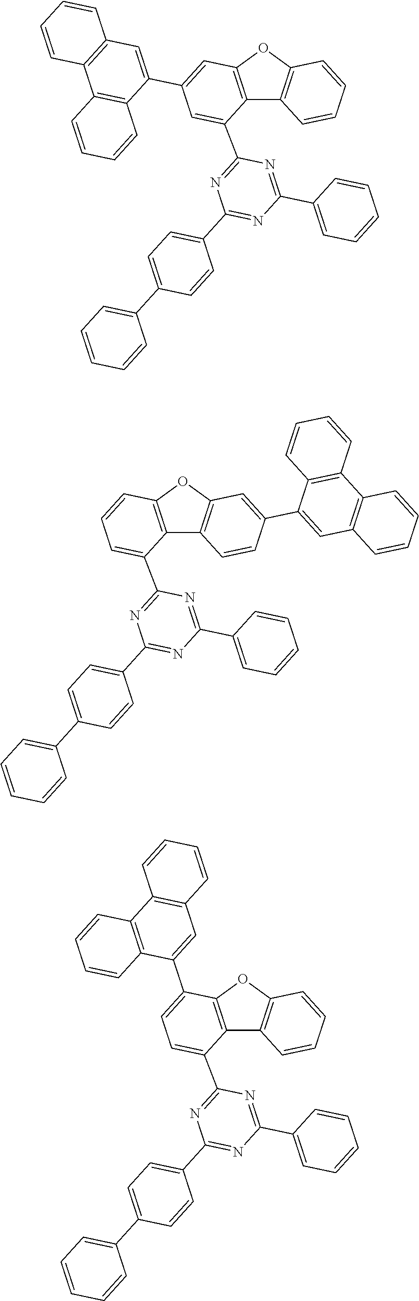 [SMILES: c1ccc(-c2ccc(-c3nc(-c4ccccc4)nc(-c4cc(-c5cc6ccccc6c6ccccc56)cc5oc6ccccc6c45)n3)cc2)cc1 ; c1ccc(-c2ccc(-c3nc(-c4ccccc4)nc(-c4cccc5oc6cc(-c7cc8ccccc8c8ccccc78)ccc6c45)n3)cc2)cc1 ; c1ccc(-c2ccc(-c3nc(-c4ccccc4)nc(-c4ccc(-c5cc6ccccc6c6ccccc56)c5oc6ccccc6c45)n3)cc2)cc1]

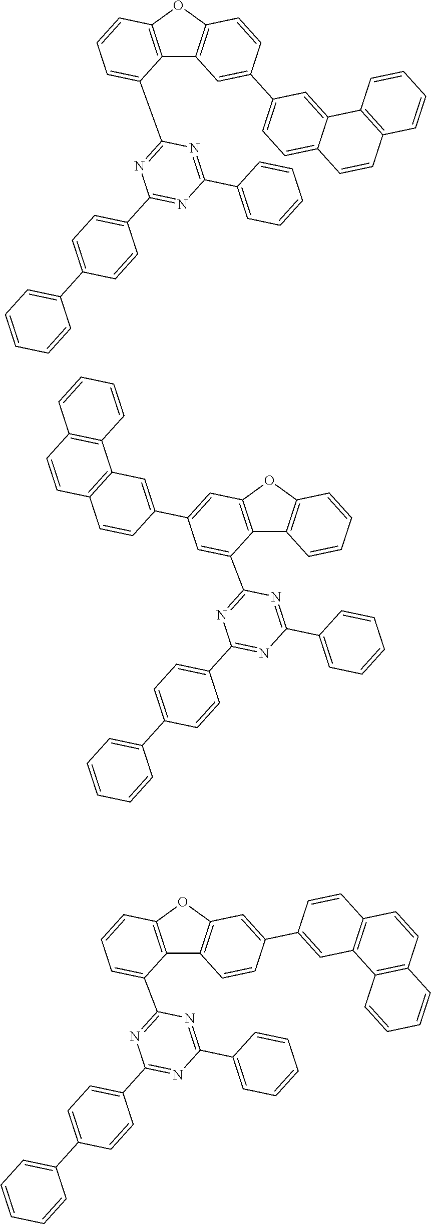

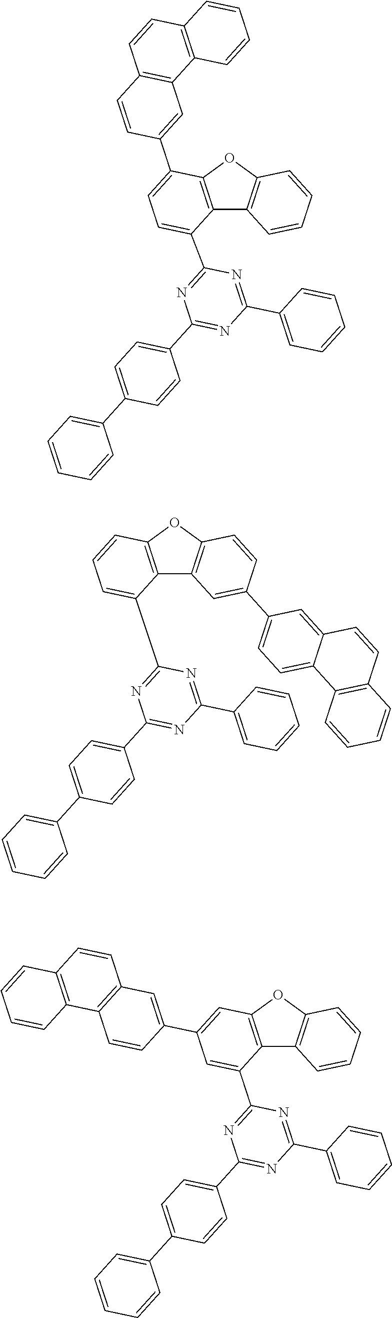 [SMILES: c1ccc(-c2ccc(-c3nc(-c4ccccc4)nc(-c4ccc(-c5ccc6ccc7ccccc7c6c5)c5oc6ccccc6c45)n3)cc2)cc1 ; c1ccc(-c2ccc(-c3nc(-c4ccccc4)nc(-c4cccc5oc6ccc(-c7ccc8c(ccc9ccccc98)c7)cc6c45)n3)cc2)cc1 ; c1ccc(-c2ccc(-c3nc(-c4ccccc4)nc(-c4cc(-c5ccc6c(ccc7ccccc76)c5)cc5oc6ccccc6c45)n3)cc2)cc1]

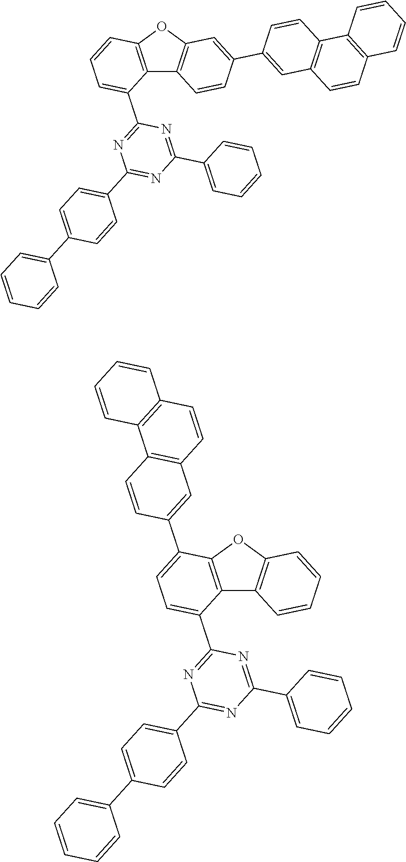 [SMILES: c1ccc(-c2ccc(-c3nc(-c4ccccc4)nc(-c4cccc5oc6cc(-c7ccc8c(ccc9ccccc98)c7)ccc6c45)n3)cc2)cc1 ; c1ccc(-c2ccc(-c3nc(-c4ccccc4)nc(-c4ccc(-c5ccc6c(ccc7ccccc76)c5)c5oc6ccccc6c45)n3)cc2)cc1]

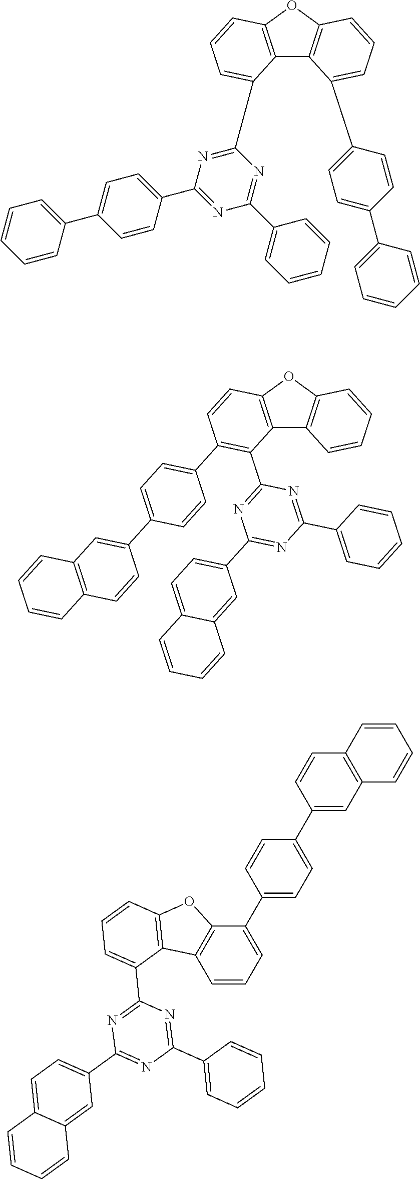 [SMILES: c1ccc(-c2ccc(-c3nc(-c4ccccc4)nc(-c4cccc5oc6cccc(-c7ccc(-c8ccccc8)cc7)c6c45)n3)cc2)cc1 ; c1ccc(-c2nc(-c3ccc4ccccc4c3)nc(-c3c(-c4ccc(-c5ccc6ccccc6c5)cc4)ccc4oc5ccccc5c34)n2)cc1 ; c1ccc(-c2nc(-c3ccc4ccccc4c3)nc(-c3cccc4oc5c(-c6ccc(-c7ccc8ccccc8c7)cc6)cccc5c34)n2)cc1]

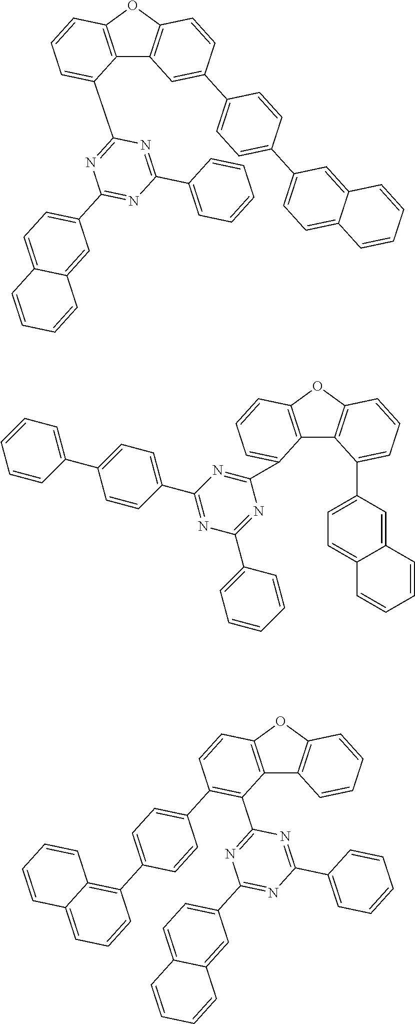 [SMILES: c1ccc(-c2nc(-c3ccc4ccccc4c3)nc(-c3cccc4oc5ccc(-c6ccc(-c7ccc8ccccc8c7)cc6)cc5c34)n2)cc1 ; c1ccc(-c2ccc(-c3nc(-c4ccccc4)nc(-c4cccc5oc6cccc(-c7ccc8ccccc8c7)c6c45)n3)cc2)cc1 ; c1ccc(-c2nc(-c3ccc4ccccc4c3)nc(-c3c(-c4ccc(-c5cccc6ccccc56)cc4)ccc4oc5ccccc5c34)n2)cc1]

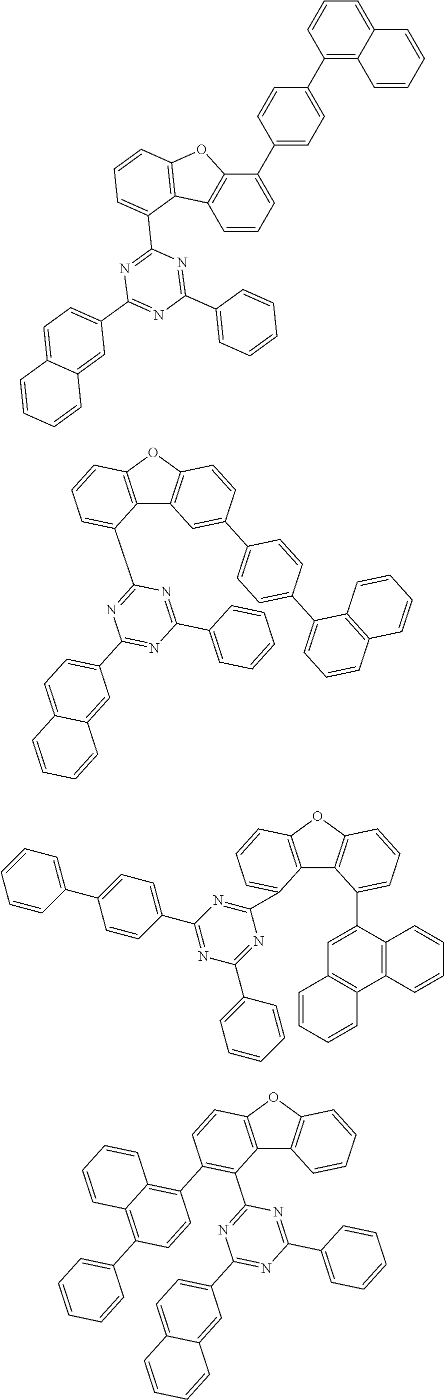

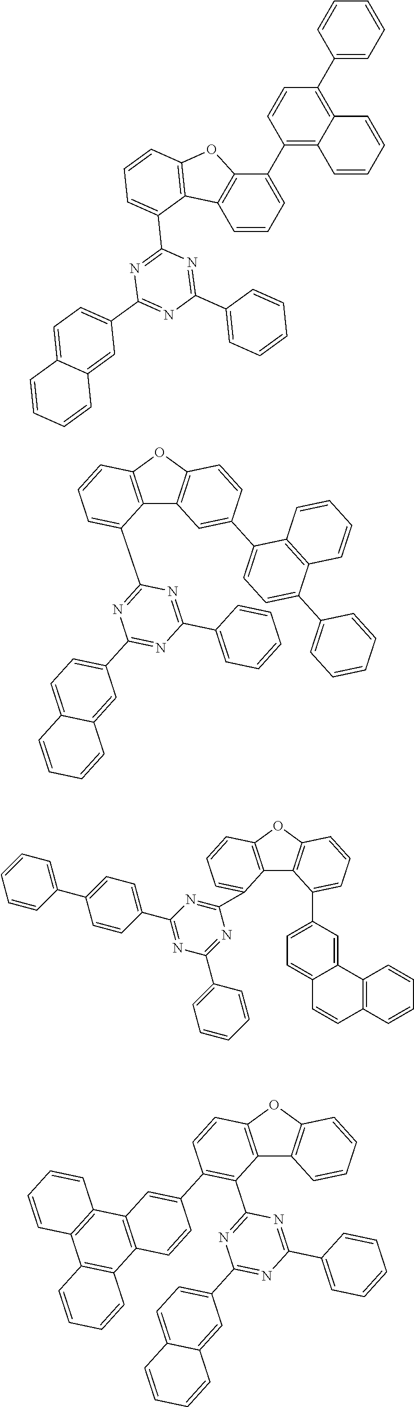 [SMILES: c1ccc(-c2nc(-c3ccc4ccccc4c3)nc(-c3cccc4oc5c(-c6ccc(-c7ccccc7)c7ccccc67)cccc5c34)n2)cc1 ; c1ccc(-c2nc(-c3ccc4ccccc4c3)nc(-c3cccc4oc5ccc(-c6ccc(-c7ccccc7)c7ccccc67)cc5c34)n2)cc1 ; c1ccc(-c2ccc(-c3nc(-c4ccccc4)nc(-c4cccc5oc6cccc(-c7ccc8ccc9ccccc9c8c7)c6c45)n3)cc2)cc1 ; c1ccc(-c2nc(-c3ccc4ccccc4c3)nc(-c3c(-c4ccc5c6ccccc6c6ccccc6c5c4)ccc4oc5ccccc5c34)n2)cc1]

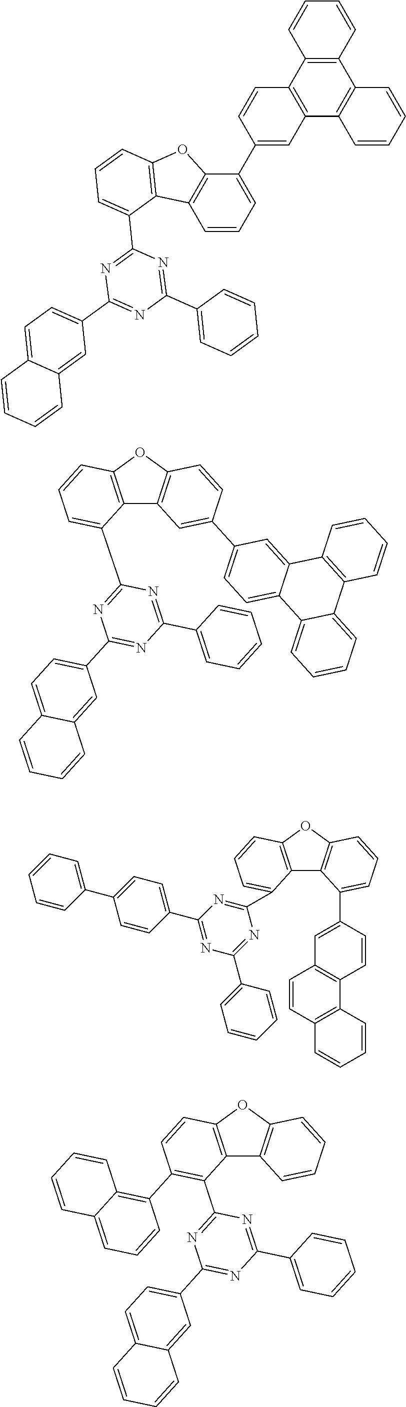 [SMILES: c1ccc(-c2nc(-c3ccc4ccccc4c3)nc(-c3cccc4oc5c(-c6ccc7c8ccccc8c8ccccc8c7c6)cccc5c34)n2)cc1 ; c1ccc(-c2nc(-c3ccc4ccccc4c3)nc(-c3cccc4oc5ccc(-c6ccc7c8ccccc8c8ccccc8c7c6)cc5c34)n2)cc1 ; c1ccc(-c2ccc(-c3nc(-c4ccccc4)nc(-c4cccc5oc6cccc(-c7ccc8c(ccc9ccccc98)c7)c6c45)n3)cc2)cc1 ; c1ccc(-c2nc(-c3ccc4ccccc4c3)nc(-c3c(-c4cccc5ccccc45)ccc4oc5ccccc5c34)n2)cc1]

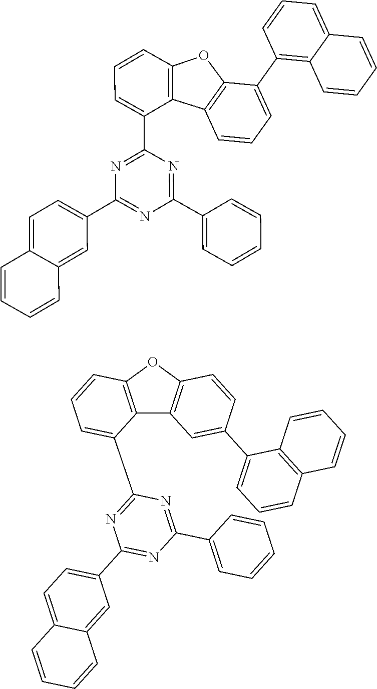 [SMILES: c1ccc(-c2nc(-c3ccc4ccccc4c3)nc(-c3cccc4oc5c(-c6cccc7ccccc67)cccc5c34)n2)cc1 ; c1ccc(-c2nc(-c3ccc4ccccc4c3)nc(-c3cccc4oc5ccc(-c6cccc7ccccc67)cc5c34)n2)cc1]

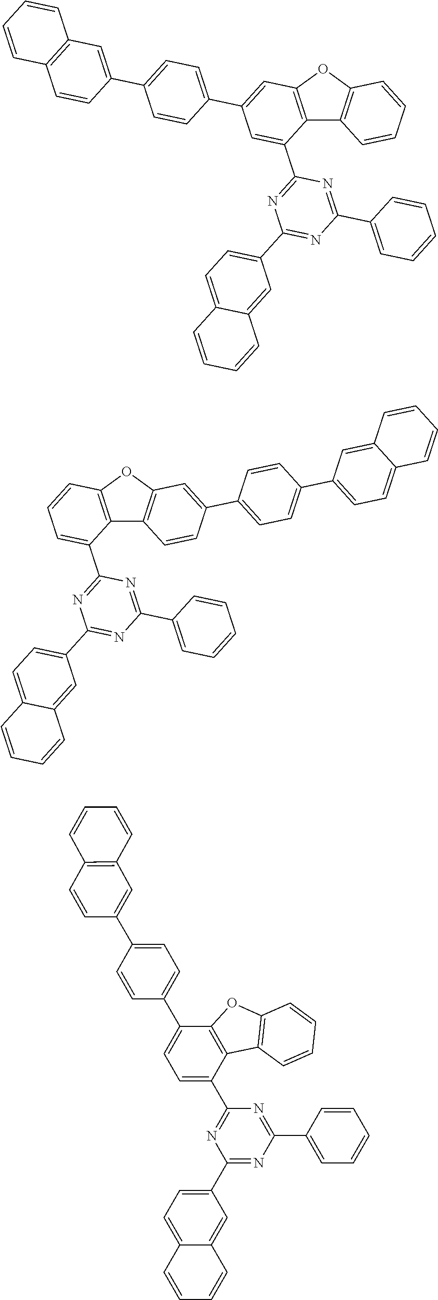 [SMILES: c1ccc(-c2nc(-c3ccc4ccccc4c3)nc(-c3cc(-c4ccc(-c5ccc6ccccc6c5)cc4)cc4oc5ccccc5c34)n2)cc1 ; c1ccc(-c2nc(-c3ccc4ccccc4c3)nc(-c3cccc4oc5cc(-c6ccc(-c7ccc8ccccc8c7)cc6)ccc5c34)n2)cc1 ; c1ccc(-c2nc(-c3ccc4ccccc4c3)nc(-c3ccc(-c4ccc(-c5ccc6ccccc6c5)cc4)c4oc5ccccc5c34)n2)cc1]

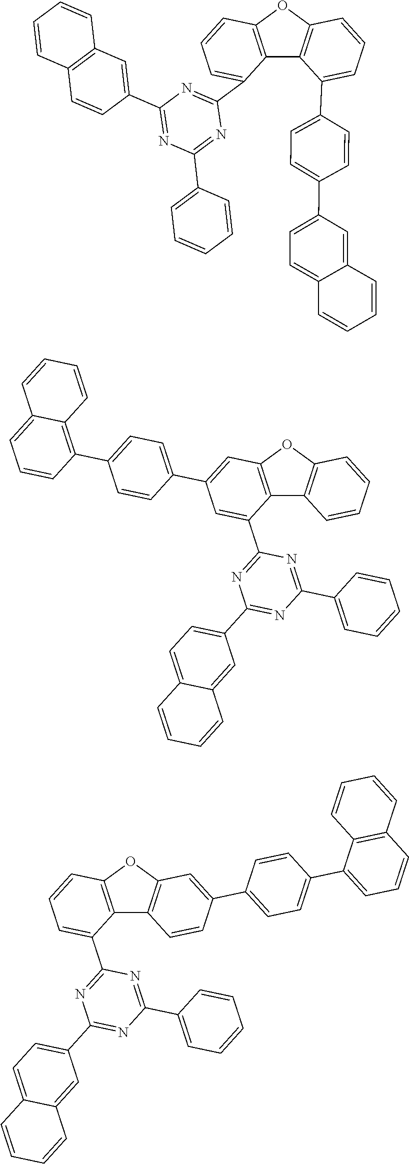 [SMILES: c1ccc(-c2nc(-c3ccc4ccccc4c3)nc(-c3cccc4oc5cccc(-c6ccc(-c7ccc8ccccc8c7)cc6)c5c34)n2)cc1 ; c1ccc(-c2nc(-c3ccc4ccccc4c3)nc(-c3cc(-c4ccc(-c5cccc6ccccc56)cc4)cc4oc5ccccc5c34)n2)cc1 ; c1ccc(-c2nc(-c3ccc4ccccc4c3)nc(-c3cccc4oc5cc(-c6ccc(-c7cccc8ccccc78)cc6)ccc5c34)n2)cc1]

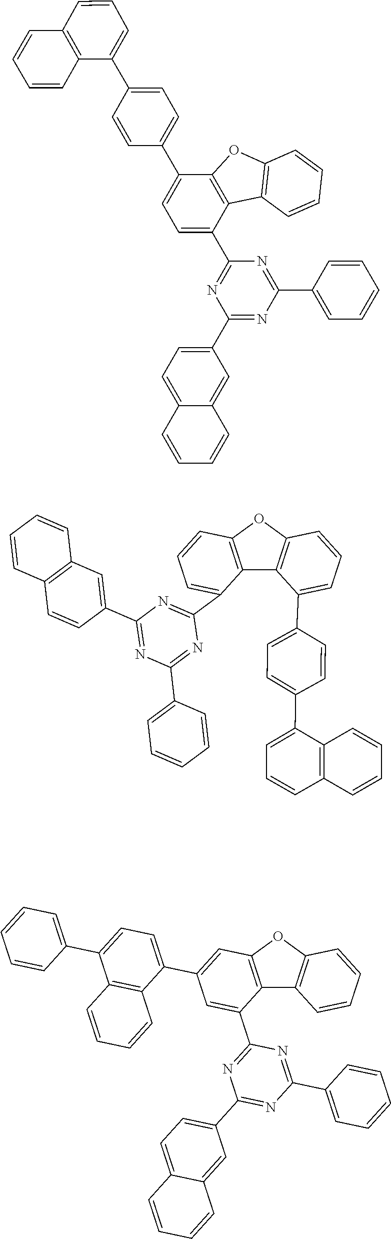 [SMILES: c1ccc(-c2nc(-c3ccc4ccccc4c3)nc(-c3ccc(-c4ccc(-c5cccc6ccccc56)cc4)c4oc5ccccc5c34)n2)cc1 ; c1ccc(-c2nc(-c3ccc4ccccc4c3)nc(-c3cccc4oc5cccc(-c6ccc(-c7cccc8ccccc78)cc6)c5c34)n2)cc1 ; c1ccc(-c2nc(-c3ccc4ccccc4c3)nc(-c3cc(-c4ccc(-c5ccccc5)c5ccccc45)cc4oc5ccccc5c34)n2)cc1]

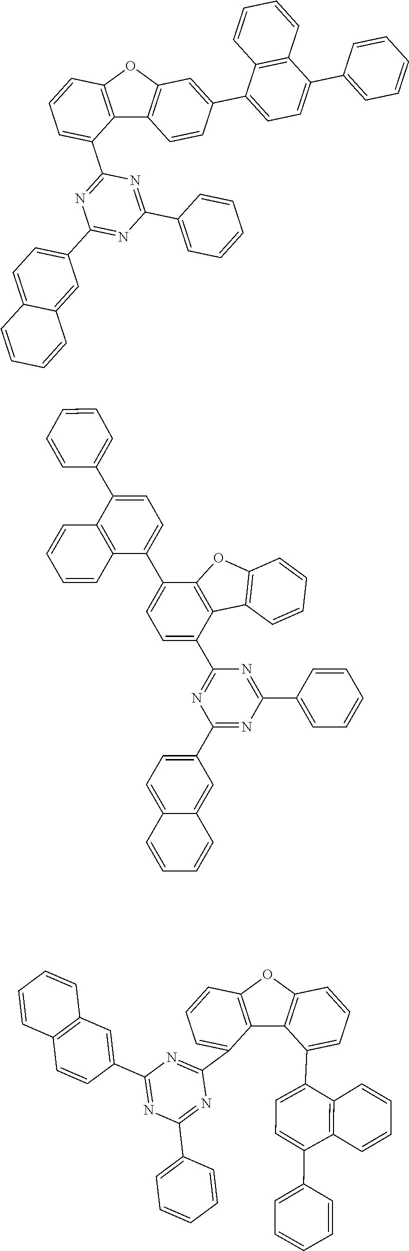

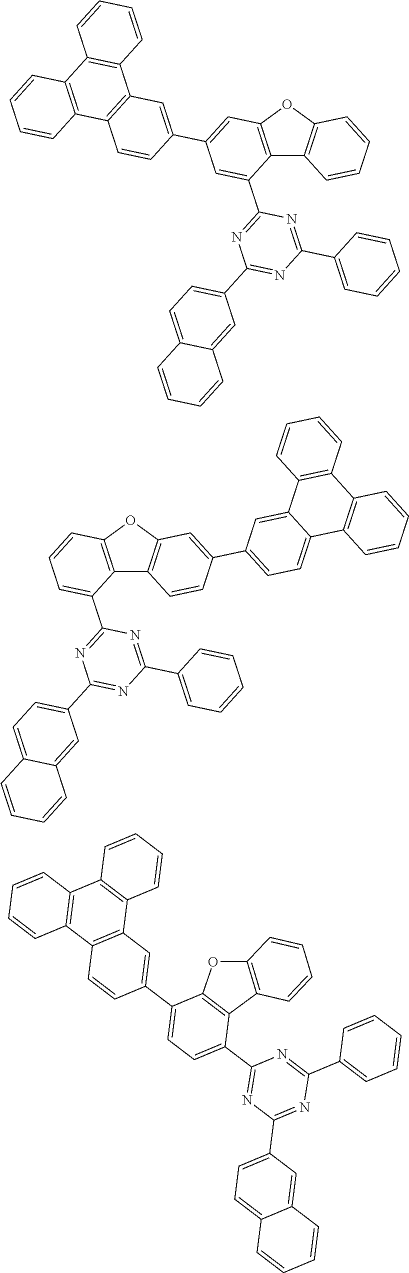 [SMILES: c1ccc(-c2nc(-c3ccc4ccccc4c3)nc(-c3cc(-c4ccc5c6ccccc6c6ccccc6c5c4)cc4oc5ccccc5c34)n2)cc1 ; c1ccc(-c2nc(-c3ccc4ccccc4c3)nc(-c3cccc4oc5cc(-c6ccc7c8ccccc8c8ccccc8c7c6)ccc5c34)n2)cc1 ; c1ccc(-c2nc(-c3ccc4ccccc4c3)nc(-c3ccc(-c4ccc5c6ccccc6c6ccccc6c5c4)c4oc5ccccc5c34)n2)cc1]

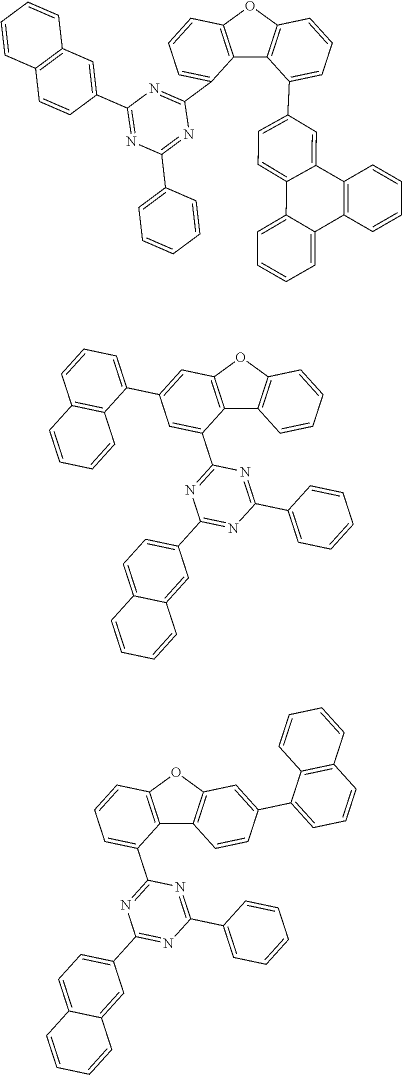 [SMILES: c1ccc(-c2nc(-c3ccc4ccccc4c3)nc(-c3cccc4oc5cccc(-c6ccc7c8ccccc8c8ccccc8c7c6)c5c34)n2)cc1 ; c1ccc(-c2nc(-c3ccc4ccccc4c3)nc(-c3cc(-c4cccc5ccccc45)cc4oc5ccccc5c34)n2)cc1 ; c1ccc(-c2nc(-c3ccc4ccccc4c3)nc(-c3cccc4oc5cc(-c6cccc7ccccc67)ccc5c34)n2)cc1]

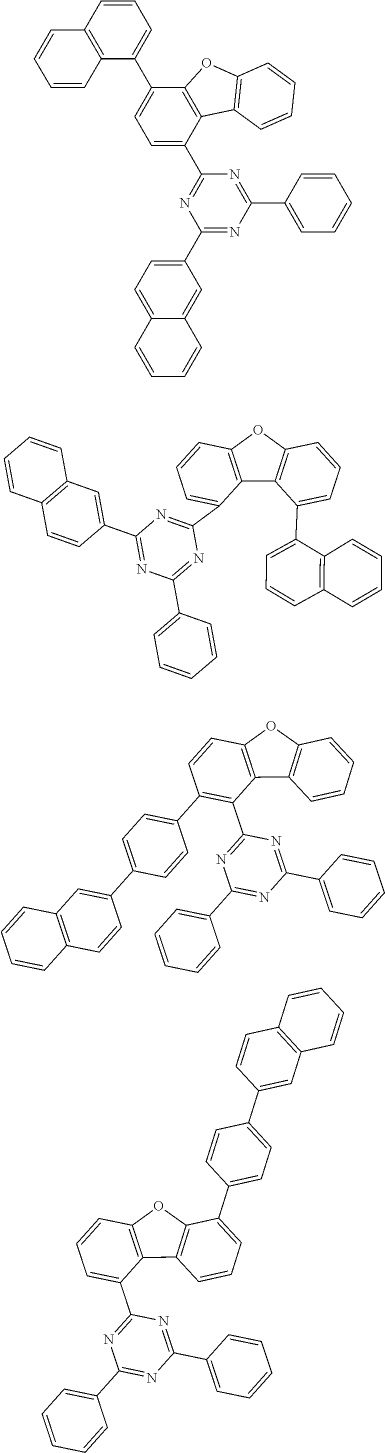 [SMILES: c1ccc(-c2nc(-c3ccc4ccccc4c3)nc(-c3ccc(-c4cccc5ccccc45)c4oc5ccccc5c34)n2)cc1 ; c1ccc(-c2nc(-c3ccc4ccccc4c3)nc(-c3cccc4oc5cccc(-c6cccc7ccccc67)c5c34)n2)cc1 ; c1ccc(-c2nc(-c3ccccc3)nc(-c3c(-c4ccc(-c5ccc6ccccc6c5)cc4)ccc4oc5ccccc5c34)n2)cc1 ; c1ccc(-c2nc(-c3ccccc3)nc(-c3cccc4oc5c(-c6ccc(-c7ccc8ccccc8c7)cc6)cccc5c34)n2)cc1]

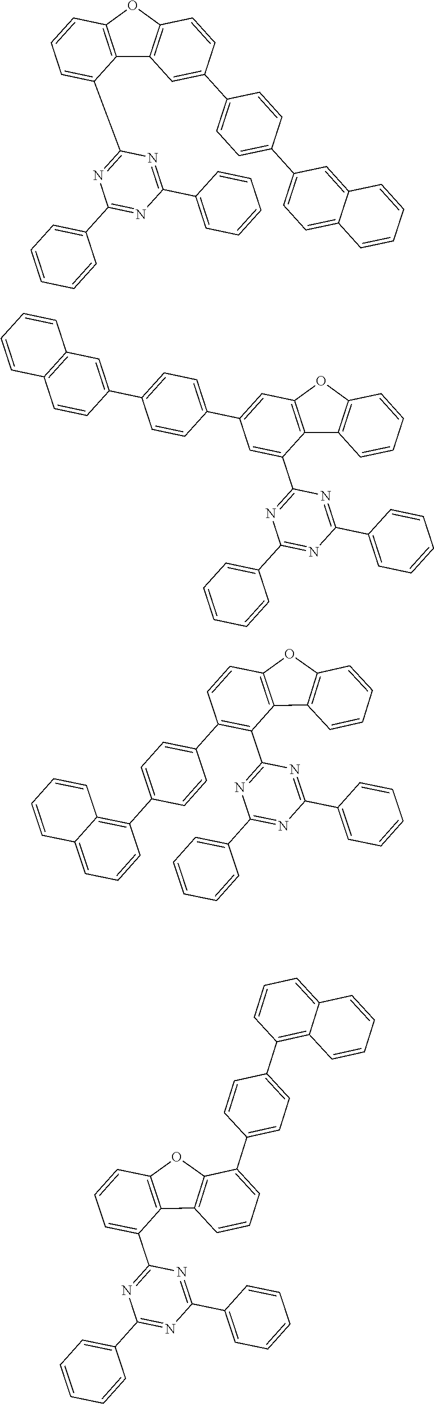

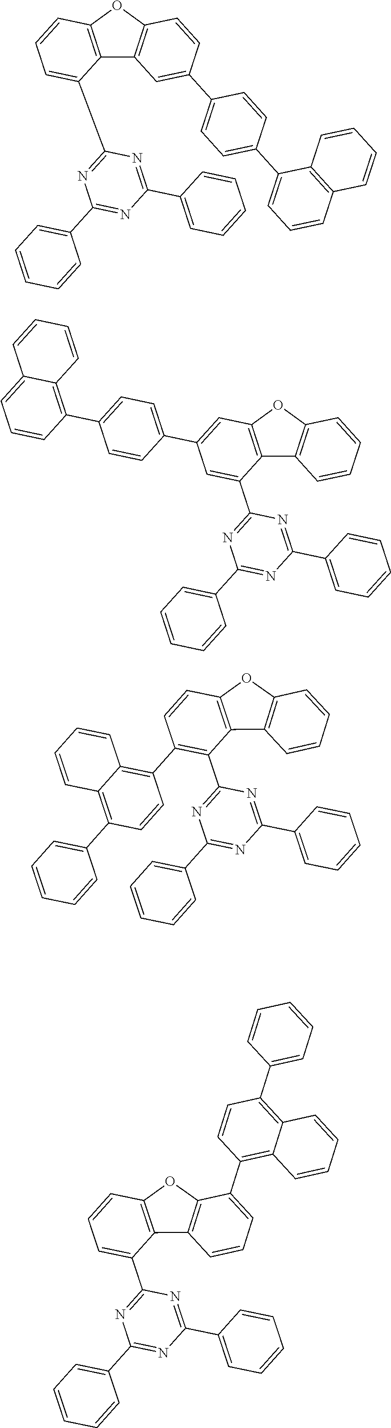

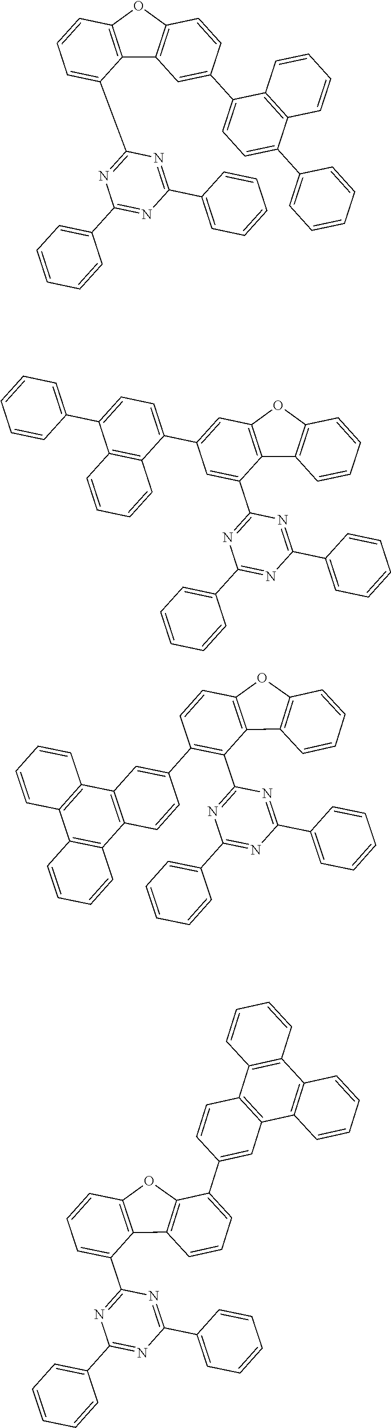

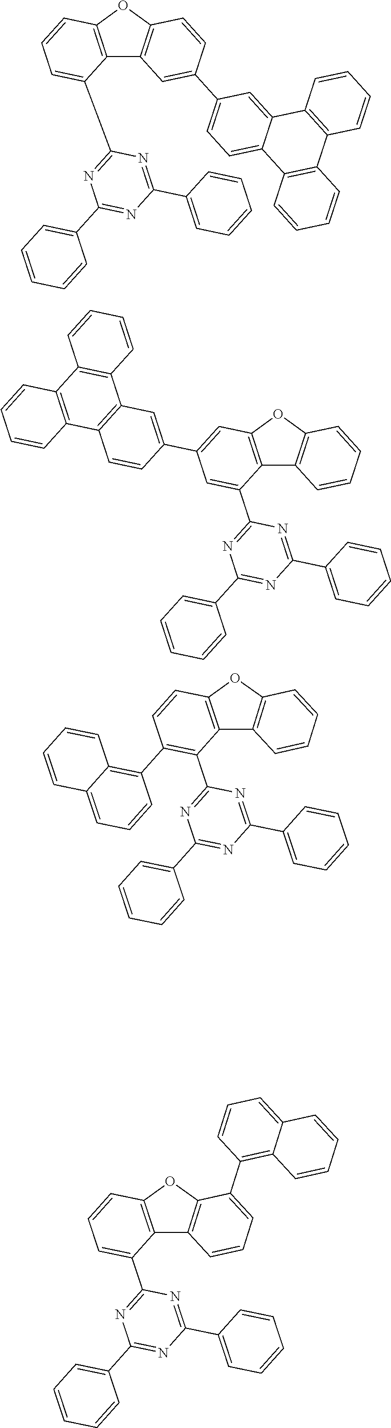

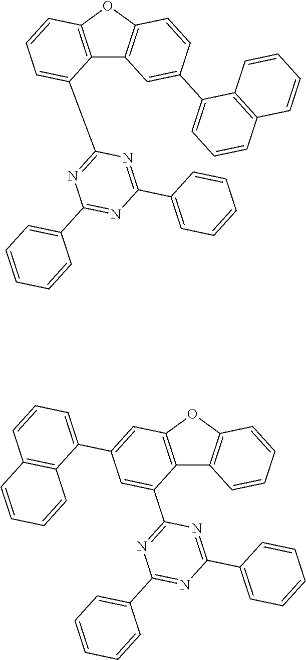 [SMILES: c1ccc(-c2nc(-c3ccccc3)nc(-c3cccc4oc5ccc(-c6cccc7ccccc67)cc5c34)n2)cc1 ; c1ccc(-c2nc(-c3ccccc3)nc(-c3cc(-c4cccc5ccccc45)cc4oc5ccccc5c34)n2)cc1]

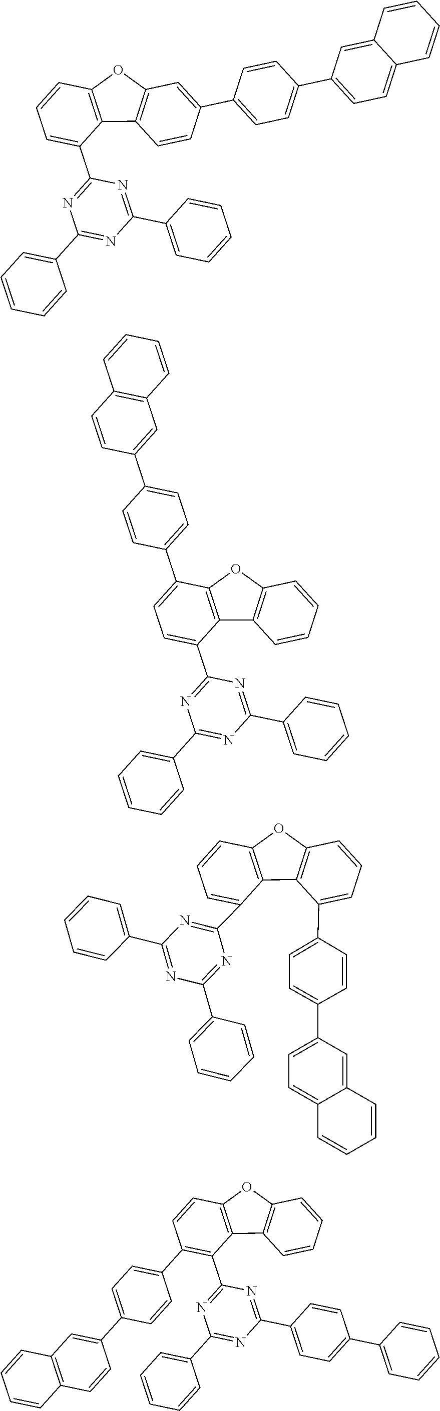 [SMILES: c1ccc(-c2nc(-c3ccccc3)nc(-c3cccc4oc5cc(-c6ccc(-c7ccc8ccccc8c7)cc6)ccc5c34)n2)cc1 ; c1ccc(-c2nc(-c3ccccc3)nc(-c3ccc(-c4ccc(-c5ccc6ccccc6c5)cc4)c4oc5ccccc5c34)n2)cc1 ; c1ccc(-c2nc(-c3ccccc3)nc(-c3cccc4oc5cccc(-c6ccc(-c7ccc8ccccc8c7)cc6)c5c34)n2)cc1 ; c1ccc(-c2ccc(-c3nc(-c4ccccc4)nc(-c4c(-c5ccc(-c6ccc7ccccc7c6)cc5)ccc5oc6ccccc6c45)n3)cc2)cc1]

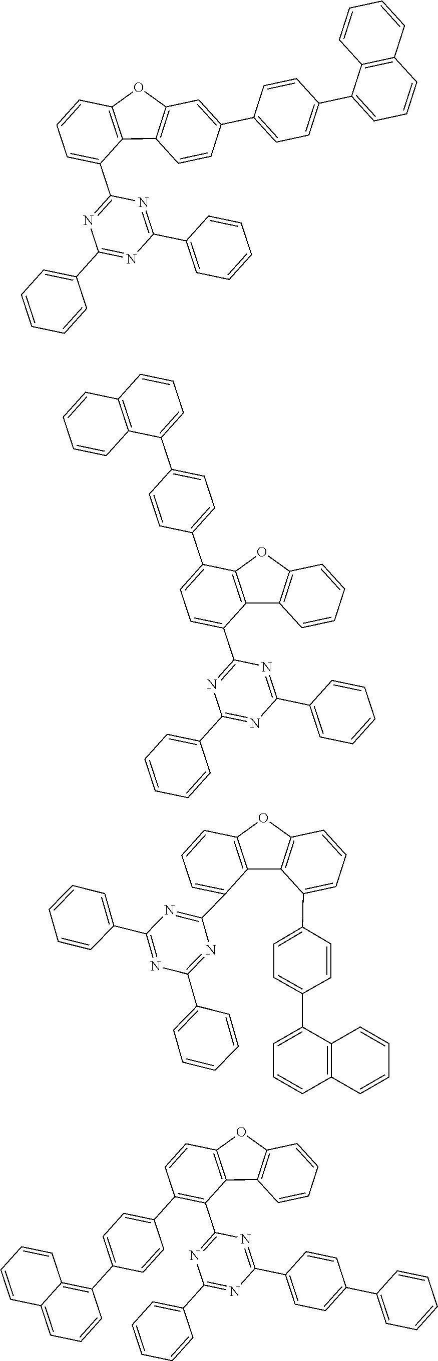

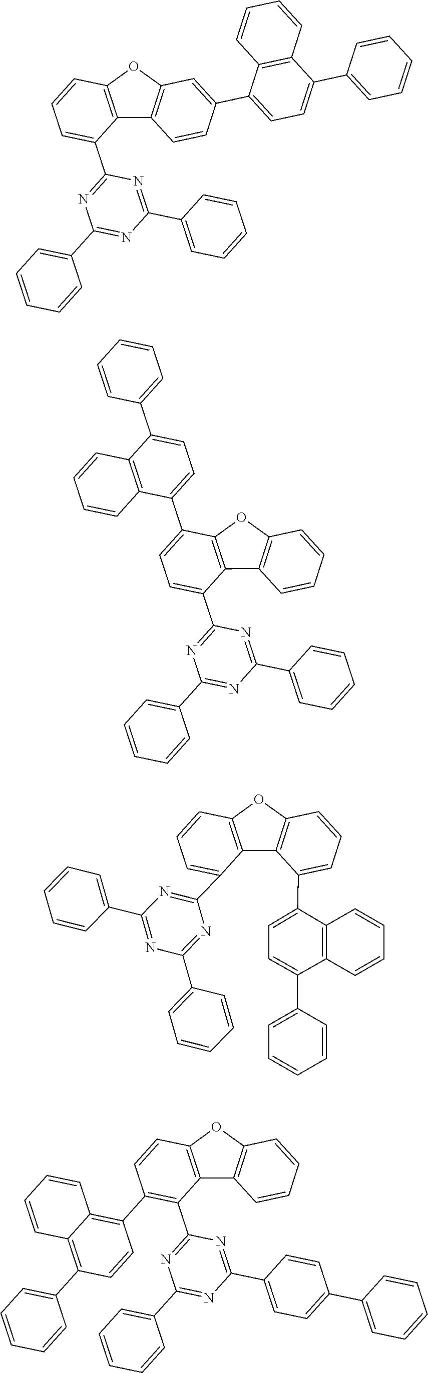 [SMILES: c1ccc(-c2nc(-c3ccccc3)nc(-c3cccc4oc5cc(-c6ccc(-c7ccccc7)c7ccccc67)ccc5c34)n2)cc1 ; c1ccc(-c2nc(-c3ccccc3)nc(-c3ccc(-c4ccc(-c5ccccc5)c5ccccc45)c4oc5ccccc5c34)n2)cc1 ; c1ccc(-c2nc(-c3ccccc3)nc(-c3cccc4oc5cccc(-c6ccc(-c7ccccc7)c7ccccc67)c5c34)n2)cc1 ; c1ccc(-c2ccc(-c3nc(-c4ccccc4)nc(-c4c(-c5ccc(-c6ccccc6)c6ccccc56)ccc5oc6ccccc6c45)n3)cc2)cc1]

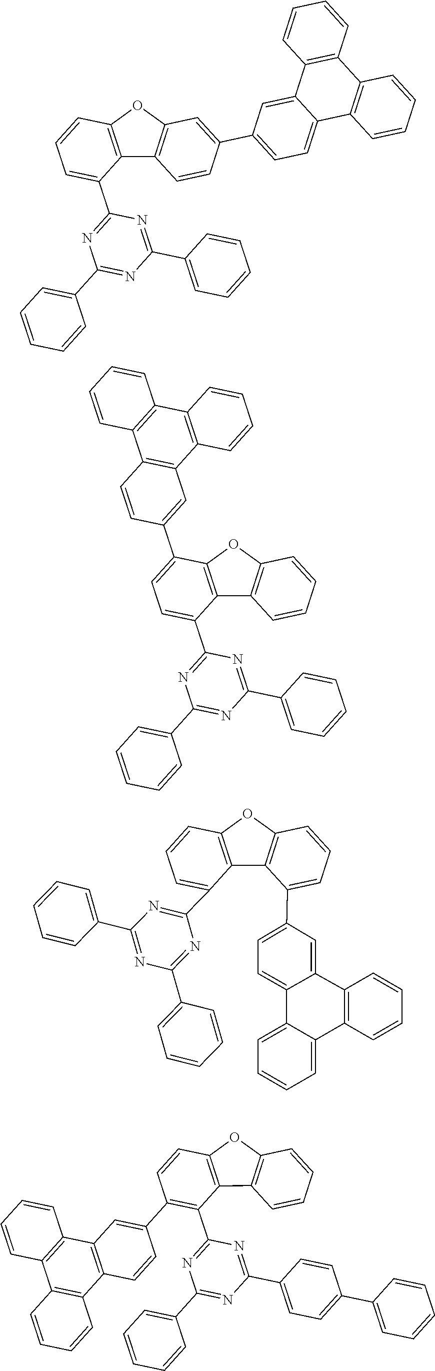

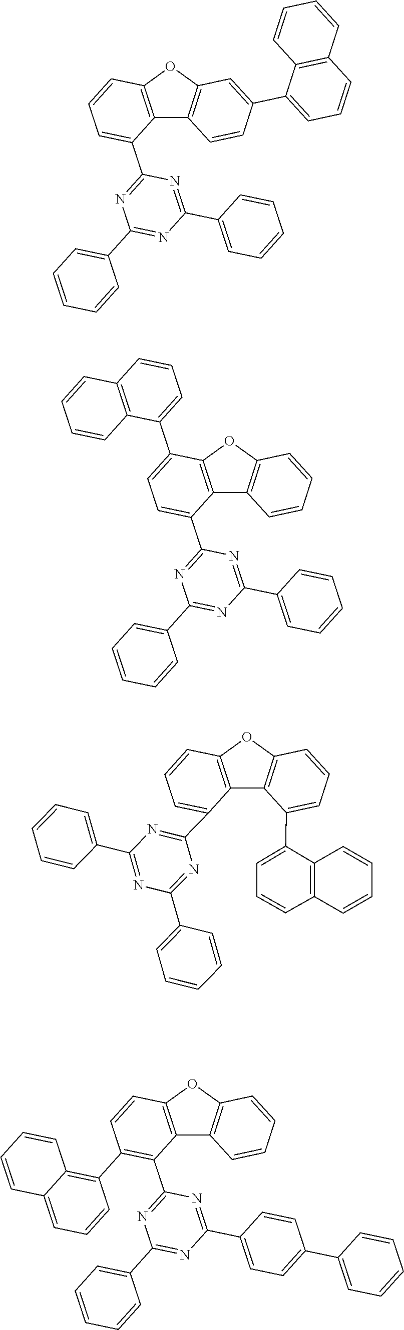 [SMILES: c1ccc(-c2nc(-c3ccccc3)nc(-c3cccc4oc5cc(-c6cccc7ccccc67)ccc5c34)n2)cc1 ; c1ccc(-c2nc(-c3ccccc3)nc(-c3ccc(-c4cccc5ccccc45)c4oc5ccccc5c34)n2)cc1 ; c1ccc(-c2nc(-c3ccccc3)nc(-c3cccc4oc5cccc(-c6cccc7ccccc67)c5c34)n2)cc1 ; c1ccc(-c2ccc(-c3nc(-c4ccccc4)nc(-c4c(-c5cccc6ccccc56)ccc5oc6ccccc6c45)n3)cc2)cc1]

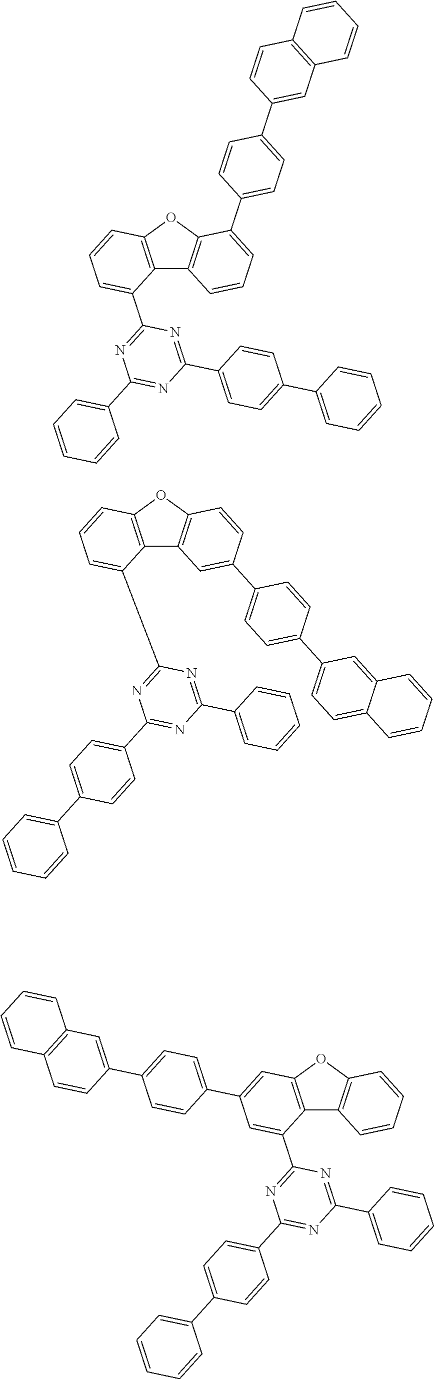

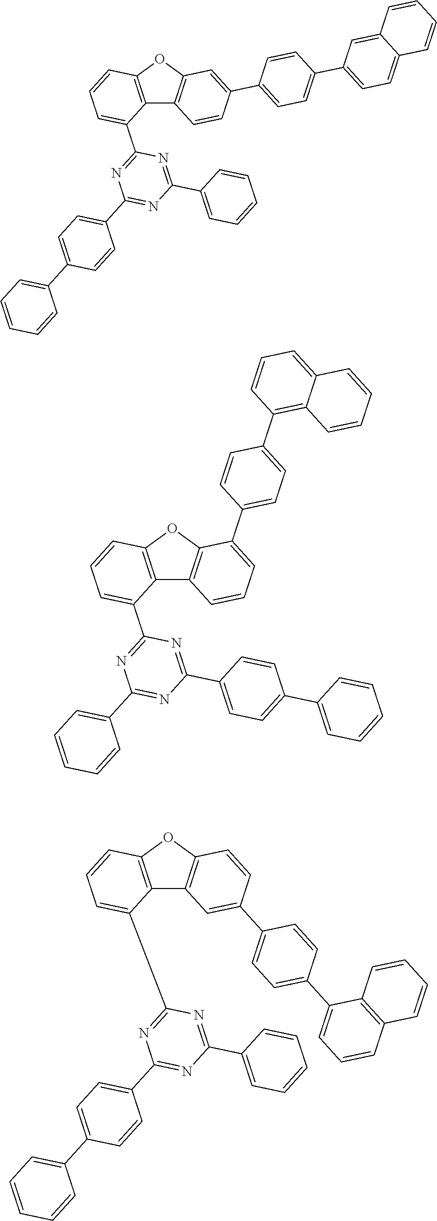 [SMILES: c1ccc(-c2ccc(-c3nc(-c4ccccc4)nc(-c4cccc5oc6cc(-c7ccc(-c8ccc9ccccc9c8)cc7)ccc6c45)n3)cc2)cc1 ; c1ccc(-c2ccc(-c3nc(-c4ccccc4)nc(-c4cccc5oc6c(-c7ccc(-c8cccc9ccccc89)cc7)cccc6c45)n3)cc2)cc1 ; c1ccc(-c2ccc(-c3nc(-c4ccccc4)nc(-c4cccc5oc6ccc(-c7ccc(-c8cccc9ccccc89)cc7)cc6c45)n3)cc2)cc1]

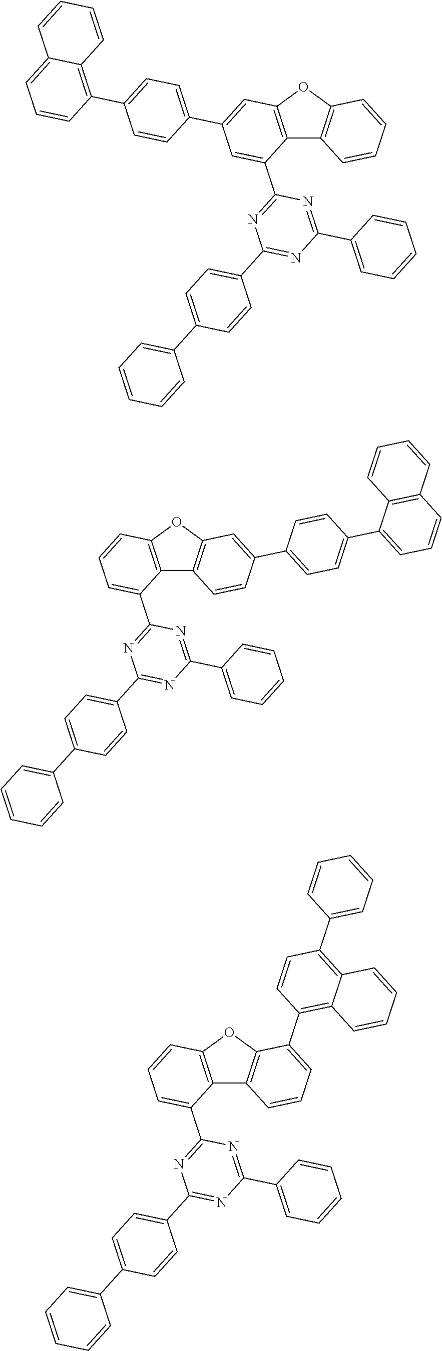 [SMILES: c1ccc(-c2ccc(-c3nc(-c4ccccc4)nc(-c4cc(-c5ccc(-c6cccc7ccccc67)cc5)cc5oc6ccccc6c45)n3)cc2)cc1 ; c1ccc(-c2ccc(-c3nc(-c4ccccc4)nc(-c4cccc5oc6cc(-c7ccc(-c8cccc9ccccc89)cc7)ccc6c45)n3)cc2)cc1 ; c1ccc(-c2ccc(-c3nc(-c4ccccc4)nc(-c4cccc5oc6c(-c7ccc(-c8ccccc8)c8ccccc78)cccc6c45)n3)cc2)cc1]

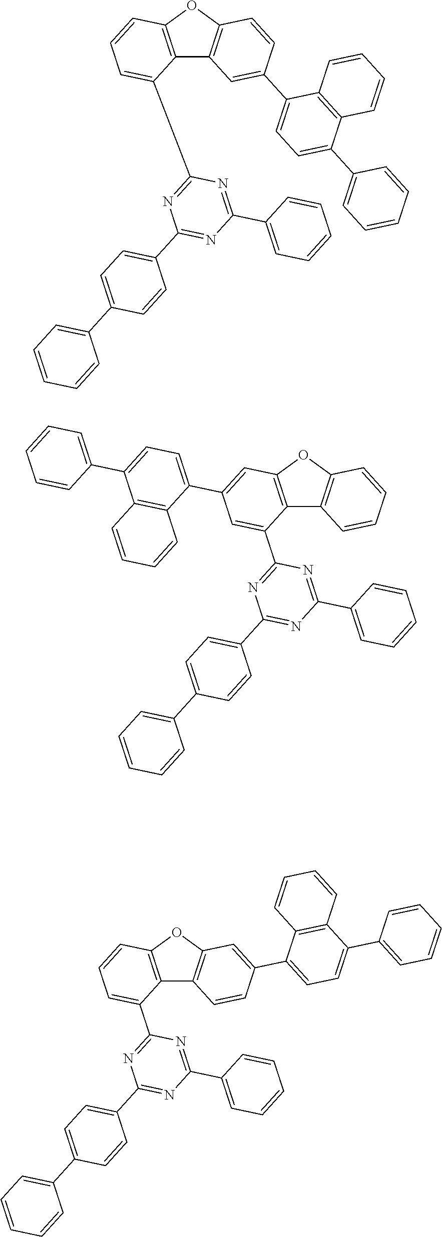

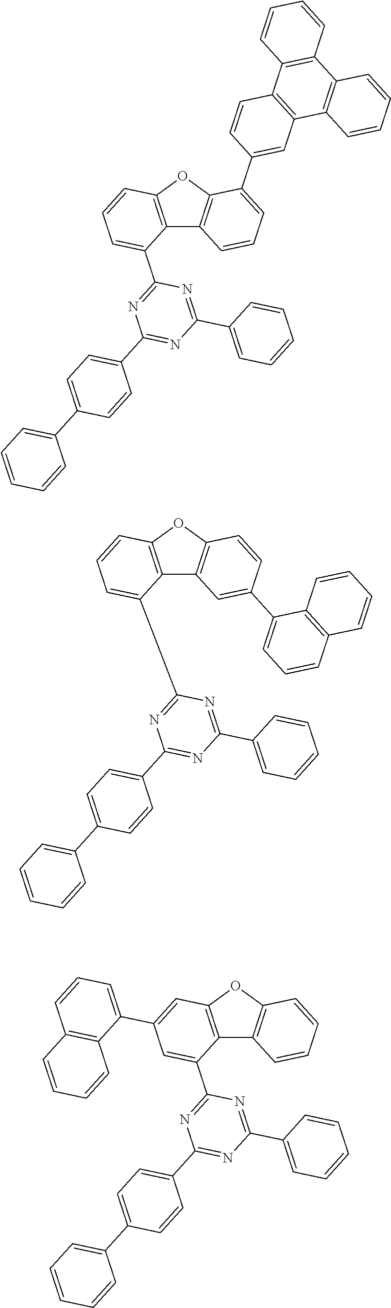 [SMILES: c1ccc(-c2ccc(-c3nc(-c4ccccc4)nc(-c4cccc5oc6c(-c7ccc8c9ccccc9c9ccccc9c8c7)cccc6c45)n3)cc2)cc1 ; c1ccc(-c2ccc(-c3nc(-c4ccccc4)nc(-c4cccc5oc6ccc(-c7cccc8ccccc78)cc6c45)n3)cc2)cc1 ; c1ccc(-c2ccc(-c3nc(-c4ccccc4)nc(-c4cc(-c5cccc6ccccc56)cc5oc6ccccc6c45)n3)cc2)cc1]

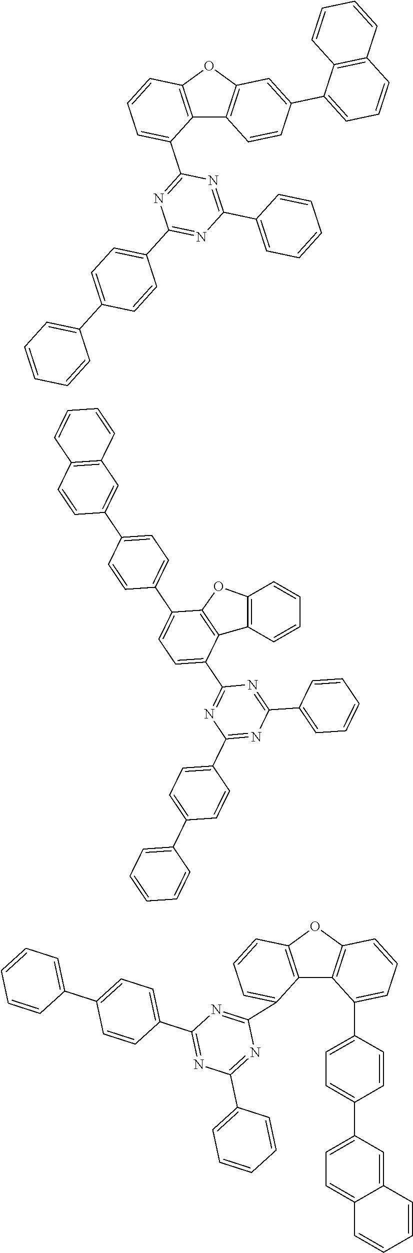

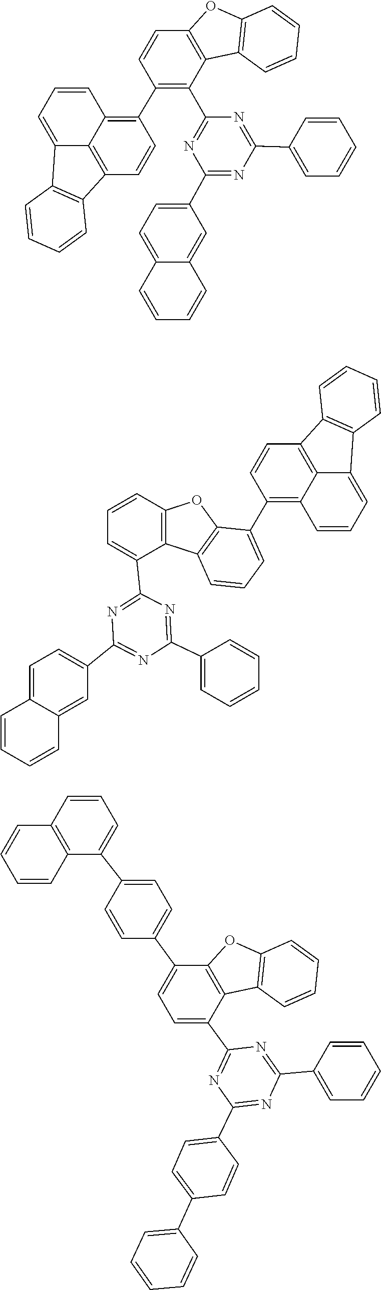 [SMILES: c1ccc(-c2nc(-c3ccc4ccccc4c3)nc(-c3c(-c4ccc5c6c(cccc46)-c4ccccc4-5)ccc4oc5ccccc5c34)n2)cc1 ; c1ccc(-c2nc(-c3ccc4ccccc4c3)nc(-c3cccc4oc5c(-c6ccc7c8c(cccc68)-c6ccccc6-7)cccc5c34)n2)cc1 ; c1ccc(-c2ccc(-c3nc(-c4ccccc4)nc(-c4ccc(-c5ccc(-c6cccc7ccccc67)cc5)c5oc6ccccc6c45)n3)cc2)cc1]

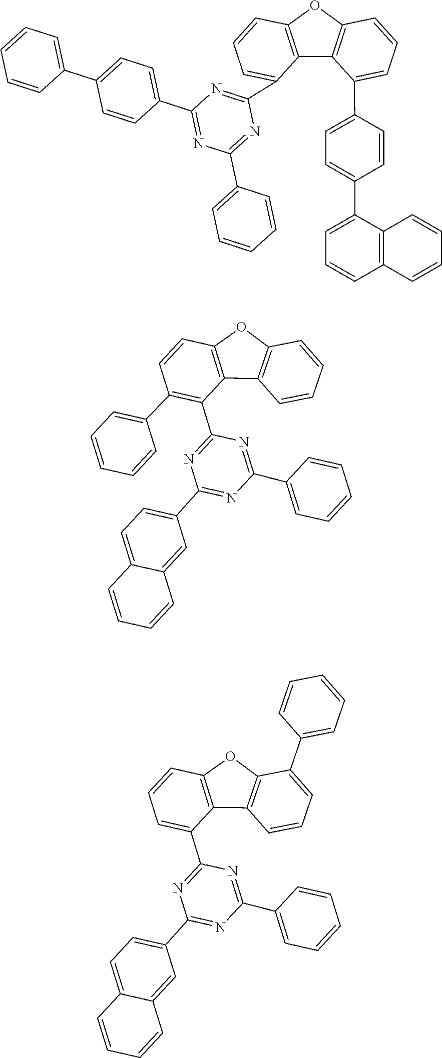 [SMILES: c1ccc(-c2ccc(-c3nc(-c4ccccc4)nc(-c4cccc5oc6cccc(-c7ccc(-c8cccc9ccccc89)cc7)c6c45)n3)cc2)cc1 ; c1ccc(-c2nc(-c3ccc4ccccc4c3)nc(-c3c(-c4ccccc4)ccc4oc5ccccc5c34)n2)cc1 ; c1ccc(-c2nc(-c3ccc4ccccc4c3)nc(-c3cccc4oc5c(-c6ccccc6)cccc5c34)n2)cc1]

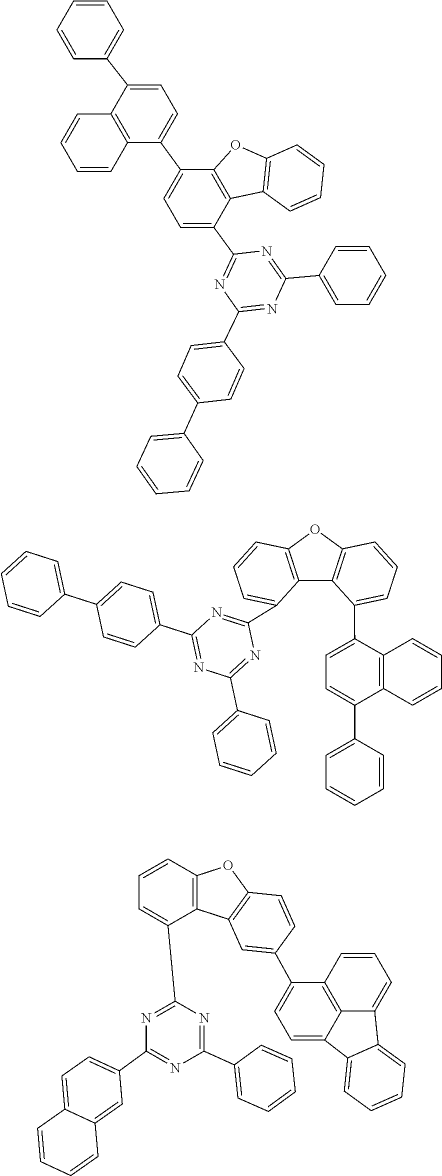 [SMILES: c1ccc(-c2ccc(-c3nc(-c4ccccc4)nc(-c4ccc(-c5ccc(-c6ccccc6)c6ccccc56)c5oc6ccccc6c45)n3)cc2)cc1 ; c1ccc(-c2ccc(-c3nc(-c4ccccc4)nc(-c4cccc5oc6cccc(-c7ccc(-c8ccccc8)c8ccccc78)c6c45)n3)cc2)cc1 ; c1ccc(-c2nc(-c3ccc4ccccc4c3)nc(-c3cccc4oc5ccc(-c6ccc7c8c(cccc68)-c6ccccc6-7)cc5c34)n2)cc1]

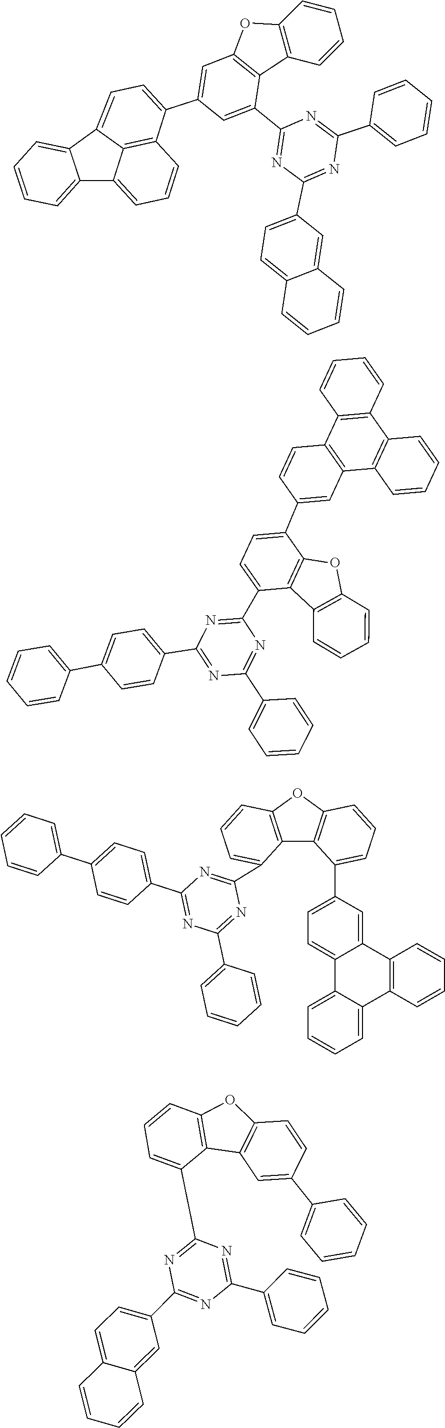

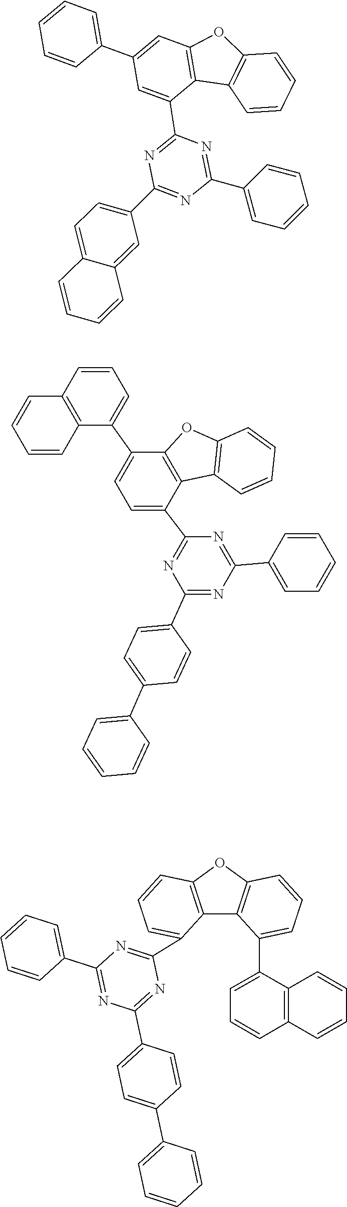

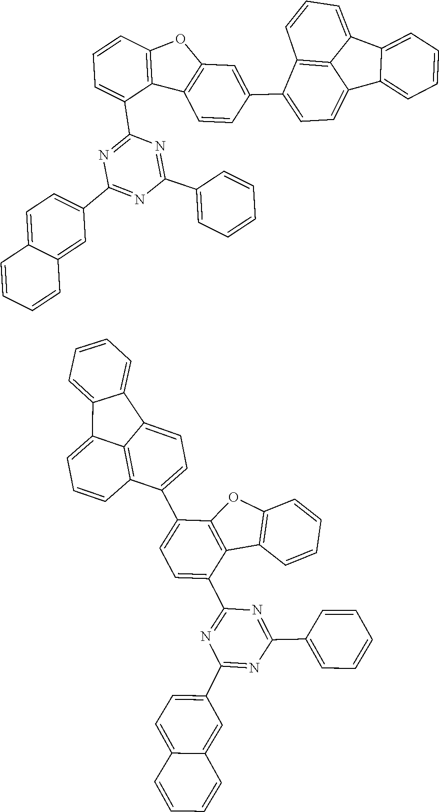 [SMILES: c1ccc(-c2nc(-c3ccc4ccccc4c3)nc(-c3cccc4oc5cc(-c6ccc7c8c(cccc68)-c6ccccc6-7)ccc5c34)n2)cc1 ; c1ccc(-c2nc(-c3ccc4ccccc4c3)nc(-c3ccc(-c4ccc5c6c(cccc46)-c4ccccc4-5)c4oc5ccccc5c34)n2)cc1]

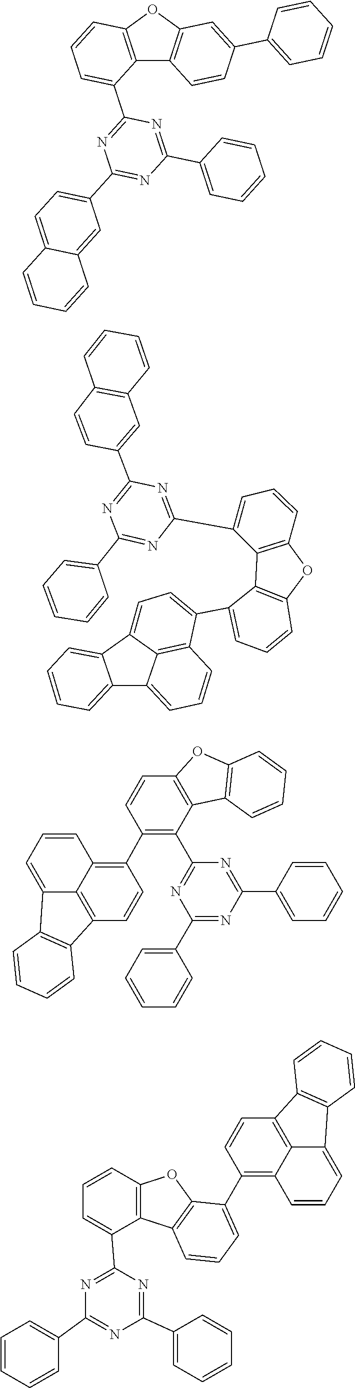

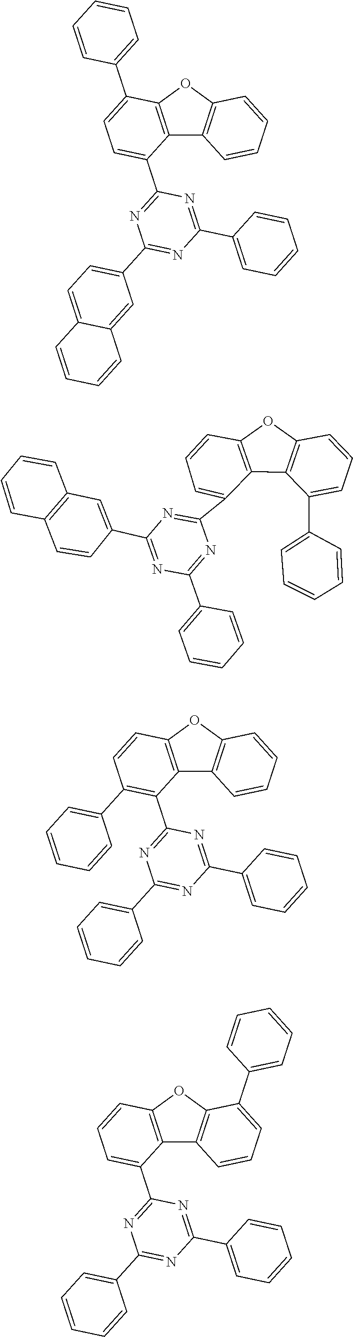 [SMILES: c1ccc(-c2nc(-c3ccc4ccccc4c3)nc(-c3ccc(-c4ccccc4)c4oc5ccccc5c34)n2)cc1 ; c1ccc(-c2nc(-c3ccc4ccccc4c3)nc(-c3cccc4oc5cccc(-c6ccccc6)c5c34)n2)cc1 ; c1ccc(-c2nc(-c3ccccc3)nc(-c3c(-c4ccccc4)ccc4oc5ccccc5c34)n2)cc1 ; c1ccc(-c2nc(-c3ccccc3)nc(-c3cccc4oc5c(-c6ccccc6)cccc5c34)n2)cc1]

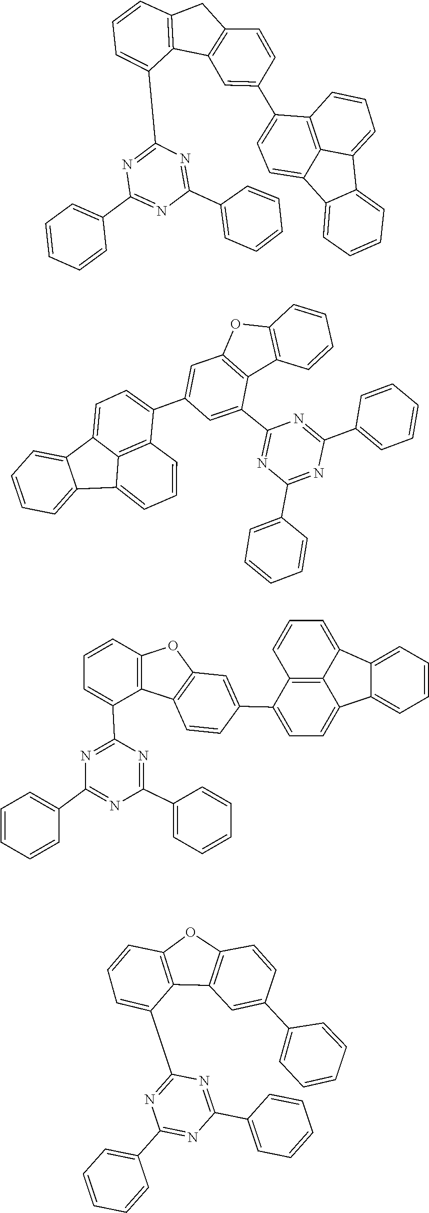

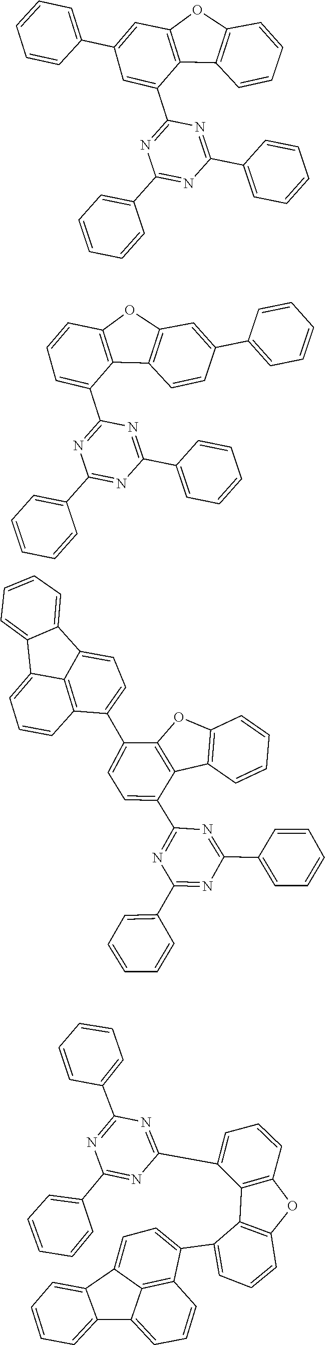

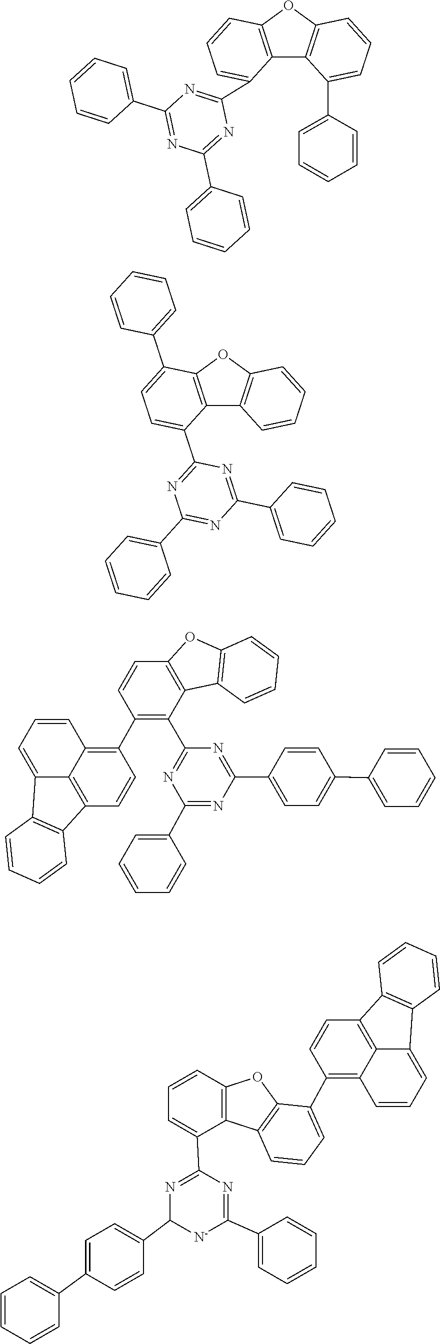

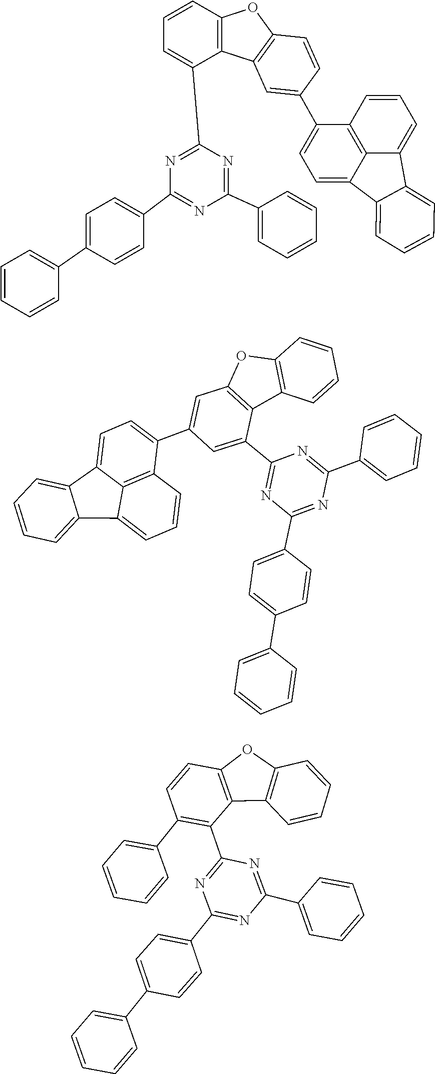

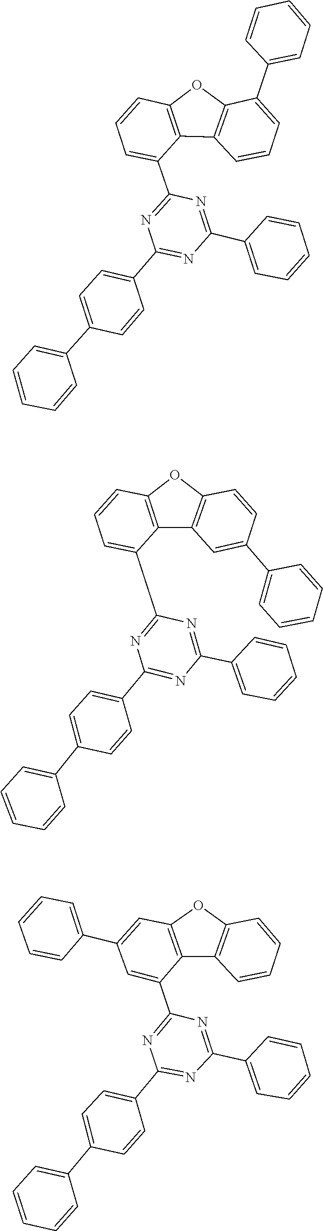 [SMILES: c1ccc(-c2ccc(-c3nc(-c4ccccc4)nc(-c4cccc5oc6c(-c7ccccc7)cccc6c45)n3)cc2)cc1 ; c1ccc(-c2ccc(-c3nc(-c4ccccc4)nc(-c4cccc5oc6ccc(-c7ccccc7)cc6c45)n3)cc2)cc1 ; c1ccc(-c2ccc(-c3nc(-c4ccccc4)nc(-c4cc(-c5ccccc5)cc5oc6ccccc6c45)n3)cc2)cc1]

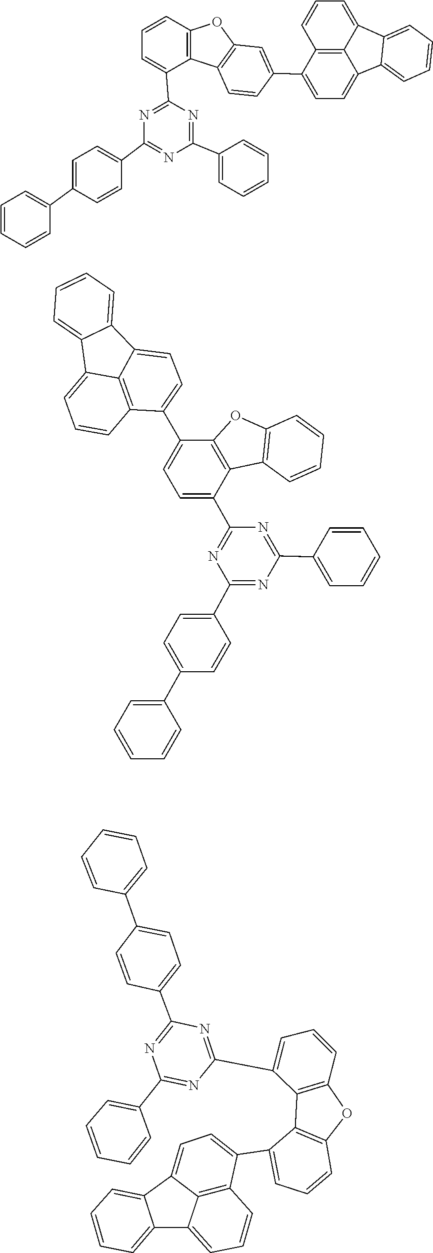 [SMILES: c1ccc(-c2ccc(-c3nc(-c4ccccc4)nc(-c4cccc5oc6cc(-c7ccc8c9c(cccc79)-c7ccccc7-8)ccc6c45)n3)cc2)cc1 ; c1ccc(-c2ccc(-c3nc(-c4ccccc4)nc(-c4ccc(-c5ccc6c7c(cccc57)-c5ccccc5-6)c5oc6ccccc6c45)n3)cc2)cc1 ; c1ccc(-c2ccc(-c3nc(-c4ccccc4)nc(-c4cccc5oc6cccc(-c7ccc8c9c(cccc79)-c7ccccc7-8)c6c45)n3)cc2)cc1]

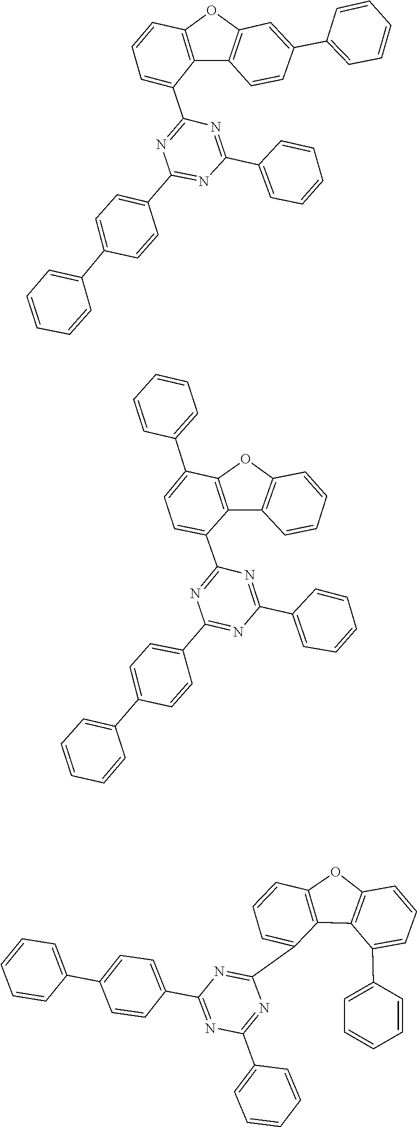 [SMILES: c1ccc(-c2ccc(-c3nc(-c4ccccc4)nc(-c4cccc5oc6cc(-c7ccccc7)ccc6c45)n3)cc2)cc1 ; c1ccc(-c2ccc(-c3nc(-c4ccccc4)nc(-c4ccc(-c5ccccc5)c5oc6ccccc6c45)n3)cc2)cc1 ; c1ccc(-c2ccc(-c3nc(-c4ccccc4)nc(-c4cccc5oc6cccc(-c7ccccc7)c6c45)n3)cc2)cc1]

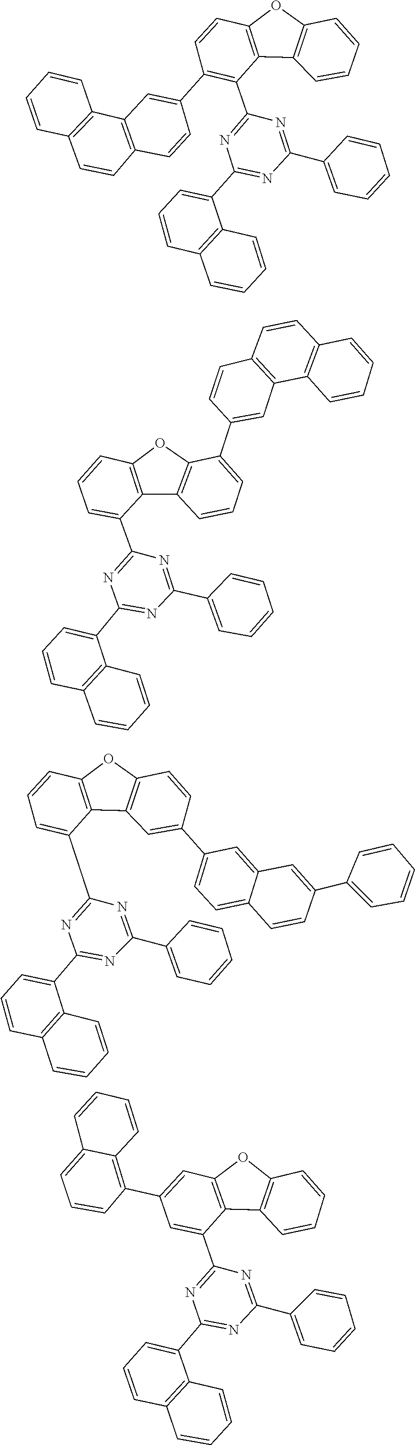

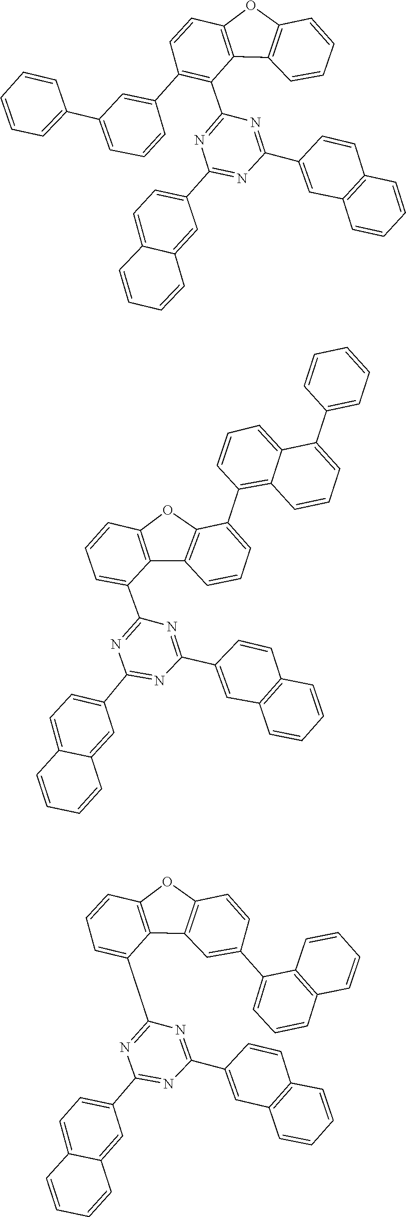 [SMILES: c1ccc(-c2cccc(-c3ccc4oc5ccccc5c4c3-c3nc(-c4ccc5ccccc5c4)nc(-c4ccc5ccccc5c4)n3)c2)cc1 ; c1ccc(-c2cccc3c(-c4cccc5c4oc4cccc(-c6nc(-c7ccc8ccccc8c7)nc(-c7ccc8ccccc8c7)n6)c45)cccc23)cc1 ; c1ccc2cc(-c3nc(-c4ccc5ccccc5c4)nc(-c4cccc5oc6ccc(-c7cccc8ccccc78)cc6c45)n3)ccc2c1]

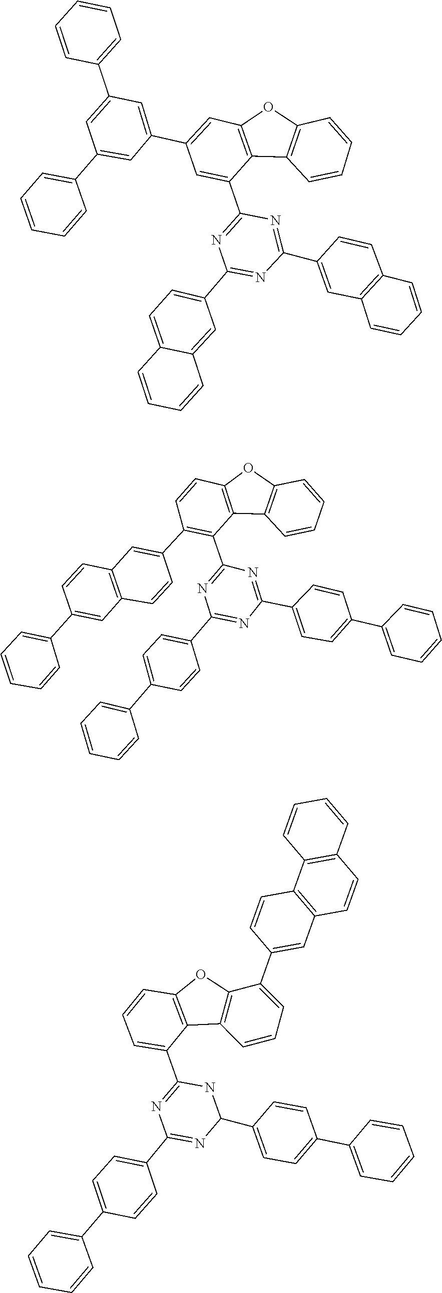

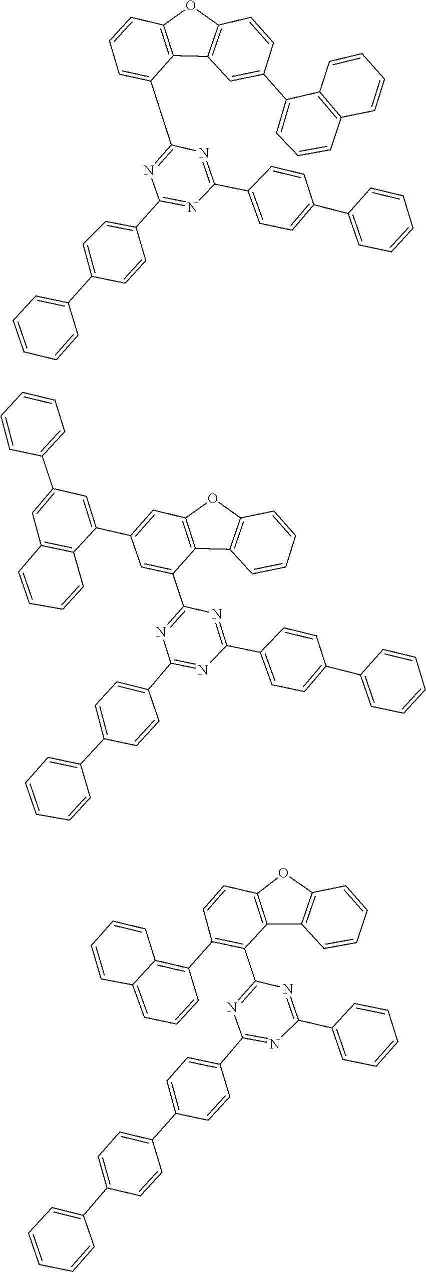

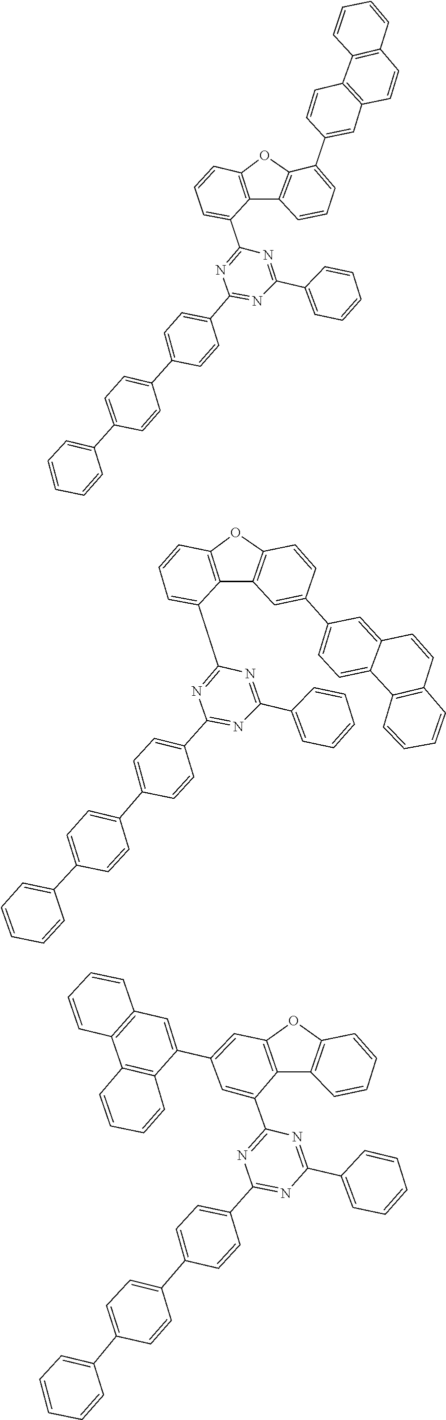 [SMILES: c1ccc(-c2ccc(-c3ccc(-c4nc(-c5ccccc5)nc(-c5cccc6oc7c(-c8ccc9c(ccc%10ccccc%109)c8)cccc7c56)n4)cc3)cc2)cc1 ; c1ccc(-c2ccc(-c3ccc(-c4nc(-c5ccccc5)nc(-c5cccc6oc7ccc(-c8ccc9c(ccc%10ccccc%109)c8)cc7c56)n4)cc3)cc2)cc1 ; c1ccc(-c2ccc(-c3ccc(-c4nc(-c5ccccc5)nc(-c5cc(-c6cc7ccccc7c7ccccc67)cc6oc7ccccc7c56)n4)cc3)cc2)cc1]

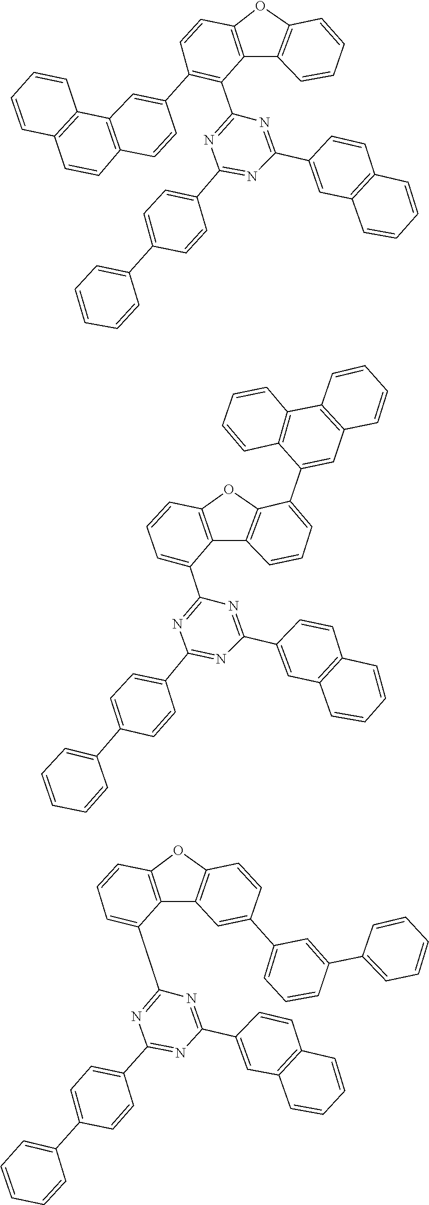 [SMILES: c1ccc(-c2ccc(-c3nc(-c4ccc5ccccc5c4)nc(-c4c(-c5ccc6ccc7ccccc7c6c5)ccc5oc6ccccc6c45)n3)cc2)cc1 ; c1ccc(-c2ccc(-c3nc(-c4ccc5ccccc5c4)nc(-c4cccc5oc6c(-c7cc8ccccc8c8ccccc78)cccc6c45)n3)cc2)cc1 ; c1ccc(-c2ccc(-c3nc(-c4ccc5ccccc5c4)nc(-c4cccc5oc6ccc(-c7cccc(-c8ccccc8)c7)cc6c45)n3)cc2)cc1]

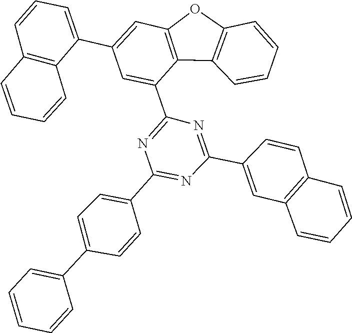 [SMILES: c1ccc(-c2ccc(-c3nc(-c4ccc5ccccc5c4)nc(-c4cc(-c5cccc6ccccc56)cc5oc6ccccc6c45)n3)cc2)cc1]

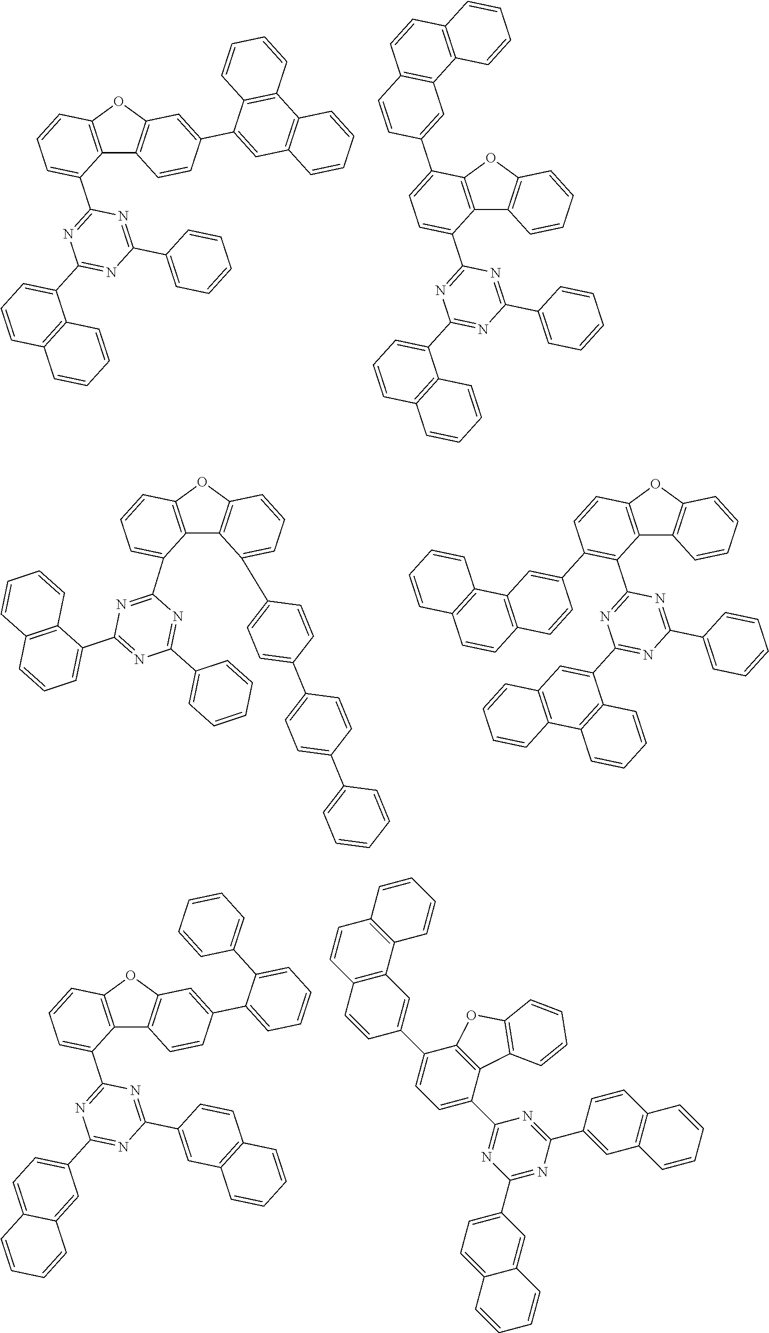

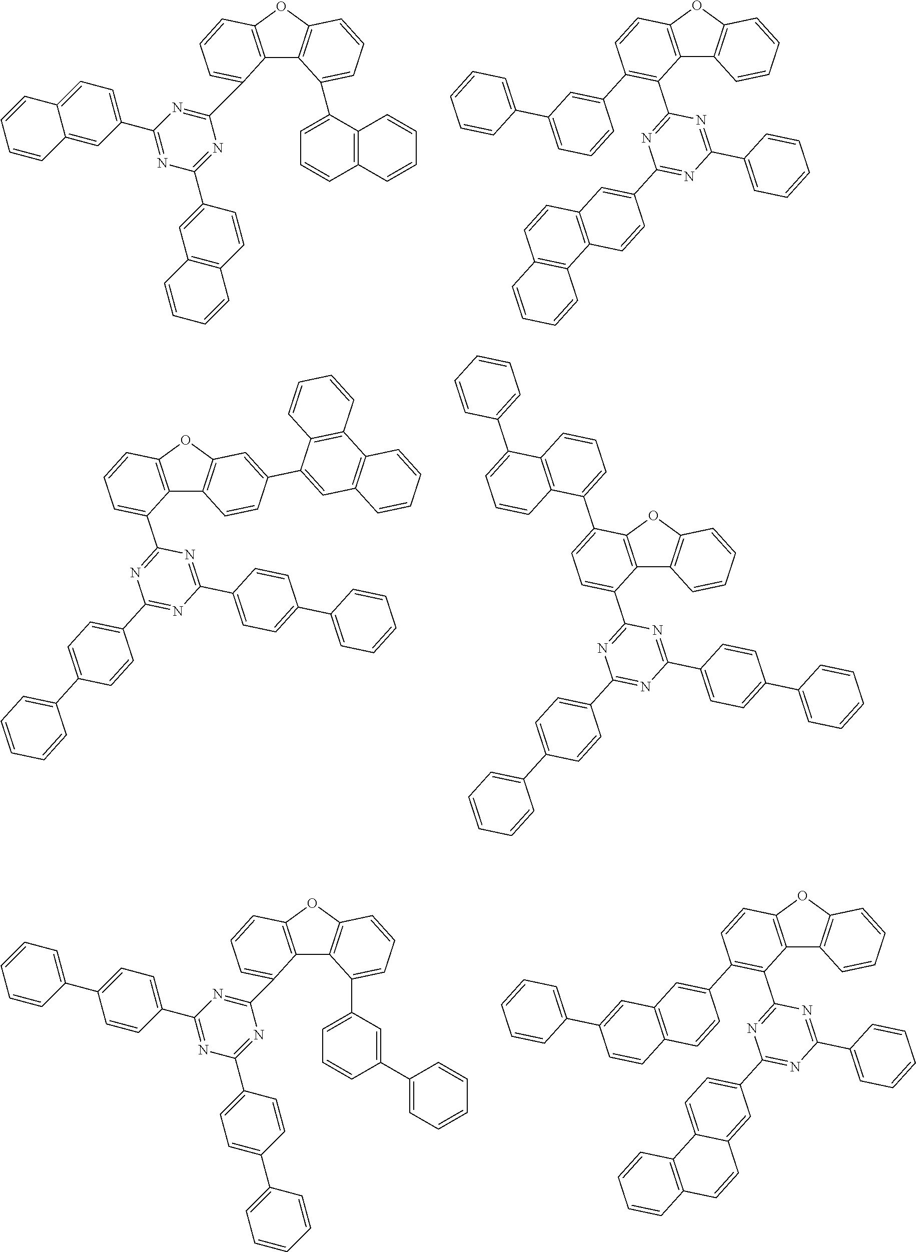

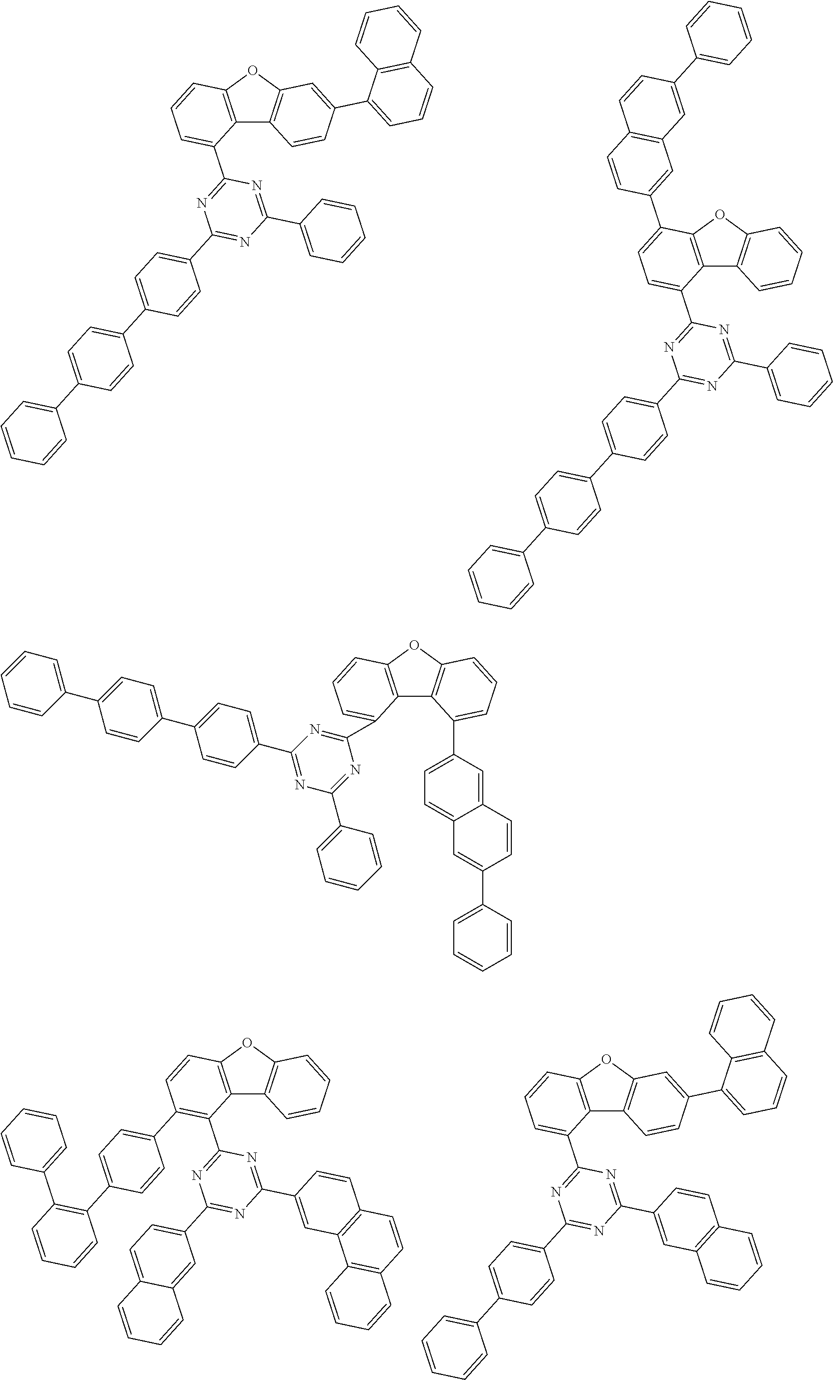 [SMILES: c1ccc(-c2ccc(-c3ccc(-c4nc(-c5ccccc5)nc(-c5cccc6oc7cc(-c8cccc9ccccc89)ccc7c56)n4)cc3)cc2)cc1 ; c1ccc(-c2ccc(-c3ccc(-c4nc(-c5ccccc5)nc(-c5ccc(-c6ccc7ccc(-c8ccccc8)cc7c6)c6oc7ccccc7c56)n4)cc3)cc2)cc1 ; c1ccc(-c2ccc(-c3ccc(-c4nc(-c5ccccc5)nc(-c5cccc6oc7cccc(-c8ccc9cc(-c%10ccccc%10)ccc9c8)c7c56)n4)cc3)cc2)cc1 ; c1ccc(-c2ccccc2-c2ccc(-c3ccc4oc5ccccc5c4c3-c3nc(-c4ccc5ccccc5c4)nc(-c4ccc5ccc6ccccc6c5c4)n3)cc2)cc1 ; c1ccc(-c2ccc(-c3nc(-c4ccc5ccccc5c4)nc(-c4cccc5oc6cc(-c7cccc8ccccc78)ccc6c45)n3)cc2)cc1]

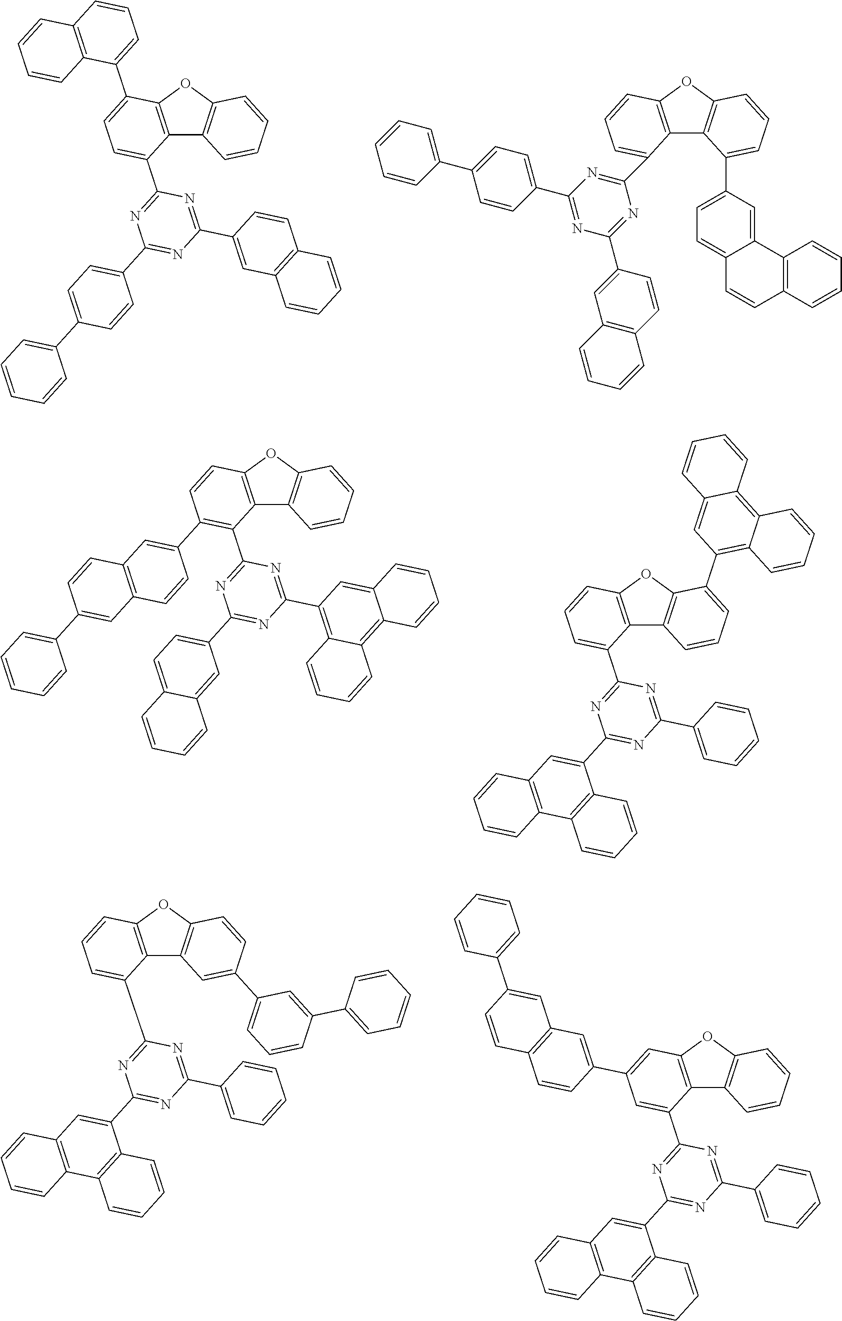

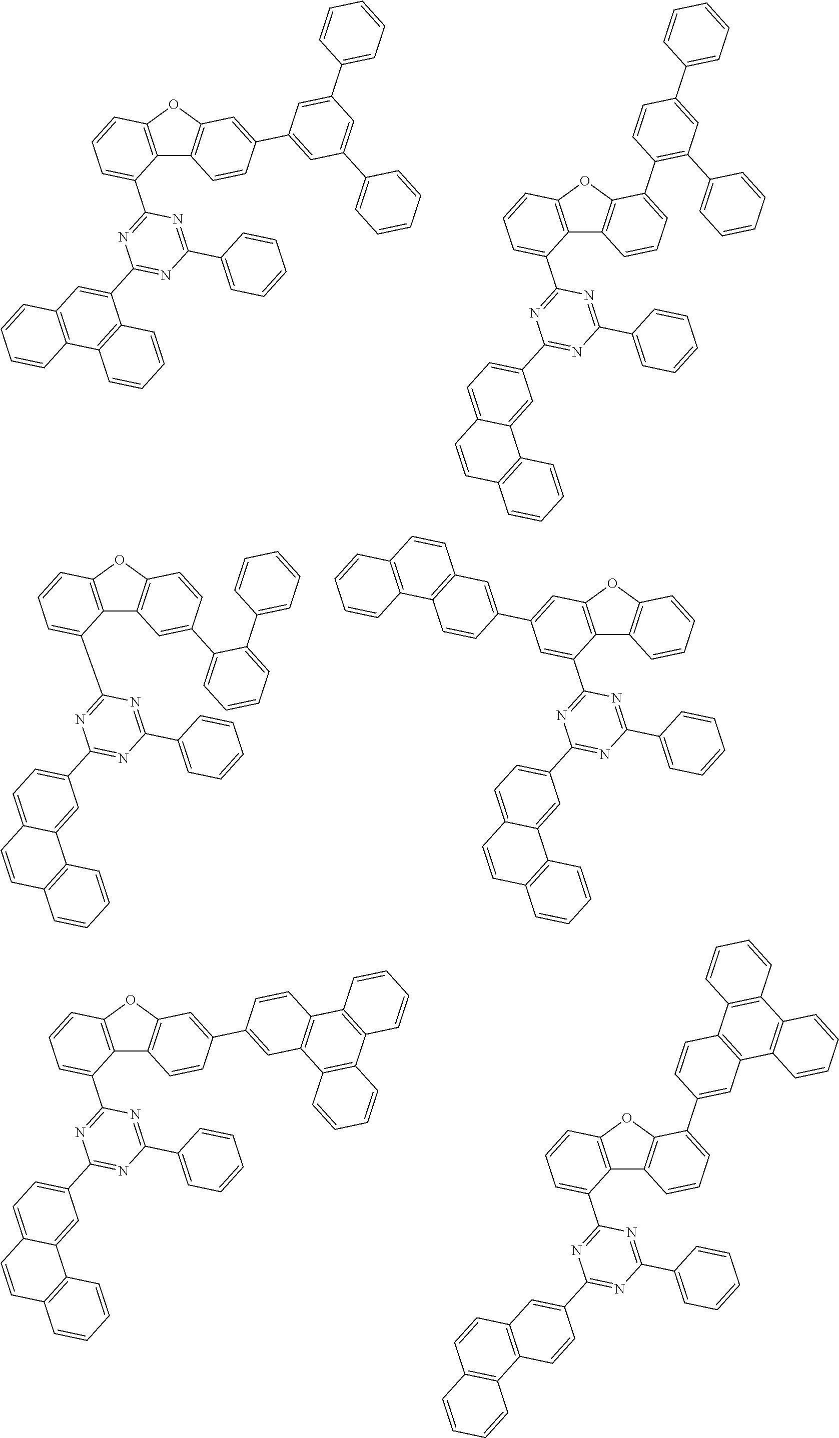

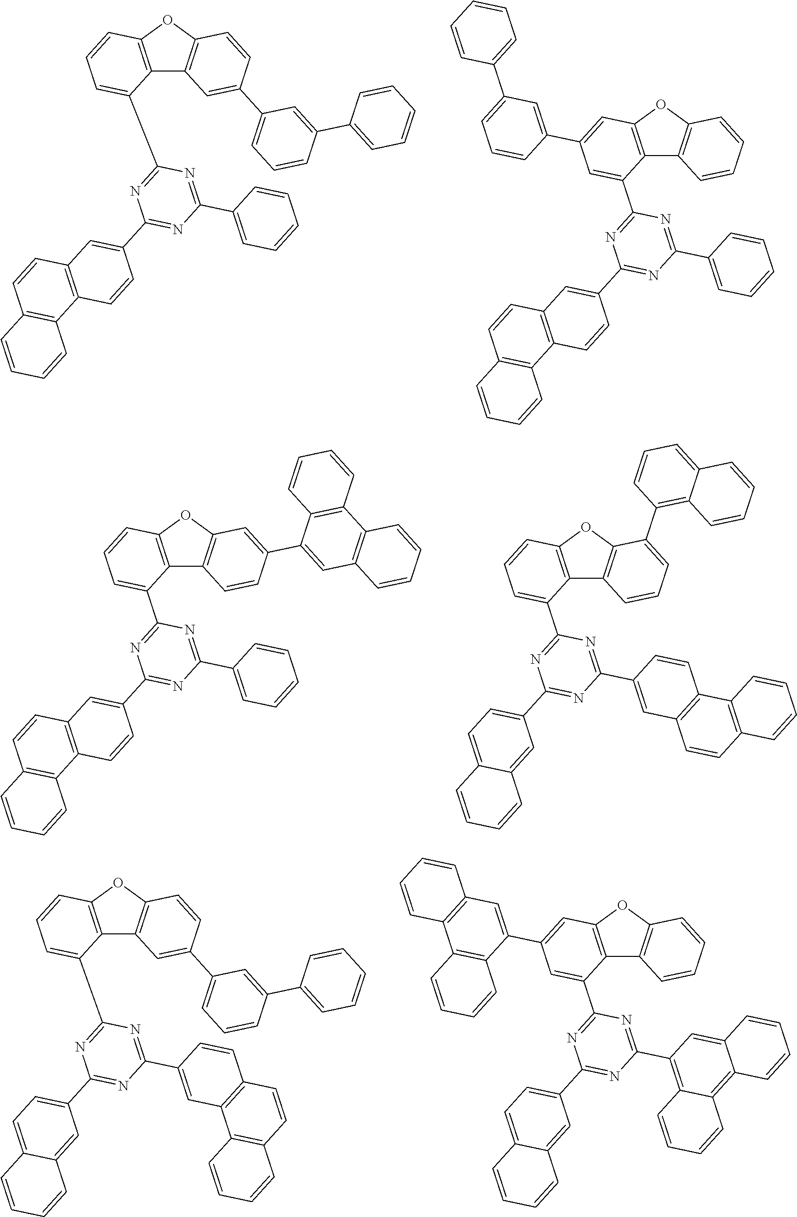

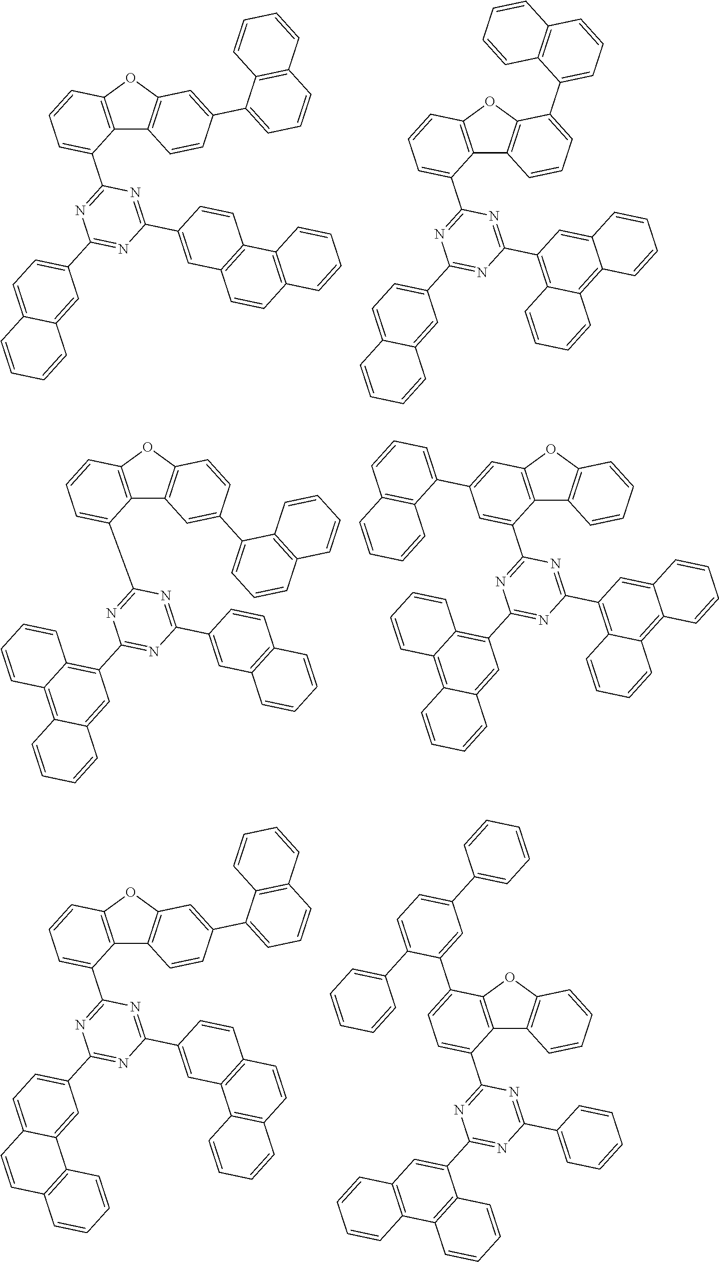

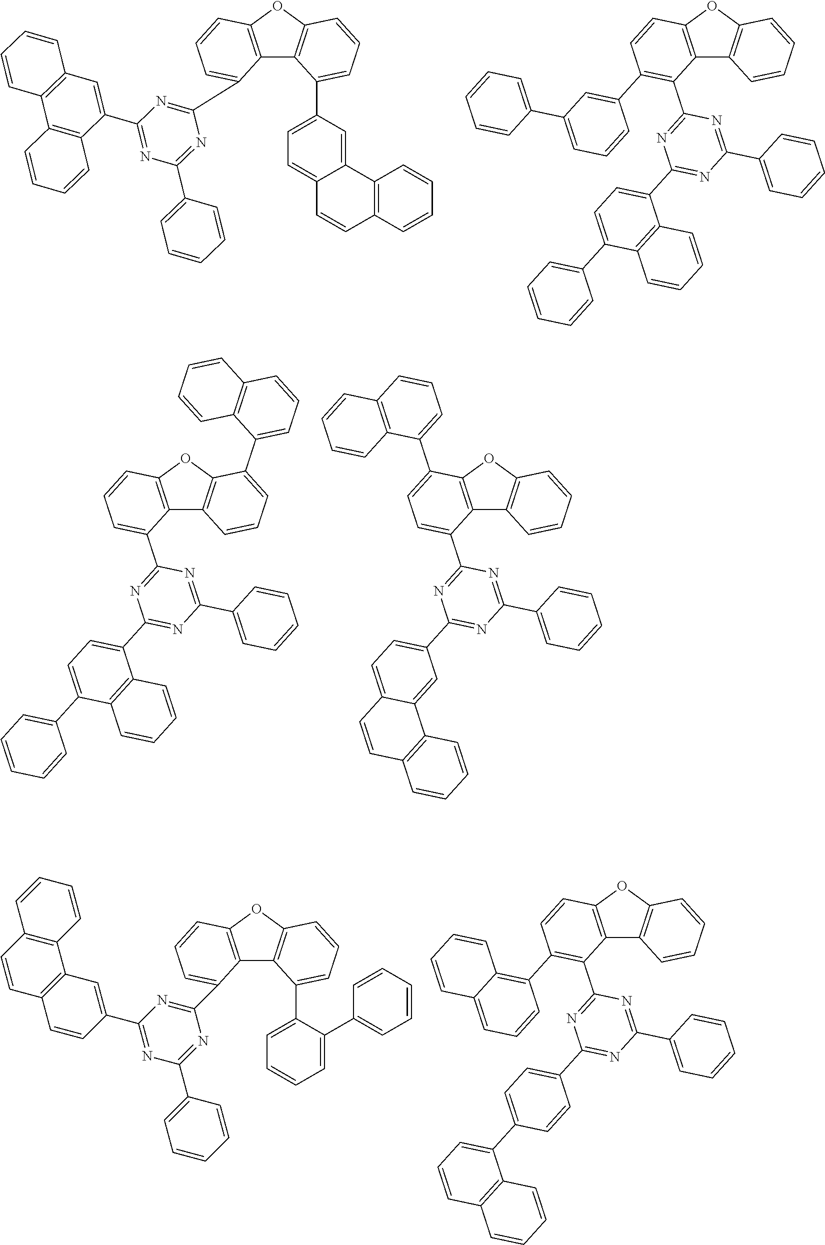

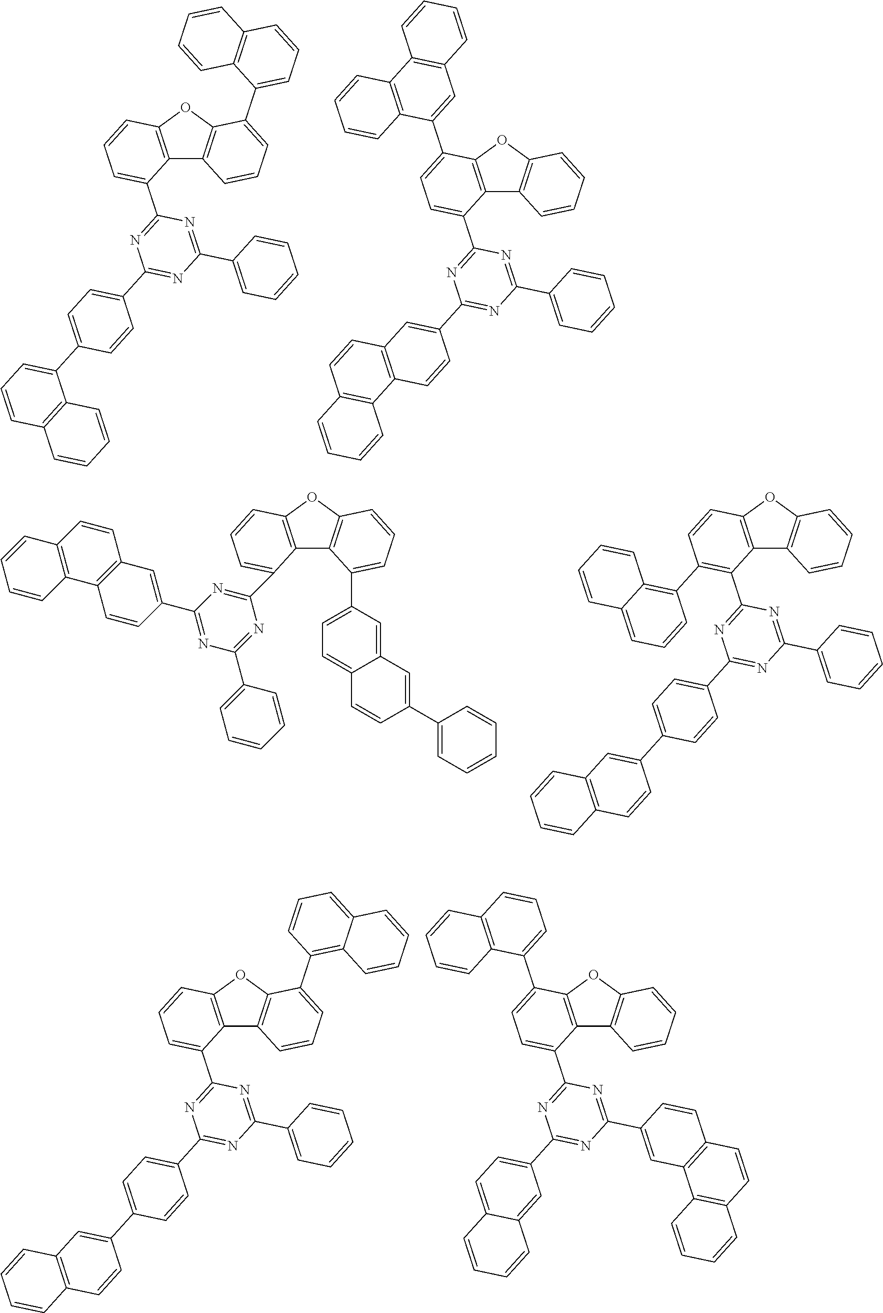

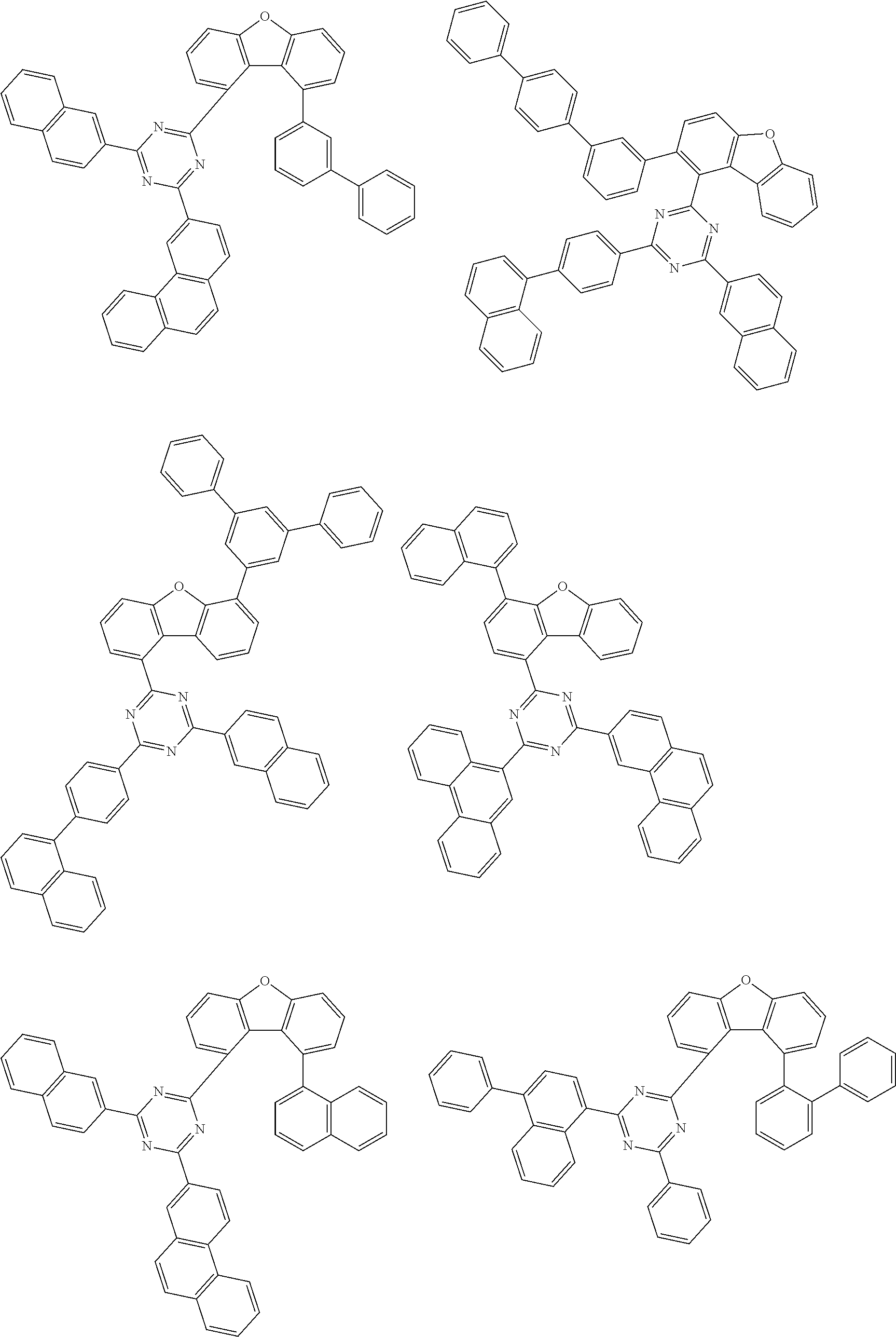

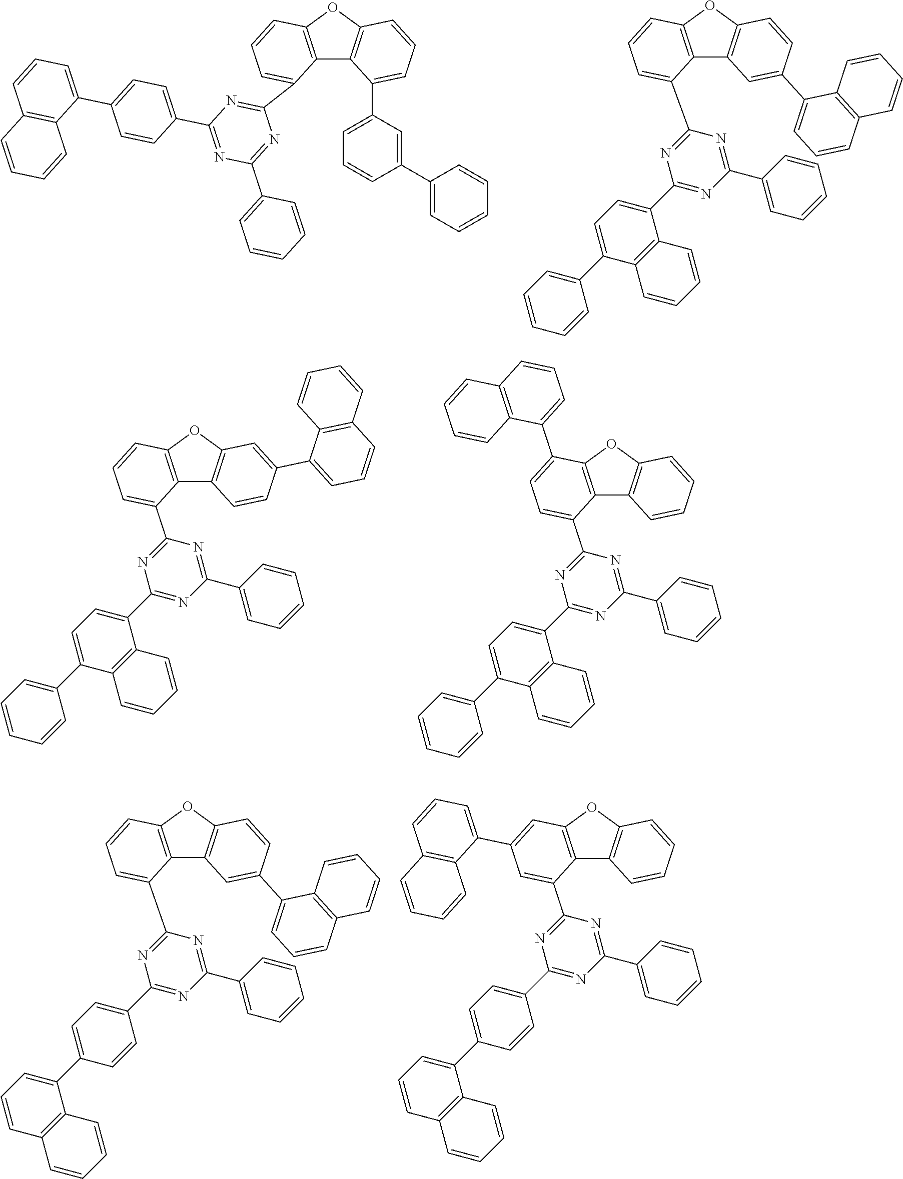

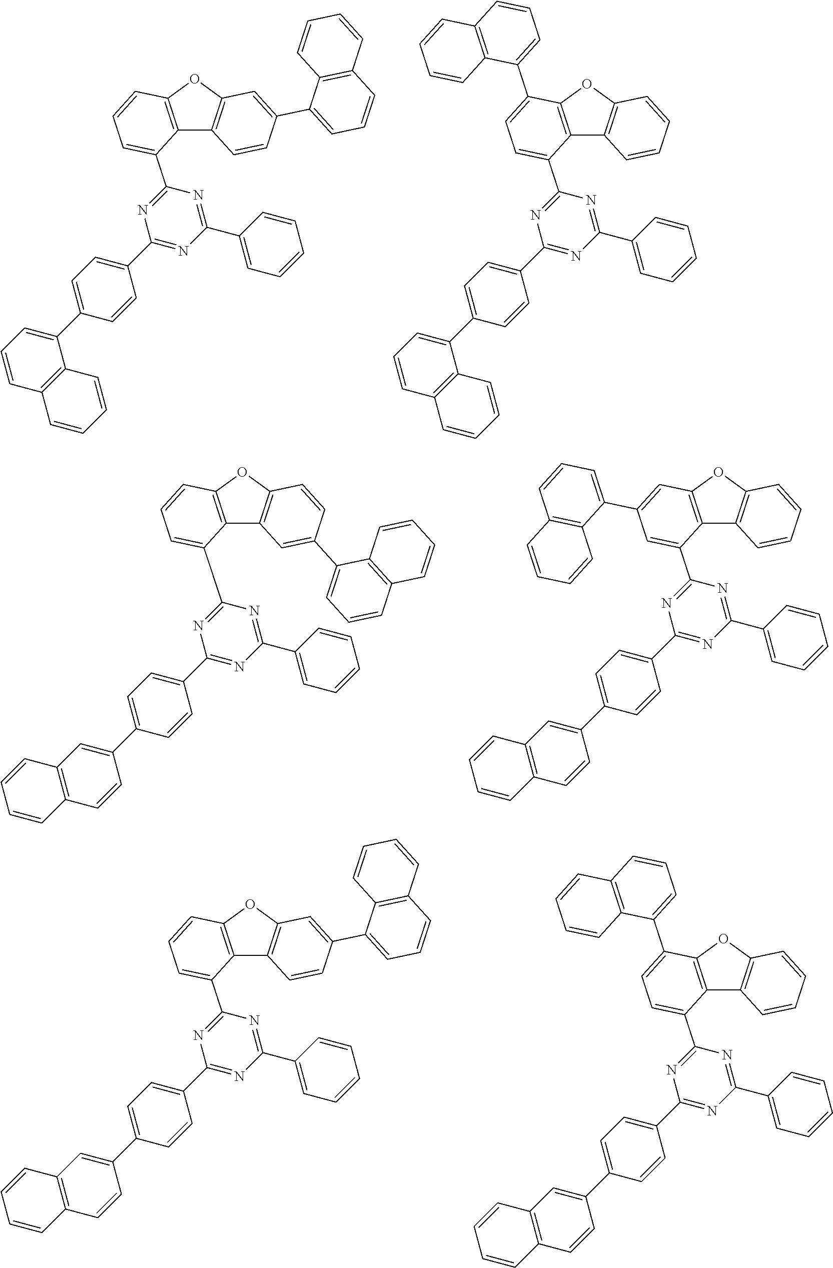

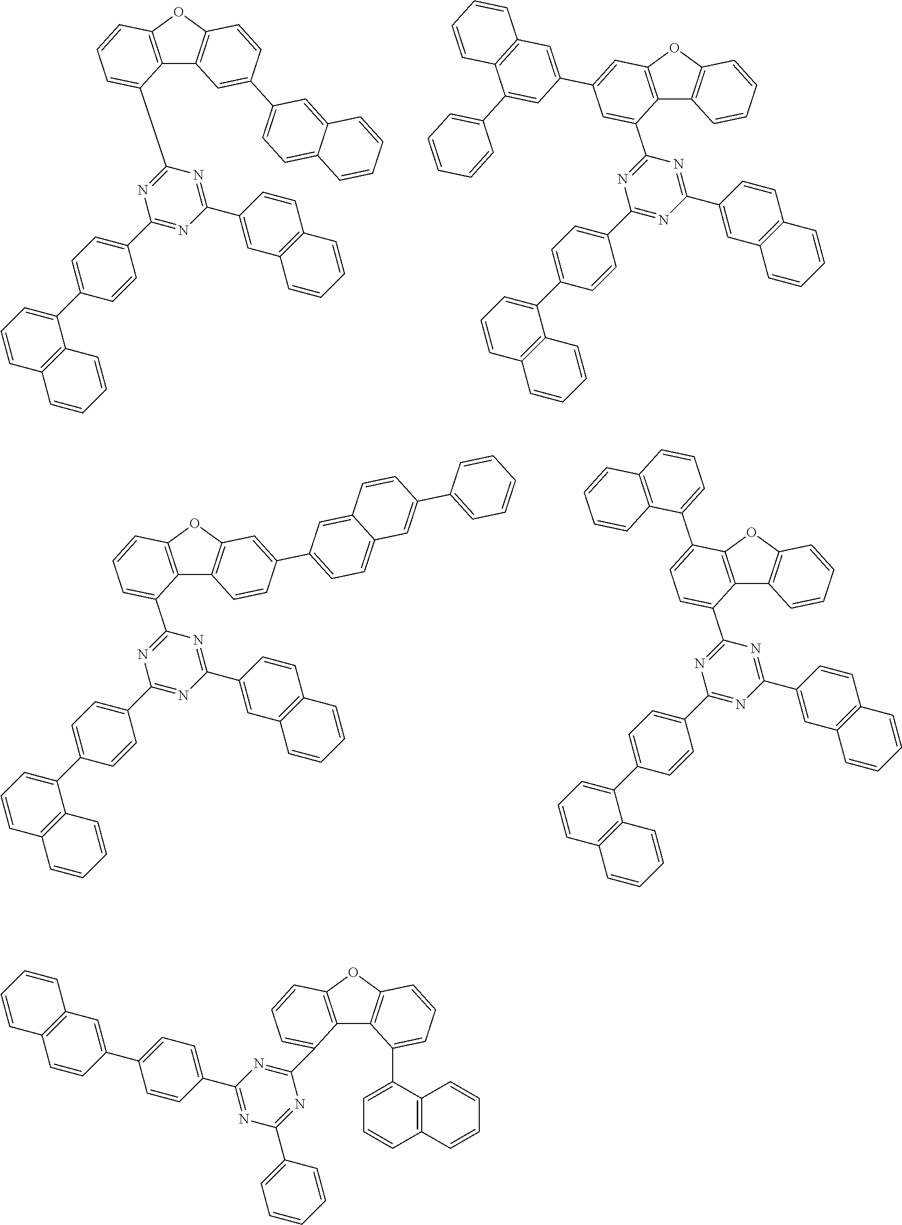

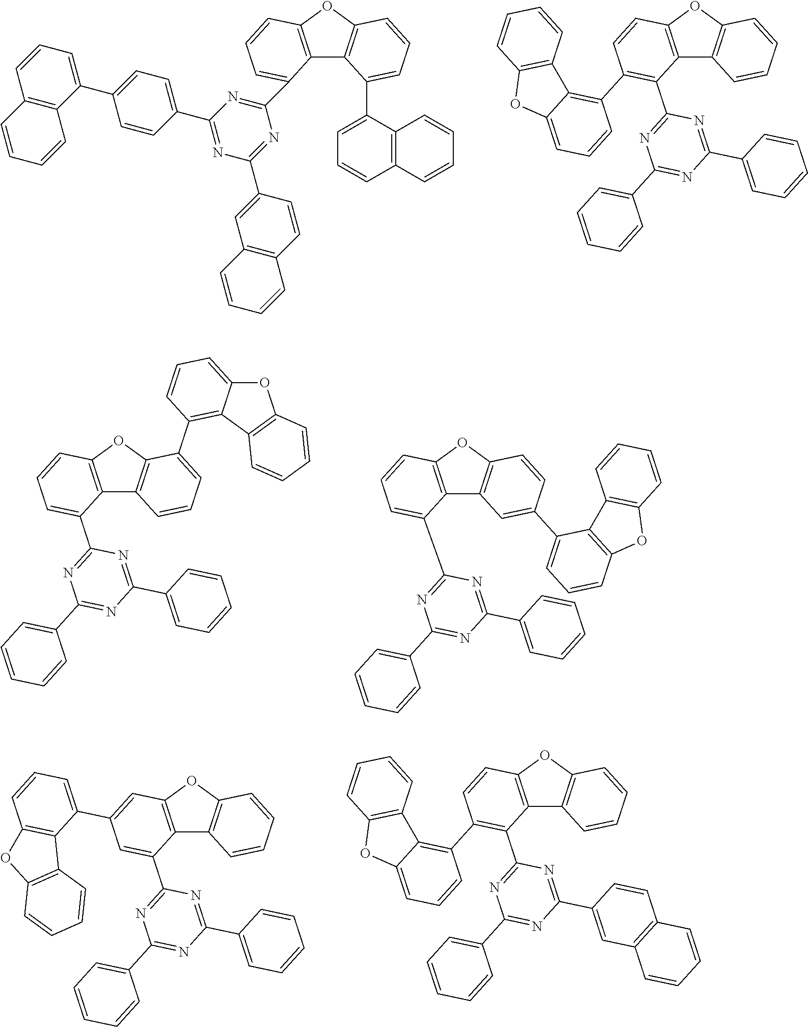

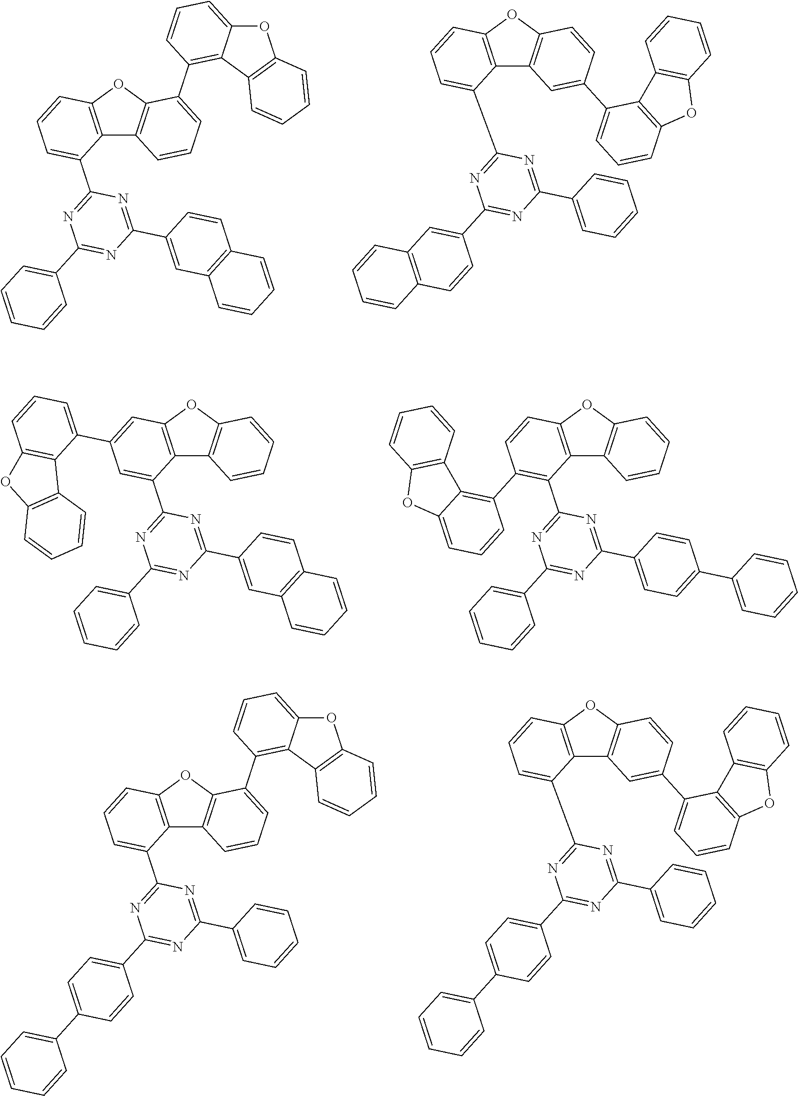

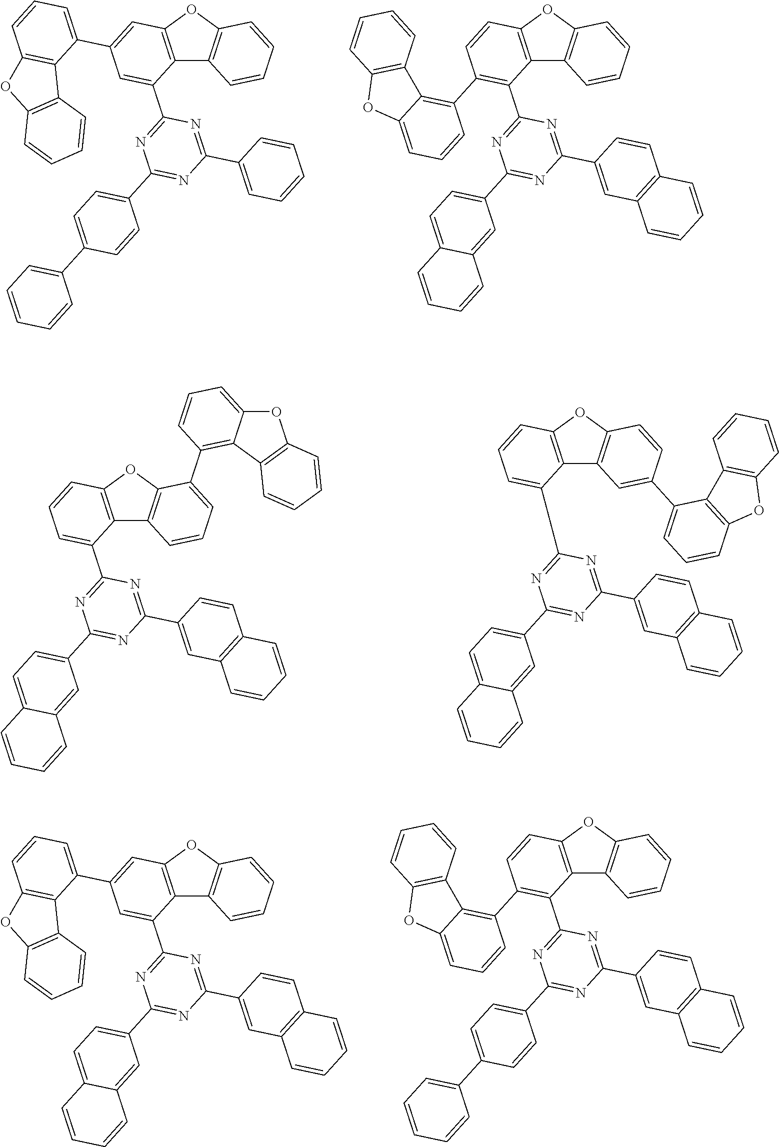

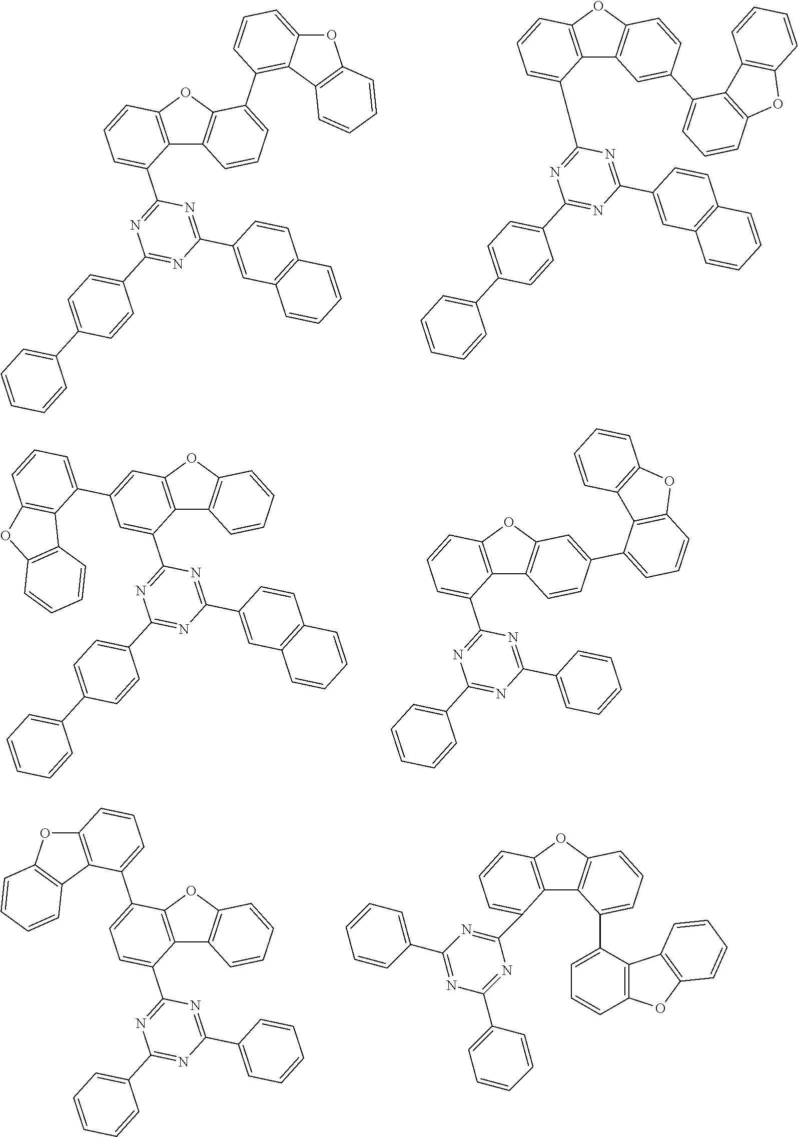

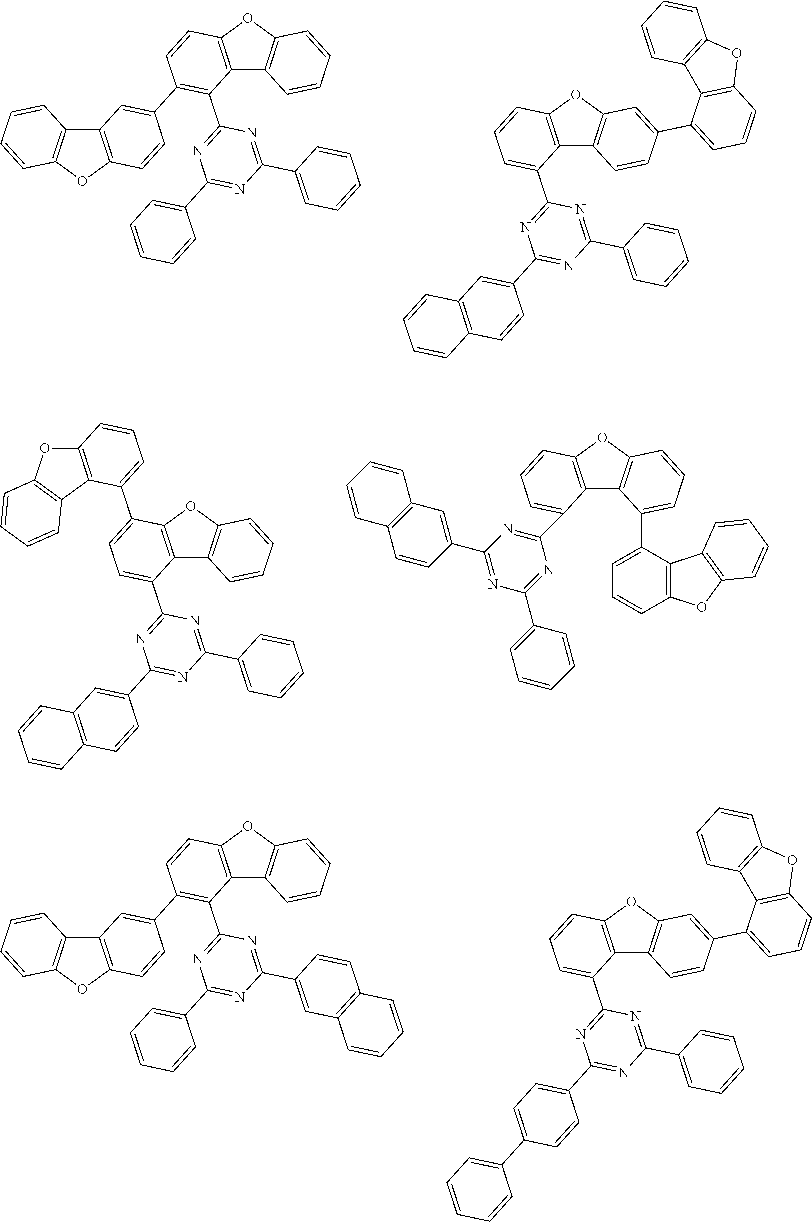 [SMILES: c1ccc(-c2nc(-c3ccccc3)nc(-c3c(-c4ccc5oc6ccccc6c5c4)ccc4oc5ccccc5c34)n2)cc1 ; c1ccc(-c2nc(-c3ccc4ccccc4c3)nc(-c3cccc4oc5cc(-c6cccc7oc8ccccc8c67)ccc5c34)n2)cc1 ; c1ccc(-c2nc(-c3ccc4ccccc4c3)nc(-c3ccc(-c4cccc5oc6ccccc6c45)c4oc5ccccc5c34)n2)cc1 ; c1ccc(-c2nc(-c3ccc4ccccc4c3)nc(-c3cccc4oc5cccc(-c6cccc7oc8ccccc8c67)c5c34)n2)cc1 ; c1ccc(-c2nc(-c3ccc4ccccc4c3)nc(-c3c(-c4ccc5oc6ccccc6c5c4)ccc4oc5ccccc5c34)n2)cc1 ; c1ccc(-c2ccc(-c3nc(-c4ccccc4)nc(-c4cccc5oc6cc(-c7cccc8oc9ccccc9c78)ccc6c45)n3)cc2)cc1]

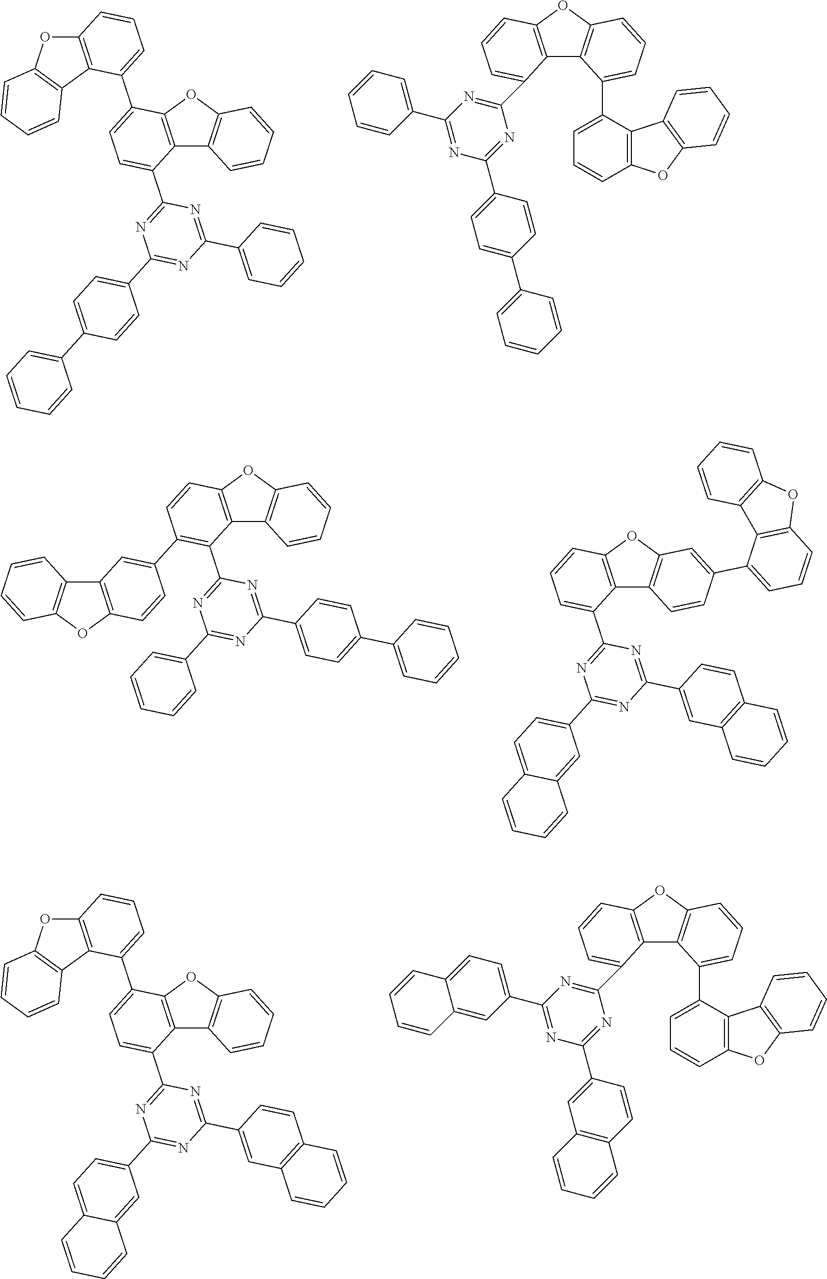

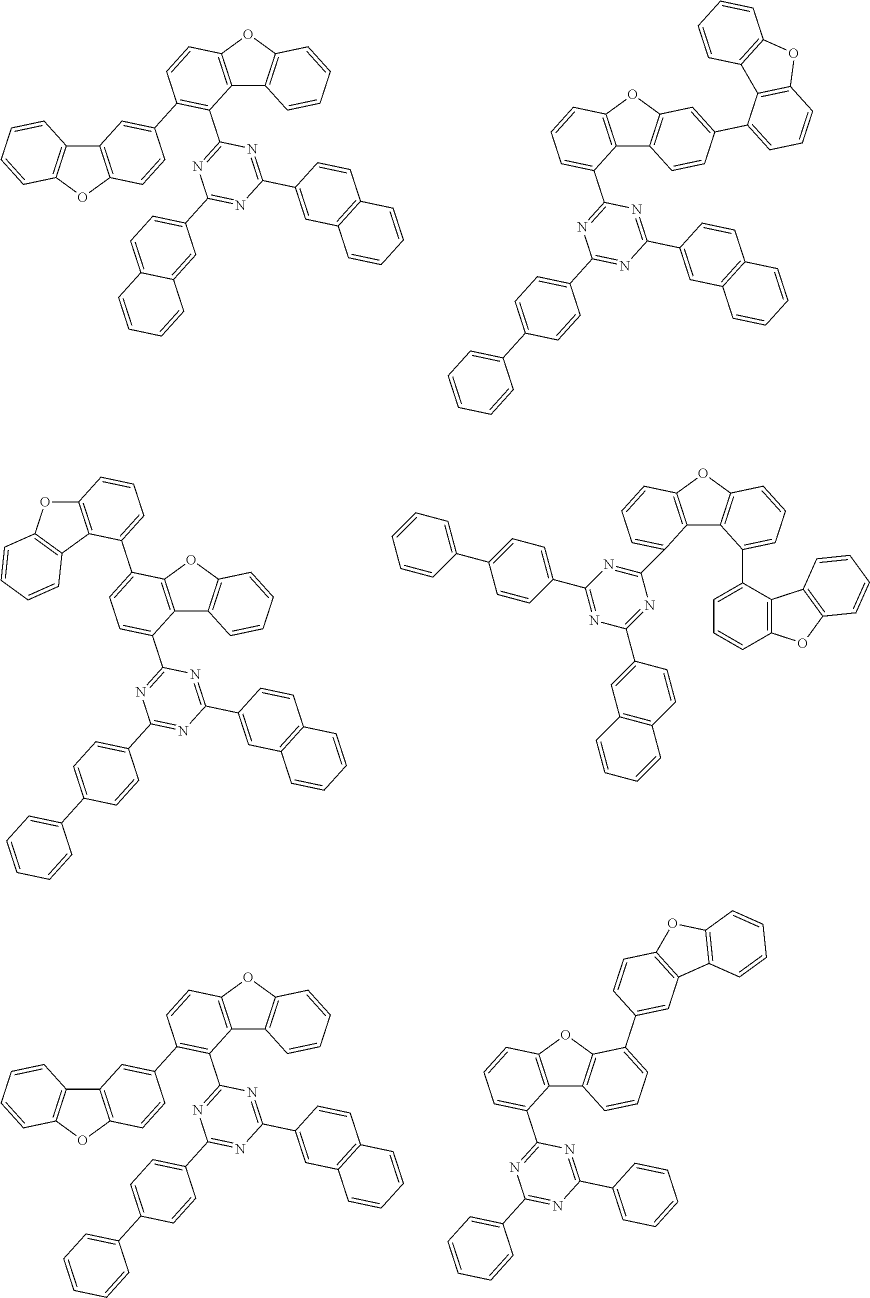

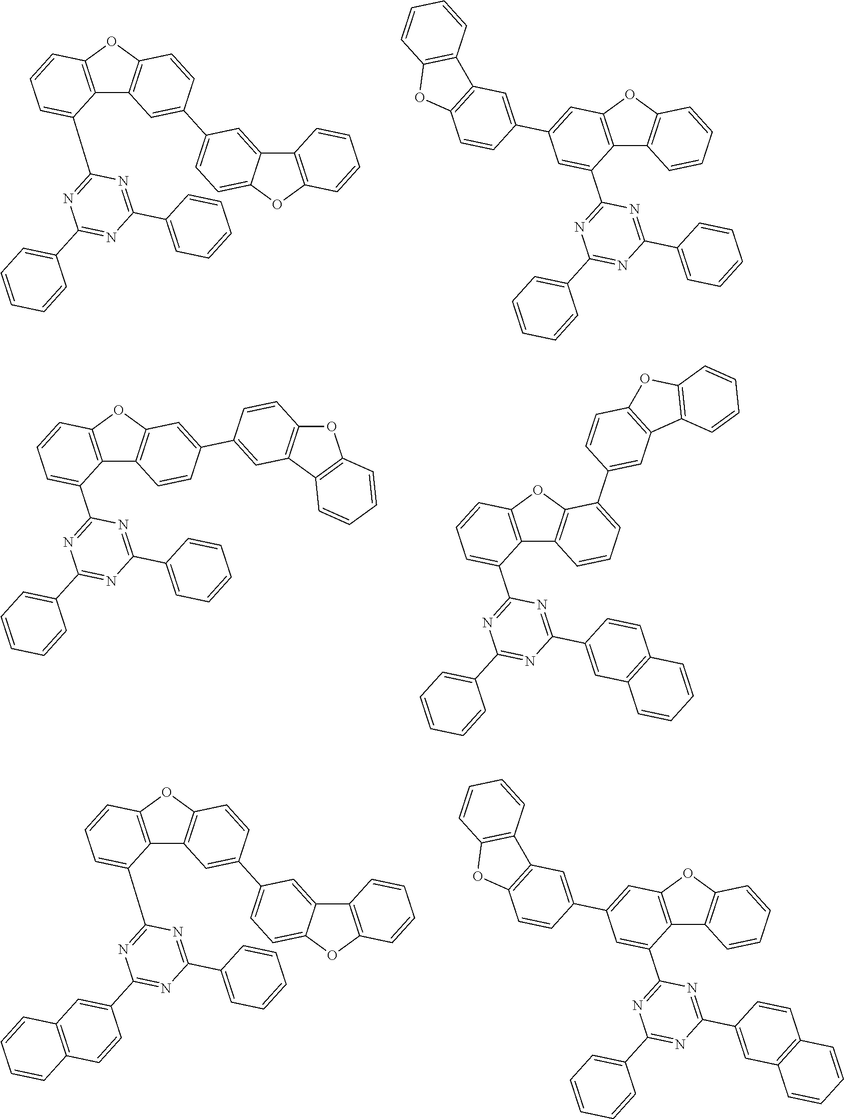 [SMILES: c1ccc(-c2nc(-c3ccccc3)nc(-c3cccc4oc5ccc(-c6ccc7oc8ccccc8c7c6)cc5c34)n2)cc1 ; c1ccc(-c2nc(-c3ccccc3)nc(-c3cc(-c4ccc5oc6ccccc6c5c4)cc4oc5ccccc5c34)n2)cc1 ; c1ccc(-c2nc(-c3ccccc3)nc(-c3cccc4oc5cc(-c6ccc7oc8ccccc8c7c6)ccc5c34)n2)cc1 ; c1ccc(-c2nc(-c3ccc4ccccc4c3)nc(-c3cccc4oc5c(-c6ccc7oc8ccccc8c7c6)cccc5c34)n2)cc1 ; c1ccc(-c2nc(-c3ccc4ccccc4c3)nc(-c3cccc4oc5ccc(-c6ccc7oc8ccccc8c7c6)cc5c34)n2)cc1 ; c1ccc(-c2nc(-c3ccc4ccccc4c3)nc(-c3cc(-c4ccc5oc6ccccc6c5c4)cc4oc5ccccc5c34)n2)cc1]

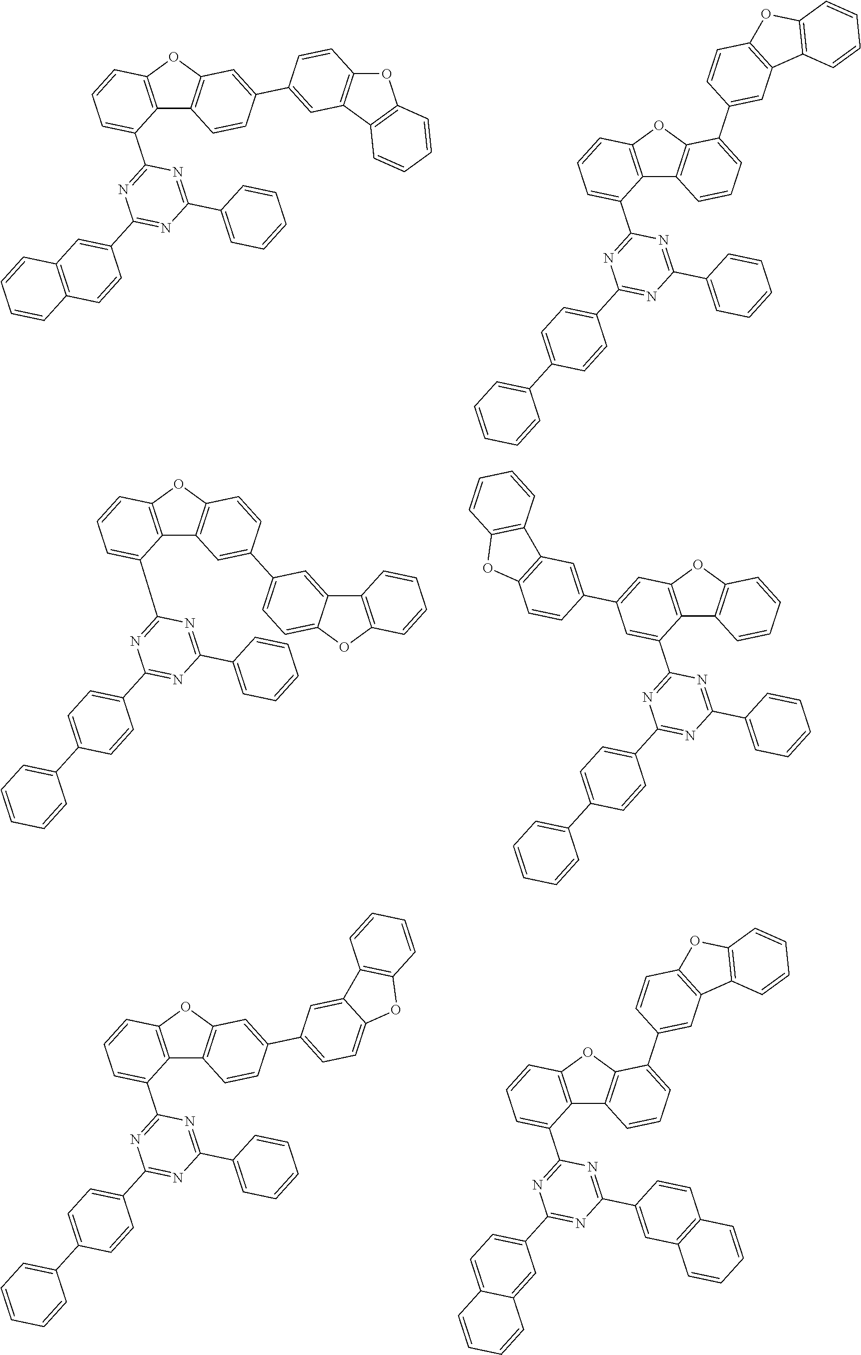

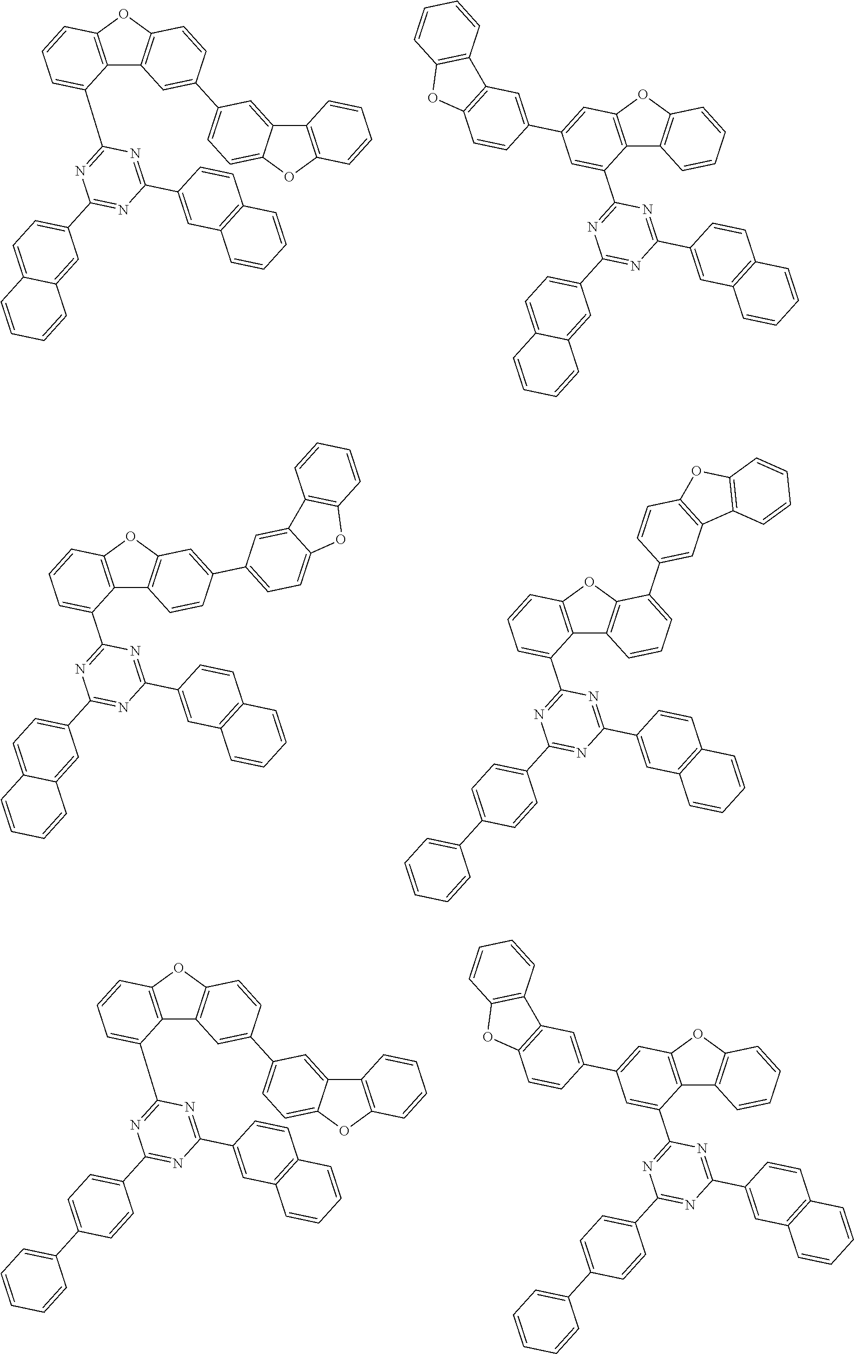

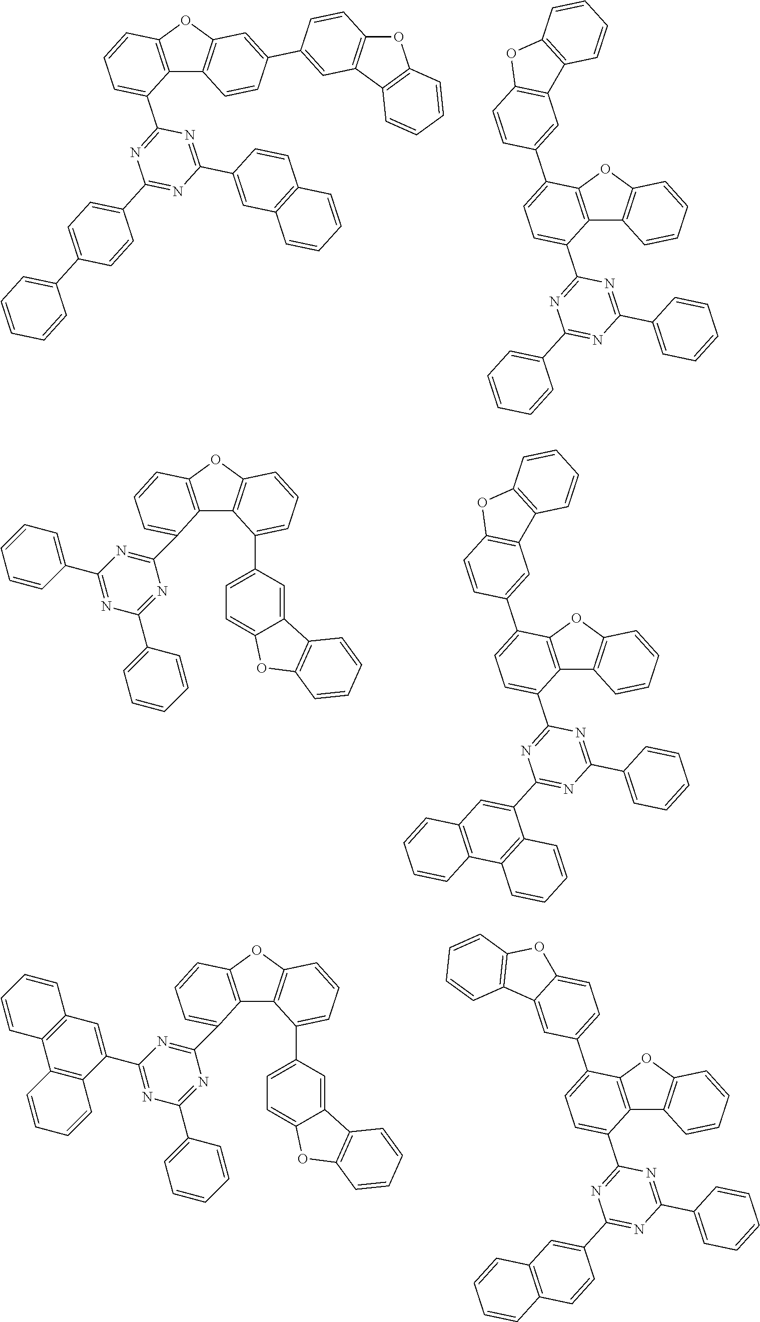

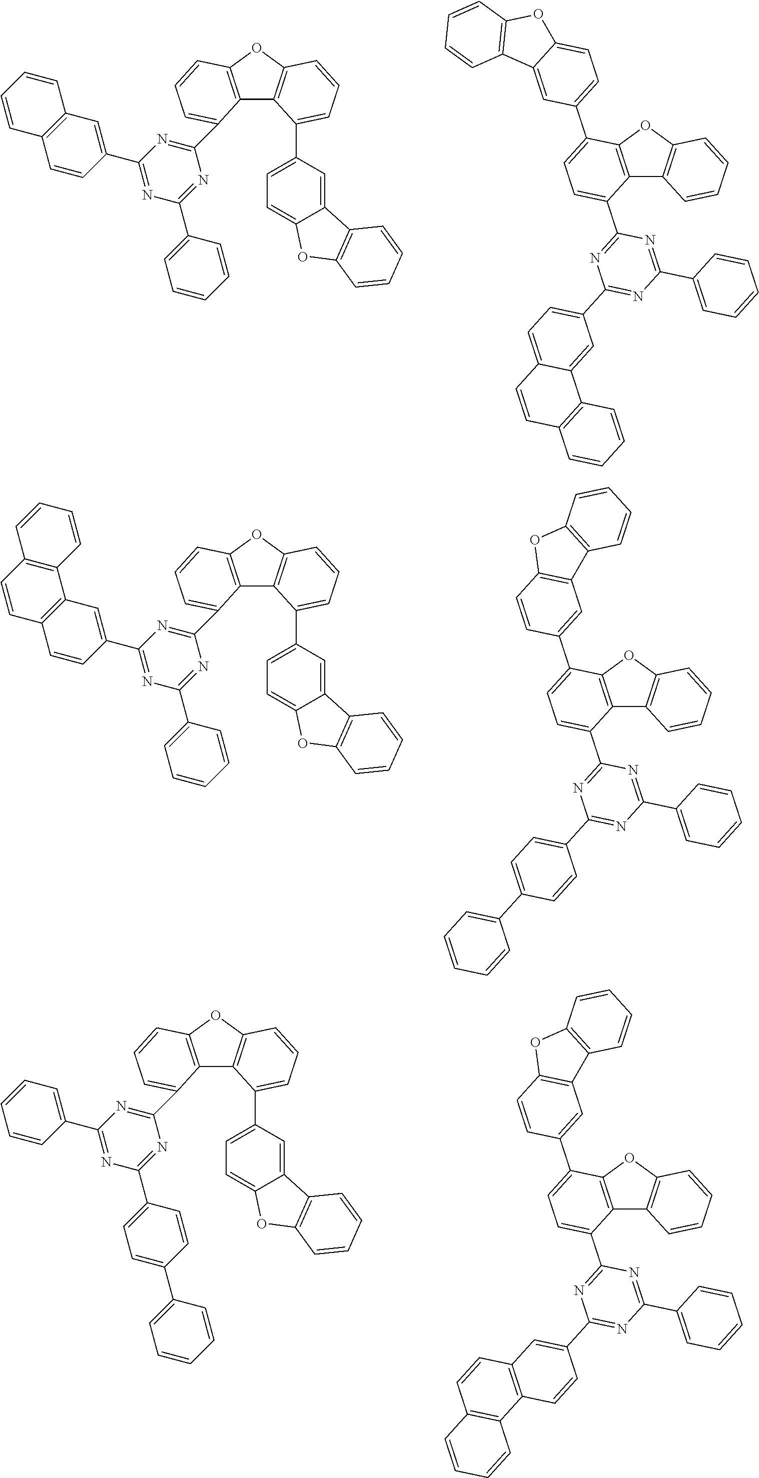

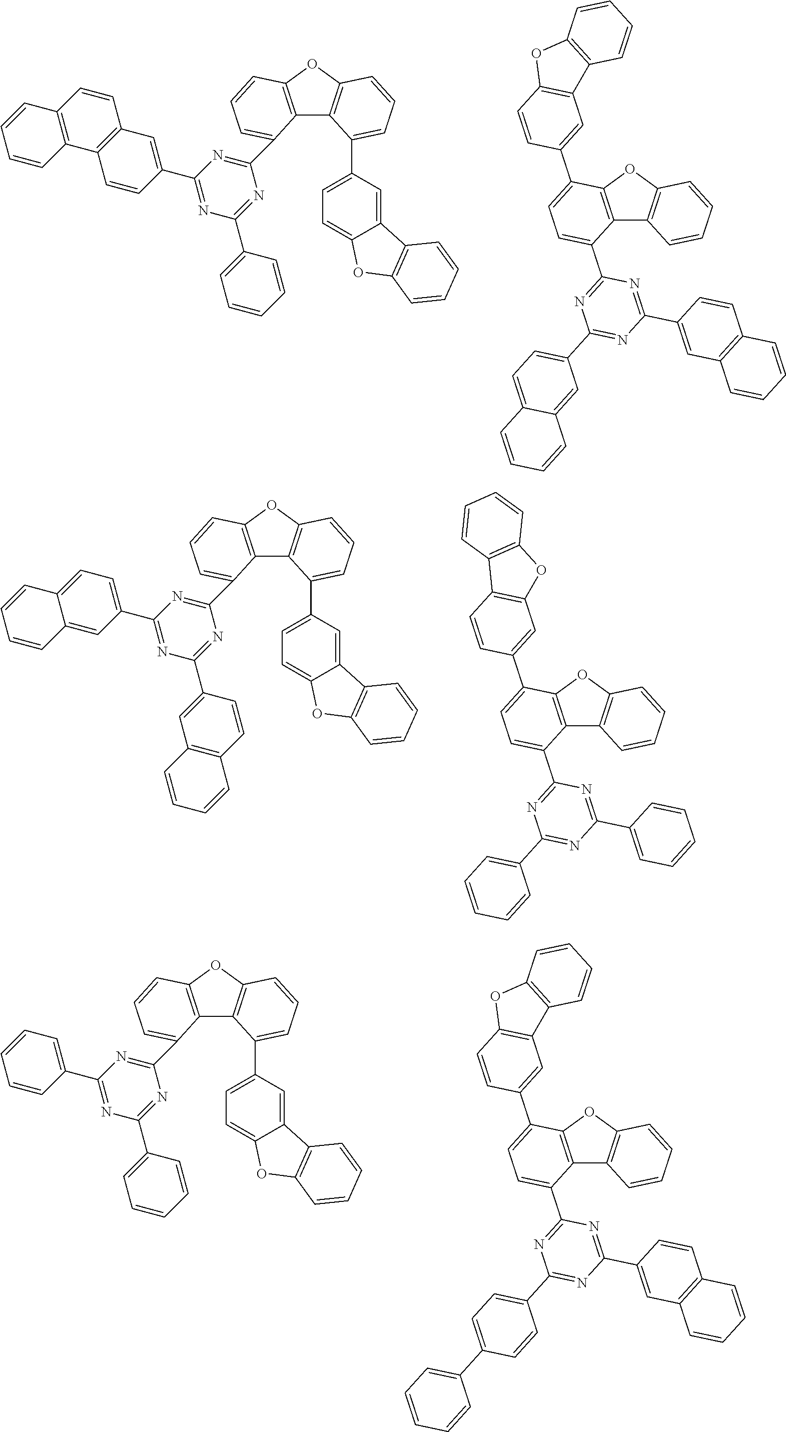

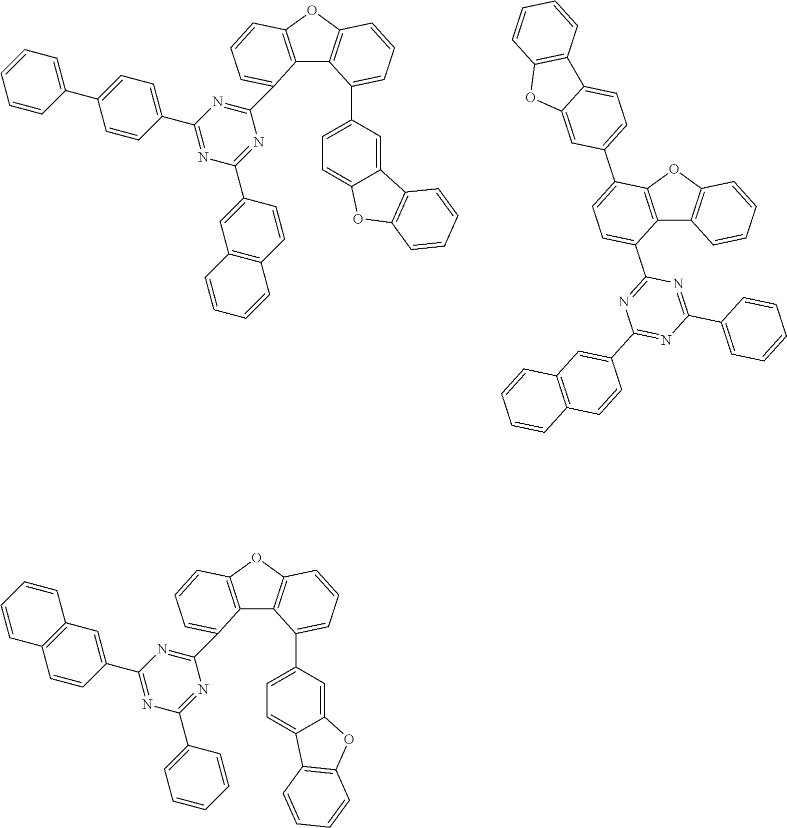 [SMILES: c1ccc(-c2ccc(-c3nc(-c4ccc5ccccc5c4)nc(-c4cccc5oc6cccc(-c7ccc8oc9ccccc9c8c7)c6c45)n3)cc2)cc1 ; c1ccc(-c2nc(-c3ccc4ccccc4c3)nc(-c3ccc(-c4ccc5c(c4)oc4ccccc45)c4oc5ccccc5c34)n2)cc1 ; c1ccc(-c2nc(-c3ccc4ccccc4c3)nc(-c3cccc4oc5cccc(-c6ccc7c(c6)oc6ccccc67)c5c34)n2)cc1]

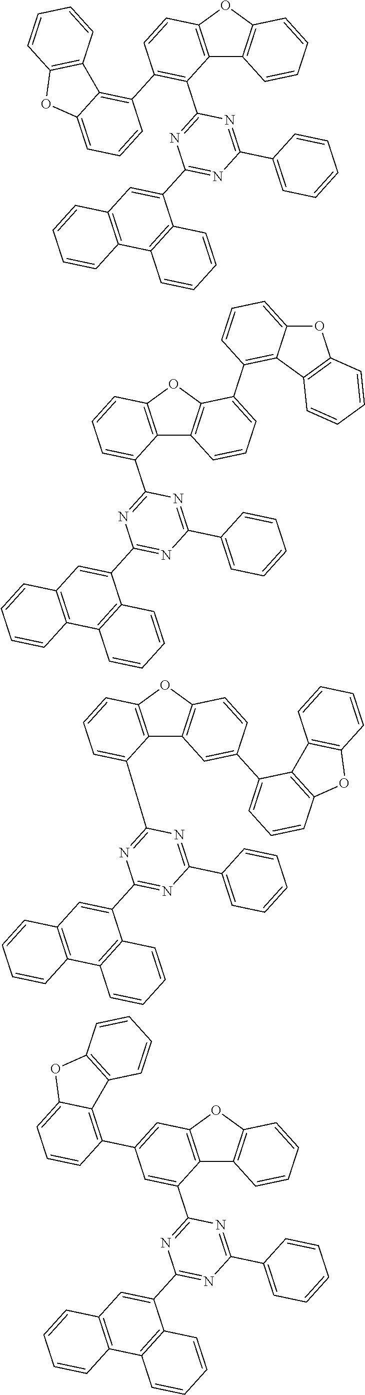

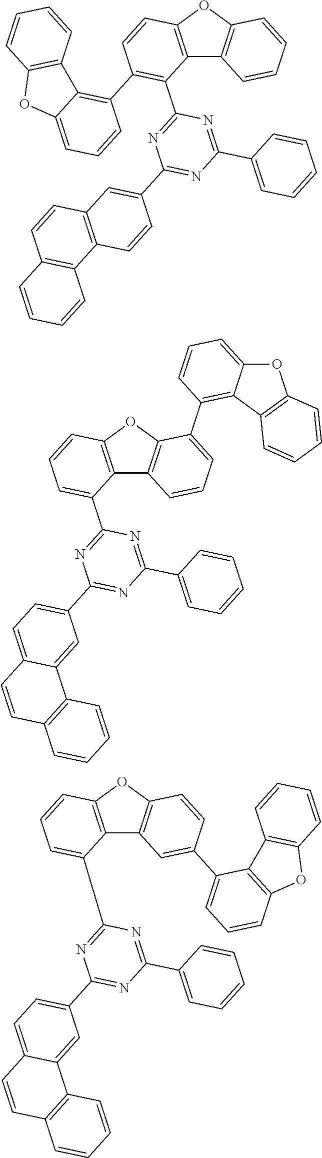

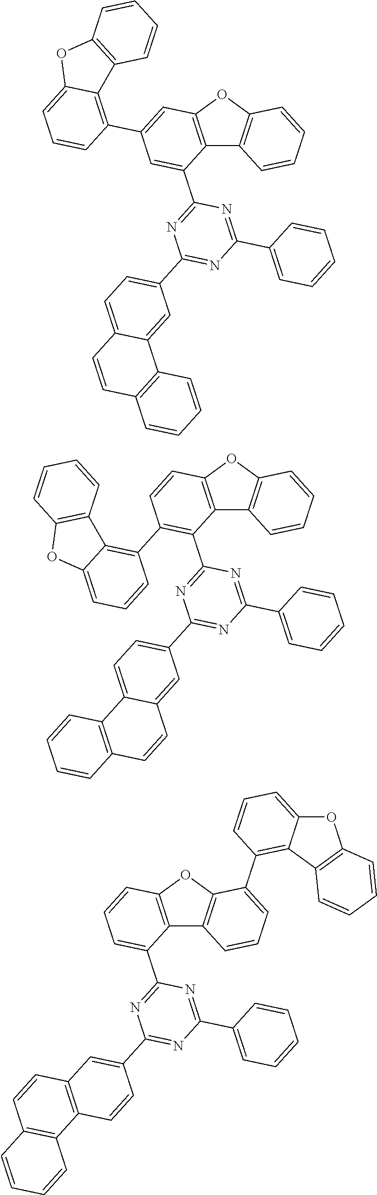

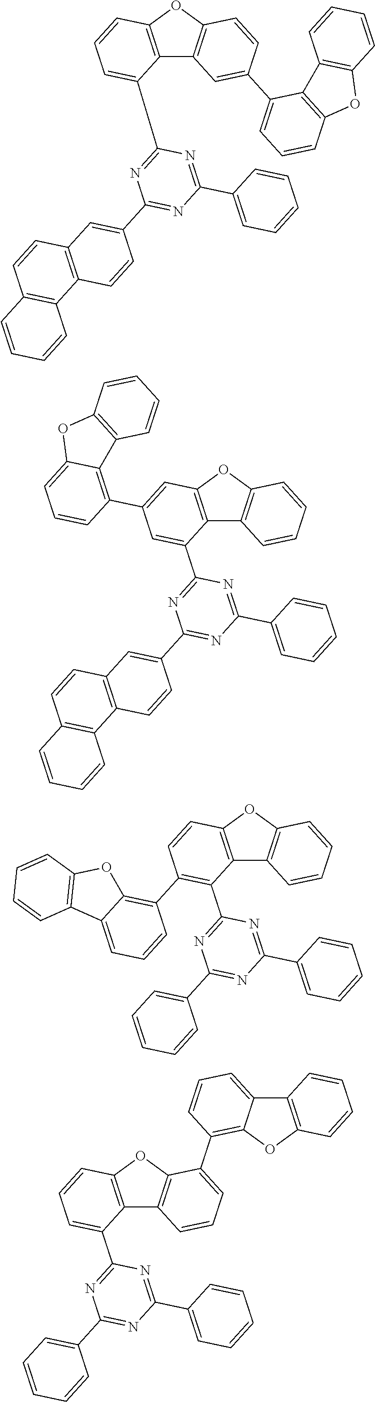

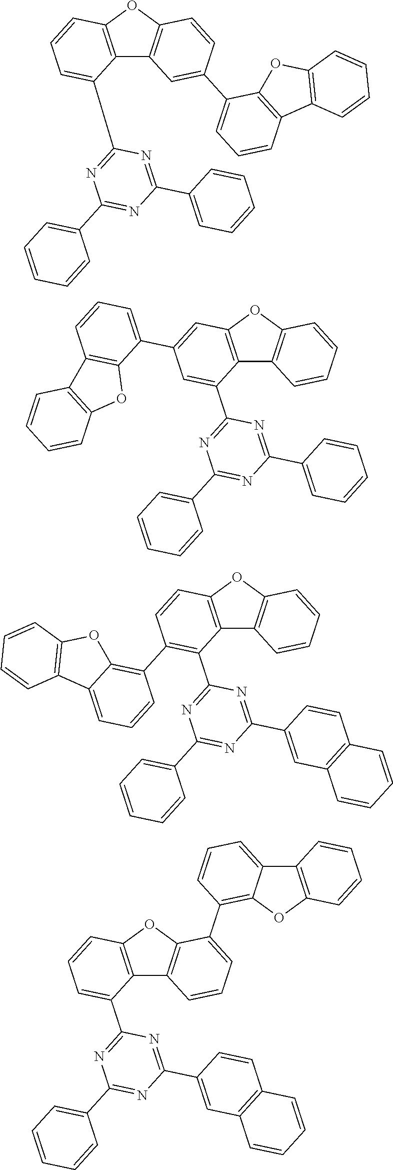

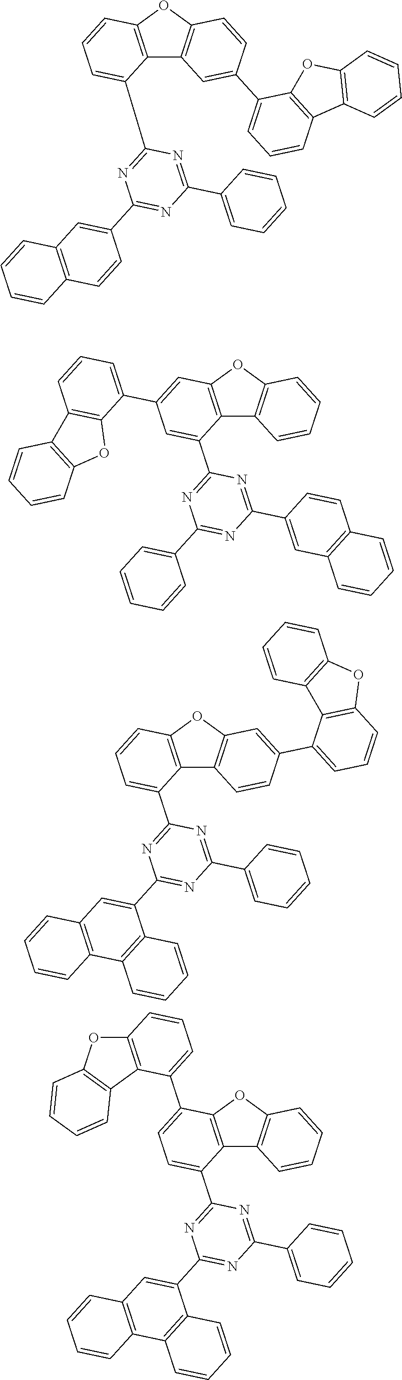

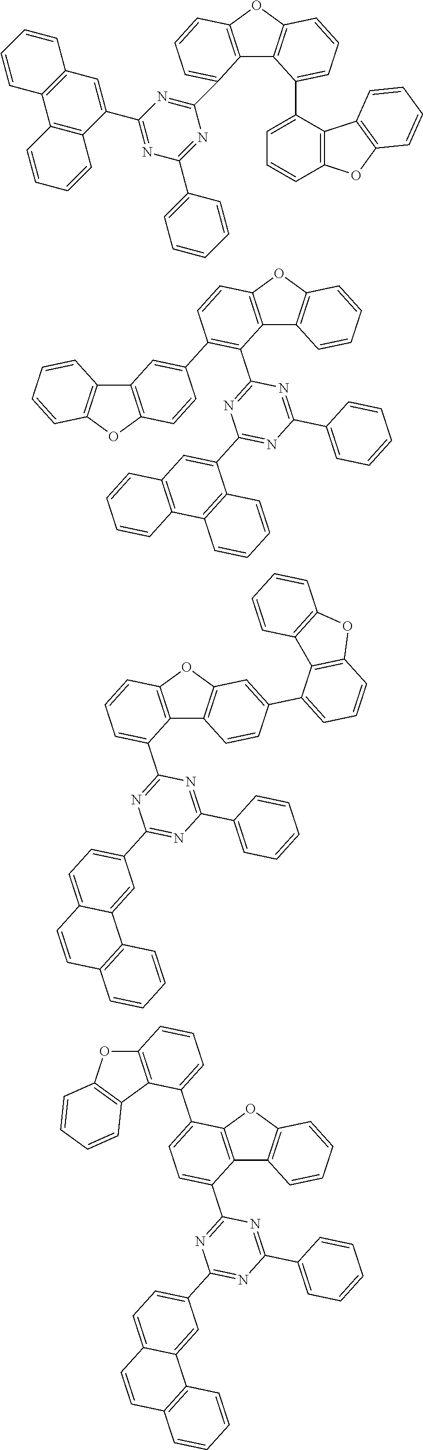

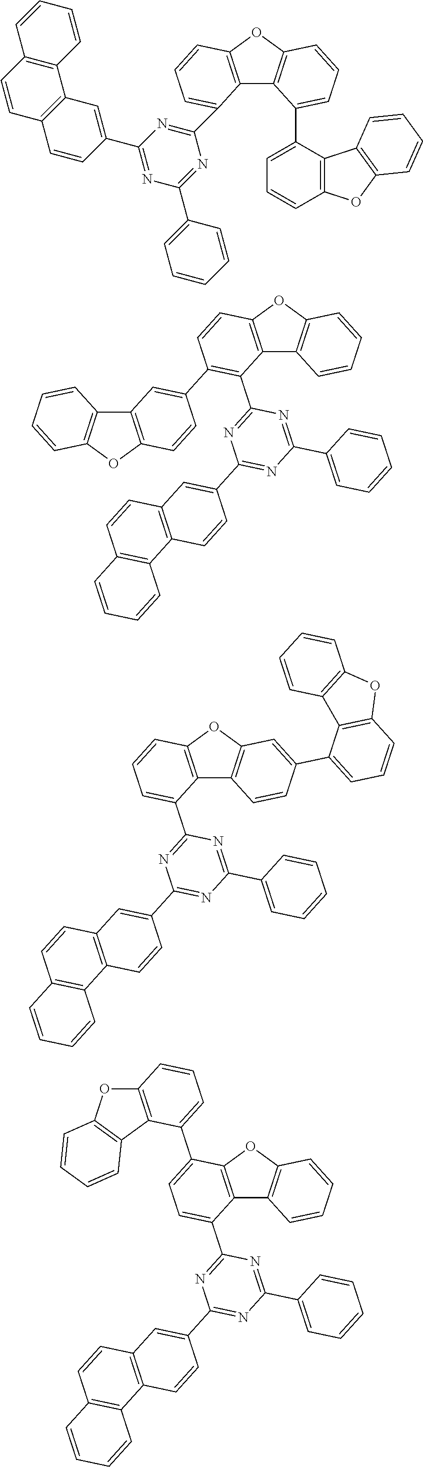

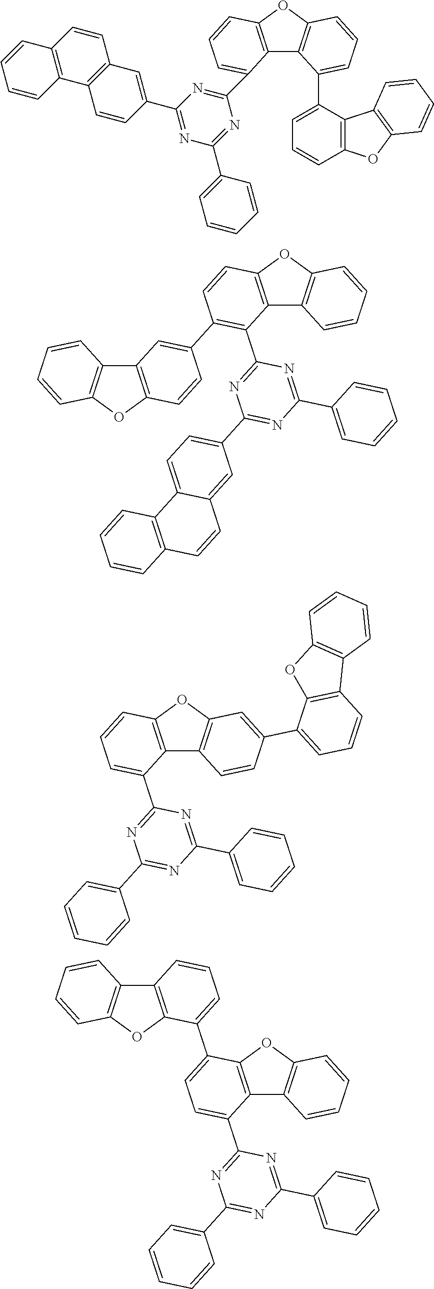

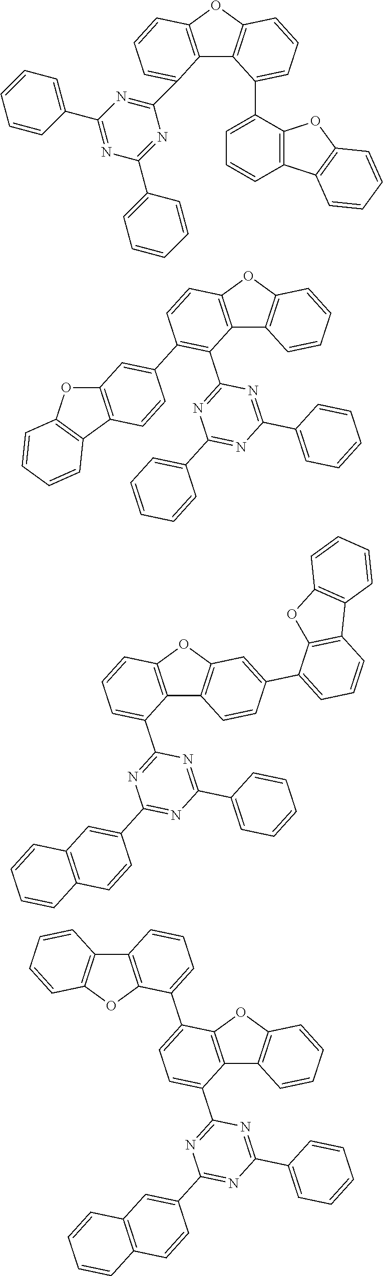

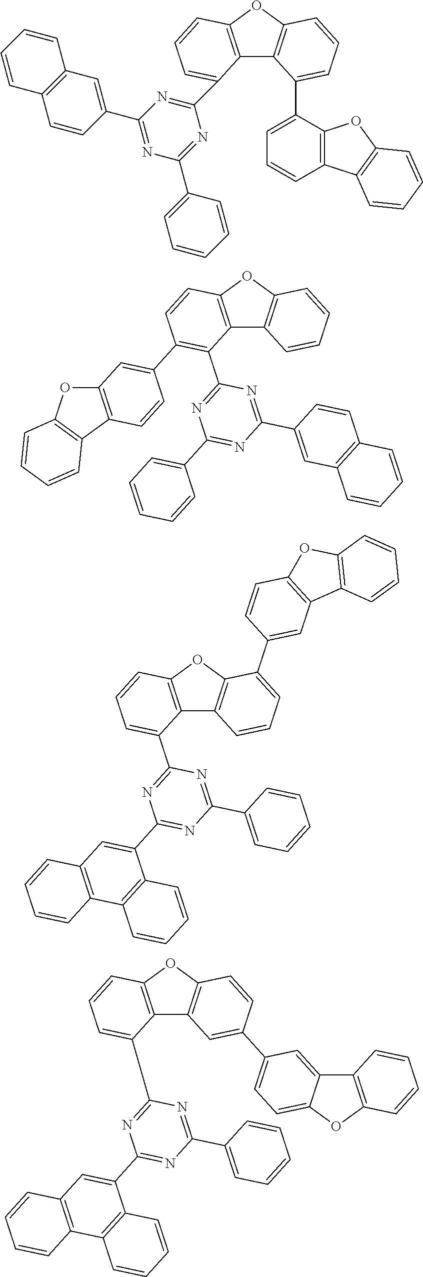

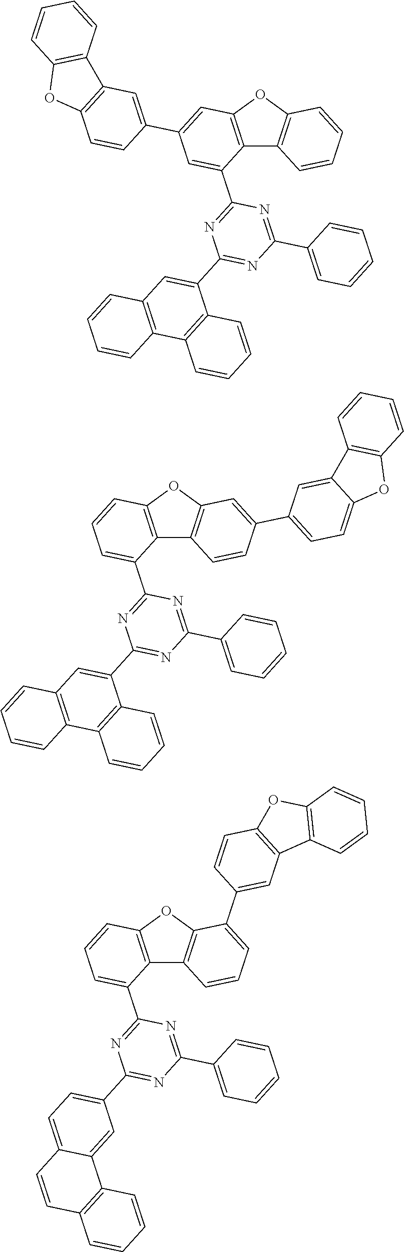 [SMILES: c1ccc(-c2nc(-c3cc4ccccc4c4ccccc34)nc(-c3cc(-c4ccc5oc6ccccc6c5c4)cc4oc5ccccc5c34)n2)cc1 ; c1ccc(-c2nc(-c3cc4ccccc4c4ccccc34)nc(-c3cccc4oc5cc(-c6ccc7oc8ccccc8c7c6)ccc5c34)n2)cc1 ; c1ccc(-c2nc(-c3ccc4ccc5ccccc5c4c3)nc(-c3cccc4oc5c(-c6ccc7oc8ccccc8c7c6)cccc5c34)n2)cc1]

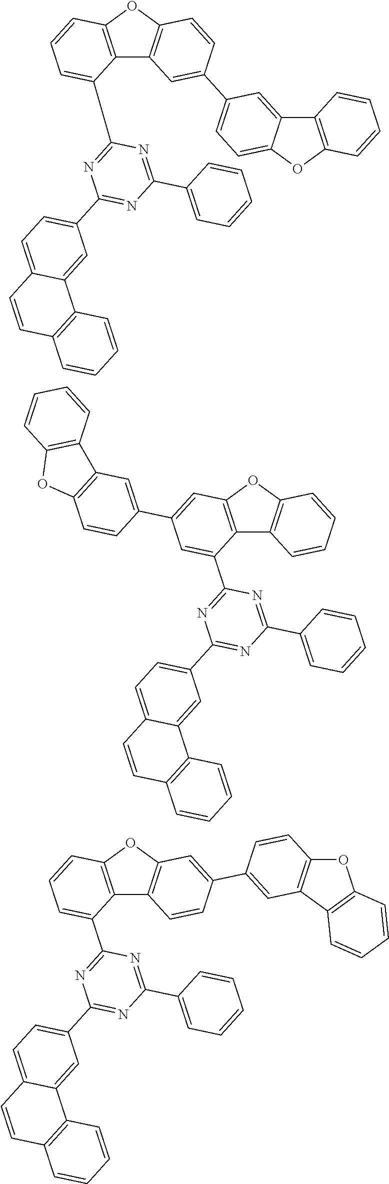

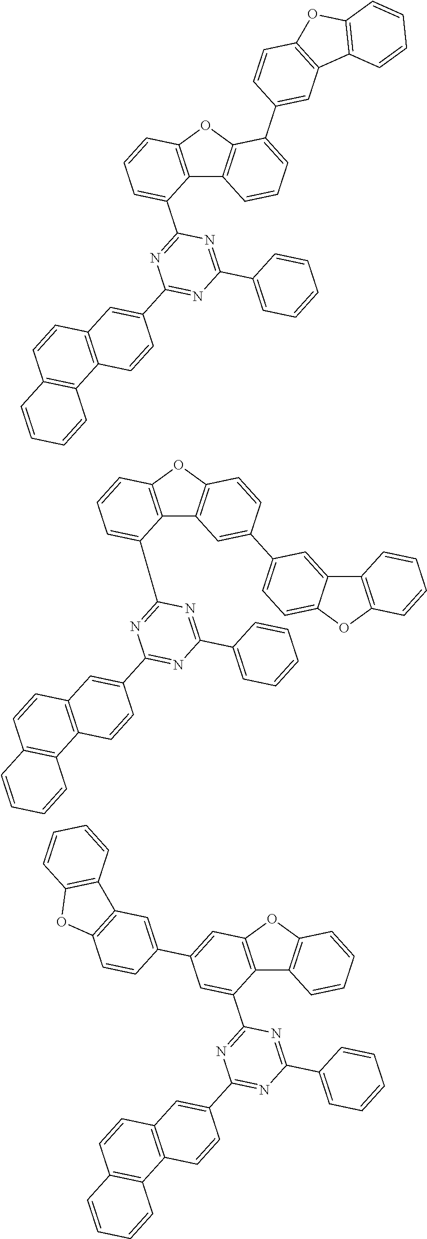

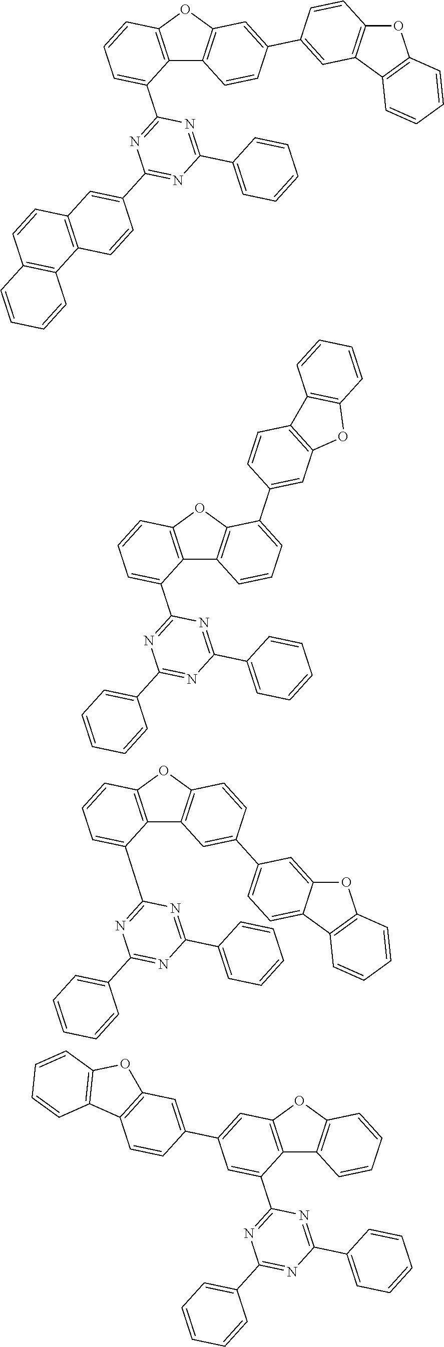

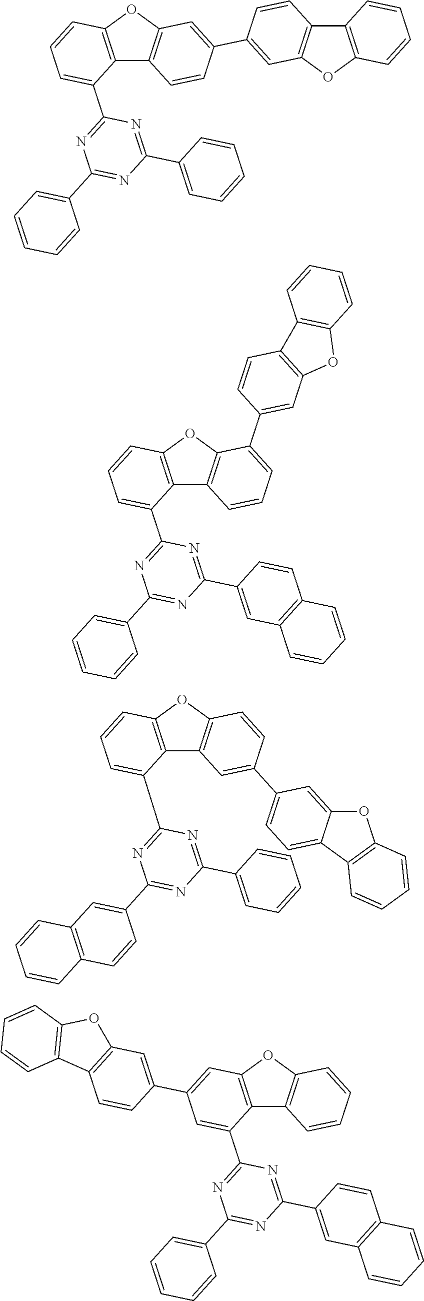

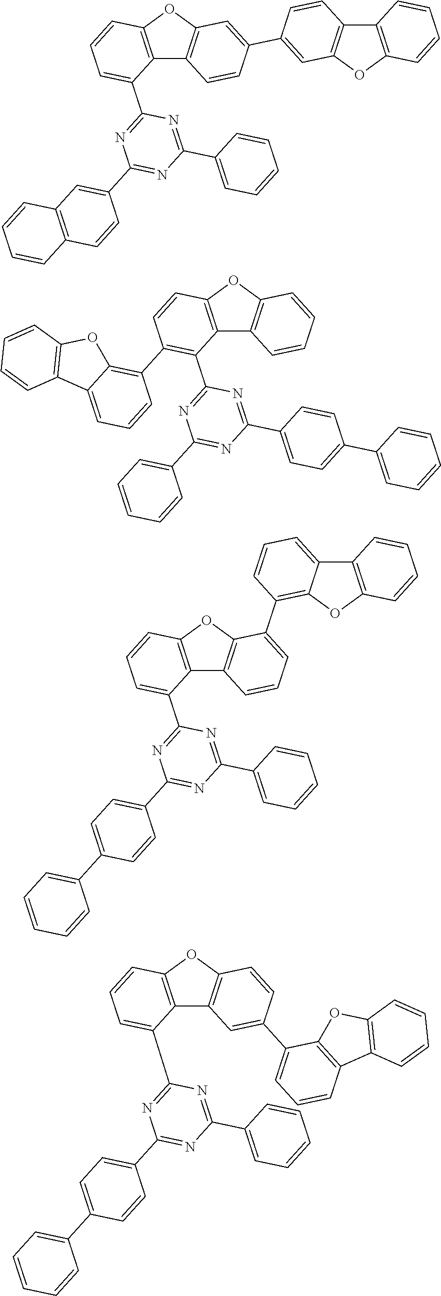 [SMILES: c1ccc(-c2nc(-c3ccc4ccccc4c3)nc(-c3cccc4oc5cc(-c6ccc7c(c6)oc6ccccc67)ccc5c34)n2)cc1 ; c1ccc(-c2ccc(-c3nc(-c4ccccc4)nc(-c4c(-c5cccc6c5oc5ccccc56)ccc5oc6ccccc6c45)n3)cc2)cc1 ; c1ccc(-c2ccc(-c3nc(-c4ccccc4)nc(-c4cccc5oc6c(-c7cccc8c7oc7ccccc78)cccc6c45)n3)cc2)cc1 ; c1ccc(-c2ccc(-c3nc(-c4ccccc4)nc(-c4cccc5oc6ccc(-c7cccc8c7oc7ccccc78)cc6c45)n3)cc2)cc1]

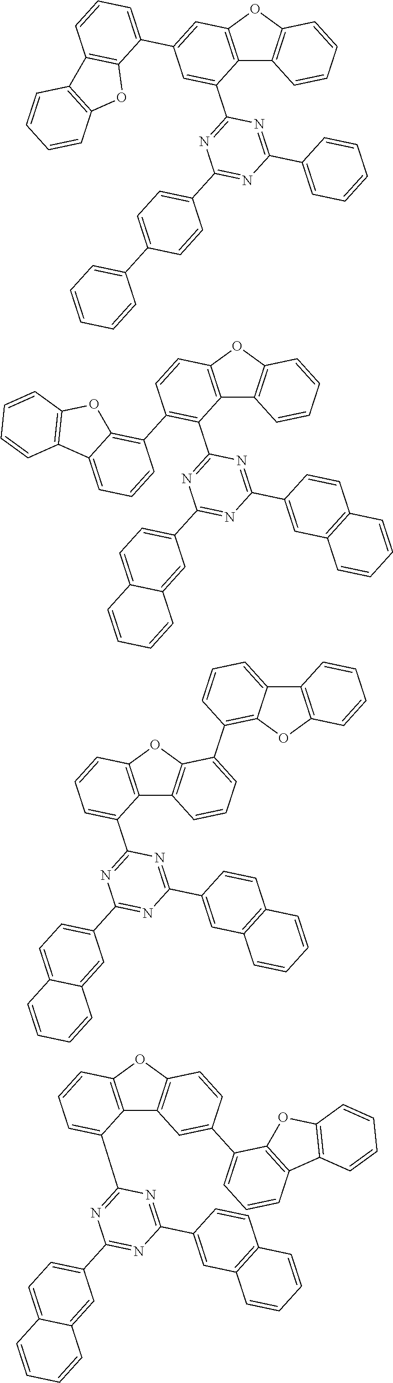

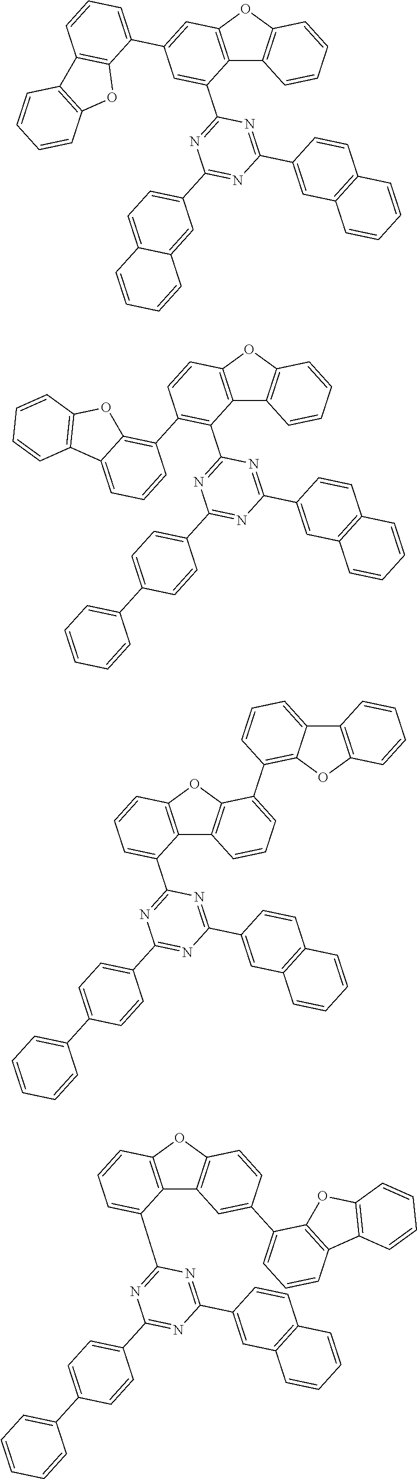 [SMILES: c1ccc2cc(-c3nc(-c4ccc5ccccc5c4)nc(-c4cc(-c5cccc6c5oc5ccccc56)cc5oc6ccccc6c45)n3)ccc2c1 ; c1ccc(-c2ccc(-c3nc(-c4ccc5ccccc5c4)nc(-c4c(-c5cccc6c5oc5ccccc56)ccc5oc6ccccc6c45)n3)cc2)cc1 ; c1ccc(-c2ccc(-c3nc(-c4ccc5ccccc5c4)nc(-c4cccc5oc6c(-c7cccc8c7oc7ccccc78)cccc6c45)n3)cc2)cc1 ; c1ccc(-c2ccc(-c3nc(-c4ccc5ccccc5c4)nc(-c4cccc5oc6ccc(-c7cccc8c7oc7ccccc78)cc6c45)n3)cc2)cc1]

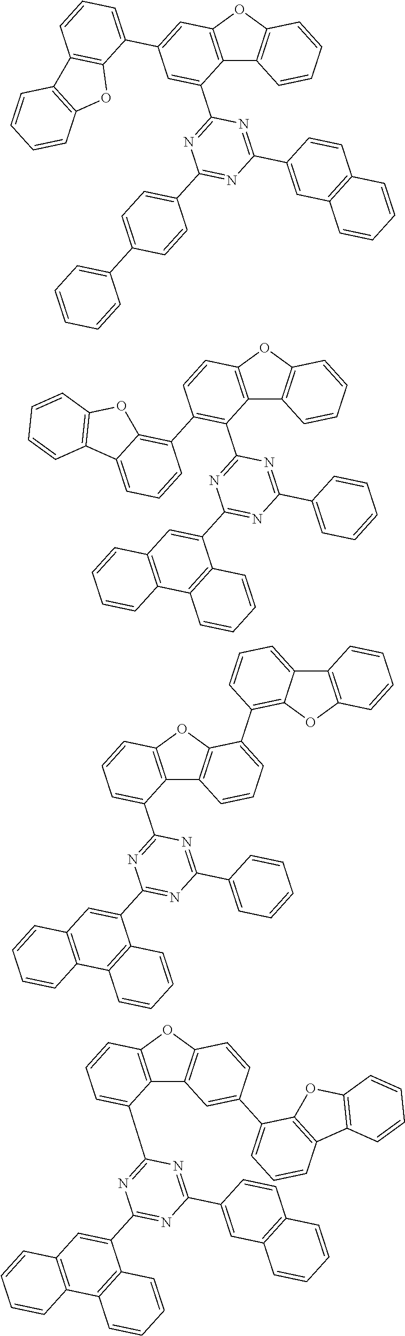

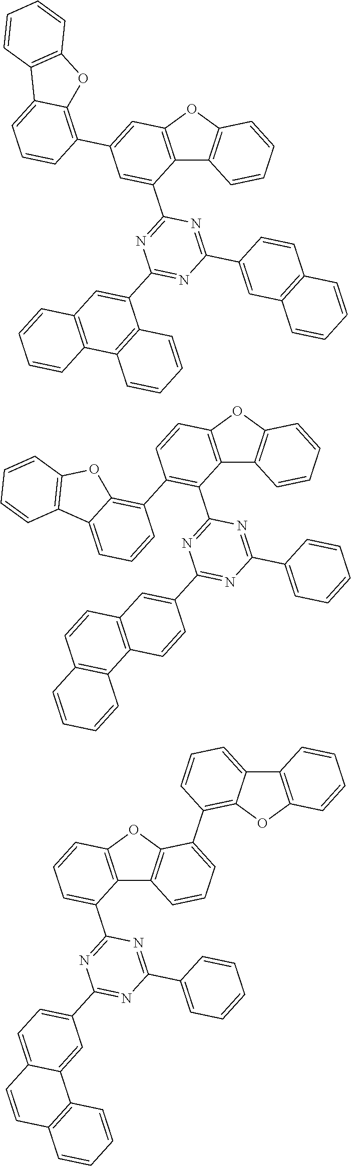

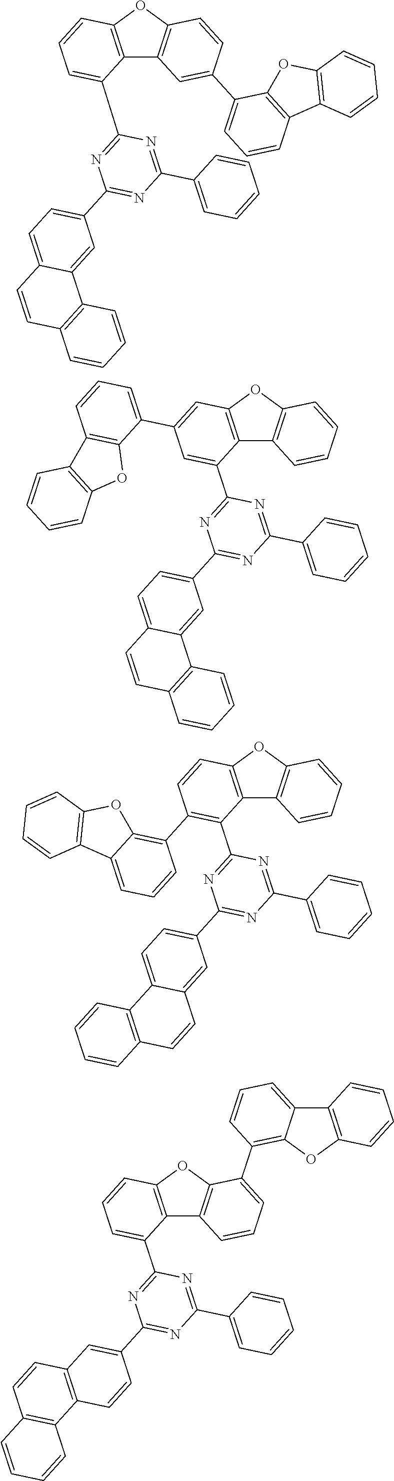

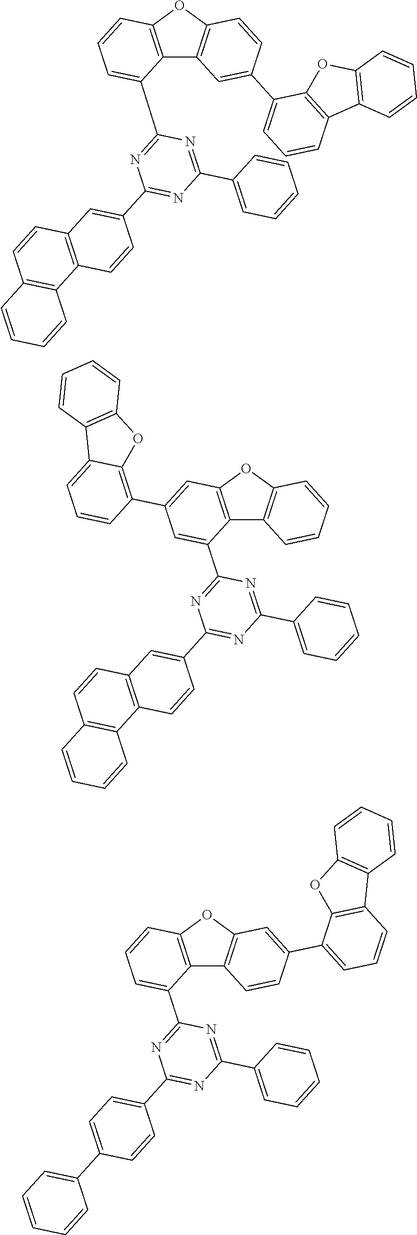

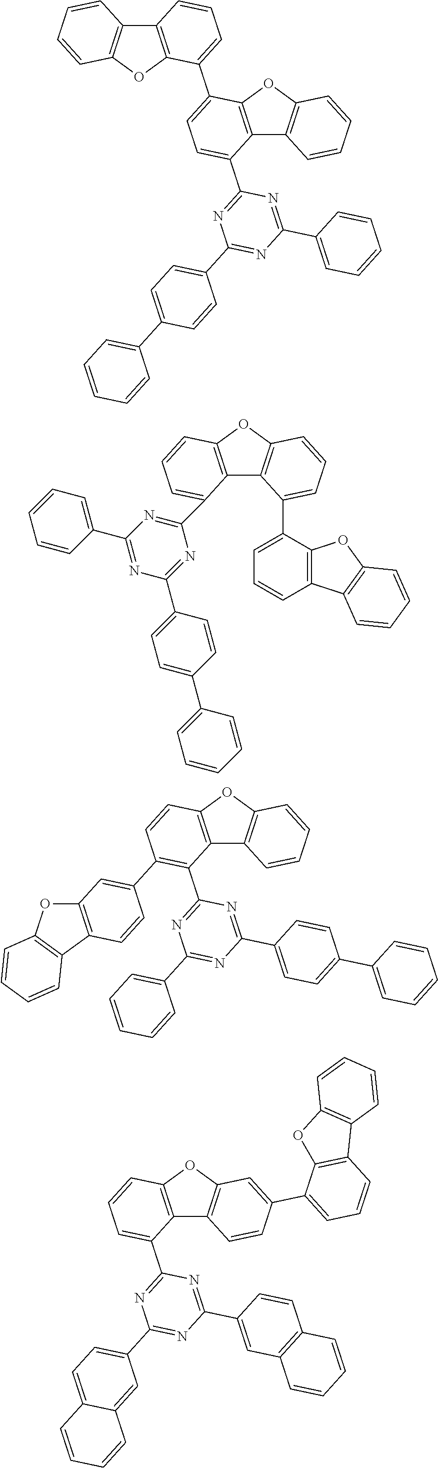

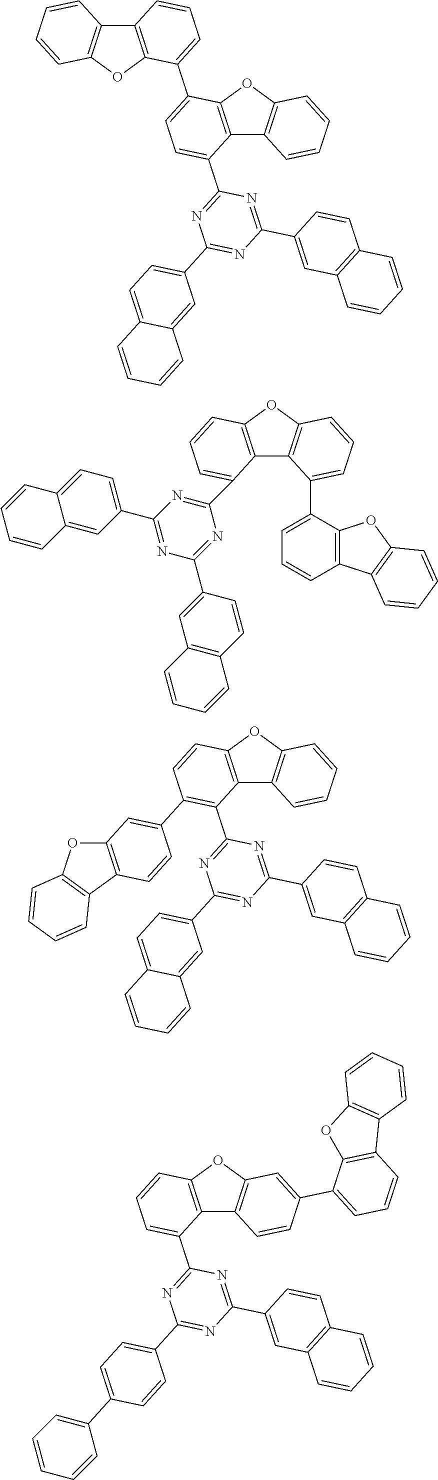

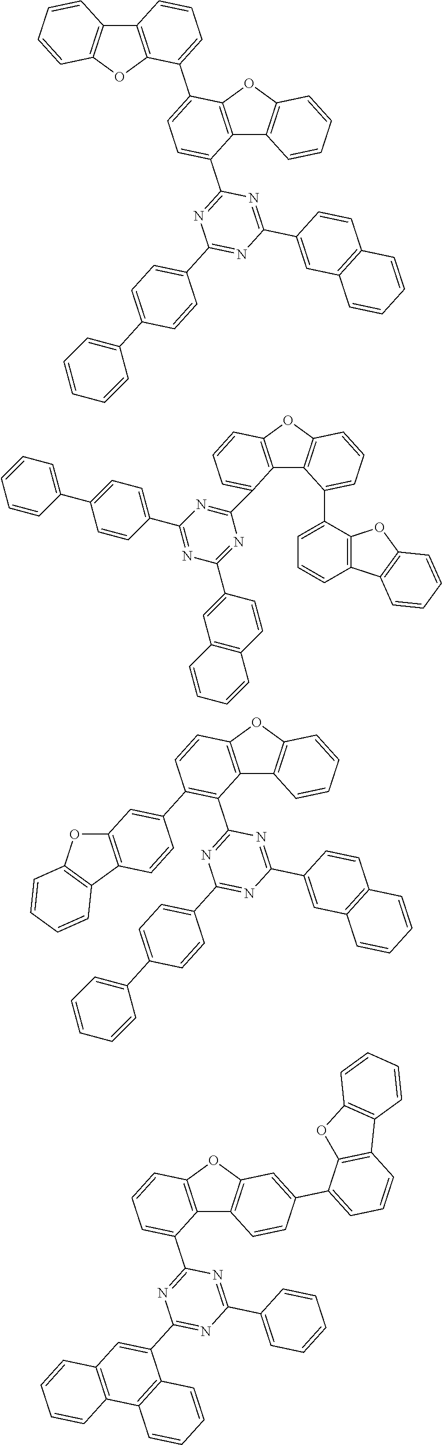

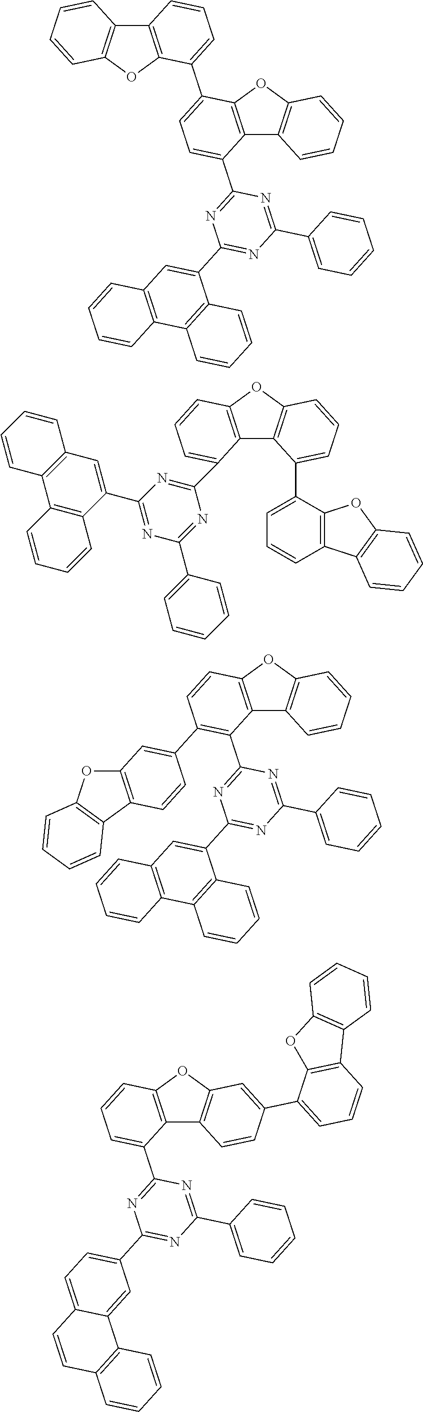

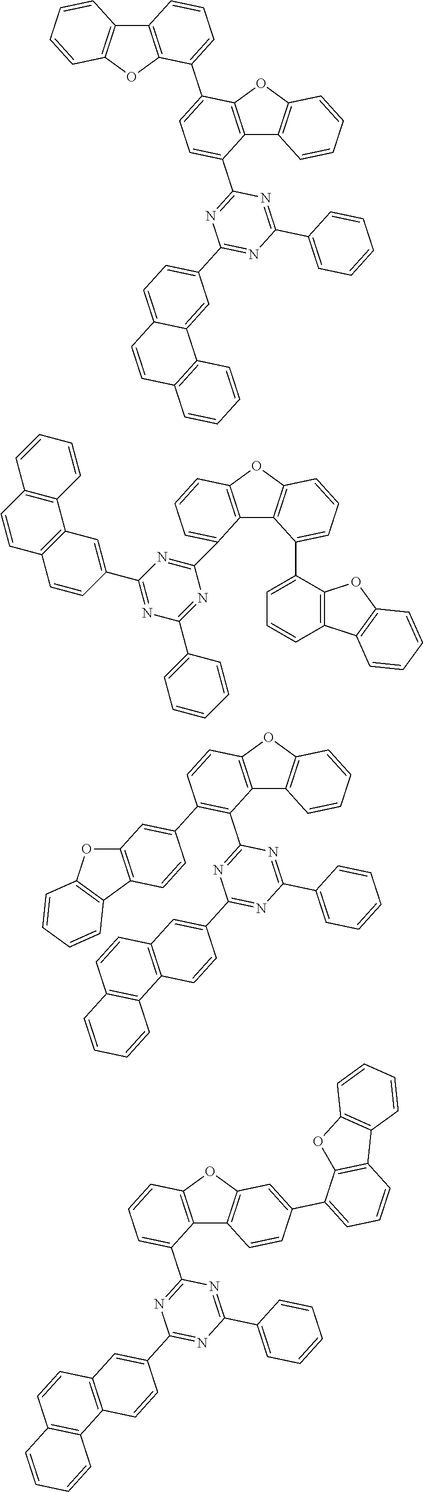

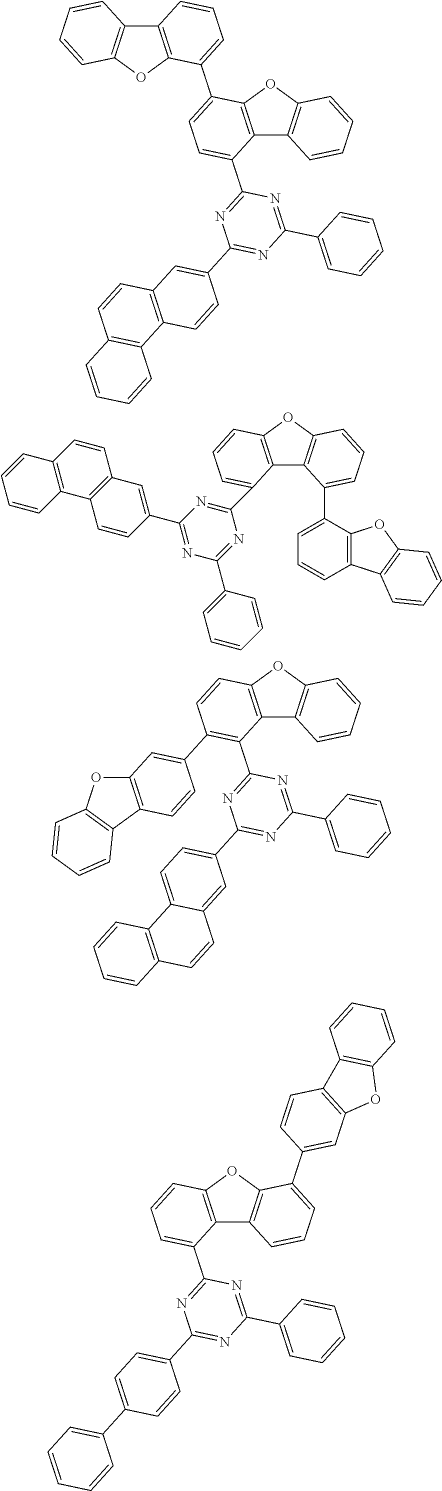

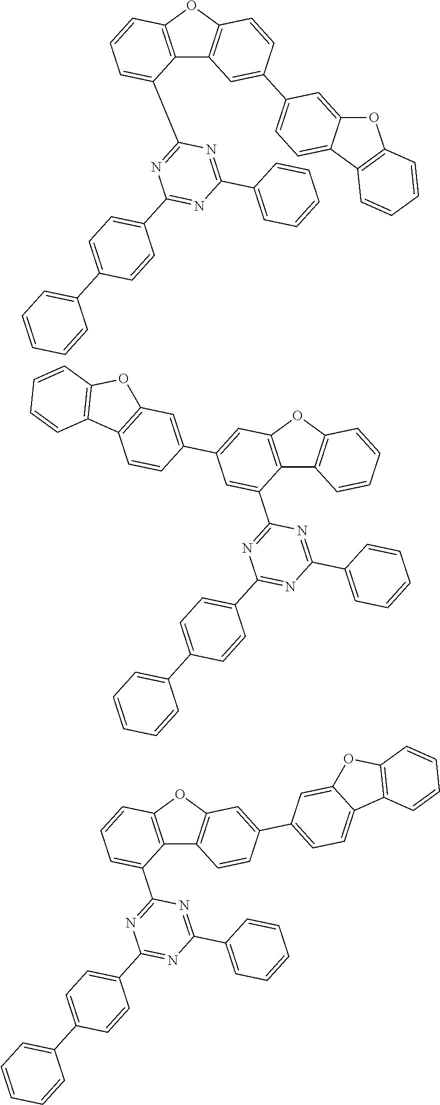

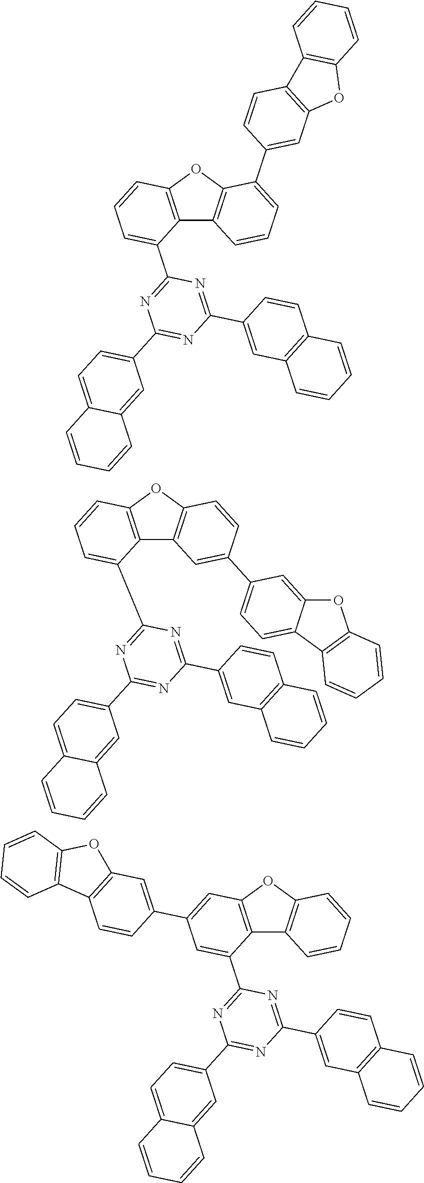

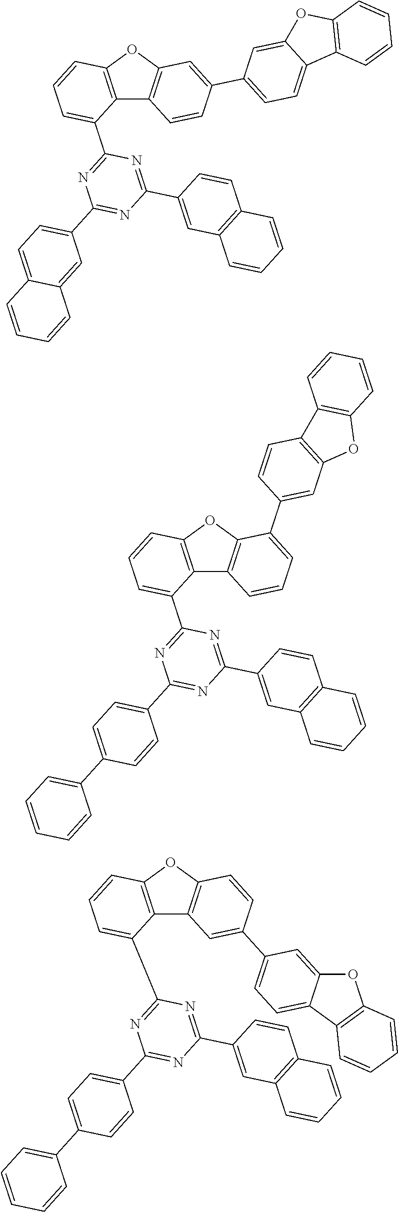 [SMILES: c1ccc2cc(-c3nc(-c4ccc5ccccc5c4)nc(-c4cccc5oc6cc(-c7ccc8c(c7)oc7ccccc78)ccc6c45)n3)ccc2c1 ; c1ccc(-c2ccc(-c3nc(-c4ccc5ccccc5c4)nc(-c4cccc5oc6c(-c7ccc8c(c7)oc7ccccc78)cccc6c45)n3)cc2)cc1 ; c1ccc(-c2ccc(-c3nc(-c4ccc5ccccc5c4)nc(-c4cccc5oc6ccc(-c7ccc8c(c7)oc7ccccc78)cc6c45)n3)cc2)cc1]

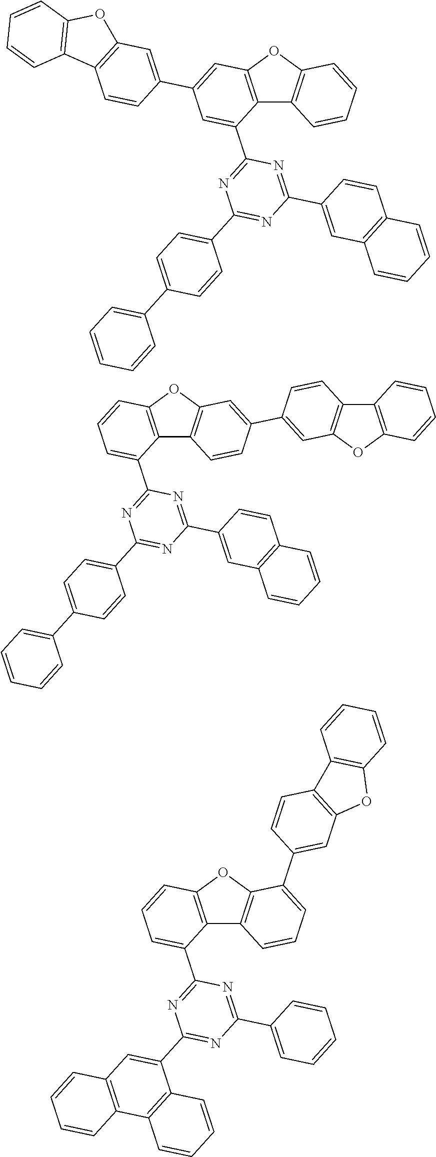

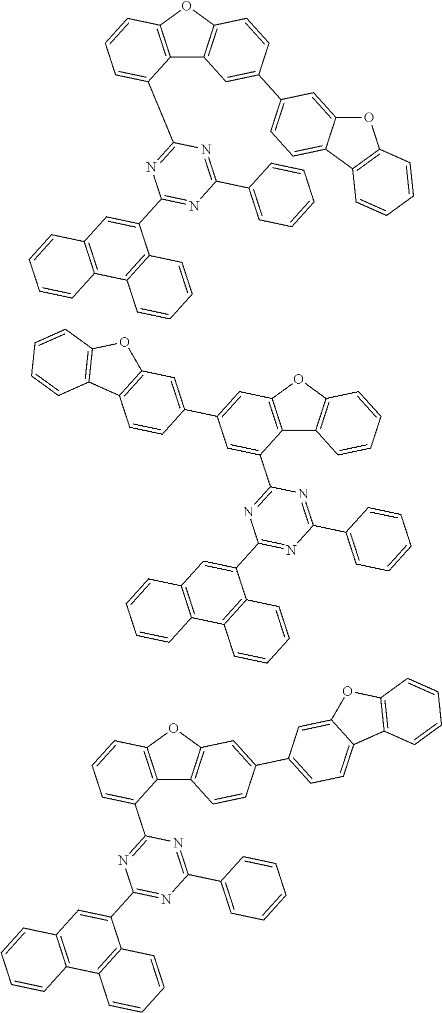

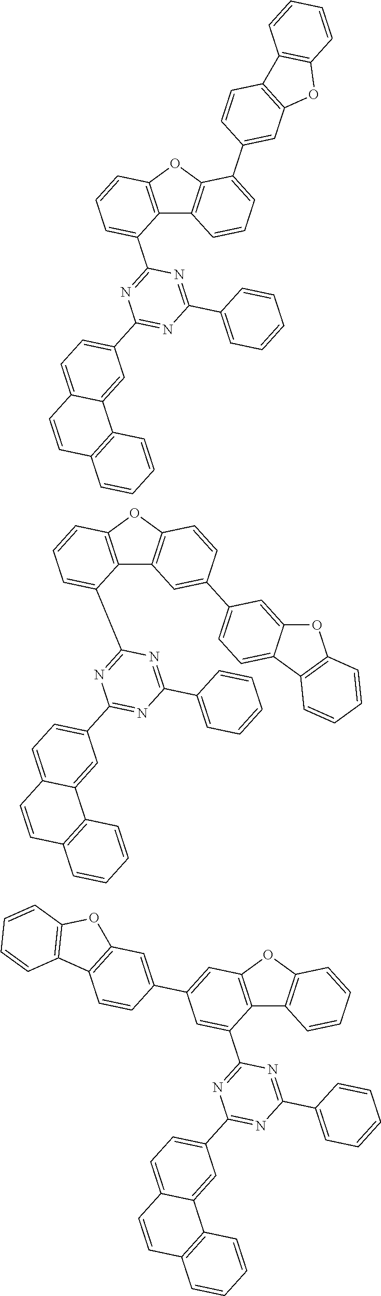

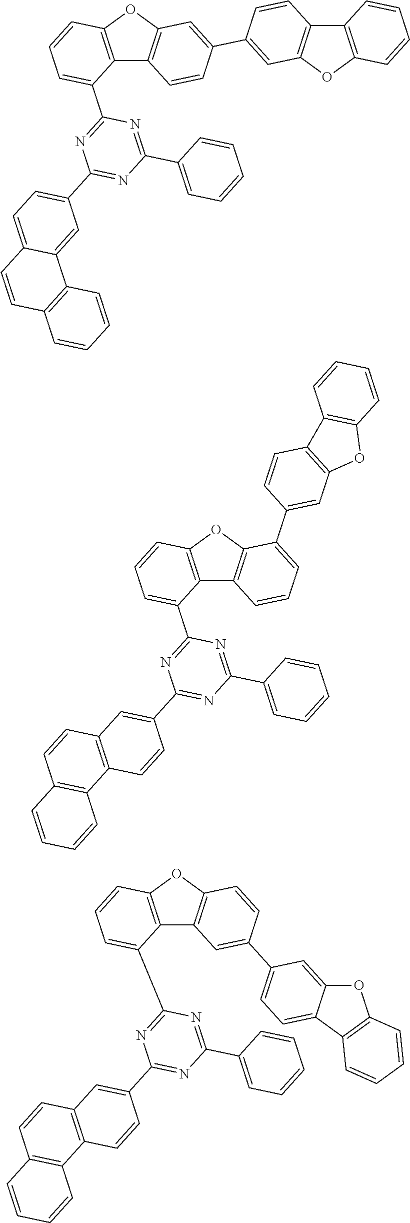 [SMILES: c1ccc(-c2nc(-c3ccc4ccc5ccccc5c4c3)nc(-c3cccc4oc5cc(-c6ccc7c(c6)oc6ccccc67)ccc5c34)n2)cc1 ; c1ccc(-c2nc(-c3ccc4c(ccc5ccccc54)c3)nc(-c3cccc4oc5c(-c6ccc7c(c6)oc6ccccc67)cccc5c34)n2)cc1 ; c1ccc(-c2nc(-c3ccc4c(ccc5ccccc54)c3)nc(-c3cccc4oc5ccc(-c6ccc7c(c6)oc6ccccc67)cc5c34)n2)cc1]

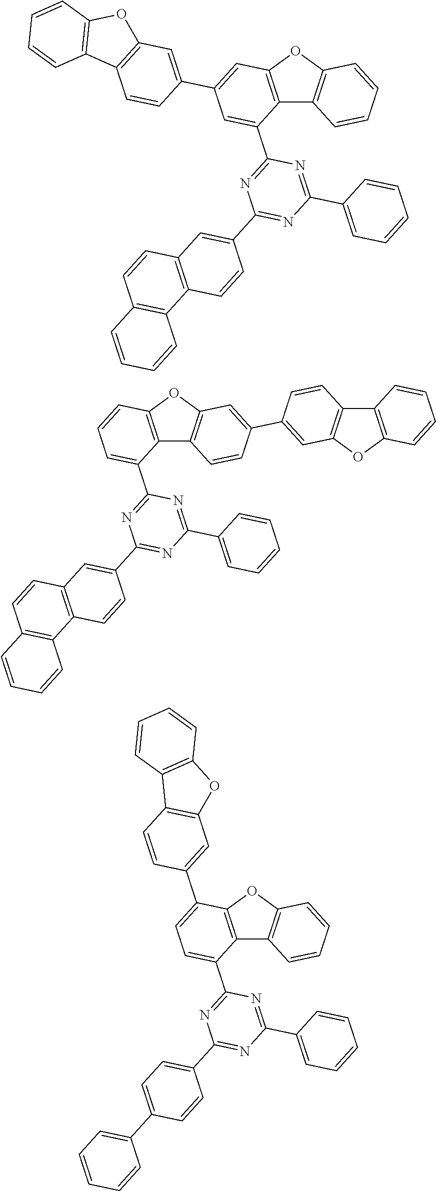

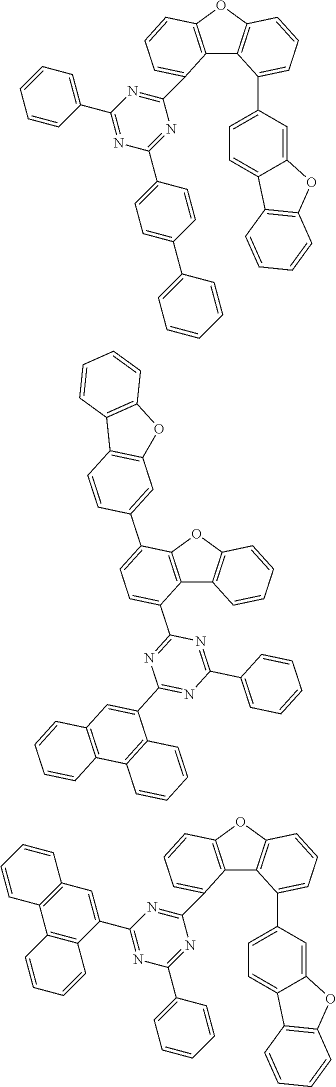

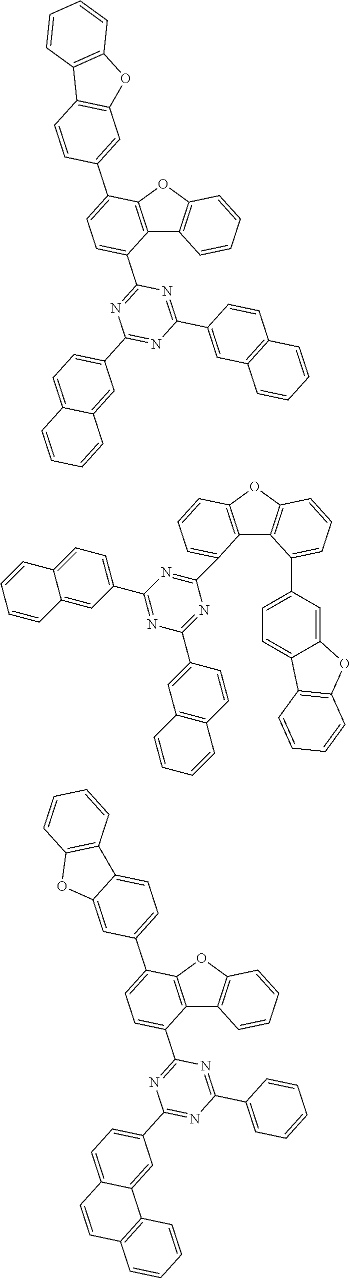 [SMILES: c1ccc2cc(-c3nc(-c4ccc5ccccc5c4)nc(-c4ccc(-c5ccc6c(c5)oc5ccccc56)c5oc6ccccc6c45)n3)ccc2c1 ; c1ccc2cc(-c3nc(-c4ccc5ccccc5c4)nc(-c4cccc5oc6cccc(-c7ccc8c(c7)oc7ccccc78)c6c45)n3)ccc2c1 ; c1ccc(-c2nc(-c3ccc4ccc5ccccc5c4c3)nc(-c3ccc(-c4ccc5c(c4)oc4ccccc45)c4oc5ccccc5c34)n2)cc1]

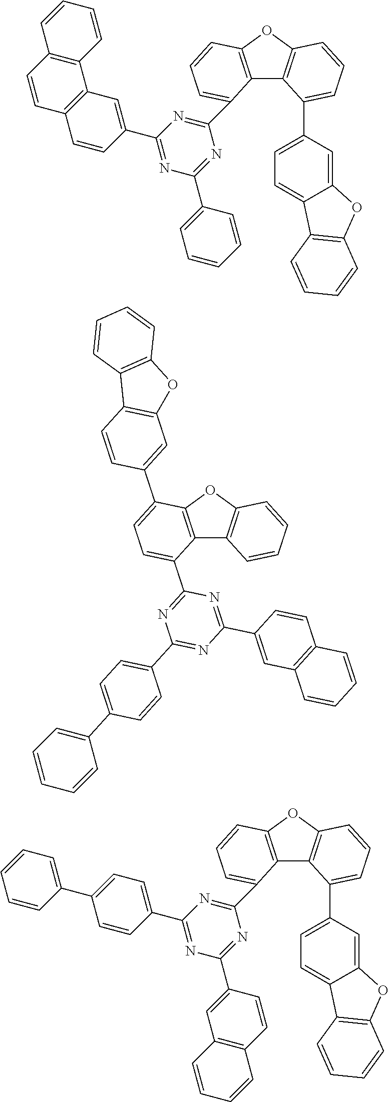 [SMILES: c1ccc(-c2nc(-c3ccc4ccc5ccccc5c4c3)nc(-c3cccc4oc5cccc(-c6ccc7c(c6)oc6ccccc67)c5c34)n2)cc1 ; c1ccc(-c2ccc(-c3nc(-c4ccc5ccccc5c4)nc(-c4ccc(-c5ccc6c(c5)oc5ccccc56)c5oc6ccccc6c45)n3)cc2)cc1 ; c1ccc(-c2ccc(-c3nc(-c4ccc5ccccc5c4)nc(-c4cccc5oc6cccc(-c7ccc8c(c7)oc7ccccc78)c6c45)n3)cc2)cc1]

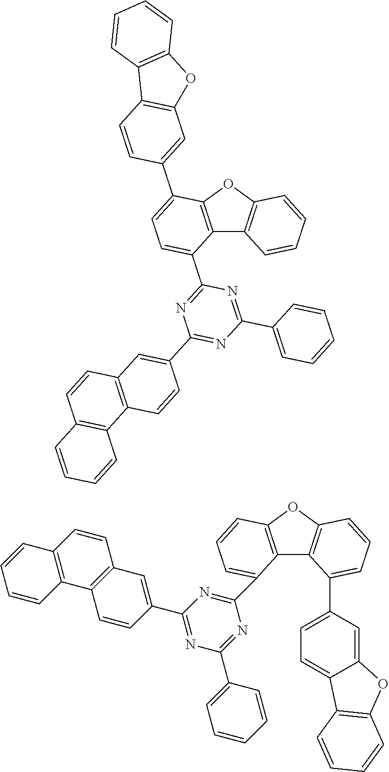 [SMILES: c1ccc(-c2nc(-c3ccc4c(ccc5ccccc54)c3)nc(-c3ccc(-c4ccc5c(c4)oc4ccccc45)c4oc5ccccc5c34)n2)cc1 ; c1ccc(-c2nc(-c3ccc4c(ccc5ccccc54)c3)nc(-c3cccc4oc5cccc(-c6ccc7c(c6)oc6ccccc67)c5c34)n2)cc1]

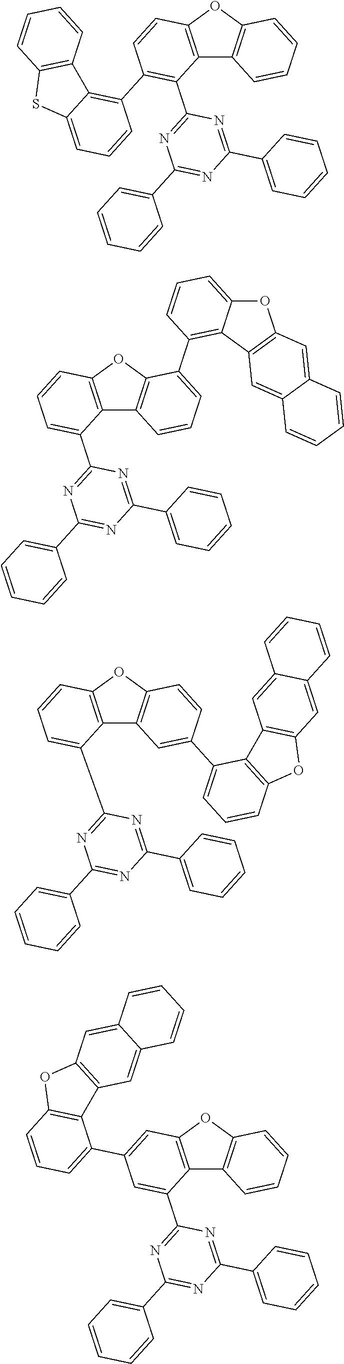 [SMILES: c1ccc(-c2nc(-c3ccccc3)nc(-c3c(-c4cccc5sc6ccccc6c45)ccc4oc5ccccc5c34)n2)cc1 ; c1ccc(-c2nc(-c3ccccc3)nc(-c3cccc4oc5c(-c6cccc7oc8cc9ccccc9cc8c67)cccc5c34)n2)cc1 ; c1ccc(-c2nc(-c3ccccc3)nc(-c3cccc4oc5ccc(-c6cccc7oc8cc9ccccc9cc8c67)cc5c34)n2)cc1 ; c1ccc(-c2nc(-c3ccccc3)nc(-c3cc(-c4cccc5oc6cc7ccccc7cc6c45)cc4oc5ccccc5c34)n2)cc1]

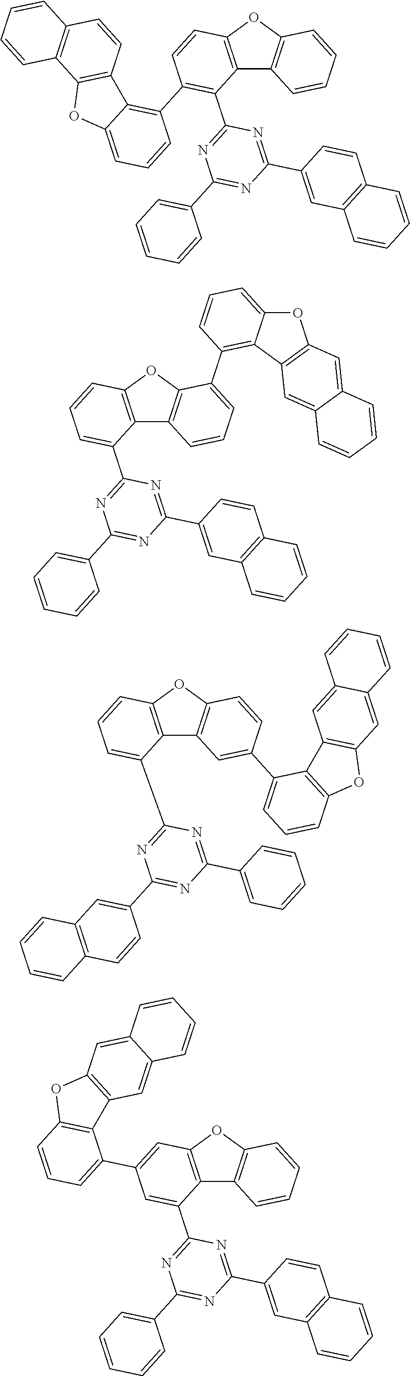

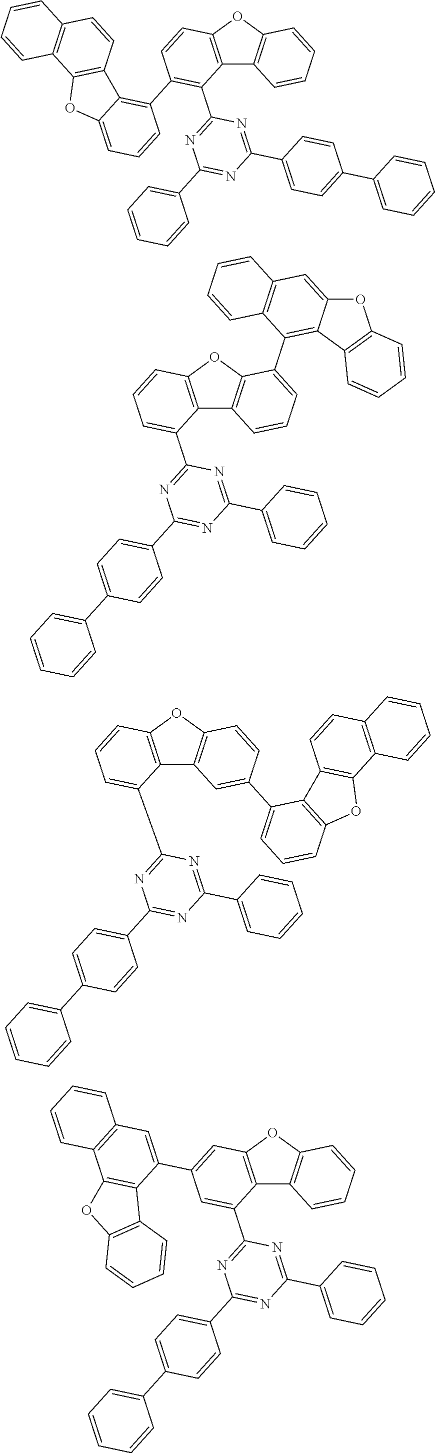

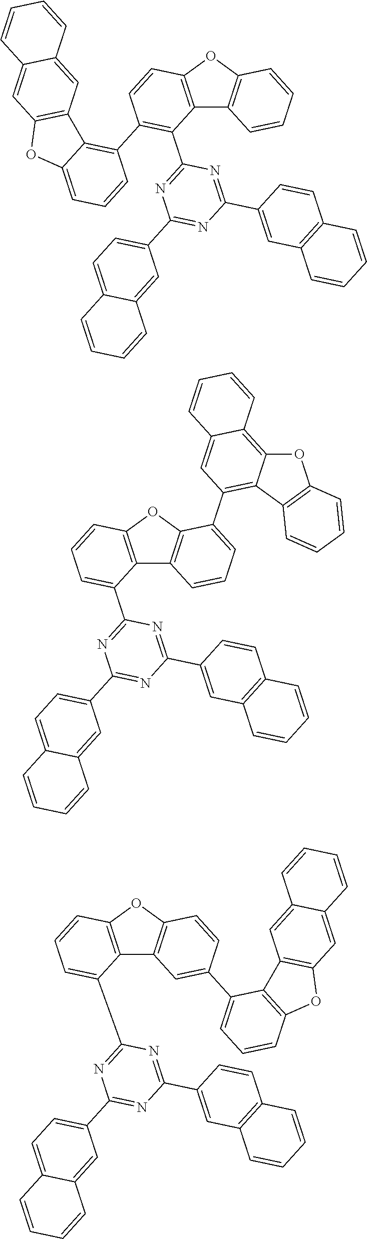 [SMILES: c1ccc2cc(-c3nc(-c4ccc5ccccc5c4)nc(-c4c(-c5cccc6oc7cc8ccccc8cc7c56)ccc5oc6ccccc6c45)n3)ccc2c1 ; c1ccc2cc(-c3nc(-c4ccc5ccccc5c4)nc(-c4cccc5oc6c(-c7cc8ccccc8c8oc9ccccc9c78)cccc6c45)n3)ccc2c1 ; c1ccc2cc(-c3nc(-c4ccc5ccccc5c4)nc(-c4cccc5oc6ccc(-c7cccc8oc9cc%10ccccc%10cc9c78)cc6c45)n3)ccc2c1]

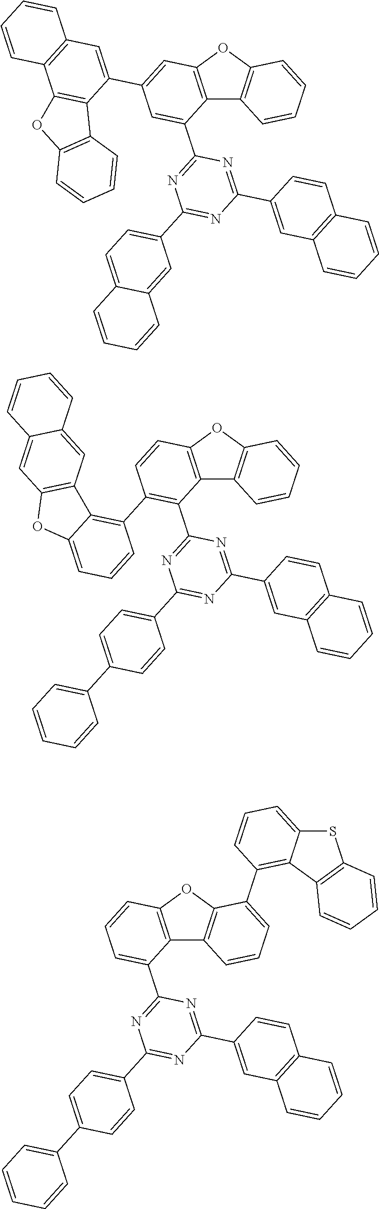 [SMILES: c1ccc2cc(-c3nc(-c4ccc5ccccc5c4)nc(-c4cc(-c5cc6ccccc6c6oc7ccccc7c56)cc5oc6ccccc6c45)n3)ccc2c1 ; c1ccc(-c2ccc(-c3nc(-c4ccc5ccccc5c4)nc(-c4c(-c5cccc6oc7cc8ccccc8cc7c56)ccc5oc6ccccc6c45)n3)cc2)cc1 ; c1ccc(-c2ccc(-c3nc(-c4ccc5ccccc5c4)nc(-c4cccc5oc6c(-c7cccc8sc9ccccc9c78)cccc6c45)n3)cc2)cc1]

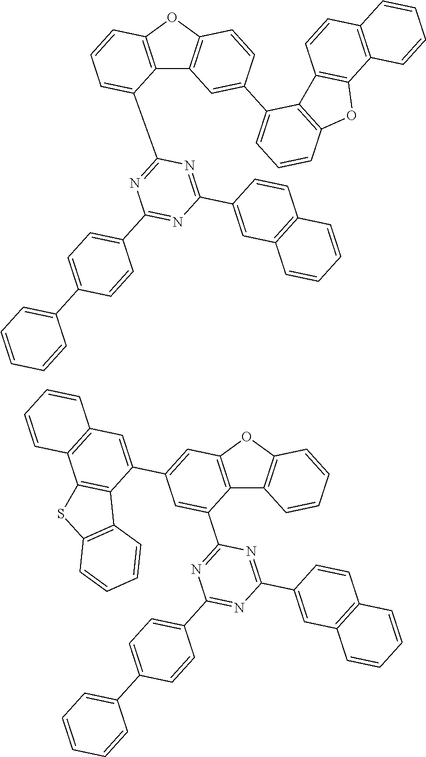

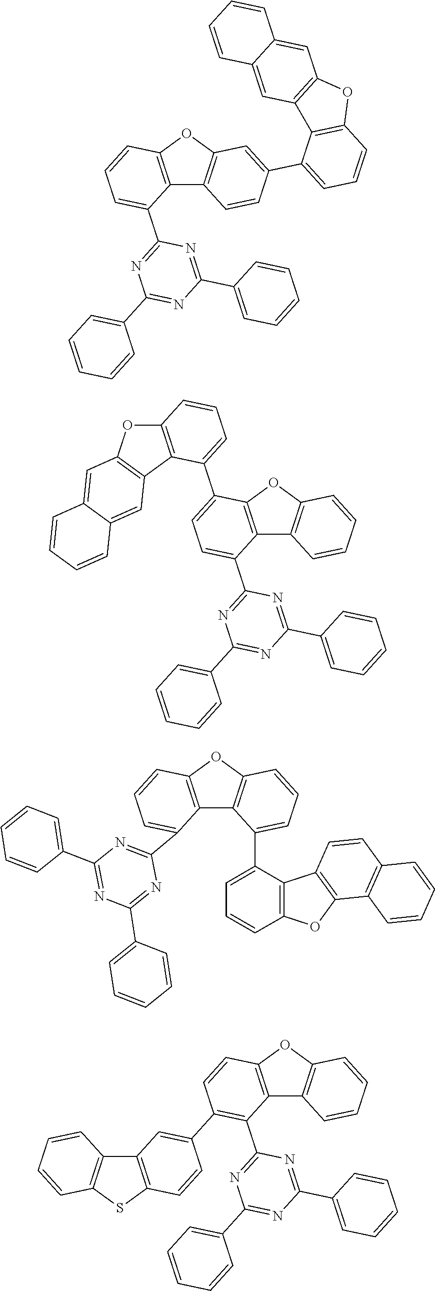

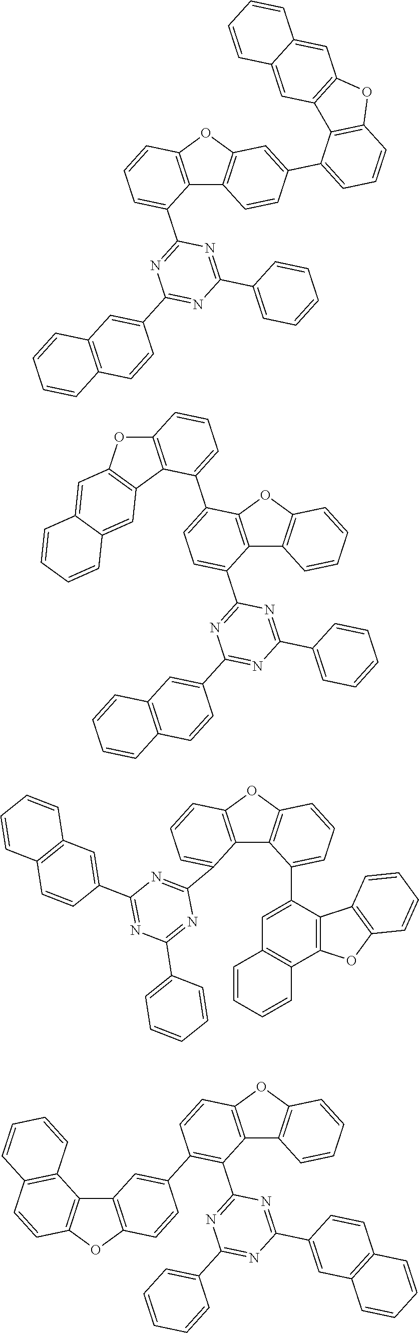 [SMILES: c1ccc(-c2nc(-c3ccc4ccccc4c3)nc(-c3cccc4oc5cc(-c6cccc7oc8cc9ccccc9cc8c67)ccc5c34)n2)cc1 ; c1ccc(-c2nc(-c3ccc4ccccc4c3)nc(-c3ccc(-c4cccc5oc6cc7ccccc7cc6c45)c4oc5ccccc5c34)n2)cc1 ; c1ccc(-c2nc(-c3ccc4ccccc4c3)nc(-c3cccc4oc5cccc(-c6cc7ccccc7c7oc8ccccc8c67)c5c34)n2)cc1 ; c1ccc(-c2nc(-c3ccc4ccccc4c3)nc(-c3c(-c4ccc5oc6ccc7ccccc7c6c5c4)ccc4oc5ccccc5c34)n2)cc1]

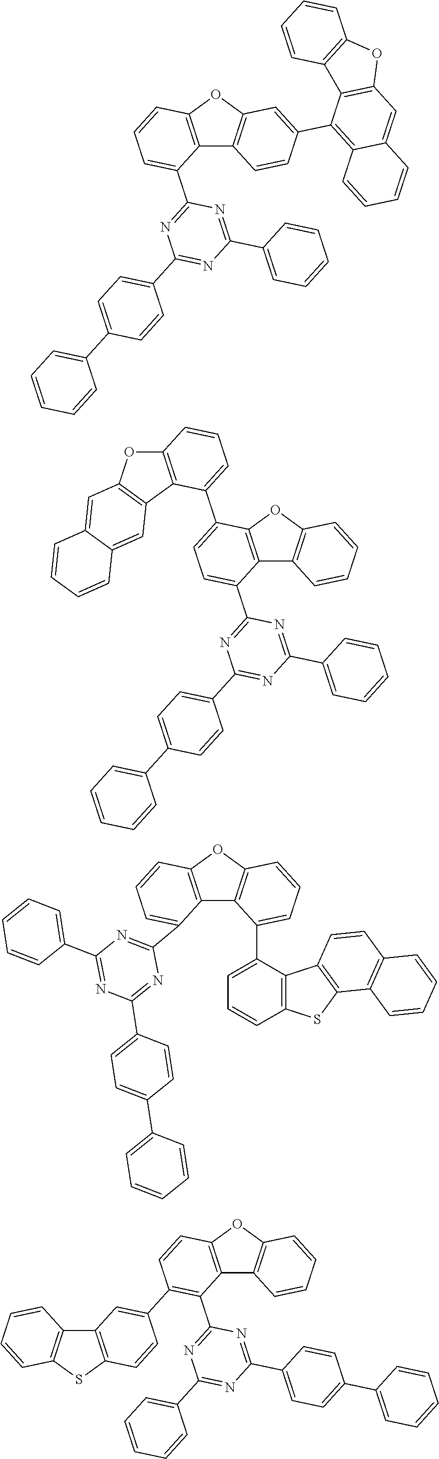

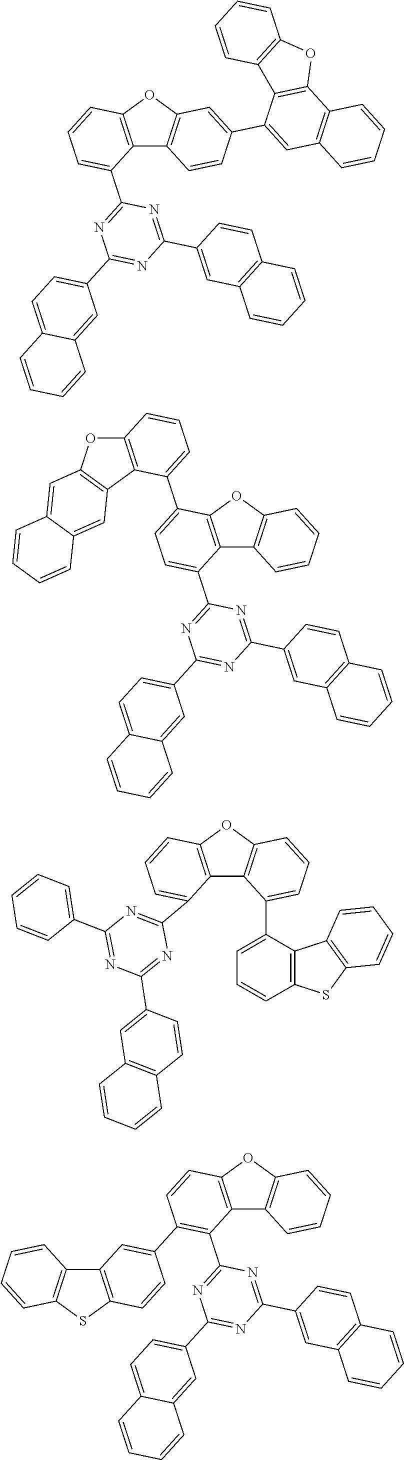

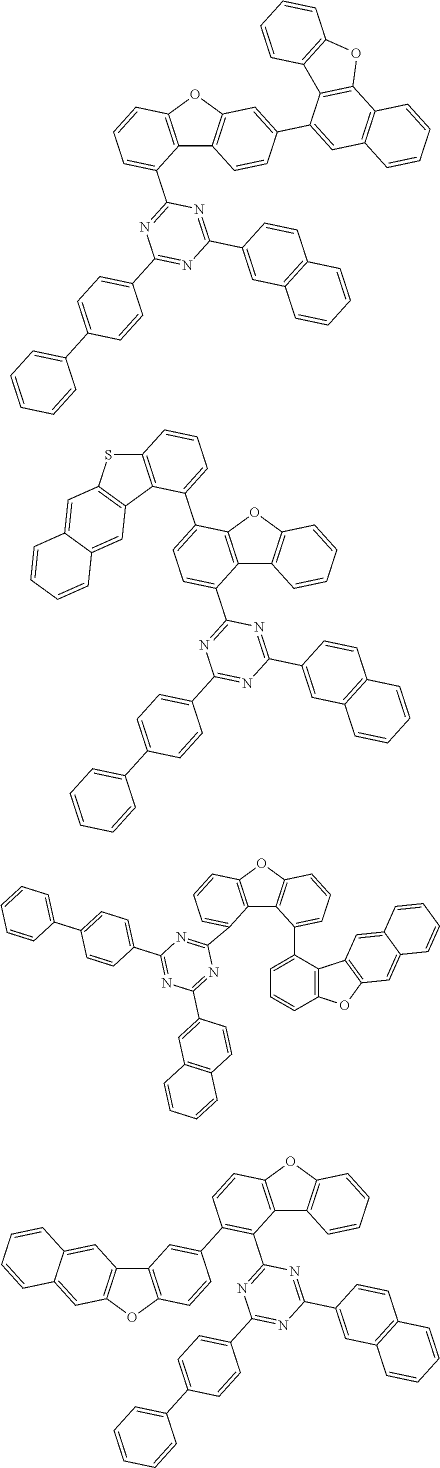 [SMILES: c1ccc(-c2ccc(-c3nc(-c4ccc5ccccc5c4)nc(-c4cccc5oc6cc(-c7cc8ccccc8c8oc9ccccc9c78)ccc6c45)n3)cc2)cc1 ; c1ccc(-c2ccc(-c3nc(-c4ccc5ccccc5c4)nc(-c4ccc(-c5cccc6sc7cc8ccccc8cc7c56)c5oc6ccccc6c45)n3)cc2)cc1 ; c1ccc(-c2ccc(-c3nc(-c4ccc5ccccc5c4)nc(-c4cccc5oc6cccc(-c7cccc8oc9cc%10ccccc%10cc9c78)c6c45)n3)cc2)cc1 ; c1ccc(-c2ccc(-c3nc(-c4ccc5ccccc5c4)nc(-c4c(-c5ccc6oc7cc8ccccc8cc7c6c5)ccc5oc6ccccc6c45)n3)cc2)cc1]

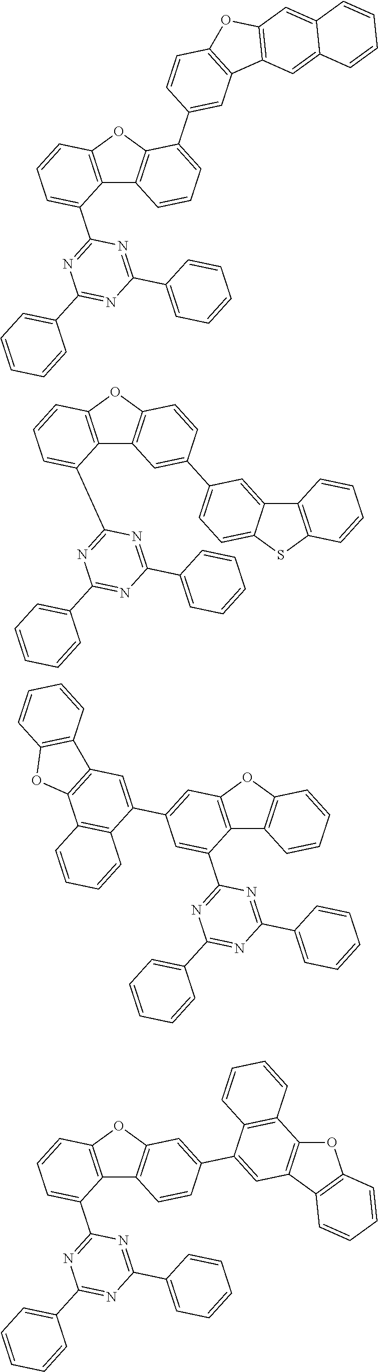

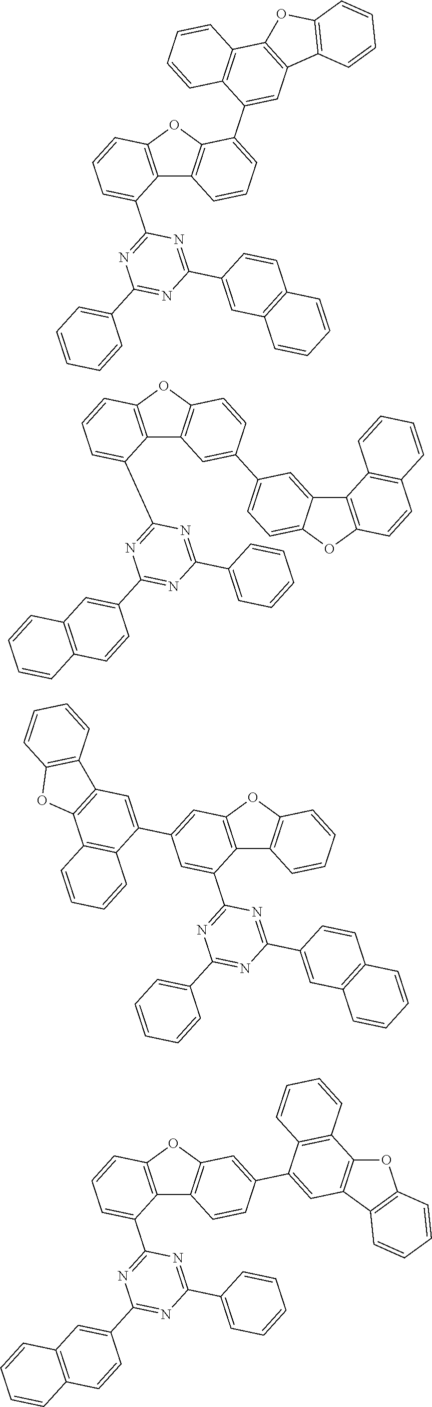

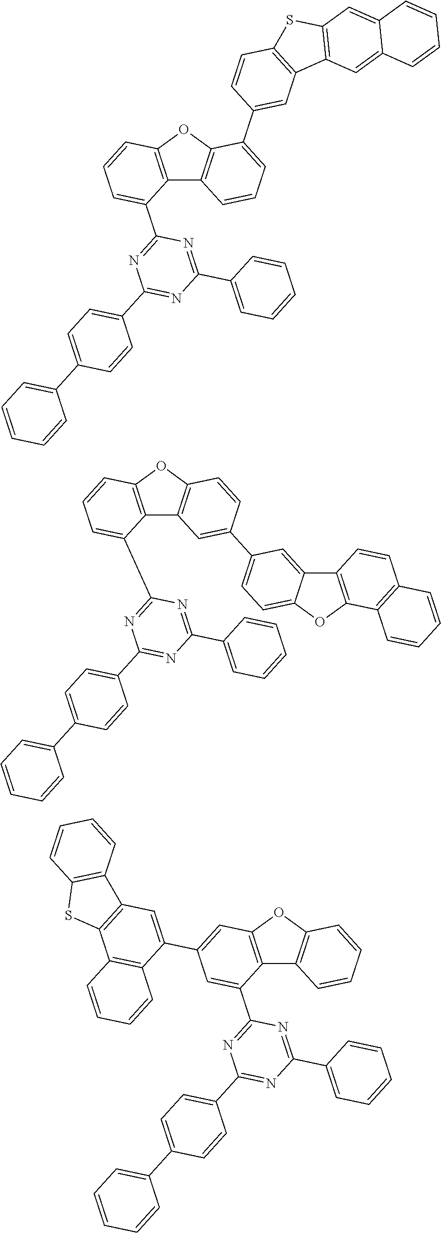

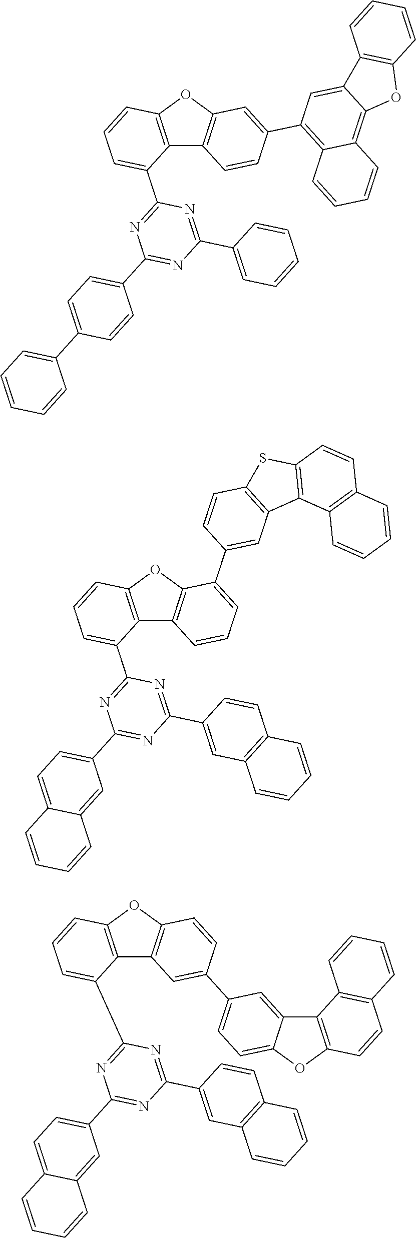 [SMILES: c1ccc(-c2ccc(-c3nc(-c4ccccc4)nc(-c4cccc5oc6cc(-c7cc8c9ccccc9oc8c8ccccc78)ccc6c45)n3)cc2)cc1 ; c1ccc2cc(-c3nc(-c4ccc5ccccc5c4)nc(-c4cccc5oc6c(-c7ccc8sc9ccc%10ccccc%10c9c8c7)cccc6c45)n3)ccc2c1 ; c1ccc2cc(-c3nc(-c4ccc5ccccc5c4)nc(-c4cccc5oc6ccc(-c7ccc8oc9ccc%10ccccc%10c9c8c7)cc6c45)n3)ccc2c1]

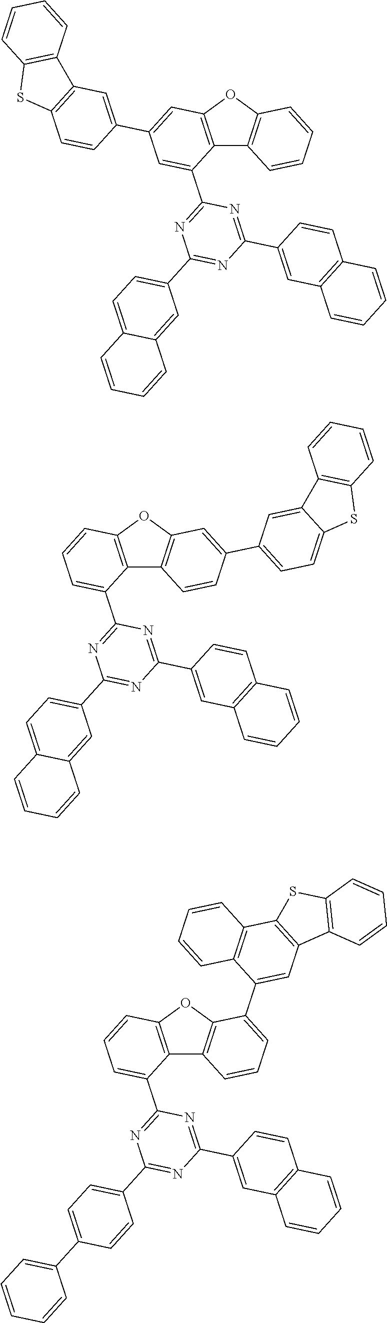 [SMILES: c1ccc2cc(-c3nc(-c4ccc5ccccc5c4)nc(-c4cc(-c5ccc6sc7ccccc7c6c5)cc5oc6ccccc6c45)n3)ccc2c1 ; c1ccc2cc(-c3nc(-c4ccc5ccccc5c4)nc(-c4cccc5oc6cc(-c7ccc8sc9ccccc9c8c7)ccc6c45)n3)ccc2c1 ; c1ccc(-c2ccc(-c3nc(-c4ccc5ccccc5c4)nc(-c4cccc5oc6c(-c7cc8c9ccccc9sc8c8ccccc78)cccc6c45)n3)cc2)cc1]

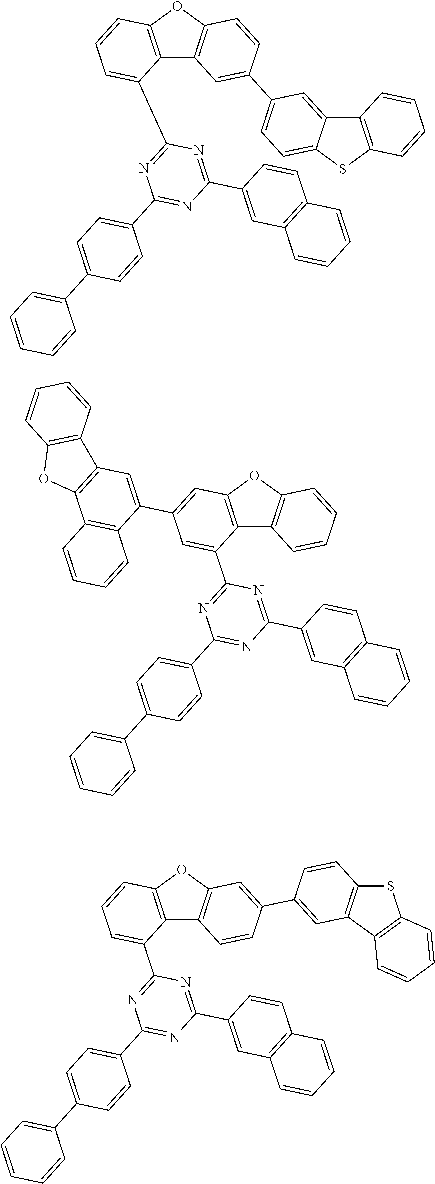

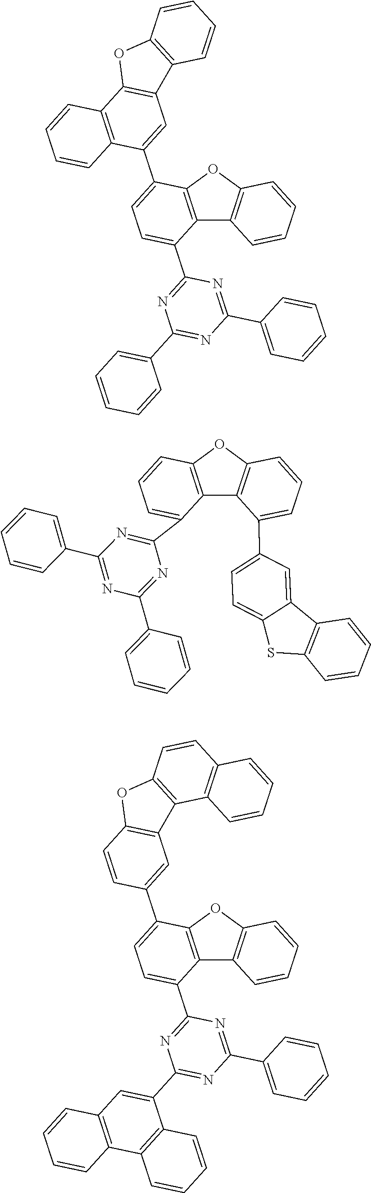 [SMILES: c1ccc(-c2nc(-c3ccccc3)nc(-c3ccc(-c4cc5c6ccccc6oc5c5ccccc45)c4oc5ccccc5c34)n2)cc1 ; c1ccc(-c2nc(-c3ccccc3)nc(-c3cccc4oc5cccc(-c6ccc7sc8ccccc8c7c6)c5c34)n2)cc1 ; c1ccc(-c2nc(-c3cc4ccccc4c4ccccc34)nc(-c3ccc(-c4ccc5oc6ccc7ccccc7c6c5c4)c4oc5ccccc5c34)n2)cc1]

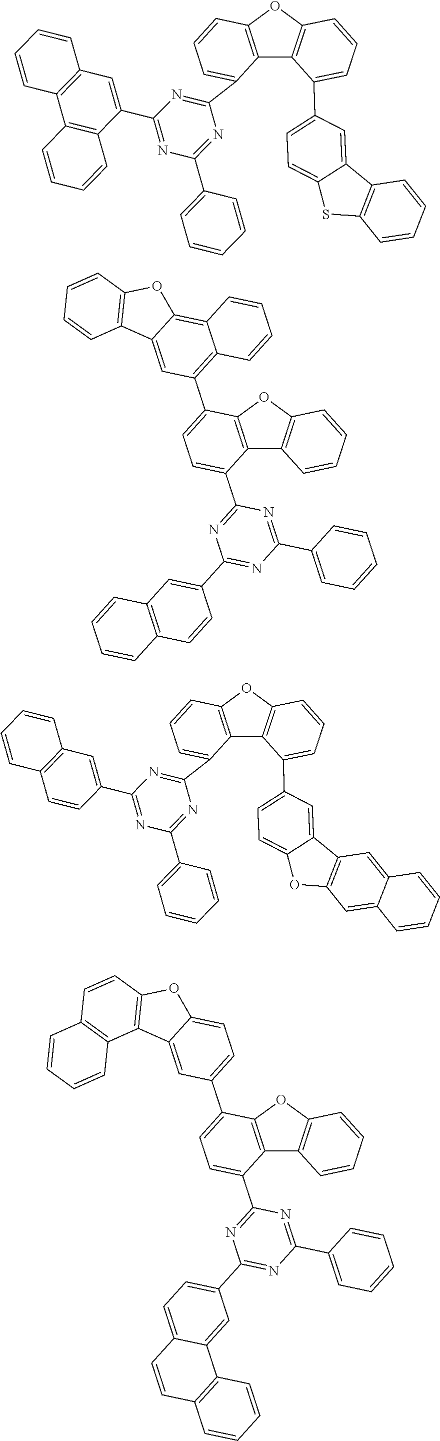

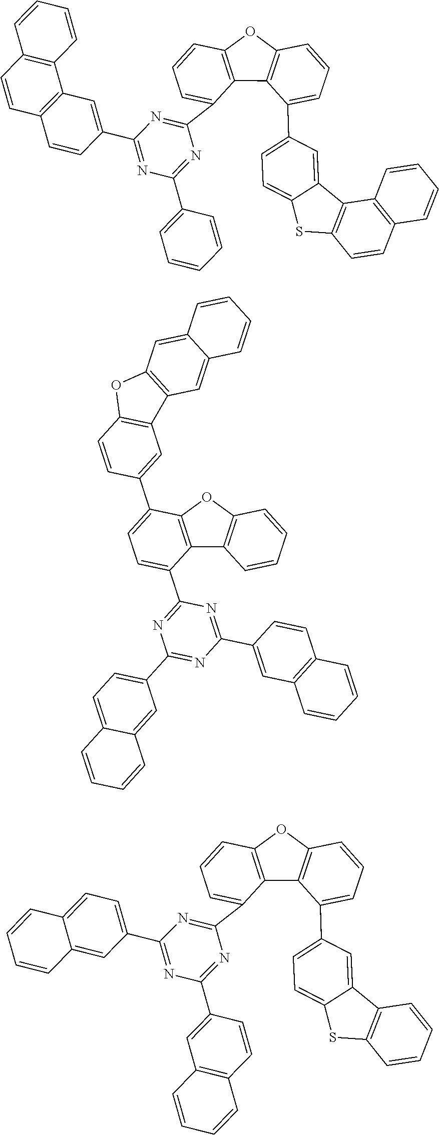

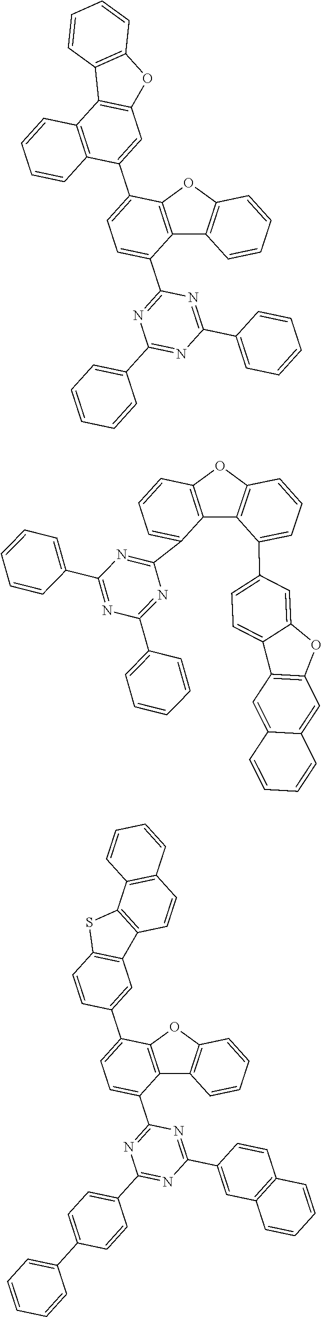 [SMILES: c1ccc(-c2nc(-c3ccccc3)nc(-c3ccc(-c4cc5oc6ccccc6c5c5ccccc45)c4oc5ccccc5c34)n2)cc1 ; c1ccc(-c2nc(-c3ccccc3)nc(-c3cccc4oc5cccc(-c6ccc7c(c6)oc6cc8ccccc8cc67)c5c34)n2)cc1 ; c1ccc(-c2ccc(-c3nc(-c4ccc5ccccc5c4)nc(-c4ccc(-c5ccc6sc7c8ccccc8ccc7c6c5)c5oc6ccccc6c45)n3)cc2)cc1]

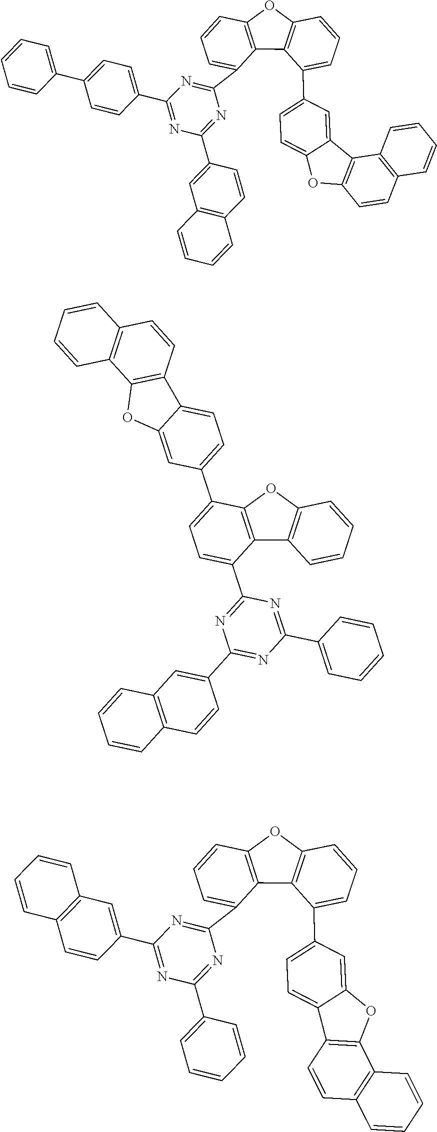 [SMILES: c1ccc(-c2ccc(-c3nc(-c4ccc5ccccc5c4)nc(-c4cccc5oc6cccc(-c7ccc8oc9ccc%10ccccc%10c9c8c7)c6c45)n3)cc2)cc1 ; c1ccc(-c2nc(-c3ccc4ccccc4c3)nc(-c3ccc(-c4ccc5c(c4)oc4c6ccccc6ccc54)c4oc5ccccc5c34)n2)cc1 ; c1ccc(-c2nc(-c3ccc4ccccc4c3)nc(-c3cccc4oc5cccc(-c6ccc7c(c6)oc6c8ccccc8ccc76)c5c34)n2)cc1]

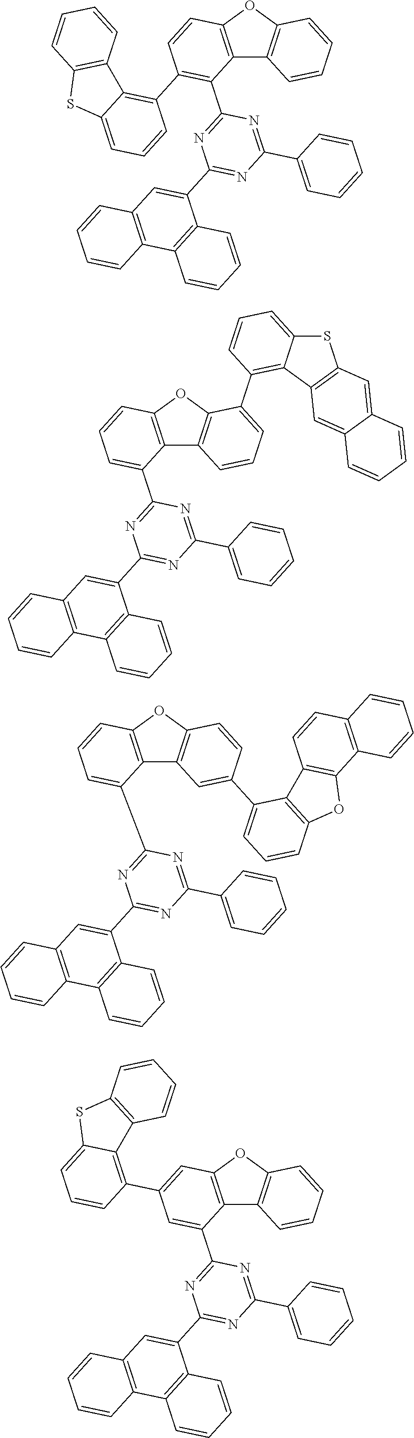

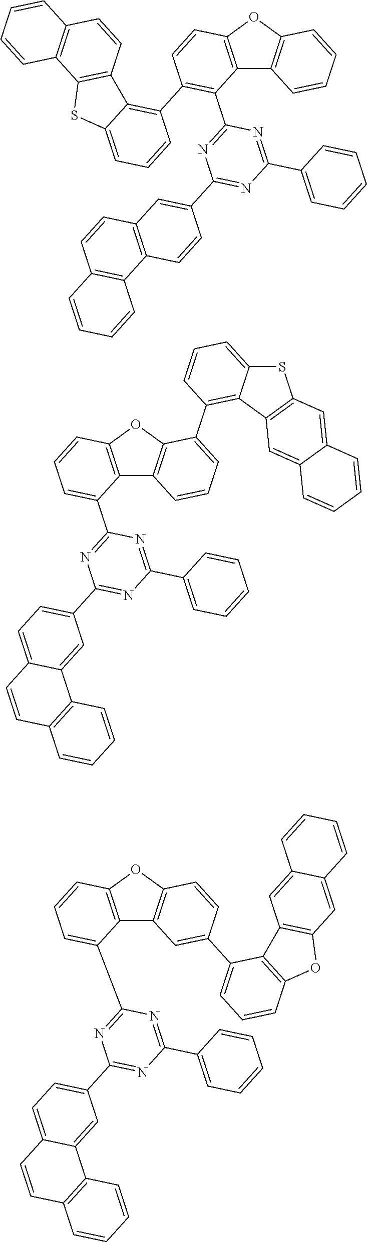 [SMILES: c1ccc(-c2nc(-c3ccc4c(ccc5ccccc54)c3)nc(-c3c(-c4cccc5sc6c7ccccc7ccc6c45)ccc4oc5ccccc5c34)n2)cc1 ; c1ccc(-c2nc(-c3ccc4ccc5ccccc5c4c3)nc(-c3cccc4oc5c(-c6cccc7sc8cc9ccccc9cc8c67)cccc5c34)n2)cc1 ; c1ccc(-c2nc(-c3ccc4ccc5ccccc5c4c3)nc(-c3cccc4oc5ccc(-c6cccc7oc8cc9ccccc9cc8c67)cc5c34)n2)cc1]

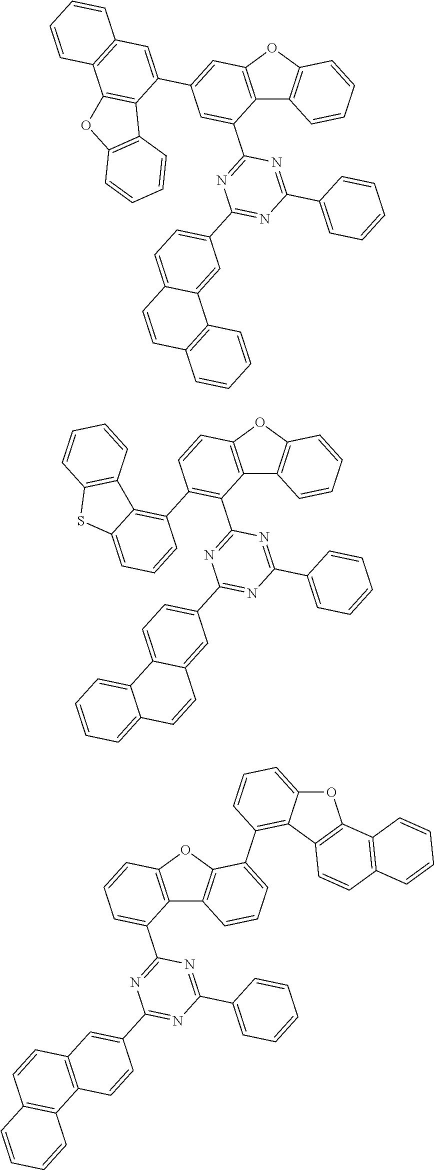

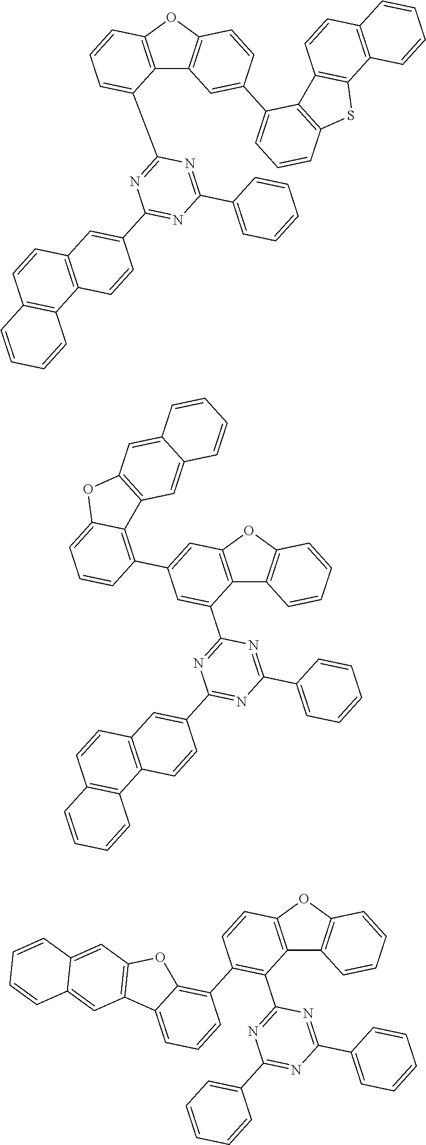 [SMILES: c1ccc(-c2nc(-c3ccc4c(ccc5ccccc54)c3)nc(-c3cccc4oc5ccc(-c6cccc7sc8c9ccccc9ccc8c67)cc5c34)n2)cc1 ; c1ccc(-c2nc(-c3ccc4c(ccc5ccccc54)c3)nc(-c3cc(-c4cccc5oc6cc7ccccc7cc6c45)cc4oc5ccccc5c34)n2)cc1 ; c1ccc(-c2nc(-c3ccccc3)nc(-c3c(-c4cccc5c4oc4cc6ccccc6cc45)ccc4oc5ccccc5c34)n2)cc1]

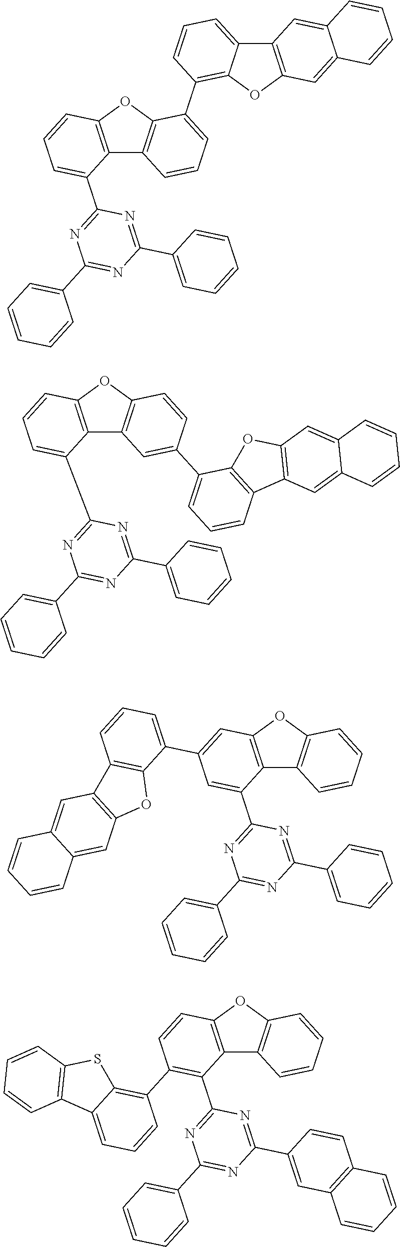 [SMILES: c1ccc(-c2nc(-c3ccccc3)nc(-c3cccc4oc5c(-c6cccc7c6oc6cc8ccccc8cc67)cccc5c34)n2)cc1 ; c1ccc(-c2nc(-c3ccccc3)nc(-c3cccc4oc5ccc(-c6cccc7c6oc6cc8ccccc8cc67)cc5c34)n2)cc1 ; c1ccc(-c2nc(-c3ccccc3)nc(-c3cc(-c4cccc5c4oc4cc6ccccc6cc45)cc4oc5ccccc5c34)n2)cc1 ; c1ccc(-c2nc(-c3ccc4ccccc4c3)nc(-c3c(-c4cccc5c4sc4ccccc45)ccc4oc5ccccc5c34)n2)cc1]

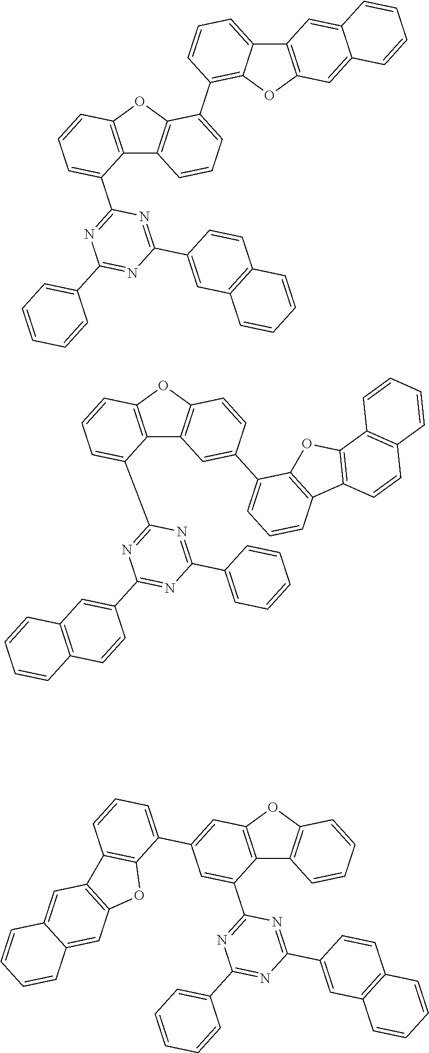 [SMILES: c1ccc(-c2nc(-c3ccc4ccccc4c3)nc(-c3cccc4oc5c(-c6cccc7c6oc6cc8ccccc8cc67)cccc5c34)n2)cc1 ; c1ccc(-c2nc(-c3ccc4ccccc4c3)nc(-c3cccc4oc5ccc(-c6cccc7c6oc6c8ccccc8ccc76)cc5c34)n2)cc1 ; c1ccc(-c2nc(-c3ccc4ccccc4c3)nc(-c3cc(-c4cccc5c4oc4cc6ccccc6cc45)cc4oc5ccccc5c34)n2)cc1]

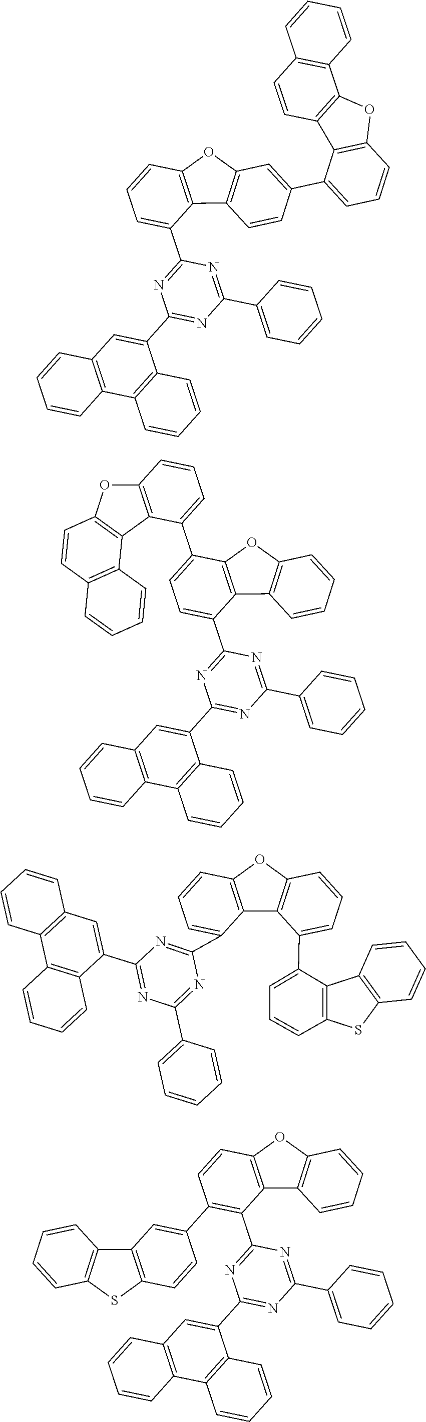

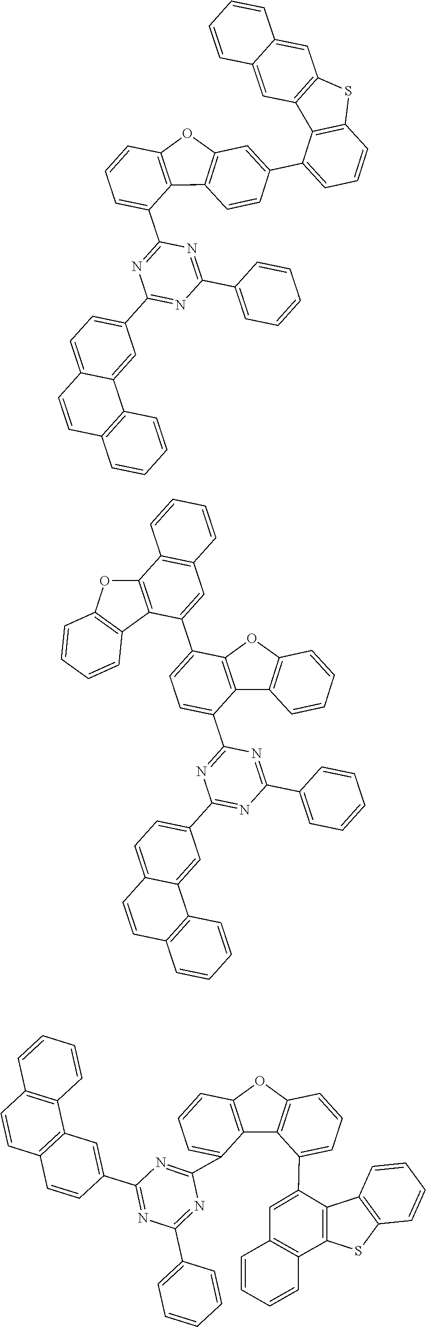

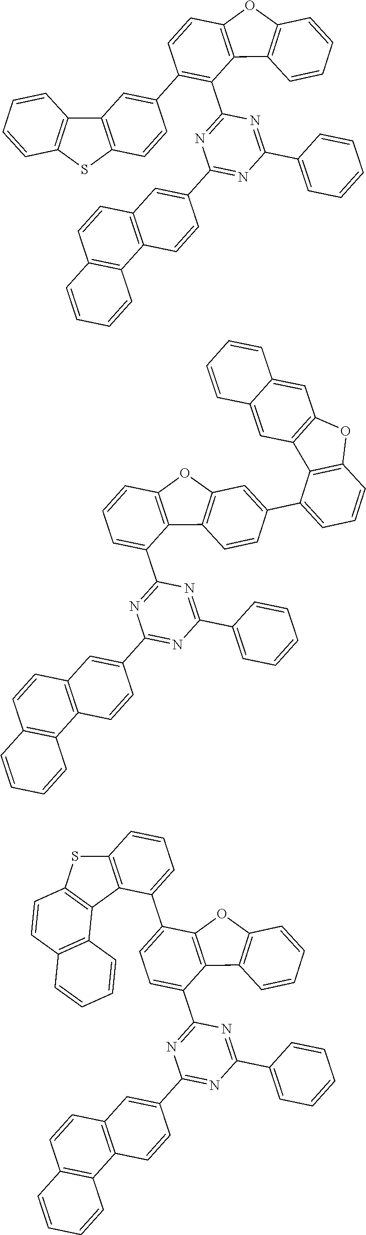 [SMILES: c1ccc(-c2nc(-c3ccc4c(ccc5ccccc54)c3)nc(-c3c(-c4ccc5sc6ccccc6c5c4)ccc4oc5ccccc5c34)n2)cc1 ; c1ccc(-c2nc(-c3ccc4c(ccc5ccccc54)c3)nc(-c3cccc4oc5cc(-c6cccc7oc8cc9ccccc9cc8c67)ccc5c34)n2)cc1 ; c1ccc(-c2nc(-c3ccc4c(ccc5ccccc54)c3)nc(-c3ccc(-c4cccc5sc6ccc7ccccc7c6c45)c4oc5ccccc5c34)n2)cc1]

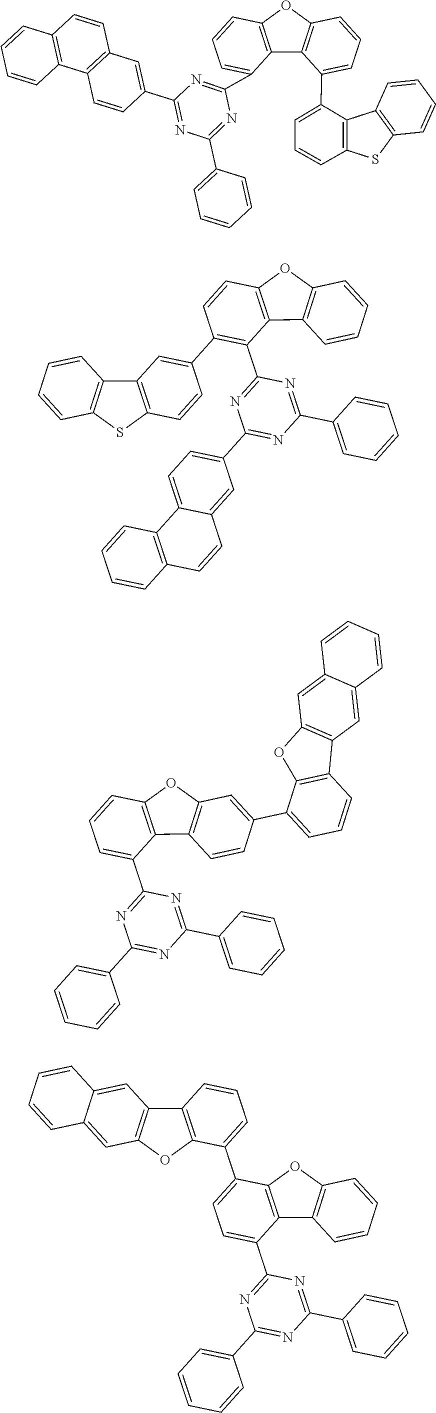

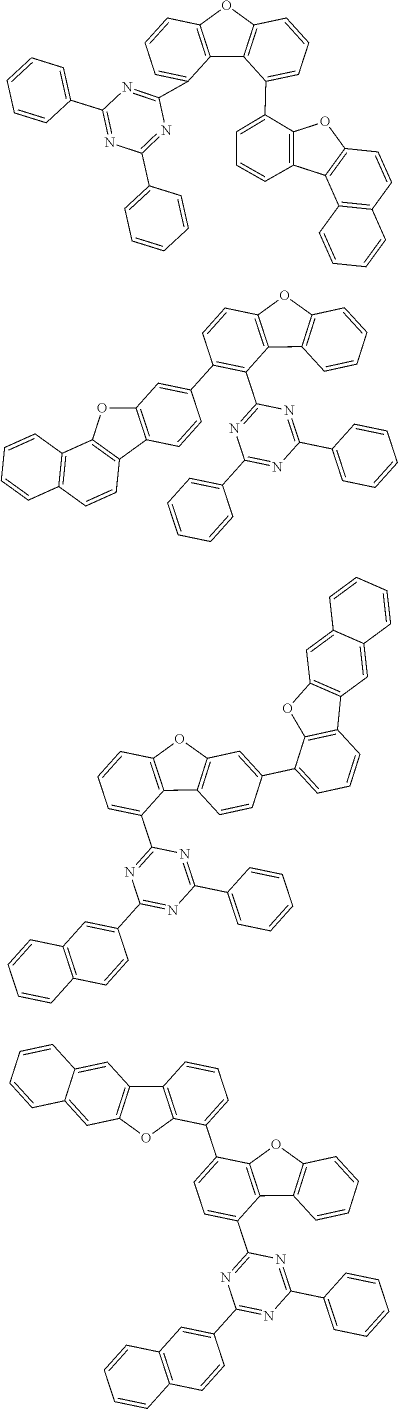

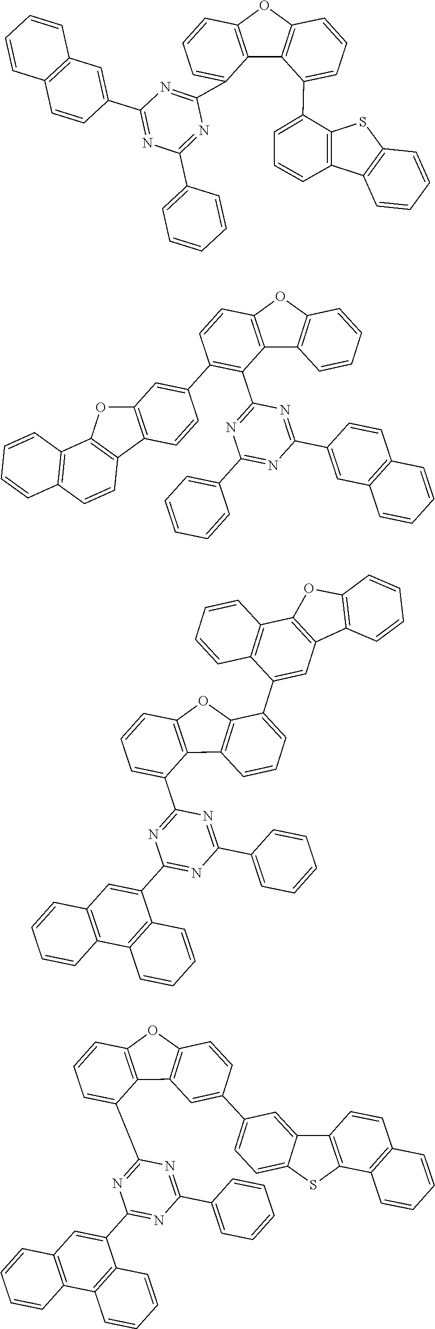 [SMILES: c1ccc(-c2nc(-c3ccc4ccccc4c3)nc(-c3cccc4oc5cccc(-c6cccc7c6sc6ccccc67)c5c34)n2)cc1 ; c1ccc(-c2nc(-c3ccc4ccccc4c3)nc(-c3c(-c4ccc5c(c4)oc4c6ccccc6ccc54)ccc4oc5ccccc5c34)n2)cc1 ; c1ccc(-c2nc(-c3cc4ccccc4c4ccccc34)nc(-c3cccc4oc5c(-c6cc7c8ccccc8oc7c7ccccc67)cccc5c34)n2)cc1 ; c1ccc(-c2nc(-c3cc4ccccc4c4ccccc34)nc(-c3cccc4oc5ccc(-c6ccc7sc8c9ccccc9ccc8c7c6)cc5c34)n2)cc1]

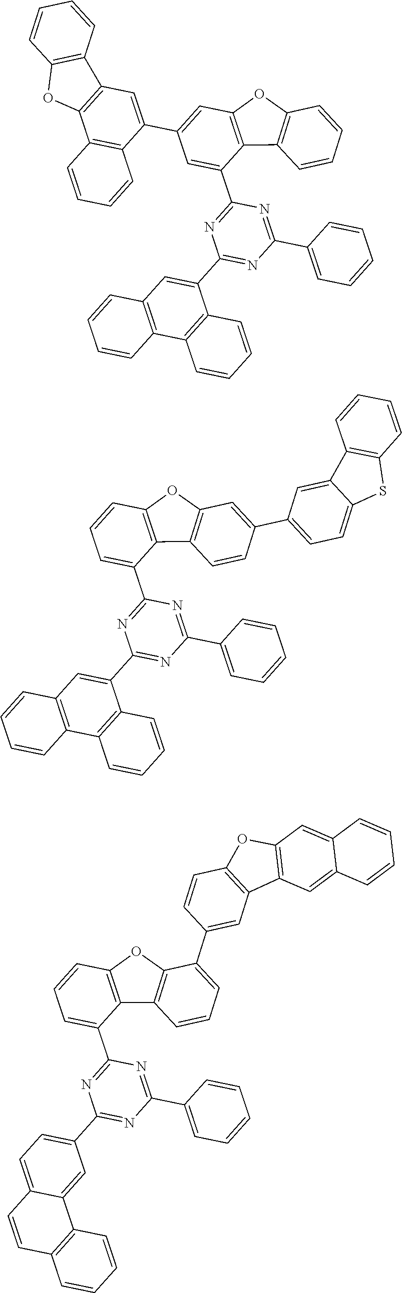 [SMILES: c1ccc(-c2nc(-c3cc4ccccc4c4ccccc34)nc(-c3cc(-c4cc5c6ccccc6oc5c5ccccc45)cc4oc5ccccc5c34)n2)cc1 ; c1ccc(-c2nc(-c3cc4ccccc4c4ccccc34)nc(-c3cccc4oc5cc(-c6ccc7sc8ccccc8c7c6)ccc5c34)n2)cc1 ; c1ccc(-c2nc(-c3ccc4ccc5ccccc5c4c3)nc(-c3cccc4oc5c(-c6ccc7oc8cc9ccccc9cc8c7c6)cccc5c34)n2)cc1]

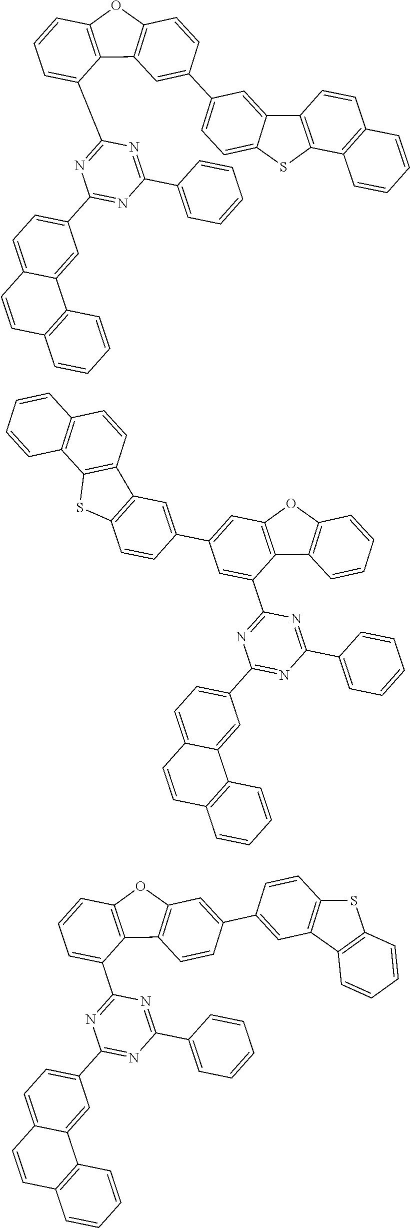

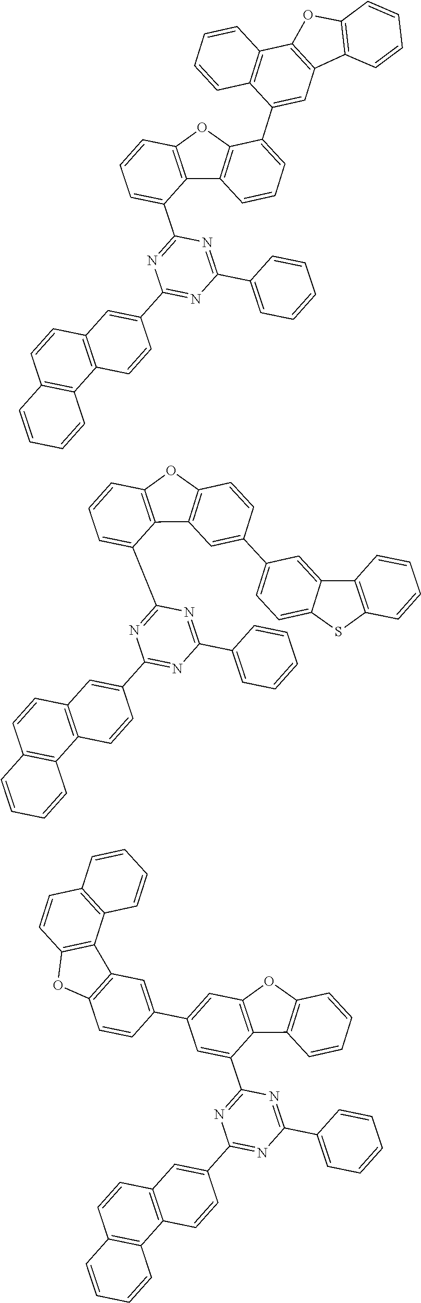 [SMILES: c1ccc(-c2nc(-c3ccc4c(ccc5ccccc54)c3)nc(-c3cccc4oc5c(-c6cc7c8ccccc8oc7c7ccccc67)cccc5c34)n2)cc1 ; c1ccc(-c2nc(-c3ccc4c(ccc5ccccc54)c3)nc(-c3cccc4oc5ccc(-c6ccc7sc8ccccc8c7c6)cc5c34)n2)cc1 ; c1ccc(-c2nc(-c3ccc4c(ccc5ccccc54)c3)nc(-c3cc(-c4ccc5oc6ccc7ccccc7c6c5c4)cc4oc5ccccc5c34)n2)cc1]

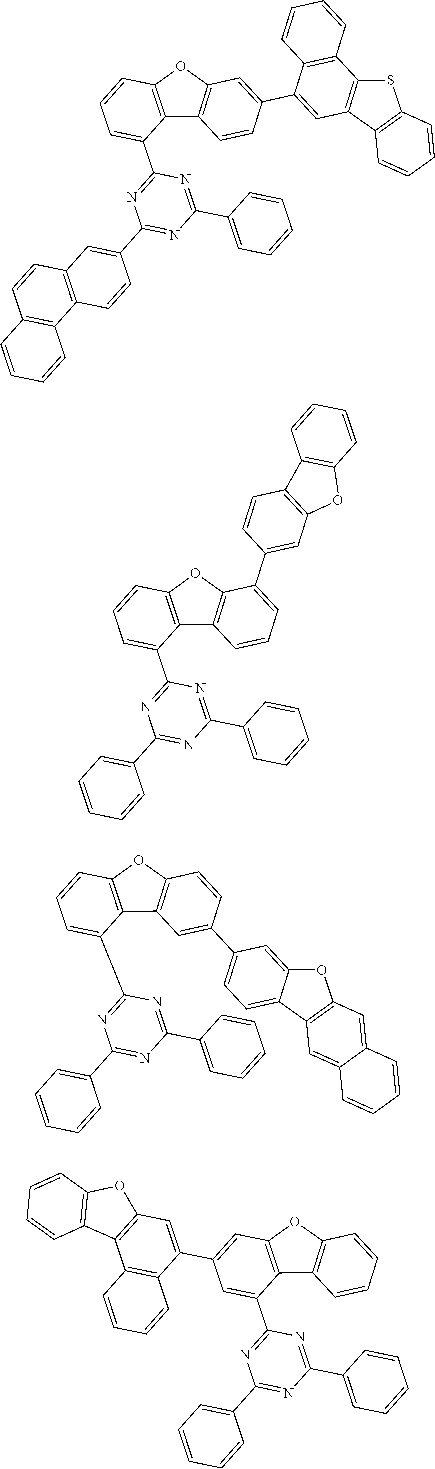

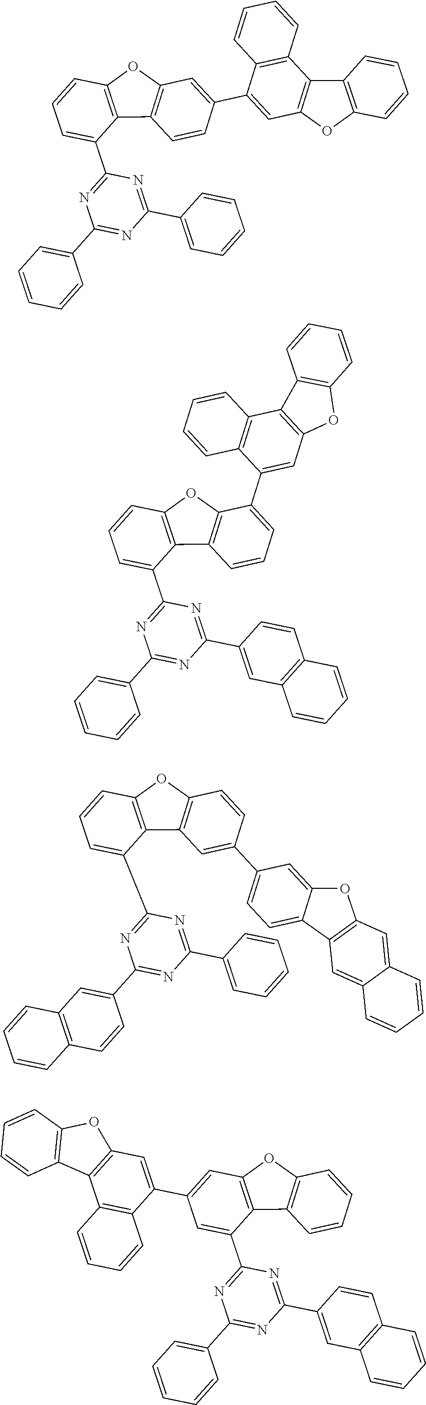

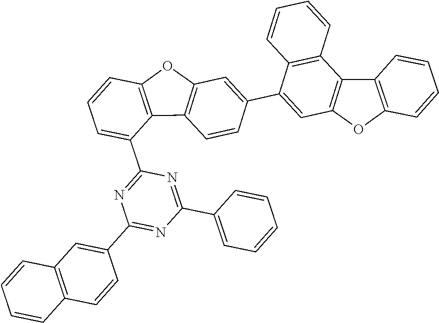 [SMILES: c1ccc(-c2nc(-c3ccc4ccccc4c3)nc(-c3cccc4oc5cc(-c6cc7oc8ccccc8c7c7ccccc67)ccc5c34)n2)cc1]

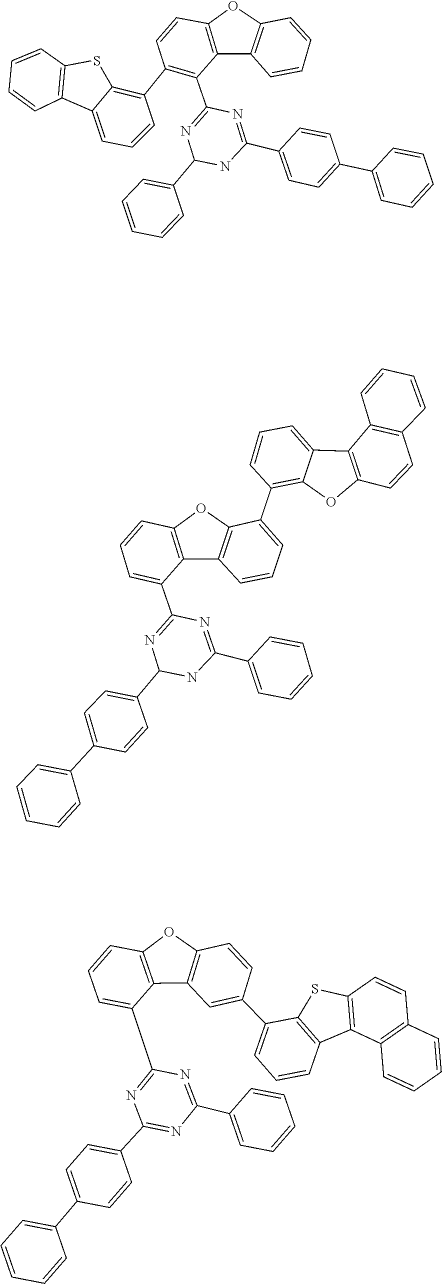 [SMILES: c1ccc(-c2ccc(C3=NC(c4c(-c5cccc6c5sc5ccccc56)ccc5oc6ccccc6c45)=NC(c4ccccc4)N3)cc2)cc1 ; c1ccc(C2=NC(c3cccc4oc5c(-c6cccc7c6oc6ccc8ccccc8c67)cccc5c34)=NC(c3ccc(-c4ccccc4)cc3)N2)cc1 ; c1ccc(-c2ccc(-c3nc(-c4ccccc4)nc(-c4cccc5oc6ccc(-c7cccc8c7sc7ccc9ccccc9c78)cc6c45)n3)cc2)cc1]

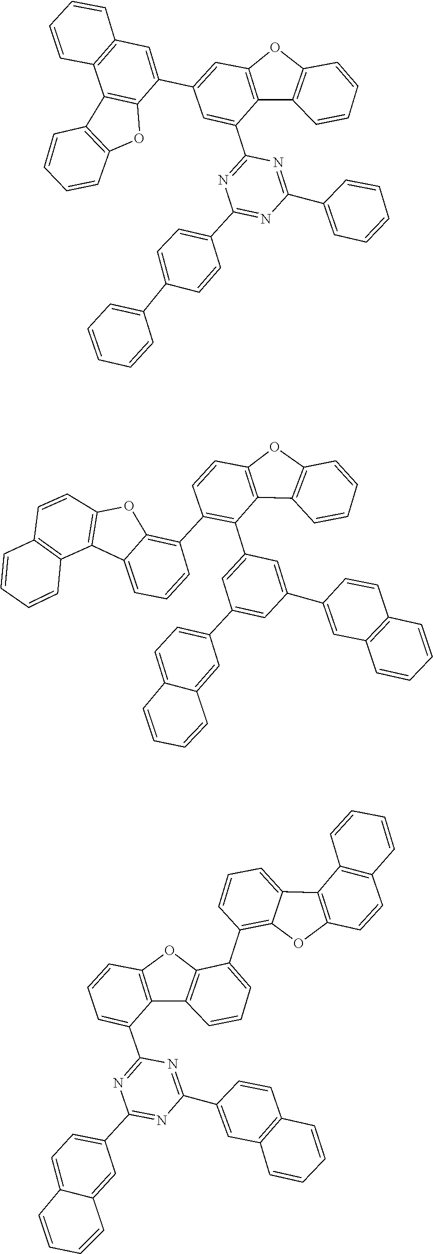 [SMILES: c1ccc(-c2ccc(-c3nc(-c4ccccc4)nc(-c4cc(-c5cc6ccccc6c6c5oc5ccccc56)cc5oc6ccccc6c45)n3)cc2)cc1 ; c1ccc2cc(-c3cc(-c4ccc5ccccc5c4)cc(-c4c(-c5cccc6c5oc5ccc7ccccc7c56)ccc5oc6ccccc6c45)c3)ccc2c1 ; c1ccc2cc(-c3nc(-c4ccc5ccccc5c4)nc(-c4cccc5oc6c(-c7cccc8c7oc7ccc9ccccc9c78)cccc6c45)n3)ccc2c1]

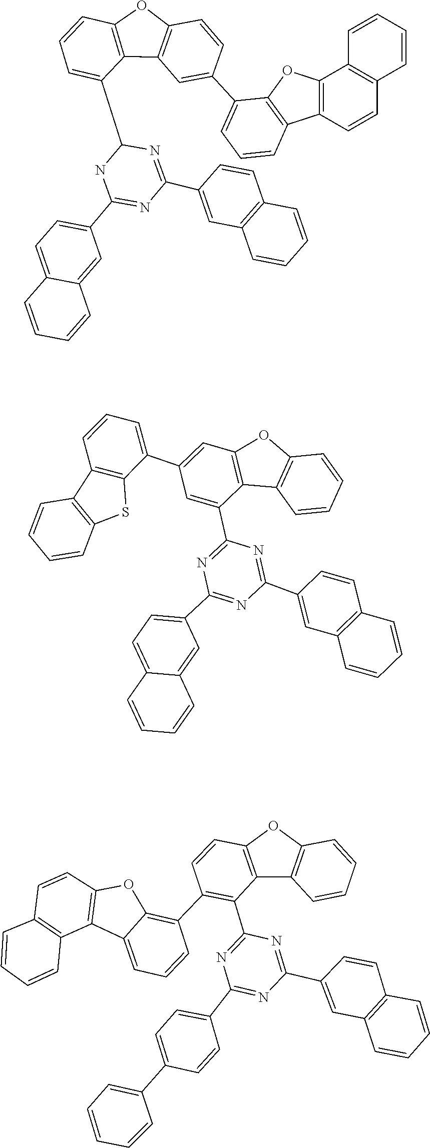

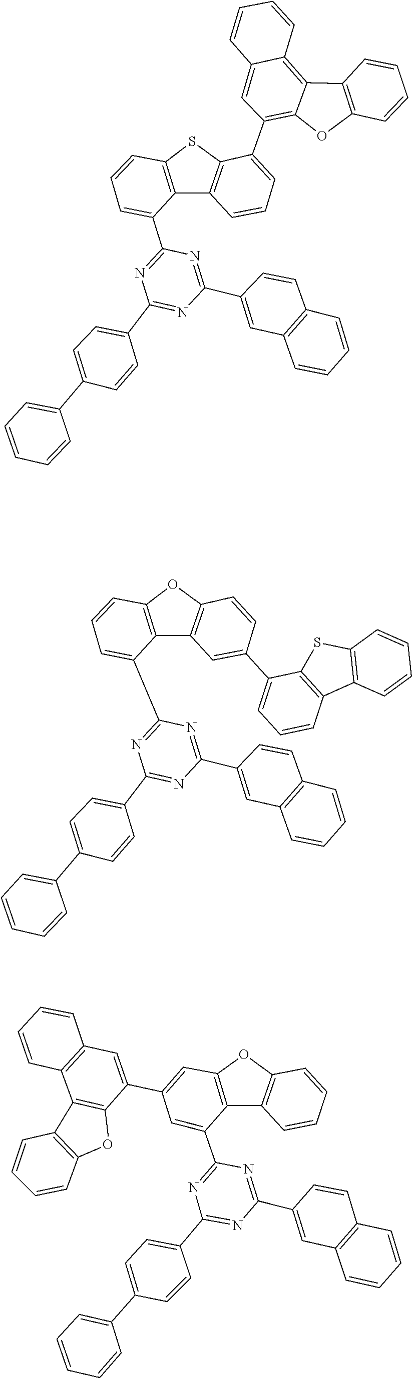 [SMILES: c1ccc(-c2ccc(-c3nc(-c4ccc5ccccc5c4)nc(-c4cccc5sc6c(-c7cc8ccccc8c8c7oc7ccccc78)cccc6c45)n3)cc2)cc1 ; c1ccc(-c2ccc(-c3nc(-c4ccc5ccccc5c4)nc(-c4cccc5oc6ccc(-c7cccc8c7sc7ccccc78)cc6c45)n3)cc2)cc1 ; c1ccc(-c2ccc(-c3nc(-c4ccc5ccccc5c4)nc(-c4cc(-c5cc6ccccc6c6c5oc5ccccc56)cc5oc6ccccc6c45)n3)cc2)cc1]

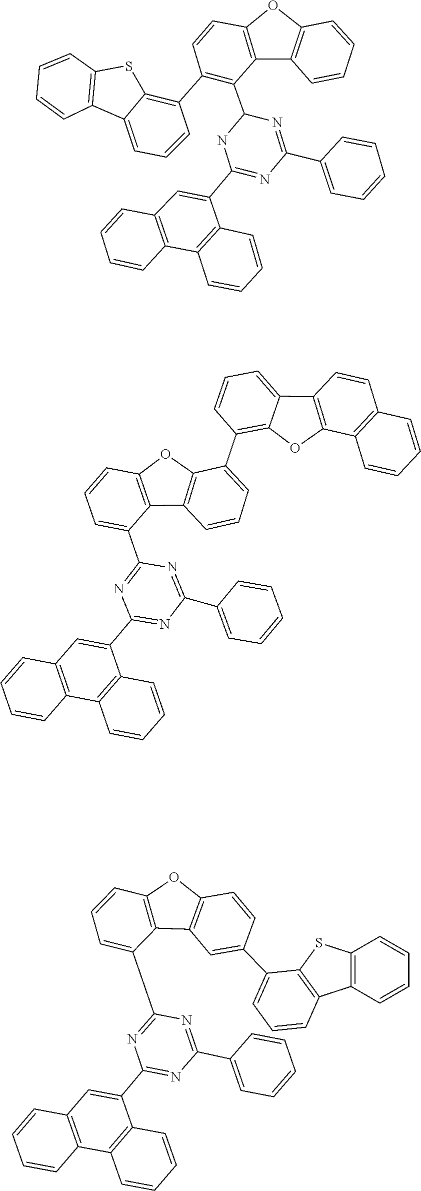

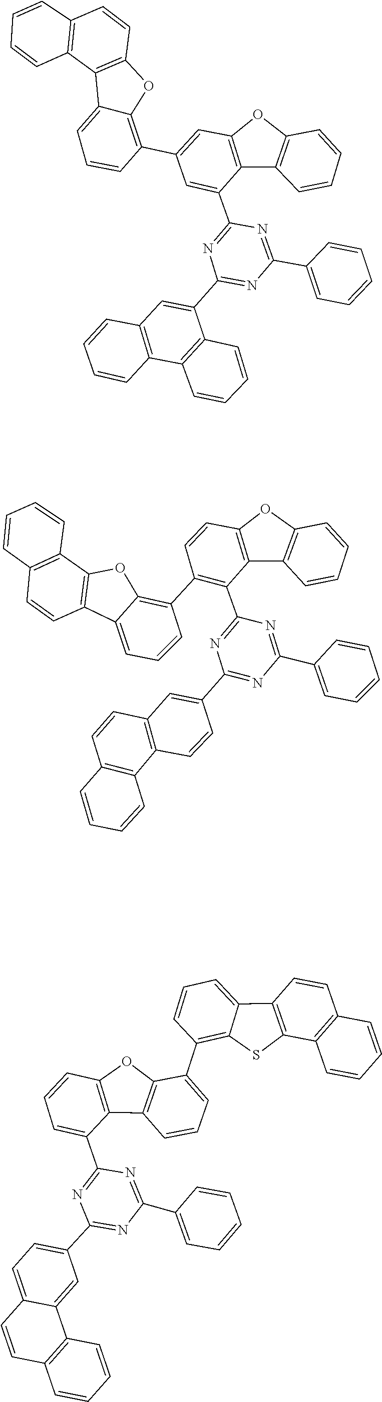 [SMILES: c1ccc(-c2nc(-c3cc4ccccc4c4ccccc34)nc(-c3cc(-c4cccc5c4oc4ccc6ccccc6c45)cc4oc5ccccc5c34)n2)cc1 ; c1ccc(-c2nc(-c3ccc4c(ccc5ccccc54)c3)nc(-c3c(-c4cccc5c4oc4c6ccccc6ccc54)ccc4oc5ccccc5c34)n2)cc1 ; c1ccc(-c2nc(-c3ccc4ccc5ccccc5c4c3)nc(-c3cccc4oc5c(-c6cccc7c6sc6c8ccccc8ccc76)cccc5c34)n2)cc1]

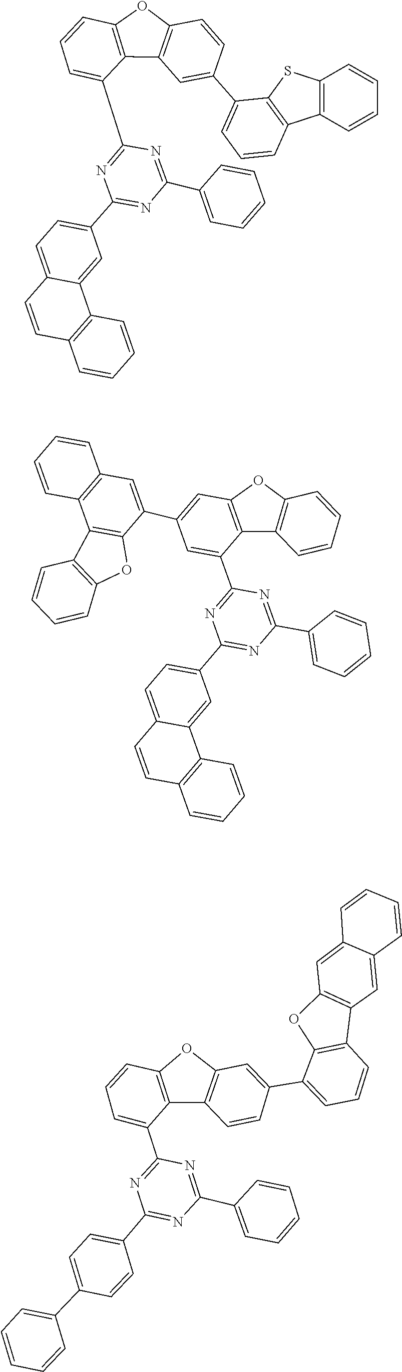 [SMILES: c1ccc(-c2nc(-c3ccc4ccc5ccccc5c4c3)nc(-c3cccc4oc5ccc(-c6cccc7c6sc6ccccc67)cc5c34)n2)cc1 ; c1ccc(-c2nc(-c3ccc4ccc5ccccc5c4c3)nc(-c3cc(-c4cc5ccccc5c5c4oc4ccccc45)cc4oc5ccccc5c34)n2)cc1 ; c1ccc(-c2ccc(-c3nc(-c4ccccc4)nc(-c4cccc5oc6cc(-c7cccc8c7oc7cc9ccccc9cc78)ccc6c45)n3)cc2)cc1]

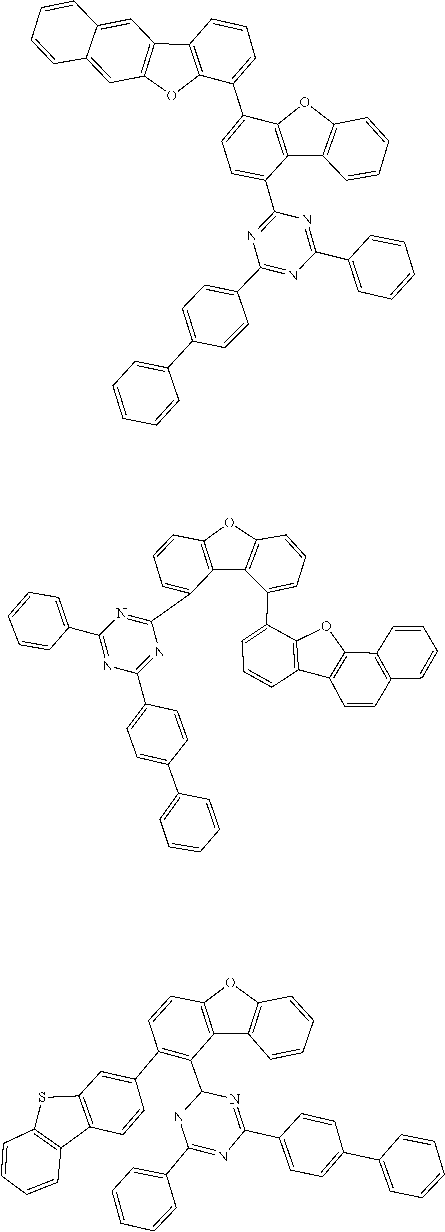

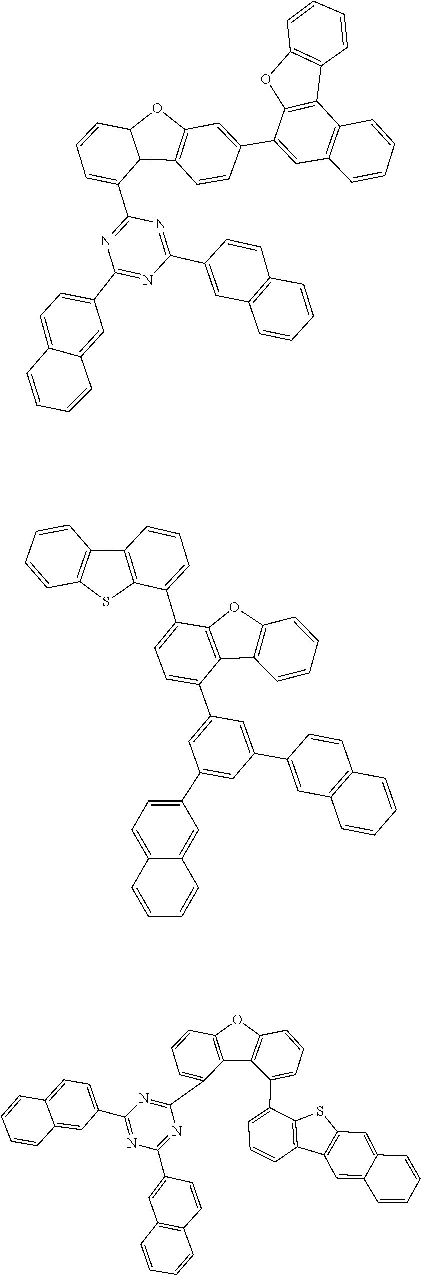 [SMILES: C1=CC2Oc3cc(-c4cc5ccccc5c5c4oc4ccccc45)ccc3C2C(c2nc(-c3ccc4ccccc4c3)nc(-c3ccc4ccccc4c3)n2)=C1 ; c1ccc2cc(-c3cc(-c4ccc5ccccc5c4)cc(-c4ccc(-c5cccc6c5sc5ccccc56)c5oc6ccccc6c45)c3)ccc2c1 ; c1ccc2cc(-c3nc(-c4ccc5ccccc5c4)nc(-c4cccc5oc6cccc(-c7cccc8c7sc7cc9ccccc9cc78)c6c45)n3)ccc2c1]

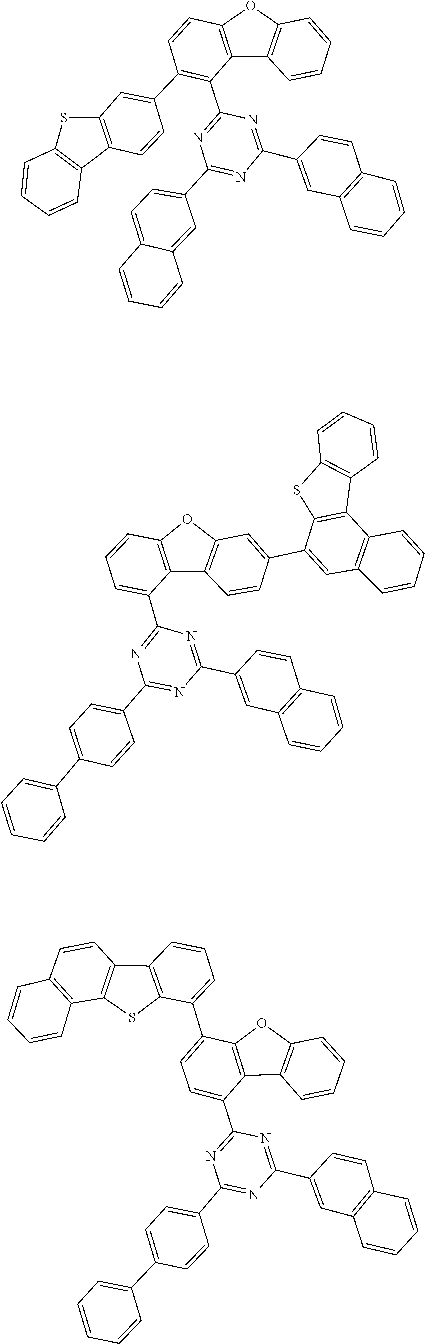 [SMILES: c1ccc2cc(-c3nc(-c4ccc5ccccc5c4)nc(-c4c(-c5ccc6c(c5)sc5ccccc56)ccc5oc6ccccc6c45)n3)ccc2c1 ; c1ccc(-c2ccc(-c3nc(-c4ccc5ccccc5c4)nc(-c4cccc5oc6cc(-c7cc8ccccc8c8c7sc7ccccc78)ccc6c45)n3)cc2)cc1 ; c1ccc(-c2ccc(-c3nc(-c4ccc5ccccc5c4)nc(-c4ccc(-c5cccc6c5sc5c7ccccc7ccc65)c5oc6ccccc6c45)n3)cc2)cc1]

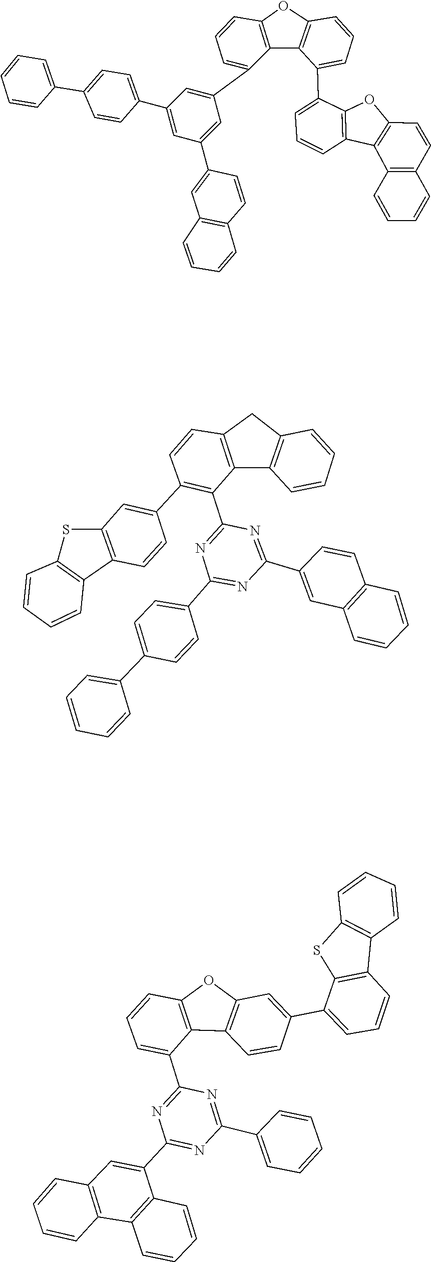 [SMILES: c1ccc(-c2ccc(-c3cc(-c4ccc5ccccc5c4)cc(-c4cccc5oc6cccc(-c7cccc8c7oc7ccc9ccccc9c78)c6c45)c3)cc2)cc1 ; c1ccc(-c2ccc(-c3nc(-c4ccc5ccccc5c4)nc(-c4c(-c5ccc6c(c5)sc5ccccc56)ccc5c4-c4ccccc4C5)n3)cc2)cc1 ; c1ccc(-c2nc(-c3cc4ccccc4c4ccccc34)nc(-c3cccc4oc5cc(-c6cccc7c6sc6ccccc67)ccc5c34)n2)cc1]

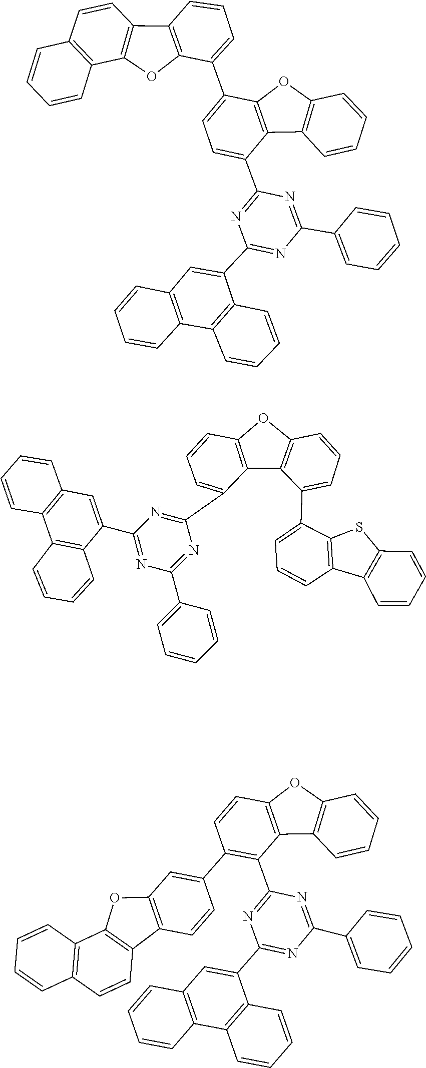 [SMILES: c1ccc(-c2nc(-c3cc4ccccc4c4ccccc34)nc(-c3ccc(-c4cccc5c4oc4c6ccccc6ccc54)c4oc5ccccc5c34)n2)cc1 ; c1ccc(-c2nc(-c3cc4ccccc4c4ccccc34)nc(-c3cccc4oc5cccc(-c6cccc7c6sc6ccccc67)c5c34)n2)cc1 ; c1ccc(-c2nc(-c3cc4ccccc4c4ccccc34)nc(-c3c(-c4ccc5c(c4)oc4c6ccccc6ccc54)ccc4oc5ccccc5c34)n2)cc1]

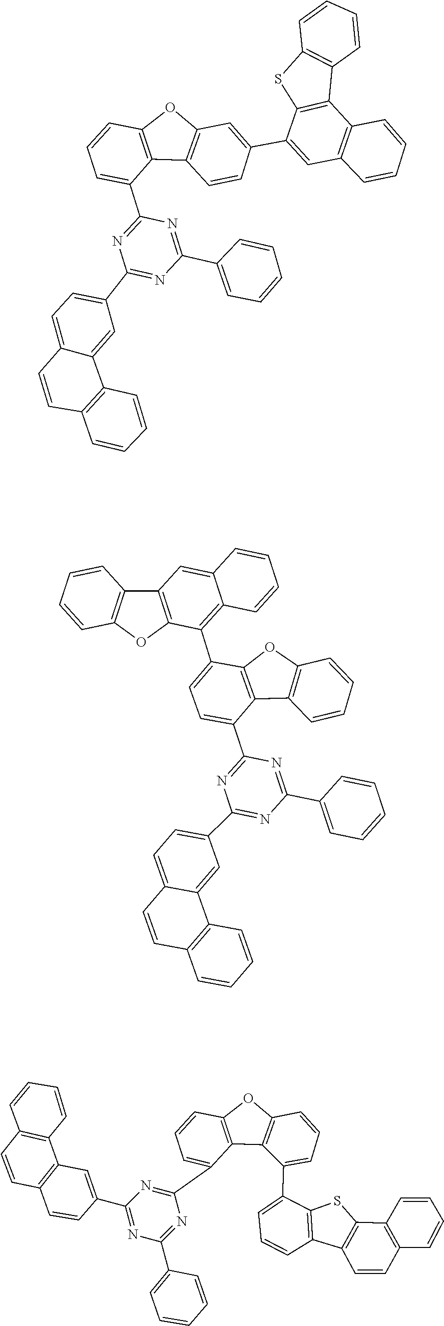 [SMILES: c1ccc(-c2nc(-c3ccc4ccc5ccccc5c4c3)nc(-c3cccc4oc5cc(-c6cc7ccccc7c7c6sc6ccccc67)ccc5c34)n2)cc1 ; c1ccc(-c2nc(-c3ccc4ccc5ccccc5c4c3)nc(-c3ccc(-c4c5ccccc5cc5c4oc4ccccc45)c4oc5ccccc5c34)n2)cc1 ; c1ccc(-c2nc(-c3ccc4ccc5ccccc5c4c3)nc(-c3cccc4oc5cccc(-c6cccc7c6sc6c8ccccc8ccc76)c5c34)n2)cc1]

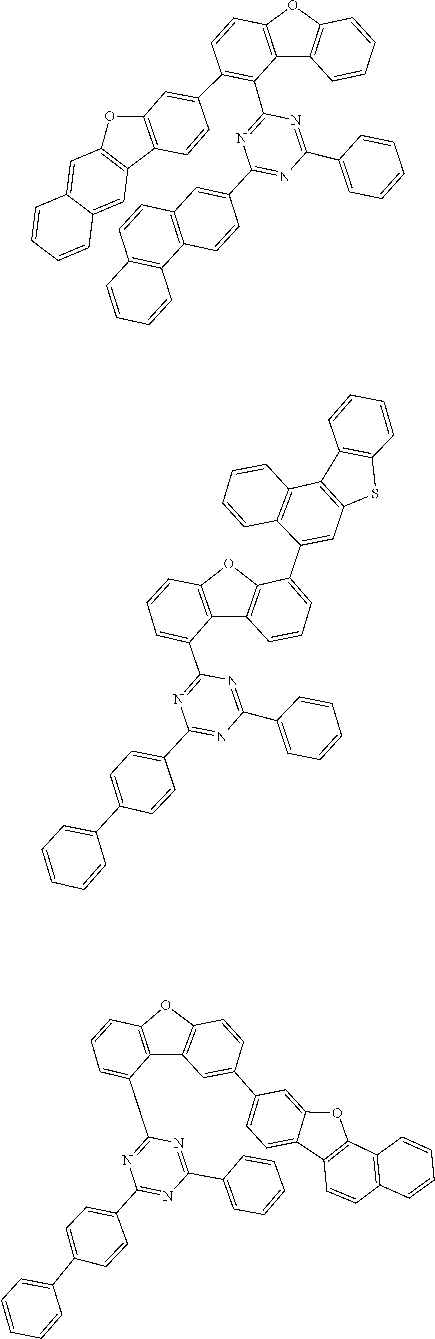 [SMILES: c1ccc(-c2nc(-c3ccc4c(ccc5ccccc54)c3)nc(-c3c(-c4ccc5c(c4)oc4cc6ccccc6cc45)ccc4oc5ccccc5c34)n2)cc1 ; c1ccc(-c2ccc(-c3nc(-c4ccccc4)nc(-c4cccc5oc6c(-c7cc8sc9ccccc9c8c8ccccc78)cccc6c45)n3)cc2)cc1 ; c1ccc(-c2ccc(-c3nc(-c4ccccc4)nc(-c4cccc5oc6ccc(-c7ccc8c(c7)oc7c9ccccc9ccc87)cc6c45)n3)cc2)cc1]

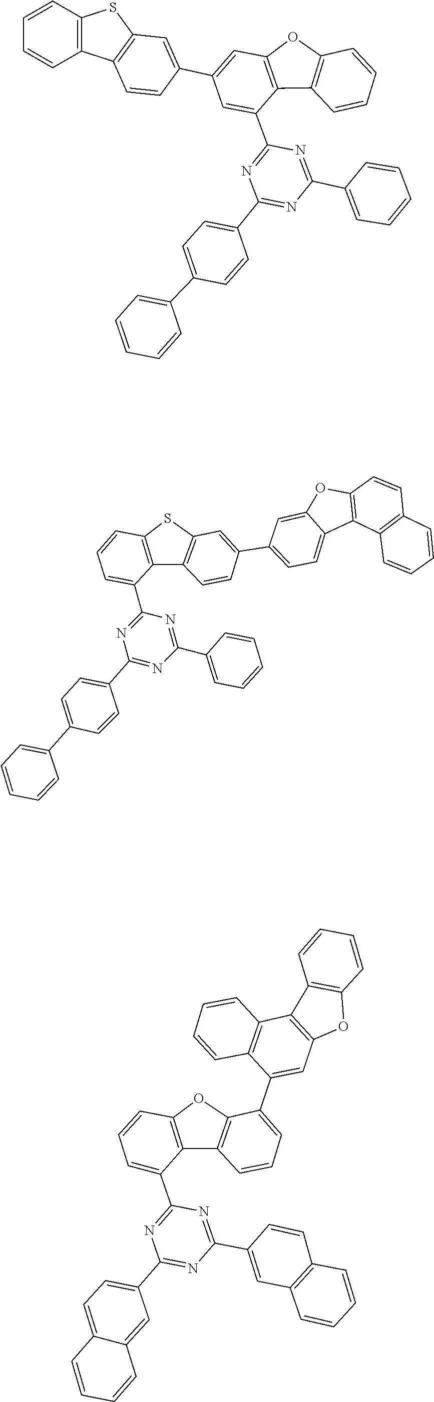 [SMILES: c1ccc(-c2ccc(-c3nc(-c4ccccc4)nc(-c4cc(-c5ccc6c(c5)sc5ccccc56)cc5oc6ccccc6c45)n3)cc2)cc1 ; c1ccc(-c2ccc(-c3nc(-c4ccccc4)nc(-c4cccc5sc6cc(-c7ccc8c(c7)oc7ccc9ccccc9c78)ccc6c45)n3)cc2)cc1 ; c1ccc2cc(-c3nc(-c4ccc5ccccc5c4)nc(-c4cccc5oc6c(-c7cc8oc9ccccc9c8c8ccccc78)cccc6c45)n3)ccc2c1]

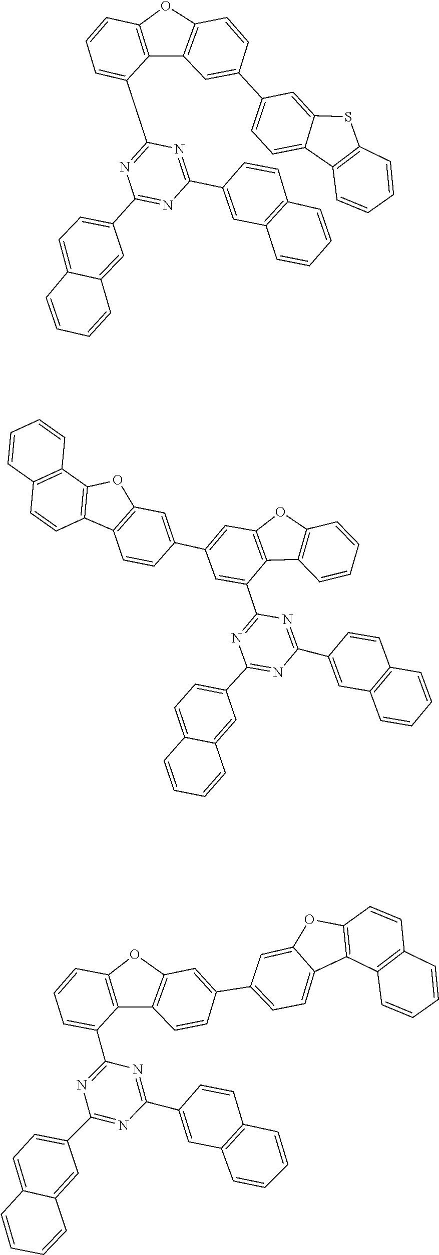 [SMILES: c1ccc2cc(-c3nc(-c4ccc5ccccc5c4)nc(-c4cccc5oc6ccc(-c7ccc8c(c7)sc7ccccc78)cc6c45)n3)ccc2c1 ; c1ccc2cc(-c3nc(-c4ccc5ccccc5c4)nc(-c4cc(-c5ccc6c(c5)oc5c7ccccc7ccc65)cc5oc6ccccc6c45)n3)ccc2c1 ; c1ccc2cc(-c3nc(-c4ccc5ccccc5c4)nc(-c4cccc5oc6cc(-c7ccc8c(c7)oc7ccc9ccccc9c78)ccc6c45)n3)ccc2c1]

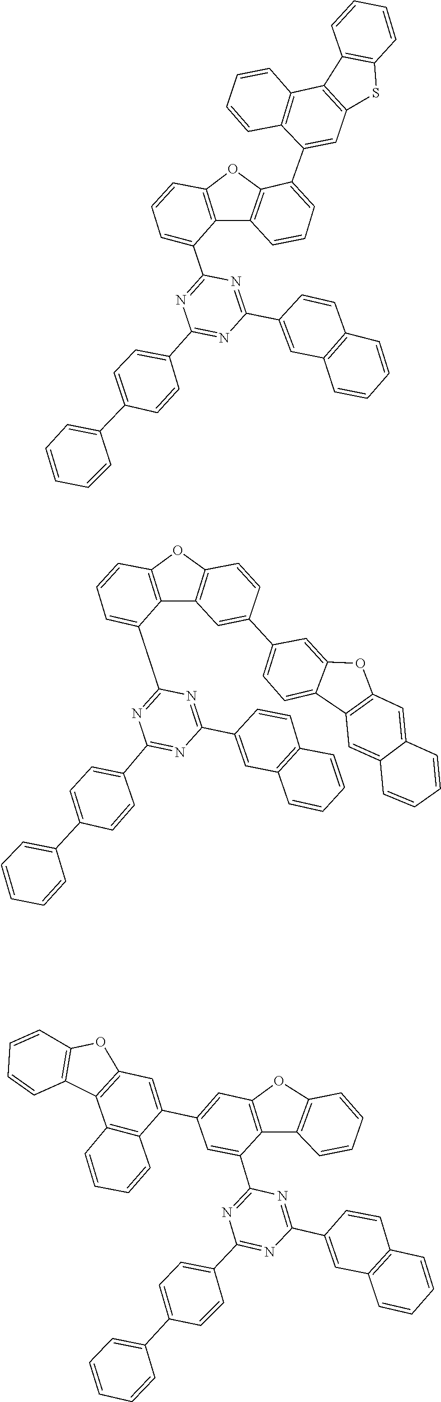 [SMILES: c1ccc(-c2ccc(-c3nc(-c4ccc5ccccc5c4)nc(-c4cccc5oc6c(-c7cc8sc9ccccc9c8c8ccccc78)cccc6c45)n3)cc2)cc1 ; c1ccc(-c2ccc(-c3nc(-c4ccc5ccccc5c4)nc(-c4cccc5oc6ccc(-c7ccc8c(c7)oc7cc9ccccc9cc78)cc6c45)n3)cc2)cc1 ; c1ccc(-c2ccc(-c3nc(-c4ccc5ccccc5c4)nc(-c4cc(-c5cc6oc7ccccc7c6c6ccccc56)cc5oc6ccccc6c45)n3)cc2)cc1]

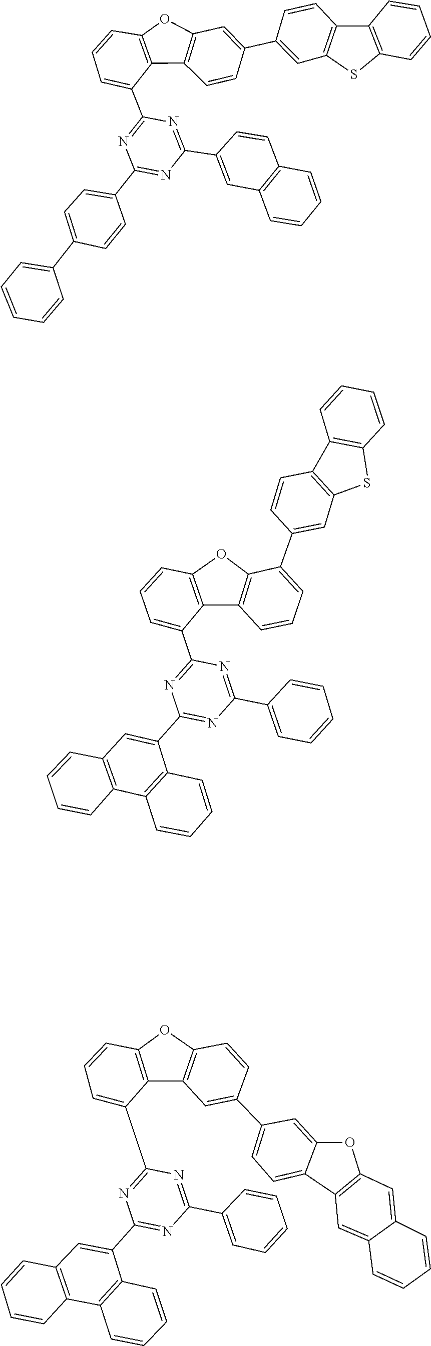 [SMILES: c1ccc(-c2ccc(-c3nc(-c4ccc5ccccc5c4)nc(-c4cccc5oc6cc(-c7ccc8c(c7)sc7ccccc78)ccc6c45)n3)cc2)cc1 ; c1ccc(-c2nc(-c3cc4ccccc4c4ccccc34)nc(-c3cccc4oc5c(-c6ccc7c(c6)sc6ccccc67)cccc5c34)n2)cc1 ; c1ccc(-c2nc(-c3cc4ccccc4c4ccccc34)nc(-c3cccc4oc5ccc(-c6ccc7c(c6)oc6cc8ccccc8cc67)cc5c34)n2)cc1]

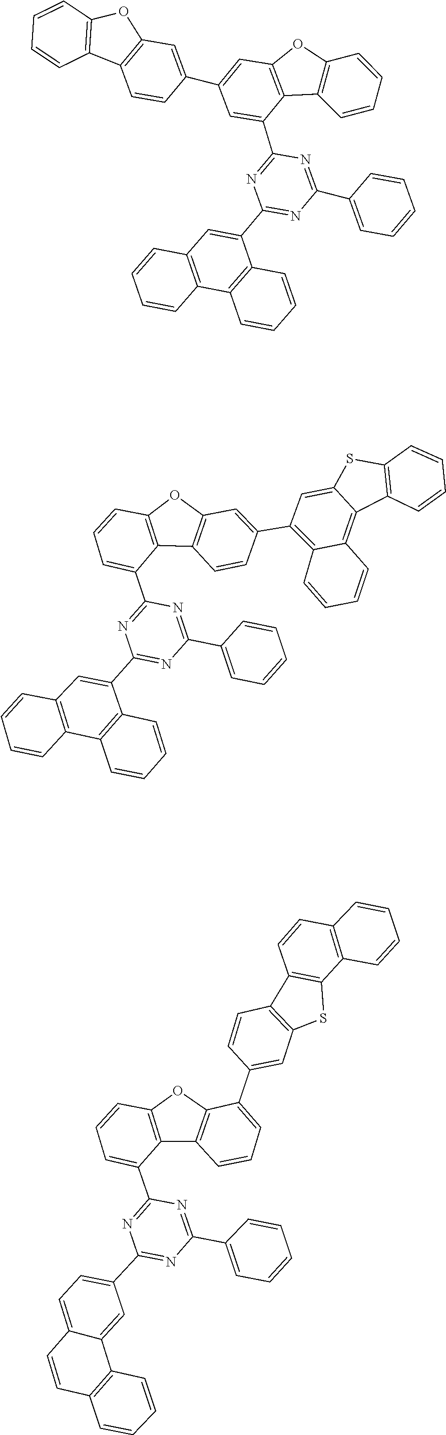 [SMILES: c1ccc(-c2nc(-c3cc4ccccc4c4ccccc34)nc(-c3cc(-c4ccc5c(c4)oc4ccccc45)cc4oc5ccccc5c34)n2)cc1 ; c1ccc(-c2nc(-c3cc4ccccc4c4ccccc34)nc(-c3cccc4oc5cc(-c6cc7sc8ccccc8c7c7ccccc67)ccc5c34)n2)cc1 ; c1ccc(-c2nc(-c3ccc4ccc5ccccc5c4c3)nc(-c3cccc4oc5c(-c6ccc7c(c6)sc6c8ccccc8ccc76)cccc5c34)n2)cc1]

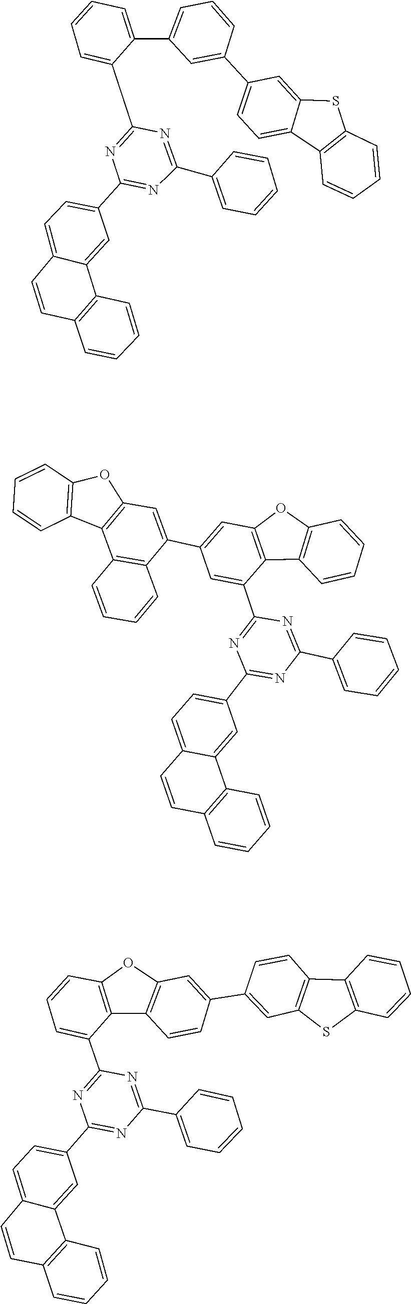 [SMILES: c1ccc(-c2nc(-c3ccc4ccc5ccccc5c4c3)nc(-c3ccccc3-c3cccc(-c4ccc5c(c4)sc4ccccc45)c3)n2)cc1 ; c1ccc(-c2nc(-c3ccc4ccc5ccccc5c4c3)nc(-c3cc(-c4cc5oc6ccccc6c5c5ccccc45)cc4oc5ccccc5c34)n2)cc1 ; c1ccc(-c2nc(-c3ccc4ccc5ccccc5c4c3)nc(-c3cccc4oc5cc(-c6ccc7c(c6)sc6ccccc67)ccc5c34)n2)cc1]

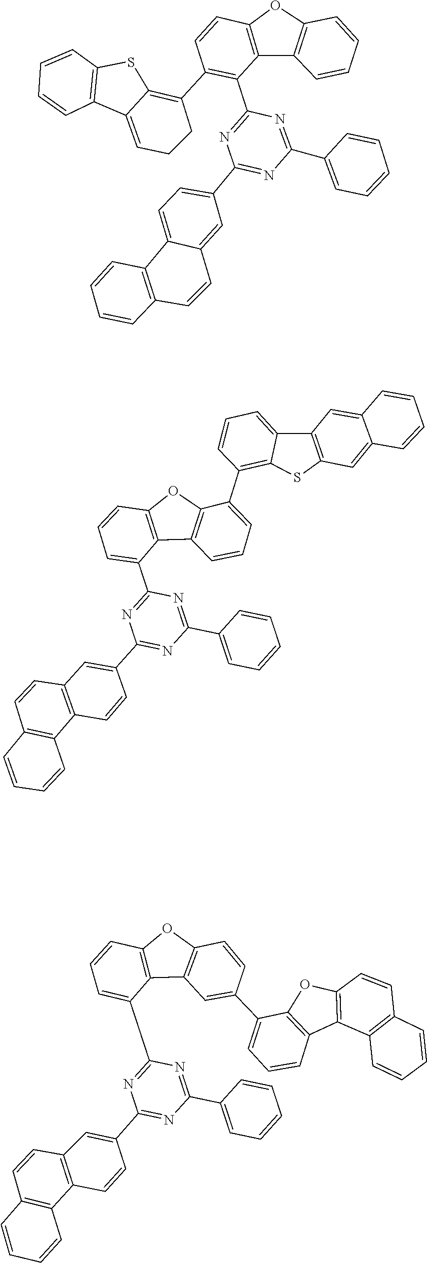 [SMILES: C1=c2c(sc3ccccc23)=C(c2ccc3oc4ccccc4c3c2-c2nc(-c3ccccc3)nc(-c3ccc4c(ccc5ccccc54)c3)n2)CC1 ; c1ccc(-c2nc(-c3ccc4c(ccc5ccccc54)c3)nc(-c3cccc4oc5c(-c6cccc7c6sc6cc8ccccc8cc67)cccc5c34)n2)cc1 ; c1ccc(-c2nc(-c3ccc4c(ccc5ccccc54)c3)nc(-c3cccc4oc5ccc(-c6cccc7c6oc6ccc8ccccc8c67)cc5c34)n2)cc1]

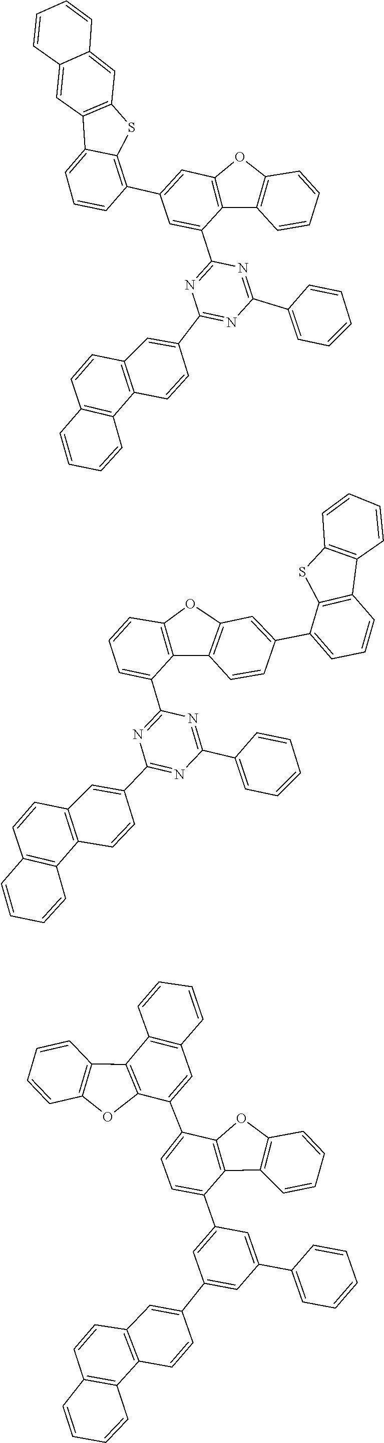 [SMILES: c1ccc(-c2nc(-c3ccc4c(ccc5ccccc54)c3)nc(-c3cc(-c4cccc5c4sc4cc6ccccc6cc45)cc4oc5ccccc5c34)n2)cc1 ; c1ccc(-c2nc(-c3ccc4c(ccc5ccccc54)c3)nc(-c3cccc4oc5cc(-c6cccc7c6sc6ccccc67)ccc5c34)n2)cc1 ; c1ccc(-c2cc(-c3ccc4c(ccc5ccccc54)c3)cc(-c3ccc(-c4cc5ccccc5c5c4oc4ccccc45)c4oc5ccccc5c34)c2)cc1]

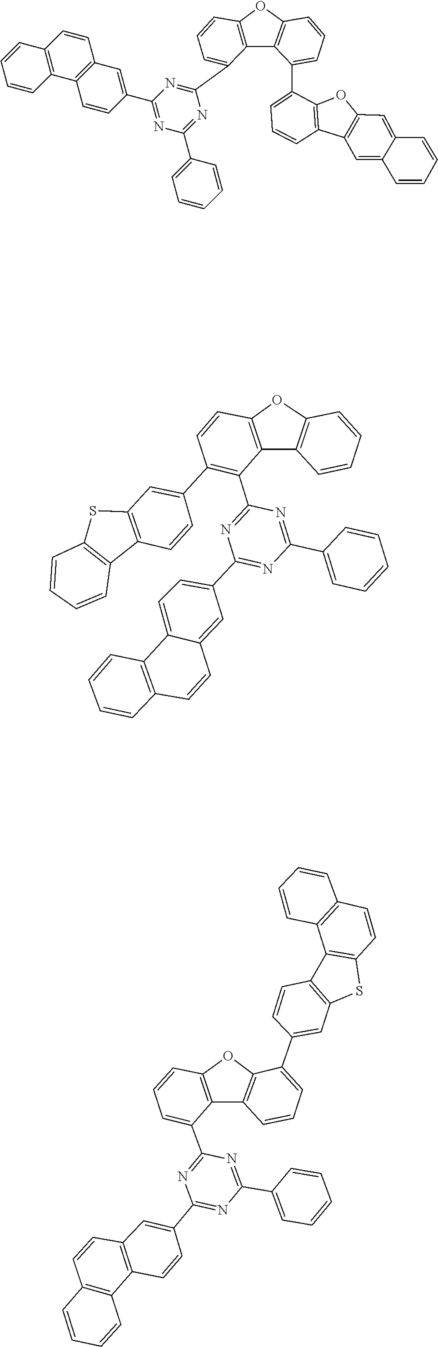 [SMILES: c1ccc(-c2nc(-c3ccc4c(ccc5ccccc54)c3)nc(-c3cccc4oc5cccc(-c6cccc7c6oc6cc8ccccc8cc67)c5c34)n2)cc1 ; c1ccc(-c2nc(-c3ccc4c(ccc5ccccc54)c3)nc(-c3c(-c4ccc5c(c4)sc4ccccc45)ccc4oc5ccccc5c34)n2)cc1 ; c1ccc(-c2nc(-c3ccc4c(ccc5ccccc54)c3)nc(-c3cccc4oc5c(-c6ccc7c(c6)sc6ccc8ccccc8c67)cccc5c34)n2)cc1]

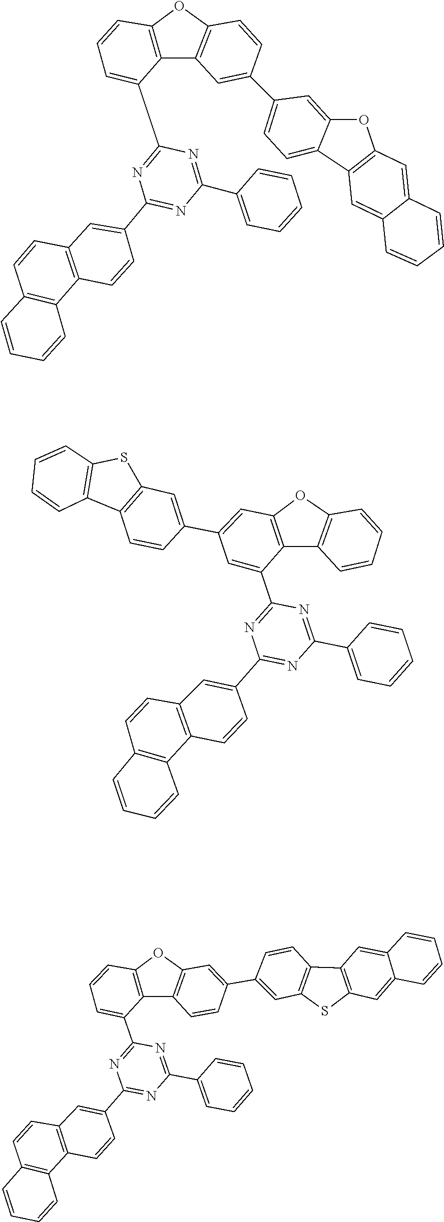 [SMILES: c1ccc(-c2nc(-c3ccc4c(ccc5ccccc54)c3)nc(-c3cccc4oc5ccc(-c6ccc7c(c6)oc6cc8ccccc8cc67)cc5c34)n2)cc1 ; c1ccc(-c2nc(-c3ccc4c(ccc5ccccc54)c3)nc(-c3cc(-c4ccc5c(c4)sc4ccccc45)cc4oc5ccccc5c34)n2)cc1 ; c1ccc(-c2nc(-c3ccc4c(ccc5ccccc54)c3)nc(-c3cccc4oc5cc(-c6ccc7c(c6)sc6cc8ccccc8cc67)ccc5c34)n2)cc1]

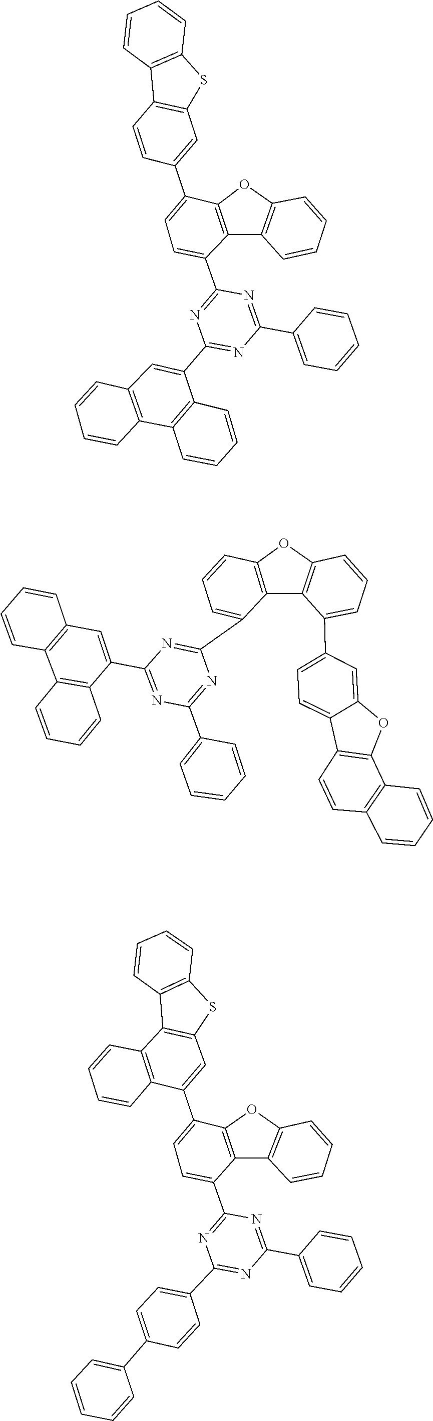 [SMILES: c1ccc(-c2nc(-c3cc4ccccc4c4ccccc34)nc(-c3ccc(-c4ccc5c(c4)sc4ccccc45)c4oc5ccccc5c34)n2)cc1 ; c1ccc(-c2nc(-c3cc4ccccc4c4ccccc34)nc(-c3cccc4oc5cccc(-c6ccc7c(c6)oc6c8ccccc8ccc76)c5c34)n2)cc1 ; c1ccc(-c2ccc(-c3nc(-c4ccccc4)nc(-c4ccc(-c5cc6sc7ccccc7c6c6ccccc56)c5oc6ccccc6c45)n3)cc2)cc1]

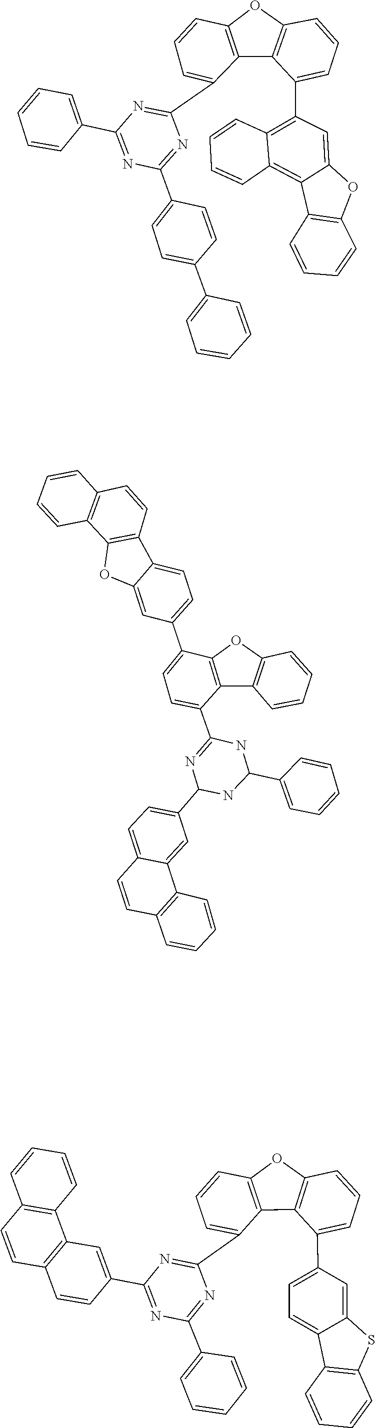

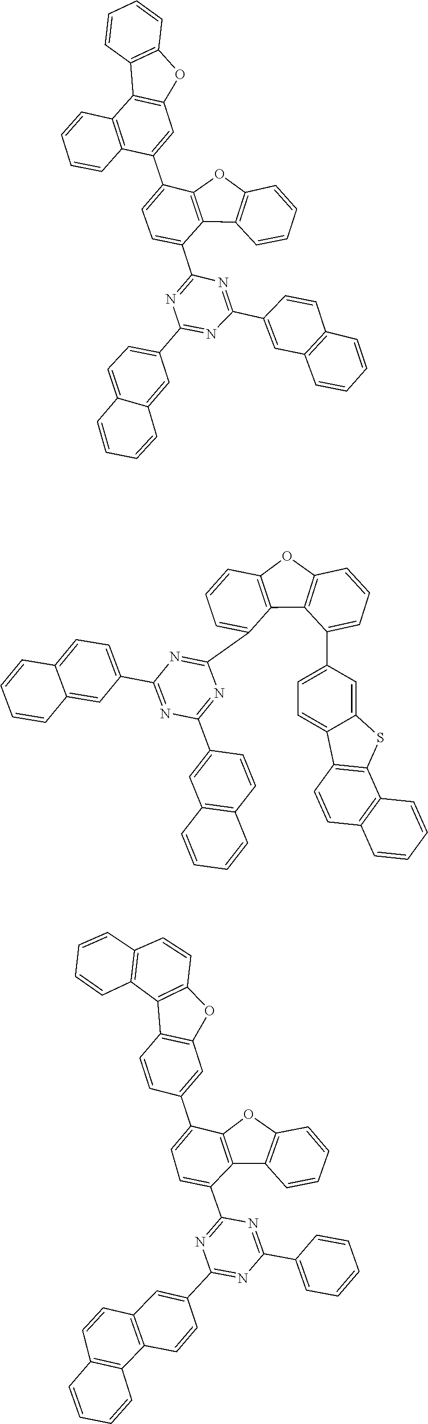 [SMILES: c1ccc2cc(-c3nc(-c4ccc5ccccc5c4)nc(-c4ccc(-c5cc6oc7ccccc7c6c6ccccc56)c5oc6ccccc6c45)n3)ccc2c1 ; c1ccc2cc(-c3nc(-c4ccc5ccccc5c4)nc(-c4cccc5oc6cccc(-c7ccc8c(c7)sc7c9ccccc9ccc87)c6c45)n3)ccc2c1 ; c1ccc(-c2nc(-c3ccc4c(ccc5ccccc54)c3)nc(-c3ccc(-c4ccc5c(c4)oc4ccc6ccccc6c45)c4oc5ccccc5c34)n2)cc1]

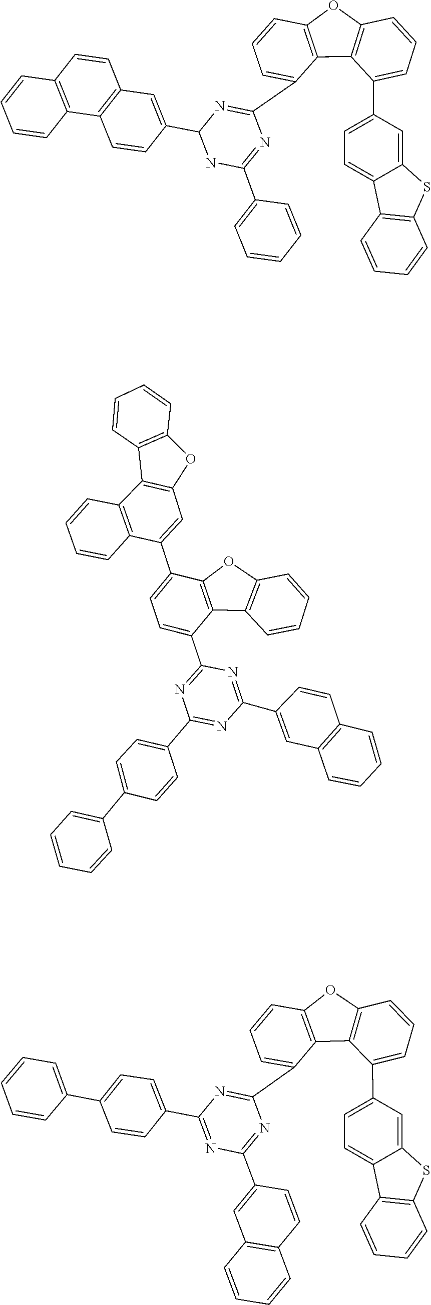

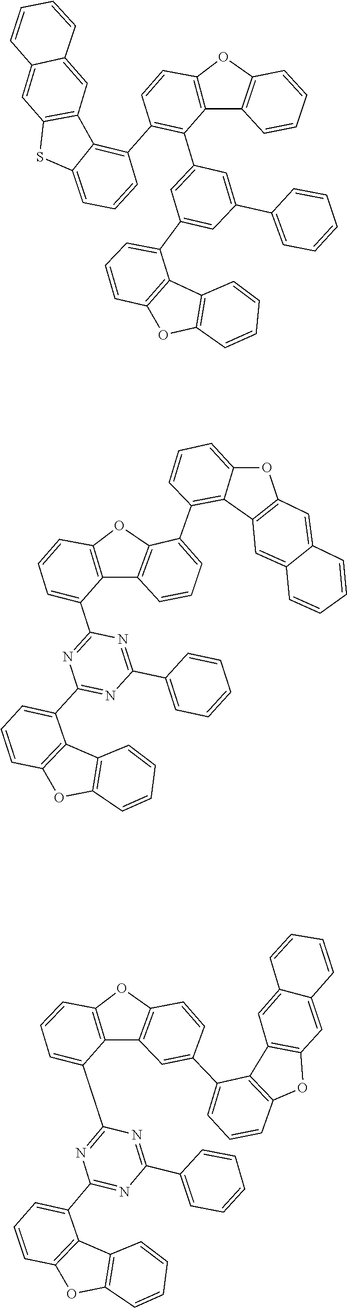 [SMILES: c1ccc(-c2cc(-c3cccc4oc5ccccc5c34)cc(-c3c(-c4cccc5sc6cc7ccccc7cc6c45)ccc4oc5ccccc5c34)c2)cc1 ; c1ccc(-c2nc(-c3cccc4oc5ccccc5c34)nc(-c3cccc4oc5c(-c6cccc7oc8cc9ccccc9cc8c67)cccc5c34)n2)cc1 ; c1ccc(-c2nc(-c3cccc4oc5ccccc5c34)nc(-c3cccc4oc5ccc(-c6cccc7oc8cc9ccccc9cc8c67)cc5c34)n2)cc1]

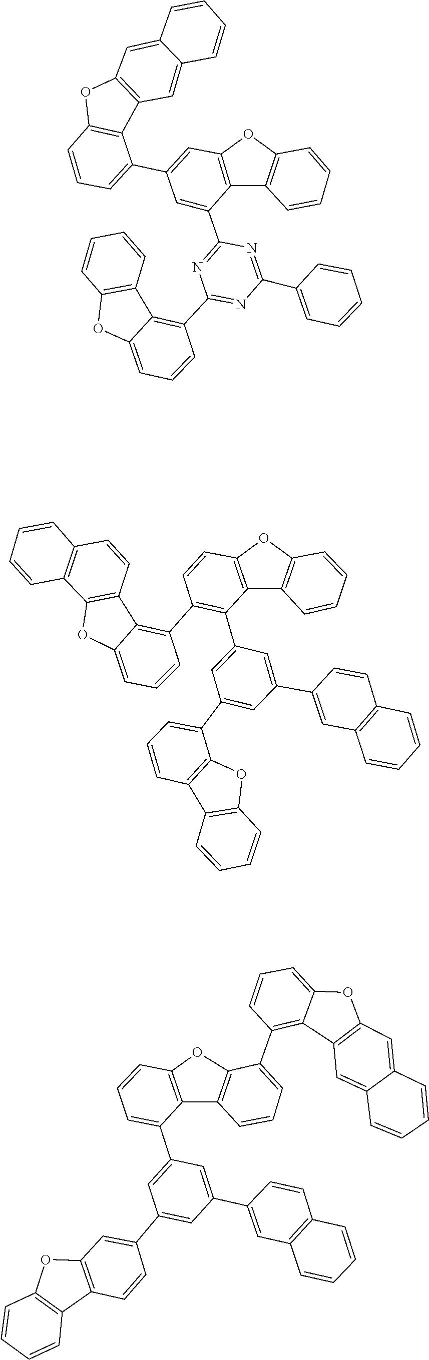 [SMILES: c1ccc(-c2nc(-c3cccc4oc5ccccc5c34)nc(-c3cc(-c4cccc5oc6cc7ccccc7cc6c45)cc4oc5ccccc5c34)n2)cc1 ; c1ccc2cc(-c3cc(-c4cccc5c4oc4ccccc45)cc(-c4c(-c5cccc6oc7c8ccccc8ccc7c56)ccc5oc6ccccc6c45)c3)ccc2c1 ; c1ccc2cc(-c3cc(-c4ccc5c(c4)oc4ccccc45)cc(-c4cccc5oc6c(-c7cccc8oc9cc%10ccccc%10cc9c78)cccc6c45)c3)ccc2c1]

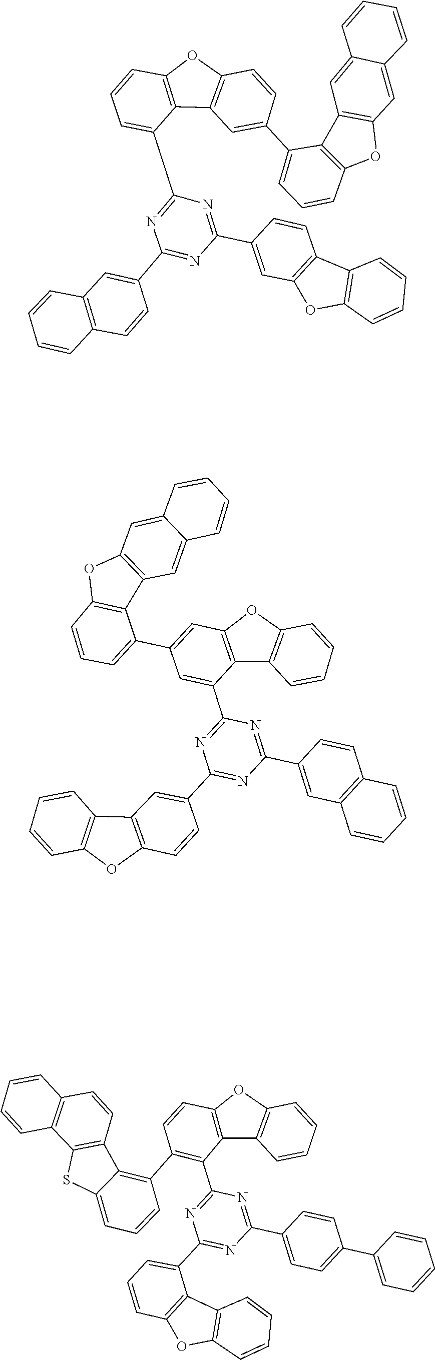 [SMILES: c1ccc2cc(-c3nc(-c4ccc5c(c4)oc4ccccc45)nc(-c4cccc5oc6ccc(-c7cccc8oc9cc%10ccccc%10cc9c78)cc6c45)n3)ccc2c1 ; c1ccc2cc(-c3nc(-c4ccc5oc6ccccc6c5c4)nc(-c4cc(-c5cccc6oc7cc8ccccc8cc7c56)cc5oc6ccccc6c45)n3)ccc2c1 ; c1ccc(-c2ccc(-c3nc(-c4cccc5oc6ccccc6c45)nc(-c4c(-c5cccc6sc7c8ccccc8ccc7c56)ccc5oc6ccccc6c45)n3)cc2)cc1]

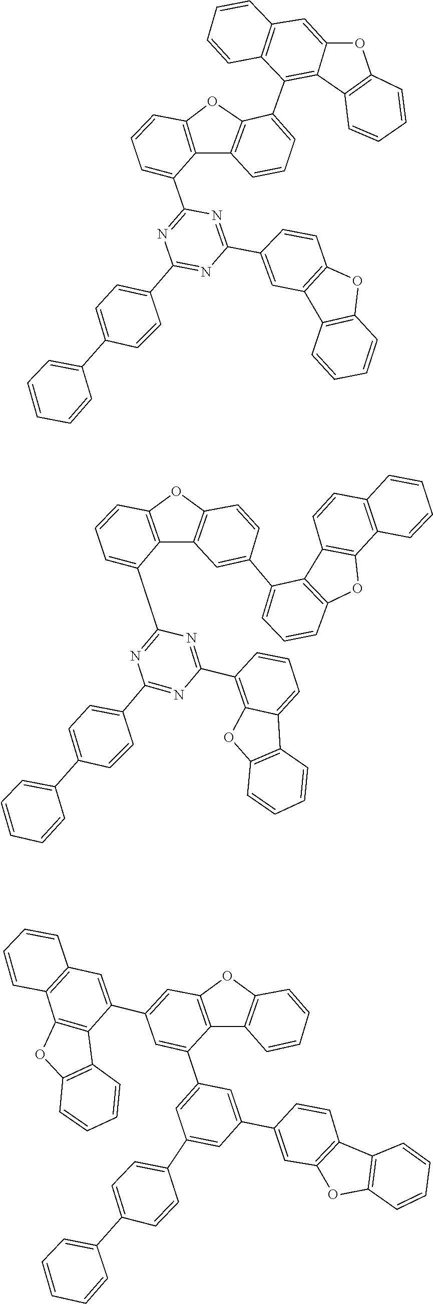 [SMILES: c1ccc(-c2ccc(-c3nc(-c4ccc5oc6ccccc6c5c4)nc(-c4cccc5oc6c(-c7c8ccccc8cc8oc9ccccc9c78)cccc6c45)n3)cc2)cc1 ; c1ccc(-c2ccc(-c3nc(-c4cccc5c4oc4ccccc45)nc(-c4cccc5oc6ccc(-c7cccc8oc9c%10ccccc%10ccc9c78)cc6c45)n3)cc2)cc1 ; c1ccc(-c2ccc(-c3cc(-c4ccc5c(c4)oc4ccccc45)cc(-c4cc(-c5cc6ccccc6c6oc7ccccc7c56)cc5oc6ccccc6c45)c3)cc2)cc1]

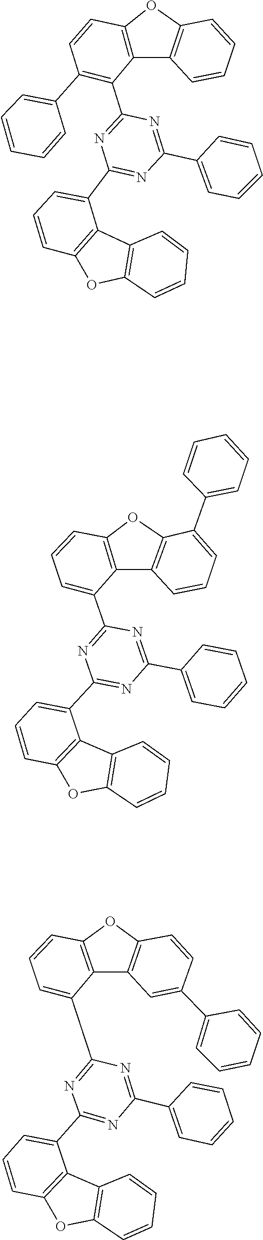 [SMILES: c1ccc(-c2nc(-c3cccc4oc5ccccc5c34)nc(-c3c(-c4ccccc4)ccc4oc5ccccc5c34)n2)cc1 ; c1ccc(-c2nc(-c3cccc4oc5ccccc5c34)nc(-c3cccc4oc5c(-c6ccccc6)cccc5c34)n2)cc1 ; c1ccc(-c2ccc3oc4cccc(-c5nc(-c6ccccc6)nc(-c6cccc7oc8ccccc8c67)n5)c4c3c2)cc1]

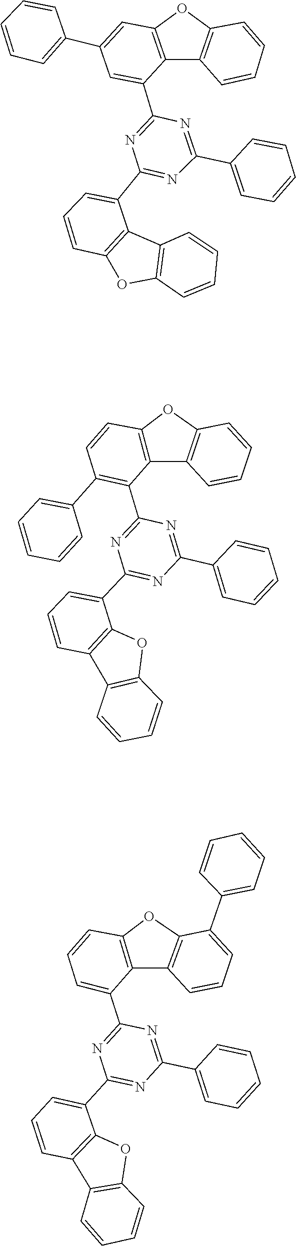 [SMILES: c1ccc(-c2cc(-c3nc(-c4ccccc4)nc(-c4cccc5oc6ccccc6c45)n3)c3c(c2)oc2ccccc23)cc1 ; c1ccc(-c2nc(-c3cccc4c3oc3ccccc34)nc(-c3c(-c4ccccc4)ccc4oc5ccccc5c34)n2)cc1 ; c1ccc(-c2nc(-c3cccc4c3oc3ccccc34)nc(-c3cccc4oc5c(-c6ccccc6)cccc5c34)n2)cc1]

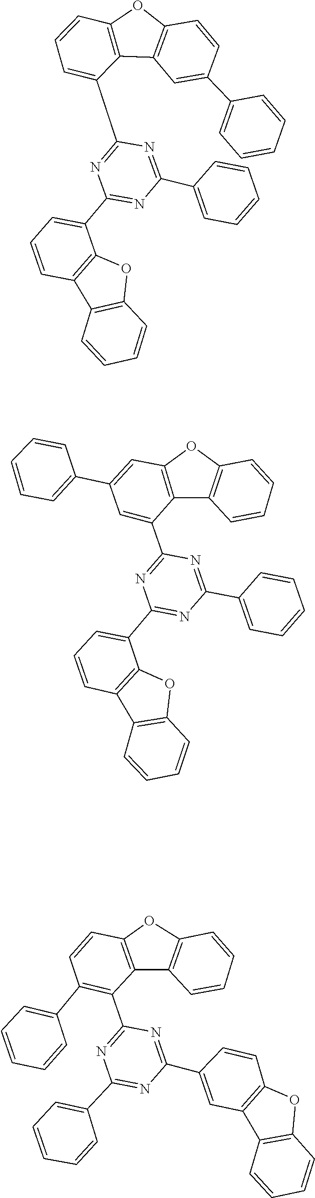 [SMILES: c1ccc(-c2ccc3oc4cccc(-c5nc(-c6ccccc6)nc(-c6cccc7c6oc6ccccc67)n5)c4c3c2)cc1 ; c1ccc(-c2cc(-c3nc(-c4ccccc4)nc(-c4cccc5c4oc4ccccc45)n3)c3c(c2)oc2ccccc23)cc1 ; c1ccc(-c2nc(-c3ccc4oc5ccccc5c4c3)nc(-c3c(-c4ccccc4)ccc4oc5ccccc5c34)n2)cc1]

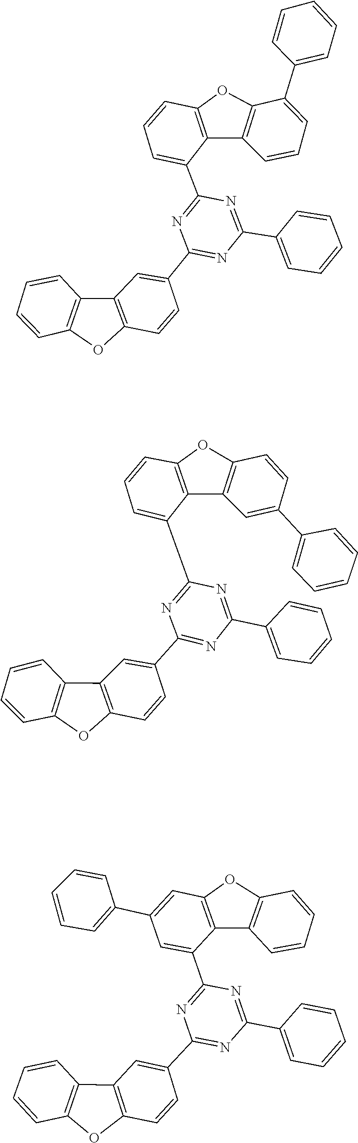

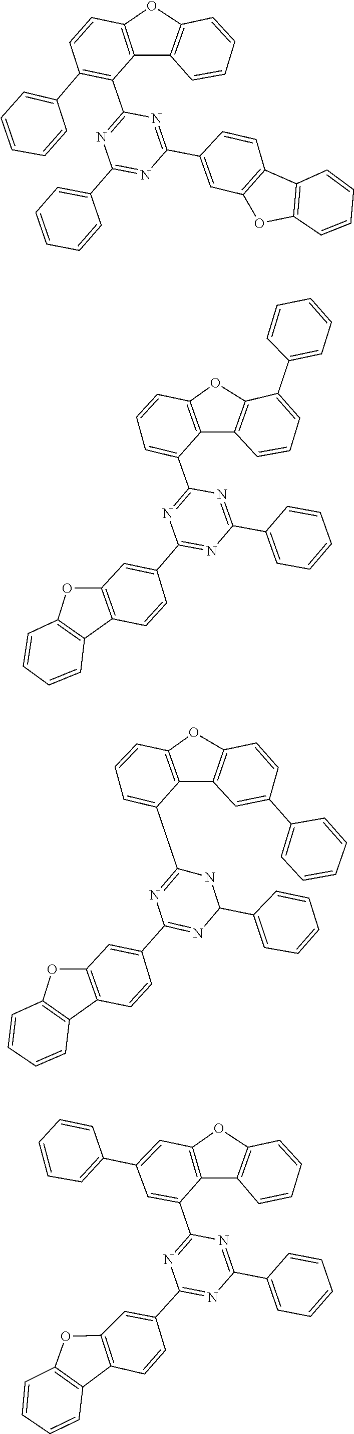

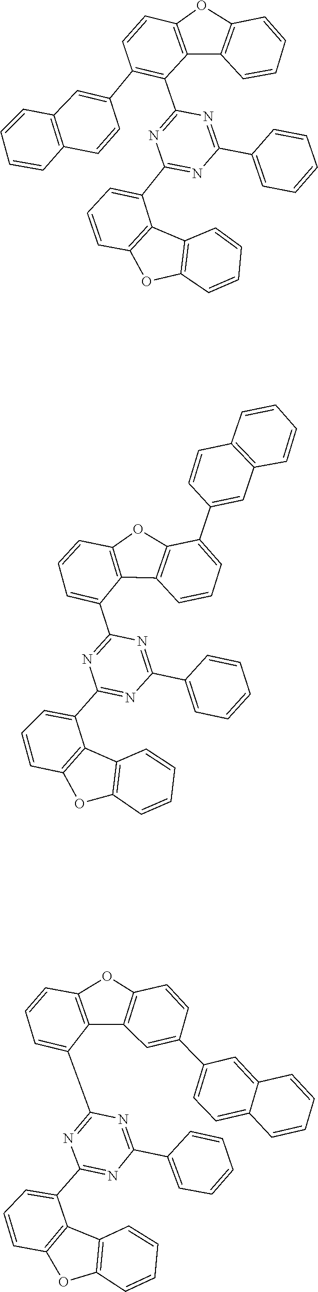 [SMILES: c1ccc(-c2nc(-c3cccc4oc5ccccc5c34)nc(-c3c(-c4ccc5ccccc5c4)ccc4oc5ccccc5c34)n2)cc1 ; c1ccc(-c2nc(-c3cccc4oc5ccccc5c34)nc(-c3cccc4oc5c(-c6ccc7ccccc7c6)cccc5c34)n2)cc1 ; c1ccc(-c2nc(-c3cccc4oc5ccccc5c34)nc(-c3cccc4oc5ccc(-c6ccc7ccccc7c6)cc5c34)n2)cc1]

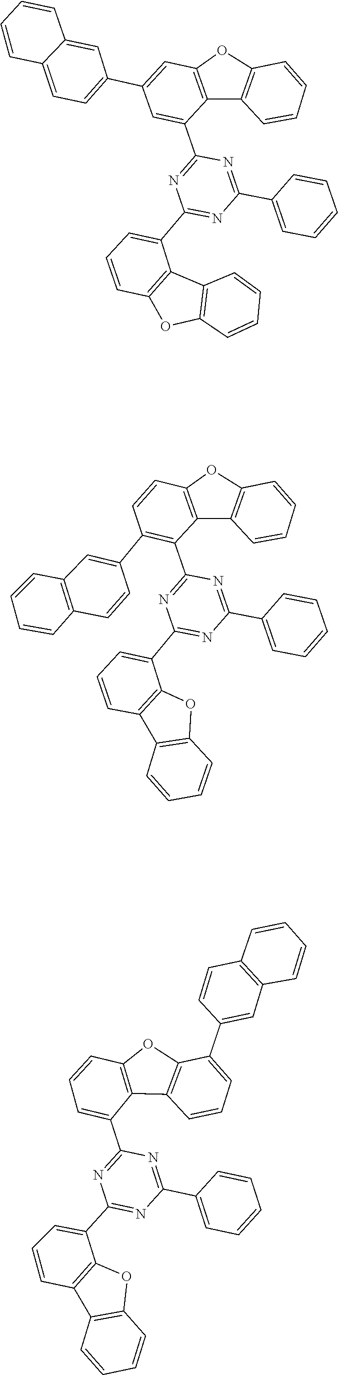 [SMILES: c1ccc(-c2nc(-c3cccc4oc5ccccc5c34)nc(-c3cc(-c4ccc5ccccc5c4)cc4oc5ccccc5c34)n2)cc1 ; c1ccc(-c2nc(-c3cccc4c3oc3ccccc34)nc(-c3c(-c4ccc5ccccc5c4)ccc4oc5ccccc5c34)n2)cc1 ; c1ccc(-c2nc(-c3cccc4c3oc3ccccc34)nc(-c3cccc4oc5c(-c6ccc7ccccc7c6)cccc5c34)n2)cc1]

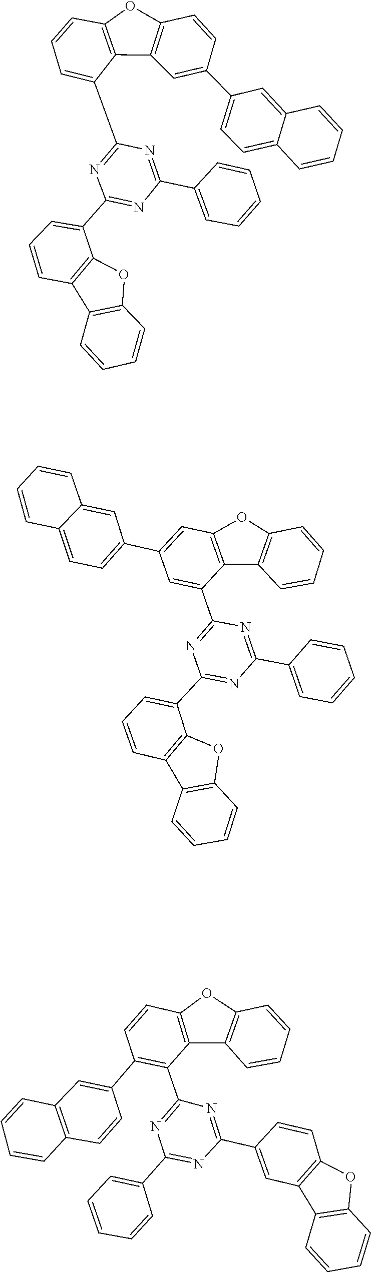 [SMILES: c1ccc(-c2nc(-c3cccc4c3oc3ccccc34)nc(-c3cccc4oc5ccc(-c6ccc7ccccc7c6)cc5c34)n2)cc1 ; c1ccc(-c2nc(-c3cccc4c3oc3ccccc34)nc(-c3cc(-c4ccc5ccccc5c4)cc4oc5ccccc5c34)n2)cc1 ; c1ccc(-c2nc(-c3ccc4oc5ccccc5c4c3)nc(-c3c(-c4ccc5ccccc5c4)ccc4oc5ccccc5c34)n2)cc1]

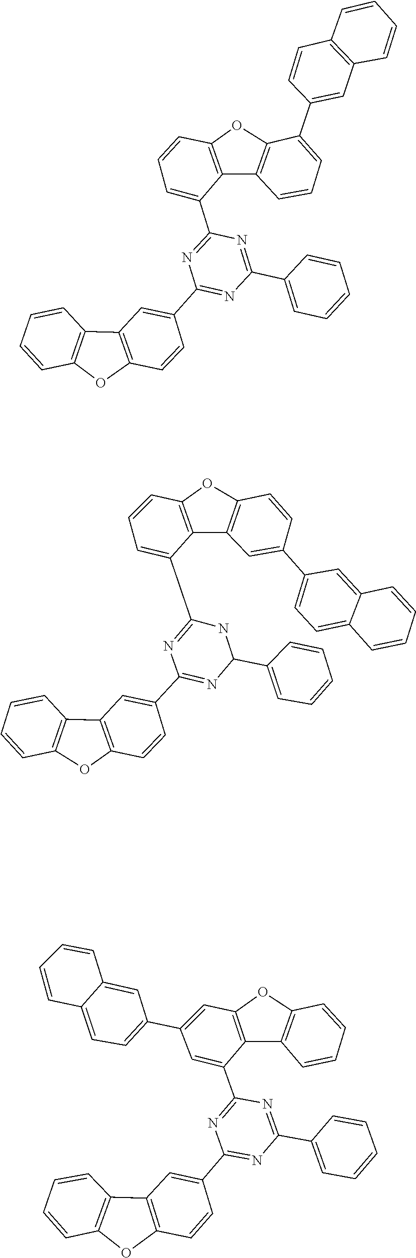

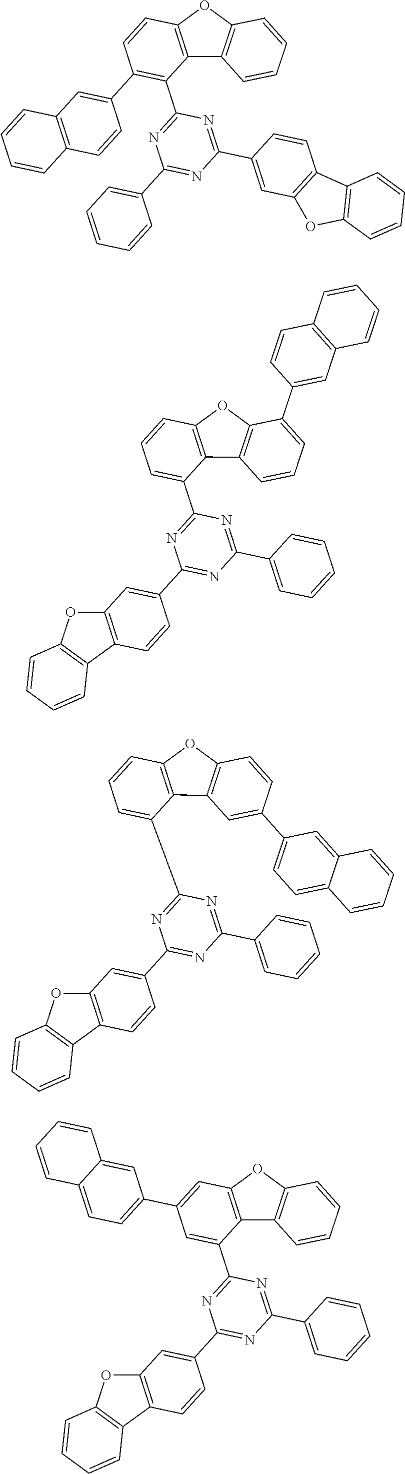

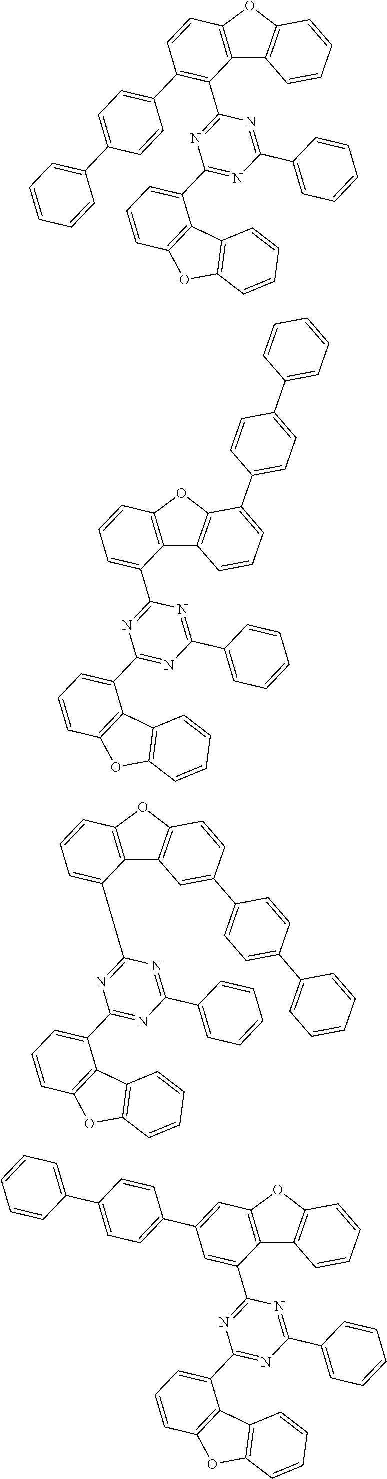

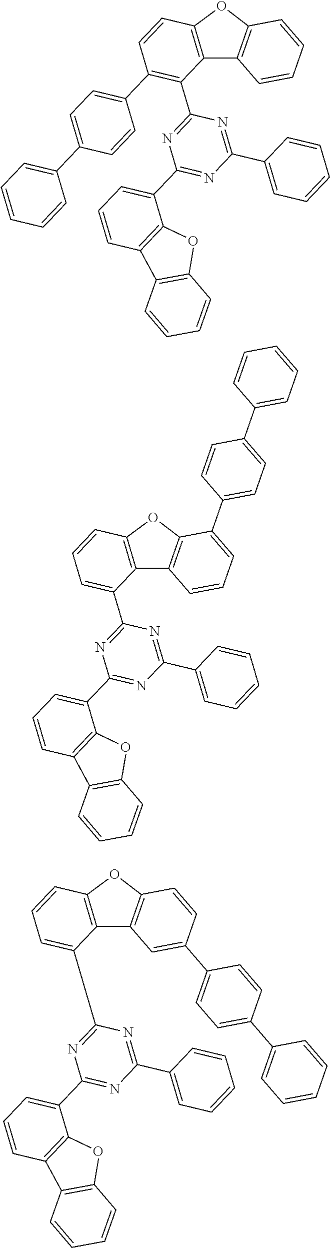

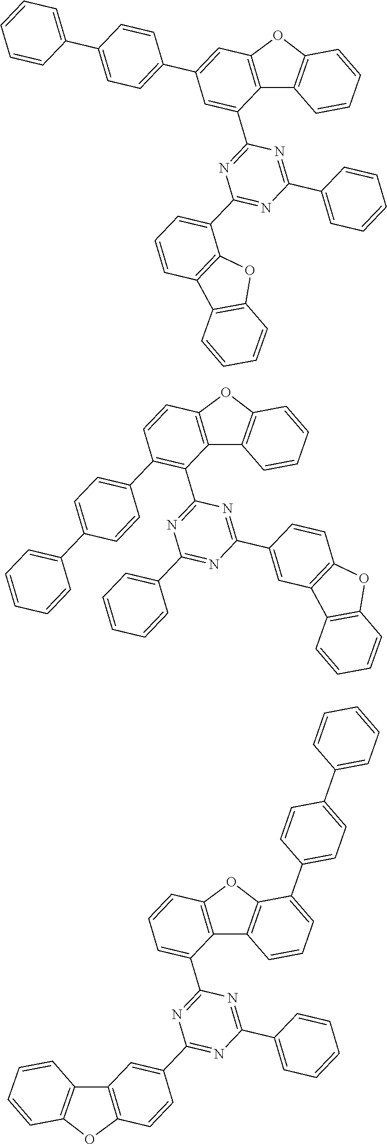

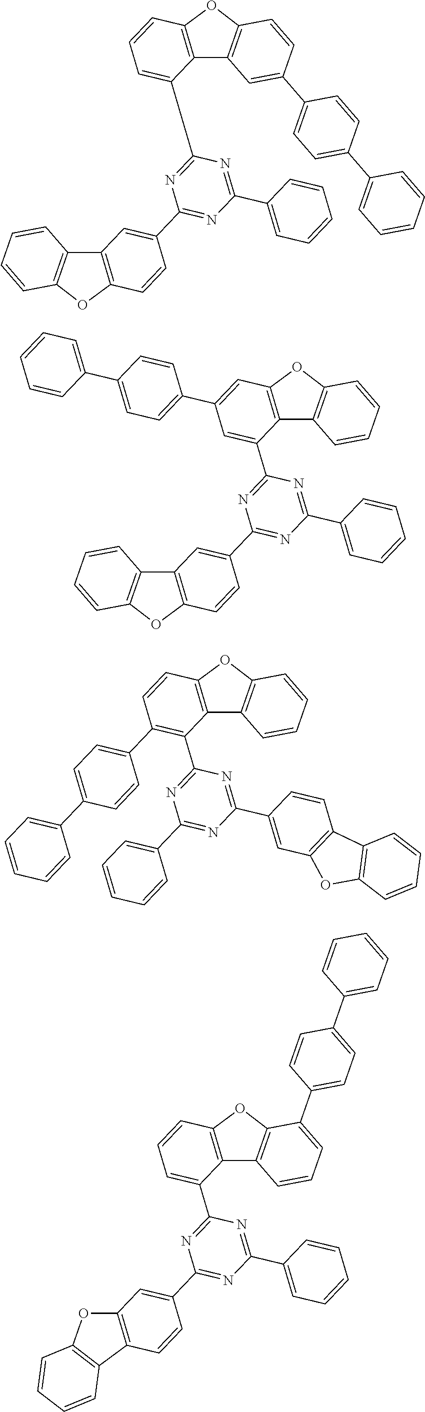

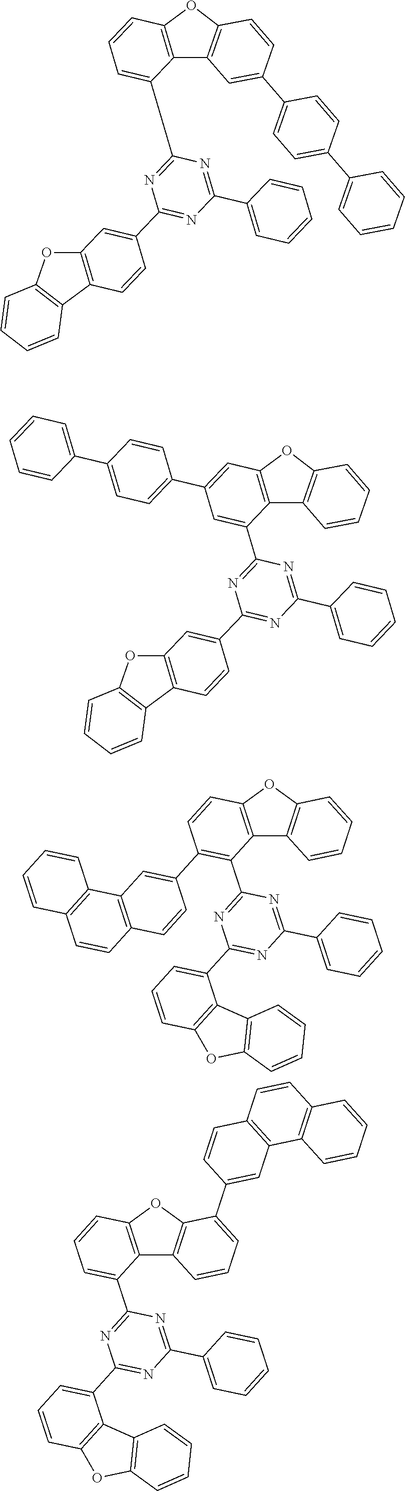

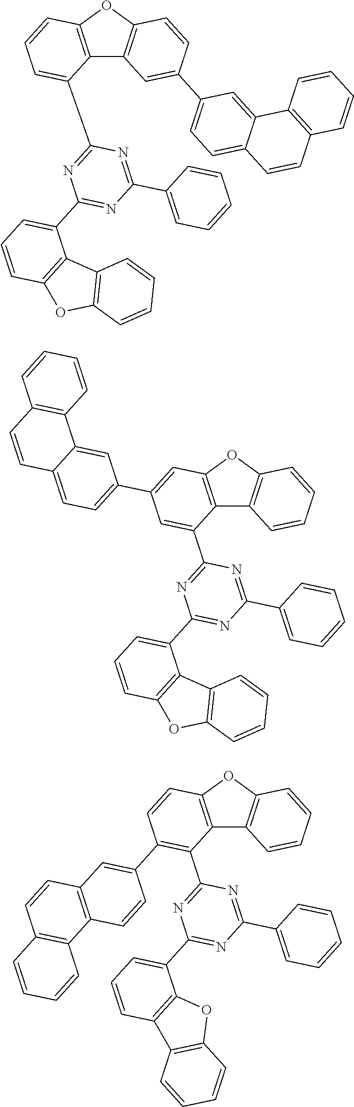

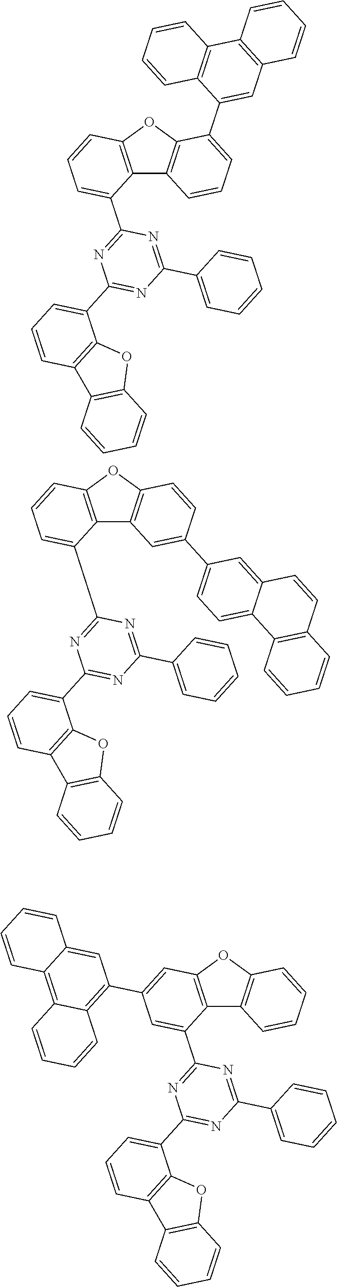

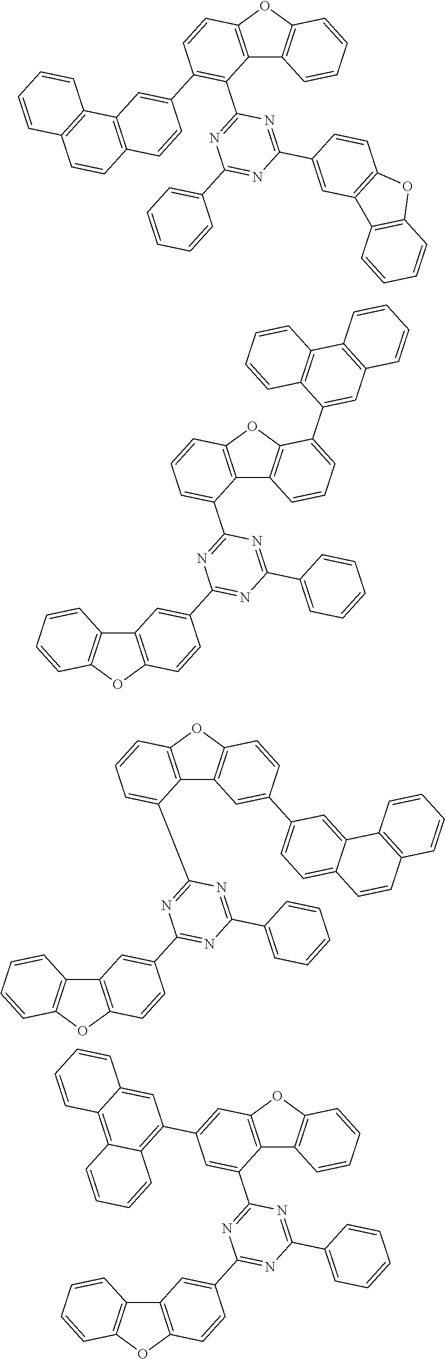

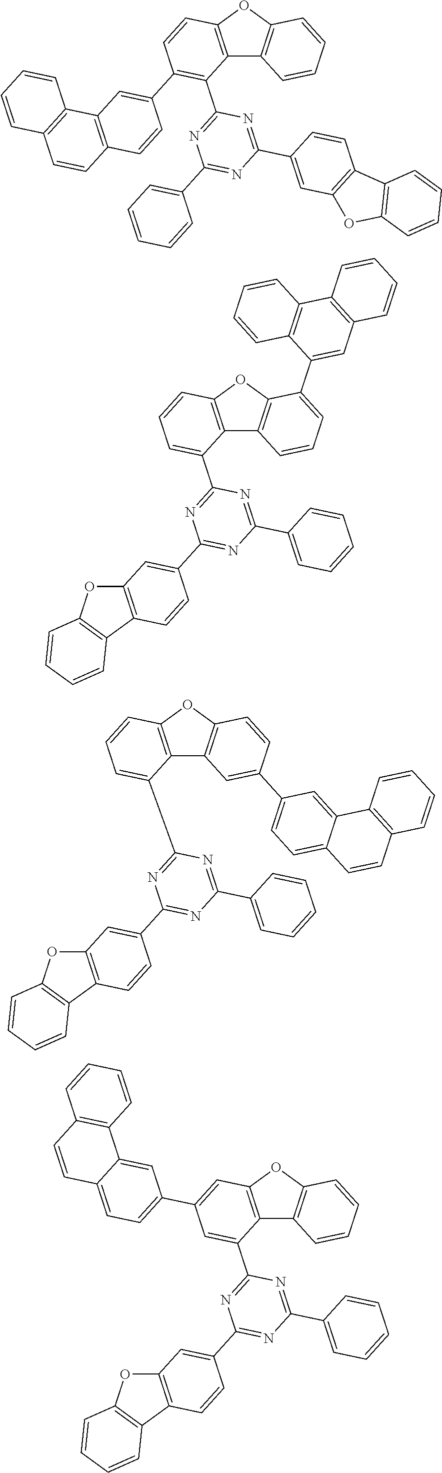

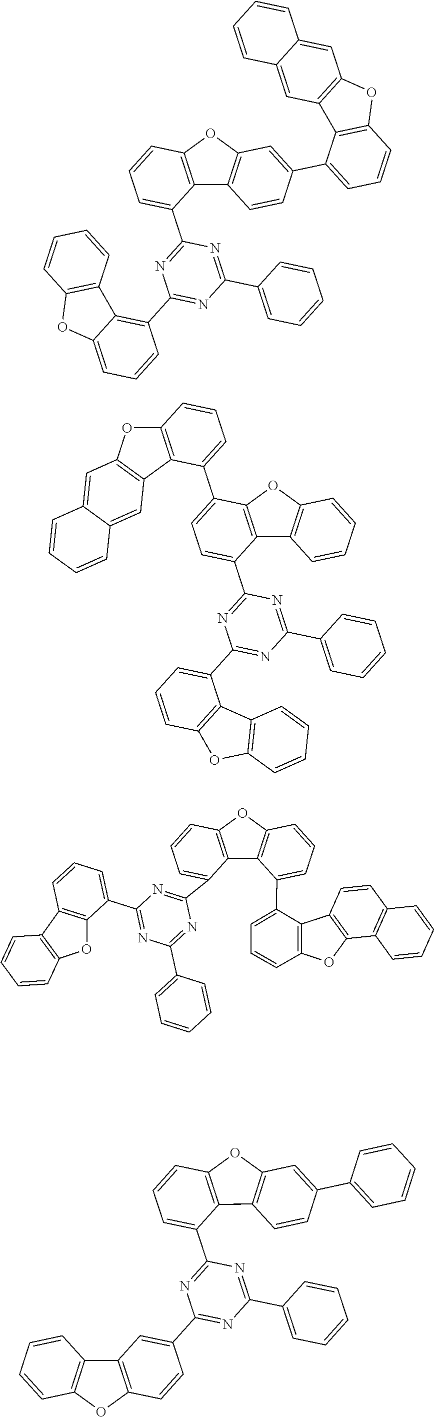 [SMILES: c1ccc(-c2nc(-c3cccc4oc5ccccc5c34)nc(-c3cccc4oc5cc(-c6cccc7oc8cc9ccccc9cc8c67)ccc5c34)n2)cc1 ; c1ccc(-c2nc(-c3cccc4oc5ccccc5c34)nc(-c3ccc(-c4cccc5oc6cc7ccccc7cc6c45)c4oc5ccccc5c34)n2)cc1 ; c1ccc(-c2nc(-c3cccc4c3oc3ccccc34)nc(-c3cccc4oc5cccc(-c6cccc7oc8c9ccccc9ccc8c67)c5c34)n2)cc1 ; c1ccc(-c2ccc3c(c2)oc2cccc(-c4nc(-c5ccccc5)nc(-c5ccc6oc7ccccc7c6c5)n4)c23)cc1]

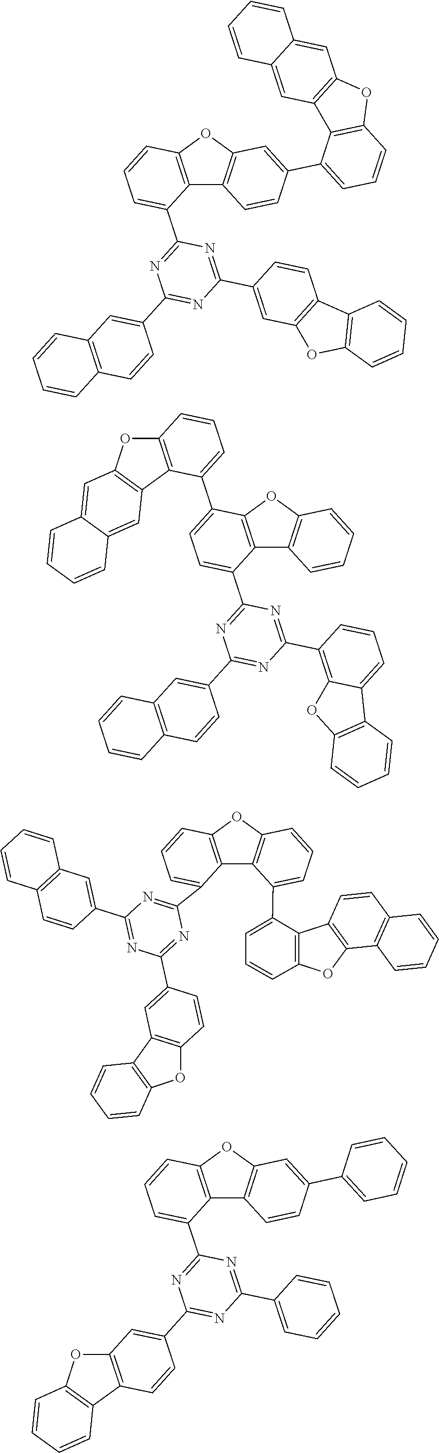 [SMILES: c1ccc2cc(-c3nc(-c4ccc5c(c4)oc4ccccc45)nc(-c4cccc5oc6cc(-c7cccc8oc9cc%10ccccc%10cc9c78)ccc6c45)n3)ccc2c1 ; c1ccc2cc(-c3nc(-c4cccc5c4oc4ccccc45)nc(-c4ccc(-c5cccc6oc7cc8ccccc8cc7c56)c5oc6ccccc6c45)n3)ccc2c1 ; c1ccc2cc(-c3nc(-c4ccc5oc6ccccc6c5c4)nc(-c4cccc5oc6cccc(-c7cccc8oc9c%10ccccc%10ccc9c78)c6c45)n3)ccc2c1 ; c1ccc(-c2ccc3c(c2)oc2cccc(-c4nc(-c5ccccc5)nc(-c5ccc6c(c5)oc5ccccc56)n4)c23)cc1]

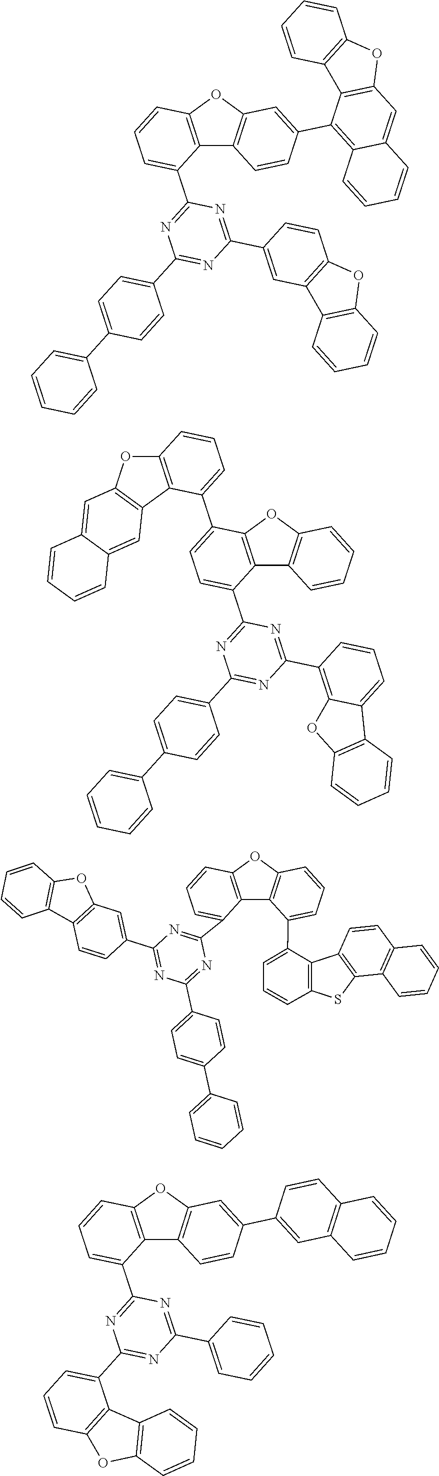 [SMILES: c1ccc(-c2ccc(-c3nc(-c4ccc5oc6ccccc6c5c4)nc(-c4cccc5oc6cc(-c7c8ccccc8cc8oc9ccccc9c78)ccc6c45)n3)cc2)cc1 ; c1ccc(-c2ccc(-c3nc(-c4cccc5c4oc4ccccc45)nc(-c4ccc(-c5cccc6oc7cc8ccccc8cc7c56)c5oc6ccccc6c45)n3)cc2)cc1 ; c1ccc(-c2ccc(-c3nc(-c4ccc5c(c4)oc4ccccc45)nc(-c4cccc5oc6cccc(-c7cccc8sc9c%10ccccc%10ccc9c78)c6c45)n3)cc2)cc1 ; c1ccc(-c2nc(-c3cccc4oc5ccccc5c34)nc(-c3cccc4oc5cc(-c6ccc7ccccc7c6)ccc5c34)n2)cc1]

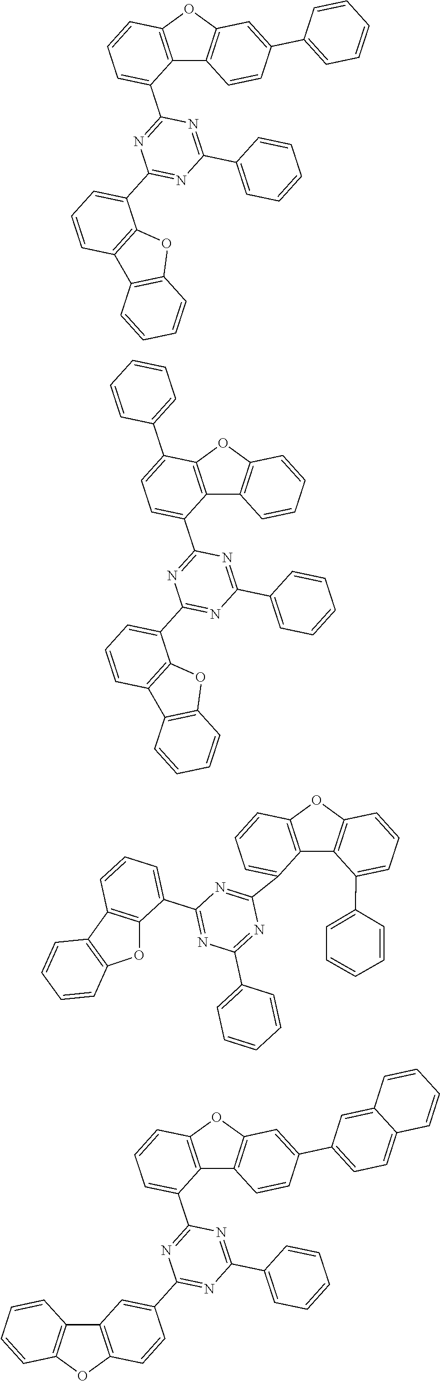

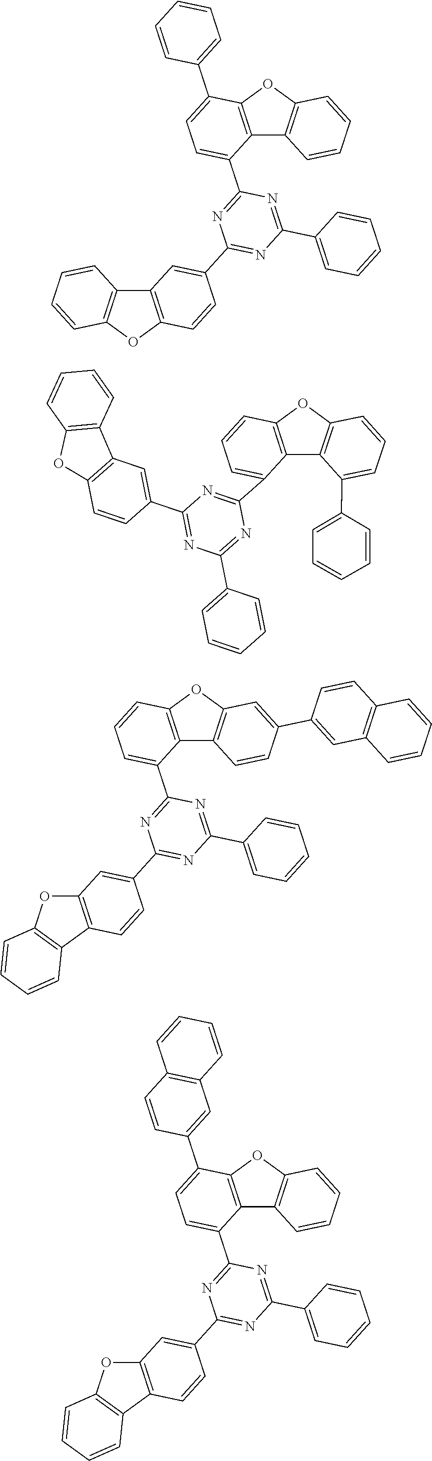

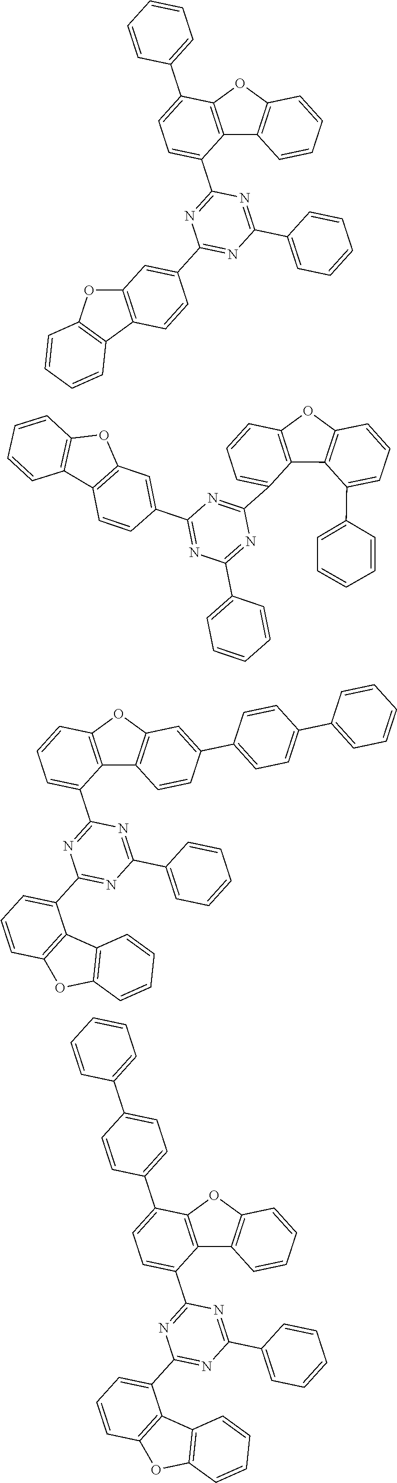

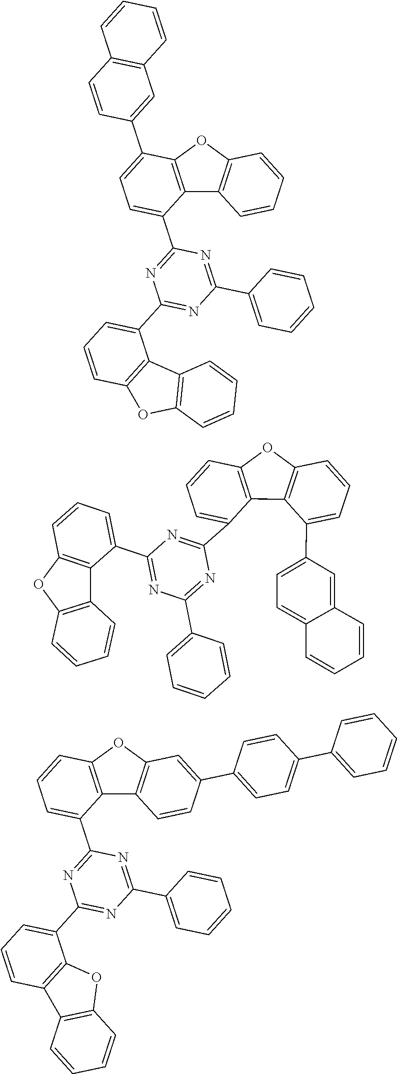

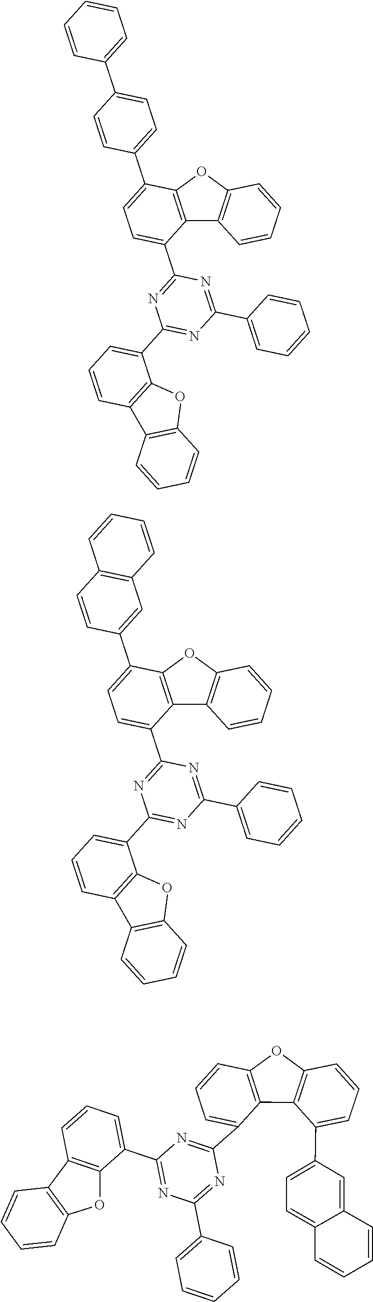

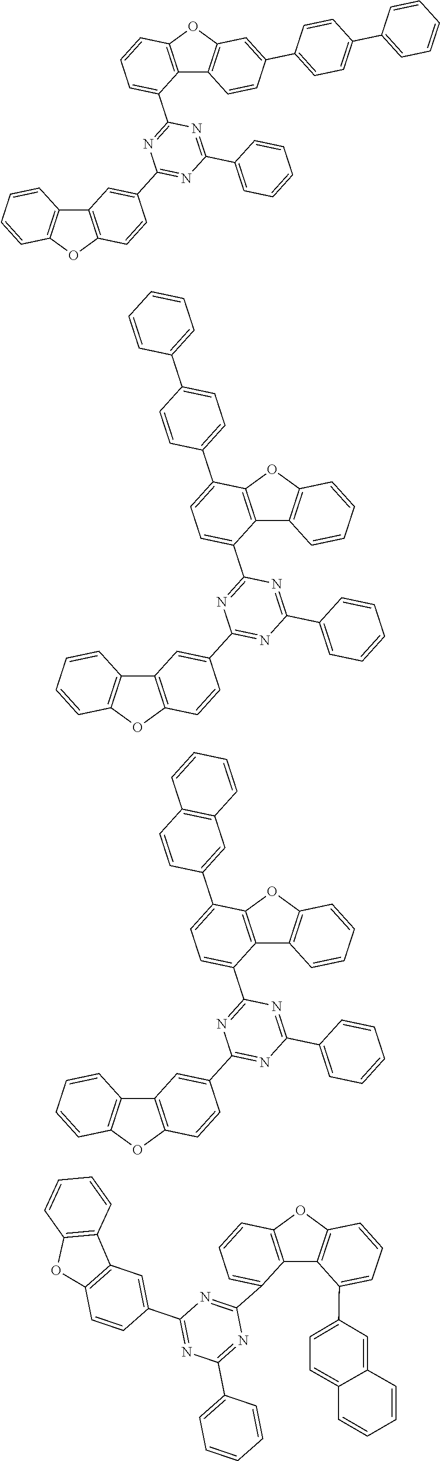

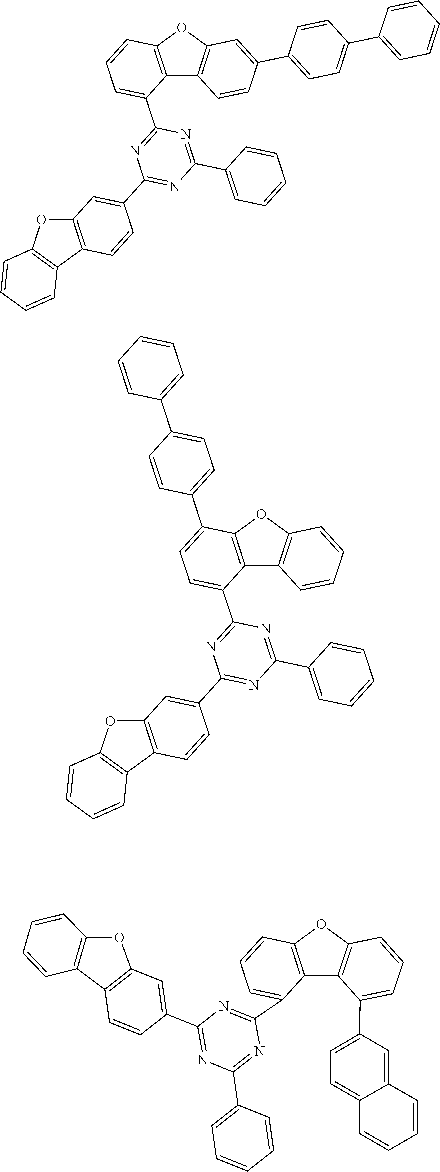 [SMILES: c1ccc(-c2ccc(-c3ccc4c(c3)oc3cccc(-c5nc(-c6ccccc6)nc(-c6ccc7c(c6)oc6ccccc67)n5)c34)cc2)cc1 ; c1ccc(-c2ccc(-c3ccc(-c4nc(-c5ccccc5)nc(-c5ccc6c(c5)oc5ccccc56)n4)c4c3oc3ccccc34)cc2)cc1 ; c1ccc(-c2nc(-c3ccc4c(c3)oc3ccccc34)nc(-c3cccc4oc5cccc(-c6ccc7ccccc7c6)c5c34)n2)cc1]

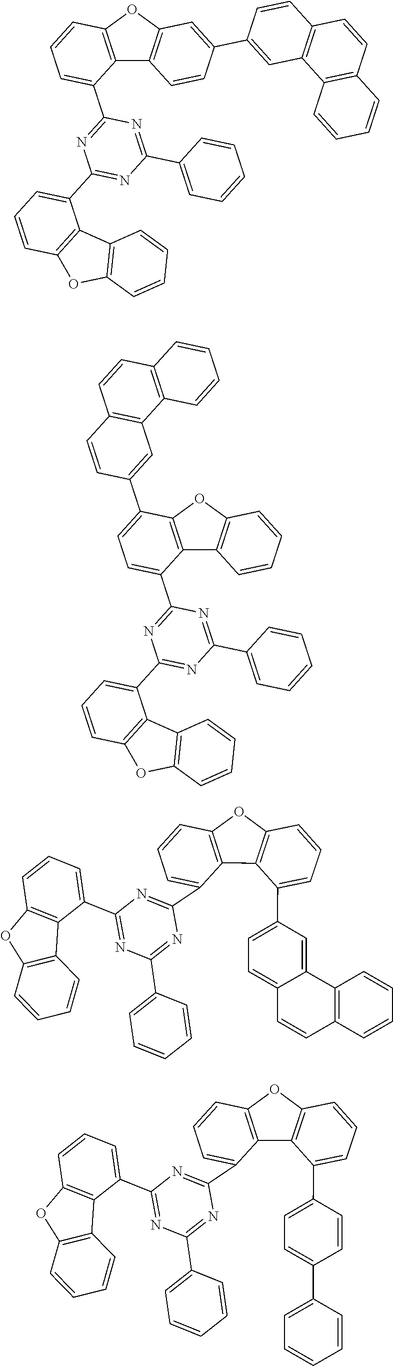

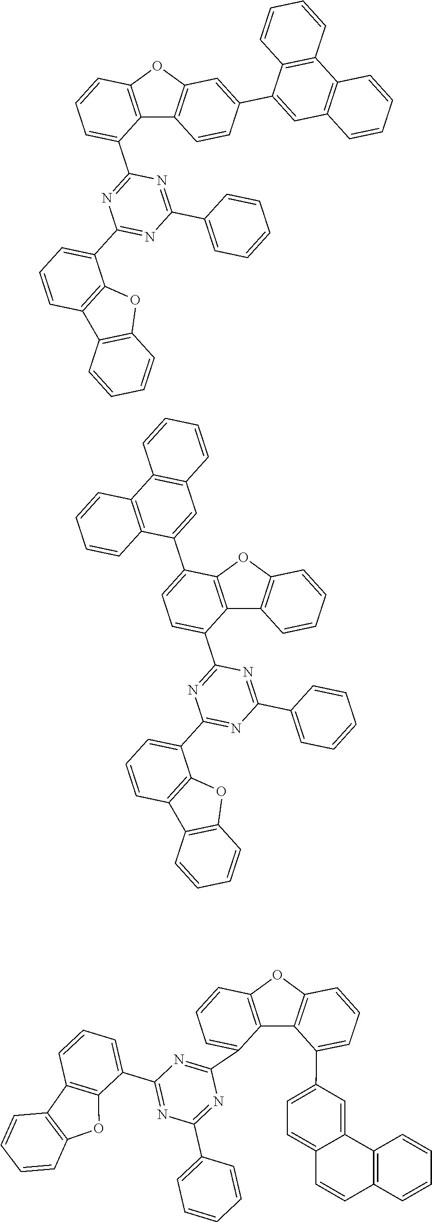

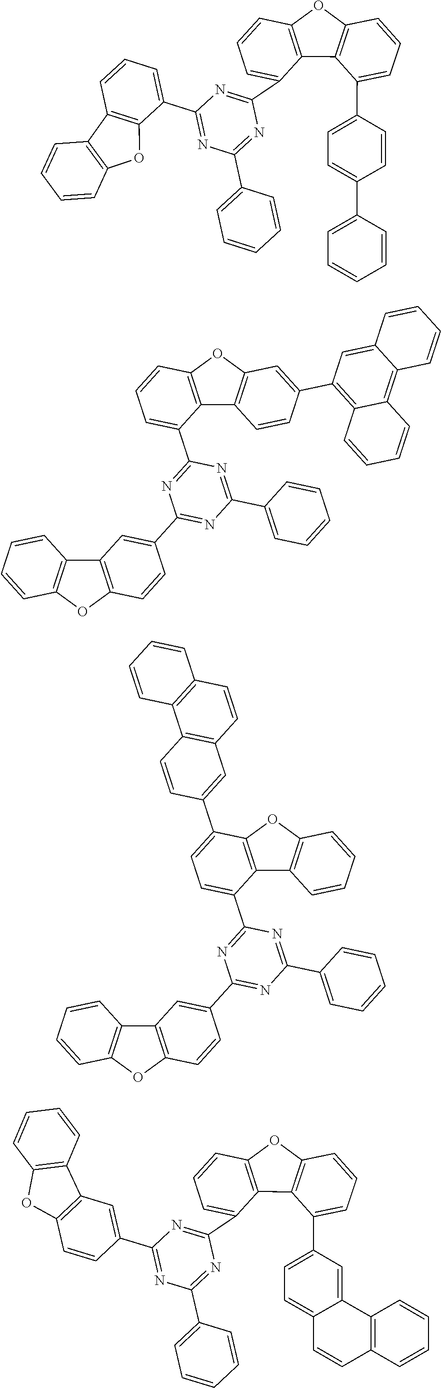

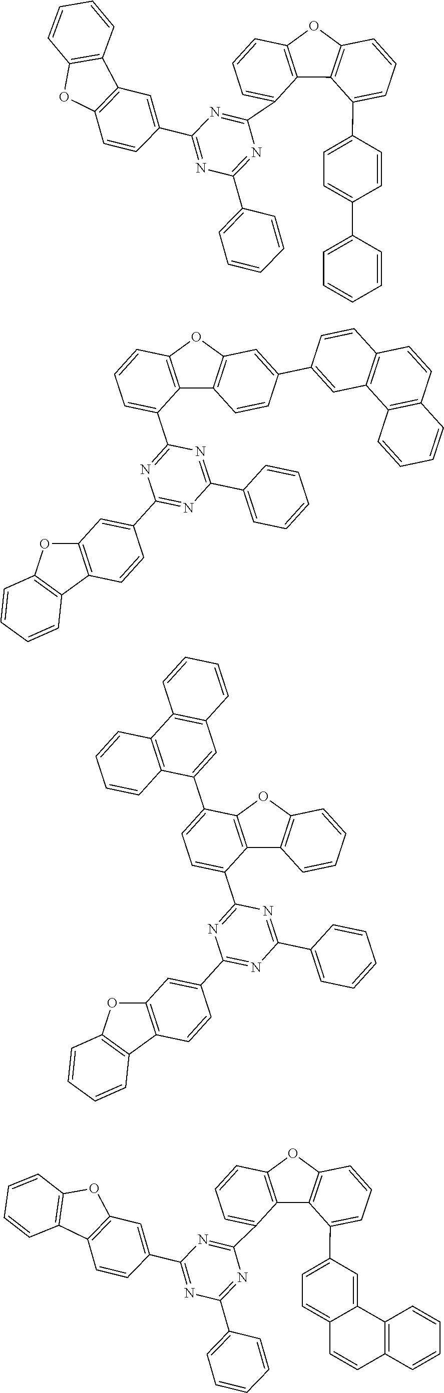

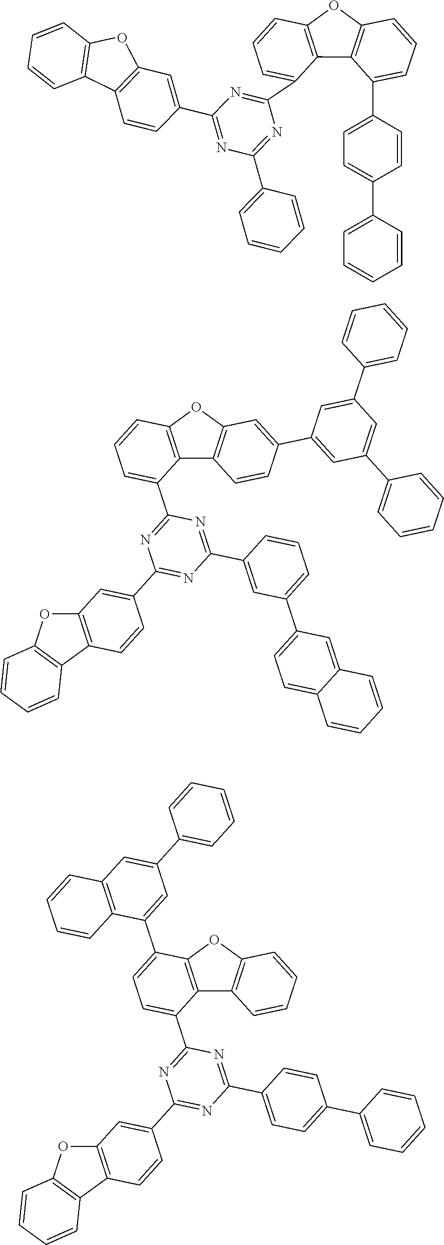

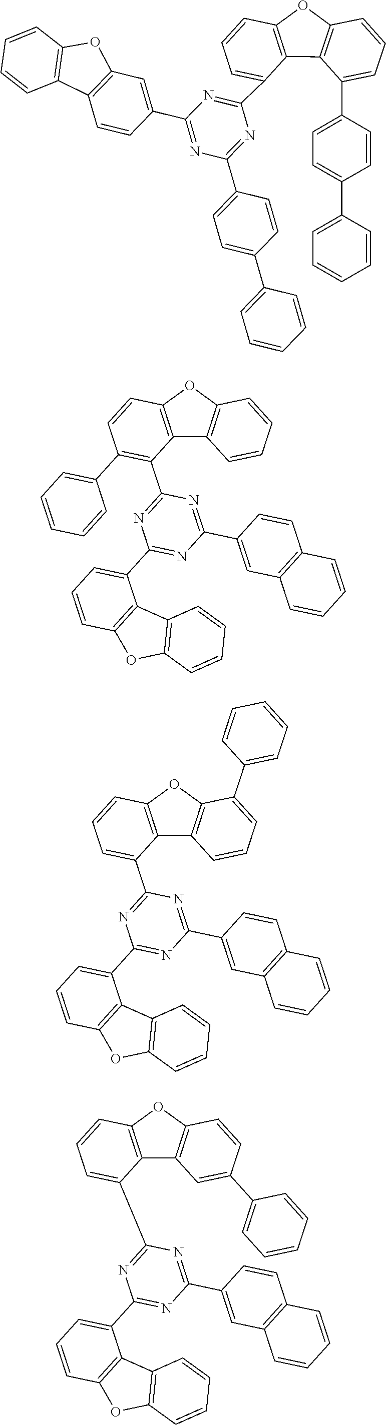

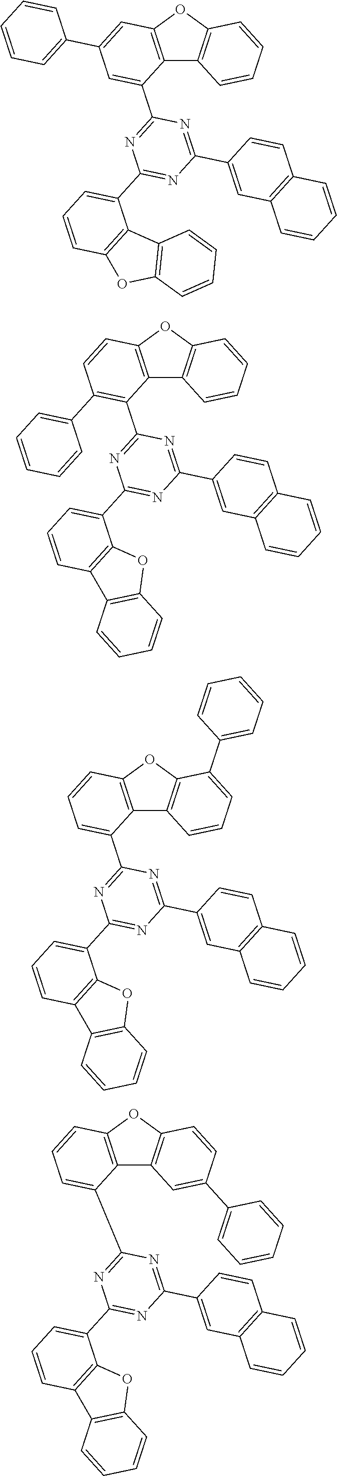

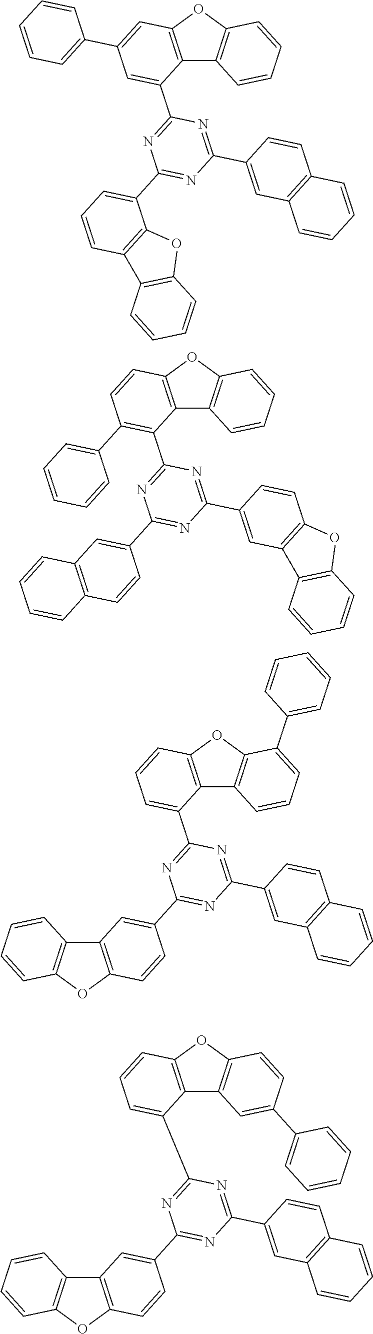

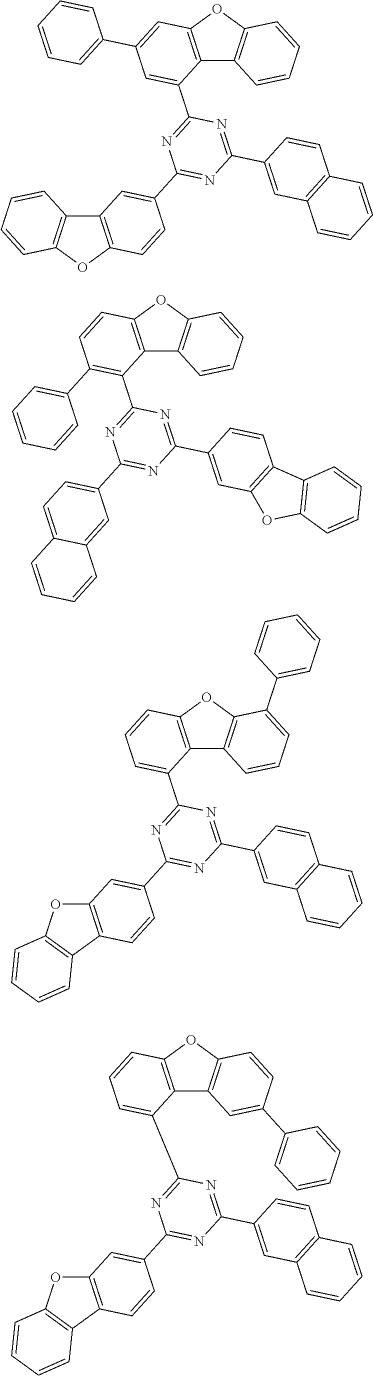 [SMILES: c1ccc(-c2cc(-c3nc(-c4ccc5ccccc5c4)nc(-c4ccc5oc6ccccc6c5c4)n3)c3c(c2)oc2ccccc23)cc1 ; c1ccc(-c2ccc3oc4ccccc4c3c2-c2nc(-c3ccc4ccccc4c3)nc(-c3ccc4c(c3)oc3ccccc34)n2)cc1 ; c1ccc(-c2cccc3c2oc2cccc(-c4nc(-c5ccc6ccccc6c5)nc(-c5ccc6c(c5)oc5ccccc56)n4)c23)cc1 ; c1ccc(-c2ccc3oc4cccc(-c5nc(-c6ccc7ccccc7c6)nc(-c6ccc7c(c6)oc6ccccc67)n5)c4c3c2)cc1]

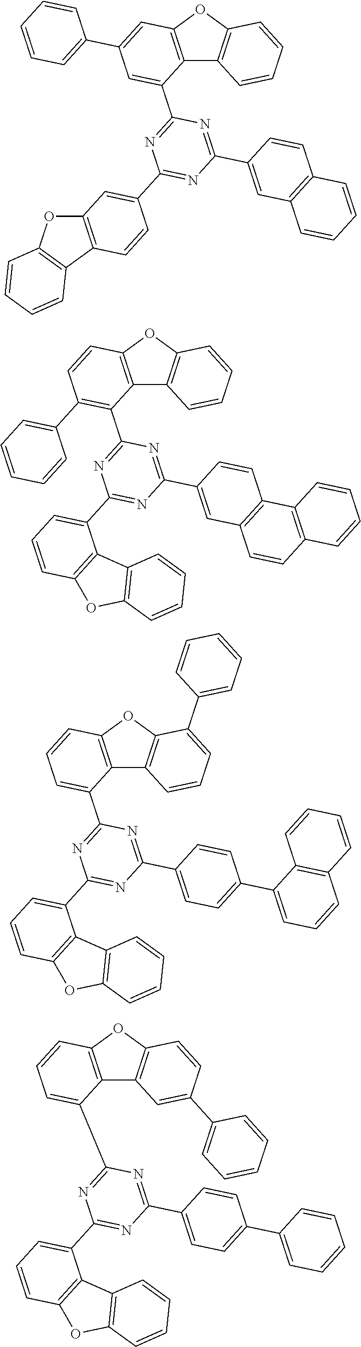

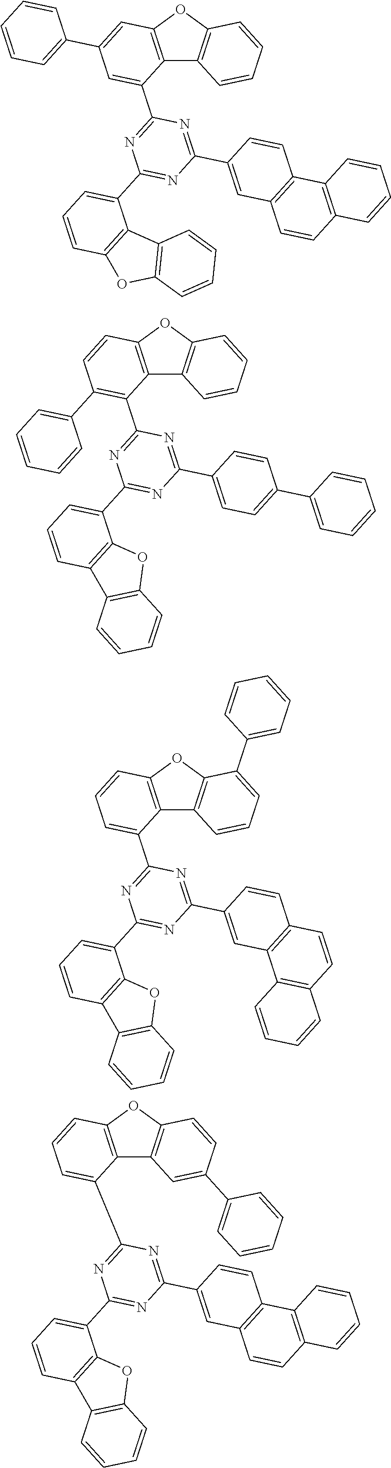

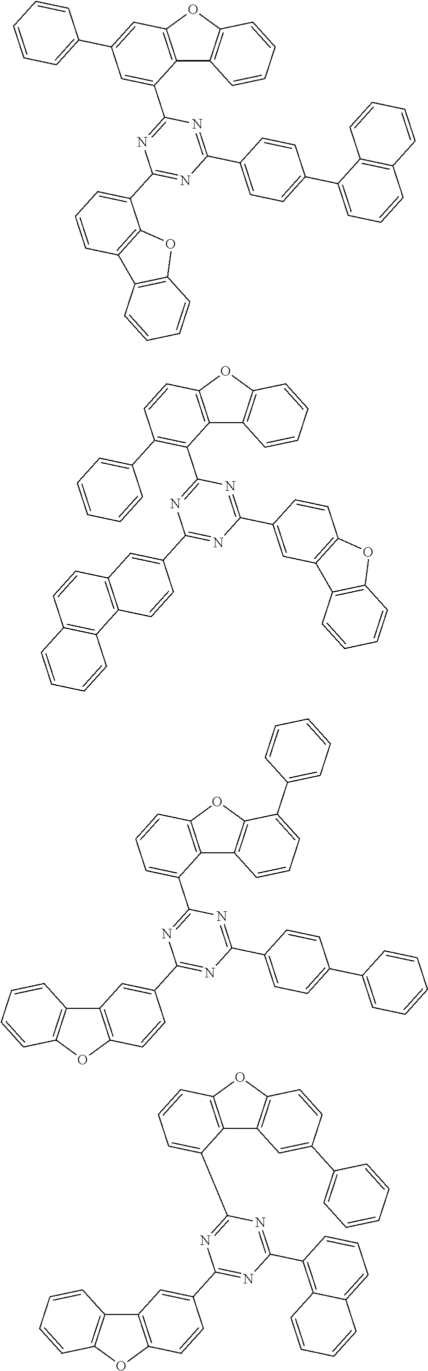 [SMILES: c1ccc(-c2cc(-c3nc(-c4ccc(-c5cccc6ccccc56)cc4)nc(-c4cccc5c4oc4ccccc45)n3)c3c(c2)oc2ccccc23)cc1 ; c1ccc(-c2ccc3oc4ccccc4c3c2-c2nc(-c3ccc4c(ccc5ccccc54)c3)nc(-c3ccc4oc5ccccc5c4c3)n2)cc1 ; c1ccc(-c2ccc(-c3nc(-c4ccc5oc6ccccc6c5c4)nc(-c4cccc5oc6c(-c7ccccc7)cccc6c45)n3)cc2)cc1 ; c1ccc(-c2ccc3oc4cccc(-c5nc(-c6ccc7oc8ccccc8c7c6)nc(-c6cccc7ccccc67)n5)c4c3c2)cc1]

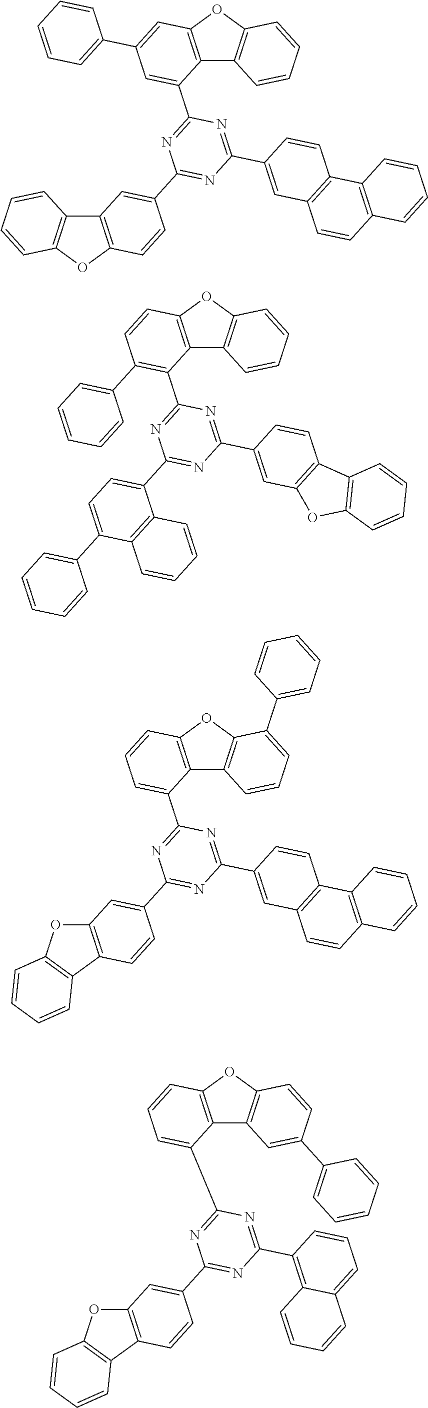 [SMILES: c1ccc(-c2cc(-c3nc(-c4ccc5c(ccc6ccccc65)c4)nc(-c4ccc5oc6ccccc6c5c4)n3)c3c(c2)oc2ccccc23)cc1 ; c1ccc(-c2ccc3oc4ccccc4c3c2-c2nc(-c3ccc4c(c3)oc3ccccc34)nc(-c3ccc(-c4ccccc4)c4ccccc34)n2)cc1 ; c1ccc(-c2cccc3c2oc2cccc(-c4nc(-c5ccc6c(ccc7ccccc76)c5)nc(-c5ccc6c(c5)oc5ccccc56)n4)c23)cc1 ; c1ccc(-c2ccc3oc4cccc(-c5nc(-c6ccc7c(c6)oc6ccccc67)nc(-c6cccc7ccccc67)n5)c4c3c2)cc1]

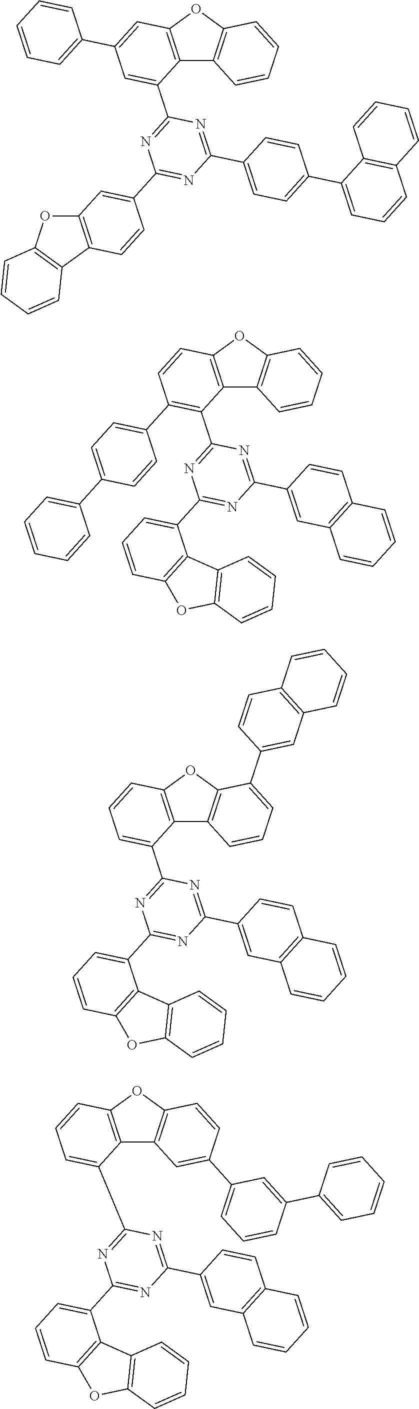

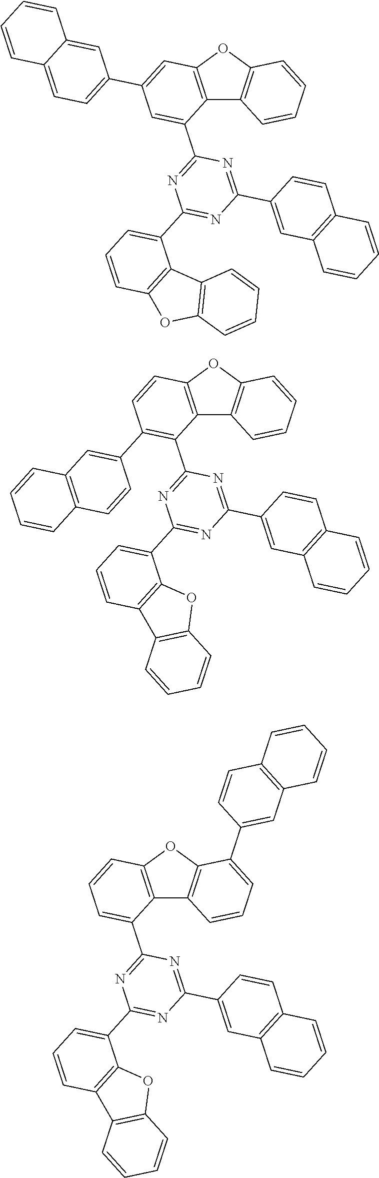

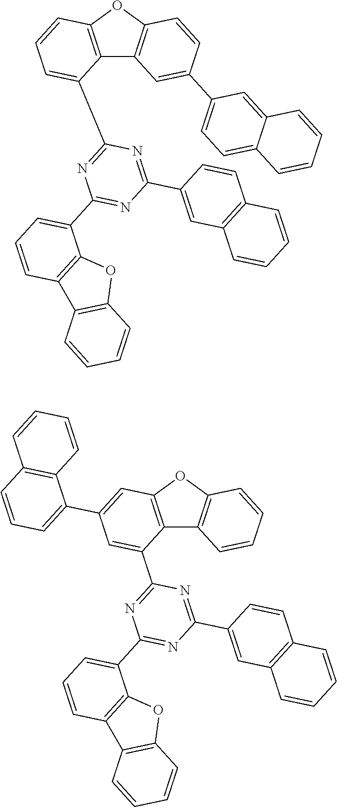 [SMILES: c1ccc2cc(-c3ccc4oc5cccc(-c6nc(-c7ccc8ccccc8c7)nc(-c7cccc8c7oc7ccccc78)n6)c5c4c3)ccc2c1 ; c1ccc2cc(-c3nc(-c4cccc5c4oc4ccccc45)nc(-c4cc(-c5cccc6ccccc56)cc5oc6ccccc6c45)n3)ccc2c1]

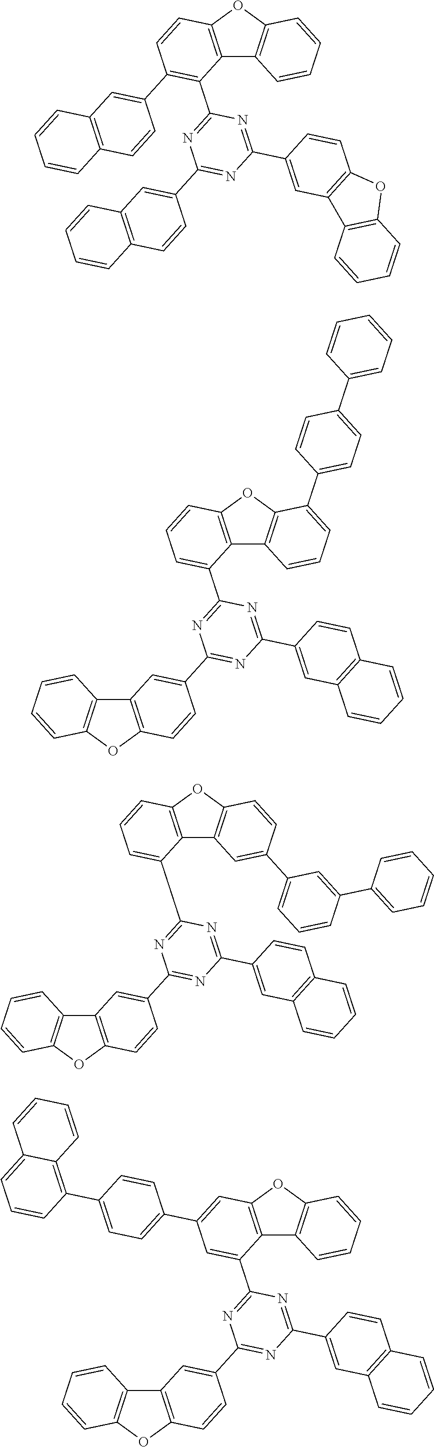

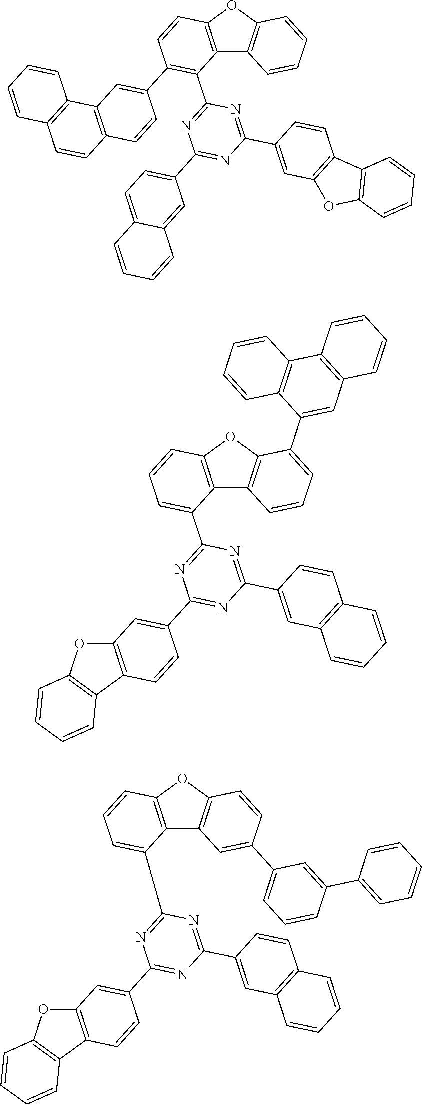 [SMILES: c1ccc2cc(-c3nc(-c4ccc5c(c4)oc4ccccc45)nc(-c4c(-c5ccc6ccc7ccccc7c6c5)ccc5oc6ccccc6c45)n3)ccc2c1 ; c1ccc2cc(-c3nc(-c4ccc5c(c4)oc4ccccc45)nc(-c4cccc5oc6c(-c7cc8ccccc8c8ccccc78)cccc6c45)n3)ccc2c1 ; c1ccc(-c2cccc(-c3ccc4oc5cccc(-c6nc(-c7ccc8ccccc8c7)nc(-c7ccc8c(c7)oc7ccccc78)n6)c5c4c3)c2)cc1]

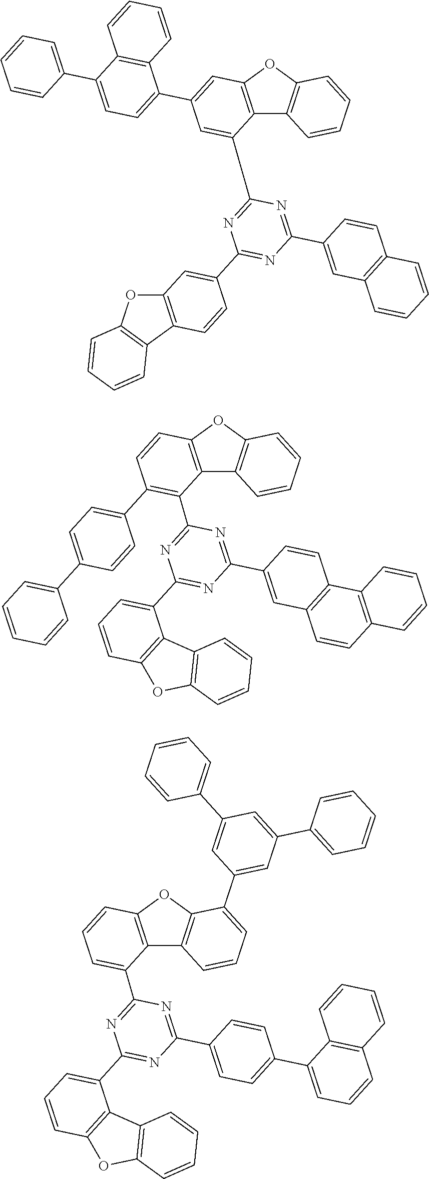

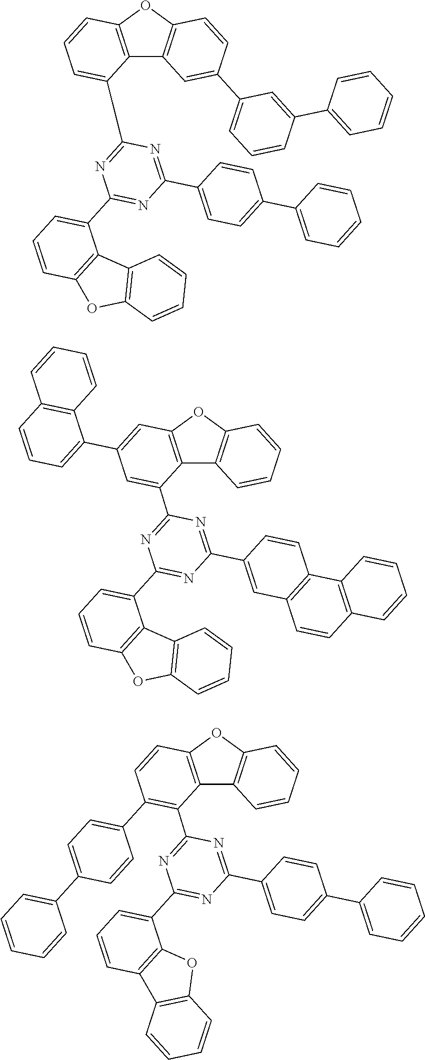

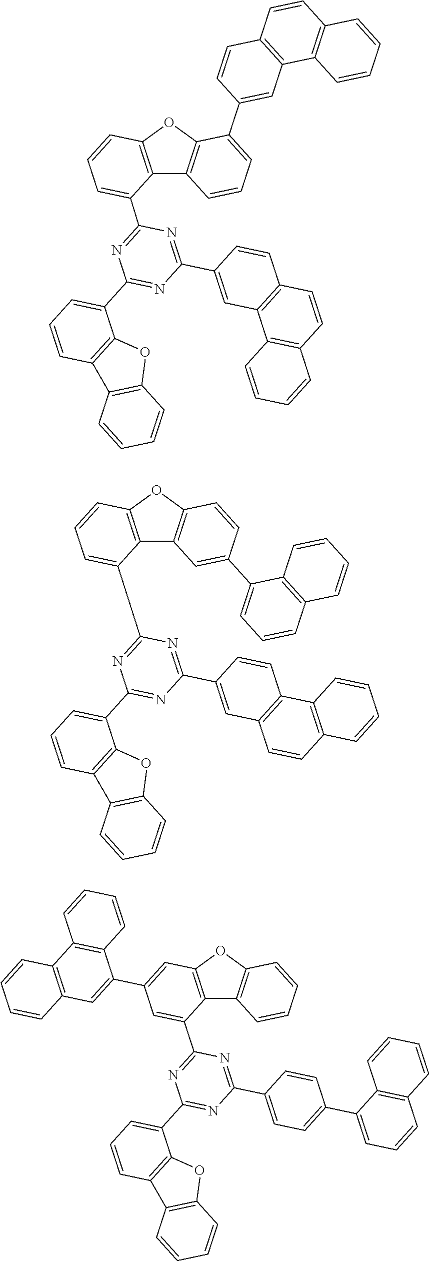

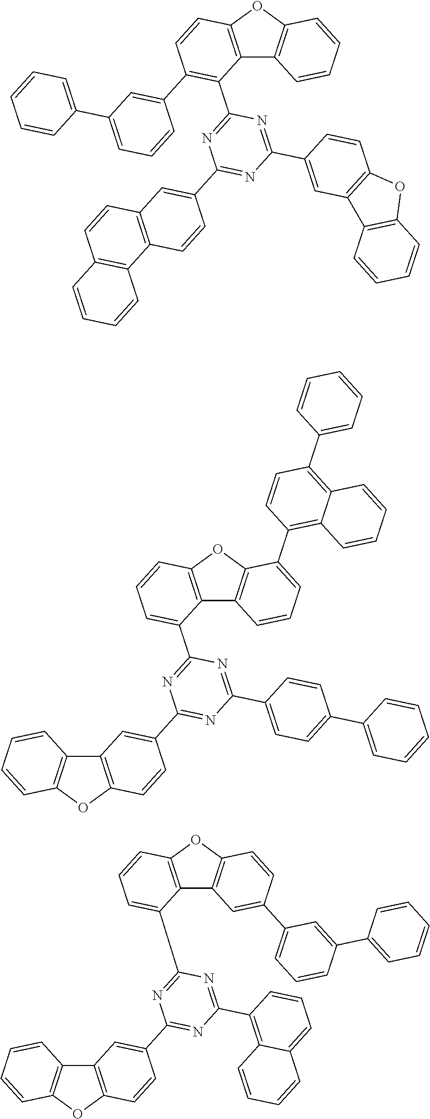 [SMILES: c1ccc(-c2cccc(-c3ccc4oc5ccccc5c4c3-c3nc(-c4ccc5c(ccc6ccccc65)c4)nc(-c4ccc5oc6ccccc6c5c4)n3)c2)cc1 ; c1ccc(-c2ccc(-c3nc(-c4ccc5oc6ccccc6c5c4)nc(-c4cccc5oc6c(-c7ccc(-c8ccccc8)c8ccccc78)cccc6c45)n3)cc2)cc1 ; c1ccc(-c2cccc(-c3ccc4oc5cccc(-c6nc(-c7ccc8oc9ccccc9c8c7)nc(-c7cccc8ccccc78)n6)c5c4c3)c2)cc1]

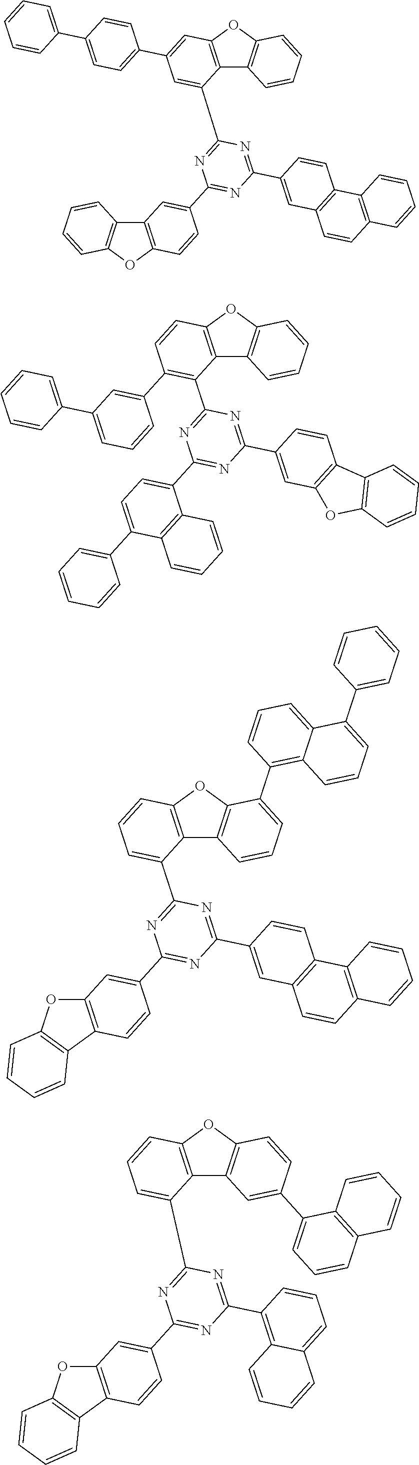 [SMILES: c1ccc(-c2ccc(-c3cc(-c4nc(-c5ccc6c(ccc7ccccc76)c5)nc(-c5ccc6oc7ccccc7c6c5)n4)c4c(c3)oc3ccccc34)cc2)cc1 ; c1ccc(-c2cccc(-c3ccc4oc5ccccc5c4c3-c3nc(-c4ccc5c(c4)oc4ccccc45)nc(-c4ccc(-c5ccccc5)c5ccccc45)n3)c2)cc1 ; c1ccc(-c2cccc3c(-c4cccc5c4oc4cccc(-c6nc(-c7ccc8c(ccc9ccccc98)c7)nc(-c7ccc8c(c7)oc7ccccc78)n6)c45)cccc23)cc1 ; c1ccc2c(-c3ccc4oc5cccc(-c6nc(-c7ccc8c(c7)oc7ccccc78)nc(-c7cccc8ccccc78)n6)c5c4c3)cccc2c1]

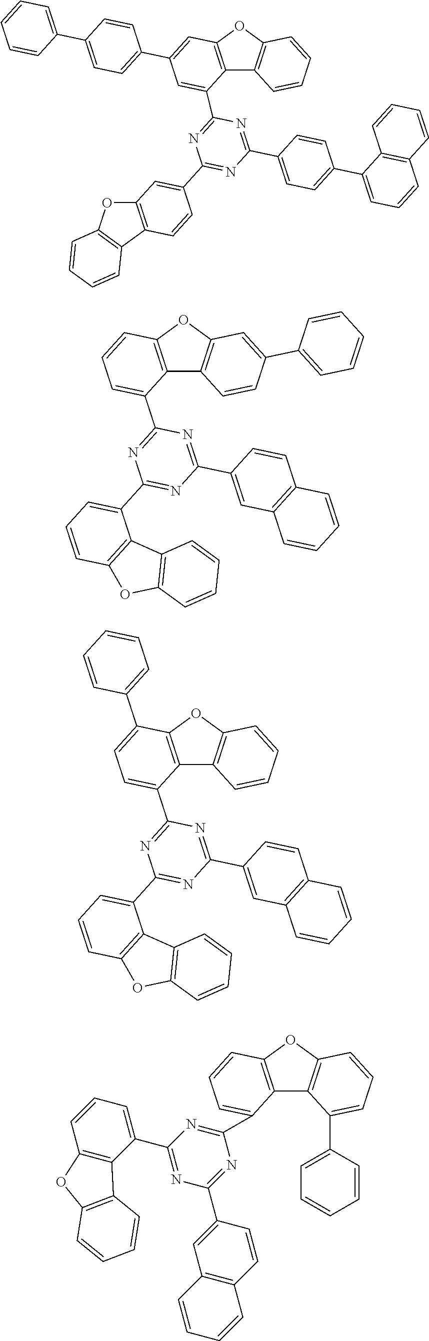

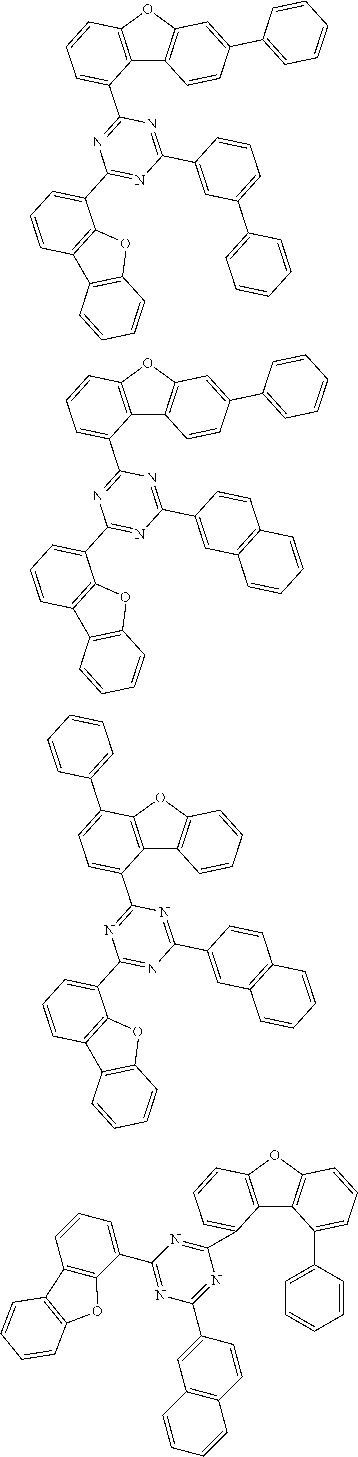

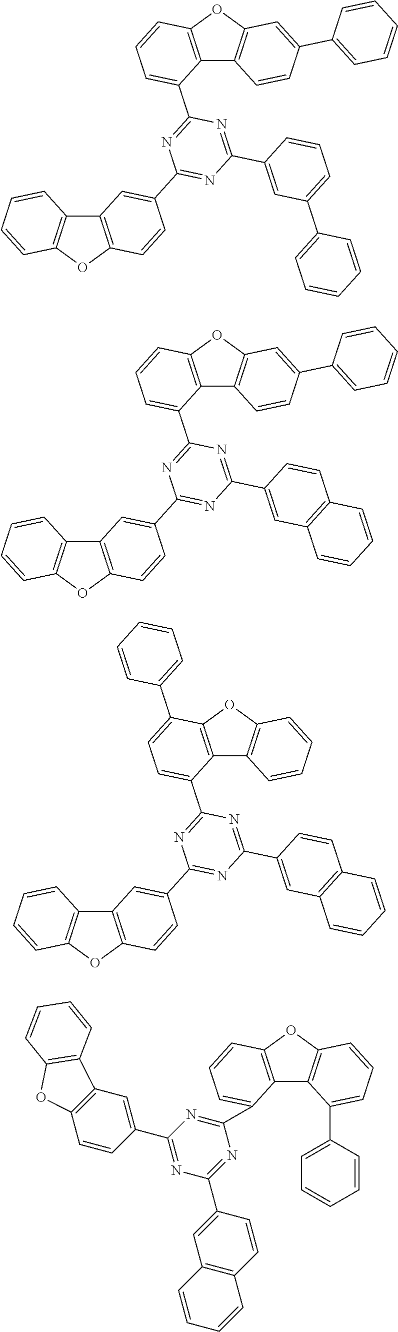

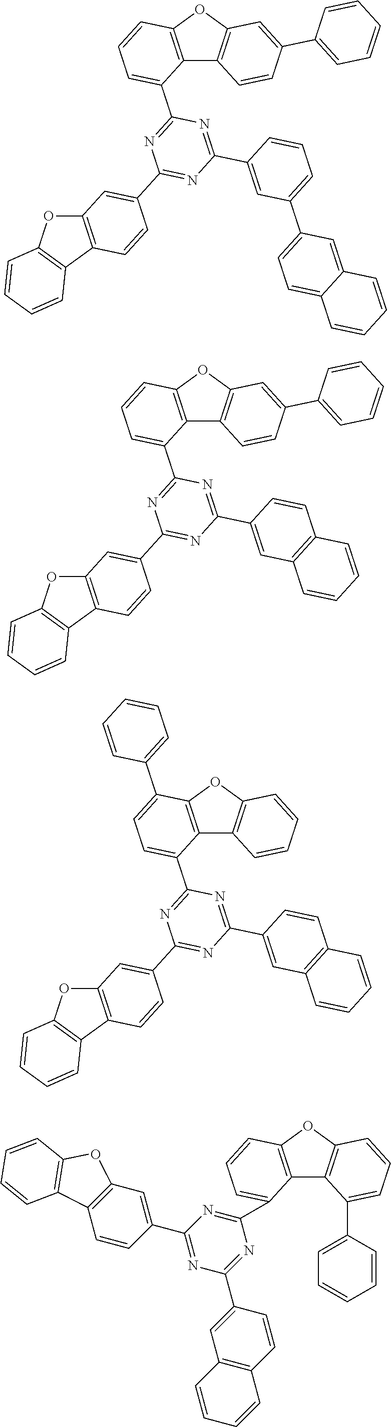

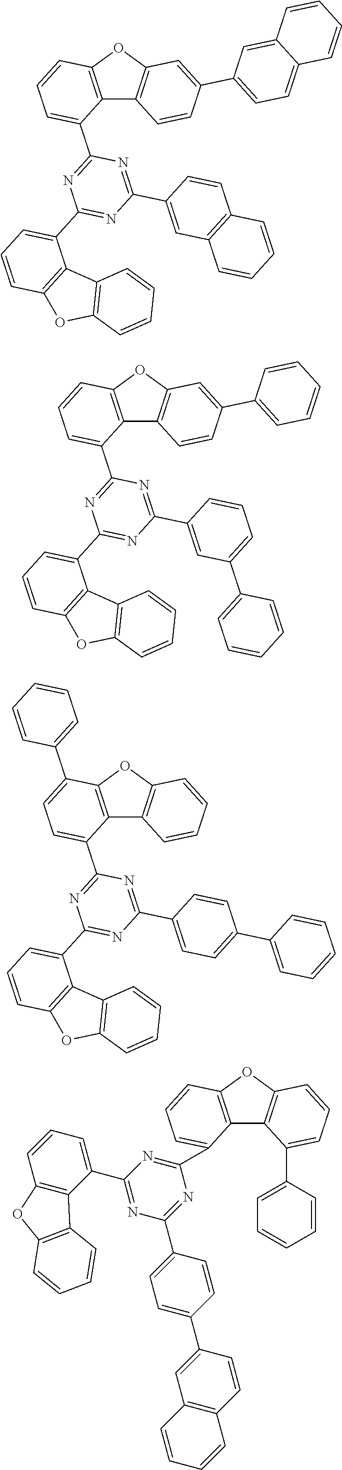

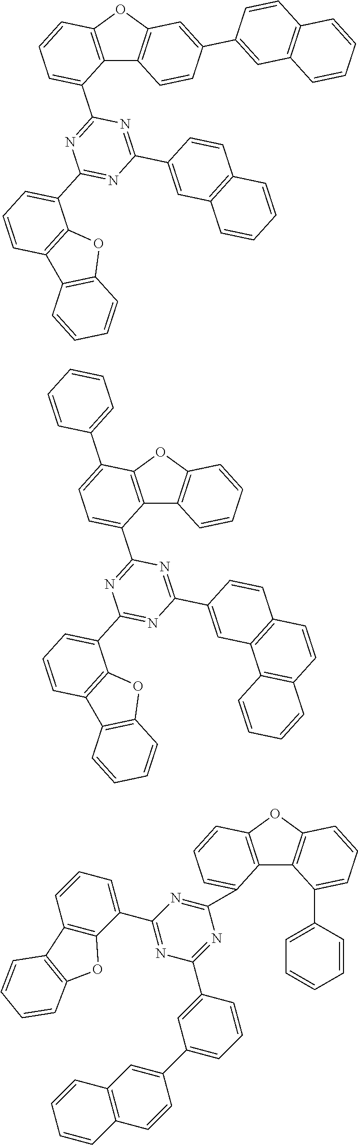

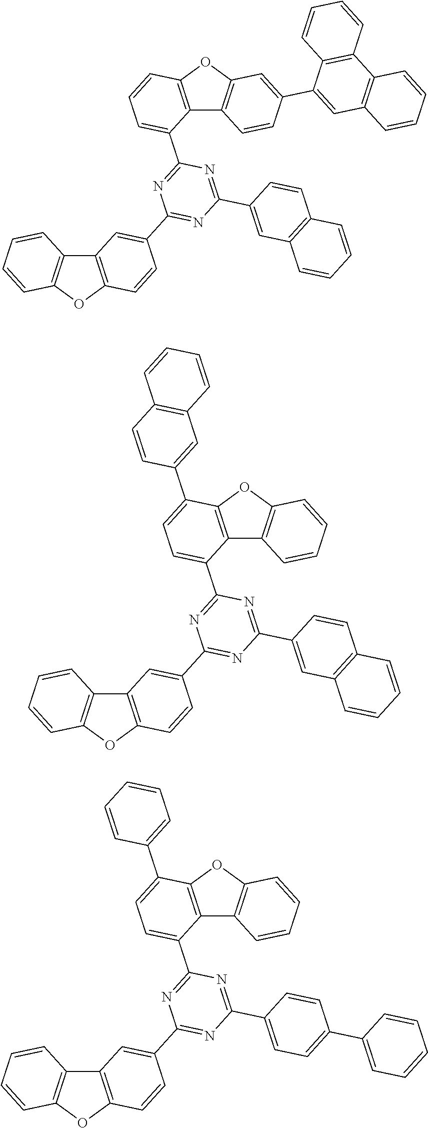

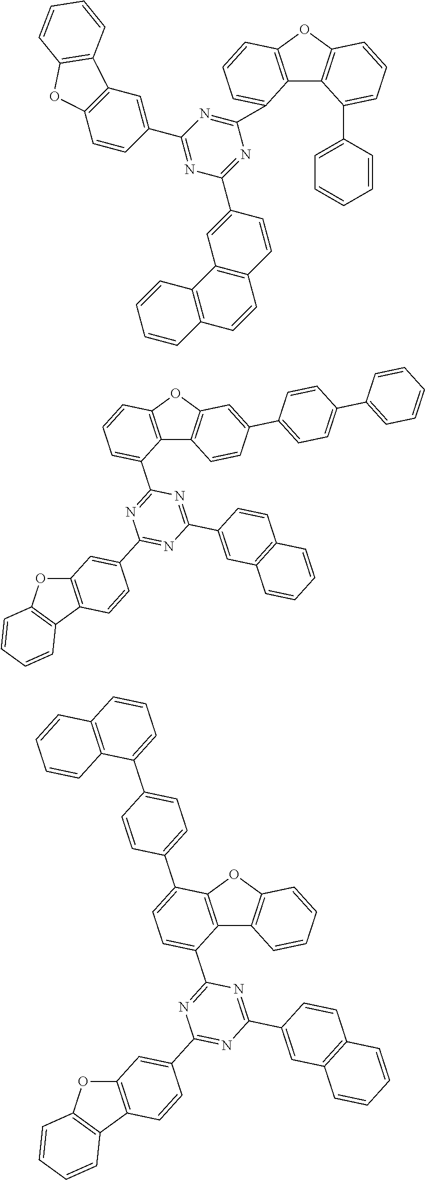 [SMILES: c1ccc(-c2cccc3oc4cccc(-c5nc(-c6ccc7ccc8ccccc8c7c6)nc(-c6ccc7oc8ccccc8c7c6)n5)c4c23)cc1 ; c1ccc(-c2ccc(-c3ccc4c(c3)oc3cccc(-c5nc(-c6ccc7ccccc7c6)nc(-c6ccc7c(c6)oc6ccccc67)n5)c34)cc2)cc1 ; c1ccc2cc(-c3nc(-c4ccc5c(c4)oc4ccccc45)nc(-c4ccc(-c5ccc(-c6cccc7ccccc67)cc5)c5oc6ccccc6c45)n3)ccc2c1]

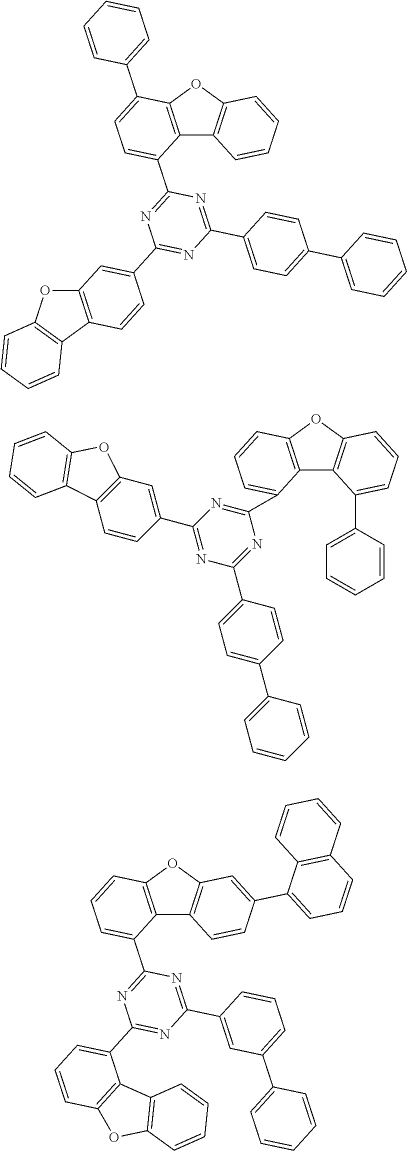

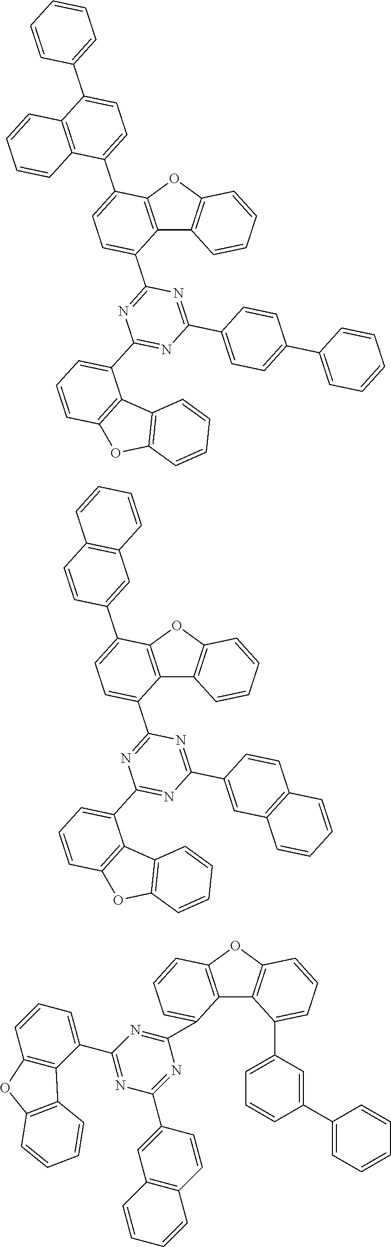

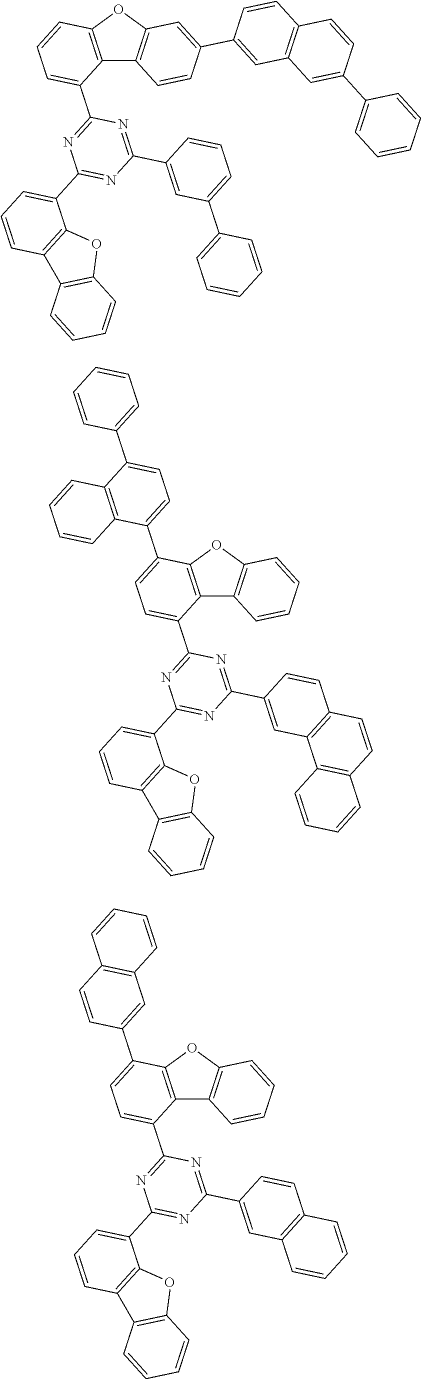

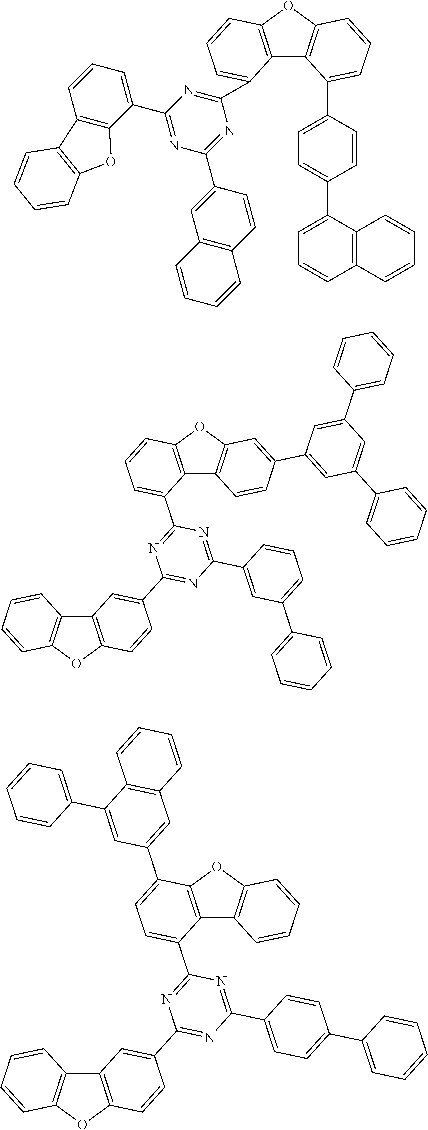 [SMILES: c1ccc2cc(-c3nc(-c4cccc5c4oc4ccccc45)nc(-c4cccc5oc6cccc(-c7ccc(-c8cccc9ccccc89)cc7)c6c45)n3)ccc2c1 ; c1ccc(-c2cc(-c3ccccc3)cc(-c3ccc4c(c3)oc3cccc(-c5nc(-c6cccc(-c7ccccc7)c6)nc(-c6ccc7oc8ccccc8c7c6)n5)c34)c2)cc1 ; c1ccc(-c2ccc(-c3nc(-c4ccc5oc6ccccc6c5c4)nc(-c4ccc(-c5cc(-c6ccccc6)c6ccccc6c5)c5oc6ccccc6c45)n3)cc2)cc1]

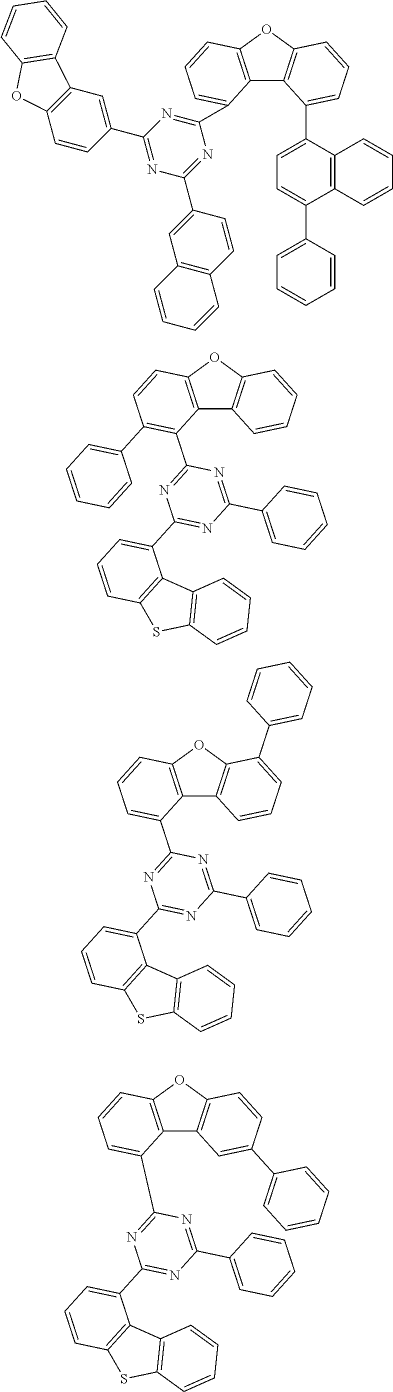

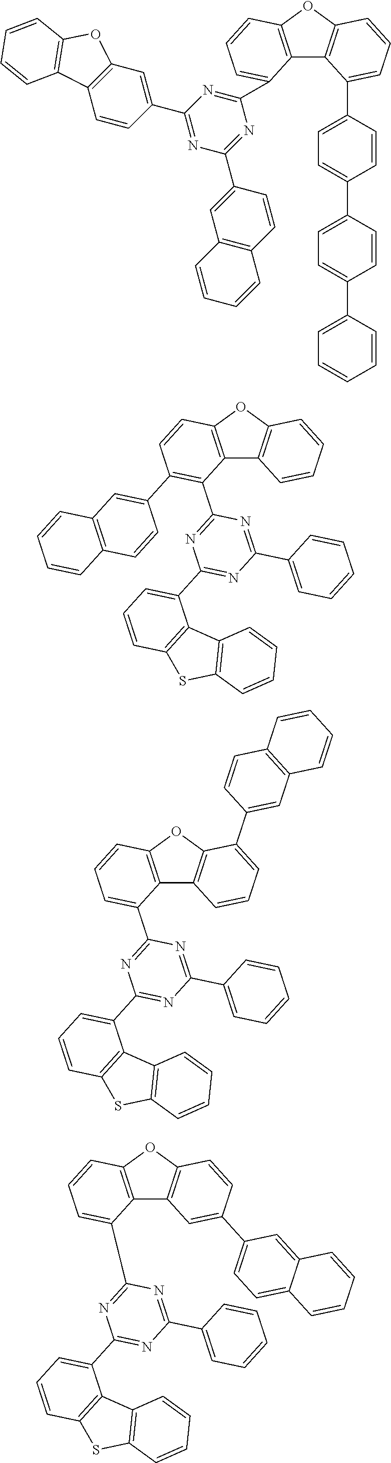

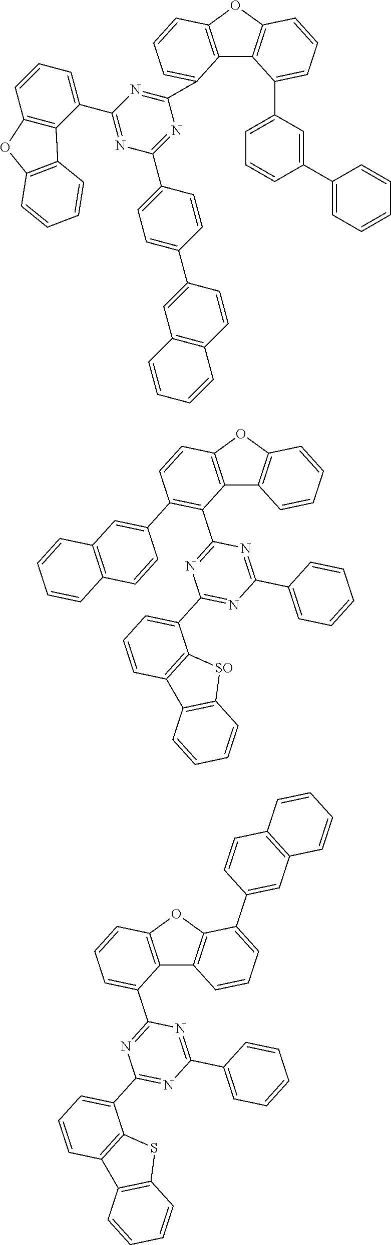

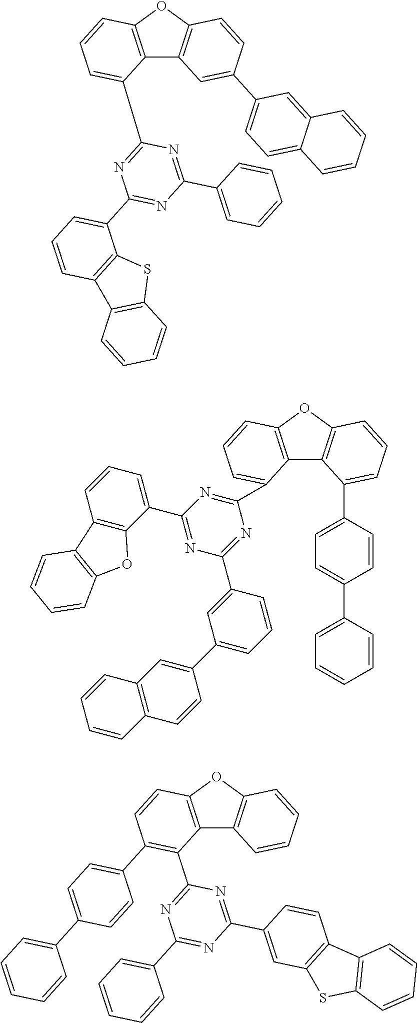 [SMILES: c1ccc(-c2nc(-c3cccc4c3sc3ccccc34)nc(-c3cccc4oc5ccc(-c6ccc7ccccc7c6)cc5c34)n2)cc1 ; c1ccc(-c2ccc(-c3cccc4oc5cccc(-c6nc(-c7cccc(-c8ccc9ccccc9c8)c7)nc(-c7cccc8c7oc7ccccc78)n6)c5c34)cc2)cc1 ; c1ccc(-c2ccc(-c3ccc4oc5ccccc5c4c3-c3nc(-c4ccccc4)nc(-c4ccc5c(c4)sc4ccccc45)n3)cc2)cc1]

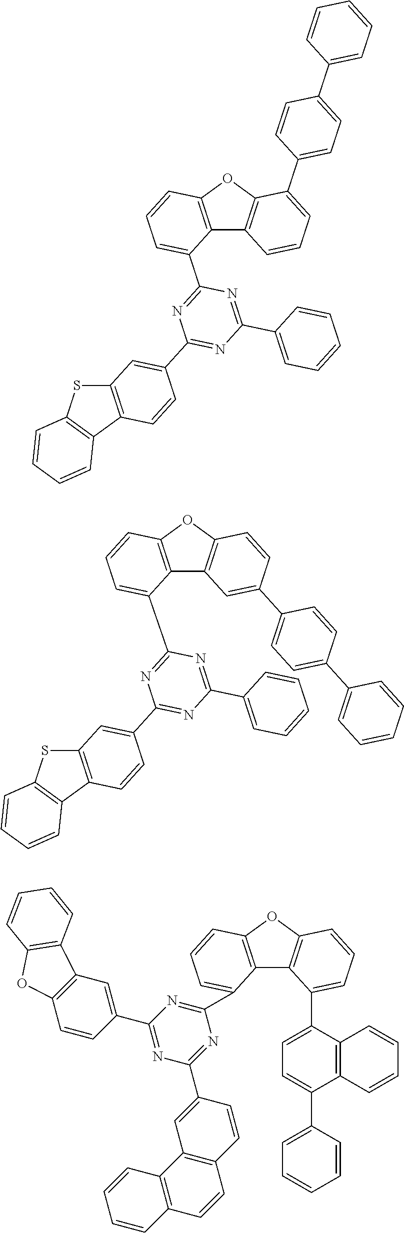

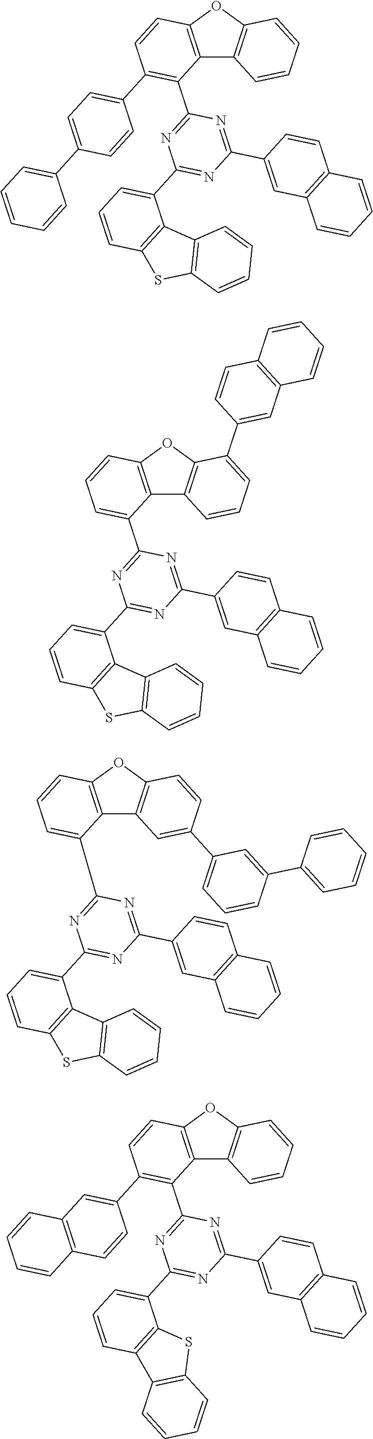

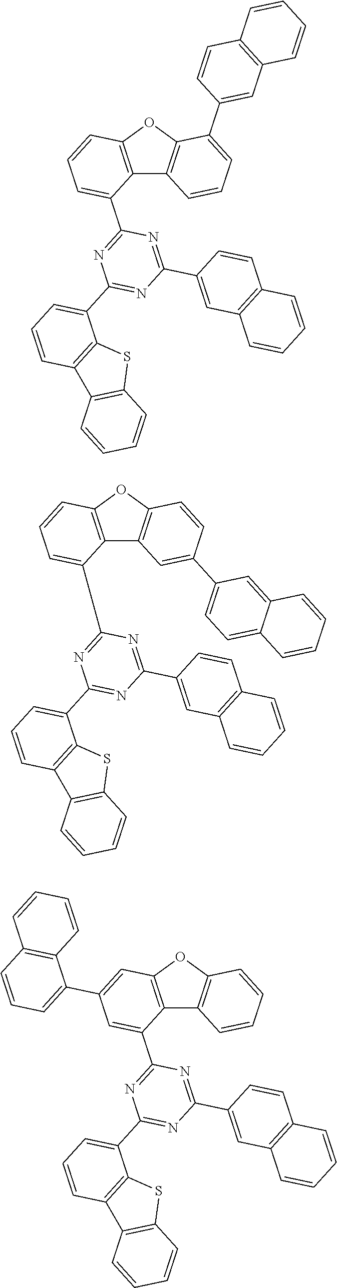

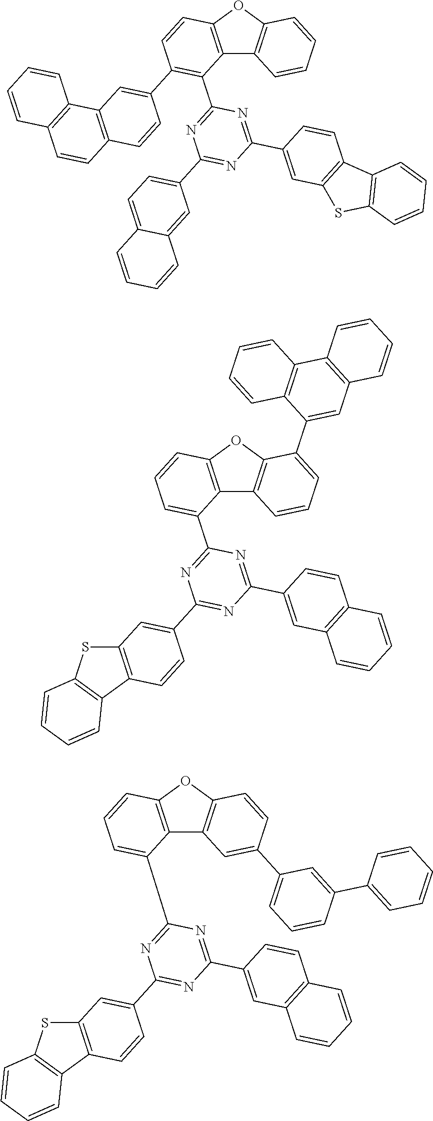 [SMILES: c1ccc2cc(-c3nc(-c4ccc5c(c4)sc4ccccc45)nc(-c4c(-c5ccc6ccc7ccccc7c6c5)ccc5oc6ccccc6c45)n3)ccc2c1 ; c1ccc2cc(-c3nc(-c4ccc5c(c4)sc4ccccc45)nc(-c4cccc5oc6c(-c7cc8ccccc8c8ccccc78)cccc6c45)n3)ccc2c1 ; c1ccc(-c2cccc(-c3ccc4oc5cccc(-c6nc(-c7ccc8ccccc8c7)nc(-c7ccc8c(c7)sc7ccccc78)n6)c5c4c3)c2)cc1]

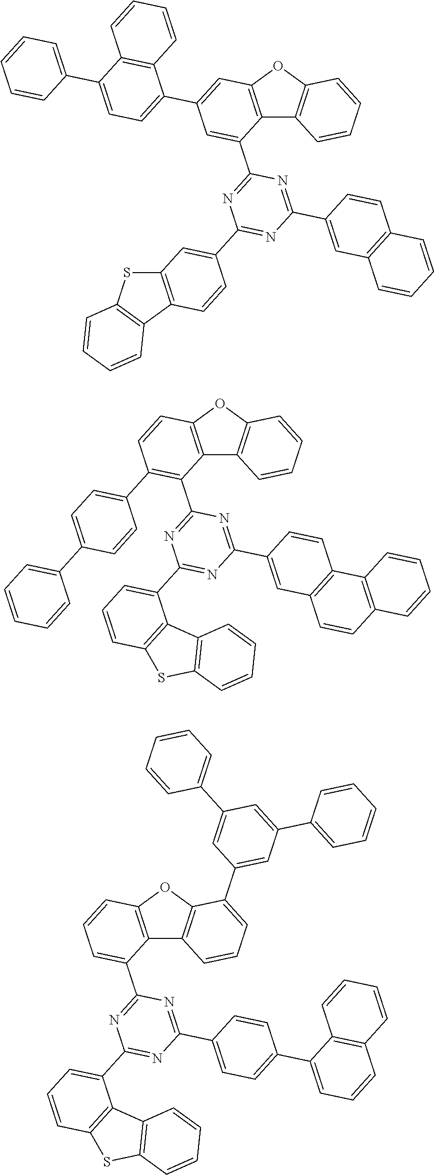 [SMILES: c1ccc(-c2ccc(-c3cc(-c4nc(-c5ccc6ccccc6c5)nc(-c5ccc6c(c5)sc5ccccc56)n4)c4c(c3)oc3ccccc34)c3ccccc23)cc1 ; c1ccc(-c2ccc(-c3ccc4oc5ccccc5c4c3-c3nc(-c4ccc5c(ccc6ccccc65)c4)nc(-c4cccc5sc6ccccc6c45)n3)cc2)cc1 ; c1ccc(-c2cc(-c3ccccc3)cc(-c3cccc4c3oc3cccc(-c5nc(-c6ccc(-c7cccc8ccccc78)cc6)nc(-c6cccc7sc8ccccc8c67)n5)c34)c2)cc1]

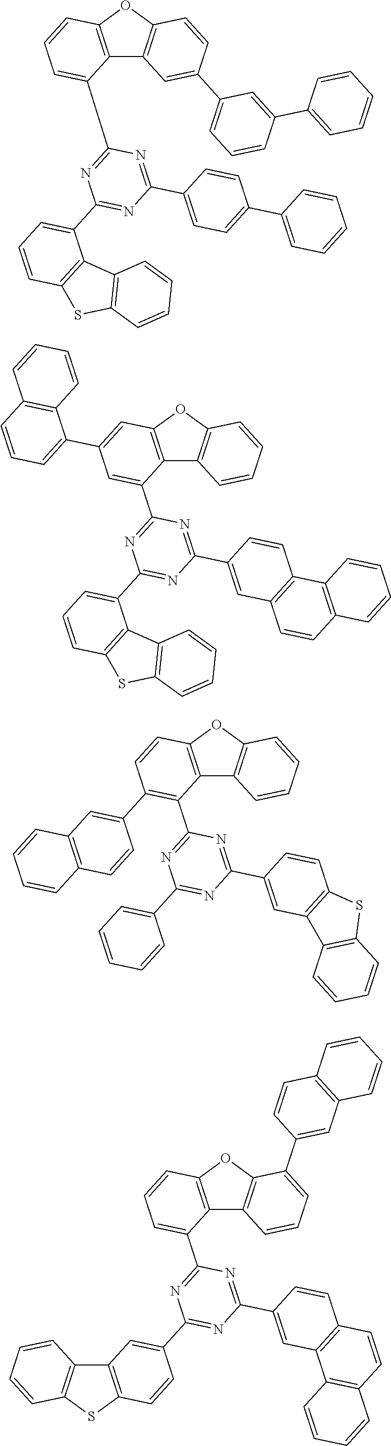

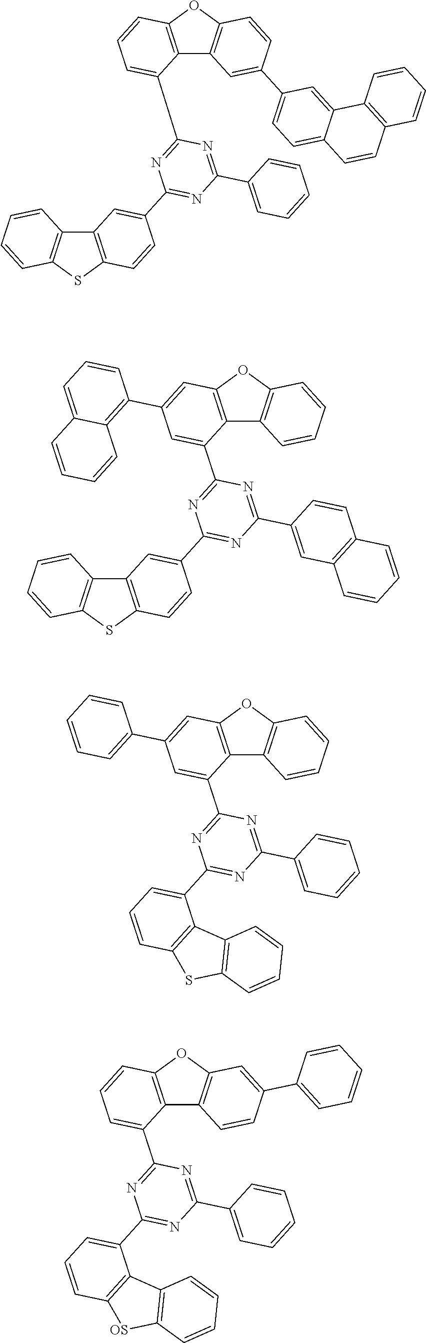

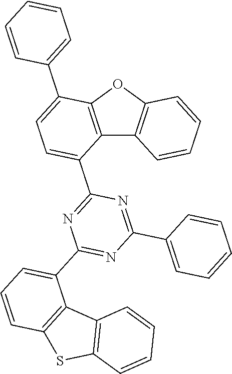 [SMILES: c1ccc(-c2nc(-c3cccc4sc5ccccc5c34)nc(-c3ccc(-c4ccccc4)c4oc5ccccc5c34)n2)cc1]

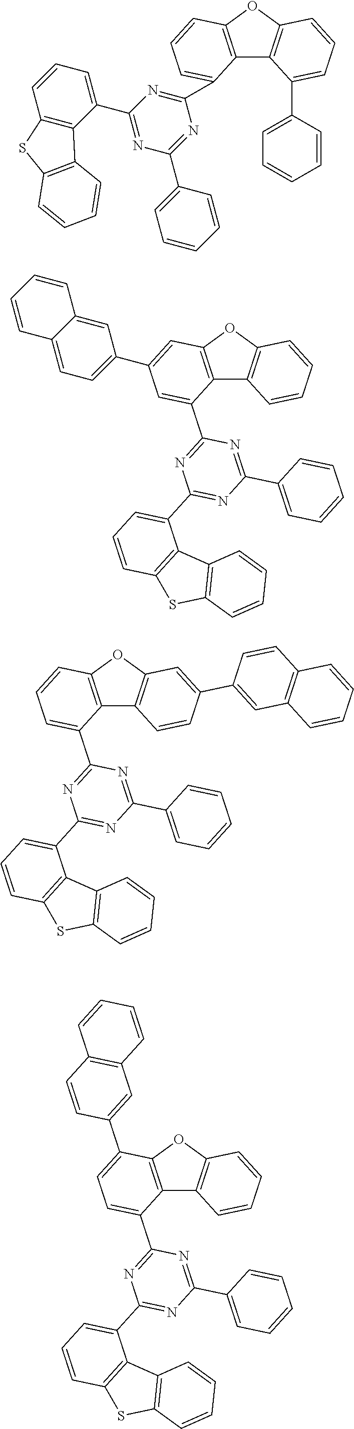

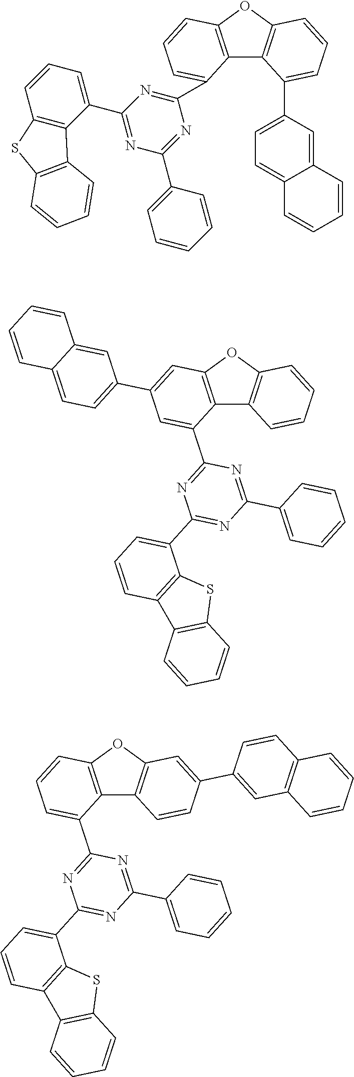 [SMILES: c1ccc(-c2nc(-c3cccc4sc5ccccc5c34)nc(-c3cccc4oc5cccc(-c6ccc7ccccc7c6)c5c34)n2)cc1 ; c1ccc(-c2nc(-c3cccc4c3sc3ccccc34)nc(-c3cc(-c4ccc5ccccc5c4)cc4oc5ccccc5c34)n2)cc1 ; c1ccc(-c2nc(-c3cccc4c3sc3ccccc34)nc(-c3cccc4oc5cc(-c6ccc7ccccc7c6)ccc5c34)n2)cc1]

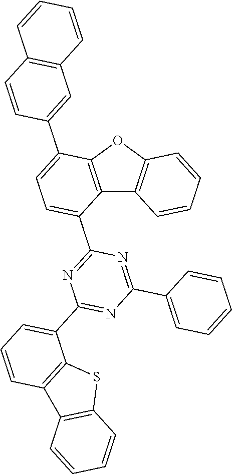 [SMILES: c1ccc(-c2nc(-c3cccc4c3sc3ccccc34)nc(-c3ccc(-c4ccc5ccccc5c4)c4oc5ccccc5c34)n2)cc1]

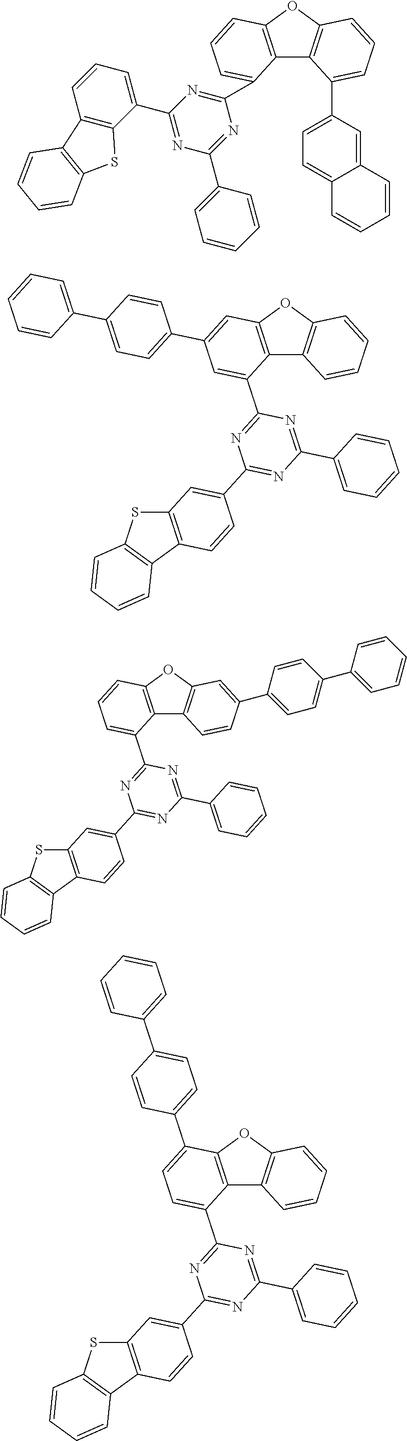 [SMILES: c1ccc(-c2nc(-c3cccc4c3sc3ccccc34)nc(-c3cccc4oc5cccc(-c6ccc7ccccc7c6)c5c34)n2)cc1 ; c1ccc(-c2ccc(-c3cc(-c4nc(-c5ccccc5)nc(-c5ccc6c(c5)sc5ccccc56)n4)c4c(c3)oc3ccccc34)cc2)cc1 ; c1ccc(-c2ccc(-c3ccc4c(c3)oc3cccc(-c5nc(-c6ccccc6)nc(-c6ccc7c(c6)sc6ccccc67)n5)c34)cc2)cc1 ; c1ccc(-c2ccc(-c3ccc(-c4nc(-c5ccccc5)nc(-c5ccc6c(c5)sc5ccccc56)n4)c4c3oc3ccccc34)cc2)cc1]

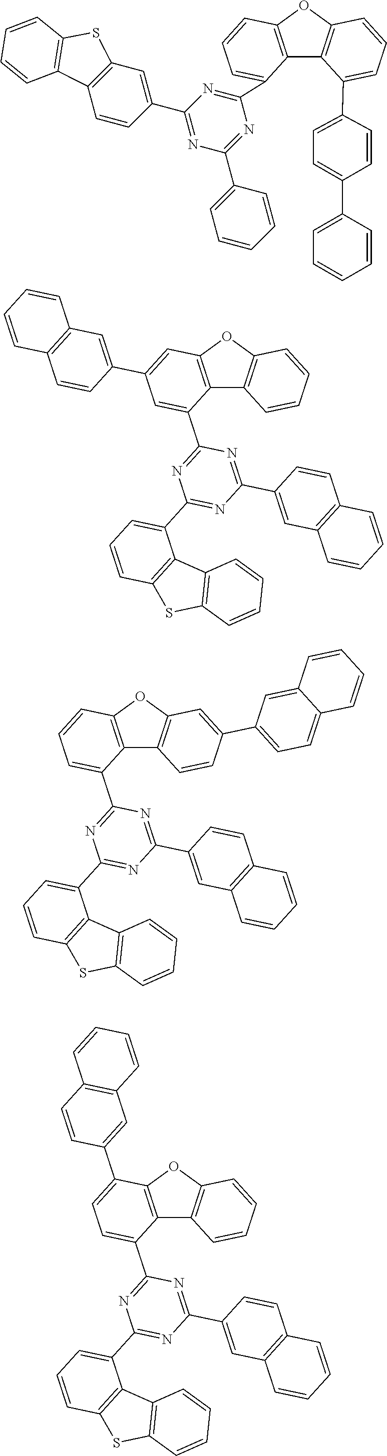 [SMILES: c1ccc(-c2ccc(-c3cccc4oc5cccc(-c6nc(-c7ccccc7)nc(-c7ccc8c(c7)sc7ccccc78)n6)c5c34)cc2)cc1 ; c1ccc2cc(-c3cc(-c4nc(-c5ccc6ccccc6c5)nc(-c5cccc6sc7ccccc7c56)n4)c4c(c3)oc3ccccc34)ccc2c1 ; c1ccc2cc(-c3ccc4c(c3)oc3cccc(-c5nc(-c6ccc7ccccc7c6)nc(-c6cccc7sc8ccccc8c67)n5)c34)ccc2c1 ; c1ccc2cc(-c3nc(-c4cccc5sc6ccccc6c45)nc(-c4ccc(-c5ccc6ccccc6c5)c5oc6ccccc6c45)n3)ccc2c1]

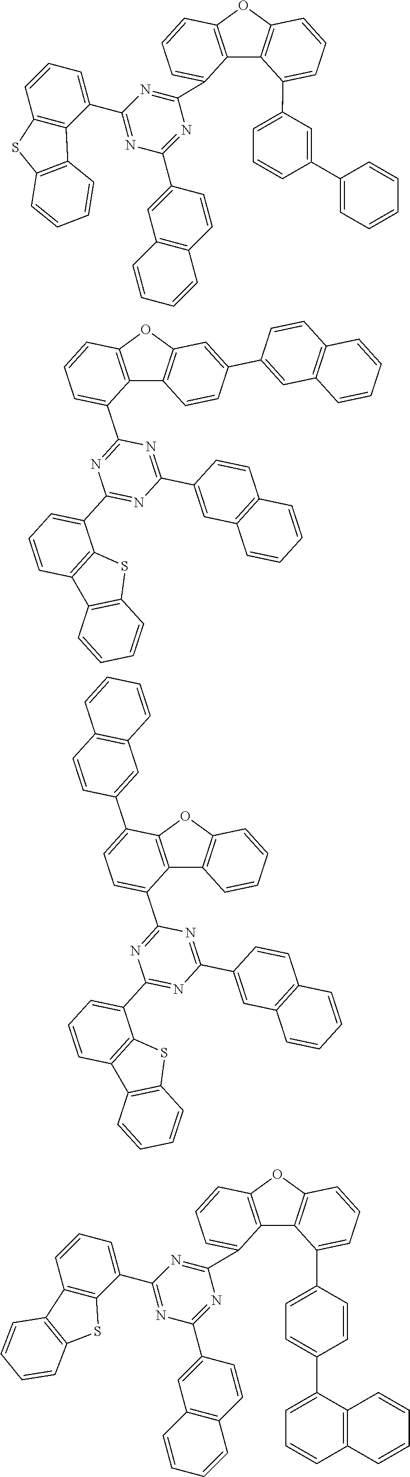

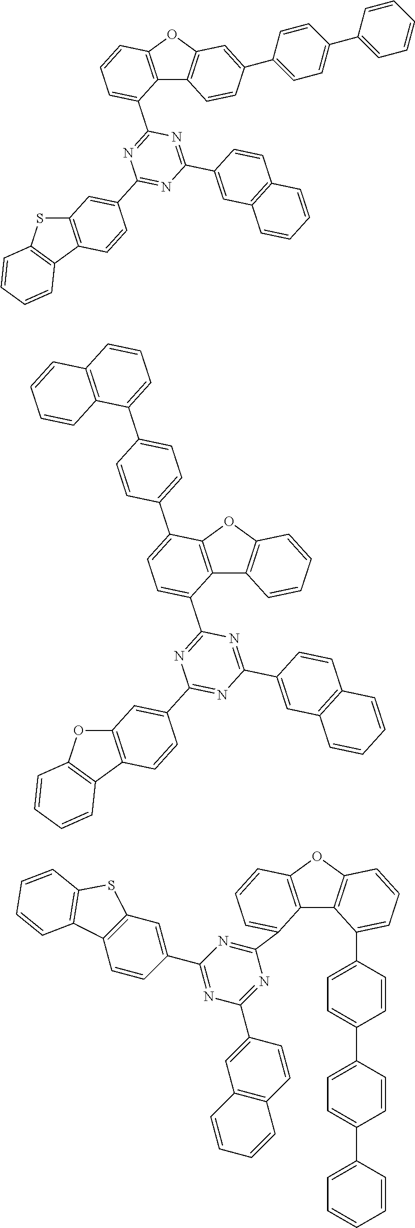

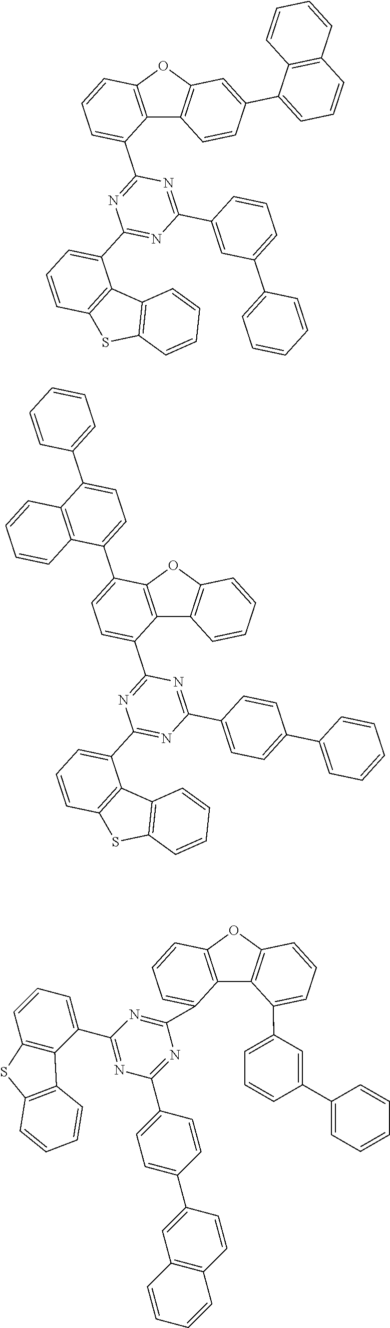 [SMILES: c1ccc(-c2cccc(-c3nc(-c4cccc5oc6cc(-c7cccc8ccccc78)ccc6c45)nc(-c4cccc5sc6ccccc6c45)n3)c2)cc1 ; c1ccc(-c2ccc(-c3nc(-c4cccc5sc6ccccc6c45)nc(-c4ccc(-c5ccc(-c6ccccc6)c6ccccc56)c5oc6ccccc6c45)n3)cc2)cc1 ; c1ccc(-c2cccc(-c3cccc4oc5cccc(-c6nc(-c7ccc(-c8ccc9ccccc9c8)cc7)nc(-c7cccc8sc9ccccc9c78)n6)c5c34)c2)cc1]

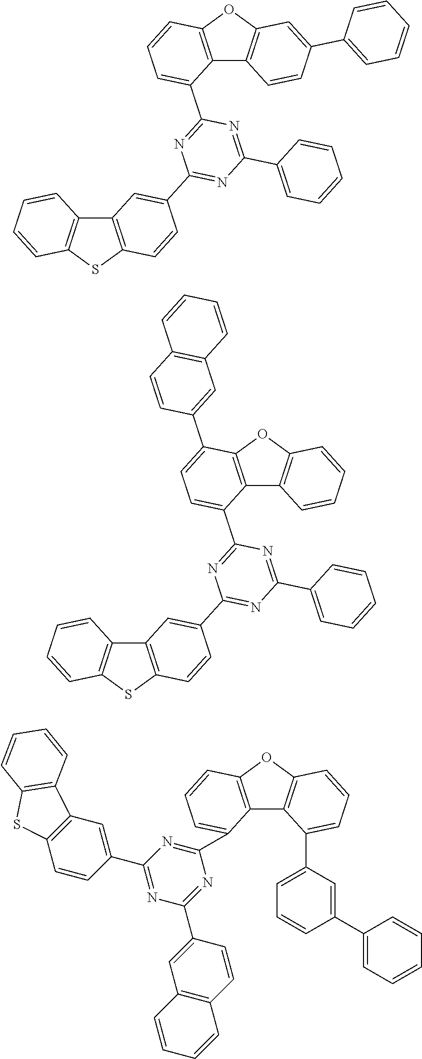 [SMILES: c1ccc(-c2ccc3c(c2)oc2cccc(-c4nc(-c5ccccc5)nc(-c5ccc6sc7ccccc7c6c5)n4)c23)cc1 ; c1ccc(-c2nc(-c3ccc4sc5ccccc5c4c3)nc(-c3ccc(-c4ccc5ccccc5c4)c4oc5ccccc5c34)n2)cc1 ; c1ccc(-c2cccc(-c3cccc4oc5cccc(-c6nc(-c7ccc8ccccc8c7)nc(-c7ccc8sc9ccccc9c8c7)n6)c5c34)c2)cc1]

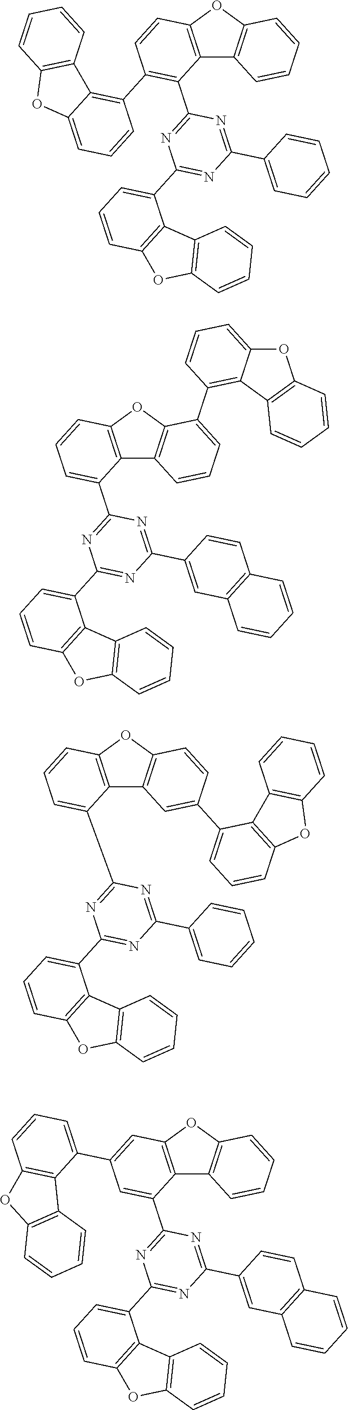

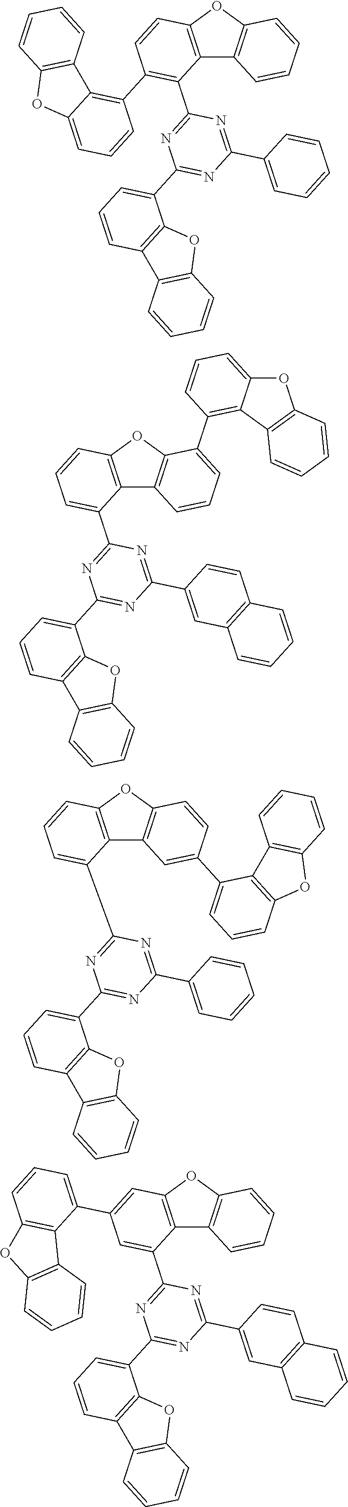

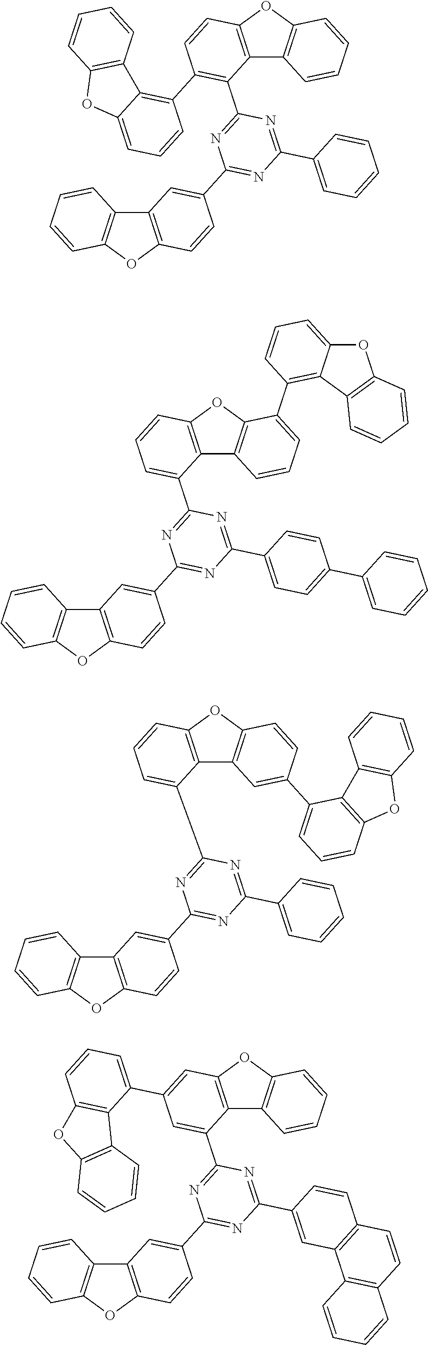

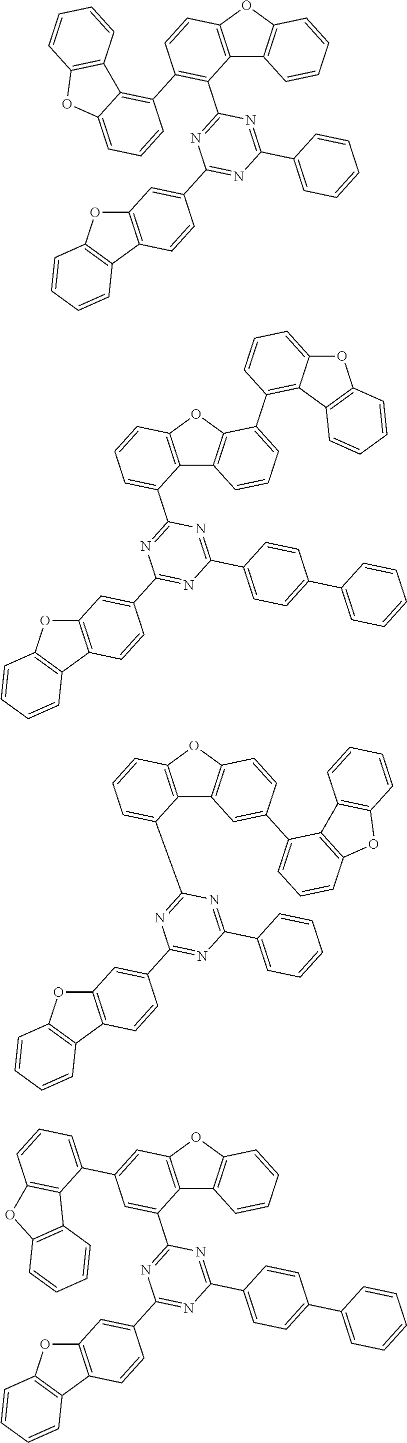

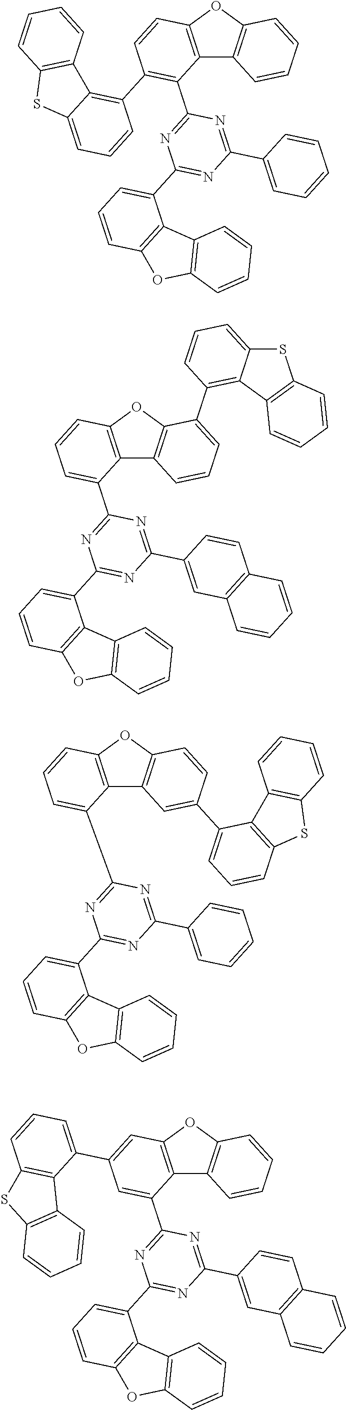

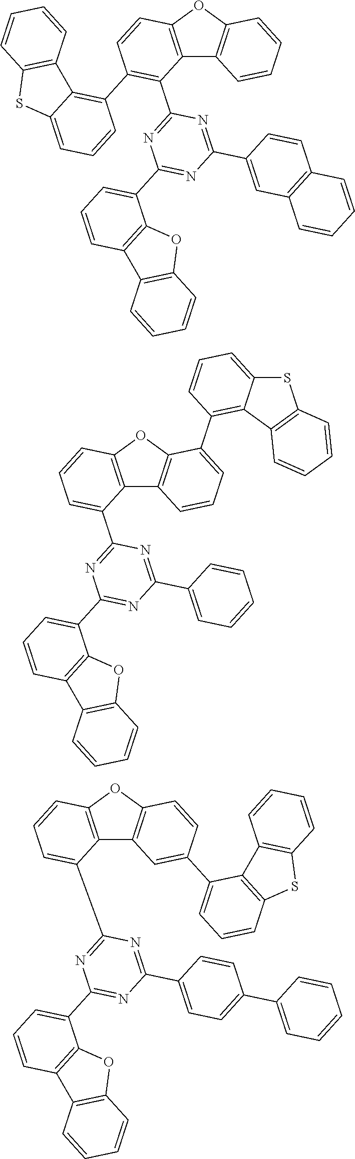

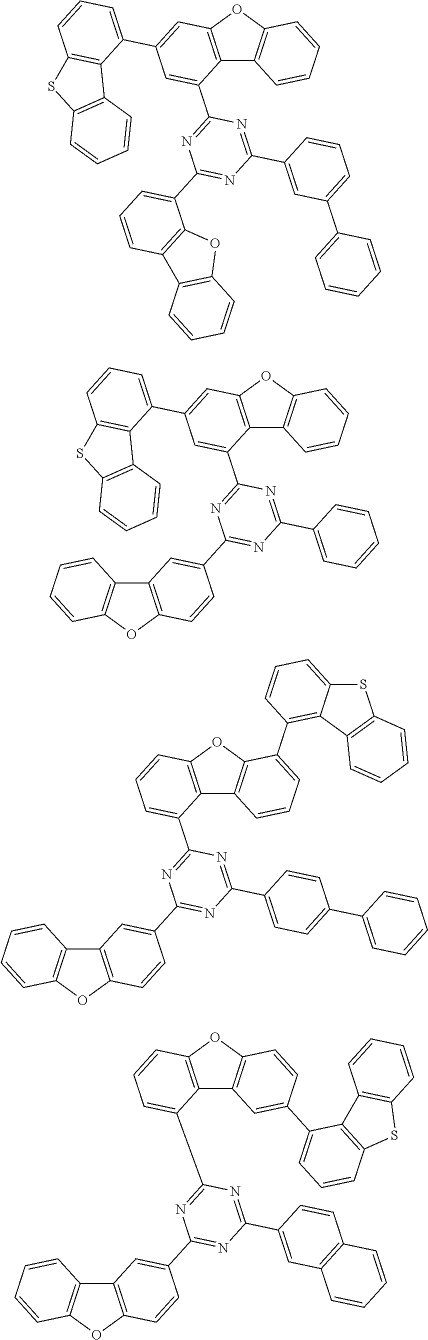 [SMILES: c1ccc(-c2cccc(-c3nc(-c4cccc5c4oc4ccccc45)nc(-c4cc(-c5cccc6sc7ccccc7c56)cc5oc6ccccc6c45)n3)c2)cc1 ; c1ccc(-c2nc(-c3ccc4oc5ccccc5c4c3)nc(-c3cc(-c4cccc5sc6ccccc6c45)cc4oc5ccccc5c34)n2)cc1 ; c1ccc(-c2ccc(-c3nc(-c4ccc5oc6ccccc6c5c4)nc(-c4cccc5oc6c(-c7cccc8sc9ccccc9c78)cccc6c45)n3)cc2)cc1 ; c1ccc2cc(-c3nc(-c4ccc5oc6ccccc6c5c4)nc(-c4cccc5oc6ccc(-c7cccc8sc9ccccc9c78)cc6c45)n3)ccc2c1]

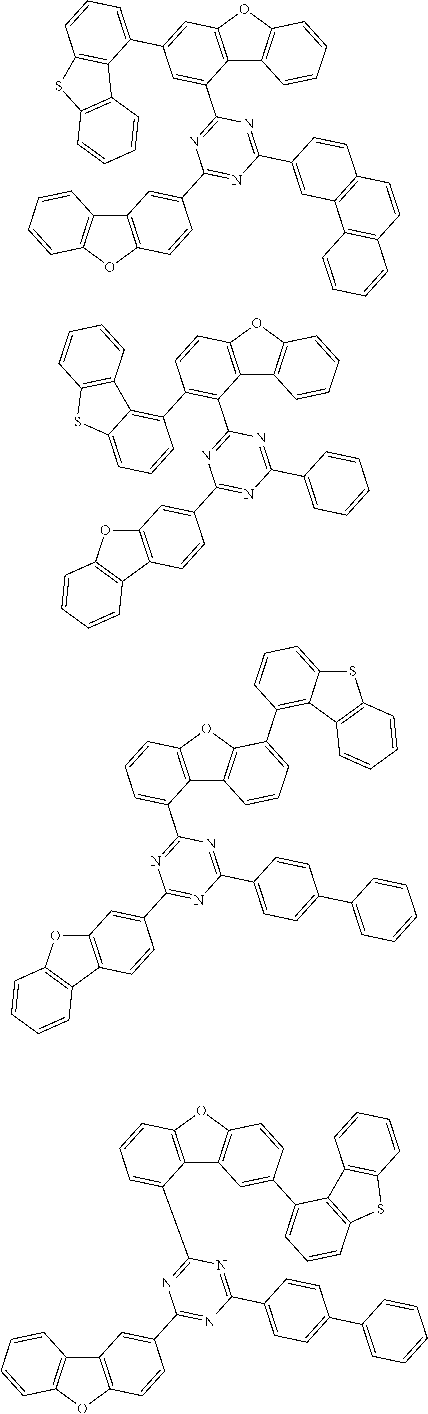 [SMILES: c1ccc2c(c1)ccc1ccc(-c3nc(-c4ccc5oc6ccccc6c5c4)nc(-c4cc(-c5cccc6sc7ccccc7c56)cc5oc6ccccc6c45)n3)cc12 ; c1ccc(-c2nc(-c3ccc4c(c3)oc3ccccc34)nc(-c3c(-c4cccc5sc6ccccc6c45)ccc4oc5ccccc5c34)n2)cc1 ; c1ccc(-c2ccc(-c3nc(-c4ccc5c(c4)oc4ccccc45)nc(-c4cccc5oc6c(-c7cccc8sc9ccccc9c78)cccc6c45)n3)cc2)cc1 ; c1ccc(-c2ccc(-c3nc(-c4ccc5oc6ccccc6c5c4)nc(-c4cccc5oc6ccc(-c7cccc8sc9ccccc9c78)cc6c45)n3)cc2)cc1]

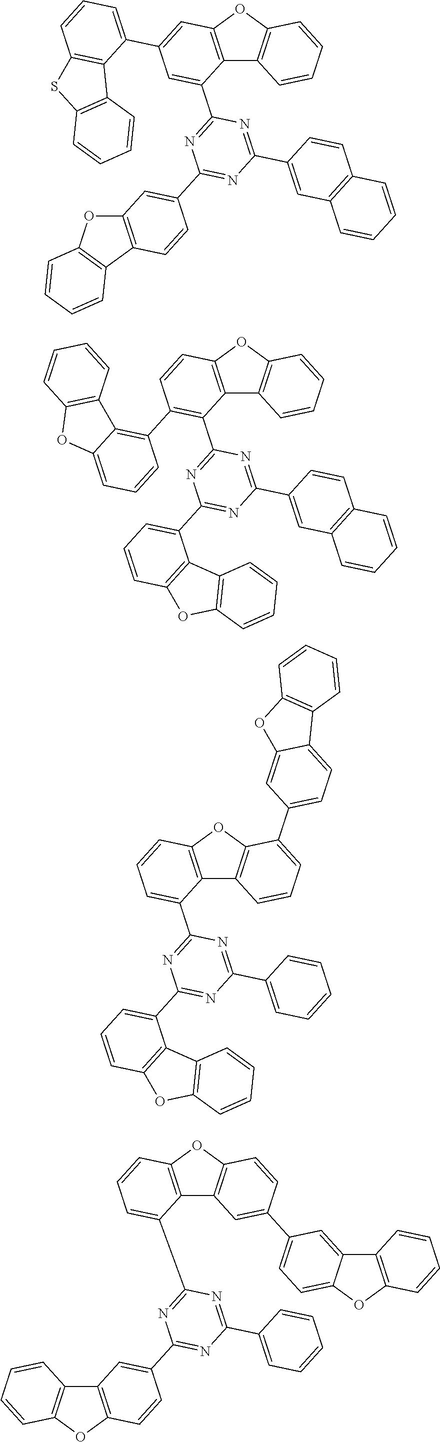

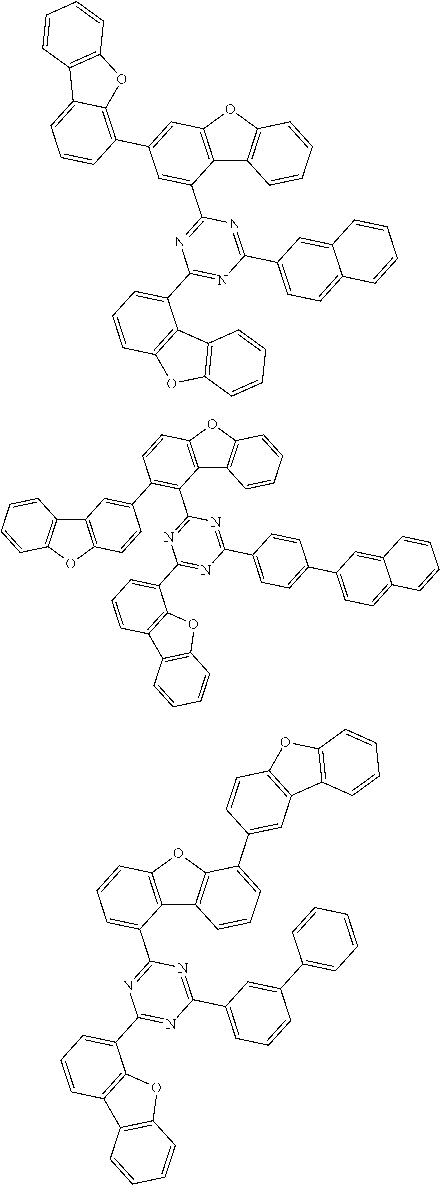

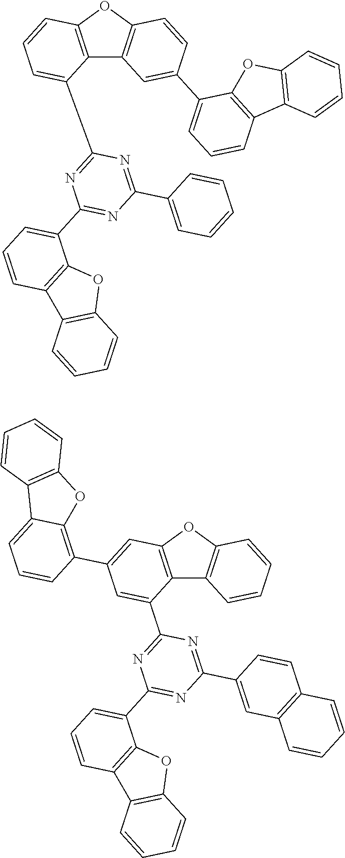 [SMILES: c1ccc(-c2nc(-c3cccc4c3oc3ccccc34)nc(-c3cccc4oc5ccc(-c6cccc7c6oc6ccccc67)cc5c34)n2)cc1 ; c1ccc2cc(-c3nc(-c4cccc5c4oc4ccccc45)nc(-c4cc(-c5cccc6c5oc5ccccc56)cc5oc6ccccc6c45)n3)ccc2c1]

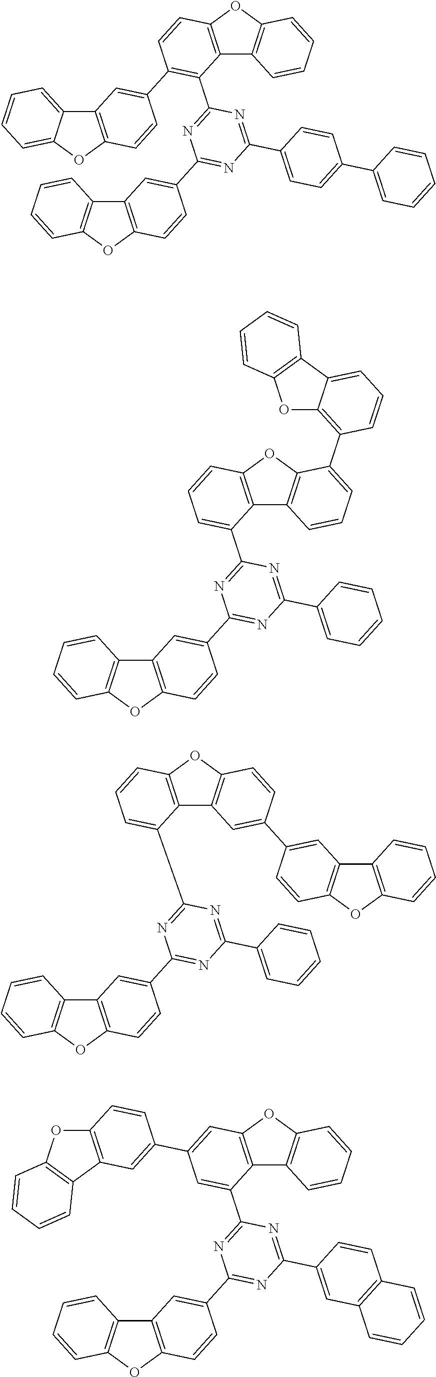 [SMILES: c1ccc(-c2ccc(-c3nc(-c4ccc5oc6ccccc6c5c4)nc(-c4c(-c5ccc6oc7ccccc7c6c5)ccc5oc6ccccc6c45)n3)cc2)cc1 ; c1ccc(-c2nc(-c3ccc4oc5ccccc5c4c3)nc(-c3cccc4oc5c(-c6cccc7c6oc6ccccc67)cccc5c34)n2)cc1 ; c1ccc(-c2nc(-c3ccc4oc5ccccc5c4c3)nc(-c3cccc4oc5ccc(-c6ccc7oc8ccccc8c7c6)cc5c34)n2)cc1 ; c1ccc2cc(-c3nc(-c4ccc5oc6ccccc6c5c4)nc(-c4cc(-c5ccc6oc7ccccc7c6c5)cc5oc6ccccc6c45)n3)ccc2c1]

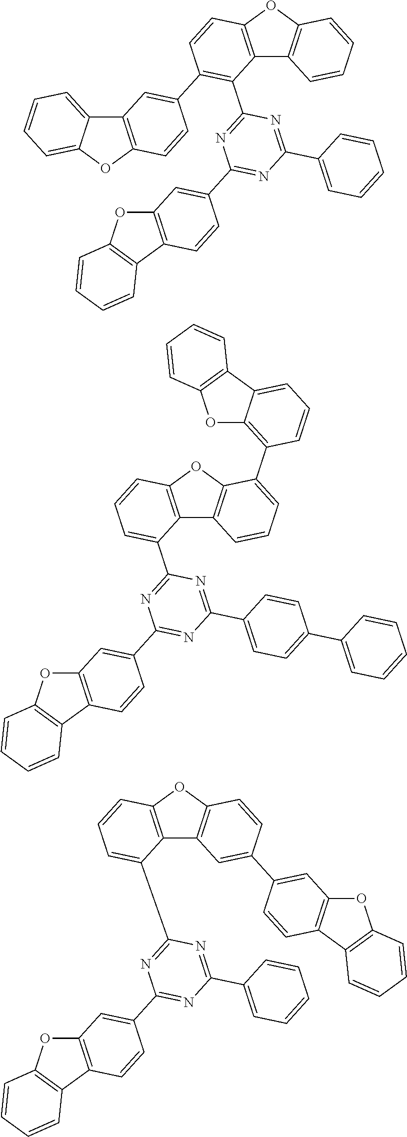 [SMILES: c1ccc(-c2nc(-c3ccc4c(c3)oc3ccccc34)nc(-c3c(-c4ccc5oc6ccccc6c5c4)ccc4oc5ccccc5c34)n2)cc1 ; c1ccc(-c2ccc(-c3nc(-c4ccc5c(c4)oc4ccccc45)nc(-c4cccc5oc6c(-c7cccc8c7oc7ccccc78)cccc6c45)n3)cc2)cc1 ; c1ccc(-c2nc(-c3ccc4c(c3)oc3ccccc34)nc(-c3cccc4oc5ccc(-c6ccc7c(c6)oc6ccccc67)cc5c34)n2)cc1]

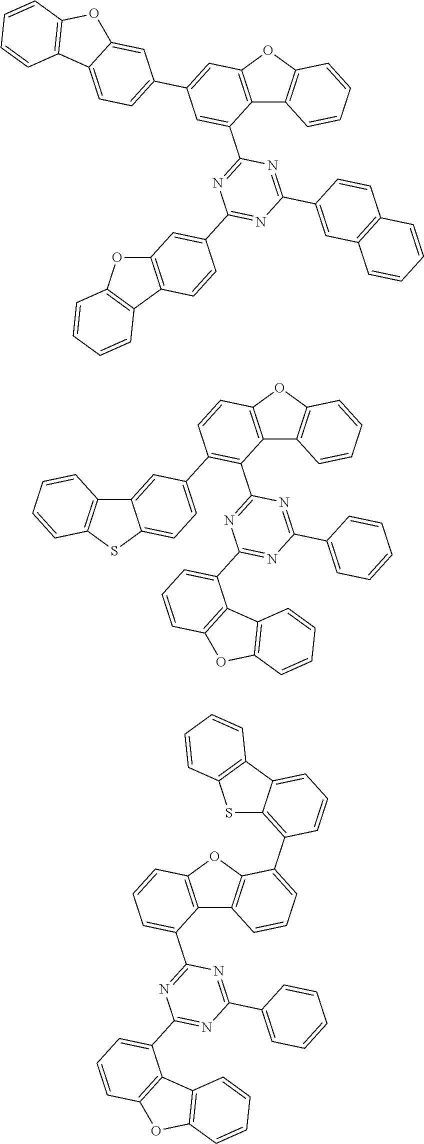 [SMILES: c1ccc2cc(-c3nc(-c4ccc5c(c4)oc4ccccc45)nc(-c4cc(-c5ccc6c(c5)oc5ccccc56)cc5oc6ccccc6c45)n3)ccc2c1 ; c1ccc(-c2nc(-c3cccc4oc5ccccc5c34)nc(-c3c(-c4ccc5sc6ccccc6c5c4)ccc4oc5ccccc5c34)n2)cc1 ; c1ccc(-c2nc(-c3cccc4oc5ccccc5c34)nc(-c3cccc4oc5c(-c6cccc7c6sc6ccccc67)cccc5c34)n2)cc1]

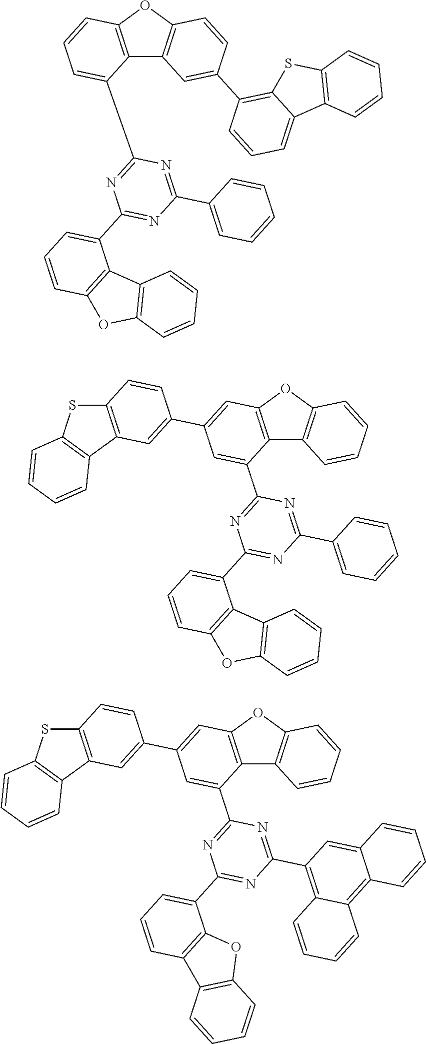 [SMILES: c1ccc(-c2nc(-c3cccc4oc5ccccc5c34)nc(-c3cccc4oc5ccc(-c6cccc7c6sc6ccccc67)cc5c34)n2)cc1 ; c1ccc(-c2nc(-c3cccc4oc5ccccc5c34)nc(-c3cc(-c4ccc5sc6ccccc6c5c4)cc4oc5ccccc5c34)n2)cc1 ; c1ccc2c(c1)cc(-c1nc(-c3cccc4c3oc3ccccc34)nc(-c3cc(-c4ccc5sc6ccccc6c5c4)cc4oc5ccccc5c34)n1)c1ccccc12]

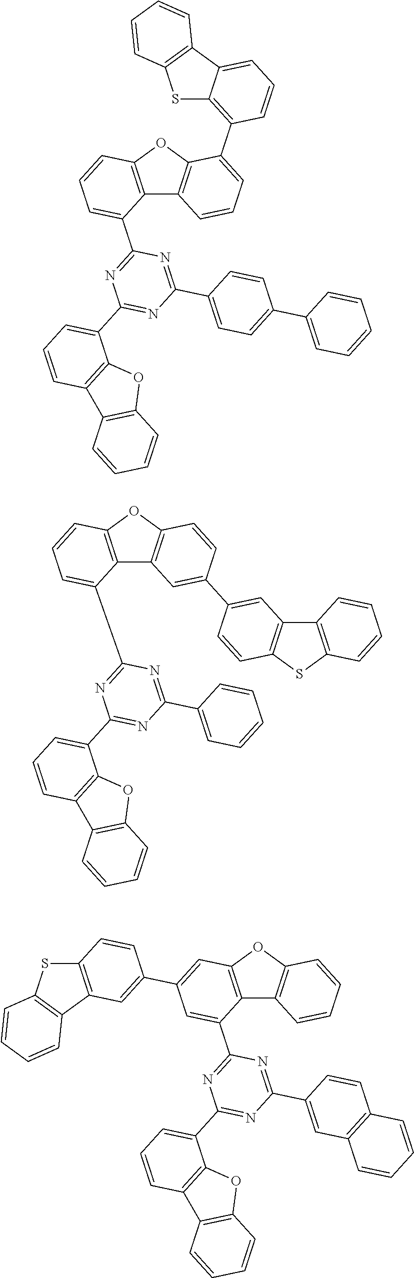

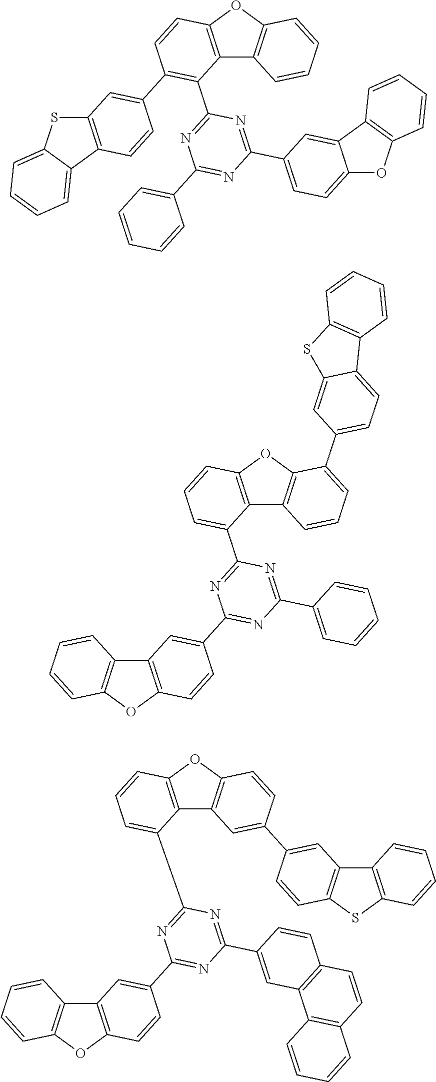 [SMILES: c1ccc(-c2nc(-c3ccc4oc5ccccc5c4c3)nc(-c3c(-c4ccc5c(c4)sc4ccccc45)ccc4oc5ccccc5c34)n2)cc1 ; c1ccc(-c2nc(-c3ccc4oc5ccccc5c4c3)nc(-c3cccc4oc5c(-c6ccc7c(c6)sc6ccccc67)cccc5c34)n2)cc1 ; c1ccc2c(c1)ccc1ccc(-c3nc(-c4ccc5oc6ccccc6c5c4)nc(-c4cccc5oc6ccc(-c7ccc8sc9ccccc9c8c7)cc6c45)n3)cc12]

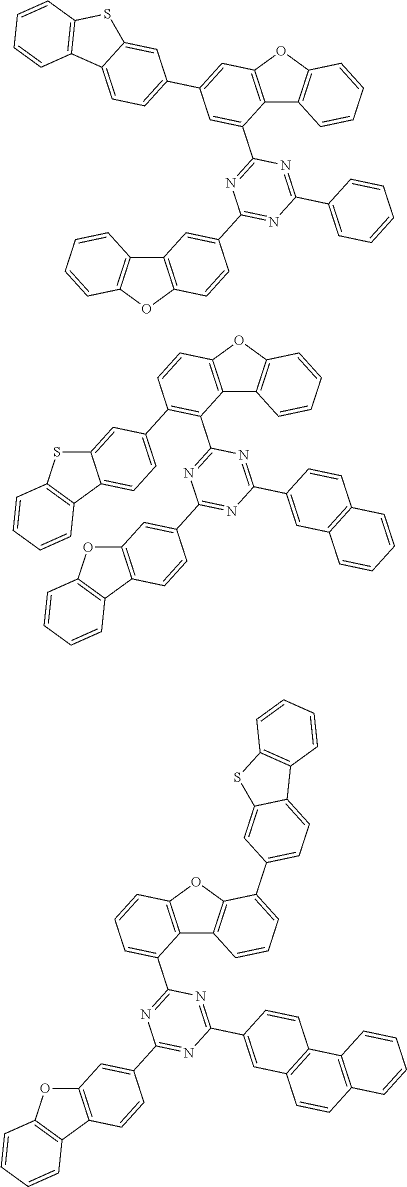 [SMILES: c1ccc(-c2nc(-c3ccc4oc5ccccc5c4c3)nc(-c3cc(-c4ccc5c(c4)sc4ccccc45)cc4oc5ccccc5c34)n2)cc1 ; c1ccc2cc(-c3nc(-c4ccc5c(c4)oc4ccccc45)nc(-c4c(-c5ccc6c(c5)sc5ccccc56)ccc5oc6ccccc6c45)n3)ccc2c1 ; c1ccc2c(c1)ccc1cc(-c3nc(-c4ccc5c(c4)oc4ccccc45)nc(-c4cccc5oc6c(-c7ccc8c(c7)sc7ccccc78)cccc6c45)n3)ccc12]

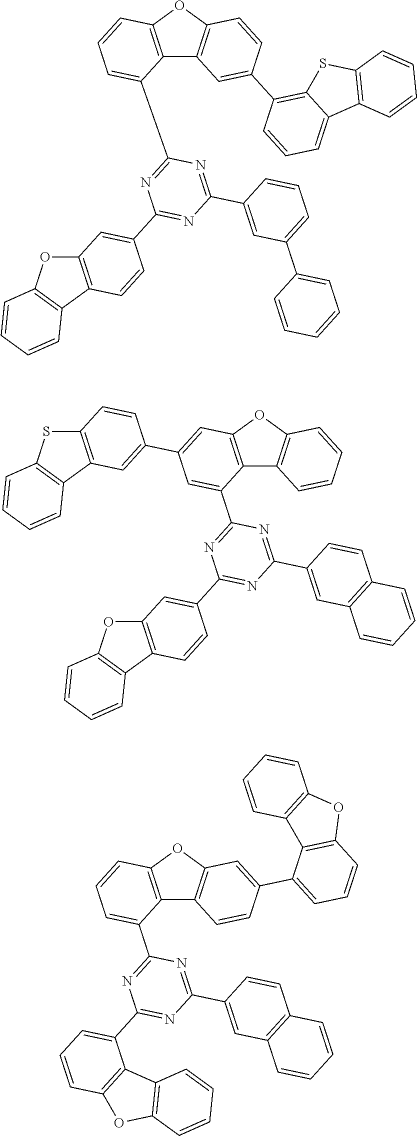 [SMILES: c1ccc(-c2cccc(-c3nc(-c4ccc5c(c4)oc4ccccc45)nc(-c4cccc5oc6ccc(-c7cccc8c7sc7ccccc78)cc6c45)n3)c2)cc1 ; c1ccc2cc(-c3nc(-c4ccc5c(c4)oc4ccccc45)nc(-c4cc(-c5ccc6sc7ccccc7c6c5)cc5oc6ccccc6c45)n3)ccc2c1 ; c1ccc2cc(-c3nc(-c4cccc5oc6ccccc6c45)nc(-c4cccc5oc6cc(-c7cccc8oc9ccccc9c78)ccc6c45)n3)ccc2c1]

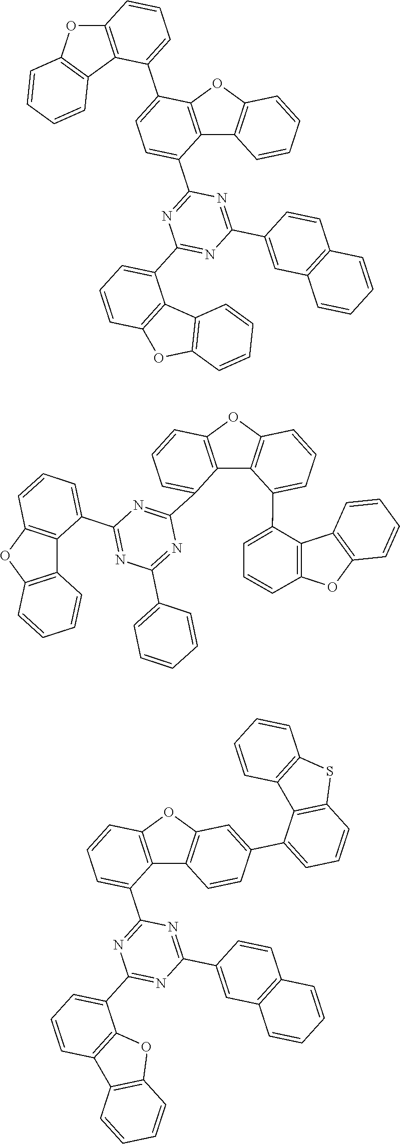 [SMILES: c1ccc2cc(-c3nc(-c4cccc5oc6ccccc6c45)nc(-c4ccc(-c5cccc6oc7ccccc7c56)c5oc6ccccc6c45)n3)ccc2c1 ; c1ccc(-c2nc(-c3cccc4oc5ccccc5c34)nc(-c3cccc4oc5cccc(-c6cccc7oc8ccccc8c67)c5c34)n2)cc1 ; c1ccc2cc(-c3nc(-c4cccc5c4oc4ccccc45)nc(-c4cccc5oc6cc(-c7cccc8sc9ccccc9c78)ccc6c45)n3)ccc2c1]

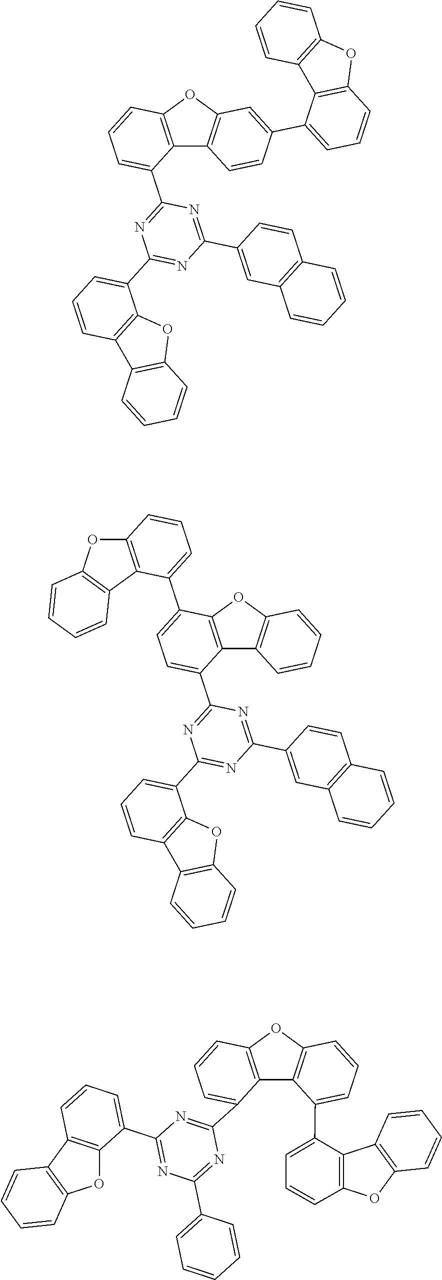 [SMILES: c1ccc2cc(-c3nc(-c4cccc5c4oc4ccccc45)nc(-c4cccc5oc6cc(-c7cccc8oc9ccccc9c78)ccc6c45)n3)ccc2c1 ; c1ccc2cc(-c3nc(-c4cccc5c4oc4ccccc45)nc(-c4ccc(-c5cccc6oc7ccccc7c56)c5oc6ccccc6c45)n3)ccc2c1 ; c1ccc(-c2nc(-c3cccc4c3oc3ccccc34)nc(-c3cccc4oc5cccc(-c6cccc7oc8ccccc8c67)c5c34)n2)cc1]

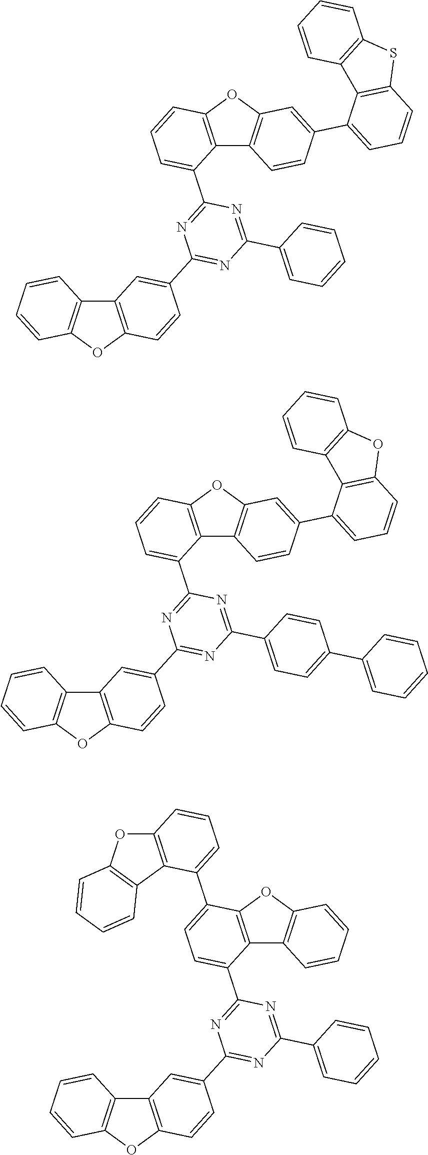 [SMILES: c1ccc(-c2nc(-c3ccc4oc5ccccc5c4c3)nc(-c3cccc4oc5cc(-c6cccc7sc8ccccc8c67)ccc5c34)n2)cc1 ; c1ccc(-c2ccc(-c3nc(-c4ccc5oc6ccccc6c5c4)nc(-c4cccc5oc6cc(-c7cccc8oc9ccccc9c78)ccc6c45)n3)cc2)cc1 ; c1ccc(-c2nc(-c3ccc4oc5ccccc5c4c3)nc(-c3ccc(-c4cccc5oc6ccccc6c45)c4oc5ccccc5c34)n2)cc1]

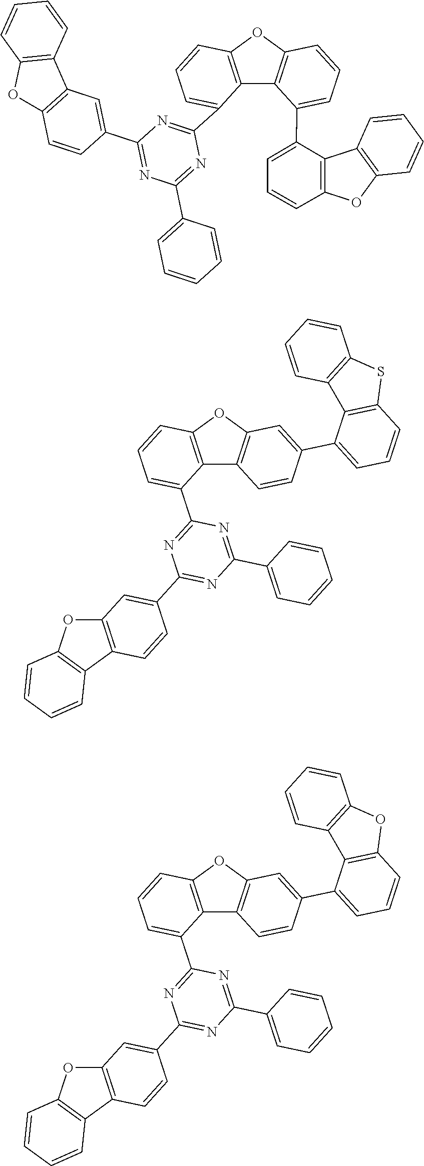 [SMILES: c1ccc(-c2nc(-c3ccc4oc5ccccc5c4c3)nc(-c3cccc4oc5cccc(-c6cccc7oc8ccccc8c67)c5c34)n2)cc1 ; c1ccc(-c2nc(-c3ccc4c(c3)oc3ccccc34)nc(-c3cccc4oc5cc(-c6cccc7sc8ccccc8c67)ccc5c34)n2)cc1 ; c1ccc(-c2nc(-c3ccc4c(c3)oc3ccccc34)nc(-c3cccc4oc5cc(-c6cccc7oc8ccccc8c67)ccc5c34)n2)cc1]

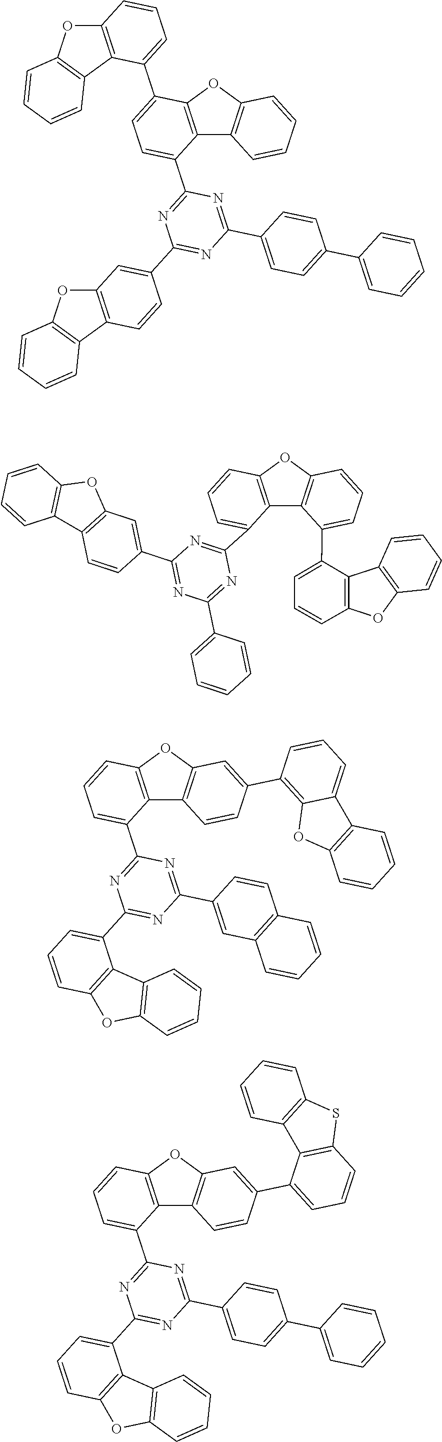 [SMILES: c1ccc(-c2ccc(-c3nc(-c4ccc5c(c4)oc4ccccc45)nc(-c4ccc(-c5cccc6oc7ccccc7c56)c5oc6ccccc6c45)n3)cc2)cc1 ; c1ccc(-c2nc(-c3ccc4c(c3)oc3ccccc34)nc(-c3cccc4oc5cccc(-c6cccc7oc8ccccc8c67)c5c34)n2)cc1 ; c1ccc2cc(-c3nc(-c4cccc5oc6ccccc6c45)nc(-c4cccc5oc6cc(-c7cccc8c7oc7ccccc78)ccc6c45)n3)ccc2c1 ; c1ccc(-c2ccc(-c3nc(-c4cccc5oc6ccccc6c45)nc(-c4cccc5oc6cc(-c7cccc8sc9ccccc9c78)ccc6c45)n3)cc2)cc1]

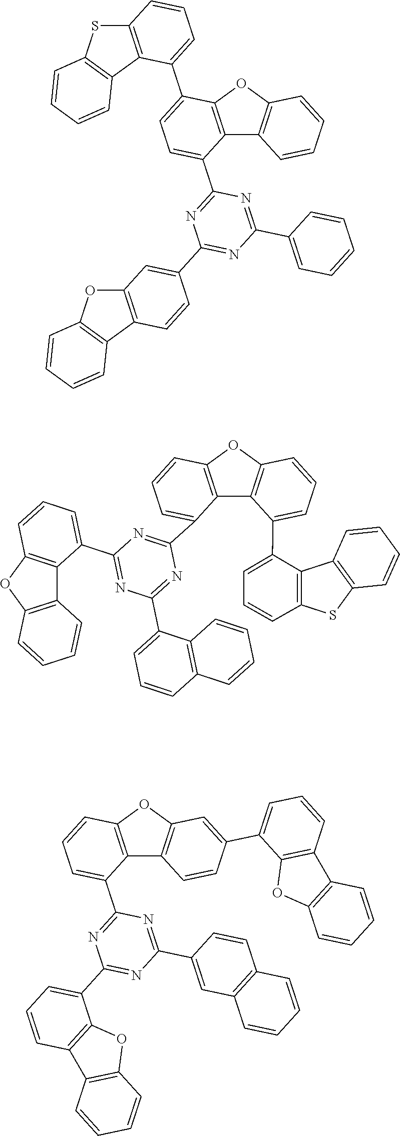 [SMILES: c1ccc(-c2nc(-c3ccc4c(c3)oc3ccccc34)nc(-c3ccc(-c4cccc5sc6ccccc6c45)c4oc5ccccc5c34)n2)cc1 ; c1ccc2c(-c3nc(-c4cccc5oc6ccccc6c45)nc(-c4cccc5oc6cccc(-c7cccc8sc9ccccc9c78)c6c45)n3)cccc2c1 ; c1ccc2cc(-c3nc(-c4cccc5c4oc4ccccc45)nc(-c4cccc5oc6cc(-c7cccc8c7oc7ccccc78)ccc6c45)n3)ccc2c1]

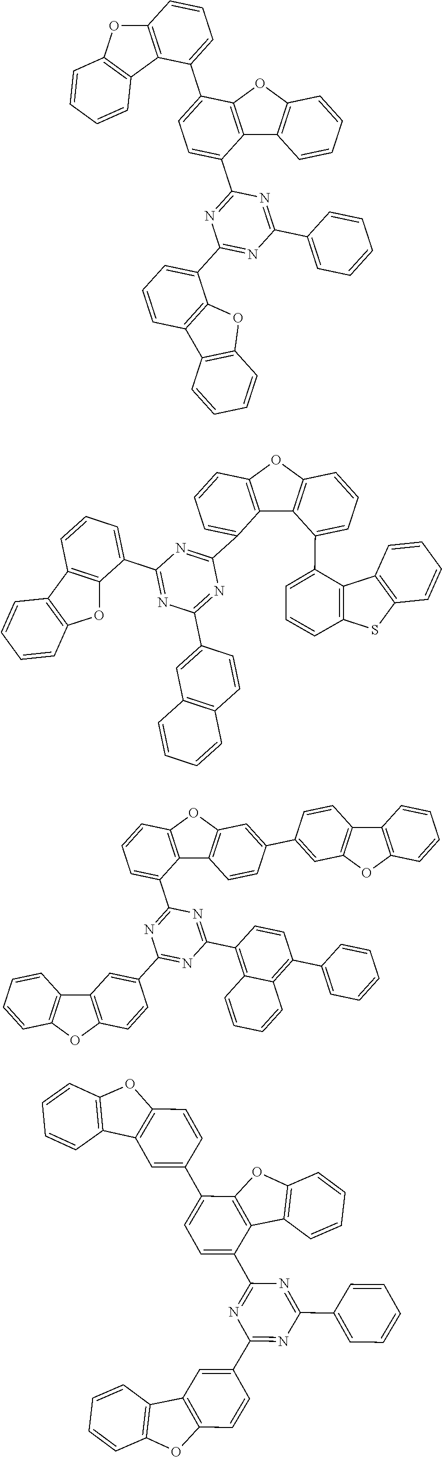

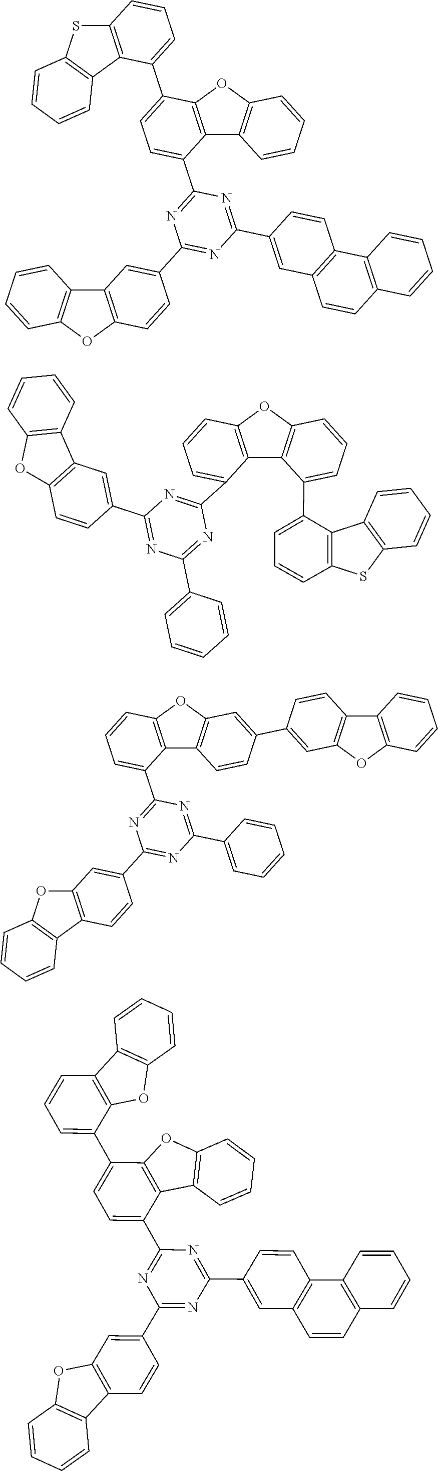 [SMILES: c1ccc2c(c1)ccc1cc(-c3nc(-c4ccc5oc6ccccc6c5c4)nc(-c4ccc(-c5cccc6sc7ccccc7c56)c5oc6ccccc6c45)n3)ccc12 ; c1ccc(-c2nc(-c3ccc4oc5ccccc5c4c3)nc(-c3cccc4oc5cccc(-c6cccc7sc8ccccc8c67)c5c34)n2)cc1 ; c1ccc(-c2nc(-c3ccc4c(c3)oc3ccccc34)nc(-c3cccc4oc5cc(-c6ccc7c(c6)oc6ccccc67)ccc5c34)n2)cc1 ; c1ccc2c(c1)ccc1cc(-c3nc(-c4ccc5c(c4)oc4ccccc45)nc(-c4ccc(-c5cccc6c5oc5ccccc56)c5oc6ccccc6c45)n3)ccc12]

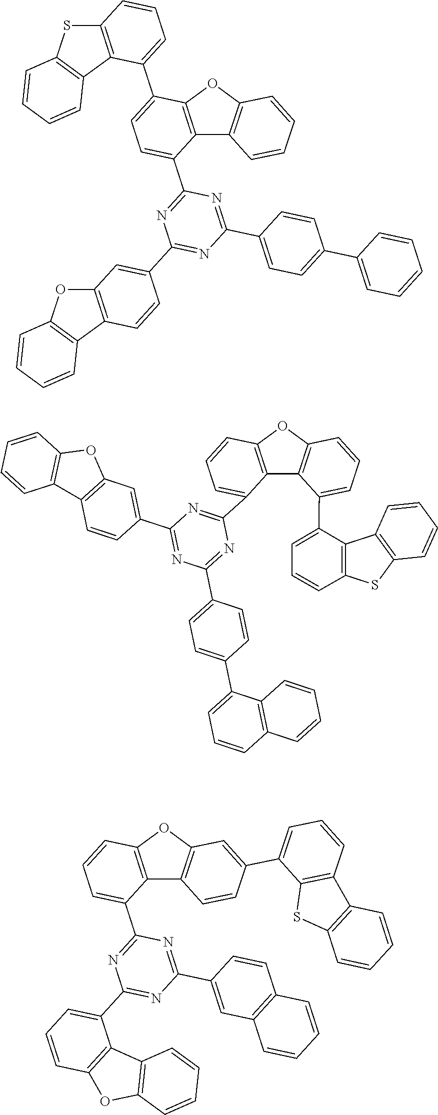

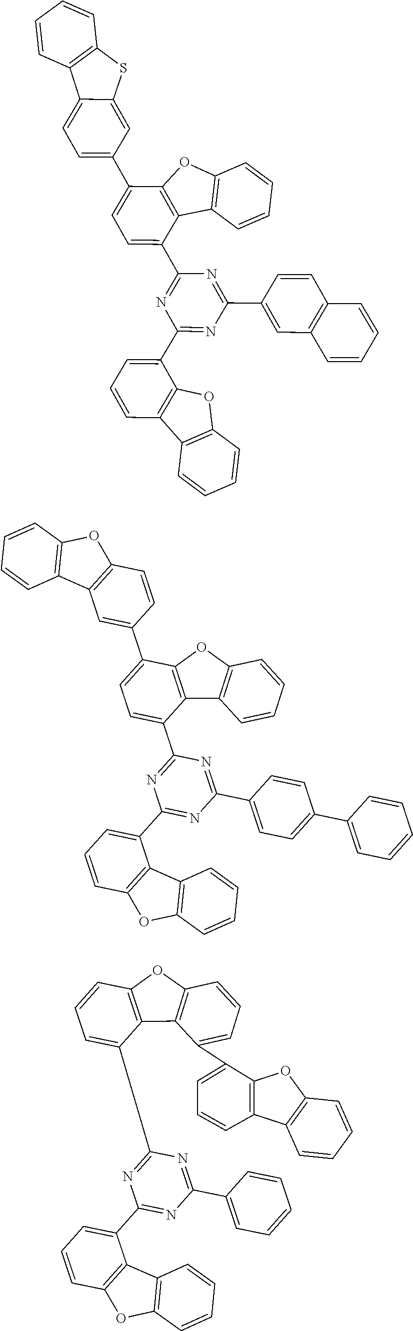 [SMILES: c1ccc2cc(-c3nc(-c4cccc5c4oc4ccccc45)nc(-c4ccc(-c5ccc6c(c5)sc5ccccc56)c5oc6ccccc6c45)n3)ccc2c1 ; c1ccc(-c2ccc(-c3nc(-c4cccc5oc6ccccc6c45)nc(-c4ccc(-c5ccc6oc7ccccc7c6c5)c5oc6ccccc6c45)n3)cc2)cc1 ; c1ccc(-c2nc(-c3cccc4oc5ccccc5c34)nc(-c3cccc4oc5cccc(-c6cccc7c6oc6ccccc67)c5c34)n2)cc1]

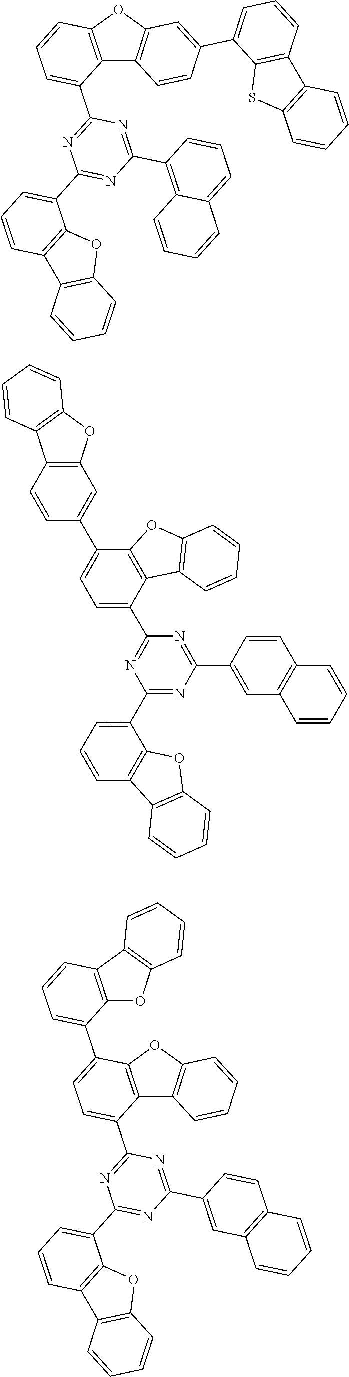

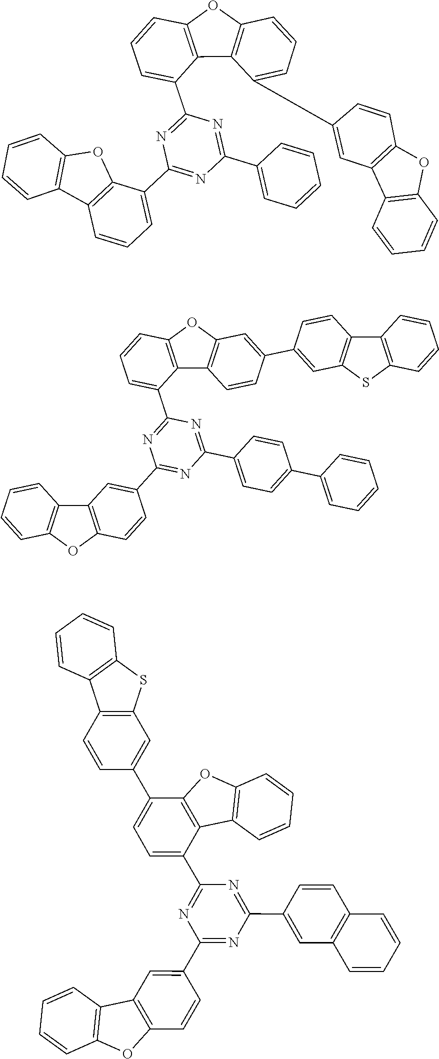 [SMILES: c1ccc(-c2nc(-c3cccc4c3oc3ccccc34)nc(-c3cccc4oc5cccc(-c6ccc7oc8ccccc8c7c6)c5c34)n2)cc1 ; c1ccc(-c2ccc(-c3nc(-c4ccc5oc6ccccc6c5c4)nc(-c4cccc5oc6cc(-c7ccc8c(c7)sc7ccccc78)ccc6c45)n3)cc2)cc1 ; c1ccc2cc(-c3nc(-c4ccc5oc6ccccc6c5c4)nc(-c4ccc(-c5ccc6c(c5)sc5ccccc56)c5oc6ccccc6c45)n3)ccc2c1]

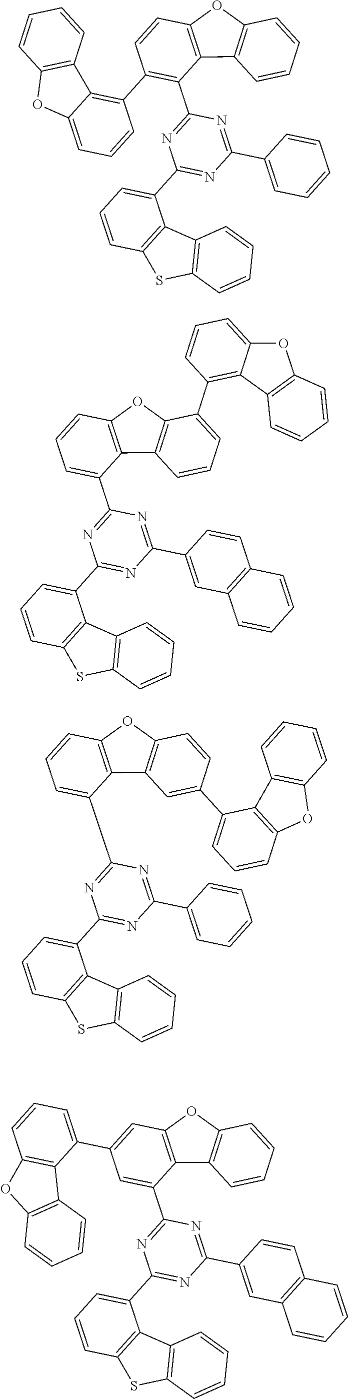

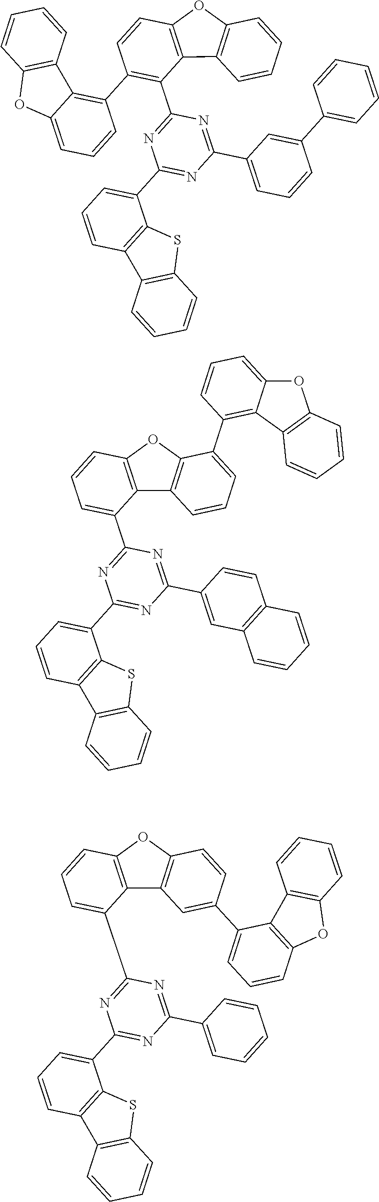

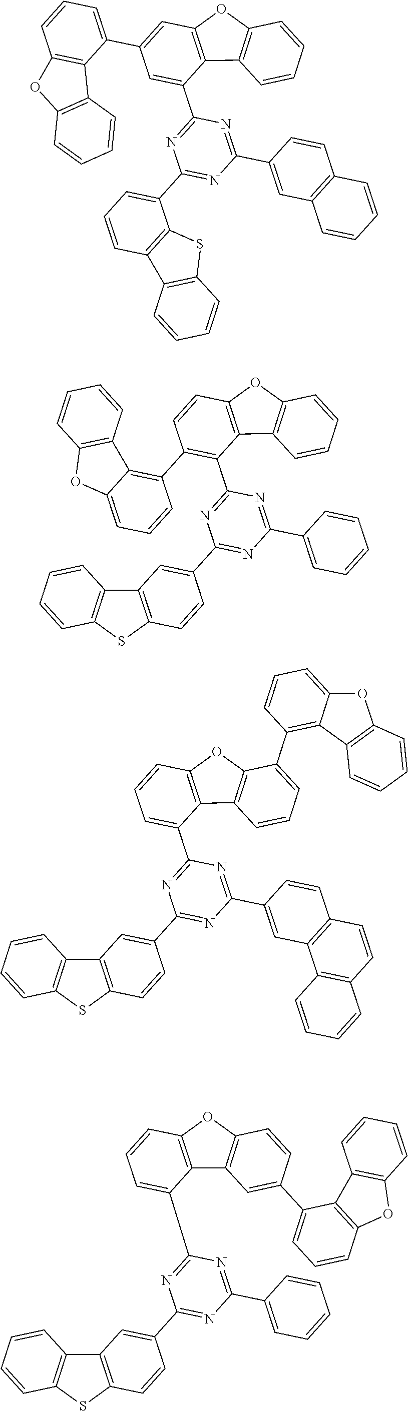 [SMILES: c1ccc2cc(-c3nc(-c4cccc5c4sc4ccccc45)nc(-c4cc(-c5cccc6oc7ccccc7c56)cc5oc6ccccc6c45)n3)ccc2c1 ; c1ccc(-c2nc(-c3ccc4sc5ccccc5c4c3)nc(-c3c(-c4cccc5oc6ccccc6c45)ccc4oc5ccccc5c34)n2)cc1 ; c1ccc2c(c1)ccc1ccc(-c3nc(-c4ccc5sc6ccccc6c5c4)nc(-c4cccc5oc6c(-c7cccc8oc9ccccc9c78)cccc6c45)n3)cc12 ; c1ccc(-c2nc(-c3ccc4sc5ccccc5c4c3)nc(-c3cccc4oc5ccc(-c6cccc7oc8ccccc8c67)cc5c34)n2)cc1]

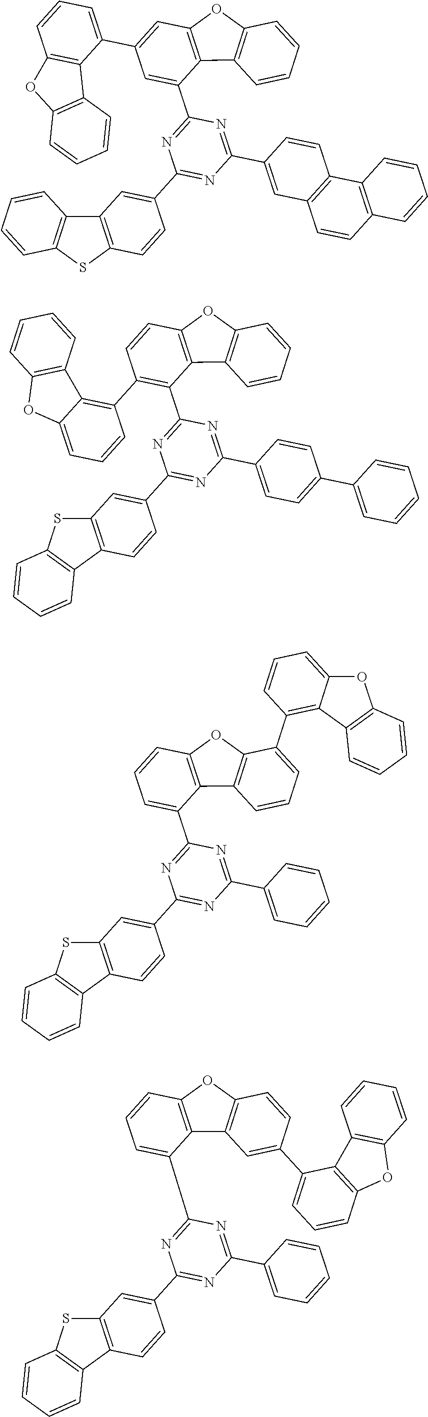 [SMILES: c1ccc2c(c1)ccc1cc(-c3nc(-c4ccc5sc6ccccc6c5c4)nc(-c4cc(-c5cccc6oc7ccccc7c56)cc5oc6ccccc6c45)n3)ccc12 ; c1ccc(-c2ccc(-c3nc(-c4ccc5c(c4)sc4ccccc45)nc(-c4c(-c5cccc6oc7ccccc7c56)ccc5oc6ccccc6c45)n3)cc2)cc1 ; c1ccc(-c2nc(-c3ccc4c(c3)sc3ccccc34)nc(-c3cccc4oc5c(-c6cccc7oc8ccccc8c67)cccc5c34)n2)cc1 ; c1ccc(-c2nc(-c3ccc4c(c3)sc3ccccc34)nc(-c3cccc4oc5ccc(-c6cccc7oc8ccccc8c67)cc5c34)n2)cc1]

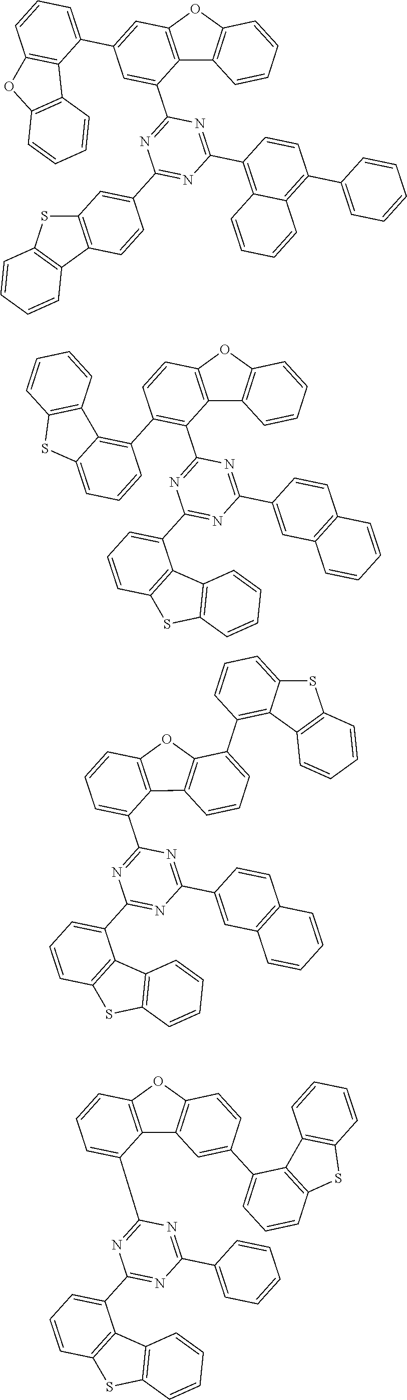

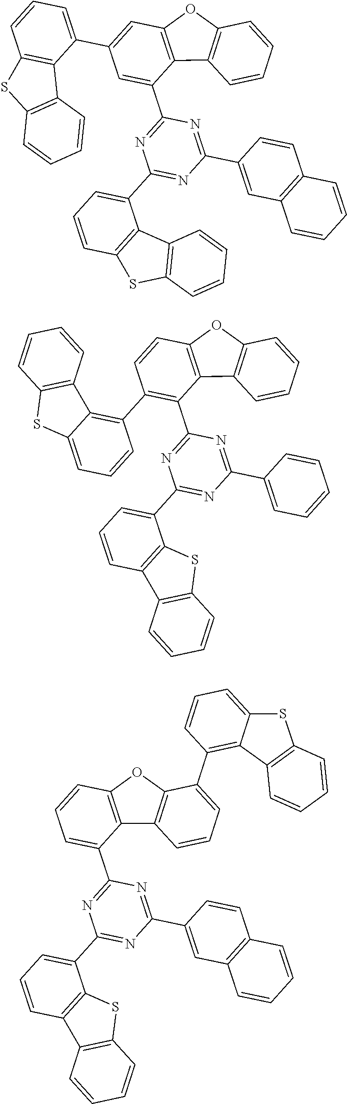

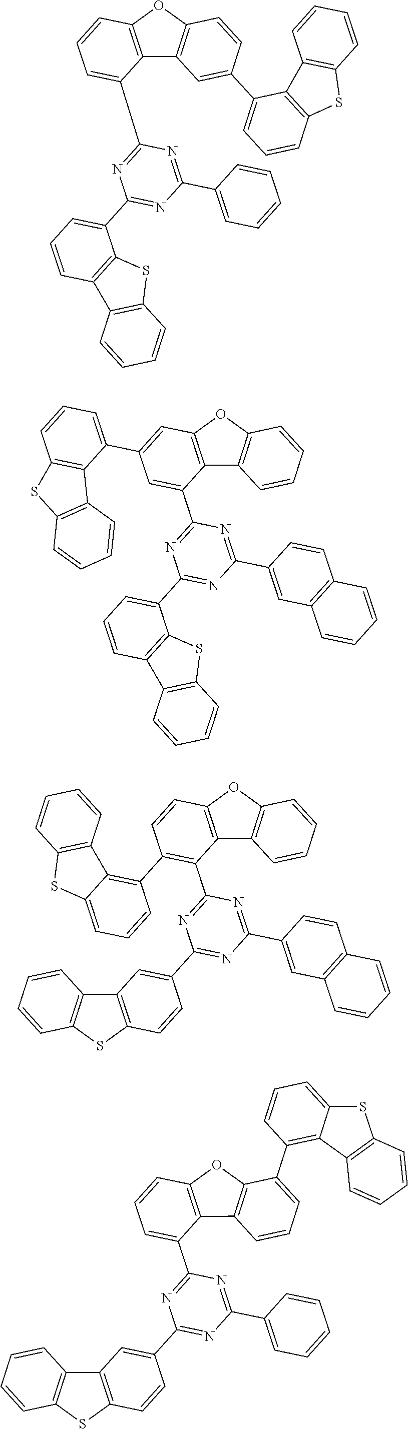 [SMILES: c1ccc(-c2nc(-c3cccc4c3sc3ccccc34)nc(-c3cccc4oc5ccc(-c6cccc7sc8ccccc8c67)cc5c34)n2)cc1 ; c1ccc2cc(-c3nc(-c4cccc5c4sc4ccccc45)nc(-c4cc(-c5cccc6sc7ccccc7c56)cc5oc6ccccc6c45)n3)ccc2c1 ; c1ccc2cc(-c3nc(-c4ccc5sc6ccccc6c5c4)nc(-c4c(-c5cccc6sc7ccccc7c56)ccc5oc6ccccc6c45)n3)ccc2c1 ; c1ccc(-c2nc(-c3ccc4sc5ccccc5c4c3)nc(-c3cccc4oc5c(-c6cccc7sc8ccccc8c67)cccc5c34)n2)cc1]

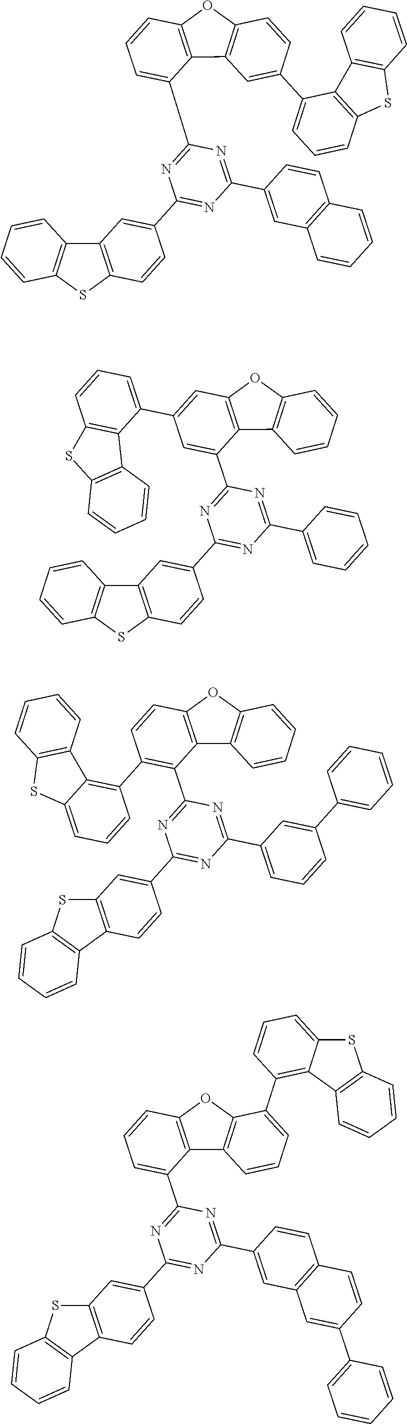 [SMILES: c1ccc2cc(-c3nc(-c4ccc5sc6ccccc6c5c4)nc(-c4cccc5oc6ccc(-c7cccc8sc9ccccc9c78)cc6c45)n3)ccc2c1 ; c1ccc(-c2nc(-c3ccc4sc5ccccc5c4c3)nc(-c3cc(-c4cccc5sc6ccccc6c45)cc4oc5ccccc5c34)n2)cc1 ; c1ccc(-c2cccc(-c3nc(-c4ccc5c(c4)sc4ccccc45)nc(-c4c(-c5cccc6sc7ccccc7c56)ccc5oc6ccccc6c45)n3)c2)cc1 ; c1ccc(-c2ccc3ccc(-c4nc(-c5ccc6c(c5)sc5ccccc56)nc(-c5cccc6oc7c(-c8cccc9sc%10ccccc%10c89)cccc7c56)n4)cc3c2)cc1]

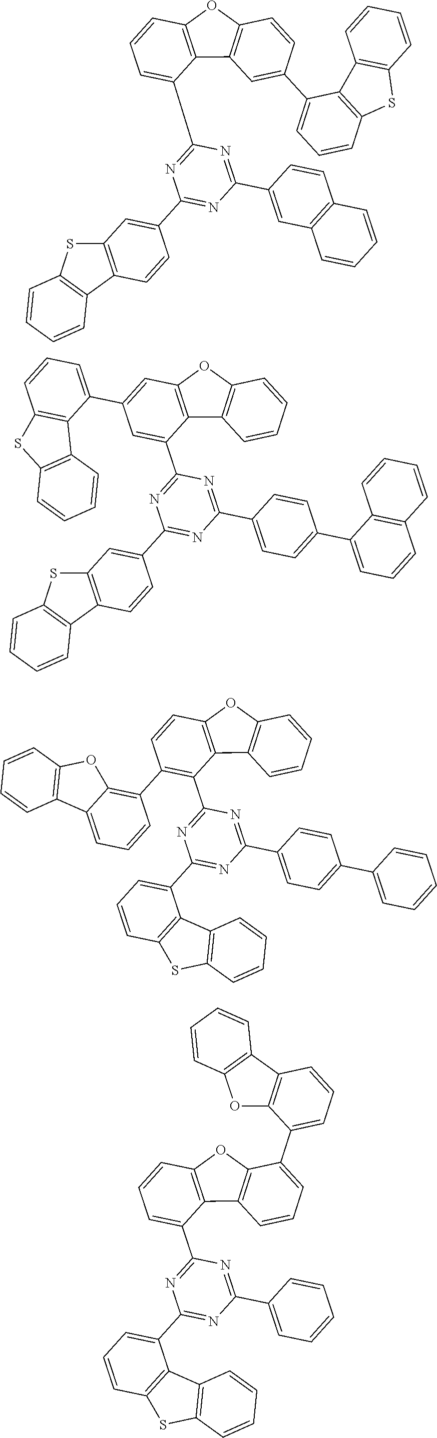

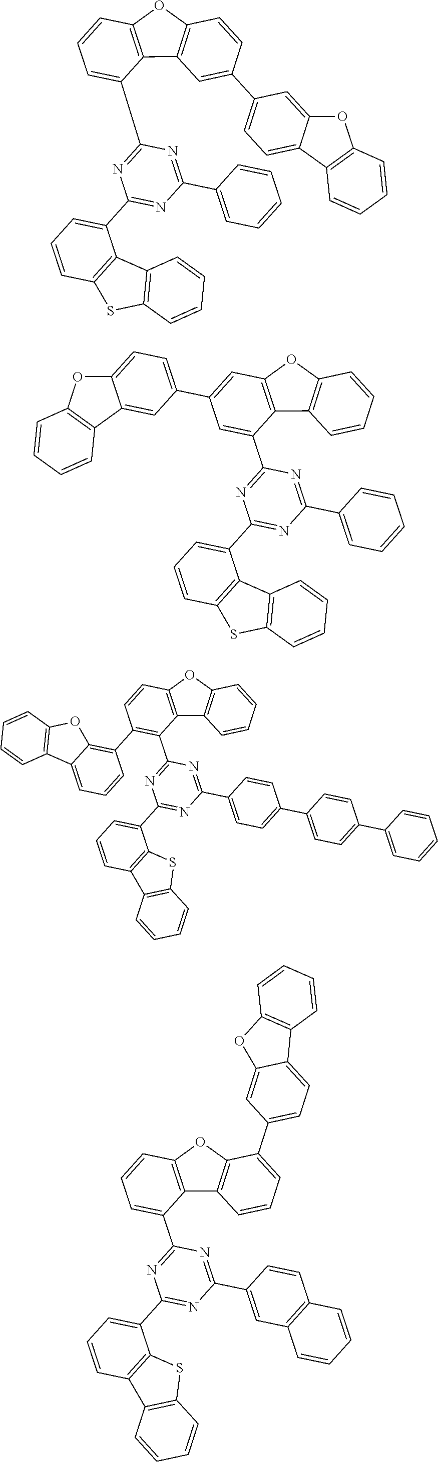

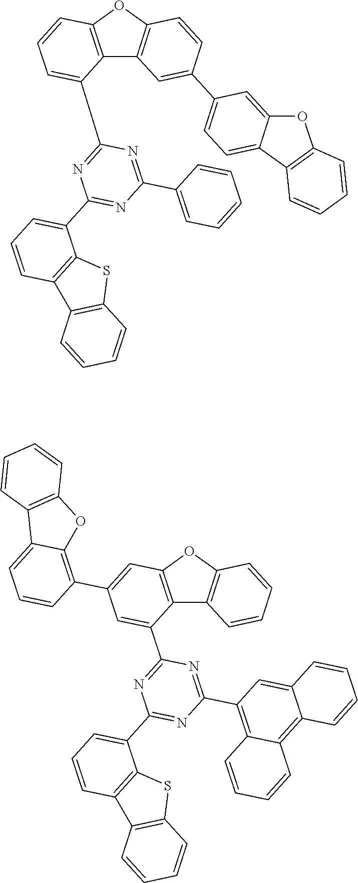 [SMILES: c1ccc(-c2nc(-c3cccc4c3sc3ccccc34)nc(-c3cccc4oc5ccc(-c6ccc7c(c6)oc6ccccc67)cc5c34)n2)cc1 ; c1ccc2c(c1)cc(-c1nc(-c3cccc4c3sc3ccccc34)nc(-c3cc(-c4cccc5c4oc4ccccc45)cc4oc5ccccc5c34)n1)c1ccccc12]

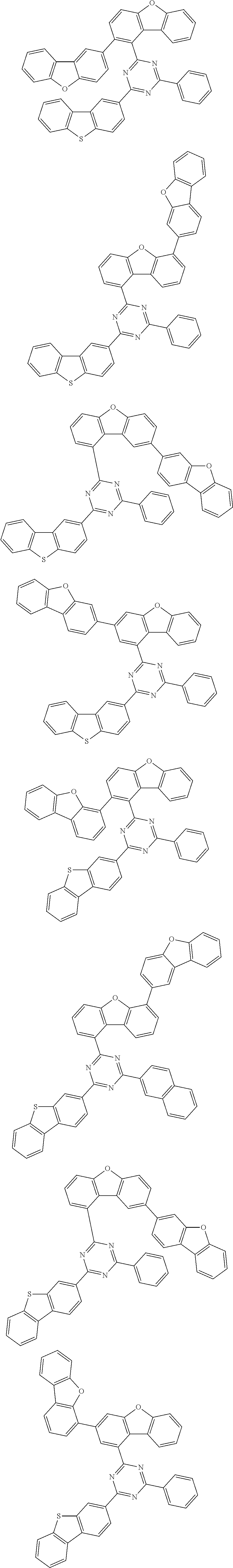

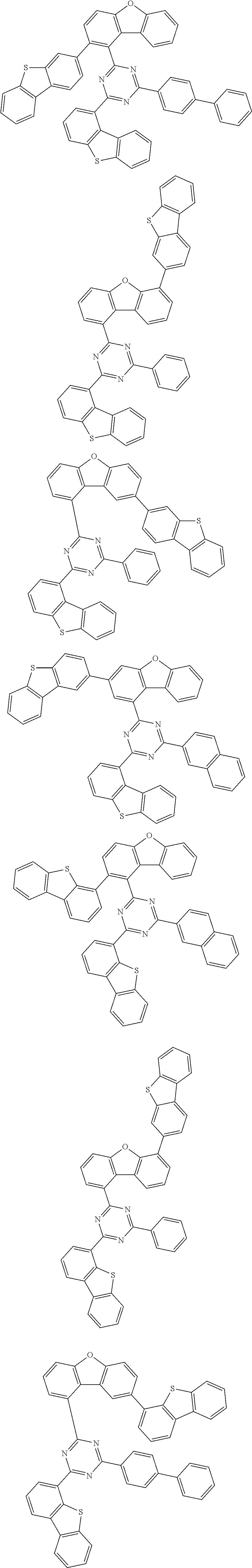

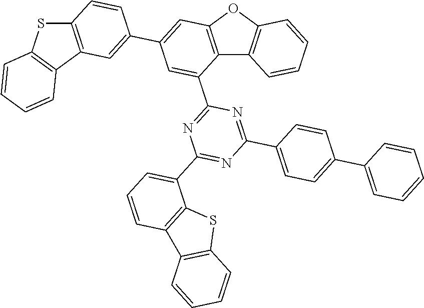 [SMILES: c1ccc(-c2ccc(-c3nc(-c4cccc5c4sc4ccccc45)nc(-c4cc(-c5ccc6sc7ccccc7c6c5)cc5oc6ccccc6c45)n3)cc2)cc1]

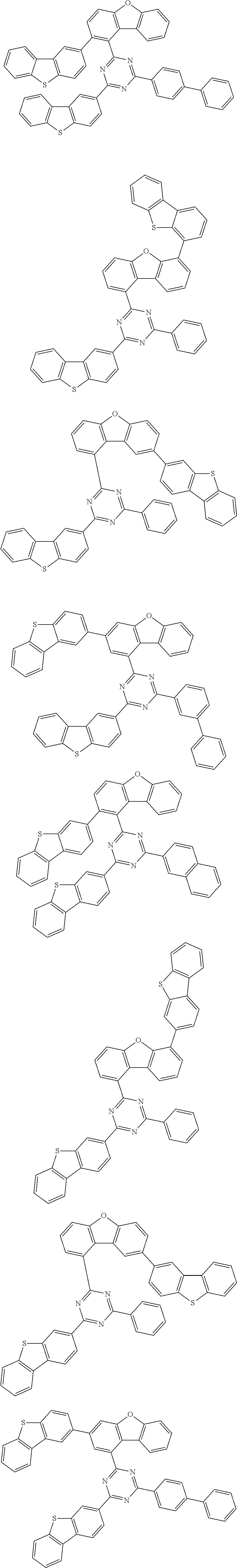

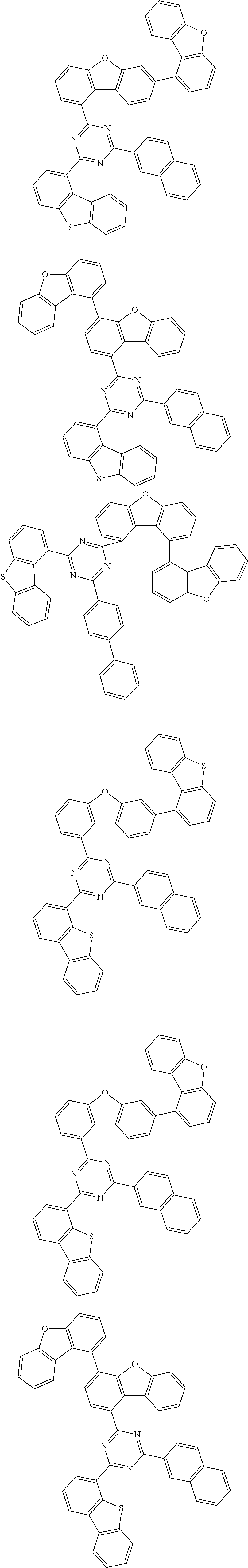

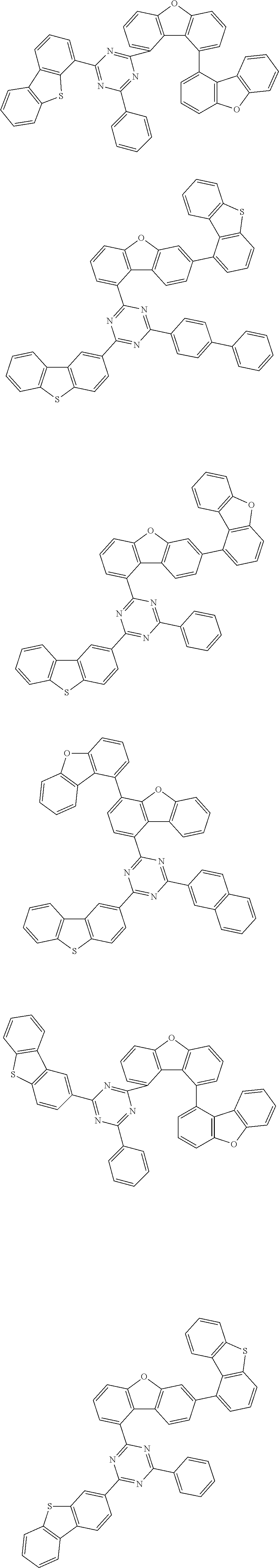

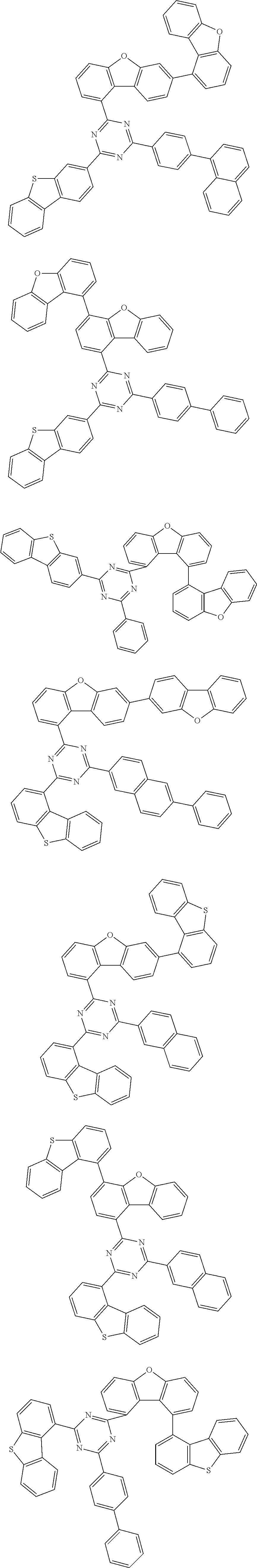

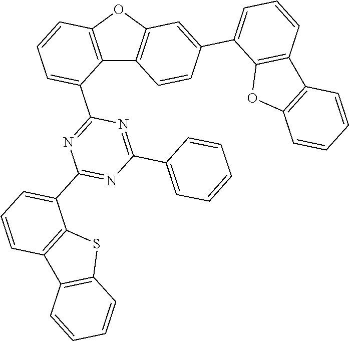 [SMILES: c1ccc(-c2nc(-c3cccc4c3sc3ccccc34)nc(-c3cccc4oc5cc(-c6cccc7c6oc6ccccc67)ccc5c34)n2)cc1]

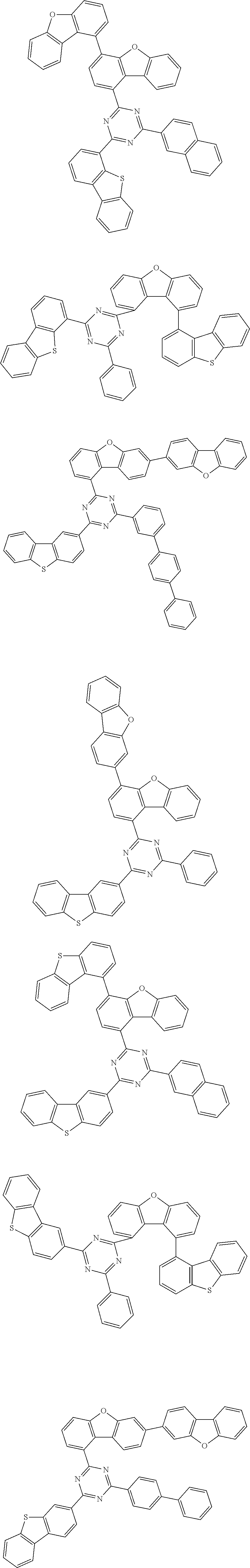

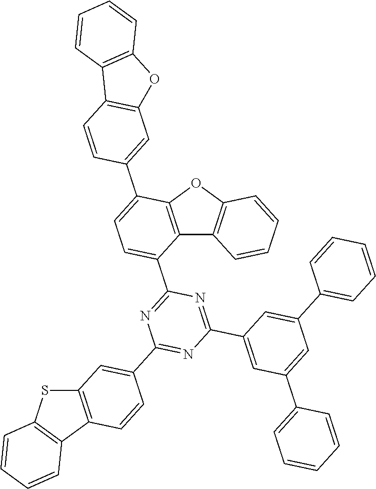 [SMILES: c1ccc(-c2cc(-c3ccccc3)cc(-c3nc(-c4ccc5c(c4)sc4ccccc45)nc(-c4ccc(-c5ccc6c(c5)oc5ccccc56)c5oc6ccccc6c45)n3)c2)cc1]

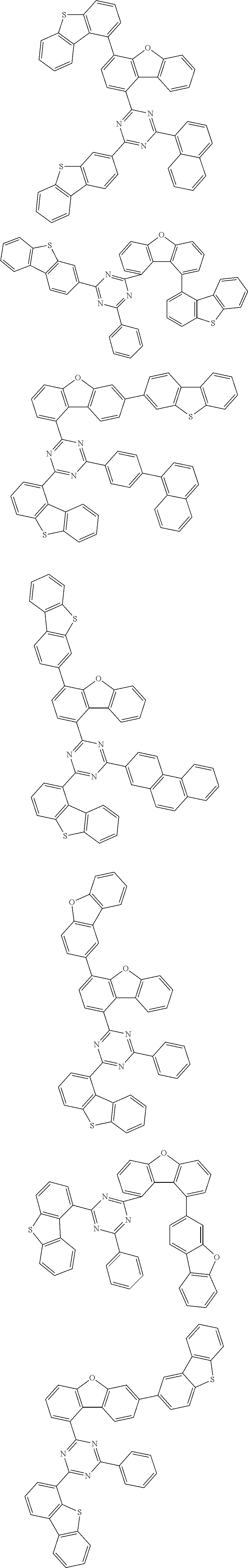

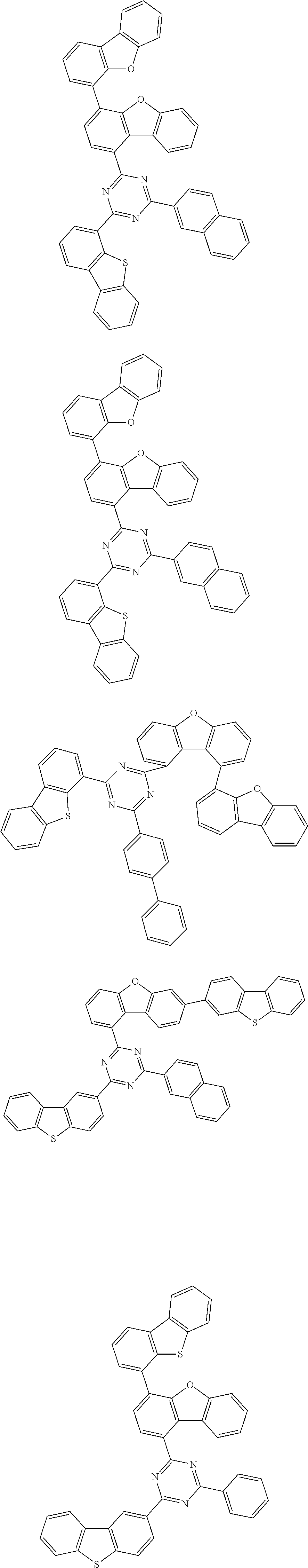

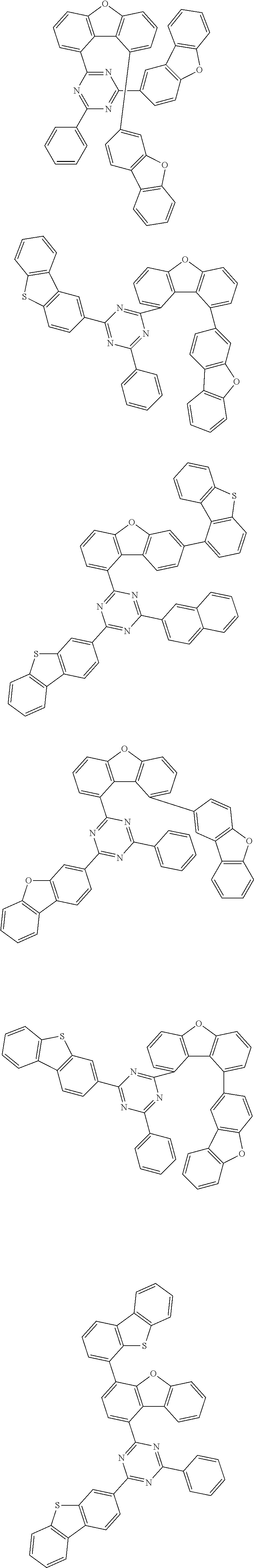

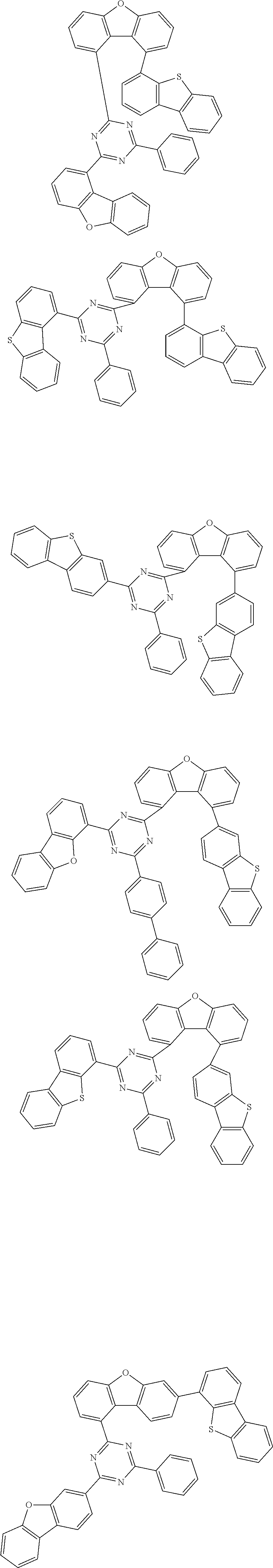

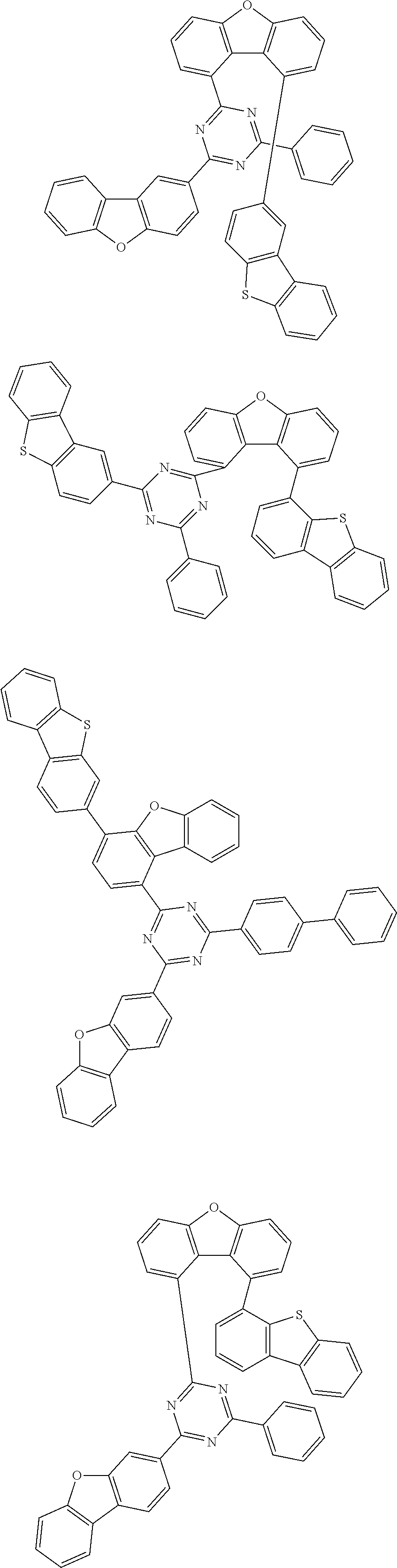 [SMILES: c1ccc(-c2nc(-c3ccc4oc5ccccc5c4c3)nc(-c3cccc4oc5cccc(-c6ccc7sc8ccccc8c7c6)c5c34)n2)cc1 ; c1ccc(-c2nc(-c3ccc4sc5ccccc5c4c3)nc(-c3cccc4oc5cccc(-c6cccc7c6sc6ccccc67)c5c34)n2)cc1 ; c1ccc(-c2ccc(-c3nc(-c4ccc5c(c4)oc4ccccc45)nc(-c4ccc(-c5ccc6c(c5)sc5ccccc56)c5oc6ccccc6c45)n3)cc2)cc1 ; c1ccc(-c2nc(-c3ccc4c(c3)oc3ccccc34)nc(-c3cccc4oc5cccc(-c6cccc7c6sc6ccccc67)c5c34)n2)cc1]

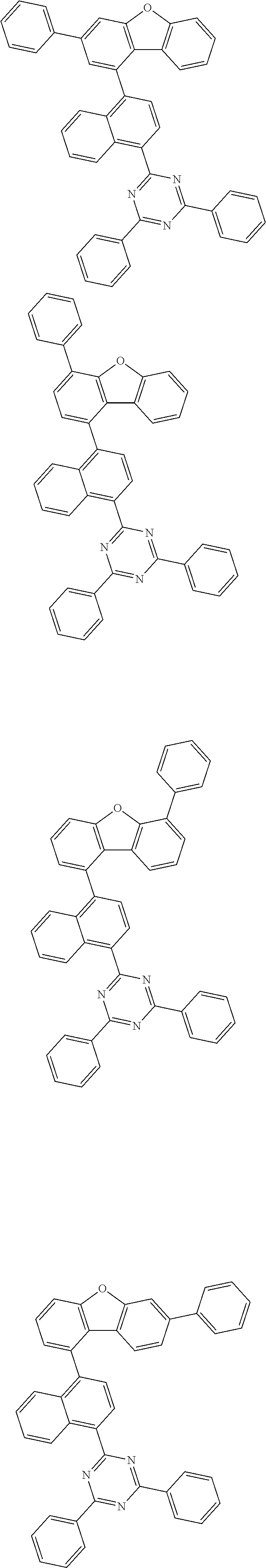

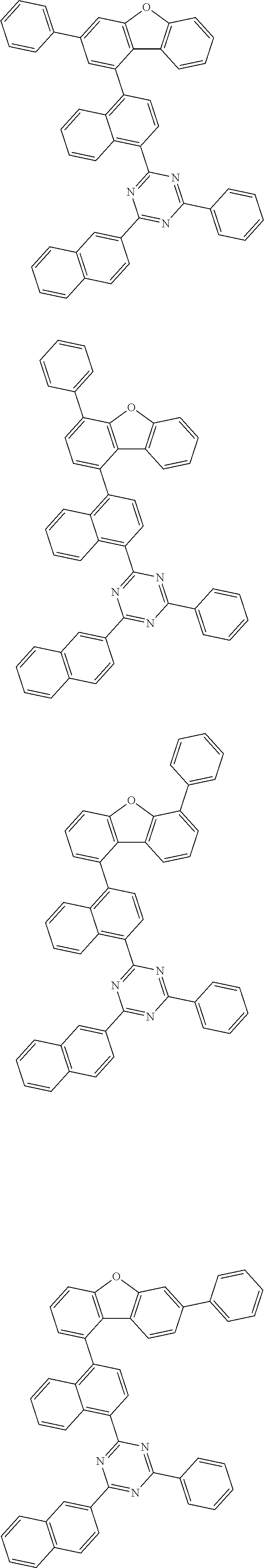 [SMILES: c1ccc(-c2cc(-c3ccc(-c4nc(-c5ccccc5)nc(-c5ccc6ccccc6c5)n4)c4ccccc34)c3c(c2)oc2ccccc23)cc1 ; c1ccc(-c2nc(-c3ccc4ccccc4c3)nc(-c3ccc(-c4ccc(-c5ccccc5)c5oc6ccccc6c45)c4ccccc34)n2)cc1 ; c1ccc(-c2nc(-c3ccc4ccccc4c3)nc(-c3ccc(-c4cccc5oc6c(-c7ccccc7)cccc6c45)c4ccccc34)n2)cc1 ; c1ccc(-c2ccc3c(c2)oc2cccc(-c4ccc(-c5nc(-c6ccccc6)nc(-c6ccc7ccccc7c6)n5)c5ccccc45)c23)cc1]

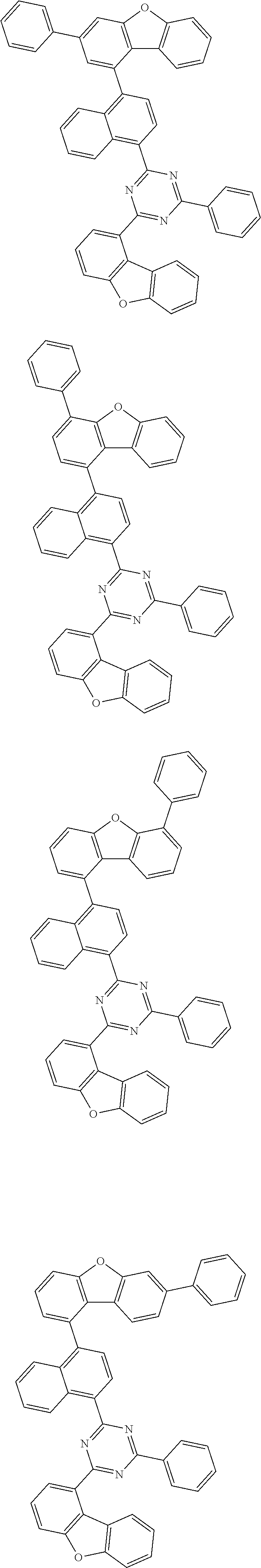 [SMILES: c1ccc(-c2cc(-c3ccc(-c4nc(-c5ccccc5)nc(-c5cccc6oc7ccccc7c56)n4)c4ccccc34)c3c(c2)oc2ccccc23)cc1 ; c1ccc(-c2nc(-c3ccc(-c4ccc(-c5ccccc5)c5oc6ccccc6c45)c4ccccc34)nc(-c3cccc4oc5ccccc5c34)n2)cc1 ; c1ccc(-c2nc(-c3ccc(-c4cccc5oc6c(-c7ccccc7)cccc6c45)c4ccccc34)nc(-c3cccc4oc5ccccc5c34)n2)cc1 ; c1ccc(-c2ccc3c(c2)oc2cccc(-c4ccc(-c5nc(-c6ccccc6)nc(-c6cccc7oc8ccccc8c67)n5)c5ccccc45)c23)cc1]

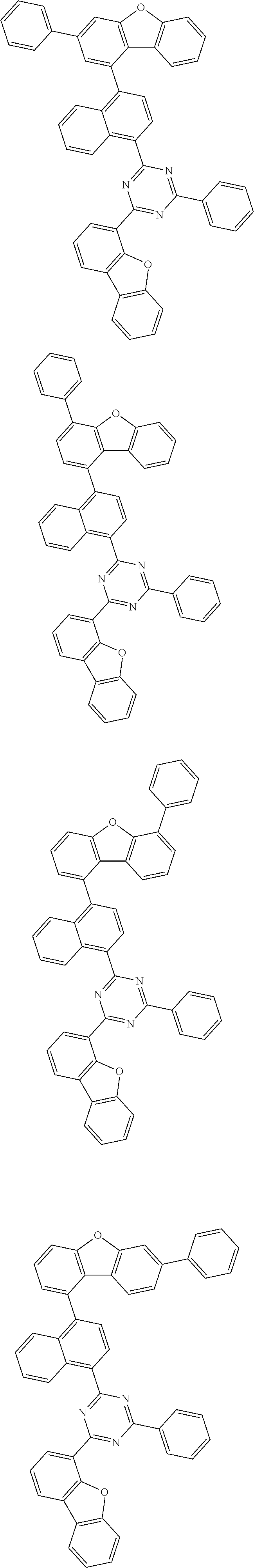 [SMILES: c1ccc(-c2cc(-c3ccc(-c4nc(-c5ccccc5)nc(-c5cccc6c5oc5ccccc56)n4)c4ccccc34)c3c(c2)oc2ccccc23)cc1 ; c1ccc(-c2nc(-c3ccc(-c4ccc(-c5ccccc5)c5oc6ccccc6c45)c4ccccc34)nc(-c3cccc4c3oc3ccccc34)n2)cc1 ; c1ccc(-c2nc(-c3ccc(-c4cccc5oc6c(-c7ccccc7)cccc6c45)c4ccccc34)nc(-c3cccc4c3oc3ccccc34)n2)cc1 ; c1ccc(-c2ccc3c(c2)oc2cccc(-c4ccc(-c5nc(-c6ccccc6)nc(-c6cccc7c6oc6ccccc67)n5)c5ccccc45)c23)cc1]

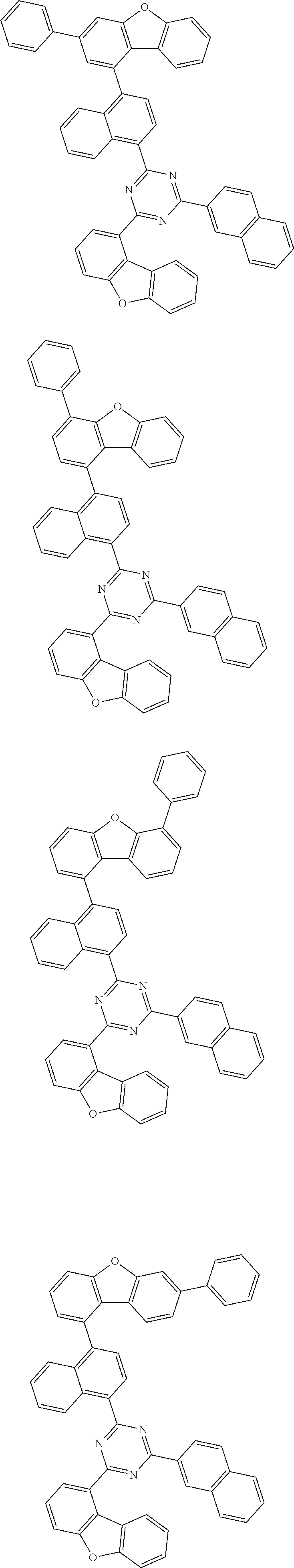 [SMILES: c1ccc(-c2cc(-c3ccc(-c4nc(-c5ccc6ccccc6c5)nc(-c5cccc6oc7ccccc7c56)n4)c4ccccc34)c3c(c2)oc2ccccc23)cc1 ; c1ccc(-c2ccc(-c3ccc(-c4nc(-c5ccc6ccccc6c5)nc(-c5cccc6oc7ccccc7c56)n4)c4ccccc34)c3c2oc2ccccc23)cc1 ; c1ccc(-c2cccc3c2oc2cccc(-c4ccc(-c5nc(-c6ccc7ccccc7c6)nc(-c6cccc7oc8ccccc8c67)n5)c5ccccc45)c23)cc1 ; c1ccc(-c2ccc3c(c2)oc2cccc(-c4ccc(-c5nc(-c6ccc7ccccc7c6)nc(-c6cccc7oc8ccccc8c67)n5)c5ccccc45)c23)cc1]

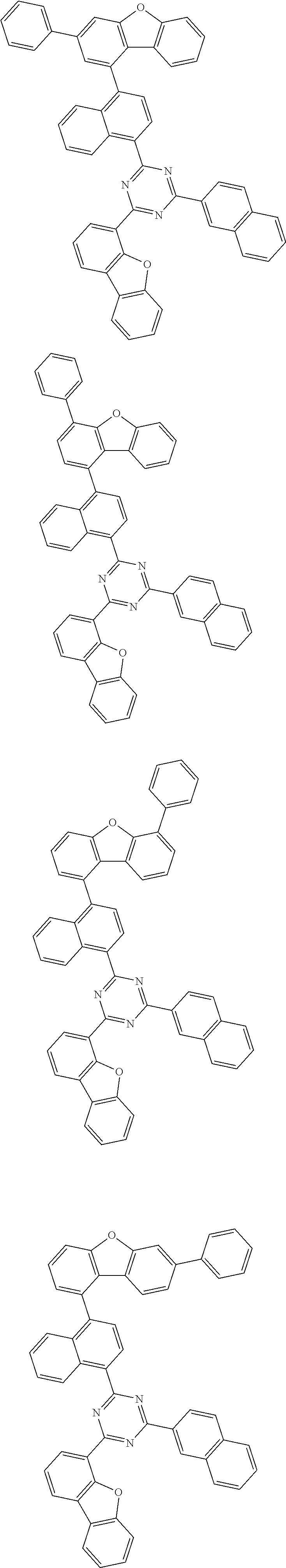 [SMILES: c1ccc(-c2cc(-c3ccc(-c4nc(-c5ccc6ccccc6c5)nc(-c5cccc6c5oc5ccccc56)n4)c4ccccc34)c3c(c2)oc2ccccc23)cc1 ; c1ccc(-c2ccc(-c3ccc(-c4nc(-c5ccc6ccccc6c5)nc(-c5cccc6c5oc5ccccc56)n4)c4ccccc34)c3c2oc2ccccc23)cc1 ; c1ccc(-c2cccc3c2oc2cccc(-c4ccc(-c5nc(-c6ccc7ccccc7c6)nc(-c6cccc7c6oc6ccccc67)n5)c5ccccc45)c23)cc1 ; c1ccc(-c2ccc3c(c2)oc2cccc(-c4ccc(-c5nc(-c6ccc7ccccc7c6)nc(-c6cccc7c6oc6ccccc67)n5)c5ccccc45)c23)cc1]

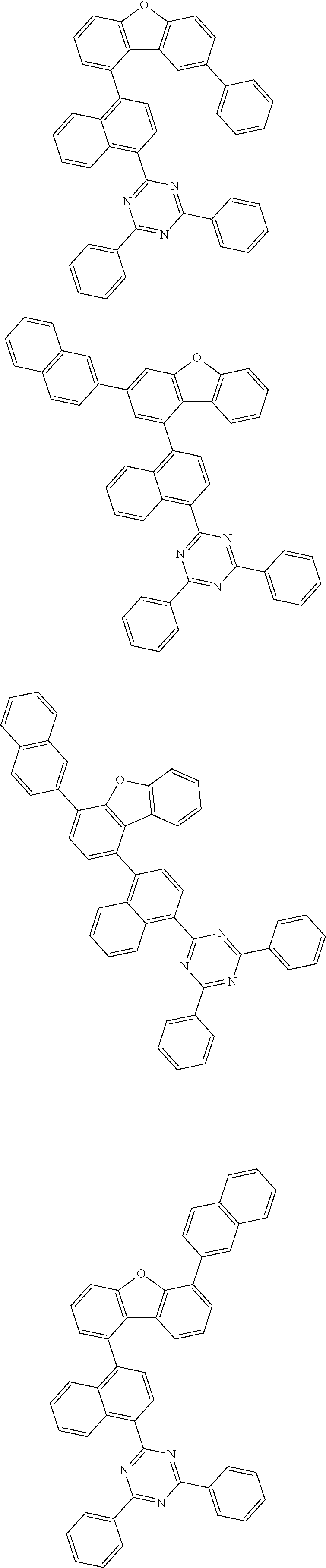

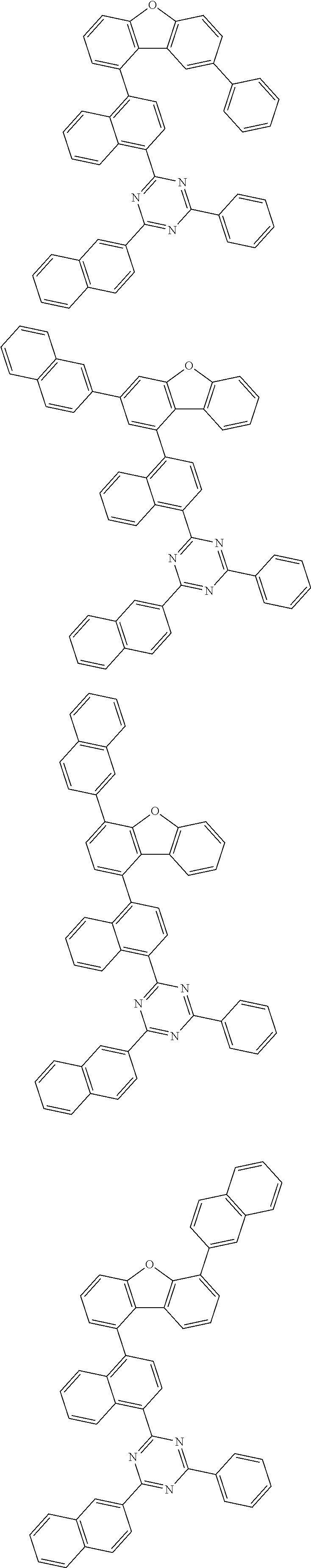

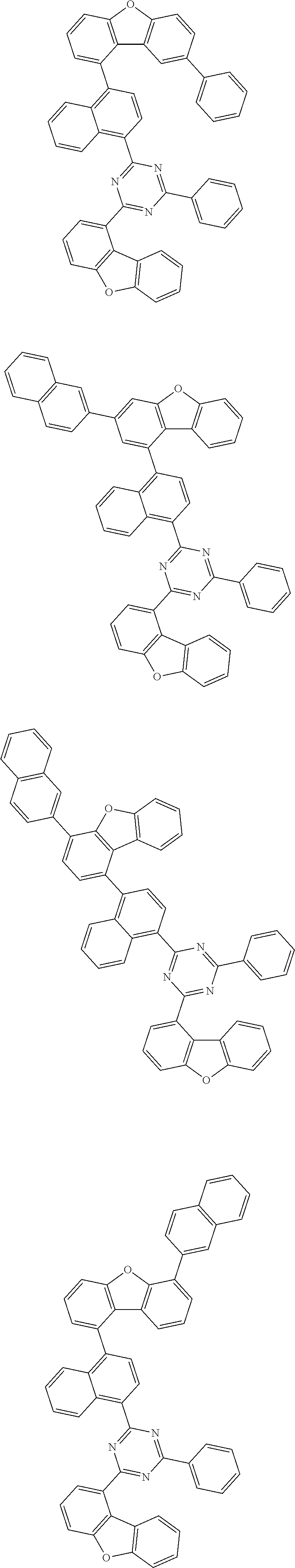 [SMILES: c1ccc(-c2ccc3oc4cccc(-c5ccc(-c6nc(-c7ccccc7)nc(-c7cccc8oc9ccccc9c78)n6)c6ccccc56)c4c3c2)cc1 ; c1ccc(-c2nc(-c3ccc(-c4cc(-c5ccc6ccccc6c5)cc5oc6ccccc6c45)c4ccccc34)nc(-c3cccc4oc5ccccc5c34)n2)cc1 ; c1ccc(-c2nc(-c3ccc(-c4ccc(-c5ccc6ccccc6c5)c5oc6ccccc6c45)c4ccccc34)nc(-c3cccc4oc5ccccc5c34)n2)cc1 ; c1ccc(-c2nc(-c3ccc(-c4cccc5oc6c(-c7ccc8ccccc8c7)cccc6c45)c4ccccc34)nc(-c3cccc4oc5ccccc5c34)n2)cc1]

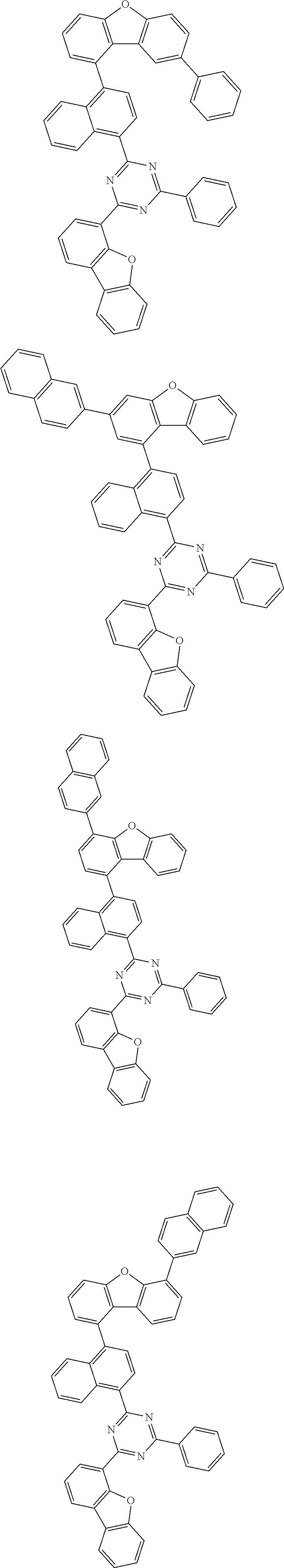 [SMILES: c1ccc(-c2ccc3oc4cccc(-c5ccc(-c6nc(-c7ccccc7)nc(-c7cccc8c7oc7ccccc78)n6)c6ccccc56)c4c3c2)cc1 ; c1ccc(-c2nc(-c3ccc(-c4cc(-c5ccc6ccccc6c5)cc5oc6ccccc6c45)c4ccccc34)nc(-c3cccc4c3oc3ccccc34)n2)cc1 ; c1ccc(-c2nc(-c3ccc(-c4ccc(-c5ccc6ccccc6c5)c5oc6ccccc6c45)c4ccccc34)nc(-c3cccc4c3oc3ccccc34)n2)cc1 ; c1ccc(-c2nc(-c3ccc(-c4cccc5oc6c(-c7ccc8ccccc8c7)cccc6c45)c4ccccc34)nc(-c3cccc4c3oc3ccccc34)n2)cc1]

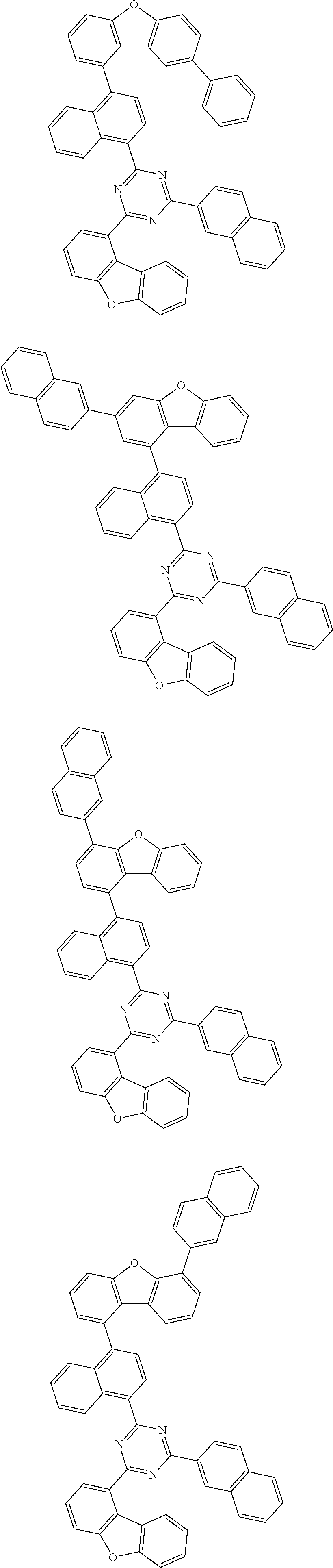 [SMILES: c1ccc(-c2ccc3oc4cccc(-c5ccc(-c6nc(-c7ccc8ccccc8c7)nc(-c7cccc8oc9ccccc9c78)n6)c6ccccc56)c4c3c2)cc1 ; c1ccc2cc(-c3cc(-c4ccc(-c5nc(-c6ccc7ccccc7c6)nc(-c6cccc7oc8ccccc8c67)n5)c5ccccc45)c4c(c3)oc3ccccc34)ccc2c1 ; c1ccc2cc(-c3nc(-c4ccc(-c5ccc(-c6ccc7ccccc7c6)c6oc7ccccc7c56)c5ccccc45)nc(-c4cccc5oc6ccccc6c45)n3)ccc2c1 ; c1ccc2cc(-c3nc(-c4ccc(-c5cccc6oc7c(-c8ccc9ccccc9c8)cccc7c56)c5ccccc45)nc(-c4cccc5oc6ccccc6c45)n3)ccc2c1]

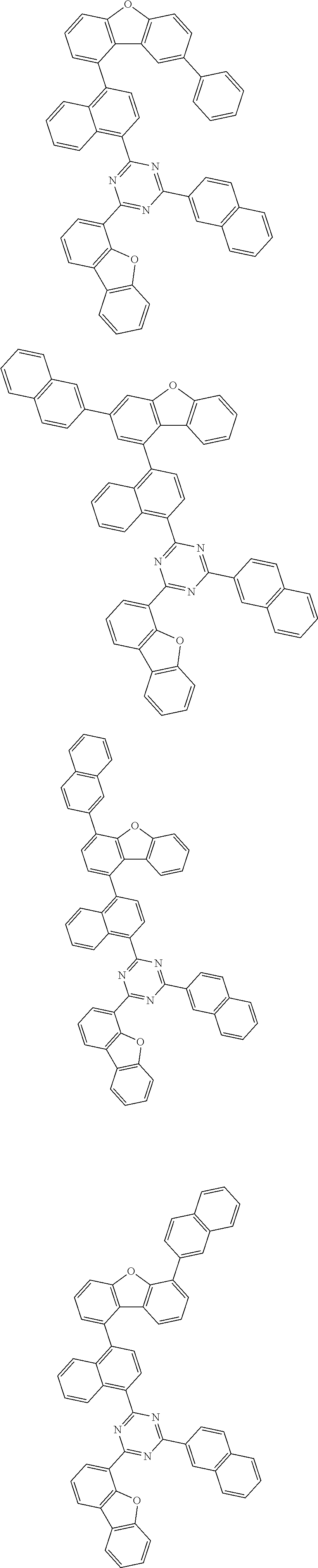 [SMILES: c1ccc(-c2ccc3oc4cccc(-c5ccc(-c6nc(-c7ccc8ccccc8c7)nc(-c7cccc8c7oc7ccccc78)n6)c6ccccc56)c4c3c2)cc1 ; c1ccc2cc(-c3cc(-c4ccc(-c5nc(-c6ccc7ccccc7c6)nc(-c6cccc7c6oc6ccccc67)n5)c5ccccc45)c4c(c3)oc3ccccc34)ccc2c1 ; c1ccc2cc(-c3nc(-c4ccc(-c5ccc(-c6ccc7ccccc7c6)c6oc7ccccc7c56)c5ccccc45)nc(-c4cccc5c4oc4ccccc45)n3)ccc2c1 ; c1ccc2cc(-c3nc(-c4ccc(-c5cccc6oc7c(-c8ccc9ccccc9c8)cccc7c56)c5ccccc45)nc(-c4cccc5c4oc4ccccc45)n3)ccc2c1]

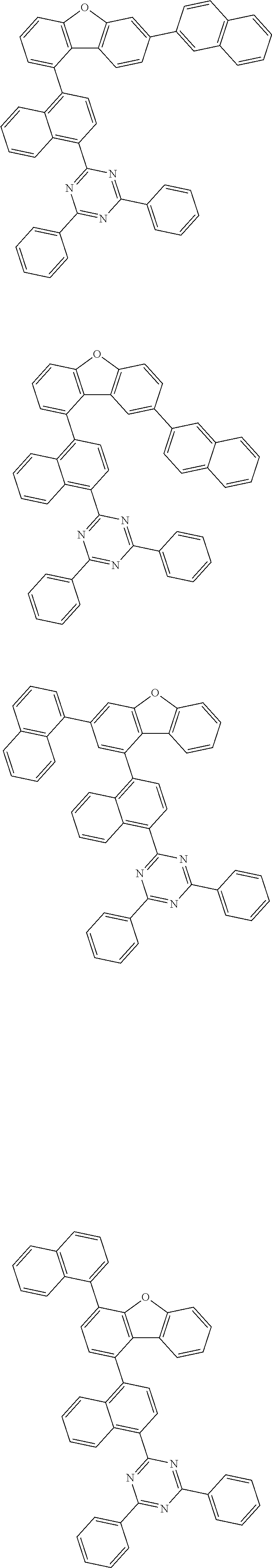 [SMILES: c1ccc(-c2nc(-c3ccccc3)nc(-c3ccc(-c4cccc5oc6cc(-c7ccc8ccccc8c7)ccc6c45)c4ccccc34)n2)cc1 ; c1ccc(-c2nc(-c3ccccc3)nc(-c3ccc(-c4cccc5oc6ccc(-c7ccc8ccccc8c7)cc6c45)c4ccccc34)n2)cc1 ; c1ccc(-c2nc(-c3ccccc3)nc(-c3ccc(-c4cc(-c5cccc6ccccc56)cc5oc6ccccc6c45)c4ccccc34)n2)cc1 ; c1ccc(-c2nc(-c3ccccc3)nc(-c3ccc(-c4ccc(-c5cccc6ccccc56)c5oc6ccccc6c45)c4ccccc34)n2)cc1]

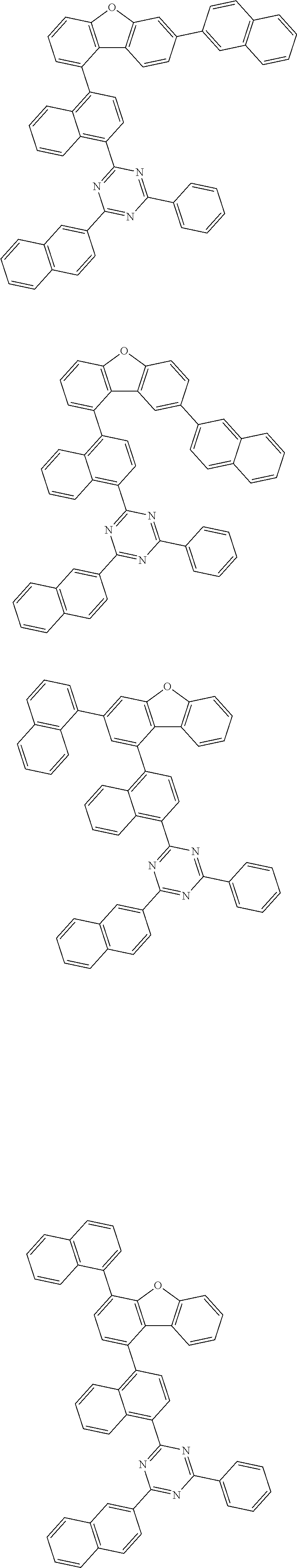 [SMILES: c1ccc(-c2nc(-c3ccc4ccccc4c3)nc(-c3ccc(-c4cccc5oc6cc(-c7ccc8ccccc8c7)ccc6c45)c4ccccc34)n2)cc1 ; c1ccc(-c2nc(-c3ccc4ccccc4c3)nc(-c3ccc(-c4cccc5oc6ccc(-c7ccc8ccccc8c7)cc6c45)c4ccccc34)n2)cc1 ; c1ccc(-c2nc(-c3ccc4ccccc4c3)nc(-c3ccc(-c4cc(-c5cccc6ccccc56)cc5oc6ccccc6c45)c4ccccc34)n2)cc1 ; c1ccc(-c2nc(-c3ccc4ccccc4c3)nc(-c3ccc(-c4ccc(-c5cccc6ccccc56)c5oc6ccccc6c45)c4ccccc34)n2)cc1]

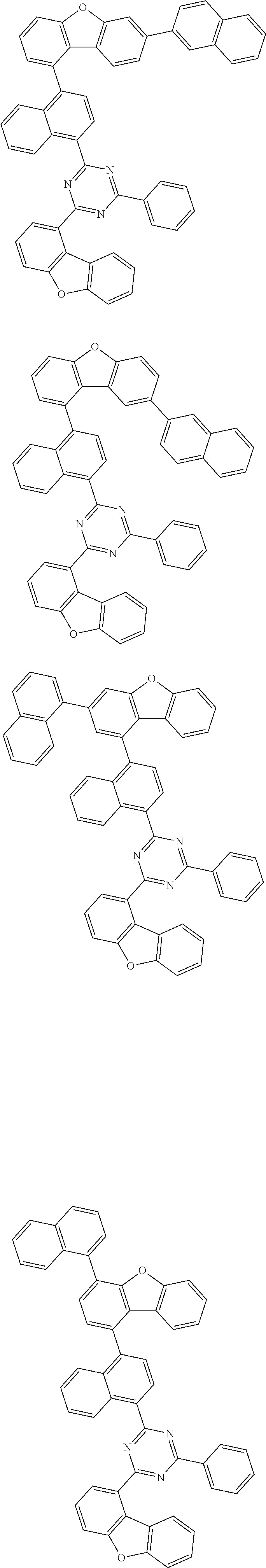 [SMILES: c1ccc(-c2nc(-c3ccc(-c4cccc5oc6cc(-c7ccc8ccccc8c7)ccc6c45)c4ccccc34)nc(-c3cccc4oc5ccccc5c34)n2)cc1 ; c1ccc(-c2nc(-c3ccc(-c4cccc5oc6ccc(-c7ccc8ccccc8c7)cc6c45)c4ccccc34)nc(-c3cccc4oc5ccccc5c34)n2)cc1 ; c1ccc(-c2nc(-c3ccc(-c4cc(-c5cccc6ccccc56)cc5oc6ccccc6c45)c4ccccc34)nc(-c3cccc4oc5ccccc5c34)n2)cc1 ; c1ccc(-c2nc(-c3ccc(-c4ccc(-c5cccc6ccccc56)c5oc6ccccc6c45)c4ccccc34)nc(-c3cccc4oc5ccccc5c34)n2)cc1]

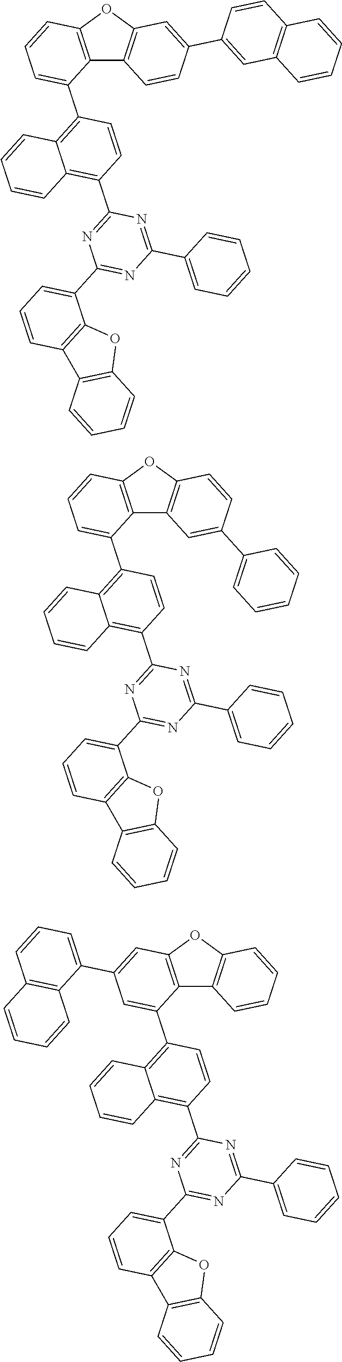

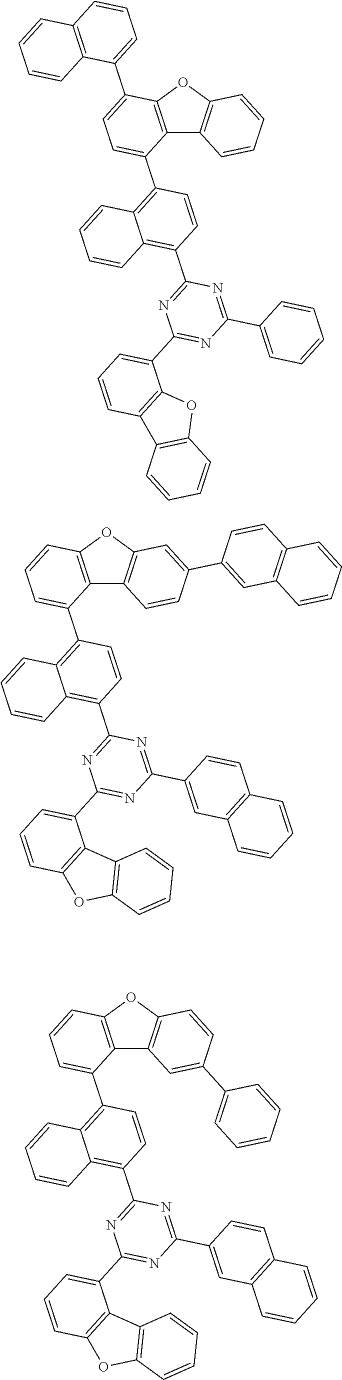

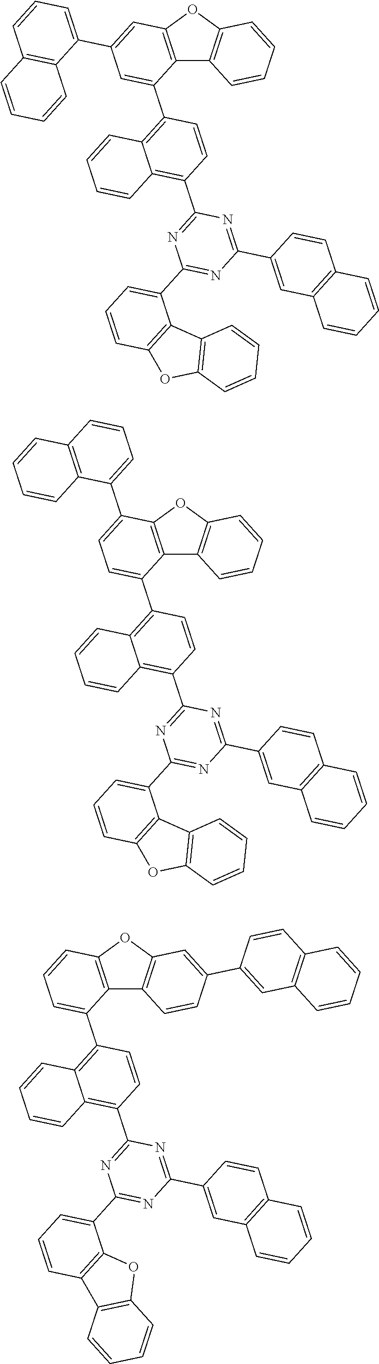 [SMILES: c1ccc2cc(-c3nc(-c4ccc(-c5cc(-c6cccc7ccccc67)cc6oc7ccccc7c56)c5ccccc45)nc(-c4cccc5oc6ccccc6c45)n3)ccc2c1 ; c1ccc2cc(-c3nc(-c4ccc(-c5ccc(-c6cccc7ccccc67)c6oc7ccccc7c56)c5ccccc45)nc(-c4cccc5oc6ccccc6c45)n3)ccc2c1 ; c1ccc2cc(-c3ccc4c(c3)oc3cccc(-c5ccc(-c6nc(-c7ccc8ccccc8c7)nc(-c7cccc8c7oc7ccccc78)n6)c6ccccc56)c34)ccc2c1]

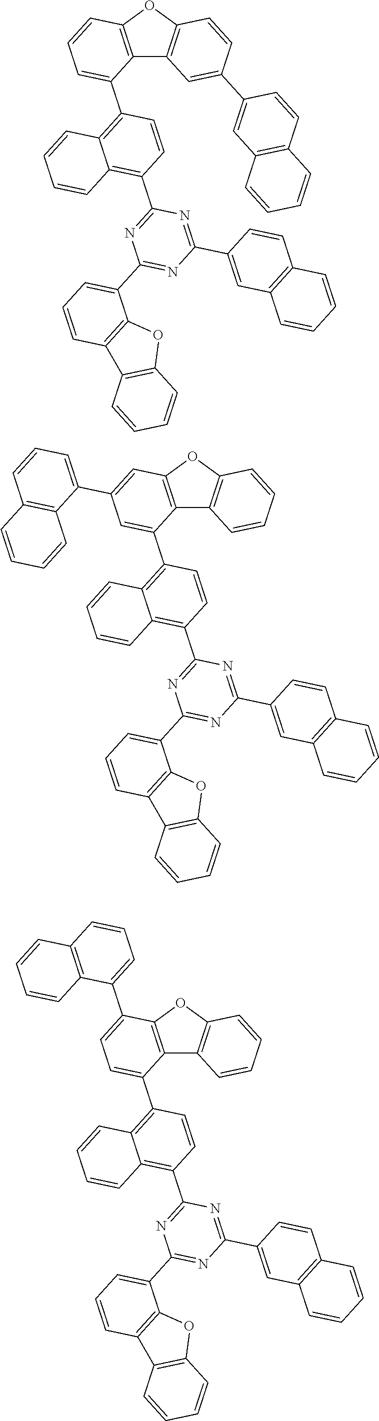

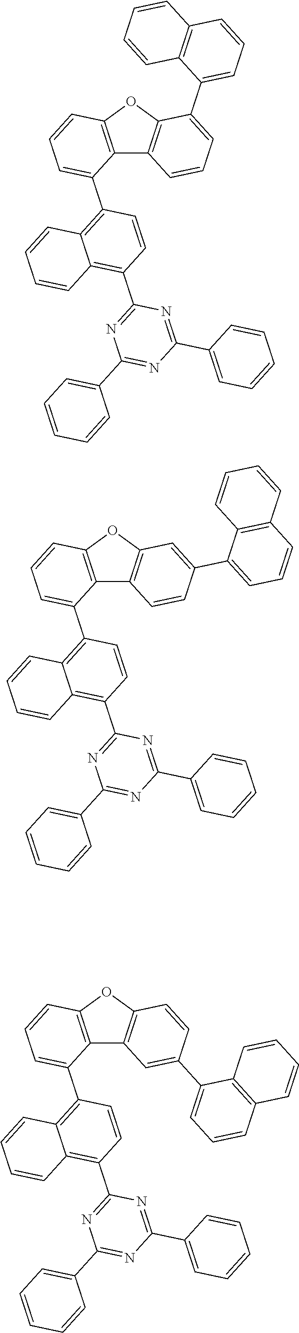 [SMILES: c1ccc(-c2nc(-c3ccccc3)nc(-c3ccc(-c4cccc5oc6c(-c7cccc8ccccc78)cccc6c45)c4ccccc34)n2)cc1 ; c1ccc(-c2nc(-c3ccccc3)nc(-c3ccc(-c4cccc5oc6cc(-c7cccc8ccccc78)ccc6c45)c4ccccc34)n2)cc1 ; c1ccc(-c2nc(-c3ccccc3)nc(-c3ccc(-c4cccc5oc6ccc(-c7cccc8ccccc78)cc6c45)c4ccccc34)n2)cc1]

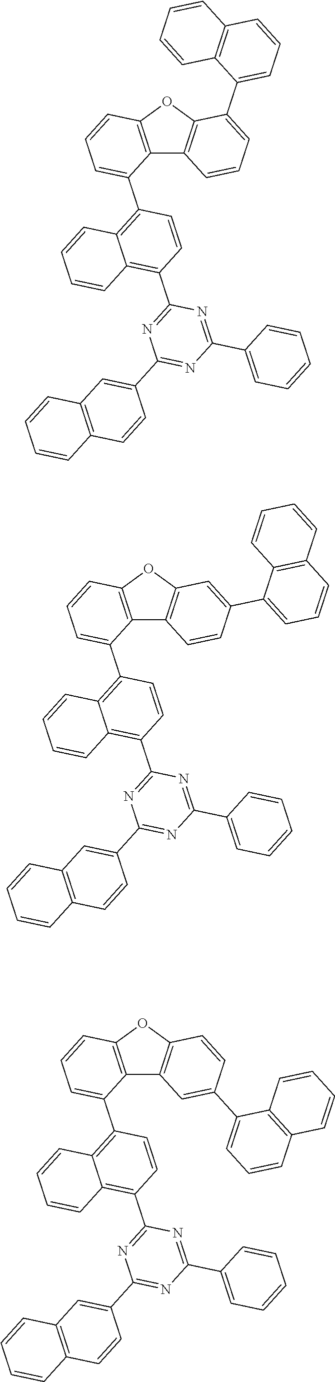 [SMILES: c1ccc(-c2nc(-c3ccc4ccccc4c3)nc(-c3ccc(-c4cccc5oc6c(-c7cccc8ccccc78)cccc6c45)c4ccccc34)n2)cc1 ; c1ccc(-c2nc(-c3ccc4ccccc4c3)nc(-c3ccc(-c4cccc5oc6cc(-c7cccc8ccccc78)ccc6c45)c4ccccc34)n2)cc1 ; c1ccc(-c2nc(-c3ccc4ccccc4c3)nc(-c3ccc(-c4cccc5oc6ccc(-c7cccc8ccccc78)cc6c45)c4ccccc34)n2)cc1]

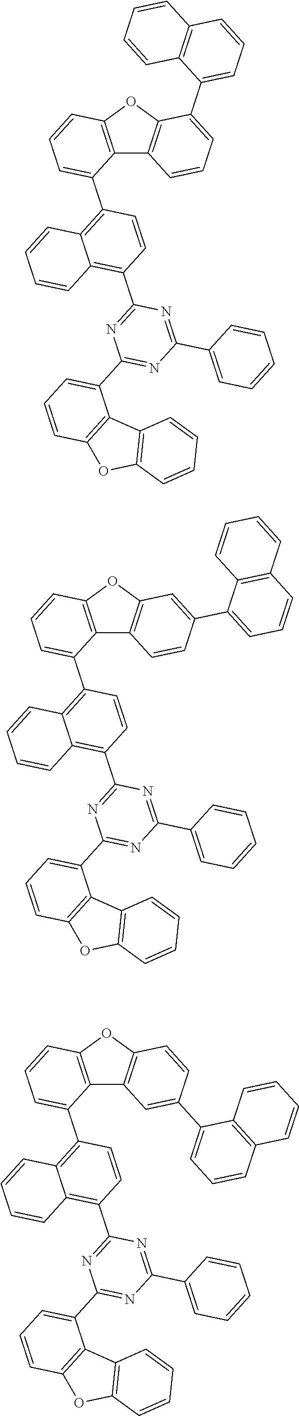 [SMILES: c1ccc(-c2nc(-c3ccc(-c4cccc5oc6c(-c7cccc8ccccc78)cccc6c45)c4ccccc34)nc(-c3cccc4oc5ccccc5c34)n2)cc1 ; c1ccc(-c2nc(-c3ccc(-c4cccc5oc6cc(-c7cccc8ccccc78)ccc6c45)c4ccccc34)nc(-c3cccc4oc5ccccc5c34)n2)cc1 ; c1ccc(-c2nc(-c3ccc(-c4cccc5oc6ccc(-c7cccc8ccccc78)cc6c45)c4ccccc34)nc(-c3cccc4oc5ccccc5c34)n2)cc1]

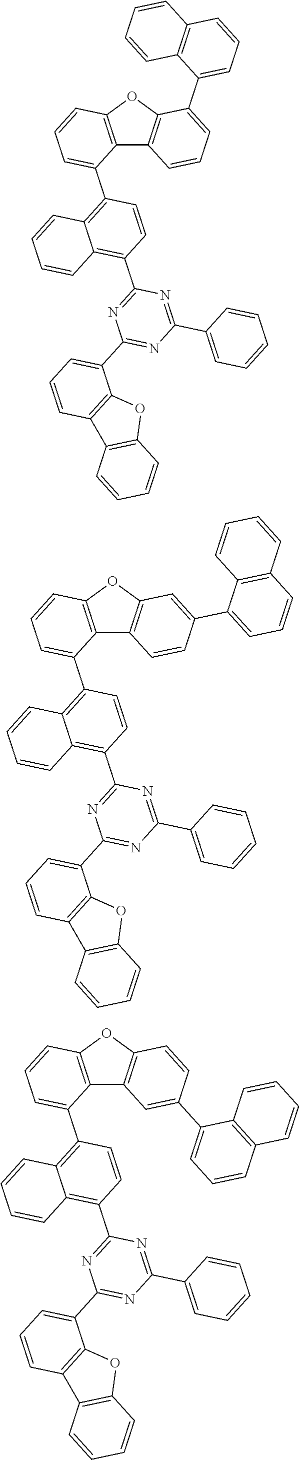

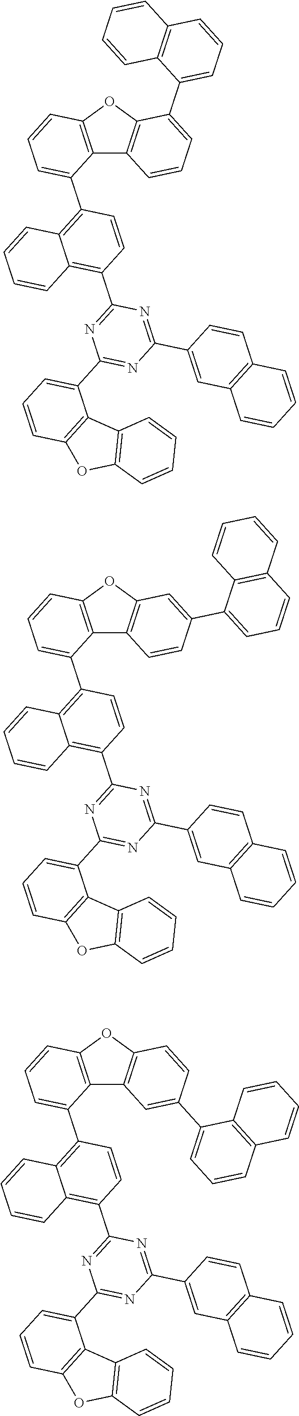 [SMILES: c1ccc2cc(-c3nc(-c4ccc(-c5cccc6oc7c(-c8cccc9ccccc89)cccc7c56)c5ccccc45)nc(-c4cccc5oc6ccccc6c45)n3)ccc2c1 ; c1ccc2cc(-c3nc(-c4ccc(-c5cccc6oc7cc(-c8cccc9ccccc89)ccc7c56)c5ccccc45)nc(-c4cccc5oc6ccccc6c45)n3)ccc2c1 ; c1ccc2cc(-c3nc(-c4ccc(-c5cccc6oc7ccc(-c8cccc9ccccc89)cc7c56)c5ccccc45)nc(-c4cccc5oc6ccccc6c45)n3)ccc2c1]

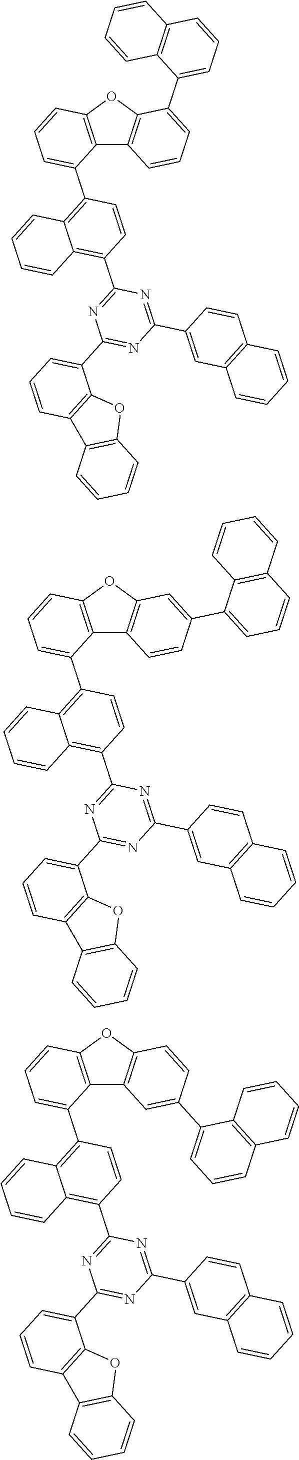

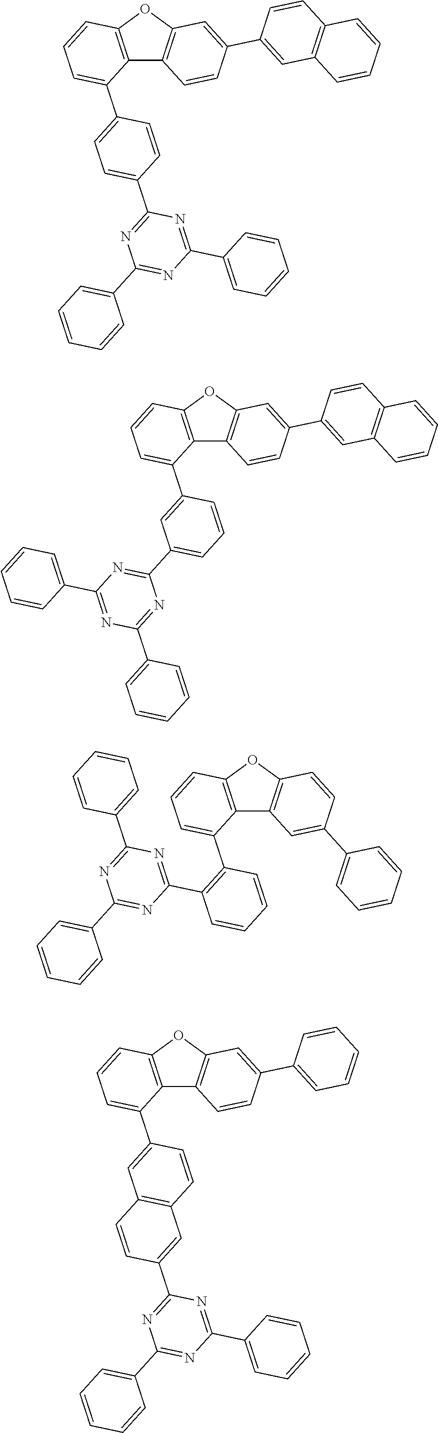

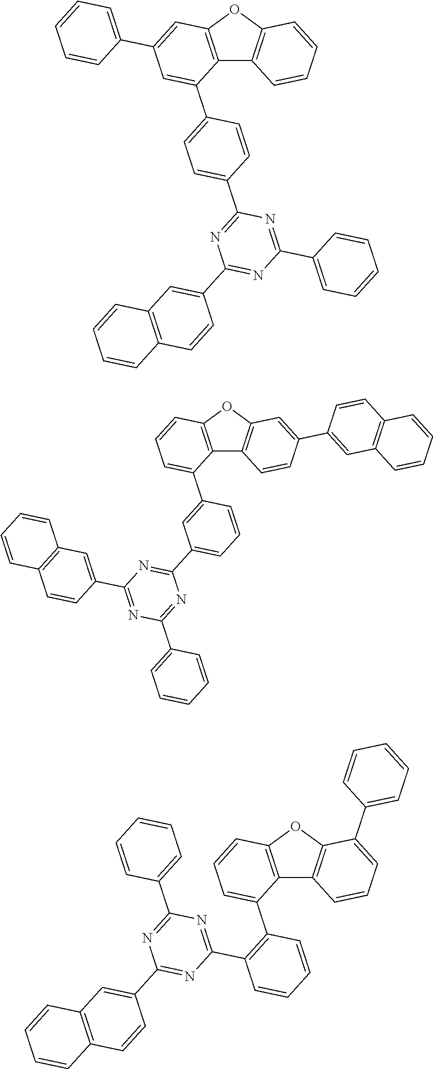 [SMILES: c1ccc(-c2cc(-c3ccc(-c4nc(-c5ccccc5)nc(-c5ccc6ccccc6c5)n4)cc3)c3c(c2)oc2ccccc23)cc1 ; c1ccc(-c2nc(-c3cccc(-c4cccc5oc6cc(-c7ccc8ccccc8c7)ccc6c45)c3)nc(-c3ccc4ccccc4c3)n2)cc1 ; c1ccc(-c2nc(-c3ccc4ccccc4c3)nc(-c3ccccc3-c3cccc4oc5c(-c6ccccc6)cccc5c34)n2)cc1]

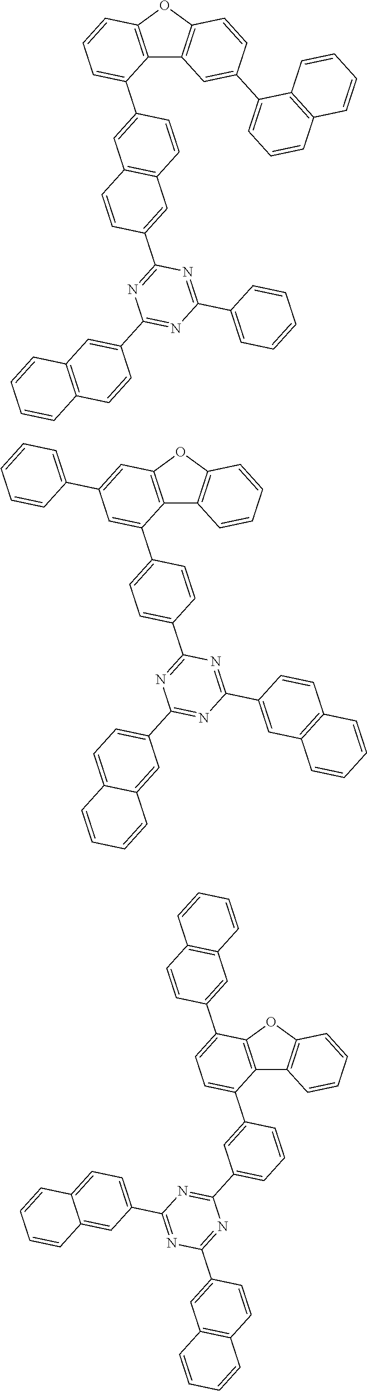

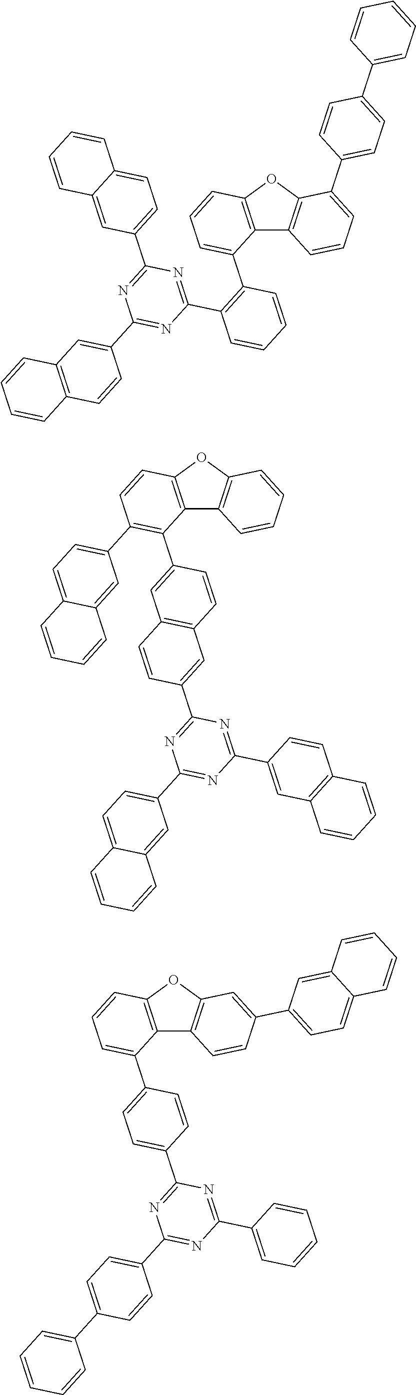 [SMILES: c1ccc(-c2ccc(-c3cccc4c3oc3cccc(-c5ccccc5-c5nc(-c6ccc7ccccc7c6)nc(-c6ccc7ccccc7c6)n5)c34)cc2)cc1 ; c1ccc2cc(-c3nc(-c4ccc5ccccc5c4)nc(-c4ccc5cc(-c6c(-c7ccc8ccccc8c7)ccc7oc8ccccc8c67)ccc5c4)n3)ccc2c1 ; c1ccc(-c2ccc(-c3nc(-c4ccccc4)nc(-c4ccc(-c5cccc6oc7cc(-c8ccc9ccccc9c8)ccc7c56)cc4)n3)cc2)cc1]

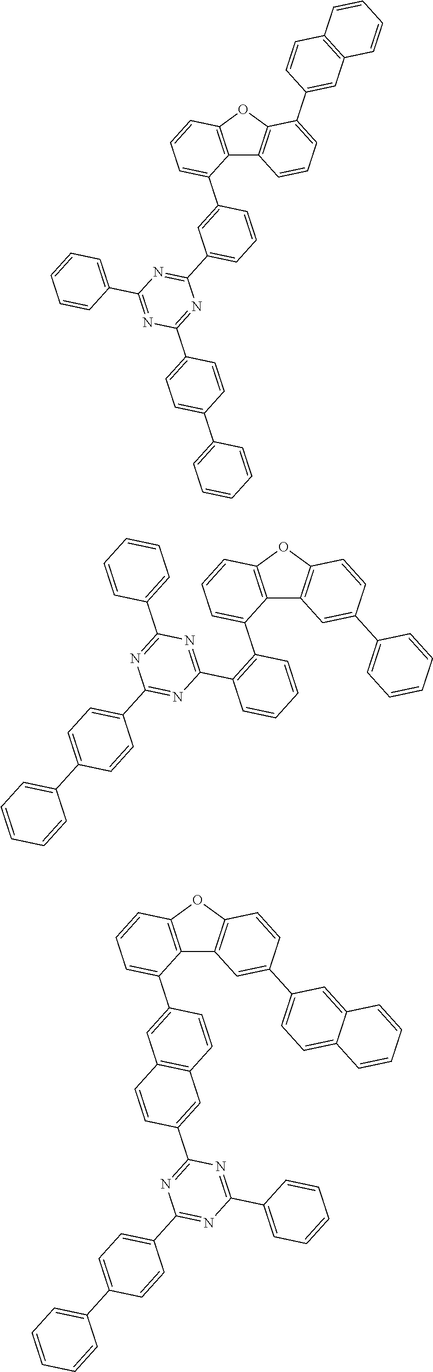 [SMILES: c1ccc(-c2ccc(-c3nc(-c4ccccc4)nc(-c4cccc(-c5cccc6oc7c(-c8ccc9ccccc9c8)cccc7c56)c4)n3)cc2)cc1 ; c1ccc(-c2ccc(-c3nc(-c4ccccc4)nc(-c4ccccc4-c4cccc5oc6ccc(-c7ccccc7)cc6c45)n3)cc2)cc1 ; c1ccc(-c2ccc(-c3nc(-c4ccccc4)nc(-c4ccc5cc(-c6cccc7oc8ccc(-c9ccc%10ccccc%10c9)cc8c67)ccc5c4)n3)cc2)cc1]

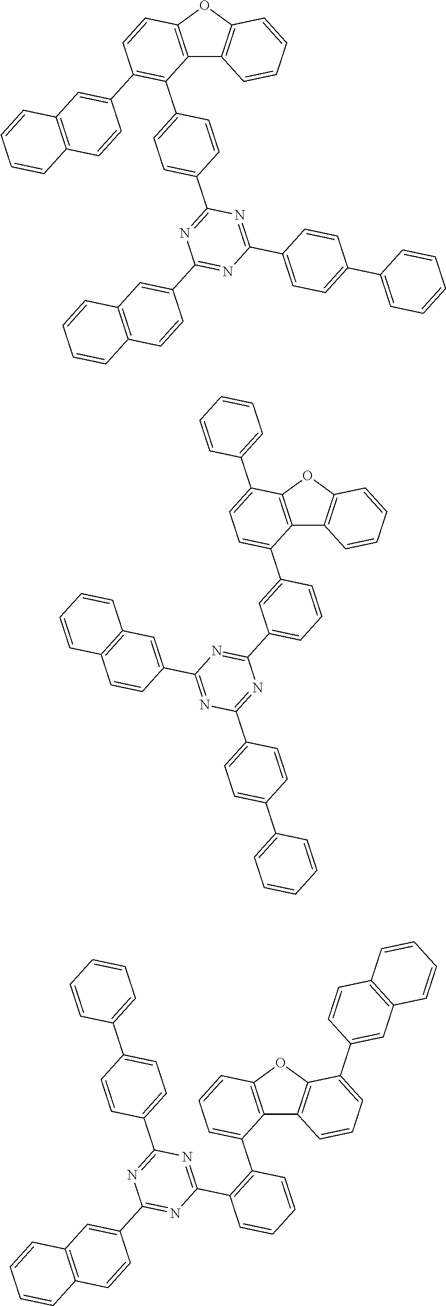 [SMILES: c1ccc(-c2ccc(-c3nc(-c4ccc(-c5c(-c6ccc7ccccc7c6)ccc6oc7ccccc7c56)cc4)nc(-c4ccc5ccccc5c4)n3)cc2)cc1 ; c1ccc(-c2ccc(-c3nc(-c4cccc(-c5ccc(-c6ccccc6)c6oc7ccccc7c56)c4)nc(-c4ccc5ccccc5c4)n3)cc2)cc1 ; c1ccc(-c2ccc(-c3nc(-c4ccc5ccccc5c4)nc(-c4ccccc4-c4cccc5oc6c(-c7ccc8ccccc8c7)cccc6c45)n3)cc2)cc1]

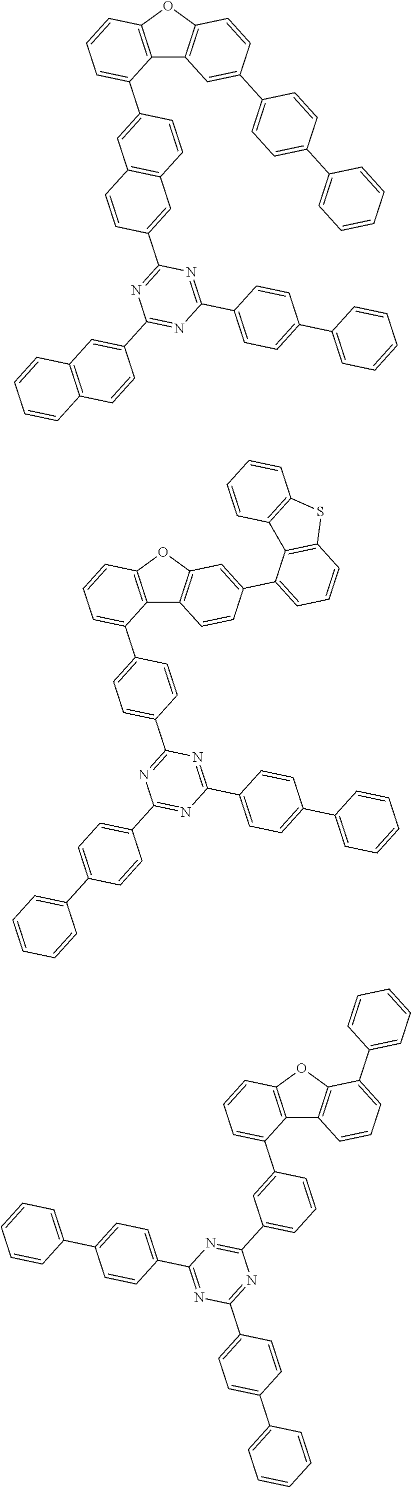 [SMILES: c1ccc(-c2ccc(-c3ccc4oc5cccc(-c6ccc7cc(-c8nc(-c9ccc(-c%10ccccc%10)cc9)nc(-c9ccc%10ccccc%10c9)n8)ccc7c6)c5c4c3)cc2)cc1 ; c1ccc(-c2ccc(-c3nc(-c4ccc(-c5ccccc5)cc4)nc(-c4ccc(-c5cccc6oc7cc(-c8cccc9sc%10ccccc%10c89)ccc7c56)cc4)n3)cc2)cc1 ; c1ccc(-c2ccc(-c3nc(-c4ccc(-c5ccccc5)cc4)nc(-c4cccc(-c5cccc6oc7c(-c8ccccc8)cccc7c56)c4)n3)cc2)cc1]

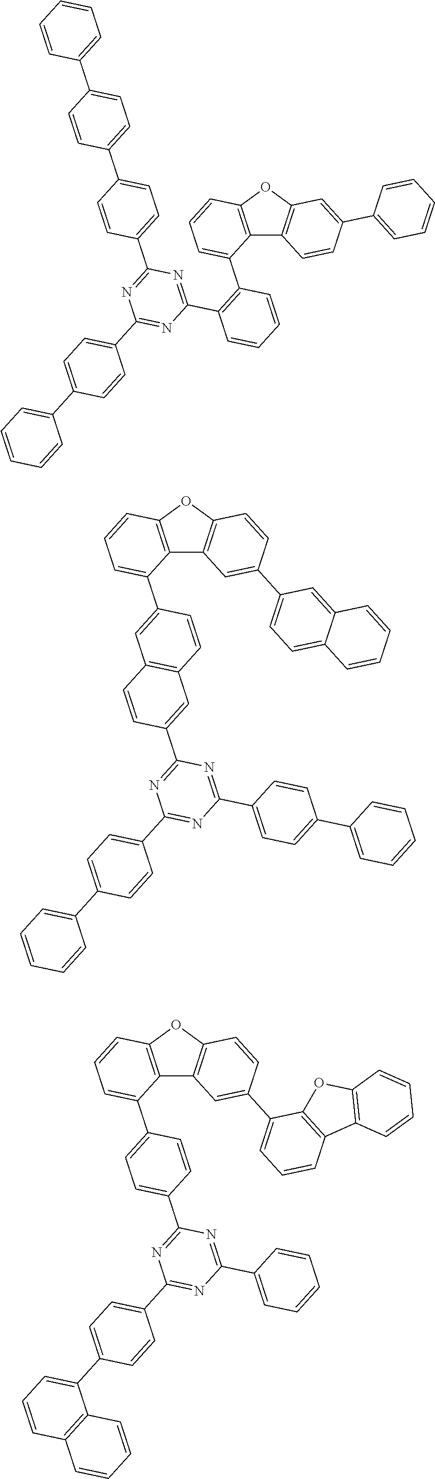 [SMILES: c1ccc(-c2ccc(-c3ccc(-c4nc(-c5ccc(-c6ccccc6)cc5)nc(-c5ccccc5-c5cccc6oc7cc(-c8ccccc8)ccc7c56)n4)cc3)cc2)cc1 ; c1ccc(-c2ccc(-c3nc(-c4ccc(-c5ccccc5)cc4)nc(-c4ccc5cc(-c6cccc7oc8ccc(-c9ccc%10ccccc%10c9)cc8c67)ccc5c4)n3)cc2)cc1 ; c1ccc(-c2nc(-c3ccc(-c4cccc5ccccc45)cc3)nc(-c3ccc(-c4cccc5oc6ccc(-c7cccc8c7oc7ccccc78)cc6c45)cc3)n2)cc1]

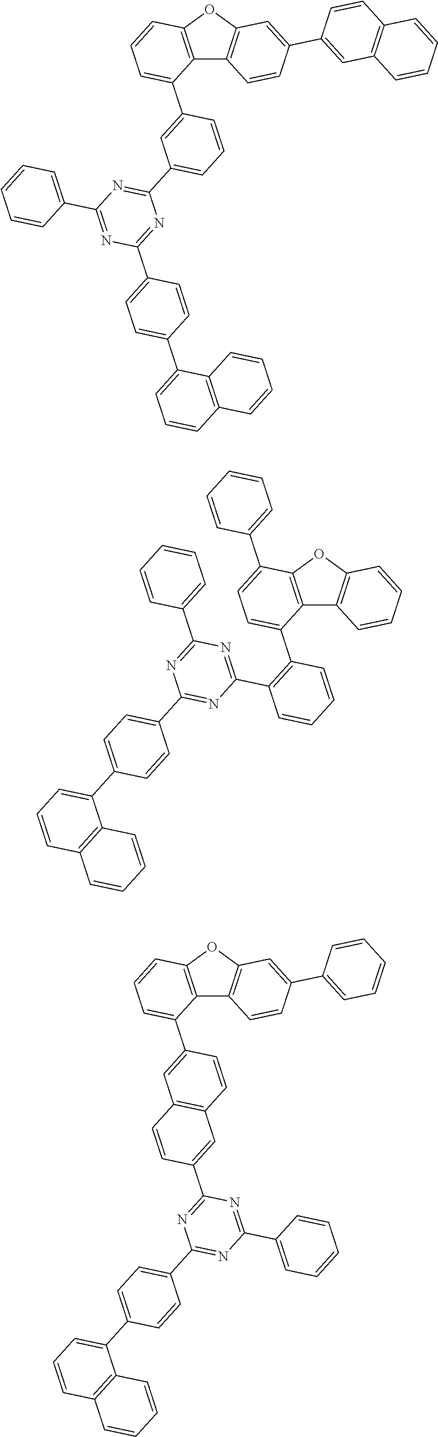 [SMILES: c1ccc(-c2nc(-c3ccc(-c4cccc5ccccc45)cc3)nc(-c3cccc(-c4cccc5oc6cc(-c7ccc8ccccc8c7)ccc6c45)c3)n2)cc1 ; c1ccc(-c2nc(-c3ccc(-c4cccc5ccccc45)cc3)nc(-c3ccccc3-c3ccc(-c4ccccc4)c4oc5ccccc5c34)n2)cc1 ; c1ccc(-c2ccc3c(c2)oc2cccc(-c4ccc5cc(-c6nc(-c7ccccc7)nc(-c7ccc(-c8cccc9ccccc89)cc7)n6)ccc5c4)c23)cc1]

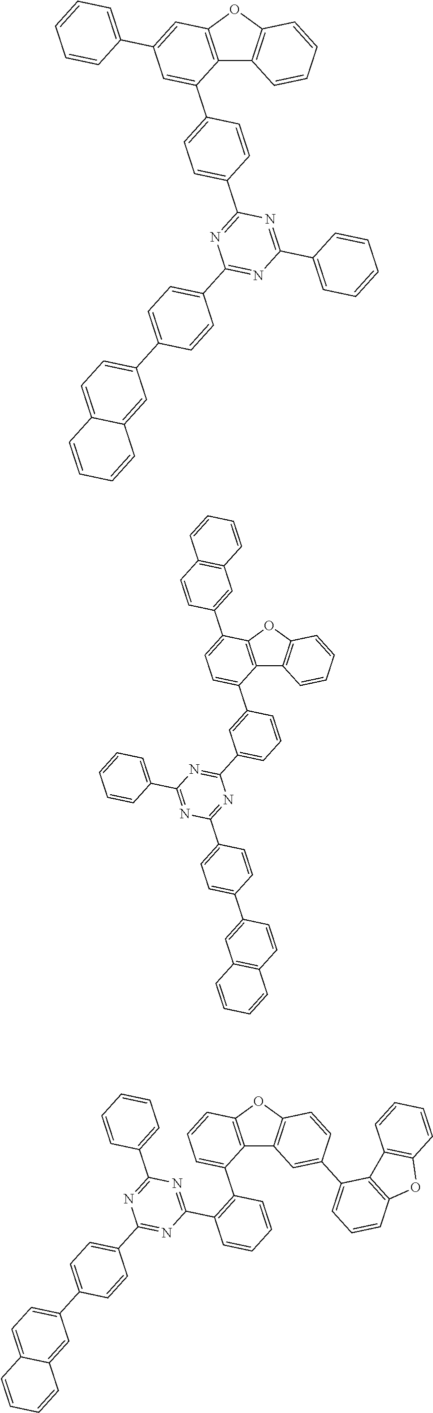 [SMILES: c1ccc(-c2cc(-c3ccc(-c4nc(-c5ccccc5)nc(-c5ccc(-c6ccc7ccccc7c6)cc5)n4)cc3)c3c(c2)oc2ccccc23)cc1 ; c1ccc(-c2nc(-c3ccc(-c4ccc5ccccc5c4)cc3)nc(-c3cccc(-c4ccc(-c5ccc6ccccc6c5)c5oc6ccccc6c45)c3)n2)cc1 ; c1ccc(-c2nc(-c3ccc(-c4ccc5ccccc5c4)cc3)nc(-c3ccccc3-c3cccc4oc5ccc(-c6cccc7oc8ccccc8c67)cc5c34)n2)cc1]

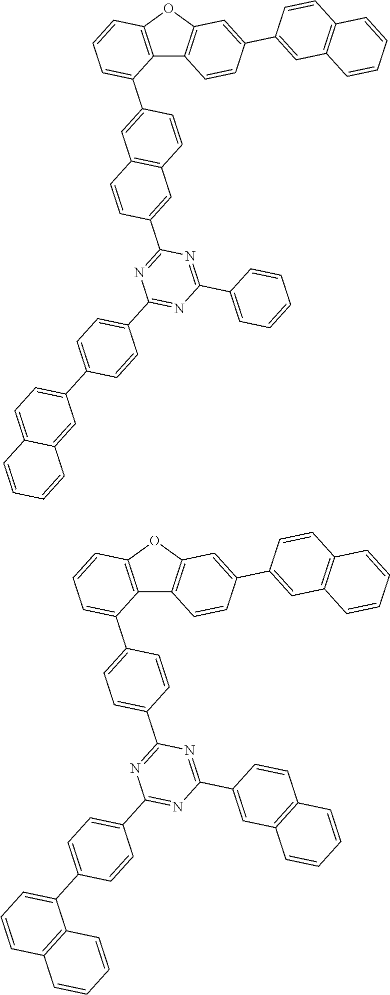 [SMILES: c1ccc(-c2nc(-c3ccc(-c4ccc5ccccc5c4)cc3)nc(-c3ccc4cc(-c5cccc6oc7cc(-c8ccc9ccccc9c8)ccc7c56)ccc4c3)n2)cc1 ; c1ccc2cc(-c3ccc4c(c3)oc3cccc(-c5ccc(-c6nc(-c7ccc(-c8cccc9ccccc89)cc7)nc(-c7ccc8ccccc8c7)n6)cc5)c34)ccc2c1]

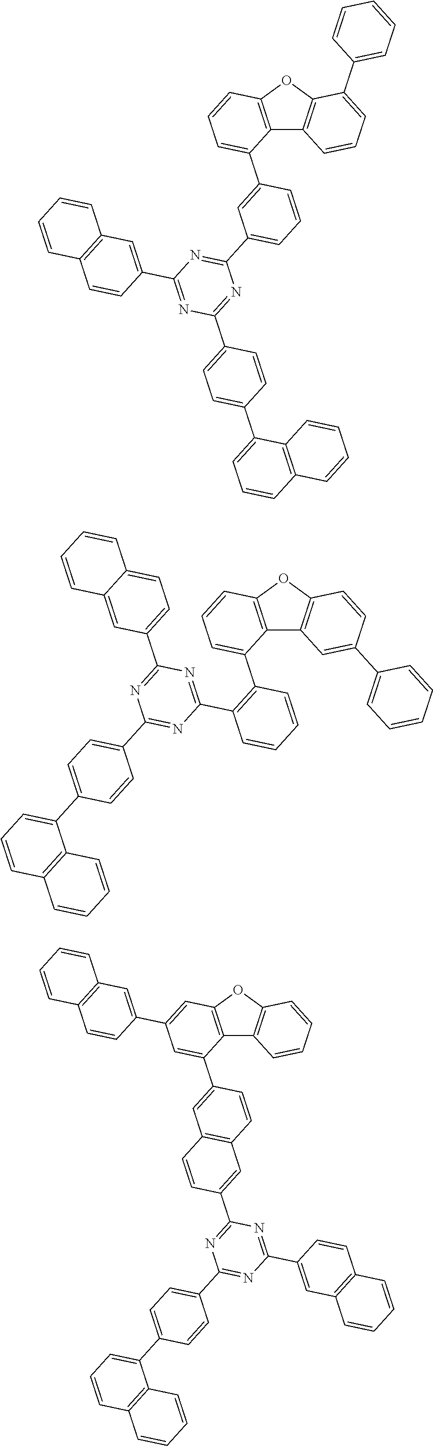

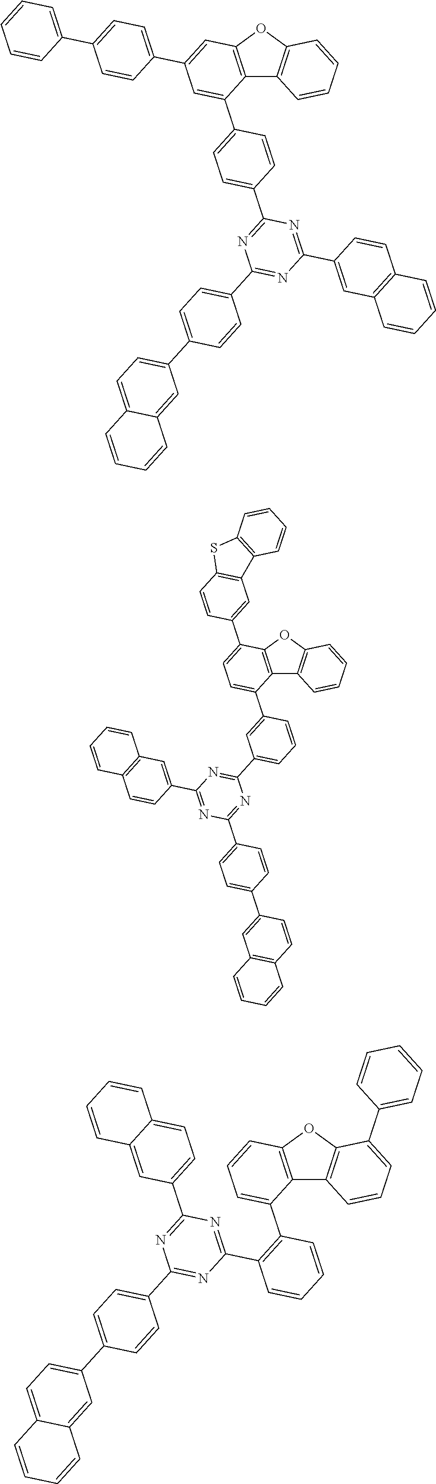 [SMILES: c1ccc(-c2ccc(-c3cc(-c4ccc(-c5nc(-c6ccc(-c7ccc8ccccc8c7)cc6)nc(-c6ccc7ccccc7c6)n5)cc4)c4c(c3)oc3ccccc34)cc2)cc1 ; c1cc(-c2nc(-c3ccc(-c4ccc5ccccc5c4)cc3)nc(-c3ccc4ccccc4c3)n2)cc(-c2ccc(-c3ccc4sc5ccccc5c4c3)c3oc4ccccc4c23)c1 ; c1ccc(-c2cccc3c2oc2cccc(-c4ccccc4-c4nc(-c5ccc(-c6ccc7ccccc7c6)cc5)nc(-c5ccc6ccccc6c5)n4)c23)cc1]

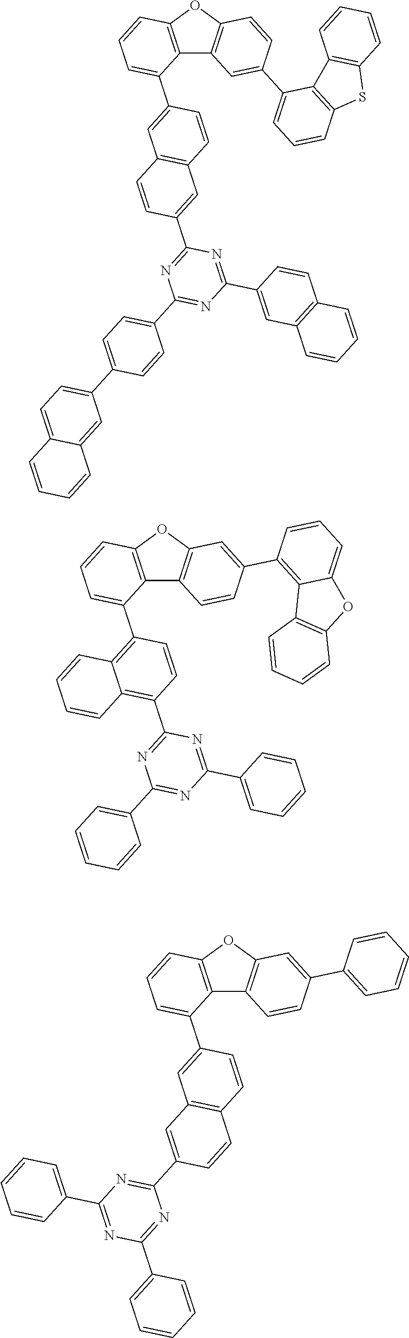 [SMILES: c1ccc2cc(-c3ccc(-c4nc(-c5ccc6ccccc6c5)nc(-c5ccc6cc(-c7cccc8oc9ccc(-c%10cccc%11sc%12ccccc%12c%10%11)cc9c78)ccc6c5)n4)cc3)ccc2c1 ; c1ccc(-c2nc(-c3ccccc3)nc(-c3ccc(-c4cccc5oc6cc(-c7cccc8oc9ccccc9c78)ccc6c45)c4ccccc34)n2)cc1 ; c1ccc(-c2ccc3c(c2)oc2cccc(-c4ccc5ccc(-c6nc(-c7ccccc7)nc(-c7ccccc7)n6)cc5c4)c23)cc1]

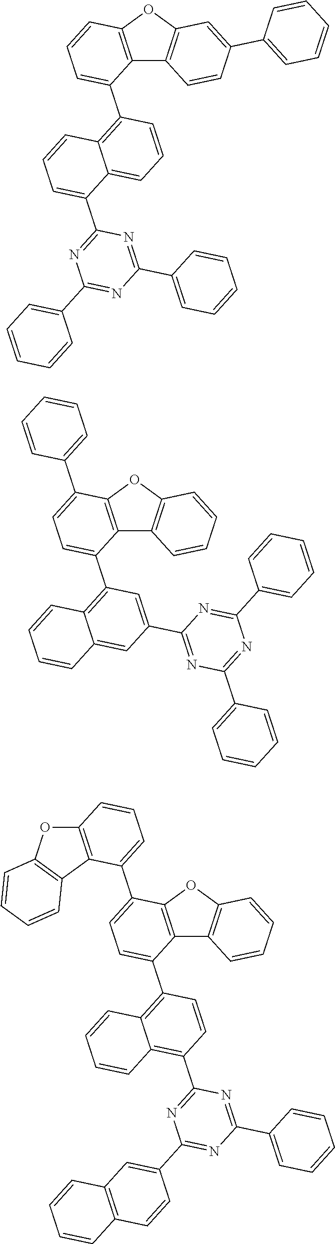

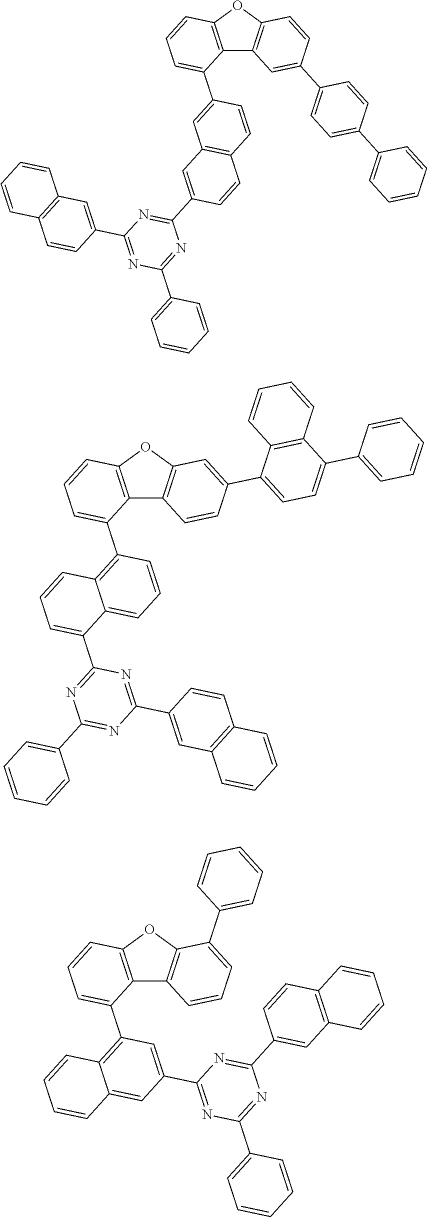 [SMILES: c1ccc(-c2ccc(-c3ccc4oc5cccc(-c6ccc7ccc(-c8nc(-c9ccccc9)nc(-c9ccc%10ccccc%10c9)n8)cc7c6)c5c4c3)cc2)cc1 ; c1ccc(-c2nc(-c3ccc4ccccc4c3)nc(-c3cccc4c(-c5cccc6oc7cc(-c8ccc(-c9ccccc9)c9ccccc89)ccc7c56)cccc34)n2)cc1 ; c1ccc(-c2nc(-c3ccc4ccccc4c3)nc(-c3cc(-c4cccc5oc6c(-c7ccccc7)cccc6c45)c4ccccc4c3)n2)cc1]

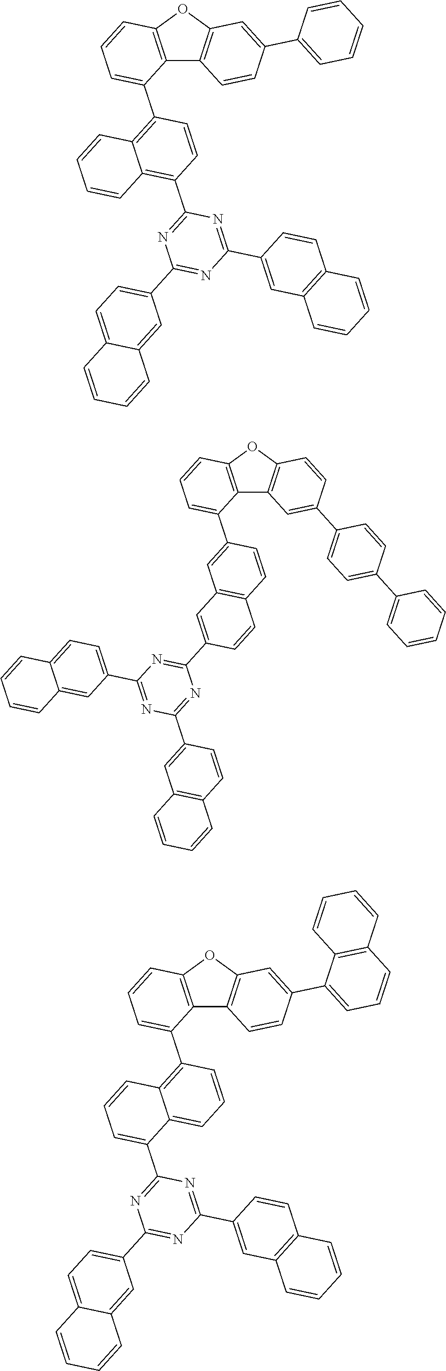 [SMILES: c1ccc(-c2ccc3c(c2)oc2cccc(-c4ccc(-c5nc(-c6ccc7ccccc7c6)nc(-c6ccc7ccccc7c6)n5)c5ccccc45)c23)cc1 ; c1ccc(-c2ccc(-c3ccc4oc5cccc(-c6ccc7ccc(-c8nc(-c9ccc%10ccccc%10c9)nc(-c9ccc%10ccccc%10c9)n8)cc7c6)c5c4c3)cc2)cc1 ; c1ccc2cc(-c3nc(-c4ccc5ccccc5c4)nc(-c4cccc5c(-c6cccc7oc8cc(-c9cccc%10ccccc9%10)ccc8c67)cccc45)n3)ccc2c1]

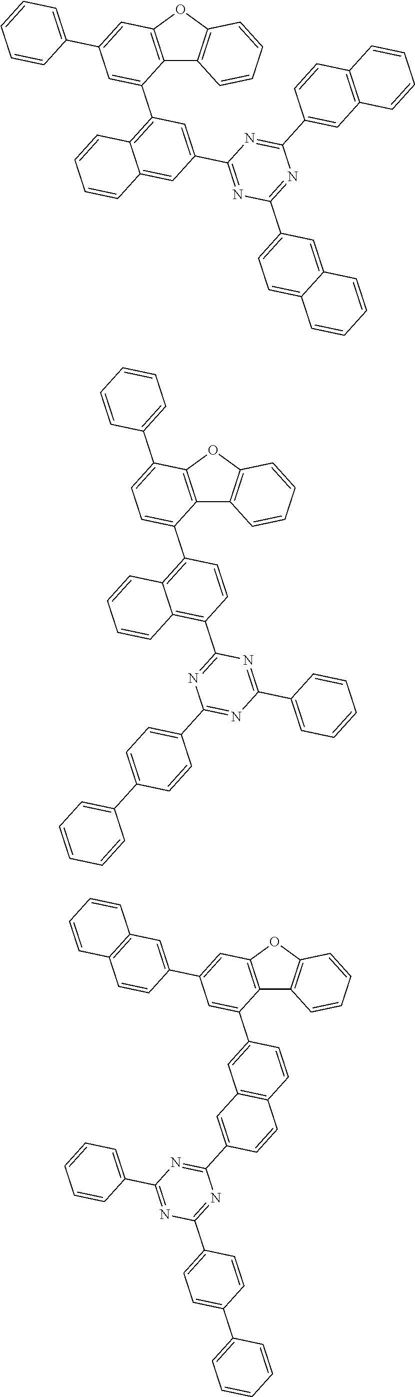

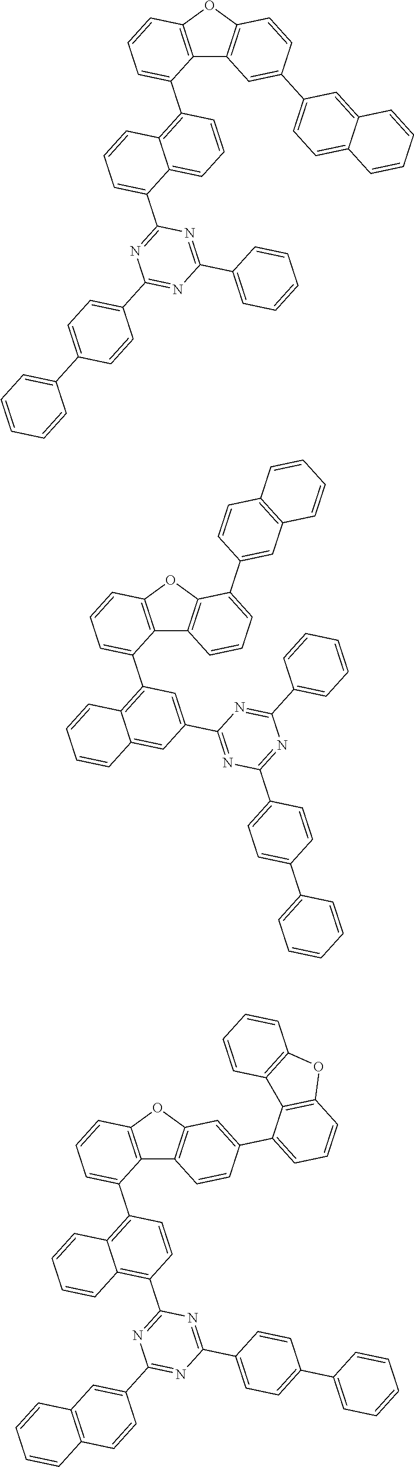 [SMILES: c1ccc(-c2ccc(-c3nc(-c4ccccc4)nc(-c4cccc5c(-c6cccc7oc8ccc(-c9ccc%10ccccc%10c9)cc8c67)cccc45)n3)cc2)cc1 ; c1ccc(-c2ccc(-c3nc(-c4ccccc4)nc(-c4cc(-c5cccc6oc7c(-c8ccc9ccccc9c8)cccc7c56)c5ccccc5c4)n3)cc2)cc1 ; c1ccc(-c2ccc(-c3nc(-c4ccc5ccccc5c4)nc(-c4ccc(-c5cccc6oc7cc(-c8cccc9oc%10ccccc%10c89)ccc7c56)c5ccccc45)n3)cc2)cc1]

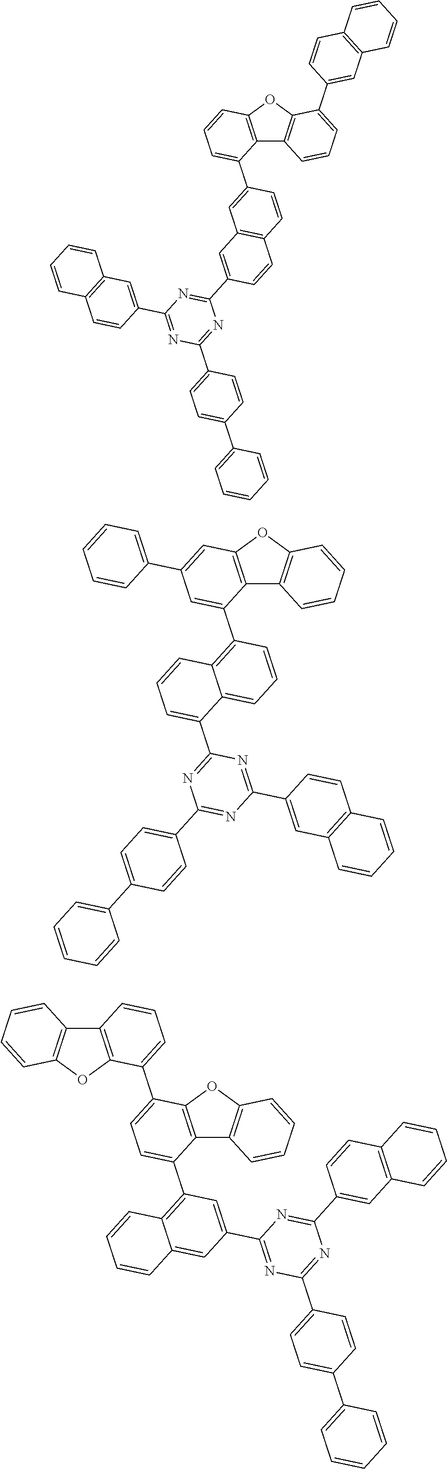

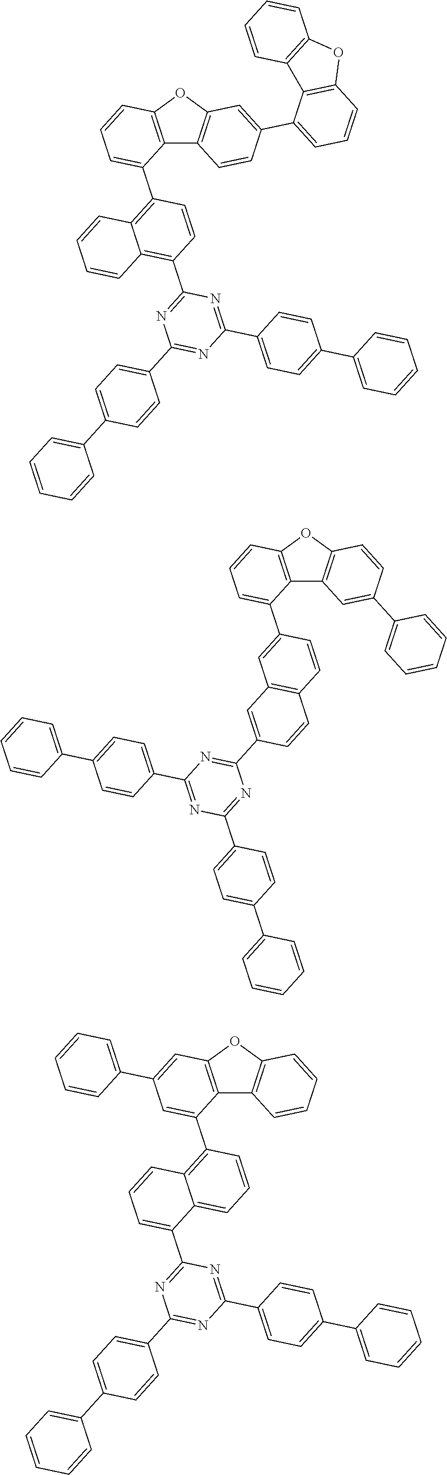 [SMILES: c1ccc(-c2ccc(-c3nc(-c4ccc(-c5ccccc5)cc4)nc(-c4ccc(-c5cccc6oc7cc(-c8cccc9oc%10ccccc%10c89)ccc7c56)c5ccccc45)n3)cc2)cc1 ; c1ccc(-c2ccc(-c3nc(-c4ccc(-c5ccccc5)cc4)nc(-c4ccc5ccc(-c6cccc7oc8ccc(-c9ccccc9)cc8c67)cc5c4)n3)cc2)cc1 ; c1ccc(-c2ccc(-c3nc(-c4ccc(-c5ccccc5)cc4)nc(-c4cccc5c(-c6cc(-c7ccccc7)cc7oc8ccccc8c67)cccc45)n3)cc2)cc1]

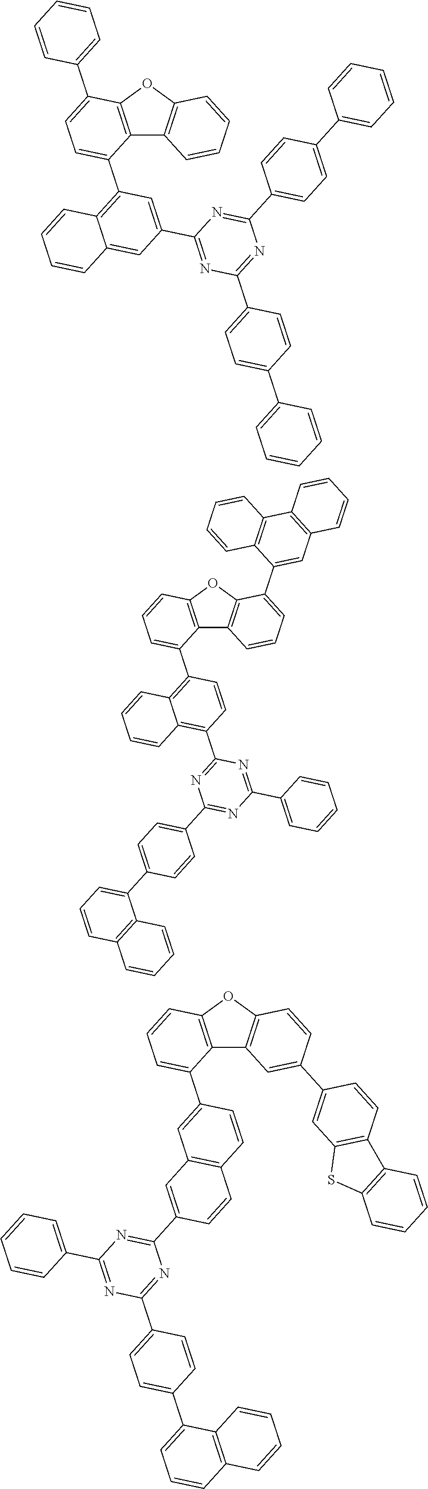

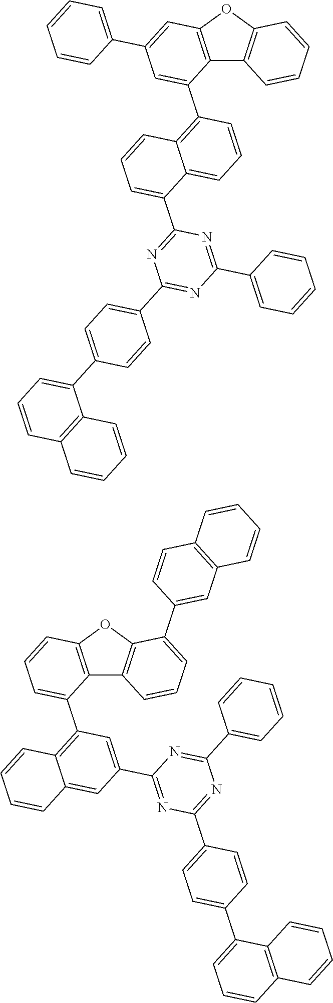 [SMILES: c1ccc(-c2cc(-c3cccc4c(-c5nc(-c6ccccc6)nc(-c6ccc(-c7cccc8ccccc78)cc6)n5)cccc34)c3c(c2)oc2ccccc23)cc1 ; c1ccc(-c2nc(-c3ccc(-c4cccc5ccccc45)cc3)nc(-c3cc(-c4cccc5oc6c(-c7ccc8ccccc8c7)cccc6c45)c4ccccc4c3)n2)cc1]

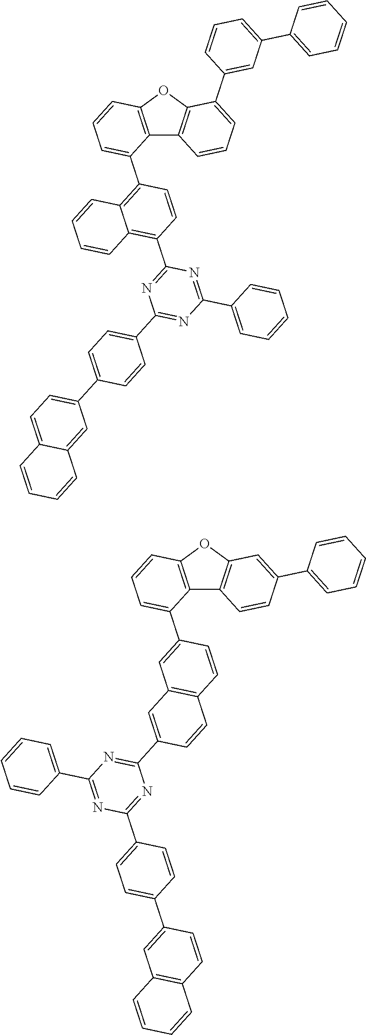 [SMILES: c1ccc(-c2cccc(-c3cccc4c3oc3cccc(-c5ccc(-c6nc(-c7ccccc7)nc(-c7ccc(-c8ccc9ccccc9c8)cc7)n6)c6ccccc56)c34)c2)cc1 ; c1ccc(-c2ccc3c(c2)oc2cccc(-c4ccc5ccc(-c6nc(-c7ccccc7)nc(-c7ccc(-c8ccc9ccccc9c8)cc7)n6)cc5c4)c23)cc1]

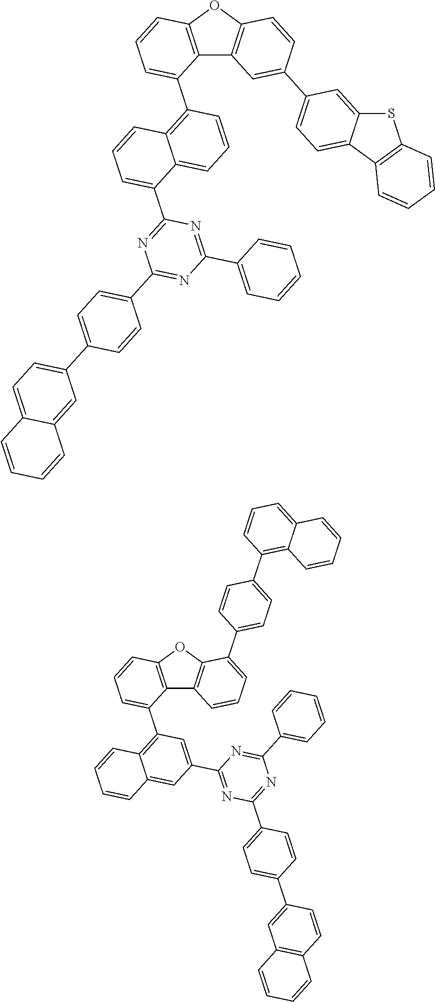 [SMILES: c1ccc(-c2nc(-c3ccc(-c4ccc5ccccc5c4)cc3)nc(-c3cccc4c(-c5cccc6oc7ccc(-c8ccc9c(c8)sc8ccccc89)cc7c56)cccc34)n2)cc1 ; c1ccc(-c2nc(-c3ccc(-c4ccc5ccccc5c4)cc3)nc(-c3cc(-c4cccc5oc6c(-c7ccc(-c8cccc9ccccc89)cc7)cccc6c45)c4ccccc4c3)n2)cc1]

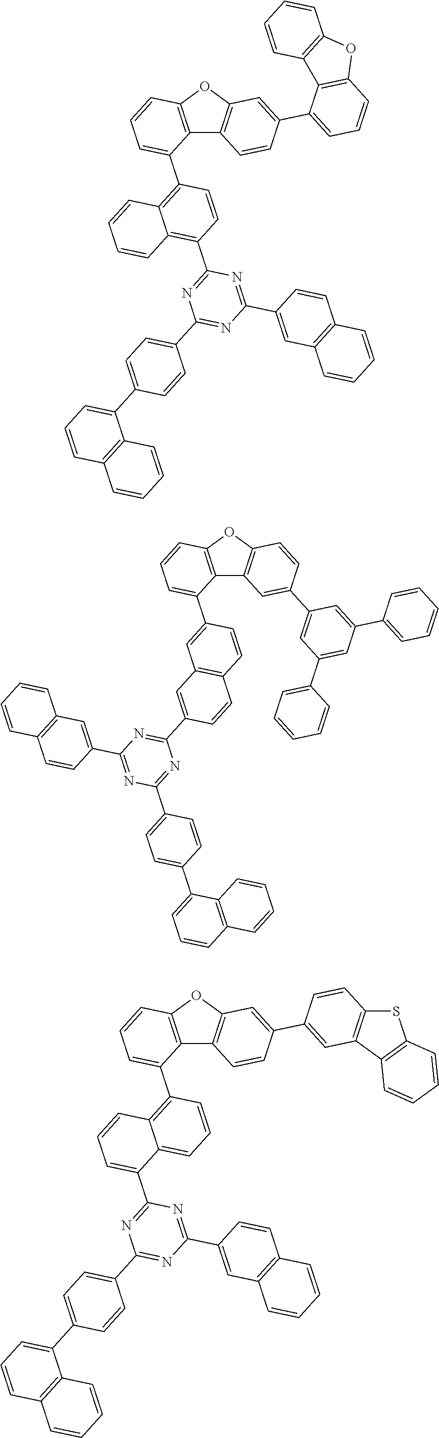 [SMILES: c1ccc2cc(-c3nc(-c4ccc(-c5cccc6ccccc56)cc4)nc(-c4ccc(-c5cccc6oc7cc(-c8cccc9oc%10ccccc%10c89)ccc7c56)c5ccccc45)n3)ccc2c1 ; c1ccc(-c2cc(-c3ccccc3)cc(-c3ccc4oc5cccc(-c6ccc7ccc(-c8nc(-c9ccc(-c%10cccc%11ccccc%10%11)cc9)nc(-c9ccc%10ccccc%10c9)n8)cc7c6)c5c4c3)c2)cc1 ; c1ccc2cc(-c3nc(-c4ccc(-c5cccc6ccccc56)cc4)nc(-c4cccc5c(-c6cccc7oc8cc(-c9ccc%10sc%11ccccc%11c%10c9)ccc8c67)cccc45)n3)ccc2c1]

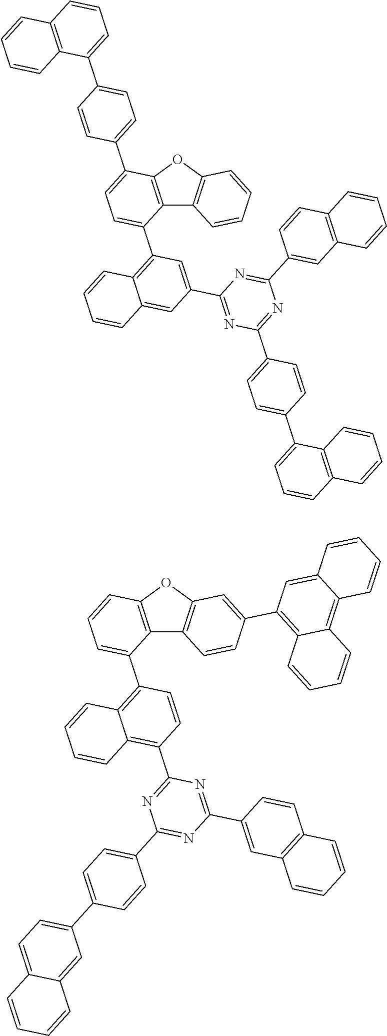 [SMILES: c1ccc2cc(-c3nc(-c4ccc(-c5cccc6ccccc56)cc4)nc(-c4cc(-c5ccc(-c6ccc(-c7cccc8ccccc78)cc6)c6oc7ccccc7c56)c5ccccc5c4)n3)ccc2c1 ; c1ccc2cc(-c3ccc(-c4nc(-c5ccc6ccccc6c5)nc(-c5ccc(-c6cccc7oc8cc(-c9cc%10ccccc%10c%10ccccc9%10)ccc8c67)c6ccccc56)n4)cc3)ccc2c1]

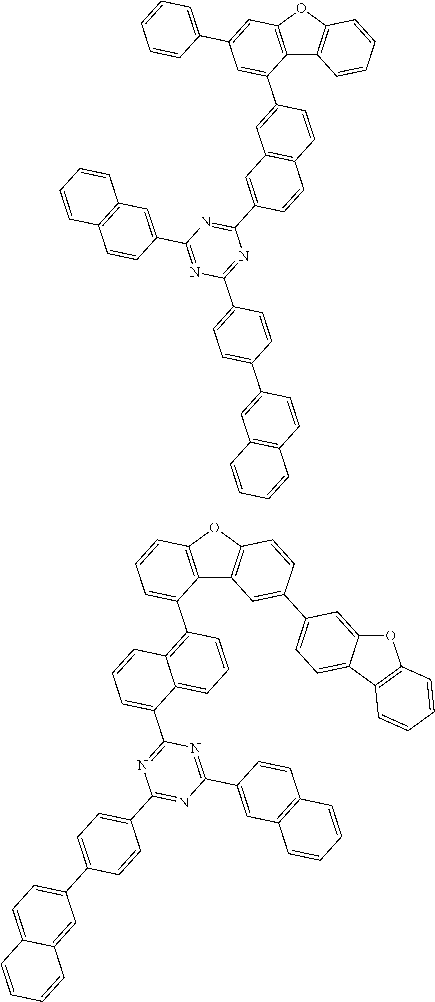

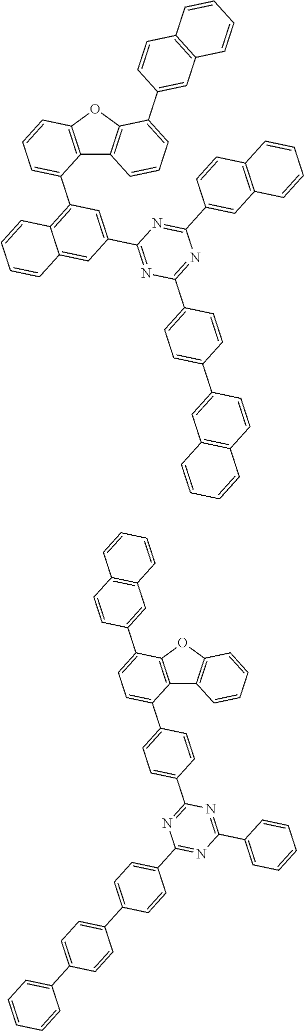 [SMILES: c1ccc2cc(-c3ccc(-c4nc(-c5ccc6ccccc6c5)nc(-c5cc(-c6cccc7oc8c(-c9ccc%10ccccc%10c9)cccc8c67)c6ccccc6c5)n4)cc3)ccc2c1 ; c1ccc(-c2ccc(-c3ccc(-c4nc(-c5ccccc5)nc(-c5ccc(-c6ccc(-c7ccc8ccccc8c7)c7oc8ccccc8c67)cc5)n4)cc3)cc2)cc1]

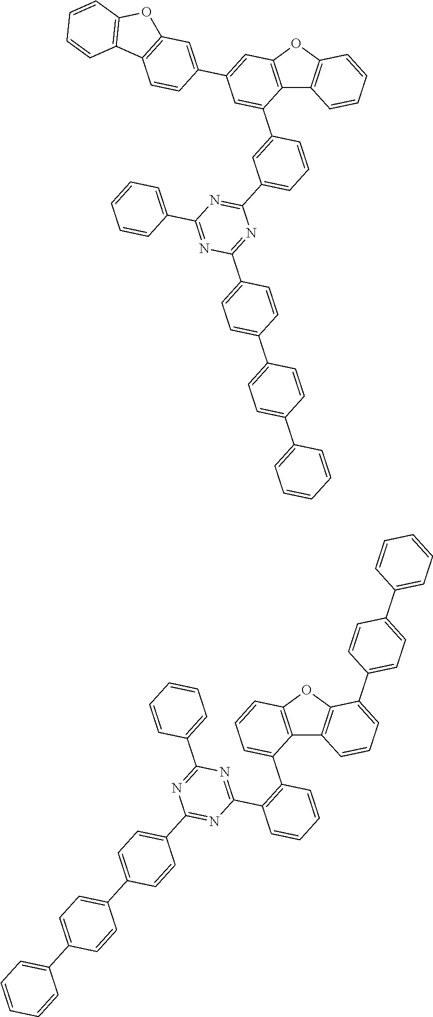 [SMILES: c1ccc(-c2ccc(-c3ccc(-c4nc(-c5ccccc5)nc(-c5cccc(-c6cc(-c7ccc8c(c7)oc7ccccc78)cc7oc8ccccc8c67)c5)n4)cc3)cc2)cc1 ; c1ccc(-c2ccc(-c3ccc(-c4nc(-c5ccccc5)nc(-c5ccccc5-c5cccc6oc7c(-c8ccc(-c9ccccc9)cc8)cccc7c56)n4)cc3)cc2)cc1]

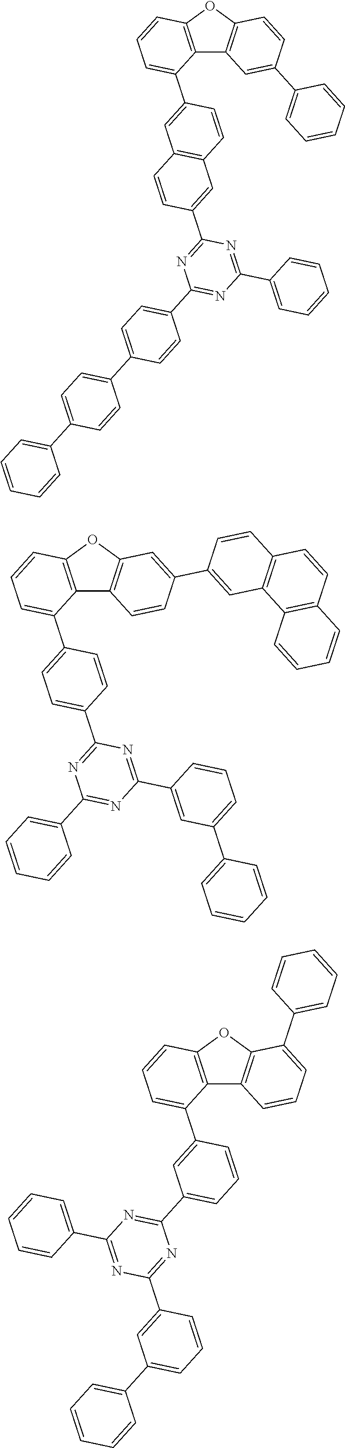 [SMILES: c1ccc(-c2ccc(-c3ccc(-c4nc(-c5ccccc5)nc(-c5ccc6cc(-c7cccc8oc9ccc(-c%10ccccc%10)cc9c78)ccc6c5)n4)cc3)cc2)cc1 ; c1ccc(-c2cccc(-c3nc(-c4ccccc4)nc(-c4ccc(-c5cccc6oc7cc(-c8ccc9ccc%10ccccc%10c9c8)ccc7c56)cc4)n3)c2)cc1 ; c1ccc(-c2cccc(-c3nc(-c4ccccc4)nc(-c4cccc(-c5cccc6oc7c(-c8ccccc8)cccc7c56)c4)n3)c2)cc1]

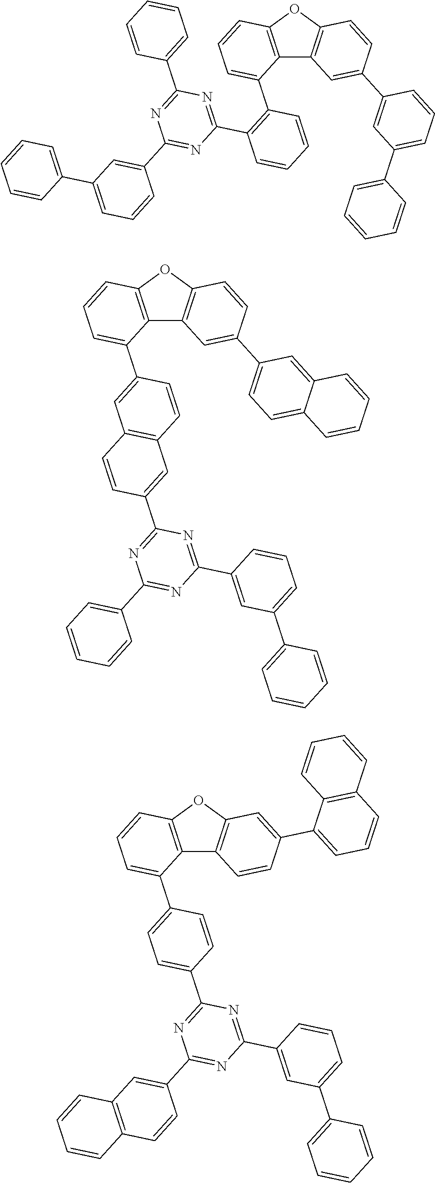

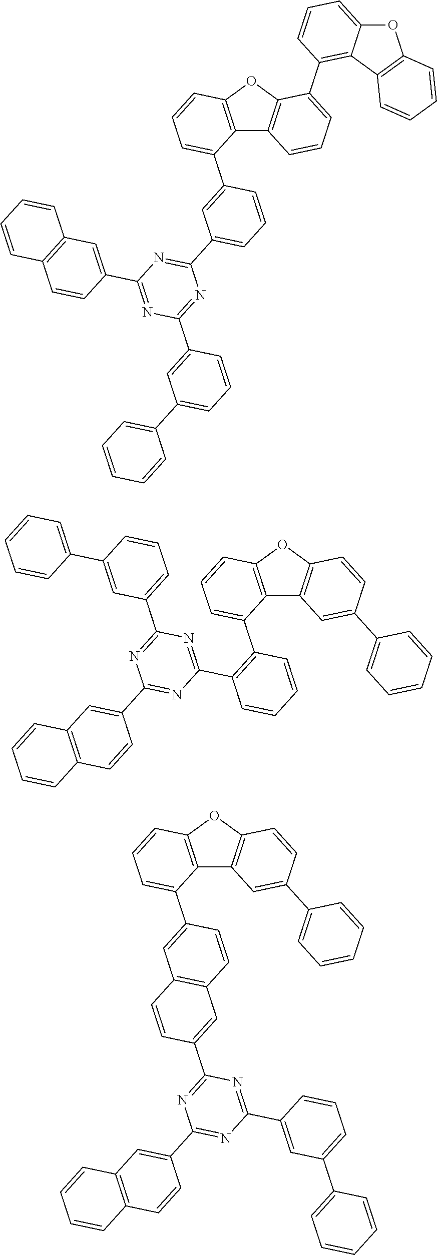 [SMILES: c1ccc(-c2cccc(-c3nc(-c4cccc(-c5cccc6oc7c(-c8cccc9oc%10ccccc%10c89)cccc7c56)c4)nc(-c4ccc5ccccc5c4)n3)c2)cc1 ; c1ccc(-c2cccc(-c3nc(-c4ccc5ccccc5c4)nc(-c4ccccc4-c4cccc5oc6ccc(-c7ccccc7)cc6c45)n3)c2)cc1 ; c1ccc(-c2cccc(-c3nc(-c4ccc5ccccc5c4)nc(-c4ccc5cc(-c6cccc7oc8ccc(-c9ccccc9)cc8c67)ccc5c4)n3)c2)cc1]

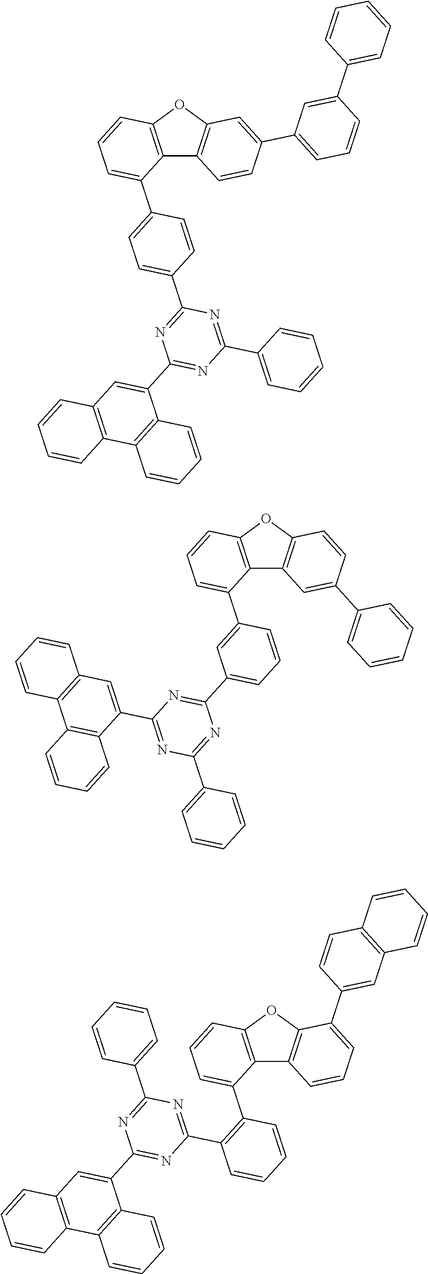 [SMILES: c1ccc(-c2cccc(-c3ccc4c(c3)oc3cccc(-c5ccc(-c6nc(-c7ccccc7)nc(-c7cc8ccccc8c8ccccc78)n6)cc5)c34)c2)cc1 ; c1ccc(-c2ccc3oc4cccc(-c5cccc(-c6nc(-c7ccccc7)nc(-c7cc8ccccc8c8ccccc78)n6)c5)c4c3c2)cc1 ; c1ccc(-c2nc(-c3ccccc3-c3cccc4oc5c(-c6ccc7ccccc7c6)cccc5c34)nc(-c3cc4ccccc4c4ccccc34)n2)cc1]

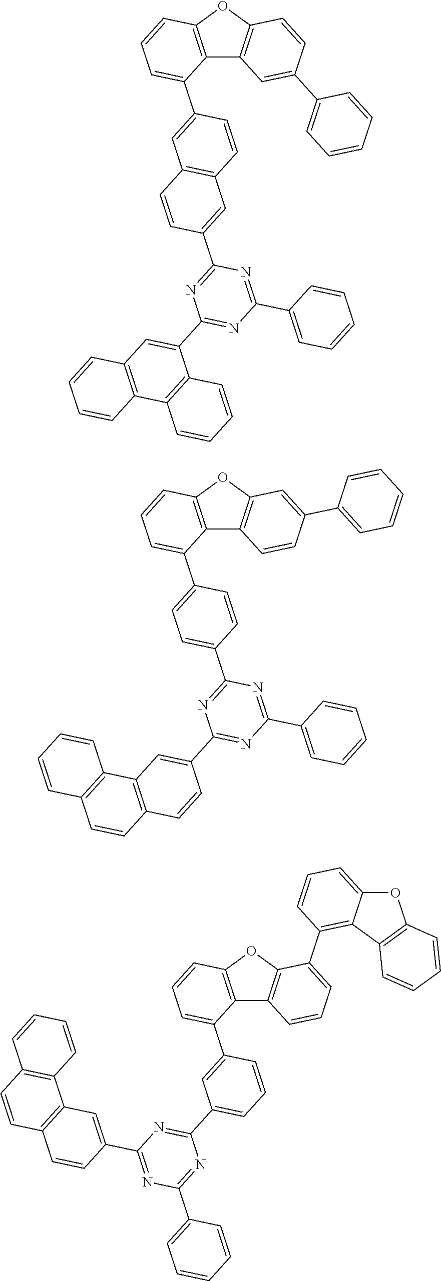

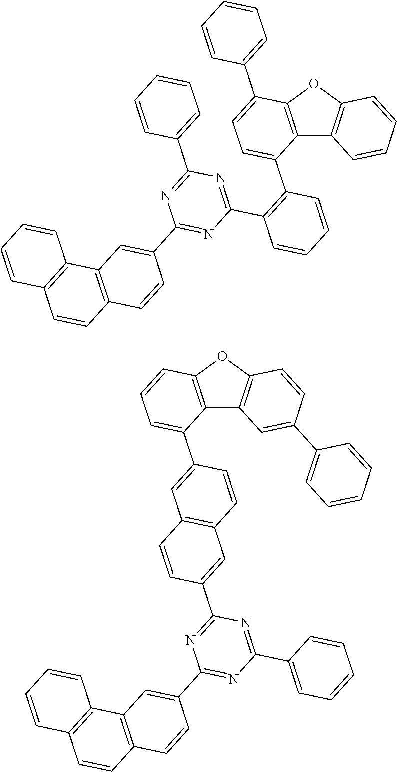 [SMILES: c1ccc(-c2nc(-c3ccc4ccc5ccccc5c4c3)nc(-c3ccccc3-c3ccc(-c4ccccc4)c4oc5ccccc5c34)n2)cc1 ; c1ccc(-c2ccc3oc4cccc(-c5ccc6cc(-c7nc(-c8ccccc8)nc(-c8ccc9ccc%10ccccc%10c9c8)n7)ccc6c5)c4c3c2)cc1]

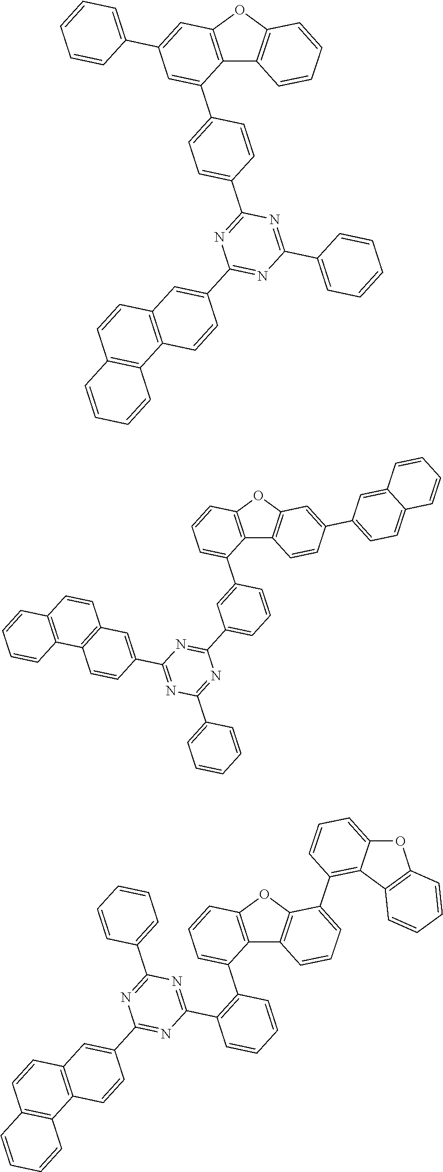 [SMILES: c1ccc(-c2cc(-c3ccc(-c4nc(-c5ccccc5)nc(-c5ccc6c(ccc7ccccc76)c5)n4)cc3)c3c(c2)oc2ccccc23)cc1 ; c1ccc(-c2nc(-c3cccc(-c4cccc5oc6cc(-c7ccc8ccccc8c7)ccc6c45)c3)nc(-c3ccc4c(ccc5ccccc54)c3)n2)cc1 ; c1ccc(-c2nc(-c3ccc4c(ccc5ccccc54)c3)nc(-c3ccccc3-c3cccc4oc5c(-c6cccc7oc8ccccc8c67)cccc5c34)n2)cc1]

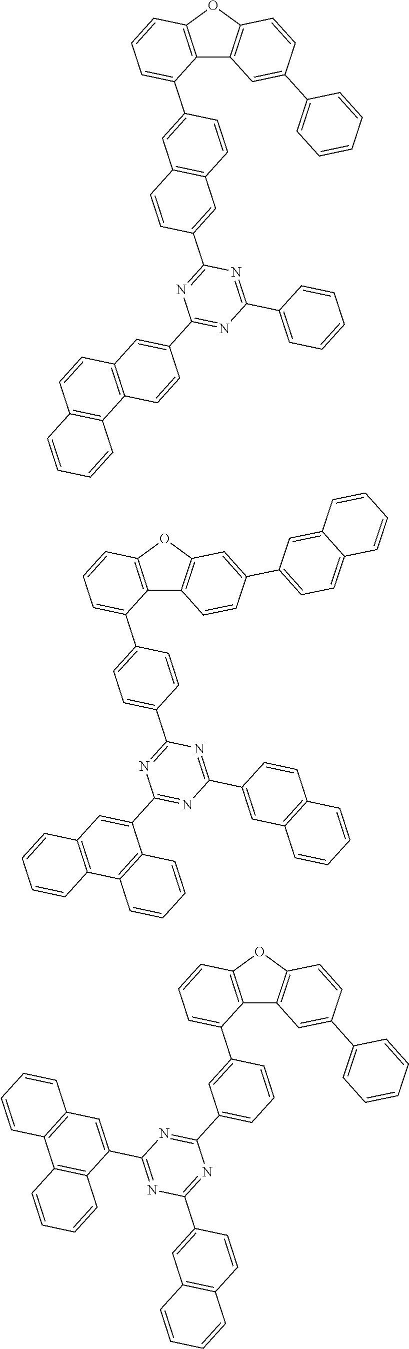 [SMILES: c1ccc(-c2ccc3oc4cccc(-c5ccc6cc(-c7nc(-c8ccccc8)nc(-c8ccc9c(ccc%10ccccc%109)c8)n7)ccc6c5)c4c3c2)cc1 ; c1ccc2cc(-c3ccc4c(c3)oc3cccc(-c5ccc(-c6nc(-c7ccc8ccccc8c7)nc(-c7cc8ccccc8c8ccccc78)n6)cc5)c34)ccc2c1 ; c1ccc(-c2ccc3oc4cccc(-c5cccc(-c6nc(-c7ccc8ccccc8c7)nc(-c7cc8ccccc8c8ccccc78)n6)c5)c4c3c2)cc1]

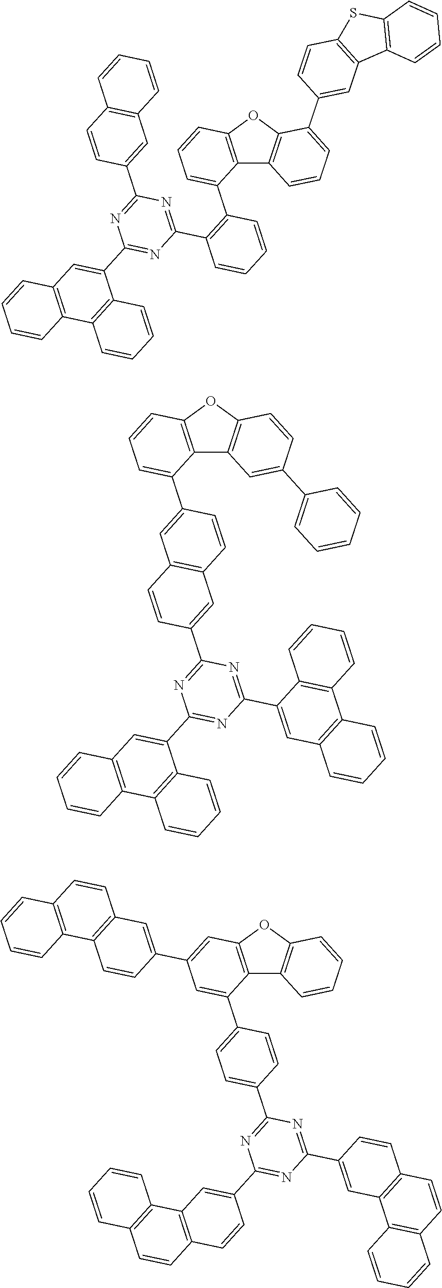 [SMILES: c1ccc(-c2cccc3oc4c(-c5ccc6sc7ccccc7c6c5)cccc4c23)c(-c2nc(-c3ccc4ccccc4c3)nc(-c3cc4ccccc4c4ccccc34)n2)c1 ; c1ccc(-c2ccc3oc4cccc(-c5ccc6cc(-c7nc(-c8cc9ccccc9c9ccccc89)nc(-c8cc9ccccc9c9ccccc89)n7)ccc6c5)c4c3c2)cc1 ; c1ccc2c(c1)ccc1cc(-c3cc(-c4ccc(-c5nc(-c6ccc7ccc8ccccc8c7c6)nc(-c6ccc7ccc8ccccc8c7c6)n5)cc4)c4c(c3)oc3ccccc34)ccc12]

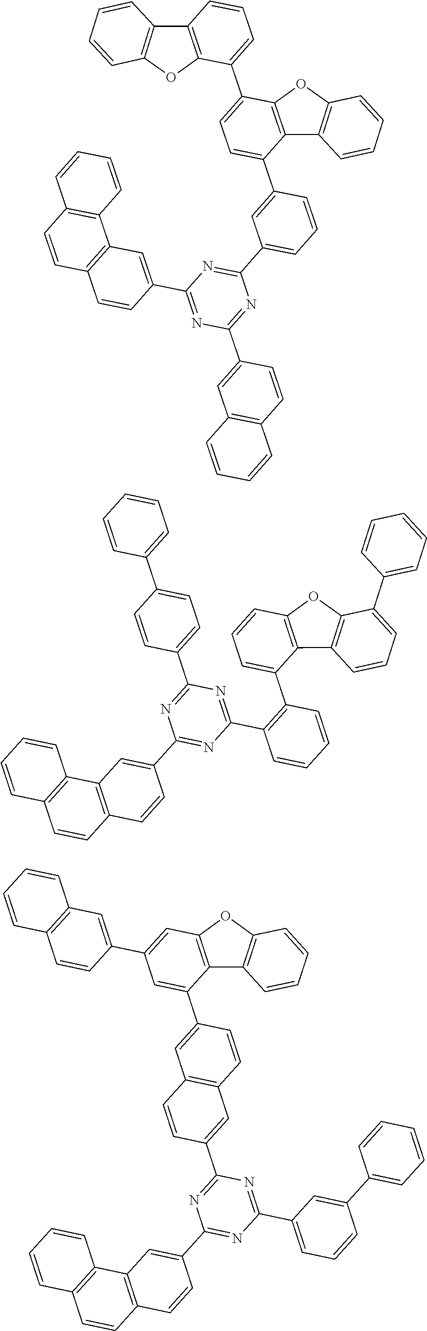

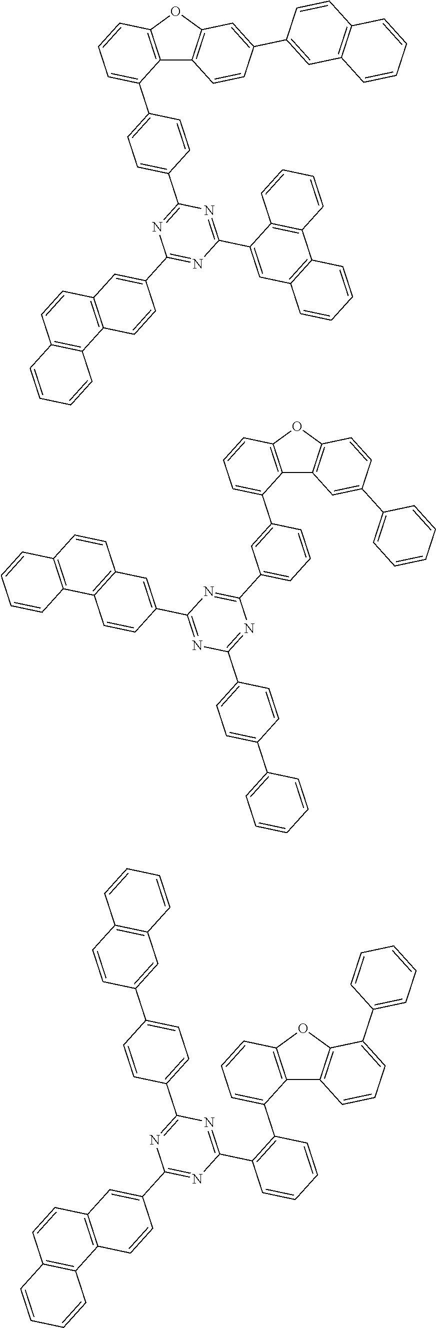 [SMILES: c1ccc2cc(-c3ccc4c(c3)oc3cccc(-c5ccc(-c6nc(-c7ccc8c(ccc9ccccc98)c7)nc(-c7cc8ccccc8c8ccccc78)n6)cc5)c34)ccc2c1 ; c1ccc(-c2ccc(-c3nc(-c4cccc(-c5cccc6oc7ccc(-c8ccccc8)cc7c56)c4)nc(-c4ccc5c(ccc6ccccc65)c4)n3)cc2)cc1 ; c1ccc(-c2cccc3c2oc2cccc(-c4ccccc4-c4nc(-c5ccc(-c6ccc7ccccc7c6)cc5)nc(-c5ccc6c(ccc7ccccc76)c5)n4)c23)cc1]

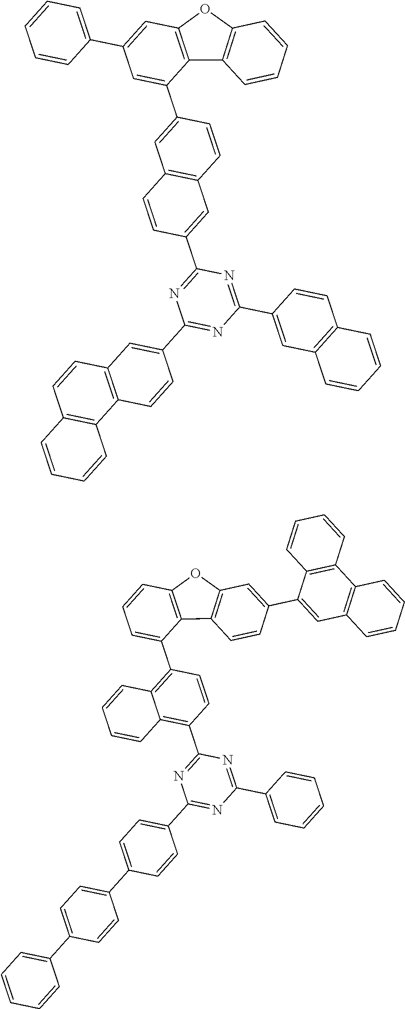 [SMILES: c1ccc(-c2cc(-c3ccc4cc(-c5nc(-c6ccc7ccccc7c6)nc(-c6ccc7c(ccc8ccccc87)c6)n5)ccc4c3)c3c(c2)oc2ccccc23)cc1 ; c1ccc(-c2ccc(-c3ccc(-c4nc(-c5ccccc5)nc(-c5ccc(-c6cccc7oc8cc(-c9cc%10ccccc%10c%10ccccc9%10)ccc8c67)c6ccccc56)n4)cc3)cc2)cc1]

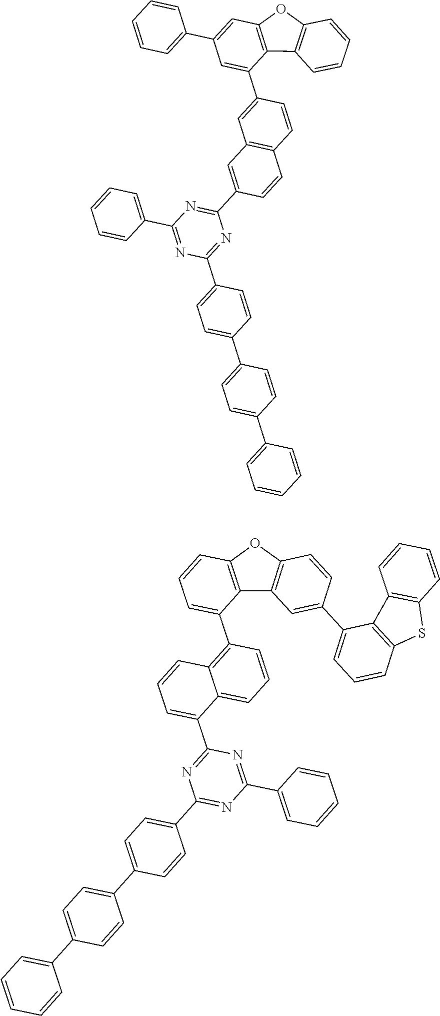 [SMILES: c1ccc(-c2ccc(-c3ccc(-c4nc(-c5ccccc5)nc(-c5ccc6ccc(-c7cc(-c8ccccc8)cc8oc9ccccc9c78)cc6c5)n4)cc3)cc2)cc1 ; c1ccc(-c2ccc(-c3ccc(-c4nc(-c5ccccc5)nc(-c5cccc6c(-c7cccc8oc9ccc(-c%10cccc%11sc%12ccccc%12c%10%11)cc9c78)cccc56)n4)cc3)cc2)cc1]

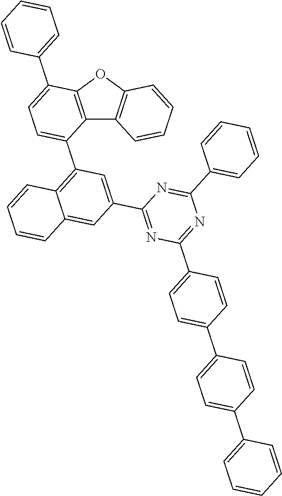 [SMILES: c1ccc(-c2ccc(-c3ccc(-c4nc(-c5ccccc5)nc(-c5cc(-c6ccc(-c7ccccc7)c7oc8ccccc8c67)c6ccccc6c5)n4)cc3)cc2)cc1]

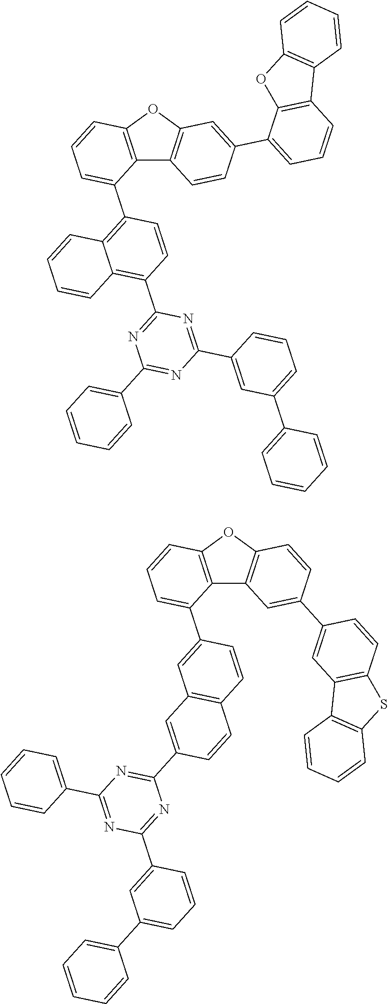 [SMILES: c1ccc(-c2cccc(-c3nc(-c4ccccc4)nc(-c4ccc(-c5cccc6oc7cc(-c8cccc9c8oc8ccccc89)ccc7c56)c5ccccc45)n3)c2)cc1 ; c1ccc(-c2cccc(-c3nc(-c4ccccc4)nc(-c4ccc5ccc(-c6cccc7oc8ccc(-c9ccc%10sc%11ccccc%11c%10c9)cc8c67)cc5c4)n3)c2)cc1]

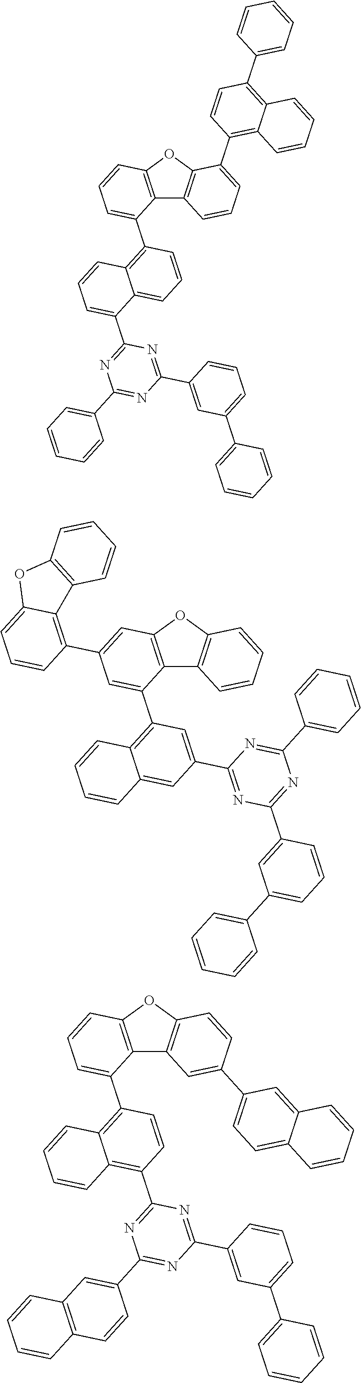

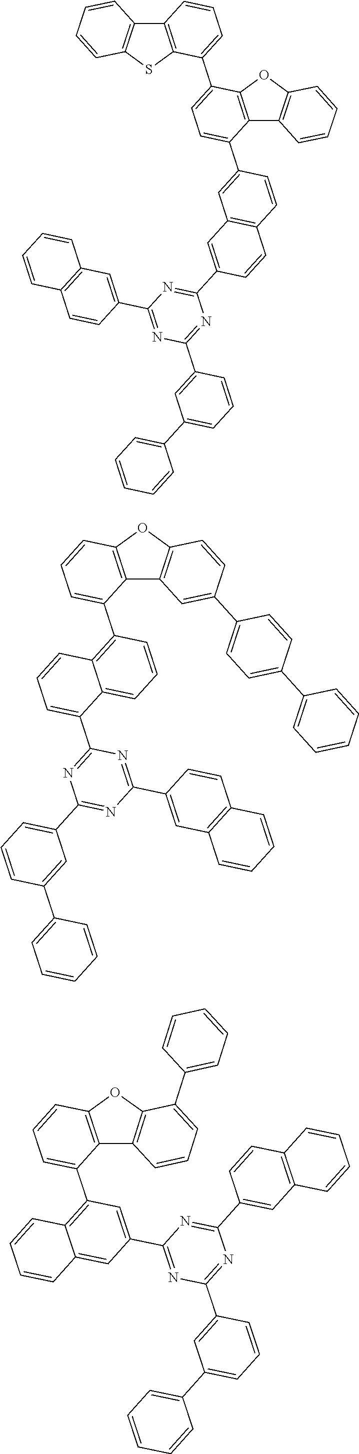

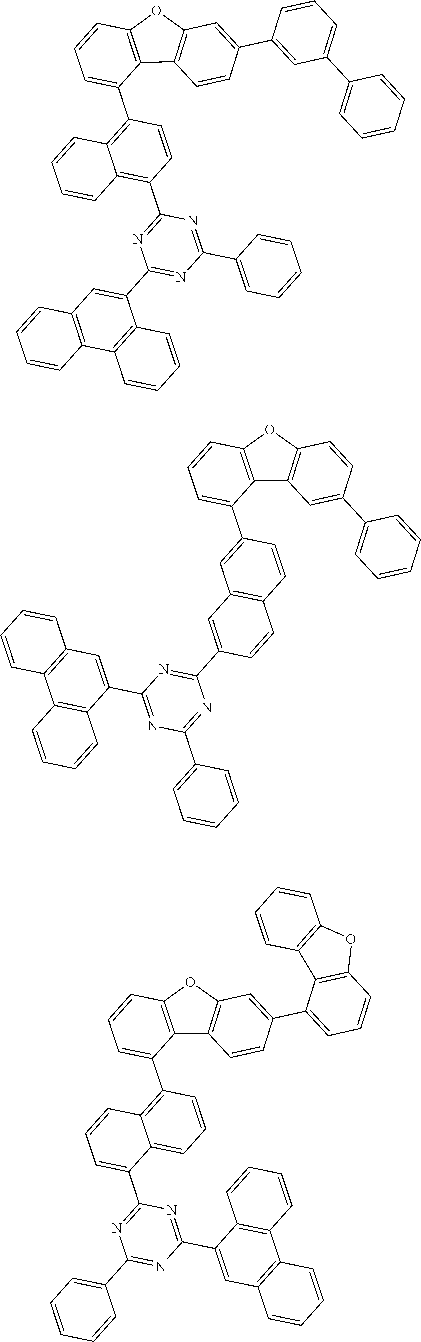 [SMILES: c1ccc(-c2cccc(-c3ccc4c(c3)oc3cccc(-c5ccc(-c6nc(-c7ccccc7)nc(-c7cc8ccccc8c8ccccc78)n6)c6ccccc56)c34)c2)cc1 ; c1ccc(-c2ccc3oc4cccc(-c5ccc6ccc(-c7nc(-c8ccccc8)nc(-c8cc9ccccc9c9ccccc89)n7)cc6c5)c4c3c2)cc1 ; c1ccc(-c2nc(-c3cccc4c(-c5cccc6oc7cc(-c8cccc9oc%10ccccc%10c89)ccc7c56)cccc34)nc(-c3cc4ccccc4c4ccccc34)n2)cc1]

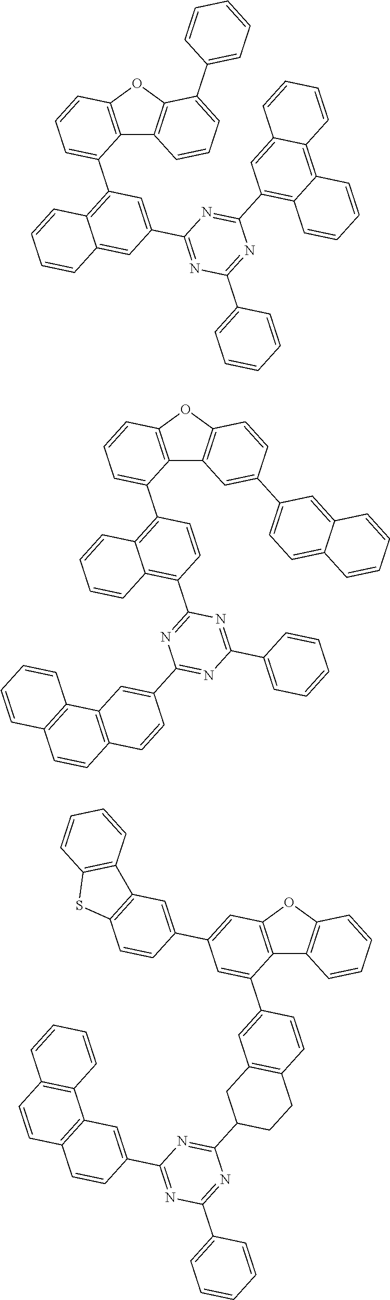

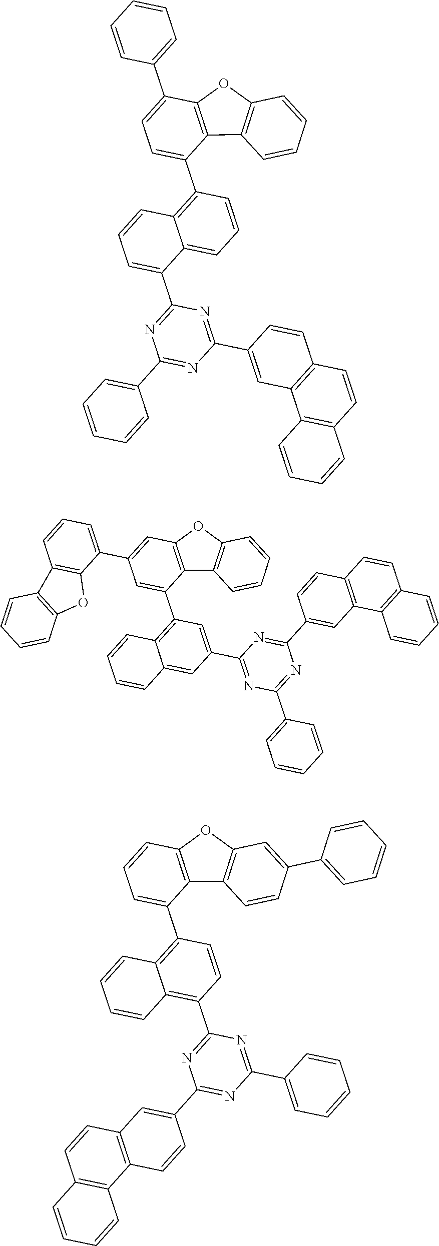 [SMILES: c1ccc(-c2nc(-c3ccc4ccc5ccccc5c4c3)nc(-c3cccc4c(-c5ccc(-c6ccccc6)c6oc7ccccc7c56)cccc34)n2)cc1 ; c1ccc(-c2nc(-c3cc(-c4cc(-c5cccc6c5oc5ccccc56)cc5oc6ccccc6c45)c4ccccc4c3)nc(-c3ccc4ccc5ccccc5c4c3)n2)cc1 ; c1ccc(-c2ccc3c(c2)oc2cccc(-c4ccc(-c5nc(-c6ccccc6)nc(-c6ccc7c(ccc8ccccc87)c6)n5)c5ccccc45)c23)cc1]

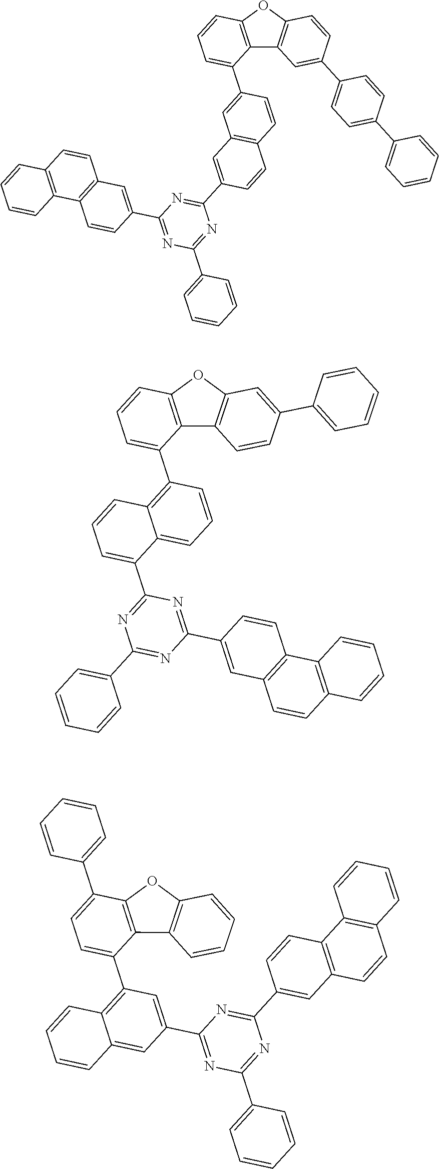 [SMILES: c1ccc(-c2ccc(-c3ccc4oc5cccc(-c6ccc7ccc(-c8nc(-c9ccccc9)nc(-c9ccc%10c(ccc%11ccccc%11%10)c9)n8)cc7c6)c5c4c3)cc2)cc1 ; c1ccc(-c2ccc3c(c2)oc2cccc(-c4cccc5c(-c6nc(-c7ccccc7)nc(-c7ccc8c(ccc9ccccc98)c7)n6)cccc45)c23)cc1 ; c1ccc(-c2nc(-c3cc(-c4ccc(-c5ccccc5)c5oc6ccccc6c45)c4ccccc4c3)nc(-c3ccc4c(ccc5ccccc54)c3)n2)cc1]

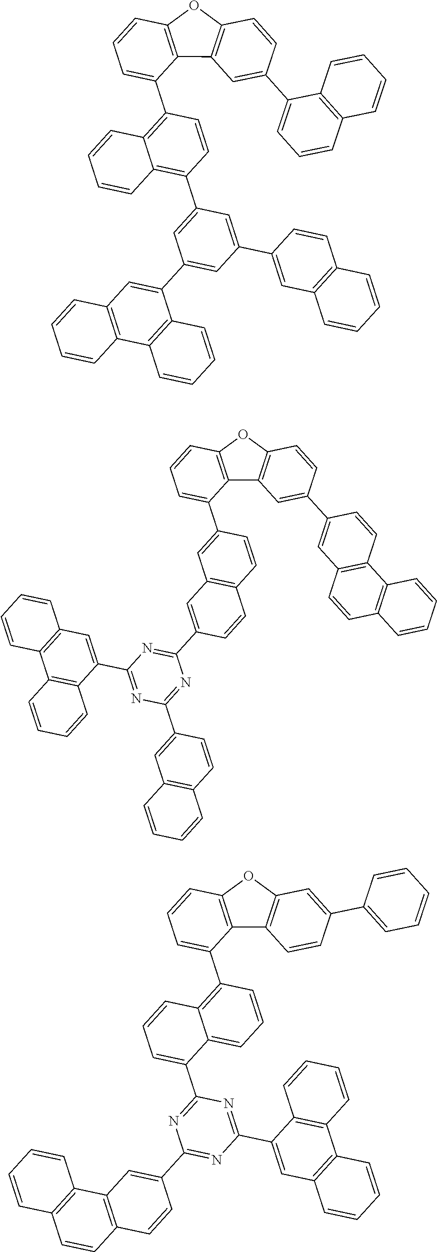 [SMILES: c1ccc2cc(-c3cc(-c4ccc(-c5cccc6oc7ccc(-c8cccc9ccccc89)cc7c56)c5ccccc45)cc(-c4cc5ccccc5c5ccccc45)c3)ccc2c1 ; c1ccc2cc(-c3nc(-c4ccc5ccc(-c6cccc7oc8ccc(-c9ccc%10c(ccc%11ccccc%11%10)c9)cc8c67)cc5c4)nc(-c4cc5ccccc5c5ccccc45)n3)ccc2c1 ; c1ccc(-c2ccc3c(c2)oc2cccc(-c4cccc5c(-c6nc(-c7ccc8ccc9ccccc9c8c7)nc(-c7cc8ccccc8c8ccccc78)n6)cccc45)c23)cc1]

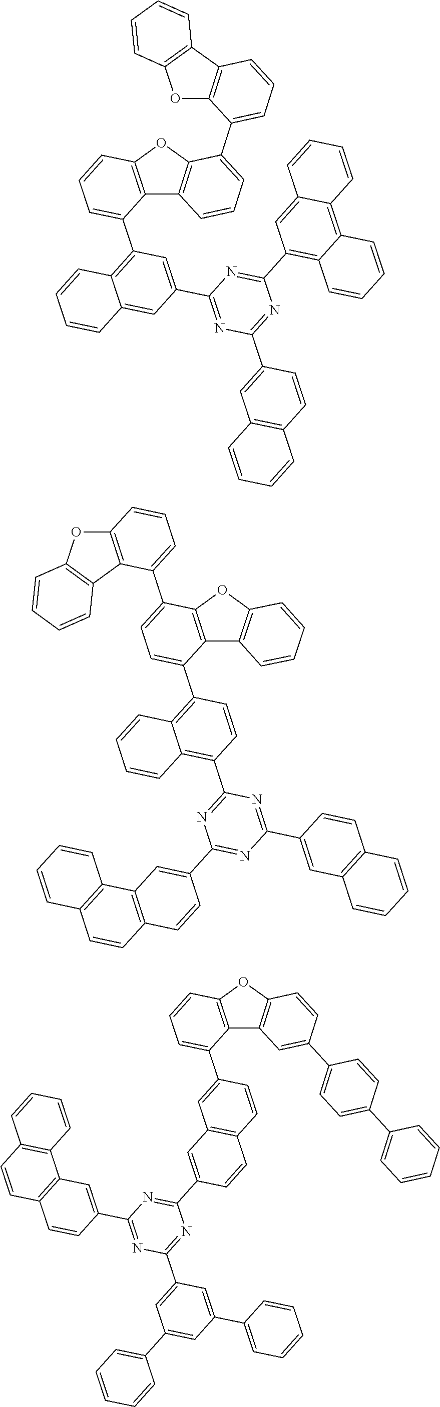 [SMILES: c1ccc2cc(-c3nc(-c4cc(-c5cccc6oc7c(-c8cccc9c8oc8ccccc89)cccc7c56)c5ccccc5c4)nc(-c4cc5ccccc5c5ccccc45)n3)ccc2c1 ; c1ccc2cc(-c3nc(-c4ccc5ccc6ccccc6c5c4)nc(-c4ccc(-c5ccc(-c6cccc7oc8ccccc8c67)c6oc7ccccc7c56)c5ccccc45)n3)ccc2c1 ; c1ccc(-c2ccc(-c3ccc4oc5cccc(-c6ccc7ccc(-c8nc(-c9cc(-c%10ccccc%10)cc(-c%10ccccc%10)c9)nc(-c9ccc%10ccc%11ccccc%11c%10c9)n8)cc7c6)c5c4c3)cc2)cc1]

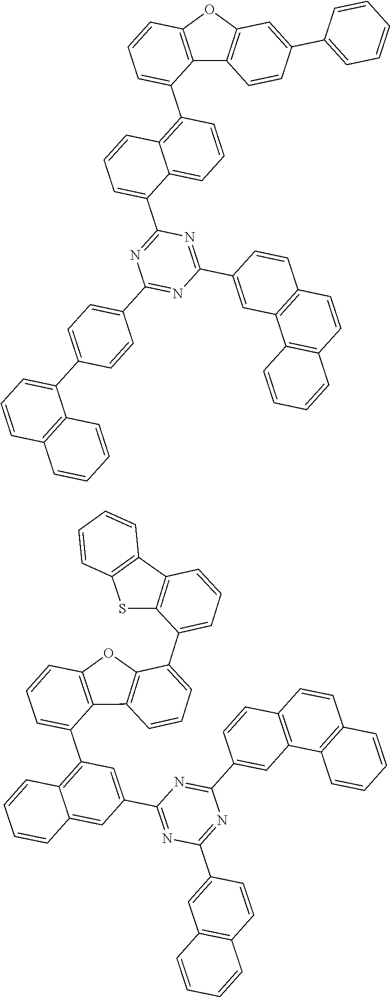

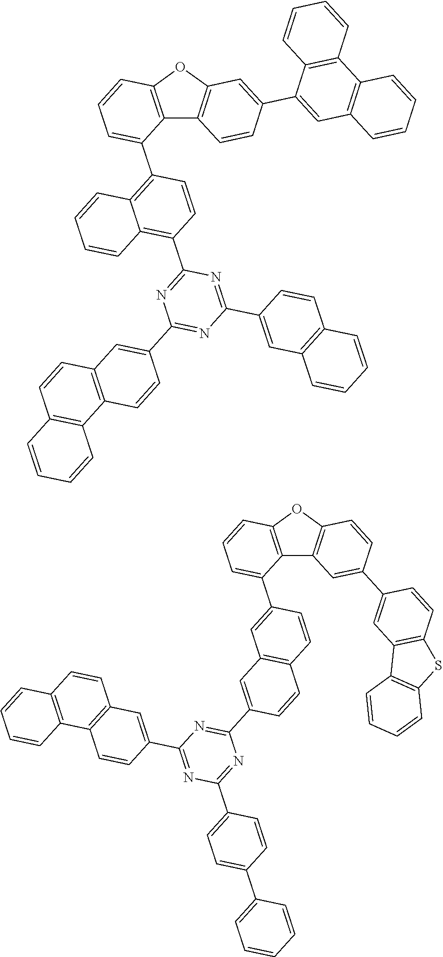 [SMILES: c1ccc2cc(-c3nc(-c4ccc5c(ccc6ccccc65)c4)nc(-c4ccc(-c5cccc6oc7cc(-c8cc9ccccc9c9ccccc89)ccc7c56)c5ccccc45)n3)ccc2c1 ; c1ccc(-c2ccc(-c3nc(-c4ccc5ccc(-c6cccc7oc8ccc(-c9ccc%10sc%11ccccc%11c%10c9)cc8c67)cc5c4)nc(-c4ccc5c(ccc6ccccc65)c4)n3)cc2)cc1]

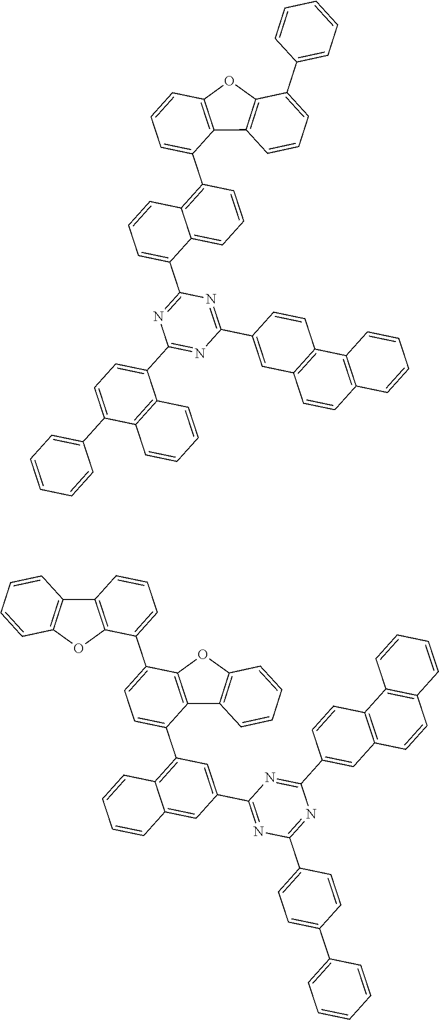 [SMILES: c1ccc(-c2ccc(-c3nc(-c4ccc5c(ccc6ccccc65)c4)nc(-c4cccc5c(-c6cccc7oc8c(-c9ccccc9)cccc8c67)cccc45)n3)c3ccccc23)cc1 ; c1ccc(-c2ccc(-c3nc(-c4cc(-c5ccc(-c6cccc7c6oc6ccccc67)c6oc7ccccc7c56)c5ccccc5c4)nc(-c4ccc5c(ccc6ccccc65)c4)n3)cc2)cc1]

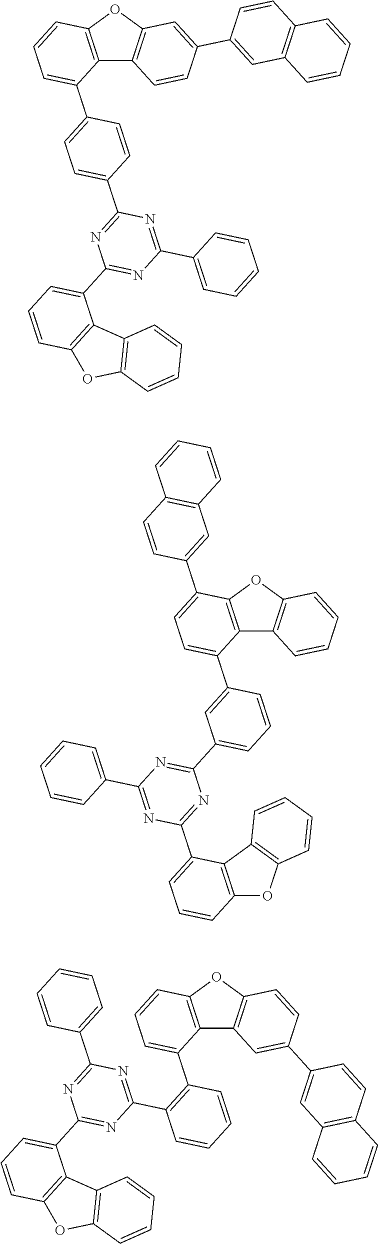 [SMILES: c1ccc(-c2nc(-c3ccc(-c4cccc5oc6cc(-c7ccc8ccccc8c7)ccc6c45)cc3)nc(-c3cccc4oc5ccccc5c34)n2)cc1 ; c1ccc(-c2nc(-c3cccc(-c4ccc(-c5ccc6ccccc6c5)c5oc6ccccc6c45)c3)nc(-c3cccc4oc5ccccc5c34)n2)cc1 ; c1ccc(-c2nc(-c3ccccc3-c3cccc4oc5ccc(-c6ccc7ccccc7c6)cc5c34)nc(-c3cccc4oc5ccccc5c34)n2)cc1]

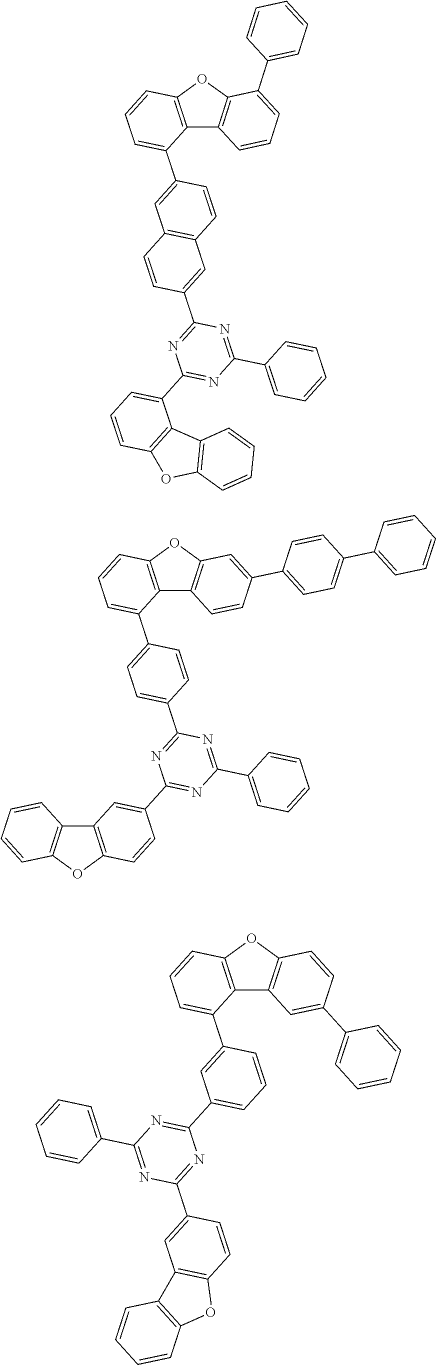 [SMILES: c1ccc(-c2nc(-c3ccc4cc(-c5cccc6oc7c(-c8ccccc8)cccc7c56)ccc4c3)nc(-c3cccc4oc5ccccc5c34)n2)cc1 ; c1ccc(-c2ccc(-c3ccc4c(c3)oc3cccc(-c5ccc(-c6nc(-c7ccccc7)nc(-c7ccc8oc9ccccc9c8c7)n6)cc5)c34)cc2)cc1 ; c1ccc(-c2ccc3oc4cccc(-c5cccc(-c6nc(-c7ccccc7)nc(-c7ccc8oc9ccccc9c8c7)n6)c5)c4c3c2)cc1]

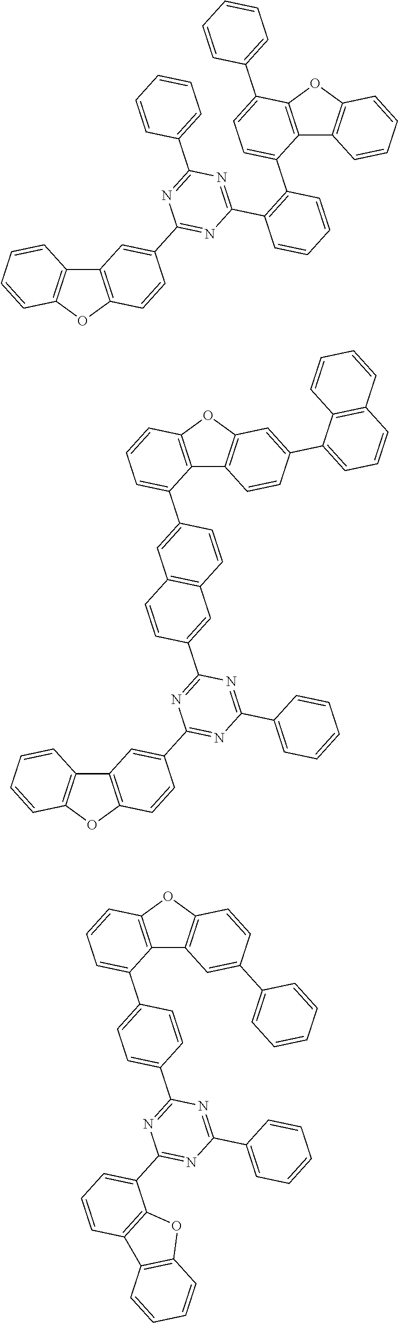 [SMILES: c1ccc(-c2nc(-c3ccc4oc5ccccc5c4c3)nc(-c3ccccc3-c3ccc(-c4ccccc4)c4oc5ccccc5c34)n2)cc1 ; c1ccc(-c2nc(-c3ccc4cc(-c5cccc6oc7cc(-c8cccc9ccccc89)ccc7c56)ccc4c3)nc(-c3ccc4oc5ccccc5c4c3)n2)cc1 ; c1ccc(-c2ccc3oc4cccc(-c5ccc(-c6nc(-c7ccccc7)nc(-c7cccc8c7oc7ccccc78)n6)cc5)c4c3c2)cc1]

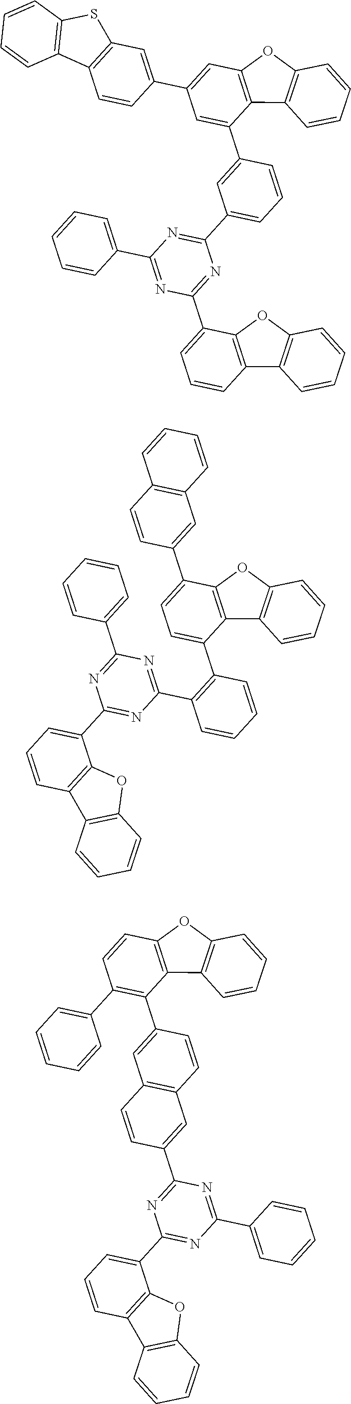 [SMILES: c1ccc(-c2nc(-c3cccc(-c4cc(-c5ccc6c(c5)sc5ccccc56)cc5oc6ccccc6c45)c3)nc(-c3cccc4c3oc3ccccc34)n2)cc1 ; c1ccc(-c2nc(-c3ccccc3-c3ccc(-c4ccc5ccccc5c4)c4oc5ccccc5c34)nc(-c3cccc4c3oc3ccccc34)n2)cc1 ; c1ccc(-c2nc(-c3ccc4cc(-c5c(-c6ccccc6)ccc6oc7ccccc7c56)ccc4c3)nc(-c3cccc4c3oc3ccccc34)n2)cc1]

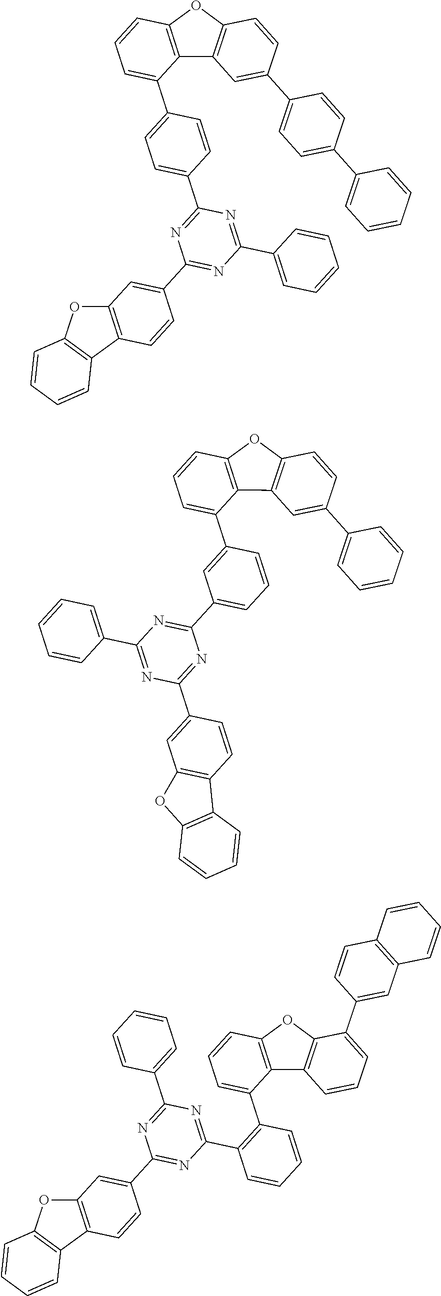 [SMILES: c1ccc(-c2ccc(-c3ccc4oc5cccc(-c6ccc(-c7nc(-c8ccccc8)nc(-c8ccc9c(c8)oc8ccccc89)n7)cc6)c5c4c3)cc2)cc1 ; c1ccc(-c2ccc3oc4cccc(-c5cccc(-c6nc(-c7ccccc7)nc(-c7ccc8c(c7)oc7ccccc78)n6)c5)c4c3c2)cc1 ; c1ccc(-c2nc(-c3ccc4c(c3)oc3ccccc34)nc(-c3ccccc3-c3cccc4oc5c(-c6ccc7ccccc7c6)cccc5c34)n2)cc1]

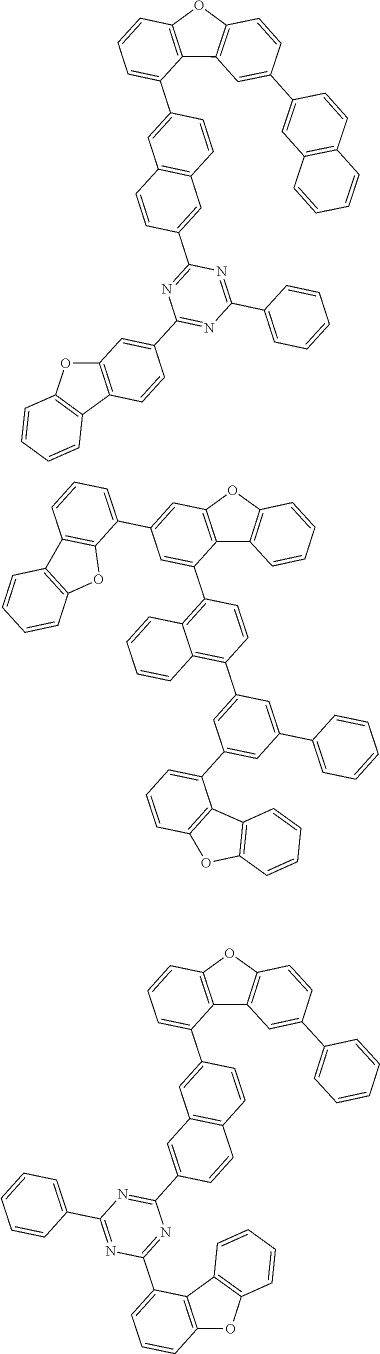

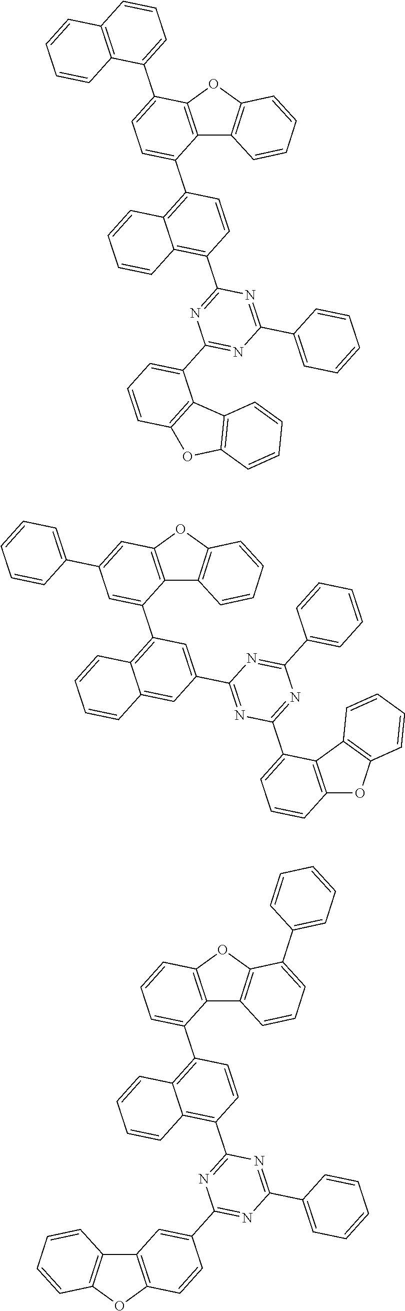 [SMILES: c1ccc(-c2nc(-c3ccc(-c4ccc(-c5cccc6ccccc56)c5oc6ccccc6c45)c4ccccc34)nc(-c3cccc4oc5ccccc5c34)n2)cc1 ; c1ccc(-c2cc(-c3cc(-c4nc(-c5ccccc5)nc(-c5cccc6oc7ccccc7c56)n4)cc4ccccc34)c3c(c2)oc2ccccc23)cc1 ; c1ccc(-c2nc(-c3ccc4oc5ccccc5c4c3)nc(-c3ccc(-c4cccc5oc6c(-c7ccccc7)cccc6c45)c4ccccc34)n2)cc1]

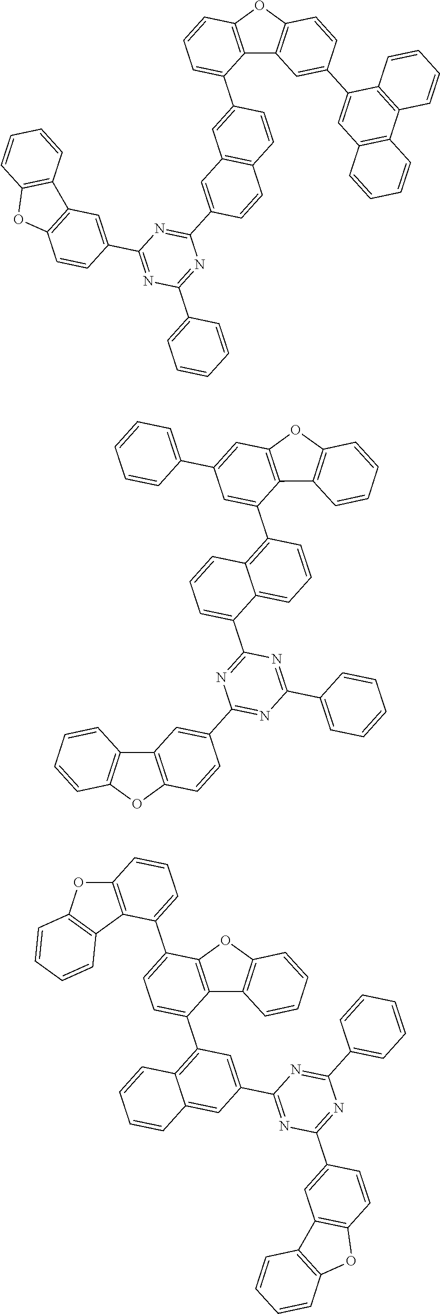 [SMILES: c1ccc(-c2nc(-c3ccc4ccc(-c5cccc6oc7ccc(-c8cc9ccccc9c9ccccc89)cc7c56)cc4c3)nc(-c3ccc4oc5ccccc5c4c3)n2)cc1 ; c1ccc(-c2cc(-c3cccc4c(-c5nc(-c6ccccc6)nc(-c6ccc7oc8ccccc8c7c6)n5)cccc34)c3c(c2)oc2ccccc23)cc1 ; c1ccc(-c2nc(-c3cc(-c4ccc(-c5cccc6oc7ccccc7c56)c5oc6ccccc6c45)c4ccccc4c3)nc(-c3ccc4oc5ccccc5c4c3)n2)cc1]

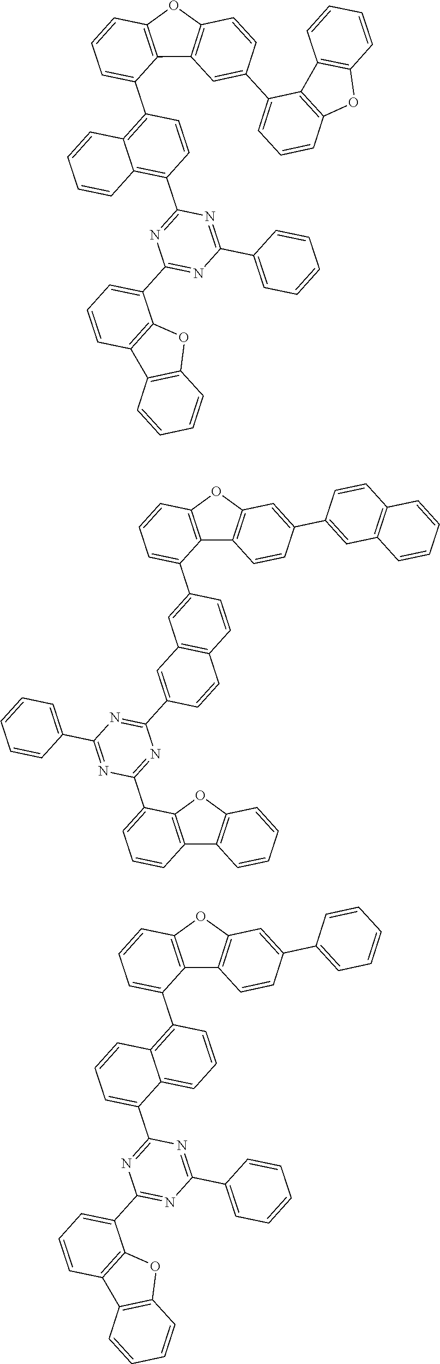 [SMILES: c1ccc(-c2nc(-c3ccc(-c4cccc5oc6ccc(-c7cccc8oc9ccccc9c78)cc6c45)c4ccccc34)nc(-c3cccc4c3oc3ccccc34)n2)cc1 ; c1ccc(-c2nc(-c3ccc4ccc(-c5cccc6oc7cc(-c8ccc9ccccc9c8)ccc7c56)cc4c3)nc(-c3cccc4c3oc3ccccc34)n2)cc1 ; c1ccc(-c2ccc3c(c2)oc2cccc(-c4cccc5c(-c6nc(-c7ccccc7)nc(-c7cccc8c7oc7ccccc78)n6)cccc45)c23)cc1]

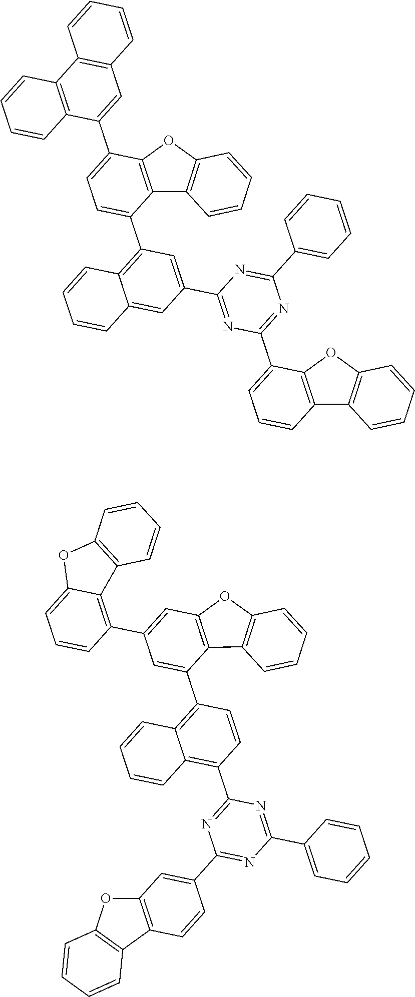 [SMILES: c1ccc(-c2nc(-c3cc(-c4ccc(-c5cc6ccccc6c6ccccc56)c5oc6ccccc6c45)c4ccccc4c3)nc(-c3cccc4c3oc3ccccc34)n2)cc1 ; c1ccc(-c2nc(-c3ccc4c(c3)oc3ccccc34)nc(-c3ccc(-c4cc(-c5cccc6oc7ccccc7c56)cc5oc6ccccc6c45)c4ccccc34)n2)cc1]

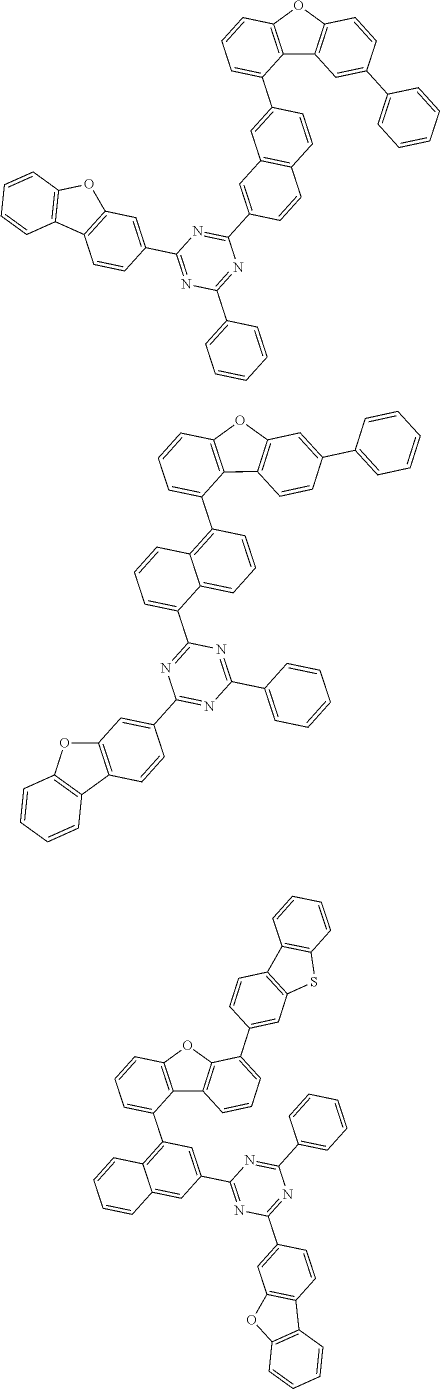

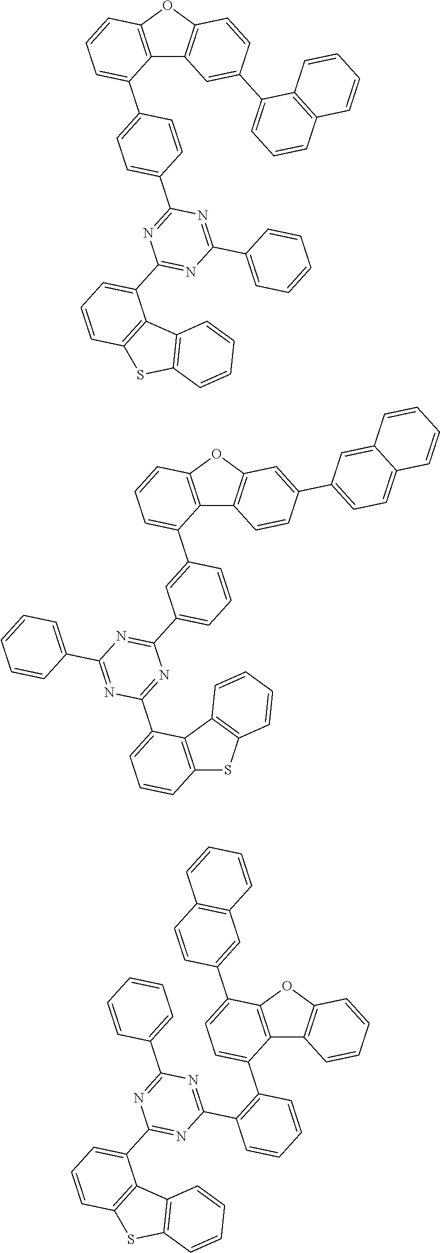 [SMILES: c1ccc(-c2nc(-c3ccc(-c4cccc5oc6ccc(-c7cccc8ccccc78)cc6c45)cc3)nc(-c3cccc4sc5ccccc5c34)n2)cc1 ; c1ccc(-c2nc(-c3cccc(-c4cccc5oc6cc(-c7ccc8ccccc8c7)ccc6c45)c3)nc(-c3cccc4sc5ccccc5c34)n2)cc1 ; c1ccc(-c2nc(-c3ccccc3-c3ccc(-c4ccc5ccccc5c4)c4oc5ccccc5c34)nc(-c3cccc4sc5ccccc5c34)n2)cc1]

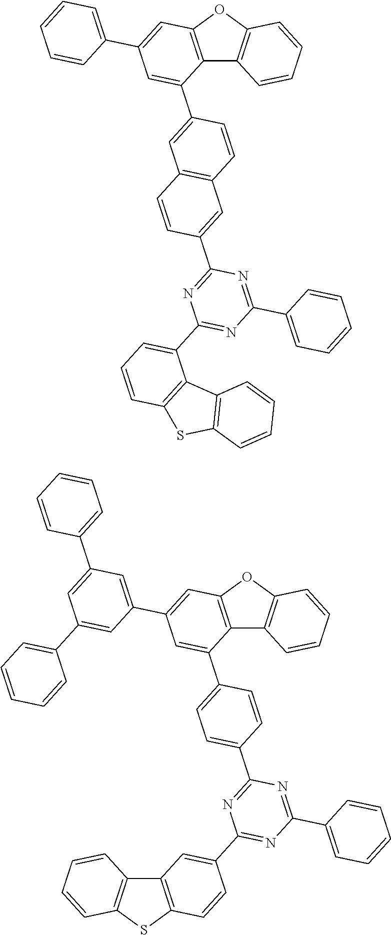 [SMILES: c1ccc(-c2cc(-c3ccc4cc(-c5nc(-c6ccccc6)nc(-c6cccc7sc8ccccc8c67)n5)ccc4c3)c3c(c2)oc2ccccc23)cc1 ; c1ccc(-c2cc(-c3ccccc3)cc(-c3cc(-c4ccc(-c5nc(-c6ccccc6)nc(-c6ccc7sc8ccccc8c7c6)n5)cc4)c4c(c3)oc3ccccc34)c2)cc1]

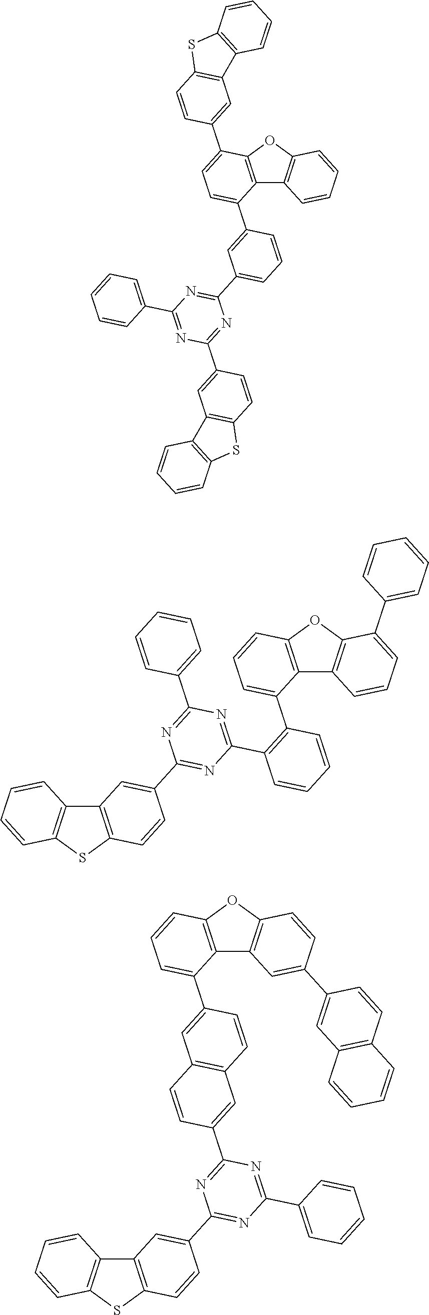 [SMILES: c1ccc(-c2nc(-c3cccc(-c4ccc(-c5ccc6sc7ccccc7c6c5)c5oc6ccccc6c45)c3)nc(-c3ccc4sc5ccccc5c4c3)n2)cc1 ; c1ccc(-c2nc(-c3ccc4sc5ccccc5c4c3)nc(-c3ccccc3-c3cccc4oc5c(-c6ccccc6)cccc5c34)n2)cc1 ; c1ccc(-c2nc(-c3ccc4cc(-c5cccc6oc7ccc(-c8ccc9ccccc9c8)cc7c56)ccc4c3)nc(-c3ccc4sc5ccccc5c4c3)n2)cc1]

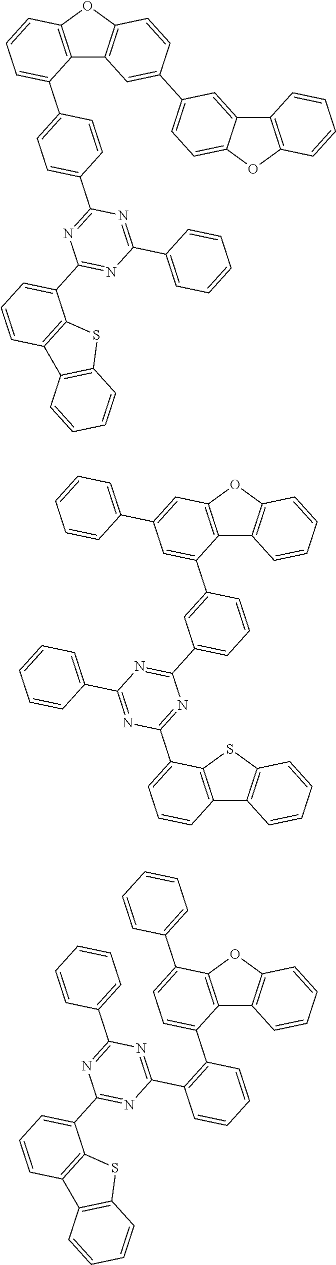

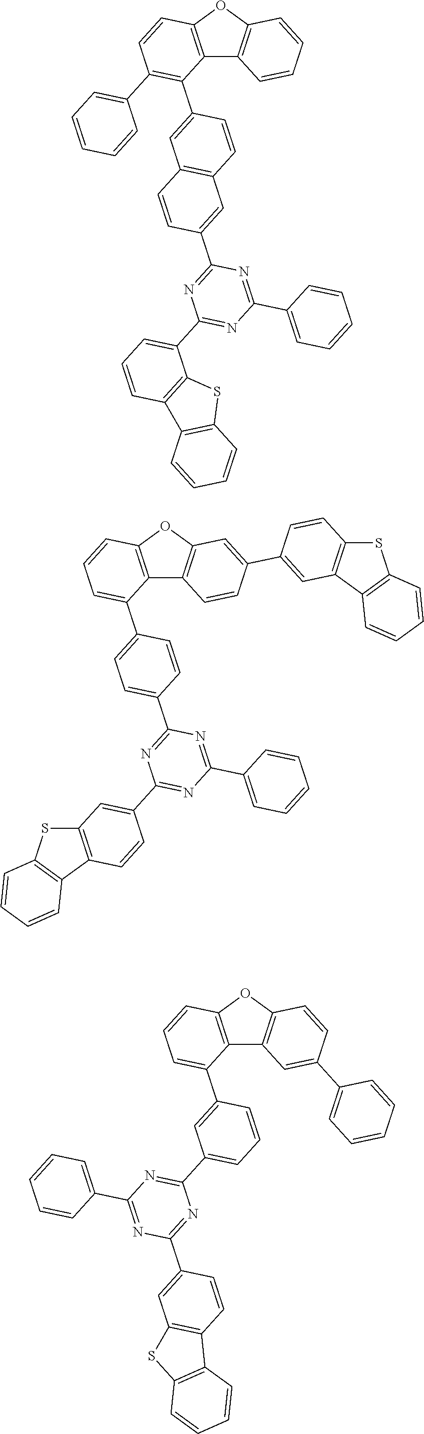 [SMILES: c1ccc(-c2nc(-c3ccc4cc(-c5c(-c6ccccc6)ccc6oc7ccccc7c56)ccc4c3)nc(-c3cccc4c3sc3ccccc34)n2)cc1 ; c1ccc(-c2nc(-c3ccc(-c4cccc5oc6cc(-c7ccc8sc9ccccc9c8c7)ccc6c45)cc3)nc(-c3ccc4c(c3)sc3ccccc34)n2)cc1 ; c1ccc(-c2ccc3oc4cccc(-c5cccc(-c6nc(-c7ccccc7)nc(-c7ccc8c(c7)sc7ccccc78)n6)c5)c4c3c2)cc1]

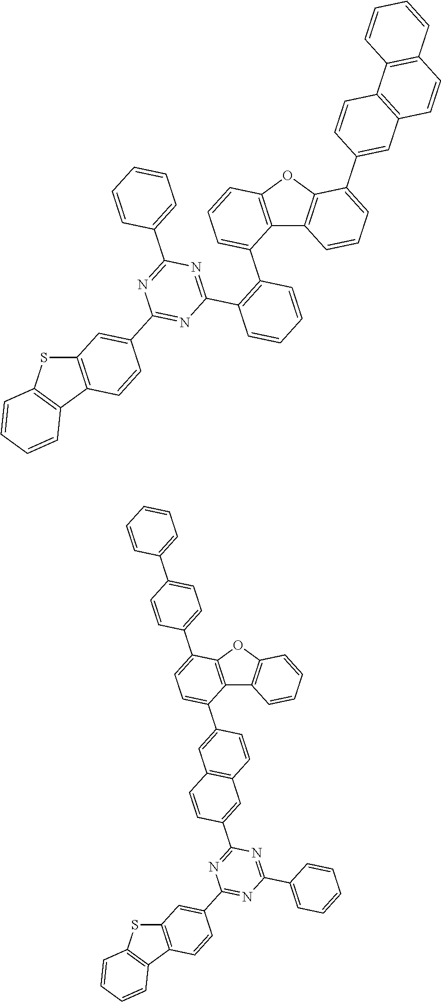 [SMILES: c1ccc(-c2nc(-c3ccc4c(c3)sc3ccccc34)nc(-c3ccccc3-c3cccc4oc5c(-c6ccc7c(ccc8ccccc87)c6)cccc5c34)n2)cc1 ; c1ccc(-c2ccc(-c3ccc(-c4ccc5cc(-c6nc(-c7ccccc7)nc(-c7ccc8c(c7)sc7ccccc78)n6)ccc5c4)c4c3oc3ccccc34)cc2)cc1]

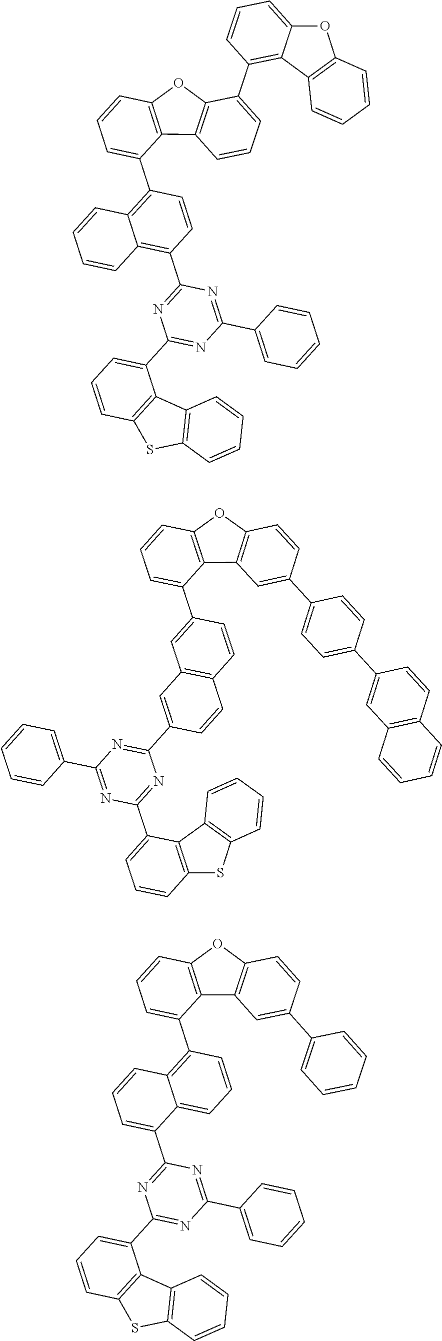 [SMILES: c1ccc(-c2nc(-c3ccc(-c4cccc5oc6c(-c7cccc8oc9ccccc9c78)cccc6c45)c4ccccc34)nc(-c3cccc4sc5ccccc5c34)n2)cc1 ; c1ccc(-c2nc(-c3ccc4ccc(-c5cccc6oc7ccc(-c8ccc(-c9ccc%10ccccc%10c9)cc8)cc7c56)cc4c3)nc(-c3cccc4sc5ccccc5c34)n2)cc1 ; c1ccc(-c2ccc3oc4cccc(-c5cccc6c(-c7nc(-c8ccccc8)nc(-c8cccc9sc%10ccccc%10c89)n7)cccc56)c4c3c2)cc1]

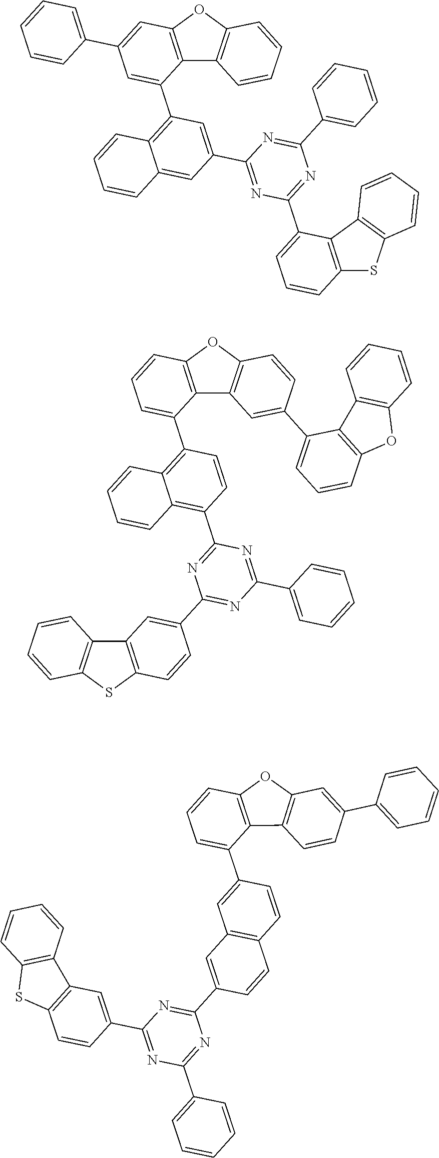 [SMILES: c1ccc(-c2cc(-c3cc(-c4nc(-c5ccccc5)nc(-c5cccc6sc7ccccc7c56)n4)cc4ccccc34)c3c(c2)oc2ccccc23)cc1 ; c1ccc(-c2nc(-c3ccc4sc5ccccc5c4c3)nc(-c3ccc(-c4cccc5oc6ccc(-c7cccc8oc9ccccc9c78)cc6c45)c4ccccc34)n2)cc1 ; c1ccc(-c2ccc3c(c2)oc2cccc(-c4ccc5ccc(-c6nc(-c7ccccc7)nc(-c7ccc8sc9ccccc9c8c7)n6)cc5c4)c23)cc1]

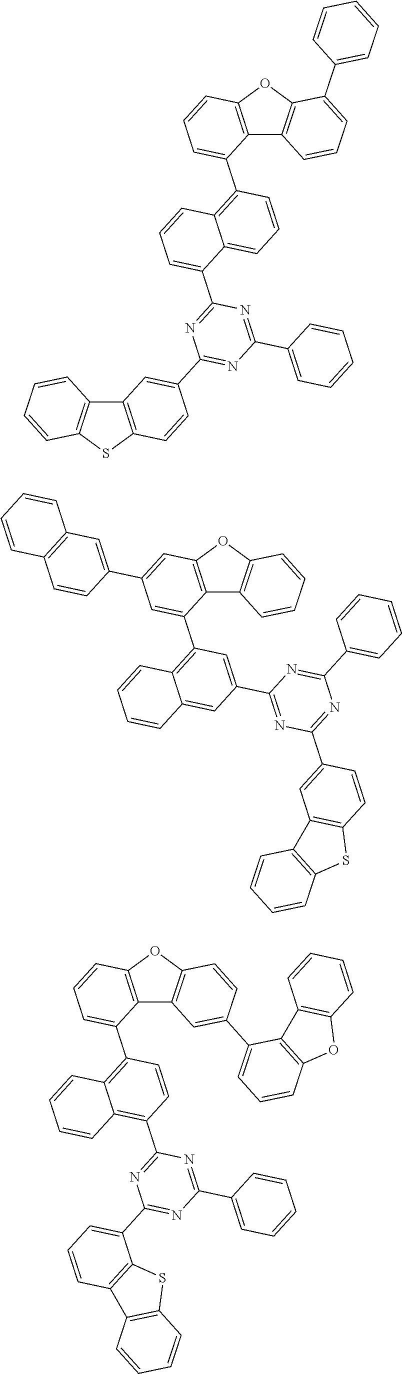 [SMILES: c1ccc(-c2nc(-c3ccc4sc5ccccc5c4c3)nc(-c3cccc4c(-c5cccc6oc7c(-c8ccccc8)cccc7c56)cccc34)n2)cc1 ; c1ccc(-c2nc(-c3cc(-c4cc(-c5ccc6ccccc6c5)cc5oc6ccccc6c45)c4ccccc4c3)nc(-c3ccc4sc5ccccc5c4c3)n2)cc1 ; c1ccc(-c2nc(-c3ccc(-c4cccc5oc6ccc(-c7cccc8oc9ccccc9c78)cc6c45)c4ccccc34)nc(-c3cccc4c3sc3ccccc34)n2)cc1]

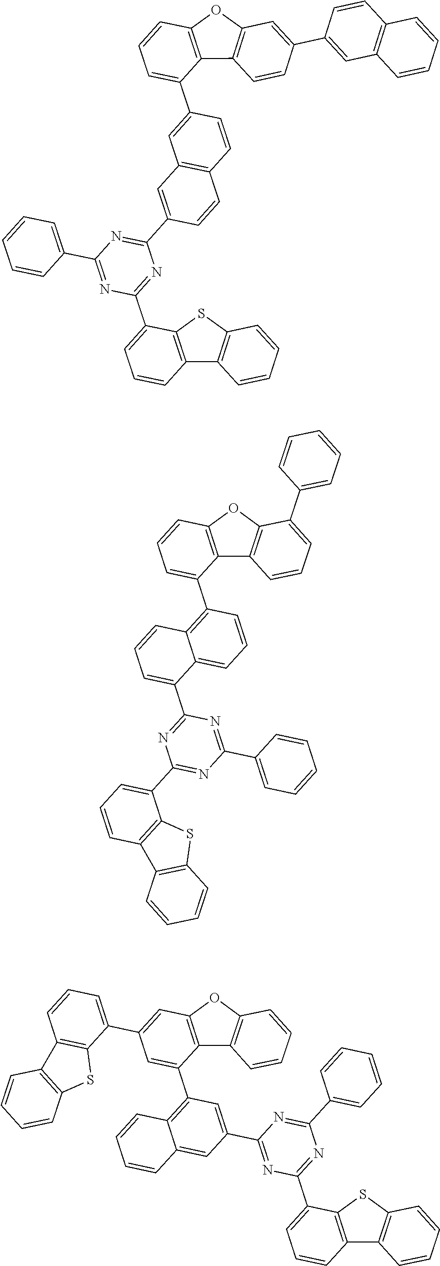 [SMILES: c1ccc(-c2nc(-c3ccc4ccc(-c5cccc6oc7cc(-c8ccc9ccccc9c8)ccc7c56)cc4c3)nc(-c3cccc4c3sc3ccccc34)n2)cc1 ; c1ccc(-c2nc(-c3cccc4c(-c5cccc6oc7c(-c8ccccc8)cccc7c56)cccc34)nc(-c3cccc4c3sc3ccccc34)n2)cc1 ; c1ccc(-c2nc(-c3cc(-c4cc(-c5cccc6c5sc5ccccc56)cc5oc6ccccc6c45)c4ccccc4c3)nc(-c3cccc4c3sc3ccccc34)n2)cc1]

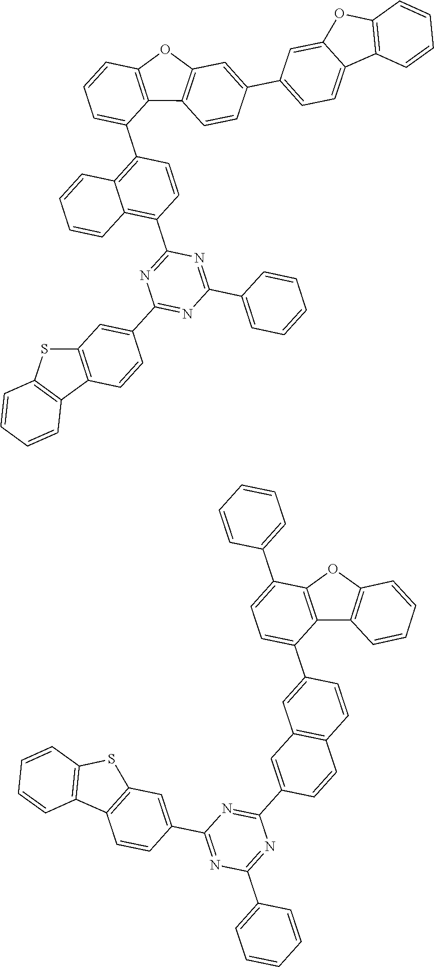 [SMILES: c1ccc(-c2nc(-c3ccc4c(c3)sc3ccccc34)nc(-c3ccc(-c4cccc5oc6cc(-c7ccc8c(c7)oc7ccccc78)ccc6c45)c4ccccc34)n2)cc1 ; c1ccc(-c2nc(-c3ccc4ccc(-c5ccc(-c6ccccc6)c6oc7ccccc7c56)cc4c3)nc(-c3ccc4c(c3)sc3ccccc34)n2)cc1]

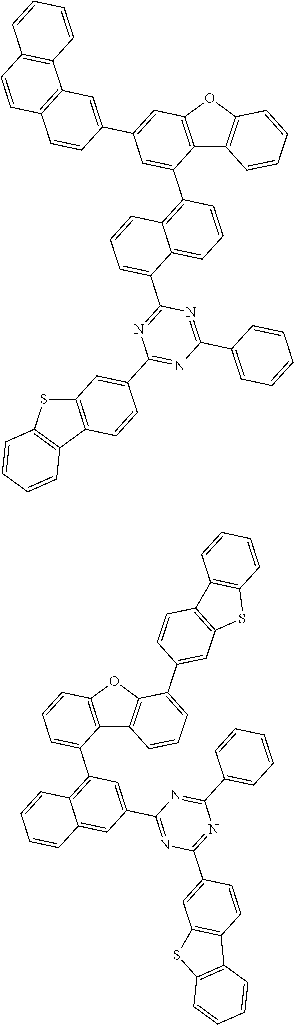 [SMILES: c1ccc(-c2nc(-c3ccc4c(c3)sc3ccccc34)nc(-c3cccc4c(-c5cc(-c6ccc7ccc8ccccc8c7c6)cc6oc7ccccc7c56)cccc34)n2)cc1 ; c1ccc(-c2nc(-c3cc(-c4cccc5oc6c(-c7ccc8c(c7)sc7ccccc78)cccc6c45)c4ccccc4c3)nc(-c3ccc4c(c3)sc3ccccc34)n2)cc1]

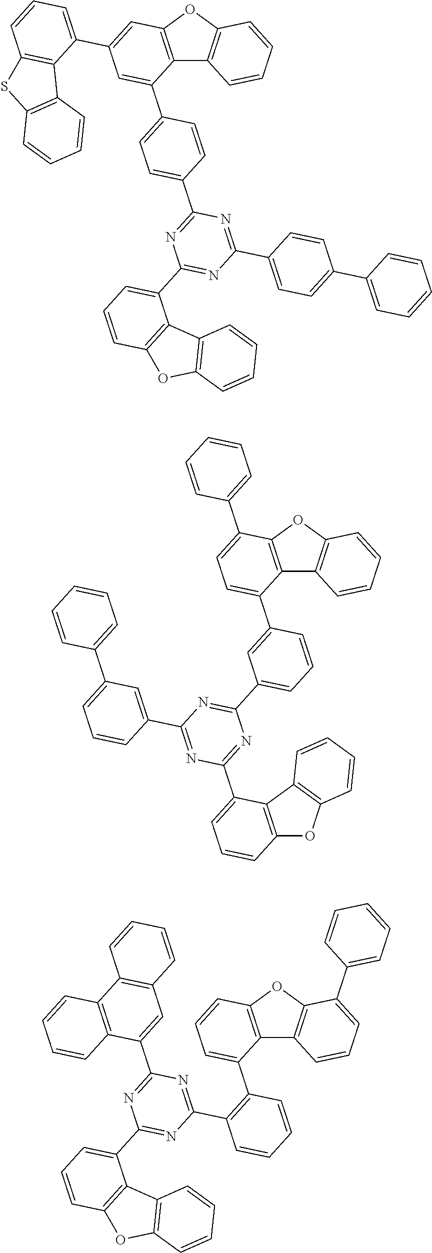 [SMILES: c1ccc(-c2ccc(-c3nc(-c4ccc(-c5cc(-c6cccc7sc8ccccc8c67)cc6oc7ccccc7c56)cc4)nc(-c4cccc5oc6ccccc6c45)n3)cc2)cc1 ; c1ccc(-c2cccc(-c3nc(-c4cccc(-c5ccc(-c6ccccc6)c6oc7ccccc7c56)c4)nc(-c4cccc5oc6ccccc6c45)n3)c2)cc1 ; c1ccc(-c2cccc3c2oc2cccc(-c4ccccc4-c4nc(-c5cc6ccccc6c6ccccc56)nc(-c5cccc6oc7ccccc7c56)n4)c23)cc1]

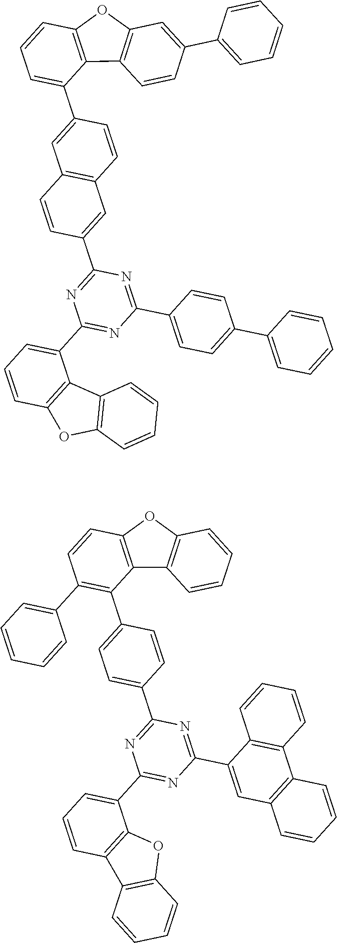 [SMILES: c1ccc(-c2ccc(-c3nc(-c4ccc5cc(-c6cccc7oc8cc(-c9ccccc9)ccc8c67)ccc5c4)nc(-c4cccc5oc6ccccc6c45)n3)cc2)cc1 ; c1ccc(-c2ccc3oc4ccccc4c3c2-c2ccc(-c3nc(-c4cc5ccccc5c5ccccc45)nc(-c4cccc5c4oc4ccccc45)n3)cc2)cc1]

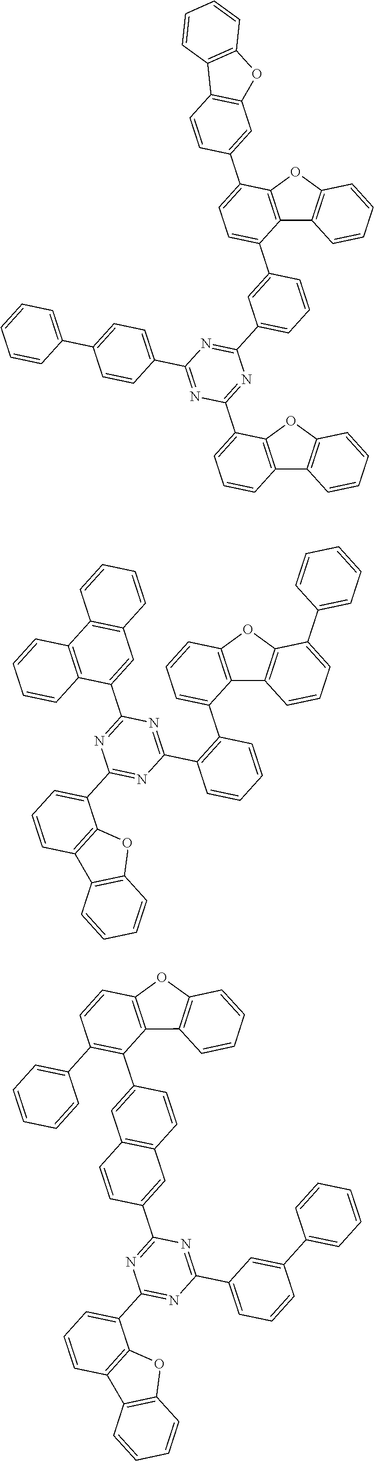 [SMILES: c1ccc(-c2ccc(-c3nc(-c4cccc(-c5ccc(-c6ccc7c(c6)oc6ccccc67)c6oc7ccccc7c56)c4)nc(-c4cccc5c4oc4ccccc45)n3)cc2)cc1 ; c1ccc(-c2cccc3c2oc2cccc(-c4ccccc4-c4nc(-c5cc6ccccc6c6ccccc56)nc(-c5cccc6c5oc5ccccc56)n4)c23)cc1 ; c1ccc(-c2cccc(-c3nc(-c4ccc5cc(-c6c(-c7ccccc7)ccc7oc8ccccc8c67)ccc5c4)nc(-c4cccc5c4oc4ccccc45)n3)c2)cc1]

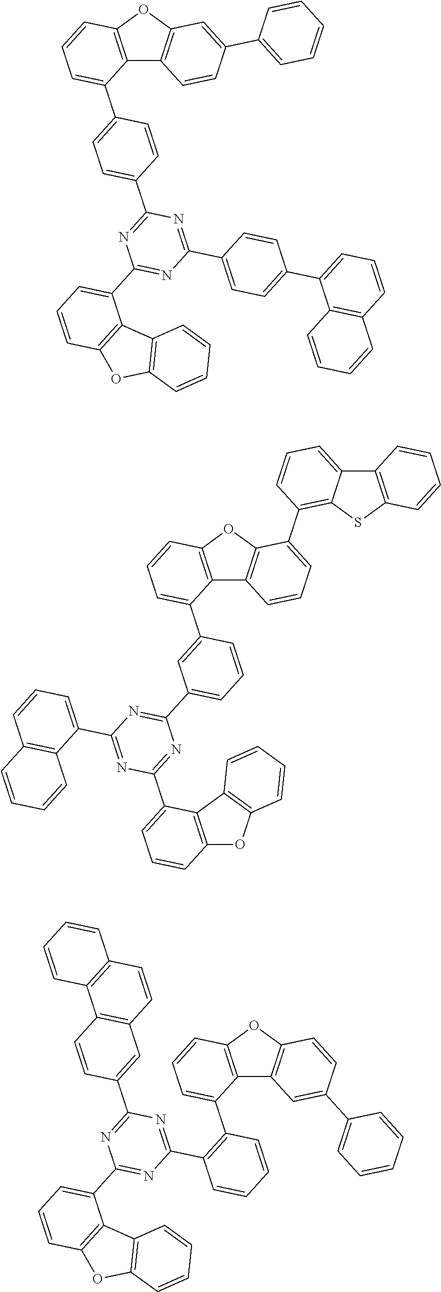 [SMILES: c1ccc(-c2ccc3c(c2)oc2cccc(-c4ccc(-c5nc(-c6ccc(-c7cccc8ccccc78)cc6)nc(-c6cccc7oc8ccccc8c67)n5)cc4)c23)cc1 ; c1cc(-c2nc(-c3cccc4ccccc34)nc(-c3cccc4oc5ccccc5c34)n2)cc(-c2cccc3oc4c(-c5cccc6c5sc5ccccc56)cccc4c23)c1 ; c1ccc(-c2ccc3oc4cccc(-c5ccccc5-c5nc(-c6ccc7c(ccc8ccccc87)c6)nc(-c6cccc7oc8ccccc8c67)n5)c4c3c2)cc1]

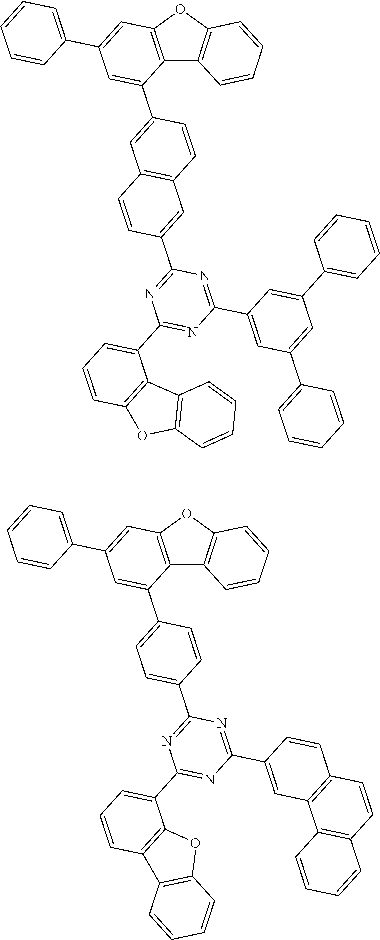 [SMILES: c1ccc(-c2cc(-c3ccccc3)cc(-c3nc(-c4ccc5cc(-c6cc(-c7ccccc7)cc7oc8ccccc8c67)ccc5c4)nc(-c4cccc5oc6ccccc6c45)n3)c2)cc1 ; c1ccc(-c2cc(-c3ccc(-c4nc(-c5ccc6ccc7ccccc7c6c5)nc(-c5cccc6c5oc5ccccc56)n4)cc3)c3c(c2)oc2ccccc23)cc1]

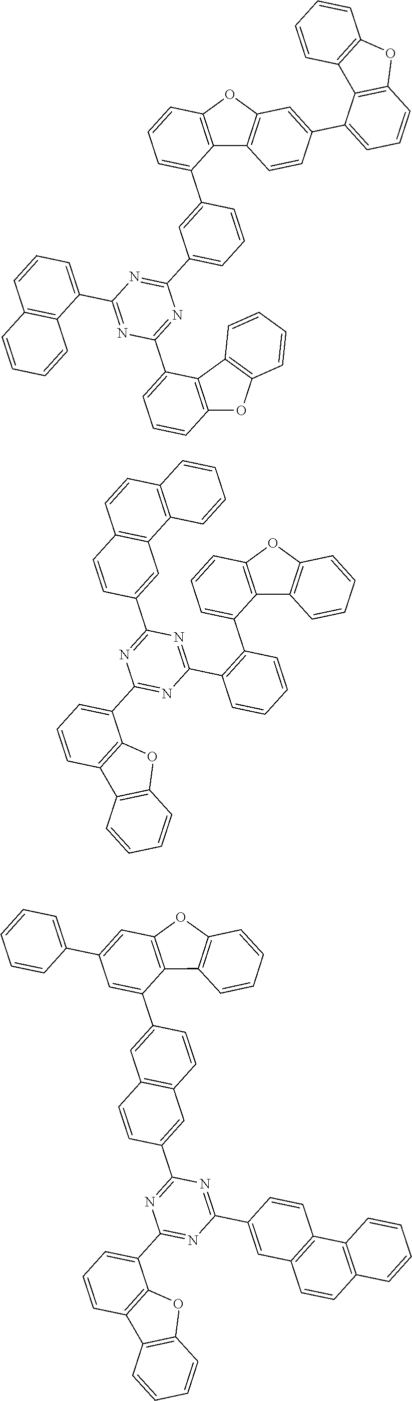

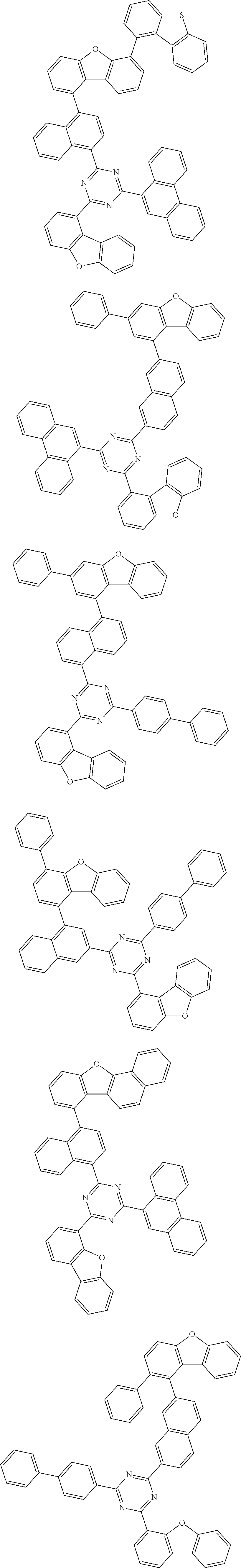

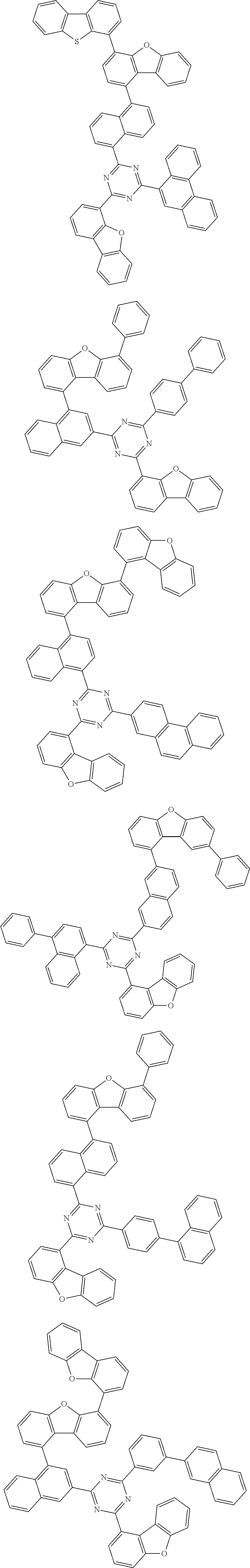

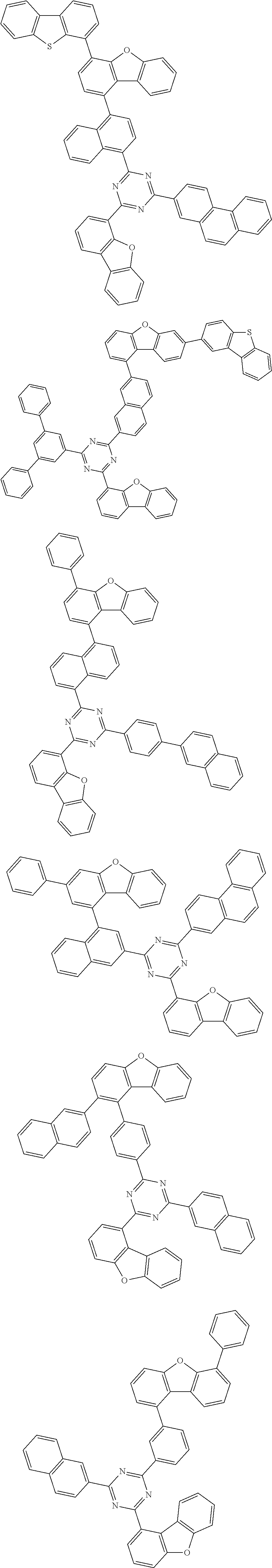

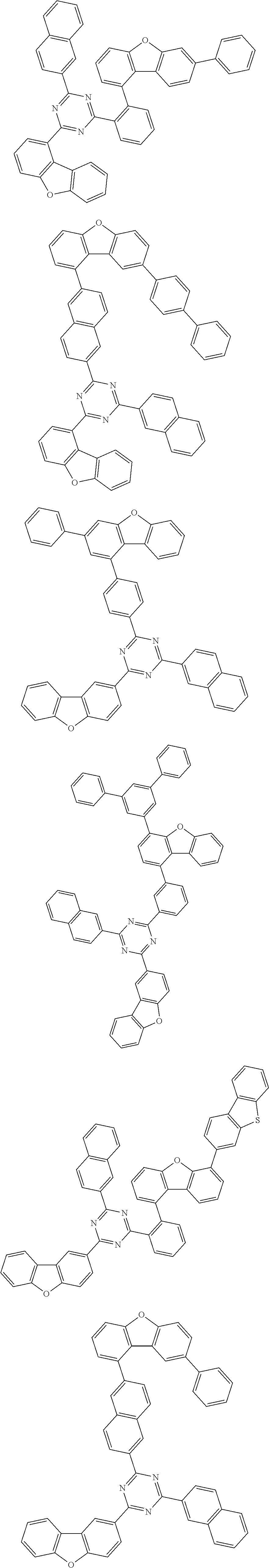

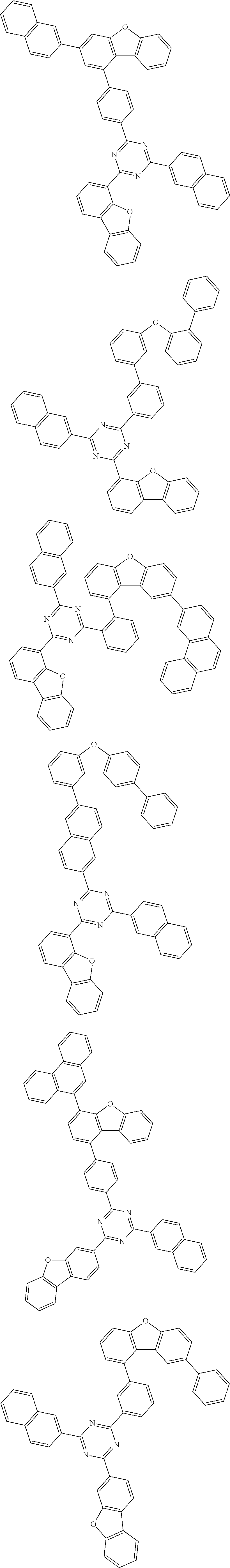

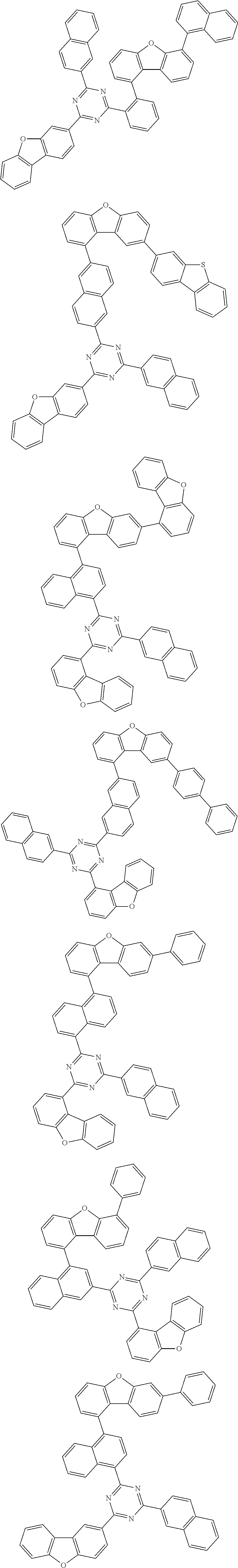

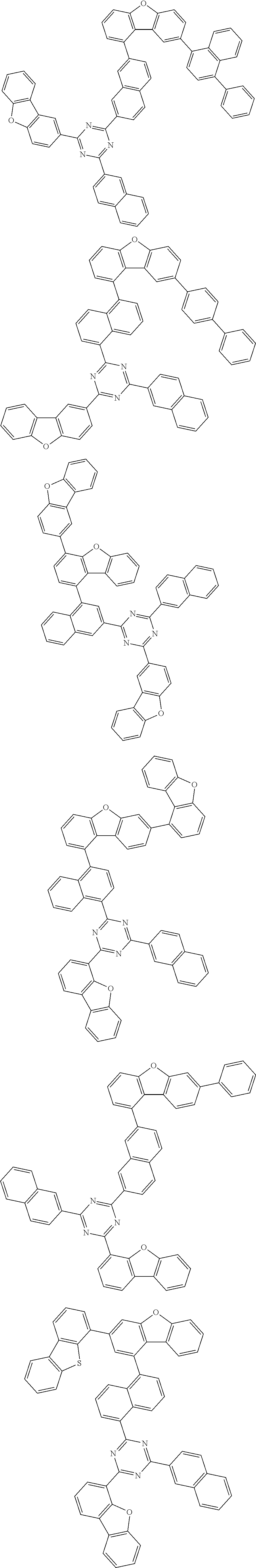

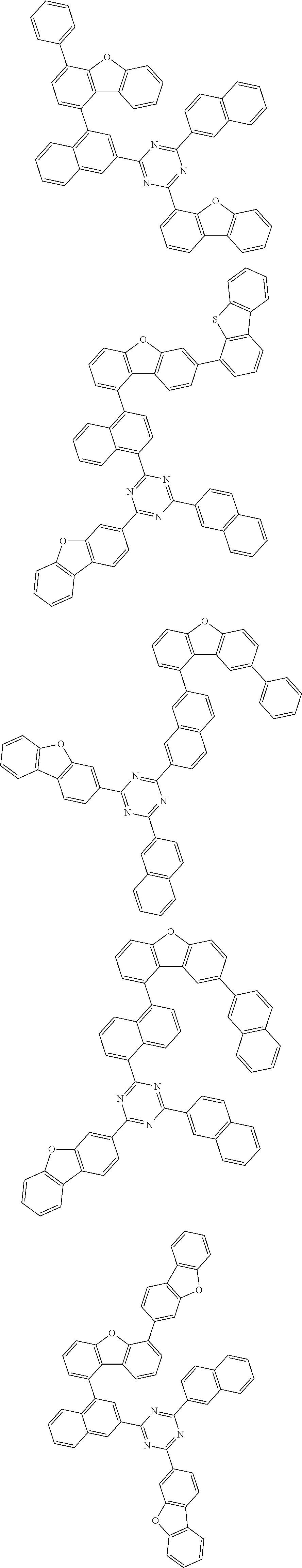

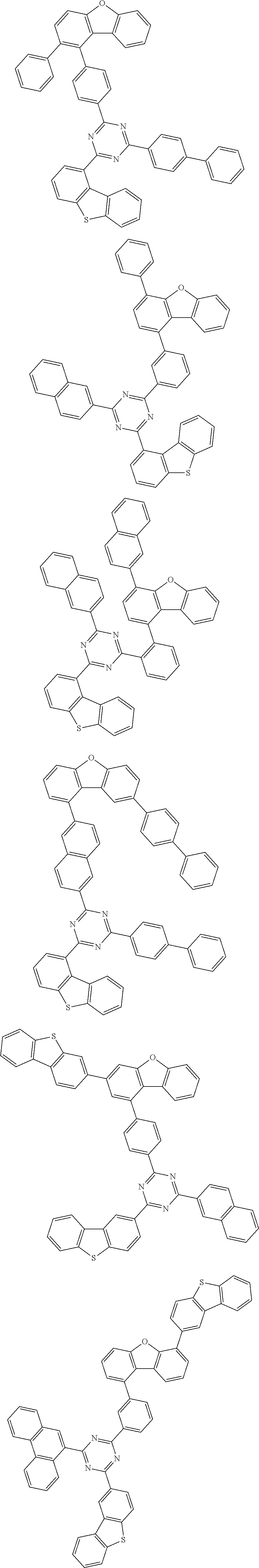

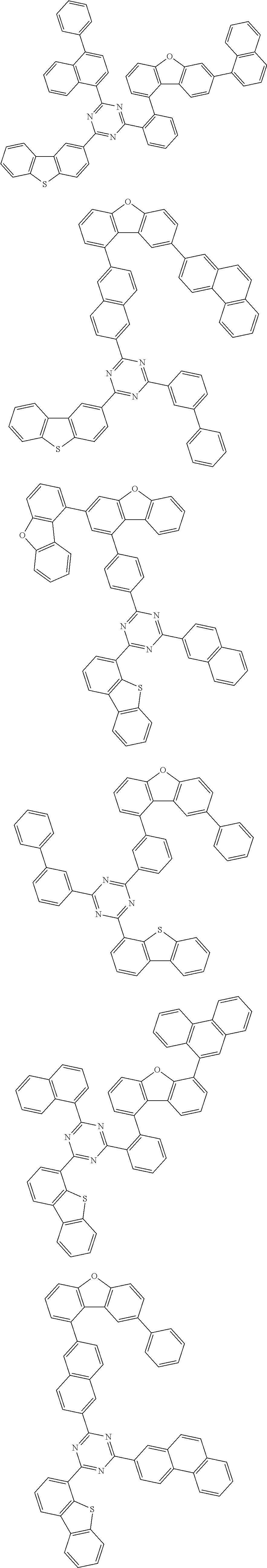

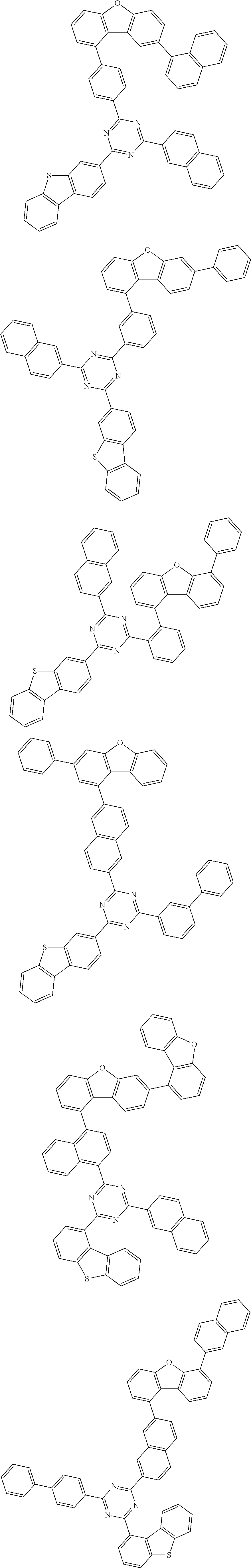

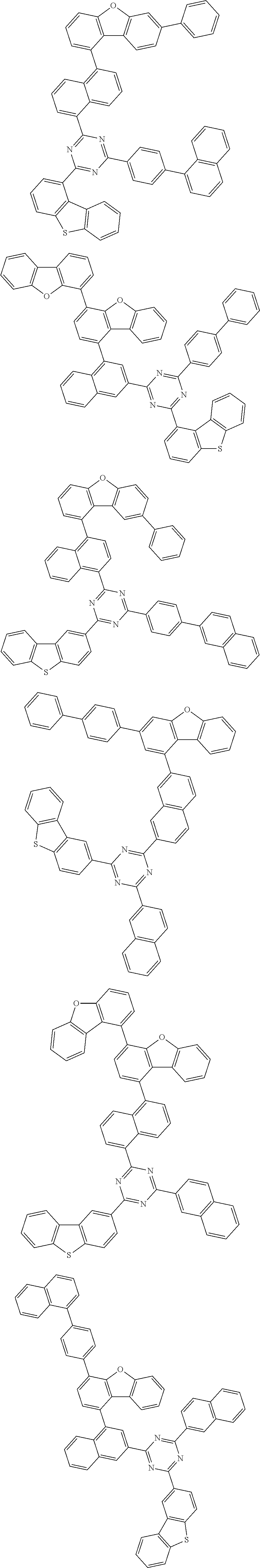

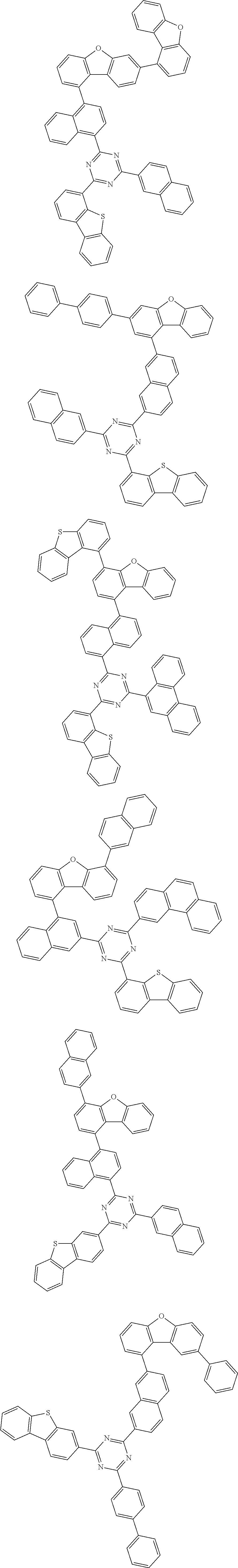

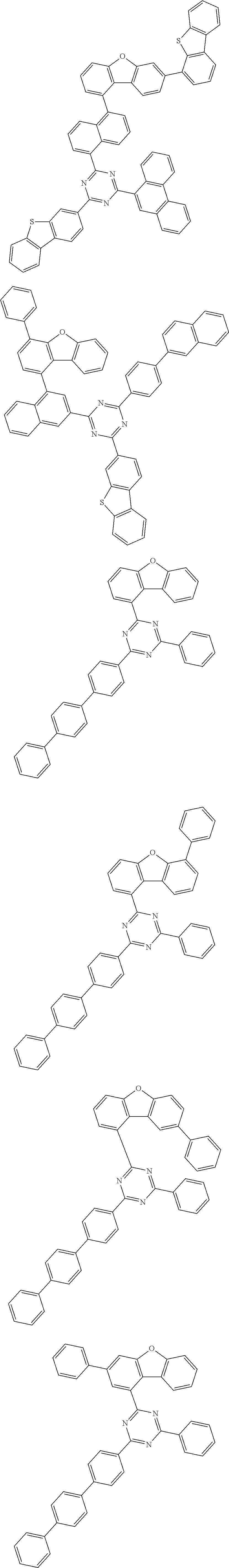

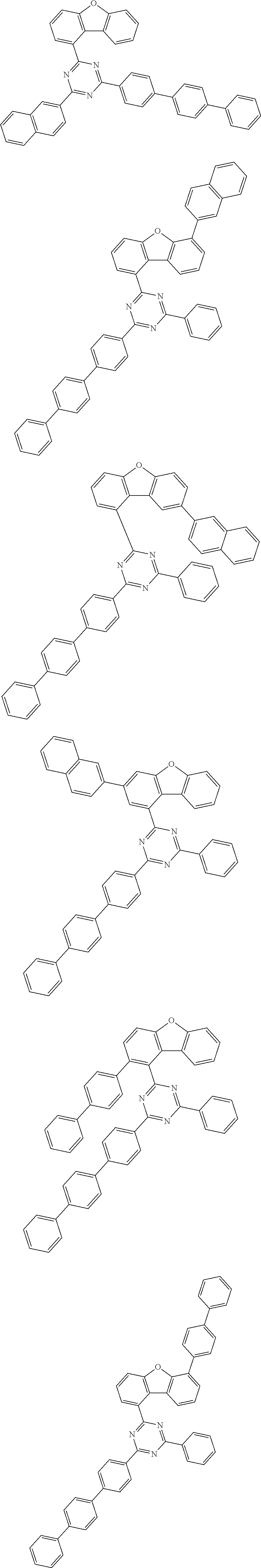

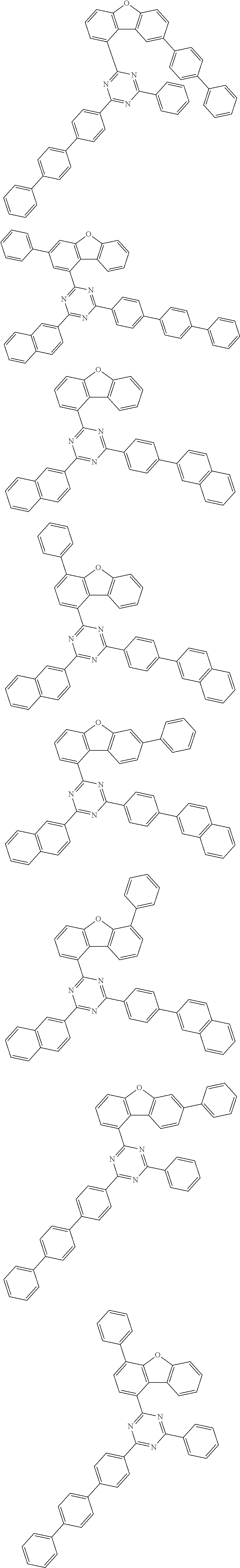

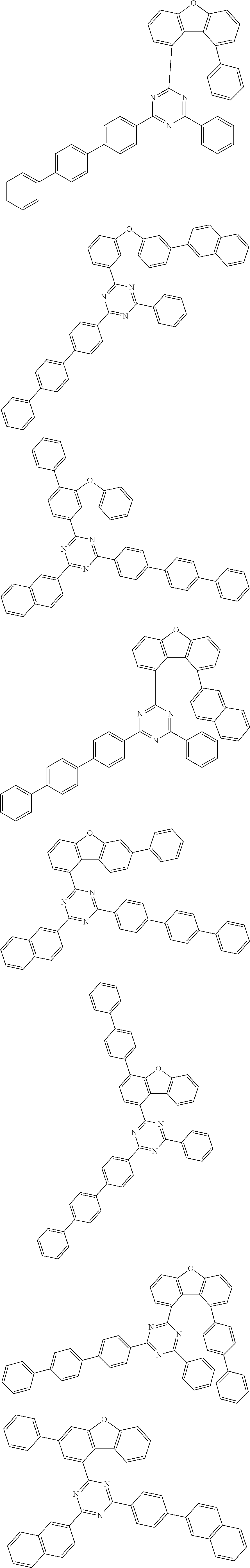

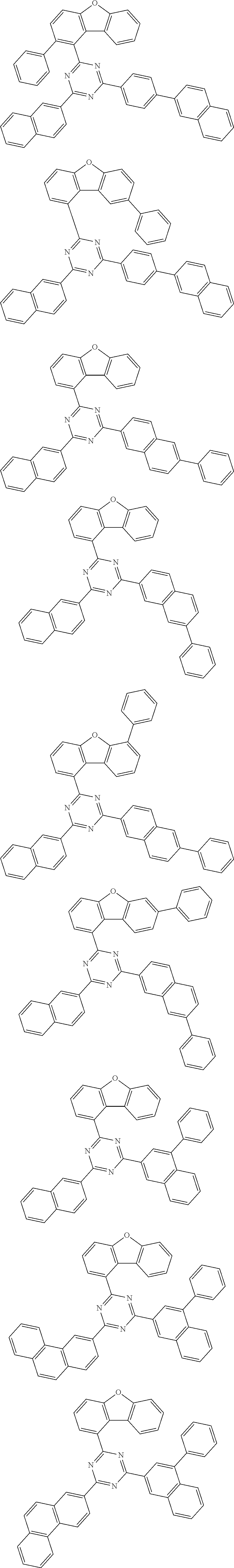

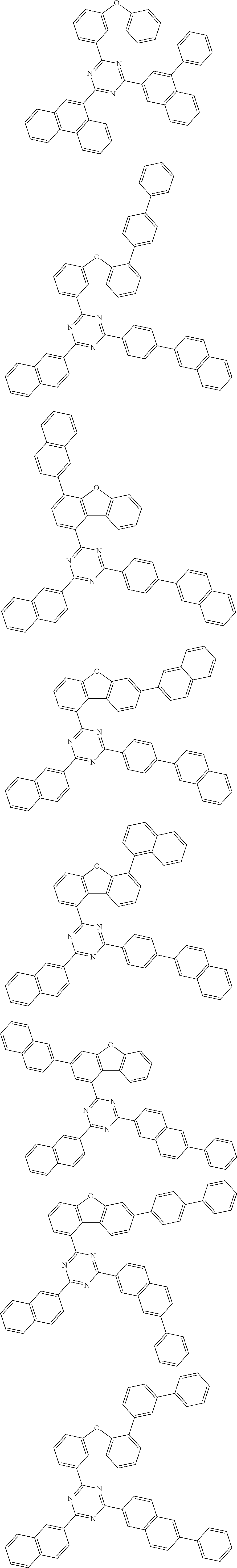

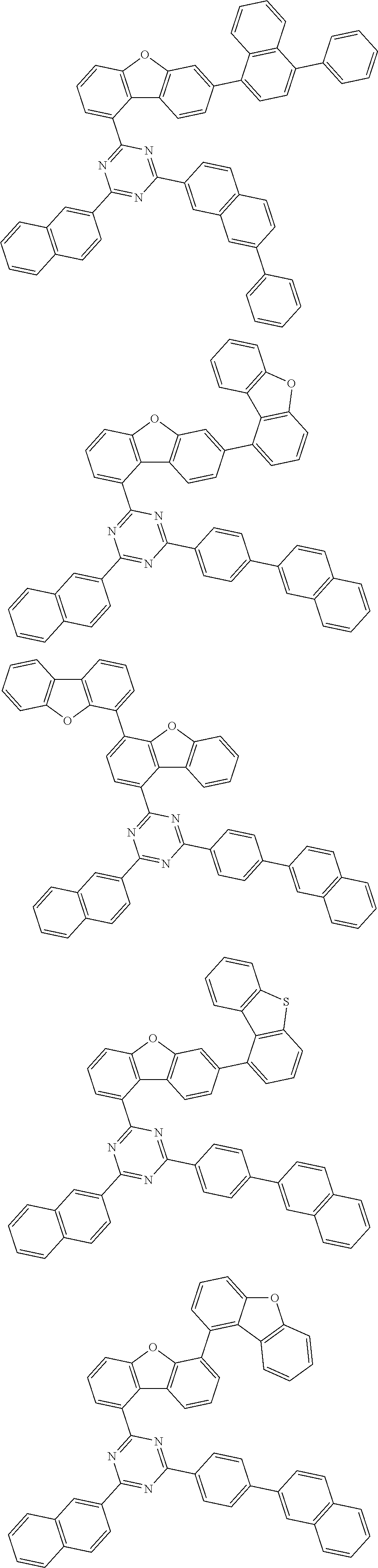

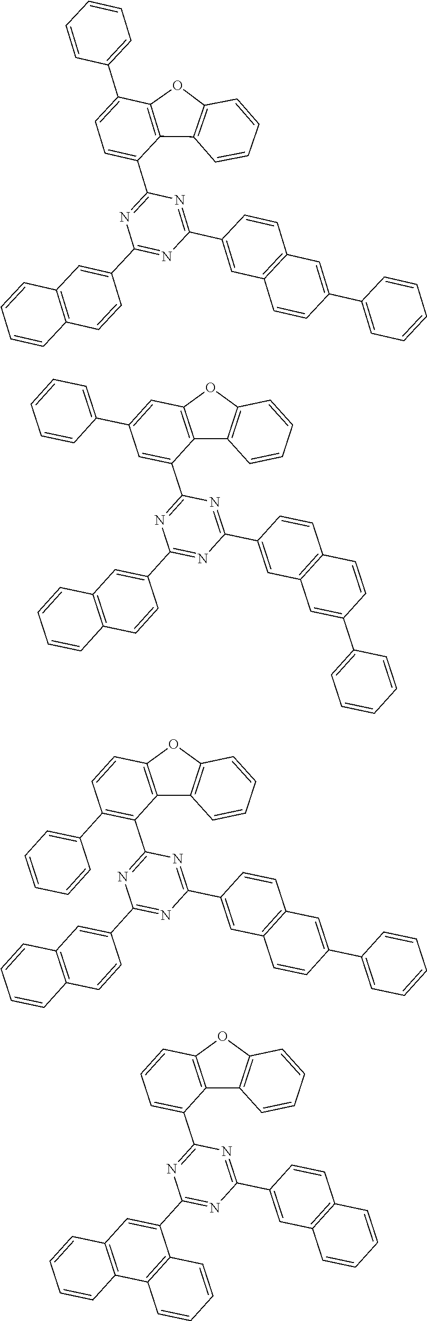

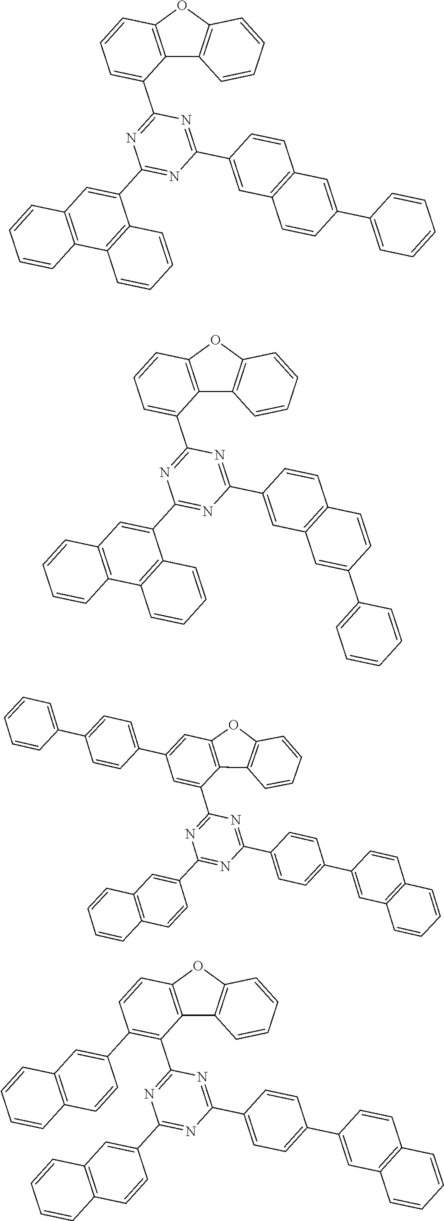

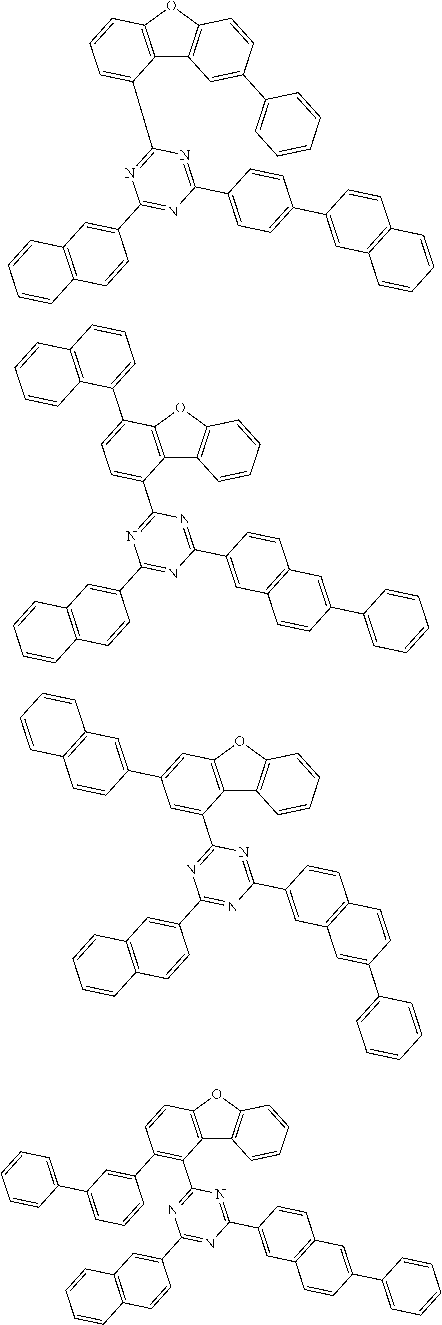

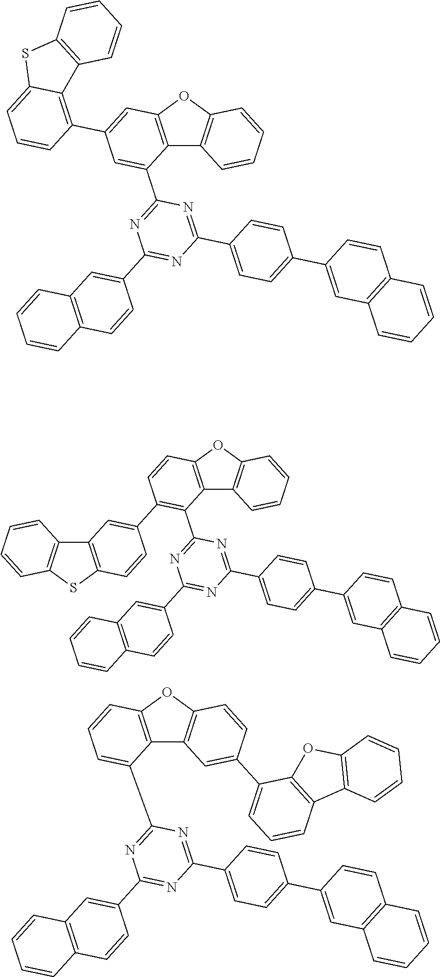

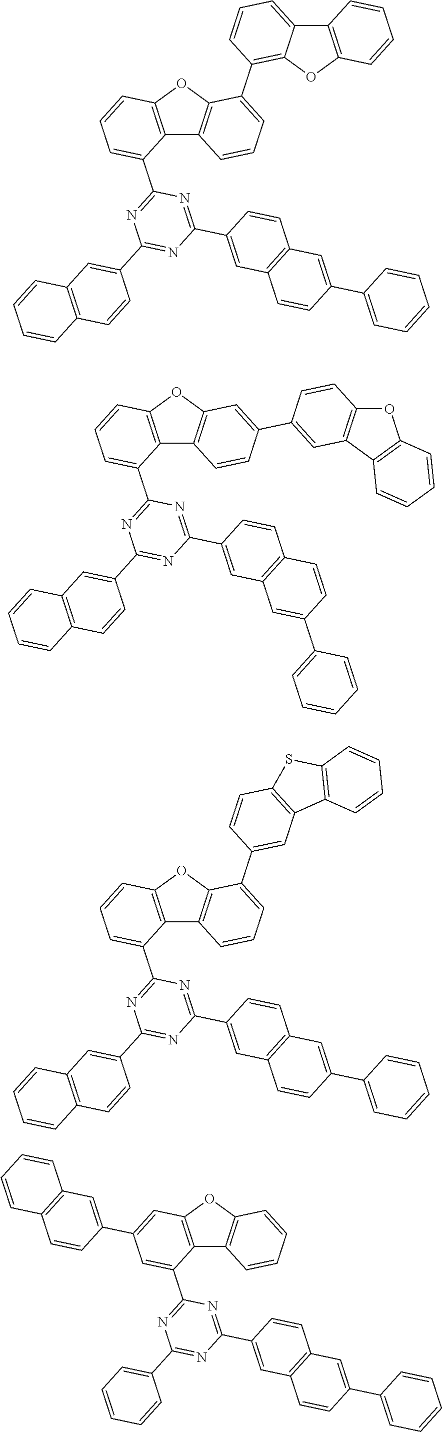

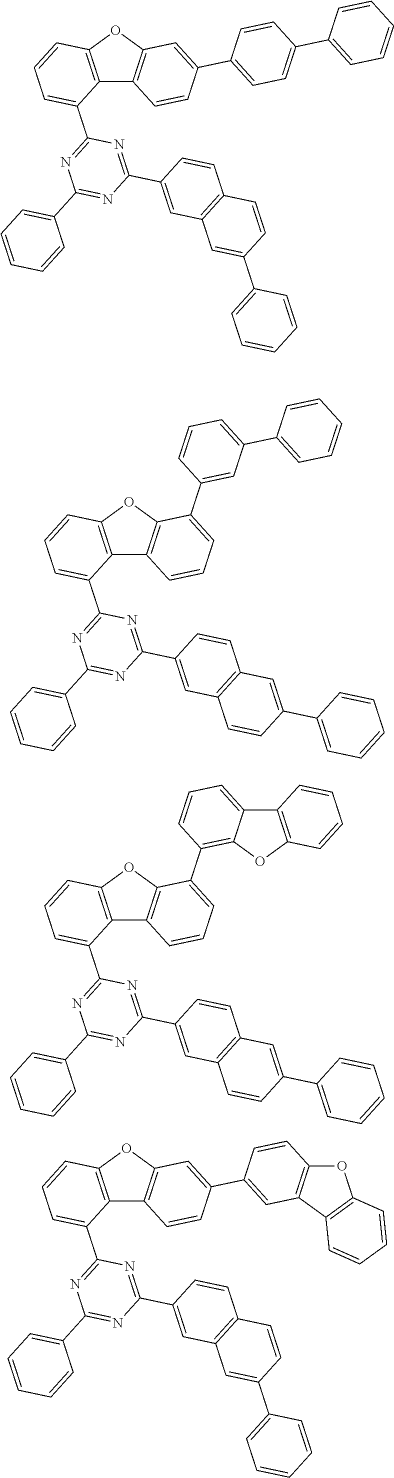

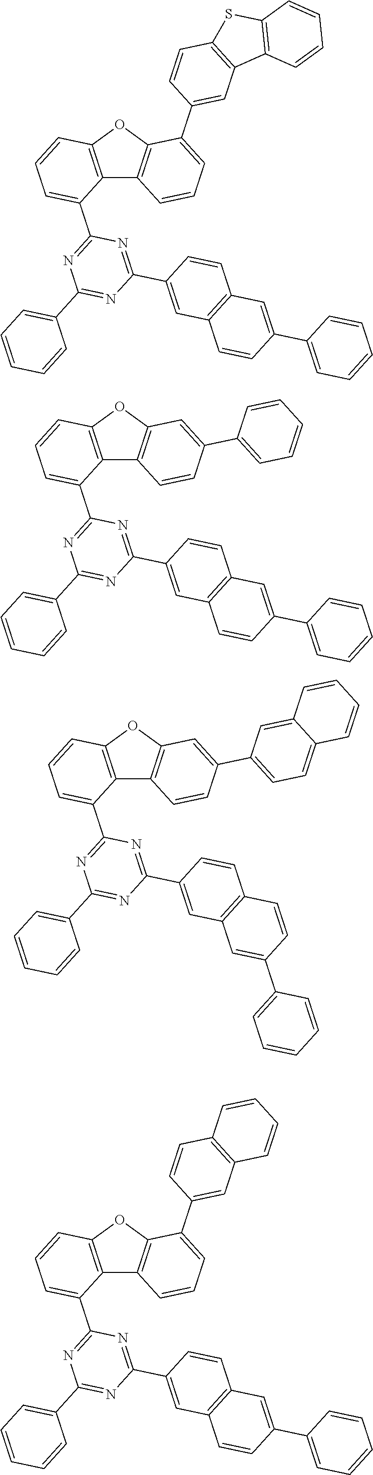

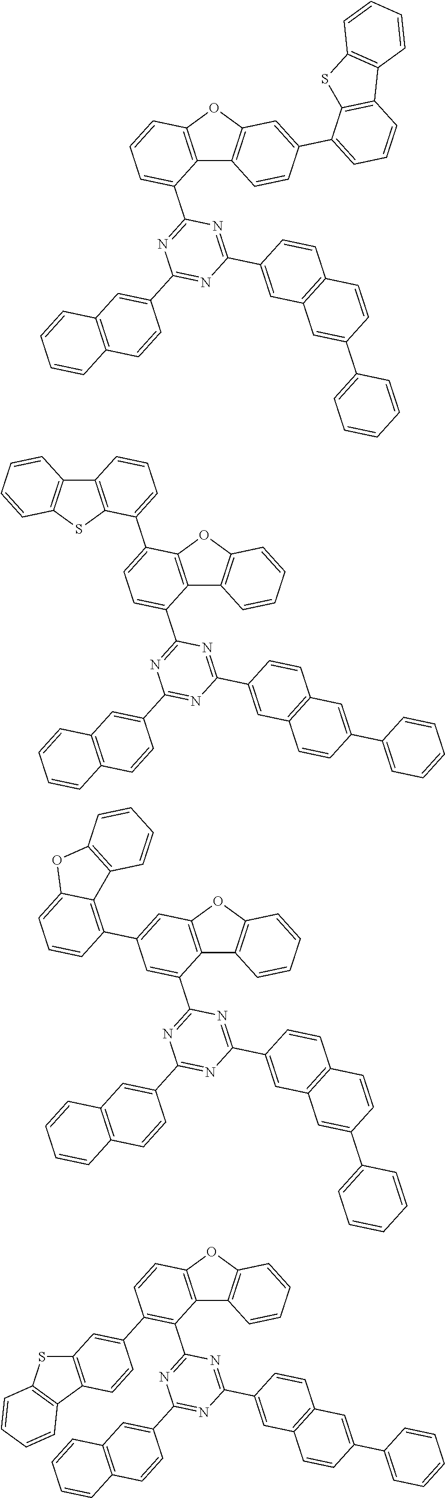

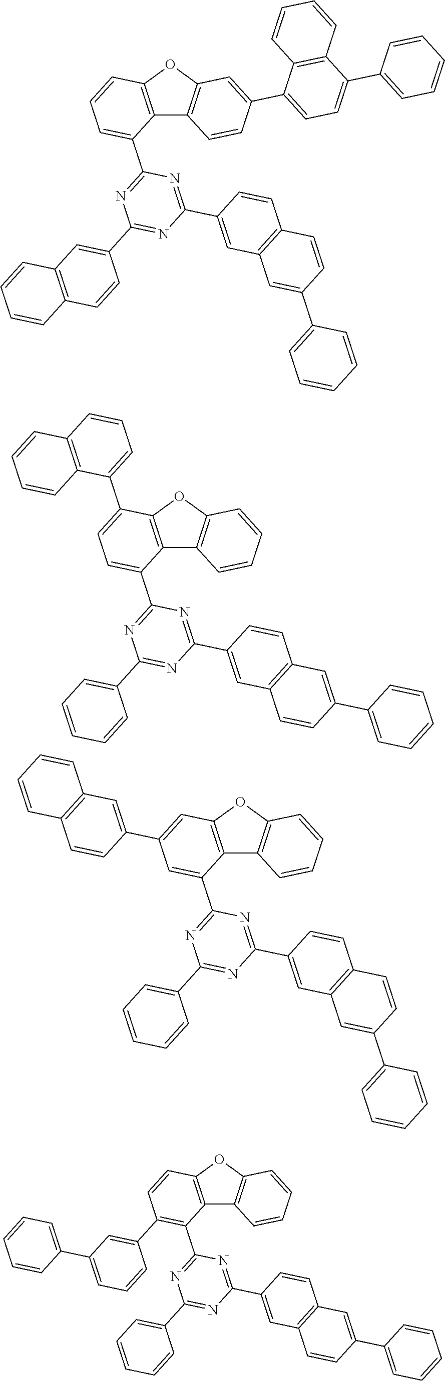

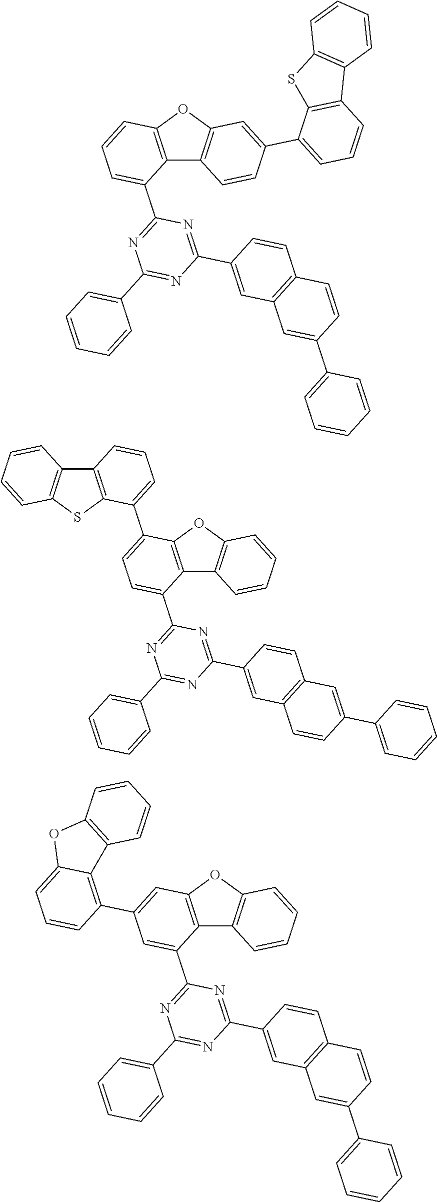

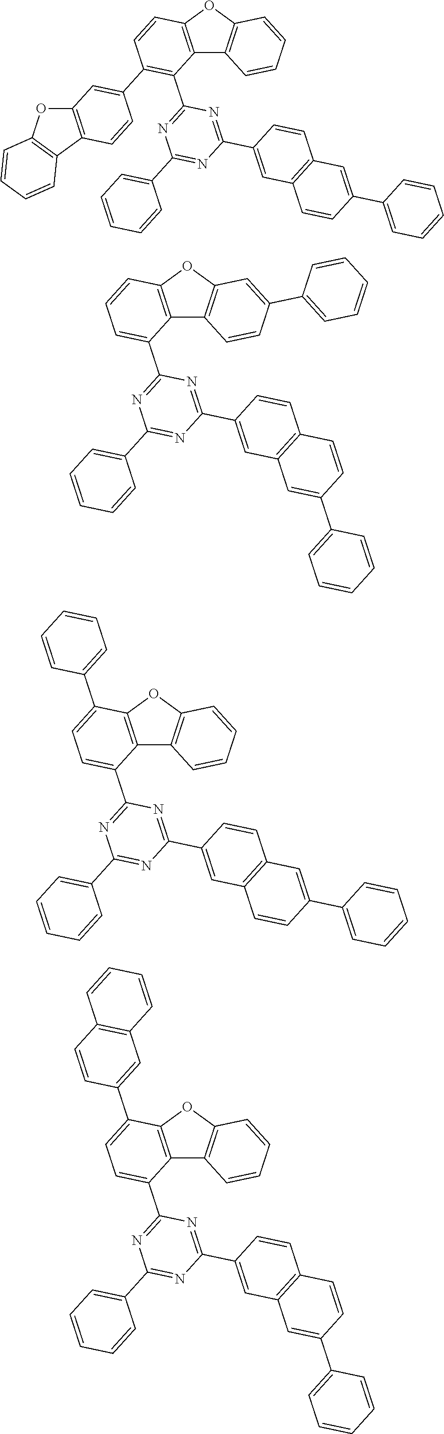

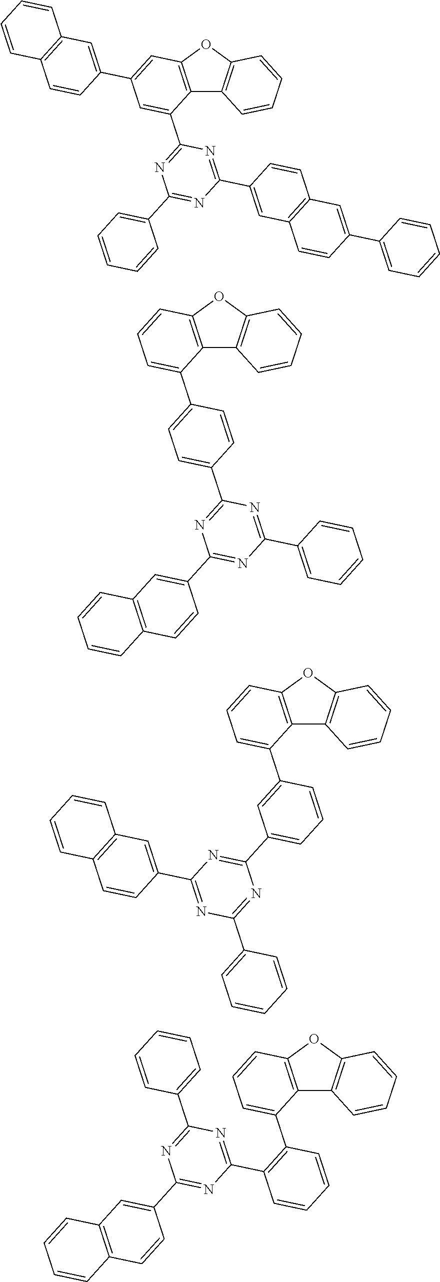

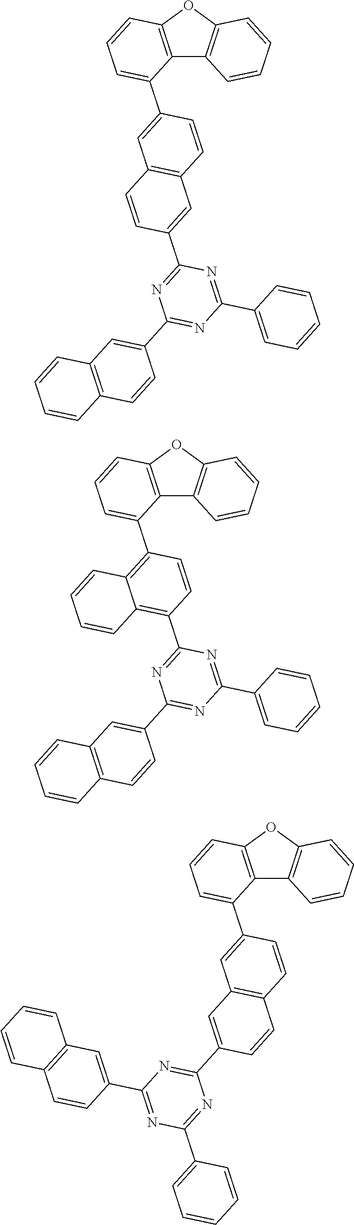 [SMILES: c1ccc(-c2nc(-c3ccc4ccccc4c3)nc(-c3ccc4cc(-c5cccc6oc7ccccc7c56)ccc4c3)n2)cc1 ; c1ccc(-c2nc(-c3ccc4ccccc4c3)nc(-c3ccc(-c4cccc5oc6ccccc6c45)c4ccccc34)n2)cc1 ; c1ccc(-c2nc(-c3ccc4ccccc4c3)nc(-c3ccc4ccc(-c5cccc6oc7ccccc7c56)cc4c3)n2)cc1]

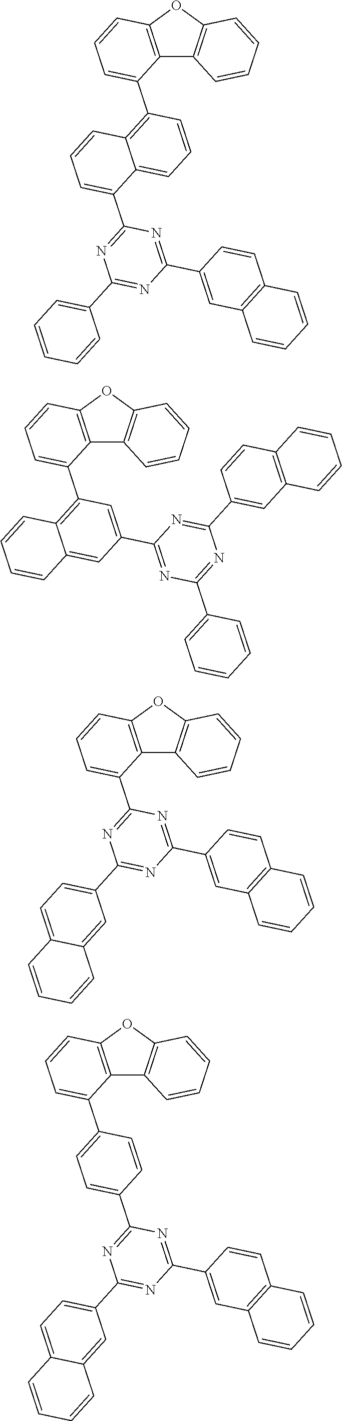

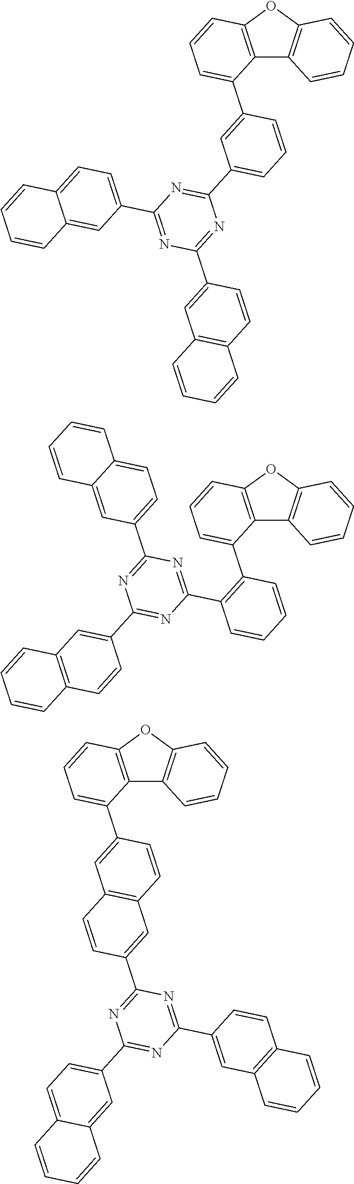 [SMILES: c1cc(-c2nc(-c3ccc4ccccc4c3)nc(-c3ccc4ccccc4c3)n2)cc(-c2cccc3oc4ccccc4c23)c1 ; c1ccc(-c2cccc3oc4ccccc4c23)c(-c2nc(-c3ccc4ccccc4c3)nc(-c3ccc4ccccc4c3)n2)c1 ; c1ccc2cc(-c3nc(-c4ccc5ccccc5c4)nc(-c4ccc5cc(-c6cccc7oc8ccccc8c67)ccc5c4)n3)ccc2c1]

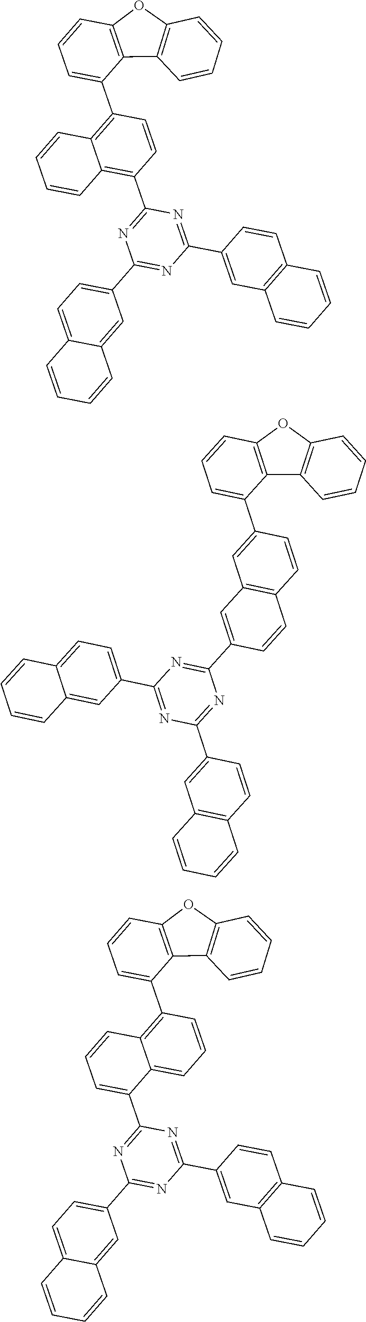

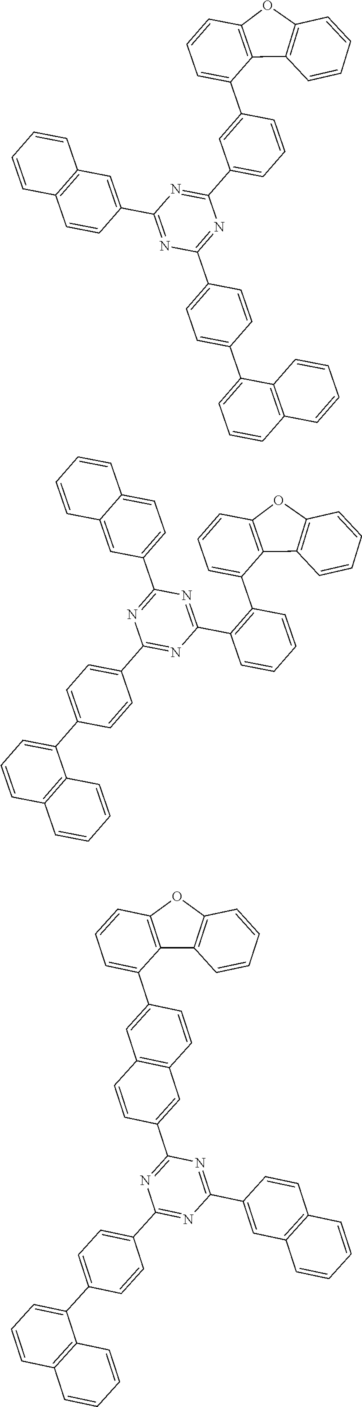 [SMILES: c1cc(-c2nc(-c3ccc(-c4cccc5ccccc45)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4cccc5ccccc45)cc3)nc(-c3ccc4ccccc4c3)n2)c1 ; c1ccc2cc(-c3nc(-c4ccc(-c5cccc6ccccc56)cc4)nc(-c4ccc5cc(-c6cccc7oc8ccccc8c67)ccc5c4)n3)ccc2c1]

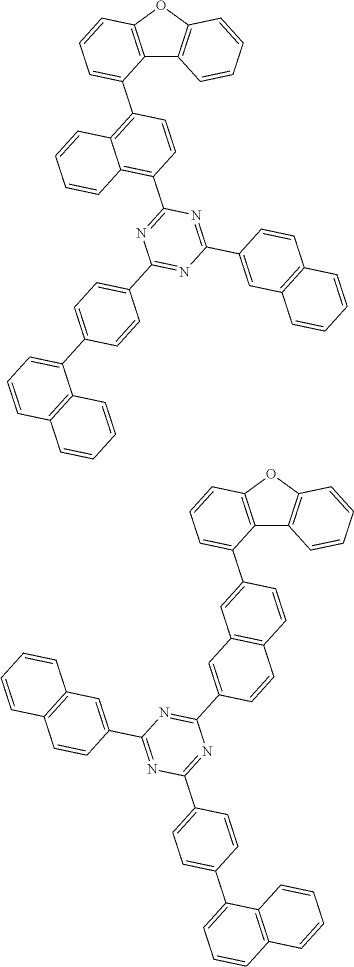 [SMILES: c1ccc2cc(-c3nc(-c4ccc(-c5cccc6ccccc56)cc4)nc(-c4ccc(-c5cccc6oc7ccccc7c56)c5ccccc45)n3)ccc2c1 ; c1ccc2cc(-c3nc(-c4ccc(-c5cccc6ccccc56)cc4)nc(-c4ccc5ccc(-c6cccc7oc8ccccc8c67)cc5c4)n3)ccc2c1]

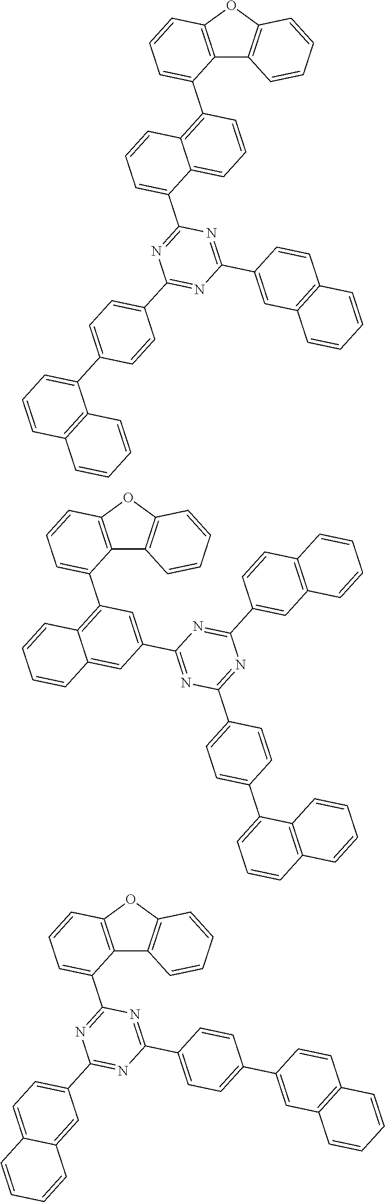

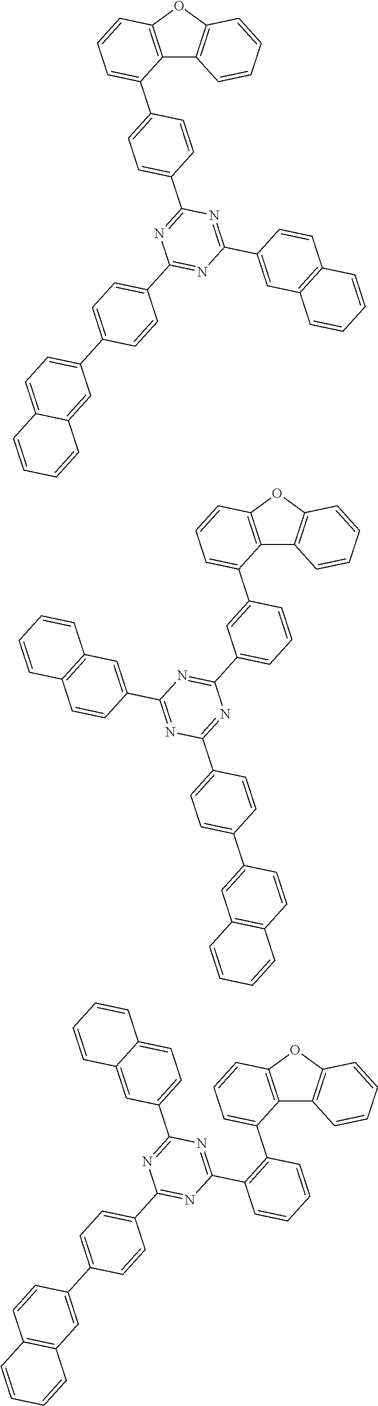 [SMILES: c1ccc2cc(-c3ccc(-c4nc(-c5ccc(-c6cccc7oc8ccccc8c67)cc5)nc(-c5ccc6ccccc6c5)n4)cc3)ccc2c1 ; c1cc(-c2nc(-c3ccc(-c4ccc5ccccc5c4)cc3)nc(-c3ccc4ccccc4c3)n2)cc(-c2cccc3oc4ccccc4c23)c1 ; c1ccc(-c2cccc3oc4ccccc4c23)c(-c2nc(-c3ccc(-c4ccc5ccccc5c4)cc3)nc(-c3ccc4ccccc4c3)n2)c1]

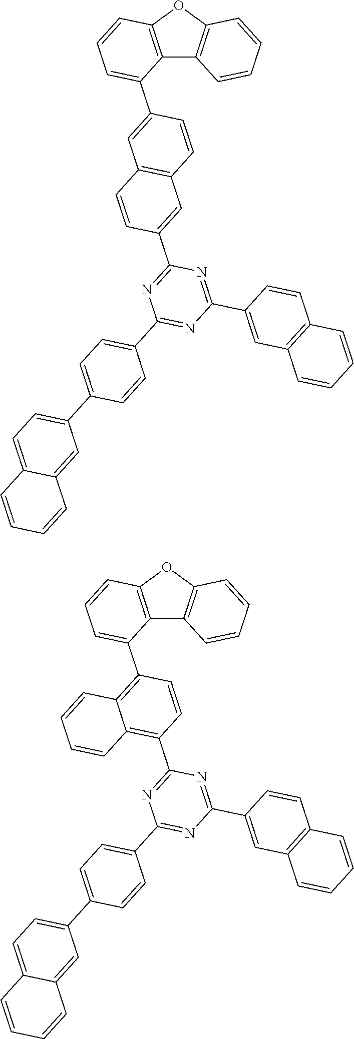 [SMILES: c1ccc2cc(-c3ccc(-c4nc(-c5ccc6ccccc6c5)nc(-c5ccc6cc(-c7cccc8oc9ccccc9c78)ccc6c5)n4)cc3)ccc2c1 ; c1ccc2cc(-c3ccc(-c4nc(-c5ccc6ccccc6c5)nc(-c5ccc(-c6cccc7oc8ccccc8c67)c6ccccc56)n4)cc3)ccc2c1]

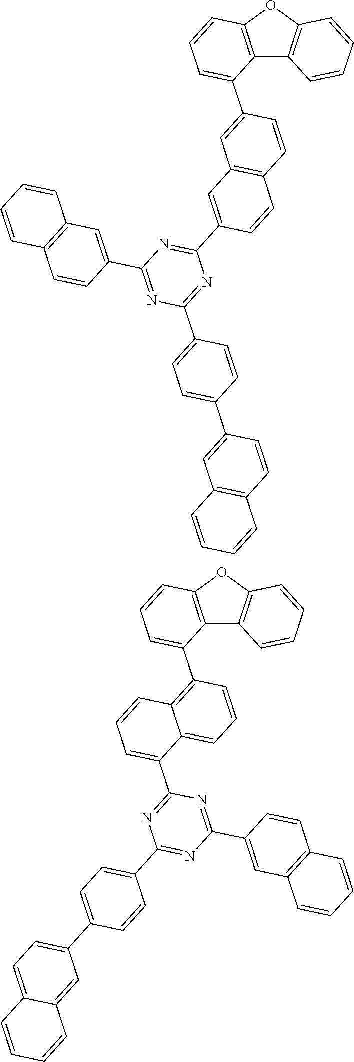

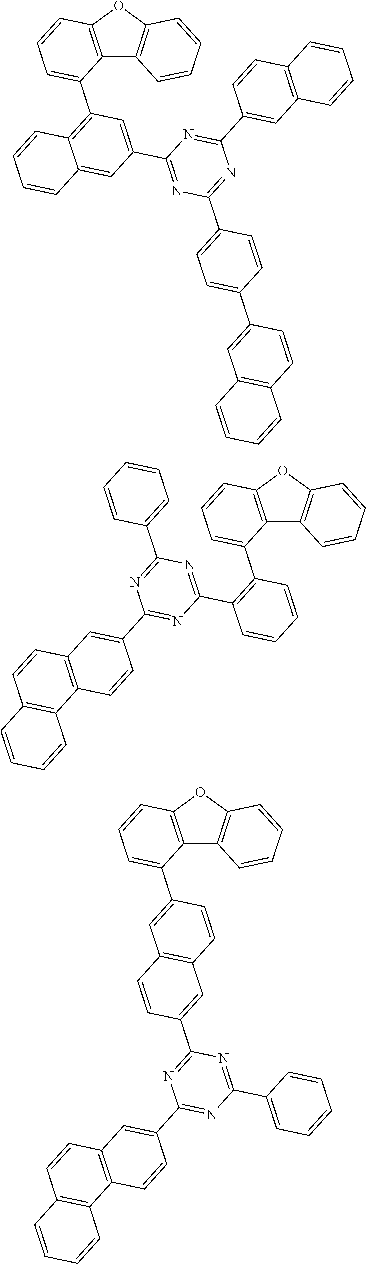

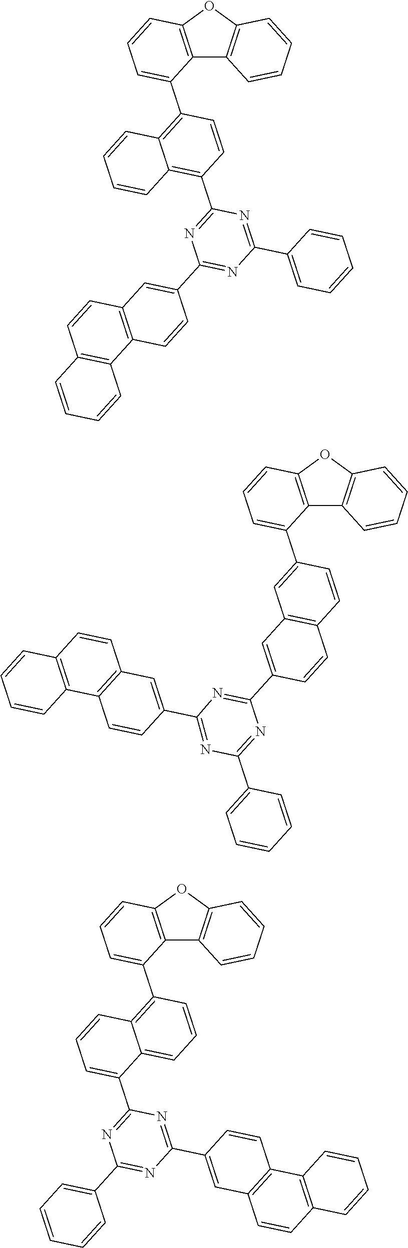 [SMILES: c1ccc(-c2nc(-c3ccc4c(ccc5ccccc54)c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(ccc5ccccc54)c3)n2)cc1 ; c1ccc(-c2nc(-c3ccc4c(ccc5ccccc54)c3)nc(-c3cccc4c(-c5cccc6oc7ccccc7c56)cccc34)n2)cc1]

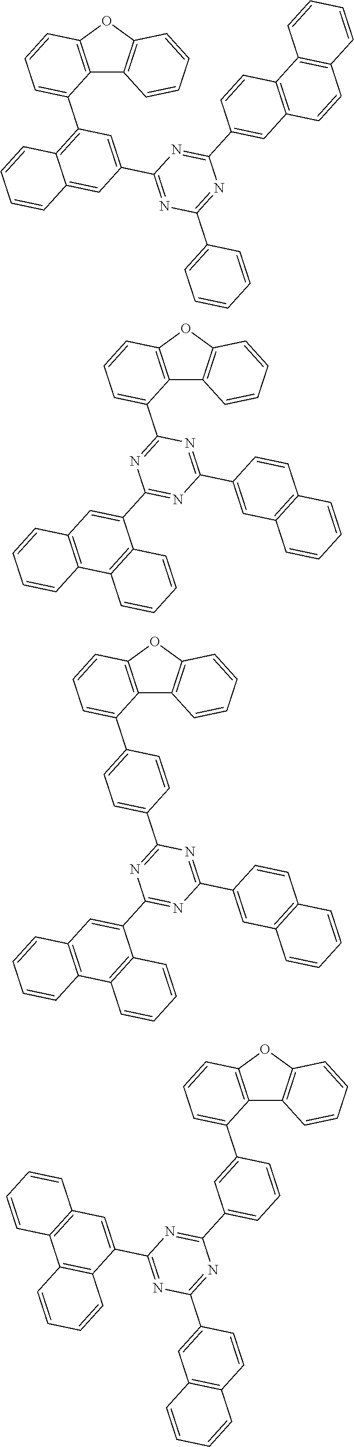

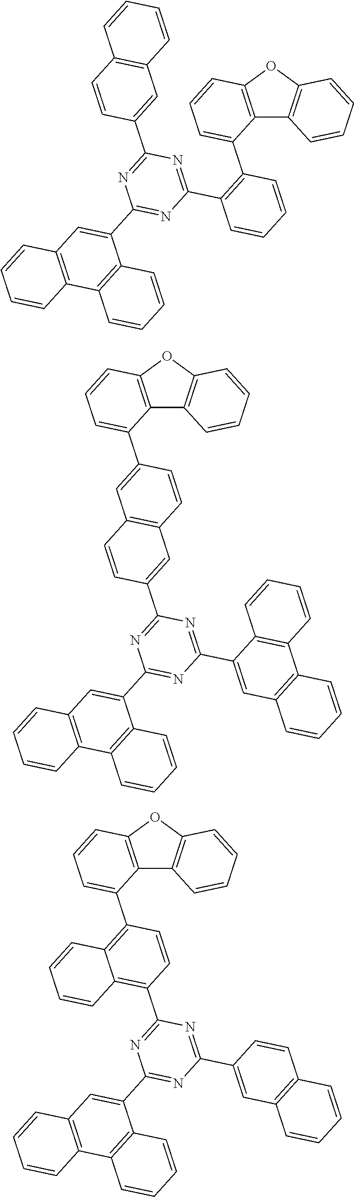

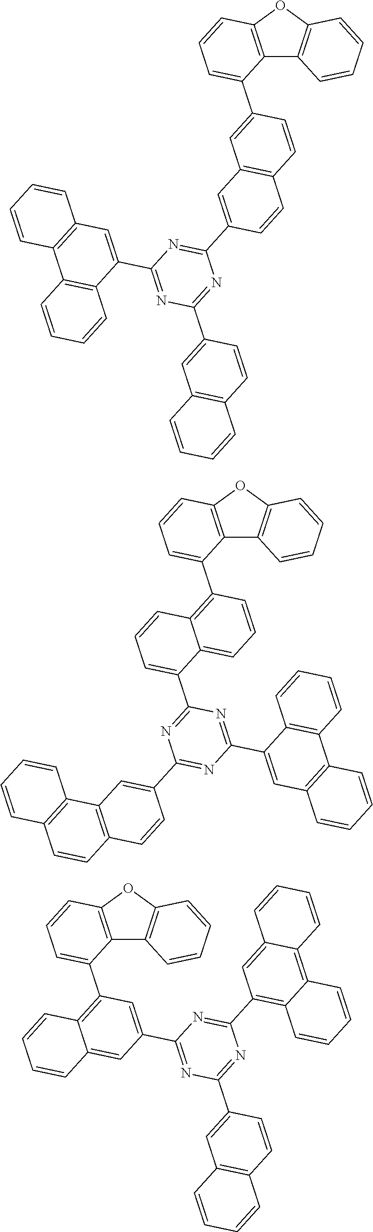

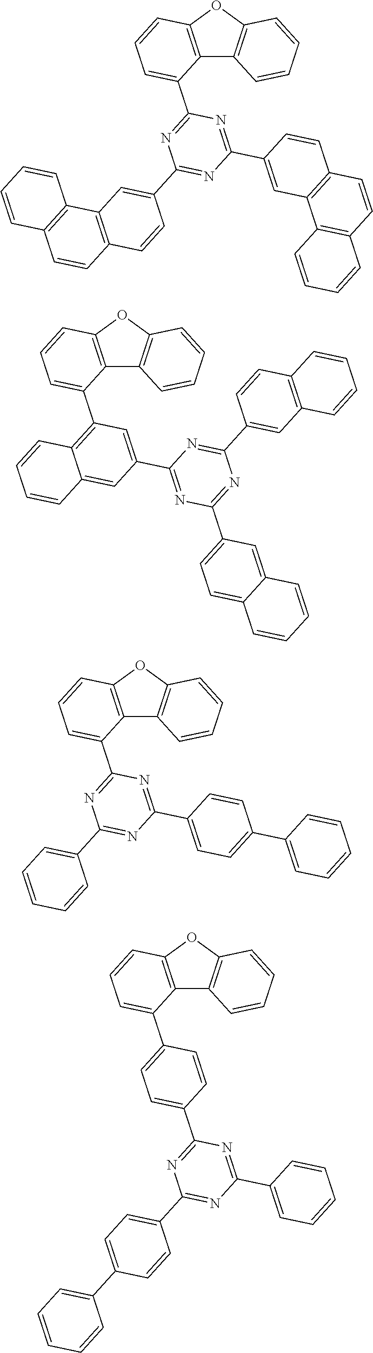 [SMILES: c1ccc2c(c1)ccc1ccc(-c3nc(-c4ccc5ccc6ccccc6c5c4)nc(-c4cccc5oc6ccccc6c45)n3)cc12 ; c1ccc2cc(-c3nc(-c4ccc5ccccc5c4)nc(-c4cc(-c5cccc6oc7ccccc7c56)c5ccccc5c4)n3)ccc2c1 ; c1ccc(-c2ccc(-c3nc(-c4ccccc4)nc(-c4cccc5oc6ccccc6c45)n3)cc2)cc1 ; c1ccc(-c2ccc(-c3nc(-c4ccccc4)nc(-c4ccc(-c5cccc6oc7ccccc7c56)cc4)n3)cc2)cc1]

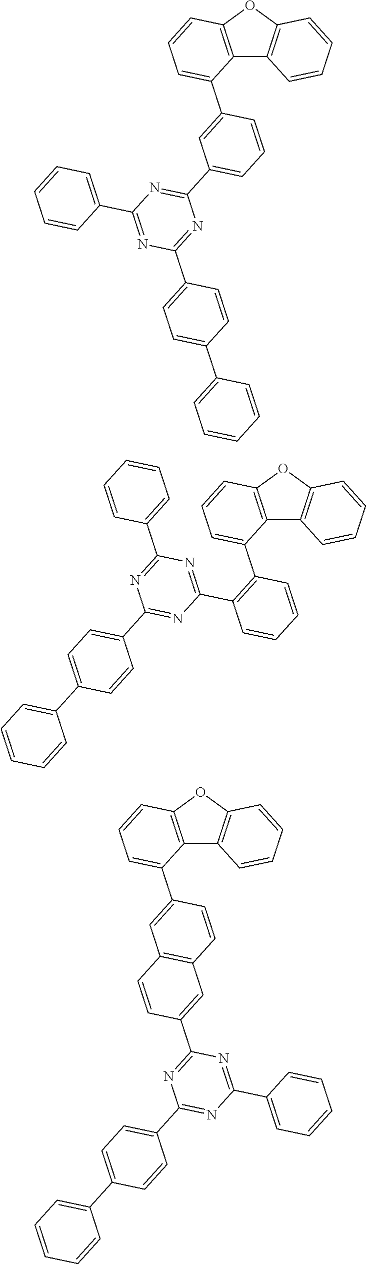

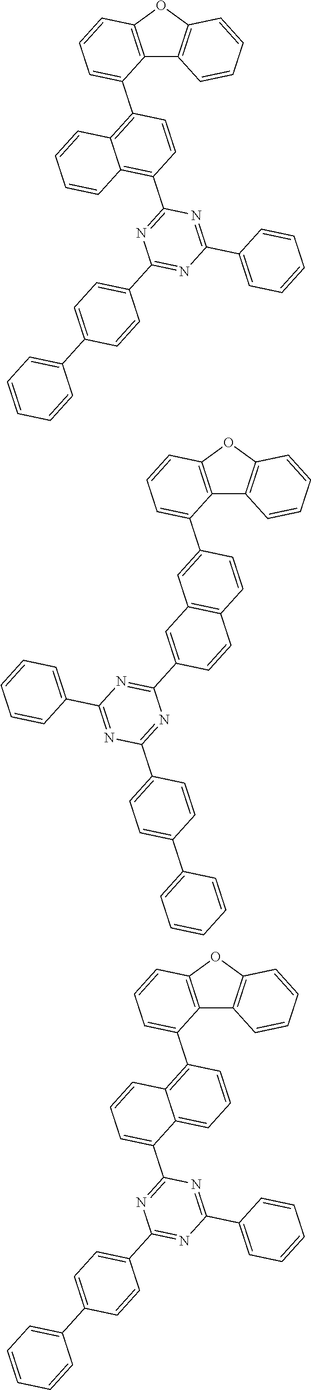

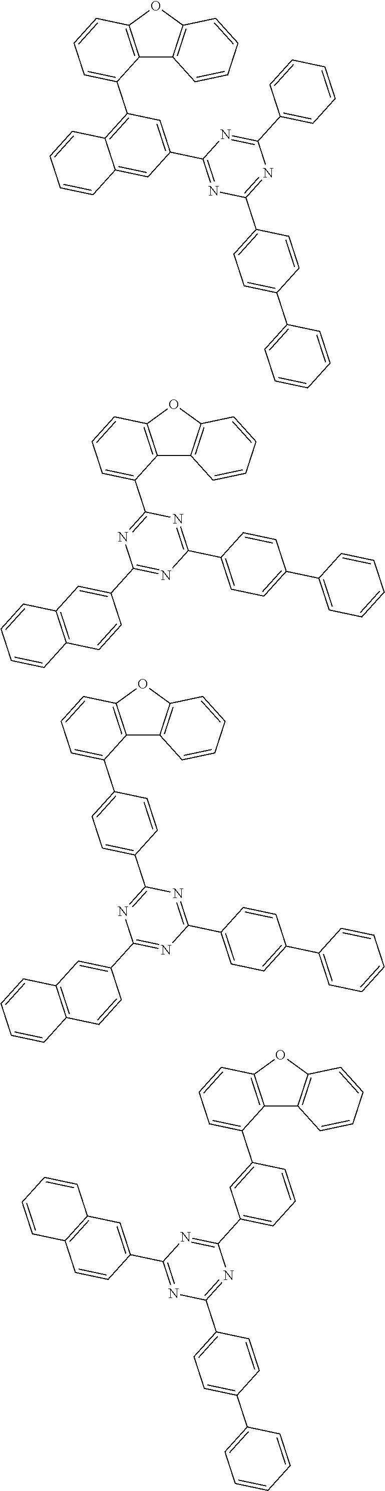 [SMILES: c1ccc(-c2ccc(-c3nc(-c4ccccc4)nc(-c4cc(-c5cccc6oc7ccccc7c56)c5ccccc5c4)n3)cc2)cc1 ; c1ccc(-c2ccc(-c3nc(-c4ccc5ccccc5c4)nc(-c4cccc5oc6ccccc6c45)n3)cc2)cc1 ; c1ccc(-c2ccc(-c3nc(-c4ccc(-c5cccc6oc7ccccc7c56)cc4)nc(-c4ccc5ccccc5c4)n3)cc2)cc1 ; c1ccc(-c2ccc(-c3nc(-c4cccc(-c5cccc6oc7ccccc7c56)c4)nc(-c4ccc5ccccc5c4)n3)cc2)cc1]

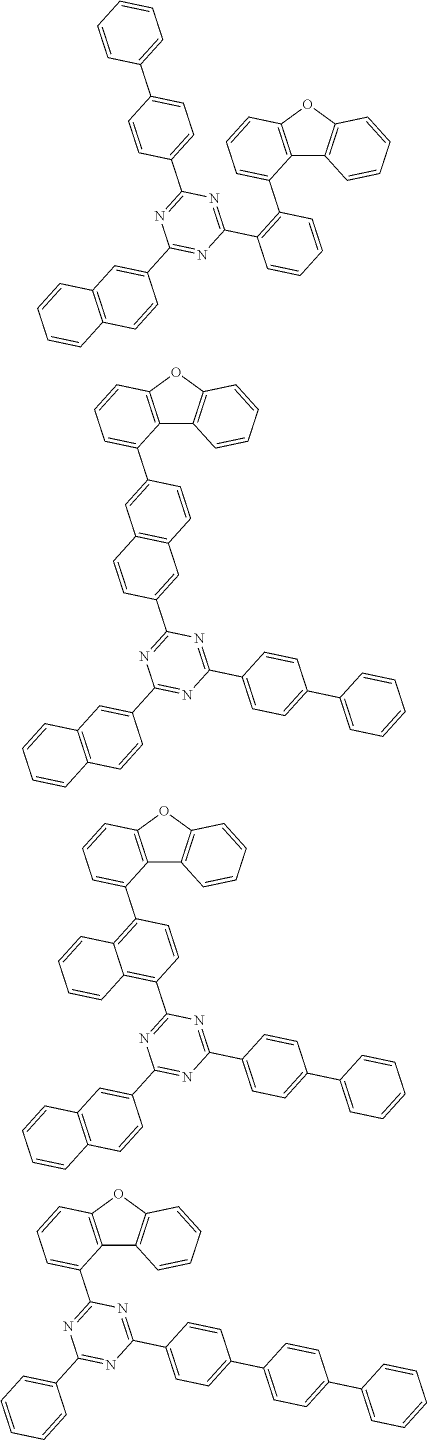

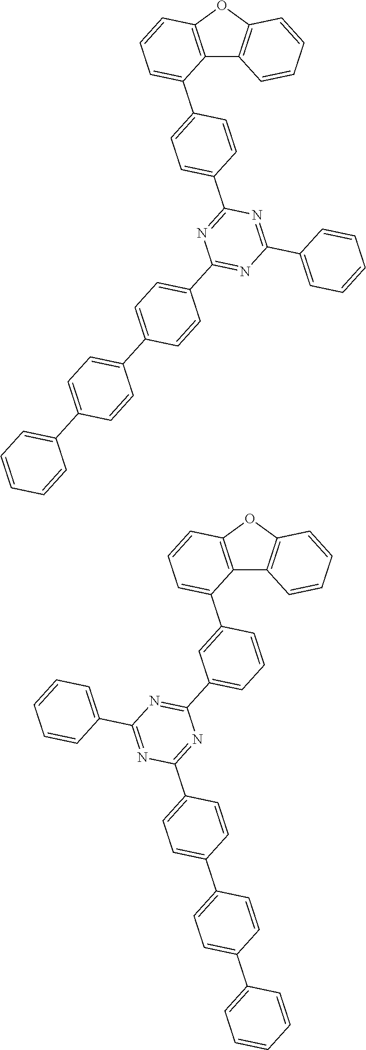 [SMILES: c1ccc(-c2ccc(-c3ccc(-c4nc(-c5ccccc5)nc(-c5ccc(-c6cccc7oc8ccccc8c67)cc5)n4)cc3)cc2)cc1 ; c1ccc(-c2ccc(-c3ccc(-c4nc(-c5ccccc5)nc(-c5cccc(-c6cccc7oc8ccccc8c67)c5)n4)cc3)cc2)cc1]

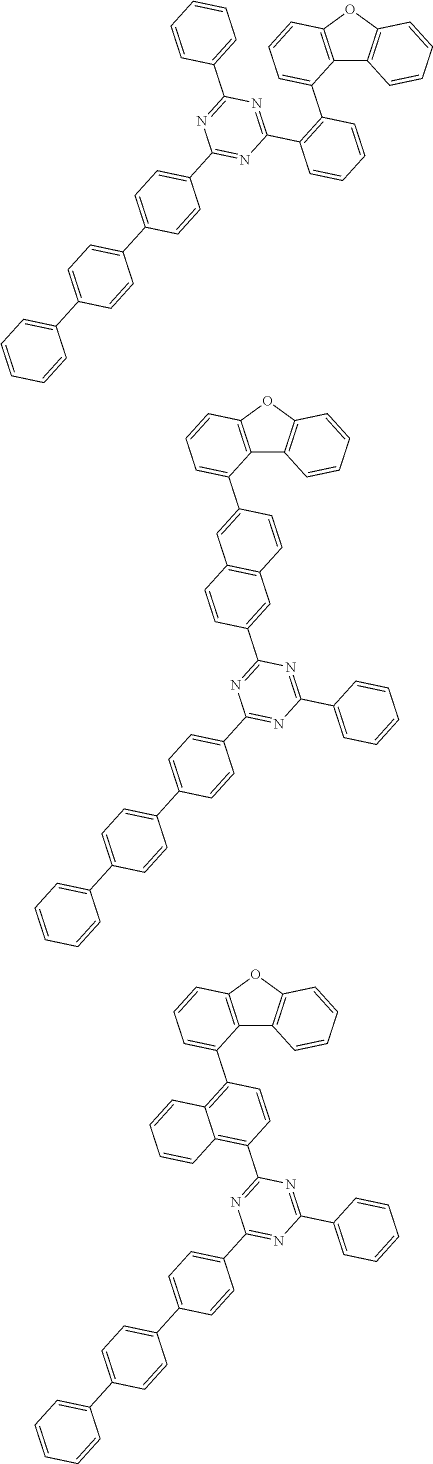 [SMILES: c1ccc(-c2ccc(-c3ccc(-c4nc(-c5ccccc5)nc(-c5ccccc5-c5cccc6oc7ccccc7c56)n4)cc3)cc2)cc1 ; c1ccc(-c2ccc(-c3ccc(-c4nc(-c5ccccc5)nc(-c5ccc6cc(-c7cccc8oc9ccccc9c78)ccc6c5)n4)cc3)cc2)cc1 ; c1ccc(-c2ccc(-c3ccc(-c4nc(-c5ccccc5)nc(-c5ccc(-c6cccc7oc8ccccc8c67)c6ccccc56)n4)cc3)cc2)cc1]

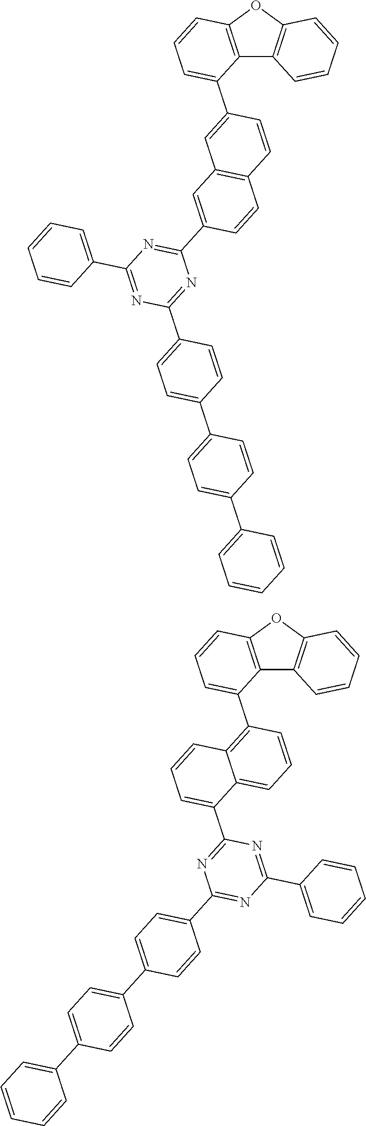

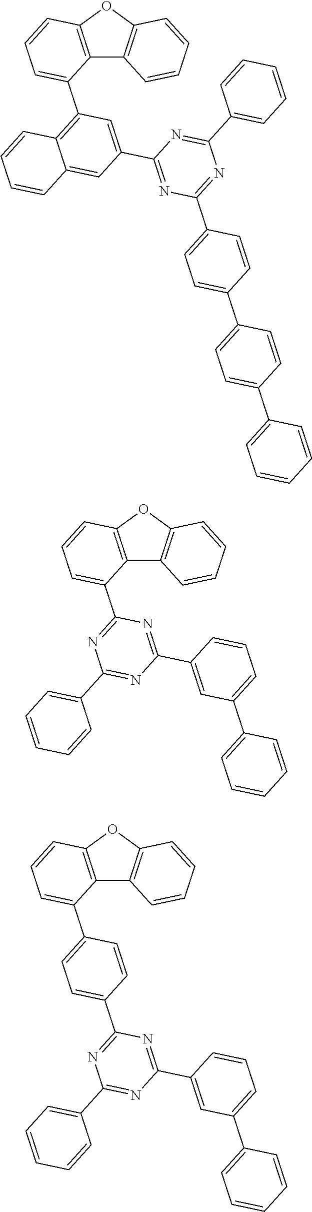 [SMILES: c1ccc(-c2ccc(-c3ccc(-c4nc(-c5ccccc5)nc(-c5cc(-c6cccc7oc8ccccc8c67)c6ccccc6c5)n4)cc3)cc2)cc1 ; c1ccc(-c2cccc(-c3nc(-c4ccccc4)nc(-c4cccc5oc6ccccc6c45)n3)c2)cc1 ; c1ccc(-c2cccc(-c3nc(-c4ccccc4)nc(-c4ccc(-c5cccc6oc7ccccc7c56)cc4)n3)c2)cc1]

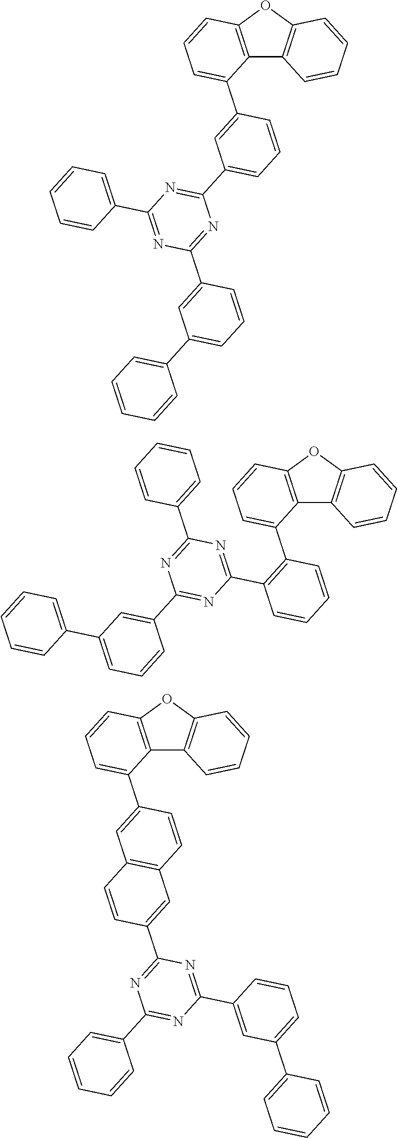

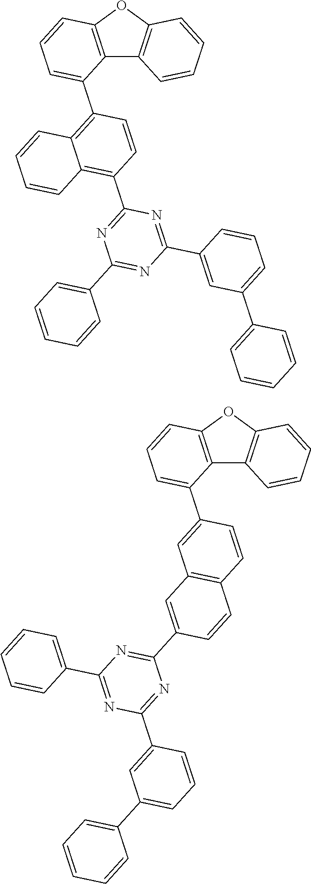

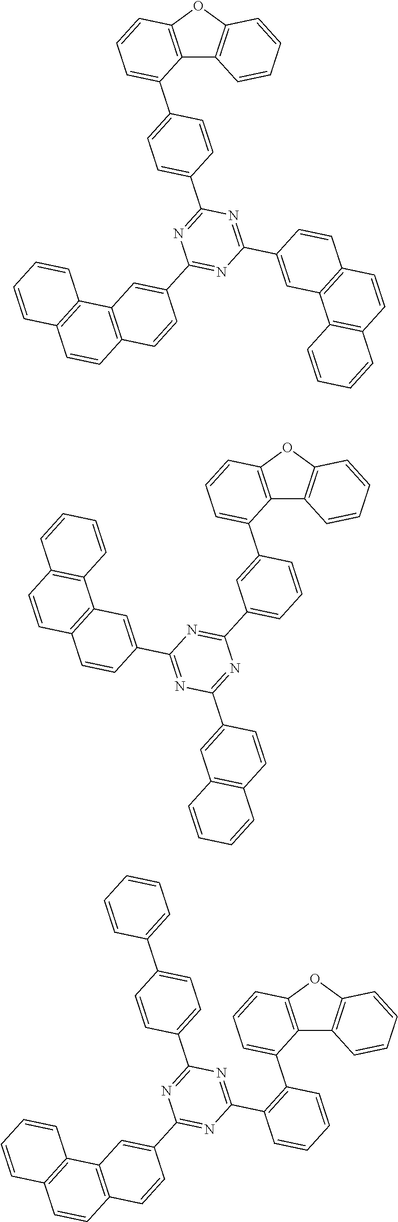 [SMILES: c1ccc2c(c1)ccc1ccc(-c3nc(-c4ccc(-c5cccc6oc7ccccc7c56)cc4)nc(-c4ccc5ccc6ccccc6c5c4)n3)cc12 ; c1cc(-c2nc(-c3ccc4ccccc4c3)nc(-c3ccc4ccc5ccccc5c4c3)n2)cc(-c2cccc3oc4ccccc4c23)c1 ; c1ccc(-c2ccc(-c3nc(-c4ccc5ccc6ccccc6c5c4)nc(-c4ccccc4-c4cccc5oc6ccccc6c45)n3)cc2)cc1]

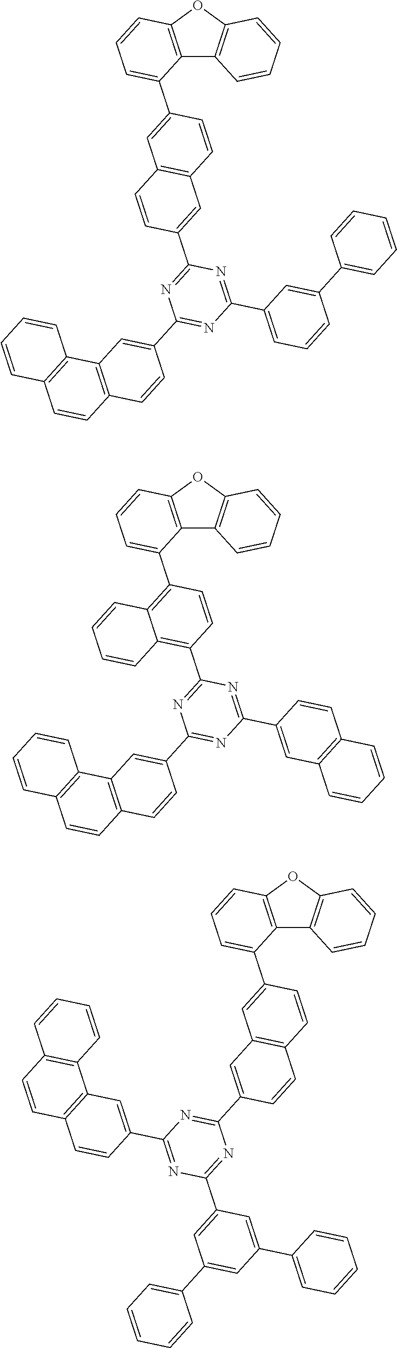 [SMILES: c1ccc(-c2cccc(-c3nc(-c4ccc5cc(-c6cccc7oc8ccccc8c67)ccc5c4)nc(-c4ccc5ccc6ccccc6c5c4)n3)c2)cc1 ; c1ccc2cc(-c3nc(-c4ccc5ccc6ccccc6c5c4)nc(-c4ccc(-c5cccc6oc7ccccc7c56)c5ccccc45)n3)ccc2c1 ; c1ccc(-c2cc(-c3ccccc3)cc(-c3nc(-c4ccc5ccc(-c6cccc7oc8ccccc8c67)cc5c4)nc(-c4ccc5ccc6ccccc6c5c4)n3)c2)cc1]

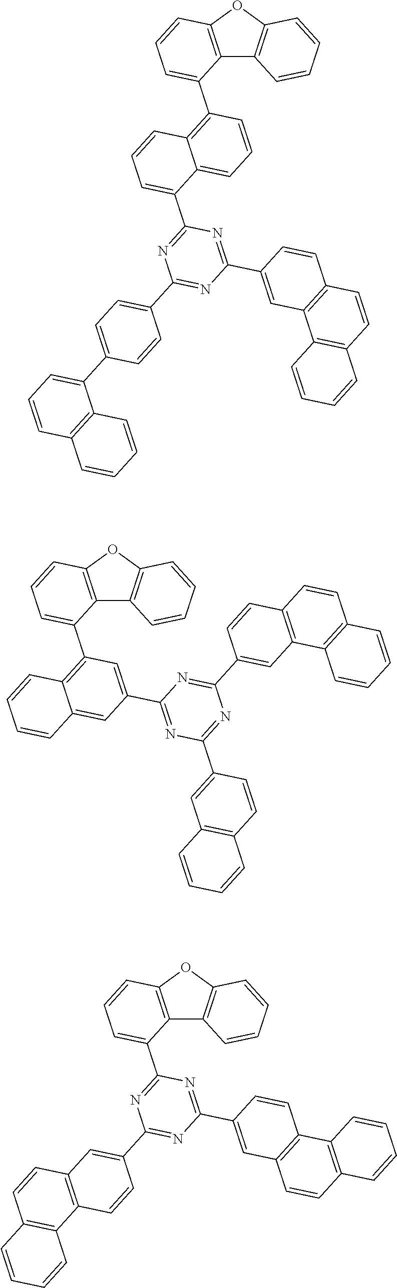 [SMILES: c1ccc2c(-c3ccc(-c4nc(-c5ccc6ccc7ccccc7c6c5)nc(-c5cccc6c(-c7cccc8oc9ccccc9c78)cccc56)n4)cc3)cccc2c1 ; c1ccc2cc(-c3nc(-c4cc(-c5cccc6oc7ccccc7c56)c5ccccc5c4)nc(-c4ccc5ccc6ccccc6c5c4)n3)ccc2c1 ; c1ccc2c(c1)ccc1cc(-c3nc(-c4ccc5c(ccc6ccccc65)c4)nc(-c4cccc5oc6ccccc6c45)n3)ccc12]

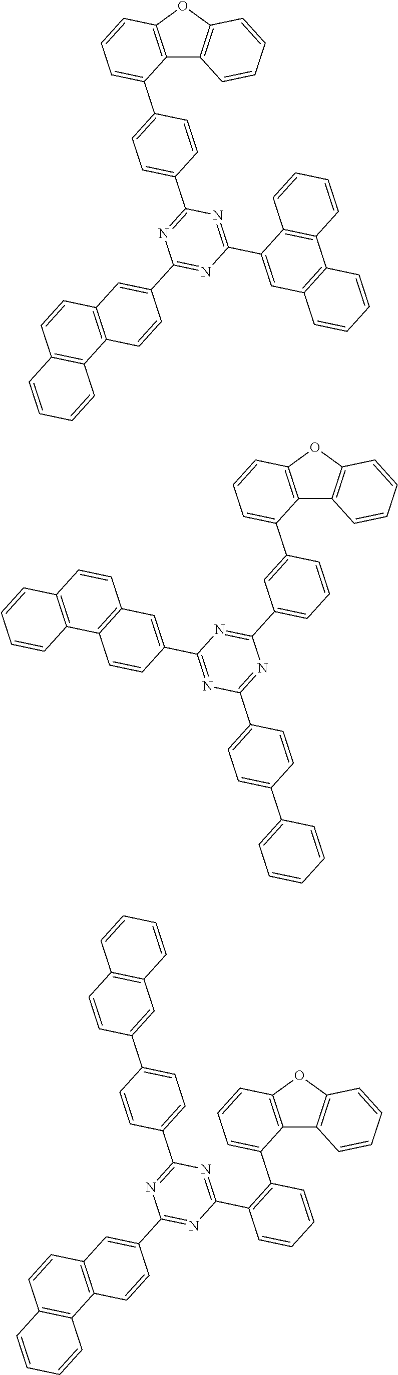 [SMILES: c1ccc2c(c1)ccc1cc(-c3nc(-c4ccc(-c5cccc6oc7ccccc7c56)cc4)nc(-c4cc5ccccc5c5ccccc45)n3)ccc12 ; c1ccc(-c2ccc(-c3nc(-c4cccc(-c5cccc6oc7ccccc7c56)c4)nc(-c4ccc5c(ccc6ccccc65)c4)n3)cc2)cc1 ; c1ccc(-c2cccc3oc4ccccc4c23)c(-c2nc(-c3ccc(-c4ccc5ccccc5c4)cc3)nc(-c3ccc4c(ccc5ccccc54)c3)n2)c1]

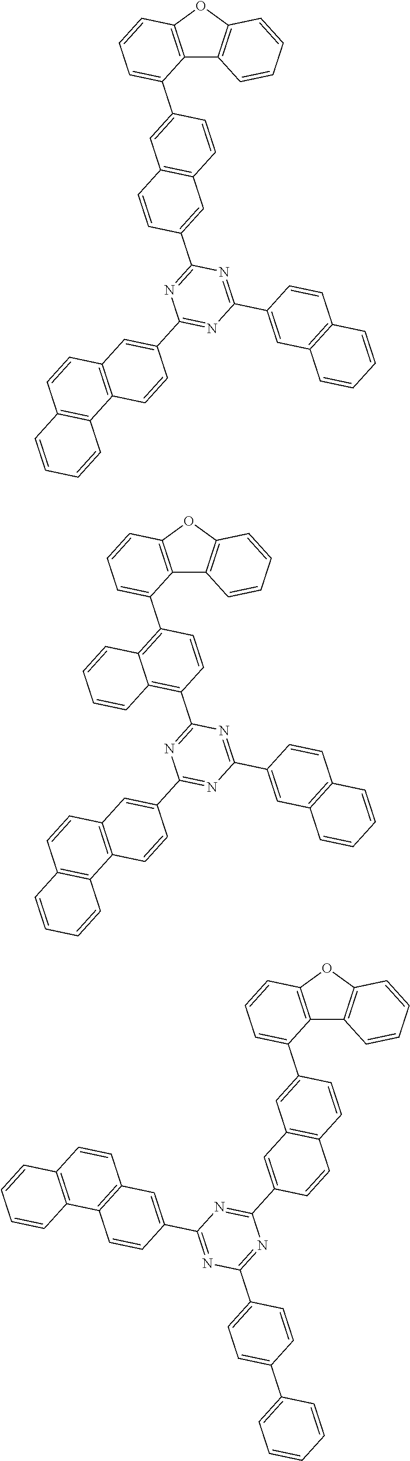 [SMILES: c1ccc2cc(-c3nc(-c4ccc5cc(-c6cccc7oc8ccccc8c67)ccc5c4)nc(-c4ccc5c(ccc6ccccc65)c4)n3)ccc2c1 ; c1ccc2cc(-c3nc(-c4ccc5c(ccc6ccccc65)c4)nc(-c4ccc(-c5cccc6oc7ccccc7c56)c5ccccc45)n3)ccc2c1 ; c1ccc(-c2ccc(-c3nc(-c4ccc5ccc(-c6cccc7oc8ccccc8c67)cc5c4)nc(-c4ccc5c(ccc6ccccc65)c4)n3)cc2)cc1]

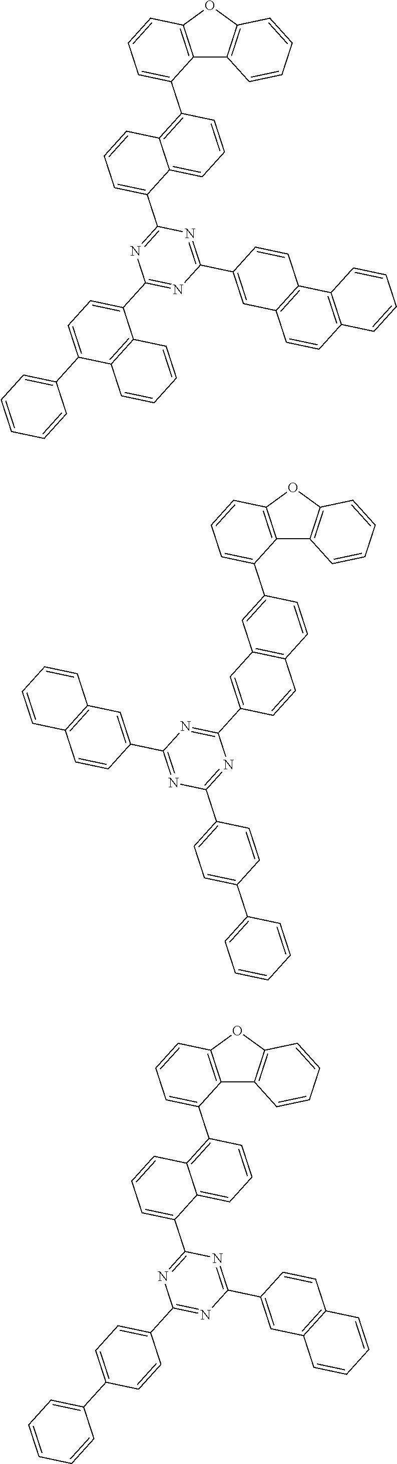 [SMILES: c1ccc(-c2ccc(-c3nc(-c4ccc5c(ccc6ccccc65)c4)nc(-c4cccc5c(-c6cccc7oc8ccccc8c67)cccc45)n3)c3ccccc23)cc1 ; c1ccc(-c2ccc(-c3nc(-c4ccc5ccccc5c4)nc(-c4ccc5ccc(-c6cccc7oc8ccccc8c67)cc5c4)n3)cc2)cc1 ; c1ccc(-c2ccc(-c3nc(-c4ccc5ccccc5c4)nc(-c4cccc5c(-c6cccc7oc8ccccc8c67)cccc45)n3)cc2)cc1]

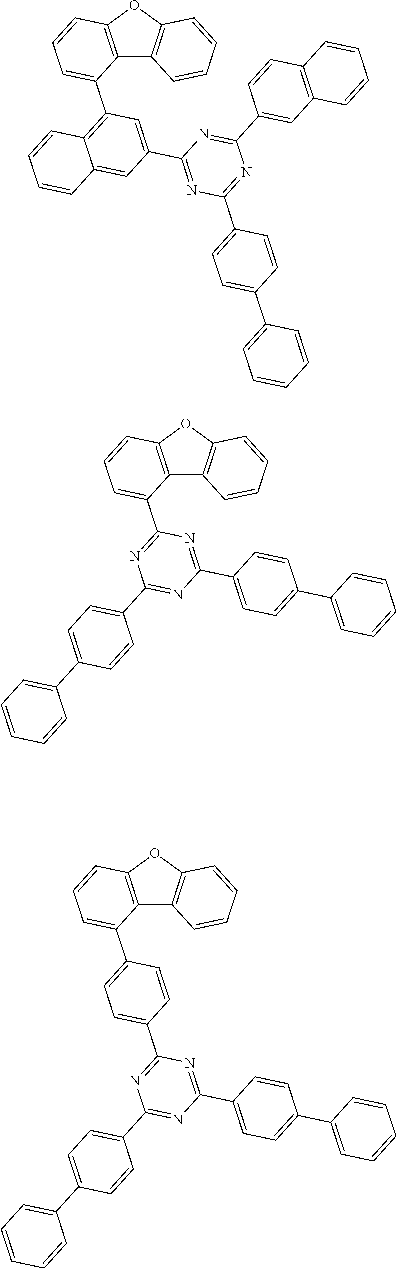 [SMILES: c1ccc(-c2ccc(-c3nc(-c4ccc5ccccc5c4)nc(-c4cc(-c5cccc6oc7ccccc7c56)c5ccccc5c4)n3)cc2)cc1 ; c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4ccc(-c5ccccc5)cc4)nc(-c4ccc(-c5cccc6oc7ccccc7c56)cc4)n3)cc2)cc1]

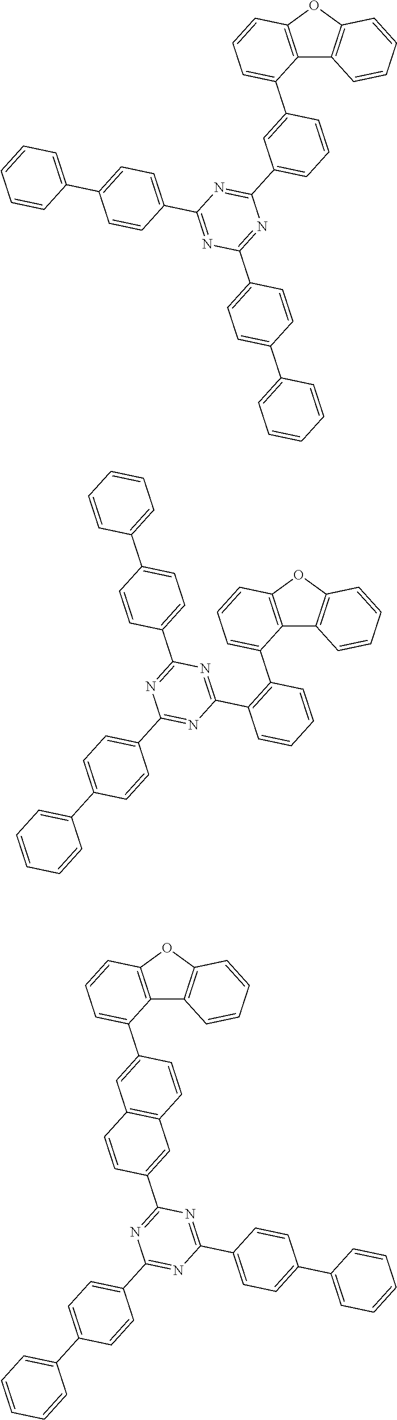 [SMILES: c1ccc(-c2ccc(-c3nc(-c4ccc(-c5ccccc5)cc4)nc(-c4cccc(-c5cccc6oc7ccccc7c56)c4)n3)cc2)cc1 ; c1ccc(-c2ccc(-c3nc(-c4ccc(-c5ccccc5)cc4)nc(-c4ccccc4-c4cccc5oc6ccccc6c45)n3)cc2)cc1 ; c1ccc(-c2ccc(-c3nc(-c4ccc(-c5ccccc5)cc4)nc(-c4ccc5cc(-c6cccc7oc8ccccc8c67)ccc5c4)n3)cc2)cc1]

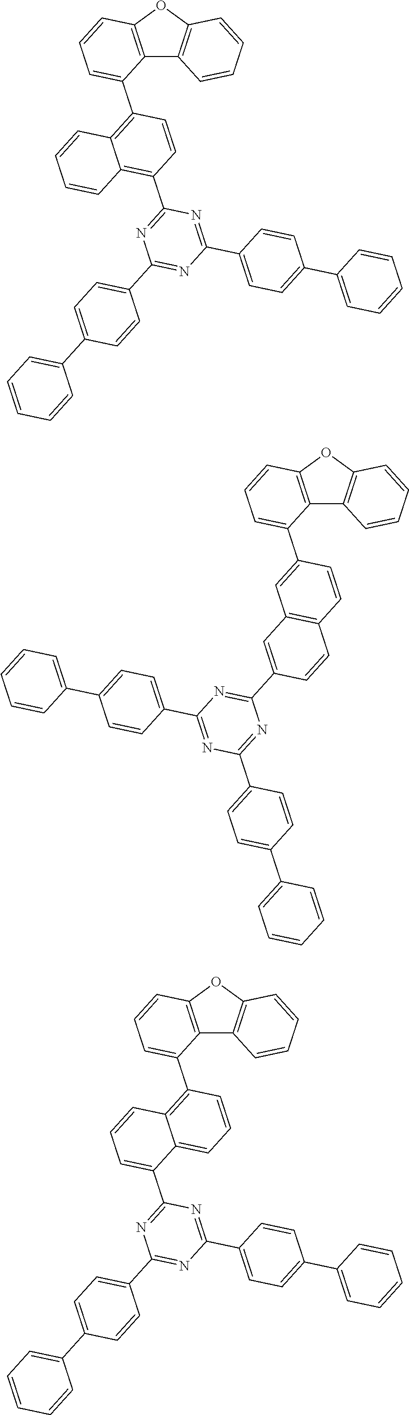 [SMILES: c1ccc(-c2ccc(-c3nc(-c4ccc(-c5ccccc5)cc4)nc(-c4ccc(-c5cccc6oc7ccccc7c56)c5ccccc45)n3)cc2)cc1 ; c1ccc(-c2ccc(-c3nc(-c4ccc(-c5ccccc5)cc4)nc(-c4ccc5ccc(-c6cccc7oc8ccccc8c67)cc5c4)n3)cc2)cc1 ; c1ccc(-c2ccc(-c3nc(-c4ccc(-c5ccccc5)cc4)nc(-c4cccc5c(-c6cccc7oc8ccccc8c67)cccc45)n3)cc2)cc1]

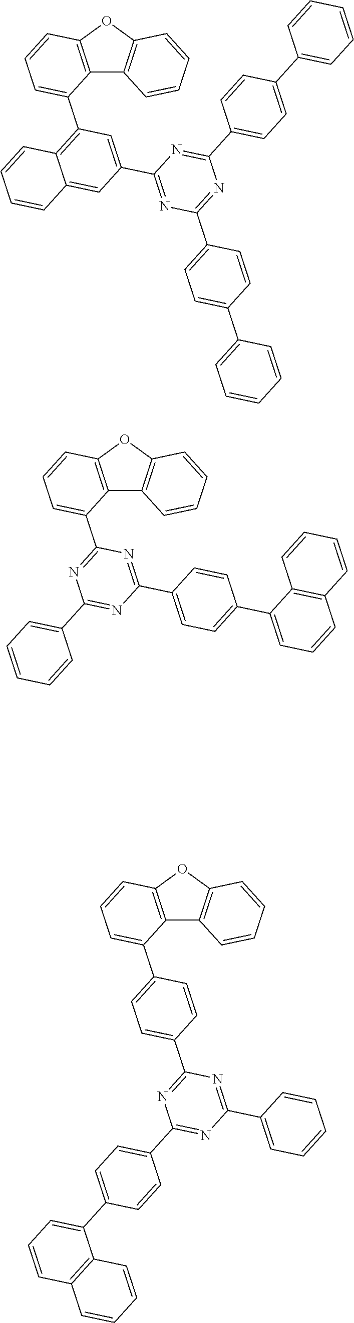 [SMILES: c1ccc(-c2ccc(-c3nc(-c4ccc(-c5ccccc5)cc4)nc(-c4cc(-c5cccc6oc7ccccc7c56)c5ccccc5c4)n3)cc2)cc1 ; c1ccc(-c2nc(-c3ccc(-c4cccc5ccccc45)cc3)nc(-c3cccc4oc5ccccc5c34)n2)cc1 ; c1ccc(-c2nc(-c3ccc(-c4cccc5ccccc45)cc3)nc(-c3ccc(-c4cccc5oc6ccccc6c45)cc3)n2)cc1]

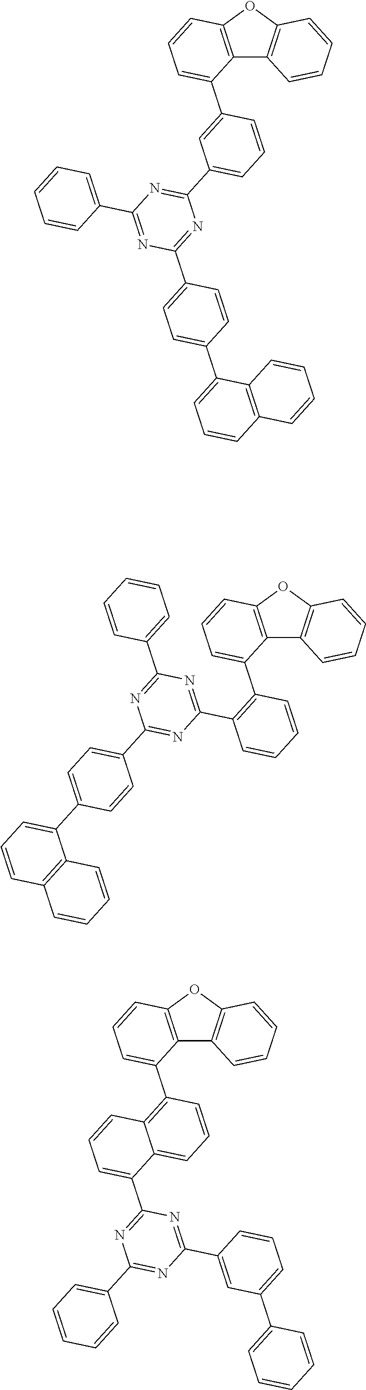 [SMILES: c1ccc(-c2nc(-c3ccc(-c4cccc5ccccc45)cc3)nc(-c3cccc(-c4cccc5oc6ccccc6c45)c3)n2)cc1 ; c1ccc(-c2nc(-c3ccc(-c4cccc5ccccc45)cc3)nc(-c3ccccc3-c3cccc4oc5ccccc5c34)n2)cc1 ; c1ccc(-c2cccc(-c3nc(-c4ccccc4)nc(-c4cccc5c(-c6cccc7oc8ccccc8c67)cccc45)n3)c2)cc1]

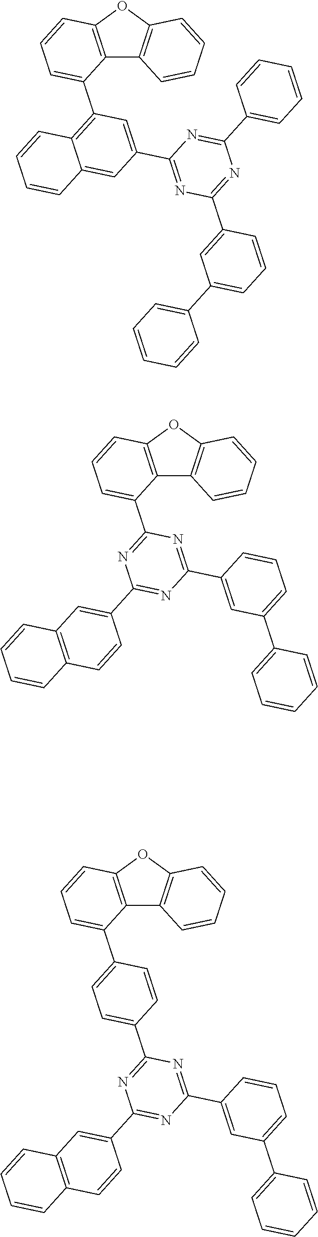 [SMILES: c1ccc(-c2cccc(-c3nc(-c4ccccc4)nc(-c4cc(-c5cccc6oc7ccccc7c56)c5ccccc5c4)n3)c2)cc1 ; c1ccc(-c2cccc(-c3nc(-c4ccc5ccccc5c4)nc(-c4cccc5oc6ccccc6c45)n3)c2)cc1 ; c1ccc(-c2cccc(-c3nc(-c4ccc(-c5cccc6oc7ccccc7c56)cc4)nc(-c4ccc5ccccc5c4)n3)c2)cc1]

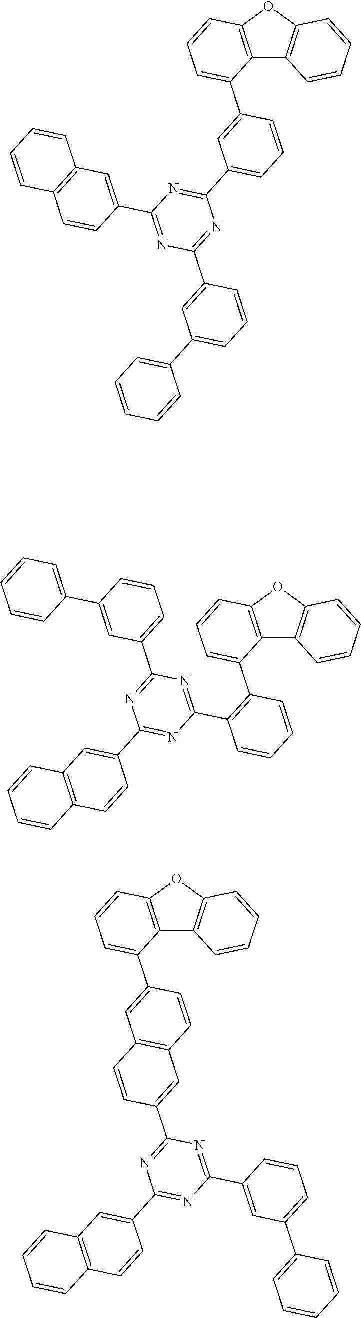 [SMILES: c1ccc(-c2cccc(-c3nc(-c4cccc(-c5cccc6oc7ccccc7c56)c4)nc(-c4ccc5ccccc5c4)n3)c2)cc1 ; c1ccc(-c2cccc(-c3nc(-c4ccc5ccccc5c4)nc(-c4ccccc4-c4cccc5oc6ccccc6c45)n3)c2)cc1 ; c1ccc(-c2cccc(-c3nc(-c4ccc5ccccc5c4)nc(-c4ccc5cc(-c6cccc7oc8ccccc8c67)ccc5c4)n3)c2)cc1]

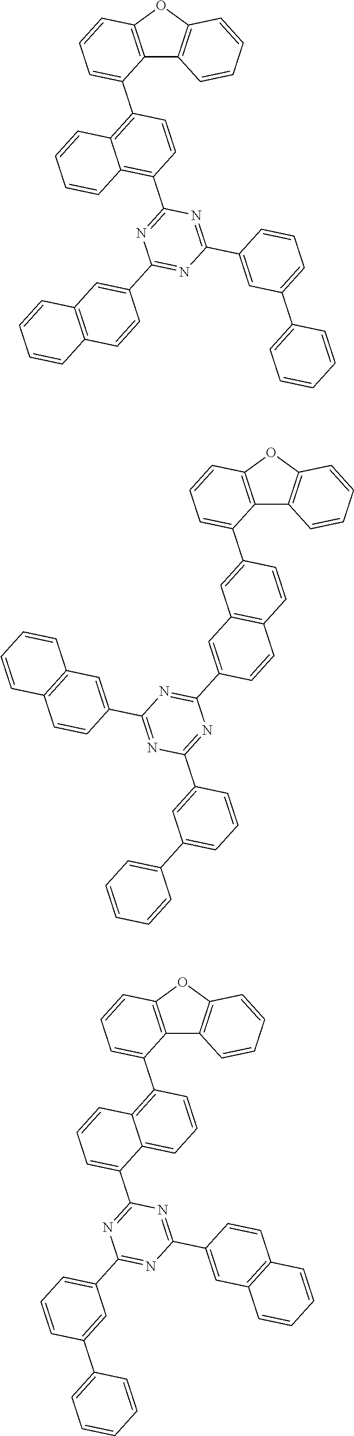 [SMILES: c1ccc(-c2cccc(-c3nc(-c4ccc5ccccc5c4)nc(-c4ccc(-c5cccc6oc7ccccc7c56)c5ccccc45)n3)c2)cc1 ; c1ccc(-c2cccc(-c3nc(-c4ccc5ccccc5c4)nc(-c4ccc5ccc(-c6cccc7oc8ccccc8c67)cc5c4)n3)c2)cc1 ; c1ccc(-c2cccc(-c3nc(-c4ccc5ccccc5c4)nc(-c4cccc5c(-c6cccc7oc8ccccc8c67)cccc45)n3)c2)cc1]

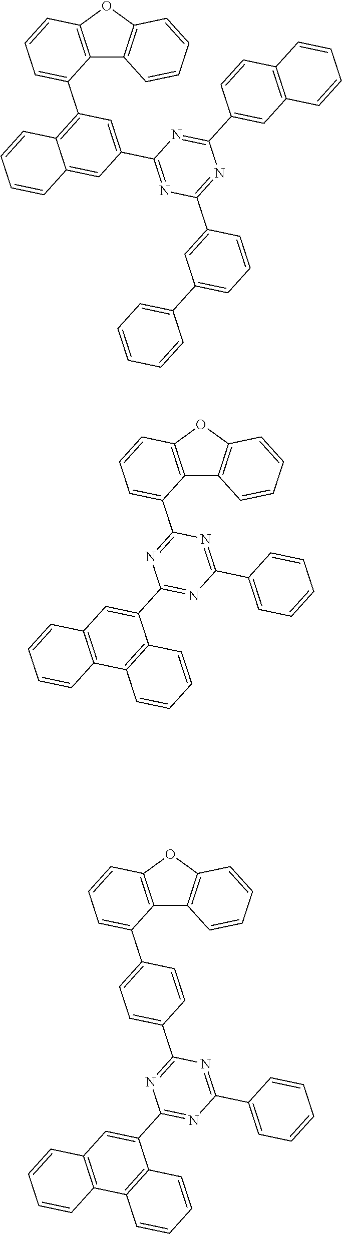 [SMILES: c1ccc(-c2cccc(-c3nc(-c4ccc5ccccc5c4)nc(-c4cc(-c5cccc6oc7ccccc7c56)c5ccccc5c4)n3)c2)cc1 ; c1ccc(-c2nc(-c3cc4ccccc4c4ccccc34)nc(-c3cccc4oc5ccccc5c34)n2)cc1 ; c1ccc(-c2nc(-c3ccc(-c4cccc5oc6ccccc6c45)cc3)nc(-c3cc4ccccc4c4ccccc34)n2)cc1]

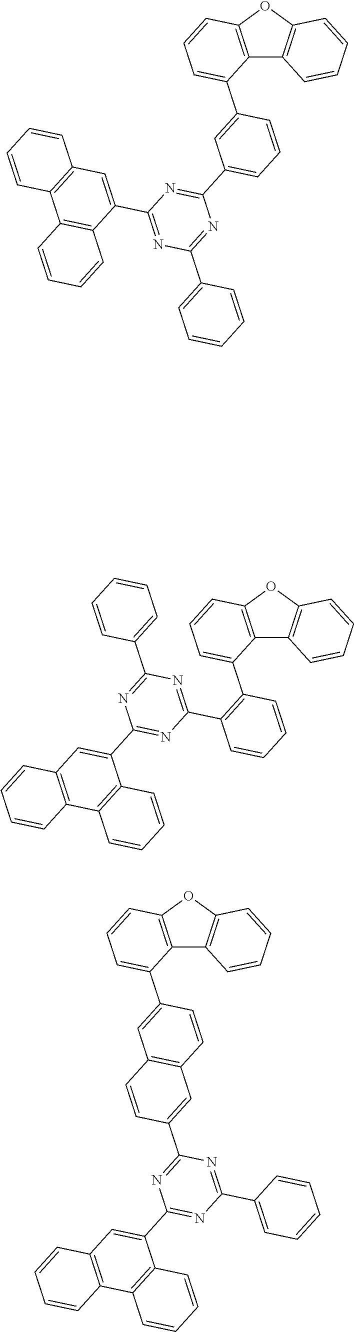 [SMILES: c1ccc(-c2nc(-c3cccc(-c4cccc5oc6ccccc6c45)c3)nc(-c3cc4ccccc4c4ccccc34)n2)cc1 ; c1ccc(-c2nc(-c3ccccc3-c3cccc4oc5ccccc5c34)nc(-c3cc4ccccc4c4ccccc34)n2)cc1 ; c1ccc(-c2nc(-c3ccc4cc(-c5cccc6oc7ccccc7c56)ccc4c3)nc(-c3cc4ccccc4c4ccccc34)n2)cc1]

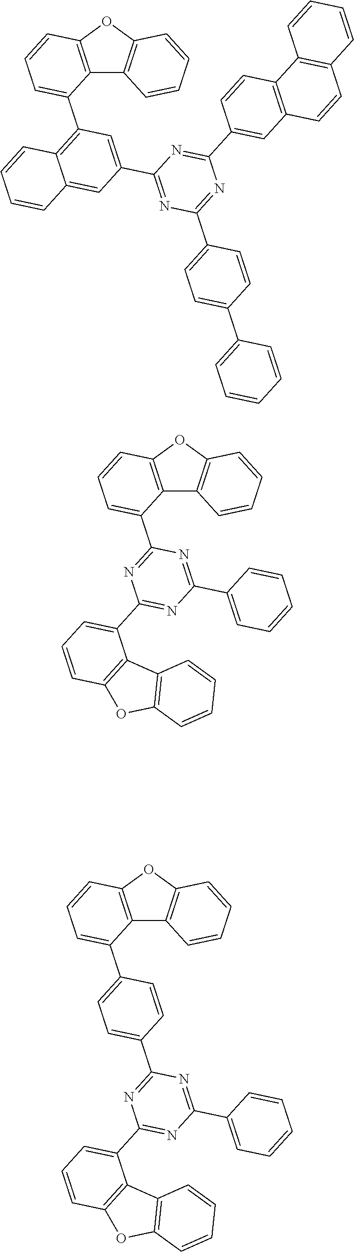 [SMILES: c1ccc(-c2ccc(-c3nc(-c4cc(-c5cccc6oc7ccccc7c56)c5ccccc5c4)nc(-c4ccc5c(ccc6ccccc65)c4)n3)cc2)cc1 ; c1ccc(-c2nc(-c3cccc4oc5ccccc5c34)nc(-c3cccc4oc5ccccc5c34)n2)cc1 ; c1ccc(-c2nc(-c3ccc(-c4cccc5oc6ccccc6c45)cc3)nc(-c3cccc4oc5ccccc5c34)n2)cc1]

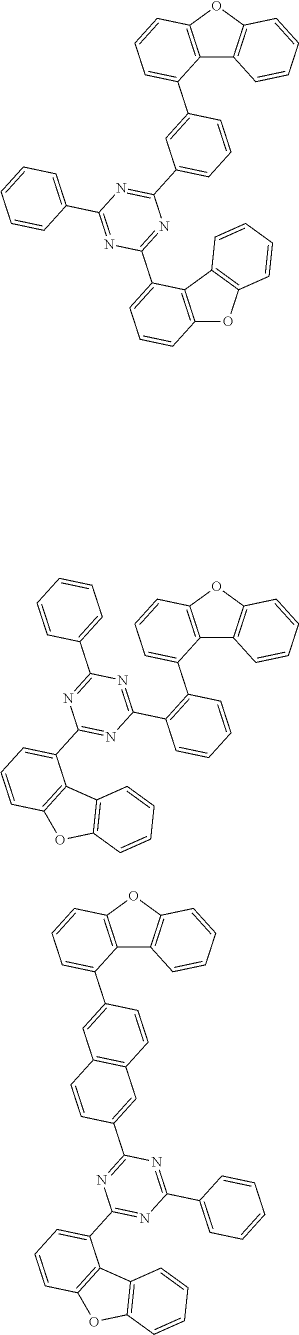

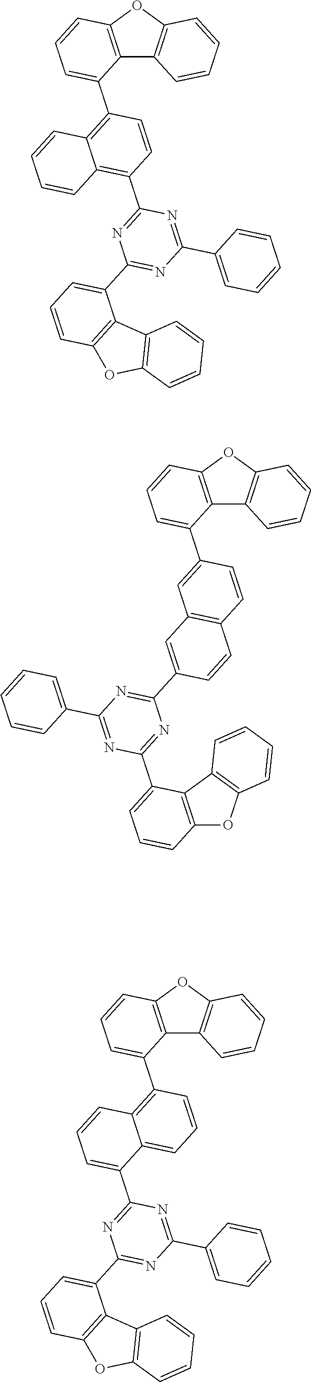 [SMILES: c1ccc(-c2nc(-c3ccc(-c4cccc5oc6ccccc6c45)c4ccccc34)nc(-c3cccc4oc5ccccc5c34)n2)cc1 ; c1ccc(-c2nc(-c3ccc4ccc(-c5cccc6oc7ccccc7c56)cc4c3)nc(-c3cccc4oc5ccccc5c34)n2)cc1 ; c1ccc(-c2nc(-c3cccc4c(-c5cccc6oc7ccccc7c56)cccc34)nc(-c3cccc4oc5ccccc5c34)n2)cc1]

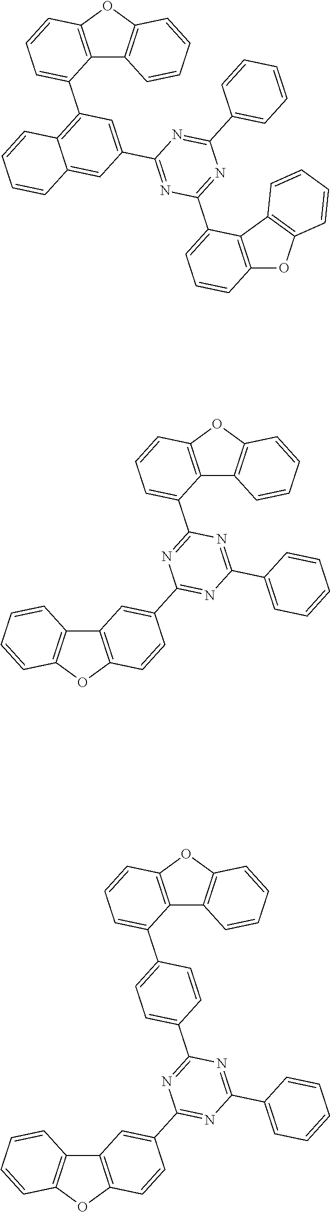 [SMILES: c1ccc(-c2nc(-c3cc(-c4cccc5oc6ccccc6c45)c4ccccc4c3)nc(-c3cccc4oc5ccccc5c34)n2)cc1 ; c1ccc(-c2nc(-c3ccc4oc5ccccc5c4c3)nc(-c3cccc4oc5ccccc5c34)n2)cc1 ; c1ccc(-c2nc(-c3ccc(-c4cccc5oc6ccccc6c45)cc3)nc(-c3ccc4oc5ccccc5c4c3)n2)cc1]

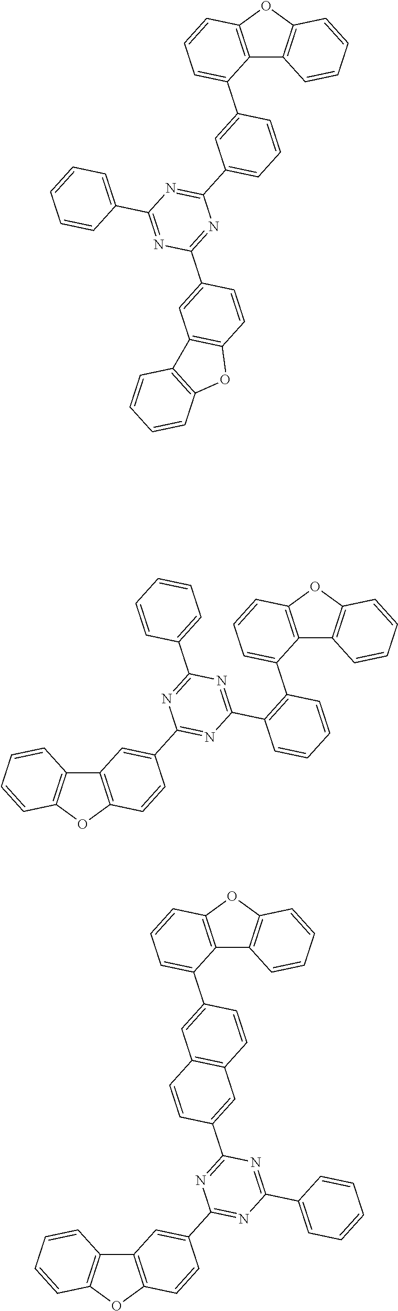 [SMILES: c1ccc(-c2nc(-c3cccc(-c4cccc5oc6ccccc6c45)c3)nc(-c3ccc4oc5ccccc5c4c3)n2)cc1 ; c1ccc(-c2nc(-c3ccc4oc5ccccc5c4c3)nc(-c3ccccc3-c3cccc4oc5ccccc5c34)n2)cc1 ; c1ccc(-c2nc(-c3ccc4cc(-c5cccc6oc7ccccc7c56)ccc4c3)nc(-c3ccc4oc5ccccc5c4c3)n2)cc1]

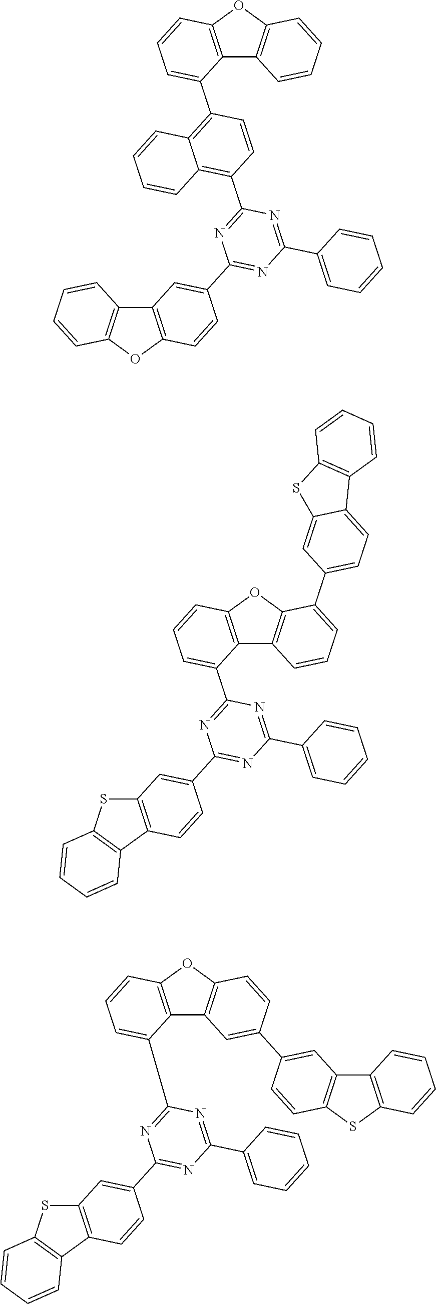 [SMILES: c1ccc(-c2nc(-c3ccc4oc5ccccc5c4c3)nc(-c3ccc(-c4cccc5oc6ccccc6c45)c4ccccc34)n2)cc1 ; c1ccc(-c2nc(-c3ccc4c(c3)sc3ccccc34)nc(-c3cccc4oc5c(-c6ccc7c(c6)sc6ccccc67)cccc5c34)n2)cc1 ; c1ccc(-c2nc(-c3ccc4c(c3)sc3ccccc34)nc(-c3cccc4oc5ccc(-c6ccc7sc8ccccc8c7c6)cc5c34)n2)cc1]

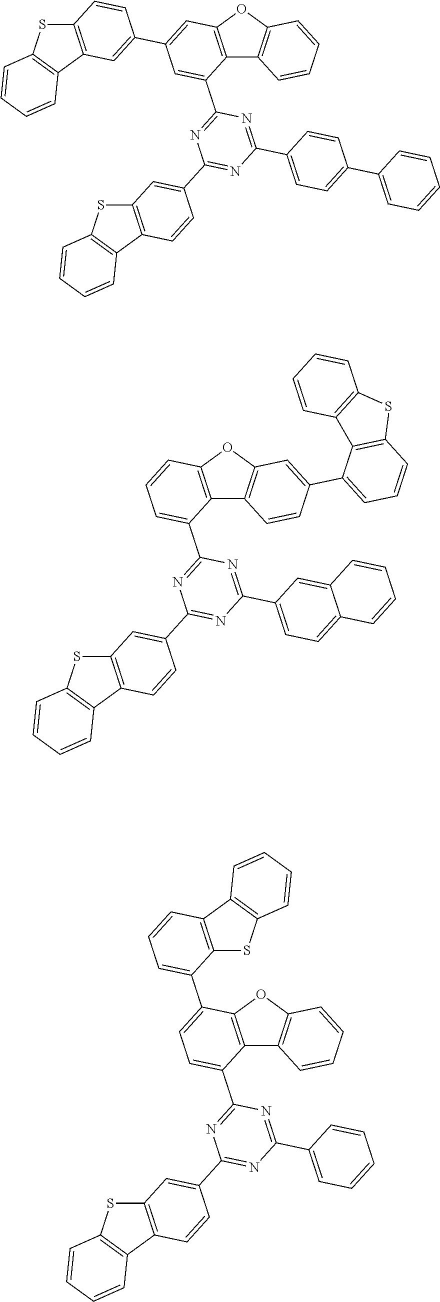 [SMILES: c1ccc(-c2ccc(-c3nc(-c4ccc5c(c4)sc4ccccc45)nc(-c4cc(-c5ccc6sc7ccccc7c6c5)cc5oc6ccccc6c45)n3)cc2)cc1 ; c1ccc2cc(-c3nc(-c4ccc5c(c4)sc4ccccc45)nc(-c4cccc5oc6cc(-c7cccc8sc9ccccc9c78)ccc6c45)n3)ccc2c1 ; c1ccc(-c2nc(-c3ccc4c(c3)sc3ccccc34)nc(-c3ccc(-c4cccc5c4sc4ccccc45)c4oc5ccccc5c34)n2)cc1]

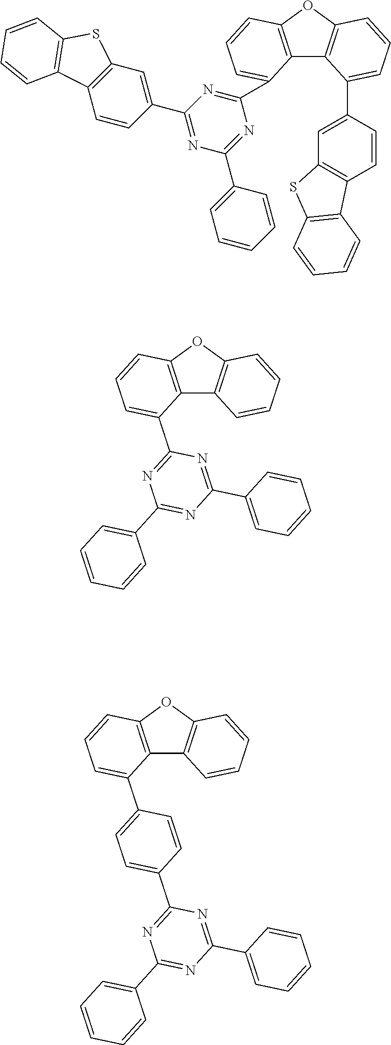 [SMILES: c1ccc(-c2nc(-c3ccc4c(c3)sc3ccccc34)nc(-c3cccc4oc5cccc(-c6ccc7c(c6)sc6ccccc67)c5c34)n2)cc1 ; c1ccc(-c2nc(-c3ccccc3)nc(-c3cccc4oc5ccccc5c34)n2)cc1 ; c1ccc(-c2nc(-c3ccccc3)nc(-c3ccc(-c4cccc5oc6ccccc6c45)cc3)n2)cc1]

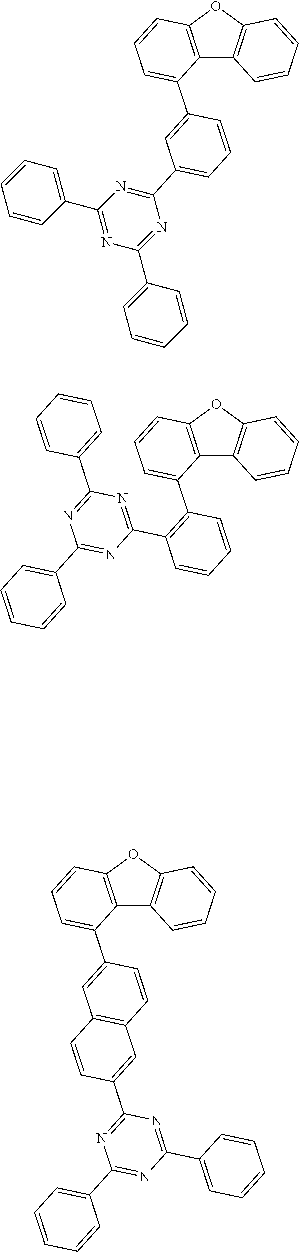 [SMILES: c1ccc(-c2nc(-c3ccccc3)nc(-c3cccc(-c4cccc5oc6ccccc6c45)c3)n2)cc1 ; c1ccc(-c2nc(-c3ccccc3)nc(-c3ccccc3-c3cccc4oc5ccccc5c34)n2)cc1 ; c1ccc(-c2nc(-c3ccccc3)nc(-c3ccc4cc(-c5cccc6oc7ccccc7c56)ccc4c3)n2)cc1]

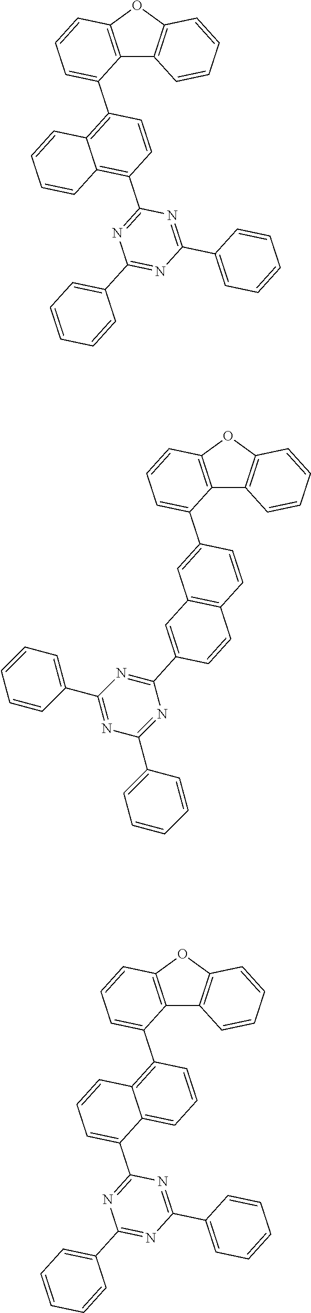 [SMILES: c1ccc(-c2nc(-c3ccccc3)nc(-c3ccc(-c4cccc5oc6ccccc6c45)c4ccccc34)n2)cc1 ; c1ccc(-c2nc(-c3ccccc3)nc(-c3ccc4ccc(-c5cccc6oc7ccccc7c56)cc4c3)n2)cc1 ; c1ccc(-c2nc(-c3ccccc3)nc(-c3cccc4c(-c5cccc6oc7ccccc7c56)cccc34)n2)cc1]

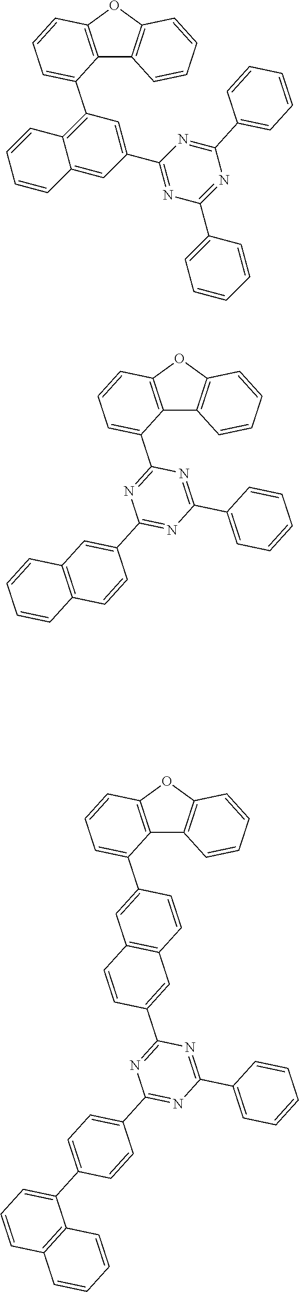 [SMILES: c1ccc(-c2nc(-c3ccccc3)nc(-c3cc(-c4cccc5oc6ccccc6c45)c4ccccc4c3)n2)cc1 ; c1ccc(-c2nc(-c3ccc4ccccc4c3)nc(-c3cccc4oc5ccccc5c34)n2)cc1 ; c1ccc(-c2nc(-c3ccc(-c4cccc5ccccc45)cc3)nc(-c3ccc4cc(-c5cccc6oc7ccccc7c56)ccc4c3)n2)cc1]

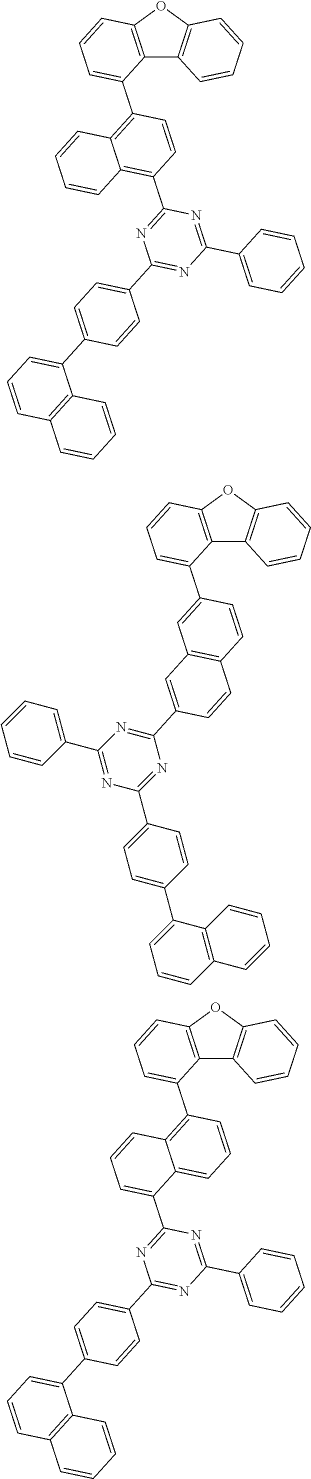

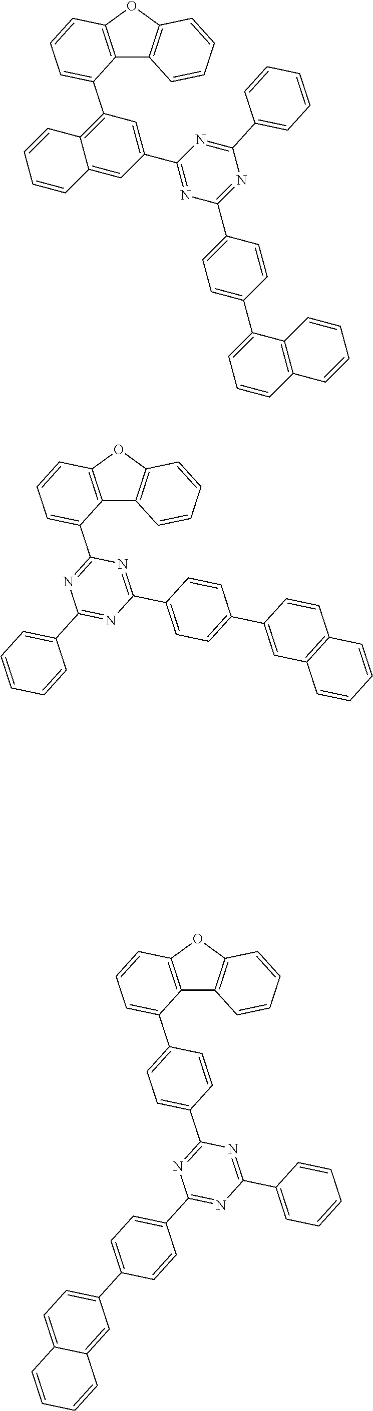 [SMILES: c1ccc(-c2nc(-c3ccc(-c4cccc5ccccc45)cc3)nc(-c3cc(-c4cccc5oc6ccccc6c45)c4ccccc4c3)n2)cc1 ; c1ccc(-c2nc(-c3ccc(-c4ccc5ccccc5c4)cc3)nc(-c3cccc4oc5ccccc5c34)n2)cc1 ; c1ccc(-c2nc(-c3ccc(-c4ccc5ccccc5c4)cc3)nc(-c3ccc(-c4cccc5oc6ccccc6c45)cc3)n2)cc1]

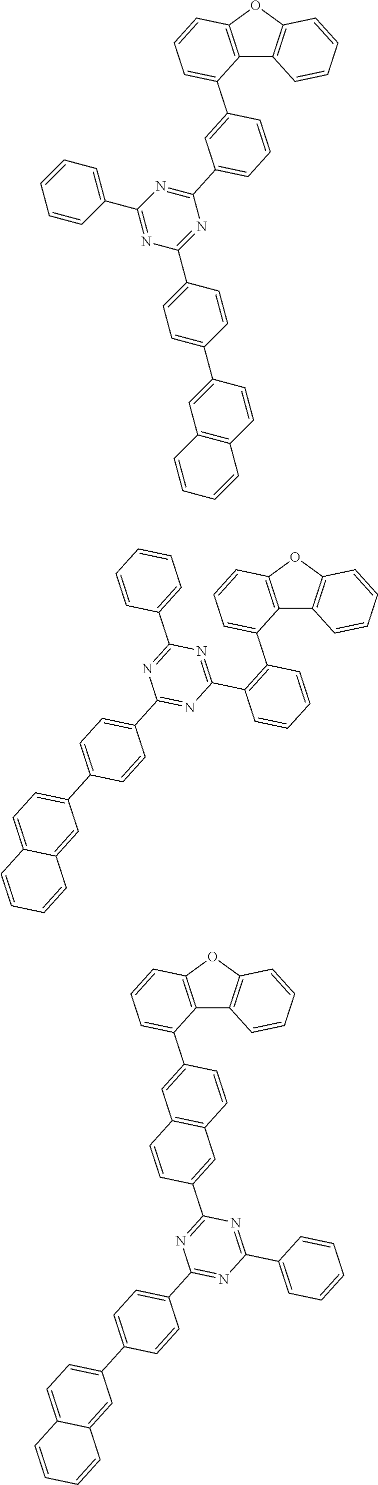 [SMILES: c1ccc(-c2nc(-c3ccc(-c4ccc5ccccc5c4)cc3)nc(-c3cccc(-c4cccc5oc6ccccc6c45)c3)n2)cc1 ; c1ccc(-c2nc(-c3ccc(-c4ccc5ccccc5c4)cc3)nc(-c3ccccc3-c3cccc4oc5ccccc5c34)n2)cc1 ; c1ccc(-c2nc(-c3ccc(-c4ccc5ccccc5c4)cc3)nc(-c3ccc4cc(-c5cccc6oc7ccccc7c56)ccc4c3)n2)cc1]

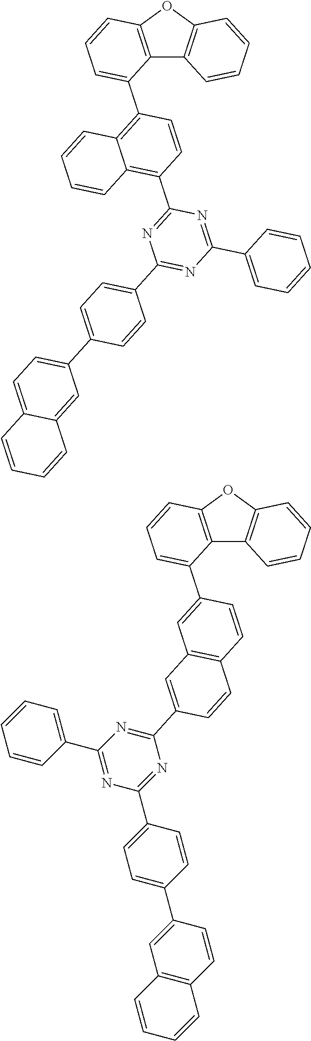 [SMILES: c1ccc(-c2nc(-c3ccc(-c4ccc5ccccc5c4)cc3)nc(-c3ccc(-c4cccc5oc6ccccc6c45)c4ccccc34)n2)cc1 ; c1ccc(-c2nc(-c3ccc(-c4ccc5ccccc5c4)cc3)nc(-c3ccc4ccc(-c5cccc6oc7ccccc7c56)cc4c3)n2)cc1]

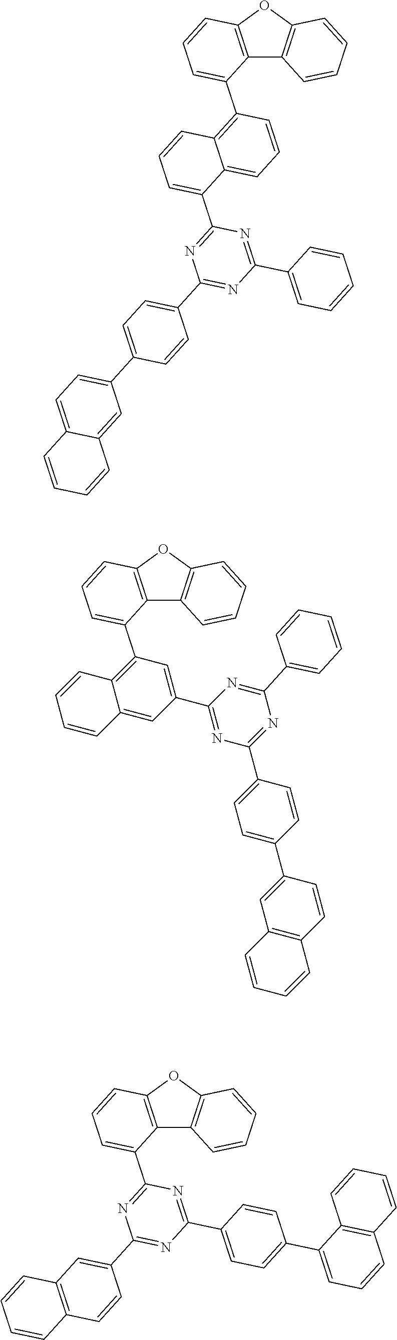 [SMILES: c1ccc(-c2nc(-c3ccc(-c4ccc5ccccc5c4)cc3)nc(-c3cccc4c(-c5cccc6oc7ccccc7c56)cccc34)n2)cc1 ; c1ccc(-c2nc(-c3ccc(-c4ccc5ccccc5c4)cc3)nc(-c3cc(-c4cccc5oc6ccccc6c45)c4ccccc4c3)n2)cc1 ; c1ccc2cc(-c3nc(-c4ccc(-c5cccc6ccccc56)cc4)nc(-c4cccc5oc6ccccc6c45)n3)ccc2c1]

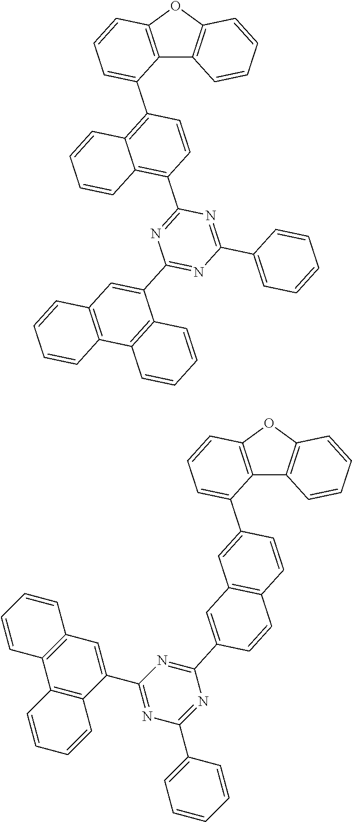 [SMILES: c1ccc(-c2nc(-c3ccc(-c4cccc5oc6ccccc6c45)c4ccccc34)nc(-c3cc4ccccc4c4ccccc34)n2)cc1 ; c1ccc(-c2nc(-c3ccc4ccc(-c5cccc6oc7ccccc7c56)cc4c3)nc(-c3cc4ccccc4c4ccccc34)n2)cc1]

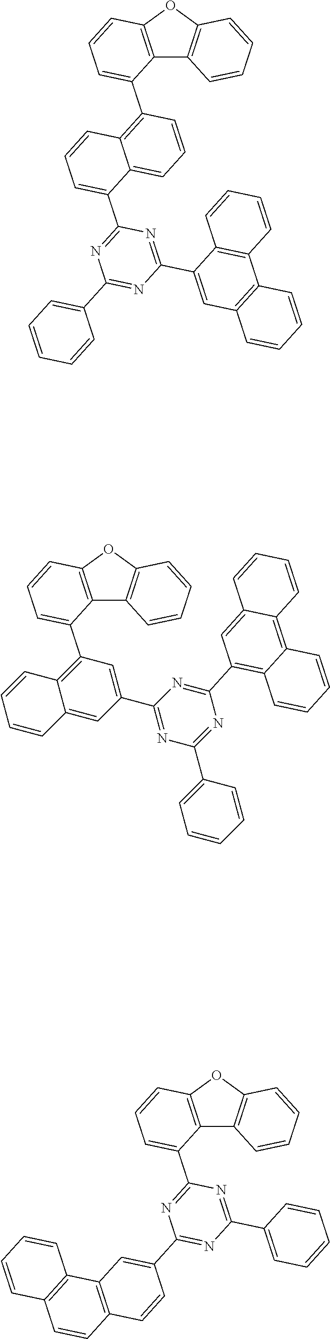 [SMILES: c1ccc(-c2nc(-c3cccc4c(-c5cccc6oc7ccccc7c56)cccc34)nc(-c3cc4ccccc4c4ccccc34)n2)cc1 ; c1ccc(-c2nc(-c3cc(-c4cccc5oc6ccccc6c45)c4ccccc4c3)nc(-c3cc4ccccc4c4ccccc34)n2)cc1 ; c1ccc(-c2nc(-c3ccc4ccc5ccccc5c4c3)nc(-c3cccc4oc5ccccc5c34)n2)cc1]

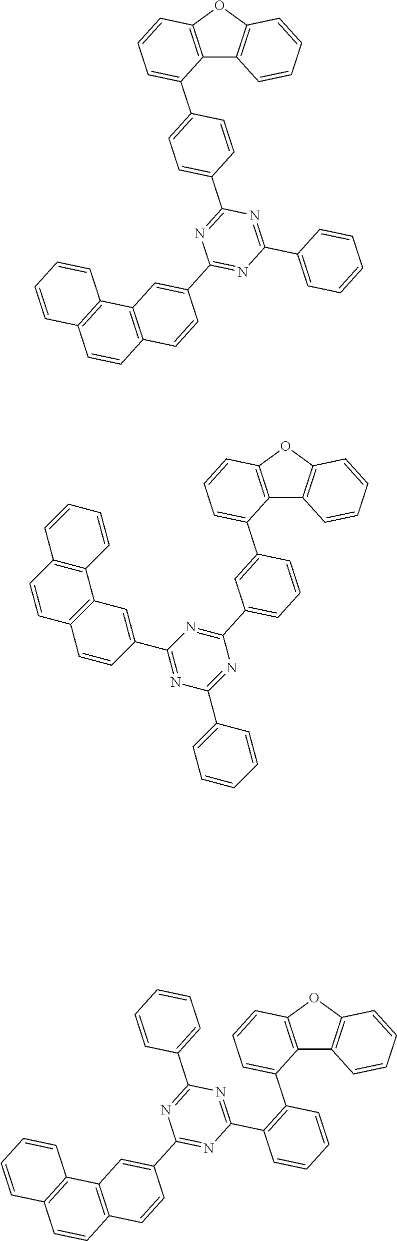 [SMILES: c1ccc(-c2nc(-c3ccc(-c4cccc5oc6ccccc6c45)cc3)nc(-c3ccc4ccc5ccccc5c4c3)n2)cc1 ; c1ccc(-c2nc(-c3cccc(-c4cccc5oc6ccccc6c45)c3)nc(-c3ccc4ccc5ccccc5c4c3)n2)cc1 ; c1ccc(-c2nc(-c3ccc4ccc5ccccc5c4c3)nc(-c3ccccc3-c3cccc4oc5ccccc5c34)n2)cc1]

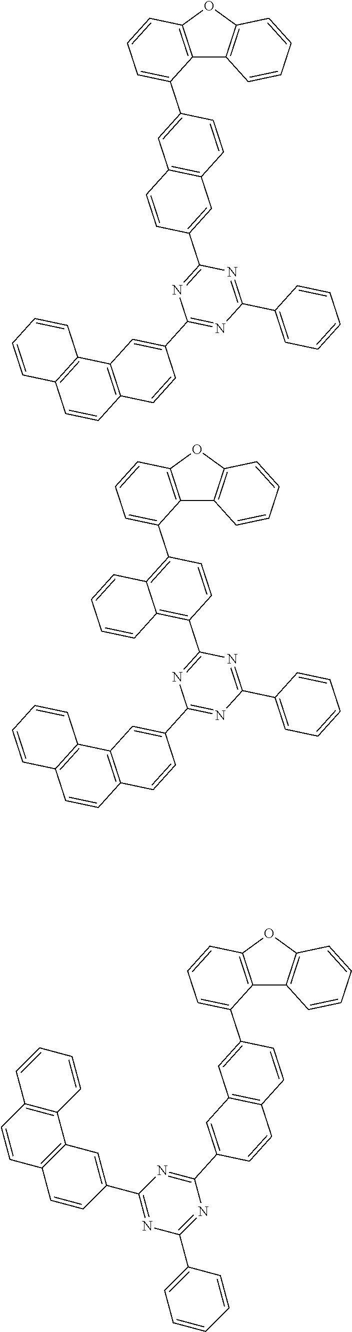 [SMILES: c1ccc(-c2nc(-c3ccc4cc(-c5cccc6oc7ccccc7c56)ccc4c3)nc(-c3ccc4ccc5ccccc5c4c3)n2)cc1 ; c1ccc(-c2nc(-c3ccc4ccc5ccccc5c4c3)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4ccc5ccccc5c4c3)n2)cc1]

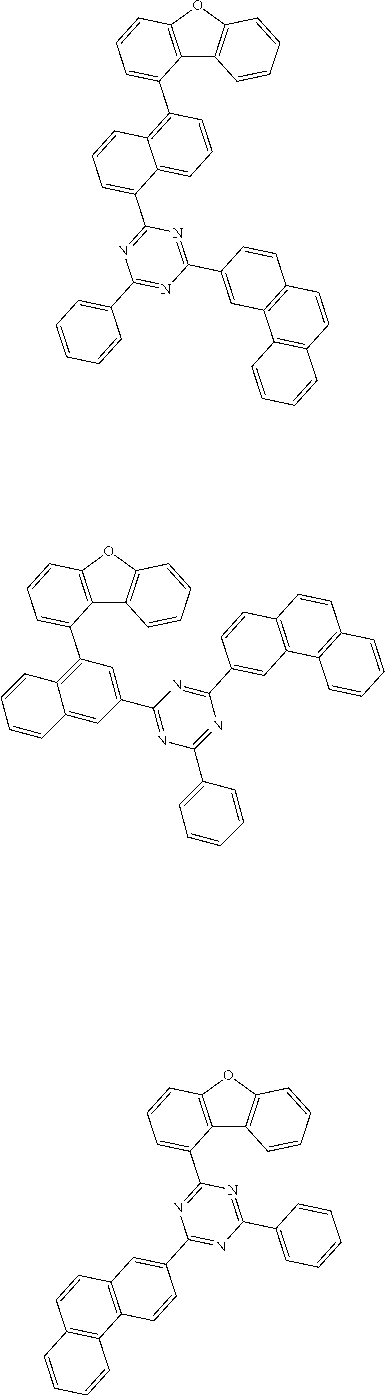 [SMILES: c1ccc(-c2nc(-c3ccc4ccc5ccccc5c4c3)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4ccc5ccccc5c4c3)n2)cc1 ; c1ccc(-c2nc(-c3ccc4c(ccc5ccccc54)c3)nc(-c3cccc4oc5ccccc5c34)n2)cc1]

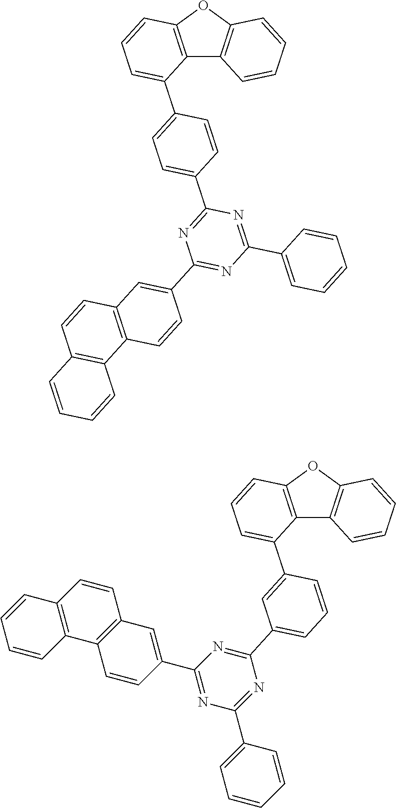 [SMILES: c1ccc(-c2nc(-c3ccc(-c4cccc5oc6ccccc6c45)cc3)nc(-c3ccc4c(ccc5ccccc54)c3)n2)cc1 ; c1ccc(-c2nc(-c3cccc(-c4cccc5oc6ccccc6c45)c3)nc(-c3ccc4c(ccc5ccccc54)c3)n2)cc1]

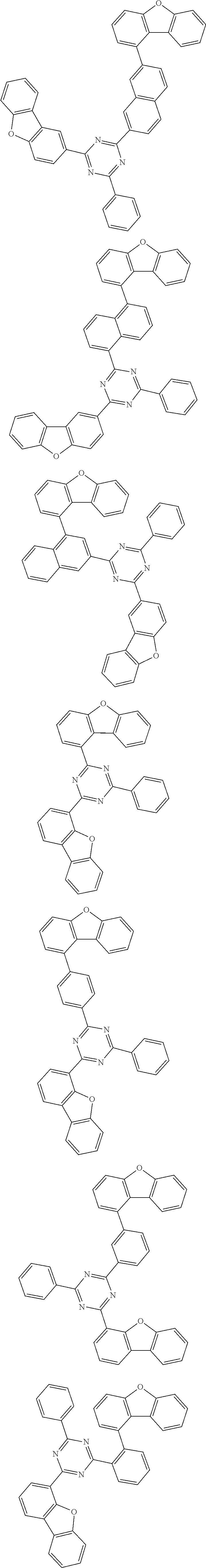

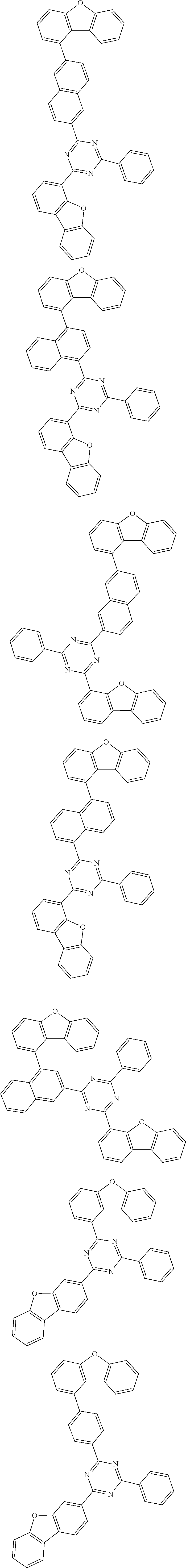

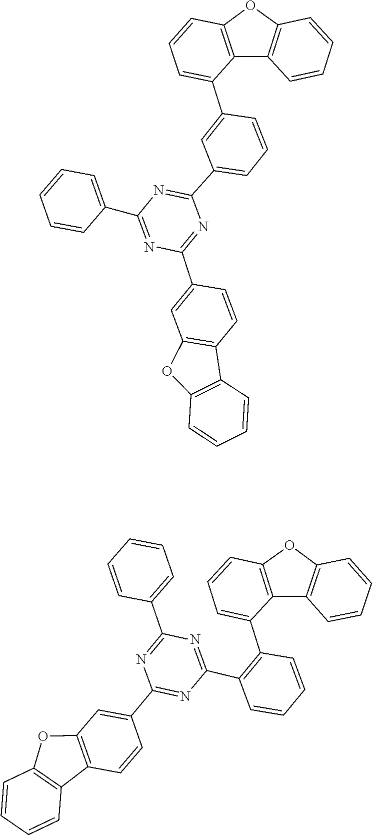 [SMILES: c1ccc(-c2nc(-c3cccc(-c4cccc5oc6ccccc6c45)c3)nc(-c3ccc4c(c3)oc3ccccc34)n2)cc1 ; c1ccc(-c2nc(-c3ccc4c(c3)oc3ccccc34)nc(-c3ccccc3-c3cccc4oc5ccccc5c34)n2)cc1]

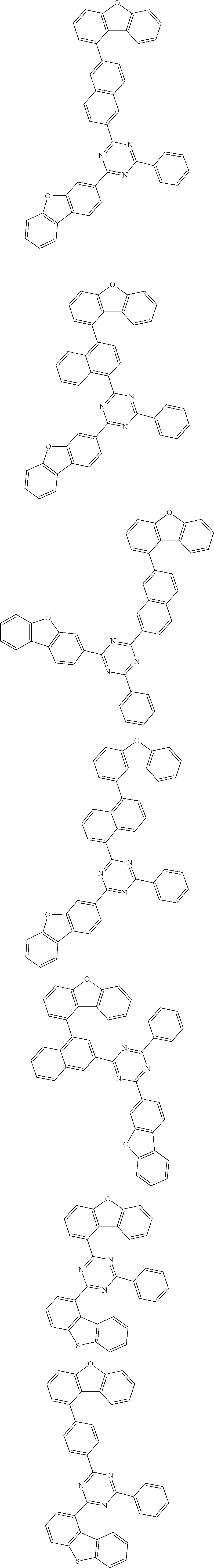

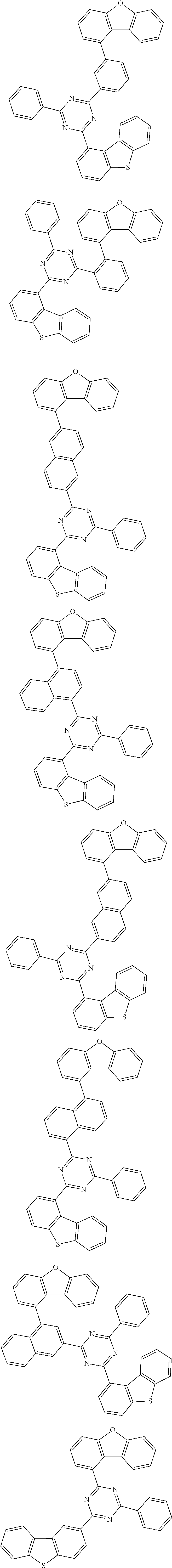

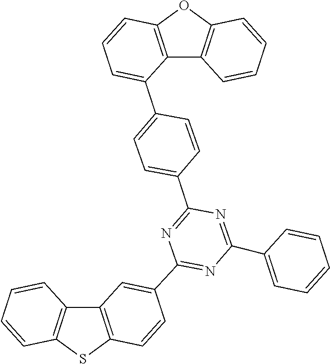 [SMILES: c1ccc(-c2nc(-c3ccc(-c4cccc5oc6ccccc6c45)cc3)nc(-c3ccc4sc5ccccc5c4c3)n2)cc1]

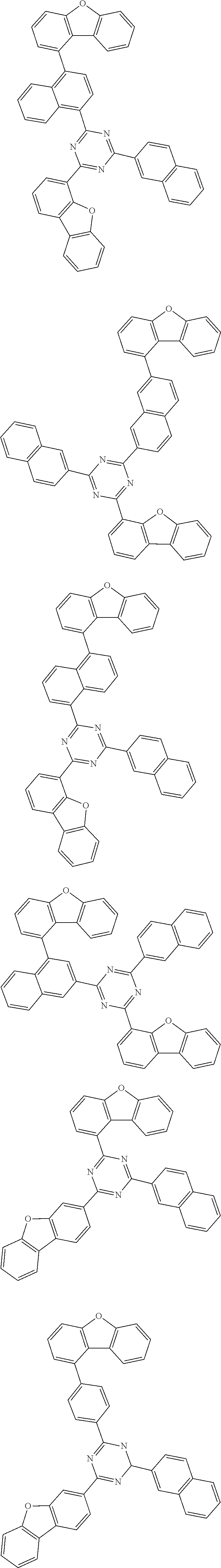

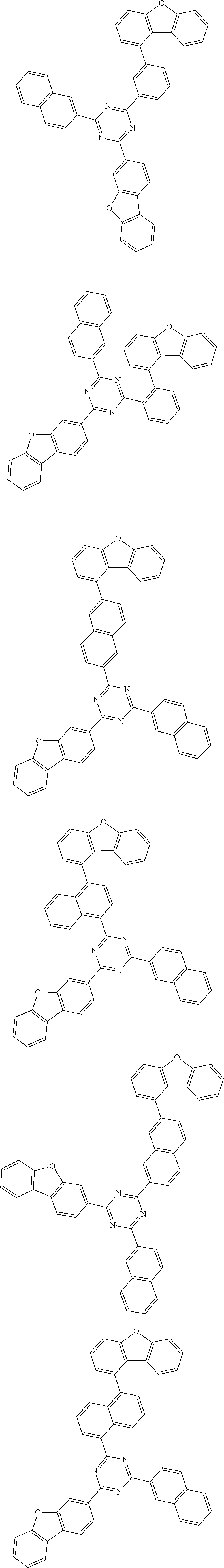

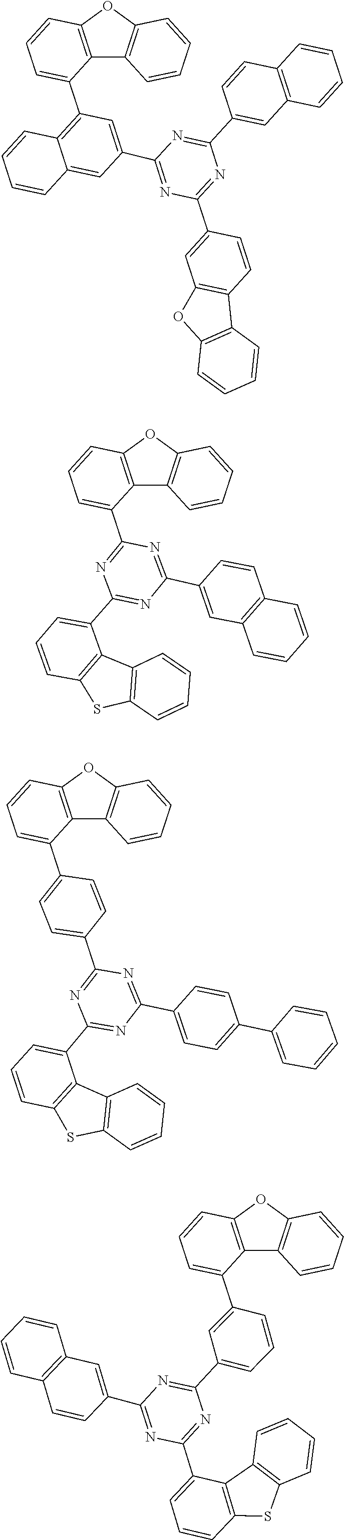 [SMILES: c1ccc2cc(-c3nc(-c4cc(-c5cccc6oc7ccccc7c56)c5ccccc5c4)nc(-c4ccc5c(c4)oc4ccccc45)n3)ccc2c1 ; c1ccc2cc(-c3nc(-c4cccc5oc6ccccc6c45)nc(-c4cccc5sc6ccccc6c45)n3)ccc2c1 ; c1ccc(-c2ccc(-c3nc(-c4ccc(-c5cccc6oc7ccccc7c56)cc4)nc(-c4cccc5sc6ccccc6c45)n3)cc2)cc1 ; c1cc(-c2nc(-c3ccc4ccccc4c3)nc(-c3cccc4sc5ccccc5c34)n2)cc(-c2cccc3oc4ccccc4c23)c1]

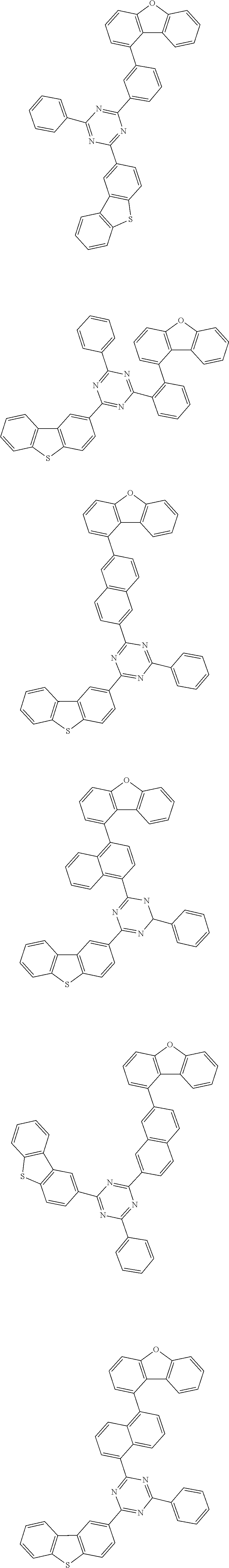

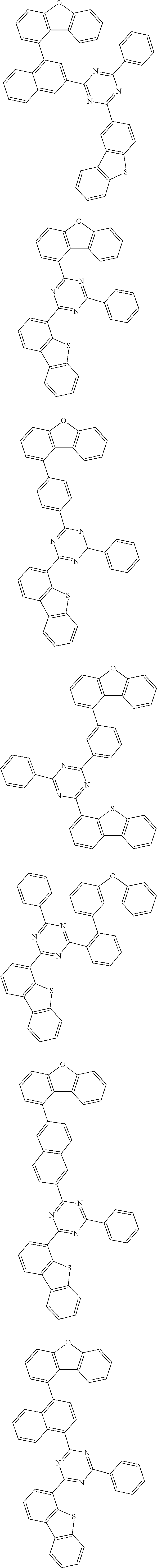

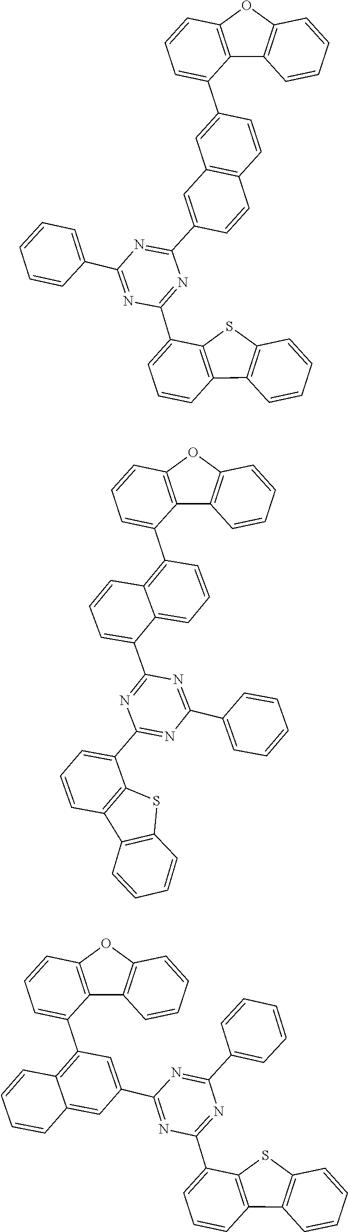 [SMILES: c1ccc(-c2nc(-c3ccc4ccc(-c5cccc6oc7ccccc7c56)cc4c3)nc(-c3cccc4c3sc3ccccc34)n2)cc1 ; c1ccc(-c2nc(-c3cccc4c(-c5cccc6oc7ccccc7c56)cccc34)nc(-c3cccc4c3sc3ccccc34)n2)cc1 ; c1ccc(-c2nc(-c3cc(-c4cccc5oc6ccccc6c45)c4ccccc4c3)nc(-c3cccc4c3sc3ccccc34)n2)cc1]

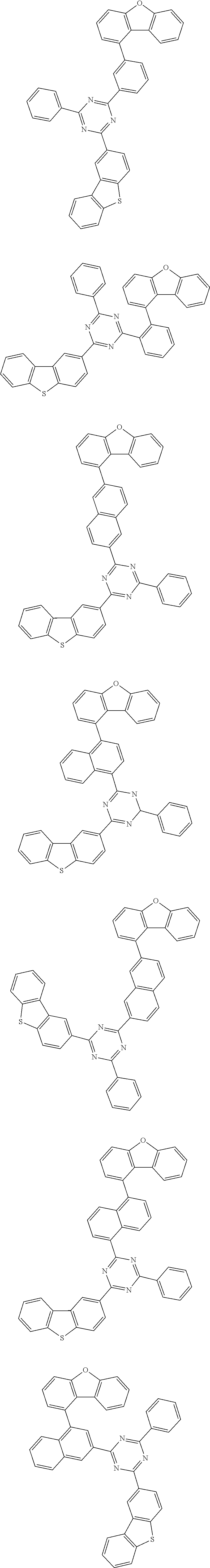

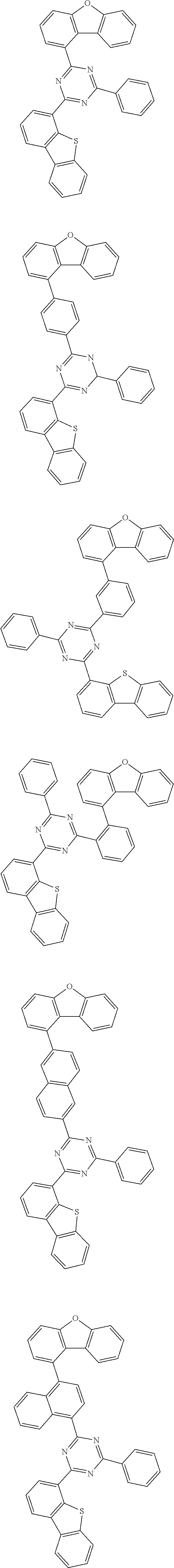

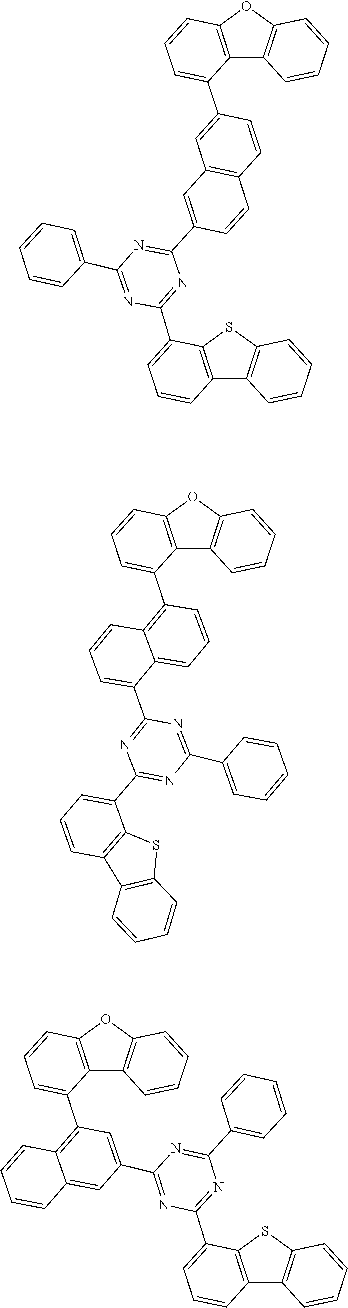 [SMILES: c1ccc(-c2nc(-c3ccc4ccc(-c5cccc6oc7ccccc7c56)cc4c3)nc(-c3cccc4c3sc3ccccc34)n2)cc1 ; c1ccc(-c2nc(-c3cccc4c(-c5cccc6oc7ccccc7c56)cccc34)nc(-c3cccc4c3sc3ccccc34)n2)cc1 ; c1ccc(-c2nc(-c3cc(-c4cccc5oc6ccccc6c45)c4ccccc4c3)nc(-c3cccc4c3sc3ccccc34)n2)cc1]

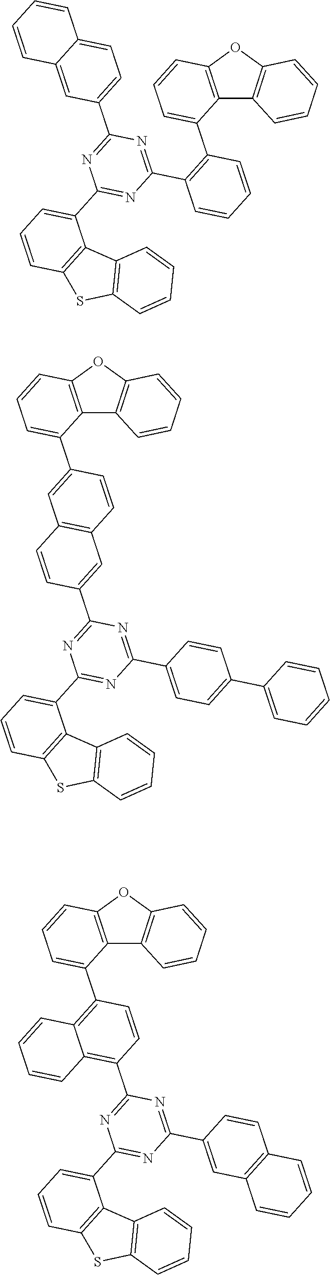 [SMILES: c1ccc(-c2cccc3oc4ccccc4c23)c(-c2nc(-c3ccc4ccccc4c3)nc(-c3cccc4sc5ccccc5c34)n2)c1 ; c1ccc(-c2ccc(-c3nc(-c4ccc5cc(-c6cccc7oc8ccccc8c67)ccc5c4)nc(-c4cccc5sc6ccccc6c45)n3)cc2)cc1 ; c1ccc2cc(-c3nc(-c4ccc(-c5cccc6oc7ccccc7c56)c5ccccc45)nc(-c4cccc5sc6ccccc6c45)n3)ccc2c1]

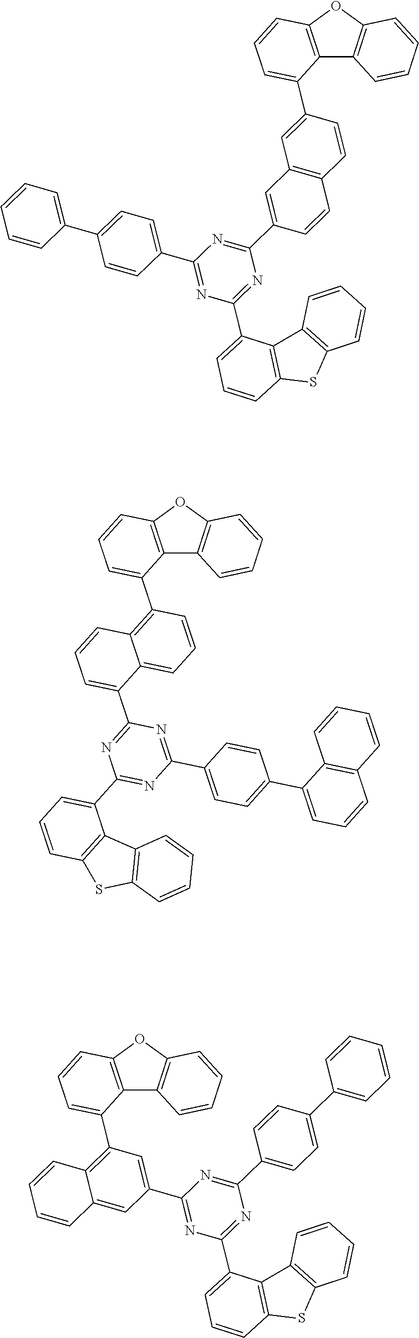 [SMILES: c1ccc(-c2ccc(-c3nc(-c4ccc5ccc(-c6cccc7oc8ccccc8c67)cc5c4)nc(-c4cccc5sc6ccccc6c45)n3)cc2)cc1 ; c1ccc2c(-c3ccc(-c4nc(-c5cccc6c(-c7cccc8oc9ccccc9c78)cccc56)nc(-c5cccc6sc7ccccc7c56)n4)cc3)cccc2c1 ; c1ccc(-c2ccc(-c3nc(-c4cc(-c5cccc6oc7ccccc7c56)c5ccccc5c4)nc(-c4cccc5sc6ccccc6c45)n3)cc2)cc1]

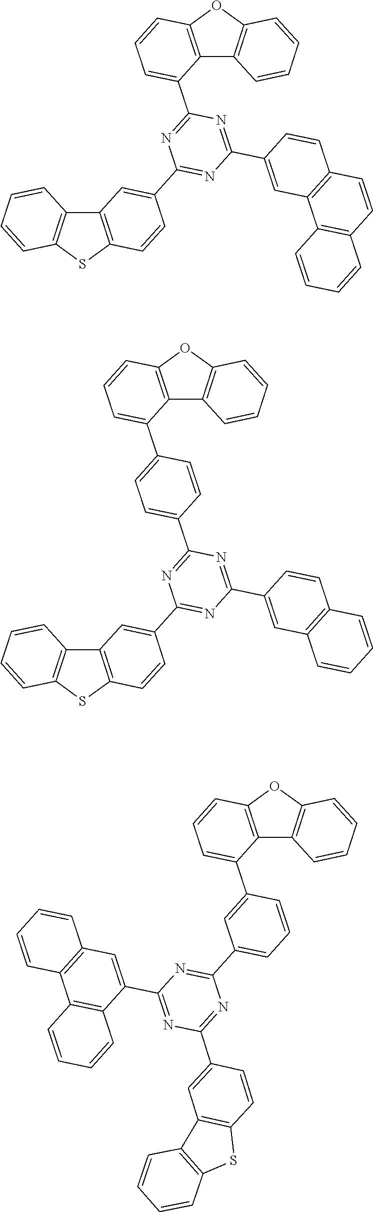 [SMILES: c1ccc2c(c1)ccc1ccc(-c3nc(-c4ccc5sc6ccccc6c5c4)nc(-c4cccc5oc6ccccc6c45)n3)cc12 ; c1ccc2cc(-c3nc(-c4ccc(-c5cccc6oc7ccccc7c56)cc4)nc(-c4ccc5sc6ccccc6c5c4)n3)ccc2c1 ; c1cc(-c2nc(-c3ccc4sc5ccccc5c4c3)nc(-c3cc4ccccc4c4ccccc34)n2)cc(-c2cccc3oc4ccccc4c23)c1]

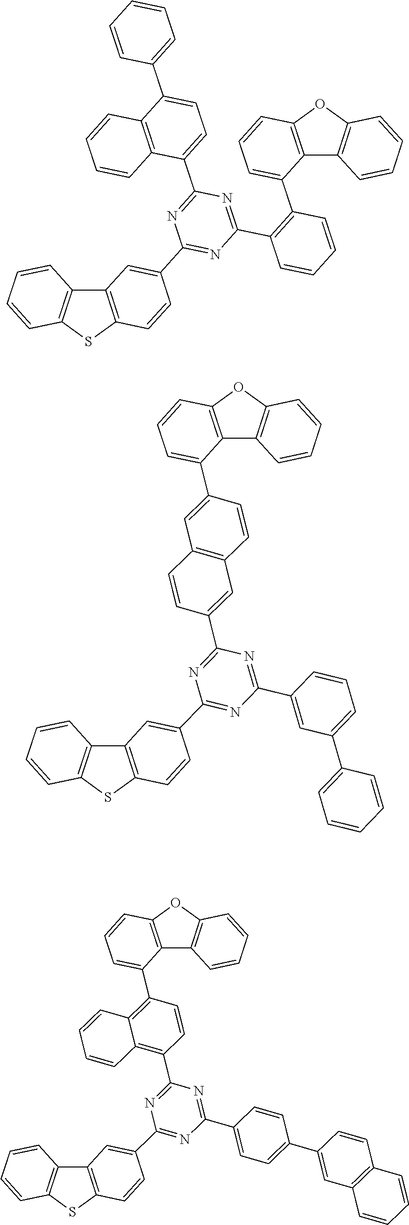 [SMILES: c1ccc(-c2ccc(-c3nc(-c4ccc5sc6ccccc6c5c4)nc(-c4ccccc4-c4cccc5oc6ccccc6c45)n3)c3ccccc23)cc1 ; c1ccc(-c2cccc(-c3nc(-c4ccc5cc(-c6cccc7oc8ccccc8c67)ccc5c4)nc(-c4ccc5sc6ccccc6c5c4)n3)c2)cc1 ; c1ccc2cc(-c3ccc(-c4nc(-c5ccc6sc7ccccc7c6c5)nc(-c5ccc(-c6cccc7oc8ccccc8c67)c6ccccc56)n4)cc3)ccc2c1]

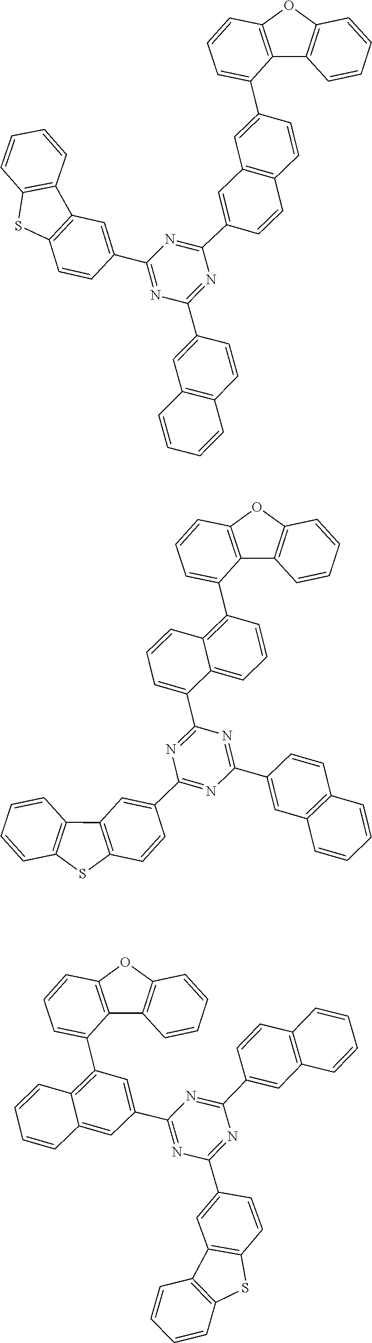 [SMILES: c1ccc2cc(-c3nc(-c4ccc5ccc(-c6cccc7oc8ccccc8c67)cc5c4)nc(-c4ccc5sc6ccccc6c5c4)n3)ccc2c1 ; c1ccc2cc(-c3nc(-c4ccc5sc6ccccc6c5c4)nc(-c4cccc5c(-c6cccc7oc8ccccc8c67)cccc45)n3)ccc2c1 ; c1ccc2cc(-c3nc(-c4cc(-c5cccc6oc7ccccc7c56)c5ccccc5c4)nc(-c4ccc5sc6ccccc6c5c4)n3)ccc2c1]

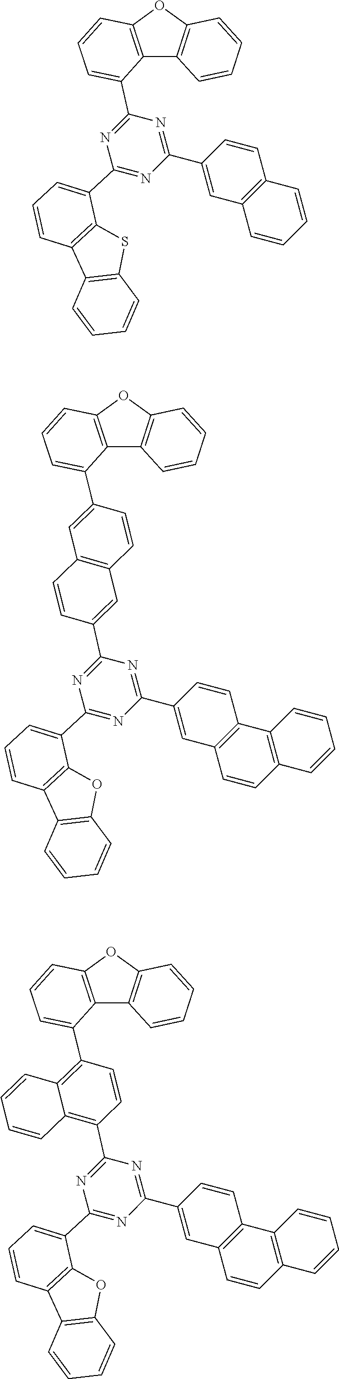 [SMILES: c1ccc2cc(-c3nc(-c4cccc5c4sc4ccccc45)nc(-c4cccc5oc6ccccc6c45)n3)ccc2c1 ; c1ccc2c(c1)ccc1cc(-c3nc(-c4ccc5cc(-c6cccc7oc8ccccc8c67)ccc5c4)nc(-c4cccc5c4oc4ccccc45)n3)ccc12 ; c1ccc2c(c1)ccc1cc(-c3nc(-c4ccc(-c5cccc6oc7ccccc7c56)c5ccccc45)nc(-c4cccc5c4oc4ccccc45)n3)ccc12]

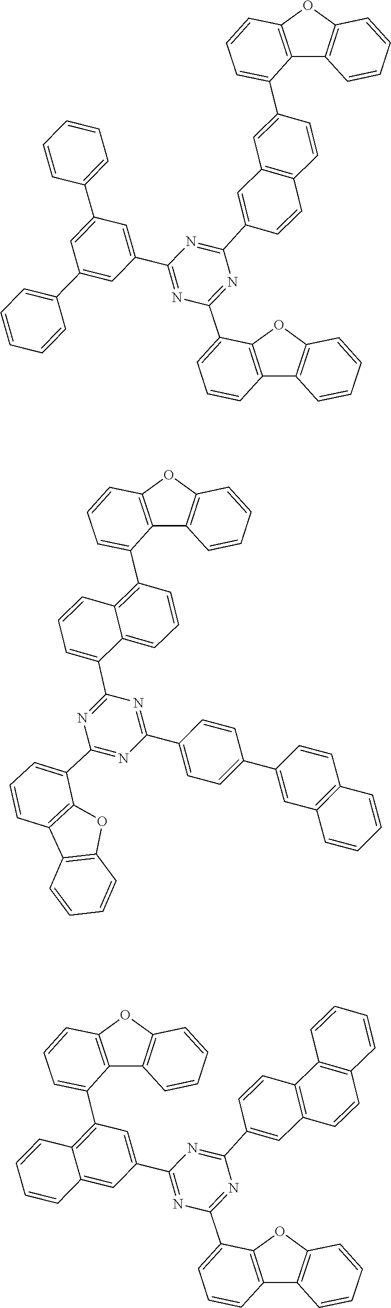 [SMILES: c1ccc(-c2cc(-c3ccccc3)cc(-c3nc(-c4ccc5ccc(-c6cccc7oc8ccccc8c67)cc5c4)nc(-c4cccc5c4oc4ccccc45)n3)c2)cc1 ; c1ccc2cc(-c3ccc(-c4nc(-c5cccc6c(-c7cccc8oc9ccccc9c78)cccc56)nc(-c5cccc6c5oc5ccccc56)n4)cc3)ccc2c1 ; c1ccc2c(-c3cccc4oc5ccccc5c34)cc(-c3nc(-c4ccc5c(ccc6ccccc65)c4)nc(-c4cccc5c4oc4ccccc45)n3)cc2c1]

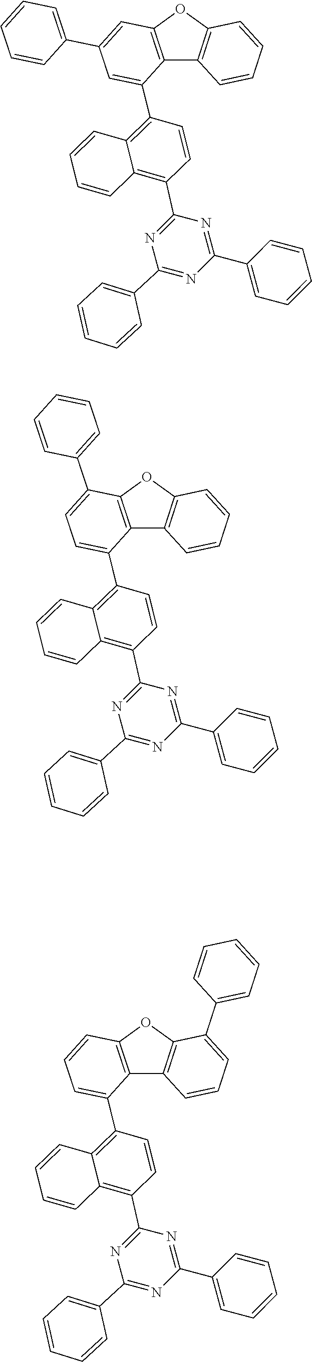 [SMILES: c1ccc(-c2cc(-c3ccc(-c4nc(-c5ccccc5)nc(-c5ccccc5)n4)c4ccccc34)c3c(c2)oc2ccccc23)cc1 ; c1ccc(-c2nc(-c3ccccc3)nc(-c3ccc(-c4ccc(-c5ccccc5)c5oc6ccccc6c45)c4ccccc34)n2)cc1 ; c1ccc(-c2nc(-c3ccccc3)nc(-c3ccc(-c4cccc5oc6c(-c7ccccc7)cccc6c45)c4ccccc34)n2)cc1]

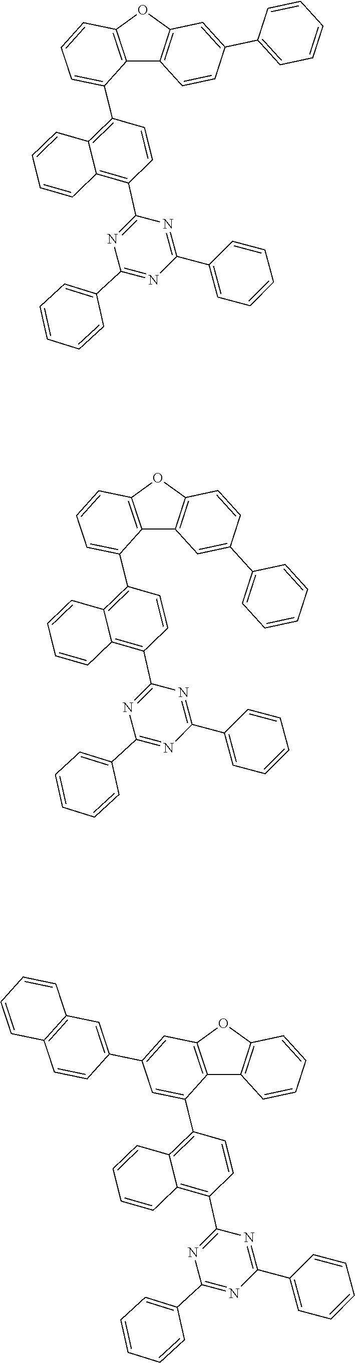 [SMILES: c1ccc(-c2ccc3c(c2)oc2cccc(-c4ccc(-c5nc(-c6ccccc6)nc(-c6ccccc6)n5)c5ccccc45)c23)cc1 ; c1ccc(-c2ccc3oc4cccc(-c5ccc(-c6nc(-c7ccccc7)nc(-c7ccccc7)n6)c6ccccc56)c4c3c2)cc1 ; c1ccc(-c2nc(-c3ccccc3)nc(-c3ccc(-c4cc(-c5ccc6ccccc6c5)cc5oc6ccccc6c45)c4ccccc34)n2)cc1]

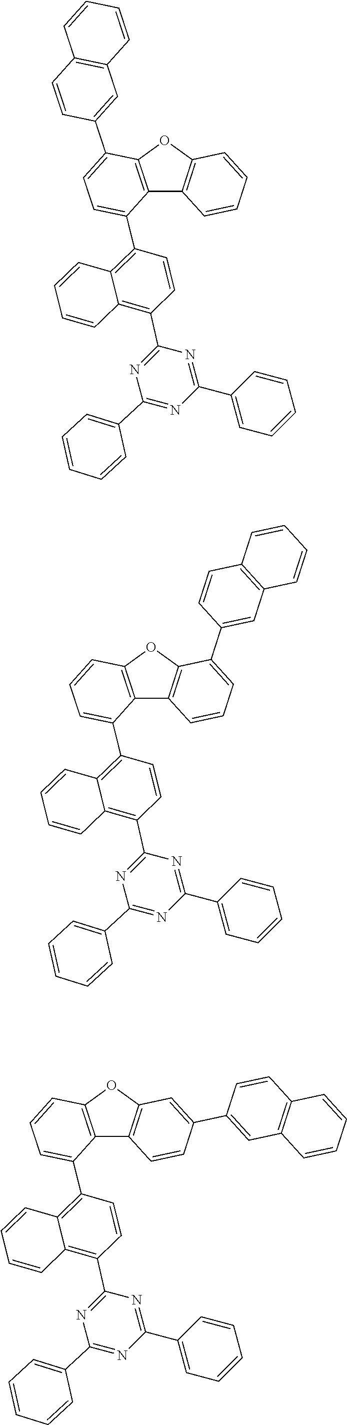 [SMILES: c1ccc(-c2nc(-c3ccccc3)nc(-c3ccc(-c4ccc(-c5ccc6ccccc6c5)c5oc6ccccc6c45)c4ccccc34)n2)cc1 ; c1ccc(-c2nc(-c3ccccc3)nc(-c3ccc(-c4cccc5oc6c(-c7ccc8ccccc8c7)cccc6c45)c4ccccc34)n2)cc1 ; c1ccc(-c2nc(-c3ccccc3)nc(-c3ccc(-c4cccc5oc6cc(-c7ccc8ccccc8c7)ccc6c45)c4ccccc34)n2)cc1]

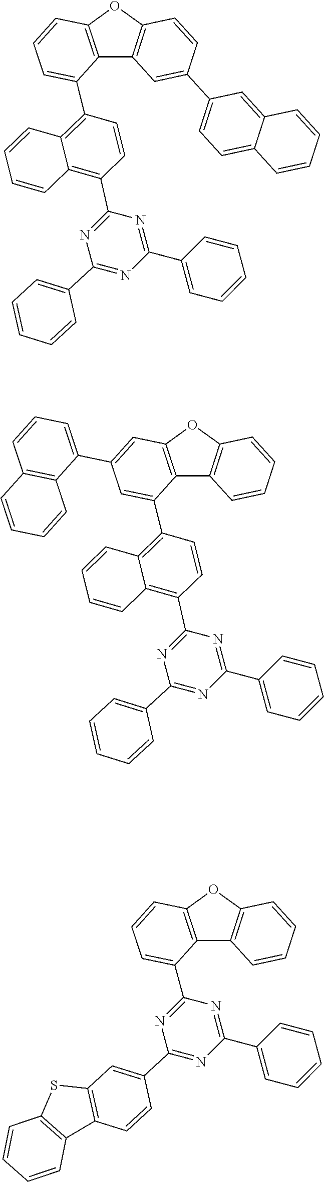 [SMILES: c1ccc(-c2nc(-c3ccccc3)nc(-c3ccc(-c4cccc5oc6ccc(-c7ccc8ccccc8c7)cc6c45)c4ccccc34)n2)cc1 ; c1ccc(-c2nc(-c3ccccc3)nc(-c3ccc(-c4cc(-c5cccc6ccccc56)cc5oc6ccccc6c45)c4ccccc34)n2)cc1 ; c1ccc(-c2nc(-c3ccc4c(c3)sc3ccccc34)nc(-c3cccc4oc5ccccc5c34)n2)cc1]

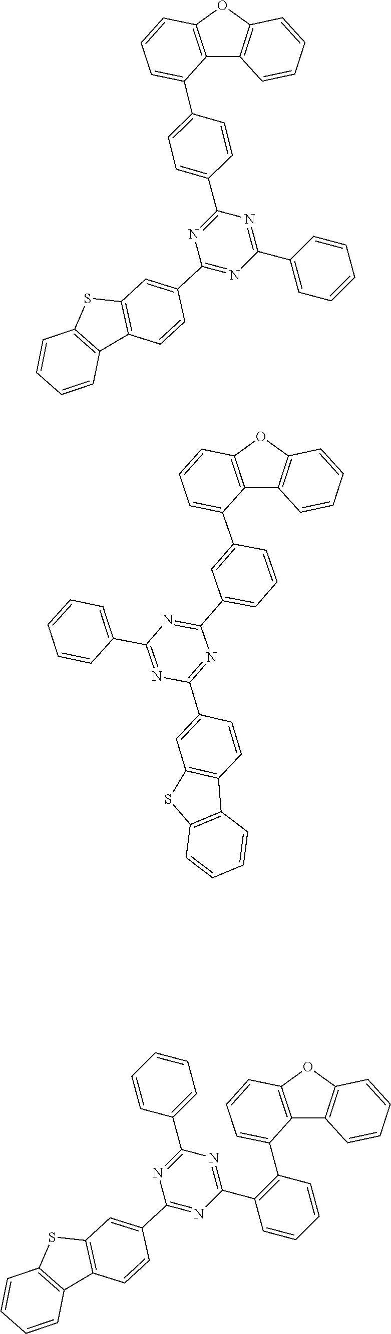 [SMILES: c1ccc(-c2nc(-c3ccc(-c4cccc5oc6ccccc6c45)cc3)nc(-c3ccc4c(c3)sc3ccccc34)n2)cc1 ; c1ccc(-c2nc(-c3cccc(-c4cccc5oc6ccccc6c45)c3)nc(-c3ccc4c(c3)sc3ccccc34)n2)cc1 ; c1ccc(-c2nc(-c3ccc4c(c3)sc3ccccc34)nc(-c3ccccc3-c3cccc4oc5ccccc5c34)n2)cc1]

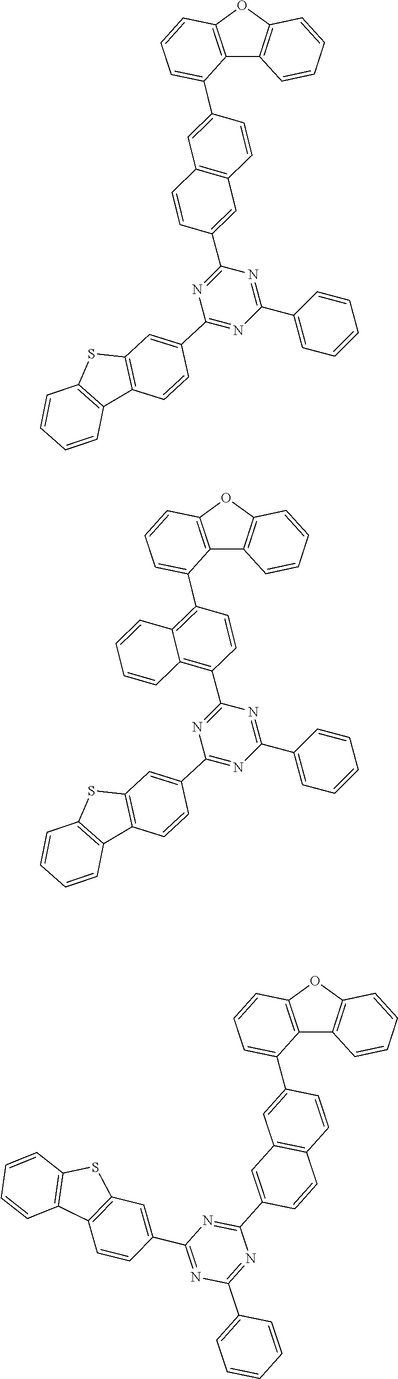 [SMILES: c1ccc(-c2nc(-c3ccc4cc(-c5cccc6oc7ccccc7c56)ccc4c3)nc(-c3ccc4c(c3)sc3ccccc34)n2)cc1 ; c1ccc(-c2nc(-c3ccc4c(c3)sc3ccccc34)nc(-c3ccc(-c4cccc5oc6ccccc6c45)c4ccccc34)n2)cc1 ; c1ccc(-c2nc(-c3ccc4ccc(-c5cccc6oc7ccccc7c56)cc4c3)nc(-c3ccc4c(c3)sc3ccccc34)n2)cc1]

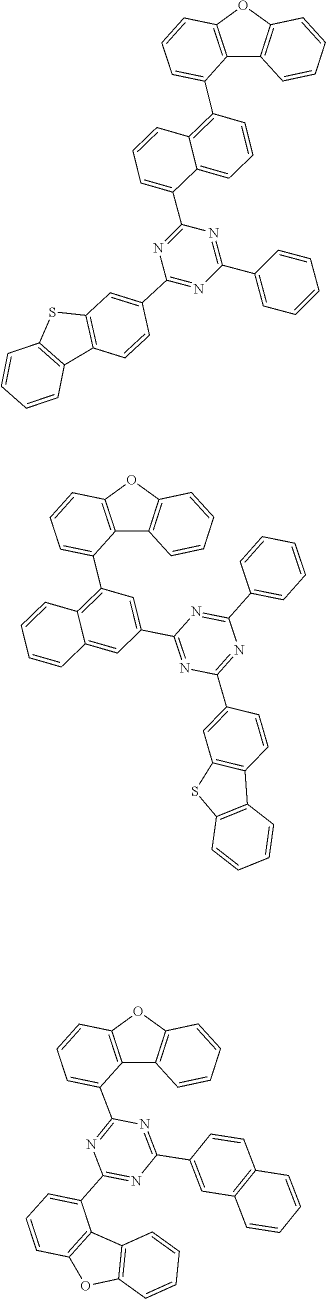 [SMILES: c1ccc(-c2nc(-c3ccc4c(c3)sc3ccccc34)nc(-c3cccc4c(-c5cccc6oc7ccccc7c56)cccc34)n2)cc1 ; c1ccc(-c2nc(-c3cc(-c4cccc5oc6ccccc6c45)c4ccccc4c3)nc(-c3ccc4c(c3)sc3ccccc34)n2)cc1 ; c1ccc2cc(-c3nc(-c4cccc5oc6ccccc6c45)nc(-c4cccc5oc6ccccc6c45)n3)ccc2c1]

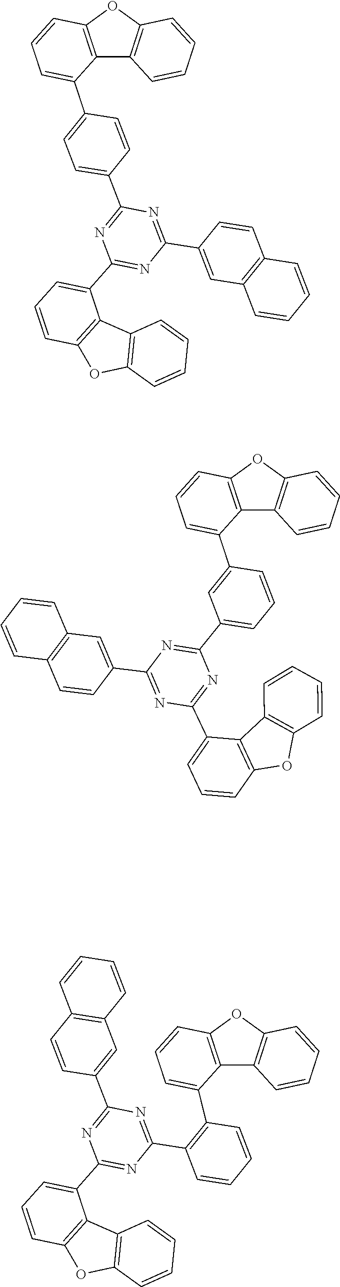 [SMILES: c1ccc2cc(-c3nc(-c4ccc(-c5cccc6oc7ccccc7c56)cc4)nc(-c4cccc5oc6ccccc6c45)n3)ccc2c1 ; c1cc(-c2nc(-c3ccc4ccccc4c3)nc(-c3cccc4oc5ccccc5c34)n2)cc(-c2cccc3oc4ccccc4c23)c1 ; c1ccc(-c2cccc3oc4ccccc4c23)c(-c2nc(-c3ccc4ccccc4c3)nc(-c3cccc4oc5ccccc5c34)n2)c1]

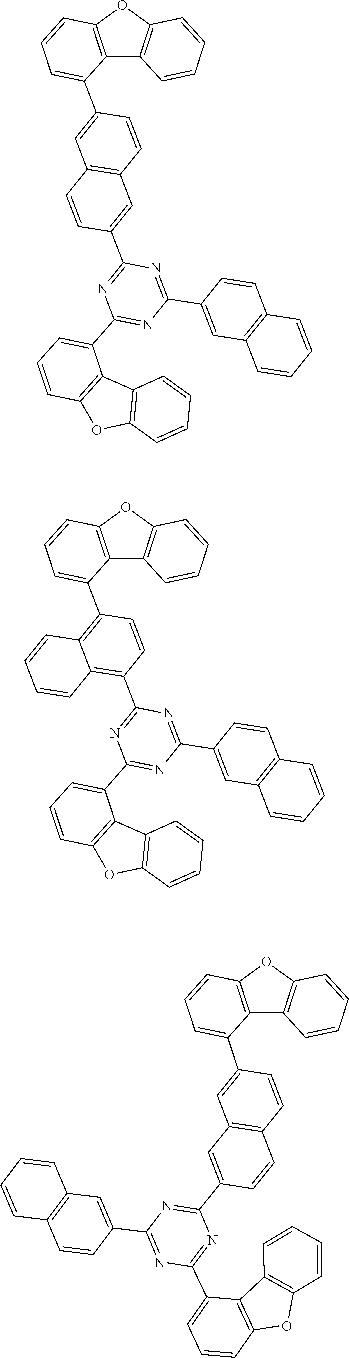 [SMILES: c1ccc2cc(-c3nc(-c4ccc5cc(-c6cccc7oc8ccccc8c67)ccc5c4)nc(-c4cccc5oc6ccccc6c45)n3)ccc2c1 ; c1ccc2cc(-c3nc(-c4ccc(-c5cccc6oc7ccccc7c56)c5ccccc45)nc(-c4cccc5oc6ccccc6c45)n3)ccc2c1 ; c1ccc2cc(-c3nc(-c4ccc5ccc(-c6cccc7oc8ccccc8c67)cc5c4)nc(-c4cccc5oc6ccccc6c45)n3)ccc2c1]

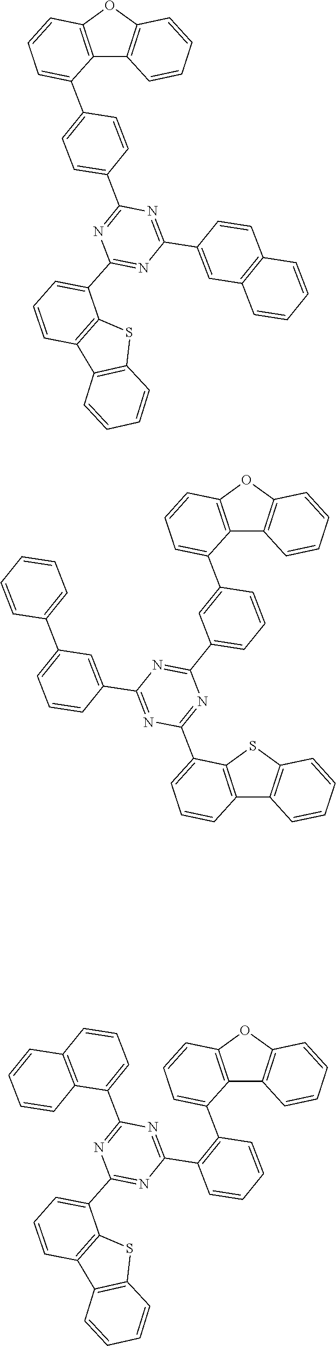 [SMILES: c1ccc2cc(-c3nc(-c4ccc(-c5cccc6oc7ccccc7c56)cc4)nc(-c4cccc5c4sc4ccccc45)n3)ccc2c1 ; c1ccc(-c2cccc(-c3nc(-c4cccc(-c5cccc6oc7ccccc7c56)c4)nc(-c4cccc5c4sc4ccccc45)n3)c2)cc1 ; c1ccc(-c2cccc3oc4ccccc4c23)c(-c2nc(-c3cccc4ccccc34)nc(-c3cccc4c3sc3ccccc34)n2)c1]

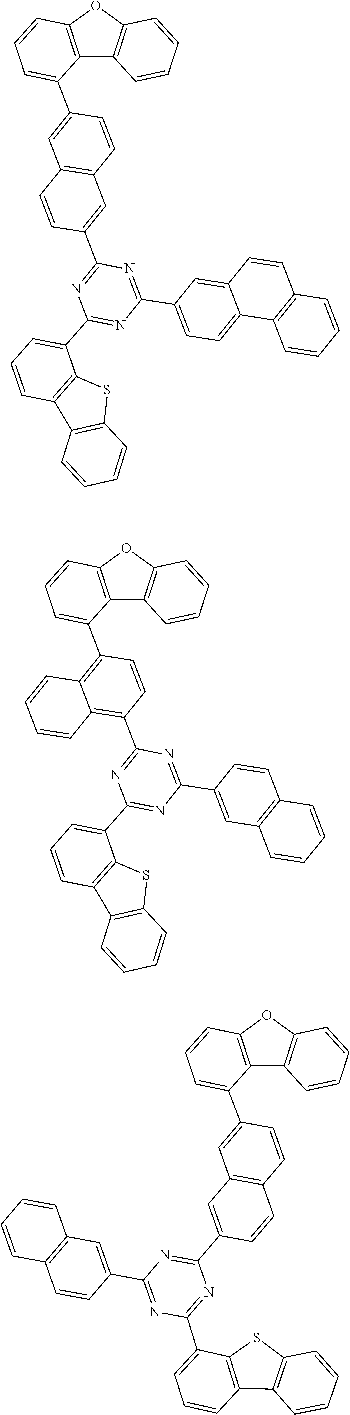 [SMILES: c1ccc2c(c1)ccc1cc(-c3nc(-c4ccc5cc(-c6cccc7oc8ccccc8c67)ccc5c4)nc(-c4cccc5c4sc4ccccc45)n3)ccc12 ; c1ccc2cc(-c3nc(-c4ccc(-c5cccc6oc7ccccc7c56)c5ccccc45)nc(-c4cccc5c4sc4ccccc45)n3)ccc2c1 ; c1ccc2cc(-c3nc(-c4ccc5ccc(-c6cccc7oc8ccccc8c67)cc5c4)nc(-c4cccc5c4sc4ccccc45)n3)ccc2c1]

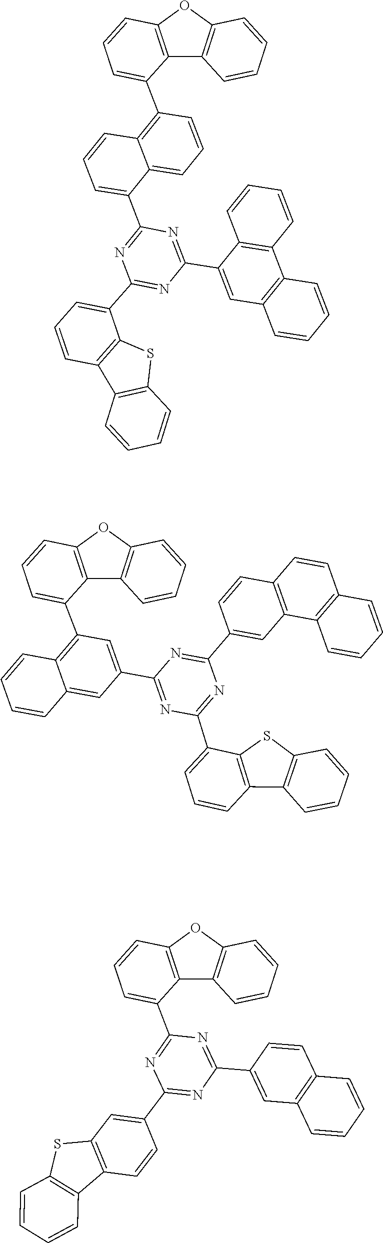 [SMILES: c1ccc2c(c1)cc(-c1nc(-c3cccc4c(-c5cccc6oc7ccccc7c56)cccc34)nc(-c3cccc4c3sc3ccccc34)n1)c1ccccc12 ; c1ccc2c(-c3cccc4oc5ccccc5c34)cc(-c3nc(-c4ccc5ccc6ccccc6c5c4)nc(-c4cccc5c4sc4ccccc45)n3)cc2c1 ; c1ccc2cc(-c3nc(-c4ccc5c(c4)sc4ccccc45)nc(-c4cccc5oc6ccccc6c45)n3)ccc2c1]

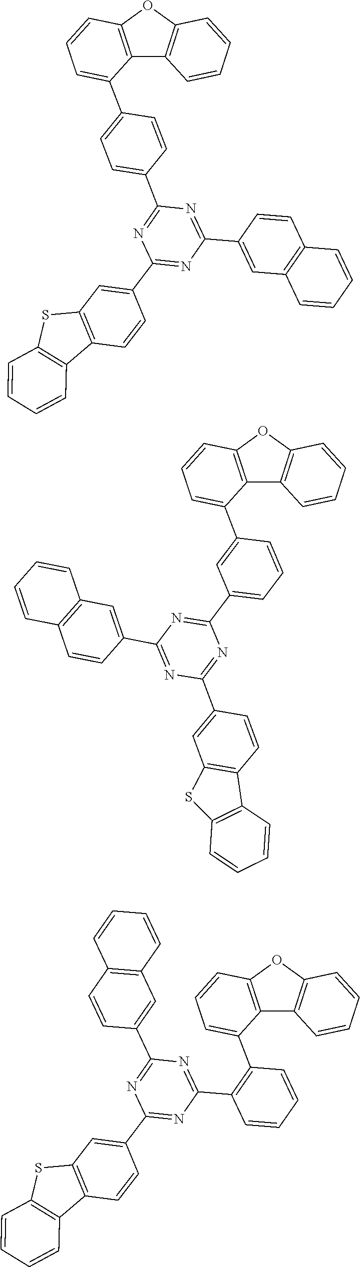 [SMILES: c1ccc2cc(-c3nc(-c4ccc(-c5cccc6oc7ccccc7c56)cc4)nc(-c4ccc5c(c4)sc4ccccc45)n3)ccc2c1 ; c1cc(-c2nc(-c3ccc4ccccc4c3)nc(-c3ccc4c(c3)sc3ccccc34)n2)cc(-c2cccc3oc4ccccc4c23)c1 ; c1ccc(-c2cccc3oc4ccccc4c23)c(-c2nc(-c3ccc4ccccc4c3)nc(-c3ccc4c(c3)sc3ccccc34)n2)c1]

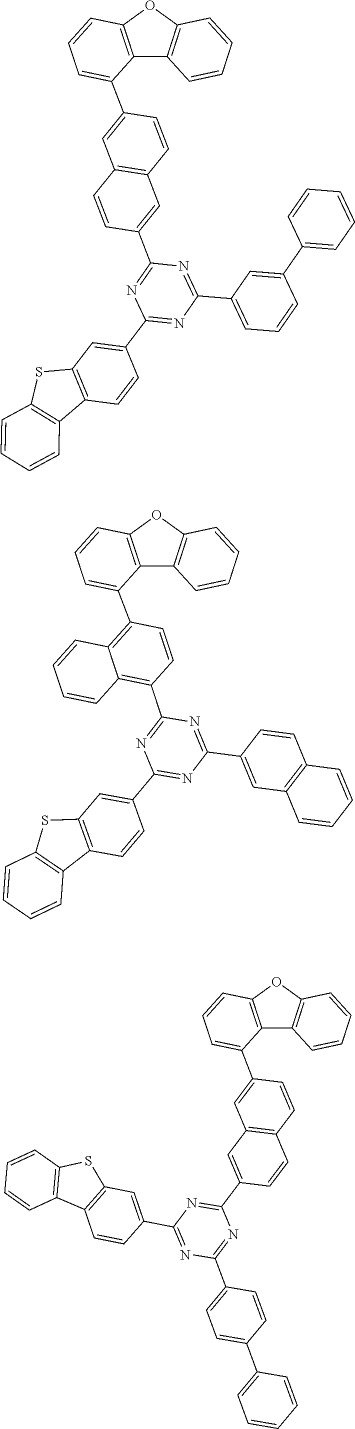 [SMILES: c1ccc(-c2cccc(-c3nc(-c4ccc5cc(-c6cccc7oc8ccccc8c67)ccc5c4)nc(-c4ccc5c(c4)sc4ccccc45)n3)c2)cc1 ; c1ccc2cc(-c3nc(-c4ccc5c(c4)sc4ccccc45)nc(-c4ccc(-c5cccc6oc7ccccc7c56)c5ccccc45)n3)ccc2c1 ; c1ccc(-c2ccc(-c3nc(-c4ccc5ccc(-c6cccc7oc8ccccc8c67)cc5c4)nc(-c4ccc5c(c4)sc4ccccc45)n3)cc2)cc1]

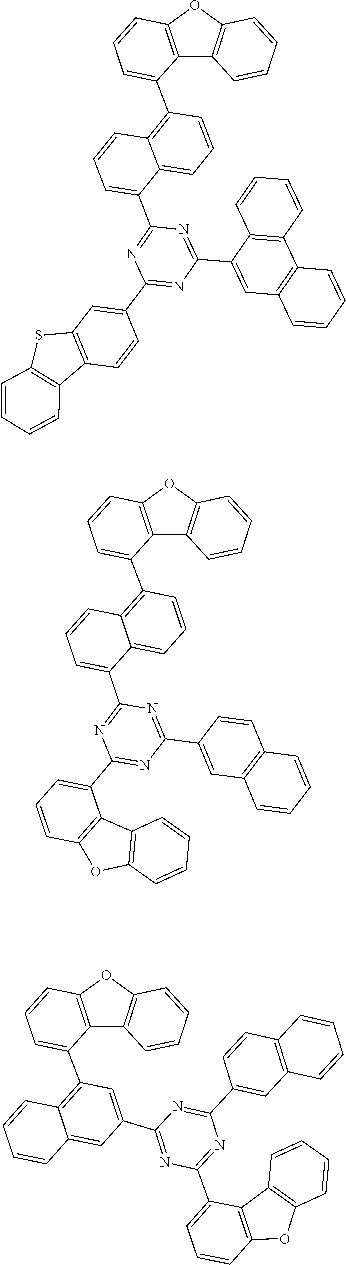 [SMILES: c1ccc2c(c1)cc(-c1nc(-c3ccc4c(c3)sc3ccccc34)nc(-c3cccc4c(-c5cccc6oc7ccccc7c56)cccc34)n1)c1ccccc12 ; c1ccc2cc(-c3nc(-c4cccc5c(-c6cccc7oc8ccccc8c67)cccc45)nc(-c4cccc5oc6ccccc6c45)n3)ccc2c1 ; c1ccc2cc(-c3nc(-c4cc(-c5cccc6oc7ccccc7c56)c5ccccc5c4)nc(-c4cccc5oc6ccccc6c45)n3)ccc2c1]

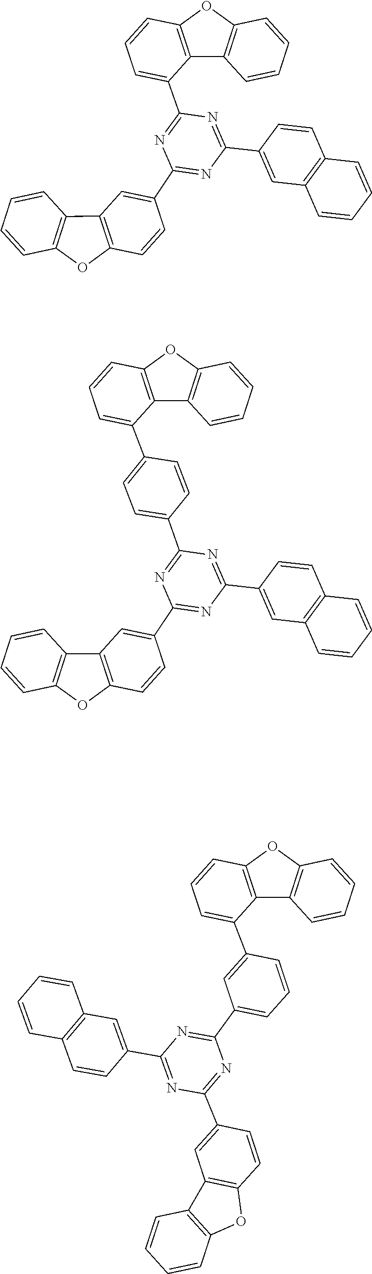 [SMILES: c1ccc2cc(-c3nc(-c4ccc5oc6ccccc6c5c4)nc(-c4cccc5oc6ccccc6c45)n3)ccc2c1 ; c1ccc2cc(-c3nc(-c4ccc(-c5cccc6oc7ccccc7c56)cc4)nc(-c4ccc5oc6ccccc6c5c4)n3)ccc2c1 ; c1cc(-c2nc(-c3ccc4ccccc4c3)nc(-c3ccc4oc5ccccc5c4c3)n2)cc(-c2cccc3oc4ccccc4c23)c1]

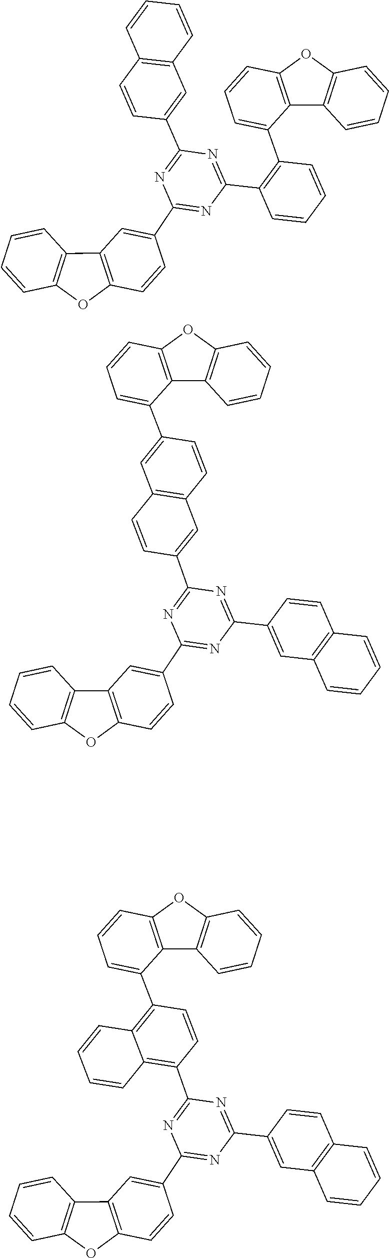 [SMILES: c1ccc(-c2cccc3oc4ccccc4c23)c(-c2nc(-c3ccc4ccccc4c3)nc(-c3ccc4oc5ccccc5c4c3)n2)c1 ; c1ccc2cc(-c3nc(-c4ccc5cc(-c6cccc7oc8ccccc8c67)ccc5c4)nc(-c4ccc5oc6ccccc6c5c4)n3)ccc2c1 ; c1ccc2cc(-c3nc(-c4ccc5oc6ccccc6c5c4)nc(-c4ccc(-c5cccc6oc7ccccc7c56)c5ccccc45)n3)ccc2c1]

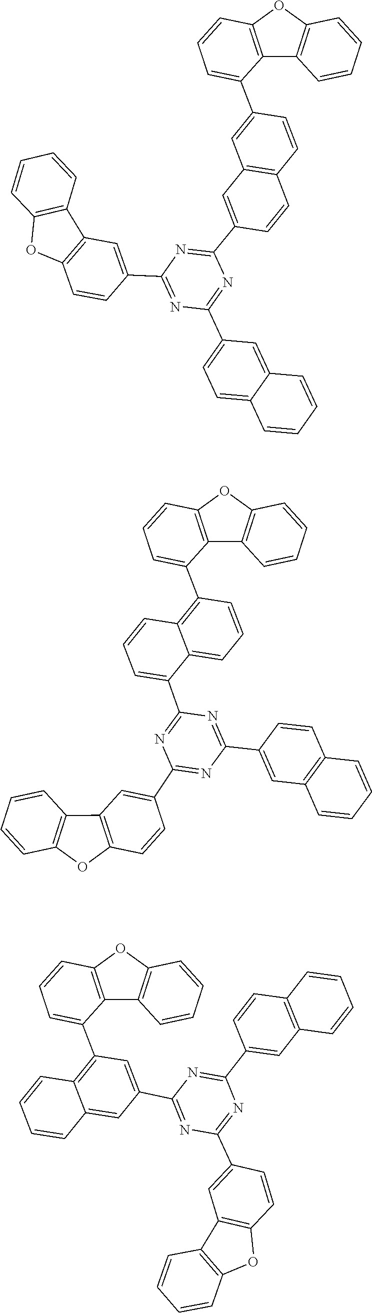 [SMILES: c1ccc2cc(-c3nc(-c4ccc5ccc(-c6cccc7oc8ccccc8c67)cc5c4)nc(-c4ccc5oc6ccccc6c5c4)n3)ccc2c1 ; c1ccc2cc(-c3nc(-c4ccc5oc6ccccc6c5c4)nc(-c4cccc5c(-c6cccc7oc8ccccc8c67)cccc45)n3)ccc2c1 ; c1ccc2cc(-c3nc(-c4cc(-c5cccc6oc7ccccc7c56)c5ccccc5c4)nc(-c4ccc5oc6ccccc6c5c4)n3)ccc2c1]

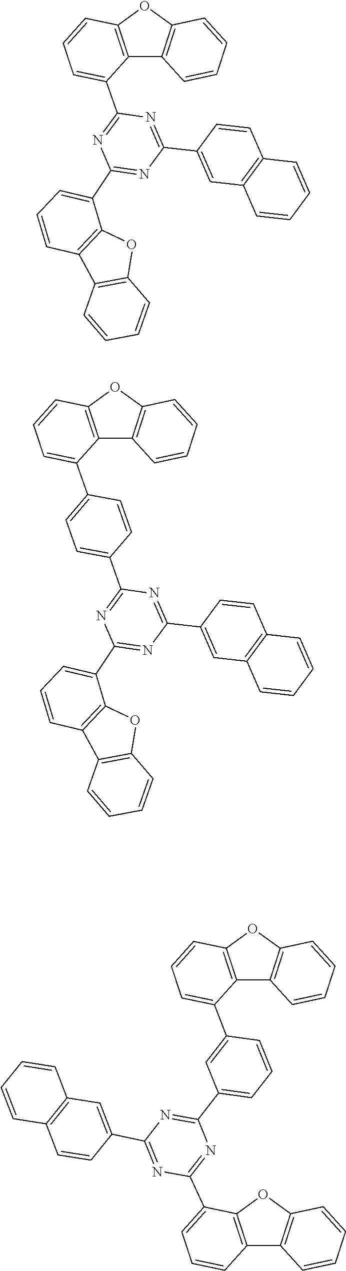 [SMILES: c1ccc2cc(-c3nc(-c4cccc5c4oc4ccccc45)nc(-c4cccc5oc6ccccc6c45)n3)ccc2c1 ; c1ccc2cc(-c3nc(-c4ccc(-c5cccc6oc7ccccc7c56)cc4)nc(-c4cccc5c4oc4ccccc45)n3)ccc2c1 ; c1cc(-c2nc(-c3ccc4ccccc4c3)nc(-c3cccc4c3oc3ccccc34)n2)cc(-c2cccc3oc4ccccc4c23)c1]

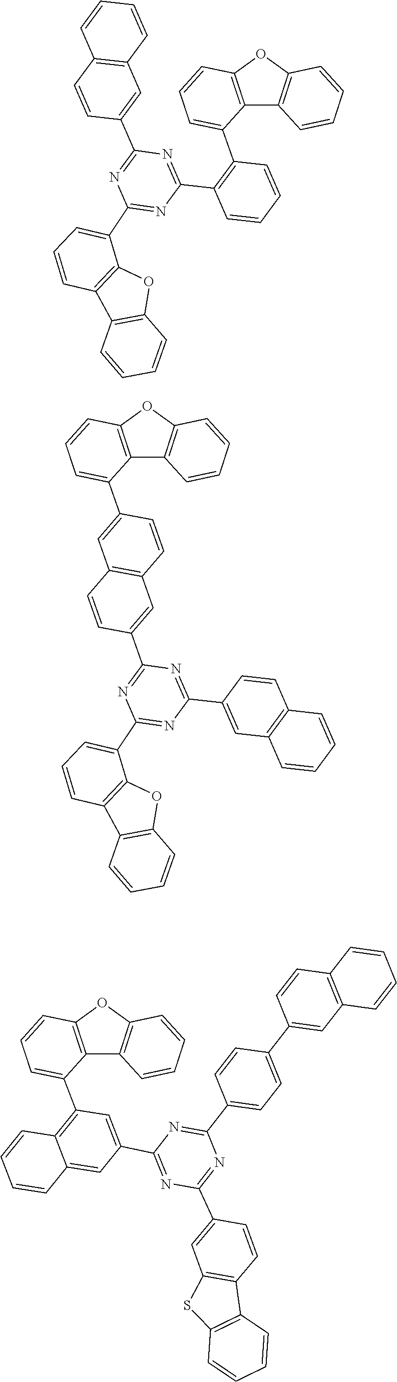

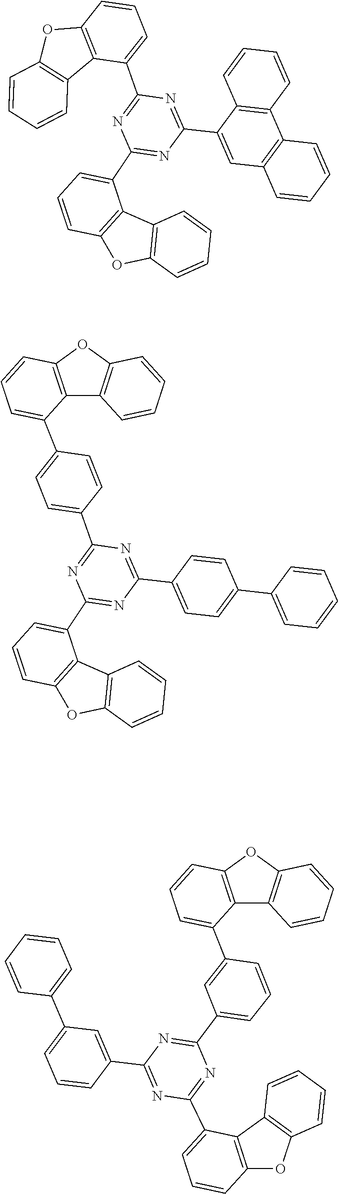 [SMILES: c1ccc2c(c1)cc(-c1nc(-c3cccc4oc5ccccc5c34)nc(-c3cccc4oc5ccccc5c34)n1)c1ccccc12 ; c1ccc(-c2ccc(-c3nc(-c4ccc(-c5cccc6oc7ccccc7c56)cc4)nc(-c4cccc5oc6ccccc6c45)n3)cc2)cc1 ; c1ccc(-c2cccc(-c3nc(-c4cccc(-c5cccc6oc7ccccc7c56)c4)nc(-c4cccc5oc6ccccc6c45)n3)c2)cc1]

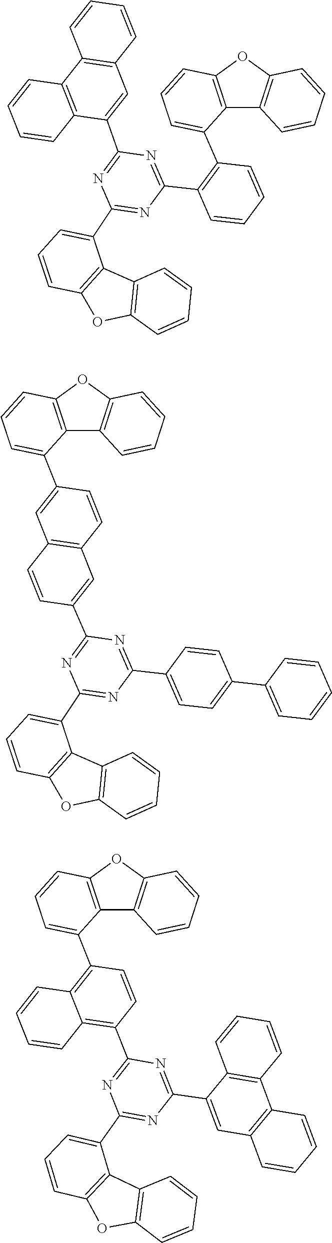

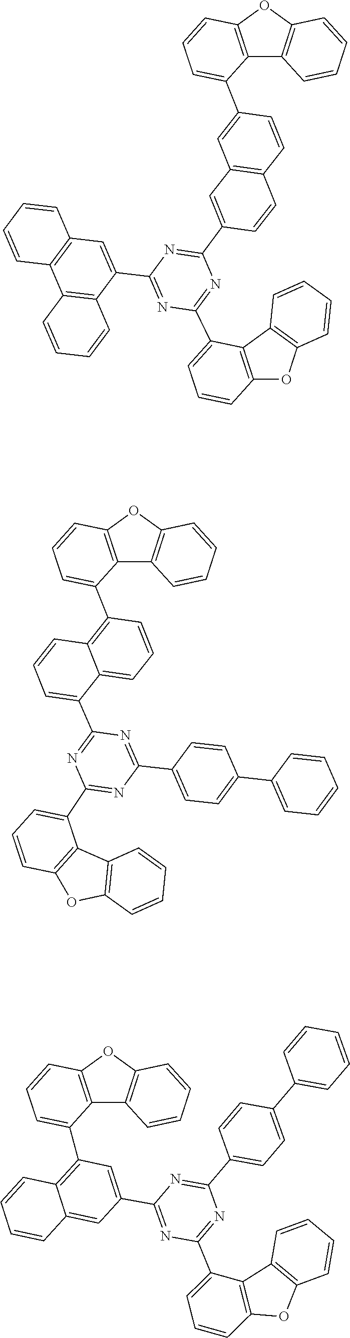 [SMILES: c1ccc2c(c1)cc(-c1nc(-c3ccc4ccc(-c5cccc6oc7ccccc7c56)cc4c3)nc(-c3cccc4oc5ccccc5c34)n1)c1ccccc12 ; c1ccc(-c2ccc(-c3nc(-c4cccc5c(-c6cccc7oc8ccccc8c67)cccc45)nc(-c4cccc5oc6ccccc6c45)n3)cc2)cc1 ; c1ccc(-c2ccc(-c3nc(-c4cc(-c5cccc6oc7ccccc7c56)c5ccccc5c4)nc(-c4cccc5oc6ccccc6c45)n3)cc2)cc1]

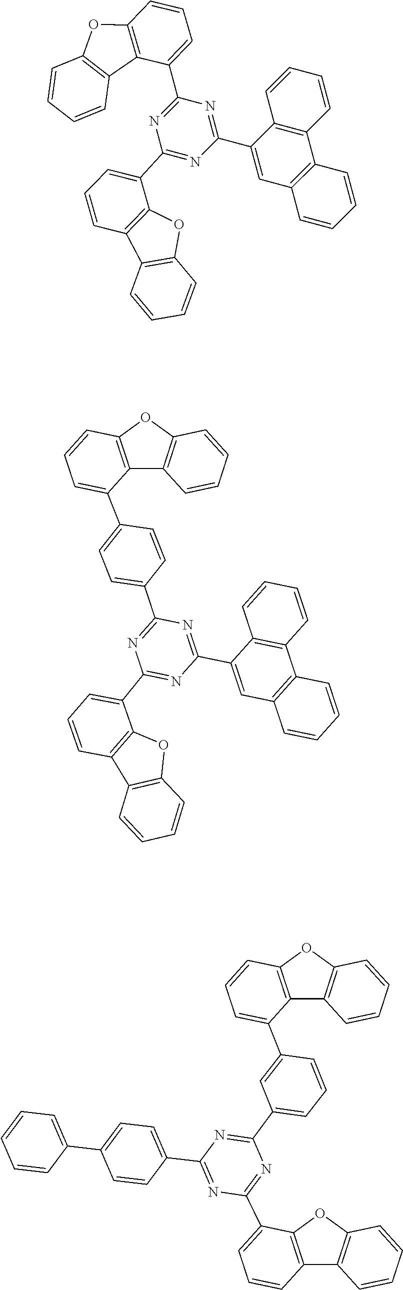 [SMILES: c1ccc2c(c1)cc(-c1nc(-c3cccc4c3oc3ccccc34)nc(-c3cccc4oc5ccccc5c34)n1)c1ccccc12 ; c1ccc2c(c1)cc(-c1nc(-c3ccc(-c4cccc5oc6ccccc6c45)cc3)nc(-c3cccc4c3oc3ccccc34)n1)c1ccccc12 ; c1ccc(-c2ccc(-c3nc(-c4cccc(-c5cccc6oc7ccccc7c56)c4)nc(-c4cccc5c4oc4ccccc45)n3)cc2)cc1]

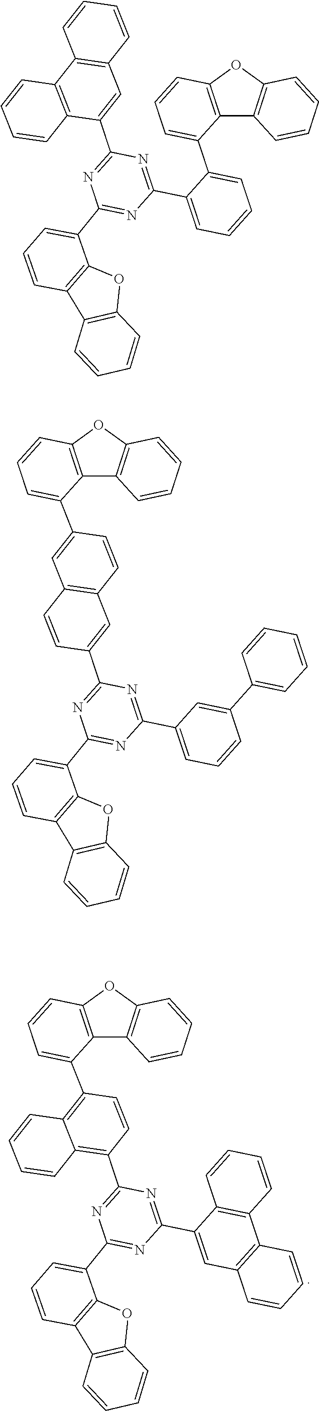 [SMILES: c1ccc(-c2cccc3oc4ccccc4c23)c(-c2nc(-c3cc4ccccc4c4ccccc34)nc(-c3cccc4c3oc3ccccc34)n2)c1 ; c1ccc(-c2cccc(-c3nc(-c4ccc5cc(-c6cccc7oc8ccccc8c67)ccc5c4)nc(-c4cccc5c4oc4ccccc45)n3)c2)cc1 ; c1ccc2c(c1)cc(-c1nc(-c3ccc(-c4cccc5oc6ccccc6c45)c4ccccc34)nc(-c3cccc4c3oc3ccccc34)n1)c1ccccc12]

7. The organic light emitting device of claim 1,
wherein Chemical Formula 2 is any one selected from the group consisting of the following Chemical Formulae 2-1 to 2-3: 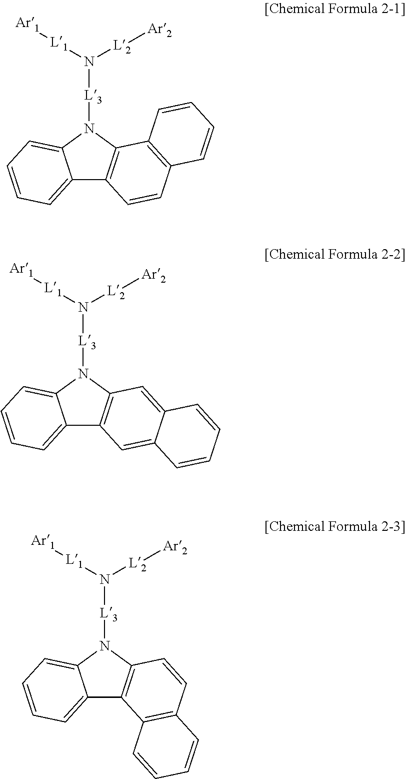
wherein in the Chemical Formulae 2-1 to 2-3:
Ar′₁, Ar′₂, and L′₁ to L′₃ are as defined for Chemical Formula 2.

8. The organic light emitting device of claim 1,
wherein L′₃ is any one selected from the group consisting of the following structural formulae:

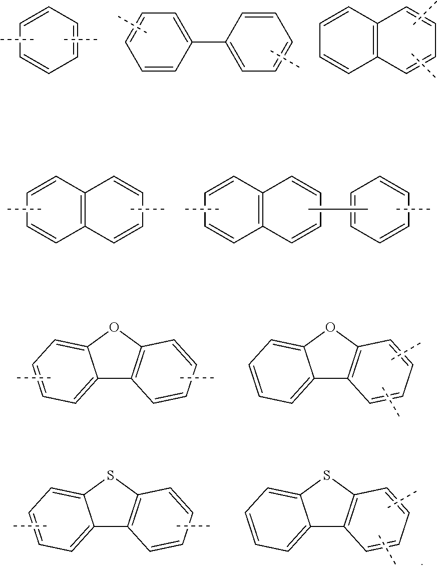

9. The organic light emitting device of claim 1,
wherein L′₁ and L′₂ are each independently a single bond or phenylene.

10. The organic light emitting device of claim 1,
wherein Ar′₁ and Ar′₂ are each independently phenyl, biphenylyl, terphenylyl, naphthyl, phenanthrenyl, dibenzofuranyl, dibenzothiophenyl, or benzonaphthofuranyl.

11. The organic light emitting device of claim 1,
wherein the compound of Chemical Formula 2 is any one compound selected from the group consisting of the following compounds: 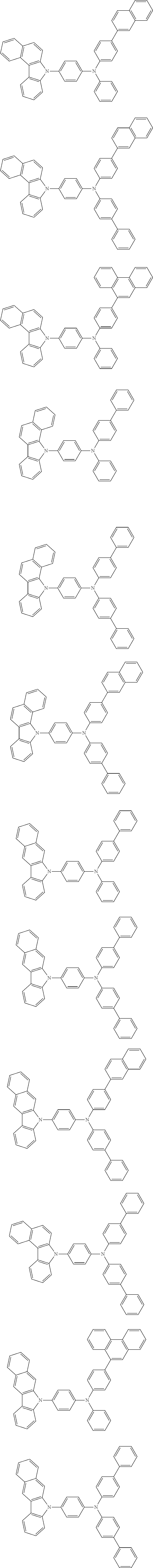 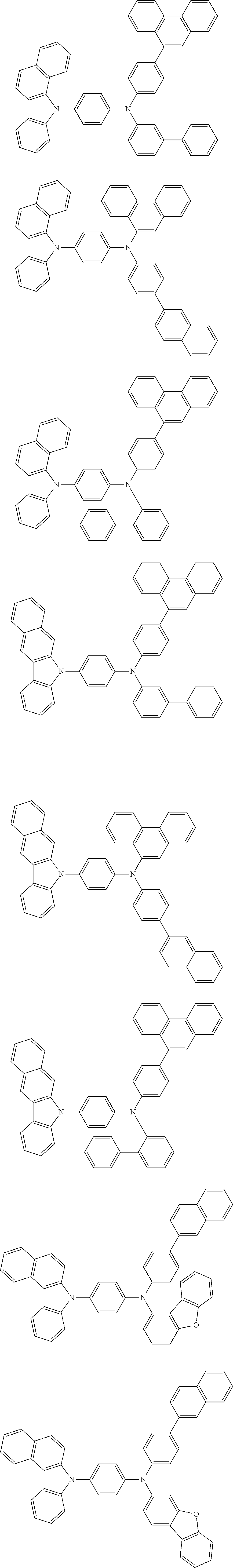 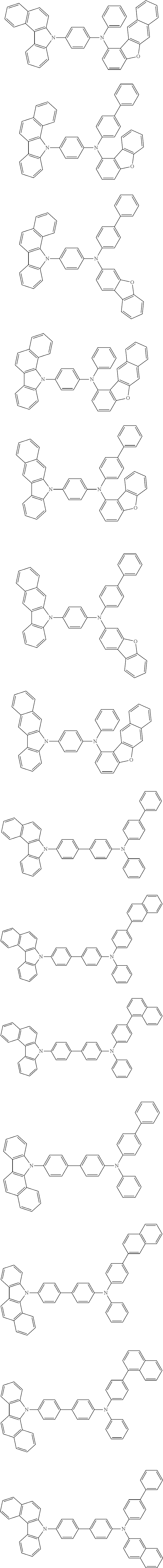 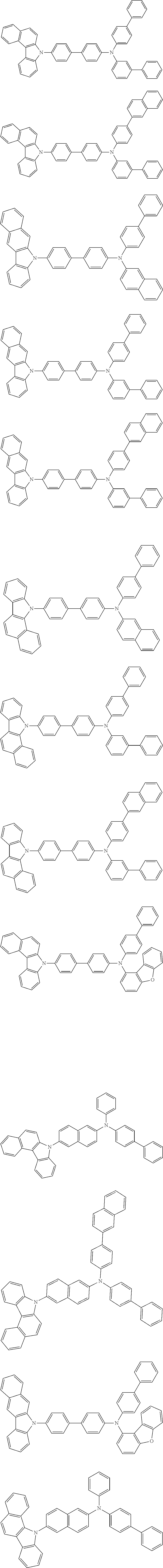 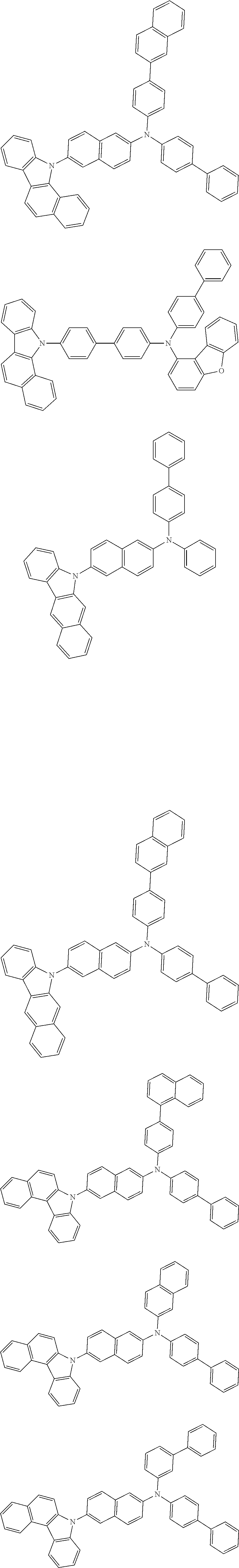 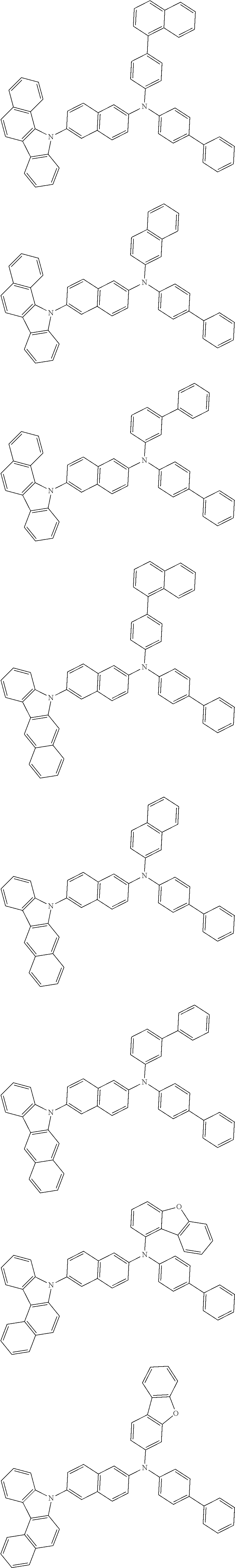 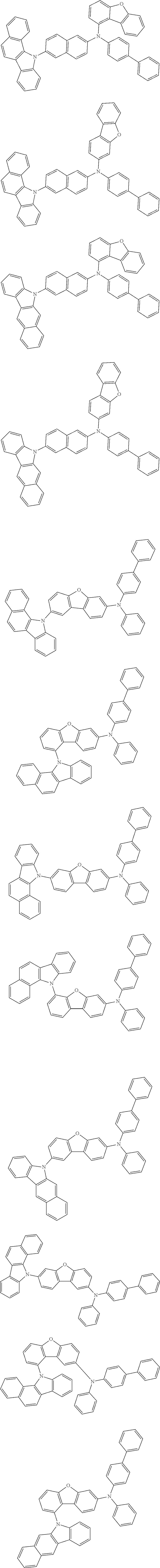 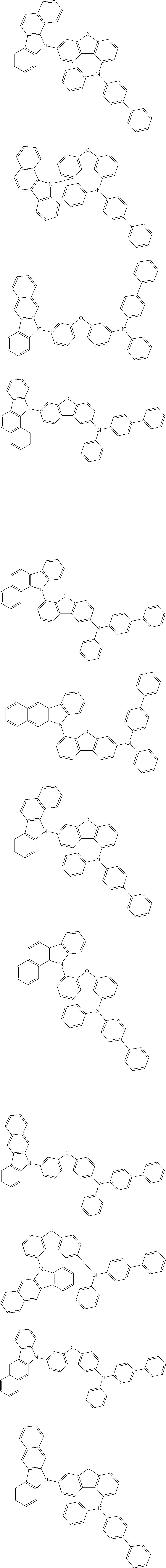 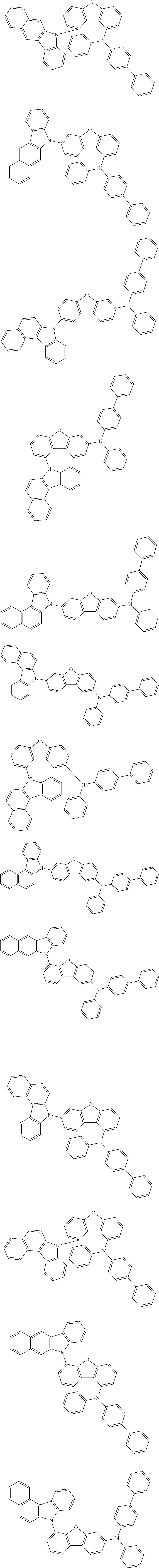 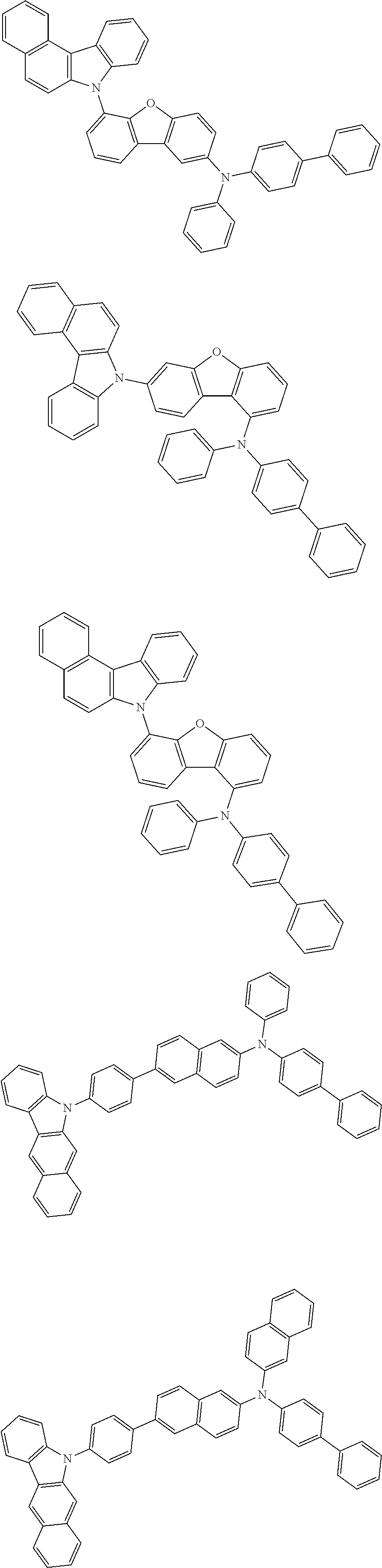 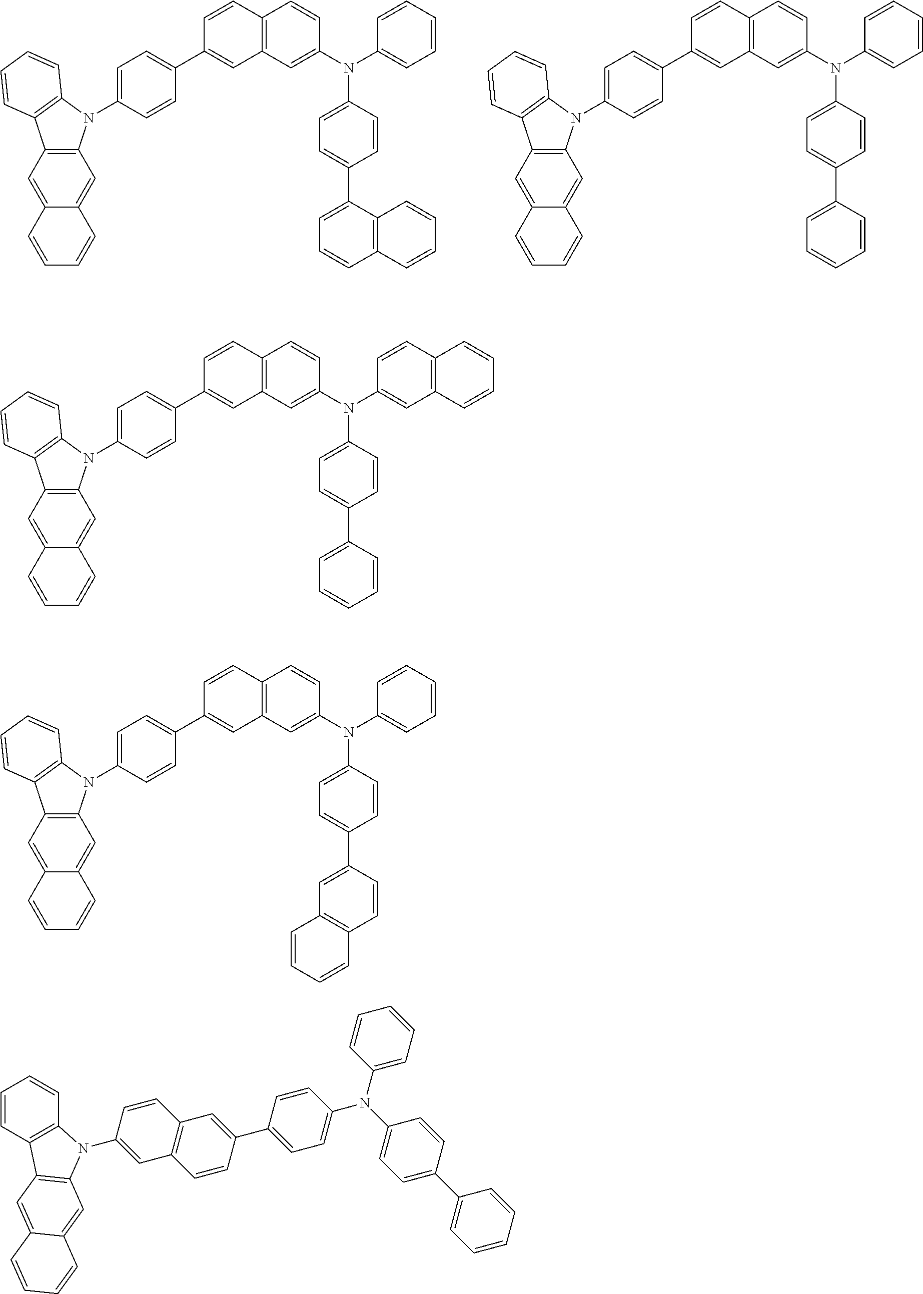 [SMILES: c1ccc(N(c2ccc(-c3cccc4ccccc34)cc2)c2ccc3ccc(-c4ccc(-n5c6ccccc6c6cc7ccccc7cc65)cc4)cc3c2)cc1 ; c1ccc(-c2ccc(N(c3ccccc3)c3ccc4ccc(-c5ccc(-n6c7ccccc7c7cc8ccccc8cc76)cc5)cc4c3)cc2)cc1 ; c1ccc(-c2ccc(N(c3ccc4ccccc4c3)c3ccc4ccc(-c5ccc(-n6c7ccccc7c7cc8ccccc8cc76)cc5)cc4c3)cc2)cc1 ; c1ccc(N(c2ccc(-c3ccc4ccccc4c3)cc2)c2ccc3ccc(-c4ccc(-n5c6ccccc6c6cc7ccccc7cc65)cc4)cc3c2)cc1 ; c1ccc(-c2ccc(N(c3ccccc3)c3ccc(-c4ccc5cc(-n6c7ccccc7c7cc8ccccc8cc76)ccc5c4)cc3)cc2)cc1] 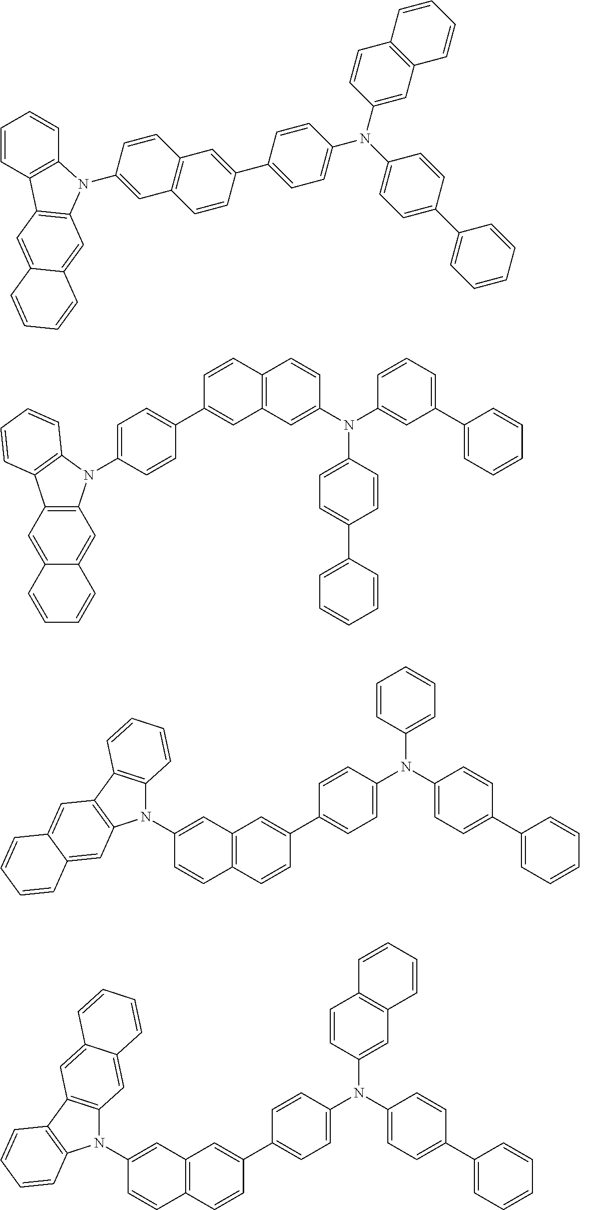 [SMILES: c1ccc(-c2ccc(N(c3ccc(-c4ccc5cc(-n6c7ccccc7c7cc8ccccc8cc76)ccc5c4)cc3)c3ccc4ccccc4c3)cc2)cc1 ; c1ccc(-c2ccc(N(c3cccc(-c4ccccc4)c3)c3ccc4ccc(-c5ccc(-n6c7ccccc7c7cc8ccccc8cc76)cc5)cc4c3)cc2)cc1 ; c1ccc(-c2ccc(N(c3ccccc3)c3ccc(-c4ccc5ccc(-n6c7ccccc7c7cc8ccccc8cc76)cc5c4)cc3)cc2)cc1 ; c1ccc(-c2ccc(N(c3ccc(-c4ccc5ccc(-n6c7ccccc7c7cc8ccccc8cc76)cc5c4)cc3)c3ccc4ccccc4c3)cc2)cc1] 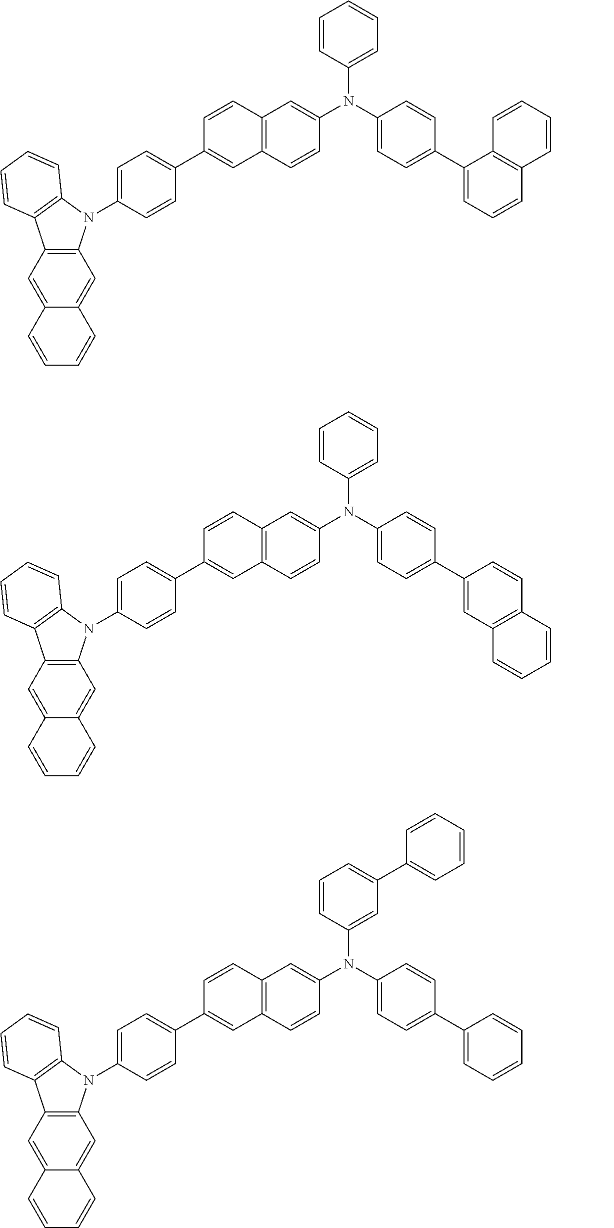 [SMILES: c1ccc(N(c2ccc(-c3cccc4ccccc34)cc2)c2ccc3cc(-c4ccc(-n5c6ccccc6c6cc7ccccc7cc65)cc4)ccc3c2)cc1 ; c1ccc(N(c2ccc(-c3ccc4ccccc4c3)cc2)c2ccc3cc(-c4ccc(-n5c6ccccc6c6cc7ccccc7cc65)cc4)ccc3c2)cc1 ; c1ccc(-c2ccc(N(c3cccc(-c4ccccc4)c3)c3ccc4cc(-c5ccc(-n6c7ccccc7c7cc8ccccc8cc76)cc5)ccc4c3)cc2)cc1] 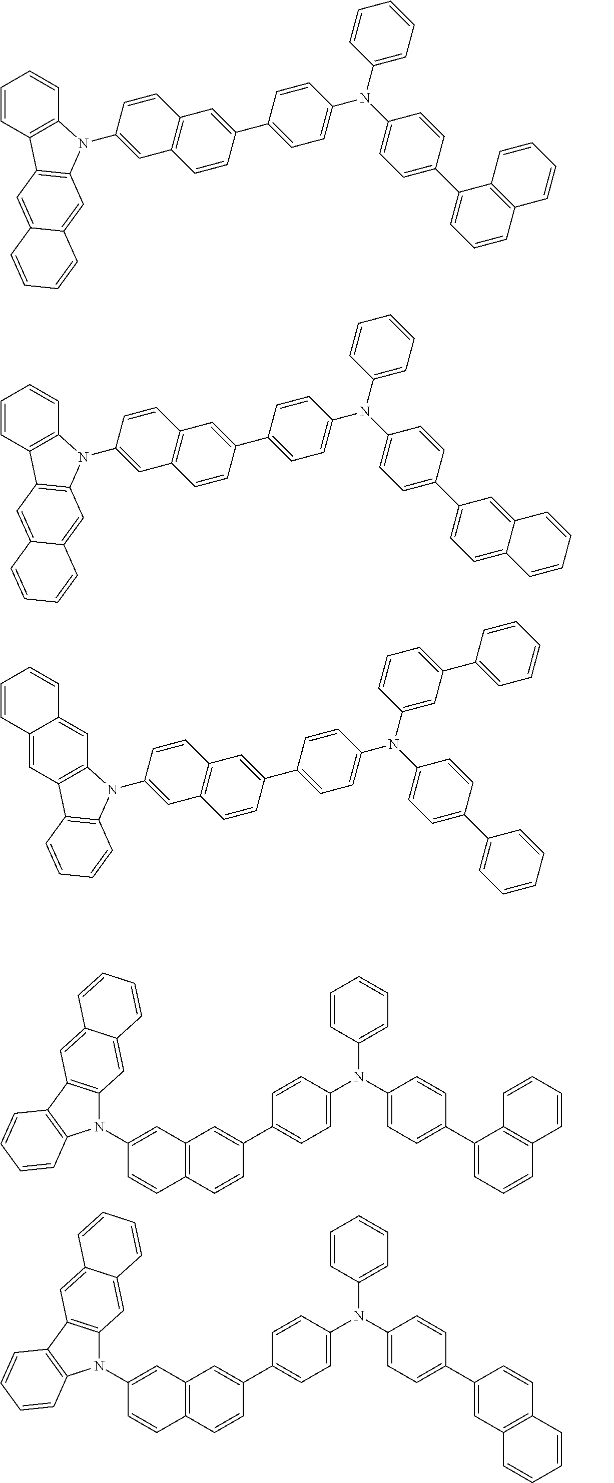 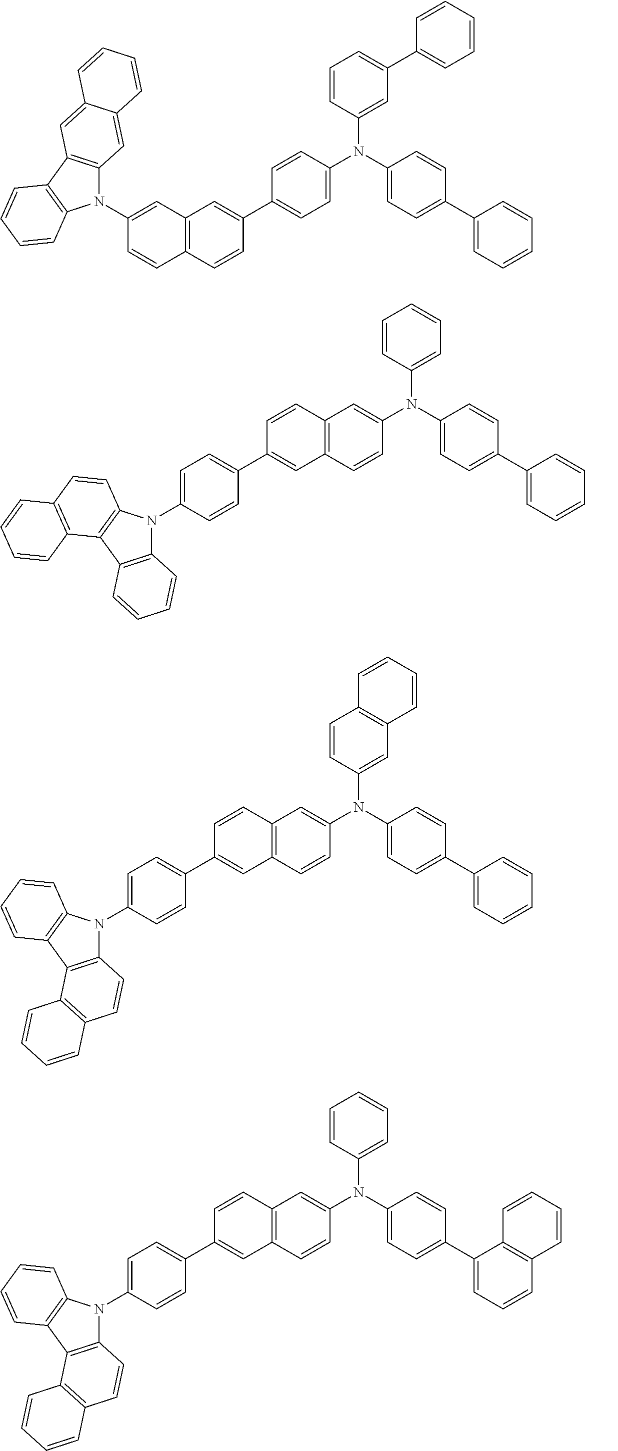 [SMILES: c1ccc(-c2ccc(N(c3ccc(-c4ccc5ccc(-n6c7ccccc7c7cc8ccccc8cc76)cc5c4)cc3)c3cccc(-c4ccccc4)c3)cc2)cc1 ; c1ccc(-c2ccc(N(c3ccccc3)c3ccc4cc(-c5ccc(-n6c7ccccc7c7c8ccccc8ccc76)cc5)ccc4c3)cc2)cc1 ; c1ccc(-c2ccc(N(c3ccc4ccccc4c3)c3ccc4cc(-c5ccc(-n6c7ccccc7c7c8ccccc8ccc76)cc5)ccc4c3)cc2)cc1 ; c1ccc(N(c2ccc(-c3cccc4ccccc34)cc2)c2ccc3cc(-c4ccc(-n5c6ccccc6c6c7ccccc7ccc65)cc4)ccc3c2)cc1] 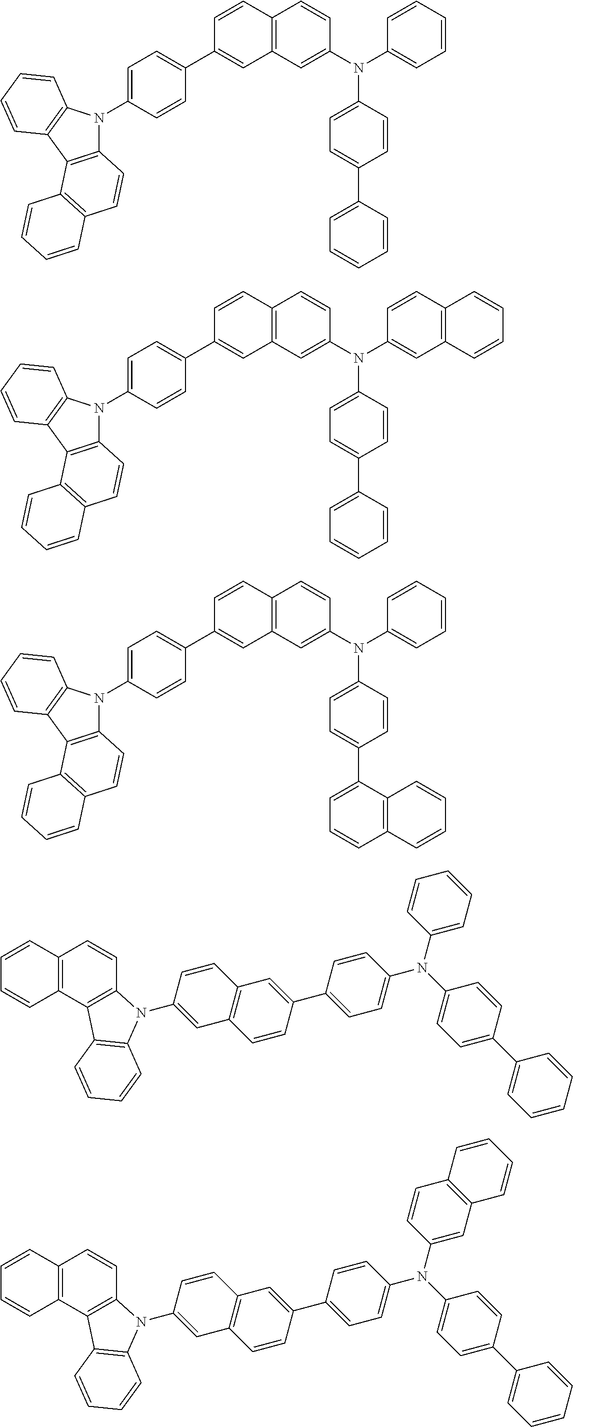 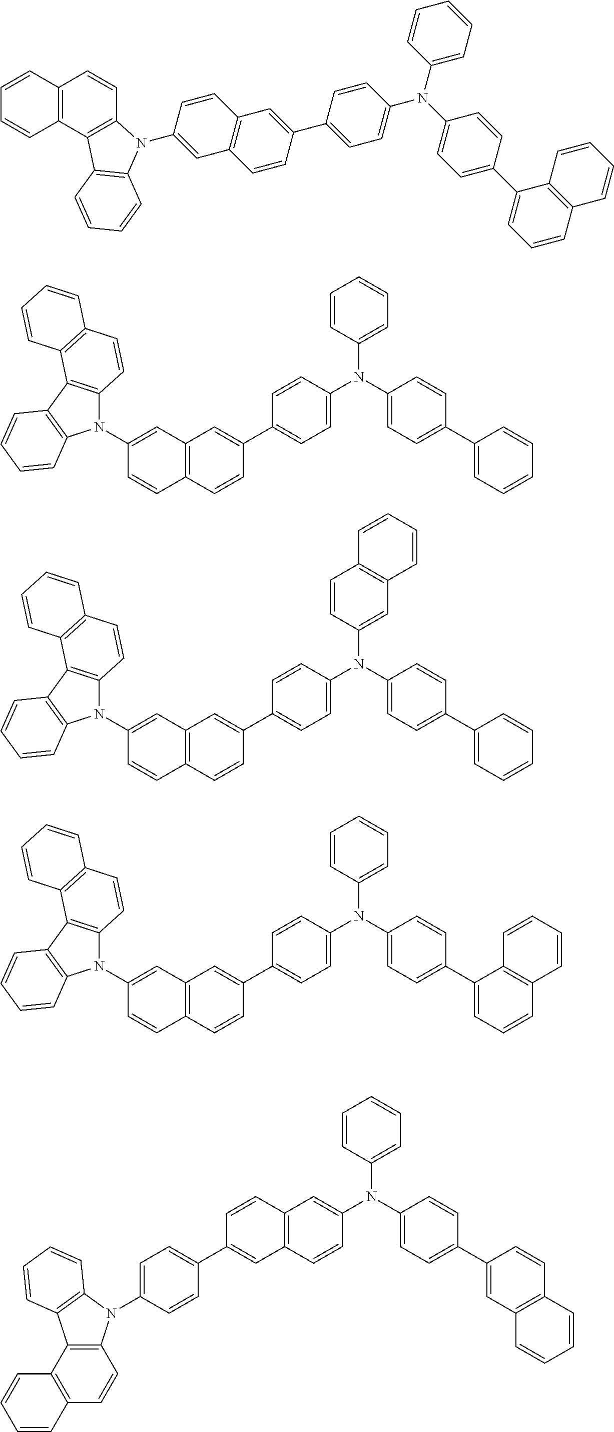 [SMILES: c1ccc(N(c2ccc(-c3ccc4cc(-n5c6ccccc6c6c7ccccc7ccc65)ccc4c3)cc2)c2ccc(-c3cccc4ccccc34)cc2)cc1 ; c1ccc(-c2ccc(N(c3ccccc3)c3ccc(-c4ccc5ccc(-n6c7ccccc7c7c8ccccc8ccc76)cc5c4)cc3)cc2)cc1 ; c1ccc(-c2ccc(N(c3ccc(-c4ccc5ccc(-n6c7ccccc7c7c8ccccc8ccc76)cc5c4)cc3)c3ccc4ccccc4c3)cc2)cc1 ; c1ccc(N(c2ccc(-c3ccc4ccc(-n5c6ccccc6c6c7ccccc7ccc65)cc4c3)cc2)c2ccc(-c3cccc4ccccc34)cc2)cc1 ; c1ccc(N(c2ccc(-c3ccc4ccccc4c3)cc2)c2ccc3cc(-c4ccc(-n5c6ccccc6c6c7ccccc7ccc65)cc4)ccc3c2)cc1] 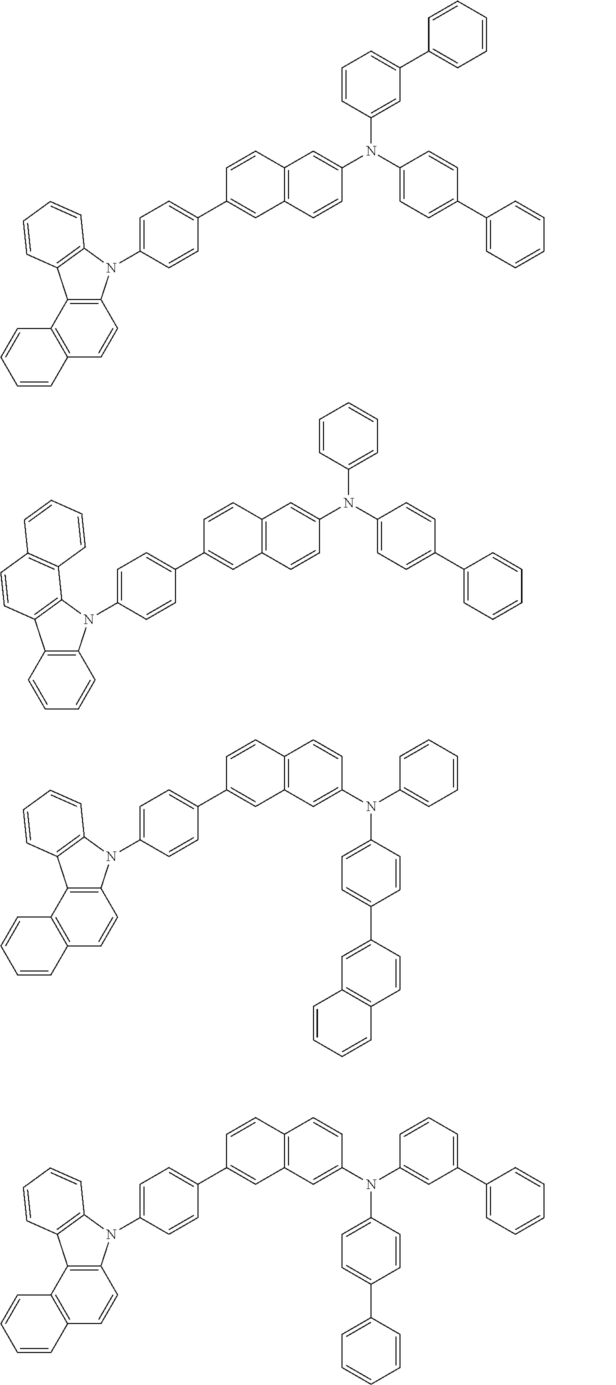 [SMILES: c1ccc(-c2ccc(N(c3cccc(-c4ccccc4)c3)c3ccc4cc(-c5ccc(-n6c7ccccc7c7c8ccccc8ccc76)cc5)ccc4c3)cc2)cc1 ; c1ccc(-c2ccc(N(c3ccccc3)c3ccc4cc(-c5ccc(-n6c7ccccc7c7ccc8ccccc8c76)cc5)ccc4c3)cc2)cc1 ; c1ccc(N(c2ccc(-c3ccc4ccccc4c3)cc2)c2ccc3ccc(-c4ccc(-n5c6ccccc6c6c7ccccc7ccc65)cc4)cc3c2)cc1 ; c1ccc(-c2ccc(N(c3cccc(-c4ccccc4)c3)c3ccc4ccc(-c5ccc(-n6c7ccccc7c7c8ccccc8ccc76)cc5)cc4c3)cc2)cc1] 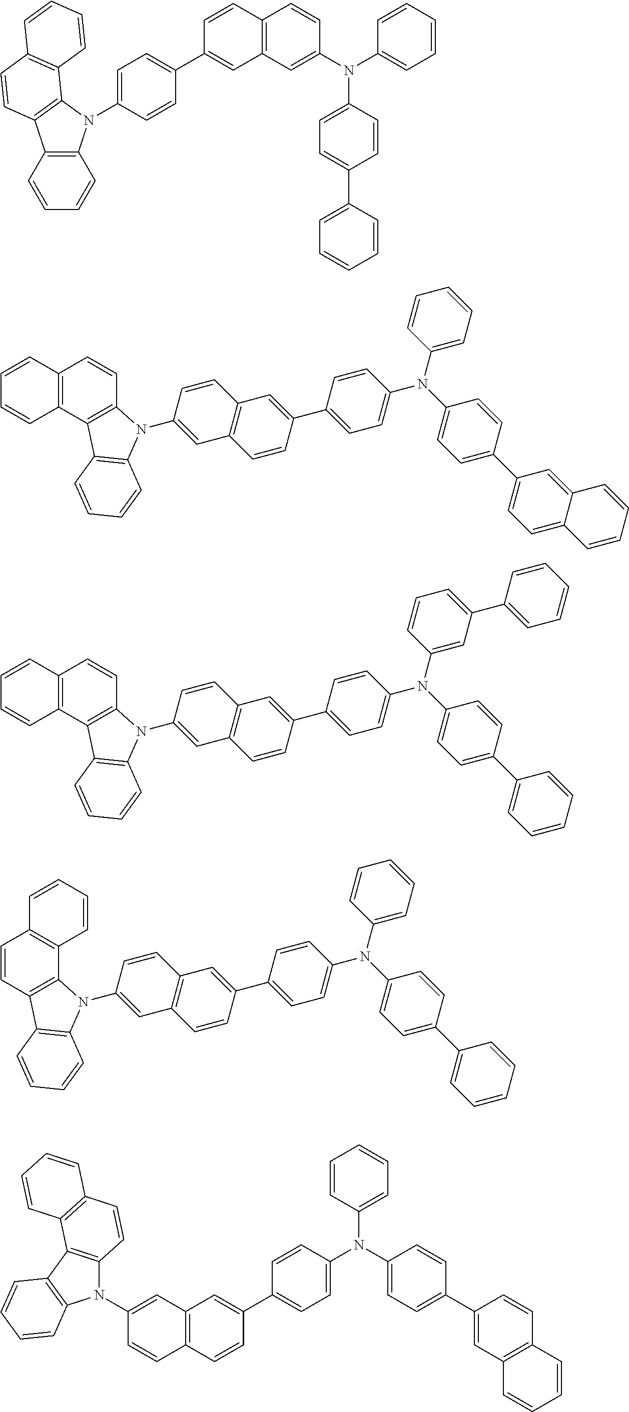 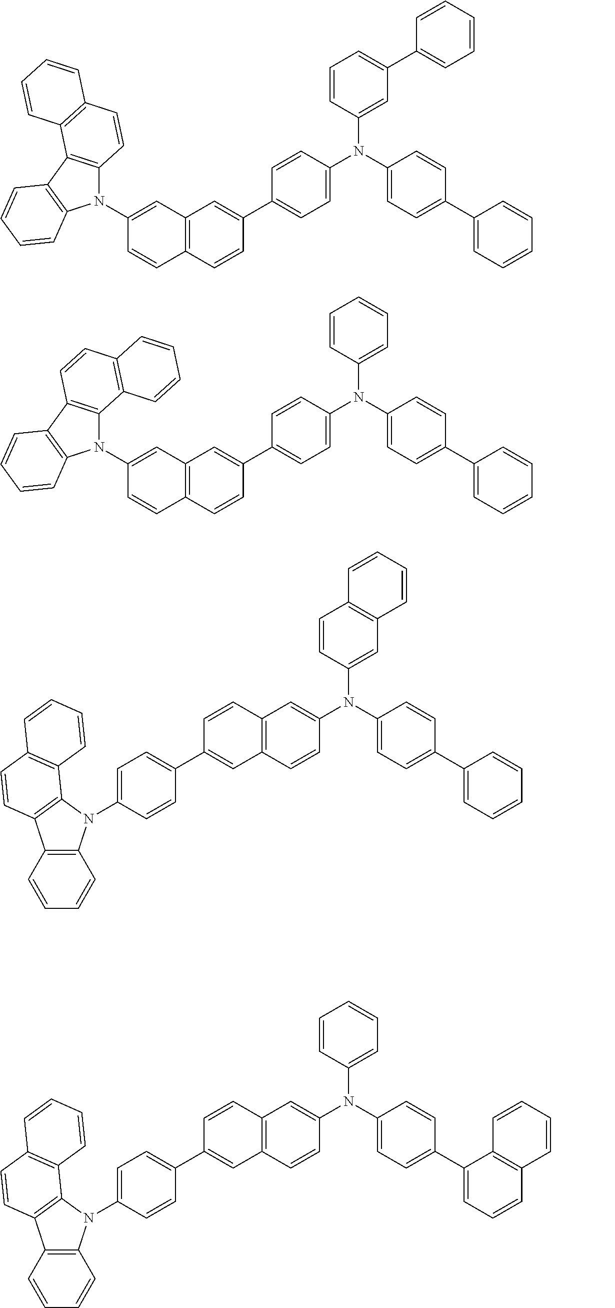 [SMILES: c1ccc(-c2ccc(N(c3ccc(-c4ccc5ccc(-n6c7ccccc7c7c8ccccc8ccc76)cc5c4)cc3)c3cccc(-c4ccccc4)c3)cc2)cc1 ; c1ccc(-c2ccc(N(c3ccccc3)c3ccc(-c4ccc5ccc(-n6c7ccccc7c7ccc8ccccc8c76)cc5c4)cc3)cc2)cc1 ; c1ccc(-c2ccc(N(c3ccc4ccccc4c3)c3ccc4cc(-c5ccc(-n6c7ccccc7c7ccc8ccccc8c76)cc5)ccc4c3)cc2)cc1 ; c1ccc(N(c2ccc(-c3cccc4ccccc34)cc2)c2ccc3cc(-c4ccc(-n5c6ccccc6c6ccc7ccccc7c65)cc4)ccc3c2)cc1] 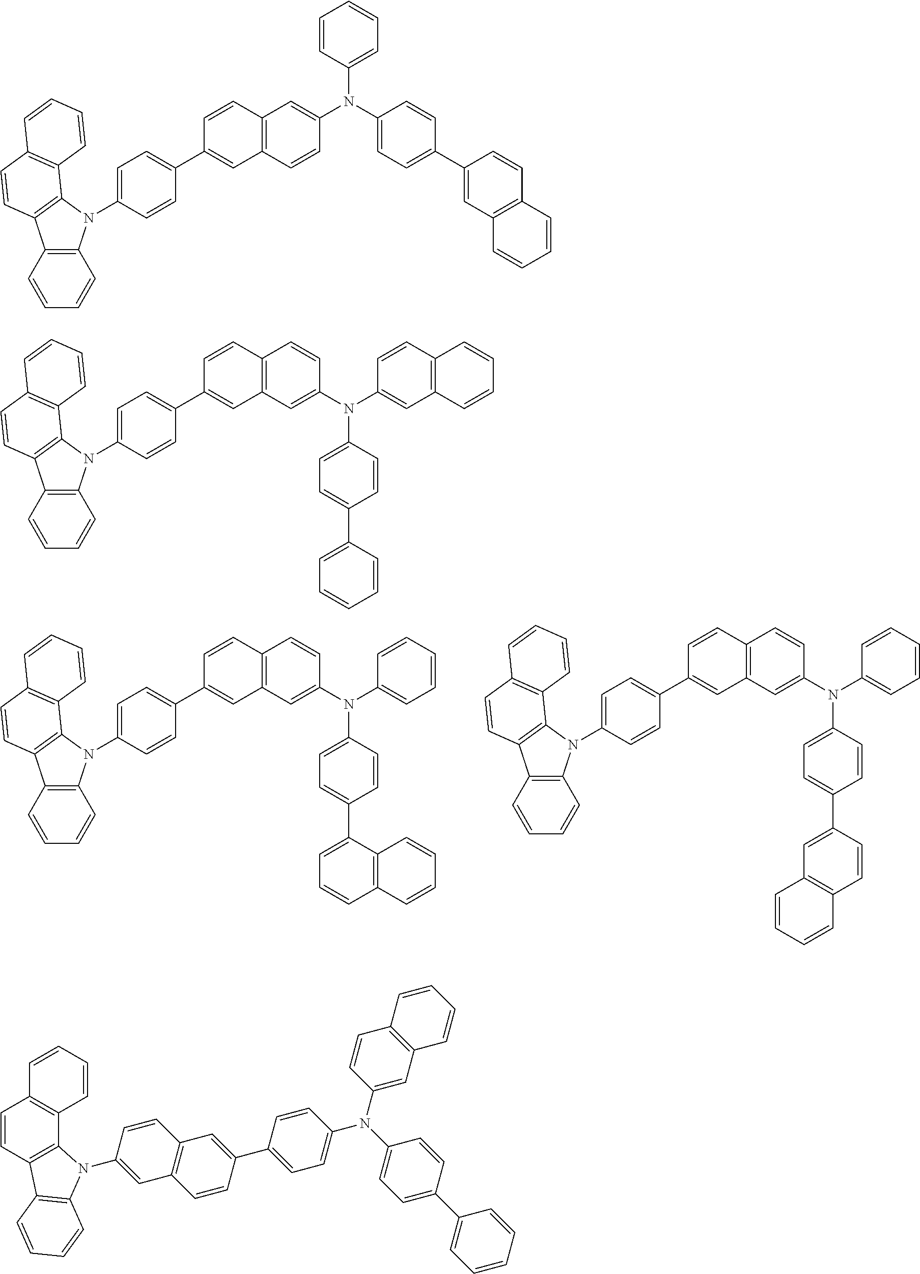 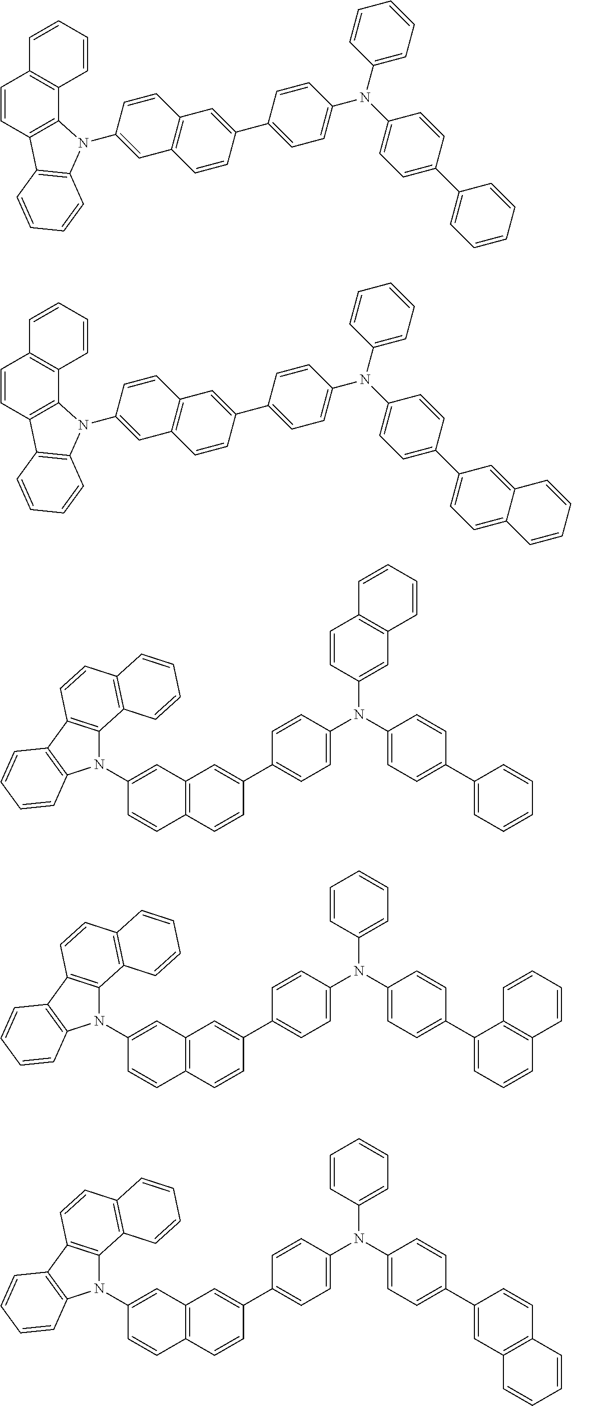 [SMILES: c1ccc(-c2ccc(N(c3ccccc3)c3ccc(-c4ccc5cc(-n6c7ccccc7c7ccc8ccccc8c76)ccc5c4)cc3)cc2)cc1 ; c1ccc(N(c2ccc(-c3ccc4ccccc4c3)cc2)c2ccc(-c3ccc4cc(-n5c6ccccc6c6ccc7ccccc7c65)ccc4c3)cc2)cc1 ; c1ccc(-c2ccc(N(c3ccc(-c4ccc5ccc(-n6c7ccccc7c7ccc8ccccc8c76)cc5c4)cc3)c3ccc4ccccc4c3)cc2)cc1 ; c1ccc(N(c2ccc(-c3ccc4ccc(-n5c6ccccc6c6ccc7ccccc7c65)cc4c3)cc2)c2ccc(-c3cccc4ccccc34)cc2)cc1 ; c1ccc(N(c2ccc(-c3ccc4ccccc4c3)cc2)c2ccc(-c3ccc4ccc(-n5c6ccccc6c6ccc7ccccc7c65)cc4c3)cc2)cc1] 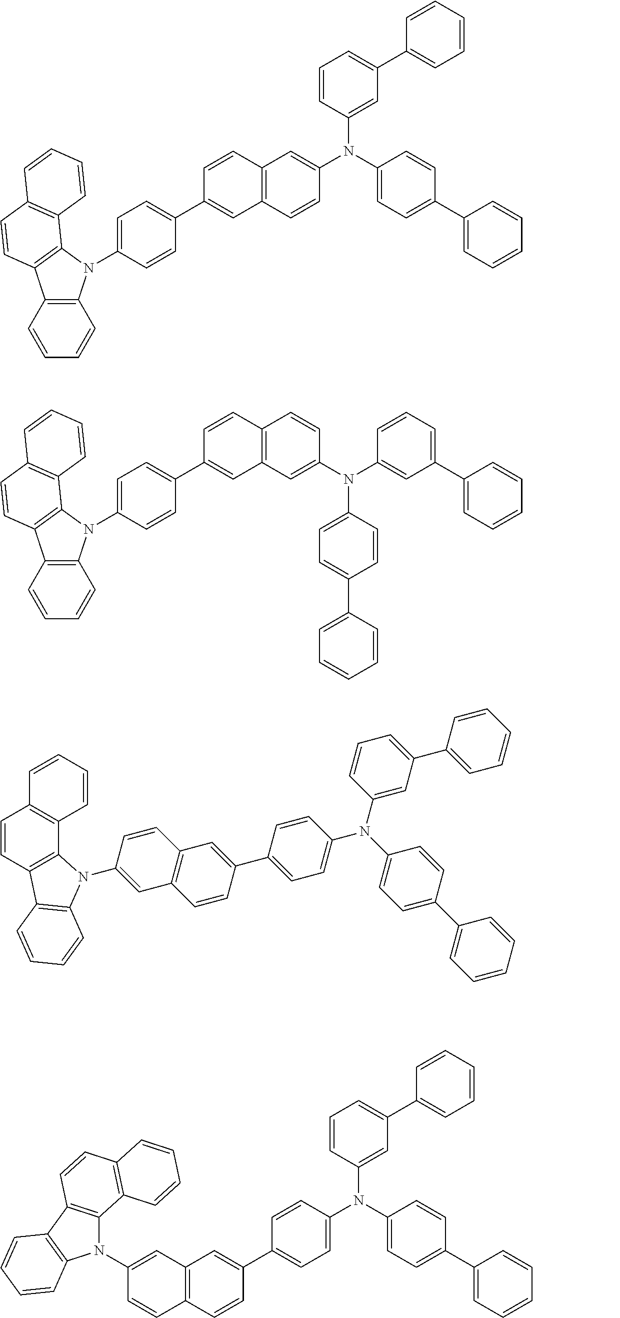 [SMILES: c1ccc(-c2ccc(N(c3cccc(-c4ccccc4)c3)c3ccc4cc(-c5ccc(-n6c7ccccc7c7ccc8ccccc8c76)cc5)ccc4c3)cc2)cc1 ; c1ccc(-c2ccc(N(c3cccc(-c4ccccc4)c3)c3ccc4ccc(-c5ccc(-n6c7ccccc7c7ccc8ccccc8c76)cc5)cc4c3)cc2)cc1 ; c1ccc(-c2ccc(N(c3ccc(-c4ccc5cc(-n6c7ccccc7c7ccc8ccccc8c76)ccc5c4)cc3)c3cccc(-c4ccccc4)c3)cc2)cc1 ; c1ccc(-c2ccc(N(c3ccc(-c4ccc5ccc(-n6c7ccccc7c7ccc8ccccc8c76)cc5c4)cc3)c3cccc(-c4ccccc4)c3)cc2)cc1] 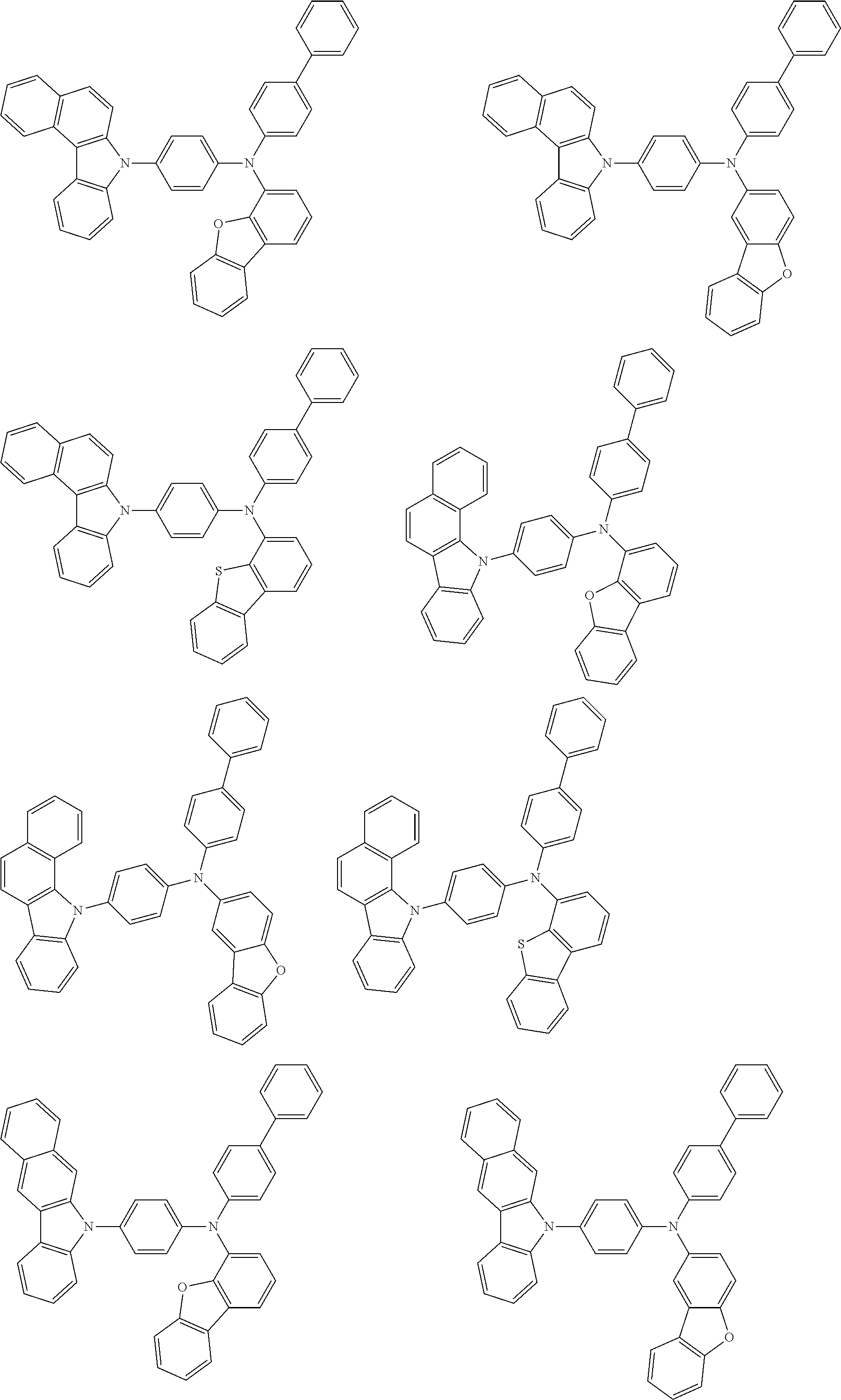 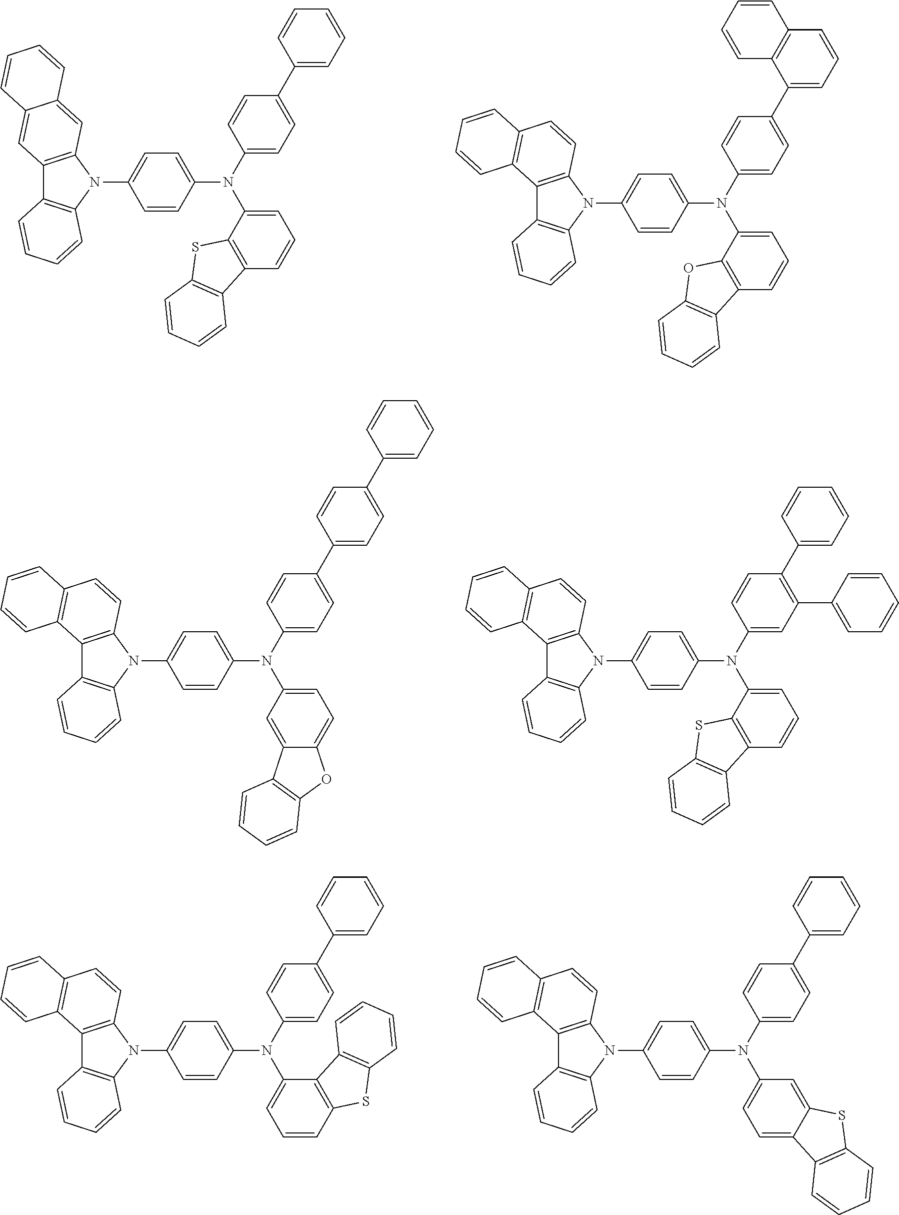 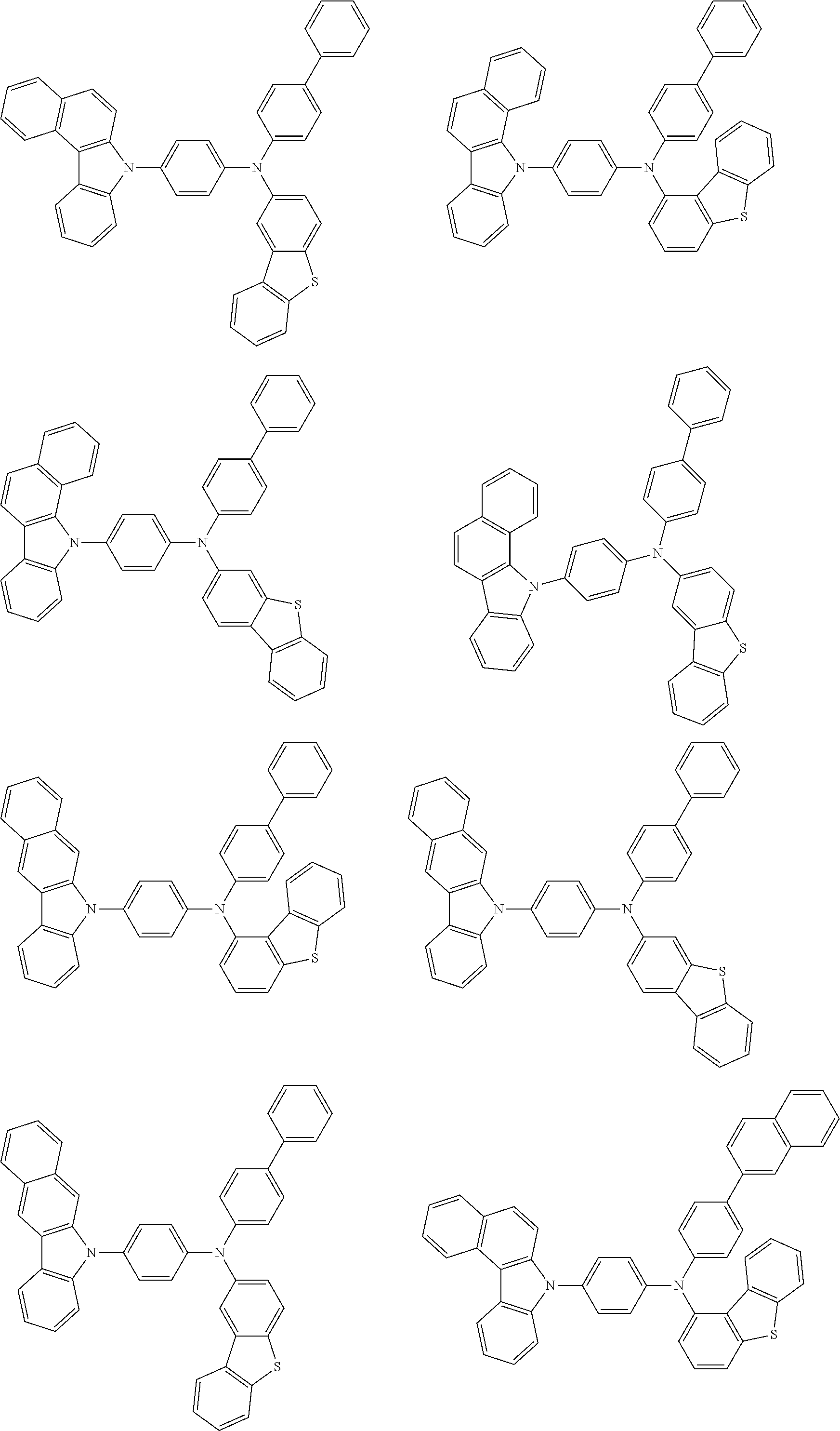 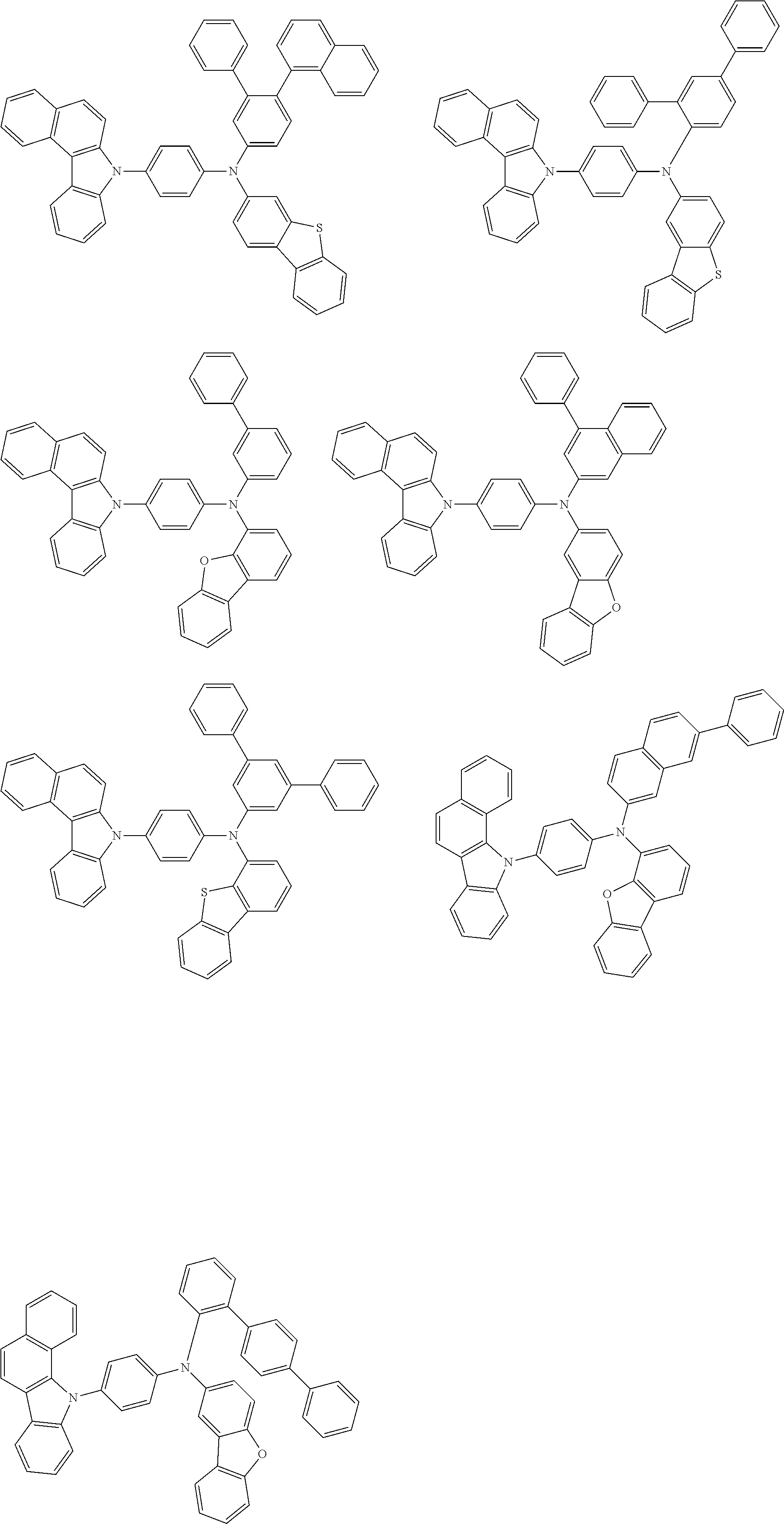 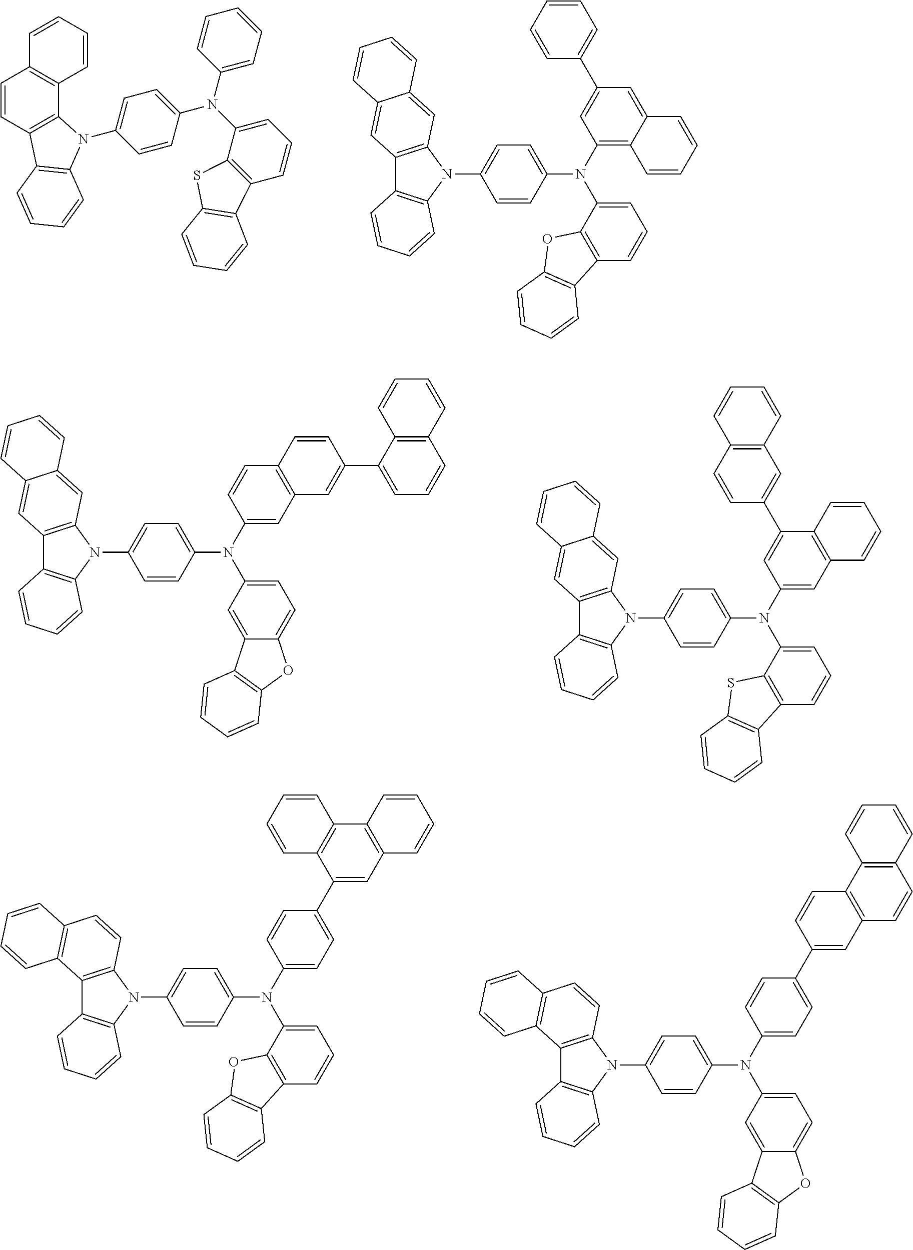 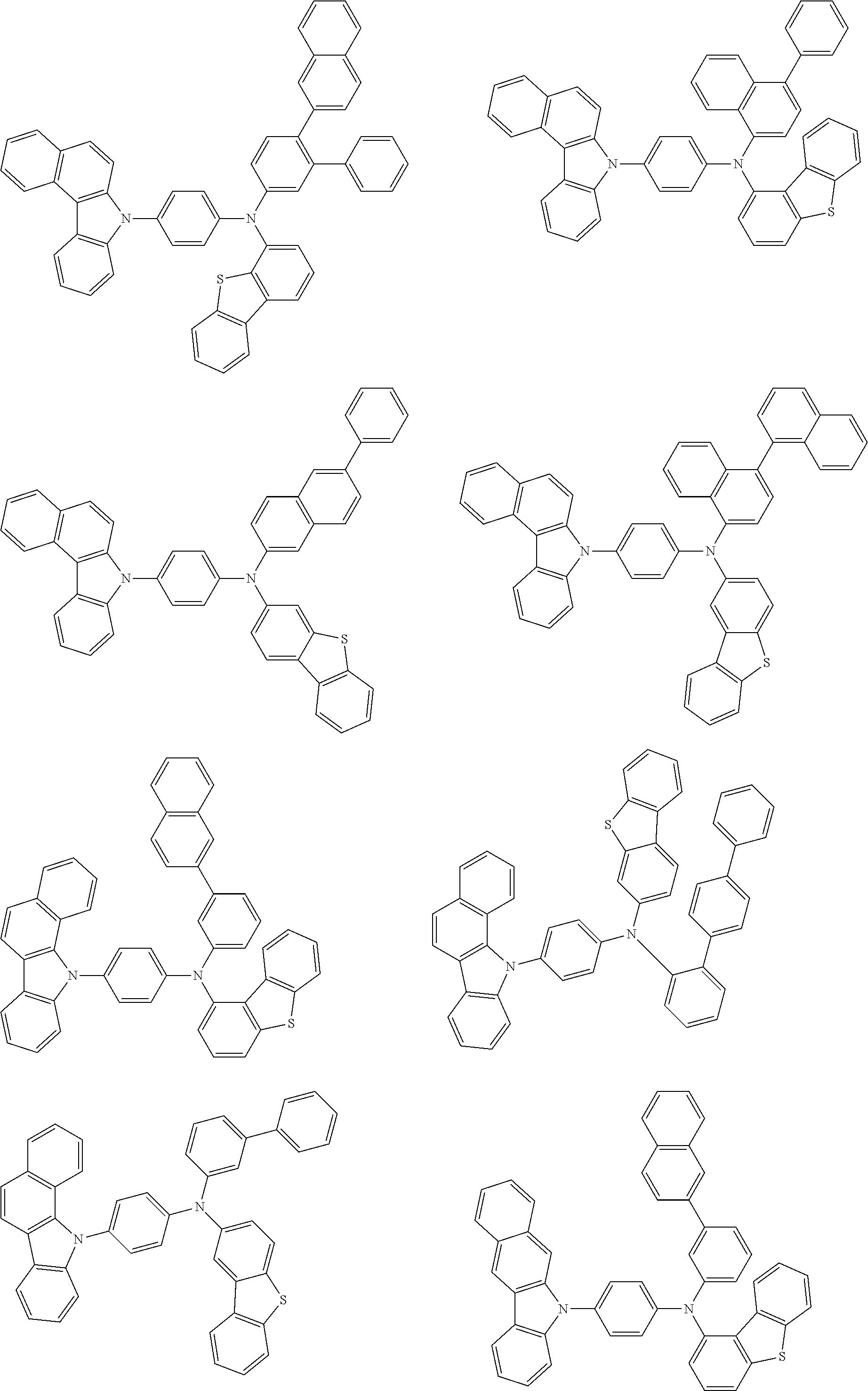 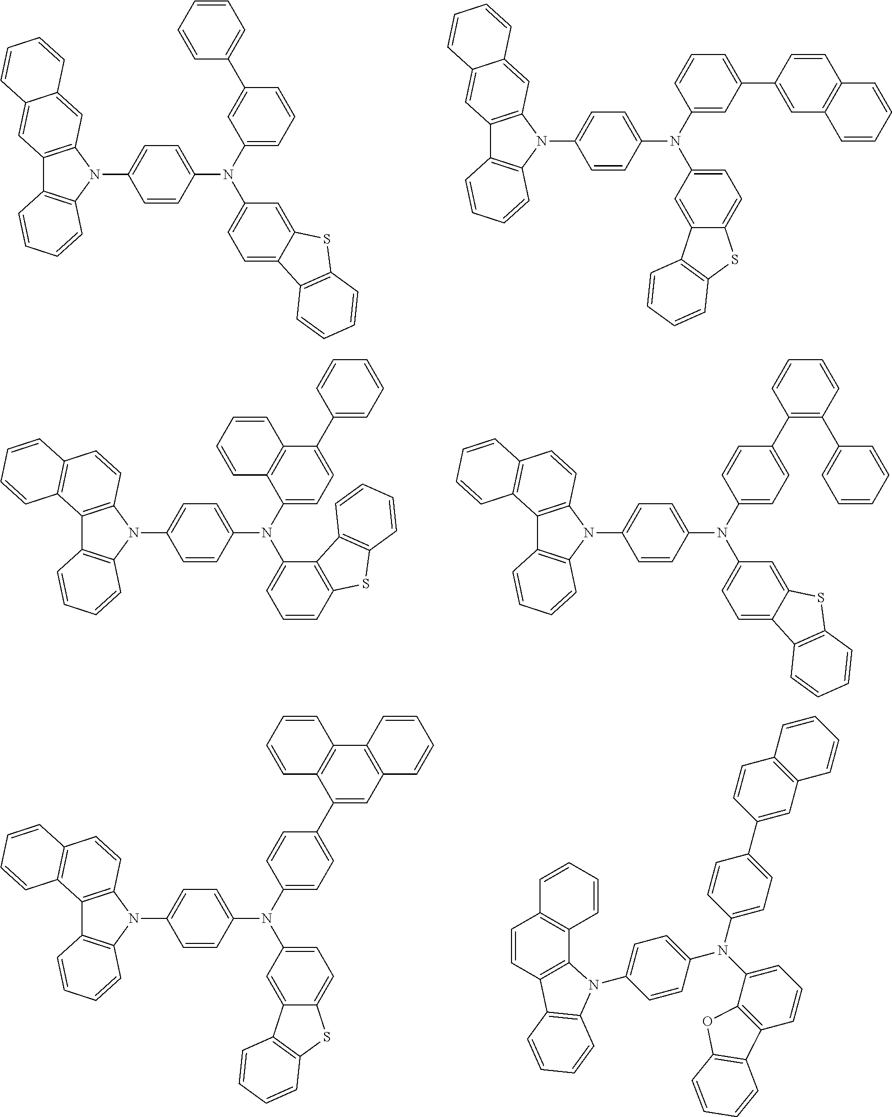 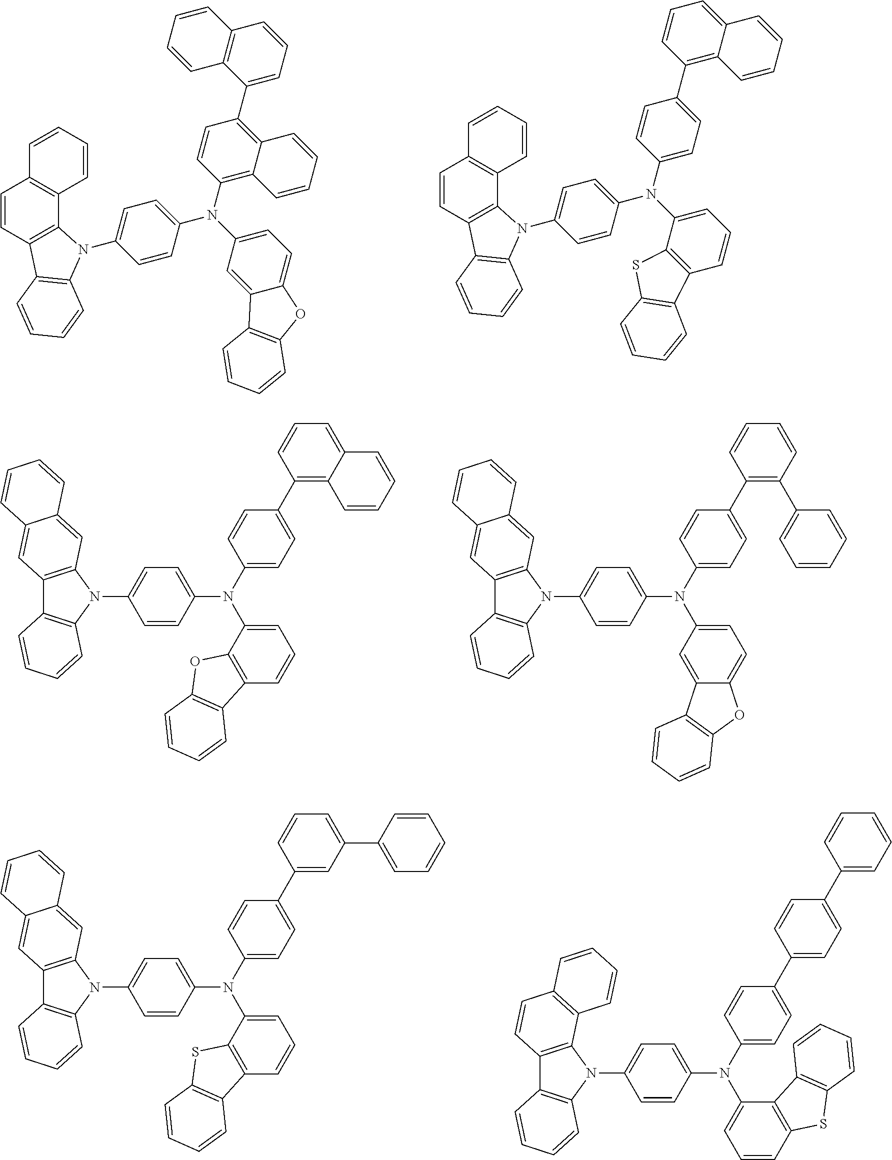 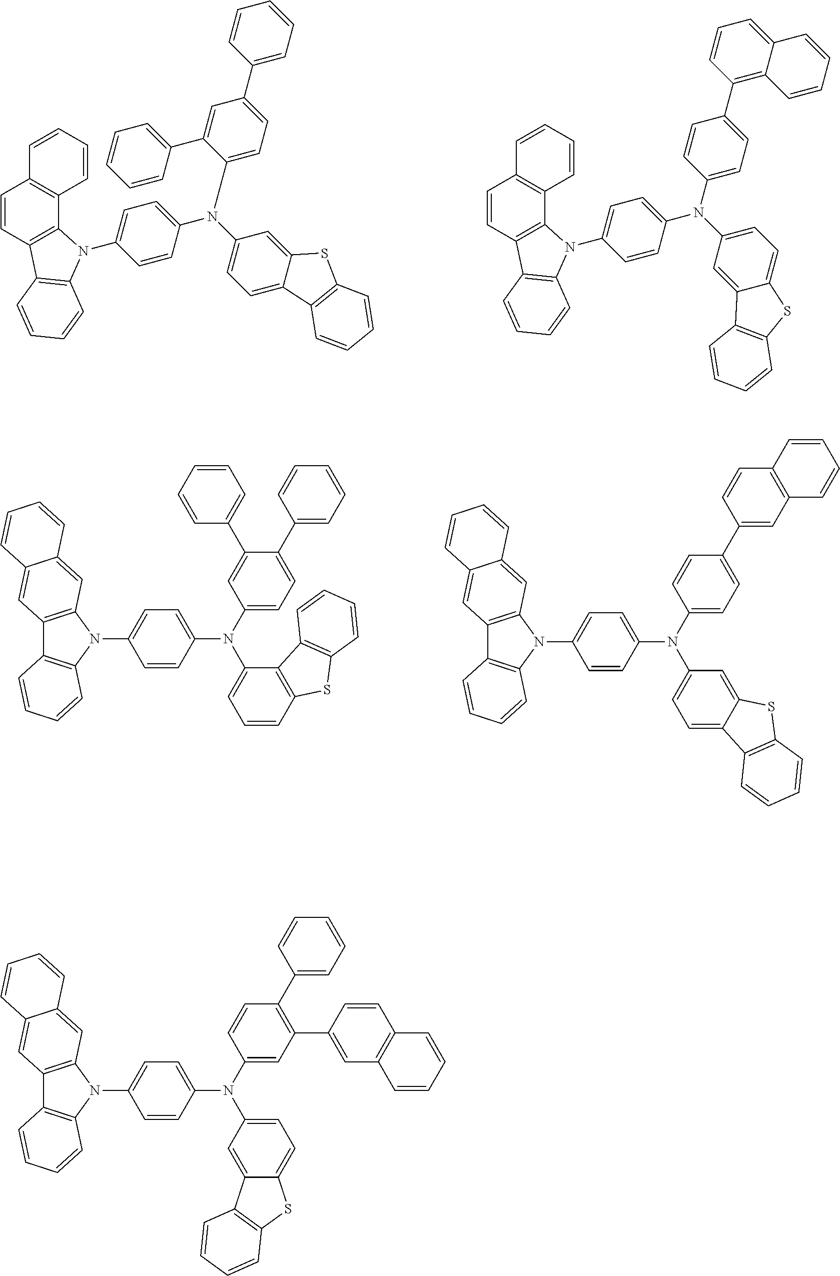 [SMILES: c1ccc(-c2ccc(N(c3ccc(-n4c5ccccc5c5ccc6ccccc6c54)cc3)c3ccc4c(c3)sc3ccccc34)c(-c3ccccc3)c2)cc1 ; c1ccc2c(-c3ccc(N(c4ccc(-n5c6ccccc6c6ccc7ccccc7c65)cc4)c4ccc5sc6ccccc6c5c4)cc3)cccc2c1 ; c1ccc(-c2ccc(N(c3ccc(-n4c5ccccc5c5cc6ccccc6cc54)cc3)c3cccc4sc5ccccc5c34)cc2-c2ccccc2)cc1 ; c1ccc2cc(-c3ccc(N(c4ccc(-n5c6ccccc6c6cc7ccccc7cc65)cc4)c4ccc5c(c4)sc4ccccc45)cc3)ccc2c1 ; c1ccc(-c2ccc(N(c3ccc(-n4c5ccccc5c5cc6ccccc6cc54)cc3)c3ccc4sc5ccccc5c4c3)cc2-c2ccc3ccccc3c2)cc1] 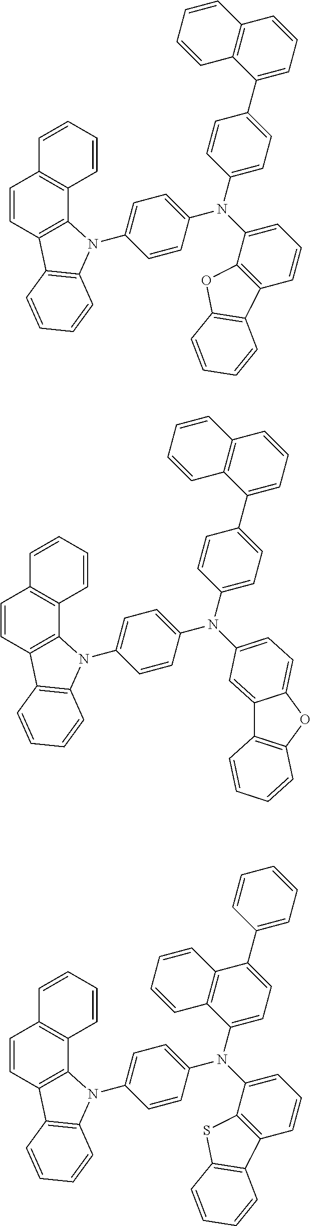 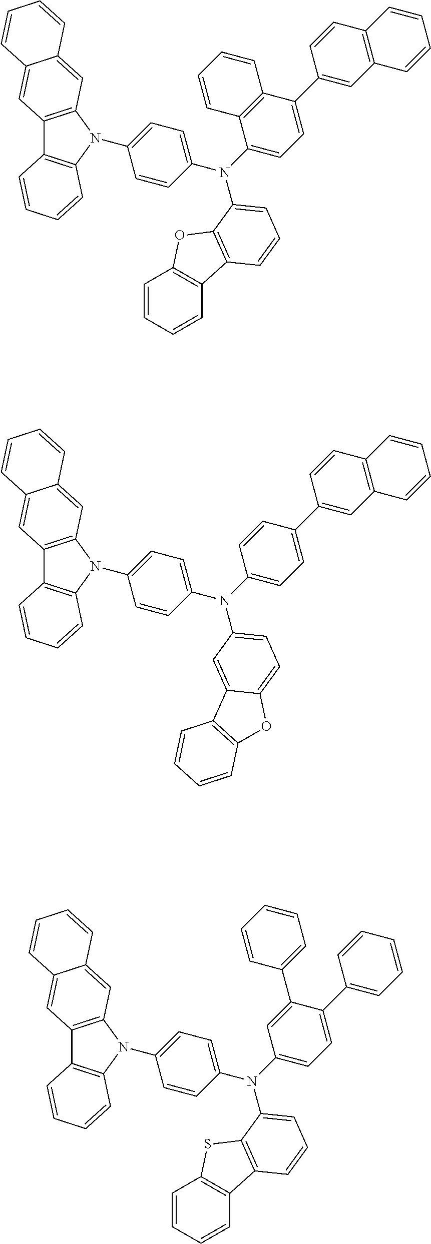 [SMILES: c1ccc2cc(-c3ccc(N(c4ccc(-n5c6ccccc6c6cc7ccccc7cc65)cc4)c4cccc5c4oc4ccccc45)c4ccccc34)ccc2c1 ; c1ccc2cc(-c3ccc(N(c4ccc(-n5c6ccccc6c6cc7ccccc7cc65)cc4)c4ccc5oc6ccccc6c5c4)cc3)ccc2c1 ; c1ccc(-c2ccc(N(c3ccc(-n4c5ccccc5c5cc6ccccc6cc54)cc3)c3cccc4c3sc3ccccc34)cc2-c2ccccc2)cc1] 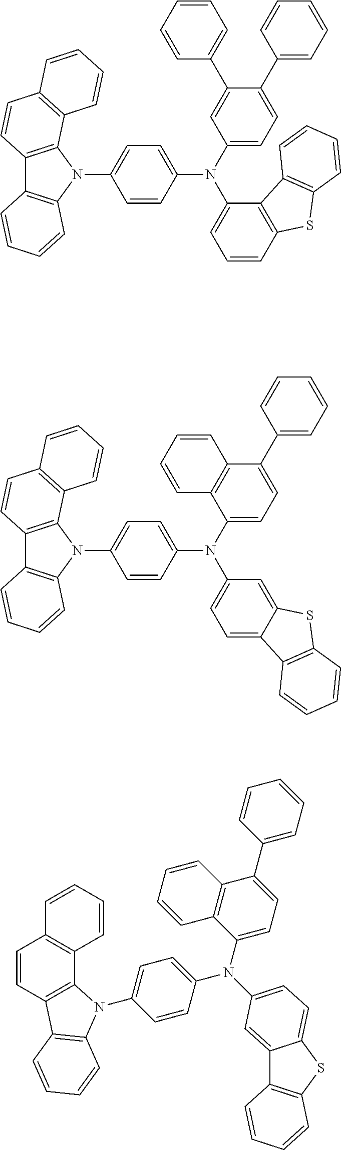 [SMILES: c1ccc(-c2ccc(N(c3ccc(-n4c5ccccc5c5ccc6ccccc6c54)cc3)c3cccc4sc5ccccc5c34)cc2-c2ccccc2)cc1 ; c1ccc(-c2ccc(N(c3ccc(-n4c5ccccc5c5ccc6ccccc6c54)cc3)c3ccc4c(c3)sc3ccccc34)c3ccccc23)cc1 ; c1ccc(-c2ccc(N(c3ccc(-n4c5ccccc5c5ccc6ccccc6c54)cc3)c3ccc4sc5ccccc5c4c3)c3ccccc23)cc1] 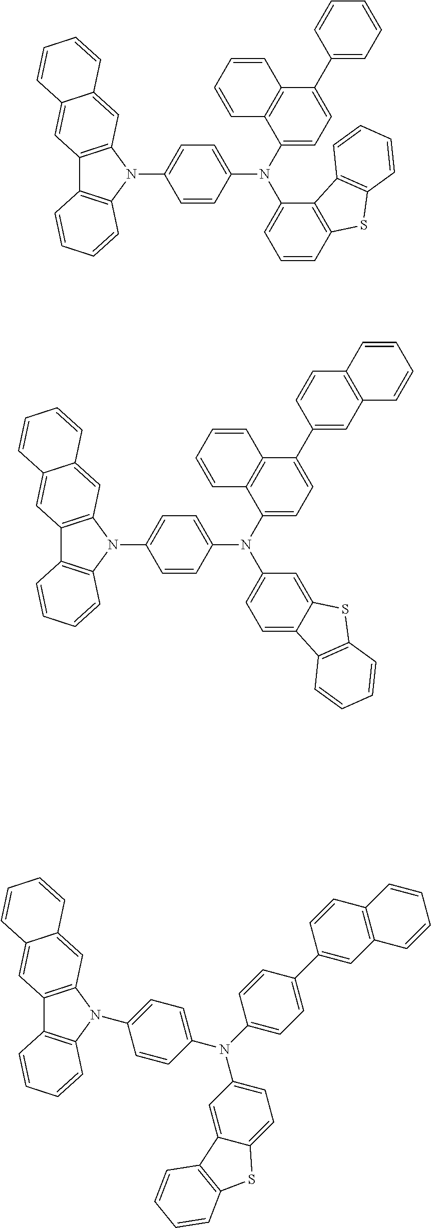 [SMILES: c1ccc(-c2ccc(N(c3ccc(-n4c5ccccc5c5cc6ccccc6cc54)cc3)c3cccc4sc5ccccc5c34)c3ccccc23)cc1 ; c1ccc2cc(-c3ccc(N(c4ccc(-n5c6ccccc6c6cc7ccccc7cc65)cc4)c4ccc5c(c4)sc4ccccc45)c4ccccc34)ccc2c1 ; c1ccc2cc(-c3ccc(N(c4ccc(-n5c6ccccc6c6cc7ccccc7cc65)cc4)c4ccc5sc6ccccc6c5c4)cc3)ccc2c1] 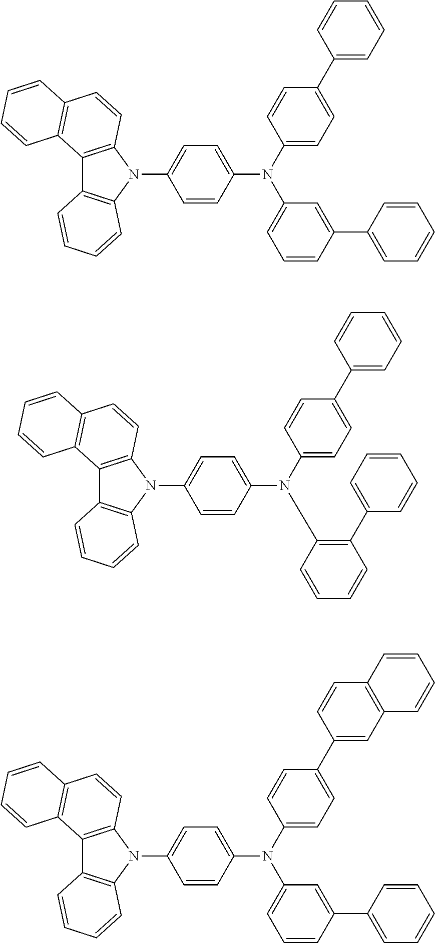 [SMILES: c1ccc(-c2ccc(N(c3ccc(-n4c5ccccc5c5c6ccccc6ccc54)cc3)c3cccc(-c4ccccc4)c3)cc2)cc1 ; c1ccc(-c2ccc(N(c3ccc(-n4c5ccccc5c5c6ccccc6ccc54)cc3)c3ccccc3-c3ccccc3)cc2)cc1 ; c1ccc(-c2cccc(N(c3ccc(-c4ccc5ccccc5c4)cc3)c3ccc(-n4c5ccccc5c5c6ccccc6ccc54)cc3)c2)cc1] 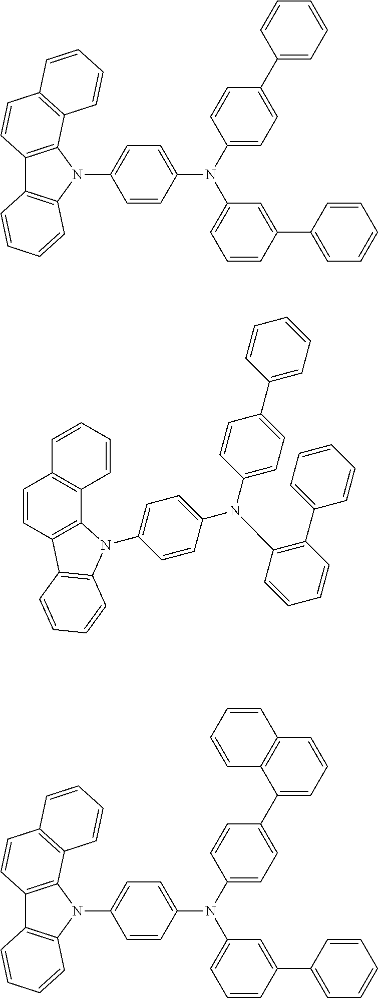 [SMILES: c1ccc(-c2ccc(N(c3ccc(-n4c5ccccc5c5ccc6ccccc6c54)cc3)c3cccc(-c4ccccc4)c3)cc2)cc1 ; c1ccc(-c2ccc(N(c3ccc(-n4c5ccccc5c5ccc6ccccc6c54)cc3)c3ccccc3-c3ccccc3)cc2)cc1 ; c1ccc(-c2cccc(N(c3ccc(-c4cccc5ccccc45)cc3)c3ccc(-n4c5ccccc5c5ccc6ccccc6c54)cc3)c2)cc1] 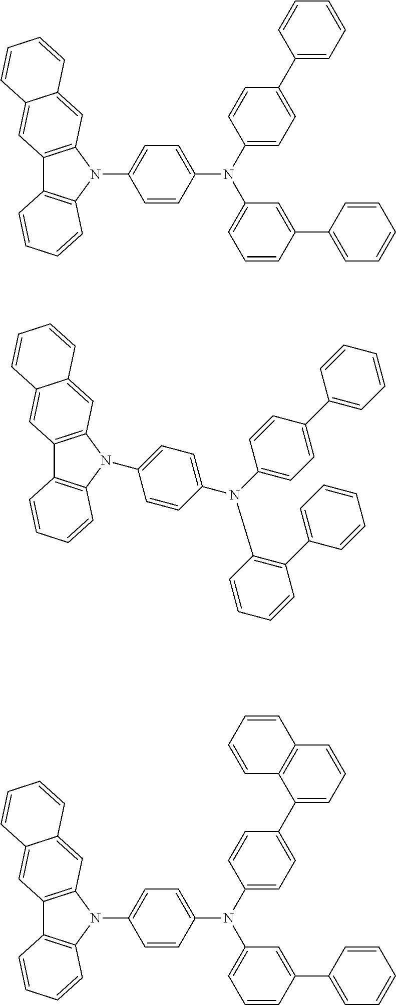 [SMILES: c1ccc(-c2ccc(N(c3ccc(-n4c5ccccc5c5cc6ccccc6cc54)cc3)c3cccc(-c4ccccc4)c3)cc2)cc1 ; c1ccc(-c2ccc(N(c3ccc(-n4c5ccccc5c5cc6ccccc6cc54)cc3)c3ccccc3-c3ccccc3)cc2)cc1 ; c1ccc(-c2cccc(N(c3ccc(-c4cccc5ccccc45)cc3)c3ccc(-n4c5ccccc5c5cc6ccccc6cc54)cc3)c2)cc1] 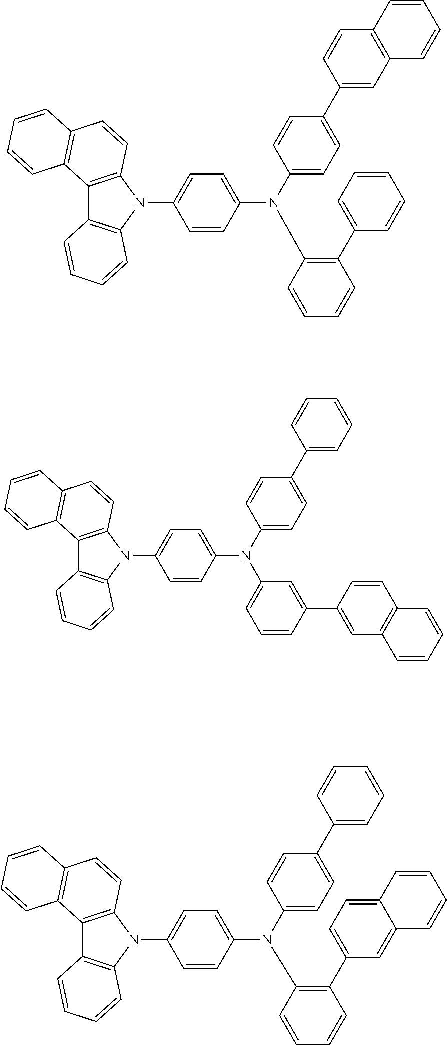 [SMILES: c1ccc(-c2ccccc2N(c2ccc(-c3ccc4ccccc4c3)cc2)c2ccc(-n3c4ccccc4c4c5ccccc5ccc43)cc2)cc1 ; c1ccc(-c2ccc(N(c3ccc(-n4c5ccccc5c5c6ccccc6ccc54)cc3)c3cccc(-c4ccc5ccccc5c4)c3)cc2)cc1 ; c1ccc(-c2ccc(N(c3ccc(-n4c5ccccc5c5c6ccccc6ccc54)cc3)c3ccccc3-c3ccc4ccccc4c3)cc2)cc1] 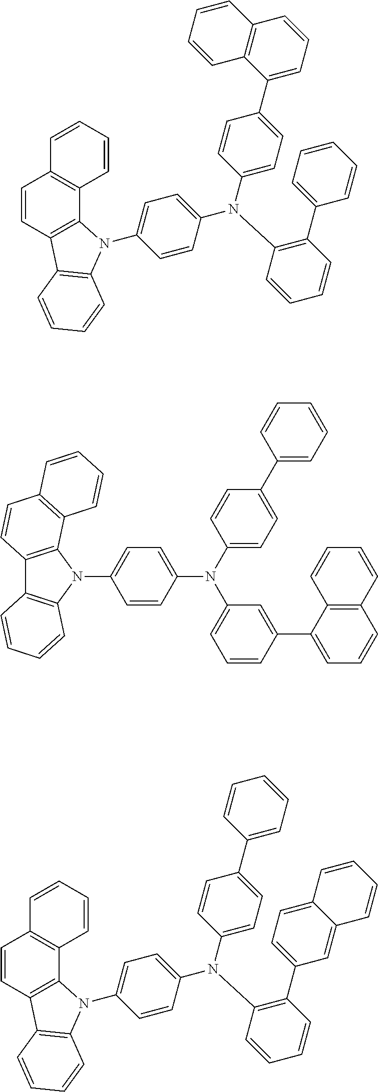 [SMILES: c1ccc(-c2ccccc2N(c2ccc(-c3cccc4ccccc34)cc2)c2ccc(-n3c4ccccc4c4ccc5ccccc5c43)cc2)cc1 ; c1ccc(-c2ccc(N(c3ccc(-n4c5ccccc5c5ccc6ccccc6c54)cc3)c3cccc(-c4cccc5ccccc45)c3)cc2)cc1 ; c1ccc(-c2ccc(N(c3ccc(-n4c5ccccc5c5ccc6ccccc6c54)cc3)c3ccccc3-c3ccc4ccccc4c3)cc2)cc1] 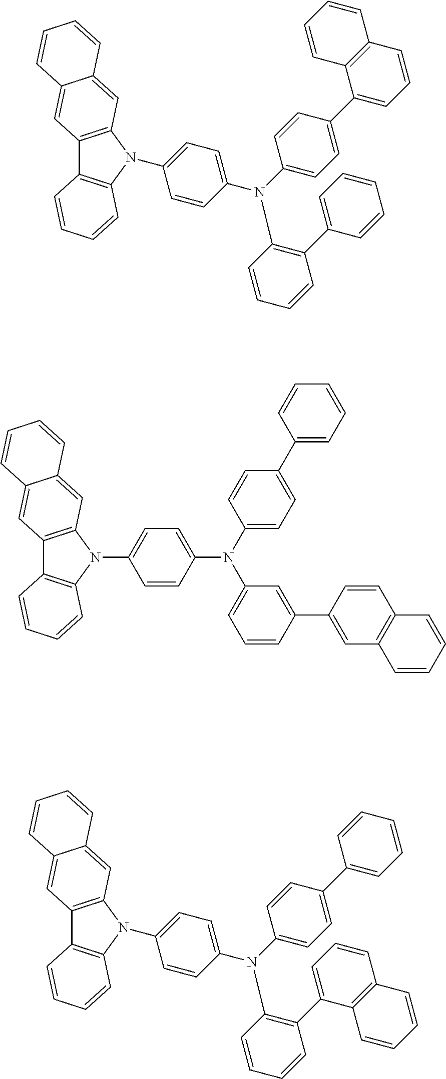 [SMILES: c1ccc(-c2ccccc2N(c2ccc(-c3cccc4ccccc34)cc2)c2ccc(-n3c4ccccc4c4cc5ccccc5cc43)cc2)cc1 ; c1ccc(-c2ccc(N(c3ccc(-n4c5ccccc5c5cc6ccccc6cc54)cc3)c3cccc(-c4ccc5ccccc5c4)c3)cc2)cc1 ; c1ccc(-c2ccc(N(c3ccc(-n4c5ccccc5c5cc6ccccc6cc54)cc3)c3ccccc3-c3cccc4ccccc34)cc2)cc1] 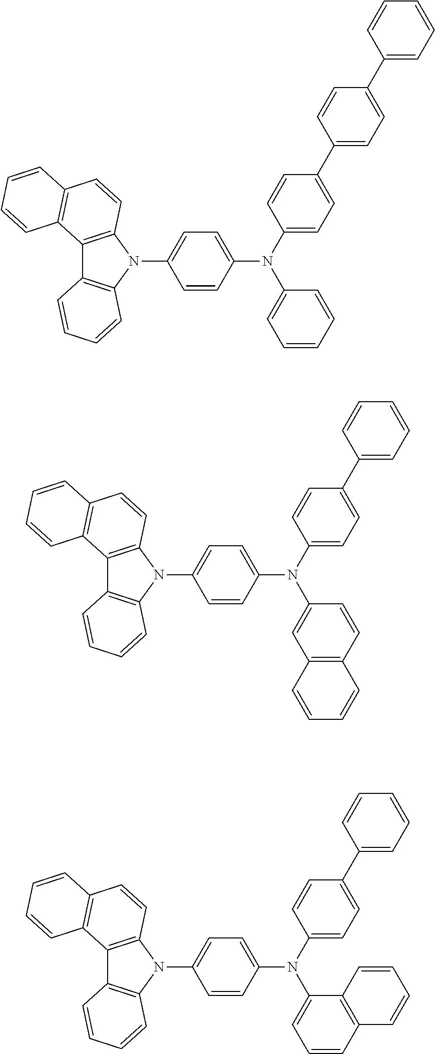 [SMILES: c1ccc(-c2ccc(-c3ccc(N(c4ccccc4)c4ccc(-n5c6ccccc6c6c7ccccc7ccc65)cc4)cc3)cc2)cc1 ; c1ccc(-c2ccc(N(c3ccc(-n4c5ccccc5c5c6ccccc6ccc54)cc3)c3ccc4ccccc4c3)cc2)cc1 ; c1ccc(-c2ccc(N(c3ccc(-n4c5ccccc5c5c6ccccc6ccc54)cc3)c3cccc4ccccc34)cc2)cc1] 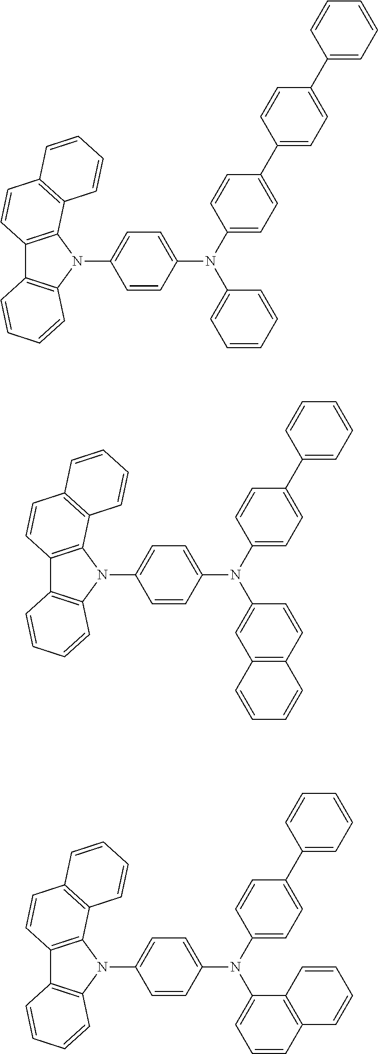 [SMILES: c1ccc(-c2ccc(-c3ccc(N(c4ccccc4)c4ccc(-n5c6ccccc6c6ccc7ccccc7c65)cc4)cc3)cc2)cc1 ; c1ccc(-c2ccc(N(c3ccc(-n4c5ccccc5c5ccc6ccccc6c54)cc3)c3ccc4ccccc4c3)cc2)cc1 ; c1ccc(-c2ccc(N(c3ccc(-n4c5ccccc5c5ccc6ccccc6c54)cc3)c3cccc4ccccc34)cc2)cc1] 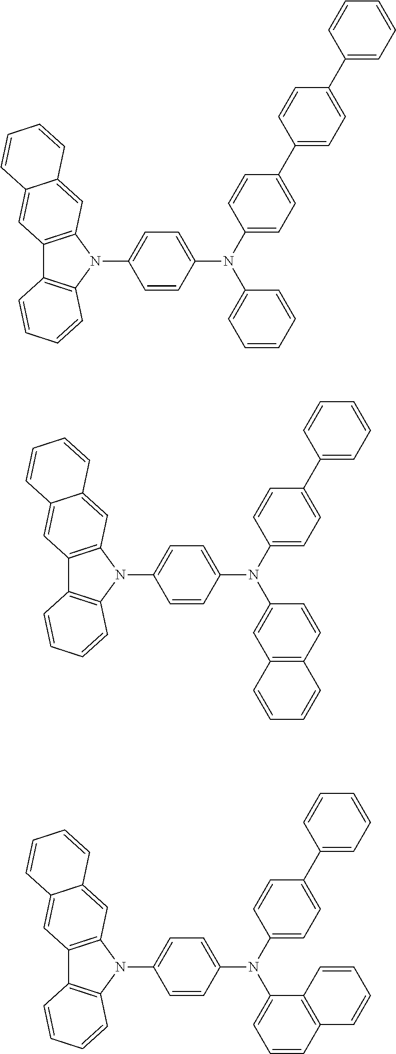 [SMILES: c1ccc(-c2ccc(-c3ccc(N(c4ccccc4)c4ccc(-n5c6ccccc6c6cc7ccccc7cc65)cc4)cc3)cc2)cc1 ; c1ccc(-c2ccc(N(c3ccc(-n4c5ccccc5c5cc6ccccc6cc54)cc3)c3ccc4ccccc4c3)cc2)cc1 ; c1ccc(-c2ccc(N(c3ccc(-n4c5ccccc5c5cc6ccccc6cc54)cc3)c3cccc4ccccc34)cc2)cc1] 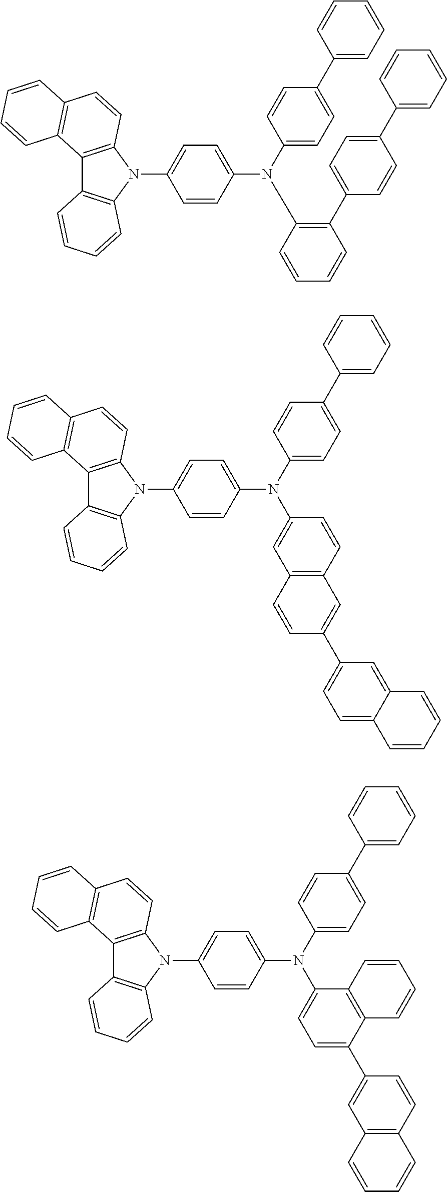 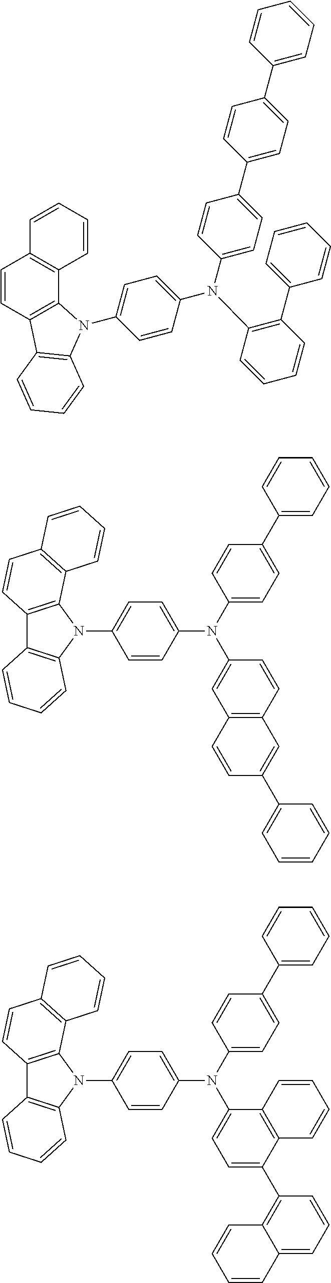 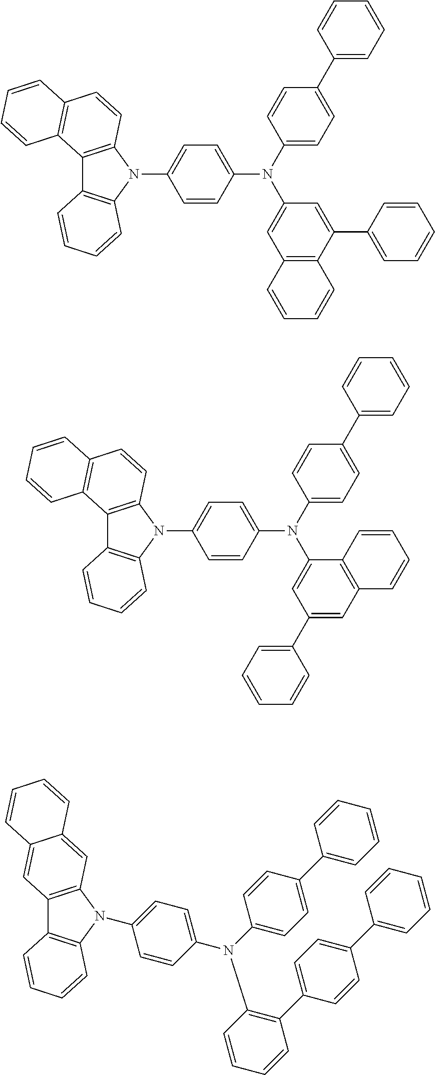 [SMILES: c1ccc(-c2ccc(N(c3ccc(-n4c5ccccc5c5c6ccccc6ccc54)cc3)c3cc(-c4ccccc4)c4ccccc4c3)cc2)cc1 ; c1ccc(-c2ccc(N(c3ccc(-n4c5ccccc5c5c6ccccc6ccc54)cc3)c3cc(-c4ccccc4)cc4ccccc34)cc2)cc1 ; c1ccc(-c2ccc(-c3ccccc3N(c3ccc(-c4ccccc4)cc3)c3ccc(-n4c5ccccc5c5cc6ccccc6cc54)cc3)cc2)cc1] 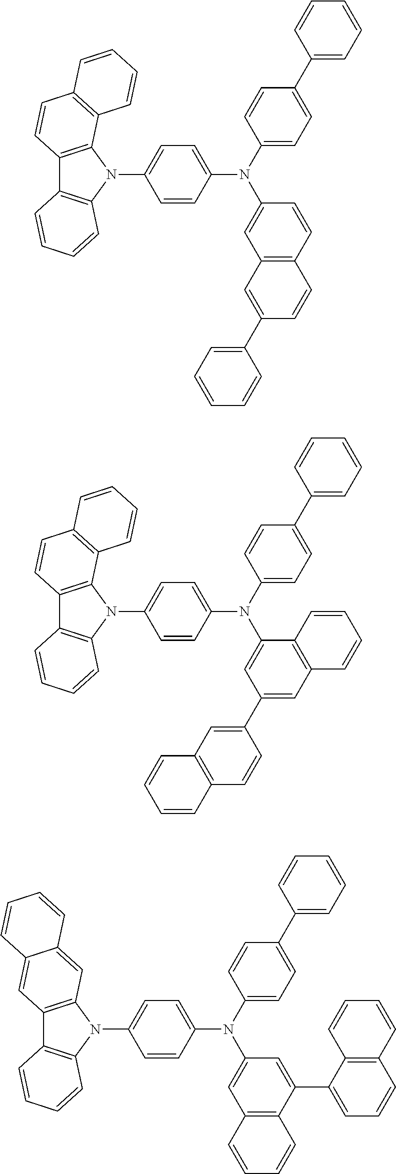 [SMILES: c1ccc(-c2ccc(N(c3ccc(-n4c5ccccc5c5ccc6ccccc6c54)cc3)c3ccc4ccc(-c5ccccc5)cc4c3)cc2)cc1 ; c1ccc(-c2ccc(N(c3ccc(-n4c5ccccc5c5ccc6ccccc6c54)cc3)c3cc(-c4ccc5ccccc5c4)cc4ccccc34)cc2)cc1 ; c1ccc(-c2ccc(N(c3ccc(-n4c5ccccc5c5cc6ccccc6cc54)cc3)c3cc(-c4cccc5ccccc45)c4ccccc4c3)cc2)cc1] 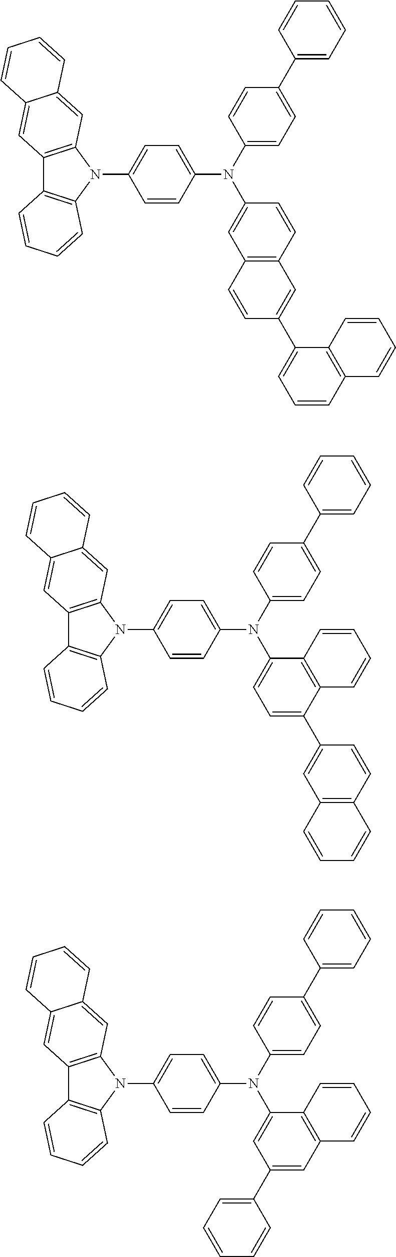 [SMILES: c1ccc(-c2ccc(N(c3ccc(-n4c5ccccc5c5cc6ccccc6cc54)cc3)c3ccc4cc(-c5cccc6ccccc56)ccc4c3)cc2)cc1 ; c1ccc(-c2ccc(N(c3ccc(-n4c5ccccc5c5cc6ccccc6cc54)cc3)c3ccc(-c4ccc5ccccc5c4)c4ccccc34)cc2)cc1 ; c1ccc(-c2ccc(N(c3ccc(-n4c5ccccc5c5cc6ccccc6cc54)cc3)c3cc(-c4ccccc4)cc4ccccc34)cc2)cc1] 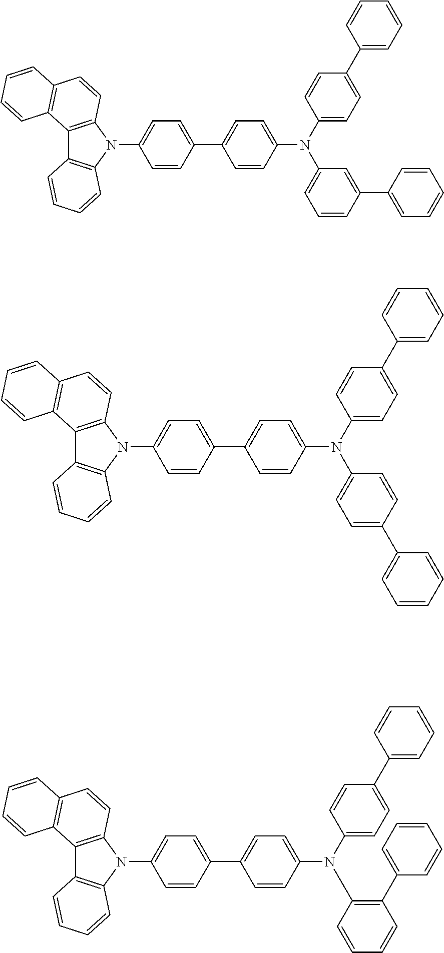 [SMILES: c1ccc(-c2ccc(N(c3ccc(-c4ccc(-n5c6ccccc6c6c7ccccc7ccc65)cc4)cc3)c3cccc(-c4ccccc4)c3)cc2)cc1 ; c1ccc(-c2ccc(N(c3ccc(-c4ccccc4)cc3)c3ccc(-c4ccc(-n5c6ccccc6c6c7ccccc7ccc65)cc4)cc3)cc2)cc1 ; c1ccc(-c2ccc(N(c3ccc(-c4ccc(-n5c6ccccc6c6c7ccccc7ccc65)cc4)cc3)c3ccccc3-c3ccccc3)cc2)cc1] 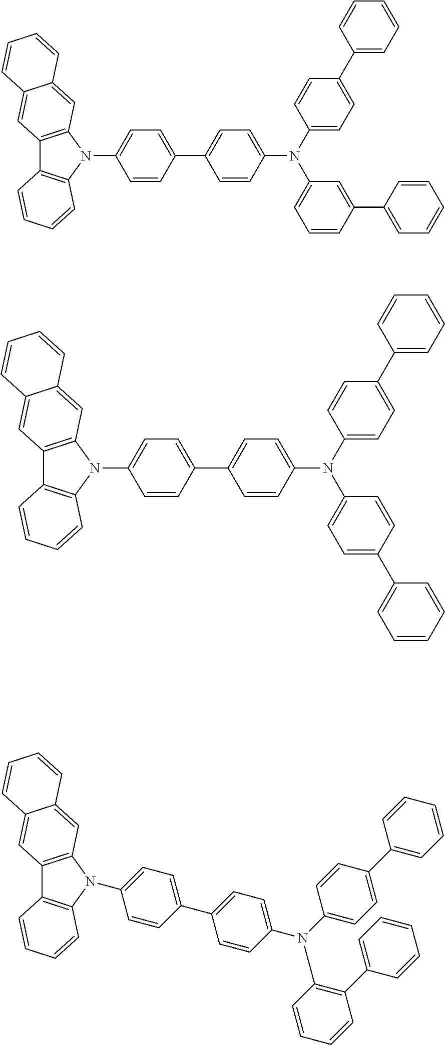 [SMILES: c1ccc(-c2ccc(N(c3ccc(-c4ccc(-n5c6ccccc6c6cc7ccccc7cc65)cc4)cc3)c3cccc(-c4ccccc4)c3)cc2)cc1 ; c1ccc(-c2ccc(N(c3ccc(-c4ccccc4)cc3)c3ccc(-c4ccc(-n5c6ccccc6c6cc7ccccc7cc65)cc4)cc3)cc2)cc1 ; c1ccc(-c2ccc(N(c3ccc(-c4ccc(-n5c6ccccc6c6cc7ccccc7cc65)cc4)cc3)c3ccccc3-c3ccccc3)cc2)cc1] 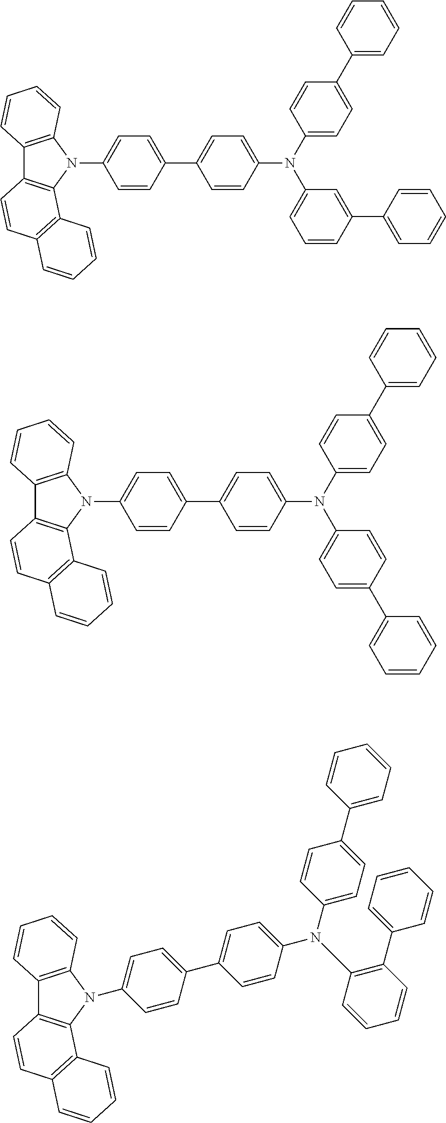 [SMILES: c1ccc(-c2ccc(N(c3ccc(-c4ccc(-n5c6ccccc6c6ccc7ccccc7c65)cc4)cc3)c3cccc(-c4ccccc4)c3)cc2)cc1 ; c1ccc(-c2ccc(N(c3ccc(-c4ccccc4)cc3)c3ccc(-c4ccc(-n5c6ccccc6c6ccc7ccccc7c65)cc4)cc3)cc2)cc1 ; c1ccc(-c2ccc(N(c3ccc(-c4ccc(-n5c6ccccc6c6ccc7ccccc7c65)cc4)cc3)c3ccccc3-c3ccccc3)cc2)cc1] 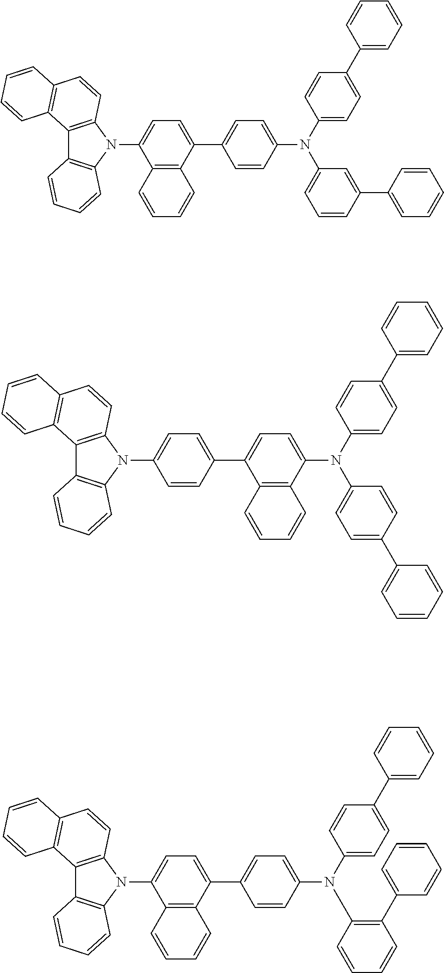 [SMILES: c1ccc(-c2ccc(N(c3ccc(-c4ccc(-n5c6ccccc6c6c7ccccc7ccc65)c5ccccc45)cc3)c3cccc(-c4ccccc4)c3)cc2)cc1 ; c1ccc(-c2ccc(N(c3ccc(-c4ccccc4)cc3)c3ccc(-c4ccc(-n5c6ccccc6c6c7ccccc7ccc65)cc4)c4ccccc34)cc2)cc1 ; c1ccc(-c2ccc(N(c3ccc(-c4ccc(-n5c6ccccc6c6c7ccccc7ccc65)c5ccccc45)cc3)c3ccccc3-c3ccccc3)cc2)cc1] 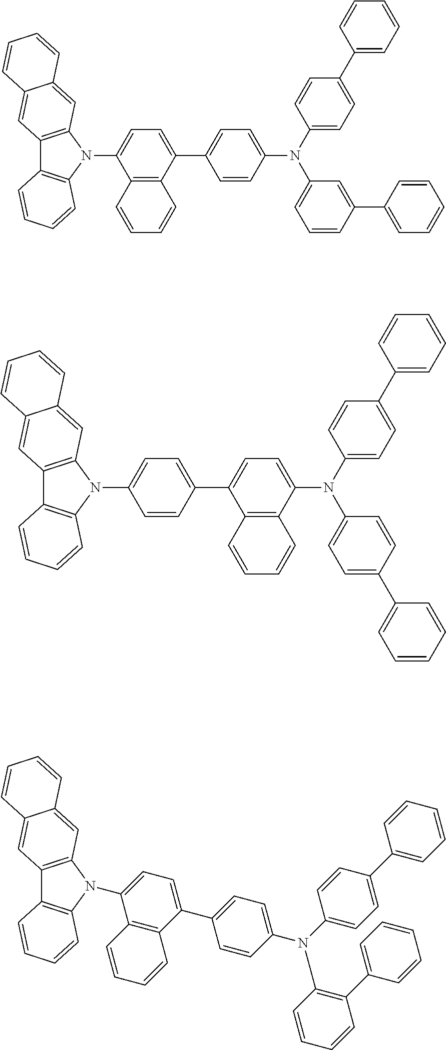 [SMILES: c1ccc(-c2ccc(N(c3ccc(-c4ccc(-n5c6ccccc6c6cc7ccccc7cc65)c5ccccc45)cc3)c3cccc(-c4ccccc4)c3)cc2)cc1 ; c1ccc(-c2ccc(N(c3ccc(-c4ccccc4)cc3)c3ccc(-c4ccc(-n5c6ccccc6c6cc7ccccc7cc65)cc4)c4ccccc34)cc2)cc1 ; c1ccc(-c2ccc(N(c3ccc(-c4ccc(-n5c6ccccc6c6cc7ccccc7cc65)c5ccccc45)cc3)c3ccccc3-c3ccccc3)cc2)cc1] 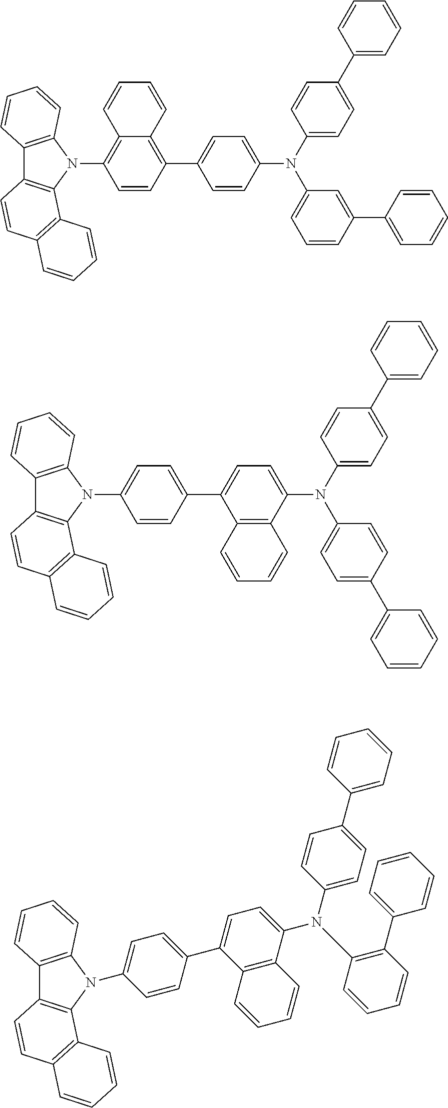 [SMILES: c1ccc(-c2ccc(N(c3ccc(-c4ccc(-n5c6ccccc6c6ccc7ccccc7c65)c5ccccc45)cc3)c3cccc(-c4ccccc4)c3)cc2)cc1 ; c1ccc(-c2ccc(N(c3ccc(-c4ccccc4)cc3)c3ccc(-c4ccc(-n5c6ccccc6c6ccc7ccccc7c65)cc4)c4ccccc34)cc2)cc1 ; c1ccc(-c2ccc(N(c3ccccc3-c3ccccc3)c3ccc(-c4ccc(-n5c6ccccc6c6ccc7ccccc7c65)cc4)c4ccccc34)cc2)cc1] 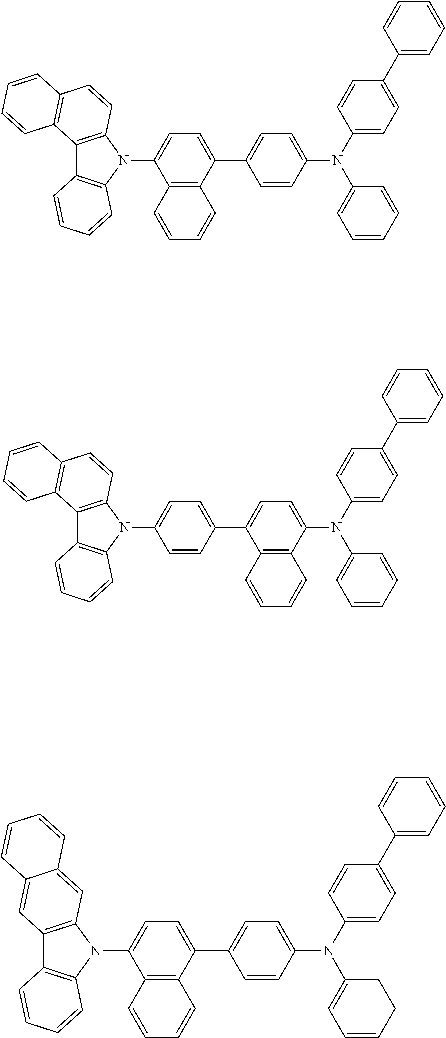 [SMILES: c1ccc(-c2ccc(N(c3ccccc3)c3ccc(-c4ccc(-n5c6ccccc6c6c7ccccc7ccc65)c5ccccc45)cc3)cc2)cc1 ; c1ccc(-c2ccc(N(c3ccccc3)c3ccc(-c4ccc(-n5c6ccccc6c6c7ccccc7ccc65)cc4)c4ccccc34)cc2)cc1 ; C1=CCCC(N(c2ccc(-c3ccccc3)cc2)c2ccc(-c3ccc(-n4c5ccccc5c5cc6ccccc6cc54)c4ccccc34)cc2)=C1] 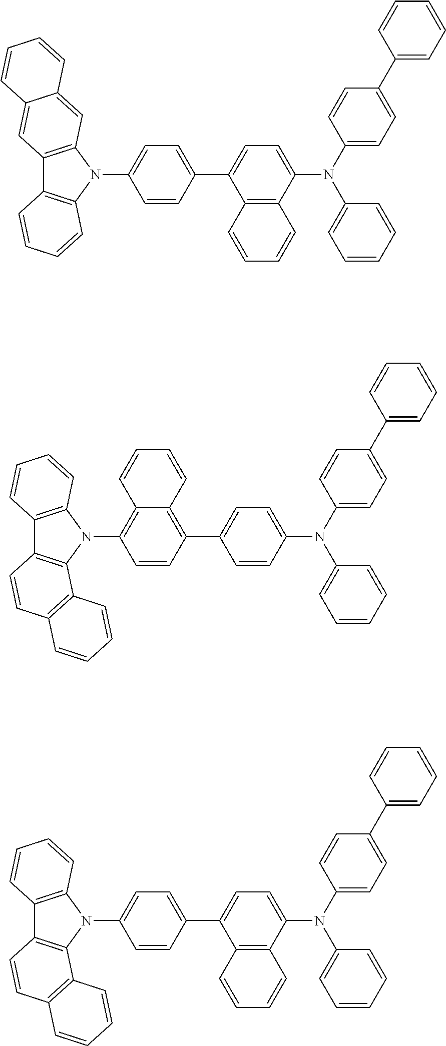 [SMILES: c1ccc(-c2ccc(N(c3ccccc3)c3ccc(-c4ccc(-n5c6ccccc6c6cc7ccccc7cc65)cc4)c4ccccc34)cc2)cc1 ; c1ccc(-c2ccc(N(c3ccccc3)c3ccc(-c4ccc(-n5c6ccccc6c6ccc7ccccc7c65)c5ccccc45)cc3)cc2)cc1 ; c1ccc(-c2ccc(N(c3ccccc3)c3ccc(-c4ccc(-n5c6ccccc6c6ccc7ccccc7c65)cc4)c4ccccc34)cc2)cc1] 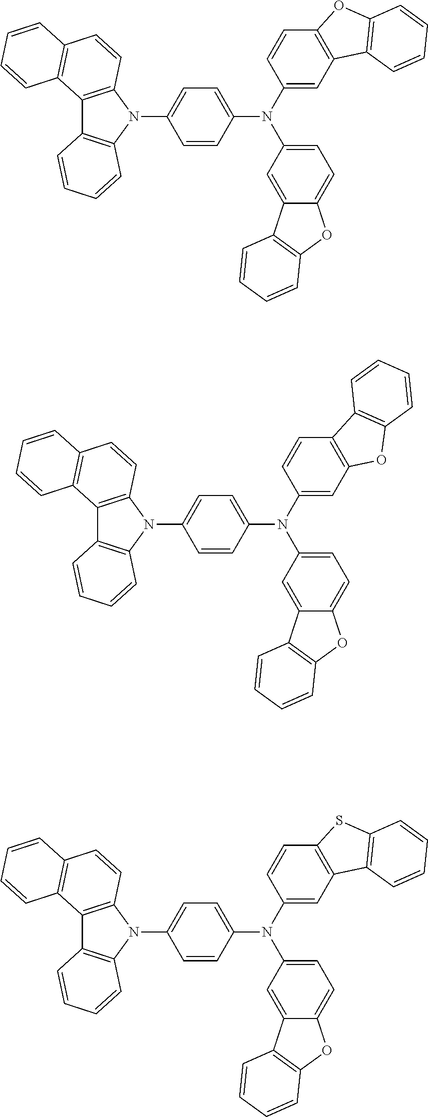 [SMILES: c1ccc2c(c1)ccc1c2c2ccccc2n1-c1ccc(N(c2ccc3oc4ccccc4c3c2)c2ccc3oc4ccccc4c3c2)cc1 ; c1ccc2c(c1)ccc1c2c2ccccc2n1-c1ccc(N(c2ccc3c(c2)oc2ccccc23)c2ccc3oc4ccccc4c3c2)cc1 ; c1ccc2c(c1)ccc1c2c2ccccc2n1-c1ccc(N(c2ccc3oc4ccccc4c3c2)c2ccc3sc4ccccc4c3c2)cc1] 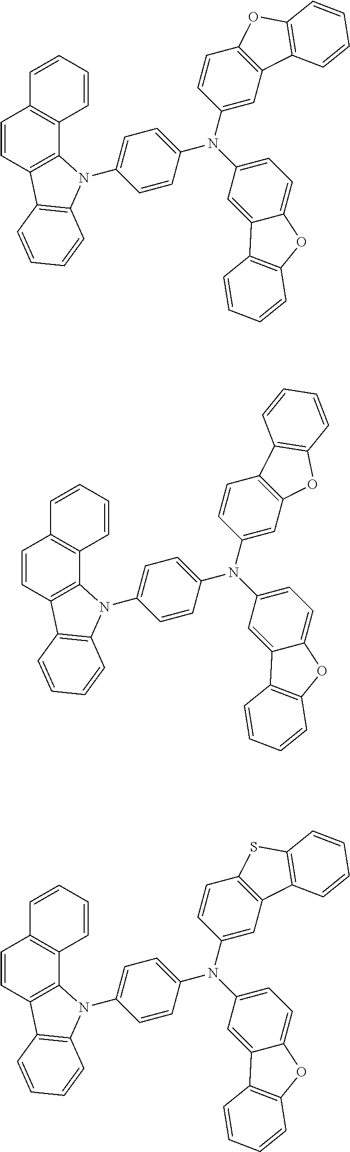 [SMILES: c1ccc2c(c1)ccc1c3ccccc3n(-c3ccc(N(c4ccc5oc6ccccc6c5c4)c4ccc5oc6ccccc6c5c4)cc3)c21 ; c1ccc2c(c1)ccc1c3ccccc3n(-c3ccc(N(c4ccc5c(c4)oc4ccccc45)c4ccc5oc6ccccc6c5c4)cc3)c21 ; c1ccc2c(c1)ccc1c3ccccc3n(-c3ccc(N(c4ccc5oc6ccccc6c5c4)c4ccc5sc6ccccc6c5c4)cc3)c21] 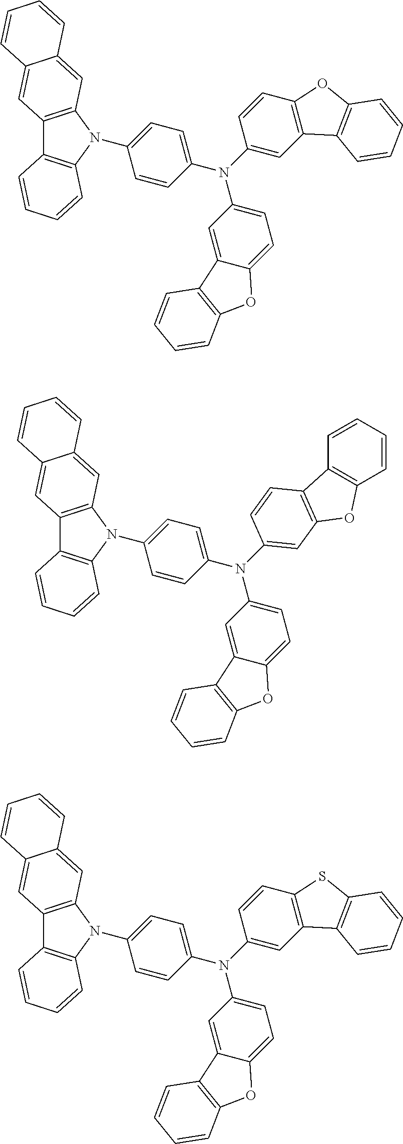 [SMILES: c1ccc2cc3c(cc2c1)c1ccccc1n3-c1ccc(N(c2ccc3oc4ccccc4c3c2)c2ccc3oc4ccccc4c3c2)cc1 ; c1ccc2cc3c(cc2c1)c1ccccc1n3-c1ccc(N(c2ccc3c(c2)oc2ccccc23)c2ccc3oc4ccccc4c3c2)cc1 ; c1ccc2cc3c(cc2c1)c1ccccc1n3-c1ccc(N(c2ccc3oc4ccccc4c3c2)c2ccc3sc4ccccc4c3c2)cc1] 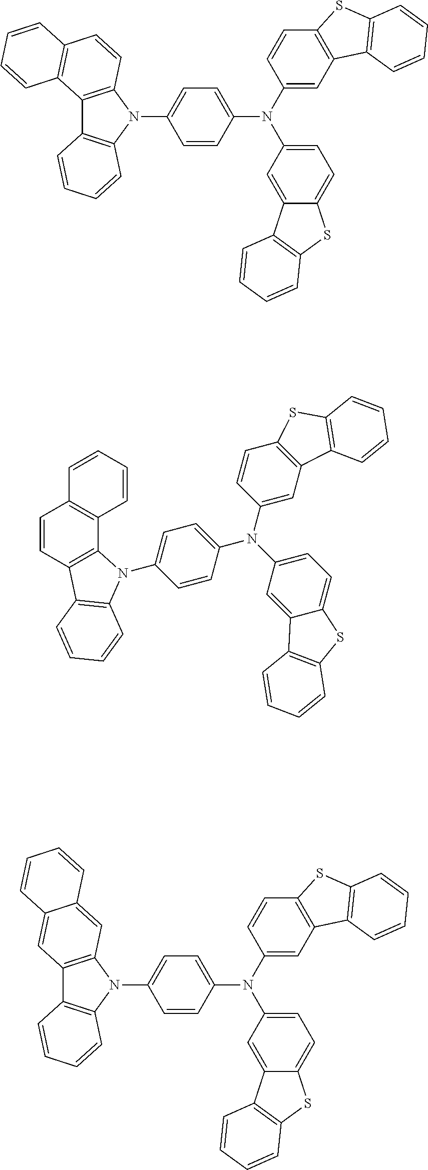 [SMILES: c1ccc2c(c1)ccc1c2c2ccccc2n1-c1ccc(N(c2ccc3sc4ccccc4c3c2)c2ccc3sc4ccccc4c3c2)cc1 ; c1ccc2c(c1)ccc1c3ccccc3n(-c3ccc(N(c4ccc5sc6ccccc6c5c4)c4ccc5sc6ccccc6c5c4)cc3)c21 ; c1ccc2cc3c(cc2c1)c1ccccc1n3-c1ccc(N(c2ccc3sc4ccccc4c3c2)c2ccc3sc4ccccc4c3c2)cc1] 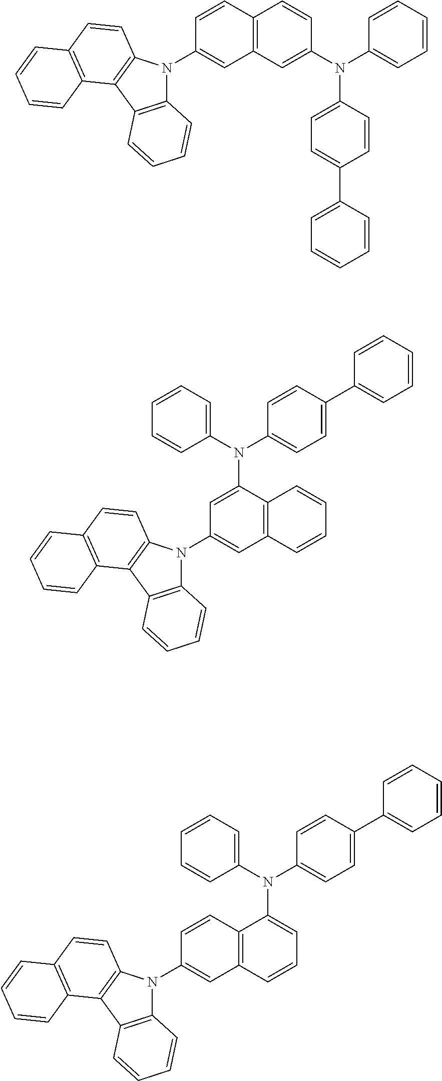 [SMILES: c1ccc(-c2ccc(N(c3ccccc3)c3ccc4ccc(-n5c6ccccc6c6c7ccccc7ccc65)cc4c3)cc2)cc1 ; c1ccc(-c2ccc(N(c3ccccc3)c3cc(-n4c5ccccc5c5c6ccccc6ccc54)cc4ccccc34)cc2)cc1 ; c1ccc(-c2ccc(N(c3ccccc3)c3cccc4cc(-n5c6ccccc6c6c7ccccc7ccc65)ccc34)cc2)cc1] 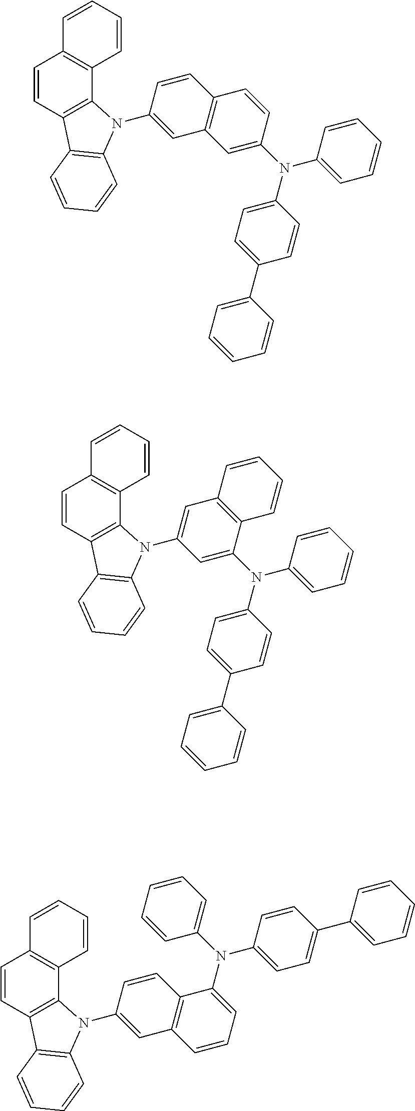 [SMILES: c1ccc(-c2ccc(N(c3ccccc3)c3ccc4ccc(-n5c6ccccc6c6ccc7ccccc7c65)cc4c3)cc2)cc1 ; c1ccc(-c2ccc(N(c3ccccc3)c3cc(-n4c5ccccc5c5ccc6ccccc6c54)cc4ccccc34)cc2)cc1 ; c1ccc(-c2ccc(N(c3ccccc3)c3cccc4cc(-n5c6ccccc6c6ccc7ccccc7c65)ccc34)cc2)cc1] 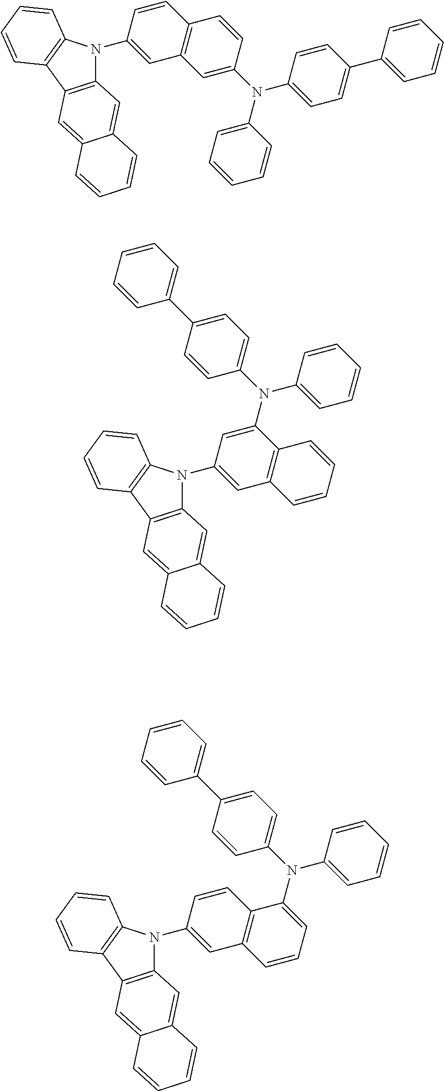 [SMILES: c1ccc(-c2ccc(N(c3ccccc3)c3ccc4ccc(-n5c6ccccc6c6cc7ccccc7cc65)cc4c3)cc2)cc1 ; c1ccc(-c2ccc(N(c3ccccc3)c3cc(-n4c5ccccc5c5cc6ccccc6cc54)cc4ccccc34)cc2)cc1 ; c1ccc(-c2ccc(N(c3ccccc3)c3cccc4cc(-n5c6ccccc6c6cc7ccccc7cc65)ccc34)cc2)cc1] 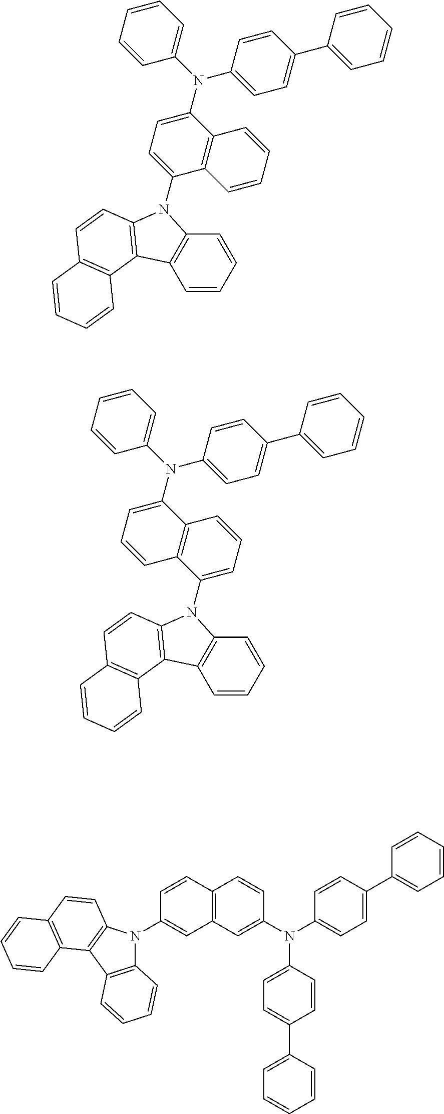 [SMILES: c1ccc(-c2ccc(N(c3ccccc3)c3ccc(-n4c5ccccc5c5c6ccccc6ccc54)c4ccccc34)cc2)cc1 ; c1ccc(-c2ccc(N(c3ccccc3)c3cccc4c(-n5c6ccccc6c6c7ccccc7ccc65)cccc34)cc2)cc1 ; c1ccc(-c2ccc(N(c3ccc(-c4ccccc4)cc3)c3ccc4ccc(-n5c6ccccc6c6c7ccccc7ccc65)cc4c3)cc2)cc1] 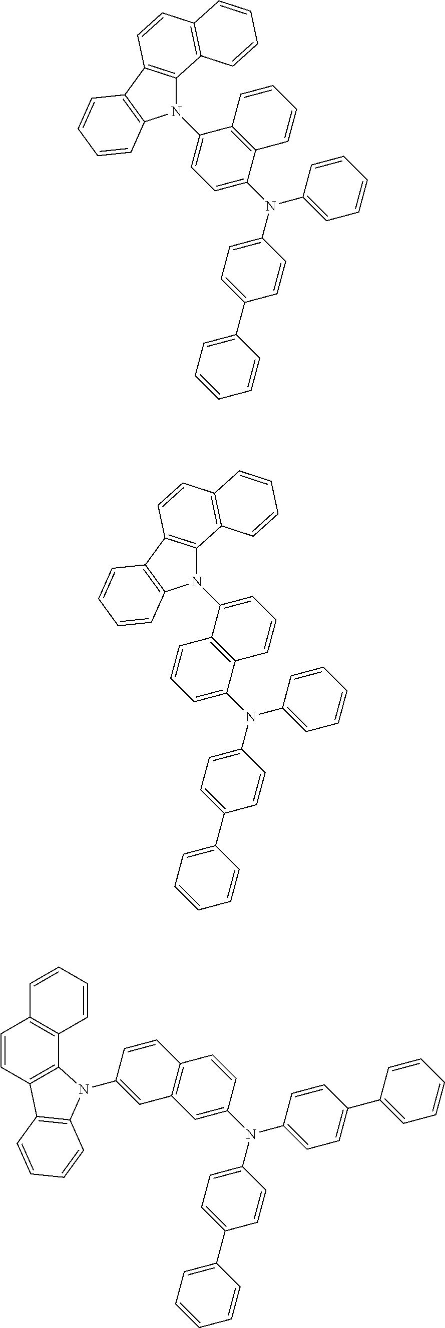 [SMILES: c1ccc(-c2ccc(N(c3ccccc3)c3ccc(-n4c5ccccc5c5ccc6ccccc6c54)c4ccccc34)cc2)cc1 ; c1ccc(-c2ccc(N(c3ccccc3)c3cccc4c(-n5c6ccccc6c6ccc7ccccc7c65)cccc34)cc2)cc1 ; c1ccc(-c2ccc(N(c3ccc(-c4ccccc4)cc3)c3ccc4ccc(-n5c6ccccc6c6ccc7ccccc7c65)cc4c3)cc2)cc1] 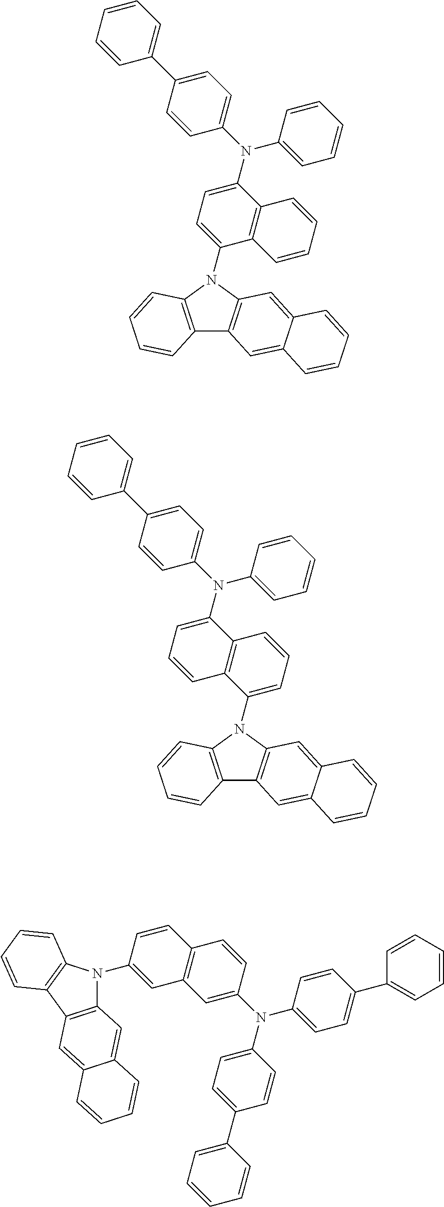 [SMILES: c1ccc(-c2ccc(N(c3ccccc3)c3ccc(-n4c5ccccc5c5cc6ccccc6cc54)c4ccccc34)cc2)cc1 ; c1ccc(-c2ccc(N(c3ccccc3)c3cccc4c(-n5c6ccccc6c6cc7ccccc7cc65)cccc34)cc2)cc1 ; c1ccc(-c2ccc(N(c3ccc(-c4ccccc4)cc3)c3ccc4ccc(-n5c6ccccc6c6cc7ccccc7cc65)cc4c3)cc2)cc1] 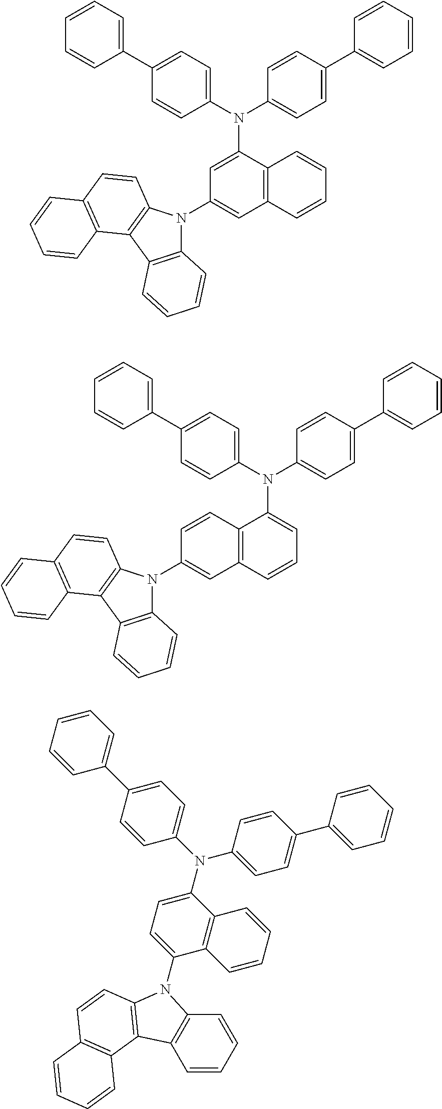 [SMILES: c1ccc(-c2ccc(N(c3ccc(-c4ccccc4)cc3)c3cc(-n4c5ccccc5c5c6ccccc6ccc54)cc4ccccc34)cc2)cc1 ; c1ccc(-c2ccc(N(c3ccc(-c4ccccc4)cc3)c3cccc4cc(-n5c6ccccc6c6c7ccccc7ccc65)ccc34)cc2)cc1 ; c1ccc(-c2ccc(N(c3ccc(-c4ccccc4)cc3)c3ccc(-n4c5ccccc5c5c6ccccc6ccc54)c4ccccc34)cc2)cc1] 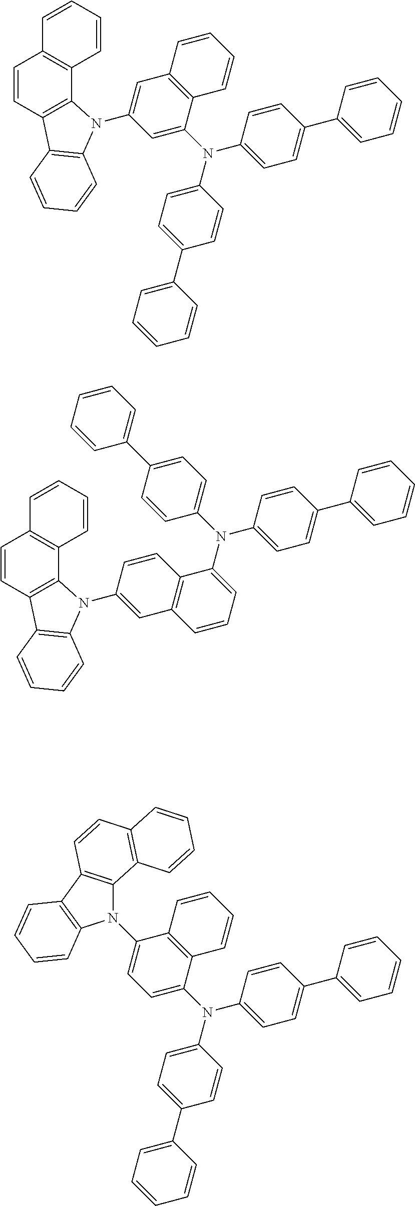 [SMILES: c1ccc(-c2ccc(N(c3ccc(-c4ccccc4)cc3)c3cc(-n4c5ccccc5c5ccc6ccccc6c54)cc4ccccc34)cc2)cc1 ; c1ccc(-c2ccc(N(c3ccc(-c4ccccc4)cc3)c3cccc4cc(-n5c6ccccc6c6ccc7ccccc7c65)ccc34)cc2)cc1 ; c1ccc(-c2ccc(N(c3ccc(-c4ccccc4)cc3)c3ccc(-n4c5ccccc5c5ccc6ccccc6c54)c4ccccc34)cc2)cc1] 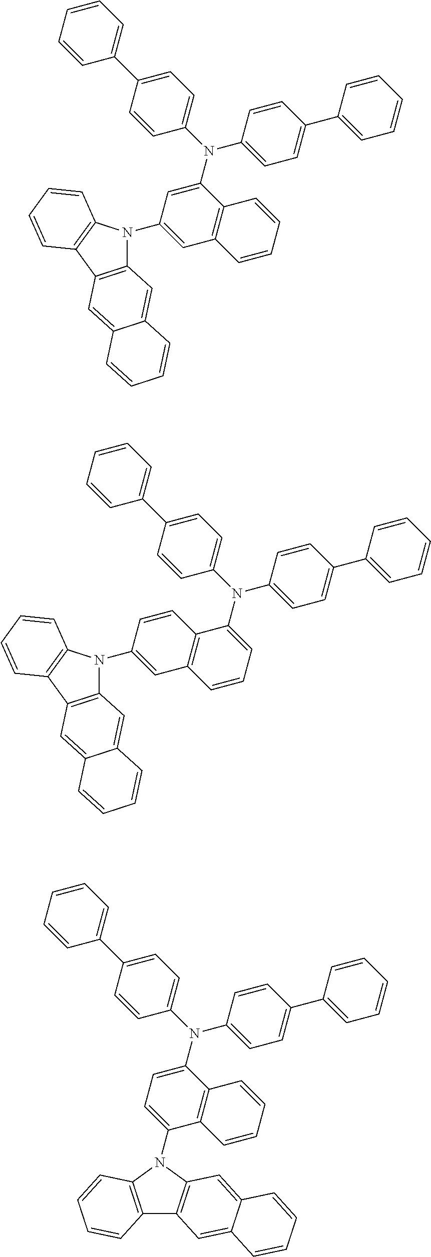 [SMILES: c1ccc(-c2ccc(N(c3ccc(-c4ccccc4)cc3)c3cc(-n4c5ccccc5c5cc6ccccc6cc54)cc4ccccc34)cc2)cc1 ; c1ccc(-c2ccc(N(c3ccc(-c4ccccc4)cc3)c3cccc4cc(-n5c6ccccc6c6cc7ccccc7cc65)ccc34)cc2)cc1 ; c1ccc(-c2ccc(N(c3ccc(-c4ccccc4)cc3)c3ccc(-n4c5ccccc5c5cc6ccccc6cc54)c4ccccc34)cc2)cc1] 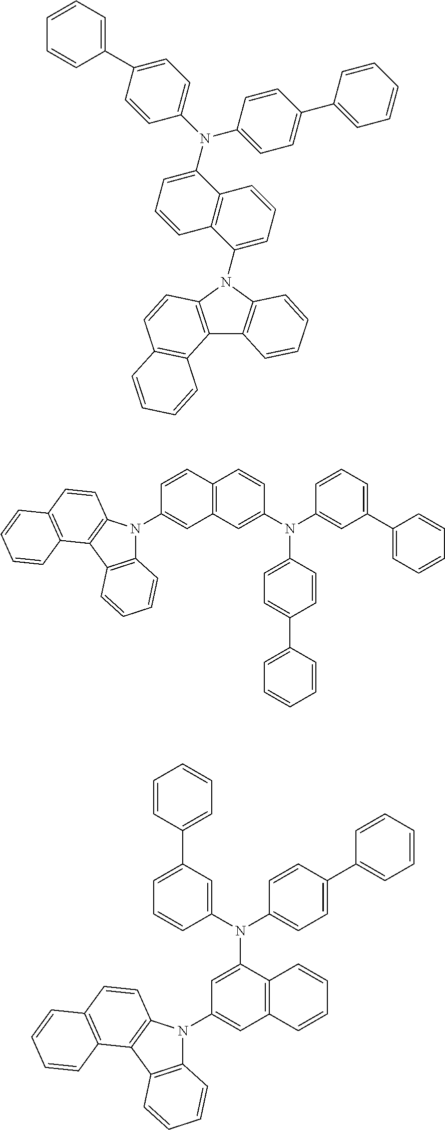 [SMILES: c1ccc(-c2ccc(N(c3ccc(-c4ccccc4)cc3)c3cccc4c(-n5c6ccccc6c6c7ccccc7ccc65)cccc34)cc2)cc1 ; c1ccc(-c2ccc(N(c3cccc(-c4ccccc4)c3)c3ccc4ccc(-n5c6ccccc6c6c7ccccc7ccc65)cc4c3)cc2)cc1 ; c1ccc(-c2ccc(N(c3cccc(-c4ccccc4)c3)c3cc(-n4c5ccccc5c5c6ccccc6ccc54)cc4ccccc34)cc2)cc1] 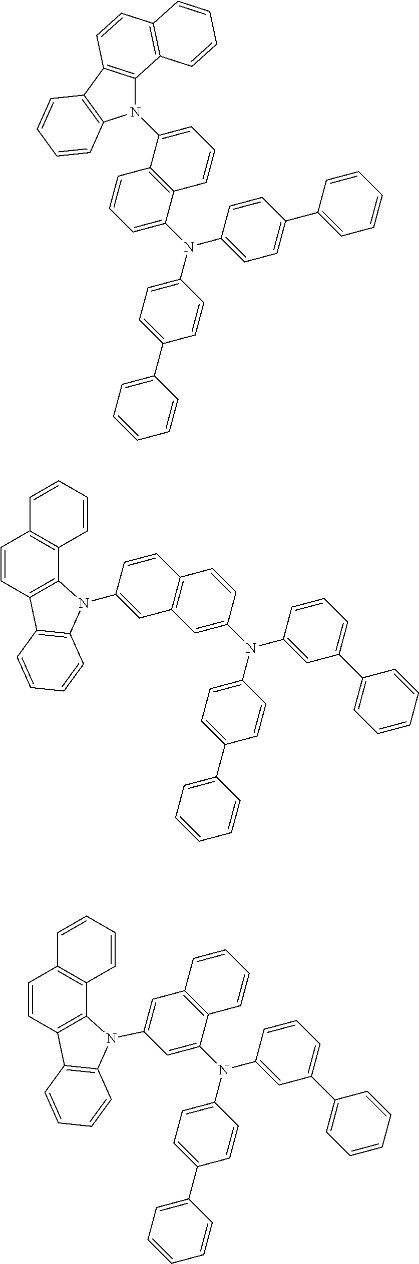 [SMILES: c1ccc(-c2ccc(N(c3ccc(-c4ccccc4)cc3)c3cccc4c(-n5c6ccccc6c6ccc7ccccc7c65)cccc34)cc2)cc1 ; c1ccc(-c2ccc(N(c3cccc(-c4ccccc4)c3)c3ccc4ccc(-n5c6ccccc6c6ccc7ccccc7c65)cc4c3)cc2)cc1 ; c1ccc(-c2ccc(N(c3cccc(-c4ccccc4)c3)c3cc(-n4c5ccccc5c5ccc6ccccc6c54)cc4ccccc34)cc2)cc1] 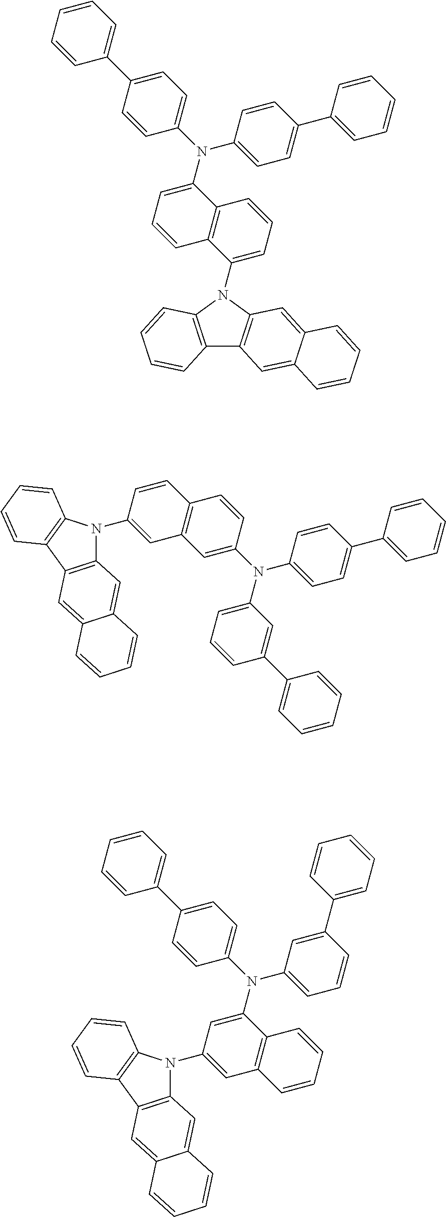 [SMILES: c1ccc(-c2ccc(N(c3ccc(-c4ccccc4)cc3)c3cccc4c(-n5c6ccccc6c6cc7ccccc7cc65)cccc34)cc2)cc1 ; c1ccc(-c2ccc(N(c3cccc(-c4ccccc4)c3)c3ccc4ccc(-n5c6ccccc6c6cc7ccccc7cc65)cc4c3)cc2)cc1 ; c1ccc(-c2ccc(N(c3cccc(-c4ccccc4)c3)c3cc(-n4c5ccccc5c5cc6ccccc6cc54)cc4ccccc34)cc2)cc1] 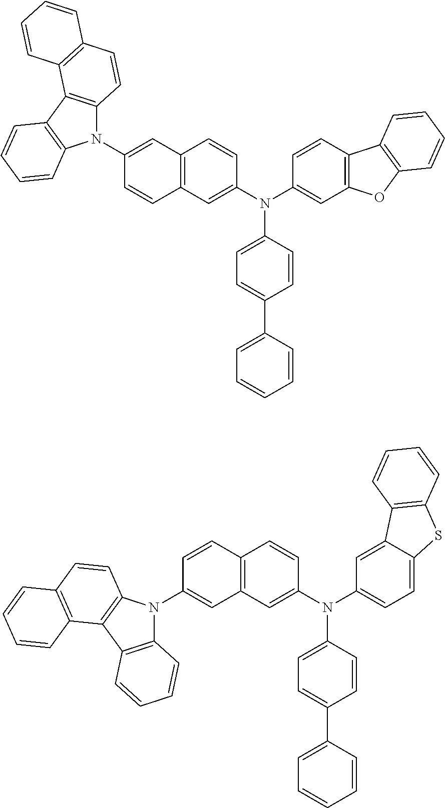 [SMILES: c1ccc(-c2ccc(N(c3ccc4cc(-n5c6ccccc6c6c7ccccc7ccc65)ccc4c3)c3ccc4c(c3)oc3ccccc34)cc2)cc1 ; c1ccc(-c2ccc(N(c3ccc4ccc(-n5c6ccccc6c6c7ccccc7ccc65)cc4c3)c3ccc4sc5ccccc5c4c3)cc2)cc1] 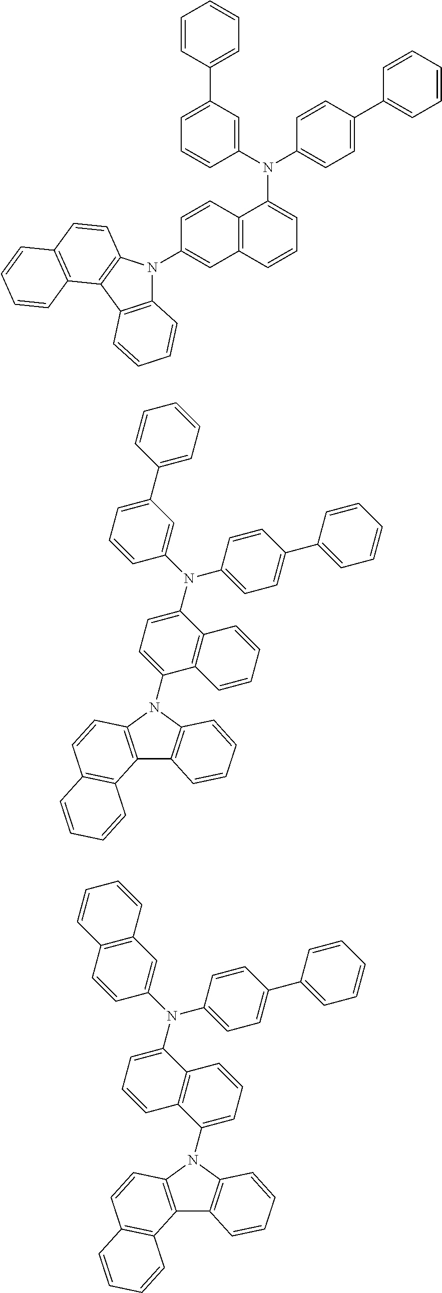 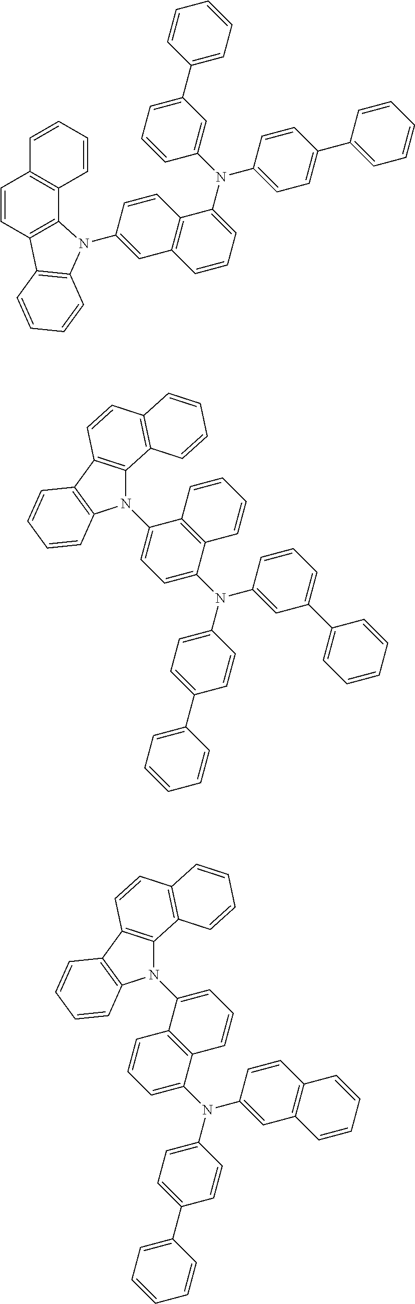 [SMILES: c1ccc(-c2ccc(N(c3cccc(-c4ccccc4)c3)c3cccc4cc(-n5c6ccccc6c6ccc7ccccc7c65)ccc34)cc2)cc1 ; c1ccc(-c2ccc(N(c3cccc(-c4ccccc4)c3)c3ccc(-n4c5ccccc5c5ccc6ccccc6c54)c4ccccc34)cc2)cc1 ; c1ccc(-c2ccc(N(c3ccc4ccccc4c3)c3cccc4c(-n5c6ccccc6c6ccc7ccccc7c65)cccc34)cc2)cc1] 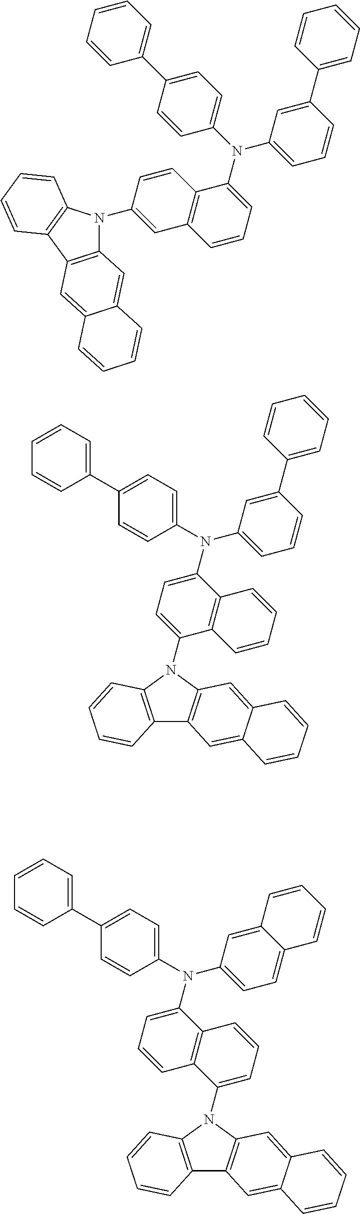 [SMILES: c1ccc(-c2ccc(N(c3cccc(-c4ccccc4)c3)c3cccc4cc(-n5c6ccccc6c6cc7ccccc7cc65)ccc34)cc2)cc1 ; c1ccc(-c2ccc(N(c3cccc(-c4ccccc4)c3)c3ccc(-n4c5ccccc5c5cc6ccccc6cc54)c4ccccc34)cc2)cc1 ; c1ccc(-c2ccc(N(c3ccc4ccccc4c3)c3cccc4c(-n5c6ccccc6c6cc7ccccc7cc65)cccc34)cc2)cc1] 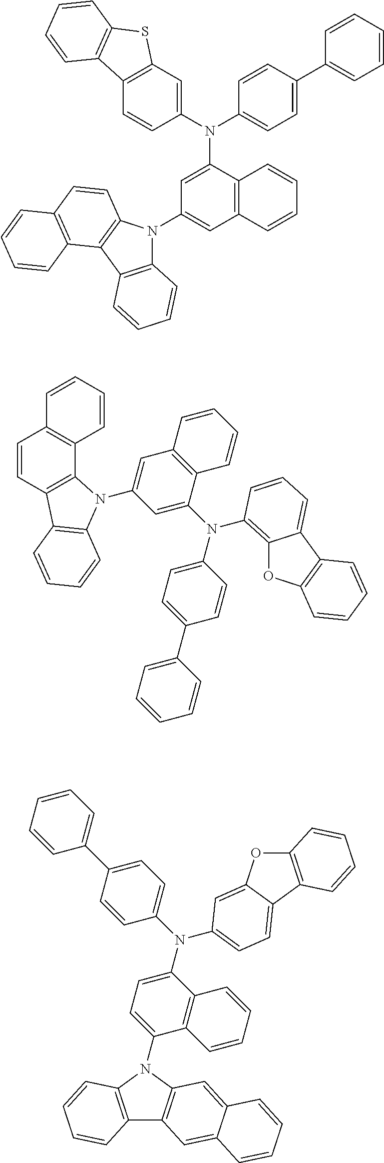 [SMILES: c1ccc(-c2ccc(N(c3ccc4c(c3)sc3ccccc34)c3cc(-n4c5ccccc5c5c6ccccc6ccc54)cc4ccccc34)cc2)cc1 ; c1ccc(-c2ccc(N(c3cc(-n4c5ccccc5c5ccc6ccccc6c54)cc4ccccc34)c3cccc4c3oc3ccccc34)cc2)cc1 ; c1ccc(-c2ccc(N(c3ccc4c(c3)oc3ccccc34)c3ccc(-n4c5ccccc5c5cc6ccccc6cc54)c4ccccc34)cc2)cc1] 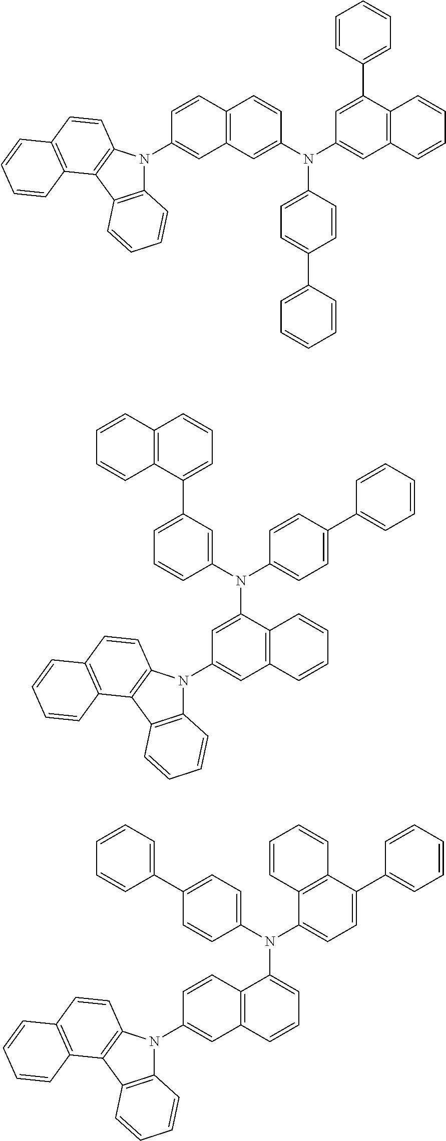 [SMILES: c1ccc(-c2ccc(N(c3ccc4ccc(-n5c6ccccc6c6c7ccccc7ccc65)cc4c3)c3cc(-c4ccccc4)c4ccccc4c3)cc2)cc1 ; c1ccc(-c2ccc(N(c3cccc(-c4cccc5ccccc45)c3)c3cc(-n4c5ccccc5c5c6ccccc6ccc54)cc4ccccc34)cc2)cc1 ; c1ccc(-c2ccc(N(c3cccc4cc(-n5c6ccccc6c6c7ccccc7ccc65)ccc34)c3ccc(-c4ccccc4)c4ccccc34)cc2)cc1] 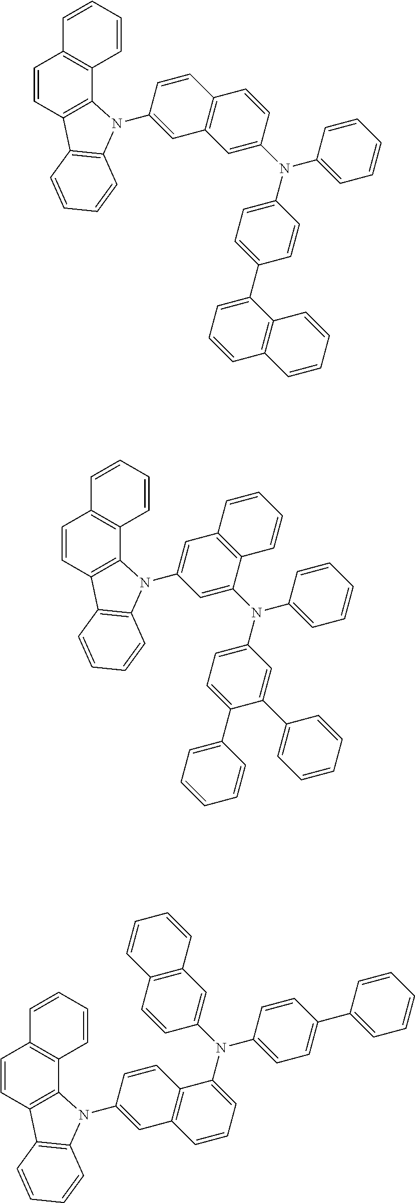 [SMILES: c1ccc(N(c2ccc(-c3cccc4ccccc34)cc2)c2ccc3ccc(-n4c5ccccc5c5ccc6ccccc6c54)cc3c2)cc1 ; c1ccc(-c2ccc(N(c3ccccc3)c3cc(-n4c5ccccc5c5ccc6ccccc6c54)cc4ccccc34)cc2-c2ccccc2)cc1 ; c1ccc(-c2ccc(N(c3ccc4ccccc4c3)c3cccc4cc(-n5c6ccccc6c6ccc7ccccc7c65)ccc34)cc2)cc1] 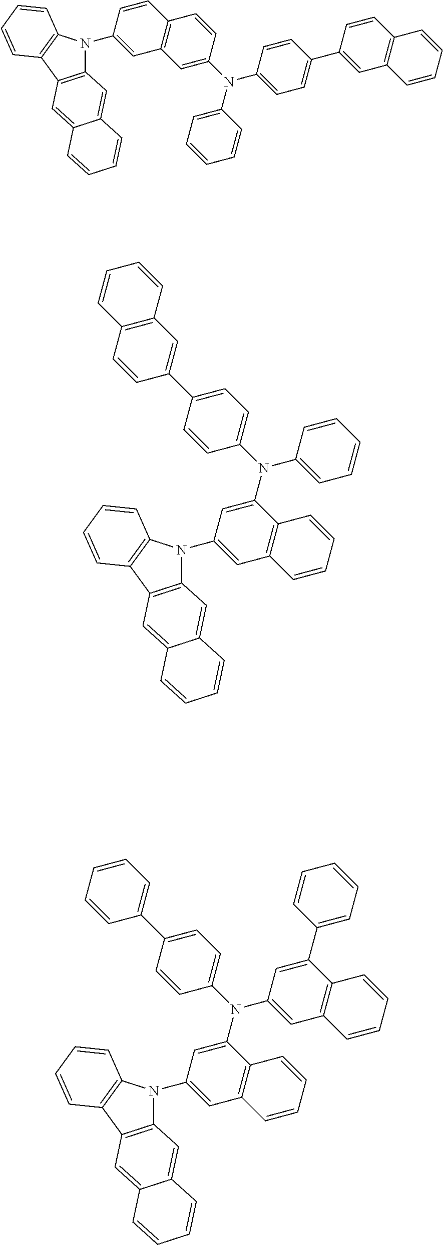 [SMILES: c1ccc(N(c2ccc(-c3ccc4ccccc4c3)cc2)c2ccc3ccc(-n4c5ccccc5c5cc6ccccc6cc54)cc3c2)cc1 ; c1ccc(N(c2ccc(-c3ccc4ccccc4c3)cc2)c2cc(-n3c4ccccc4c4cc5ccccc5cc43)cc3ccccc23)cc1 ; c1ccc(-c2ccc(N(c3cc(-c4ccccc4)c4ccccc4c3)c3cc(-n4c5ccccc5c5cc6ccccc6cc54)cc4ccccc34)cc2)cc1] 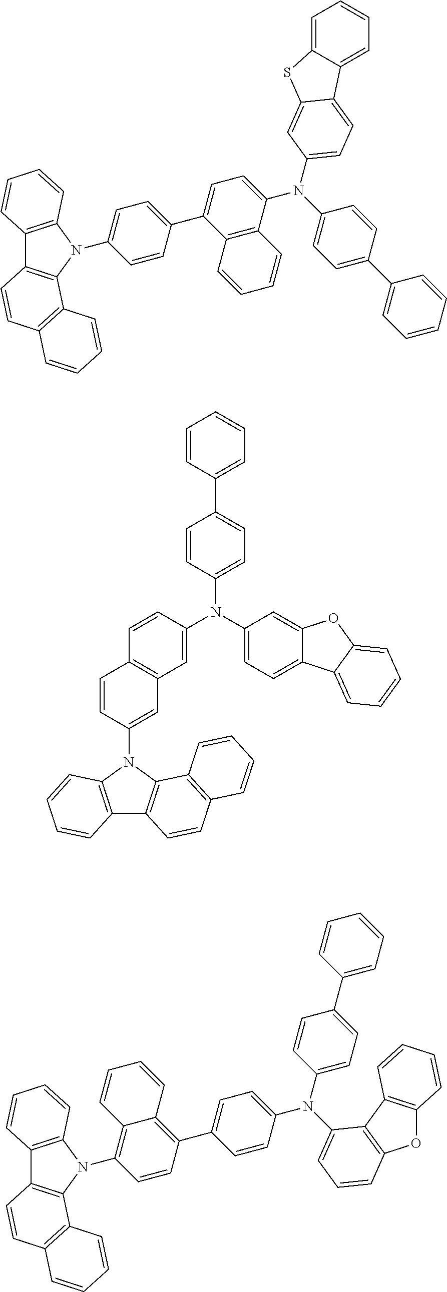 [SMILES: c1ccc(-c2ccc(N(c3ccc4c(c3)sc3ccccc34)c3ccc(-c4ccc(-n5c6ccccc6c6ccc7ccccc7c65)cc4)c4ccccc34)cc2)cc1 ; c1ccc(-c2ccc(N(c3ccc4ccc(-n5c6ccccc6c6ccc7ccccc7c65)cc4c3)c3ccc4c(c3)oc3ccccc34)cc2)cc1 ; c1ccc(-c2ccc(N(c3ccc(-c4ccc(-n5c6ccccc6c6ccc7ccccc7c65)c5ccccc45)cc3)c3cccc4oc5ccccc5c34)cc2)cc1] 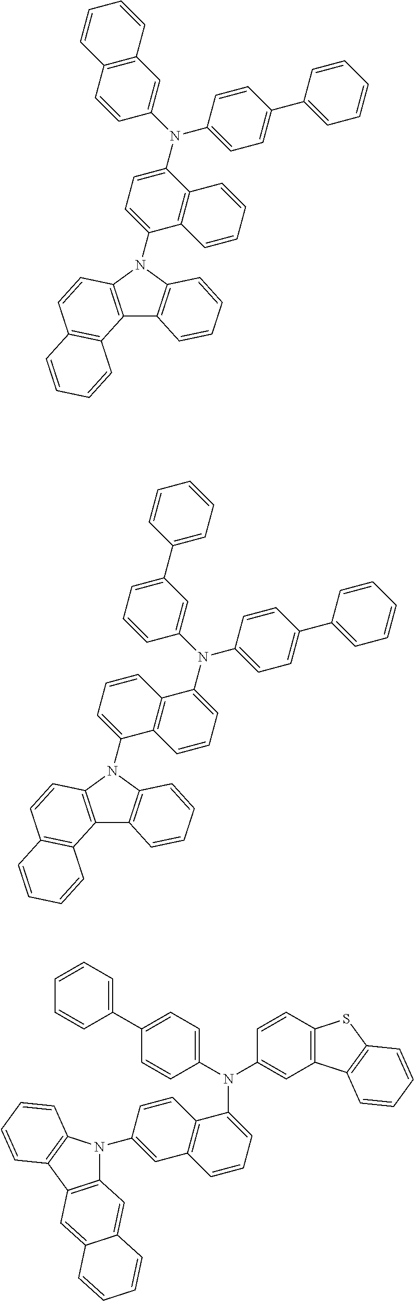 [SMILES: c1ccc(-c2ccc(N(c3ccc4ccccc4c3)c3ccc(-n4c5ccccc5c5c6ccccc6ccc54)c4ccccc34)cc2)cc1 ; c1ccc(-c2ccc(N(c3cccc(-c4ccccc4)c3)c3cccc4c(-n5c6ccccc6c6c7ccccc7ccc65)cccc34)cc2)cc1 ; c1ccc(-c2ccc(N(c3ccc4sc5ccccc5c4c3)c3cccc4cc(-n5c6ccccc6c6cc7ccccc7cc65)ccc34)cc2)cc1] 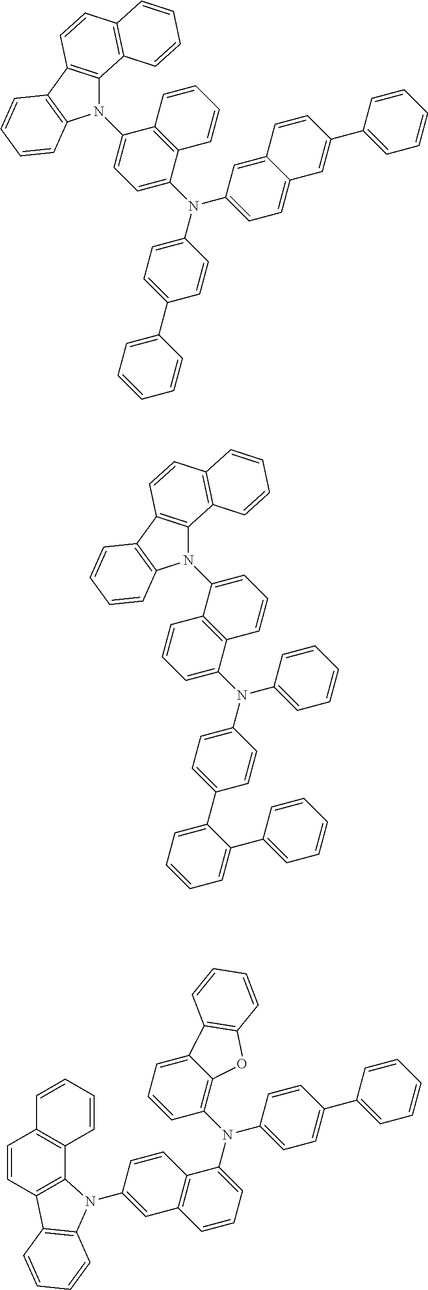 [SMILES: c1ccc(-c2ccc(N(c3ccc4cc(-c5ccccc5)ccc4c3)c3ccc(-n4c5ccccc5c5ccc6ccccc6c54)c4ccccc34)cc2)cc1 ; c1ccc(-c2ccccc2-c2ccc(N(c3ccccc3)c3cccc4c(-n5c6ccccc6c6ccc7ccccc7c65)cccc34)cc2)cc1 ; c1ccc(-c2ccc(N(c3cccc4cc(-n5c6ccccc6c6ccc7ccccc7c65)ccc34)c3cccc4c3oc3ccccc34)cc2)cc1] 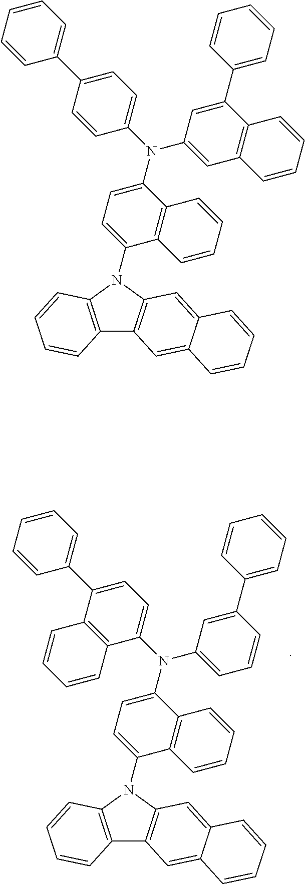 [SMILES: c1ccc(-c2ccc(N(c3cc(-c4ccccc4)c4ccccc4c3)c3ccc(-n4c5ccccc5c5cc6ccccc6cc54)c4ccccc34)cc2)cc1 ; c1ccc(-c2cccc(N(c3ccc(-c4ccccc4)c4ccccc34)c3ccc(-n4c5ccccc5c5cc6ccccc6cc54)c4ccccc34)c2)cc1]
